(19) United States
(12) Patent Application Publication
(10) Pub. No.: US 2024/0251665 A1
(43) Pub. Date: Jul. 25, 2024

(54) TRANSVERSE CURRENT SUPPRESSING MATERIAL, CARBAZOLE COMPOUND, HOLE-INJECTION LAYER AND ORGANIC ELECTROLUMINESCENT ELEMENT

(71) Applicant: TOSOH CORPORATION, Yamaguchi (JP)

(72) Inventors: Masaya HIRANO, Yamaguchi (JP); Naoki MATSUMOTO, Yamaguchi (JP); Hirokazu SHINYA, Yamaguchi (JP); Shintaro NOMURA, Yamaguchi (JP); Hiroyuki KAWASHIMA, Yamaguchi (JP); Kenji KOIKE, Yamaguchi (JP)

(73) Assignee: TOSOH CORPORATION, Yamaguchi (JP)

(21) Appl. No.: 18/285,084

(22) PCT Filed: Mar. 31, 2022
(86) PCT No.: PCT/JP2022/016975

§ 371 Date: Sep. 29, 2023

(30) Foreign Application Priority Data
Mar. 31, 2021 (JP) 2021-061639

(51) Int. Cl. H10K 85/60; C07D 209/88; C07D 405/04; C07D 405/10; C07D 405/12; C07D 405/14; C07D 409/04; C07D 409/10; C07D 409/12; C07D 409/14; C09K 11/06; H10K 50/15; H10K 50/17; H10K 85/40

(52) U.S. Cl. CPC H10K 85/636; C07D 209/88; C07D 405/04; C07D 405/10; C07D 405/12; C07D 405/14; C07D 409/04; C07D 409/10; C07D 409/12; C07D 409/14; C09K 11/06; H10K 85/40; H10K 85/633; C09K 2211/1018; H10K 50/15; H10K 50/17; H10K 85/615; H10K 85/622; H10K 85/624; H10K 85/626; H10K 85/6572; H10K 85/6574; H10K 85/6576

(57) ABSTRACT

There is provided a transverse current suppressing material that suppresses a transverse current of an organic electroluminescent element, a carbazole compound, a hole-injection layer containing the transverse current suppressing material and the carbazole compound and an organic electroluminescent element that has good drive voltage, luminous efficiency and durability and has a lower transverse current. A transverse current suppressing material for an organic electroluminescent element, represented by the formula (1) [In the formula (1), A is represented by the formula (2) or (3), and B is represented by the formula (4)].

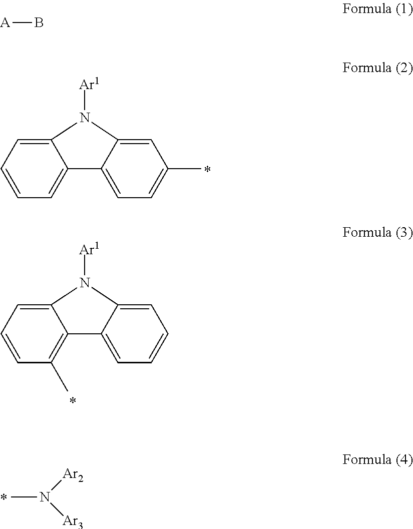

### TECHNICAL FIELD

The present disclosure relates to a transverse current suppressing material for an organic electroluminescent element, a carbazole compound, a hole-injection layer and an organic electroluminescent element.

### BACKGROUND ART

In a hole-injection layer of an organic electroluminescent element, an electron-donating triarylamine compound is doped with an electron-accepting p-dopant. Doping the triarylamine compound with a p-dopant can generate a hole, increase the number of holes injected into the organic electroluminescent element and reduce the drive voltage of the element. In general, when an electric field is applied to an organic electroluminescent element, a hole moves vertically from the positive electrode to the negative electrode in the direction of the electric field. In a hole-injection layer in which a triarylamine compound is doped with a p-dopant, a hole thus generated moves easily and freely and sometimes moves in a horizontal direction with respect to a positive electrode film. In general, in an organic electroluminescent display, a hole-injection layer and a hole-transport layer are commonly used for a plurality of pixels, and, when a transverse current is generated as described above, an unintended pixel emits light and impairs image quality. For example, Non-patent Document 1 discloses crosstalk as a phenomenon in which an adjacent pixel emits light.

### PRIOR ART DOCUMENTS

### Non-Patent Document

Non-patent Document 1: Journal of Information Display, 2018, vol. 19, p. 61

### DISCLOSURE OF INVENTION

### Technical Problem

An organic electroluminescent display in which a common hole-injection layer and a common hole-transport layer are applied to a plurality of pixels has the problem that a transverse current flowing in a horizontal direction with respect to a positive electrode film is generated in a known hole-injection layer and a known hole-transport layer and impairs the image quality of the organic electroluminescent display.

Accordingly, one aspect of the present disclosure is directed to providing a transverse current suppressing material that suppresses a transverse current of an organic electroluminescent element, a carbazole compound, a hole-injection layer containing the transverse current suppressing material and the carbazole compound and an organic electroluminescent element that has good drive voltage, luminous efficiency and durability and has a lower transverse current.

### Solution to Problem

According to one aspect of the present disclosure, there is provided a transverse current suppressing material for an electroluminescent element, represented by the formula (1):

[1] A transverse current suppressing material for an organic electroluminescent element, represented by the formula (1):

[Chem. 1]

A-B Formula (1)

wherein
A is represented by the formula (2) or (3); and
B is represented by the formula (4);

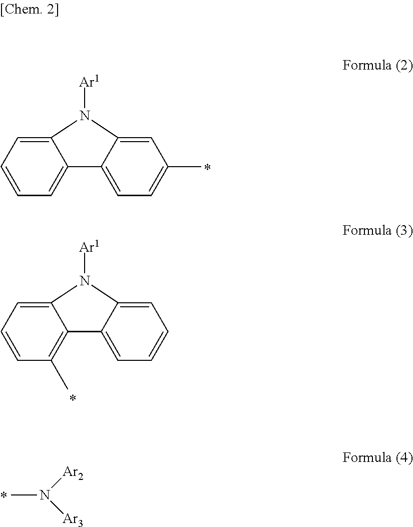

wherein
Ar¹ to Ar³ each independently denote
an optionally substituted monocyclic, linked or fused aromatic hydrocarbon group with 6 to 30 carbon atoms or
an optionally substituted monocyclic, linked or fused heteroaromatic group with 3 to 30 carbon atoms;
at least one of Ar¹ to Ar³ denotes a group represented by any one of the formulae (5) to (21);

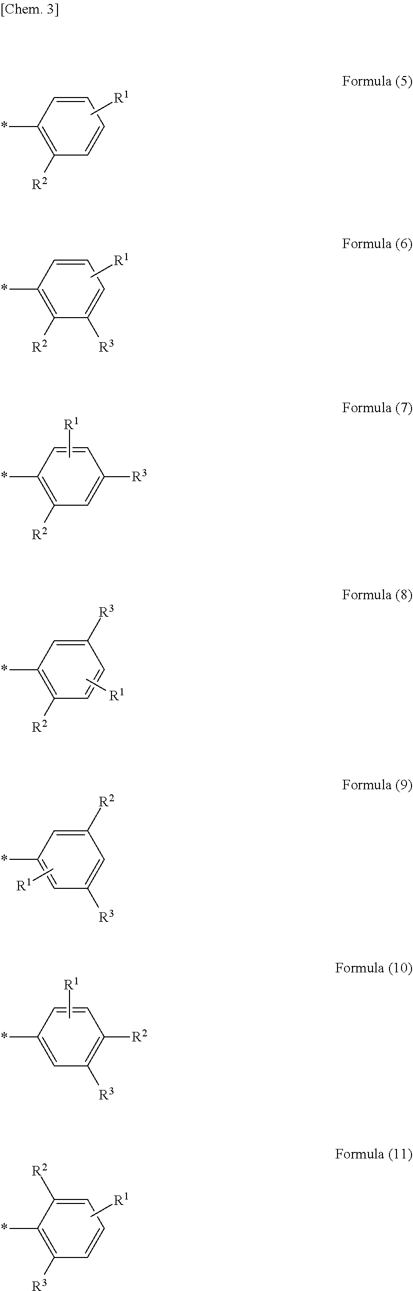

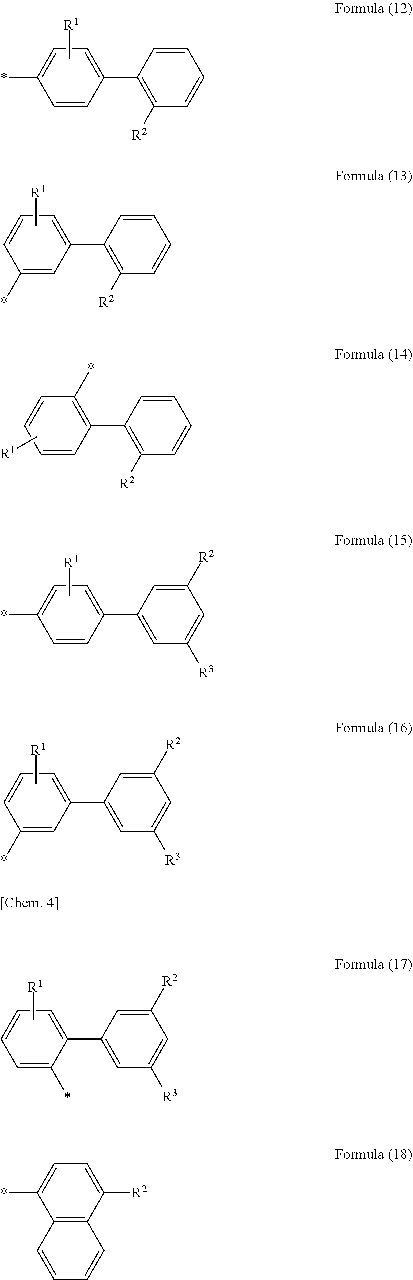

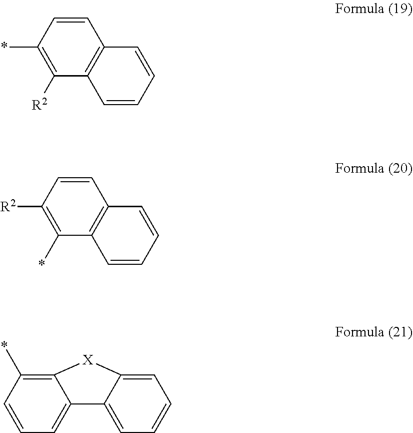

wherein
R¹ denotes a methyl group or a hydrogen atom,
and R² and R³ each independently denote a phenyl group, a biphenylyl group, a naphthyl group, a phenanthryl group, a dibenzofuranyl group or a dibenzothienyl group, and is optionally substituted with a methyl group.
X denotes an oxygen atom or a sulfur atom.

[2] According to another aspect of the present disclosure, there is provided the transverse current suppressing material for an organic electroluminescent element according to [1], wherein Ar¹ denotes a group represented by any one of the formulae (5) to (21).

[3] According to another aspect of the present disclosure, there is provided the transverse current suppressing material for an organic electroluminescent element according to [1], wherein both Ar¹ and Ar² denote a group represented by any one of the formulae (5) to (21).

[4] According to another aspect of the present disclosure, there is provided the transverse current suppressing material for an organic electroluminescent element according to [1], wherein
Ar¹ to Ar³ each independently denote
(i) a phenyl group, a biphenylyl group, a terphenylyl group, a naphthyl group, a fluorenyl group, a spirobifluorenyl group, a benzofluorenyl group, a phenanthryl group, a fluoranthenyl group, a triphenylenyl group, an anthryl group, a pyrenyl group, a dibenzofuranyl group or a dibenzothienyl group,
(ii) the group represented by (i) substituted with one or more groups selected from the group consisting of a methyl group, an ethyl group, a methoxy group, an ethoxy group, a cyano group, a deuterium atom, a fluorine atom, a phenyl group, a biphenylyl group, a naphthyl group, a phenanthryl group, a triphenylsilyl group, a carbazolyl group, a dibenzothienyl group and a dibenzofuranyl group or
(iii) a group represented by any one of the formulae (5) to (21).

[5] According to another aspect of the present disclosure,
the transverse current suppressing material for an organic electroluminescent element according to [1] or [2], wherein at least one of Ar¹ to Ar³ denotes a group represented by any one of the formulae (Y1) to (Y298).

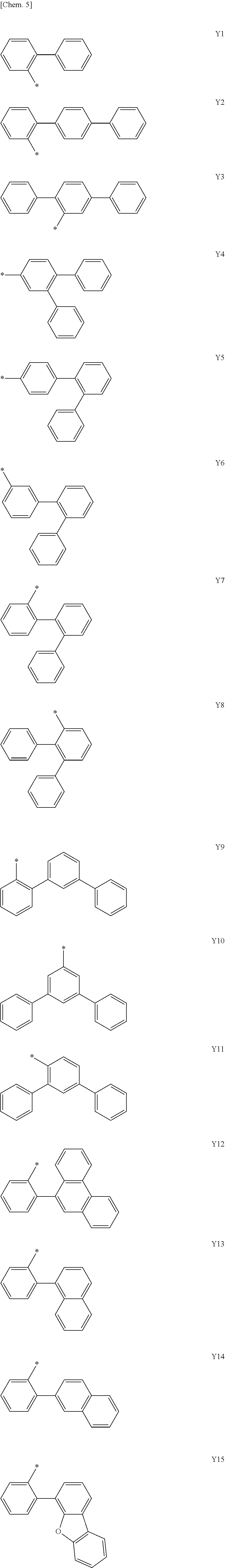

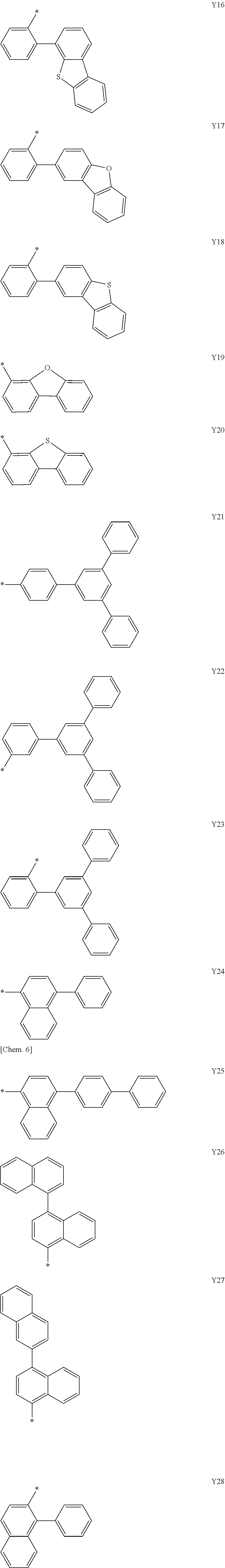

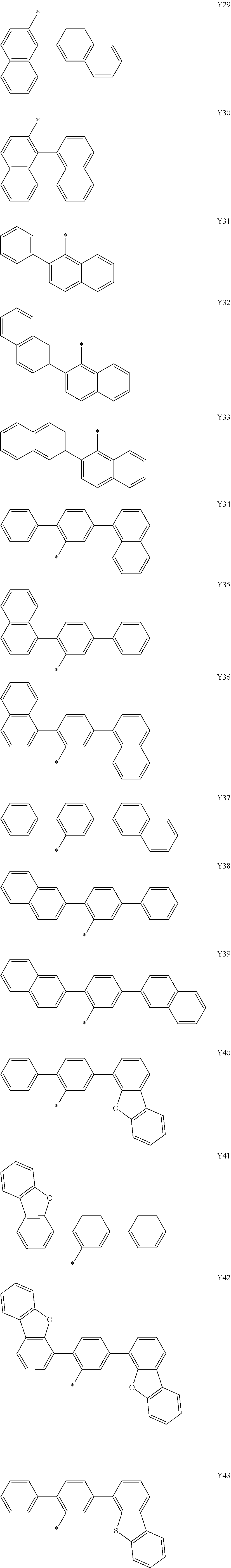

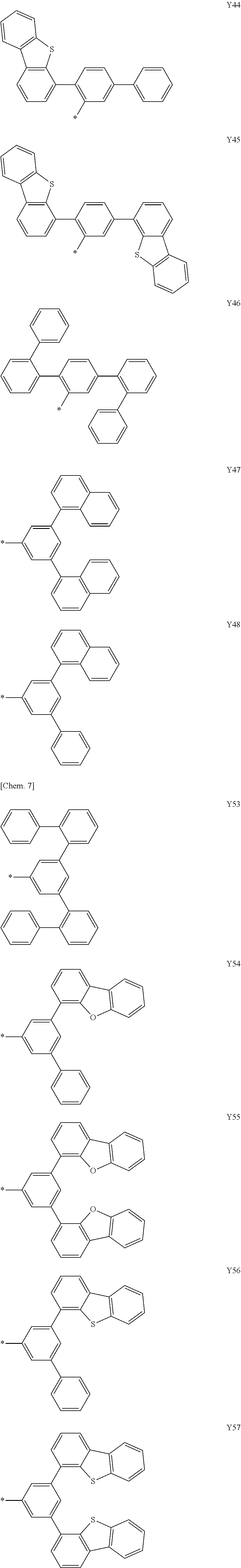

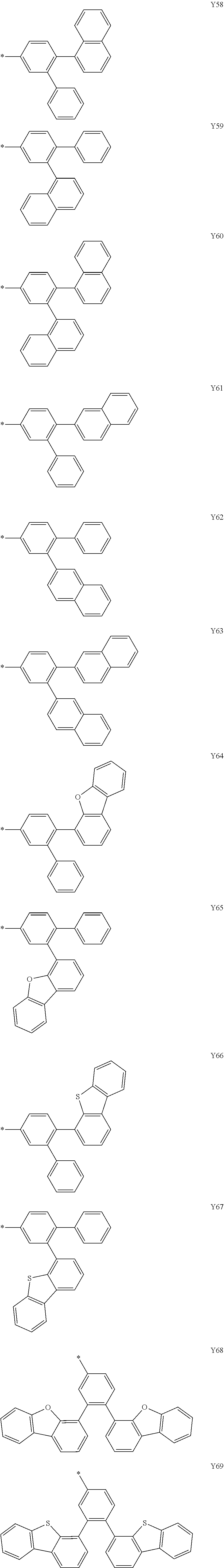

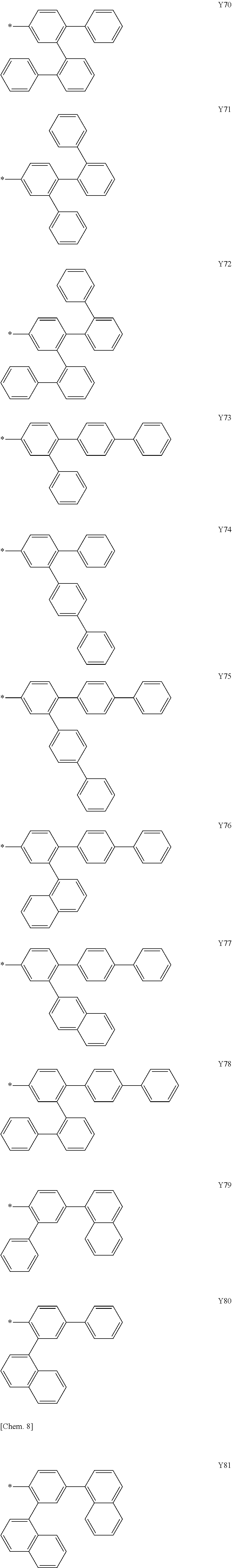

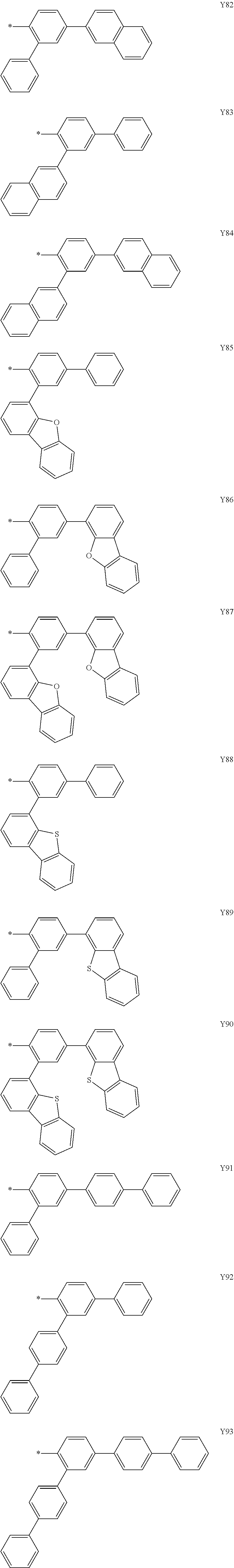

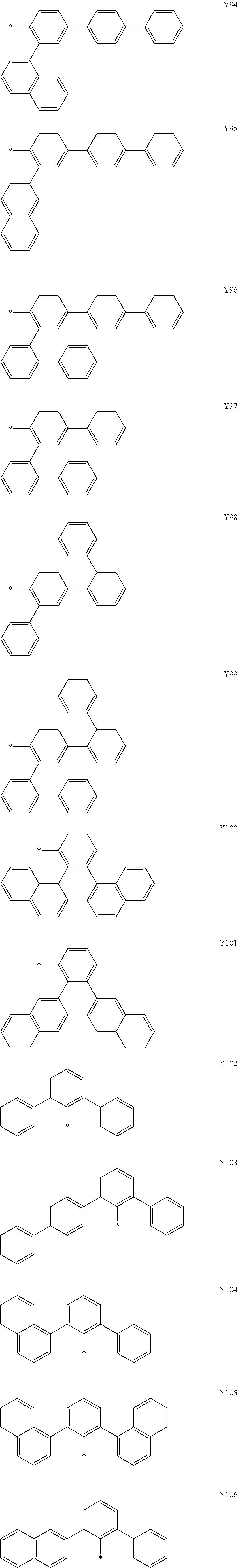

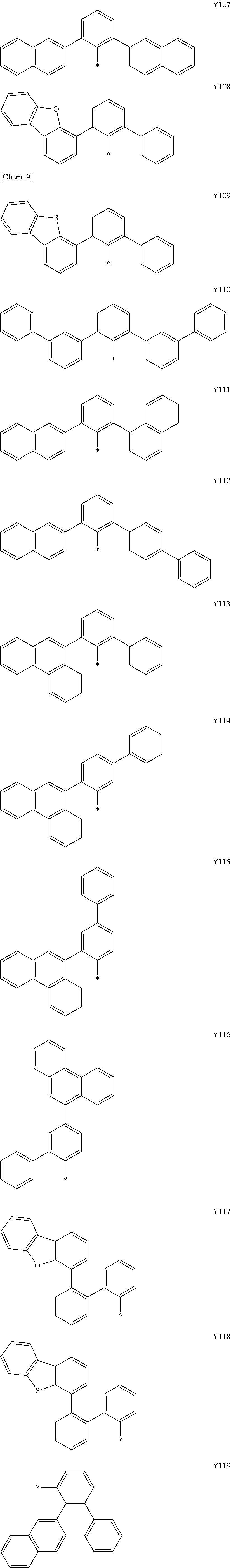

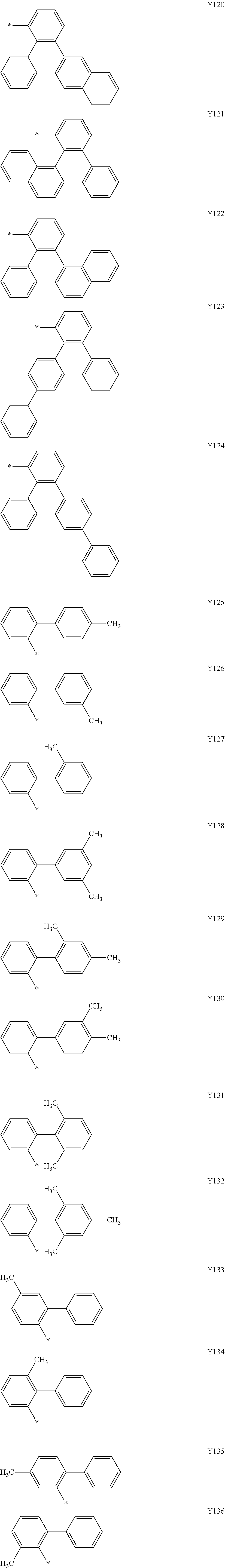

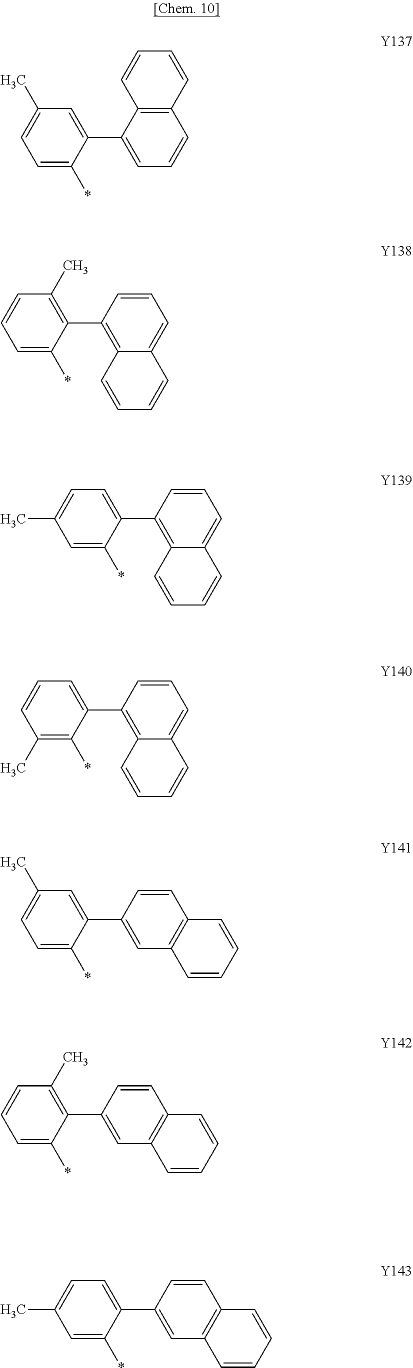

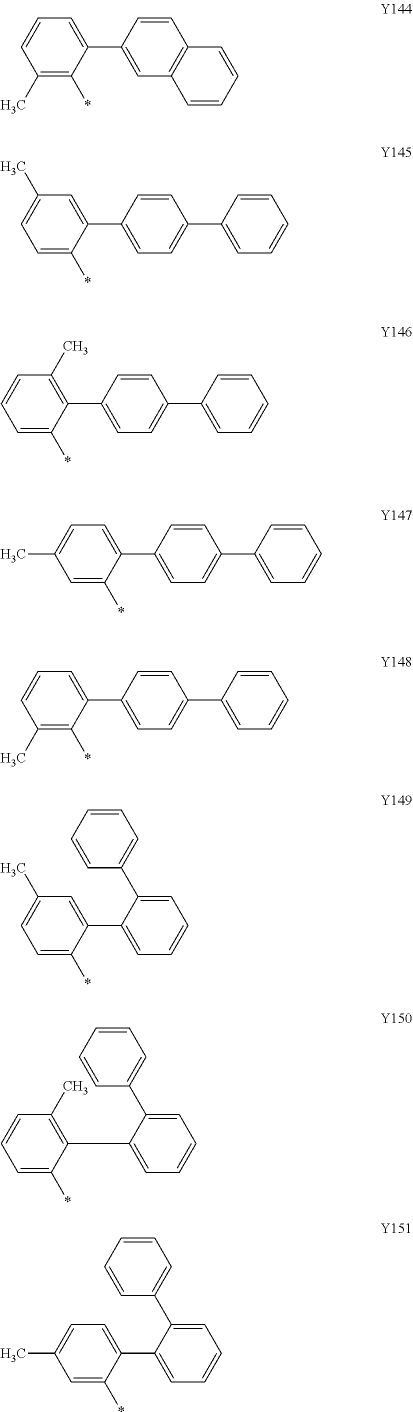

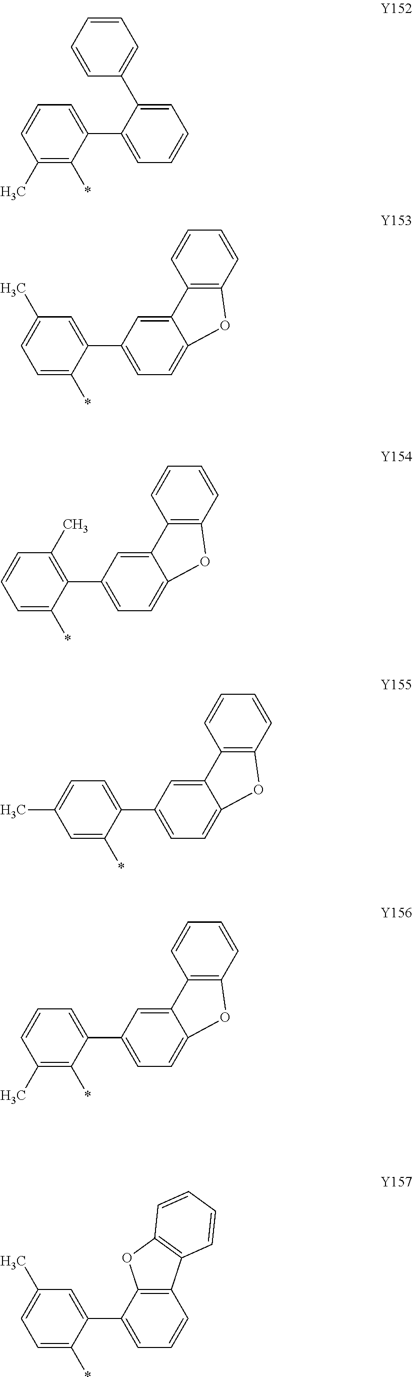 [SMILES: *c1c(C)cccc1-c1ccccc1-c1ccccc1 ; *c1ccc(C)cc1-c1ccc2oc3ccccc3c2c1 ; *c1cccc(C)c1-c1ccc2oc3ccccc3c2c1 ; *c1cc(C)ccc1-c1ccc2oc3ccccc3c2c1 ; *c1c(C)cccc1-c1ccc2oc3ccccc3c2c1 ; *c1ccc(C)cc1-c1cccc2c1oc1ccccc12]

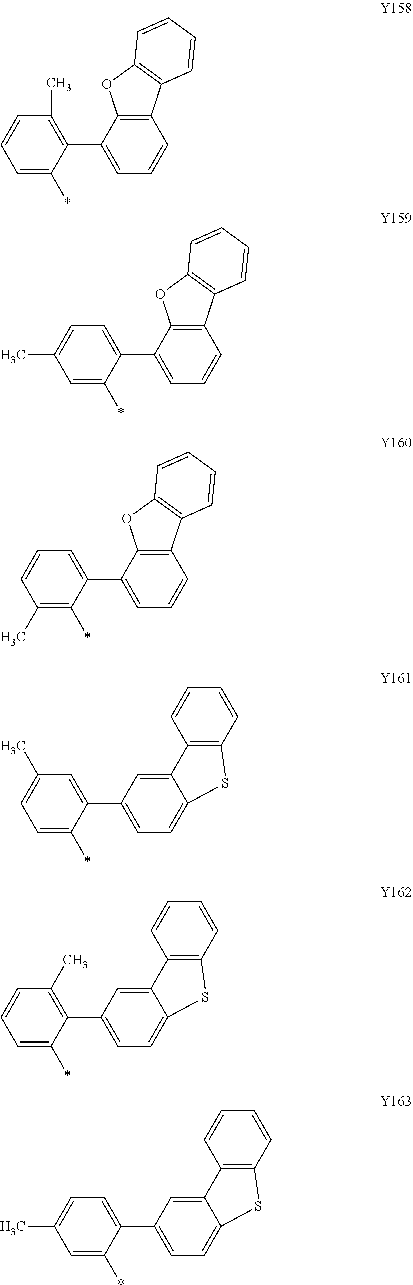 [SMILES: *c1cccc(C)c1-c1cccc2c1oc1ccccc12 ; *c1cc(C)ccc1-c1cccc2c1oc1ccccc12 ; *c1c(C)cccc1-c1cccc2c1oc1ccccc12 ; *c1ccc(C)cc1-c1ccc2sc3ccccc3c2c1 ; *c1cccc(C)c1-c1ccc2sc3ccccc3c2c1 ; *c1cc(C)ccc1-c1ccc2sc3ccccc3c2c1]

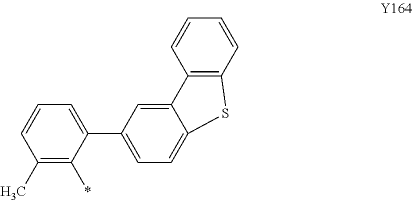 [SMILES: *c1c(C)cccc1-c1ccc2sc3ccccc3c2c1]

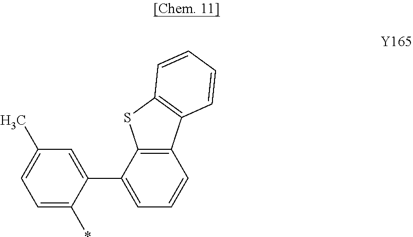 [SMILES: *c1ccc(C)cc1-c1cccc2c1sc1ccccc12]

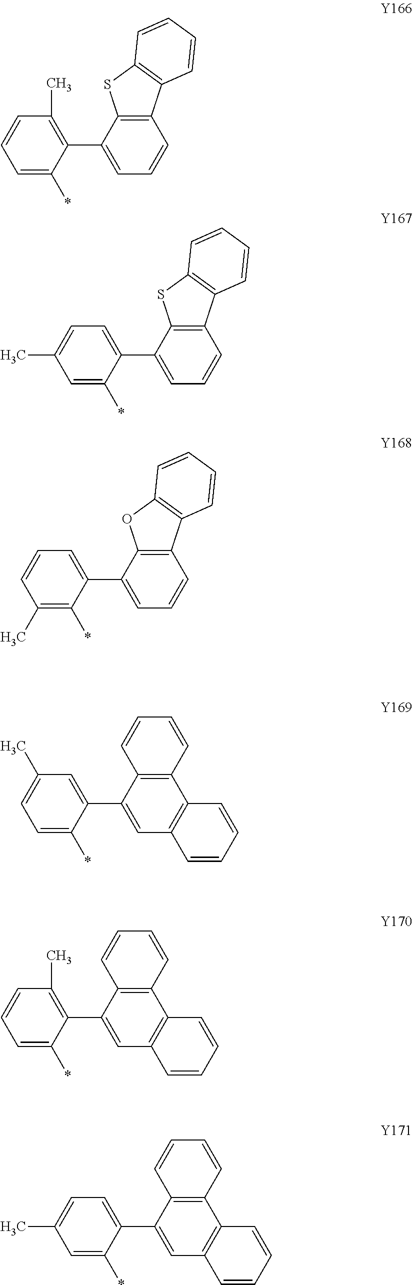 [SMILES: *c1cccc(C)c1-c1cccc2c1sc1ccccc12 ; *c1cc(C)ccc1-c1cccc2c1sc1ccccc12 ; *c1c(C)cccc1-c1cccc2c1oc1ccccc12 ; *c1ccc(C)cc1-c1cc2ccccc2c2ccccc12 ; *c1cccc(C)c1-c1cc2ccccc2c2ccccc12 ; *c1cc(C)ccc1-c1cc2ccccc2c2ccccc12]

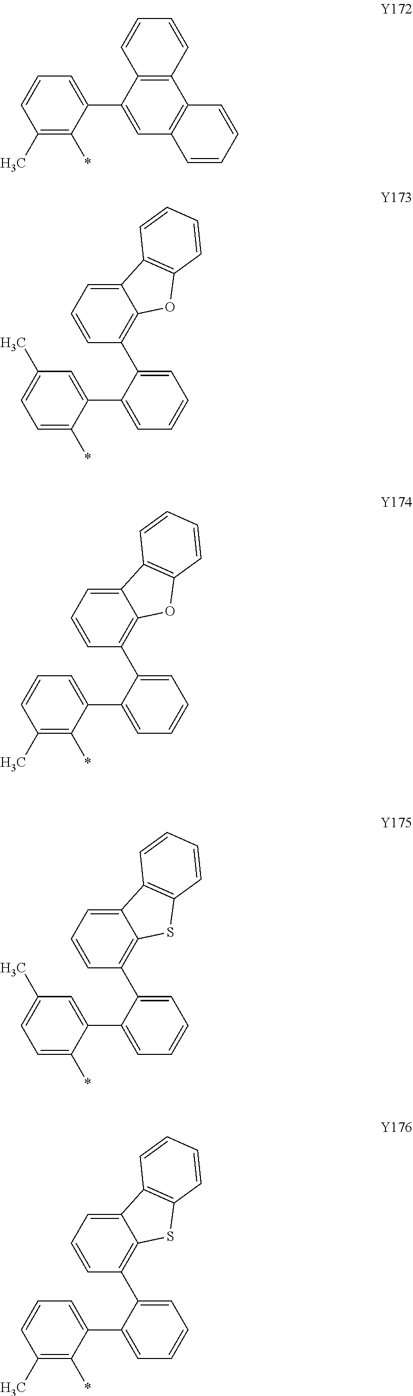 [SMILES: *c1c(C)cccc1-c1cc2ccccc2c2ccccc12 ; *c1ccc(C)cc1-c1ccccc1-c1cccc2c1oc1ccccc12 ; *c1c(C)cccc1-c1ccccc1-c1cccc2c1oc1ccccc12 ; *c1ccc(C)cc1-c1ccccc1-c1cccc2c1sc1ccccc12 ; *c1c(C)cccc1-c1ccccc1-c1cccc2c1sc1ccccc12]

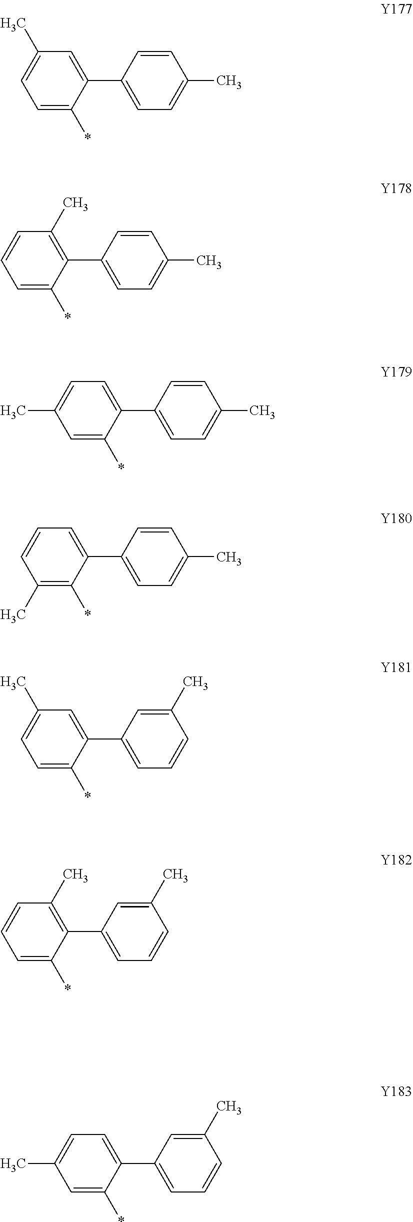

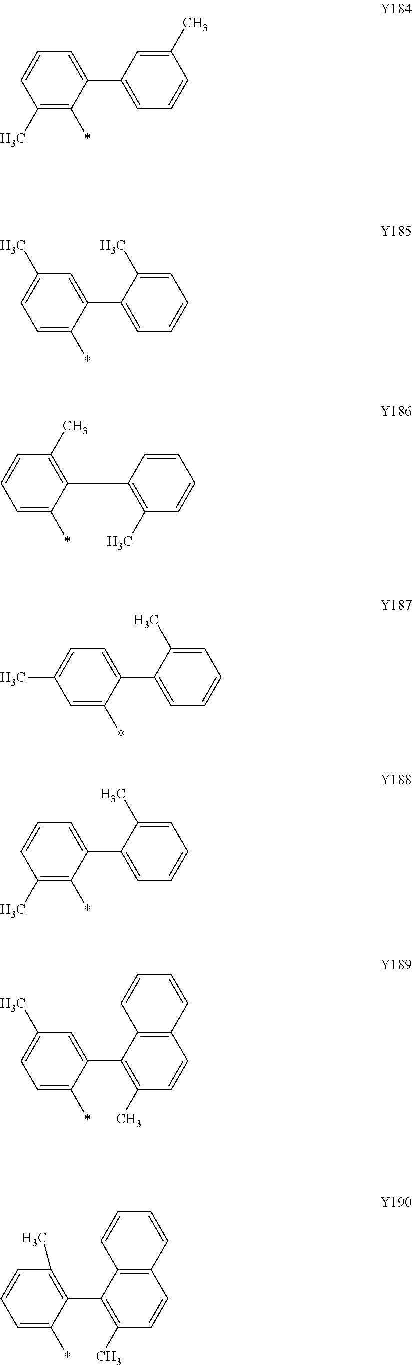

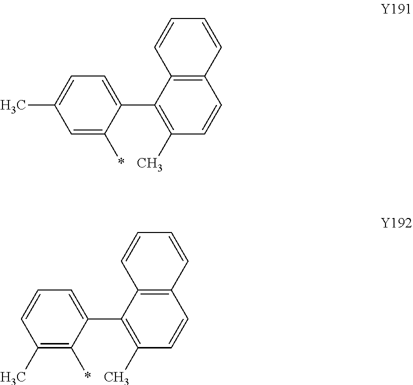 [SMILES: *c1cc(C)ccc1-c1c(C)ccc2ccccc12 ; *c1c(C)cccc1-c1c(C)ccc2ccccc12]

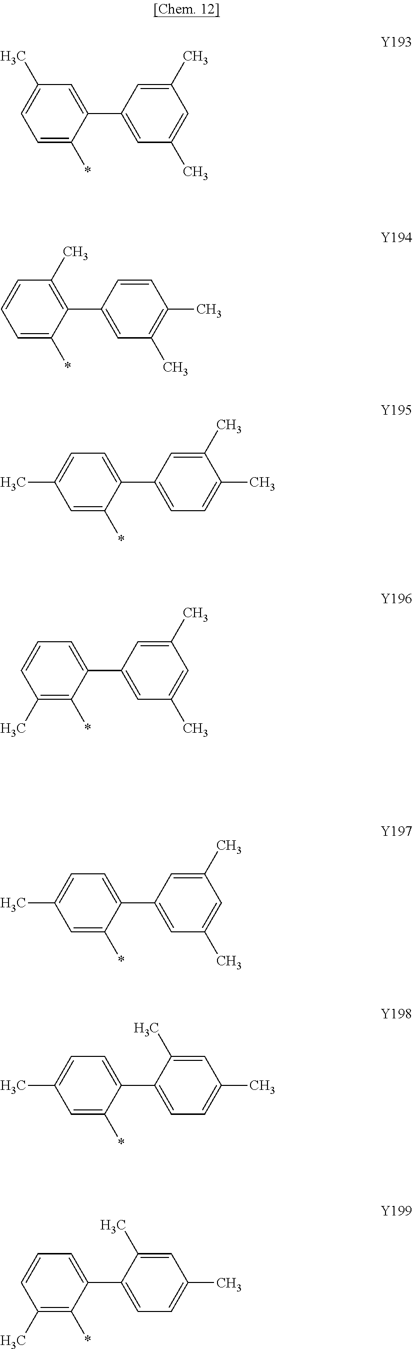

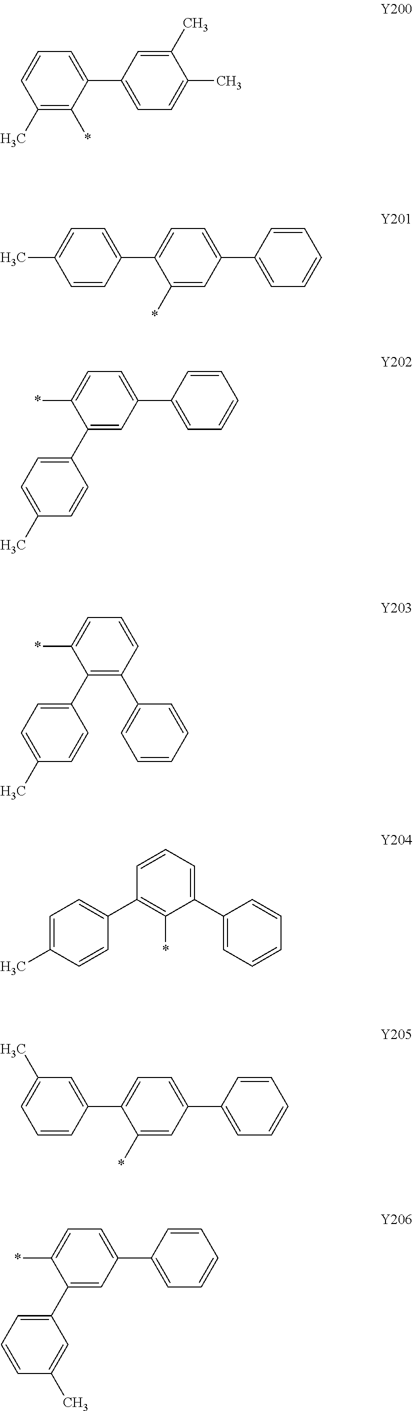

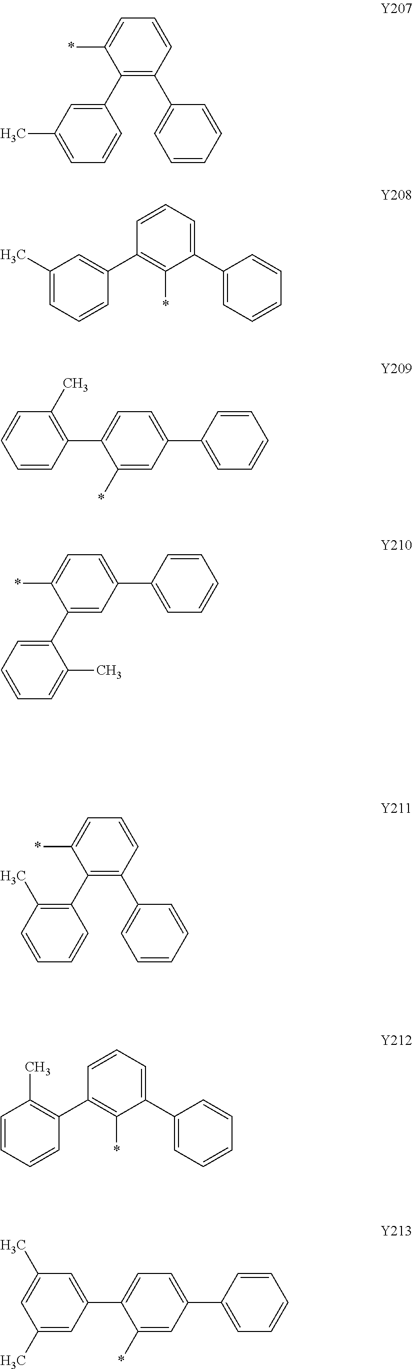

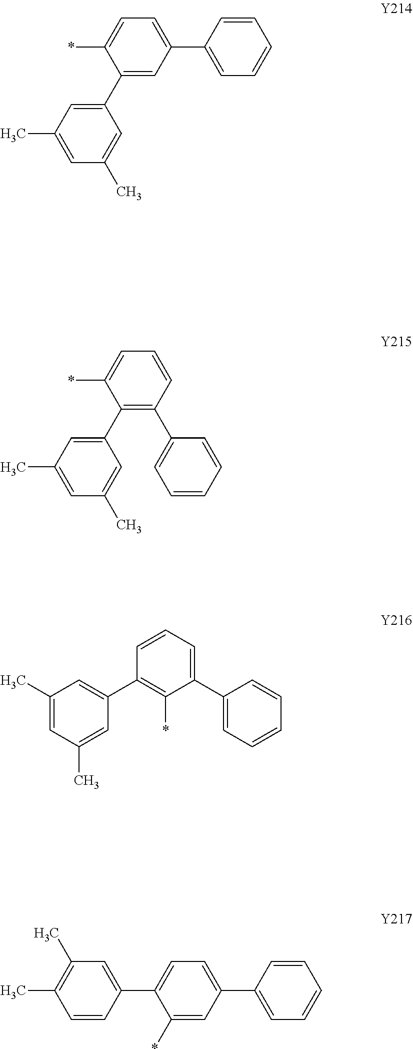 [SMILES: *c1ccc(-c2ccccc2)cc1-c1cc(C)cc(C)c1 ; *c1cccc(-c2ccccc2)c1-c1cc(C)cc(C)c1 ; *c1c(-c2ccccc2)cccc1-c1cc(C)cc(C)c1 ; *c1cc(-c2ccccc2)ccc1-c1ccc(C)c(C)c1]

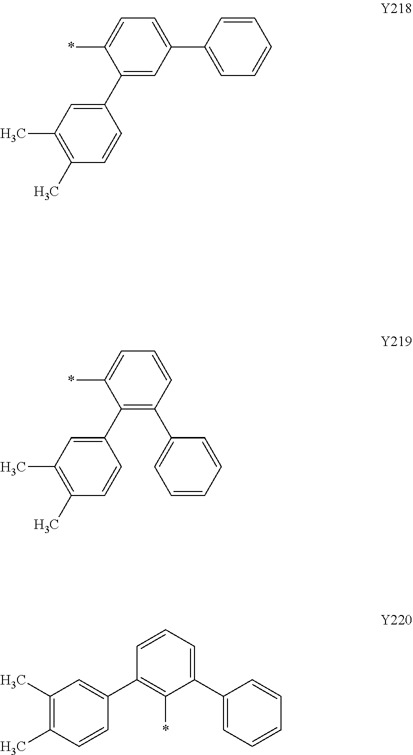 [SMILES: *c1ccc(-c2ccccc2)cc1-c1ccc(C)c(C)c1 ; *c1cccc(-c2ccccc2)c1-c1ccc(C)c(C)c1 ; *c1c(-c2ccccc2)cccc1-c1ccc(C)c(C)c1]

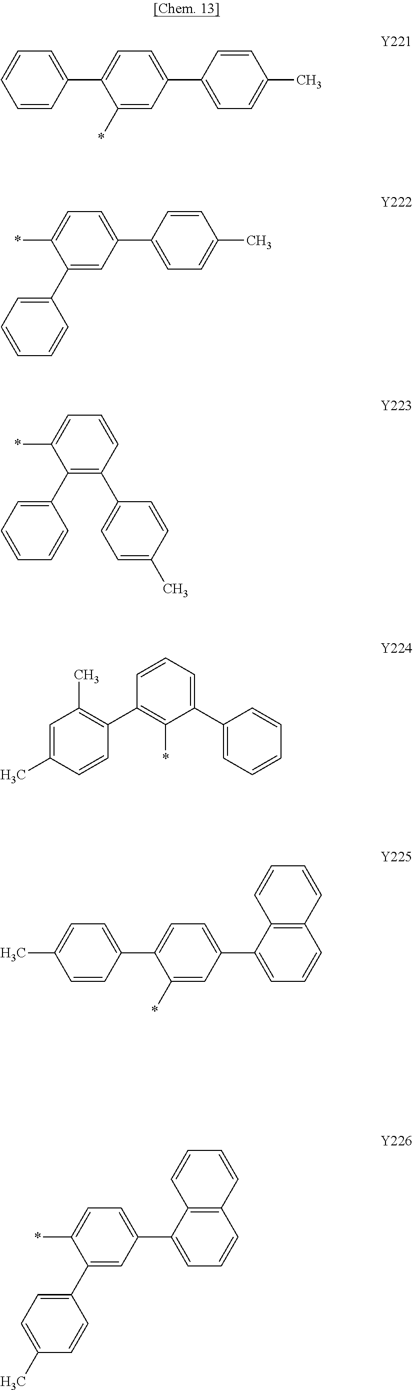

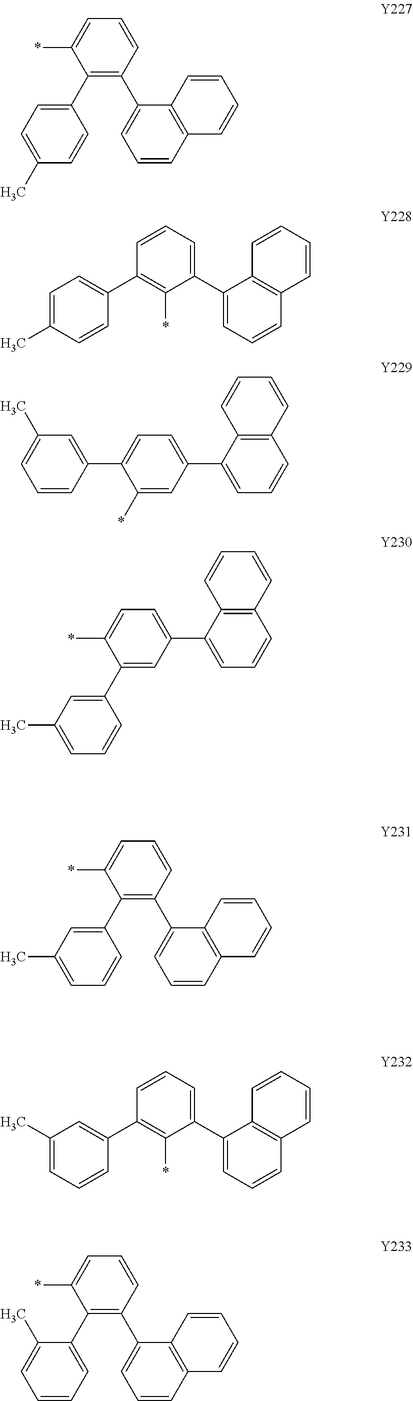

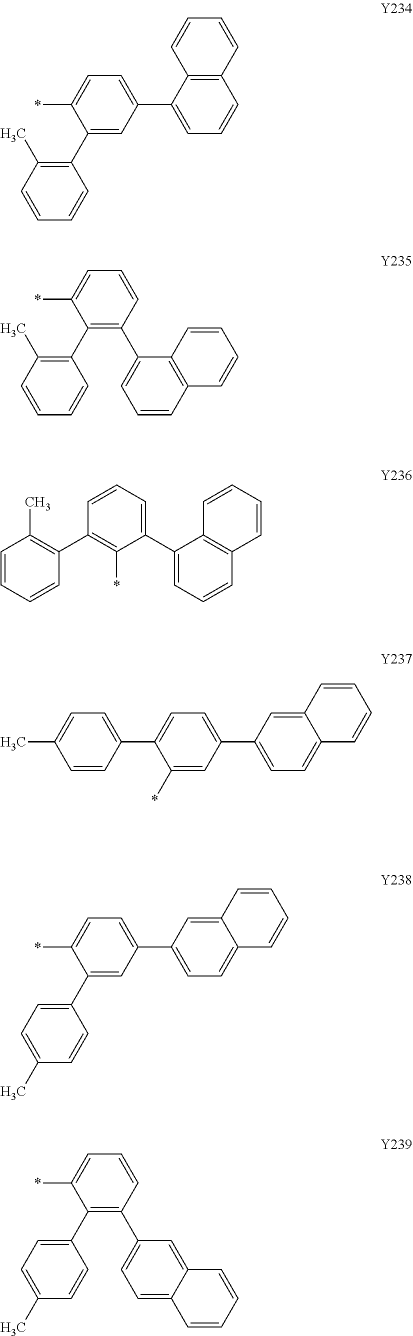 [SMILES: *c1ccc(-c2cccc3ccccc23)cc1-c1ccccc1C ; *c1cccc(-c2cccc3ccccc23)c1-c1ccccc1C ; *c1c(-c2ccccc2C)cccc1-c1cccc2ccccc12 ; *c1cc(-c2ccc3ccccc3c2)ccc1-c1ccc(C)cc1 ; *c1ccc(-c2ccc3ccccc3c2)cc1-c1ccc(C)cc1 ; *c1cccc(-c2ccc3ccccc3c2)c1-c1ccc(C)cc1]

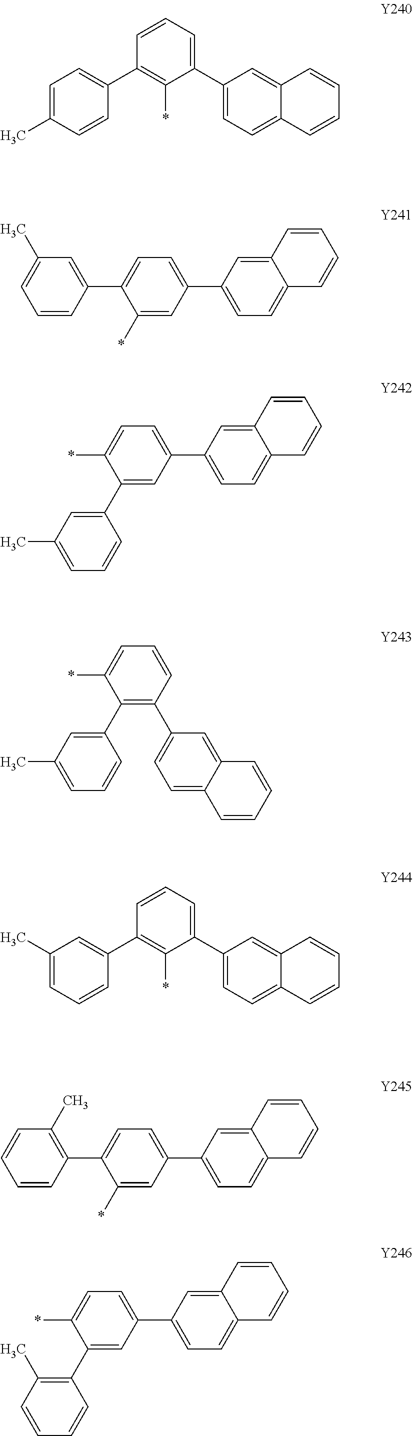

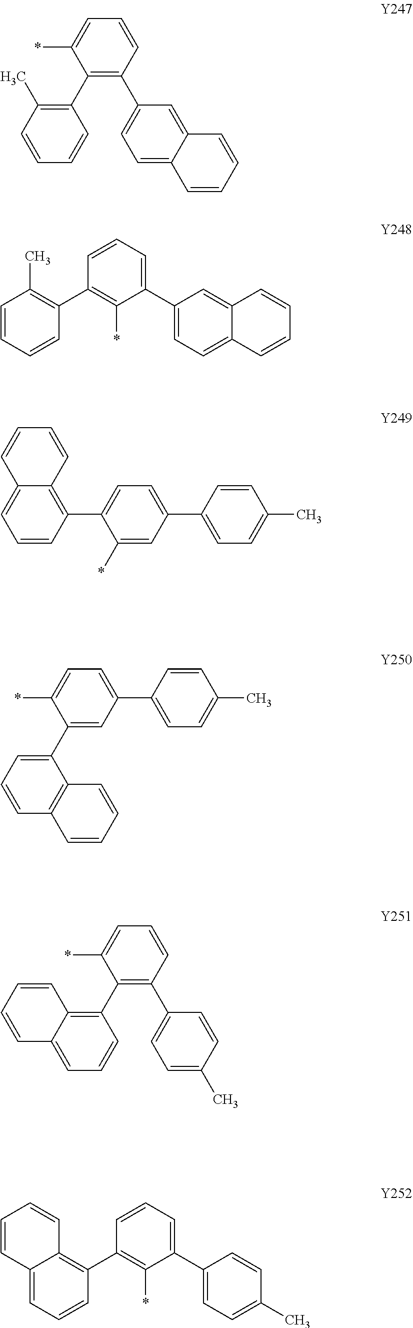 [SMILES: *c1cccc(-c2ccc3ccccc3c2)c1-c1ccccc1C ; *c1c(-c2ccc3ccccc3c2)cccc1-c1ccccc1C ; *c1cc(-c2ccc(C)cc2)ccc1-c1cccc2ccccc12 ; *c1ccc(-c2ccc(C)cc2)cc1-c1cccc2ccccc12 ; *c1cccc(-c2ccc(C)cc2)c1-c1cccc2ccccc12 ; *c1c(-c2ccc(C)cc2)cccc1-c1cccc2ccccc12]

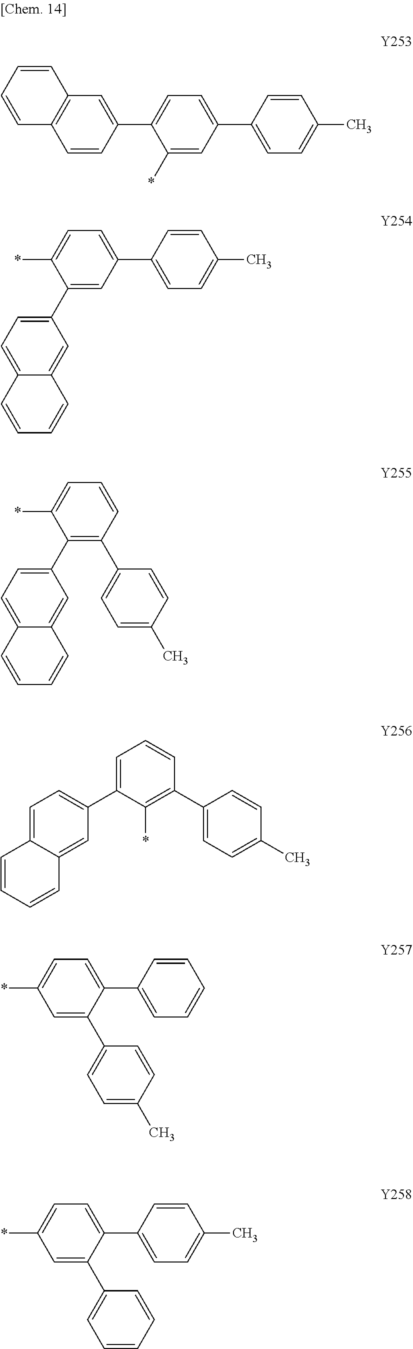 [SMILES: *c1cc(-c2ccc(C)cc2)ccc1-c1ccc2ccccc2c1 ; *c1ccc(-c2ccc(C)cc2)cc1-c1ccc2ccccc2c1 ; *c1cccc(-c2ccc(C)cc2)c1-c1ccc2ccccc2c1 ; *c1c(-c2ccc(C)cc2)cccc1-c1ccc2ccccc2c1 ; *c1ccc(-c2ccccc2)c(-c2ccc(C)cc2)c1 ; *c1ccc(-c2ccc(C)cc2)c(-c2ccccc2)c1]

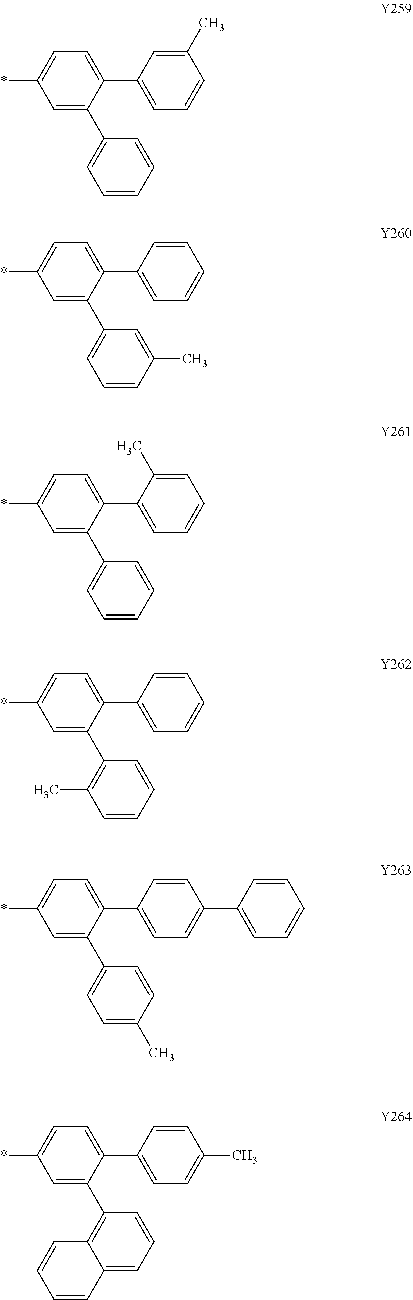 [SMILES: *c1ccc(-c2cccc(C)c2)c(-c2ccccc2)c1 ; *c1ccc(-c2ccccc2)c(-c2cccc(C)c2)c1 ; *c1ccc(-c2ccccc2C)c(-c2ccccc2)c1 ; *c1ccc(-c2ccccc2)c(-c2ccccc2C)c1 ; *c1ccc(-c2ccc(-c3ccccc3)cc2)c(-c2ccc(C)cc2)c1 ; *c1ccc(-c2ccc(C)cc2)c(-c2cccc3ccccc23)c1]

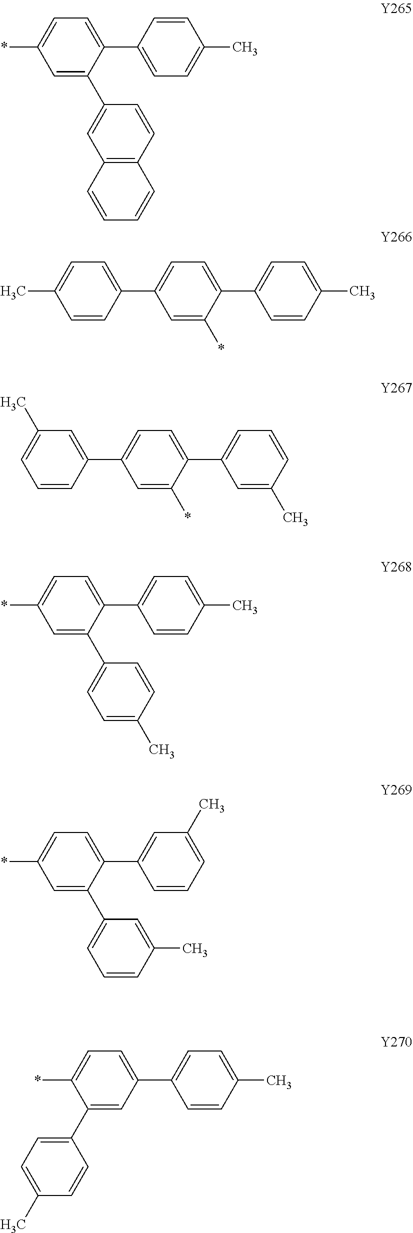

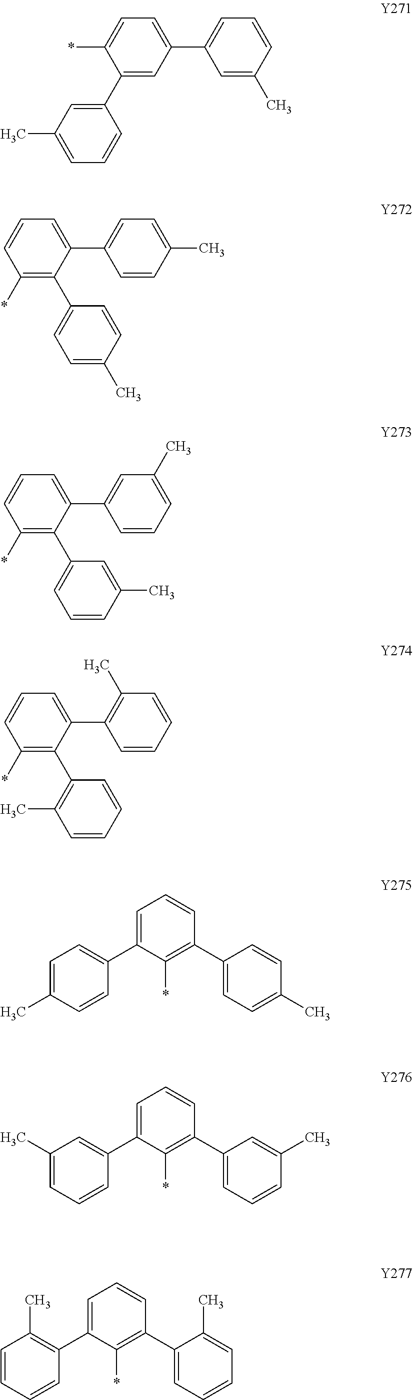

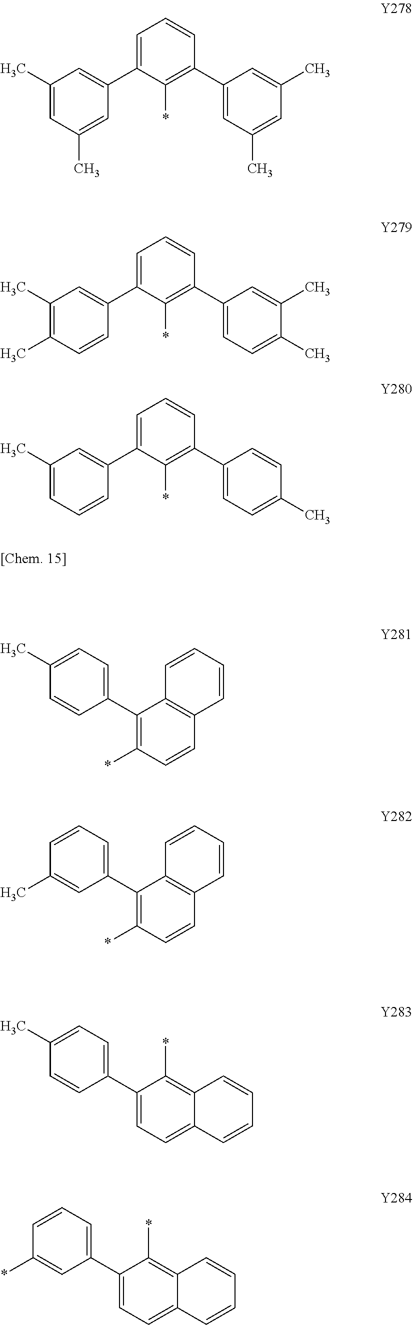

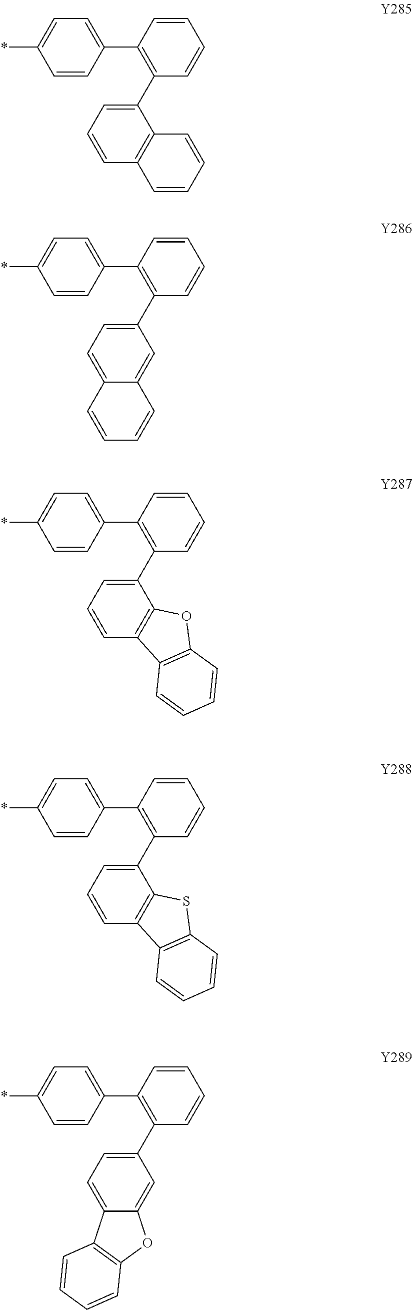

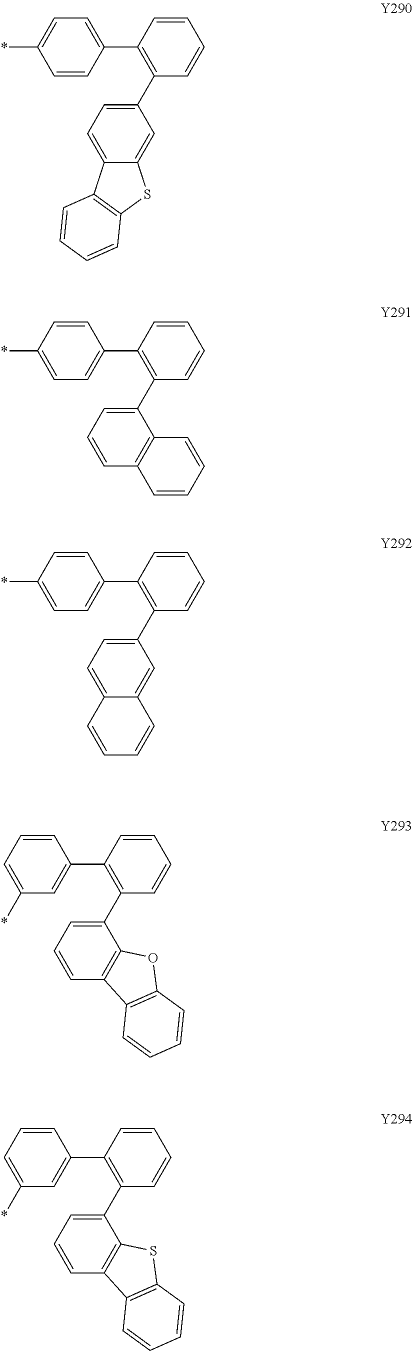 [SMILES: *c1ccc(-c2ccccc2-c2ccc3c(c2)sc2ccccc23)cc1 ; *c1ccc(-c2ccccc2-c2cccc3ccccc23)cc1 ; *c1ccc(-c2ccccc2-c2ccc3ccccc3c2)cc1 ; *c1cccc(-c2ccccc2-c2cccc3c2oc2ccccc23)c1 ; *c1cccc(-c2ccccc2-c2cccc3c2sc2ccccc23)c1]

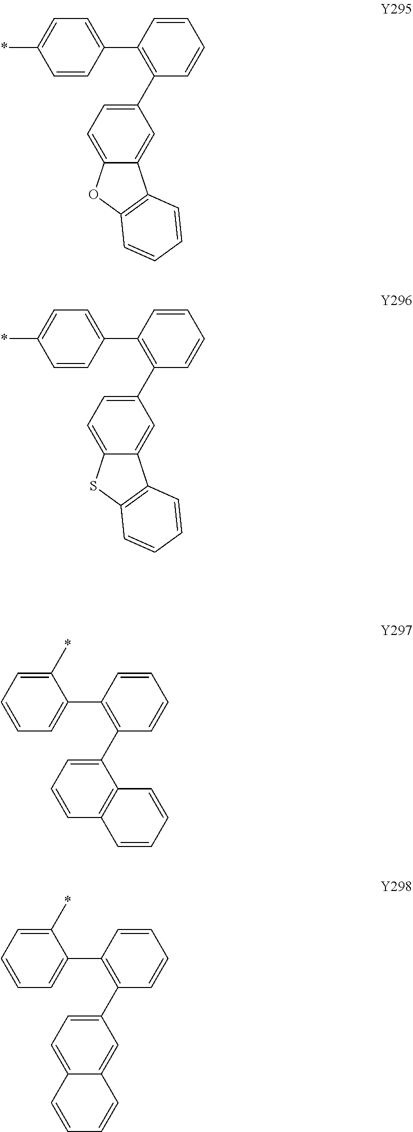 [SMILES: *c1ccc(-c2ccccc2-c2ccc3oc4ccccc4c3c2)cc1 ; *c1ccc(-c2ccccc2-c2ccc3sc4ccccc4c3c2)cc1 ; *c1ccccc1-c1ccccc1-c1cccc2ccccc12 ; *c1ccccc1-c1ccccc1-c1ccc2ccccc2c1]

[6] According to another aspect of the present disclosure, there is provided the transverse current suppressing material for an organic electroluminescent element according to [1], wherein both Ar¹ and Ar² each independently denote a group represented by any one of the formulae (Y2) to (Y9), (Y11) to (Y18) and (Y21) to (Y298).

[7] According to another aspect of the present disclosure,
there is provided a carbazole compound represented by the formula (22) or (23):

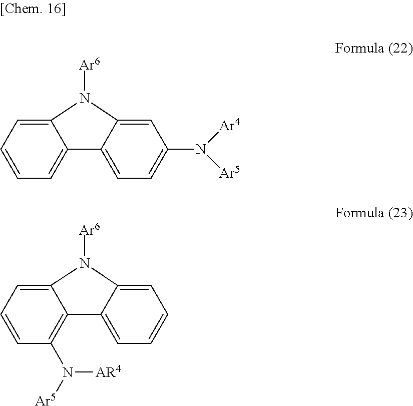

wherein
Ar⁶ each independently denotes a group selected from the formulae (24) to (45),

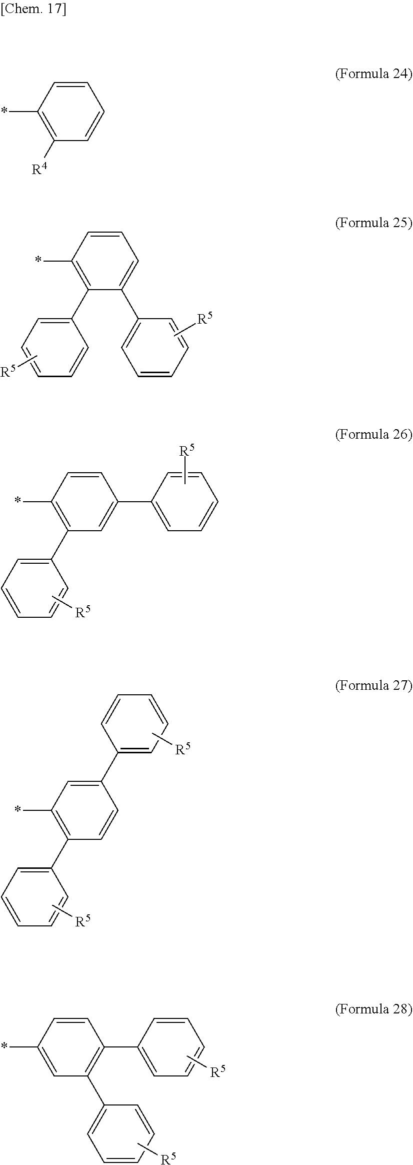

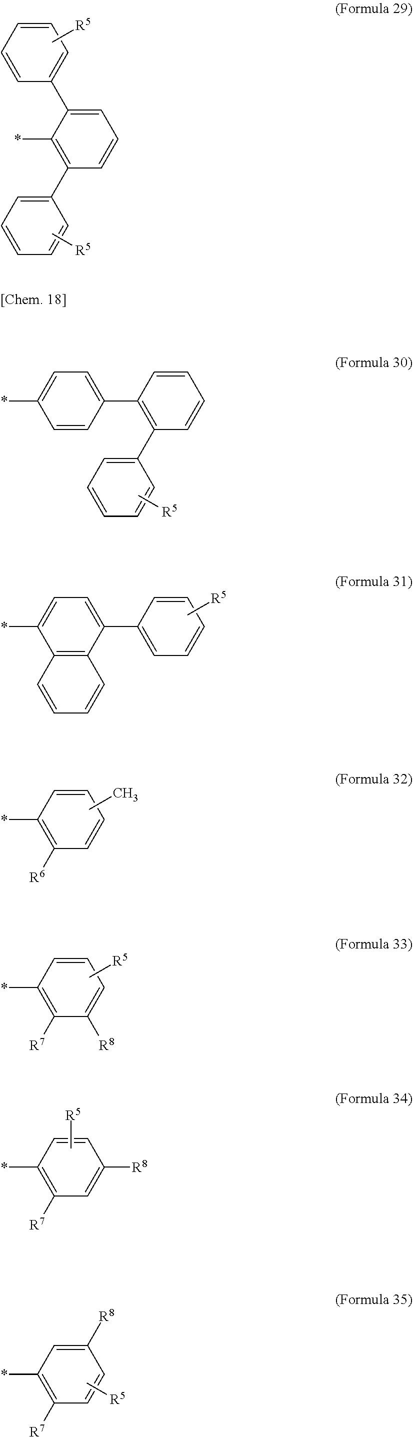

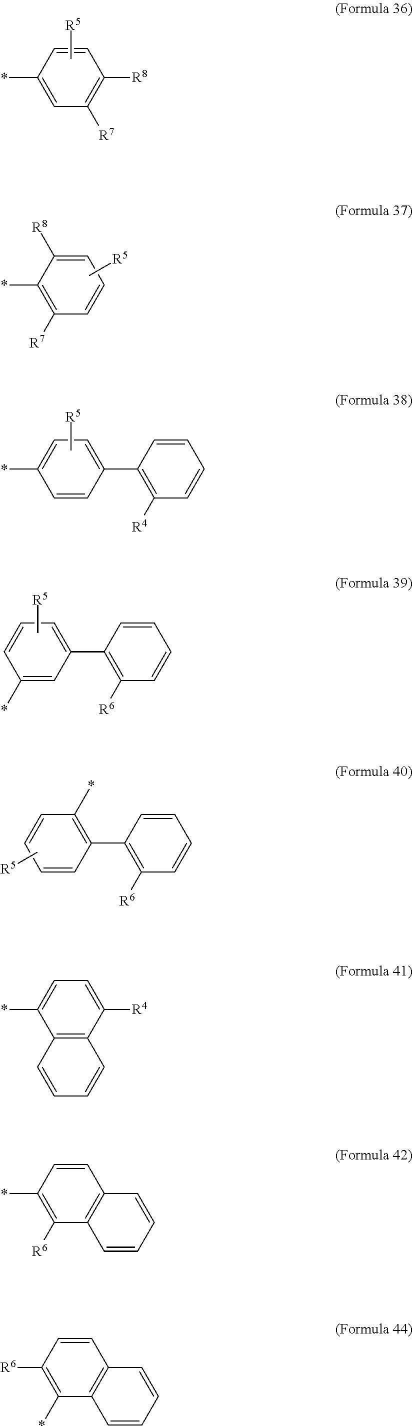

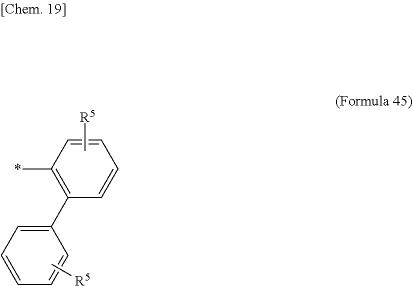

wherein
R⁴ denotes a biphenylyl group, a naphthyl group, a phenanthryl group, a dibenzofuranyl group or a dibenzothienyl group each optionally substituted with a methyl group,
R⁵ each independently denotes a methyl group or a hydrogen atom,
R⁶ denotes a phenyl group, a biphenylyl group, a naphthyl group, a phenanthryl group, a dibenzofuranyl group or a dibenzothienyl group each optionally substituted with a methyl group,
R⁷ and R⁸ each independently denote a phenyl group, a biphenylyl group, a naphthyl group, a phenanthryl group, a dibenzofuranyl group or a dibenzothienyl group each optionally substituted with a methyl group, and at least one of R⁷ and R⁸ denotes a biphenylyl group, a naphthyl group, a phenanthryl group, a dibenzofuranyl group or a dibenzothienyl group each optionally substituted with a methyl group,
in the formulae (22) and (23), when Ar⁶ denotes a group selected from the formulae (24) to (31),
Ar⁵ denotes a group selected from the formulae (24) to (45), and Ar⁴ denotes an optionally substituted monocyclic, linked or fused aromatic hydrocarbon group with 6 to 30 carbon atoms or a group represented by an optionally substituted monocyclic, linked or fused heteroaromatic group with 3 to 30 carbon atoms, and
in the formulae (22) and (23), when Ar⁶ denotes a group selected from the formulae (32) to (44),
Ar⁴ and Ar⁵ each independently denote a group selected from the formulae (24) to (45), an optionally substituted monocyclic, linked or fused aromatic hydrocarbon group with 6 to 30 carbon atoms or a group represented by an optionally substituted monocyclic, linked or fused heteroaromatic group with 3 to 30 carbon atoms.

[8] According to another aspect of the present disclosure,
there is provided the carbazole compound according to [7], wherein
Ar⁴ denotes
(iv) a phenyl group, a biphenylyl group, a terphenylyl group, a naphthyl group, a fluorenyl group, a spirobifluorenyl group, a benzofluorenyl group, a phenanthryl group, a fluoranthenyl group, a triphenylenyl group, an anthryl group, a pyrenyl group, a dibenzofuranyl group or a dibenzothienyl group,
(v) the group represented by (iv) substituted with one or more groups selected from the group consisting of a methyl group, an ethyl group, a methoxy group, an ethoxy group, a cyano group, a deuterium atom, a fluorine atom, a phenyl group, a biphenylyl group, a naphthyl group, a phenanthryl group, a triphenylsilyl group, a carbazolyl group, a dibenzothienyl group and a dibenzofuranyl group or
(vi) a group represented by any one of the formulae (24) to (41).

[9] According to another aspect of the present disclosure,
there is provided the carbazole compound according to [7] or [8], wherein Ar⁶ denotes a group represented by (Z1) to (Z209).

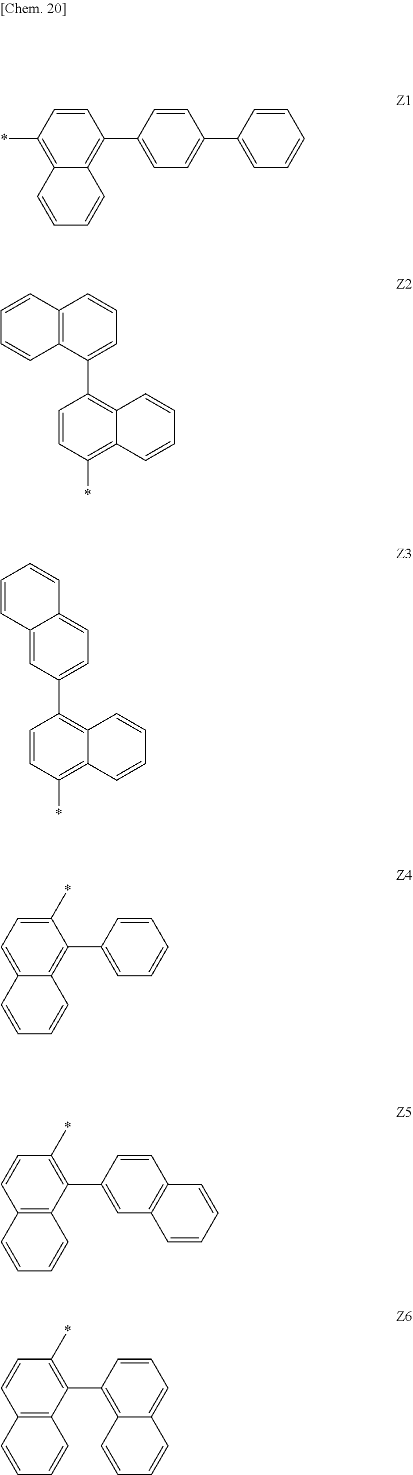 [SMILES: *c1ccc(-c2ccc(-c3ccccc3)cc2)c2ccccc12 ; *c1ccc(-c2cccc3ccccc23)c2ccccc12 ; *c1ccc(-c2ccc3ccccc3c2)c2ccccc12 ; *c1ccc2ccccc2c1-c1ccccc1 ; *c1ccc2ccccc2c1-c1ccc2ccccc2c1 ; *c1ccc2ccccc2c1-c1cccc2ccccc12]

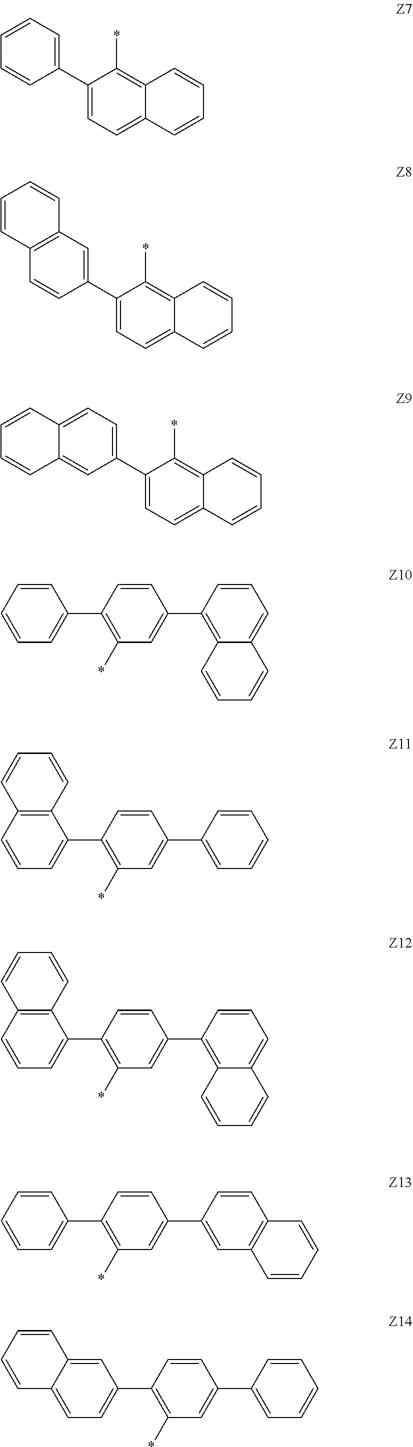

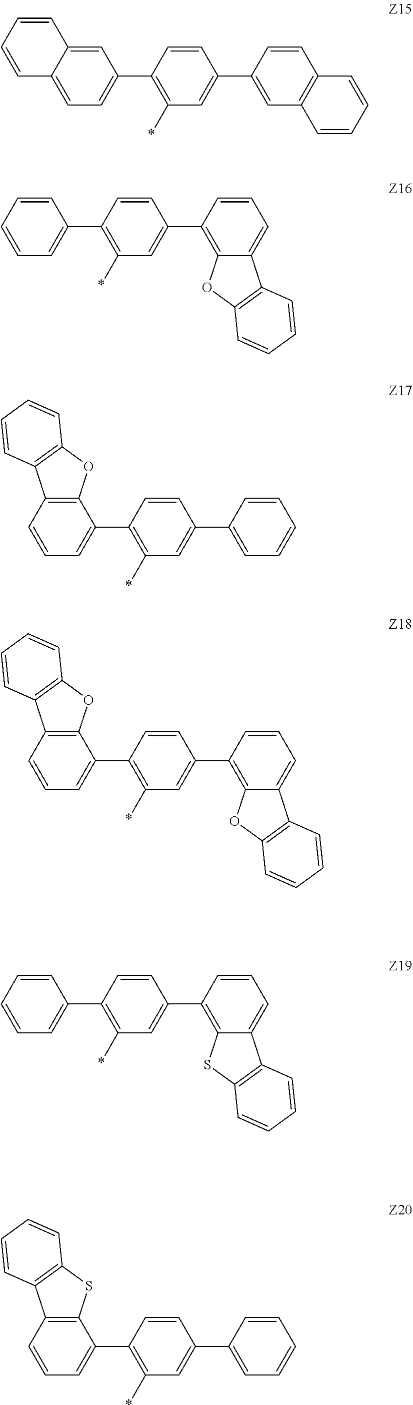 [SMILES: *c1cc(-c2ccc3ccccc3c2)ccc1-c1ccc2ccccc2c1 ; *c1cc(-c2cccc3c2oc2ccccc23)ccc1-c1ccccc1 ; *c1cc(-c2ccccc2)ccc1-c1cccc2c1oc1ccccc12 ; *c1cc(-c2cccc3c2oc2ccccc23)ccc1-c1cccc2c1oc1ccccc12 ; *c1cc(-c2cccc3c2sc2ccccc23)ccc1-c1ccccc1 ; *c1cc(-c2ccccc2)ccc1-c1cccc2c1sc1ccccc12]

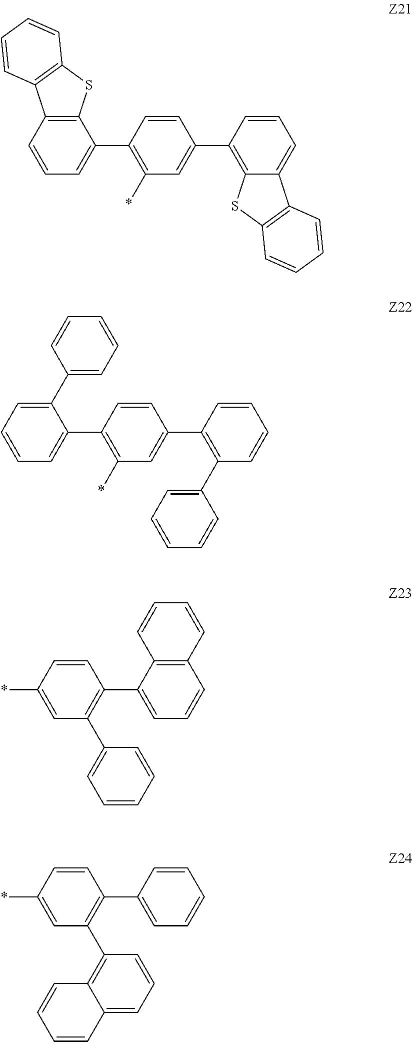 [SMILES: *c1cc(-c2cccc3c2sc2ccccc23)ccc1-c1cccc2c1sc1ccccc12 ; *c1cc(-c2ccccc2-c2ccccc2)ccc1-c1ccccc1-c1ccccc1 ; *c1ccc(-c2cccc3ccccc23)c(-c2ccccc2)c1 ; *c1ccc(-c2ccccc2)c(-c2cccc3ccccc23)c1]

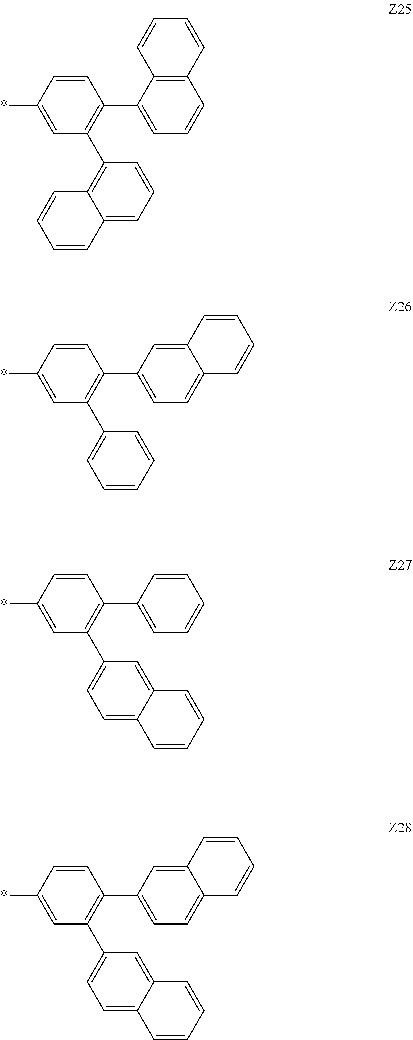 [SMILES: *c1ccc(-c2cccc3ccccc23)c(-c2cccc3ccccc23)c1 ; *c1ccc(-c2ccc3ccccc3c2)c(-c2ccccc2)c1 ; *c1ccc(-c2ccccc2)c(-c2ccc3ccccc3c2)c1 ; *c1ccc(-c2ccc3ccccc3c2)c(-c2ccc3ccccc3c2)c1]

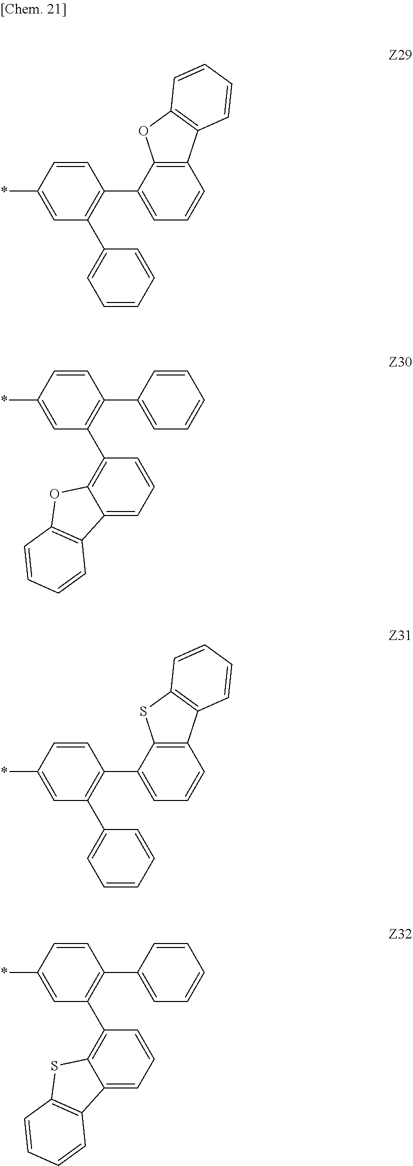 [SMILES: *c1ccc(-c2cccc3c2oc2ccccc23)c(-c2ccccc2)c1 ; *c1ccc(-c2ccccc2)c(-c2cccc3c2oc2ccccc23)c1 ; *c1ccc(-c2cccc3c2sc2ccccc23)c(-c2ccccc2)c1 ; *c1ccc(-c2ccccc2)c(-c2cccc3c2sc2ccccc23)c1]

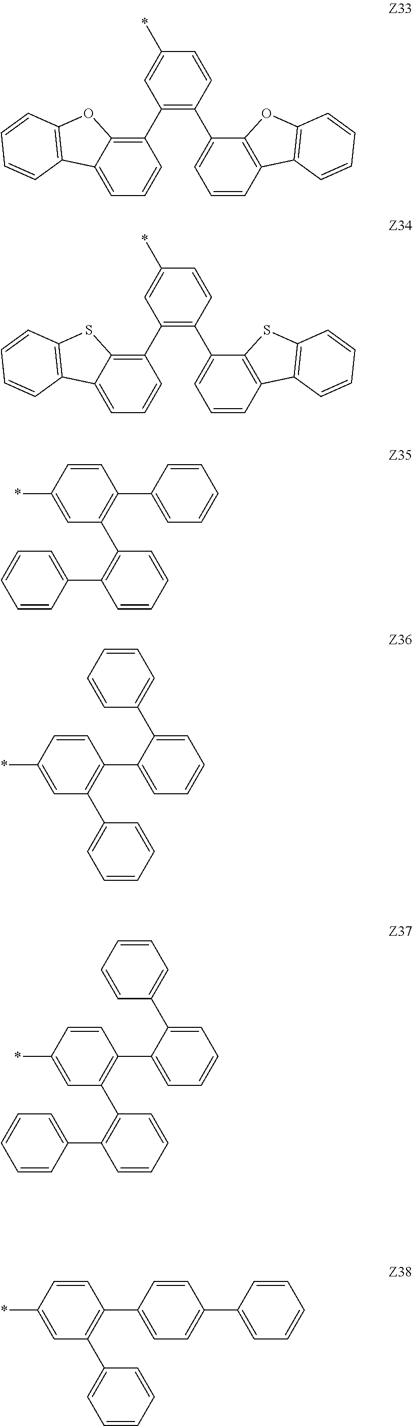

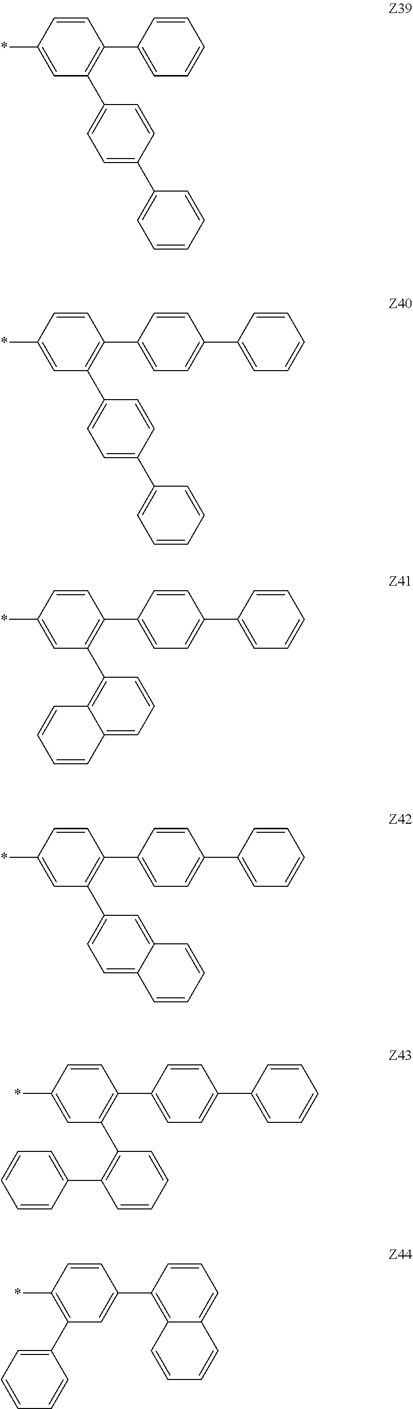

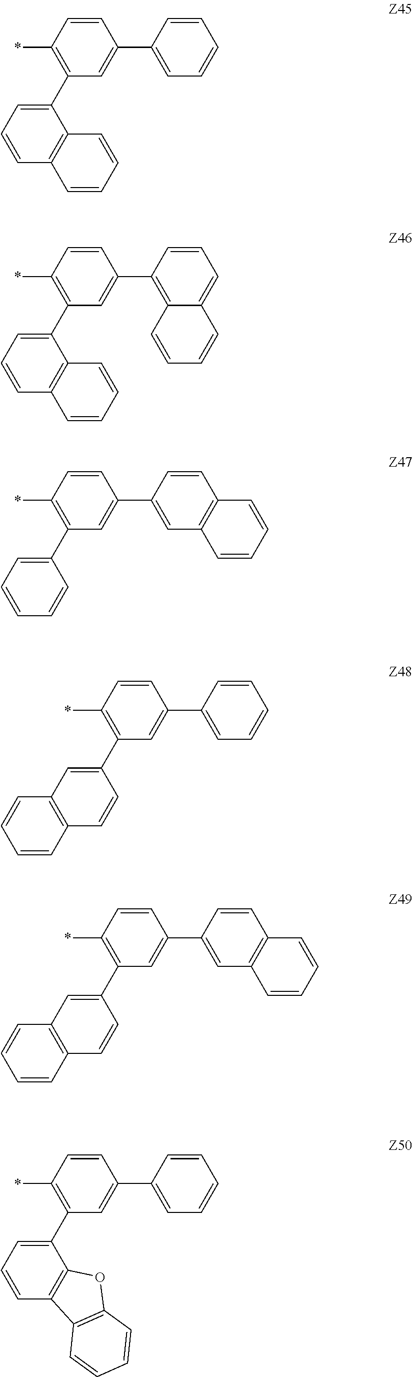 [SMILES: *c1ccc(-c2ccccc2)cc1-c1cccc2ccccc12 ; *c1ccc(-c2cccc3ccccc23)cc1-c1cccc2ccccc12 ; *c1ccc(-c2ccc3ccccc3c2)cc1-c1ccccc1 ; *c1ccc(-c2ccccc2)cc1-c1ccc2ccccc2c1 ; *c1ccc(-c2ccc3ccccc3c2)cc1-c1ccc2ccccc2c1 ; *c1ccc(-c2ccccc2)cc1-c1cccc2c1oc1ccccc12]

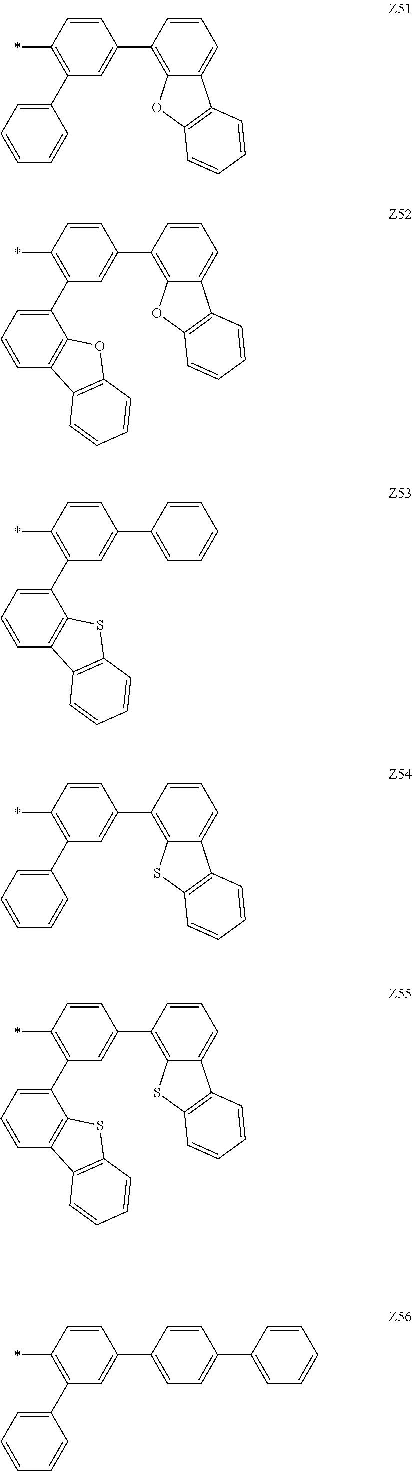 [SMILES: *c1ccc(-c2cccc3c2oc2ccccc23)cc1-c1ccccc1 ; *c1ccc(-c2cccc3c2oc2ccccc23)cc1-c1cccc2c1oc1ccccc12 ; *c1ccc(-c2ccccc2)cc1-c1cccc2c1sc1ccccc12 ; *c1ccc(-c2cccc3c2sc2ccccc23)cc1-c1ccccc1 ; *c1ccc(-c2cccc3c2sc2ccccc23)cc1-c1cccc2c1sc1ccccc12 ; *c1ccc(-c2ccc(-c3ccccc3)cc2)cc1-c1ccccc1]

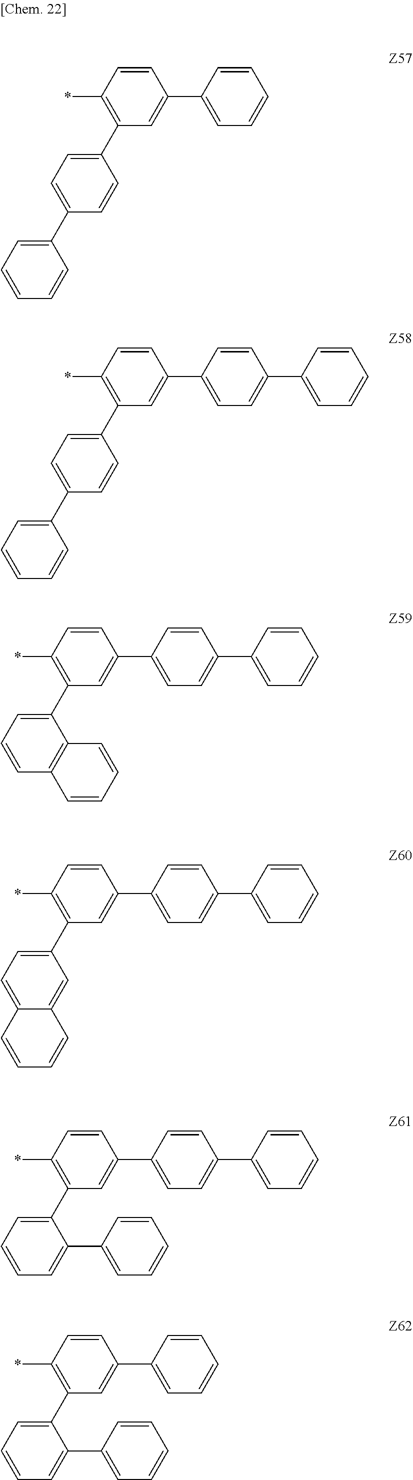 [SMILES: *c1ccc(-c2ccccc2)cc1-c1ccc(-c2ccccc2)cc1 ; *c1ccc(-c2ccc(-c3ccccc3)cc2)cc1-c1ccc(-c2ccccc2)cc1 ; *c1ccc(-c2ccc(-c3ccccc3)cc2)cc1-c1cccc2ccccc12 ; *c1ccc(-c2ccc(-c3ccccc3)cc2)cc1-c1ccc2ccccc2c1 ; *c1ccc(-c2ccc(-c3ccccc3)cc2)cc1-c1ccccc1-c1ccccc1 ; *c1ccc(-c2ccccc2)cc1-c1ccccc1-c1ccccc1]

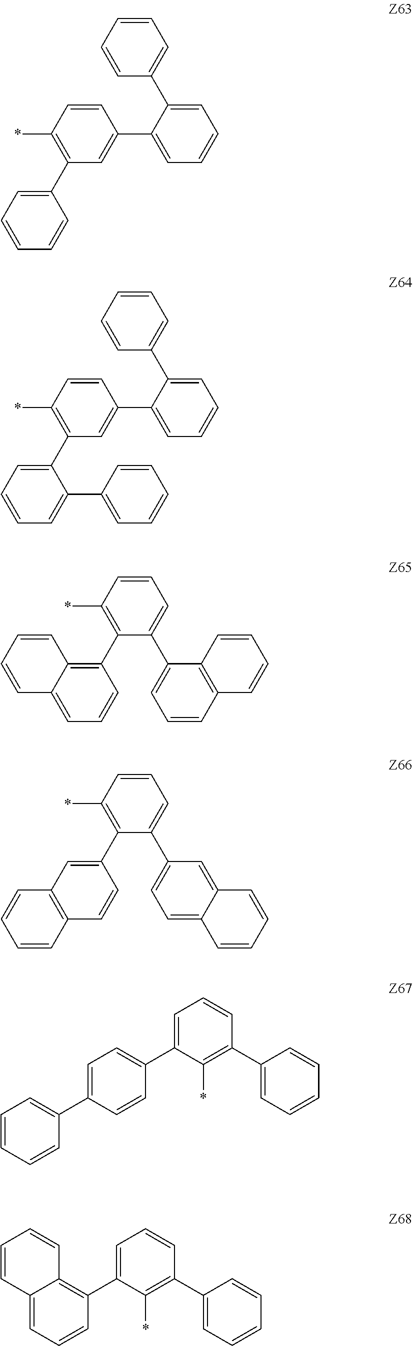 [SMILES: *c1ccc(-c2ccccc2-c2ccccc2)cc1-c1ccccc1 ; *c1ccc(-c2ccccc2-c2ccccc2)cc1-c1ccccc1-c1ccccc1 ; *c1cccc(-c2cccc3ccccc23)c1-c1cccc2ccccc12 ; *c1cccc(-c2ccc3ccccc3c2)c1-c1ccc2ccccc2c1 ; *c1c(-c2ccccc2)cccc1-c1ccc(-c2ccccc2)cc1 ; *c1c(-c2ccccc2)cccc1-c1cccc2ccccc12]

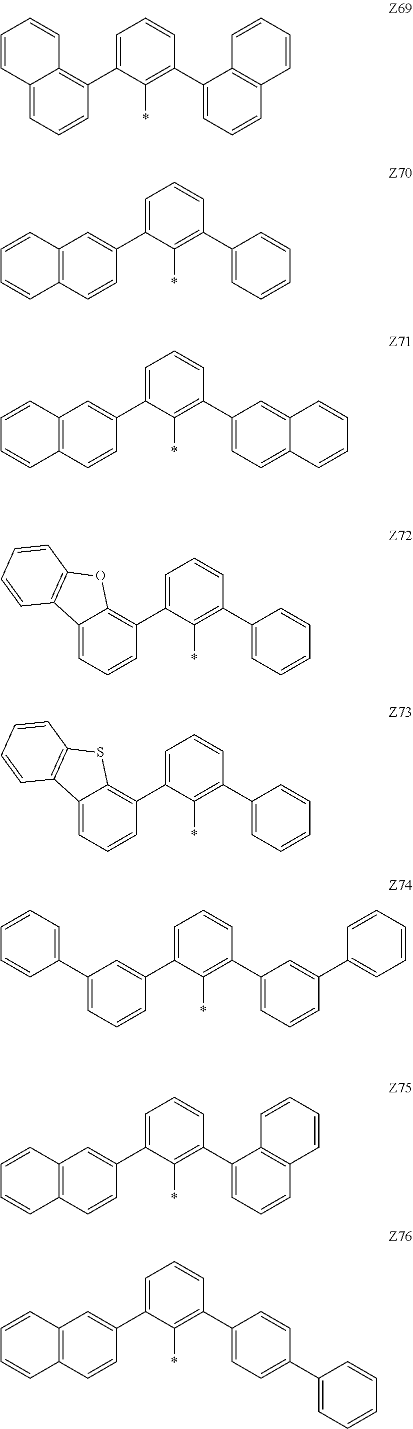

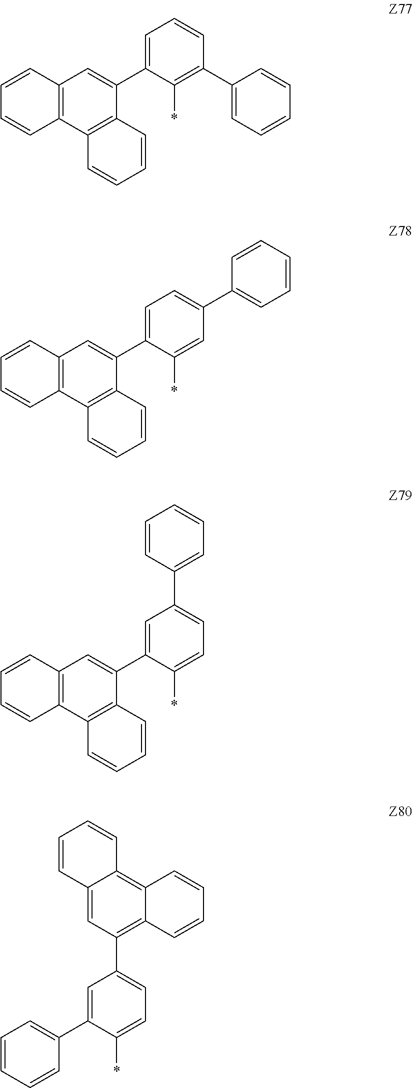 [SMILES: *c1c(-c2ccccc2)cccc1-c1cc2ccccc2c2ccccc12 ; *c1cc(-c2ccccc2)ccc1-c1cc2ccccc2c2ccccc12 ; *c1ccc(-c2ccccc2)cc1-c1cc2ccccc2c2ccccc12 ; *c1ccc(-c2cc3ccccc3c3ccccc23)cc1-c1ccccc1]

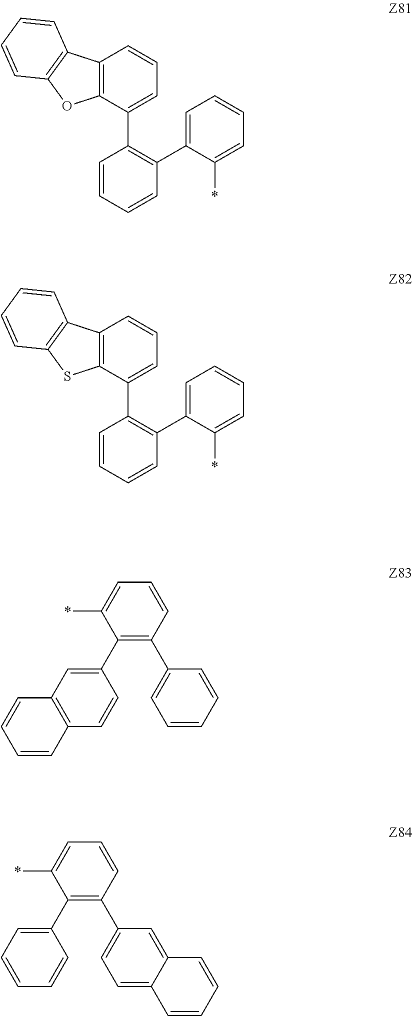 [SMILES: *c1ccccc1-c1ccccc1-c1cccc2c1oc1ccccc12 ; *c1ccccc1-c1ccccc1-c1cccc2c1sc1ccccc12 ; *c1cccc(-c2ccccc2)c1-c1ccc2ccccc2c1 ; *c1cccc(-c2ccc3ccccc3c2)c1-c1ccccc1]

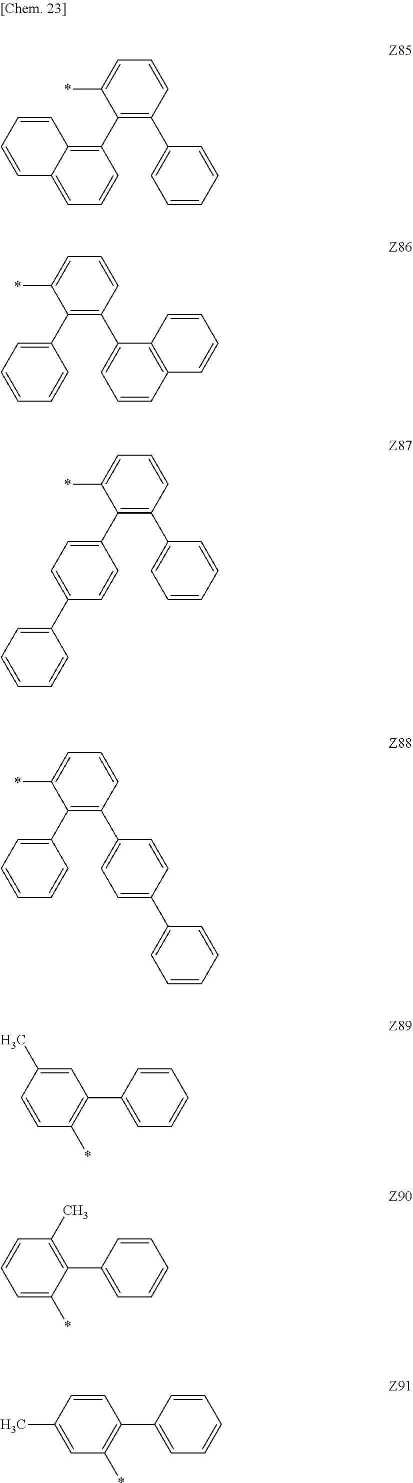

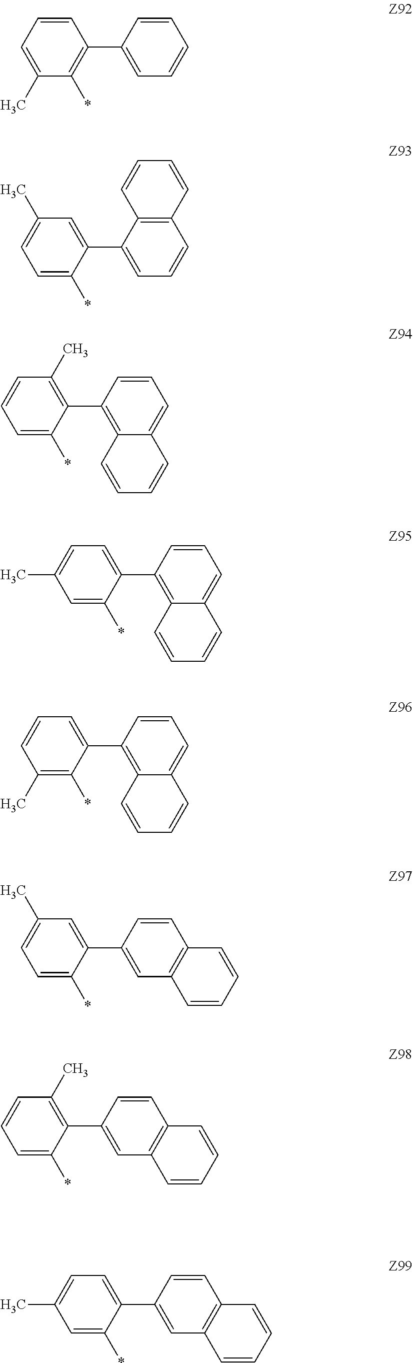

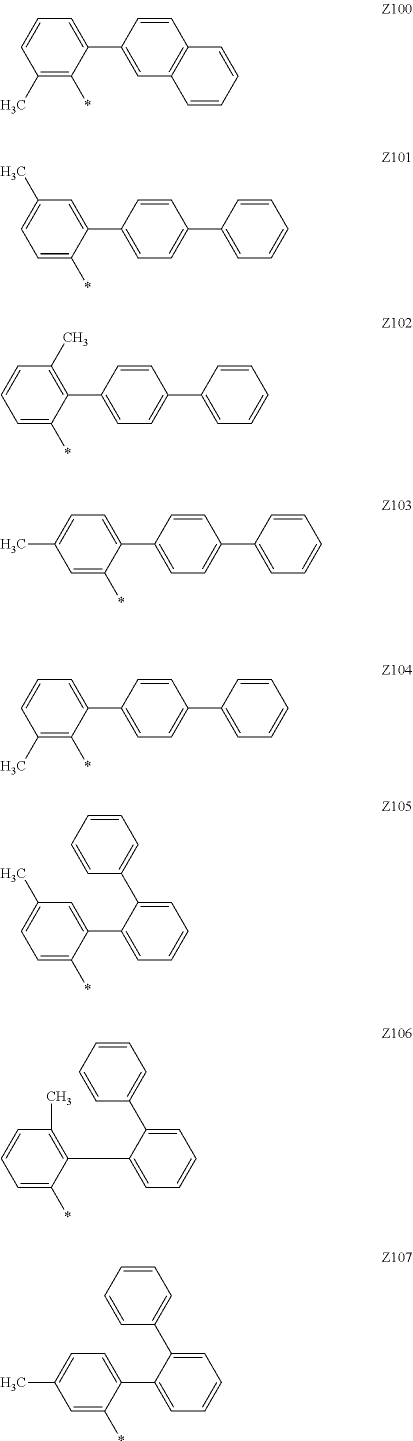

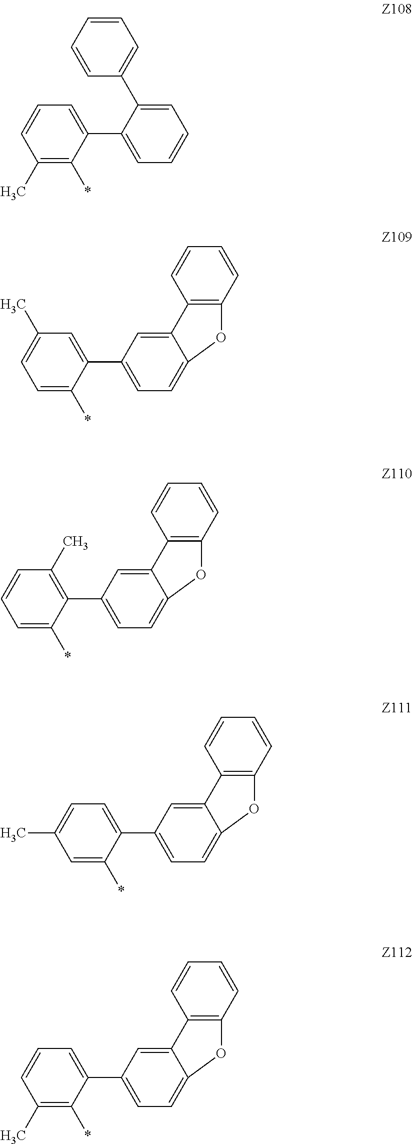 [SMILES: *c1c(C)cccc1-c1ccccc1-c1ccccc1 ; *c1ccc(C)cc1-c1ccc2oc3ccccc3c2c1 ; *c1cccc(C)c1-c1ccc2oc3ccccc3c2c1 ; *c1cc(C)ccc1-c1ccc2oc3ccccc3c2c1 ; *c1c(C)cccc1-c1ccc2oc3ccccc3c2c1]

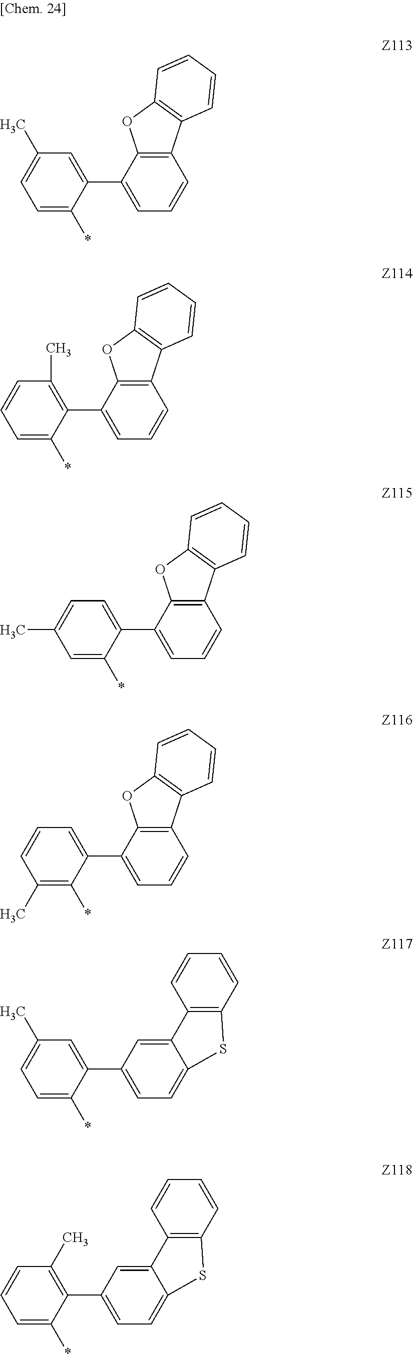 [SMILES: *c1ccc(C)cc1-c1cccc2c1oc1ccccc12 ; *c1cccc(C)c1-c1cccc2c1oc1ccccc12 ; *c1cc(C)ccc1-c1cccc2c1oc1ccccc12 ; *c1c(C)cccc1-c1cccc2c1oc1ccccc12 ; *c1ccc(C)cc1-c1ccc2sc3ccccc3c2c1 ; *c1cccc(C)c1-c1ccc2sc3ccccc3c2c1]

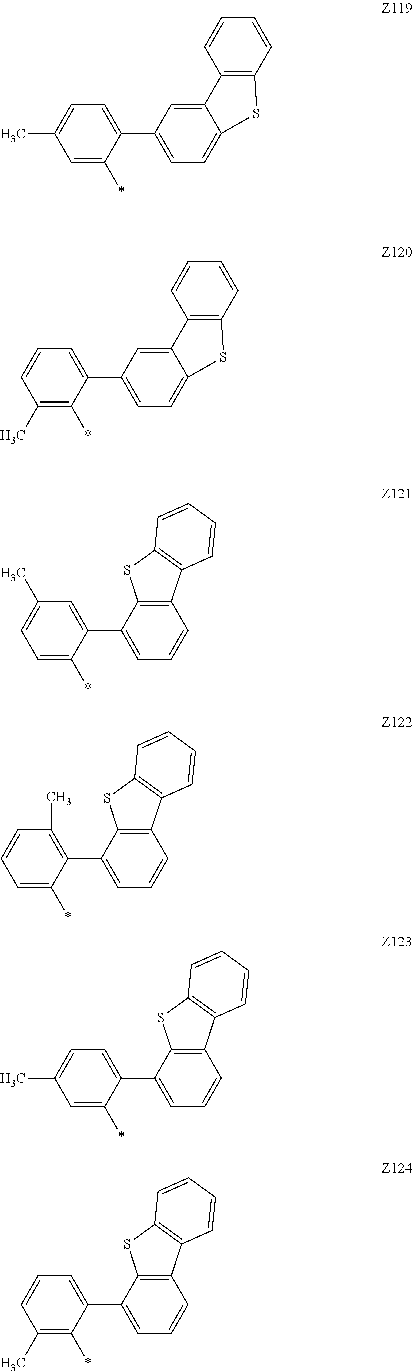 [SMILES: *c1cc(C)ccc1-c1ccc2sc3ccccc3c2c1 ; *c1c(C)cccc1-c1ccc2sc3ccccc3c2c1 ; *c1ccc(C)cc1-c1cccc2c1sc1ccccc12 ; *c1cccc(C)c1-c1cccc2c1sc1ccccc12 ; *c1cc(C)ccc1-c1cccc2c1sc1ccccc12 ; *c1c(C)cccc1-c1cccc2c1sc1ccccc12]

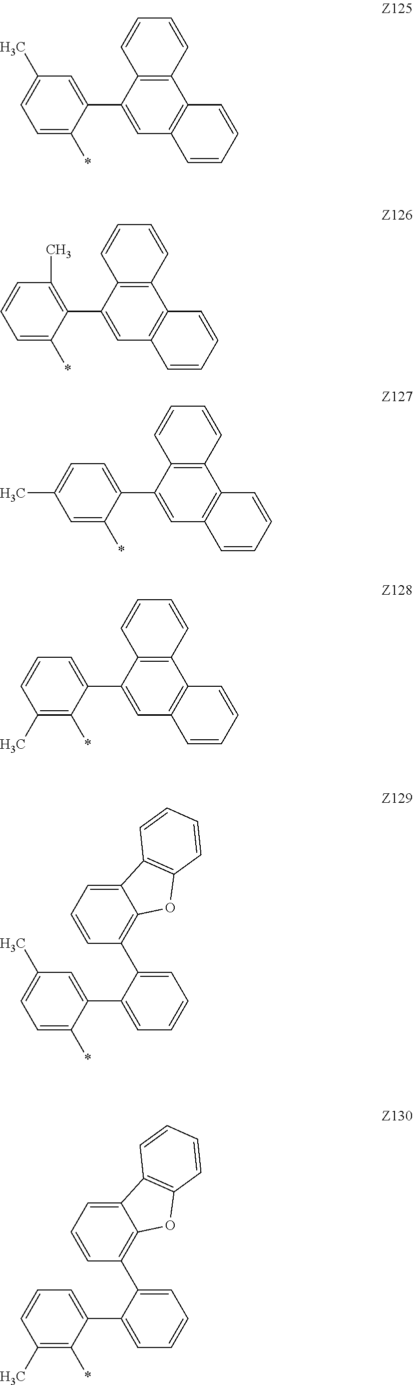

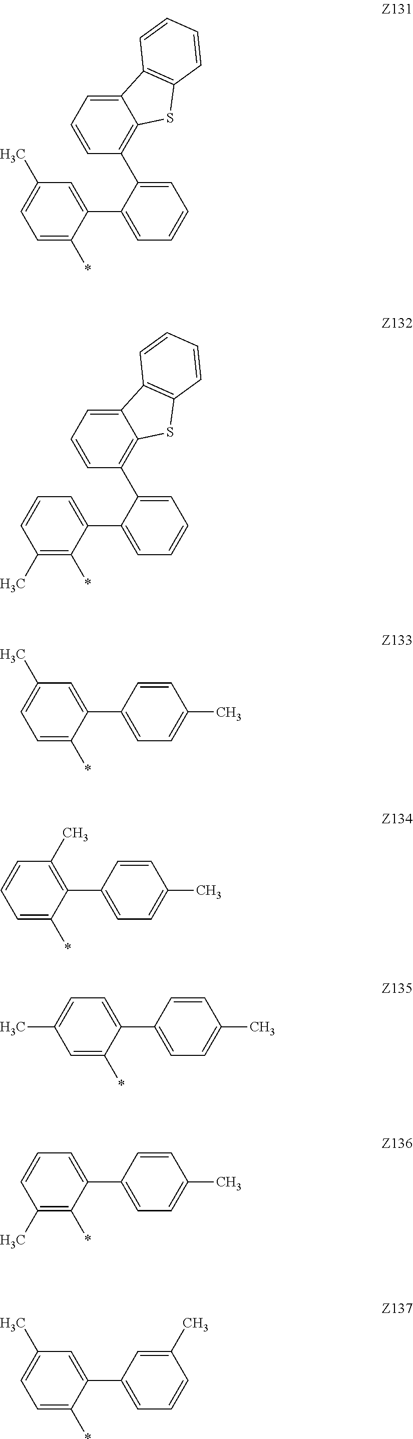

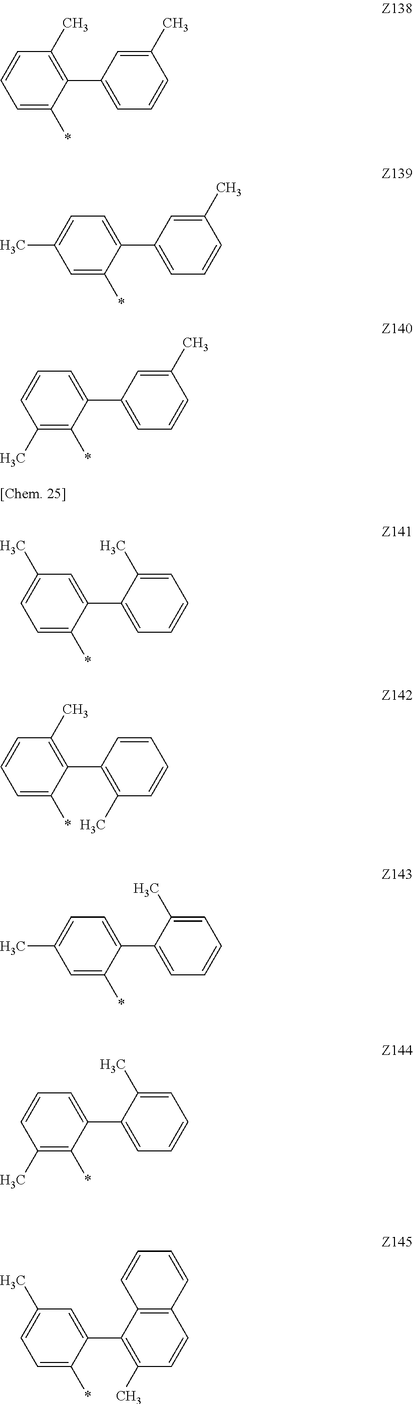

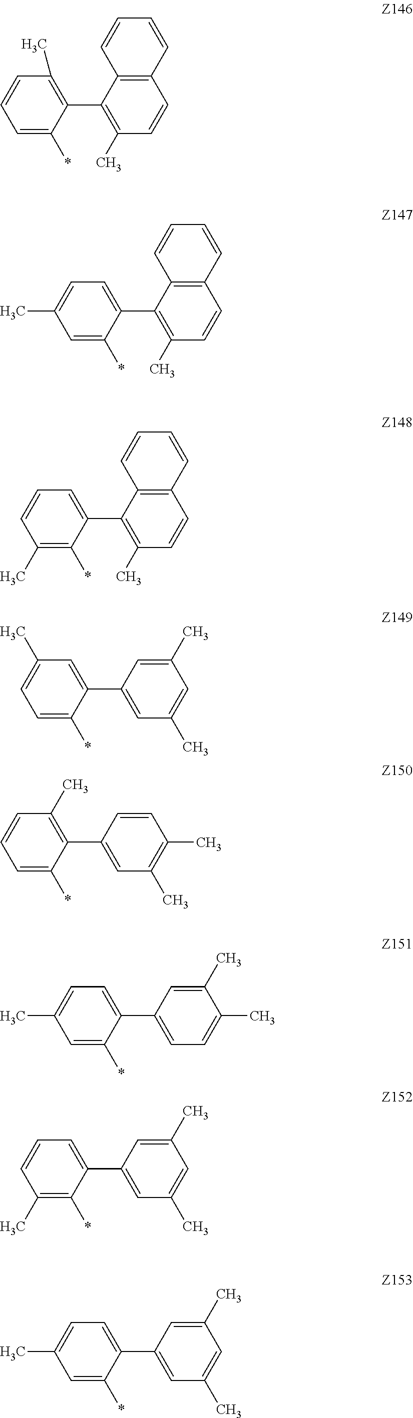

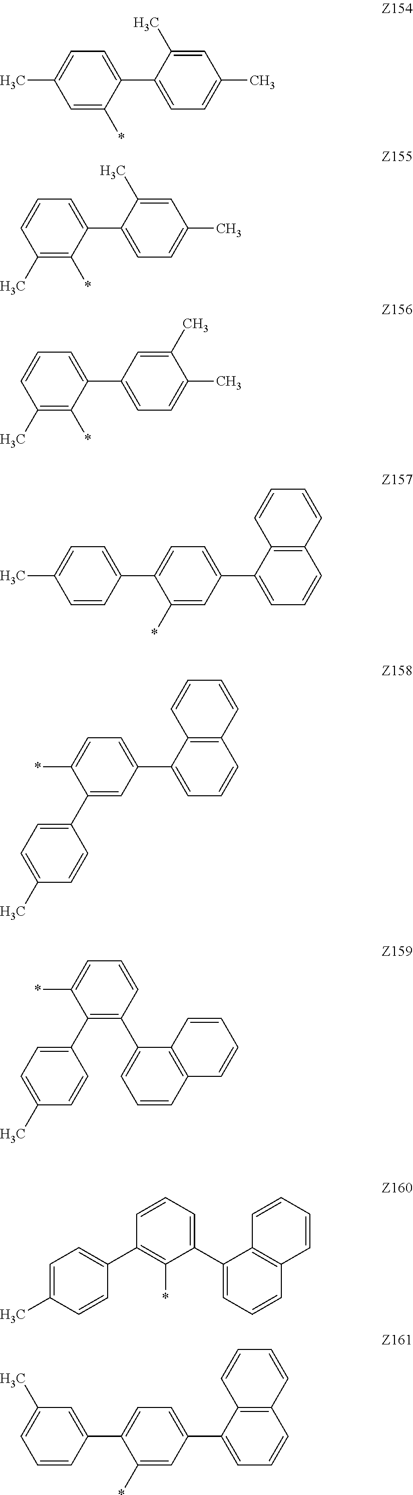

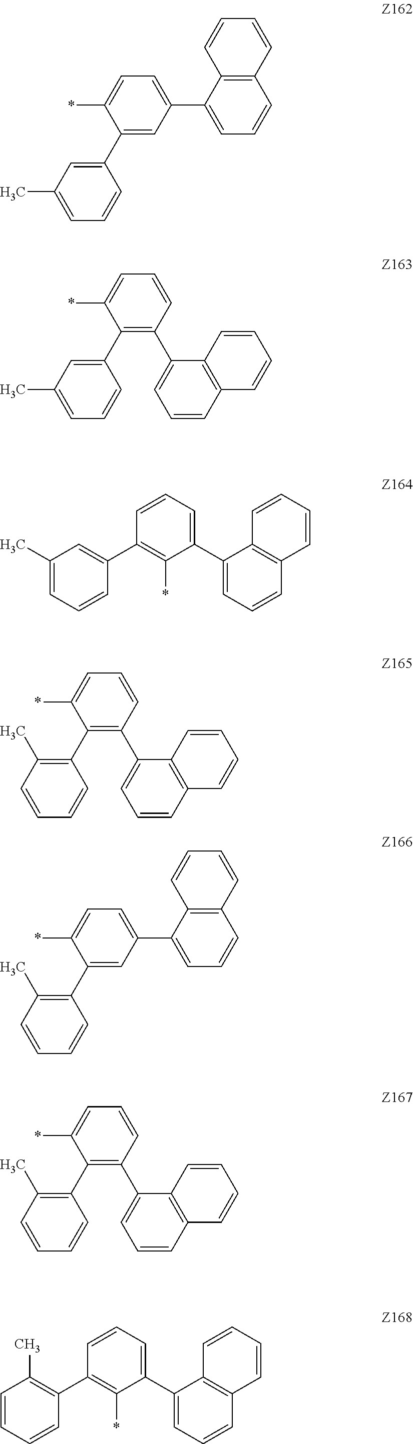

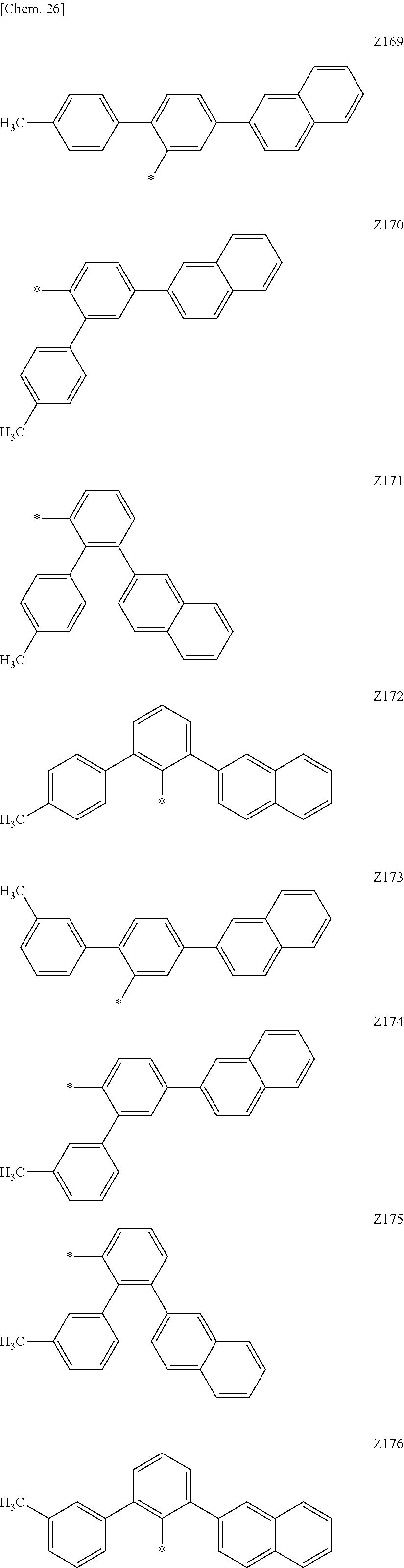

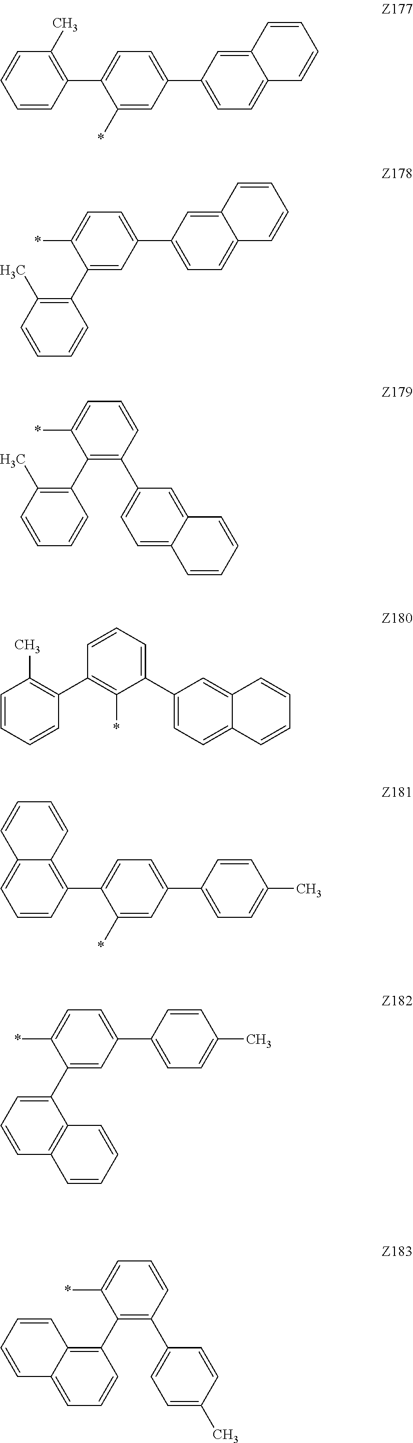

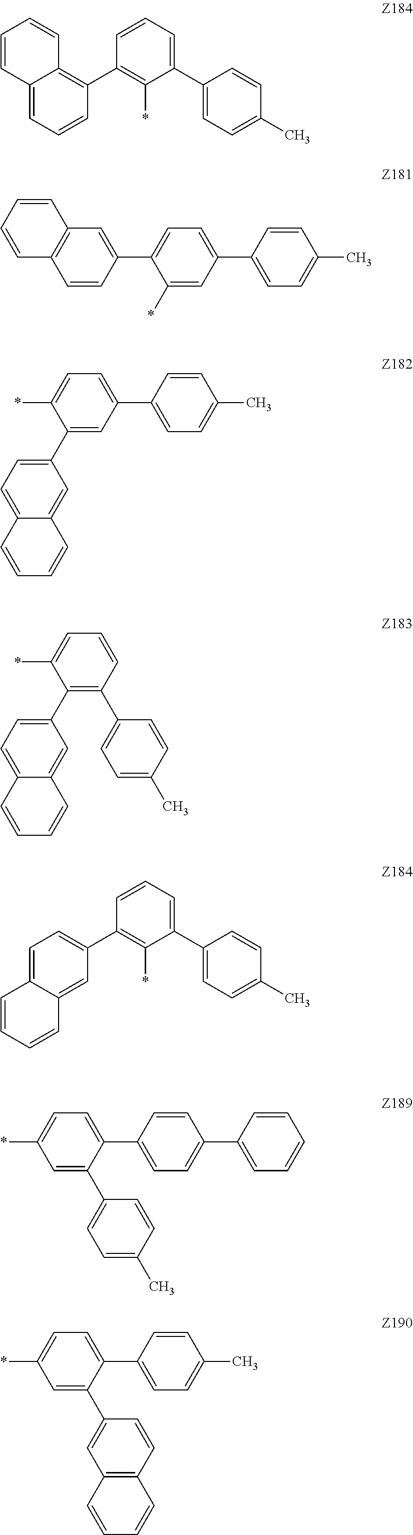

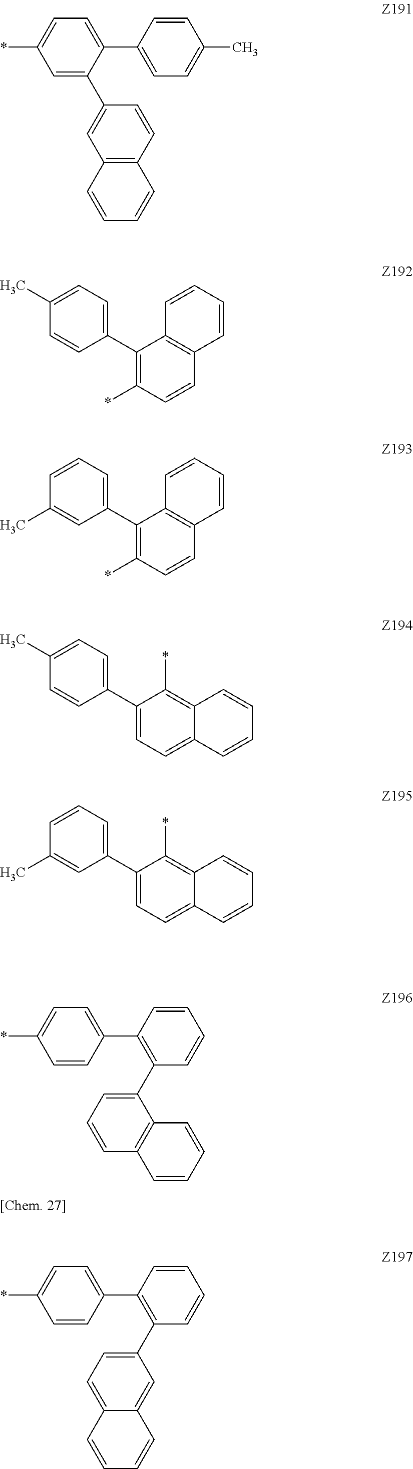

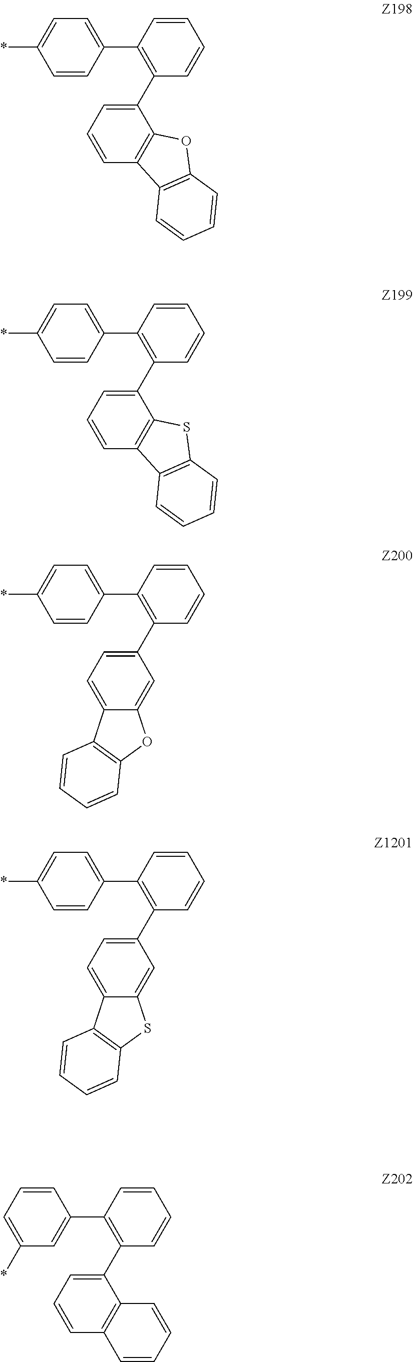

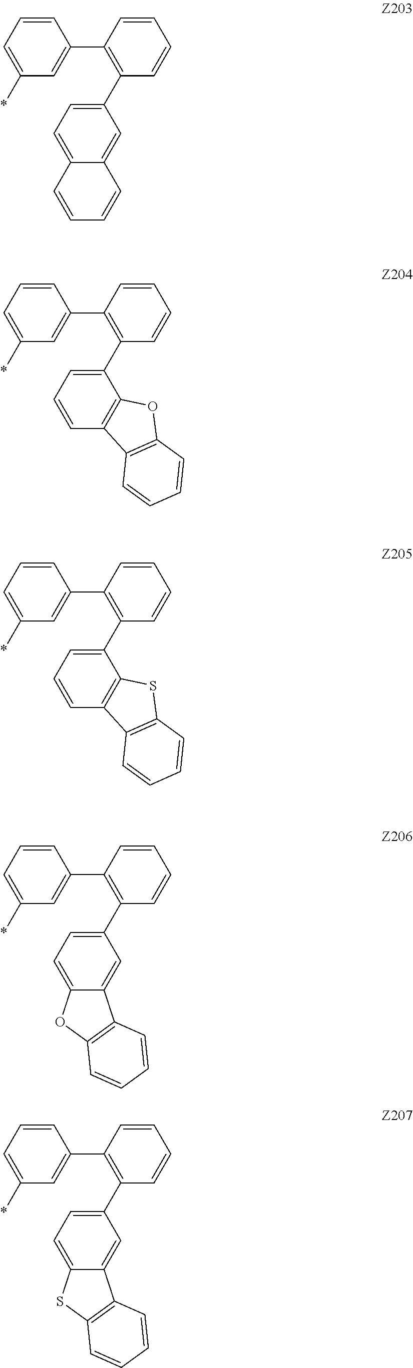

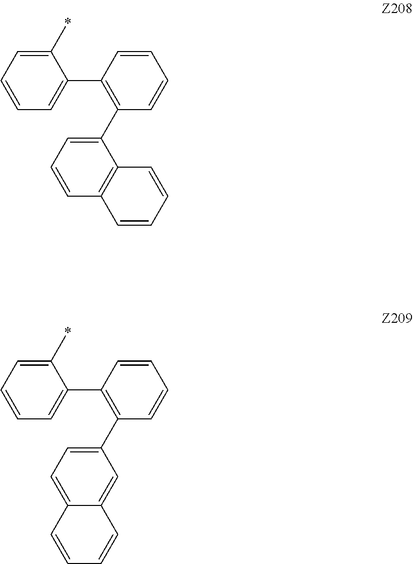 [SMILES: *c1ccccc1-c1ccccc1-c1cccc2ccccc12 ; *c1ccccc1-c1ccccc1-c1ccc2ccccc2c1]

[10] According to another aspect of the present disclosure,
there is provided a hole-injection layer comprising:
a first compound; and
a second compound,
wherein the first compound is
the transverse current suppressing material according to any one of [1] to [6] or
the carbazole compound according to [7] to [9], and
the second compound is an electron-accepting p-dopant.

[11] According to another aspect of the present disclosure,
there is provided the hole-injection layer according to [10], further comprising
a third compound,
wherein the third compound is a hole-transporting triarylamine compound.

[12] According to another aspect of the present disclosure,
there is provided the hole-injection layer according to [10] or [11], wherein the transverse current suppressing material according to any one of [1] to [6] or the carbazole compound according to [7] to [9] constitutes 20% by mass or more and 99.5% by mass or less.

[13] According to another aspect of the present disclosure,
there is provided an organic electroluminescent element comprising a hole-injection layer,
wherein the hole-injection layer contains the transverse current suppressing material according to any one of [1] to [6] or
the carbazole compound according to [7] to [9].

[14] According to another aspect of the present disclosure, there is provided the organic electroluminescent element according to [13], wherein the hole-injection layer is the hole-injection layer according to any one of [10] to [12].

[15] According to another aspect of the present disclosure,
there is provided the organic electroluminescent element according to [13], further comprising
a hole-transport layer,
wherein the hole-transport layer contains
the transverse current suppressing material according to any one of [1] to [6] or
the carbazole compound according to [7] to [9].

[16] According to another aspect of the present disclosure,
there is provided an organic electroluminescent element comprising:
a positive electrode;
a plurality of organic layers on the positive electrode; and
a negative electrode on the plurality of organic layers,
wherein one or more layers of the plurality of organic layers contain the carbazole compound according to [7] to [9].

### Advantageous Effects of Invention

According to one aspect of the present disclosure, it is possible to provide a transverse current suppressing material that suppresses a transverse current flowing in a horizontal direction with respect to a positive electrode film in an organic electroluminescent element, a carbazole compound, a hole-injection layer containing the transverse current suppressing material and the carbazole compound and an organic electroluminescent element that has good drive voltage, luminous efficiency and durability and has a lower transverse current.

### BRIEF DESCRIPTION OF DRAWINGS

FIG. 1 is a schematic cross-sectional view of an example of a layered configuration of an organic electroluminescent element according to an embodiment of the present disclosure.

### DESCRIPTION OF EMBODIMENTS

A transverse current suppressing material and a carbazole compound according to an embodiment of the present disclosure, a hole-injection layer containing the transverse current suppressing material and the carbazole compound and an organic electroluminescent element are described in detail below.

A transverse current refers to a current that flows unintentionally in a direction perpendicular to the stacking direction of an organic layer of an organic electroluminescent element, in other words, in a horizontal direction with respect to the main surface of a substrate. This transverse current causes a leakage current between a light-emitting pixel (a pixel intended to emit light) and an adjacent non-light-emitting pixel (a pixel not intended to emit light), causes an unintended pixel to emit light, and thereby impairs image quality. The transverse current is one of the causes of crosstalk in an organic electroluminescent element, and in recent years there has been a need for suppression of the generation of the transverse current due to an increase in demand for higher image quality.

### [Transverse Current Suppressing Material]

A transverse current suppressing material according to an embodiment of the present disclosure is represented by the formula (1):

[Chem. 28]

A-B Formula (1)

wherein
A is represented by the formula (2) or (3); and
B is represented by the formula (4);

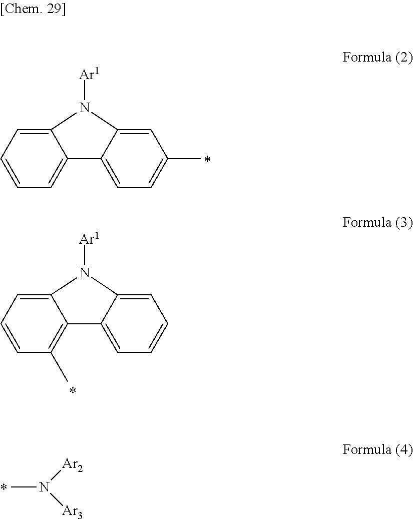

wherein
Ar¹ to Ar³ each independently denote
an optionally substituted monocyclic, linked or fused aromatic hydrocarbon group with 6 to 30 carbon atoms or
an optionally substituted monocyclic, linked or fused heteroaromatic group with 3 to 30 carbon atoms;
at least one of Ar¹ to Ar³ denotes a group represented by any one of the formulae (5) to (21);

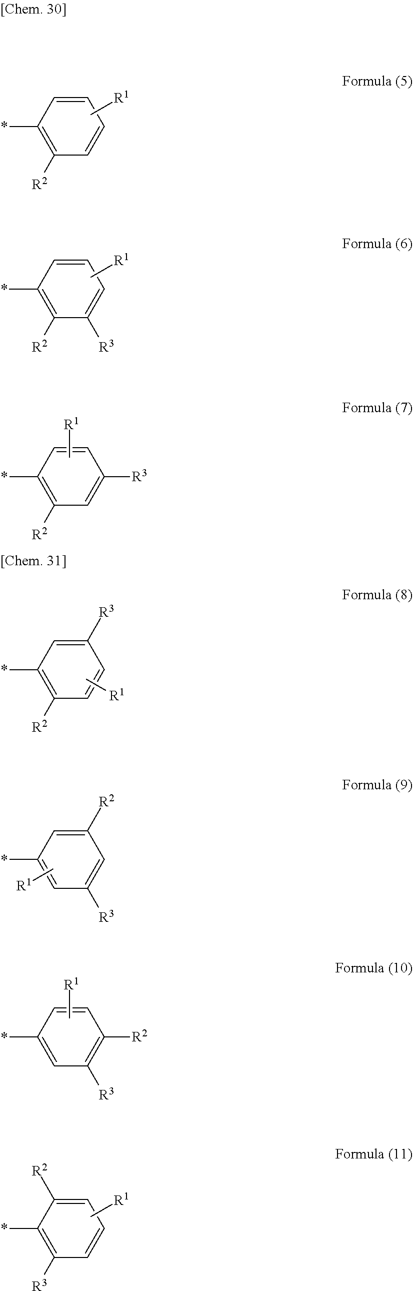

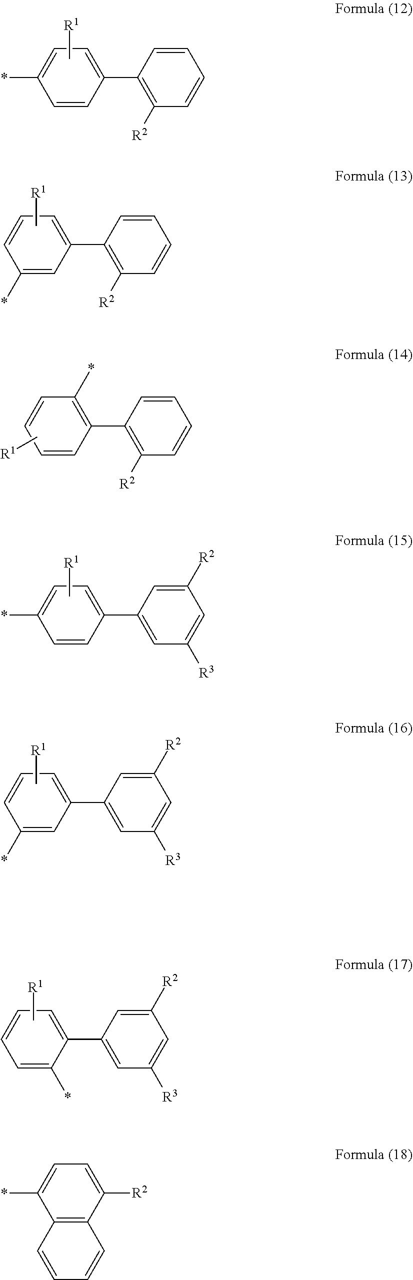

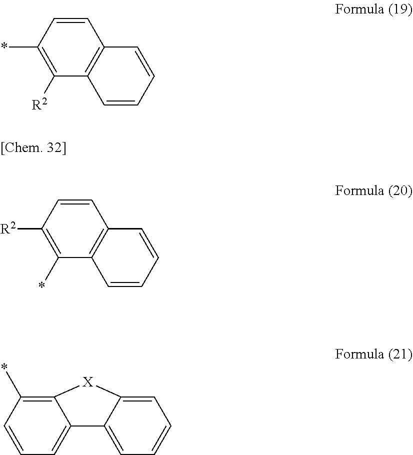

wherein
R¹ denotes a methyl group or a hydrogen atom;
R² and R³ each independently denote a phenyl group, a biphenylyl group, a naphthyl group, a phenanthryl group, a dibenzofuranyl group or a dibenzothienyl group each optionally substituted with a methyl group; and
X denotes an oxygen atom or a sulfur atom.

The monocyclic, linked or fused aromatic hydrocarbon group with 6 to 30 carbon atoms is, for example, a phenyl group, a biphenylyl group, a terphenylyl group, a naphthyl group, a fluorenyl group, a spirobifluorenyl group, a benzofluorenyl group, dibenzofluorenyl group, a phenanthryl group, a fluoranthenyl group, a triphenylenyl group, a pyrenyl group, an anthryl group, a tetracenyl group, a chrysenyl group, a perylenyl group, a pentacenyl group or one or more selected from the group consisting of benzene, naphthalene and phenanthrene fused to one of these groups.

The monocyclic, linked or fused heteroaromatic group with 3 to 30 carbon atoms is, for example, a pyrrolyl group, a thienyl group, a furyl group, an imidazolyl group, a thiazolyl group, an isothiazolyl group, an oxazolyl group, an isoxazolyl group, a pyridyl group, a pyrazyl group, an indolyl group, a benzothienyl group, a benzofuranyl group, a benzimidazolyl group, an indazolyl group, a benzothiazolyl group, a benzoisothiazolyl group, a 2,1,3-benzothiadiazolyl group, a benzoxazolyl group, a benzoisoxazolyl group, a 2,1,3-benzoxadiazolyl group, a quinolyl group, an isoquinolyl group, a carbazolyl group, a dibenzothienyl group, a dibenzofuranyl group, a phenoxazinyl group, a phenothiazinyl group, a phenazinyl group, a thianthrenyl group, or one or more selected from the group consisting of benzene, naphthalene and phenanthrene fused to one of these groups.

As described above, the monocyclic, linked or fused aromatic hydrocarbon group with 6 to 30 carbon atoms and the monocyclic, linked or fused heteroaromatic group with 3 to 30 carbon atoms may have a substituent. When these have a substituent, these are preferably each independently substituted with one or more groups selected from the group consisting of a linear, branched or cyclic alkyl group with 1 to 18 carbon atoms, a linear, branched or cyclic alkoxy group with 1 to 18 carbon atoms, an aromatic hydrocarbon group with 6 to 20 carbon atoms, a heteroaromatic group with 3 to 20 carbon atoms, a triphenylsilyl group, a cyano group, a fluorine atom and a deuterium atom. In this case, the number of substituents is not particularly limited.

The linear, branched or cyclic alkyl group with 1 to 18 carbon atoms is, for example, a methyl group, a propyl group, an isopropyl group, a butyl group, a sec-butyl group, a tert-butyl group, a pentyl group, a hexyl group, a heptyl group, an octyl group, a stearyl group, a cyclopropyl group, a cyclohexyl group or a trifluoromethyl group.

The linear, branched or cyclic alkoxy group with 1 to 18 carbon atoms is, for example, a propoxy group, an isopropoxy group, a n-butoxy group, a sec-butoxy group, a tert-butoxy group, a pentyloxy group, a hexyloxy group, a stearyloxy group, a difluoromethoxy group or a trifluoromethoxy group.

The aromatic hydrocarbon group with 6 to 20 carbon atoms is, for example, a phenyl group, a tolyl group, a biphenylyl group, a terphenylyl group, a naphthyl group, a fluorenyl group, a benzofluorenyl group, a dibenzofluorenyl group, a phenanthryl group, a triphenylenyl group, a pyrenyl group or an anthryl group.

The heteroaromatic group with 3 to 20 carbon atoms is, for example, a pyrrolyl group, a thienyl group, a furyl group, an imidazolyl group, a thiazolyl group, an isothiazolyl group, an oxazolyl group, an isoxazolyl group, a pyridyl group, a pyrazyl group, an indolyl group, a benzothienyl group, a benzofuranyl group, a benzimidazolyl group, an indazolyl group, a benzothiazolyl group, a benzoisothiazolyl group, a 2,1,3-benzothiadiazolyl group, a benzoxazolyl group, a benzoisoxazolyl group, a 2,1,3-benzoxadiazolyl group, a quinolyl group, an isoquinolyl group, a carbazolyl group, a dibenzothienyl group, a dibenzofuranyl group, a phenoxazinyl group, a phenothiazinyl group, a phenazinyl group or a thianthrenyl group.

Specific examples of Ar¹ to Ar³ include a phenyl group, a 4-methylphenyl group, a 3-methylphenyl group, a 2-methylphenyl group, a 2,4-dimethylphenyl group, a 2,5-dimethylphenyl group, a 3,4-dimethylphenyl group, a 3,5-dimethylphenyl group, a 2,6-dimethylphenyl group, a 2,3,5-trimethylphenyl group, a 2,3,6-trimethylphenyl group, a 3,4,5-trimethylphenyl group, a 4-biphenyl group, a 3-biphenyl group, a 2-biphenyl group, a 2-methyl-1,1′-biphenyl-4-yl group, a 3-methyl-1,1′-biphenyl-4-yl group, a 2′-methyl-1,1′-biphenyl-4-yl group, a 3′-methyl-1,1′-biphenyl-4-yl group, a 4′-methyl-1,1′-biphenyl-4-yl group, a 2,6-dimethyl-1,1′-biphenyl-4-yl group, a 2,2′-dimethyl-1,1′-biphenyl-4-yl group, a 2,3′-dimethyl-1,1′-biphenyl-4-yl group, a 2,4′-dimethyl-1,1′-biphenyl-4-yl group, a 3,2′-dimethyl-1,1′-biphenyl-4-yl group, a 2′,3′-dimethyl-1,1′-biphenyl-4-yl group, a 2′,4′-dimethyl-1,1′-biphenyl-4-yl group, a 2′,5′-dimethyl-1,1′-biphenyl-4-yl group, a 2′,6′-dimethyl-1,1′-biphenyl-4-yl group, a 3-methyl-1,1′-biphenyl-2-yl group, a 3′,5′-dimethyl-1,1′-biphenyl-4-yl group, a 4′-methyl-1,1′-biphenyl-2-yl group, a 3′-methyl-1,1′-biphenyl-2-yl group, a 2′-methyl-1,1′-biphenyl-2-yl group, a 3′,5′-dimethyl-1,1′-biphenyl-2-yl group, a 3′,4′-dimethyl-1,1′-biphenyl-2-yl group, a 3,3′,5′-trimethyl-1,1′-biphenyl-2-yl group, a 3,3′,4′-trimethyl-1,1′-biphenyl-2-yl group, a 3,4′-dimethyl-1,1′-biphenyl-2-yl group, a 3,3′-dimethyl-1,1′-biphenyl-2-yl group, a 3,2′-dimethyl-1,1′-biphenyl-2-yl group, a 3,3′,4′-trimethyl-1,1′-biphenyl-2-yl group, a 4,4′-dimethyl-1,1′-biphenyl-2-yl group, a p-terphenyl-2-yl group, p-terphenyl-3-yl group, a p-terphenyl-4-yl group, a p-terphenyl-2′-yl group, a m-terphenyl-2-yl group, a m-terphenyl-3-yl group, a m-terphenyl-4-yl group, a m-terphenyl-2′-yl group, a m-terphenyl-4′-yl group, a m-terphenyl-5′-yl group, a 3,3″-dimethyl-m-terphenyl-2′-yl group, a 4,4″-dimethyl-m-terphenyl-2′-yl group, a 3,5,3″,5″-tetramethyl-m-terphenyl-2′-yl group, a 3,4,3″,4″-tetramethyl-m-terphenyl-2′-yl group, an o-terphenyl-2-yl group, an o-terphenyl-3-yl group, an o-terphenyl-4-yl group, an o-terphenyl-3′-yl group, an o-terphenyl-4′-yl group, a 1-naphthyl group, a 2-naphthyl group, a 2-methylnaphthalen-1-yl group, 4-methylnaphthalen-1-yl group, a 6-methylnaphthalen-2-yl group, a 4-(1-naphthyl)phenyl group, a 4-(2-naphthyl)phenyl group, a 3-(1-naphthyl)phenyl group, 3-(2-naphthyl)phenyl group, a 3-methyl-4-(1-naphthyl)phenyl group, a 3-methyl-4-(2-naphthyl)phenyl group, a 3-methyl-2-(4-methyl-1-naphthyl)phenyl group, a 6-methyl-2-(4-methyl-1-naphthyl)phenyl group, a 2-(1-naphthyl)-6-methylphenyl group, a 2-(2-naphthyl)-6-methylphenyl group, 4-(1-naphthyl)biphenyl group, a 4-(2-naphthyl)biphenyl group, a 3-(1-naphthyl)biphenyl group, a 3-(2-naphthyl)biphenyl group, a 4-(2-methylnaphthalen-1-yl)phenyl group, a 3-(2-methylnaphthalen-1-yl)phenyl group, a 4-phenylnaphthalen-1-yl group, a 4-(2-methylphenyl)naphthalen-1-yl group, a 4-(3-methylphenyl)naphthalen-1-yl group, a 4-(4-methylphenyl)naphthalen-1-yl group, a 6-phenylnaphthalen-2-yl group, a 4-(2-methylphenyl)naphthalen-2-yl group, 4-(3-methylphenyl)naphthalen-2-yl group, a 4-(4-methylphenyl)naphthalen-2-yl group, a tetraphenylsilan-4-yl group, a tetraphenylsilan-3-yl group, a 2-fluorenyl group, a 9,9-dimethyl-2-fluorenyl group, a 9,9-diphenyl-2-fluorenyl group, a 9,9-diphenyl-4-fluorenyl group, a 9,9′-spirobifluoren-2-yl group, 9,9′-spirobifluoren-4-yl group, a 4-(9,9′-spirobifluoren-4-yl)phenyl group, a 3-(9,9′-spirobifluoren-4-yl) phenyl group, a 4-(9,9′-spirobifluoren-4-yl)biphenyl group, a 3-(9,9′-spirobifluoren-4-yl)biphenyl group, a 4-(9,9′-diphenyl fluoren-4-yl)phenyl group, a 3-(9,9′-diphenylfluoren-4-yl)phenyl group, a 4-(9,9′-diphenylfluoren-4-yl)biphenyl group, a 3-(9,9′-diphenylfluoren-4-yl)biphenyl group, a 3-(1-triphenylenyl)biphenyl group, a 9-phenanthryl group, a 2-phenanthryl group, a 4-(9-phenanthryl)phenyl group, a 3-(9-phenanthryl) Phenyl group, a 4-(9-phenanthryl)biphenyl group, a 3-(1-naphthyl)biphenyl group, a 3-(9-phenanthryl)biphenyl group, a 1-triphenylenyl group, a 2-triphenylenyl group, a 3-triphenylenyl group, a 4-triphenylenyl group, a 4-(1-triphenylenyl)phenyl group, a 3-(1-triphenylenyl)phenyl group, a 4-(1-triphenylenyl)biphenyl group, a 3-(1-triphenylenyl)biphenyl group, a 3-(1-triphenylenyl) biphenyl group, a 11,11′-dimethylbenzo[a]fluoren-9-yl group, a 11,11′-dimethylbenzo[a]fluoren-3-yl group, a 11,11′-dimethylbenzo[b]fluoren-9-yl group, a 11,11′-dimethylbenzo[b]fluoren-3-yl group, a 11,11′-dimethylbenzo[c]fluoren-9-yl group, a 11,11′-dimethylbenzo[c]fluoren-2-yl group, a 3-fluoranthenyl group, a 8-fluoranthenyl group, a 1-imidazolyl group, a 2-phenyl-1-imidazolyl group, a 2-phenyl-3,4-dimethyl-1-imidazolyl group, a 2, 3,4-triphenyl-1-imidazolyl group, a 2-(2-naphthyl)-3,4-dimethyl-1-imidazolyl group, a 2-(2-naphthyl)-3,4-diphenyl-1-imidazolyl group, a 1-methyl-2-imidazolyl group, a 1-ethyl-2-imidazolyl group, a 1-phenyl-2-imidazolyl group, a 1-methyl-4-phenyl-2-imidazolyl group, a 1-methyl-4,5-dimethyl-2-imidazolyl group, a 1-methyl-4,5-diphenyl-2-imidazolyl group, a 1-phenyl-4,5-dimethyl-2-imidazolyl group, a 1-phenyl-4,5-diphenyl-2-imidazolyl group, a 1-phenyl-4,5-dibiphenylyl-2-imidazolyl group, a 1-methyl-3-pyrazolyl group, a 1-phenyl-3-pyrazolyl group, a 1-methyl-4-pyrazolyl group, a 1-phenyl-4-pyrazolyl group, 1-methyl-5-pyrazolyl group, a 1-phenyl-5-pyrazolyl group, a 2-thiazolyl group, a 4-thiazolyl group, a 5-thiazolyl group, a 3-isothiazolyl group, a 4-isothiazolyl group, a 5-isothiazolyl group, a 2-oxazolyl group, a 4-oxazolyl group, a 5-oxazolyl group, a 3-isoxazolyl group, a 4-isoxazolyl group, a 5-isoxazolyl group, a 2-pyridyl group, a 3-methyl-2-pyridyl group, a 4-methyl-2-pyridyl group, a 5-methyl-2-pyridyl group, a 6-methyl-2-pyridyl group, a 3-pyridyl group, a 4-methyl-3-pyridyl group, a 4-pyridyl group, a 2-pyrimidyl group, a 2,2′-bipyridin-3-yl group, a 2,2′-bipyridin-4-yl group, a 2,2′-bipyridin-5-yl group, a 2,3′-bipyridin-3-yl group, a 2,3′-bipyridin-4-yl group, a 2,3′-bipyridin-5-yl group, a 5-pyrimidyl group, a pyrazyl group, a 1,3,5-triazyl group, a 4,6-diphenyl-1,3,5-triazin-2-yl group, a 1-benzimidazolyl group, a 2-methyl-1-benzimidazolyl group, a 2-phenyl-1-benzimidazolyl group, a 1-methyl-2-benzimidazolyl group, a 1-phenyl-2-benzimidazolyl group, a 1-methyl-5-benzimidazolyl group, a 1,2-dimethyl-5-benzimidazolyl group, a 1-methyl-2-phenyl-5-benzimidazolyl group, a 1-phenyl-5-benzimidazolyl group, a 1,2-diphenyl-5-benzimidazolyl group, a 1-methyl-6-benzimidazolyl group, a 1,2-dimethyl-6-benzimidazolyl group, a 1-methyl-2-phenyl-6-benzimidazolyl group, a 1-phenyl-6-benzimidazolyl group, a 1,2-diphenyl-6-benzimidazolyl group, a 1-methyl-3-indazolyl group, a 1-phenyl-3-indazolyl group, a 2-benzothiazolyl group, a 4-benzothiazolyl group, a 5-benzothiazolyl group, a 6-benzothiazolyl group, a 7-benzothiazolyl group, a 3-benzoisothiazolyl group, a 4-benzisothiazolyl group, a 5-benzisothiazolyl group, a 6-benzisothiazolyl group, a 7-benzisothiazolyl group, a 2,1,3-benzothiadiazol-4-yl group, a 2,1,3-benzothiadiazol-5-yl group, a 2-benzoxazolyl group, a 4-benzoxazolyl group, a 5-benzoxazolyl group, a 6-benzoxazolyl group, a 7-benzoxazolyl group, a 3-benzisoxazolyl group, a 4-benzisoxazolyl group, a 5-benzisoxazolyl group, a 6-benzisoxazolyl group, a 7-benzisoxazolyl group, a 2,1,3-Benzoxadiazolyl-4-yl group, a 2,1,3-benzoxadiazolyl-5-yl group, a 2-quinolyl group, a 3-quinolyl group, a 5-quinolyl group, a 6-quinolyl group, a 1-isoquinolyl group, a 4-isoquinolyl group, a 5-isoquinolyl group, a 2-acridinyl group, a 9-acridinyl group, a 1,10-phenanthrolin-3-yl group, a 1,10-phenanthrolin-5-yl group, a 2-thienyl group, a 3-thienyl group, a 2-benzothienyl group, a 3-benzothienyl group, a 2-dibenzothienyl group, a 4-dibenzothienyl group, a 2-furanyl group, a 3-furanyl group, a 2-benzofuranyl group, a 3-benzofuranyl group, a 2-dibenzofuranyl group, a 4-dibenzofuranyl group, a carbazol-9-yl group, a 9-methylcarbazol-2-yl group, a 9-methylcarbazol-3-yl group, a 9-methylcarbazol-4-yl group, a 9-phenylcarbazol-2-yl group, a 9-phenylcarbazol-3-yl group, a 9-phenylcarbazol-4-yl group, a 9-biphenylcarbazol-2-yl group, a 9-biphenylcarbazol-3-yl group, a 9-biphenylcarbazol-4-yl group, a 2-(9-carbazolyl)phenyl group, a 3-(9-carbazolyl)phenyl group, a 4-(9-carbazolyl)phenyl group, a 2-(9-carbazolyl)biphenyl group, a 3-(9-carbazolyl)biphenyl group, a 4-(9-carbazolyl)biphenyl group, a 2-(9-phenylcarbazol-3-yl)phenyl group, a 3-(9-phenylcarbazol-3-yl)phenyl group, a 4-(9-phenylcarbazol-3-yl)phenyl group, a 2-thianthryl group, a 10-phenylphenothiazin-3-yl group, a 10-phenylphenothiazin-2-yl group, a 10-phenylphenoxazin-3-yl group, a 10-phenylphenoxazin-2-yl group, a 1-methylindol-2-yl group, a 1-phenylindol-2-yl group, a 1-methylindol-2-yl group, a 1-phenylindol-2-yl group, a 4-(2-pyridyl)phenyl group, a 4-(3-pyridyl)phenyl group, a 4-(4-pyridyl)phenyl group, a 3-(2-pyridyl)phenyl group, a 3-(3-pyridyl)phenyl group, a 3-(4-pyridyl)phenyl group, a 4-(2-phenylimidazol-1-yl)phenyl group, a 4-(1-phenylimidazol-2-yl)phenyl group, a 4-(2,3,4-triphenylimidazol-1-yl)phenyl group, a 4-(1-methyl-4,5-diphenylimidazol-2-yl)phenyl group, a 4-(2-methylbenzimidazol-1-yl)phenyl group, a 4-(2-phenylbenzimidazol-1-yl) phenyl group, a 4-(1-methylbenzimidazol-2-yl)phenyl group, a 4-(2-phenylbenzimidazol-1-yl)phenyl group, a 3-(2-methylbenzimidazol-1-yl)phenyl group, 3-(2-phenylbenzimidazol-1-yl)phenyl group, a 3-(1-methylbenzimidazol-2-yl)phenyl group, a 3-(2-phenylbenzimidazol-1-yl)phenyl group, a 4-(3,5-diphenyltriazin-1-yl)phenyl group, a 4-(2-thienyl)phenyl group, a 4-(2-furanyl)phenyl group, a 5-phenylthiophen-2-yl group, a 5-phenylfuran-2-yl group, a 4-(5-phenylthiophen-2-yl)phenyl group, a 4-(5-phenylfuran-2-yl)phenyl group, a 3-(5-phenylthiophen-2-yl)phenyl group, 3-(5-phenylfuran-2-yl)phenyl group, a 4-(2-benzothienyl)phenyl group, a 4-(3-benzothienyl)phenyl group, a 3-(2-benzothienyl)phenyl group, a 3-(3-benzothienyl) phenyl group, a 4-(2-dibenzothienyl) phenyl group, a 4-(4-dibenzothienyl) phenyl group, a 3-(2-dibenzothienyl) phenyl group, a 3-(4-dibenzothienyl)) phenyl group, a 4-(2-dibenzofuranyl)phenyl group, a 4-(4-dibenzofuranyl)phenyl group, a 3-(2-dibenzofuranyl)phenyl group, a 3-(4-dibenzofuranyl)phenyl group, a 4-(2-benzothienyl)phenyl group, a 4-(3-benzothienyl)phenyl group, a 3-(2-benzothienyl)biphenyl group, a 3-(3-benzothienyl)biphenyl group, a 4-(2-Dibenzothienyl)biphenyl group, a 4-(4-dibenzothienyl)biphenyl group, a 3-(2-dibenzothienyl)biphenyl group, a 3-(4-dibenzothienyl)biphenyl group, a 4-(2-dibenzofuranyl)biphenyl group, a 4-(4-dibenzofuranyl)biphenyl group, a 3-(2-dibenzofuranyl)biphenyl group, a 3-(4-dibenzofuranyl)biphenyl group, a 5-phenylpyridin-2-yl group, a 4-phenylpyridin-2-yl group and a 5-phenylpyridin-3-yl group.

Due to good hole-transport properties, the optionally substituted monocyclic, linked or fused aromatic hydrocarbon group with 6 to 30 carbon atoms or the optionally substituted monocyclic, linked or fused heteroaromatic group with 3 to 30 carbon atoms in Ar¹ to Ar³ of the formula (1) preferably each independently denotes
(i) a phenyl group, a biphenylyl group, a terphenylyl group, a naphthyl group, a fluorenyl group, a spirobifluorenyl group, a benzofluorenyl group, a phenanthryl group, a fluoranthenyl group, a triphenylenyl group, an anthryl group, a pyrenyl group, a benzofuranyl group, a benzothienyl group, a dibenzofuranyl group or a dibenzothienyl group,
(ii) the group represented by (i) substituted with one or more groups selected from the group consisting of a methyl group, an ethyl group, a methoxy group, an ethoxy group, a cyano group, a deuterium atom, a fluorine atom, a phenyl group, a biphenylyl group, a naphthyl group, a phenanthryl group, a triphenylsilyl group, a carbazolyl group, a dibenzothienyl group and a dibenzofuranyl group or
(iii) a group represented by any one of the formulae (5) to (21).

Due to good hole-transport properties, the optionally substituted monocyclic, linked or fused aromatic hydrocarbon group with 6 to 30 carbon atoms or the optionally substituted monocyclic, linked or fused heteroaromatic group with 3 to 30 carbon atoms in Ar¹ to Ar³ more preferably each independently denotes
(i′) a phenyl group, a biphenylyl group, a terphenylyl group, a naphthyl group, a fluorenyl group, a spirobifluorenyl group, a benzofluorenyl group, a phenanthryl group, a fluoranthenyl group, a triphenylenyl group, an anthryl group, a pyrenyl group, a dibenzofuranyl group or a dibenzothienyl group or
(ii′) the group represented by (i′) substituted with one or more groups selected from the group consisting of a methyl group, a phenyl group, a biphenylyl group, a naphthyl group, a phenanthryl group, a triphenylsilyl group, a carbazolyl group, a dibenzothienyl group and a dibenzofuranyl group.

Due to good hole-transport properties, Ar¹ to Ar³ still more preferably each independently denote a phenyl group, a methylphenyl group, a biphenylyl group, a methylbiphenylyl group, a dimethylbiphenylyl group, a trimethylterphenylyl group, a terphenylyl group, a methylterphenylyl group, a dimethylterphenylyl group, a naphthyl group, a 9,9-dimethylfluorenyl group, a 9,9-diphenylfluorenyl group, a spirobifluorenyl group, 11,11-dimethylbenzo[a]fluorene, 11,11-dimethylbenzo[b]fluorene, 7,7-dimethylbenzo[c]fluorene, a phenanthryl group, a fluoranthenyl group, a triphenylenyl group, a naphthyl phenyl group, a phenanthryl phenyl group, a triphenylsilyl phenyl group, a carbazolyl phenyl group, a dibenzofuranyl group, a dibenzothienyl group, a dibenzofuranyl phenyl group or a dibenzothienyl phenyl group.

To suppress a transverse current, Ar¹ preferably denotes a group represented by any one of the formulae (5) to (21).

To suppress a transverse current, both Ar¹ and Ar² more preferably each independently denote a group represented by any one of the formulae (5) to (21).

To suppress a transverse current, both Ar¹ and Ar² still more preferably denote a group represented by any one of the formulae (6) to (8), (10) to (14) and (18) to (20).

In the formulae (5) to (21), R² and R³ preferably each independently denote a phenyl group, a methylphenyl group, a dimethylphenyl group, a trimethylphenyl group, a biphenylyl group, a methylbiphenylyl group, a dimethylbiphenylyl group, a naphthyl group, a phenanthryl group, a dibenzofuranyl group or a dibenzothienyl group.

To suppress a transverse current, the formulae (5) to (21) are more preferably groups represented by any one of the following formulae (Y1) to (Y298).

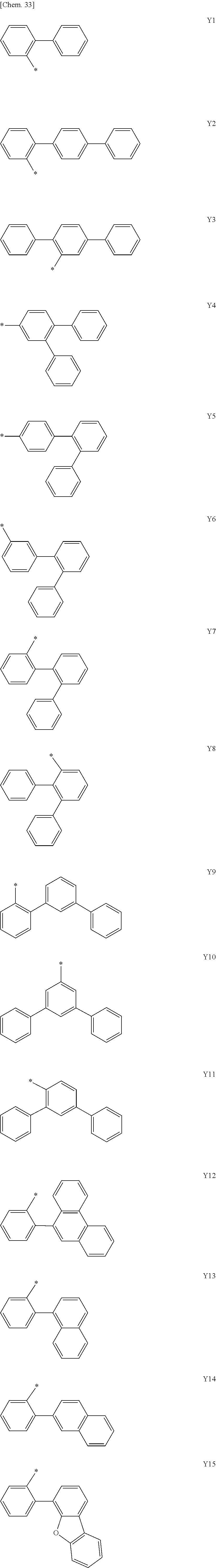

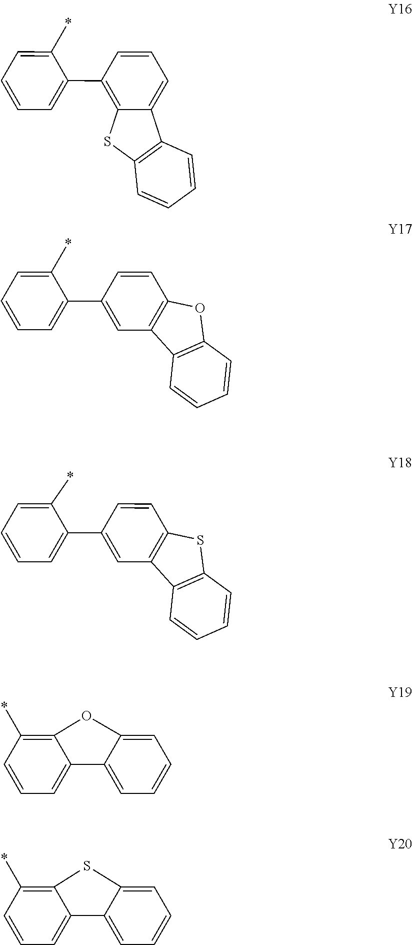 [SMILES: *c1ccccc1-c1cccc2c1sc1ccccc12 ; *c1ccccc1-c1ccc2oc3ccccc3c2c1 ; *c1ccccc1-c1ccc2sc3ccccc3c2c1 ; *c1cccc2c1oc1ccccc12 ; *c1cccc2c1sc1ccccc12]

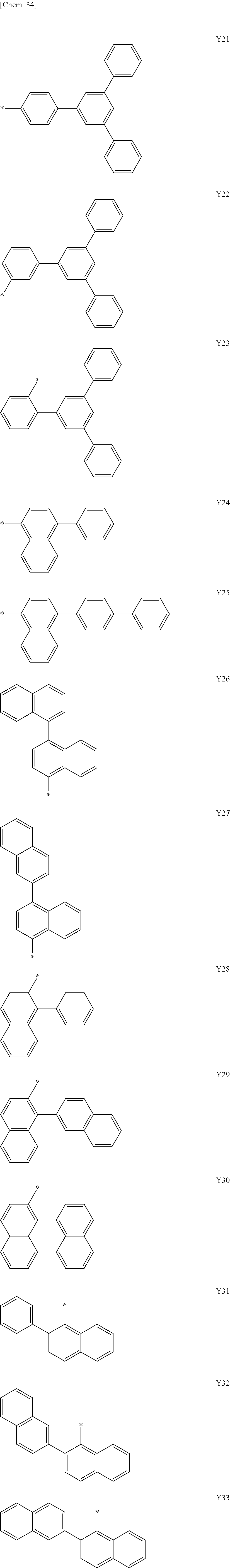

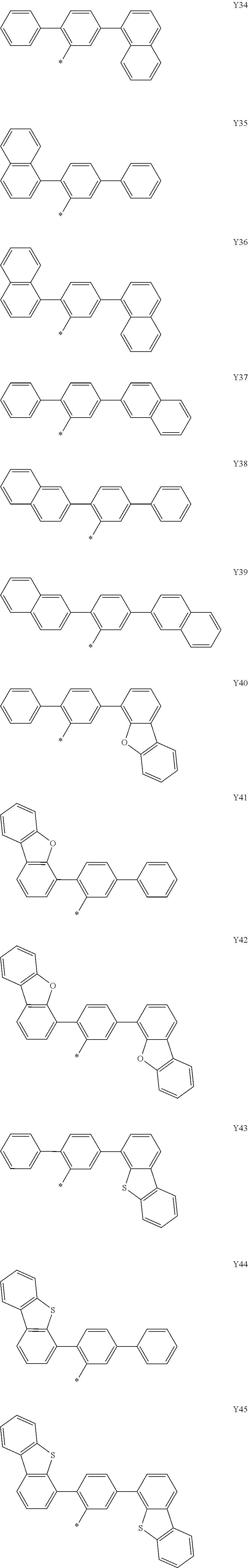

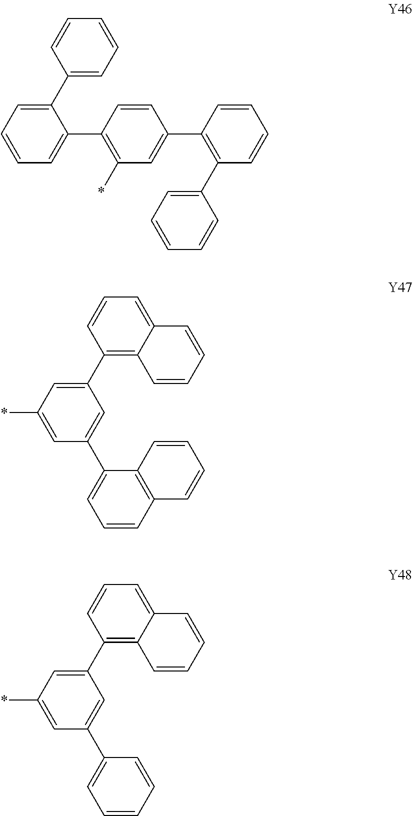 [SMILES: *c1cc(-c2ccccc2-c2ccccc2)ccc1-c1ccccc1-c1ccccc1 ; *c1cc(-c2cccc3ccccc23)cc(-c2cccc3ccccc23)c1 ; *c1cc(-c2ccccc2)cc(-c2cccc3ccccc23)c1]

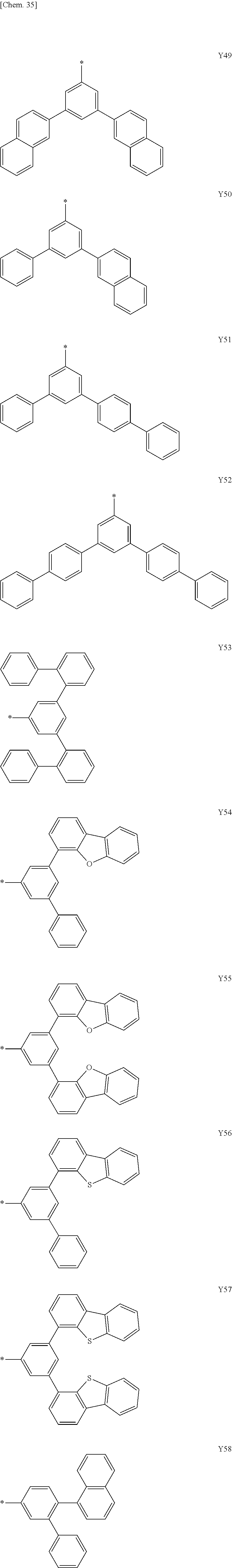

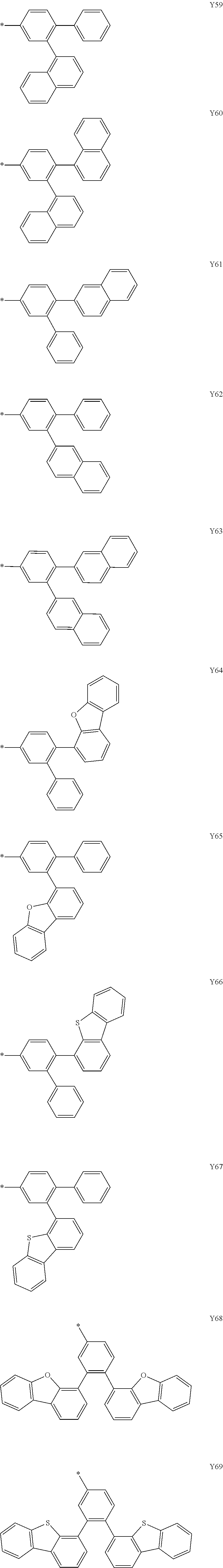

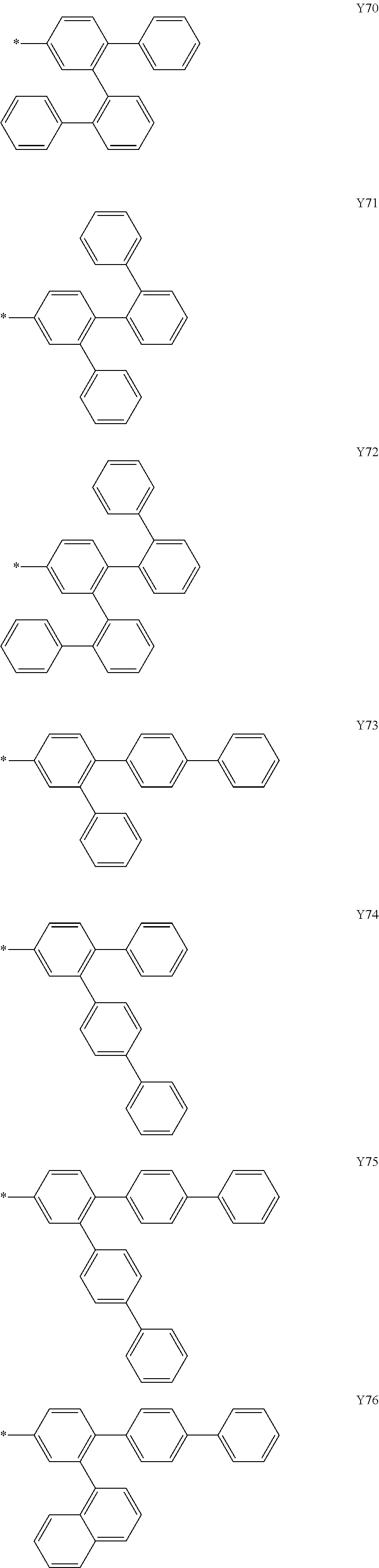

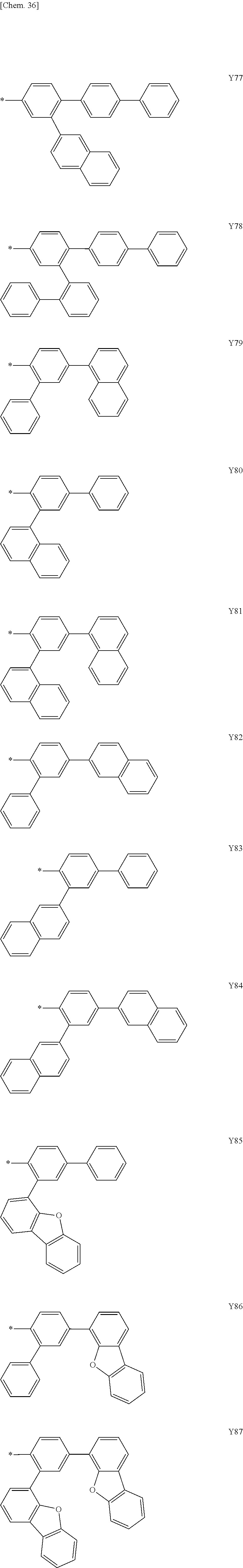

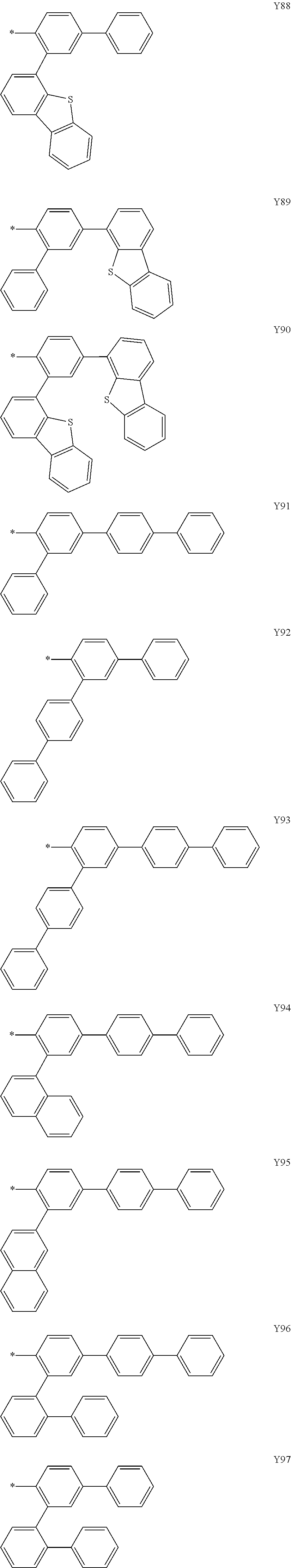

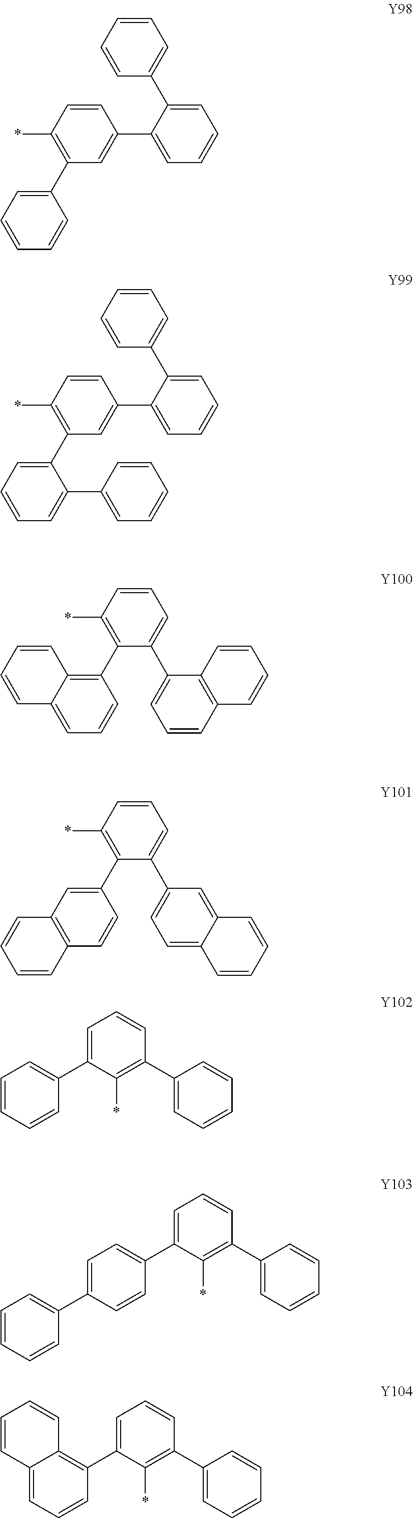

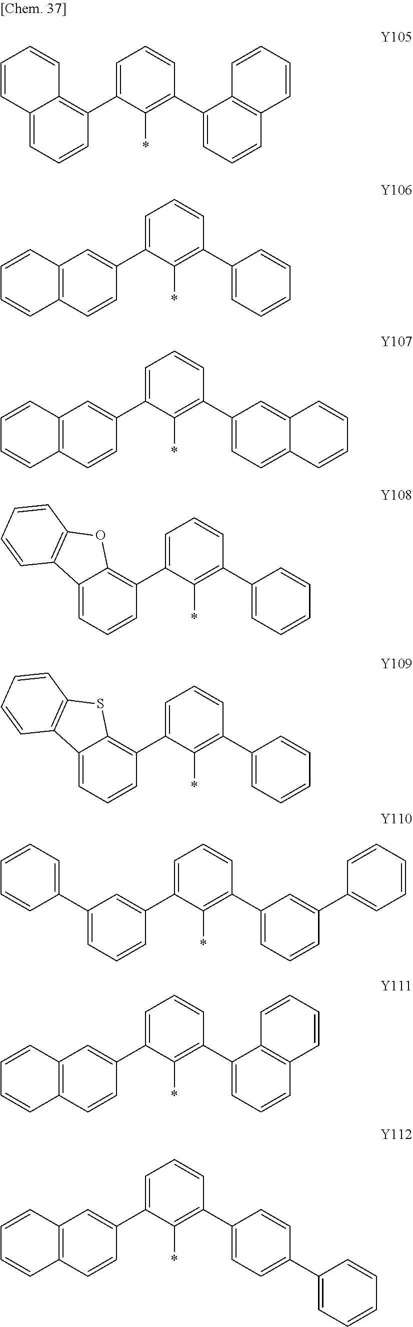

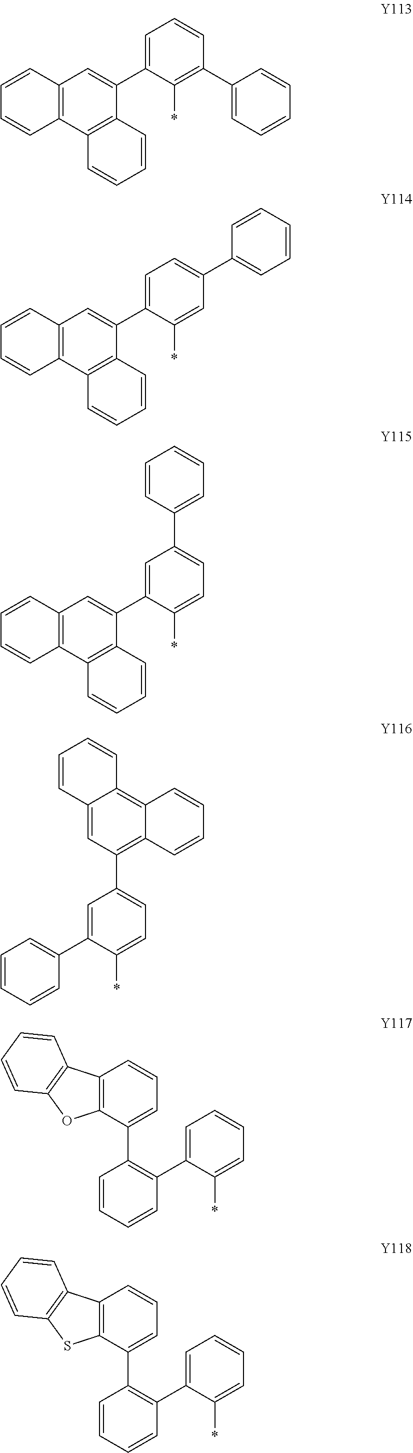

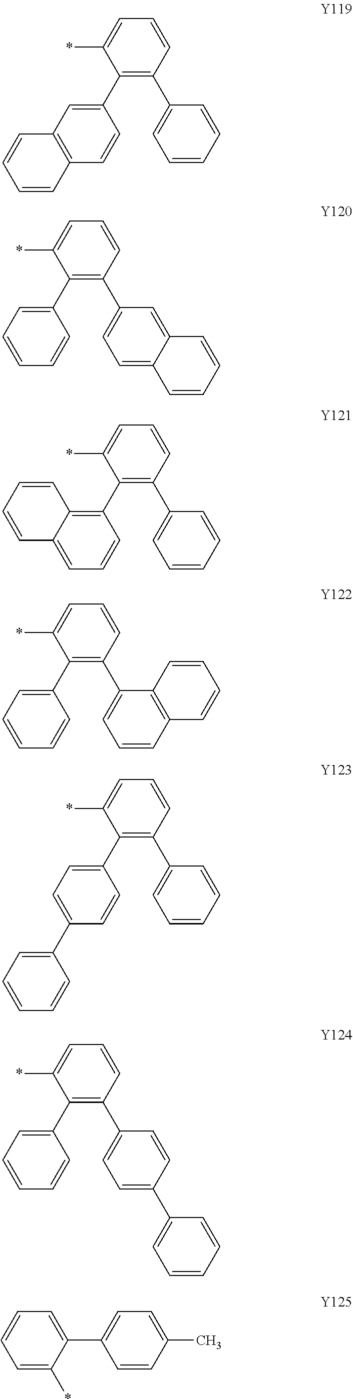

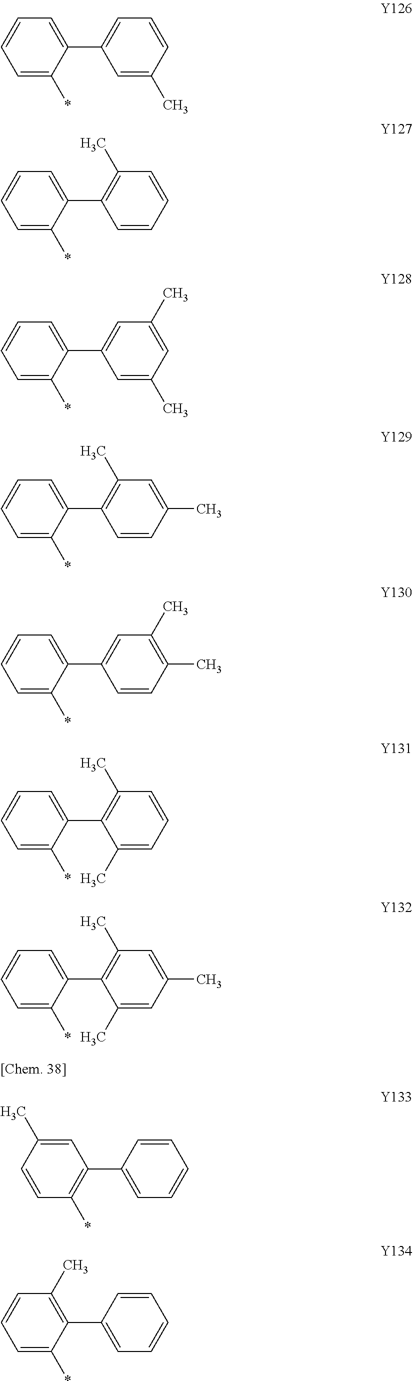

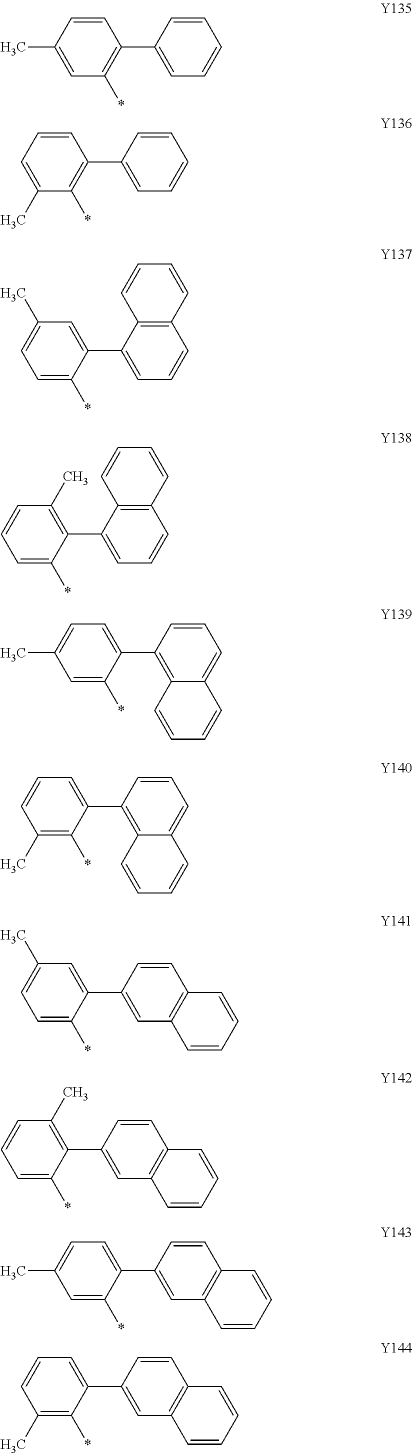

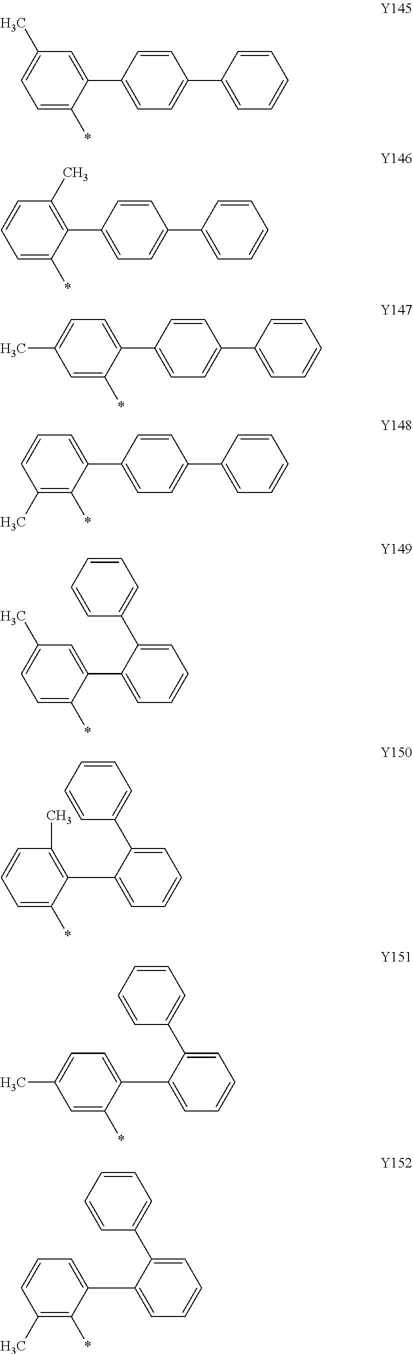

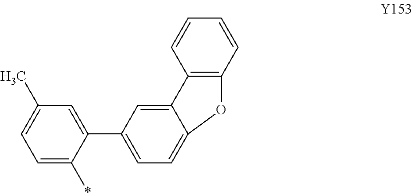 [SMILES: *c1ccc(C)cc1-c1ccc2oc3ccccc3c2c1]

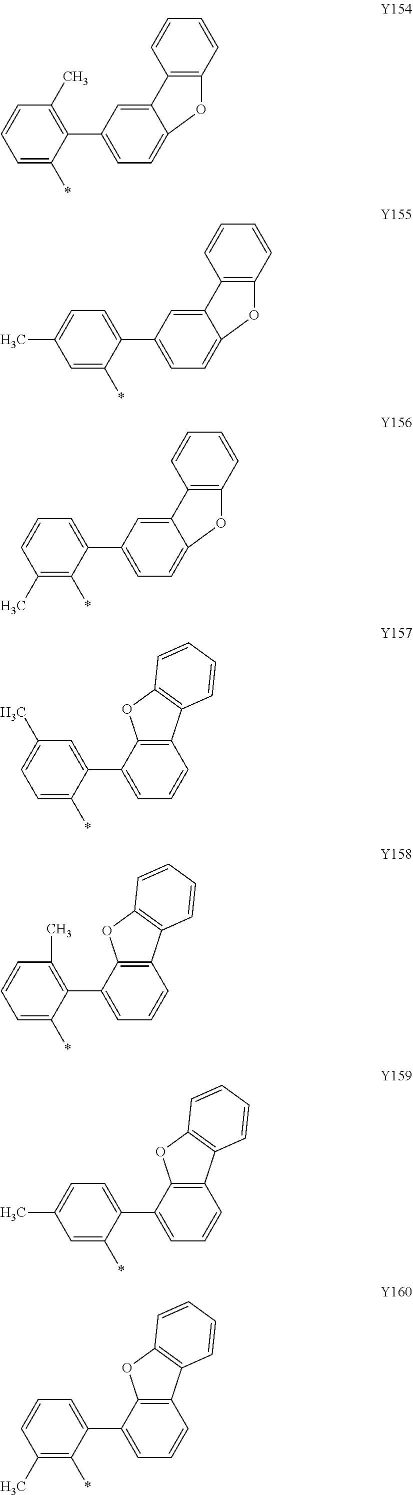

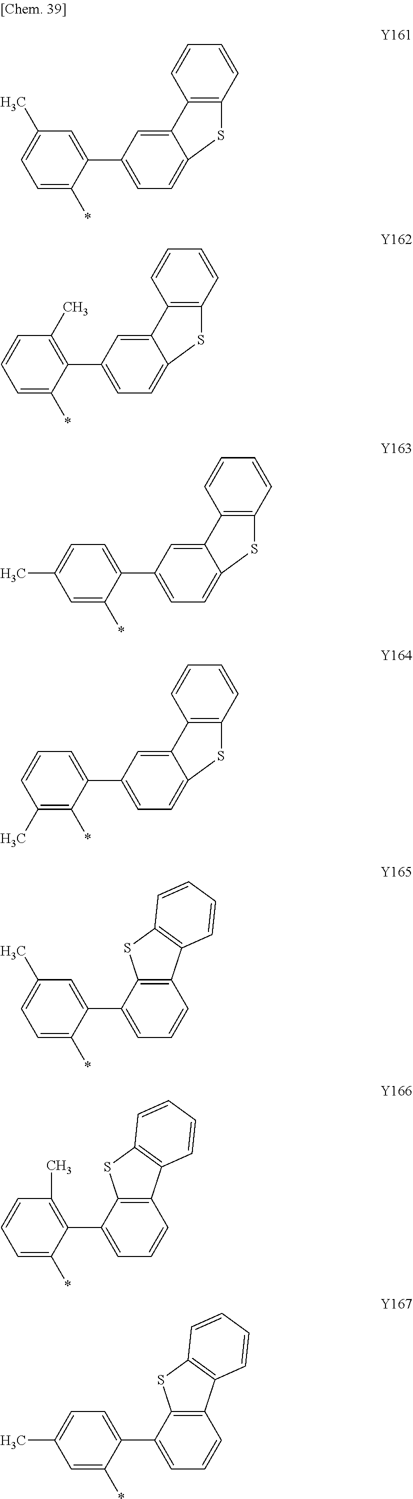

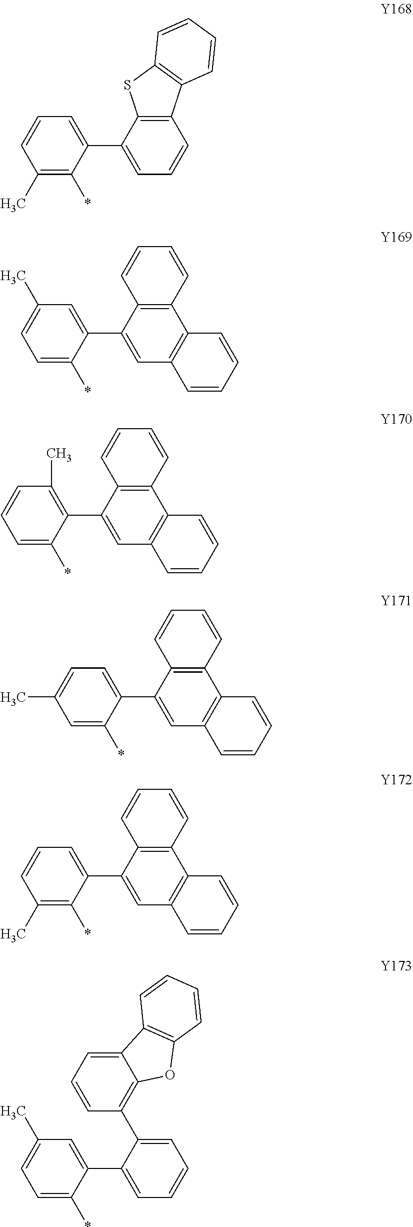

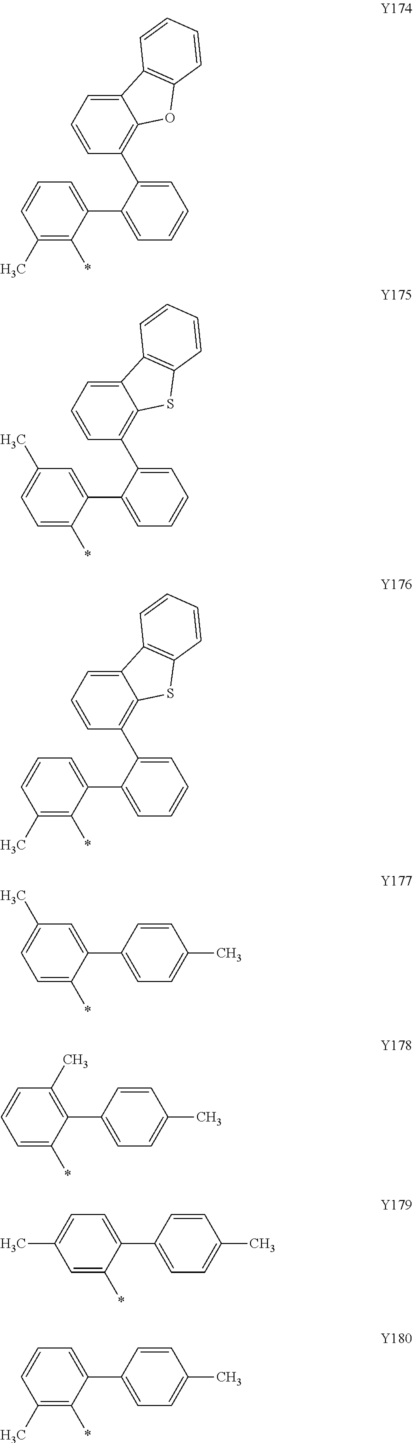

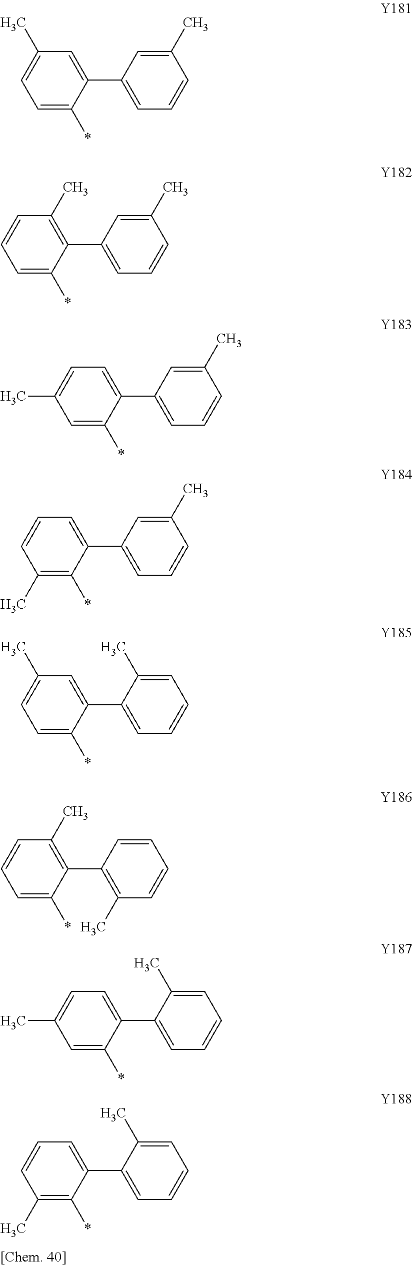

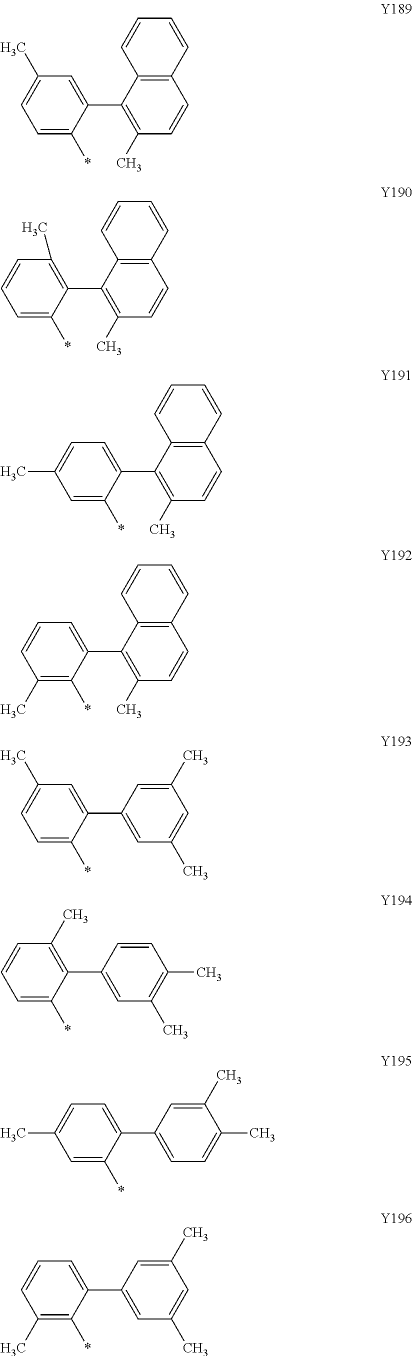

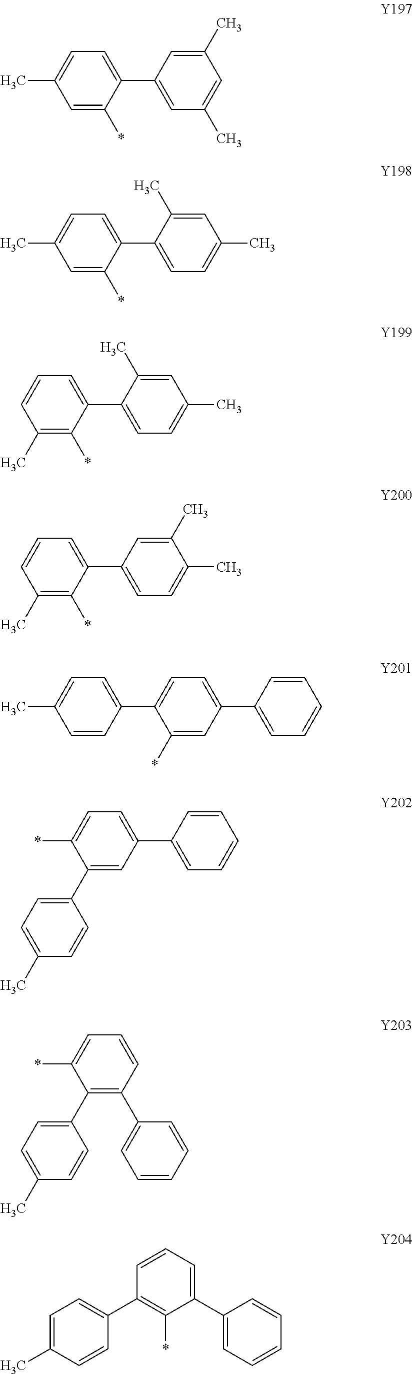

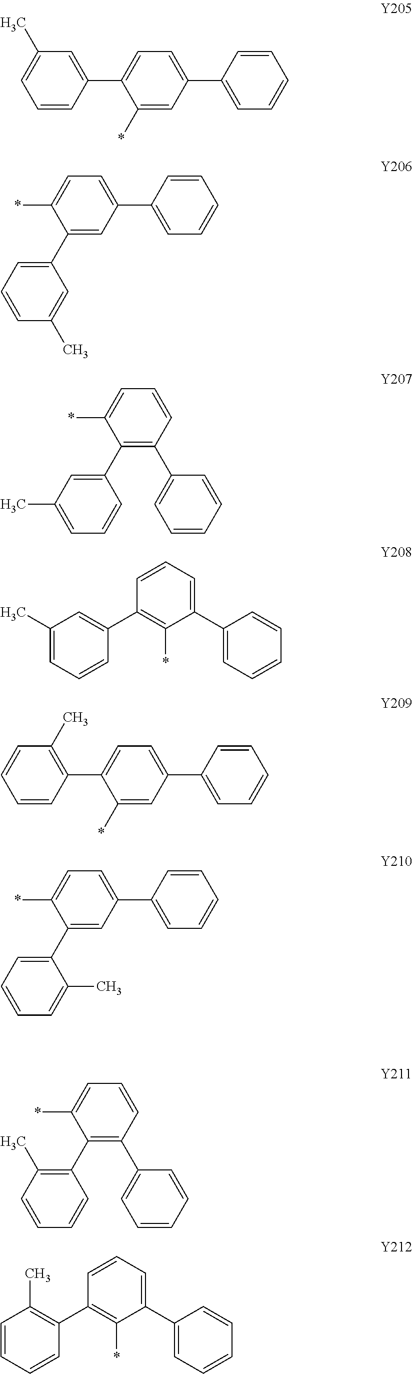

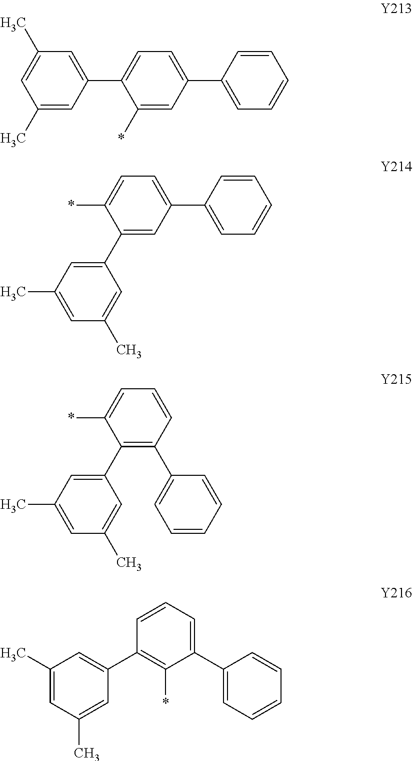

[chemical structure]

[chemical structure]

[chemical structure]

To suppress a transverse current, Ar¹ preferably denotes a group represented by any one of the formulae (Y1) to (Y298).

To suppress a transverse current, both Ar¹ and Ar² more preferably each independently denote a group represented by any one of the formulae (Y1) to (Y298).

To suppress a transverse current, both Ar¹ and Ar² still more preferably each independently denote a group represented by any one of the formulae (Y2) to (Y9), (Y11) to (Y18) and (Y21) to (Y298).

To suppress a transverse current, Ar¹ still more preferably denotes a group represented by any one of the formulae (Y25) to (Y46), (Y58) to (Y101), (Y103) to (Y124), (Y133) to (Y200), (Y225) to (Y256), (Y263) to (Y265) and (Y281) to (Y298).

To suppress a transverse current, still more preferably, Ar¹ denotes a group represented by any one of the formulae (Y25) to (Y46), (Y58) to (Y101), (Y103) to (Y124), (Y133) to (Y200), (Y225) to (Y256), (Y263) to (Y265) and (Y281) to (Y298), and Ar² denotes a group represented by any one of the formulae (Y1) to (Y298).

To suppress a transverse current, both Ar¹ and Ar² still more preferably each independently denote a group represented by any one of the formulae (Y25) to (Y46), (Y58) to (Y101), (Y103) to (Y124), (Y133) to (Y200), (Y225) to (Y256), (Y263) to (Y265) and (Y281) to (Y298).

In the formula (1),
specific examples of A include the formulae (a1) to (a262).

Specific examples of B include the formulae (b1) to (b309) and the formulae (c1) to (c1326). However, when A denotes one of the formulae (a1) to (a76) and (a213) to (a216), B is selected from the formulae (c1) to (c1326).

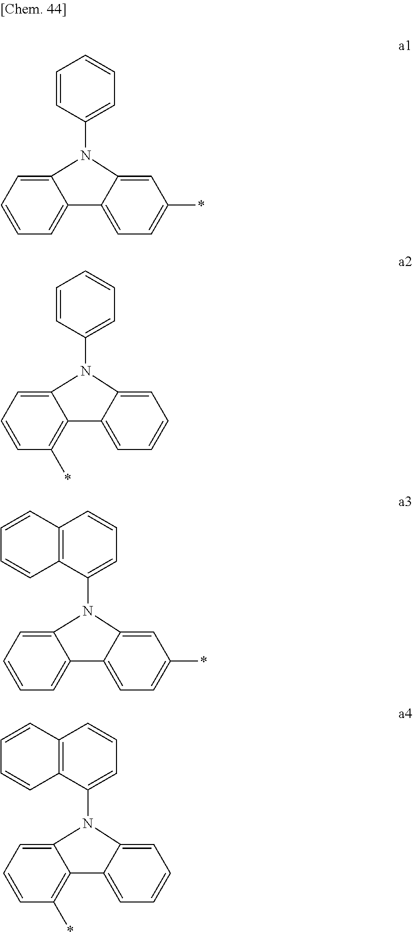

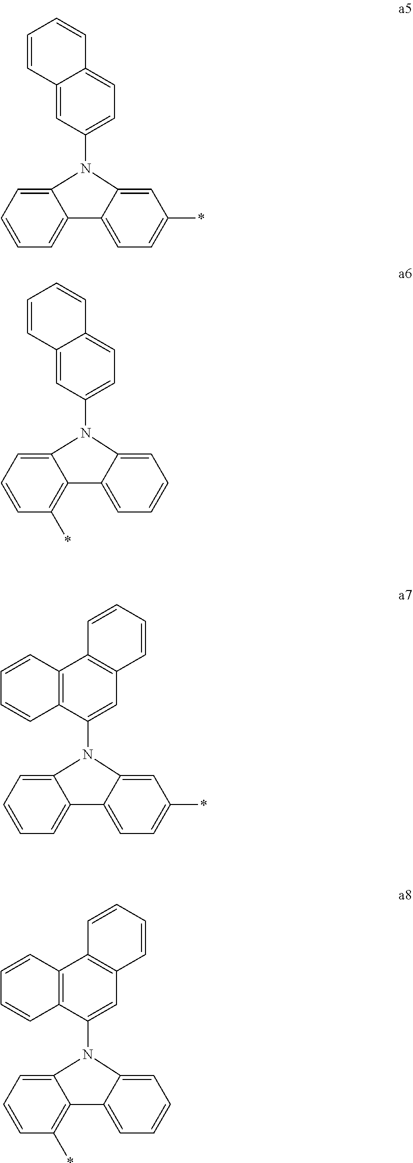

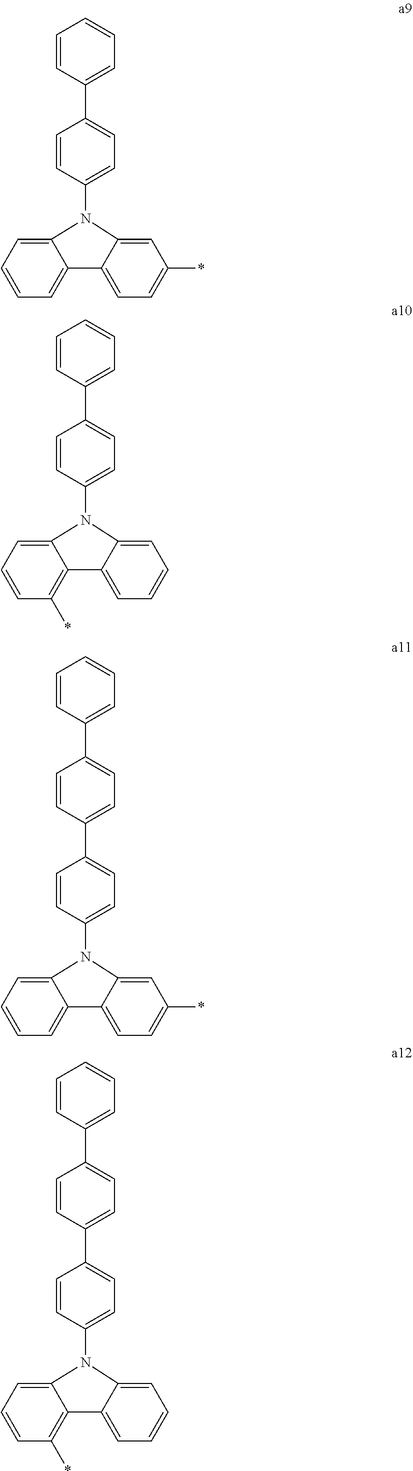

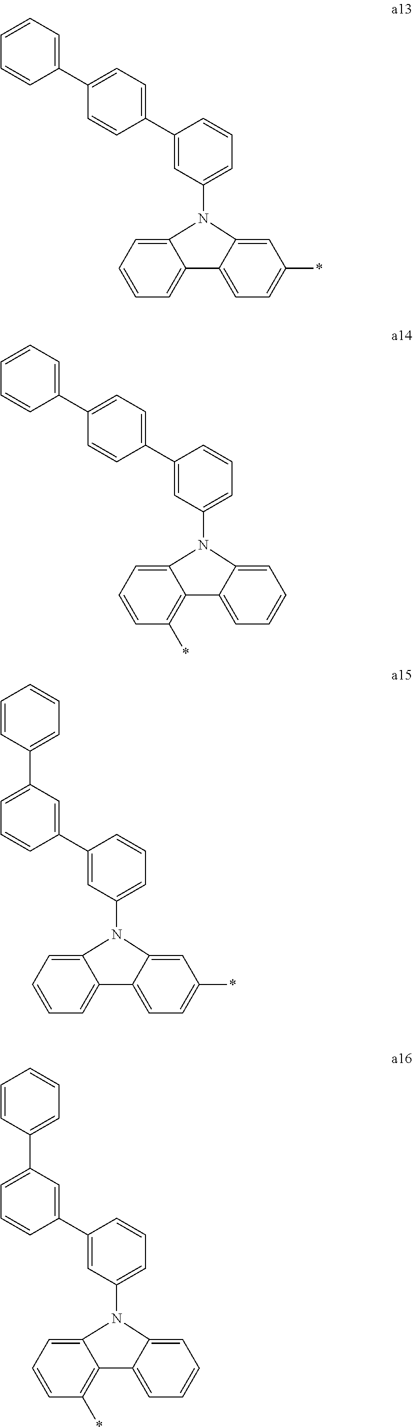 [SMILES: *c1ccc2c3ccccc3n(-c3cccc(-c4ccc(-c5ccccc5)cc4)c3)c2c1 ; *c1cccc2c1c1ccccc1n2-c1cccc(-c2ccc(-c3ccccc3)cc2)c1 ; *c1ccc2c3ccccc3n(-c3cccc(-c4cccc(-c5ccccc5)c4)c3)c2c1 ; *c1cccc2c1c1ccccc1n2-c1cccc(-c2cccc(-c3ccccc3)c2)c1]

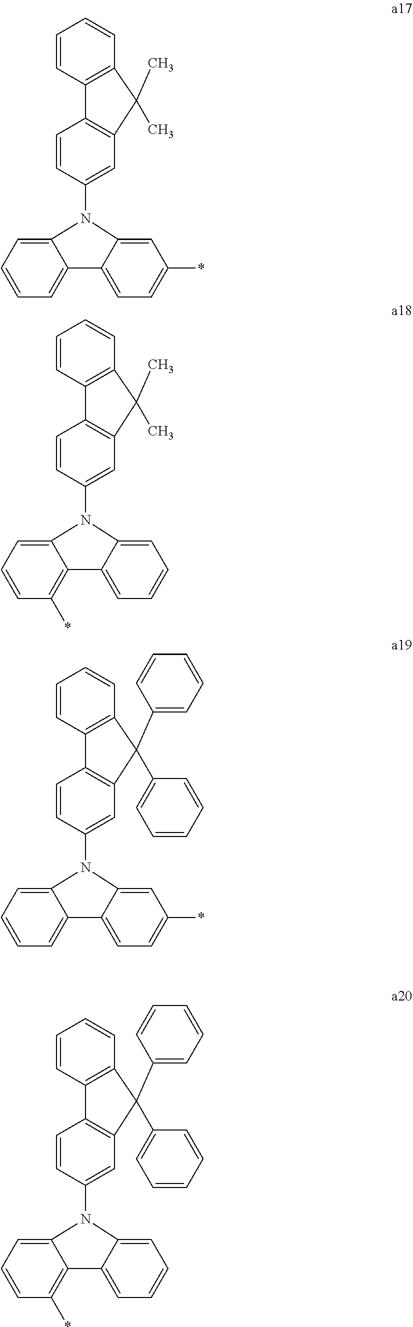

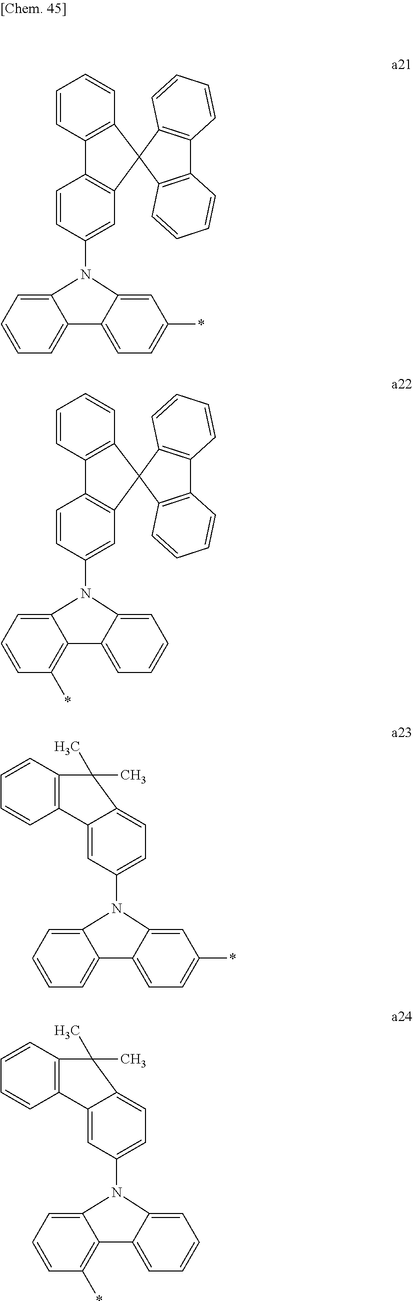

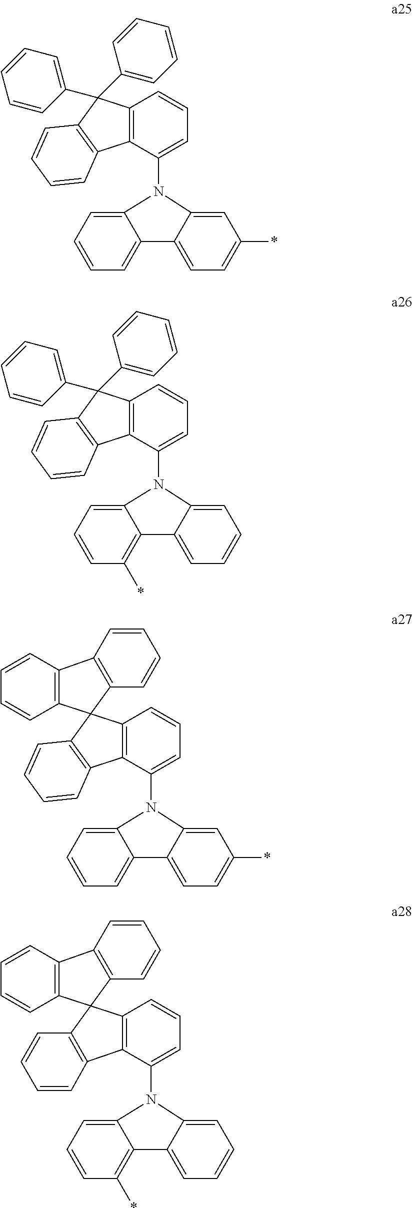

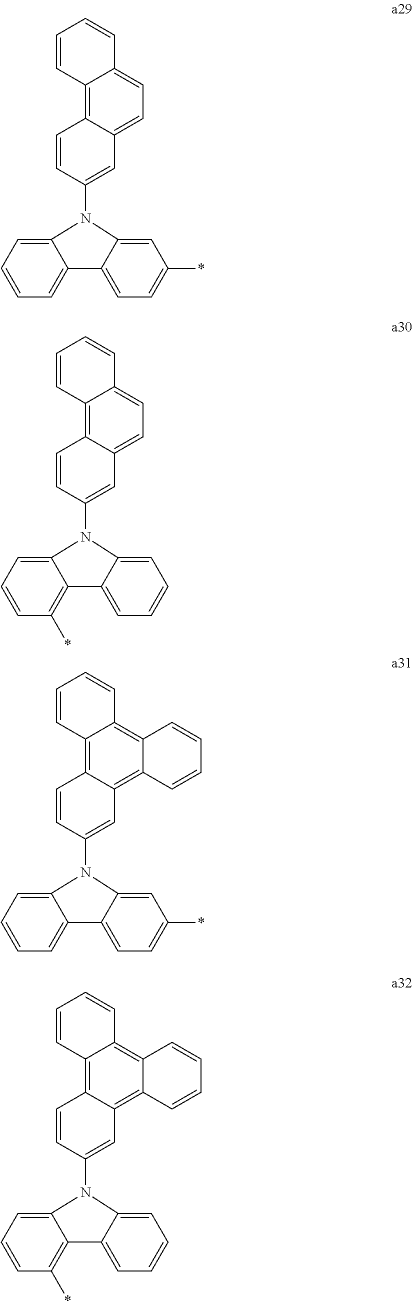

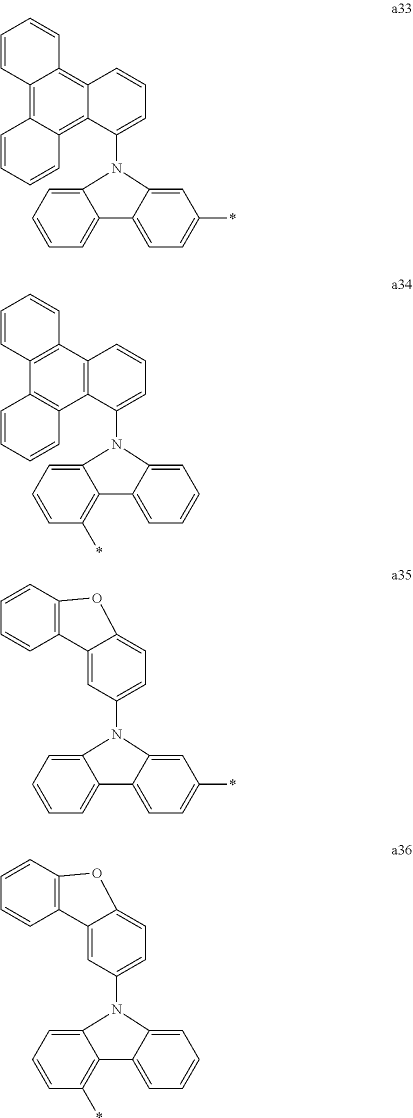

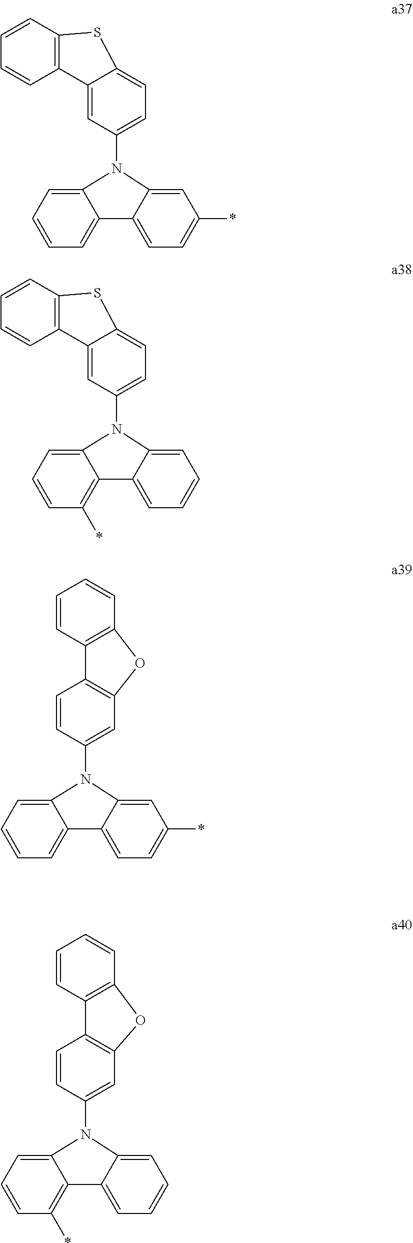

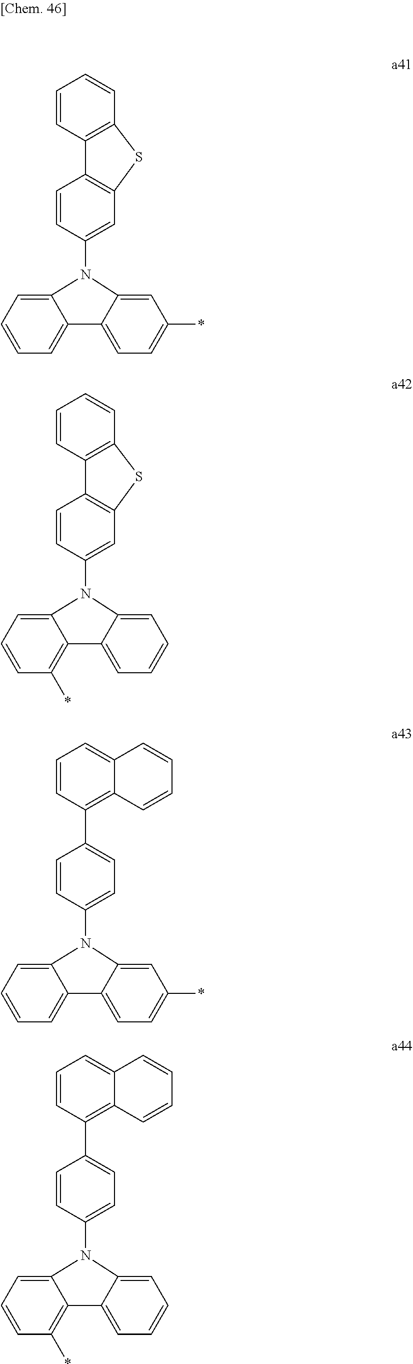

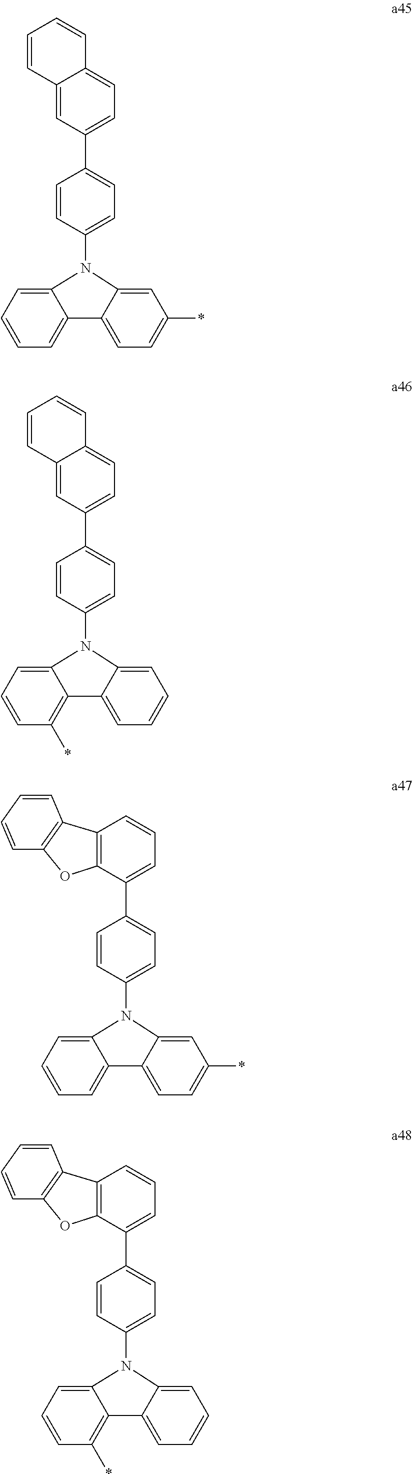

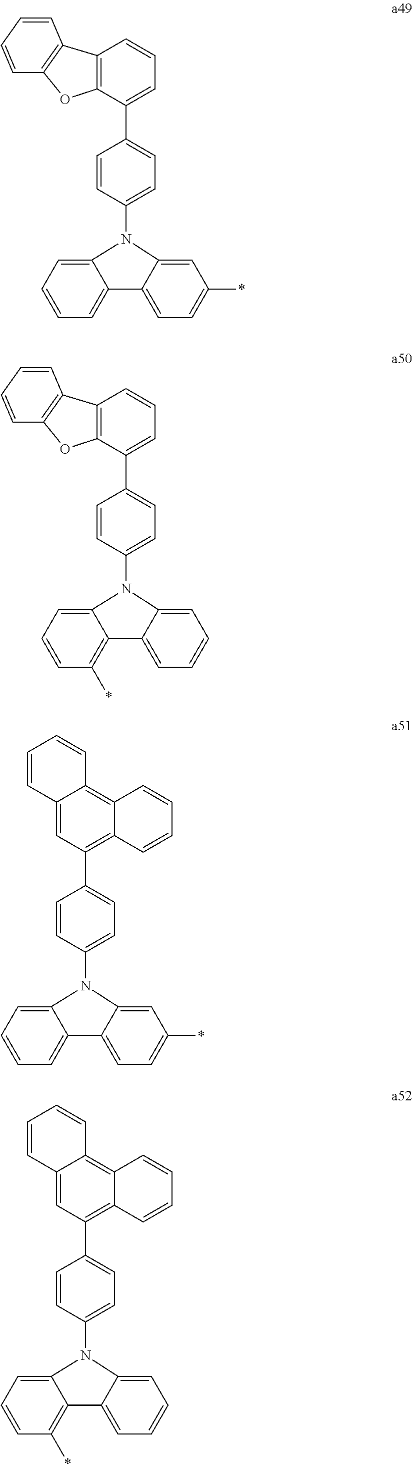

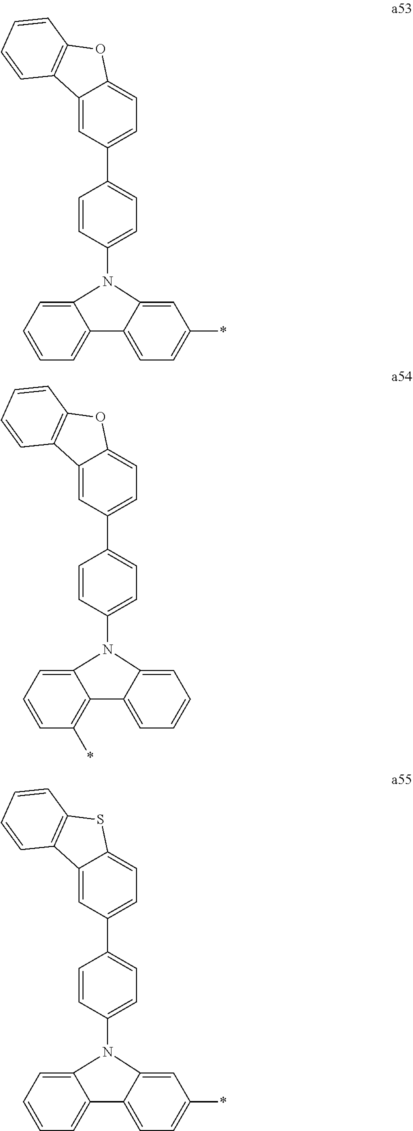

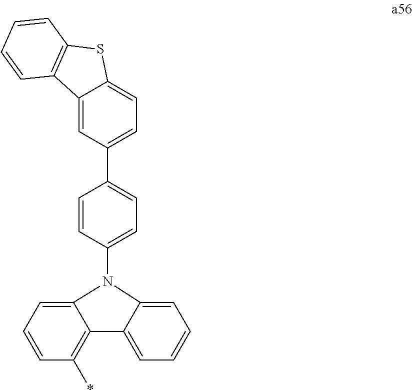 [SMILES: *c1cccc2c1c1ccccc1n2-c1ccc(-c2ccc3sc4ccccc4c3c2)cc1]

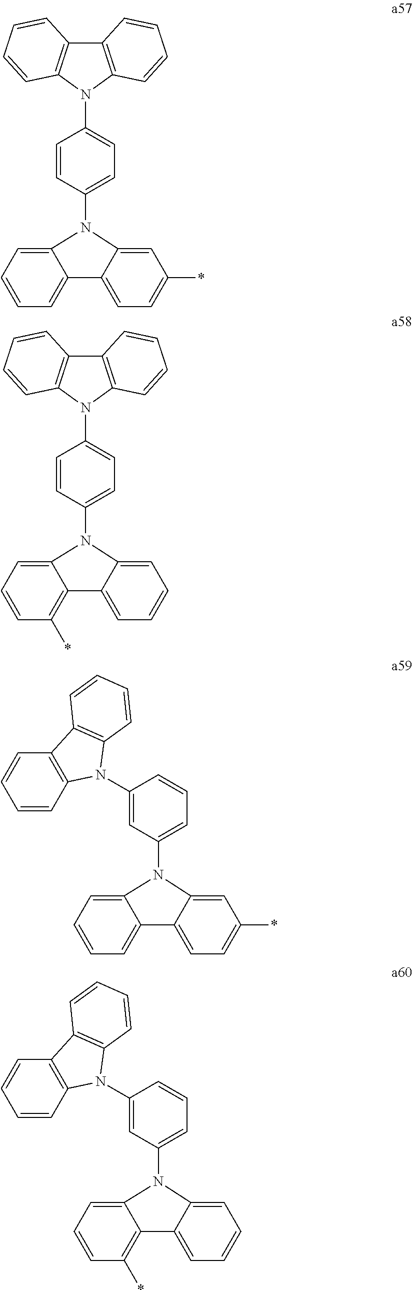

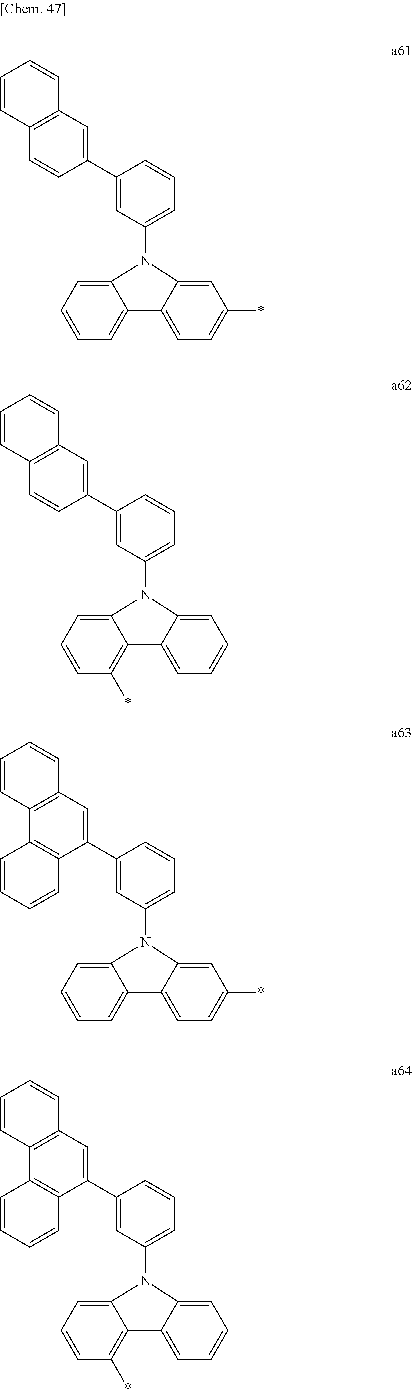 [SMILES: *c1ccc2c3ccccc3n(-c3cccc(-c4ccc5ccccc5c4)c3)c2c1 ; *c1cccc2c1c1ccccc1n2-c1cccc(-c2ccc3ccccc3c2)c1 ; *c1ccc2c3ccccc3n(-c3cccc(-c4cc5ccccc5c5ccccc45)c3)c2c1 ; *c1cccc2c1c1ccccc1n2-c1cccc(-c2cc3ccccc3c3ccccc23)c1]

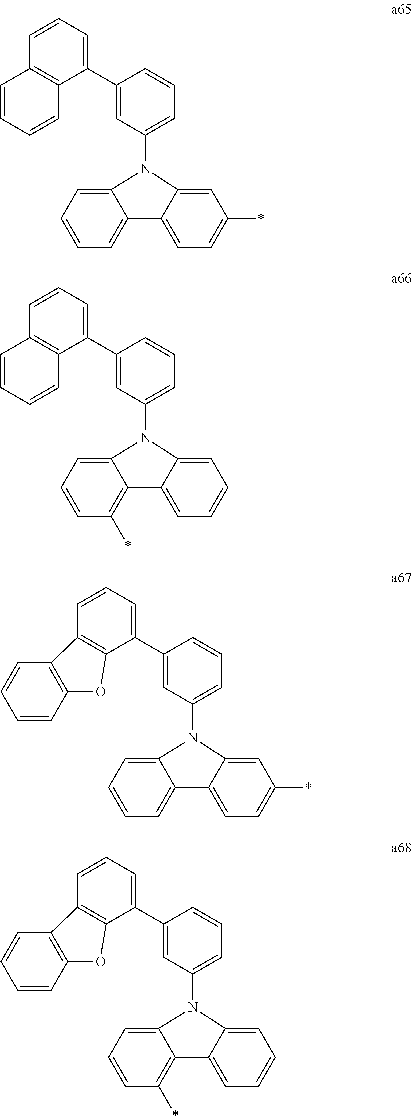

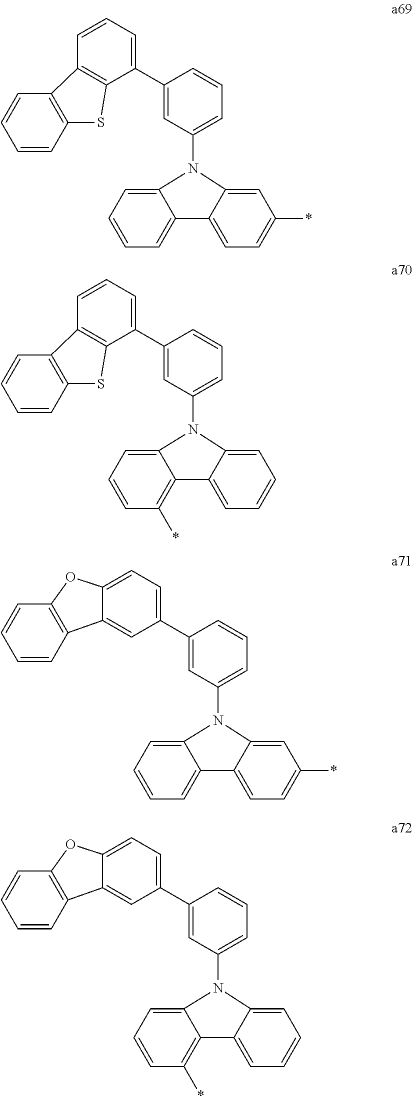

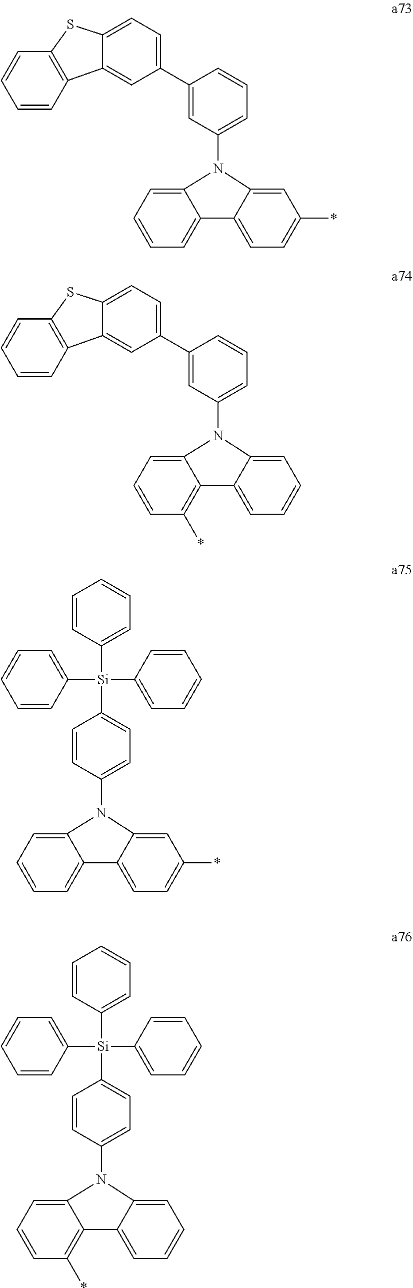

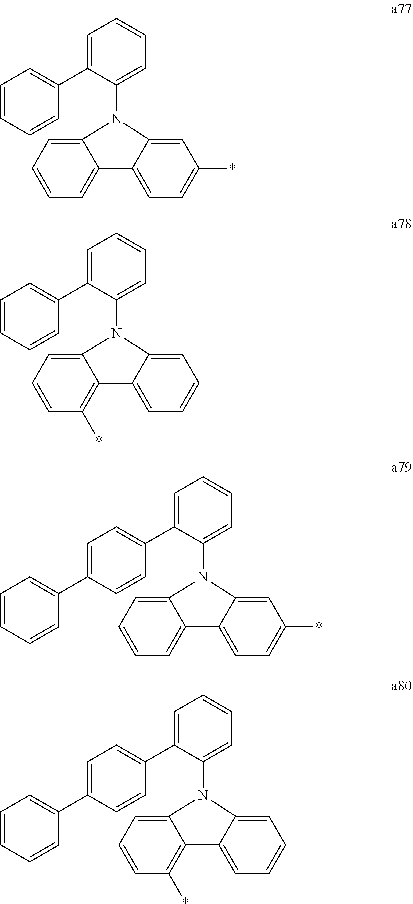 [SMILES: *c1ccc2c3ccccc3n(-c3ccccc3-c3ccccc3)c2c1 ; *c1cccc2c1c1ccccc1n2-c1ccccc1-c1ccccc1 ; *c1ccc2c3ccccc3n(-c3ccccc3-c3ccc(-c4ccccc4)cc3)c2c1 ; *c1cccc2c1c1ccccc1n2-c1ccccc1-c1ccc(-c2ccccc2)cc1]

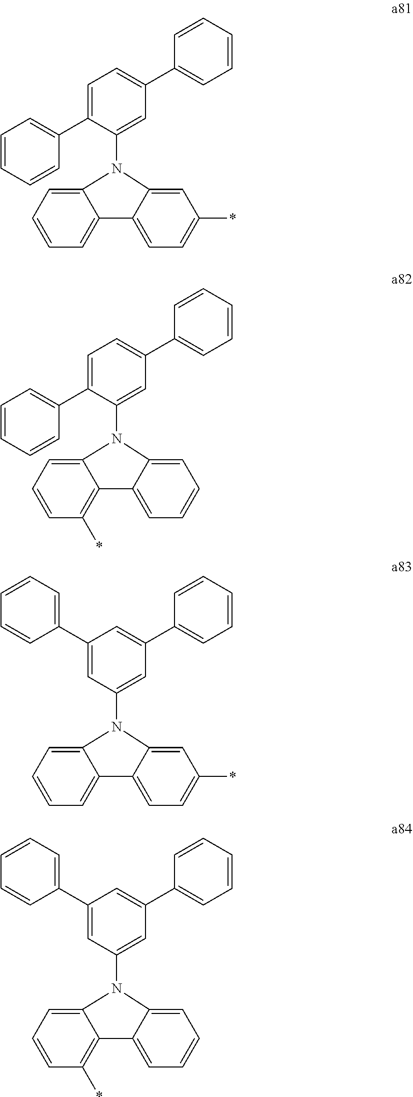

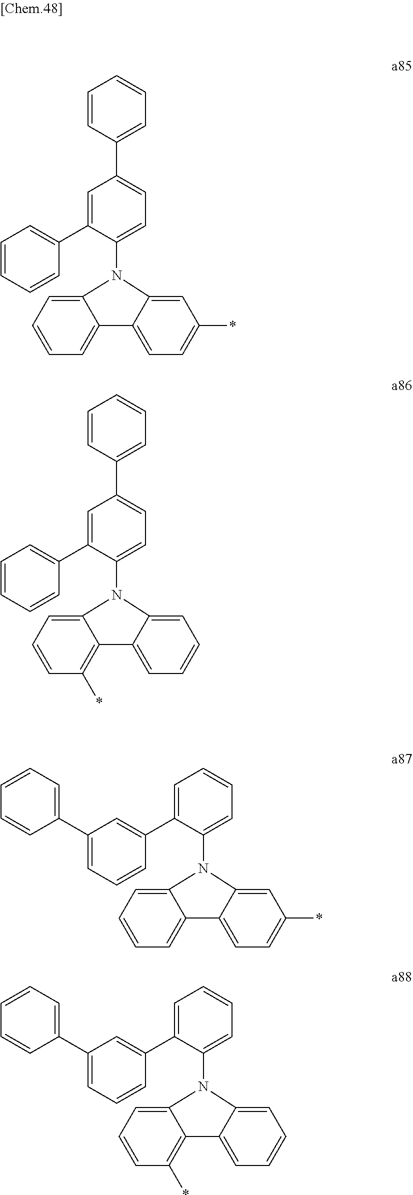 [SMILES: *c1ccc2c3ccccc3n(-c3ccc(-c4ccccc4)cc3-c3ccccc3)c2c1 ; *c1cccc2c1c1ccccc1n2-c1ccc(-c2ccccc2)cc1-c1ccccc1 ; *c1ccc2c3ccccc3n(-c3ccccc3-c3cccc(-c4ccccc4)c3)c2c1 ; *c1cccc2c1c1ccccc1n2-c1ccccc1-c1cccc(-c2ccccc2)c1]

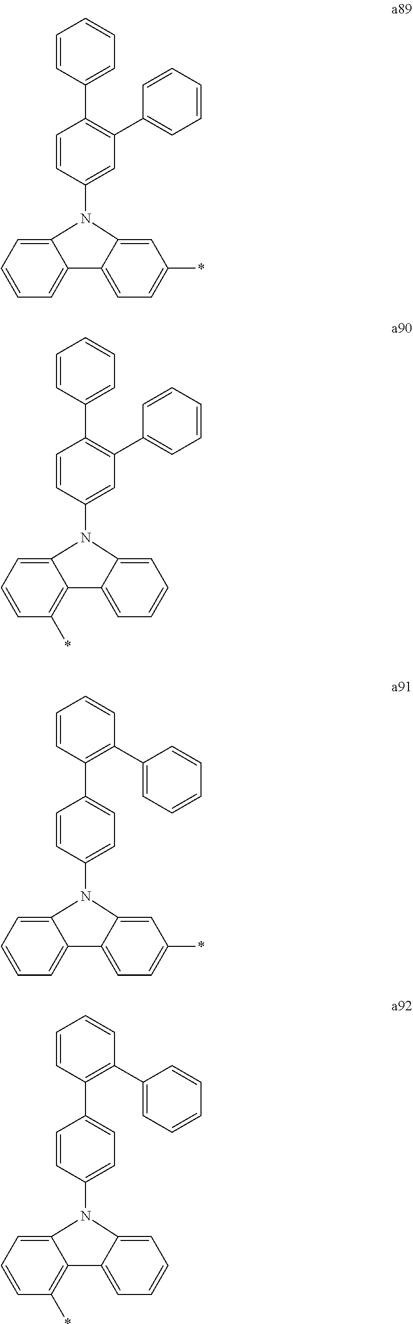 [SMILES: *c1ccc2c3ccccc3n(-c3ccc(-c4ccccc4)c(-c4ccccc4)c3)c2c1 ; *c1cccc2c1c1ccccc1n2-c1ccc(-c2ccccc2)c(-c2ccccc2)c1 ; *c1ccc2c3ccccc3n(-c3ccc(-c4ccccc4-c4ccccc4)cc3)c2c1 ; *c1cccc2c1c1ccccc1n2-c1ccc(-c2ccccc2-c2ccccc2)cc1]

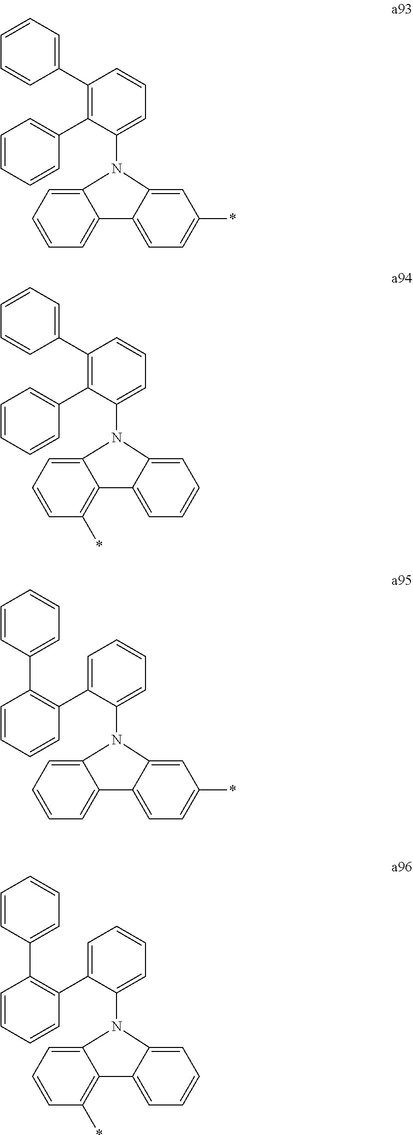

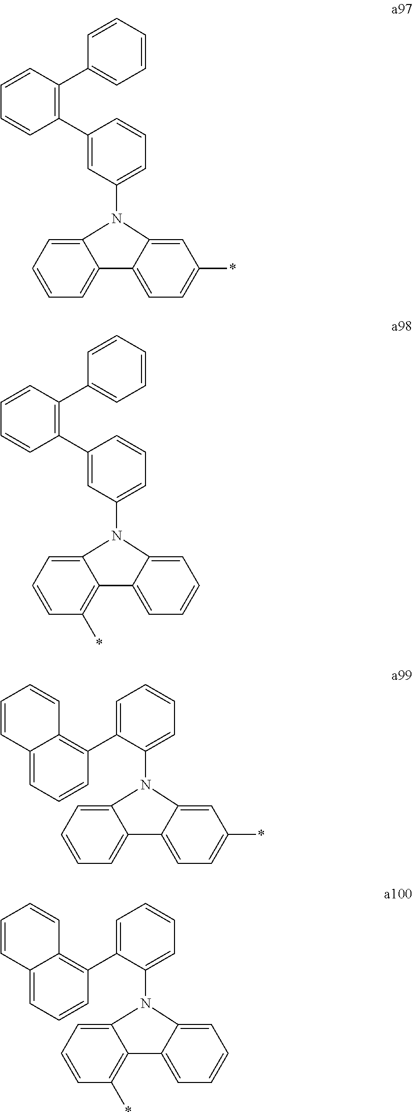 [SMILES: *c1ccc2c3ccccc3n(-c3cccc(-c4ccccc4-c4ccccc4)c3)c2c1 ; *c1cccc2c1c1ccccc1n2-c1cccc(-c2ccccc2-c2ccccc2)c1 ; *c1ccc2c3ccccc3n(-c3ccccc3-c3cccc4ccccc34)c2c1 ; *c1cccc2c1c1ccccc1n2-c1ccccc1-c1cccc2ccccc12]

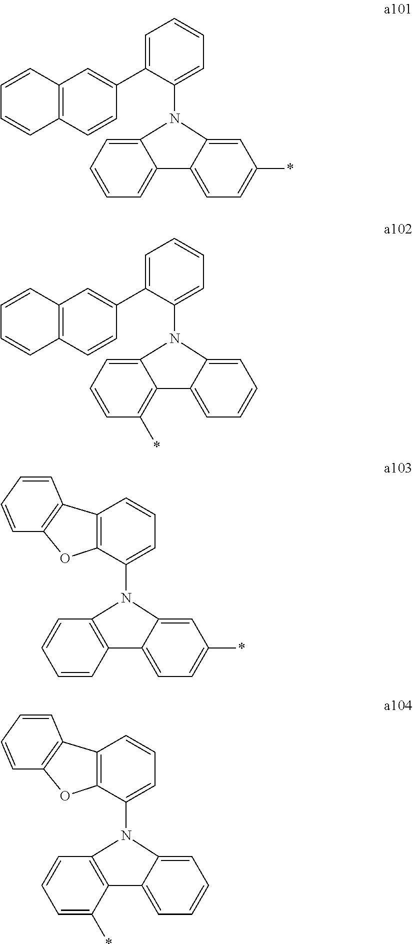

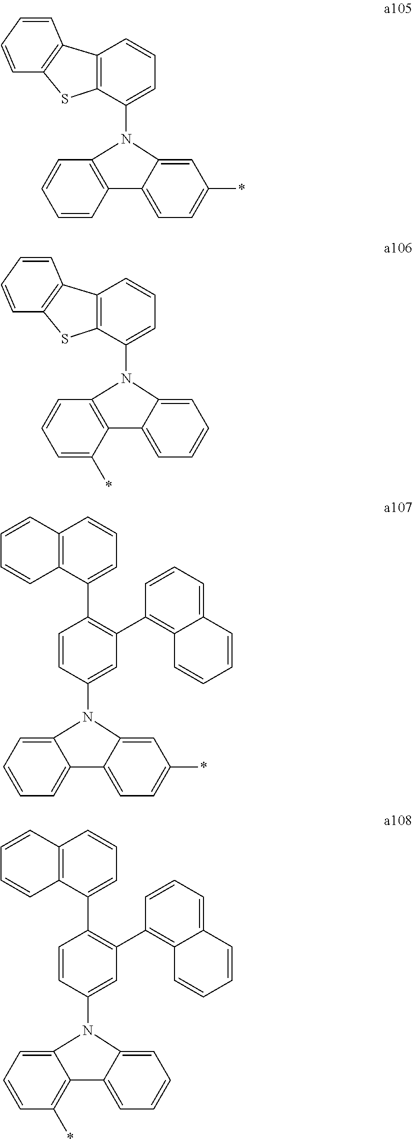

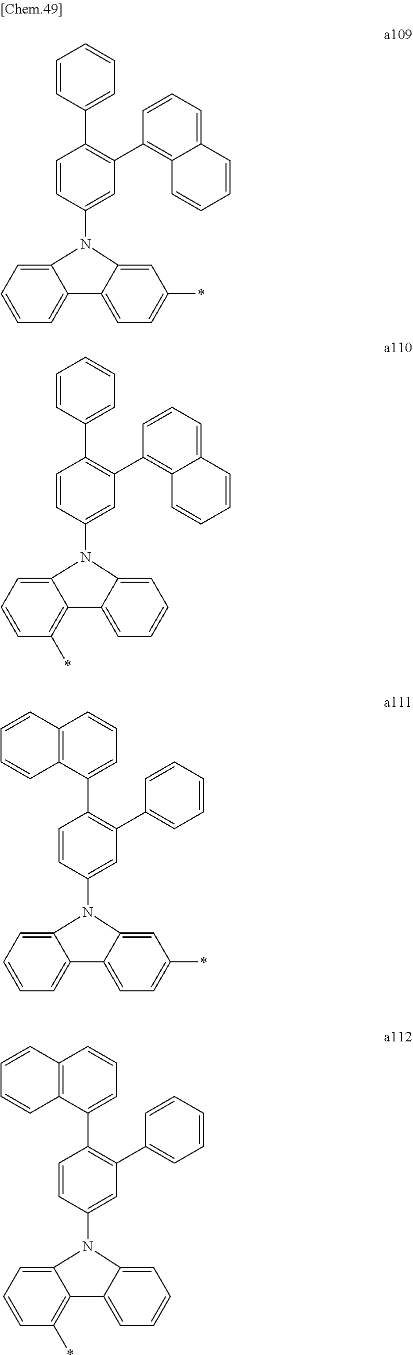 [SMILES: *c1ccc2c3ccccc3n(-c3ccc(-c4ccccc4)c(-c4cccc5ccccc45)c3)c2c1 ; *c1cccc2c1c1ccccc1n2-c1ccc(-c2ccccc2)c(-c2cccc3ccccc23)c1 ; *c1ccc2c3ccccc3n(-c3ccc(-c4cccc5ccccc45)c(-c4ccccc4)c3)c2c1 ; *c1cccc2c1c1ccccc1n2-c1ccc(-c2cccc3ccccc23)c(-c2ccccc2)c1]

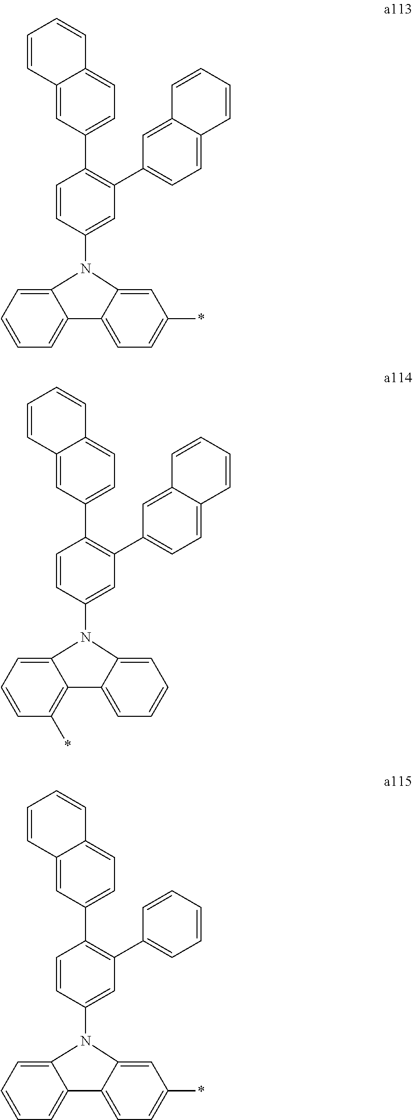

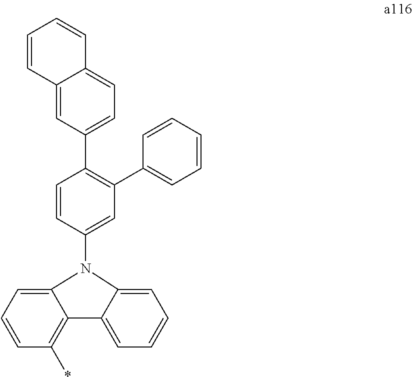 [SMILES: *c1cccc2c1c1ccccc1n2-c1ccc(-c2ccc3ccccc3c2)c(-c2ccccc2)c1]

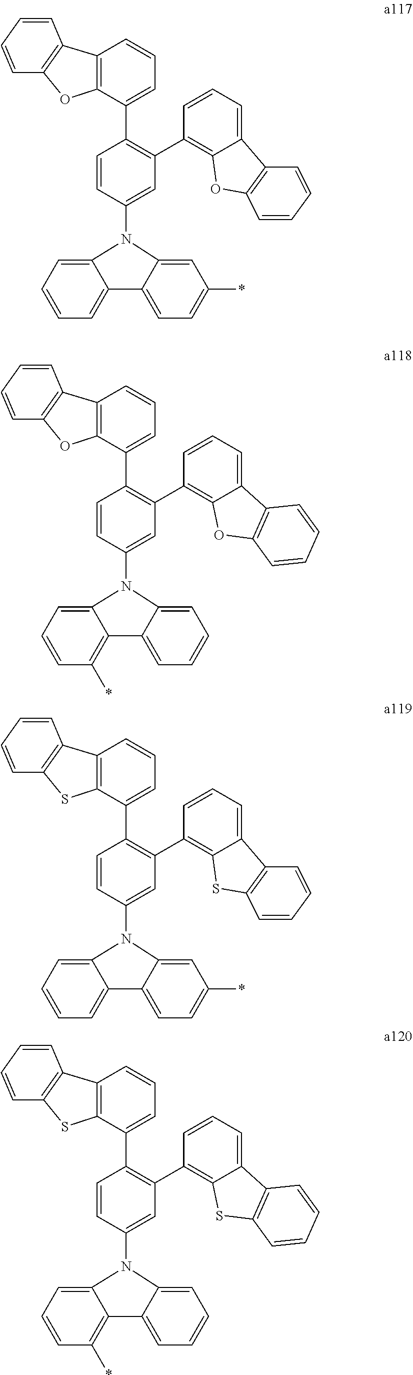

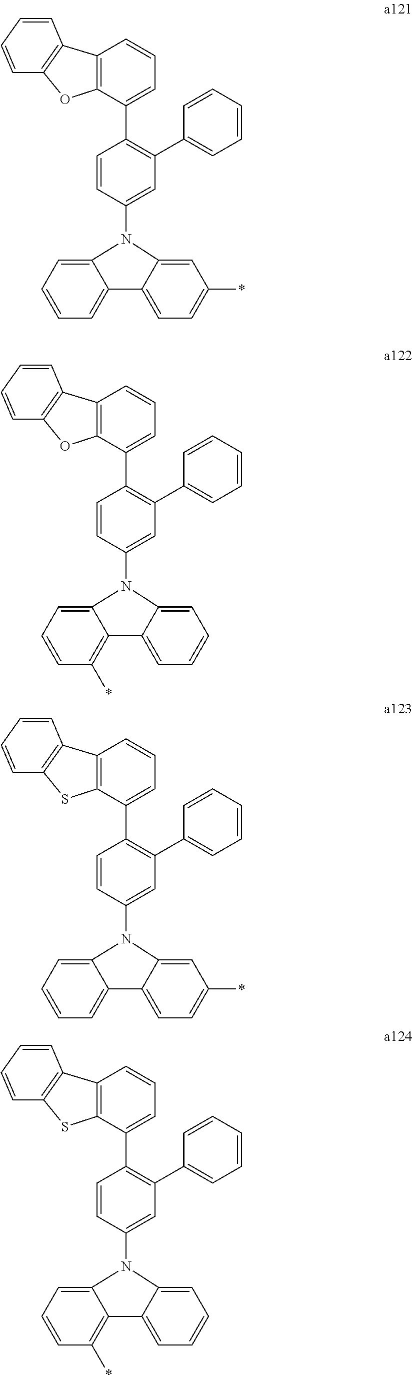

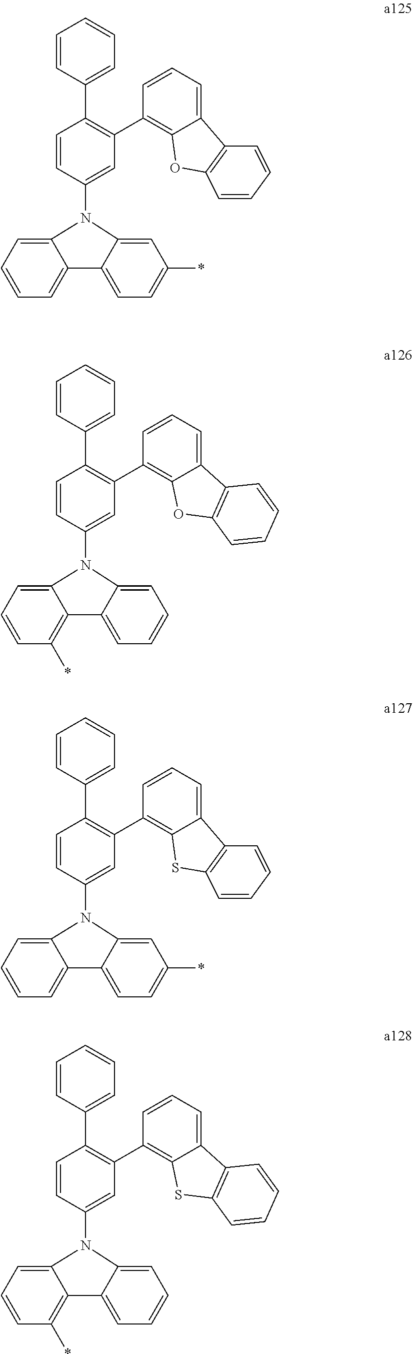 [SMILES: *c1ccc2c3ccccc3n(-c3ccc(-c4ccccc4)c(-c4cccc5c4oc4ccccc45)c3)c2c1 ; *c1cccc2c1c1ccccc1n2-c1ccc(-c2ccccc2)c(-c2cccc3c2oc2ccccc23)c1 ; *c1ccc2c3ccccc3n(-c3ccc(-c4ccccc4)c(-c4cccc5c4sc4ccccc45)c3)c2c1 ; *c1cccc2c1c1ccccc1n2-c1ccc(-c2ccccc2)c(-c2cccc3c2sc2ccccc23)c1]

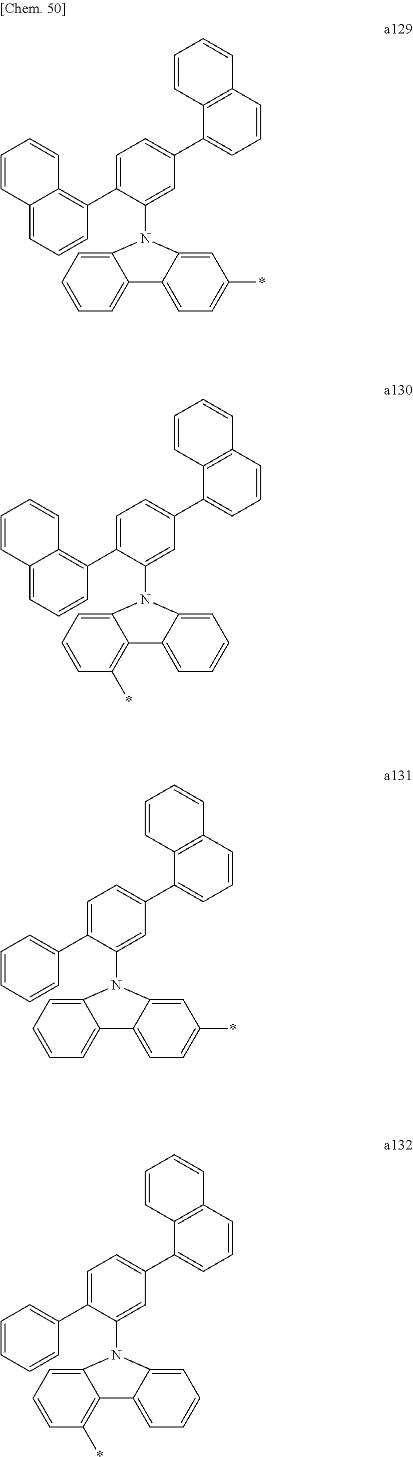 [SMILES: *c1ccc2c3ccccc3n(-c3cc(-c4cccc5ccccc45)ccc3-c3cccc4ccccc34)c2c1 ; *c1cccc2c1c1ccccc1n2-c1cc(-c2cccc3ccccc23)ccc1-c1cccc2ccccc12 ; *c1ccc2c3ccccc3n(-c3cc(-c4cccc5ccccc45)ccc3-c3ccccc3)c2c1 ; *c1cccc2c1c1ccccc1n2-c1cc(-c2cccc3ccccc23)ccc1-c1ccccc1]

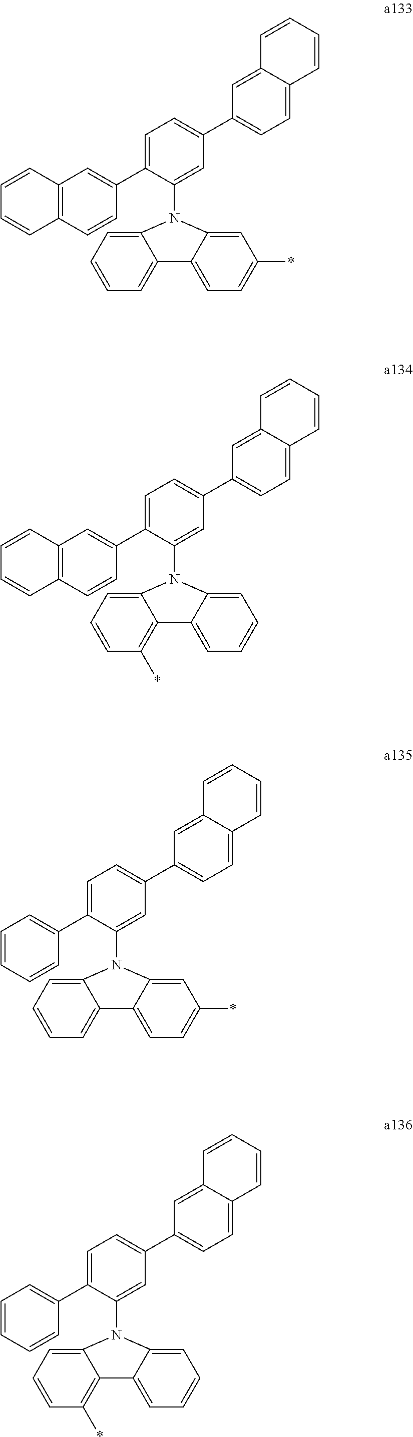 [SMILES: *c1ccc2c3ccccc3n(-c3cc(-c4ccc5ccccc5c4)ccc3-c3ccc4ccccc4c3)c2c1 ; *c1cccc2c1c1ccccc1n2-c1cc(-c2ccc3ccccc3c2)ccc1-c1ccc2ccccc2c1 ; *c1ccc2c3ccccc3n(-c3cc(-c4ccc5ccccc5c4)ccc3-c3ccccc3)c2c1 ; *c1cccc2c1c1ccccc1n2-c1cc(-c2ccc3ccccc3c2)ccc1-c1ccccc1]

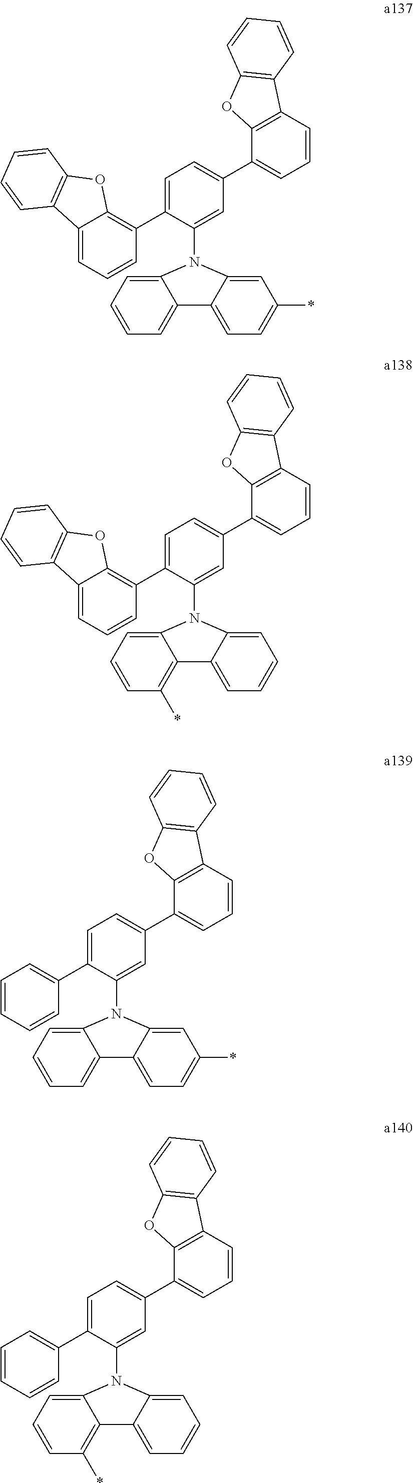

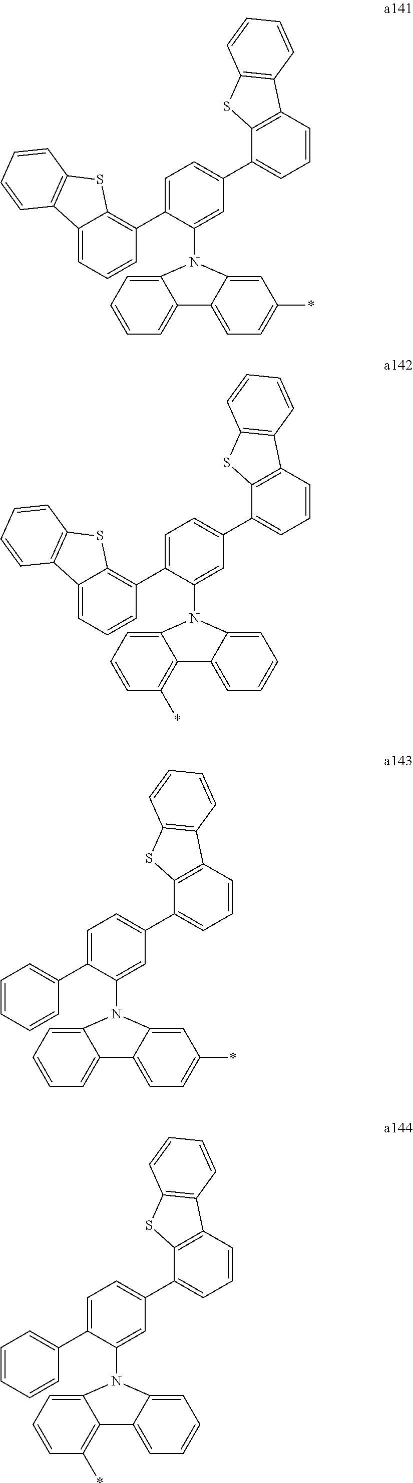

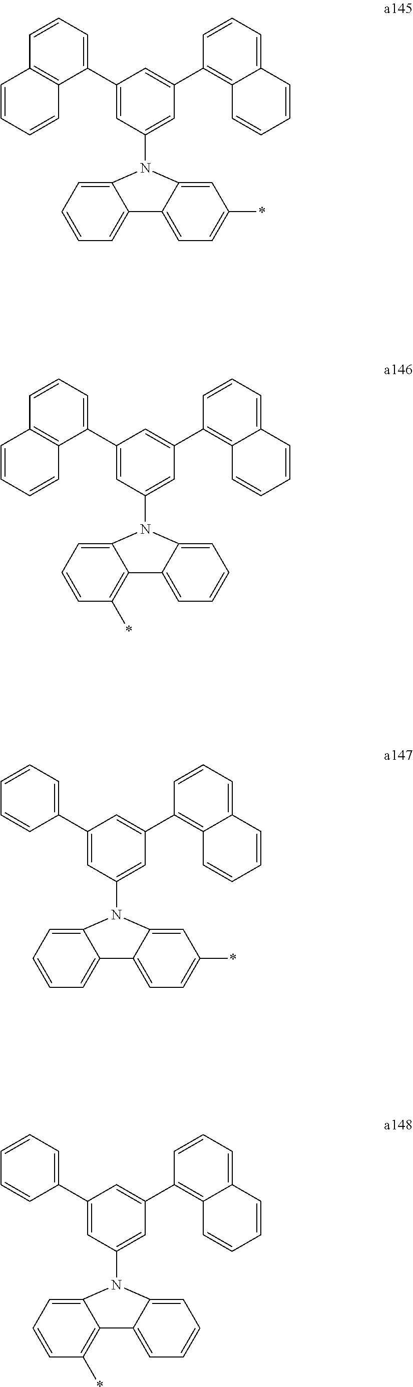 [SMILES: *c1ccc2c3ccccc3n(-c3cc(-c4cccc5ccccc45)cc(-c4cccc5ccccc45)c3)c2c1 ; *c1cccc2c1c1ccccc1n2-c1cc(-c2cccc3ccccc23)cc(-c2cccc3ccccc23)c1 ; *c1ccc2c3ccccc3n(-c3cc(-c4ccccc4)cc(-c4cccc5ccccc45)c3)c2c1 ; *c1cccc2c1c1ccccc1n2-c1cc(-c2ccccc2)cc(-c2cccc3ccccc23)c1]

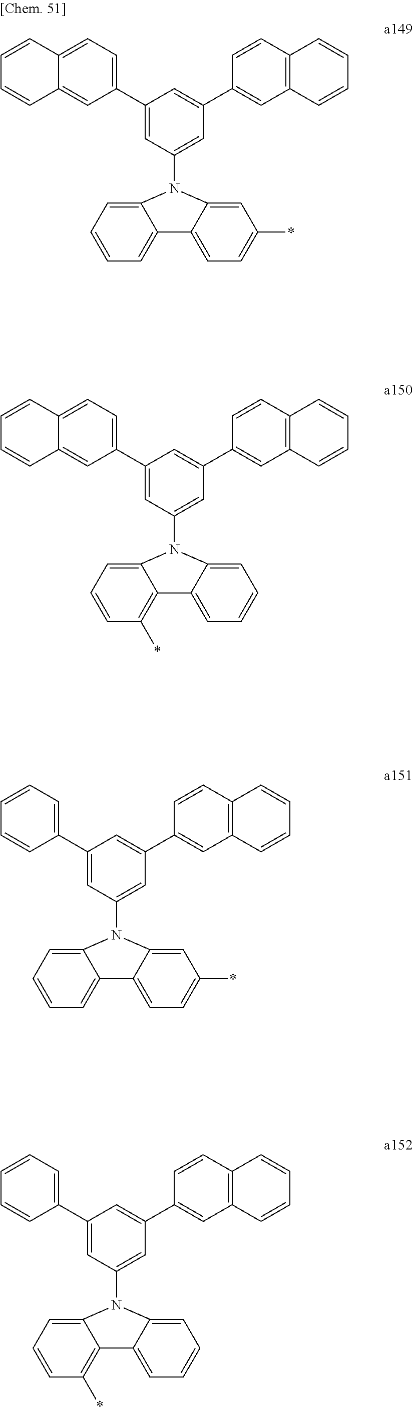 [SMILES: *c1ccc2c3ccccc3n(-c3cc(-c4ccc5ccccc5c4)cc(-c4ccc5ccccc5c4)c3)c2c1 ; *c1cccc2c1c1ccccc1n2-c1cc(-c2ccc3ccccc3c2)cc(-c2ccc3ccccc3c2)c1 ; *c1ccc2c3ccccc3n(-c3cc(-c4ccccc4)cc(-c4ccc5ccccc5c4)c3)c2c1 ; *c1cccc2c1c1ccccc1n2-c1cc(-c2ccccc2)cc(-c2ccc3ccccc3c2)c1]

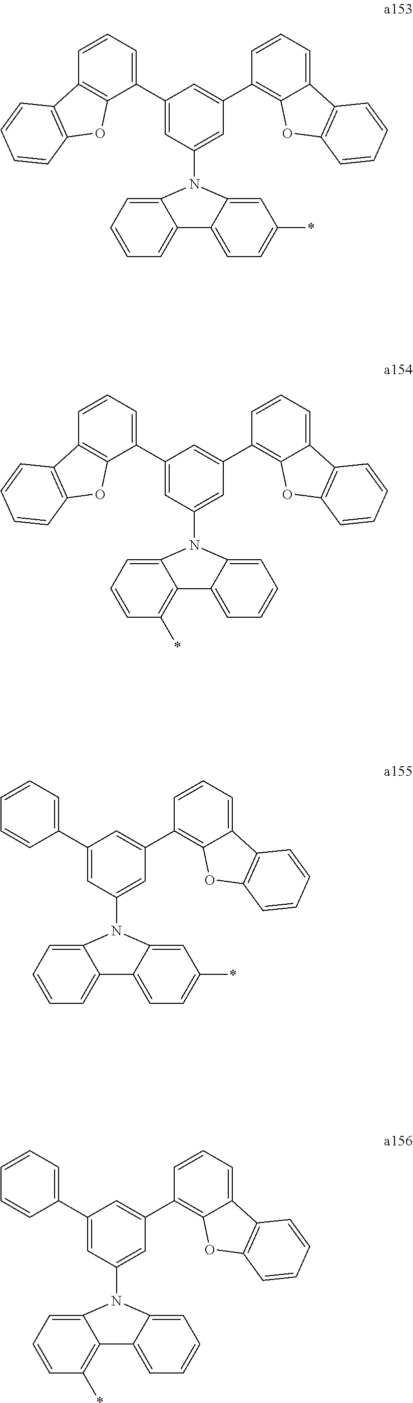 [SMILES: *c1ccc2c3ccccc3n(-c3cc(-c4cccc5c4oc4ccccc45)cc(-c4cccc5c4oc4ccccc45)c3)c2c1 ; *c1cccc2c1c1ccccc1n2-c1cc(-c2cccc3c2oc2ccccc23)cc(-c2cccc3c2oc2ccccc23)c1 ; *c1ccc2c3ccccc3n(-c3cc(-c4ccccc4)cc(-c4cccc5c4oc4ccccc45)c3)c2c1 ; *c1cccc2c1c1ccccc1n2-c1cc(-c2ccccc2)cc(-c2cccc3c2oc2ccccc23)c1]

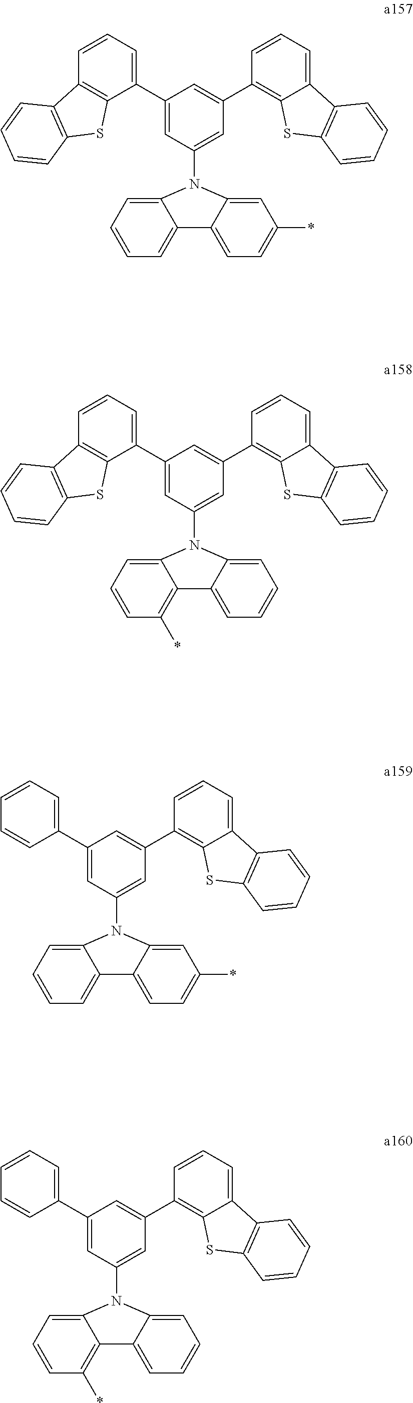 [SMILES: *c1ccc2c3ccccc3n(-c3cc(-c4cccc5c4sc4ccccc45)cc(-c4cccc5c4sc4ccccc45)c3)c2c1 ; *c1cccc2c1c1ccccc1n2-c1cc(-c2cccc3c2sc2ccccc23)cc(-c2cccc3c2sc2ccccc23)c1 ; *c1ccc2c3ccccc3n(-c3cc(-c4ccccc4)cc(-c4cccc5c4sc4ccccc45)c3)c2c1 ; *c1cccc2c1c1ccccc1n2-c1cc(-c2ccccc2)cc(-c2cccc3c2sc2ccccc23)c1]

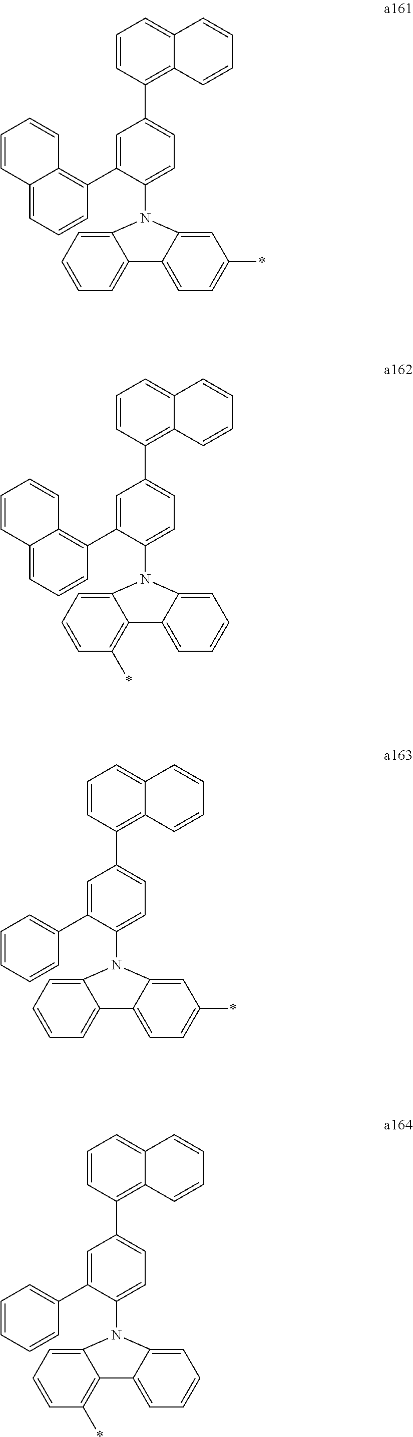 [SMILES: *c1ccc2c3ccccc3n(-c3ccc(-c4cccc5ccccc45)cc3-c3cccc4ccccc34)c2c1 ; *c1cccc2c1c1ccccc1n2-c1ccc(-c2cccc3ccccc23)cc1-c1cccc2ccccc12 ; *c1ccc2c3ccccc3n(-c3ccc(-c4cccc5ccccc45)cc3-c3ccccc3)c2c1 ; *c1cccc2c1c1ccccc1n2-c1ccc(-c2cccc3ccccc23)cc1-c1ccccc1]

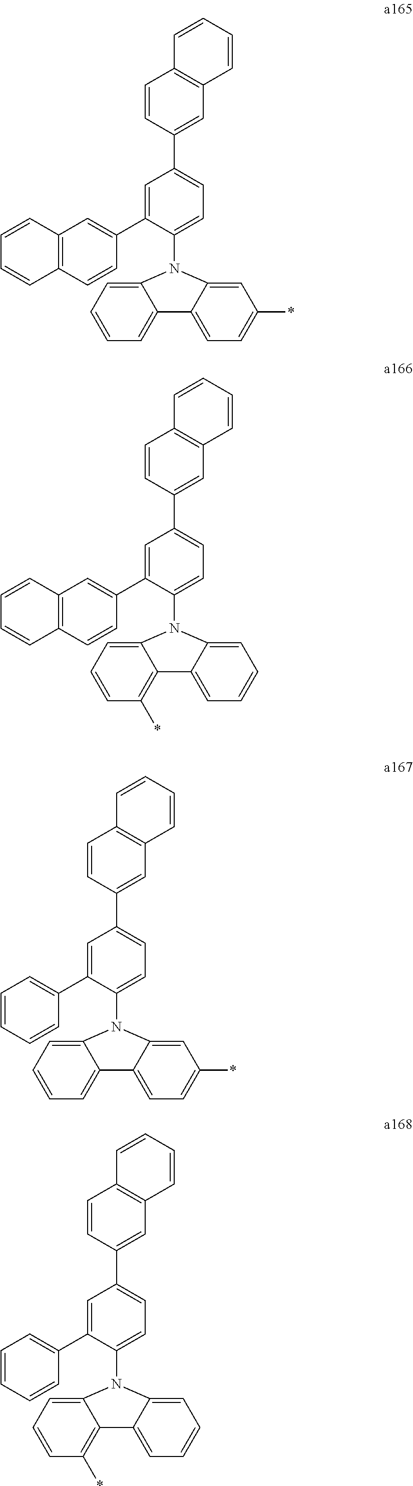

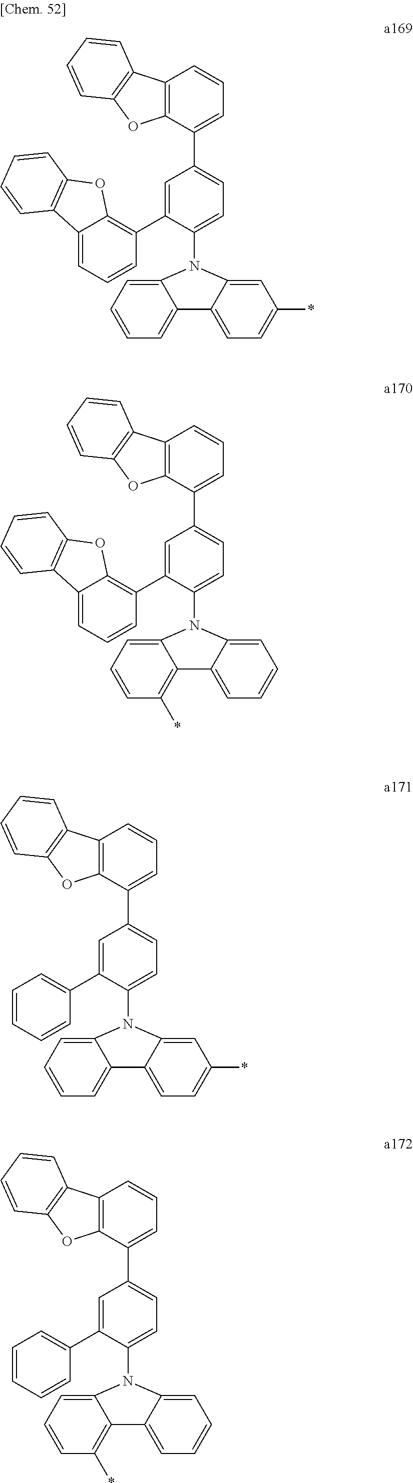 [SMILES: *c1ccc2c3ccccc3n(-c3ccc(-c4cccc5c4oc4ccccc45)cc3-c3cccc4c3oc3ccccc34)c2c1 ; *c1cccc2c1c1ccccc1n2-c1ccc(-c2cccc3c2oc2ccccc23)cc1-c1cccc2c1oc1ccccc12 ; *c1ccc2c3ccccc3n(-c3ccc(-c4cccc5c4oc4ccccc45)cc3-c3ccccc3)c2c1 ; *c1cccc2c1c1ccccc1n2-c1ccc(-c2cccc3c2oc2ccccc23)cc1-c1ccccc1]

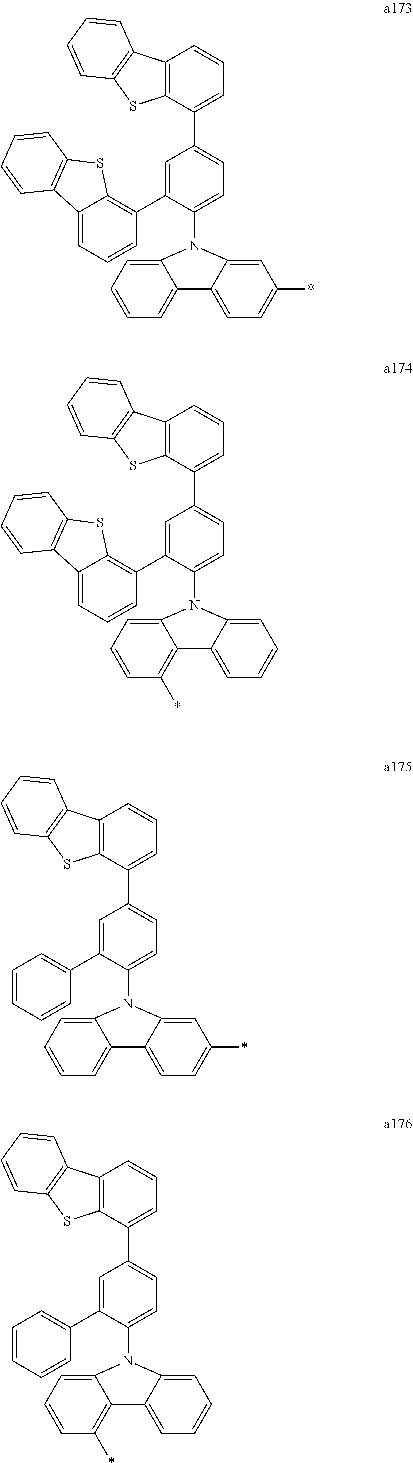 [SMILES: *c1ccc2c3ccccc3n(-c3ccc(-c4cccc5c4sc4ccccc45)cc3-c3cccc4c3sc3ccccc34)c2c1 ; *c1cccc2c1c1ccccc1n2-c1ccc(-c2cccc3c2sc2ccccc23)cc1-c1cccc2c1sc1ccccc12 ; *c1ccc2c3ccccc3n(-c3ccc(-c4cccc5c4sc4ccccc45)cc3-c3ccccc3)c2c1 ; *c1cccc2c1c1ccccc1n2-c1ccc(-c2cccc3c2sc2ccccc23)cc1-c1ccccc1]

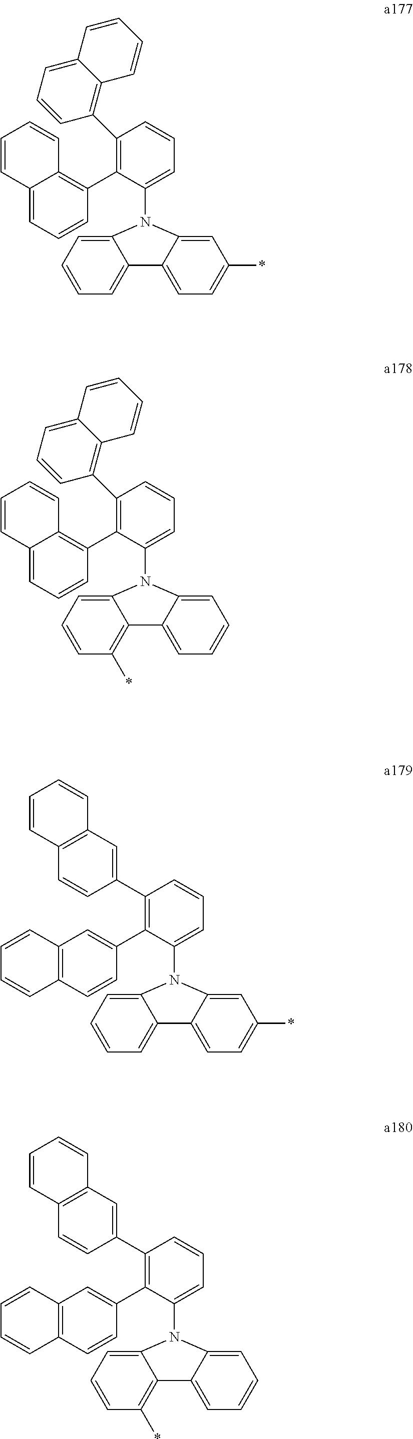 [SMILES: *c1ccc2c3ccccc3n(-c3cccc(-c4cccc5ccccc45)c3-c3cccc4ccccc34)c2c1 ; *c1cccc2c1c1ccccc1n2-c1cccc(-c2cccc3ccccc23)c1-c1cccc2ccccc12 ; *c1ccc2c3ccccc3n(-c3cccc(-c4ccc5ccccc5c4)c3-c3ccc4ccccc4c3)c2c1 ; *c1cccc2c1c1ccccc1n2-c1cccc(-c2ccc3ccccc3c2)c1-c1ccc2ccccc2c1]

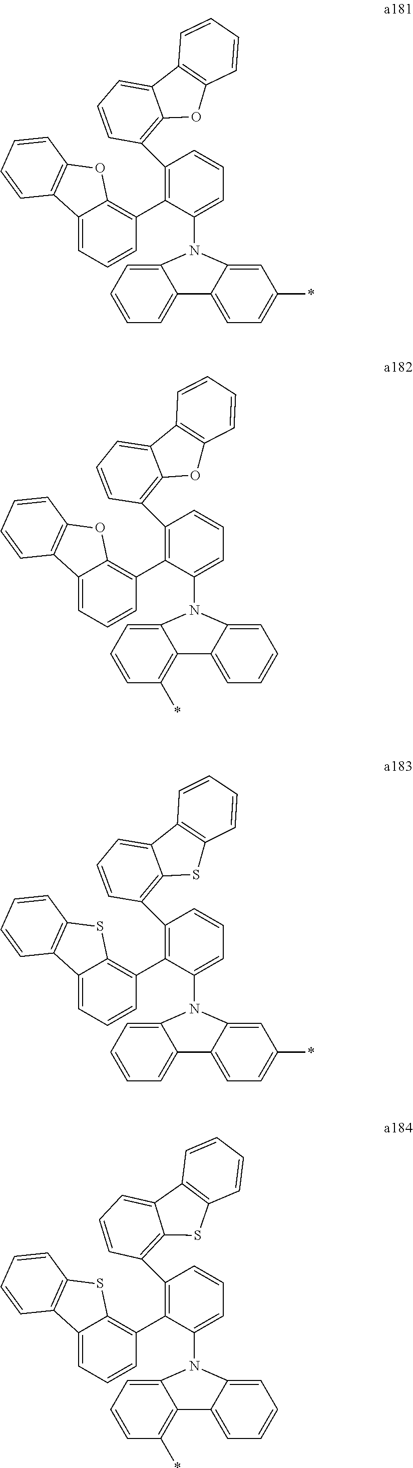 [SMILES: *c1ccc2c3ccccc3n(-c3cccc(-c4cccc5c4oc4ccccc45)c3-c3cccc4c3oc3ccccc34)c2c1 ; *c1cccc2c1c1ccccc1n2-c1cccc(-c2cccc3c2oc2ccccc23)c1-c1cccc2c1oc1ccccc12 ; *c1ccc2c3ccccc3n(-c3cccc(-c4cccc5c4sc4ccccc45)c3-c3cccc4c3sc3ccccc34)c2c1 ; *c1cccc2c1c1ccccc1n2-c1cccc(-c2cccc3c2sc2ccccc23)c1-c1cccc2c1sc1ccccc12]

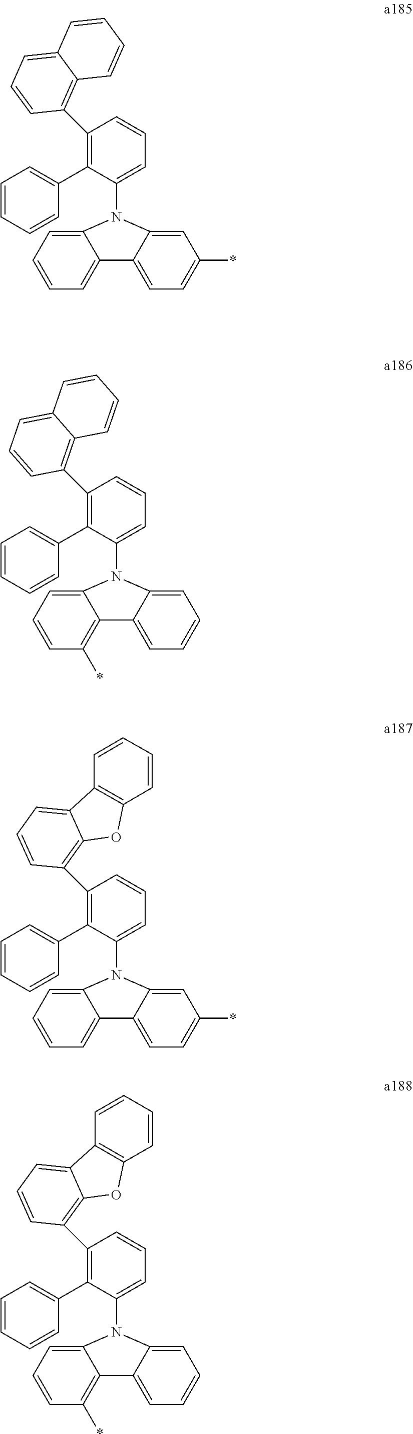 [SMILES: *c1ccc2c3ccccc3n(-c3cccc(-c4cccc5ccccc45)c3-c3ccccc3)c2c1 ; *c1cccc2c1c1ccccc1n2-c1cccc(-c2cccc3ccccc23)c1-c1ccccc1 ; *c1ccc2c3ccccc3n(-c3cccc(-c4cccc5c4oc4ccccc45)c3-c3ccccc3)c2c1 ; *c1cccc2c1c1ccccc1n2-c1cccc(-c2cccc3c2oc2ccccc23)c1-c1ccccc1]

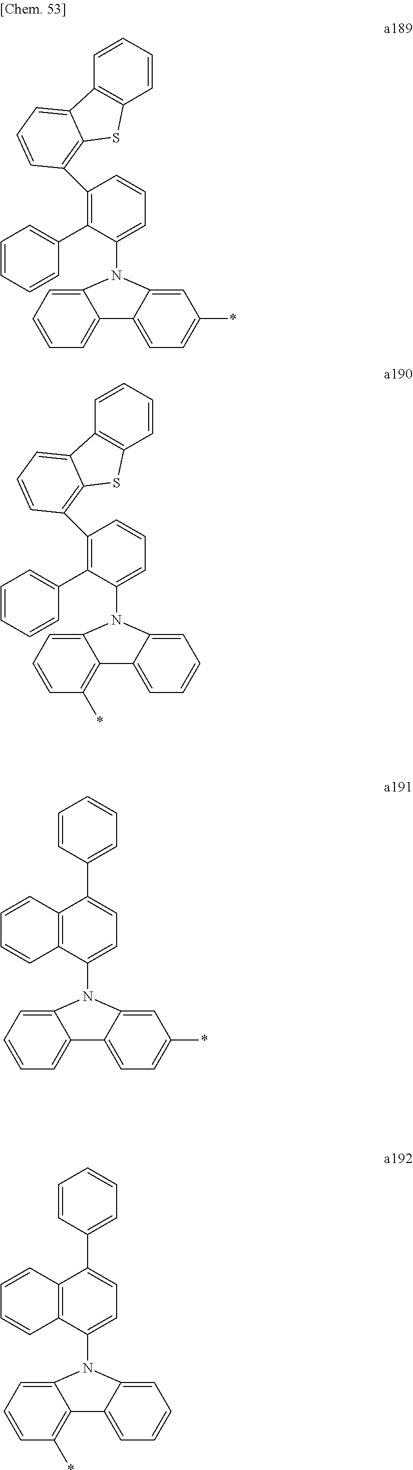

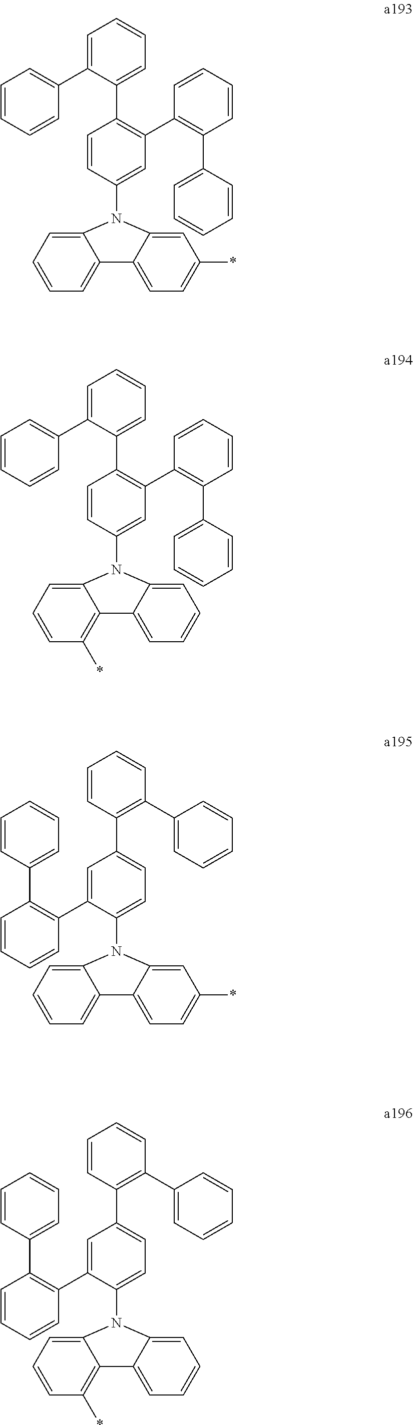 [SMILES: *c1ccc2c3ccccc3n(-c3ccc(-c4ccccc4-c4ccccc4)c(-c4ccccc4-c4ccccc4)c3)c2c1 ; *c1cccc2c1c1ccccc1n2-c1ccc(-c2ccccc2-c2ccccc2)c(-c2ccccc2-c2ccccc2)c1 ; *c1ccc2c3ccccc3n(-c3ccc(-c4ccccc4-c4ccccc4)cc3-c3ccccc3-c3ccccc3)c2c1 ; *c1cccc2c1c1ccccc1n2-c1ccc(-c2ccccc2-c2ccccc2)cc1-c1ccccc1-c1ccccc1]

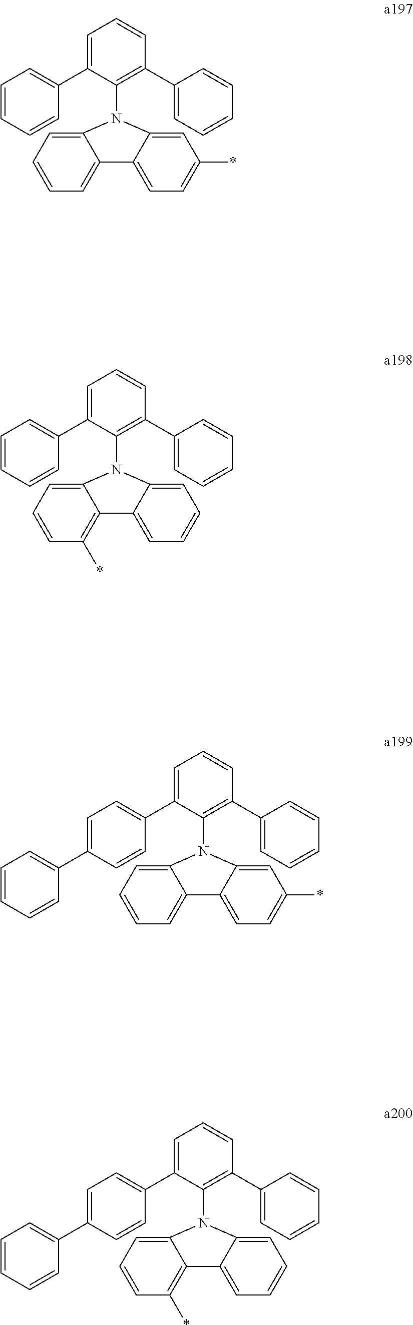 [SMILES: *c1ccc2c3ccccc3n(-c3c(-c4ccccc4)cccc3-c3ccccc3)c2c1 ; *c1cccc2c1c1ccccc1n2-c1c(-c2ccccc2)cccc1-c1ccccc1 ; *c1ccc2c3ccccc3n(-c3c(-c4ccccc4)cccc3-c3ccc(-c4ccccc4)cc3)c2c1 ; *c1cccc2c1c1ccccc1n2-c1c(-c2ccccc2)cccc1-c1ccc(-c2ccccc2)cc1]

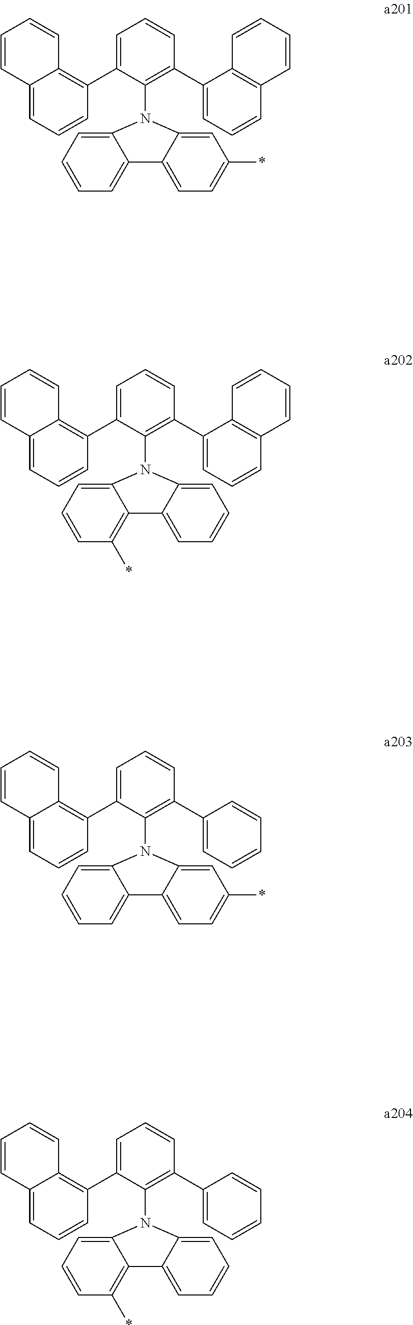 [SMILES: *c1ccc2c3ccccc3n(-c3c(-c4cccc5ccccc45)cccc3-c3cccc4ccccc34)c2c1 ; *c1cccc2c1c1ccccc1n2-c1c(-c2cccc3ccccc23)cccc1-c1cccc2ccccc12 ; *c1ccc2c3ccccc3n(-c3c(-c4ccccc4)cccc3-c3cccc4ccccc34)c2c1 ; *c1cccc2c1c1ccccc1n2-c1c(-c2ccccc2)cccc1-c1cccc2ccccc12]

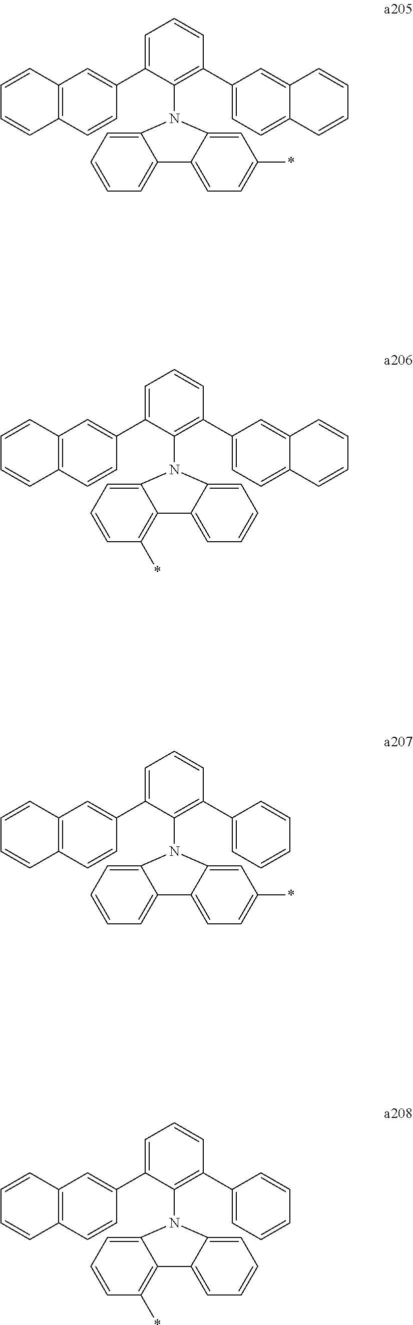 [SMILES: *c1ccc2c3ccccc3n(-c3c(-c4ccc5ccccc5c4)cccc3-c3ccc4ccccc4c3)c2c1 ; *c1cccc2c1c1ccccc1n2-c1c(-c2ccc3ccccc3c2)cccc1-c1ccc2ccccc2c1 ; *c1ccc2c3ccccc3n(-c3c(-c4ccccc4)cccc3-c3ccc4ccccc4c3)c2c1 ; *c1cccc2c1c1ccccc1n2-c1c(-c2ccccc2)cccc1-c1ccc2ccccc2c1]

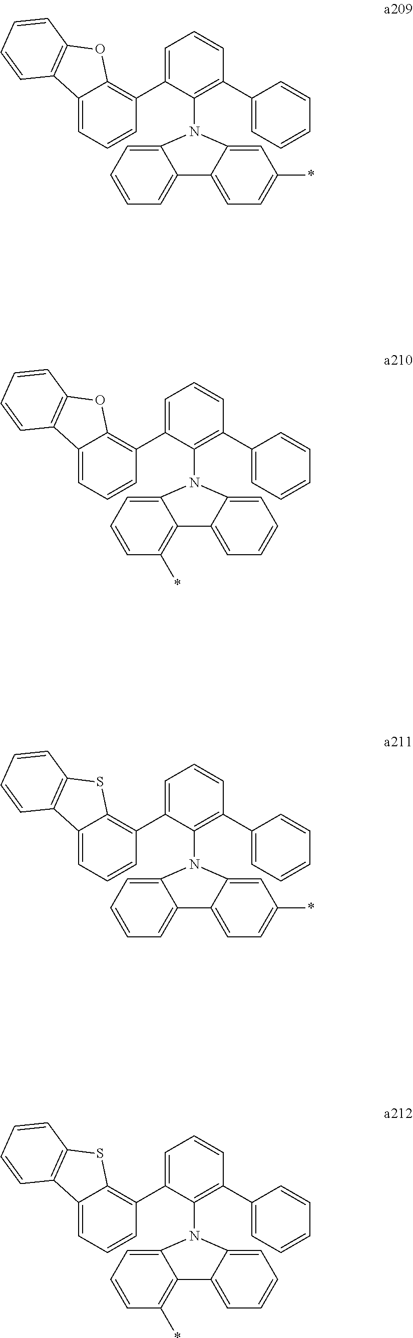 [SMILES: *c1ccc2c3ccccc3n(-c3c(-c4ccccc4)cccc3-c3cccc4c3oc3ccccc34)c2c1 ; *c1cccc2c1c1ccccc1n2-c1c(-c2ccccc2)cccc1-c1cccc2c1oc1ccccc12 ; *c1ccc2c3ccccc3n(-c3c(-c4ccccc4)cccc3-c3cccc4c3sc3ccccc34)c2c1 ; *c1cccc2c1c1ccccc1n2-c1c(-c2ccccc2)cccc1-c1cccc2c1sc1ccccc12]

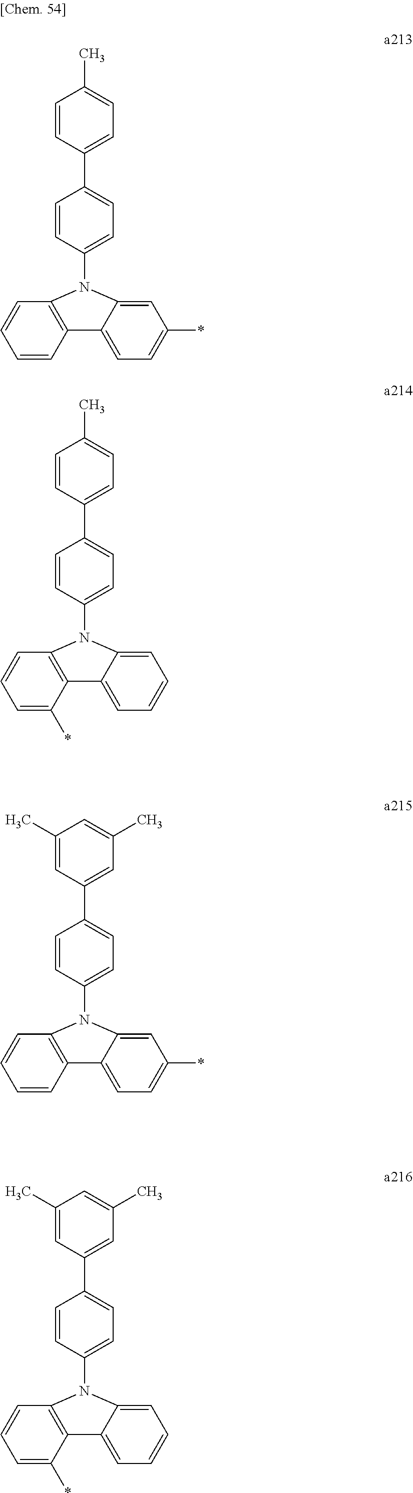 [SMILES: *c1ccc2c3ccccc3n(-c3ccc(-c4ccc(C)cc4)cc3)c2c1 ; *c1cccc2c1c1ccccc1n2-c1ccc(-c2ccc(C)cc2)cc1 ; *c1ccc2c3ccccc3n(-c3ccc(-c4cc(C)cc(C)c4)cc3)c2c1 ; *c1cccc2c1c1ccccc1n2-c1ccc(-c2cc(C)cc(C)c2)cc1]

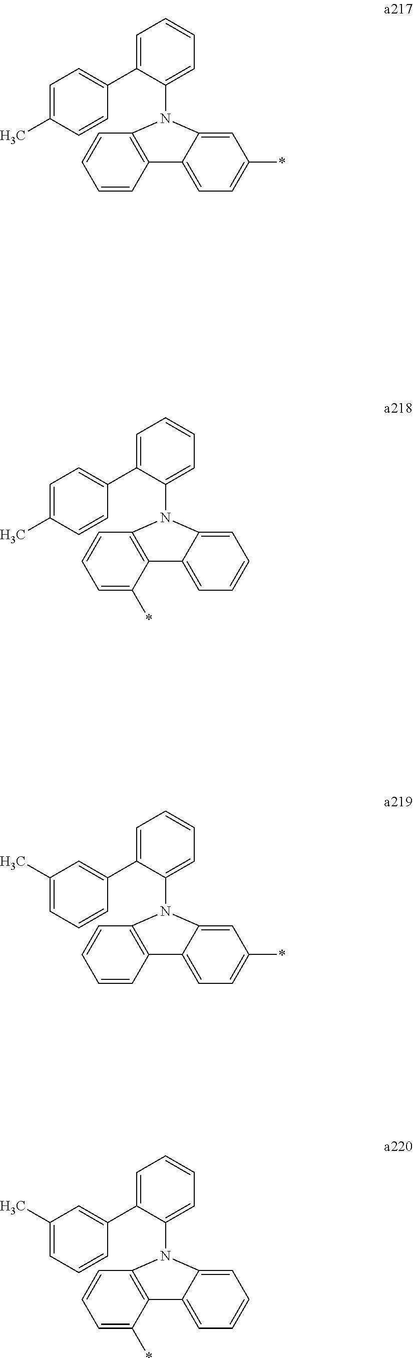 [SMILES: *c1ccc2c3ccccc3n(-c3ccccc3-c3ccc(C)cc3)c2c1 ; *c1cccc2c1c1ccccc1n2-c1ccccc1-c1ccc(C)cc1 ; *c1ccc2c3ccccc3n(-c3ccccc3-c3cccc(C)c3)c2c1 ; *c1cccc2c1c1ccccc1n2-c1ccccc1-c1cccc(C)c1]

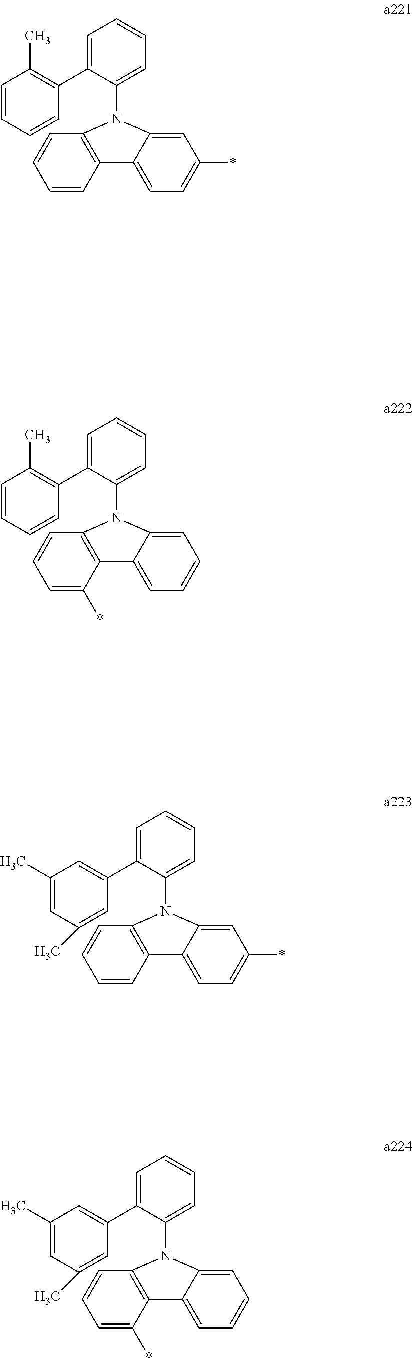 [SMILES: *c1ccc2c3ccccc3n(-c3ccccc3-c3ccccc3C)c2c1 ; *c1cccc2c1c1ccccc1n2-c1ccccc1-c1ccccc1C ; *c1ccc2c3ccccc3n(-c3ccccc3-c3cc(C)cc(C)c3)c2c1 ; *c1cccc2c1c1ccccc1n2-c1ccccc1-c1cc(C)cc(C)c1]

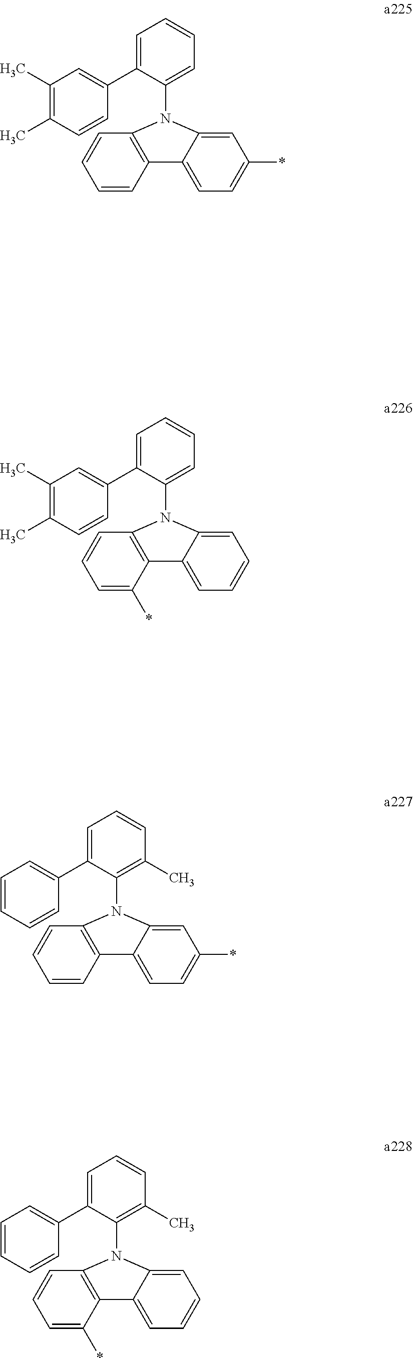 [SMILES: *c1ccc2c3ccccc3n(-c3ccccc3-c3ccc(C)c(C)c3)c2c1 ; *c1cccc2c1c1ccccc1n2-c1ccccc1-c1ccc(C)c(C)c1 ; *c1ccc2c3ccccc3n(-c3c(C)cccc3-c3ccccc3)c2c1 ; *c1cccc2c1c1ccccc1n2-c1c(C)cccc1-c1ccccc1]

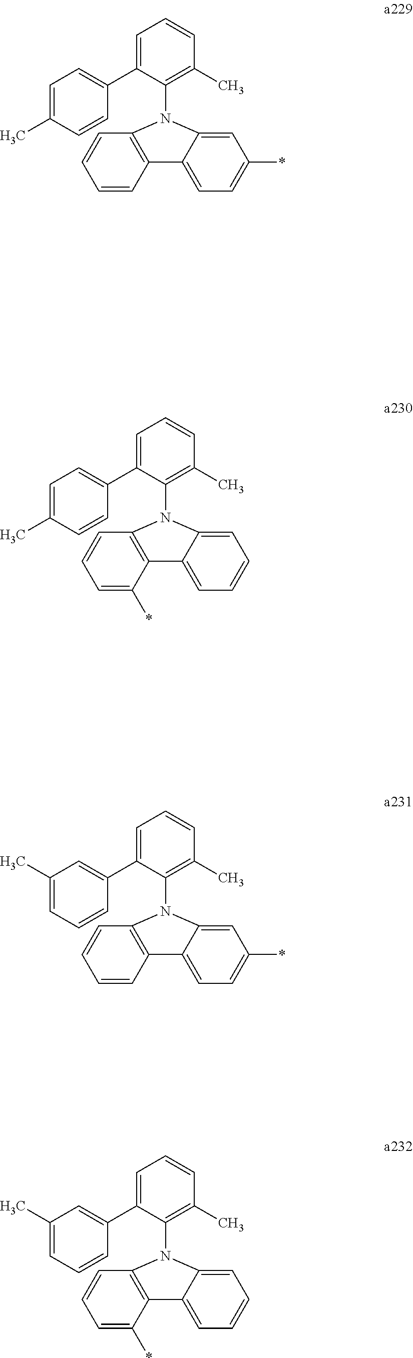 [SMILES: *c1ccc2c3ccccc3n(-c3c(C)cccc3-c3ccc(C)cc3)c2c1 ; *c1cccc2c1c1ccccc1n2-c1c(C)cccc1-c1ccc(C)cc1 ; *c1ccc2c3ccccc3n(-c3c(C)cccc3-c3cccc(C)c3)c2c1 ; *c1cccc2c1c1ccccc1n2-c1c(C)cccc1-c1cccc(C)c1]

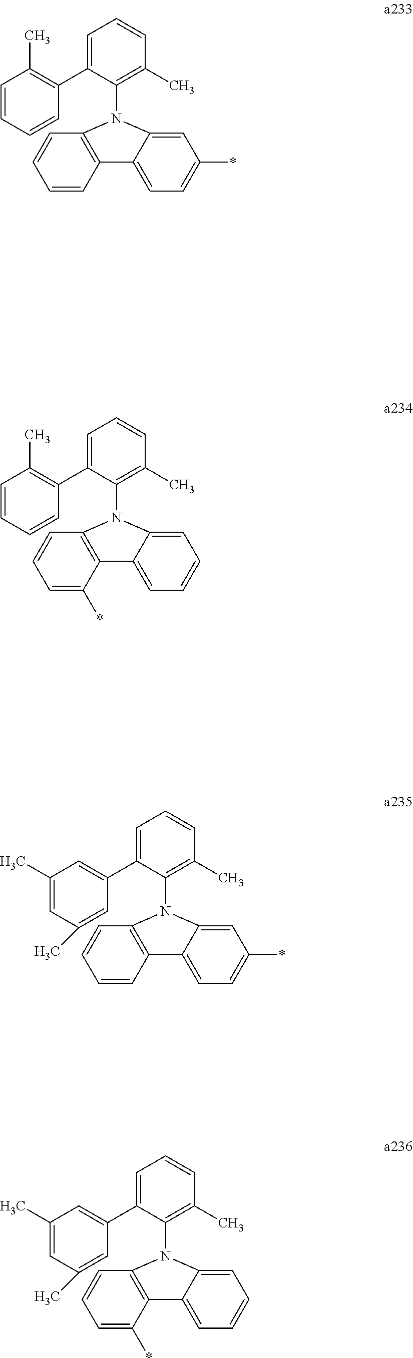 [SMILES: *c1ccc2c3ccccc3n(-c3c(C)cccc3-c3ccccc3C)c2c1 ; *c1cccc2c1c1ccccc1n2-c1c(C)cccc1-c1ccccc1C ; *c1ccc2c3ccccc3n(-c3c(C)cccc3-c3cc(C)cc(C)c3)c2c1 ; *c1cccc2c1c1ccccc1n2-c1c(C)cccc1-c1cc(C)cc(C)c1]

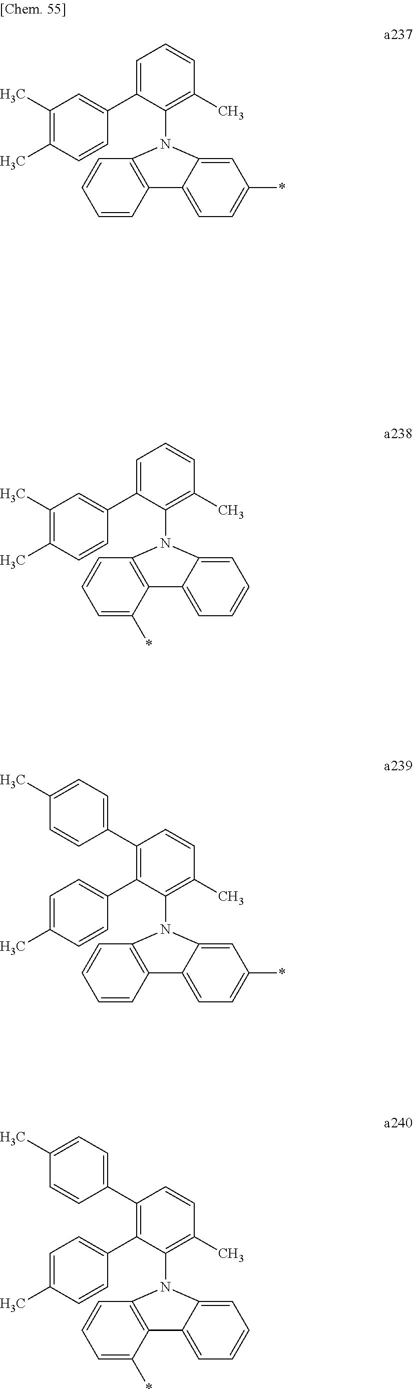 [SMILES: *c1ccc2c3ccccc3n(-c3c(C)cccc3-c3ccc(C)c(C)c3)c2c1 ; *c1cccc2c1c1ccccc1n2-c1c(C)cccc1-c1ccc(C)c(C)c1 ; *c1ccc2c3ccccc3n(-c3c(C)ccc(-c4ccc(C)cc4)c3-c3ccc(C)cc3)c2c1 ; *c1cccc2c1c1ccccc1n2-c1c(C)ccc(-c2ccc(C)cc2)c1-c1ccc(C)cc1]

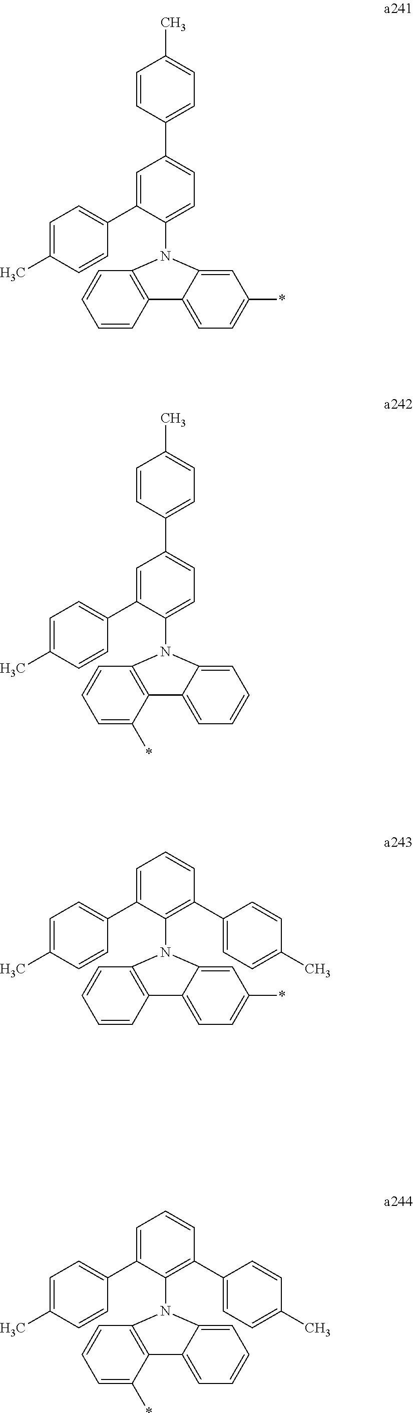

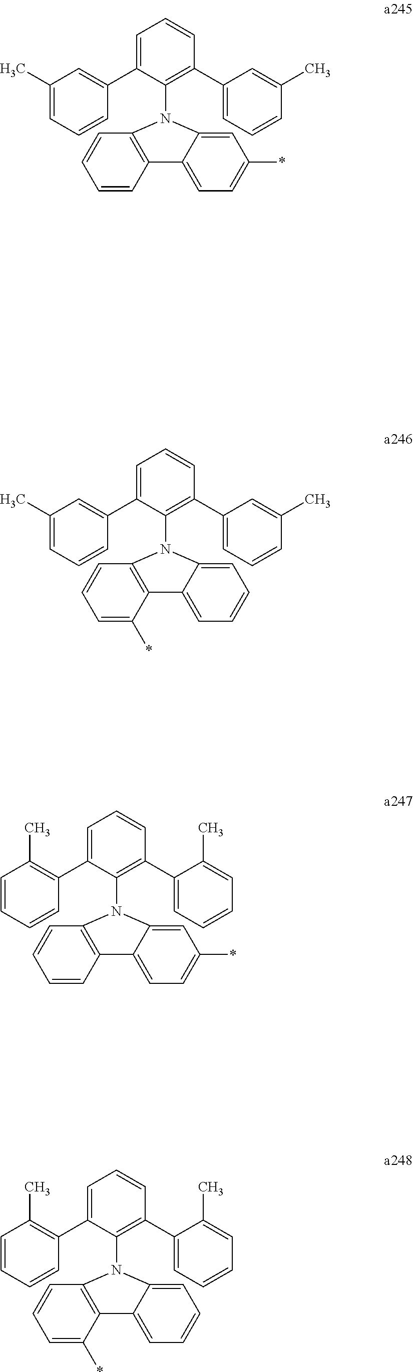

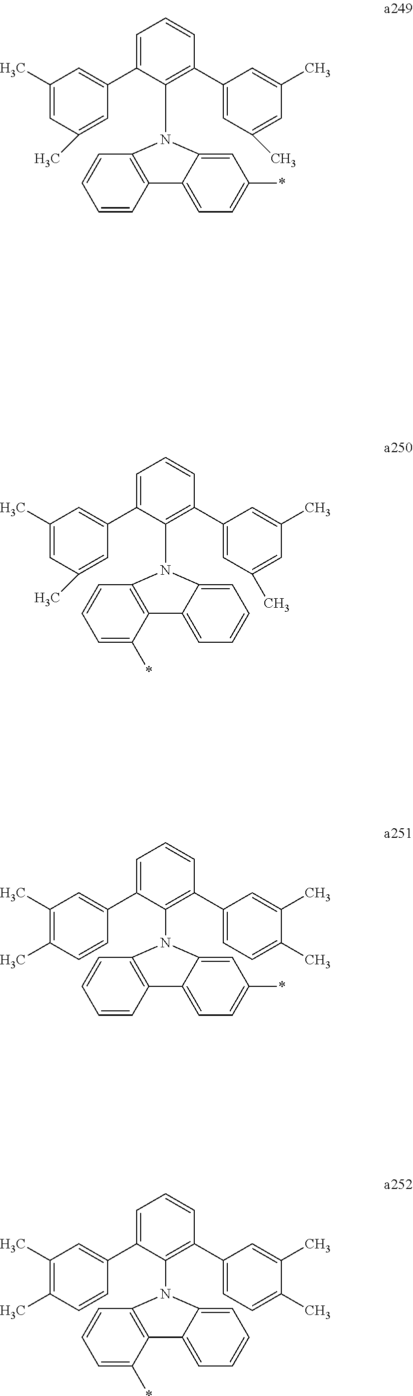

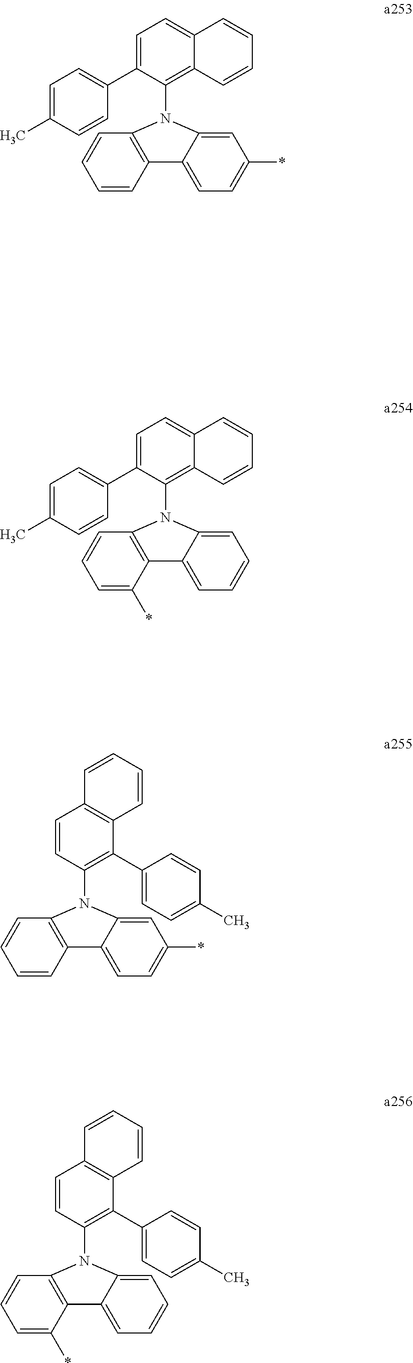 [SMILES: *c1ccc2c3ccccc3n(-c3c(-c4ccc(C)cc4)ccc4ccccc34)c2c1 ; *c1cccc2c1c1ccccc1n2-c1c(-c2ccc(C)cc2)ccc2ccccc12 ; *c1ccc2c3ccccc3n(-c3ccc4ccccc4c3-c3ccc(C)cc3)c2c1 ; *c1cccc2c1c1ccccc1n2-c1ccc2ccccc2c1-c1ccc(C)cc1]

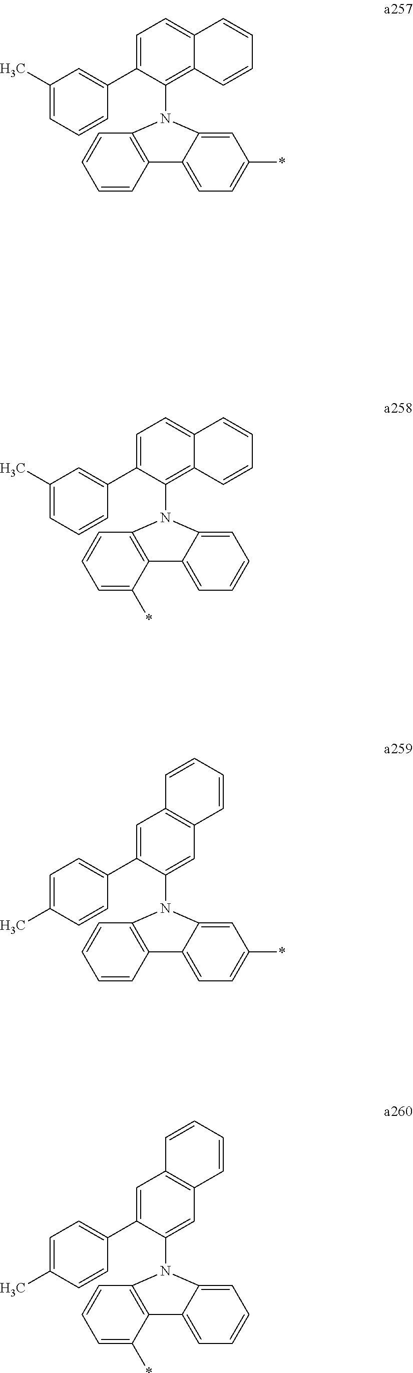 [SMILES: *c1ccc2c3ccccc3n(-c3c(-c4cccc(C)c4)ccc4ccccc34)c2c1 ; *c1cccc2c1c1ccccc1n2-c1c(-c2cccc(C)c2)ccc2ccccc12 ; *c1ccc2c3ccccc3n(-c3cc4ccccc4cc3-c3ccc(C)cc3)c2c1 ; *c1cccc2c1c1ccccc1n2-c1cc2ccccc2cc1-c1ccc(C)cc1]

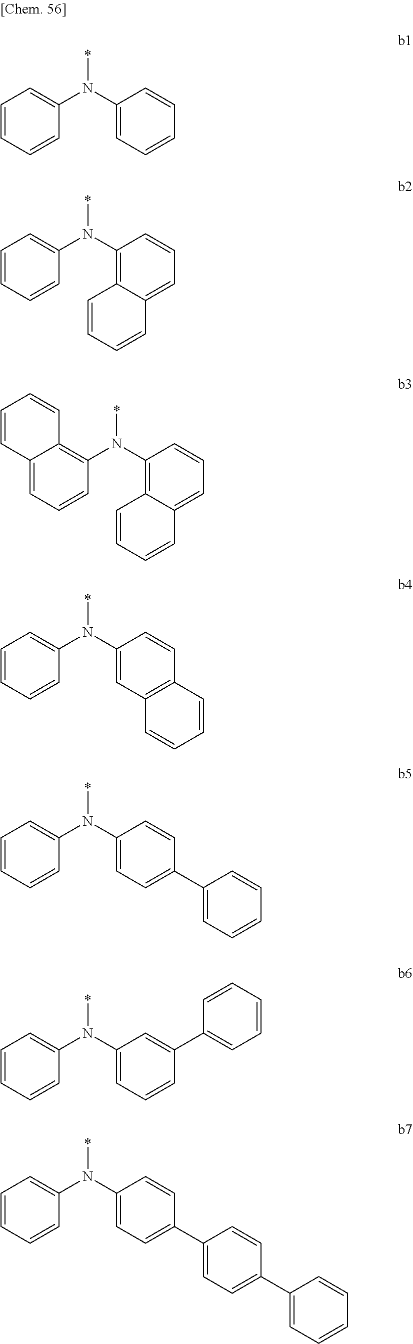

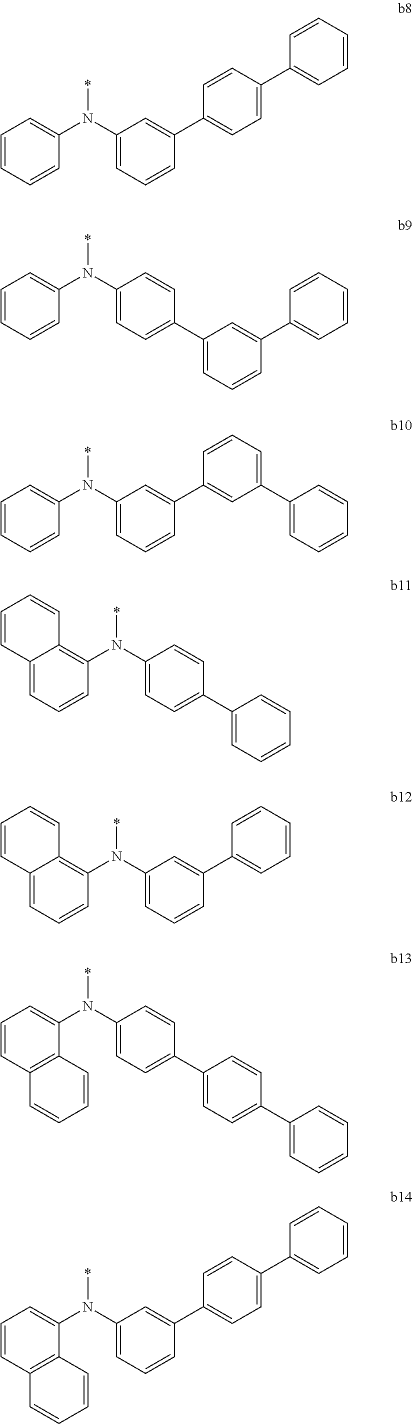

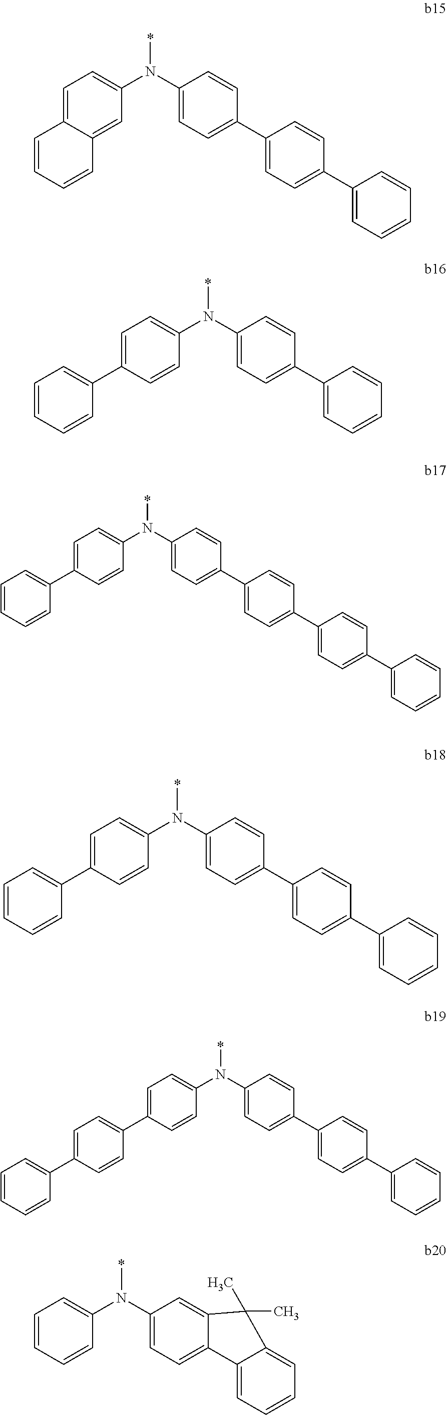 [SMILES: *N(c1ccc(-c2ccc(-c3ccccc3)cc2)cc1)c1ccc2ccccc2c1 ; *N(c1ccc(-c2ccccc2)cc1)c1ccc(-c2ccccc2)cc1 ; *N(c1ccc(-c2ccccc2)cc1)c1ccc(-c2ccc(-c3ccc(-c4ccccc4)cc3)cc2)cc1 ; *N(c1ccc(-c2ccccc2)cc1)c1ccc(-c2ccc(-c3ccccc3)cc2)cc1 ; *N(c1ccc(-c2ccc(-c3ccccc3)cc2)cc1)c1ccc(-c2ccc(-c3ccccc3)cc2)cc1 ; *N(c1ccccc1)c1ccc2c(c1)C(C)(C)c1ccccc1-2]

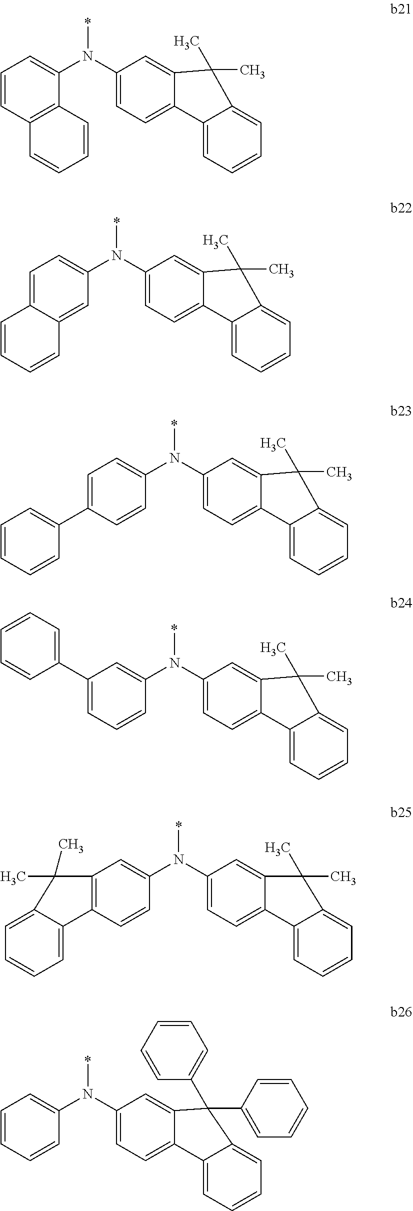

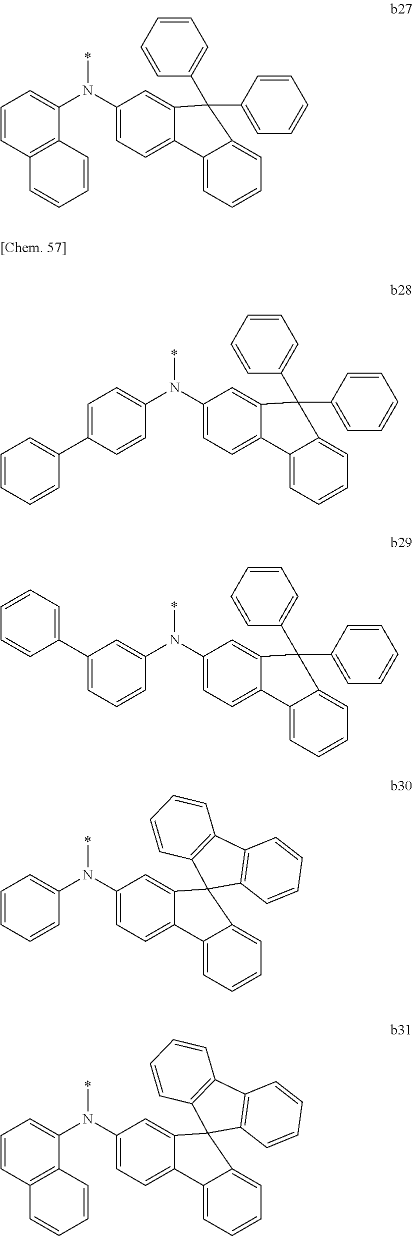 [SMILES: *N(c1ccc2c(c1)C(c1ccccc1)(c1ccccc1)c1ccccc1-2)c1cccc2ccccc12 ; *N(c1ccc(-c2ccccc2)cc1)c1ccc2c(c1)C(c1ccccc1)(c1ccccc1)c1ccccc1-2 ; *N(c1cccc(-c2ccccc2)c1)c1ccc2c(c1)C(c1ccccc1)(c1ccccc1)c1ccccc1-2 ; *N(c1ccccc1)c1ccc2c(c1)C1(c3ccccc3-c3ccccc31)c1ccccc1-2 ; *N(c1ccc2c(c1)C1(c3ccccc3-c3ccccc31)c1ccccc1-2)c1cccc2ccccc12]

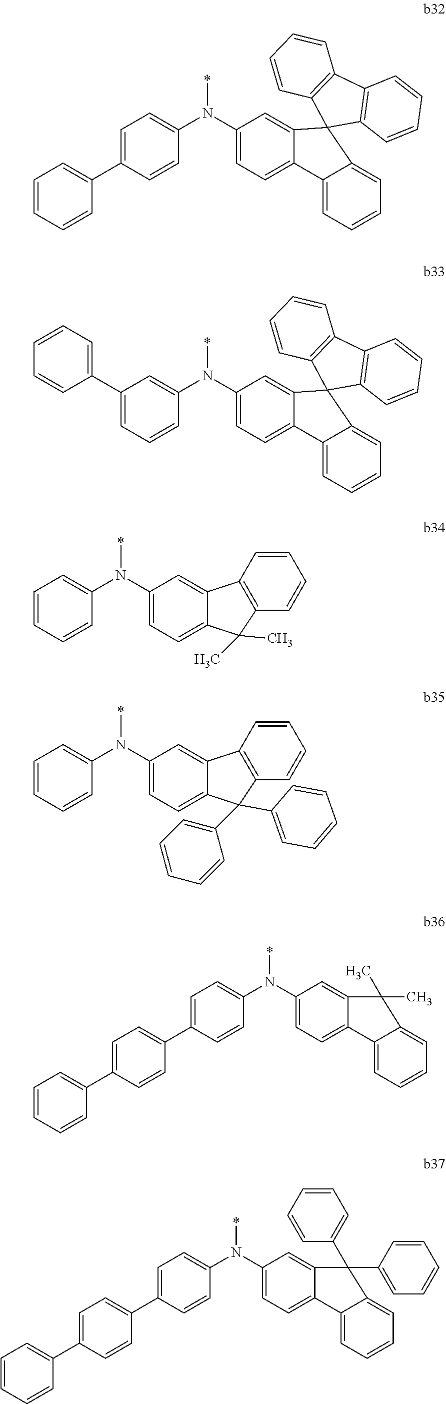 [SMILES: *N(c1ccc(-c2ccccc2)cc1)c1ccc2c(c1)C1(c3ccccc3-c3ccccc31)c1ccccc1-2 ; *N(c1cccc(-c2ccccc2)c1)c1ccc2c(c1)C1(c3ccccc3-c3ccccc31)c1ccccc1-2 ; *N(c1ccccc1)c1ccc2c(c1)-c1ccccc1C2(C)C ; *N(c1ccccc1)c1ccc2c(c1)-c1ccccc1C2(c1ccccc1)c1ccccc1 ; *N(c1ccc(-c2ccc(-c3ccccc3)cc2)cc1)c1ccc2c(c1)C(C)(C)c1ccccc1-2 ; *N(c1ccc(-c2ccc(-c3ccccc3)cc2)cc1)c1ccc2c(c1)C(c1ccccc1)(c1ccccc1)c1ccccc1-2]

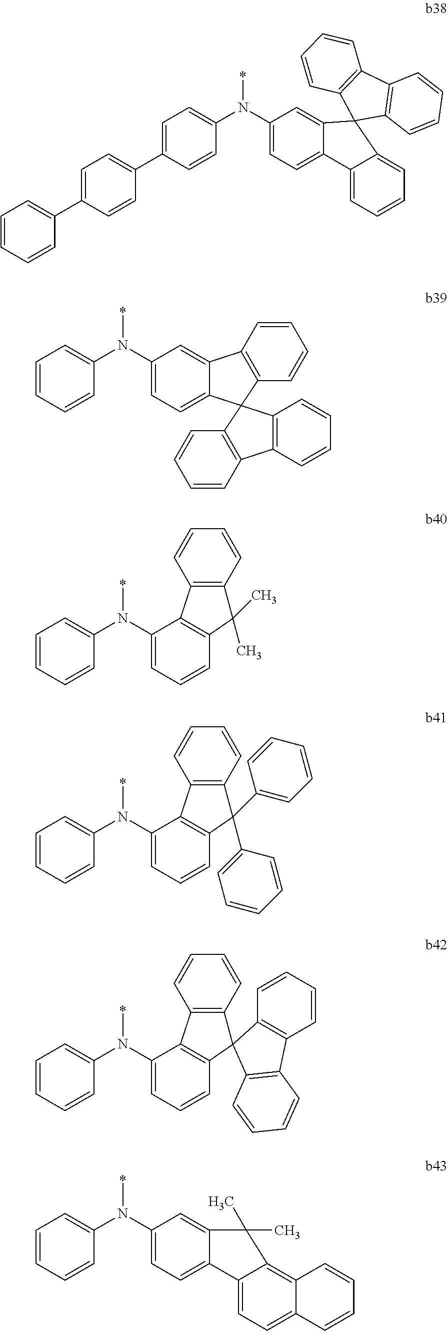

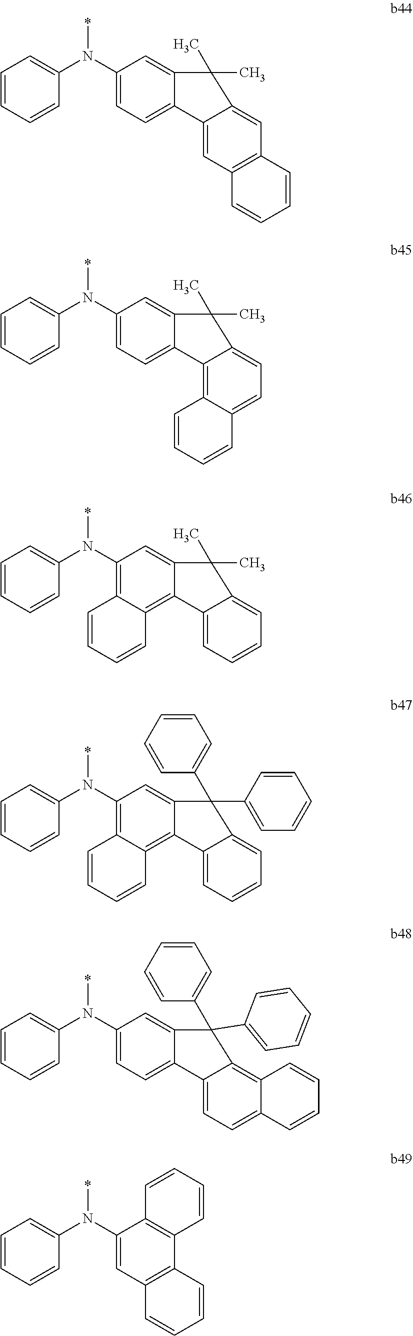 [SMILES: *N(c1ccccc1)c1ccc2c(c1)C(C)(C)c1cc3ccccc3cc1-2 ; *N(c1ccccc1)c1ccc2c(c1)C(C)(C)c1ccc3ccccc3c1-2 ; *N(c1ccccc1)c1cc2c(c3ccccc13)-c1ccccc1C2(C)C ; *N(c1ccccc1)c1cc2c(c3ccccc13)-c1ccccc1C2(c1ccccc1)c1ccccc1 ; *N(c1ccccc1)c1ccc2c(c1)C(c1ccccc1)(c1ccccc1)c1c-2ccc2ccccc12 ; *N(c1ccccc1)c1cc2ccccc2c2ccccc12]

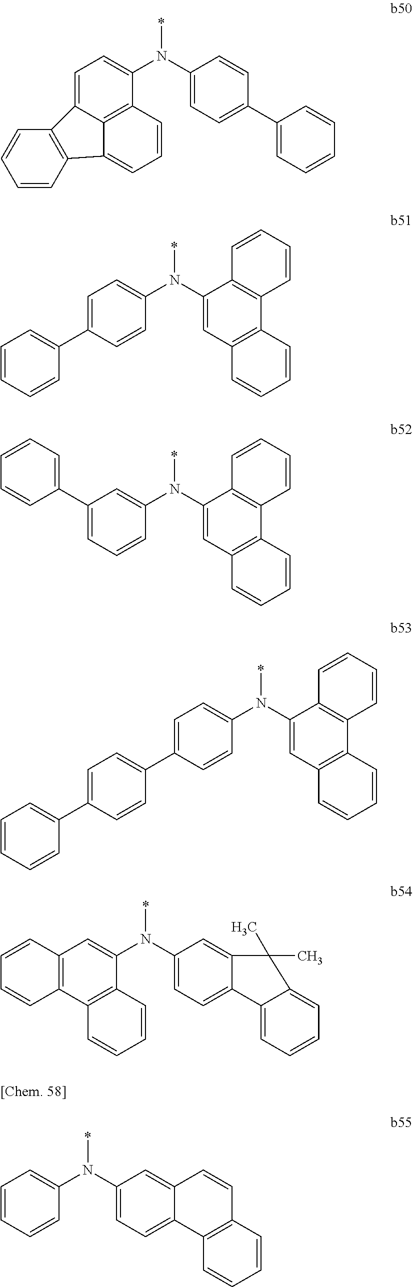

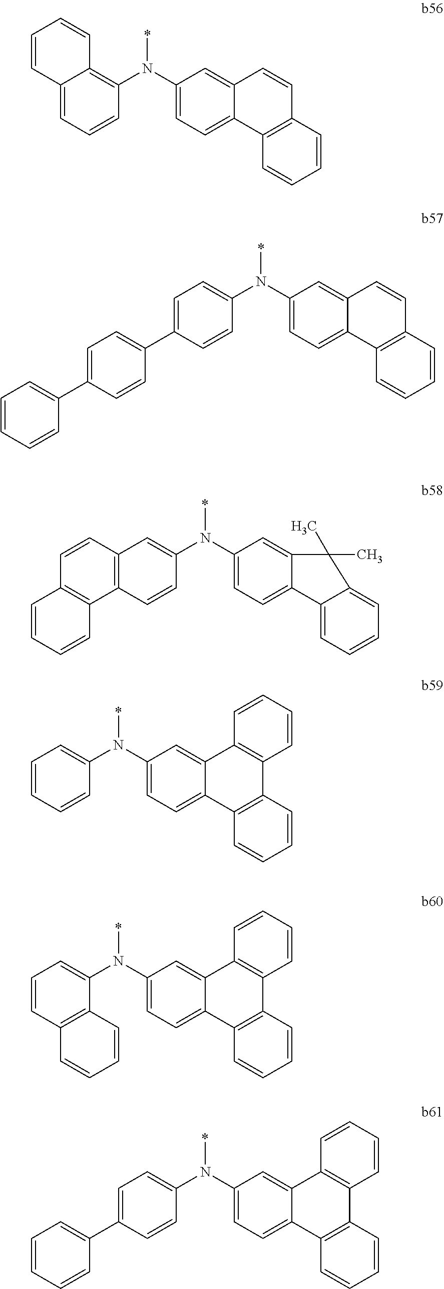 [SMILES: *N(c1ccc2c(ccc3ccccc32)c1)c1cccc2ccccc12 ; *N(c1ccc(-c2ccc(-c3ccccc3)cc2)cc1)c1ccc2c(ccc3ccccc32)c1 ; *N(c1ccc2c(c1)C(C)(C)c1ccccc1-2)c1ccc2c(ccc3ccccc32)c1 ; *N(c1ccccc1)c1ccc2c3ccccc3c3ccccc3c2c1 ; *N(c1ccc2c3ccccc3c3ccccc3c2c1)c1cccc2ccccc12 ; *N(c1ccc(-c2ccccc2)cc1)c1ccc2c3ccccc3c3ccccc3c2c1]

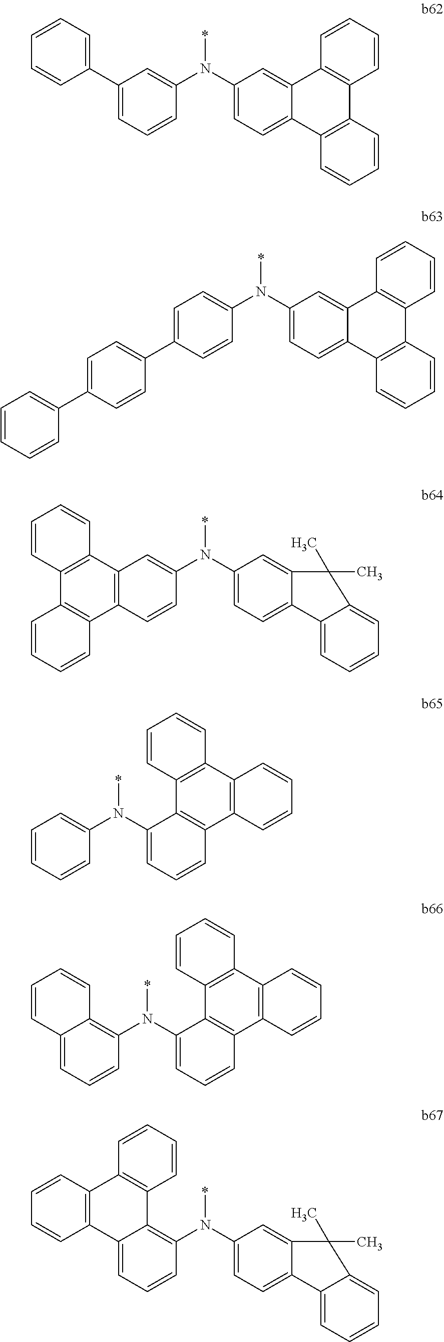 [SMILES: *N(c1cccc(-c2ccccc2)c1)c1ccc2c3ccccc3c3ccccc3c2c1 ; *N(c1ccc(-c2ccc(-c3ccccc3)cc2)cc1)c1ccc2c3ccccc3c3ccccc3c2c1 ; *N(c1ccc2c(c1)C(C)(C)c1ccccc1-2)c1ccc2c3ccccc3c3ccccc3c2c1 ; *N(c1ccccc1)c1cccc2c3ccccc3c3ccccc3c12 ; *N(c1cccc2ccccc12)c1cccc2c3ccccc3c3ccccc3c12 ; *N(c1ccc2c(c1)C(C)(C)c1ccccc1-2)c1cccc2c3ccccc3c3ccccc3c12]

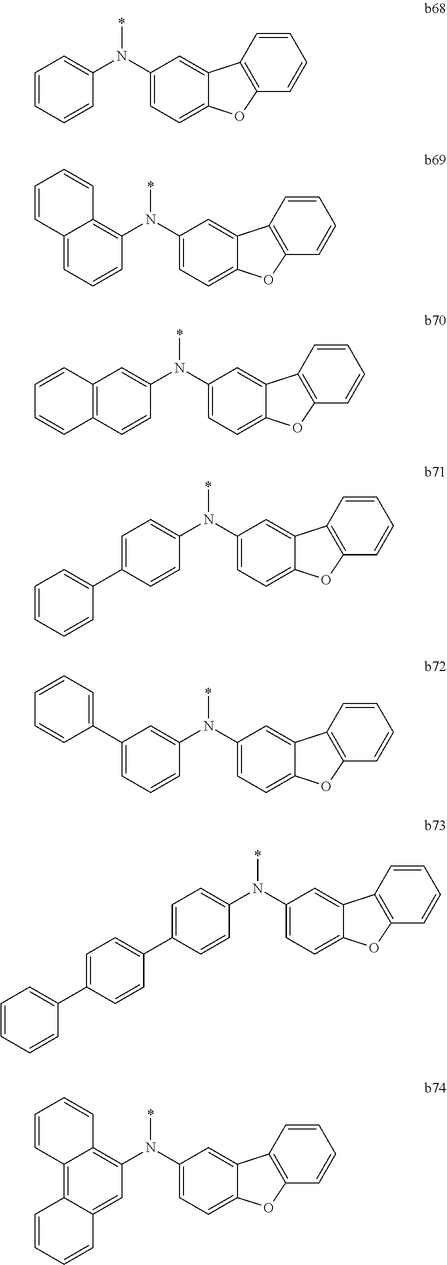

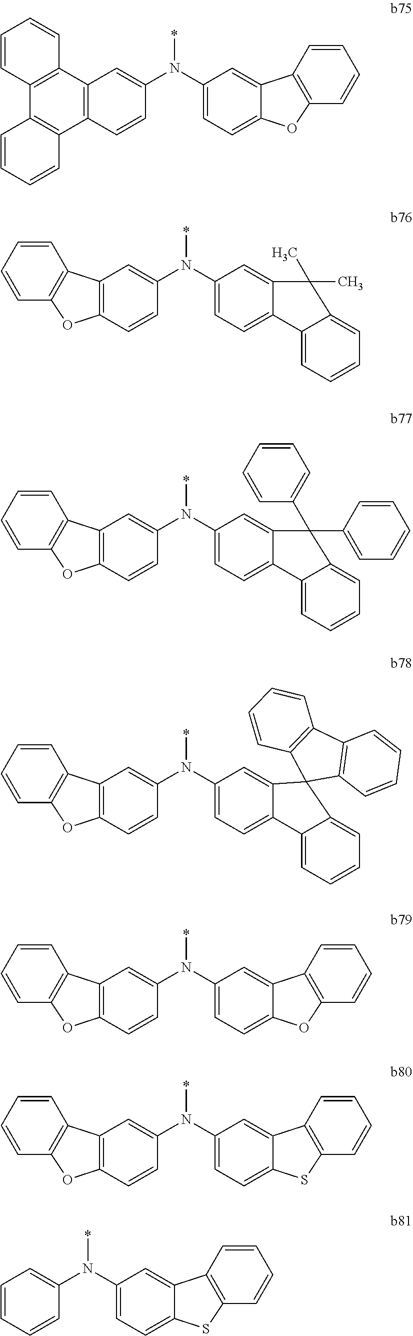

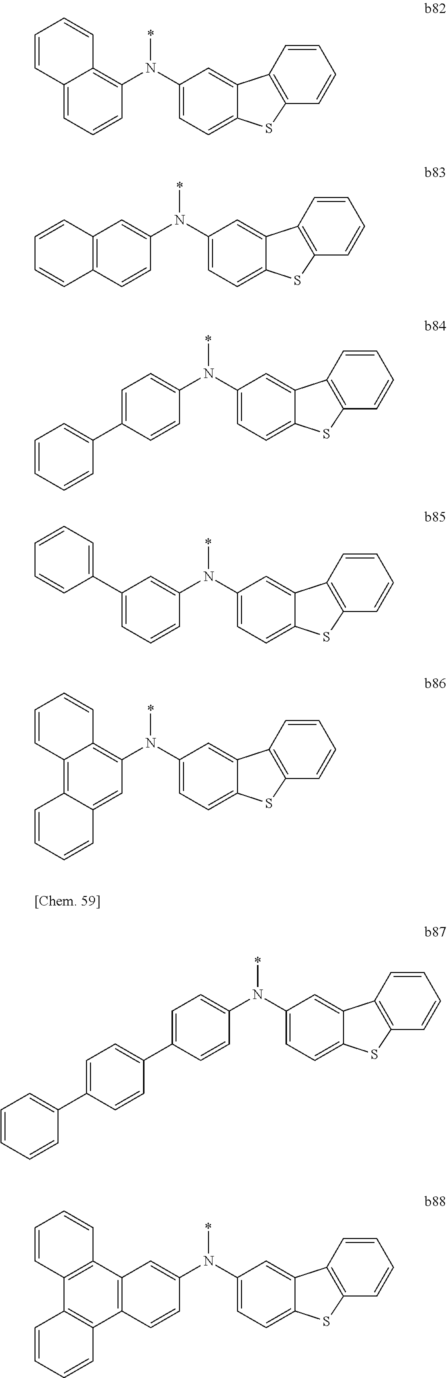

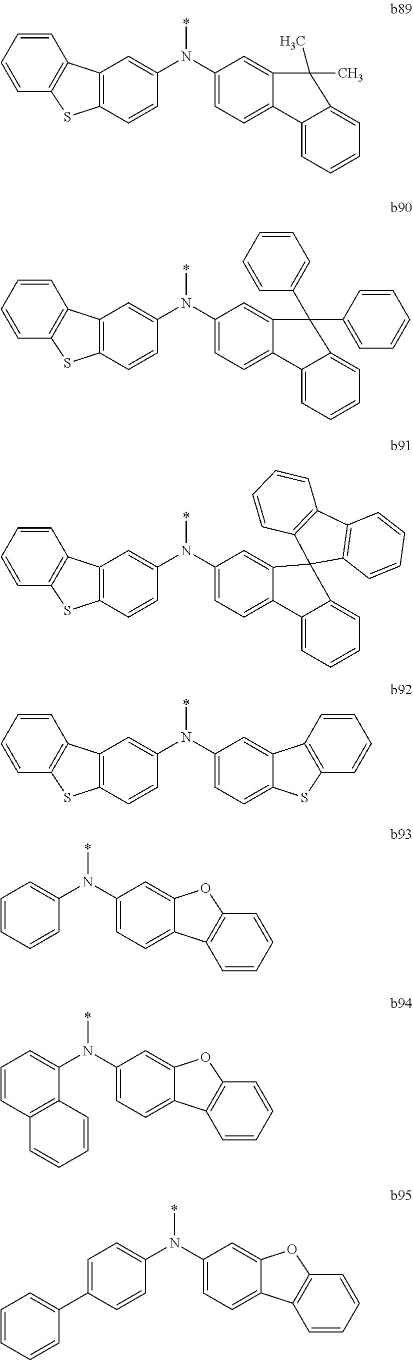

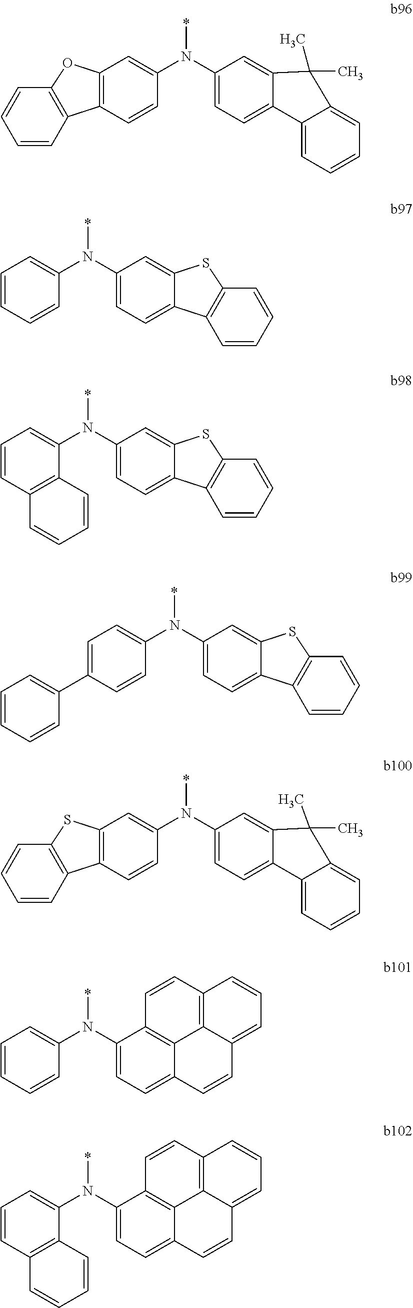

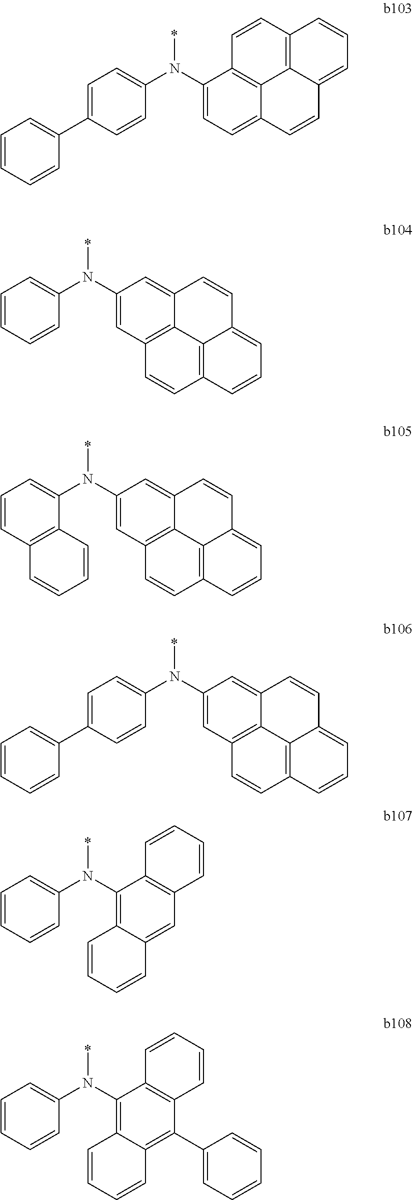 [SMILES: *N(c1ccc(-c2ccccc2)cc1)c1ccc2ccc3cccc4ccc1c2c34 ; *N(c1ccccc1)c1cc2ccc3cccc4ccc(c1)c2c34 ; *N(c1cc2ccc3cccc4ccc(c1)c2c34)c1cccc2ccccc12 ; *N(c1ccc(-c2ccccc2)cc1)c1cc2ccc3cccc4ccc(c1)c2c34 ; *N(c1ccccc1)c1c2ccccc2cc2ccccc12 ; *N(c1ccccc1)c1c2ccccc2c(-c2ccccc2)c2ccccc12]

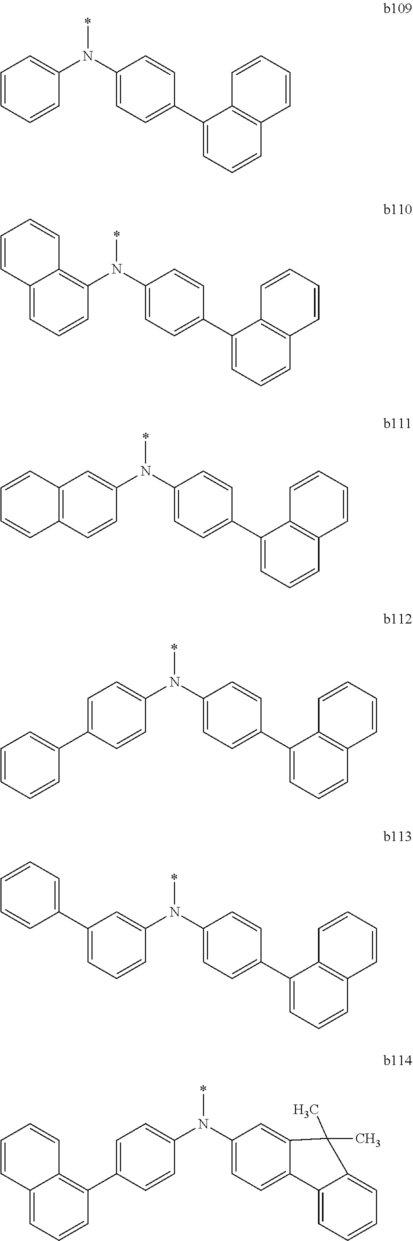 [SMILES: *N(c1ccccc1)c1ccc(-c2cccc3ccccc23)cc1 ; *N(c1ccc(-c2cccc3ccccc23)cc1)c1cccc2ccccc12 ; *N(c1ccc(-c2cccc3ccccc23)cc1)c1ccc2ccccc2c1 ; *N(c1ccc(-c2ccccc2)cc1)c1ccc(-c2cccc3ccccc23)cc1 ; *N(c1ccc(-c2cccc3ccccc23)cc1)c1cccc(-c2ccccc2)c1 ; *N(c1ccc(-c2cccc3ccccc23)cc1)c1ccc2c(c1)C(C)(C)c1ccccc1-2]

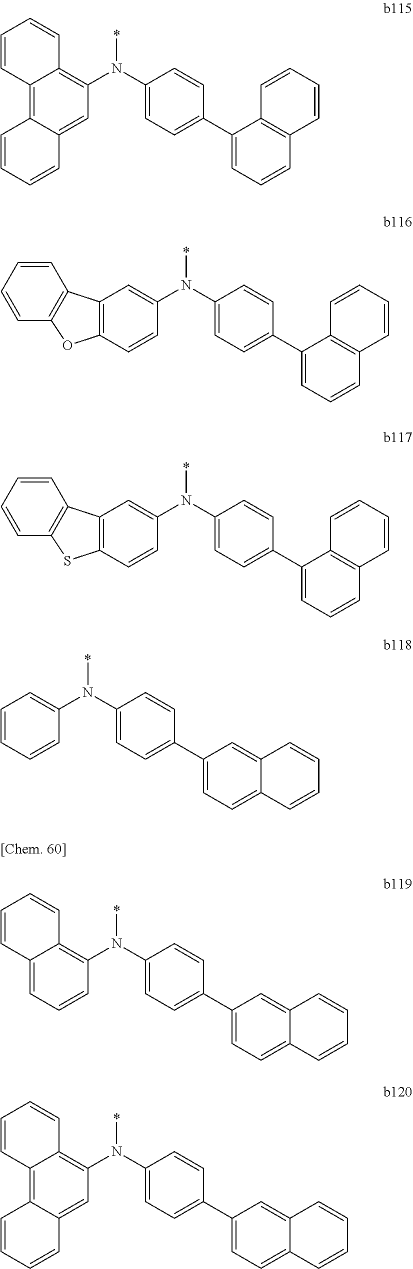 [SMILES: *N(c1ccc(-c2cccc3ccccc23)cc1)c1cc2ccccc2c2ccccc12 ; *N(c1ccc(-c2cccc3ccccc23)cc1)c1ccc2oc3ccccc3c2c1 ; *N(c1ccc(-c2cccc3ccccc23)cc1)c1ccc2sc3ccccc3c2c1 ; *N(c1ccccc1)c1ccc(-c2ccc3ccccc3c2)cc1 ; *N(c1ccc(-c2ccc3ccccc3c2)cc1)c1cccc2ccccc12 ; *N(c1ccc(-c2ccc3ccccc3c2)cc1)c1cc2ccccc2c2ccccc12]

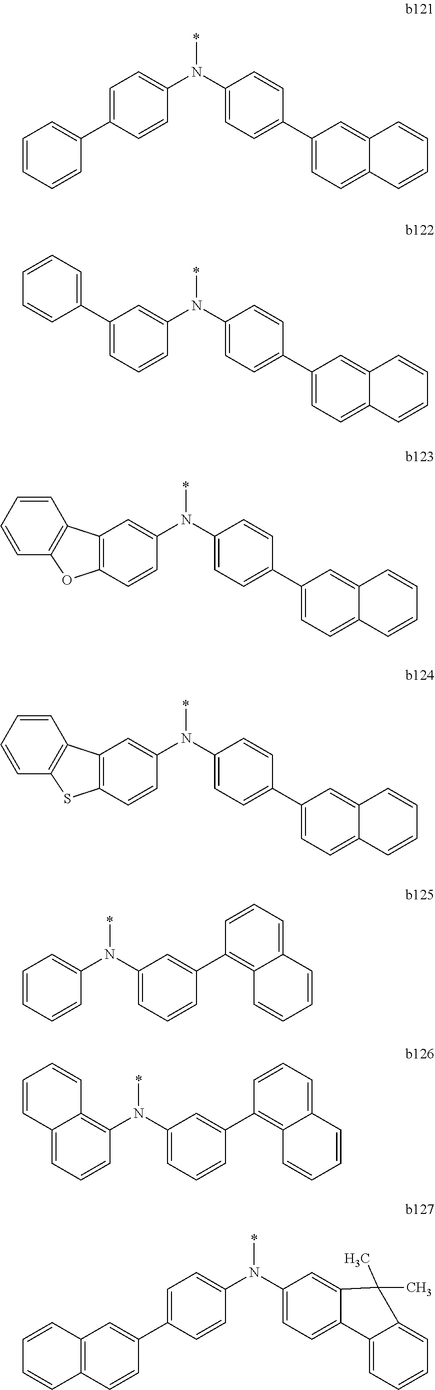

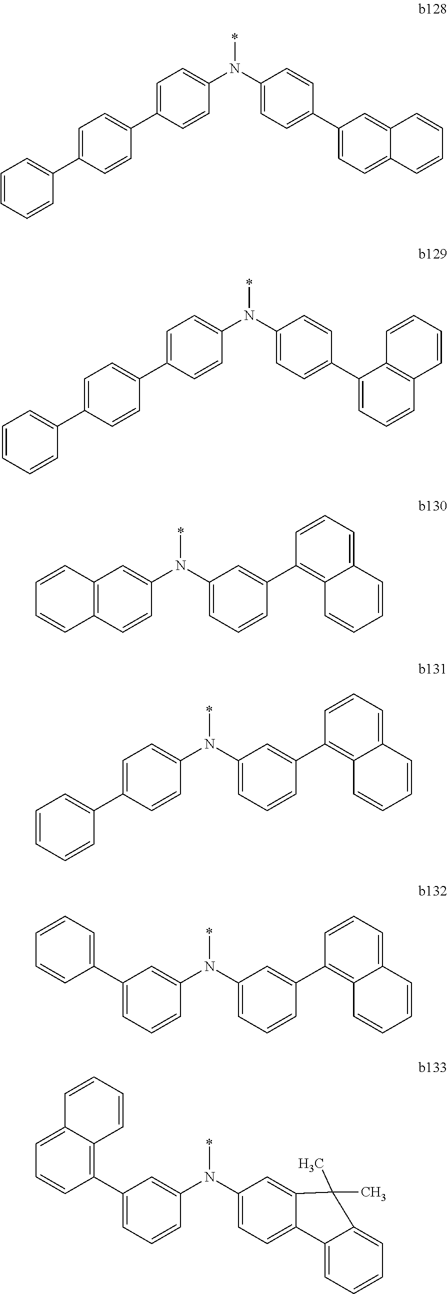 [SMILES: *N(c1ccc(-c2ccc(-c3ccccc3)cc2)cc1)c1ccc(-c2ccc3ccccc3c2)cc1 ; *N(c1ccc(-c2ccc(-c3ccccc3)cc2)cc1)c1ccc(-c2cccc3ccccc23)cc1 ; *N(c1cccc(-c2cccc3ccccc23)c1)c1ccc2ccccc2c1 ; *N(c1ccc(-c2ccccc2)cc1)c1cccc(-c2cccc3ccccc23)c1 ; *N(c1cccc(-c2ccccc2)c1)c1cccc(-c2cccc3ccccc23)c1 ; *N(c1cccc(-c2cccc3ccccc23)c1)c1ccc2c(c1)C(C)(C)c1ccccc1-2]

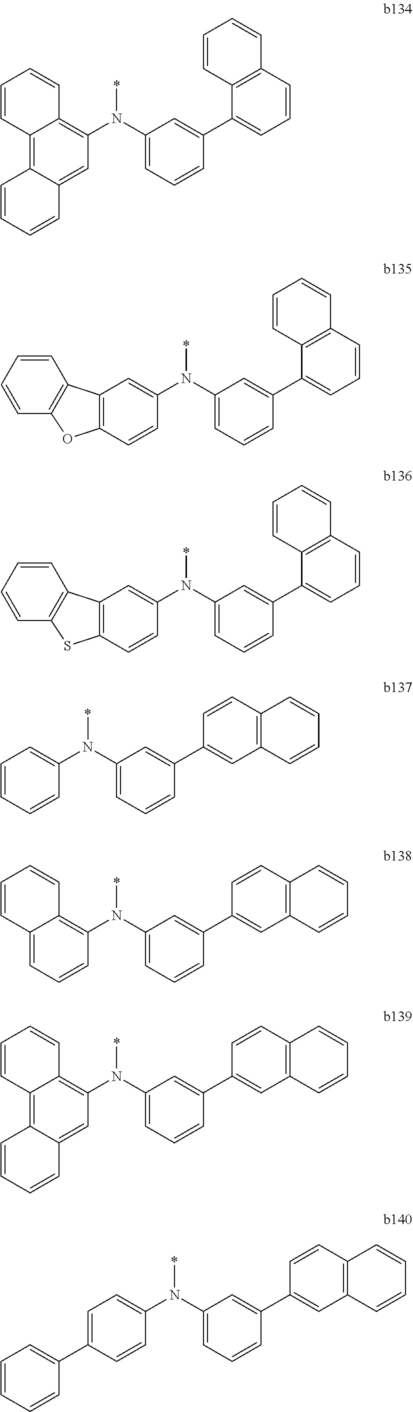

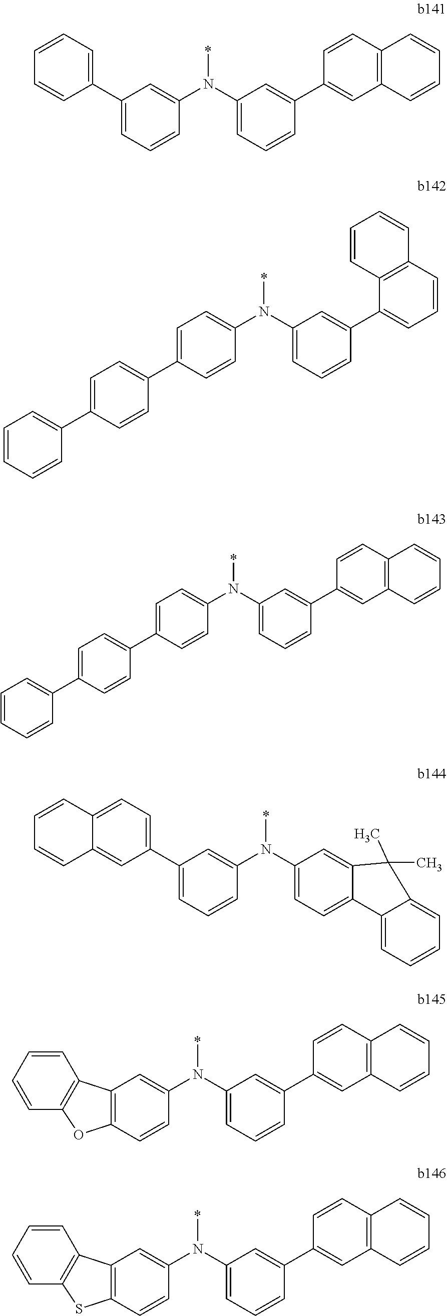 [SMILES: *N(c1cccc(-c2ccccc2)c1)c1cccc(-c2ccc3ccccc3c2)c1 ; *N(c1ccc(-c2ccc(-c3ccccc3)cc2)cc1)c1cccc(-c2cccc3ccccc23)c1 ; *N(c1ccc(-c2ccc(-c3ccccc3)cc2)cc1)c1cccc(-c2ccc3ccccc3c2)c1 ; *N(c1cccc(-c2ccc3ccccc3c2)c1)c1ccc2c(c1)C(C)(C)c1ccccc1-2 ; *N(c1cccc(-c2ccc3ccccc3c2)c1)c1ccc2oc3ccccc3c2c1 ; *N(c1cccc(-c2ccc3ccccc3c2)c1)c1ccc2sc3ccccc3c2c1]

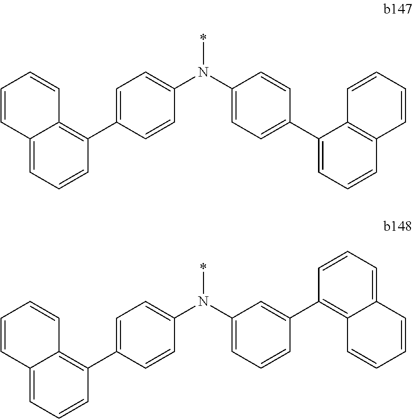 [SMILES: *N(c1ccc(-c2cccc3ccccc23)cc1)c1ccc(-c2cccc3ccccc23)cc1 ; *N(c1ccc(-c2cccc3ccccc23)cc1)c1cccc(-c2cccc3ccccc23)c1]

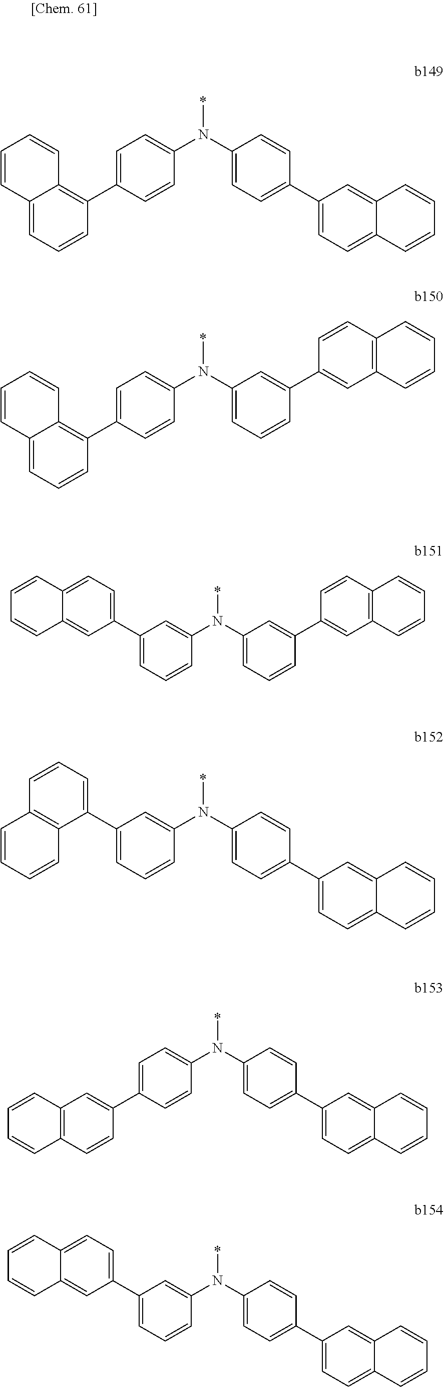 [SMILES: *N(c1ccc(-c2ccc3ccccc3c2)cc1)c1ccc(-c2cccc3ccccc23)cc1 ; *N(c1ccc(-c2cccc3ccccc23)cc1)c1cccc(-c2ccc3ccccc3c2)c1 ; *N(c1cccc(-c2ccc3ccccc3c2)c1)c1cccc(-c2ccc3ccccc3c2)c1 ; *N(c1ccc(-c2ccc3ccccc3c2)cc1)c1cccc(-c2cccc3ccccc23)c1 ; *N(c1ccc(-c2ccc3ccccc3c2)cc1)c1ccc(-c2ccc3ccccc3c2)cc1 ; *N(c1ccc(-c2ccc3ccccc3c2)cc1)c1cccc(-c2ccc3ccccc3c2)c1]

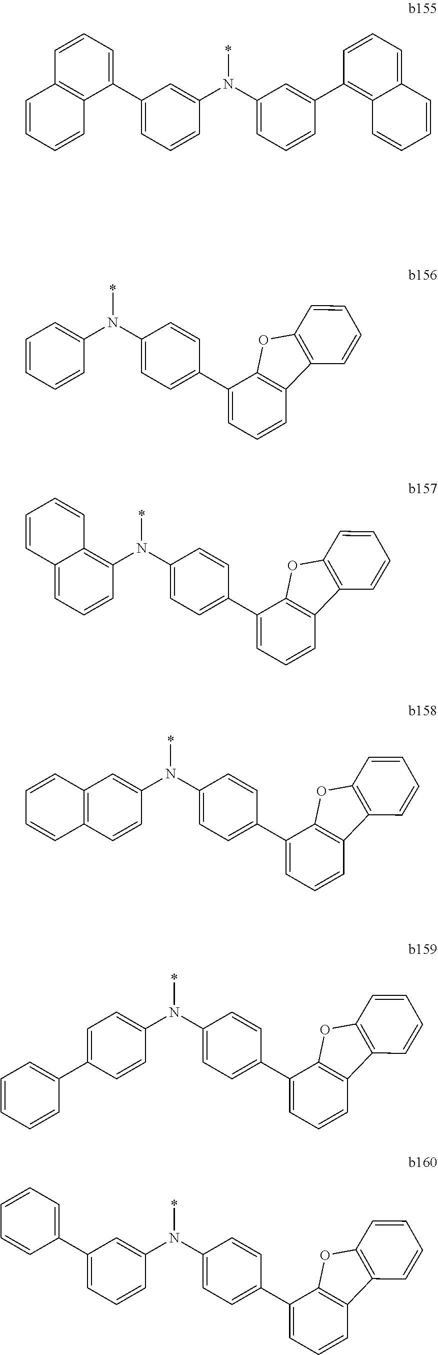 [SMILES: *N(c1cccc(-c2cccc3ccccc23)c1)c1cccc(-c2cccc3ccccc23)c1 ; *N(c1ccccc1)c1ccc(-c2cccc3c2oc2ccccc23)cc1 ; *N(c1ccc(-c2cccc3c2oc2ccccc23)cc1)c1cccc2ccccc12 ; *N(c1ccc(-c2cccc3c2oc2ccccc23)cc1)c1ccc2ccccc2c1 ; *N(c1ccc(-c2ccccc2)cc1)c1ccc(-c2cccc3c2oc2ccccc23)cc1 ; *N(c1ccc(-c2cccc3c2oc2ccccc23)cc1)c1cccc(-c2ccccc2)c1]

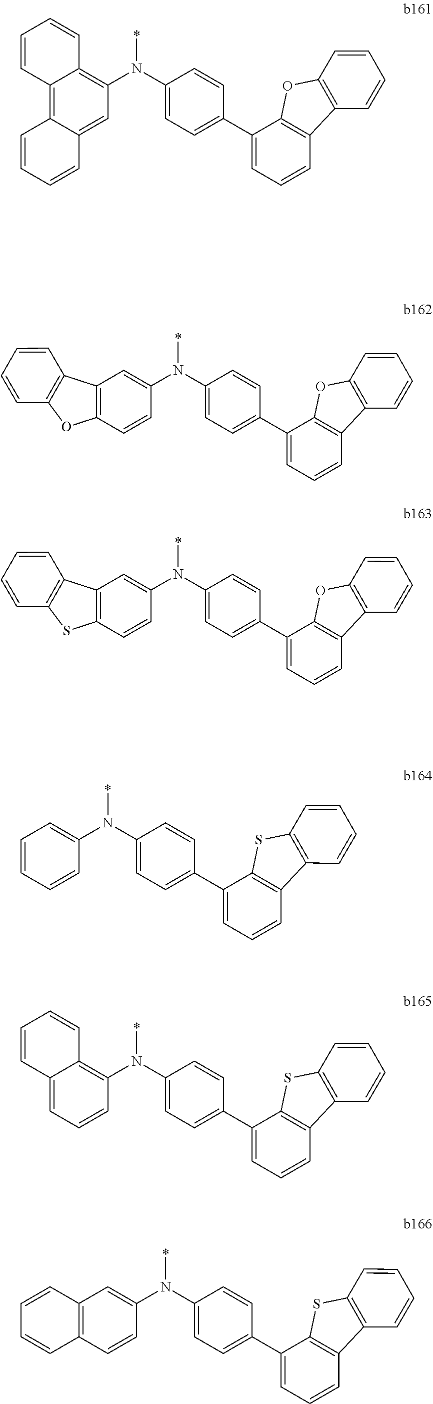 [SMILES: *N(c1ccc(-c2cccc3c2oc2ccccc23)cc1)c1cc2ccccc2c2ccccc12 ; *N(c1ccc(-c2cccc3c2oc2ccccc23)cc1)c1ccc2oc3ccccc3c2c1 ; *N(c1ccc(-c2cccc3c2oc2ccccc23)cc1)c1ccc2sc3ccccc3c2c1 ; *N(c1ccccc1)c1ccc(-c2cccc3c2sc2ccccc23)cc1 ; *N(c1ccc(-c2cccc3c2sc2ccccc23)cc1)c1cccc2ccccc12 ; *N(c1ccc(-c2cccc3c2sc2ccccc23)cc1)c1ccc2ccccc2c1]

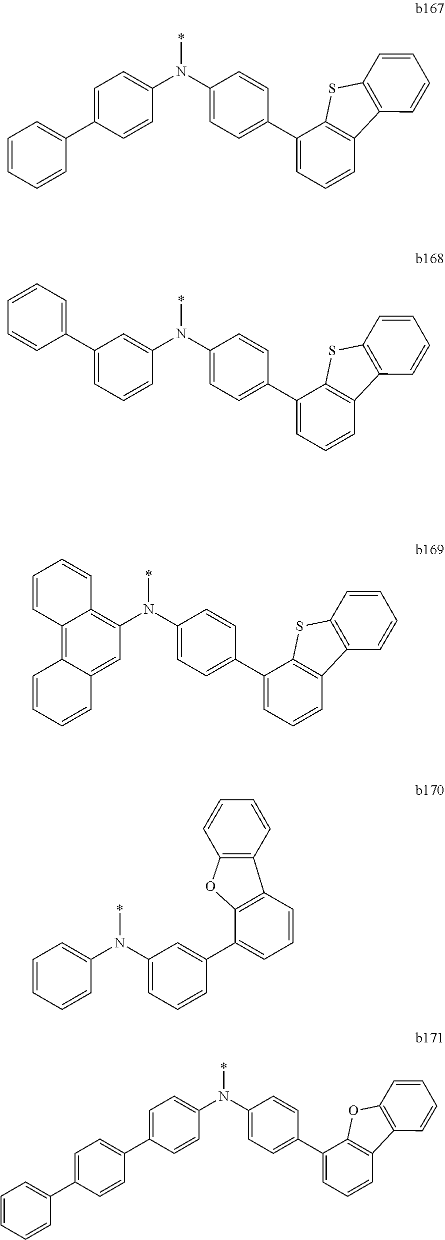 [SMILES: *N(c1ccc(-c2ccccc2)cc1)c1ccc(-c2cccc3c2sc2ccccc23)cc1 ; *N(c1ccc(-c2cccc3c2sc2ccccc23)cc1)c1cccc(-c2ccccc2)c1 ; *N(c1ccc(-c2cccc3c2sc2ccccc23)cc1)c1cc2ccccc2c2ccccc12 ; *N(c1ccccc1)c1cccc(-c2cccc3c2oc2ccccc23)c1 ; *N(c1ccc(-c2ccc(-c3ccccc3)cc2)cc1)c1ccc(-c2cccc3c2oc2ccccc23)cc1]

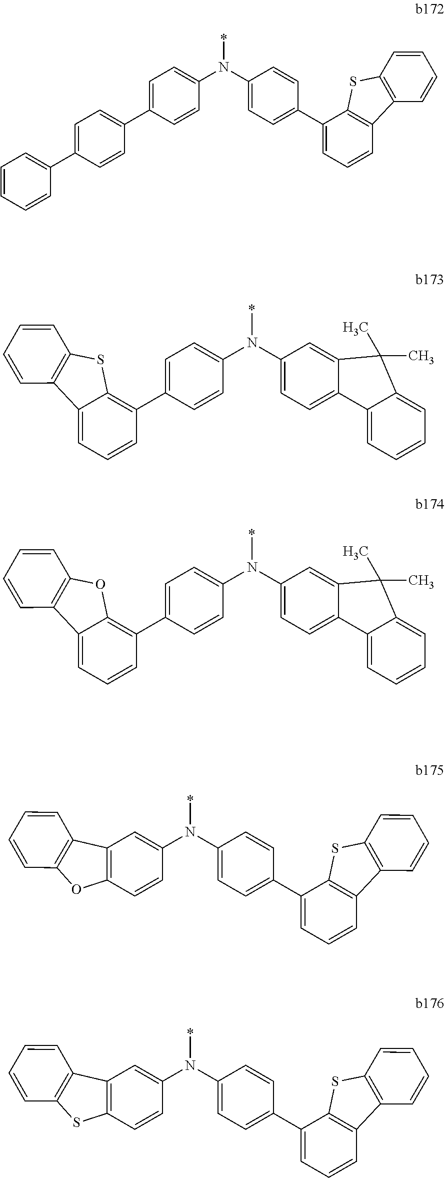 [SMILES: *N(c1ccc(-c2ccc(-c3ccccc3)cc2)cc1)c1ccc(-c2cccc3c2sc2ccccc23)cc1 ; *N(c1ccc(-c2cccc3c2sc2ccccc23)cc1)c1ccc2c(c1)C(C)(C)c1ccccc1-2 ; *N(c1ccc(-c2cccc3c2oc2ccccc23)cc1)c1ccc2c(c1)C(C)(C)c1ccccc1-2 ; *N(c1ccc(-c2cccc3c2sc2ccccc23)cc1)c1ccc2oc3ccccc3c2c1 ; *N(c1ccc(-c2cccc3c2sc2ccccc23)cc1)c1ccc2sc3ccccc3c2c1]

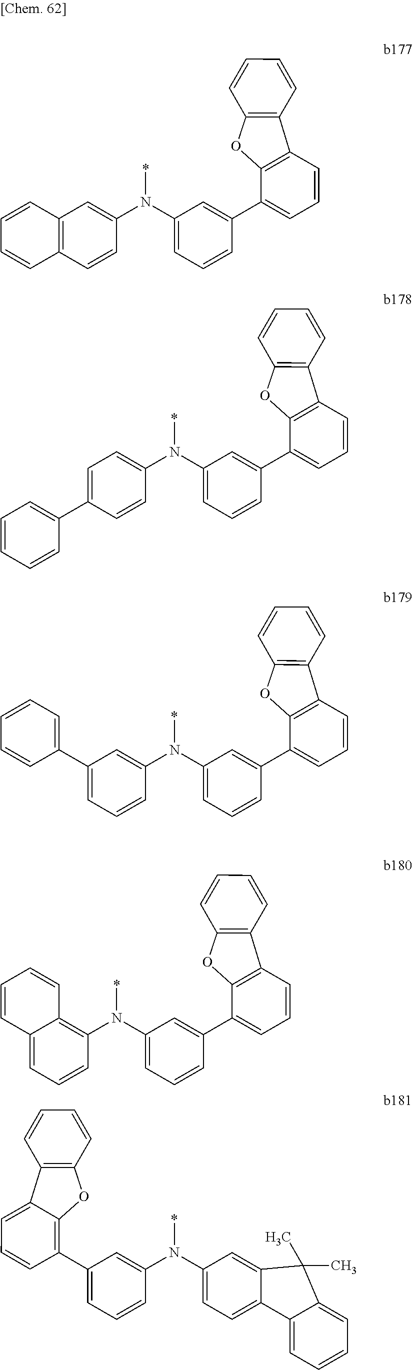

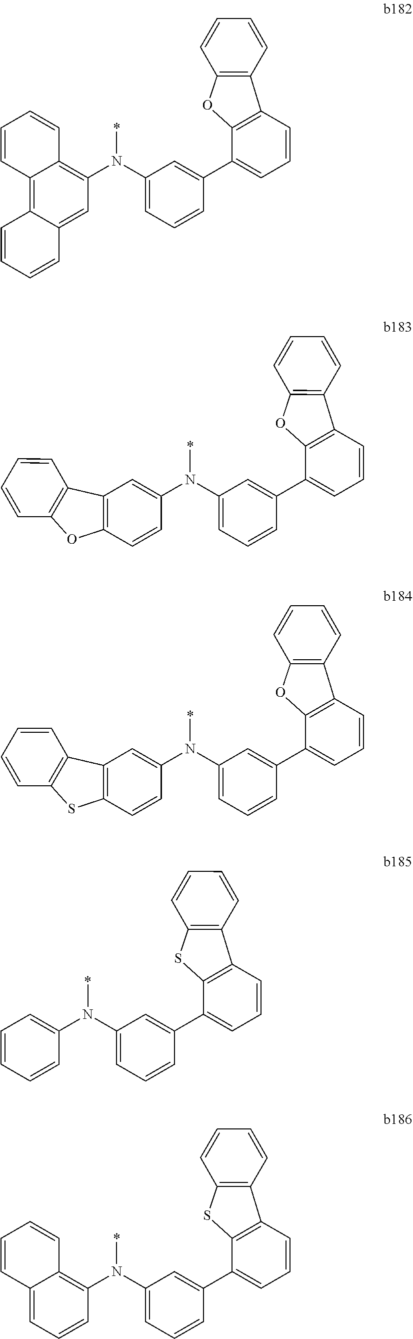 [SMILES: *N(c1cccc(-c2cccc3c2oc2ccccc23)c1)c1cc2ccccc2c2ccccc12 ; *N(c1cccc(-c2cccc3c2oc2ccccc23)c1)c1ccc2oc3ccccc3c2c1 ; *N(c1cccc(-c2cccc3c2oc2ccccc23)c1)c1ccc2sc3ccccc3c2c1 ; *N(c1ccccc1)c1cccc(-c2cccc3c2sc2ccccc23)c1 ; *N(c1cccc(-c2cccc3c2sc2ccccc23)c1)c1cccc2ccccc12]

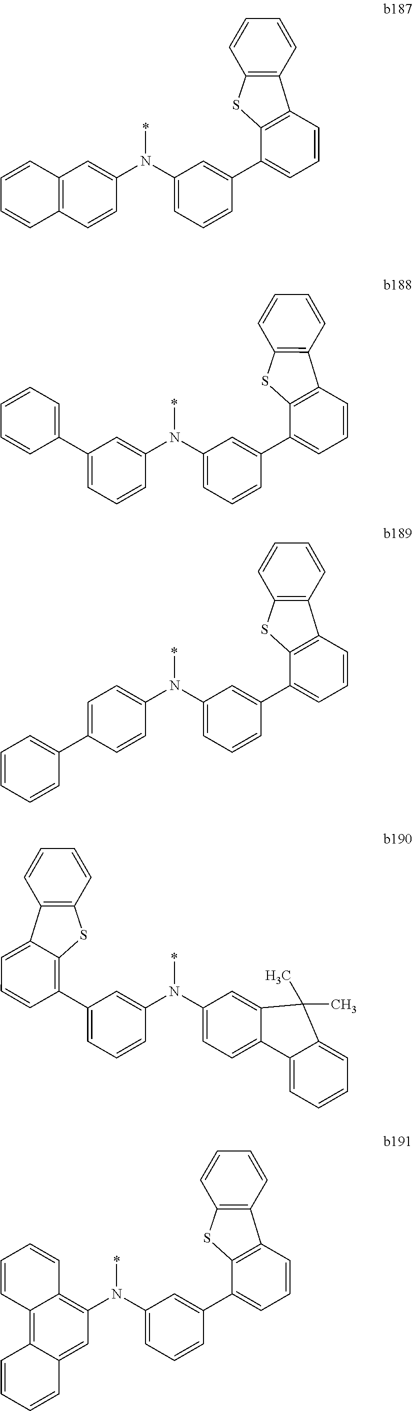 [SMILES: *N(c1cccc(-c2cccc3c2sc2ccccc23)c1)c1ccc2ccccc2c1 ; *N(c1cccc(-c2ccccc2)c1)c1cccc(-c2cccc3c2sc2ccccc23)c1 ; *N(c1ccc(-c2ccccc2)cc1)c1cccc(-c2cccc3c2sc2ccccc23)c1 ; *N(c1cccc(-c2cccc3c2sc2ccccc23)c1)c1ccc2c(c1)C(C)(C)c1ccccc1-2 ; *N(c1cccc(-c2cccc3c2sc2ccccc23)c1)c1cc2ccccc2c2ccccc12]

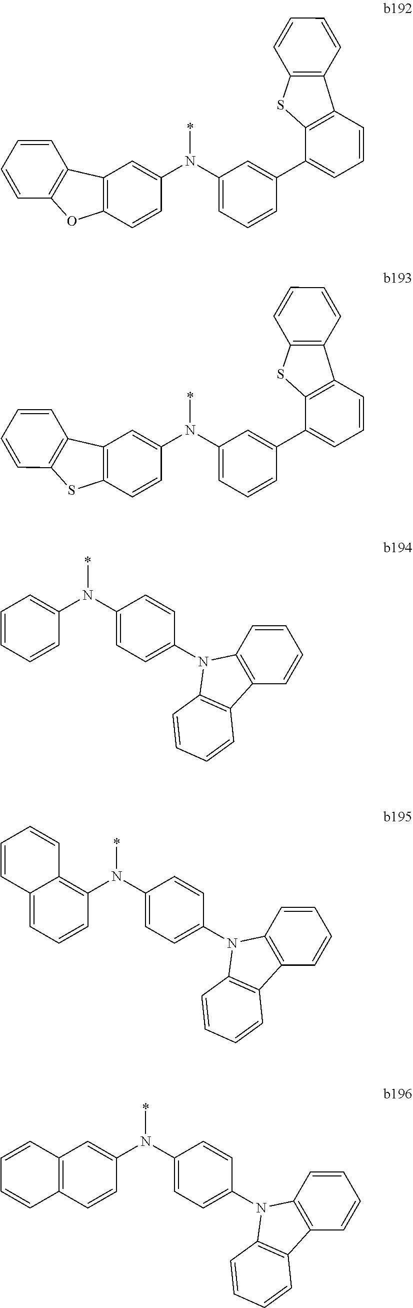 [SMILES: *N(c1cccc(-c2cccc3c2sc2ccccc23)c1)c1ccc2oc3ccccc3c2c1 ; *N(c1cccc(-c2cccc3c2sc2ccccc23)c1)c1ccc2sc3ccccc3c2c1 ; *N(c1ccccc1)c1ccc(-n2c3ccccc3c3ccccc32)cc1 ; *N(c1ccc(-n2c3ccccc3c3ccccc32)cc1)c1cccc2ccccc12 ; *N(c1ccc(-n2c3ccccc3c3ccccc32)cc1)c1ccc2ccccc2c1]

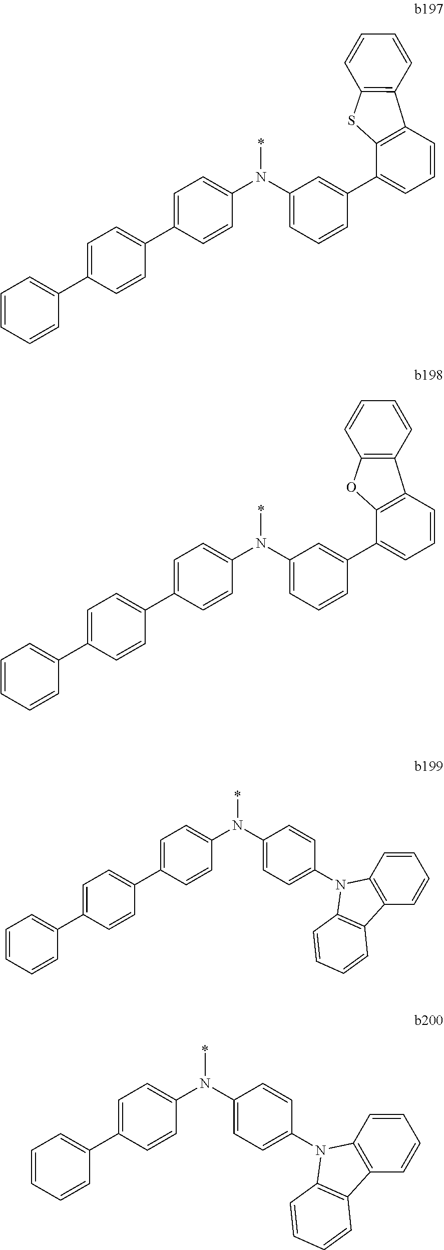 [SMILES: *N(c1ccc(-c2ccc(-c3ccccc3)cc2)cc1)c1cccc(-c2cccc3c2sc2ccccc23)c1 ; *N(c1ccc(-c2ccc(-c3ccccc3)cc2)cc1)c1cccc(-c2cccc3c2oc2ccccc23)c1 ; *N(c1ccc(-c2ccc(-c3ccccc3)cc2)cc1)c1ccc(-n2c3ccccc3c3ccccc32)cc1 ; *N(c1ccc(-c2ccccc2)cc1)c1ccc(-n2c3ccccc3c3ccccc32)cc1]

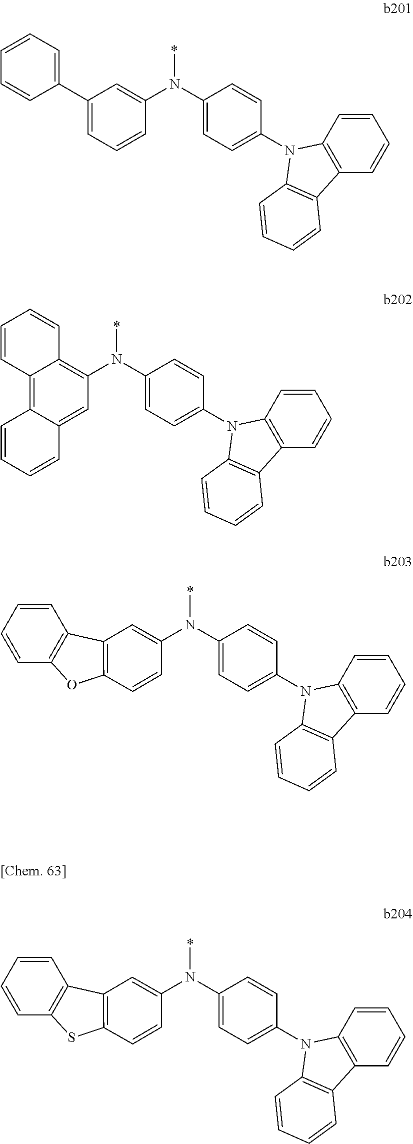 [SMILES: *N(c1ccc(-n2c3ccccc3c3ccccc32)cc1)c1cccc(-c2ccccc2)c1 ; *N(c1ccc(-n2c3ccccc3c3ccccc32)cc1)c1cc2ccccc2c2ccccc12 ; *N(c1ccc(-n2c3ccccc3c3ccccc32)cc1)c1ccc2oc3ccccc3c2c1 ; *N(c1ccc(-n2c3ccccc3c3ccccc32)cc1)c1ccc2sc3ccccc3c2c1]

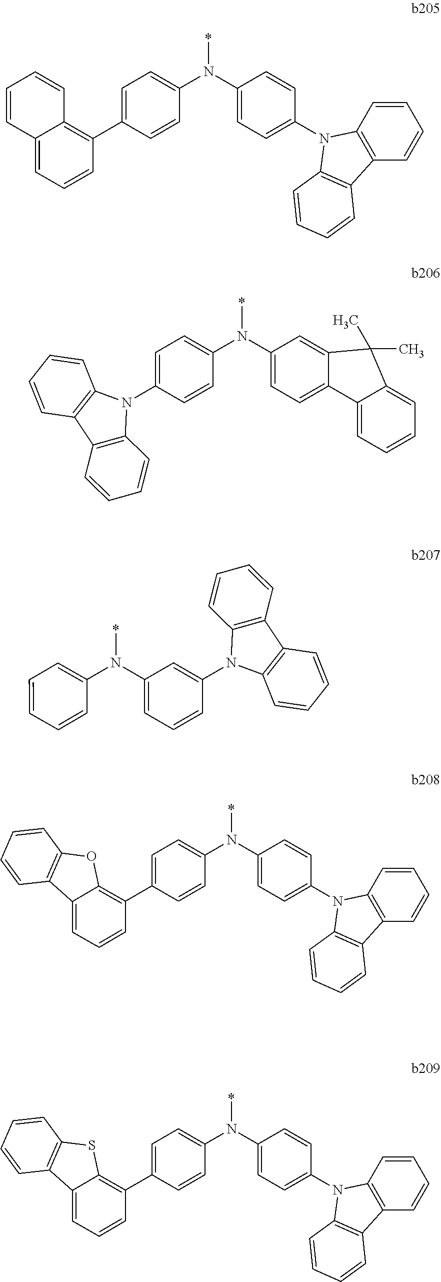 [SMILES: *N(c1ccc(-c2cccc3ccccc23)cc1)c1ccc(-n2c3ccccc3c3ccccc32)cc1 ; *N(c1ccc(-n2c3ccccc3c3ccccc32)cc1)c1ccc2c(c1)C(C)(C)c1ccccc1-2 ; *N(c1ccccc1)c1cccc(-n2c3ccccc3c3ccccc32)c1 ; *N(c1ccc(-c2cccc3c2oc2ccccc23)cc1)c1ccc(-n2c3ccccc3c3ccccc32)cc1 ; *N(c1ccc(-c2cccc3c2sc2ccccc23)cc1)c1ccc(-n2c3ccccc3c3ccccc32)cc1]

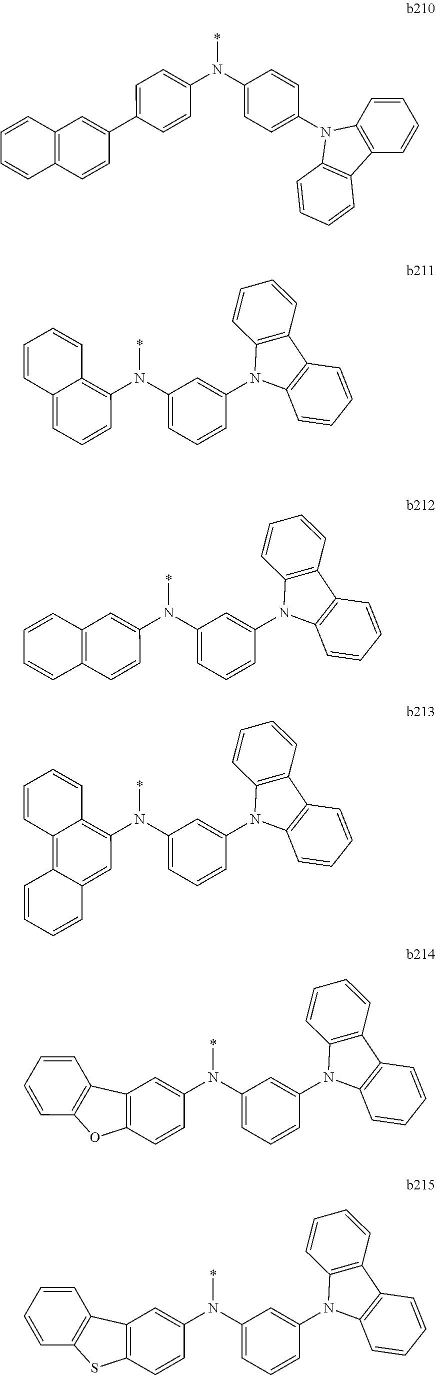 [SMILES: *N(c1ccc(-c2ccc3ccccc3c2)cc1)c1ccc(-n2c3ccccc3c3ccccc32)cc1 ; *N(c1cccc(-n2c3ccccc3c3ccccc32)c1)c1cccc2ccccc12 ; *N(c1cccc(-n2c3ccccc3c3ccccc32)c1)c1ccc2ccccc2c1 ; *N(c1cccc(-n2c3ccccc3c3ccccc32)c1)c1cc2ccccc2c2ccccc12 ; *N(c1cccc(-n2c3ccccc3c3ccccc32)c1)c1ccc2oc3ccccc3c2c1 ; *N(c1cccc(-n2c3ccccc3c3ccccc32)c1)c1ccc2sc3ccccc3c2c1]

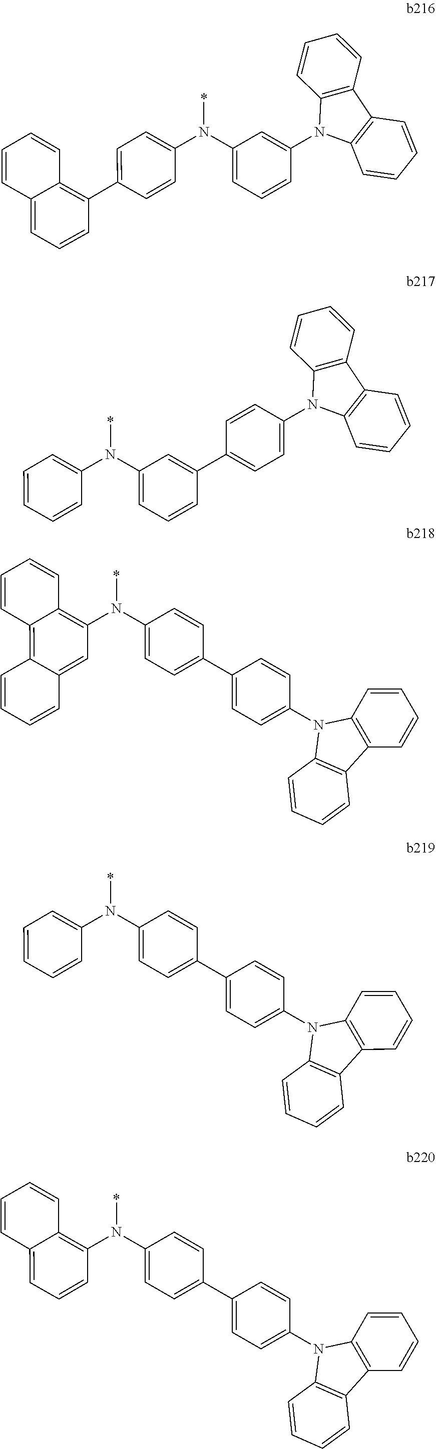

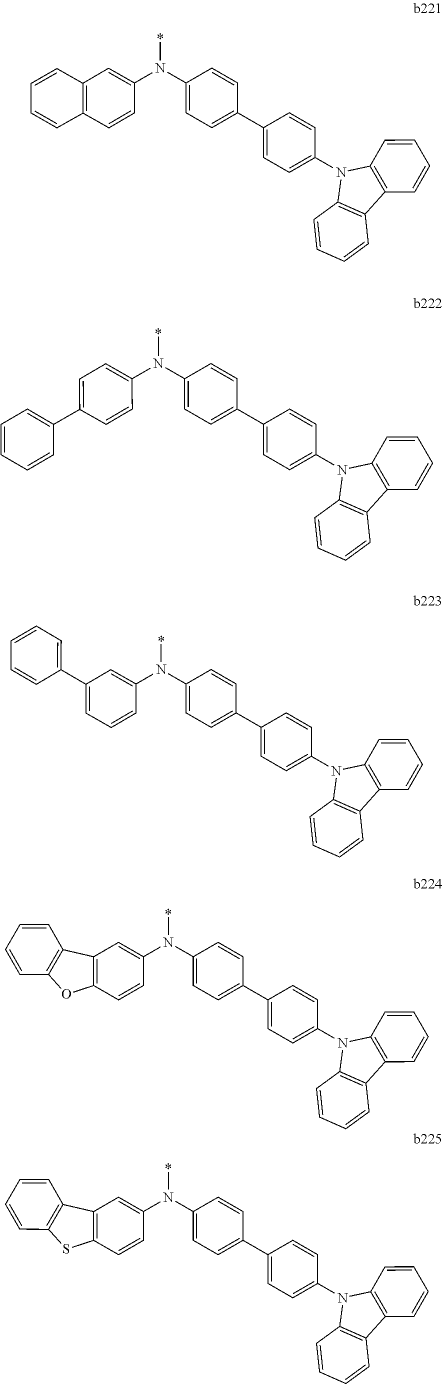 [SMILES: *N(c1ccc(-c2ccc(-n3c4ccccc4c4ccccc43)cc2)cc1)c1ccc2ccccc2c1 ; *N(c1ccc(-c2ccccc2)cc1)c1ccc(-c2ccc(-n3c4ccccc4c4ccccc43)cc2)cc1 ; *N(c1ccc(-c2ccc(-n3c4ccccc4c4ccccc43)cc2)cc1)c1cccc(-c2ccccc2)c1 ; *N(c1ccc(-c2ccc(-n3c4ccccc4c4ccccc43)cc2)cc1)c1ccc2oc3ccccc3c2c1 ; *N(c1ccc(-c2ccc(-n3c4ccccc4c4ccccc43)cc2)cc1)c1ccc2sc3ccccc3c2c1]

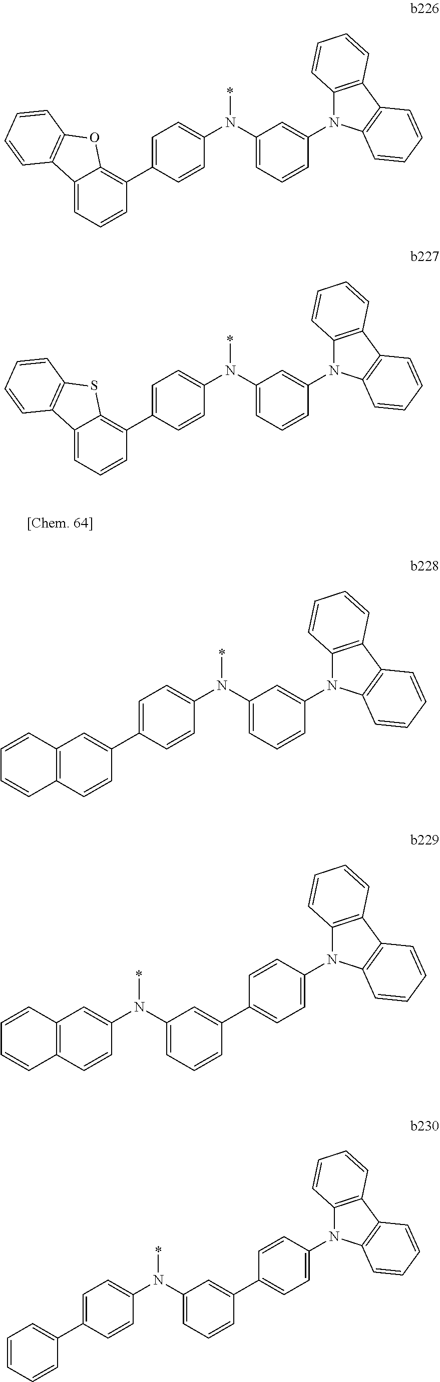 [SMILES: *N(c1ccc(-c2cccc3c2oc2ccccc23)cc1)c1cccc(-n2c3ccccc3c3ccccc32)c1 ; *N(c1ccc(-c2cccc3c2sc2ccccc23)cc1)c1cccc(-n2c3ccccc3c3ccccc32)c1 ; *N(c1ccc(-c2ccc3ccccc3c2)cc1)c1cccc(-n2c3ccccc3c3ccccc32)c1 ; *N(c1cccc(-c2ccc(-n3c4ccccc4c4ccccc43)cc2)c1)c1ccc2ccccc2c1 ; *N(c1ccc(-c2ccccc2)cc1)c1cccc(-c2ccc(-n3c4ccccc4c4ccccc43)cc2)c1]

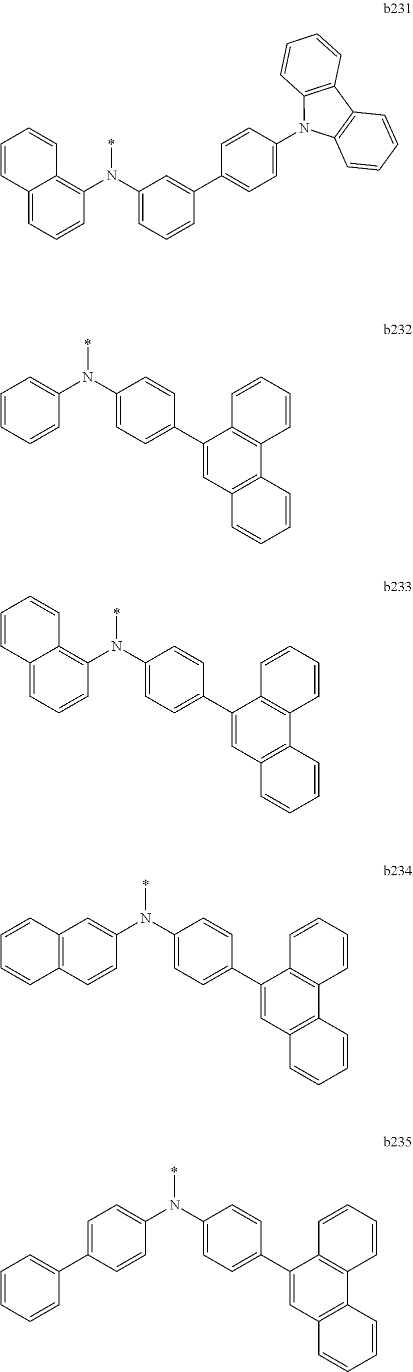 [SMILES: *N(c1cccc(-c2ccc(-n3c4ccccc4c4ccccc43)cc2)c1)c1cccc2ccccc12 ; *N(c1ccccc1)c1ccc(-c2cc3ccccc3c3ccccc23)cc1 ; *N(c1ccc(-c2cc3ccccc3c3ccccc23)cc1)c1cccc2ccccc12 ; *N(c1ccc(-c2cc3ccccc3c3ccccc23)cc1)c1ccc2ccccc2c1 ; *N(c1ccc(-c2ccccc2)cc1)c1ccc(-c2cc3ccccc3c3ccccc23)cc1]

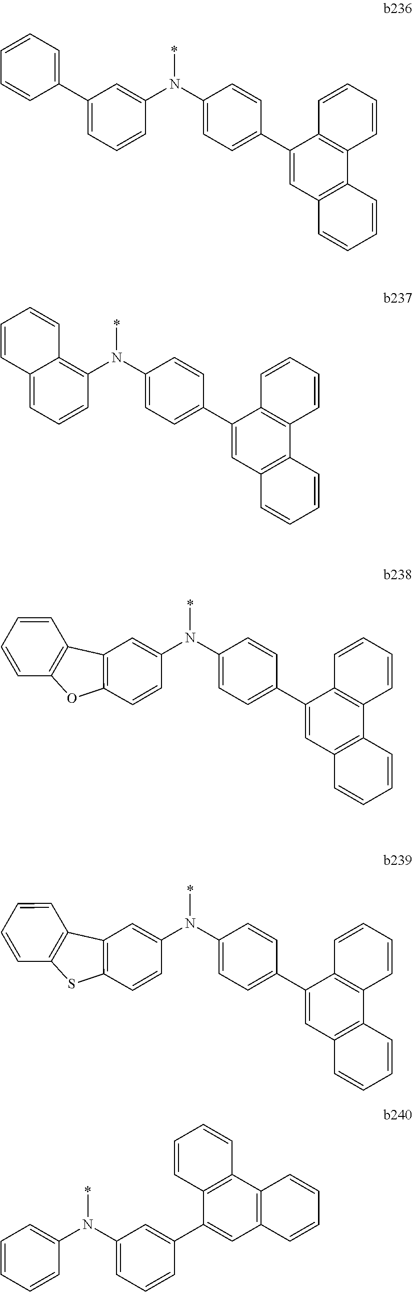 [SMILES: *N(c1ccc(-c2cc3ccccc3c3ccccc23)cc1)c1cccc(-c2ccccc2)c1 ; *N(c1ccc(-c2cc3ccccc3c3ccccc23)cc1)c1cccc2ccccc12 ; *N(c1ccc(-c2cc3ccccc3c3ccccc23)cc1)c1ccc2oc3ccccc3c2c1 ; *N(c1ccc(-c2cc3ccccc3c3ccccc23)cc1)c1ccc2sc3ccccc3c2c1 ; *N(c1ccccc1)c1cccc(-c2cc3ccccc3c3ccccc23)c1]

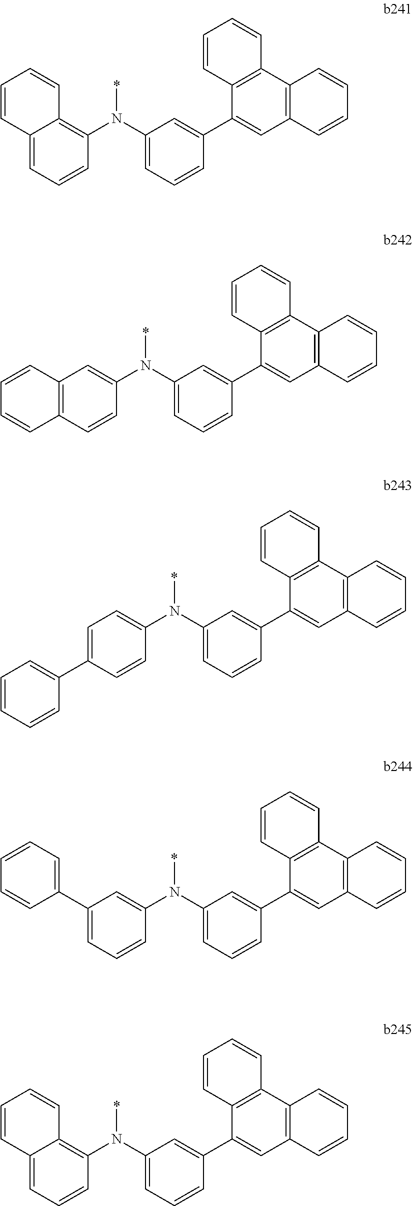 [SMILES: *N(c1cccc(-c2cc3ccccc3c3ccccc23)c1)c1cccc2ccccc12 ; *N(c1cccc(-c2cc3ccccc3c3ccccc23)c1)c1ccc2ccccc2c1 ; *N(c1ccc(-c2ccccc2)cc1)c1cccc(-c2cc3ccccc3c3ccccc23)c1 ; *N(c1cccc(-c2ccccc2)c1)c1cccc(-c2cc3ccccc3c3ccccc23)c1 ; *N(c1cccc(-c2cc3ccccc3c3ccccc23)c1)c1cccc2ccccc12]

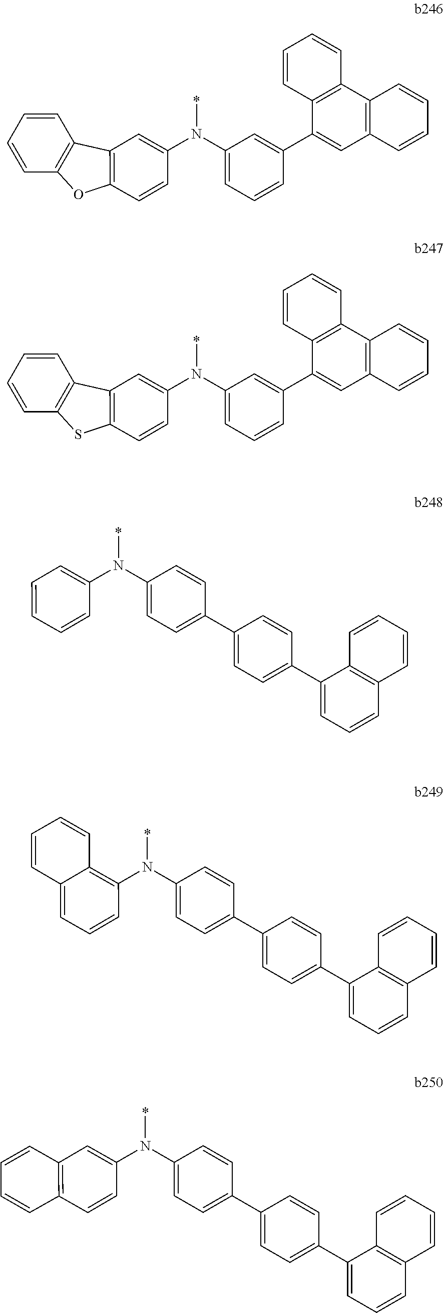 [SMILES: *N(c1cccc(-c2cc3ccccc3c3ccccc23)c1)c1ccc2oc3ccccc3c2c1 ; *N(c1cccc(-c2cc3ccccc3c3ccccc23)c1)c1ccc2sc3ccccc3c2c1 ; *N(c1ccccc1)c1ccc(-c2ccc(-c3cccc4ccccc34)cc2)cc1 ; *N(c1ccc(-c2ccc(-c3cccc4ccccc34)cc2)cc1)c1cccc2ccccc12 ; *N(c1ccc(-c2ccc(-c3cccc4ccccc34)cc2)cc1)c1ccc2ccccc2c1]

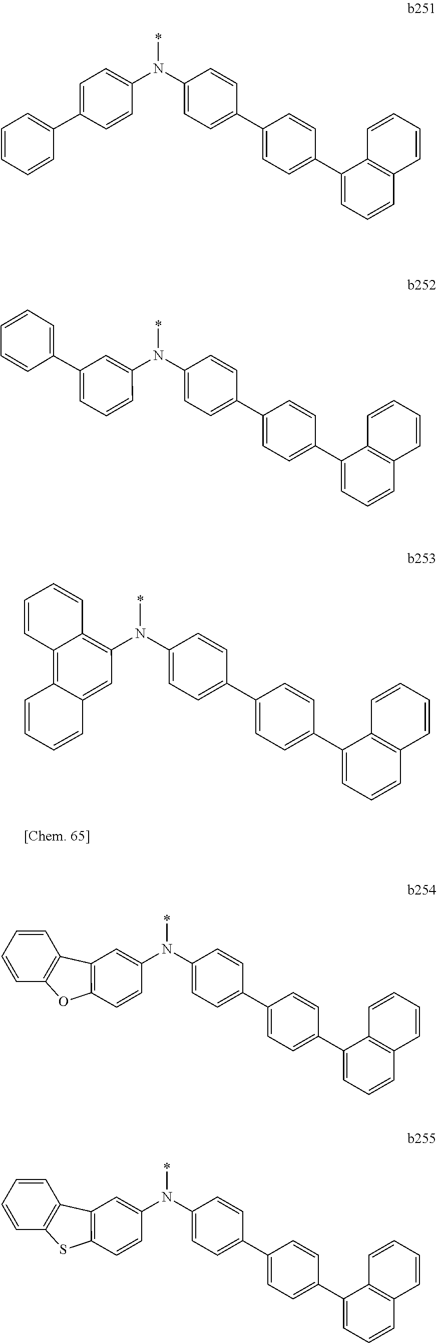 [SMILES: *N(c1ccc(-c2ccccc2)cc1)c1ccc(-c2ccc(-c3cccc4ccccc34)cc2)cc1 ; *N(c1ccc(-c2ccc(-c3cccc4ccccc34)cc2)cc1)c1cccc(-c2ccccc2)c1 ; *N(c1ccc(-c2ccc(-c3cccc4ccccc34)cc2)cc1)c1cc2ccccc2c2ccccc12 ; *N(c1ccc(-c2ccc(-c3cccc4ccccc34)cc2)cc1)c1ccc2oc3ccccc3c2c1 ; *N(c1ccc(-c2ccc(-c3cccc4ccccc34)cc2)cc1)c1ccc2sc3ccccc3c2c1]

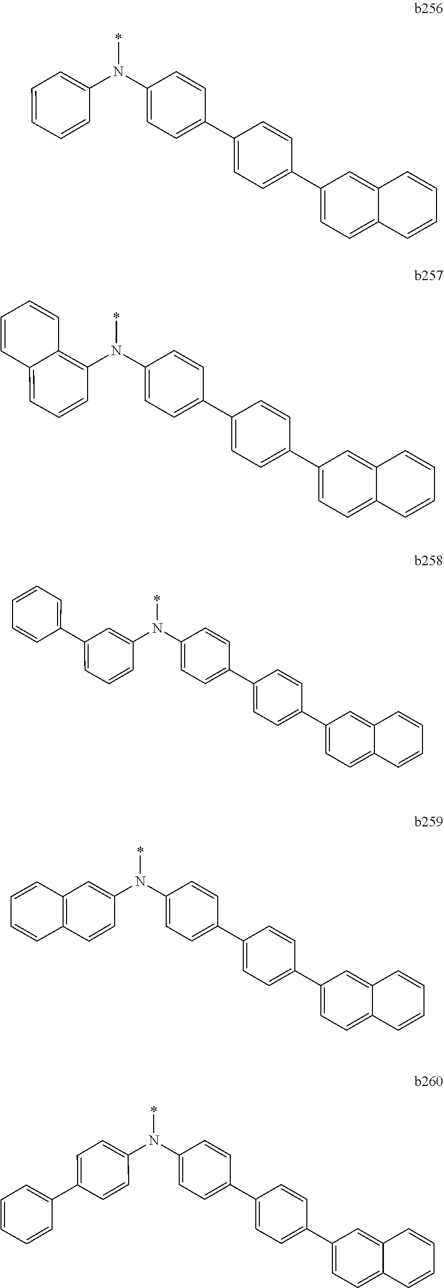 [SMILES: *N(c1ccccc1)c1ccc(-c2ccc(-c3ccc4ccccc4c3)cc2)cc1 ; *N(c1ccc(-c2ccc(-c3ccc4ccccc4c3)cc2)cc1)c1cccc2ccccc12 ; *N(c1ccc(-c2ccc(-c3ccc4ccccc4c3)cc2)cc1)c1cccc(-c2ccccc2)c1 ; *N(c1ccc(-c2ccc(-c3ccc4ccccc4c3)cc2)cc1)c1ccc2ccccc2c1 ; *N(c1ccc(-c2ccccc2)cc1)c1ccc(-c2ccc(-c3ccc4ccccc4c3)cc2)cc1]

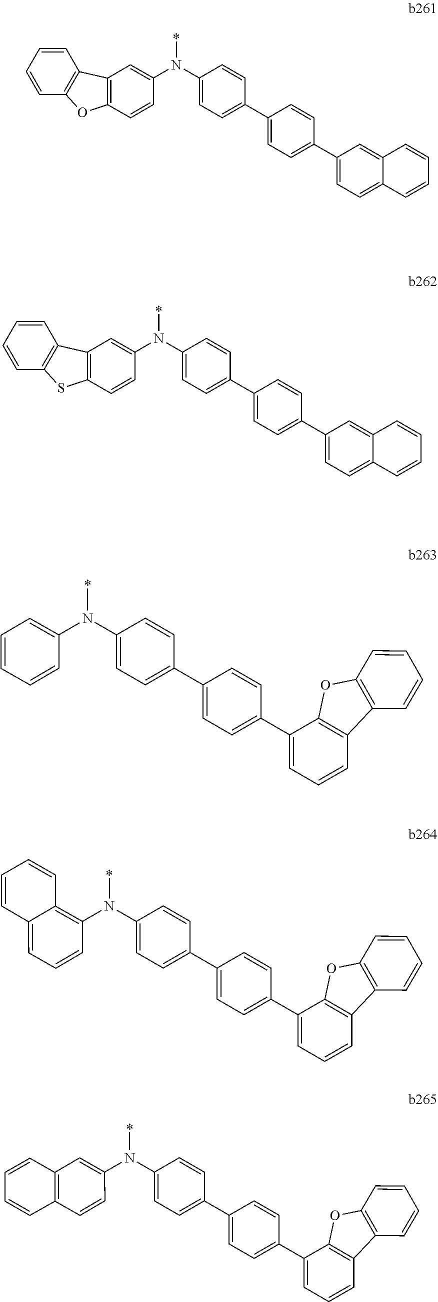 [SMILES: *N(c1ccc(-c2ccc(-c3ccc4ccccc4c3)cc2)cc1)c1ccc2oc3ccccc3c2c1 ; *N(c1ccc(-c2ccc(-c3ccc4ccccc4c3)cc2)cc1)c1ccc2sc3ccccc3c2c1 ; *N(c1ccccc1)c1ccc(-c2ccc(-c3cccc4c3oc3ccccc34)cc2)cc1 ; *N(c1ccc(-c2ccc(-c3cccc4c3oc3ccccc34)cc2)cc1)c1cccc2ccccc12 ; *N(c1ccc(-c2ccc(-c3cccc4c3oc3ccccc34)cc2)cc1)c1ccc2ccccc2c1]

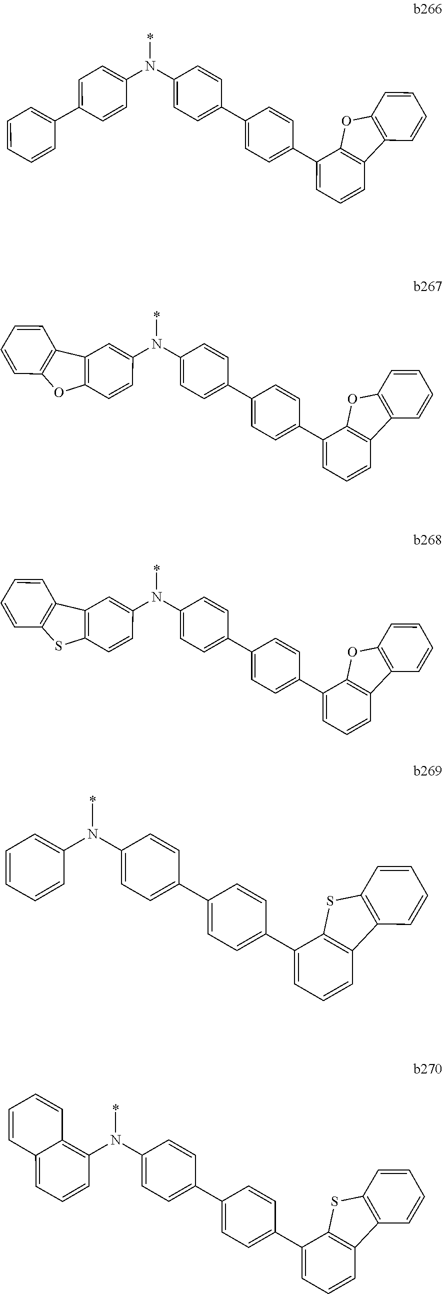 [SMILES: *N(c1ccc(-c2ccccc2)cc1)c1ccc(-c2ccc(-c3cccc4c3oc3ccccc34)cc2)cc1 ; *N(c1ccc(-c2ccc(-c3cccc4c3oc3ccccc34)cc2)cc1)c1ccc2oc3ccccc3c2c1 ; *N(c1ccc(-c2ccc(-c3cccc4c3oc3ccccc34)cc2)cc1)c1ccc2sc3ccccc3c2c1 ; *N(c1ccccc1)c1ccc(-c2ccc(-c3cccc4c3sc3ccccc34)cc2)cc1 ; *N(c1ccc(-c2ccc(-c3cccc4c3sc3ccccc34)cc2)cc1)c1cccc2ccccc12]

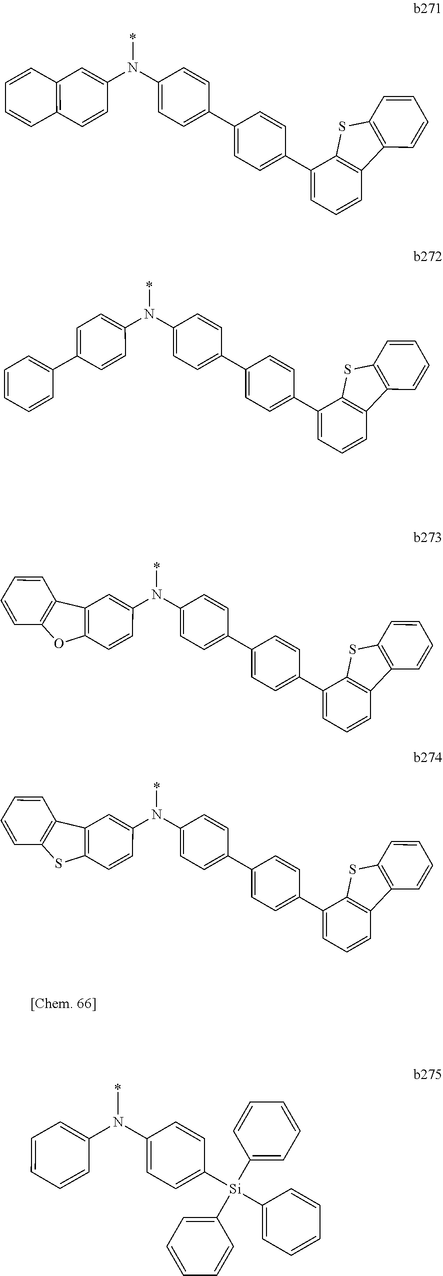 [SMILES: *N(c1ccc(-c2ccc(-c3cccc4c3sc3ccccc34)cc2)cc1)c1ccc2ccccc2c1 ; *N(c1ccc(-c2ccccc2)cc1)c1ccc(-c2ccc(-c3cccc4c3sc3ccccc34)cc2)cc1 ; *N(c1ccc(-c2ccc(-c3cccc4c3sc3ccccc34)cc2)cc1)c1ccc2oc3ccccc3c2c1 ; *N(c1ccc(-c2ccc(-c3cccc4c3sc3ccccc34)cc2)cc1)c1ccc2sc3ccccc3c2c1 ; *N(c1ccccc1)c1ccc([Si](c2ccccc2)(c2ccccc2)c2ccccc2)cc1]

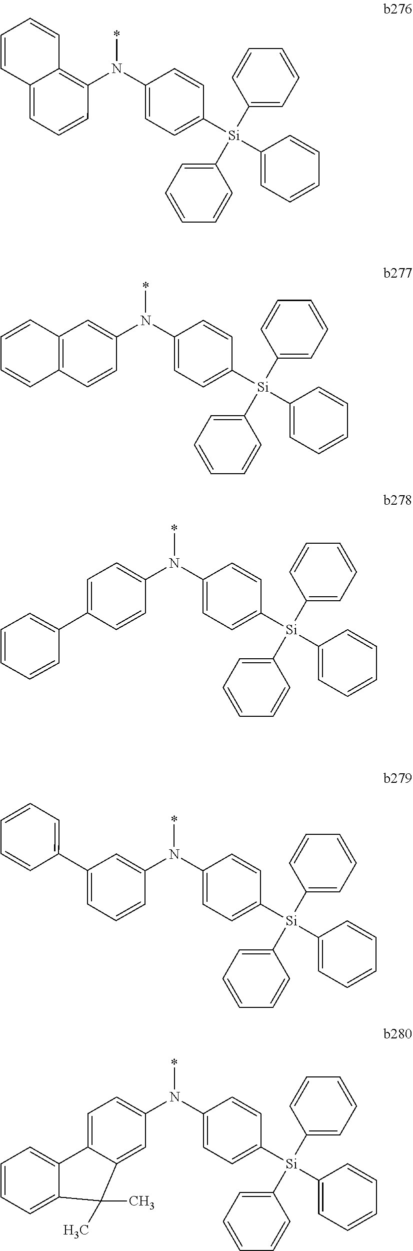 [SMILES: *N(c1ccc([Si](c2ccccc2)(c2ccccc2)c2ccccc2)cc1)c1cccc2ccccc12 ; *N(c1ccc([Si](c2ccccc2)(c2ccccc2)c2ccccc2)cc1)c1ccc2ccccc2c1 ; *N(c1ccc(-c2ccccc2)cc1)c1ccc([Si](c2ccccc2)(c2ccccc2)c2ccccc2)cc1 ; *N(c1ccc([Si](c2ccccc2)(c2ccccc2)c2ccccc2)cc1)c1cccc(-c2ccccc2)c1 ; *N(c1ccc([Si](c2ccccc2)(c2ccccc2)c2ccccc2)cc1)c1ccc2c(c1)C(C)(C)c1ccccc1-2]

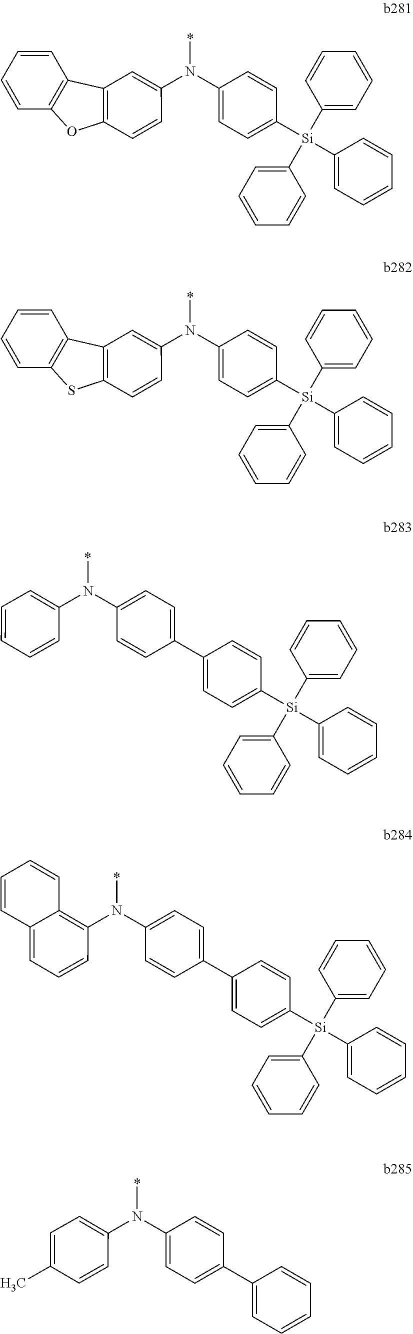 [SMILES: *N(c1ccc([Si](c2ccccc2)(c2ccccc2)c2ccccc2)cc1)c1ccc2oc3ccccc3c2c1 ; *N(c1ccc([Si](c2ccccc2)(c2ccccc2)c2ccccc2)cc1)c1ccc2sc3ccccc3c2c1 ; *N(c1ccccc1)c1ccc(-c2ccc([Si](c3ccccc3)(c3ccccc3)c3ccccc3)cc2)cc1 ; *N(c1ccc(-c2ccc([Si](c3ccccc3)(c3ccccc3)c3ccccc3)cc2)cc1)c1cccc2ccccc12 ; *N(c1ccc(C)cc1)c1ccc(-c2ccccc2)cc1]

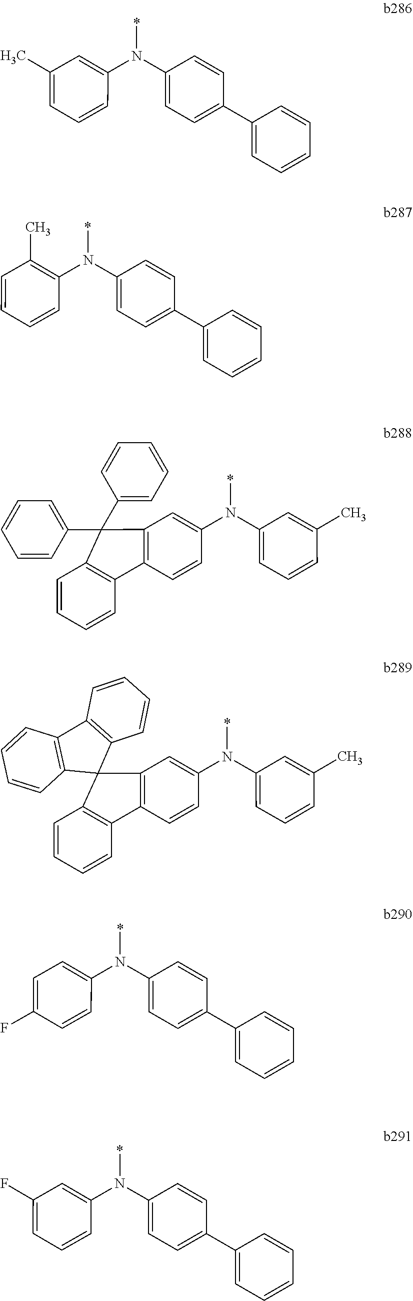 [SMILES: *N(c1ccc(-c2ccccc2)cc1)c1cccc(C)c1 ; *N(c1ccc(-c2ccccc2)cc1)c1ccccc1C ; *N(c1cccc(C)c1)c1ccc2c(c1)C(c1ccccc1)(c1ccccc1)c1ccccc1-2 ; *N(c1cccc(C)c1)c1ccc2c(c1)C1(c3ccccc3-c3ccccc31)c1ccccc1-2 ; *N(c1ccc(F)cc1)c1ccc(-c2ccccc2)cc1 ; *N(c1ccc(-c2ccccc2)cc1)c1cccc(F)c1]

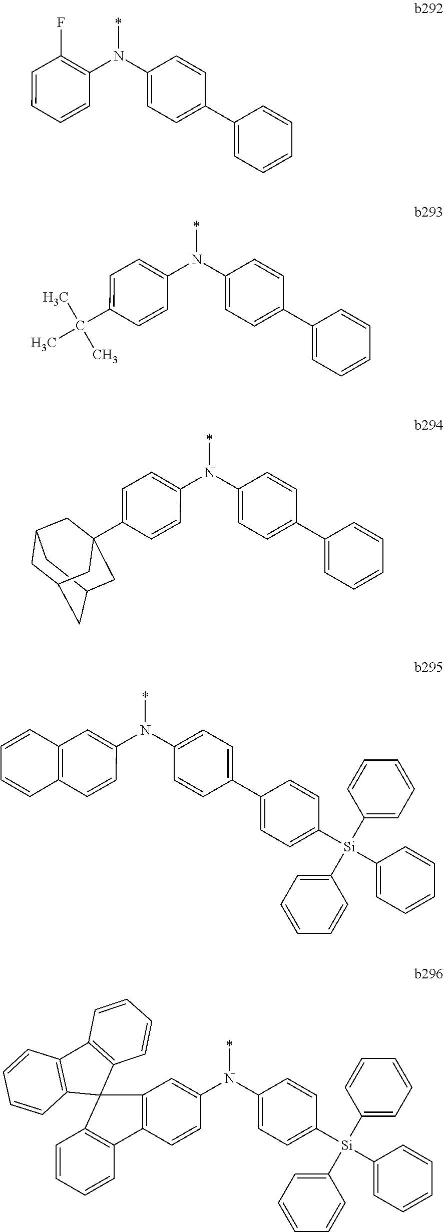 [SMILES: *N(c1ccc(-c2ccccc2)cc1)c1ccccc1F ; *N(c1ccc(-c2ccccc2)cc1)c1ccc(C(C)(C)C)cc1 ; *N(c1ccc(-c2ccccc2)cc1)c1ccc(C23CC4CC(CC(C4)C2)C3)cc1 ; *N(c1ccc(-c2ccc([Si](c3ccccc3)(c3ccccc3)c3ccccc3)cc2)cc1)c1ccc2ccccc2c1 ; *N(c1ccc([Si](c2ccccc2)(c2ccccc2)c2ccccc2)cc1)c1ccc2c(c1)C1(c3ccccc3-c3ccccc31)c1ccccc1-2]

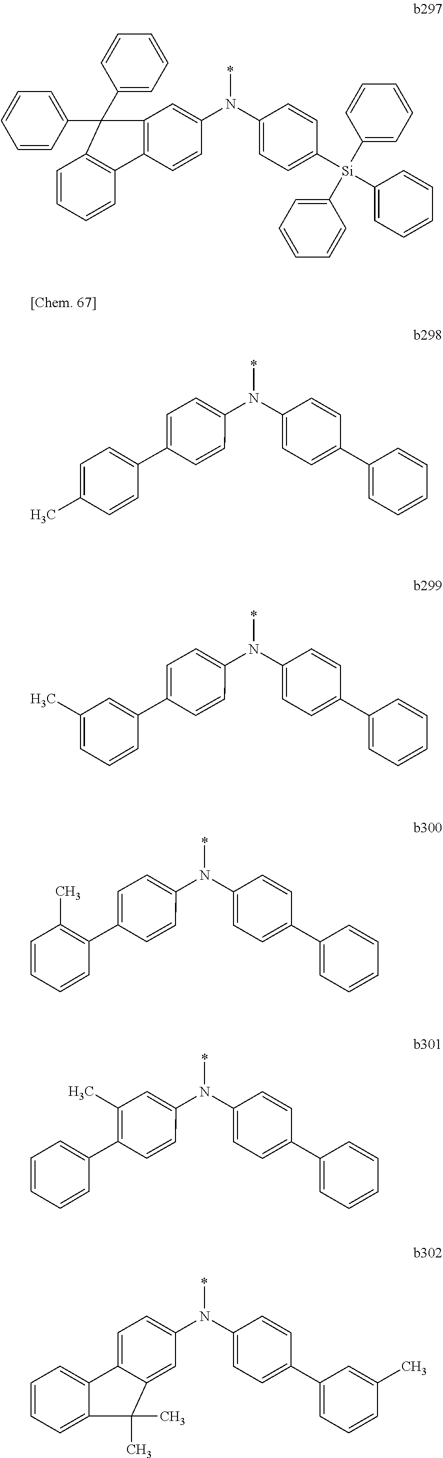 [SMILES: *N(c1ccc([Si](c2ccccc2)(c2ccccc2)c2ccccc2)cc1)c1ccc2c(c1)C(c1ccccc1)(c1ccccc1)c1ccccc1-2 ; *N(c1ccc(-c2ccccc2)cc1)c1ccc(-c2ccc(C)cc2)cc1 ; *N(c1ccc(-c2ccccc2)cc1)c1ccc(-c2cccc(C)c2)cc1 ; *N(c1ccc(-c2ccccc2)cc1)c1ccc(-c2ccccc2C)cc1 ; *N(c1ccc(-c2ccccc2)cc1)c1ccc(-c2ccccc2)c(C)c1 ; *N(c1ccc(-c2cccc(C)c2)cc1)c1ccc2c(c1)C(C)(C)c1ccccc1-2]

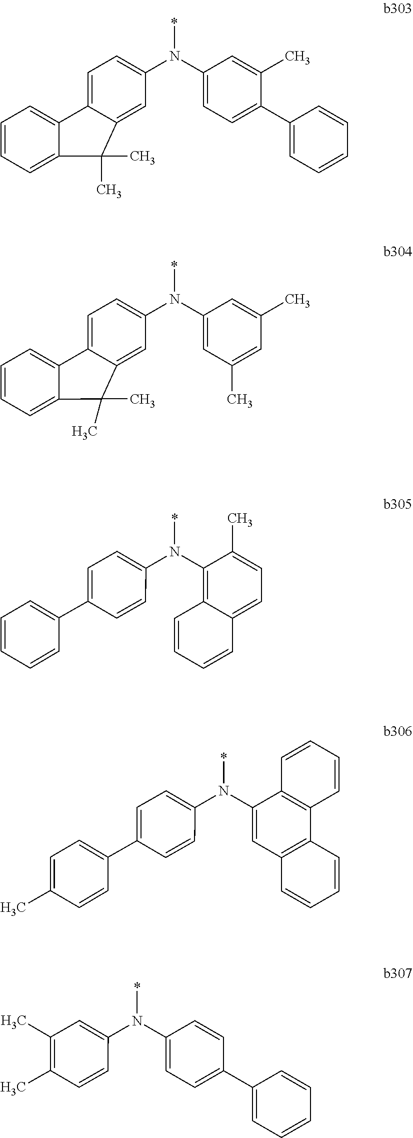

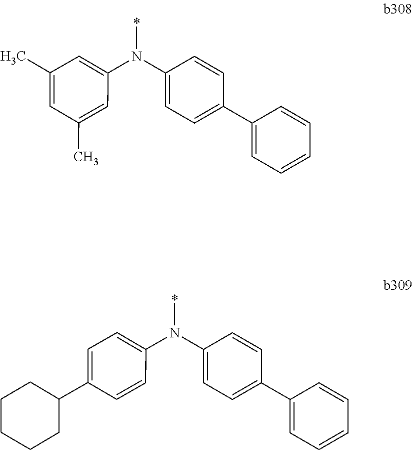 [SMILES: *N(c1ccc(-c2ccccc2)cc1)c1cc(C)cc(C)c1 ; *N(c1ccc(-c2ccccc2)cc1)c1ccc(C2CCCCC2)cc1]

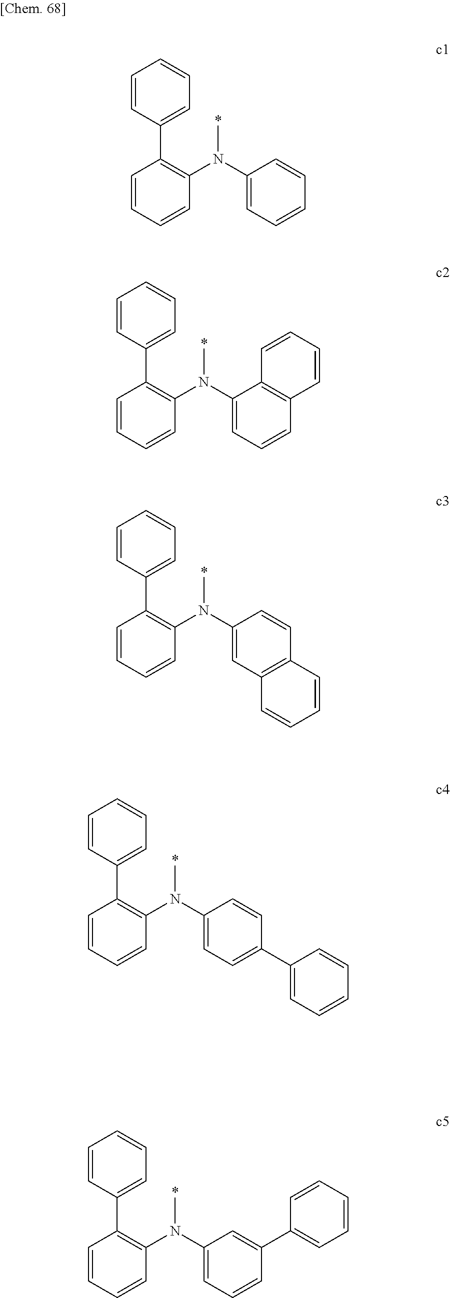 [SMILES: CN(c1ccccc1)c1ccccc1-c1ccccc1 ; CN(c1ccccc1-c1ccccc1)c1cccc2ccccc12 ; CN(c1ccc2ccccc2c1)c1ccccc1-c1ccccc1 ; CN(c1ccc(-c2ccccc2)cc1)c1ccccc1-c1ccccc1 ; CN(c1cccc(-c2ccccc2)c1)c1ccccc1-c1ccccc1]

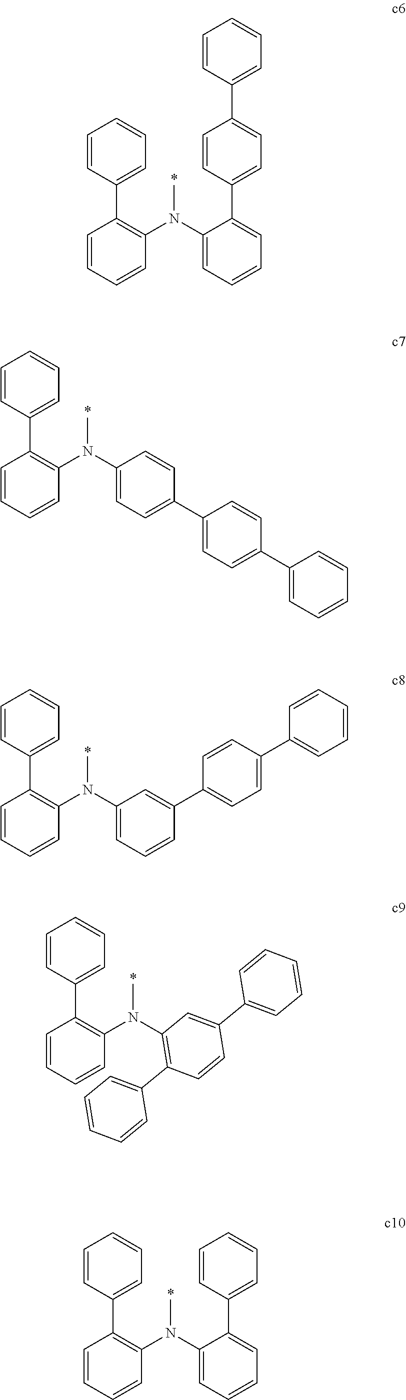 [SMILES: CN(c1ccccc1-c1ccccc1)c1ccccc1-c1ccc(-c2ccccc2)cc1 ; CN(c1ccc(-c2ccc(-c3ccccc3)cc2)cc1)c1ccccc1-c1ccccc1 ; CN(c1cccc(-c2ccc(-c3ccccc3)cc2)c1)c1ccccc1-c1ccccc1 ; CN(c1ccccc1-c1ccccc1)c1cc(-c2ccccc2)ccc1-c1ccccc1 ; CN(c1ccccc1-c1ccccc1)c1ccccc1-c1ccccc1]

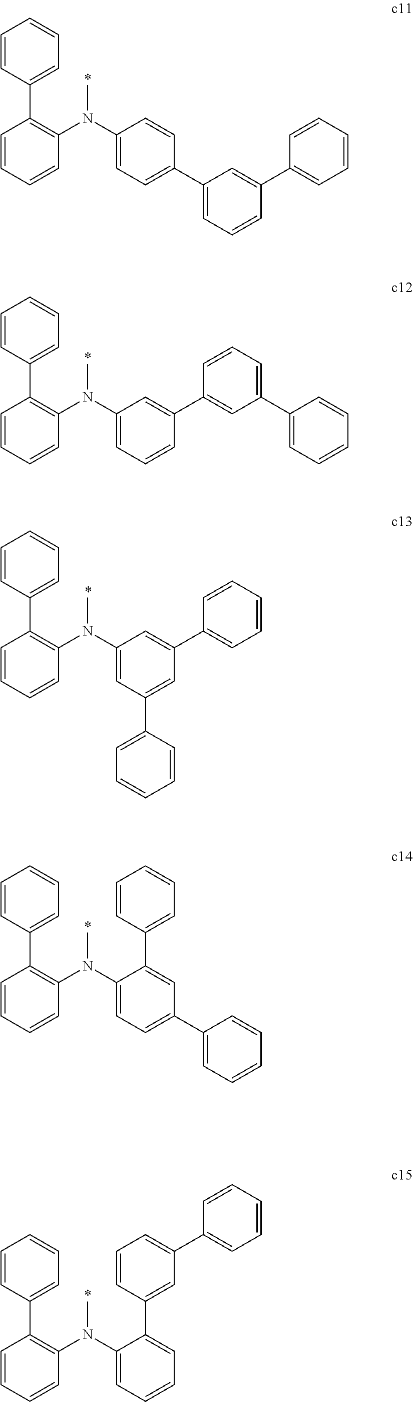 [SMILES: CN(c1ccc(-c2cccc(-c3ccccc3)c2)cc1)c1ccccc1-c1ccccc1 ; CN(c1cccc(-c2cccc(-c3ccccc3)c2)c1)c1ccccc1-c1ccccc1 ; CN(c1cc(-c2ccccc2)cc(-c2ccccc2)c1)c1ccccc1-c1ccccc1 ; CN(c1ccccc1-c1ccccc1)c1ccc(-c2ccccc2)cc1-c1ccccc1 ; CN(c1ccccc1-c1ccccc1)c1ccccc1-c1cccc(-c2ccccc2)c1]

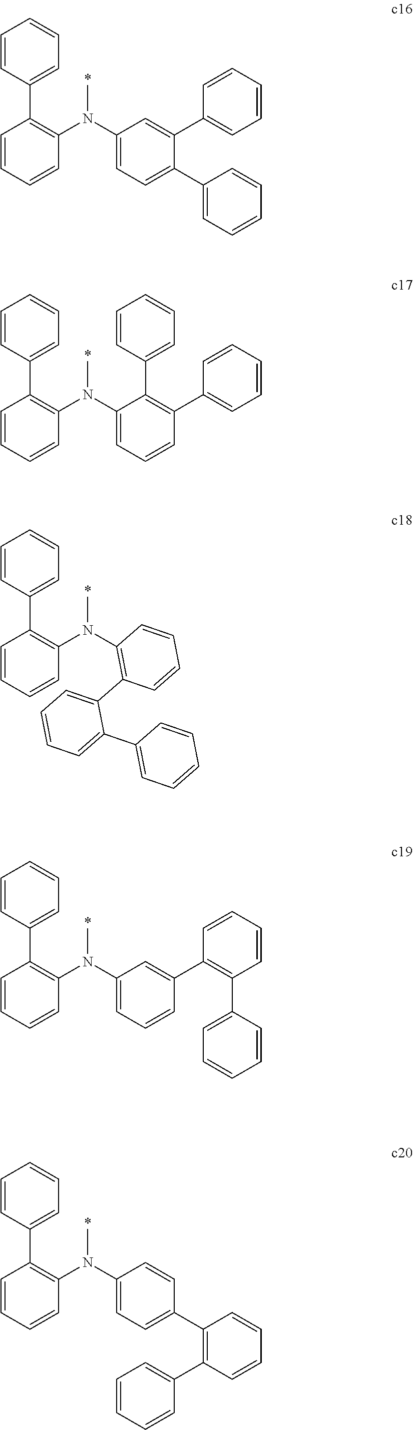 [SMILES: CN(c1ccc(-c2ccccc2)c(-c2ccccc2)c1)c1ccccc1-c1ccccc1 ; CN(c1ccccc1-c1ccccc1)c1cccc(-c2ccccc2)c1-c1ccccc1 ; CN(c1ccccc1-c1ccccc1)c1ccccc1-c1ccccc1-c1ccccc1 ; CN(c1cccc(-c2ccccc2-c2ccccc2)c1)c1ccccc1-c1ccccc1 ; CN(c1ccc(-c2ccccc2-c2ccccc2)cc1)c1ccccc1-c1ccccc1]

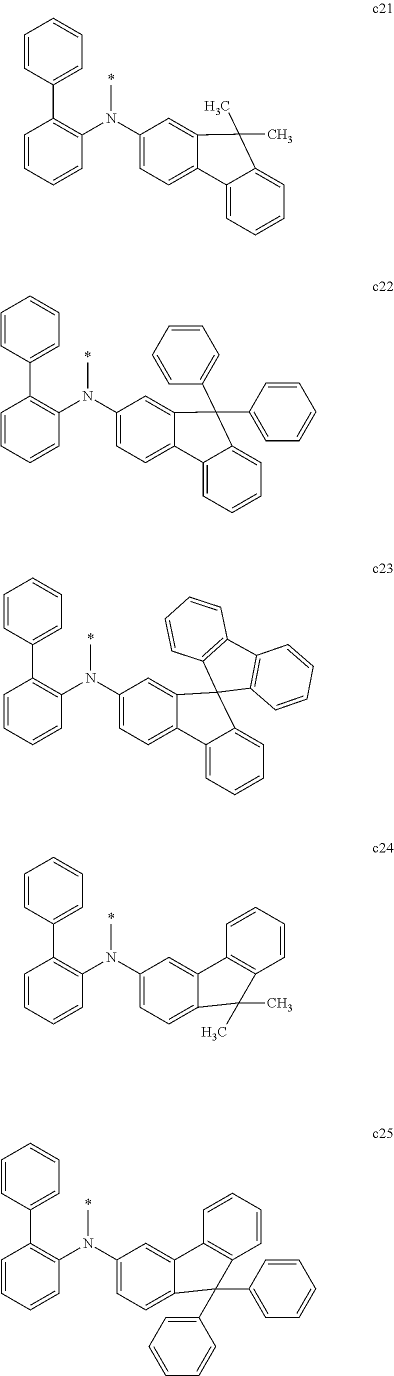 [SMILES: CN(c1ccc2c(c1)C(C)(C)c1ccccc1-2)c1ccccc1-c1ccccc1 ; CN(c1ccc2c(c1)C(c1ccccc1)(c1ccccc1)c1ccccc1-2)c1ccccc1-c1ccccc1 ; CN(c1ccc2c(c1)C1(c3ccccc3-c3ccccc31)c1ccccc1-2)c1ccccc1-c1ccccc1 ; CN(c1ccc2c(c1)-c1ccccc1C2(C)C)c1ccccc1-c1ccccc1 ; CN(c1ccc2c(c1)-c1ccccc1C2(c1ccccc1)c1ccccc1)c1ccccc1-c1ccccc1]

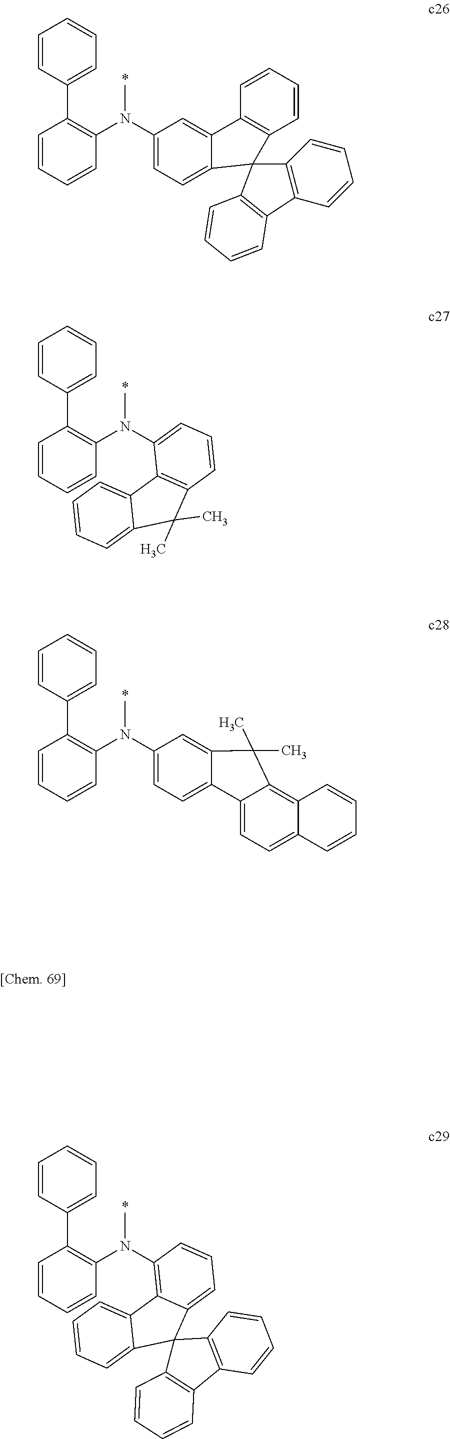 [SMILES: CN(c1ccc2c(c1)-c1ccccc1C21c2ccccc2-c2ccccc21)c1ccccc1-c1ccccc1 ; CN(c1ccccc1-c1ccccc1)c1cccc2c1-c1ccccc1C2(C)C ; CN(c1ccc2c(c1)C(C)(C)c1c-2ccc2ccccc12)c1ccccc1-c1ccccc1 ; CN(c1ccccc1-c1ccccc1)c1cccc2c1-c1ccccc1C21c2ccccc2-c2ccccc21]

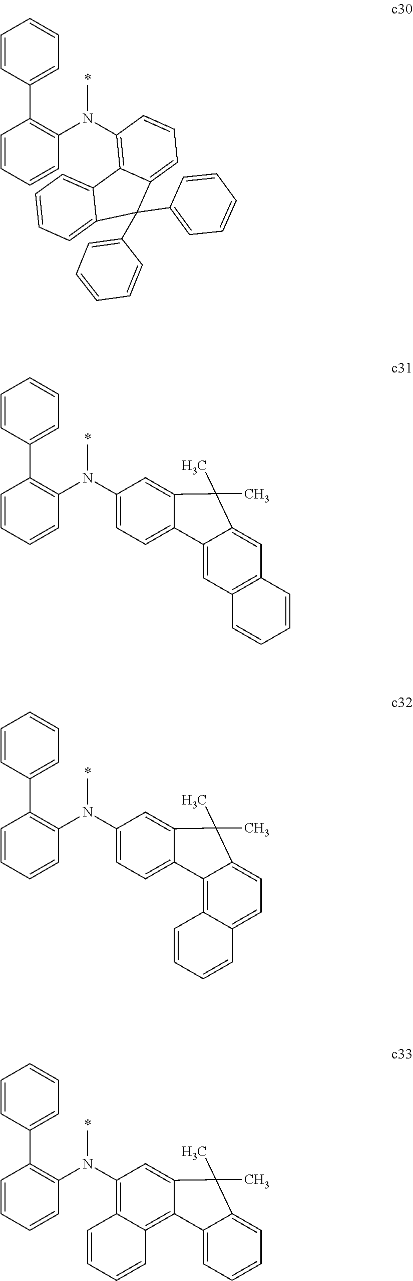 [SMILES: CN(c1ccccc1-c1ccccc1)c1cccc2c1-c1ccccc1C2(c1ccccc1)c1ccccc1 ; CN(c1ccc2c(c1)C(C)(C)c1cc3ccccc3cc1-2)c1ccccc1-c1ccccc1 ; CN(c1ccc2c(c1)C(C)(C)c1ccc3ccccc3c1-2)c1ccccc1-c1ccccc1 ; CN(c1ccccc1-c1ccccc1)c1cc2c(c3ccccc13)-c1ccccc1C2(C)C]

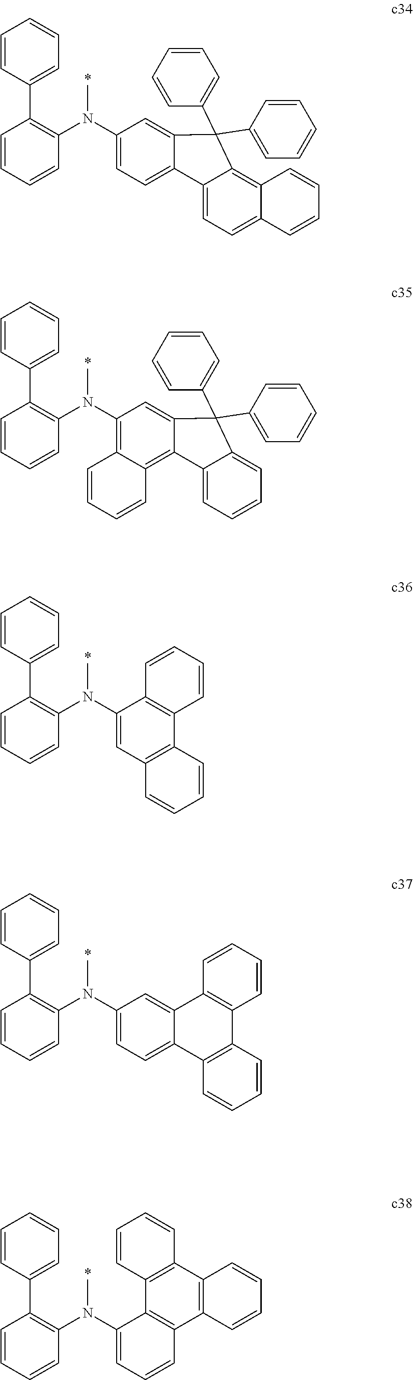 [SMILES: CN(c1ccc2c(c1)C(c1ccccc1)(c1ccccc1)c1c-2ccc2ccccc12)c1ccccc1-c1ccccc1 ; CN(c1ccccc1-c1ccccc1)c1cc2c(c3ccccc13)-c1ccccc1C2(c1ccccc1)c1ccccc1 ; CN(c1ccccc1-c1ccccc1)c1cc2ccccc2c2ccccc12 ; CN(c1ccc2c3ccccc3c3ccccc3c2c1)c1ccccc1-c1ccccc1 ; CN(c1ccccc1-c1ccccc1)c1cccc2c3ccccc3c3ccccc3c12]

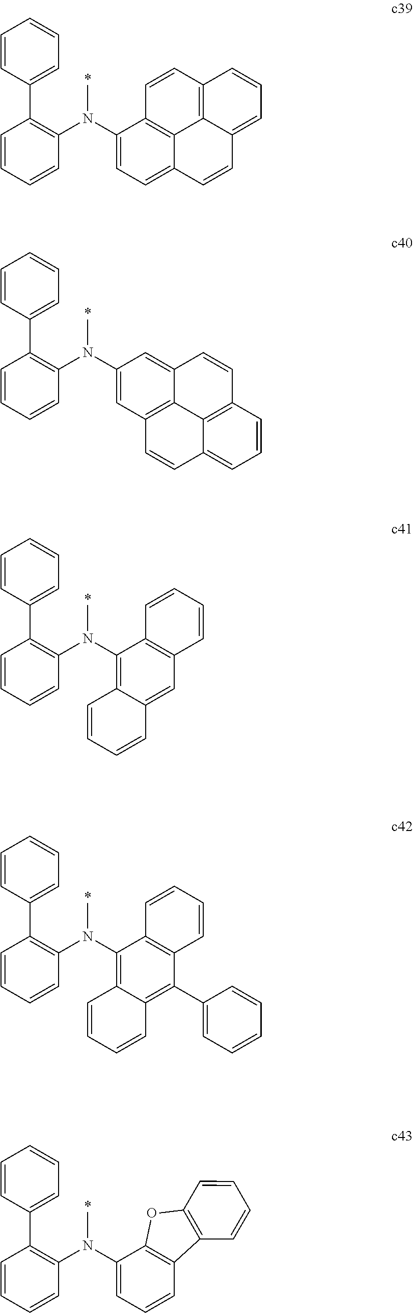 [SMILES: CN(c1ccccc1-c1ccccc1)c1ccc2ccc3cccc4ccc1c2c34 ; CN(c1cc2ccc3cccc4ccc(c1)c2c34)c1ccccc1-c1ccccc1 ; CN(c1ccccc1-c1ccccc1)c1c2ccccc2cc2ccccc12 ; CN(c1ccccc1-c1ccccc1)c1c2ccccc2c(-c2ccccc2)c2ccccc12 ; CN(c1ccccc1-c1ccccc1)c1cccc2c1oc1ccccc12]

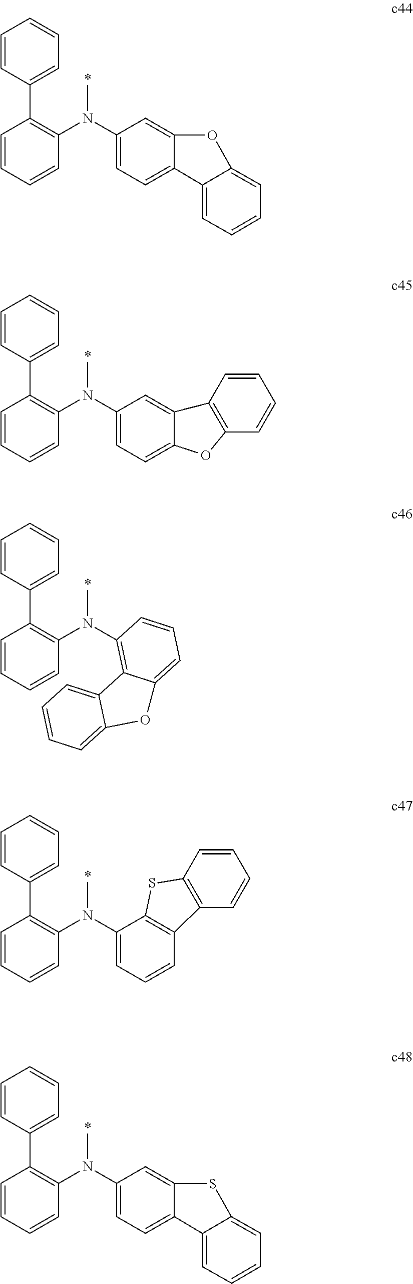 [SMILES: CN(c1ccc2c(c1)oc1ccccc12)c1ccccc1-c1ccccc1 ; CN(c1ccc2oc3ccccc3c2c1)c1ccccc1-c1ccccc1 ; CN(c1ccccc1-c1ccccc1)c1cccc2oc3ccccc3c12 ; CN(c1ccccc1-c1ccccc1)c1cccc2c1sc1ccccc12 ; CN(c1ccc2c(c1)sc1ccccc12)c1ccccc1-c1ccccc1]

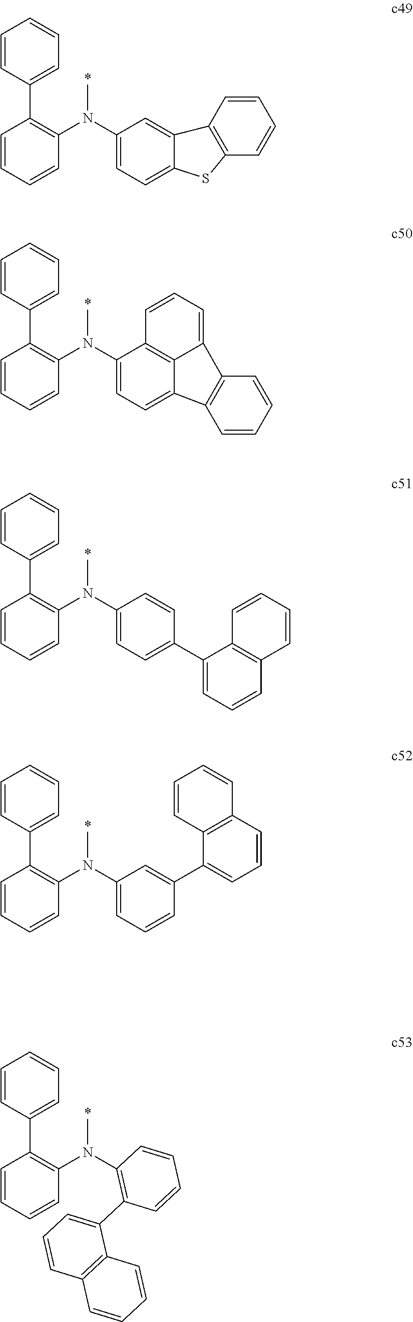 [SMILES: CN(c1ccc2sc3ccccc3c2c1)c1ccccc1-c1ccccc1 ; CN(c1ccccc1-c1ccccc1)c1ccc2c3c(cccc13)-c1ccccc1-2 ; CN(c1ccc(-c2cccc3ccccc23)cc1)c1ccccc1-c1ccccc1 ; CN(c1cccc(-c2cccc3ccccc23)c1)c1ccccc1-c1ccccc1 ; CN(c1ccccc1-c1ccccc1)c1ccccc1-c1cccc2ccccc12]

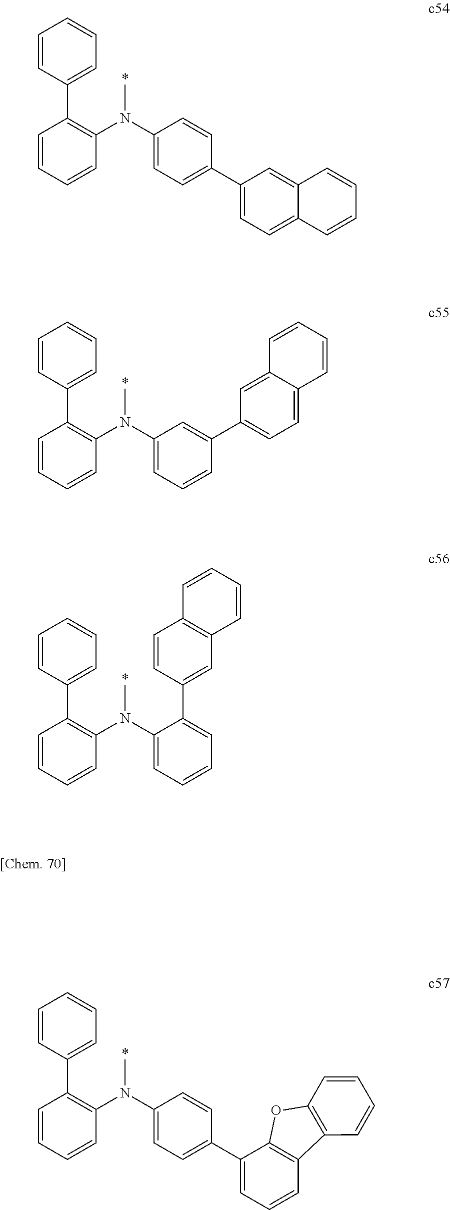 [SMILES: CN(c1ccc(-c2ccc3ccccc3c2)cc1)c1ccccc1-c1ccccc1 ; CN(c1cccc(-c2ccc3ccccc3c2)c1)c1ccccc1-c1ccccc1 ; CN(c1ccccc1-c1ccccc1)c1ccccc1-c1ccc2ccccc2c1 ; CN(c1ccc(-c2cccc3c2oc2ccccc23)cc1)c1ccccc1-c1ccccc1]

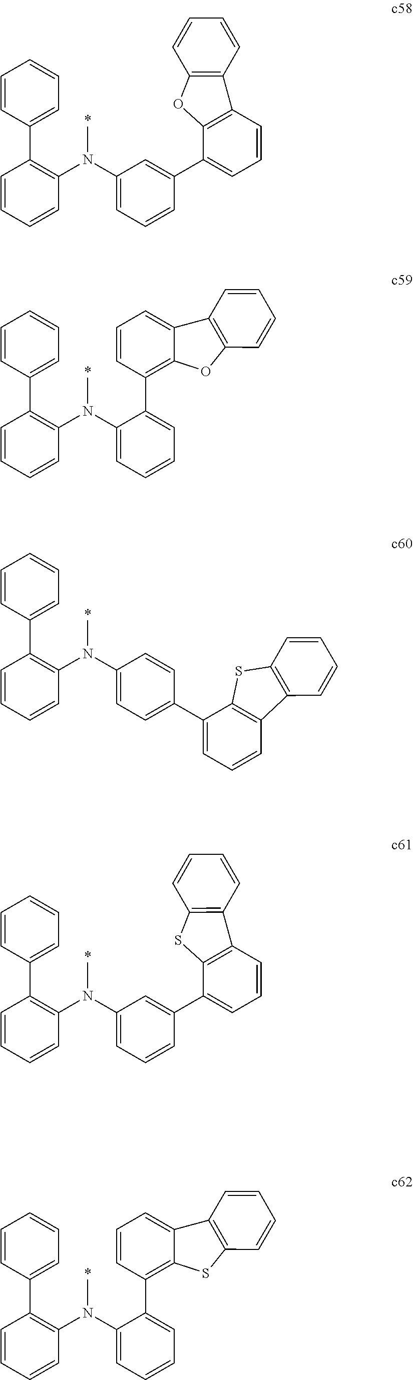 [SMILES: CN(c1cccc(-c2cccc3c2oc2ccccc23)c1)c1ccccc1-c1ccccc1 ; CN(c1ccccc1-c1ccccc1)c1ccccc1-c1cccc2c1oc1ccccc12 ; CN(c1ccc(-c2cccc3c2sc2ccccc23)cc1)c1ccccc1-c1ccccc1 ; CN(c1cccc(-c2cccc3c2sc2ccccc23)c1)c1ccccc1-c1ccccc1 ; CN(c1ccccc1-c1ccccc1)c1ccccc1-c1cccc2c1sc1ccccc12]

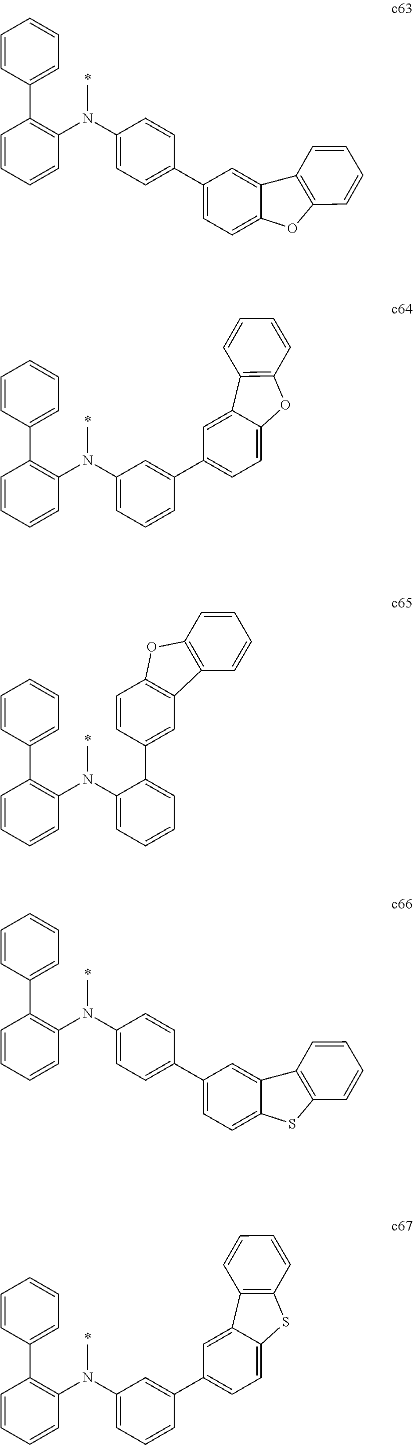 [SMILES: CN(c1ccc(-c2ccc3oc4ccccc4c3c2)cc1)c1ccccc1-c1ccccc1 ; CN(c1cccc(-c2ccc3oc4ccccc4c3c2)c1)c1ccccc1-c1ccccc1 ; CN(c1ccccc1-c1ccccc1)c1ccccc1-c1ccc2oc3ccccc3c2c1 ; CN(c1ccc(-c2ccc3sc4ccccc4c3c2)cc1)c1ccccc1-c1ccccc1 ; CN(c1cccc(-c2ccc3sc4ccccc4c3c2)c1)c1ccccc1-c1ccccc1]

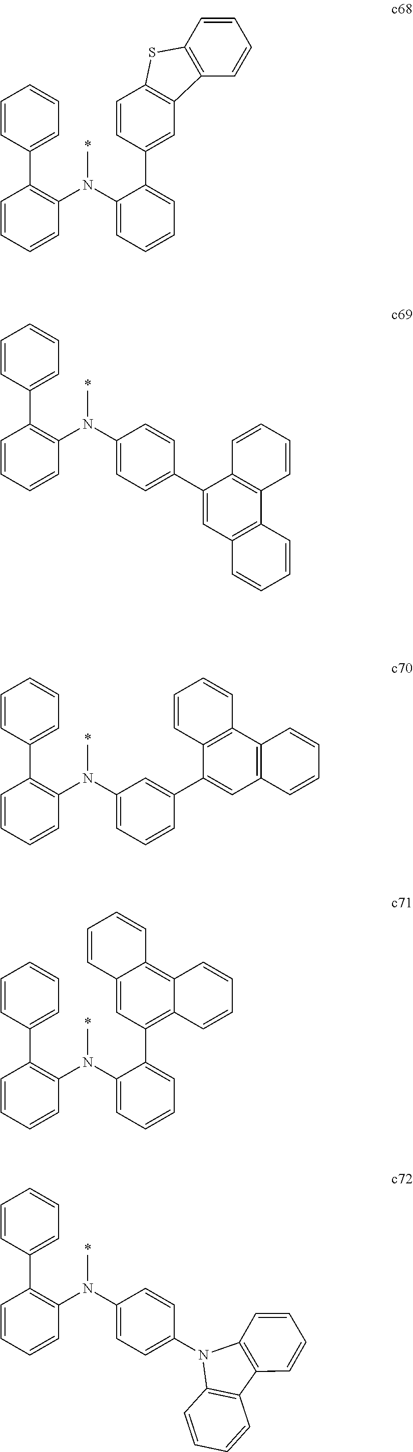 [SMILES: CN(c1ccccc1-c1ccccc1)c1ccccc1-c1ccc2sc3ccccc3c2c1 ; CN(c1ccc(-c2cc3ccccc3c3ccccc23)cc1)c1ccccc1-c1ccccc1 ; CN(c1cccc(-c2cc3ccccc3c3ccccc23)c1)c1ccccc1-c1ccccc1 ; CN(c1ccccc1-c1ccccc1)c1ccccc1-c1cc2ccccc2c2ccccc12 ; CN(c1ccc(-n2c3ccccc3c3ccccc32)cc1)c1ccccc1-c1ccccc1]

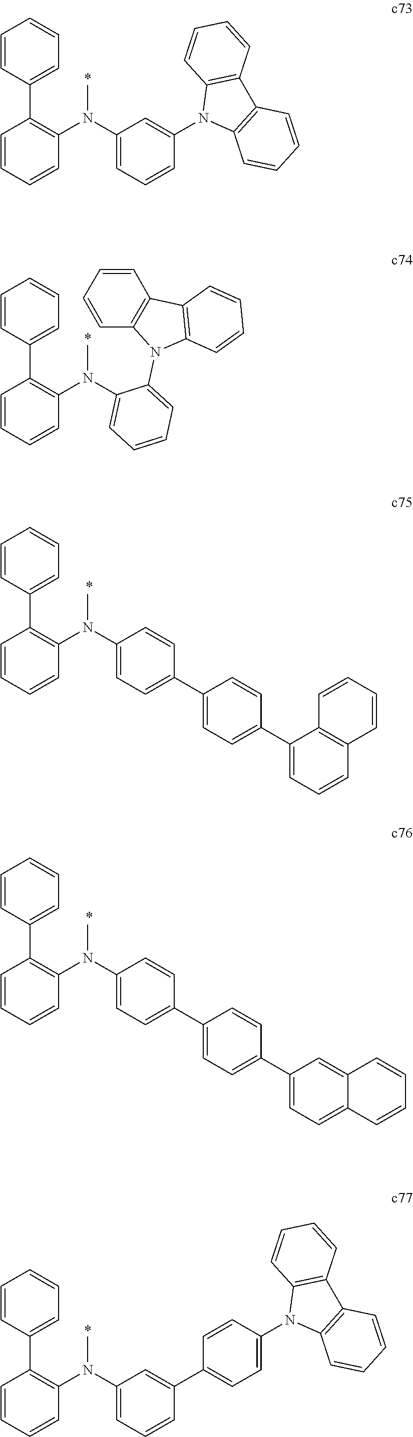 [SMILES: CN(c1cccc(-n2c3ccccc3c3ccccc32)c1)c1ccccc1-c1ccccc1 ; CN(c1ccccc1-c1ccccc1)c1ccccc1-n1c2ccccc2c2ccccc21 ; CN(c1ccc(-c2ccc(-c3cccc4ccccc34)cc2)cc1)c1ccccc1-c1ccccc1 ; CN(c1ccc(-c2ccc(-c3ccc4ccccc4c3)cc2)cc1)c1ccccc1-c1ccccc1 ; CN(c1cccc(-c2ccc(-n3c4ccccc4c4ccccc43)cc2)c1)c1ccccc1-c1ccccc1]

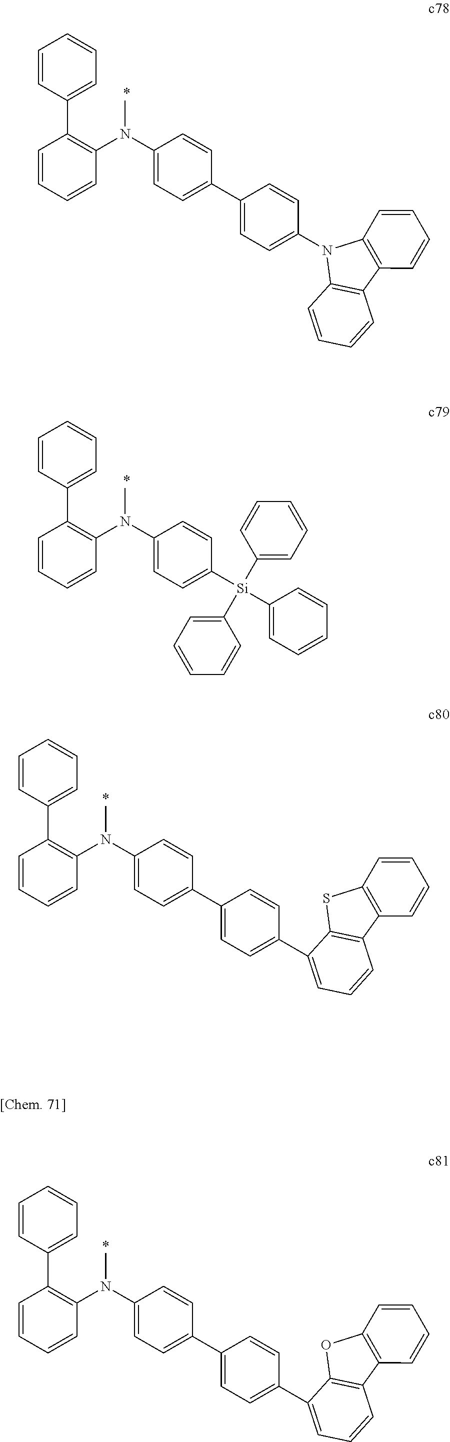 [SMILES: CN(c1ccc(-c2ccc(-n3c4ccccc4c4ccccc43)cc2)cc1)c1ccccc1-c1ccccc1 ; CN(c1ccc([Si](c2ccccc2)(c2ccccc2)c2ccccc2)cc1)c1ccccc1-c1ccccc1 ; CN(c1ccc(-c2ccc(-c3cccc4c3sc3ccccc34)cc2)cc1)c1ccccc1-c1ccccc1 ; CN(c1ccc(-c2ccc(-c3cccc4c3oc3ccccc34)cc2)cc1)c1ccccc1-c1ccccc1]

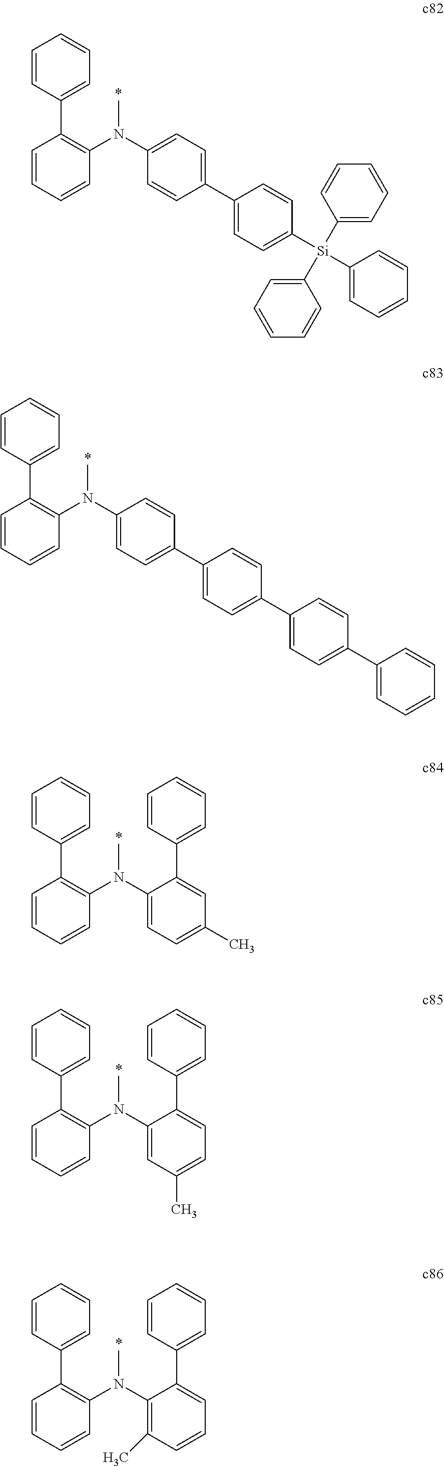 [SMILES: CN(c1ccc(-c2ccc([Si](c3ccccc3)(c3ccccc3)c3ccccc3)cc2)cc1)c1ccccc1-c1ccccc1 ; CN(c1ccc(-c2ccc(-c3ccc(-c4ccccc4)cc3)cc2)cc1)c1ccccc1-c1ccccc1 ; Cc1ccc(N(C)c2ccccc2-c2ccccc2)c(-c2ccccc2)c1 ; Cc1ccc(-c2ccccc2)c(N(C)c2ccccc2-c2ccccc2)c1 ; Cc1cccc(-c2ccccc2)c1N(C)c1ccccc1-c1ccccc1]

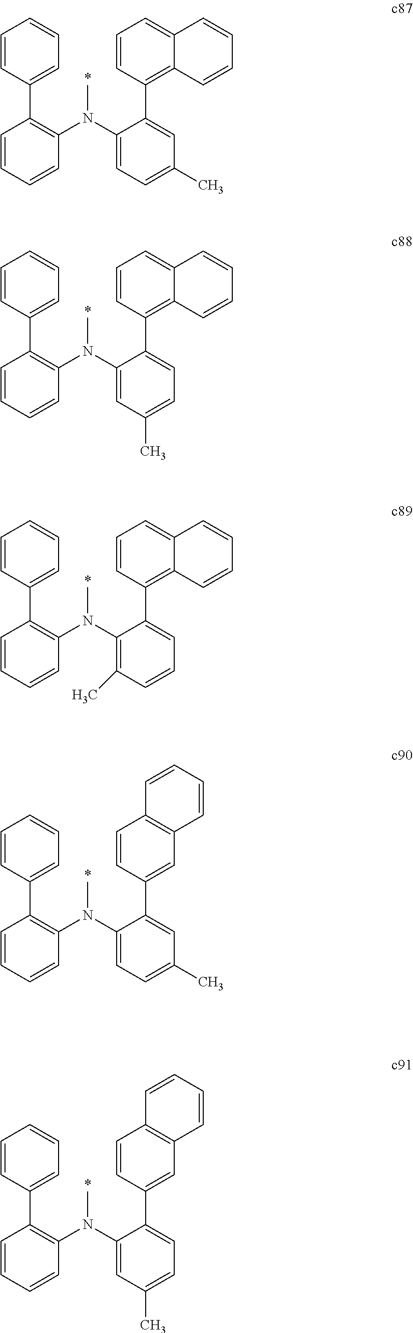 [SMILES: Cc1ccc(N(C)c2ccccc2-c2ccccc2)c(-c2cccc3ccccc23)c1 ; Cc1ccc(-c2cccc3ccccc23)c(N(C)c2ccccc2-c2ccccc2)c1 ; Cc1cccc(-c2cccc3ccccc23)c1N(C)c1ccccc1-c1ccccc1 ; Cc1ccc(N(C)c2ccccc2-c2ccccc2)c(-c2ccc3ccccc3c2)c1 ; Cc1ccc(-c2ccc3ccccc3c2)c(N(C)c2ccccc2-c2ccccc2)c1]

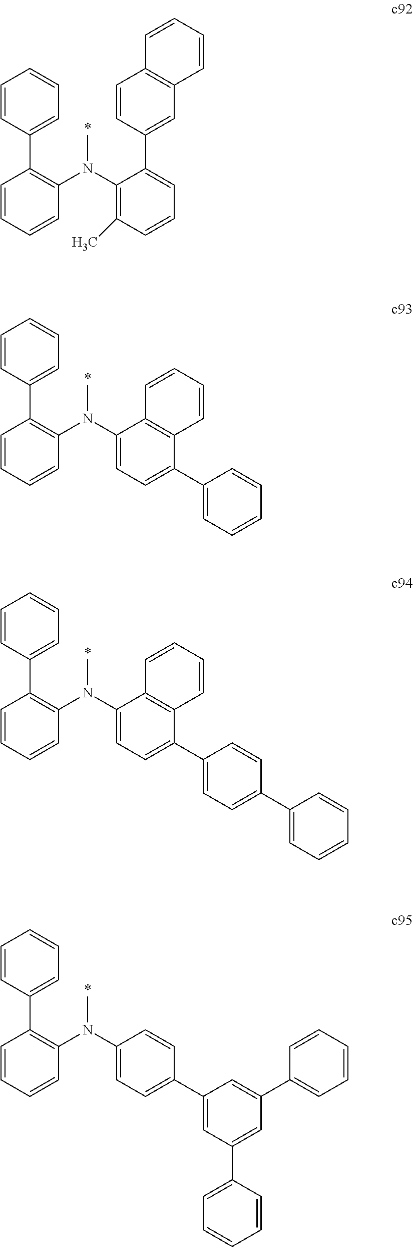 [SMILES: Cc1cccc(-c2ccc3ccccc3c2)c1N(C)c1ccccc1-c1ccccc1 ; CN(c1ccccc1-c1ccccc1)c1ccc(-c2ccccc2)c2ccccc12 ; CN(c1ccccc1-c1ccccc1)c1ccc(-c2ccc(-c3ccccc3)cc2)c2ccccc12 ; CN(c1ccc(-c2cc(-c3ccccc3)cc(-c3ccccc3)c2)cc1)c1ccccc1-c1ccccc1]

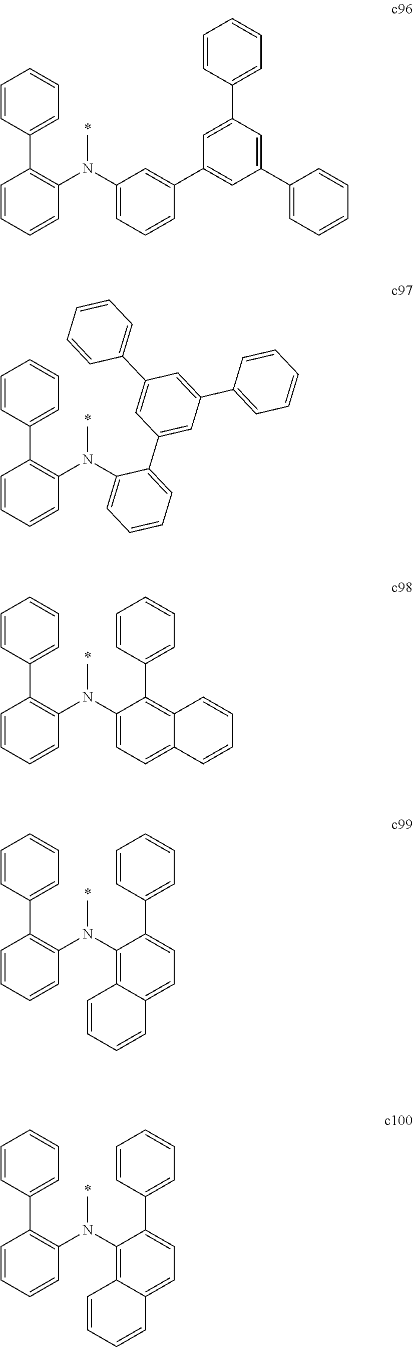 [SMILES: CN(c1cccc(-c2cc(-c3ccccc3)cc(-c3ccccc3)c2)c1)c1ccccc1-c1ccccc1 ; CN(c1ccccc1-c1ccccc1)c1ccccc1-c1cc(-c2ccccc2)cc(-c2ccccc2)c1 ; CN(c1ccccc1-c1ccccc1)c1ccc2ccccc2c1-c1ccccc1 ; CN(c1ccccc1-c1ccccc1)c1c(-c2ccccc2)ccc2ccccc12 ; CN(c1ccccc1-c1ccccc1)c1c(-c2ccccc2)ccc2ccccc12]

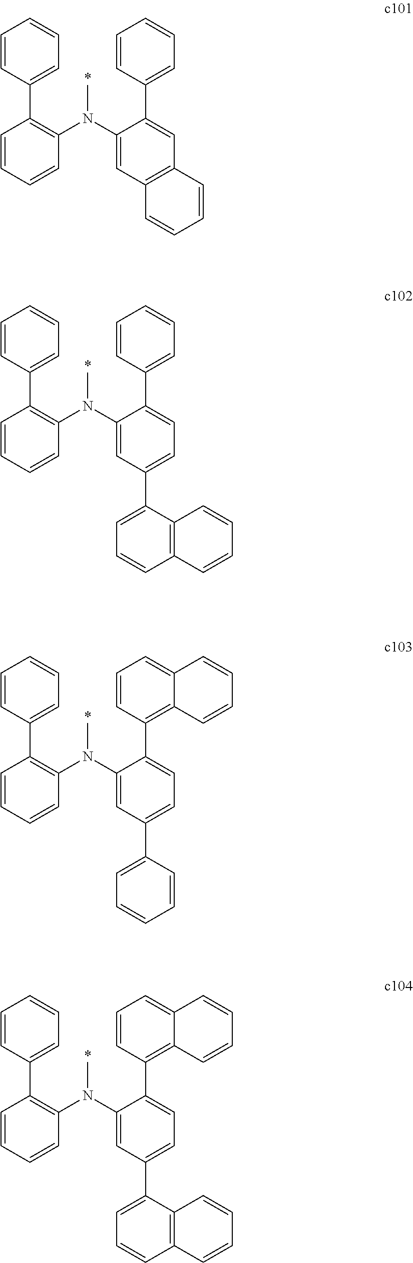 [SMILES: CN(c1ccccc1-c1ccccc1)c1cc2ccccc2cc1-c1ccccc1 ; CN(c1ccccc1-c1ccccc1)c1cc(-c2cccc3ccccc23)ccc1-c1ccccc1 ; CN(c1ccccc1-c1ccccc1)c1cc(-c2ccccc2)ccc1-c1cccc2ccccc12 ; CN(c1ccccc1-c1ccccc1)c1cc(-c2cccc3ccccc23)ccc1-c1cccc2ccccc12]

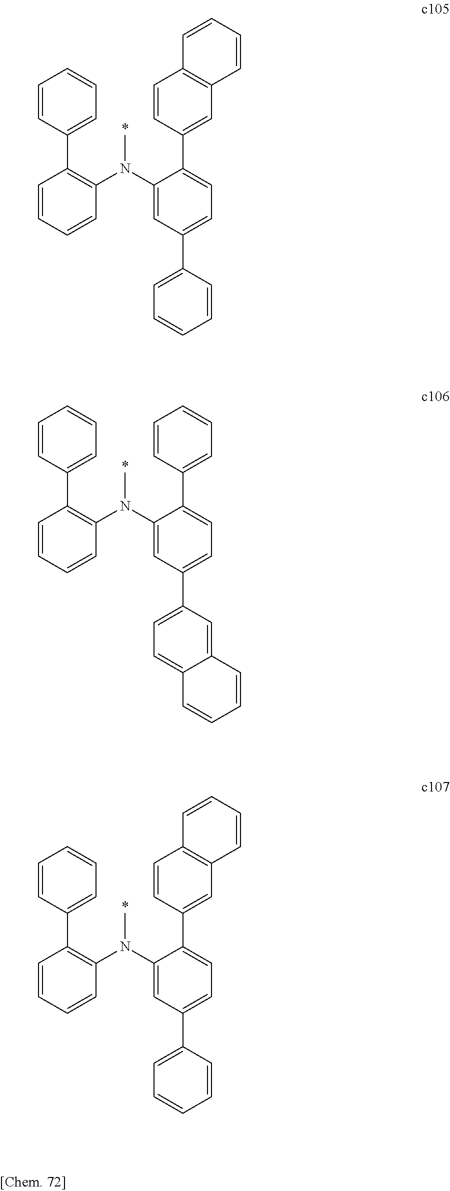 [SMILES: CN(c1ccccc1-c1ccccc1)c1cc(-c2ccccc2)ccc1-c1ccc2ccccc2c1 ; CN(c1ccccc1-c1ccccc1)c1cc(-c2ccc3ccccc3c2)ccc1-c1ccccc1 ; CN(c1ccccc1-c1ccccc1)c1cc(-c2ccccc2)ccc1-c1ccc2ccccc2c1]

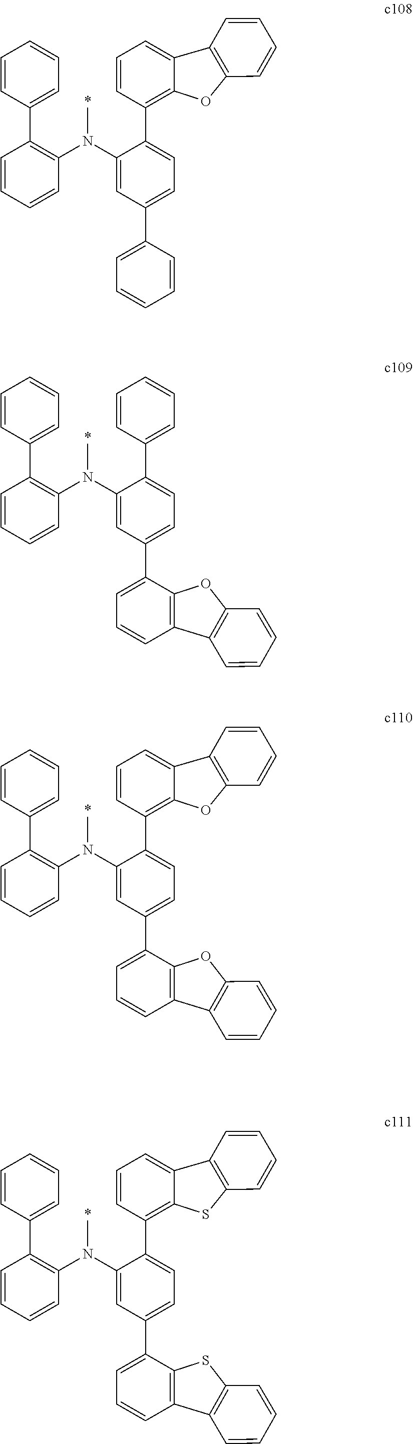 [SMILES: CN(c1ccccc1-c1ccccc1)c1cc(-c2ccccc2)ccc1-c1cccc2c1oc1ccccc12 ; CN(c1ccccc1-c1ccccc1)c1cc(-c2cccc3c2oc2ccccc23)ccc1-c1ccccc1 ; CN(c1ccccc1-c1ccccc1)c1cc(-c2cccc3c2oc2ccccc23)ccc1-c1cccc2c1oc1ccccc12 ; CN(c1ccccc1-c1ccccc1)c1cc(-c2cccc3c2sc2ccccc23)ccc1-c1cccc2c1sc1ccccc12]

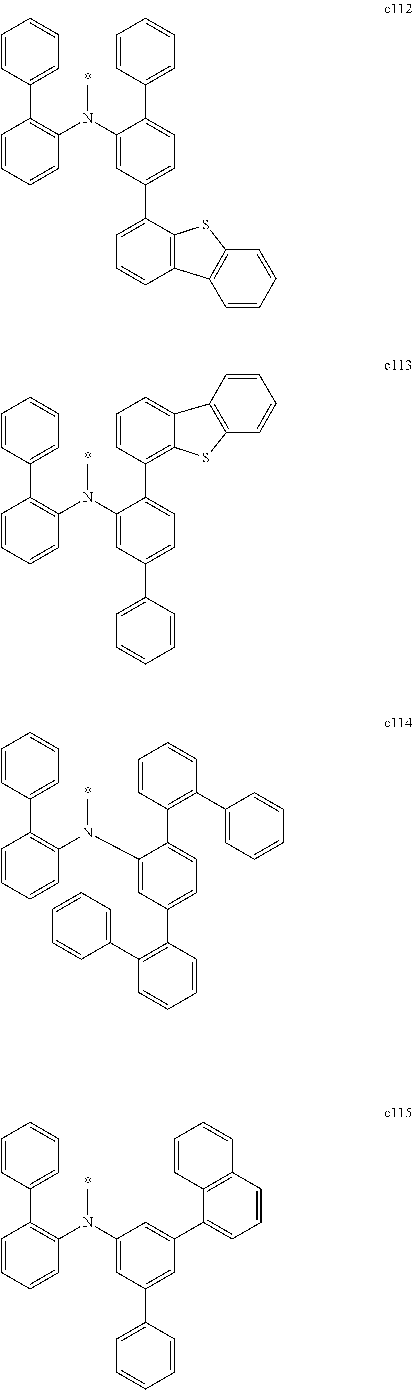 [SMILES: CN(c1ccccc1-c1ccccc1)c1cc(-c2cccc3c2sc2ccccc23)ccc1-c1ccccc1 ; CN(c1ccccc1-c1ccccc1)c1cc(-c2ccccc2)ccc1-c1cccc2c1sc1ccccc12 ; CN(c1ccccc1-c1ccccc1)c1cc(-c2ccccc2-c2ccccc2)ccc1-c1ccccc1-c1ccccc1 ; CN(c1cc(-c2ccccc2)cc(-c2cccc3ccccc23)c1)c1ccccc1-c1ccccc1]

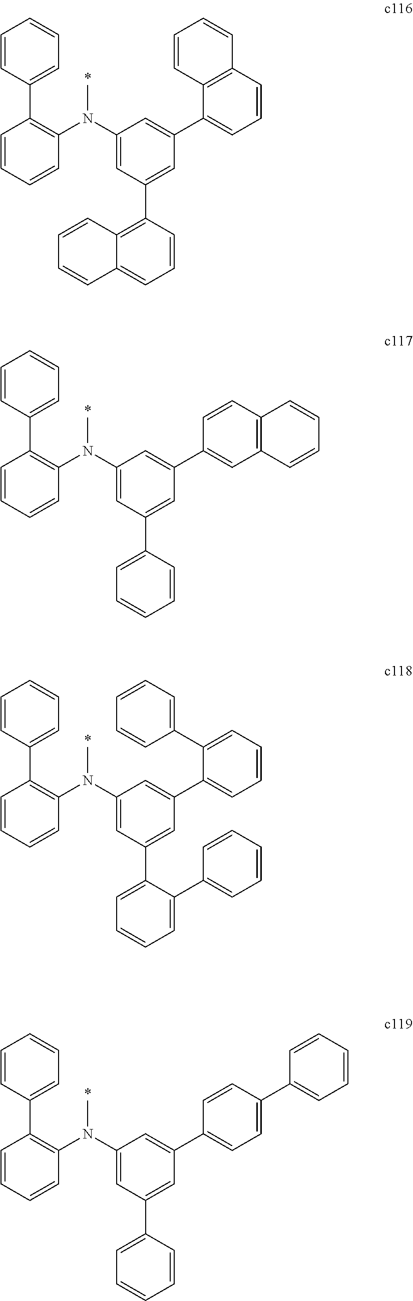 [SMILES: CN(c1cc(-c2cccc3ccccc23)cc(-c2cccc3ccccc23)c1)c1ccccc1-c1ccccc1 ; CN(c1cc(-c2ccccc2)cc(-c2ccc3ccccc3c2)c1)c1ccccc1-c1ccccc1 ; CN(c1cc(-c2ccccc2-c2ccccc2)cc(-c2ccccc2-c2ccccc2)c1)c1ccccc1-c1ccccc1 ; CN(c1cc(-c2ccccc2)cc(-c2ccc(-c3ccccc3)cc2)c1)c1ccccc1-c1ccccc1]

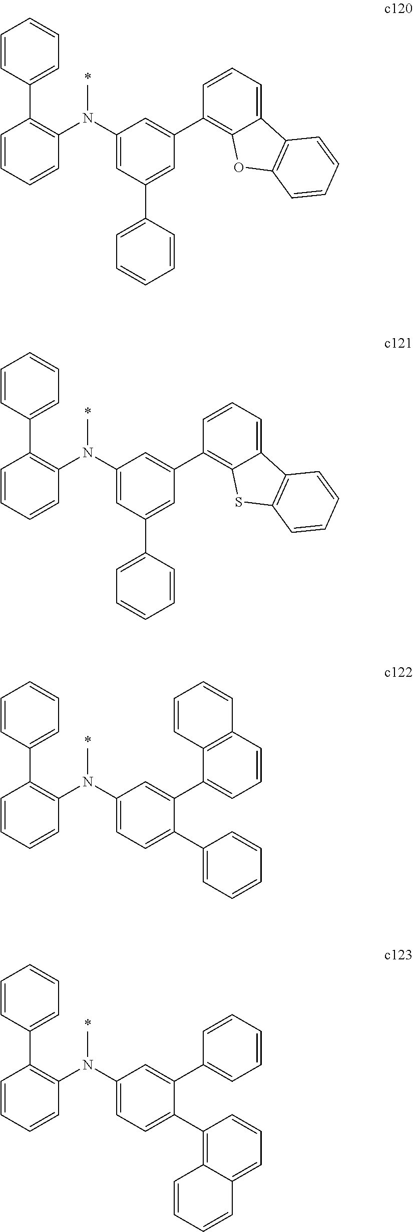 [SMILES: CN(c1cc(-c2ccccc2)cc(-c2cccc3c2oc2ccccc23)c1)c1ccccc1-c1ccccc1 ; CN(c1cc(-c2ccccc2)cc(-c2cccc3c2sc2ccccc23)c1)c1ccccc1-c1ccccc1 ; CN(c1ccc(-c2ccccc2)c(-c2cccc3ccccc23)c1)c1ccccc1-c1ccccc1 ; CN(c1ccc(-c2cccc3ccccc23)c(-c2ccccc2)c1)c1ccccc1-c1ccccc1]

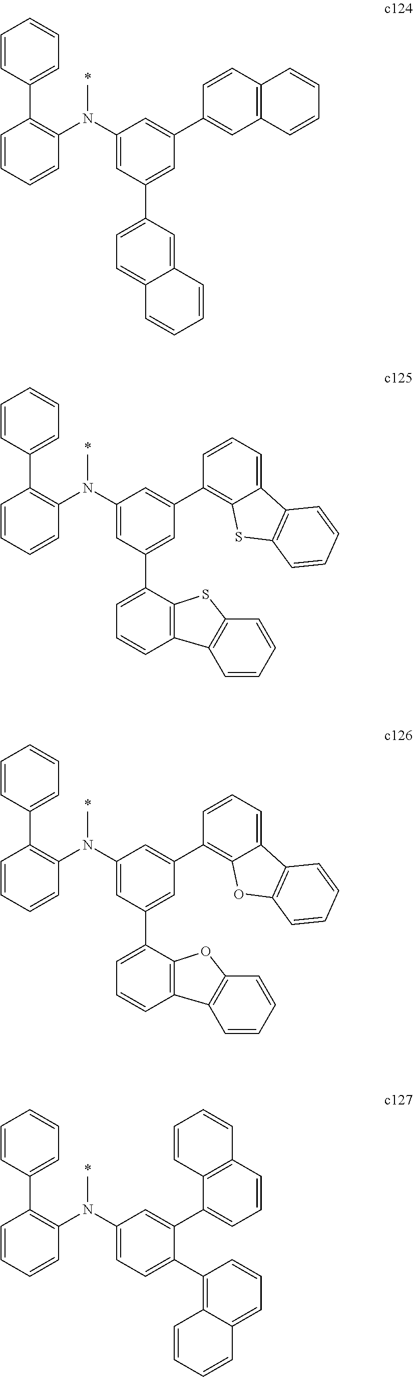 [SMILES: CN(c1cc(-c2ccc3ccccc3c2)cc(-c2ccc3ccccc3c2)c1)c1ccccc1-c1ccccc1 ; CN(c1cc(-c2cccc3c2sc2ccccc23)cc(-c2cccc3c2sc2ccccc23)c1)c1ccccc1-c1ccccc1 ; CN(c1cc(-c2cccc3c2oc2ccccc23)cc(-c2cccc3c2oc2ccccc23)c1)c1ccccc1-c1ccccc1 ; CN(c1ccc(-c2cccc3ccccc23)c(-c2cccc3ccccc23)c1)c1ccccc1-c1ccccc1]

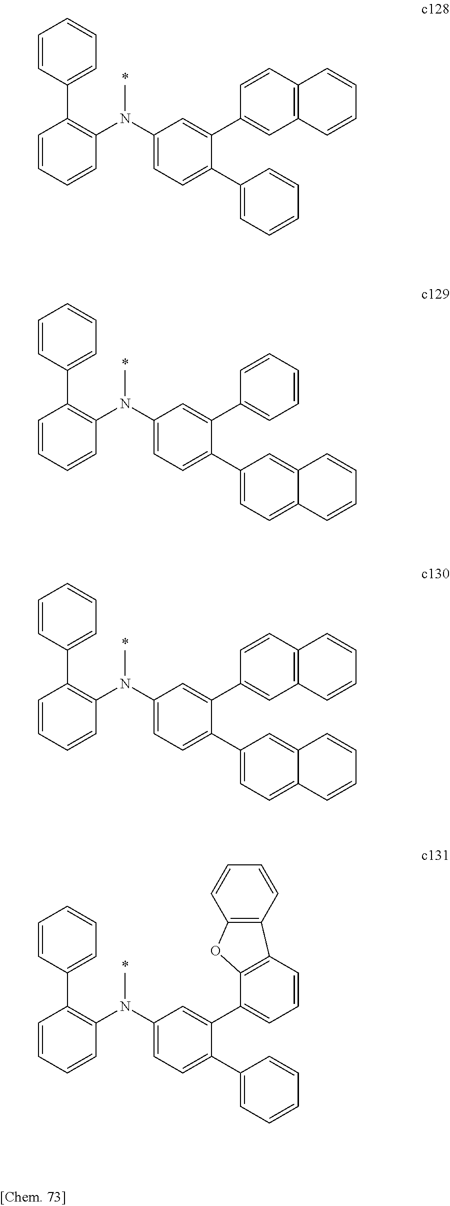 [SMILES: CN(c1ccc(-c2ccccc2)c(-c2ccc3ccccc3c2)c1)c1ccccc1-c1ccccc1 ; CN(c1ccc(-c2ccc3ccccc3c2)c(-c2ccccc2)c1)c1ccccc1-c1ccccc1 ; CN(c1ccc(-c2ccc3ccccc3c2)c(-c2ccc3ccccc3c2)c1)c1ccccc1-c1ccccc1 ; CN(c1ccc(-c2ccccc2)c(-c2cccc3c2oc2ccccc23)c1)c1ccccc1-c1ccccc1]

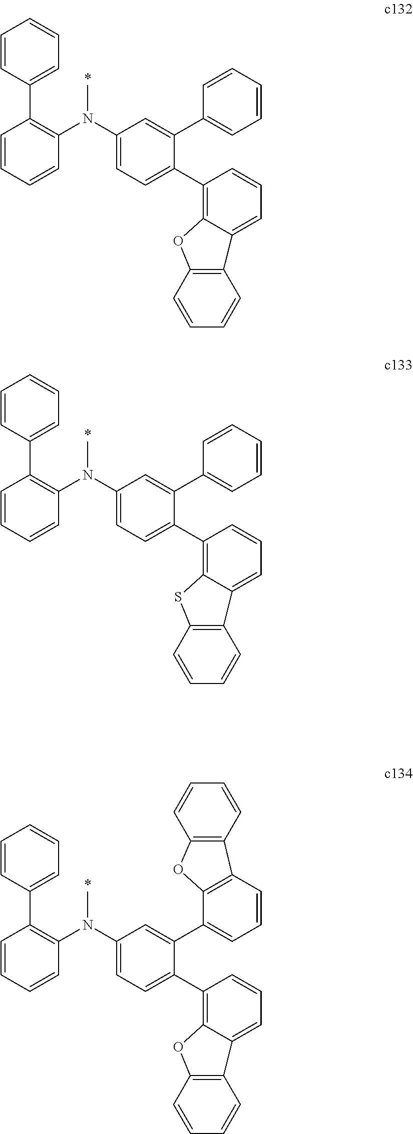 [SMILES: CN(c1ccc(-c2cccc3c2oc2ccccc23)c(-c2ccccc2)c1)c1ccccc1-c1ccccc1 ; CN(c1ccc(-c2cccc3c2sc2ccccc23)c(-c2ccccc2)c1)c1ccccc1-c1ccccc1 ; CN(c1ccc(-c2cccc3c2oc2ccccc23)c(-c2cccc3c2oc2ccccc23)c1)c1ccccc1-c1ccccc1]

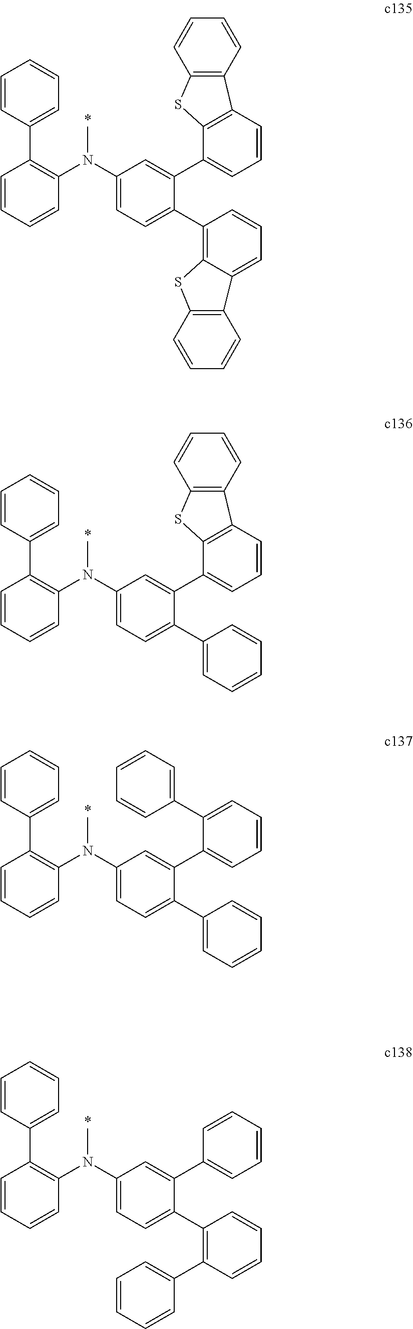 [SMILES: CN(c1ccc(-c2cccc3c2sc2ccccc23)c(-c2cccc3c2sc2ccccc23)c1)c1ccccc1-c1ccccc1 ; CN(c1ccc(-c2ccccc2)c(-c2cccc3c2sc2ccccc23)c1)c1ccccc1-c1ccccc1 ; CN(c1ccc(-c2ccccc2)c(-c2ccccc2-c2ccccc2)c1)c1ccccc1-c1ccccc1 ; CN(c1ccc(-c2ccccc2-c2ccccc2)c(-c2ccccc2)c1)c1ccccc1-c1ccccc1]

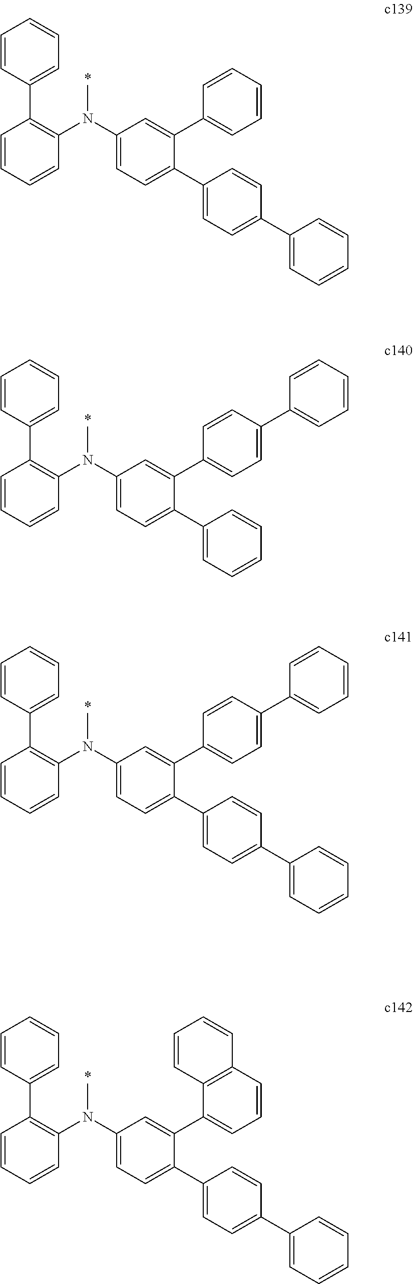 [SMILES: CN(c1ccc(-c2ccc(-c3ccccc3)cc2)c(-c2ccccc2)c1)c1ccccc1-c1ccccc1 ; CN(c1ccc(-c2ccccc2)c(-c2ccc(-c3ccccc3)cc2)c1)c1ccccc1-c1ccccc1 ; CN(c1ccc(-c2ccc(-c3ccccc3)cc2)c(-c2ccc(-c3ccccc3)cc2)c1)c1ccccc1-c1ccccc1 ; CN(c1ccc(-c2ccc(-c3ccccc3)cc2)c(-c2cccc3ccccc23)c1)c1ccccc1-c1ccccc1]

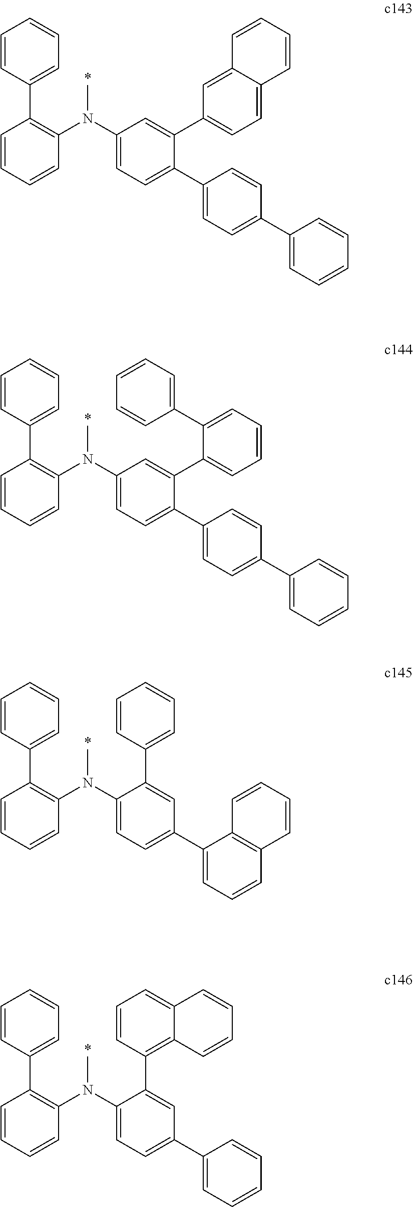 [SMILES: CN(c1ccc(-c2ccc(-c3ccccc3)cc2)c(-c2ccc3ccccc3c2)c1)c1ccccc1-c1ccccc1 ; CN(c1ccc(-c2ccc(-c3ccccc3)cc2)c(-c2ccccc2-c2ccccc2)c1)c1ccccc1-c1ccccc1 ; CN(c1ccccc1-c1ccccc1)c1ccc(-c2cccc3ccccc23)cc1-c1ccccc1 ; CN(c1ccccc1-c1ccccc1)c1ccc(-c2ccccc2)cc1-c1cccc2ccccc12]

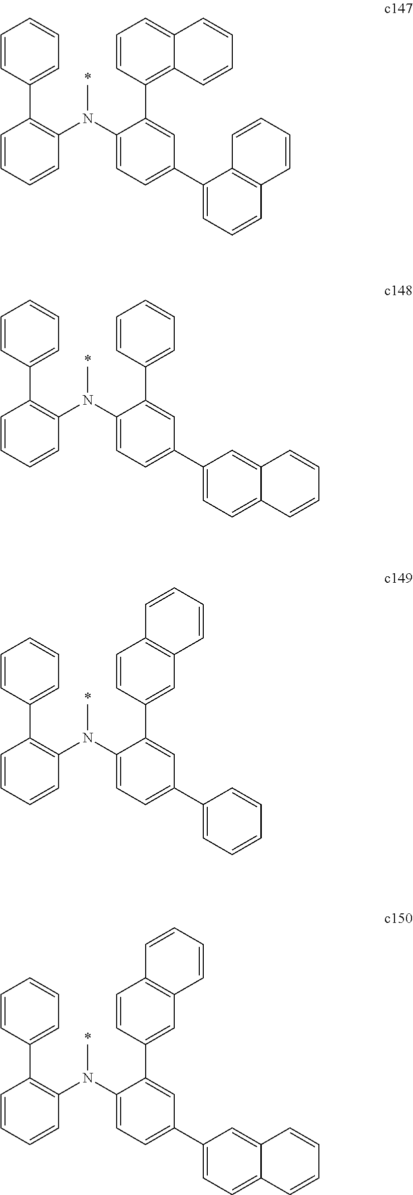 [SMILES: CN(c1ccccc1-c1ccccc1)c1ccc(-c2cccc3ccccc23)cc1-c1cccc2ccccc12 ; CN(c1ccccc1-c1ccccc1)c1ccc(-c2ccc3ccccc3c2)cc1-c1ccccc1 ; CN(c1ccccc1-c1ccccc1)c1ccc(-c2ccccc2)cc1-c1ccc2ccccc2c1 ; CN(c1ccccc1-c1ccccc1)c1ccc(-c2ccc3ccccc3c2)cc1-c1ccc2ccccc2c1]

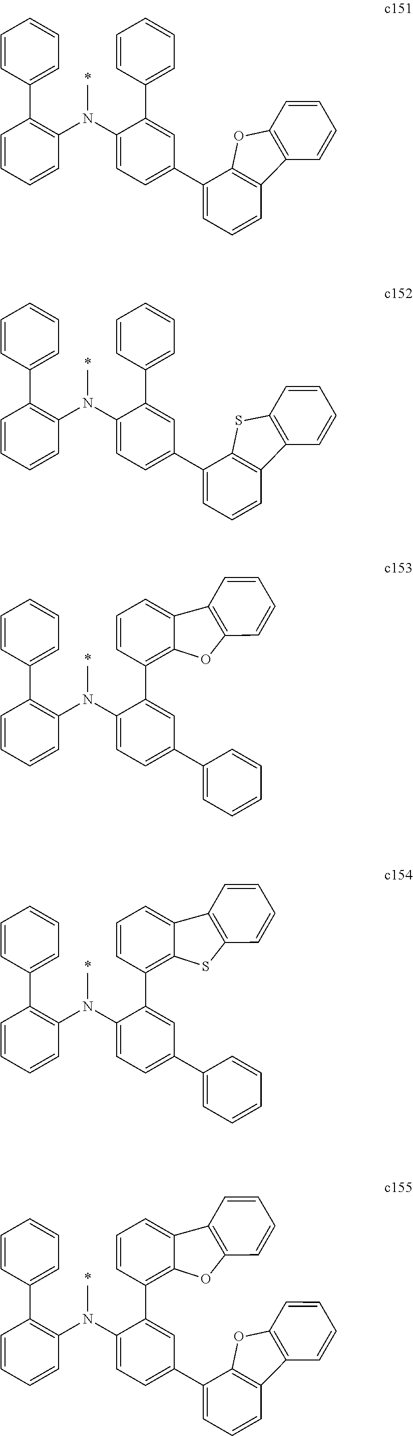 [SMILES: CN(c1ccccc1-c1ccccc1)c1ccc(-c2cccc3c2oc2ccccc23)cc1-c1ccccc1 ; CN(c1ccccc1-c1ccccc1)c1ccc(-c2cccc3c2sc2ccccc23)cc1-c1ccccc1 ; CN(c1ccccc1-c1ccccc1)c1ccc(-c2ccccc2)cc1-c1cccc2c1oc1ccccc12 ; CN(c1ccccc1-c1ccccc1)c1ccc(-c2ccccc2)cc1-c1cccc2c1sc1ccccc12 ; CN(c1ccccc1-c1ccccc1)c1ccc(-c2cccc3c2oc2ccccc23)cc1-c1cccc2c1oc1ccccc12]

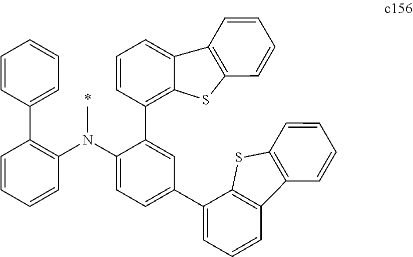 [SMILES: CN(c1ccccc1-c1ccccc1)c1ccc(-c2cccc3c2sc2ccccc23)cc1-c1cccc2c1sc1ccccc12]

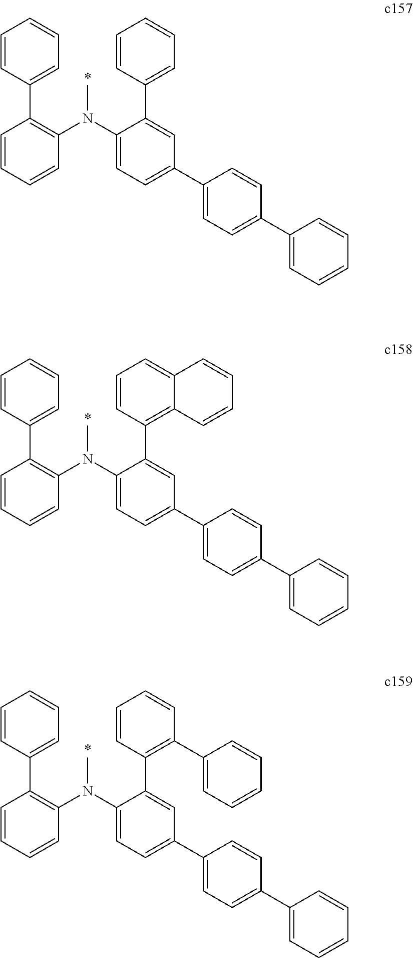 [SMILES: CN(c1ccccc1-c1ccccc1)c1ccc(-c2ccc(-c3ccccc3)cc2)cc1-c1ccccc1 ; CN(c1ccccc1-c1ccccc1)c1ccc(-c2ccc(-c3ccccc3)cc2)cc1-c1cccc2ccccc12 ; CN(c1ccccc1-c1ccccc1)c1ccc(-c2ccc(-c3ccccc3)cc2)cc1-c1ccccc1-c1ccccc1]

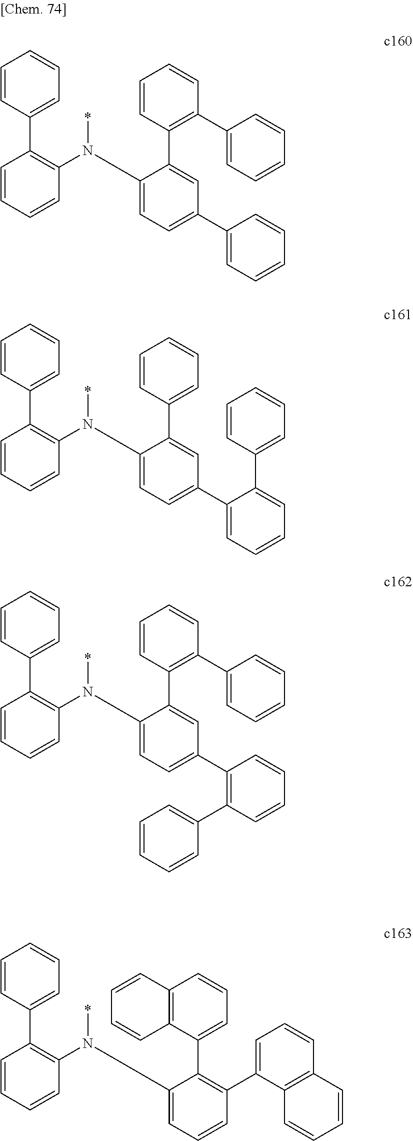 [SMILES: *N(c1ccccc1-c1ccccc1)c1ccc(-c2ccccc2)cc1-c1ccccc1-c1ccccc1 ; *N(c1ccccc1-c1ccccc1)c1ccc(-c2ccccc2-c2ccccc2)cc1-c1ccccc1 ; *N(c1ccccc1-c1ccccc1)c1ccc(-c2ccccc2-c2ccccc2)cc1-c1ccccc1-c1ccccc1 ; *N(c1ccccc1-c1ccccc1)c1cccc(-c2cccc3ccccc23)c1-c1cccc2ccccc12]

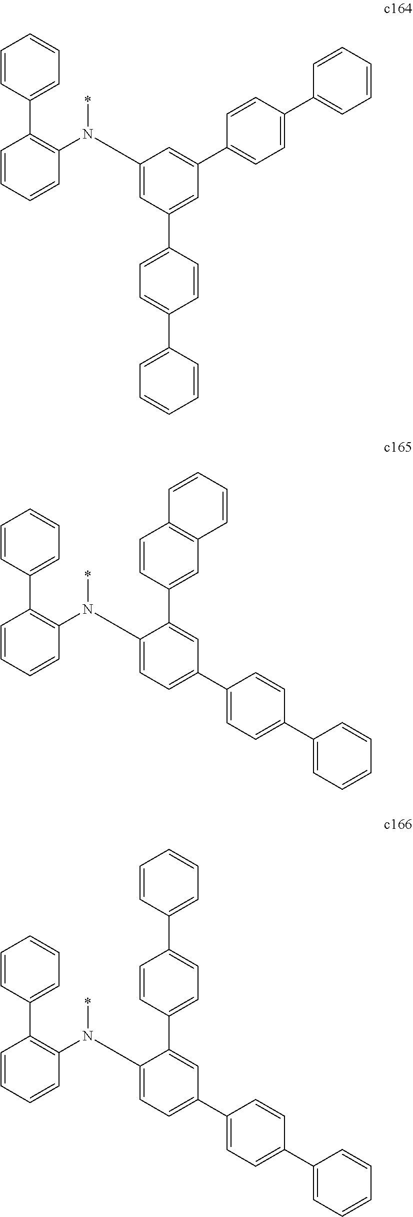 [SMILES: *N(c1cc(-c2ccc(-c3ccccc3)cc2)cc(-c2ccc(-c3ccccc3)cc2)c1)c1ccccc1-c1ccccc1 ; *N(c1ccccc1-c1ccccc1)c1ccc(-c2ccc(-c3ccccc3)cc2)cc1-c1ccc2ccccc2c1 ; *N(c1ccccc1-c1ccccc1)c1ccc(-c2ccc(-c3ccccc3)cc2)cc1-c1ccc(-c2ccccc2)cc1]

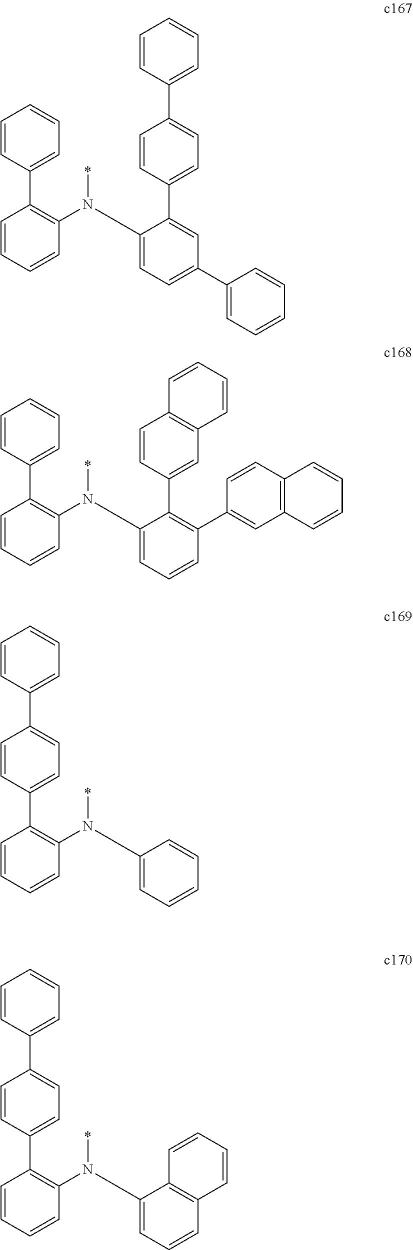 [SMILES: *N(c1ccccc1-c1ccccc1)c1ccc(-c2ccccc2)cc1-c1ccc(-c2ccccc2)cc1 ; *N(c1ccccc1-c1ccccc1)c1cccc(-c2ccc3ccccc3c2)c1-c1ccc2ccccc2c1 ; *N(c1ccccc1)c1ccccc1-c1ccc(-c2ccccc2)cc1 ; *N(c1ccccc1-c1ccc(-c2ccccc2)cc1)c1cccc2ccccc12]

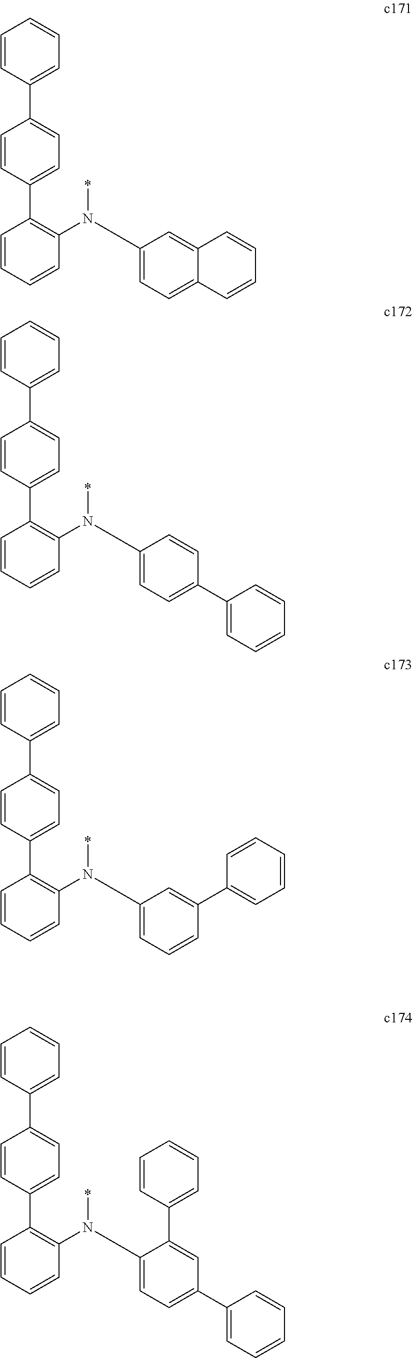

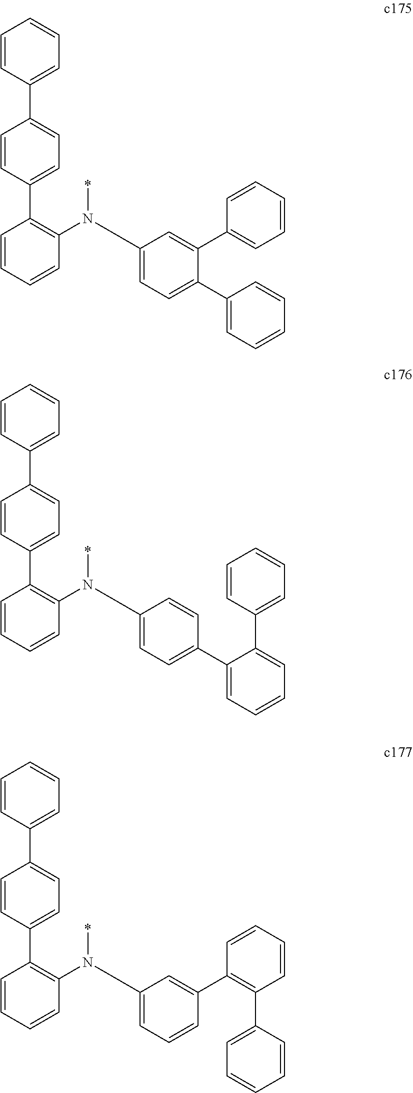 [SMILES: *N(c1ccc(-c2ccccc2)c(-c2ccccc2)c1)c1ccccc1-c1ccc(-c2ccccc2)cc1 ; *N(c1ccc(-c2ccccc2-c2ccccc2)cc1)c1ccccc1-c1ccc(-c2ccccc2)cc1 ; *N(c1cccc(-c2ccccc2-c2ccccc2)c1)c1ccccc1-c1ccc(-c2ccccc2)cc1]

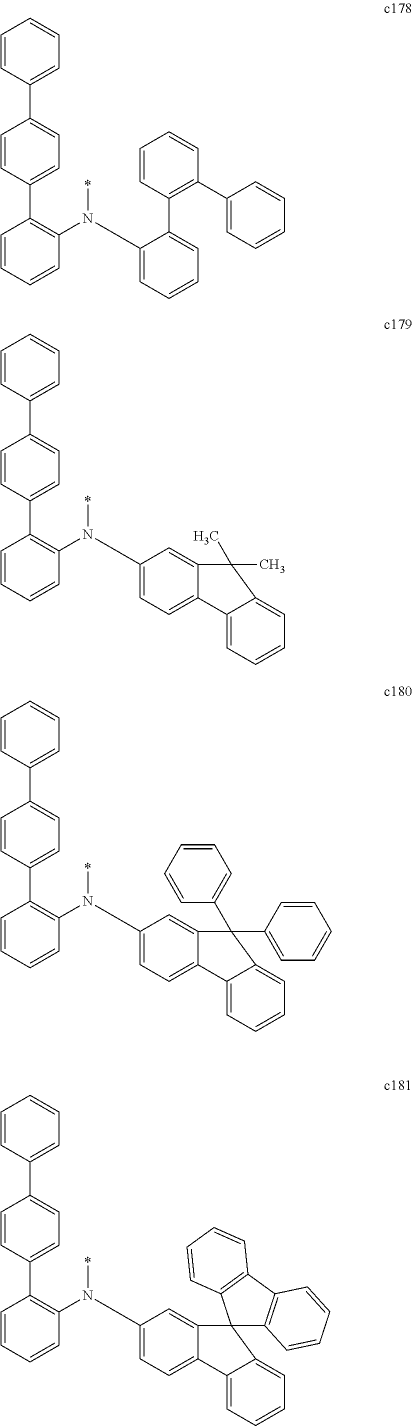 [SMILES: *N(c1ccccc1-c1ccc(-c2ccccc2)cc1)c1ccccc1-c1ccccc1-c1ccccc1 ; *N(c1ccc2c(c1)C(C)(C)c1ccccc1-2)c1ccccc1-c1ccc(-c2ccccc2)cc1 ; *N(c1ccc2c(c1)C(c1ccccc1)(c1ccccc1)c1ccccc1-2)c1ccccc1-c1ccc(-c2ccccc2)cc1 ; *N(c1ccc2c(c1)C1(c3ccccc3-c3ccccc31)c1ccccc1-2)c1ccccc1-c1ccc(-c2ccccc2)cc1]

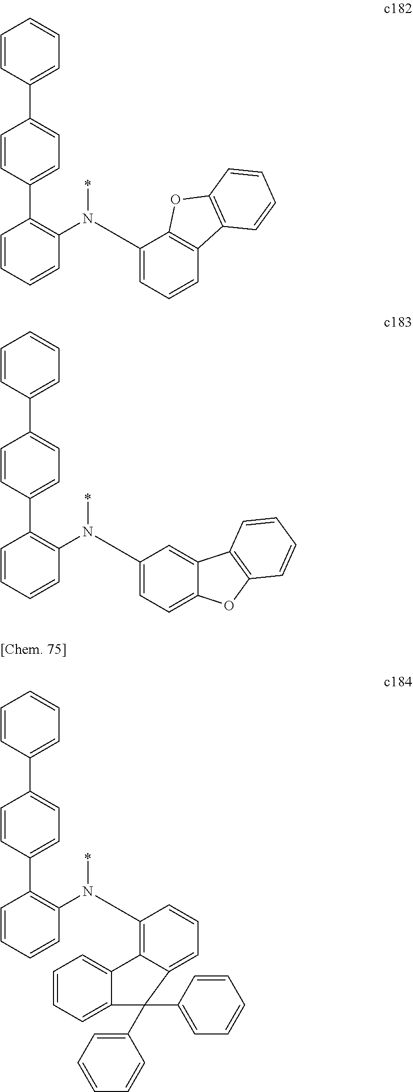 [SMILES: *N(c1ccccc1-c1ccc(-c2ccccc2)cc1)c1cccc2c1oc1ccccc12 ; *N(c1ccc2oc3ccccc3c2c1)c1ccccc1-c1ccc(-c2ccccc2)cc1 ; *N(c1ccccc1-c1ccc(-c2ccccc2)cc1)c1cccc2c1-c1ccccc1C2(c1ccccc1)c1ccccc1]

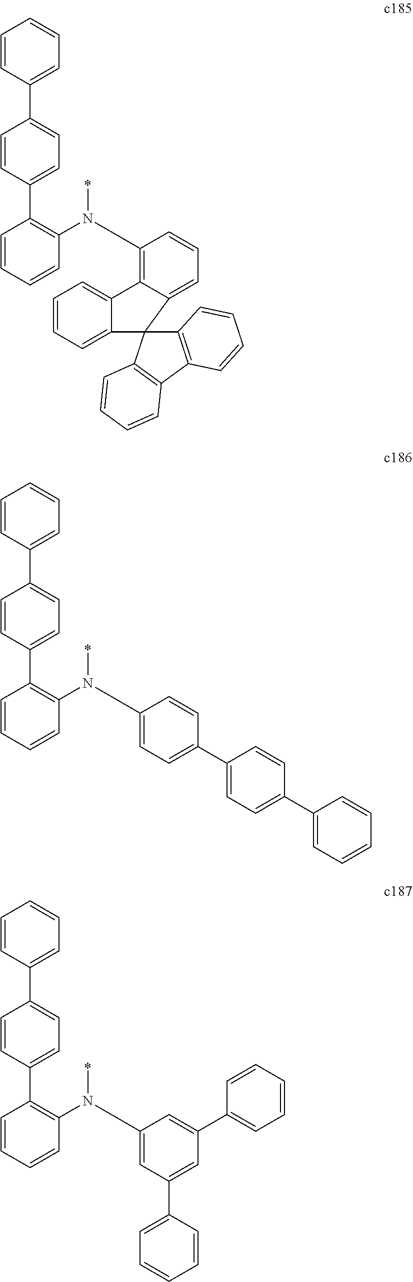 [SMILES: *N(c1ccccc1-c1ccc(-c2ccccc2)cc1)c1cccc2c1-c1ccccc1C21c2ccccc2-c2ccccc21 ; *N(c1ccc(-c2ccc(-c3ccccc3)cc2)cc1)c1ccccc1-c1ccc(-c2ccccc2)cc1 ; *N(c1cc(-c2ccccc2)cc(-c2ccccc2)c1)c1ccccc1-c1ccc(-c2ccccc2)cc1]

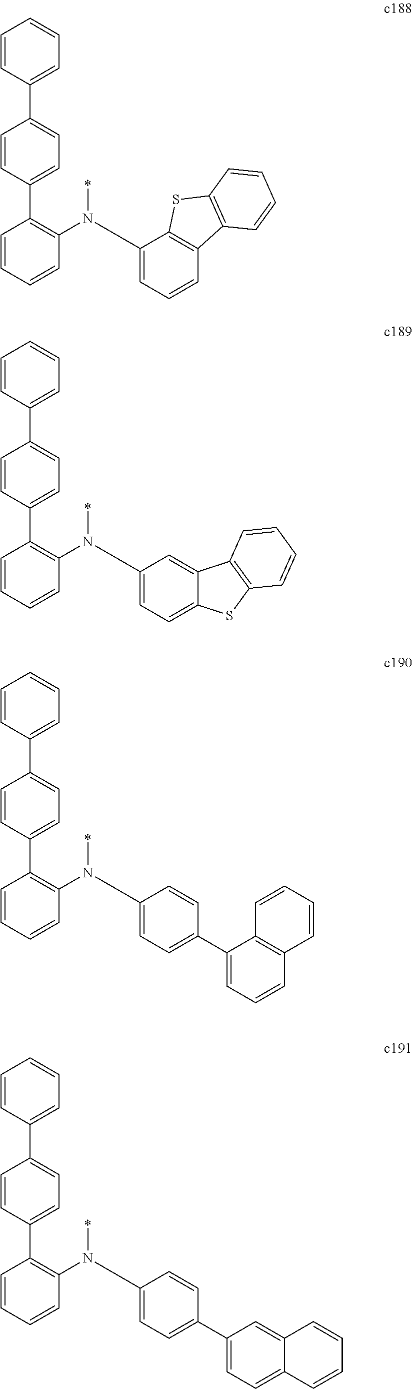 [SMILES: *N(c1ccccc1-c1ccc(-c2ccccc2)cc1)c1cccc2c1sc1ccccc12 ; *N(c1ccc2sc3ccccc3c2c1)c1ccccc1-c1ccc(-c2ccccc2)cc1 ; *N(c1ccc(-c2cccc3ccccc23)cc1)c1ccccc1-c1ccc(-c2ccccc2)cc1 ; *N(c1ccc(-c2ccc3ccccc3c2)cc1)c1ccccc1-c1ccc(-c2ccccc2)cc1]

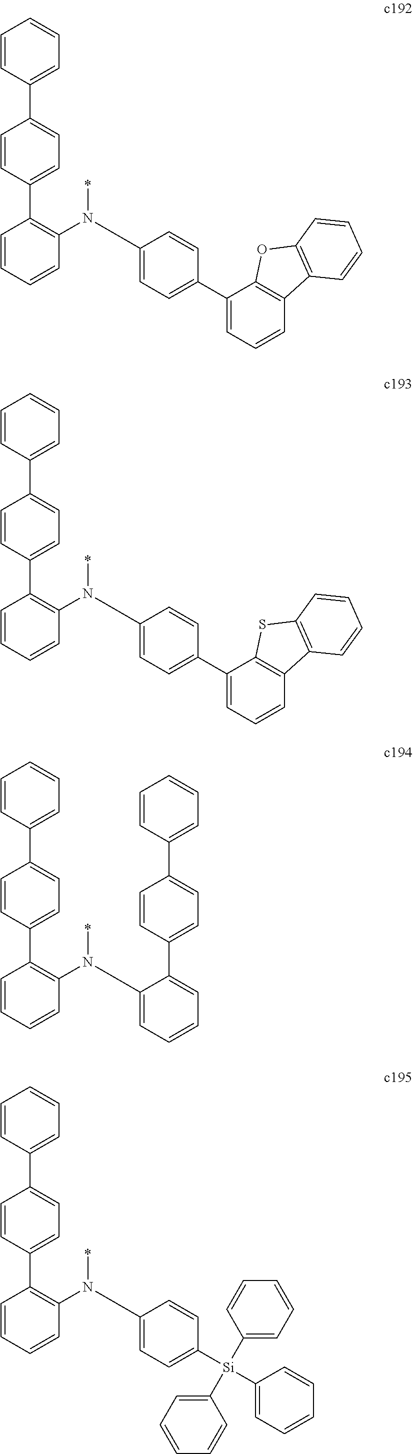 [SMILES: *N(c1ccc(-c2cccc3c2oc2ccccc23)cc1)c1ccccc1-c1ccc(-c2ccccc2)cc1 ; *N(c1ccc(-c2cccc3c2sc2ccccc23)cc1)c1ccccc1-c1ccc(-c2ccccc2)cc1 ; *N(c1ccccc1-c1ccc(-c2ccccc2)cc1)c1ccccc1-c1ccc(-c2ccccc2)cc1 ; *N(c1ccc([Si](c2ccccc2)(c2ccccc2)c2ccccc2)cc1)c1ccccc1-c1ccc(-c2ccccc2)cc1]

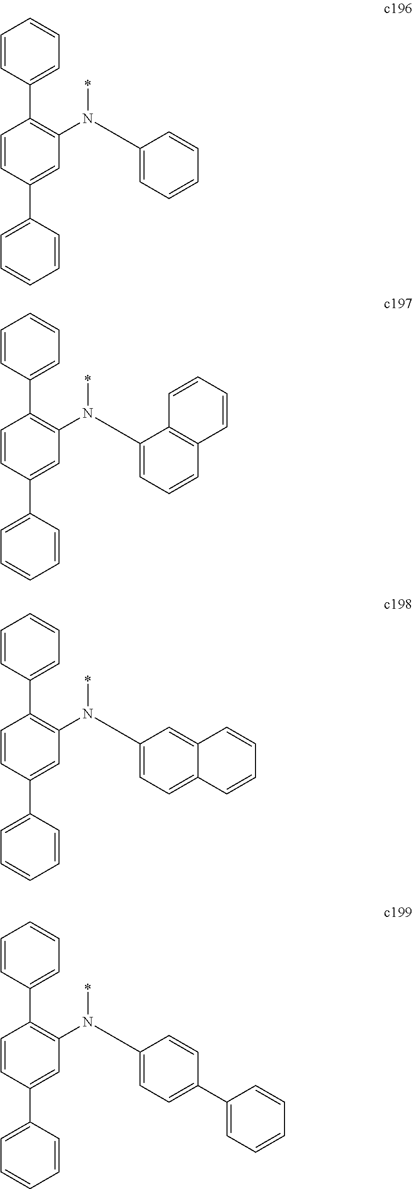

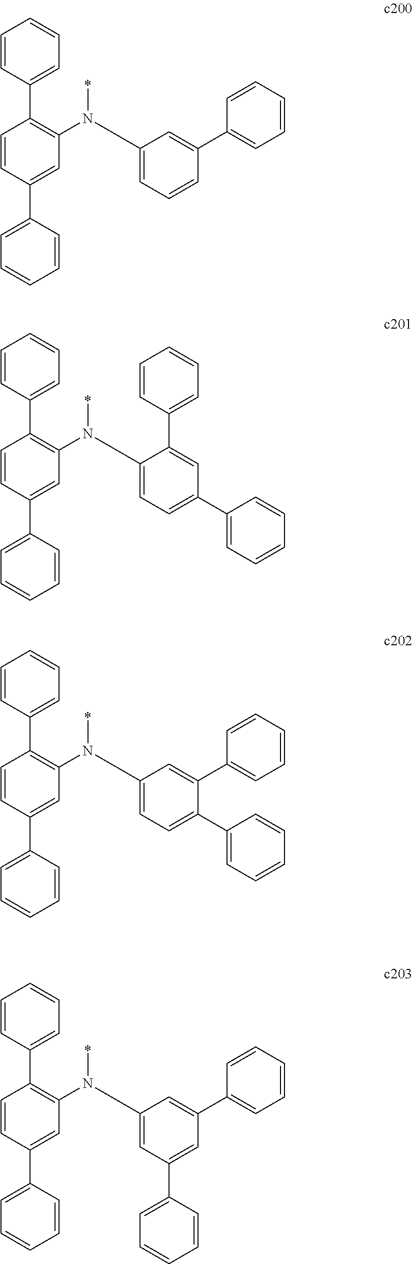 [SMILES: *N(c1cccc(-c2ccccc2)c1)c1cc(-c2ccccc2)ccc1-c1ccccc1 ; *N(c1ccc(-c2ccccc2)cc1-c1ccccc1)c1cc(-c2ccccc2)ccc1-c1ccccc1 ; *N(c1ccc(-c2ccccc2)c(-c2ccccc2)c1)c1cc(-c2ccccc2)ccc1-c1ccccc1 ; *N(c1cc(-c2ccccc2)cc(-c2ccccc2)c1)c1cc(-c2ccccc2)ccc1-c1ccccc1]

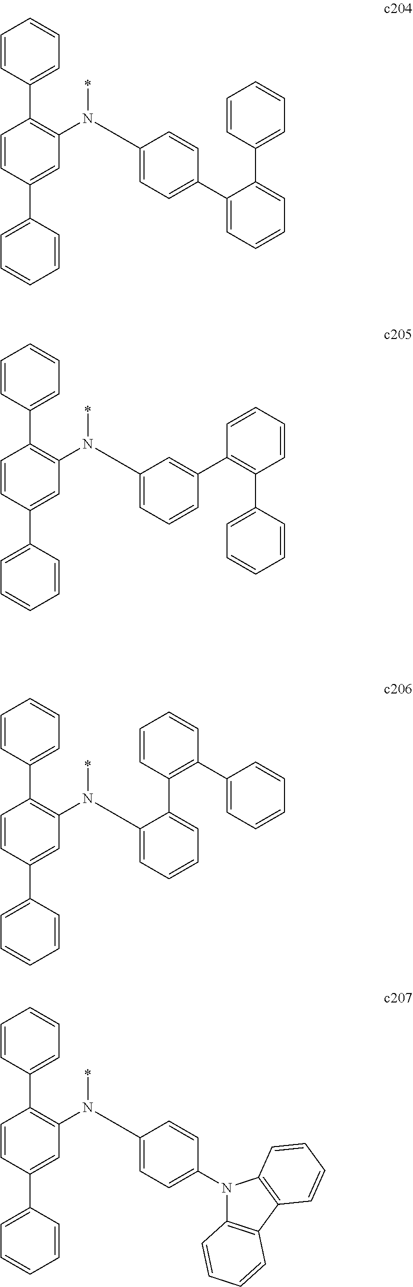

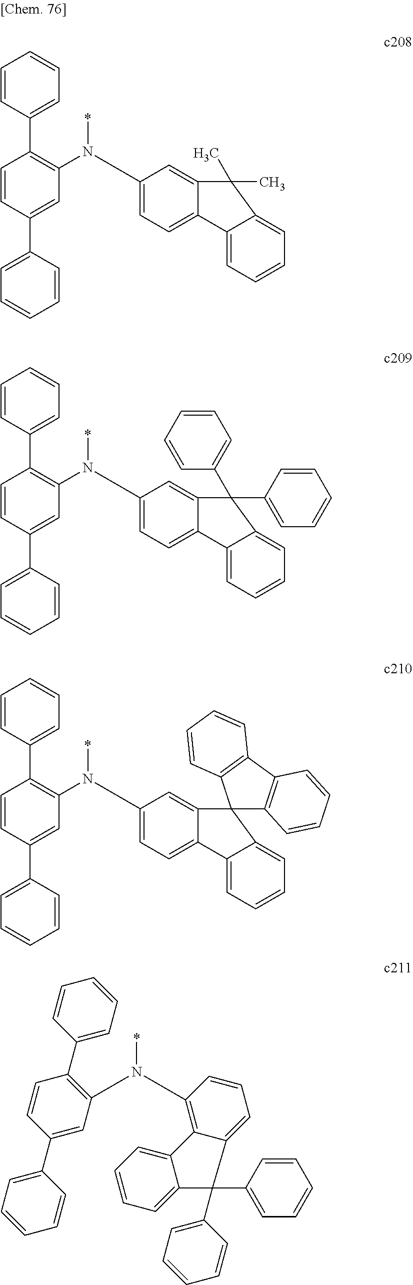

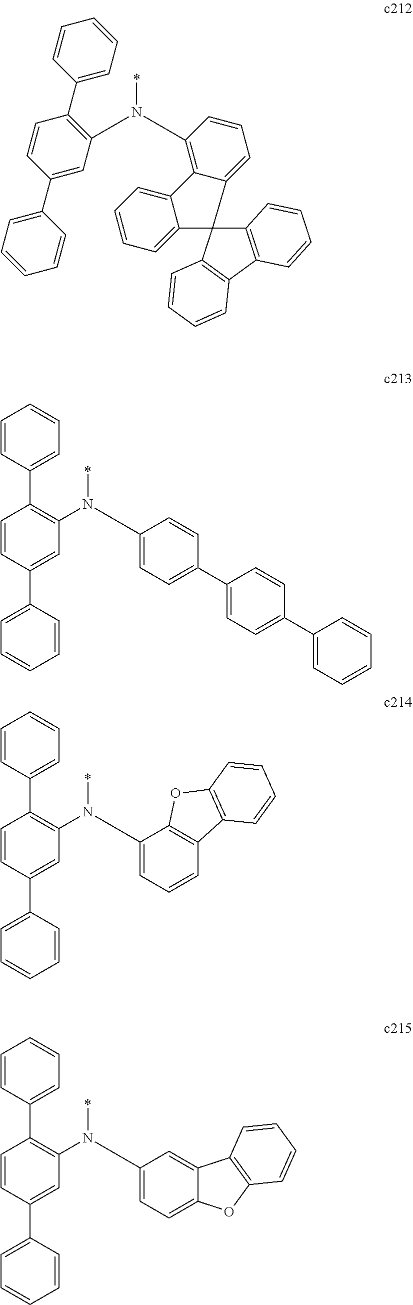

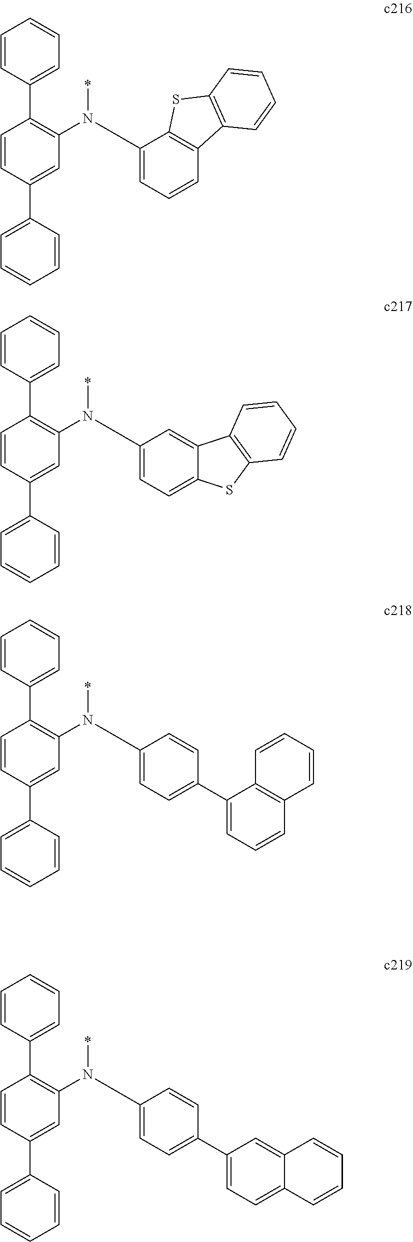

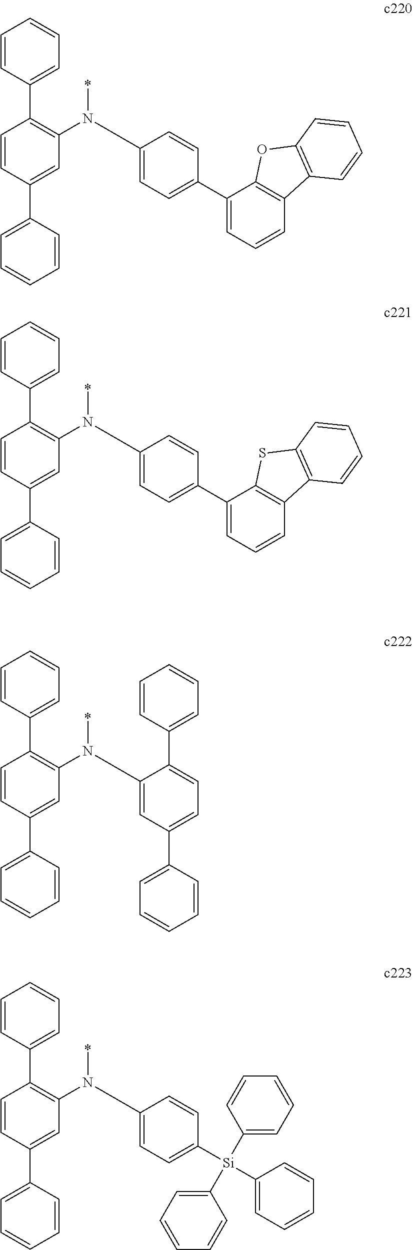

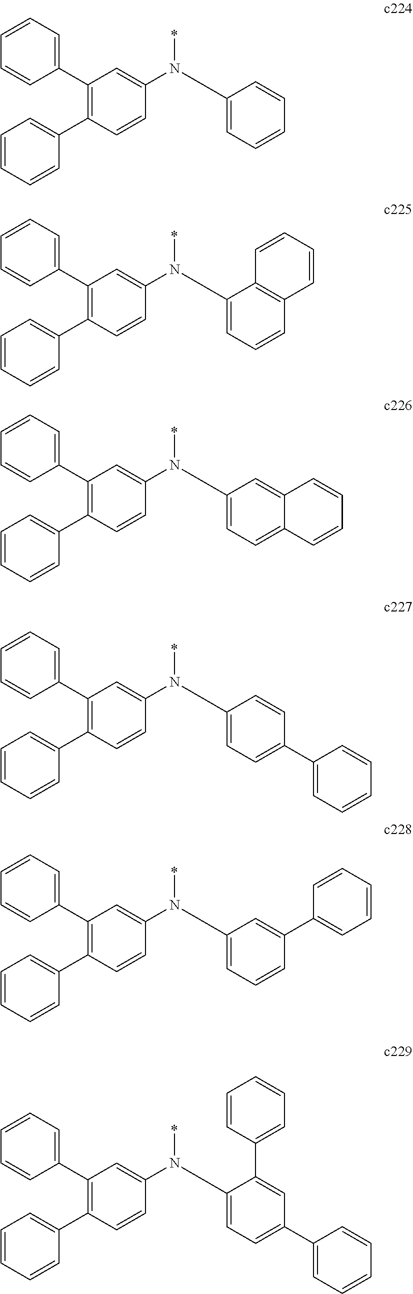

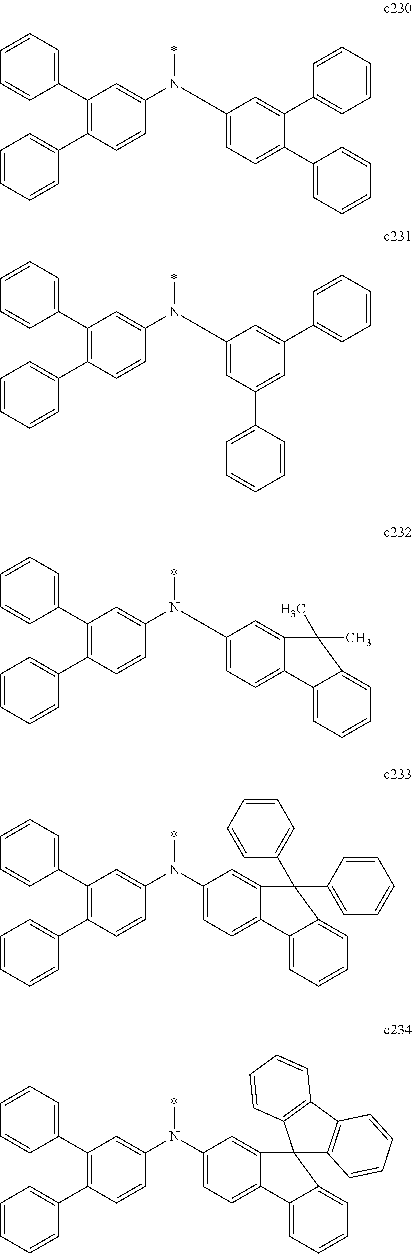 [SMILES: *N(c1ccc(-c2ccccc2)c(-c2ccccc2)c1)c1ccc(-c2ccccc2)c(-c2ccccc2)c1 ; *N(c1cc(-c2ccccc2)cc(-c2ccccc2)c1)c1ccc(-c2ccccc2)c(-c2ccccc2)c1 ; *N(c1ccc(-c2ccccc2)c(-c2ccccc2)c1)c1ccc2c(c1)C(C)(C)c1ccccc1-2 ; *N(c1ccc(-c2ccccc2)c(-c2ccccc2)c1)c1ccc2c(c1)C(c1ccccc1)(c1ccccc1)c1ccccc1-2 ; *N(c1ccc(-c2ccccc2)c(-c2ccccc2)c1)c1ccc2c(c1)C1(c3ccccc3-c3ccccc31)c1ccccc1-2]

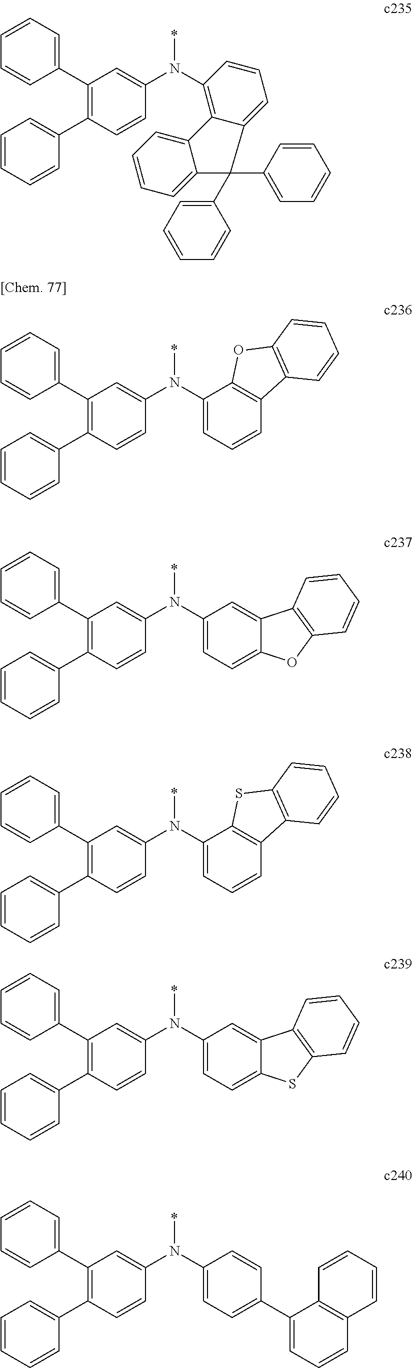

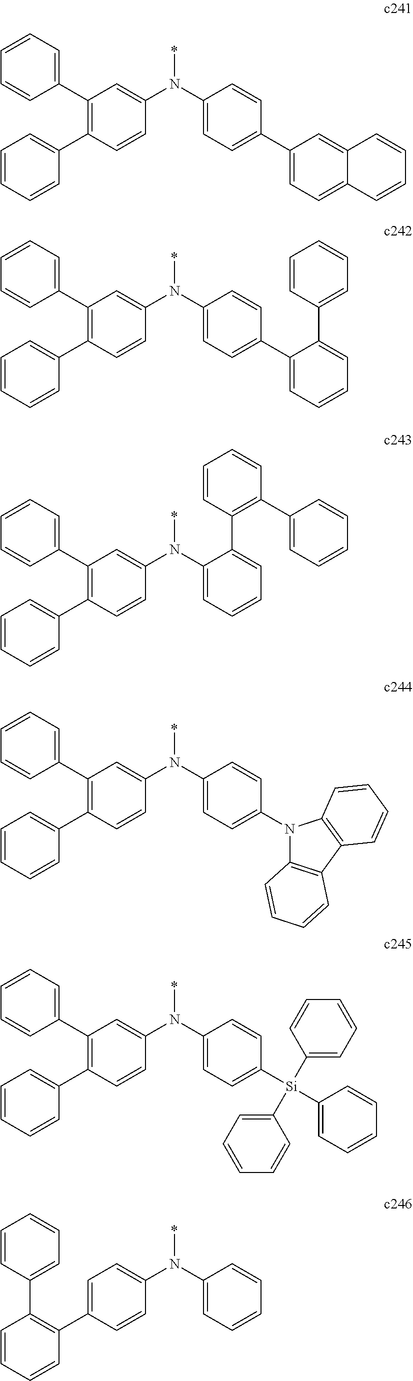

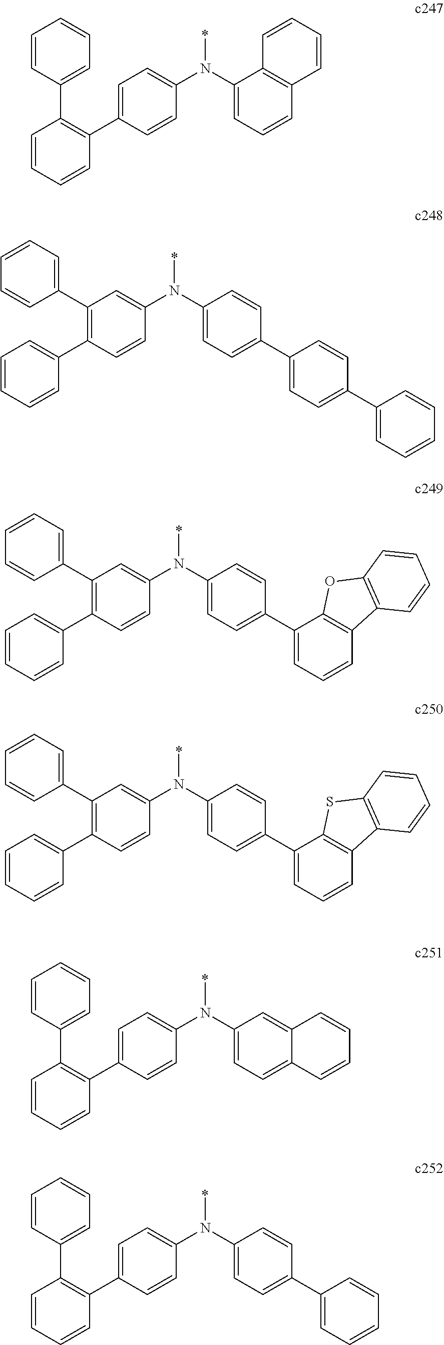 [SMILES: *N(c1ccc(-c2ccccc2-c2ccccc2)cc1)c1cccc2ccccc12 ; *N(c1ccc(-c2ccc(-c3ccccc3)cc2)cc1)c1ccc(-c2ccccc2)c(-c2ccccc2)c1 ; *N(c1ccc(-c2cccc3c2oc2ccccc23)cc1)c1ccc(-c2ccccc2)c(-c2ccccc2)c1 ; *N(c1ccc(-c2cccc3c2sc2ccccc23)cc1)c1ccc(-c2ccccc2)c(-c2ccccc2)c1 ; *N(c1ccc(-c2ccccc2-c2ccccc2)cc1)c1ccc2ccccc2c1 ; *N(c1ccc(-c2ccccc2)cc1)c1ccc(-c2ccccc2-c2ccccc2)cc1]

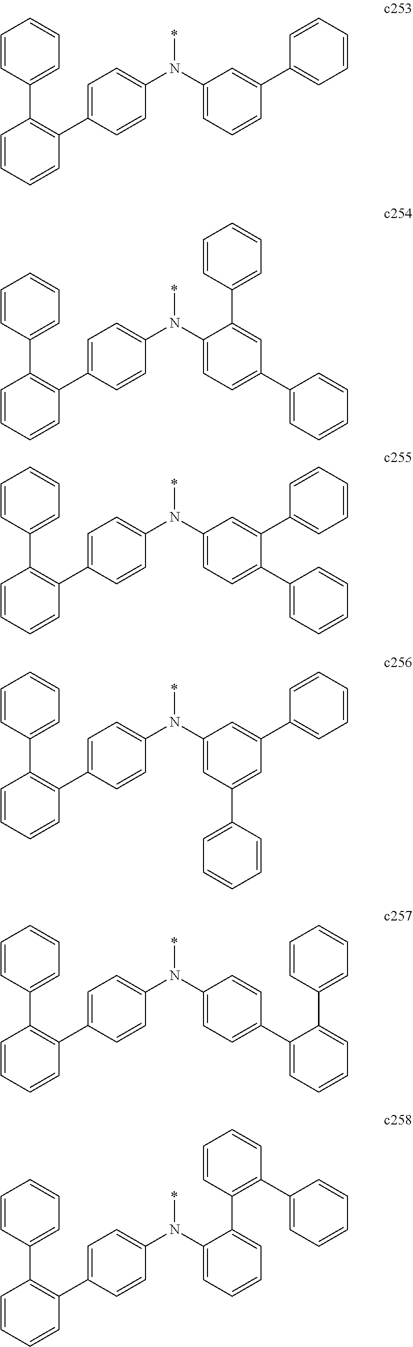

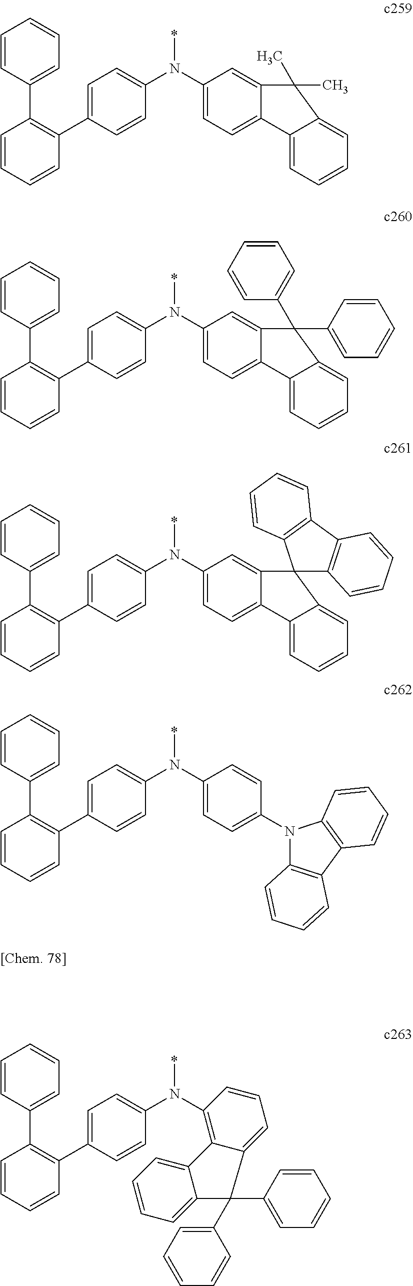

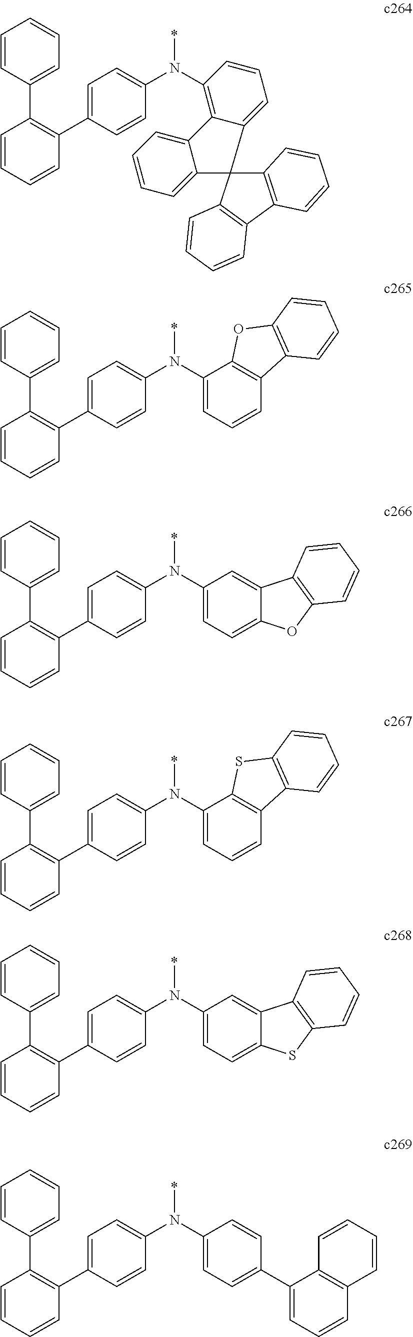

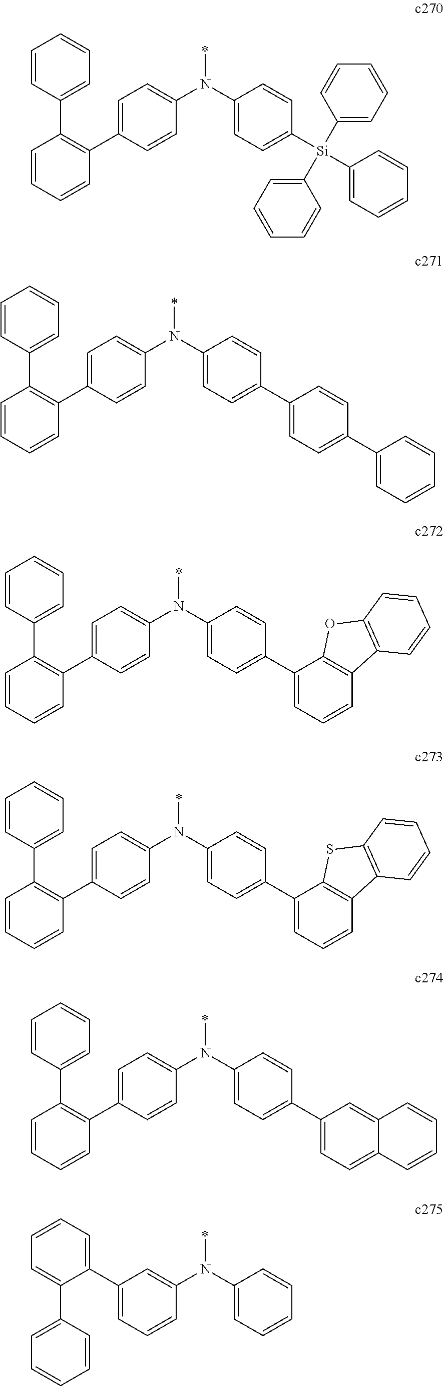 [SMILES: *N(c1ccc(-c2ccccc2-c2ccccc2)cc1)c1ccc([Si](c2ccccc2)(c2ccccc2)c2ccccc2)cc1 ; *N(c1ccc(-c2ccc(-c3ccccc3)cc2)cc1)c1ccc(-c2ccccc2-c2ccccc2)cc1 ; *N(c1ccc(-c2ccccc2-c2ccccc2)cc1)c1ccc(-c2cccc3c2oc2ccccc23)cc1 ; *N(c1ccc(-c2ccccc2-c2ccccc2)cc1)c1ccc(-c2cccc3c2sc2ccccc23)cc1 ; *N(c1ccc(-c2ccc3ccccc3c2)cc1)c1ccc(-c2ccccc2-c2ccccc2)cc1 ; *N(c1ccccc1)c1cccc(-c2ccccc2-c2ccccc2)c1]

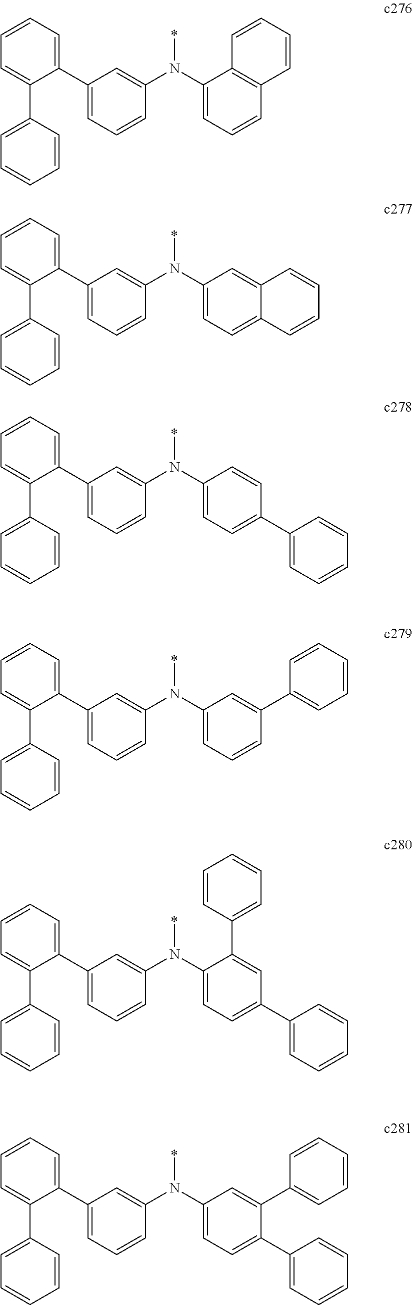

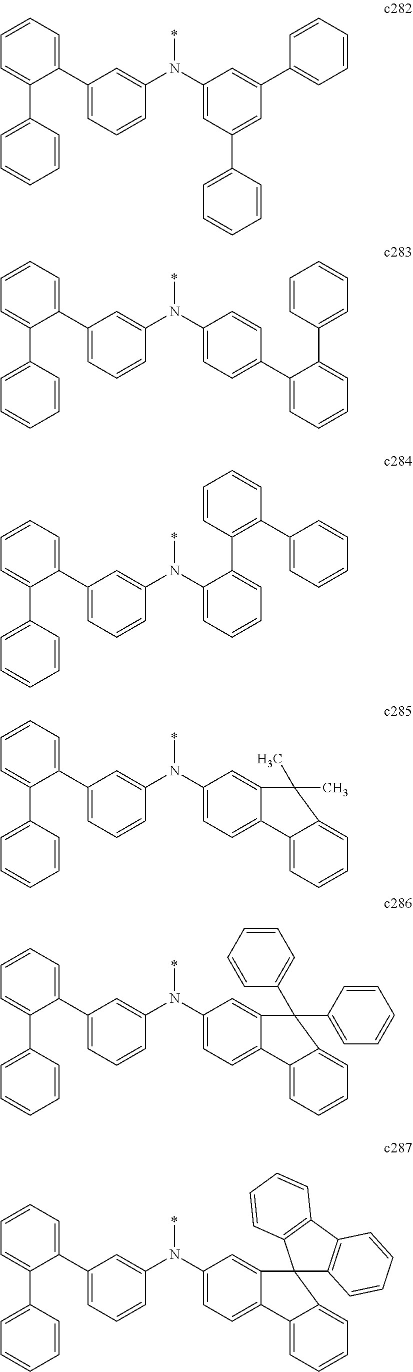

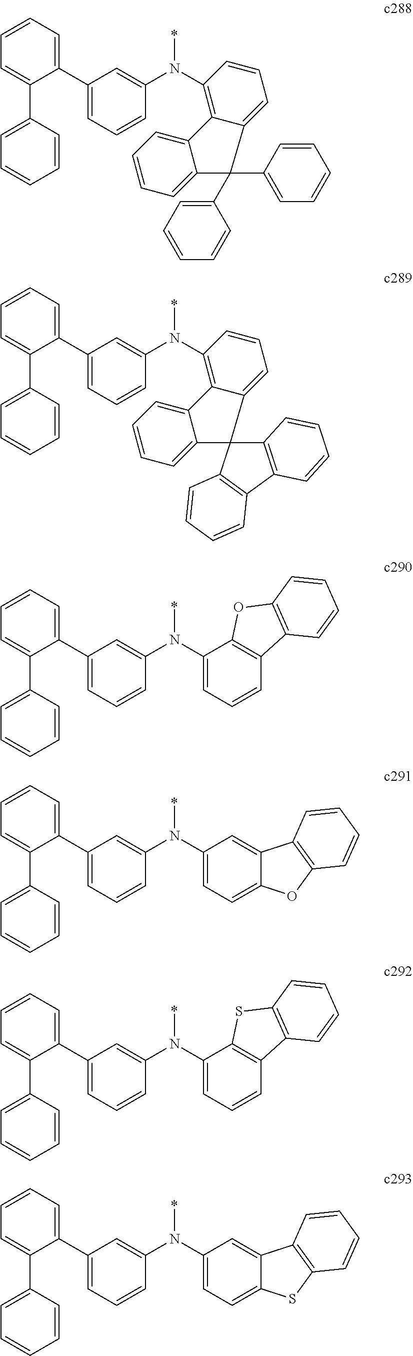

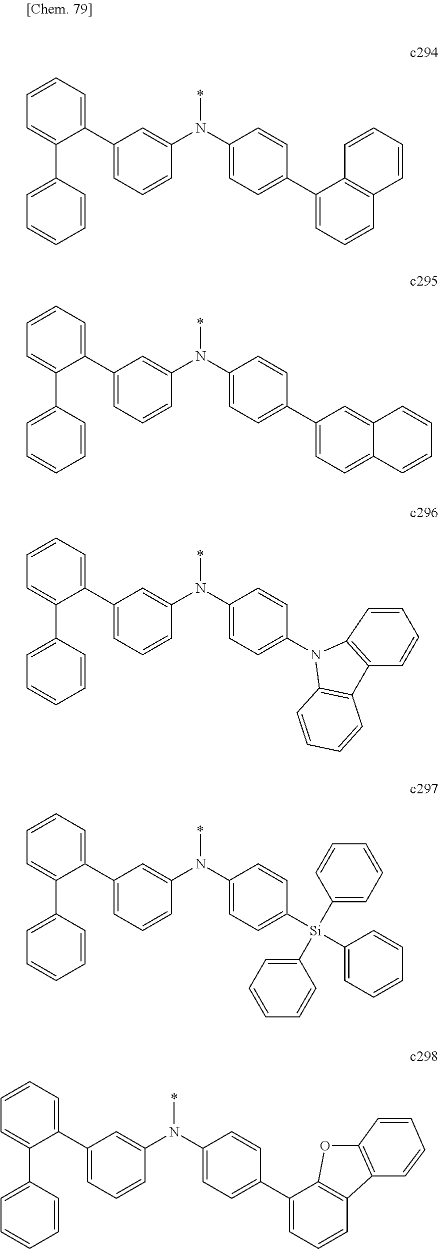 [SMILES: *N(c1ccc(-c2cccc3ccccc23)cc1)c1cccc(-c2ccccc2-c2ccccc2)c1 ; *N(c1ccc(-c2ccc3ccccc3c2)cc1)c1cccc(-c2ccccc2-c2ccccc2)c1 ; *N(c1ccc(-n2c3ccccc3c3ccccc32)cc1)c1cccc(-c2ccccc2-c2ccccc2)c1 ; *N(c1ccc([Si](c2ccccc2)(c2ccccc2)c2ccccc2)cc1)c1cccc(-c2ccccc2-c2ccccc2)c1 ; *N(c1ccc(-c2cccc3c2oc2ccccc23)cc1)c1cccc(-c2ccccc2-c2ccccc2)c1]

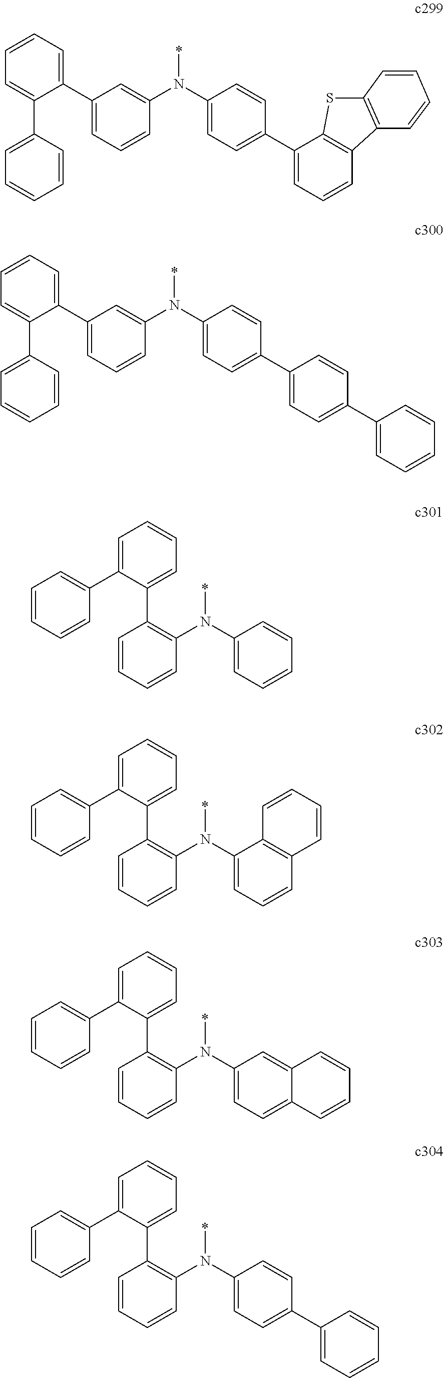

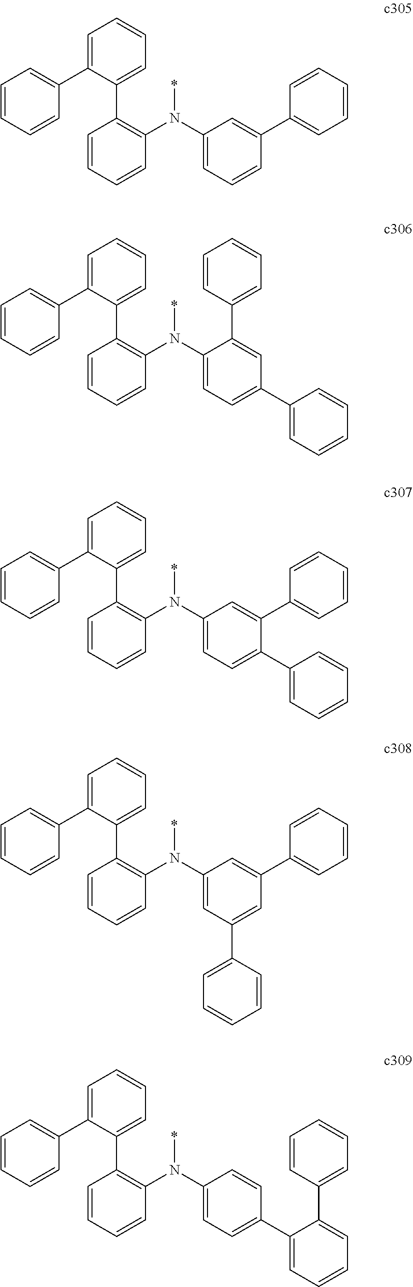 [SMILES: *N(c1cccc(-c2ccccc2)c1)c1ccccc1-c1ccccc1-c1ccccc1 ; *N(c1ccc(-c2ccccc2)cc1-c1ccccc1)c1ccccc1-c1ccccc1-c1ccccc1 ; *N(c1ccc(-c2ccccc2)c(-c2ccccc2)c1)c1ccccc1-c1ccccc1-c1ccccc1 ; *N(c1cc(-c2ccccc2)cc(-c2ccccc2)c1)c1ccccc1-c1ccccc1-c1ccccc1 ; *N(c1ccc(-c2ccccc2-c2ccccc2)cc1)c1ccccc1-c1ccccc1-c1ccccc1]

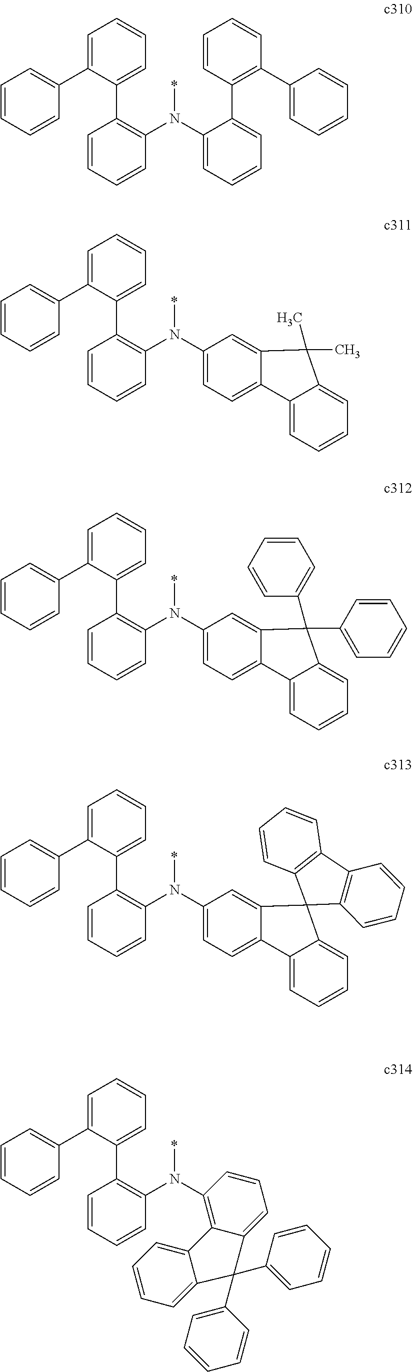 [SMILES: *N(c1ccccc1-c1ccccc1-c1ccccc1)c1ccccc1-c1ccccc1-c1ccccc1 ; *N(c1ccc2c(c1)C(C)(C)c1ccccc1-2)c1ccccc1-c1ccccc1-c1ccccc1 ; *N(c1ccc2c(c1)C(c1ccccc1)(c1ccccc1)c1ccccc1-2)c1ccccc1-c1ccccc1-c1ccccc1 ; *N(c1ccc2c(c1)C1(c3ccccc3-c3ccccc31)c1ccccc1-2)c1ccccc1-c1ccccc1-c1ccccc1 ; *N(c1ccccc1-c1ccccc1-c1ccccc1)c1cccc2c1-c1ccccc1C2(c1ccccc1)c1ccccc1]

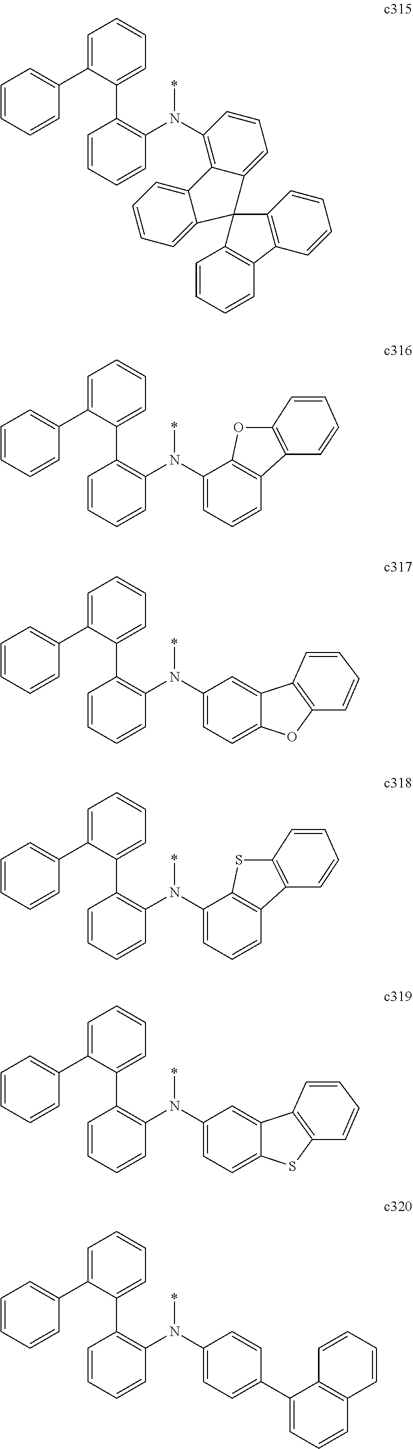

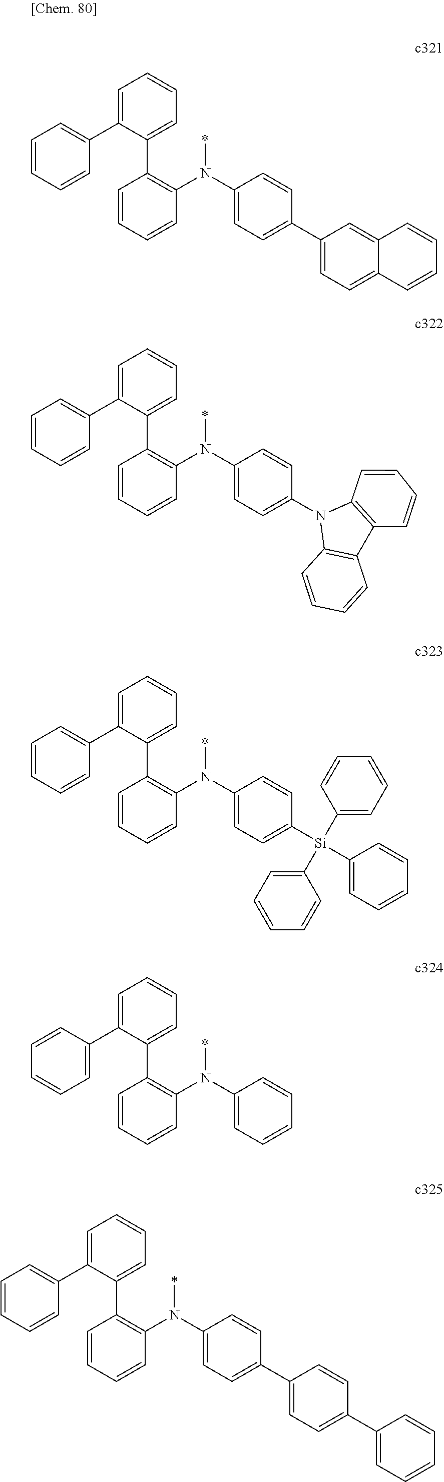 [SMILES: *N(c1ccc(-c2ccc3ccccc3c2)cc1)c1ccccc1-c1ccccc1-c1ccccc1 ; *N(c1ccc(-n2c3ccccc3c3ccccc32)cc1)c1ccccc1-c1ccccc1-c1ccccc1 ; *N(c1ccc([Si](c2ccccc2)(c2ccccc2)c2ccccc2)cc1)c1ccccc1-c1ccccc1-c1ccccc1 ; *N(c1ccccc1)c1ccccc1-c1ccccc1-c1ccccc1 ; *N(c1ccc(-c2ccc(-c3ccccc3)cc2)cc1)c1ccccc1-c1ccccc1-c1ccccc1]

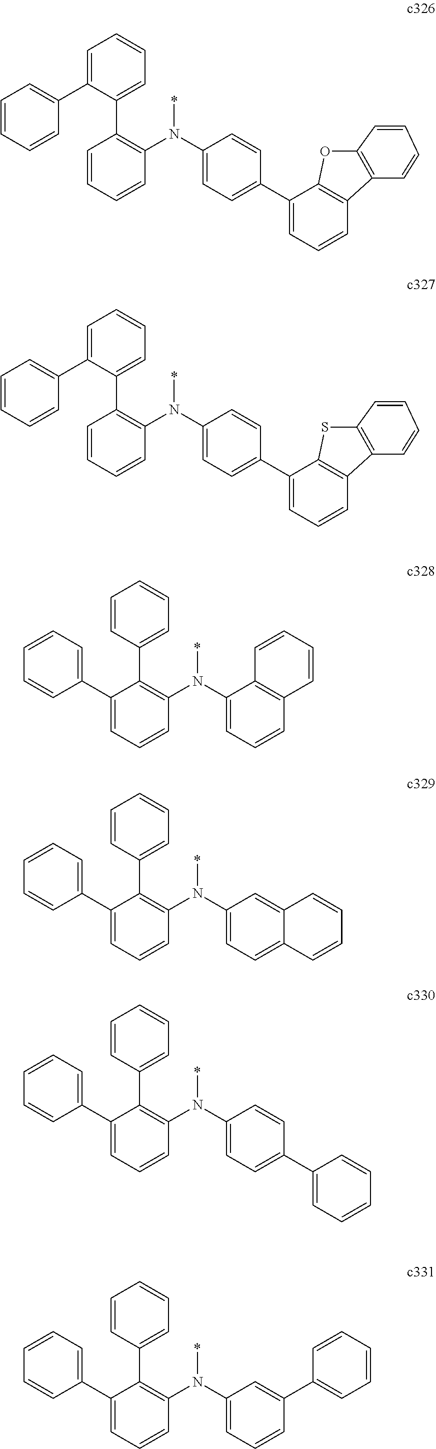

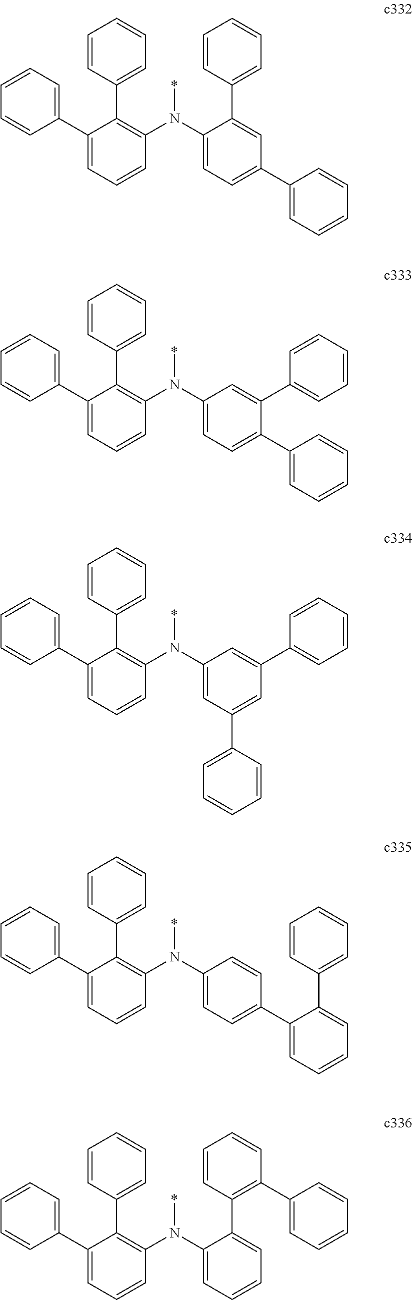 [SMILES: *N(c1ccc(-c2ccccc2)cc1-c1ccccc1)c1cccc(-c2ccccc2)c1-c1ccccc1 ; *N(c1ccc(-c2ccccc2)c(-c2ccccc2)c1)c1cccc(-c2ccccc2)c1-c1ccccc1 ; *N(c1cc(-c2ccccc2)cc(-c2ccccc2)c1)c1cccc(-c2ccccc2)c1-c1ccccc1 ; *N(c1ccc(-c2ccccc2-c2ccccc2)cc1)c1cccc(-c2ccccc2)c1-c1ccccc1 ; *N(c1ccccc1-c1ccccc1-c1ccccc1)c1cccc(-c2ccccc2)c1-c1ccccc1]

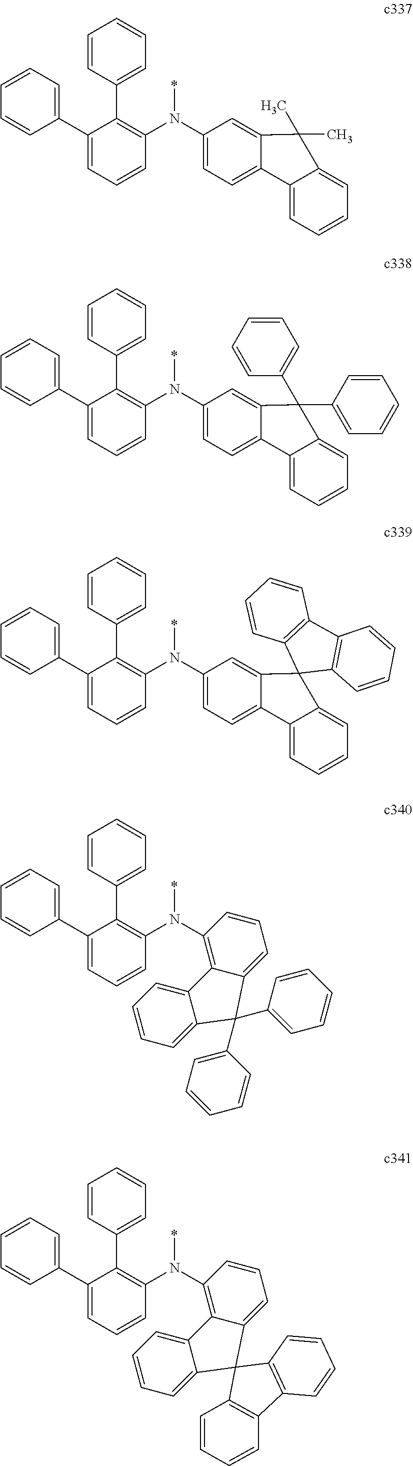 [SMILES: *N(c1ccc2c(c1)C(C)(C)c1ccccc1-2)c1cccc(-c2ccccc2)c1-c1ccccc1 ; *N(c1ccc2c(c1)C(c1ccccc1)(c1ccccc1)c1ccccc1-2)c1cccc(-c2ccccc2)c1-c1ccccc1 ; *N(c1ccc2c(c1)C1(c3ccccc3-c3ccccc31)c1ccccc1-2)c1cccc(-c2ccccc2)c1-c1ccccc1 ; *N(c1cccc(-c2ccccc2)c1-c1ccccc1)c1cccc2c1-c1ccccc1C2(c1ccccc1)c1ccccc1 ; *N(c1cccc(-c2ccccc2)c1-c1ccccc1)c1cccc2c1-c1ccccc1C21c2ccccc2-c2ccccc21]

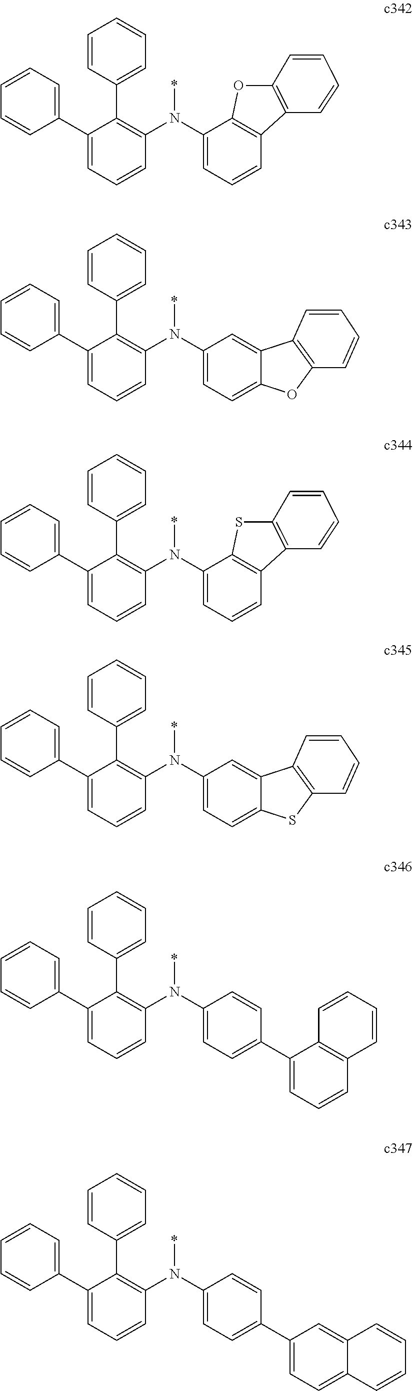

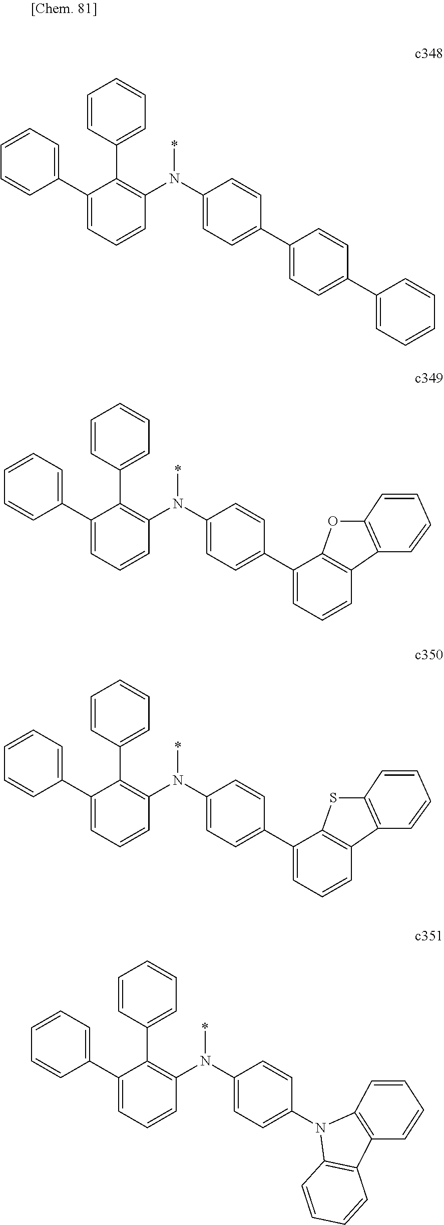 [SMILES: *N(c1ccc(-c2ccc(-c3ccccc3)cc2)cc1)c1cccc(-c2ccccc2)c1-c1ccccc1 ; *N(c1ccc(-c2cccc3c2oc2ccccc23)cc1)c1cccc(-c2ccccc2)c1-c1ccccc1 ; *N(c1ccc(-c2cccc3c2sc2ccccc23)cc1)c1cccc(-c2ccccc2)c1-c1ccccc1 ; *N(c1ccc(-n2c3ccccc3c3ccccc32)cc1)c1cccc(-c2ccccc2)c1-c1ccccc1]

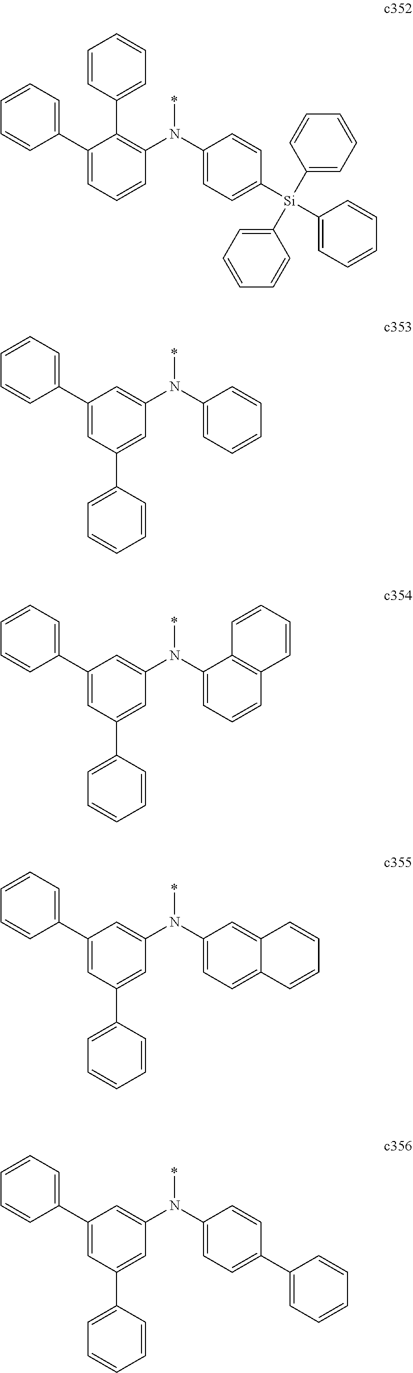 [SMILES: *N(c1ccc([Si](c2ccccc2)(c2ccccc2)c2ccccc2)cc1)c1cccc(-c2ccccc2)c1-c1ccccc1 ; *N(c1ccccc1)c1cc(-c2ccccc2)cc(-c2ccccc2)c1 ; *N(c1cc(-c2ccccc2)cc(-c2ccccc2)c1)c1cccc2ccccc12 ; *N(c1cc(-c2ccccc2)cc(-c2ccccc2)c1)c1ccc2ccccc2c1 ; *N(c1ccc(-c2ccccc2)cc1)c1cc(-c2ccccc2)cc(-c2ccccc2)c1]

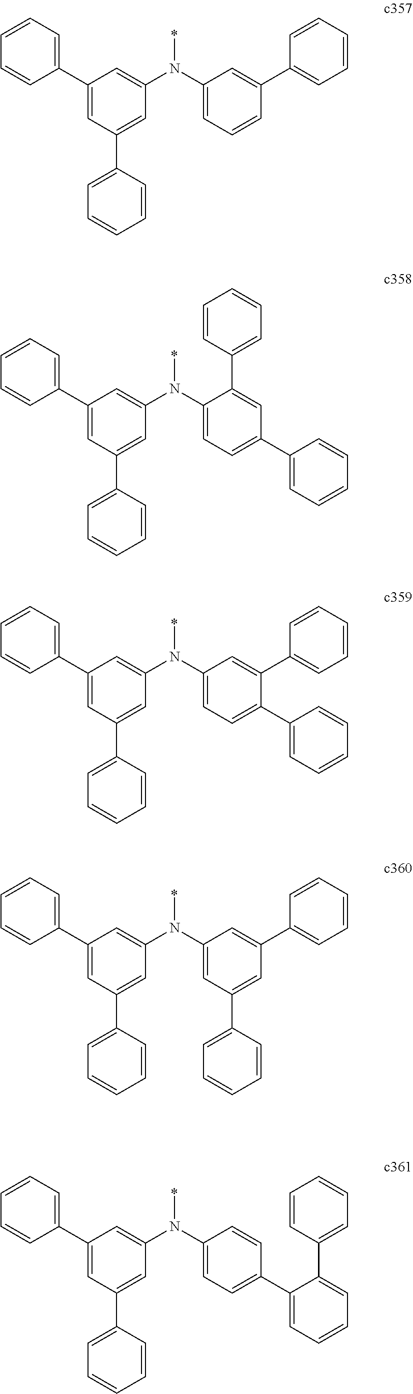 [SMILES: *N(c1cccc(-c2ccccc2)c1)c1cc(-c2ccccc2)cc(-c2ccccc2)c1 ; *N(c1cc(-c2ccccc2)cc(-c2ccccc2)c1)c1ccc(-c2ccccc2)cc1-c1ccccc1 ; *N(c1cc(-c2ccccc2)cc(-c2ccccc2)c1)c1ccc(-c2ccccc2)c(-c2ccccc2)c1 ; *N(c1cc(-c2ccccc2)cc(-c2ccccc2)c1)c1cc(-c2ccccc2)cc(-c2ccccc2)c1 ; *N(c1ccc(-c2ccccc2-c2ccccc2)cc1)c1cc(-c2ccccc2)cc(-c2ccccc2)c1]

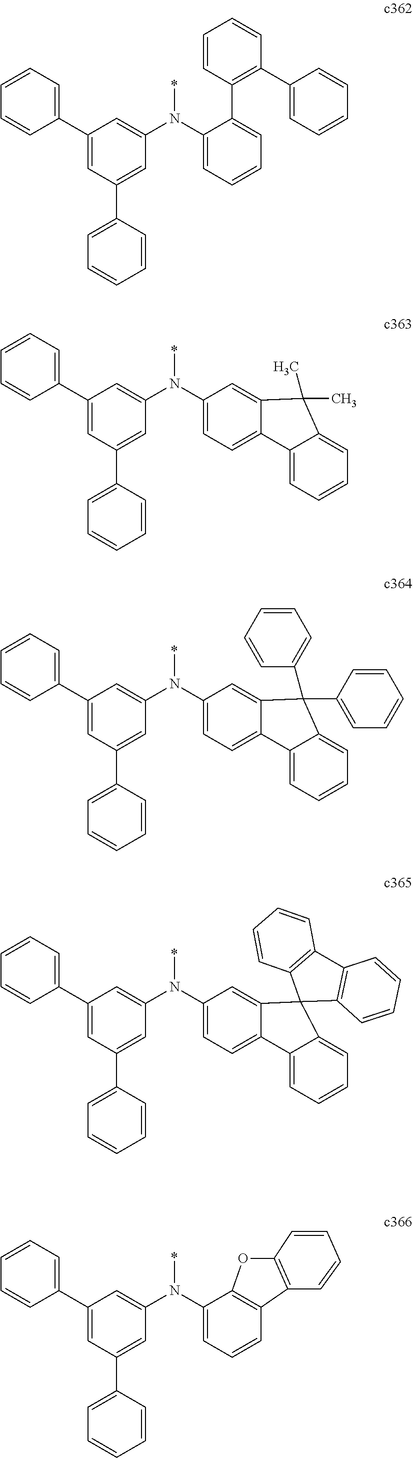 [SMILES: *N(c1cc(-c2ccccc2)cc(-c2ccccc2)c1)c1ccccc1-c1ccccc1-c1ccccc1 ; *N(c1cc(-c2ccccc2)cc(-c2ccccc2)c1)c1ccc2c(c1)C(C)(C)c1ccccc1-2 ; *N(c1cc(-c2ccccc2)cc(-c2ccccc2)c1)c1ccc2c(c1)C(c1ccccc1)(c1ccccc1)c1ccccc1-2 ; *N(c1cc(-c2ccccc2)cc(-c2ccccc2)c1)c1ccc2c(c1)C1(c3ccccc3-c3ccccc31)c1ccccc1-2 ; *N(c1cc(-c2ccccc2)cc(-c2ccccc2)c1)c1cccc2c1oc1ccccc12]

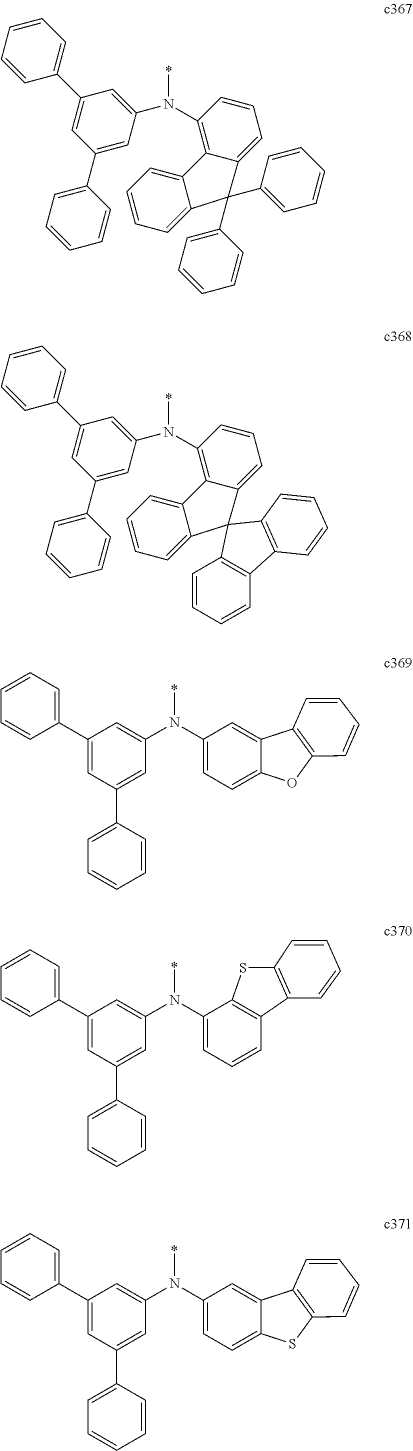 [SMILES: *N(c1cc(-c2ccccc2)cc(-c2ccccc2)c1)c1cccc2c1-c1ccccc1C2(c1ccccc1)c1ccccc1 ; *N(c1cc(-c2ccccc2)cc(-c2ccccc2)c1)c1cccc2c1-c1ccccc1C21c2ccccc2-c2ccccc21 ; *N(c1cc(-c2ccccc2)cc(-c2ccccc2)c1)c1ccc2oc3ccccc3c2c1 ; *N(c1cc(-c2ccccc2)cc(-c2ccccc2)c1)c1cccc2c1sc1ccccc12 ; *N(c1cc(-c2ccccc2)cc(-c2ccccc2)c1)c1ccc2sc3ccccc3c2c1]

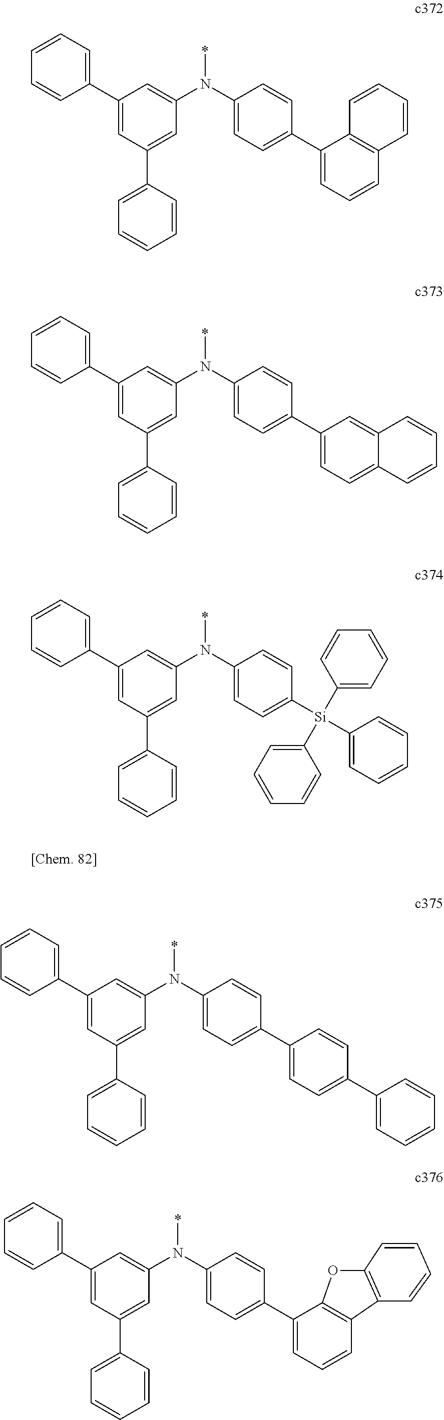 [SMILES: *N(c1ccc(-c2cccc3ccccc23)cc1)c1cc(-c2ccccc2)cc(-c2ccccc2)c1 ; *N(c1ccc(-c2ccc3ccccc3c2)cc1)c1cc(-c2ccccc2)cc(-c2ccccc2)c1 ; *N(c1ccc([Si](c2ccccc2)(c2ccccc2)c2ccccc2)cc1)c1cc(-c2ccccc2)cc(-c2ccccc2)c1 ; *N(c1ccc(-c2ccc(-c3ccccc3)cc2)cc1)c1cc(-c2ccccc2)cc(-c2ccccc2)c1 ; *N(c1ccc(-c2cccc3c2oc2ccccc23)cc1)c1cc(-c2ccccc2)cc(-c2ccccc2)c1]

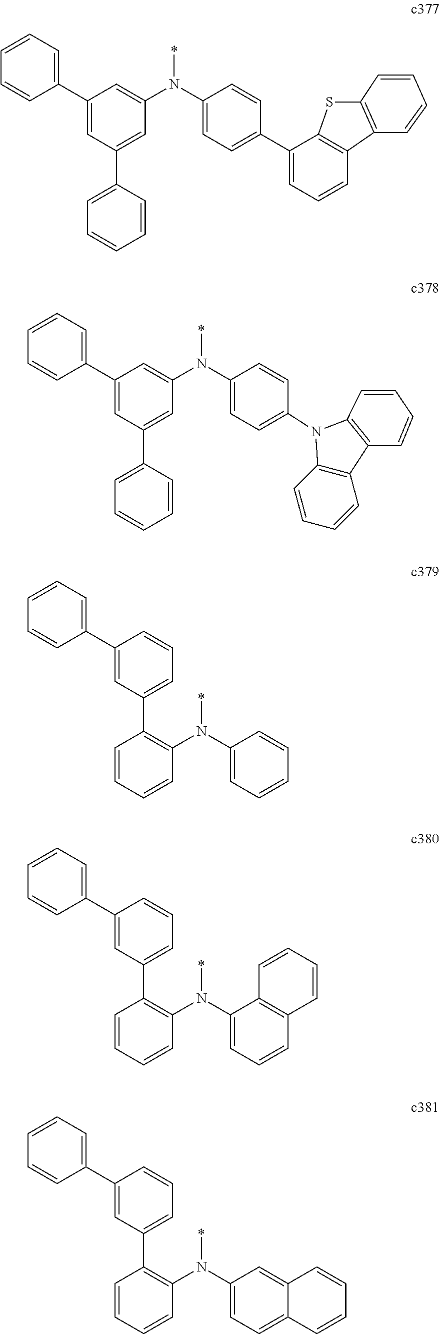 [SMILES: *N(c1ccc(-c2cccc3c2sc2ccccc23)cc1)c1cc(-c2ccccc2)cc(-c2ccccc2)c1 ; *N(c1ccc(-n2c3ccccc3c3ccccc32)cc1)c1cc(-c2ccccc2)cc(-c2ccccc2)c1 ; *N(c1ccccc1)c1ccccc1-c1cccc(-c2ccccc2)c1 ; *N(c1ccccc1-c1cccc(-c2ccccc2)c1)c1cccc2ccccc12 ; *N(c1ccc2ccccc2c1)c1ccccc1-c1cccc(-c2ccccc2)c1]

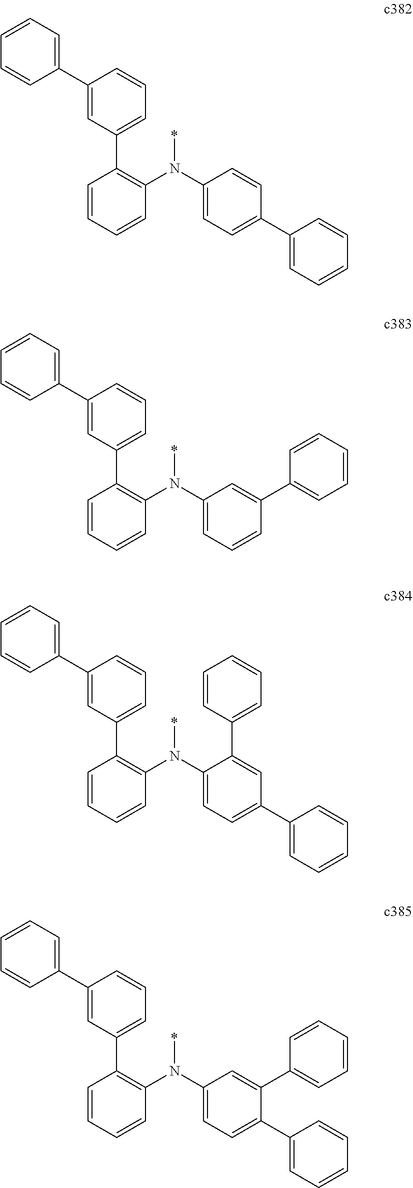 [SMILES: *N(c1ccc(-c2ccccc2)cc1)c1ccccc1-c1cccc(-c2ccccc2)c1 ; *N(c1cccc(-c2ccccc2)c1)c1ccccc1-c1cccc(-c2ccccc2)c1 ; *N(c1ccccc1-c1cccc(-c2ccccc2)c1)c1ccc(-c2ccccc2)cc1-c1ccccc1 ; *N(c1ccc(-c2ccccc2)c(-c2ccccc2)c1)c1ccccc1-c1cccc(-c2ccccc2)c1]

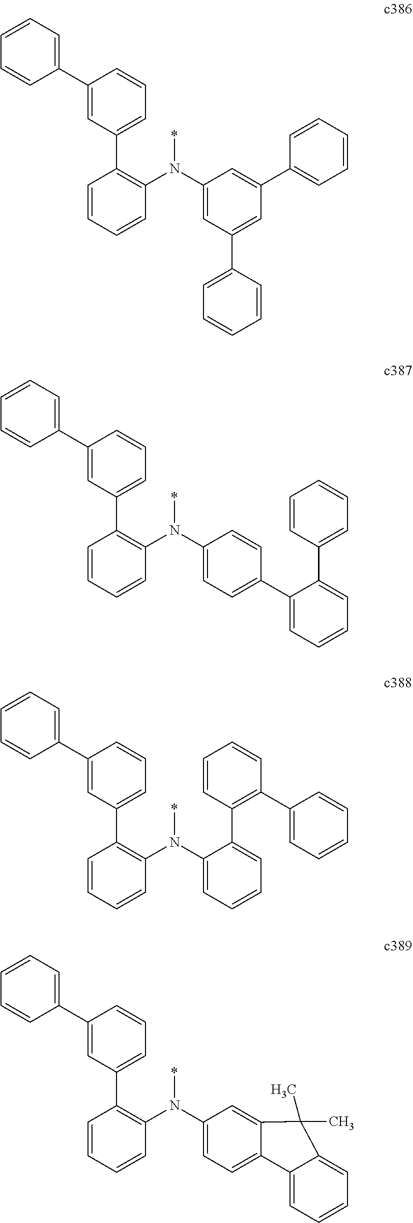 [SMILES: *N(c1cc(-c2ccccc2)cc(-c2ccccc2)c1)c1ccccc1-c1cccc(-c2ccccc2)c1 ; *N(c1ccc(-c2ccccc2-c2ccccc2)cc1)c1ccccc1-c1cccc(-c2ccccc2)c1 ; *N(c1ccccc1-c1cccc(-c2ccccc2)c1)c1ccccc1-c1ccccc1-c1ccccc1 ; *N(c1ccc2c(c1)C(C)(C)c1ccccc1-2)c1ccccc1-c1cccc(-c2ccccc2)c1]

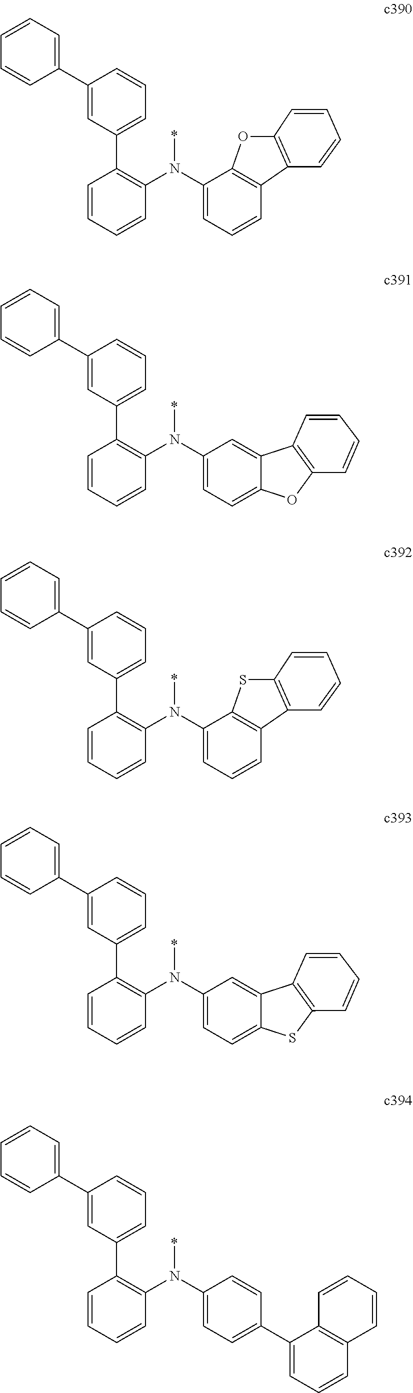 [SMILES: *N(c1ccccc1-c1cccc(-c2ccccc2)c1)c1cccc2c1oc1ccccc12 ; *N(c1ccc2oc3ccccc3c2c1)c1ccccc1-c1cccc(-c2ccccc2)c1 ; *N(c1ccccc1-c1cccc(-c2ccccc2)c1)c1cccc2c1sc1ccccc12 ; *N(c1ccc2sc3ccccc3c2c1)c1ccccc1-c1cccc(-c2ccccc2)c1 ; *N(c1ccc(-c2cccc3ccccc23)cc1)c1ccccc1-c1cccc(-c2ccccc2)c1]

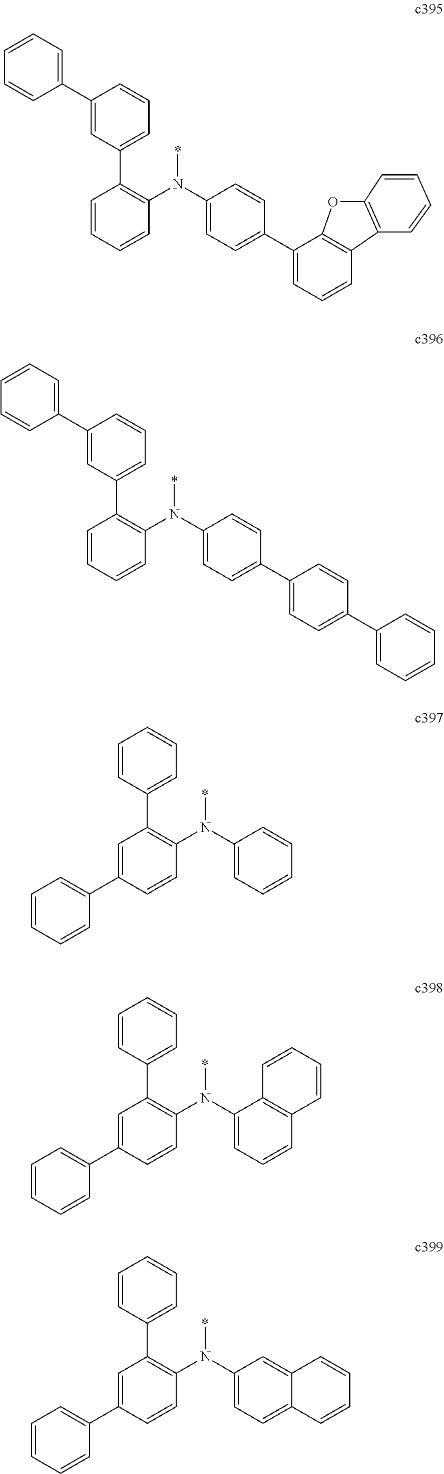 [SMILES: *N(c1ccc(-c2cccc3c2oc2ccccc23)cc1)c1ccccc1-c1cccc(-c2ccccc2)c1 ; *N(c1ccc(-c2ccc(-c3ccccc3)cc2)cc1)c1ccccc1-c1cccc(-c2ccccc2)c1 ; *N(c1ccccc1)c1ccc(-c2ccccc2)cc1-c1ccccc1 ; *N(c1ccc(-c2ccccc2)cc1-c1ccccc1)c1cccc2ccccc12 ; *N(c1ccc2ccccc2c1)c1ccc(-c2ccccc2)cc1-c1ccccc1]

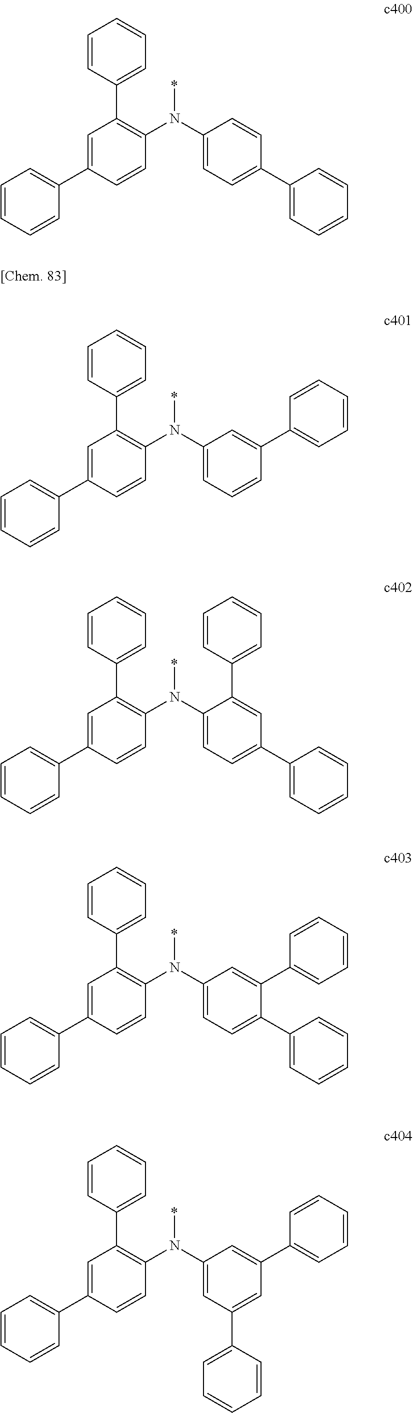 [SMILES: *N(c1ccc(-c2ccccc2)cc1)c1ccc(-c2ccccc2)cc1-c1ccccc1 ; *N(c1cccc(-c2ccccc2)c1)c1ccc(-c2ccccc2)cc1-c1ccccc1 ; *N(c1ccc(-c2ccccc2)cc1-c1ccccc1)c1ccc(-c2ccccc2)cc1-c1ccccc1 ; *N(c1ccc(-c2ccccc2)c(-c2ccccc2)c1)c1ccc(-c2ccccc2)cc1-c1ccccc1 ; *N(c1cc(-c2ccccc2)cc(-c2ccccc2)c1)c1ccc(-c2ccccc2)cc1-c1ccccc1]

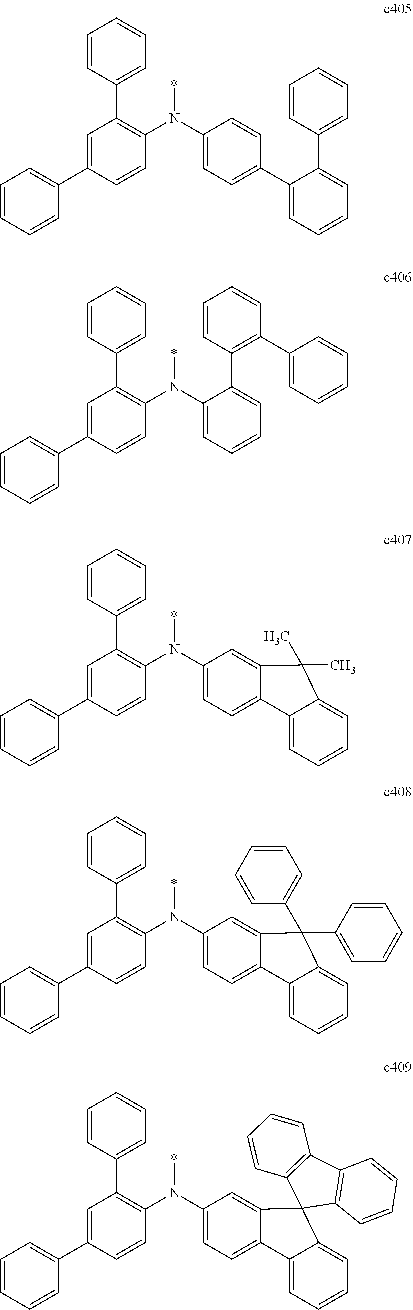 [SMILES: *N(c1ccc(-c2ccccc2-c2ccccc2)cc1)c1ccc(-c2ccccc2)cc1-c1ccccc1 ; *N(c1ccc(-c2ccccc2)cc1-c1ccccc1)c1ccccc1-c1ccccc1-c1ccccc1 ; *N(c1ccc2c(c1)C(C)(C)c1ccccc1-2)c1ccc(-c2ccccc2)cc1-c1ccccc1 ; *N(c1ccc2c(c1)C(c1ccccc1)(c1ccccc1)c1ccccc1-2)c1ccc(-c2ccccc2)cc1-c1ccccc1 ; *N(c1ccc2c(c1)C1(c3ccccc3-c3ccccc31)c1ccccc1-2)c1ccc(-c2ccccc2)cc1-c1ccccc1]

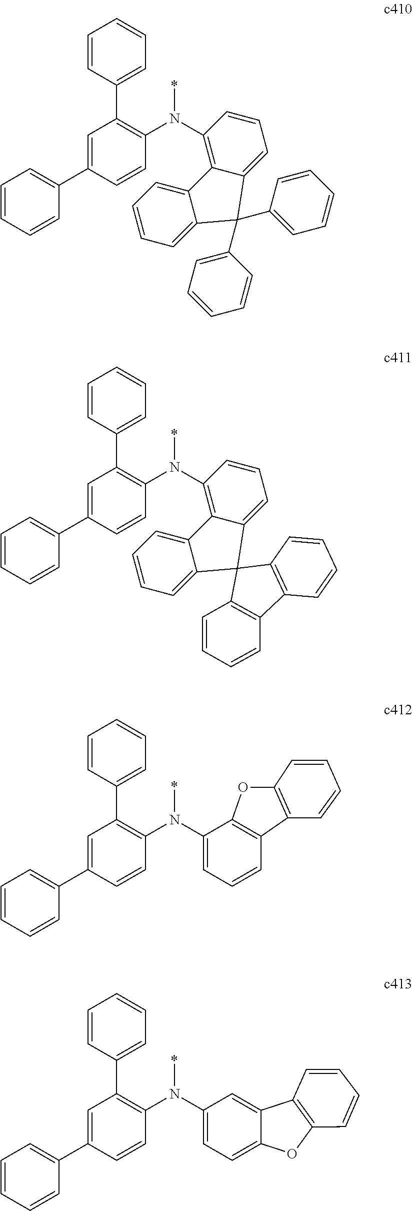 [SMILES: *N(c1ccc(-c2ccccc2)cc1-c1ccccc1)c1cccc2c1-c1ccccc1C2(c1ccccc1)c1ccccc1 ; *N(c1ccc(-c2ccccc2)cc1-c1ccccc1)c1cccc2c1-c1ccccc1C21c2ccccc2-c2ccccc21 ; *N(c1ccc(-c2ccccc2)cc1-c1ccccc1)c1cccc2c1oc1ccccc12 ; *N(c1ccc2oc3ccccc3c2c1)c1ccc(-c2ccccc2)cc1-c1ccccc1]

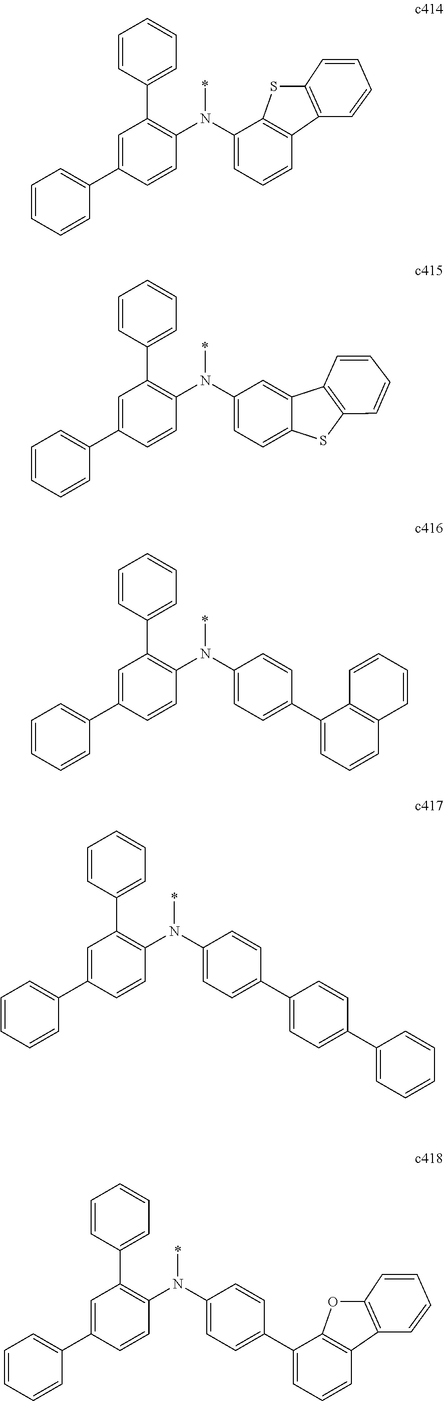 [SMILES: *N(c1ccc(-c2ccccc2)cc1-c1ccccc1)c1cccc2c1sc1ccccc12 ; *N(c1ccc2sc3ccccc3c2c1)c1ccc(-c2ccccc2)cc1-c1ccccc1 ; *N(c1ccc(-c2cccc3ccccc23)cc1)c1ccc(-c2ccccc2)cc1-c1ccccc1 ; *N(c1ccc(-c2ccc(-c3ccccc3)cc2)cc1)c1ccc(-c2ccccc2)cc1-c1ccccc1 ; *N(c1ccc(-c2cccc3c2oc2ccccc23)cc1)c1ccc(-c2ccccc2)cc1-c1ccccc1]

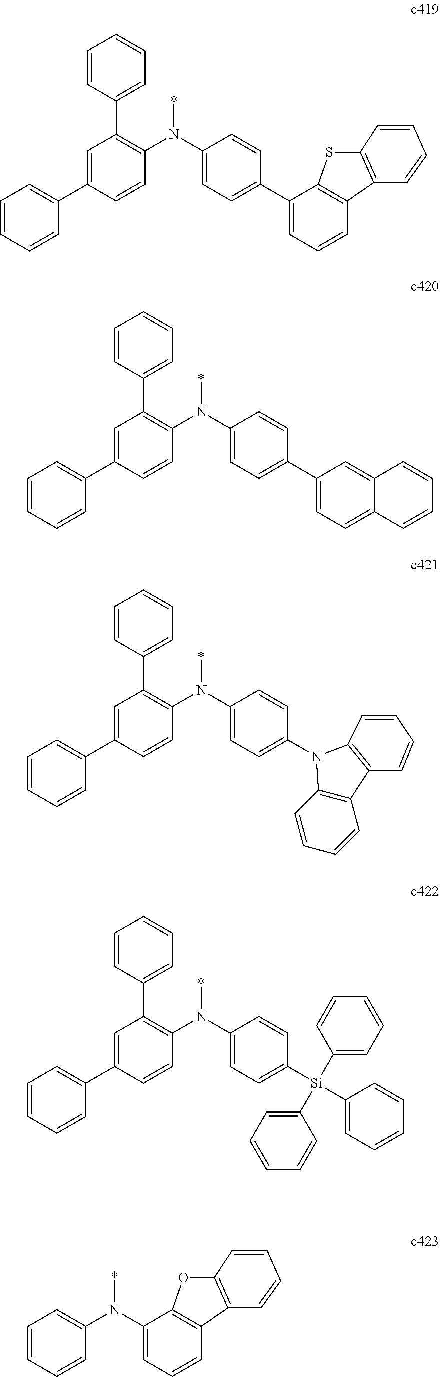 [SMILES: *N(c1ccc(-c2cccc3c2sc2ccccc23)cc1)c1ccc(-c2ccccc2)cc1-c1ccccc1 ; *N(c1ccc(-c2ccc3ccccc3c2)cc1)c1ccc(-c2ccccc2)cc1-c1ccccc1 ; *N(c1ccc(-n2c3ccccc3c3ccccc32)cc1)c1ccc(-c2ccccc2)cc1-c1ccccc1 ; *N(c1ccc([Si](c2ccccc2)(c2ccccc2)c2ccccc2)cc1)c1ccc(-c2ccccc2)cc1-c1ccccc1 ; *N(c1ccccc1)c1cccc2c1oc1ccccc12]

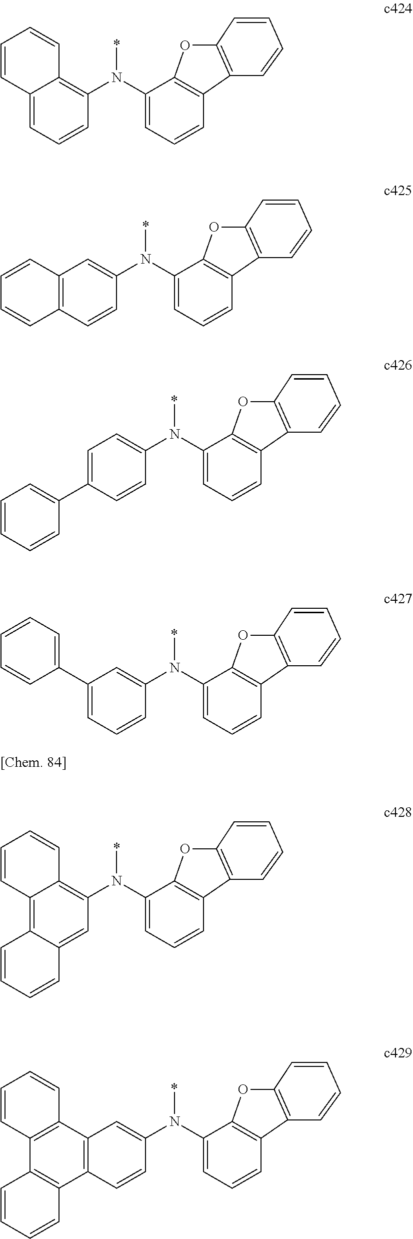 [SMILES: *N(c1cccc2ccccc12)c1cccc2c1oc1ccccc12 ; *N(c1ccc2ccccc2c1)c1cccc2c1oc1ccccc12 ; *N(c1ccc(-c2ccccc2)cc1)c1cccc2c1oc1ccccc12 ; *N(c1cccc(-c2ccccc2)c1)c1cccc2c1oc1ccccc12 ; *N(c1cc2ccccc2c2ccccc12)c1cccc2c1oc1ccccc12 ; *N(c1ccc2c3ccccc3c3ccccc3c2c1)c1cccc2c1oc1ccccc12]

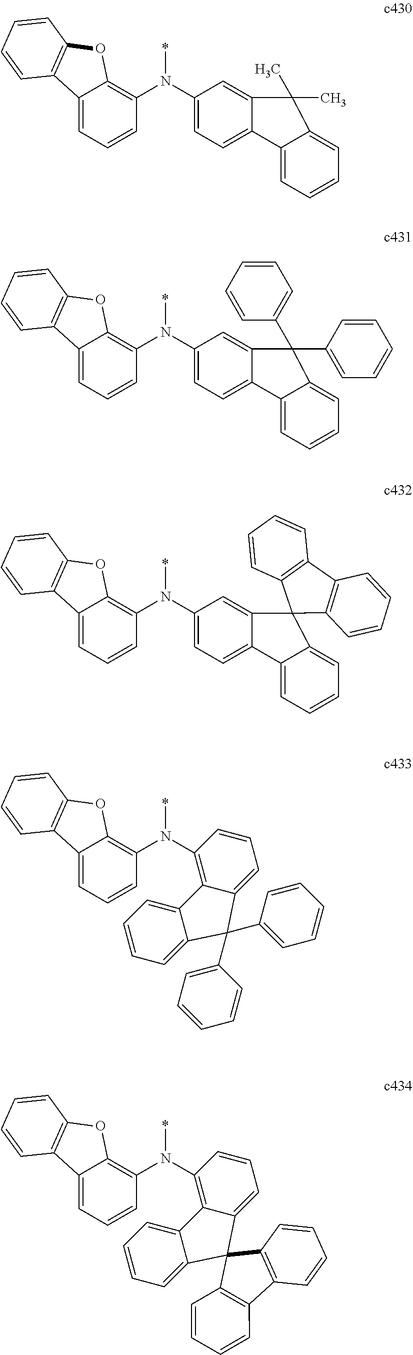

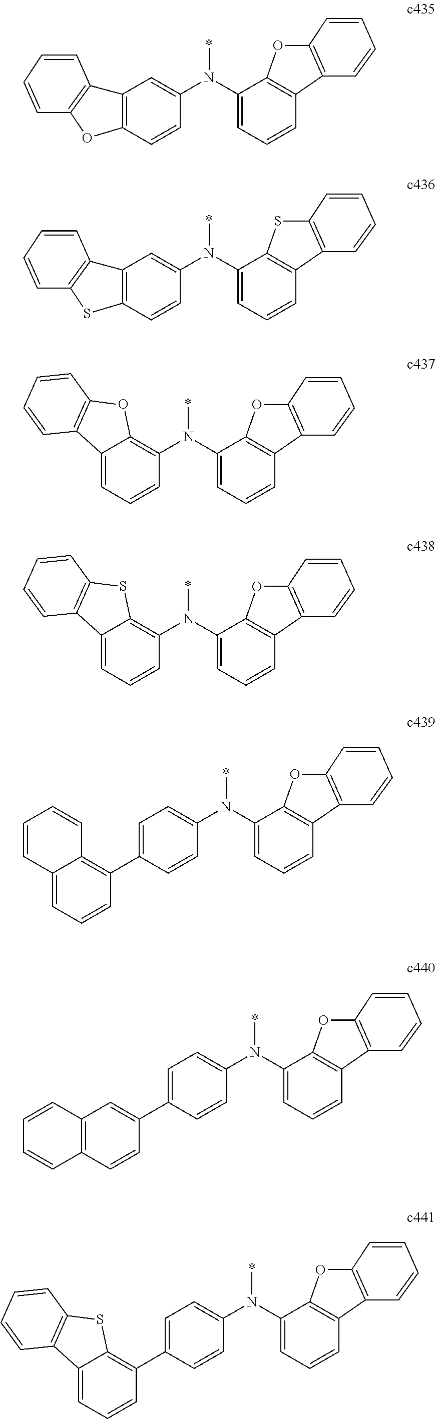

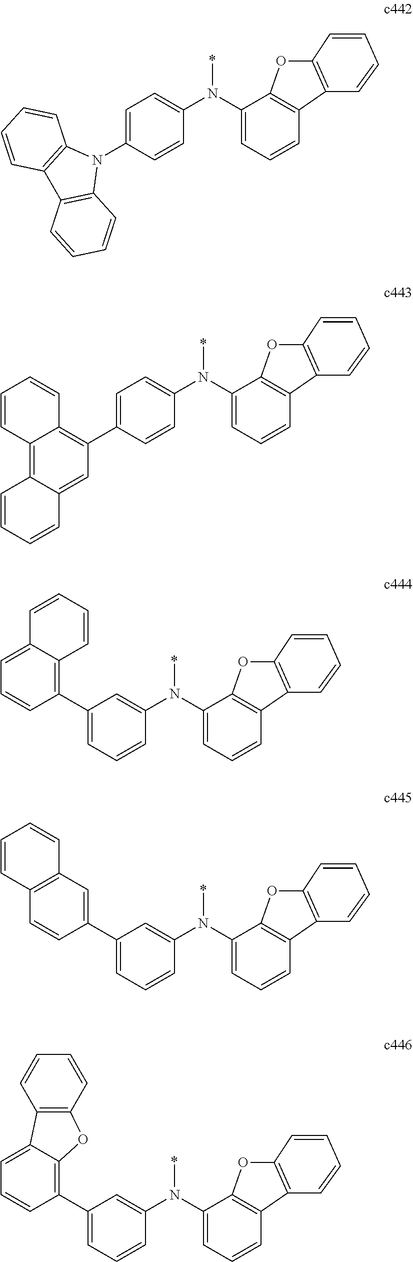 [SMILES: *N(c1ccc(-n2c3ccccc3c3ccccc32)cc1)c1cccc2c1oc1ccccc12 ; *N(c1ccc(-c2cc3ccccc3c3ccccc23)cc1)c1cccc2c1oc1ccccc12 ; *N(c1cccc(-c2cccc3ccccc23)c1)c1cccc2c1oc1ccccc12 ; *N(c1cccc(-c2ccc3ccccc3c2)c1)c1cccc2c1oc1ccccc12 ; *N(c1cccc(-c2cccc3c2oc2ccccc23)c1)c1cccc2c1oc1ccccc12]

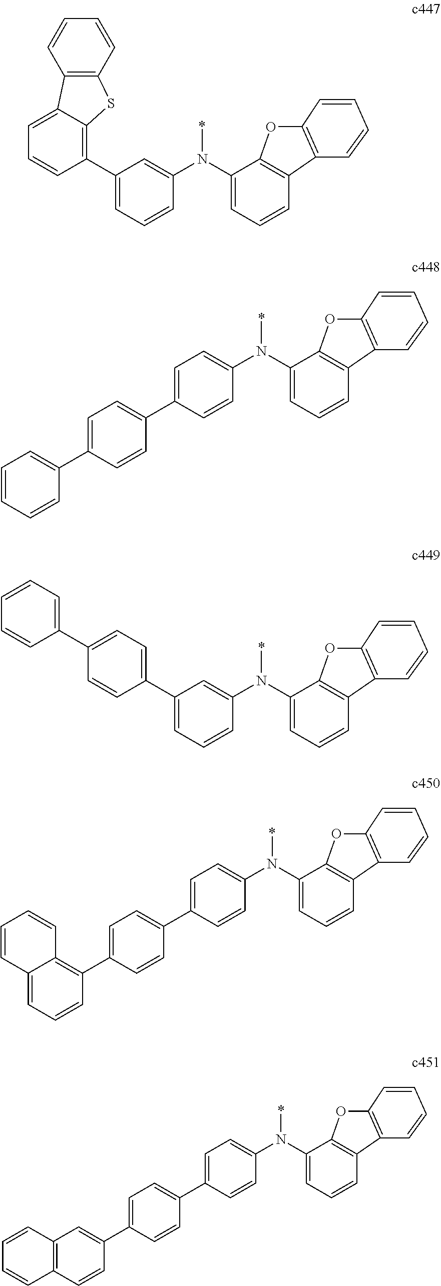 [SMILES: *N(c1cccc(-c2cccc3c2sc2ccccc23)c1)c1cccc2c1oc1ccccc12 ; *N(c1ccc(-c2ccc(-c3ccccc3)cc2)cc1)c1cccc2c1oc1ccccc12 ; *N(c1cccc(-c2ccc(-c3ccccc3)cc2)c1)c1cccc2c1oc1ccccc12 ; *N(c1ccc(-c2ccc(-c3cccc4ccccc34)cc2)cc1)c1cccc2c1oc1ccccc12 ; *N(c1ccc(-c2ccc(-c3ccc4ccccc4c3)cc2)cc1)c1cccc2c1oc1ccccc12]

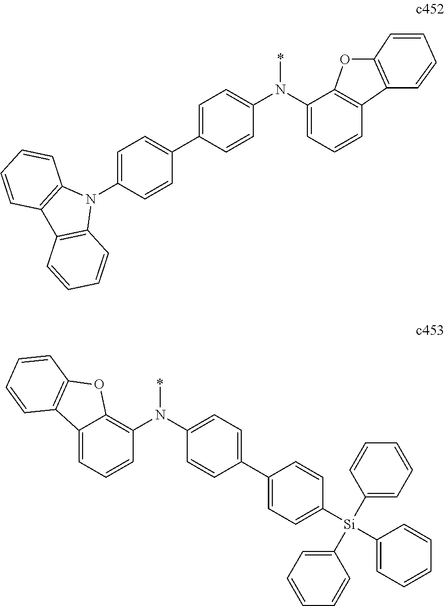 [SMILES: *N(c1ccc(-c2ccc(-n3c4ccccc4c4ccccc43)cc2)cc1)c1cccc2c1oc1ccccc12 ; *N(c1ccc(-c2ccc([Si](c3ccccc3)(c3ccccc3)c3ccccc3)cc2)cc1)c1cccc2c1oc1ccccc12]

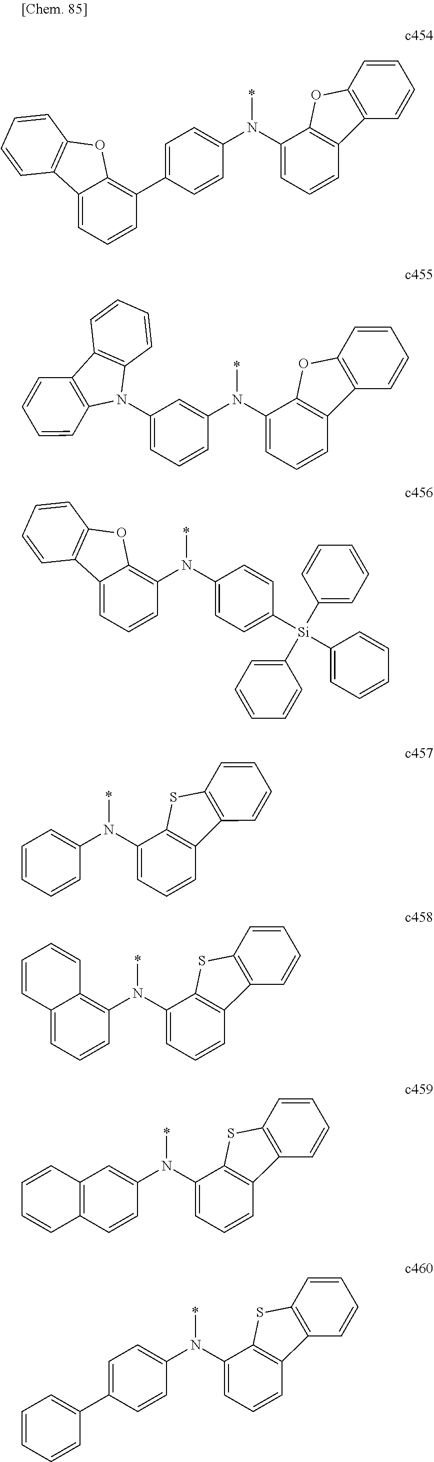

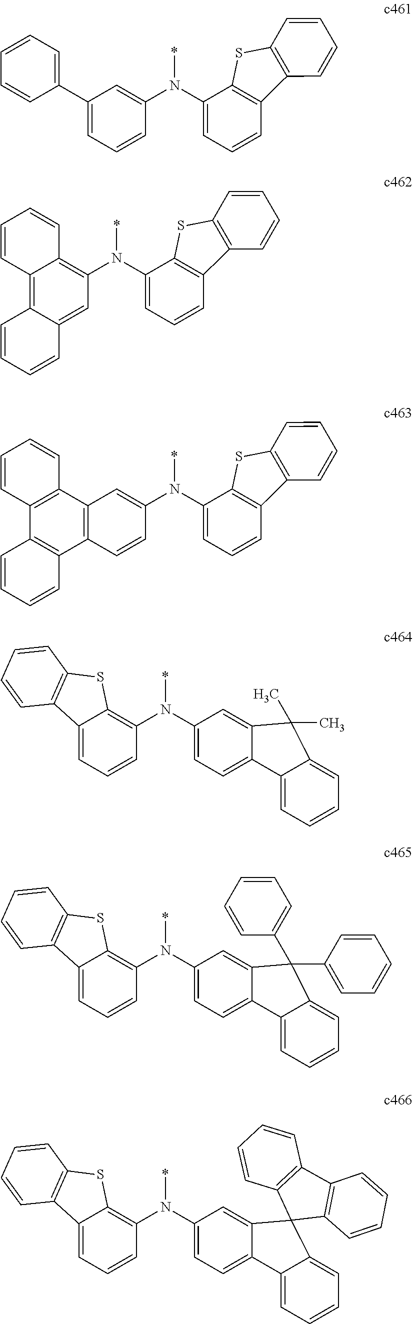

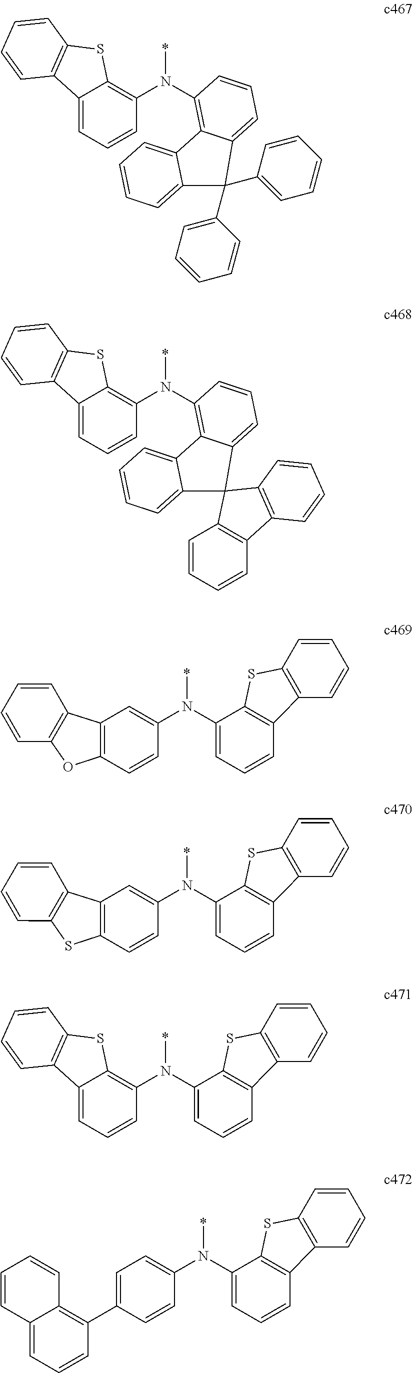 [SMILES: *N(c1cccc2c1-c1ccccc1C2(c1ccccc1)c1ccccc1)c1cccc2c1sc1ccccc12 ; *N(c1cccc2c1-c1ccccc1C21c2ccccc2-c2ccccc21)c1cccc2c1sc1ccccc12 ; *N(c1ccc2oc3ccccc3c2c1)c1cccc2c1sc1ccccc12 ; *N(c1ccc2sc3ccccc3c2c1)c1cccc2c1sc1ccccc12 ; *N(c1cccc2c1sc1ccccc12)c1cccc2c1sc1ccccc12 ; *N(c1ccc(-c2cccc3ccccc23)cc1)c1cccc2c1sc1ccccc12]

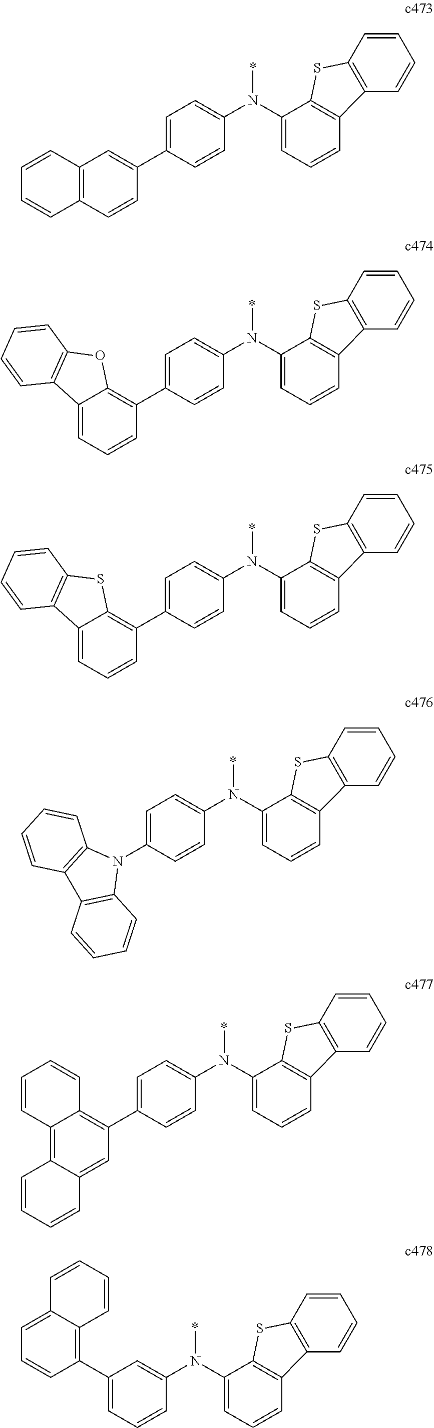

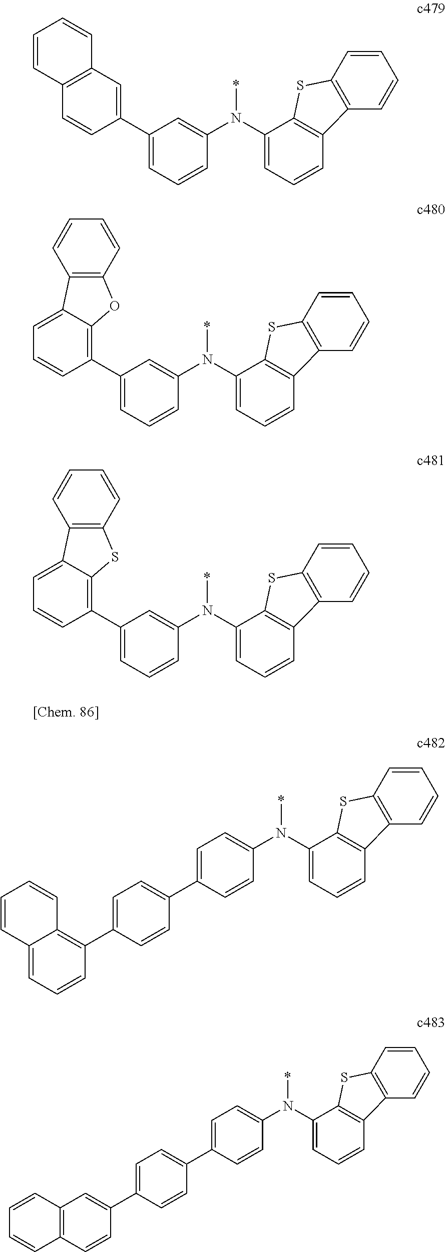 [SMILES: *N(c1cccc(-c2ccc3ccccc3c2)c1)c1cccc2c1sc1ccccc12 ; *N(c1cccc(-c2cccc3c2oc2ccccc23)c1)c1cccc2c1sc1ccccc12 ; *N(c1cccc(-c2cccc3c2sc2ccccc23)c1)c1cccc2c1sc1ccccc12 ; *N(c1ccc(-c2ccc(-c3cccc4ccccc34)cc2)cc1)c1cccc2c1sc1ccccc12 ; *N(c1ccc(-c2ccc(-c3ccc4ccccc4c3)cc2)cc1)c1cccc2c1sc1ccccc12]

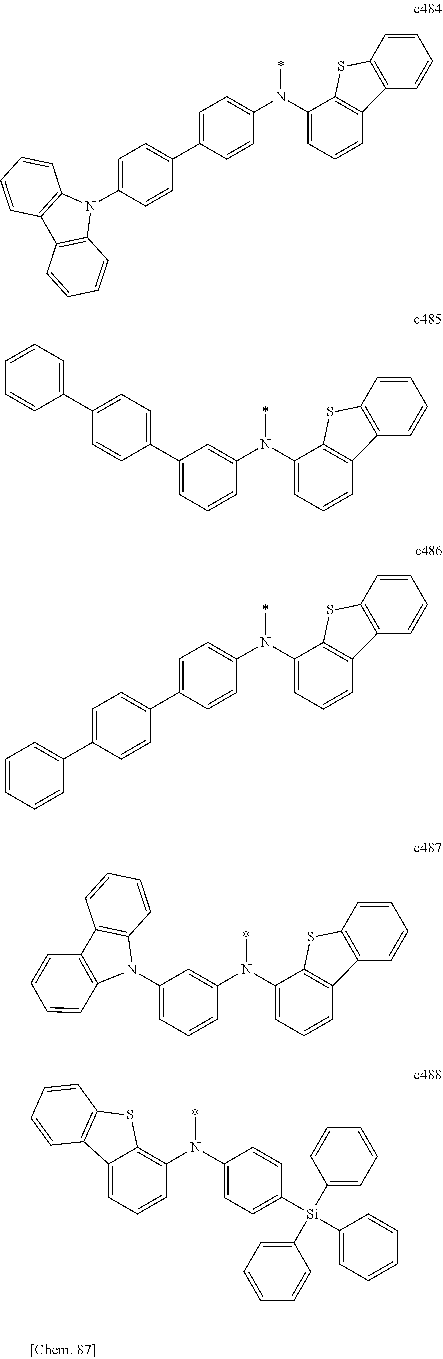 [SMILES: *N(c1ccc(-c2ccc(-n3c4ccccc4c4ccccc43)cc2)cc1)c1cccc2c1sc1ccccc12 ; *N(c1cccc(-c2ccc(-c3ccccc3)cc2)c1)c1cccc2c1sc1ccccc12 ; *N(c1ccc(-c2ccc(-c3ccccc3)cc2)cc1)c1cccc2c1sc1ccccc12 ; *N(c1cccc(-n2c3ccccc3c3ccccc32)c1)c1cccc2c1sc1ccccc12 ; *N(c1ccc([Si](c2ccccc2)(c2ccccc2)c2ccccc2)cc1)c1cccc2c1sc1ccccc12]

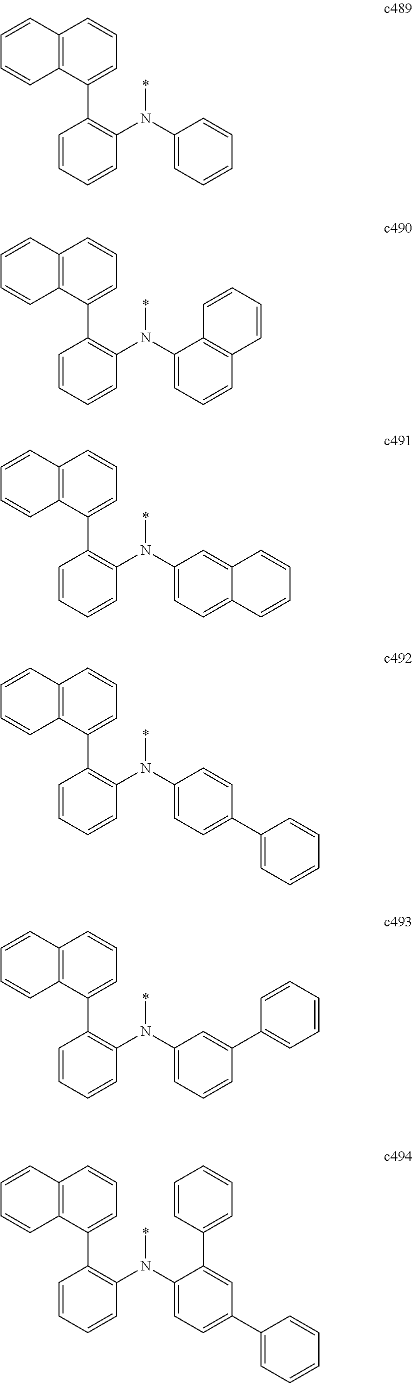

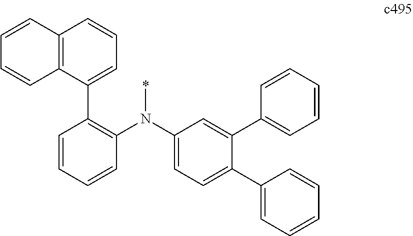 [SMILES: *N(c1ccc(-c2ccccc2)c(-c2ccccc2)c1)c1ccccc1-c1cccc2ccccc12]

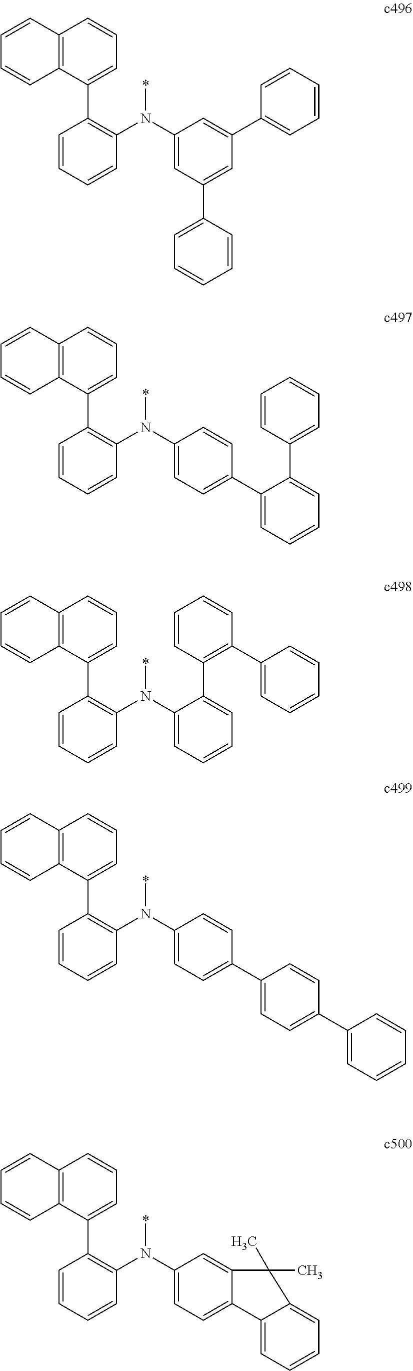 [SMILES: *N(c1cc(-c2ccccc2)cc(-c2ccccc2)c1)c1ccccc1-c1cccc2ccccc12 ; *N(c1ccc(-c2ccccc2-c2ccccc2)cc1)c1ccccc1-c1cccc2ccccc12 ; *N(c1ccccc1-c1ccccc1-c1ccccc1)c1ccccc1-c1cccc2ccccc12 ; *N(c1ccc(-c2ccc(-c3ccccc3)cc2)cc1)c1ccccc1-c1cccc2ccccc12 ; *N(c1ccc2c(c1)C(C)(C)c1ccccc1-2)c1ccccc1-c1cccc2ccccc12]

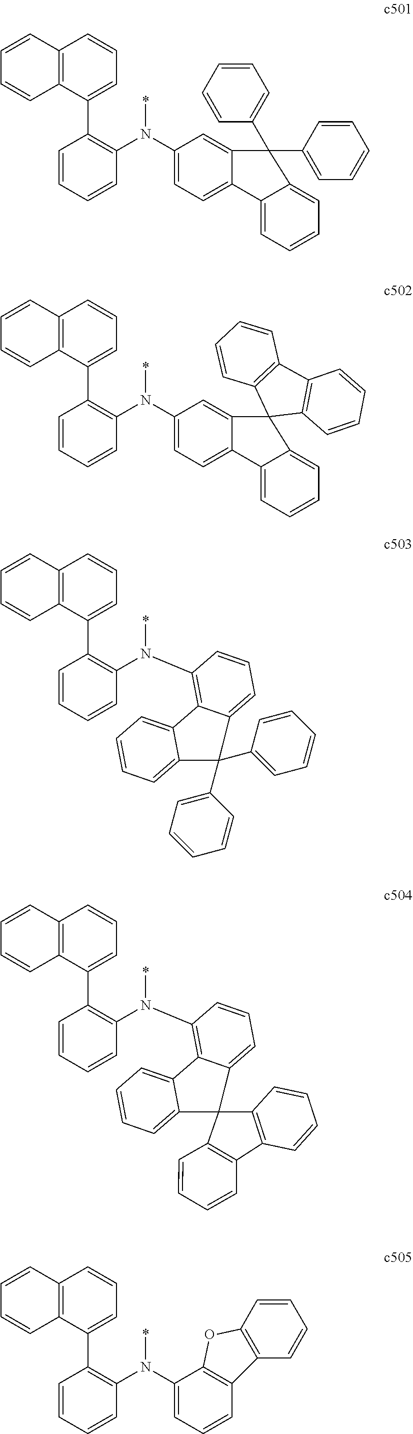 [SMILES: *N(c1ccc2c(c1)C(c1ccccc1)(c1ccccc1)c1ccccc1-2)c1ccccc1-c1cccc2ccccc12 ; *N(c1ccc2c(c1)C1(c3ccccc3-c3ccccc31)c1ccccc1-2)c1ccccc1-c1cccc2ccccc12 ; *N(c1ccccc1-c1cccc2ccccc12)c1cccc2c1-c1ccccc1C2(c1ccccc1)c1ccccc1 ; *N(c1ccccc1-c1cccc2ccccc12)c1cccc2c1-c1ccccc1C21c2ccccc2-c2ccccc21 ; *N(c1ccccc1-c1cccc2ccccc12)c1cccc2c1oc1ccccc12]

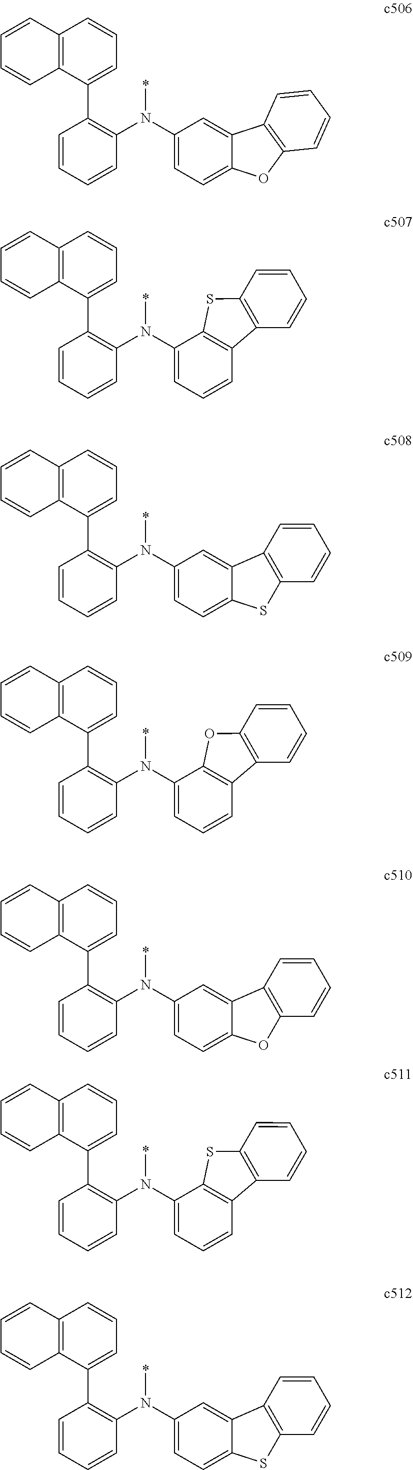

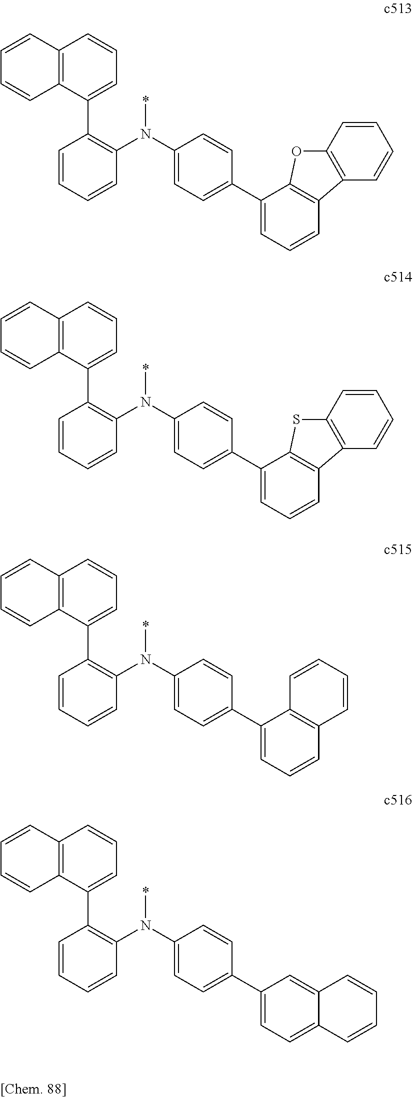 [SMILES: *N(c1ccc(-c2cccc3c2oc2ccccc23)cc1)c1ccccc1-c1cccc2ccccc12 ; *N(c1ccc(-c2cccc3c2sc2ccccc23)cc1)c1ccccc1-c1cccc2ccccc12 ; *N(c1ccc(-c2cccc3ccccc23)cc1)c1ccccc1-c1cccc2ccccc12 ; *N(c1ccc(-c2ccc3ccccc3c2)cc1)c1ccccc1-c1cccc2ccccc12]

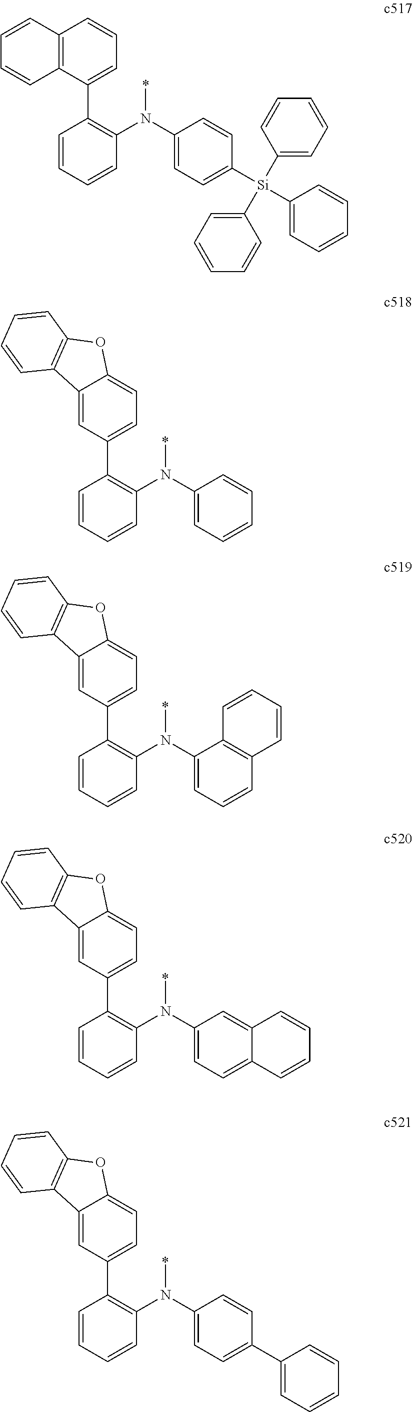 [SMILES: *N(c1ccc([Si](c2ccccc2)(c2ccccc2)c2ccccc2)cc1)c1ccccc1-c1cccc2ccccc12 ; *N(c1ccccc1)c1ccccc1-c1ccc2oc3ccccc3c2c1 ; *N(c1ccccc1-c1ccc2oc3ccccc3c2c1)c1cccc2ccccc12 ; *N(c1ccc2ccccc2c1)c1ccccc1-c1ccc2oc3ccccc3c2c1 ; *N(c1ccc(-c2ccccc2)cc1)c1ccccc1-c1ccc2oc3ccccc3c2c1]

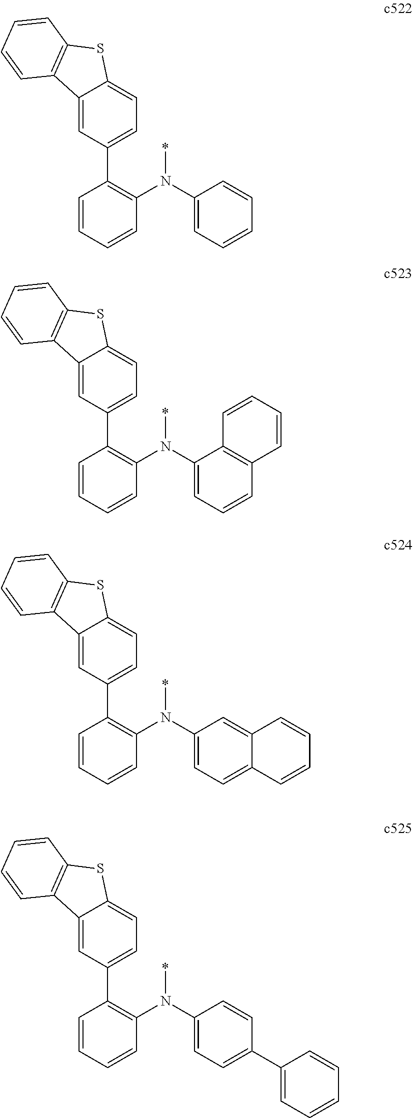 [SMILES: *N(c1ccccc1)c1ccccc1-c1ccc2sc3ccccc3c2c1 ; *N(c1ccccc1-c1ccc2sc3ccccc3c2c1)c1cccc2ccccc12 ; *N(c1ccc2ccccc2c1)c1ccccc1-c1ccc2sc3ccccc3c2c1 ; *N(c1ccc(-c2ccccc2)cc1)c1ccccc1-c1ccc2sc3ccccc3c2c1]

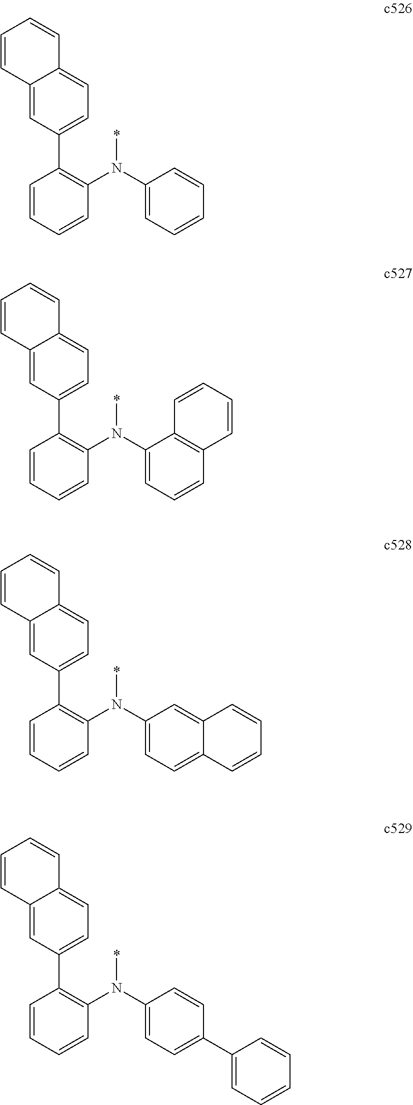 [SMILES: *N(c1ccccc1)c1ccccc1-c1ccc2ccccc2c1 ; *N(c1ccccc1-c1ccc2ccccc2c1)c1cccc2ccccc12 ; *N(c1ccc2ccccc2c1)c1ccccc1-c1ccc2ccccc2c1 ; *N(c1ccc(-c2ccccc2)cc1)c1ccccc1-c1ccc2ccccc2c1]

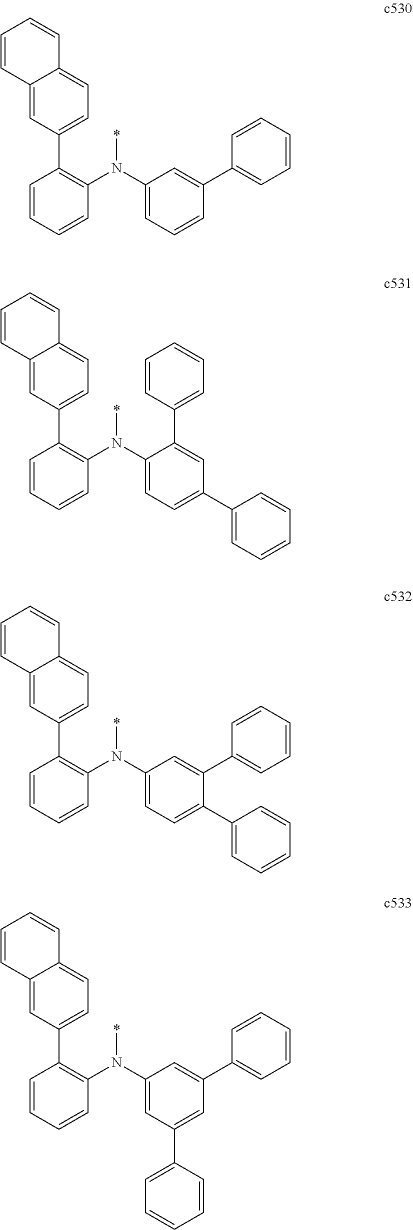 [SMILES: *N(c1cccc(-c2ccccc2)c1)c1ccccc1-c1ccc2ccccc2c1 ; *N(c1ccccc1-c1ccc2ccccc2c1)c1ccc(-c2ccccc2)cc1-c1ccccc1 ; *N(c1ccc(-c2ccccc2)c(-c2ccccc2)c1)c1ccccc1-c1ccc2ccccc2c1 ; *N(c1cc(-c2ccccc2)cc(-c2ccccc2)c1)c1ccccc1-c1ccc2ccccc2c1]

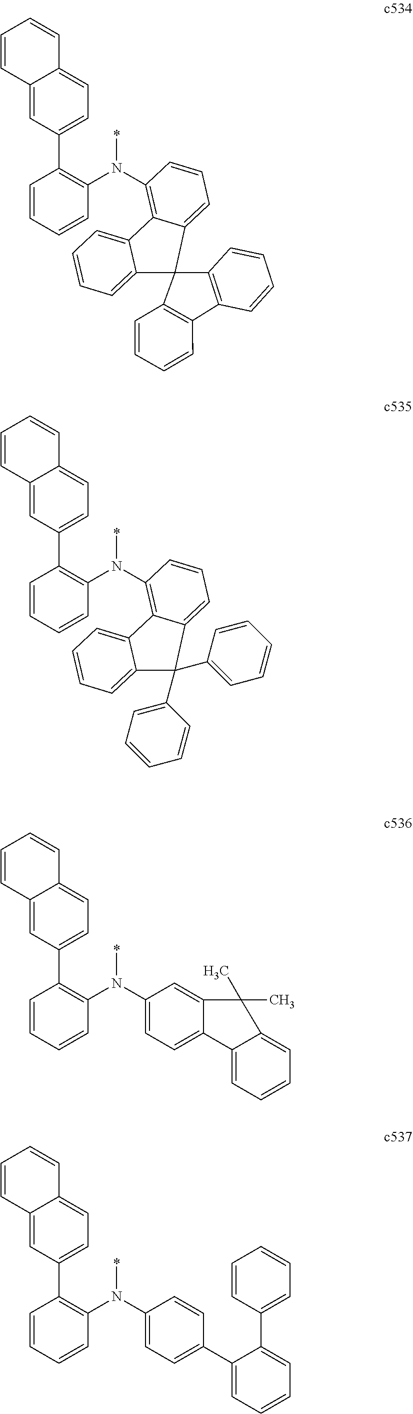 [SMILES: *N(c1ccccc1-c1ccc2ccccc2c1)c1cccc2c1-c1ccccc1C21c2ccccc2-c2ccccc21 ; *N(c1ccccc1-c1ccc2ccccc2c1)c1cccc2c1-c1ccccc1C2(c1ccccc1)c1ccccc1 ; *N(c1ccc2c(c1)C(C)(C)c1ccccc1-2)c1ccccc1-c1ccc2ccccc2c1 ; *N(c1ccc(-c2ccccc2-c2ccccc2)cc1)c1ccccc1-c1ccc2ccccc2c1]

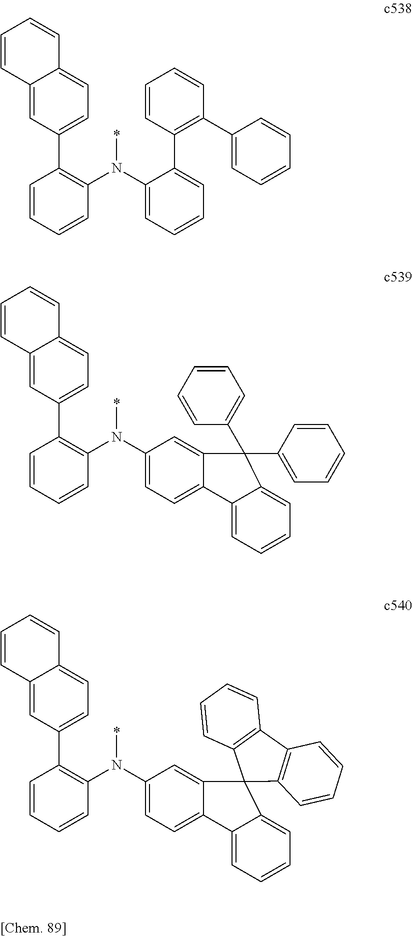 [SMILES: *N(c1ccccc1-c1ccc2ccccc2c1)c1ccccc1-c1ccccc1-c1ccccc1 ; *N(c1ccc2c(c1)C(c1ccccc1)(c1ccccc1)c1ccccc1-2)c1ccccc1-c1ccc2ccccc2c1 ; *N(c1ccc2c(c1)C1(c3ccccc3-c3ccccc31)c1ccccc1-2)c1ccccc1-c1ccc2ccccc2c1]

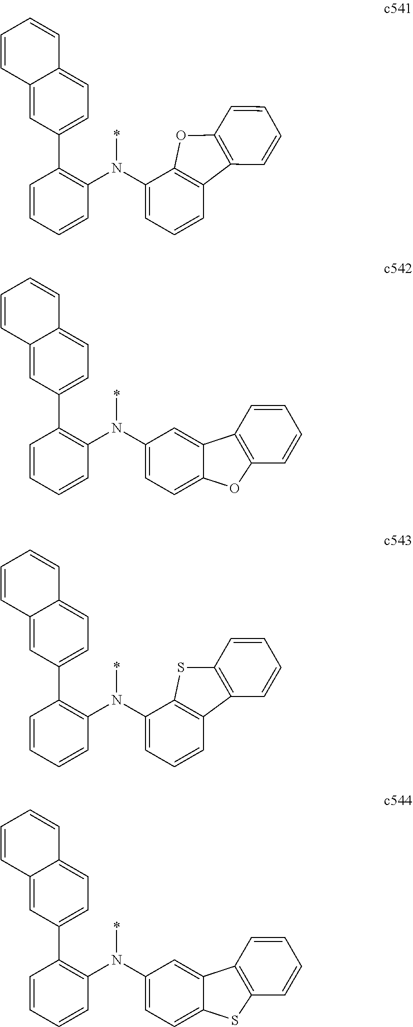 [SMILES: *N(c1ccccc1-c1ccc2ccccc2c1)c1cccc2c1oc1ccccc12 ; *N(c1ccc2oc3ccccc3c2c1)c1ccccc1-c1ccc2ccccc2c1 ; *N(c1ccccc1-c1ccc2ccccc2c1)c1cccc2c1sc1ccccc12 ; *N(c1ccc2sc3ccccc3c2c1)c1ccccc1-c1ccc2ccccc2c1]

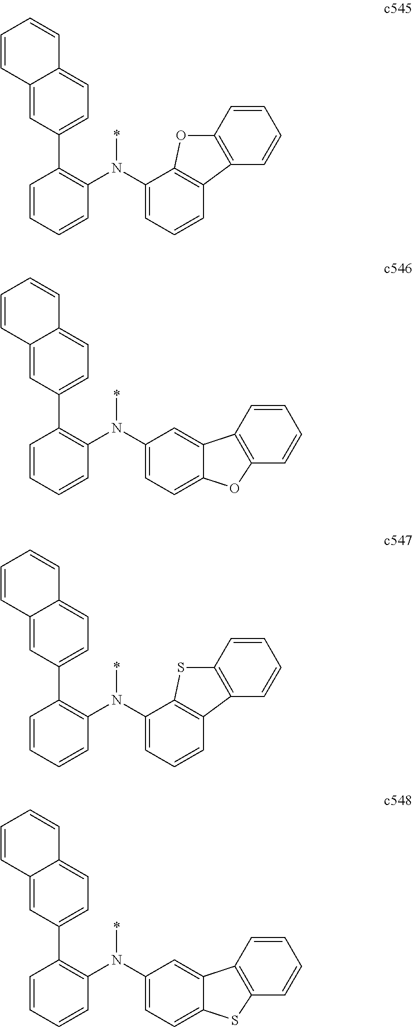 [SMILES: *N(c1ccccc1-c1ccc2ccccc2c1)c1cccc2c1oc1ccccc12 ; *N(c1ccc2oc3ccccc3c2c1)c1ccccc1-c1ccc2ccccc2c1 ; *N(c1ccccc1-c1ccc2ccccc2c1)c1cccc2c1sc1ccccc12 ; *N(c1ccc2sc3ccccc3c2c1)c1ccccc1-c1ccc2ccccc2c1]

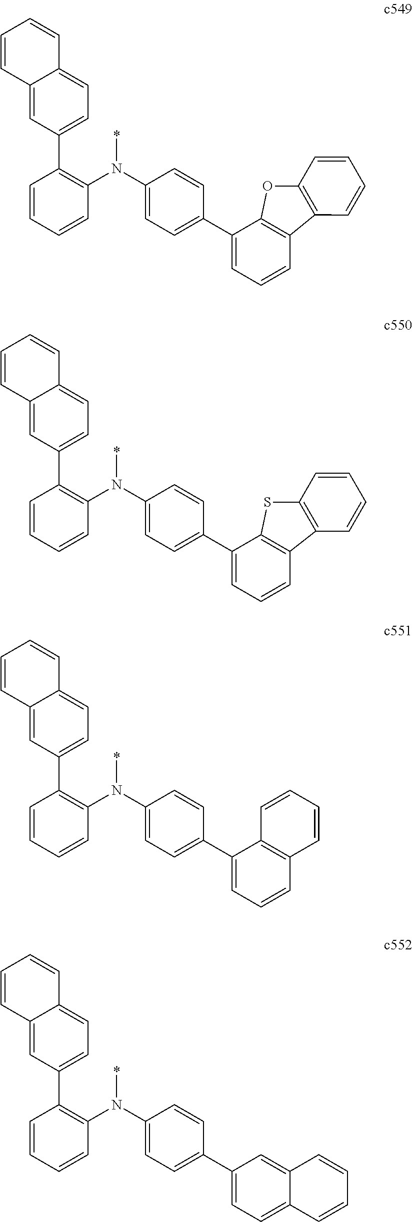 [SMILES: *N(c1ccc(-c2cccc3c2oc2ccccc23)cc1)c1ccccc1-c1ccc2ccccc2c1 ; *N(c1ccc(-c2cccc3c2sc2ccccc23)cc1)c1ccccc1-c1ccc2ccccc2c1 ; *N(c1ccc(-c2cccc3ccccc23)cc1)c1ccccc1-c1ccc2ccccc2c1 ; *N(c1ccc(-c2ccc3ccccc3c2)cc1)c1ccccc1-c1ccc2ccccc2c1]

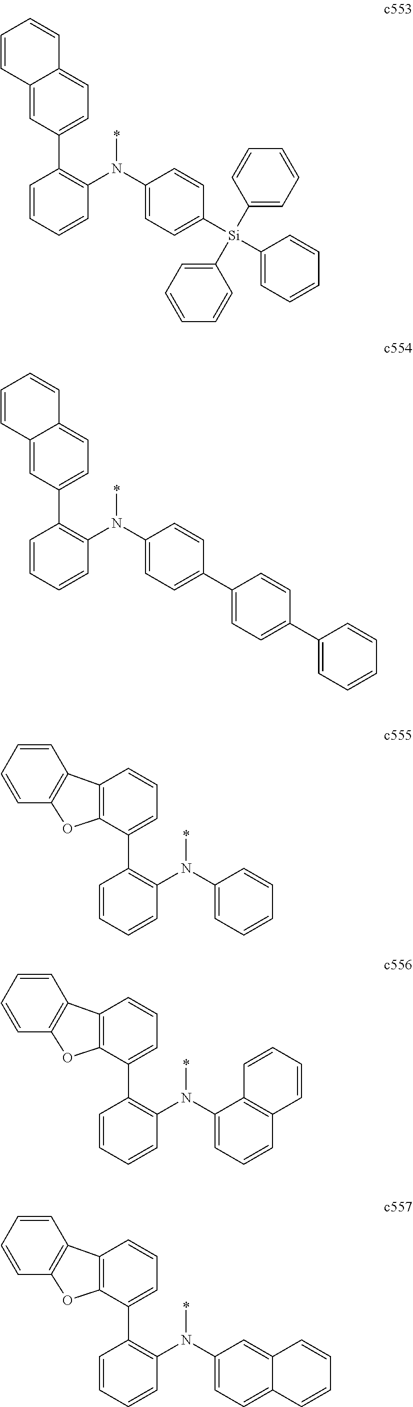 [SMILES: *N(c1ccc([Si](c2ccccc2)(c2ccccc2)c2ccccc2)cc1)c1ccccc1-c1ccc2ccccc2c1 ; *N(c1ccc(-c2ccc(-c3ccccc3)cc2)cc1)c1ccccc1-c1ccc2ccccc2c1 ; *N(c1ccccc1)c1ccccc1-c1cccc2c1oc1ccccc12 ; *N(c1ccccc1-c1cccc2c1oc1ccccc12)c1cccc2ccccc12 ; *N(c1ccc2ccccc2c1)c1ccccc1-c1cccc2c1oc1ccccc12]

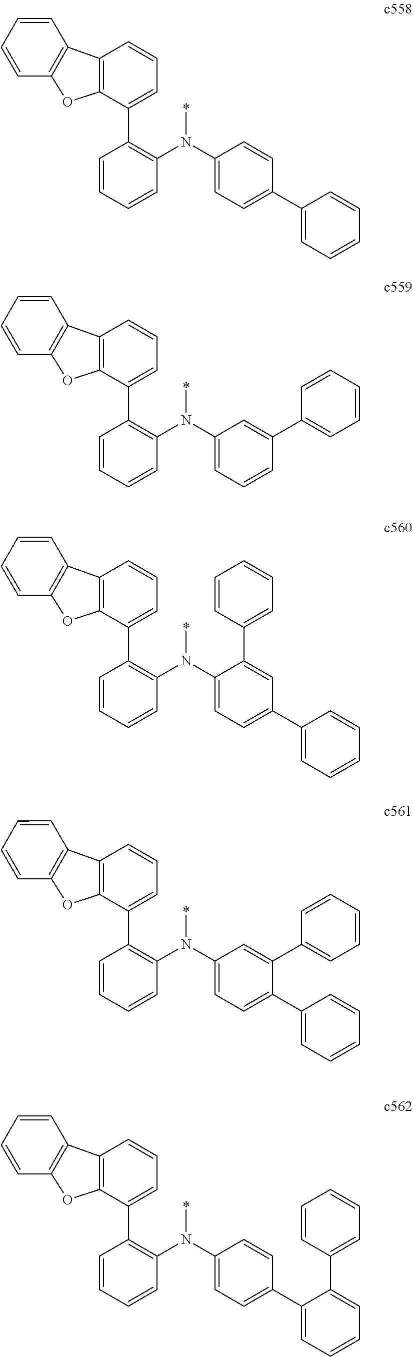 [SMILES: *N(c1ccc(-c2ccccc2)cc1)c1ccccc1-c1cccc2c1oc1ccccc12 ; *N(c1cccc(-c2ccccc2)c1)c1ccccc1-c1cccc2c1oc1ccccc12 ; *N(c1ccc(-c2ccccc2)cc1-c1ccccc1)c1ccccc1-c1cccc2c1oc1ccccc12 ; *N(c1ccc(-c2ccccc2)c(-c2ccccc2)c1)c1ccccc1-c1cccc2c1oc1ccccc12 ; *N(c1ccc(-c2ccccc2-c2ccccc2)cc1)c1ccccc1-c1cccc2c1oc1ccccc12]

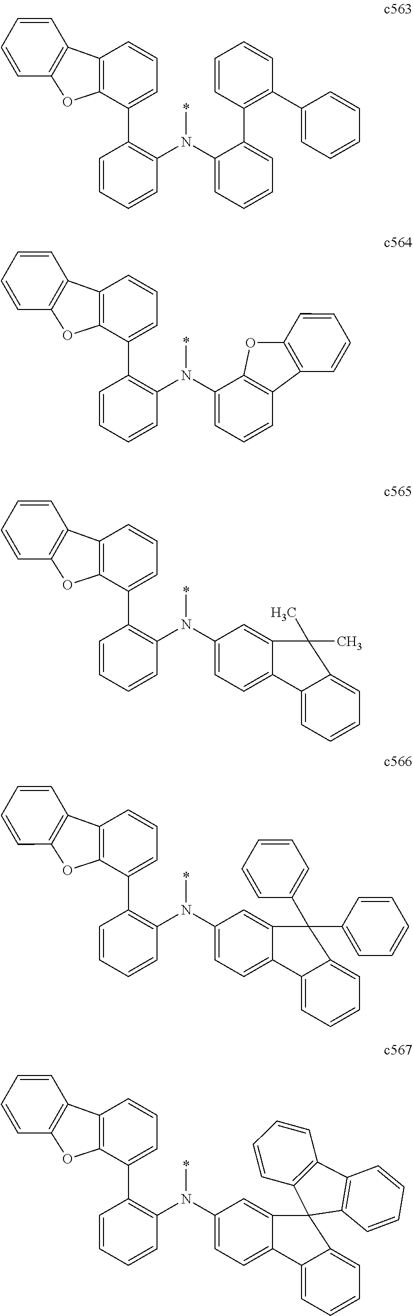 [SMILES: *N(c1ccccc1-c1ccccc1-c1ccccc1)c1ccccc1-c1cccc2c1oc1ccccc12 ; *N(c1ccccc1-c1cccc2c1oc1ccccc12)c1cccc2c1oc1ccccc12 ; *N(c1ccc2c(c1)C(C)(C)c1ccccc1-2)c1ccccc1-c1cccc2c1oc1ccccc12 ; *N(c1ccc2c(c1)C(c1ccccc1)(c1ccccc1)c1ccccc1-2)c1ccccc1-c1cccc2c1oc1ccccc12 ; *N(c1ccc2c(c1)C1(c3ccccc3-c3ccccc31)c1ccccc1-2)c1ccccc1-c1cccc2c1oc1ccccc12]

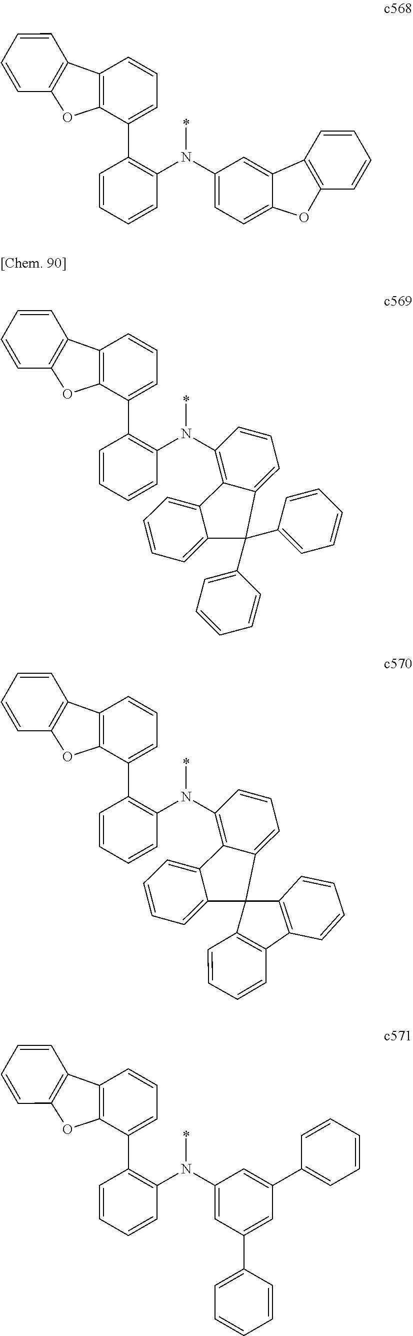 [SMILES: *N(c1ccc2oc3ccccc3c2c1)c1ccccc1-c1cccc2c1oc1ccccc12 ; *N(c1ccccc1-c1cccc2c1oc1ccccc12)c1cccc2c1-c1ccccc1C2(c1ccccc1)c1ccccc1 ; *N(c1ccccc1-c1cccc2c1oc1ccccc12)c1cccc2c1-c1ccccc1C21c2ccccc2-c2ccccc21 ; *N(c1cc(-c2ccccc2)cc(-c2ccccc2)c1)c1ccccc1-c1cccc2c1oc1ccccc12]

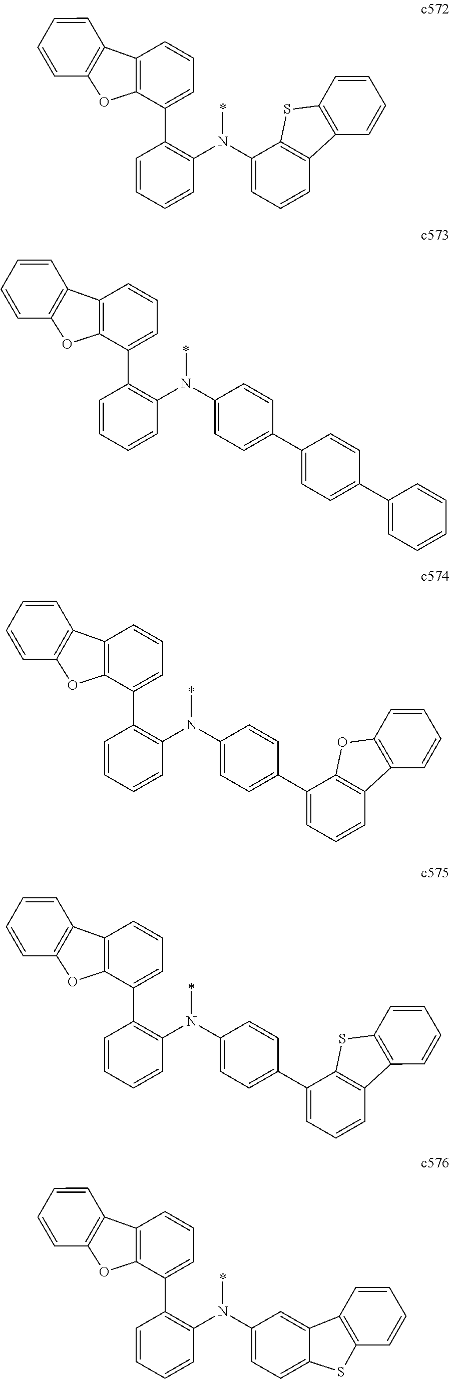 [SMILES: *N(c1ccccc1-c1cccc2c1oc1ccccc12)c1cccc2c1sc1ccccc12 ; *N(c1ccc(-c2ccc(-c3ccccc3)cc2)cc1)c1ccccc1-c1cccc2c1oc1ccccc12 ; *N(c1ccc(-c2cccc3c2oc2ccccc23)cc1)c1ccccc1-c1cccc2c1oc1ccccc12 ; *N(c1ccc(-c2cccc3c2sc2ccccc23)cc1)c1ccccc1-c1cccc2c1oc1ccccc12 ; *N(c1ccc2sc3ccccc3c2c1)c1ccccc1-c1cccc2c1oc1ccccc12]

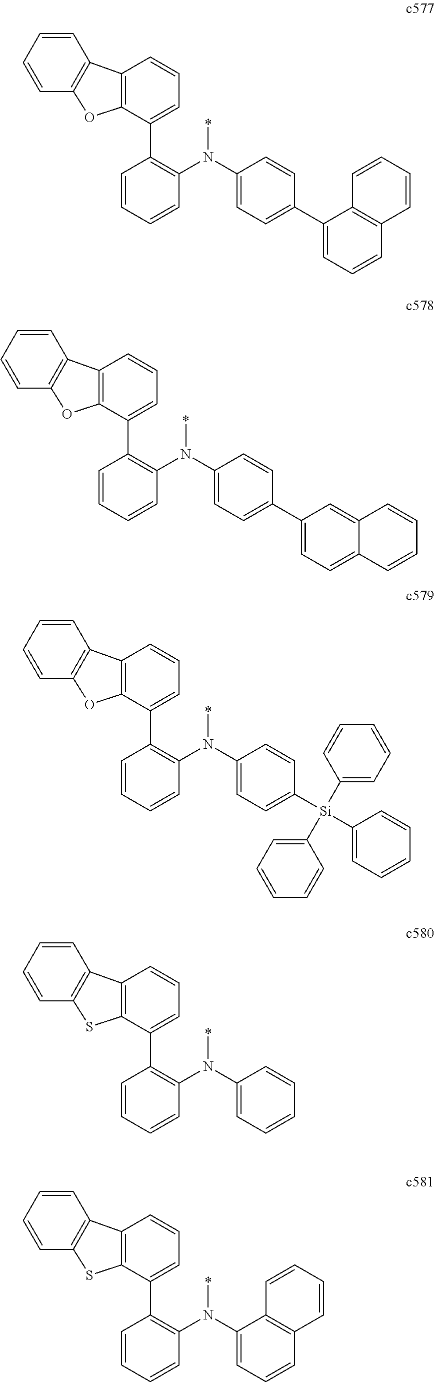 [SMILES: *N(c1ccc(-c2cccc3ccccc23)cc1)c1ccccc1-c1cccc2c1oc1ccccc12 ; *N(c1ccc(-c2ccc3ccccc3c2)cc1)c1ccccc1-c1cccc2c1oc1ccccc12 ; *N(c1ccc([Si](c2ccccc2)(c2ccccc2)c2ccccc2)cc1)c1ccccc1-c1cccc2c1oc1ccccc12 ; *N(c1ccccc1)c1ccccc1-c1cccc2c1sc1ccccc12 ; *N(c1ccccc1-c1cccc2c1sc1ccccc12)c1cccc2ccccc12]

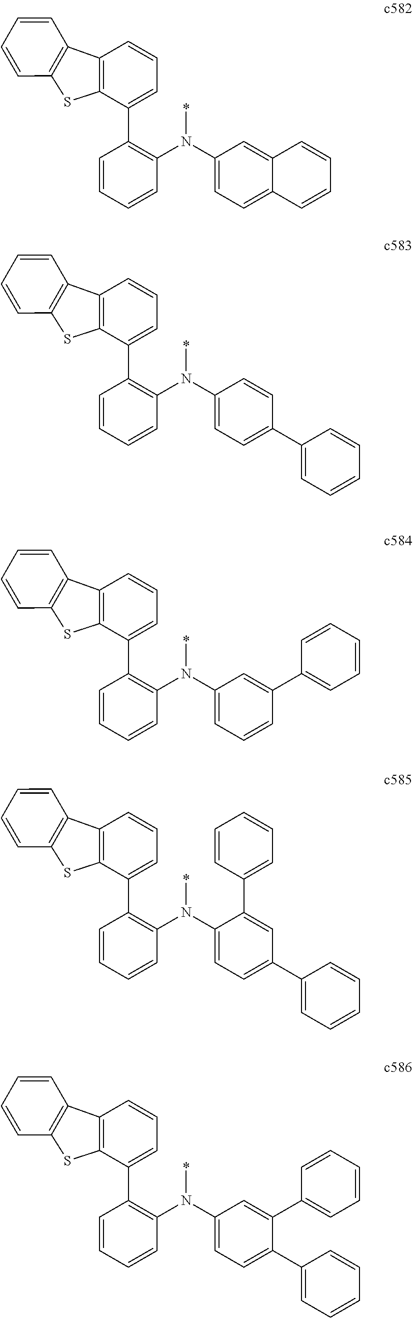 [SMILES: *N(c1ccc2ccccc2c1)c1ccccc1-c1cccc2c1sc1ccccc12 ; *N(c1ccc(-c2ccccc2)cc1)c1ccccc1-c1cccc2c1sc1ccccc12 ; *N(c1cccc(-c2ccccc2)c1)c1ccccc1-c1cccc2c1sc1ccccc12 ; *N(c1ccc(-c2ccccc2)cc1-c1ccccc1)c1ccccc1-c1cccc2c1sc1ccccc12 ; *N(c1ccc(-c2ccccc2)c(-c2ccccc2)c1)c1ccccc1-c1cccc2c1sc1ccccc12]

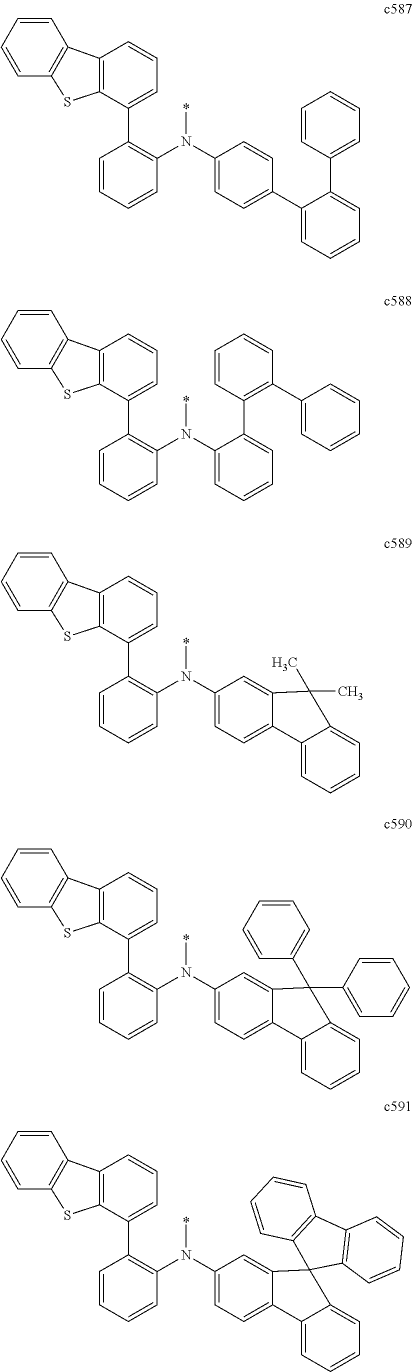 [SMILES: *N(c1ccc(-c2ccccc2-c2ccccc2)cc1)c1ccccc1-c1cccc2c1sc1ccccc12 ; *N(c1ccccc1-c1ccccc1-c1ccccc1)c1ccccc1-c1cccc2c1sc1ccccc12 ; *N(c1ccc2c(c1)C(C)(C)c1ccccc1-2)c1ccccc1-c1cccc2c1sc1ccccc12 ; *N(c1ccc2c(c1)C(c1ccccc1)(c1ccccc1)c1ccccc1-2)c1ccccc1-c1cccc2c1sc1ccccc12 ; *N(c1ccc2c(c1)C1(c3ccccc3-c3ccccc31)c1ccccc1-2)c1ccccc1-c1cccc2c1sc1ccccc12]

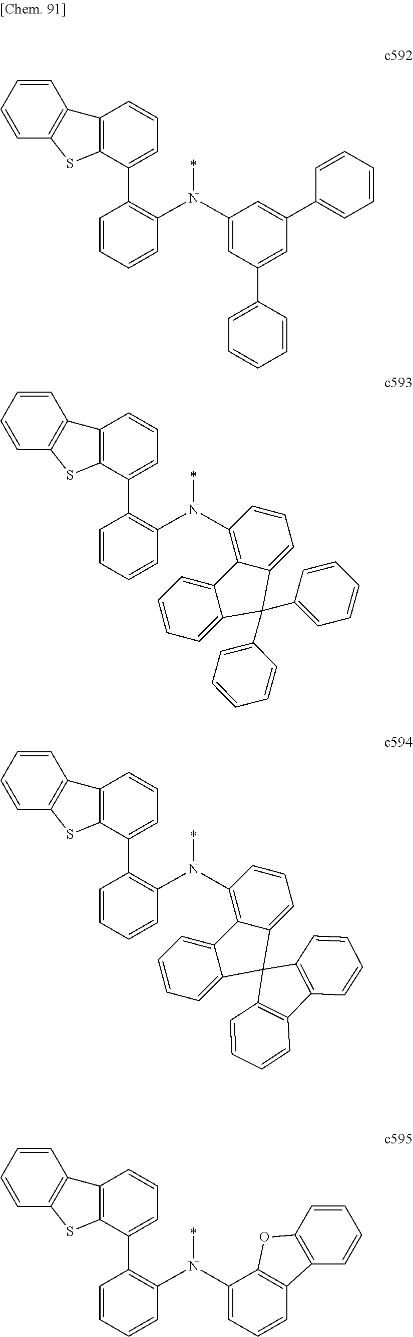

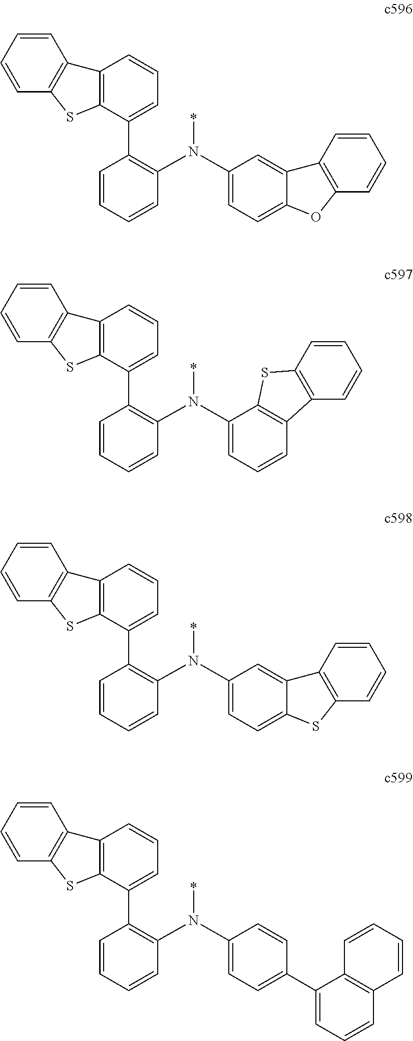

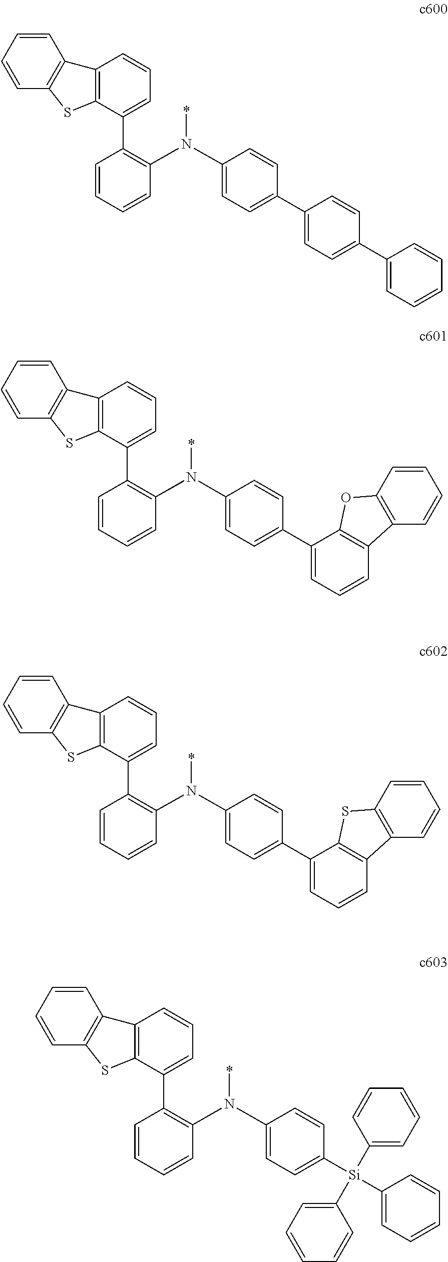

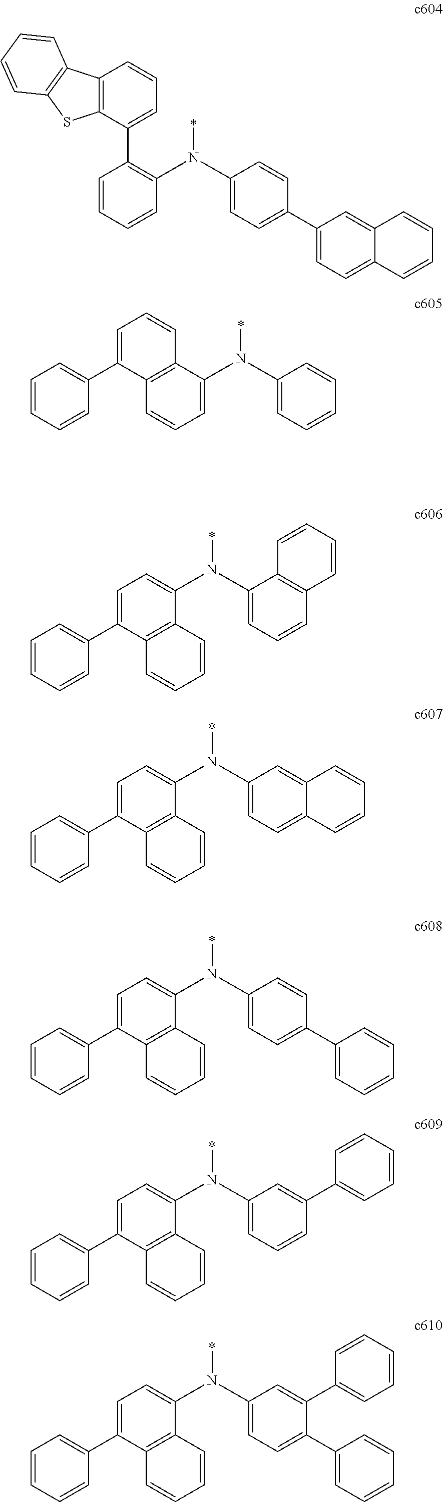

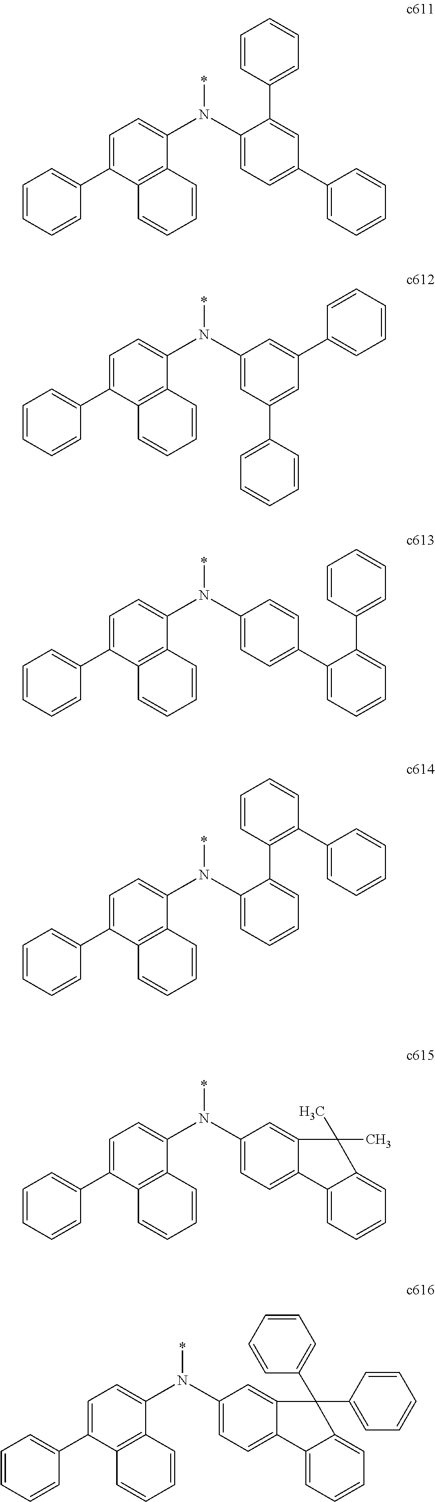

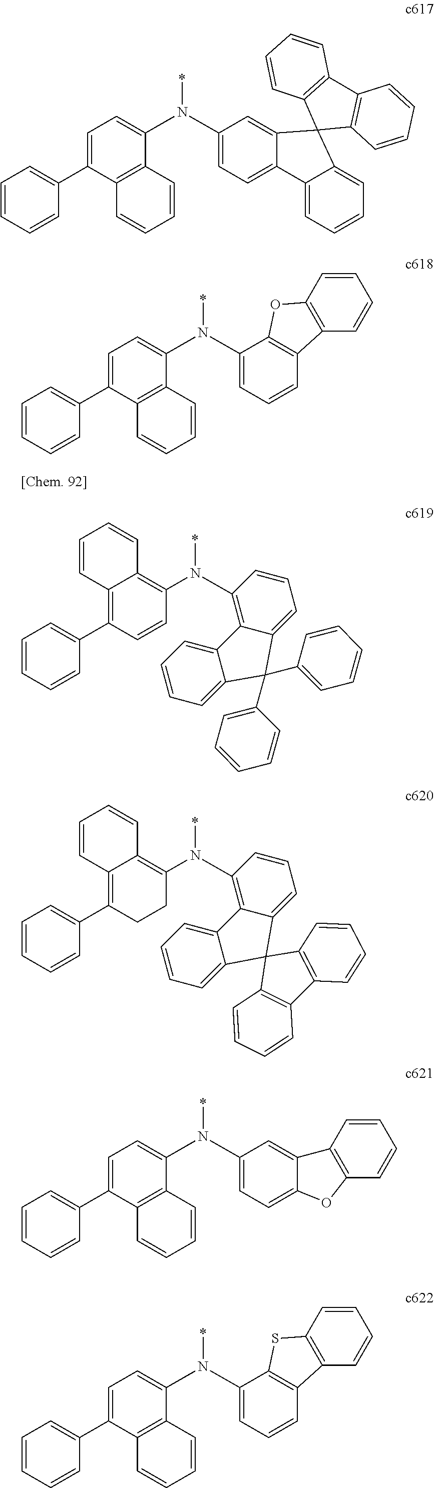

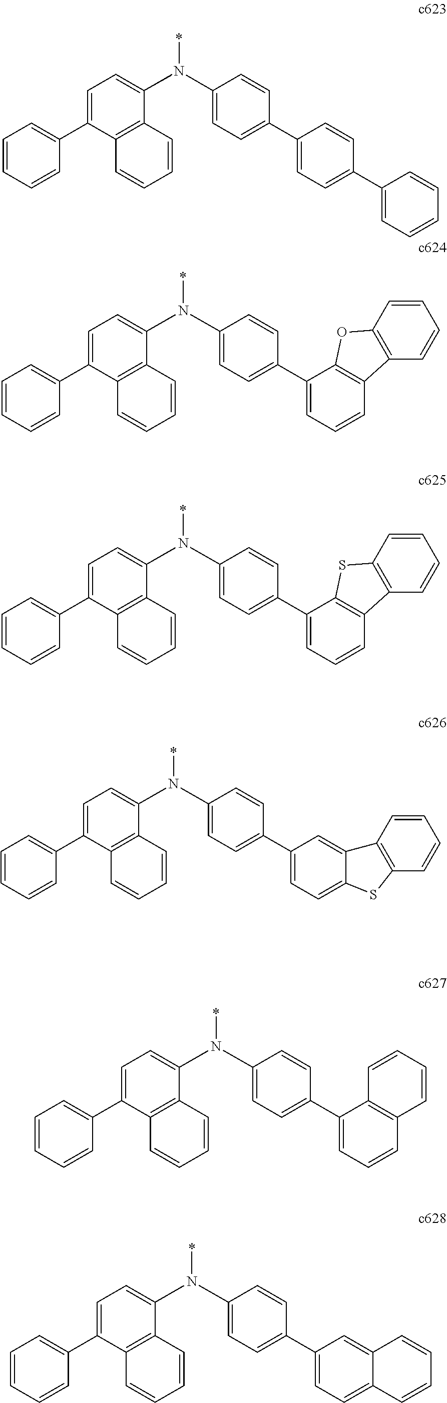

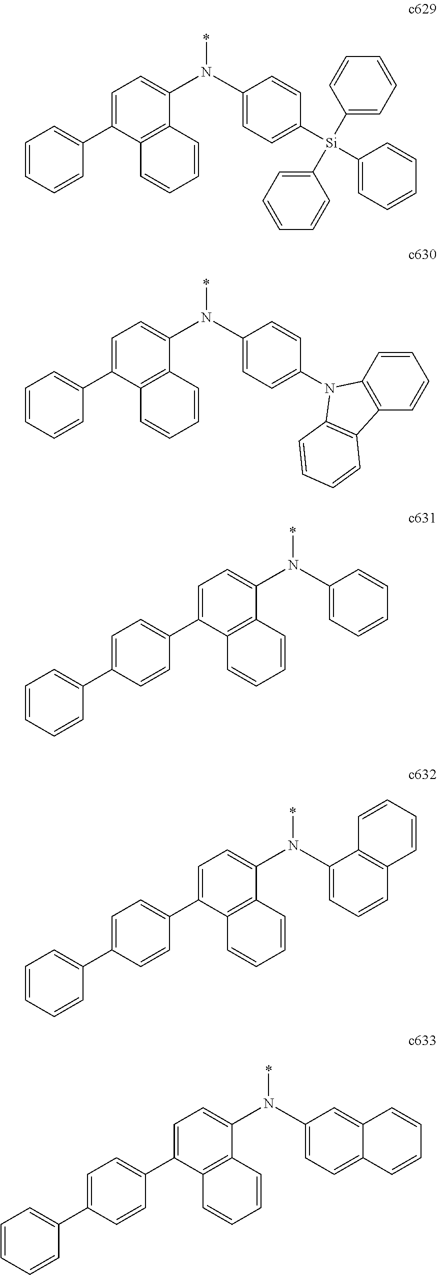

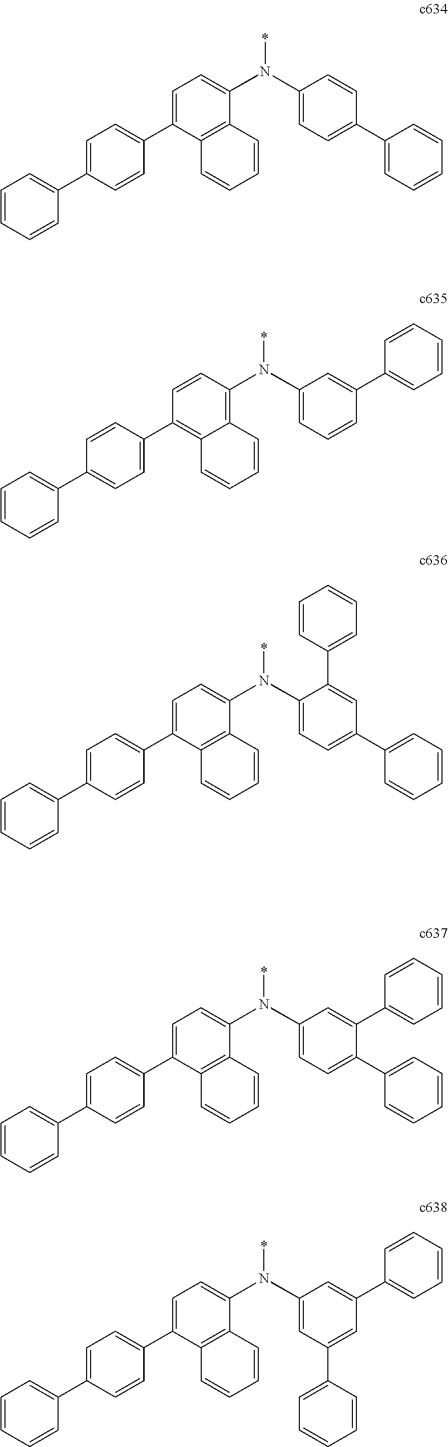

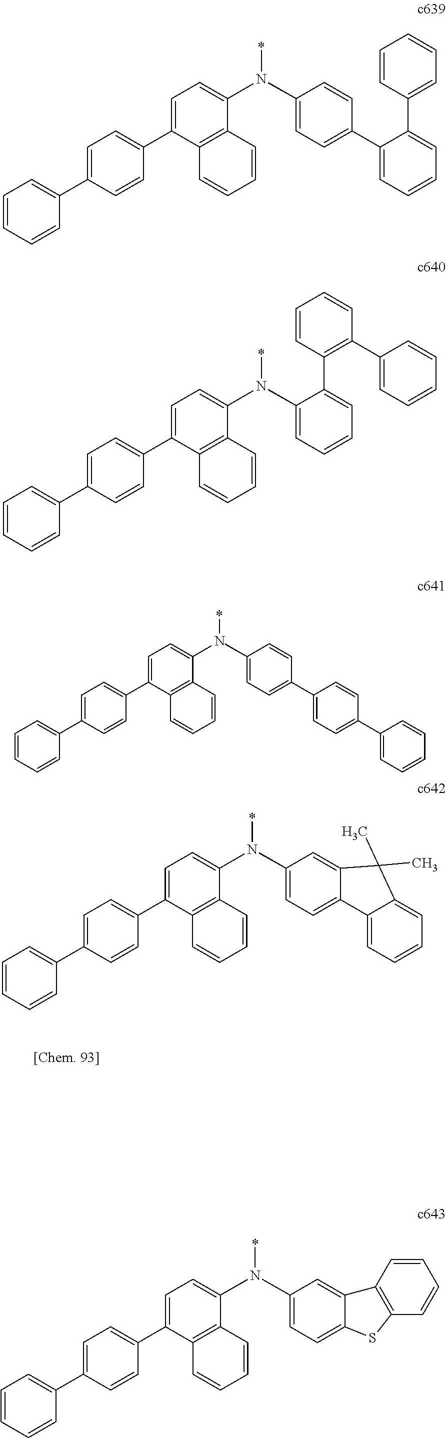

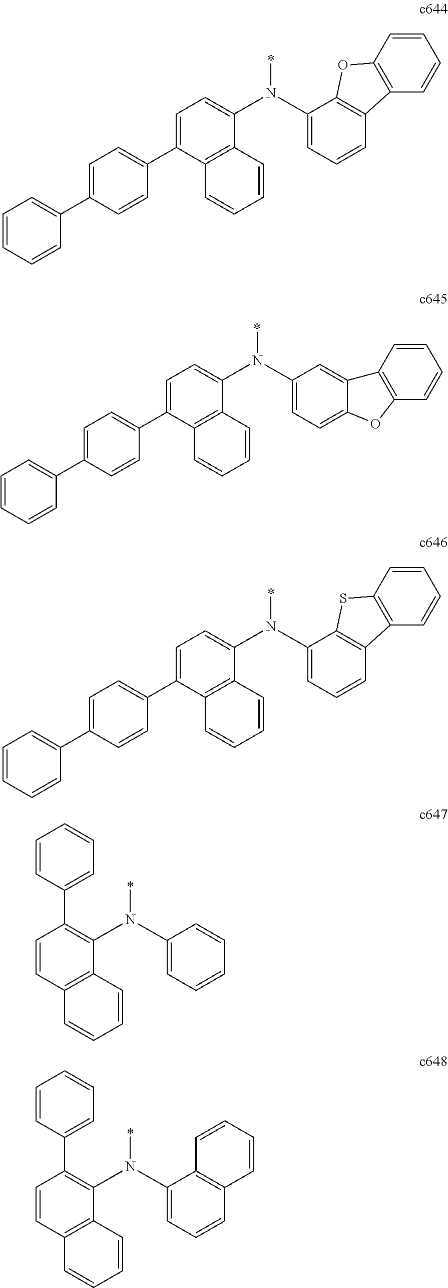

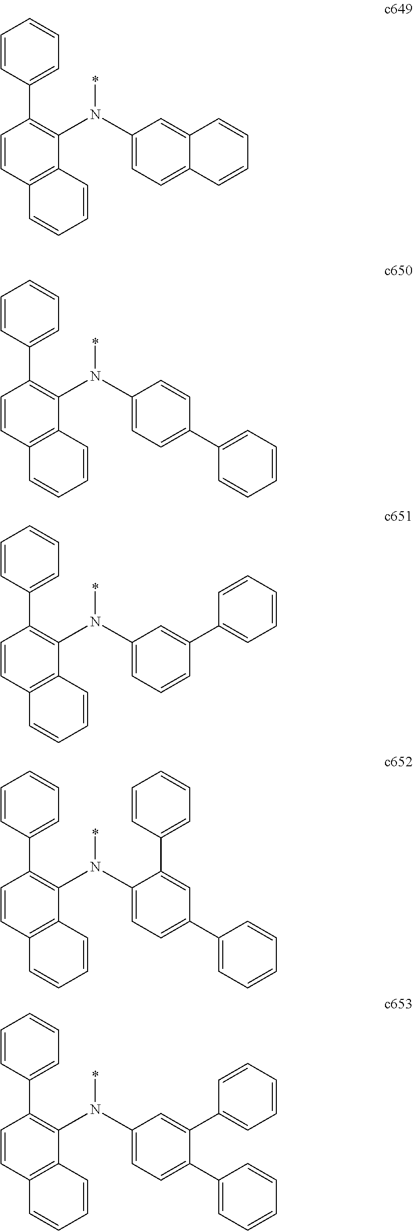

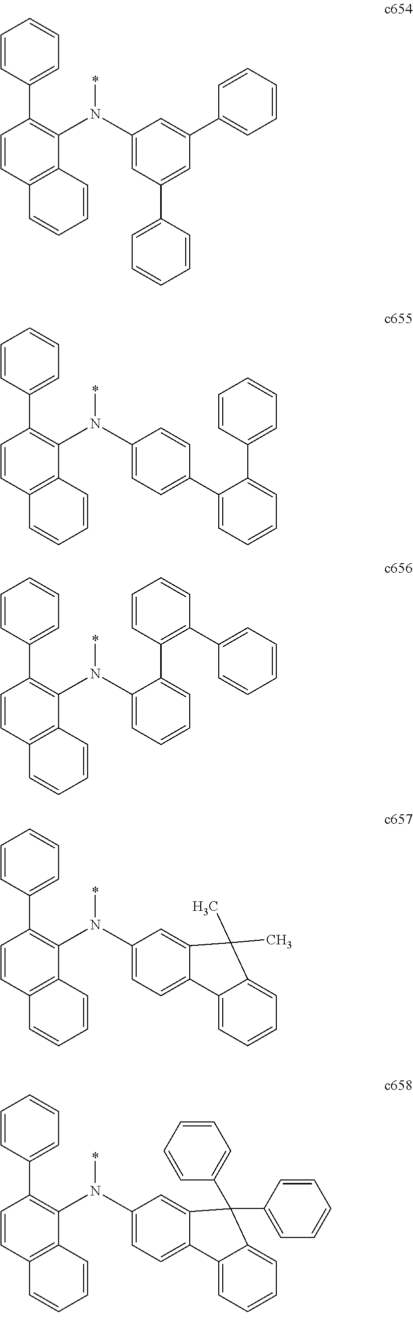

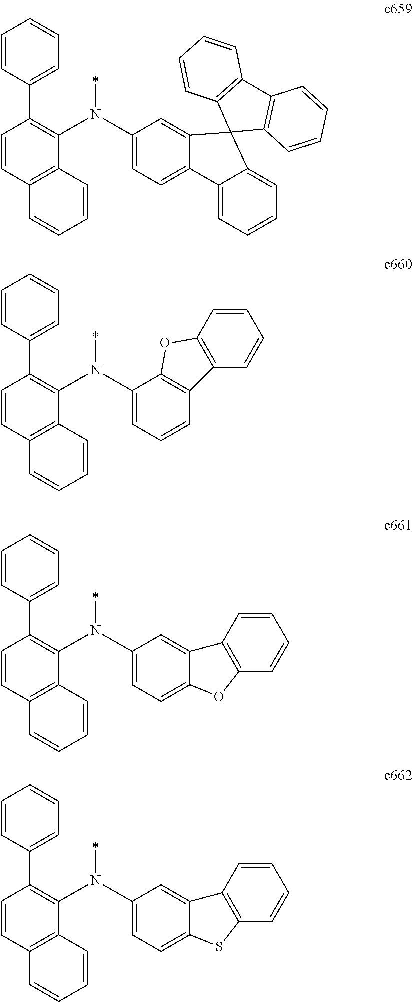

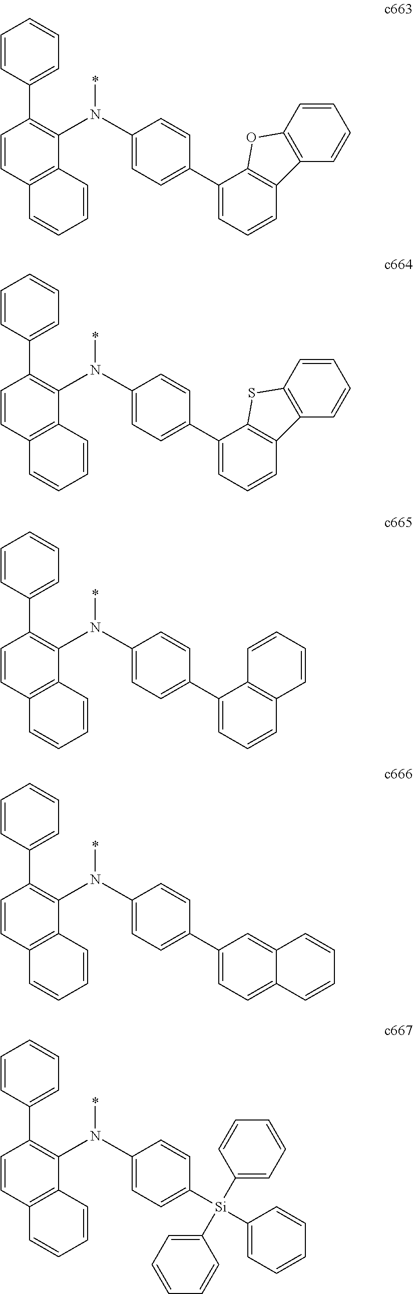

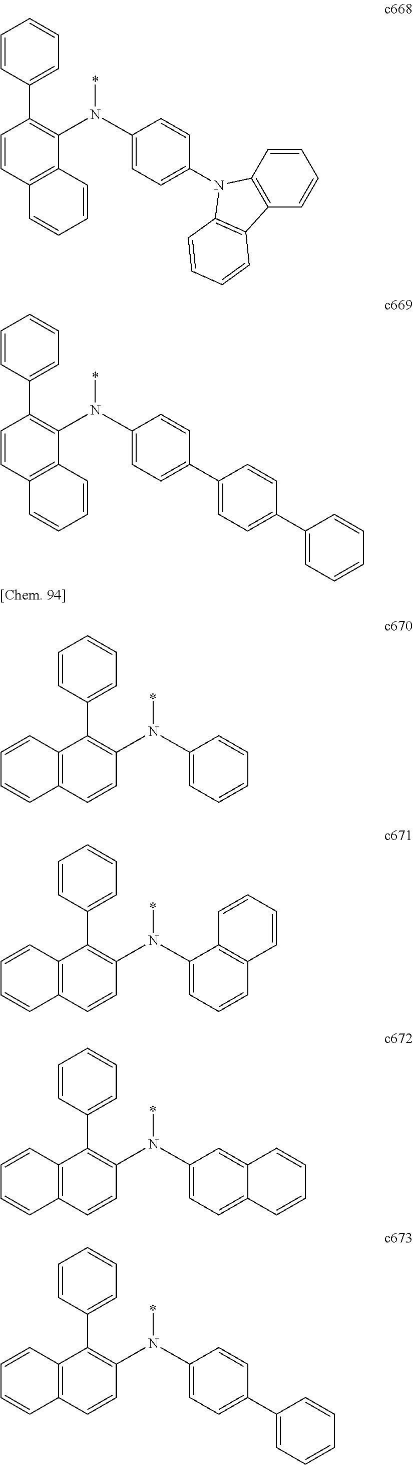

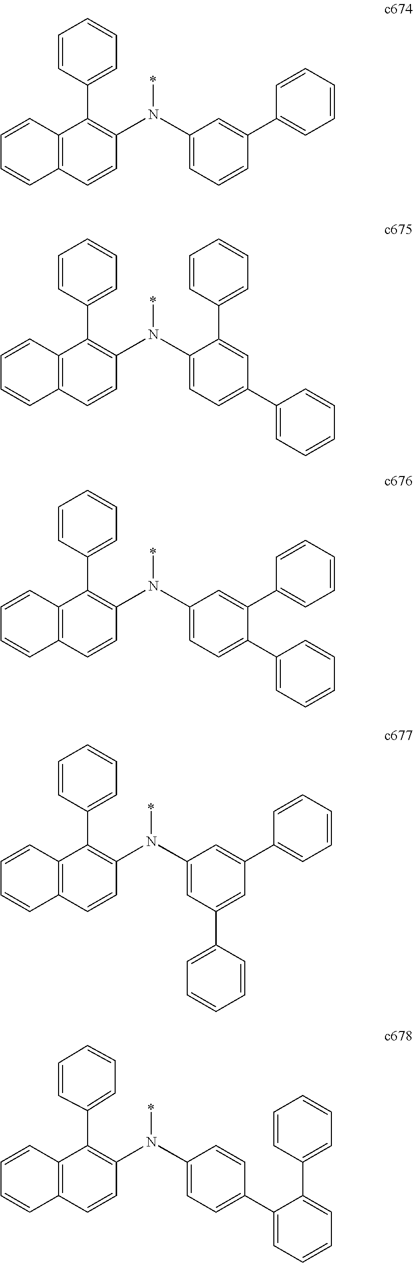

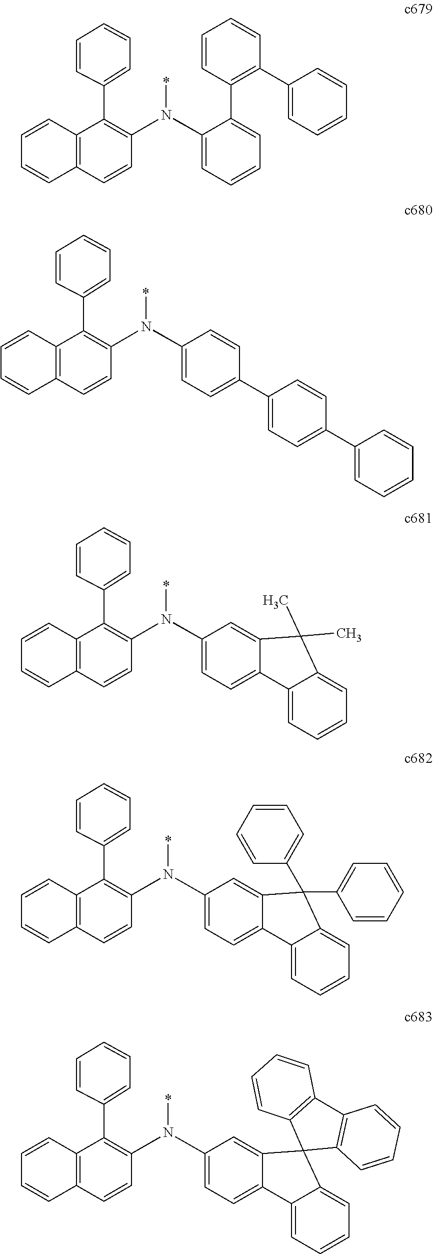

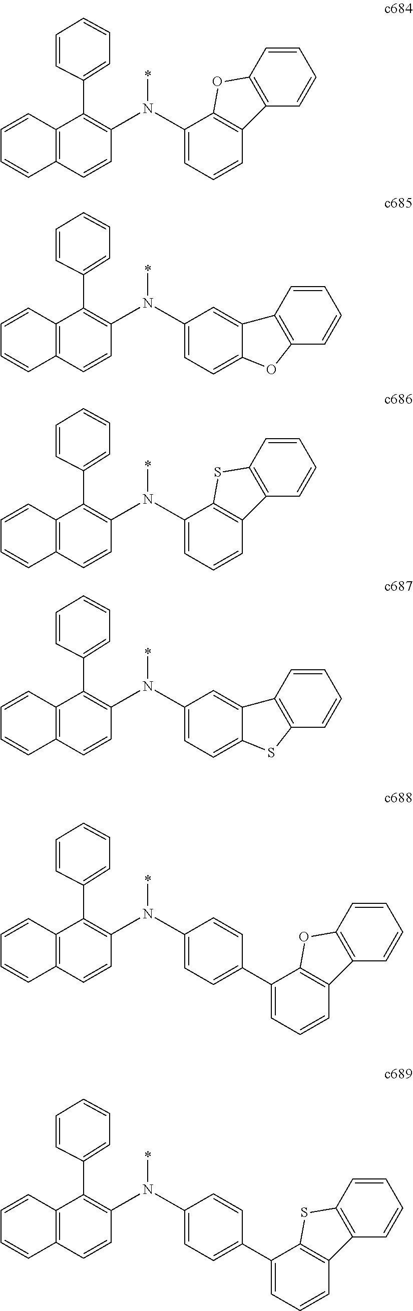

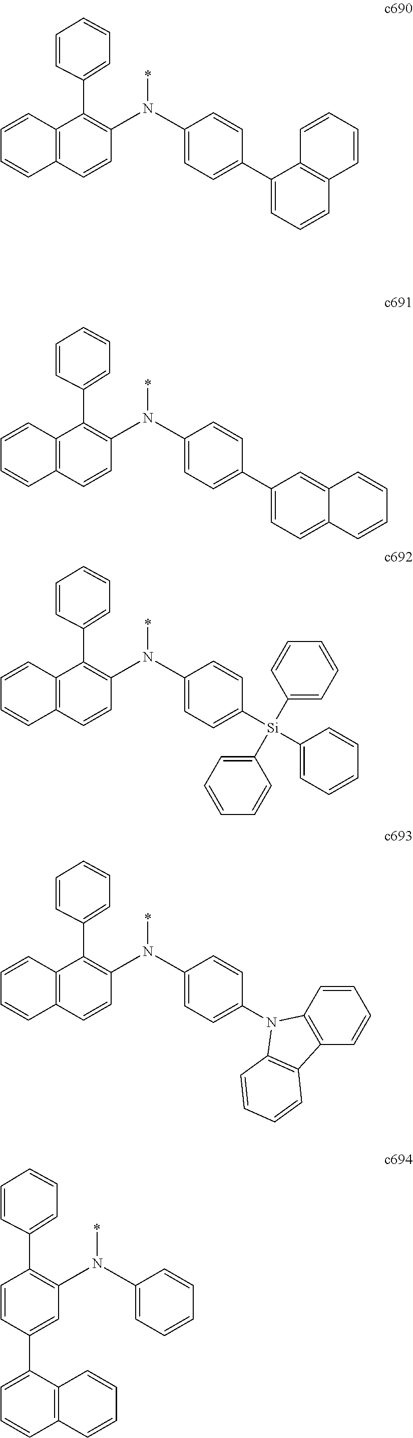

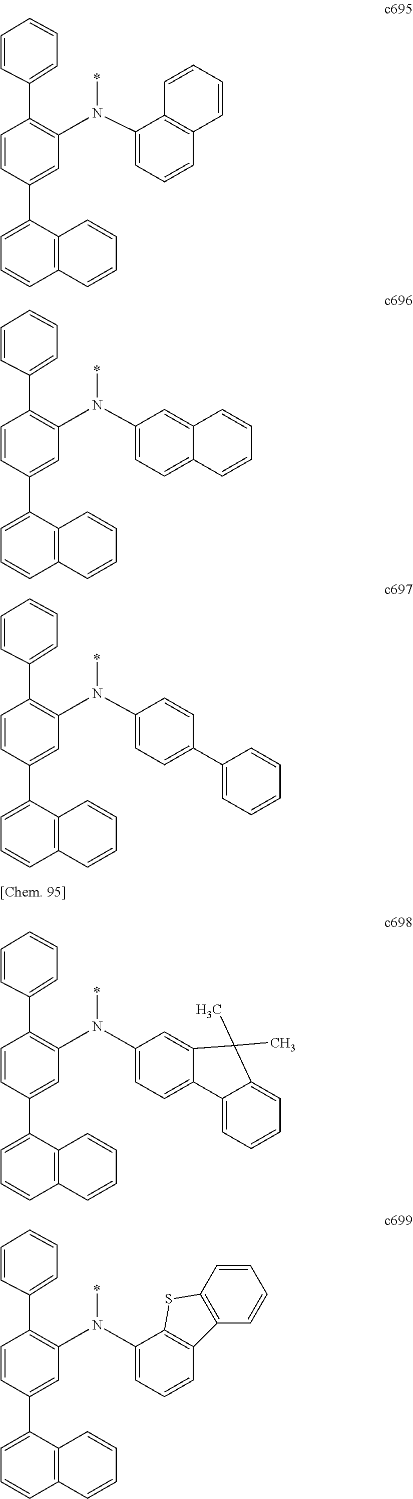

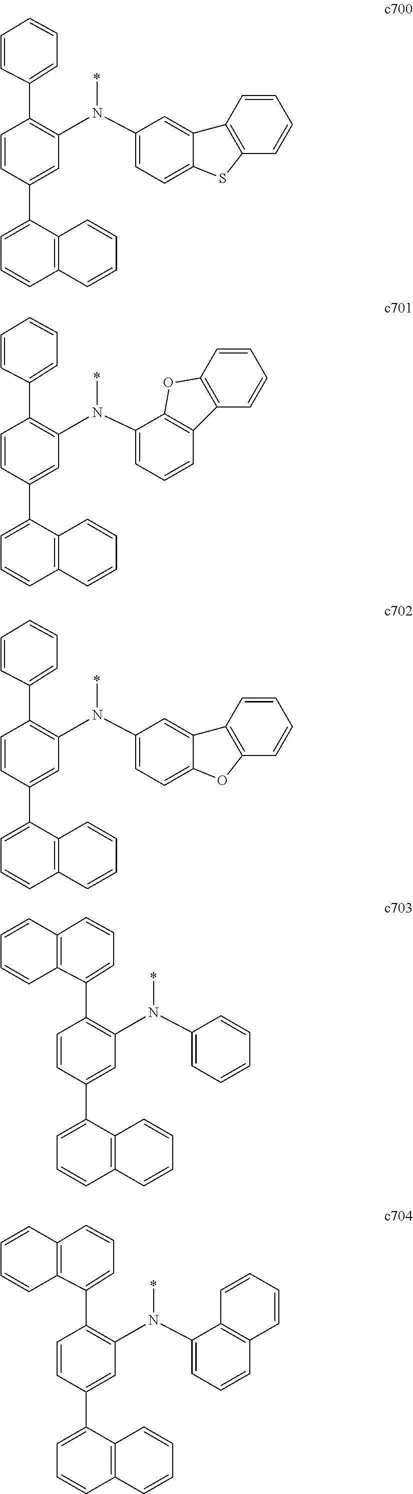

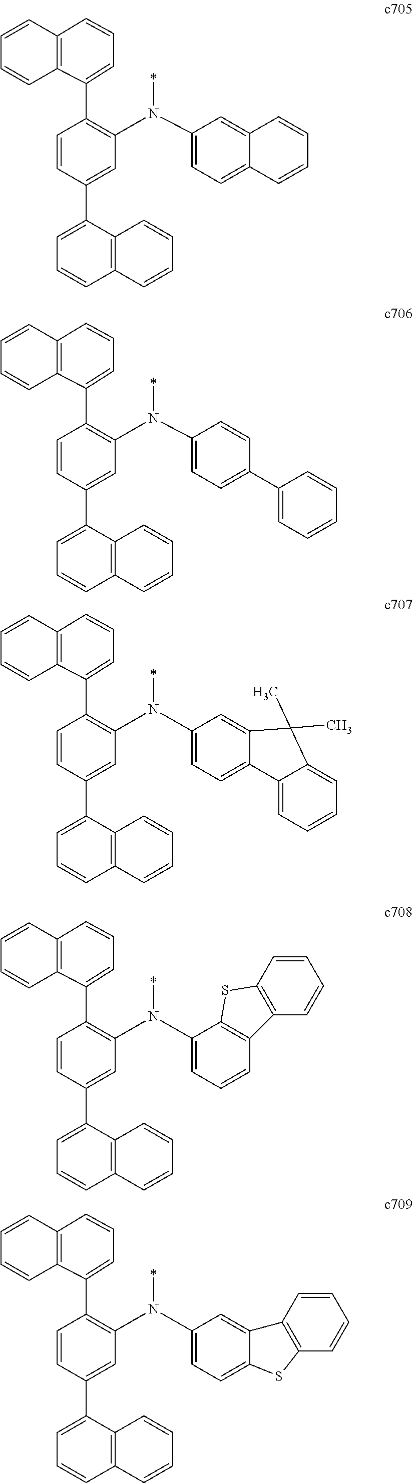

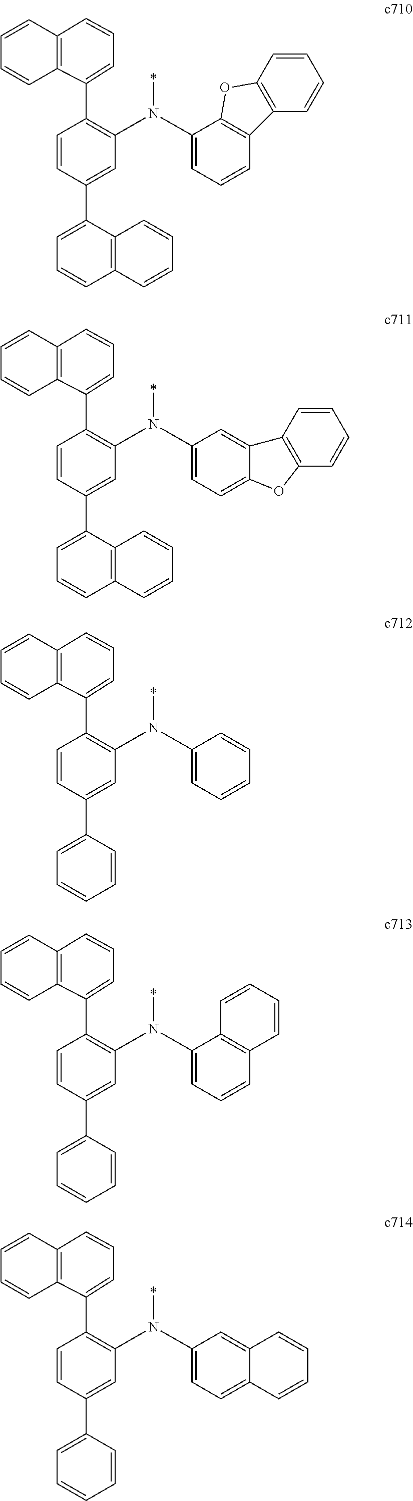

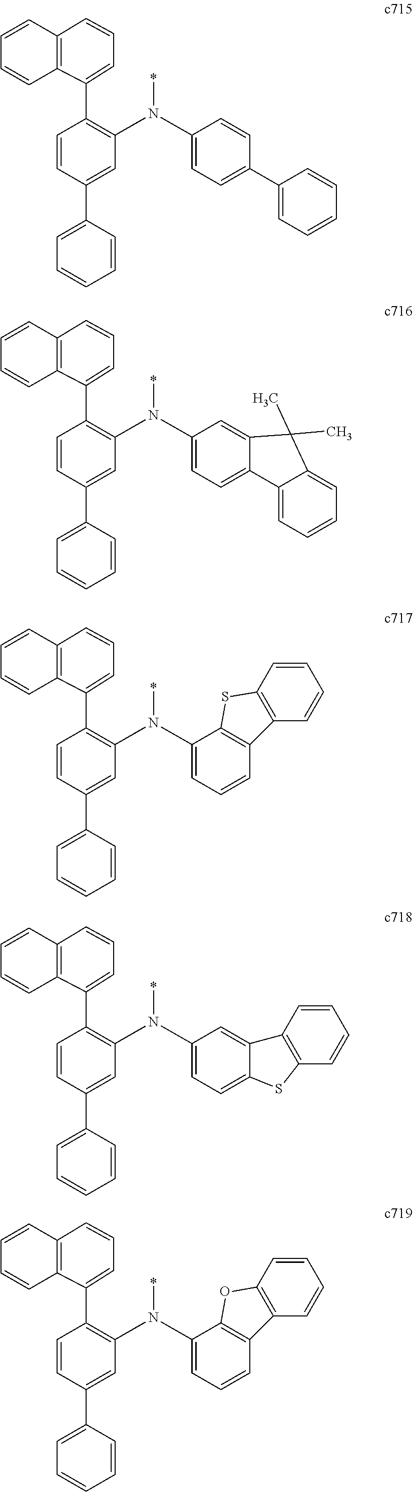

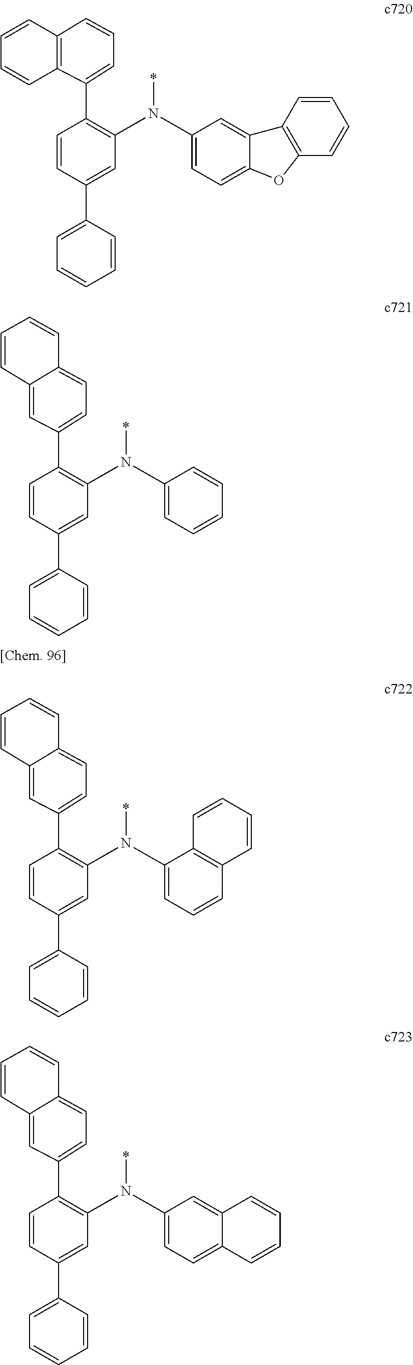

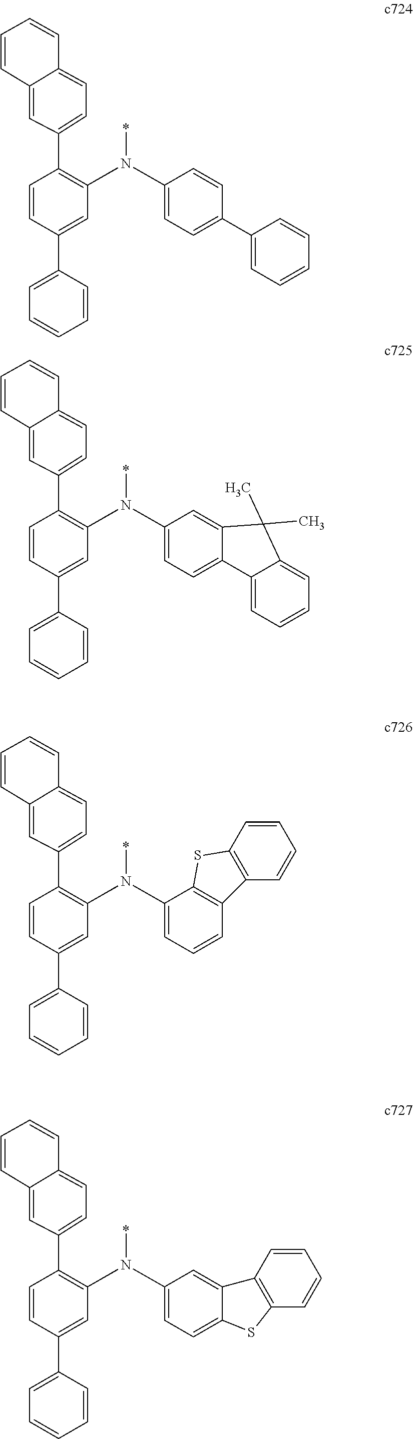

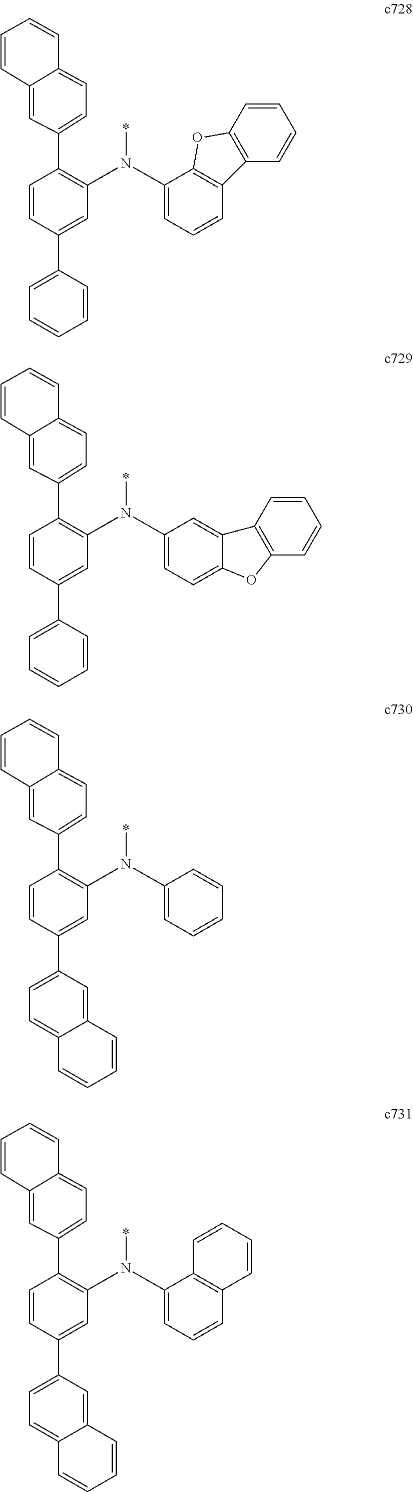

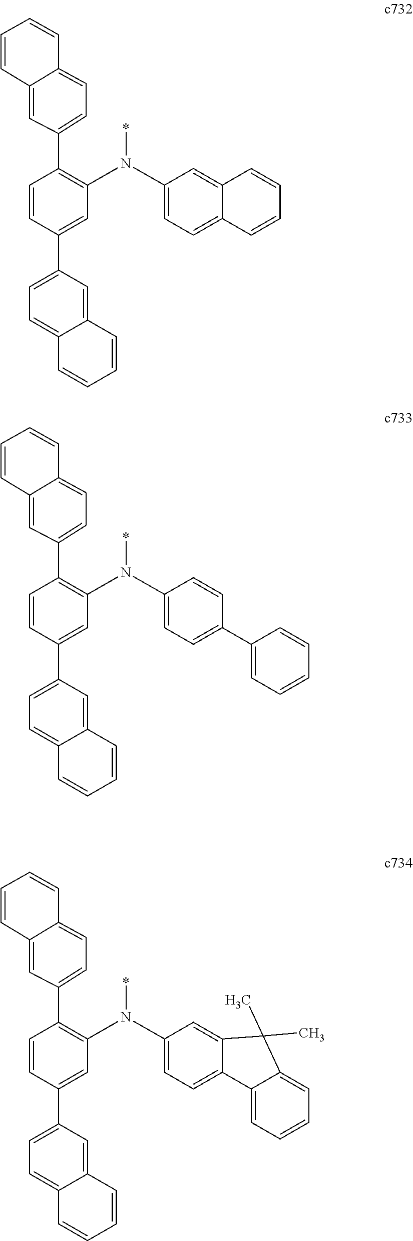

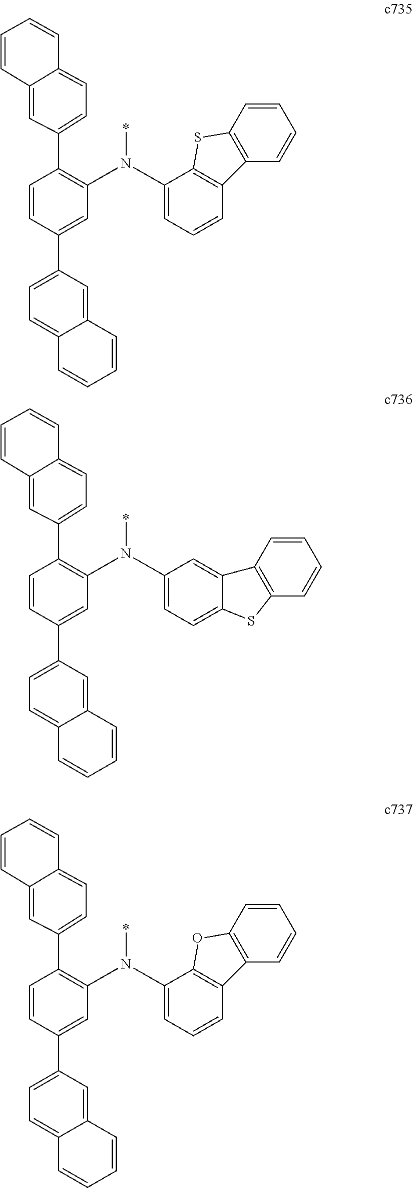

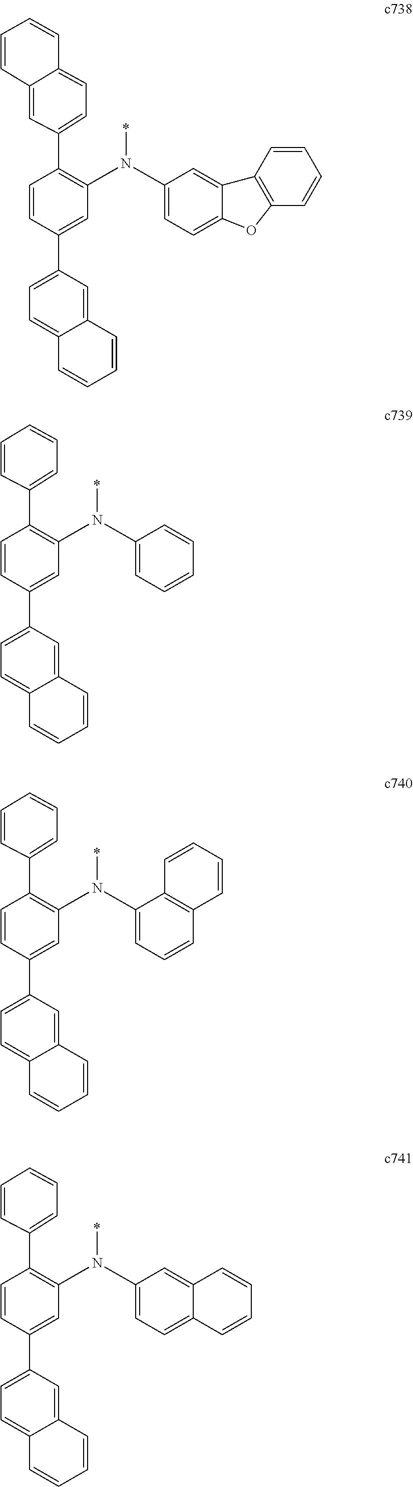

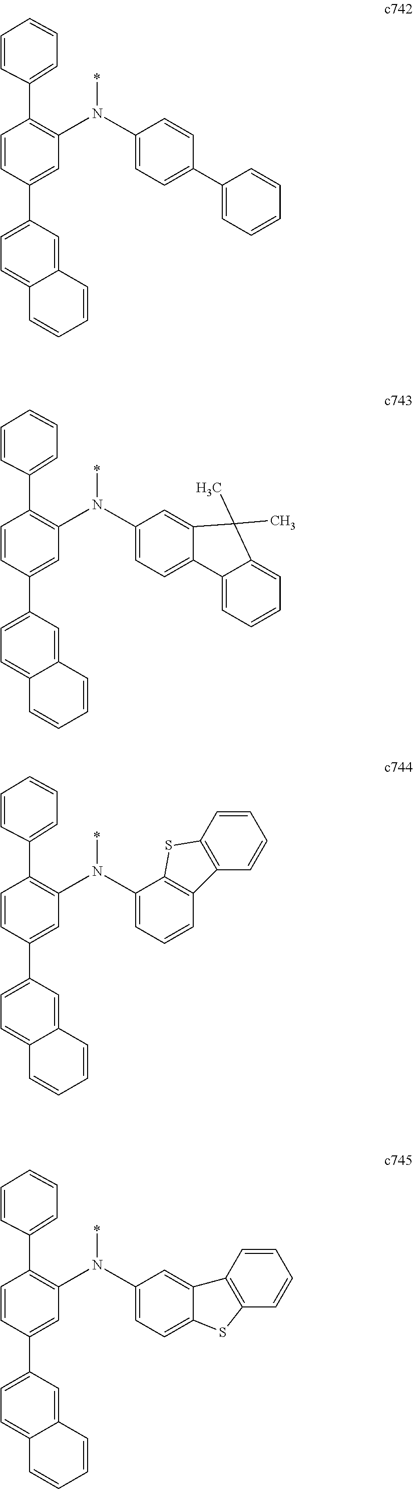

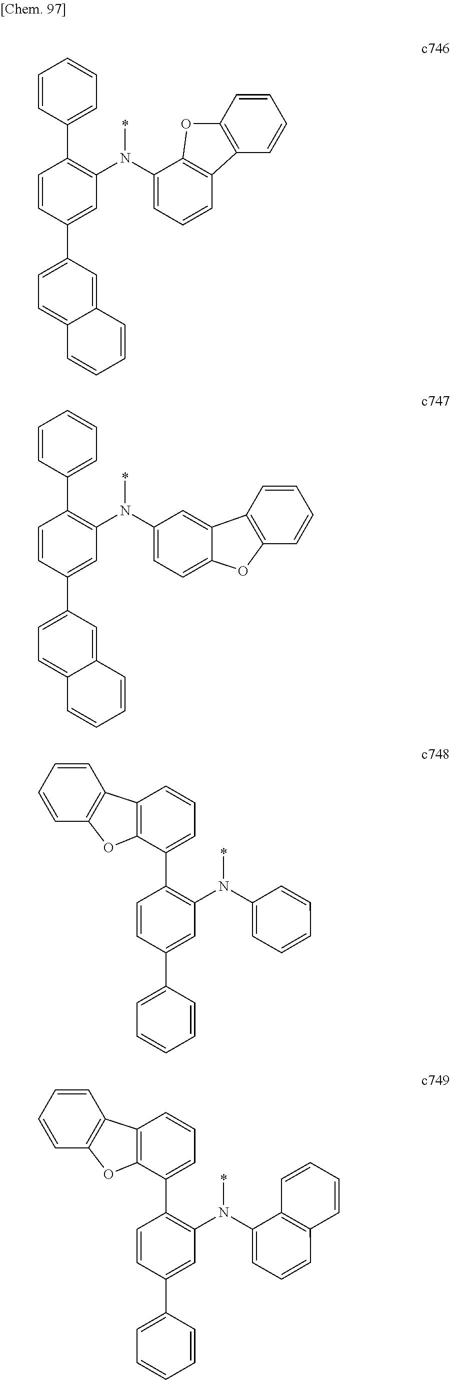

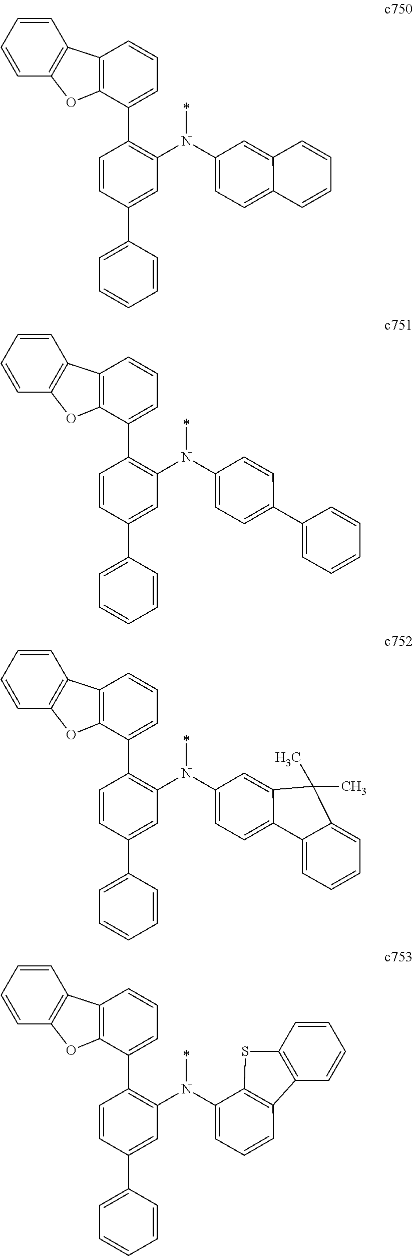 [SMILES: *N(c1ccc2ccccc2c1)c1cc(-c2ccccc2)ccc1-c1cccc2c1oc1ccccc12 ; *N(c1ccc(-c2ccccc2)cc1)c1cc(-c2ccccc2)ccc1-c1cccc2c1oc1ccccc12 ; *N(c1ccc2c(c1)C(C)(C)c1ccccc1-2)c1cc(-c2ccccc2)ccc1-c1cccc2c1oc1ccccc12 ; *N(c1cc(-c2ccccc2)ccc1-c1cccc2c1oc1ccccc12)c1cccc2c1sc1ccccc12]

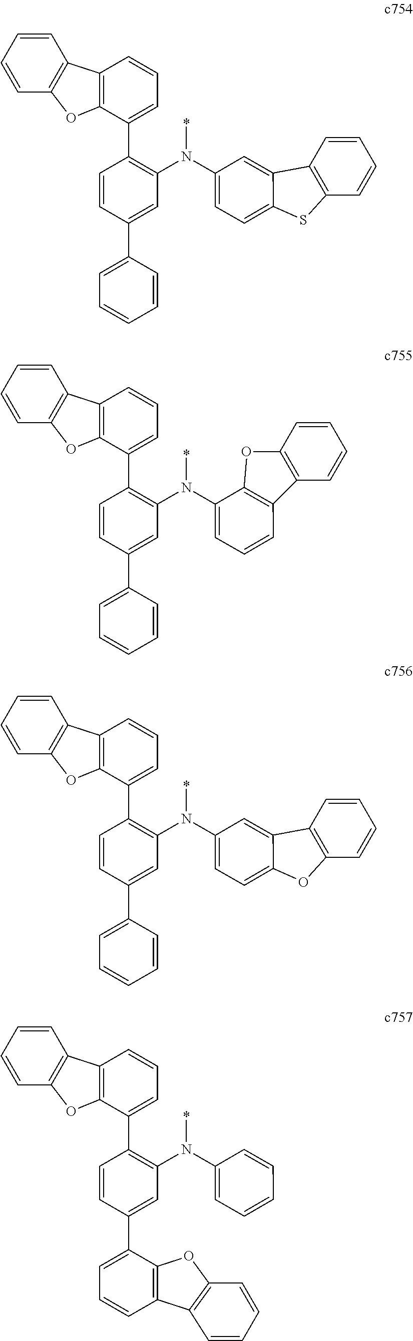 [SMILES: *N(c1ccc2sc3ccccc3c2c1)c1cc(-c2ccccc2)ccc1-c1cccc2c1oc1ccccc12 ; *N(c1cc(-c2ccccc2)ccc1-c1cccc2c1oc1ccccc12)c1cccc2c1oc1ccccc12 ; *N(c1ccc2oc3ccccc3c2c1)c1cc(-c2ccccc2)ccc1-c1cccc2c1oc1ccccc12 ; *N(c1ccccc1)c1cc(-c2cccc3c2oc2ccccc23)ccc1-c1cccc2c1oc1ccccc12]

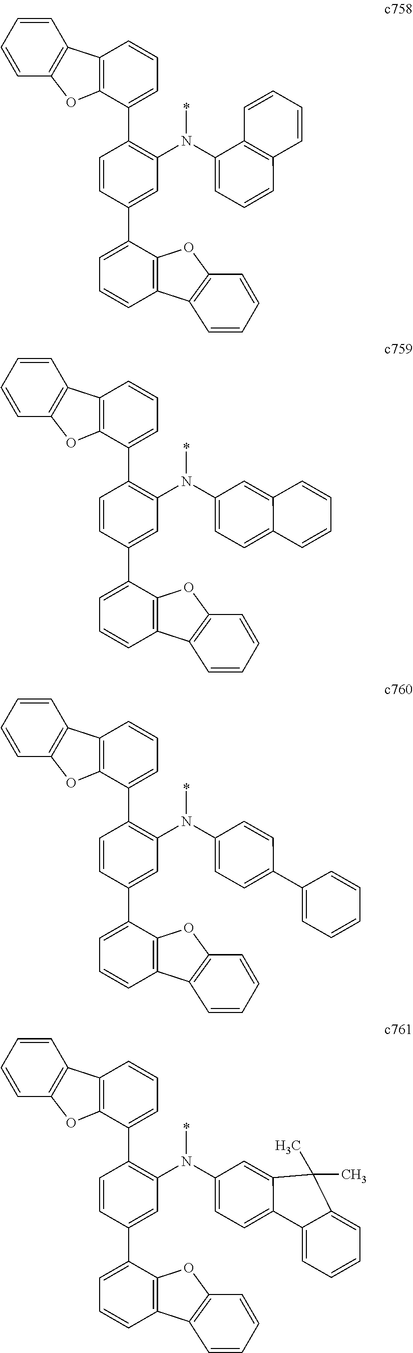 [SMILES: *N(c1cc(-c2cccc3c2oc2ccccc23)ccc1-c1cccc2c1oc1ccccc12)c1cccc2ccccc12 ; *N(c1ccc2ccccc2c1)c1cc(-c2cccc3c2oc2ccccc23)ccc1-c1cccc2c1oc1ccccc12 ; *N(c1ccc(-c2ccccc2)cc1)c1cc(-c2cccc3c2oc2ccccc23)ccc1-c1cccc2c1oc1ccccc12 ; *N(c1ccc2c(c1)C(C)(C)c1ccccc1-2)c1cc(-c2cccc3c2oc2ccccc23)ccc1-c1cccc2c1oc1ccccc12]

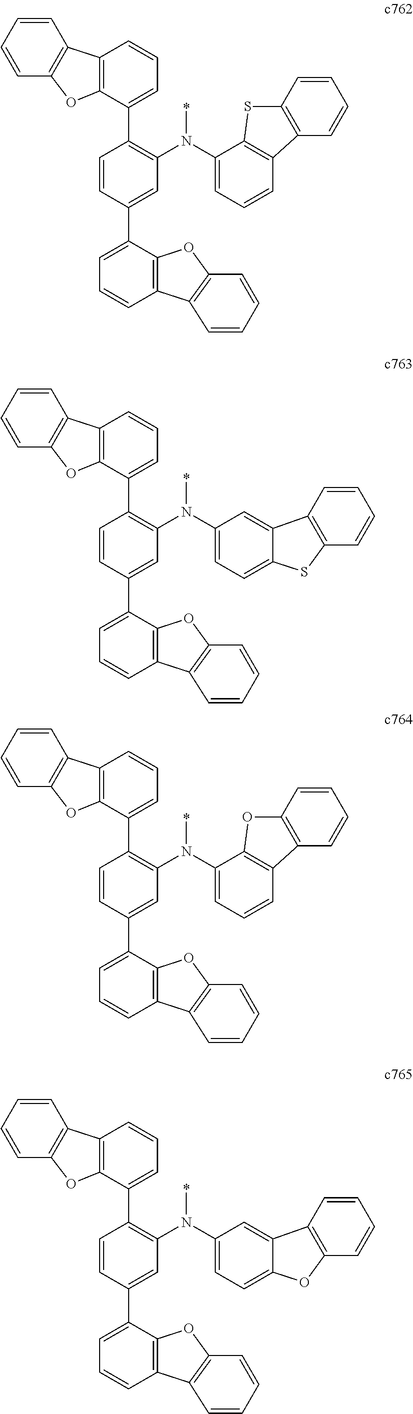 [SMILES: *N(c1cc(-c2cccc3c2oc2ccccc23)ccc1-c1cccc2c1oc1ccccc12)c1cccc2c1sc1ccccc12 ; *N(c1ccc2sc3ccccc3c2c1)c1cc(-c2cccc3c2oc2ccccc23)ccc1-c1cccc2c1oc1ccccc12 ; *N(c1cc(-c2cccc3c2oc2ccccc23)ccc1-c1cccc2c1oc1ccccc12)c1cccc2c1oc1ccccc12 ; *N(c1ccc2oc3ccccc3c2c1)c1cc(-c2cccc3c2oc2ccccc23)ccc1-c1cccc2c1oc1ccccc12]

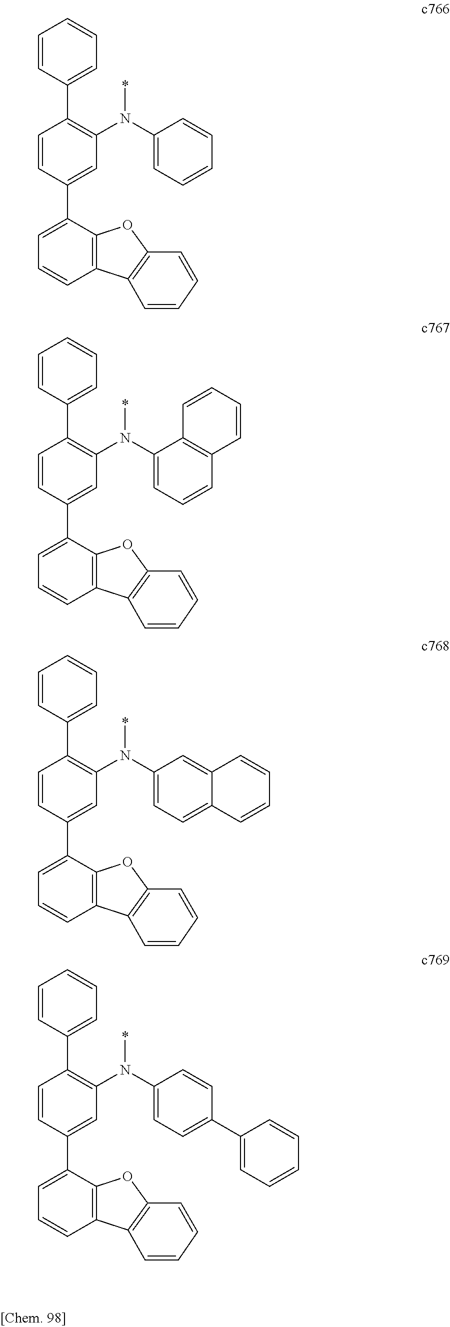 [SMILES: *N(c1ccccc1)c1cc(-c2cccc3c2oc2ccccc23)ccc1-c1ccccc1 ; *N(c1cc(-c2cccc3c2oc2ccccc23)ccc1-c1ccccc1)c1cccc2ccccc12 ; *N(c1ccc2ccccc2c1)c1cc(-c2cccc3c2oc2ccccc23)ccc1-c1ccccc1 ; *N(c1ccc(-c2ccccc2)cc1)c1cc(-c2cccc3c2oc2ccccc23)ccc1-c1ccccc1]

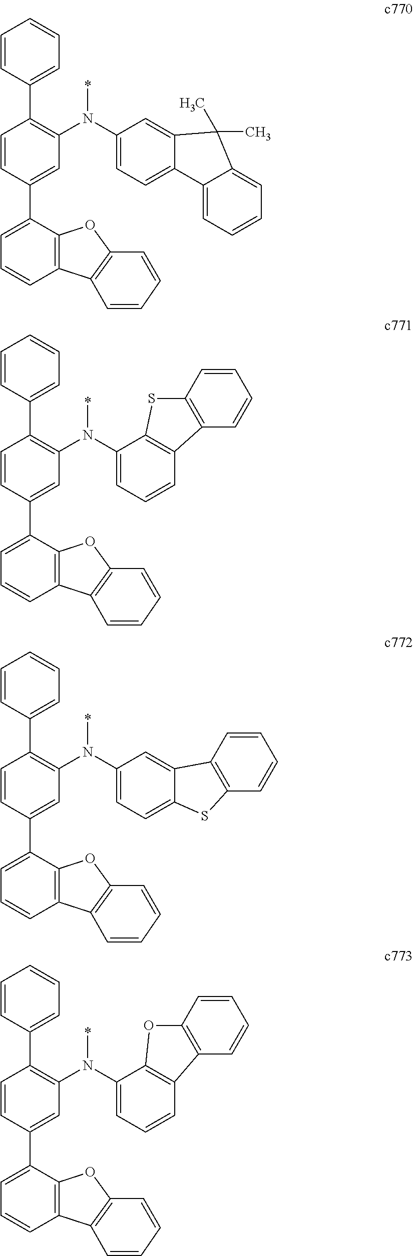 [SMILES: *N(c1ccc2c(c1)C(C)(C)c1ccccc1-2)c1cc(-c2cccc3c2oc2ccccc23)ccc1-c1ccccc1 ; *N(c1cc(-c2cccc3c2oc2ccccc23)ccc1-c1ccccc1)c1cccc2c1sc1ccccc12 ; *N(c1ccc2sc3ccccc3c2c1)c1cc(-c2cccc3c2oc2ccccc23)ccc1-c1ccccc1 ; *N(c1cc(-c2cccc3c2oc2ccccc23)ccc1-c1ccccc1)c1cccc2c1oc1ccccc12]

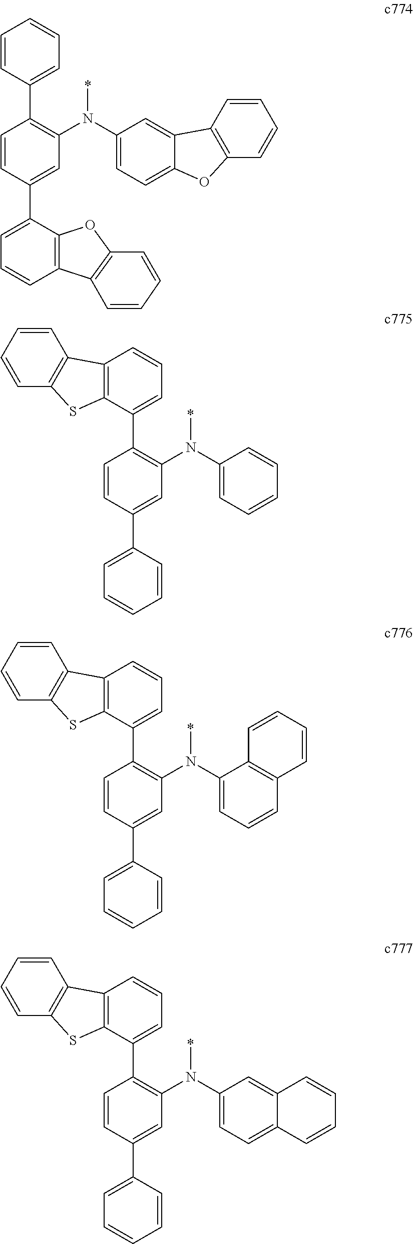

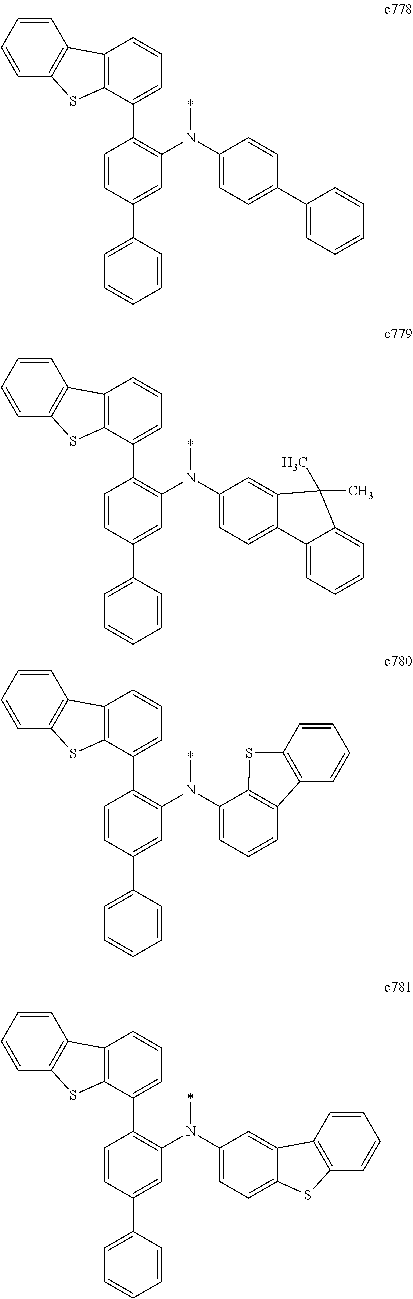

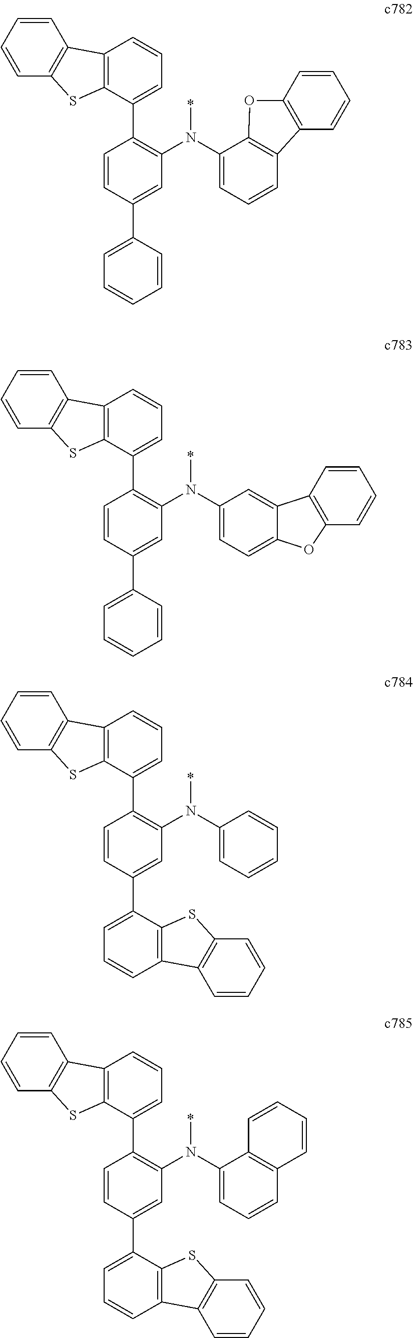 [SMILES: *N(c1cc(-c2ccccc2)ccc1-c1cccc2c1sc1ccccc12)c1cccc2c1oc1ccccc12 ; *N(c1ccc2oc3ccccc3c2c1)c1cc(-c2ccccc2)ccc1-c1cccc2c1sc1ccccc12 ; *N(c1ccccc1)c1cc(-c2cccc3c2sc2ccccc23)ccc1-c1cccc2c1sc1ccccc12 ; *N(c1cc(-c2cccc3c2sc2ccccc23)ccc1-c1cccc2c1sc1ccccc12)c1cccc2ccccc12]

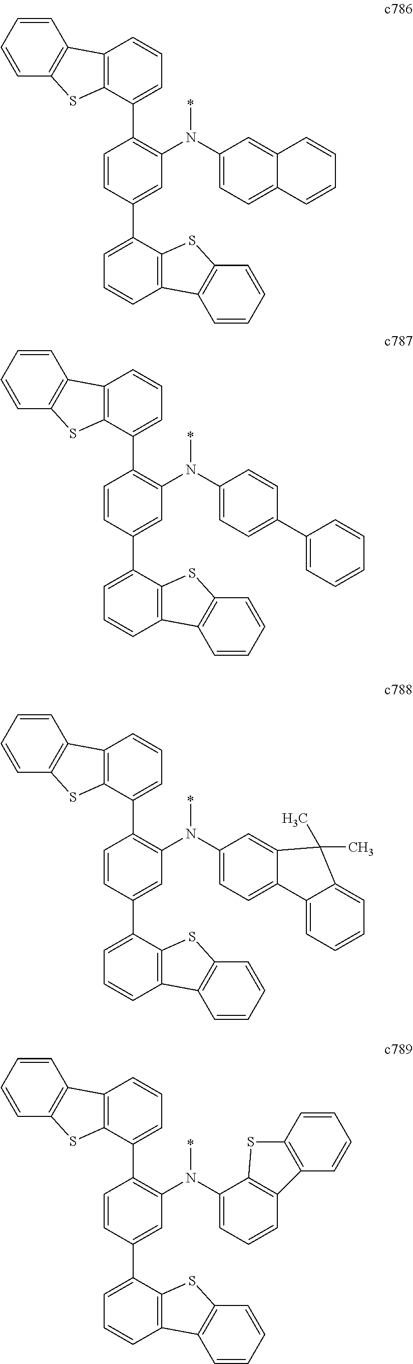 [SMILES: *N(c1ccc2ccccc2c1)c1cc(-c2cccc3c2sc2ccccc23)ccc1-c1cccc2c1sc1ccccc12 ; *N(c1ccc(-c2ccccc2)cc1)c1cc(-c2cccc3c2sc2ccccc23)ccc1-c1cccc2c1sc1ccccc12 ; *N(c1ccc2c(c1)C(C)(C)c1ccccc1-2)c1cc(-c2cccc3c2sc2ccccc23)ccc1-c1cccc2c1sc1ccccc12 ; *N(c1cc(-c2cccc3c2sc2ccccc23)ccc1-c1cccc2c1sc1ccccc12)c1cccc2c1sc1ccccc12]

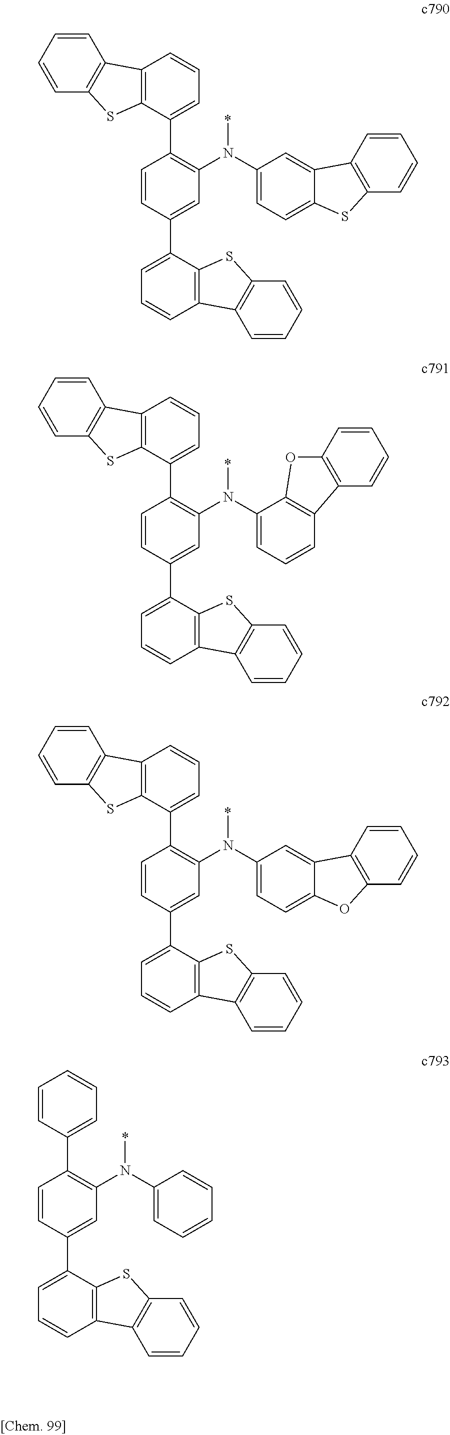 [SMILES: *N(c1ccc2sc3ccccc3c2c1)c1cc(-c2cccc3c2sc2ccccc23)ccc1-c1cccc2c1sc1ccccc12 ; *N(c1cc(-c2cccc3c2sc2ccccc23)ccc1-c1cccc2c1sc1ccccc12)c1cccc2c1oc1ccccc12 ; *N(c1ccc2oc3ccccc3c2c1)c1cc(-c2cccc3c2sc2ccccc23)ccc1-c1cccc2c1sc1ccccc12 ; *N(c1ccccc1)c1cc(-c2cccc3c2sc2ccccc23)ccc1-c1ccccc1]

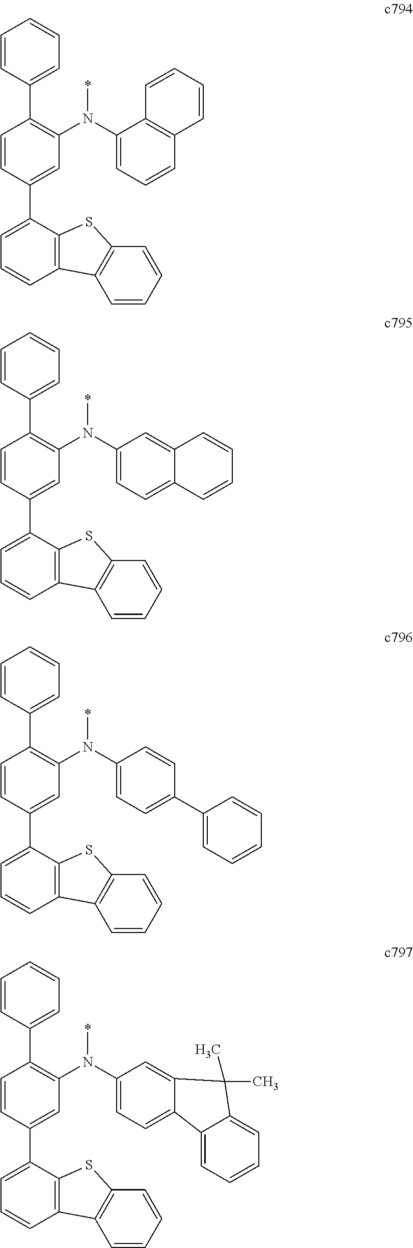 [SMILES: *N(c1cc(-c2cccc3c2sc2ccccc23)ccc1-c1ccccc1)c1cccc2ccccc12 ; *N(c1ccc2ccccc2c1)c1cc(-c2cccc3c2sc2ccccc23)ccc1-c1ccccc1 ; *N(c1ccc(-c2ccccc2)cc1)c1cc(-c2cccc3c2sc2ccccc23)ccc1-c1ccccc1 ; *N(c1ccc2c(c1)C(C)(C)c1ccccc1-2)c1cc(-c2cccc3c2sc2ccccc23)ccc1-c1ccccc1]

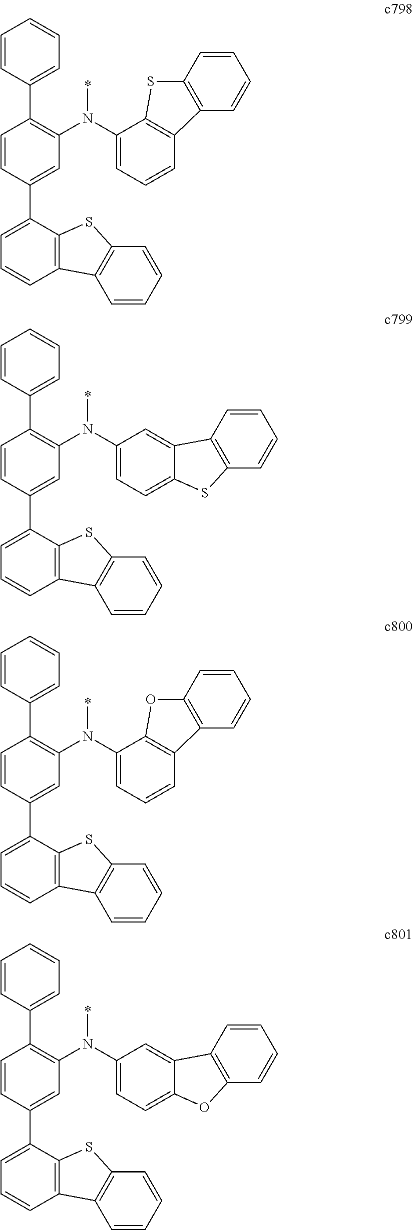

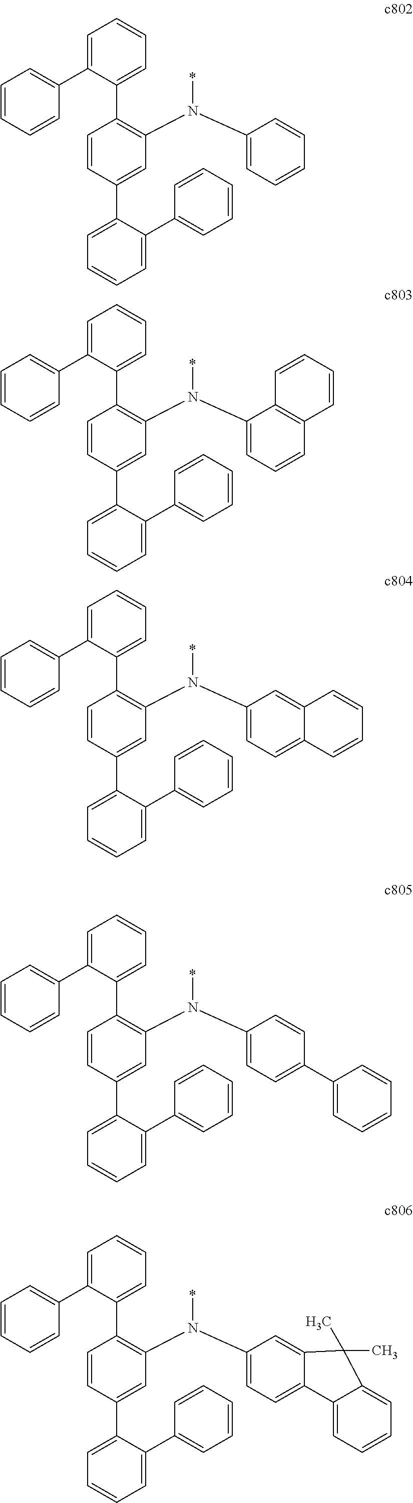

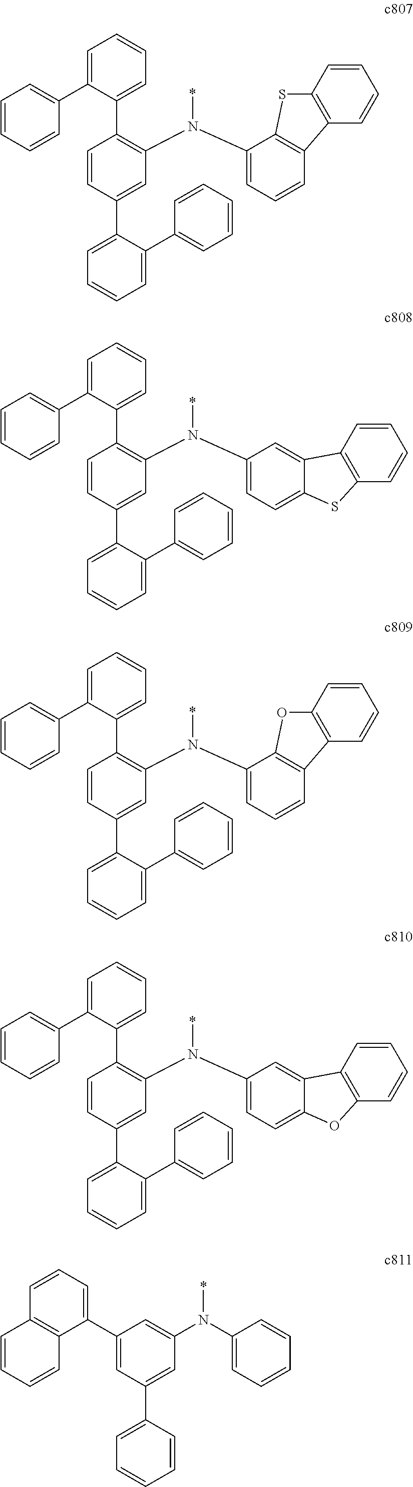

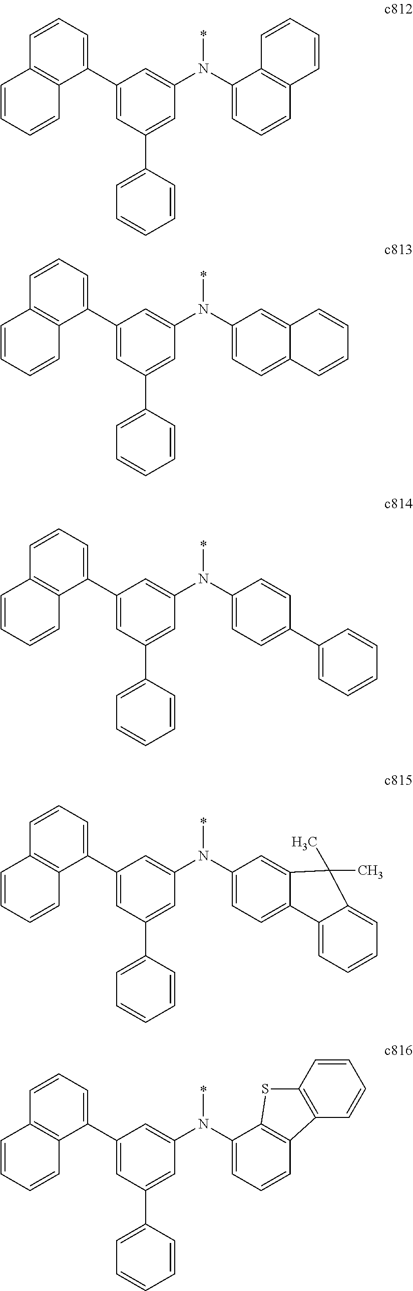 [SMILES: *N(c1cc(-c2ccccc2)cc(-c2cccc3ccccc23)c1)c1cccc2ccccc12 ; *N(c1cc(-c2ccccc2)cc(-c2cccc3ccccc23)c1)c1ccc2ccccc2c1 ; *N(c1ccc(-c2ccccc2)cc1)c1cc(-c2ccccc2)cc(-c2cccc3ccccc23)c1 ; *N(c1cc(-c2ccccc2)cc(-c2cccc3ccccc23)c1)c1ccc2c(c1)C(C)(C)c1ccccc1-2 ; *N(c1cc(-c2ccccc2)cc(-c2cccc3ccccc23)c1)c1cccc2c1sc1ccccc12]

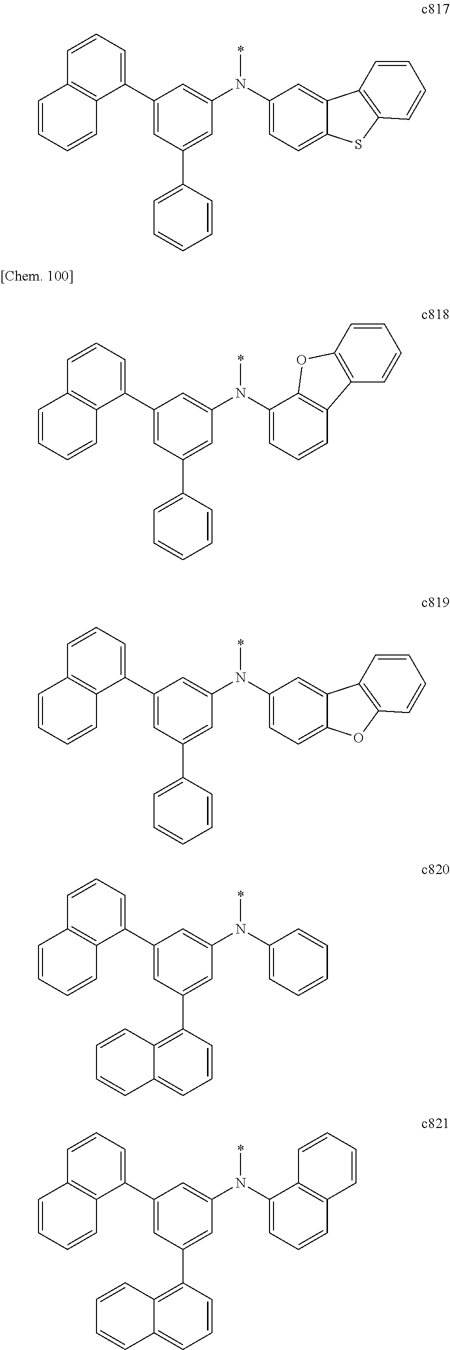 [SMILES: *N(c1cc(-c2ccccc2)cc(-c2cccc3ccccc23)c1)c1ccc2sc3ccccc3c2c1 ; *N(c1cc(-c2ccccc2)cc(-c2cccc3ccccc23)c1)c1cccc2c1oc1ccccc12 ; *N(c1cc(-c2ccccc2)cc(-c2cccc3ccccc23)c1)c1ccc2oc3ccccc3c2c1 ; *N(c1ccccc1)c1cc(-c2cccc3ccccc23)cc(-c2cccc3ccccc23)c1 ; *N(c1cc(-c2cccc3ccccc23)cc(-c2cccc3ccccc23)c1)c1cccc2ccccc12]

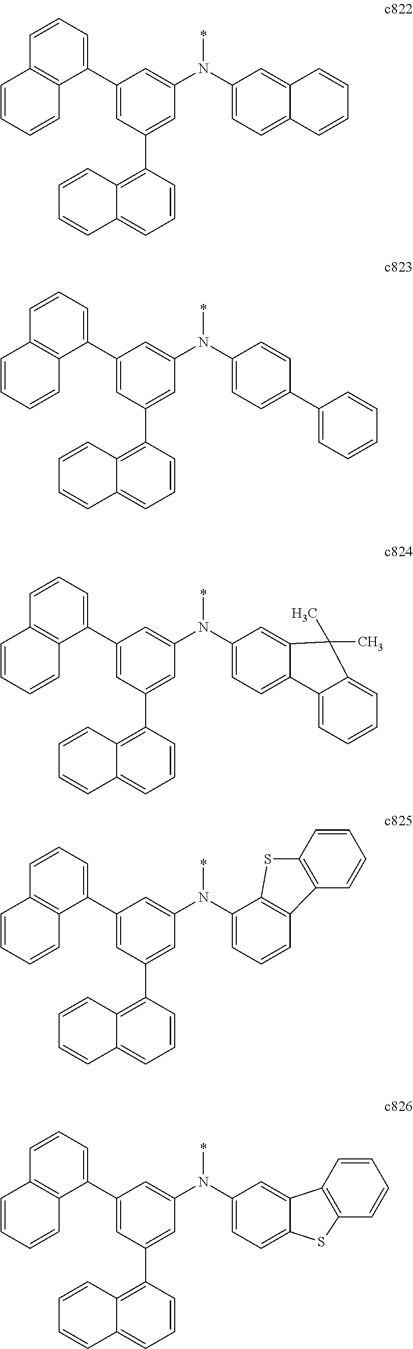 [SMILES: *N(c1cc(-c2cccc3ccccc23)cc(-c2cccc3ccccc23)c1)c1ccc2ccccc2c1 ; *N(c1ccc(-c2ccccc2)cc1)c1cc(-c2cccc3ccccc23)cc(-c2cccc3ccccc23)c1 ; *N(c1cc(-c2cccc3ccccc23)cc(-c2cccc3ccccc23)c1)c1ccc2c(c1)C(C)(C)c1ccccc1-2 ; *N(c1cc(-c2cccc3ccccc23)cc(-c2cccc3ccccc23)c1)c1cccc2c1sc1ccccc12 ; *N(c1cc(-c2cccc3ccccc23)cc(-c2cccc3ccccc23)c1)c1ccc2sc3ccccc3c2c1]

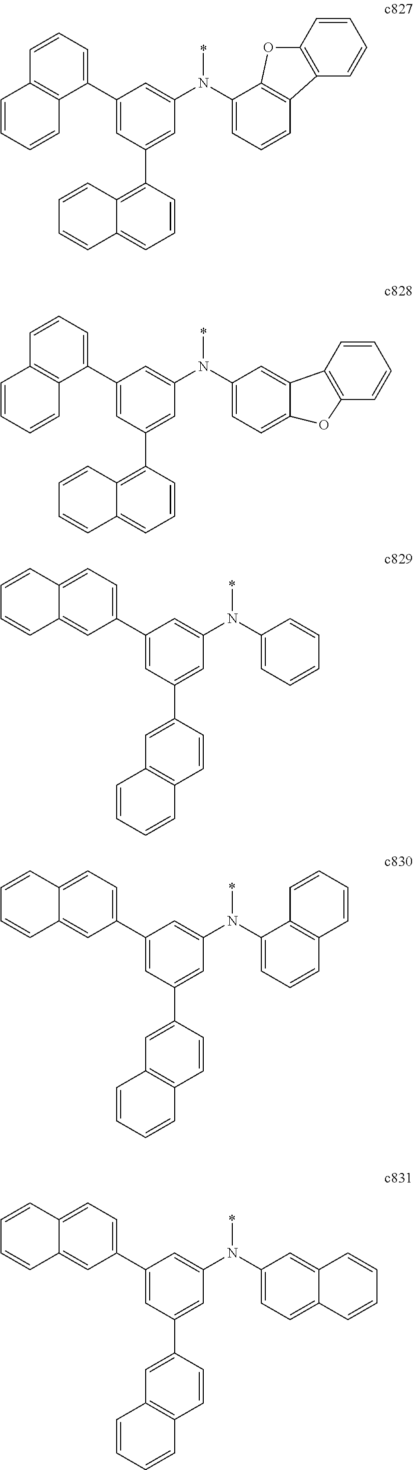

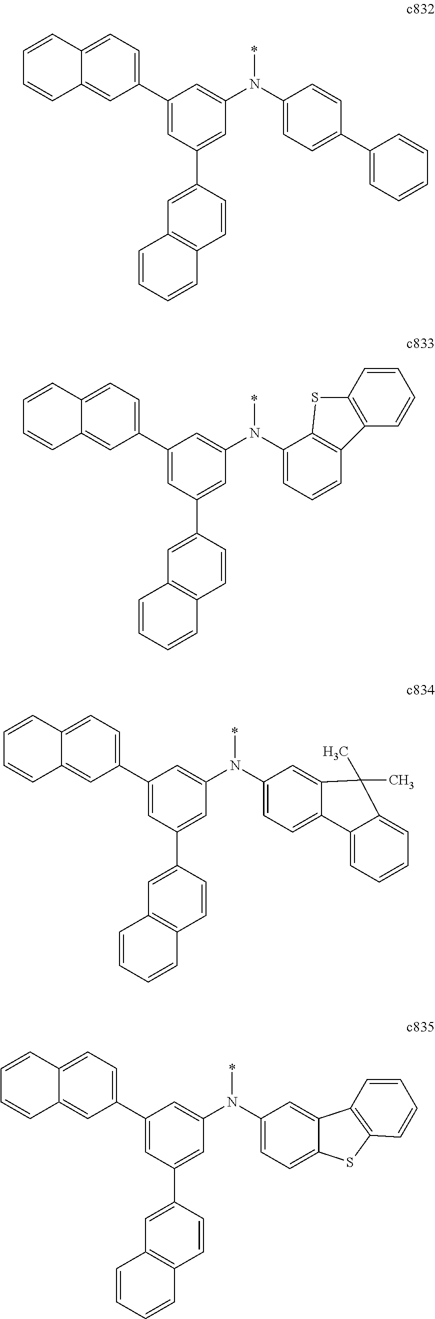 [SMILES: *N(c1ccc(-c2ccccc2)cc1)c1cc(-c2ccc3ccccc3c2)cc(-c2ccc3ccccc3c2)c1 ; *N(c1cc(-c2ccc3ccccc3c2)cc(-c2ccc3ccccc3c2)c1)c1cccc2c1sc1ccccc12 ; *N(c1cc(-c2ccc3ccccc3c2)cc(-c2ccc3ccccc3c2)c1)c1ccc2c(c1)C(C)(C)c1ccccc1-2 ; *N(c1cc(-c2ccc3ccccc3c2)cc(-c2ccc3ccccc3c2)c1)c1ccc2sc3ccccc3c2c1]

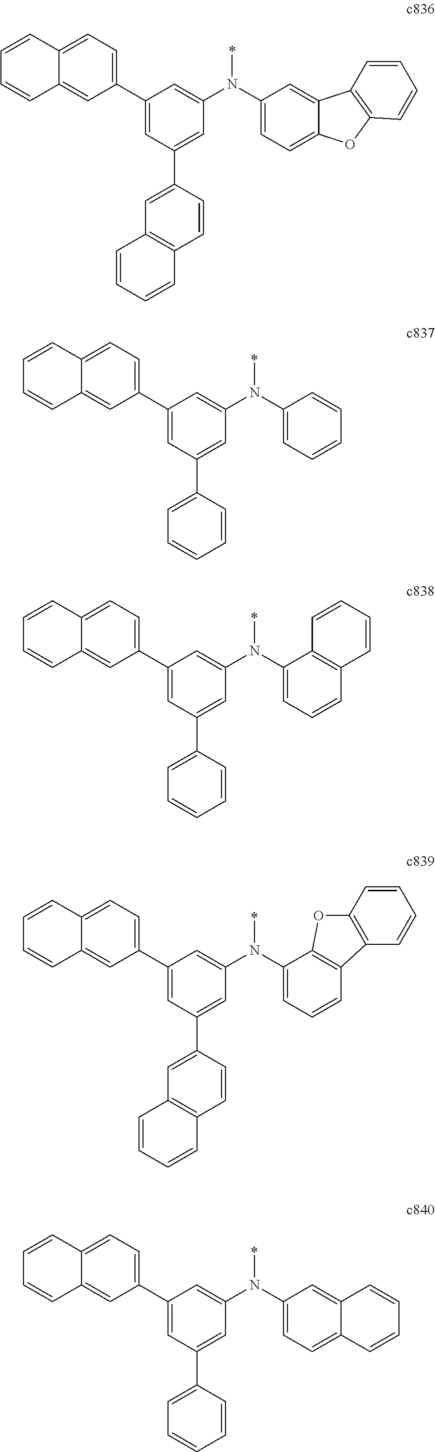 [SMILES: *N(c1cc(-c2ccc3ccccc3c2)cc(-c2ccc3ccccc3c2)c1)c1ccc2oc3ccccc3c2c1 ; *N(c1ccccc1)c1cc(-c2ccccc2)cc(-c2ccc3ccccc3c2)c1 ; *N(c1cc(-c2ccccc2)cc(-c2ccc3ccccc3c2)c1)c1cccc2ccccc12 ; *N(c1cc(-c2ccc3ccccc3c2)cc(-c2ccc3ccccc3c2)c1)c1cccc2c1oc1ccccc12 ; *N(c1cc(-c2ccccc2)cc(-c2ccc3ccccc3c2)c1)c1ccc2ccccc2c1]

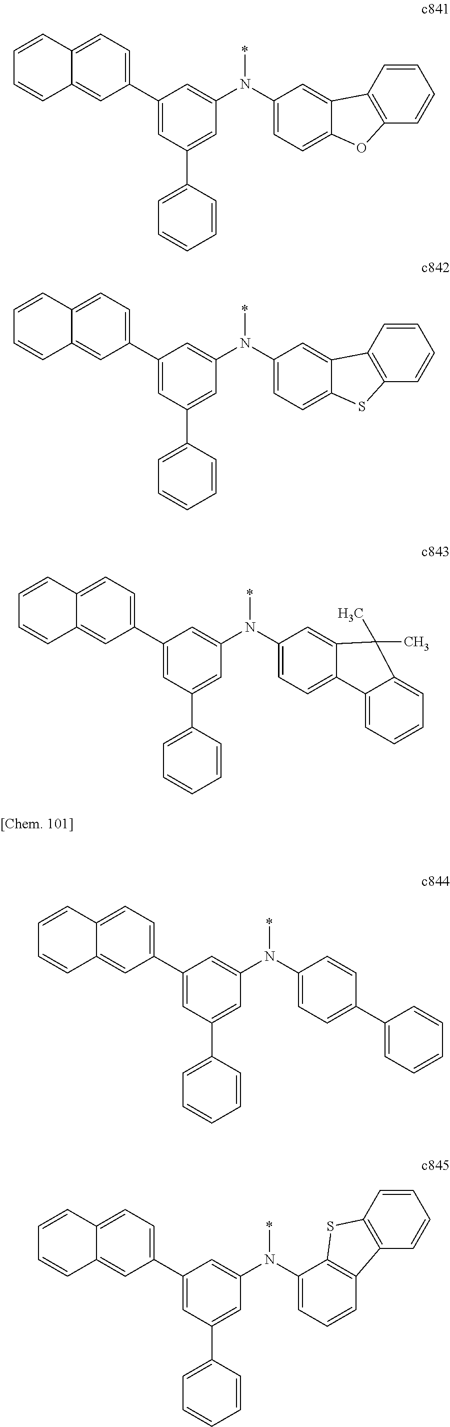 [SMILES: *N(c1cc(-c2ccccc2)cc(-c2ccc3ccccc3c2)c1)c1ccc2oc3ccccc3c2c1 ; *N(c1cc(-c2ccccc2)cc(-c2ccc3ccccc3c2)c1)c1ccc2sc3ccccc3c2c1 ; *N(c1cc(-c2ccccc2)cc(-c2ccc3ccccc3c2)c1)c1ccc2c(c1)C(C)(C)c1ccccc1-2 ; *N(c1ccc(-c2ccccc2)cc1)c1cc(-c2ccccc2)cc(-c2ccc3ccccc3c2)c1 ; *N(c1cc(-c2ccccc2)cc(-c2ccc3ccccc3c2)c1)c1cccc2c1sc1ccccc12]

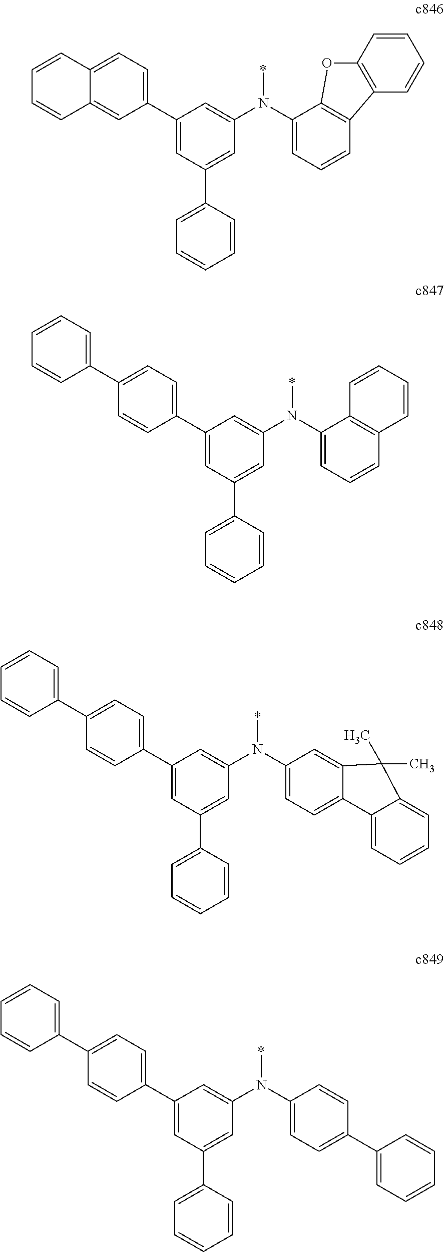 [SMILES: *N(c1cc(-c2ccccc2)cc(-c2ccc3ccccc3c2)c1)c1cccc2c1oc1ccccc12 ; *N(c1cc(-c2ccccc2)cc(-c2ccc(-c3ccccc3)cc2)c1)c1cccc2ccccc12 ; *N(c1cc(-c2ccccc2)cc(-c2ccc(-c3ccccc3)cc2)c1)c1ccc2c(c1)C(C)(C)c1ccccc1-2 ; *N(c1ccc(-c2ccccc2)cc1)c1cc(-c2ccccc2)cc(-c2ccc(-c3ccccc3)cc2)c1]

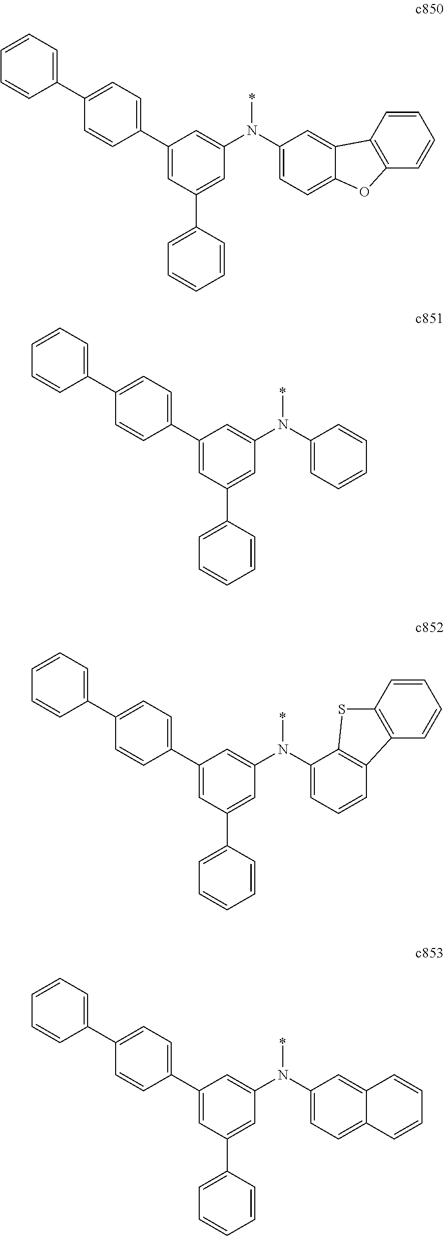 [SMILES: *N(c1cc(-c2ccccc2)cc(-c2ccc(-c3ccccc3)cc2)c1)c1ccc2oc3ccccc3c2c1 ; *N(c1ccccc1)c1cc(-c2ccccc2)cc(-c2ccc(-c3ccccc3)cc2)c1 ; *N(c1cc(-c2ccccc2)cc(-c2ccc(-c3ccccc3)cc2)c1)c1cccc2c1sc1ccccc12 ; *N(c1cc(-c2ccccc2)cc(-c2ccc(-c3ccccc3)cc2)c1)c1ccc2ccccc2c1]

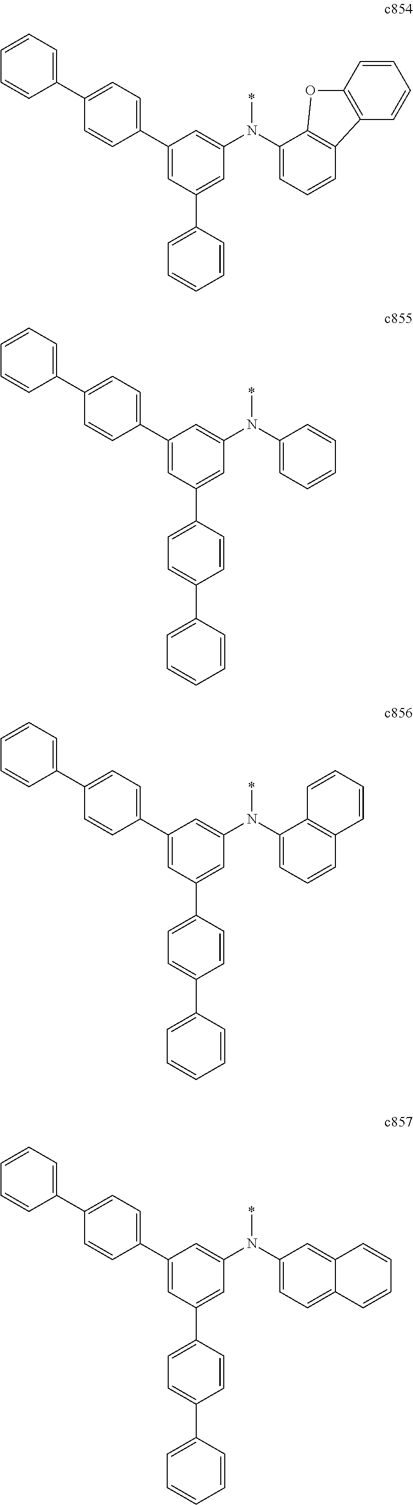 [SMILES: *N(c1cc(-c2ccccc2)cc(-c2ccc(-c3ccccc3)cc2)c1)c1cccc2c1oc1ccccc12 ; *N(c1ccccc1)c1cc(-c2ccc(-c3ccccc3)cc2)cc(-c2ccc(-c3ccccc3)cc2)c1 ; *N(c1cc(-c2ccc(-c3ccccc3)cc2)cc(-c2ccc(-c3ccccc3)cc2)c1)c1cccc2ccccc12 ; *N(c1cc(-c2ccc(-c3ccccc3)cc2)cc(-c2ccc(-c3ccccc3)cc2)c1)c1ccc2ccccc2c1]

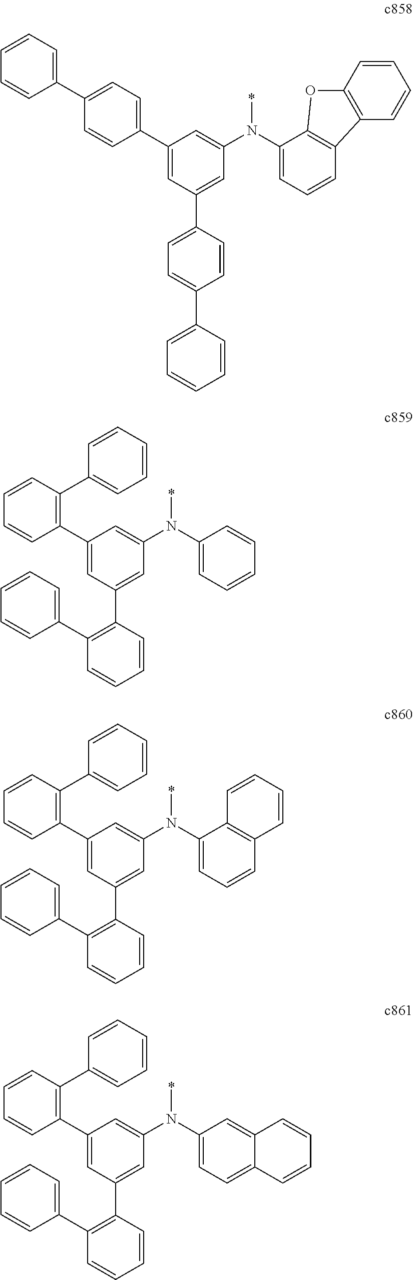

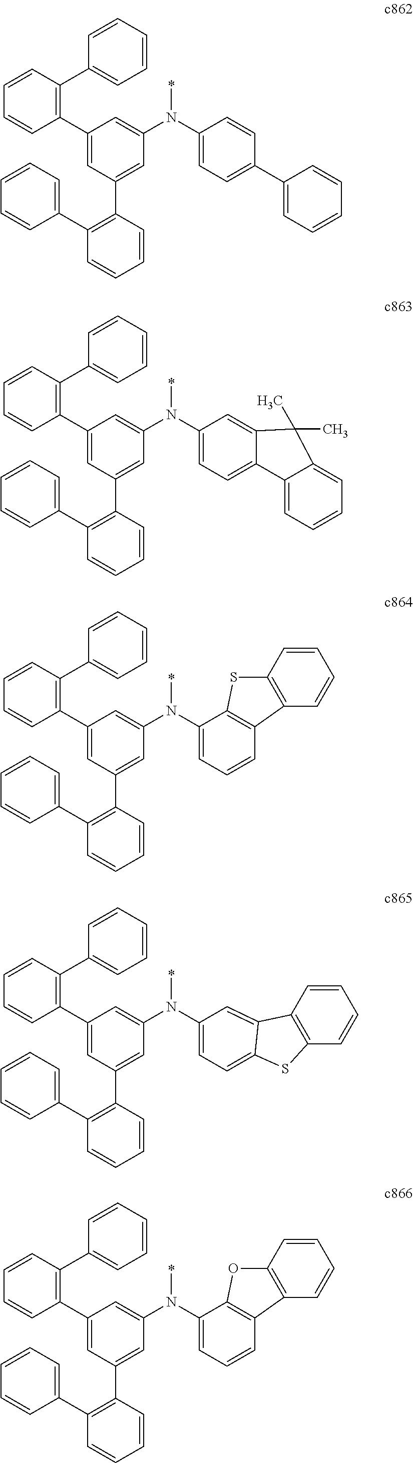

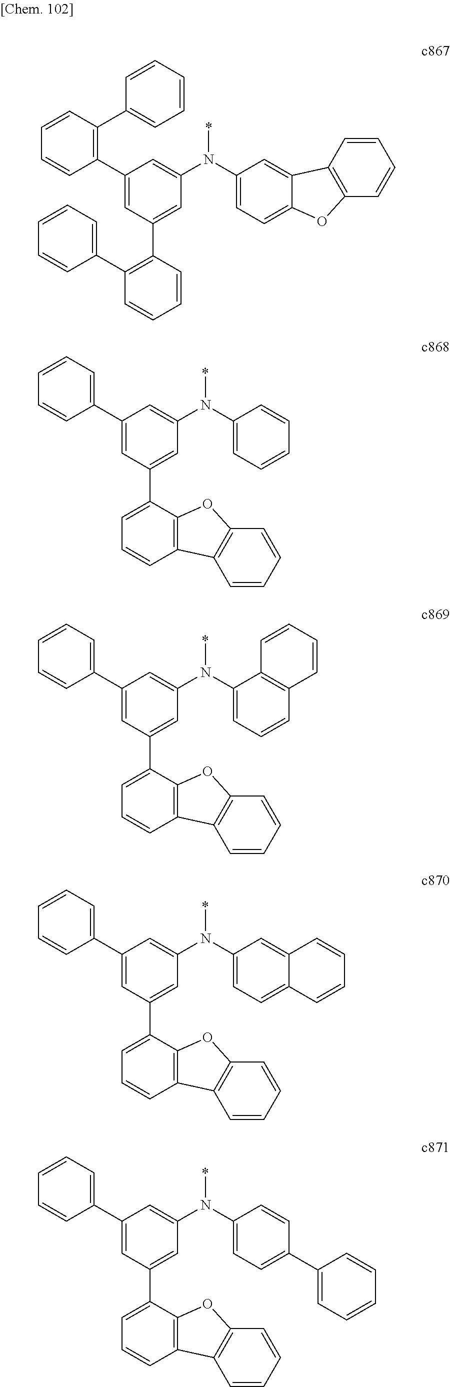 [SMILES: *N(c1cc(-c2ccccc2-c2ccccc2)cc(-c2ccccc2-c2ccccc2)c1)c1ccc2oc3ccccc3c2c1 ; *N(c1ccccc1)c1cc(-c2ccccc2)cc(-c2cccc3c2oc2ccccc23)c1 ; *N(c1cc(-c2ccccc2)cc(-c2cccc3c2oc2ccccc23)c1)c1cccc2ccccc12 ; *N(c1cc(-c2ccccc2)cc(-c2cccc3c2oc2ccccc23)c1)c1ccc2ccccc2c1 ; *N(c1ccc(-c2ccccc2)cc1)c1cc(-c2ccccc2)cc(-c2cccc3c2oc2ccccc23)c1]

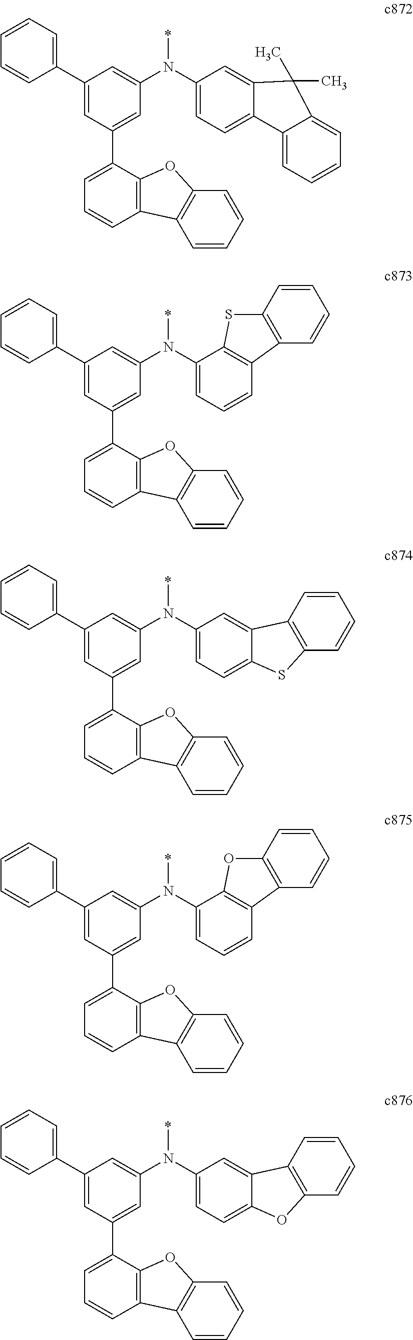 [SMILES: *N(c1cc(-c2ccccc2)cc(-c2cccc3c2oc2ccccc23)c1)c1ccc2c(c1)C(C)(C)c1ccccc1-2 ; *N(c1cc(-c2ccccc2)cc(-c2cccc3c2oc2ccccc23)c1)c1cccc2c1sc1ccccc12 ; *N(c1cc(-c2ccccc2)cc(-c2cccc3c2oc2ccccc23)c1)c1ccc2sc3ccccc3c2c1 ; *N(c1cc(-c2ccccc2)cc(-c2cccc3c2oc2ccccc23)c1)c1cccc2c1oc1ccccc12 ; *N(c1cc(-c2ccccc2)cc(-c2cccc3c2oc2ccccc23)c1)c1ccc2oc3ccccc3c2c1]

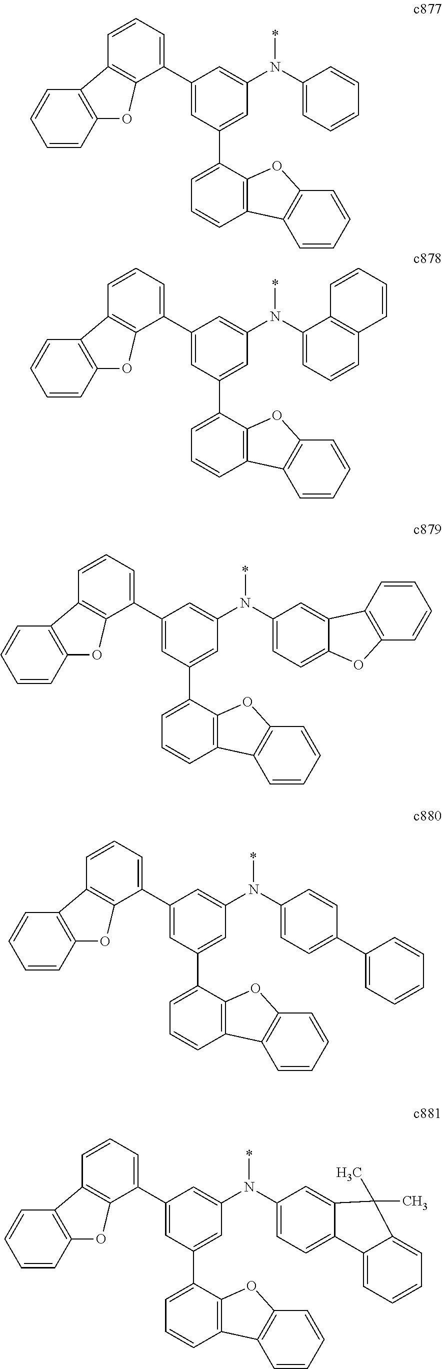

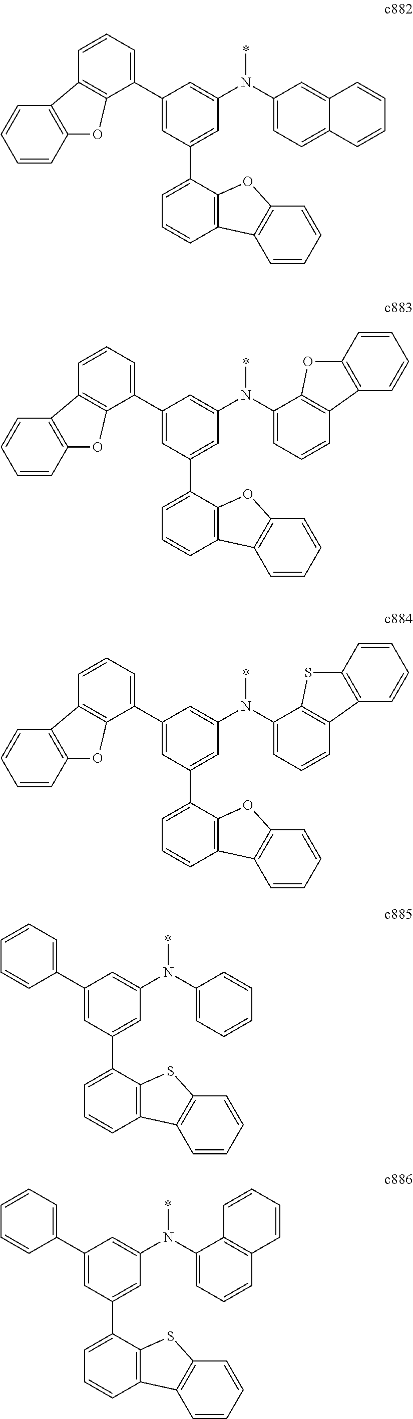

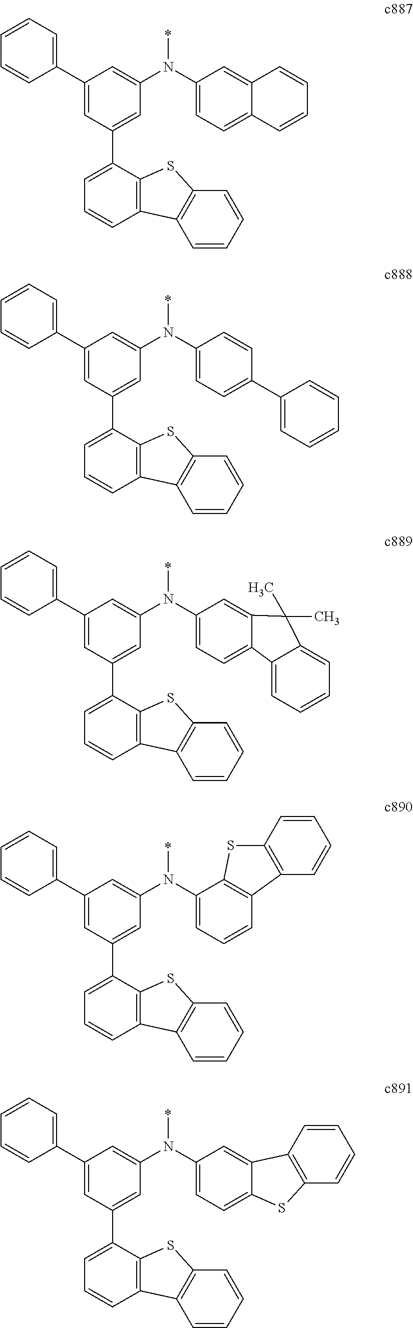 [SMILES: *N(c1cc(-c2ccccc2)cc(-c2cccc3c2sc2ccccc23)c1)c1ccc2ccccc2c1 ; *N(c1ccc(-c2ccccc2)cc1)c1cc(-c2ccccc2)cc(-c2cccc3c2sc2ccccc23)c1 ; *N(c1cc(-c2ccccc2)cc(-c2cccc3c2sc2ccccc23)c1)c1ccc2c(c1)C(C)(C)c1ccccc1-2 ; *N(c1cc(-c2ccccc2)cc(-c2cccc3c2sc2ccccc23)c1)c1cccc2c1sc1ccccc12 ; *N(c1cc(-c2ccccc2)cc(-c2cccc3c2sc2ccccc23)c1)c1ccc2sc3ccccc3c2c1]

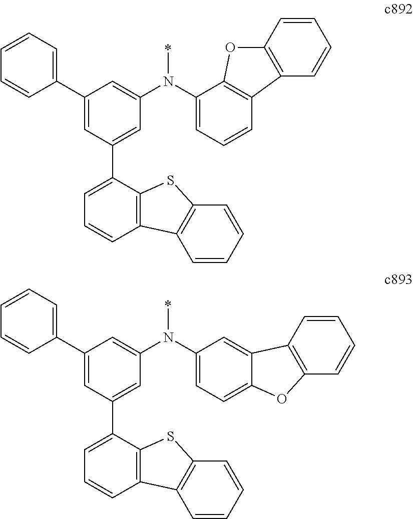 [SMILES: *N(c1cc(-c2ccccc2)cc(-c2cccc3c2sc2ccccc23)c1)c1cccc2c1oc1ccccc12 ; *N(c1cc(-c2ccccc2)cc(-c2cccc3c2sc2ccccc23)c1)c1ccc2oc3ccccc3c2c1]

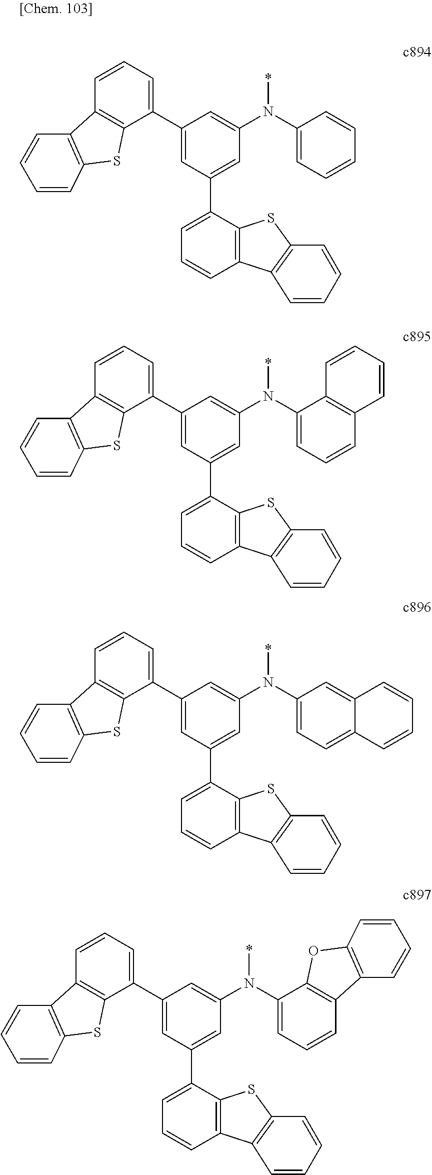 [SMILES: *N(c1ccccc1)c1cc(-c2cccc3c2sc2ccccc23)cc(-c2cccc3c2sc2ccccc23)c1 ; *N(c1cc(-c2cccc3c2sc2ccccc23)cc(-c2cccc3c2sc2ccccc23)c1)c1cccc2ccccc12 ; *N(c1cc(-c2cccc3c2sc2ccccc23)cc(-c2cccc3c2sc2ccccc23)c1)c1ccc2ccccc2c1 ; *N(c1cc(-c2cccc3c2sc2ccccc23)cc(-c2cccc3c2sc2ccccc23)c1)c1cccc2c1oc1ccccc12]

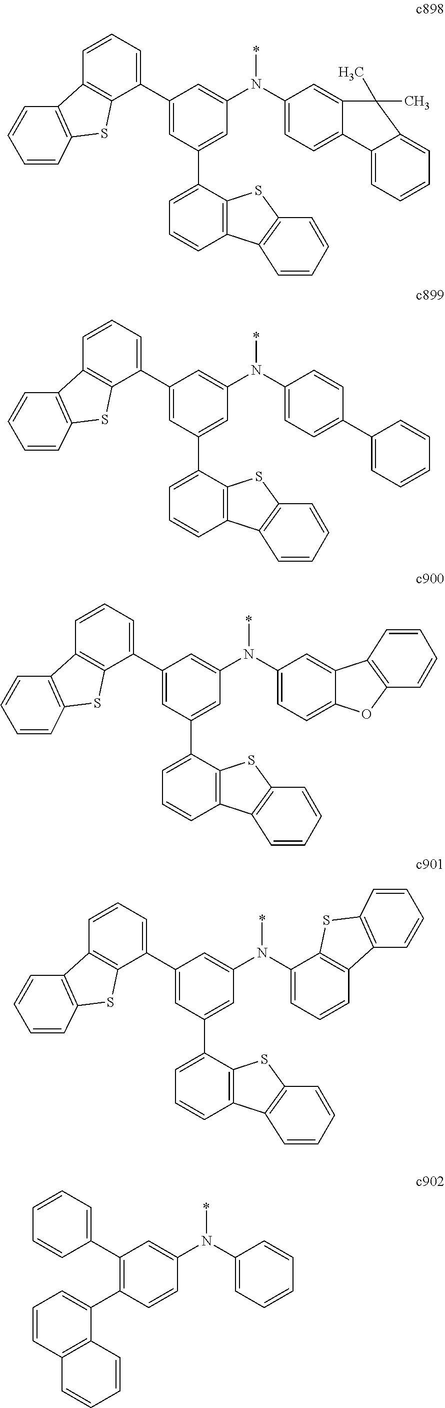 [SMILES: *N(c1cc(-c2cccc3c2sc2ccccc23)cc(-c2cccc3c2sc2ccccc23)c1)c1ccc2c(c1)C(C)(C)c1ccccc1-2 ; *N(c1ccc(-c2ccccc2)cc1)c1cc(-c2cccc3c2sc2ccccc23)cc(-c2cccc3c2sc2ccccc23)c1 ; *N(c1cc(-c2cccc3c2sc2ccccc23)cc(-c2cccc3c2sc2ccccc23)c1)c1ccc2oc3ccccc3c2c1 ; *N(c1cc(-c2cccc3c2sc2ccccc23)cc(-c2cccc3c2sc2ccccc23)c1)c1cccc2c1sc1ccccc12 ; *N(c1ccccc1)c1ccc(-c2cccc3ccccc23)c(-c2ccccc2)c1]

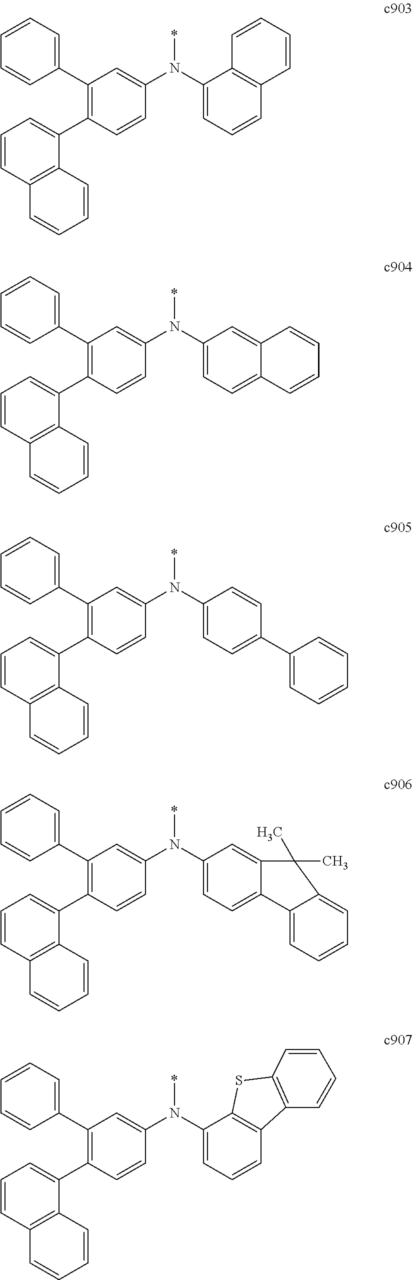 [SMILES: *N(c1ccc(-c2cccc3ccccc23)c(-c2ccccc2)c1)c1cccc2ccccc12 ; *N(c1ccc(-c2cccc3ccccc23)c(-c2ccccc2)c1)c1ccc2ccccc2c1 ; *N(c1ccc(-c2ccccc2)cc1)c1ccc(-c2cccc3ccccc23)c(-c2ccccc2)c1 ; *N(c1ccc(-c2cccc3ccccc23)c(-c2ccccc2)c1)c1ccc2c(c1)C(C)(C)c1ccccc1-2 ; *N(c1ccc(-c2cccc3ccccc23)c(-c2ccccc2)c1)c1cccc2c1sc1ccccc12]

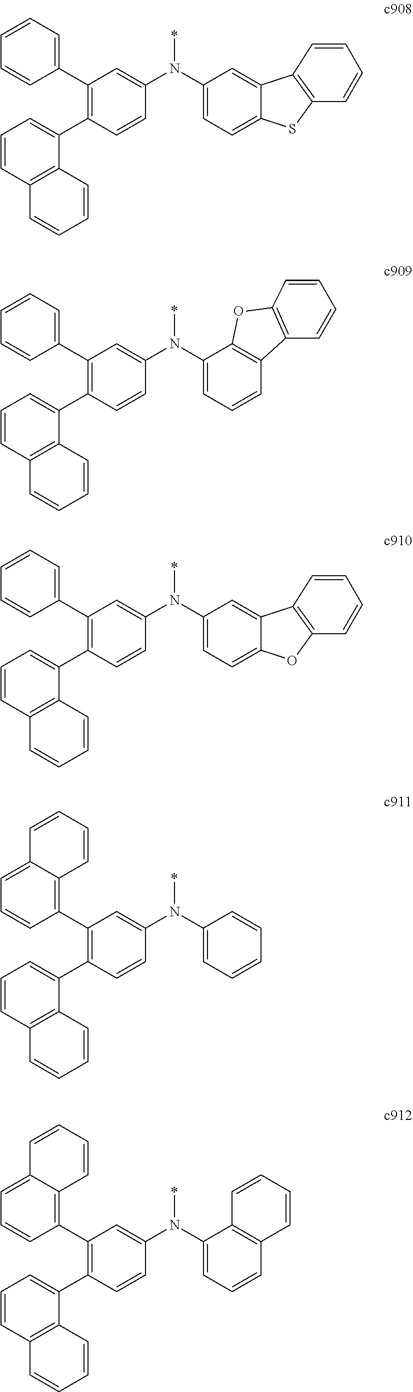

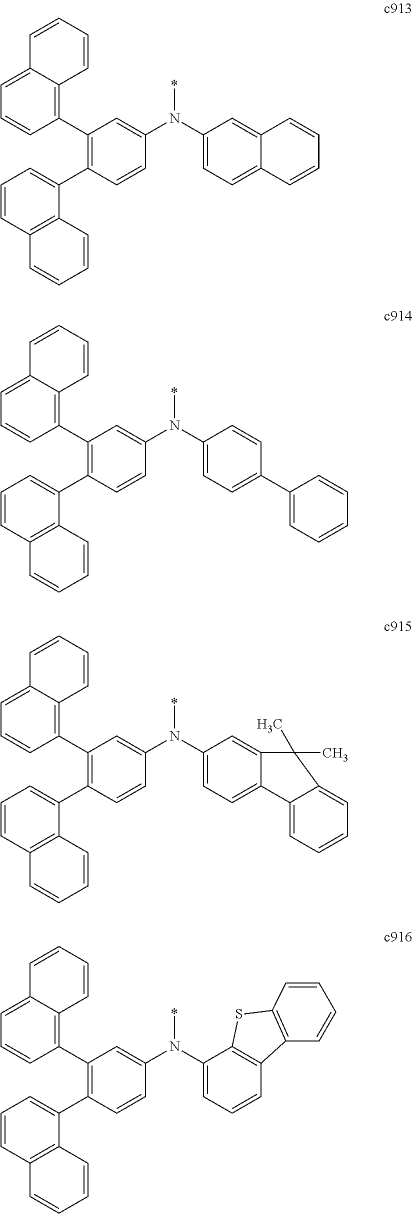

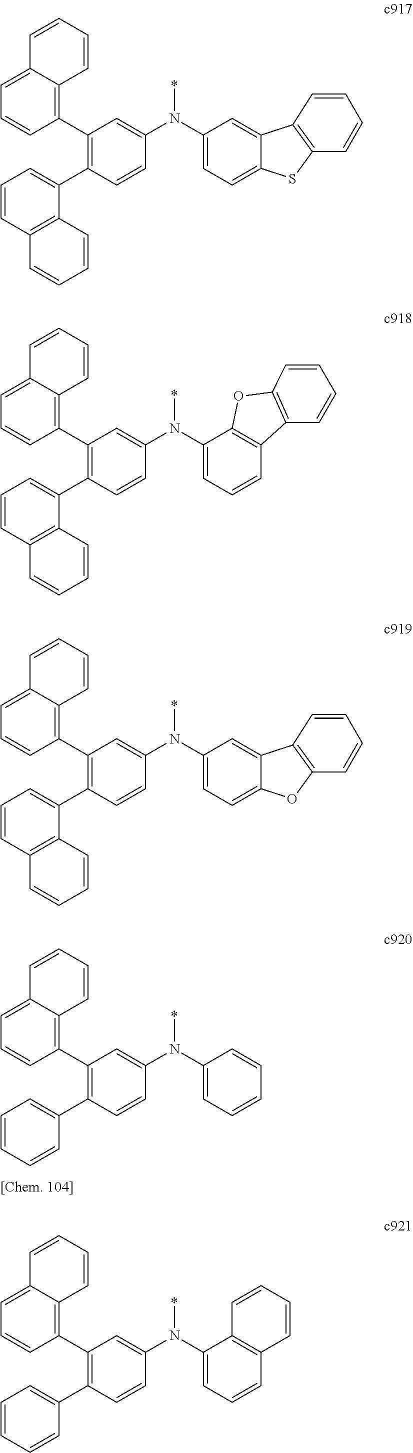

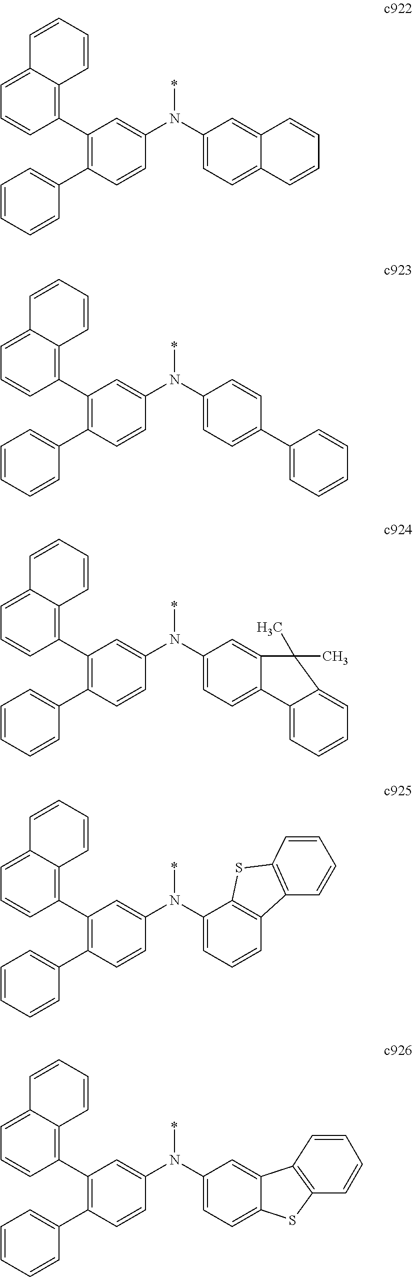 [SMILES: *N(c1ccc(-c2ccccc2)c(-c2cccc3ccccc23)c1)c1ccc2ccccc2c1 ; *N(c1ccc(-c2ccccc2)cc1)c1ccc(-c2ccccc2)c(-c2cccc3ccccc23)c1 ; *N(c1ccc(-c2ccccc2)c(-c2cccc3ccccc23)c1)c1ccc2c(c1)C(C)(C)c1ccccc1-2 ; *N(c1ccc(-c2ccccc2)c(-c2cccc3ccccc23)c1)c1cccc2c1sc1ccccc12 ; *N(c1ccc(-c2ccccc2)c(-c2cccc3ccccc23)c1)c1ccc2sc3ccccc3c2c1]

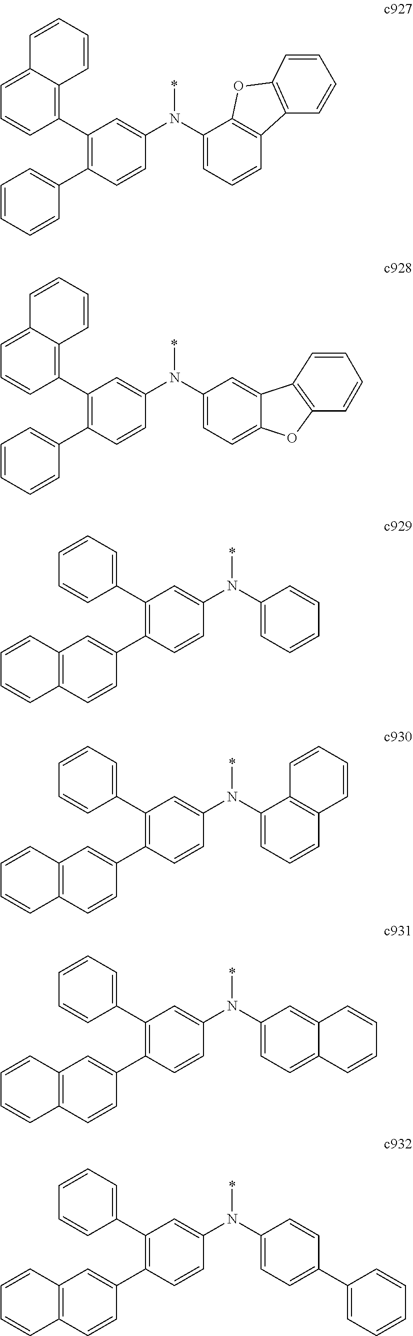

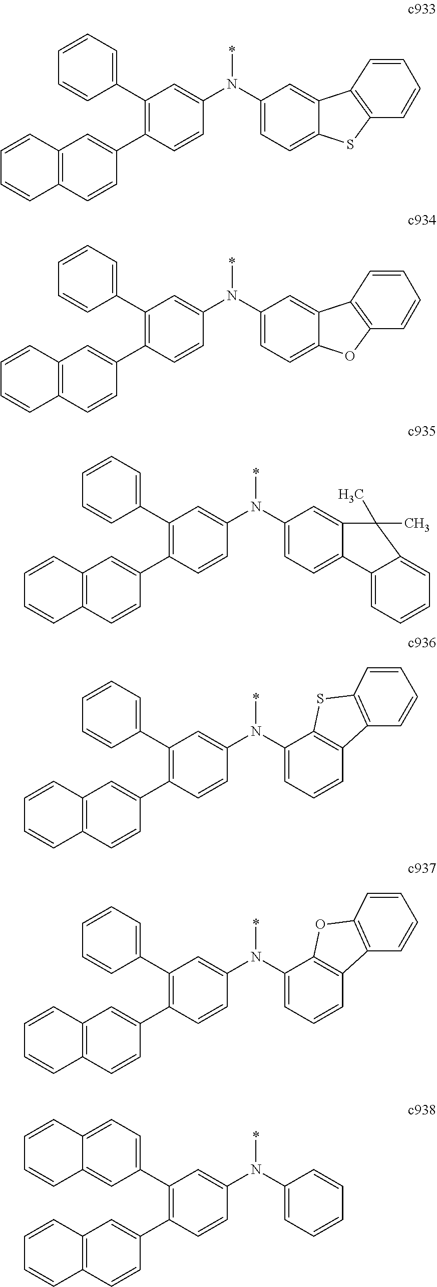

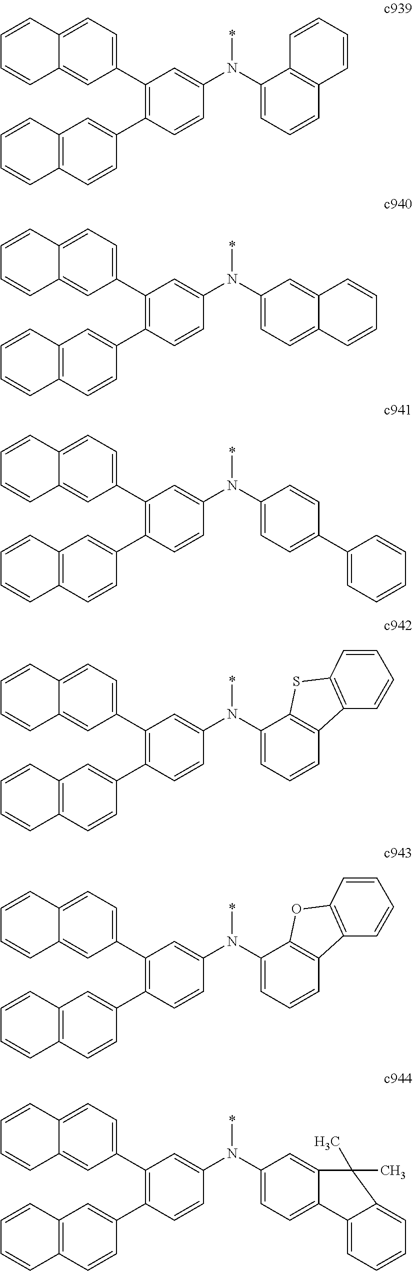

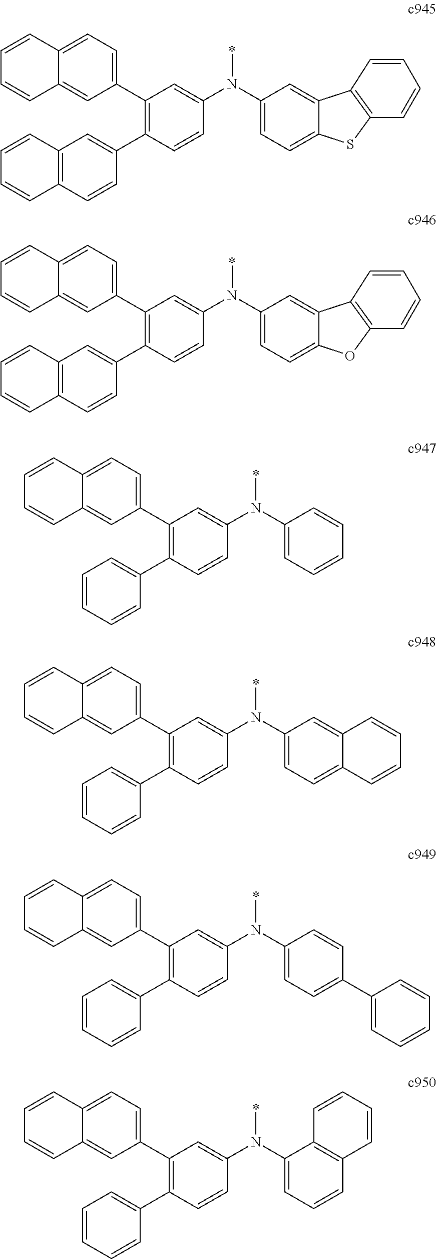

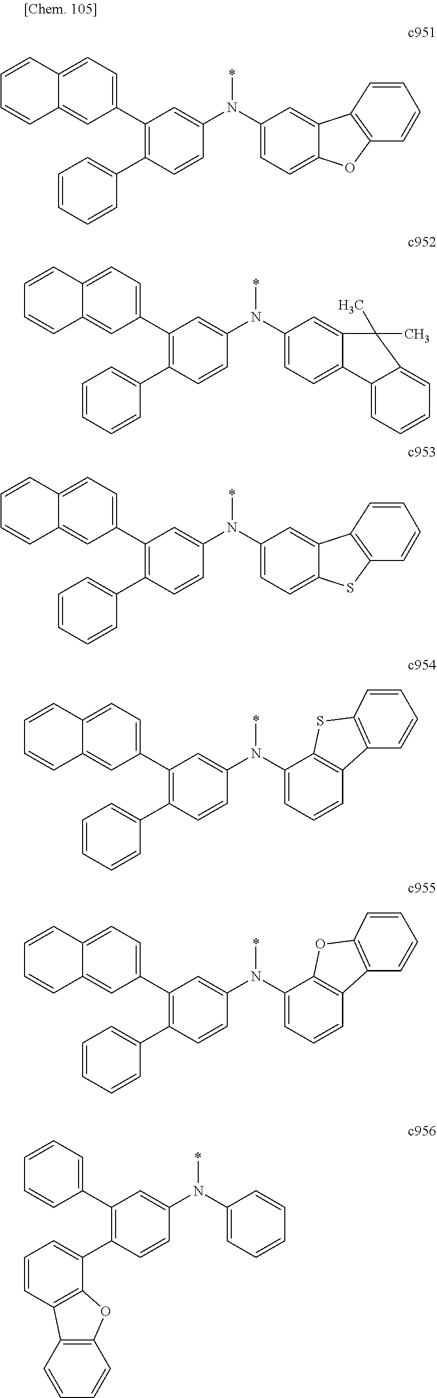 [SMILES: *N(c1ccc(-c2ccccc2)c(-c2ccc3ccccc3c2)c1)c1ccc2oc3ccccc3c2c1 ; *N(c1ccc(-c2ccccc2)c(-c2ccc3ccccc3c2)c1)c1ccc2c(c1)C(C)(C)c1ccccc1-2 ; *N(c1ccc(-c2ccccc2)c(-c2ccc3ccccc3c2)c1)c1ccc2sc3ccccc3c2c1 ; *N(c1ccc(-c2ccccc2)c(-c2ccc3ccccc3c2)c1)c1cccc2c1sc1ccccc12 ; *N(c1ccc(-c2ccccc2)c(-c2ccc3ccccc3c2)c1)c1cccc2c1oc1ccccc12 ; *N(c1ccccc1)c1ccc(-c2cccc3c2oc2ccccc23)c(-c2ccccc2)c1]

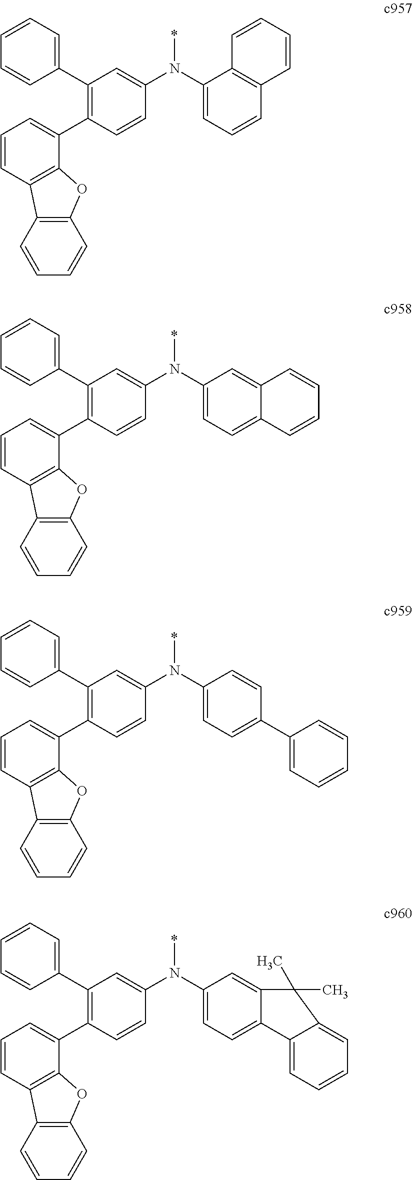

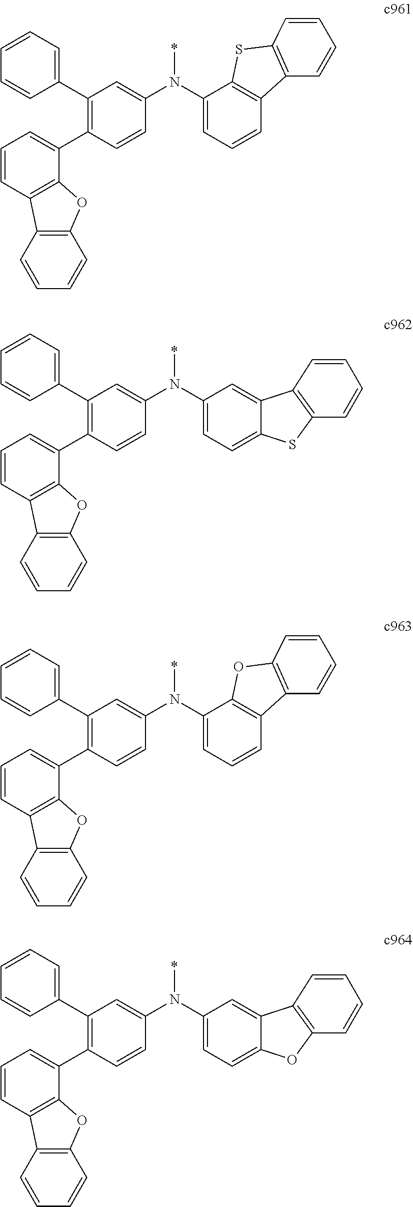

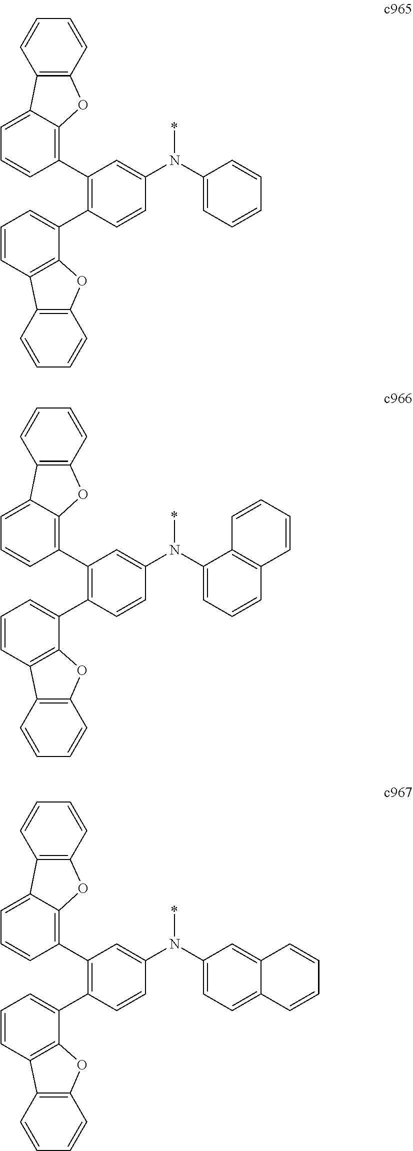

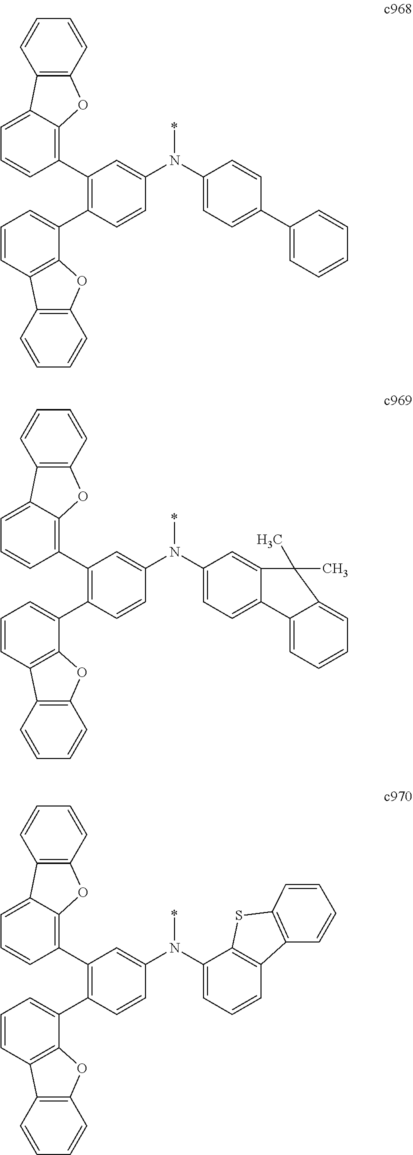

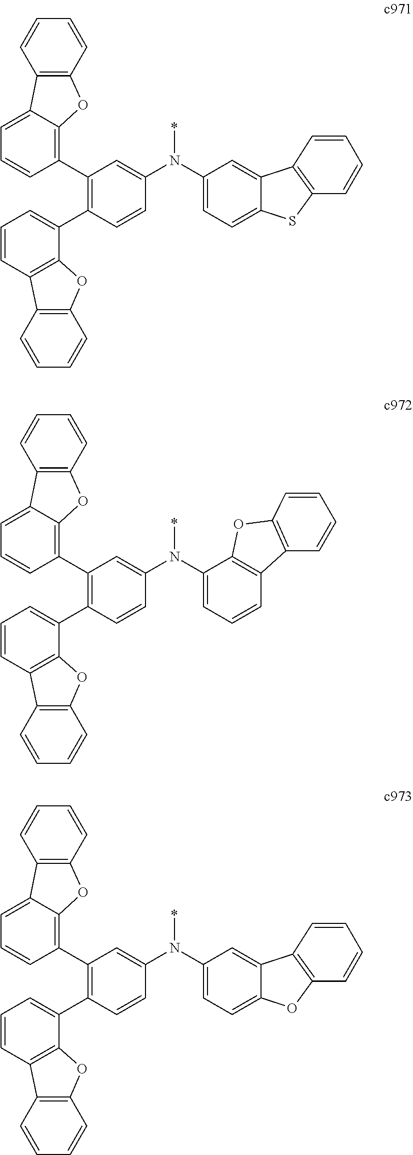

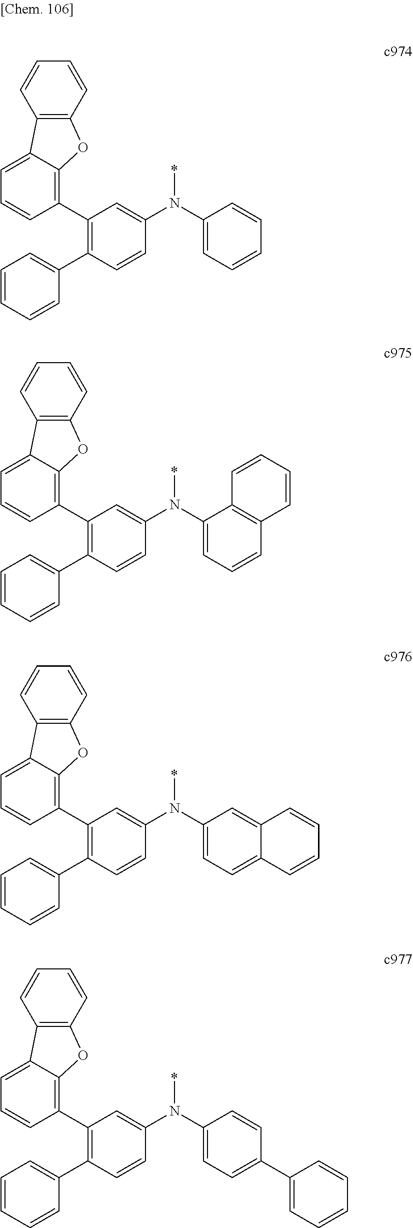

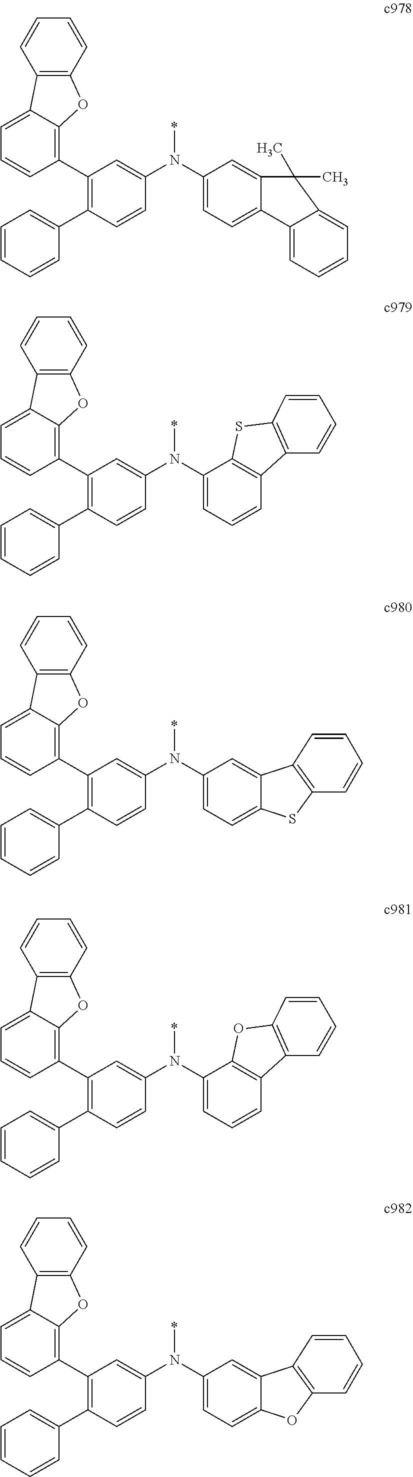

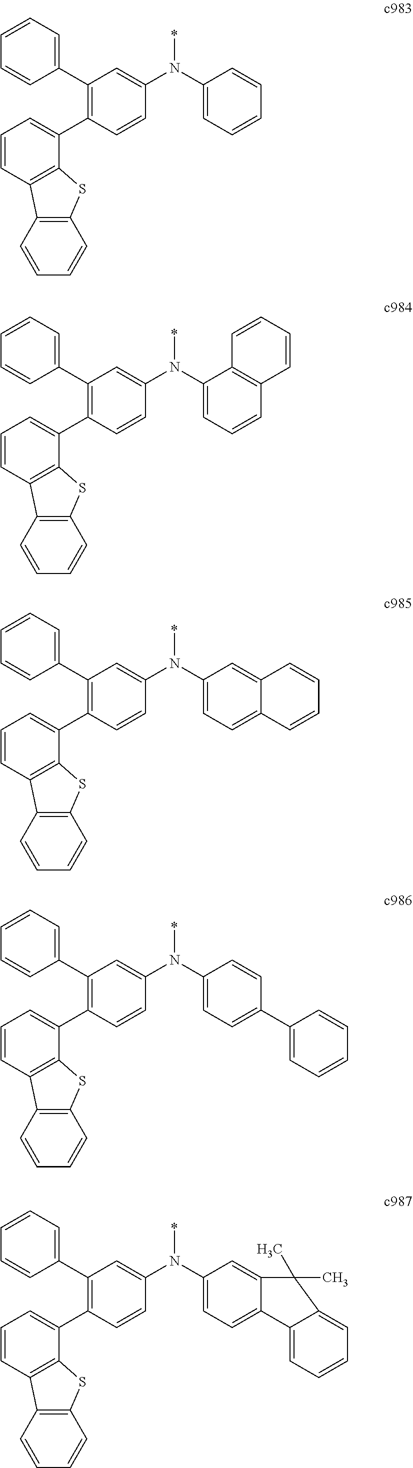

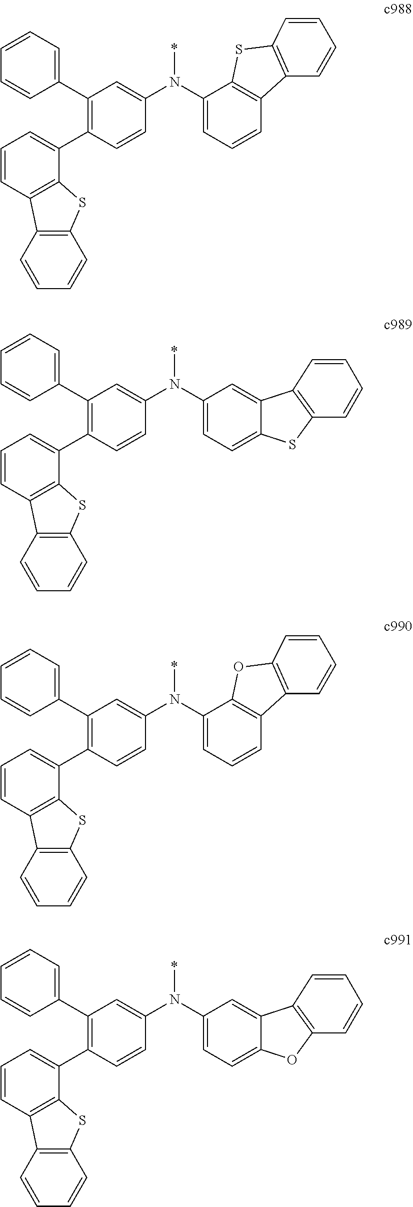

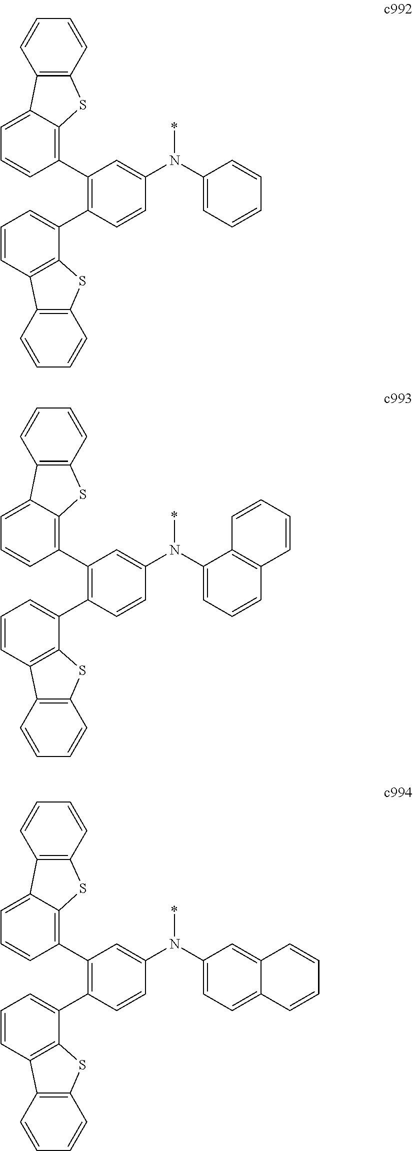

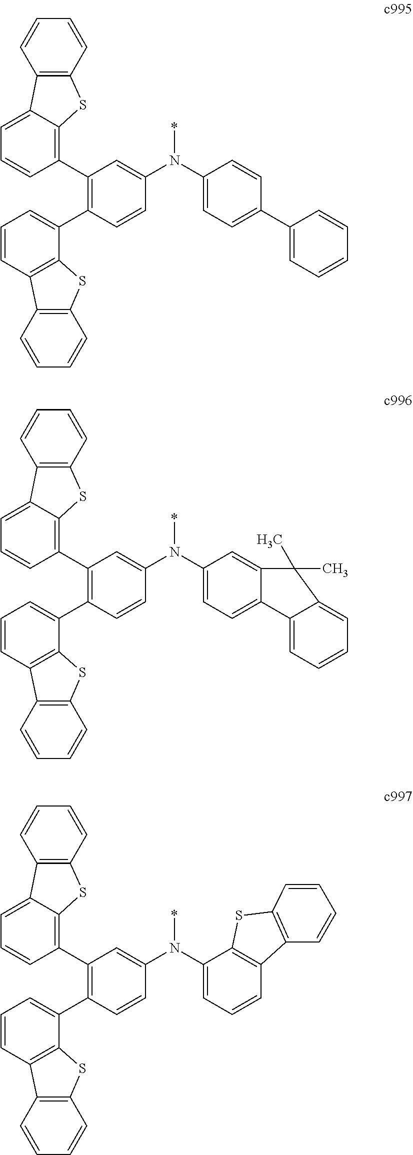

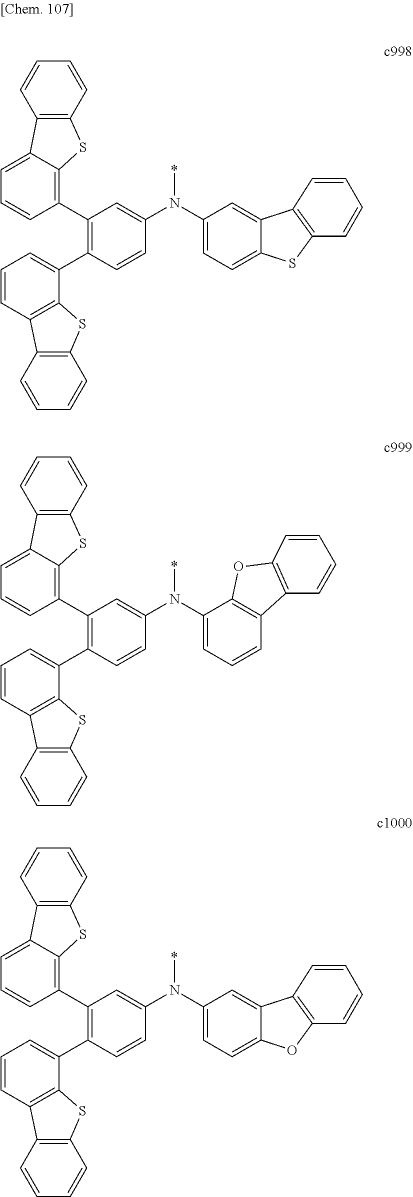

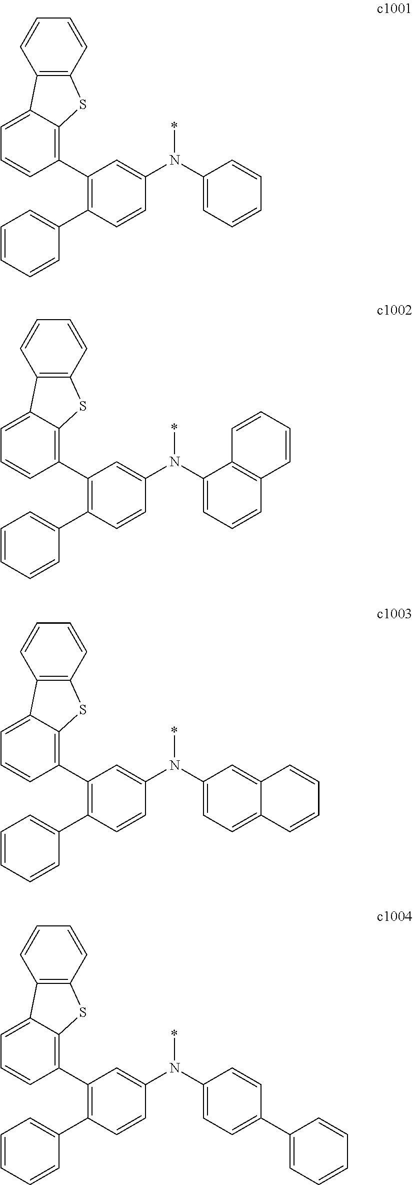

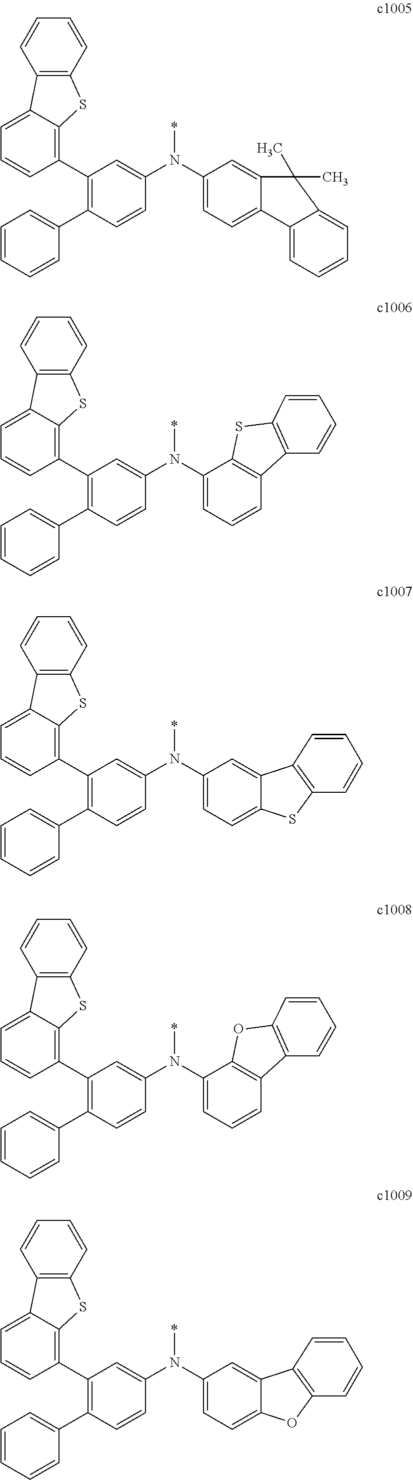

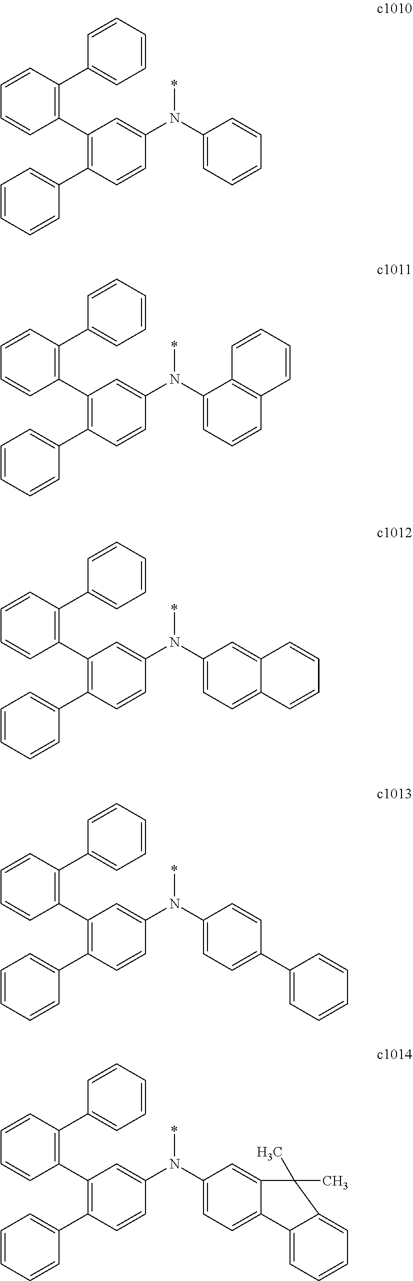 [SMILES: *N(c1ccccc1)c1ccc(-c2ccccc2)c(-c2ccccc2-c2ccccc2)c1 ; *N(c1ccc(-c2ccccc2)c(-c2ccccc2-c2ccccc2)c1)c1cccc2ccccc12 ; *N(c1ccc(-c2ccccc2)c(-c2ccccc2-c2ccccc2)c1)c1ccc2ccccc2c1 ; *N(c1ccc(-c2ccccc2)cc1)c1ccc(-c2ccccc2)c(-c2ccccc2-c2ccccc2)c1 ; *N(c1ccc(-c2ccccc2)c(-c2ccccc2-c2ccccc2)c1)c1ccc2c(c1)C(C)(C)c1ccccc1-2]

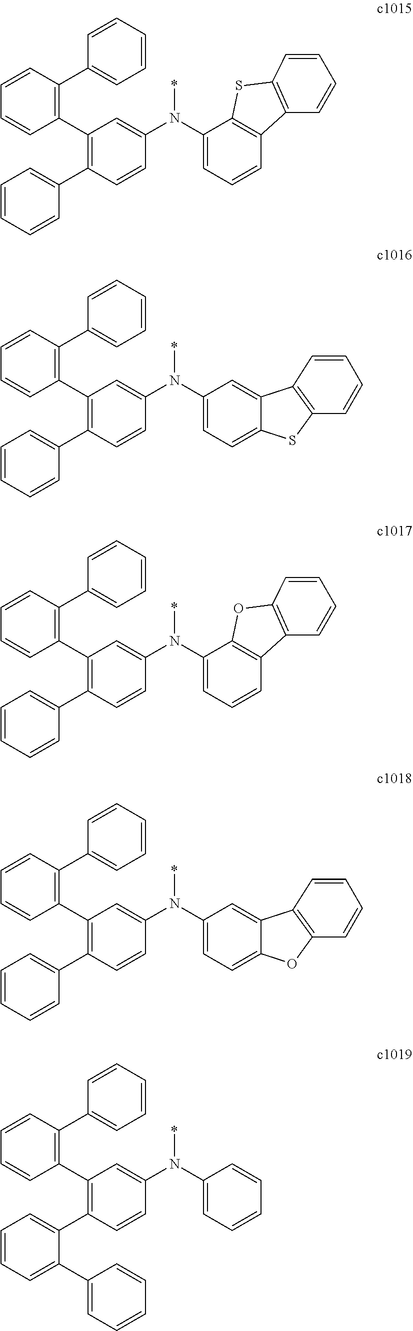 [SMILES: *N(c1ccc(-c2ccccc2)c(-c2ccccc2-c2ccccc2)c1)c1cccc2c1sc1ccccc12 ; *N(c1ccc(-c2ccccc2)c(-c2ccccc2-c2ccccc2)c1)c1ccc2sc3ccccc3c2c1 ; *N(c1ccc(-c2ccccc2)c(-c2ccccc2-c2ccccc2)c1)c1cccc2c1oc1ccccc12 ; *N(c1ccc(-c2ccccc2)c(-c2ccccc2-c2ccccc2)c1)c1ccc2oc3ccccc3c2c1 ; *N(c1ccccc1)c1ccc(-c2ccccc2-c2ccccc2)c(-c2ccccc2-c2ccccc2)c1]

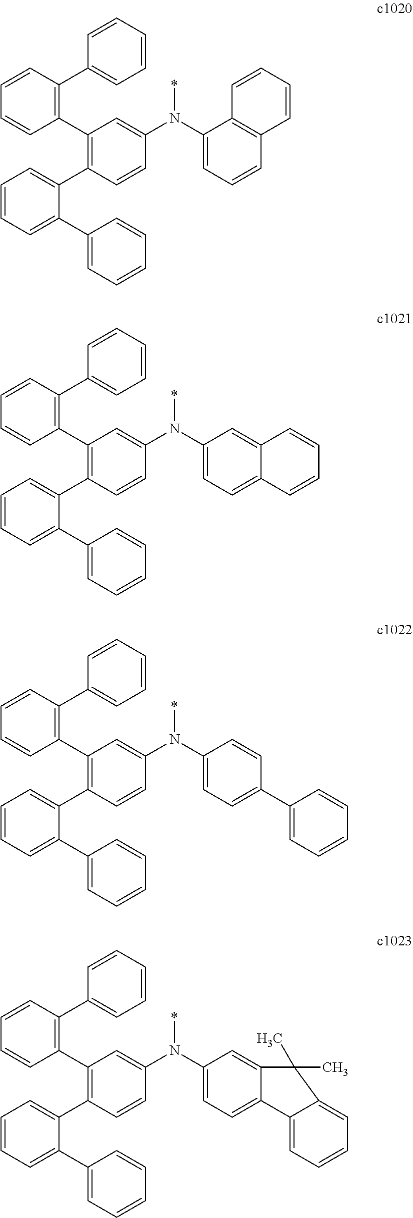

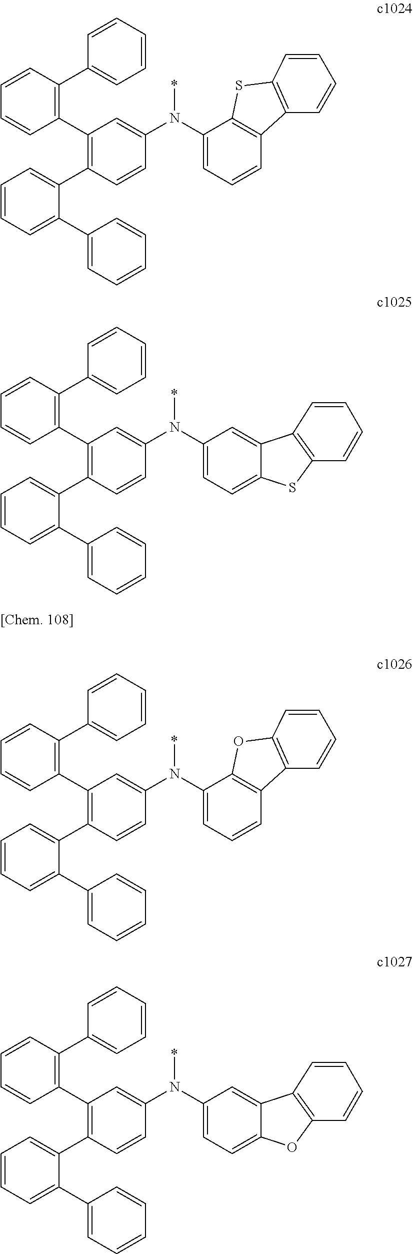

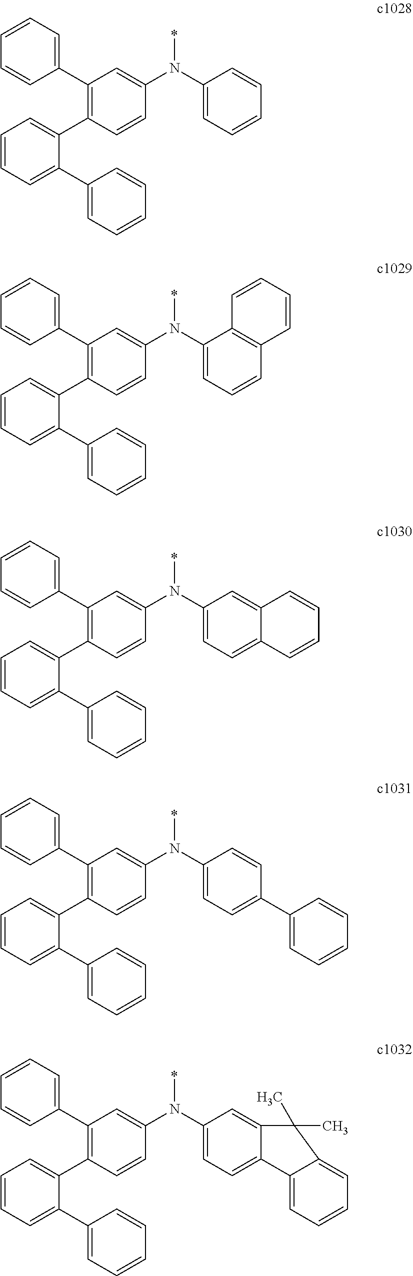 [SMILES: *N(c1ccccc1)c1ccc(-c2ccccc2-c2ccccc2)c(-c2ccccc2)c1 ; *N(c1ccc(-c2ccccc2-c2ccccc2)c(-c2ccccc2)c1)c1cccc2ccccc12 ; *N(c1ccc(-c2ccccc2-c2ccccc2)c(-c2ccccc2)c1)c1ccc2ccccc2c1 ; *N(c1ccc(-c2ccccc2)cc1)c1ccc(-c2ccccc2-c2ccccc2)c(-c2ccccc2)c1 ; *N(c1ccc(-c2ccccc2-c2ccccc2)c(-c2ccccc2)c1)c1ccc2c(c1)C(C)(C)c1ccccc1-2]

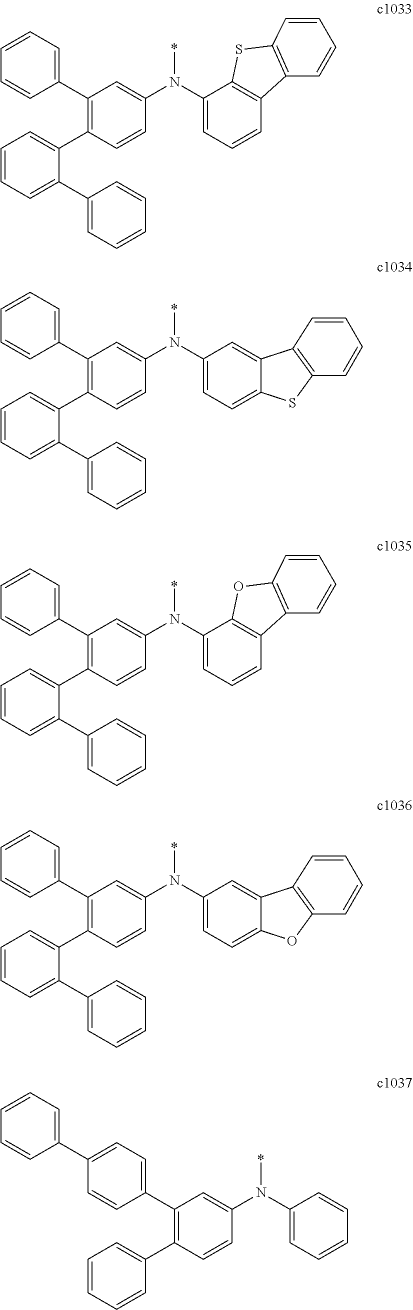 [SMILES: *N(c1ccc(-c2ccccc2-c2ccccc2)c(-c2ccccc2)c1)c1cccc2c1sc1ccccc12 ; *N(c1ccc(-c2ccccc2-c2ccccc2)c(-c2ccccc2)c1)c1ccc2sc3ccccc3c2c1 ; *N(c1ccc(-c2ccccc2-c2ccccc2)c(-c2ccccc2)c1)c1cccc2c1oc1ccccc12 ; *N(c1ccc(-c2ccccc2-c2ccccc2)c(-c2ccccc2)c1)c1ccc2oc3ccccc3c2c1 ; *N(c1ccccc1)c1ccc(-c2ccccc2)c(-c2ccc(-c3ccccc3)cc2)c1]

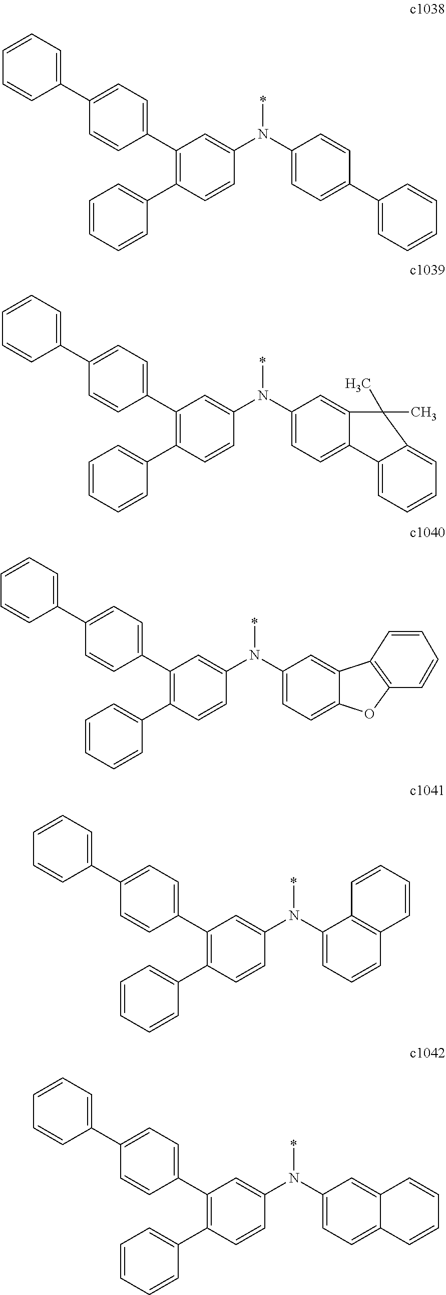 [SMILES: *N(c1ccc(-c2ccccc2)cc1)c1ccc(-c2ccccc2)c(-c2ccc(-c3ccccc3)cc2)c1 ; *N(c1ccc(-c2ccccc2)c(-c2ccc(-c3ccccc3)cc2)c1)c1ccc2c(c1)C(C)(C)c1ccccc1-2 ; *N(c1ccc(-c2ccccc2)c(-c2ccc(-c3ccccc3)cc2)c1)c1ccc2oc3ccccc3c2c1 ; *N(c1ccc(-c2ccccc2)c(-c2ccc(-c3ccccc3)cc2)c1)c1cccc2ccccc12 ; *N(c1ccc(-c2ccccc2)c(-c2ccc(-c3ccccc3)cc2)c1)c1ccc2ccccc2c1]

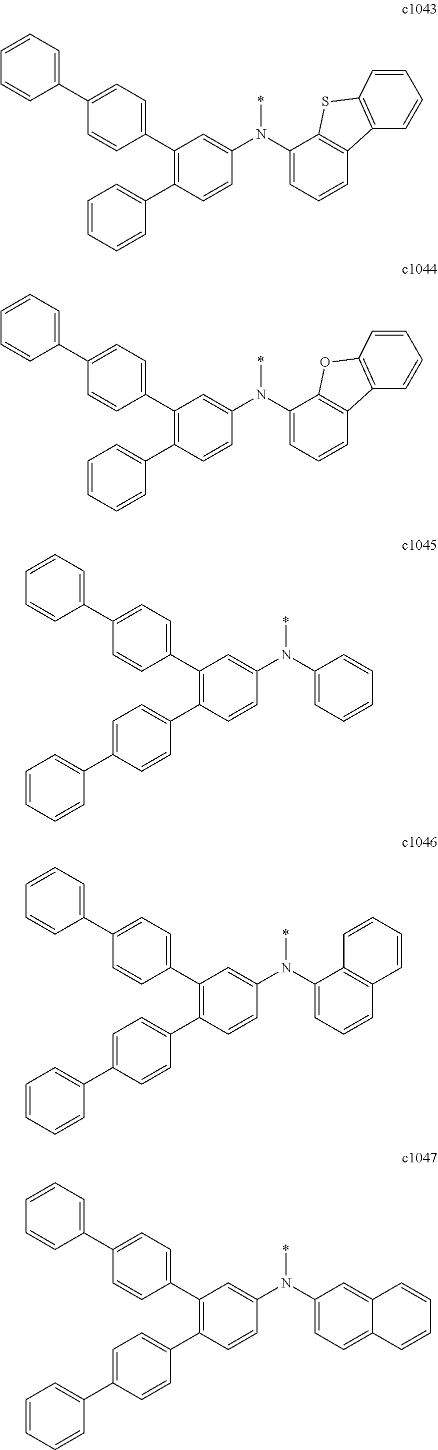

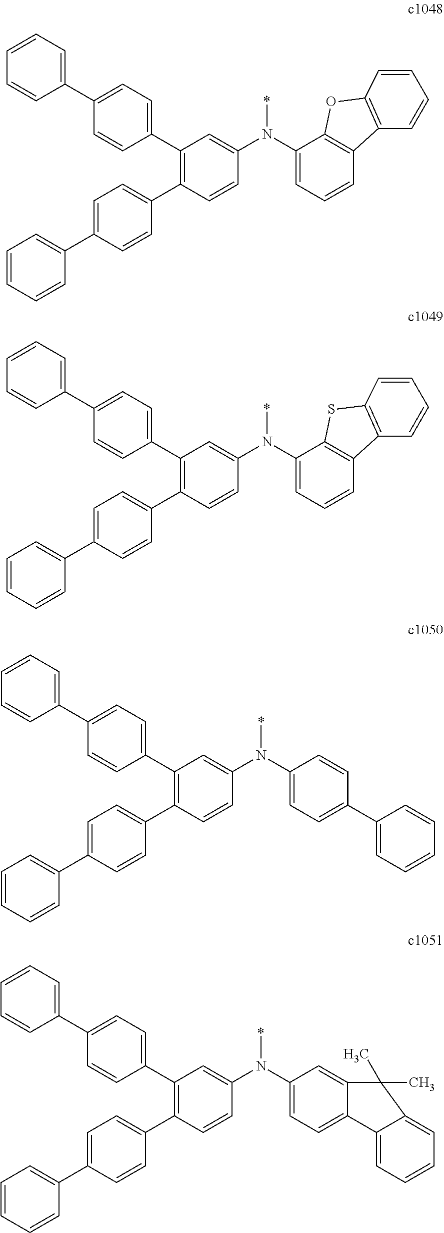

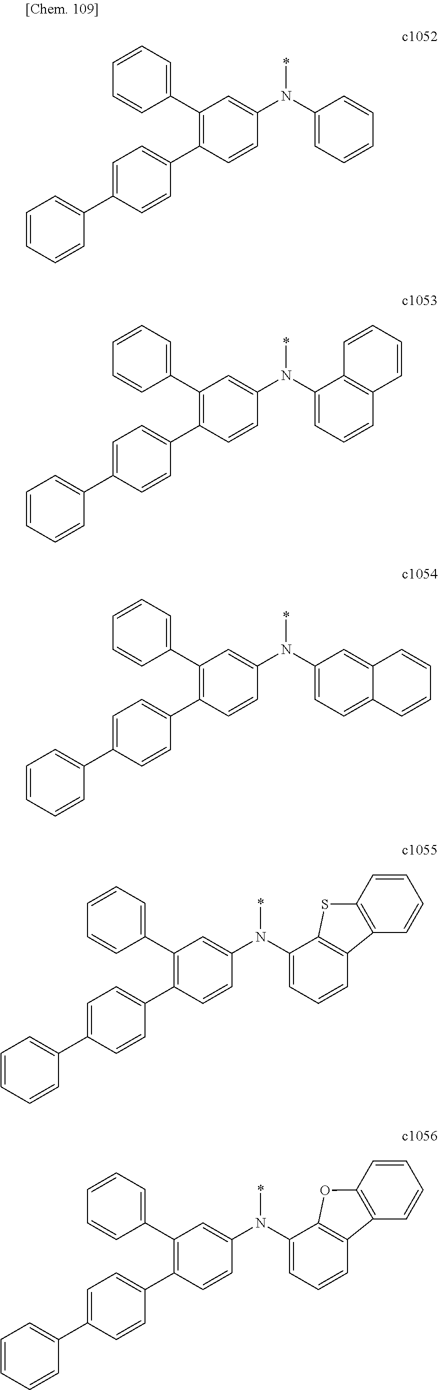 [SMILES: *N(c1ccccc1)c1ccc(-c2ccc(-c3ccccc3)cc2)c(-c2ccccc2)c1 ; *N(c1ccc(-c2ccc(-c3ccccc3)cc2)c(-c2ccccc2)c1)c1cccc2ccccc12 ; *N(c1ccc(-c2ccc(-c3ccccc3)cc2)c(-c2ccccc2)c1)c1ccc2ccccc2c1 ; *N(c1ccc(-c2ccc(-c3ccccc3)cc2)c(-c2ccccc2)c1)c1cccc2c1sc1ccccc12 ; *N(c1ccc(-c2ccc(-c3ccccc3)cc2)c(-c2ccccc2)c1)c1cccc2c1oc1ccccc12]

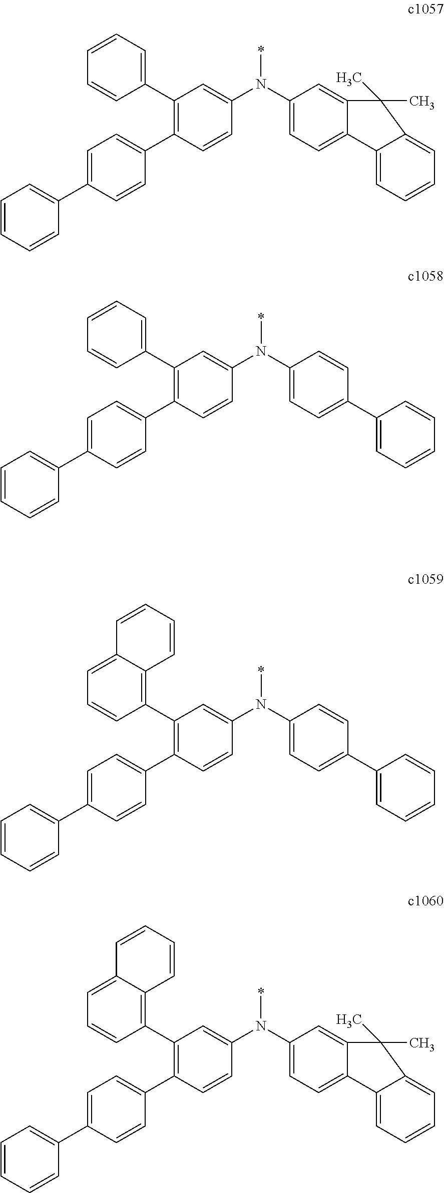 [SMILES: *N(c1ccc(-c2ccc(-c3ccccc3)cc2)c(-c2ccccc2)c1)c1ccc2c(c1)C(C)(C)c1ccccc1-2 ; *N(c1ccc(-c2ccccc2)cc1)c1ccc(-c2ccc(-c3ccccc3)cc2)c(-c2ccccc2)c1 ; *N(c1ccc(-c2ccccc2)cc1)c1ccc(-c2ccc(-c3ccccc3)cc2)c(-c2cccc3ccccc23)c1 ; *N(c1ccc(-c2ccc(-c3ccccc3)cc2)c(-c2cccc3ccccc23)c1)c1ccc2c(c1)C(C)(C)c1ccccc1-2]

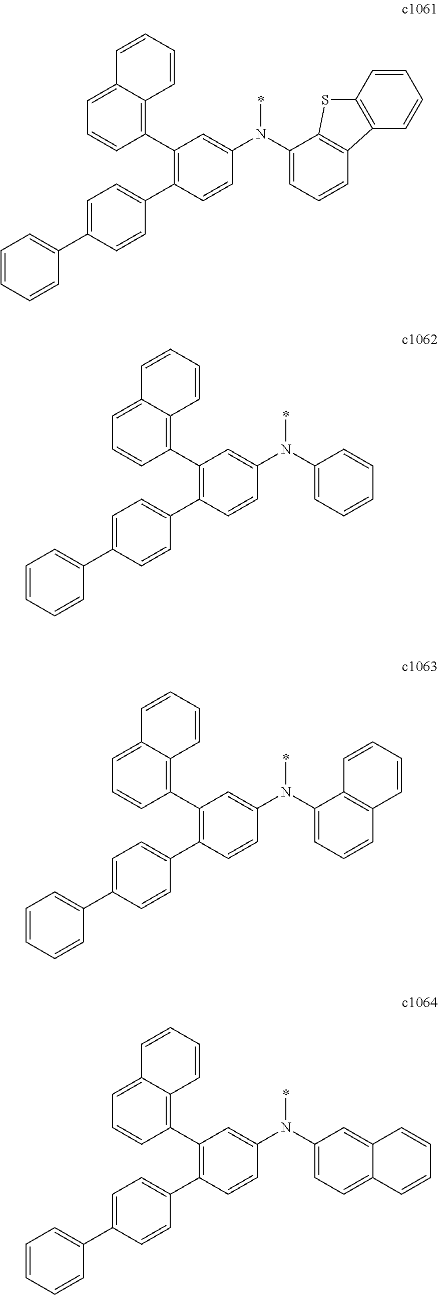 [SMILES: *N(c1ccc(-c2ccc(-c3ccccc3)cc2)c(-c2cccc3ccccc23)c1)c1cccc2c1sc1ccccc12 ; *N(c1ccccc1)c1ccc(-c2ccc(-c3ccccc3)cc2)c(-c2cccc3ccccc23)c1 ; *N(c1ccc(-c2ccc(-c3ccccc3)cc2)c(-c2cccc3ccccc23)c1)c1cccc2ccccc12 ; *N(c1ccc(-c2ccc(-c3ccccc3)cc2)c(-c2cccc3ccccc23)c1)c1ccc2ccccc2c1]

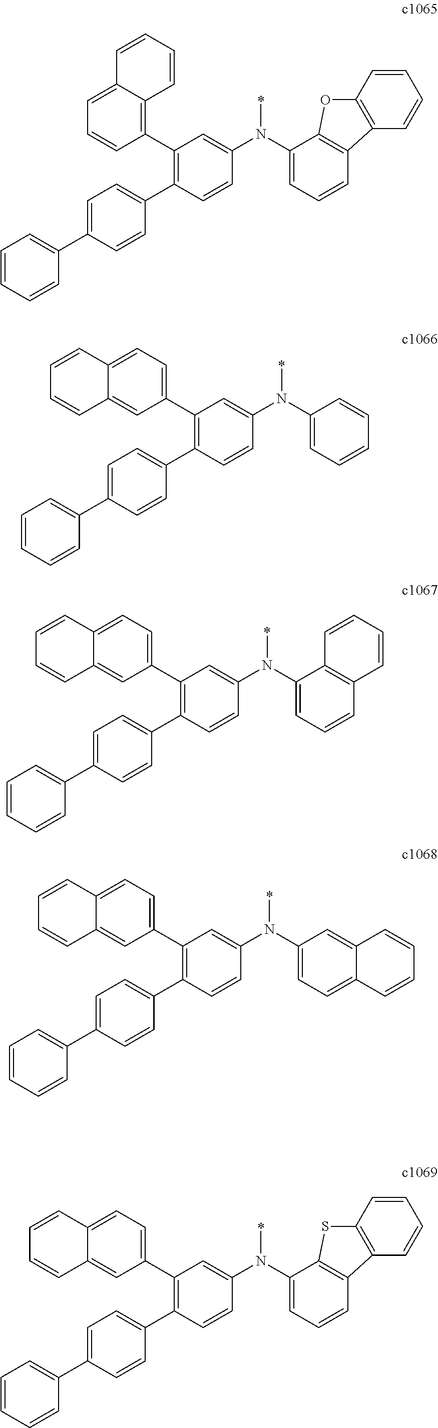 [SMILES: *N(c1ccc(-c2ccc(-c3ccccc3)cc2)c(-c2cccc3ccccc23)c1)c1cccc2c1oc1ccccc12 ; *N(c1ccccc1)c1ccc(-c2ccc(-c3ccccc3)cc2)c(-c2ccc3ccccc3c2)c1 ; *N(c1ccc(-c2ccc(-c3ccccc3)cc2)c(-c2ccc3ccccc3c2)c1)c1cccc2ccccc12 ; *N(c1ccc(-c2ccc(-c3ccccc3)cc2)c(-c2ccc3ccccc3c2)c1)c1ccc2ccccc2c1 ; *N(c1ccc(-c2ccc(-c3ccccc3)cc2)c(-c2ccc3ccccc3c2)c1)c1cccc2c1sc1ccccc12]

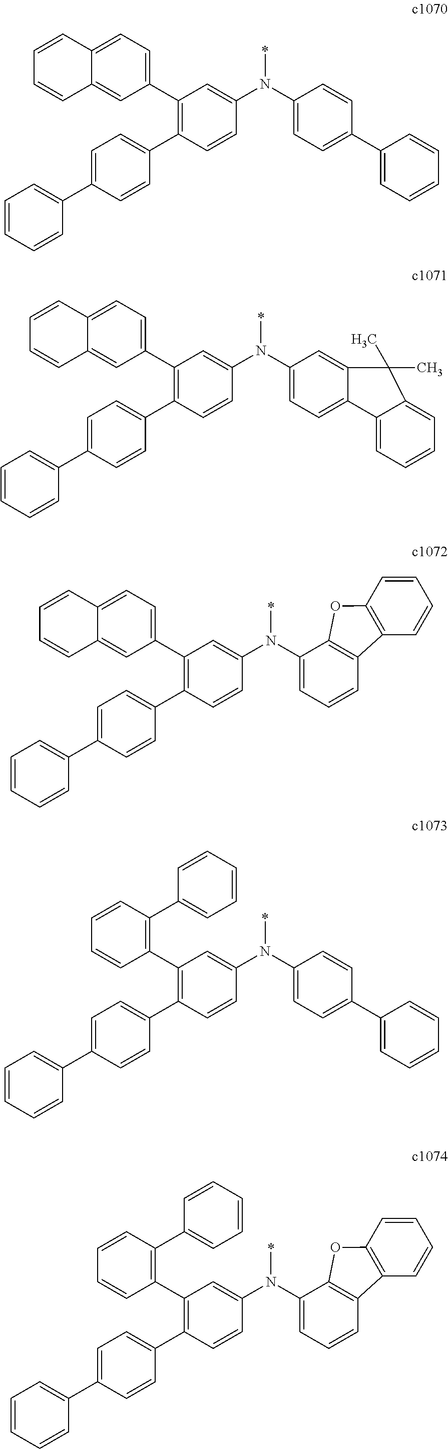 [SMILES: *N(c1ccc(-c2ccccc2)cc1)c1ccc(-c2ccc(-c3ccccc3)cc2)c(-c2ccc3ccccc3c2)c1 ; *N(c1ccc(-c2ccc(-c3ccccc3)cc2)c(-c2ccc3ccccc3c2)c1)c1ccc2c(c1)C(C)(C)c1ccccc1-2 ; *N(c1ccc(-c2ccc(-c3ccccc3)cc2)c(-c2ccc3ccccc3c2)c1)c1cccc2c1oc1ccccc12 ; *N(c1ccc(-c2ccccc2)cc1)c1ccc(-c2ccc(-c3ccccc3)cc2)c(-c2ccccc2-c2ccccc2)c1 ; *N(c1ccc(-c2ccc(-c3ccccc3)cc2)c(-c2ccccc2-c2ccccc2)c1)c1cccc2c1oc1ccccc12]

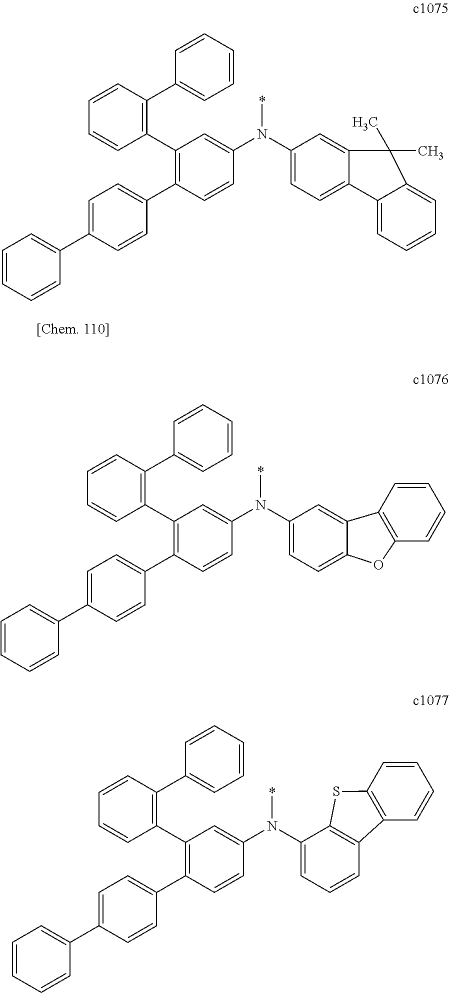 [SMILES: *N(c1ccc(-c2ccc(-c3ccccc3)cc2)c(-c2ccccc2-c2ccccc2)c1)c1ccc2c(c1)C(C)(C)c1ccccc1-2 ; *N(c1ccc(-c2ccc(-c3ccccc3)cc2)c(-c2ccccc2-c2ccccc2)c1)c1ccc2oc3ccccc3c2c1 ; *N(c1ccc(-c2ccc(-c3ccccc3)cc2)c(-c2ccccc2-c2ccccc2)c1)c1cccc2c1sc1ccccc12]

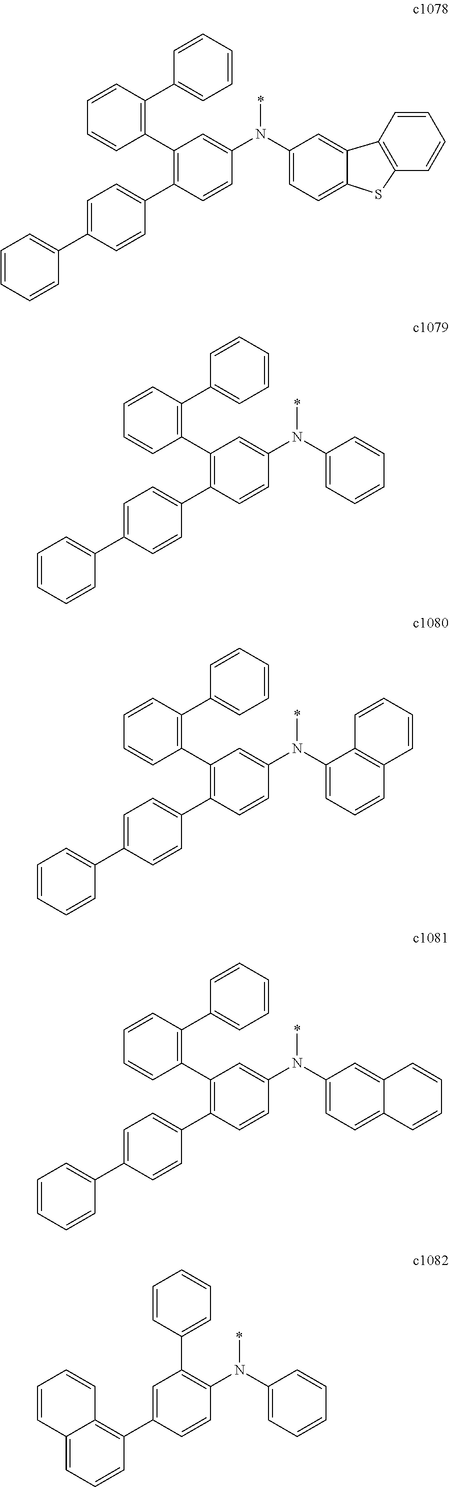 [SMILES: *N(c1ccc(-c2ccc(-c3ccccc3)cc2)c(-c2ccccc2-c2ccccc2)c1)c1ccc2sc3ccccc3c2c1 ; *N(c1ccccc1)c1ccc(-c2ccc(-c3ccccc3)cc2)c(-c2ccccc2-c2ccccc2)c1 ; *N(c1ccc(-c2ccc(-c3ccccc3)cc2)c(-c2ccccc2-c2ccccc2)c1)c1cccc2ccccc12 ; *N(c1ccc(-c2ccc(-c3ccccc3)cc2)c(-c2ccccc2-c2ccccc2)c1)c1ccc2ccccc2c1 ; *N(c1ccccc1)c1ccc(-c2cccc3ccccc23)cc1-c1ccccc1]

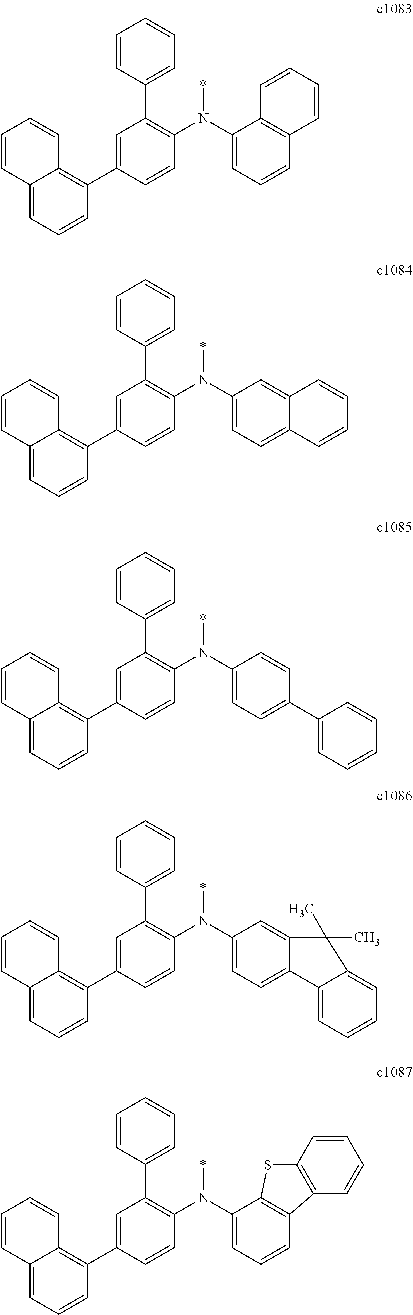 [SMILES: *N(c1ccc(-c2cccc3ccccc23)cc1-c1ccccc1)c1cccc2ccccc12 ; *N(c1ccc2ccccc2c1)c1ccc(-c2cccc3ccccc23)cc1-c1ccccc1 ; *N(c1ccc(-c2ccccc2)cc1)c1ccc(-c2cccc3ccccc23)cc1-c1ccccc1 ; *N(c1ccc2c(c1)C(C)(C)c1ccccc1-2)c1ccc(-c2cccc3ccccc23)cc1-c1ccccc1 ; *N(c1ccc(-c2cccc3ccccc23)cc1-c1ccccc1)c1cccc2c1sc1ccccc12]

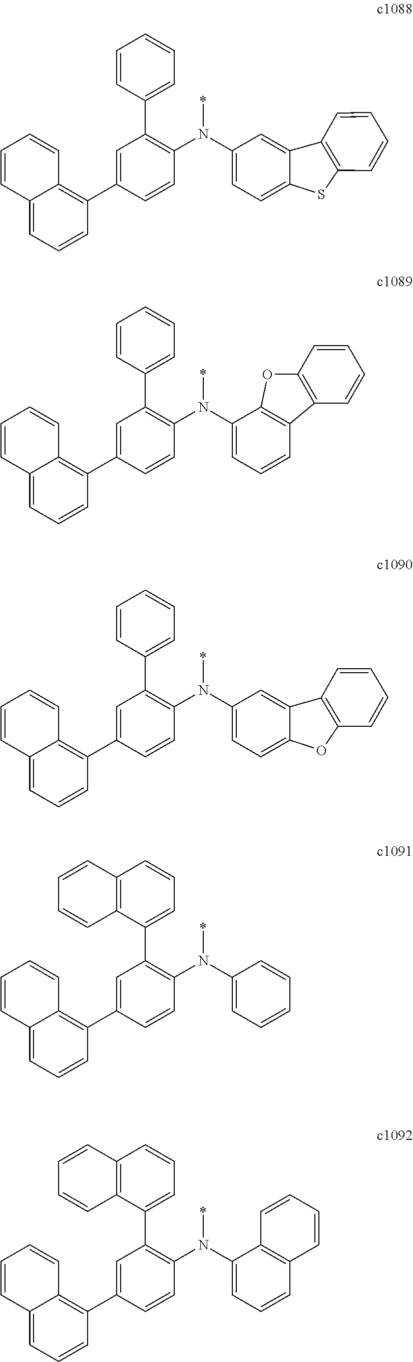 [SMILES: *N(c1ccc2sc3ccccc3c2c1)c1ccc(-c2cccc3ccccc23)cc1-c1ccccc1 ; *N(c1ccc(-c2cccc3ccccc23)cc1-c1ccccc1)c1cccc2c1oc1ccccc12 ; *N(c1ccc2oc3ccccc3c2c1)c1ccc(-c2cccc3ccccc23)cc1-c1ccccc1 ; *N(c1ccccc1)c1ccc(-c2cccc3ccccc23)cc1-c1cccc2ccccc12 ; *N(c1ccc(-c2cccc3ccccc23)cc1-c1cccc2ccccc12)c1cccc2ccccc12]

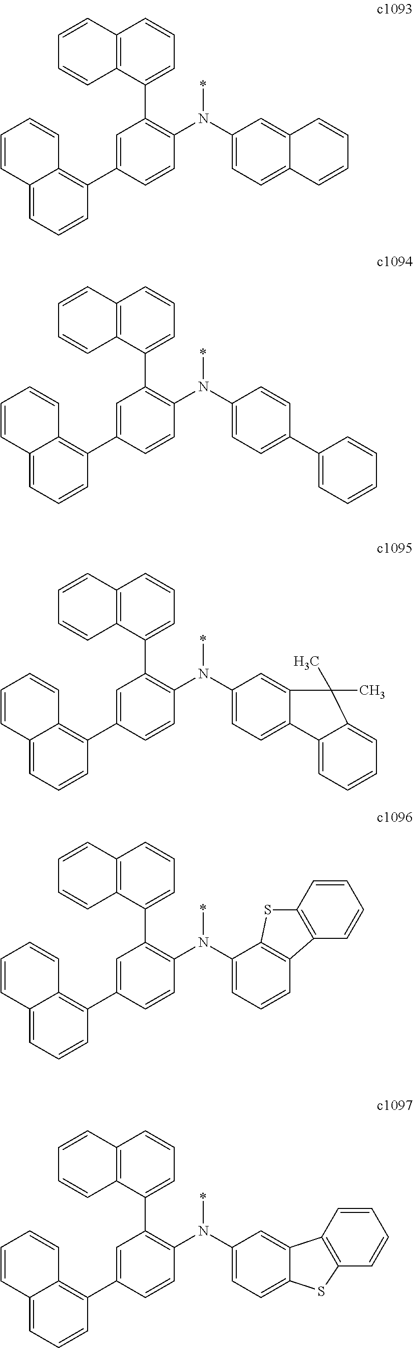 [SMILES: *N(c1ccc2ccccc2c1)c1ccc(-c2cccc3ccccc23)cc1-c1cccc2ccccc12 ; *N(c1ccc(-c2ccccc2)cc1)c1ccc(-c2cccc3ccccc23)cc1-c1cccc2ccccc12 ; *N(c1ccc2c(c1)C(C)(C)c1ccccc1-2)c1ccc(-c2cccc3ccccc23)cc1-c1cccc2ccccc12 ; *N(c1ccc(-c2cccc3ccccc23)cc1-c1cccc2ccccc12)c1cccc2c1sc1ccccc12 ; *N(c1ccc2sc3ccccc3c2c1)c1ccc(-c2cccc3ccccc23)cc1-c1cccc2ccccc12]

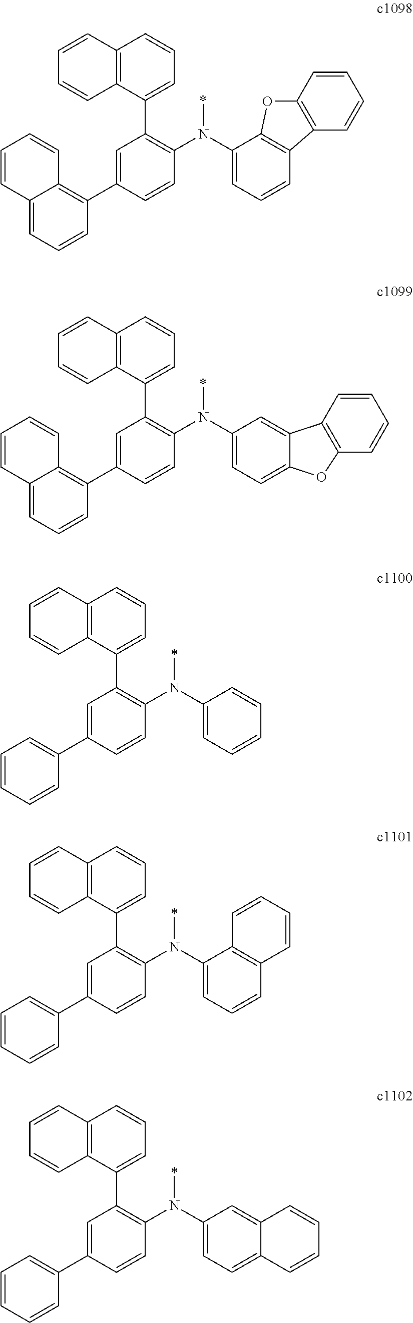 [SMILES: *N(c1ccc(-c2cccc3ccccc23)cc1-c1cccc2ccccc12)c1cccc2c1oc1ccccc12 ; *N(c1ccc2oc3ccccc3c2c1)c1ccc(-c2cccc3ccccc23)cc1-c1cccc2ccccc12 ; *N(c1ccccc1)c1ccc(-c2ccccc2)cc1-c1cccc2ccccc12 ; *N(c1ccc(-c2ccccc2)cc1-c1cccc2ccccc12)c1cccc2ccccc12 ; *N(c1ccc2ccccc2c1)c1ccc(-c2ccccc2)cc1-c1cccc2ccccc12]

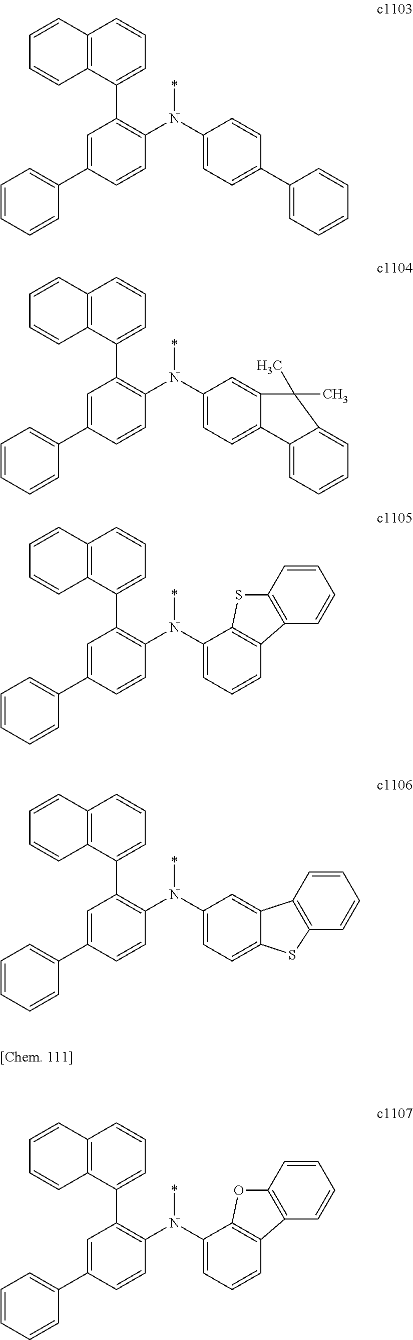

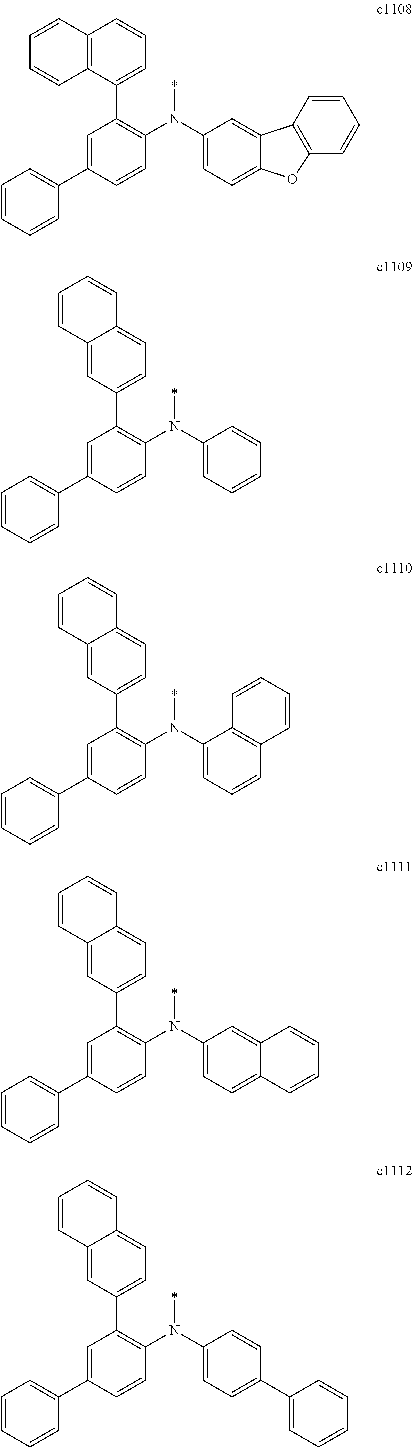 [SMILES: *N(c1ccc2oc3ccccc3c2c1)c1ccc(-c2ccccc2)cc1-c1cccc2ccccc12 ; *N(c1ccccc1)c1ccc(-c2ccccc2)cc1-c1ccc2ccccc2c1 ; *N(c1ccc(-c2ccccc2)cc1-c1ccc2ccccc2c1)c1cccc2ccccc12 ; *N(c1ccc2ccccc2c1)c1ccc(-c2ccccc2)cc1-c1ccc2ccccc2c1 ; *N(c1ccc(-c2ccccc2)cc1)c1ccc(-c2ccccc2)cc1-c1ccc2ccccc2c1]

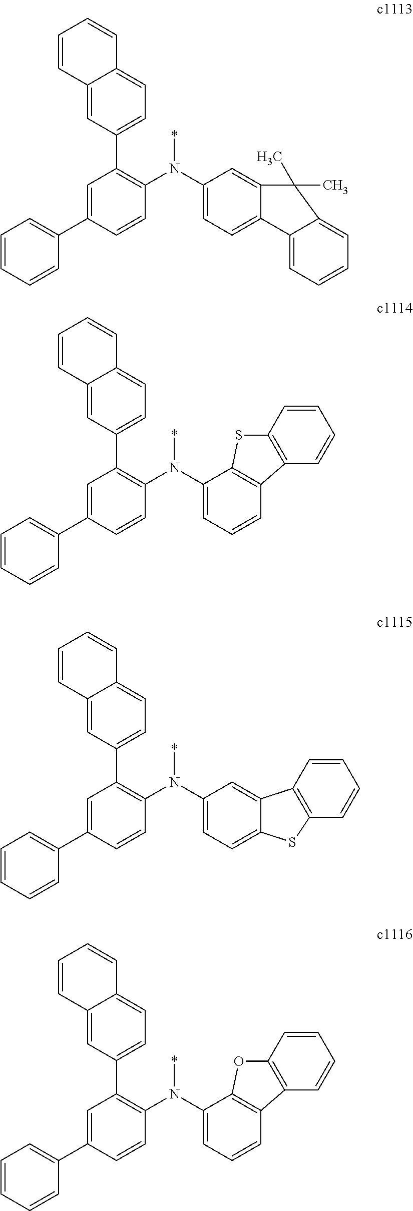 [SMILES: *N(c1ccc2c(c1)C(C)(C)c1ccccc1-2)c1ccc(-c2ccccc2)cc1-c1ccc2ccccc2c1 ; *N(c1ccc(-c2ccccc2)cc1-c1ccc2ccccc2c1)c1cccc2c1sc1ccccc12 ; *N(c1ccc2sc3ccccc3c2c1)c1ccc(-c2ccccc2)cc1-c1ccc2ccccc2c1 ; *N(c1ccc(-c2ccccc2)cc1-c1ccc2ccccc2c1)c1cccc2c1oc1ccccc12]

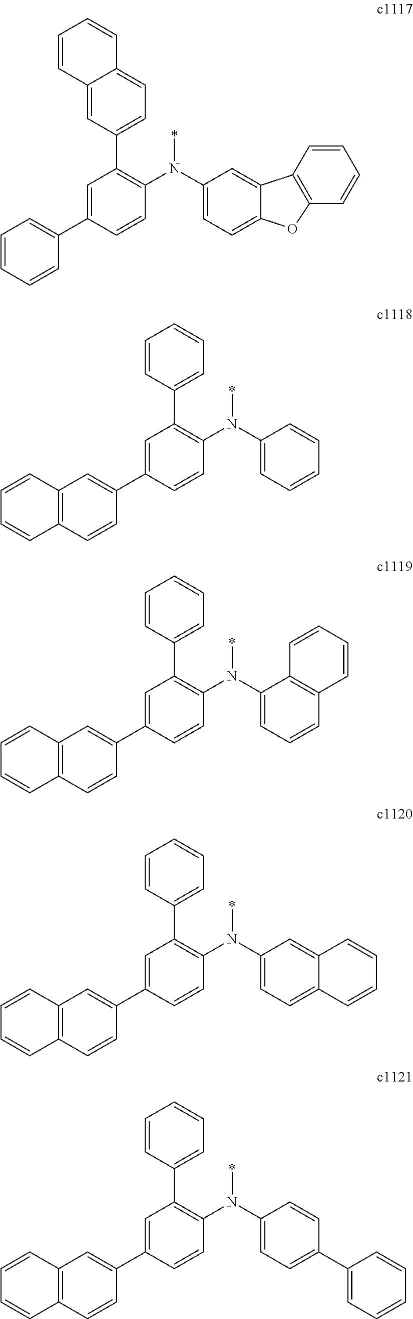 [SMILES: *N(c1ccc2oc3ccccc3c2c1)c1ccc(-c2ccccc2)cc1-c1ccc2ccccc2c1 ; *N(c1ccccc1)c1ccc(-c2ccc3ccccc3c2)cc1-c1ccccc1 ; *N(c1ccc(-c2ccc3ccccc3c2)cc1-c1ccccc1)c1cccc2ccccc12 ; *N(c1ccc2ccccc2c1)c1ccc(-c2ccc3ccccc3c2)cc1-c1ccccc1 ; *N(c1ccc(-c2ccccc2)cc1)c1ccc(-c2ccc3ccccc3c2)cc1-c1ccccc1]

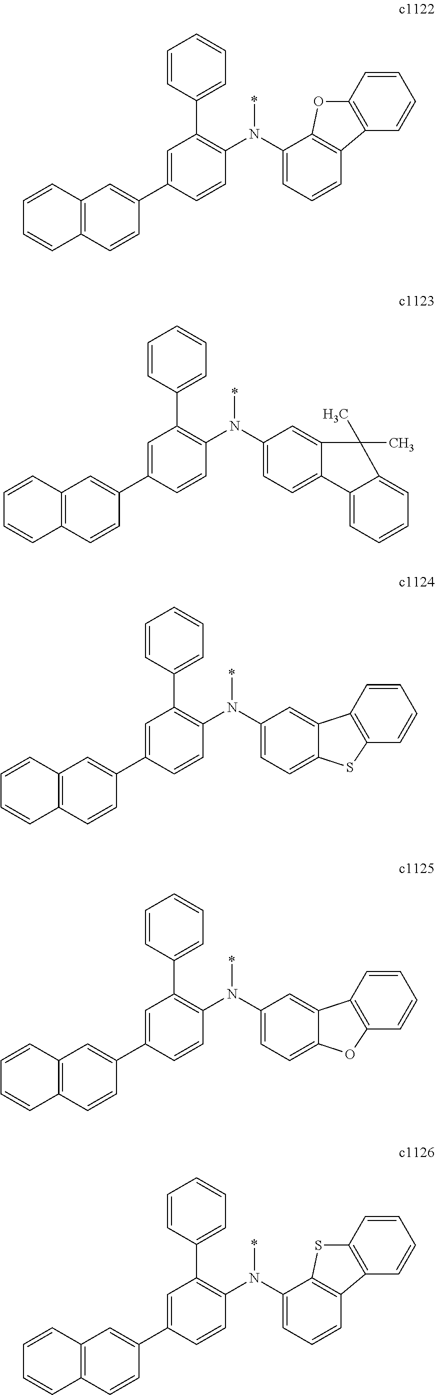 [SMILES: *N(c1ccc(-c2ccc3ccccc3c2)cc1-c1ccccc1)c1cccc2c1oc1ccccc12 ; *N(c1ccc2c(c1)C(C)(C)c1ccccc1-2)c1ccc(-c2ccc3ccccc3c2)cc1-c1ccccc1 ; *N(c1ccc2sc3ccccc3c2c1)c1ccc(-c2ccc3ccccc3c2)cc1-c1ccccc1 ; *N(c1ccc2oc3ccccc3c2c1)c1ccc(-c2ccc3ccccc3c2)cc1-c1ccccc1 ; *N(c1ccc(-c2ccc3ccccc3c2)cc1-c1ccccc1)c1cccc2c1sc1ccccc12]

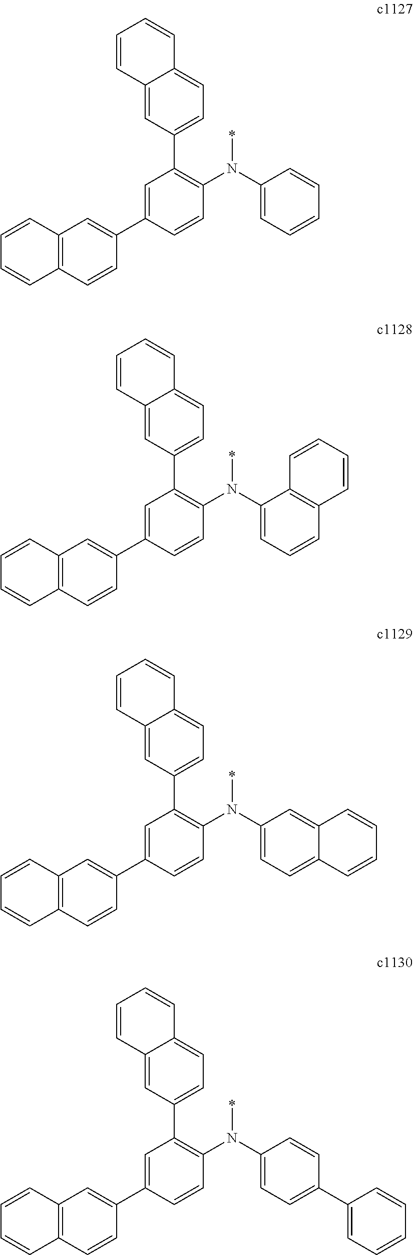 [SMILES: *N(c1ccccc1)c1ccc(-c2ccc3ccccc3c2)cc1-c1ccc2ccccc2c1 ; *N(c1ccc(-c2ccc3ccccc3c2)cc1-c1ccc2ccccc2c1)c1cccc2ccccc12 ; *N(c1ccc2ccccc2c1)c1ccc(-c2ccc3ccccc3c2)cc1-c1ccc2ccccc2c1 ; *N(c1ccc(-c2ccccc2)cc1)c1ccc(-c2ccc3ccccc3c2)cc1-c1ccc2ccccc2c1]

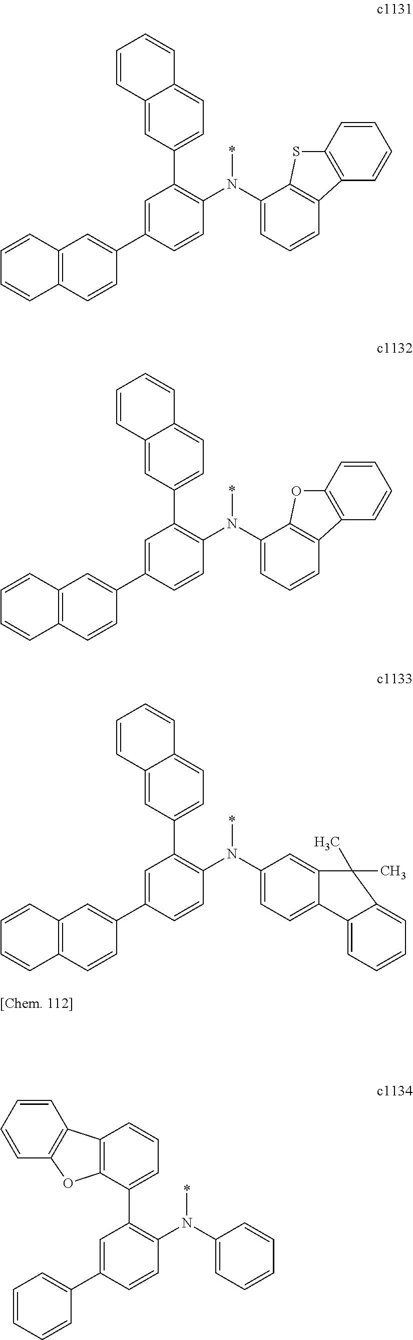 [SMILES: *N(c1ccc(-c2ccc3ccccc3c2)cc1-c1ccc2ccccc2c1)c1cccc2c1sc1ccccc12 ; *N(c1ccc(-c2ccc3ccccc3c2)cc1-c1ccc2ccccc2c1)c1cccc2c1oc1ccccc12 ; *N(c1ccc2c(c1)C(C)(C)c1ccccc1-2)c1ccc(-c2ccc3ccccc3c2)cc1-c1ccc2ccccc2c1 ; *N(c1ccccc1)c1ccc(-c2ccccc2)cc1-c1cccc2c1oc1ccccc12]

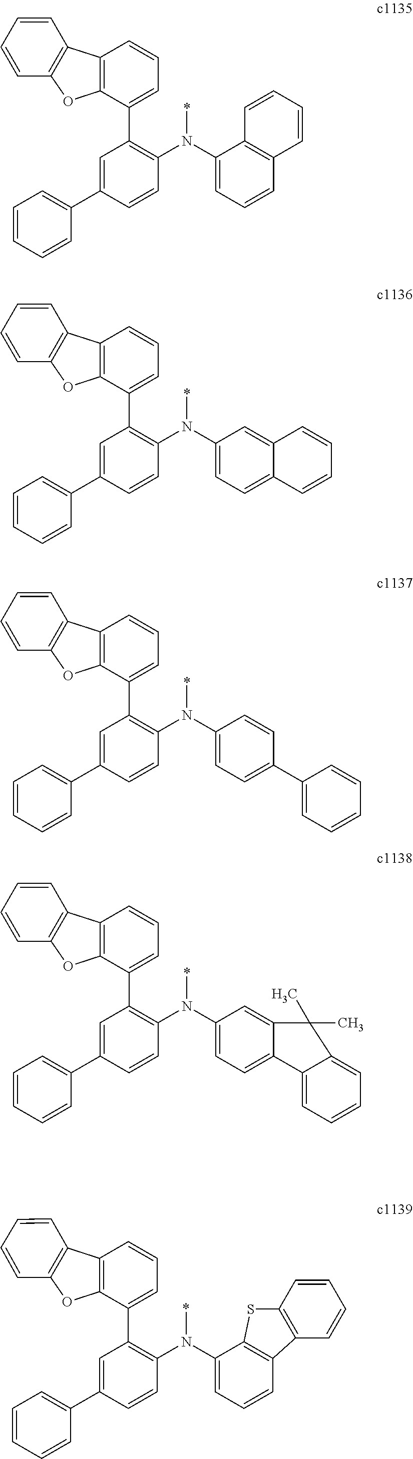 [SMILES: *N(c1ccc(-c2ccccc2)cc1-c1cccc2c1oc1ccccc12)c1cccc2ccccc12 ; *N(c1ccc2ccccc2c1)c1ccc(-c2ccccc2)cc1-c1cccc2c1oc1ccccc12 ; *N(c1ccc(-c2ccccc2)cc1)c1ccc(-c2ccccc2)cc1-c1cccc2c1oc1ccccc12 ; *N(c1ccc2c(c1)C(C)(C)c1ccccc1-2)c1ccc(-c2ccccc2)cc1-c1cccc2c1oc1ccccc12 ; *N(c1ccc(-c2ccccc2)cc1-c1cccc2c1oc1ccccc12)c1cccc2c1sc1ccccc12]

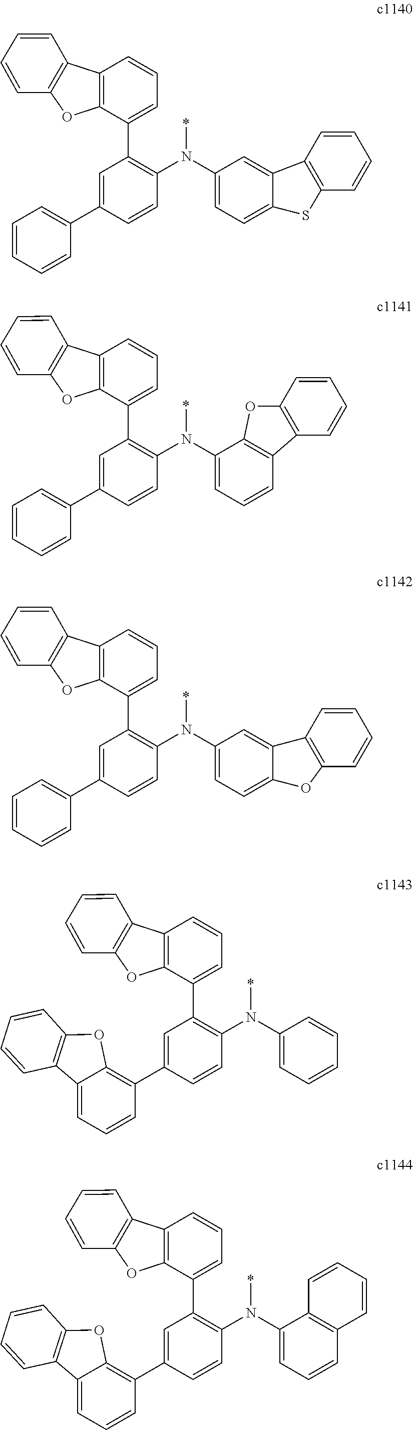

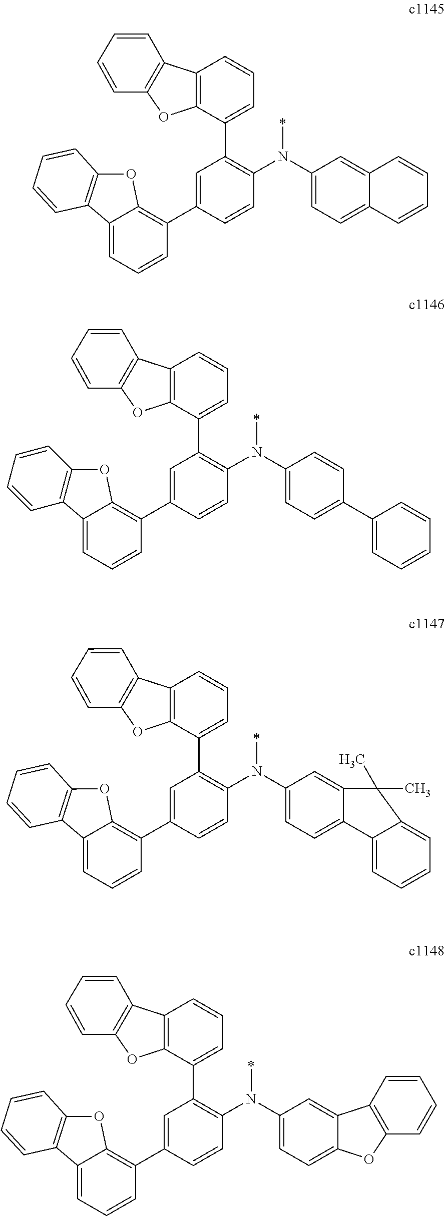 [SMILES: *N(c1ccc2ccccc2c1)c1ccc(-c2cccc3c2oc2ccccc23)cc1-c1cccc2c1oc1ccccc12 ; *N(c1ccc(-c2ccccc2)cc1)c1ccc(-c2cccc3c2oc2ccccc23)cc1-c1cccc2c1oc1ccccc12 ; *N(c1ccc2c(c1)C(C)(C)c1ccccc1-2)c1ccc(-c2cccc3c2oc2ccccc23)cc1-c1cccc2c1oc1ccccc12 ; *N(c1ccc2oc3ccccc3c2c1)c1ccc(-c2cccc3c2oc2ccccc23)cc1-c1cccc2c1oc1ccccc12]

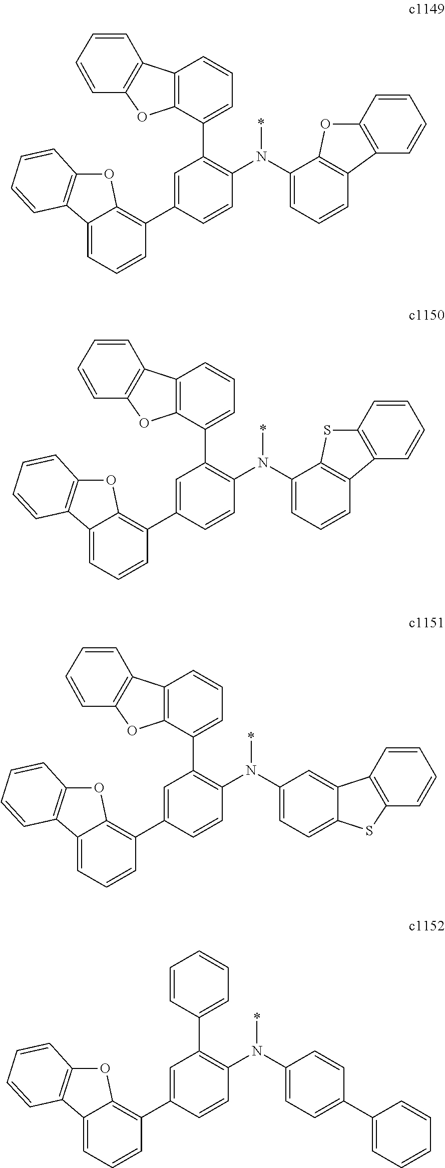 [SMILES: *N(c1ccc(-c2cccc3c2oc2ccccc23)cc1-c1cccc2c1oc1ccccc12)c1cccc2c1oc1ccccc12 ; *N(c1ccc(-c2cccc3c2oc2ccccc23)cc1-c1cccc2c1oc1ccccc12)c1cccc2c1sc1ccccc12 ; *N(c1ccc2sc3ccccc3c2c1)c1ccc(-c2cccc3c2oc2ccccc23)cc1-c1cccc2c1oc1ccccc12 ; *N(c1ccc(-c2ccccc2)cc1)c1ccc(-c2cccc3c2oc2ccccc23)cc1-c1ccccc1]

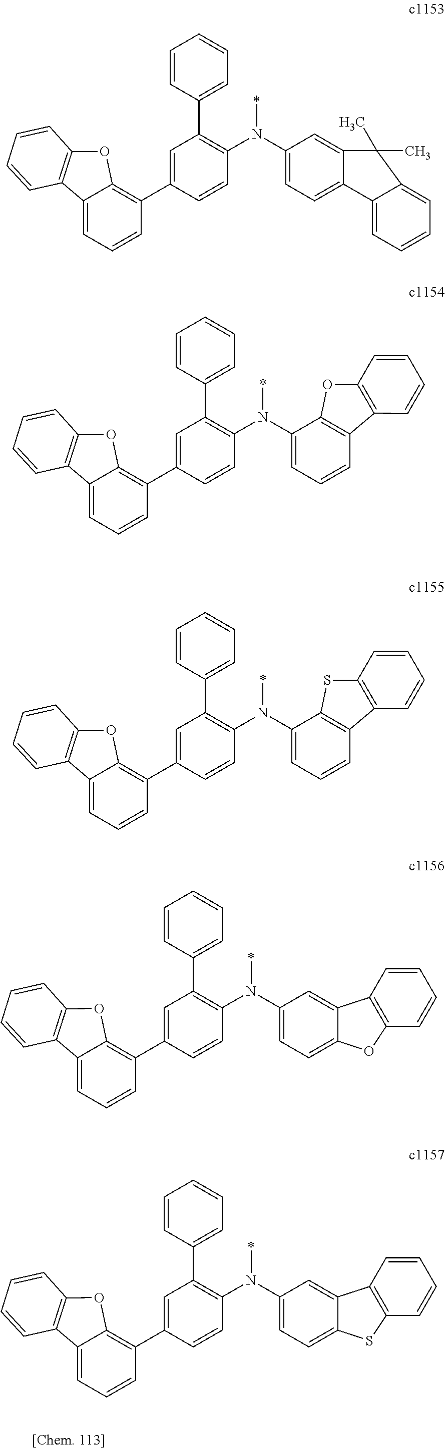 [SMILES: *N(c1ccc2c(c1)C(C)(C)c1ccccc1-2)c1ccc(-c2cccc3c2oc2ccccc23)cc1-c1ccccc1 ; *N(c1ccc(-c2cccc3c2oc2ccccc23)cc1-c1ccccc1)c1cccc2c1oc1ccccc12 ; *N(c1ccc(-c2cccc3c2oc2ccccc23)cc1-c1ccccc1)c1cccc2c1sc1ccccc12 ; *N(c1ccc2oc3ccccc3c2c1)c1ccc(-c2cccc3c2oc2ccccc23)cc1-c1ccccc1 ; *N(c1ccc2sc3ccccc3c2c1)c1ccc(-c2cccc3c2oc2ccccc23)cc1-c1ccccc1]

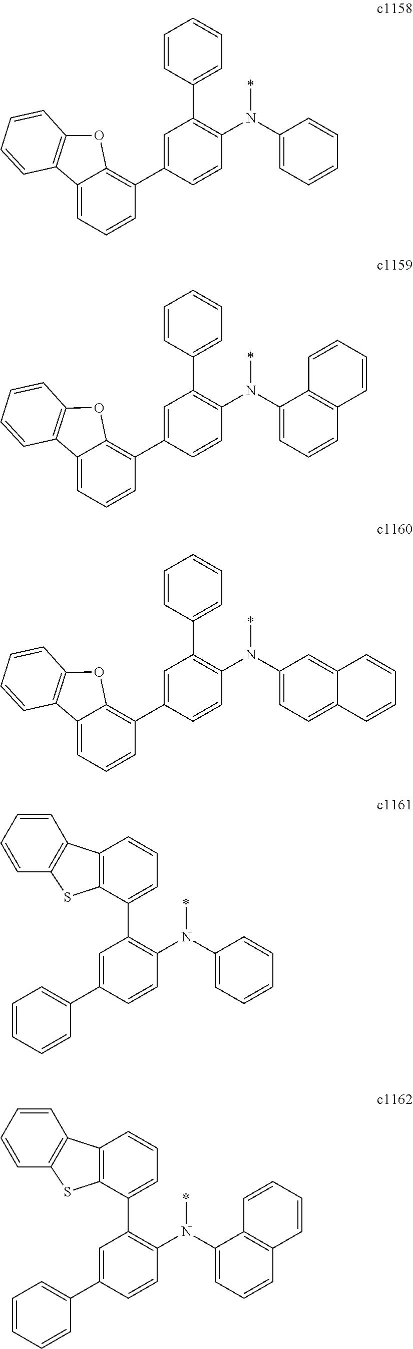 [SMILES: *N(c1ccccc1)c1ccc(-c2cccc3c2oc2ccccc23)cc1-c1ccccc1 ; *N(c1ccc(-c2cccc3c2oc2ccccc23)cc1-c1ccccc1)c1cccc2ccccc12 ; *N(c1ccc2ccccc2c1)c1ccc(-c2cccc3c2oc2ccccc23)cc1-c1ccccc1 ; *N(c1ccccc1)c1ccc(-c2ccccc2)cc1-c1cccc2c1sc1ccccc12 ; *N(c1ccc(-c2ccccc2)cc1-c1cccc2c1sc1ccccc12)c1cccc2ccccc12]

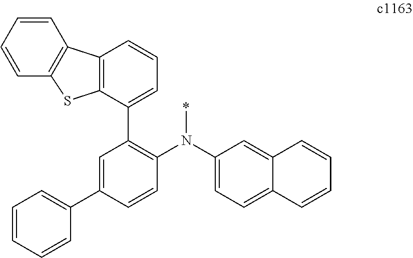 [SMILES: *N(c1ccc2ccccc2c1)c1ccc(-c2ccccc2)cc1-c1cccc2c1sc1ccccc12]

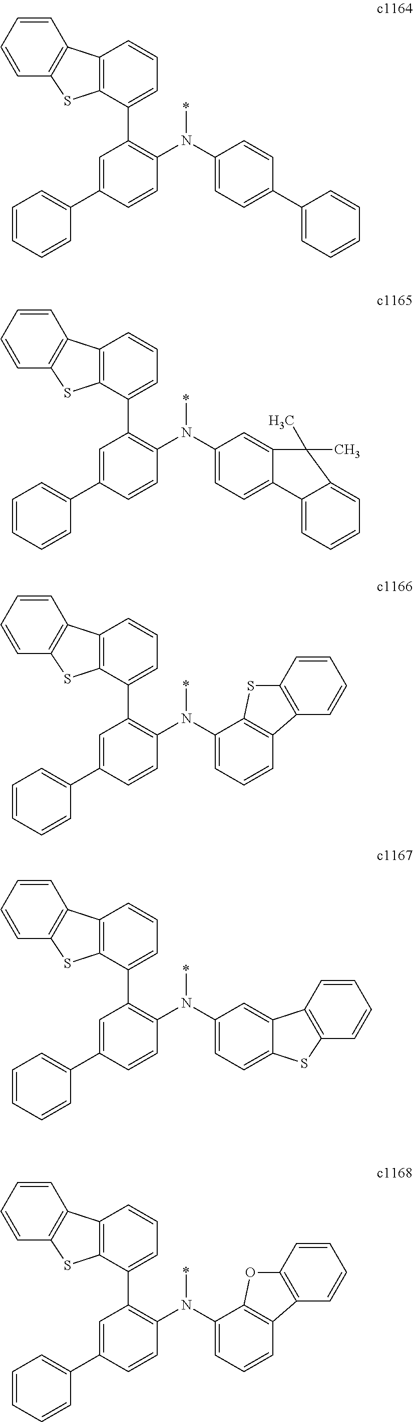

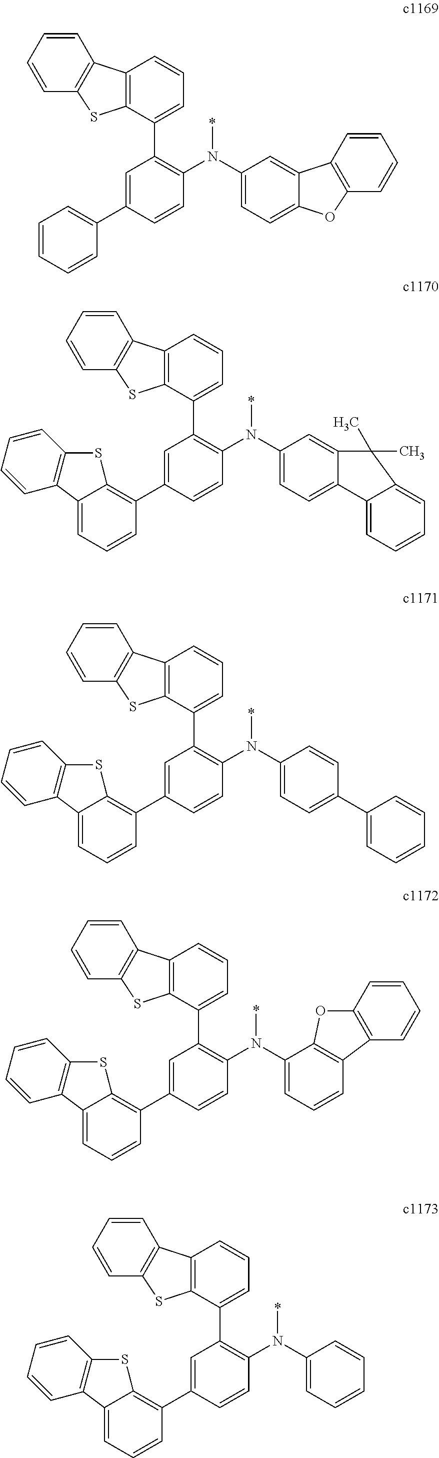

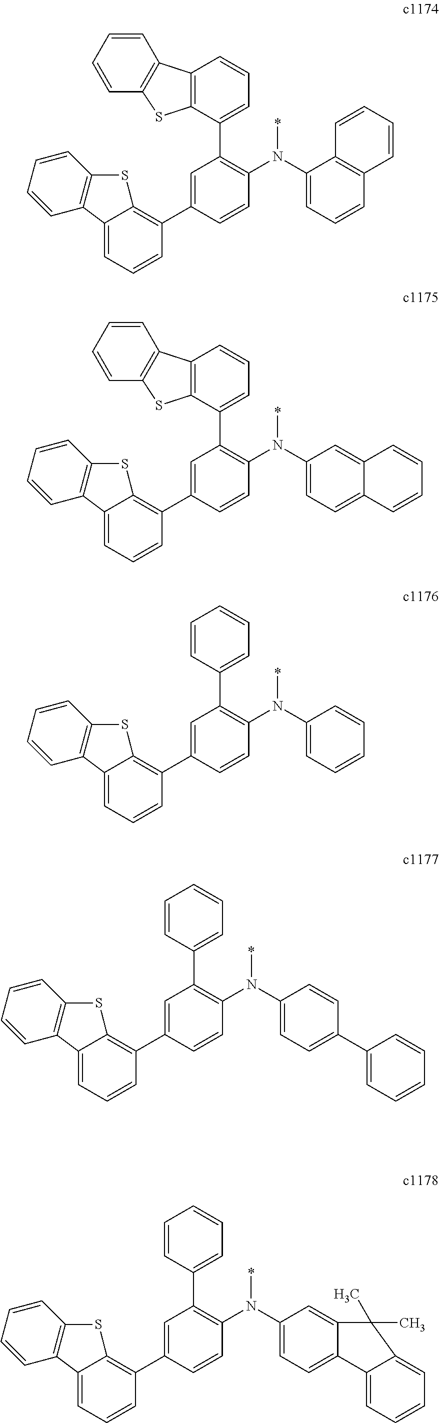 [SMILES: *N(c1ccc(-c2cccc3c2sc2ccccc23)cc1-c1cccc2c1sc1ccccc12)c1cccc2ccccc12 ; *N(c1ccc2ccccc2c1)c1ccc(-c2cccc3c2sc2ccccc23)cc1-c1cccc2c1sc1ccccc12 ; *N(c1ccccc1)c1ccc(-c2cccc3c2sc2ccccc23)cc1-c1ccccc1 ; *N(c1ccc(-c2ccccc2)cc1)c1ccc(-c2cccc3c2sc2ccccc23)cc1-c1ccccc1 ; *N(c1ccc2c(c1)C(C)(C)c1ccccc1-2)c1ccc(-c2cccc3c2sc2ccccc23)cc1-c1ccccc1]

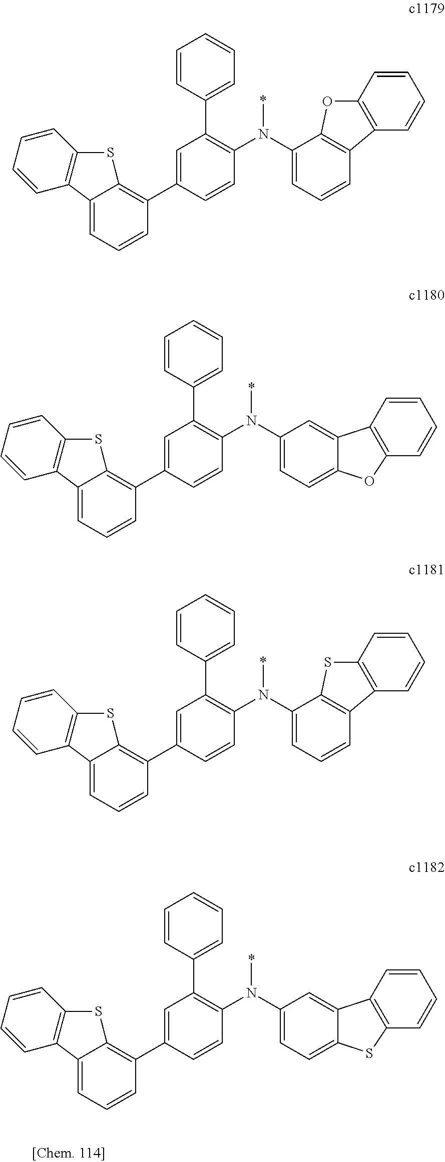 [SMILES: *N(c1ccc(-c2cccc3c2sc2ccccc23)cc1-c1ccccc1)c1cccc2c1oc1ccccc12 ; *N(c1ccc2oc3ccccc3c2c1)c1ccc(-c2cccc3c2sc2ccccc23)cc1-c1ccccc1 ; *N(c1ccc(-c2cccc3c2sc2ccccc23)cc1-c1ccccc1)c1cccc2c1sc1ccccc12 ; *N(c1ccc2sc3ccccc3c2c1)c1ccc(-c2cccc3c2sc2ccccc23)cc1-c1ccccc1]

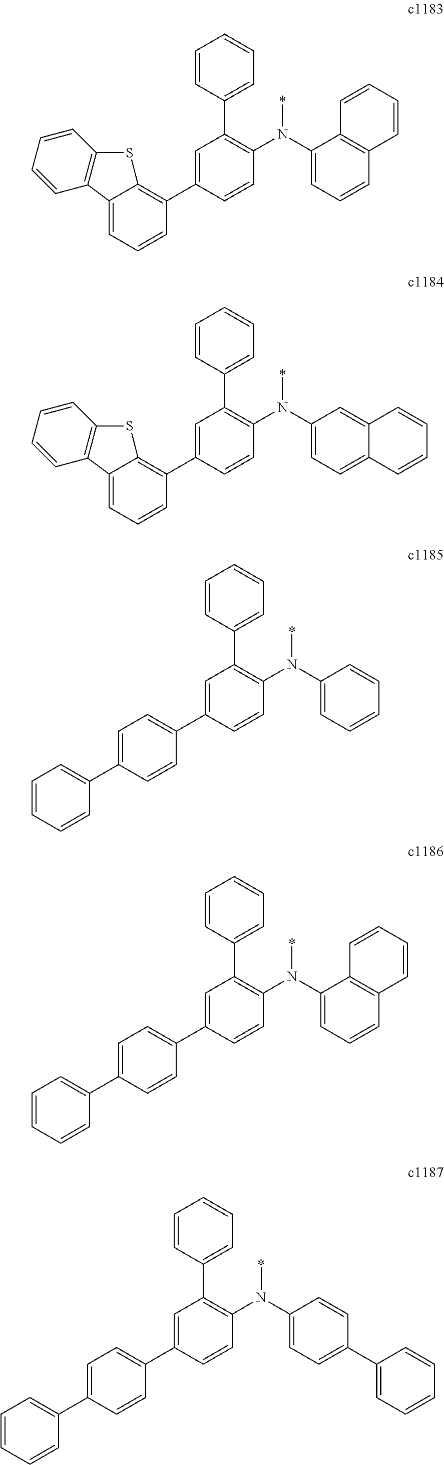 [SMILES: *N(c1ccc(-c2cccc3c2sc2ccccc23)cc1-c1ccccc1)c1cccc2ccccc12 ; *N(c1ccc2ccccc2c1)c1ccc(-c2cccc3c2sc2ccccc23)cc1-c1ccccc1 ; *N(c1ccccc1)c1ccc(-c2ccc(-c3ccccc3)cc2)cc1-c1ccccc1 ; *N(c1ccc(-c2ccc(-c3ccccc3)cc2)cc1-c1ccccc1)c1cccc2ccccc12 ; *N(c1ccc(-c2ccccc2)cc1)c1ccc(-c2ccc(-c3ccccc3)cc2)cc1-c1ccccc1]

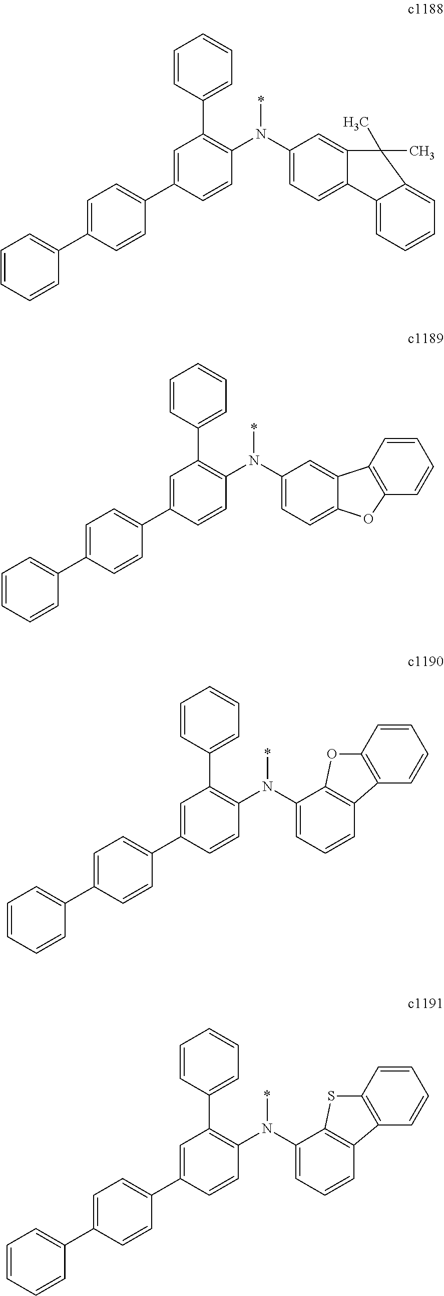 [SMILES: *N(c1ccc2c(c1)C(C)(C)c1ccccc1-2)c1ccc(-c2ccc(-c3ccccc3)cc2)cc1-c1ccccc1 ; *N(c1ccc2oc3ccccc3c2c1)c1ccc(-c2ccc(-c3ccccc3)cc2)cc1-c1ccccc1 ; *N(c1ccc(-c2ccc(-c3ccccc3)cc2)cc1-c1ccccc1)c1cccc2c1oc1ccccc12 ; *N(c1ccc(-c2ccc(-c3ccccc3)cc2)cc1-c1ccccc1)c1cccc2c1sc1ccccc12]

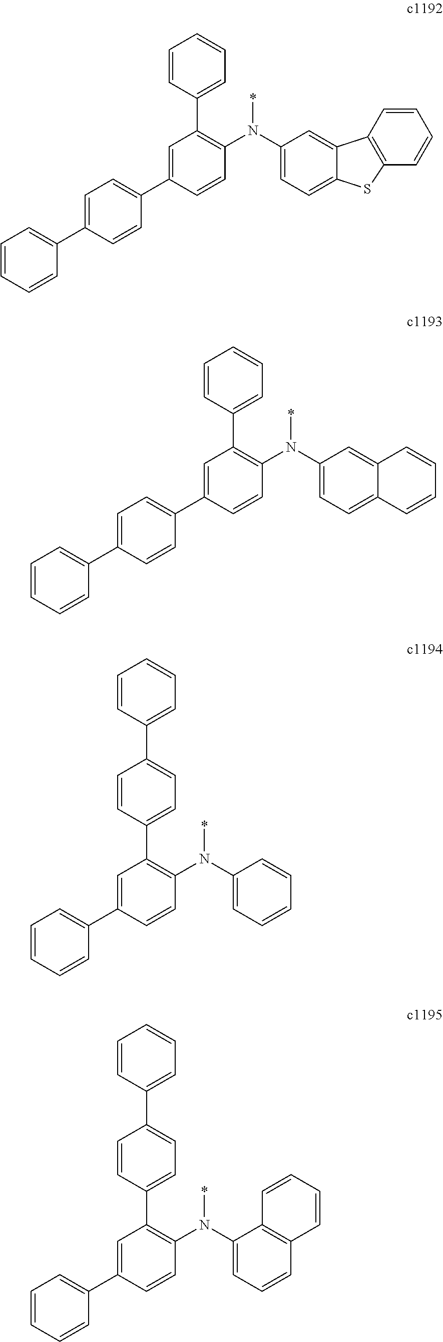 [SMILES: *N(c1ccc2sc3ccccc3c2c1)c1ccc(-c2ccc(-c3ccccc3)cc2)cc1-c1ccccc1 ; *N(c1ccc2ccccc2c1)c1ccc(-c2ccc(-c3ccccc3)cc2)cc1-c1ccccc1 ; *N(c1ccccc1)c1ccc(-c2ccccc2)cc1-c1ccc(-c2ccccc2)cc1 ; *N(c1ccc(-c2ccccc2)cc1-c1ccc(-c2ccccc2)cc1)c1cccc2ccccc12]

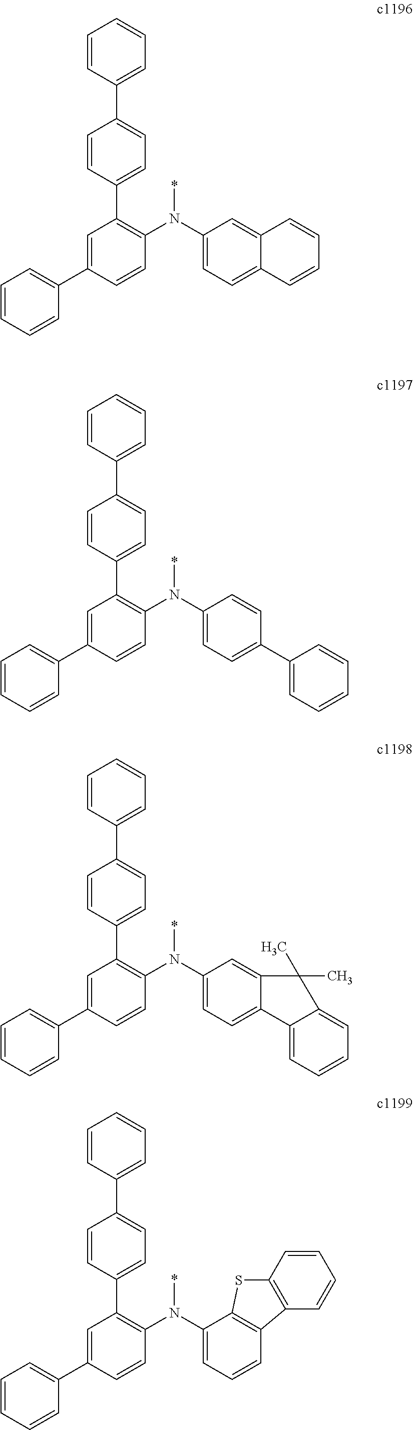 [SMILES: *N(c1ccc2ccccc2c1)c1ccc(-c2ccccc2)cc1-c1ccc(-c2ccccc2)cc1 ; *N(c1ccc(-c2ccccc2)cc1)c1ccc(-c2ccccc2)cc1-c1ccc(-c2ccccc2)cc1 ; *N(c1ccc2c(c1)C(C)(C)c1ccccc1-2)c1ccc(-c2ccccc2)cc1-c1ccc(-c2ccccc2)cc1 ; *N(c1ccc(-c2ccccc2)cc1-c1ccc(-c2ccccc2)cc1)c1cccc2c1sc1ccccc12]

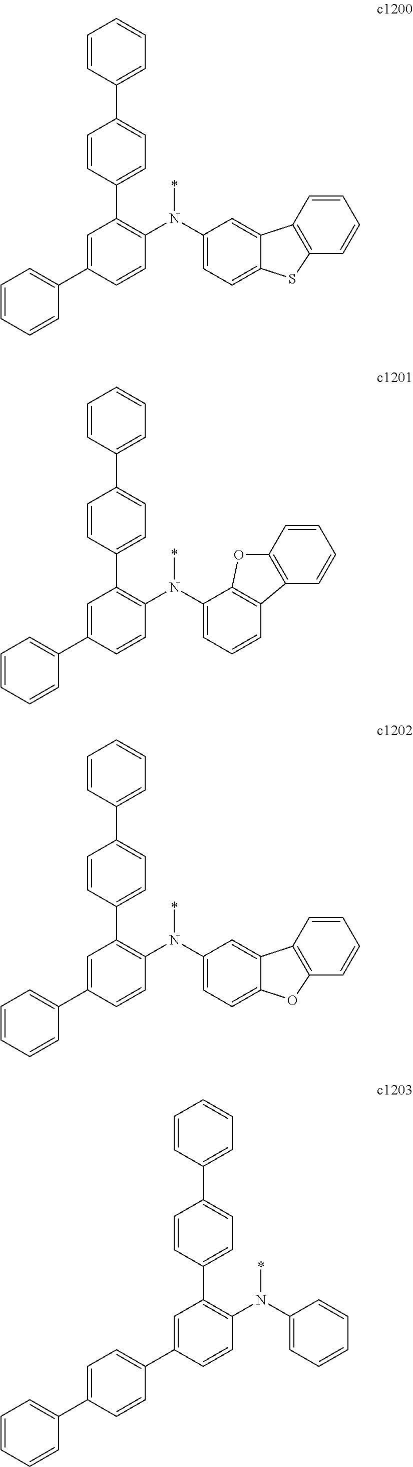 [SMILES: *N(c1ccc2sc3ccccc3c2c1)c1ccc(-c2ccccc2)cc1-c1ccc(-c2ccccc2)cc1 ; *N(c1ccc(-c2ccccc2)cc1-c1ccc(-c2ccccc2)cc1)c1cccc2c1oc1ccccc12 ; *N(c1ccc2oc3ccccc3c2c1)c1ccc(-c2ccccc2)cc1-c1ccc(-c2ccccc2)cc1 ; *N(c1ccccc1)c1ccc(-c2ccc(-c3ccccc3)cc2)cc1-c1ccc(-c2ccccc2)cc1]

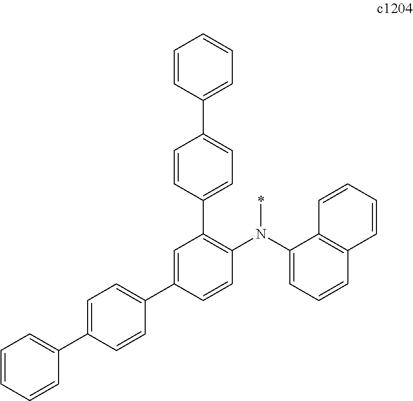 [SMILES: *N(c1ccc(-c2ccc(-c3ccccc3)cc2)cc1-c1ccc(-c2ccccc2)cc1)c1cccc2ccccc12]

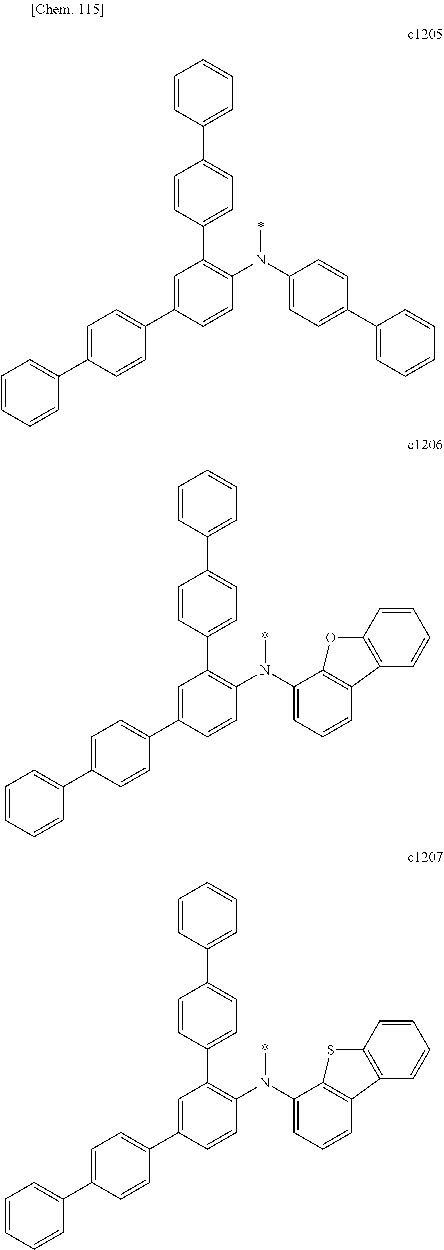 [SMILES: *N(c1ccc(-c2ccccc2)cc1)c1ccc(-c2ccc(-c3ccccc3)cc2)cc1-c1ccc(-c2ccccc2)cc1 ; *N(c1ccc(-c2ccc(-c3ccccc3)cc2)cc1-c1ccc(-c2ccccc2)cc1)c1cccc2c1oc1ccccc12 ; *N(c1ccc(-c2ccc(-c3ccccc3)cc2)cc1-c1ccc(-c2ccccc2)cc1)c1cccc2c1sc1ccccc12]

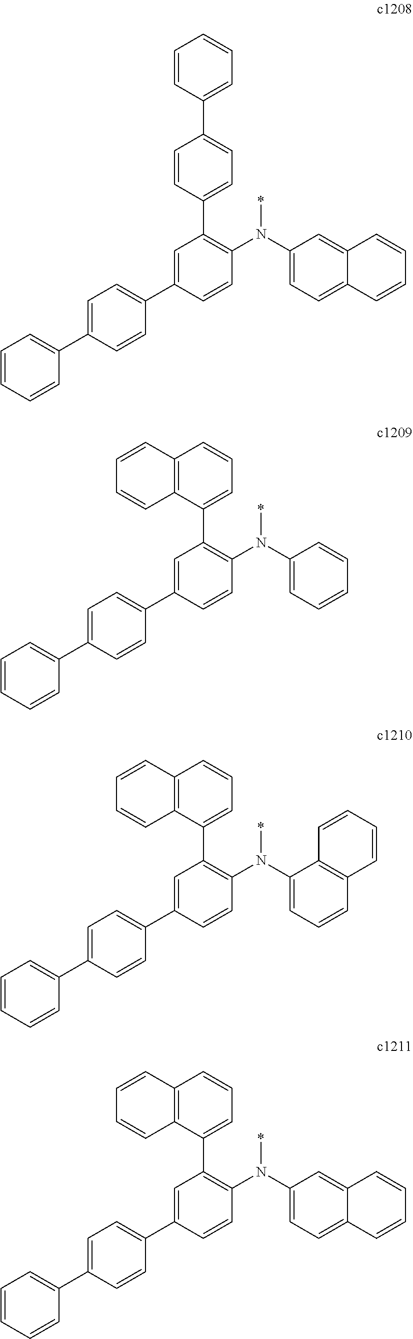 [SMILES: *N(c1ccc2ccccc2c1)c1ccc(-c2ccc(-c3ccccc3)cc2)cc1-c1ccc(-c2ccccc2)cc1 ; *N(c1ccccc1)c1ccc(-c2ccc(-c3ccccc3)cc2)cc1-c1cccc2ccccc12 ; *N(c1ccc(-c2ccc(-c3ccccc3)cc2)cc1-c1cccc2ccccc12)c1cccc2ccccc12 ; *N(c1ccc2ccccc2c1)c1ccc(-c2ccc(-c3ccccc3)cc2)cc1-c1cccc2ccccc12]

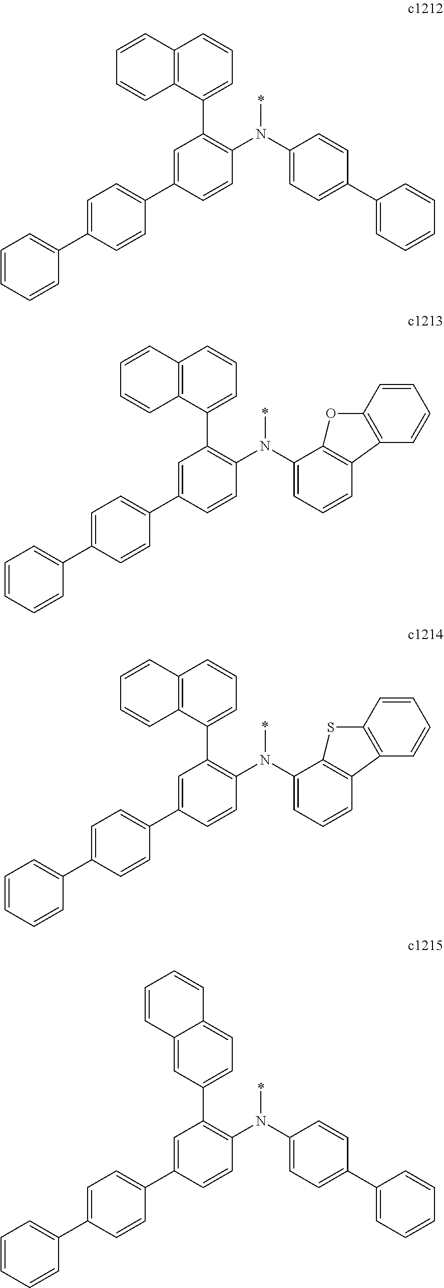 [SMILES: *N(c1ccc(-c2ccccc2)cc1)c1ccc(-c2ccc(-c3ccccc3)cc2)cc1-c1cccc2ccccc12 ; *N(c1ccc(-c2ccc(-c3ccccc3)cc2)cc1-c1cccc2ccccc12)c1cccc2c1oc1ccccc12 ; *N(c1ccc(-c2ccc(-c3ccccc3)cc2)cc1-c1cccc2ccccc12)c1cccc2c1sc1ccccc12 ; *N(c1ccc(-c2ccccc2)cc1)c1ccc(-c2ccc(-c3ccccc3)cc2)cc1-c1ccc2ccccc2c1]

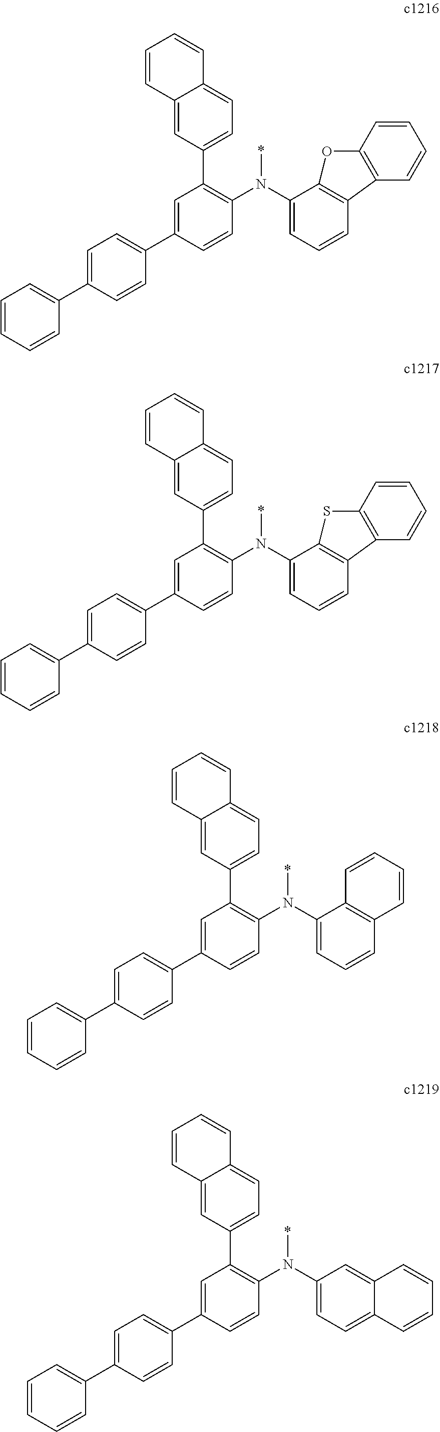 [SMILES: *N(c1ccc(-c2ccc(-c3ccccc3)cc2)cc1-c1ccc2ccccc2c1)c1cccc2c1oc1ccccc12 ; *N(c1ccc(-c2ccc(-c3ccccc3)cc2)cc1-c1ccc2ccccc2c1)c1cccc2c1sc1ccccc12 ; *N(c1ccc(-c2ccc(-c3ccccc3)cc2)cc1-c1ccc2ccccc2c1)c1cccc2ccccc12 ; *N(c1ccc2ccccc2c1)c1ccc(-c2ccc(-c3ccccc3)cc2)cc1-c1ccc2ccccc2c1]

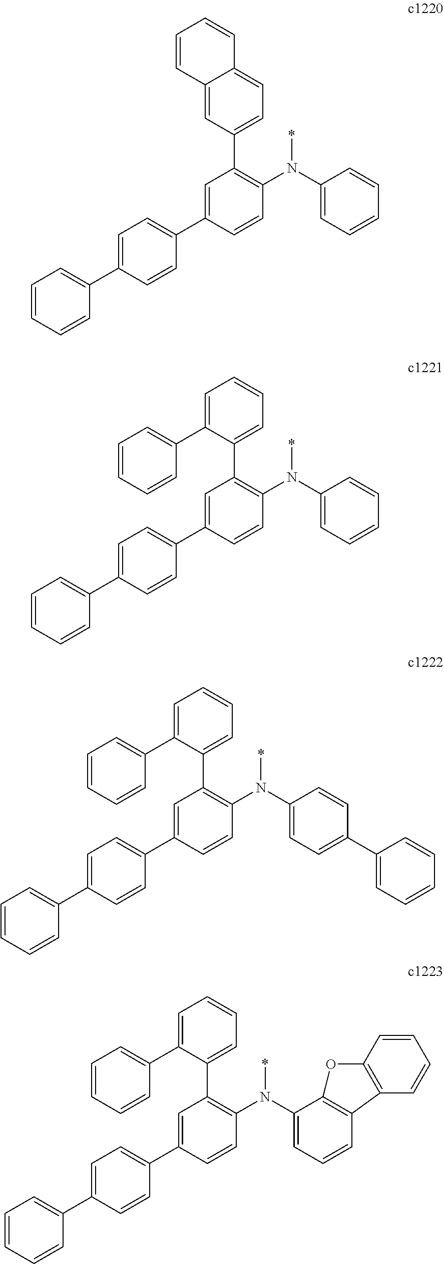 [SMILES: *N(c1ccccc1)c1ccc(-c2ccc(-c3ccccc3)cc2)cc1-c1ccc2ccccc2c1 ; *N(c1ccccc1)c1ccc(-c2ccc(-c3ccccc3)cc2)cc1-c1ccccc1-c1ccccc1 ; *N(c1ccc(-c2ccccc2)cc1)c1ccc(-c2ccc(-c3ccccc3)cc2)cc1-c1ccccc1-c1ccccc1 ; *N(c1ccc(-c2ccc(-c3ccccc3)cc2)cc1-c1ccccc1-c1ccccc1)c1cccc2c1oc1ccccc12]

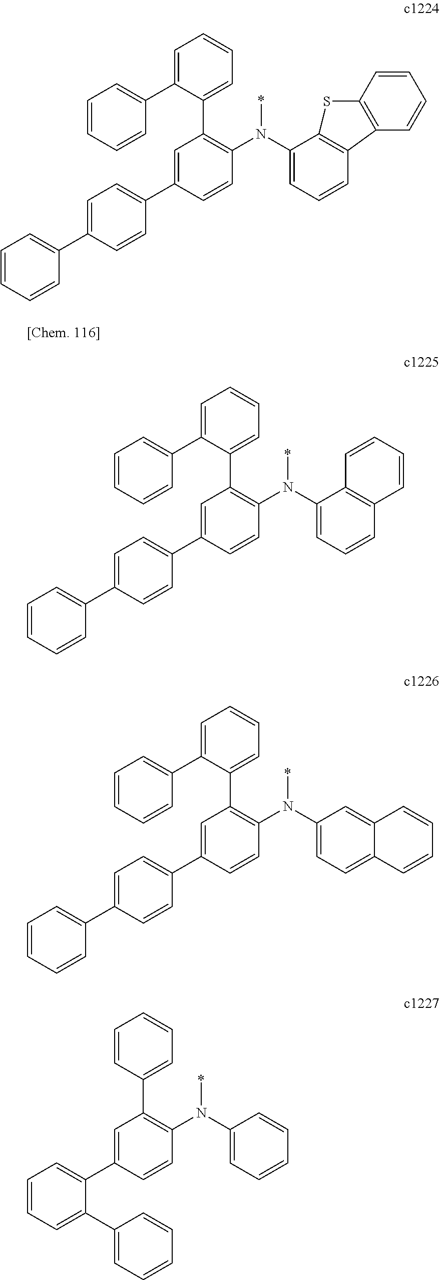 [SMILES: *N(c1ccc(-c2ccc(-c3ccccc3)cc2)cc1-c1ccccc1-c1ccccc1)c1cccc2c1sc1ccccc12 ; *N(c1ccc(-c2ccc(-c3ccccc3)cc2)cc1-c1ccccc1-c1ccccc1)c1cccc2ccccc12 ; *N(c1ccc2ccccc2c1)c1ccc(-c2ccc(-c3ccccc3)cc2)cc1-c1ccccc1-c1ccccc1 ; *N(c1ccccc1)c1ccc(-c2ccccc2-c2ccccc2)cc1-c1ccccc1]

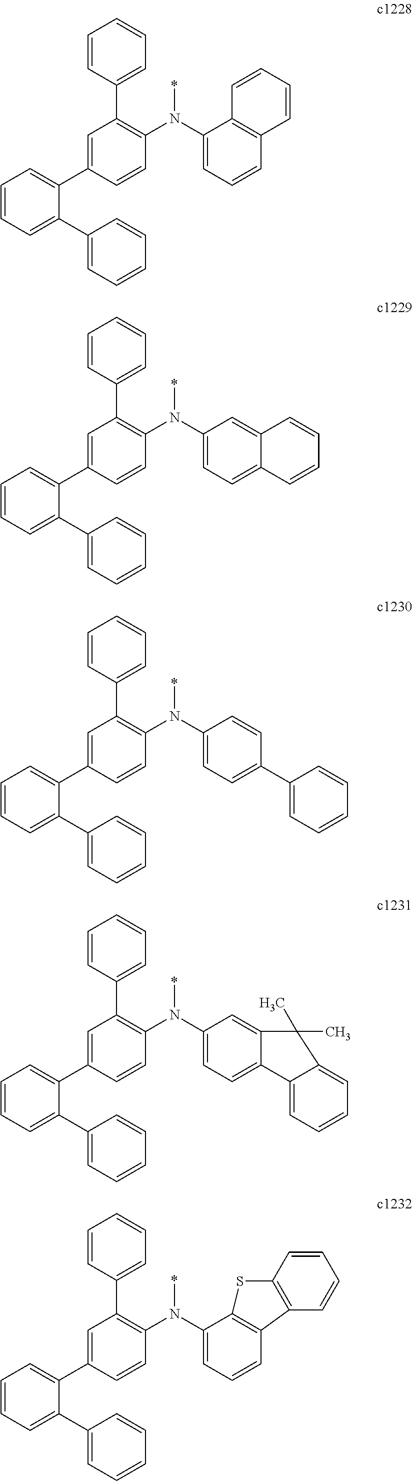

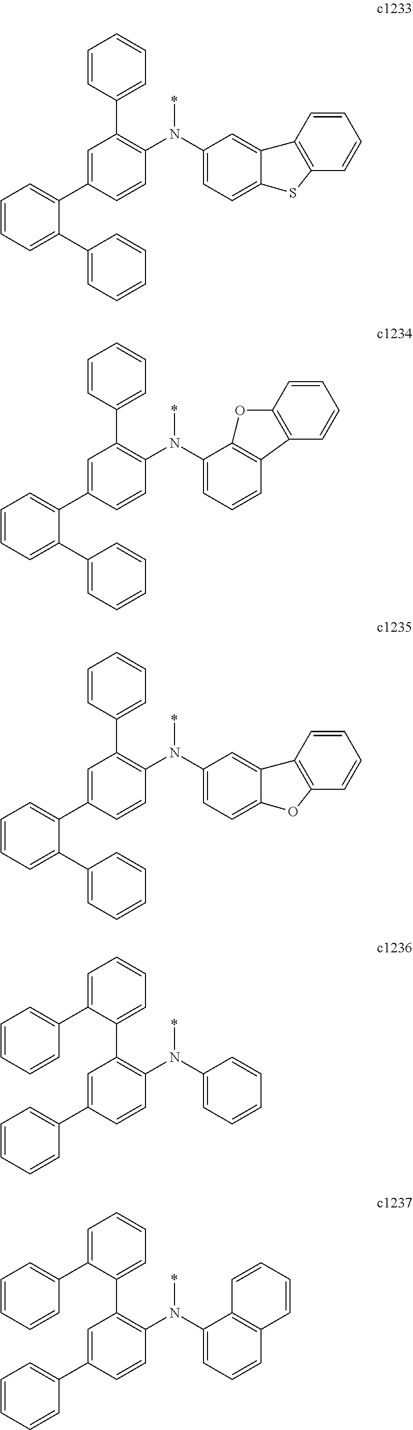

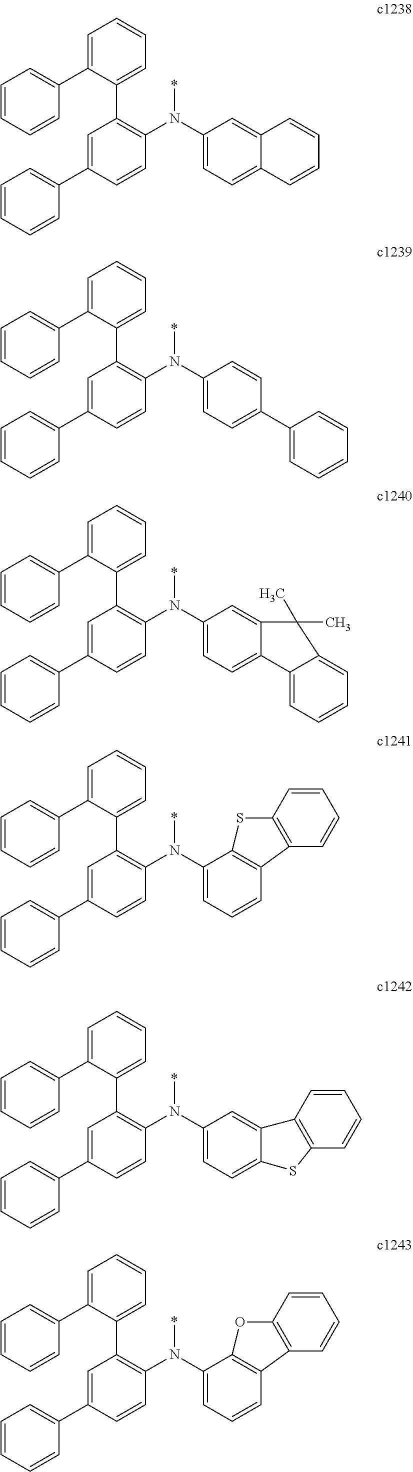 [SMILES: *N(c1ccc2ccccc2c1)c1ccc(-c2ccccc2)cc1-c1ccccc1-c1ccccc1 ; *N(c1ccc(-c2ccccc2)cc1)c1ccc(-c2ccccc2)cc1-c1ccccc1-c1ccccc1 ; *N(c1ccc2c(c1)C(C)(C)c1ccccc1-2)c1ccc(-c2ccccc2)cc1-c1ccccc1-c1ccccc1 ; *N(c1ccc(-c2ccccc2)cc1-c1ccccc1-c1ccccc1)c1cccc2c1sc1ccccc12 ; *N(c1ccc2sc3ccccc3c2c1)c1ccc(-c2ccccc2)cc1-c1ccccc1-c1ccccc1 ; *N(c1ccc(-c2ccccc2)cc1-c1ccccc1-c1ccccc1)c1cccc2c1oc1ccccc12]

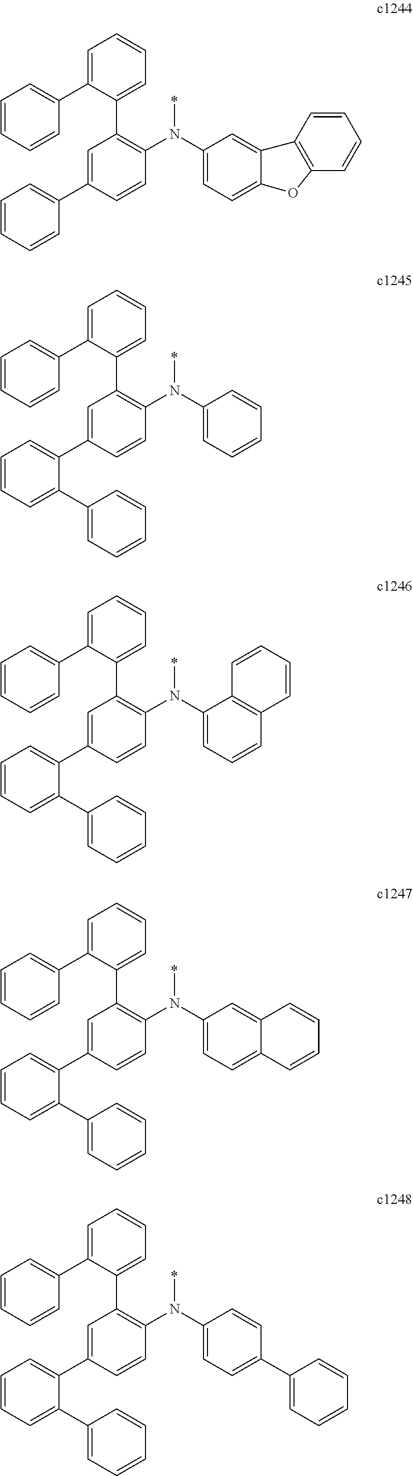

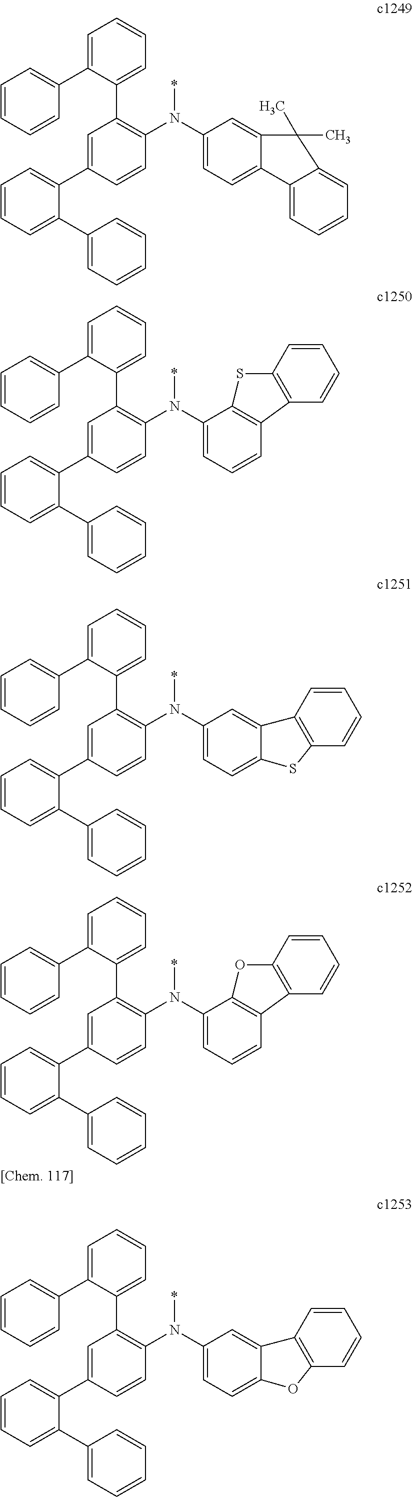

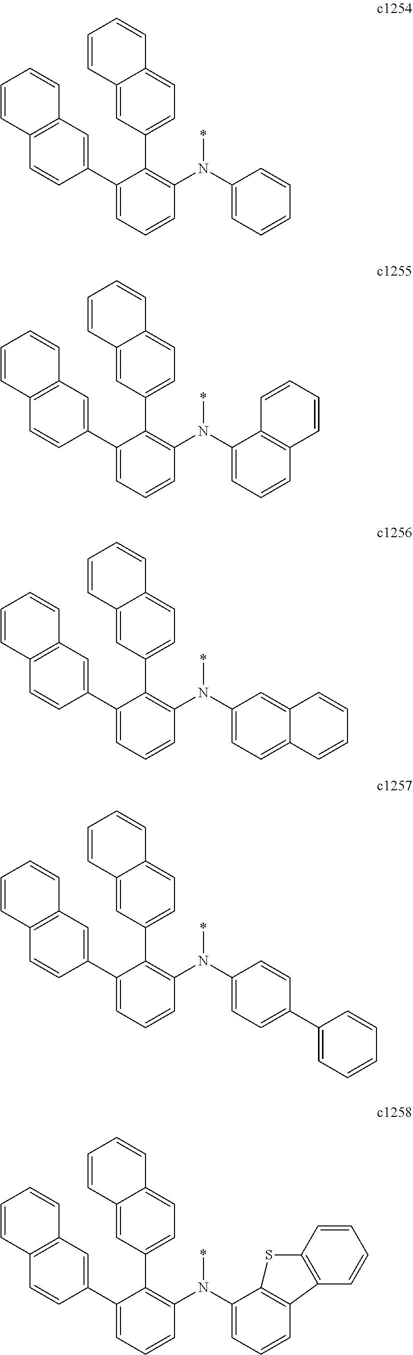 [SMILES: *N(c1ccccc1)c1cccc(-c2ccc3ccccc3c2)c1-c1ccc2ccccc2c1 ; *N(c1cccc(-c2ccc3ccccc3c2)c1-c1ccc2ccccc2c1)c1cccc2ccccc12 ; *N(c1ccc2ccccc2c1)c1cccc(-c2ccc3ccccc3c2)c1-c1ccc2ccccc2c1 ; *N(c1ccc(-c2ccccc2)cc1)c1cccc(-c2ccc3ccccc3c2)c1-c1ccc2ccccc2c1 ; *N(c1cccc(-c2ccc3ccccc3c2)c1-c1ccc2ccccc2c1)c1cccc2c1sc1ccccc12]

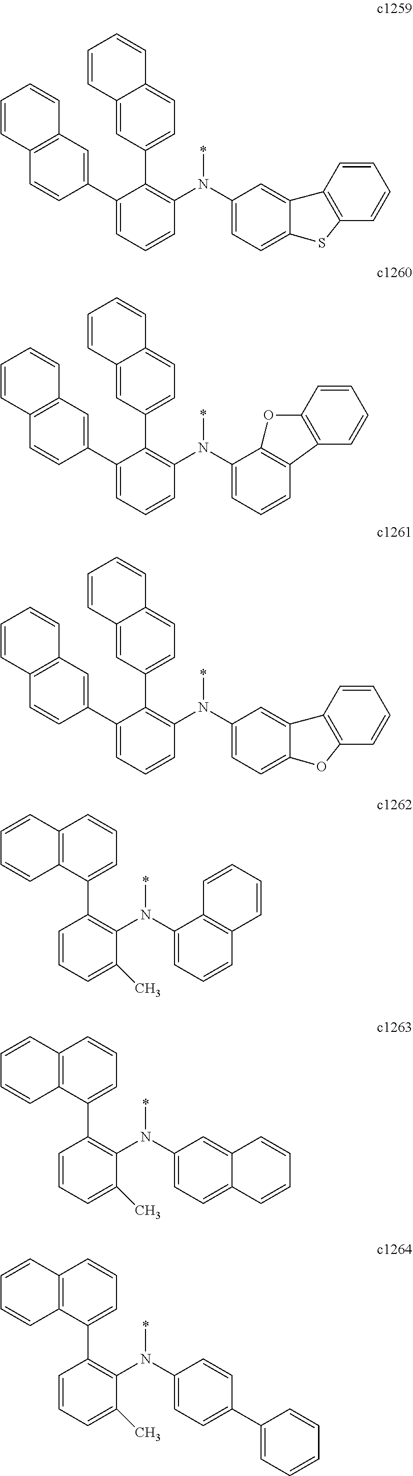 [SMILES: *N(c1ccc2sc3ccccc3c2c1)c1cccc(-c2ccc3ccccc3c2)c1-c1ccc2ccccc2c1 ; *N(c1cccc(-c2ccc3ccccc3c2)c1-c1ccc2ccccc2c1)c1cccc2c1oc1ccccc12 ; *N(c1ccc2oc3ccccc3c2c1)c1cccc(-c2ccc3ccccc3c2)c1-c1ccc2ccccc2c1 ; *N(c1c(C)cccc1-c1cccc2ccccc12)c1cccc2ccccc12 ; *N(c1ccc2ccccc2c1)c1c(C)cccc1-c1cccc2ccccc12 ; *N(c1ccc(-c2ccccc2)cc1)c1c(C)cccc1-c1cccc2ccccc12]

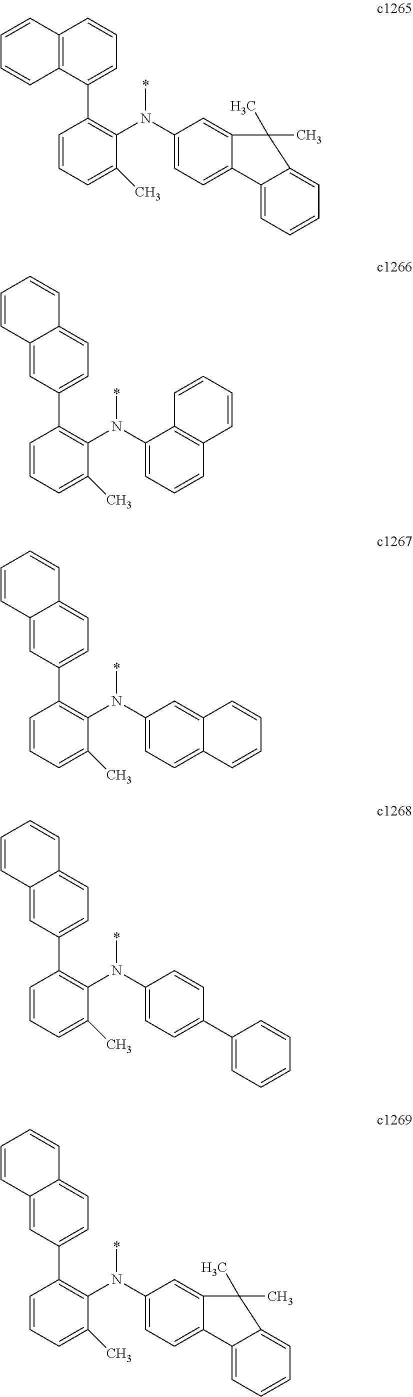 [SMILES: *N(c1ccc2c(c1)C(C)(C)c1ccccc1-2)c1c(C)cccc1-c1cccc2ccccc12 ; *N(c1c(C)cccc1-c1ccc2ccccc2c1)c1cccc2ccccc12 ; *N(c1ccc2ccccc2c1)c1c(C)cccc1-c1ccc2ccccc2c1 ; *N(c1ccc(-c2ccccc2)cc1)c1c(C)cccc1-c1ccc2ccccc2c1 ; *N(c1ccc2c(c1)C(C)(C)c1ccccc1-2)c1c(C)cccc1-c1ccc2ccccc2c1]

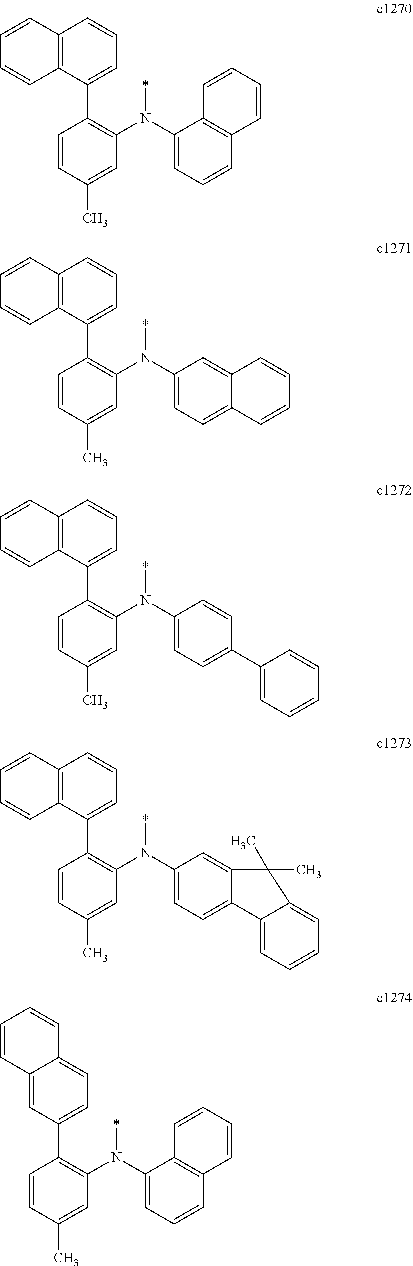

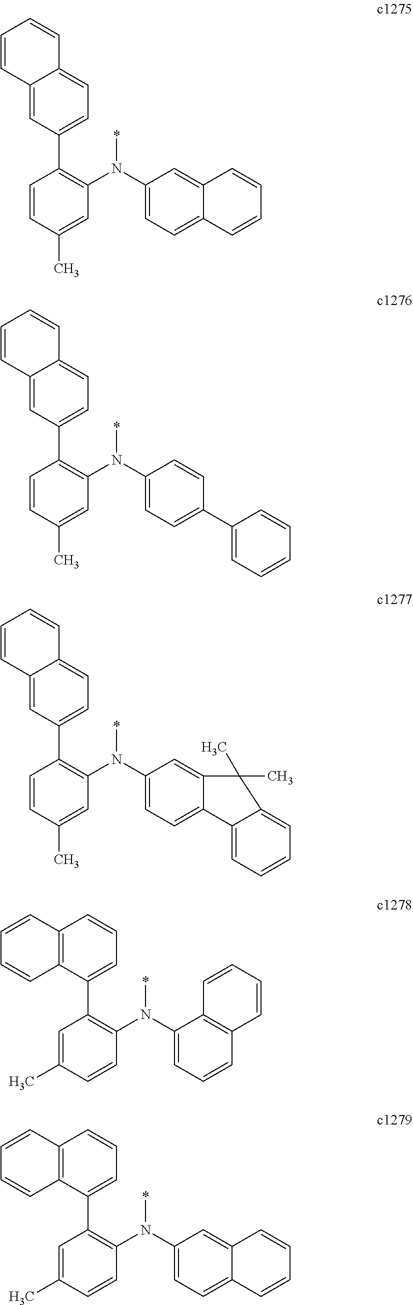 [SMILES: *N(c1ccc2ccccc2c1)c1cc(C)ccc1-c1ccc2ccccc2c1 ; *N(c1ccc(-c2ccccc2)cc1)c1cc(C)ccc1-c1ccc2ccccc2c1 ; *N(c1ccc2c(c1)C(C)(C)c1ccccc1-2)c1cc(C)ccc1-c1ccc2ccccc2c1 ; *N(c1ccc(C)cc1-c1cccc2ccccc12)c1cccc2ccccc12 ; *N(c1ccc2ccccc2c1)c1ccc(C)cc1-c1cccc2ccccc12]

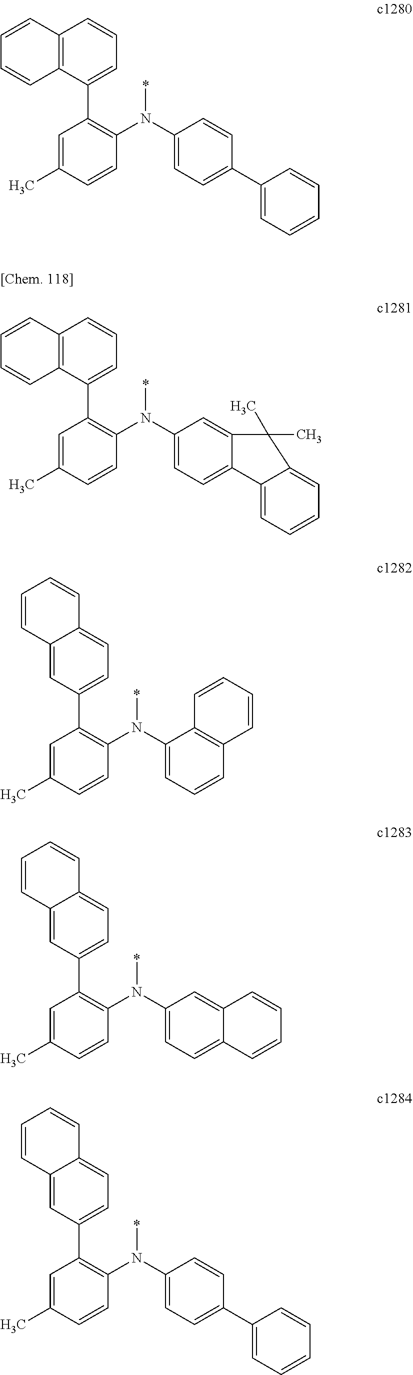 [SMILES: *N(c1ccc(-c2ccccc2)cc1)c1ccc(C)cc1-c1cccc2ccccc12 ; *N(c1ccc2c(c1)C(C)(C)c1ccccc1-2)c1ccc(C)cc1-c1cccc2ccccc12 ; *N(c1ccc(C)cc1-c1ccc2ccccc2c1)c1cccc2ccccc12 ; *N(c1ccc2ccccc2c1)c1ccc(C)cc1-c1ccc2ccccc2c1 ; *N(c1ccc(-c2ccccc2)cc1)c1ccc(C)cc1-c1ccc2ccccc2c1]

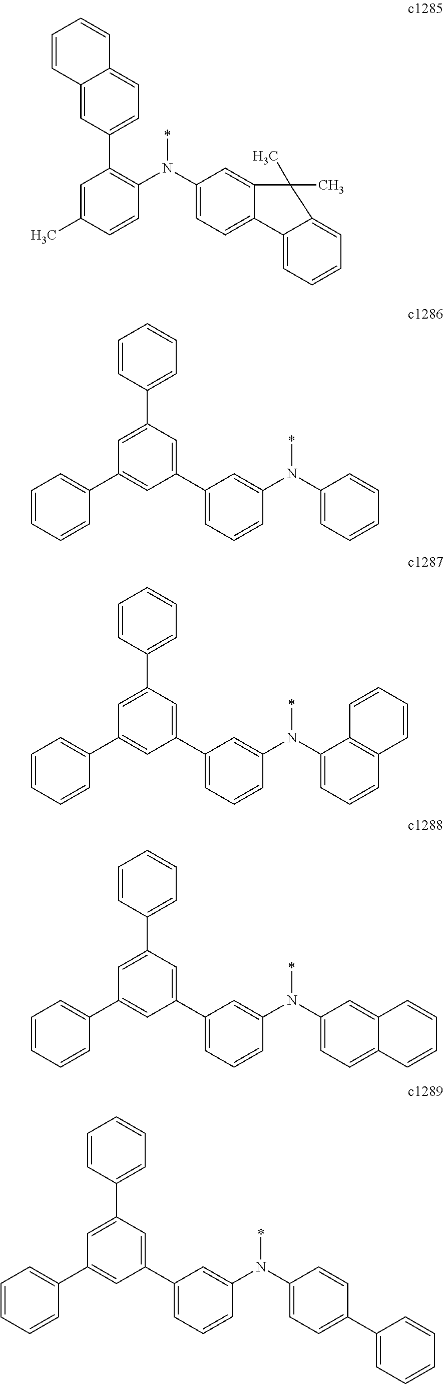 [SMILES: *N(c1ccc2c(c1)C(C)(C)c1ccccc1-2)c1ccc(C)cc1-c1ccc2ccccc2c1 ; *N(c1ccccc1)c1cccc(-c2cc(-c3ccccc3)cc(-c3ccccc3)c2)c1 ; *N(c1cccc(-c2cc(-c3ccccc3)cc(-c3ccccc3)c2)c1)c1cccc2ccccc12 ; *N(c1cccc(-c2cc(-c3ccccc3)cc(-c3ccccc3)c2)c1)c1ccc2ccccc2c1 ; *N(c1ccc(-c2ccccc2)cc1)c1cccc(-c2cc(-c3ccccc3)cc(-c3ccccc3)c2)c1]

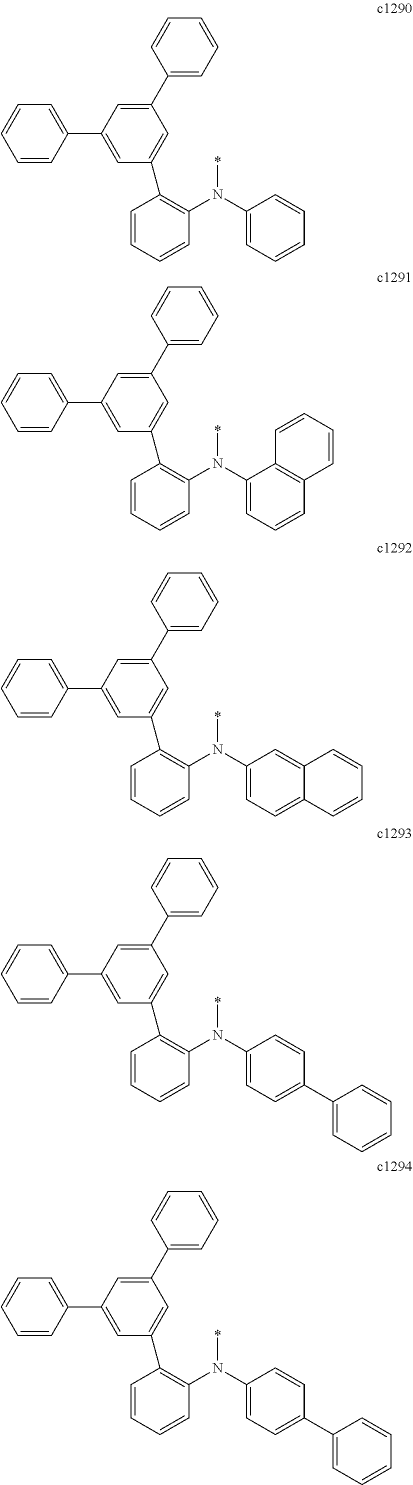

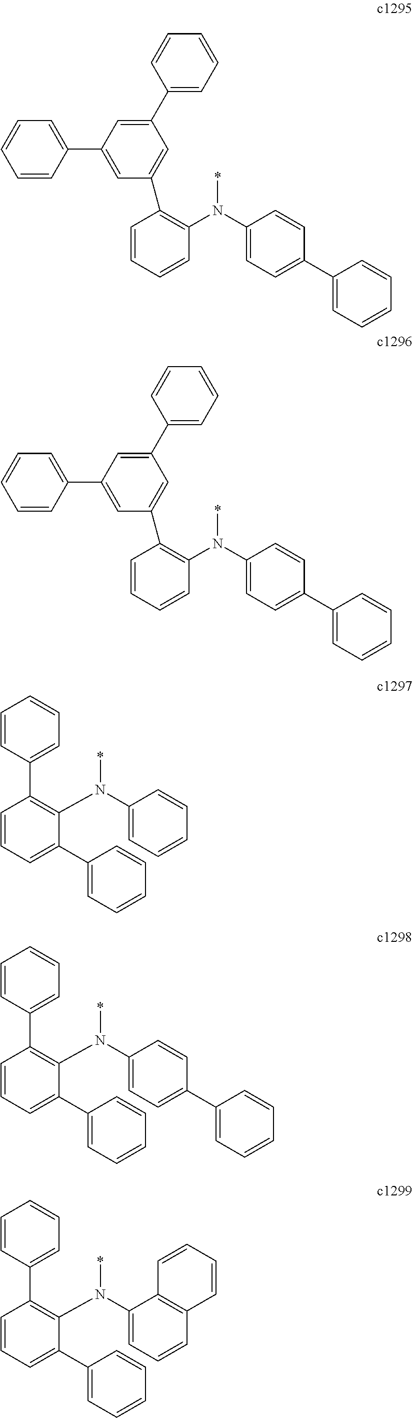 [SMILES: *N(c1ccc(-c2ccccc2)cc1)c1ccccc1-c1cc(-c2ccccc2)cc(-c2ccccc2)c1 ; *N(c1ccc(-c2ccccc2)cc1)c1ccccc1-c1cc(-c2ccccc2)cc(-c2ccccc2)c1 ; *N(c1ccccc1)c1c(-c2ccccc2)cccc1-c1ccccc1 ; *N(c1ccc(-c2ccccc2)cc1)c1c(-c2ccccc2)cccc1-c1ccccc1 ; *N(c1c(-c2ccccc2)cccc1-c1ccccc1)c1cccc2ccccc12]

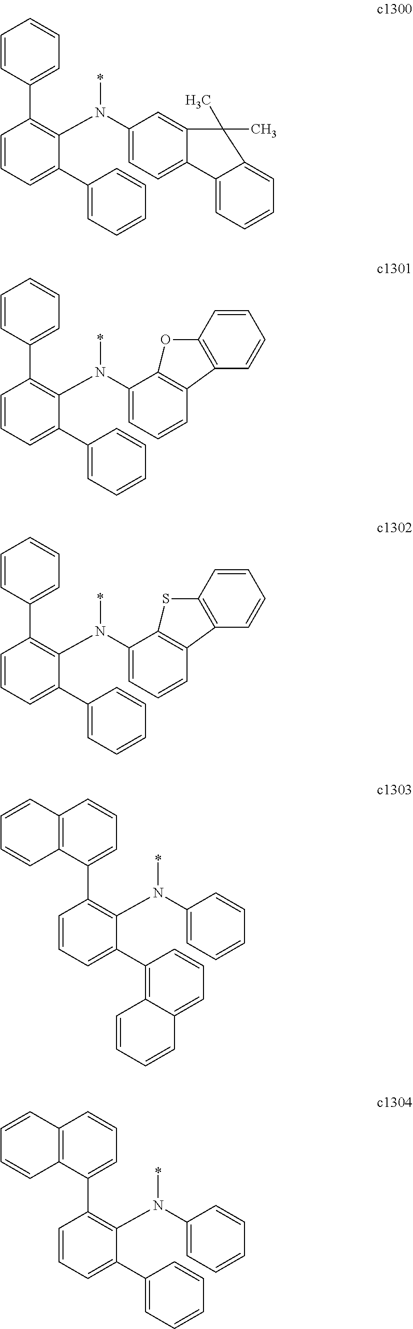 [SMILES: *N(c1ccc2c(c1)C(C)(C)c1ccccc1-2)c1c(-c2ccccc2)cccc1-c1ccccc1 ; *N(c1c(-c2ccccc2)cccc1-c1ccccc1)c1cccc2c1oc1ccccc12 ; *N(c1c(-c2ccccc2)cccc1-c1ccccc1)c1cccc2c1sc1ccccc12 ; *N(c1ccccc1)c1c(-c2cccc3ccccc23)cccc1-c1cccc2ccccc12 ; *N(c1ccccc1)c1c(-c2ccccc2)cccc1-c1cccc2ccccc12]

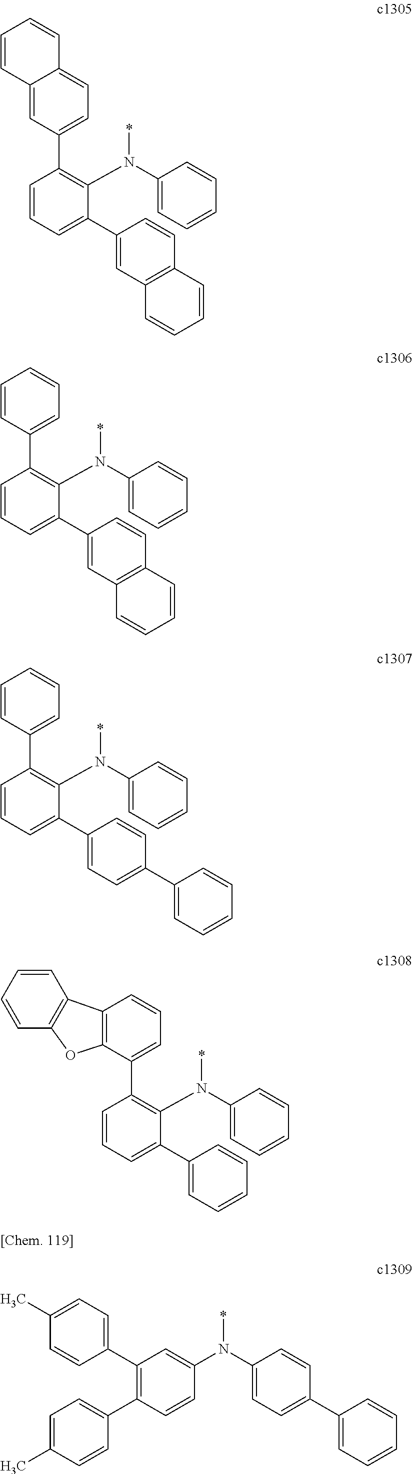 [SMILES: *N(c1ccccc1)c1c(-c2ccc3ccccc3c2)cccc1-c1ccc2ccccc2c1 ; *N(c1ccccc1)c1c(-c2ccccc2)cccc1-c1ccc2ccccc2c1 ; *N(c1ccccc1)c1c(-c2ccccc2)cccc1-c1ccc(-c2ccccc2)cc1 ; *N(c1ccccc1)c1c(-c2ccccc2)cccc1-c1cccc2c1oc1ccccc12 ; *N(c1ccc(-c2ccccc2)cc1)c1ccc(-c2ccc(C)cc2)c(-c2ccc(C)cc2)c1]

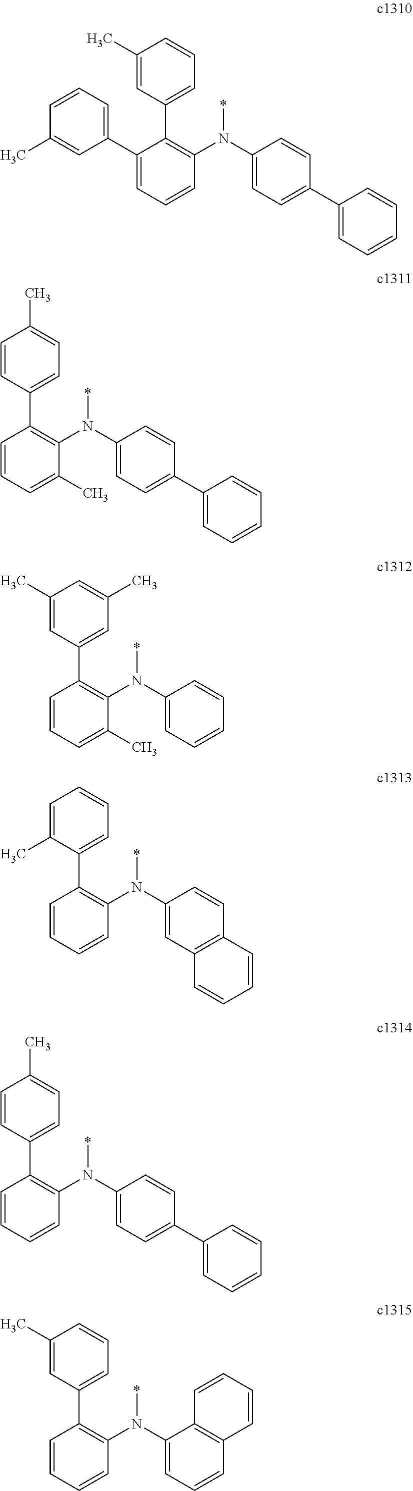

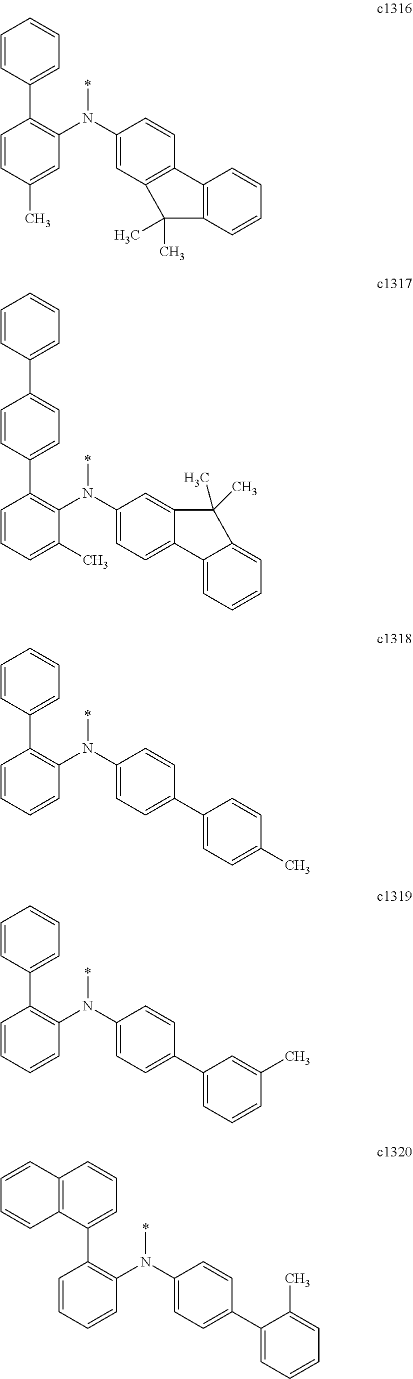 [SMILES: *N(c1ccc2c(c1)C(C)(C)c1ccccc1-2)c1cc(C)ccc1-c1ccccc1 ; *N(c1ccc2c(c1)C(C)(C)c1ccccc1-2)c1c(C)cccc1-c1ccc(-c2ccccc2)cc1 ; *N(c1ccc(-c2ccc(C)cc2)cc1)c1ccccc1-c1ccccc1 ; *N(c1ccc(-c2cccc(C)c2)cc1)c1ccccc1-c1ccccc1 ; *N(c1ccc(-c2ccccc2C)cc1)c1ccccc1-c1cccc2ccccc12]

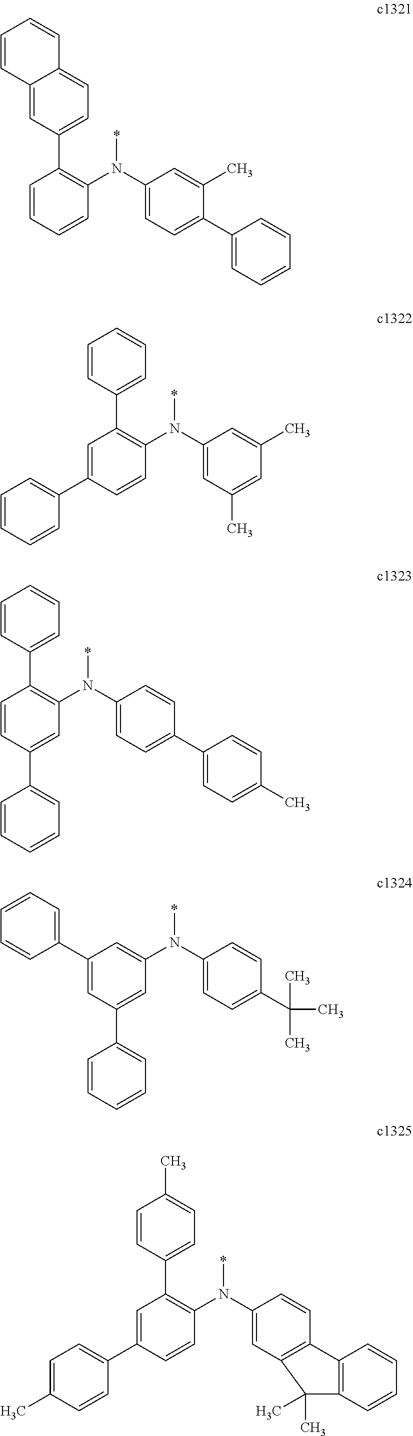

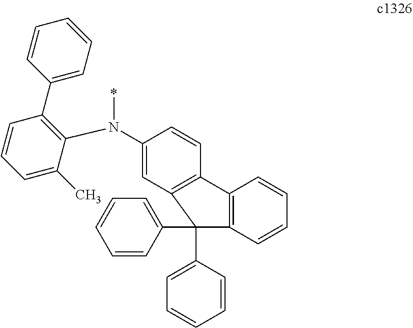 [SMILES: *N(c1ccc2c(c1)C(c1ccccc1)(c1ccccc1)c1ccccc1-2)c1c(C)cccc1-c1ccccc1]

### [Carbazole Compound]

A carbazole compound according to an embodiment of the present disclosure is represented by the formula (22) or (23):

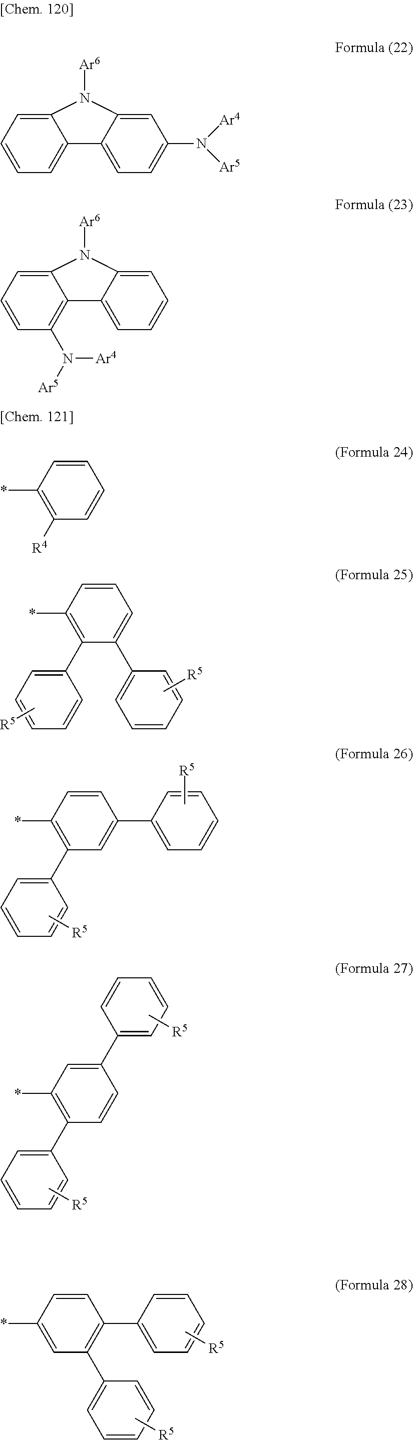

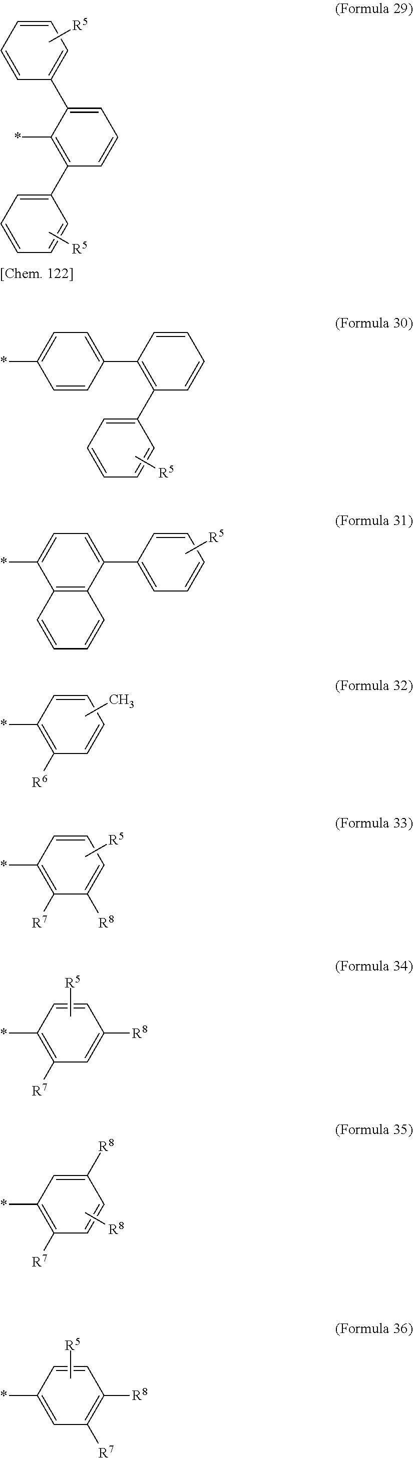

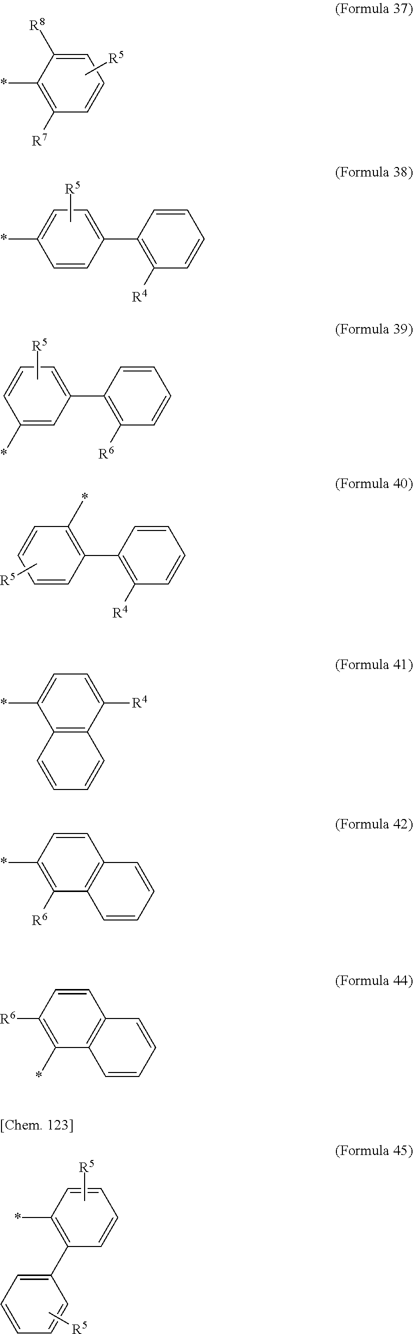

wherein
Ar⁶ each independently denotes a group selected from the formulae (24) to (45).

In the formulae,
R⁴ denotes a biphenylyl group, a naphthyl group, a phenanthryl group, a dibenzofuranyl group or a dibenzothienyl group each optionally substituted with a methyl group.
R⁵ each independently denotes a methyl group or a hydrogen atom.
R⁶ denotes a phenyl group, a biphenylyl group, a naphthyl group, a phenanthryl group, a dibenzofuranyl group or a dibenzothienyl group each optionally substituted with a methyl group.
R⁷ and R⁸ each independently denote a phenyl group, a biphenylyl group, a naphthyl group, a phenanthryl group, a dibenzofuranyl group or a dibenzothienyl group each optionally substituted with a methyl group, and at least one of R⁷ and R⁶ denotes a biphenylyl group, a naphthyl group, a phenanthryl group, a dibenzofuranyl group or a dibenzothienyl group each optionally substituted with a methyl group.

In the formulae (22) and (23), when Ar⁶ denotes a group selected from the formulae (24) to (31),
Ar⁵ denotes a group selected from the formulae (24) to (45), and Ar⁴ denotes an optionally substituted monocyclic, linked or fused aromatic hydrocarbon group with 6 to 30 carbon atoms or a group represented by an optionally substituted monocyclic, linked or fused heteroaromatic group with 3 to 30 carbon atoms.

In the formulae (22) and (23), when Ar⁶ denotes a group selected from the formulae (32) to (44),
Ar⁴ and Ar⁵ each independently denote a group selected from the formulae (24) to (45), an optionally substituted monocyclic, linked or fused aromatic hydrocarbon group with 6 to 30 carbon atoms or a group represented by an optionally substituted monocyclic, linked or fused heteroaromatic group with 3 to 30 carbon atoms.

The optionally substituted monocyclic, linked or fused aromatic hydrocarbon group with 6 to 30 carbon atoms; and the group represented by an optionally substituted monocyclic, linked or fused heteroaromatic group with 3 to 30 carbon atoms in the formulae (22) and (23) are synonymous with the optionally substituted monocyclic, linked or fused aromatic hydrocarbon group with 6 to 30 carbon atoms; and the group represented by an optionally substituted monocyclic, linked or fused heteroaromatic group with 3 to 30 carbon atoms in the formula (1).

Due to good hole-transport properties, the optionally substituted monocyclic, linked or fused aromatic hydrocarbon group with 6 to 30 carbon atoms or the optionally substituted monocyclic, linked or fused heteroaromatic group with 3 to 30 carbon atoms in Ar⁴ of the formulae (22) and (23) preferably each independently denotes
(i) a phenyl group, a biphenylyl group, a terphenylyl group, a naphthyl group, a fluorenyl group, a spirobifluorenyl group, a benzofluorenyl group, a phenanthryl group, a fluoranthenyl group, a triphenylenyl group, an anthryl group, a pyrenyl group, a benzofuranyl group, a benzothienyl group, a dibenzofuranyl group or a dibenzothienyl group or
(ii) the group represented by (i) substituted with one or more groups selected from the group consisting of a methyl group, an ethyl group, a methoxy group, an ethoxy group, a cyano group, a deuterium atom, a fluorine atom, a phenyl group, a biphenylyl group, a naphthyl group, a phenanthryl group, a triphenylsilyl group, a carbazolyl group, a dibenzothienyl group and a dibenzofuranyl group.

Due to good hole-transport properties, the optionally substituted monocyclic, linked or fused aromatic hydrocarbon group with 6 to 30 carbon atoms or the optionally substituted monocyclic, linked or fused heteroaromatic group with 3 to 30 carbon atoms in Ar⁴ more preferably each independently denotes
(i′) a phenyl group, a biphenylyl group, a terphenylyl group, a naphthyl group, a fluorenyl group, a spirobifluorenyl group, a benzofluorenyl group, a phenanthryl group, a fluoranthenyl group, a triphenylenyl group, an anthryl group, a pyrenyl group, a dibenzofuranyl group or a dibenzothienyl group or
(ii′) the group represented by (i′) substituted with one or more groups selected from the group consisting of a methyl group, a phenyl group, a biphenylyl group, a naphthyl group, a phenanthryl group, a triphenylsilyl group, a carbazolyl group, a dibenzothienyl group and a dibenzofuranyl group.

Due to good hole-transport properties, Ar⁴ still more preferably each independently denotes
(iv) a phenyl group, a biphenylyl group, a terphenylyl group, a naphthyl group, a fluorenyl group, a spirobifluorenyl group, a benzofluorenyl group, a phenanthryl group, a fluoranthenyl group, a triphenylenyl group, an anthryl group, a pyrenyl group, a dibenzofuranyl group or a dibenzothienyl group,
(v) the group represented by (iv) substituted with one or more groups selected from the group consisting of a methyl group, an ethyl group, a methoxy group, an ethoxy group, a cyano group, a deuterium atom, a fluorine atom, a phenyl group, a biphenylyl group, a naphthyl group, a phenanthryl group, a triphenylsilyl group, a carbazolyl group, a dibenzothienyl group and a dibenzofuranyl group or
(vi) a group represented by any one of the formulae (24) to (35).

Ar⁴ particularly preferably each independently denotes a phenyl group, a methylphenyl group, a dimethylphenyl group, a biphenylyl group, a methylbiphenylyl group, a dimethylbiphenylyl group, a trimethylbiphenylyl group, a terphenylyl group, a methylterphenylyl group, a dimethylterphenylyl group, a naphthyl group, a 9,9-dimethylfluorenyl group, a 9,9-diphenylfluorenyl group, a spirobifluorenyl group, 11,11-dimethylbenzo[a]fluorene, 11,11-dimethylbenzo[b]fluorene, 7,7-dimethylbenzo[c]fluorene, a phenanthryl group, a fluoranthenyl group, a triphenylenyl group, a naphthyl phenyl group, a phenanthryl phenyl group, a triphenylsilyl phenyl group, a carbazolyl phenyl group, a dibenzofuranyl group, a dibenzothienyl group, a dibenzofuranyl phenyl group or a dibenzothienyl phenyl group.

In the formulae (24) to (40), R⁴ preferably denotes a biphenylyl group, a methylbiphenylyl group, a dimethylbiphenylyl group, a naphthyl group, a phenanthryl group, a dibenzofuranyl group or a dibenzothienyl group.

In the formulae (24) to (40), R⁶ preferably denotes a phenyl group, a methylphenyl group, a dimethylphenyl group, a trimethylphenyl group, a biphenylyl group, a methylbiphenylyl group, a dimethylbiphenylyl group, a naphthyl group, a phenanthryl group, a dibenzofuranyl group or a dibenzothienyl group.

In the formulae (24) to (40), preferably, R⁷ and R⁸ each independently denote a phenyl group, a methylphenyl group, a dimethylphenyl group, a trimethylphenyl group, a biphenylyl group, a methylbiphenylyl group, a dimethylbiphenylyl group, a naphthyl group, a phenanthryl group, a dibenzofuranyl group or a dibenzothienyl group, and at least one of R⁷ and R⁸ denotes a biphenylyl group, a methylbiphenylyl group, a dimethylbiphenylyl group, a naphthyl group, a phenanthryl group, a dibenzofuranyl group or a dibenzothienyl group.

Due to the effect of suppressing a transverse current, the formulae (24) to (40) are preferably one selected from the groups represented by the formulae (Z1) to (Z209).

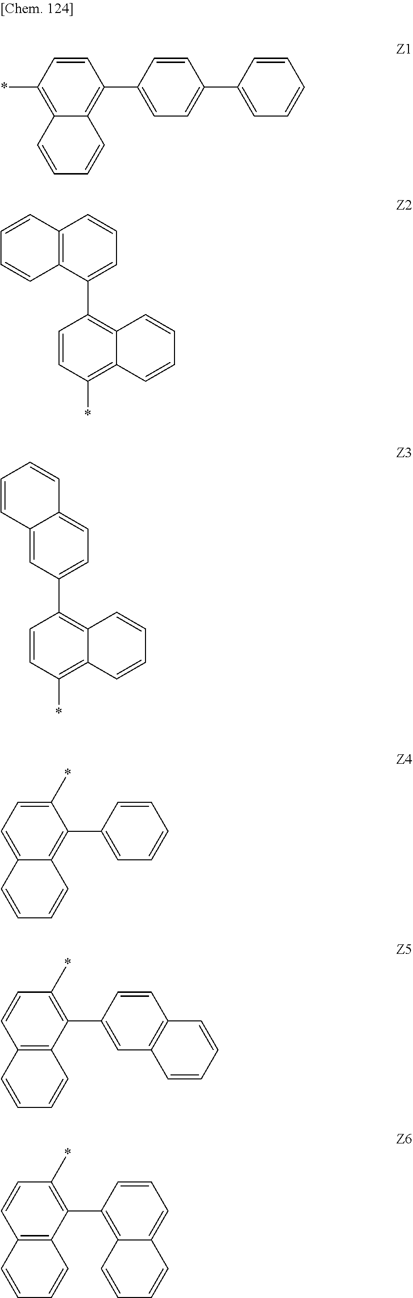

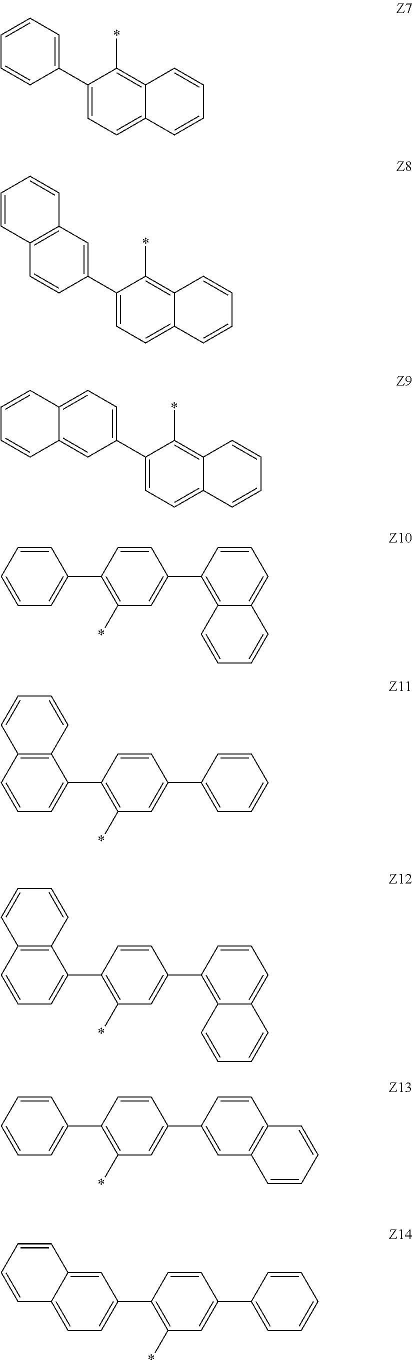

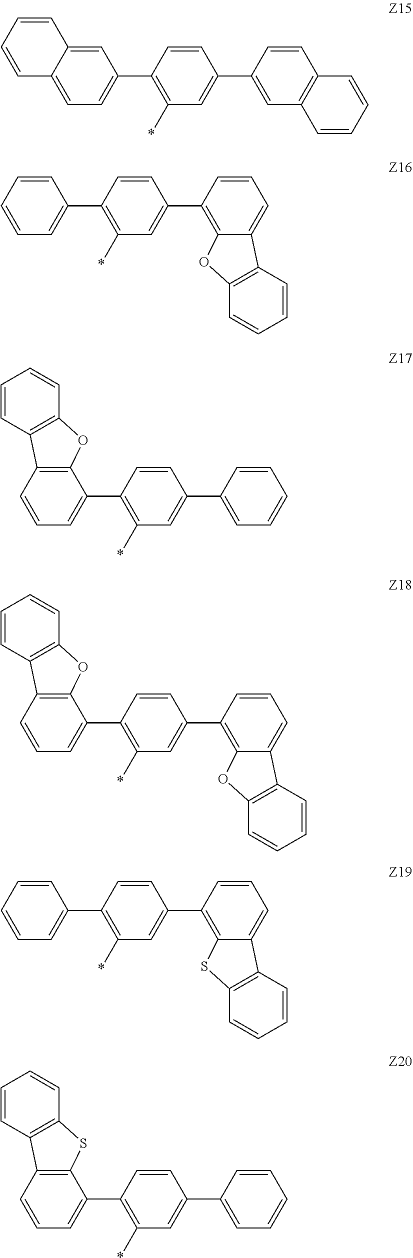

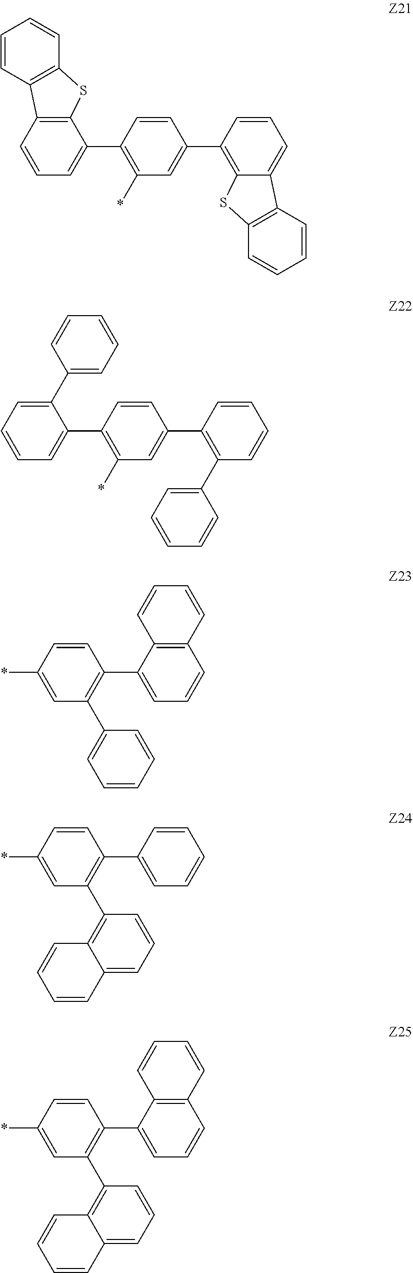

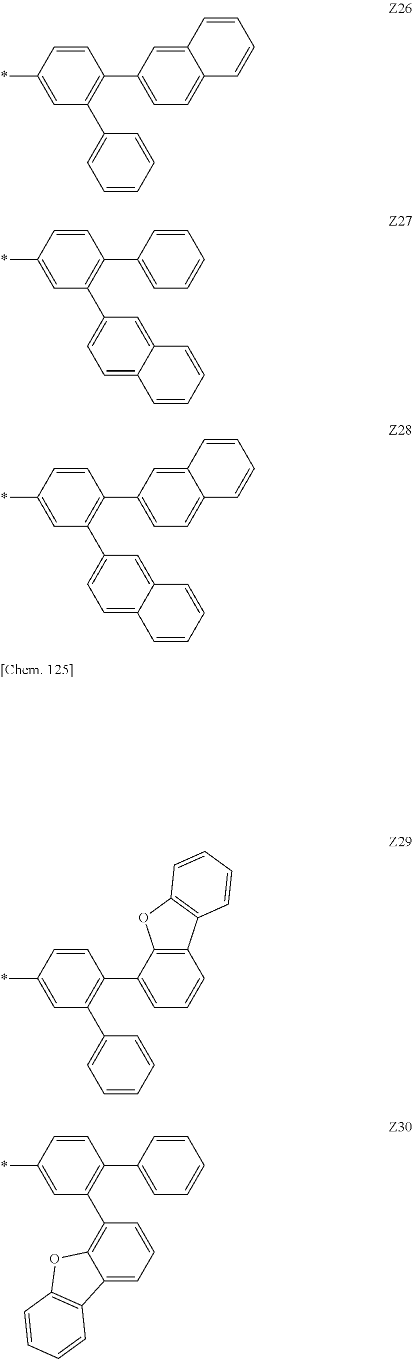

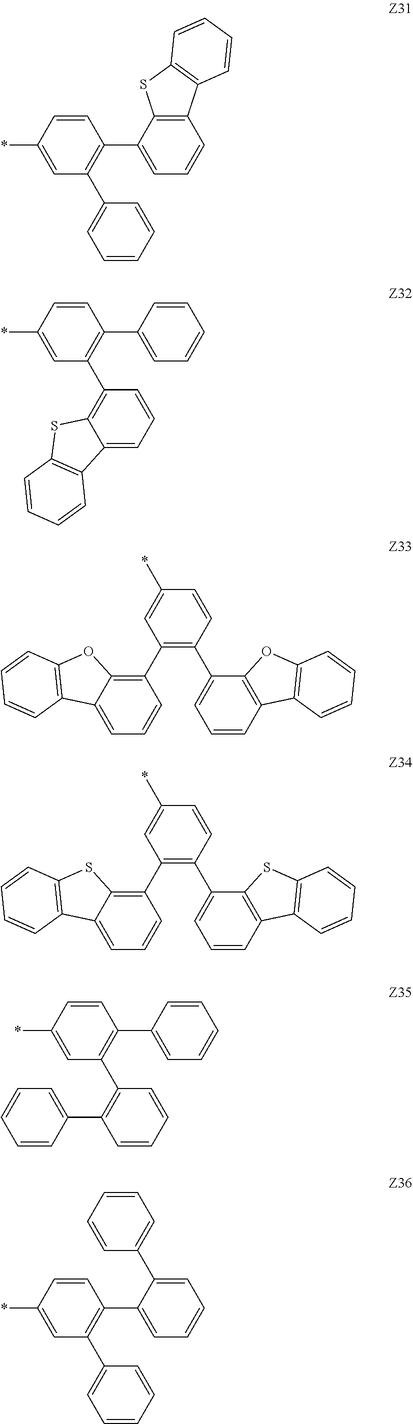

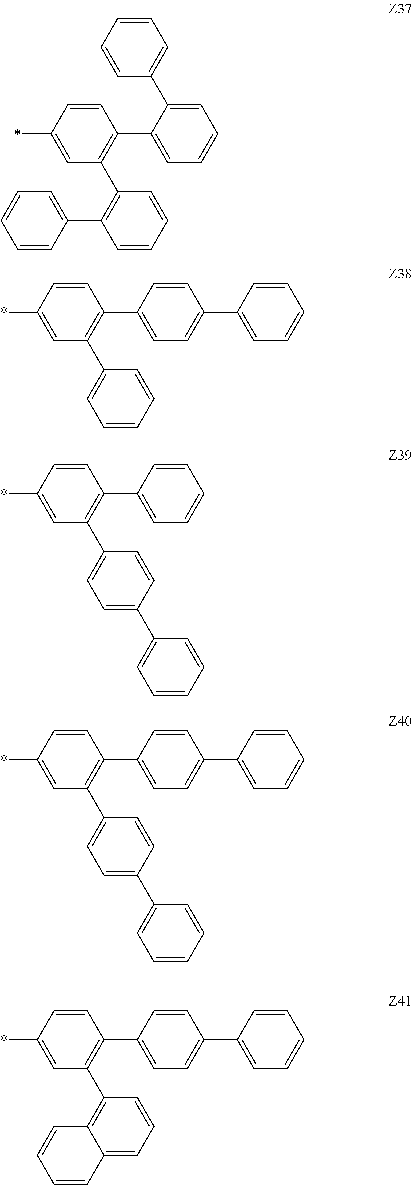

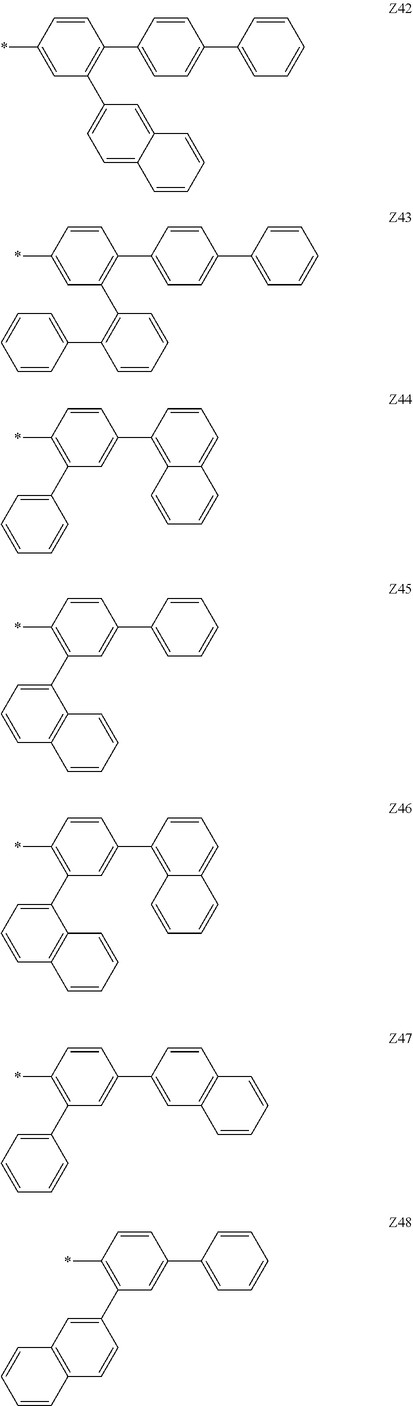

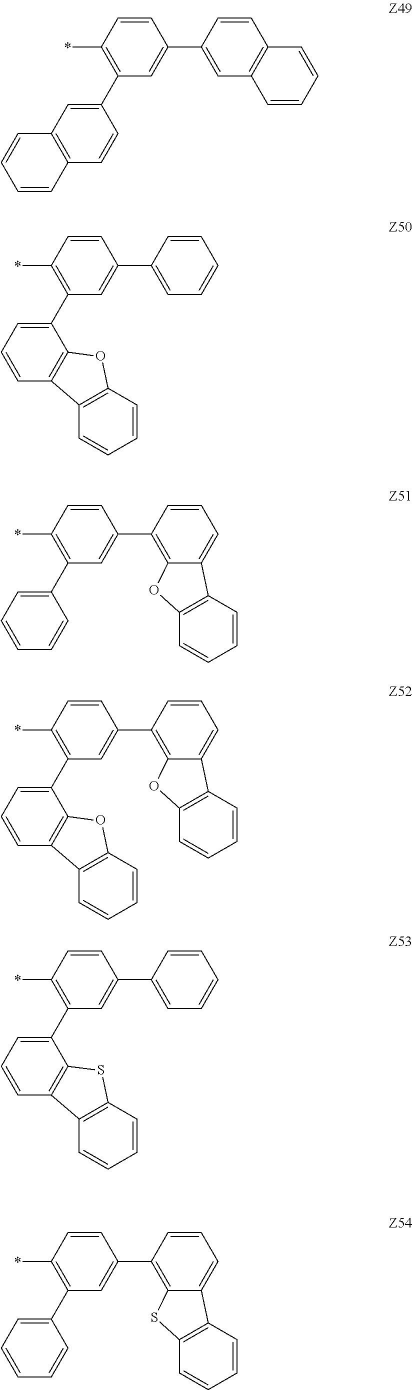

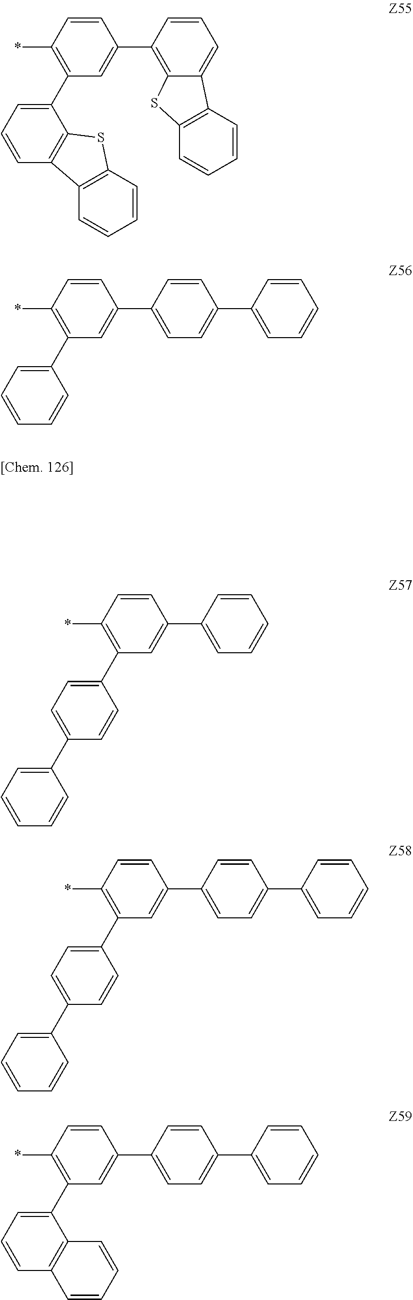

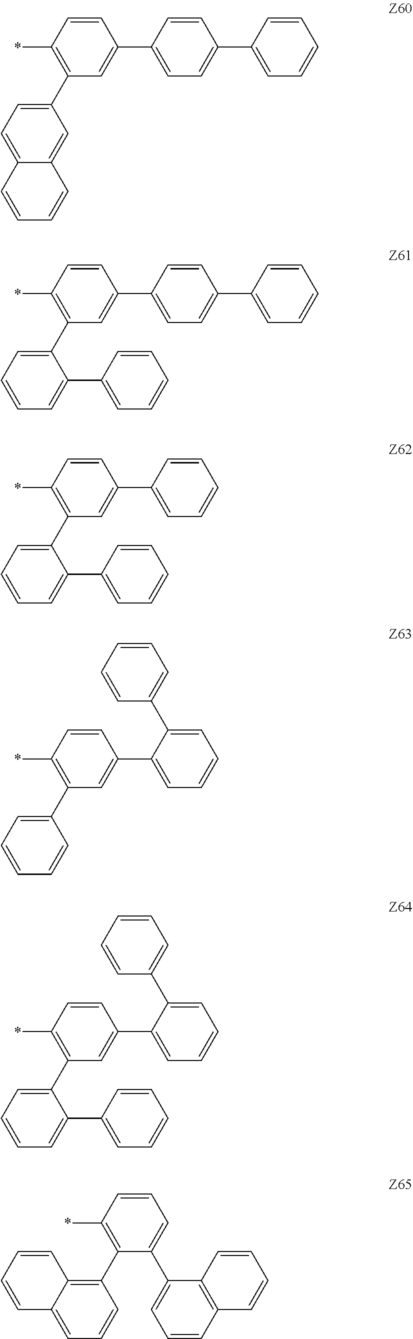

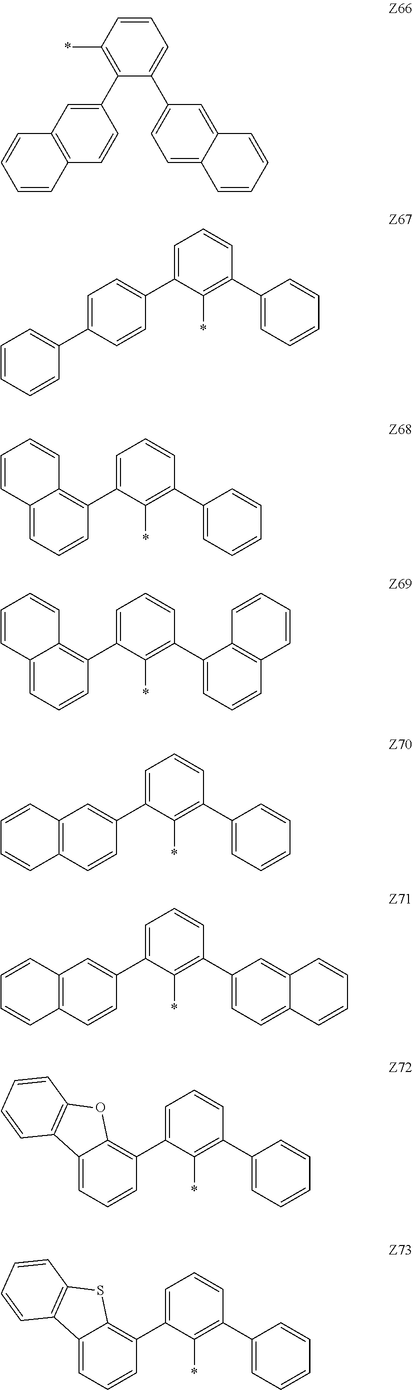

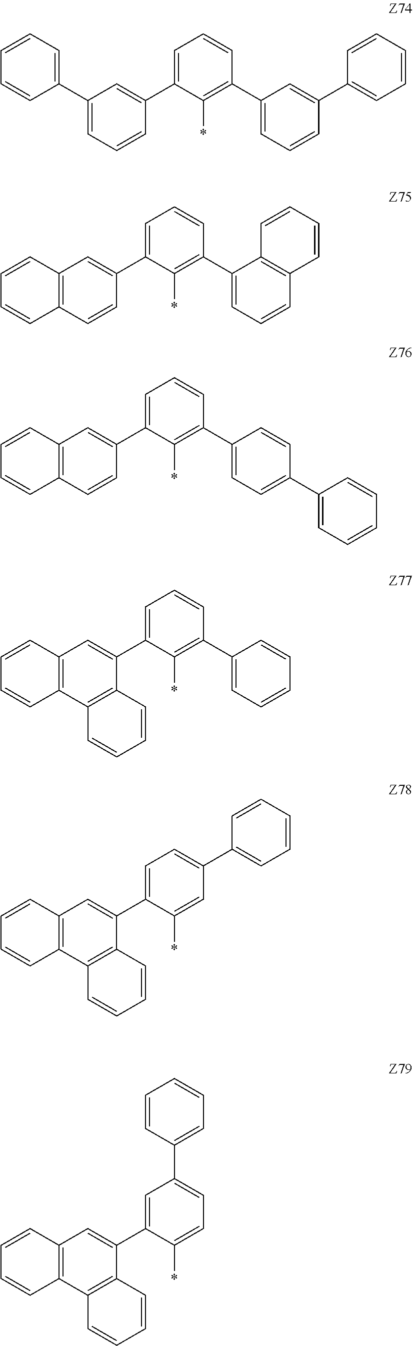 [SMILES: *c1c(-c2cccc(-c3ccccc3)c2)cccc1-c1cccc(-c2ccccc2)c1 ; *c1c(-c2ccc3ccccc3c2)cccc1-c1cccc2ccccc12 ; *c1c(-c2ccc(-c3ccccc3)cc2)cccc1-c1ccc2ccccc2c1 ; *c1c(-c2ccccc2)cccc1-c1cc2ccccc2c2ccccc12 ; *c1cc(-c2ccccc2)ccc1-c1cc2ccccc2c2ccccc12 ; *c1ccc(-c2ccccc2)cc1-c1cc2ccccc2c2ccccc12]

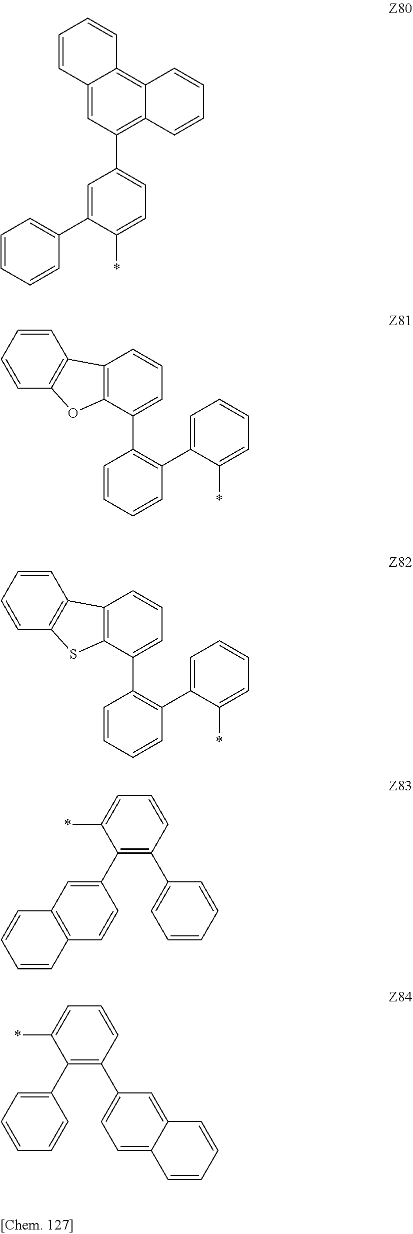

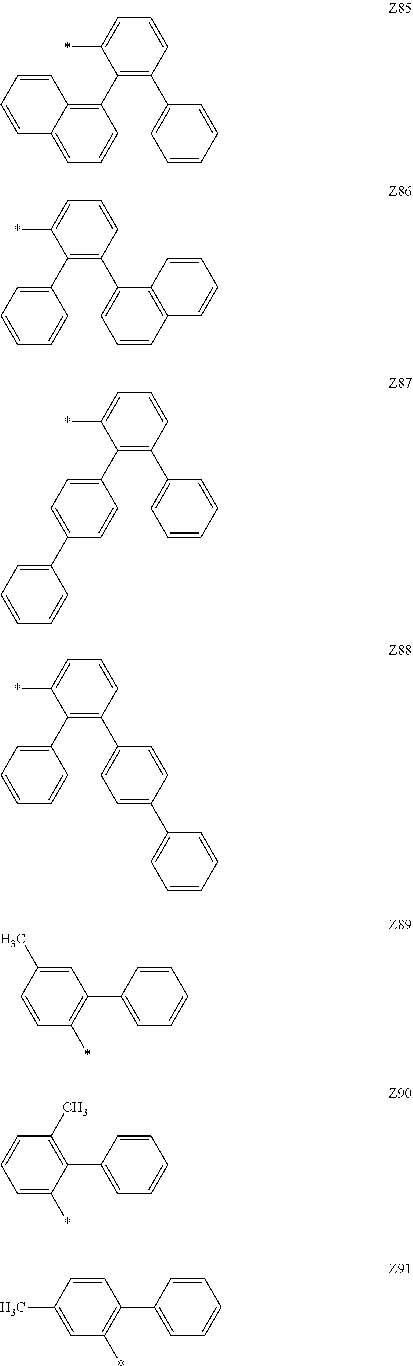

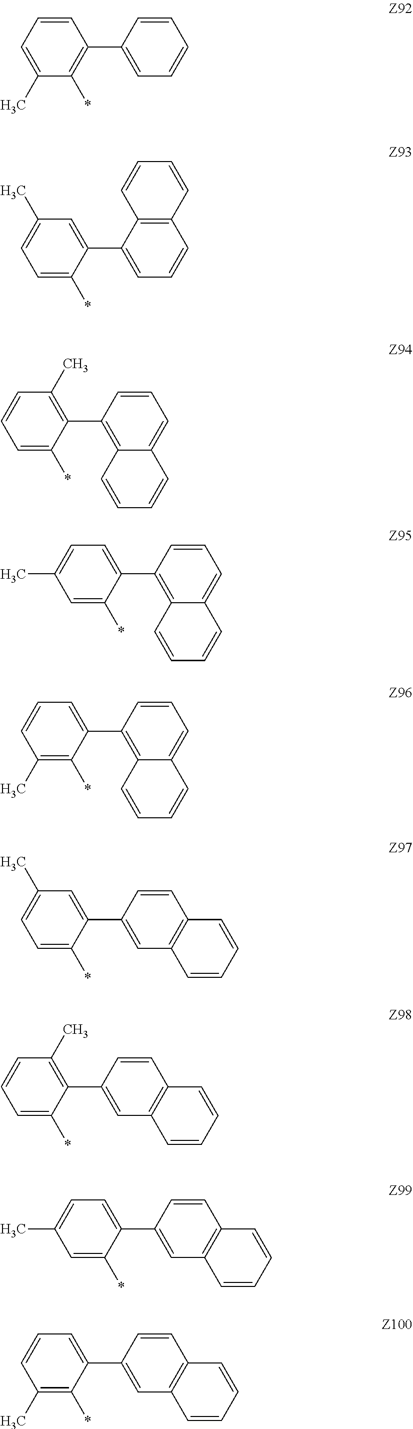

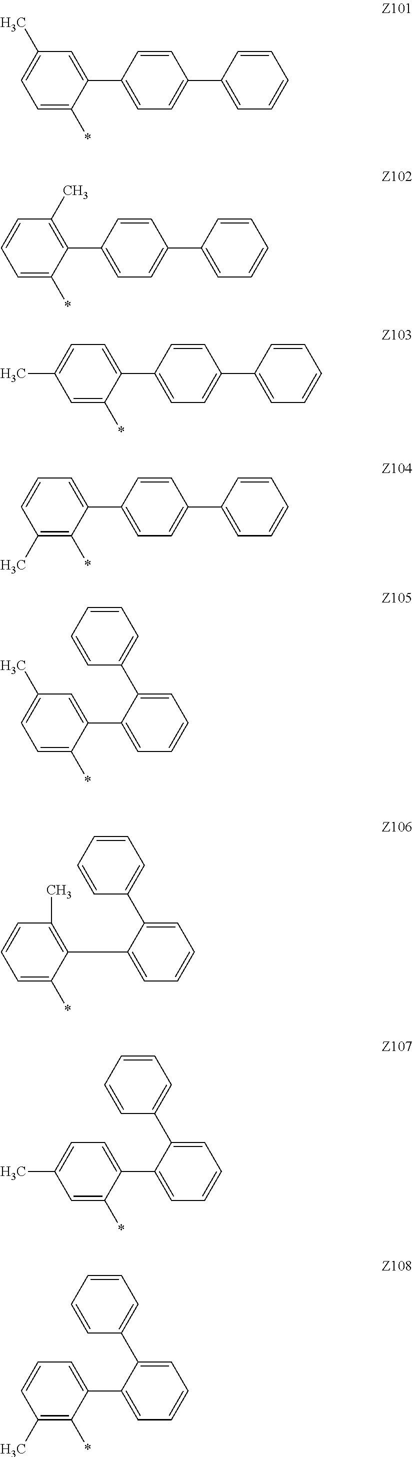

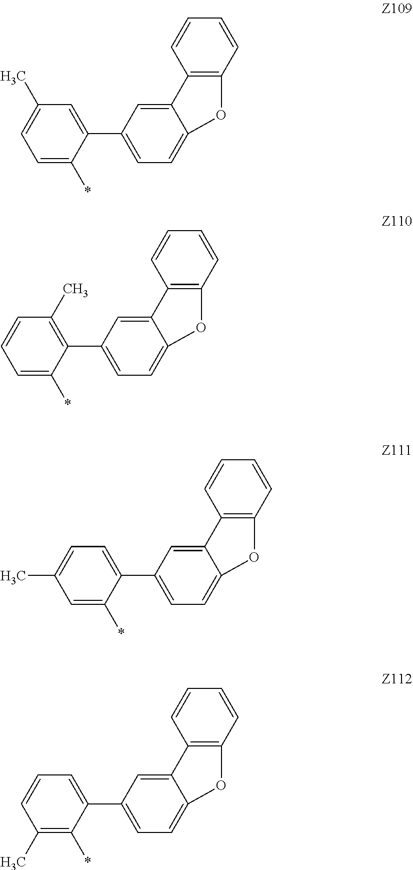 [SMILES: *c1ccc(C)cc1-c1ccc2oc3ccccc3c2c1 ; *c1cccc(C)c1-c1ccc2oc3ccccc3c2c1 ; *c1cc(C)ccc1-c1ccc2oc3ccccc3c2c1 ; *c1c(C)cccc1-c1ccc2oc3ccccc3c2c1]

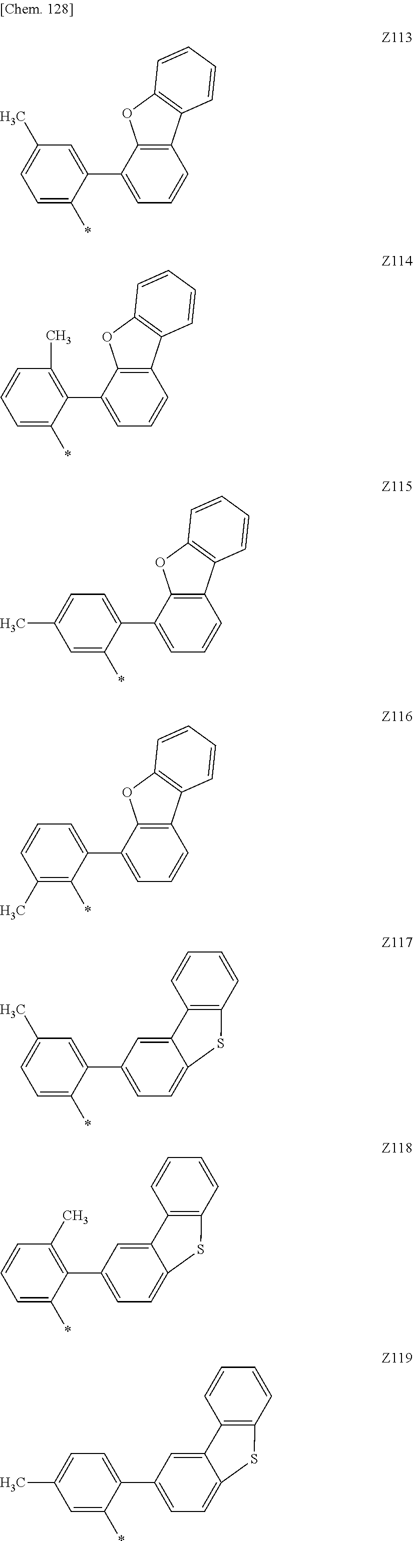

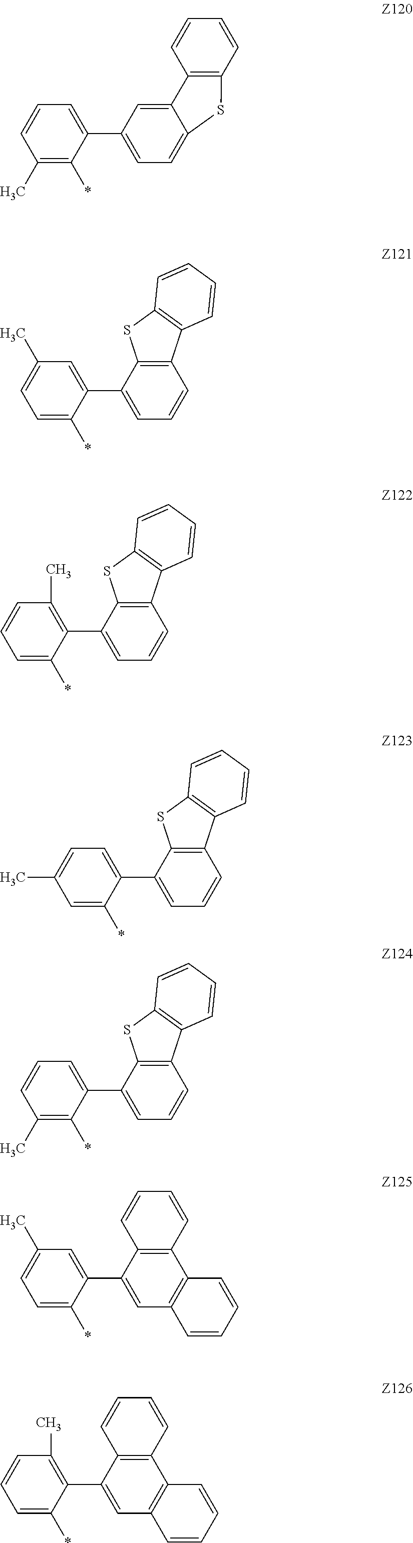

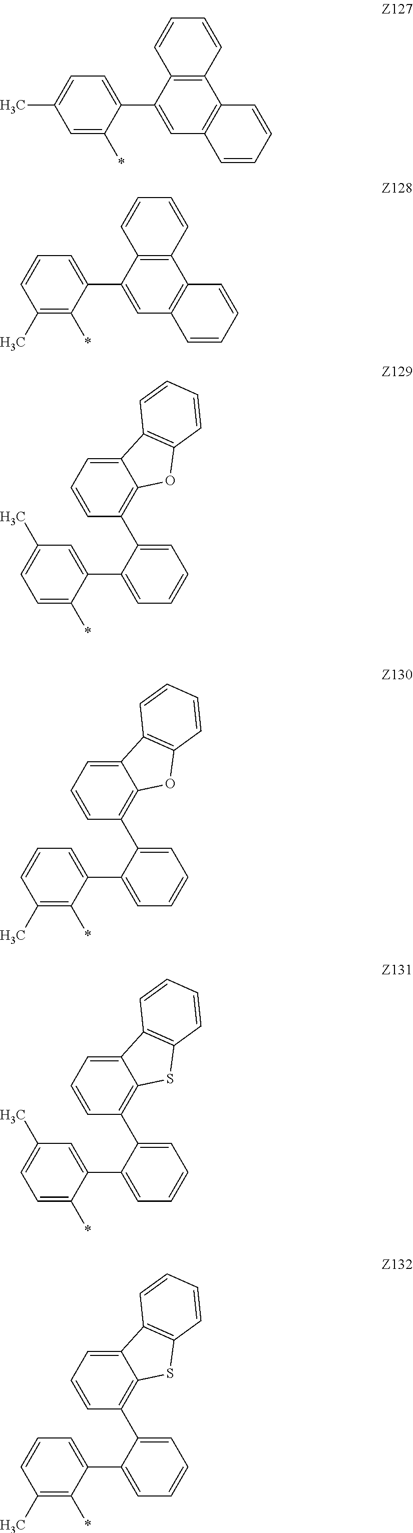

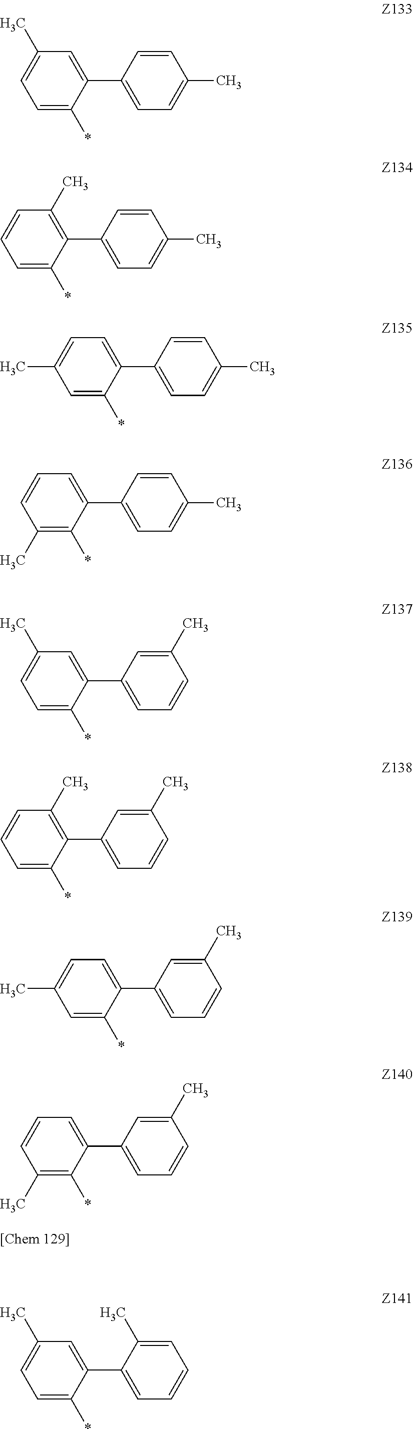

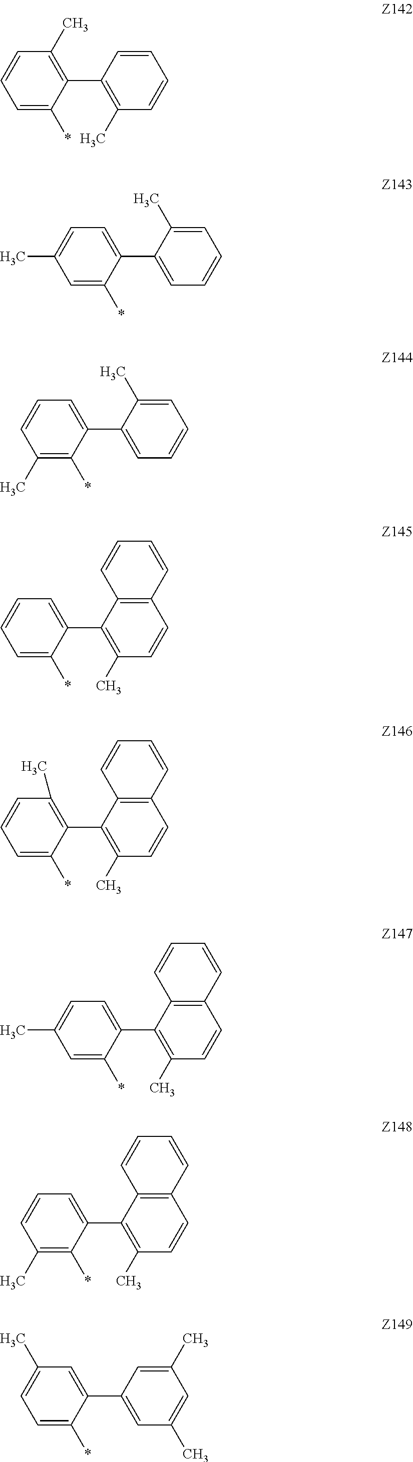

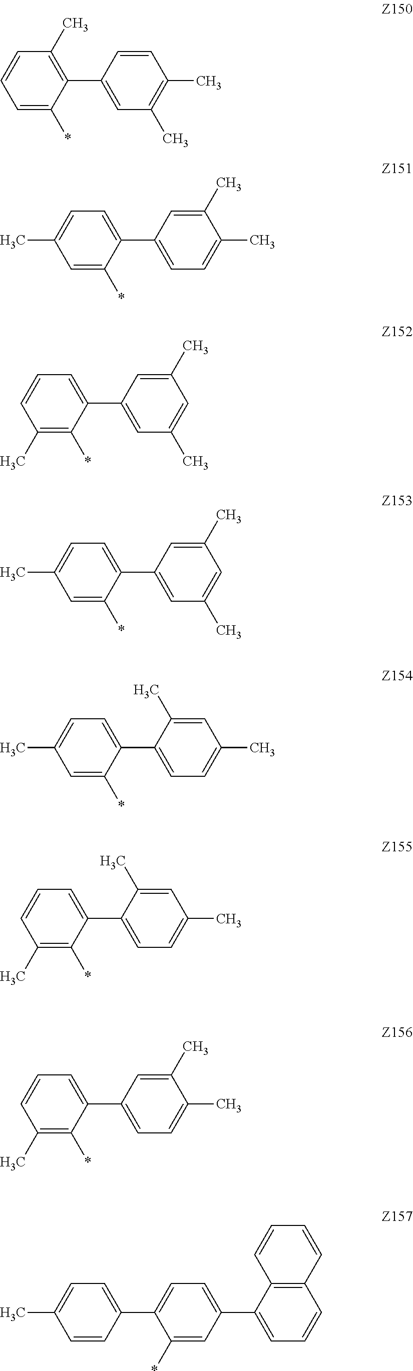

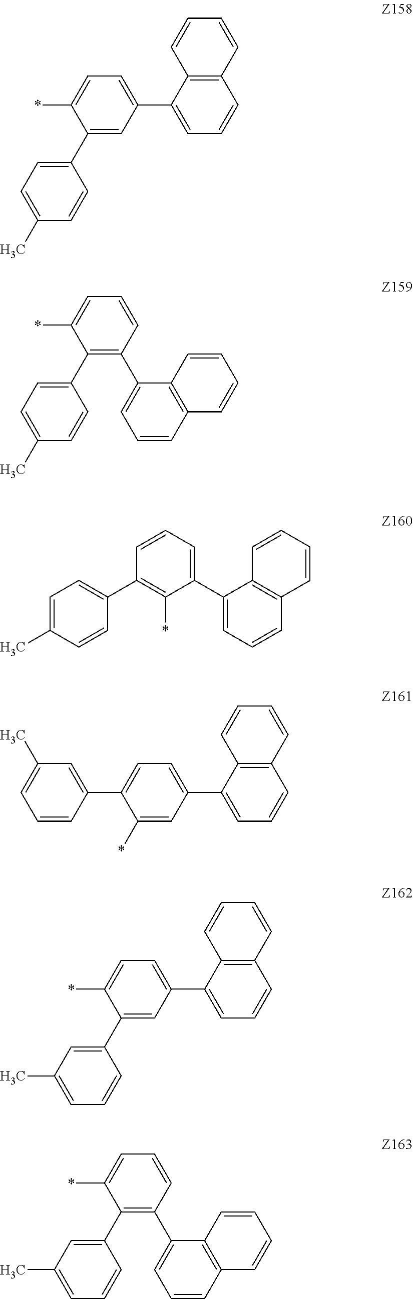 [SMILES: *c1ccc(-c2cccc3ccccc23)cc1-c1ccc(C)cc1 ; *c1cccc(-c2cccc3ccccc23)c1-c1ccc(C)cc1 ; *c1c(-c2ccc(C)cc2)cccc1-c1cccc2ccccc12 ; *c1cc(-c2cccc3ccccc23)ccc1-c1cccc(C)c1 ; *c1ccc(-c2cccc3ccccc23)cc1-c1cccc(C)c1 ; *c1cccc(-c2cccc3ccccc23)c1-c1cccc(C)c1]

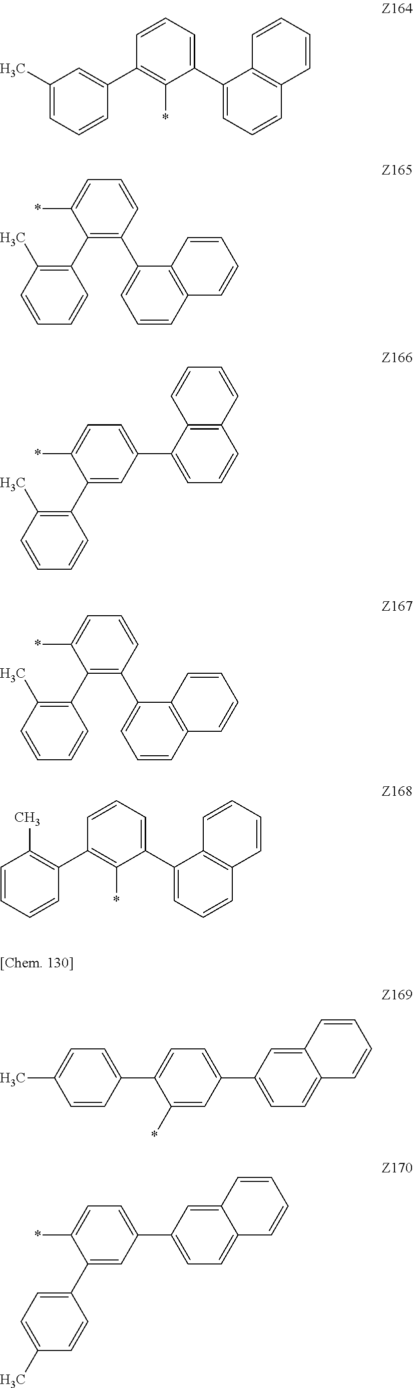

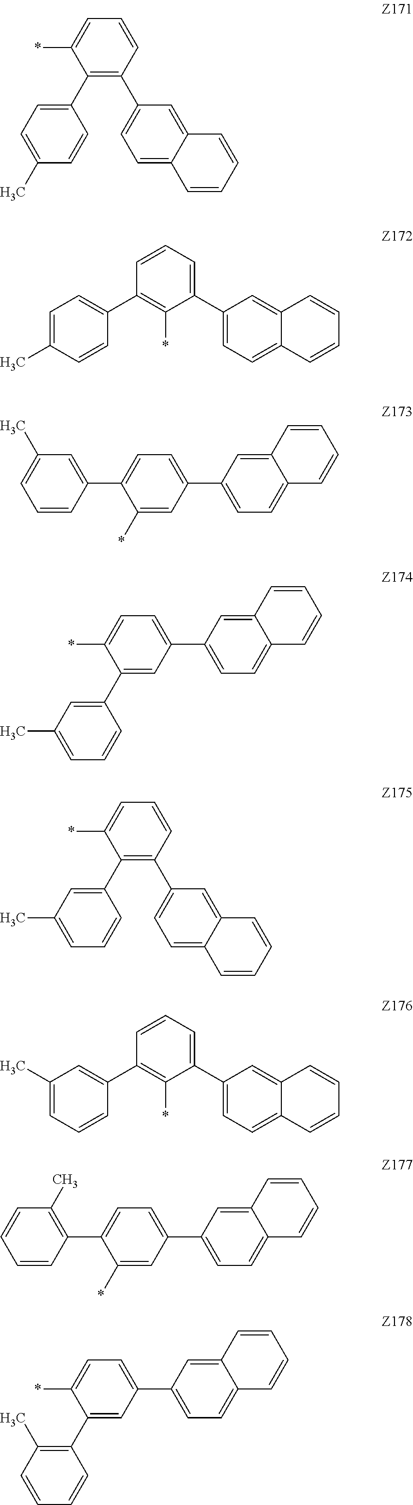

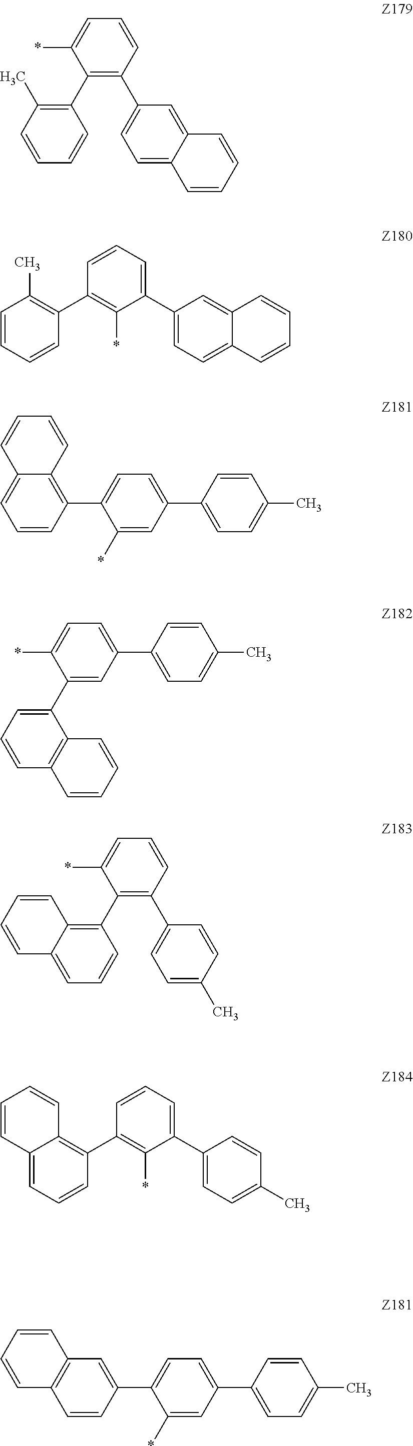

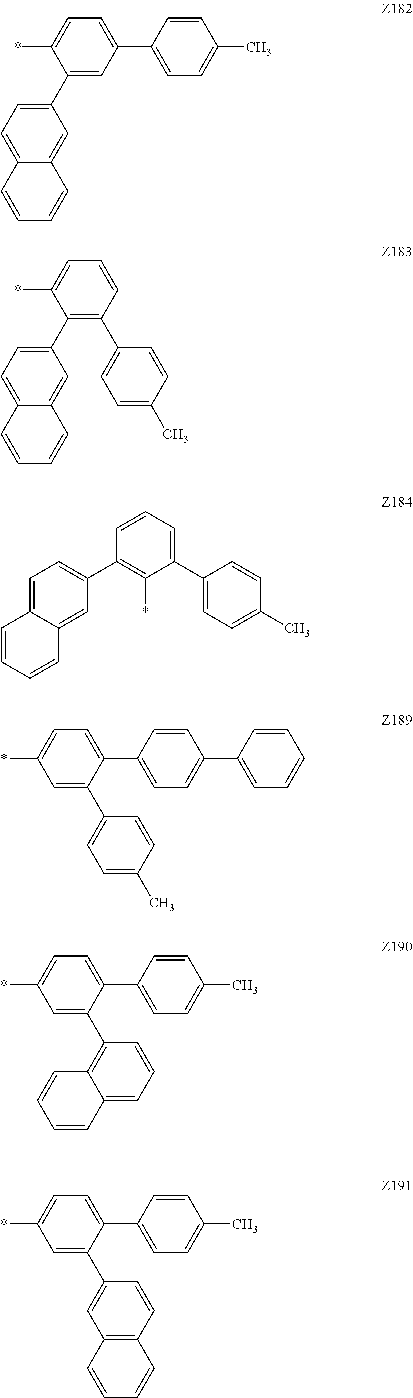

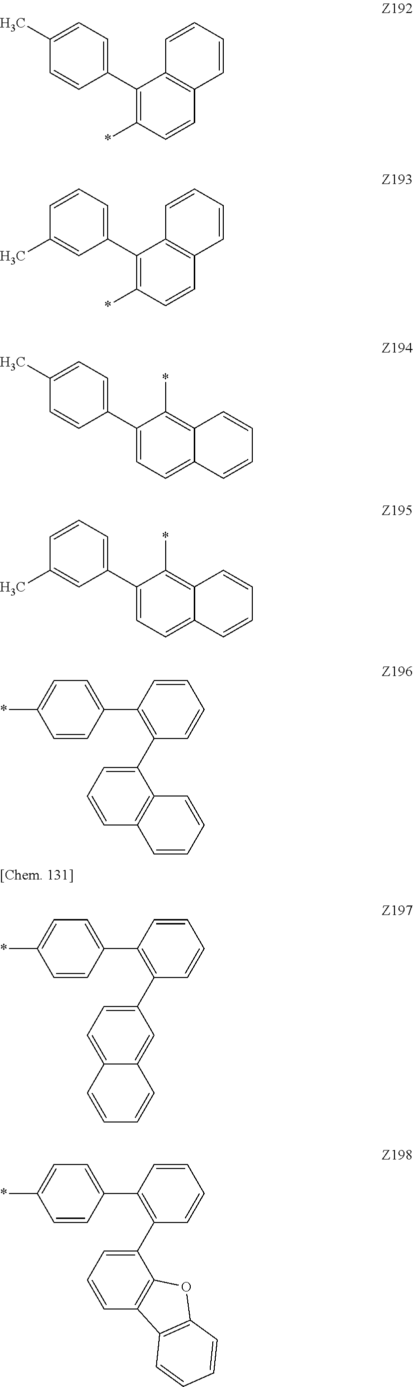

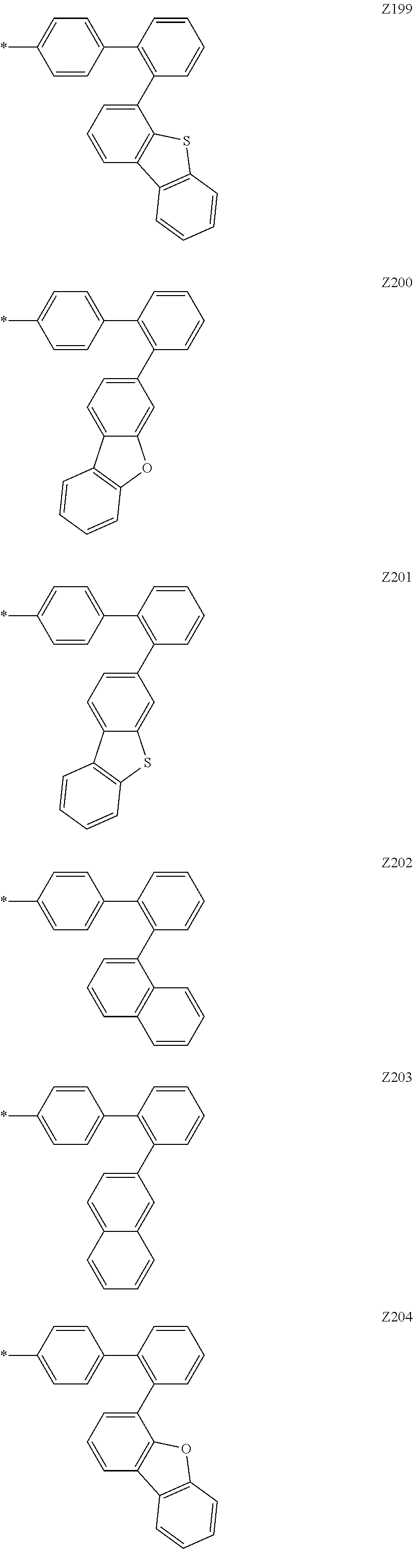

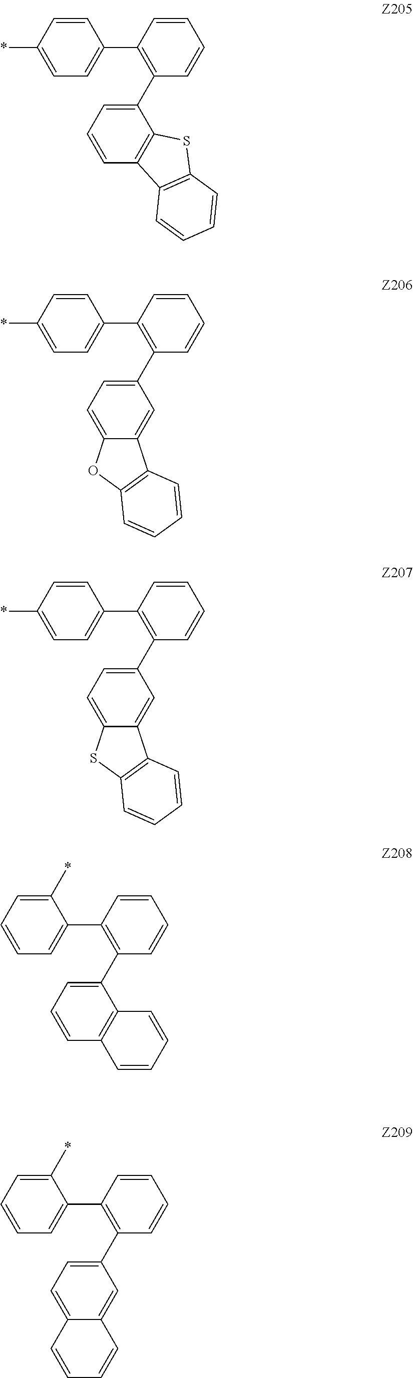

### [Specific Examples of Transverse Current Suppressing Material and Carbazole Compound]

For a transverse current suppressing material according to an embodiment of the present disclosure and a carbazole compound according to an embodiment of the present disclosure, although the compounds represented by the formulae (D1) to (D859), (E1) to (E772), (F1) to (F924) and (G1) to (G718) are exemplified below, the present disclosure is not limited to these compounds.

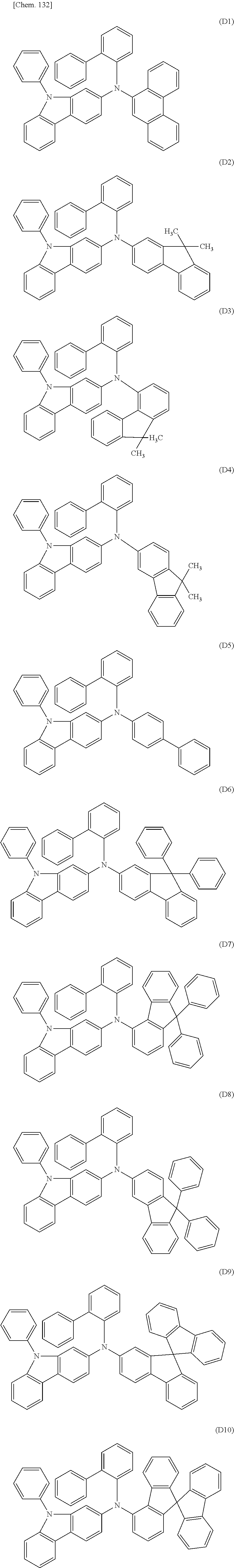

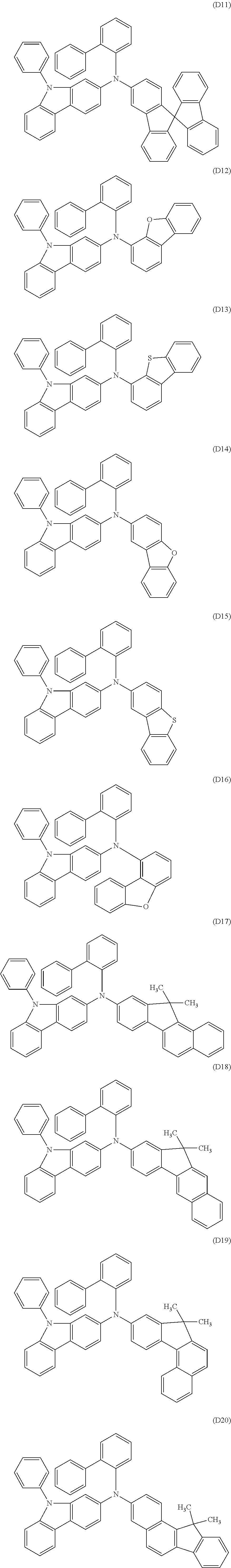

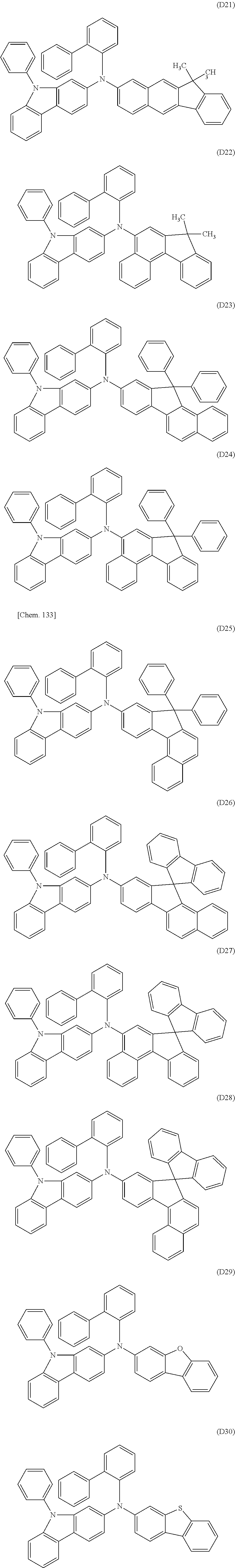

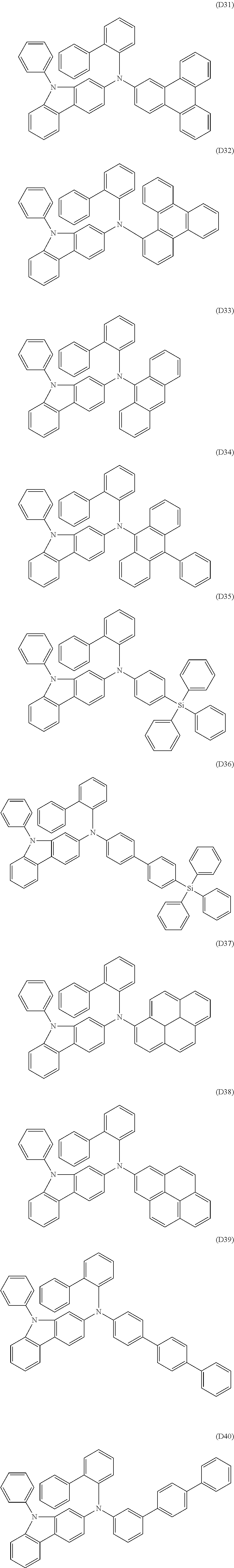

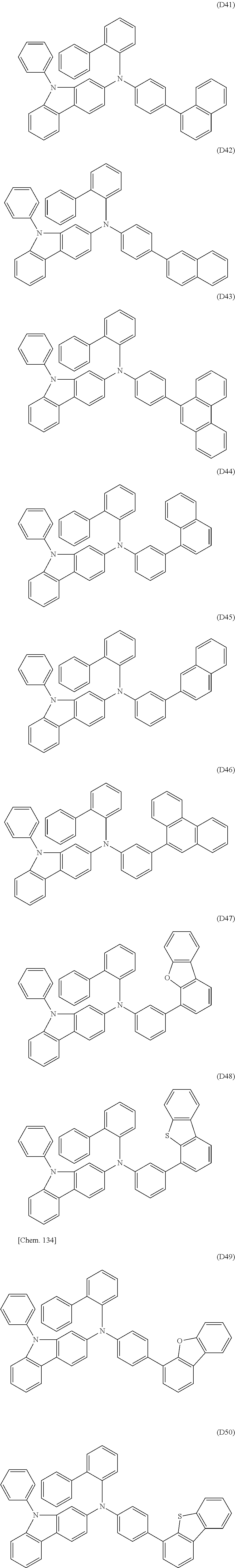

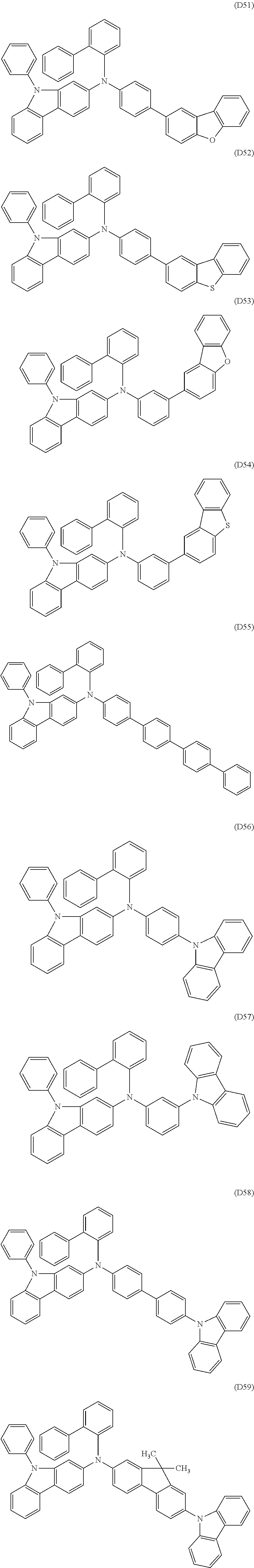

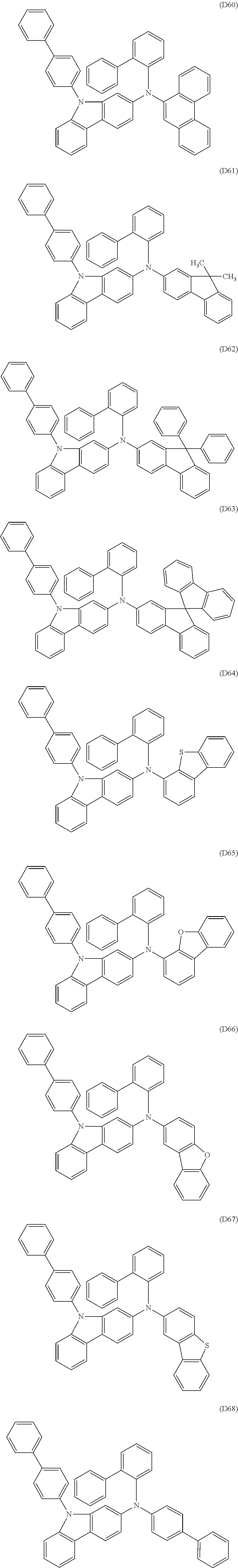

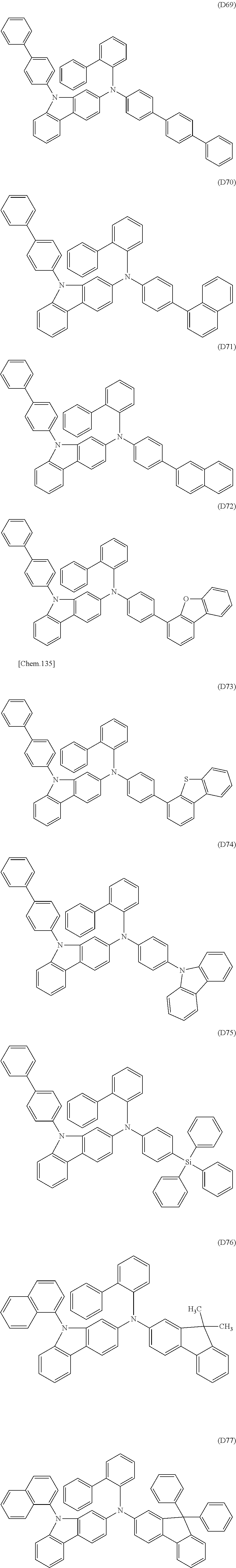

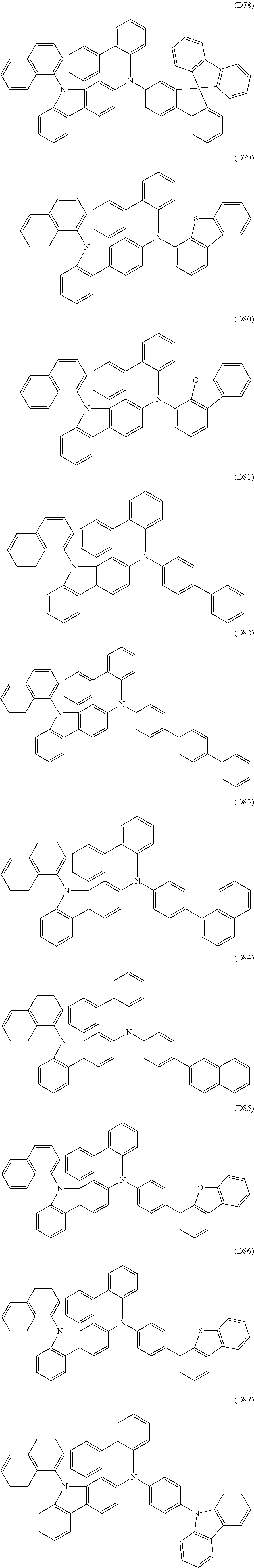

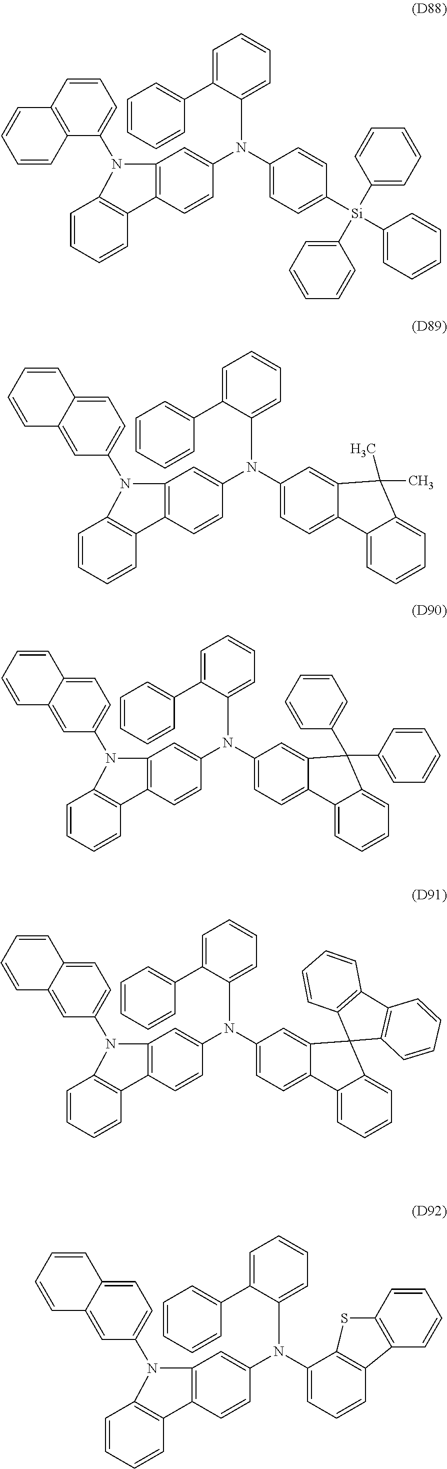 [SMILES: c1ccc(-c2ccccc2N(c2ccc([Si](c3ccccc3)(c3ccccc3)c3ccccc3)cc2)c2ccc3c4ccccc4n(-c4cccc5ccccc45)c3c2)cc1 ; CC1(C)c2ccccc2-c2ccc(N(c3ccc4c5ccccc5n(-c5ccc6ccccc6c5)c4c3)c3ccccc3-c3ccccc3)cc21 ; c1ccc(-c2ccccc2N(c2ccc3c(c2)C(c2ccccc2)(c2ccccc2)c2ccccc2-3)c2ccc3c4ccccc4n(-c4ccc5ccccc5c4)c3c2)cc1 ; c1ccc(-c2ccccc2N(c2ccc3c(c2)C2(c4ccccc4-c4ccccc42)c2ccccc2-3)c2ccc3c4ccccc4n(-c4ccc5ccccc5c4)c3c2)cc1 ; c1ccc(-c2ccccc2N(c2ccc3c4ccccc4n(-c4ccc5ccccc5c4)c3c2)c2cccc3c2sc2ccccc23)cc1]

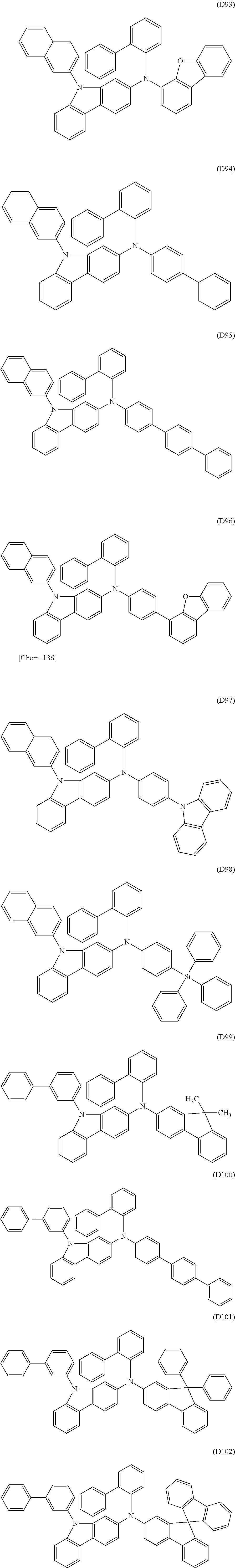

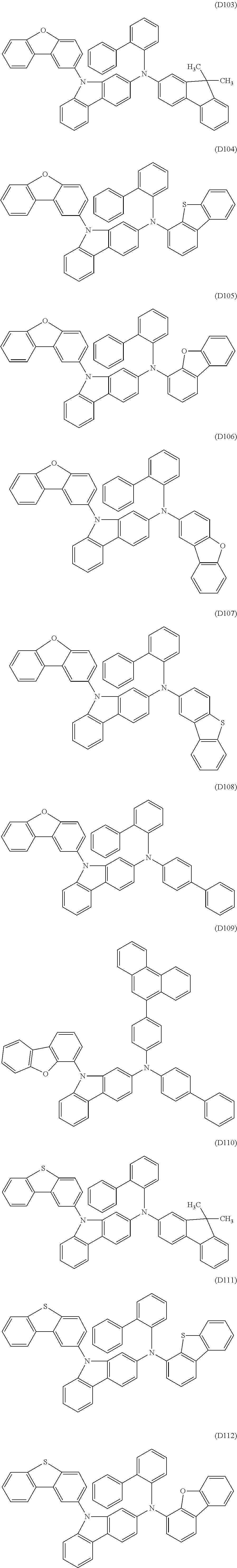

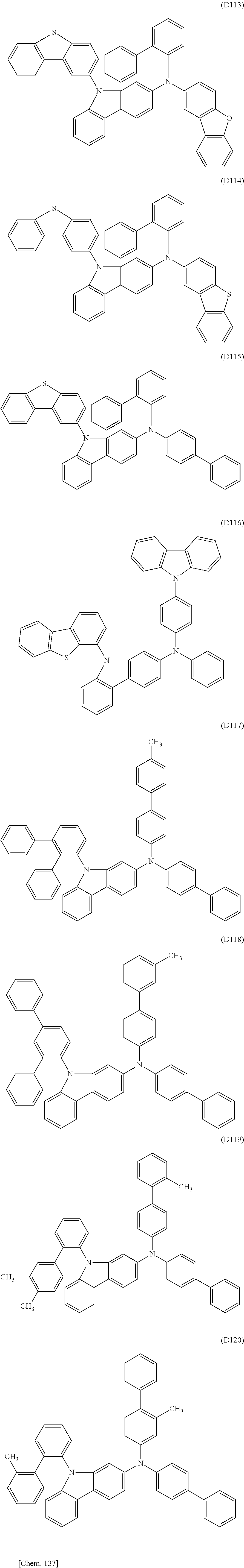

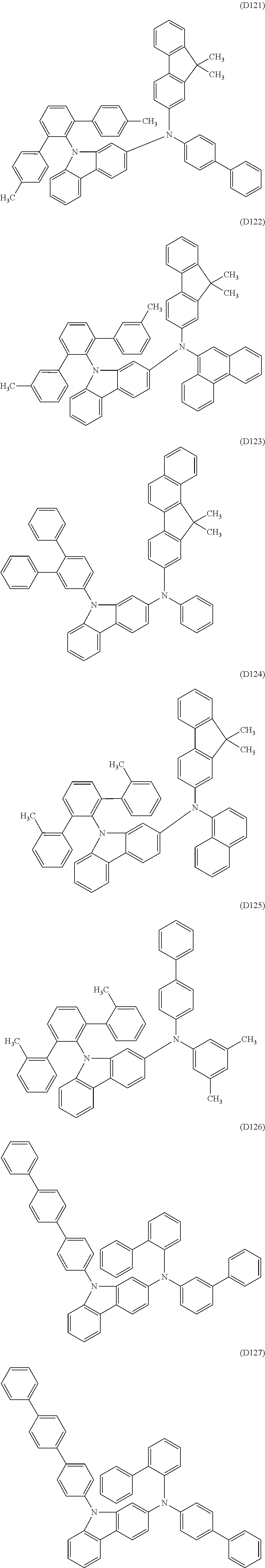

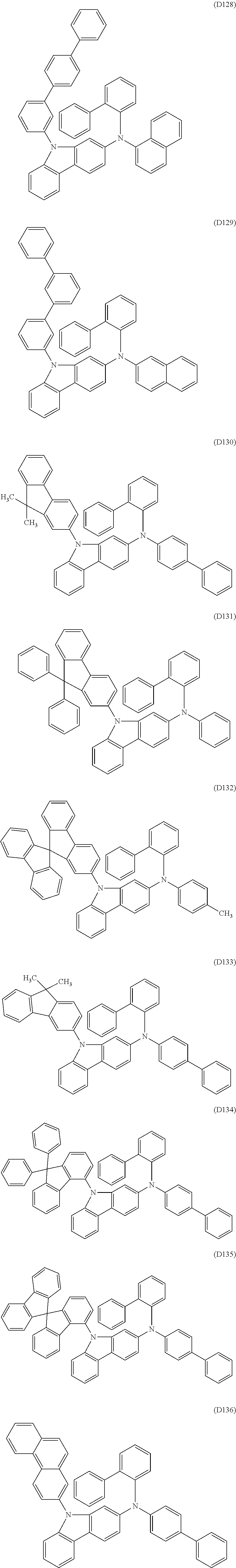

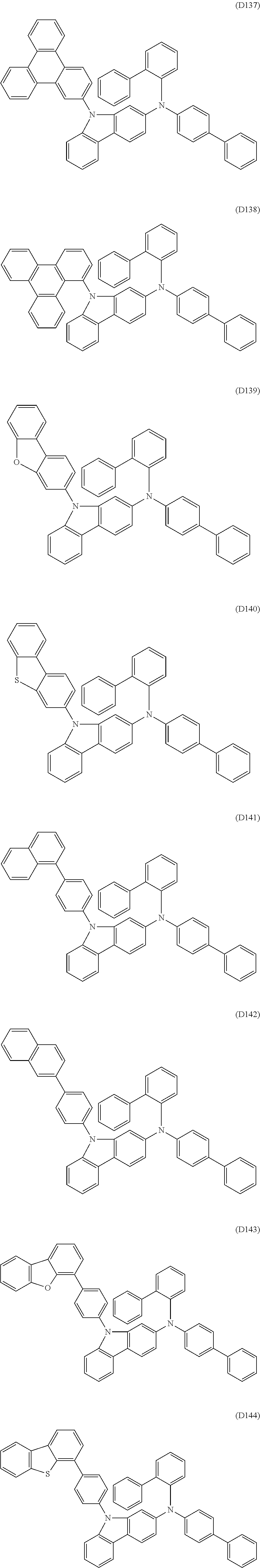

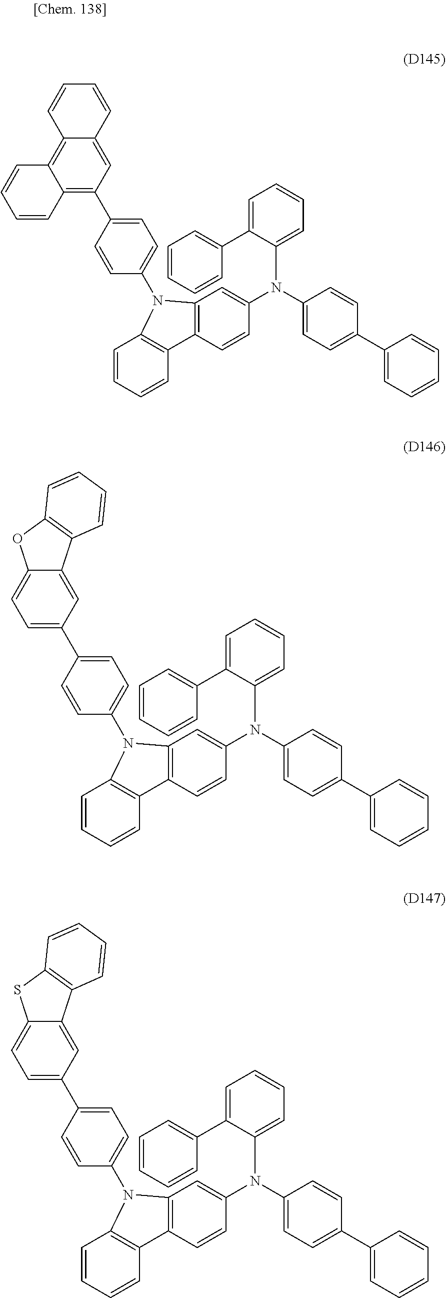 [SMILES: c1ccc(-c2ccc(N(c3ccc4c5ccccc5n(-c5ccc(-c6cc7ccccc7c7ccccc67)cc5)c4c3)c3ccccc3-c3ccccc3)cc2)cc1 ; c1ccc(-c2ccc(N(c3ccc4c5ccccc5n(-c5ccc(-c6ccc7oc8ccccc8c7c6)cc5)c4c3)c3ccccc3-c3ccccc3)cc2)cc1 ; c1ccc(-c2ccc(N(c3ccc4c5ccccc5n(-c5ccc(-c6ccc7sc8ccccc8c7c6)cc5)c4c3)c3ccccc3-c3ccccc3)cc2)cc1]

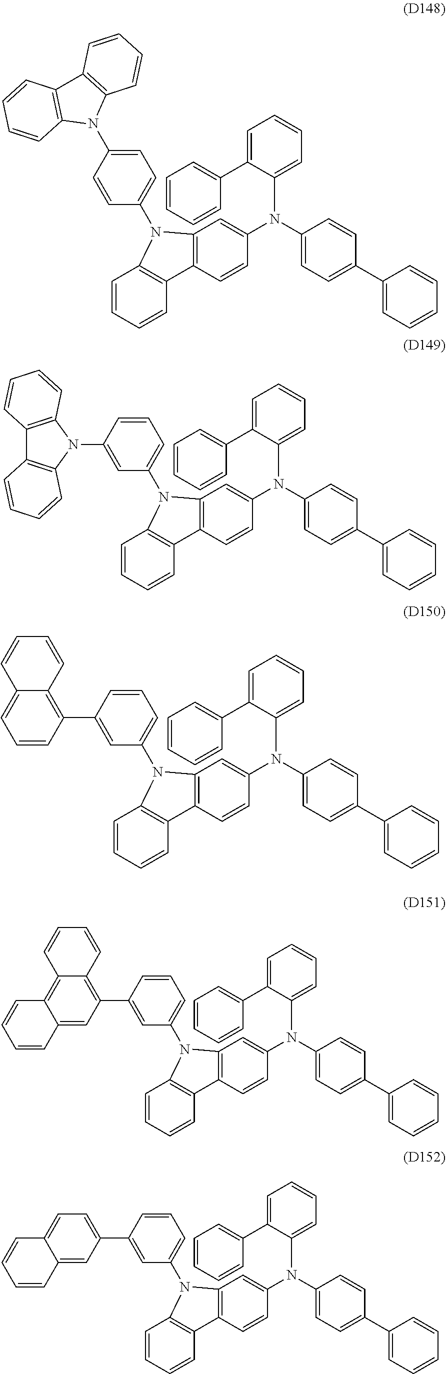 [SMILES: c1ccc(-c2ccc(N(c3ccc4c5ccccc5n(-c5ccc(-n6c7ccccc7c7ccccc76)cc5)c4c3)c3ccccc3-c3ccccc3)cc2)cc1 ; c1ccc(-c2ccc(N(c3ccc4c5ccccc5n(-c5cccc(-n6c7ccccc7c7ccccc76)c5)c4c3)c3ccccc3-c3ccccc3)cc2)cc1 ; c1ccc(-c2ccc(N(c3ccc4c5ccccc5n(-c5cccc(-c6cccc7ccccc67)c5)c4c3)c3ccccc3-c3ccccc3)cc2)cc1 ; c1ccc(-c2ccc(N(c3ccc4c5ccccc5n(-c5cccc(-c6cc7ccccc7c7ccccc67)c5)c4c3)c3ccccc3-c3ccccc3)cc2)cc1 ; c1ccc(-c2ccc(N(c3ccc4c5ccccc5n(-c5cccc(-c6ccc7ccccc7c6)c5)c4c3)c3ccccc3-c3ccccc3)cc2)cc1]

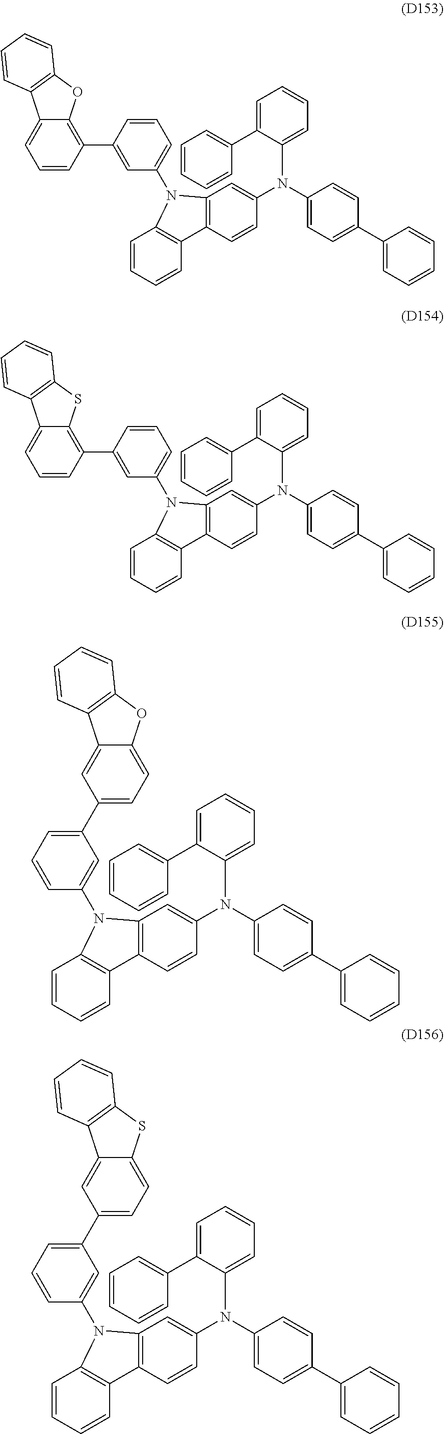

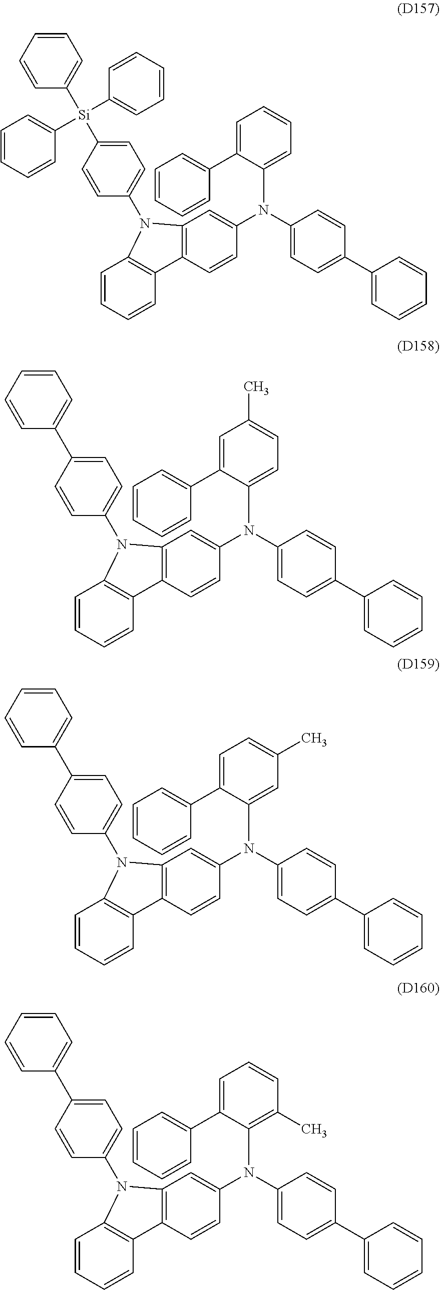 [SMILES: c1ccc(-c2ccc(N(c3ccc4c5ccccc5n(-c5ccc([Si](c6ccccc6)(c6ccccc6)c6ccccc6)cc5)c4c3)c3ccccc3-c3ccccc3)cc2)cc1 ; Cc1ccc(N(c2ccc(-c3ccccc3)cc2)c2ccc3c4ccccc4n(-c4ccc(-c5ccccc5)cc4)c3c2)c(-c2ccccc2)c1 ; Cc1ccc(-c2ccccc2)c(N(c2ccc(-c3ccccc3)cc2)c2ccc3c4ccccc4n(-c4ccc(-c5ccccc5)cc4)c3c2)c1 ; Cc1cccc(-c2ccccc2)c1N(c1ccc(-c2ccccc2)cc1)c1ccc2c3ccccc3n(-c3ccc(-c4ccccc4)cc3)c2c1]

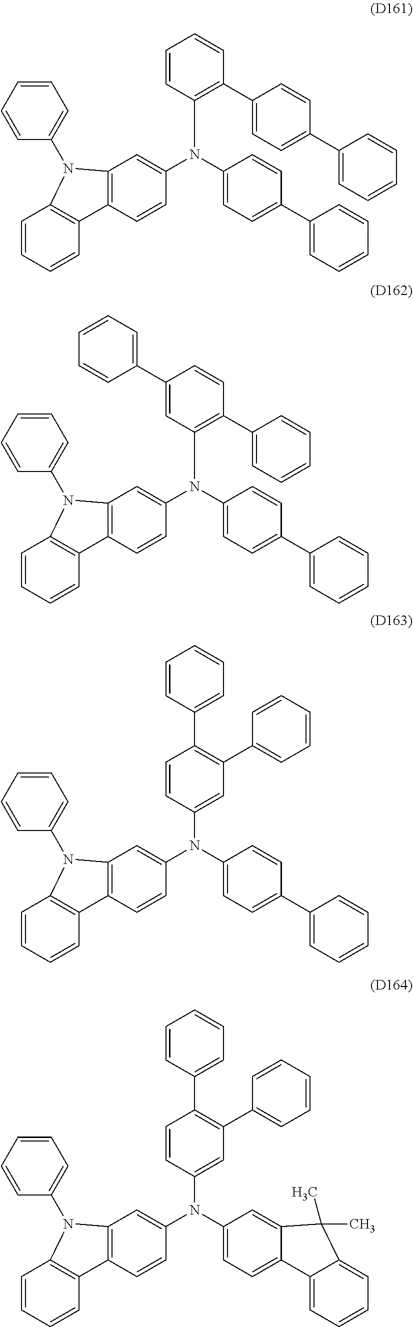 [SMILES: c1ccc(-c2ccc(-c3ccccc3N(c3ccc(-c4ccccc4)cc3)c3ccc4c5ccccc5n(-c5ccccc5)c4c3)cc2)cc1 ; c1ccc(-c2ccc(N(c3ccc4c5ccccc5n(-c5ccccc5)c4c3)c3cc(-c4ccccc4)ccc3-c3ccccc3)cc2)cc1 ; c1ccc(-c2ccc(N(c3ccc(-c4ccccc4)c(-c4ccccc4)c3)c3ccc4c5ccccc5n(-c5ccccc5)c4c3)cc2)cc1 ; CC1(C)c2ccccc2-c2ccc(N(c3ccc(-c4ccccc4)c(-c4ccccc4)c3)c3ccc4c5ccccc5n(-c5ccccc5)c4c3)cc21]

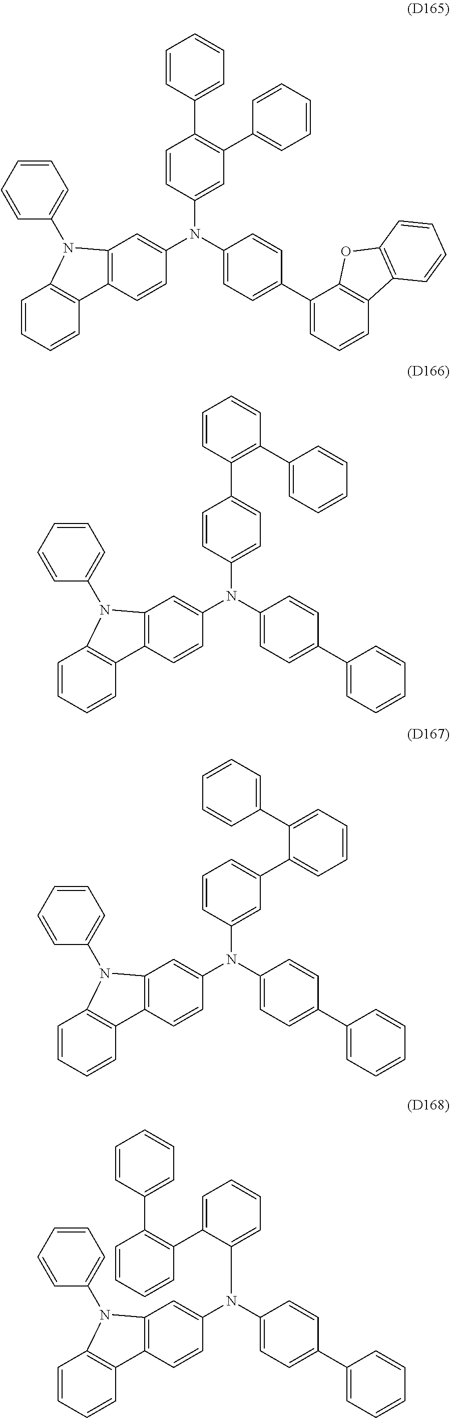 [SMILES: c1ccc(-c2ccc(N(c3ccc(-c4cccc5c4oc4ccccc45)cc3)c3ccc4c5ccccc5n(-c5ccccc5)c4c3)cc2-c2ccccc2)cc1 ; c1ccc(-c2ccc(N(c3ccc(-c4ccccc4-c4ccccc4)cc3)c3ccc4c5ccccc5n(-c5ccccc5)c4c3)cc2)cc1 ; c1ccc(-c2ccc(N(c3cccc(-c4ccccc4-c4ccccc4)c3)c3ccc4c5ccccc5n(-c5ccccc5)c4c3)cc2)cc1 ; c1ccc(-c2ccc(N(c3ccc4c5ccccc5n(-c5ccccc5)c4c3)c3ccccc3-c3ccccc3-c3ccccc3)cc2)cc1]

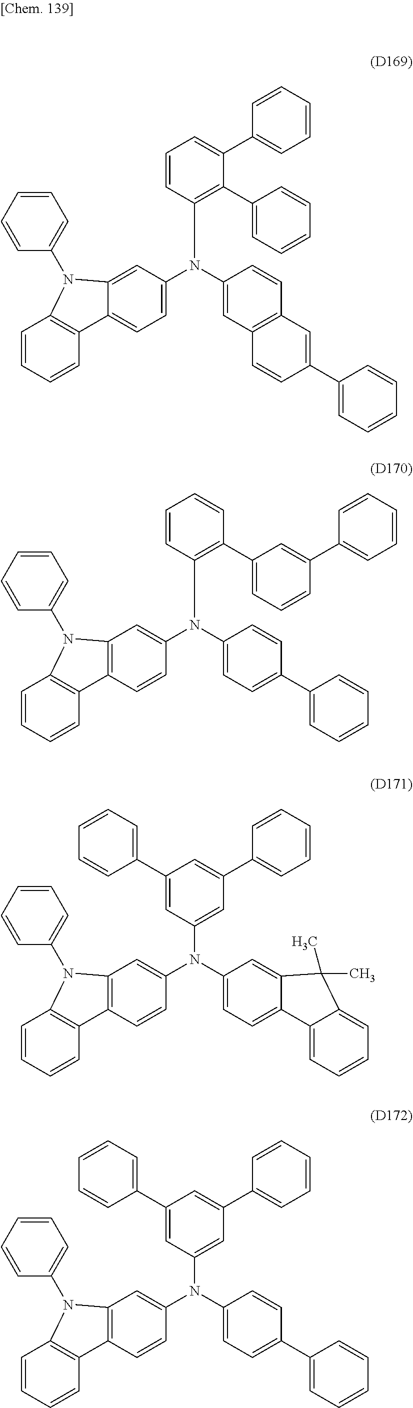 [SMILES: c1ccc(-c2ccc3cc(N(c4ccc5c6ccccc6n(-c6ccccc6)c5c4)c4cccc(-c5ccccc5)c4-c4ccccc4)ccc3c2)cc1 ; c1ccc(-c2ccc(N(c3ccc4c5ccccc5n(-c5ccccc5)c4c3)c3ccccc3-c3cccc(-c4ccccc4)c3)cc2)cc1 ; CC1(C)c2ccccc2-c2ccc(N(c3cc(-c4ccccc4)cc(-c4ccccc4)c3)c3ccc4c5ccccc5n(-c5ccccc5)c4c3)cc21 ; c1ccc(-c2ccc(N(c3cc(-c4ccccc4)cc(-c4ccccc4)c3)c3ccc4c5ccccc5n(-c5ccccc5)c4c3)cc2)cc1]

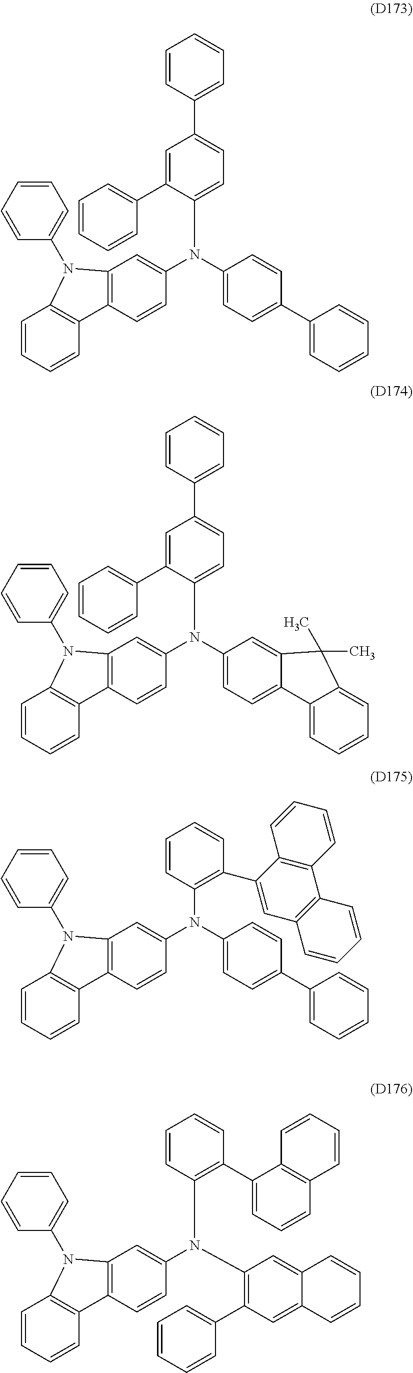 [SMILES: c1ccc(-c2ccc(N(c3ccc4c5ccccc5n(-c5ccccc5)c4c3)c3ccc(-c4ccccc4)cc3-c3ccccc3)cc2)cc1 ; CC1(C)c2ccccc2-c2ccc(N(c3ccc4c5ccccc5n(-c5ccccc5)c4c3)c3ccc(-c4ccccc4)cc3-c3ccccc3)cc21 ; c1ccc(-c2ccc(N(c3ccc4c5ccccc5n(-c5ccccc5)c4c3)c3ccccc3-c3cc4ccccc4c4ccccc34)cc2)cc1 ; c1ccc(-c2cc3ccccc3cc2N(c2ccc3c4ccccc4n(-c4ccccc4)c3c2)c2ccccc2-c2cccc3ccccc23)cc1]

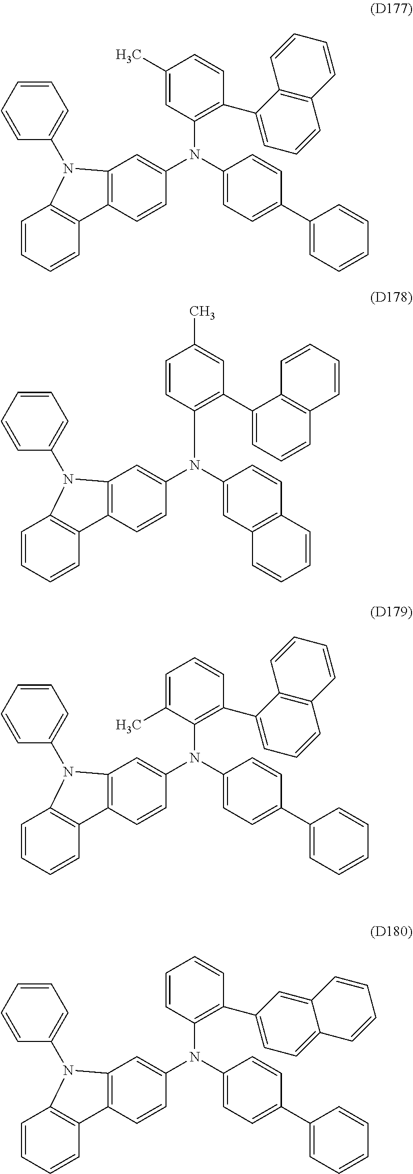 [SMILES: Cc1ccc(-c2cccc3ccccc23)c(N(c2ccc(-c3ccccc3)cc2)c2ccc3c4ccccc4n(-c4ccccc4)c3c2)c1 ; Cc1ccc(N(c2ccc3ccccc3c2)c2ccc3c4ccccc4n(-c4ccccc4)c3c2)c(-c2cccc3ccccc23)c1 ; Cc1cccc(-c2cccc3ccccc23)c1N(c1ccc(-c2ccccc2)cc1)c1ccc2c3ccccc3n(-c3ccccc3)c2c1 ; c1ccc(-c2ccc(N(c3ccc4c5ccccc5n(-c5ccccc5)c4c3)c3ccccc3-c3ccc4ccccc4c3)cc2)cc1]

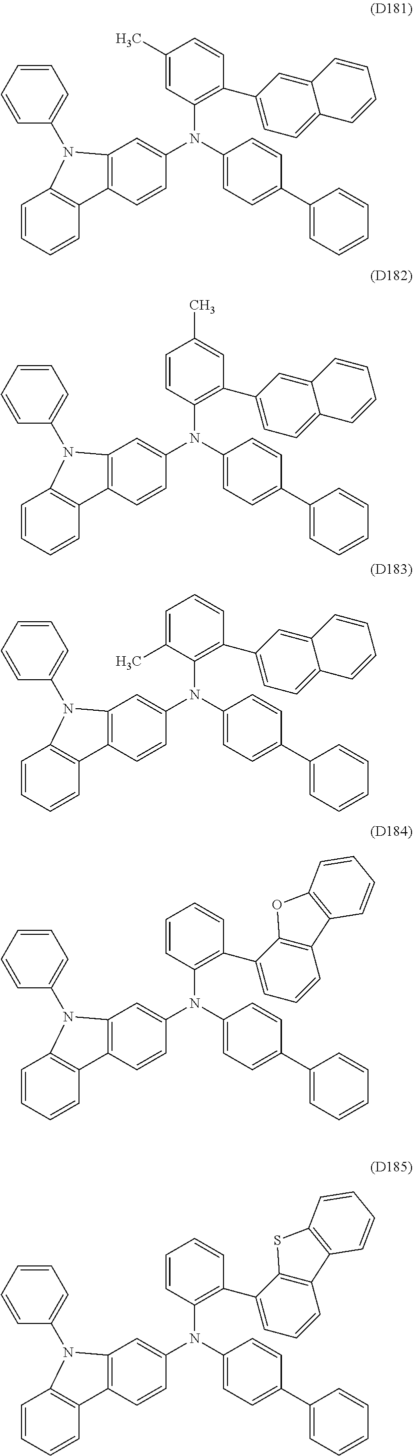 [SMILES: Cc1ccc(-c2ccc3ccccc3c2)c(N(c2ccc(-c3ccccc3)cc2)c2ccc3c4ccccc4n(-c4ccccc4)c3c2)c1 ; Cc1ccc(N(c2ccc(-c3ccccc3)cc2)c2ccc3c4ccccc4n(-c4ccccc4)c3c2)c(-c2ccc3ccccc3c2)c1 ; Cc1cccc(-c2ccc3ccccc3c2)c1N(c1ccc(-c2ccccc2)cc1)c1ccc2c3ccccc3n(-c3ccccc3)c2c1 ; c1ccc(-c2ccc(N(c3ccc4c5ccccc5n(-c5ccccc5)c4c3)c3ccccc3-c3cccc4c3oc3ccccc34)cc2)cc1 ; c1ccc(-c2ccc(N(c3ccc4c5ccccc5n(-c5ccccc5)c4c3)c3ccccc3-c3cccc4c3sc3ccccc34)cc2)cc1]

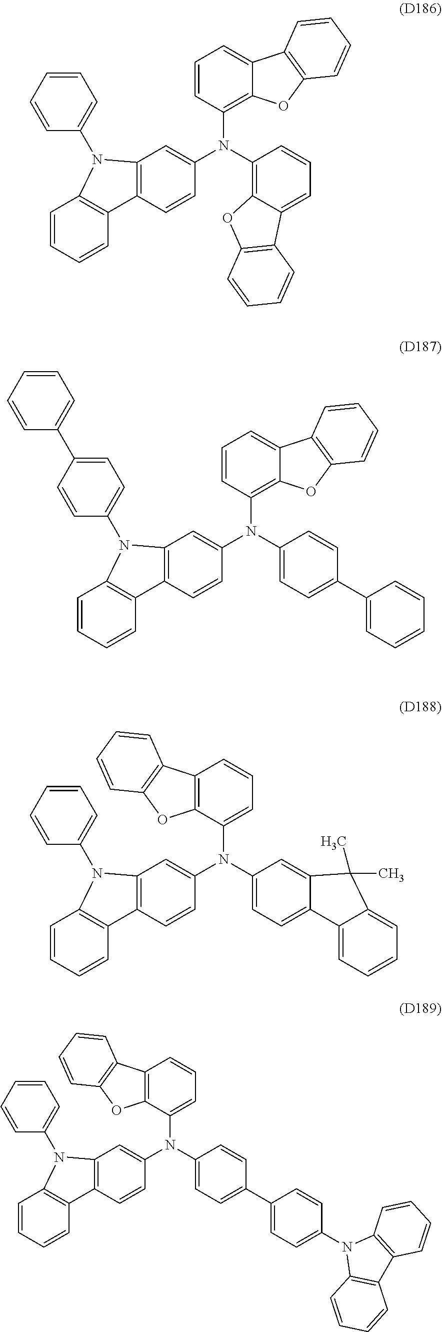 [SMILES: c1ccc(-n2c3ccccc3c3ccc(N(c4cccc5c4oc4ccccc45)c4cccc5c4oc4ccccc45)cc32)cc1 ; c1ccc(-c2ccc(N(c3ccc4c5ccccc5n(-c5ccc(-c6ccccc6)cc5)c4c3)c3cccc4c3oc3ccccc34)cc2)cc1 ; CC1(C)c2ccccc2-c2ccc(N(c3ccc4c5ccccc5n(-c5ccccc5)c4c3)c3cccc4c3oc3ccccc34)cc21 ; c1ccc(-n2c3ccccc3c3ccc(N(c4ccc(-c5ccc(-n6c7ccccc7c7ccccc76)cc5)cc4)c4cccc5c4oc4ccccc45)cc32)cc1]

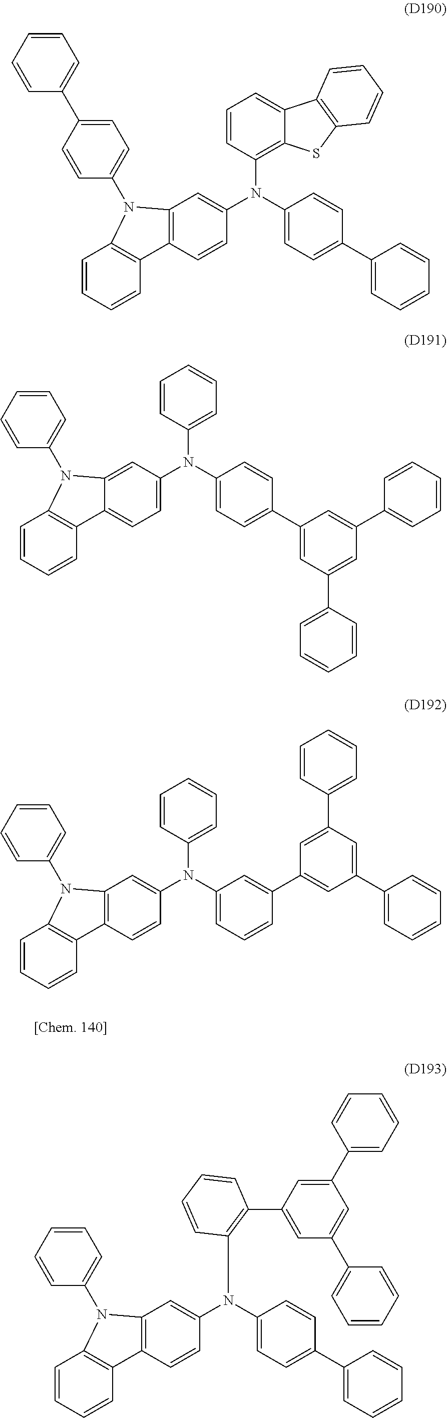 [SMILES: c1ccc(-c2ccc(N(c3ccc4c5ccccc5n(-c5ccc(-c6ccccc6)cc5)c4c3)c3cccc4c3sc3ccccc34)cc2)cc1 ; c1ccc(-c2cc(-c3ccccc3)cc(-c3ccc(N(c4ccccc4)c4ccc5c6ccccc6n(-c6ccccc6)c5c4)cc3)c2)cc1 ; c1ccc(-c2cc(-c3ccccc3)cc(-c3cccc(N(c4ccccc4)c4ccc5c6ccccc6n(-c6ccccc6)c5c4)c3)c2)cc1 ; c1ccc(-c2ccc(N(c3ccc4c5ccccc5n(-c5ccccc5)c4c3)c3ccccc3-c3cc(-c4ccccc4)cc(-c4ccccc4)c3)cc2)cc1]

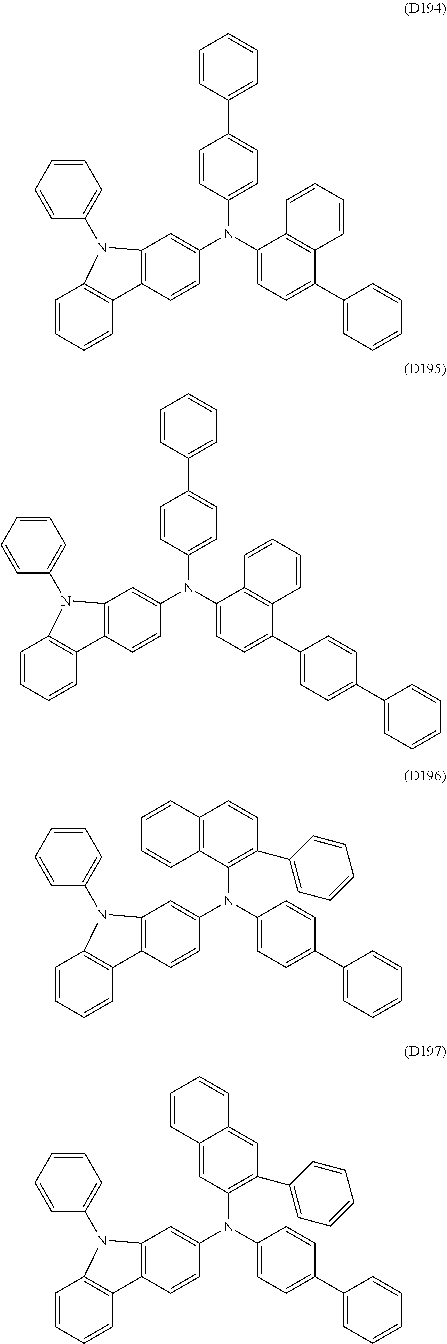 [SMILES: c1ccc(-c2ccc(N(c3ccc4c5ccccc5n(-c5ccccc5)c4c3)c3ccc(-c4ccccc4)c4ccccc34)cc2)cc1 ; c1ccc(-c2ccc(-c3ccc(N(c4ccc(-c5ccccc5)cc4)c4ccc5c6ccccc6n(-c6ccccc6)c5c4)c4ccccc34)cc2)cc1 ; c1ccc(-c2ccc(N(c3ccc4c5ccccc5n(-c5ccccc5)c4c3)c3c(-c4ccccc4)ccc4ccccc34)cc2)cc1 ; c1ccc(-c2ccc(N(c3ccc4c5ccccc5n(-c5ccccc5)c4c3)c3cc4ccccc4cc3-c3ccccc3)cc2)cc1]

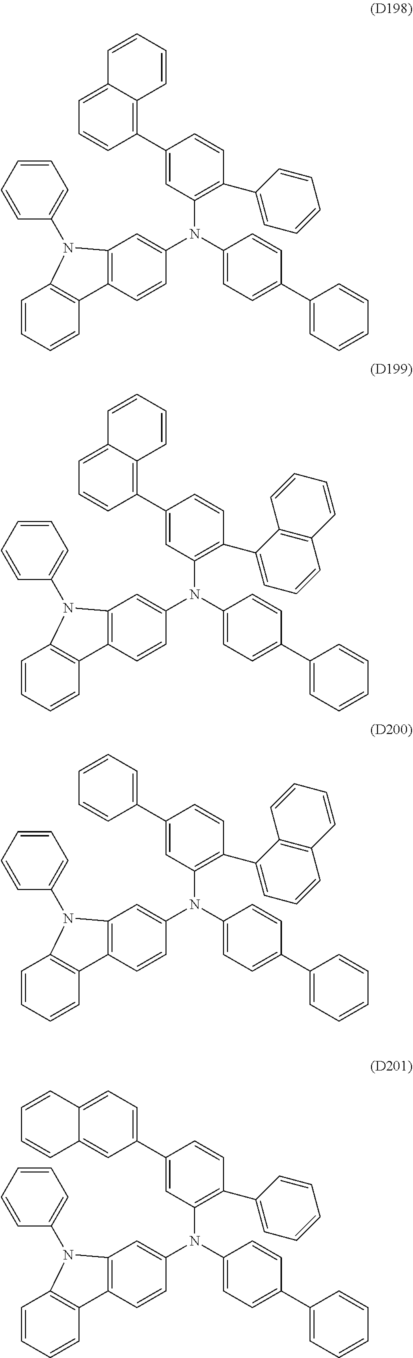 [SMILES: c1ccc(-c2ccc(N(c3ccc4c5ccccc5n(-c5ccccc5)c4c3)c3cc(-c4cccc5ccccc45)ccc3-c3ccccc3)cc2)cc1 ; c1ccc(-c2ccc(N(c3ccc4c5ccccc5n(-c5ccccc5)c4c3)c3cc(-c4cccc5ccccc45)ccc3-c3cccc4ccccc34)cc2)cc1 ; c1ccc(-c2ccc(N(c3ccc4c5ccccc5n(-c5ccccc5)c4c3)c3cc(-c4ccccc4)ccc3-c3cccc4ccccc34)cc2)cc1 ; c1ccc(-c2ccc(N(c3ccc4c5ccccc5n(-c5ccccc5)c4c3)c3cc(-c4ccc5ccccc5c4)ccc3-c3ccccc3)cc2)cc1]

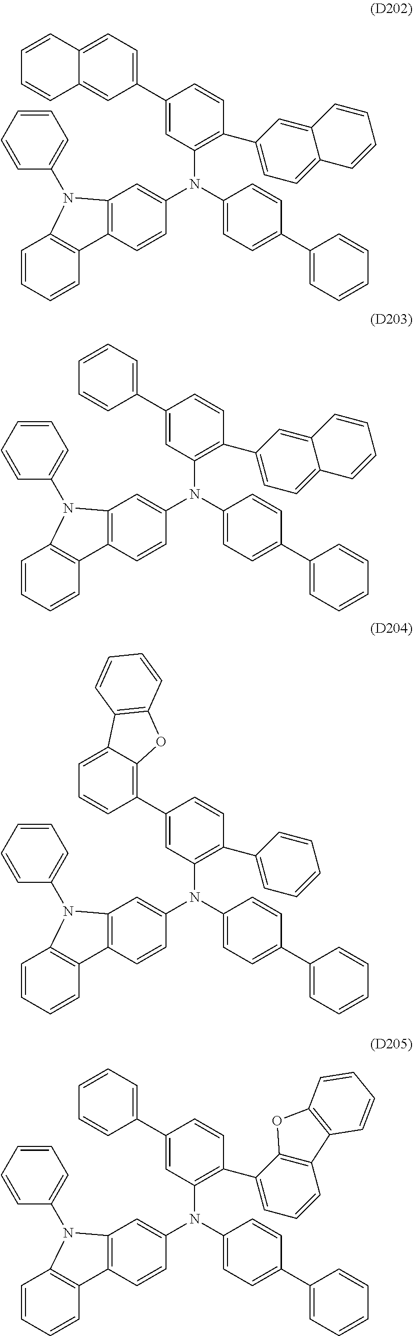 [SMILES: c1ccc(-c2ccc(N(c3ccc4c5ccccc5n(-c5ccccc5)c4c3)c3cc(-c4ccc5ccccc5c4)ccc3-c3ccc4ccccc4c3)cc2)cc1 ; c1ccc(-c2ccc(N(c3ccc4c5ccccc5n(-c5ccccc5)c4c3)c3cc(-c4ccccc4)ccc3-c3ccc4ccccc4c3)cc2)cc1 ; c1ccc(-c2ccc(N(c3ccc4c5ccccc5n(-c5ccccc5)c4c3)c3cc(-c4cccc5c4oc4ccccc45)ccc3-c3ccccc3)cc2)cc1 ; c1ccc(-c2ccc(N(c3ccc4c5ccccc5n(-c5ccccc5)c4c3)c3cc(-c4ccccc4)ccc3-c3cccc4c3oc3ccccc34)cc2)cc1]

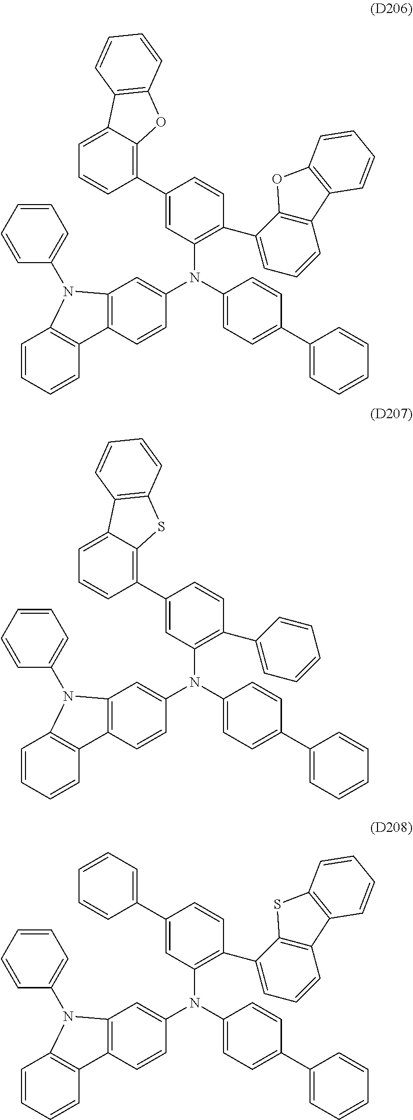 [SMILES: c1ccc(-c2ccc(N(c3ccc4c5ccccc5n(-c5ccccc5)c4c3)c3cc(-c4cccc5c4oc4ccccc45)ccc3-c3cccc4c3oc3ccccc34)cc2)cc1 ; c1ccc(-c2ccc(N(c3ccc4c5ccccc5n(-c5ccccc5)c4c3)c3cc(-c4cccc5c4sc4ccccc45)ccc3-c3ccccc3)cc2)cc1 ; c1ccc(-c2ccc(N(c3ccc4c5ccccc5n(-c5ccccc5)c4c3)c3cc(-c4ccccc4)ccc3-c3cccc4c3sc3ccccc34)cc2)cc1]

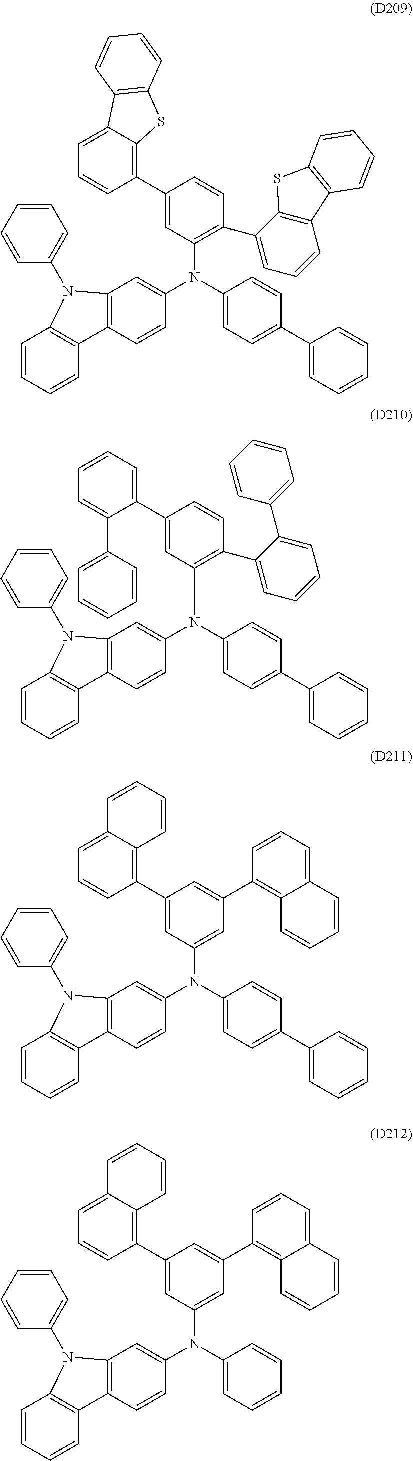 [SMILES: c1ccc(-c2ccc(N(c3ccc4c5ccccc5n(-c5ccccc5)c4c3)c3cc(-c4cccc5c4sc4ccccc45)ccc3-c3cccc4c3sc3ccccc34)cc2)cc1 ; c1ccc(-c2ccc(N(c3ccc4c5ccccc5n(-c5ccccc5)c4c3)c3cc(-c4ccccc4-c4ccccc4)ccc3-c3ccccc3-c3ccccc3)cc2)cc1 ; c1ccc(-c2ccc(N(c3cc(-c4cccc5ccccc45)cc(-c4cccc5ccccc45)c3)c3ccc4c5ccccc5n(-c5ccccc5)c4c3)cc2)cc1 ; c1ccc(N(c2cc(-c3cccc4ccccc34)cc(-c3cccc4ccccc34)c2)c2ccc3c4ccccc4n(-c4ccccc4)c3c2)cc1]

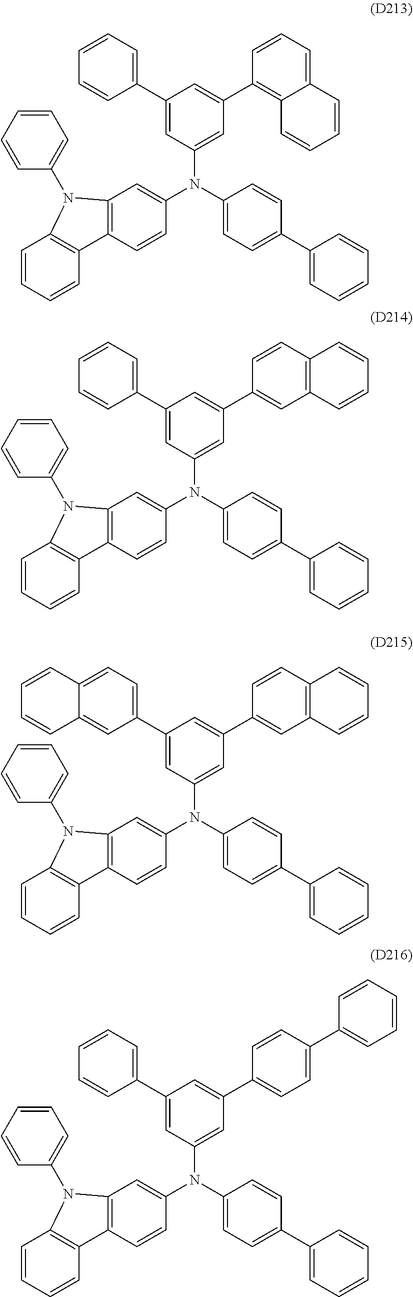

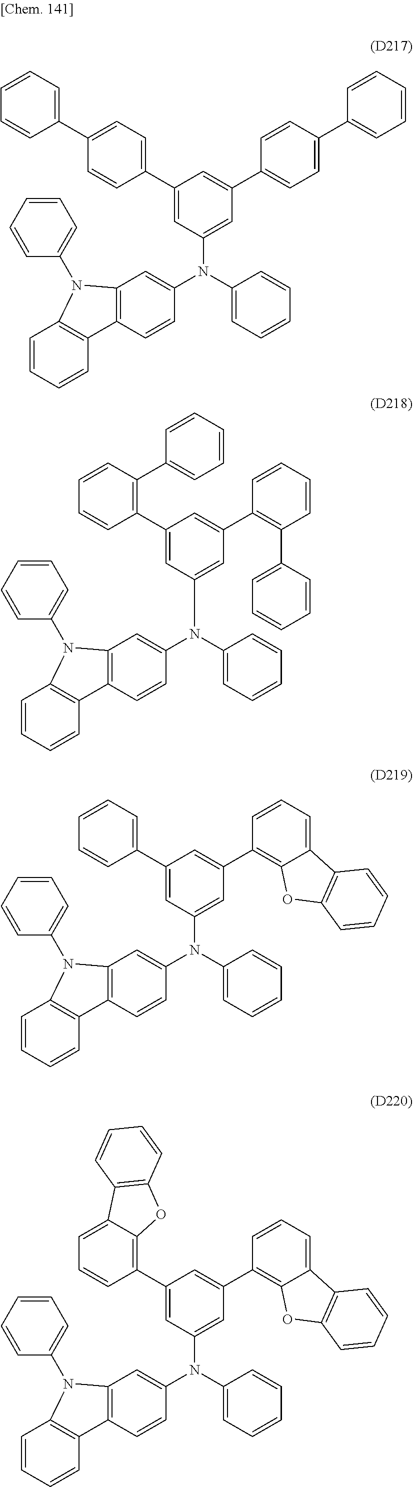 [SMILES: c1ccc(-c2ccc(-c3cc(-c4ccc(-c5ccccc5)cc4)cc(N(c4ccccc4)c4ccc5c6ccccc6n(-c6ccccc6)c5c4)c3)cc2)cc1 ; c1ccc(-c2ccccc2-c2cc(-c3ccccc3-c3ccccc3)cc(N(c3ccccc3)c3ccc4c5ccccc5n(-c5ccccc5)c4c3)c2)cc1 ; c1ccc(-c2cc(-c3cccc4c3oc3ccccc34)cc(N(c3ccccc3)c3ccc4c5ccccc5n(-c5ccccc5)c4c3)c2)cc1 ; c1ccc(N(c2cc(-c3cccc4c3oc3ccccc34)cc(-c3cccc4c3oc3ccccc34)c2)c2ccc3c4ccccc4n(-c4ccccc4)c3c2)cc1]

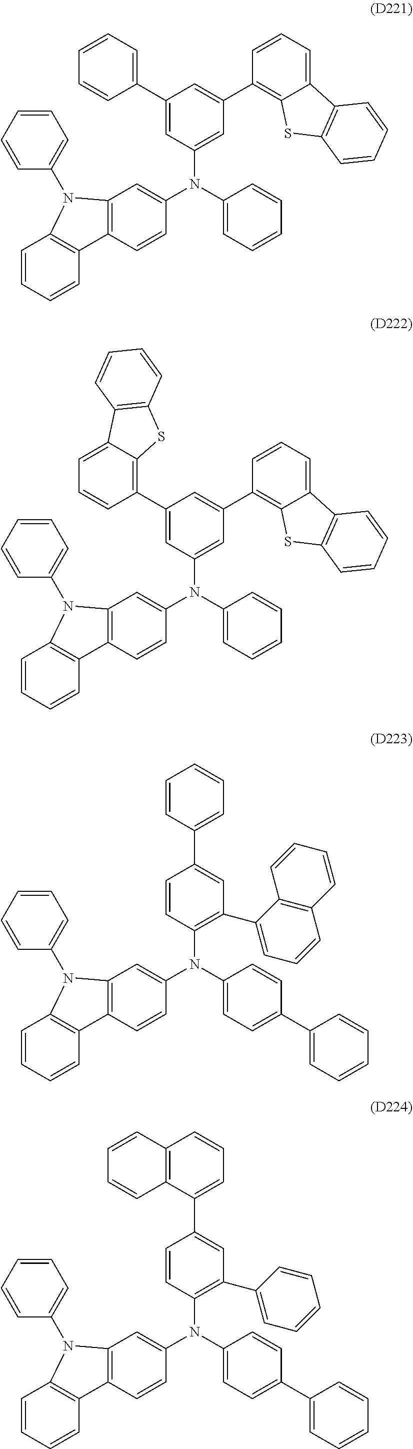 [SMILES: c1ccc(-c2cc(-c3cccc4c3sc3ccccc34)cc(N(c3ccccc3)c3ccc4c5ccccc5n(-c5ccccc5)c4c3)c2)cc1 ; c1ccc(N(c2cc(-c3cccc4c3sc3ccccc34)cc(-c3cccc4c3sc3ccccc34)c2)c2ccc3c4ccccc4n(-c4ccccc4)c3c2)cc1 ; c1ccc(-c2ccc(N(c3ccc4c5ccccc5n(-c5ccccc5)c4c3)c3ccc(-c4ccccc4)cc3-c3cccc4ccccc34)cc2)cc1 ; c1ccc(-c2ccc(N(c3ccc4c5ccccc5n(-c5ccccc5)c4c3)c3ccc(-c4cccc5ccccc45)cc3-c3ccccc3)cc2)cc1]

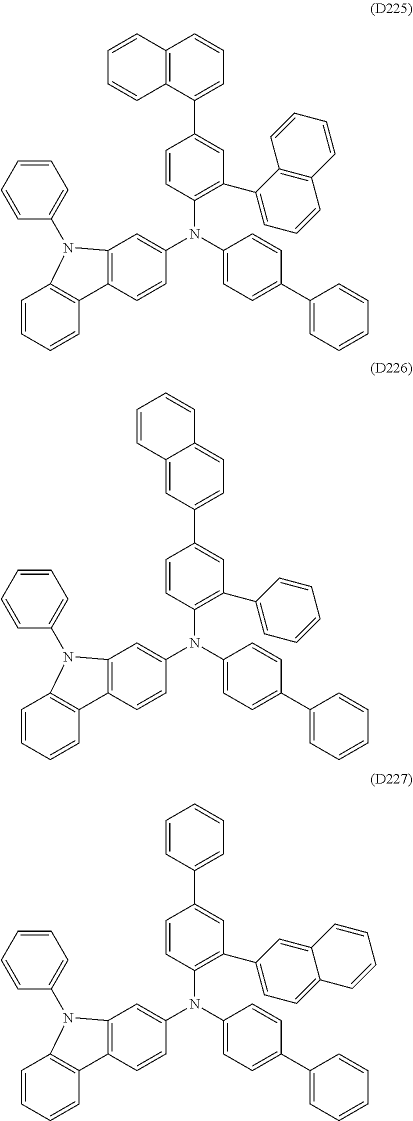 [SMILES: c1ccc(-c2ccc(N(c3ccc4c5ccccc5n(-c5ccccc5)c4c3)c3ccc(-c4cccc5ccccc45)cc3-c3cccc4ccccc34)cc2)cc1 ; c1ccc(-c2ccc(N(c3ccc4c5ccccc5n(-c5ccccc5)c4c3)c3ccc(-c4ccc5ccccc5c4)cc3-c3ccccc3)cc2)cc1 ; c1ccc(-c2ccc(N(c3ccc4c5ccccc5n(-c5ccccc5)c4c3)c3ccc(-c4ccccc4)cc3-c3ccc4ccccc4c3)cc2)cc1]

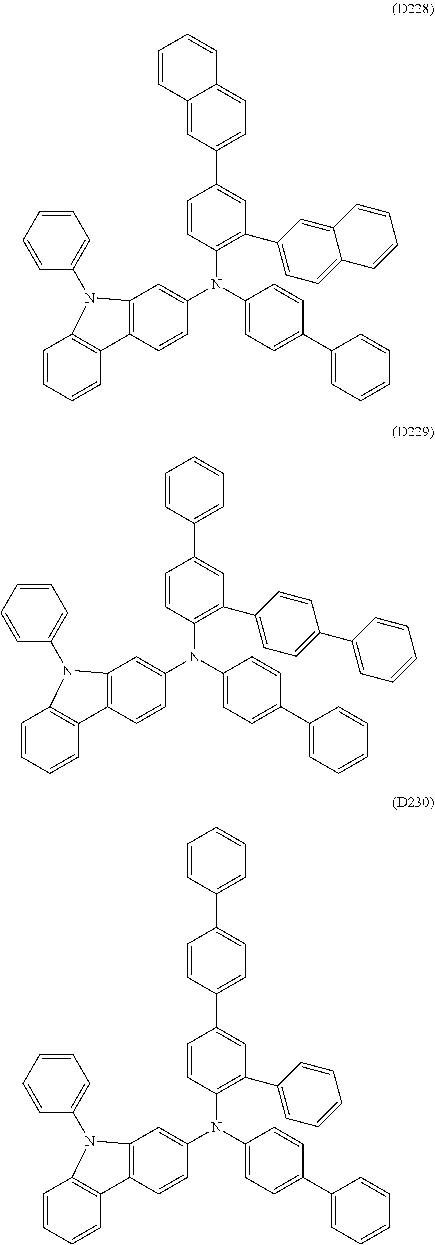 [SMILES: c1ccc(-c2ccc(N(c3ccc4c5ccccc5n(-c5ccccc5)c4c3)c3ccc(-c4ccc5ccccc5c4)cc3-c3ccc4ccccc4c3)cc2)cc1 ; c1ccc(-c2ccc(-c3cc(-c4ccccc4)ccc3N(c3ccc(-c4ccccc4)cc3)c3ccc4c5ccccc5n(-c5ccccc5)c4c3)cc2)cc1 ; c1ccc(-c2ccc(-c3ccc(N(c4ccc(-c5ccccc5)cc4)c4ccc5c6ccccc6n(-c6ccccc6)c5c4)c(-c4ccccc4)c3)cc2)cc1]

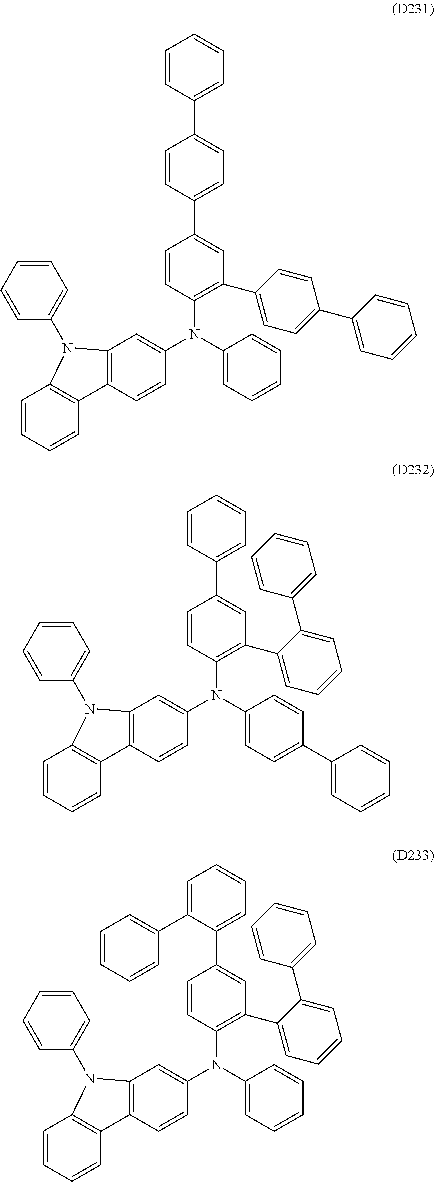 [SMILES: c1ccc(-c2ccc(-c3ccc(N(c4ccccc4)c4ccc5c6ccccc6n(-c6ccccc6)c5c4)c(-c4ccc(-c5ccccc5)cc4)c3)cc2)cc1 ; c1ccc(-c2ccc(N(c3ccc4c5ccccc5n(-c5ccccc5)c4c3)c3ccc(-c4ccccc4)cc3-c3ccccc3-c3ccccc3)cc2)cc1 ; c1ccc(-c2ccccc2-c2ccc(N(c3ccccc3)c3ccc4c5ccccc5n(-c5ccccc5)c4c3)c(-c3ccccc3-c3ccccc3)c2)cc1]

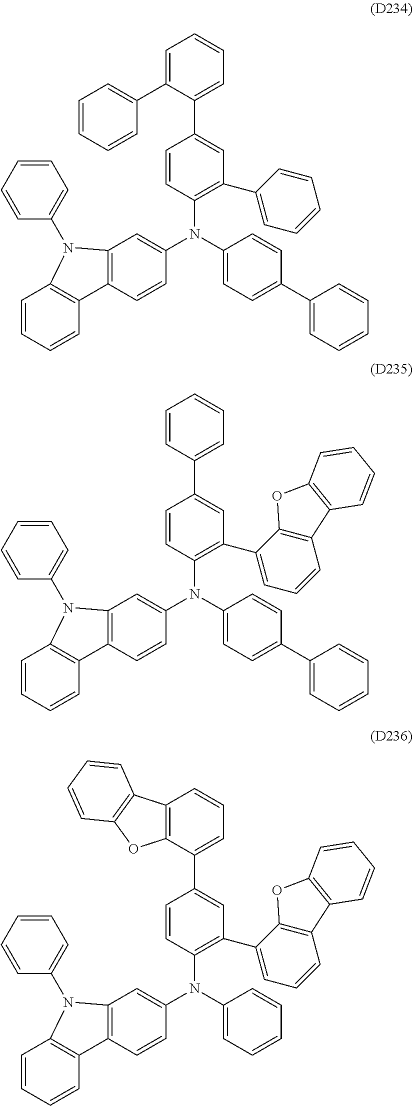 [SMILES: c1ccc(-c2ccc(N(c3ccc4c5ccccc5n(-c5ccccc5)c4c3)c3ccc(-c4ccccc4-c4ccccc4)cc3-c3ccccc3)cc2)cc1 ; c1ccc(-c2ccc(N(c3ccc4c5ccccc5n(-c5ccccc5)c4c3)c3ccc(-c4ccccc4)cc3-c3cccc4c3oc3ccccc34)cc2)cc1 ; c1ccc(N(c2ccc3c4ccccc4n(-c4ccccc4)c3c2)c2ccc(-c3cccc4c3oc3ccccc34)cc2-c2cccc3c2oc2ccccc23)cc1]

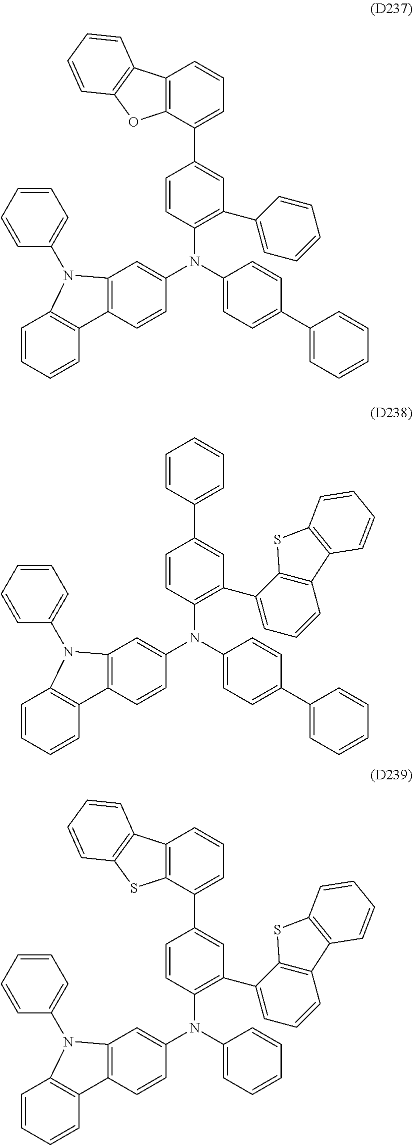 [SMILES: c1ccc(-c2ccc(N(c3ccc4c5ccccc5n(-c5ccccc5)c4c3)c3ccc(-c4cccc5c4oc4ccccc45)cc3-c3ccccc3)cc2)cc1 ; c1ccc(-c2ccc(N(c3ccc4c5ccccc5n(-c5ccccc5)c4c3)c3ccc(-c4ccccc4)cc3-c3cccc4c3sc3ccccc34)cc2)cc1 ; c1ccc(N(c2ccc3c4ccccc4n(-c4ccccc4)c3c2)c2ccc(-c3cccc4c3sc3ccccc34)cc2-c2cccc3c2sc2ccccc23)cc1]

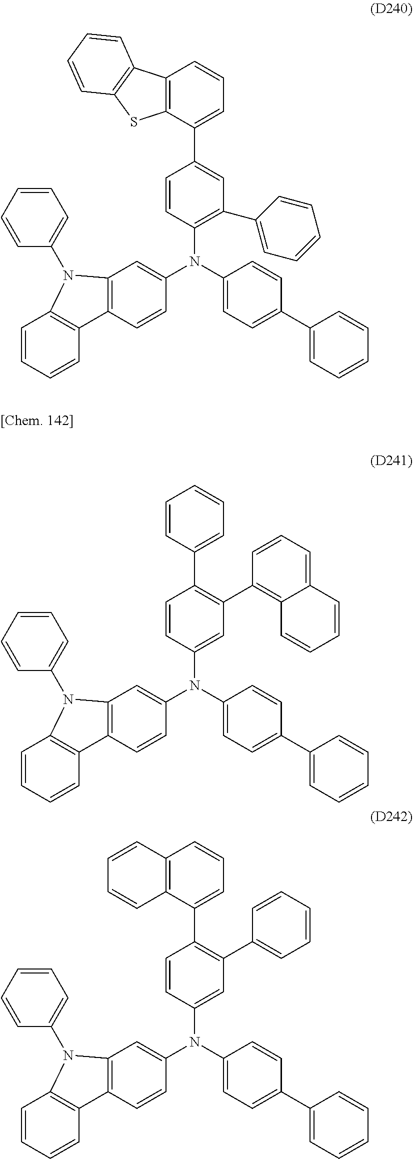 [SMILES: c1ccc(-c2ccc(N(c3ccc4c5ccccc5n(-c5ccccc5)c4c3)c3ccc(-c4cccc5c4sc4ccccc45)cc3-c3ccccc3)cc2)cc1 ; c1ccc(-c2ccc(N(c3ccc(-c4ccccc4)c(-c4cccc5ccccc45)c3)c3ccc4c5ccccc5n(-c5ccccc5)c4c3)cc2)cc1 ; c1ccc(-c2ccc(N(c3ccc(-c4cccc5ccccc45)c(-c4ccccc4)c3)c3ccc4c5ccccc5n(-c5ccccc5)c4c3)cc2)cc1]

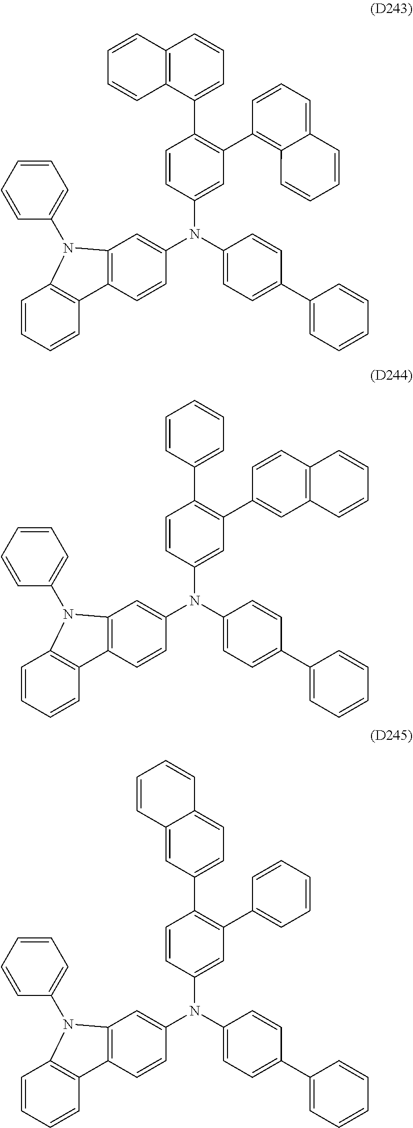 [SMILES: c1ccc(-c2ccc(N(c3ccc(-c4cccc5ccccc45)c(-c4cccc5ccccc45)c3)c3ccc4c5ccccc5n(-c5ccccc5)c4c3)cc2)cc1 ; c1ccc(-c2ccc(N(c3ccc(-c4ccccc4)c(-c4ccc5ccccc5c4)c3)c3ccc4c5ccccc5n(-c5ccccc5)c4c3)cc2)cc1 ; c1ccc(-c2ccc(N(c3ccc(-c4ccc5ccccc5c4)c(-c4ccccc4)c3)c3ccc4c5ccccc5n(-c5ccccc5)c4c3)cc2)cc1]

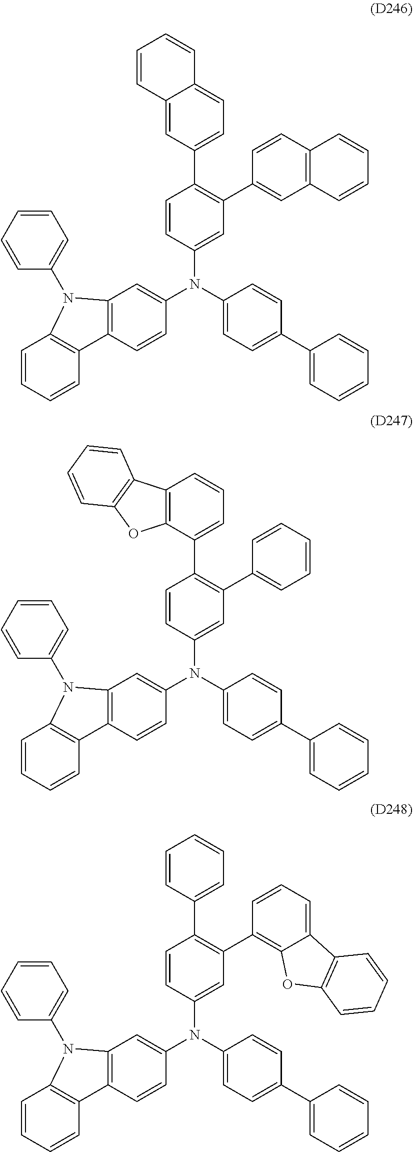 [SMILES: c1ccc(-c2ccc(N(c3ccc(-c4ccc5ccccc5c4)c(-c4ccc5ccccc5c4)c3)c3ccc4c5ccccc5n(-c5ccccc5)c4c3)cc2)cc1 ; c1ccc(-c2ccc(N(c3ccc(-c4cccc5c4oc4ccccc45)c(-c4ccccc4)c3)c3ccc4c5ccccc5n(-c5ccccc5)c4c3)cc2)cc1 ; c1ccc(-c2ccc(N(c3ccc(-c4ccccc4)c(-c4cccc5c4oc4ccccc45)c3)c3ccc4c5ccccc5n(-c5ccccc5)c4c3)cc2)cc1]

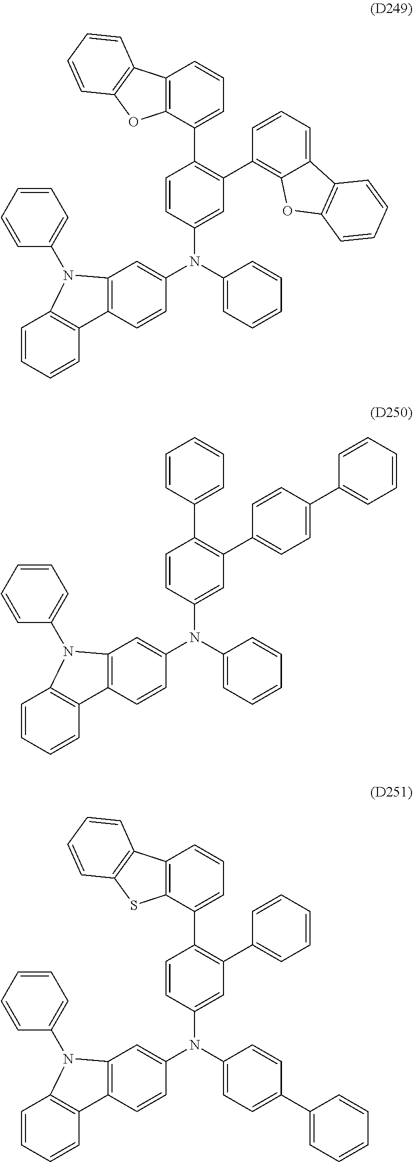 [SMILES: c1ccc(N(c2ccc(-c3cccc4c3oc3ccccc34)c(-c3cccc4c3oc3ccccc34)c2)c2ccc3c4ccccc4n(-c4ccccc4)c3c2)cc1 ; c1ccc(-c2ccc(-c3cc(N(c4ccccc4)c4ccc5c6ccccc6n(-c6ccccc6)c5c4)ccc3-c3ccccc3)cc2)cc1 ; c1ccc(-c2ccc(N(c3ccc(-c4cccc5c4sc4ccccc45)c(-c4ccccc4)c3)c3ccc4c5ccccc5n(-c5ccccc5)c4c3)cc2)cc1]

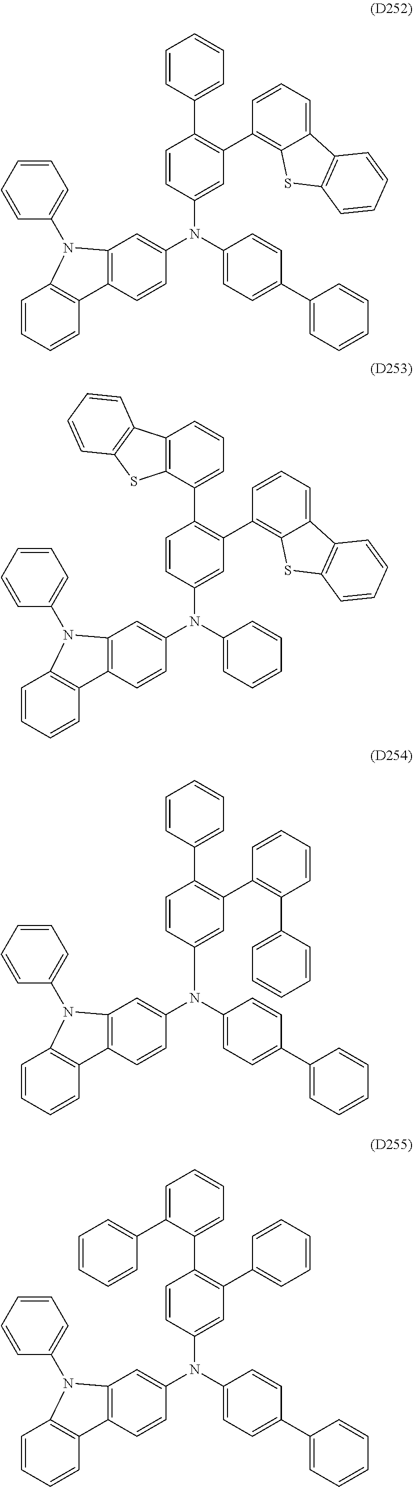 [SMILES: c1ccc(-c2ccc(N(c3ccc(-c4ccccc4)c(-c4cccc5c4sc4ccccc45)c3)c3ccc4c5ccccc5n(-c5ccccc5)c4c3)cc2)cc1 ; c1ccc(N(c2ccc(-c3cccc4c3sc3ccccc34)c(-c3cccc4c3sc3ccccc34)c2)c2ccc3c4ccccc4n(-c4ccccc4)c3c2)cc1 ; c1ccc(-c2ccc(N(c3ccc(-c4ccccc4)c(-c4ccccc4-c4ccccc4)c3)c3ccc4c5ccccc5n(-c5ccccc5)c4c3)cc2)cc1 ; c1ccc(-c2ccc(N(c3ccc(-c4ccccc4-c4ccccc4)c(-c4ccccc4)c3)c3ccc4c5ccccc5n(-c5ccccc5)c4c3)cc2)cc1]

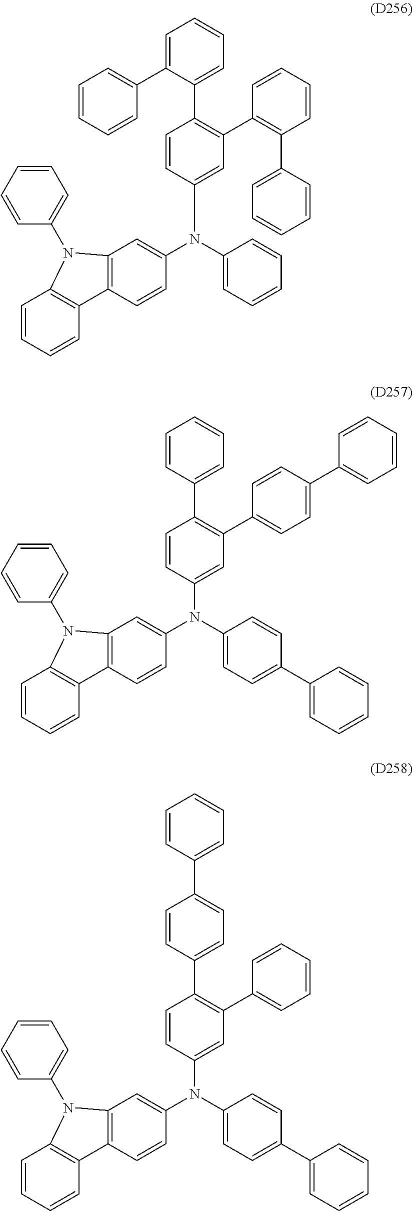 [SMILES: c1ccc(-c2ccccc2-c2ccc(N(c3ccccc3)c3ccc4c5ccccc5n(-c5ccccc5)c4c3)cc2-c2ccccc2-c2ccccc2)cc1 ; c1ccc(-c2ccc(-c3cc(N(c4ccc(-c5ccccc5)cc4)c4ccc5c6ccccc6n(-c6ccccc6)c5c4)ccc3-c3ccccc3)cc2)cc1 ; c1ccc(-c2ccc(-c3ccc(N(c4ccc(-c5ccccc5)cc4)c4ccc5c6ccccc6n(-c6ccccc6)c5c4)cc3-c3ccccc3)cc2)cc1]

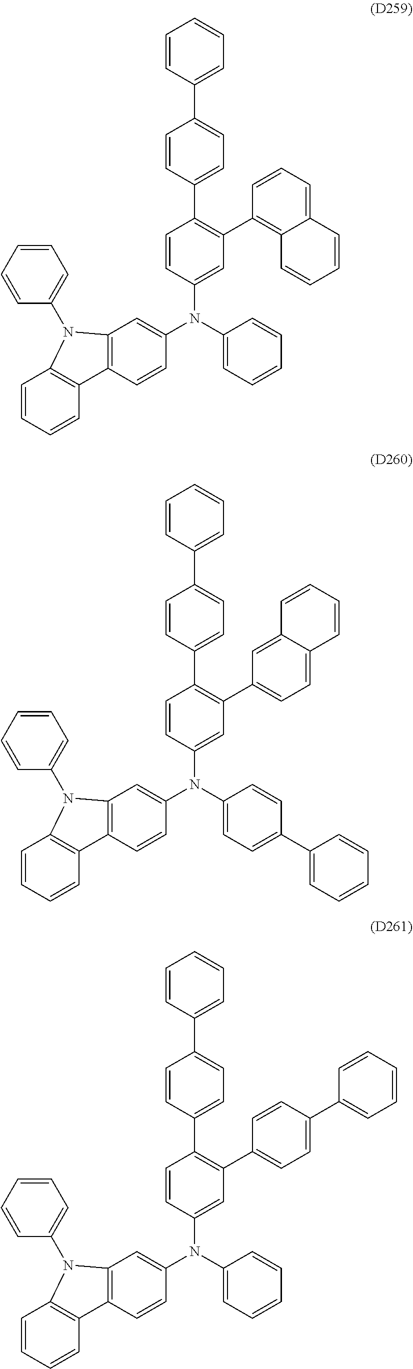 [SMILES: c1ccc(-c2ccc(-c3ccc(N(c4ccccc4)c4ccc5c6ccccc6n(-c6ccccc6)c5c4)cc3-c3cccc4ccccc34)cc2)cc1 ; c1ccc(-c2ccc(-c3ccc(N(c4ccc(-c5ccccc5)cc4)c4ccc5c6ccccc6n(-c6ccccc6)c5c4)cc3-c3ccc4ccccc4c3)cc2)cc1 ; c1ccc(-c2ccc(-c3ccc(N(c4ccccc4)c4ccc5c6ccccc6n(-c6ccccc6)c5c4)cc3-c3ccc(-c4ccccc4)cc3)cc2)cc1]

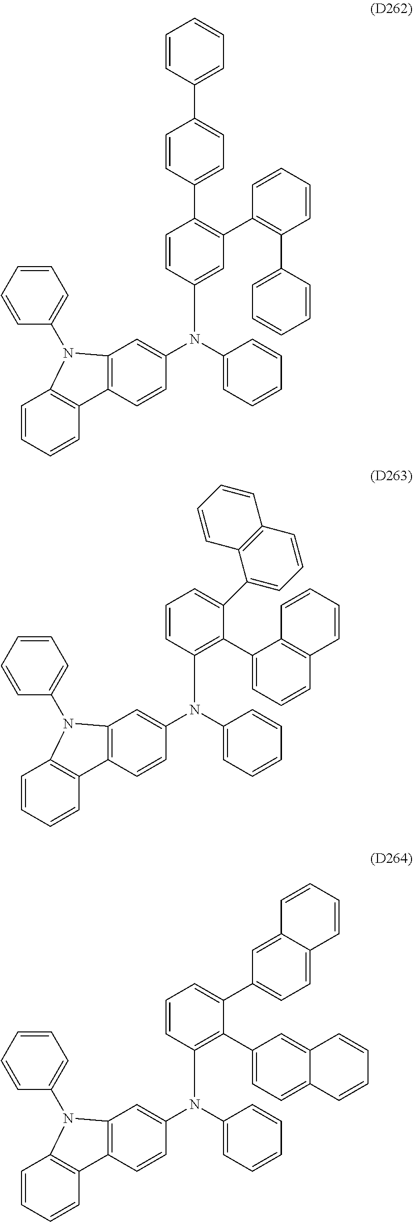 [SMILES: c1ccc(-c2ccc(-c3ccc(N(c4ccccc4)c4ccc5c6ccccc6n(-c6ccccc6)c5c4)cc3-c3ccccc3-c3ccccc3)cc2)cc1 ; c1ccc(N(c2ccc3c4ccccc4n(-c4ccccc4)c3c2)c2cccc(-c3cccc4ccccc34)c2-c2cccc3ccccc23)cc1 ; c1ccc(N(c2ccc3c4ccccc4n(-c4ccccc4)c3c2)c2cccc(-c3ccc4ccccc4c3)c2-c2ccc3ccccc3c2)cc1]

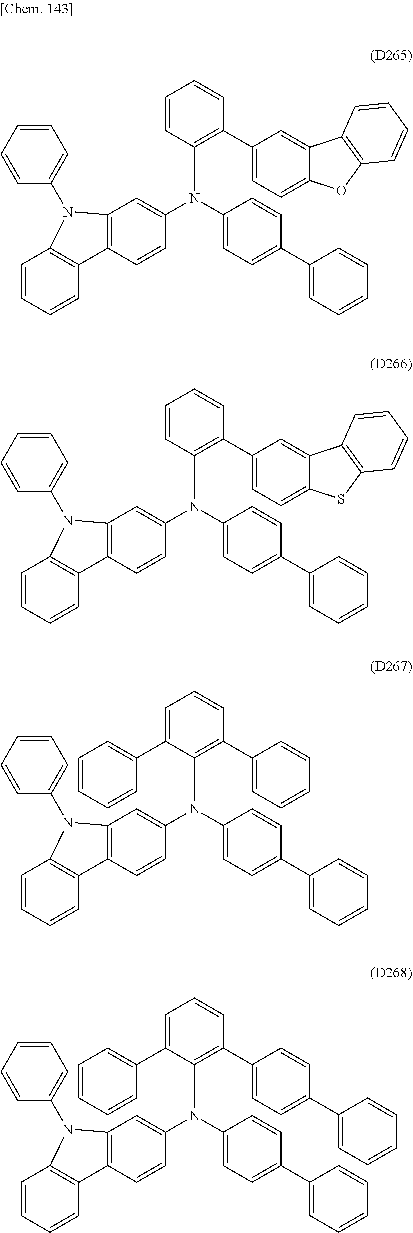 [SMILES: c1ccc(-c2ccc(N(c3ccc4c5ccccc5n(-c5ccccc5)c4c3)c3ccccc3-c3ccc4oc5ccccc5c4c3)cc2)cc1 ; c1ccc(-c2ccc(N(c3ccc4c5ccccc5n(-c5ccccc5)c4c3)c3ccccc3-c3ccc4sc5ccccc5c4c3)cc2)cc1 ; c1ccc(-c2ccc(N(c3ccc4c5ccccc5n(-c5ccccc5)c4c3)c3c(-c4ccccc4)cccc3-c3ccccc3)cc2)cc1 ; c1ccc(-c2ccc(-c3cccc(-c4ccccc4)c3N(c3ccc(-c4ccccc4)cc3)c3ccc4c5ccccc5n(-c5ccccc5)c4c3)cc2)cc1]

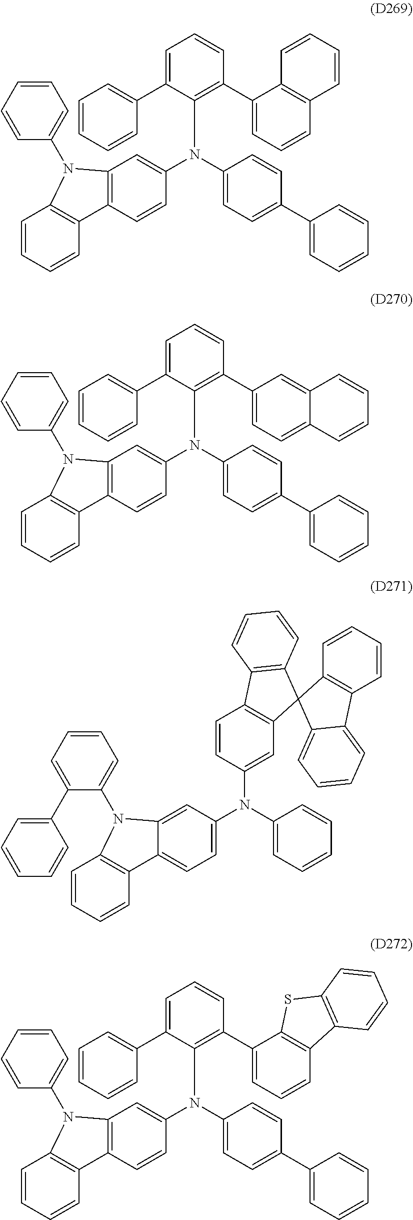 [SMILES: c1ccc(-c2ccc(N(c3ccc4c5ccccc5n(-c5ccccc5)c4c3)c3c(-c4ccccc4)cccc3-c3cccc4ccccc34)cc2)cc1 ; c1ccc(-c2ccc(N(c3ccc4c5ccccc5n(-c5ccccc5)c4c3)c3c(-c4ccccc4)cccc3-c3ccc4ccccc4c3)cc2)cc1 ; c1ccc(-c2ccccc2-n2c3ccccc3c3ccc(N(c4ccccc4)c4ccc5c(c4)C4(c6ccccc6-c6ccccc64)c4ccccc4-5)cc32)cc1 ; c1ccc(-c2ccc(N(c3ccc4c5ccccc5n(-c5ccccc5)c4c3)c3c(-c4ccccc4)cccc3-c3cccc4c3sc3ccccc34)cc2)cc1]

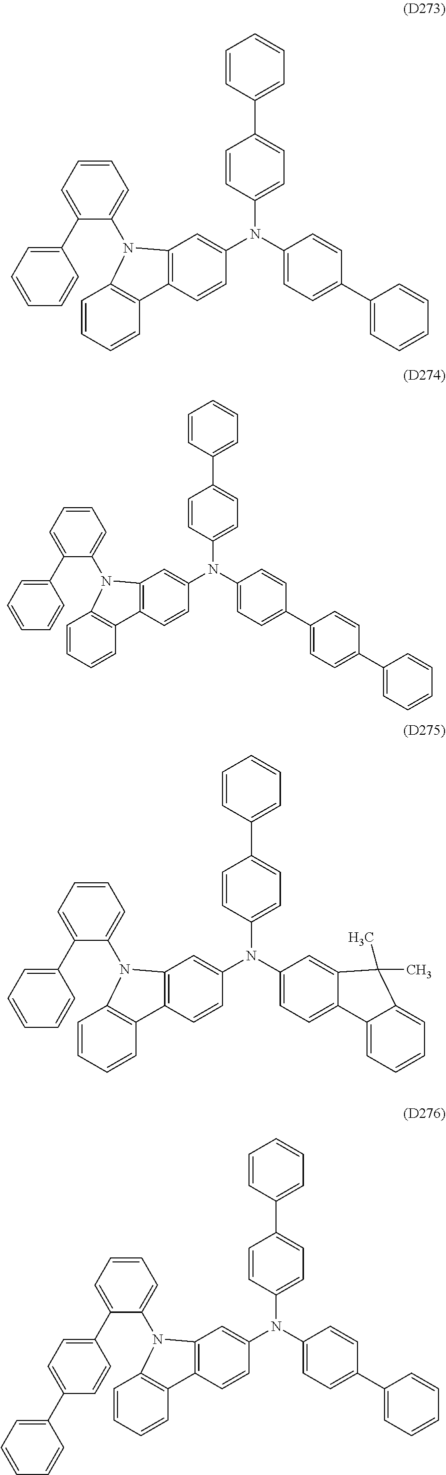 [SMILES: c1ccc(-c2ccc(N(c3ccc(-c4ccccc4)cc3)c3ccc4c5ccccc5n(-c5ccccc5-c5ccccc5)c4c3)cc2)cc1 ; c1ccc(-c2ccc(-c3ccc(N(c4ccc(-c5ccccc5)cc4)c4ccc5c6ccccc6n(-c6ccccc6-c6ccccc6)c5c4)cc3)cc2)cc1 ; CC1(C)c2ccccc2-c2ccc(N(c3ccc(-c4ccccc4)cc3)c3ccc4c5ccccc5n(-c5ccccc5-c5ccccc5)c4c3)cc21 ; c1ccc(-c2ccc(-c3ccccc3-n3c4ccccc4c4ccc(N(c5ccc(-c6ccccc6)cc5)c5ccc(-c6ccccc6)cc5)cc43)cc2)cc1]

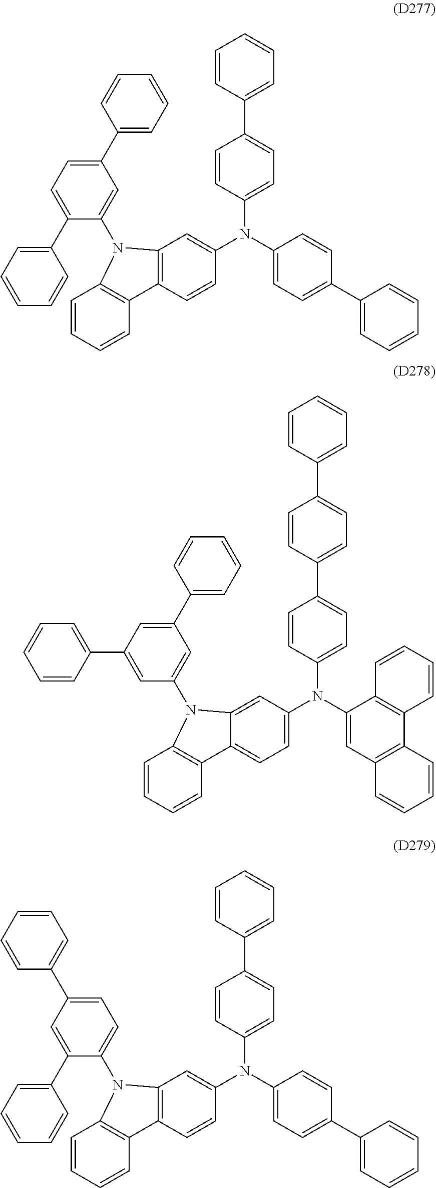 [SMILES: c1ccc(-c2ccc(N(c3ccc(-c4ccccc4)cc3)c3ccc4c5ccccc5n(-c5cc(-c6ccccc6)ccc5-c5ccccc5)c4c3)cc2)cc1 ; c1ccc(-c2ccc(-c3ccc(N(c4ccc5c6ccccc6n(-c6cc(-c7ccccc7)cc(-c7ccccc7)c6)c5c4)c4cc5ccccc5c5ccccc45)cc3)cc2)cc1 ; c1ccc(-c2ccc(N(c3ccc(-c4ccccc4)cc3)c3ccc4c5ccccc5n(-c5ccc(-c6ccccc6)cc5-c5ccccc5)c4c3)cc2)cc1]

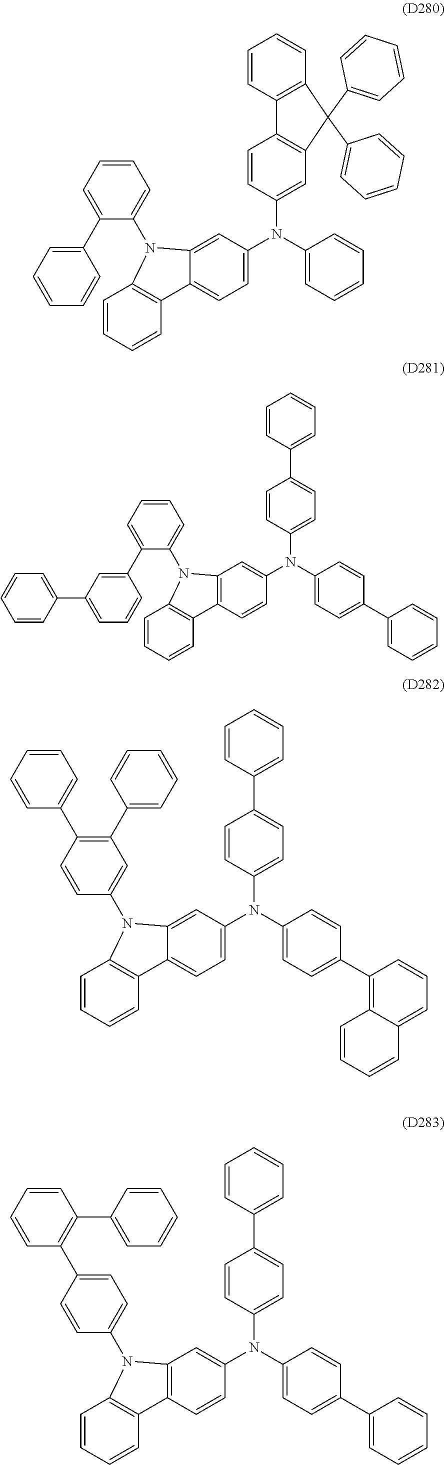 [SMILES: c1ccc(-c2ccccc2-n2c3ccccc3c3ccc(N(c4ccccc4)c4ccc5c(c4)C(c4ccccc4)(c4ccccc4)c4ccccc4-5)cc32)cc1 ; c1ccc(-c2ccc(N(c3ccc(-c4ccccc4)cc3)c3ccc4c5ccccc5n(-c5ccccc5-c5cccc(-c6ccccc6)c5)c4c3)cc2)cc1 ; c1ccc(-c2ccc(N(c3ccc(-c4cccc5ccccc45)cc3)c3ccc4c5ccccc5n(-c5ccc(-c6ccccc6)c(-c6ccccc6)c5)c4c3)cc2)cc1 ; c1ccc(-c2ccc(N(c3ccc(-c4ccccc4)cc3)c3ccc4c5ccccc5n(-c5ccc(-c6ccccc6-c6ccccc6)cc5)c4c3)cc2)cc1]

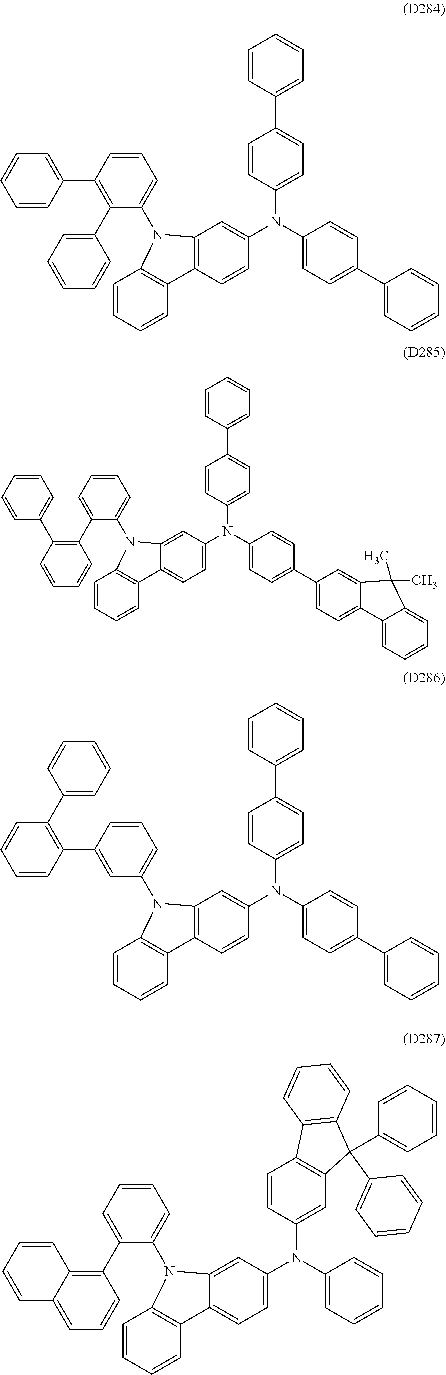 [SMILES: c1ccc(-c2ccc(N(c3ccc(-c4ccccc4)cc3)c3ccc4c5ccccc5n(-c5cccc(-c6ccccc6)c5-c5ccccc5)c4c3)cc2)cc1 ; CC1(C)c2ccccc2-c2ccc(-c3ccc(N(c4ccc(-c5ccccc5)cc4)c4ccc5c6ccccc6n(-c6ccccc6-c6ccccc6-c6ccccc6)c5c4)cc3)cc21 ; c1ccc(-c2ccc(N(c3ccc(-c4ccccc4)cc3)c3ccc4c5ccccc5n(-c5cccc(-c6ccccc6-c6ccccc6)c5)c4c3)cc2)cc1 ; c1ccc(N(c2ccc3c(c2)C(c2ccccc2)(c2ccccc2)c2ccccc2-3)c2ccc3c4ccccc4n(-c4ccccc4-c4cccc5ccccc45)c3c2)cc1]

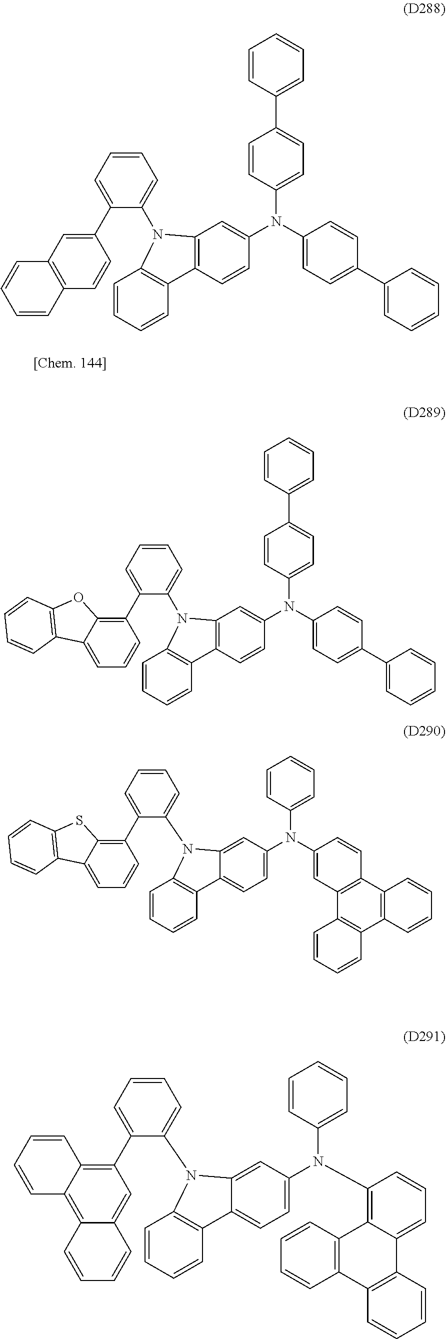 [SMILES: c1ccc(-c2ccc(N(c3ccc(-c4ccccc4)cc3)c3ccc4c5ccccc5n(-c5ccccc5-c5ccc6ccccc6c5)c4c3)cc2)cc1 ; c1ccc(-c2ccc(N(c3ccc(-c4ccccc4)cc3)c3ccc4c5ccccc5n(-c5ccccc5-c5cccc6c5oc5ccccc56)c4c3)cc2)cc1 ; c1ccc(N(c2ccc3c4ccccc4c4ccccc4c3c2)c2ccc3c4ccccc4n(-c4ccccc4-c4cccc5c4sc4ccccc45)c3c2)cc1 ; c1ccc(N(c2ccc3c4ccccc4n(-c4ccccc4-c4cc5ccccc5c5ccccc45)c3c2)c2cccc3c4ccccc4c4ccccc4c23)cc1]

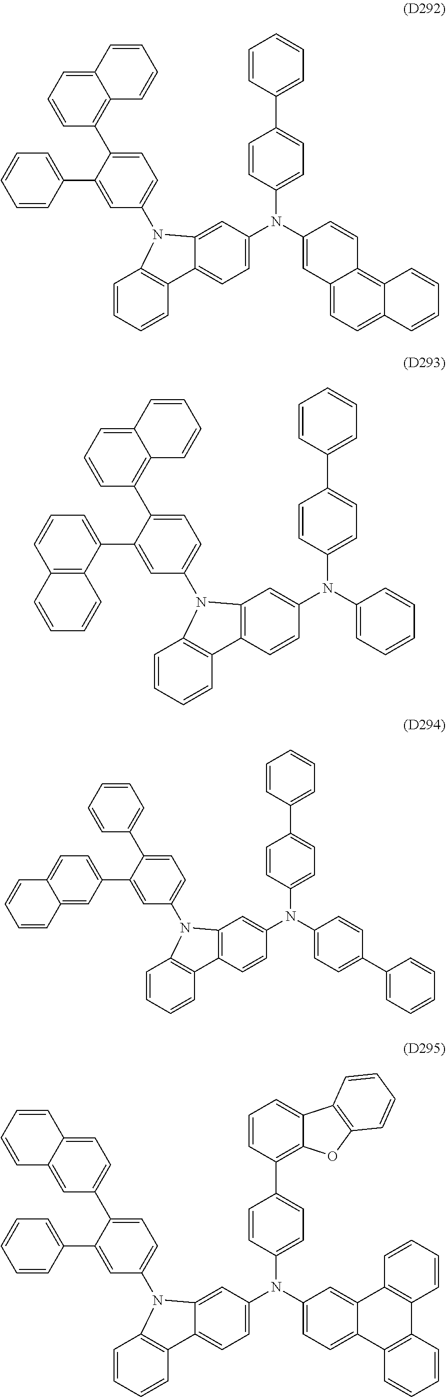 [SMILES: c1ccc(-c2ccc(N(c3ccc4c(ccc5ccccc54)c3)c3ccc4c5ccccc5n(-c5ccc(-c6cccc7ccccc67)c(-c6ccccc6)c5)c4c3)cc2)cc1 ; c1ccc(-c2ccc(N(c3ccccc3)c3ccc4c5ccccc5n(-c5ccc(-c6cccc7ccccc67)c(-c6cccc7ccccc67)c5)c4c3)cc2)cc1 ; c1ccc(-c2ccc(N(c3ccc(-c4ccccc4)cc3)c3ccc4c5ccccc5n(-c5ccc(-c6ccccc6)c(-c6ccc7ccccc7c6)c5)c4c3)cc2)cc1 ; c1ccc(-c2cc(-n3c4ccccc4c4ccc(N(c5ccc(-c6cccc7c6oc6ccccc67)cc5)c5ccc6c7ccccc7c7ccccc7c6c5)cc43)ccc2-c2ccc3ccccc3c2)cc1]

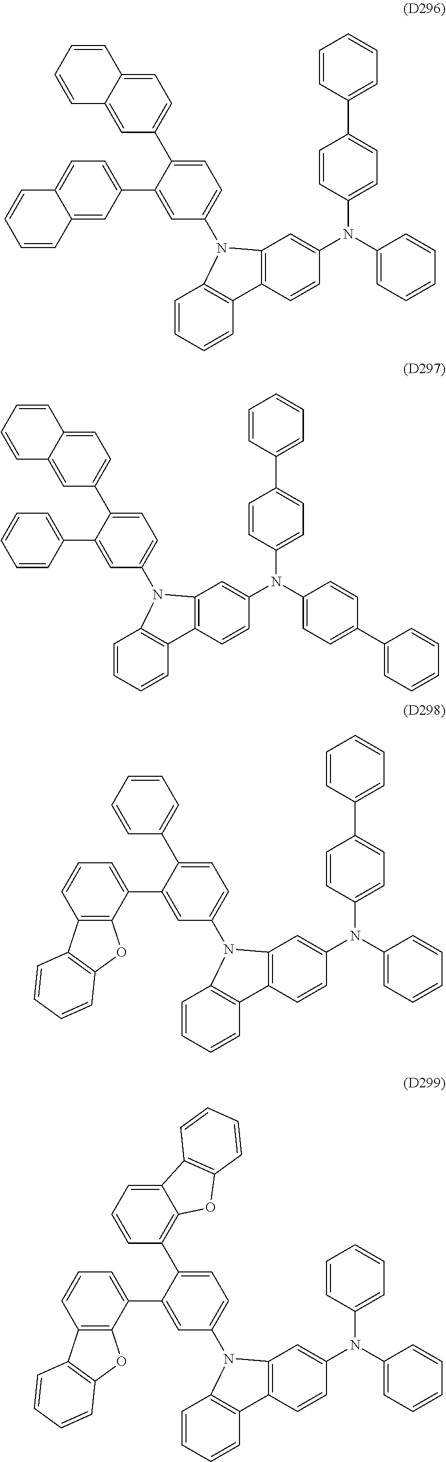 [SMILES: c1ccc(-c2ccc(N(c3ccccc3)c3ccc4c5ccccc5n(-c5ccc(-c6ccc7ccccc7c6)c(-c6ccc7ccccc7c6)c5)c4c3)cc2)cc1 ; c1ccc(-c2ccc(N(c3ccc(-c4ccccc4)cc3)c3ccc4c5ccccc5n(-c5ccc(-c6ccc7ccccc7c6)c(-c6ccccc6)c5)c4c3)cc2)cc1 ; c1ccc(-c2ccc(N(c3ccccc3)c3ccc4c5ccccc5n(-c5ccc(-c6ccccc6)c(-c6cccc7c6oc6ccccc67)c5)c4c3)cc2)cc1 ; c1ccc(N(c2ccccc2)c2ccc3c4ccccc4n(-c4ccc(-c5cccc6c5oc5ccccc56)c(-c5cccc6c5oc5ccccc56)c4)c3c2)cc1]

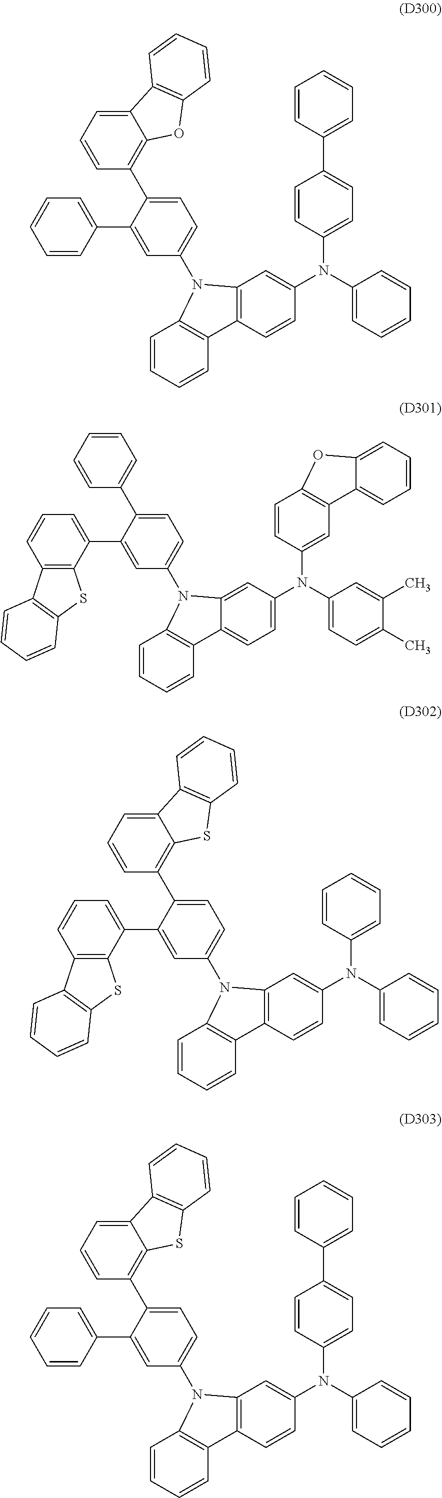 [SMILES: c1ccc(-c2ccc(N(c3ccccc3)c3ccc4c5ccccc5n(-c5ccc(-c6cccc7c6oc6ccccc67)c(-c6ccccc6)c5)c4c3)cc2)cc1 ; Cc1ccc(N(c2ccc3oc4ccccc4c3c2)c2ccc3c4ccccc4n(-c4ccc(-c5ccccc5)c(-c5cccc6c5sc5ccccc56)c4)c3c2)cc1C ; c1ccc(N(c2ccccc2)c2ccc3c4ccccc4n(-c4ccc(-c5cccc6c5sc5ccccc56)c(-c5cccc6c5sc5ccccc56)c4)c3c2)cc1 ; c1ccc(-c2ccc(N(c3ccccc3)c3ccc4c5ccccc5n(-c5ccc(-c6cccc7c6sc6ccccc67)c(-c6ccccc6)c5)c4c3)cc2)cc1]

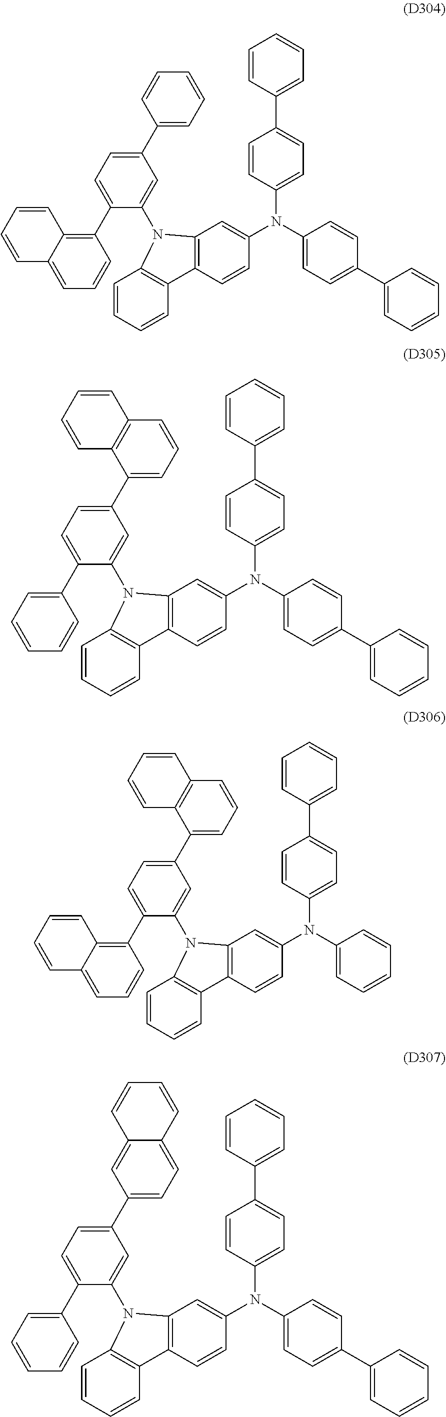

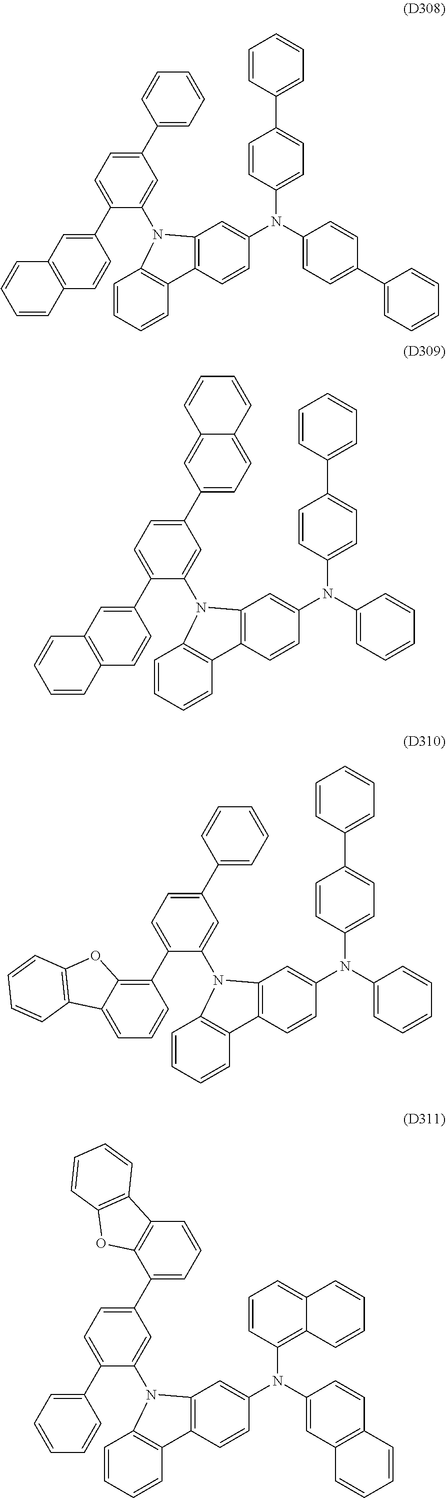 [SMILES: c1ccc(-c2ccc(N(c3ccc(-c4ccccc4)cc3)c3ccc4c5ccccc5n(-c5cc(-c6ccccc6)ccc5-c5ccc6ccccc6c5)c4c3)cc2)cc1 ; c1ccc(-c2ccc(N(c3ccccc3)c3ccc4c5ccccc5n(-c5cc(-c6ccc7ccccc7c6)ccc5-c5ccc6ccccc6c5)c4c3)cc2)cc1 ; c1ccc(-c2ccc(N(c3ccccc3)c3ccc4c5ccccc5n(-c5cc(-c6ccccc6)ccc5-c5cccc6c5oc5ccccc56)c4c3)cc2)cc1 ; c1ccc(-c2ccc(-c3cccc4c3oc3ccccc34)cc2-n2c3ccccc3c3ccc(N(c4ccc5ccccc5c4)c4cccc5ccccc45)cc32)cc1]

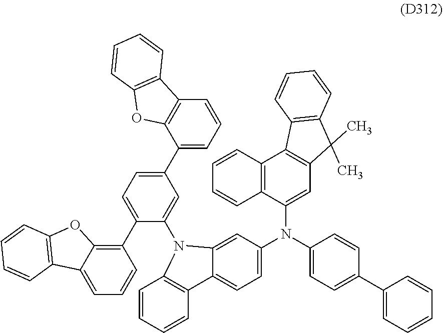 [SMILES: CC1(C)c2ccccc2-c2c1cc(N(c1ccc(-c3ccccc3)cc1)c1ccc3c4ccccc4n(-c4cc(-c5cccc6c5oc5ccccc56)ccc4-c4cccc5c4oc4ccccc45)c3c1)c1ccccc21]

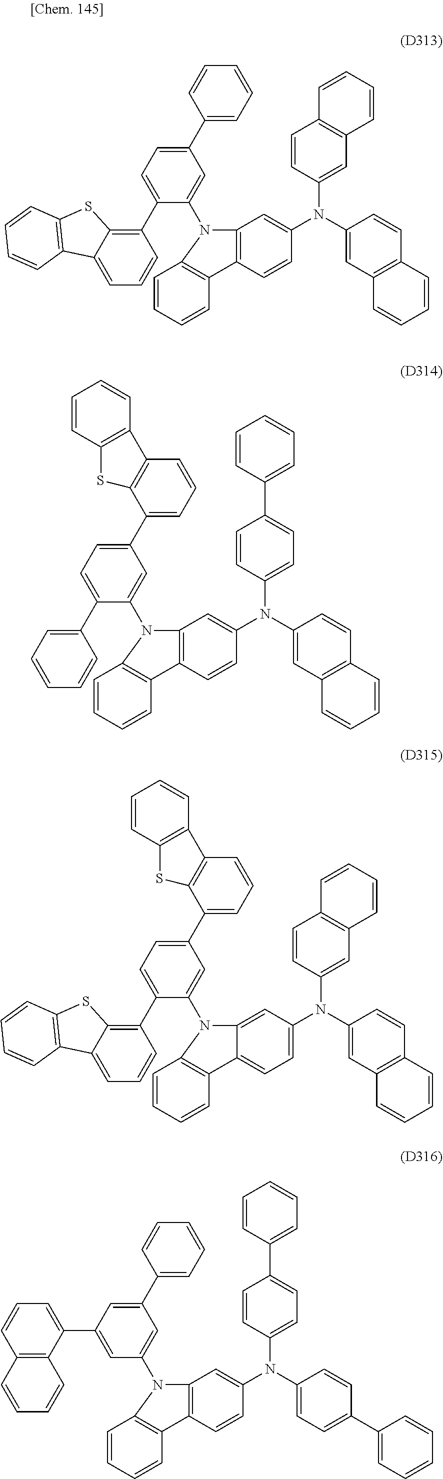 [SMILES: c1ccc(-c2ccc(-c3cccc4c3sc3ccccc34)c(-n3c4ccccc4c4ccc(N(c5ccc6ccccc6c5)c5ccc6ccccc6c5)cc43)c2)cc1 ; c1ccc(-c2ccc(N(c3ccc4ccccc4c3)c3ccc4c5ccccc5n(-c5cc(-c6cccc7c6sc6ccccc67)ccc5-c5ccccc5)c4c3)cc2)cc1 ; c1ccc2cc(N(c3ccc4ccccc4c3)c3ccc4c5ccccc5n(-c5cc(-c6cccc7c6sc6ccccc67)ccc5-c5cccc6c5sc5ccccc56)c4c3)ccc2c1 ; c1ccc(-c2ccc(N(c3ccc(-c4ccccc4)cc3)c3ccc4c5ccccc5n(-c5cc(-c6ccccc6)cc(-c6cccc7ccccc67)c5)c4c3)cc2)cc1]

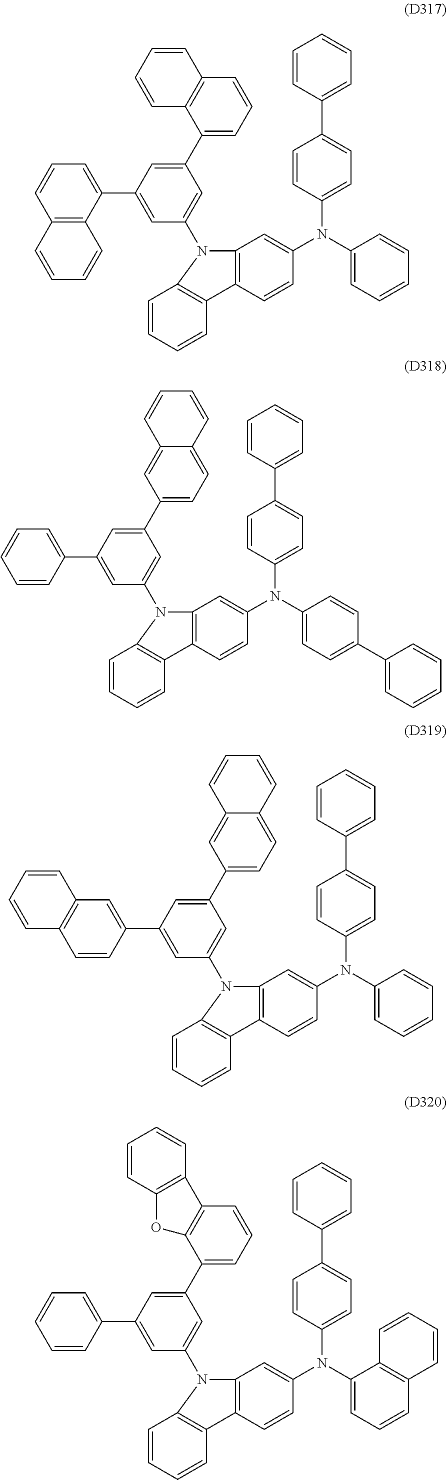

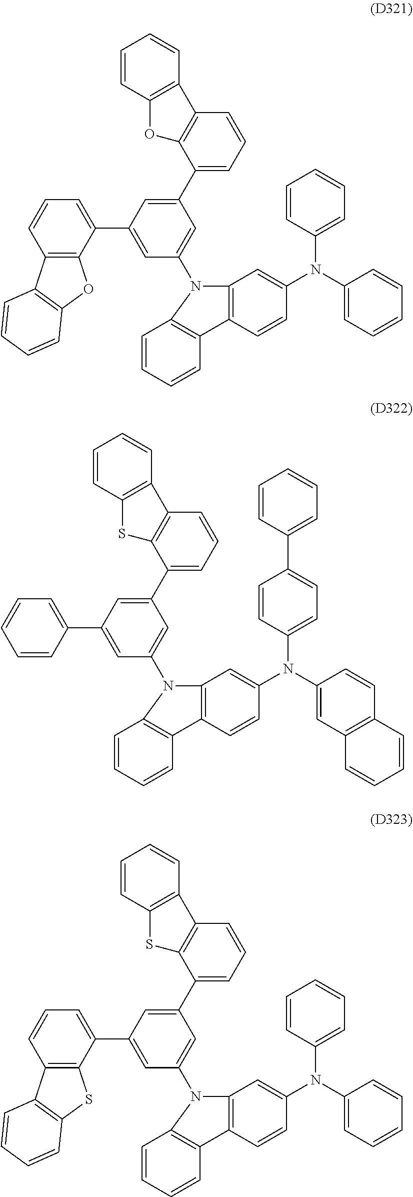 [SMILES: c1ccc(N(c2ccccc2)c2ccc3c4ccccc4n(-c4cc(-c5cccc6c5oc5ccccc56)cc(-c5cccc6c5oc5ccccc56)c4)c3c2)cc1 ; c1ccc(-c2ccc(N(c3ccc4ccccc4c3)c3ccc4c5ccccc5n(-c5cc(-c6ccccc6)cc(-c6cccc7c6sc6ccccc67)c5)c4c3)cc2)cc1 ; c1ccc(N(c2ccccc2)c2ccc3c4ccccc4n(-c4cc(-c5cccc6c5sc5ccccc56)cc(-c5cccc6c5sc5ccccc56)c4)c3c2)cc1]

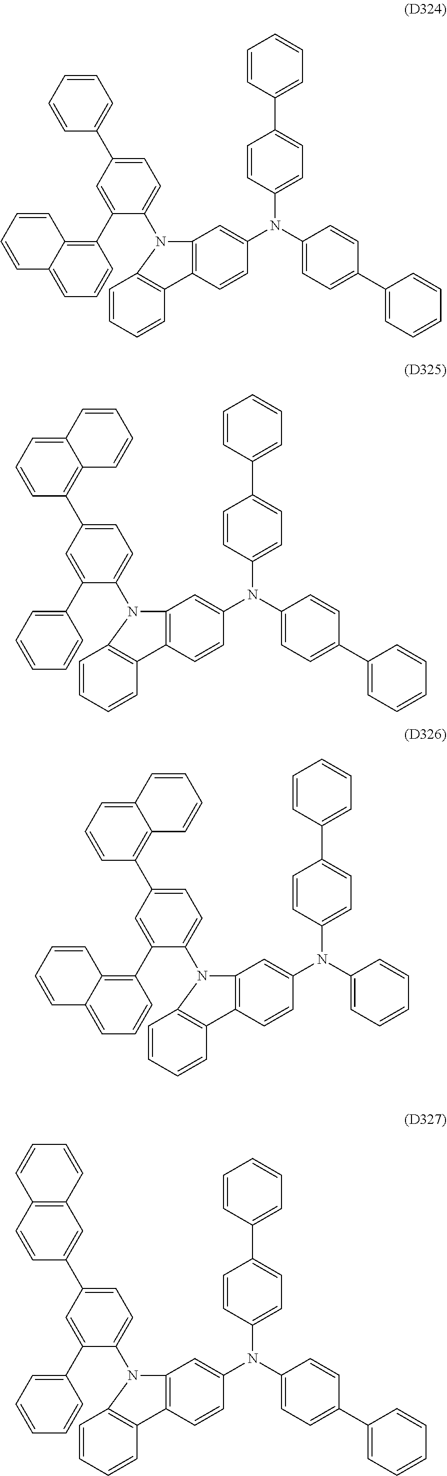 [SMILES: c1ccc(-c2ccc(N(c3ccc(-c4ccccc4)cc3)c3ccc4c5ccccc5n(-c5ccc(-c6ccccc6)cc5-c5cccc6ccccc56)c4c3)cc2)cc1 ; c1ccc(-c2ccc(N(c3ccc(-c4ccccc4)cc3)c3ccc4c5ccccc5n(-c5ccc(-c6cccc7ccccc67)cc5-c5ccccc5)c4c3)cc2)cc1 ; c1ccc(-c2ccc(N(c3ccccc3)c3ccc4c5ccccc5n(-c5ccc(-c6cccc7ccccc67)cc5-c5cccc6ccccc56)c4c3)cc2)cc1 ; c1ccc(-c2ccc(N(c3ccc(-c4ccccc4)cc3)c3ccc4c5ccccc5n(-c5ccc(-c6ccc7ccccc7c6)cc5-c5ccccc5)c4c3)cc2)cc1]

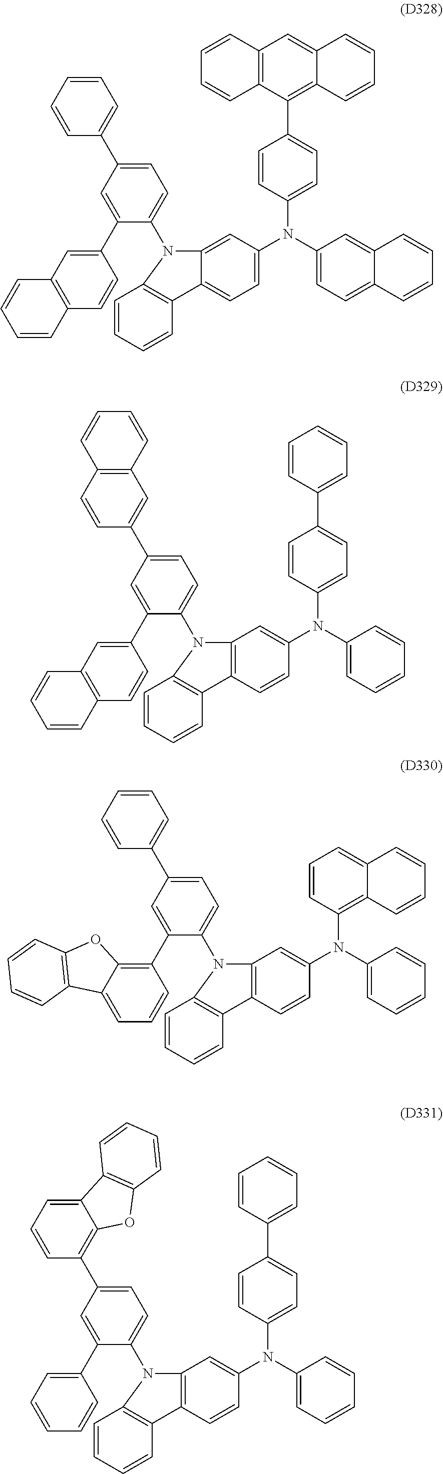 [SMILES: c1ccc(-c2ccc(-n3c4ccccc4c4ccc(N(c5ccc(-c6c7ccccc7cc7ccccc67)cc5)c5ccc6ccccc6c5)cc43)c(-c3ccc4ccccc4c3)c2)cc1 ; c1ccc(-c2ccc(N(c3ccccc3)c3ccc4c5ccccc5n(-c5ccc(-c6ccc7ccccc7c6)cc5-c5ccc6ccccc6c5)c4c3)cc2)cc1 ; c1ccc(-c2ccc(-n3c4ccccc4c4ccc(N(c5ccccc5)c5cccc6ccccc56)cc43)c(-c3cccc4c3oc3ccccc34)c2)cc1 ; c1ccc(-c2ccc(N(c3ccccc3)c3ccc4c5ccccc5n(-c5ccc(-c6cccc7c6oc6ccccc67)cc5-c5ccccc5)c4c3)cc2)cc1]

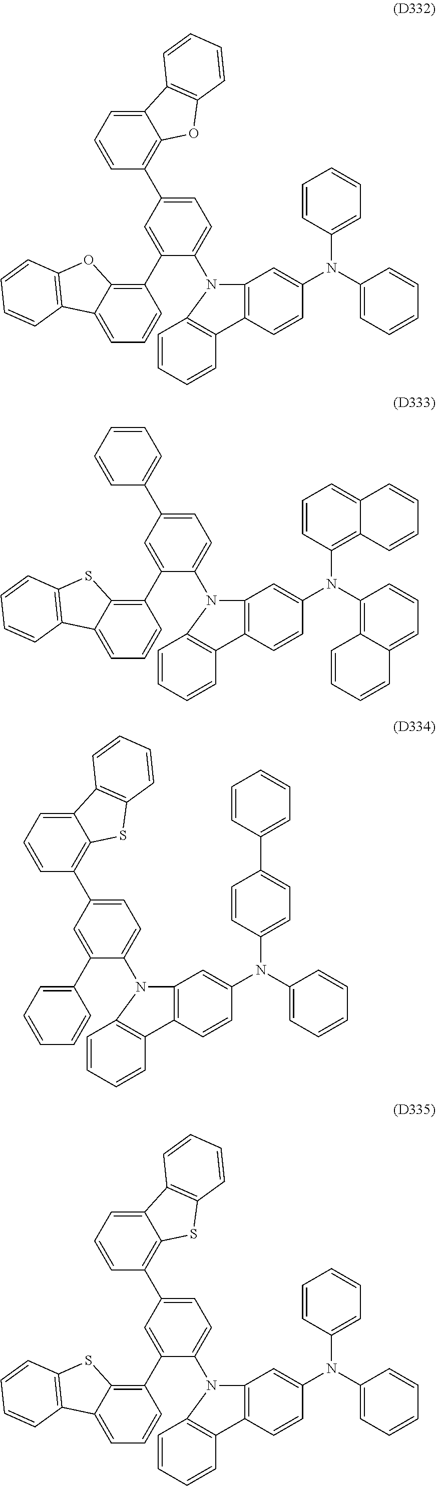 [SMILES: c1ccc(N(c2ccccc2)c2ccc3c4ccccc4n(-c4ccc(-c5cccc6c5oc5ccccc56)cc4-c4cccc5c4oc4ccccc45)c3c2)cc1 ; c1ccc(-c2ccc(-n3c4ccccc4c4ccc(N(c5cccc6ccccc56)c5cccc6ccccc56)cc43)c(-c3cccc4c3sc3ccccc34)c2)cc1 ; c1ccc(-c2ccc(N(c3ccccc3)c3ccc4c5ccccc5n(-c5ccc(-c6cccc7c6sc6ccccc67)cc5-c5ccccc5)c4c3)cc2)cc1 ; c1ccc(N(c2ccccc2)c2ccc3c4ccccc4n(-c4ccc(-c5cccc6c5sc5ccccc56)cc4-c4cccc5c4sc4ccccc45)c3c2)cc1]

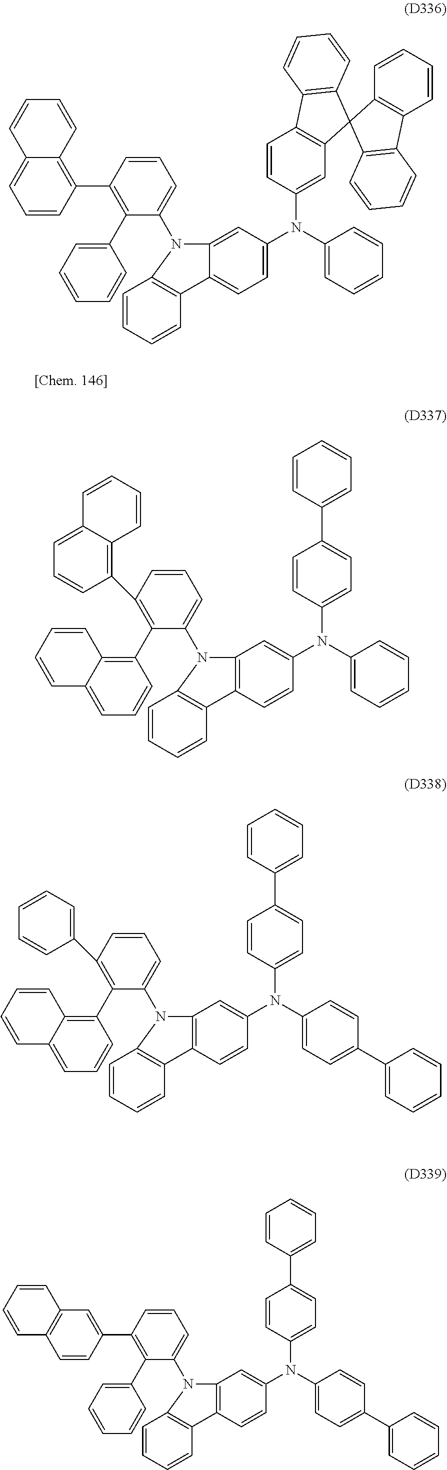 [SMILES: c1ccc(-c2c(-c3cccc4ccccc34)cccc2-n2c3ccccc3c3ccc(N(c4ccccc4)c4ccc5c(c4)C4(c6ccccc6-c6ccccc64)c4ccccc4-5)cc32)cc1 ; c1ccc(-c2ccc(N(c3ccccc3)c3ccc4c5ccccc5n(-c5cccc(-c6cccc7ccccc67)c5-c5cccc6ccccc56)c4c3)cc2)cc1 ; c1ccc(-c2ccc(N(c3ccc(-c4ccccc4)cc3)c3ccc4c5ccccc5n(-c5cccc(-c6ccccc6)c5-c5cccc6ccccc56)c4c3)cc2)cc1 ; c1ccc(-c2ccc(N(c3ccc(-c4ccccc4)cc3)c3ccc4c5ccccc5n(-c5cccc(-c6ccc7ccccc7c6)c5-c5ccccc5)c4c3)cc2)cc1]

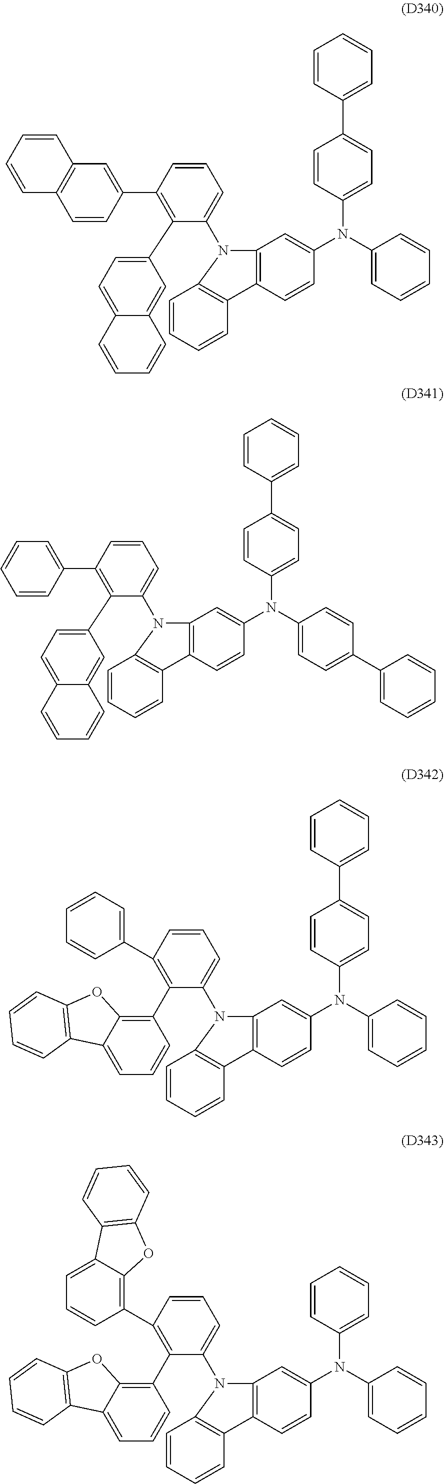 [SMILES: c1ccc(-c2ccc(N(c3ccccc3)c3ccc4c5ccccc5n(-c5cccc(-c6ccc7ccccc7c6)c5-c5ccc6ccccc6c5)c4c3)cc2)cc1 ; c1ccc(-c2ccc(N(c3ccc(-c4ccccc4)cc3)c3ccc4c5ccccc5n(-c5cccc(-c6ccccc6)c5-c5ccc6ccccc6c5)c4c3)cc2)cc1 ; c1ccc(-c2ccc(N(c3ccccc3)c3ccc4c5ccccc5n(-c5cccc(-c6ccccc6)c5-c5cccc6c5oc5ccccc56)c4c3)cc2)cc1 ; c1ccc(N(c2ccccc2)c2ccc3c4ccccc4n(-c4cccc(-c5cccc6c5oc5ccccc56)c4-c4cccc5c4oc4ccccc45)c3c2)cc1]

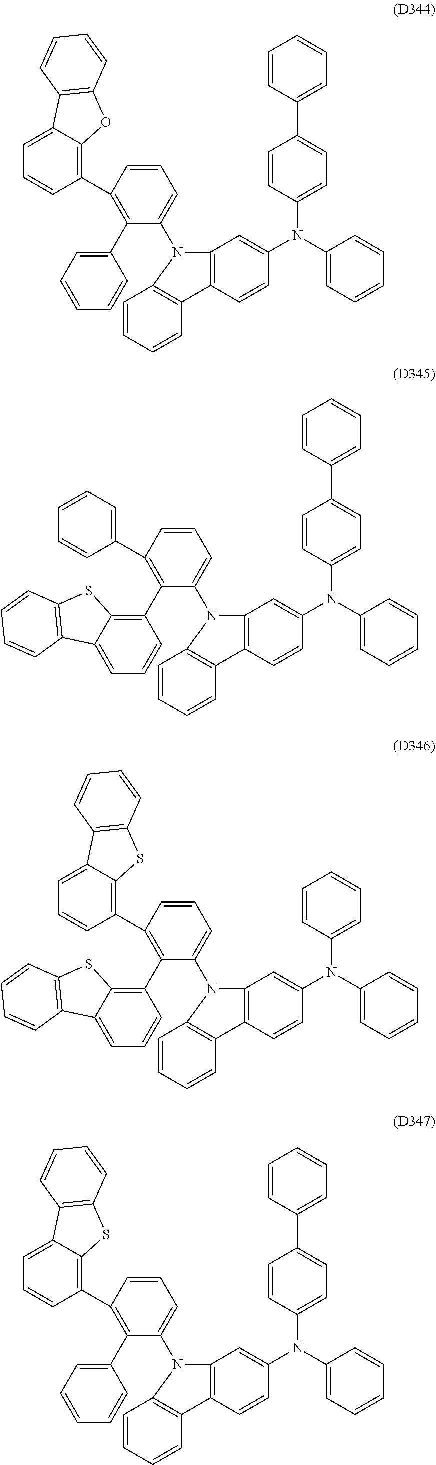 [SMILES: c1ccc(-c2ccc(N(c3ccccc3)c3ccc4c5ccccc5n(-c5cccc(-c6cccc7c6oc6ccccc67)c5-c5ccccc5)c4c3)cc2)cc1 ; c1ccc(-c2ccc(N(c3ccccc3)c3ccc4c5ccccc5n(-c5cccc(-c6ccccc6)c5-c5cccc6c5sc5ccccc56)c4c3)cc2)cc1 ; c1ccc(N(c2ccccc2)c2ccc3c4ccccc4n(-c4cccc(-c5cccc6c5sc5ccccc56)c4-c4cccc5c4sc4ccccc45)c3c2)cc1 ; c1ccc(-c2ccc(N(c3ccccc3)c3ccc4c5ccccc5n(-c5cccc(-c6cccc7c6sc6ccccc67)c5-c5ccccc5)c4c3)cc2)cc1]

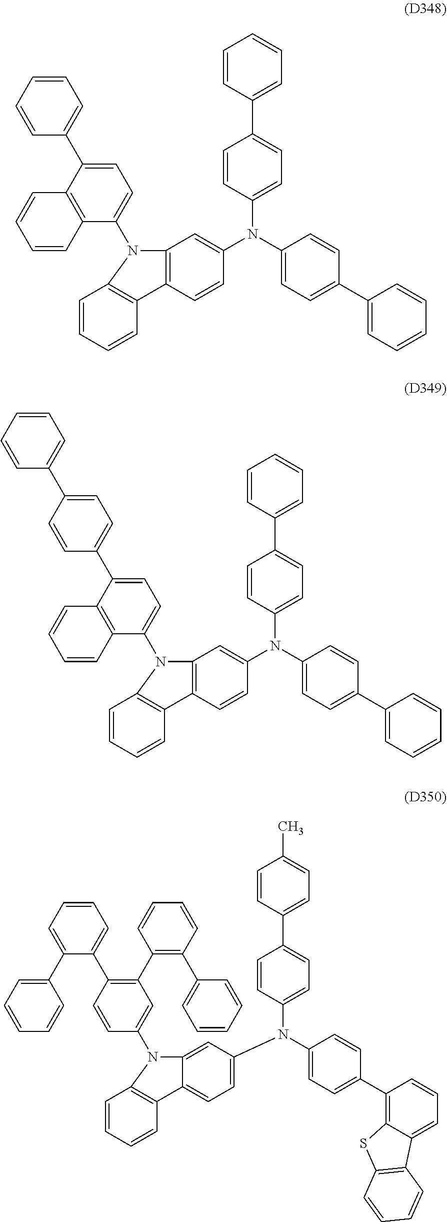 [SMILES: c1ccc(-c2ccc(N(c3ccc(-c4ccccc4)cc3)c3ccc4c5ccccc5n(-c5ccc(-c6ccccc6)c6ccccc56)c4c3)cc2)cc1 ; c1ccc(-c2ccc(-c3ccc(-n4c5ccccc5c5ccc(N(c6ccc(-c7ccccc7)cc6)c6ccc(-c7ccccc7)cc6)cc54)c4ccccc34)cc2)cc1 ; Cc1ccc(-c2ccc(N(c3ccc(-c4cccc5c4sc4ccccc45)cc3)c3ccc4c5ccccc5n(-c5ccc(-c6ccccc6-c6ccccc6)c(-c6ccccc6-c6ccccc6)c5)c4c3)cc2)cc1]

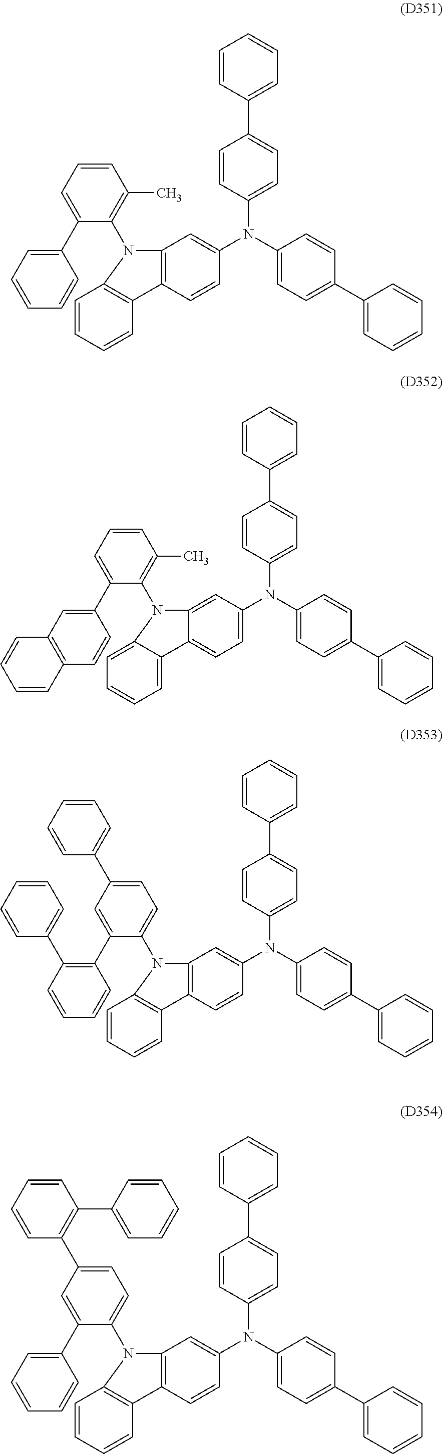 [SMILES: Cc1cccc(-c2ccccc2)c1-n1c2ccccc2c2ccc(N(c3ccc(-c4ccccc4)cc3)c3ccc(-c4ccccc4)cc3)cc21 ; Cc1cccc(-c2ccc3ccccc3c2)c1-n1c2ccccc2c2ccc(N(c3ccc(-c4ccccc4)cc3)c3ccc(-c4ccccc4)cc3)cc21 ; c1ccc(-c2ccc(N(c3ccc(-c4ccccc4)cc3)c3ccc4c5ccccc5n(-c5ccc(-c6ccccc6)cc5-c5ccccc5-c5ccccc5)c4c3)cc2)cc1 ; c1ccc(-c2ccc(N(c3ccc(-c4ccccc4)cc3)c3ccc4c5ccccc5n(-c5ccc(-c6ccccc6-c6ccccc6)cc5-c5ccccc5)c4c3)cc2)cc1]

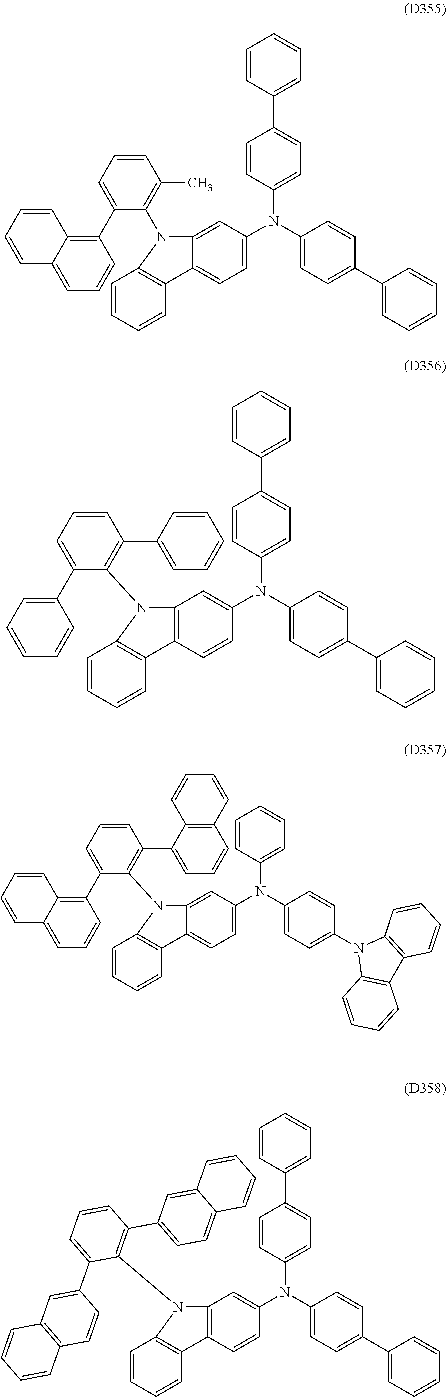 [SMILES: Cc1cccc(-c2cccc3ccccc23)c1-n1c2ccccc2c2ccc(N(c3ccc(-c4ccccc4)cc3)c3ccc(-c4ccccc4)cc3)cc21 ; c1ccc(-c2ccc(N(c3ccc(-c4ccccc4)cc3)c3ccc4c5ccccc5n(-c5c(-c6ccccc6)cccc5-c5ccccc5)c4c3)cc2)cc1 ; c1ccc(N(c2ccc(-n3c4ccccc4c4ccccc43)cc2)c2ccc3c4ccccc4n(-c4c(-c5cccc6ccccc56)cccc4-c4cccc5ccccc45)c3c2)cc1 ; c1ccc(-c2ccc(N(c3ccc(-c4ccccc4)cc3)c3ccc4c5ccccc5n(-c5c(-c6ccc7ccccc7c6)cccc5-c5ccc6ccccc6c5)c4c3)cc2)cc1]

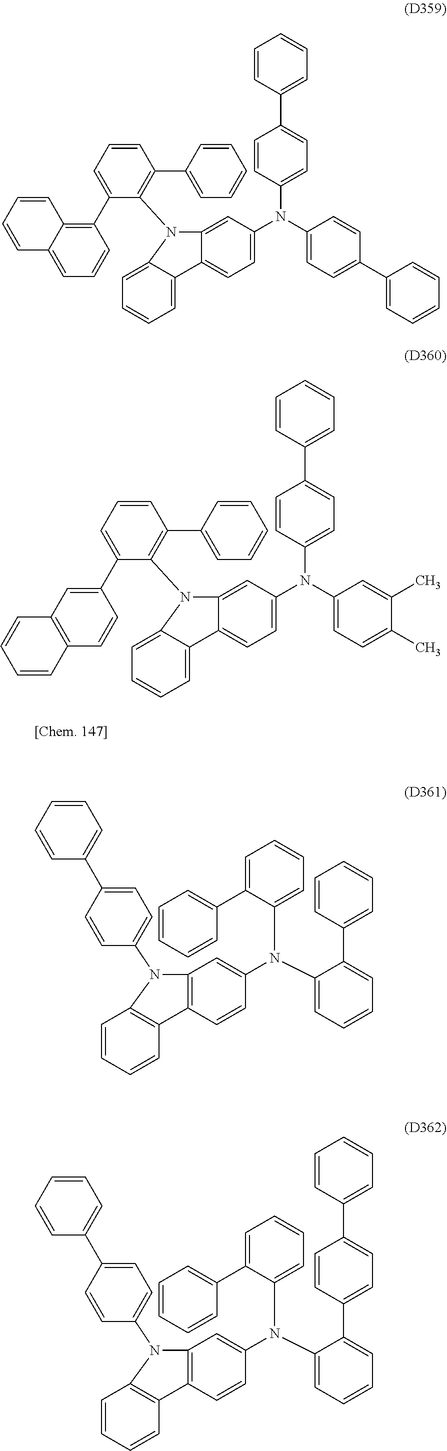 [SMILES: c1ccc(-c2ccc(N(c3ccc(-c4ccccc4)cc3)c3ccc4c5ccccc5n(-c5c(-c6ccccc6)cccc5-c5cccc6ccccc56)c4c3)cc2)cc1 ; Cc1ccc(N(c2ccc(-c3ccccc3)cc2)c2ccc3c4ccccc4n(-c4c(-c5ccccc5)cccc4-c4ccc5ccccc5c4)c3c2)cc1C ; c1ccc(-c2ccc(-n3c4ccccc4c4ccc(N(c5ccccc5-c5ccccc5)c5ccccc5-c5ccccc5)cc43)cc2)cc1 ; c1ccc(-c2ccc(-c3ccccc3N(c3ccc4c5ccccc5n(-c5ccc(-c6ccccc6)cc5)c4c3)c3ccccc3-c3ccccc3)cc2)cc1]

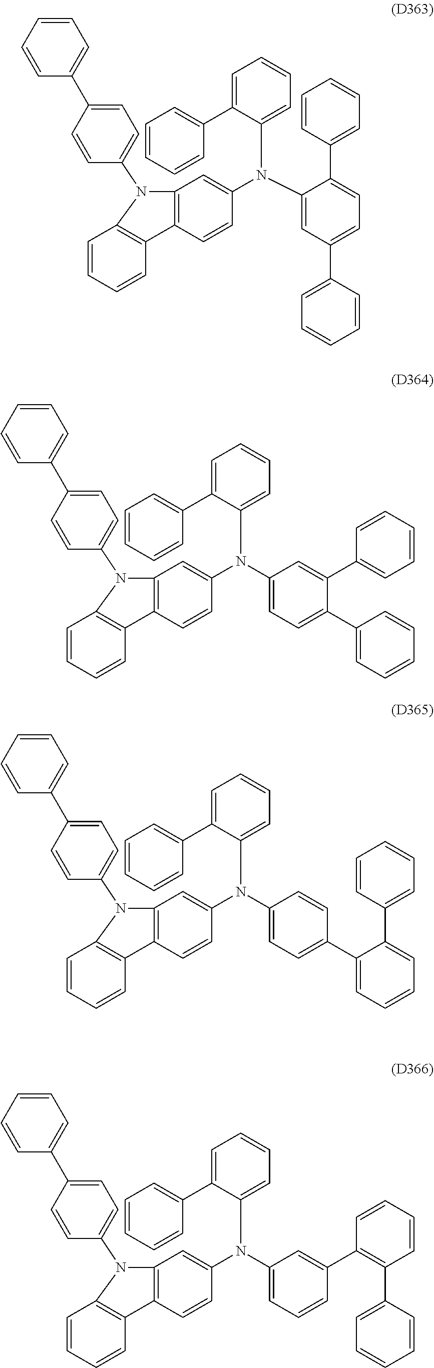 [SMILES: c1ccc(-c2ccc(-n3c4ccccc4c4ccc(N(c5ccccc5-c5ccccc5)c5cc(-c6ccccc6)ccc5-c5ccccc5)cc43)cc2)cc1 ; c1ccc(-c2ccc(-n3c4ccccc4c4ccc(N(c5ccc(-c6ccccc6)c(-c6ccccc6)c5)c5ccccc5-c5ccccc5)cc43)cc2)cc1 ; c1ccc(-c2ccc(-n3c4ccccc4c4ccc(N(c5ccc(-c6ccccc6-c6ccccc6)cc5)c5ccccc5-c5ccccc5)cc43)cc2)cc1 ; c1ccc(-c2ccc(-n3c4ccccc4c4ccc(N(c5cccc(-c6ccccc6-c6ccccc6)c5)c5ccccc5-c5ccccc5)cc43)cc2)cc1]

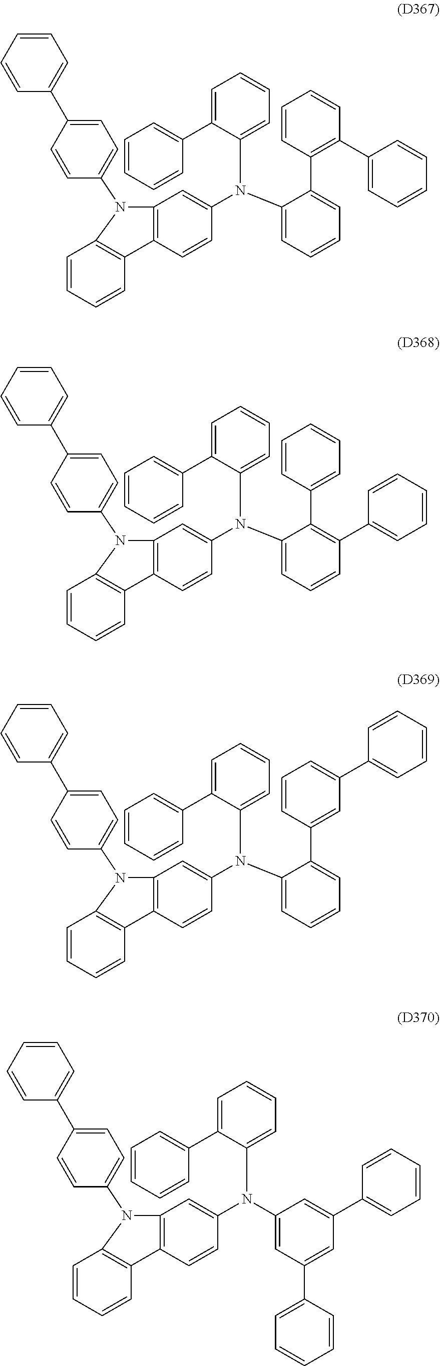 [SMILES: c1ccc(-c2ccc(-n3c4ccccc4c4ccc(N(c5ccccc5-c5ccccc5)c5ccccc5-c5ccccc5-c5ccccc5)cc43)cc2)cc1 ; c1ccc(-c2ccc(-n3c4ccccc4c4ccc(N(c5ccccc5-c5ccccc5)c5cccc(-c6ccccc6)c5-c5ccccc5)cc43)cc2)cc1 ; c1ccc(-c2ccc(-n3c4ccccc4c4ccc(N(c5ccccc5-c5ccccc5)c5ccccc5-c5cccc(-c6ccccc6)c5)cc43)cc2)cc1 ; c1ccc(-c2ccc(-n3c4ccccc4c4ccc(N(c5cc(-c6ccccc6)cc(-c6ccccc6)c5)c5ccccc5-c5ccccc5)cc43)cc2)cc1]

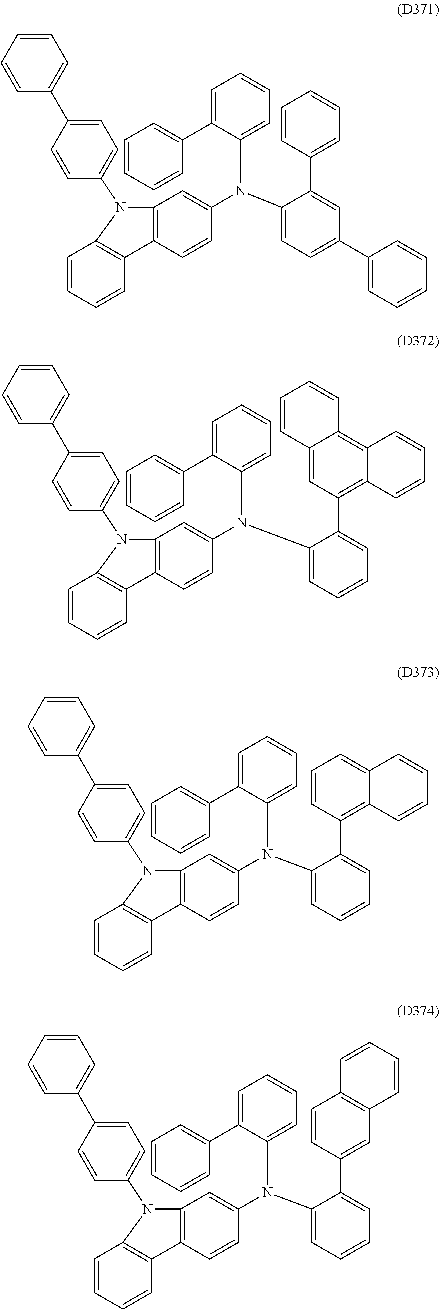 [SMILES: c1ccc(-c2ccc(-n3c4ccccc4c4ccc(N(c5ccccc5-c5ccccc5)c5ccc(-c6ccccc6)cc5-c5ccccc5)cc43)cc2)cc1 ; c1ccc(-c2ccc(-n3c4ccccc4c4ccc(N(c5ccccc5-c5ccccc5)c5ccccc5-c5cc6ccccc6c6ccccc56)cc43)cc2)cc1 ; c1ccc(-c2ccc(-n3c4ccccc4c4ccc(N(c5ccccc5-c5ccccc5)c5ccccc5-c5cccc6ccccc56)cc43)cc2)cc1 ; c1ccc(-c2ccc(-n3c4ccccc4c4ccc(N(c5ccccc5-c5ccccc5)c5ccccc5-c5ccc6ccccc6c5)cc43)cc2)cc1]

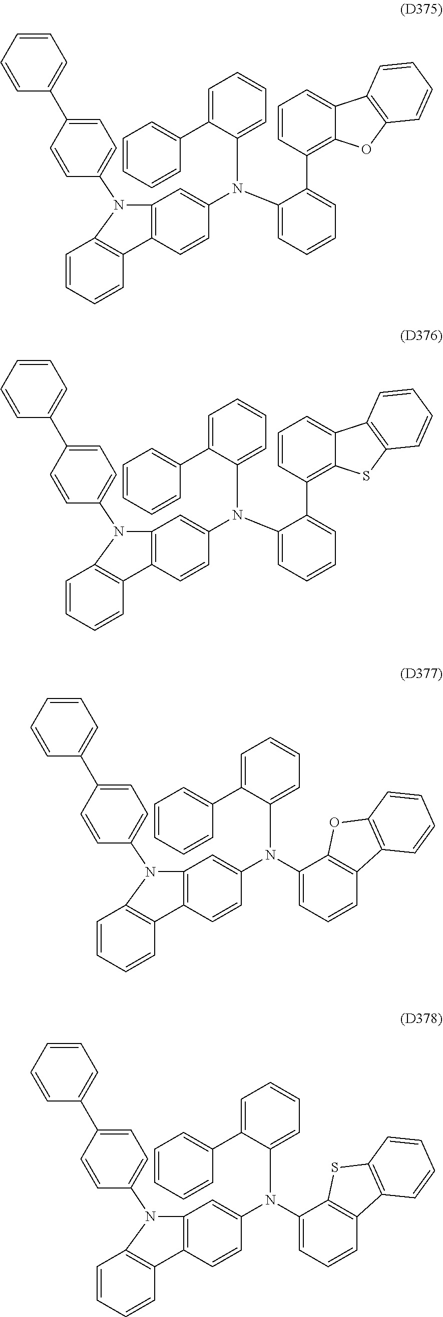 [SMILES: c1ccc(-c2ccc(-n3c4ccccc4c4ccc(N(c5ccccc5-c5ccccc5)c5ccccc5-c5cccc6c5oc5ccccc56)cc43)cc2)cc1 ; c1ccc(-c2ccc(-n3c4ccccc4c4ccc(N(c5ccccc5-c5ccccc5)c5ccccc5-c5cccc6c5sc5ccccc56)cc43)cc2)cc1 ; c1ccc(-c2ccc(-n3c4ccccc4c4ccc(N(c5ccccc5-c5ccccc5)c5cccc6c5oc5ccccc56)cc43)cc2)cc1 ; c1ccc(-c2ccc(-n3c4ccccc4c4ccc(N(c5ccccc5-c5ccccc5)c5cccc6c5sc5ccccc56)cc43)cc2)cc1]

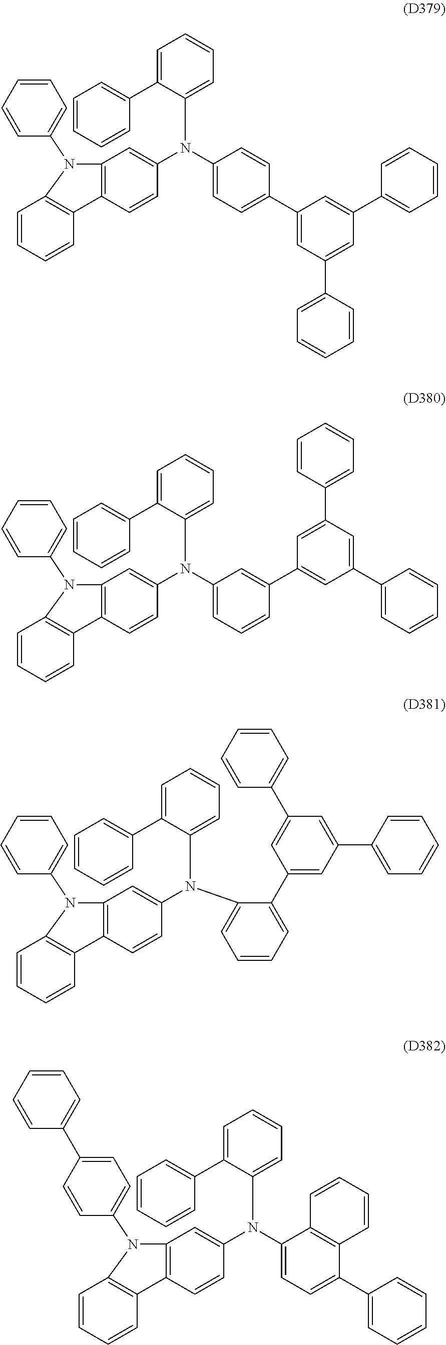 [SMILES: c1ccc(-c2cc(-c3ccccc3)cc(-c3ccc(N(c4ccc5c6ccccc6n(-c6ccccc6)c5c4)c4ccccc4-c4ccccc4)cc3)c2)cc1 ; c1ccc(-c2cc(-c3ccccc3)cc(-c3cccc(N(c4ccc5c6ccccc6n(-c6ccccc6)c5c4)c4ccccc4-c4ccccc4)c3)c2)cc1 ; c1ccc(-c2cc(-c3ccccc3)cc(-c3ccccc3N(c3ccc4c5ccccc5n(-c5ccccc5)c4c3)c3ccccc3-c3ccccc3)c2)cc1 ; c1ccc(-c2ccc(-n3c4ccccc4c4ccc(N(c5ccccc5-c5ccccc5)c5ccc(-c6ccccc6)c6ccccc56)cc43)cc2)cc1]

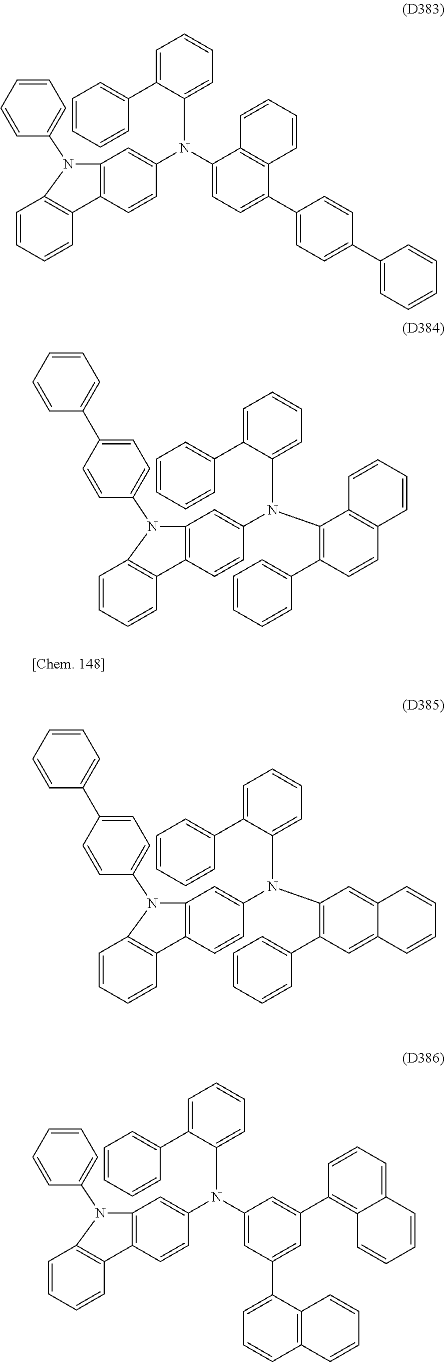 [SMILES: c1ccc(-c2ccc(-c3ccc(N(c4ccc5c6ccccc6n(-c6ccccc6)c5c4)c4ccccc4-c4ccccc4)c4ccccc34)cc2)cc1 ; c1ccc(-c2ccc(-n3c4ccccc4c4ccc(N(c5ccccc5-c5ccccc5)c5c(-c6ccccc6)ccc6ccccc56)cc43)cc2)cc1 ; c1ccc(-c2ccc(-n3c4ccccc4c4ccc(N(c5ccccc5-c5ccccc5)c5cc6ccccc6cc5-c5ccccc5)cc43)cc2)cc1 ; c1ccc(-c2ccccc2N(c2cc(-c3cccc4ccccc34)cc(-c3cccc4ccccc34)c2)c2ccc3c4ccccc4n(-c4ccccc4)c3c2)cc1]

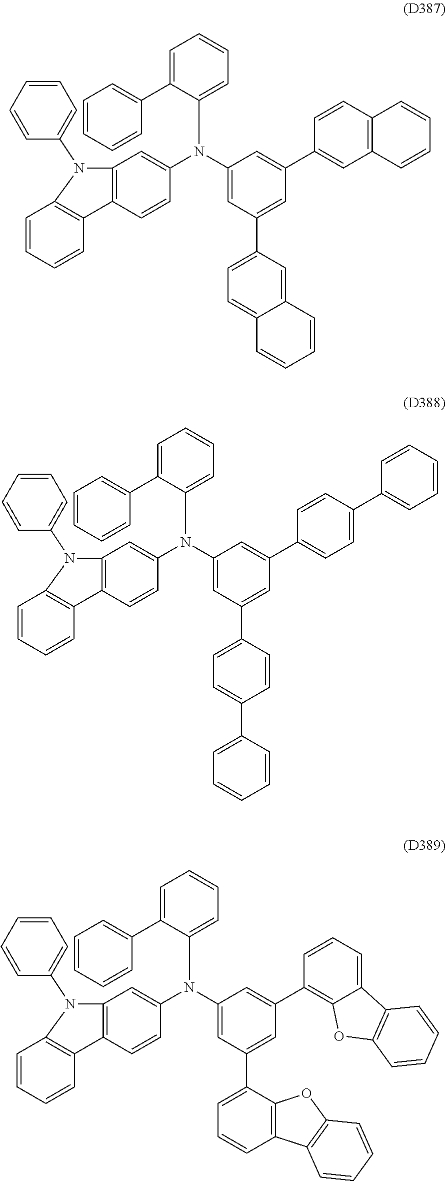 [SMILES: c1ccc(-c2ccccc2N(c2cc(-c3ccc4ccccc4c3)cc(-c3ccc4ccccc4c3)c2)c2ccc3c4ccccc4n(-c4ccccc4)c3c2)cc1 ; c1ccc(-c2ccc(-c3cc(-c4ccc(-c5ccccc5)cc4)cc(N(c4ccc5c6ccccc6n(-c6ccccc6)c5c4)c4ccccc4-c4ccccc4)c3)cc2)cc1 ; c1ccc(-c2ccccc2N(c2cc(-c3cccc4c3oc3ccccc34)cc(-c3cccc4c3oc3ccccc34)c2)c2ccc3c4ccccc4n(-c4ccccc4)c3c2)cc1]

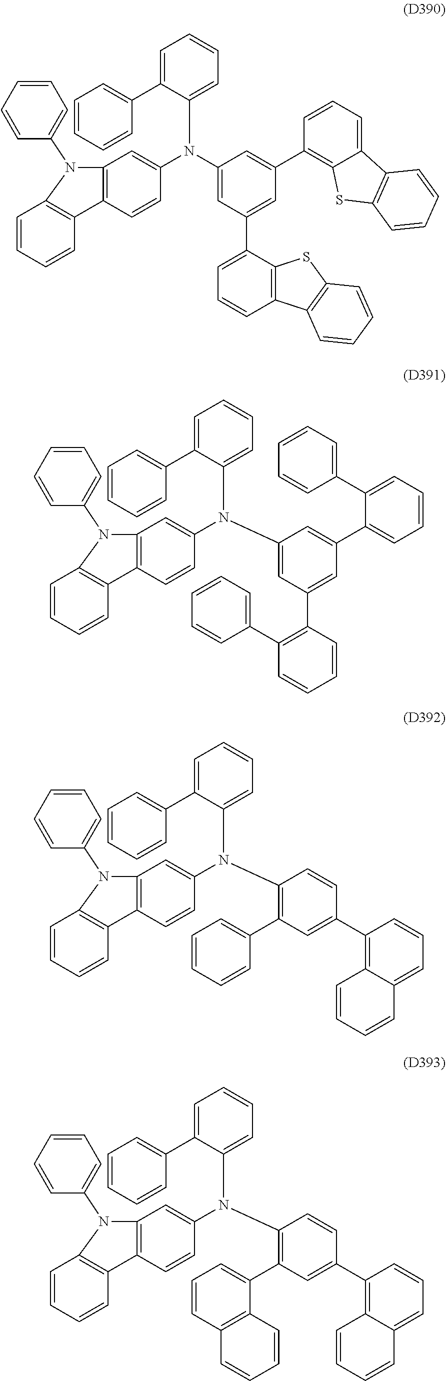 [SMILES: c1ccc(-c2ccccc2N(c2cc(-c3cccc4c3sc3ccccc34)cc(-c3cccc4c3sc3ccccc34)c2)c2ccc3c4ccccc4n(-c4ccccc4)c3c2)cc1 ; c1ccc(-c2ccccc2-c2cc(-c3ccccc3-c3ccccc3)cc(N(c3ccc4c5ccccc5n(-c5ccccc5)c4c3)c3ccccc3-c3ccccc3)c2)cc1 ; c1ccc(-c2ccccc2N(c2ccc3c4ccccc4n(-c4ccccc4)c3c2)c2ccc(-c3cccc4ccccc34)cc2-c2ccccc2)cc1 ; c1ccc(-c2ccccc2N(c2ccc3c4ccccc4n(-c4ccccc4)c3c2)c2ccc(-c3cccc4ccccc34)cc2-c2cccc3ccccc23)cc1]

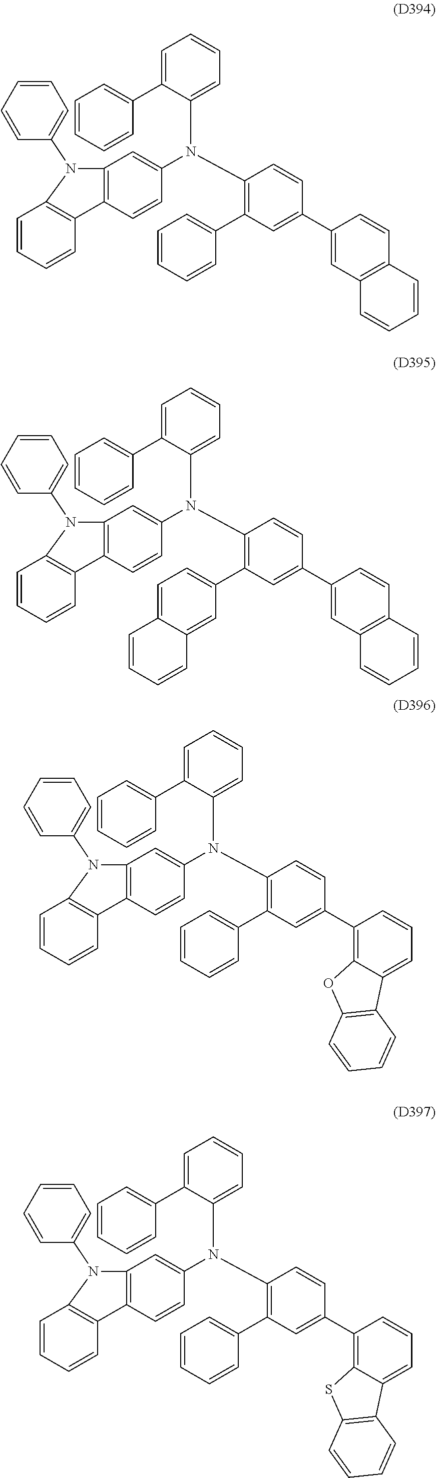 [SMILES: c1ccc(-c2ccccc2N(c2ccc3c4ccccc4n(-c4ccccc4)c3c2)c2ccc(-c3ccc4ccccc4c3)cc2-c2ccccc2)cc1 ; c1ccc(-c2ccccc2N(c2ccc3c4ccccc4n(-c4ccccc4)c3c2)c2ccc(-c3ccc4ccccc4c3)cc2-c2ccc3ccccc3c2)cc1 ; c1ccc(-c2ccccc2N(c2ccc3c4ccccc4n(-c4ccccc4)c3c2)c2ccc(-c3cccc4c3oc3ccccc34)cc2-c2ccccc2)cc1 ; c1ccc(-c2ccccc2N(c2ccc3c4ccccc4n(-c4ccccc4)c3c2)c2ccc(-c3cccc4c3sc3ccccc34)cc2-c2ccccc2)cc1]

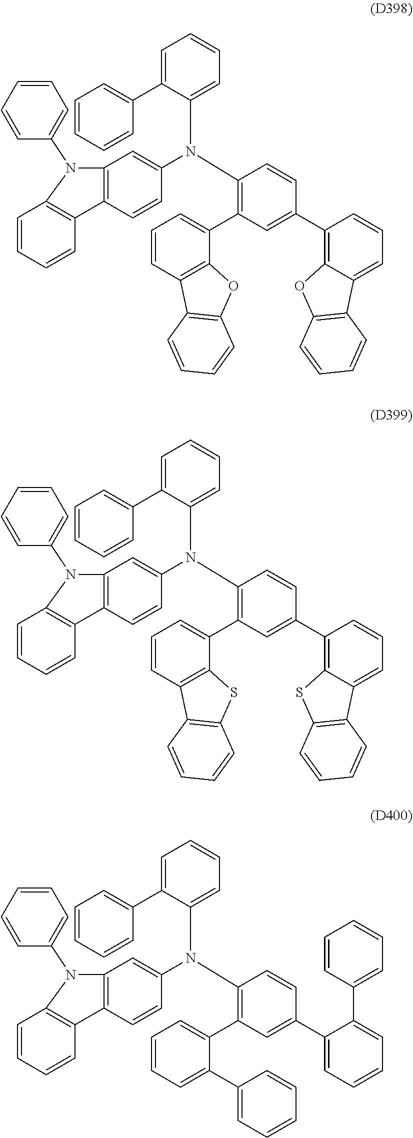 [SMILES: c1ccc(-c2ccccc2N(c2ccc3c4ccccc4n(-c4ccccc4)c3c2)c2ccc(-c3cccc4c3oc3ccccc34)cc2-c2cccc3c2oc2ccccc23)cc1 ; c1ccc(-c2ccccc2N(c2ccc3c4ccccc4n(-c4ccccc4)c3c2)c2ccc(-c3cccc4c3sc3ccccc34)cc2-c2cccc3c2sc2ccccc23)cc1 ; c1ccc(-c2ccccc2-c2ccc(N(c3ccc4c5ccccc5n(-c5ccccc5)c4c3)c3ccccc3-c3ccccc3)c(-c3ccccc3-c3ccccc3)c2)cc1]

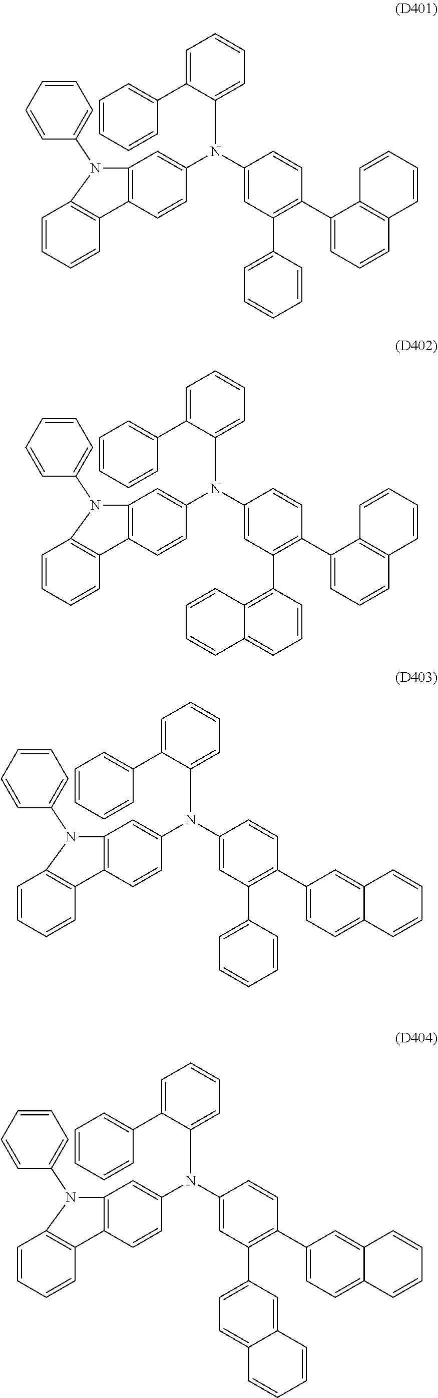 [SMILES: c1ccc(-c2cc(N(c3ccc4c5ccccc5n(-c5ccccc5)c4c3)c3ccccc3-c3ccccc3)ccc2-c2cccc3ccccc23)cc1 ; c1ccc(-c2ccccc2N(c2ccc(-c3cccc4ccccc34)c(-c3cccc4ccccc34)c2)c2ccc3c4ccccc4n(-c4ccccc4)c3c2)cc1 ; c1ccc(-c2cc(N(c3ccc4c5ccccc5n(-c5ccccc5)c4c3)c3ccccc3-c3ccccc3)ccc2-c2ccc3ccccc3c2)cc1 ; c1ccc(-c2ccccc2N(c2ccc(-c3ccc4ccccc4c3)c(-c3ccc4ccccc4c3)c2)c2ccc3c4ccccc4n(-c4ccccc4)c3c2)cc1]

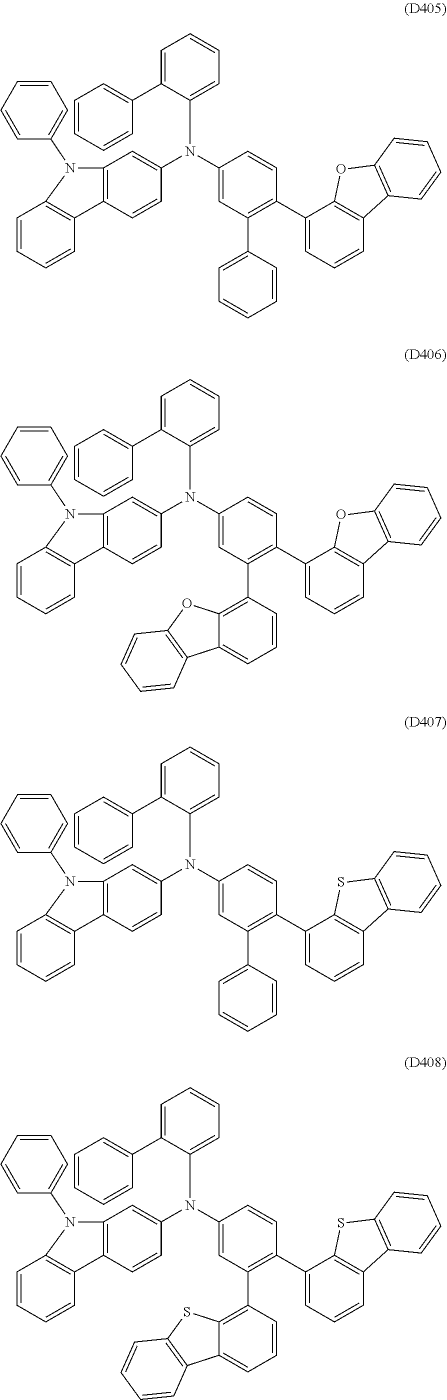 [SMILES: c1ccc(-c2cc(N(c3ccc4c5ccccc5n(-c5ccccc5)c4c3)c3ccccc3-c3ccccc3)ccc2-c2cccc3c2oc2ccccc23)cc1 ; c1ccc(-c2ccccc2N(c2ccc(-c3cccc4c3oc3ccccc34)c(-c3cccc4c3oc3ccccc34)c2)c2ccc3c4ccccc4n(-c4ccccc4)c3c2)cc1 ; c1ccc(-c2cc(N(c3ccc4c5ccccc5n(-c5ccccc5)c4c3)c3ccccc3-c3ccccc3)ccc2-c2cccc3c2sc2ccccc23)cc1 ; c1ccc(-c2ccccc2N(c2ccc(-c3cccc4c3sc3ccccc34)c(-c3cccc4c3sc3ccccc34)c2)c2ccc3c4ccccc4n(-c4ccccc4)c3c2)cc1]

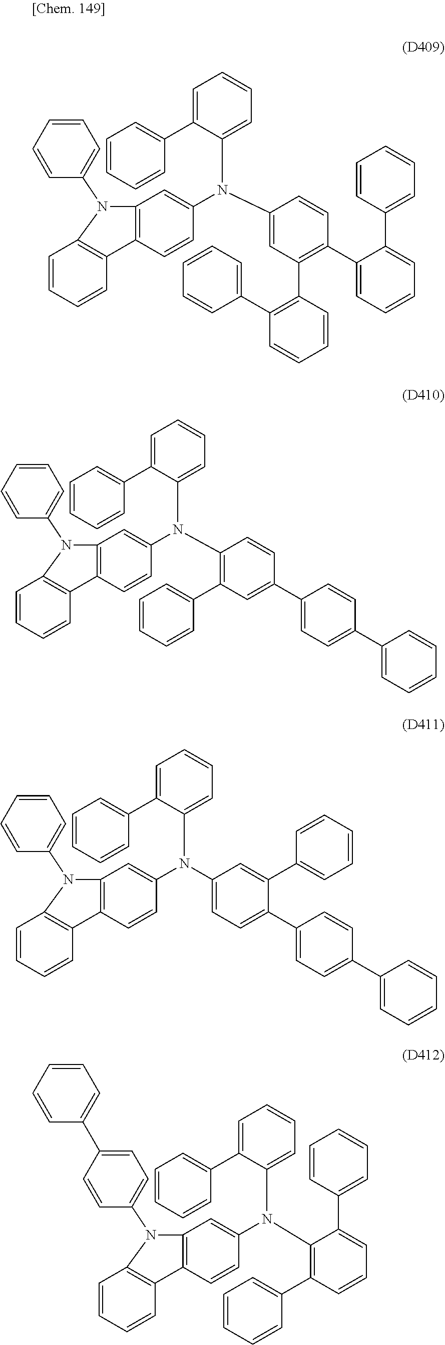 [SMILES: c1ccc(-c2ccccc2-c2ccc(N(c3ccc4c5ccccc5n(-c5ccccc5)c4c3)c3ccccc3-c3ccccc3)cc2-c2ccccc2-c2ccccc2)cc1 ; c1ccc(-c2ccc(-c3ccc(N(c4ccc5c6ccccc6n(-c6ccccc6)c5c4)c4ccccc4-c4ccccc4)c(-c4ccccc4)c3)cc2)cc1 ; c1ccc(-c2ccc(-c3ccc(N(c4ccc5c6ccccc6n(-c6ccccc6)c5c4)c4ccccc4-c4ccccc4)cc3-c3ccccc3)cc2)cc1 ; c1ccc(-c2ccc(-n3c4ccccc4c4ccc(N(c5ccccc5-c5ccccc5)c5c(-c6ccccc6)cccc5-c5ccccc5)cc43)cc2)cc1]

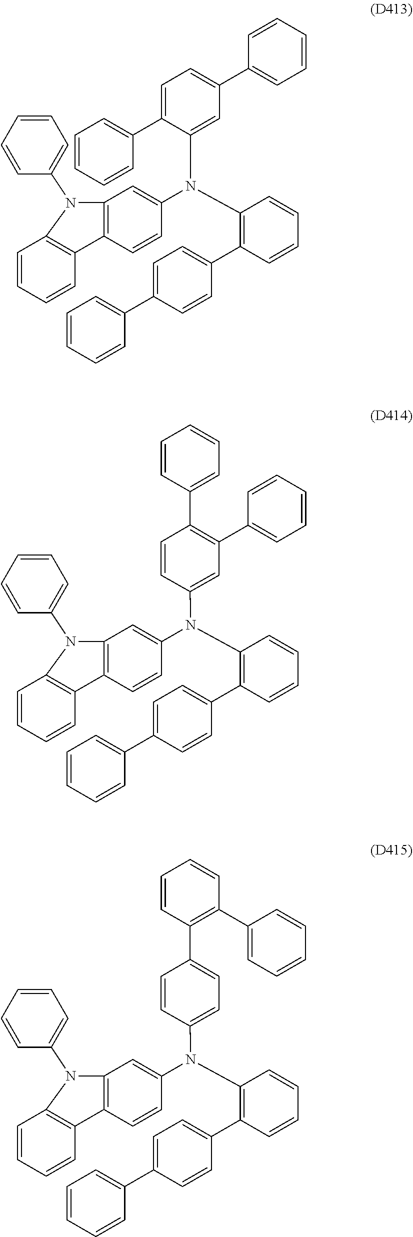 [SMILES: c1ccc(-c2ccc(-c3ccccc3N(c3ccc4c5ccccc5n(-c5ccccc5)c4c3)c3cc(-c4ccccc4)ccc3-c3ccccc3)cc2)cc1 ; c1ccc(-c2ccc(-c3ccccc3N(c3ccc(-c4ccccc4)c(-c4ccccc4)c3)c3ccc4c5ccccc5n(-c5ccccc5)c4c3)cc2)cc1 ; c1ccc(-c2ccc(-c3ccccc3N(c3ccc(-c4ccccc4-c4ccccc4)cc3)c3ccc4c5ccccc5n(-c5ccccc5)c4c3)cc2)cc1]

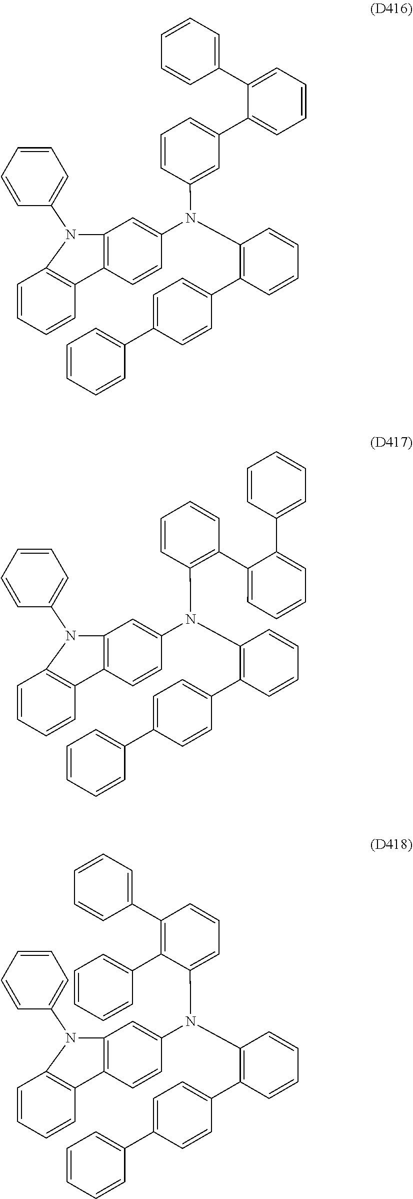 [SMILES: c1ccc(-c2ccc(-c3ccccc3N(c3cccc(-c4ccccc4-c4ccccc4)c3)c3ccc4c5ccccc5n(-c5ccccc5)c4c3)cc2)cc1 ; c1ccc(-c2ccc(-c3ccccc3N(c3ccc4c5ccccc5n(-c5ccccc5)c4c3)c3ccccc3-c3ccccc3-c3ccccc3)cc2)cc1 ; c1ccc(-c2ccc(-c3ccccc3N(c3ccc4c5ccccc5n(-c5ccccc5)c4c3)c3cccc(-c4ccccc4)c3-c3ccccc3)cc2)cc1]

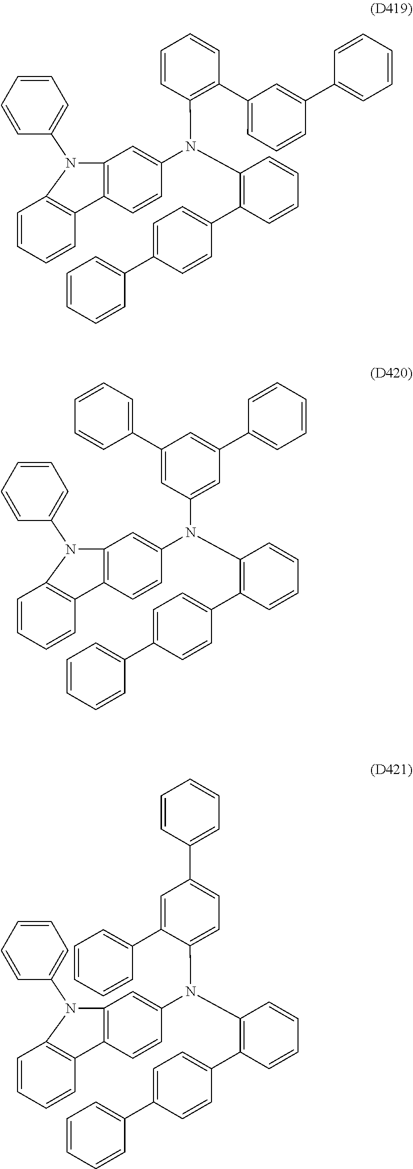 [SMILES: c1ccc(-c2ccc(-c3ccccc3N(c3ccc4c5ccccc5n(-c5ccccc5)c4c3)c3ccccc3-c3cccc(-c4ccccc4)c3)cc2)cc1 ; c1ccc(-c2ccc(-c3ccccc3N(c3cc(-c4ccccc4)cc(-c4ccccc4)c3)c3ccc4c5ccccc5n(-c5ccccc5)c4c3)cc2)cc1 ; c1ccc(-c2ccc(-c3ccccc3N(c3ccc4c5ccccc5n(-c5ccccc5)c4c3)c3ccc(-c4ccccc4)cc3-c3ccccc3)cc2)cc1]

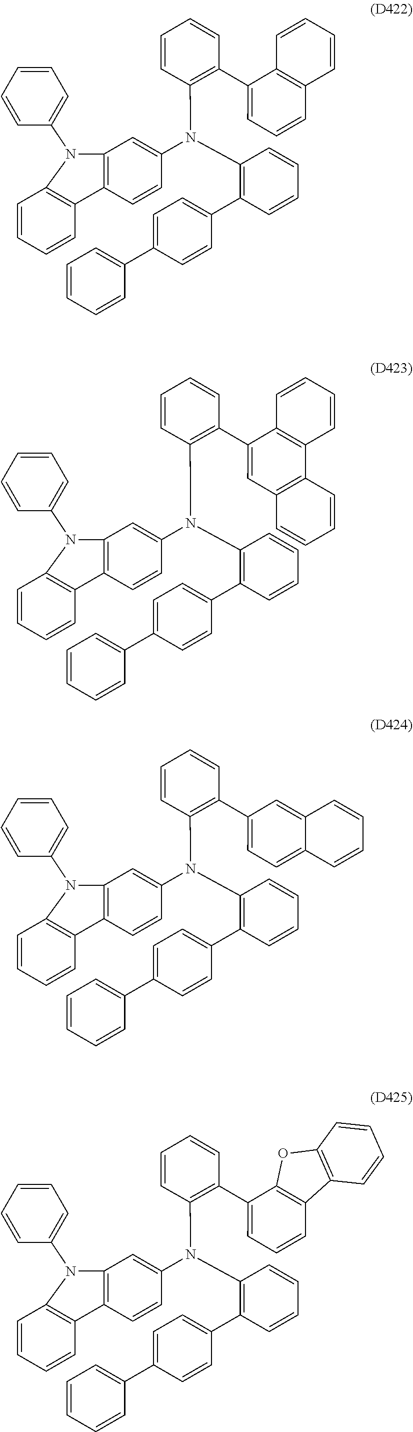 [SMILES: c1ccc(-c2ccc(-c3ccccc3N(c3ccc4c5ccccc5n(-c5ccccc5)c4c3)c3ccccc3-c3cccc4ccccc34)cc2)cc1 ; c1ccc(-c2ccc(-c3ccccc3N(c3ccc4c5ccccc5n(-c5ccccc5)c4c3)c3ccccc3-c3cc4ccccc4c4ccccc34)cc2)cc1 ; c1ccc(-c2ccc(-c3ccccc3N(c3ccc4c5ccccc5n(-c5ccccc5)c4c3)c3ccccc3-c3ccc4ccccc4c3)cc2)cc1 ; c1ccc(-c2ccc(-c3ccccc3N(c3ccc4c5ccccc5n(-c5ccccc5)c4c3)c3ccccc3-c3cccc4c3oc3ccccc34)cc2)cc1]

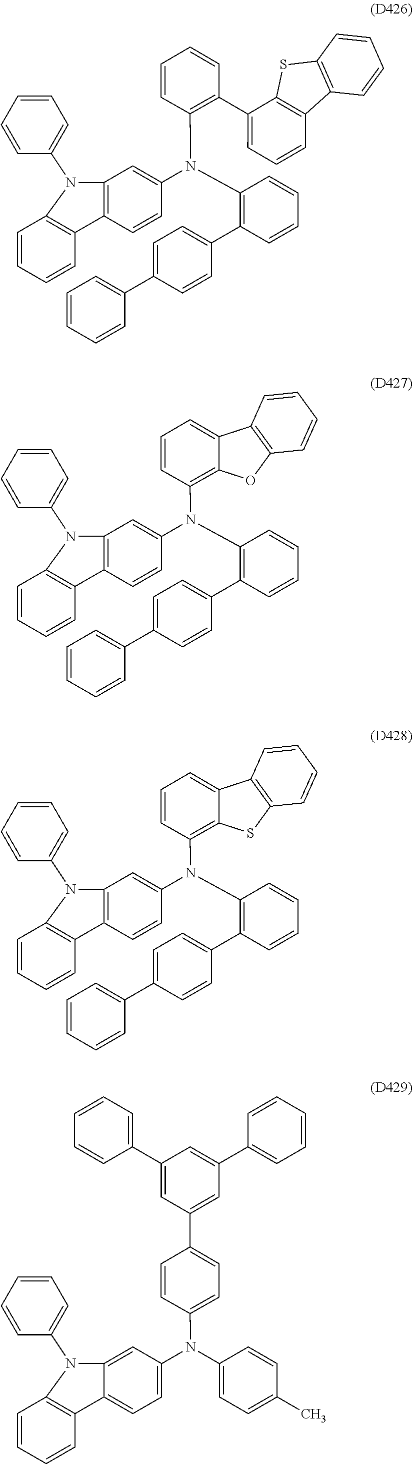 [SMILES: c1ccc(-c2ccc(-c3ccccc3N(c3ccc4c5ccccc5n(-c5ccccc5)c4c3)c3ccccc3-c3cccc4c3sc3ccccc34)cc2)cc1 ; c1ccc(-c2ccc(-c3ccccc3N(c3ccc4c5ccccc5n(-c5ccccc5)c4c3)c3cccc4c3oc3ccccc34)cc2)cc1 ; c1ccc(-c2ccc(-c3ccccc3N(c3ccc4c5ccccc5n(-c5ccccc5)c4c3)c3cccc4c3sc3ccccc34)cc2)cc1 ; Cc1ccc(N(c2ccc(-c3cc(-c4ccccc4)cc(-c4ccccc4)c3)cc2)c2ccc3c4ccccc4n(-c4ccccc4)c3c2)cc1]

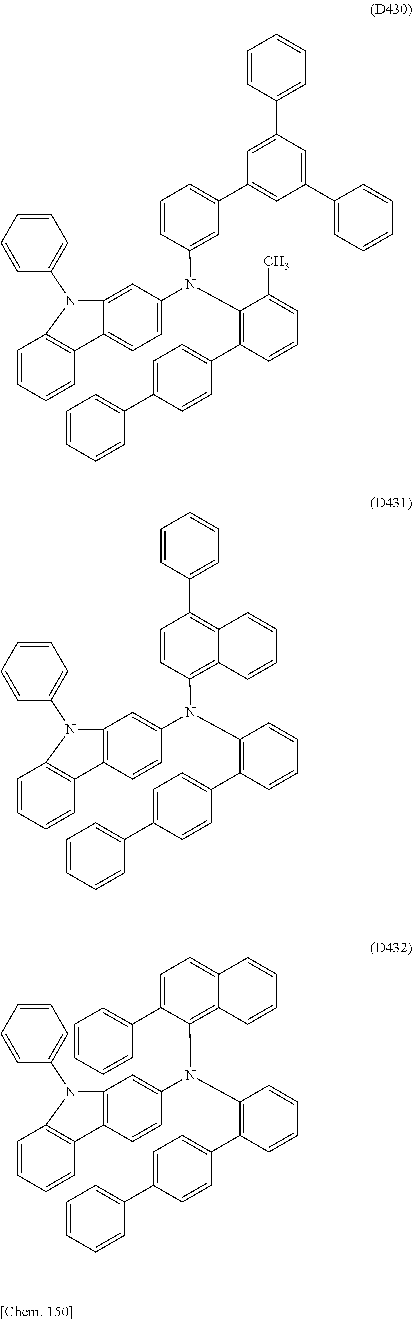 [SMILES: Cc1cccc(-c2ccc(-c3ccccc3)cc2)c1N(c1cccc(-c2cc(-c3ccccc3)cc(-c3ccccc3)c2)c1)c1ccc2c3ccccc3n(-c3ccccc3)c2c1 ; c1ccc(-c2ccc(-c3ccccc3N(c3ccc4c5ccccc5n(-c5ccccc5)c4c3)c3ccc(-c4ccccc4)c4ccccc34)cc2)cc1 ; c1ccc(-c2ccc(-c3ccccc3N(c3ccc4c5ccccc5n(-c5ccccc5)c4c3)c3c(-c4ccccc4)ccc4ccccc34)cc2)cc1]

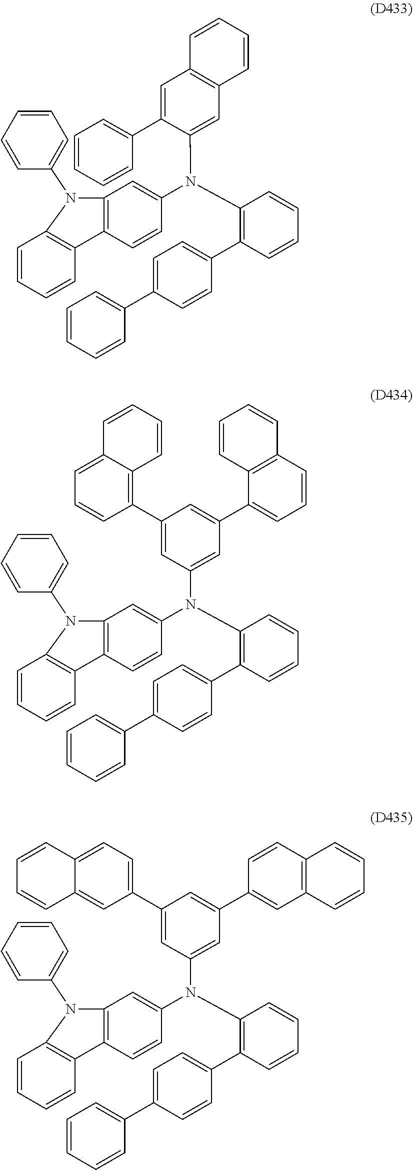 [SMILES: c1ccc(-c2ccc(-c3ccccc3N(c3ccc4c5ccccc5n(-c5ccccc5)c4c3)c3cc4ccccc4cc3-c3ccccc3)cc2)cc1 ; c1ccc(-c2ccc(-c3ccccc3N(c3cc(-c4cccc5ccccc45)cc(-c4cccc5ccccc45)c3)c3ccc4c5ccccc5n(-c5ccccc5)c4c3)cc2)cc1 ; c1ccc(-c2ccc(-c3ccccc3N(c3cc(-c4ccc5ccccc5c4)cc(-c4ccc5ccccc5c4)c3)c3ccc4c5ccccc5n(-c5ccccc5)c4c3)cc2)cc1]

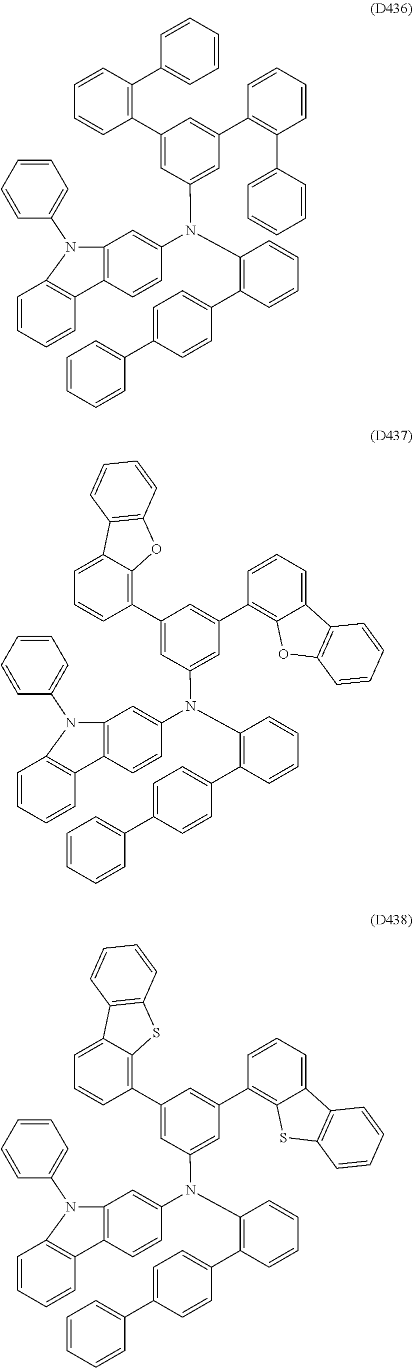 [SMILES: c1ccc(-c2ccc(-c3ccccc3N(c3cc(-c4ccccc4-c4ccccc4)cc(-c4ccccc4-c4ccccc4)c3)c3ccc4c5ccccc5n(-c5ccccc5)c4c3)cc2)cc1 ; c1ccc(-c2ccc(-c3ccccc3N(c3cc(-c4cccc5c4oc4ccccc45)cc(-c4cccc5c4oc4ccccc45)c3)c3ccc4c5ccccc5n(-c5ccccc5)c4c3)cc2)cc1 ; c1ccc(-c2ccc(-c3ccccc3N(c3cc(-c4cccc5c4sc4ccccc45)cc(-c4cccc5c4sc4ccccc45)c3)c3ccc4c5ccccc5n(-c5ccccc5)c4c3)cc2)cc1]

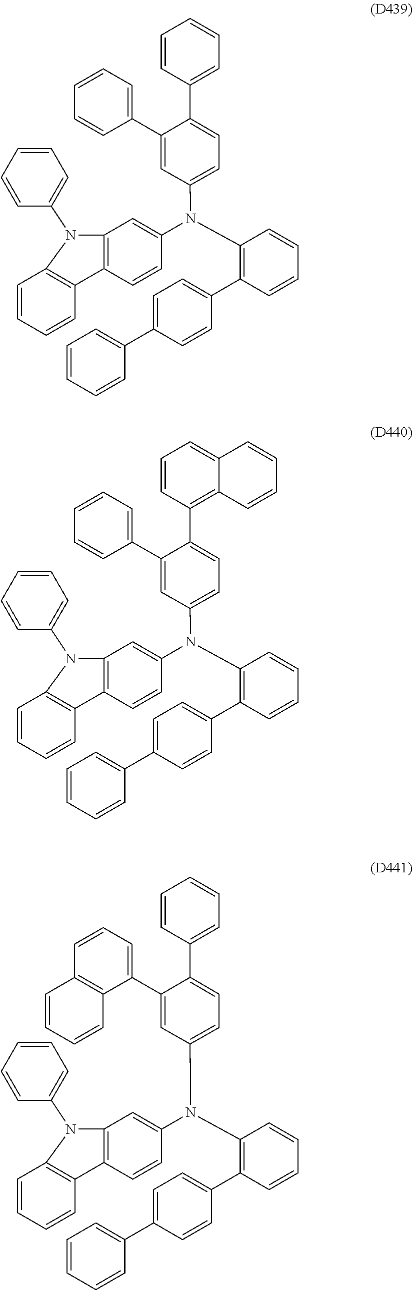 [SMILES: c1ccc(-c2ccc(-c3ccccc3N(c3ccc(-c4ccccc4)c(-c4ccccc4)c3)c3ccc4c5ccccc5n(-c5ccccc5)c4c3)cc2)cc1 ; c1ccc(-c2ccc(-c3ccccc3N(c3ccc(-c4cccc5ccccc45)c(-c4ccccc4)c3)c3ccc4c5ccccc5n(-c5ccccc5)c4c3)cc2)cc1 ; c1ccc(-c2ccc(-c3ccccc3N(c3ccc(-c4ccccc4)c(-c4cccc5ccccc45)c3)c3ccc4c5ccccc5n(-c5ccccc5)c4c3)cc2)cc1]

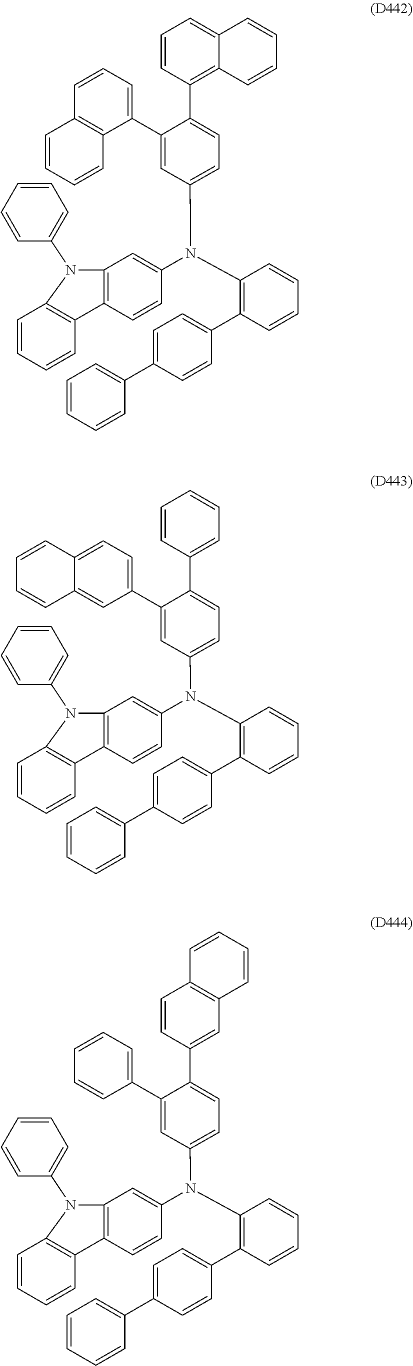 [SMILES: c1ccc(-c2ccc(-c3ccccc3N(c3ccc(-c4cccc5ccccc45)c(-c4cccc5ccccc45)c3)c3ccc4c5ccccc5n(-c5ccccc5)c4c3)cc2)cc1 ; c1ccc(-c2ccc(-c3ccccc3N(c3ccc(-c4ccccc4)c(-c4ccc5ccccc5c4)c3)c3ccc4c5ccccc5n(-c5ccccc5)c4c3)cc2)cc1 ; c1ccc(-c2ccc(-c3ccccc3N(c3ccc(-c4ccc5ccccc5c4)c(-c4ccccc4)c3)c3ccc4c5ccccc5n(-c5ccccc5)c4c3)cc2)cc1]

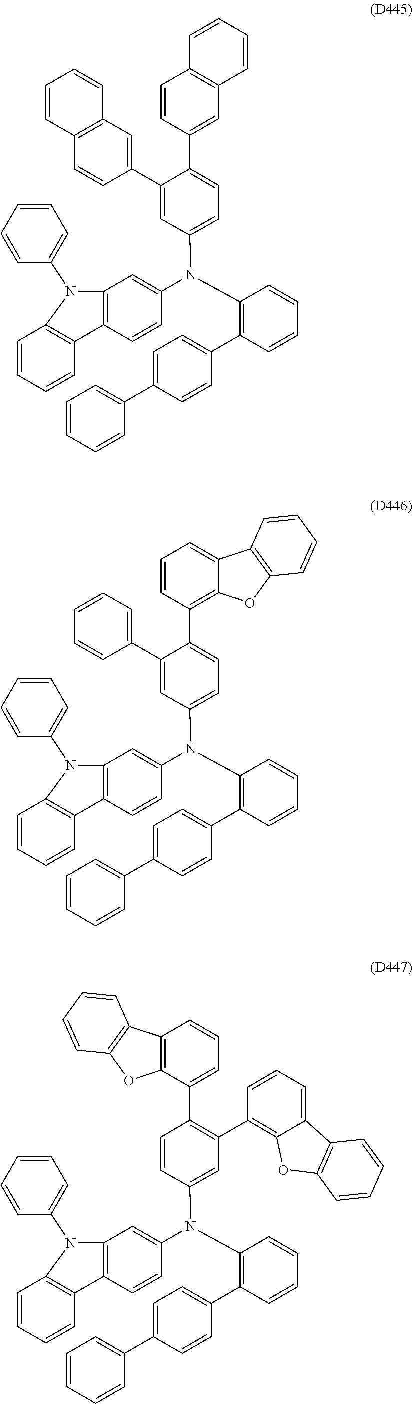 [SMILES: c1ccc(-c2ccc(-c3ccccc3N(c3ccc(-c4ccc5ccccc5c4)c(-c4ccc5ccccc5c4)c3)c3ccc4c5ccccc5n(-c5ccccc5)c4c3)cc2)cc1 ; c1ccc(-c2ccc(-c3ccccc3N(c3ccc(-c4cccc5c4oc4ccccc45)c(-c4ccccc4)c3)c3ccc4c5ccccc5n(-c5ccccc5)c4c3)cc2)cc1 ; c1ccc(-c2ccc(-c3ccccc3N(c3ccc(-c4cccc5c4oc4ccccc45)c(-c4cccc5c4oc4ccccc45)c3)c3ccc4c5ccccc5n(-c5ccccc5)c4c3)cc2)cc1]

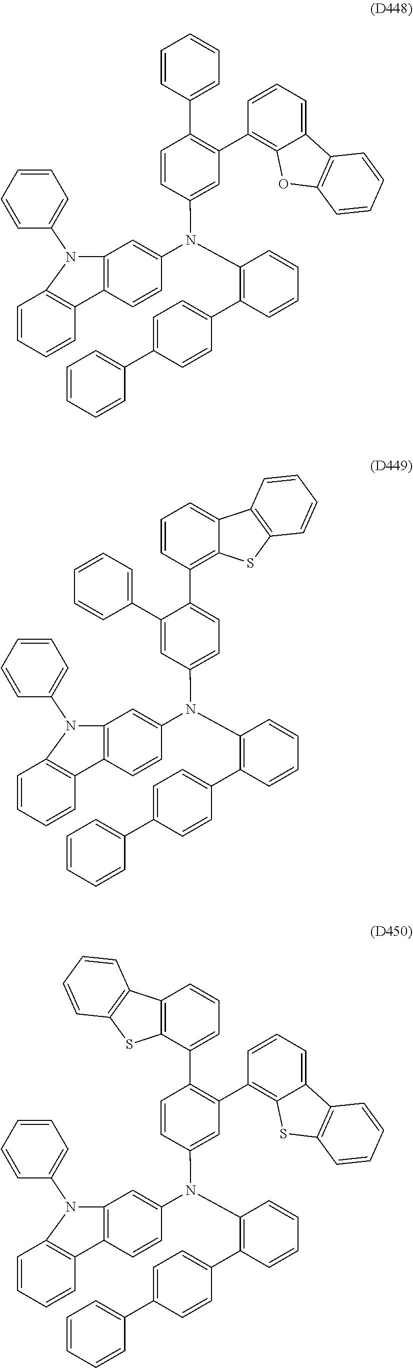 [SMILES: c1ccc(-c2ccc(-c3ccccc3N(c3ccc(-c4ccccc4)c(-c4cccc5c4oc4ccccc45)c3)c3ccc4c5ccccc5n(-c5ccccc5)c4c3)cc2)cc1 ; c1ccc(-c2ccc(-c3ccccc3N(c3ccc(-c4cccc5c4sc4ccccc45)c(-c4ccccc4)c3)c3ccc4c5ccccc5n(-c5ccccc5)c4c3)cc2)cc1 ; c1ccc(-c2ccc(-c3ccccc3N(c3ccc(-c4cccc5c4sc4ccccc45)c(-c4cccc5c4sc4ccccc45)c3)c3ccc4c5ccccc5n(-c5ccccc5)c4c3)cc2)cc1]

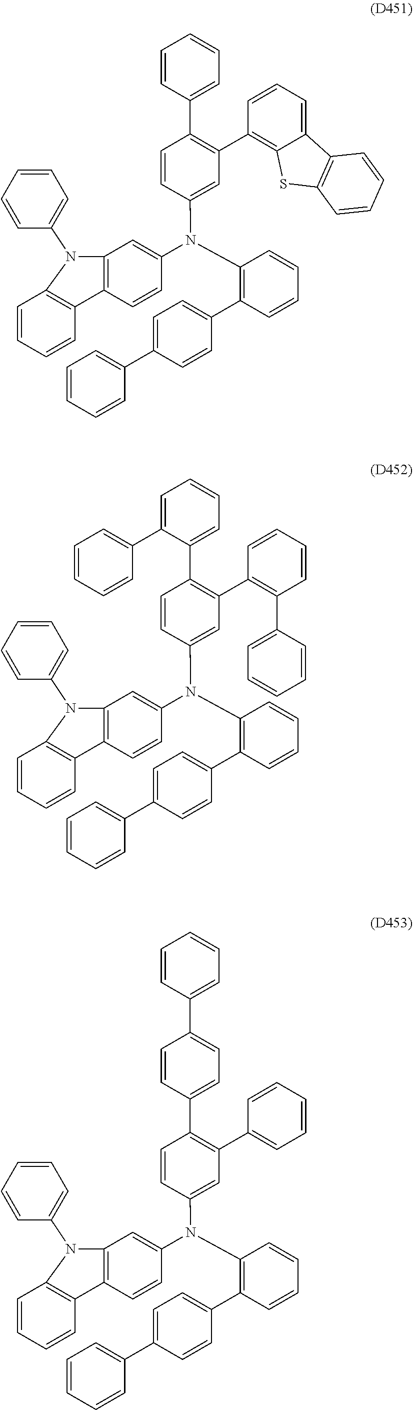 [SMILES: c1ccc(-c2ccc(-c3ccccc3N(c3ccc(-c4ccccc4)c(-c4cccc5c4sc4ccccc45)c3)c3ccc4c5ccccc5n(-c5ccccc5)c4c3)cc2)cc1 ; c1ccc(-c2ccc(-c3ccccc3N(c3ccc(-c4ccccc4-c4ccccc4)c(-c4ccccc4-c4ccccc4)c3)c3ccc4c5ccccc5n(-c5ccccc5)c4c3)cc2)cc1 ; c1ccc(-c2ccc(-c3ccc(N(c4ccc5c6ccccc6n(-c6ccccc6)c5c4)c4ccccc4-c4ccc(-c5ccccc5)cc4)cc3-c3ccccc3)cc2)cc1]

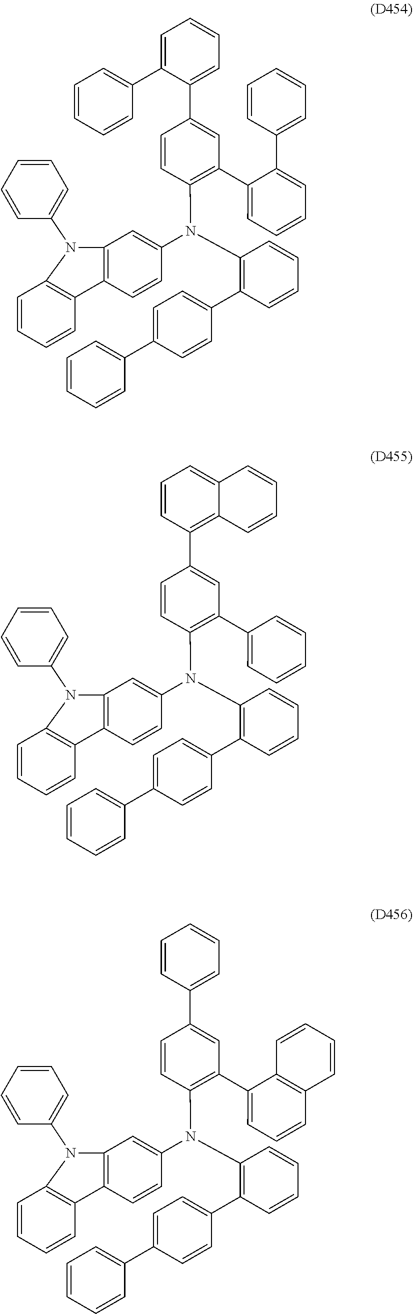 [SMILES: c1ccc(-c2ccc(-c3ccccc3N(c3ccc4c5ccccc5n(-c5ccccc5)c4c3)c3ccc(-c4ccccc4-c4ccccc4)cc3-c3ccccc3-c3ccccc3)cc2)cc1 ; c1ccc(-c2ccc(-c3ccccc3N(c3ccc4c5ccccc5n(-c5ccccc5)c4c3)c3ccc(-c4cccc5ccccc45)cc3-c3ccccc3)cc2)cc1 ; c1ccc(-c2ccc(-c3ccccc3N(c3ccc4c5ccccc5n(-c5ccccc5)c4c3)c3ccc(-c4ccccc4)cc3-c3cccc4ccccc34)cc2)cc1]

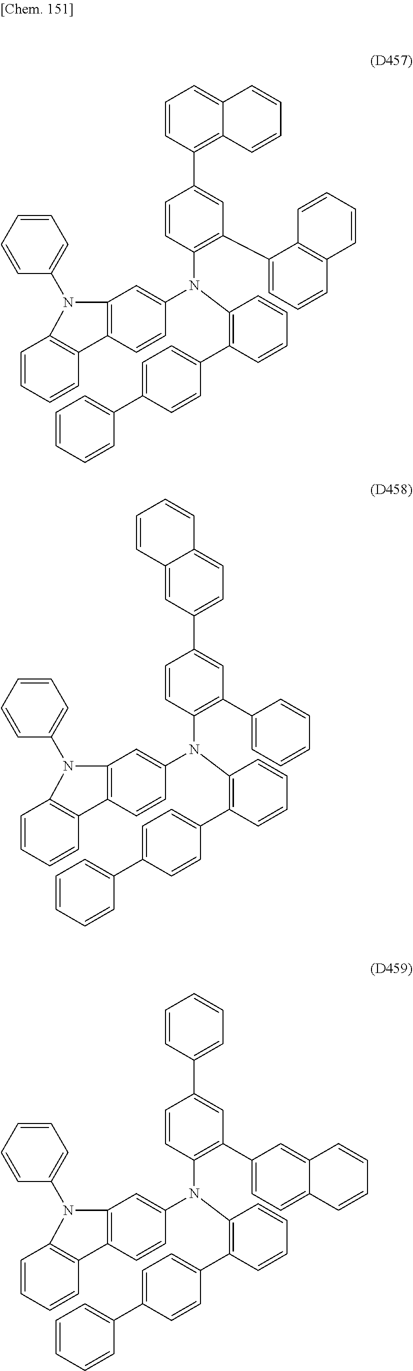 [SMILES: c1ccc(-c2ccc(-c3ccccc3N(c3ccc4c5ccccc5n(-c5ccccc5)c4c3)c3ccc(-c4cccc5ccccc45)cc3-c3cccc4ccccc34)cc2)cc1 ; c1ccc(-c2ccc(-c3ccccc3N(c3ccc4c5ccccc5n(-c5ccccc5)c4c3)c3ccc(-c4ccc5ccccc5c4)cc3-c3ccccc3)cc2)cc1 ; c1ccc(-c2ccc(-c3ccccc3N(c3ccc4c5ccccc5n(-c5ccccc5)c4c3)c3ccc(-c4ccccc4)cc3-c3ccc4ccccc4c3)cc2)cc1]

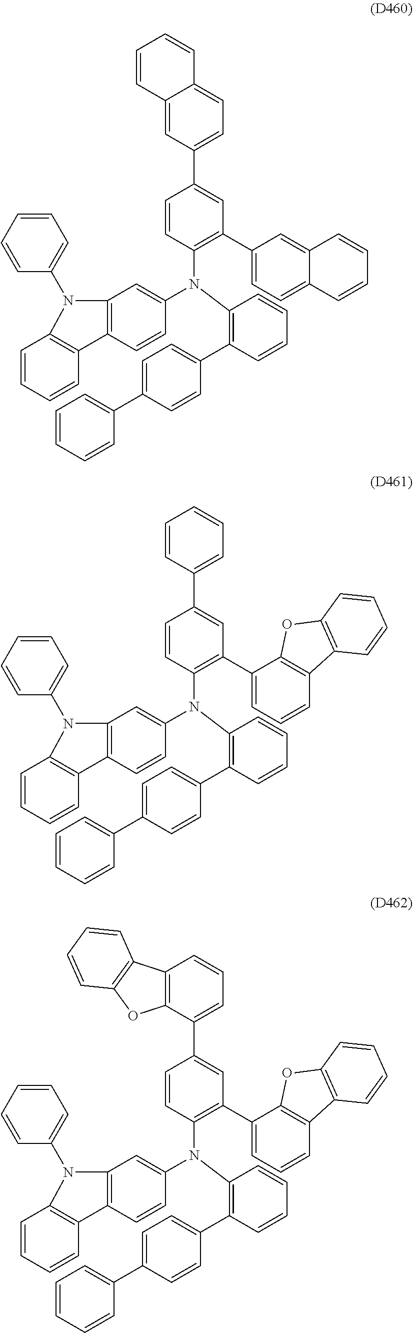

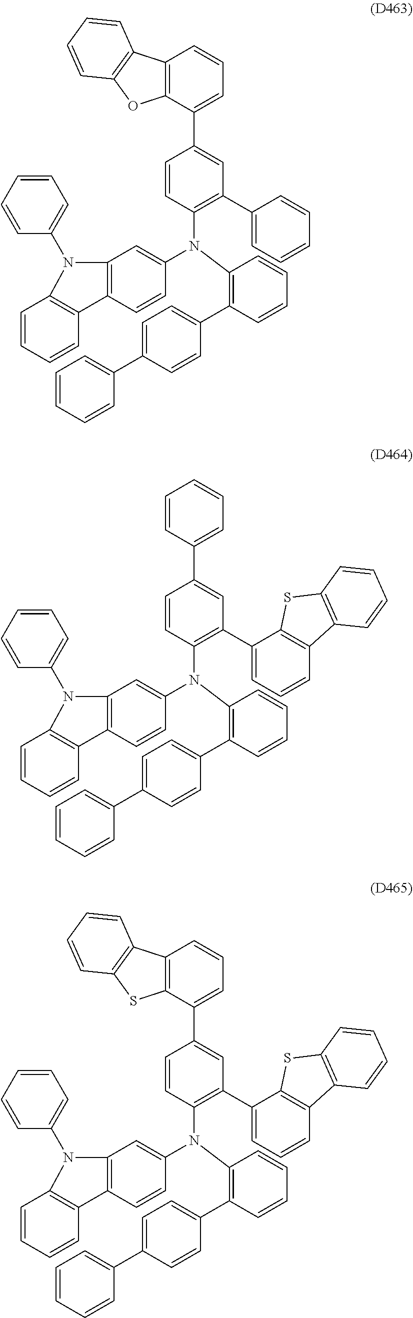 [SMILES: c1ccc(-c2ccc(-c3ccccc3N(c3ccc4c5ccccc5n(-c5ccccc5)c4c3)c3ccc(-c4cccc5c4oc4ccccc45)cc3-c3ccccc3)cc2)cc1 ; c1ccc(-c2ccc(-c3ccccc3N(c3ccc4c5ccccc5n(-c5ccccc5)c4c3)c3ccc(-c4ccccc4)cc3-c3cccc4c3sc3ccccc34)cc2)cc1 ; c1ccc(-c2ccc(-c3ccccc3N(c3ccc4c5ccccc5n(-c5ccccc5)c4c3)c3ccc(-c4cccc5c4sc4ccccc45)cc3-c3cccc4c3sc3ccccc34)cc2)cc1]

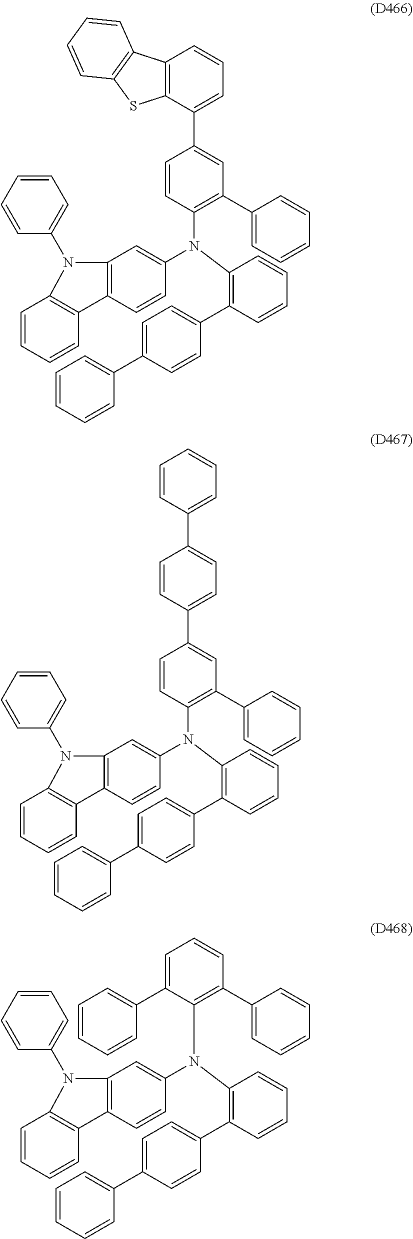 [SMILES: c1ccc(-c2ccc(-c3ccccc3N(c3ccc4c5ccccc5n(-c5ccccc5)c4c3)c3ccc(-c4cccc5c4sc4ccccc45)cc3-c3ccccc3)cc2)cc1 ; c1ccc(-c2ccc(-c3ccc(N(c4ccc5c6ccccc6n(-c6ccccc6)c5c4)c4ccccc4-c4ccc(-c5ccccc5)cc4)c(-c4ccccc4)c3)cc2)cc1 ; c1ccc(-c2ccc(-c3ccccc3N(c3ccc4c5ccccc5n(-c5ccccc5)c4c3)c3c(-c4ccccc4)cccc3-c3ccccc3)cc2)cc1]

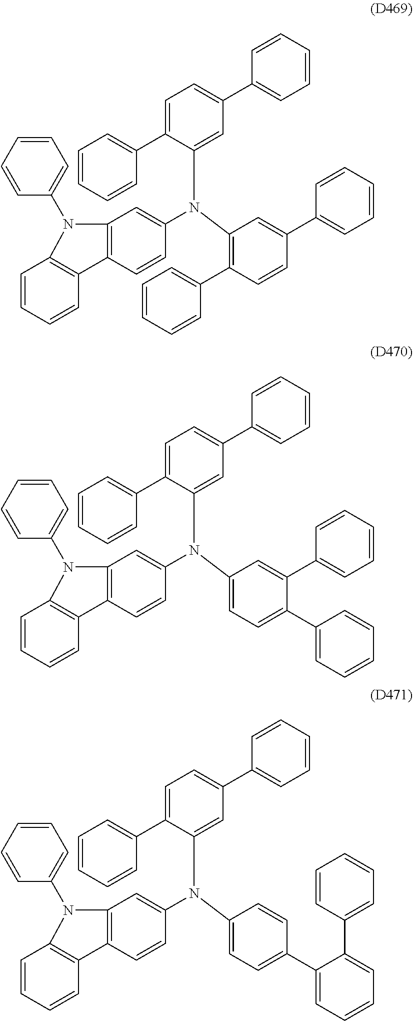 [SMILES: c1ccc(-c2ccc(-c3ccccc3)c(N(c3ccc4c5ccccc5n(-c5ccccc5)c4c3)c3cc(-c4ccccc4)ccc3-c3ccccc3)c2)cc1 ; c1ccc(-c2ccc(-c3ccccc3)c(N(c3ccc(-c4ccccc4)c(-c4ccccc4)c3)c3ccc4c5ccccc5n(-c5ccccc5)c4c3)c2)cc1 ; c1ccc(-c2ccc(-c3ccccc3)c(N(c3ccc(-c4ccccc4-c4ccccc4)cc3)c3ccc4c5ccccc5n(-c5ccccc5)c4c3)c2)cc1]

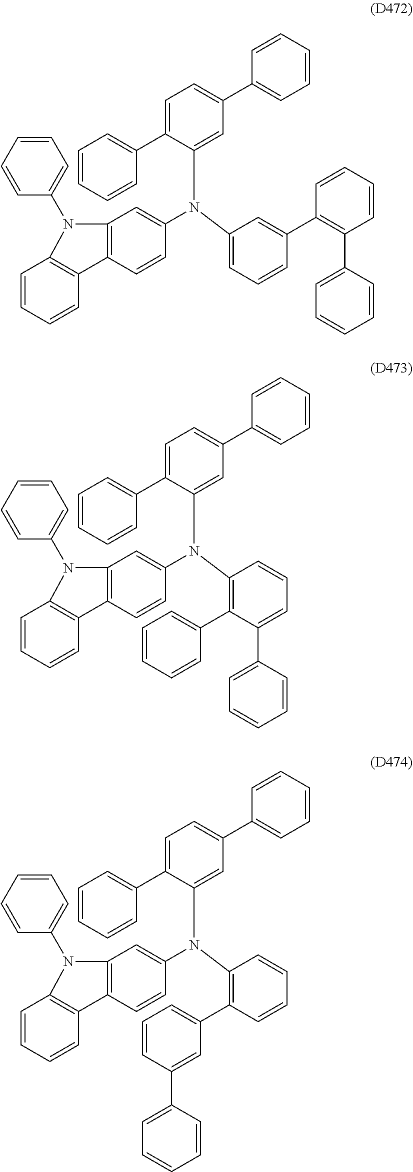 [SMILES: c1ccc(-c2ccc(-c3ccccc3)c(N(c3cccc(-c4ccccc4-c4ccccc4)c3)c3ccc4c5ccccc5n(-c5ccccc5)c4c3)c2)cc1 ; c1ccc(-c2ccc(-c3ccccc3)c(N(c3ccc4c5ccccc5n(-c5ccccc5)c4c3)c3cccc(-c4ccccc4)c3-c3ccccc3)c2)cc1 ; c1ccc(-c2cccc(-c3ccccc3N(c3ccc4c5ccccc5n(-c5ccccc5)c4c3)c3cc(-c4ccccc4)ccc3-c3ccccc3)c2)cc1]

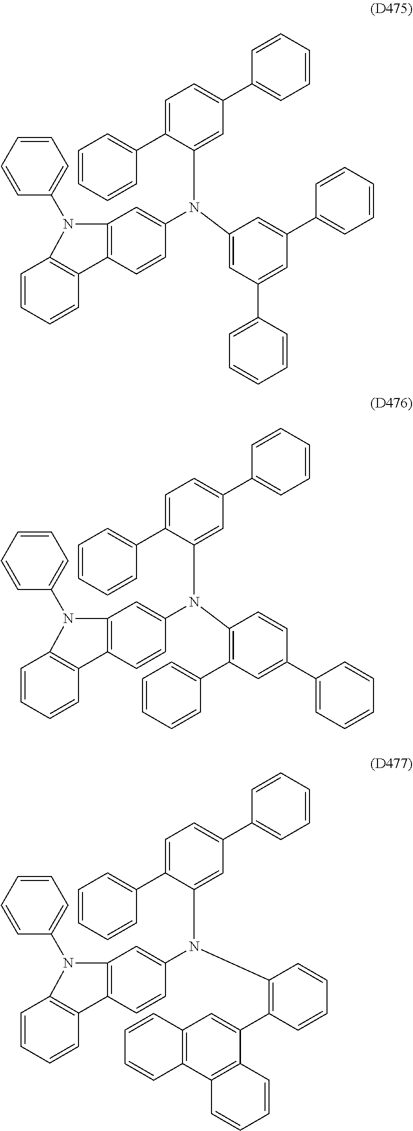 [SMILES: c1ccc(-c2cc(-c3ccccc3)cc(N(c3ccc4c5ccccc5n(-c5ccccc5)c4c3)c3cc(-c4ccccc4)ccc3-c3ccccc3)c2)cc1 ; c1ccc(-c2ccc(N(c3ccc4c5ccccc5n(-c5ccccc5)c4c3)c3cc(-c4ccccc4)ccc3-c3ccccc3)c(-c3ccccc3)c2)cc1 ; c1ccc(-c2ccc(-c3ccccc3)c(N(c3ccc4c5ccccc5n(-c5ccccc5)c4c3)c3ccccc3-c3cc4ccccc4c4ccccc34)c2)cc1]

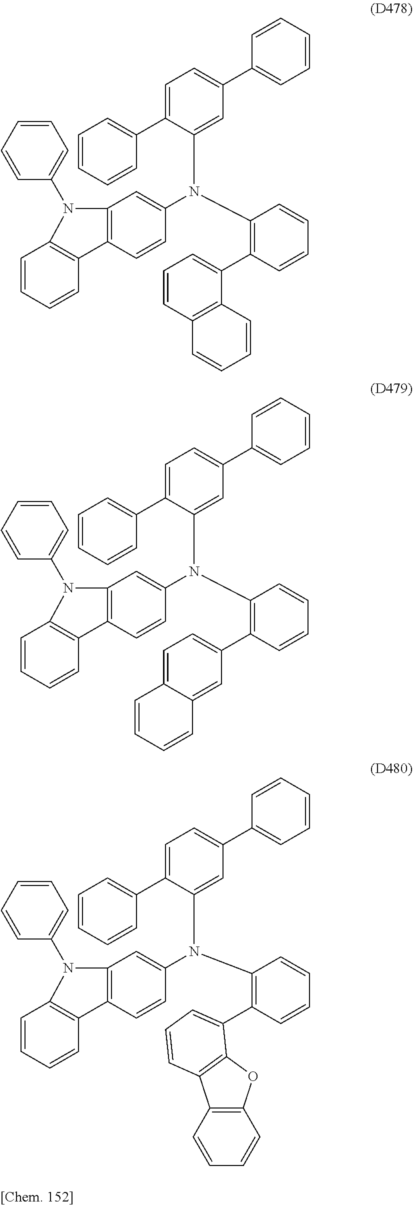 [SMILES: c1ccc(-c2ccc(-c3ccccc3)c(N(c3ccc4c5ccccc5n(-c5ccccc5)c4c3)c3ccccc3-c3cccc4ccccc34)c2)cc1 ; c1ccc(-c2ccc(-c3ccccc3)c(N(c3ccc4c5ccccc5n(-c5ccccc5)c4c3)c3ccccc3-c3ccc4ccccc4c3)c2)cc1 ; c1ccc(-c2ccc(-c3ccccc3)c(N(c3ccc4c5ccccc5n(-c5ccccc5)c4c3)c3ccccc3-c3cccc4c3oc3ccccc34)c2)cc1]

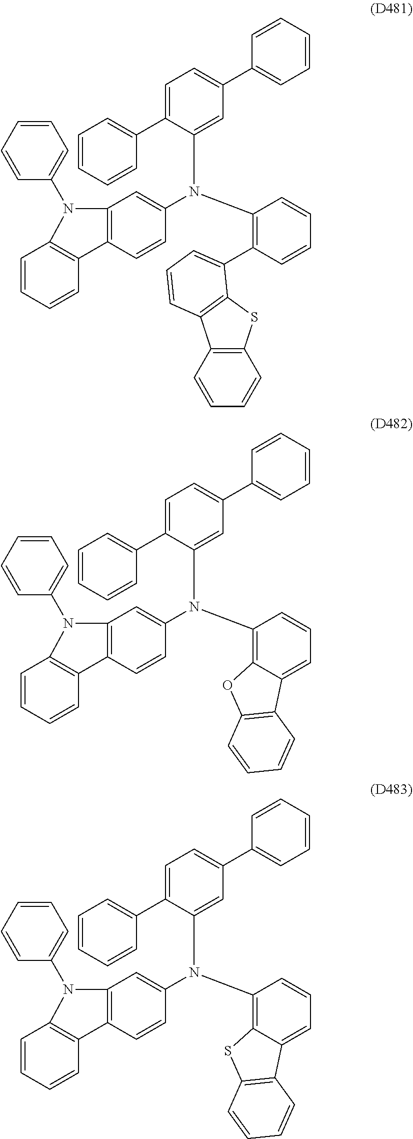

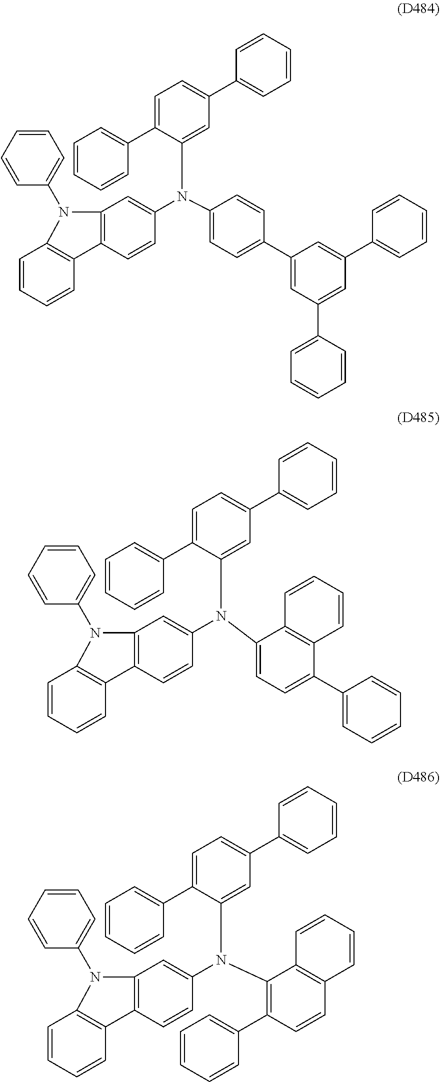

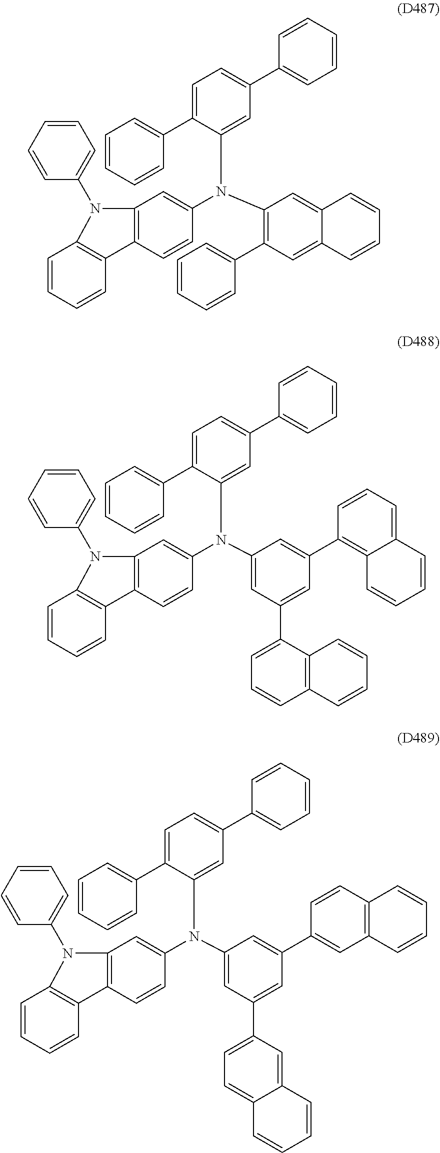 [SMILES: c1ccc(-c2ccc(-c3ccccc3)c(N(c3ccc4c5ccccc5n(-c5ccccc5)c4c3)c3cc4ccccc4cc3-c3ccccc3)c2)cc1 ; c1ccc(-c2ccc(-c3ccccc3)c(N(c3cc(-c4cccc5ccccc45)cc(-c4cccc5ccccc45)c3)c3ccc4c5ccccc5n(-c5ccccc5)c4c3)c2)cc1 ; c1ccc(-c2ccc(-c3ccccc3)c(N(c3cc(-c4ccc5ccccc5c4)cc(-c4ccc5ccccc5c4)c3)c3ccc4c5ccccc5n(-c5ccccc5)c4c3)c2)cc1]

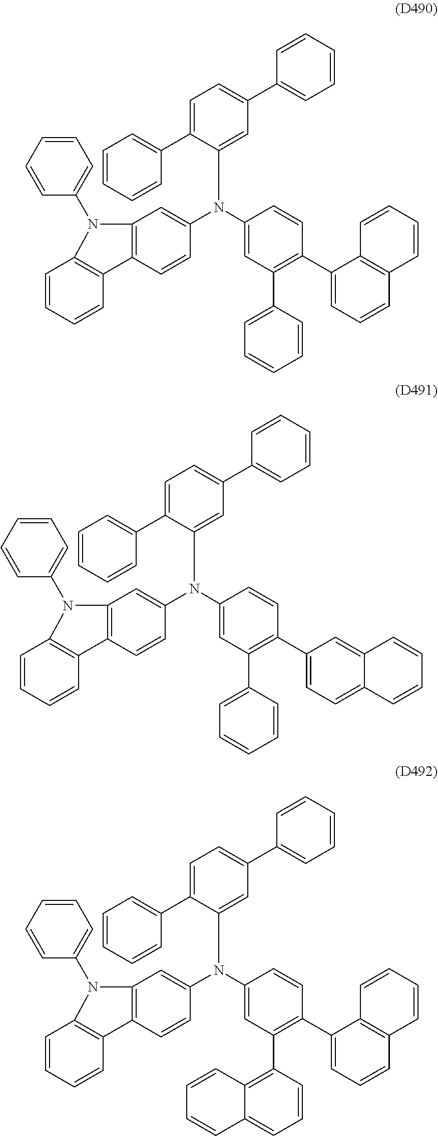

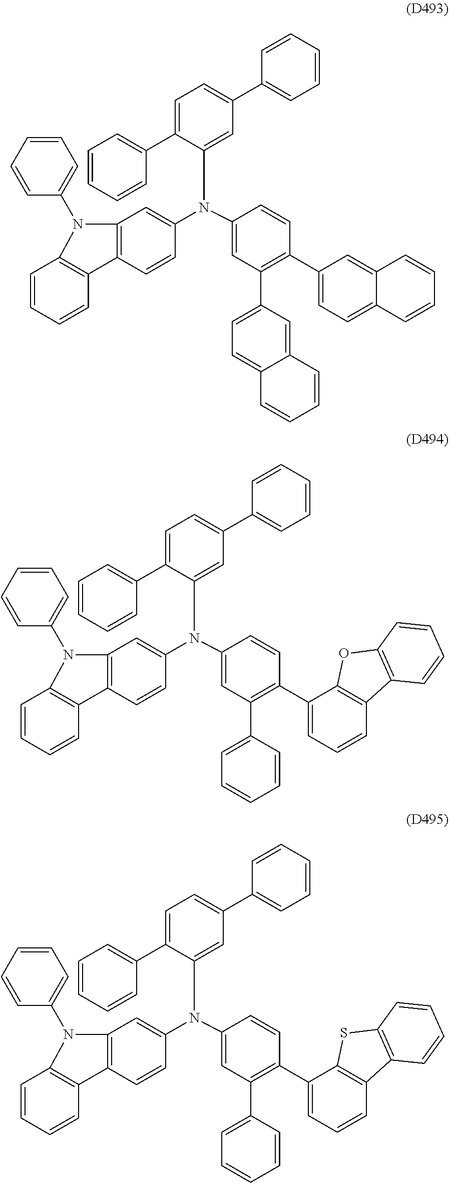

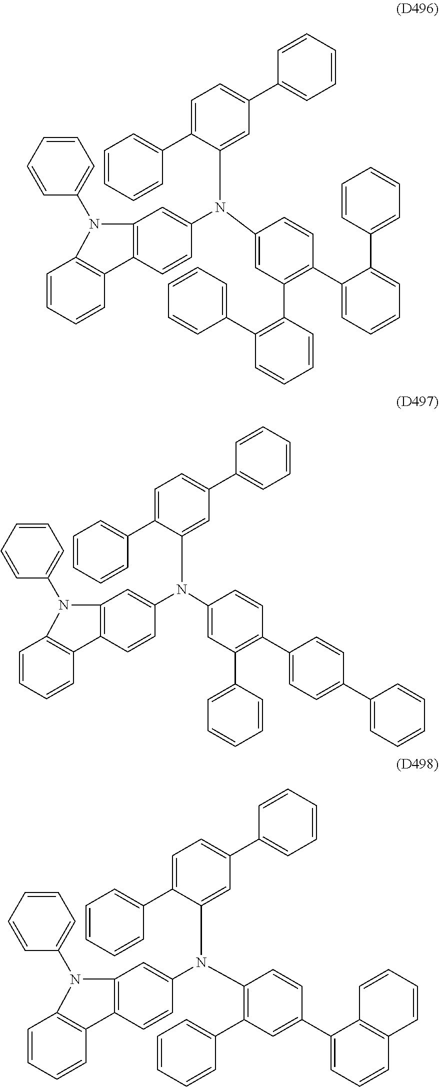 [SMILES: c1ccc(-c2ccc(-c3ccccc3)c(N(c3ccc(-c4ccccc4-c4ccccc4)c(-c4ccccc4-c4ccccc4)c3)c3ccc4c5ccccc5n(-c5ccccc5)c4c3)c2)cc1 ; c1ccc(-c2ccc(-c3ccc(N(c4ccc5c6ccccc6n(-c6ccccc6)c5c4)c4cc(-c5ccccc5)ccc4-c4ccccc4)cc3-c3ccccc3)cc2)cc1 ; c1ccc(-c2ccc(-c3ccccc3)c(N(c3ccc4c5ccccc5n(-c5ccccc5)c4c3)c3ccc(-c4cccc5ccccc45)cc3-c3ccccc3)c2)cc1]

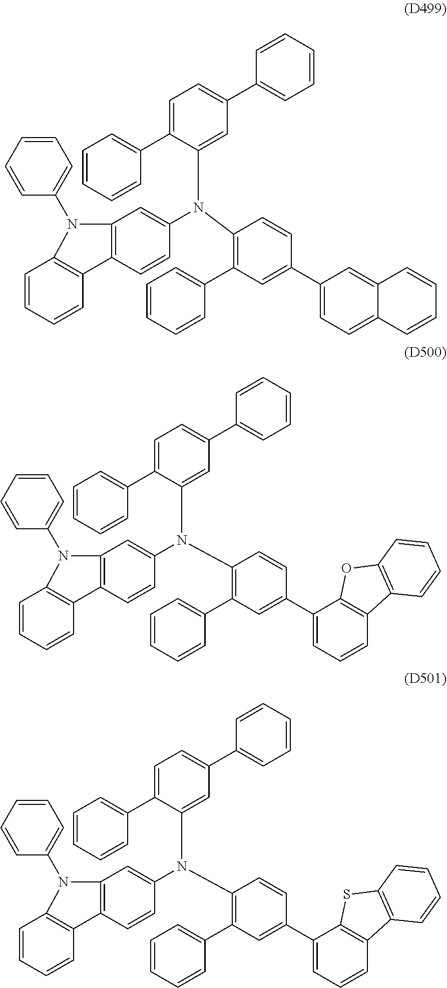 [SMILES: c1ccc(-c2ccc(-c3ccccc3)c(N(c3ccc4c5ccccc5n(-c5ccccc5)c4c3)c3ccc(-c4ccc5ccccc5c4)cc3-c3ccccc3)c2)cc1 ; c1ccc(-c2ccc(-c3ccccc3)c(N(c3ccc4c5ccccc5n(-c5ccccc5)c4c3)c3ccc(-c4cccc5c4oc4ccccc45)cc3-c3ccccc3)c2)cc1 ; c1ccc(-c2ccc(-c3ccccc3)c(N(c3ccc4c5ccccc5n(-c5ccccc5)c4c3)c3ccc(-c4cccc5c4sc4ccccc45)cc3-c3ccccc3)c2)cc1]

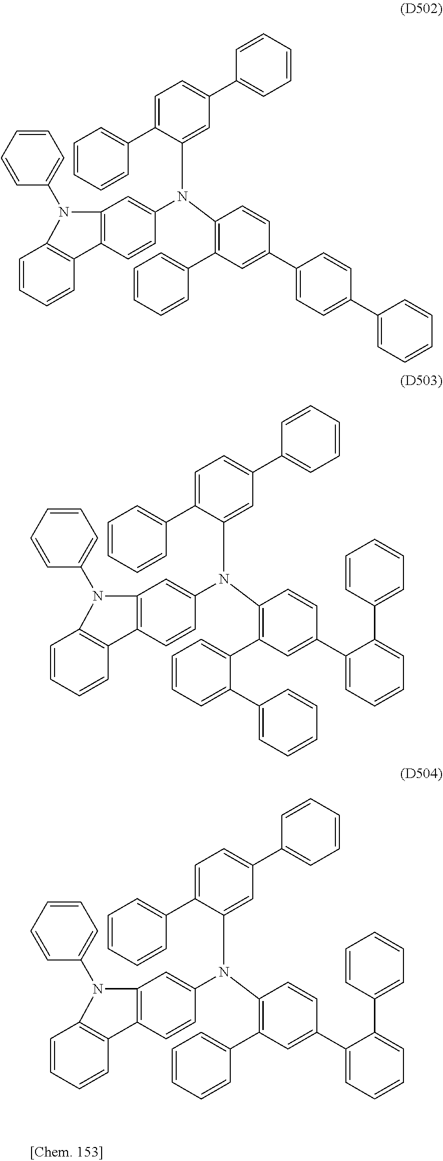 [SMILES: c1ccc(-c2ccc(-c3ccc(N(c4ccc5c6ccccc6n(-c6ccccc6)c5c4)c4cc(-c5ccccc5)ccc4-c4ccccc4)c(-c4ccccc4)c3)cc2)cc1 ; c1ccc(-c2ccc(-c3ccccc3)c(N(c3ccc4c5ccccc5n(-c5ccccc5)c4c3)c3ccc(-c4ccccc4-c4ccccc4)cc3-c3ccccc3-c3ccccc3)c2)cc1 ; c1ccc(-c2ccc(-c3ccccc3)c(N(c3ccc4c5ccccc5n(-c5ccccc5)c4c3)c3ccc(-c4ccccc4-c4ccccc4)cc3-c3ccccc3)c2)cc1]

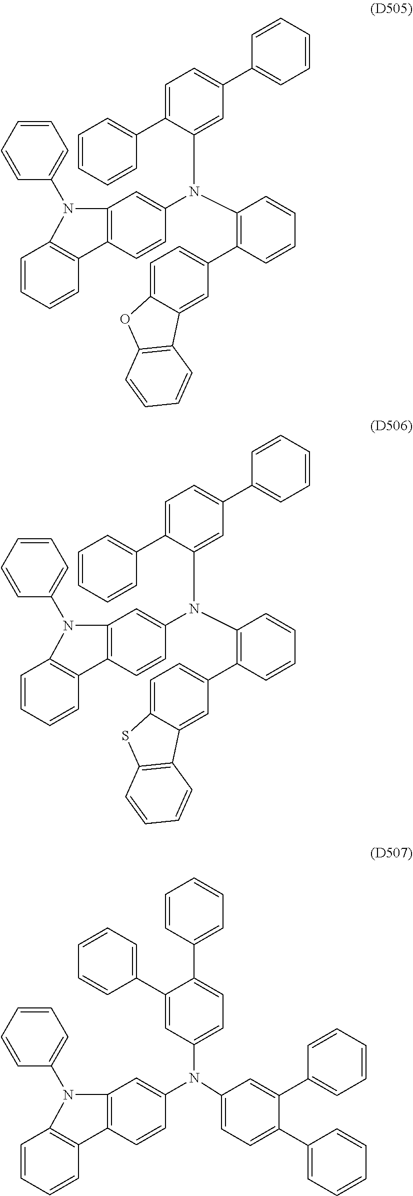 [SMILES: c1ccc(-c2ccc(-c3ccccc3)c(N(c3ccc4c5ccccc5n(-c5ccccc5)c4c3)c3ccccc3-c3ccc4oc5ccccc5c4c3)c2)cc1 ; c1ccc(-c2ccc(-c3ccccc3)c(N(c3ccc4c5ccccc5n(-c5ccccc5)c4c3)c3ccccc3-c3ccc4sc5ccccc5c4c3)c2)cc1 ; c1ccc(-c2ccc(N(c3ccc(-c4ccccc4)c(-c4ccccc4)c3)c3ccc4c5ccccc5n(-c5ccccc5)c4c3)cc2-c2ccccc2)cc1]

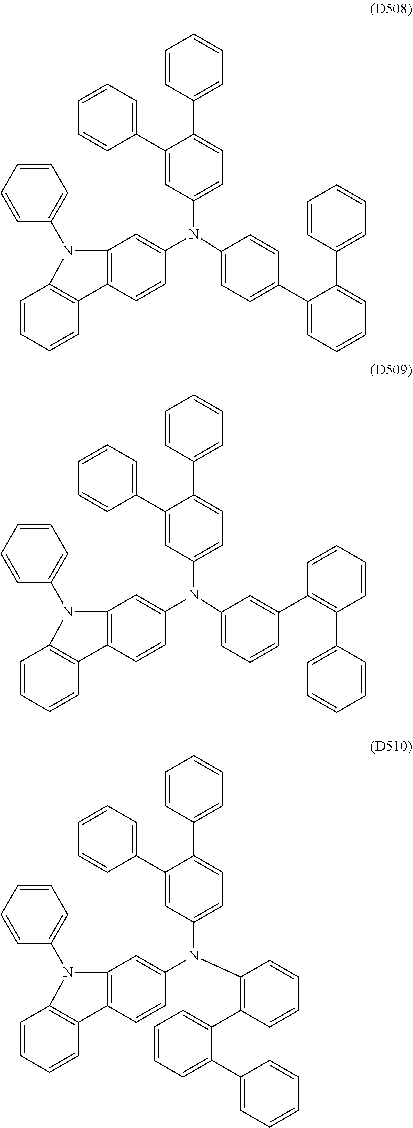 [SMILES: c1ccc(-c2ccccc2-c2ccc(N(c3ccc(-c4ccccc4)c(-c4ccccc4)c3)c3ccc4c5ccccc5n(-c5ccccc5)c4c3)cc2)cc1 ; c1ccc(-c2ccccc2-c2cccc(N(c3ccc(-c4ccccc4)c(-c4ccccc4)c3)c3ccc4c5ccccc5n(-c5ccccc5)c4c3)c2)cc1 ; c1ccc(-c2ccc(N(c3ccc4c5ccccc5n(-c5ccccc5)c4c3)c3ccccc3-c3ccccc3-c3ccccc3)cc2-c2ccccc2)cc1]

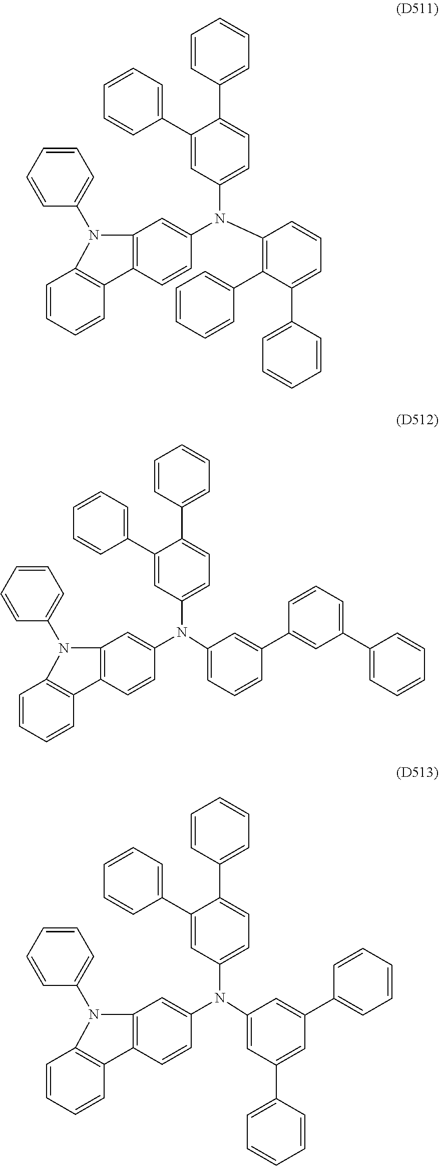 [SMILES: c1ccc(-c2ccc(N(c3ccc4c5ccccc5n(-c5ccccc5)c4c3)c3cccc(-c4ccccc4)c3-c3ccccc3)cc2-c2ccccc2)cc1 ; c1ccc(-c2cccc(-c3cccc(N(c4ccc(-c5ccccc5)c(-c5ccccc5)c4)c4ccc5c6ccccc6n(-c6ccccc6)c5c4)c3)c2)cc1 ; c1ccc(-c2cc(-c3ccccc3)cc(N(c3ccc(-c4ccccc4)c(-c4ccccc4)c3)c3ccc4c5ccccc5n(-c5ccccc5)c4c3)c2)cc1]

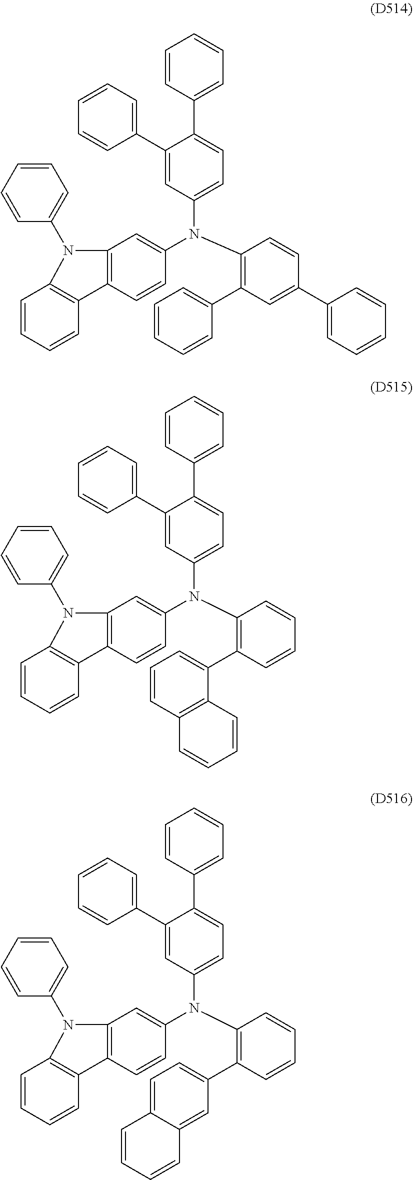

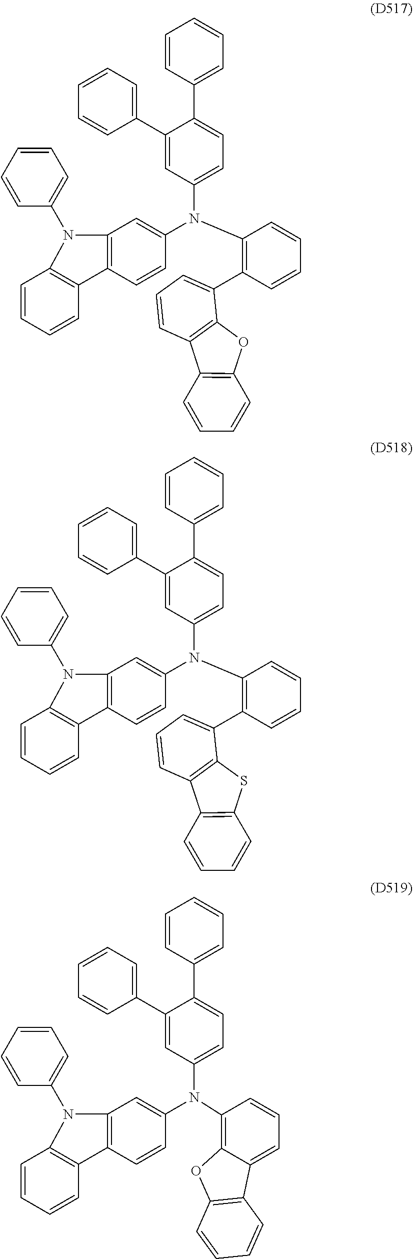

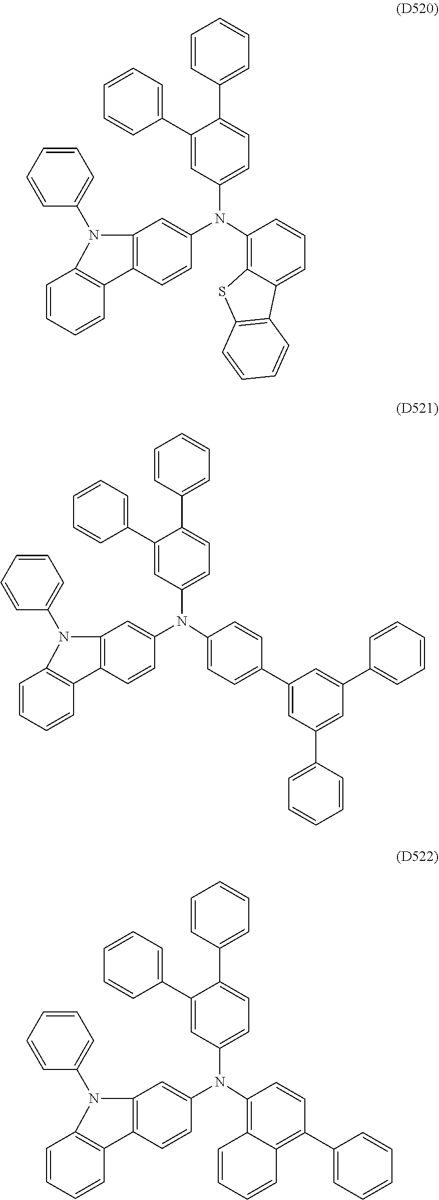 [SMILES: c1ccc(-c2ccc(N(c3ccc4c5ccccc5n(-c5ccccc5)c4c3)c3cccc4c3sc3ccccc34)cc2-c2ccccc2)cc1 ; c1ccc(-c2cc(-c3ccccc3)cc(-c3ccc(N(c4ccc(-c5ccccc5)c(-c5ccccc5)c4)c4ccc5c6ccccc6n(-c6ccccc6)c5c4)cc3)c2)cc1 ; c1ccc(-c2ccc(N(c3ccc4c5ccccc5n(-c5ccccc5)c4c3)c3ccc(-c4ccccc4)c4ccccc34)cc2-c2ccccc2)cc1]

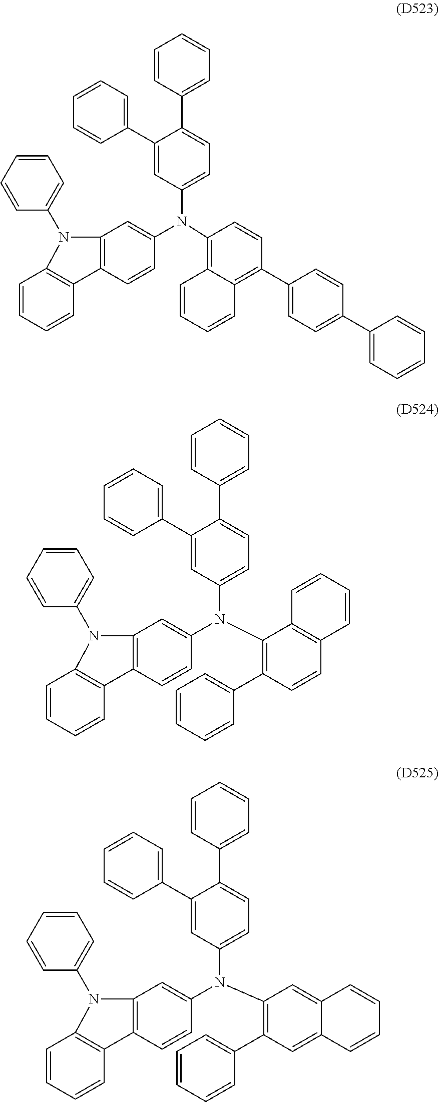

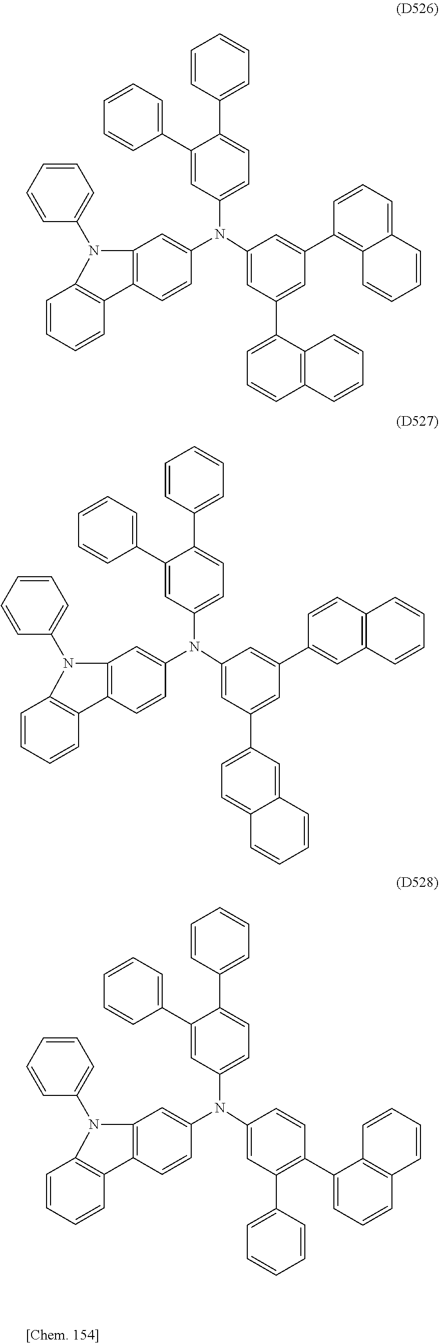 [SMILES: c1ccc(-c2ccc(N(c3cc(-c4cccc5ccccc45)cc(-c4cccc5ccccc45)c3)c3ccc4c5ccccc5n(-c5ccccc5)c4c3)cc2-c2ccccc2)cc1 ; c1ccc(-c2ccc(N(c3cc(-c4ccc5ccccc5c4)cc(-c4ccc5ccccc5c4)c3)c3ccc4c5ccccc5n(-c5ccccc5)c4c3)cc2-c2ccccc2)cc1 ; c1ccc(-c2ccc(N(c3ccc(-c4cccc5ccccc45)c(-c4ccccc4)c3)c3ccc4c5ccccc5n(-c5ccccc5)c4c3)cc2-c2ccccc2)cc1]

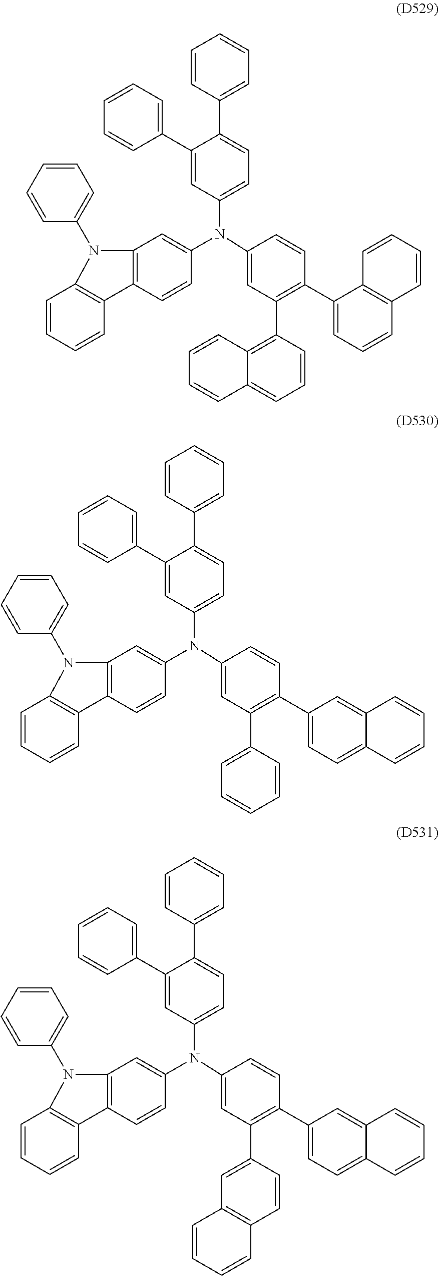 [SMILES: c1ccc(-c2ccc(N(c3ccc(-c4cccc5ccccc45)c(-c4cccc5ccccc45)c3)c3ccc4c5ccccc5n(-c5ccccc5)c4c3)cc2-c2ccccc2)cc1 ; c1ccc(-c2ccc(N(c3ccc(-c4ccc5ccccc5c4)c(-c4ccccc4)c3)c3ccc4c5ccccc5n(-c5ccccc5)c4c3)cc2-c2ccccc2)cc1 ; c1ccc(-c2ccc(N(c3ccc(-c4ccc5ccccc5c4)c(-c4ccc5ccccc5c4)c3)c3ccc4c5ccccc5n(-c5ccccc5)c4c3)cc2-c2ccccc2)cc1]

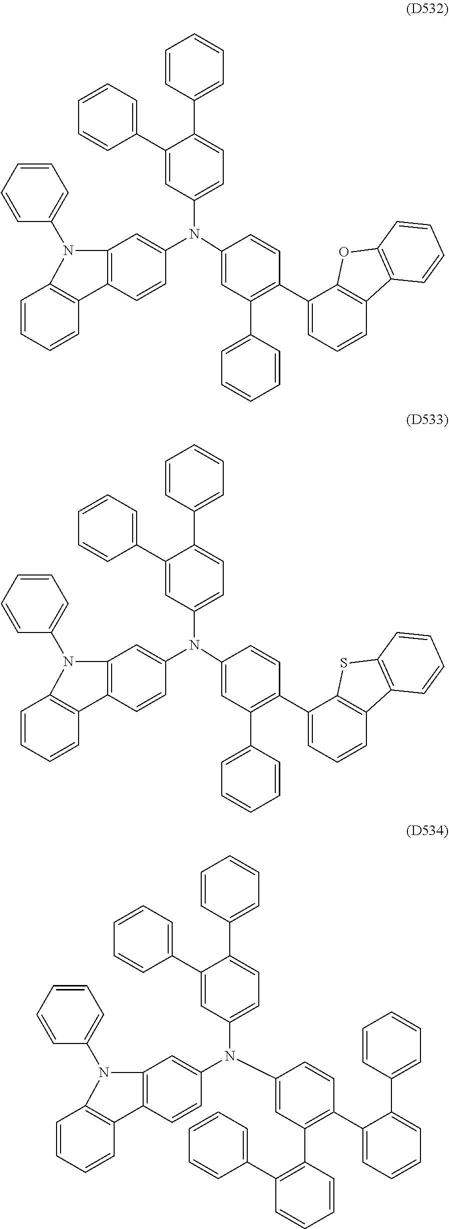 [SMILES: c1ccc(-c2ccc(N(c3ccc(-c4cccc5c4oc4ccccc45)c(-c4ccccc4)c3)c3ccc4c5ccccc5n(-c5ccccc5)c4c3)cc2-c2ccccc2)cc1 ; c1ccc(-c2ccc(N(c3ccc(-c4cccc5c4sc4ccccc45)c(-c4ccccc4)c3)c3ccc4c5ccccc5n(-c5ccccc5)c4c3)cc2-c2ccccc2)cc1 ; c1ccc(-c2ccc(N(c3ccc(-c4ccccc4-c4ccccc4)c(-c4ccccc4-c4ccccc4)c3)c3ccc4c5ccccc5n(-c5ccccc5)c4c3)cc2-c2ccccc2)cc1]

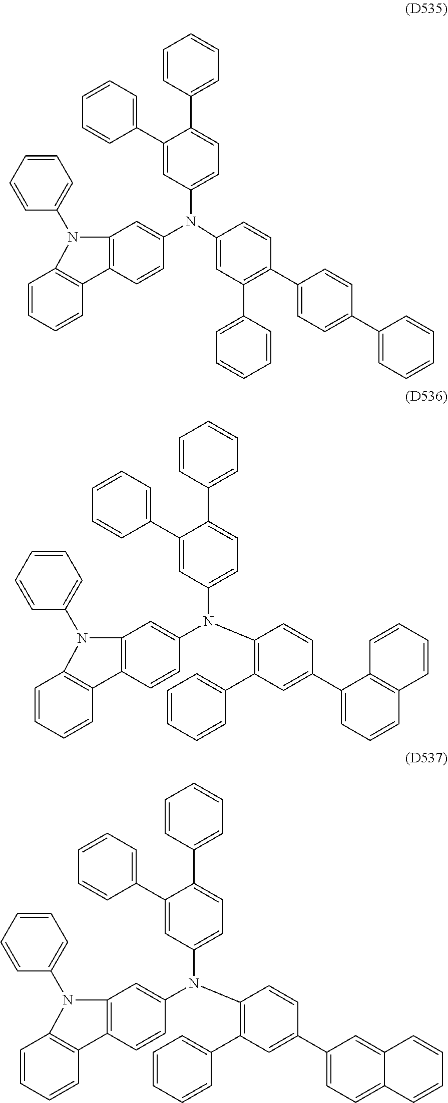

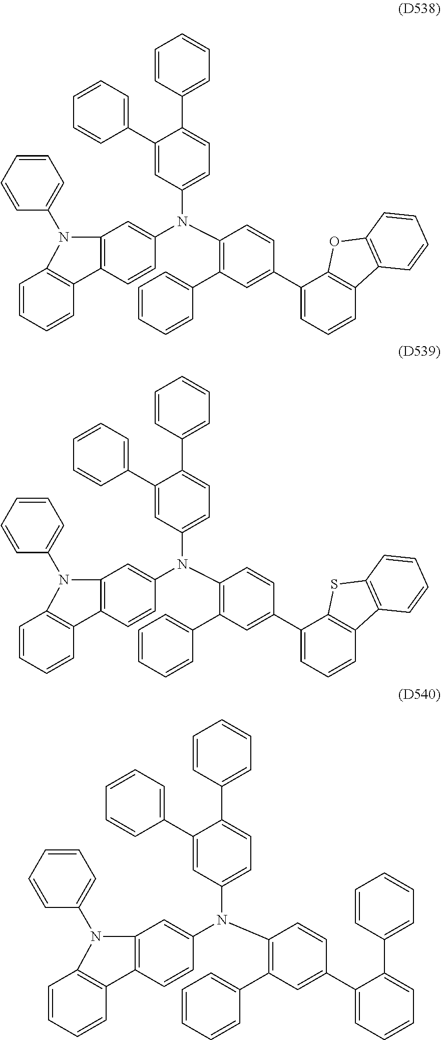 [SMILES: c1ccc(-c2ccc(N(c3ccc4c5ccccc5n(-c5ccccc5)c4c3)c3ccc(-c4cccc5c4oc4ccccc45)cc3-c3ccccc3)cc2-c2ccccc2)cc1 ; c1ccc(-c2ccc(N(c3ccc4c5ccccc5n(-c5ccccc5)c4c3)c3ccc(-c4cccc5c4sc4ccccc45)cc3-c3ccccc3)cc2-c2ccccc2)cc1 ; c1ccc(-c2ccccc2-c2ccc(N(c3ccc(-c4ccccc4)c(-c4ccccc4)c3)c3ccc4c5ccccc5n(-c5ccccc5)c4c3)c(-c3ccccc3)c2)cc1]

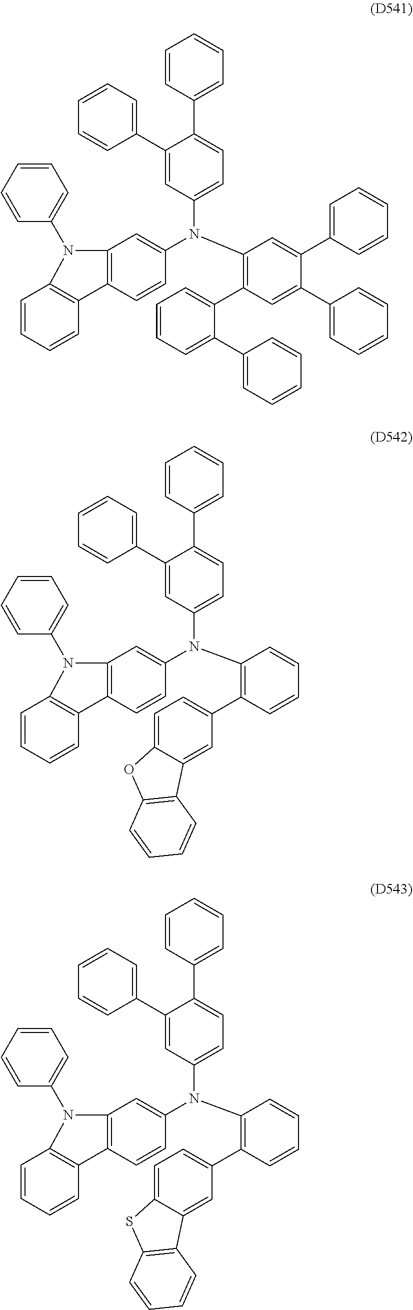

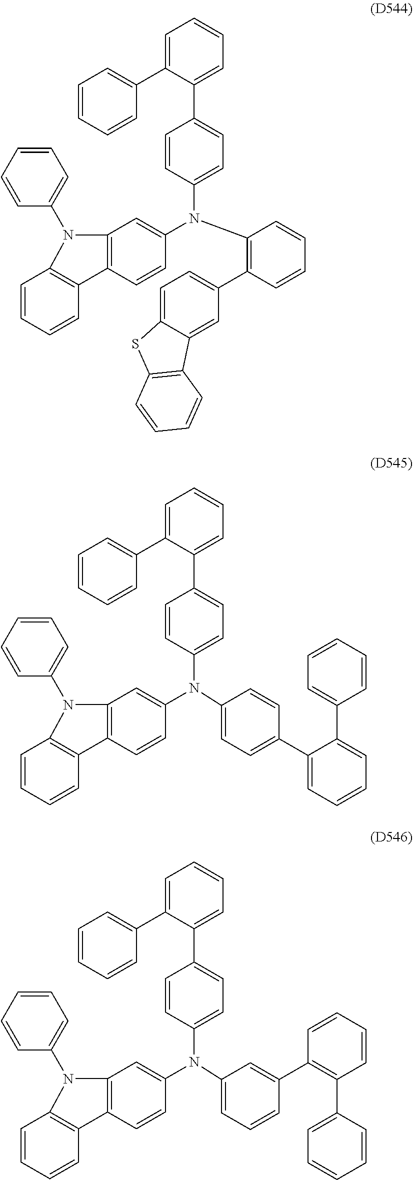 [SMILES: c1ccc(-c2ccccc2-c2ccc(N(c3ccc4c5ccccc5n(-c5ccccc5)c4c3)c3ccccc3-c3ccc4sc5ccccc5c4c3)cc2)cc1 ; c1ccc(-c2ccccc2-c2ccc(N(c3ccc(-c4ccccc4-c4ccccc4)cc3)c3ccc4c5ccccc5n(-c5ccccc5)c4c3)cc2)cc1 ; c1ccc(-c2ccccc2-c2ccc(N(c3cccc(-c4ccccc4-c4ccccc4)c3)c3ccc4c5ccccc5n(-c5ccccc5)c4c3)cc2)cc1]

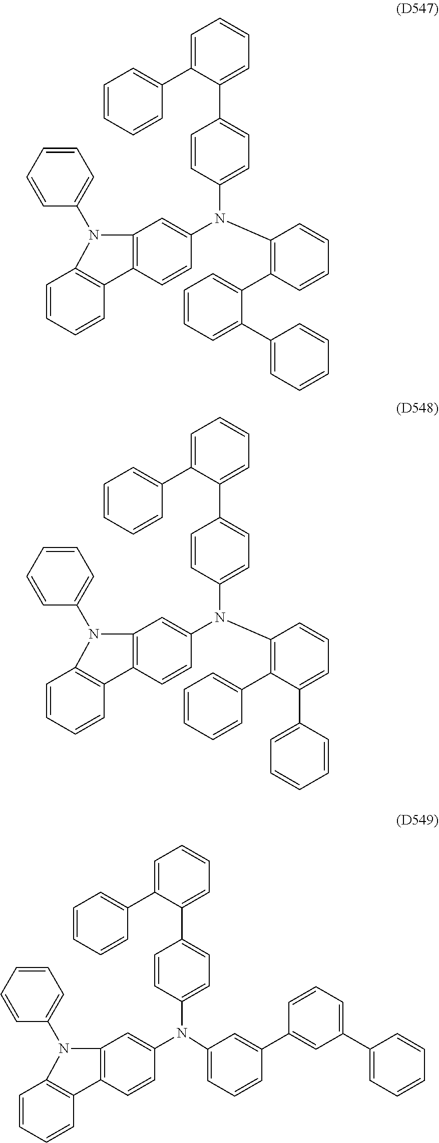 [SMILES: c1ccc(-c2ccccc2-c2ccc(N(c3ccc4c5ccccc5n(-c5ccccc5)c4c3)c3ccccc3-c3ccccc3-c3ccccc3)cc2)cc1 ; c1ccc(-c2ccccc2-c2ccc(N(c3ccc4c5ccccc5n(-c5ccccc5)c4c3)c3cccc(-c4ccccc4)c3-c3ccccc3)cc2)cc1 ; c1ccc(-c2cccc(-c3cccc(N(c4ccc(-c5ccccc5-c5ccccc5)cc4)c4ccc5c6ccccc6n(-c6ccccc6)c5c4)c3)c2)cc1]

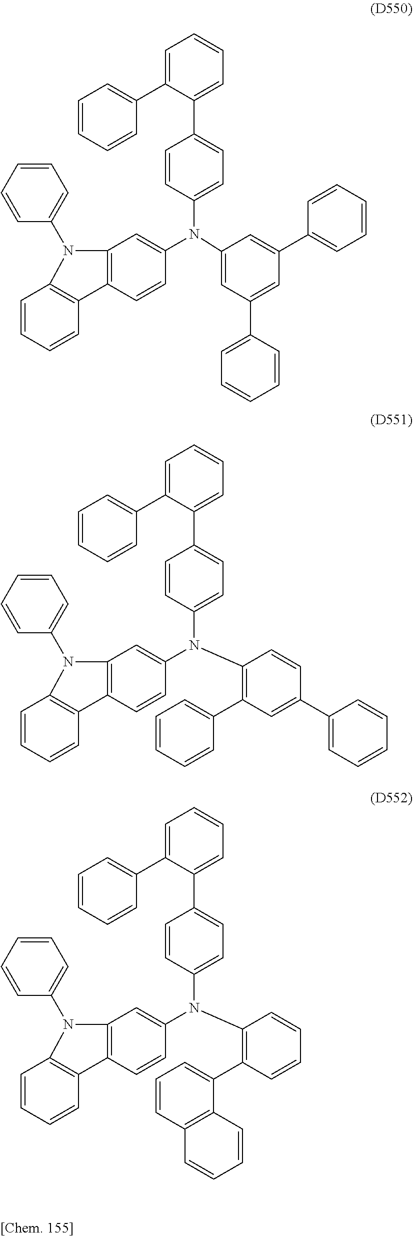

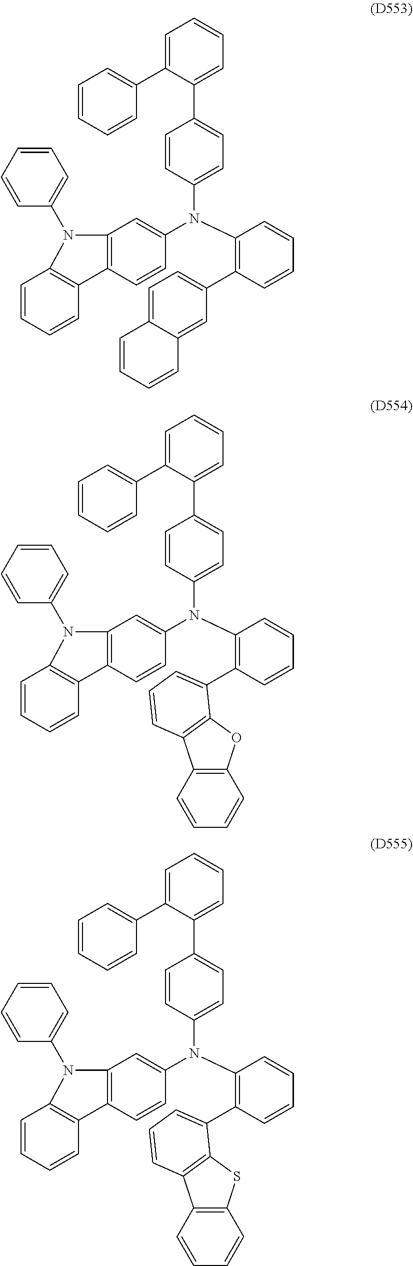

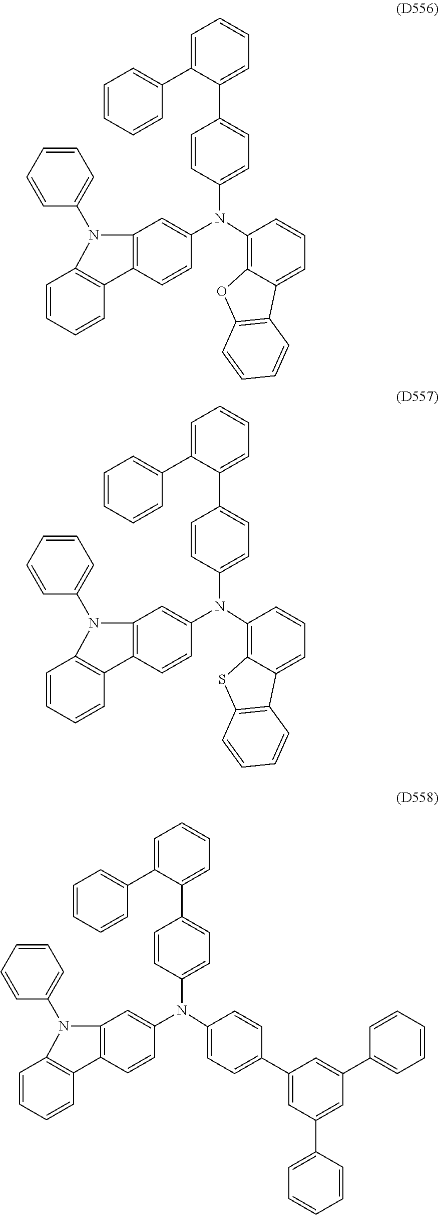

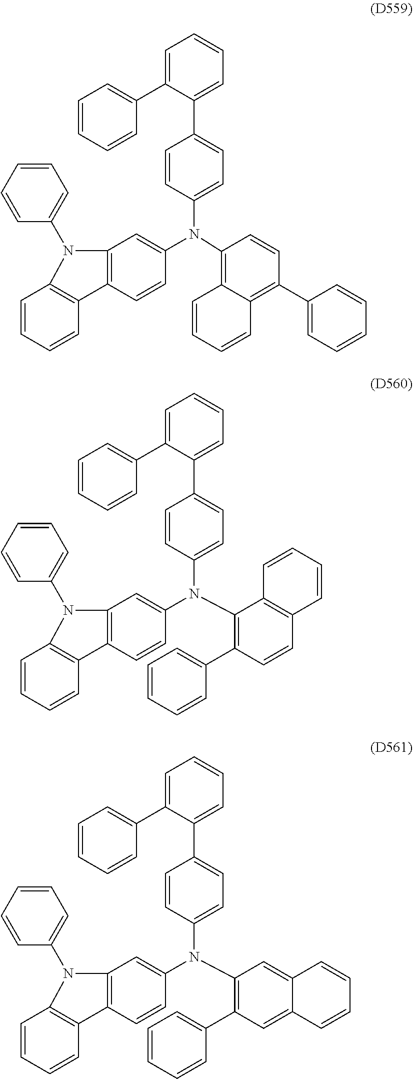

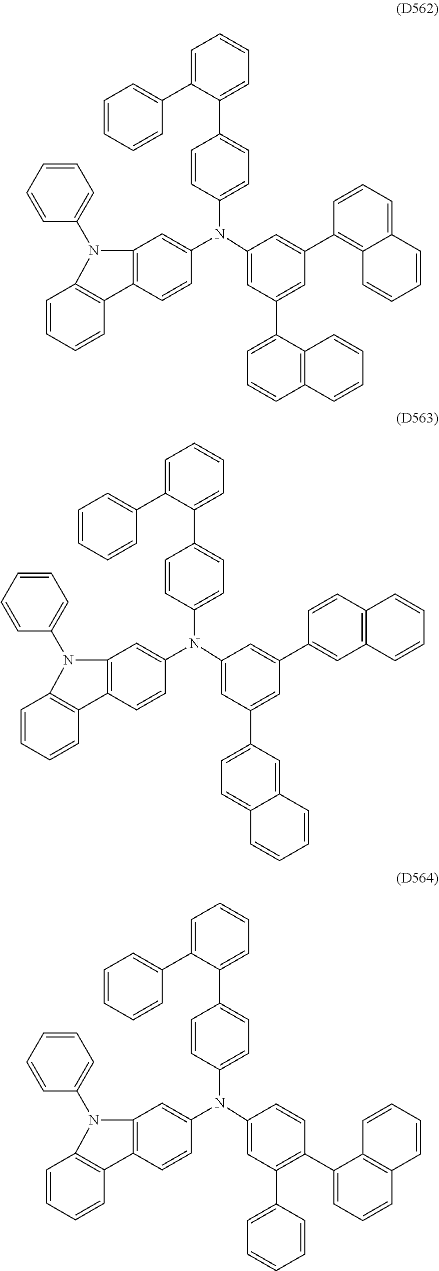 [SMILES: c1ccc(-c2ccccc2-c2ccc(N(c3cc(-c4cccc5ccccc45)cc(-c4cccc5ccccc45)c3)c3ccc4c5ccccc5n(-c5ccccc5)c4c3)cc2)cc1 ; c1ccc(-c2ccccc2-c2ccc(N(c3cc(-c4ccc5ccccc5c4)cc(-c4ccc5ccccc5c4)c3)c3ccc4c5ccccc5n(-c5ccccc5)c4c3)cc2)cc1 ; c1ccc(-c2ccccc2-c2ccc(N(c3ccc(-c4cccc5ccccc45)c(-c4ccccc4)c3)c3ccc4c5ccccc5n(-c5ccccc5)c4c3)cc2)cc1]

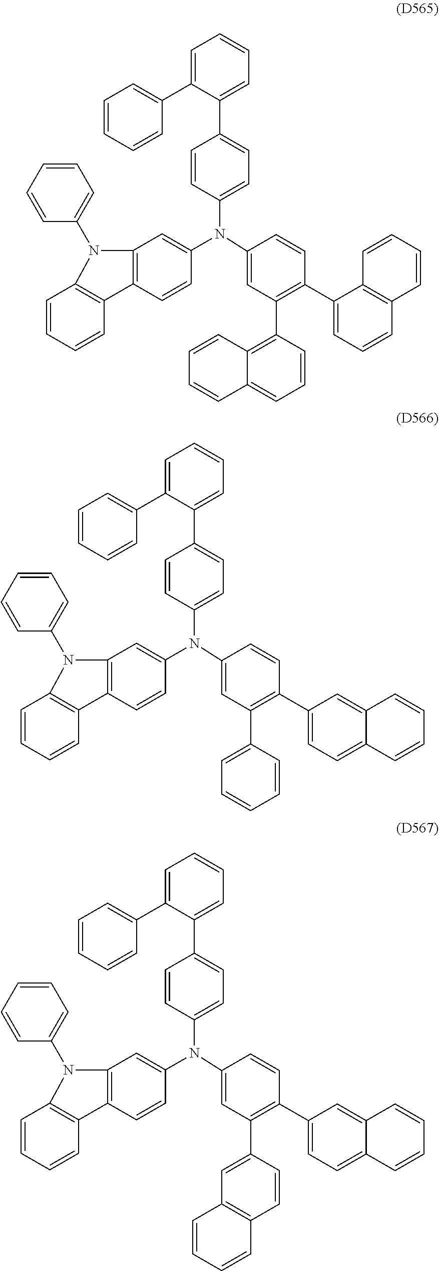 [SMILES: c1ccc(-c2ccccc2-c2ccc(N(c3ccc(-c4cccc5ccccc45)c(-c4cccc5ccccc45)c3)c3ccc4c5ccccc5n(-c5ccccc5)c4c3)cc2)cc1 ; c1ccc(-c2ccccc2-c2ccc(N(c3ccc(-c4ccc5ccccc5c4)c(-c4ccccc4)c3)c3ccc4c5ccccc5n(-c5ccccc5)c4c3)cc2)cc1 ; c1ccc(-c2ccccc2-c2ccc(N(c3ccc(-c4ccc5ccccc5c4)c(-c4ccc5ccccc5c4)c3)c3ccc4c5ccccc5n(-c5ccccc5)c4c3)cc2)cc1]

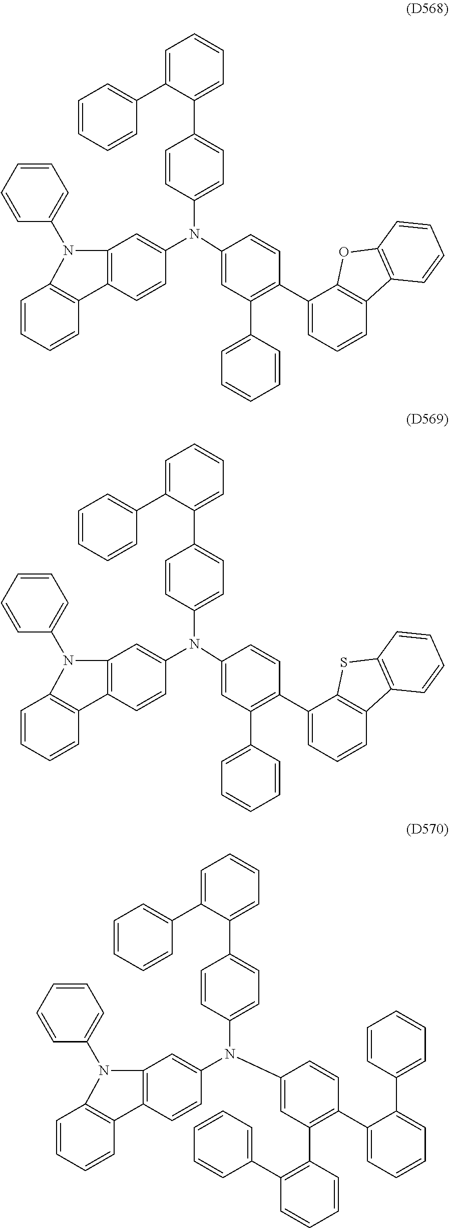 [SMILES: c1ccc(-c2ccccc2-c2ccc(N(c3ccc(-c4cccc5c4oc4ccccc45)c(-c4ccccc4)c3)c3ccc4c5ccccc5n(-c5ccccc5)c4c3)cc2)cc1 ; c1ccc(-c2ccccc2-c2ccc(N(c3ccc(-c4cccc5c4sc4ccccc45)c(-c4ccccc4)c3)c3ccc4c5ccccc5n(-c5ccccc5)c4c3)cc2)cc1 ; c1ccc(-c2ccccc2-c2ccc(N(c3ccc(-c4ccccc4-c4ccccc4)c(-c4ccccc4-c4ccccc4)c3)c3ccc4c5ccccc5n(-c5ccccc5)c4c3)cc2)cc1]

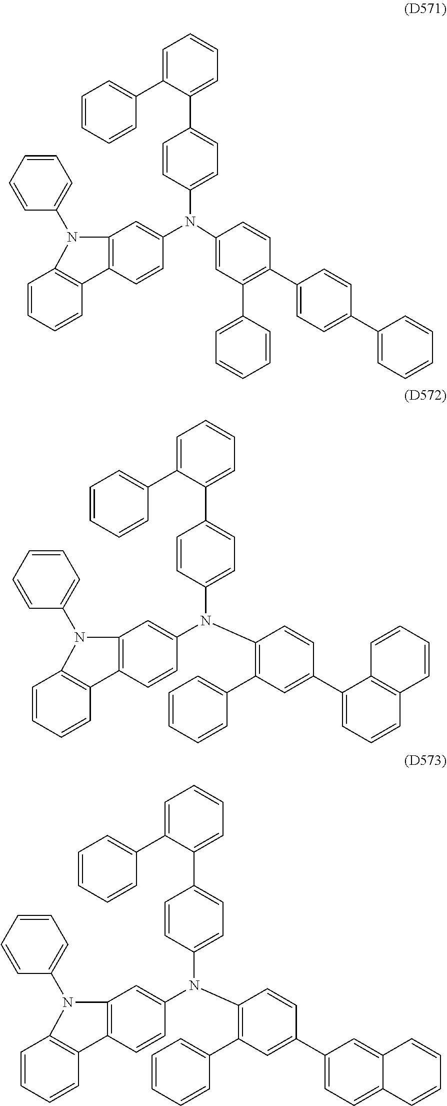

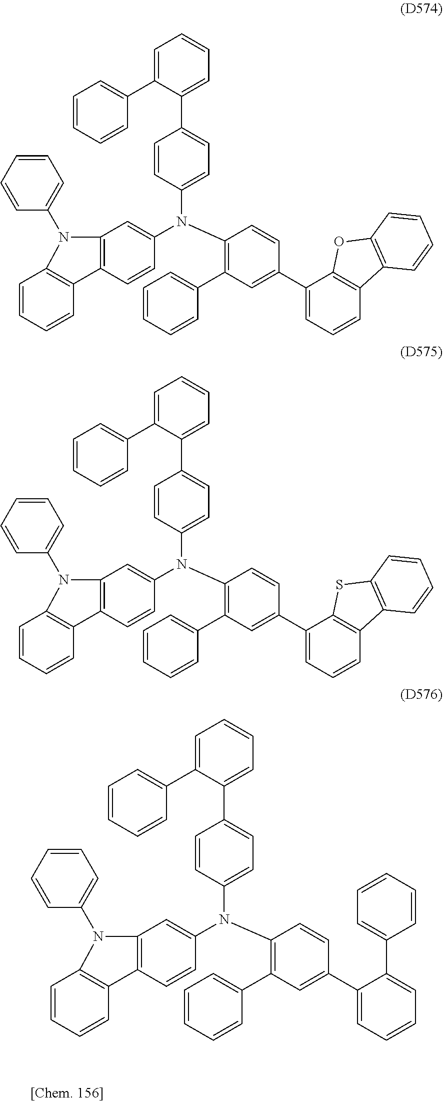

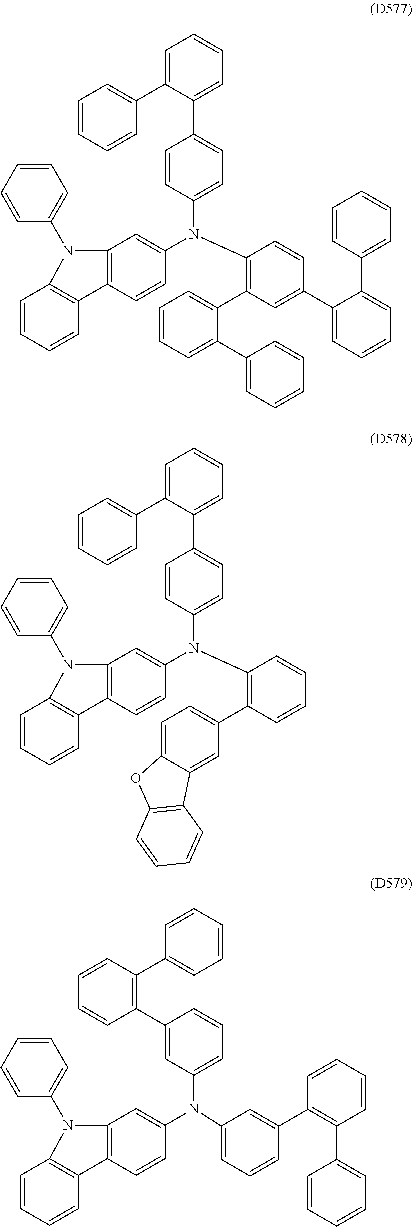

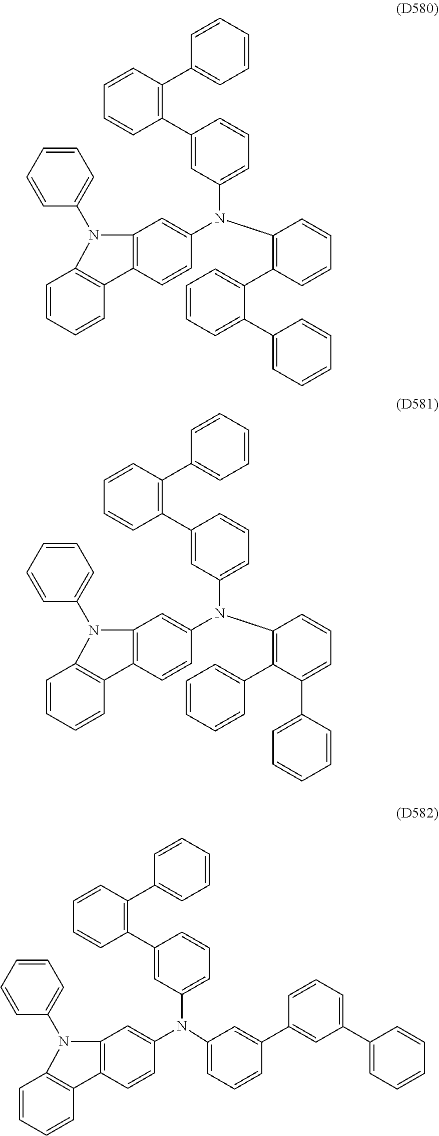 [SMILES: c1ccc(-c2ccccc2-c2cccc(N(c3ccc4c5ccccc5n(-c5ccccc5)c4c3)c3ccccc3-c3ccccc3-c3ccccc3)c2)cc1 ; c1ccc(-c2ccccc2-c2cccc(N(c3ccc4c5ccccc5n(-c5ccccc5)c4c3)c3cccc(-c4ccccc4)c3-c3ccccc3)c2)cc1 ; c1ccc(-c2cccc(-c3cccc(N(c4cccc(-c5ccccc5-c5ccccc5)c4)c4ccc5c6ccccc6n(-c6ccccc6)c5c4)c3)c2)cc1]

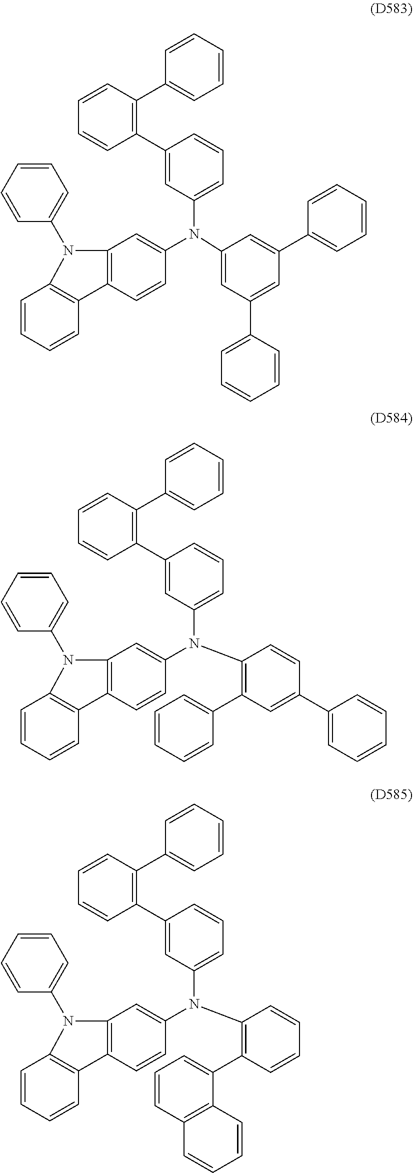

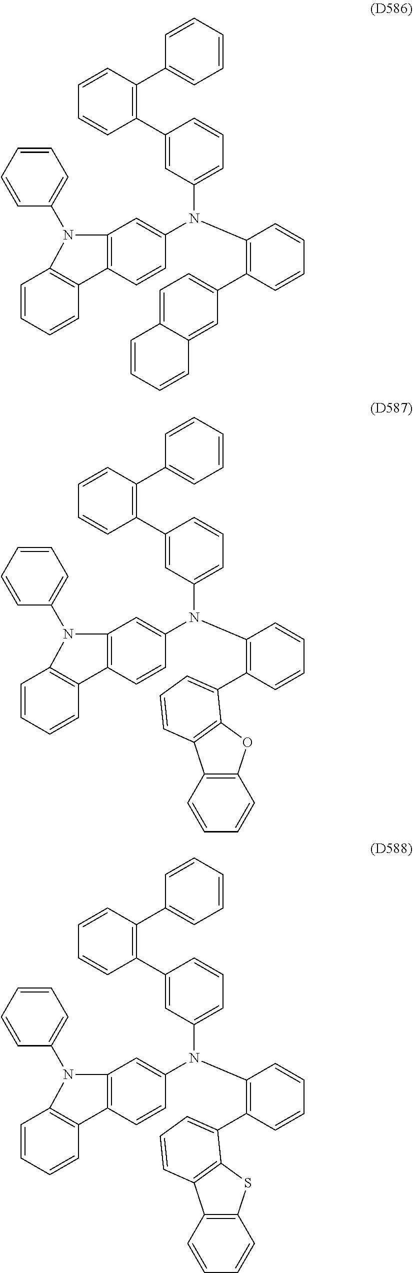

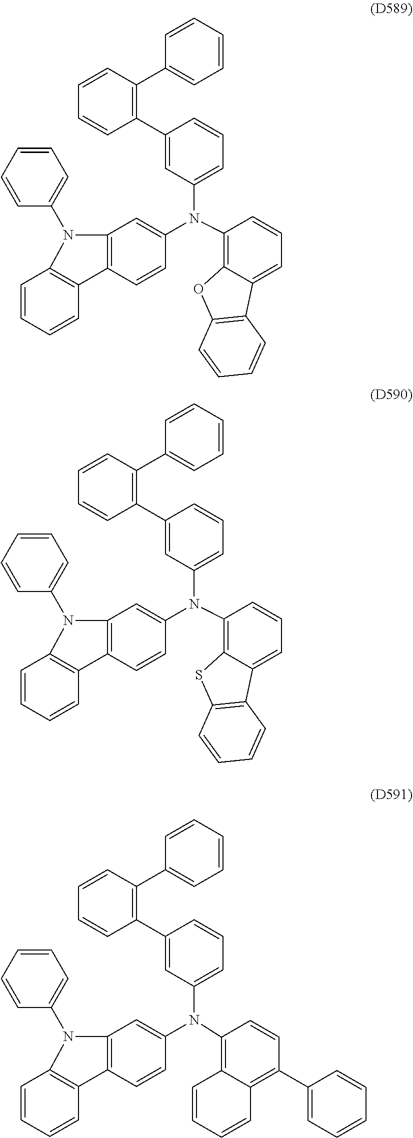

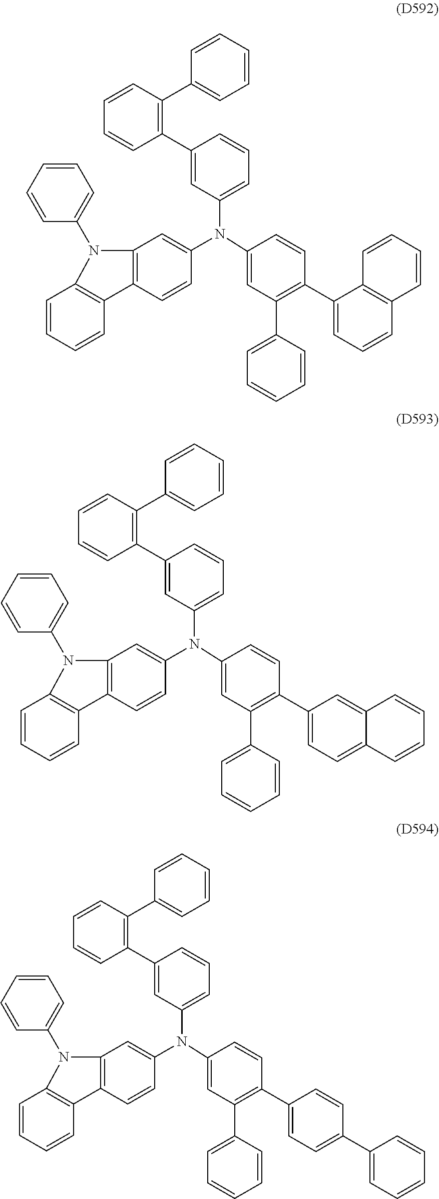 [SMILES: c1ccc(-c2ccccc2-c2cccc(N(c3ccc(-c4cccc5ccccc45)c(-c4ccccc4)c3)c3ccc4c5ccccc5n(-c5ccccc5)c4c3)c2)cc1 ; c1ccc(-c2ccccc2-c2cccc(N(c3ccc(-c4ccc5ccccc5c4)c(-c4ccccc4)c3)c3ccc4c5ccccc5n(-c5ccccc5)c4c3)c2)cc1 ; c1ccc(-c2ccc(-c3ccc(N(c4cccc(-c5ccccc5-c5ccccc5)c4)c4ccc5c6ccccc6n(-c6ccccc6)c5c4)cc3-c3ccccc3)cc2)cc1]

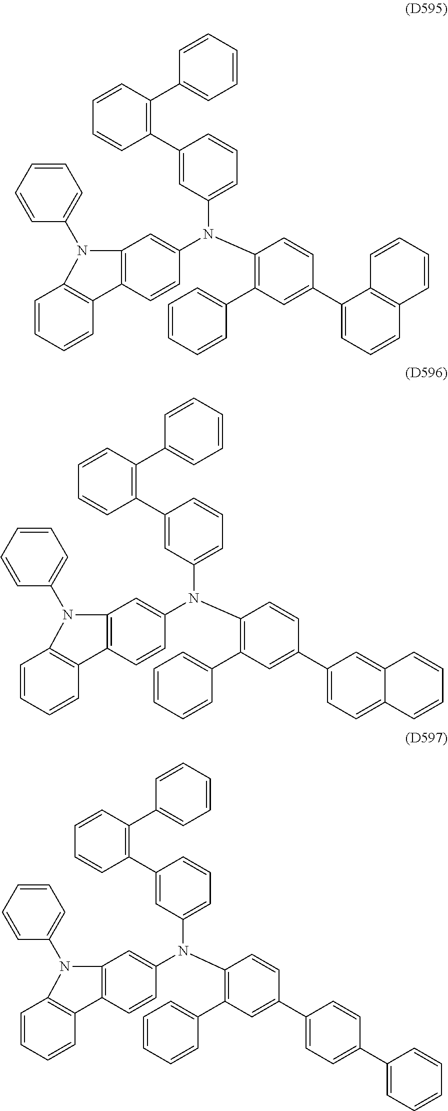

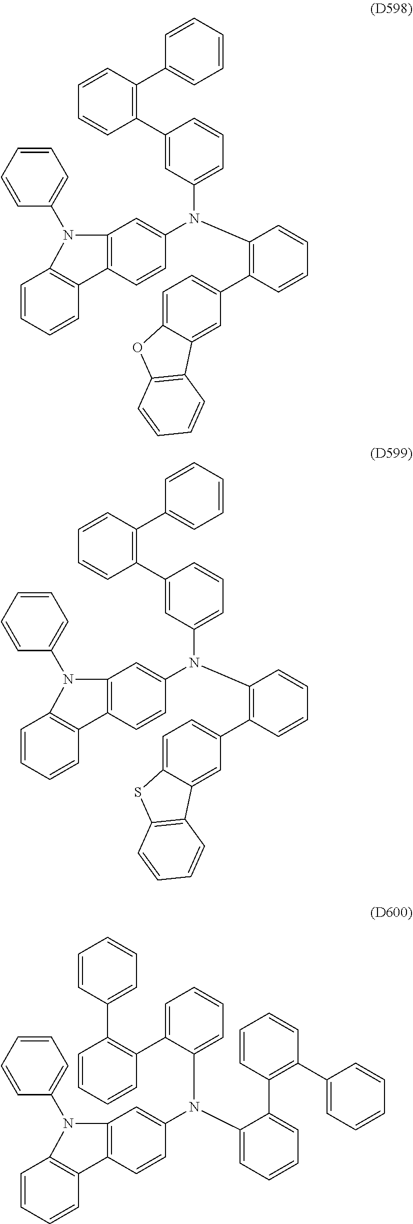

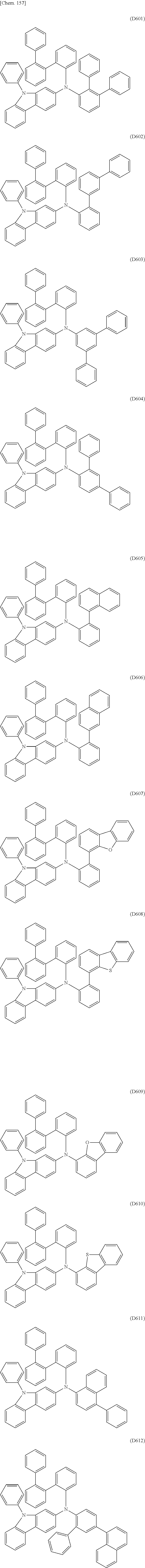

[chemical structure]

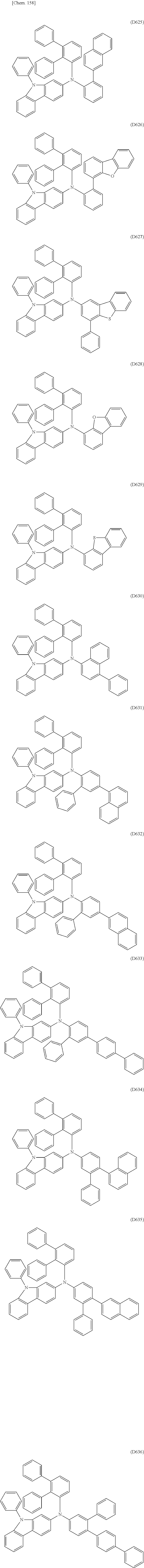

[chemical structure]

[chemical structure]

[chemical structure]

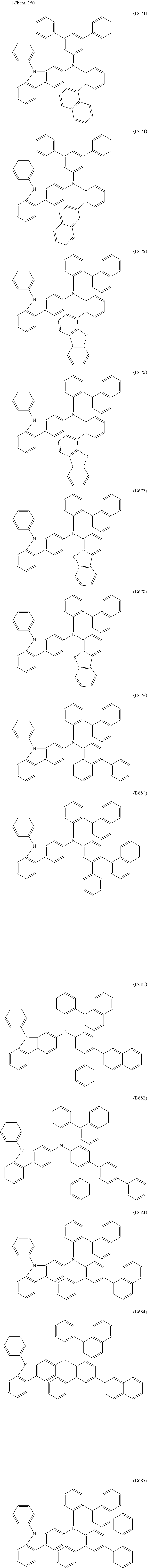

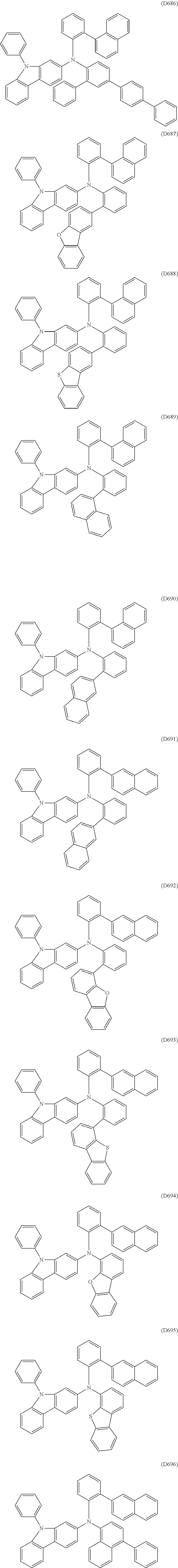

[chemical structure]

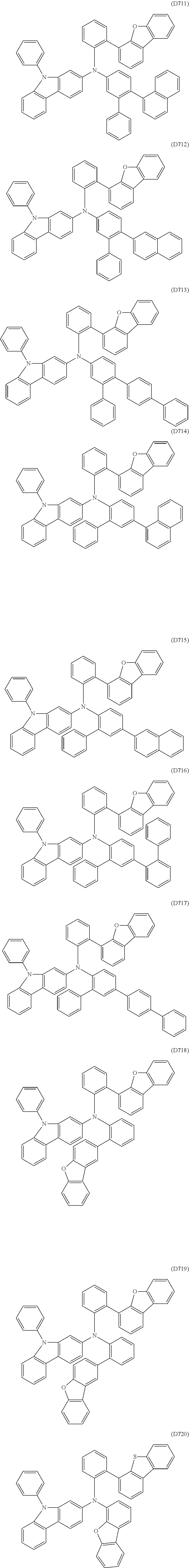

[chemical structure]

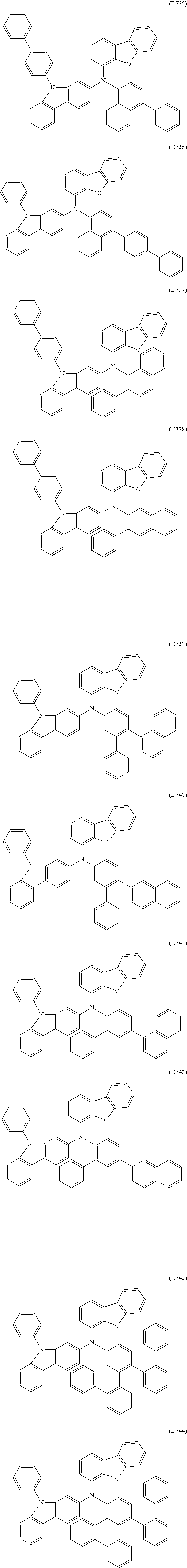

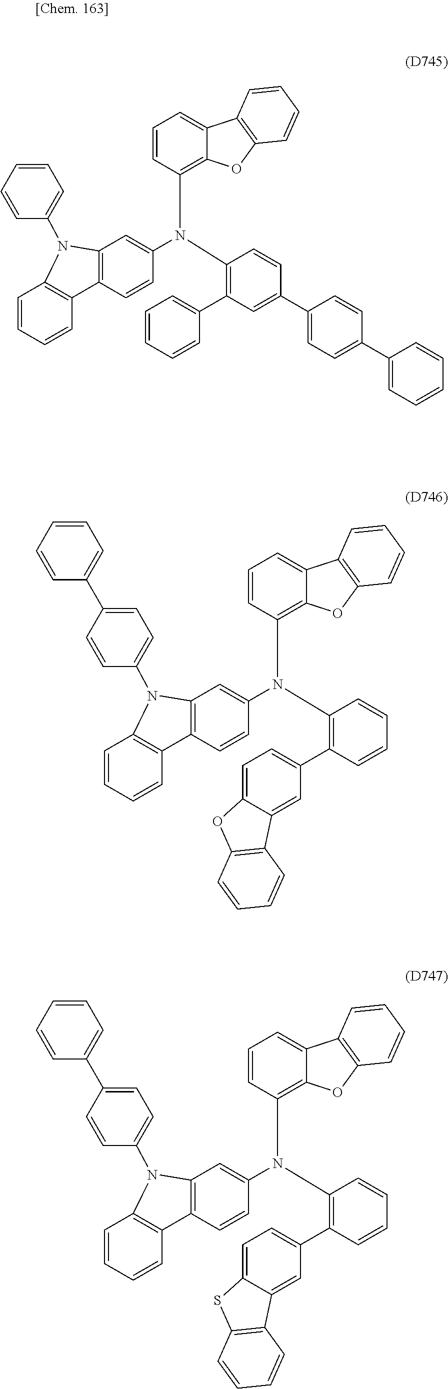 [SMILES: c1ccc(-c2ccc(-c3ccc(N(c4ccc5c6ccccc6n(-c6ccccc6)c5c4)c4cccc5c4oc4ccccc45)c(-c4ccccc4)c3)cc2)cc1 ; c1ccc(-c2ccc(-n3c4ccccc4c4ccc(N(c5ccccc5-c5ccc6oc7ccccc7c6c5)c5cccc6c5oc5ccccc56)cc43)cc2)cc1 ; c1ccc(-c2ccc(-n3c4ccccc4c4ccc(N(c5ccccc5-c5ccc6sc7ccccc7c6c5)c5cccc6c5oc5ccccc56)cc43)cc2)cc1]

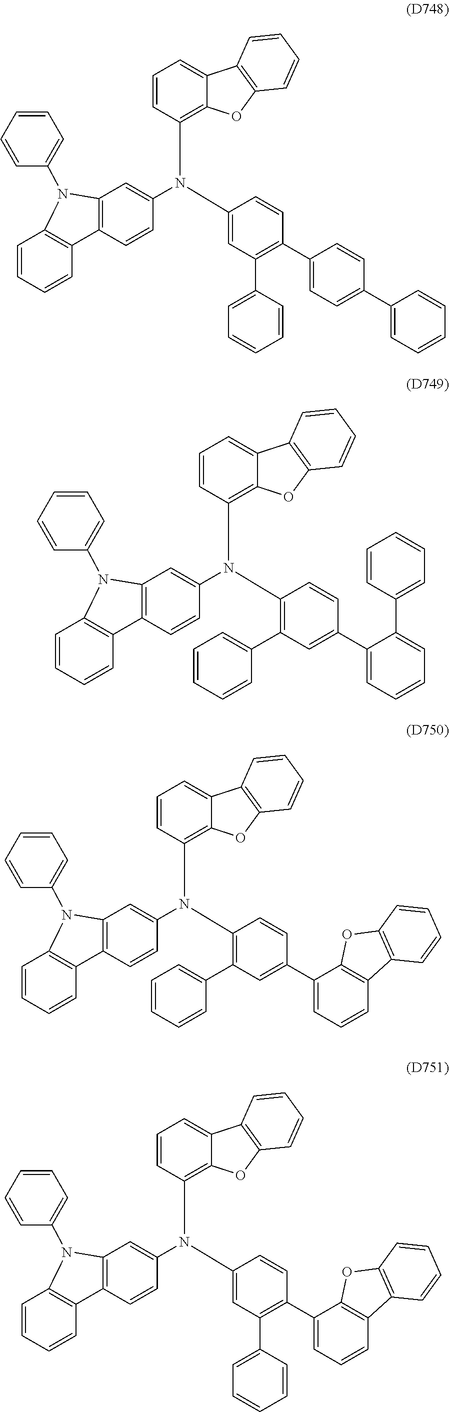 [SMILES: c1ccc(-c2ccc(-c3ccc(N(c4ccc5c6ccccc6n(-c6ccccc6)c5c4)c4cccc5c4oc4ccccc45)cc3-c3ccccc3)cc2)cc1 ; c1ccc(-c2ccccc2-c2ccc(N(c3ccc4c5ccccc5n(-c5ccccc5)c4c3)c3cccc4c3oc3ccccc34)c(-c3ccccc3)c2)cc1 ; c1ccc(-c2cc(-c3cccc4c3oc3ccccc34)ccc2N(c2ccc3c4ccccc4n(-c4ccccc4)c3c2)c2cccc3c2oc2ccccc23)cc1 ; c1ccc(-c2cc(N(c3ccc4c5ccccc5n(-c5ccccc5)c4c3)c3cccc4c3oc3ccccc34)ccc2-c2cccc3c2oc2ccccc23)cc1]

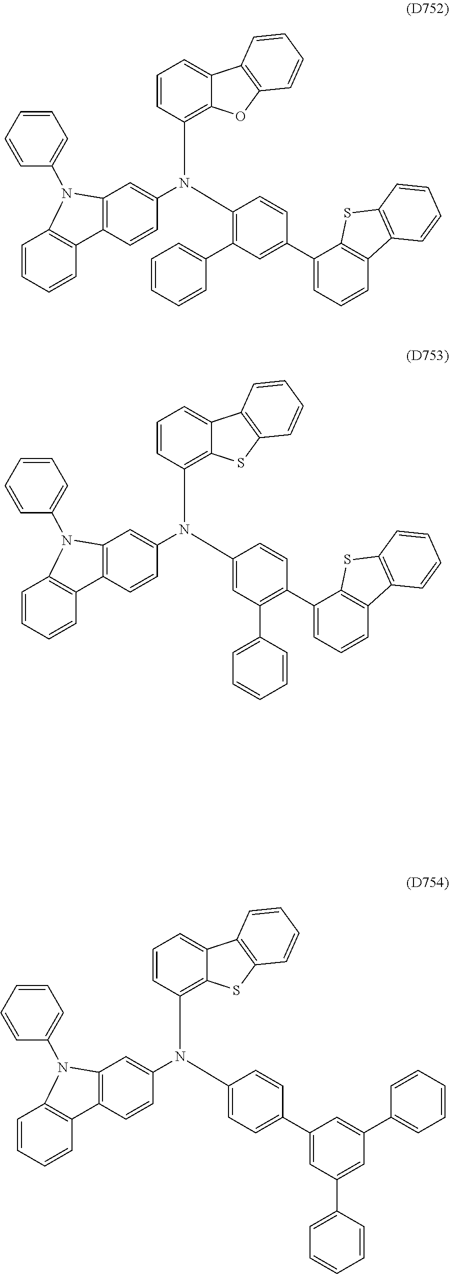 [SMILES: c1ccc(-c2cc(-c3cccc4c3sc3ccccc34)ccc2N(c2ccc3c4ccccc4n(-c4ccccc4)c3c2)c2cccc3c2oc2ccccc23)cc1 ; c1ccc(-c2cc(N(c3ccc4c5ccccc5n(-c5ccccc5)c4c3)c3cccc4c3sc3ccccc34)ccc2-c2cccc3c2sc2ccccc23)cc1 ; c1ccc(-c2cc(-c3ccccc3)cc(-c3ccc(N(c4ccc5c6ccccc6n(-c6ccccc6)c5c4)c4cccc5c4sc4ccccc45)cc3)c2)cc1]

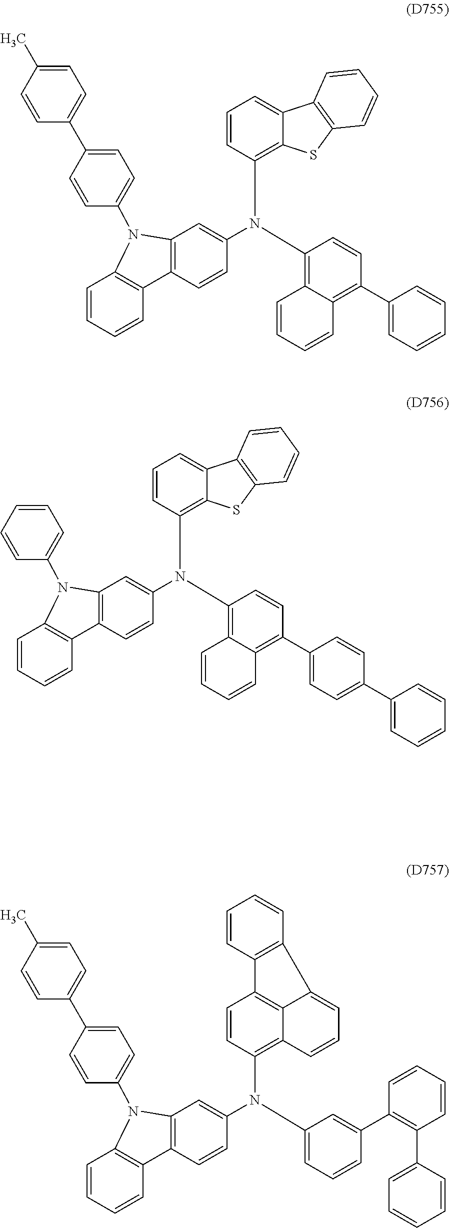 [SMILES: Cc1ccc(-c2ccc(-n3c4ccccc4c4ccc(N(c5ccc(-c6ccccc6)c6ccccc56)c5cccc6c5sc5ccccc56)cc43)cc2)cc1 ; c1ccc(-c2ccc(-c3ccc(N(c4ccc5c6ccccc6n(-c6ccccc6)c5c4)c4cccc5c4sc4ccccc45)c4ccccc34)cc2)cc1 ; Cc1ccc(-c2ccc(-n3c4ccccc4c4ccc(N(c5cccc(-c6ccccc6-c6ccccc6)c5)c5ccc6c7c(cccc57)-c5ccccc5-6)cc43)cc2)cc1]

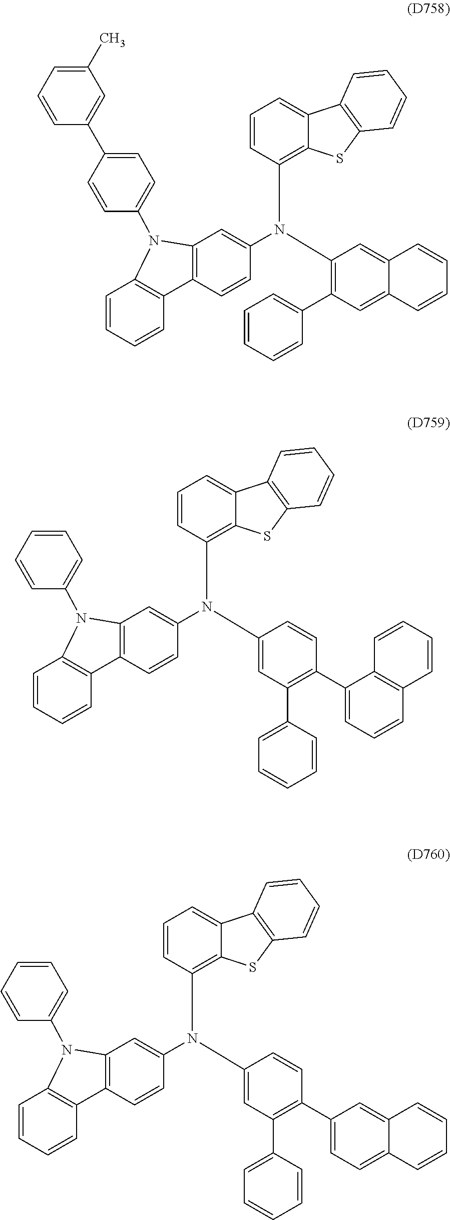 [SMILES: Cc1cccc(-c2ccc(-n3c4ccccc4c4ccc(N(c5cc6ccccc6cc5-c5ccccc5)c5cccc6c5sc5ccccc56)cc43)cc2)c1 ; c1ccc(-c2cc(N(c3ccc4c5ccccc5n(-c5ccccc5)c4c3)c3cccc4c3sc3ccccc34)ccc2-c2cccc3ccccc23)cc1 ; c1ccc(-c2cc(N(c3ccc4c5ccccc5n(-c5ccccc5)c4c3)c3cccc4c3sc3ccccc34)ccc2-c2ccc3ccccc3c2)cc1]

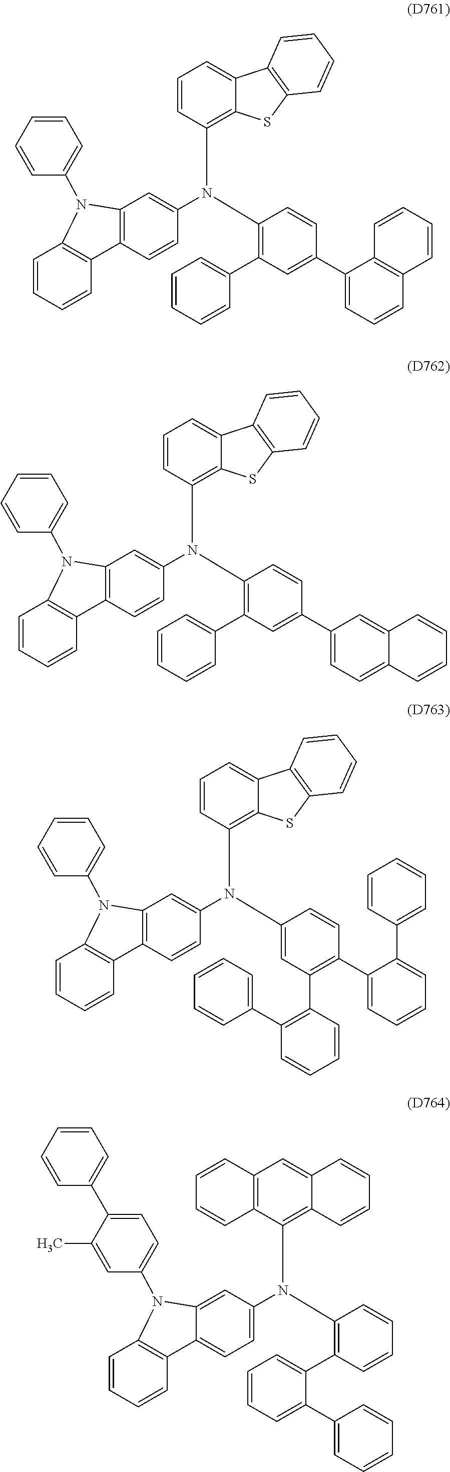 [SMILES: c1ccc(-c2cc(-c3cccc4ccccc34)ccc2N(c2ccc3c4ccccc4n(-c4ccccc4)c3c2)c2cccc3c2sc2ccccc23)cc1 ; c1ccc(-c2cc(-c3ccc4ccccc4c3)ccc2N(c2ccc3c4ccccc4n(-c4ccccc4)c3c2)c2cccc3c2sc2ccccc23)cc1 ; c1ccc(-c2ccccc2-c2ccc(N(c3ccc4c5ccccc5n(-c5ccccc5)c4c3)c3cccc4c3sc3ccccc34)cc2-c2ccccc2-c2ccccc2)cc1 ; Cc1cc(-n2c3ccccc3c3ccc(N(c4ccccc4-c4ccccc4-c4ccccc4)c4c5ccccc5cc5ccccc45)cc32)ccc1-c1ccccc1]

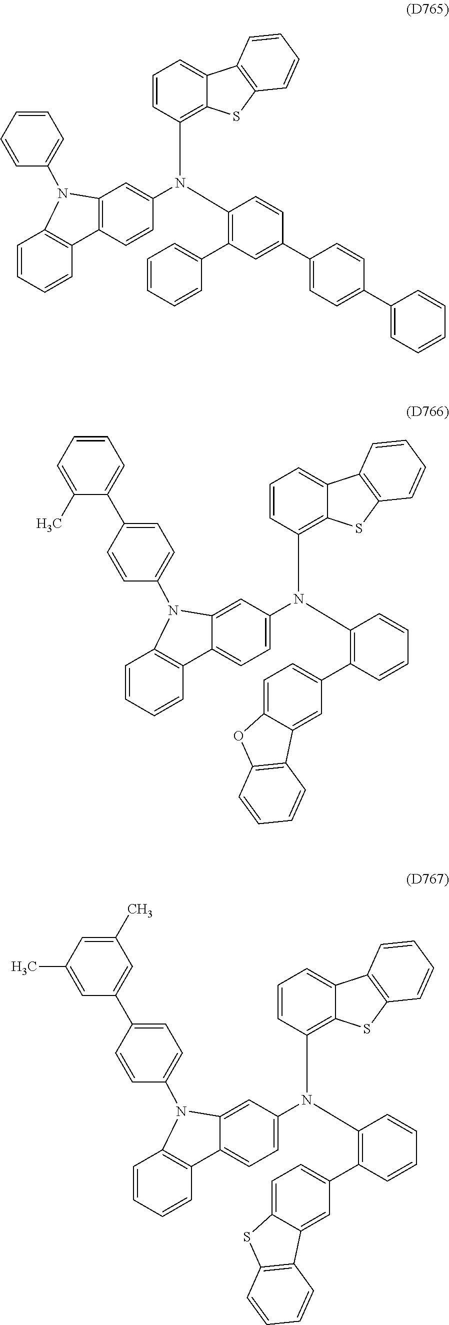 [SMILES: c1ccc(-c2ccc(-c3ccc(N(c4ccc5c6ccccc6n(-c6ccccc6)c5c4)c4cccc5c4sc4ccccc45)c(-c4ccccc4)c3)cc2)cc1 ; Cc1ccccc1-c1ccc(-n2c3ccccc3c3ccc(N(c4ccccc4-c4ccc5oc6ccccc6c5c4)c4cccc5c4sc4ccccc45)cc32)cc1 ; Cc1cc(C)cc(-c2ccc(-n3c4ccccc4c4ccc(N(c5ccccc5-c5ccc6sc7ccccc7c6c5)c5cccc6c5sc5ccccc56)cc43)cc2)c1]

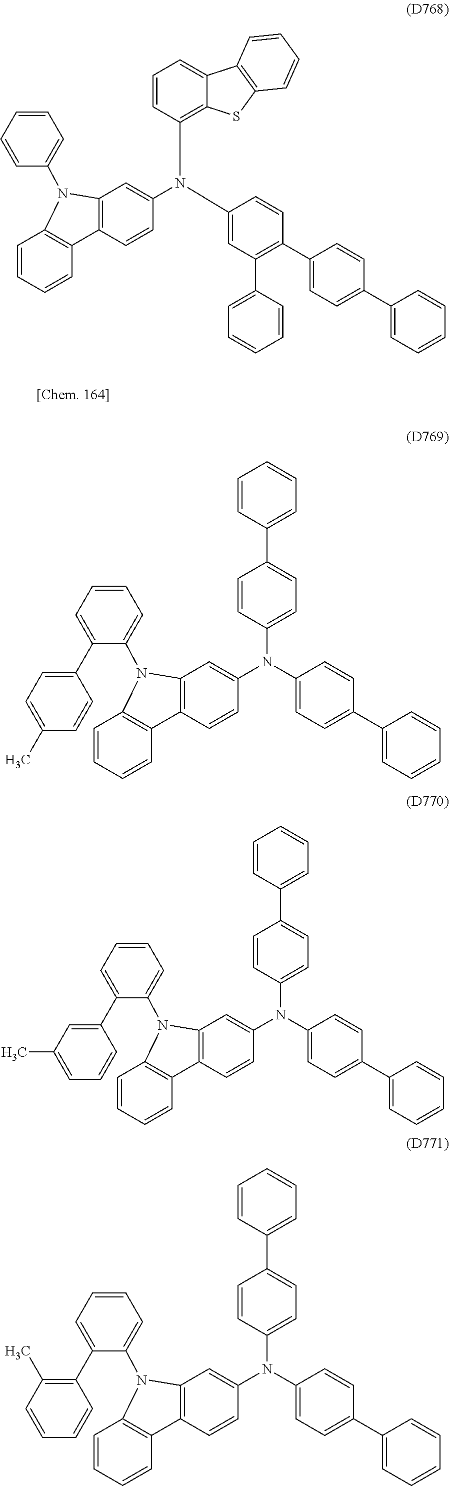 [SMILES: c1ccc(-c2ccc(-c3ccc(N(c4ccc5c6ccccc6n(-c6ccccc6)c5c4)c4cccc5c4sc4ccccc45)cc3-c3ccccc3)cc2)cc1 ; Cc1ccc(-c2ccccc2-n2c3ccccc3c3ccc(N(c4ccc(-c5ccccc5)cc4)c4ccc(-c5ccccc5)cc4)cc32)cc1 ; Cc1cccc(-c2ccccc2-n2c3ccccc3c3ccc(N(c4ccc(-c5ccccc5)cc4)c4ccc(-c5ccccc5)cc4)cc32)c1 ; Cc1ccccc1-c1ccccc1-n1c2ccccc2c2ccc(N(c3ccc(-c4ccccc4)cc3)c3ccc(-c4ccccc4)cc3)cc21]

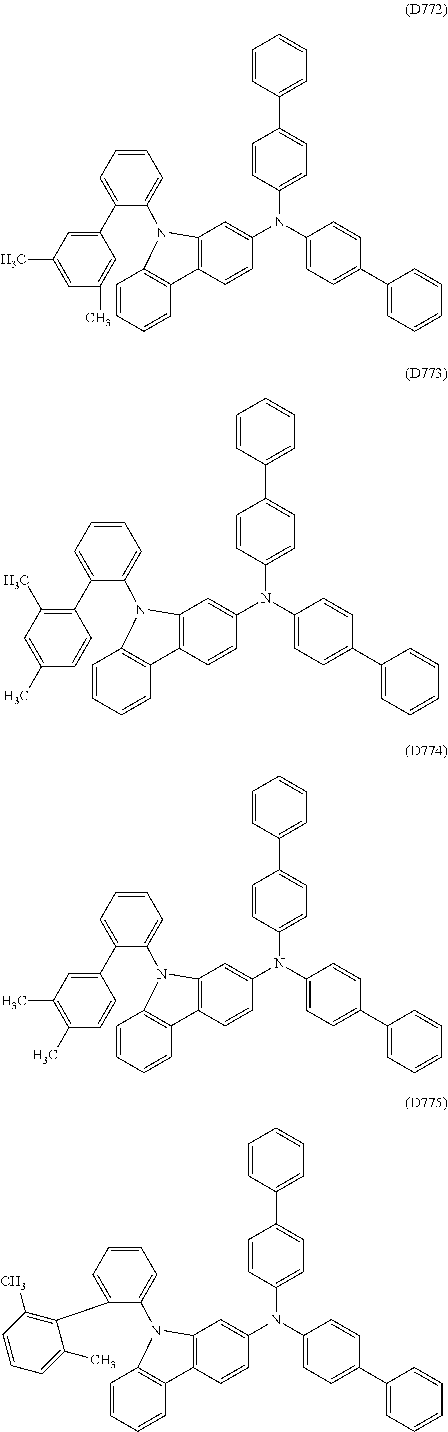 [SMILES: Cc1cc(C)cc(-c2ccccc2-n2c3ccccc3c3ccc(N(c4ccc(-c5ccccc5)cc4)c4ccc(-c5ccccc5)cc4)cc32)c1 ; Cc1ccc(-c2ccccc2-n2c3ccccc3c3ccc(N(c4ccc(-c5ccccc5)cc4)c4ccc(-c5ccccc5)cc4)cc32)c(C)c1 ; Cc1ccc(-c2ccccc2-n2c3ccccc3c3ccc(N(c4ccc(-c5ccccc5)cc4)c4ccc(-c5ccccc5)cc4)cc32)cc1C ; Cc1cccc(C)c1-c1ccccc1-n1c2ccccc2c2ccc(N(c3ccc(-c4ccccc4)cc3)c3ccc(-c4ccccc4)cc3)cc21]

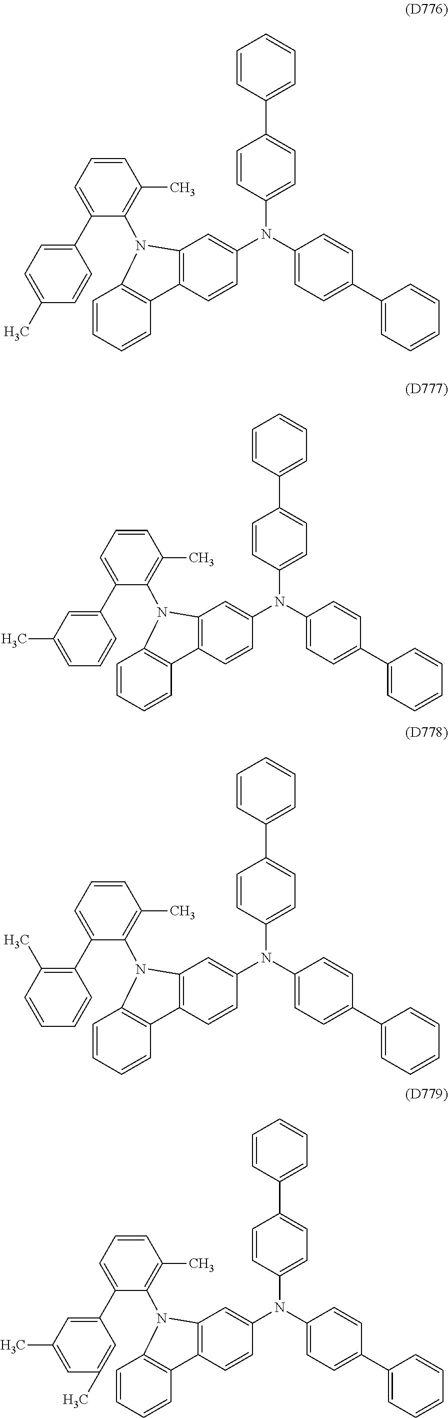 [SMILES: Cc1ccc(-c2cccc(C)c2-n2c3ccccc3c3ccc(N(c4ccc(-c5ccccc5)cc4)c4ccc(-c5ccccc5)cc4)cc32)cc1 ; Cc1cccc(-c2cccc(C)c2-n2c3ccccc3c3ccc(N(c4ccc(-c5ccccc5)cc4)c4ccc(-c5ccccc5)cc4)cc32)c1 ; Cc1ccccc1-c1cccc(C)c1-n1c2ccccc2c2ccc(N(c3ccc(-c4ccccc4)cc3)c3ccc(-c4ccccc4)cc3)cc21 ; Cc1cc(C)cc(-c2cccc(C)c2-n2c3ccccc3c3ccc(N(c4ccc(-c5ccccc5)cc4)c4ccc(-c5ccccc5)cc4)cc32)c1]

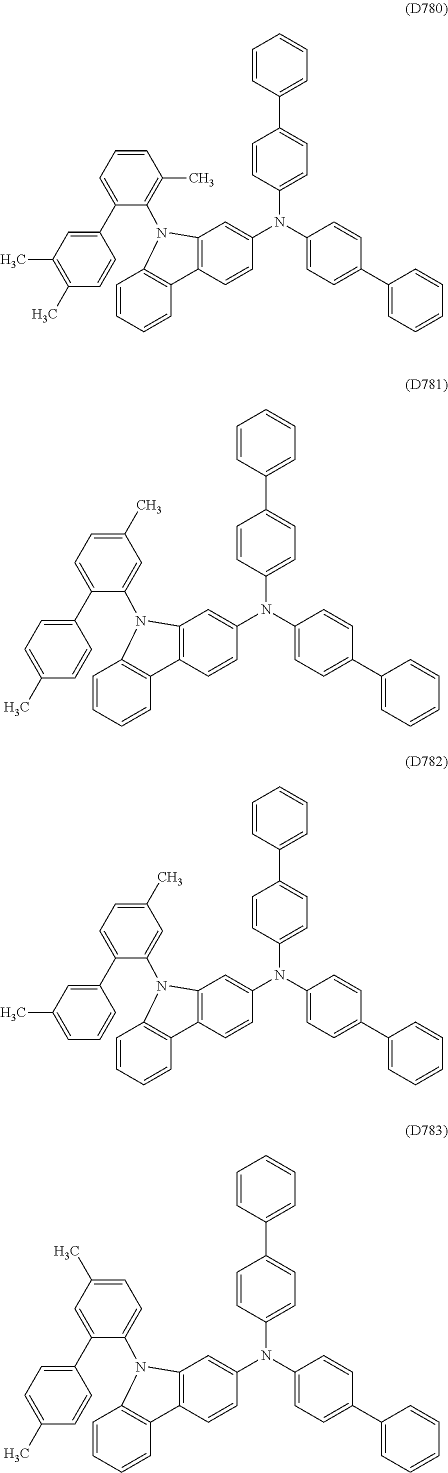 [SMILES: Cc1ccc(-c2cccc(C)c2-n2c3ccccc3c3ccc(N(c4ccc(-c5ccccc5)cc4)c4ccc(-c5ccccc5)cc4)cc32)cc1C ; Cc1ccc(-c2ccc(C)cc2-n2c3ccccc3c3ccc(N(c4ccc(-c5ccccc5)cc4)c4ccc(-c5ccccc5)cc4)cc32)cc1 ; Cc1cccc(-c2ccc(C)cc2-n2c3ccccc3c3ccc(N(c4ccc(-c5ccccc5)cc4)c4ccc(-c5ccccc5)cc4)cc32)c1 ; Cc1ccc(-c2cc(C)ccc2-n2c3ccccc3c3ccc(N(c4ccc(-c5ccccc5)cc4)c4ccc(-c5ccccc5)cc4)cc32)cc1]

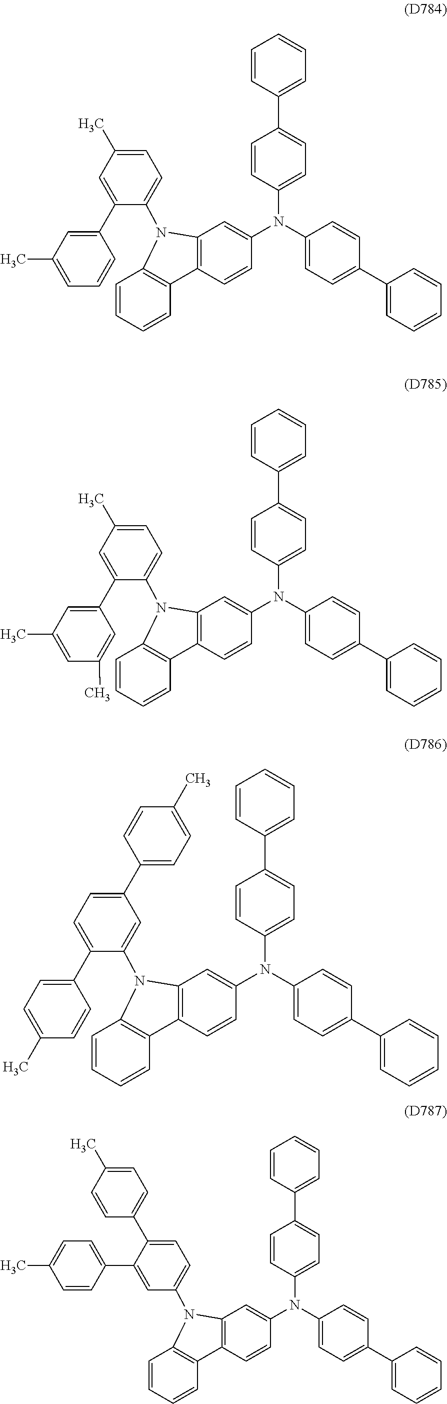 [SMILES: Cc1cccc(-c2cc(C)ccc2-n2c3ccccc3c3ccc(N(c4ccc(-c5ccccc5)cc4)c4ccc(-c5ccccc5)cc4)cc32)c1 ; Cc1cc(C)cc(-c2cc(C)ccc2-n2c3ccccc3c3ccc(N(c4ccc(-c5ccccc5)cc4)c4ccc(-c5ccccc5)cc4)cc32)c1 ; Cc1ccc(-c2ccc(-c3ccc(C)cc3)c(-n3c4ccccc4c4ccc(N(c5ccc(-c6ccccc6)cc5)c5ccc(-c6ccccc6)cc5)cc43)c2)cc1 ; Cc1ccc(-c2ccc(-n3c4ccccc4c4ccc(N(c5ccc(-c6ccccc6)cc5)c5ccc(-c6ccccc6)cc5)cc43)cc2-c2ccc(C)cc2)cc1]

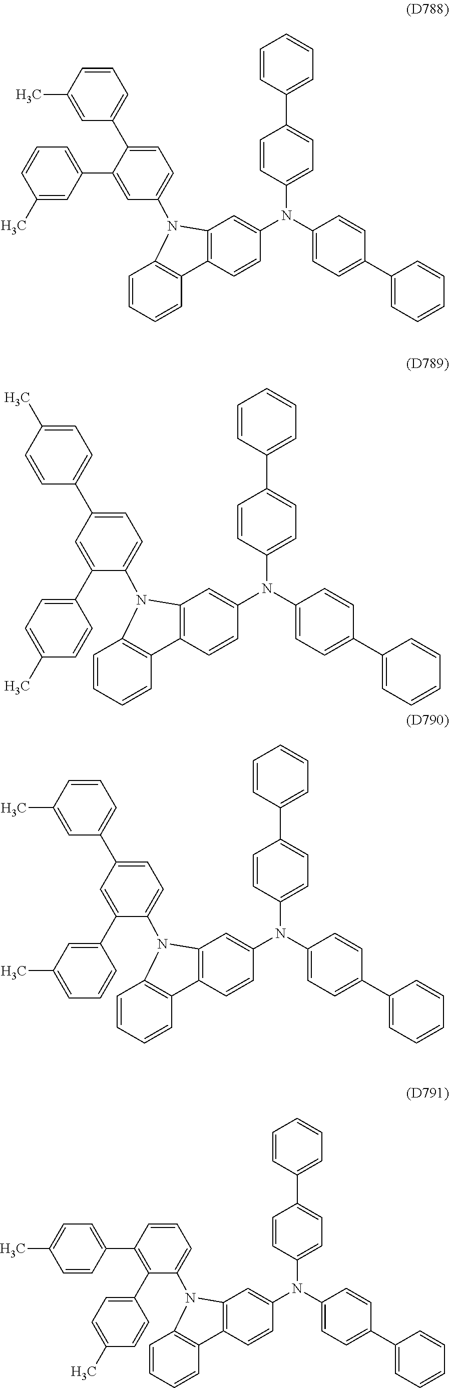 [SMILES: Cc1cccc(-c2ccc(-n3c4ccccc4c4ccc(N(c5ccc(-c6ccccc6)cc5)c5ccc(-c6ccccc6)cc5)cc43)cc2-c2cccc(C)c2)c1 ; Cc1ccc(-c2ccc(-n3c4ccccc4c4ccc(N(c5ccc(-c6ccccc6)cc5)c5ccc(-c6ccccc6)cc5)cc43)c(-c3ccc(C)cc3)c2)cc1 ; Cc1cccc(-c2ccc(-n3c4ccccc4c4ccc(N(c5ccc(-c6ccccc6)cc5)c5ccc(-c6ccccc6)cc5)cc43)c(-c3cccc(C)c3)c2)c1 ; Cc1ccc(-c2cccc(-n3c4ccccc4c4ccc(N(c5ccc(-c6ccccc6)cc5)c5ccc(-c6ccccc6)cc5)cc43)c2-c2ccc(C)cc2)cc1]

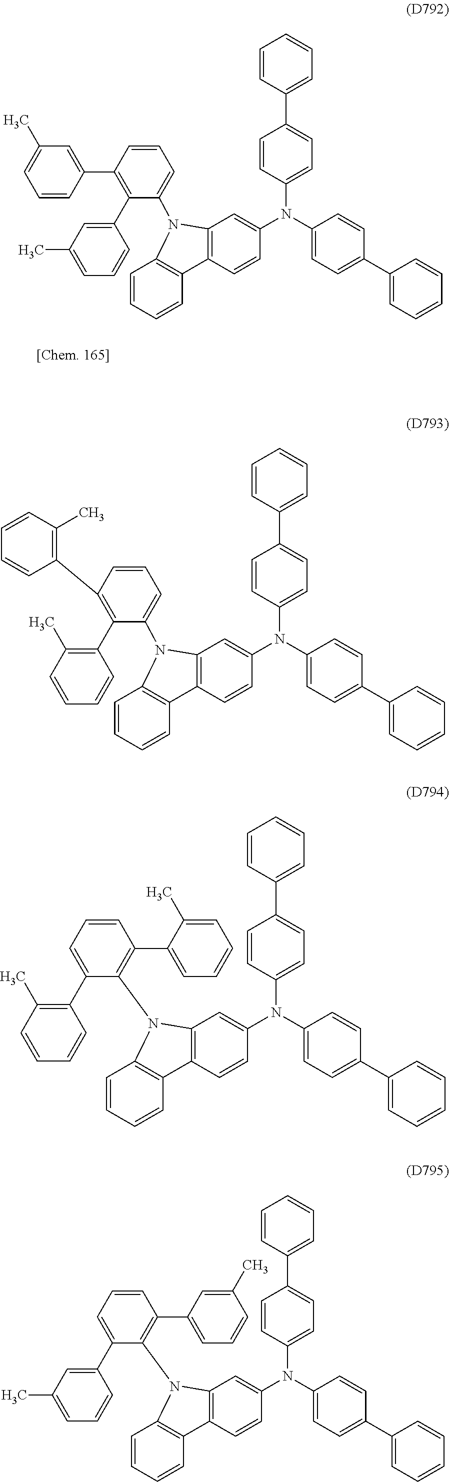 [SMILES: Cc1cccc(-c2cccc(-n3c4ccccc4c4ccc(N(c5ccc(-c6ccccc6)cc5)c5ccc(-c6ccccc6)cc5)cc43)c2-c2cccc(C)c2)c1 ; Cc1ccccc1-c1cccc(-n2c3ccccc3c3ccc(N(c4ccc(-c5ccccc5)cc4)c4ccc(-c5ccccc5)cc4)cc32)c1-c1ccccc1C ; Cc1ccccc1-c1cccc(-c2ccccc2C)c1-n1c2ccccc2c2ccc(N(c3ccc(-c4ccccc4)cc3)c3ccc(-c4ccccc4)cc3)cc21 ; Cc1cccc(-c2cccc(-c3cccc(C)c3)c2-n2c3ccccc3c3ccc(N(c4ccc(-c5ccccc5)cc4)c4ccc(-c5ccccc5)cc4)cc32)c1]

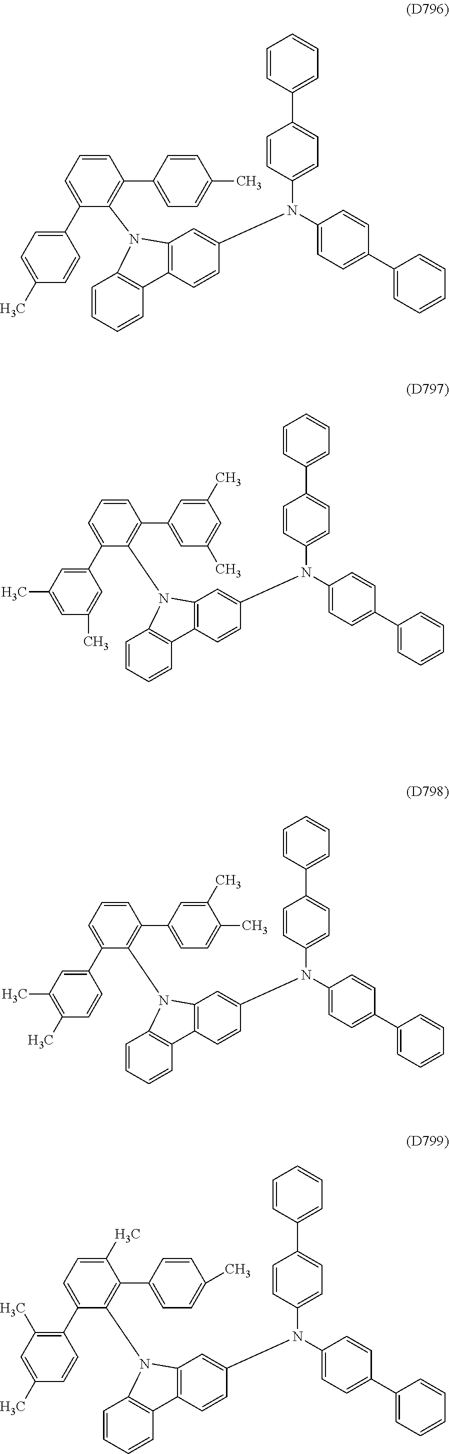 [SMILES: Cc1ccc(-c2cccc(-c3ccc(C)cc3)c2-n2c3ccccc3c3ccc(N(c4ccc(-c5ccccc5)cc4)c4ccc(-c5ccccc5)cc4)cc32)cc1 ; Cc1cc(C)cc(-c2cccc(-c3cc(C)cc(C)c3)c2-n2c3ccccc3c3ccc(N(c4ccc(-c5ccccc5)cc4)c4ccc(-c5ccccc5)cc4)cc32)c1 ; Cc1ccc(-c2cccc(-c3ccc(C)c(C)c3)c2-n2c3ccccc3c3ccc(N(c4ccc(-c5ccccc5)cc4)c4ccc(-c5ccccc5)cc4)cc32)cc1C ; Cc1ccc(-c2c(C)ccc(-c3ccc(C)cc3C)c2-n2c3ccccc3c3ccc(N(c4ccc(-c5ccccc5)cc4)c4ccc(-c5ccccc5)cc4)cc32)cc1]

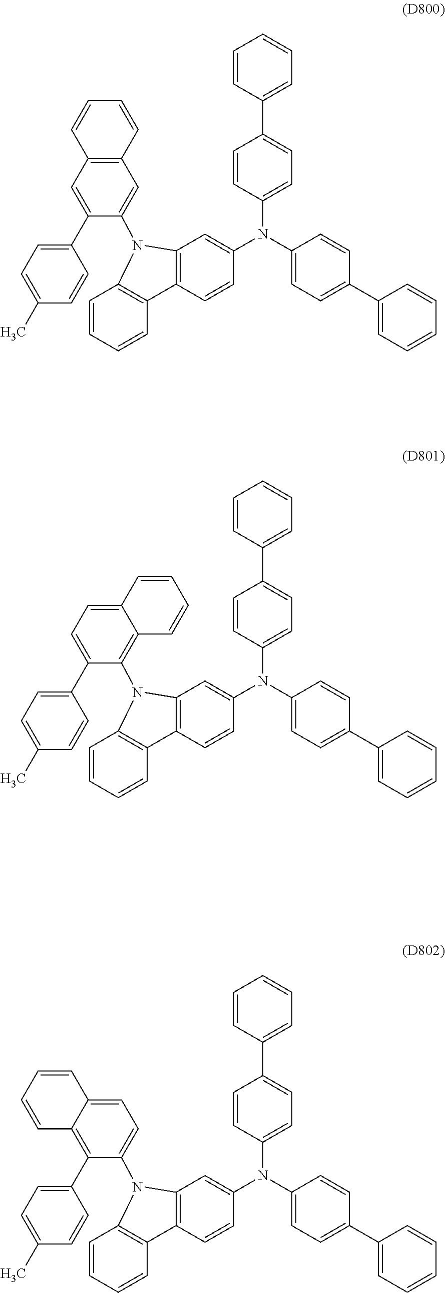 [SMILES: Cc1ccc(-c2cc3ccccc3cc2-n2c3ccccc3c3ccc(N(c4ccc(-c5ccccc5)cc4)c4ccc(-c5ccccc5)cc4)cc32)cc1 ; Cc1ccc(-c2ccc3ccccc3c2-n2c3ccccc3c3ccc(N(c4ccc(-c5ccccc5)cc4)c4ccc(-c5ccccc5)cc4)cc32)cc1 ; Cc1ccc(-c2c(-n3c4ccccc4c4ccc(N(c5ccc(-c6ccccc6)cc5)c5ccc(-c6ccccc6)cc5)cc43)ccc3ccccc23)cc1]

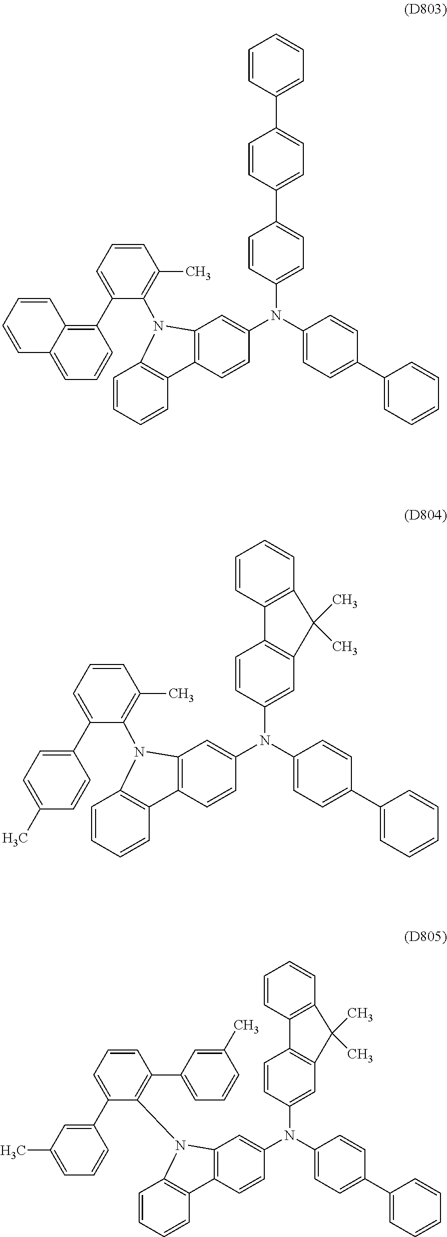 [SMILES: Cc1cccc(-c2cccc3ccccc23)c1-n1c2ccccc2c2ccc(N(c3ccc(-c4ccccc4)cc3)c3ccc(-c4ccc(-c5ccccc5)cc4)cc3)cc21 ; Cc1ccc(-c2cccc(C)c2-n2c3ccccc3c3ccc(N(c4ccc(-c5ccccc5)cc4)c4ccc5c(c4)C(C)(C)c4ccccc4-5)cc32)cc1 ; Cc1cccc(-c2cccc(-c3cccc(C)c3)c2-n2c3ccccc3c3ccc(N(c4ccc(-c5ccccc5)cc4)c4ccc5c(c4)C(C)(C)c4ccccc4-5)cc32)c1]

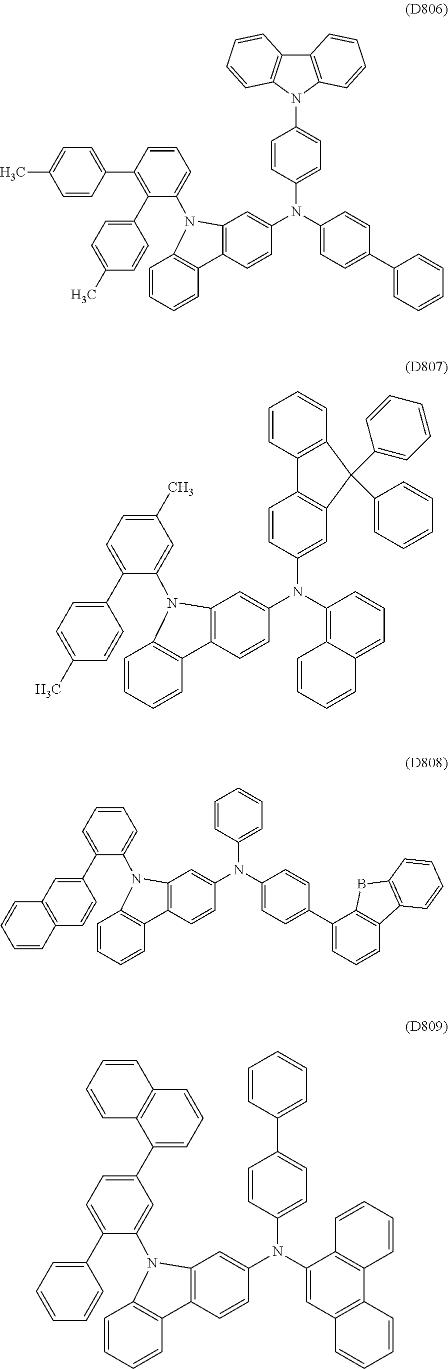

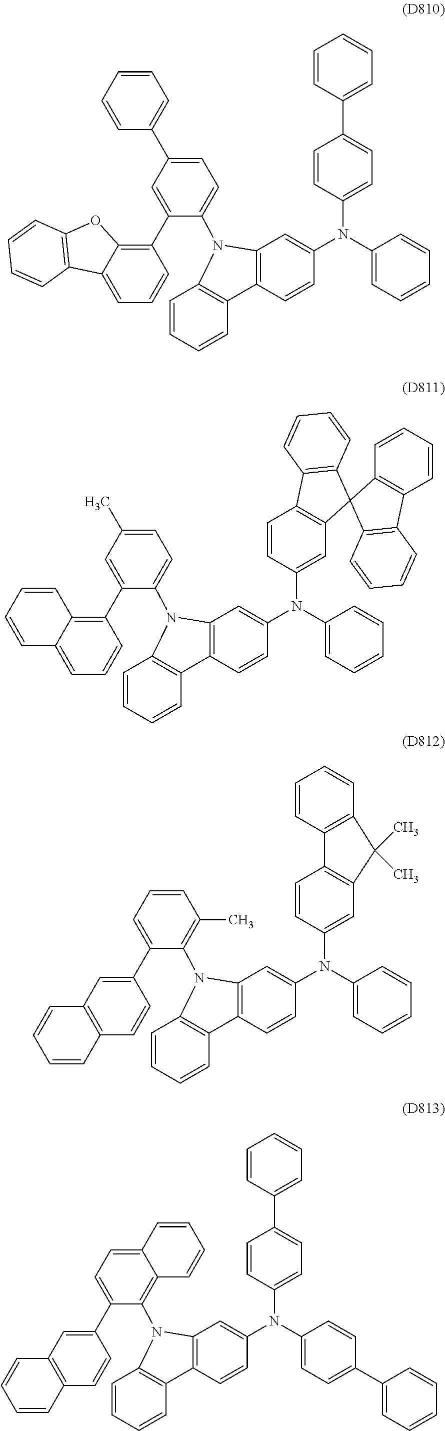 [SMILES: c1ccc(-c2ccc(N(c3ccccc3)c3ccc4c5ccccc5n(-c5ccc(-c6ccccc6)cc5-c5cccc6c5oc5ccccc56)c4c3)cc2)cc1 ; Cc1ccc(-n2c3ccccc3c3ccc(N(c4ccccc4)c4ccc5c(c4)C4(c6ccccc6-c6ccccc64)c4ccccc4-5)cc32)c(-c2cccc3ccccc23)c1 ; Cc1cccc(-c2ccc3ccccc3c2)c1-n1c2ccccc2c2ccc(N(c3ccccc3)c3ccc4c(c3)C(C)(C)c3ccccc3-4)cc21 ; c1ccc(-c2ccc(N(c3ccc(-c4ccccc4)cc3)c3ccc4c5ccccc5n(-c5c(-c6ccc7ccccc7c6)ccc6ccccc56)c4c3)cc2)cc1]

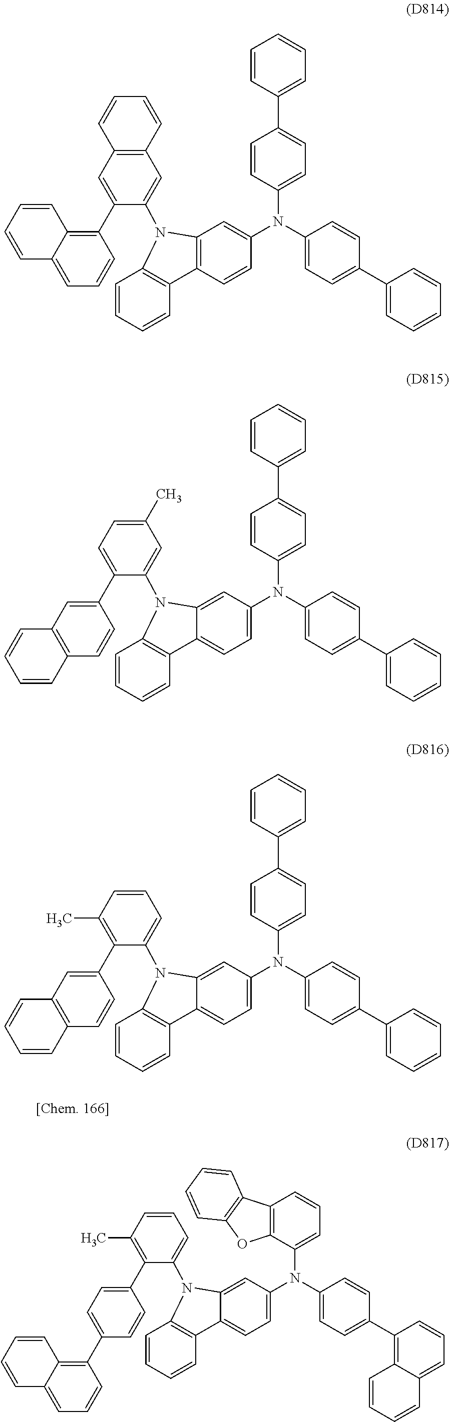 [SMILES: c1ccc(-c2ccc(N(c3ccc(-c4ccccc4)cc3)c3ccc4c5ccccc5n(-c5cc6ccccc6cc5-c5cccc6ccccc56)c4c3)cc2)cc1 ; Cc1ccc(-c2ccc3ccccc3c2)c(-n2c3ccccc3c3ccc(N(c4ccc(-c5ccccc5)cc4)c4ccc(-c5ccccc5)cc4)cc32)c1 ; Cc1cccc(-n2c3ccccc3c3ccc(N(c4ccc(-c5ccccc5)cc4)c4ccc(-c5ccccc5)cc4)cc32)c1-c1ccc2ccccc2c1 ; Cc1cccc(-n2c3ccccc3c3ccc(N(c4ccc(-c5cccc6ccccc56)cc4)c4cccc5c4oc4ccccc45)cc32)c1-c1ccc(-c2cccc3ccccc23)cc1]

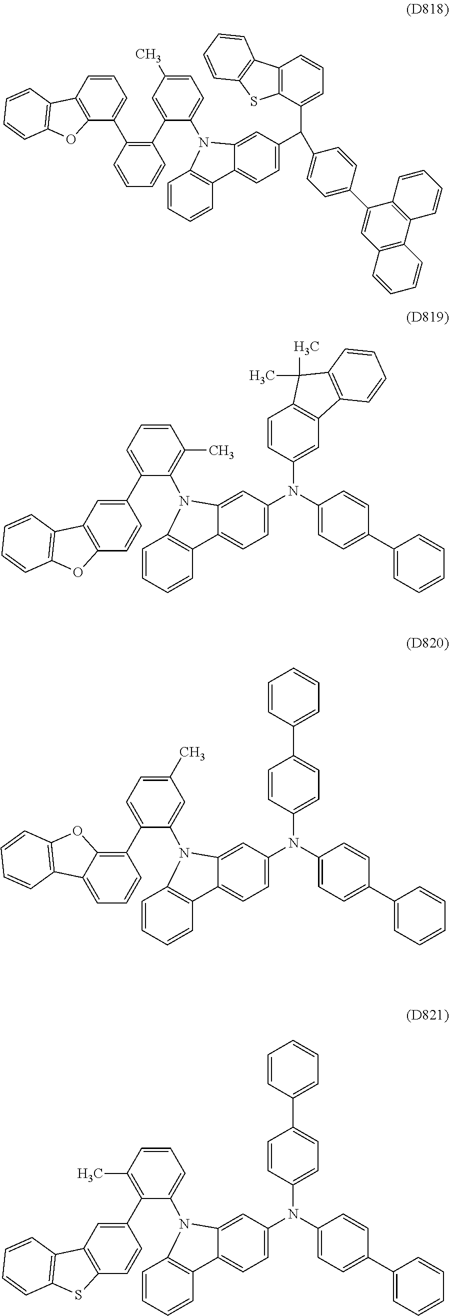 [SMILES: Cc1ccc(-n2c3ccccc3c3ccc(C(c4ccc(-c5cc6ccccc6c6ccccc56)cc4)c4cccc5c4sc4ccccc45)cc32)c(-c2ccccc2-c2cccc3c2oc2ccccc23)c1 ; Cc1cccc(-c2ccc3oc4ccccc4c3c2)c1-n1c2ccccc2c2ccc(N(c3ccc(-c4ccccc4)cc3)c3ccc4c(c3)-c3ccccc3C4(C)C)cc21 ; Cc1ccc(-c2cccc3c2oc2ccccc23)c(-n2c3ccccc3c3ccc(N(c4ccc(-c5ccccc5)cc4)c4ccc(-c5ccccc5)cc4)cc32)c1 ; Cc1cccc(-n2c3ccccc3c3ccc(N(c4ccc(-c5ccccc5)cc4)c4ccc(-c5ccccc5)cc4)cc32)c1-c1ccc2sc3ccccc3c2c1]

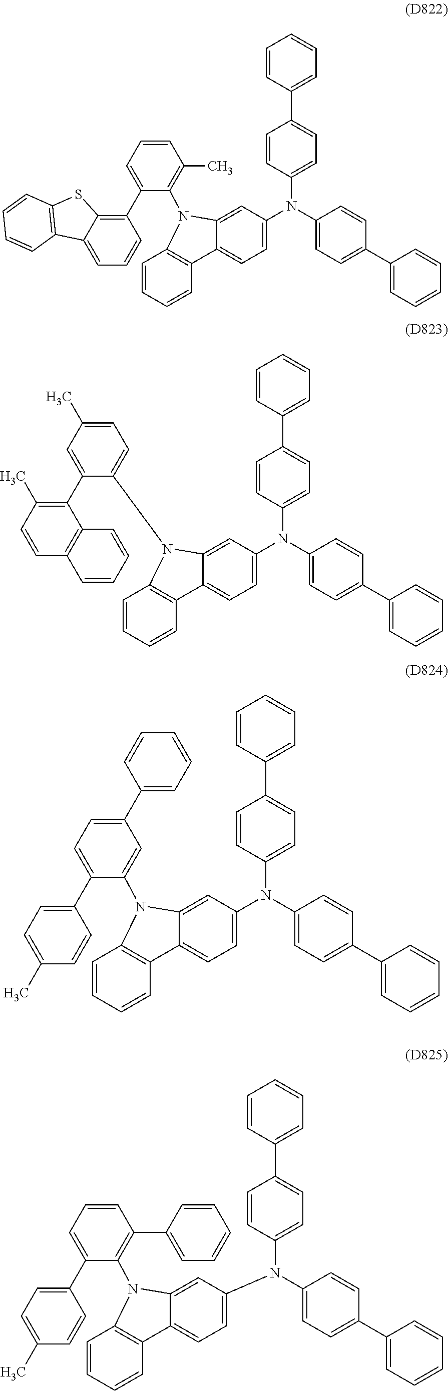 [SMILES: Cc1cccc(-c2cccc3c2sc2ccccc23)c1-n1c2ccccc2c2ccc(N(c3ccc(-c4ccccc4)cc3)c3ccc(-c4ccccc4)cc3)cc21 ; Cc1ccc(-n2c3ccccc3c3ccc(N(c4ccc(-c5ccccc5)cc4)c4ccc(-c5ccccc5)cc4)cc32)c(-c2c(C)ccc3ccccc23)c1 ; Cc1ccc(-c2ccc(-c3ccccc3)cc2-n2c3ccccc3c3ccc(N(c4ccc(-c5ccccc5)cc4)c4ccc(-c5ccccc5)cc4)cc32)cc1 ; Cc1ccc(-c2cccc(-c3ccccc3)c2-n2c3ccccc3c3ccc(N(c4ccc(-c5ccccc5)cc4)c4ccc(-c5ccccc5)cc4)cc32)cc1]

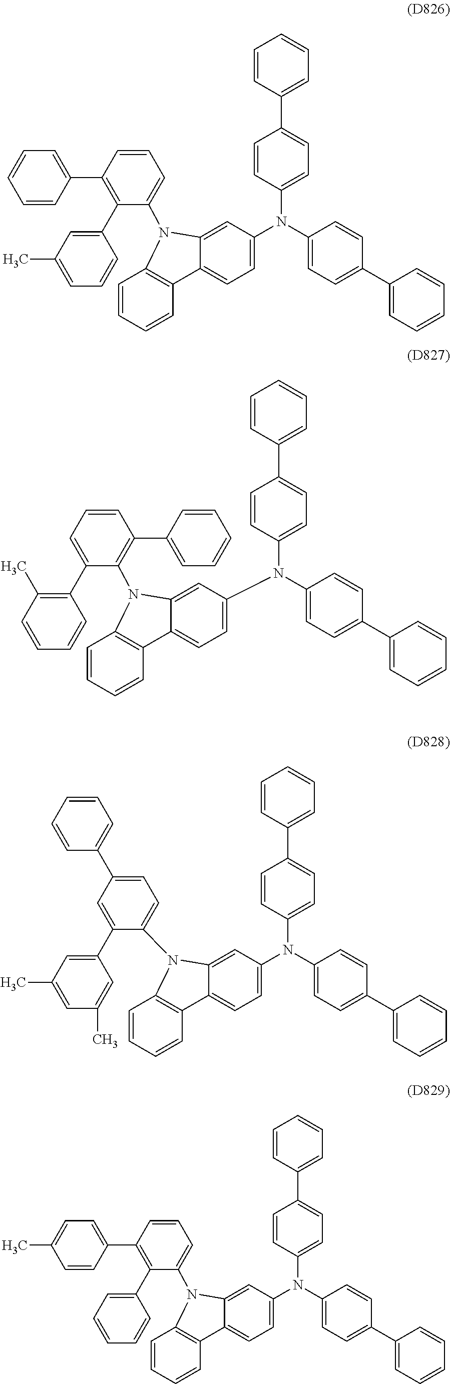 [SMILES: Cc1cccc(-c2c(-c3ccccc3)cccc2-n2c3ccccc3c3ccc(N(c4ccc(-c5ccccc5)cc4)c4ccc(-c5ccccc5)cc4)cc32)c1 ; Cc1ccccc1-c1cccc(-c2ccccc2)c1-n1c2ccccc2c2ccc(N(c3ccc(-c4ccccc4)cc3)c3ccc(-c4ccccc4)cc3)cc21 ; Cc1cc(C)cc(-c2cc(-c3ccccc3)ccc2-n2c3ccccc3c3ccc(N(c4ccc(-c5ccccc5)cc4)c4ccc(-c5ccccc5)cc4)cc32)c1 ; Cc1ccc(-c2cccc(-n3c4ccccc4c4ccc(N(c5ccc(-c6ccccc6)cc5)c5ccc(-c6ccccc6)cc5)cc43)c2-c2ccccc2)cc1]

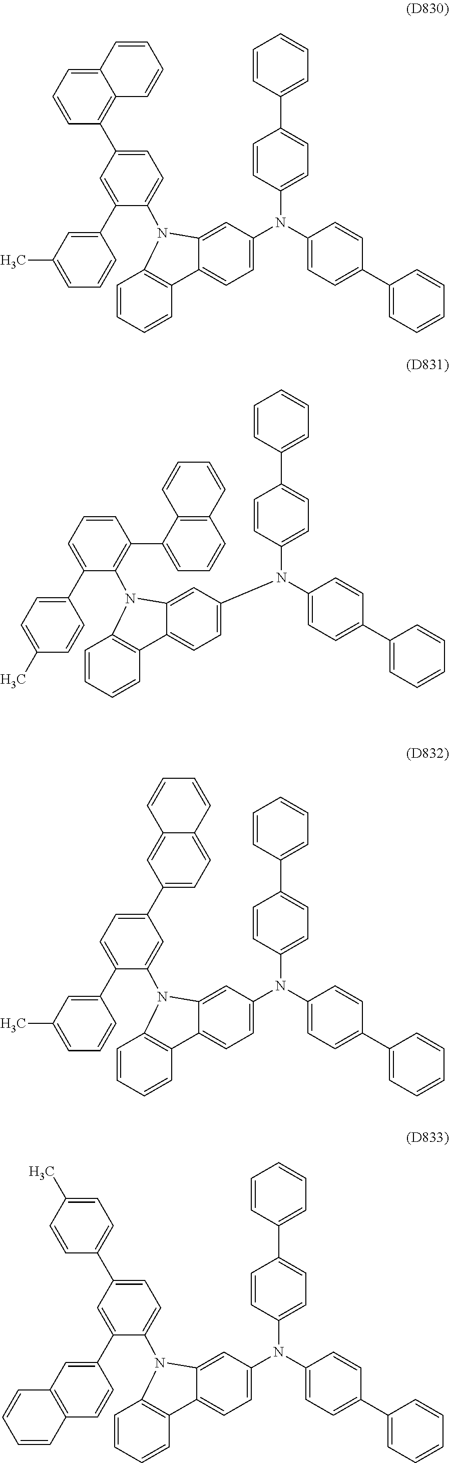 [SMILES: Cc1cccc(-c2cc(-c3cccc4ccccc34)ccc2-n2c3ccccc3c3ccc(N(c4ccc(-c5ccccc5)cc4)c4ccc(-c5ccccc5)cc4)cc32)c1 ; Cc1ccc(-c2cccc(-c3cccc4ccccc34)c2-n2c3ccccc3c3ccc(N(c4ccc(-c5ccccc5)cc4)c4ccc(-c5ccccc5)cc4)cc32)cc1 ; Cc1cccc(-c2ccc(-c3ccc4ccccc4c3)cc2-n2c3ccccc3c3ccc(N(c4ccc(-c5ccccc5)cc4)c4ccc(-c5ccccc5)cc4)cc32)c1 ; Cc1ccc(-c2ccc(-n3c4ccccc4c4ccc(N(c5ccc(-c6ccccc6)cc5)c5ccc(-c6ccccc6)cc5)cc43)c(-c3ccc4ccccc4c3)c2)cc1]

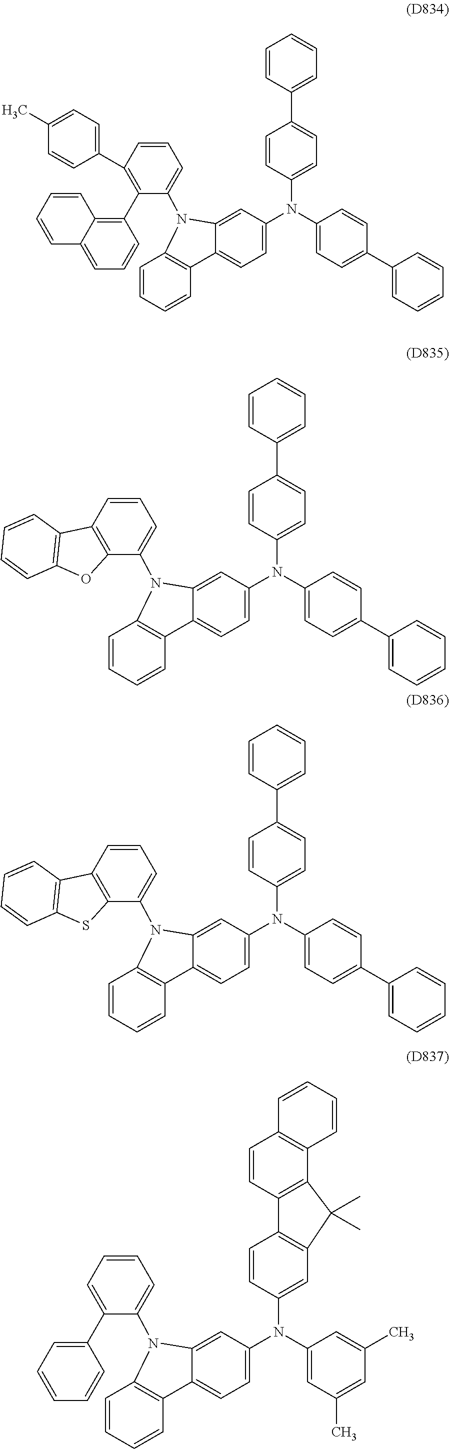 [SMILES: Cc1ccc(-c2cccc(-n3c4ccccc4c4ccc(N(c5ccc(-c6ccccc6)cc5)c5ccc(-c6ccccc6)cc5)cc43)c2-c2cccc3ccccc23)cc1 ; c1ccc(-c2ccc(N(c3ccc(-c4ccccc4)cc3)c3ccc4c5ccccc5n(-c5cccc6c5oc5ccccc56)c4c3)cc2)cc1 ; c1ccc(-c2ccc(N(c3ccc(-c4ccccc4)cc3)c3ccc4c5ccccc5n(-c5cccc6c5sc5ccccc56)c4c3)cc2)cc1 ; Cc1cc(C)cc(N(c2ccc3c(c2)C(C)(C)c2c-3ccc3ccccc23)c2ccc3c4ccccc4n(-c4ccccc4-c4ccccc4)c3c2)c1]

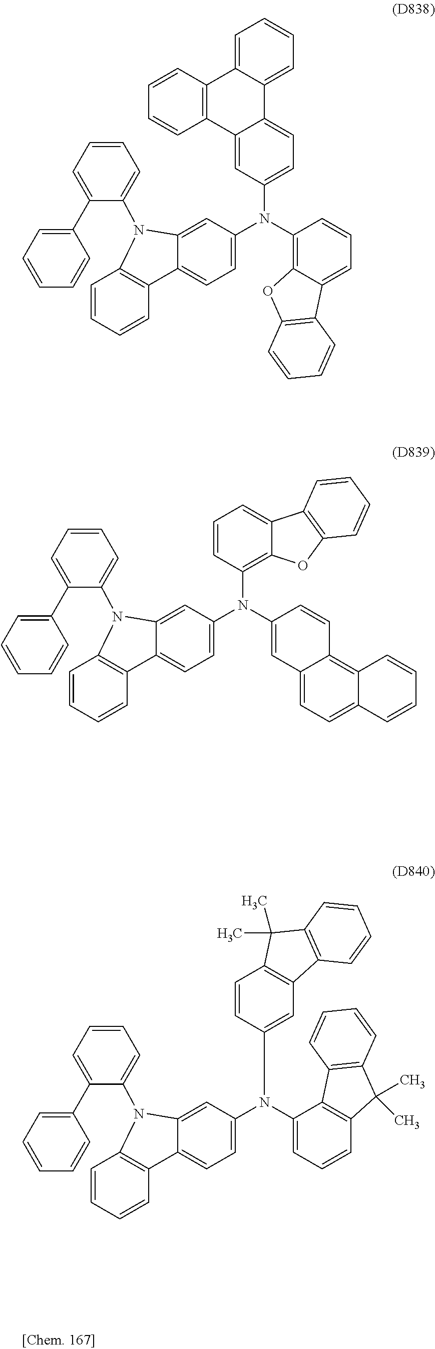 [SMILES: c1ccc(-c2ccccc2-n2c3ccccc3c3ccc(N(c4ccc5c6ccccc6c6ccccc6c5c4)c4cccc5c4oc4ccccc45)cc32)cc1 ; c1ccc(-c2ccccc2-n2c3ccccc3c3ccc(N(c4ccc5c(ccc6ccccc65)c4)c4cccc5c4oc4ccccc45)cc32)cc1 ; CC1(C)c2ccccc2-c2cc(N(c3ccc4c5ccccc5n(-c5ccccc5-c5ccccc5)c4c3)c3cccc4c3-c3ccccc3C4(C)C)ccc21]

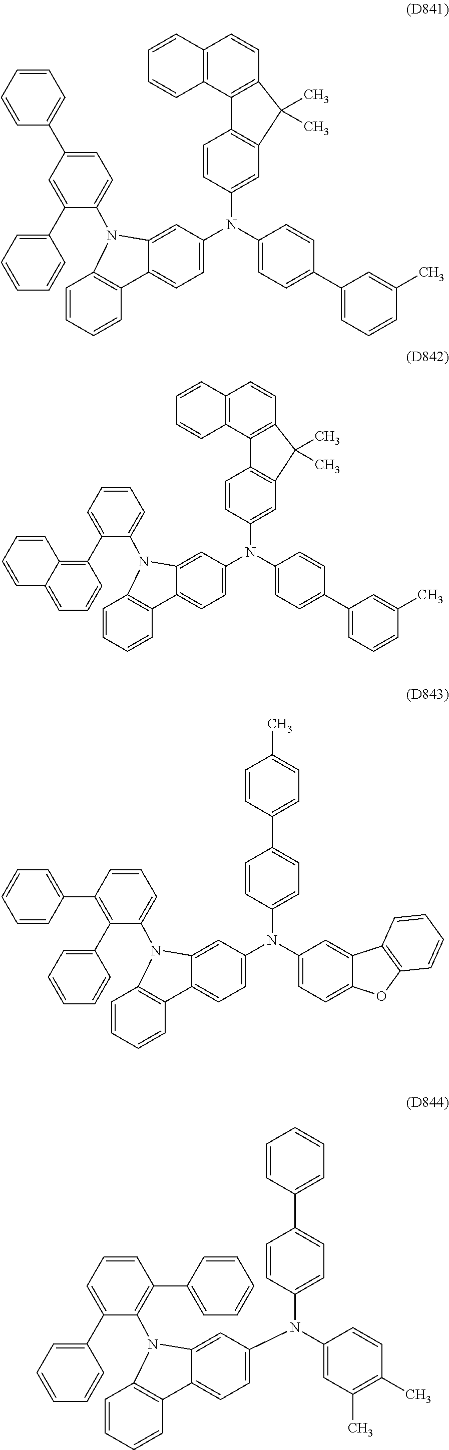 [SMILES: Cc1cccc(-c2ccc(N(c3ccc4c(c3)C(C)(C)c3ccc5ccccc5c3-4)c3ccc4c5ccccc5n(-c5ccc(-c6ccccc6)cc5-c5ccccc5)c4c3)cc2)c1 ; Cc1cccc(-c2ccc(N(c3ccc4c(c3)C(C)(C)c3ccc5ccccc5c3-4)c3ccc4c5ccccc5n(-c5ccccc5-c5cccc6ccccc56)c4c3)cc2)c1 ; Cc1ccc(-c2ccc(N(c3ccc4oc5ccccc5c4c3)c3ccc4c5ccccc5n(-c5cccc(-c6ccccc6)c5-c5ccccc5)c4c3)cc2)cc1 ; Cc1ccc(N(c2ccc(-c3ccccc3)cc2)c2ccc3c4ccccc4n(-c4c(-c5ccccc5)cccc4-c4ccccc4)c3c2)cc1C]

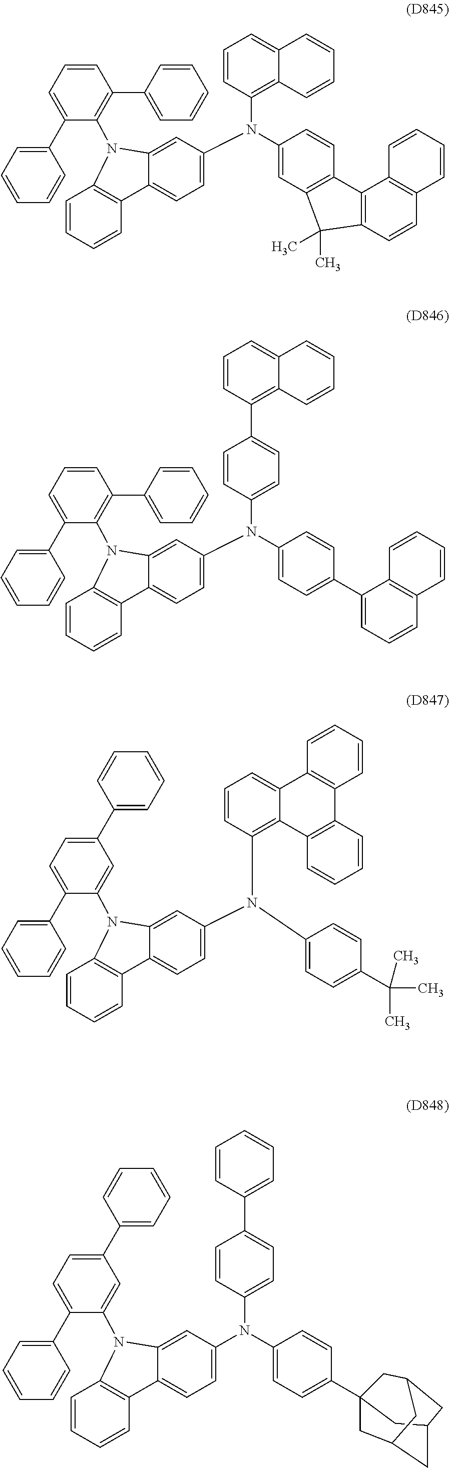 [SMILES: CC1(C)c2cc(N(c3ccc4c5ccccc5n(-c5c(-c6ccccc6)cccc5-c5ccccc5)c4c3)c3cccc4ccccc34)ccc2-c2c1ccc1ccccc21 ; c1ccc(-c2cccc(-c3ccccc3)c2-n2c3ccccc3c3ccc(N(c4ccc(-c5cccc6ccccc56)cc4)c4ccc(-c5cccc6ccccc56)cc4)cc32)cc1 ; CC(C)(C)c1ccc(N(c2ccc3c4ccccc4n(-c4cc(-c5ccccc5)ccc4-c4ccccc4)c3c2)c2cccc3c4ccccc4c4ccccc4c23)cc1 ; c1ccc(-c2ccc(N(c3ccc(C45CC6CC(CC(C6)C4)C5)cc3)c3ccc4c5ccccc5n(-c5cc(-c6ccccc6)ccc5-c5ccccc5)c4c3)cc2)cc1]

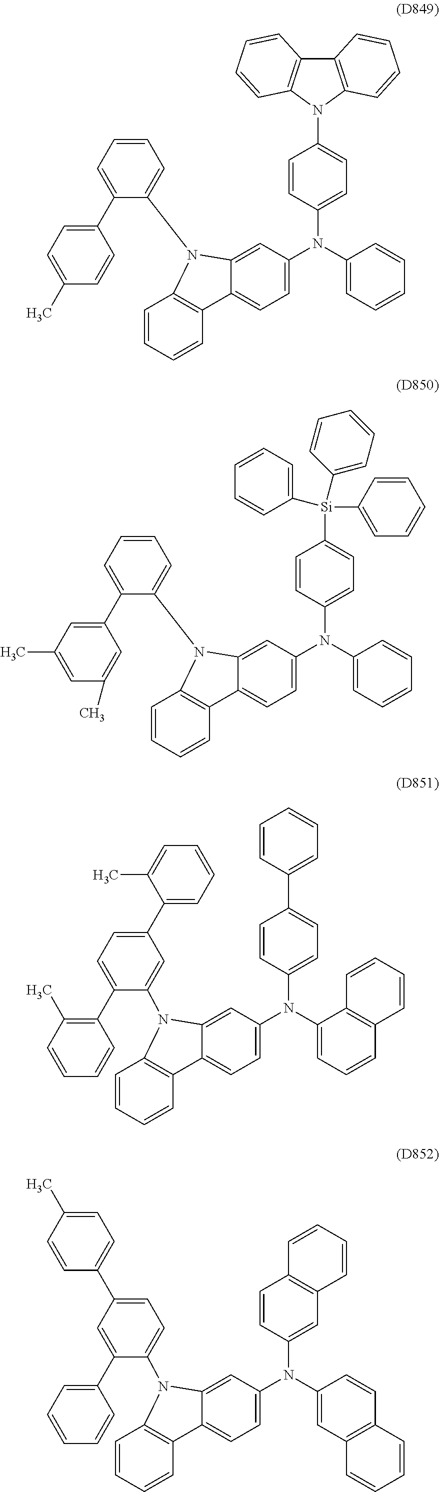 [SMILES: Cc1ccc(-c2ccccc2-n2c3ccccc3c3ccc(N(c4ccccc4)c4ccc(-n5c6ccccc6c6ccccc65)cc4)cc32)cc1 ; Cc1cc(C)cc(-c2ccccc2-n2c3ccccc3c3ccc(N(c4ccccc4)c4ccc([Si](c5ccccc5)(c5ccccc5)c5ccccc5)cc4)cc32)c1 ; Cc1ccccc1-c1ccc(-c2ccccc2C)c(-n2c3ccccc3c3ccc(N(c4ccc(-c5ccccc5)cc4)c4cccc5ccccc45)cc32)c1 ; Cc1ccc(-c2ccc(-n3c4ccccc4c4ccc(N(c5ccc6ccccc6c5)c5ccc6ccccc6c5)cc43)c(-c3ccccc3)c2)cc1]

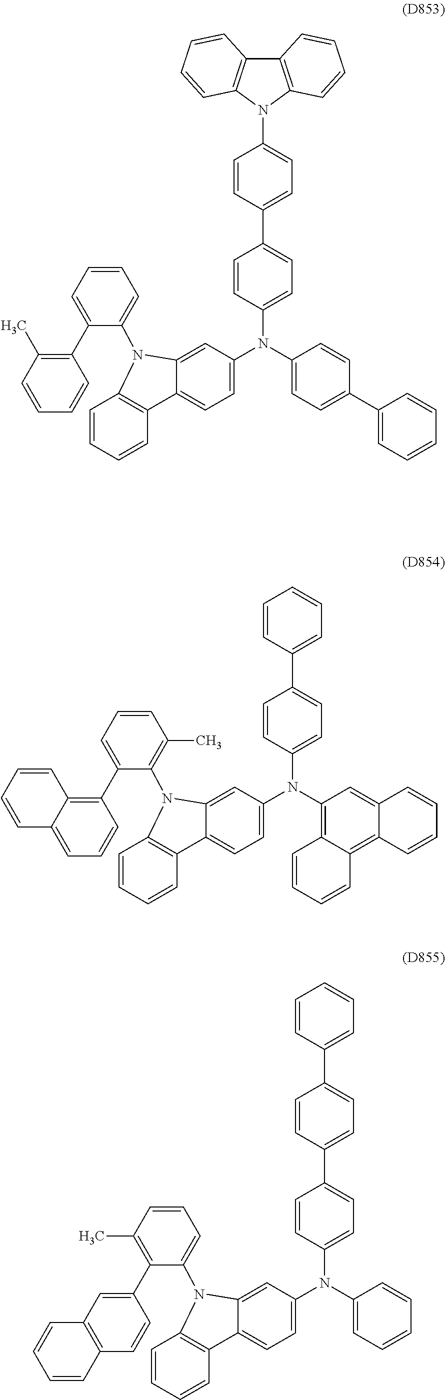 [SMILES: Cc1ccccc1-c1ccccc1-n1c2ccccc2c2ccc(N(c3ccc(-c4ccccc4)cc3)c3ccc(-c4ccc(-n5c6ccccc6c6ccccc65)cc4)cc3)cc21 ; Cc1cccc(-c2cccc3ccccc23)c1-n1c2ccccc2c2ccc(N(c3ccc(-c4ccccc4)cc3)c3cc4ccccc4c4ccccc34)cc21 ; Cc1cccc(-n2c3ccccc3c3ccc(N(c4ccccc4)c4ccc(-c5ccc(-c6ccccc6)cc5)cc4)cc32)c1-c1ccc2ccccc2c1]

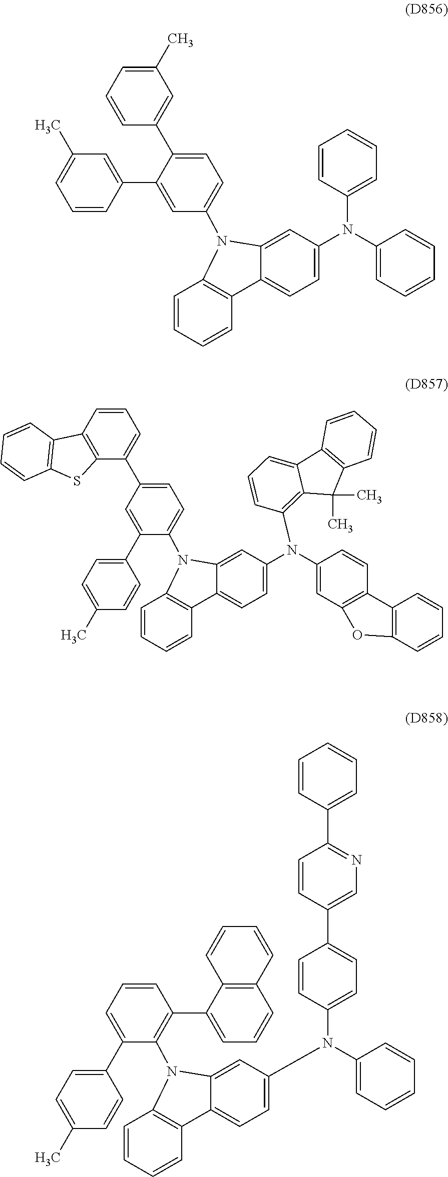 [SMILES: Cc1cccc(-c2ccc(-n3c4ccccc4c4ccc(N(c5ccccc5)c5ccccc5)cc43)cc2-c2cccc(C)c2)c1 ; Cc1ccc(-c2cc(-c3cccc4c3sc3ccccc34)ccc2-n2c3ccccc3c3ccc(N(c4ccc5c(c4)oc4ccccc45)c4cccc5c4C(C)(C)c4ccccc4-5)cc32)cc1 ; Cc1ccc(-c2cccc(-c3cccc4ccccc34)c2-n2c3ccccc3c3ccc(N(c4ccccc4)c4ccc(-c5ccc(-c6ccccc6)nc5)cc4)cc32)cc1]

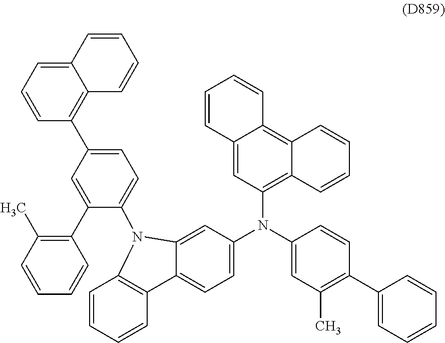 [SMILES: Cc1cc(N(c2ccc3c4ccccc4n(-c4ccc(-c5cccc6ccccc56)cc4-c4ccccc4C)c3c2)c2cc3ccccc3c3ccccc23)ccc1-c1ccccc1]

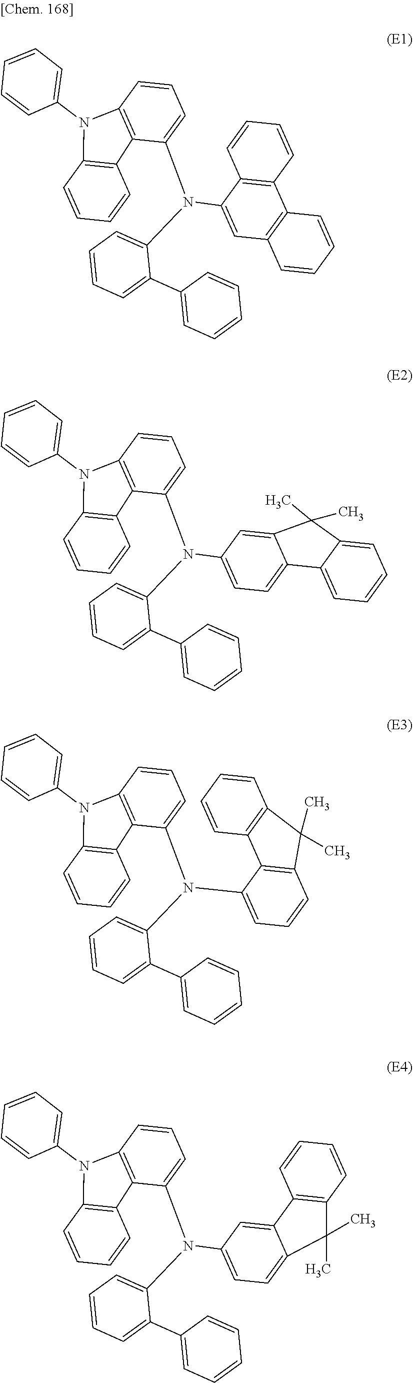 [SMILES: c1ccc(-c2ccccc2N(c2cc3ccccc3c3ccccc23)c2cccc3c2c2ccccc2n3-c2ccccc2)cc1 ; CC1(C)c2ccccc2-c2ccc(N(c3ccccc3-c3ccccc3)c3cccc4c3c3ccccc3n4-c3ccccc3)cc21 ; CC1(C)c2ccccc2-c2c(N(c3ccccc3-c3ccccc3)c3cccc4c3c3ccccc3n4-c3ccccc3)cccc21 ; CC1(C)c2ccccc2-c2cc(N(c3ccccc3-c3ccccc3)c3cccc4c3c3ccccc3n4-c3ccccc3)ccc21]

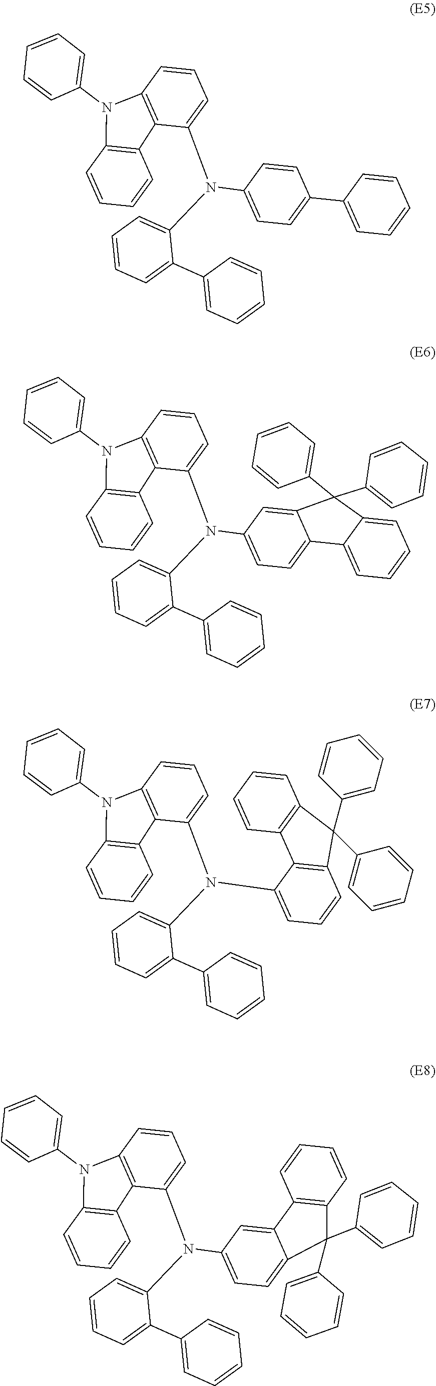 [SMILES: c1ccc(-c2ccc(N(c3ccccc3-c3ccccc3)c3cccc4c3c3ccccc3n4-c3ccccc3)cc2)cc1 ; c1ccc(-c2ccccc2N(c2ccc3c(c2)C(c2ccccc2)(c2ccccc2)c2ccccc2-3)c2cccc3c2c2ccccc2n3-c2ccccc2)cc1 ; c1ccc(-c2ccccc2N(c2cccc3c2-c2ccccc2C3(c2ccccc2)c2ccccc2)c2cccc3c2c2ccccc2n3-c2ccccc2)cc1 ; c1ccc(-c2ccccc2N(c2ccc3c(c2)-c2ccccc2C3(c2ccccc2)c2ccccc2)c2cccc3c2c2ccccc2n3-c2ccccc2)cc1]

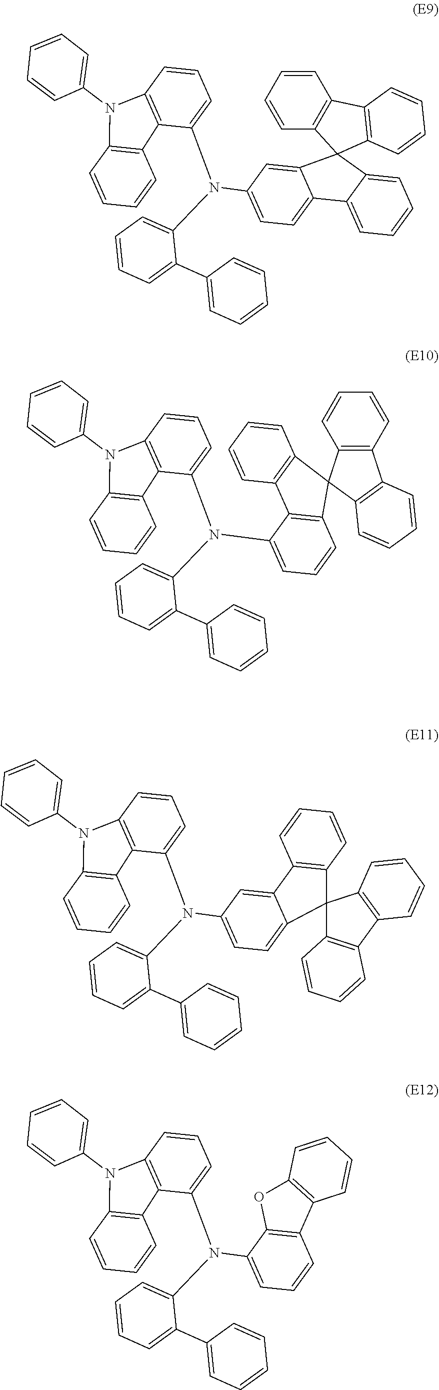 [SMILES: c1ccc(-c2ccccc2N(c2ccc3c(c2)C2(c4ccccc4-c4ccccc42)c2ccccc2-3)c2cccc3c2c2ccccc2n3-c2ccccc2)cc1 ; c1ccc(-c2ccccc2N(c2cccc3c2-c2ccccc2C32c3ccccc3-c3ccccc32)c2cccc3c2c2ccccc2n3-c2ccccc2)cc1 ; c1ccc(-c2ccccc2N(c2ccc3c(c2)-c2ccccc2C32c3ccccc3-c3ccccc32)c2cccc3c2c2ccccc2n3-c2ccccc2)cc1 ; c1ccc(-c2ccccc2N(c2cccc3c2oc2ccccc23)c2cccc3c2c2ccccc2n3-c2ccccc2)cc1]

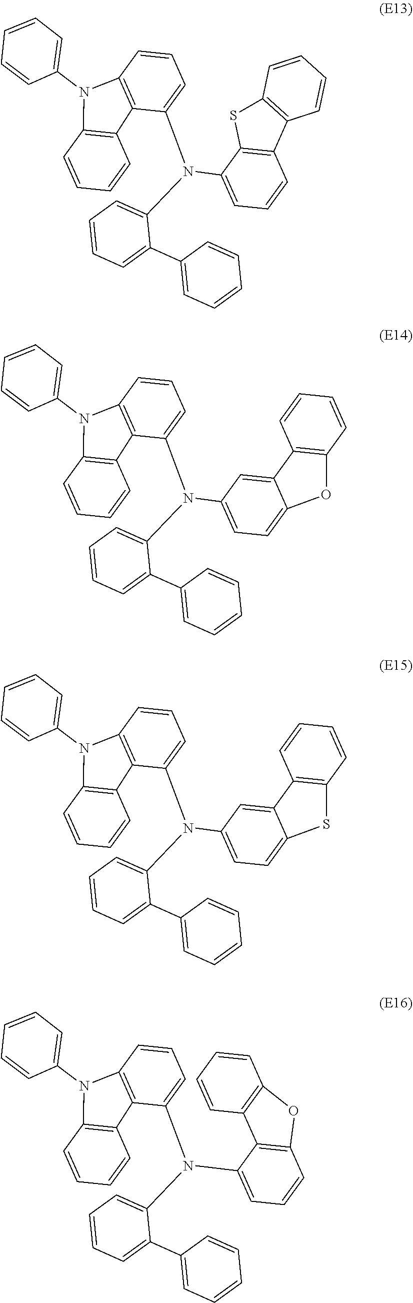 [SMILES: c1ccc(-c2ccccc2N(c2cccc3c2sc2ccccc23)c2cccc3c2c2ccccc2n3-c2ccccc2)cc1 ; c1ccc(-c2ccccc2N(c2ccc3oc4ccccc4c3c2)c2cccc3c2c2ccccc2n3-c2ccccc2)cc1 ; c1ccc(-c2ccccc2N(c2ccc3sc4ccccc4c3c2)c2cccc3c2c2ccccc2n3-c2ccccc2)cc1 ; c1ccc(-c2ccccc2N(c2cccc3oc4ccccc4c23)c2cccc3c2c2ccccc2n3-c2ccccc2)cc1]

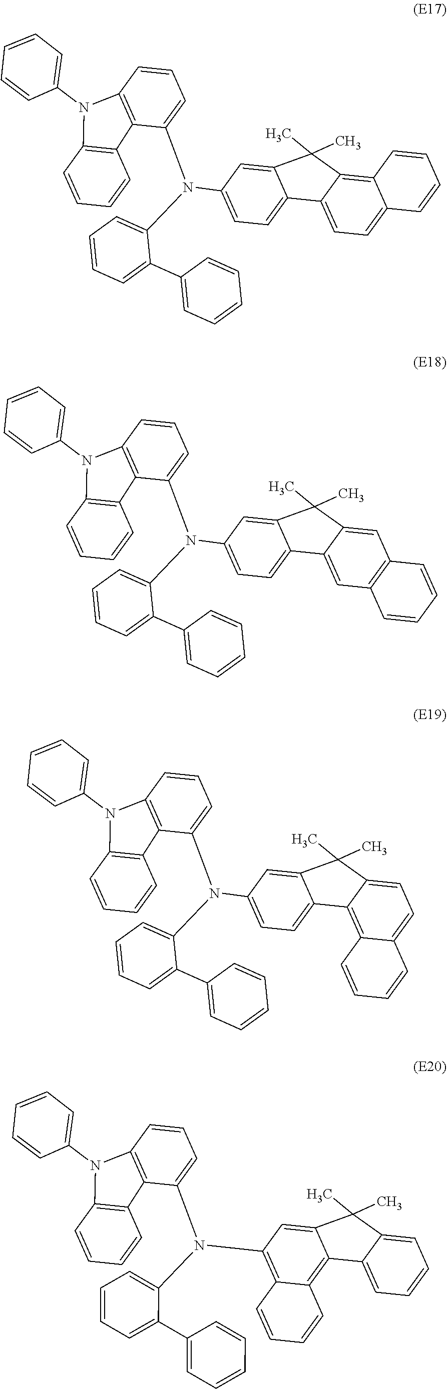 [SMILES: CC1(C)c2cc(N(c3ccccc3-c3ccccc3)c3cccc4c3c3ccccc3n4-c3ccccc3)ccc2-c2ccc3ccccc3c21 ; CC1(C)c2cc(N(c3ccccc3-c3ccccc3)c3cccc4c3c3ccccc3n4-c3ccccc3)ccc2-c2cc3ccccc3cc21 ; CC1(C)c2cc(N(c3ccccc3-c3ccccc3)c3cccc4c3c3ccccc3n4-c3ccccc3)ccc2-c2c1ccc1ccccc21 ; CC1(C)c2ccccc2-c2c1cc(N(c1ccccc1-c1ccccc1)c1cccc3c1c1ccccc1n3-c1ccccc1)c1ccccc21]

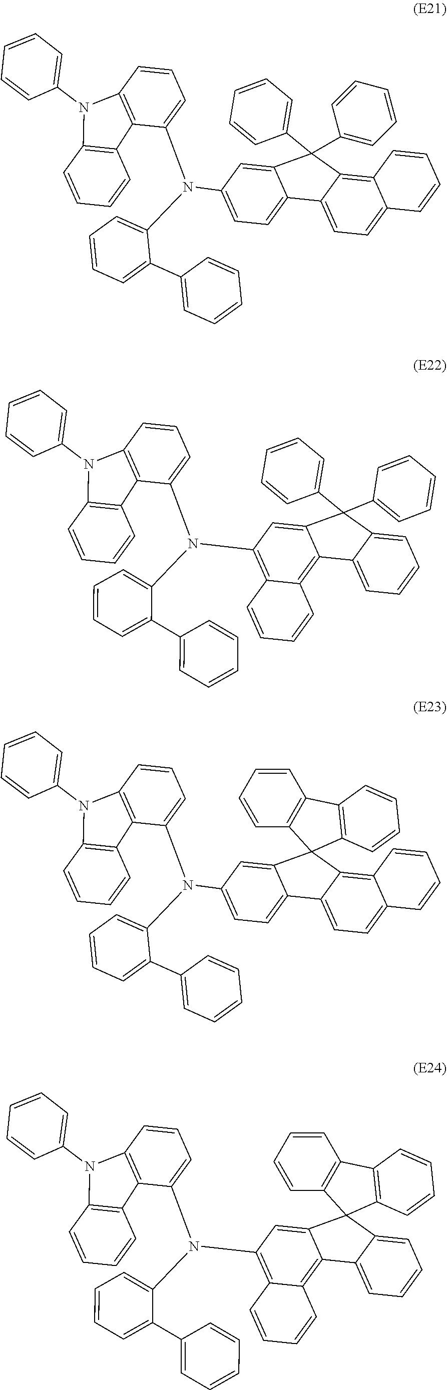 [SMILES: c1ccc(-c2ccccc2N(c2ccc3c(c2)C(c2ccccc2)(c2ccccc2)c2c-3ccc3ccccc23)c2cccc3c2c2ccccc2n3-c2ccccc2)cc1 ; c1ccc(-c2ccccc2N(c2cc3c(c4ccccc24)-c2ccccc2C3(c2ccccc2)c2ccccc2)c2cccc3c2c2ccccc2n3-c2ccccc2)cc1 ; c1ccc(-c2ccccc2N(c2ccc3c(c2)C2(c4ccccc4-c4ccccc42)c2c-3ccc3ccccc23)c2cccc3c2c2ccccc2n3-c2ccccc2)cc1 ; c1ccc(-c2ccccc2N(c2cc3c(c4ccccc24)-c2ccccc2C32c3ccccc3-c3ccccc32)c2cccc3c2c2ccccc2n3-c2ccccc2)cc1]

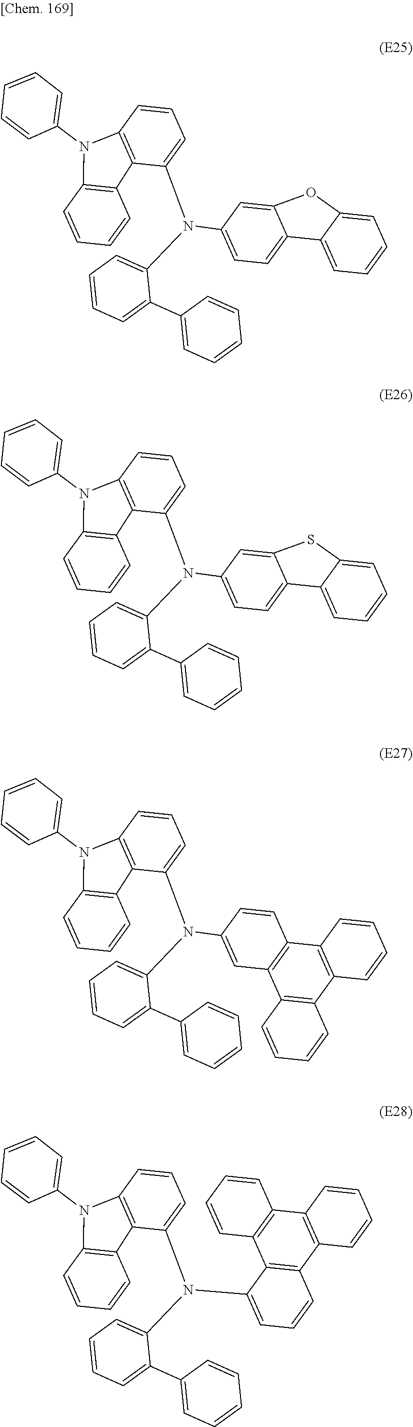 [SMILES: c1ccc(-c2ccccc2N(c2ccc3c(c2)oc2ccccc23)c2cccc3c2c2ccccc2n3-c2ccccc2)cc1 ; c1ccc(-c2ccccc2N(c2ccc3c(c2)sc2ccccc23)c2cccc3c2c2ccccc2n3-c2ccccc2)cc1 ; c1ccc(-c2ccccc2N(c2ccc3c4ccccc4c4ccccc4c3c2)c2cccc3c2c2ccccc2n3-c2ccccc2)cc1 ; c1ccc(-c2ccccc2N(c2cccc3c4ccccc4c4ccccc4c23)c2cccc3c2c2ccccc2n3-c2ccccc2)cc1]

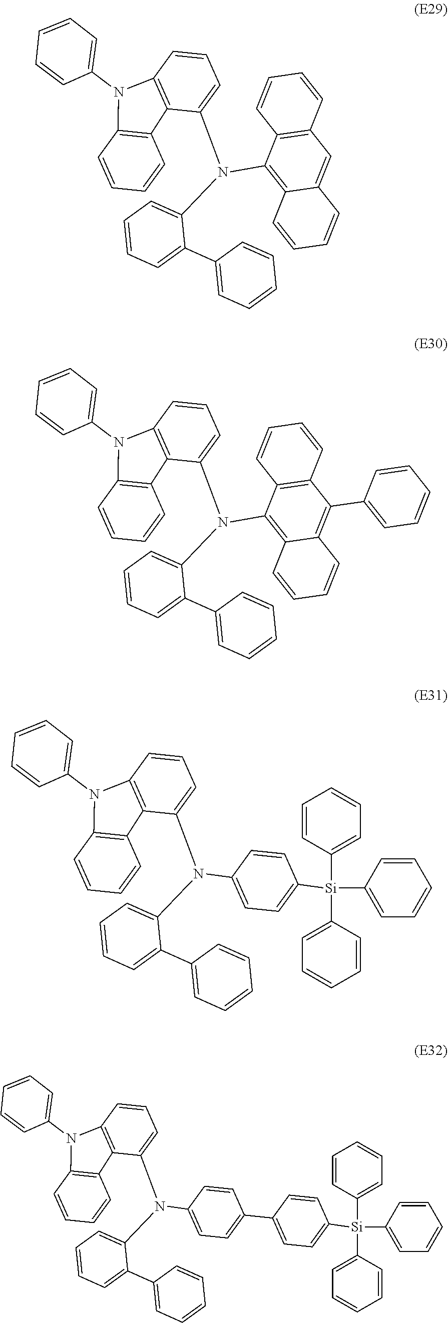 [SMILES: c1ccc(-c2ccccc2N(c2c3ccccc3cc3ccccc23)c2cccc3c2c2ccccc2n3-c2ccccc2)cc1 ; c1ccc(-c2ccccc2N(c2c3ccccc3c(-c3ccccc3)c3ccccc23)c2cccc3c2c2ccccc2n3-c2ccccc2)cc1 ; c1ccc(-c2ccccc2N(c2ccc([Si](c3ccccc3)(c3ccccc3)c3ccccc3)cc2)c2cccc3c2c2ccccc2n3-c2ccccc2)cc1 ; c1ccc(-c2ccccc2N(c2ccc(-c3ccc([Si](c4ccccc4)(c4ccccc4)c4ccccc4)cc3)cc2)c2cccc3c2c2ccccc2n3-c2ccccc2)cc1]

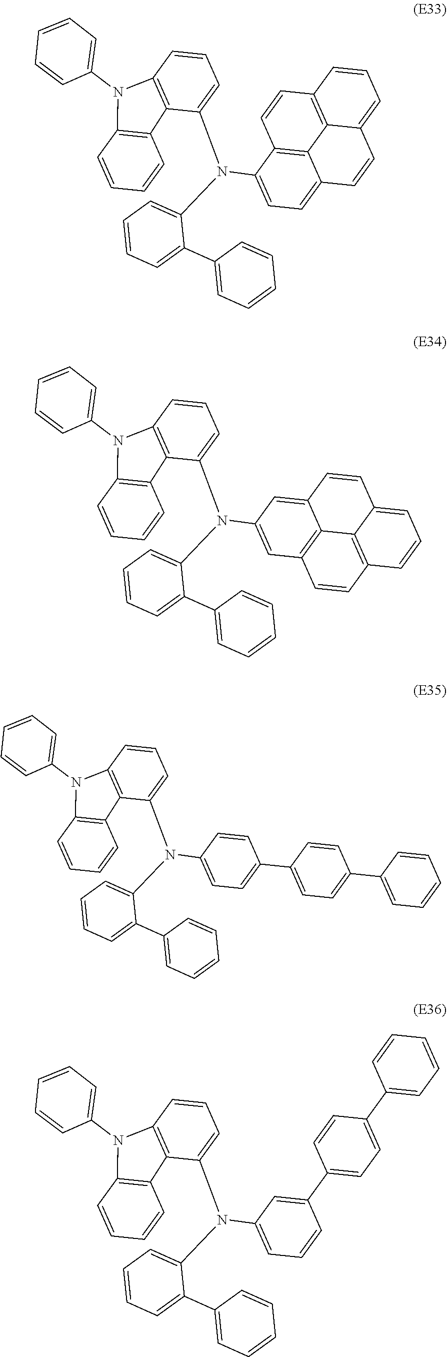 [SMILES: c1ccc(-c2ccccc2N(c2ccc3ccc4cccc5ccc2c3c45)c2cccc3c2c2ccccc2n3-c2ccccc2)cc1 ; c1ccc(-c2ccccc2N(c2cc3ccc4cccc5ccc(c2)c3c45)c2cccc3c2c2ccccc2n3-c2ccccc2)cc1 ; c1ccc(-c2ccc(-c3ccc(N(c4ccccc4-c4ccccc4)c4cccc5c4c4ccccc4n5-c4ccccc4)cc3)cc2)cc1 ; c1ccc(-c2ccc(-c3cccc(N(c4ccccc4-c4ccccc4)c4cccc5c4c4ccccc4n5-c4ccccc4)c3)cc2)cc1]

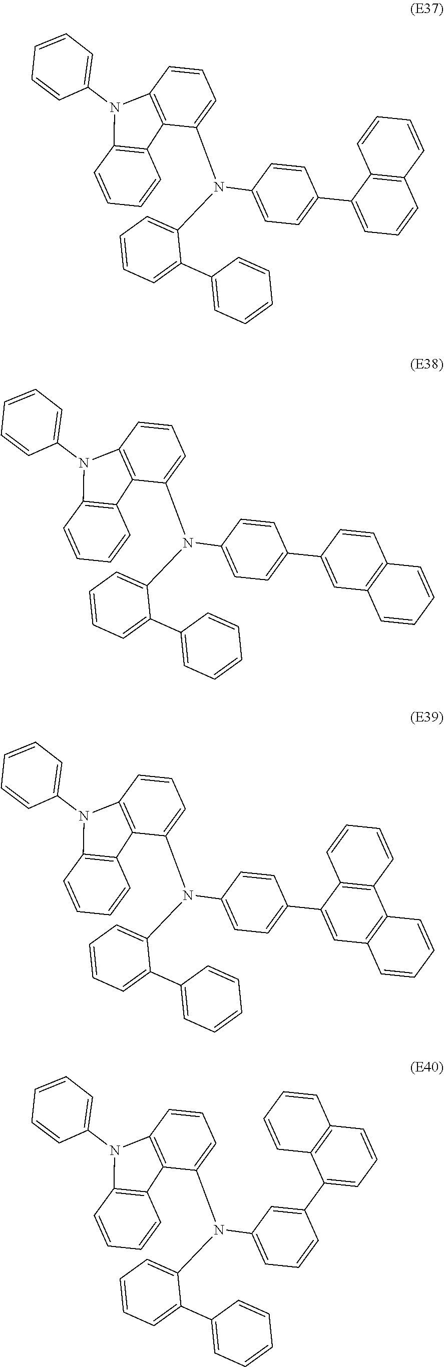 [SMILES: c1ccc(-c2ccccc2N(c2ccc(-c3cccc4ccccc34)cc2)c2cccc3c2c2ccccc2n3-c2ccccc2)cc1 ; c1ccc(-c2ccccc2N(c2ccc(-c3ccc4ccccc4c3)cc2)c2cccc3c2c2ccccc2n3-c2ccccc2)cc1 ; c1ccc(-c2ccccc2N(c2ccc(-c3cc4ccccc4c4ccccc34)cc2)c2cccc3c2c2ccccc2n3-c2ccccc2)cc1 ; c1ccc(-c2ccccc2N(c2cccc(-c3cccc4ccccc34)c2)c2cccc3c2c2ccccc2n3-c2ccccc2)cc1]

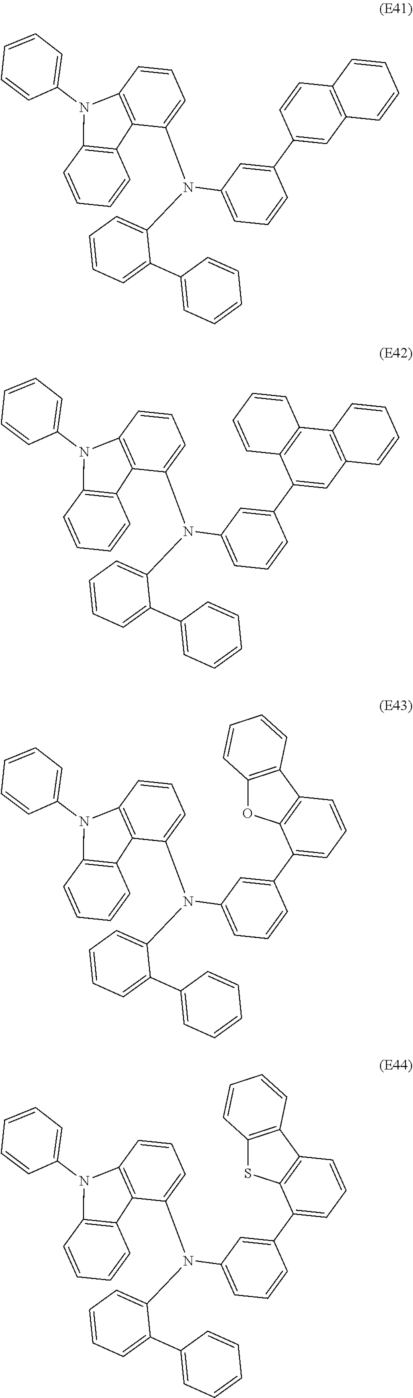 [SMILES: c1ccc(-c2ccccc2N(c2cccc(-c3ccc4ccccc4c3)c2)c2cccc3c2c2ccccc2n3-c2ccccc2)cc1 ; c1ccc(-c2ccccc2N(c2cccc(-c3cc4ccccc4c4ccccc34)c2)c2cccc3c2c2ccccc2n3-c2ccccc2)cc1 ; c1ccc(-c2ccccc2N(c2cccc(-c3cccc4c3oc3ccccc34)c2)c2cccc3c2c2ccccc2n3-c2ccccc2)cc1 ; c1ccc(-c2ccccc2N(c2cccc(-c3cccc4c3sc3ccccc34)c2)c2cccc3c2c2ccccc2n3-c2ccccc2)cc1]

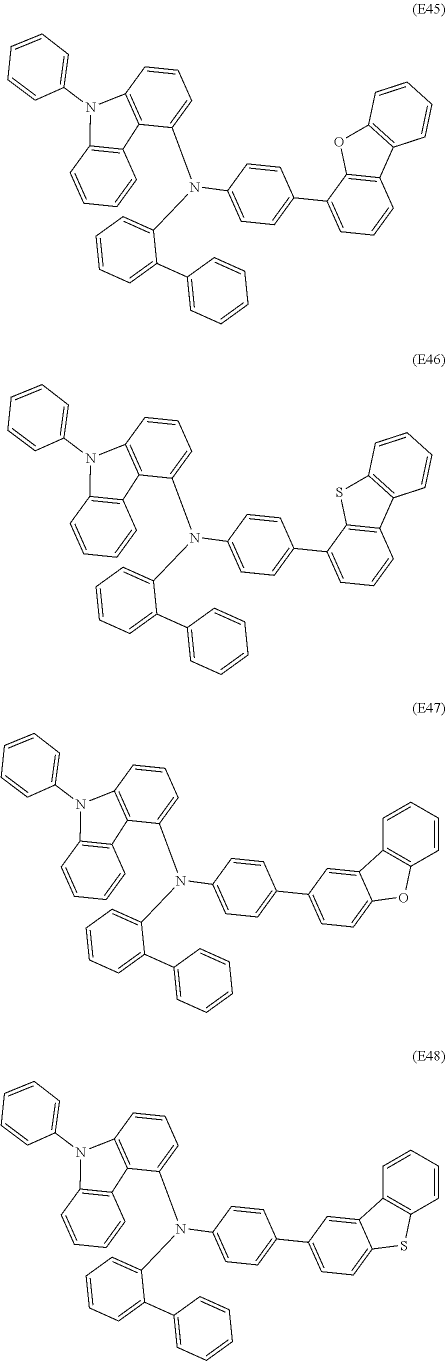 [SMILES: c1ccc(-c2ccccc2N(c2ccc(-c3cccc4c3oc3ccccc34)cc2)c2cccc3c2c2ccccc2n3-c2ccccc2)cc1 ; c1ccc(-c2ccccc2N(c2ccc(-c3cccc4c3sc3ccccc34)cc2)c2cccc3c2c2ccccc2n3-c2ccccc2)cc1 ; c1ccc(-c2ccccc2N(c2ccc(-c3ccc4oc5ccccc5c4c3)cc2)c2cccc3c2c2ccccc2n3-c2ccccc2)cc1 ; c1ccc(-c2ccccc2N(c2ccc(-c3ccc4sc5ccccc5c4c3)cc2)c2cccc3c2c2ccccc2n3-c2ccccc2)cc1]

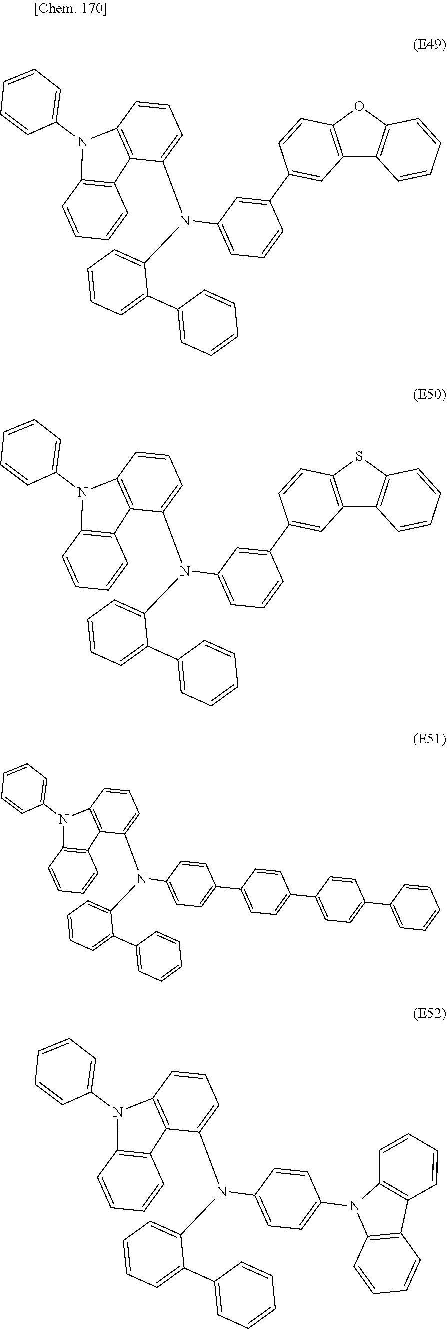 [SMILES: c1ccc(-c2ccccc2N(c2cccc(-c3ccc4oc5ccccc5c4c3)c2)c2cccc3c2c2ccccc2n3-c2ccccc2)cc1 ; c1ccc(-c2ccccc2N(c2cccc(-c3ccc4sc5ccccc5c4c3)c2)c2cccc3c2c2ccccc2n3-c2ccccc2)cc1 ; c1ccc(-c2ccc(-c3ccc(-c4ccc(N(c5ccccc5-c5ccccc5)c5cccc6c5c5ccccc5n6-c5ccccc5)cc4)cc3)cc2)cc1 ; c1ccc(-c2ccccc2N(c2ccc(-n3c4ccccc4c4ccccc43)cc2)c2cccc3c2c2ccccc2n3-c2ccccc2)cc1]

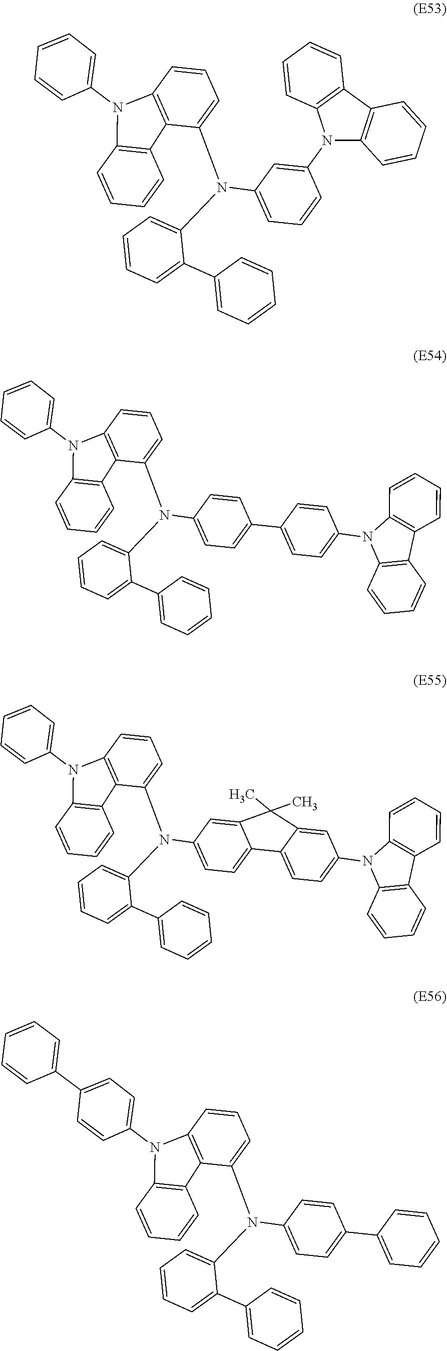 [SMILES: c1ccc(-c2ccccc2N(c2cccc(-n3c4ccccc4c4ccccc43)c2)c2cccc3c2c2ccccc2n3-c2ccccc2)cc1 ; c1ccc(-c2ccccc2N(c2ccc(-c3ccc(-n4c5ccccc5c5ccccc54)cc3)cc2)c2cccc3c2c2ccccc2n3-c2ccccc2)cc1 ; CC1(C)c2cc(N(c3ccccc3-c3ccccc3)c3cccc4c3c3ccccc3n4-c3ccccc3)ccc2-c2ccc(-n3c4ccccc4c4ccccc43)cc21 ; c1ccc(-c2ccc(N(c3ccccc3-c3ccccc3)c3cccc4c3c3ccccc3n4-c3ccc(-c4ccccc4)cc3)cc2)cc1]

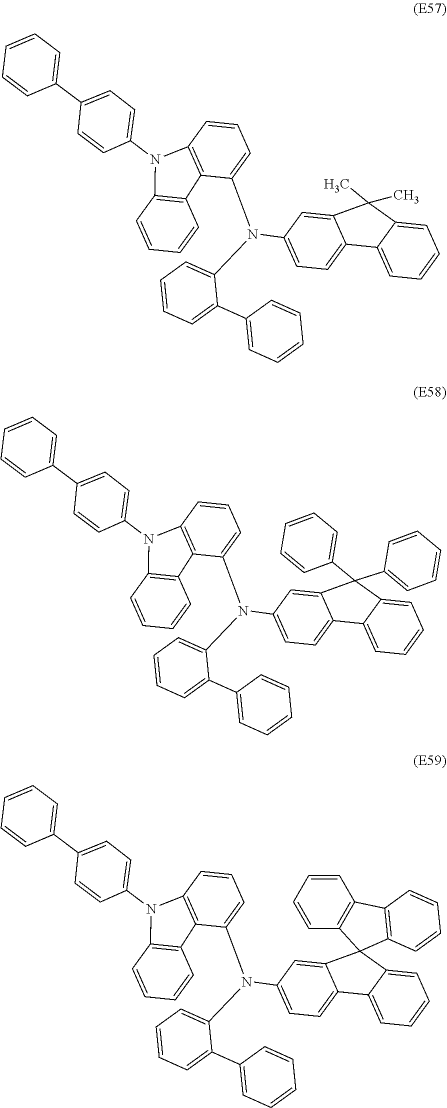 [SMILES: CC1(C)c2ccccc2-c2ccc(N(c3ccccc3-c3ccccc3)c3cccc4c3c3ccccc3n4-c3ccc(-c4ccccc4)cc3)cc21 ; c1ccc(-c2ccc(-n3c4ccccc4c4c(N(c5ccc6c(c5)C(c5ccccc5)(c5ccccc5)c5ccccc5-6)c5ccccc5-c5ccccc5)cccc43)cc2)cc1 ; c1ccc(-c2ccc(-n3c4ccccc4c4c(N(c5ccc6c(c5)C5(c7ccccc7-c7ccccc75)c5ccccc5-6)c5ccccc5-c5ccccc5)cccc43)cc2)cc1]

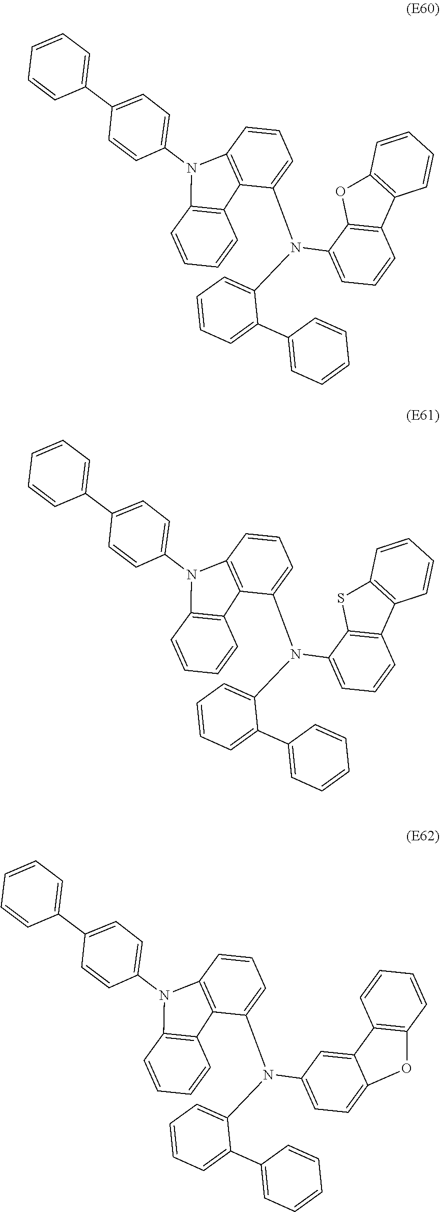 [SMILES: c1ccc(-c2ccc(-n3c4ccccc4c4c(N(c5ccccc5-c5ccccc5)c5cccc6c5oc5ccccc56)cccc43)cc2)cc1 ; c1ccc(-c2ccc(-n3c4ccccc4c4c(N(c5ccccc5-c5ccccc5)c5cccc6c5sc5ccccc56)cccc43)cc2)cc1 ; c1ccc(-c2ccc(-n3c4ccccc4c4c(N(c5ccc6oc7ccccc7c6c5)c5ccccc5-c5ccccc5)cccc43)cc2)cc1]

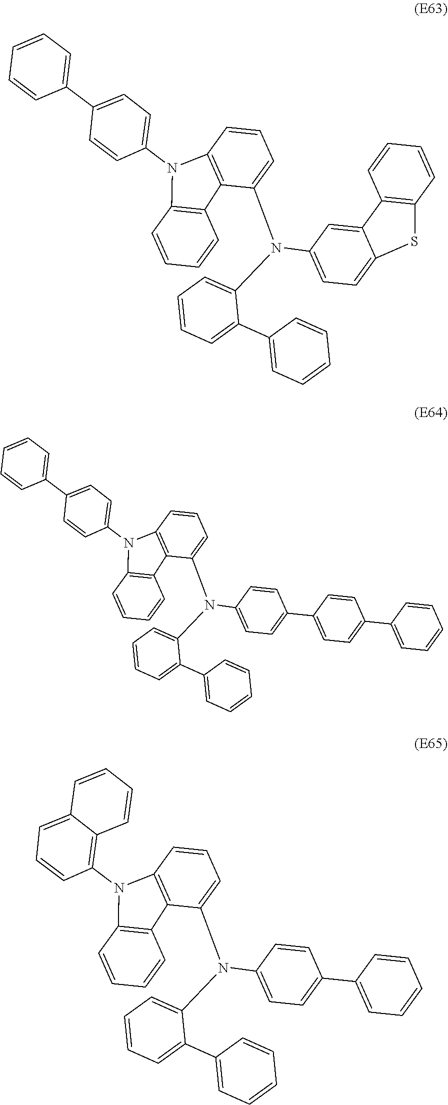 [SMILES: c1ccc(-c2ccc(-n3c4ccccc4c4c(N(c5ccc6sc7ccccc7c6c5)c5ccccc5-c5ccccc5)cccc43)cc2)cc1 ; c1ccc(-c2ccc(-c3ccc(N(c4ccccc4-c4ccccc4)c4cccc5c4c4ccccc4n5-c4ccc(-c5ccccc5)cc4)cc3)cc2)cc1 ; c1ccc(-c2ccc(N(c3ccccc3-c3ccccc3)c3cccc4c3c3ccccc3n4-c3cccc4ccccc34)cc2)cc1]

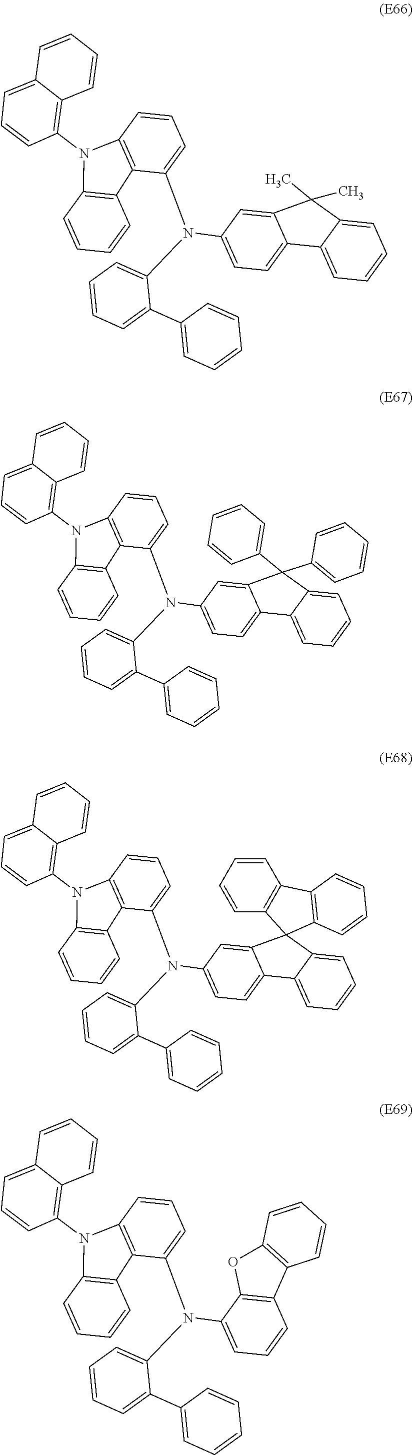 [SMILES: CC1(C)c2ccccc2-c2ccc(N(c3ccccc3-c3ccccc3)c3cccc4c3c3ccccc3n4-c3cccc4ccccc34)cc21 ; c1ccc(-c2ccccc2N(c2ccc3c(c2)C(c2ccccc2)(c2ccccc2)c2ccccc2-3)c2cccc3c2c2ccccc2n3-c2cccc3ccccc23)cc1 ; c1ccc(-c2ccccc2N(c2ccc3c(c2)C2(c4ccccc4-c4ccccc42)c2ccccc2-3)c2cccc3c2c2ccccc2n3-c2cccc3ccccc23)cc1 ; c1ccc(-c2ccccc2N(c2cccc3c2oc2ccccc23)c2cccc3c2c2ccccc2n3-c2cccc3ccccc23)cc1]

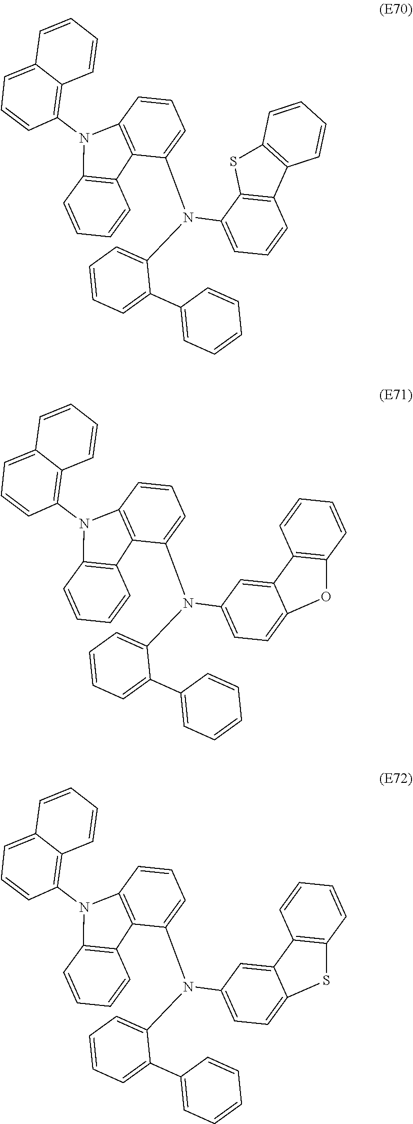 [SMILES: c1ccc(-c2ccccc2N(c2cccc3c2sc2ccccc23)c2cccc3c2c2ccccc2n3-c2cccc3ccccc23)cc1 ; c1ccc(-c2ccccc2N(c2ccc3oc4ccccc4c3c2)c2cccc3c2c2ccccc2n3-c2cccc3ccccc23)cc1 ; c1ccc(-c2ccccc2N(c2ccc3sc4ccccc4c3c2)c2cccc3c2c2ccccc2n3-c2cccc3ccccc23)cc1]

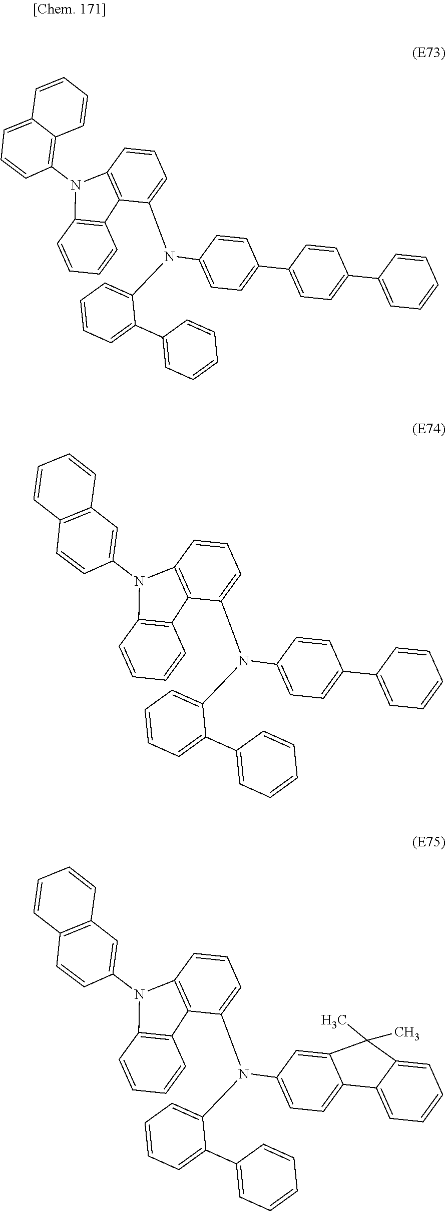 [SMILES: c1ccc(-c2ccc(-c3ccc(N(c4ccccc4-c4ccccc4)c4cccc5c4c4ccccc4n5-c4cccc5ccccc45)cc3)cc2)cc1 ; c1ccc(-c2ccc(N(c3ccccc3-c3ccccc3)c3cccc4c3c3ccccc3n4-c3ccc4ccccc4c3)cc2)cc1 ; CC1(C)c2ccccc2-c2ccc(N(c3ccccc3-c3ccccc3)c3cccc4c3c3ccccc3n4-c3ccc4ccccc4c3)cc21]

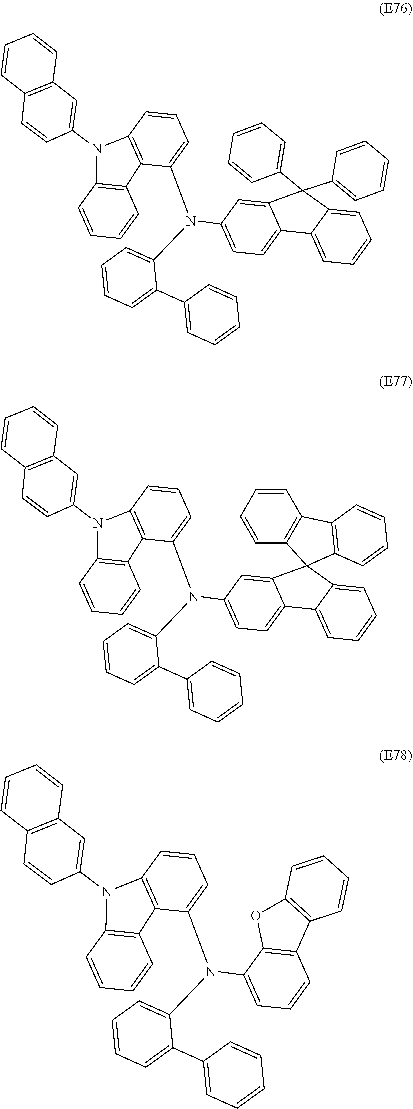 [SMILES: c1ccc(-c2ccccc2N(c2ccc3c(c2)C(c2ccccc2)(c2ccccc2)c2ccccc2-3)c2cccc3c2c2ccccc2n3-c2ccc3ccccc3c2)cc1 ; c1ccc(-c2ccccc2N(c2ccc3c(c2)C2(c4ccccc4-c4ccccc42)c2ccccc2-3)c2cccc3c2c2ccccc2n3-c2ccc3ccccc3c2)cc1 ; c1ccc(-c2ccccc2N(c2cccc3c2oc2ccccc23)c2cccc3c2c2ccccc2n3-c2ccc3ccccc3c2)cc1]

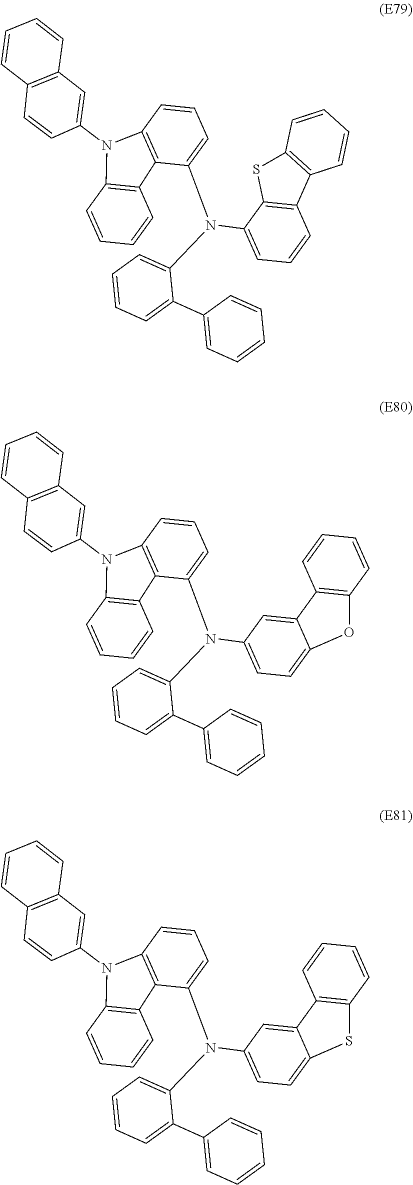 [SMILES: c1ccc(-c2ccccc2N(c2cccc3c2sc2ccccc23)c2cccc3c2c2ccccc2n3-c2ccc3ccccc3c2)cc1 ; c1ccc(-c2ccccc2N(c2ccc3oc4ccccc4c3c2)c2cccc3c2c2ccccc2n3-c2ccc3ccccc3c2)cc1 ; c1ccc(-c2ccccc2N(c2ccc3sc4ccccc4c3c2)c2cccc3c2c2ccccc2n3-c2ccc3ccccc3c2)cc1]

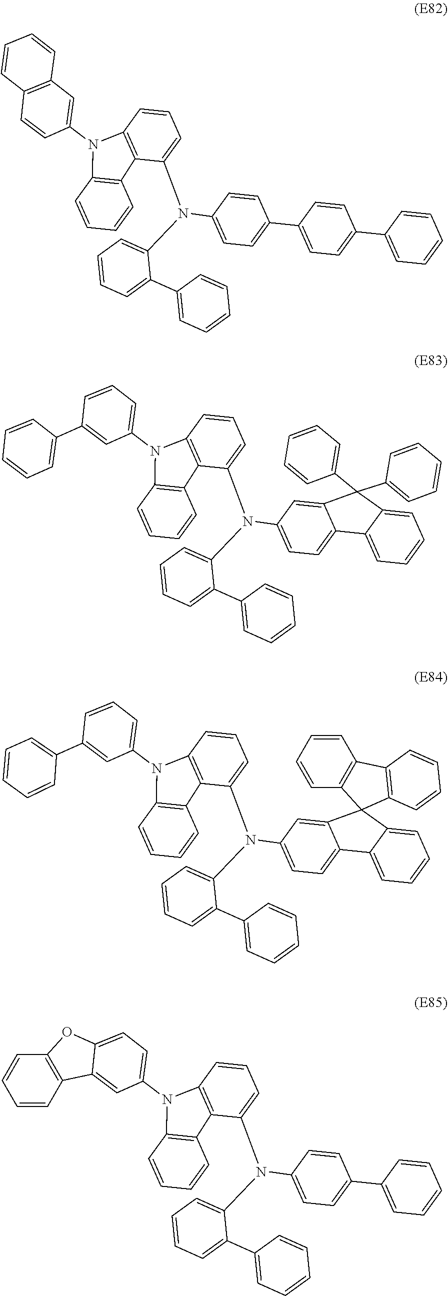 [SMILES: c1ccc(-c2ccc(-c3ccc(N(c4ccccc4-c4ccccc4)c4cccc5c4c4ccccc4n5-c4ccc5ccccc5c4)cc3)cc2)cc1 ; c1ccc(-c2cccc(-n3c4ccccc4c4c(N(c5ccc6c(c5)C(c5ccccc5)(c5ccccc5)c5ccccc5-6)c5ccccc5-c5ccccc5)cccc43)c2)cc1 ; c1ccc(-c2cccc(-n3c4ccccc4c4c(N(c5ccc6c(c5)C5(c7ccccc7-c7ccccc75)c5ccccc5-6)c5ccccc5-c5ccccc5)cccc43)c2)cc1 ; c1ccc(-c2ccc(N(c3ccccc3-c3ccccc3)c3cccc4c3c3ccccc3n4-c3ccc4oc5ccccc5c4c3)cc2)cc1]

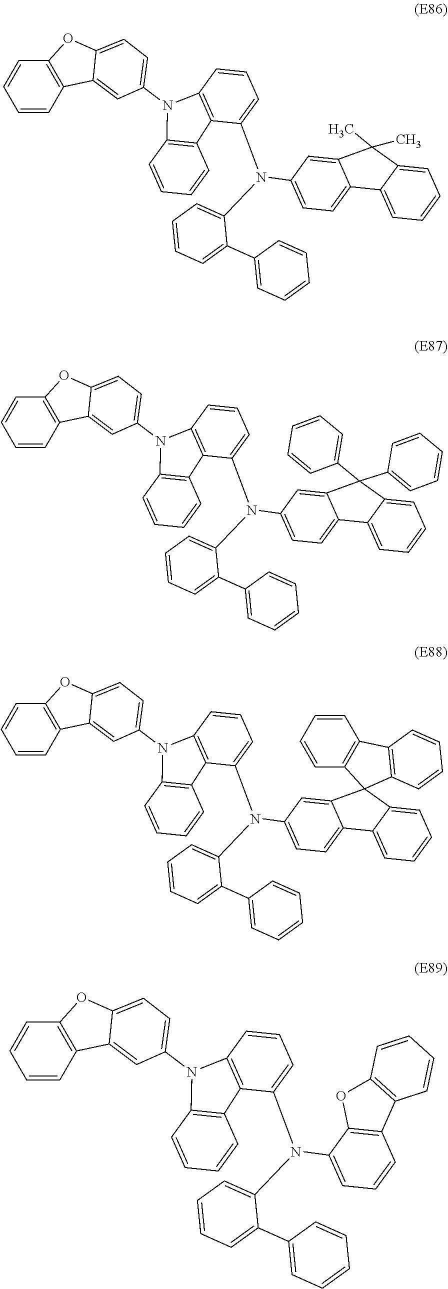 [SMILES: CC1(C)c2ccccc2-c2ccc(N(c3ccccc3-c3ccccc3)c3cccc4c3c3ccccc3n4-c3ccc4oc5ccccc5c4c3)cc21 ; c1ccc(-c2ccccc2N(c2ccc3c(c2)C(c2ccccc2)(c2ccccc2)c2ccccc2-3)c2cccc3c2c2ccccc2n3-c2ccc3oc4ccccc4c3c2)cc1 ; c1ccc(-c2ccccc2N(c2ccc3c(c2)C2(c4ccccc4-c4ccccc42)c2ccccc2-3)c2cccc3c2c2ccccc2n3-c2ccc3oc4ccccc4c3c2)cc1 ; c1ccc(-c2ccccc2N(c2cccc3c2oc2ccccc23)c2cccc3c2c2ccccc2n3-c2ccc3oc4ccccc4c3c2)cc1]

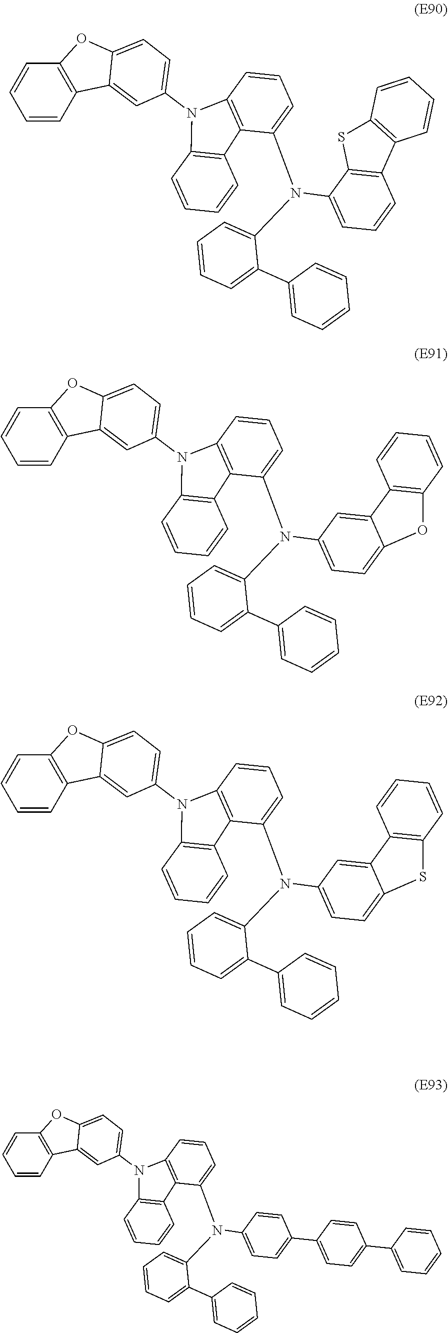 [SMILES: c1ccc(-c2ccccc2N(c2cccc3c2sc2ccccc23)c2cccc3c2c2ccccc2n3-c2ccc3oc4ccccc4c3c2)cc1 ; c1ccc(-c2ccccc2N(c2ccc3oc4ccccc4c3c2)c2cccc3c2c2ccccc2n3-c2ccc3oc4ccccc4c3c2)cc1 ; c1ccc(-c2ccccc2N(c2ccc3sc4ccccc4c3c2)c2cccc3c2c2ccccc2n3-c2ccc3oc4ccccc4c3c2)cc1 ; c1ccc(-c2ccc(-c3ccc(N(c4ccccc4-c4ccccc4)c4cccc5c4c4ccccc4n5-c4ccc5oc6ccccc6c5c4)cc3)cc2)cc1]

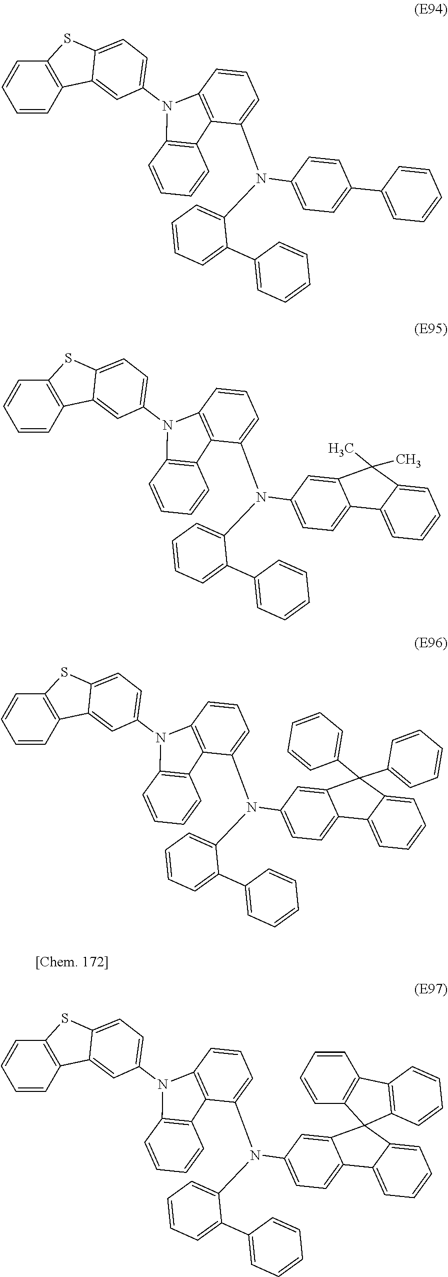 [SMILES: c1ccc(-c2ccc(N(c3ccccc3-c3ccccc3)c3cccc4c3c3ccccc3n4-c3ccc4sc5ccccc5c4c3)cc2)cc1 ; CC1(C)c2ccccc2-c2ccc(N(c3ccccc3-c3ccccc3)c3cccc4c3c3ccccc3n4-c3ccc4sc5ccccc5c4c3)cc21 ; c1ccc(-c2ccccc2N(c2ccc3c(c2)C(c2ccccc2)(c2ccccc2)c2ccccc2-3)c2cccc3c2c2ccccc2n3-c2ccc3sc4ccccc4c3c2)cc1 ; c1ccc(-c2ccccc2N(c2ccc3c(c2)C2(c4ccccc4-c4ccccc42)c2ccccc2-3)c2cccc3c2c2ccccc2n3-c2ccc3sc4ccccc4c3c2)cc1]

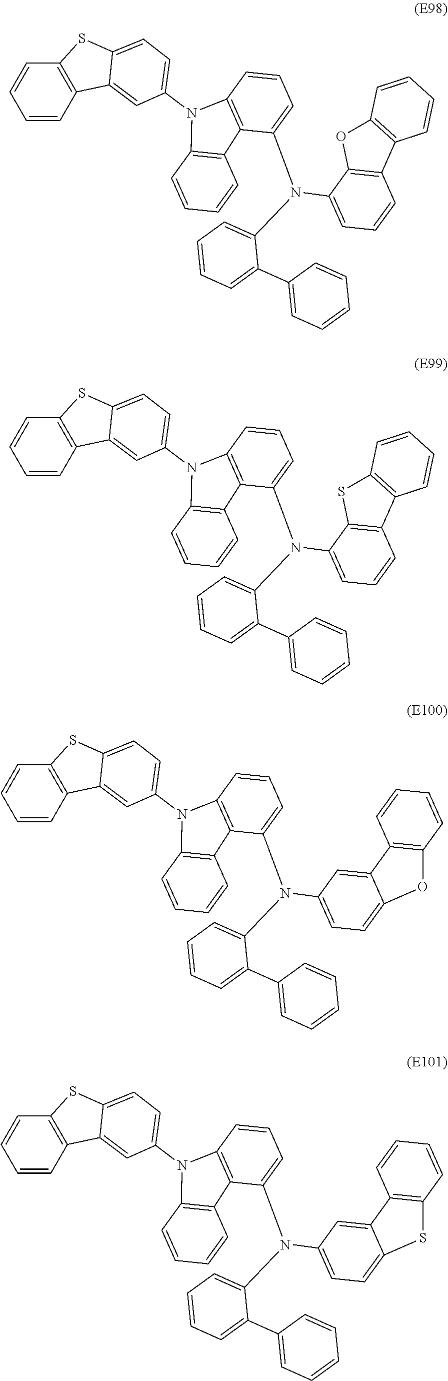 [SMILES: c1ccc(-c2ccccc2N(c2cccc3c2oc2ccccc23)c2cccc3c2c2ccccc2n3-c2ccc3sc4ccccc4c3c2)cc1 ; c1ccc(-c2ccccc2N(c2cccc3c2sc2ccccc23)c2cccc3c2c2ccccc2n3-c2ccc3sc4ccccc4c3c2)cc1 ; c1ccc(-c2ccccc2N(c2ccc3oc4ccccc4c3c2)c2cccc3c2c2ccccc2n3-c2ccc3sc4ccccc4c3c2)cc1 ; c1ccc(-c2ccccc2N(c2ccc3sc4ccccc4c3c2)c2cccc3c2c2ccccc2n3-c2ccc3sc4ccccc4c3c2)cc1]

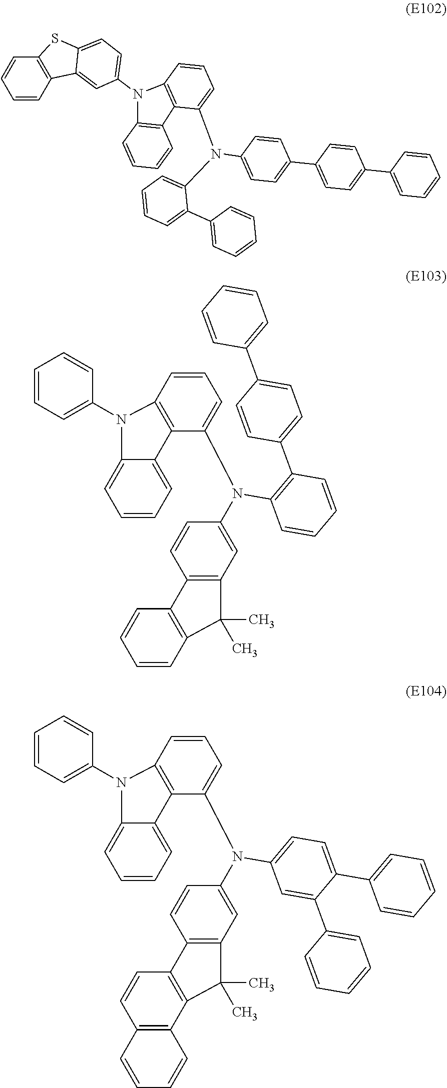

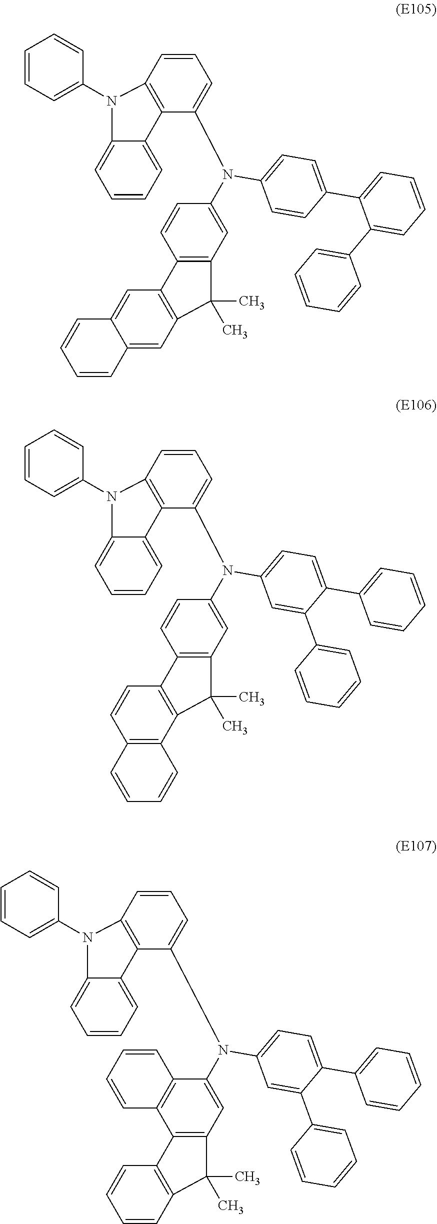 [SMILES: CC1(C)c2cc(N(c3ccc(-c4ccccc4-c4ccccc4)cc3)c3cccc4c3c3ccccc3n4-c3ccccc3)ccc2-c2cc3ccccc3cc21 ; CC1(C)c2cc(N(c3ccc(-c4ccccc4)c(-c4ccccc4)c3)c3cccc4c3c3ccccc3n4-c3ccccc3)ccc2-c2ccc3ccccc3c21 ; CC1(C)c2ccccc2-c2c1cc(N(c1ccc(-c3ccccc3)c(-c3ccccc3)c1)c1cccc3c1c1ccccc1n3-c1ccccc1)c1ccccc21]

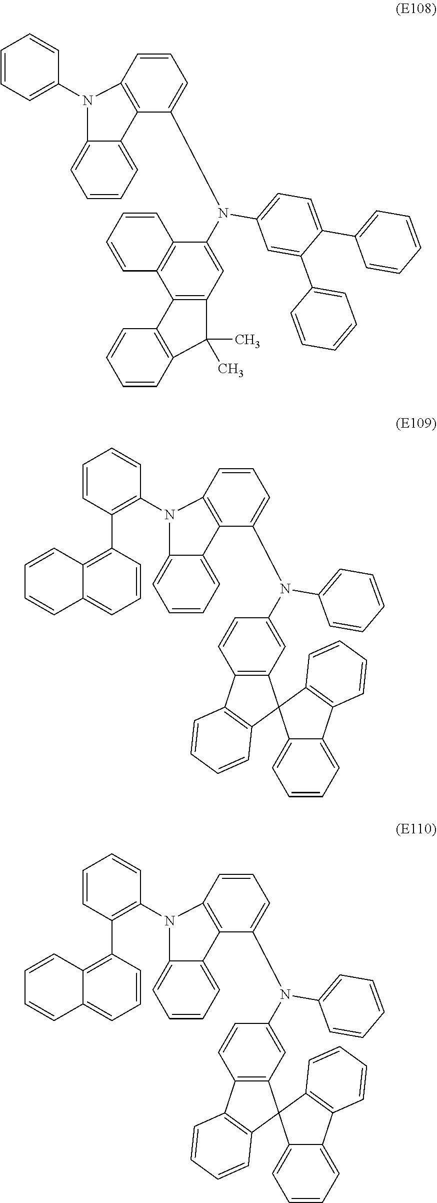 [SMILES: CC1(C)c2ccccc2-c2c1cc(N(c1ccc(-c3ccccc3)c(-c3ccccc3)c1)c1cccc3c1c1ccccc1n3-c1ccccc1)c1ccccc21 ; c1ccc(N(c2ccc3c(c2)C2(c4ccccc4-c4ccccc42)c2ccccc2-3)c2cccc3c2c2ccccc2n3-c2ccccc2-c2cccc3ccccc23)cc1 ; c1ccc(N(c2ccc3c(c2)C2(c4ccccc4-c4ccccc42)c2ccccc2-3)c2cccc3c2c2ccccc2n3-c2ccccc2-c2cccc3ccccc23)cc1]

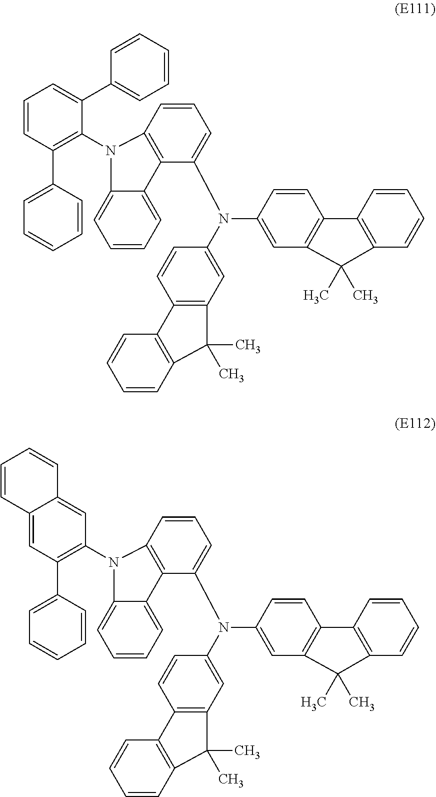 [SMILES: CC1(C)c2ccccc2-c2ccc(N(c3ccc4c(c3)C(C)(C)c3ccccc3-4)c3cccc4c3c3ccccc3n4-c3c(-c4ccccc4)cccc3-c3ccccc3)cc21 ; CC1(C)c2ccccc2-c2ccc(N(c3ccc4c(c3)C(C)(C)c3ccccc3-4)c3cccc4c3c3ccccc3n4-c3cc4ccccc4cc3-c3ccccc3)cc21]

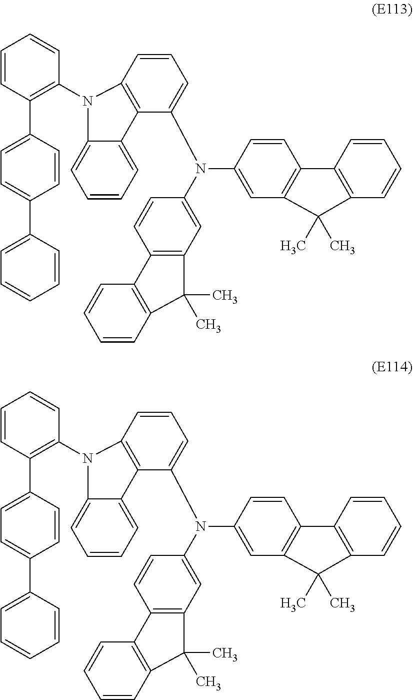 [SMILES: CC1(C)c2ccccc2-c2ccc(N(c3ccc4c(c3)C(C)(C)c3ccccc3-4)c3cccc4c3c3ccccc3n4-c3ccccc3-c3ccc(-c4ccccc4)cc3)cc21 ; CC1(C)c2ccccc2-c2ccc(N(c3ccc4c(c3)C(C)(C)c3ccccc3-4)c3cccc4c3c3ccccc3n4-c3ccccc3-c3ccc(-c4ccccc4)cc3)cc21]

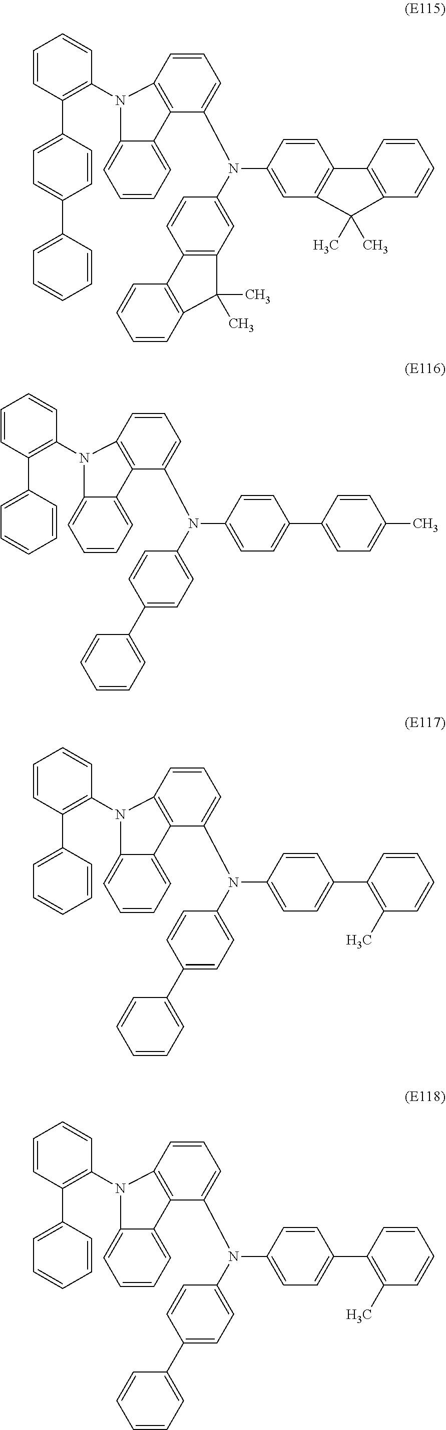 [SMILES: CC1(C)c2ccccc2-c2ccc(N(c3ccc4c(c3)C(C)(C)c3ccccc3-4)c3cccc4c3c3ccccc3n4-c3ccccc3-c3ccc(-c4ccccc4)cc3)cc21 ; Cc1ccc(-c2ccc(N(c3ccc(-c4ccccc4)cc3)c3cccc4c3c3ccccc3n4-c3ccccc3-c3ccccc3)cc2)cc1 ; Cc1ccccc1-c1ccc(N(c2ccc(-c3ccccc3)cc2)c2cccc3c2c2ccccc2n3-c2ccccc2-c2ccccc2)cc1 ; Cc1ccccc1-c1ccc(N(c2ccc(-c3ccccc3)cc2)c2cccc3c2c2ccccc2n3-c2ccccc2-c2ccccc2)cc1]

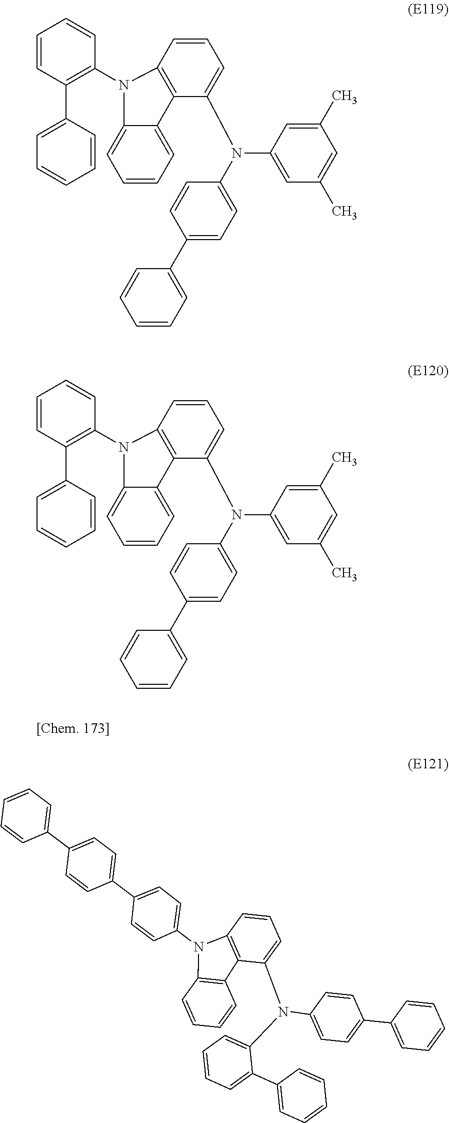 [SMILES: Cc1cc(C)cc(N(c2ccc(-c3ccccc3)cc2)c2cccc3c2c2ccccc2n3-c2ccccc2-c2ccccc2)c1 ; Cc1cc(C)cc(N(c2ccc(-c3ccccc3)cc2)c2cccc3c2c2ccccc2n3-c2ccccc2-c2ccccc2)c1 ; c1ccc(-c2ccc(-c3ccc(-n4c5ccccc5c5c(N(c6ccc(-c7ccccc7)cc6)c6ccccc6-c6ccccc6)cccc54)cc3)cc2)cc1]

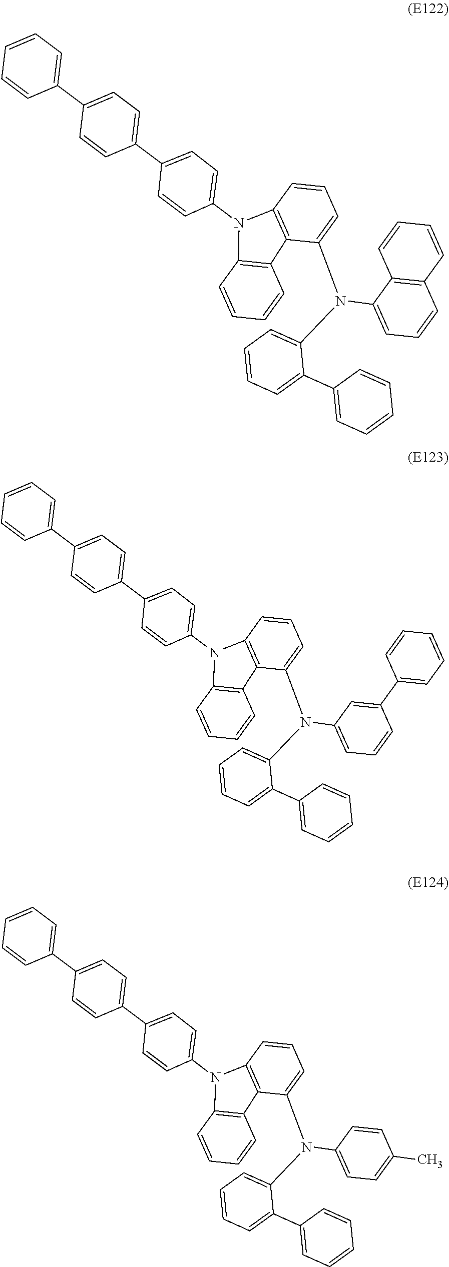 [SMILES: c1ccc(-c2ccc(-c3ccc(-n4c5ccccc5c5c(N(c6ccccc6-c6ccccc6)c6cccc7ccccc67)cccc54)cc3)cc2)cc1 ; c1ccc(-c2ccc(-c3ccc(-n4c5ccccc5c5c(N(c6cccc(-c7ccccc7)c6)c6ccccc6-c6ccccc6)cccc54)cc3)cc2)cc1 ; Cc1ccc(N(c2ccccc2-c2ccccc2)c2cccc3c2c2ccccc2n3-c2ccc(-c3ccc(-c4ccccc4)cc3)cc2)cc1]

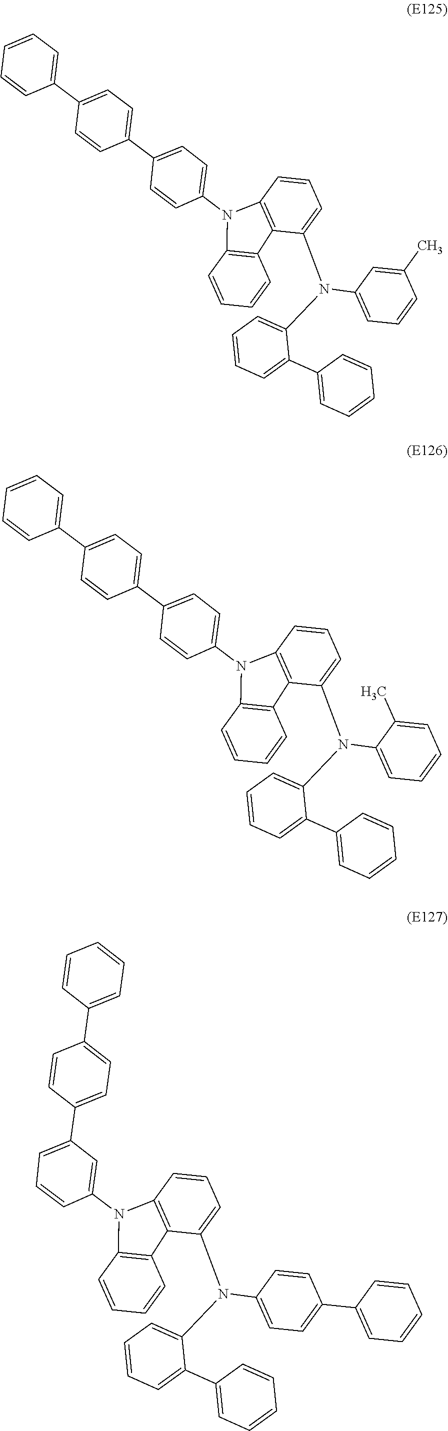 [SMILES: Cc1cccc(N(c2ccccc2-c2ccccc2)c2cccc3c2c2ccccc2n3-c2ccc(-c3ccc(-c4ccccc4)cc3)cc2)c1 ; Cc1ccccc1N(c1ccccc1-c1ccccc1)c1cccc2c1c1ccccc1n2-c1ccc(-c2ccc(-c3ccccc3)cc2)cc1 ; c1ccc(-c2ccc(-c3cccc(-n4c5ccccc5c5c(N(c6ccc(-c7ccccc7)cc6)c6ccccc6-c6ccccc6)cccc54)c3)cc2)cc1]

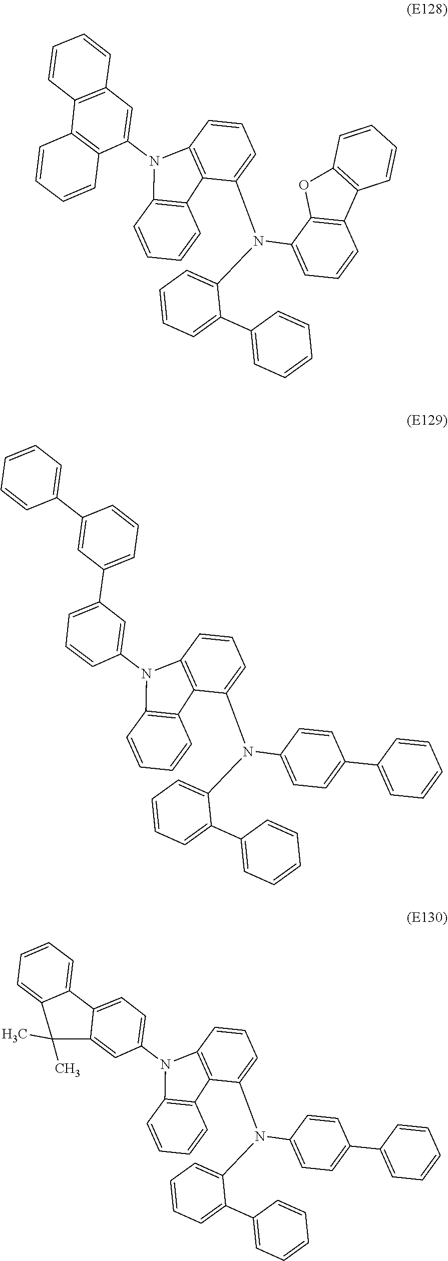 [SMILES: c1ccc(-c2ccccc2N(c2cccc3c2oc2ccccc23)c2cccc3c2c2ccccc2n3-c2cc3ccccc3c3ccccc23)cc1 ; c1ccc(-c2ccc(N(c3ccccc3-c3ccccc3)c3cccc4c3c3ccccc3n4-c3cccc(-c4cccc(-c5ccccc5)c4)c3)cc2)cc1 ; CC1(C)c2ccccc2-c2ccc(-n3c4ccccc4c4c(N(c5ccc(-c6ccccc6)cc5)c5ccccc5-c5ccccc5)cccc43)cc21]

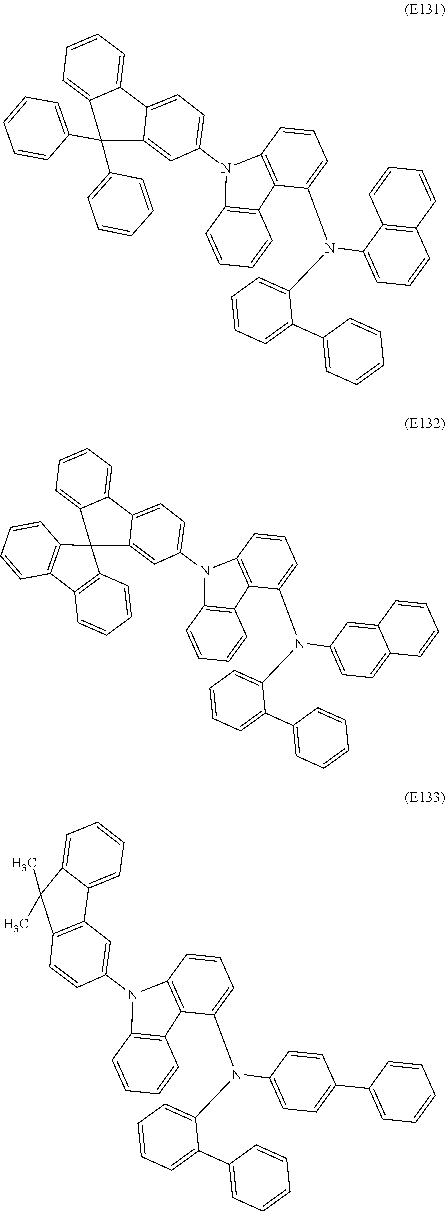 [SMILES: c1ccc(-c2ccccc2N(c2cccc3ccccc23)c2cccc3c2c2ccccc2n3-c2ccc3c(c2)C(c2ccccc2)(c2ccccc2)c2ccccc2-3)cc1 ; c1ccc(-c2ccccc2N(c2ccc3ccccc3c2)c2cccc3c2c2ccccc2n3-c2ccc3c(c2)C2(c4ccccc4-c4ccccc42)c2ccccc2-3)cc1 ; CC1(C)c2ccccc2-c2cc(-n3c4ccccc4c4c(N(c5ccc(-c6ccccc6)cc5)c5ccccc5-c5ccccc5)cccc43)ccc21]

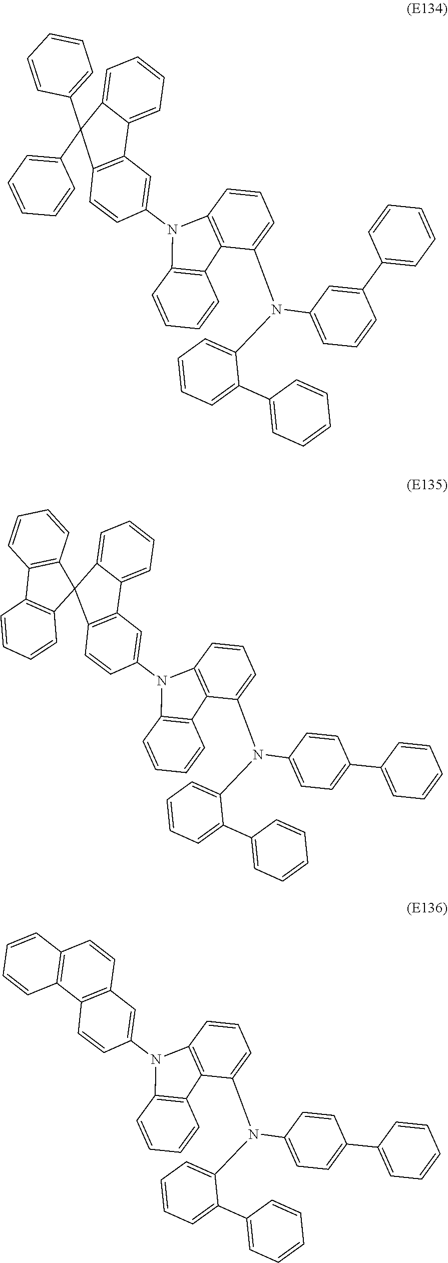 [SMILES: c1ccc(-c2cccc(N(c3ccccc3-c3ccccc3)c3cccc4c3c3ccccc3n4-c3ccc4c(c3)-c3ccccc3C4(c3ccccc3)c3ccccc3)c2)cc1 ; c1ccc(-c2ccc(N(c3ccccc3-c3ccccc3)c3cccc4c3c3ccccc3n4-c3ccc4c(c3)-c3ccccc3C43c4ccccc4-c4ccccc43)cc2)cc1 ; c1ccc(-c2ccc(N(c3ccccc3-c3ccccc3)c3cccc4c3c3ccccc3n4-c3ccc4c(ccc5ccccc54)c3)cc2)cc1]

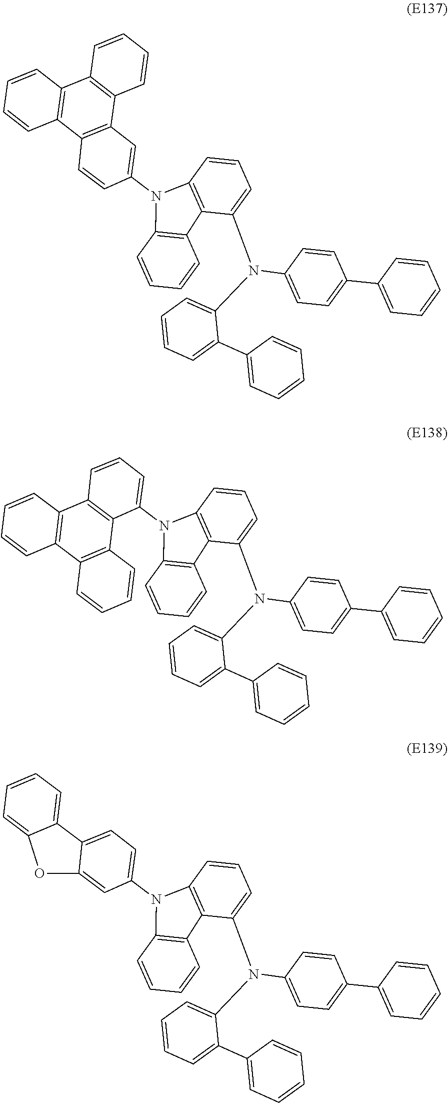 [SMILES: c1ccc(-c2ccc(N(c3ccccc3-c3ccccc3)c3cccc4c3c3ccccc3n4-c3ccc4c5ccccc5c5ccccc5c4c3)cc2)cc1 ; c1ccc(-c2ccc(N(c3ccccc3-c3ccccc3)c3cccc4c3c3ccccc3n4-c3cccc4c5ccccc5c5ccccc5c34)cc2)cc1 ; c1ccc(-c2ccc(N(c3ccccc3-c3ccccc3)c3cccc4c3c3ccccc3n4-c3ccc4c(c3)oc3ccccc34)cc2)cc1]

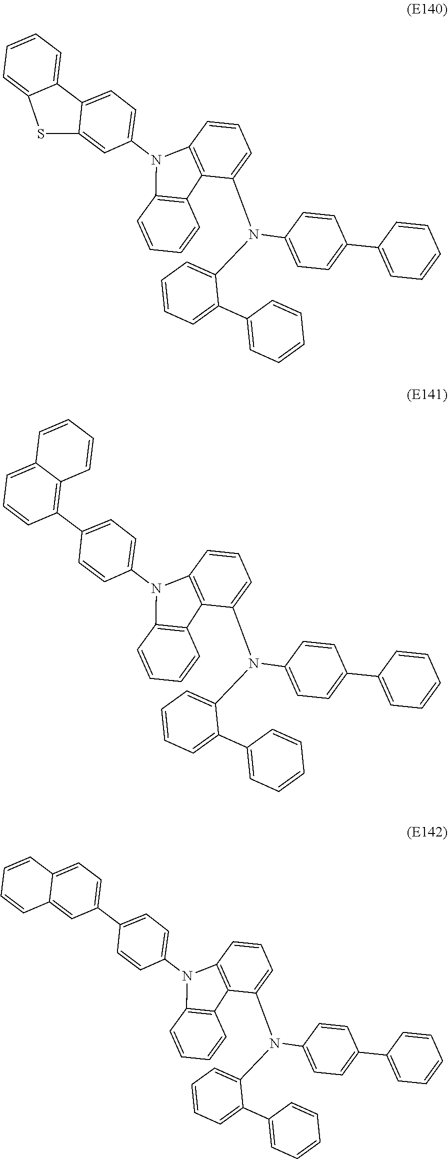 [SMILES: c1ccc(-c2ccc(N(c3ccccc3-c3ccccc3)c3cccc4c3c3ccccc3n4-c3ccc4c(c3)sc3ccccc34)cc2)cc1 ; c1ccc(-c2ccc(N(c3ccccc3-c3ccccc3)c3cccc4c3c3ccccc3n4-c3ccc(-c4cccc5ccccc45)cc3)cc2)cc1 ; c1ccc(-c2ccc(N(c3ccccc3-c3ccccc3)c3cccc4c3c3ccccc3n4-c3ccc(-c4ccc5ccccc5c4)cc3)cc2)cc1]

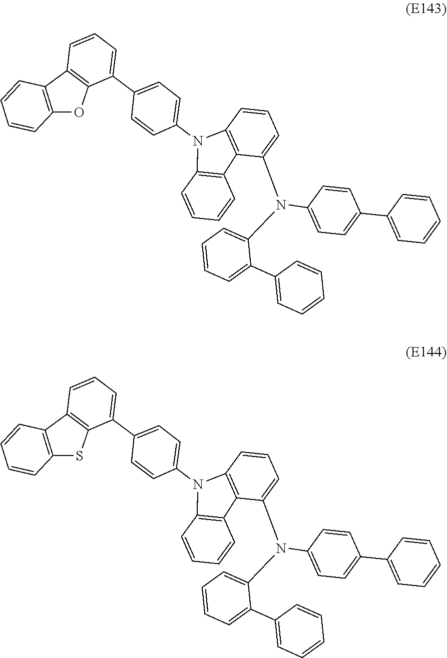 [SMILES: c1ccc(-c2ccc(N(c3ccccc3-c3ccccc3)c3cccc4c3c3ccccc3n4-c3ccc(-c4cccc5c4oc4ccccc45)cc3)cc2)cc1 ; c1ccc(-c2ccc(N(c3ccccc3-c3ccccc3)c3cccc4c3c3ccccc3n4-c3ccc(-c4cccc5c4sc4ccccc45)cc3)cc2)cc1]

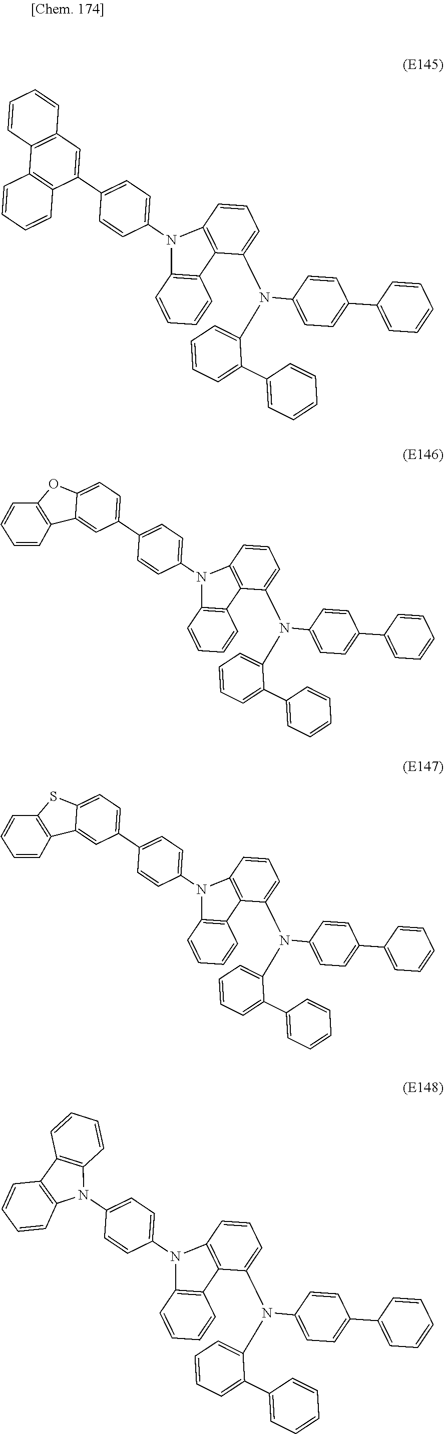 [SMILES: c1ccc(-c2ccc(N(c3ccccc3-c3ccccc3)c3cccc4c3c3ccccc3n4-c3ccc(-c4cc5ccccc5c5ccccc45)cc3)cc2)cc1 ; c1ccc(-c2ccc(N(c3ccccc3-c3ccccc3)c3cccc4c3c3ccccc3n4-c3ccc(-c4ccc5oc6ccccc6c5c4)cc3)cc2)cc1 ; c1ccc(-c2ccc(N(c3ccccc3-c3ccccc3)c3cccc4c3c3ccccc3n4-c3ccc(-c4ccc5sc6ccccc6c5c4)cc3)cc2)cc1 ; c1ccc(-c2ccc(N(c3ccccc3-c3ccccc3)c3cccc4c3c3ccccc3n4-c3ccc(-n4c5ccccc5c5ccccc54)cc3)cc2)cc1]

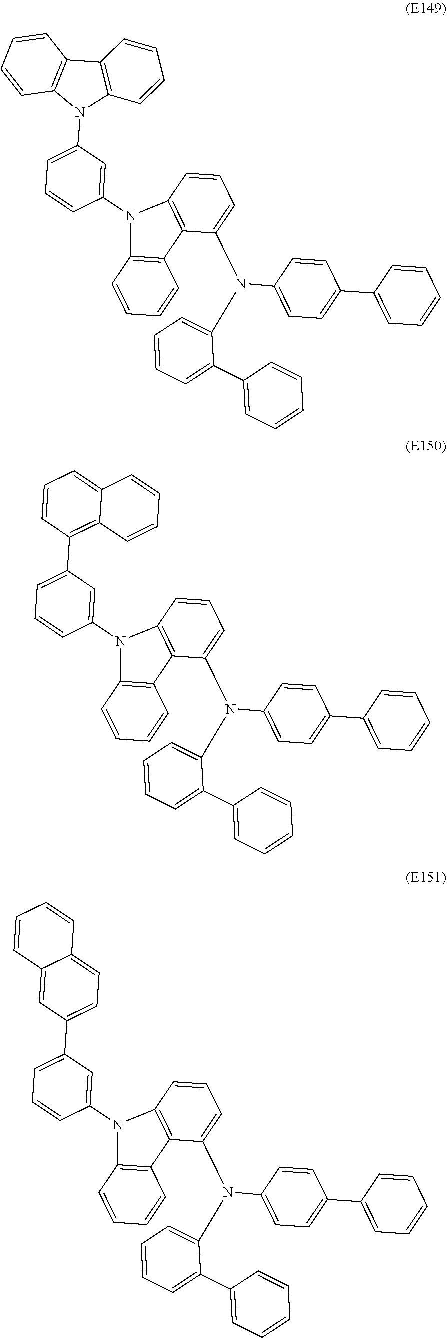 [SMILES: c1ccc(-c2ccc(N(c3ccccc3-c3ccccc3)c3cccc4c3c3ccccc3n4-c3cccc(-n4c5ccccc5c5ccccc54)c3)cc2)cc1 ; c1ccc(-c2ccc(N(c3ccccc3-c3ccccc3)c3cccc4c3c3ccccc3n4-c3cccc(-c4cccc5ccccc45)c3)cc2)cc1 ; c1ccc(-c2ccc(N(c3ccccc3-c3ccccc3)c3cccc4c3c3ccccc3n4-c3cccc(-c4ccc5ccccc5c4)c3)cc2)cc1]

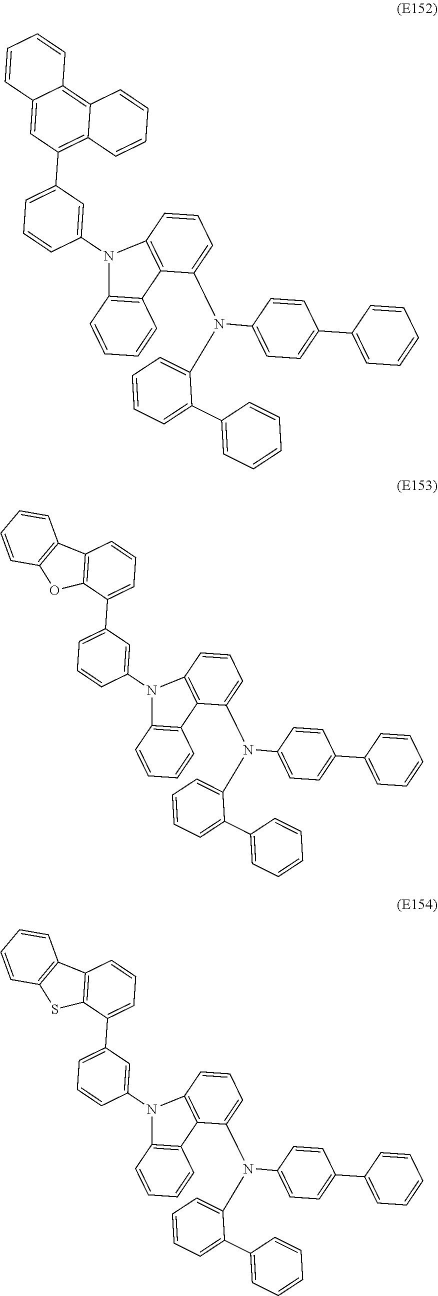 [SMILES: c1ccc(-c2ccc(N(c3ccccc3-c3ccccc3)c3cccc4c3c3ccccc3n4-c3cccc(-c4cc5ccccc5c5ccccc45)c3)cc2)cc1 ; c1ccc(-c2ccc(N(c3ccccc3-c3ccccc3)c3cccc4c3c3ccccc3n4-c3cccc(-c4cccc5c4oc4ccccc45)c3)cc2)cc1 ; c1ccc(-c2ccc(N(c3ccccc3-c3ccccc3)c3cccc4c3c3ccccc3n4-c3cccc(-c4cccc5c4sc4ccccc45)c3)cc2)cc1]

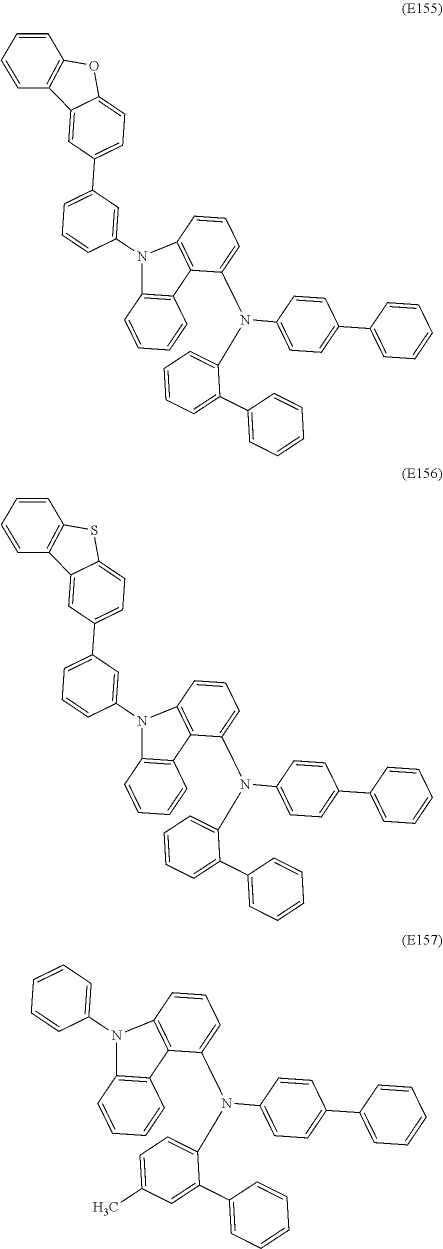 [SMILES: c1ccc(-c2ccc(N(c3ccccc3-c3ccccc3)c3cccc4c3c3ccccc3n4-c3cccc(-c4ccc5oc6ccccc6c5c4)c3)cc2)cc1 ; c1ccc(-c2ccc(N(c3ccccc3-c3ccccc3)c3cccc4c3c3ccccc3n4-c3cccc(-c4ccc5sc6ccccc6c5c4)c3)cc2)cc1 ; Cc1ccc(N(c2ccc(-c3ccccc3)cc2)c2cccc3c2c2ccccc2n3-c2ccccc2)c(-c2ccccc2)c1]

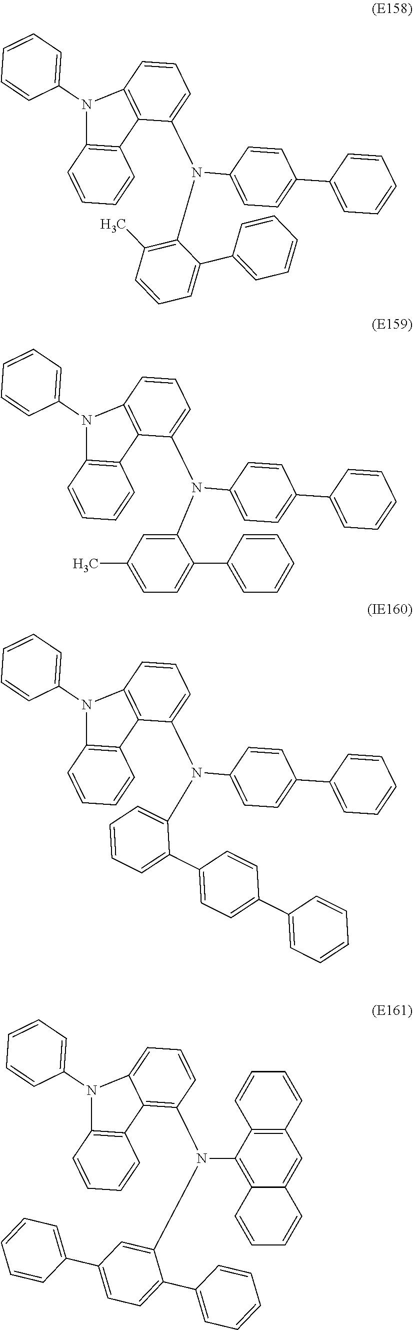 [SMILES: Cc1cccc(-c2ccccc2)c1N(c1ccc(-c2ccccc2)cc1)c1cccc2c1c1ccccc1n2-c1ccccc1 ; Cc1ccc(-c2ccccc2)c(N(c2ccc(-c3ccccc3)cc2)c2cccc3c2c2ccccc2n3-c2ccccc2)c1 ; c1ccc(-c2ccc(-c3ccccc3N(c3ccc(-c4ccccc4)cc3)c3cccc4c3c3ccccc3n4-c3ccccc3)cc2)cc1 ; c1ccc(-c2ccc(-c3ccccc3)c(N(c3c4ccccc4cc4ccccc34)c3cccc4c3c3ccccc3n4-c3ccccc3)c2)cc1]

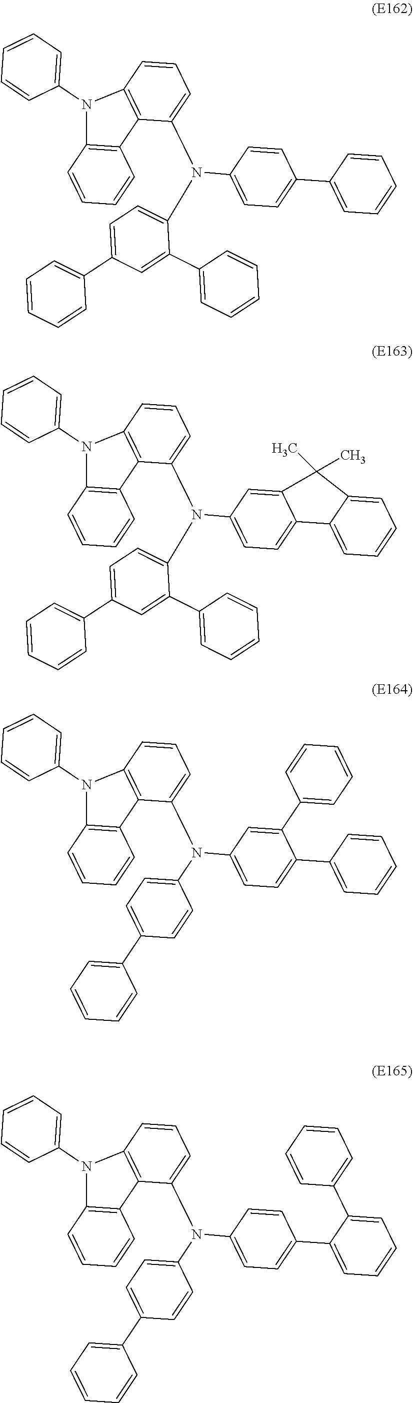 [SMILES: c1ccc(-c2ccc(N(c3ccc(-c4ccccc4)cc3-c3ccccc3)c3cccc4c3c3ccccc3n4-c3ccccc3)cc2)cc1 ; CC1(C)c2ccccc2-c2ccc(N(c3ccc(-c4ccccc4)cc3-c3ccccc3)c3cccc4c3c3ccccc3n4-c3ccccc3)cc21 ; c1ccc(-c2ccc(N(c3ccc(-c4ccccc4)c(-c4ccccc4)c3)c3cccc4c3c3ccccc3n4-c3ccccc3)cc2)cc1 ; c1ccc(-c2ccc(N(c3ccc(-c4ccccc4-c4ccccc4)cc3)c3cccc4c3c3ccccc3n4-c3ccccc3)cc2)cc1]

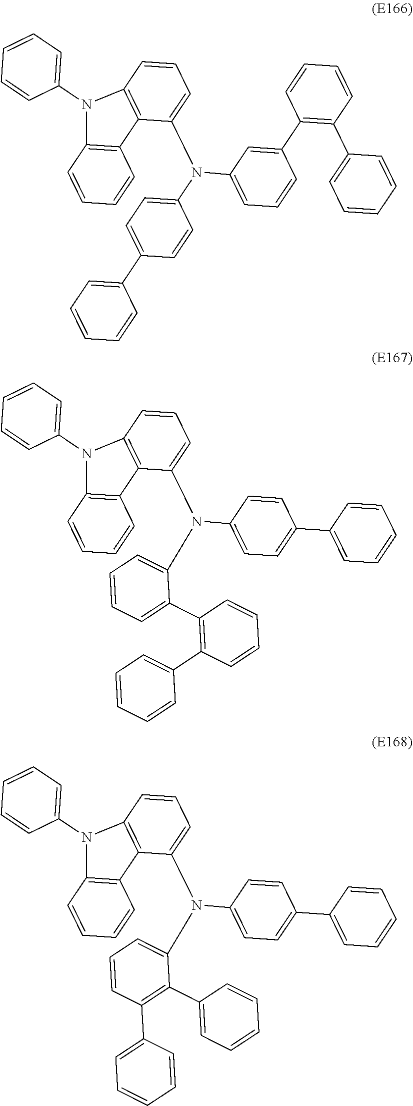 [SMILES: c1ccc(-c2ccc(N(c3cccc(-c4ccccc4-c4ccccc4)c3)c3cccc4c3c3ccccc3n4-c3ccccc3)cc2)cc1 ; c1ccc(-c2ccc(N(c3ccccc3-c3ccccc3-c3ccccc3)c3cccc4c3c3ccccc3n4-c3ccccc3)cc2)cc1 ; c1ccc(-c2ccc(N(c3cccc(-c4ccccc4)c3-c3ccccc3)c3cccc4c3c3ccccc3n4-c3ccccc3)cc2)cc1]

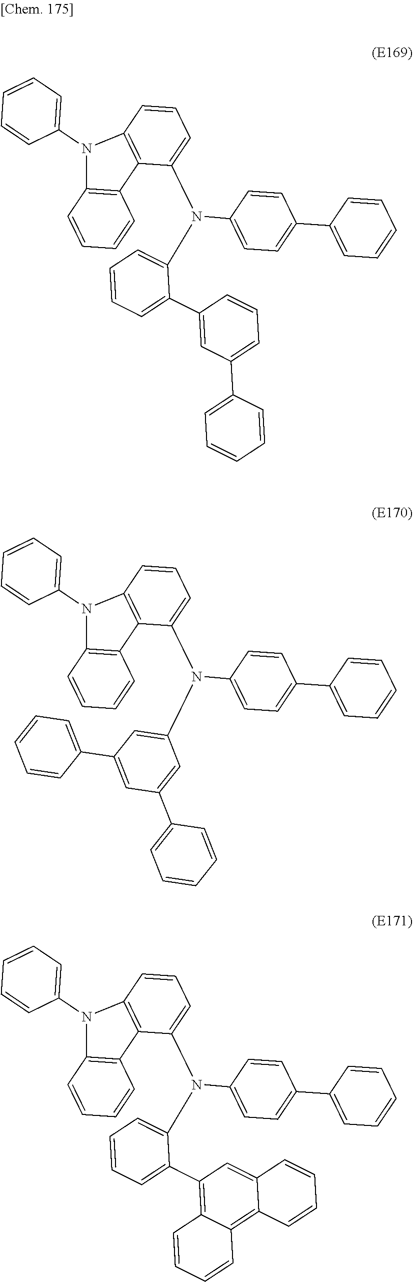 [SMILES: c1ccc(-c2ccc(N(c3ccccc3-c3cccc(-c4ccccc4)c3)c3cccc4c3c3ccccc3n4-c3ccccc3)cc2)cc1 ; c1ccc(-c2ccc(N(c3cc(-c4ccccc4)cc(-c4ccccc4)c3)c3cccc4c3c3ccccc3n4-c3ccccc3)cc2)cc1 ; c1ccc(-c2ccc(N(c3ccccc3-c3cc4ccccc4c4ccccc34)c3cccc4c3c3ccccc3n4-c3ccccc3)cc2)cc1]

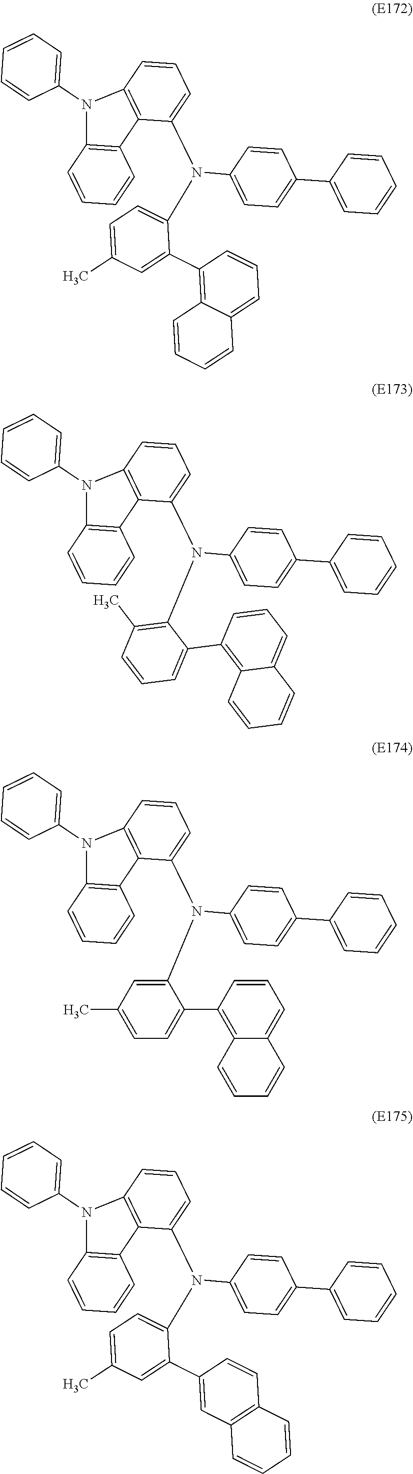 [SMILES: Cc1ccc(N(c2ccc(-c3ccccc3)cc2)c2cccc3c2c2ccccc2n3-c2ccccc2)c(-c2cccc3ccccc23)c1 ; Cc1cccc(-c2cccc3ccccc23)c1N(c1ccc(-c2ccccc2)cc1)c1cccc2c1c1ccccc1n2-c1ccccc1 ; Cc1ccc(-c2cccc3ccccc23)c(N(c2ccc(-c3ccccc3)cc2)c2cccc3c2c2ccccc2n3-c2ccccc2)c1 ; Cc1ccc(N(c2ccc(-c3ccccc3)cc2)c2cccc3c2c2ccccc2n3-c2ccccc2)c(-c2ccc3ccccc3c2)c1]

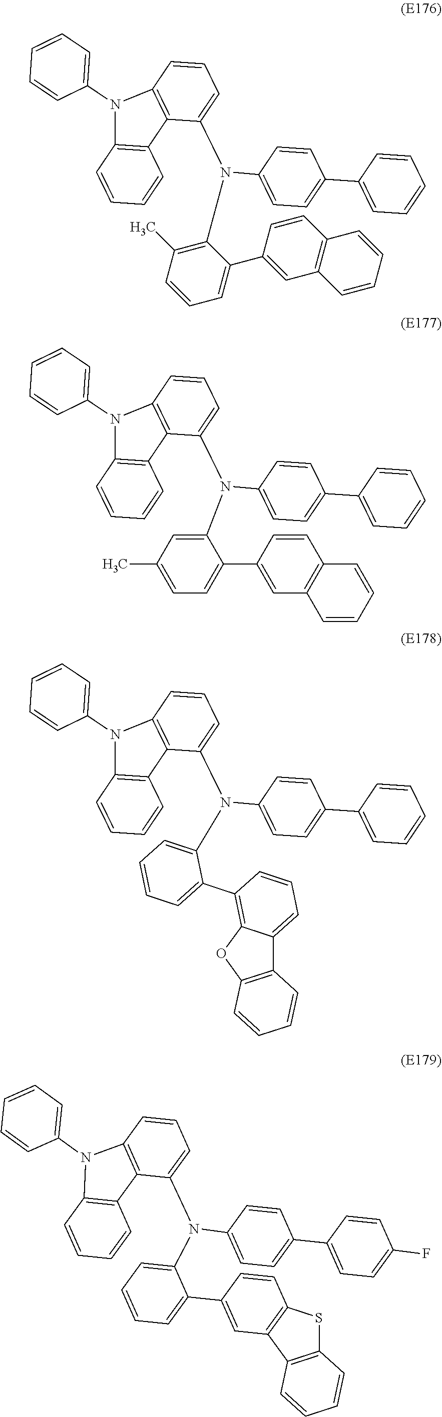 [SMILES: Cc1cccc(-c2ccc3ccccc3c2)c1N(c1ccc(-c2ccccc2)cc1)c1cccc2c1c1ccccc1n2-c1ccccc1 ; Cc1ccc(-c2ccc3ccccc3c2)c(N(c2ccc(-c3ccccc3)cc2)c2cccc3c2c2ccccc2n3-c2ccccc2)c1 ; c1ccc(-c2ccc(N(c3ccccc3-c3cccc4c3oc3ccccc34)c3cccc4c3c3ccccc3n4-c3ccccc3)cc2)cc1 ; Fc1ccc(-c2ccc(N(c3ccccc3-c3ccc4sc5ccccc5c4c3)c3cccc4c3c3ccccc3n4-c3ccccc3)cc2)cc1]

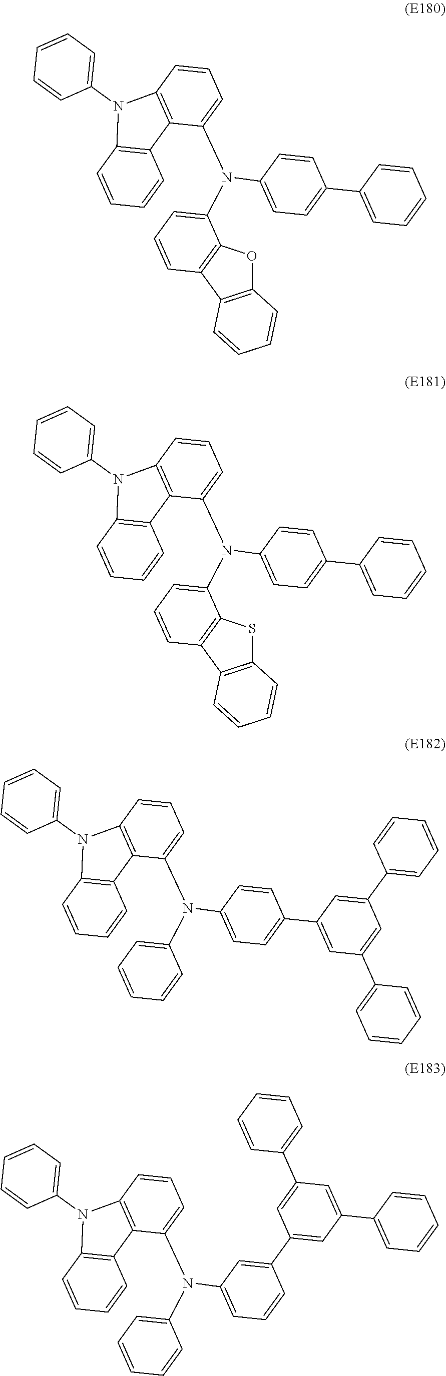 [SMILES: c1ccc(-c2ccc(N(c3cccc4c3oc3ccccc34)c3cccc4c3c3ccccc3n4-c3ccccc3)cc2)cc1 ; c1ccc(-c2ccc(N(c3cccc4c3sc3ccccc34)c3cccc4c3c3ccccc3n4-c3ccccc3)cc2)cc1 ; c1ccc(-c2cc(-c3ccccc3)cc(-c3ccc(N(c4ccccc4)c4cccc5c4c4ccccc4n5-c4ccccc4)cc3)c2)cc1 ; c1ccc(-c2cc(-c3ccccc3)cc(-c3cccc(N(c4ccccc4)c4cccc5c4c4ccccc4n5-c4ccccc4)c3)c2)cc1]

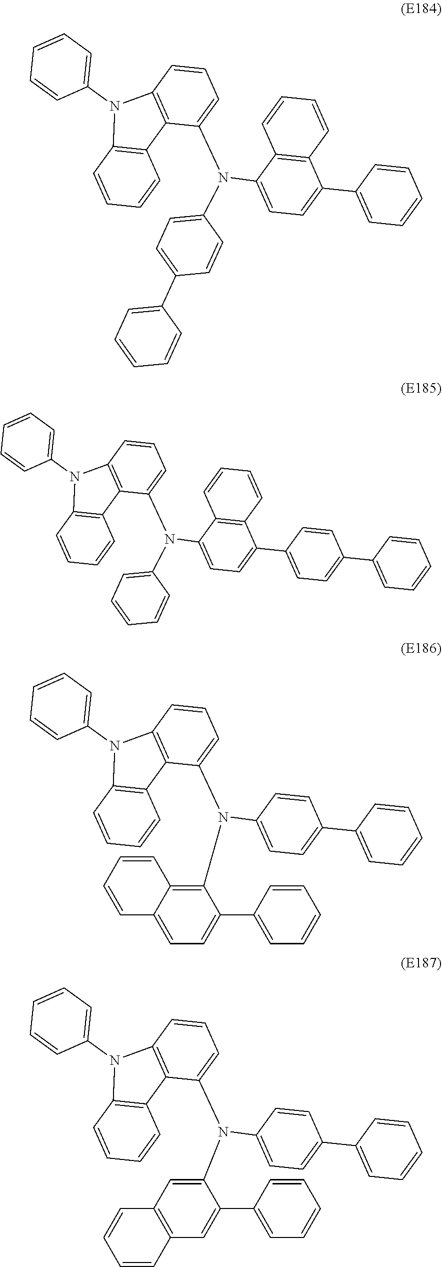 [SMILES: c1ccc(-c2ccc(N(c3ccc(-c4ccccc4)c4ccccc34)c3cccc4c3c3ccccc3n4-c3ccccc3)cc2)cc1 ; c1ccc(-c2ccc(-c3ccc(N(c4ccccc4)c4cccc5c4c4ccccc4n5-c4ccccc4)c4ccccc34)cc2)cc1 ; c1ccc(-c2ccc(N(c3c(-c4ccccc4)ccc4ccccc34)c3cccc4c3c3ccccc3n4-c3ccccc3)cc2)cc1 ; c1ccc(-c2ccc(N(c3cc4ccccc4cc3-c3ccccc3)c3cccc4c3c3ccccc3n4-c3ccccc3)cc2)cc1]

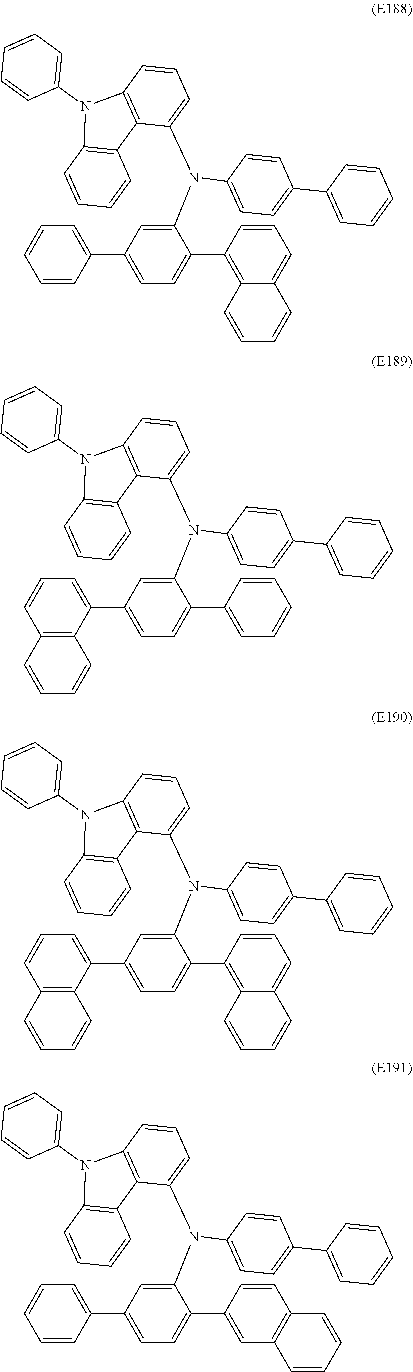

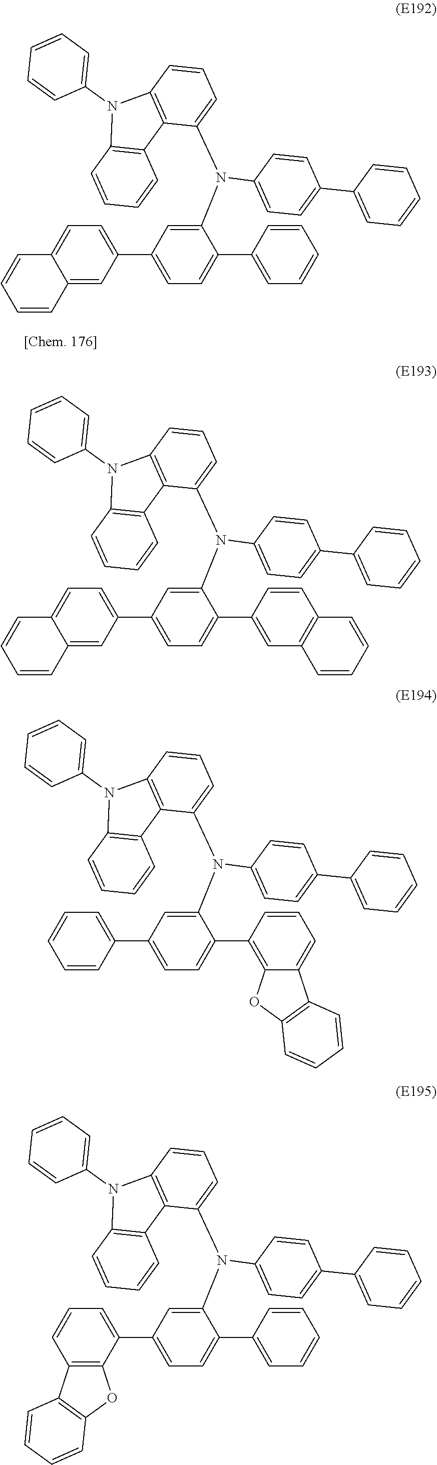

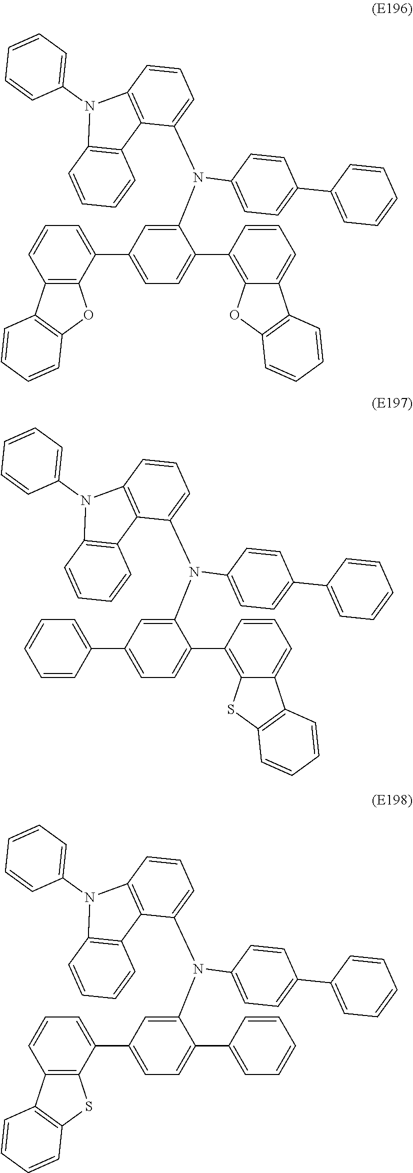 [SMILES: c1ccc(-c2ccc(N(c3cc(-c4cccc5c4oc4ccccc45)ccc3-c3cccc4c3oc3ccccc34)c3cccc4c3c3ccccc3n4-c3ccccc3)cc2)cc1 ; c1ccc(-c2ccc(N(c3cc(-c4ccccc4)ccc3-c3cccc4c3sc3ccccc34)c3cccc4c3c3ccccc3n4-c3ccccc3)cc2)cc1 ; c1ccc(-c2ccc(N(c3cc(-c4cccc5c4sc4ccccc45)ccc3-c3ccccc3)c3cccc4c3c3ccccc3n4-c3ccccc3)cc2)cc1]

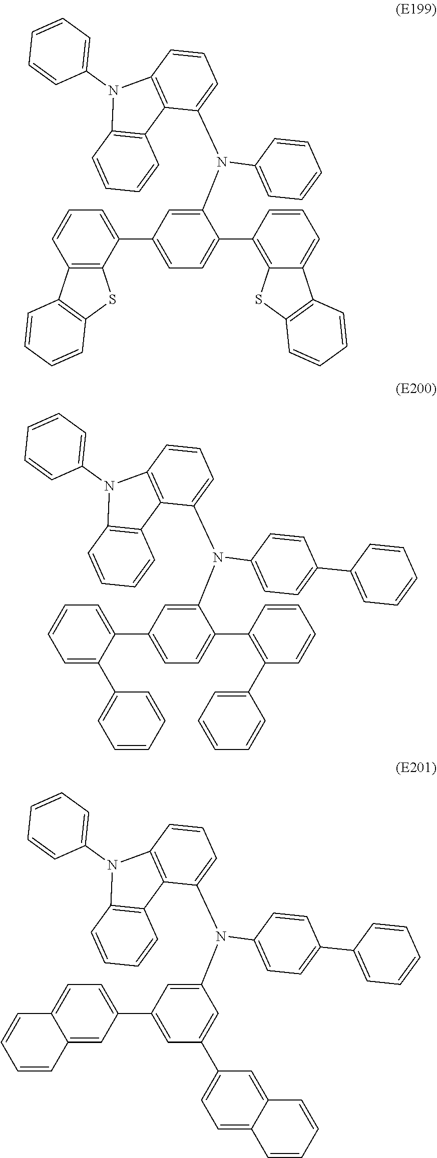 [SMILES: c1ccc(N(c2cc(-c3cccc4c3sc3ccccc34)ccc2-c2cccc3c2sc2ccccc23)c2cccc3c2c2ccccc2n3-c2ccccc2)cc1 ; c1ccc(-c2ccc(N(c3cc(-c4ccccc4-c4ccccc4)ccc3-c3ccccc3-c3ccccc3)c3cccc4c3c3ccccc3n4-c3ccccc3)cc2)cc1 ; c1ccc(-c2ccc(N(c3cc(-c4ccc5ccccc5c4)cc(-c4ccc5ccccc5c4)c3)c3cccc4c3c3ccccc3n4-c3ccccc3)cc2)cc1]

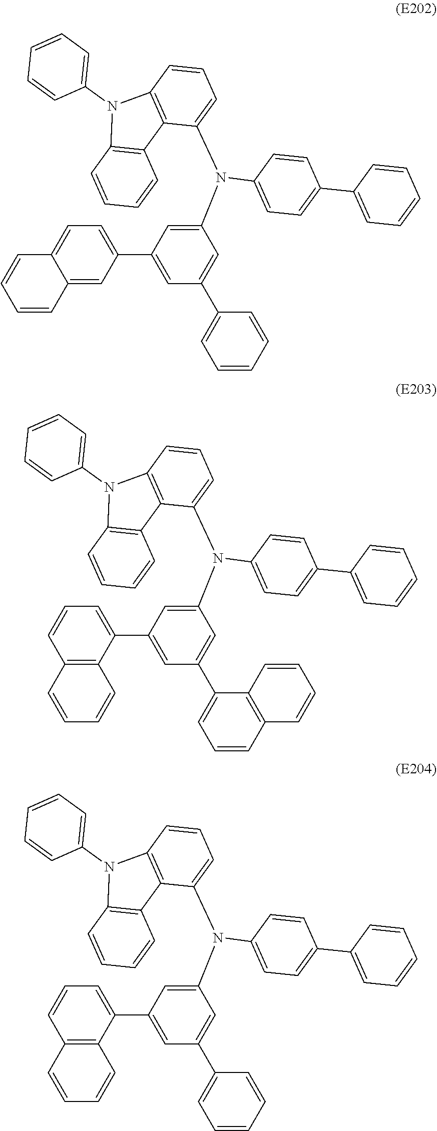 [SMILES: c1ccc(-c2ccc(N(c3cc(-c4ccccc4)cc(-c4ccc5ccccc5c4)c3)c3cccc4c3c3ccccc3n4-c3ccccc3)cc2)cc1 ; c1ccc(-c2ccc(N(c3cc(-c4cccc5ccccc45)cc(-c4cccc5ccccc45)c3)c3cccc4c3c3ccccc3n4-c3ccccc3)cc2)cc1 ; c1ccc(-c2ccc(N(c3cc(-c4ccccc4)cc(-c4cccc5ccccc45)c3)c3cccc4c3c3ccccc3n4-c3ccccc3)cc2)cc1]

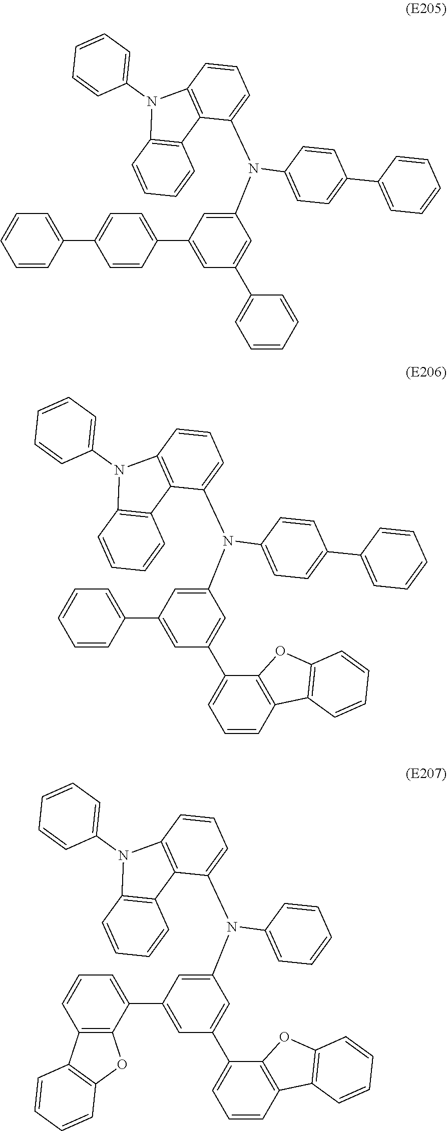 [SMILES: c1ccc(-c2ccc(-c3cc(-c4ccccc4)cc(N(c4ccc(-c5ccccc5)cc4)c4cccc5c4c4ccccc4n5-c4ccccc4)c3)cc2)cc1 ; c1ccc(-c2ccc(N(c3cc(-c4ccccc4)cc(-c4cccc5c4oc4ccccc45)c3)c3cccc4c3c3ccccc3n4-c3ccccc3)cc2)cc1 ; c1ccc(N(c2cc(-c3cccc4c3oc3ccccc34)cc(-c3cccc4c3oc3ccccc34)c2)c2cccc3c2c2ccccc2n3-c2ccccc2)cc1]

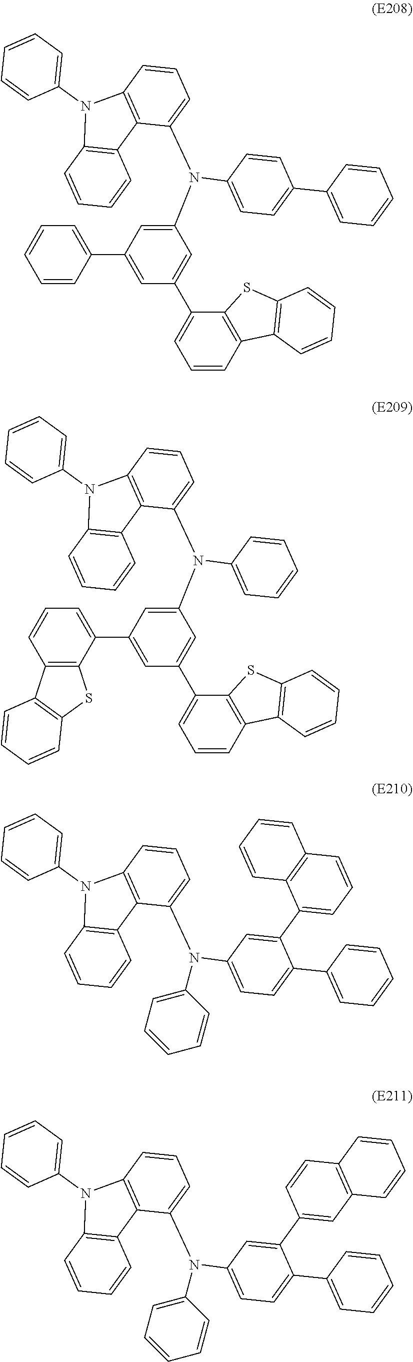 [SMILES: c1ccc(-c2ccc(N(c3cc(-c4ccccc4)cc(-c4cccc5c4sc4ccccc45)c3)c3cccc4c3c3ccccc3n4-c3ccccc3)cc2)cc1 ; c1ccc(N(c2cc(-c3cccc4c3sc3ccccc34)cc(-c3cccc4c3sc3ccccc34)c2)c2cccc3c2c2ccccc2n3-c2ccccc2)cc1 ; c1ccc(-c2ccc(N(c3ccccc3)c3cccc4c3c3ccccc3n4-c3ccccc3)cc2-c2cccc3ccccc23)cc1 ; c1ccc(-c2ccc(N(c3ccccc3)c3cccc4c3c3ccccc3n4-c3ccccc3)cc2-c2ccc3ccccc3c2)cc1]

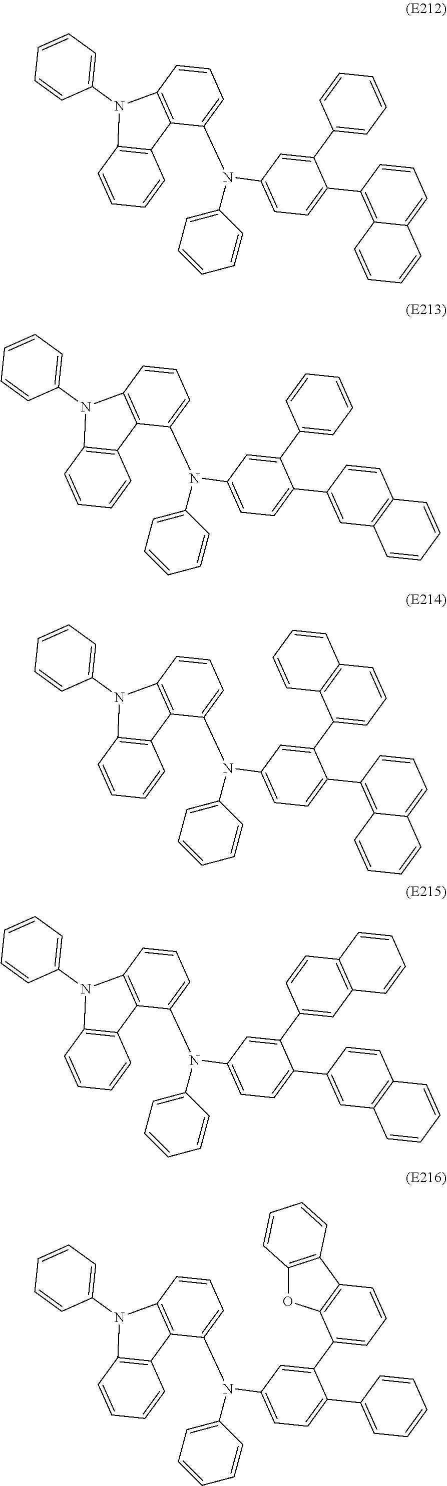 [SMILES: c1ccc(-c2cc(N(c3ccccc3)c3cccc4c3c3ccccc3n4-c3ccccc3)ccc2-c2cccc3ccccc23)cc1 ; c1ccc(-c2cc(N(c3ccccc3)c3cccc4c3c3ccccc3n4-c3ccccc3)ccc2-c2ccc3ccccc3c2)cc1 ; c1ccc(N(c2ccc(-c3cccc4ccccc34)c(-c3cccc4ccccc34)c2)c2cccc3c2c2ccccc2n3-c2ccccc2)cc1 ; c1ccc(N(c2ccc(-c3ccc4ccccc4c3)c(-c3ccc4ccccc4c3)c2)c2cccc3c2c2ccccc2n3-c2ccccc2)cc1 ; c1ccc(-c2ccc(N(c3ccccc3)c3cccc4c3c3ccccc3n4-c3ccccc3)cc2-c2cccc3c2oc2ccccc23)cc1]

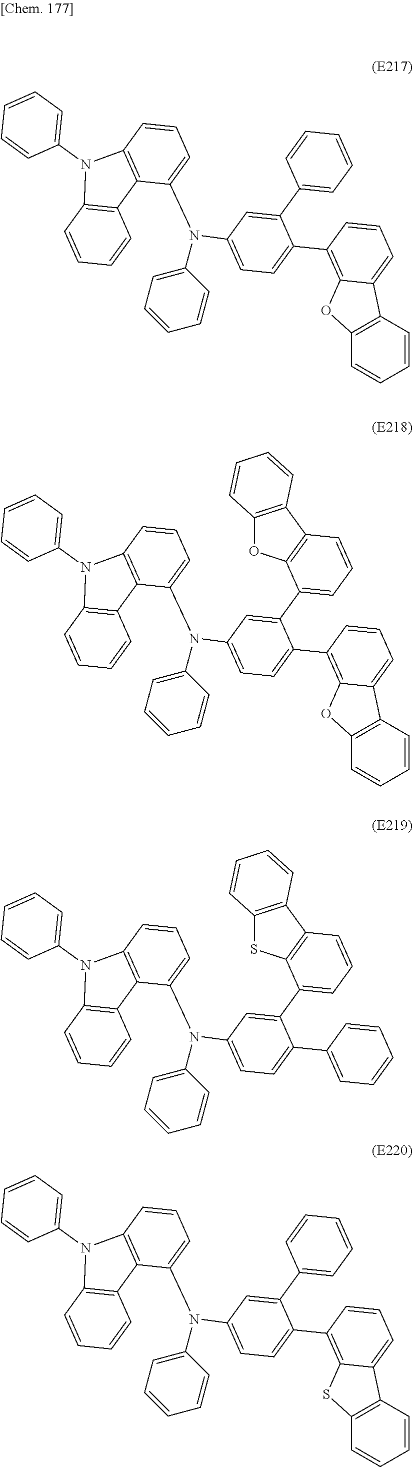 [SMILES: c1ccc(-c2cc(N(c3ccccc3)c3cccc4c3c3ccccc3n4-c3ccccc3)ccc2-c2cccc3c2oc2ccccc23)cc1 ; c1ccc(N(c2ccc(-c3cccc4c3oc3ccccc34)c(-c3cccc4c3oc3ccccc34)c2)c2cccc3c2c2ccccc2n3-c2ccccc2)cc1 ; c1ccc(-c2ccc(N(c3ccccc3)c3cccc4c3c3ccccc3n4-c3ccccc3)cc2-c2cccc3c2sc2ccccc23)cc1 ; c1ccc(-c2cc(N(c3ccccc3)c3cccc4c3c3ccccc3n4-c3ccccc3)ccc2-c2cccc3c2sc2ccccc23)cc1]

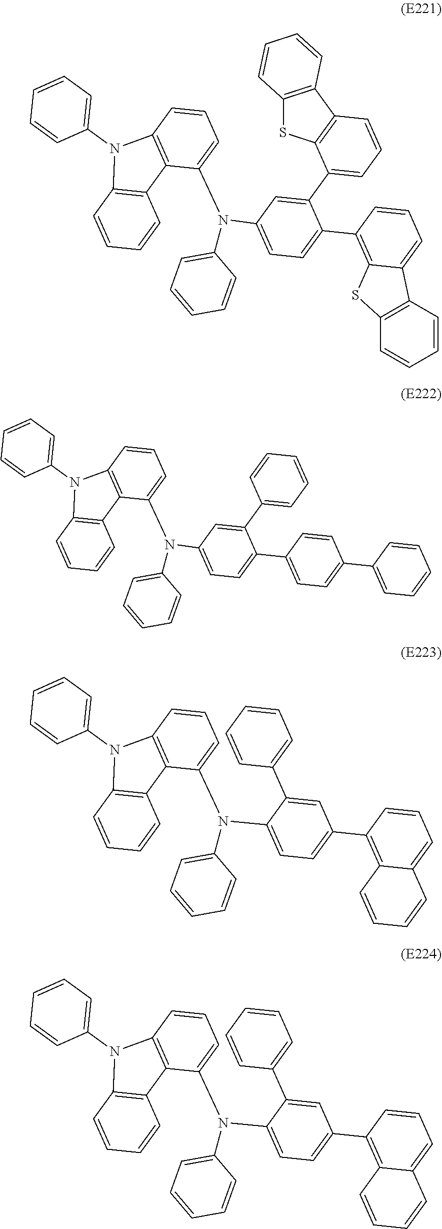 [SMILES: c1ccc(N(c2ccc(-c3cccc4c3sc3ccccc34)c(-c3cccc4c3sc3ccccc34)c2)c2cccc3c2c2ccccc2n3-c2ccccc2)cc1 ; c1ccc(-c2ccc(-c3ccc(N(c4ccccc4)c4cccc5c4c4ccccc4n5-c4ccccc4)cc3-c3ccccc3)cc2)cc1 ; c1ccc(-c2cc(-c3cccc4ccccc34)ccc2N(c2ccccc2)c2cccc3c2c2ccccc2n3-c2ccccc2)cc1 ; c1ccc(-c2cc(-c3cccc4ccccc34)ccc2N(c2ccccc2)c2cccc3c2c2ccccc2n3-c2ccccc2)cc1]

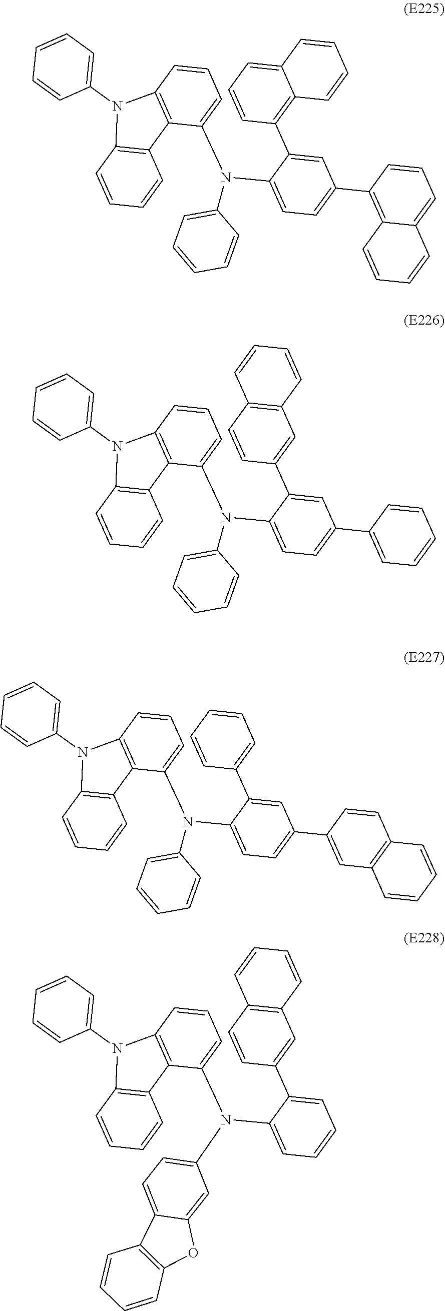 [SMILES: c1ccc(N(c2ccc(-c3cccc4ccccc34)cc2-c2cccc3ccccc23)c2cccc3c2c2ccccc2n3-c2ccccc2)cc1 ; c1ccc(-c2ccc(N(c3ccccc3)c3cccc4c3c3ccccc3n4-c3ccccc3)c(-c3ccc4ccccc4c3)c2)cc1 ; c1ccc(-c2cc(-c3ccc4ccccc4c3)ccc2N(c2ccccc2)c2cccc3c2c2ccccc2n3-c2ccccc2)cc1 ; c1ccc(-n2c3ccccc3c3c(N(c4ccc5c(c4)oc4ccccc45)c4ccccc4-c4ccc5ccccc5c4)cccc32)cc1]

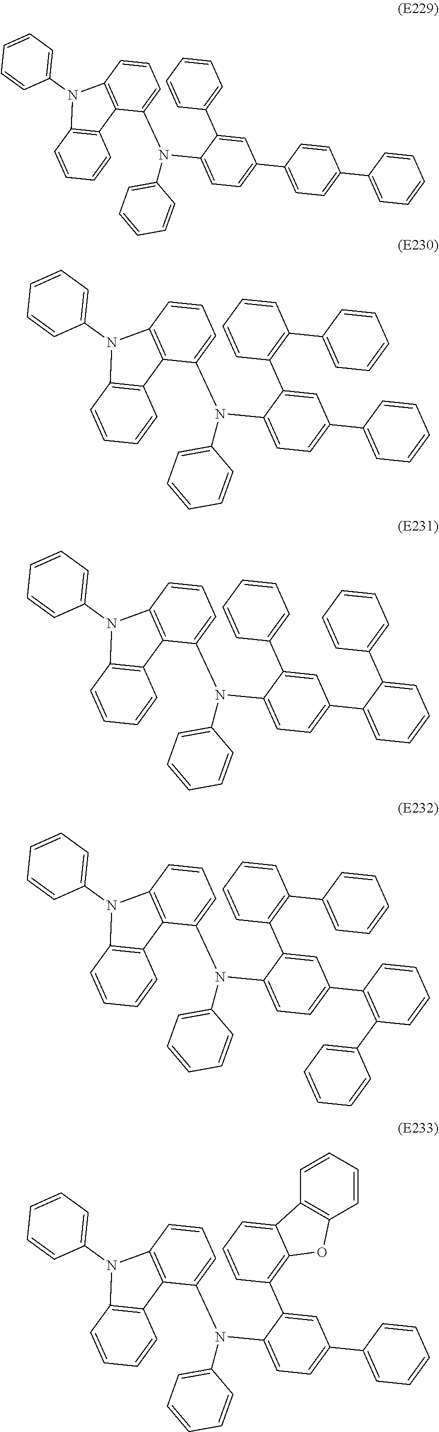 [SMILES: c1ccc(-c2ccc(-c3ccc(N(c4ccccc4)c4cccc5c4c4ccccc4n5-c4ccccc4)c(-c4ccccc4)c3)cc2)cc1 ; c1ccc(-c2ccc(N(c3ccccc3)c3cccc4c3c3ccccc3n4-c3ccccc3)c(-c3ccccc3-c3ccccc3)c2)cc1 ; c1ccc(-c2ccccc2-c2ccc(N(c3ccccc3)c3cccc4c3c3ccccc3n4-c3ccccc3)c(-c3ccccc3)c2)cc1 ; c1ccc(-c2ccccc2-c2ccc(N(c3ccccc3)c3cccc4c3c3ccccc3n4-c3ccccc3)c(-c3ccccc3-c3ccccc3)c2)cc1 ; c1ccc(-c2ccc(N(c3ccccc3)c3cccc4c3c3ccccc3n4-c3ccccc3)c(-c3cccc4c3oc3ccccc34)c2)cc1]

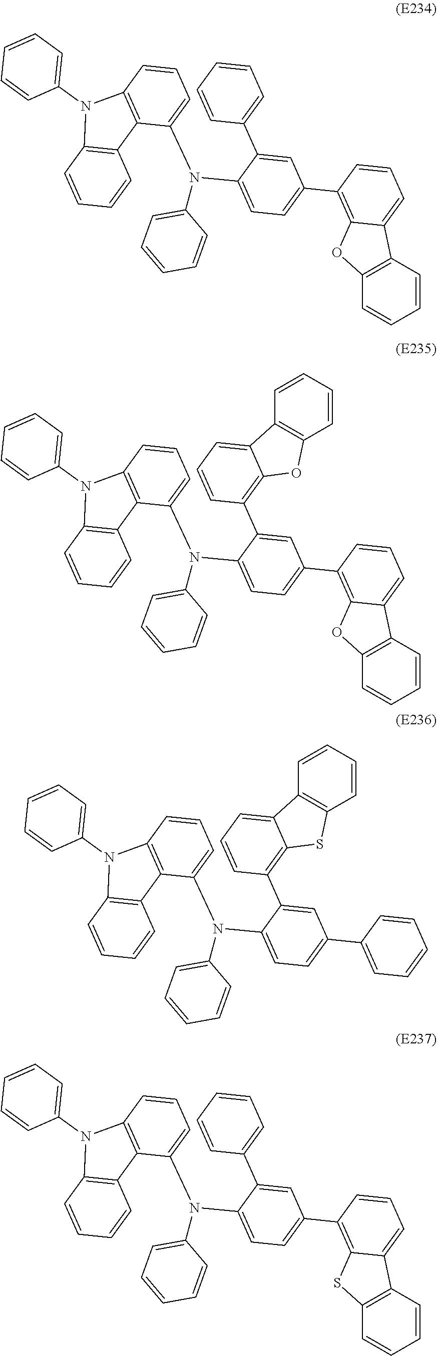 [SMILES: c1ccc(-c2cc(-c3cccc4c3oc3ccccc34)ccc2N(c2ccccc2)c2cccc3c2c2ccccc2n3-c2ccccc2)cc1 ; c1ccc(N(c2ccc(-c3cccc4c3oc3ccccc34)cc2-c2cccc3c2oc2ccccc23)c2cccc3c2c2ccccc2n3-c2ccccc2)cc1 ; c1ccc(-c2ccc(N(c3ccccc3)c3cccc4c3c3ccccc3n4-c3ccccc3)c(-c3cccc4c3sc3ccccc34)c2)cc1 ; c1ccc(-c2cc(-c3cccc4c3sc3ccccc34)ccc2N(c2ccccc2)c2cccc3c2c2ccccc2n3-c2ccccc2)cc1]

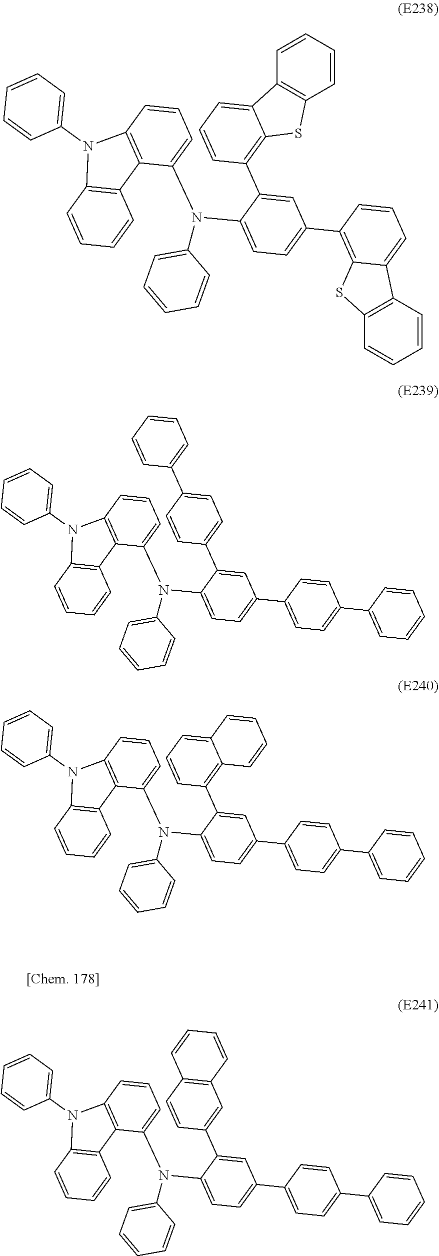 [SMILES: c1ccc(N(c2ccc(-c3cccc4c3sc3ccccc34)cc2-c2cccc3c2sc2ccccc23)c2cccc3c2c2ccccc2n3-c2ccccc2)cc1 ; c1ccc(-c2ccc(-c3ccc(N(c4ccccc4)c4cccc5c4c4ccccc4n5-c4ccccc4)c(-c4ccc(-c5ccccc5)cc4)c3)cc2)cc1 ; c1ccc(-c2ccc(-c3ccc(N(c4ccccc4)c4cccc5c4c4ccccc4n5-c4ccccc4)c(-c4cccc5ccccc45)c3)cc2)cc1 ; c1ccc(-c2ccc(-c3ccc(N(c4ccccc4)c4cccc5c4c4ccccc4n5-c4ccccc4)c(-c4ccc5ccccc5c4)c3)cc2)cc1]

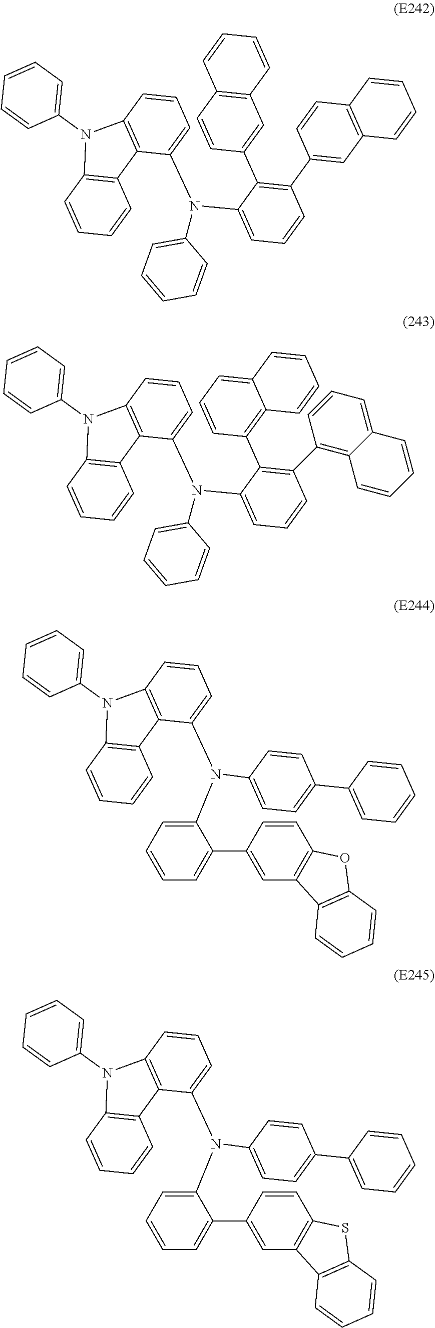 [SMILES: c1ccc(N(c2cccc(-c3ccc4ccccc4c3)c2-c2ccc3ccccc3c2)c2cccc3c2c2ccccc2n3-c2ccccc2)cc1 ; c1ccc(N(c2cccc(-c3cccc4ccccc34)c2-c2cccc3ccccc23)c2cccc3c2c2ccccc2n3-c2ccccc2)cc1 ; c1ccc(-c2ccc(N(c3ccccc3-c3ccc4oc5ccccc5c4c3)c3cccc4c3c3ccccc3n4-c3ccccc3)cc2)cc1 ; c1ccc(-c2ccc(N(c3ccccc3-c3ccc4sc5ccccc5c4c3)c3cccc4c3c3ccccc3n4-c3ccccc3)cc2)cc1]

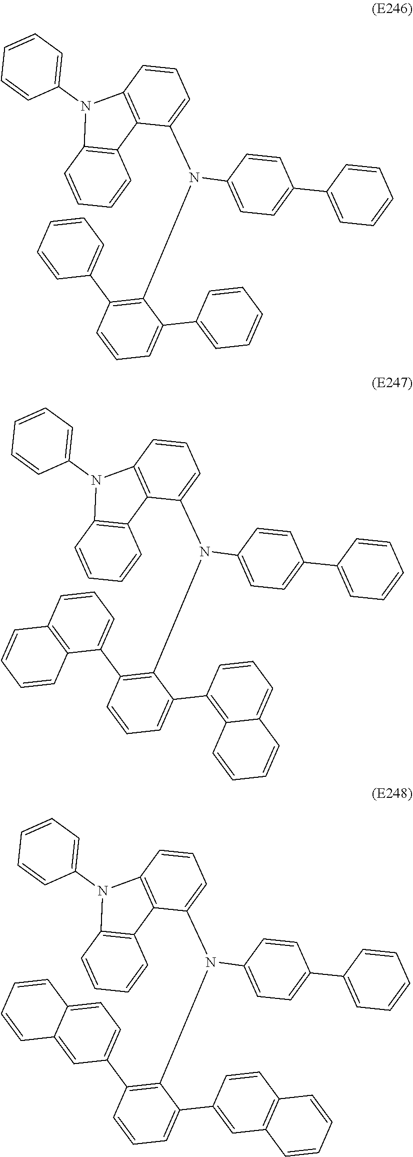 [SMILES: c1ccc(-c2ccc(N(c3c(-c4ccccc4)cccc3-c3ccccc3)c3cccc4c3c3ccccc3n4-c3ccccc3)cc2)cc1 ; c1ccc(-c2ccc(N(c3c(-c4cccc5ccccc45)cccc3-c3cccc4ccccc34)c3cccc4c3c3ccccc3n4-c3ccccc3)cc2)cc1 ; c1ccc(-c2ccc(N(c3c(-c4ccc5ccccc5c4)cccc3-c3ccc4ccccc4c3)c3cccc4c3c3ccccc3n4-c3ccccc3)cc2)cc1]

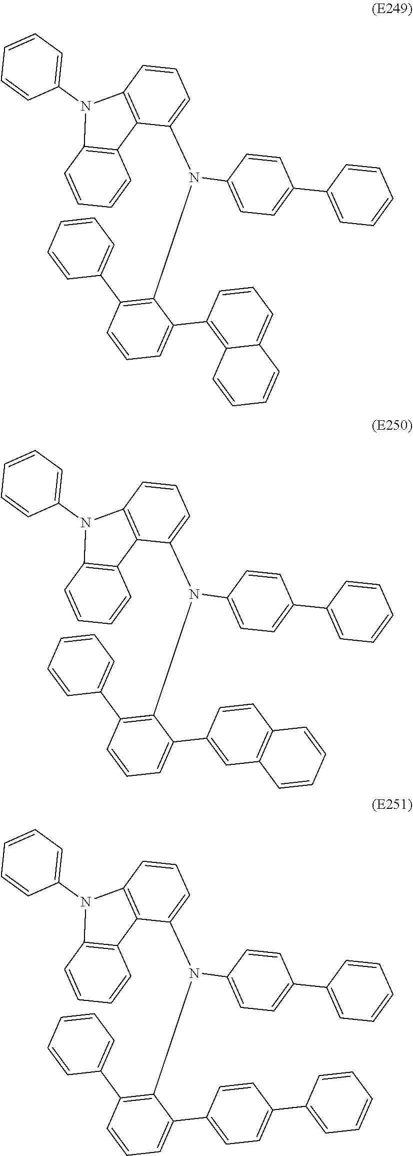 [SMILES: c1ccc(-c2ccc(N(c3c(-c4ccccc4)cccc3-c3cccc4ccccc34)c3cccc4c3c3ccccc3n4-c3ccccc3)cc2)cc1 ; c1ccc(-c2ccc(N(c3c(-c4ccccc4)cccc3-c3ccc4ccccc4c3)c3cccc4c3c3ccccc3n4-c3ccccc3)cc2)cc1 ; c1ccc(-c2ccc(-c3cccc(-c4ccccc4)c3N(c3ccc(-c4ccccc4)cc3)c3cccc4c3c3ccccc3n4-c3ccccc3)cc2)cc1]

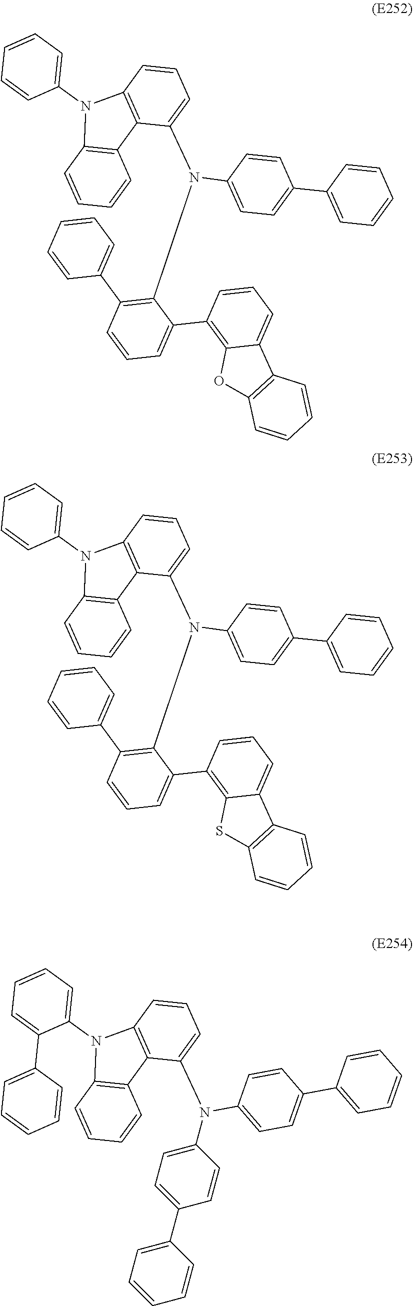 [SMILES: c1ccc(-c2ccc(N(c3c(-c4ccccc4)cccc3-c3cccc4c3oc3ccccc34)c3cccc4c3c3ccccc3n4-c3ccccc3)cc2)cc1 ; c1ccc(-c2ccc(N(c3c(-c4ccccc4)cccc3-c3cccc4c3sc3ccccc34)c3cccc4c3c3ccccc3n4-c3ccccc3)cc2)cc1 ; c1ccc(-c2ccc(N(c3ccc(-c4ccccc4)cc3)c3cccc4c3c3ccccc3n4-c3ccccc3-c3ccccc3)cc2)cc1]

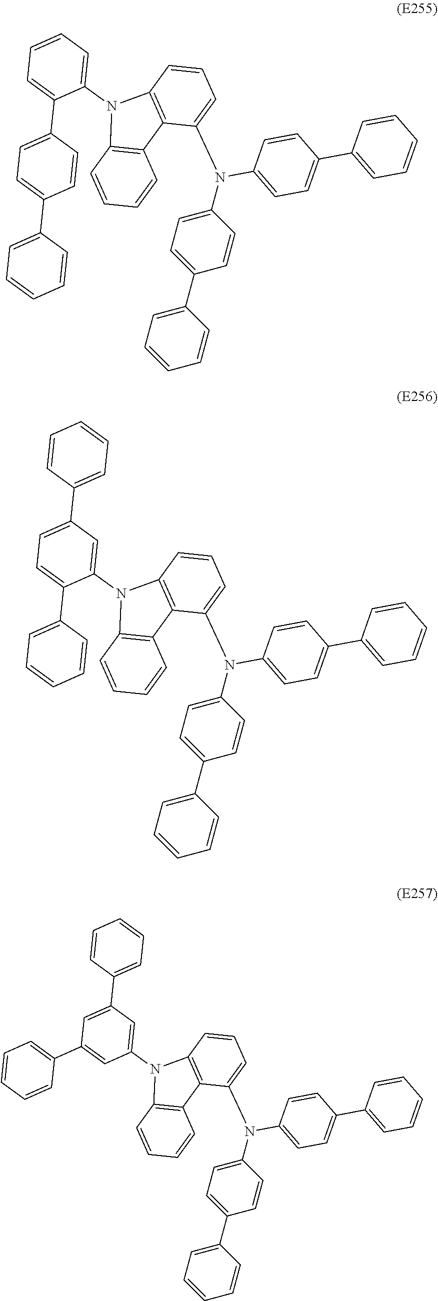 [SMILES: c1ccc(-c2ccc(-c3ccccc3-n3c4ccccc4c4c(N(c5ccc(-c6ccccc6)cc5)c5ccc(-c6ccccc6)cc5)cccc43)cc2)cc1 ; c1ccc(-c2ccc(N(c3ccc(-c4ccccc4)cc3)c3cccc4c3c3ccccc3n4-c3cc(-c4ccccc4)ccc3-c3ccccc3)cc2)cc1 ; c1ccc(-c2ccc(N(c3ccc(-c4ccccc4)cc3)c3cccc4c3c3ccccc3n4-c3cc(-c4ccccc4)cc(-c4ccccc4)c3)cc2)cc1]

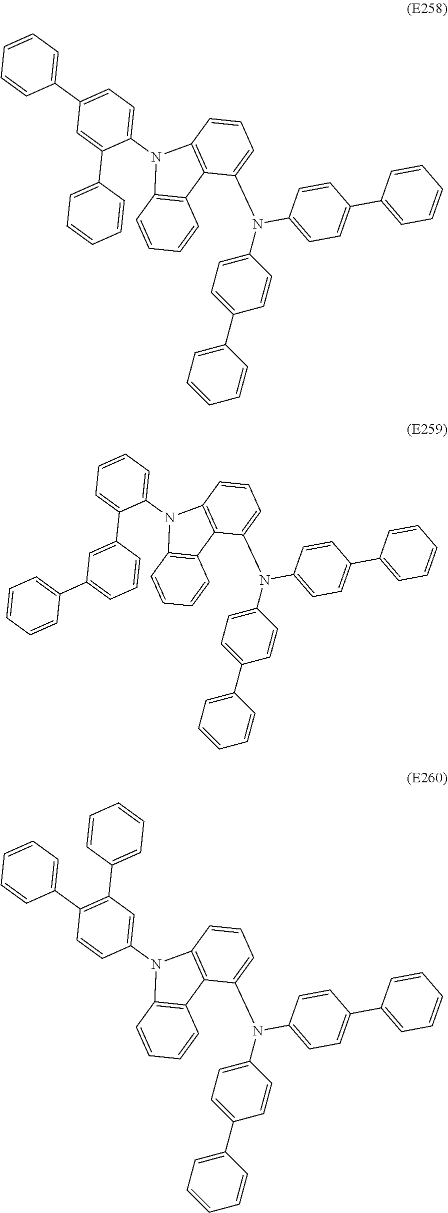 [SMILES: c1ccc(-c2ccc(N(c3ccc(-c4ccccc4)cc3)c3cccc4c3c3ccccc3n4-c3ccc(-c4ccccc4)cc3-c3ccccc3)cc2)cc1 ; c1ccc(-c2ccc(N(c3ccc(-c4ccccc4)cc3)c3cccc4c3c3ccccc3n4-c3ccccc3-c3cccc(-c4ccccc4)c3)cc2)cc1 ; c1ccc(-c2ccc(N(c3ccc(-c4ccccc4)cc3)c3cccc4c3c3ccccc3n4-c3ccc(-c4ccccc4)c(-c4ccccc4)c3)cc2)cc1]

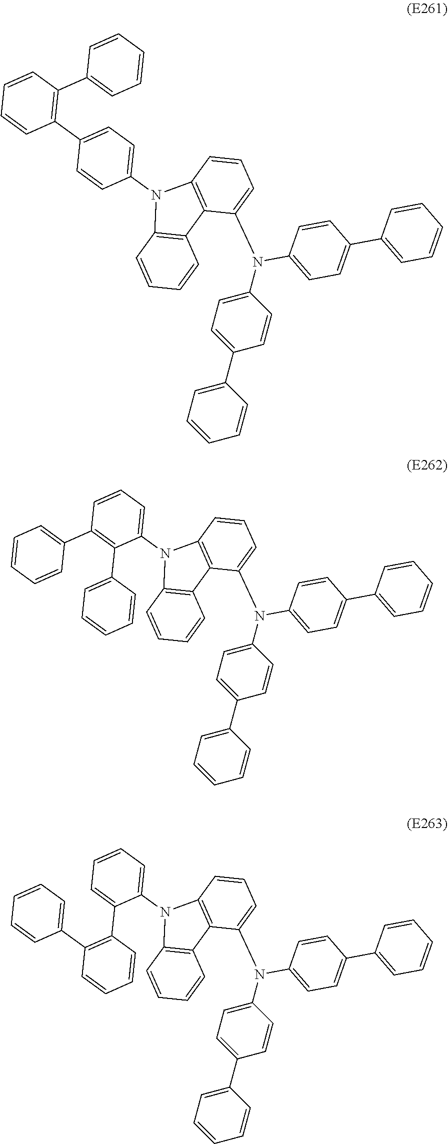 [SMILES: c1ccc(-c2ccc(N(c3ccc(-c4ccccc4)cc3)c3cccc4c3c3ccccc3n4-c3ccc(-c4ccccc4-c4ccccc4)cc3)cc2)cc1 ; c1ccc(-c2ccc(N(c3ccc(-c4ccccc4)cc3)c3cccc4c3c3ccccc3n4-c3cccc(-c4ccccc4)c3-c3ccccc3)cc2)cc1 ; c1ccc(-c2ccc(N(c3ccc(-c4ccccc4)cc3)c3cccc4c3c3ccccc3n4-c3ccccc3-c3ccccc3-c3ccccc3)cc2)cc1]

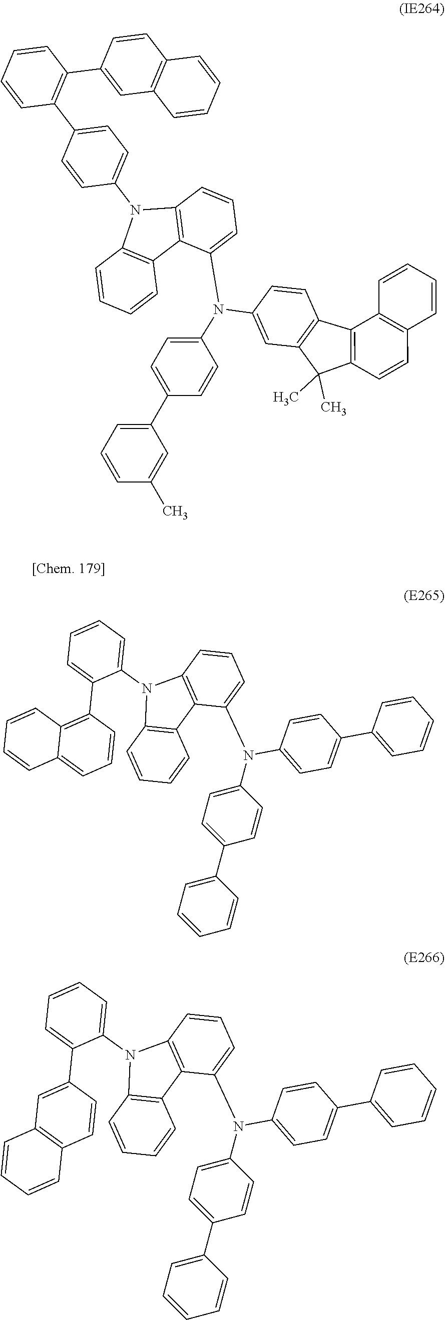 [SMILES: Cc1cccc(-c2ccc(N(c3ccc4c(c3)C(C)(C)c3ccc5ccccc5c3-4)c3cccc4c3c3ccccc3n4-c3ccc(-c4ccccc4-c4ccc5ccccc5c4)cc3)cc2)c1 ; c1ccc(-c2ccc(N(c3ccc(-c4ccccc4)cc3)c3cccc4c3c3ccccc3n4-c3ccccc3-c3cccc4ccccc34)cc2)cc1 ; c1ccc(-c2ccc(N(c3ccc(-c4ccccc4)cc3)c3cccc4c3c3ccccc3n4-c3ccccc3-c3ccc4ccccc4c3)cc2)cc1]

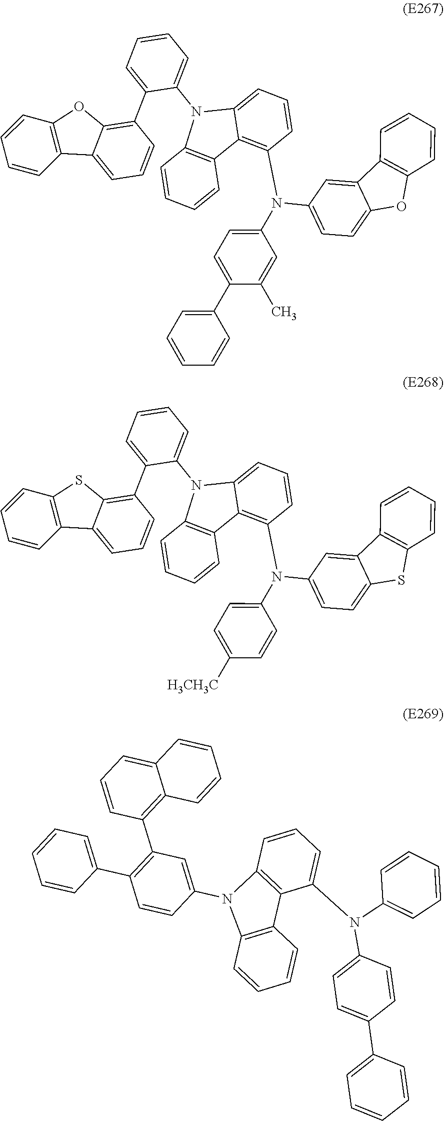

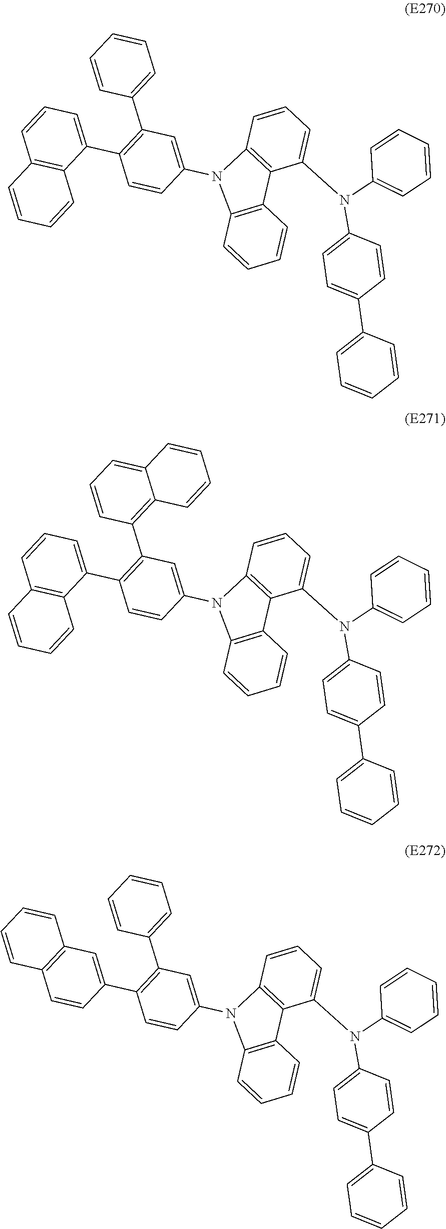 [SMILES: c1ccc(-c2ccc(N(c3ccccc3)c3cccc4c3c3ccccc3n4-c3ccc(-c4cccc5ccccc45)c(-c4ccccc4)c3)cc2)cc1 ; c1ccc(-c2ccc(N(c3ccccc3)c3cccc4c3c3ccccc3n4-c3ccc(-c4cccc5ccccc45)c(-c4cccc5ccccc45)c3)cc2)cc1 ; c1ccc(-c2ccc(N(c3ccccc3)c3cccc4c3c3ccccc3n4-c3ccc(-c4ccc5ccccc5c4)c(-c4ccccc4)c3)cc2)cc1]

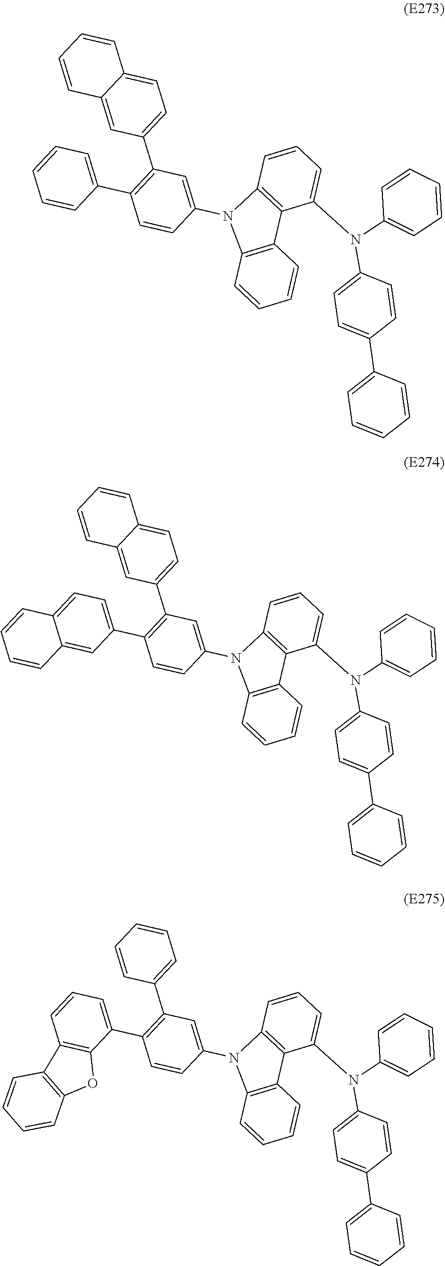 [SMILES: c1ccc(-c2ccc(N(c3ccccc3)c3cccc4c3c3ccccc3n4-c3ccc(-c4ccccc4)c(-c4ccc5ccccc5c4)c3)cc2)cc1 ; c1ccc(-c2ccc(N(c3ccccc3)c3cccc4c3c3ccccc3n4-c3ccc(-c4ccc5ccccc5c4)c(-c4ccc5ccccc5c4)c3)cc2)cc1 ; c1ccc(-c2ccc(N(c3ccccc3)c3cccc4c3c3ccccc3n4-c3ccc(-c4cccc5c4oc4ccccc45)c(-c4ccccc4)c3)cc2)cc1]

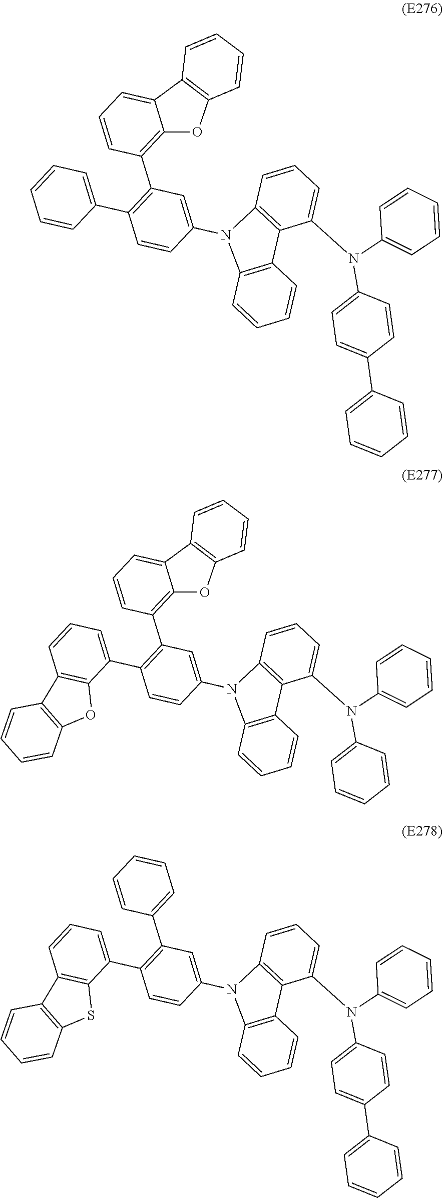 [SMILES: c1ccc(-c2ccc(N(c3ccccc3)c3cccc4c3c3ccccc3n4-c3ccc(-c4ccccc4)c(-c4cccc5c4oc4ccccc45)c3)cc2)cc1 ; c1ccc(N(c2ccccc2)c2cccc3c2c2ccccc2n3-c2ccc(-c3cccc4c3oc3ccccc34)c(-c3cccc4c3oc3ccccc34)c2)cc1 ; c1ccc(-c2ccc(N(c3ccccc3)c3cccc4c3c3ccccc3n4-c3ccc(-c4cccc5c4sc4ccccc45)c(-c4ccccc4)c3)cc2)cc1]

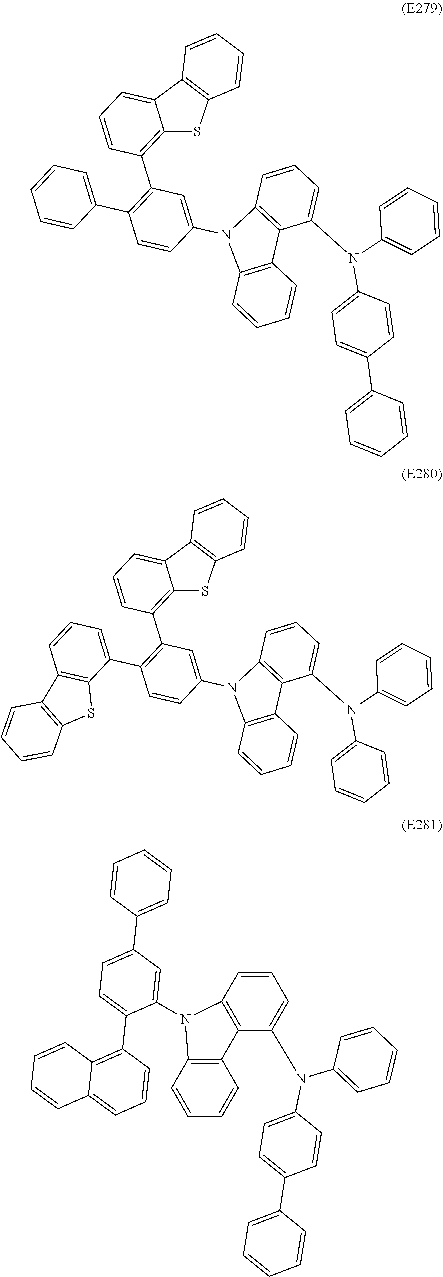 [SMILES: c1ccc(-c2ccc(N(c3ccccc3)c3cccc4c3c3ccccc3n4-c3ccc(-c4ccccc4)c(-c4cccc5c4sc4ccccc45)c3)cc2)cc1 ; c1ccc(N(c2ccccc2)c2cccc3c2c2ccccc2n3-c2ccc(-c3cccc4c3sc3ccccc34)c(-c3cccc4c3sc3ccccc34)c2)cc1 ; c1ccc(-c2ccc(N(c3ccccc3)c3cccc4c3c3ccccc3n4-c3cc(-c4ccccc4)ccc3-c3cccc4ccccc34)cc2)cc1]

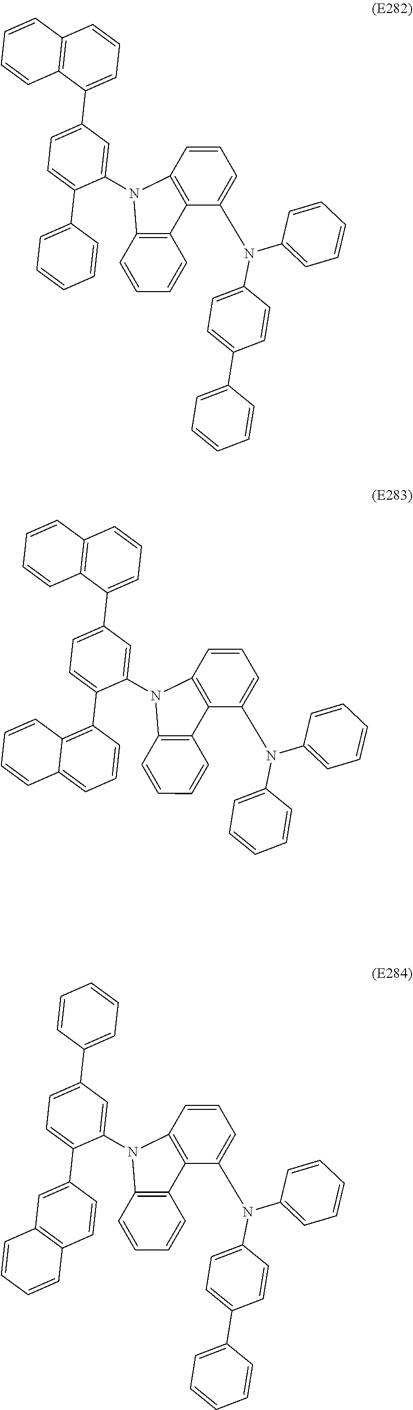 [SMILES: c1ccc(-c2ccc(N(c3ccccc3)c3cccc4c3c3ccccc3n4-c3cc(-c4cccc5ccccc45)ccc3-c3ccccc3)cc2)cc1 ; c1ccc(N(c2ccccc2)c2cccc3c2c2ccccc2n3-c2cc(-c3cccc4ccccc34)ccc2-c2cccc3ccccc23)cc1 ; c1ccc(-c2ccc(N(c3ccccc3)c3cccc4c3c3ccccc3n4-c3cc(-c4ccccc4)ccc3-c3ccc4ccccc4c3)cc2)cc1]

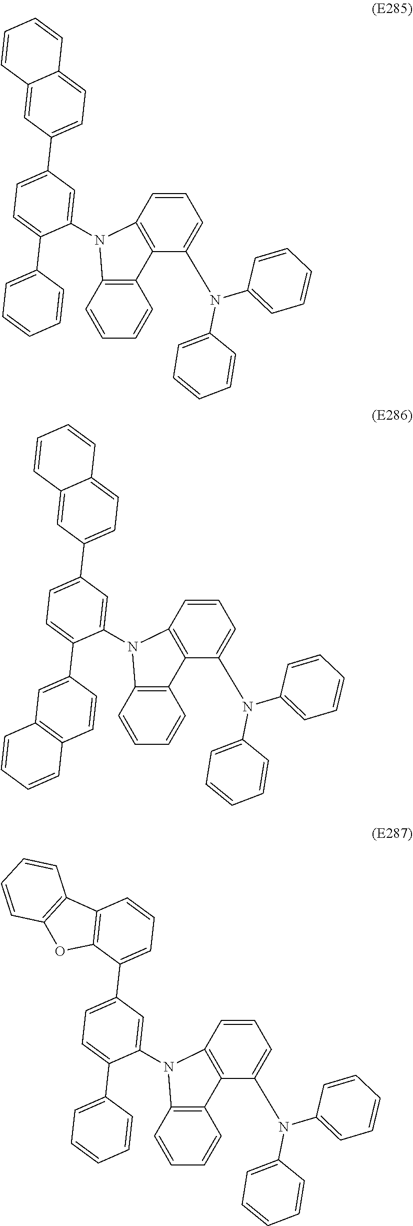 [SMILES: c1ccc(-c2ccc(-c3ccc4ccccc4c3)cc2-n2c3ccccc3c3c(N(c4ccccc4)c4ccccc4)cccc32)cc1 ; c1ccc(N(c2ccccc2)c2cccc3c2c2ccccc2n3-c2cc(-c3ccc4ccccc4c3)ccc2-c2ccc3ccccc3c2)cc1 ; c1ccc(-c2ccc(-c3cccc4c3oc3ccccc34)cc2-n2c3ccccc3c3c(N(c4ccccc4)c4ccccc4)cccc32)cc1]

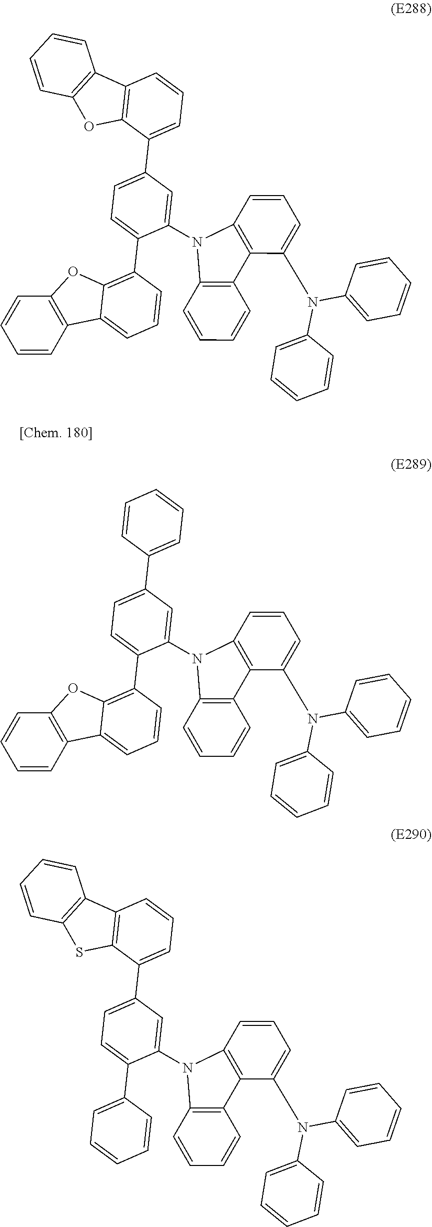 [SMILES: c1ccc(N(c2ccccc2)c2cccc3c2c2ccccc2n3-c2cc(-c3cccc4c3oc3ccccc34)ccc2-c2cccc3c2oc2ccccc23)cc1 ; c1ccc(-c2ccc(-c3cccc4c3oc3ccccc34)c(-n3c4ccccc4c4c(N(c5ccccc5)c5ccccc5)cccc43)c2)cc1 ; c1ccc(-c2ccc(-c3cccc4c3sc3ccccc34)cc2-n2c3ccccc3c3c(N(c4ccccc4)c4ccccc4)cccc32)cc1]

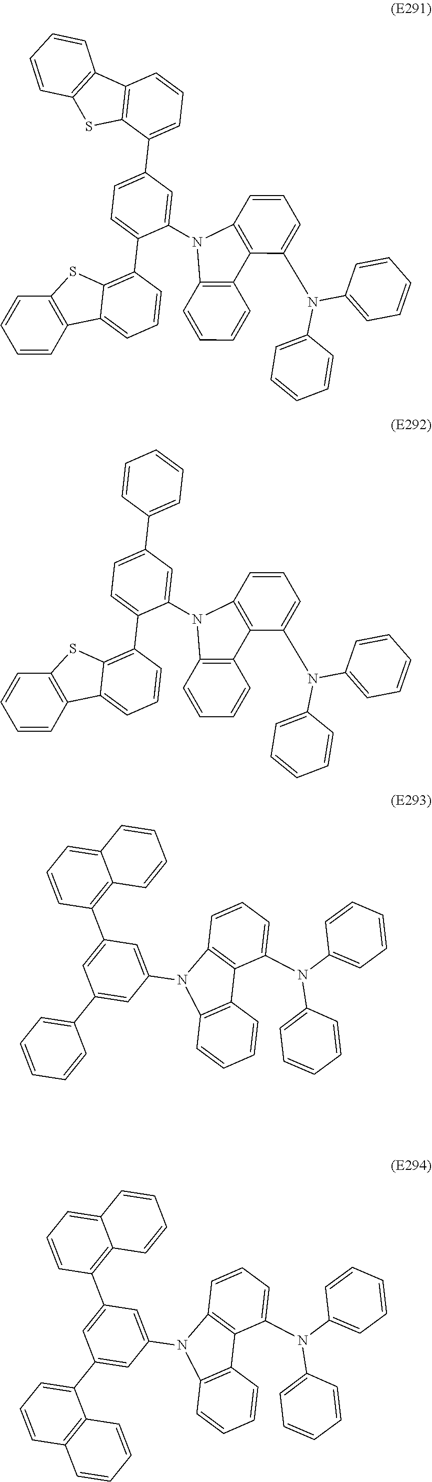 [SMILES: c1ccc(N(c2ccccc2)c2cccc3c2c2ccccc2n3-c2cc(-c3cccc4c3sc3ccccc34)ccc2-c2cccc3c2sc2ccccc23)cc1 ; c1ccc(-c2ccc(-c3cccc4c3sc3ccccc34)c(-n3c4ccccc4c4c(N(c5ccccc5)c5ccccc5)cccc43)c2)cc1 ; c1ccc(-c2cc(-c3cccc4ccccc34)cc(-n3c4ccccc4c4c(N(c5ccccc5)c5ccccc5)cccc43)c2)cc1 ; c1ccc(N(c2ccccc2)c2cccc3c2c2ccccc2n3-c2cc(-c3cccc4ccccc34)cc(-c3cccc4ccccc34)c2)cc1]

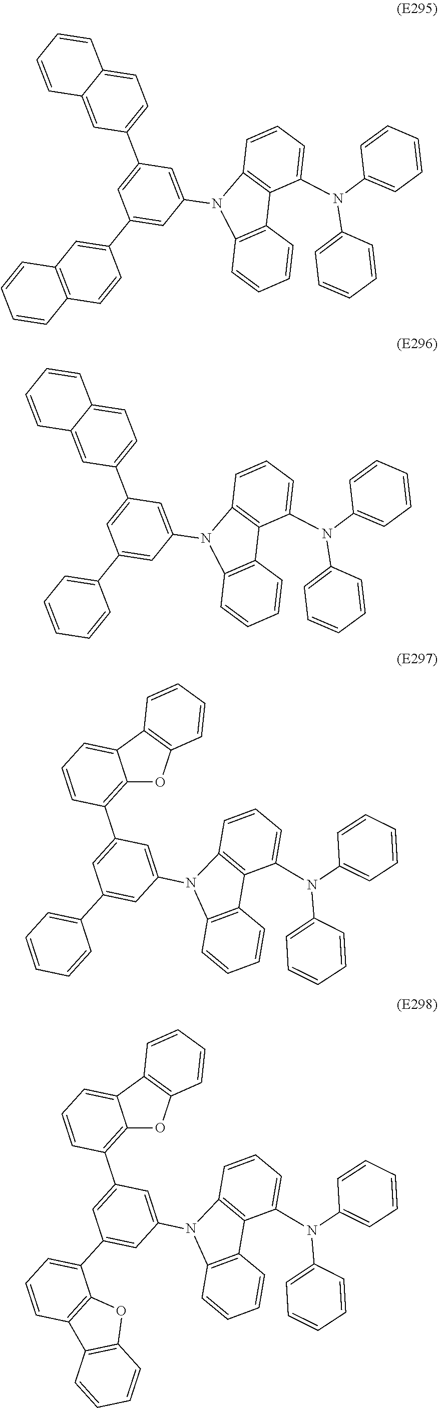

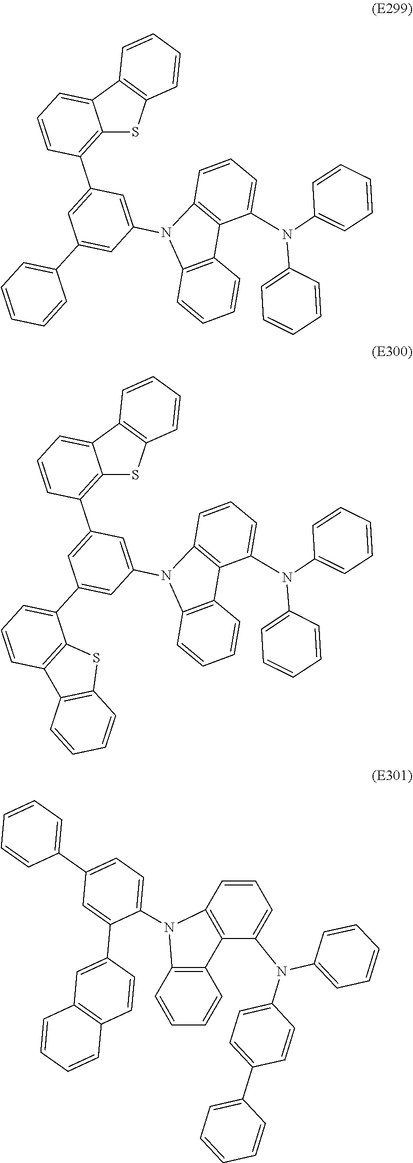 [SMILES: c1ccc(-c2cc(-c3cccc4c3sc3ccccc34)cc(-n3c4ccccc4c4c(N(c5ccccc5)c5ccccc5)cccc43)c2)cc1 ; c1ccc(N(c2ccccc2)c2cccc3c2c2ccccc2n3-c2cc(-c3cccc4c3sc3ccccc34)cc(-c3cccc4c3sc3ccccc34)c2)cc1 ; c1ccc(-c2ccc(N(c3ccccc3)c3cccc4c3c3ccccc3n4-c3ccc(-c4ccccc4)cc3-c3ccc4ccccc4c3)cc2)cc1]

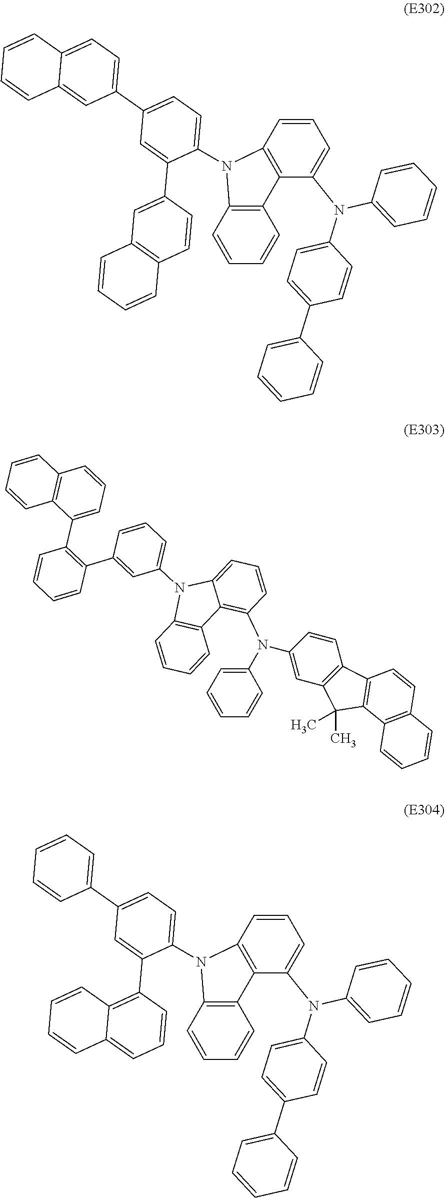 [SMILES: c1ccc(-c2ccc(N(c3ccccc3)c3cccc4c3c3ccccc3n4-c3ccc(-c4ccc5ccccc5c4)cc3-c3ccc4ccccc4c3)cc2)cc1 ; CC1(C)c2cc(N(c3ccccc3)c3cccc4c3c3ccccc3n4-c3cccc(-c4ccccc4-c4cccc5ccccc45)c3)ccc2-c2ccc3ccccc3c21 ; c1ccc(-c2ccc(N(c3ccccc3)c3cccc4c3c3ccccc3n4-c3ccc(-c4ccccc4)cc3-c3cccc4ccccc34)cc2)cc1]

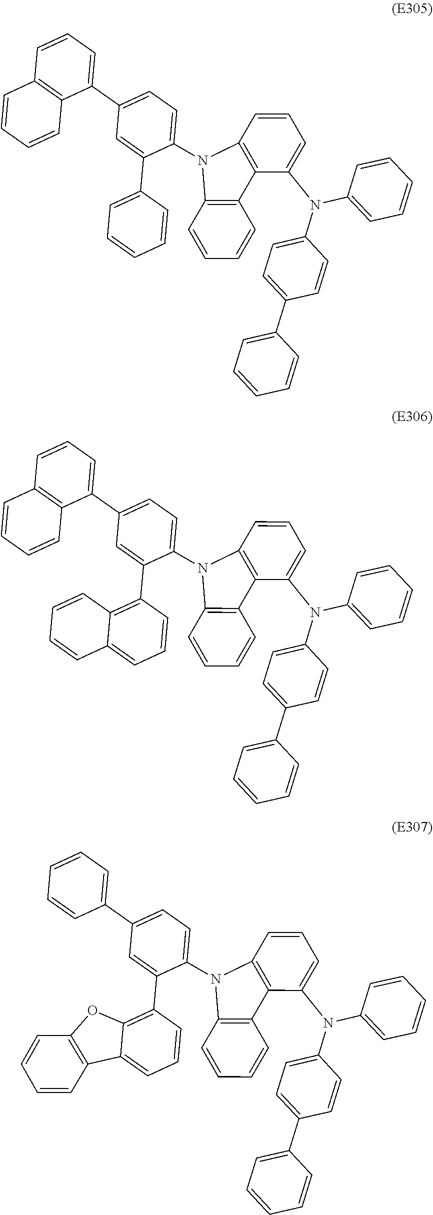 [SMILES: c1ccc(-c2ccc(N(c3ccccc3)c3cccc4c3c3ccccc3n4-c3ccc(-c4cccc5ccccc45)cc3-c3ccccc3)cc2)cc1 ; c1ccc(-c2ccc(N(c3ccccc3)c3cccc4c3c3ccccc3n4-c3ccc(-c4cccc5ccccc45)cc3-c3cccc4ccccc34)cc2)cc1 ; c1ccc(-c2ccc(N(c3ccccc3)c3cccc4c3c3ccccc3n4-c3ccc(-c4ccccc4)cc3-c3cccc4c3oc3ccccc34)cc2)cc1]

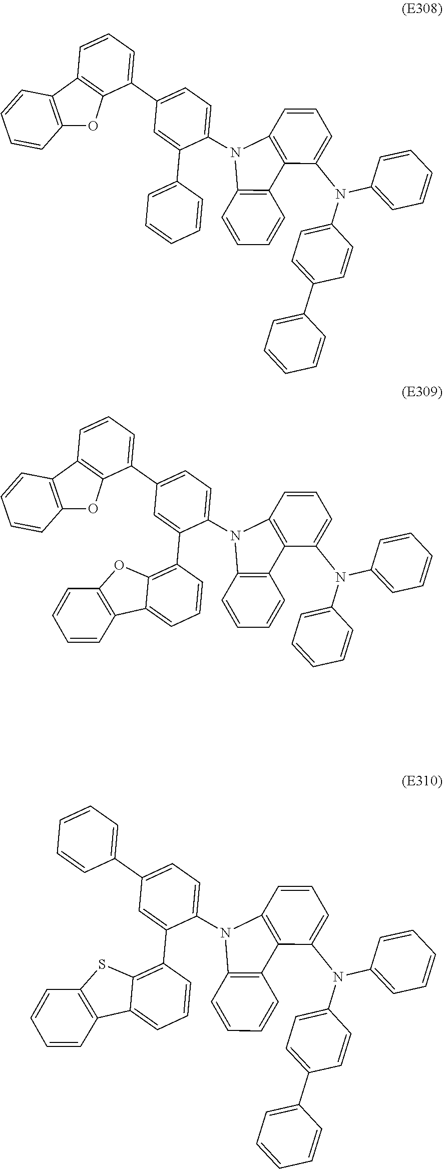 [SMILES: c1ccc(-c2ccc(N(c3ccccc3)c3cccc4c3c3ccccc3n4-c3ccc(-c4cccc5c4oc4ccccc45)cc3-c3ccccc3)cc2)cc1 ; c1ccc(N(c2ccccc2)c2cccc3c2c2ccccc2n3-c2ccc(-c3cccc4c3oc3ccccc34)cc2-c2cccc3c2oc2ccccc23)cc1 ; c1ccc(-c2ccc(N(c3ccccc3)c3cccc4c3c3ccccc3n4-c3ccc(-c4ccccc4)cc3-c3cccc4c3sc3ccccc34)cc2)cc1]

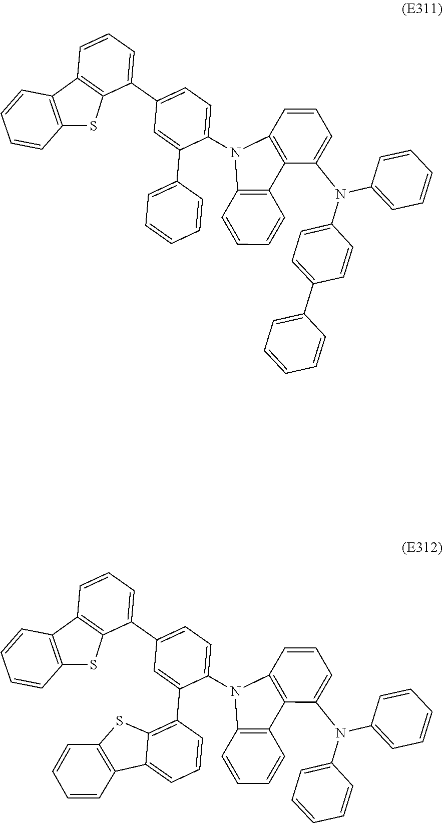 [SMILES: c1ccc(-c2ccc(N(c3ccccc3)c3cccc4c3c3ccccc3n4-c3ccc(-c4cccc5c4sc4ccccc45)cc3-c3ccccc3)cc2)cc1 ; c1ccc(N(c2ccccc2)c2cccc3c2c2ccccc2n3-c2ccc(-c3cccc4c3sc3ccccc34)cc2-c2cccc3c2sc2ccccc23)cc1]

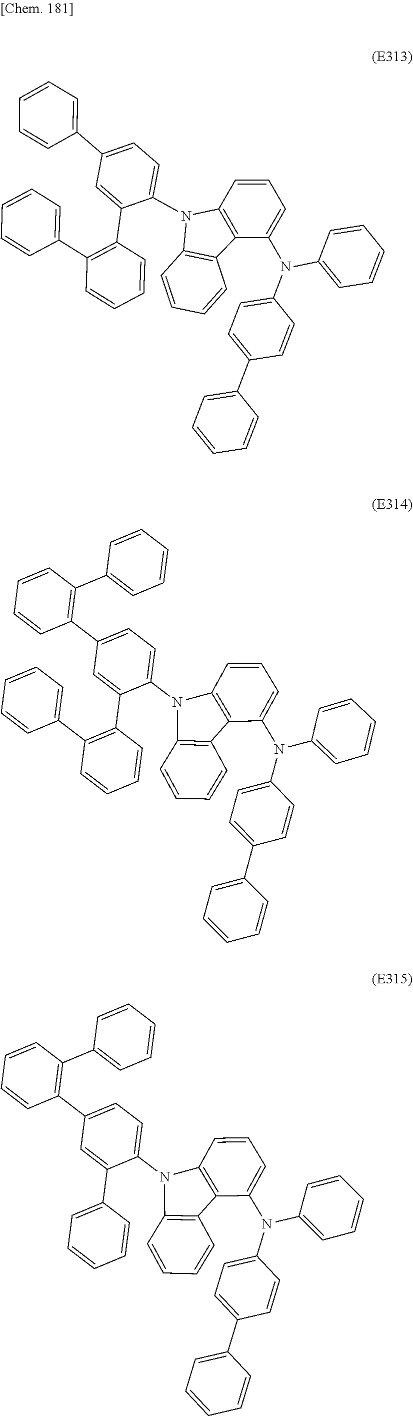 [SMILES: c1ccc(-c2ccc(N(c3ccccc3)c3cccc4c3c3ccccc3n4-c3ccc(-c4ccccc4)cc3-c3ccccc3-c3ccccc3)cc2)cc1 ; c1ccc(-c2ccc(N(c3ccccc3)c3cccc4c3c3ccccc3n4-c3ccc(-c4ccccc4-c4ccccc4)cc3-c3ccccc3-c3ccccc3)cc2)cc1 ; c1ccc(-c2ccc(N(c3ccccc3)c3cccc4c3c3ccccc3n4-c3ccc(-c4ccccc4-c4ccccc4)cc3-c3ccccc3)cc2)cc1]

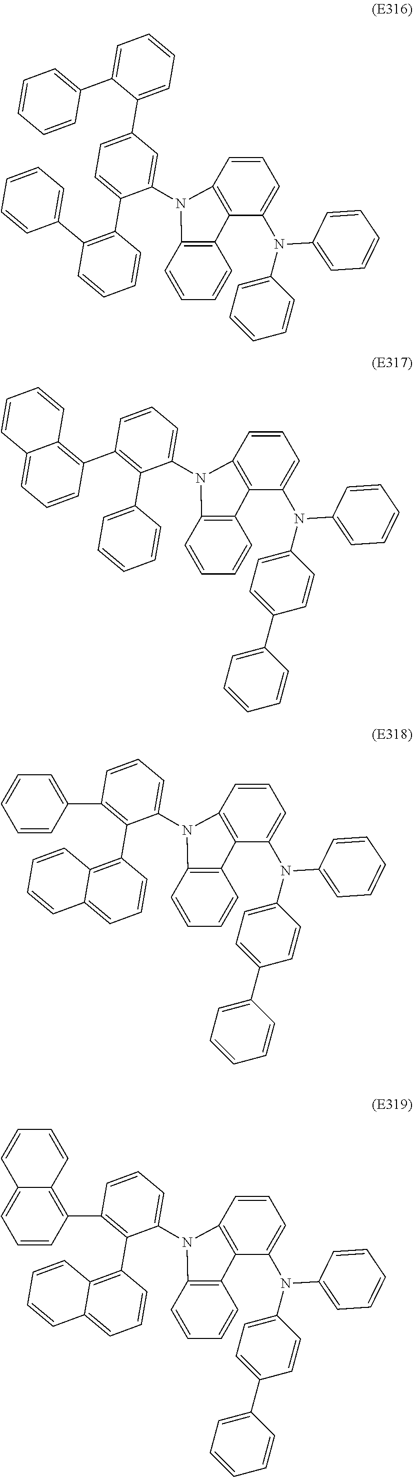 [SMILES: c1ccc(-c2ccccc2-c2ccc(-c3ccccc3-c3ccccc3)c(-n3c4ccccc4c4c(N(c5ccccc5)c5ccccc5)cccc43)c2)cc1 ; c1ccc(-c2ccc(N(c3ccccc3)c3cccc4c3c3ccccc3n4-c3cccc(-c4cccc5ccccc45)c3-c3ccccc3)cc2)cc1 ; c1ccc(-c2ccc(N(c3ccccc3)c3cccc4c3c3ccccc3n4-c3cccc(-c4ccccc4)c3-c3cccc4ccccc34)cc2)cc1 ; c1ccc(-c2ccc(N(c3ccccc3)c3cccc4c3c3ccccc3n4-c3cccc(-c4cccc5ccccc45)c3-c3cccc4ccccc34)cc2)cc1]

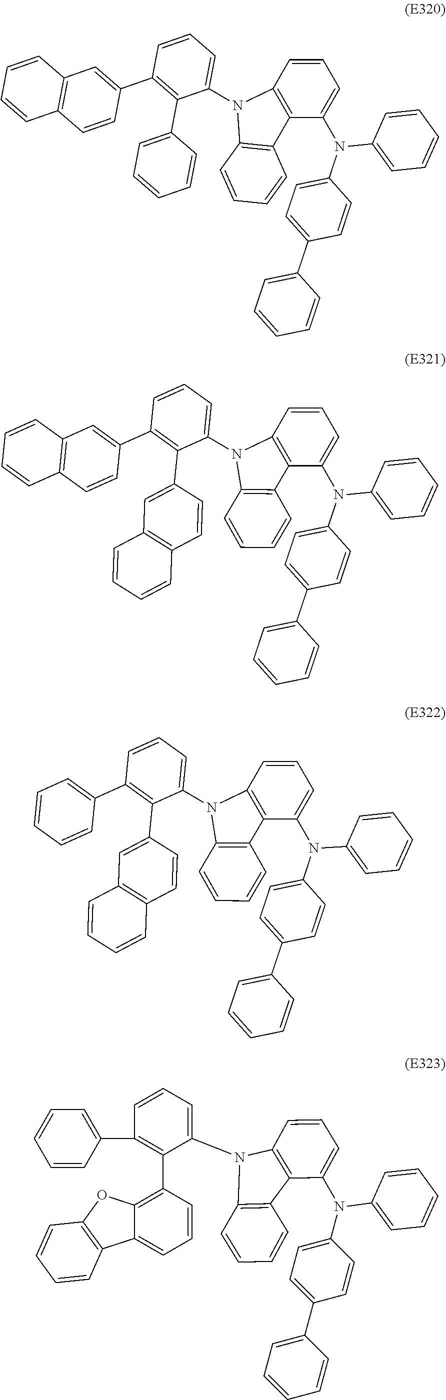 [SMILES: c1ccc(-c2ccc(N(c3ccccc3)c3cccc4c3c3ccccc3n4-c3cccc(-c4ccc5ccccc5c4)c3-c3ccccc3)cc2)cc1 ; c1ccc(-c2ccc(N(c3ccccc3)c3cccc4c3c3ccccc3n4-c3cccc(-c4ccc5ccccc5c4)c3-c3ccc4ccccc4c3)cc2)cc1 ; c1ccc(-c2ccc(N(c3ccccc3)c3cccc4c3c3ccccc3n4-c3cccc(-c4ccccc4)c3-c3ccc4ccccc4c3)cc2)cc1 ; c1ccc(-c2ccc(N(c3ccccc3)c3cccc4c3c3ccccc3n4-c3cccc(-c4ccccc4)c3-c3cccc4c3oc3ccccc34)cc2)cc1]

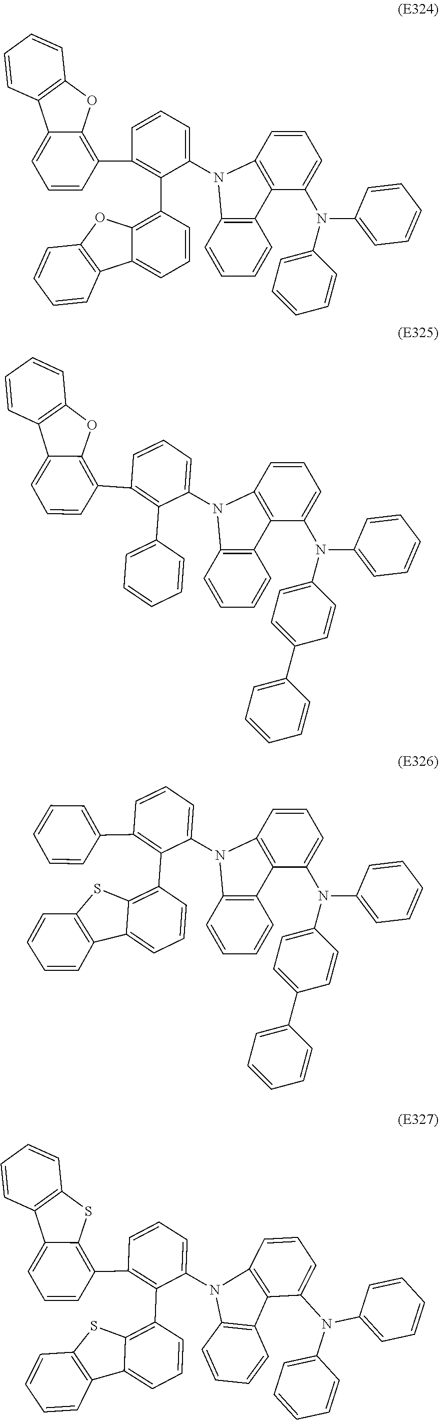 [SMILES: c1ccc(N(c2ccccc2)c2cccc3c2c2ccccc2n3-c2cccc(-c3cccc4c3oc3ccccc34)c2-c2cccc3c2oc2ccccc23)cc1 ; c1ccc(-c2ccc(N(c3ccccc3)c3cccc4c3c3ccccc3n4-c3cccc(-c4cccc5c4oc4ccccc45)c3-c3ccccc3)cc2)cc1 ; c1ccc(-c2ccc(N(c3ccccc3)c3cccc4c3c3ccccc3n4-c3cccc(-c4ccccc4)c3-c3cccc4c3sc3ccccc34)cc2)cc1 ; c1ccc(N(c2ccccc2)c2cccc3c2c2ccccc2n3-c2cccc(-c3cccc4c3sc3ccccc34)c2-c2cccc3c2sc2ccccc23)cc1]

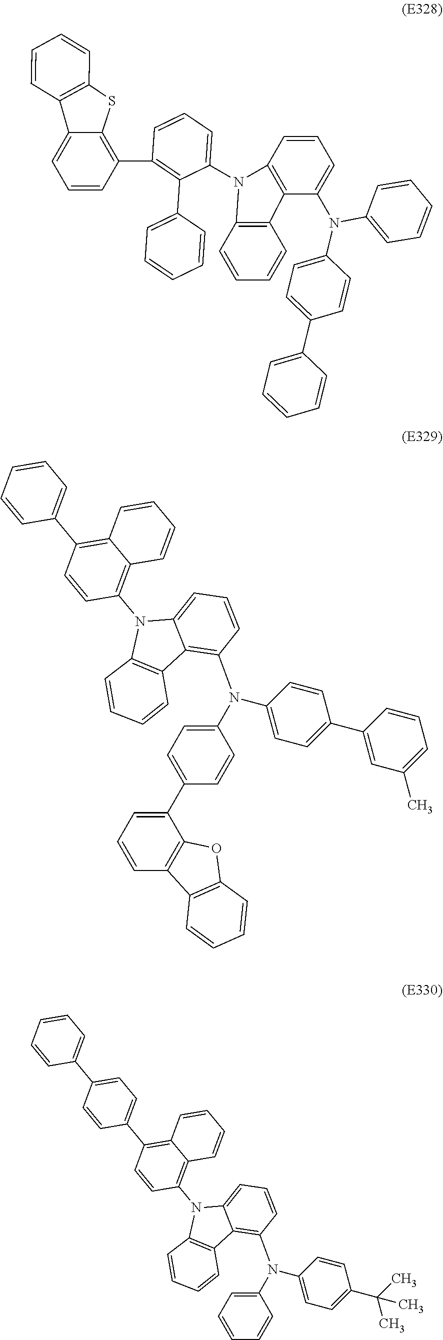 [SMILES: c1ccc(-c2ccc(N(c3ccccc3)c3cccc4c3c3ccccc3n4-c3cccc(-c4cccc5c4sc4ccccc45)c3-c3ccccc3)cc2)cc1 ; Cc1cccc(-c2ccc(N(c3ccc(-c4cccc5c4oc4ccccc45)cc3)c3cccc4c3c3ccccc3n4-c3ccc(-c4ccccc4)c4ccccc34)cc2)c1 ; CC(C)(C)c1ccc(N(c2ccccc2)c2cccc3c2c2ccccc2n3-c2ccc(-c3ccc(-c4ccccc4)cc3)c3ccccc23)cc1]

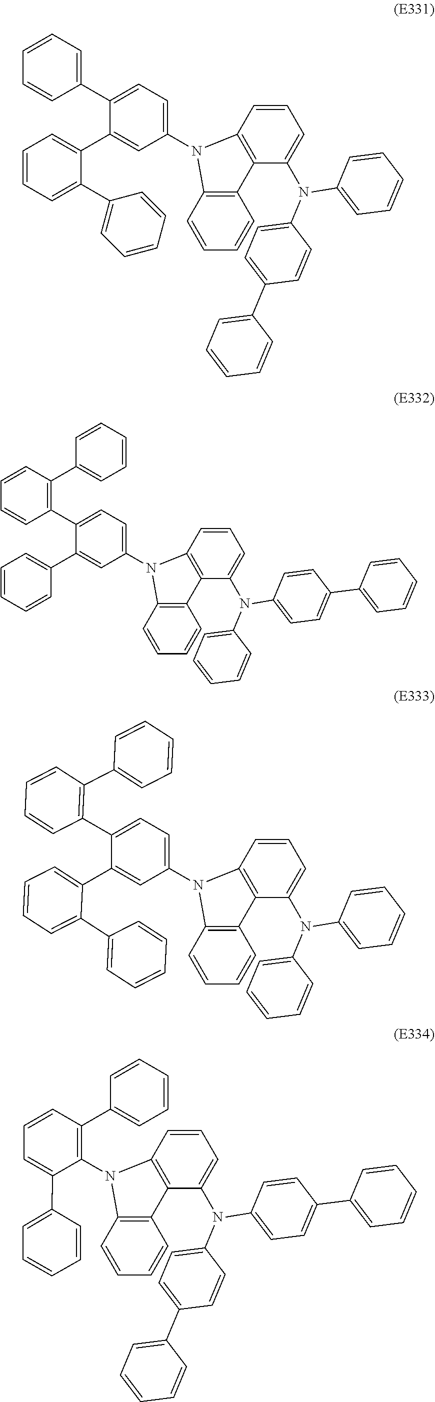 [SMILES: c1ccc(-c2ccc(N(c3ccccc3)c3cccc4c3c3ccccc3n4-c3ccc(-c4ccccc4)c(-c4ccccc4-c4ccccc4)c3)cc2)cc1 ; c1ccc(-c2ccc(N(c3ccccc3)c3cccc4c3c3ccccc3n4-c3ccc(-c4ccccc4-c4ccccc4)c(-c4ccccc4)c3)cc2)cc1 ; c1ccc(-c2ccccc2-c2ccc(-n3c4ccccc4c4c(N(c5ccccc5)c5ccccc5)cccc43)cc2-c2ccccc2-c2ccccc2)cc1 ; c1ccc(-c2ccc(N(c3ccc(-c4ccccc4)cc3)c3cccc4c3c3ccccc3n4-c3c(-c4ccccc4)cccc3-c3ccccc3)cc2)cc1]

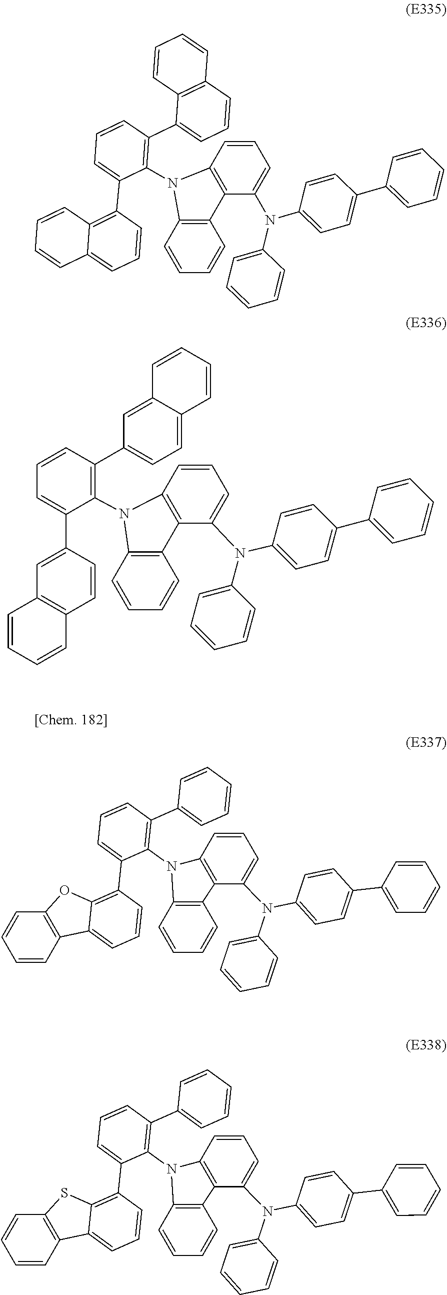 [SMILES: c1ccc(-c2ccc(N(c3ccccc3)c3cccc4c3c3ccccc3n4-c3c(-c4cccc5ccccc45)cccc3-c3cccc4ccccc34)cc2)cc1 ; c1ccc(-c2ccc(N(c3ccccc3)c3cccc4c3c3ccccc3n4-c3c(-c4ccc5ccccc5c4)cccc3-c3ccc4ccccc4c3)cc2)cc1 ; c1ccc(-c2ccc(N(c3ccccc3)c3cccc4c3c3ccccc3n4-c3c(-c4ccccc4)cccc3-c3cccc4c3oc3ccccc34)cc2)cc1 ; c1ccc(-c2ccc(N(c3ccccc3)c3cccc4c3c3ccccc3n4-c3c(-c4ccccc4)cccc3-c3cccc4c3sc3ccccc34)cc2)cc1]

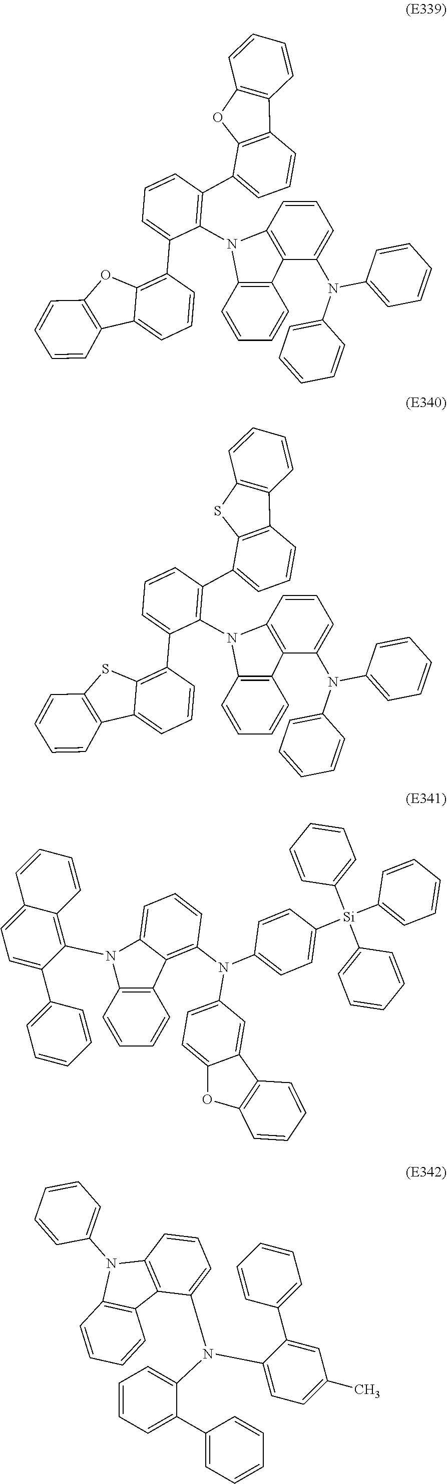 [SMILES: c1ccc(N(c2ccccc2)c2cccc3c2c2ccccc2n3-c2c(-c3cccc4c3oc3ccccc34)cccc2-c2cccc3c2oc2ccccc23)cc1 ; c1ccc(N(c2ccccc2)c2cccc3c2c2ccccc2n3-c2c(-c3cccc4c3sc3ccccc34)cccc2-c2cccc3c2sc2ccccc23)cc1 ; c1ccc(-c2ccc3ccccc3c2-n2c3ccccc3c3c(N(c4ccc([Si](c5ccccc5)(c5ccccc5)c5ccccc5)cc4)c4ccc5oc6ccccc6c5c4)cccc32)cc1 ; Cc1ccc(N(c2ccccc2-c2ccccc2)c2cccc3c2c2ccccc2n3-c2ccccc2)c(-c2ccccc2)c1]

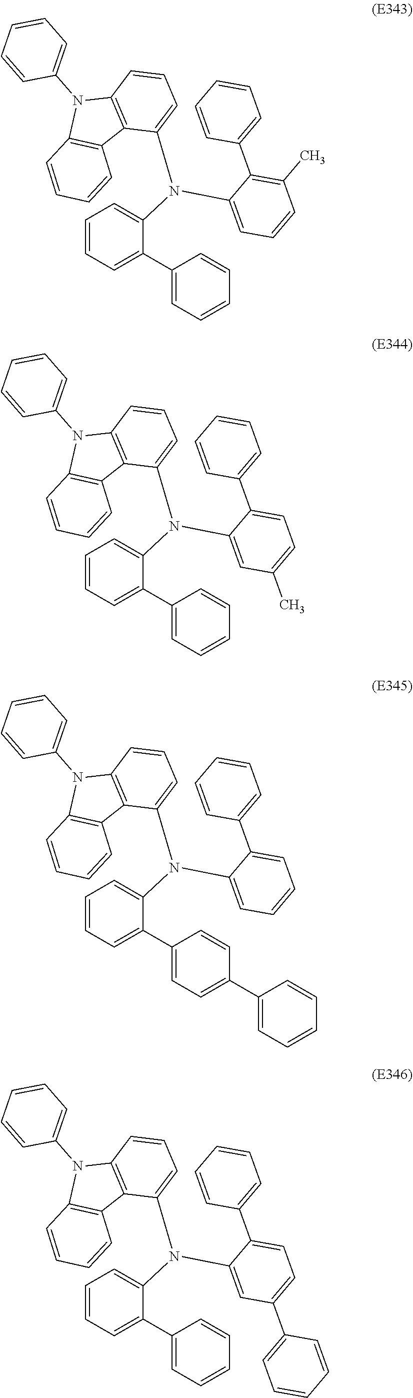 [SMILES: Cc1cccc(N(c2ccccc2-c2ccccc2)c2cccc3c2c2ccccc2n3-c2ccccc2)c1-c1ccccc1 ; Cc1ccc(-c2ccccc2)c(N(c2ccccc2-c2ccccc2)c2cccc3c2c2ccccc2n3-c2ccccc2)c1 ; c1ccc(-c2ccc(-c3ccccc3N(c3ccccc3-c3ccccc3)c3cccc4c3c3ccccc3n4-c3ccccc3)cc2)cc1 ; c1ccc(-c2ccc(-c3ccccc3)c(N(c3ccccc3-c3ccccc3)c3cccc4c3c3ccccc3n4-c3ccccc3)c2)cc1]

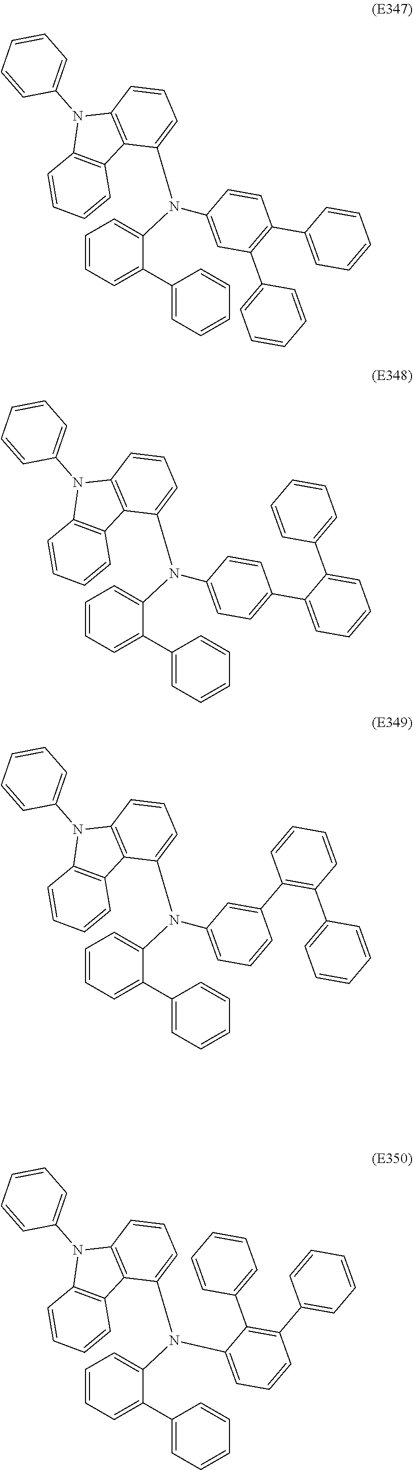 [SMILES: c1ccc(-c2ccc(N(c3ccccc3-c3ccccc3)c3cccc4c3c3ccccc3n4-c3ccccc3)cc2-c2ccccc2)cc1 ; c1ccc(-c2ccccc2-c2ccc(N(c3ccccc3-c3ccccc3)c3cccc4c3c3ccccc3n4-c3ccccc3)cc2)cc1 ; c1ccc(-c2ccccc2-c2cccc(N(c3ccccc3-c3ccccc3)c3cccc4c3c3ccccc3n4-c3ccccc3)c2)cc1 ; c1ccc(-c2ccccc2N(c2cccc(-c3ccccc3)c2-c2ccccc2)c2cccc3c2c2ccccc2n3-c2ccccc2)cc1]

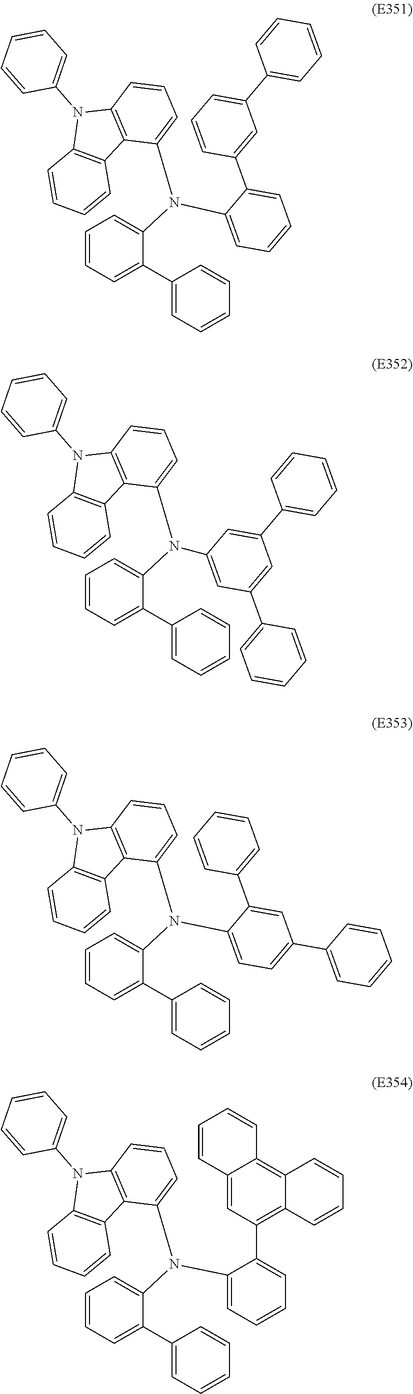 [SMILES: c1ccc(-c2cccc(-c3ccccc3N(c3ccccc3-c3ccccc3)c3cccc4c3c3ccccc3n4-c3ccccc3)c2)cc1 ; c1ccc(-c2cc(-c3ccccc3)cc(N(c3ccccc3-c3ccccc3)c3cccc4c3c3ccccc3n4-c3ccccc3)c2)cc1 ; c1ccc(-c2ccc(N(c3ccccc3-c3ccccc3)c3cccc4c3c3ccccc3n4-c3ccccc3)c(-c3ccccc3)c2)cc1 ; c1ccc(-c2ccccc2N(c2ccccc2-c2cc3ccccc3c3ccccc23)c2cccc3c2c2ccccc2n3-c2ccccc2)cc1]

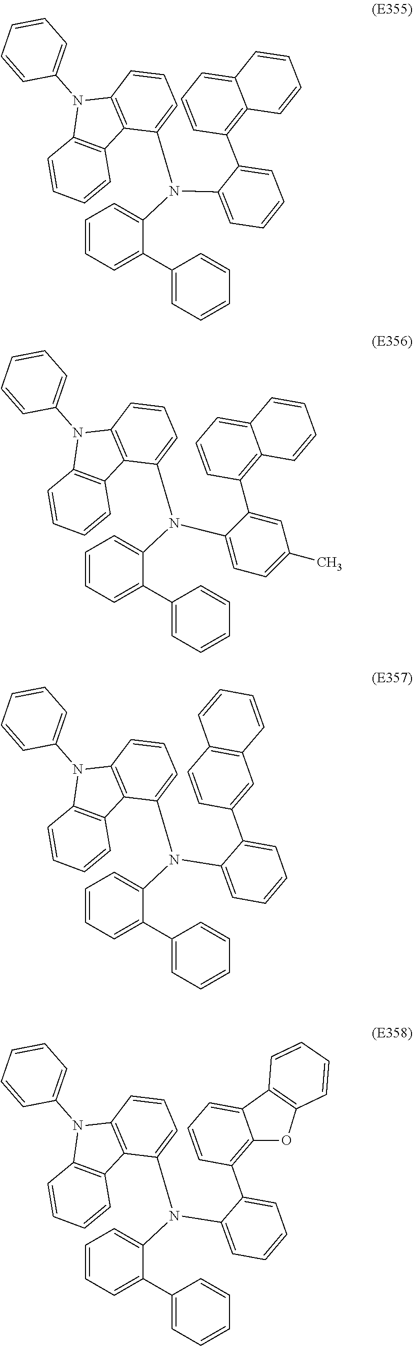

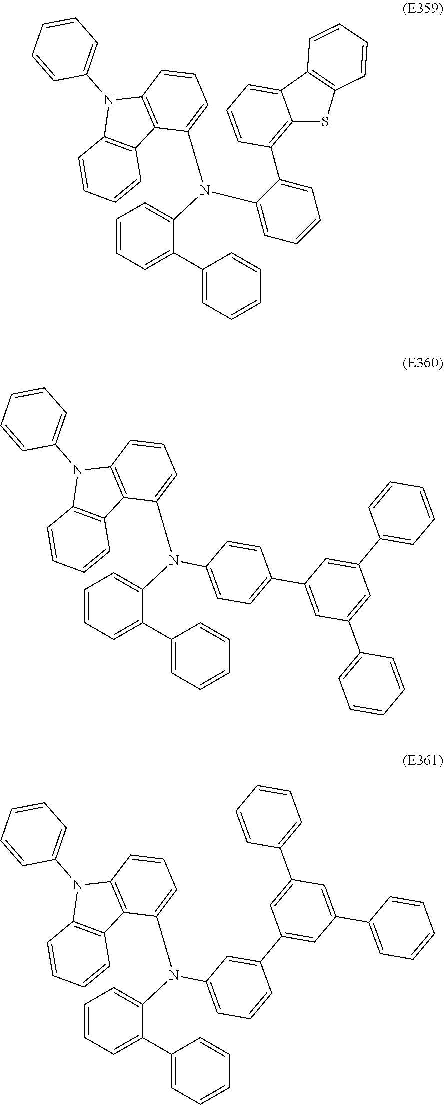 [SMILES: c1ccc(-c2ccccc2N(c2ccccc2-c2cccc3c2sc2ccccc23)c2cccc3c2c2ccccc2n3-c2ccccc2)cc1 ; c1ccc(-c2cc(-c3ccccc3)cc(-c3ccc(N(c4ccccc4-c4ccccc4)c4cccc5c4c4ccccc4n5-c4ccccc4)cc3)c2)cc1 ; c1ccc(-c2cc(-c3ccccc3)cc(-c3cccc(N(c4ccccc4-c4ccccc4)c4cccc5c4c4ccccc4n5-c4ccccc4)c3)c2)cc1]

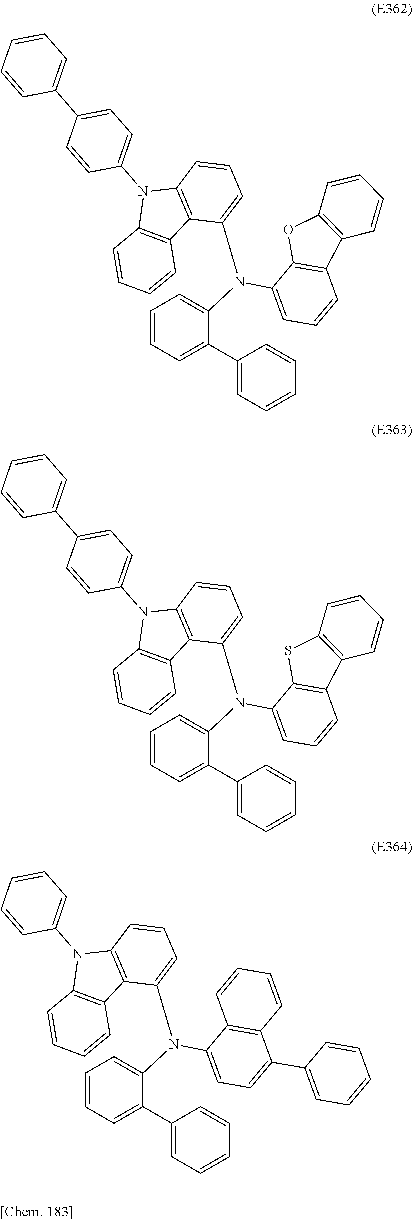 [SMILES: c1ccc(-c2ccc(-n3c4ccccc4c4c(N(c5ccccc5-c5ccccc5)c5cccc6c5oc5ccccc56)cccc43)cc2)cc1 ; c1ccc(-c2ccc(-n3c4ccccc4c4c(N(c5ccccc5-c5ccccc5)c5cccc6c5sc5ccccc56)cccc43)cc2)cc1 ; c1ccc(-c2ccccc2N(c2ccc(-c3ccccc3)c3ccccc23)c2cccc3c2c2ccccc2n3-c2ccccc2)cc1]

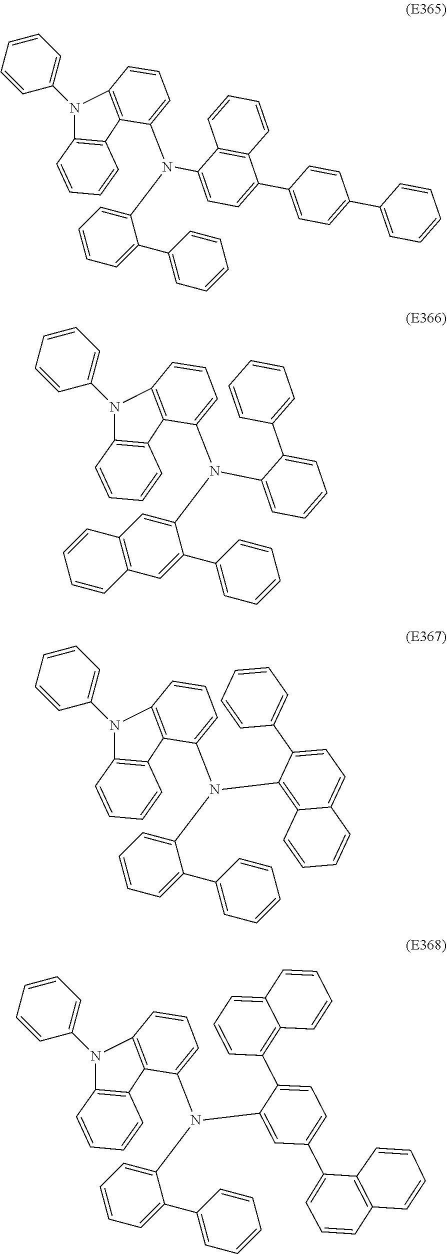 [SMILES: c1ccc(-c2ccc(-c3ccc(N(c4ccccc4-c4ccccc4)c4cccc5c4c4ccccc4n5-c4ccccc4)c4ccccc34)cc2)cc1 ; c1ccc(-c2ccccc2N(c2cc3ccccc3cc2-c2ccccc2)c2cccc3c2c2ccccc2n3-c2ccccc2)cc1 ; c1ccc(-c2ccccc2N(c2c(-c3ccccc3)ccc3ccccc23)c2cccc3c2c2ccccc2n3-c2ccccc2)cc1 ; c1ccc(-c2ccccc2N(c2cc(-c3cccc4ccccc34)ccc2-c2cccc3ccccc23)c2cccc3c2c2ccccc2n3-c2ccccc2)cc1]

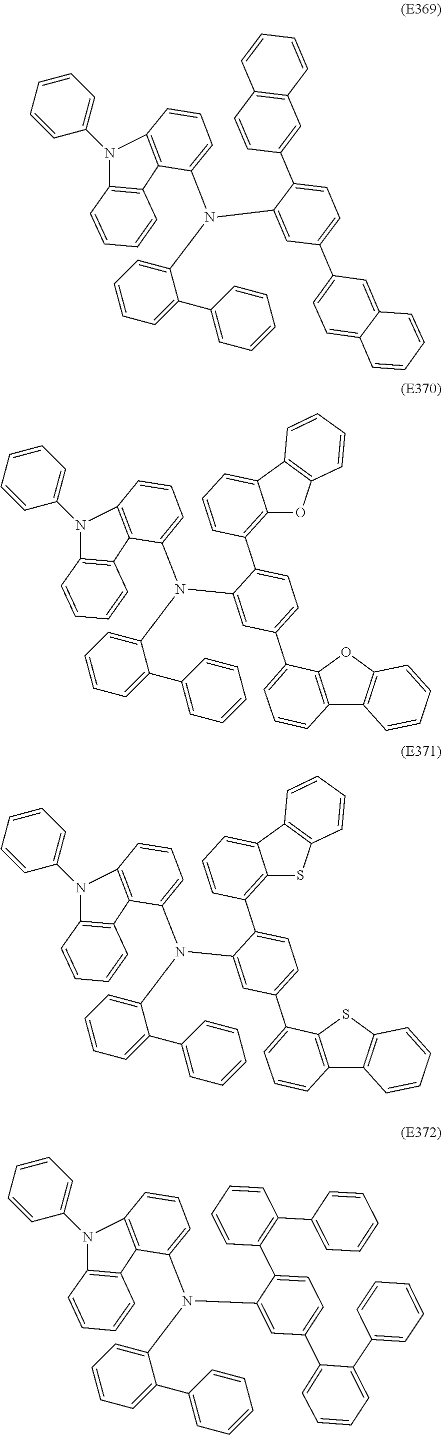 [SMILES: c1ccc(-c2ccccc2N(c2cc(-c3ccc4ccccc4c3)ccc2-c2ccc3ccccc3c2)c2cccc3c2c2ccccc2n3-c2ccccc2)cc1 ; c1ccc(-c2ccccc2N(c2cc(-c3cccc4c3oc3ccccc34)ccc2-c2cccc3c2oc2ccccc23)c2cccc3c2c2ccccc2n3-c2ccccc2)cc1 ; c1ccc(-c2ccccc2N(c2cc(-c3cccc4c3sc3ccccc34)ccc2-c2cccc3c2sc2ccccc23)c2cccc3c2c2ccccc2n3-c2ccccc2)cc1 ; c1ccc(-c2ccccc2-c2ccc(-c3ccccc3-c3ccccc3)c(N(c3ccccc3-c3ccccc3)c3cccc4c3c3ccccc3n4-c3ccccc3)c2)cc1]

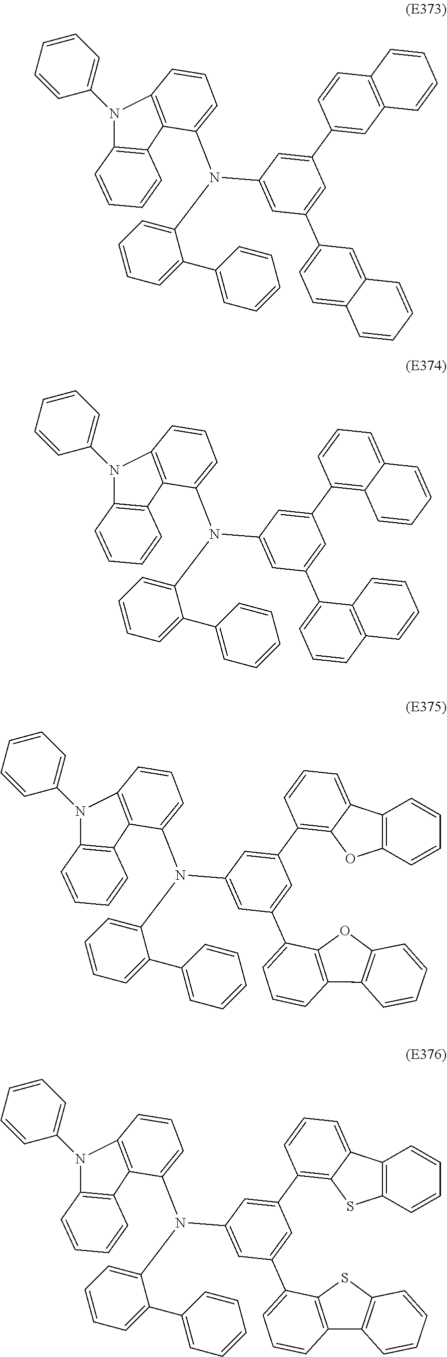 [SMILES: c1ccc(-c2ccccc2N(c2cc(-c3ccc4ccccc4c3)cc(-c3ccc4ccccc4c3)c2)c2cccc3c2c2ccccc2n3-c2ccccc2)cc1 ; c1ccc(-c2ccccc2N(c2cc(-c3cccc4ccccc34)cc(-c3cccc4ccccc34)c2)c2cccc3c2c2ccccc2n3-c2ccccc2)cc1 ; c1ccc(-c2ccccc2N(c2cc(-c3cccc4c3oc3ccccc34)cc(-c3cccc4c3oc3ccccc34)c2)c2cccc3c2c2ccccc2n3-c2ccccc2)cc1 ; c1ccc(-c2ccccc2N(c2cc(-c3cccc4c3sc3ccccc34)cc(-c3cccc4c3sc3ccccc34)c2)c2cccc3c2c2ccccc2n3-c2ccccc2)cc1]

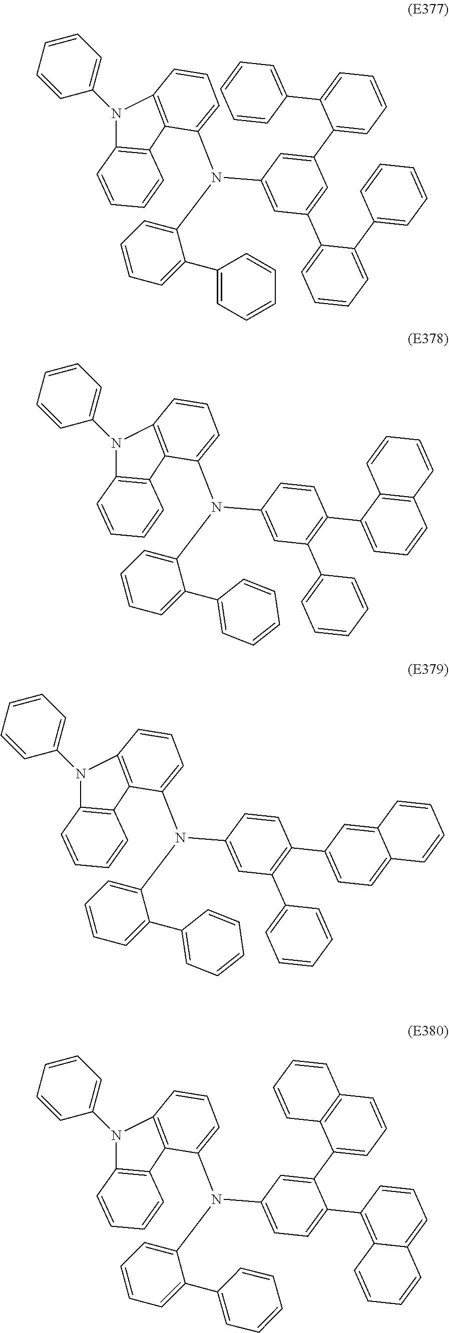 [SMILES: c1ccc(-c2ccccc2-c2cc(-c3ccccc3-c3ccccc3)cc(N(c3ccccc3-c3ccccc3)c3cccc4c3c3ccccc3n4-c3ccccc3)c2)cc1 ; c1ccc(-c2cc(N(c3ccccc3-c3ccccc3)c3cccc4c3c3ccccc3n4-c3ccccc3)ccc2-c2cccc3ccccc23)cc1 ; c1ccc(-c2cc(N(c3ccccc3-c3ccccc3)c3cccc4c3c3ccccc3n4-c3ccccc3)ccc2-c2ccc3ccccc3c2)cc1 ; c1ccc(-c2ccccc2N(c2ccc(-c3cccc4ccccc34)c(-c3cccc4ccccc34)c2)c2cccc3c2c2ccccc2n3-c2ccccc2)cc1]

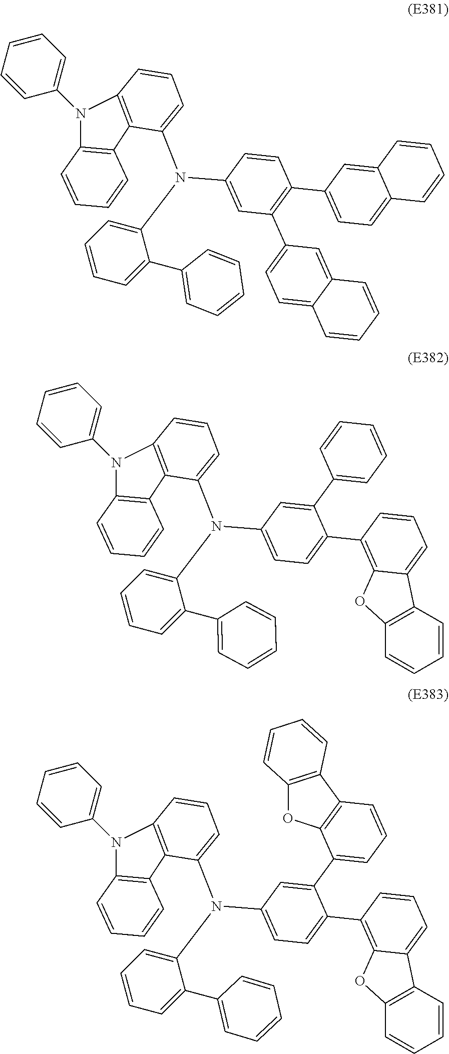 [SMILES: c1ccc(-c2ccccc2N(c2ccc(-c3ccc4ccccc4c3)c(-c3ccc4ccccc4c3)c2)c2cccc3c2c2ccccc2n3-c2ccccc2)cc1 ; c1ccc(-c2cc(N(c3ccccc3-c3ccccc3)c3cccc4c3c3ccccc3n4-c3ccccc3)ccc2-c2cccc3c2oc2ccccc23)cc1 ; c1ccc(-c2ccccc2N(c2ccc(-c3cccc4c3oc3ccccc34)c(-c3cccc4c3oc3ccccc34)c2)c2cccc3c2c2ccccc2n3-c2ccccc2)cc1]

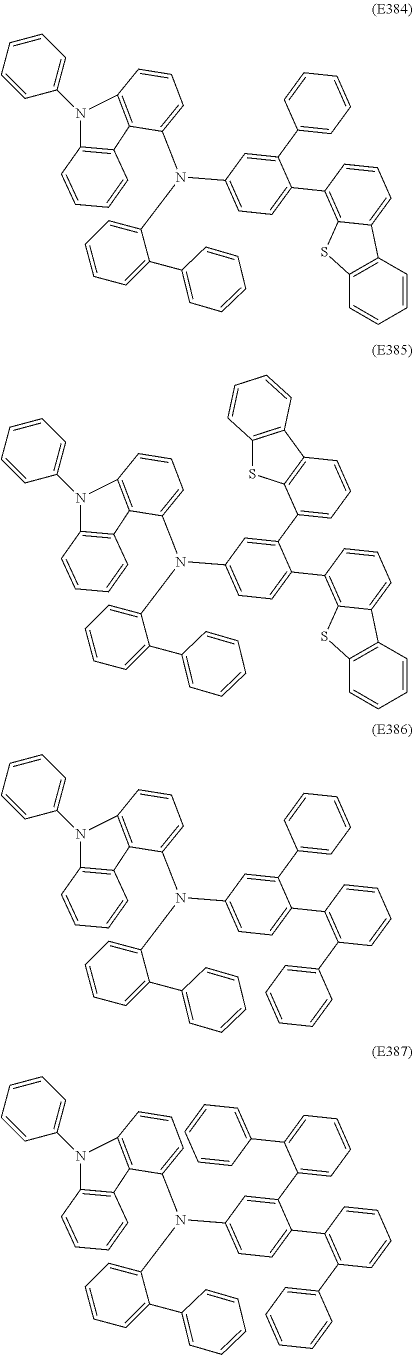 [SMILES: c1ccc(-c2cc(N(c3ccccc3-c3ccccc3)c3cccc4c3c3ccccc3n4-c3ccccc3)ccc2-c2cccc3c2sc2ccccc23)cc1 ; c1ccc(-c2ccccc2N(c2ccc(-c3cccc4c3sc3ccccc34)c(-c3cccc4c3sc3ccccc34)c2)c2cccc3c2c2ccccc2n3-c2ccccc2)cc1 ; c1ccc(-c2ccccc2-c2ccc(N(c3ccccc3-c3ccccc3)c3cccc4c3c3ccccc3n4-c3ccccc3)cc2-c2ccccc2)cc1 ; c1ccc(-c2ccccc2-c2ccc(N(c3ccccc3-c3ccccc3)c3cccc4c3c3ccccc3n4-c3ccccc3)cc2-c2ccccc2-c2ccccc2)cc1]

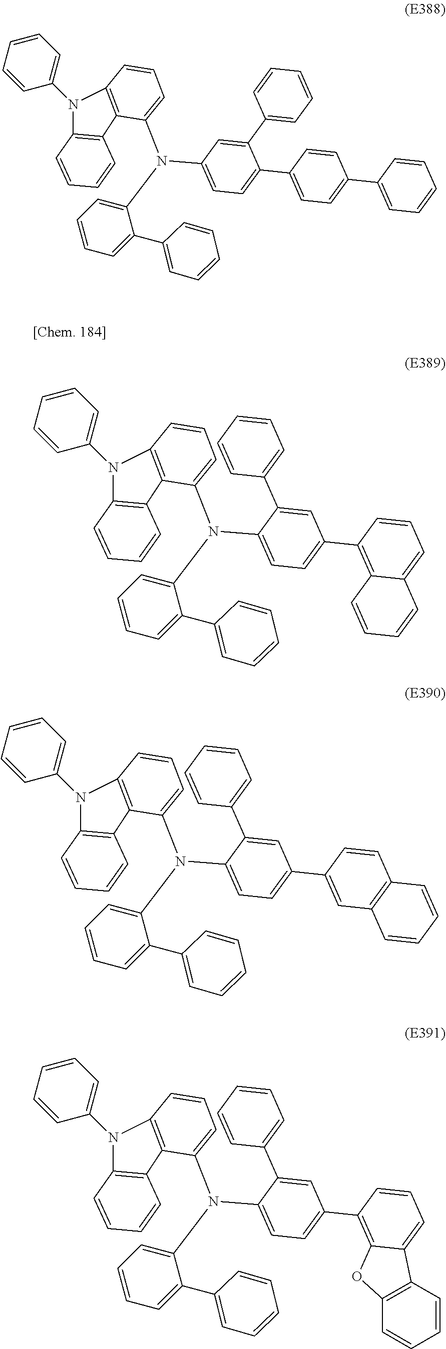 [SMILES: c1ccc(-c2ccc(-c3ccc(N(c4ccccc4-c4ccccc4)c4cccc5c4c4ccccc4n5-c4ccccc4)cc3-c3ccccc3)cc2)cc1 ; c1ccc(-c2ccccc2N(c2ccc(-c3cccc4ccccc34)cc2-c2ccccc2)c2cccc3c2c2ccccc2n3-c2ccccc2)cc1 ; c1ccc(-c2ccccc2N(c2ccc(-c3ccc4ccccc4c3)cc2-c2ccccc2)c2cccc3c2c2ccccc2n3-c2ccccc2)cc1 ; c1ccc(-c2ccccc2N(c2ccc(-c3cccc4c3oc3ccccc34)cc2-c2ccccc2)c2cccc3c2c2ccccc2n3-c2ccccc2)cc1]

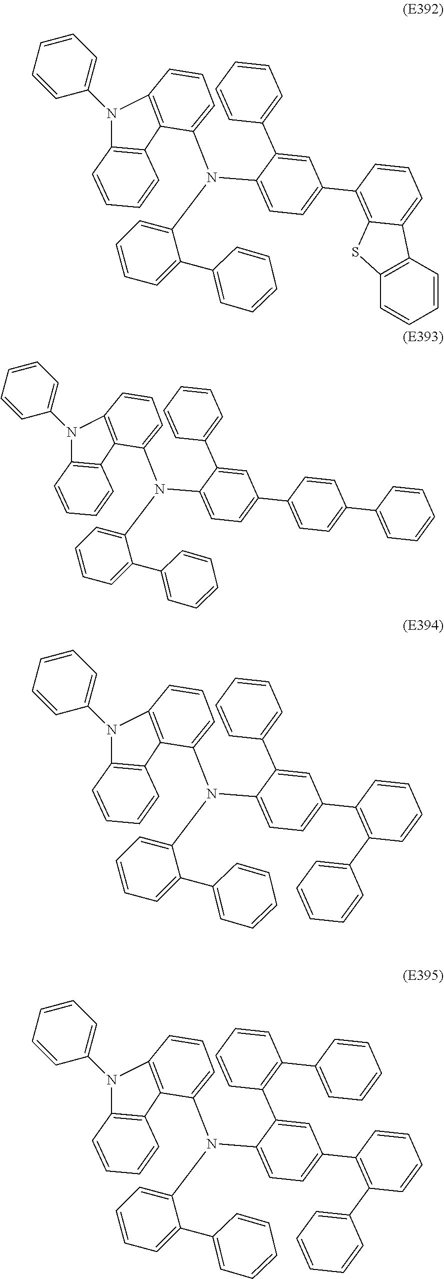 [SMILES: c1ccc(-c2ccccc2N(c2ccc(-c3cccc4c3sc3ccccc34)cc2-c2ccccc2)c2cccc3c2c2ccccc2n3-c2ccccc2)cc1 ; c1ccc(-c2ccc(-c3ccc(N(c4ccccc4-c4ccccc4)c4cccc5c4c4ccccc4n5-c4ccccc4)c(-c4ccccc4)c3)cc2)cc1 ; c1ccc(-c2ccccc2-c2ccc(N(c3ccccc3-c3ccccc3)c3cccc4c3c3ccccc3n4-c3ccccc3)c(-c3ccccc3)c2)cc1 ; c1ccc(-c2ccccc2-c2ccc(N(c3ccccc3-c3ccccc3)c3cccc4c3c3ccccc3n4-c3ccccc3)c(-c3ccccc3-c3ccccc3)c2)cc1]

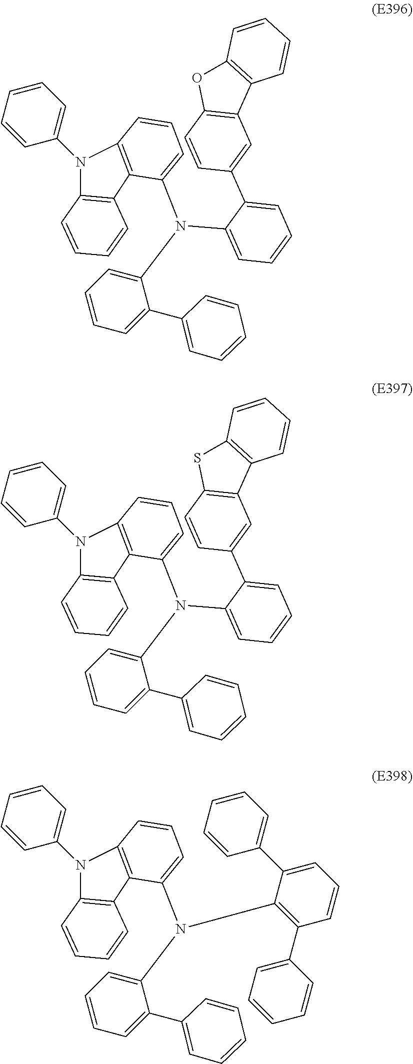 [SMILES: c1ccc(-c2ccccc2N(c2ccccc2-c2ccc3oc4ccccc4c3c2)c2cccc3c2c2ccccc2n3-c2ccccc2)cc1 ; c1ccc(-c2ccccc2N(c2ccccc2-c2ccc3sc4ccccc4c3c2)c2cccc3c2c2ccccc2n3-c2ccccc2)cc1 ; c1ccc(-c2ccccc2N(c2c(-c3ccccc3)cccc2-c2ccccc2)c2cccc3c2c2ccccc2n3-c2ccccc2)cc1]

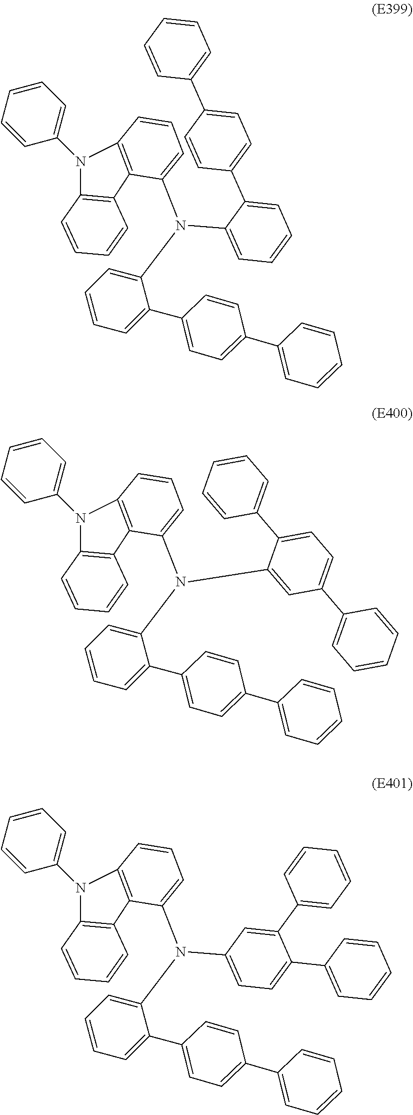 [SMILES: c1ccc(-c2ccc(-c3ccccc3N(c3ccccc3-c3ccc(-c4ccccc4)cc3)c3cccc4c3c3ccccc3n4-c3ccccc3)cc2)cc1 ; c1ccc(-c2ccc(-c3ccccc3N(c3cc(-c4ccccc4)ccc3-c3ccccc3)c3cccc4c3c3ccccc3n4-c3ccccc3)cc2)cc1 ; c1ccc(-c2ccc(-c3ccccc3N(c3ccc(-c4ccccc4)c(-c4ccccc4)c3)c3cccc4c3c3ccccc3n4-c3ccccc3)cc2)cc1]

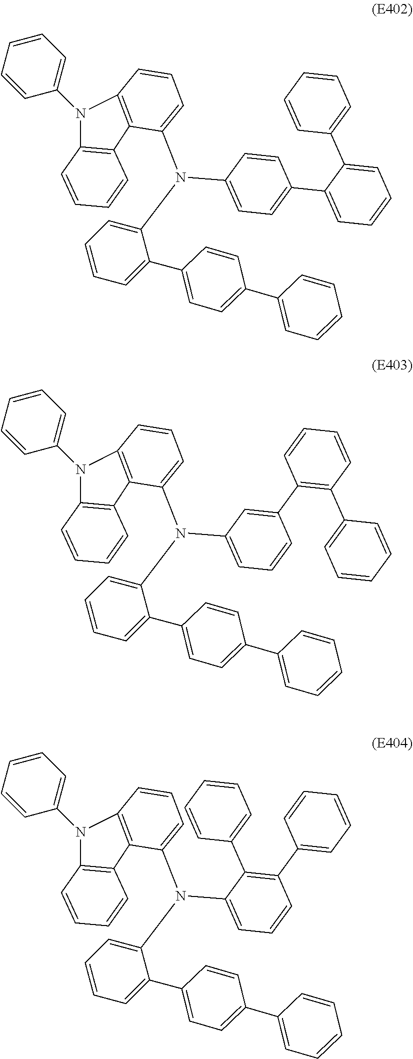 [SMILES: c1ccc(-c2ccc(-c3ccccc3N(c3ccc(-c4ccccc4-c4ccccc4)cc3)c3cccc4c3c3ccccc3n4-c3ccccc3)cc2)cc1 ; c1ccc(-c2ccc(-c3ccccc3N(c3cccc(-c4ccccc4-c4ccccc4)c3)c3cccc4c3c3ccccc3n4-c3ccccc3)cc2)cc1 ; c1ccc(-c2ccc(-c3ccccc3N(c3cccc(-c4ccccc4)c3-c3ccccc3)c3cccc4c3c3ccccc3n4-c3ccccc3)cc2)cc1]

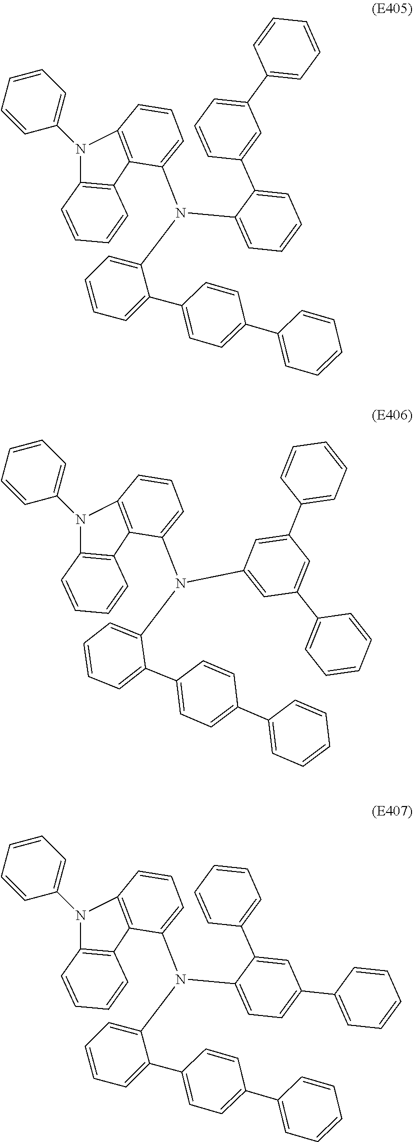 [SMILES: c1ccc(-c2ccc(-c3ccccc3N(c3ccccc3-c3cccc(-c4ccccc4)c3)c3cccc4c3c3ccccc3n4-c3ccccc3)cc2)cc1 ; c1ccc(-c2ccc(-c3ccccc3N(c3cc(-c4ccccc4)cc(-c4ccccc4)c3)c3cccc4c3c3ccccc3n4-c3ccccc3)cc2)cc1 ; c1ccc(-c2ccc(-c3ccccc3N(c3ccc(-c4ccccc4)cc3-c3ccccc3)c3cccc4c3c3ccccc3n4-c3ccccc3)cc2)cc1]

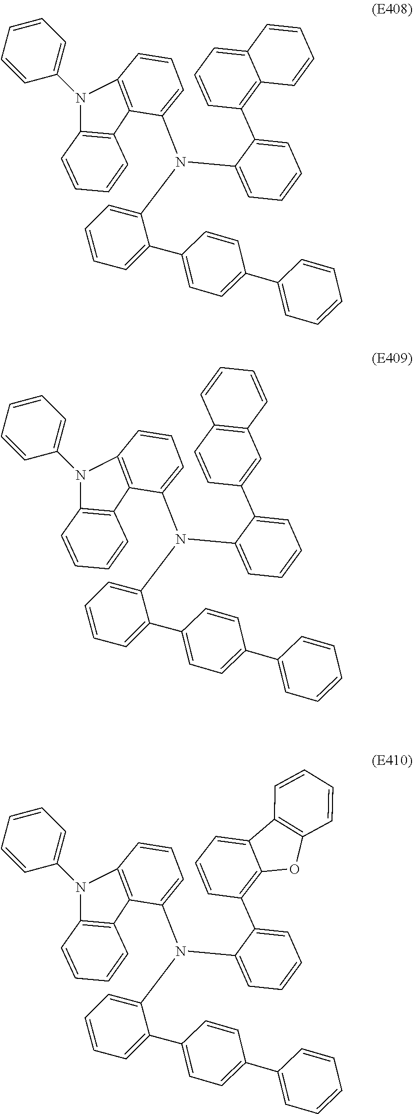 [SMILES: c1ccc(-c2ccc(-c3ccccc3N(c3ccccc3-c3cccc4ccccc34)c3cccc4c3c3ccccc3n4-c3ccccc3)cc2)cc1 ; c1ccc(-c2ccc(-c3ccccc3N(c3ccccc3-c3ccc4ccccc4c3)c3cccc4c3c3ccccc3n4-c3ccccc3)cc2)cc1 ; c1ccc(-c2ccc(-c3ccccc3N(c3ccccc3-c3cccc4c3oc3ccccc34)c3cccc4c3c3ccccc3n4-c3ccccc3)cc2)cc1]

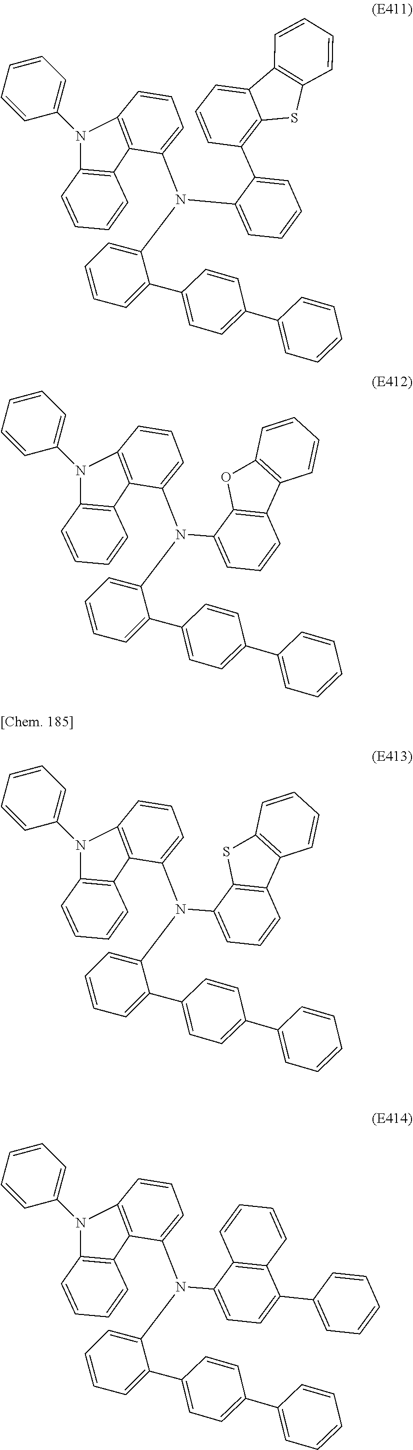 [SMILES: c1ccc(-c2ccc(-c3ccccc3N(c3ccccc3-c3cccc4c3sc3ccccc34)c3cccc4c3c3ccccc3n4-c3ccccc3)cc2)cc1 ; c1ccc(-c2ccc(-c3ccccc3N(c3cccc4c3oc3ccccc34)c3cccc4c3c3ccccc3n4-c3ccccc3)cc2)cc1 ; c1ccc(-c2ccc(-c3ccccc3N(c3cccc4c3sc3ccccc34)c3cccc4c3c3ccccc3n4-c3ccccc3)cc2)cc1 ; c1ccc(-c2ccc(-c3ccccc3N(c3ccc(-c4ccccc4)c4ccccc34)c3cccc4c3c3ccccc3n4-c3ccccc3)cc2)cc1]

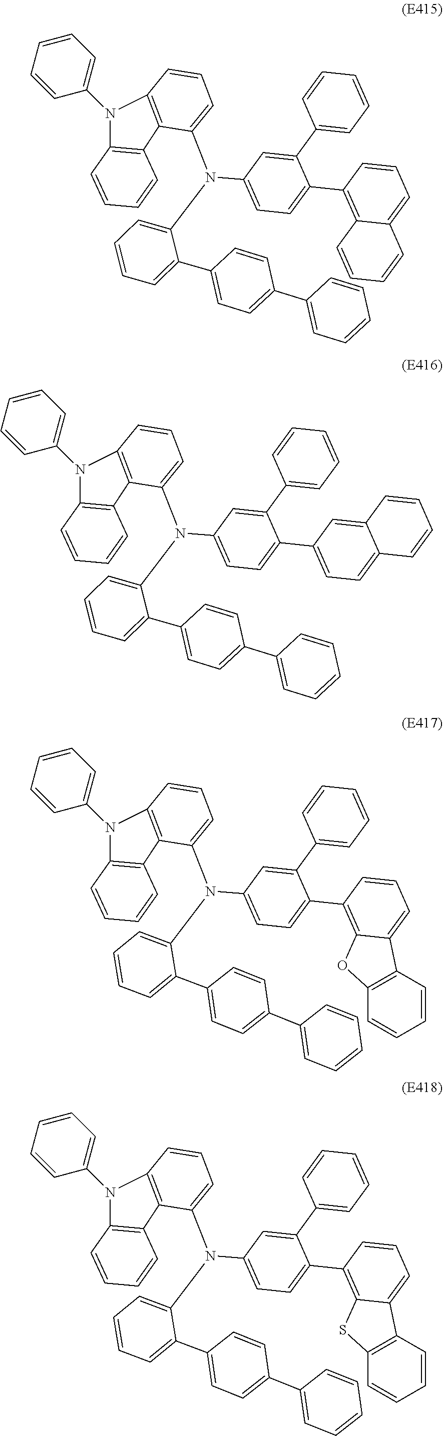 [SMILES: c1ccc(-c2ccc(-c3ccccc3N(c3ccc(-c4cccc5ccccc45)c(-c4ccccc4)c3)c3cccc4c3c3ccccc3n4-c3ccccc3)cc2)cc1 ; c1ccc(-c2ccc(-c3ccccc3N(c3ccc(-c4ccc5ccccc5c4)c(-c4ccccc4)c3)c3cccc4c3c3ccccc3n4-c3ccccc3)cc2)cc1 ; c1ccc(-c2ccc(-c3ccccc3N(c3ccc(-c4cccc5c4oc4ccccc45)c(-c4ccccc4)c3)c3cccc4c3c3ccccc3n4-c3ccccc3)cc2)cc1 ; c1ccc(-c2ccc(-c3ccccc3N(c3ccc(-c4cccc5c4sc4ccccc45)c(-c4ccccc4)c3)c3cccc4c3c3ccccc3n4-c3ccccc3)cc2)cc1]

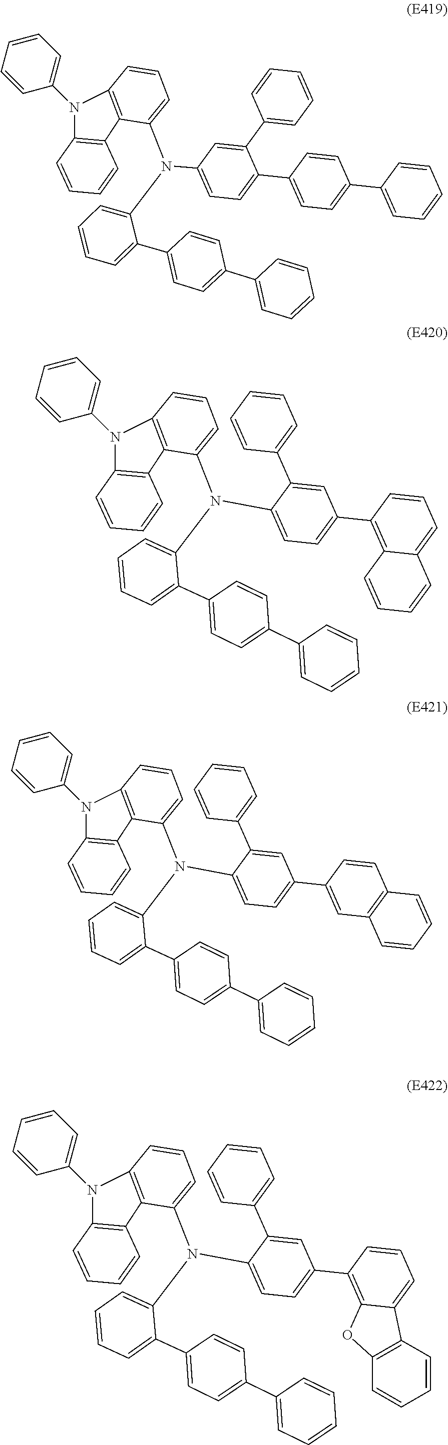 [SMILES: c1ccc(-c2ccc(-c3ccc(N(c4ccccc4-c4ccc(-c5ccccc5)cc4)c4cccc5c4c4ccccc4n5-c4ccccc4)cc3-c3ccccc3)cc2)cc1 ; c1ccc(-c2ccc(-c3ccccc3N(c3ccc(-c4cccc5ccccc45)cc3-c3ccccc3)c3cccc4c3c3ccccc3n4-c3ccccc3)cc2)cc1 ; c1ccc(-c2ccc(-c3ccccc3N(c3ccc(-c4ccc5ccccc5c4)cc3-c3ccccc3)c3cccc4c3c3ccccc3n4-c3ccccc3)cc2)cc1 ; c1ccc(-c2ccc(-c3ccccc3N(c3ccc(-c4cccc5c4oc4ccccc45)cc3-c3ccccc3)c3cccc4c3c3ccccc3n4-c3ccccc3)cc2)cc1]

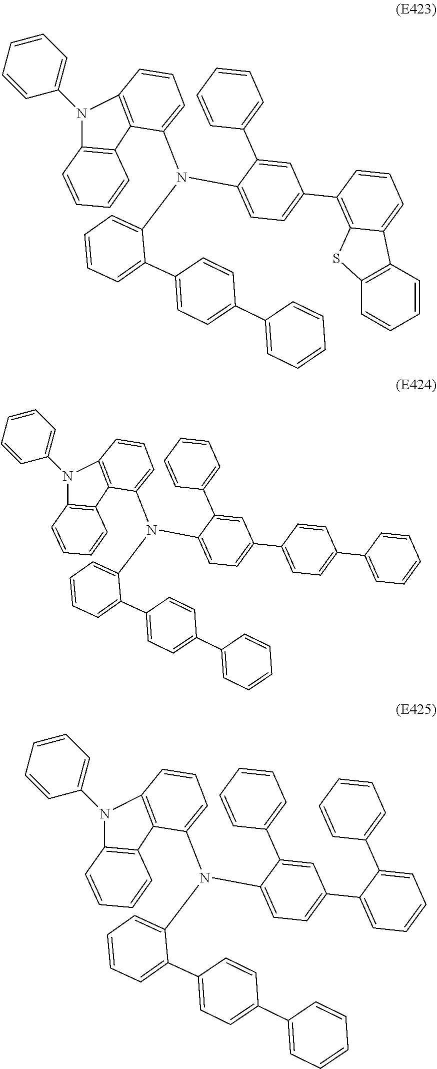 [SMILES: c1ccc(-c2ccc(-c3ccccc3N(c3ccc(-c4cccc5c4sc4ccccc45)cc3-c3ccccc3)c3cccc4c3c3ccccc3n4-c3ccccc3)cc2)cc1 ; c1ccc(-c2ccc(-c3ccc(N(c4ccccc4-c4ccc(-c5ccccc5)cc4)c4cccc5c4c4ccccc4n5-c4ccccc4)c(-c4ccccc4)c3)cc2)cc1 ; c1ccc(-c2ccc(-c3ccccc3N(c3ccc(-c4ccccc4-c4ccccc4)cc3-c3ccccc3)c3cccc4c3c3ccccc3n4-c3ccccc3)cc2)cc1]

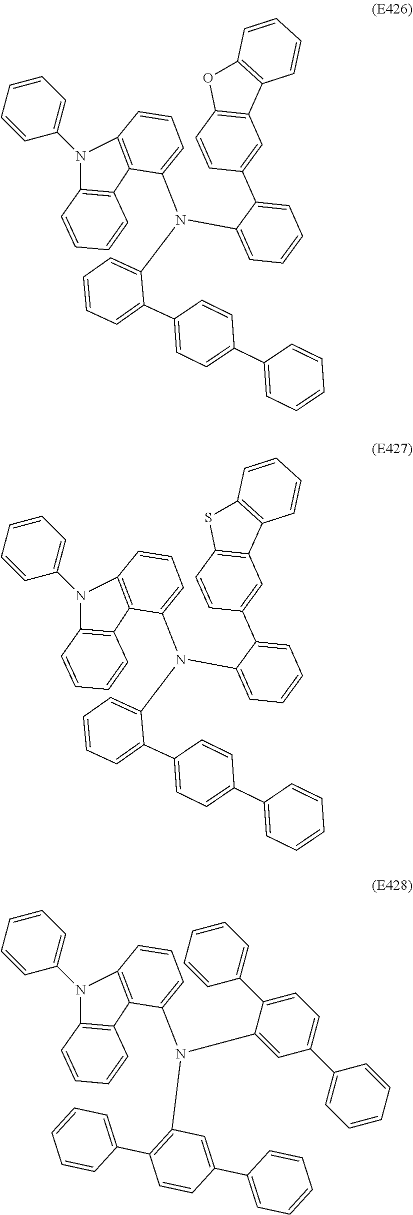 [SMILES: c1ccc(-c2ccc(-c3ccccc3N(c3ccccc3-c3ccc4oc5ccccc5c4c3)c3cccc4c3c3ccccc3n4-c3ccccc3)cc2)cc1 ; c1ccc(-c2ccc(-c3ccccc3N(c3ccccc3-c3ccc4sc5ccccc5c4c3)c3cccc4c3c3ccccc3n4-c3ccccc3)cc2)cc1 ; c1ccc(-c2ccc(-c3ccccc3)c(N(c3cc(-c4ccccc4)ccc3-c3ccccc3)c3cccc4c3c3ccccc3n4-c3ccccc3)c2)cc1]

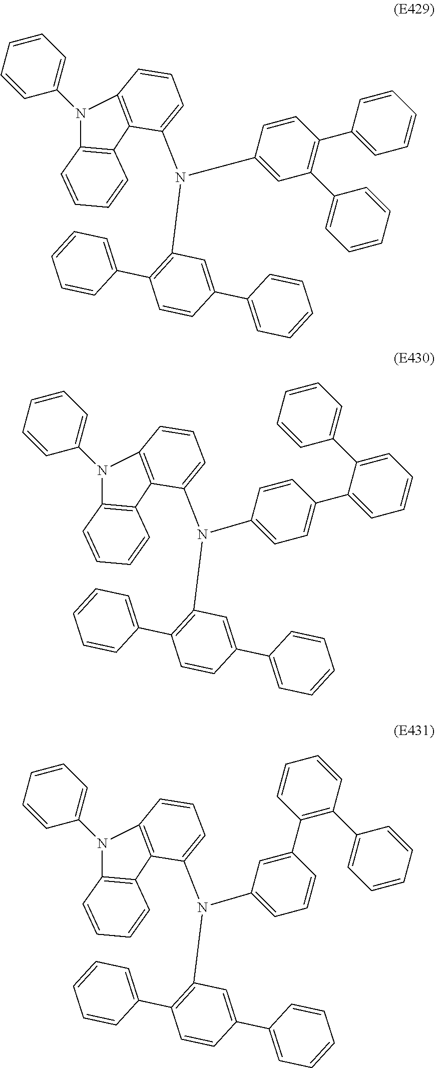 [SMILES: c1ccc(-c2ccc(-c3ccccc3)c(N(c3ccc(-c4ccccc4)c(-c4ccccc4)c3)c3cccc4c3c3ccccc3n4-c3ccccc3)c2)cc1 ; c1ccc(-c2ccc(-c3ccccc3)c(N(c3ccc(-c4ccccc4-c4ccccc4)cc3)c3cccc4c3c3ccccc3n4-c3ccccc3)c2)cc1 ; c1ccc(-c2ccc(-c3ccccc3)c(N(c3cccc(-c4ccccc4-c4ccccc4)c3)c3cccc4c3c3ccccc3n4-c3ccccc3)c2)cc1]

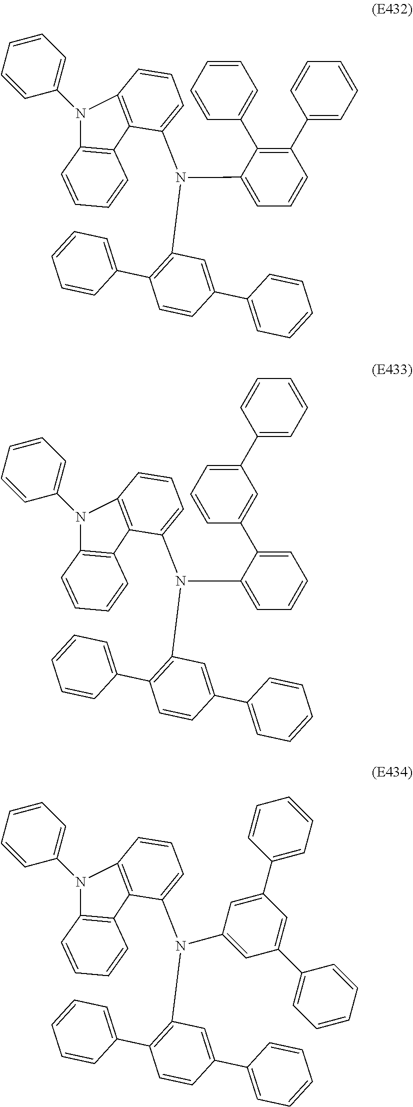

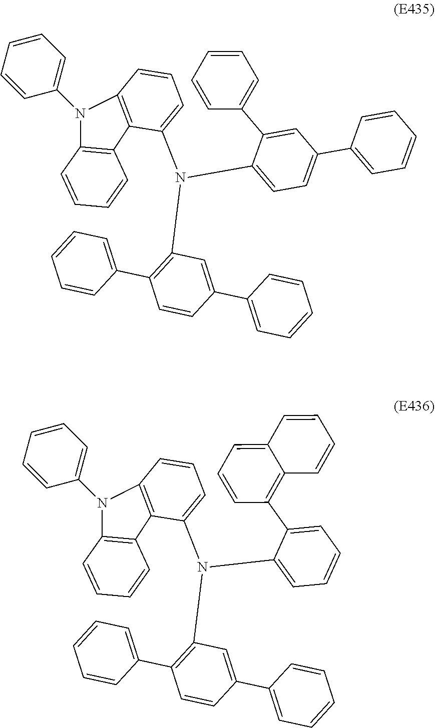 [SMILES: c1ccc(-c2ccc(N(c3cc(-c4ccccc4)ccc3-c3ccccc3)c3cccc4c3c3ccccc3n4-c3ccccc3)c(-c3ccccc3)c2)cc1 ; c1ccc(-c2ccc(-c3ccccc3)c(N(c3ccccc3-c3cccc4ccccc34)c3cccc4c3c3ccccc3n4-c3ccccc3)c2)cc1]

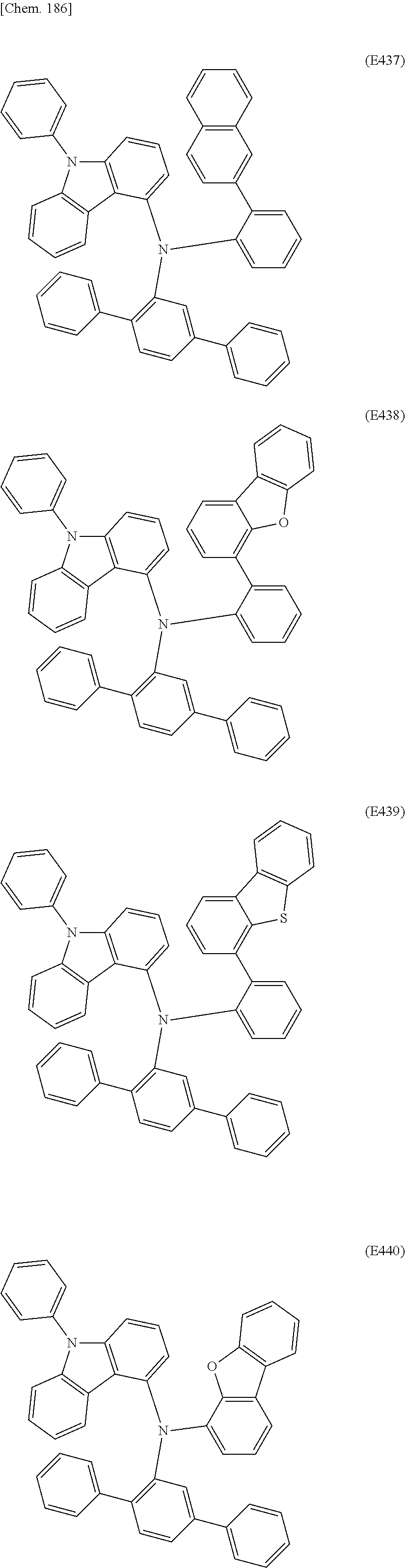

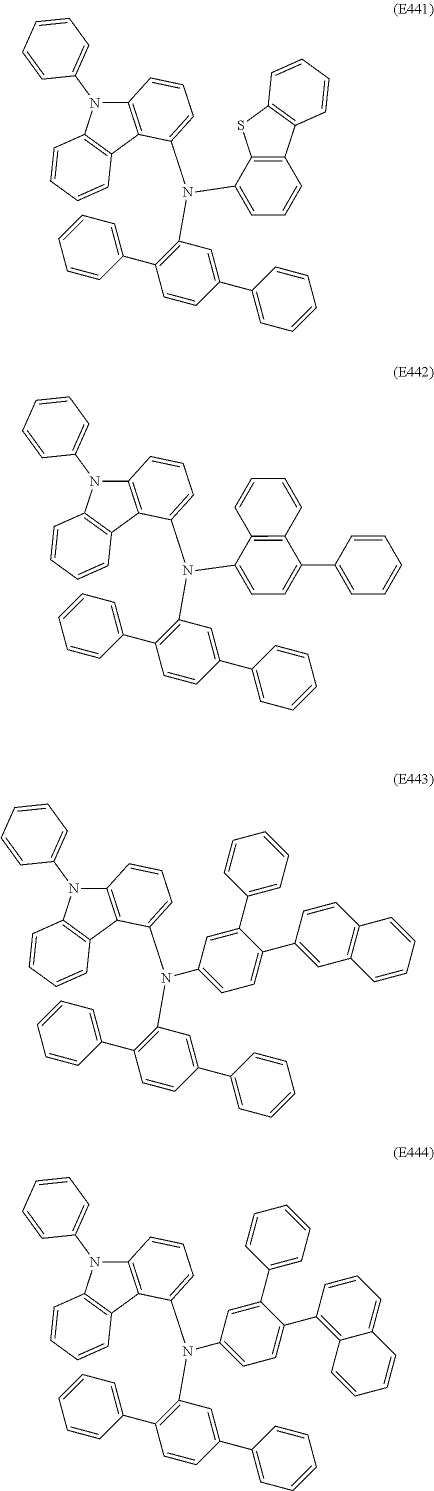 [SMILES: c1ccc(-c2ccc(-c3ccccc3)c(N(c3cccc4c3sc3ccccc34)c3cccc4c3c3ccccc3n4-c3ccccc3)c2)cc1 ; c1ccc(-c2ccc(-c3ccccc3)c(N(c3ccc(-c4ccccc4)c4ccccc34)c3cccc4c3c3ccccc3n4-c3ccccc3)c2)cc1 ; c1ccc(-c2ccc(-c3ccccc3)c(N(c3ccc(-c4ccc5ccccc5c4)c(-c4ccccc4)c3)c3cccc4c3c3ccccc3n4-c3ccccc3)c2)cc1 ; c1ccc(-c2ccc(-c3ccccc3)c(N(c3ccc(-c4cccc5ccccc45)c(-c4ccccc4)c3)c3cccc4c3c3ccccc3n4-c3ccccc3)c2)cc1]

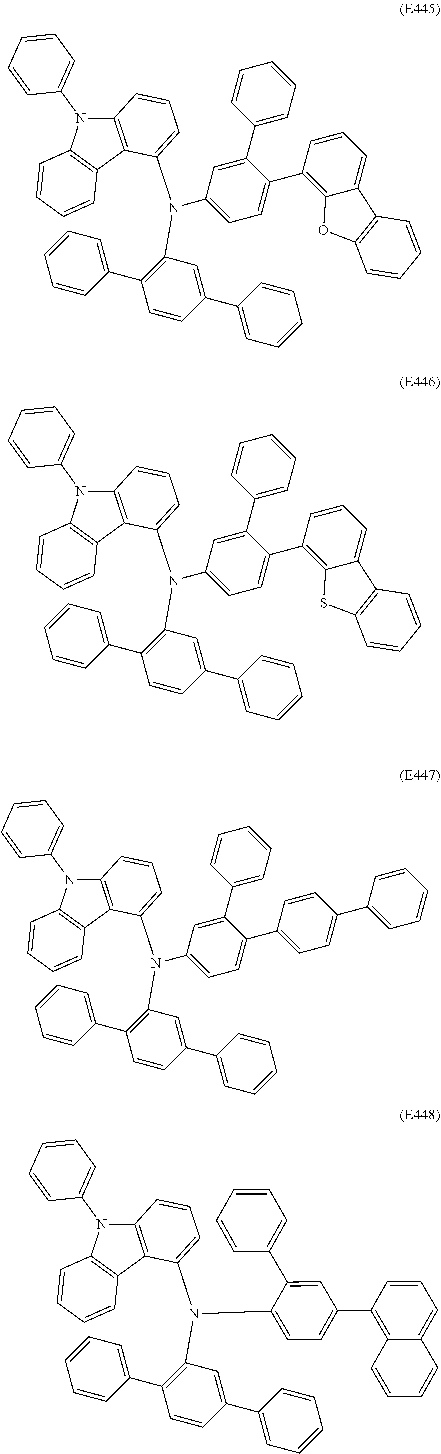 [SMILES: c1ccc(-c2ccc(-c3ccccc3)c(N(c3ccc(-c4cccc5c4oc4ccccc45)c(-c4ccccc4)c3)c3cccc4c3c3ccccc3n4-c3ccccc3)c2)cc1 ; c1ccc(-c2ccc(-c3ccccc3)c(N(c3ccc(-c4cccc5c4sc4ccccc45)c(-c4ccccc4)c3)c3cccc4c3c3ccccc3n4-c3ccccc3)c2)cc1 ; c1ccc(-c2ccc(-c3ccc(N(c4cc(-c5ccccc5)ccc4-c4ccccc4)c4cccc5c4c4ccccc4n5-c4ccccc4)cc3-c3ccccc3)cc2)cc1 ; c1ccc(-c2ccc(-c3ccccc3)c(N(c3ccc(-c4cccc5ccccc45)cc3-c3ccccc3)c3cccc4c3c3ccccc3n4-c3ccccc3)c2)cc1]

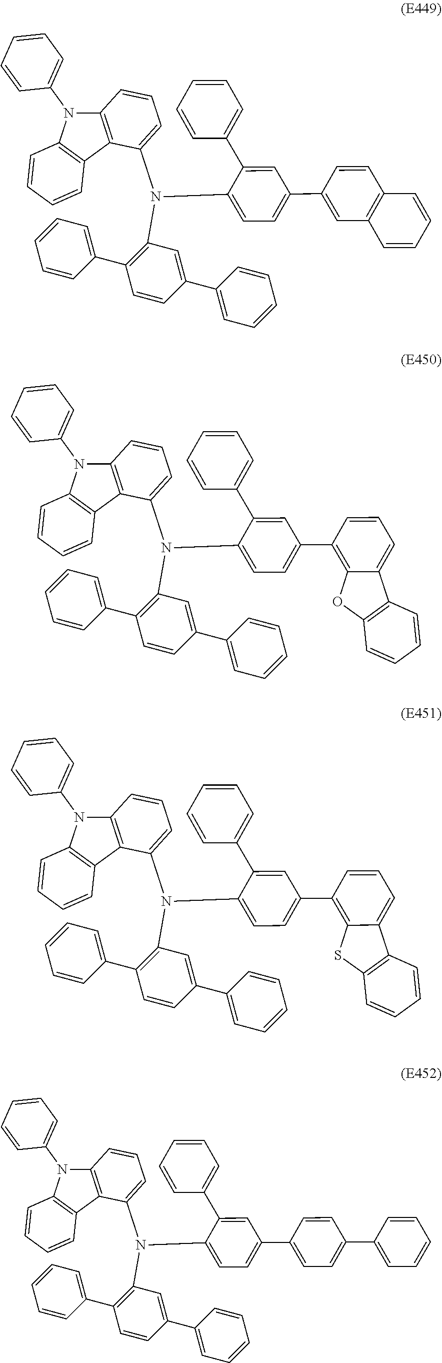 [SMILES: c1ccc(-c2ccc(-c3ccccc3)c(N(c3ccc(-c4ccc5ccccc5c4)cc3-c3ccccc3)c3cccc4c3c3ccccc3n4-c3ccccc3)c2)cc1 ; c1ccc(-c2ccc(-c3ccccc3)c(N(c3ccc(-c4cccc5c4oc4ccccc45)cc3-c3ccccc3)c3cccc4c3c3ccccc3n4-c3ccccc3)c2)cc1 ; c1ccc(-c2ccc(-c3ccccc3)c(N(c3ccc(-c4cccc5c4sc4ccccc45)cc3-c3ccccc3)c3cccc4c3c3ccccc3n4-c3ccccc3)c2)cc1 ; c1ccc(-c2ccc(-c3ccc(N(c4cc(-c5ccccc5)ccc4-c4ccccc4)c4cccc5c4c4ccccc4n5-c4ccccc4)c(-c4ccccc4)c3)cc2)cc1]

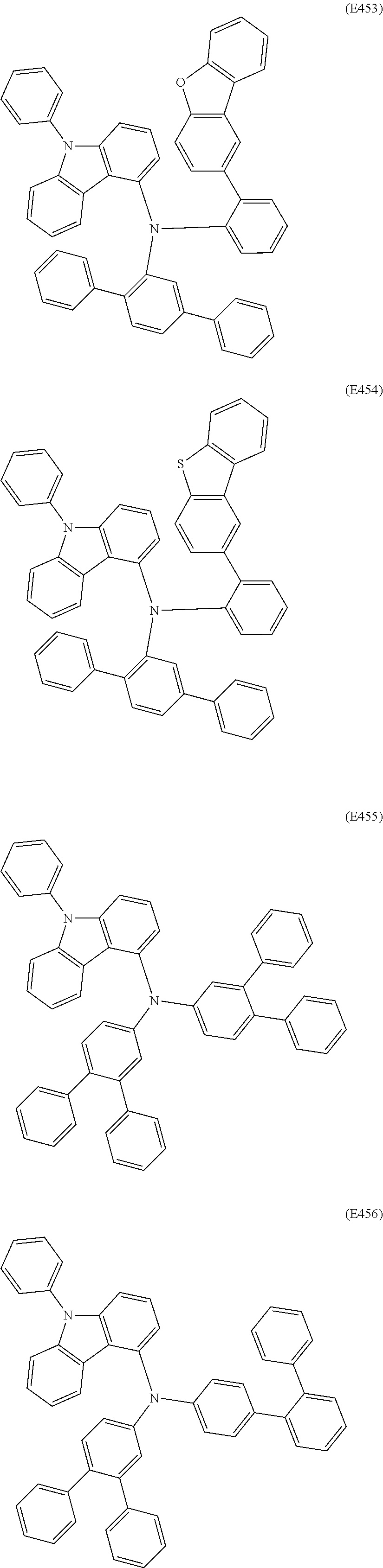

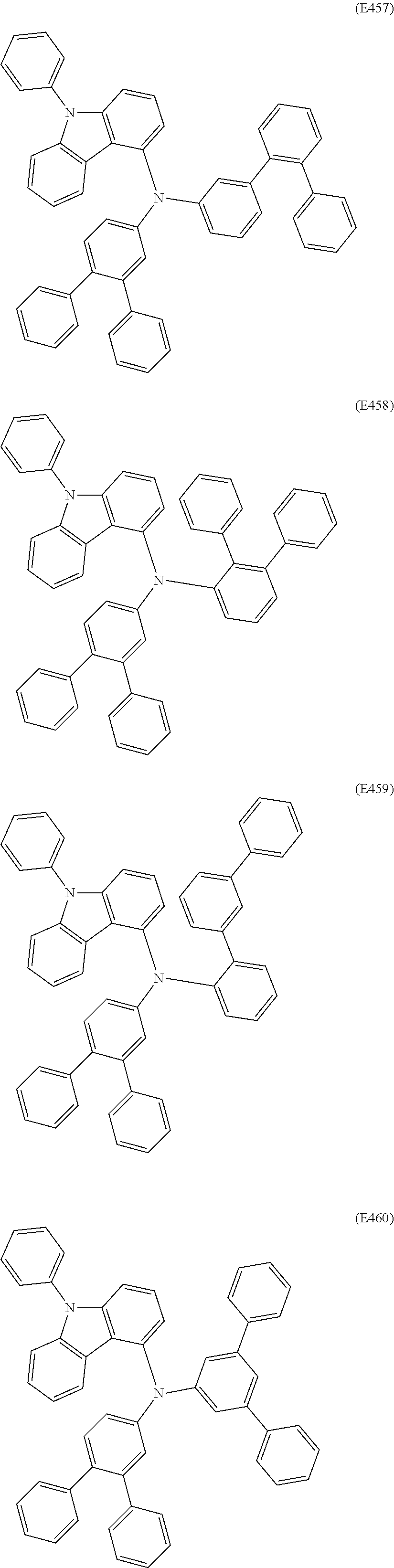 [SMILES: c1ccc(-c2ccccc2-c2cccc(N(c3ccc(-c4ccccc4)c(-c4ccccc4)c3)c3cccc4c3c3ccccc3n4-c3ccccc3)c2)cc1 ; c1ccc(-c2ccc(N(c3cccc(-c4ccccc4)c3-c3ccccc3)c3cccc4c3c3ccccc3n4-c3ccccc3)cc2-c2ccccc2)cc1 ; c1ccc(-c2cccc(-c3ccccc3N(c3ccc(-c4ccccc4)c(-c4ccccc4)c3)c3cccc4c3c3ccccc3n4-c3ccccc3)c2)cc1 ; c1ccc(-c2cc(-c3ccccc3)cc(N(c3ccc(-c4ccccc4)c(-c4ccccc4)c3)c3cccc4c3c3ccccc3n4-c3ccccc3)c2)cc1]

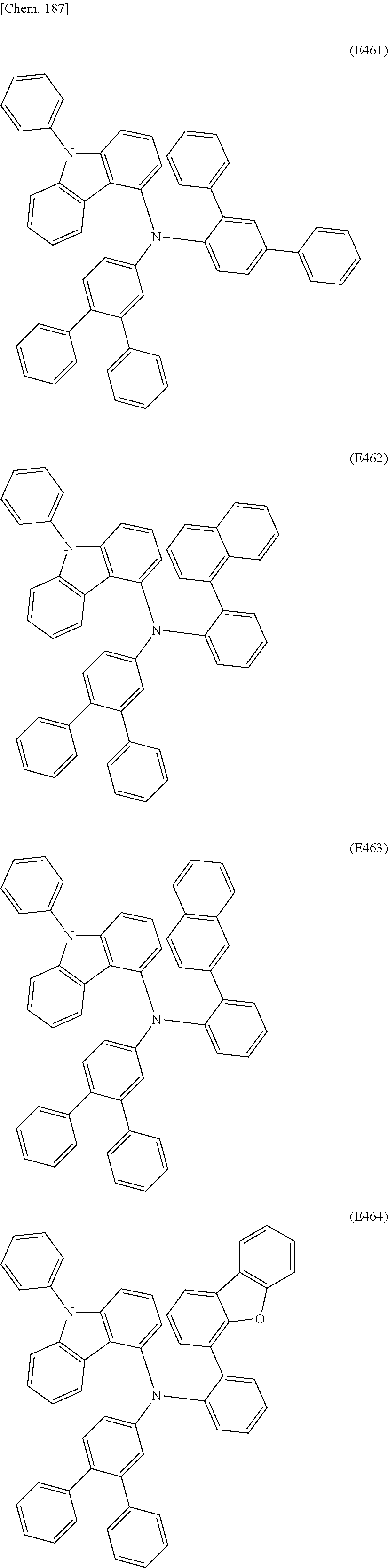 [SMILES: c1ccc(-c2ccc(N(c3ccc(-c4ccccc4)c(-c4ccccc4)c3)c3cccc4c3c3ccccc3n4-c3ccccc3)c(-c3ccccc3)c2)cc1 ; c1ccc(-c2ccc(N(c3ccccc3-c3cccc4ccccc34)c3cccc4c3c3ccccc3n4-c3ccccc3)cc2-c2ccccc2)cc1 ; c1ccc(-c2ccc(N(c3ccccc3-c3ccc4ccccc4c3)c3cccc4c3c3ccccc3n4-c3ccccc3)cc2-c2ccccc2)cc1 ; c1ccc(-c2ccc(N(c3ccccc3-c3cccc4c3oc3ccccc34)c3cccc4c3c3ccccc3n4-c3ccccc3)cc2-c2ccccc2)cc1]

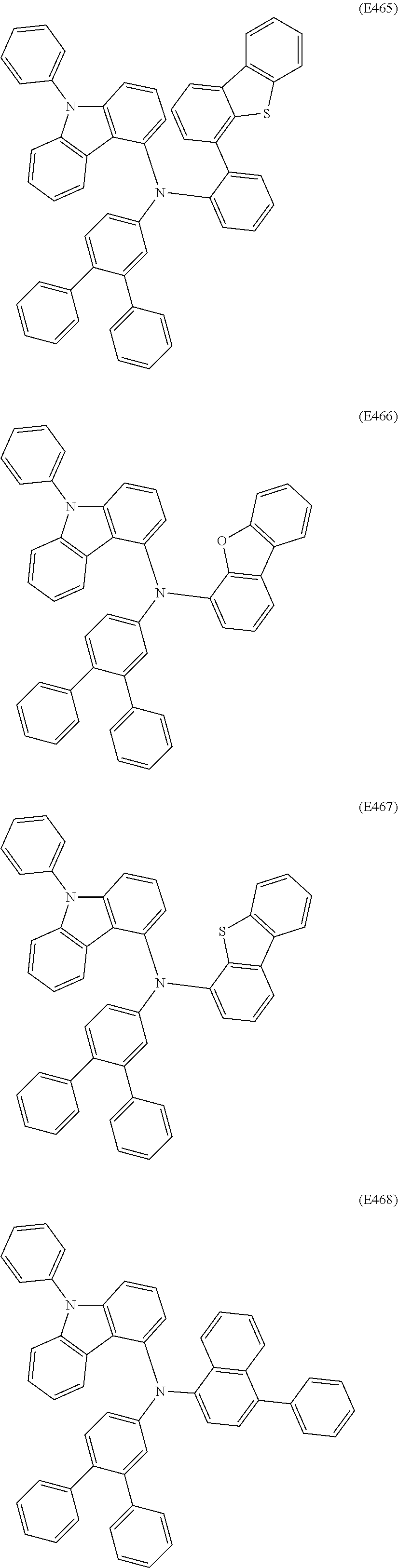 [SMILES: c1ccc(-c2ccc(N(c3ccccc3-c3cccc4c3sc3ccccc34)c3cccc4c3c3ccccc3n4-c3ccccc3)cc2-c2ccccc2)cc1 ; c1ccc(-c2ccc(N(c3cccc4c3oc3ccccc34)c3cccc4c3c3ccccc3n4-c3ccccc3)cc2-c2ccccc2)cc1 ; c1ccc(-c2ccc(N(c3cccc4c3sc3ccccc34)c3cccc4c3c3ccccc3n4-c3ccccc3)cc2-c2ccccc2)cc1 ; c1ccc(-c2ccc(N(c3ccc(-c4ccccc4)c4ccccc34)c3cccc4c3c3ccccc3n4-c3ccccc3)cc2-c2ccccc2)cc1]

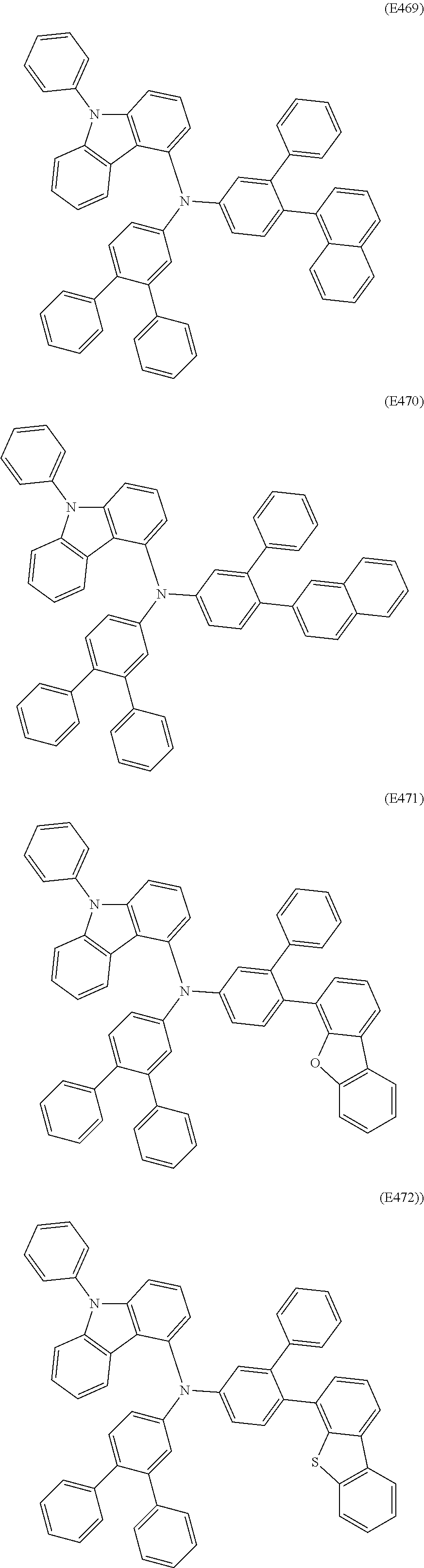 [SMILES: c1ccc(-c2ccc(N(c3ccc(-c4cccc5ccccc45)c(-c4ccccc4)c3)c3cccc4c3c3ccccc3n4-c3ccccc3)cc2-c2ccccc2)cc1 ; c1ccc(-c2ccc(N(c3ccc(-c4ccc5ccccc5c4)c(-c4ccccc4)c3)c3cccc4c3c3ccccc3n4-c3ccccc3)cc2-c2ccccc2)cc1 ; c1ccc(-c2ccc(N(c3ccc(-c4cccc5c4oc4ccccc45)c(-c4ccccc4)c3)c3cccc4c3c3ccccc3n4-c3ccccc3)cc2-c2ccccc2)cc1 ; c1ccc(-c2ccc(N(c3ccc(-c4cccc5c4sc4ccccc45)c(-c4ccccc4)c3)c3cccc4c3c3ccccc3n4-c3ccccc3)cc2-c2ccccc2)cc1]

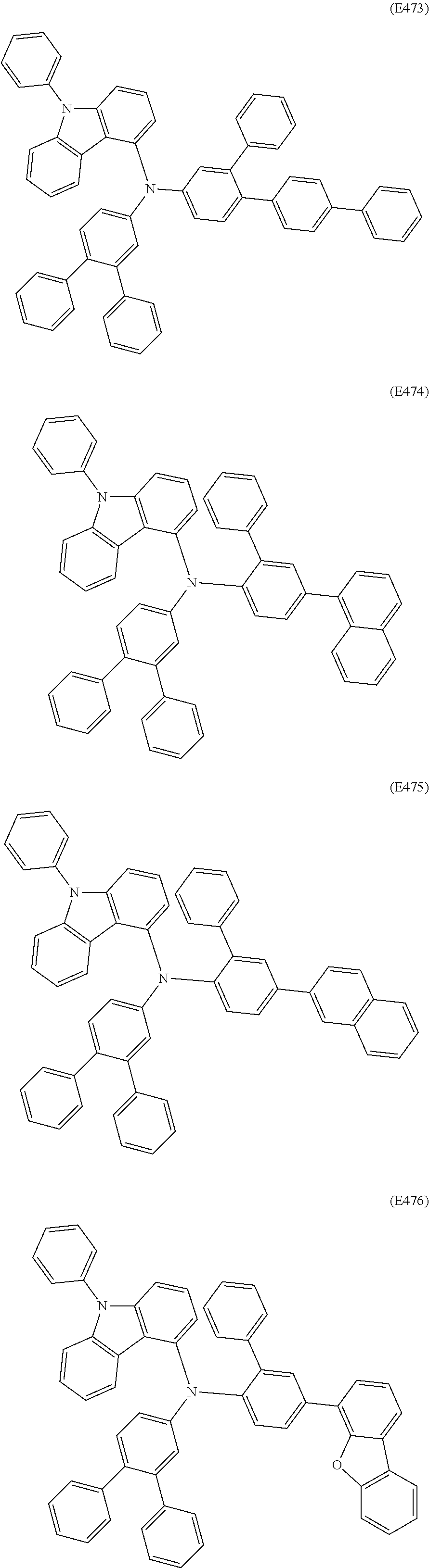 [SMILES: c1ccc(-c2ccc(-c3ccc(N(c4ccc(-c5ccccc5)c(-c5ccccc5)c4)c4cccc5c4c4ccccc4n5-c4ccccc4)cc3-c3ccccc3)cc2)cc1 ; c1ccc(-c2ccc(N(c3ccc(-c4cccc5ccccc45)cc3-c3ccccc3)c3cccc4c3c3ccccc3n4-c3ccccc3)cc2-c2ccccc2)cc1 ; c1ccc(-c2ccc(N(c3ccc(-c4ccc5ccccc5c4)cc3-c3ccccc3)c3cccc4c3c3ccccc3n4-c3ccccc3)cc2-c2ccccc2)cc1 ; c1ccc(-c2ccc(N(c3ccc(-c4cccc5c4oc4ccccc45)cc3-c3ccccc3)c3cccc4c3c3ccccc3n4-c3ccccc3)cc2-c2ccccc2)cc1]

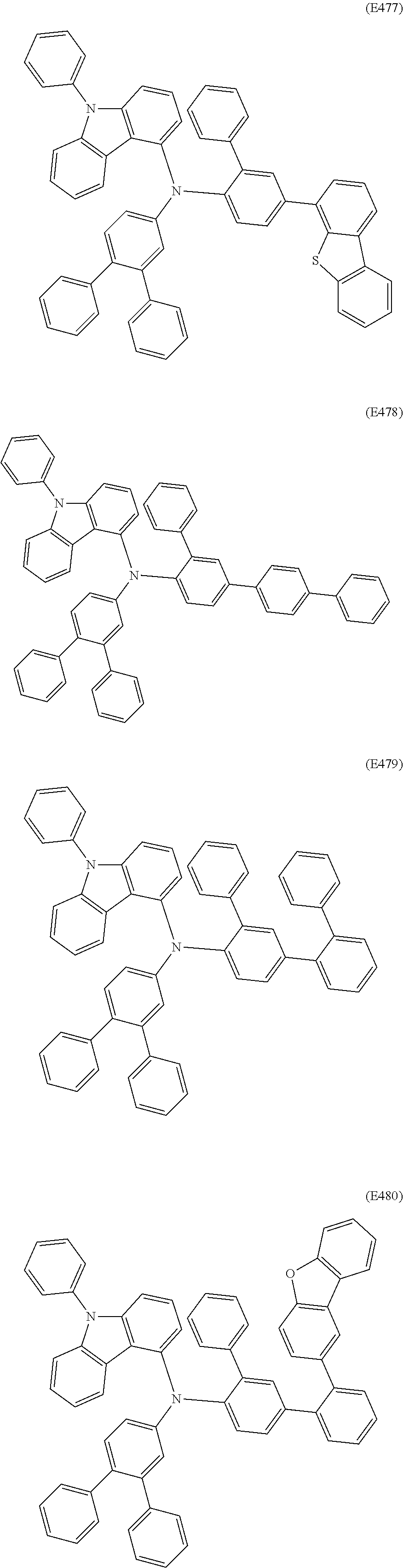 [SMILES: c1ccc(-c2ccc(N(c3ccc(-c4cccc5c4sc4ccccc45)cc3-c3ccccc3)c3cccc4c3c3ccccc3n4-c3ccccc3)cc2-c2ccccc2)cc1 ; c1ccc(-c2ccc(-c3ccc(N(c4ccc(-c5ccccc5)c(-c5ccccc5)c4)c4cccc5c4c4ccccc4n5-c4ccccc4)c(-c4ccccc4)c3)cc2)cc1 ; c1ccc(-c2ccccc2-c2ccc(N(c3ccc(-c4ccccc4)c(-c4ccccc4)c3)c3cccc4c3c3ccccc3n4-c3ccccc3)c(-c3ccccc3)c2)cc1 ; c1ccc(-c2ccc(N(c3ccc(-c4ccccc4-c4ccc5oc6ccccc6c5c4)cc3-c3ccccc3)c3cccc4c3c3ccccc3n4-c3ccccc3)cc2-c2ccccc2)cc1]

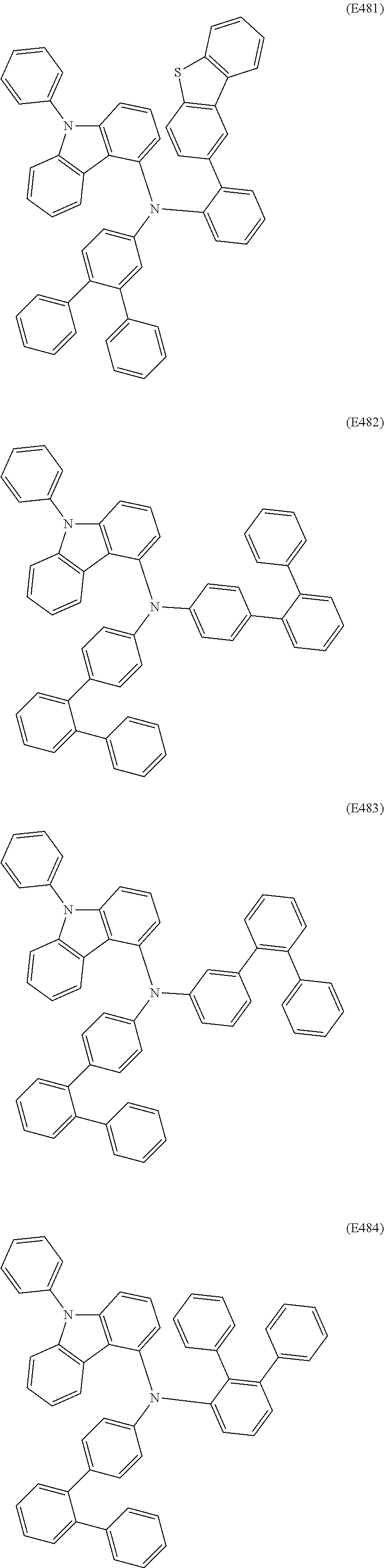 [SMILES: c1ccc(-c2ccc(N(c3ccccc3-c3ccc4sc5ccccc5c4c3)c3cccc4c3c3ccccc3n4-c3ccccc3)cc2-c2ccccc2)cc1 ; c1ccc(-c2ccccc2-c2ccc(N(c3ccc(-c4ccccc4-c4ccccc4)cc3)c3cccc4c3c3ccccc3n4-c3ccccc3)cc2)cc1 ; c1ccc(-c2ccccc2-c2ccc(N(c3cccc(-c4ccccc4-c4ccccc4)c3)c3cccc4c3c3ccccc3n4-c3ccccc3)cc2)cc1 ; c1ccc(-c2ccccc2-c2ccc(N(c3cccc(-c4ccccc4)c3-c3ccccc3)c3cccc4c3c3ccccc3n4-c3ccccc3)cc2)cc1]

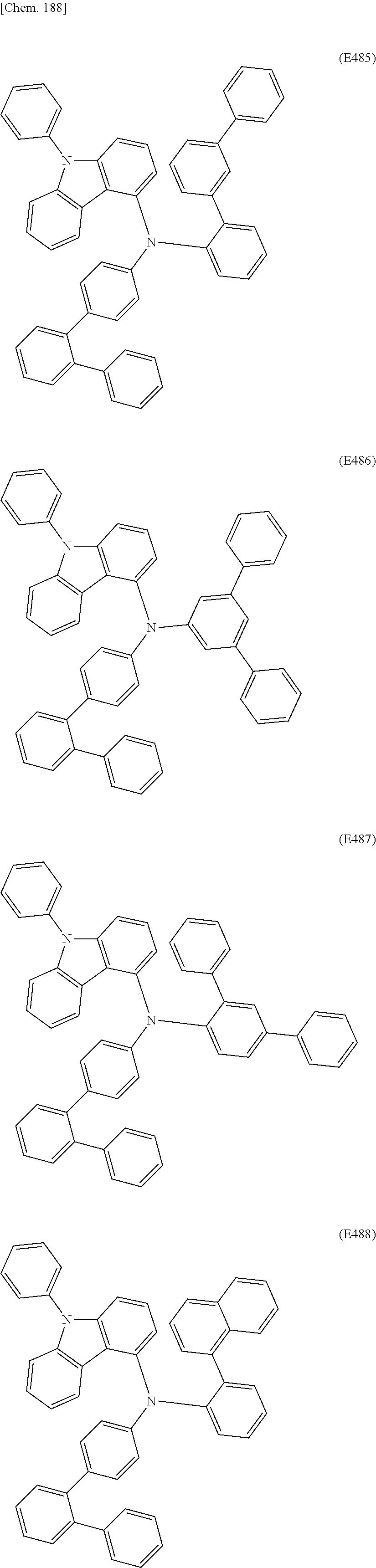 [SMILES: c1ccc(-c2cccc(-c3ccccc3N(c3ccc(-c4ccccc4-c4ccccc4)cc3)c3cccc4c3c3ccccc3n4-c3ccccc3)c2)cc1 ; c1ccc(-c2cc(-c3ccccc3)cc(N(c3ccc(-c4ccccc4-c4ccccc4)cc3)c3cccc4c3c3ccccc3n4-c3ccccc3)c2)cc1 ; c1ccc(-c2ccc(N(c3ccc(-c4ccccc4-c4ccccc4)cc3)c3cccc4c3c3ccccc3n4-c3ccccc3)c(-c3ccccc3)c2)cc1 ; c1ccc(-c2ccccc2-c2ccc(N(c3ccccc3-c3cccc4ccccc34)c3cccc4c3c3ccccc3n4-c3ccccc3)cc2)cc1]

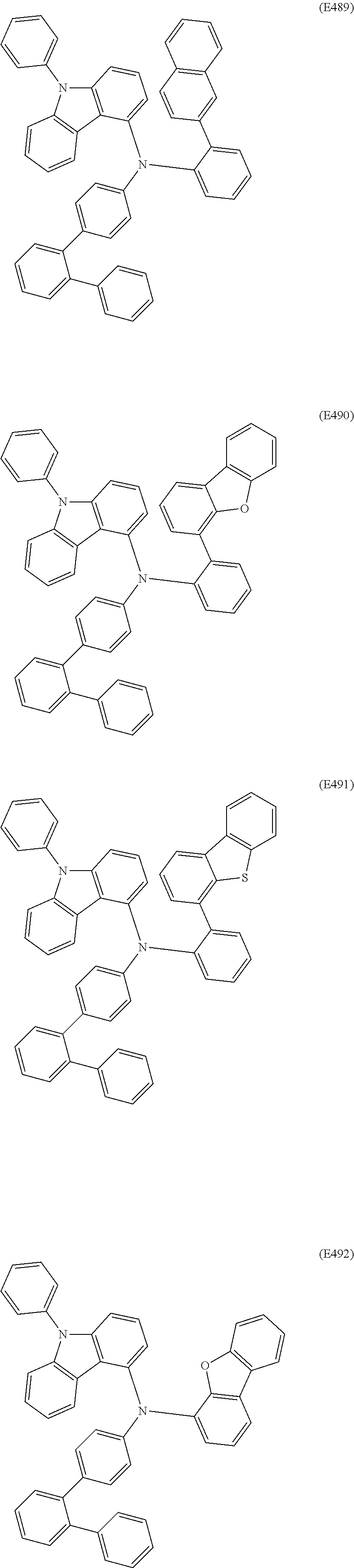 [SMILES: c1ccc(-c2ccccc2-c2ccc(N(c3ccccc3-c3ccc4ccccc4c3)c3cccc4c3c3ccccc3n4-c3ccccc3)cc2)cc1 ; c1ccc(-c2ccccc2-c2ccc(N(c3ccccc3-c3cccc4c3oc3ccccc34)c3cccc4c3c3ccccc3n4-c3ccccc3)cc2)cc1 ; c1ccc(-c2ccccc2-c2ccc(N(c3ccccc3-c3cccc4c3sc3ccccc34)c3cccc4c3c3ccccc3n4-c3ccccc3)cc2)cc1 ; c1ccc(-c2ccccc2-c2ccc(N(c3cccc4c3oc3ccccc34)c3cccc4c3c3ccccc3n4-c3ccccc3)cc2)cc1]

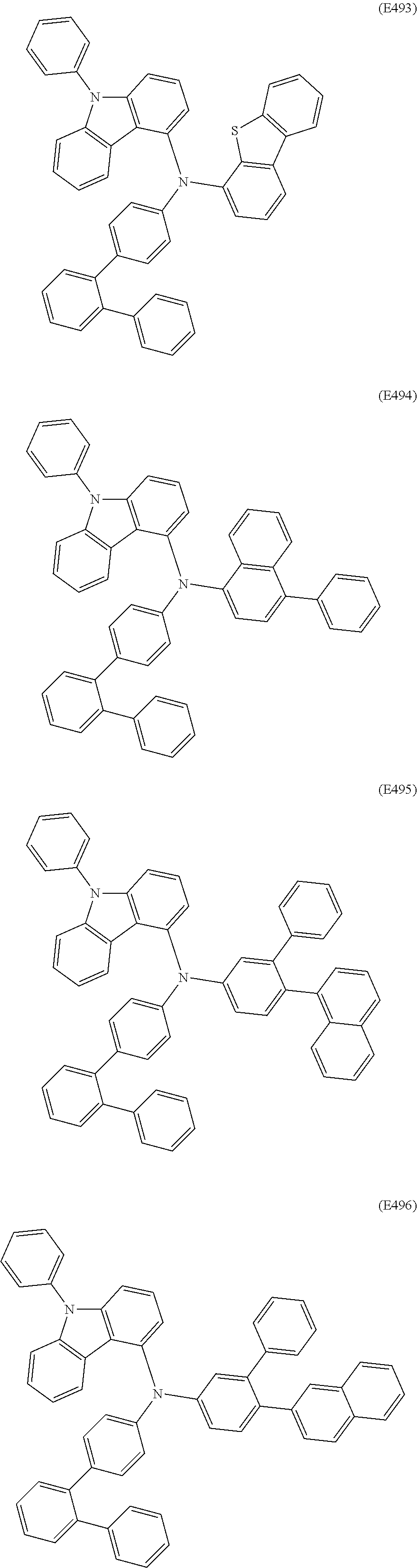 [SMILES: c1ccc(-c2ccccc2-c2ccc(N(c3cccc4c3sc3ccccc34)c3cccc4c3c3ccccc3n4-c3ccccc3)cc2)cc1 ; c1ccc(-c2ccccc2-c2ccc(N(c3ccc(-c4ccccc4)c4ccccc34)c3cccc4c3c3ccccc3n4-c3ccccc3)cc2)cc1 ; c1ccc(-c2ccccc2-c2ccc(N(c3ccc(-c4cccc5ccccc45)c(-c4ccccc4)c3)c3cccc4c3c3ccccc3n4-c3ccccc3)cc2)cc1 ; c1ccc(-c2ccccc2-c2ccc(N(c3ccc(-c4ccc5ccccc5c4)c(-c4ccccc4)c3)c3cccc4c3c3ccccc3n4-c3ccccc3)cc2)cc1]

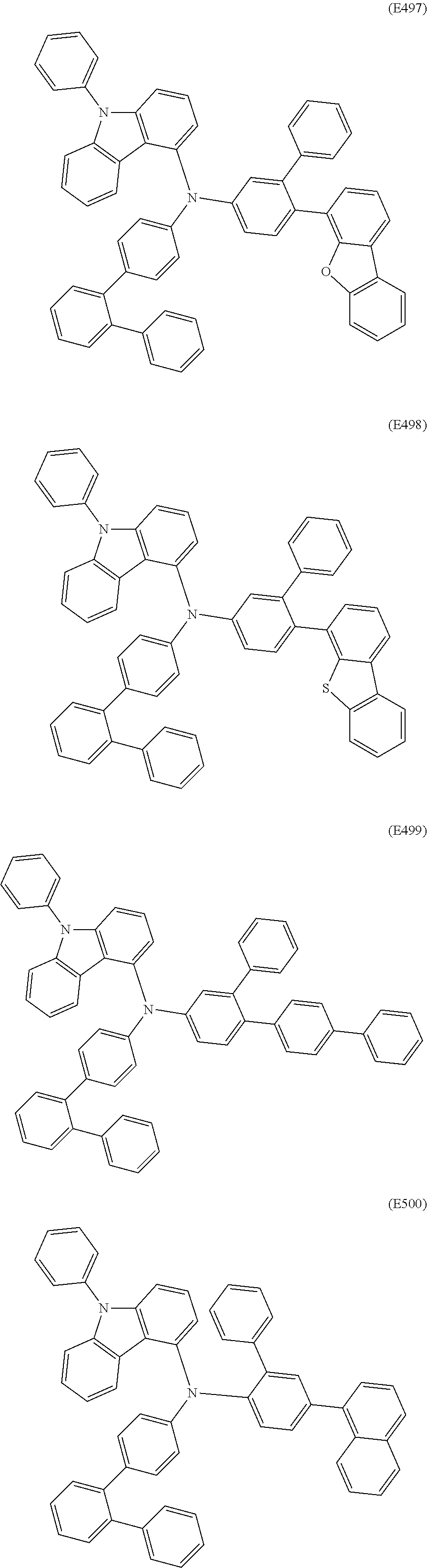 [SMILES: c1ccc(-c2ccccc2-c2ccc(N(c3ccc(-c4cccc5c4oc4ccccc45)c(-c4ccccc4)c3)c3cccc4c3c3ccccc3n4-c3ccccc3)cc2)cc1 ; c1ccc(-c2ccccc2-c2ccc(N(c3ccc(-c4cccc5c4sc4ccccc45)c(-c4ccccc4)c3)c3cccc4c3c3ccccc3n4-c3ccccc3)cc2)cc1 ; c1ccc(-c2ccc(-c3ccc(N(c4ccc(-c5ccccc5-c5ccccc5)cc4)c4cccc5c4c4ccccc4n5-c4ccccc4)cc3-c3ccccc3)cc2)cc1 ; c1ccc(-c2ccccc2-c2ccc(N(c3ccc(-c4cccc5ccccc45)cc3-c3ccccc3)c3cccc4c3c3ccccc3n4-c3ccccc3)cc2)cc1]

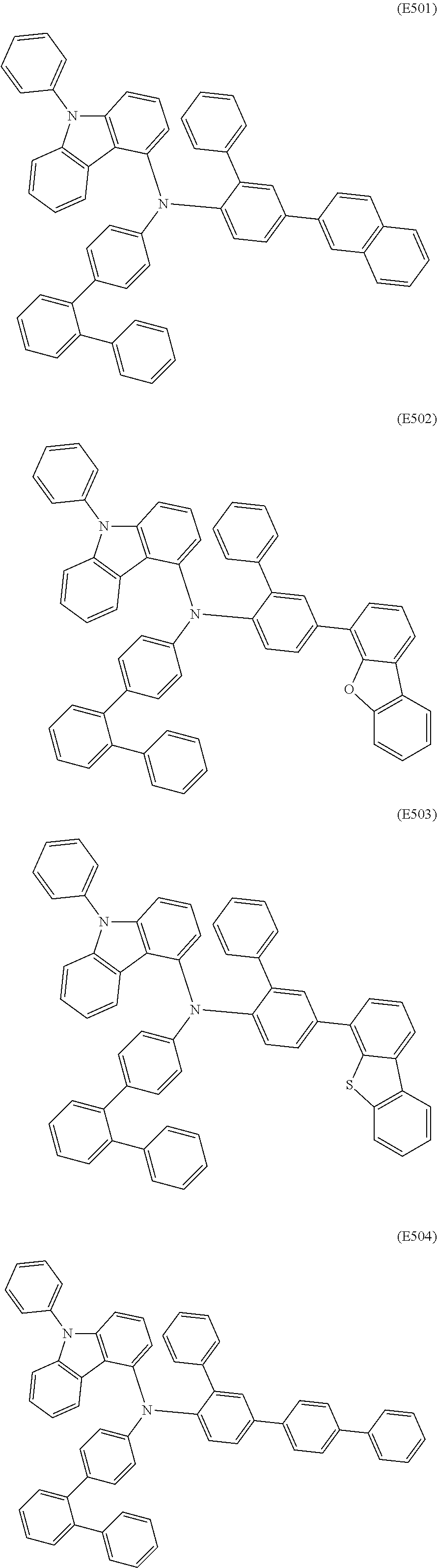 [SMILES: c1ccc(-c2ccccc2-c2ccc(N(c3ccc(-c4ccc5ccccc5c4)cc3-c3ccccc3)c3cccc4c3c3ccccc3n4-c3ccccc3)cc2)cc1 ; c1ccc(-c2ccccc2-c2ccc(N(c3ccc(-c4cccc5c4oc4ccccc45)cc3-c3ccccc3)c3cccc4c3c3ccccc3n4-c3ccccc3)cc2)cc1 ; c1ccc(-c2ccccc2-c2ccc(N(c3ccc(-c4cccc5c4sc4ccccc45)cc3-c3ccccc3)c3cccc4c3c3ccccc3n4-c3ccccc3)cc2)cc1 ; c1ccc(-c2ccc(-c3ccc(N(c4ccc(-c5ccccc5-c5ccccc5)cc4)c4cccc5c4c4ccccc4n5-c4ccccc4)c(-c4ccccc4)c3)cc2)cc1]

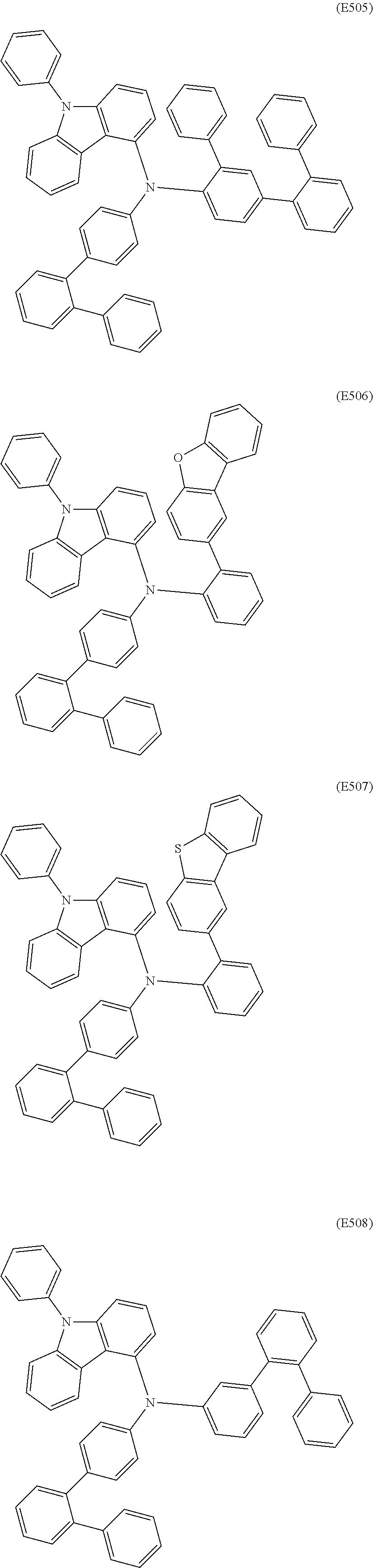 [SMILES: c1ccc(-c2ccccc2-c2ccc(N(c3ccc(-c4ccccc4-c4ccccc4)cc3-c3ccccc3)c3cccc4c3c3ccccc3n4-c3ccccc3)cc2)cc1 ; c1ccc(-c2ccccc2-c2ccc(N(c3ccccc3-c3ccc4oc5ccccc5c4c3)c3cccc4c3c3ccccc3n4-c3ccccc3)cc2)cc1 ; c1ccc(-c2ccccc2-c2ccc(N(c3ccccc3-c3ccc4sc5ccccc5c4c3)c3cccc4c3c3ccccc3n4-c3ccccc3)cc2)cc1 ; c1ccc(-c2ccccc2-c2ccc(N(c3cccc(-c4ccccc4-c4ccccc4)c3)c3cccc4c3c3ccccc3n4-c3ccccc3)cc2)cc1]

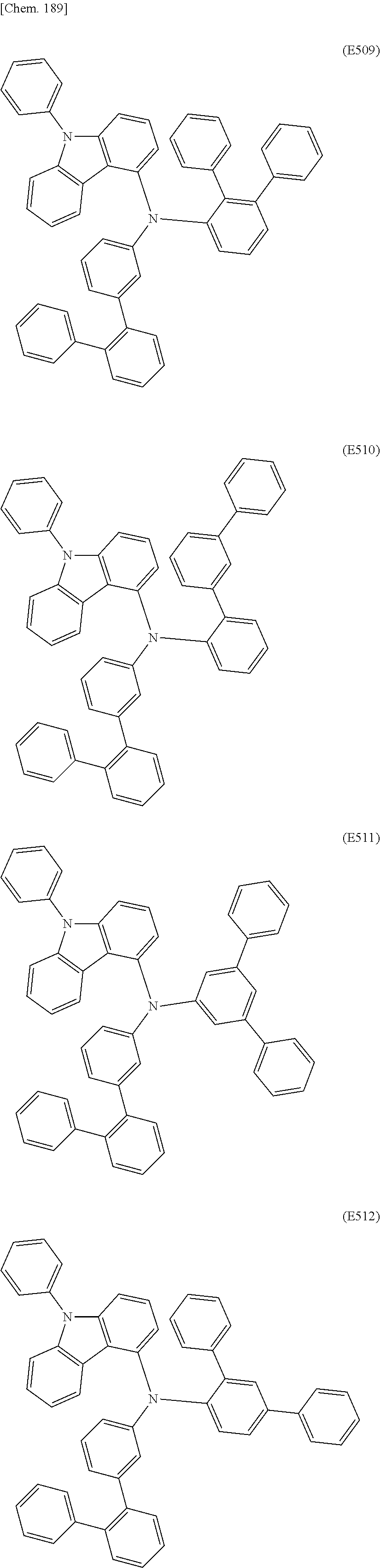 [SMILES: c1ccc(-c2ccccc2-c2cccc(N(c3cccc(-c4ccccc4)c3-c3ccccc3)c3cccc4c3c3ccccc3n4-c3ccccc3)c2)cc1 ; c1ccc(-c2cccc(-c3ccccc3N(c3cccc(-c4ccccc4-c4ccccc4)c3)c3cccc4c3c3ccccc3n4-c3ccccc3)c2)cc1 ; c1ccc(-c2cc(-c3ccccc3)cc(N(c3cccc(-c4ccccc4-c4ccccc4)c3)c3cccc4c3c3ccccc3n4-c3ccccc3)c2)cc1 ; c1ccc(-c2ccc(N(c3cccc(-c4ccccc4-c4ccccc4)c3)c3cccc4c3c3ccccc3n4-c3ccccc3)c(-c3ccccc3)c2)cc1]

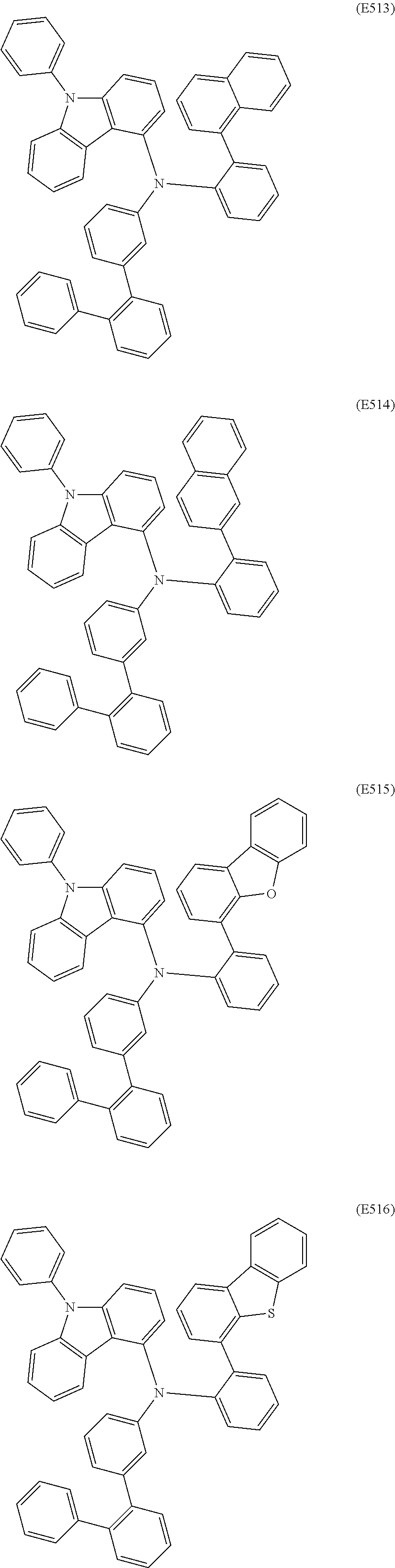 [SMILES: c1ccc(-c2ccccc2-c2cccc(N(c3ccccc3-c3cccc4ccccc34)c3cccc4c3c3ccccc3n4-c3ccccc3)c2)cc1 ; c1ccc(-c2ccccc2-c2cccc(N(c3ccccc3-c3ccc4ccccc4c3)c3cccc4c3c3ccccc3n4-c3ccccc3)c2)cc1 ; c1ccc(-c2ccccc2-c2cccc(N(c3ccccc3-c3cccc4c3oc3ccccc34)c3cccc4c3c3ccccc3n4-c3ccccc3)c2)cc1 ; c1ccc(-c2ccccc2-c2cccc(N(c3ccccc3-c3cccc4c3sc3ccccc34)c3cccc4c3c3ccccc3n4-c3ccccc3)c2)cc1]

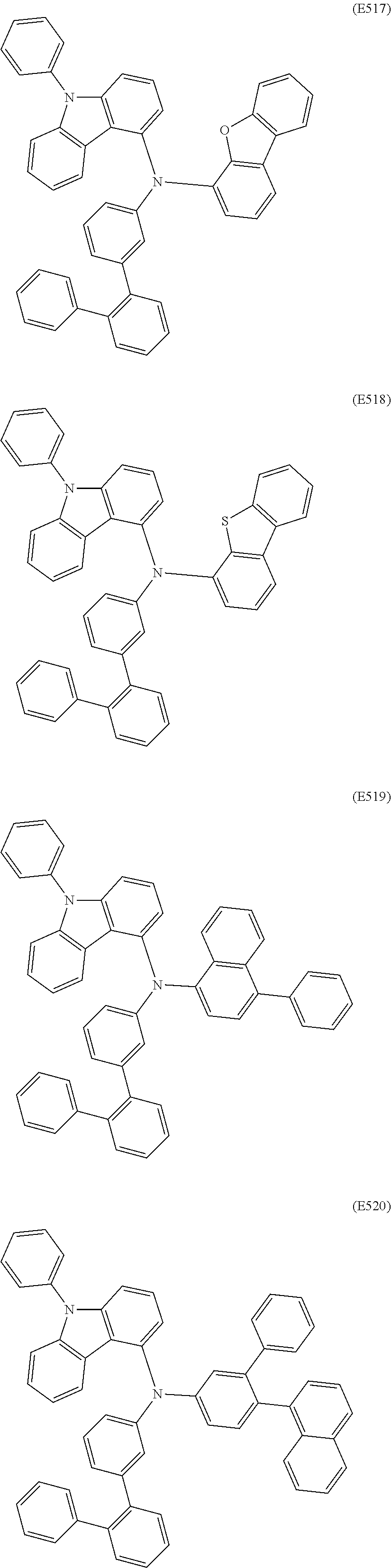 [SMILES: c1ccc(-c2ccccc2-c2cccc(N(c3cccc4c3oc3ccccc34)c3cccc4c3c3ccccc3n4-c3ccccc3)c2)cc1 ; c1ccc(-c2ccccc2-c2cccc(N(c3cccc4c3sc3ccccc34)c3cccc4c3c3ccccc3n4-c3ccccc3)c2)cc1 ; c1ccc(-c2ccccc2-c2cccc(N(c3ccc(-c4ccccc4)c4ccccc34)c3cccc4c3c3ccccc3n4-c3ccccc3)c2)cc1 ; c1ccc(-c2ccccc2-c2cccc(N(c3ccc(-c4cccc5ccccc45)c(-c4ccccc4)c3)c3cccc4c3c3ccccc3n4-c3ccccc3)c2)cc1]

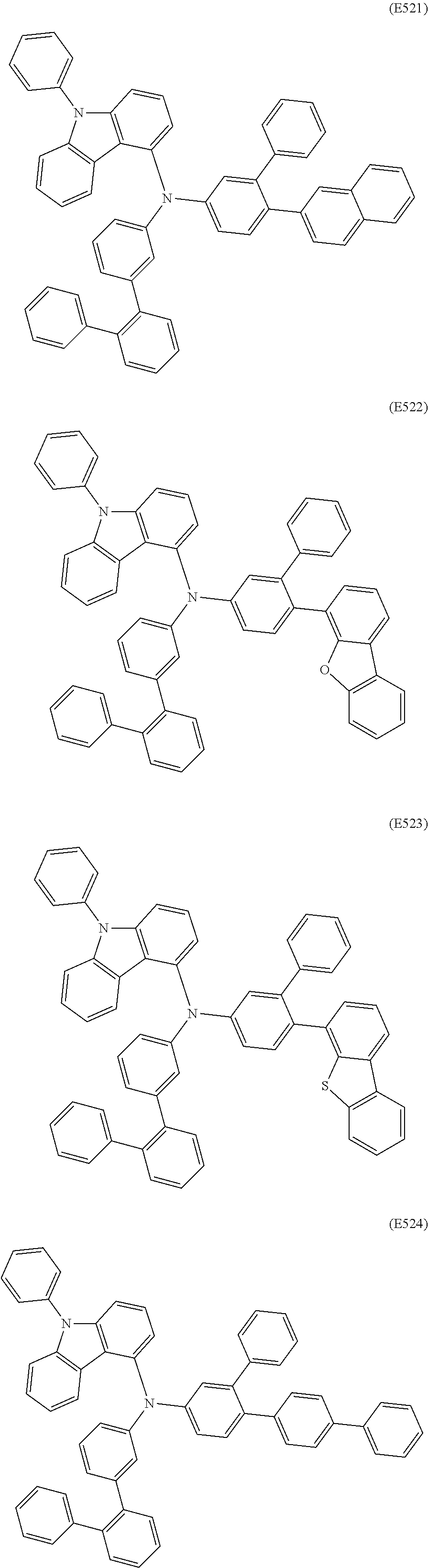 [SMILES: c1ccc(-c2ccccc2-c2cccc(N(c3ccc(-c4ccc5ccccc5c4)c(-c4ccccc4)c3)c3cccc4c3c3ccccc3n4-c3ccccc3)c2)cc1 ; c1ccc(-c2ccccc2-c2cccc(N(c3ccc(-c4cccc5c4oc4ccccc45)c(-c4ccccc4)c3)c3cccc4c3c3ccccc3n4-c3ccccc3)c2)cc1 ; c1ccc(-c2ccccc2-c2cccc(N(c3ccc(-c4cccc5c4sc4ccccc45)c(-c4ccccc4)c3)c3cccc4c3c3ccccc3n4-c3ccccc3)c2)cc1 ; c1ccc(-c2ccc(-c3ccc(N(c4cccc(-c5ccccc5-c5ccccc5)c4)c4cccc5c4c4ccccc4n5-c4ccccc4)cc3-c3ccccc3)cc2)cc1]

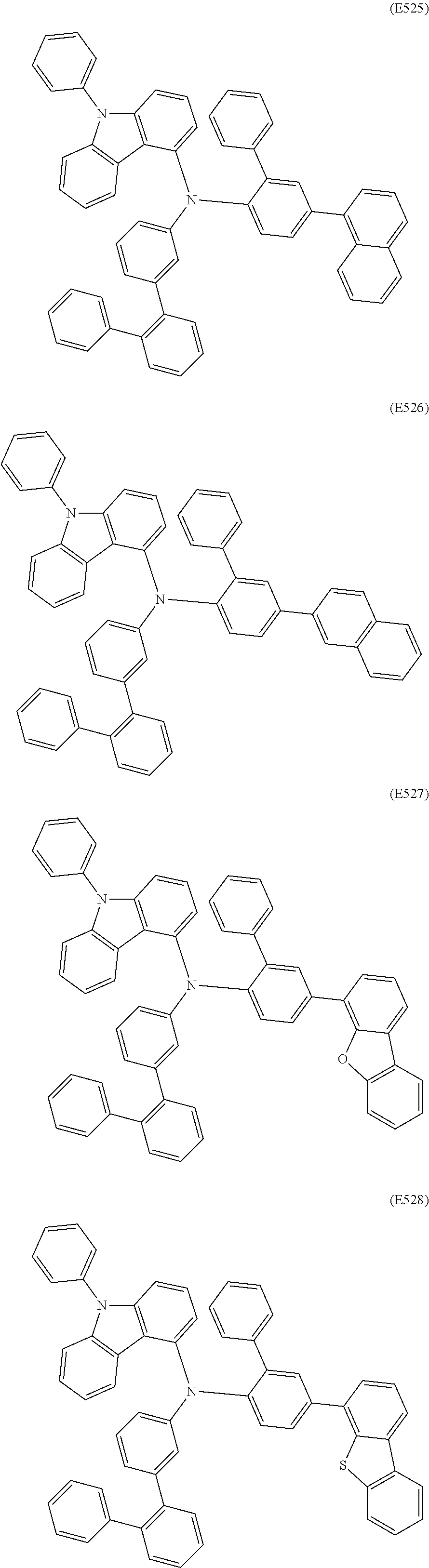 [SMILES: c1ccc(-c2ccccc2-c2cccc(N(c3ccc(-c4cccc5ccccc45)cc3-c3ccccc3)c3cccc4c3c3ccccc3n4-c3ccccc3)c2)cc1 ; c1ccc(-c2ccccc2-c2cccc(N(c3ccc(-c4ccc5ccccc5c4)cc3-c3ccccc3)c3cccc4c3c3ccccc3n4-c3ccccc3)c2)cc1 ; c1ccc(-c2ccccc2-c2cccc(N(c3ccc(-c4cccc5c4oc4ccccc45)cc3-c3ccccc3)c3cccc4c3c3ccccc3n4-c3ccccc3)c2)cc1 ; c1ccc(-c2ccccc2-c2cccc(N(c3ccc(-c4cccc5c4sc4ccccc45)cc3-c3ccccc3)c3cccc4c3c3ccccc3n4-c3ccccc3)c2)cc1]

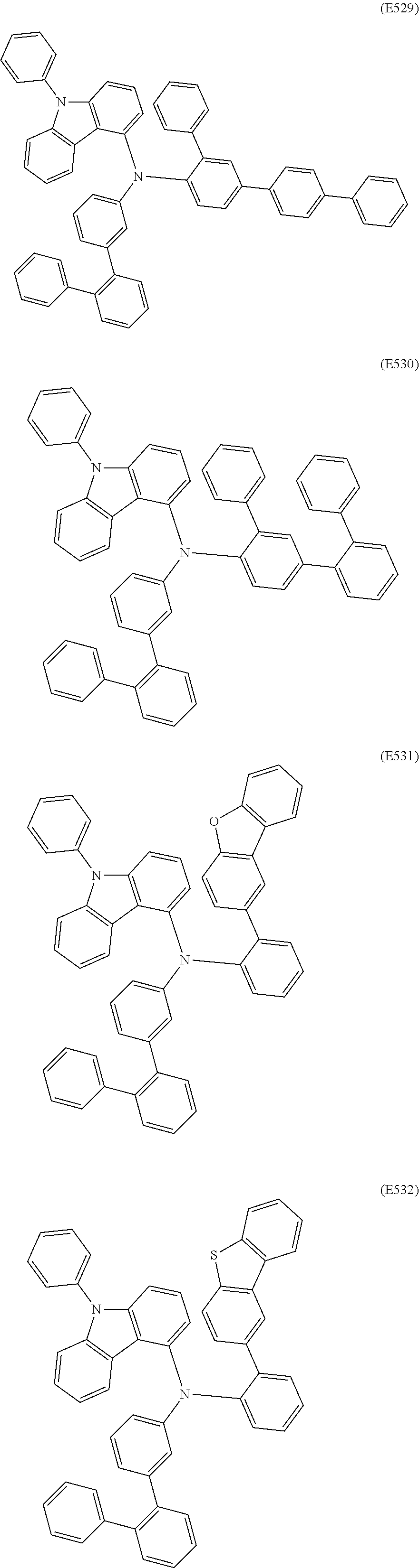 [SMILES: c1ccc(-c2ccc(-c3ccc(N(c4cccc(-c5ccccc5-c5ccccc5)c4)c4cccc5c4c4ccccc4n5-c4ccccc4)c(-c4ccccc4)c3)cc2)cc1 ; c1ccc(-c2ccccc2-c2cccc(N(c3ccc(-c4ccccc4-c4ccccc4)cc3-c3ccccc3)c3cccc4c3c3ccccc3n4-c3ccccc3)c2)cc1 ; c1ccc(-c2ccccc2-c2cccc(N(c3ccccc3-c3ccc4oc5ccccc5c4c3)c3cccc4c3c3ccccc3n4-c3ccccc3)c2)cc1 ; c1ccc(-c2ccccc2-c2cccc(N(c3ccccc3-c3ccc4sc5ccccc5c4c3)c3cccc4c3c3ccccc3n4-c3ccccc3)c2)cc1]

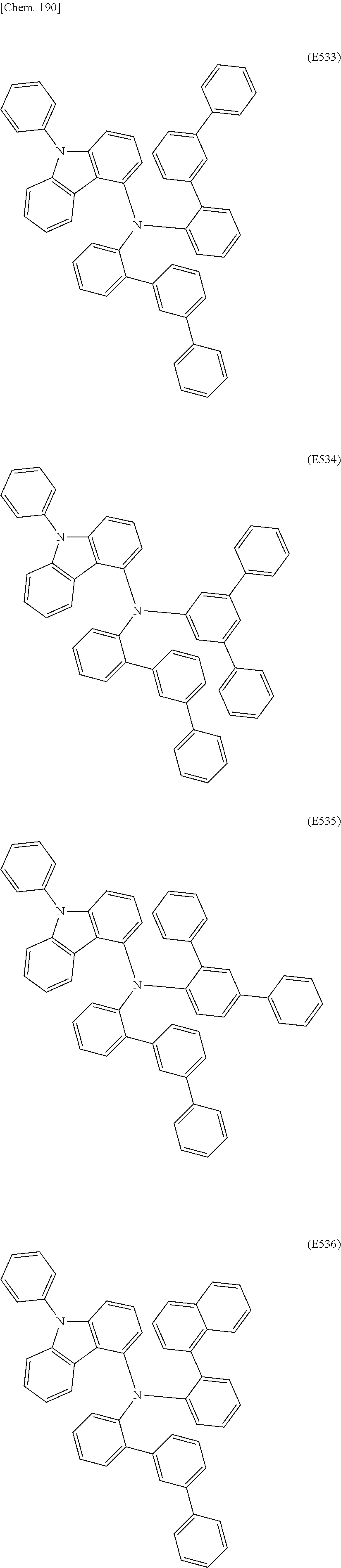 [SMILES: c1ccc(-c2cccc(-c3ccccc3N(c3ccccc3-c3cccc(-c4ccccc4)c3)c3cccc4c3c3ccccc3n4-c3ccccc3)c2)cc1 ; c1ccc(-c2cccc(-c3ccccc3N(c3cc(-c4ccccc4)cc(-c4ccccc4)c3)c3cccc4c3c3ccccc3n4-c3ccccc3)c2)cc1 ; c1ccc(-c2cccc(-c3ccccc3N(c3ccc(-c4ccccc4)cc3-c3ccccc3)c3cccc4c3c3ccccc3n4-c3ccccc3)c2)cc1 ; c1ccc(-c2cccc(-c3ccccc3N(c3ccccc3-c3cccc4ccccc34)c3cccc4c3c3ccccc3n4-c3ccccc3)c2)cc1]

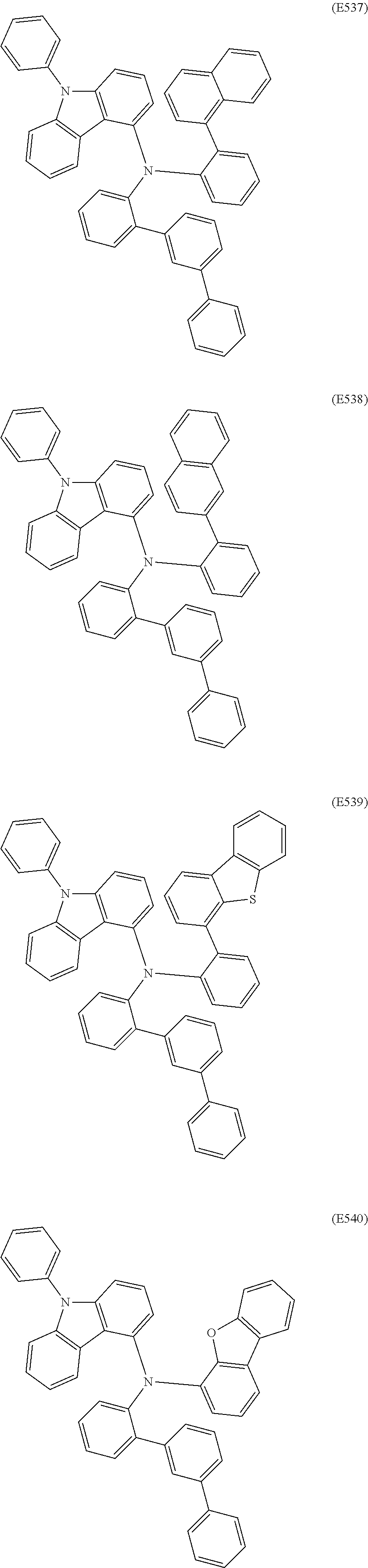 [SMILES: c1ccc(-c2cccc(-c3ccccc3N(c3ccccc3-c3cccc4ccccc34)c3cccc4c3c3ccccc3n4-c3ccccc3)c2)cc1 ; c1ccc(-c2cccc(-c3ccccc3N(c3ccccc3-c3ccc4ccccc4c3)c3cccc4c3c3ccccc3n4-c3ccccc3)c2)cc1 ; c1ccc(-c2cccc(-c3ccccc3N(c3ccccc3-c3cccc4c3sc3ccccc34)c3cccc4c3c3ccccc3n4-c3ccccc3)c2)cc1 ; c1ccc(-c2cccc(-c3ccccc3N(c3cccc4c3oc3ccccc34)c3cccc4c3c3ccccc3n4-c3ccccc3)c2)cc1]

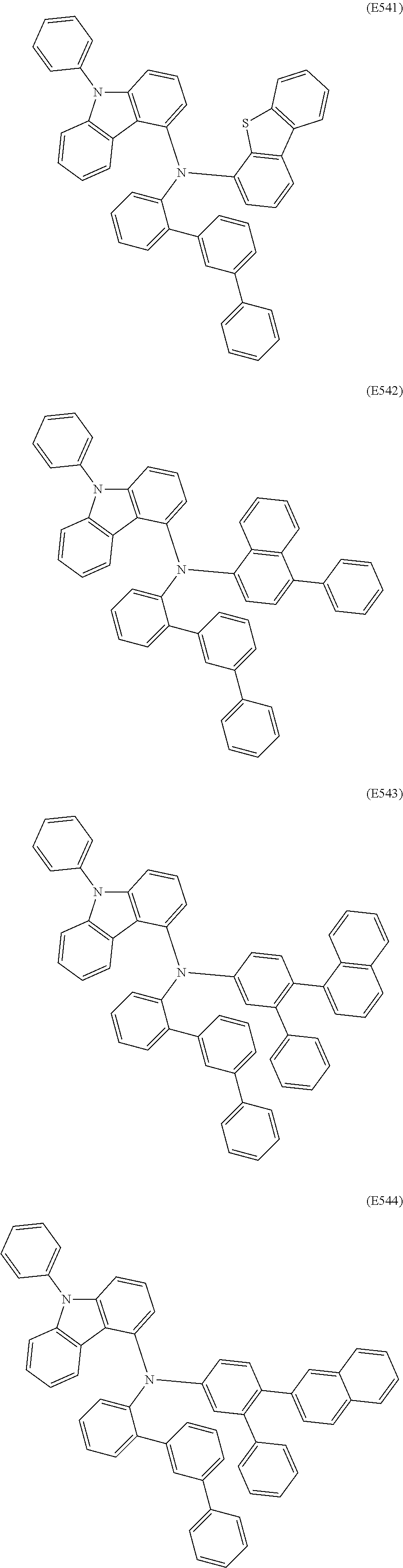 [SMILES: c1ccc(-c2cccc(-c3ccccc3N(c3cccc4c3sc3ccccc34)c3cccc4c3c3ccccc3n4-c3ccccc3)c2)cc1 ; c1ccc(-c2cccc(-c3ccccc3N(c3ccc(-c4ccccc4)c4ccccc34)c3cccc4c3c3ccccc3n4-c3ccccc3)c2)cc1 ; c1ccc(-c2cccc(-c3ccccc3N(c3ccc(-c4cccc5ccccc45)c(-c4ccccc4)c3)c3cccc4c3c3ccccc3n4-c3ccccc3)c2)cc1 ; c1ccc(-c2cccc(-c3ccccc3N(c3ccc(-c4ccc5ccccc5c4)c(-c4ccccc4)c3)c3cccc4c3c3ccccc3n4-c3ccccc3)c2)cc1]

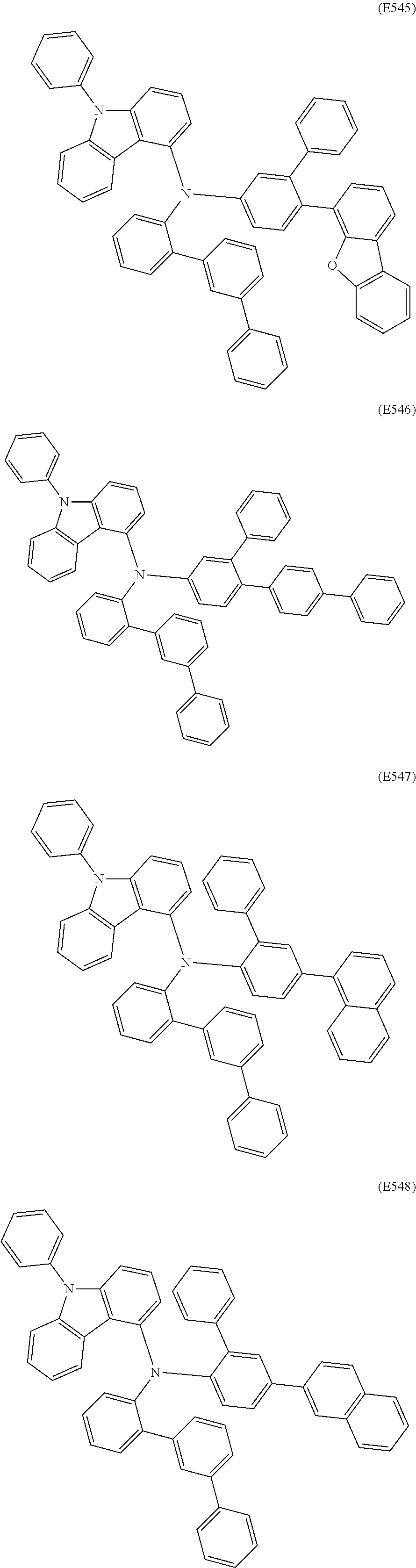 [SMILES: c1ccc(-c2cccc(-c3ccccc3N(c3ccc(-c4cccc5c4oc4ccccc45)c(-c4ccccc4)c3)c3cccc4c3c3ccccc3n4-c3ccccc3)c2)cc1 ; c1ccc(-c2ccc(-c3ccc(N(c4ccccc4-c4cccc(-c5ccccc5)c4)c4cccc5c4c4ccccc4n5-c4ccccc4)cc3-c3ccccc3)cc2)cc1 ; c1ccc(-c2cccc(-c3ccccc3N(c3ccc(-c4cccc5ccccc45)cc3-c3ccccc3)c3cccc4c3c3ccccc3n4-c3ccccc3)c2)cc1 ; c1ccc(-c2cccc(-c3ccccc3N(c3ccc(-c4ccc5ccccc5c4)cc3-c3ccccc3)c3cccc4c3c3ccccc3n4-c3ccccc3)c2)cc1]

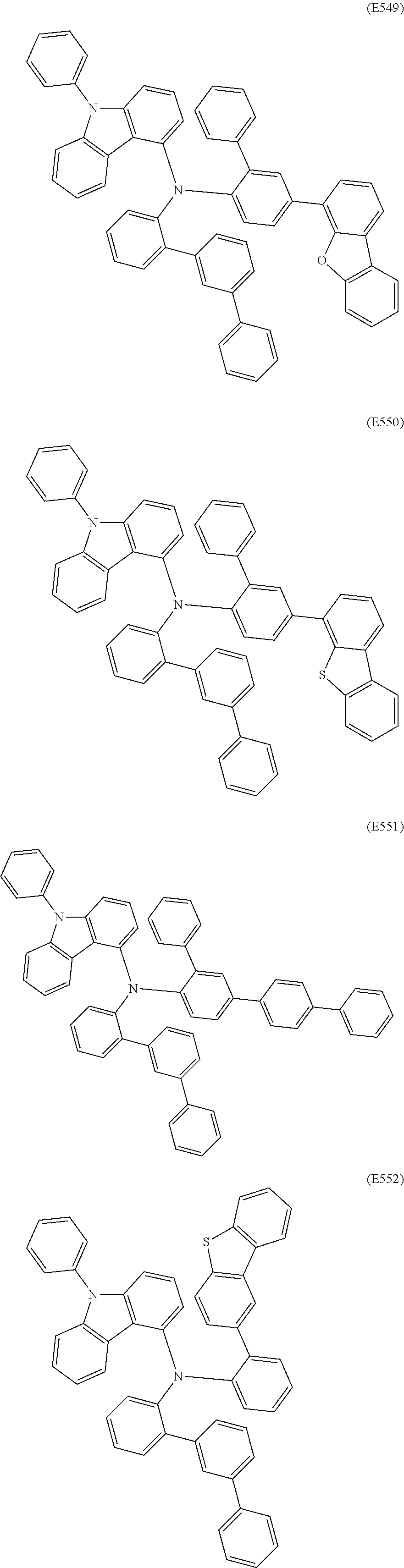 [SMILES: c1ccc(-c2cccc(-c3ccccc3N(c3ccc(-c4cccc5c4oc4ccccc45)cc3-c3ccccc3)c3cccc4c3c3ccccc3n4-c3ccccc3)c2)cc1 ; c1ccc(-c2cccc(-c3ccccc3N(c3ccc(-c4cccc5c4sc4ccccc45)cc3-c3ccccc3)c3cccc4c3c3ccccc3n4-c3ccccc3)c2)cc1 ; c1ccc(-c2ccc(-c3ccc(N(c4ccccc4-c4cccc(-c5ccccc5)c4)c4cccc5c4c4ccccc4n5-c4ccccc4)c(-c4ccccc4)c3)cc2)cc1 ; c1ccc(-c2cccc(-c3ccccc3N(c3ccccc3-c3ccc4sc5ccccc5c4c3)c3cccc4c3c3ccccc3n4-c3ccccc3)c2)cc1]

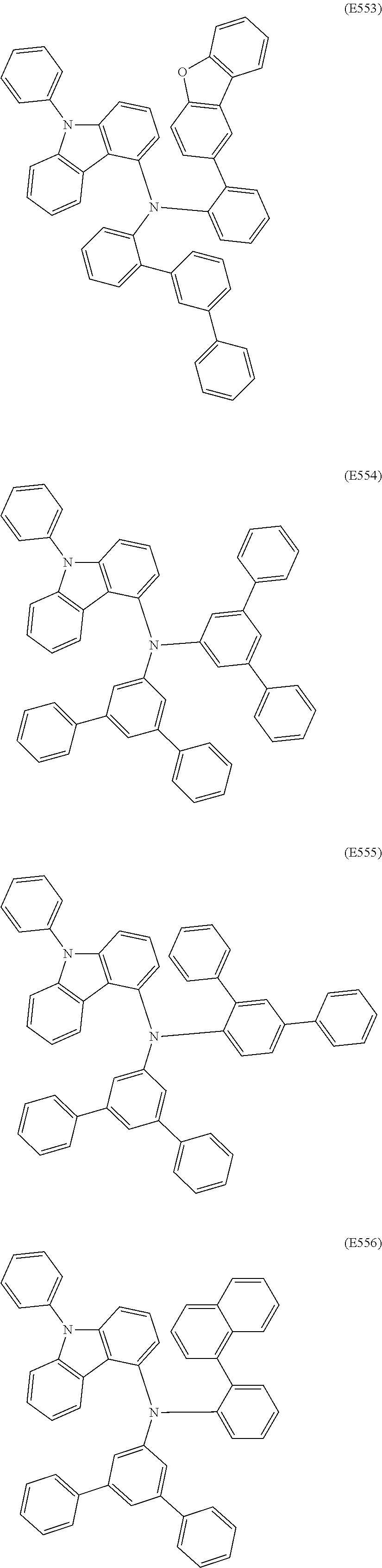 [SMILES: c1ccc(-c2cccc(-c3ccccc3N(c3ccccc3-c3ccc4oc5ccccc5c4c3)c3cccc4c3c3ccccc3n4-c3ccccc3)c2)cc1 ; c1ccc(-c2cc(-c3ccccc3)cc(N(c3cc(-c4ccccc4)cc(-c4ccccc4)c3)c3cccc4c3c3ccccc3n4-c3ccccc3)c2)cc1 ; c1ccc(-c2cc(-c3ccccc3)cc(N(c3ccc(-c4ccccc4)cc3-c3ccccc3)c3cccc4c3c3ccccc3n4-c3ccccc3)c2)cc1 ; c1ccc(-c2cc(-c3ccccc3)cc(N(c3ccccc3-c3cccc4ccccc34)c3cccc4c3c3ccccc3n4-c3ccccc3)c2)cc1]

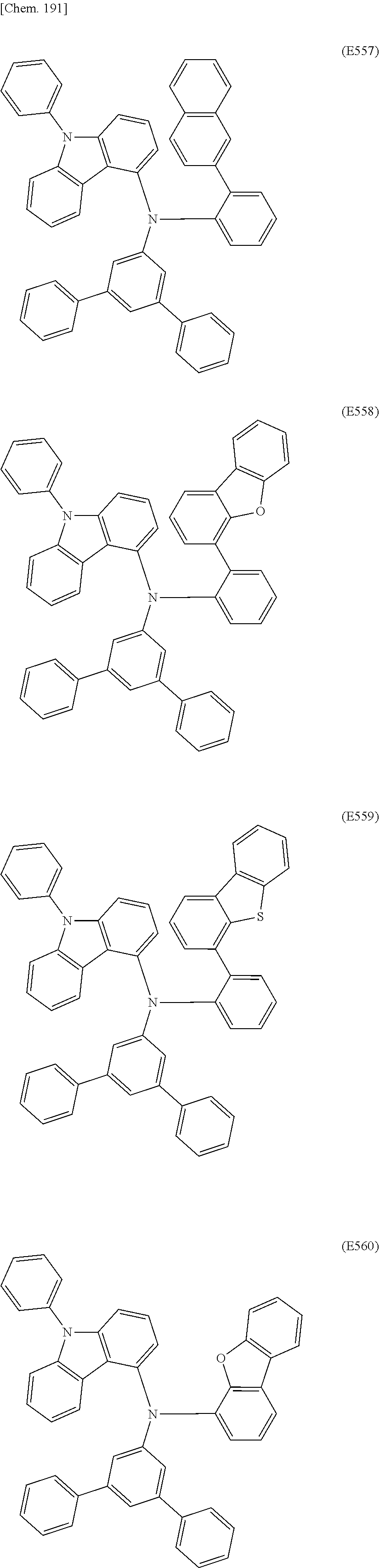 [SMILES: c1ccc(-c2cc(-c3ccccc3)cc(N(c3ccccc3-c3ccc4ccccc4c3)c3cccc4c3c3ccccc3n4-c3ccccc3)c2)cc1 ; c1ccc(-c2cc(-c3ccccc3)cc(N(c3ccccc3-c3cccc4c3oc3ccccc34)c3cccc4c3c3ccccc3n4-c3ccccc3)c2)cc1 ; c1ccc(-c2cc(-c3ccccc3)cc(N(c3ccccc3-c3cccc4c3sc3ccccc34)c3cccc4c3c3ccccc3n4-c3ccccc3)c2)cc1 ; c1ccc(-c2cc(-c3ccccc3)cc(N(c3cccc4c3oc3ccccc34)c3cccc4c3c3ccccc3n4-c3ccccc3)c2)cc1]

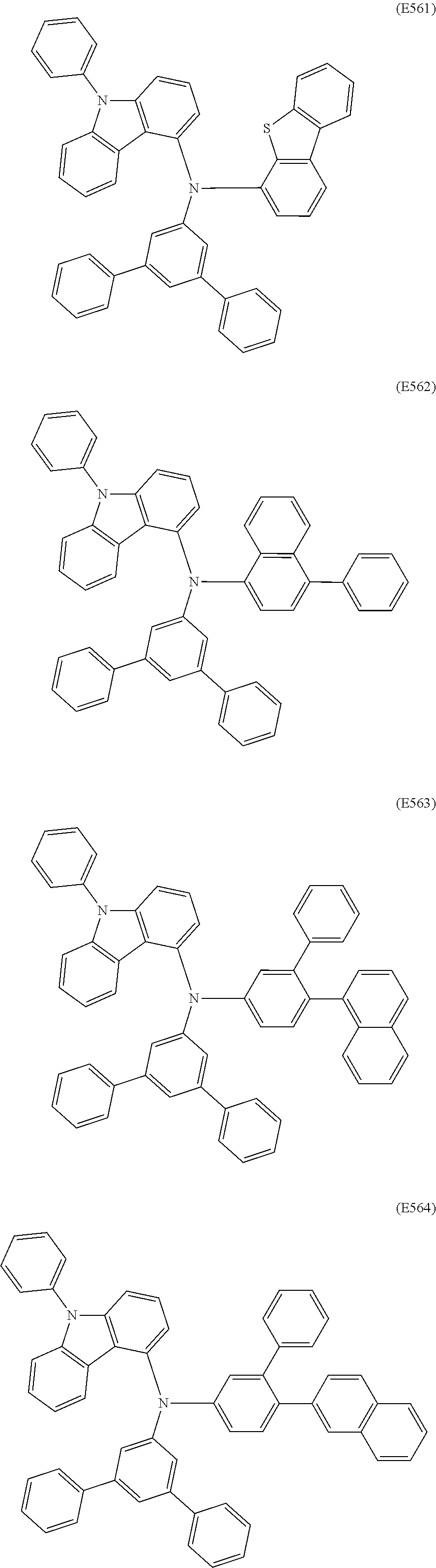 [SMILES: c1ccc(-c2cc(-c3ccccc3)cc(N(c3cccc4c3sc3ccccc34)c3cccc4c3c3ccccc3n4-c3ccccc3)c2)cc1 ; c1ccc(-c2cc(-c3ccccc3)cc(N(c3ccc(-c4ccccc4)c4ccccc34)c3cccc4c3c3ccccc3n4-c3ccccc3)c2)cc1 ; c1ccc(-c2cc(-c3ccccc3)cc(N(c3ccc(-c4cccc5ccccc45)c(-c4ccccc4)c3)c3cccc4c3c3ccccc3n4-c3ccccc3)c2)cc1 ; c1ccc(-c2cc(-c3ccccc3)cc(N(c3ccc(-c4ccc5ccccc5c4)c(-c4ccccc4)c3)c3cccc4c3c3ccccc3n4-c3ccccc3)c2)cc1]

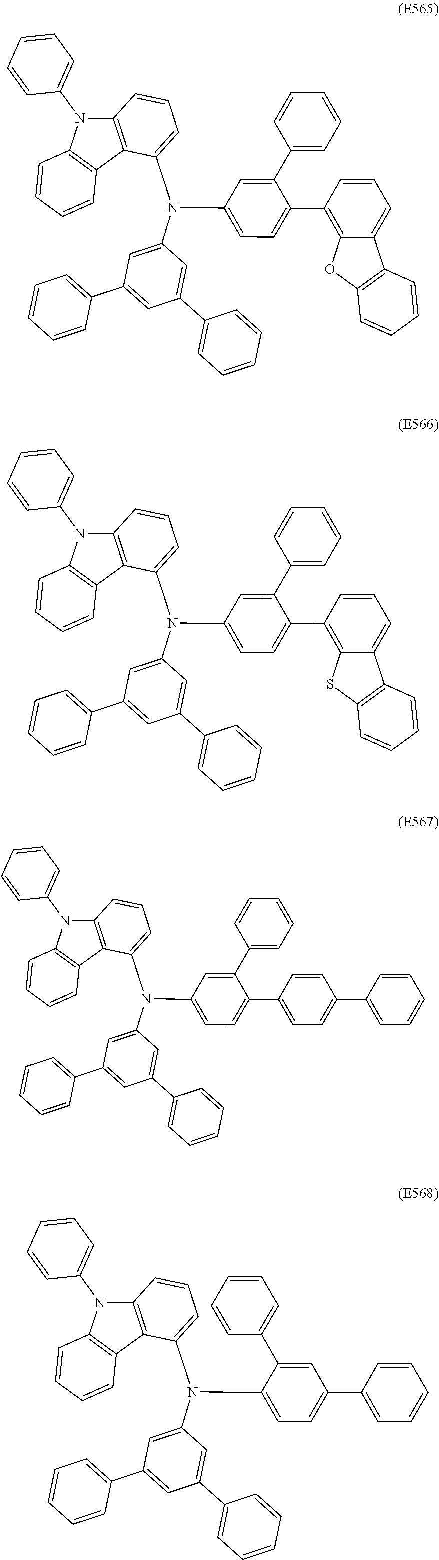 [SMILES: c1ccc(-c2cc(-c3ccccc3)cc(N(c3ccc(-c4cccc5c4oc4ccccc45)c(-c4ccccc4)c3)c3cccc4c3c3ccccc3n4-c3ccccc3)c2)cc1 ; c1ccc(-c2cc(-c3ccccc3)cc(N(c3ccc(-c4cccc5c4sc4ccccc45)c(-c4ccccc4)c3)c3cccc4c3c3ccccc3n4-c3ccccc3)c2)cc1 ; c1ccc(-c2ccc(-c3ccc(N(c4cc(-c5ccccc5)cc(-c5ccccc5)c4)c4cccc5c4c4ccccc4n5-c4ccccc4)cc3-c3ccccc3)cc2)cc1 ; c1ccc(-c2cc(-c3ccccc3)cc(N(c3ccc(-c4ccccc4)cc3-c3ccccc3)c3cccc4c3c3ccccc3n4-c3ccccc3)c2)cc1]

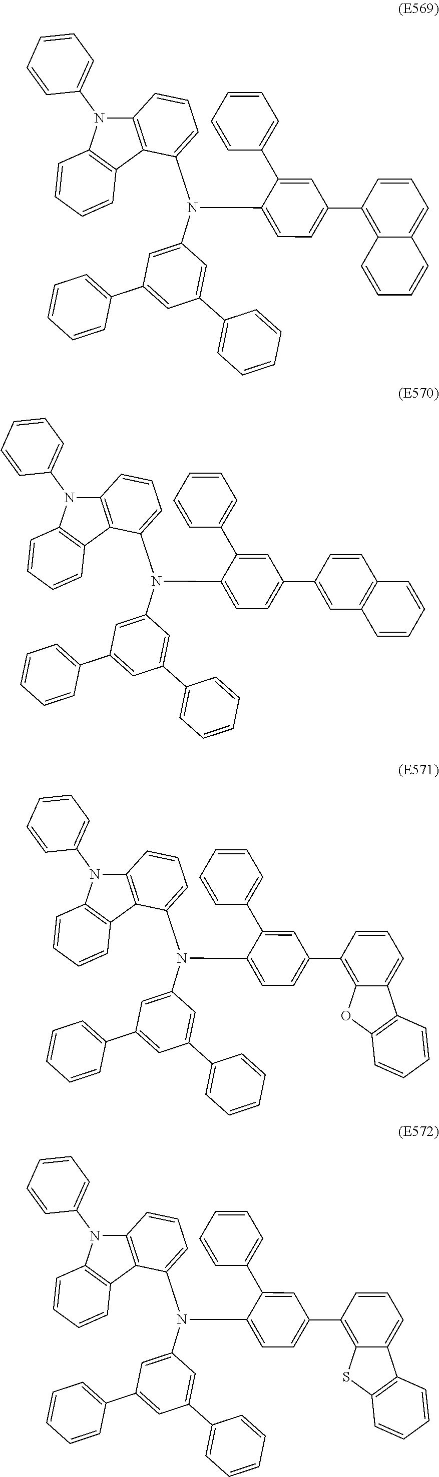 [SMILES: c1ccc(-c2cc(-c3ccccc3)cc(N(c3ccc(-c4cccc5ccccc45)cc3-c3ccccc3)c3cccc4c3c3ccccc3n4-c3ccccc3)c2)cc1 ; c1ccc(-c2cc(-c3ccccc3)cc(N(c3ccc(-c4ccc5ccccc5c4)cc3-c3ccccc3)c3cccc4c3c3ccccc3n4-c3ccccc3)c2)cc1 ; c1ccc(-c2cc(-c3ccccc3)cc(N(c3ccc(-c4cccc5c4oc4ccccc45)cc3-c3ccccc3)c3cccc4c3c3ccccc3n4-c3ccccc3)c2)cc1 ; c1ccc(-c2cc(-c3ccccc3)cc(N(c3ccc(-c4cccc5c4sc4ccccc45)cc3-c3ccccc3)c3cccc4c3c3ccccc3n4-c3ccccc3)c2)cc1]

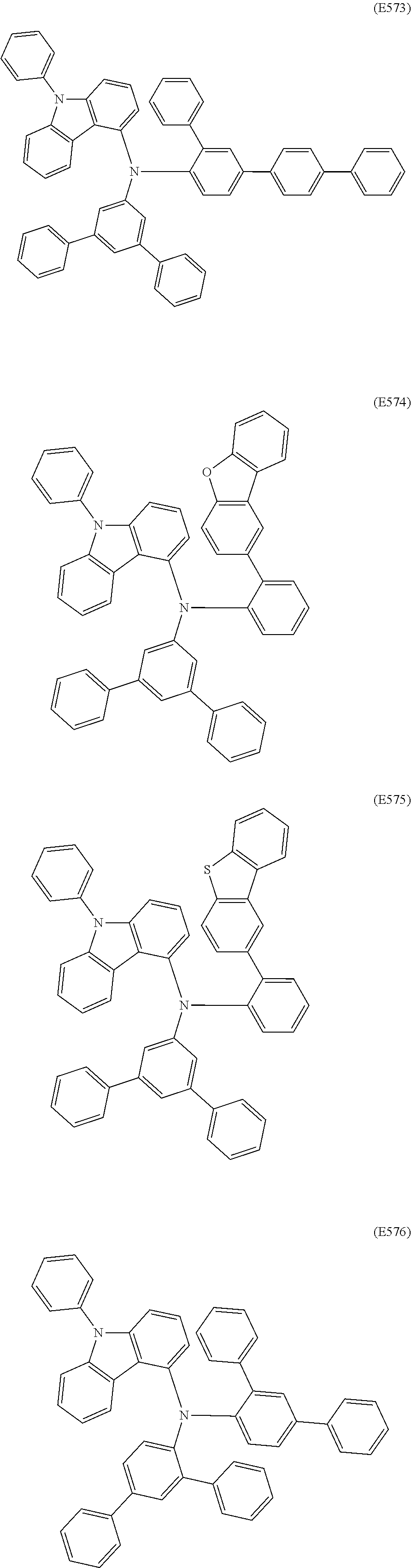 [SMILES: c1ccc(-c2ccc(-c3ccc(N(c4cc(-c5ccccc5)cc(-c5ccccc5)c4)c4cccc5c4c4ccccc4n5-c4ccccc4)c(-c4ccccc4)c3)cc2)cc1 ; c1ccc(-c2cc(-c3ccccc3)cc(N(c3ccccc3-c3ccc4oc5ccccc5c4c3)c3cccc4c3c3ccccc3n4-c3ccccc3)c2)cc1 ; c1ccc(-c2cc(-c3ccccc3)cc(N(c3ccccc3-c3ccc4sc5ccccc5c4c3)c3cccc4c3c3ccccc3n4-c3ccccc3)c2)cc1 ; c1ccc(-c2ccc(N(c3ccc(-c4ccccc4)cc3-c3ccccc3)c3cccc4c3c3ccccc3n4-c3ccccc3)c(-c3ccccc3)c2)cc1]

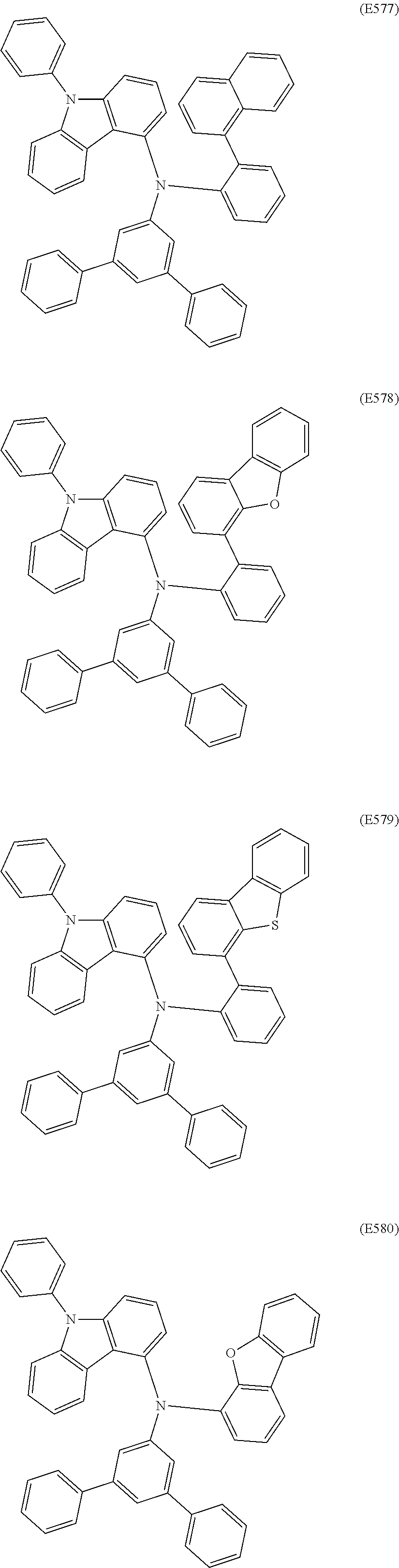 [SMILES: c1ccc(-c2cc(-c3ccccc3)cc(N(c3ccccc3-c3cccc4ccccc34)c3cccc4c3c3ccccc3n4-c3ccccc3)c2)cc1 ; c1ccc(-c2cc(-c3ccccc3)cc(N(c3ccccc3-c3cccc4c3oc3ccccc34)c3cccc4c3c3ccccc3n4-c3ccccc3)c2)cc1 ; c1ccc(-c2cc(-c3ccccc3)cc(N(c3ccccc3-c3cccc4c3sc3ccccc34)c3cccc4c3c3ccccc3n4-c3ccccc3)c2)cc1 ; c1ccc(-c2cc(-c3ccccc3)cc(N(c3cccc4c3oc3ccccc34)c3cccc4c3c3ccccc3n4-c3ccccc3)c2)cc1]

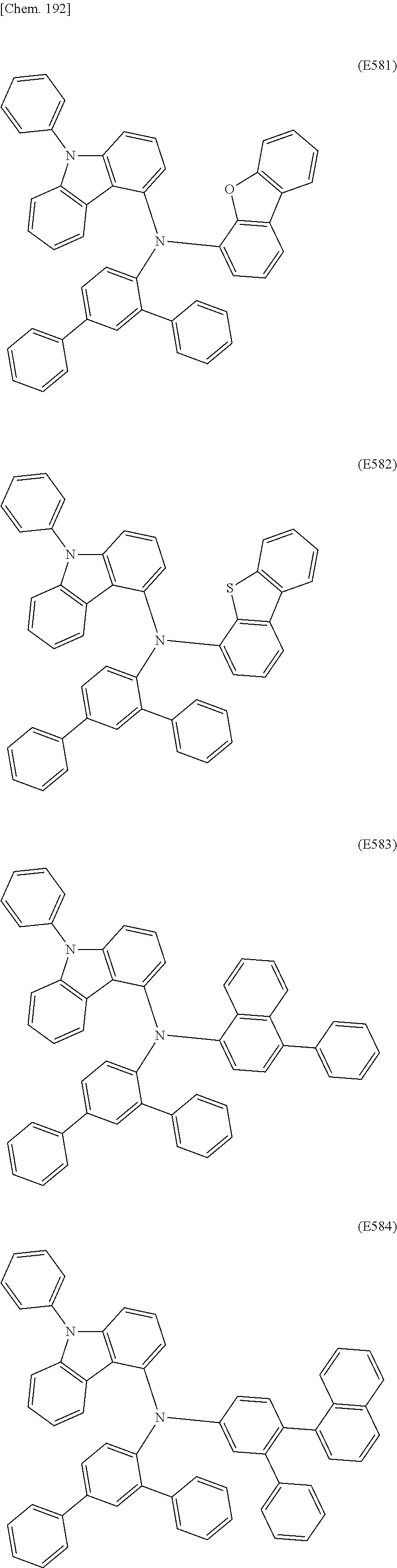 [SMILES: c1ccc(-c2ccc(N(c3cccc4c3oc3ccccc34)c3cccc4c3c3ccccc3n4-c3ccccc3)c(-c3ccccc3)c2)cc1 ; c1ccc(-c2ccc(N(c3cccc4c3sc3ccccc34)c3cccc4c3c3ccccc3n4-c3ccccc3)c(-c3ccccc3)c2)cc1 ; c1ccc(-c2ccc(N(c3ccc(-c4ccccc4)c4ccccc34)c3cccc4c3c3ccccc3n4-c3ccccc3)c(-c3ccccc3)c2)cc1 ; c1ccc(-c2ccc(N(c3ccc(-c4cccc5ccccc45)c(-c4ccccc4)c3)c3cccc4c3c3ccccc3n4-c3ccccc3)c(-c3ccccc3)c2)cc1]

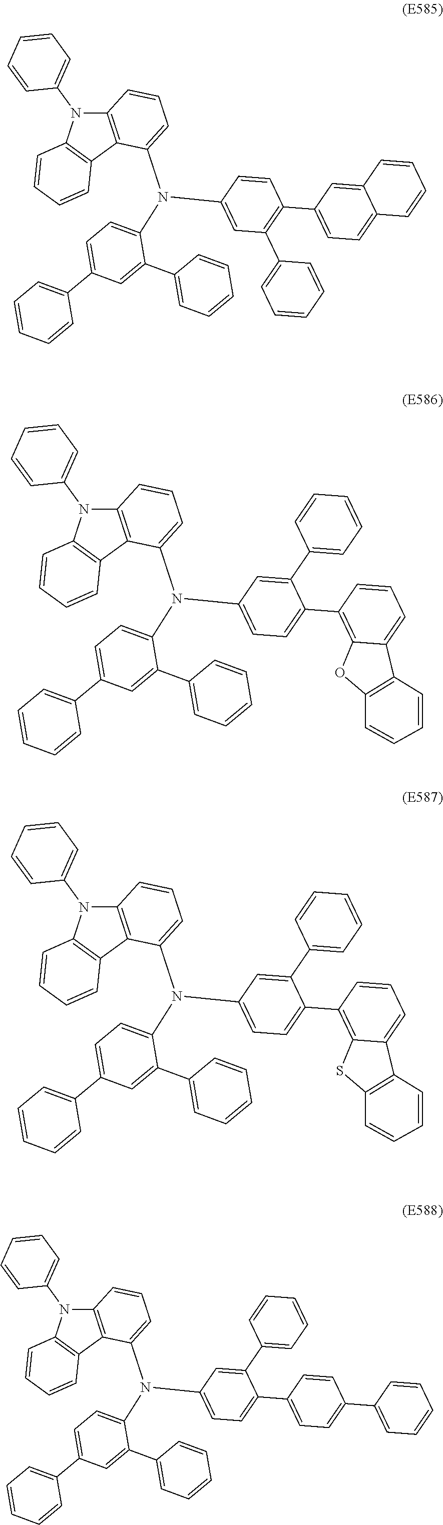 [SMILES: c1ccc(-c2ccc(N(c3ccc(-c4ccc5ccccc5c4)c(-c4ccccc4)c3)c3cccc4c3c3ccccc3n4-c3ccccc3)c(-c3ccccc3)c2)cc1 ; c1ccc(-c2ccc(N(c3ccc(-c4cccc5c4oc4ccccc45)c(-c4ccccc4)c3)c3cccc4c3c3ccccc3n4-c3ccccc3)c(-c3ccccc3)c2)cc1 ; c1ccc(-c2ccc(N(c3ccc(-c4cccc5c4sc4ccccc45)c(-c4ccccc4)c3)c3cccc4c3c3ccccc3n4-c3ccccc3)c(-c3ccccc3)c2)cc1 ; c1ccc(-c2ccc(-c3ccc(N(c4ccc(-c5ccccc5)cc4-c4ccccc4)c4cccc5c4c4ccccc4n5-c4ccccc4)cc3-c3ccccc3)cc2)cc1]

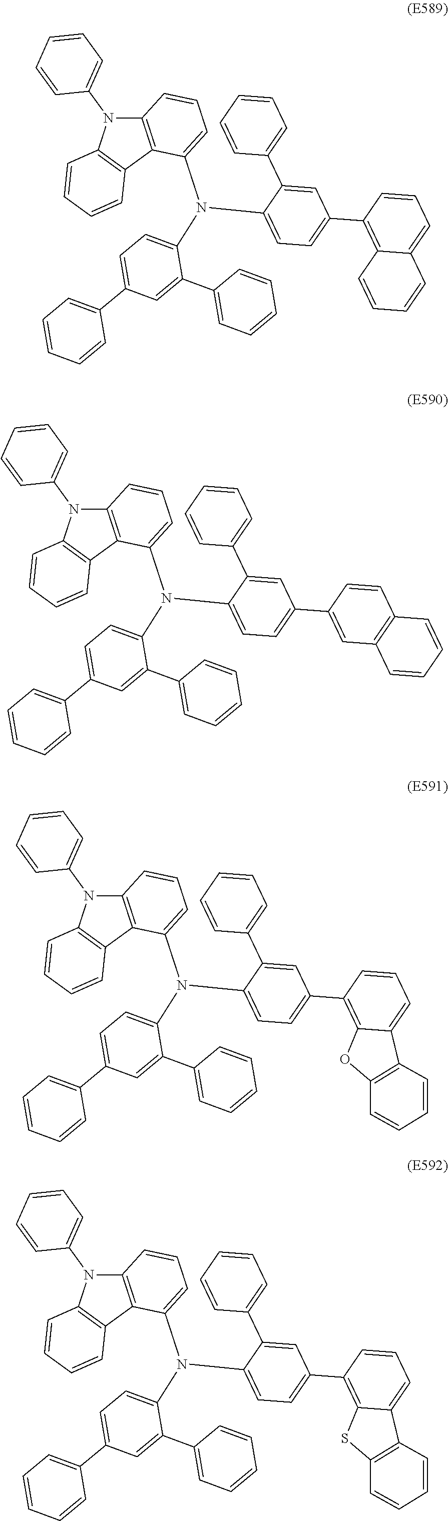 [SMILES: c1ccc(-c2ccc(N(c3ccc(-c4cccc5ccccc45)cc3-c3ccccc3)c3cccc4c3c3ccccc3n4-c3ccccc3)c(-c3ccccc3)c2)cc1 ; c1ccc(-c2ccc(N(c3ccc(-c4ccc5ccccc5c4)cc3-c3ccccc3)c3cccc4c3c3ccccc3n4-c3ccccc3)c(-c3ccccc3)c2)cc1 ; c1ccc(-c2ccc(N(c3ccc(-c4cccc5c4oc4ccccc45)cc3-c3ccccc3)c3cccc4c3c3ccccc3n4-c3ccccc3)c(-c3ccccc3)c2)cc1 ; c1ccc(-c2ccc(N(c3ccc(-c4cccc5c4sc4ccccc45)cc3-c3ccccc3)c3cccc4c3c3ccccc3n4-c3ccccc3)c(-c3ccccc3)c2)cc1]

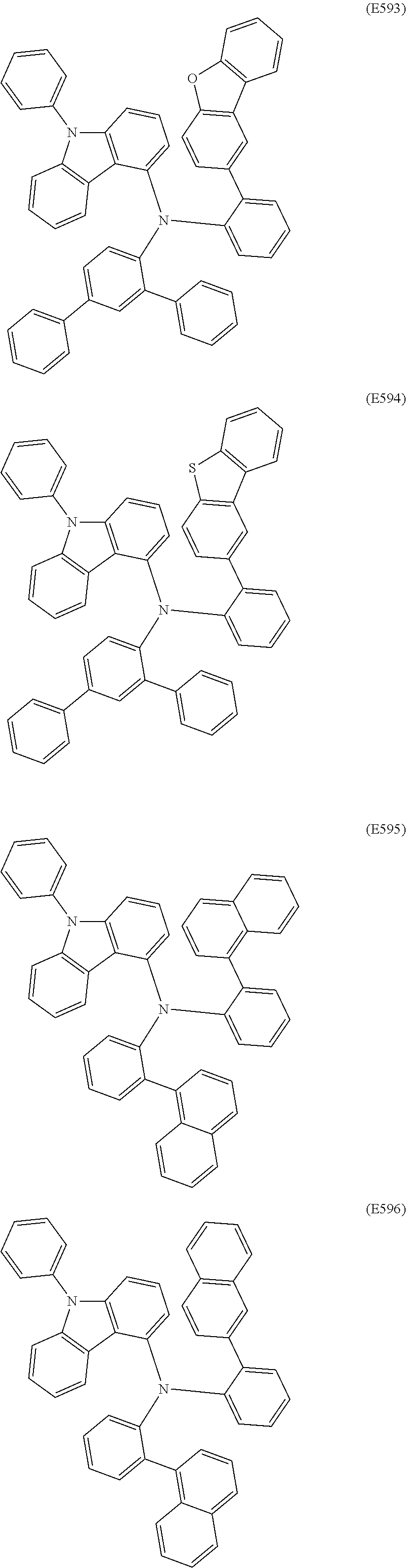

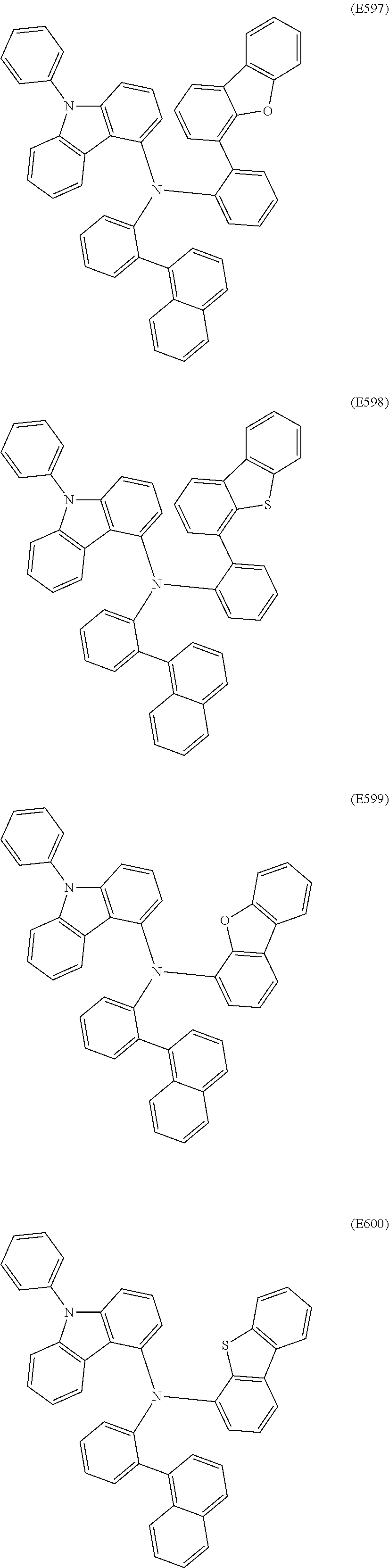 [SMILES: c1ccc(-n2c3ccccc3c3c(N(c4ccccc4-c4cccc5ccccc45)c4ccccc4-c4cccc5c4oc4ccccc45)cccc32)cc1 ; c1ccc(-n2c3ccccc3c3c(N(c4ccccc4-c4cccc5ccccc45)c4ccccc4-c4cccc5c4sc4ccccc45)cccc32)cc1 ; c1ccc(-n2c3ccccc3c3c(N(c4ccccc4-c4cccc5ccccc45)c4cccc5c4oc4ccccc45)cccc32)cc1 ; c1ccc(-n2c3ccccc3c3c(N(c4ccccc4-c4cccc5ccccc45)c4cccc5c4sc4ccccc45)cccc32)cc1]

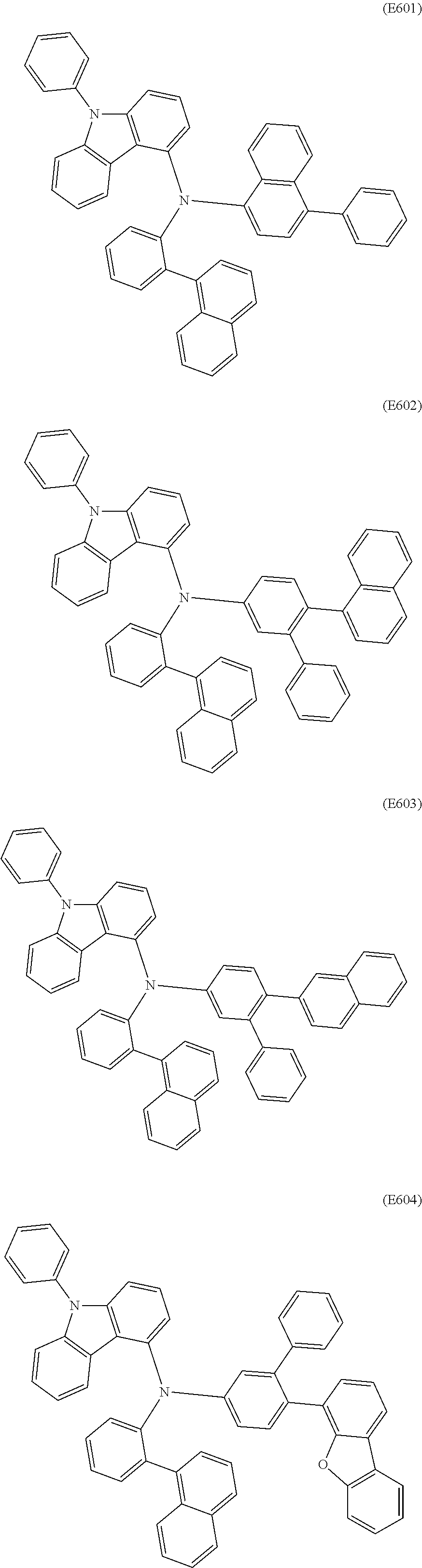 [SMILES: c1ccc(-c2ccc(N(c3ccccc3-c3cccc4ccccc34)c3cccc4c3c3ccccc3n4-c3ccccc3)c3ccccc23)cc1 ; c1ccc(-c2cc(N(c3ccccc3-c3cccc4ccccc34)c3cccc4c3c3ccccc3n4-c3ccccc3)ccc2-c2cccc3ccccc23)cc1 ; c1ccc(-c2cc(N(c3ccccc3-c3cccc4ccccc34)c3cccc4c3c3ccccc3n4-c3ccccc3)ccc2-c2ccc3ccccc3c2)cc1 ; c1ccc(-c2cc(N(c3ccccc3-c3cccc4ccccc34)c3cccc4c3c3ccccc3n4-c3ccccc3)ccc2-c2cccc3c2oc2ccccc23)cc1]

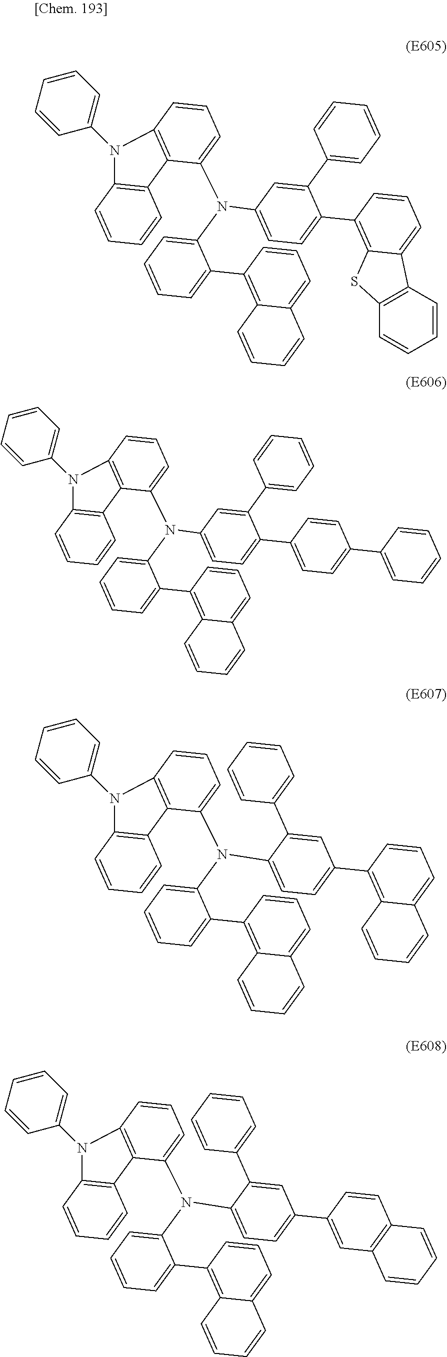 [SMILES: c1ccc(-c2cc(N(c3ccccc3-c3cccc4ccccc34)c3cccc4c3c3ccccc3n4-c3ccccc3)ccc2-c2cccc3c2sc2ccccc23)cc1 ; c1ccc(-c2ccc(-c3ccc(N(c4ccccc4-c4cccc5ccccc45)c4cccc5c4c4ccccc4n5-c4ccccc4)cc3-c3ccccc3)cc2)cc1 ; c1ccc(-c2cc(-c3cccc4ccccc34)ccc2N(c2ccccc2-c2cccc3ccccc23)c2cccc3c2c2ccccc2n3-c2ccccc2)cc1 ; c1ccc(-c2cc(-c3ccc4ccccc4c3)ccc2N(c2ccccc2-c2cccc3ccccc23)c2cccc3c2c2ccccc2n3-c2ccccc2)cc1]

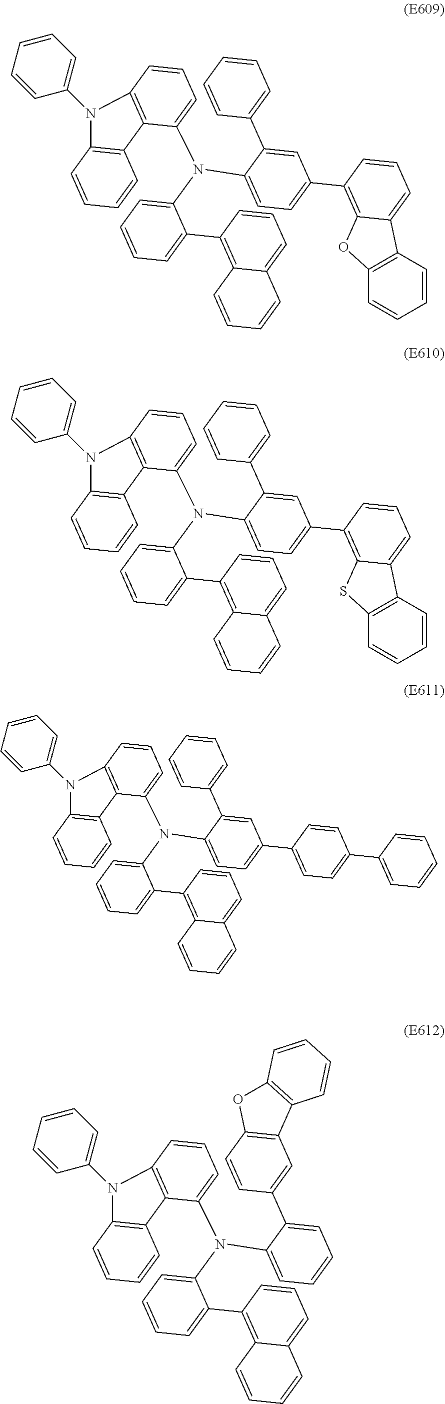 [SMILES: c1ccc(-c2cc(-c3cccc4c3oc3ccccc34)ccc2N(c2ccccc2-c2cccc3ccccc23)c2cccc3c2c2ccccc2n3-c2ccccc2)cc1 ; c1ccc(-c2cc(-c3cccc4c3sc3ccccc34)ccc2N(c2ccccc2-c2cccc3ccccc23)c2cccc3c2c2ccccc2n3-c2ccccc2)cc1 ; c1ccc(-c2ccc(-c3ccc(N(c4ccccc4-c4cccc5ccccc45)c4cccc5c4c4ccccc4n5-c4ccccc4)c(-c4ccccc4)c3)cc2)cc1 ; c1ccc(-n2c3ccccc3c3c(N(c4ccccc4-c4ccc5oc6ccccc6c5c4)c4ccccc4-c4cccc5ccccc45)cccc32)cc1]

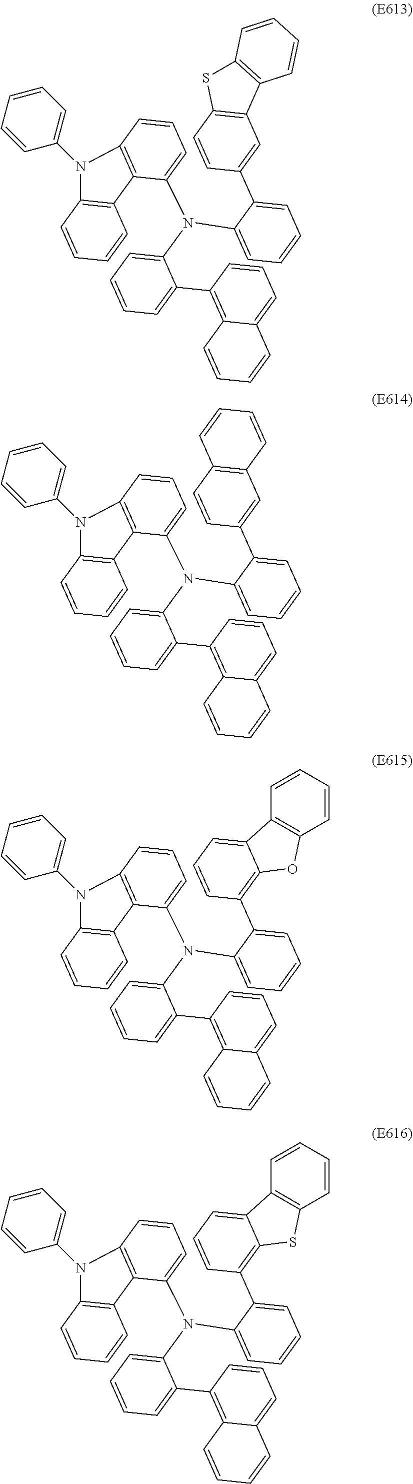

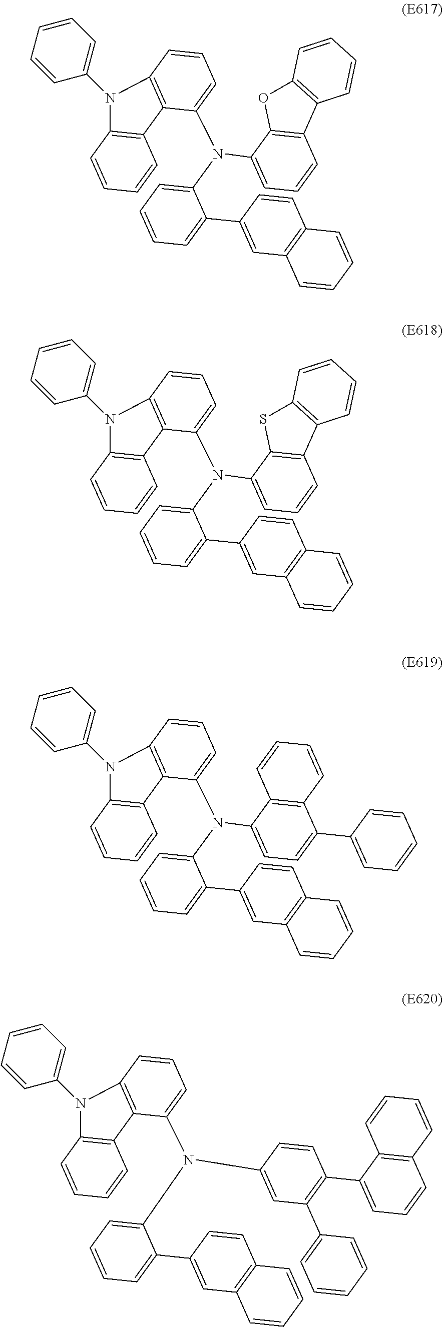 [SMILES: c1ccc(-n2c3ccccc3c3c(N(c4ccccc4-c4ccc5ccccc5c4)c4cccc5c4oc4ccccc45)cccc32)cc1 ; c1ccc(-n2c3ccccc3c3c(N(c4ccccc4-c4ccc5ccccc5c4)c4cccc5c4sc4ccccc45)cccc32)cc1 ; c1ccc(-c2ccc(N(c3ccccc3-c3ccc4ccccc4c3)c3cccc4c3c3ccccc3n4-c3ccccc3)c3ccccc23)cc1 ; c1ccc(-c2cc(N(c3ccccc3-c3ccc4ccccc4c3)c3cccc4c3c3ccccc3n4-c3ccccc3)ccc2-c2cccc3ccccc23)cc1]

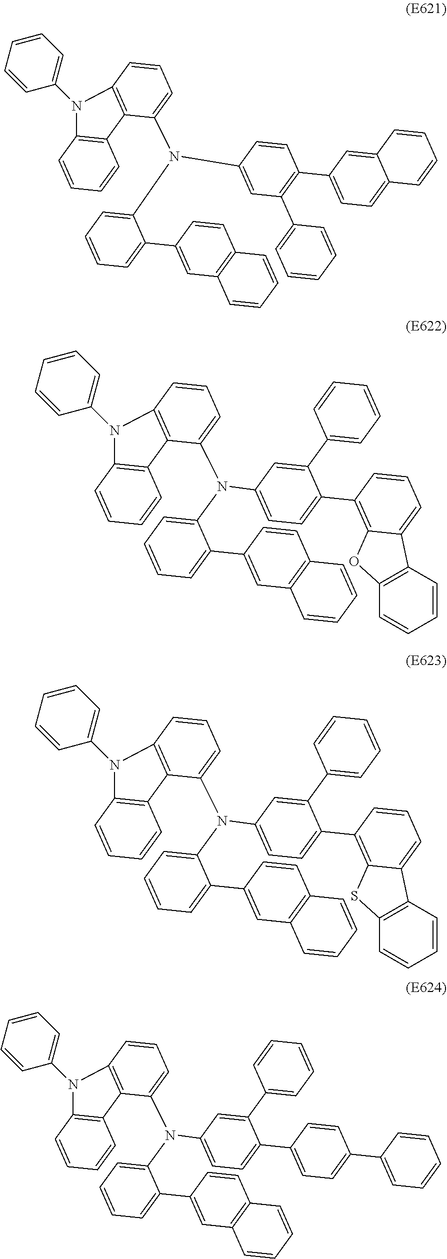 [SMILES: c1ccc(-c2cc(N(c3ccccc3-c3ccc4ccccc4c3)c3cccc4c3c3ccccc3n4-c3ccccc3)ccc2-c2ccc3ccccc3c2)cc1 ; c1ccc(-c2cc(N(c3ccccc3-c3ccc4ccccc4c3)c3cccc4c3c3ccccc3n4-c3ccccc3)ccc2-c2cccc3c2oc2ccccc23)cc1 ; c1ccc(-c2cc(N(c3ccccc3-c3ccc4ccccc4c3)c3cccc4c3c3ccccc3n4-c3ccccc3)ccc2-c2cccc3c2sc2ccccc23)cc1 ; c1ccc(-c2ccc(-c3ccc(N(c4ccccc4-c4ccc5ccccc5c4)c4cccc5c4c4ccccc4n5-c4ccccc4)cc3-c3ccccc3)cc2)cc1]

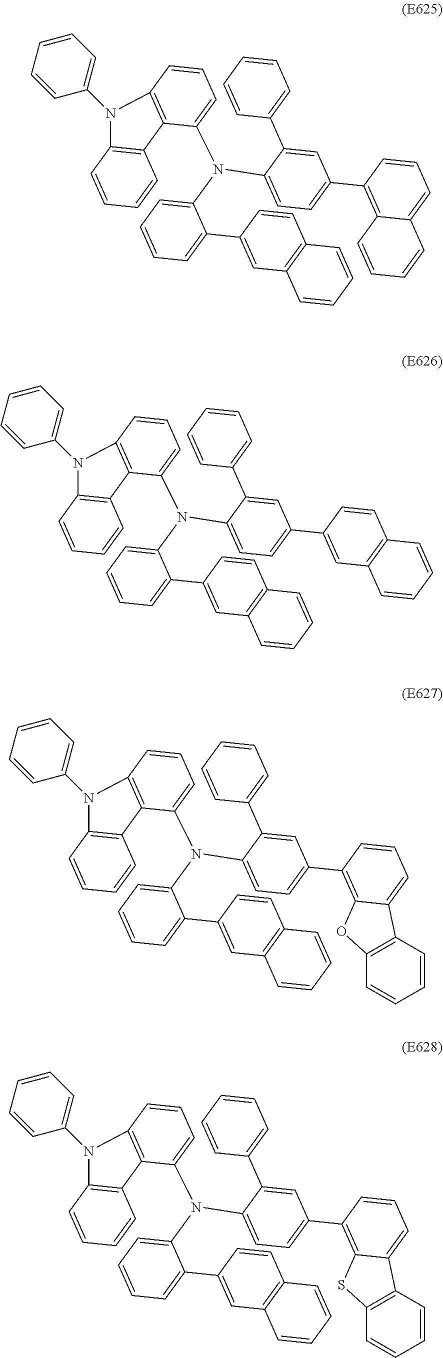 [SMILES: c1ccc(-c2cc(-c3cccc4ccccc34)ccc2N(c2ccccc2-c2ccc3ccccc3c2)c2cccc3c2c2ccccc2n3-c2ccccc2)cc1 ; c1ccc(-c2cc(-c3ccc4ccccc4c3)ccc2N(c2ccccc2-c2ccc3ccccc3c2)c2cccc3c2c2ccccc2n3-c2ccccc2)cc1 ; c1ccc(-c2cc(-c3cccc4c3oc3ccccc34)ccc2N(c2ccccc2-c2ccc3ccccc3c2)c2cccc3c2c2ccccc2n3-c2ccccc2)cc1 ; c1ccc(-c2cc(-c3cccc4c3sc3ccccc34)ccc2N(c2ccccc2-c2ccc3ccccc3c2)c2cccc3c2c2ccccc2n3-c2ccccc2)cc1]

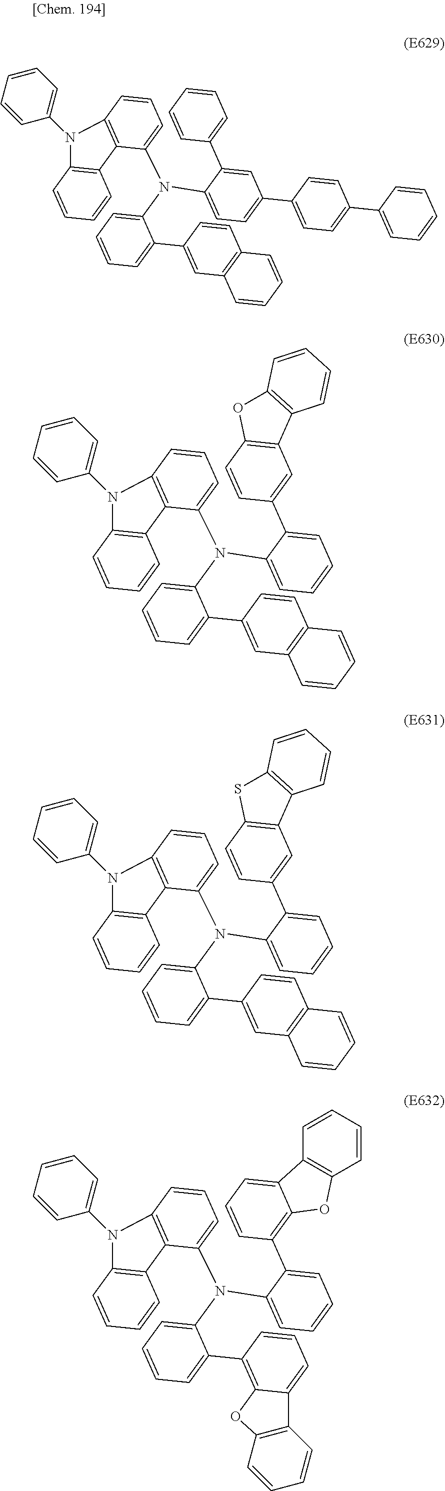

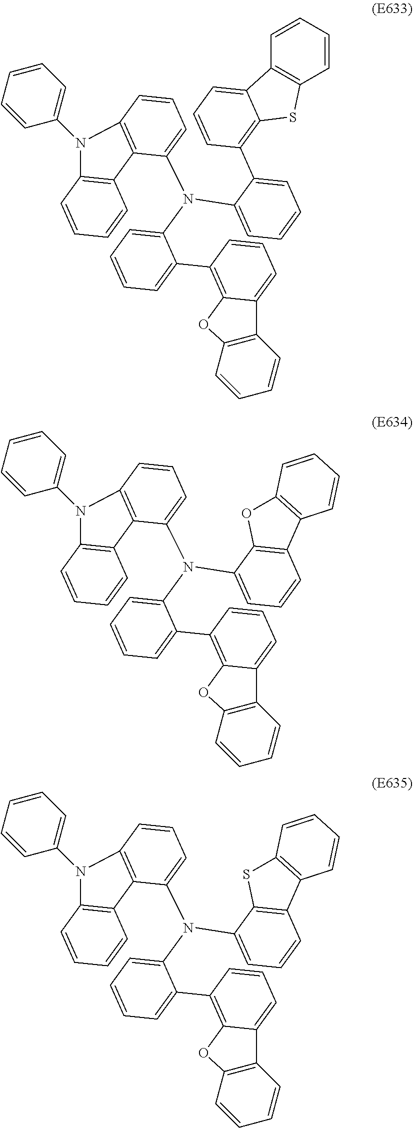 [SMILES: c1ccc(-n2c3ccccc3c3c(N(c4ccccc4-c4cccc5c4oc4ccccc45)c4ccccc4-c4cccc5c4sc4ccccc45)cccc32)cc1 ; c1ccc(-n2c3ccccc3c3c(N(c4ccccc4-c4cccc5c4oc4ccccc45)c4cccc5c4oc4ccccc45)cccc32)cc1 ; c1ccc(-n2c3ccccc3c3c(N(c4ccccc4-c4cccc5c4oc4ccccc45)c4cccc5c4sc4ccccc45)cccc32)cc1]

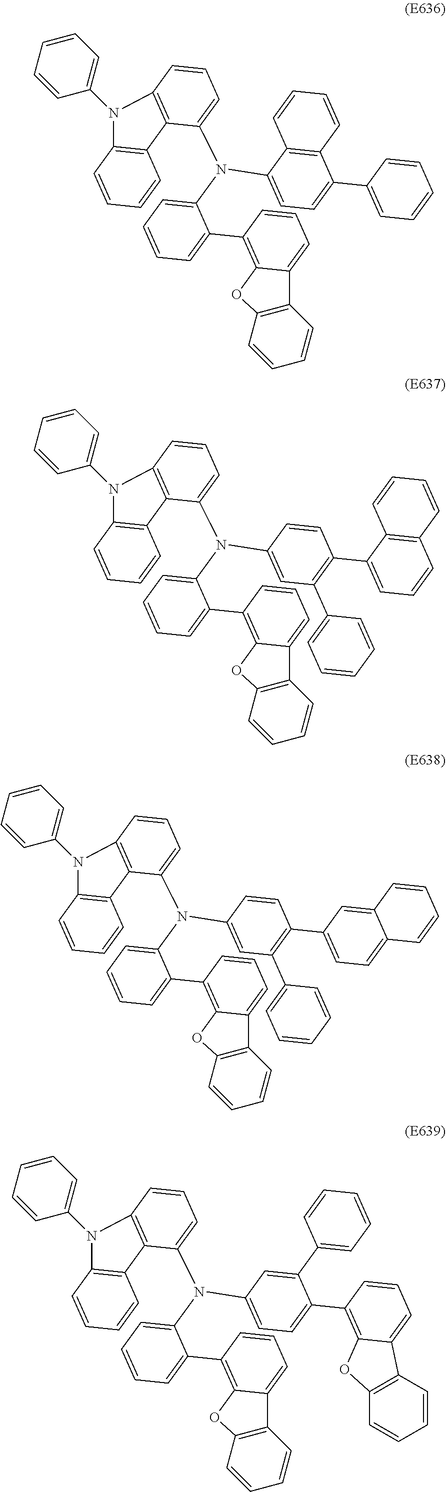 [SMILES: c1ccc(-c2ccc(N(c3ccccc3-c3cccc4c3oc3ccccc34)c3cccc4c3c3ccccc3n4-c3ccccc3)c3ccccc23)cc1 ; c1ccc(-c2cc(N(c3ccccc3-c3cccc4c3oc3ccccc34)c3cccc4c3c3ccccc3n4-c3ccccc3)ccc2-c2cccc3ccccc23)cc1 ; c1ccc(-c2cc(N(c3ccccc3-c3cccc4c3oc3ccccc34)c3cccc4c3c3ccccc3n4-c3ccccc3)ccc2-c2ccc3ccccc3c2)cc1 ; c1ccc(-c2cc(N(c3ccccc3-c3cccc4c3oc3ccccc34)c3cccc4c3c3ccccc3n4-c3ccccc3)ccc2-c2cccc3c2oc2ccccc23)cc1]

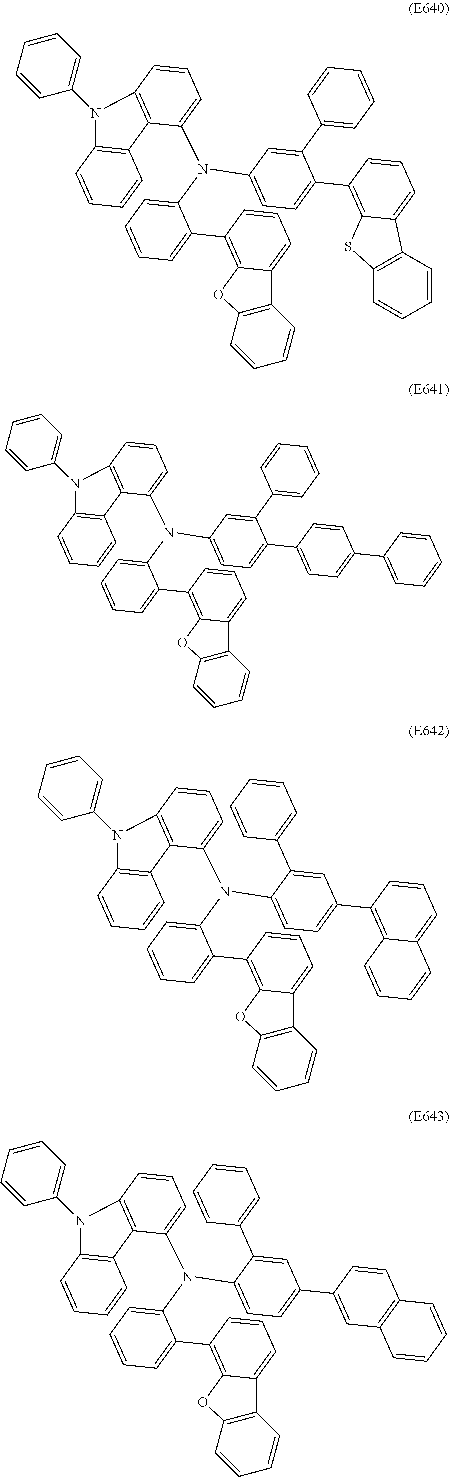 [SMILES: c1ccc(-c2cc(N(c3ccccc3-c3cccc4c3oc3ccccc34)c3cccc4c3c3ccccc3n4-c3ccccc3)ccc2-c2cccc3c2sc2ccccc23)cc1 ; c1ccc(-c2ccc(-c3ccc(N(c4ccccc4-c4cccc5c4oc4ccccc45)c4cccc5c4c4ccccc4n5-c4ccccc4)cc3-c3ccccc3)cc2)cc1 ; c1ccc(-c2cc(-c3cccc4ccccc34)ccc2N(c2ccccc2-c2cccc3c2oc2ccccc23)c2cccc3c2c2ccccc2n3-c2ccccc2)cc1 ; c1ccc(-c2cc(-c3ccc4ccccc4c3)ccc2N(c2ccccc2-c2cccc3c2oc2ccccc23)c2cccc3c2c2ccccc2n3-c2ccccc2)cc1]

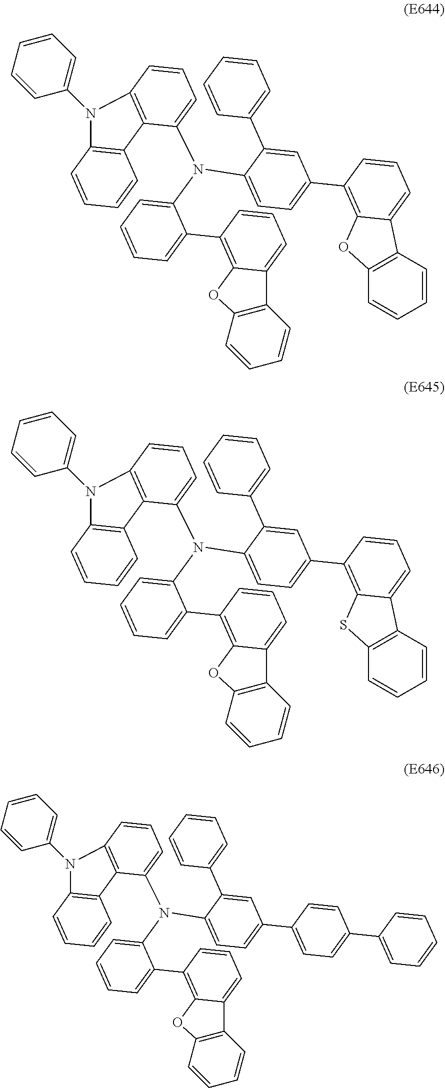 [SMILES: c1ccc(-c2cc(-c3cccc4c3oc3ccccc34)ccc2N(c2ccccc2-c2cccc3c2oc2ccccc23)c2cccc3c2c2ccccc2n3-c2ccccc2)cc1 ; c1ccc(-c2cc(-c3cccc4c3sc3ccccc34)ccc2N(c2ccccc2-c2cccc3c2oc2ccccc23)c2cccc3c2c2ccccc2n3-c2ccccc2)cc1 ; c1ccc(-c2ccc(-c3ccc(N(c4ccccc4-c4cccc5c4oc4ccccc45)c4cccc5c4c4ccccc4n5-c4ccccc4)c(-c4ccccc4)c3)cc2)cc1]

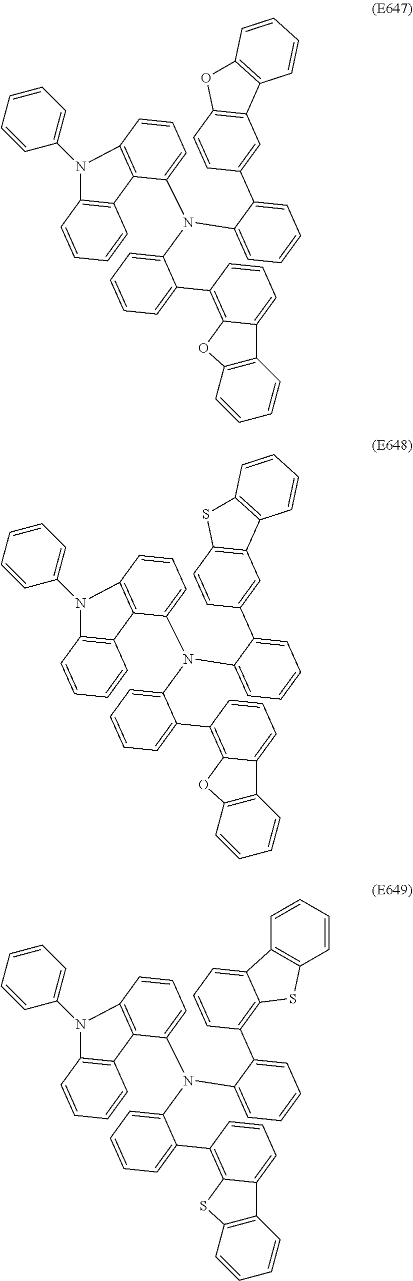

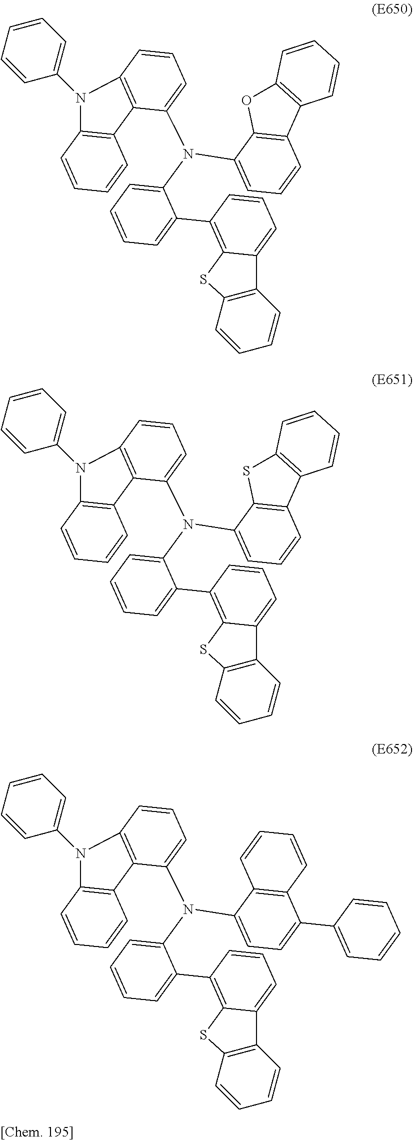 [SMILES: c1ccc(-n2c3ccccc3c3c(N(c4ccccc4-c4cccc5c4sc4ccccc45)c4cccc5c4oc4ccccc45)cccc32)cc1 ; c1ccc(-n2c3ccccc3c3c(N(c4ccccc4-c4cccc5c4sc4ccccc45)c4cccc5c4sc4ccccc45)cccc32)cc1 ; c1ccc(-c2ccc(N(c3ccccc3-c3cccc4c3sc3ccccc34)c3cccc4c3c3ccccc3n4-c3ccccc3)c3ccccc23)cc1]

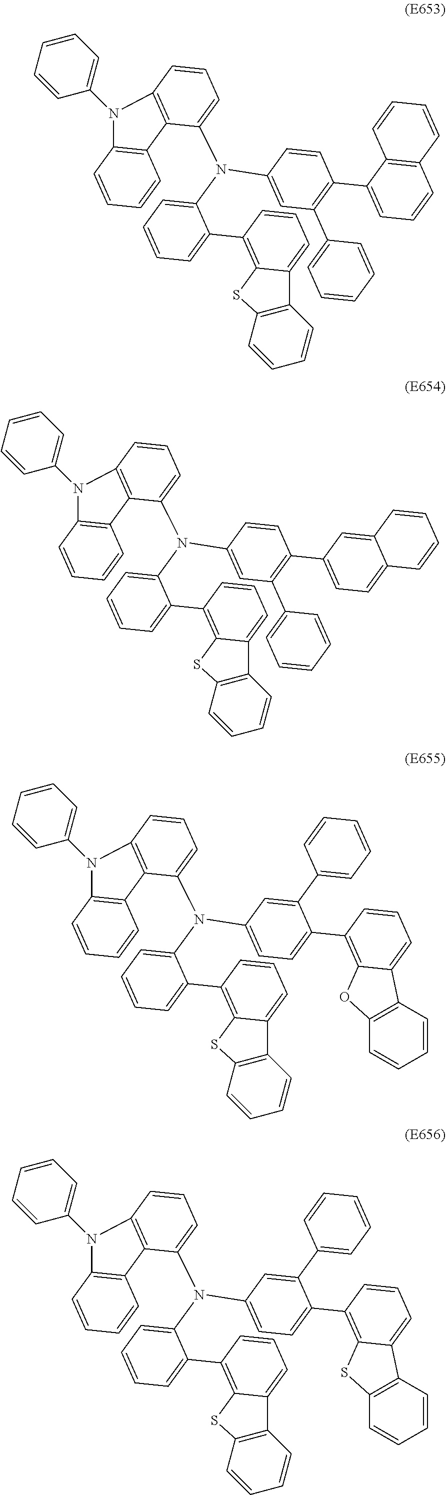 [SMILES: c1ccc(-c2cc(N(c3ccccc3-c3cccc4c3sc3ccccc34)c3cccc4c3c3ccccc3n4-c3ccccc3)ccc2-c2cccc3ccccc23)cc1 ; c1ccc(-c2cc(N(c3ccccc3-c3cccc4c3sc3ccccc34)c3cccc4c3c3ccccc3n4-c3ccccc3)ccc2-c2ccc3ccccc3c2)cc1 ; c1ccc(-c2cc(N(c3ccccc3-c3cccc4c3sc3ccccc34)c3cccc4c3c3ccccc3n4-c3ccccc3)ccc2-c2cccc3c2oc2ccccc23)cc1 ; c1ccc(-c2cc(N(c3ccccc3-c3cccc4c3sc3ccccc34)c3cccc4c3c3ccccc3n4-c3ccccc3)ccc2-c2cccc3c2sc2ccccc23)cc1]

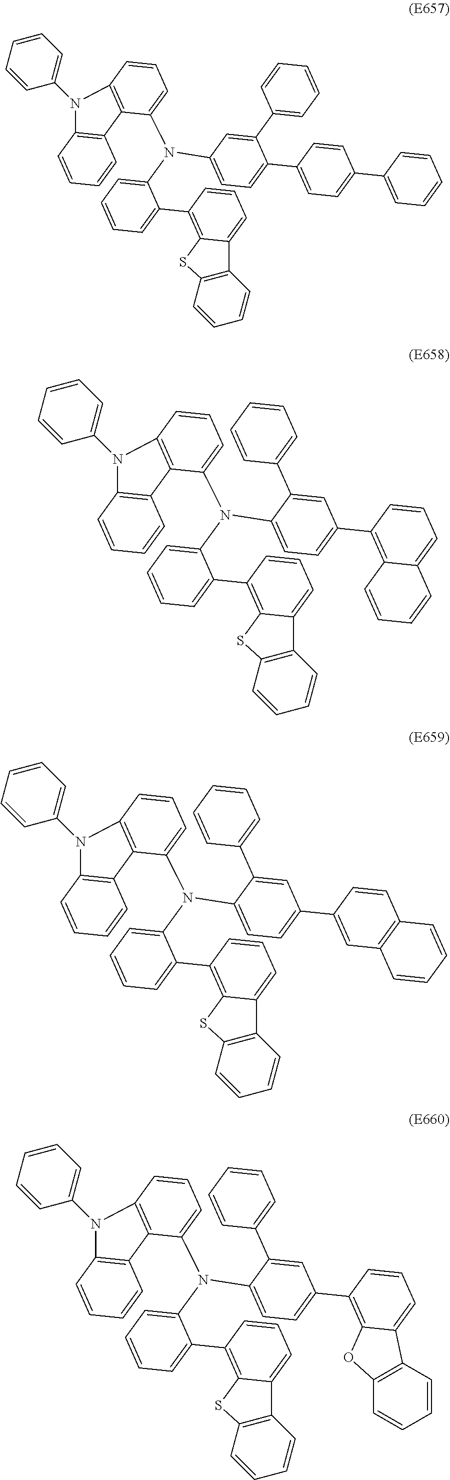 [SMILES: c1ccc(-c2ccc(-c3ccc(N(c4ccccc4-c4cccc5c4sc4ccccc45)c4cccc5c4c4ccccc4n5-c4ccccc4)cc3-c3ccccc3)cc2)cc1 ; c1ccc(-c2cc(-c3cccc4ccccc34)ccc2N(c2ccccc2-c2cccc3c2sc2ccccc23)c2cccc3c2c2ccccc2n3-c2ccccc2)cc1 ; c1ccc(-c2cc(-c3ccc4ccccc4c3)ccc2N(c2ccccc2-c2cccc3c2sc2ccccc23)c2cccc3c2c2ccccc2n3-c2ccccc2)cc1 ; c1ccc(-c2cc(-c3cccc4c3oc3ccccc34)ccc2N(c2ccccc2-c2cccc3c2sc2ccccc23)c2cccc3c2c2ccccc2n3-c2ccccc2)cc1]

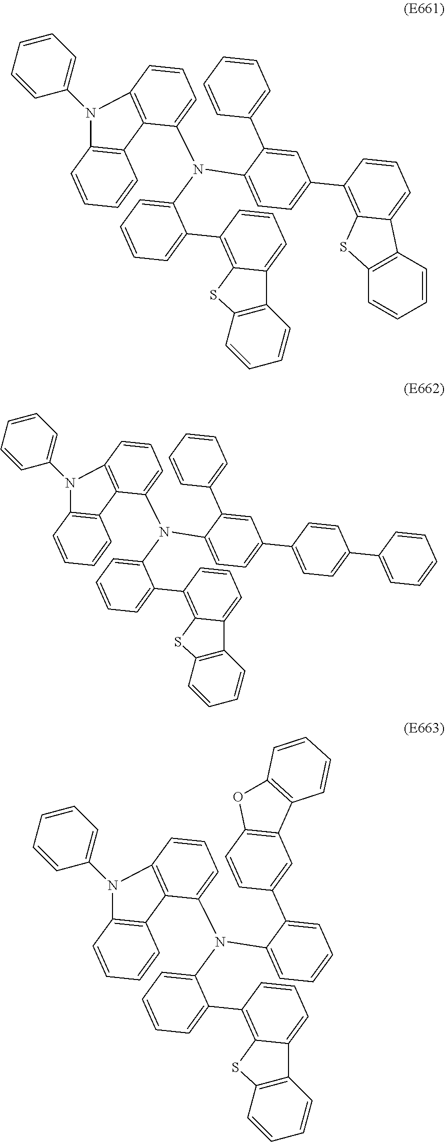 [SMILES: c1ccc(-c2cc(-c3cccc4c3sc3ccccc34)ccc2N(c2ccccc2-c2cccc3c2sc2ccccc23)c2cccc3c2c2ccccc2n3-c2ccccc2)cc1 ; c1ccc(-c2ccc(-c3ccc(N(c4ccccc4-c4cccc5c4sc4ccccc45)c4cccc5c4c4ccccc4n5-c4ccccc4)c(-c4ccccc4)c3)cc2)cc1 ; c1ccc(-n2c3ccccc3c3c(N(c4ccccc4-c4ccc5oc6ccccc6c5c4)c4ccccc4-c4cccc5c4sc4ccccc45)cccc32)cc1]

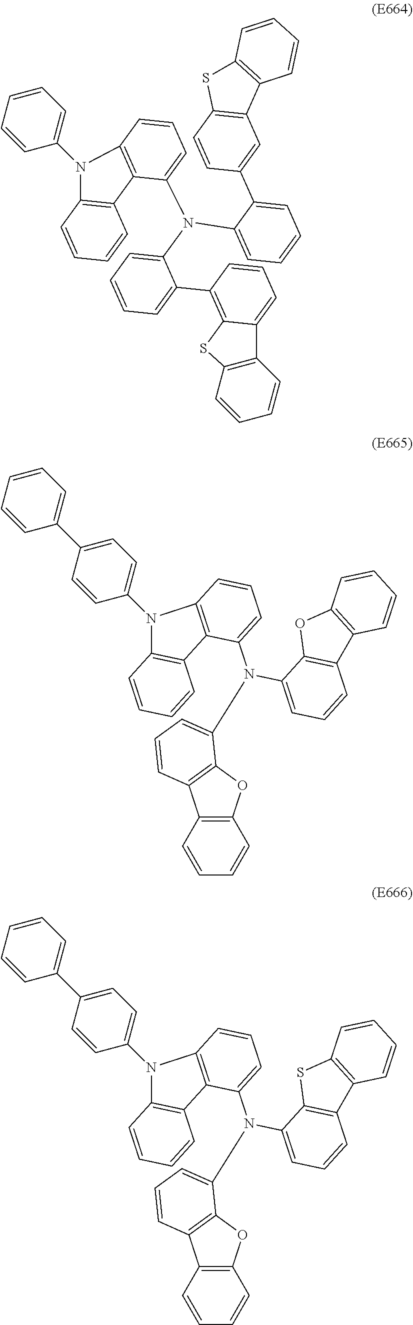 [SMILES: c1ccc(-n2c3ccccc3c3c(N(c4ccccc4-c4ccc5sc6ccccc6c5c4)c4ccccc4-c4cccc5c4sc4ccccc45)cccc32)cc1 ; c1ccc(-c2ccc(-n3c4ccccc4c4c(N(c5cccc6c5oc5ccccc56)c5cccc6c5oc5ccccc56)cccc43)cc2)cc1 ; c1ccc(-c2ccc(-n3c4ccccc4c4c(N(c5cccc6c5oc5ccccc56)c5cccc6c5sc5ccccc56)cccc43)cc2)cc1]

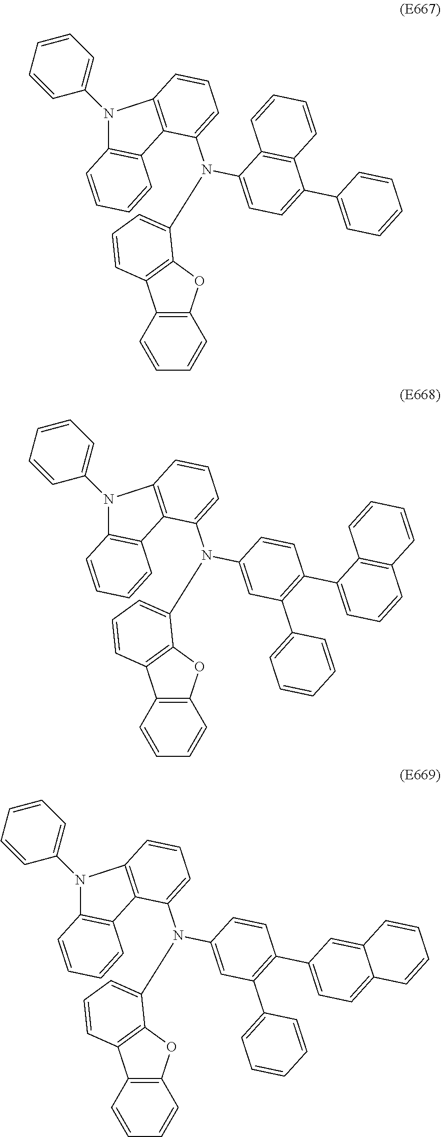 [SMILES: c1ccc(-c2ccc(N(c3cccc4c3oc3ccccc34)c3cccc4c3c3ccccc3n4-c3ccccc3)c3ccccc23)cc1 ; c1ccc(-c2cc(N(c3cccc4c3oc3ccccc34)c3cccc4c3c3ccccc3n4-c3ccccc3)ccc2-c2cccc3ccccc23)cc1 ; c1ccc(-c2cc(N(c3cccc4c3oc3ccccc34)c3cccc4c3c3ccccc3n4-c3ccccc3)ccc2-c2ccc3ccccc3c2)cc1]

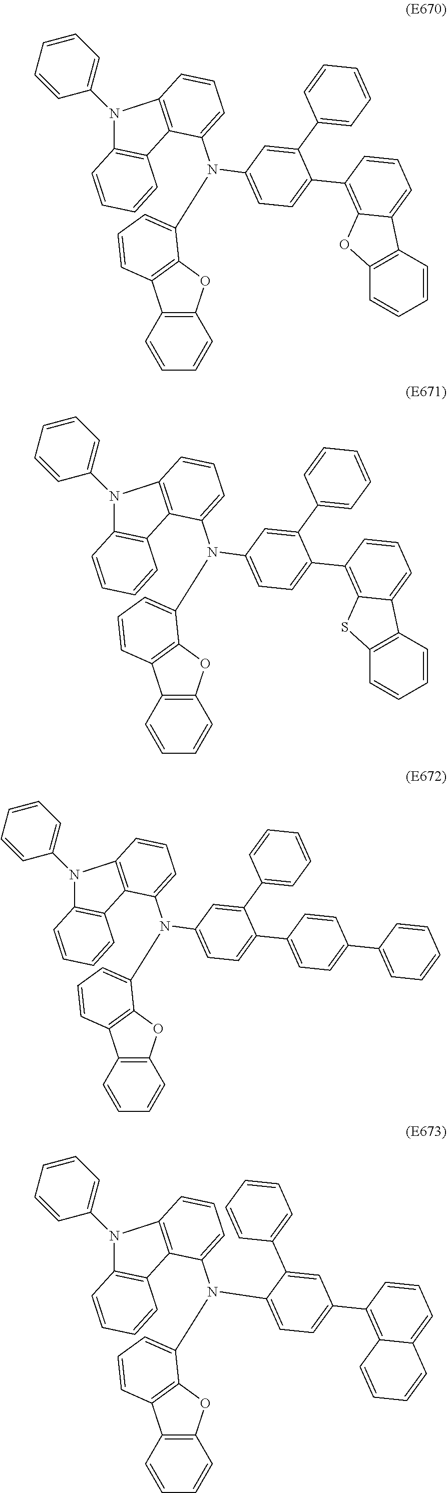 [SMILES: c1ccc(-c2cc(N(c3cccc4c3oc3ccccc34)c3cccc4c3c3ccccc3n4-c3ccccc3)ccc2-c2cccc3c2oc2ccccc23)cc1 ; c1ccc(-c2cc(N(c3cccc4c3oc3ccccc34)c3cccc4c3c3ccccc3n4-c3ccccc3)ccc2-c2cccc3c2sc2ccccc23)cc1 ; c1ccc(-c2ccc(-c3ccc(N(c4cccc5c4oc4ccccc45)c4cccc5c4c4ccccc4n5-c4ccccc4)cc3-c3ccccc3)cc2)cc1 ; c1ccc(-c2cc(-c3cccc4ccccc34)ccc2N(c2cccc3c2oc2ccccc23)c2cccc3c2c2ccccc2n3-c2ccccc2)cc1]

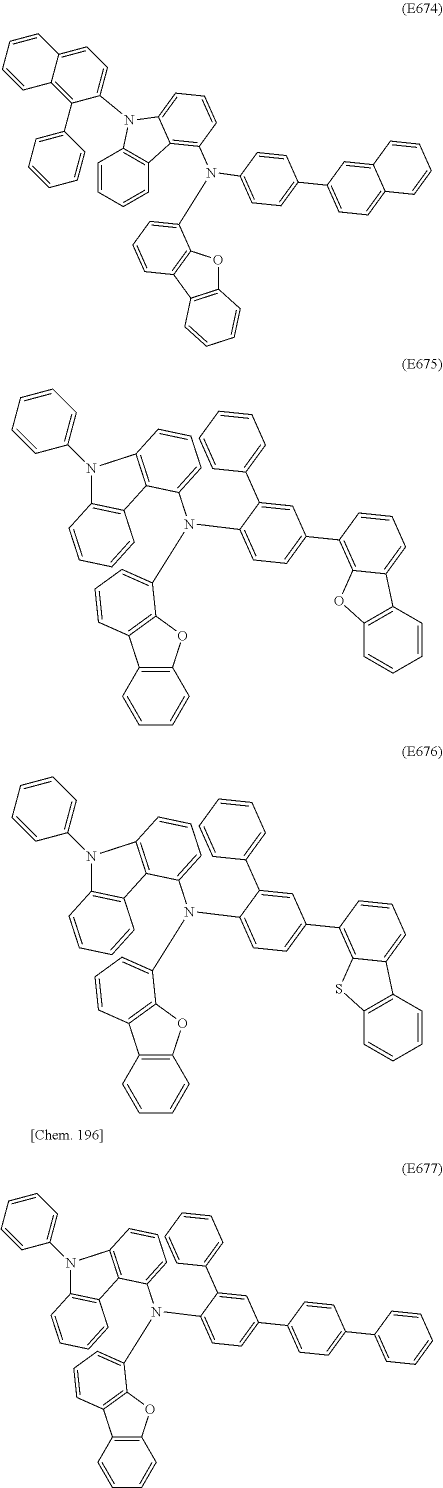 [SMILES: c1ccc(-c2c(-n3c4ccccc4c4c(N(c5ccc(-c6ccc7ccccc7c6)cc5)c5cccc6c5oc5ccccc56)cccc43)ccc3ccccc23)cc1 ; c1ccc(-c2cc(-c3cccc4c3oc3ccccc34)ccc2N(c2cccc3c2oc2ccccc23)c2cccc3c2c2ccccc2n3-c2ccccc2)cc1 ; c1ccc(-c2cc(-c3cccc4c3sc3ccccc34)ccc2N(c2cccc3c2oc2ccccc23)c2cccc3c2c2ccccc2n3-c2ccccc2)cc1 ; c1ccc(-c2ccc(-c3ccc(N(c4cccc5c4oc4ccccc45)c4cccc5c4c4ccccc4n5-c4ccccc4)c(-c4ccccc4)c3)cc2)cc1]

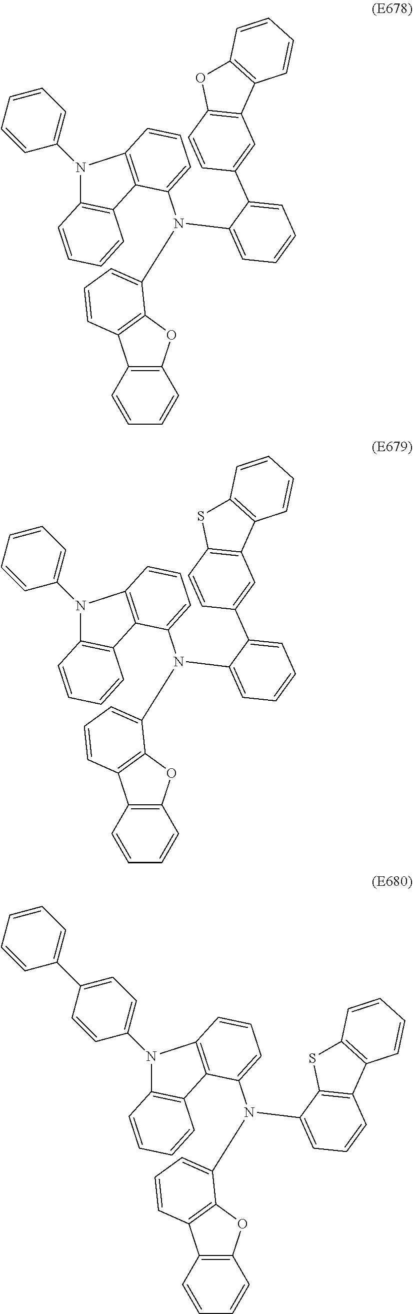 [SMILES: c1ccc(-n2c3ccccc3c3c(N(c4ccccc4-c4ccc5oc6ccccc6c5c4)c4cccc5c4oc4ccccc45)cccc32)cc1 ; c1ccc(-n2c3ccccc3c3c(N(c4ccccc4-c4ccc5sc6ccccc6c5c4)c4cccc5c4oc4ccccc45)cccc32)cc1 ; c1ccc(-c2ccc(-n3c4ccccc4c4c(N(c5cccc6c5oc5ccccc56)c5cccc6c5sc5ccccc56)cccc43)cc2)cc1]

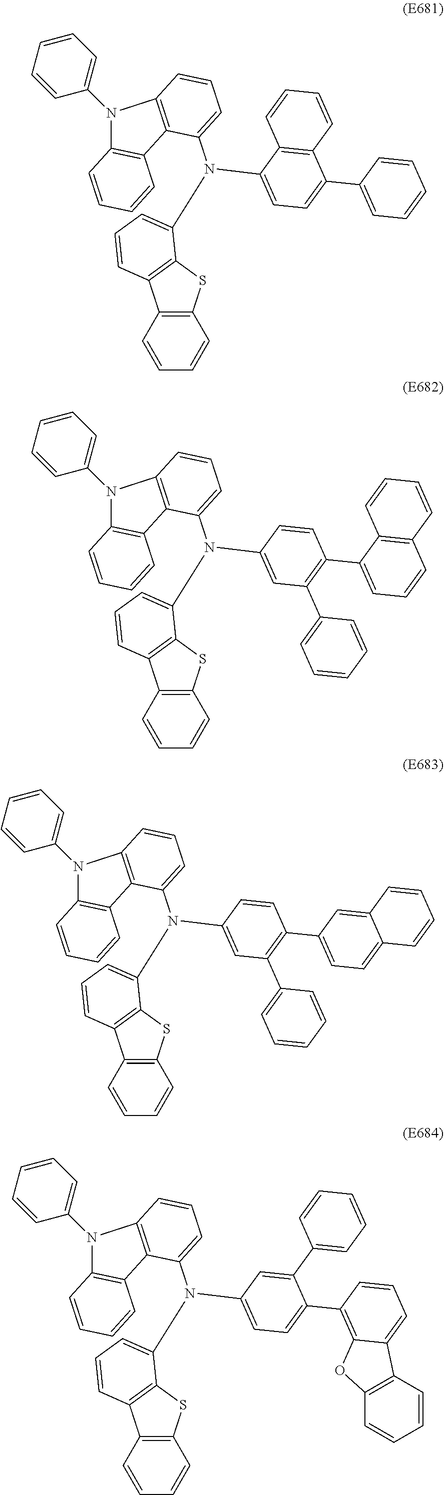 [SMILES: c1ccc(-c2ccc(N(c3cccc4c3sc3ccccc34)c3cccc4c3c3ccccc3n4-c3ccccc3)c3ccccc23)cc1 ; c1ccc(-c2cc(N(c3cccc4c3sc3ccccc34)c3cccc4c3c3ccccc3n4-c3ccccc3)ccc2-c2cccc3ccccc23)cc1 ; c1ccc(-c2cc(N(c3cccc4c3sc3ccccc34)c3cccc4c3c3ccccc3n4-c3ccccc3)ccc2-c2ccc3ccccc3c2)cc1 ; c1ccc(-c2cc(N(c3cccc4c3sc3ccccc34)c3cccc4c3c3ccccc3n4-c3ccccc3)ccc2-c2cccc3c2oc2ccccc23)cc1]

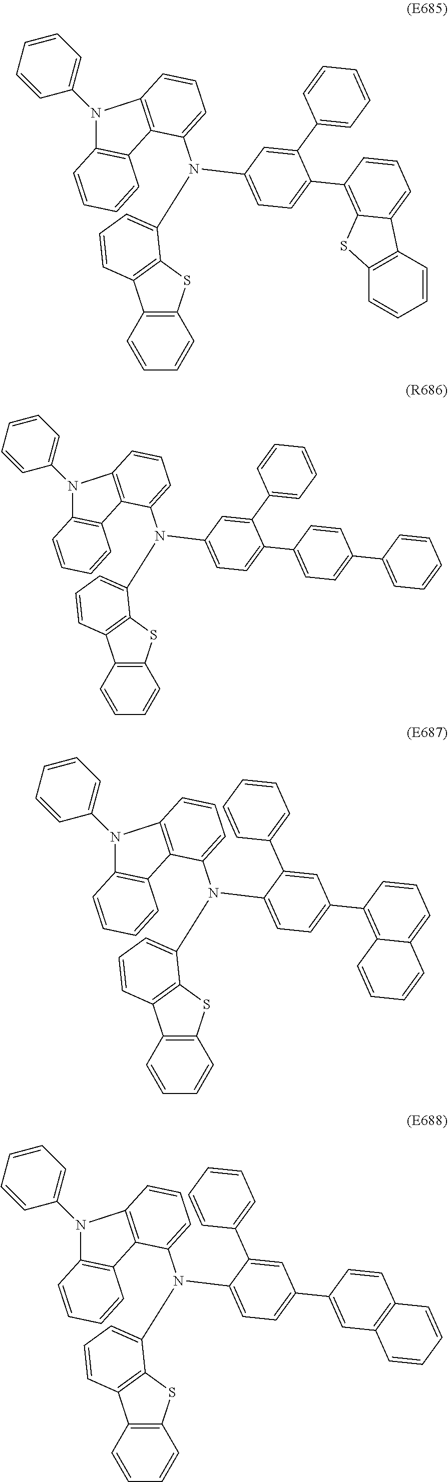 [SMILES: c1ccc(-c2cc(N(c3cccc4c3sc3ccccc34)c3cccc4c3c3ccccc3n4-c3ccccc3)ccc2-c2cccc3c2sc2ccccc23)cc1 ; c1ccc(-c2ccc(-c3ccc(N(c4cccc5c4sc4ccccc45)c4cccc5c4c4ccccc4n5-c4ccccc4)cc3-c3ccccc3)cc2)cc1 ; c1ccc(-c2cc(-c3cccc4ccccc34)ccc2N(c2cccc3c2sc2ccccc23)c2cccc3c2c2ccccc2n3-c2ccccc2)cc1 ; c1ccc(-c2cc(-c3ccc4ccccc4c3)ccc2N(c2cccc3c2sc2ccccc23)c2cccc3c2c2ccccc2n3-c2ccccc2)cc1]

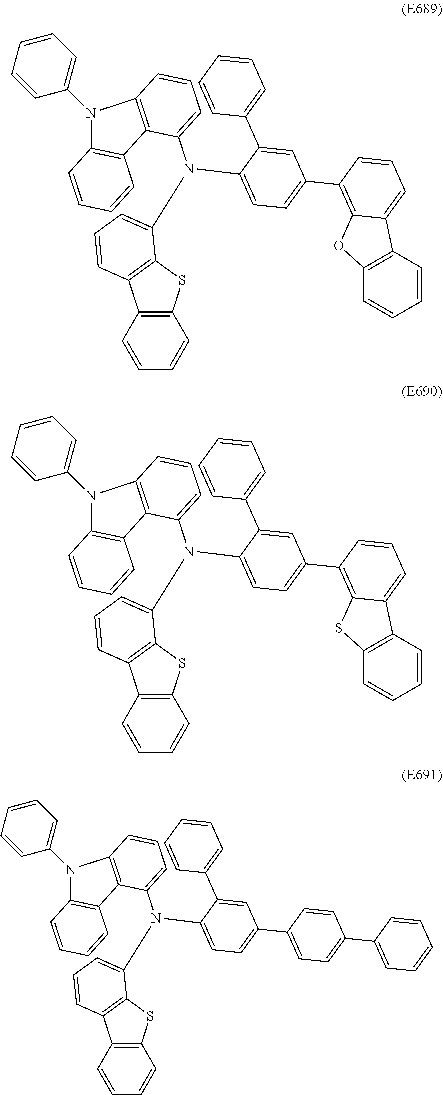 [SMILES: c1ccc(-c2cc(-c3cccc4c3oc3ccccc34)ccc2N(c2cccc3c2sc2ccccc23)c2cccc3c2c2ccccc2n3-c2ccccc2)cc1 ; c1ccc(-c2cc(-c3cccc4c3sc3ccccc34)ccc2N(c2cccc3c2sc2ccccc23)c2cccc3c2c2ccccc2n3-c2ccccc2)cc1 ; c1ccc(-c2ccc(-c3ccc(N(c4cccc5c4sc4ccccc45)c4cccc5c4c4ccccc4n5-c4ccccc4)c(-c4ccccc4)c3)cc2)cc1]

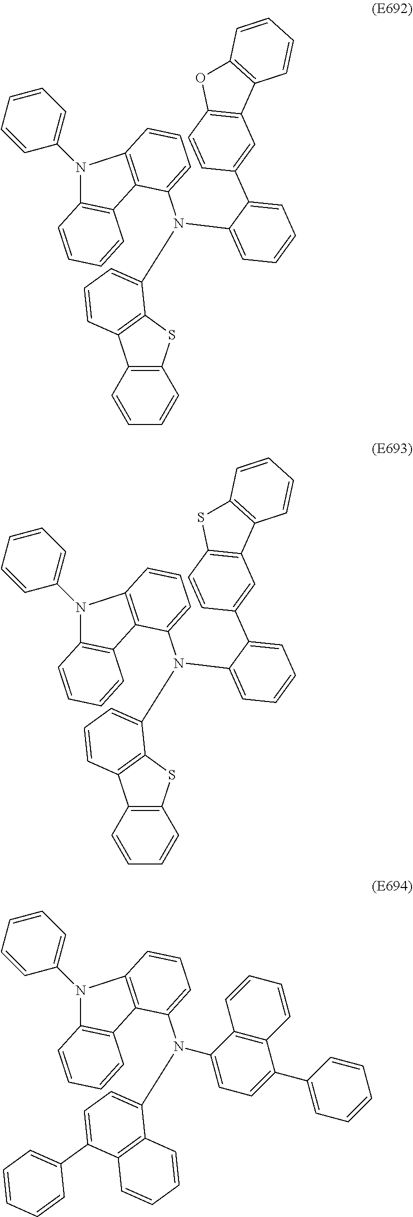 [SMILES: c1ccc(-n2c3ccccc3c3c(N(c4ccccc4-c4ccc5oc6ccccc6c5c4)c4cccc5c4sc4ccccc45)cccc32)cc1 ; c1ccc(-n2c3ccccc3c3c(N(c4ccccc4-c4ccc5sc6ccccc6c5c4)c4cccc5c4sc4ccccc45)cccc32)cc1 ; c1ccc(-c2ccc(N(c3ccc(-c4ccccc4)c4ccccc34)c3cccc4c3c3ccccc3n4-c3ccccc3)c3ccccc23)cc1]

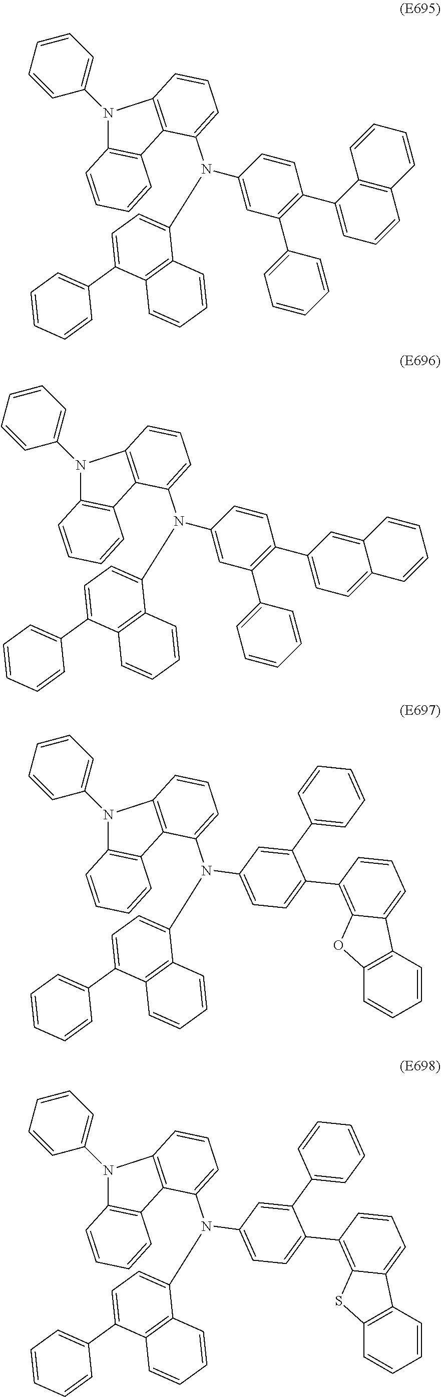 [SMILES: c1ccc(-c2cc(N(c3ccc(-c4ccccc4)c4ccccc34)c3cccc4c3c3ccccc3n4-c3ccccc3)ccc2-c2cccc3ccccc23)cc1 ; c1ccc(-c2cc(N(c3ccc(-c4ccccc4)c4ccccc34)c3cccc4c3c3ccccc3n4-c3ccccc3)ccc2-c2ccc3ccccc3c2)cc1 ; c1ccc(-c2cc(N(c3ccc(-c4ccccc4)c4ccccc34)c3cccc4c3c3ccccc3n4-c3ccccc3)ccc2-c2cccc3c2oc2ccccc23)cc1 ; c1ccc(-c2cc(N(c3ccc(-c4ccccc4)c4ccccc34)c3cccc4c3c3ccccc3n4-c3ccccc3)ccc2-c2cccc3c2sc2ccccc23)cc1]

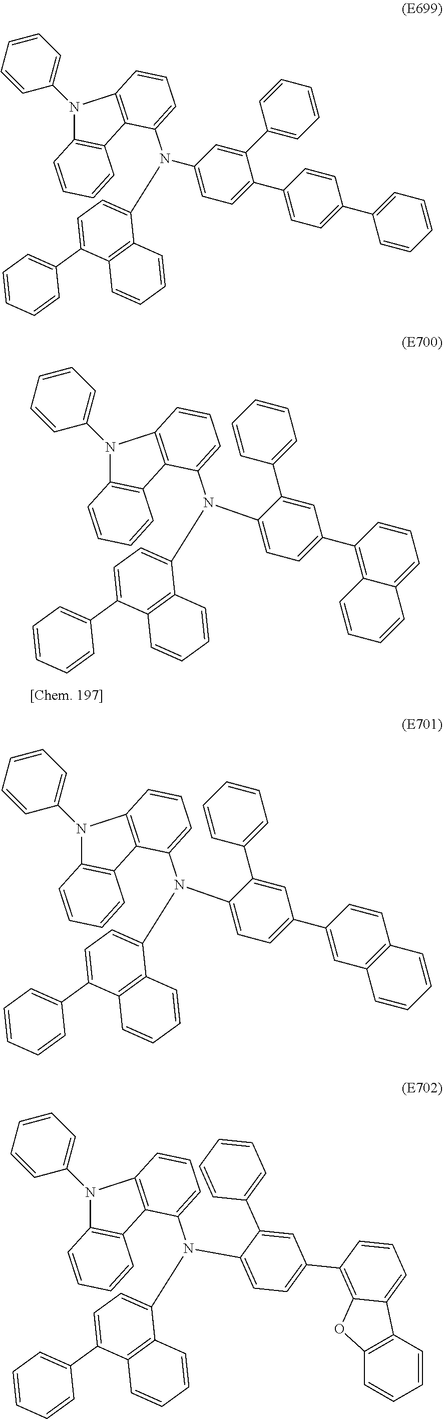 [SMILES: c1ccc(-c2ccc(-c3ccc(N(c4ccc(-c5ccccc5)c5ccccc45)c4cccc5c4c4ccccc4n5-c4ccccc4)cc3-c3ccccc3)cc2)cc1 ; c1ccc(-c2cc(-c3cccc4ccccc34)ccc2N(c2ccc(-c3ccccc3)c3ccccc23)c2cccc3c2c2ccccc2n3-c2ccccc2)cc1 ; c1ccc(-c2cc(-c3ccc4ccccc4c3)ccc2N(c2ccc(-c3ccccc3)c3ccccc23)c2cccc3c2c2ccccc2n3-c2ccccc2)cc1 ; c1ccc(-c2cc(-c3cccc4c3oc3ccccc34)ccc2N(c2ccc(-c3ccccc3)c3ccccc23)c2cccc3c2c2ccccc2n3-c2ccccc2)cc1]

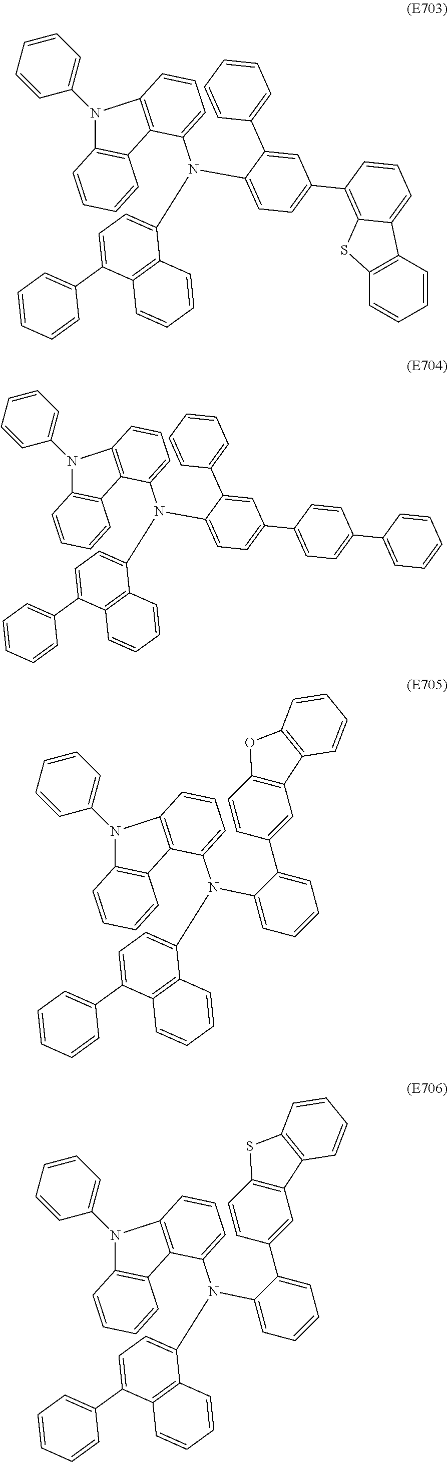 [SMILES: c1ccc(-c2cc(-c3cccc4c3sc3ccccc34)ccc2N(c2ccc(-c3ccccc3)c3ccccc23)c2cccc3c2c2ccccc2n3-c2ccccc2)cc1 ; c1ccc(-c2ccc(-c3ccc(N(c4ccc(-c5ccccc5)c5ccccc45)c4cccc5c4c4ccccc4n5-c4ccccc4)c(-c4ccccc4)c3)cc2)cc1 ; c1ccc(-c2ccc(N(c3ccccc3-c3ccc4oc5ccccc5c4c3)c3cccc4c3c3ccccc3n4-c3ccccc3)c3ccccc23)cc1 ; c1ccc(-c2ccc(N(c3ccccc3-c3ccc4sc5ccccc5c4c3)c3cccc4c3c3ccccc3n4-c3ccccc3)c3ccccc23)cc1]

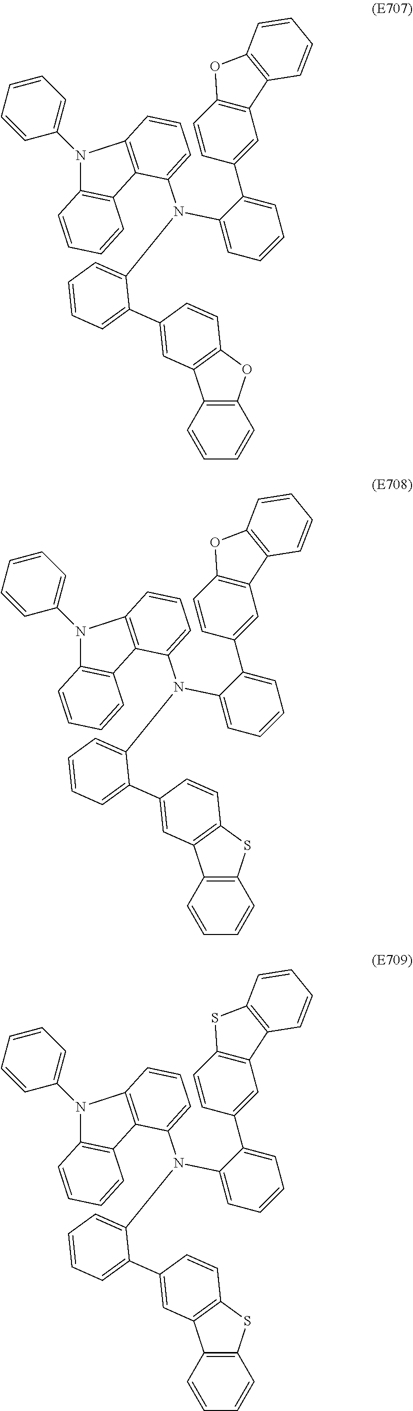

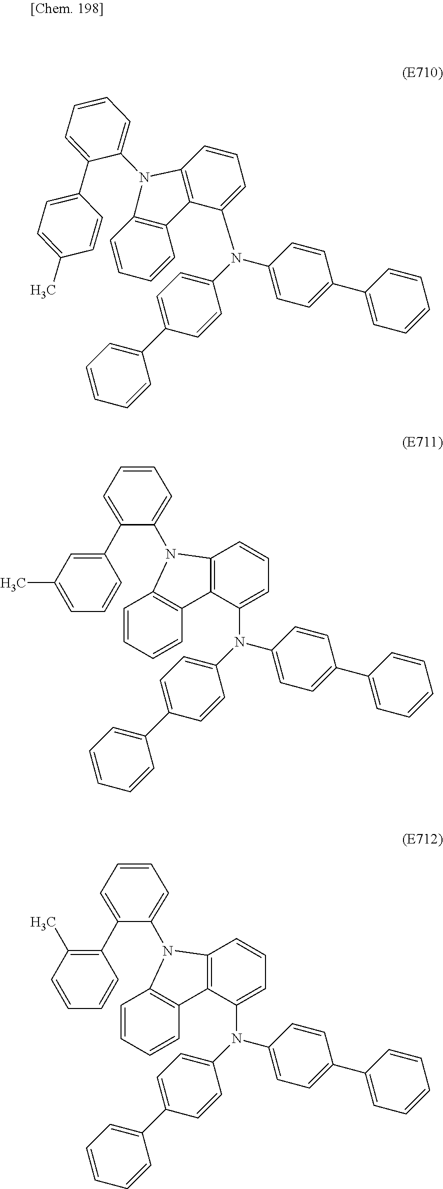 [SMILES: Cc1ccc(-c2ccccc2-n2c3ccccc3c3c(N(c4ccc(-c5ccccc5)cc4)c4ccc(-c5ccccc5)cc4)cccc32)cc1 ; Cc1cccc(-c2ccccc2-n2c3ccccc3c3c(N(c4ccc(-c5ccccc5)cc4)c4ccc(-c5ccccc5)cc4)cccc32)c1 ; Cc1ccccc1-c1ccccc1-n1c2ccccc2c2c(N(c3ccc(-c4ccccc4)cc3)c3ccc(-c4ccccc4)cc3)cccc21]

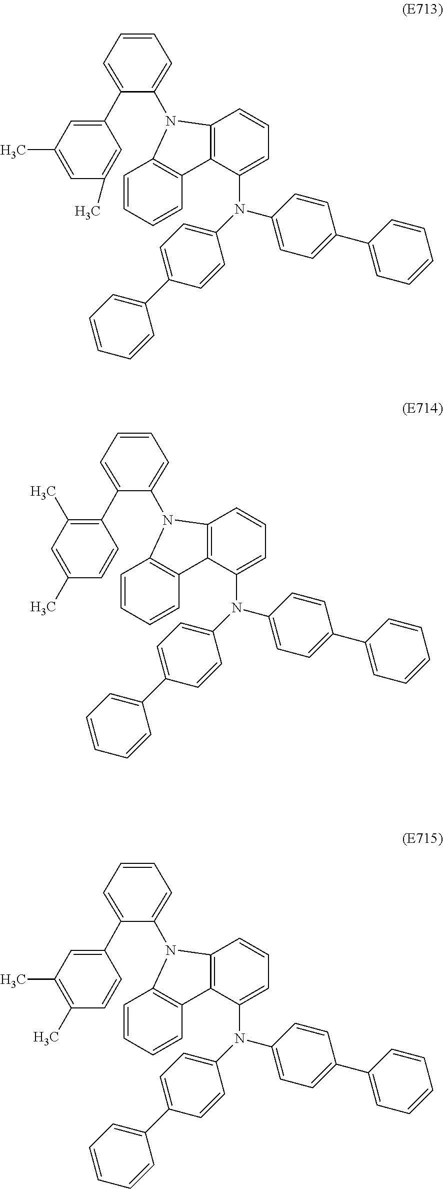 [SMILES: Cc1cc(C)cc(-c2ccccc2-n2c3ccccc3c3c(N(c4ccc(-c5ccccc5)cc4)c4ccc(-c5ccccc5)cc4)cccc32)c1 ; Cc1ccc(-c2ccccc2-n2c3ccccc3c3c(N(c4ccc(-c5ccccc5)cc4)c4ccc(-c5ccccc5)cc4)cccc32)c(C)c1 ; Cc1ccc(-c2ccccc2-n2c3ccccc3c3c(N(c4ccc(-c5ccccc5)cc4)c4ccc(-c5ccccc5)cc4)cccc32)cc1C]

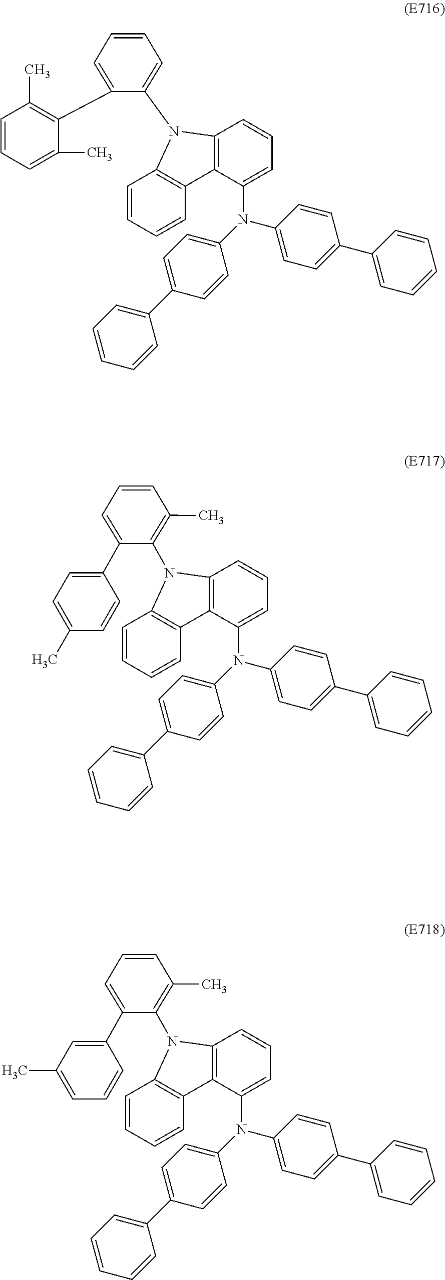 [SMILES: Cc1cccc(C)c1-c1ccccc1-n1c2ccccc2c2c(N(c3ccc(-c4ccccc4)cc3)c3ccc(-c4ccccc4)cc3)cccc21 ; Cc1ccc(-c2cccc(C)c2-n2c3ccccc3c3c(N(c4ccc(-c5ccccc5)cc4)c4ccc(-c5ccccc5)cc4)cccc32)cc1 ; Cc1cccc(-c2cccc(C)c2-n2c3ccccc3c3c(N(c4ccc(-c5ccccc5)cc4)c4ccc(-c5ccccc5)cc4)cccc32)c1]

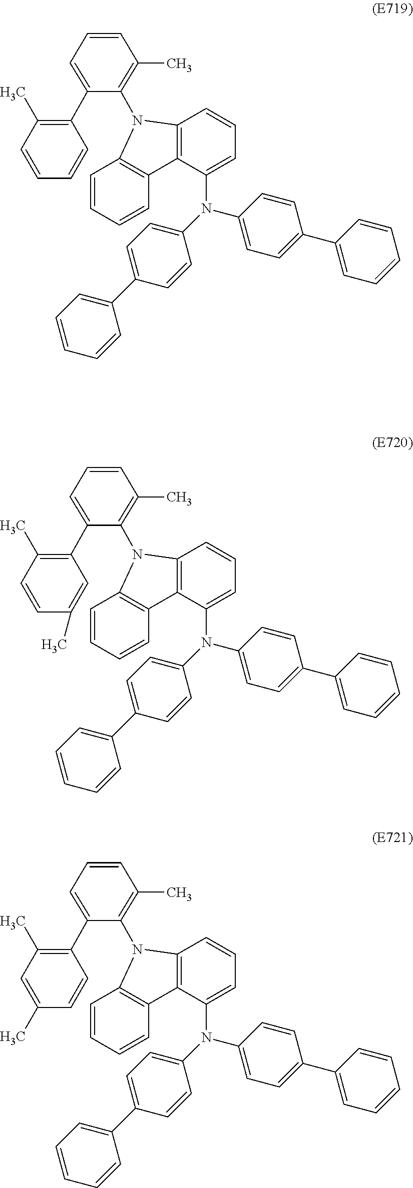 [SMILES: Cc1ccccc1-c1cccc(C)c1-n1c2ccccc2c2c(N(c3ccc(-c4ccccc4)cc3)c3ccc(-c4ccccc4)cc3)cccc21 ; Cc1ccc(C)c(-c2cccc(C)c2-n2c3ccccc3c3c(N(c4ccc(-c5ccccc5)cc4)c4ccc(-c5ccccc5)cc4)cccc32)c1 ; Cc1ccc(-c2cccc(C)c2-n2c3ccccc3c3c(N(c4ccc(-c5ccccc5)cc4)c4ccc(-c5ccccc5)cc4)cccc32)c(C)c1]

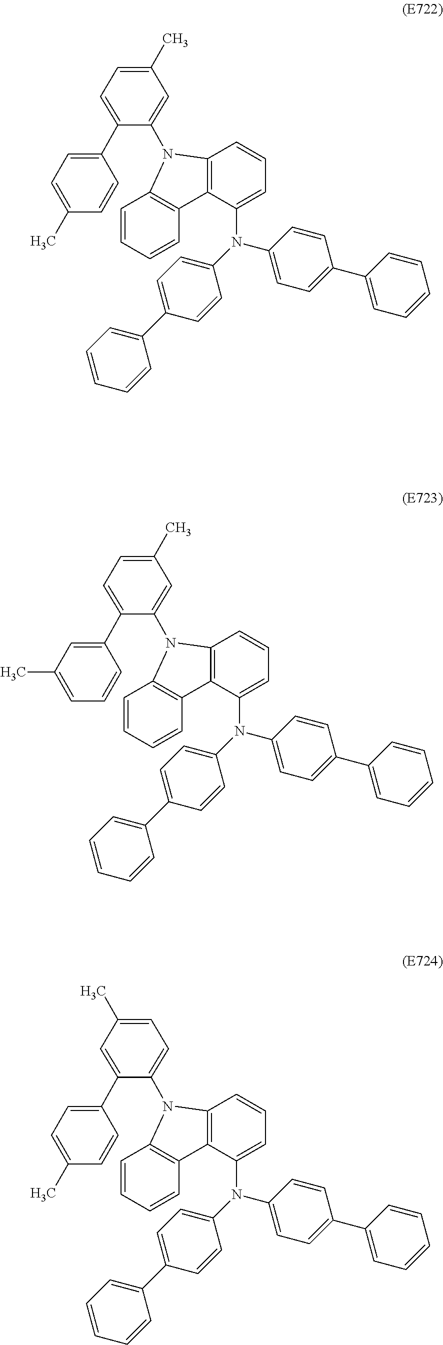 [SMILES: Cc1ccc(-c2ccc(C)cc2-n2c3ccccc3c3c(N(c4ccc(-c5ccccc5)cc4)c4ccc(-c5ccccc5)cc4)cccc32)cc1 ; Cc1cccc(-c2ccc(C)cc2-n2c3ccccc3c3c(N(c4ccc(-c5ccccc5)cc4)c4ccc(-c5ccccc5)cc4)cccc32)c1 ; Cc1ccc(-c2cc(C)ccc2-n2c3ccccc3c3c(N(c4ccc(-c5ccccc5)cc4)c4ccc(-c5ccccc5)cc4)cccc32)cc1]

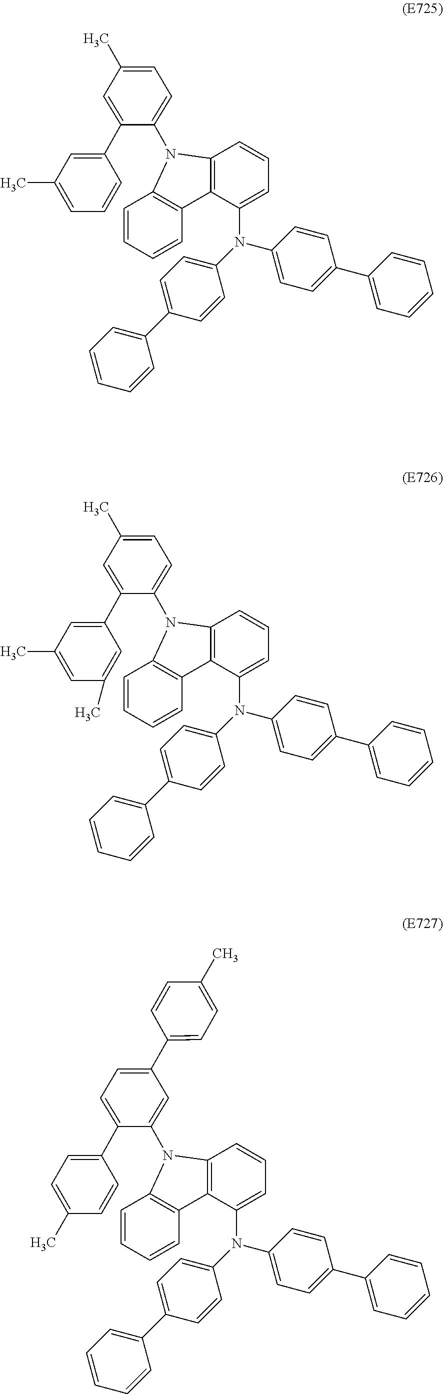 [SMILES: Cc1cccc(-c2cc(C)ccc2-n2c3ccccc3c3c(N(c4ccc(-c5ccccc5)cc4)c4ccc(-c5ccccc5)cc4)cccc32)c1 ; Cc1cc(C)cc(-c2cc(C)ccc2-n2c3ccccc3c3c(N(c4ccc(-c5ccccc5)cc4)c4ccc(-c5ccccc5)cc4)cccc32)c1 ; Cc1ccc(-c2ccc(-c3ccc(C)cc3)c(-n3c4ccccc4c4c(N(c5ccc(-c6ccccc6)cc5)c5ccc(-c6ccccc6)cc5)cccc43)c2)cc1]

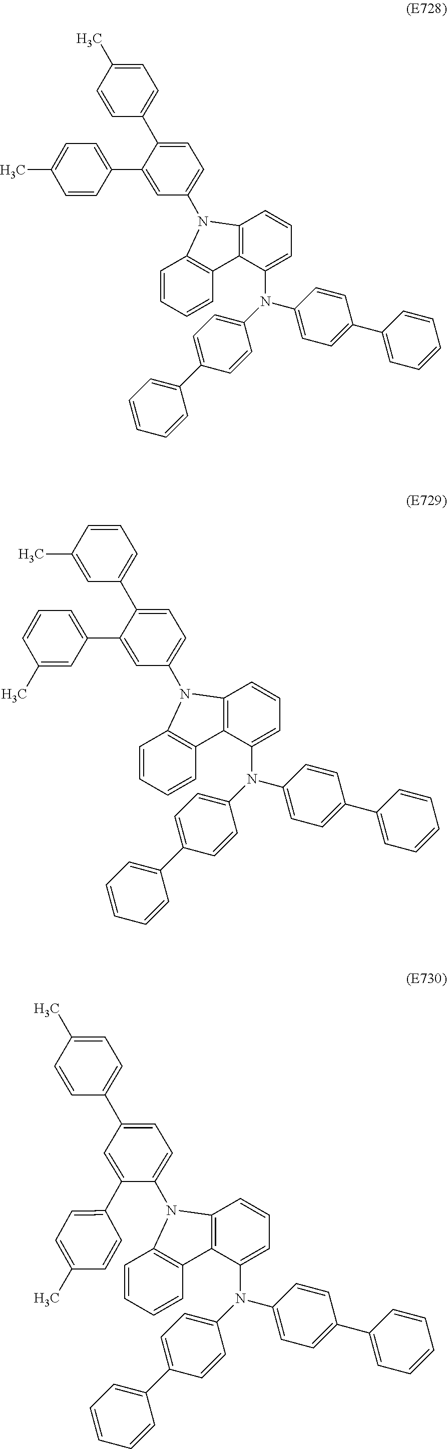 [SMILES: Cc1ccc(-c2ccc(-n3c4ccccc4c4c(N(c5ccc(-c6ccccc6)cc5)c5ccc(-c6ccccc6)cc5)cccc43)cc2-c2ccc(C)cc2)cc1 ; Cc1cccc(-c2ccc(-n3c4ccccc4c4c(N(c5ccc(-c6ccccc6)cc5)c5ccc(-c6ccccc6)cc5)cccc43)cc2-c2cccc(C)c2)c1 ; Cc1ccc(-c2ccc(-n3c4ccccc4c4c(N(c5ccc(-c6ccccc6)cc5)c5ccc(-c6ccccc6)cc5)cccc43)c(-c3ccc(C)cc3)c2)cc1]

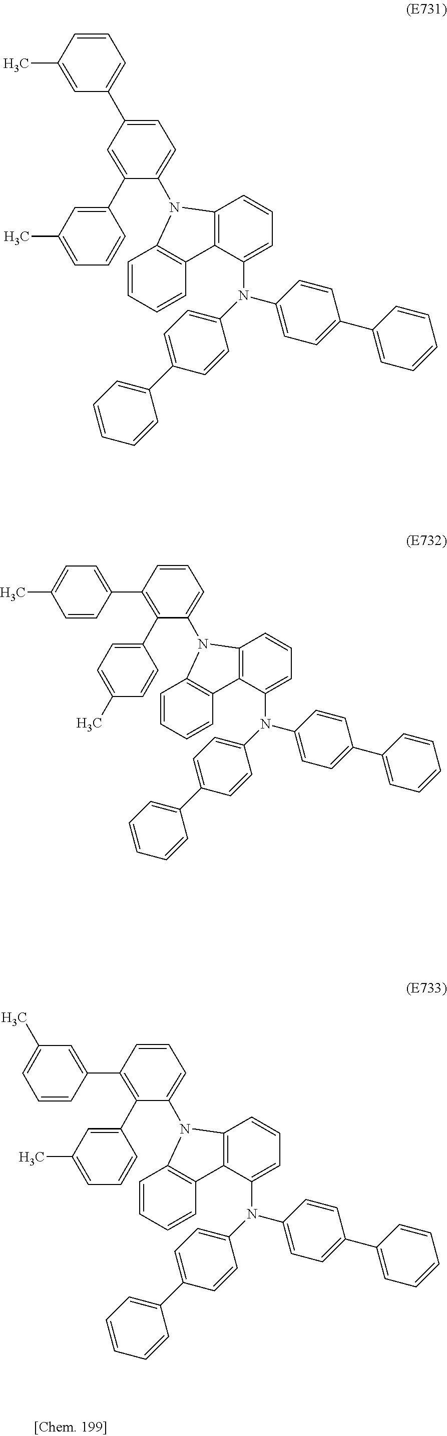 [SMILES: Cc1cccc(-c2ccc(-n3c4ccccc4c4c(N(c5ccc(-c6ccccc6)cc5)c5ccc(-c6ccccc6)cc5)cccc43)c(-c3cccc(C)c3)c2)c1 ; Cc1ccc(-c2cccc(-n3c4ccccc4c4c(N(c5ccc(-c6ccccc6)cc5)c5ccc(-c6ccccc6)cc5)cccc43)c2-c2ccc(C)cc2)cc1 ; Cc1cccc(-c2cccc(-n3c4ccccc4c4c(N(c5ccc(-c6ccccc6)cc5)c5ccc(-c6ccccc6)cc5)cccc43)c2-c2cccc(C)c2)c1]

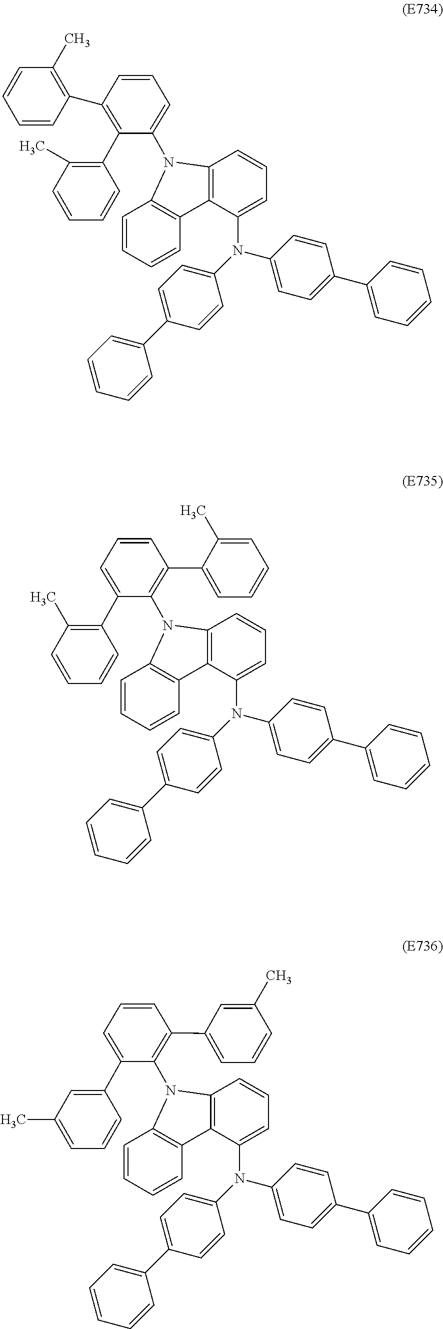 [SMILES: Cc1ccccc1-c1cccc(-n2c3ccccc3c3c(N(c4ccc(-c5ccccc5)cc4)c4ccc(-c5ccccc5)cc4)cccc32)c1-c1ccccc1C ; Cc1ccccc1-c1cccc(-c2ccccc2C)c1-n1c2ccccc2c2c(N(c3ccc(-c4ccccc4)cc3)c3ccc(-c4ccccc4)cc3)cccc21 ; Cc1cccc(-c2cccc(-c3cccc(C)c3)c2-n2c3ccccc3c3c(N(c4ccc(-c5ccccc5)cc4)c4ccc(-c5ccccc5)cc4)cccc32)c1]

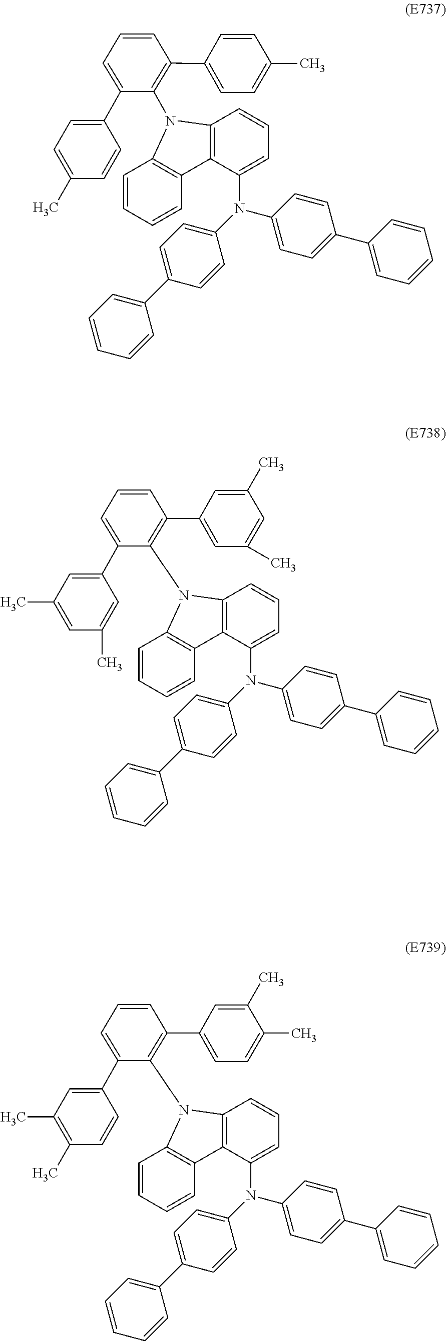 [SMILES: Cc1ccc(-c2cccc(-c3ccc(C)cc3)c2-n2c3ccccc3c3c(N(c4ccc(-c5ccccc5)cc4)c4ccc(-c5ccccc5)cc4)cccc32)cc1 ; Cc1cc(C)cc(-c2cccc(-c3cc(C)cc(C)c3)c2-n2c3ccccc3c3c(N(c4ccc(-c5ccccc5)cc4)c4ccc(-c5ccccc5)cc4)cccc32)c1 ; Cc1ccc(-c2cccc(-c3ccc(C)c(C)c3)c2-n2c3ccccc3c3c(N(c4ccc(-c5ccccc5)cc4)c4ccc(-c5ccccc5)cc4)cccc32)cc1C]

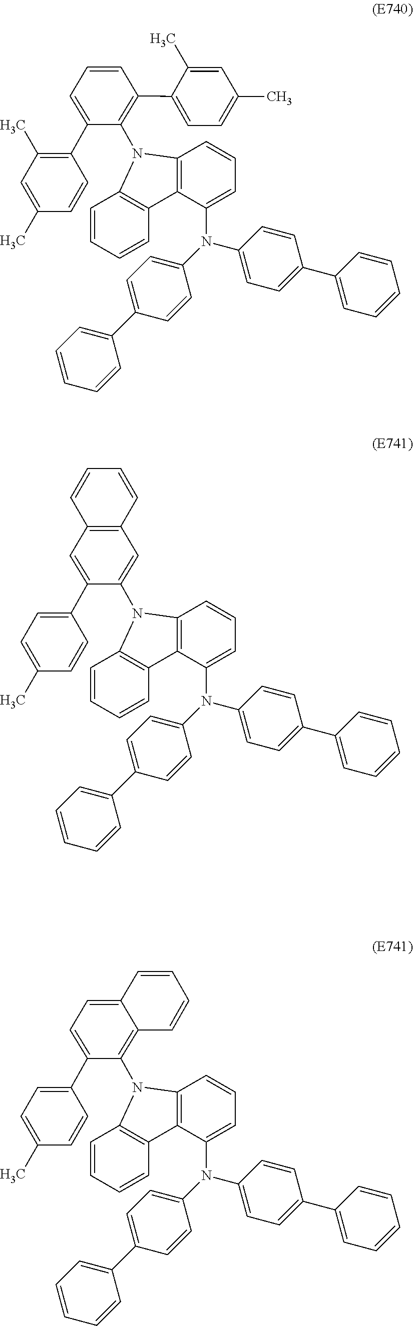 [SMILES: Cc1ccc(-c2cccc(-c3ccc(C)cc3C)c2-n2c3ccccc3c3c(N(c4ccc(-c5ccccc5)cc4)c4ccc(-c5ccccc5)cc4)cccc32)c(C)c1 ; Cc1ccc(-c2cc3ccccc3cc2-n2c3ccccc3c3c(N(c4ccc(-c5ccccc5)cc4)c4ccc(-c5ccccc5)cc4)cccc32)cc1 ; Cc1ccc(-c2ccc3ccccc3c2-n2c3ccccc3c3c(N(c4ccc(-c5ccccc5)cc4)c4ccc(-c5ccccc5)cc4)cccc32)cc1]

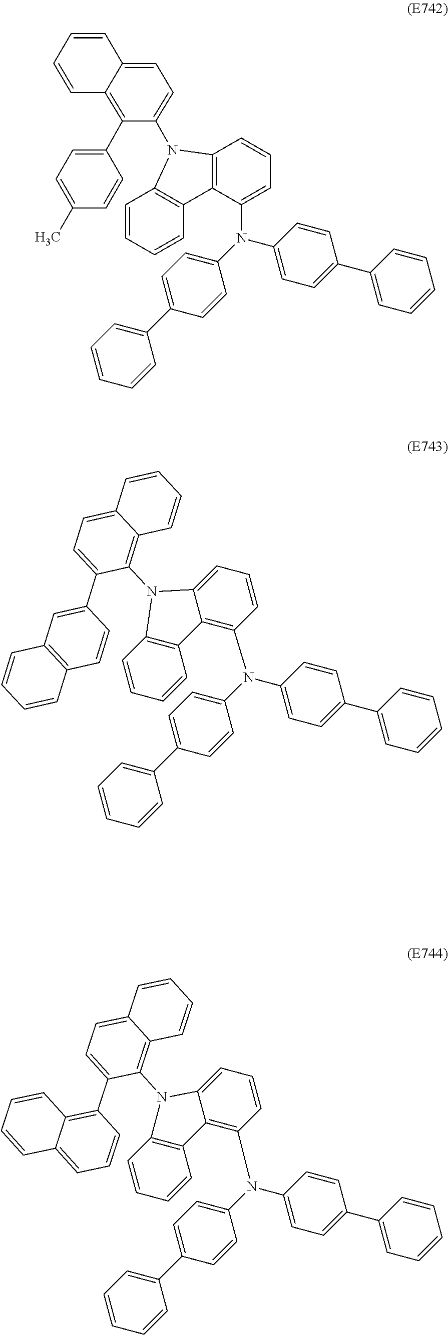 [SMILES: Cc1ccc(-c2c(-n3c4ccccc4c4c(N(c5ccc(-c6ccccc6)cc5)c5ccc(-c6ccccc6)cc5)cccc43)ccc3ccccc23)cc1 ; c1ccc(-c2ccc(N(c3ccc(-c4ccccc4)cc3)c3cccc4c3c3ccccc3n4-c3c(-c4ccc5ccccc5c4)ccc4ccccc34)cc2)cc1 ; c1ccc(-c2ccc(N(c3ccc(-c4ccccc4)cc3)c3cccc4c3c3ccccc3n4-c3c(-c4cccc5ccccc45)ccc4ccccc34)cc2)cc1]

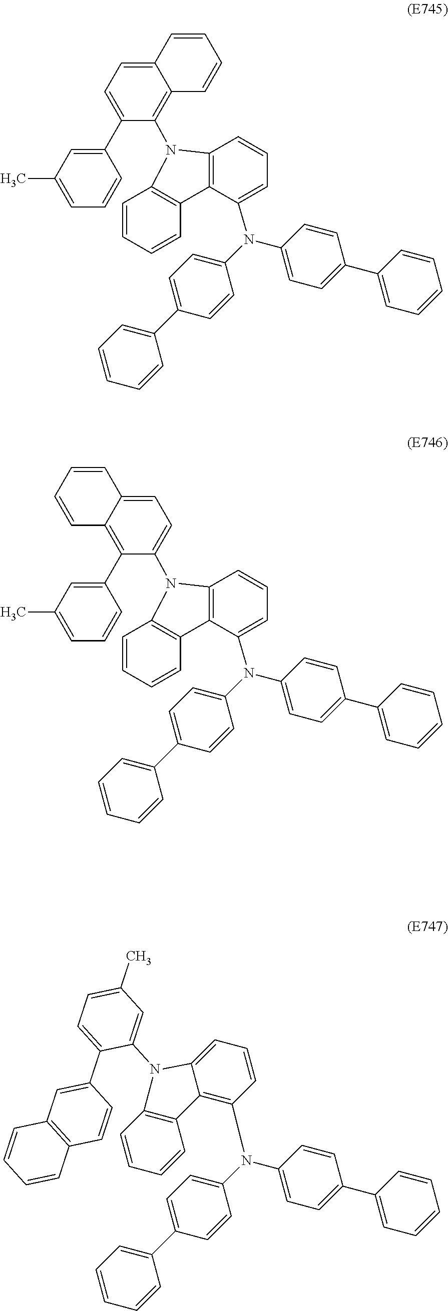 [SMILES: Cc1cccc(-c2ccc3ccccc3c2-n2c3ccccc3c3c(N(c4ccc(-c5ccccc5)cc4)c4ccc(-c5ccccc5)cc4)cccc32)c1 ; Cc1cccc(-c2c(-n3c4ccccc4c4c(N(c5ccc(-c6ccccc6)cc5)c5ccc(-c6ccccc6)cc5)cccc43)ccc3ccccc23)c1 ; Cc1ccc(-c2ccc3ccccc3c2)c(-n2c3ccccc3c3c(N(c4ccc(-c5ccccc5)cc4)c4ccc(-c5ccccc5)cc4)cccc32)c1]

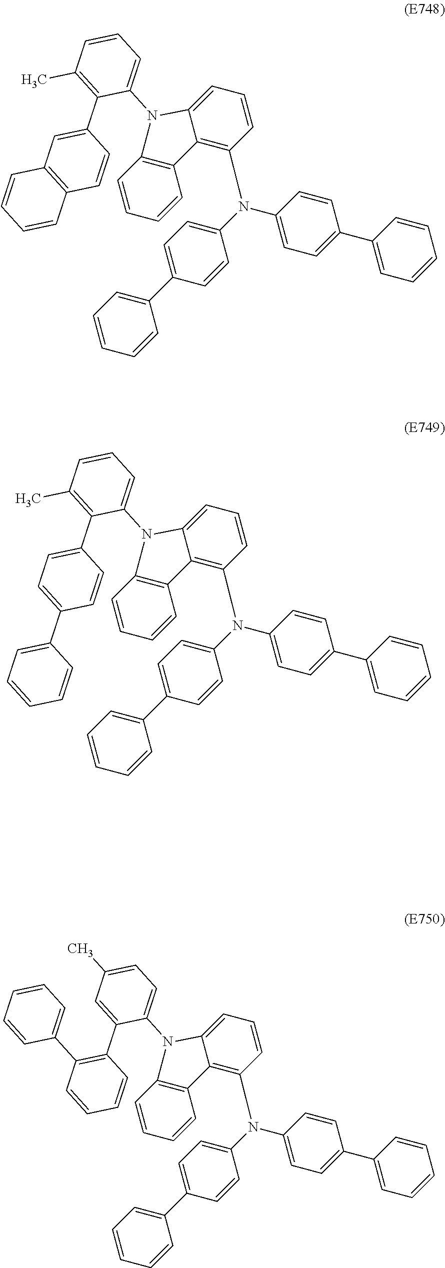 [SMILES: Cc1cccc(-n2c3ccccc3c3c(N(c4ccc(-c5ccccc5)cc4)c4ccc(-c5ccccc5)cc4)cccc32)c1-c1ccc2ccccc2c1 ; Cc1cccc(-n2c3ccccc3c3c(N(c4ccc(-c5ccccc5)cc4)c4ccc(-c5ccccc5)cc4)cccc32)c1-c1ccc(-c2ccccc2)cc1 ; Cc1ccc(-n2c3ccccc3c3c(N(c4ccc(-c5ccccc5)cc4)c4ccc(-c5ccccc5)cc4)cccc32)c(-c2ccccc2-c2ccccc2)c1]

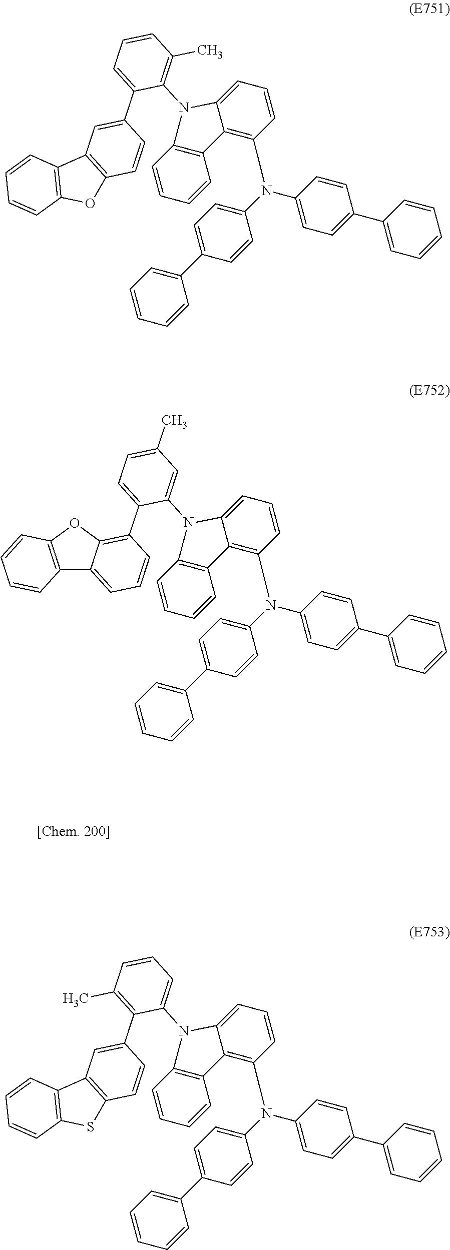 [SMILES: Cc1cccc(-c2ccc3oc4ccccc4c3c2)c1-n1c2ccccc2c2c(N(c3ccc(-c4ccccc4)cc3)c3ccc(-c4ccccc4)cc3)cccc21 ; Cc1ccc(-c2cccc3c2oc2ccccc23)c(-n2c3ccccc3c3c(N(c4ccc(-c5ccccc5)cc4)c4ccc(-c5ccccc5)cc4)cccc32)c1 ; Cc1cccc(-n2c3ccccc3c3c(N(c4ccc(-c5ccccc5)cc4)c4ccc(-c5ccccc5)cc4)cccc32)c1-c1ccc2sc3ccccc3c2c1]

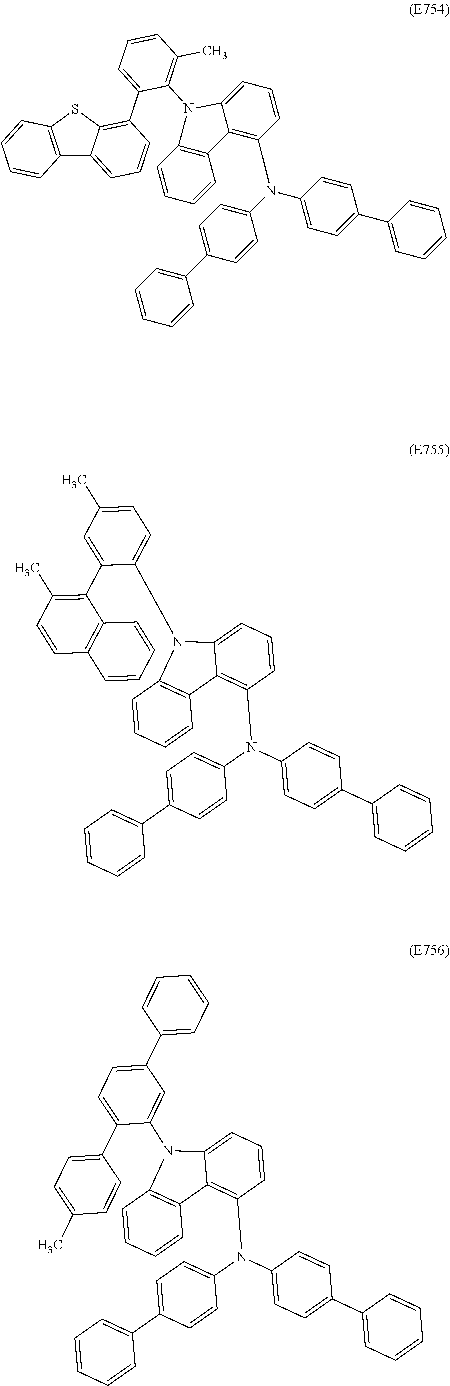 [SMILES: Cc1cccc(-c2cccc3c2sc2ccccc23)c1-n1c2ccccc2c2c(N(c3ccc(-c4ccccc4)cc3)c3ccc(-c4ccccc4)cc3)cccc21 ; Cc1ccc(-n2c3ccccc3c3c(N(c4ccc(-c5ccccc5)cc4)c4ccc(-c5ccccc5)cc4)cccc32)c(-c2c(C)ccc3ccccc23)c1 ; Cc1ccc(-c2ccc(-c3ccccc3)cc2-n2c3ccccc3c3c(N(c4ccc(-c5ccccc5)cc4)c4ccc(-c5ccccc5)cc4)cccc32)cc1]

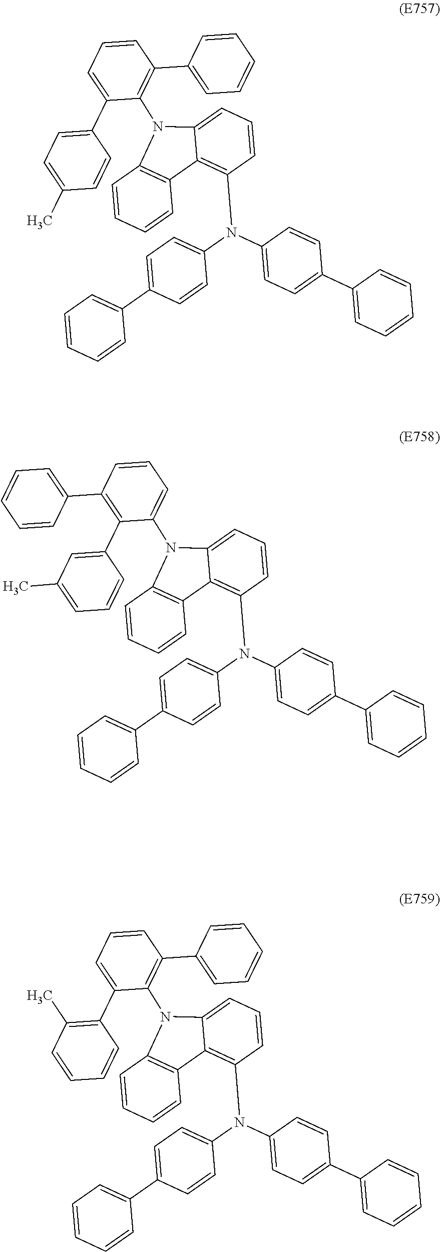 [SMILES: Cc1ccc(-c2cccc(-c3ccccc3)c2-n2c3ccccc3c3c(N(c4ccc(-c5ccccc5)cc4)c4ccc(-c5ccccc5)cc4)cccc32)cc1 ; Cc1cccc(-c2c(-c3ccccc3)cccc2-n2c3ccccc3c3c(N(c4ccc(-c5ccccc5)cc4)c4ccc(-c5ccccc5)cc4)cccc32)c1 ; Cc1ccccc1-c1cccc(-c2ccccc2)c1-n1c2ccccc2c2c(N(c3ccc(-c4ccccc4)cc3)c3ccc(-c4ccccc4)cc3)cccc21]

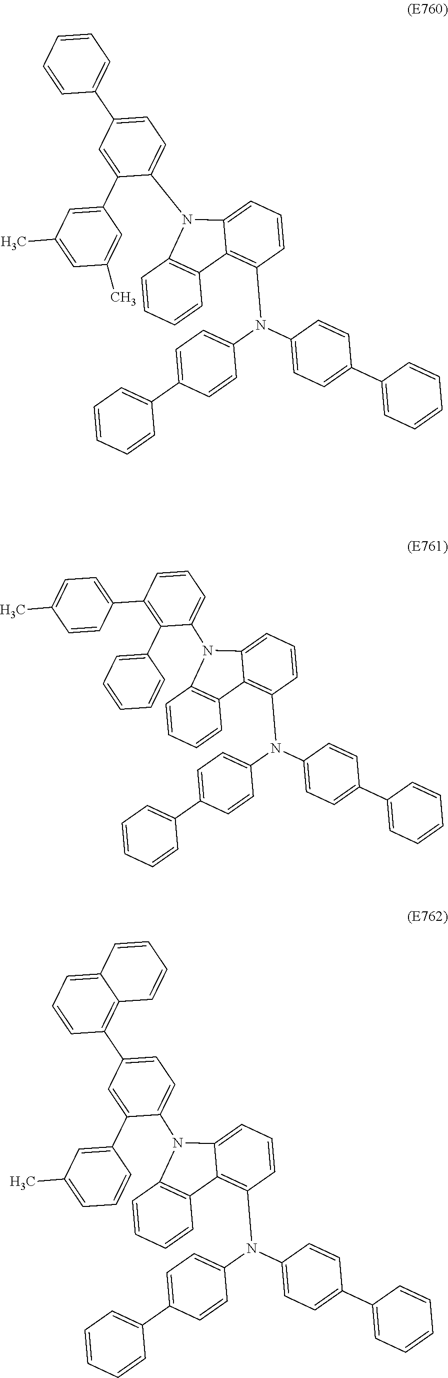 [SMILES: Cc1cc(C)cc(-c2cc(-c3ccccc3)ccc2-n2c3ccccc3c3c(N(c4ccc(-c5ccccc5)cc4)c4ccc(-c5ccccc5)cc4)cccc32)c1 ; Cc1ccc(-c2cccc(-n3c4ccccc4c4c(N(c5ccc(-c6ccccc6)cc5)c5ccc(-c6ccccc6)cc5)cccc43)c2-c2ccccc2)cc1 ; Cc1cccc(-c2cc(-c3cccc4ccccc34)ccc2-n2c3ccccc3c3c(N(c4ccc(-c5ccccc5)cc4)c4ccc(-c5ccccc5)cc4)cccc32)c1]

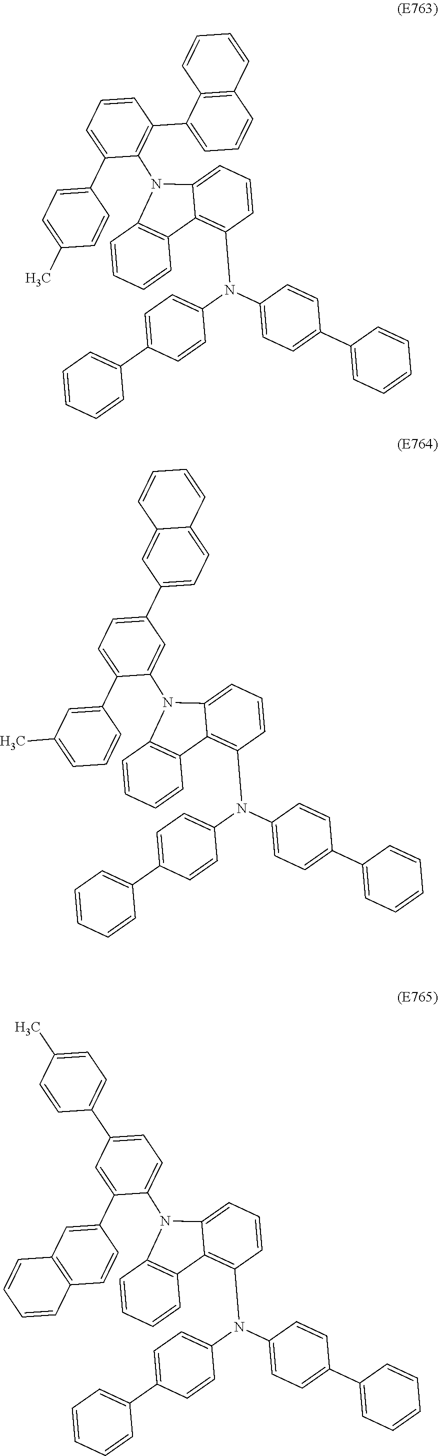 [SMILES: Cc1ccc(-c2cccc(-c3cccc4ccccc34)c2-n2c3ccccc3c3c(N(c4ccc(-c5ccccc5)cc4)c4ccc(-c5ccccc5)cc4)cccc32)cc1 ; Cc1cccc(-c2ccc(-c3ccc4ccccc4c3)cc2-n2c3ccccc3c3c(N(c4ccc(-c5ccccc5)cc4)c4ccc(-c5ccccc5)cc4)cccc32)c1 ; Cc1ccc(-c2ccc(-n3c4ccccc4c4c(N(c5ccc(-c6ccccc6)cc5)c5ccc(-c6ccccc6)cc5)cccc43)c(-c3ccc4ccccc4c3)c2)cc1]

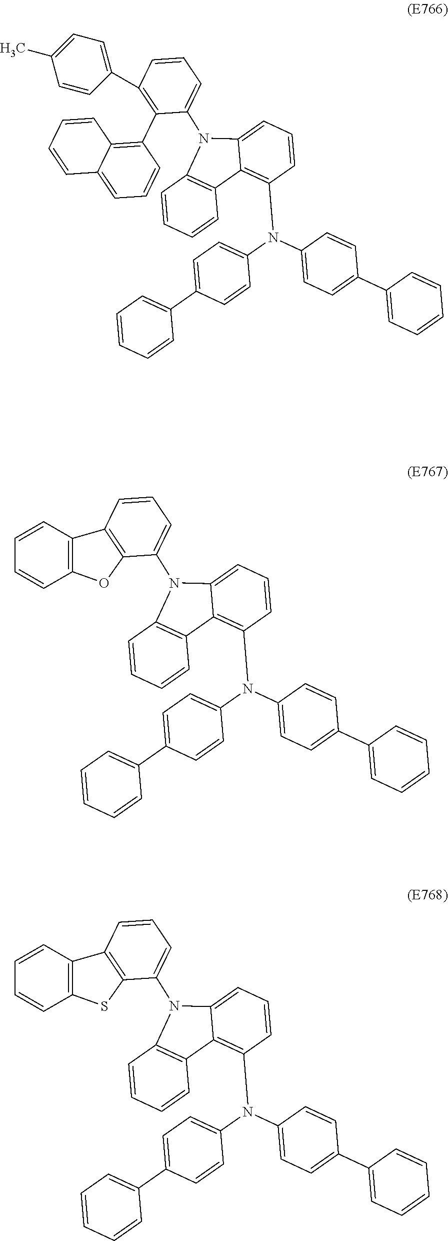 [SMILES: Cc1ccc(-c2cccc(-n3c4ccccc4c4c(N(c5ccc(-c6ccccc6)cc5)c5ccc(-c6ccccc6)cc5)cccc43)c2-c2cccc3ccccc23)cc1 ; c1ccc(-c2ccc(N(c3ccc(-c4ccccc4)cc3)c3cccc4c3c3ccccc3n4-c3cccc4c3oc3ccccc34)cc2)cc1 ; c1ccc(-c2ccc(N(c3ccc(-c4ccccc4)cc3)c3cccc4c3c3ccccc3n4-c3cccc4c3sc3ccccc34)cc2)cc1]

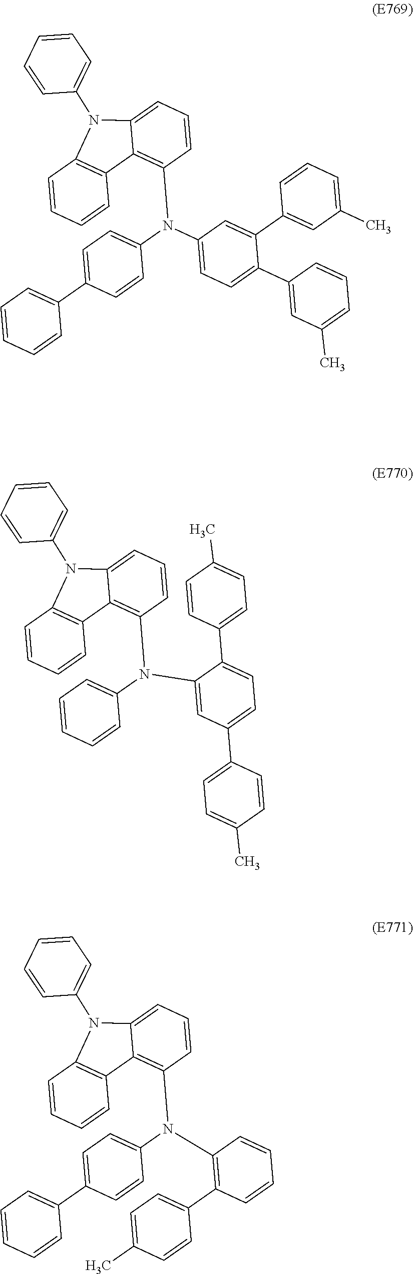 [SMILES: Cc1cccc(-c2ccc(N(c3ccc(-c4ccccc4)cc3)c3cccc4c3c3ccccc3n4-c3ccccc3)cc2-c2cccc(C)c2)c1 ; Cc1ccc(-c2ccc(-c3ccc(C)cc3)c(N(c3ccccc3)c3cccc4c3c3ccccc3n4-c3ccccc3)c2)cc1 ; Cc1ccc(-c2ccccc2N(c2ccc(-c3ccccc3)cc2)c2cccc3c2c2ccccc2n3-c2ccccc2)cc1]

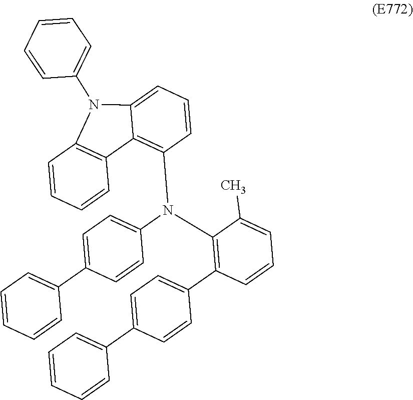 [SMILES: Cc1cccc(-c2ccc(-c3ccccc3)cc2)c1N(c1ccc(-c2ccccc2)cc1)c1cccc2c1c1ccccc1n2-c1ccccc1]

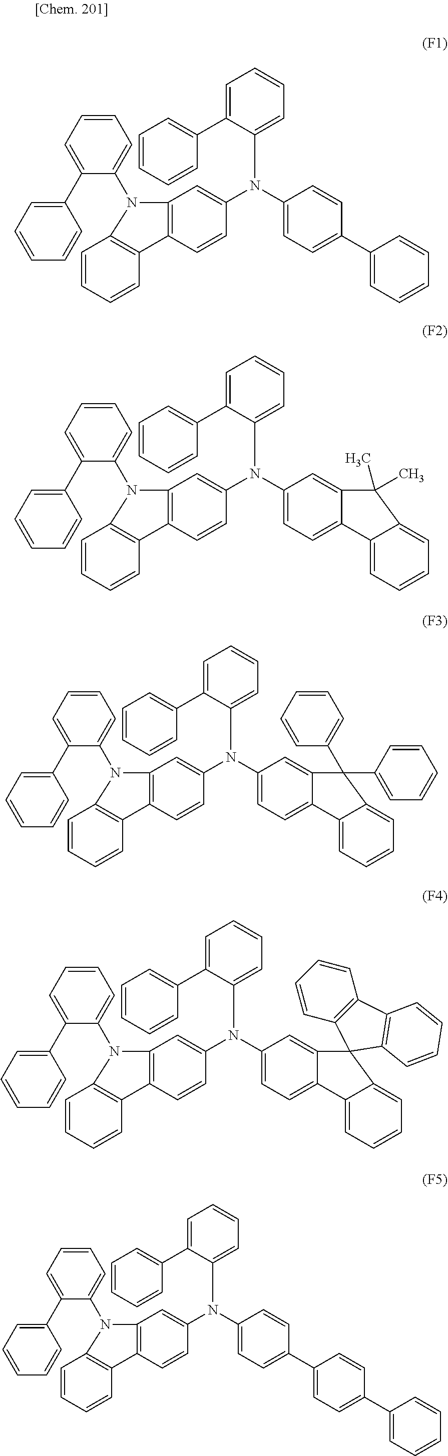 [SMILES: c1ccc(-c2ccc(N(c3ccc4c5ccccc5n(-c5ccccc5-c5ccccc5)c4c3)c3ccccc3-c3ccccc3)cc2)cc1 ; CC1(C)c2ccccc2-c2ccc(N(c3ccc4c5ccccc5n(-c5ccccc5-c5ccccc5)c4c3)c3ccccc3-c3ccccc3)cc21 ; c1ccc(-c2ccccc2N(c2ccc3c(c2)C(c2ccccc2)(c2ccccc2)c2ccccc2-3)c2ccc3c4ccccc4n(-c4ccccc4-c4ccccc4)c3c2)cc1 ; c1ccc(-c2ccccc2N(c2ccc3c(c2)C2(c4ccccc4-c4ccccc42)c2ccccc2-3)c2ccc3c4ccccc4n(-c4ccccc4-c4ccccc4)c3c2)cc1 ; c1ccc(-c2ccc(-c3ccc(N(c4ccc5c6ccccc6n(-c6ccccc6-c6ccccc6)c5c4)c4ccccc4-c4ccccc4)cc3)cc2)cc1]

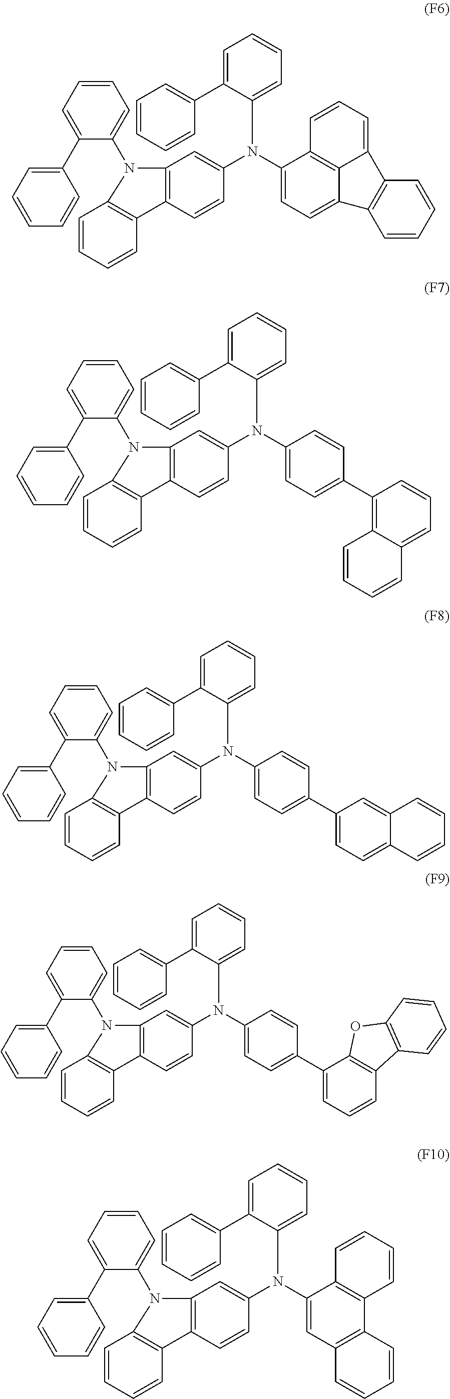 [SMILES: c1ccc(-c2ccccc2N(c2ccc3c4ccccc4n(-c4ccccc4-c4ccccc4)c3c2)c2ccc3c4c(cccc24)-c2ccccc2-3)cc1 ; c1ccc(-c2ccccc2N(c2ccc(-c3cccc4ccccc34)cc2)c2ccc3c4ccccc4n(-c4ccccc4-c4ccccc4)c3c2)cc1 ; c1ccc(-c2ccccc2N(c2ccc(-c3ccc4ccccc4c3)cc2)c2ccc3c4ccccc4n(-c4ccccc4-c4ccccc4)c3c2)cc1 ; c1ccc(-c2ccccc2N(c2ccc(-c3cccc4c3oc3ccccc34)cc2)c2ccc3c4ccccc4n(-c4ccccc4-c4ccccc4)c3c2)cc1 ; c1ccc(-c2ccccc2N(c2ccc3c4ccccc4n(-c4ccccc4-c4ccccc4)c3c2)c2cc3ccccc3c3ccccc23)cc1]

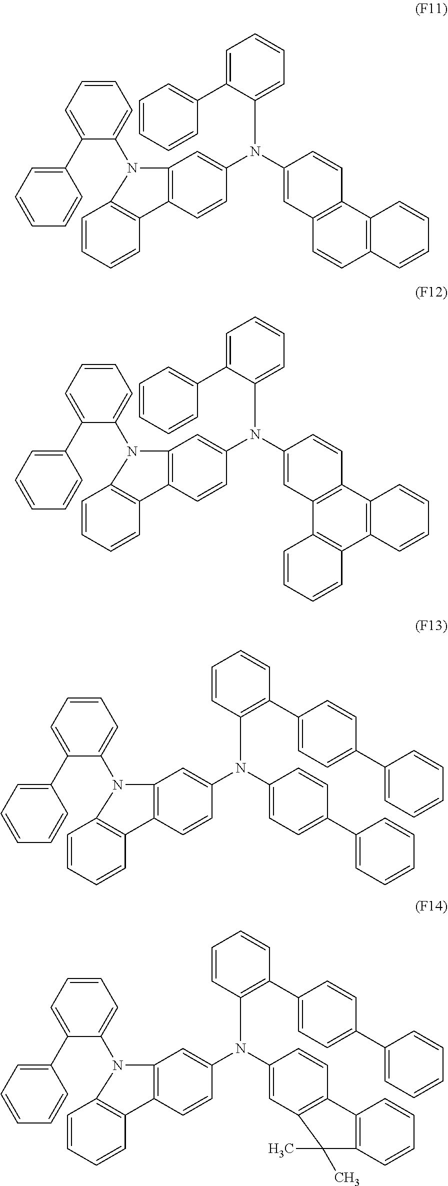 [SMILES: c1ccc(-c2ccccc2N(c2ccc3c(ccc4ccccc43)c2)c2ccc3c4ccccc4n(-c4ccccc4-c4ccccc4)c3c2)cc1 ; c1ccc(-c2ccccc2N(c2ccc3c4ccccc4c4ccccc4c3c2)c2ccc3c4ccccc4n(-c4ccccc4-c4ccccc4)c3c2)cc1 ; c1ccc(-c2ccc(-c3ccccc3N(c3ccc(-c4ccccc4)cc3)c3ccc4c5ccccc5n(-c5ccccc5-c5ccccc5)c4c3)cc2)cc1 ; CC1(C)c2ccccc2-c2ccc(N(c3ccc4c5ccccc5n(-c5ccccc5-c5ccccc5)c4c3)c3ccccc3-c3ccc(-c4ccccc4)cc3)cc21]

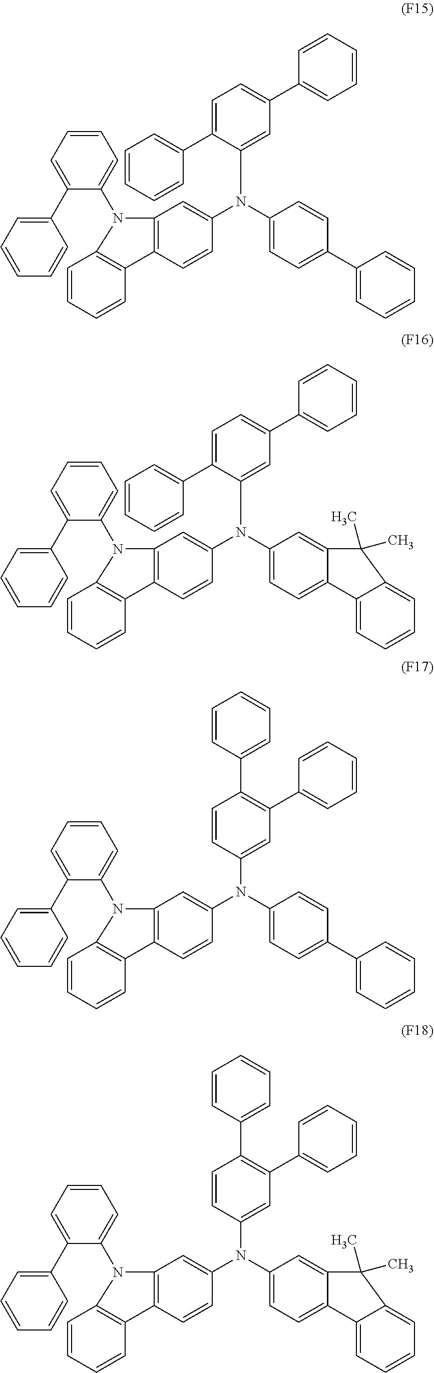 [SMILES: c1ccc(-c2ccc(N(c3ccc4c5ccccc5n(-c5ccccc5-c5ccccc5)c4c3)c3cc(-c4ccccc4)ccc3-c3ccccc3)cc2)cc1 ; CC1(C)c2ccccc2-c2ccc(N(c3ccc4c5ccccc5n(-c5ccccc5-c5ccccc5)c4c3)c3cc(-c4ccccc4)ccc3-c3ccccc3)cc21 ; c1ccc(-c2ccc(N(c3ccc(-c4ccccc4)c(-c4ccccc4)c3)c3ccc4c5ccccc5n(-c5ccccc5-c5ccccc5)c4c3)cc2)cc1 ; CC1(C)c2ccccc2-c2ccc(N(c3ccc(-c4ccccc4)c(-c4ccccc4)c3)c3ccc4c5ccccc5n(-c5ccccc5-c5ccccc5)c4c3)cc21]

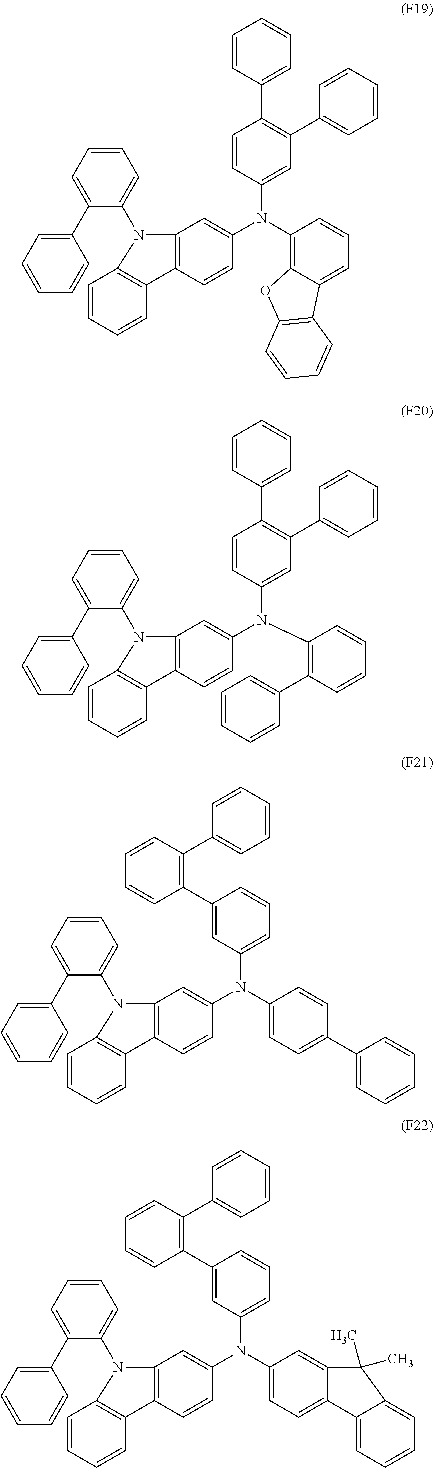

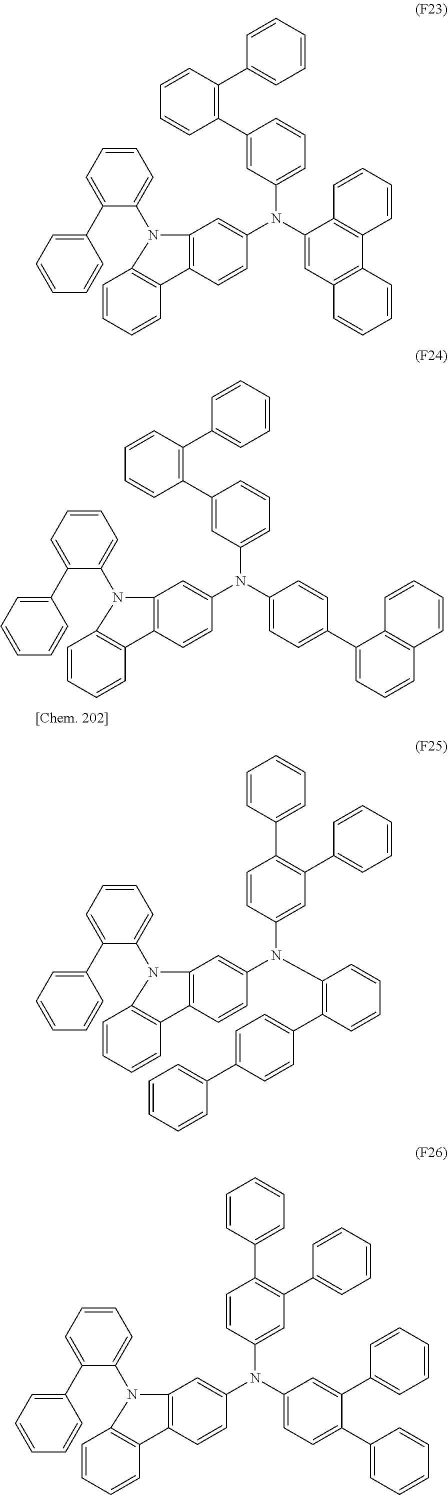 [SMILES: c1ccc(-c2ccccc2-c2cccc(N(c3ccc4c5ccccc5n(-c5ccccc5-c5ccccc5)c4c3)c3cc4ccccc4c4ccccc34)c2)cc1 ; c1ccc(-c2ccccc2-c2cccc(N(c3ccc(-c4cccc5ccccc45)cc3)c3ccc4c5ccccc5n(-c5ccccc5-c5ccccc5)c4c3)c2)cc1 ; c1ccc(-c2ccc(-c3ccccc3N(c3ccc(-c4ccccc4)c(-c4ccccc4)c3)c3ccc4c5ccccc5n(-c5ccccc5-c5ccccc5)c4c3)cc2)cc1 ; c1ccc(-c2ccc(N(c3ccc(-c4ccccc4)c(-c4ccccc4)c3)c3ccc4c5ccccc5n(-c5ccccc5-c5ccccc5)c4c3)cc2-c2ccccc2)cc1]

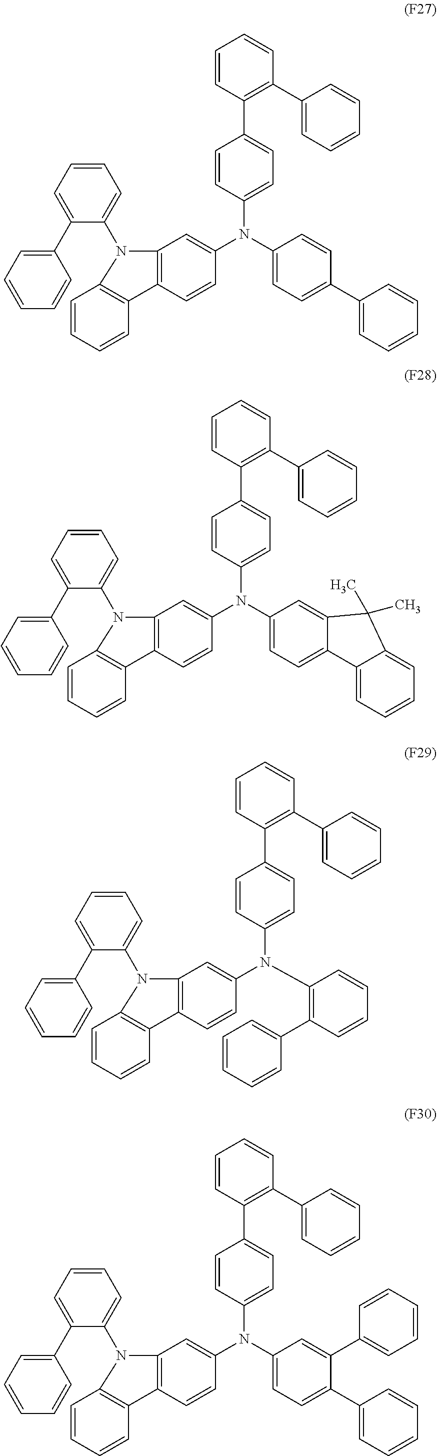 [SMILES: c1ccc(-c2ccc(N(c3ccc(-c4ccccc4-c4ccccc4)cc3)c3ccc4c5ccccc5n(-c5ccccc5-c5ccccc5)c4c3)cc2)cc1 ; CC1(C)c2ccccc2-c2ccc(N(c3ccc(-c4ccccc4-c4ccccc4)cc3)c3ccc4c5ccccc5n(-c5ccccc5-c5ccccc5)c4c3)cc21 ; c1ccc(-c2ccccc2-c2ccc(N(c3ccc4c5ccccc5n(-c5ccccc5-c5ccccc5)c4c3)c3ccccc3-c3ccccc3)cc2)cc1 ; c1ccc(-c2ccccc2-c2ccc(N(c3ccc(-c4ccccc4)c(-c4ccccc4)c3)c3ccc4c5ccccc5n(-c5ccccc5-c5ccccc5)c4c3)cc2)cc1]

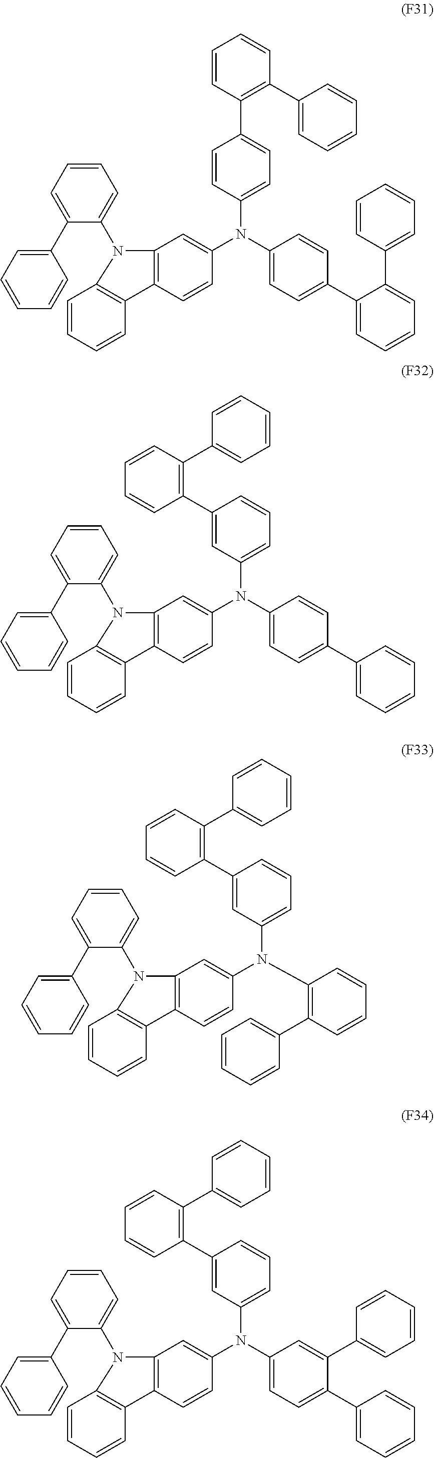 [SMILES: c1ccc(-c2ccccc2-c2ccc(N(c3ccc(-c4ccccc4-c4ccccc4)cc3)c3ccc4c5ccccc5n(-c5ccccc5-c5ccccc5)c4c3)cc2)cc1 ; c1ccc(-c2ccc(N(c3cccc(-c4ccccc4-c4ccccc4)c3)c3ccc4c5ccccc5n(-c5ccccc5-c5ccccc5)c4c3)cc2)cc1 ; c1ccc(-c2ccccc2-c2cccc(N(c3ccc4c5ccccc5n(-c5ccccc5-c5ccccc5)c4c3)c3ccccc3-c3ccccc3)c2)cc1 ; c1ccc(-c2ccccc2-c2cccc(N(c3ccc(-c4ccccc4)c(-c4ccccc4)c3)c3ccc4c5ccccc5n(-c5ccccc5-c5ccccc5)c4c3)c2)cc1]

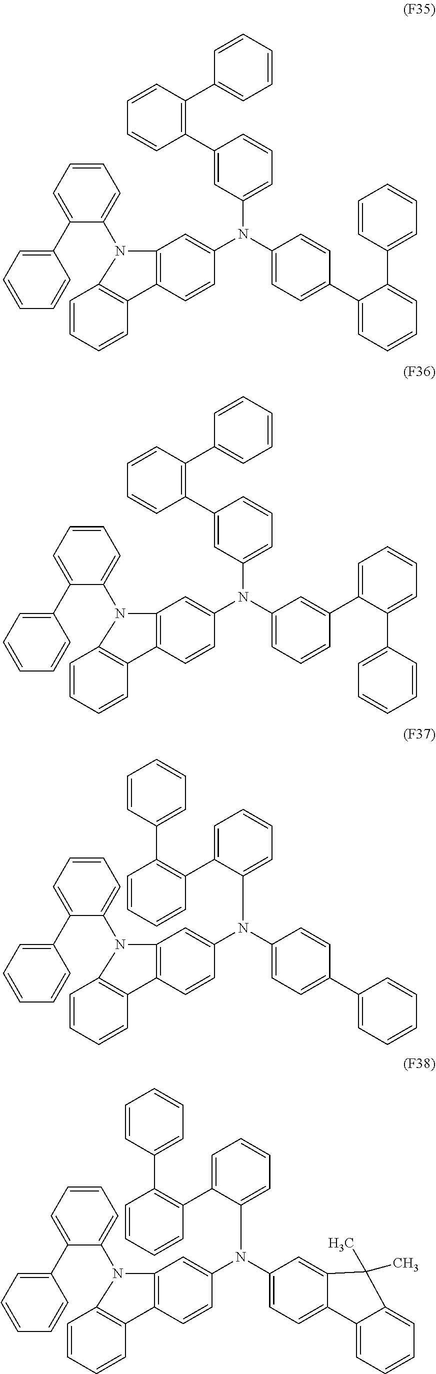 [SMILES: c1ccc(-c2ccccc2-c2ccc(N(c3cccc(-c4ccccc4-c4ccccc4)c3)c3ccc4c5ccccc5n(-c5ccccc5-c5ccccc5)c4c3)cc2)cc1 ; c1ccc(-c2ccccc2-c2cccc(N(c3cccc(-c4ccccc4-c4ccccc4)c3)c3ccc4c5ccccc5n(-c5ccccc5-c5ccccc5)c4c3)c2)cc1 ; c1ccc(-c2ccc(N(c3ccc4c5ccccc5n(-c5ccccc5-c5ccccc5)c4c3)c3ccccc3-c3ccccc3-c3ccccc3)cc2)cc1 ; CC1(C)c2ccccc2-c2ccc(N(c3ccc4c5ccccc5n(-c5ccccc5-c5ccccc5)c4c3)c3ccccc3-c3ccccc3-c3ccccc3)cc21]

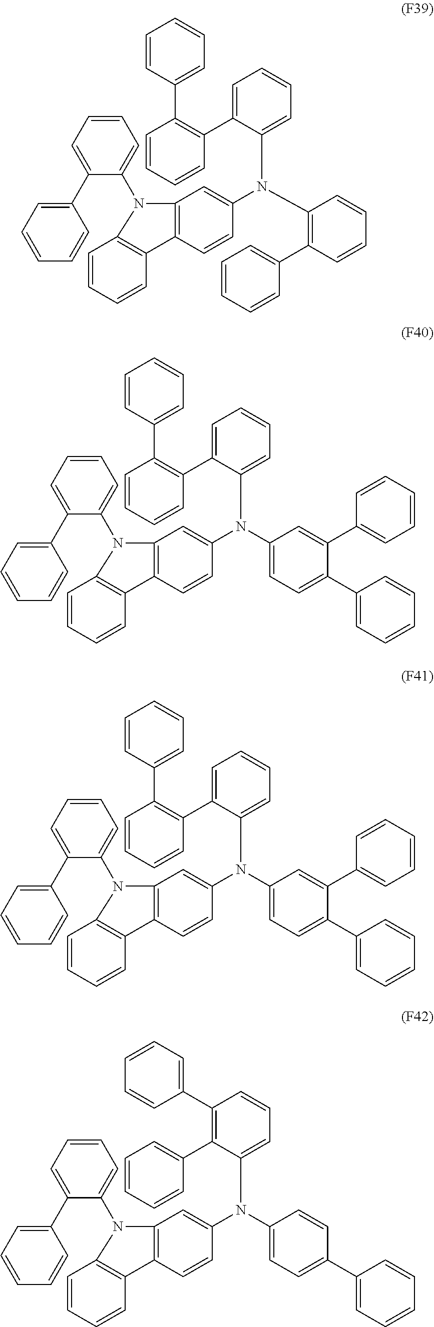 [SMILES: c1ccc(-c2ccccc2-c2ccccc2N(c2ccc3c4ccccc4n(-c4ccccc4-c4ccccc4)c3c2)c2ccccc2-c2ccccc2)cc1 ; c1ccc(-c2ccc(N(c3ccc4c5ccccc5n(-c5ccccc5-c5ccccc5)c4c3)c3ccccc3-c3ccccc3-c3ccccc3)cc2-c2ccccc2)cc1 ; c1ccc(-c2ccc(N(c3ccc4c5ccccc5n(-c5ccccc5-c5ccccc5)c4c3)c3ccccc3-c3ccccc3-c3ccccc3)cc2-c2ccccc2)cc1 ; c1ccc(-c2ccc(N(c3ccc4c5ccccc5n(-c5ccccc5-c5ccccc5)c4c3)c3cccc(-c4ccccc4)c3-c3ccccc3)cc2)cc1]

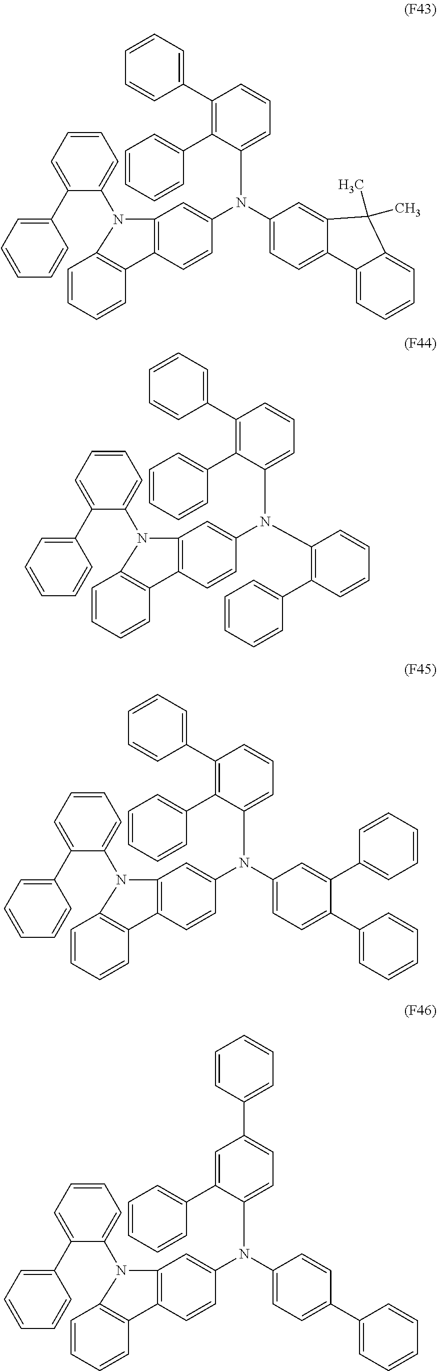 [SMILES: CC1(C)c2ccccc2-c2ccc(N(c3ccc4c5ccccc5n(-c5ccccc5-c5ccccc5)c4c3)c3cccc(-c4ccccc4)c3-c3ccccc3)cc21 ; c1ccc(-c2ccccc2N(c2ccc3c4ccccc4n(-c4ccccc4-c4ccccc4)c3c2)c2cccc(-c3ccccc3)c2-c2ccccc2)cc1 ; c1ccc(-c2ccc(N(c3ccc4c5ccccc5n(-c5ccccc5-c5ccccc5)c4c3)c3cccc(-c4ccccc4)c3-c3ccccc3)cc2-c2ccccc2)cc1 ; c1ccc(-c2ccc(N(c3ccc4c5ccccc5n(-c5ccccc5-c5ccccc5)c4c3)c3ccc(-c4ccccc4)cc3-c3ccccc3)cc2)cc1]

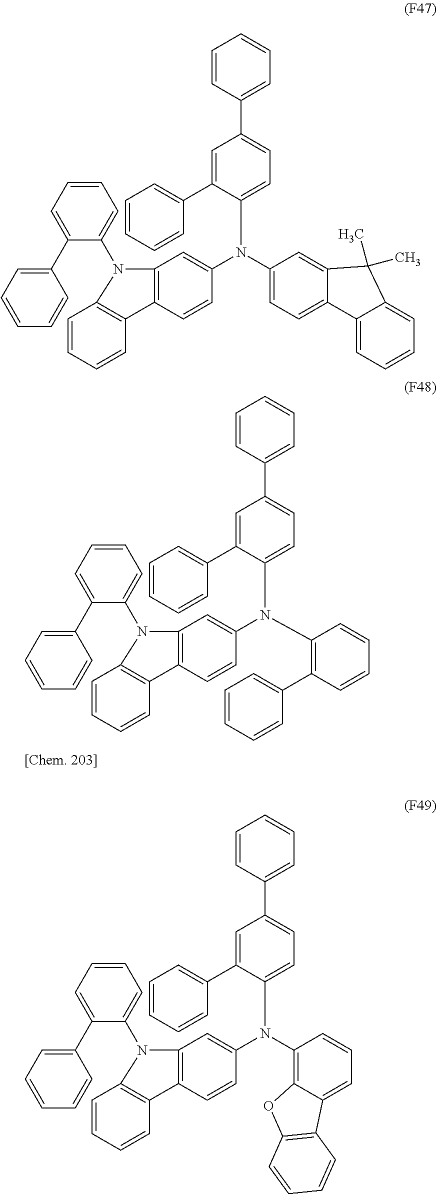 [SMILES: CC1(C)c2ccccc2-c2ccc(N(c3ccc4c5ccccc5n(-c5ccccc5-c5ccccc5)c4c3)c3ccc(-c4ccccc4)cc3-c3ccccc3)cc21 ; c1ccc(-c2ccc(N(c3ccc4c5ccccc5n(-c5ccccc5-c5ccccc5)c4c3)c3ccccc3-c3ccccc3)c(-c3ccccc3)c2)cc1 ; c1ccc(-c2ccc(N(c3ccc4c5ccccc5n(-c5ccccc5-c5ccccc5)c4c3)c3cccc4c3oc3ccccc34)c(-c3ccccc3)c2)cc1]

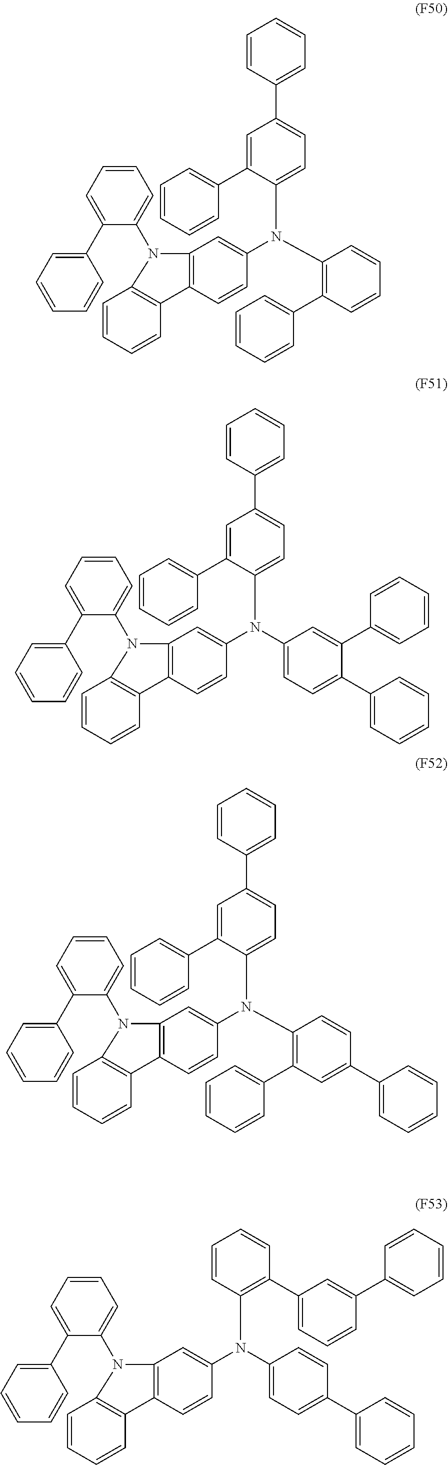 [SMILES: c1ccc(-c2ccc(N(c3ccc4c5ccccc5n(-c5ccccc5-c5ccccc5)c4c3)c3ccccc3-c3ccccc3)c(-c3ccccc3)c2)cc1 ; c1ccc(-c2ccc(N(c3ccc(-c4ccccc4)c(-c4ccccc4)c3)c3ccc4c5ccccc5n(-c5ccccc5-c5ccccc5)c4c3)c(-c3ccccc3)c2)cc1 ; c1ccc(-c2ccc(N(c3ccc4c5ccccc5n(-c5ccccc5-c5ccccc5)c4c3)c3ccc(-c4ccccc4)cc3-c3ccccc3)c(-c3ccccc3)c2)cc1 ; c1ccc(-c2ccc(N(c3ccc4c5ccccc5n(-c5ccccc5-c5ccccc5)c4c3)c3ccccc3-c3cccc(-c4ccccc4)c3)cc2)cc1]

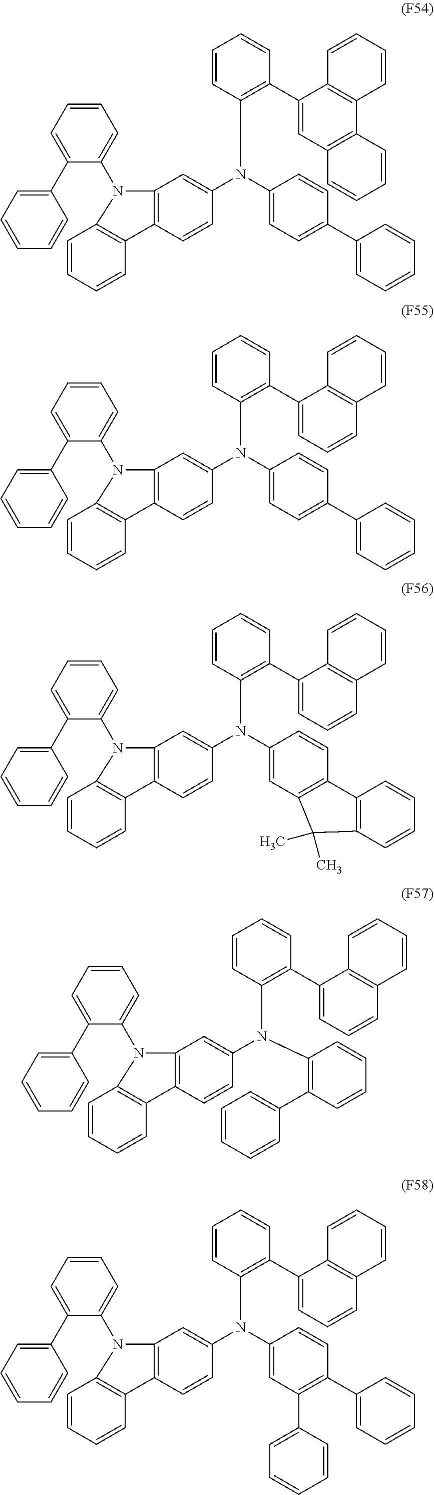 [SMILES: c1ccc(-c2ccc(N(c3ccc4c5ccccc5n(-c5ccccc5-c5ccccc5)c4c3)c3ccccc3-c3cc4ccccc4c4ccccc34)cc2)cc1 ; c1ccc(-c2ccc(N(c3ccc4c5ccccc5n(-c5ccccc5-c5ccccc5)c4c3)c3ccccc3-c3cccc4ccccc34)cc2)cc1 ; CC1(C)c2ccccc2-c2ccc(N(c3ccc4c5ccccc5n(-c5ccccc5-c5ccccc5)c4c3)c3ccccc3-c3cccc4ccccc34)cc21 ; c1ccc(-c2ccccc2N(c2ccc3c4ccccc4n(-c4ccccc4-c4ccccc4)c3c2)c2ccccc2-c2cccc3ccccc23)cc1 ; c1ccc(-c2ccc(N(c3ccc4c5ccccc5n(-c5ccccc5-c5ccccc5)c4c3)c3ccccc3-c3cccc4ccccc34)cc2-c2ccccc2)cc1]

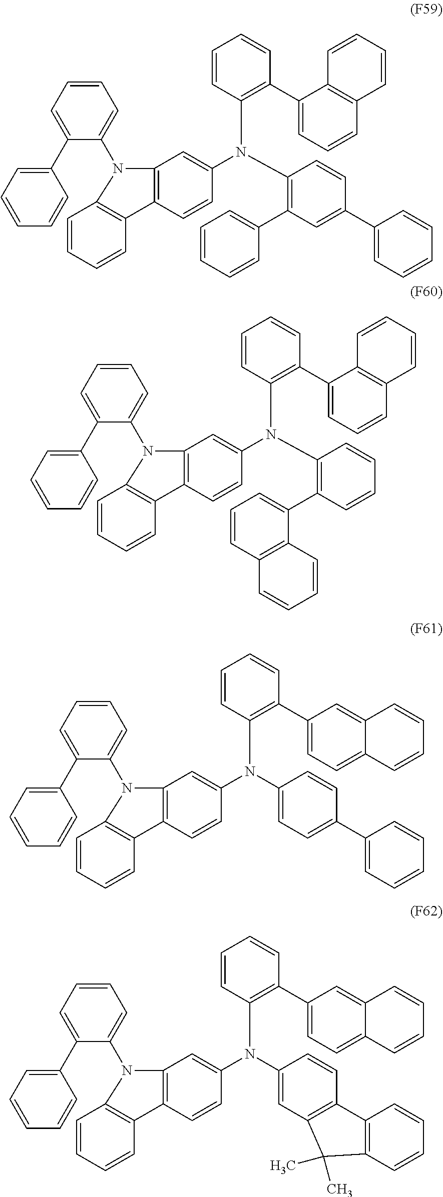

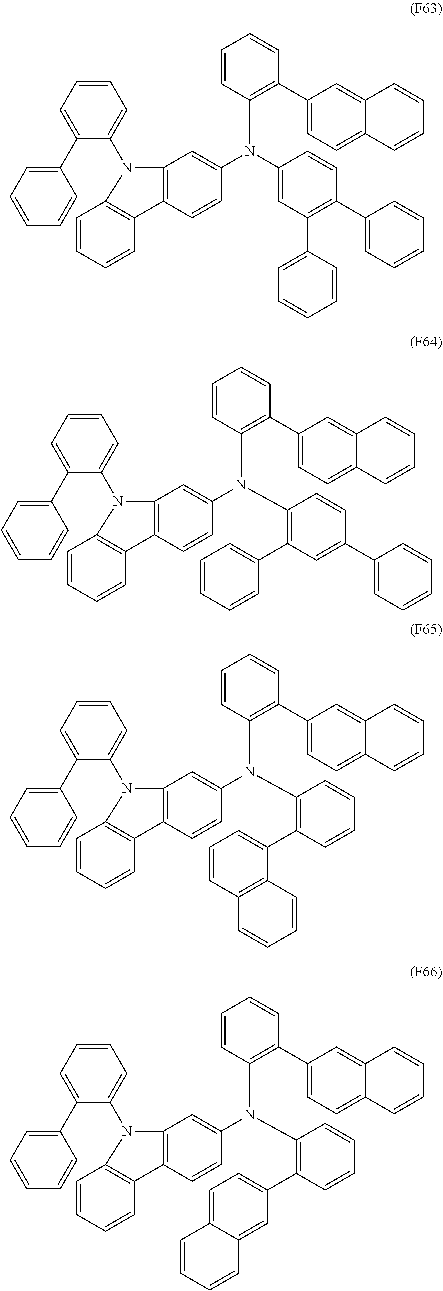 [SMILES: c1ccc(-c2ccc(N(c3ccc4c5ccccc5n(-c5ccccc5-c5ccccc5)c4c3)c3ccccc3-c3ccc4ccccc4c3)cc2-c2ccccc2)cc1 ; c1ccc(-c2ccc(N(c3ccc4c5ccccc5n(-c5ccccc5-c5ccccc5)c4c3)c3ccccc3-c3ccc4ccccc4c3)c(-c3ccccc3)c2)cc1 ; c1ccc(-c2ccccc2-n2c3ccccc3c3ccc(N(c4ccccc4-c4ccc5ccccc5c4)c4ccccc4-c4cccc5ccccc45)cc32)cc1 ; c1ccc(-c2ccccc2-n2c3ccccc3c3ccc(N(c4ccccc4-c4ccc5ccccc5c4)c4ccccc4-c4ccc5ccccc5c4)cc32)cc1]

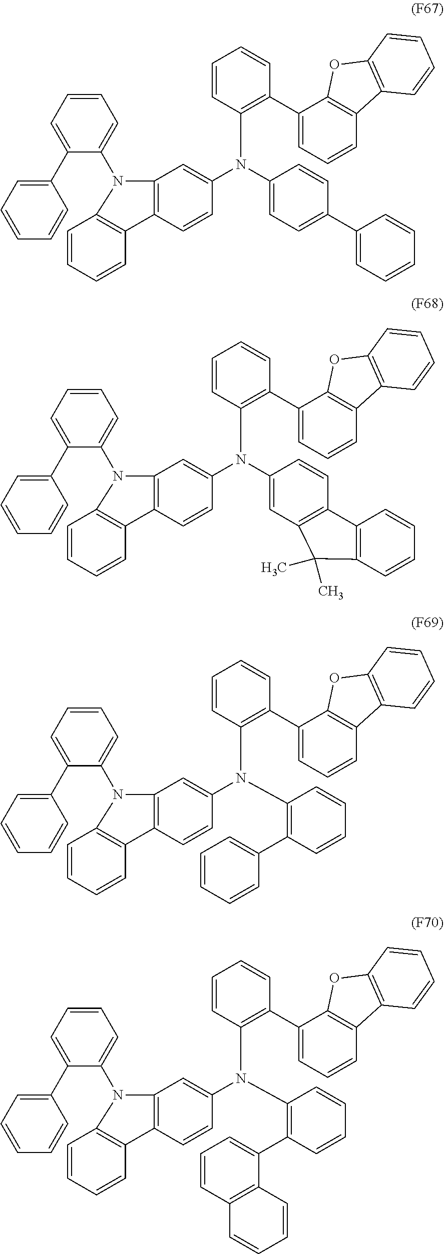 [SMILES: c1ccc(-c2ccc(N(c3ccc4c5ccccc5n(-c5ccccc5-c5ccccc5)c4c3)c3ccccc3-c3cccc4c3oc3ccccc34)cc2)cc1 ; CC1(C)c2ccccc2-c2ccc(N(c3ccc4c5ccccc5n(-c5ccccc5-c5ccccc5)c4c3)c3ccccc3-c3cccc4c3oc3ccccc34)cc21 ; c1ccc(-c2ccccc2N(c2ccc3c4ccccc4n(-c4ccccc4-c4ccccc4)c3c2)c2ccccc2-c2cccc3c2oc2ccccc23)cc1 ; c1ccc(-c2ccccc2-n2c3ccccc3c3ccc(N(c4ccccc4-c4cccc5ccccc45)c4ccccc4-c4cccc5c4oc4ccccc45)cc32)cc1]

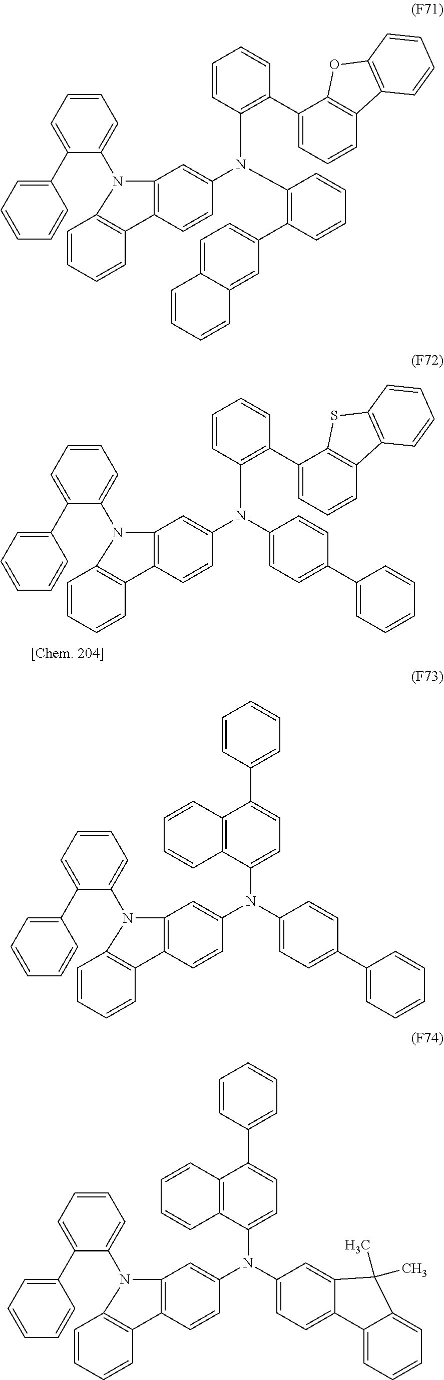 [SMILES: c1ccc(-c2ccccc2-n2c3ccccc3c3ccc(N(c4ccccc4-c4ccc5ccccc5c4)c4ccccc4-c4cccc5c4oc4ccccc45)cc32)cc1 ; c1ccc(-c2ccc(N(c3ccc4c5ccccc5n(-c5ccccc5-c5ccccc5)c4c3)c3ccccc3-c3cccc4c3sc3ccccc34)cc2)cc1 ; c1ccc(-c2ccc(N(c3ccc4c5ccccc5n(-c5ccccc5-c5ccccc5)c4c3)c3ccc(-c4ccccc4)c4ccccc34)cc2)cc1 ; CC1(C)c2ccccc2-c2ccc(N(c3ccc4c5ccccc5n(-c5ccccc5-c5ccccc5)c4c3)c3ccc(-c4ccccc4)c4ccccc34)cc21]

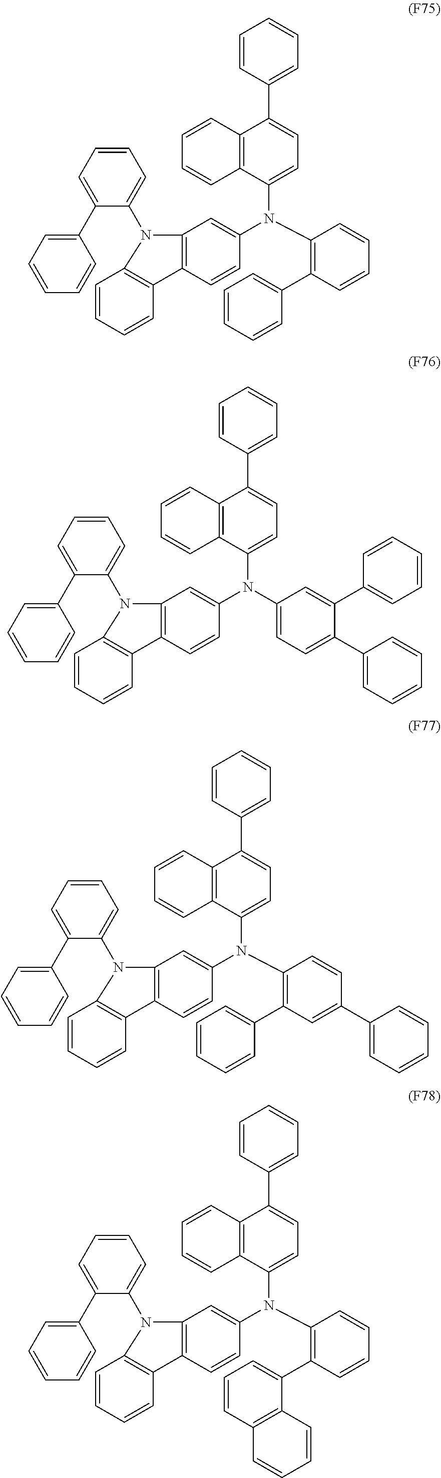 [SMILES: c1ccc(-c2ccccc2N(c2ccc3c4ccccc4n(-c4ccccc4-c4ccccc4)c3c2)c2ccc(-c3ccccc3)c3ccccc23)cc1 ; c1ccc(-c2ccc(N(c3ccc4c5ccccc5n(-c5ccccc5-c5ccccc5)c4c3)c3ccc(-c4ccccc4)c4ccccc34)cc2-c2ccccc2)cc1 ; c1ccc(-c2ccc(N(c3ccc4c5ccccc5n(-c5ccccc5-c5ccccc5)c4c3)c3ccc(-c4ccccc4)c4ccccc34)c(-c3ccccc3)c2)cc1 ; c1ccc(-c2ccccc2-n2c3ccccc3c3ccc(N(c4ccccc4-c4cccc5ccccc45)c4ccc(-c5ccccc5)c5ccccc45)cc32)cc1]

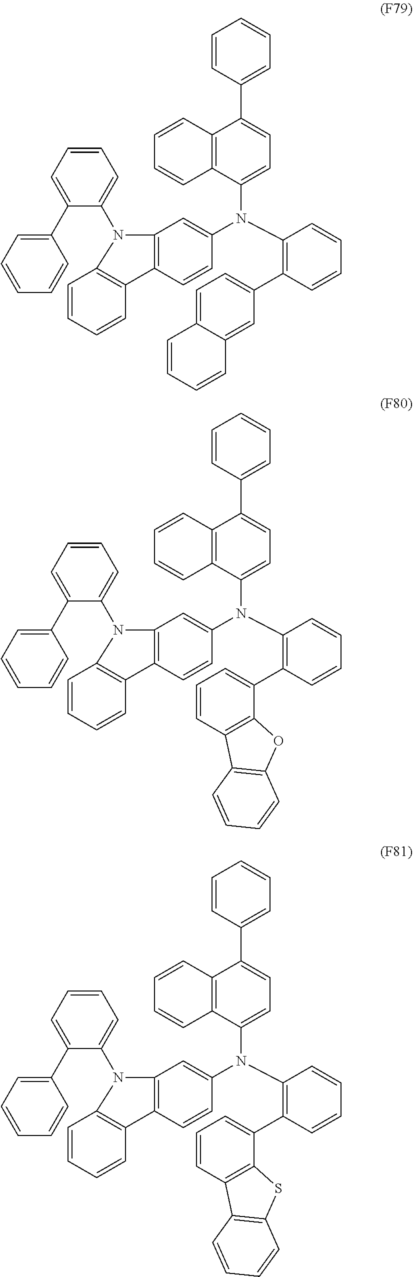

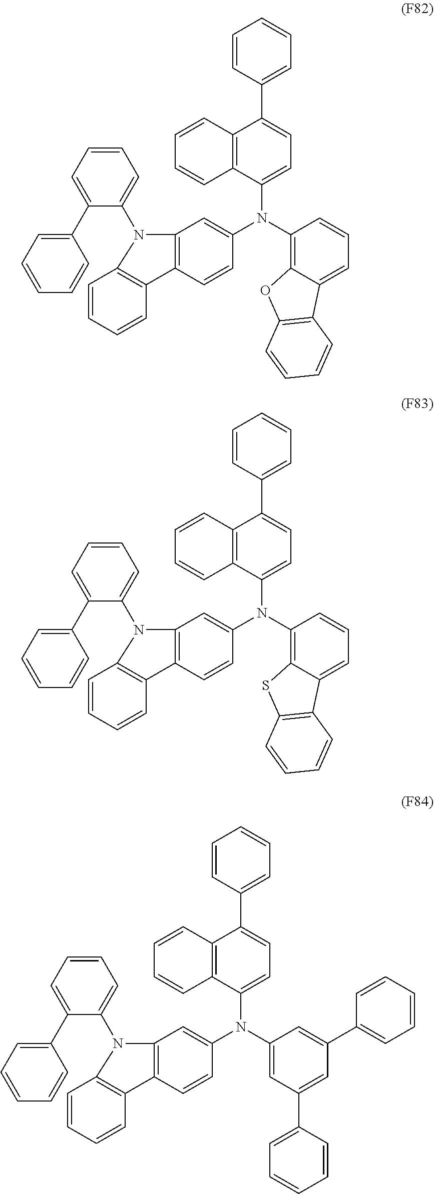

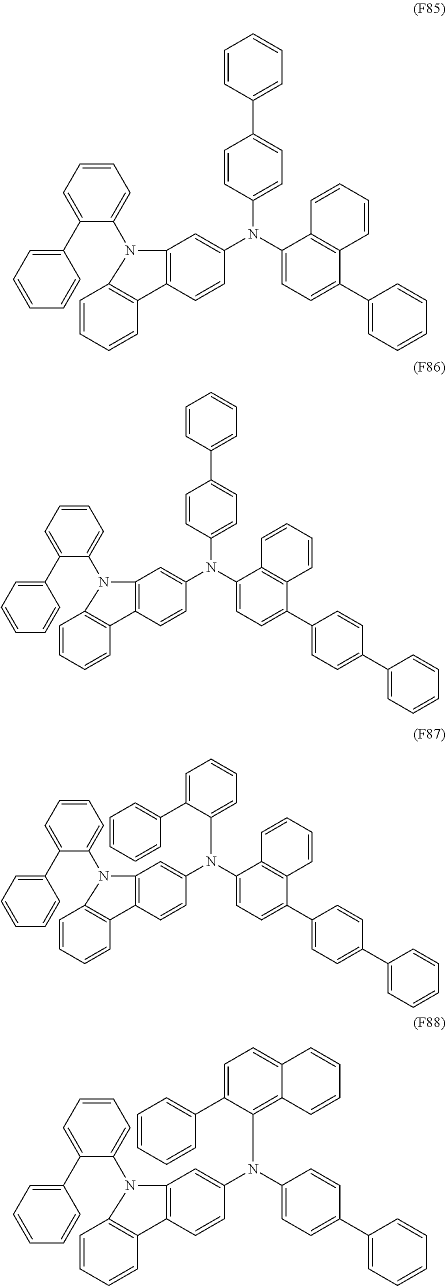 [SMILES: c1ccc(-c2ccc(N(c3ccc4c5ccccc5n(-c5ccccc5-c5ccccc5)c4c3)c3ccc(-c4ccccc4)c4ccccc34)cc2)cc1 ; c1ccc(-c2ccc(-c3ccc(N(c4ccc(-c5ccccc5)cc4)c4ccc5c6ccccc6n(-c6ccccc6-c6ccccc6)c5c4)c4ccccc34)cc2)cc1 ; c1ccc(-c2ccc(-c3ccc(N(c4ccc5c6ccccc6n(-c6ccccc6-c6ccccc6)c5c4)c4ccccc4-c4ccccc4)c4ccccc34)cc2)cc1 ; c1ccc(-c2ccc(N(c3ccc4c5ccccc5n(-c5ccccc5-c5ccccc5)c4c3)c3c(-c4ccccc4)ccc4ccccc34)cc2)cc1]

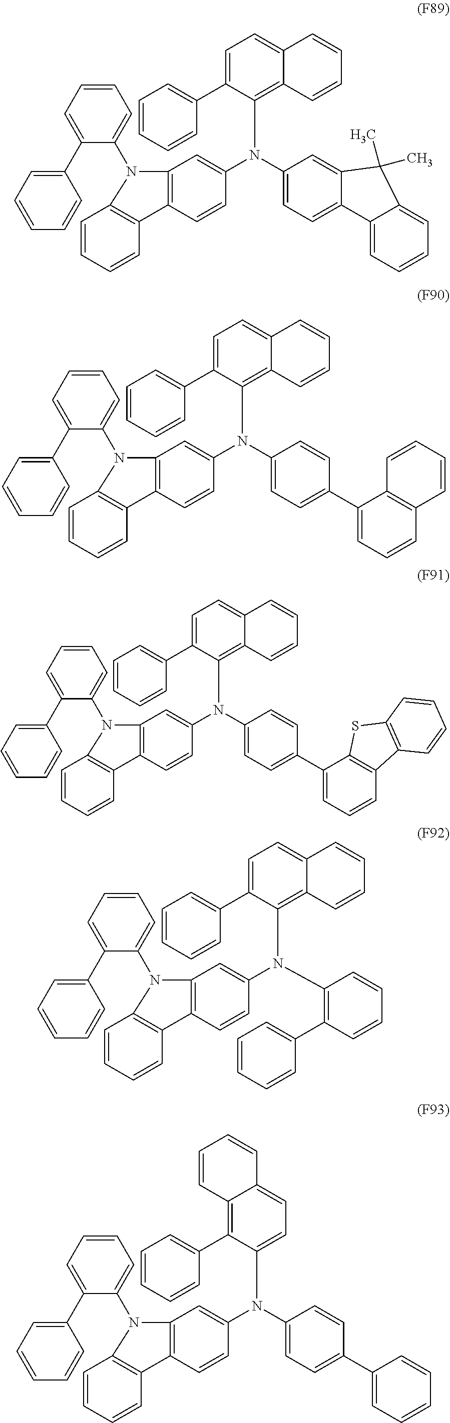

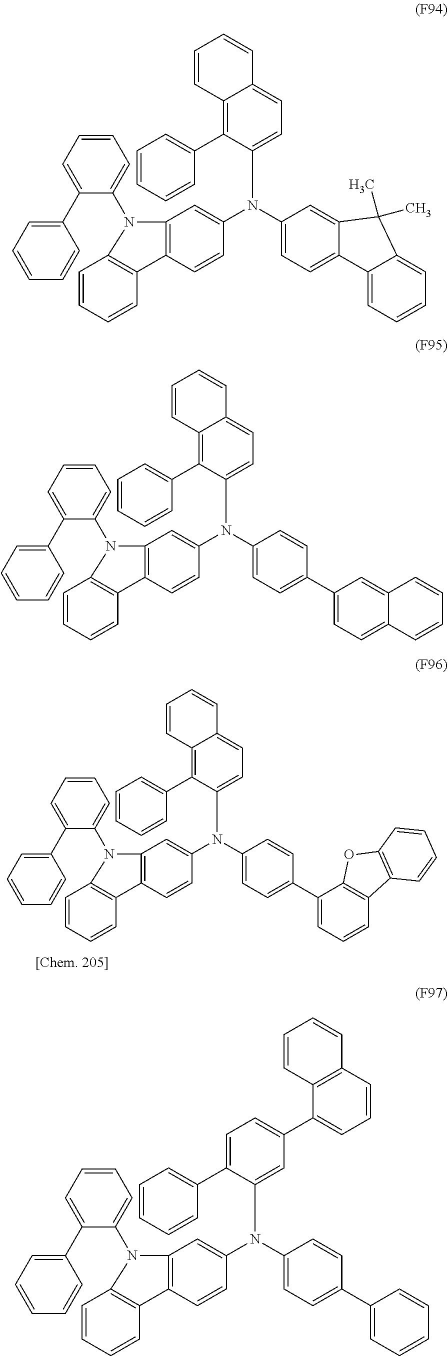 [SMILES: CC1(C)c2ccccc2-c2ccc(N(c3ccc4c5ccccc5n(-c5ccccc5-c5ccccc5)c4c3)c3ccc4ccccc4c3-c3ccccc3)cc21 ; c1ccc(-c2ccccc2-n2c3ccccc3c3ccc(N(c4ccc(-c5ccc6ccccc6c5)cc4)c4ccc5ccccc5c4-c4ccccc4)cc32)cc1 ; c1ccc(-c2ccccc2-n2c3ccccc3c3ccc(N(c4ccc(-c5cccc6c5oc5ccccc56)cc4)c4ccc5ccccc5c4-c4ccccc4)cc32)cc1 ; c1ccc(-c2ccc(N(c3ccc4c5ccccc5n(-c5ccccc5-c5ccccc5)c4c3)c3cc(-c4cccc5ccccc45)ccc3-c3ccccc3)cc2)cc1]

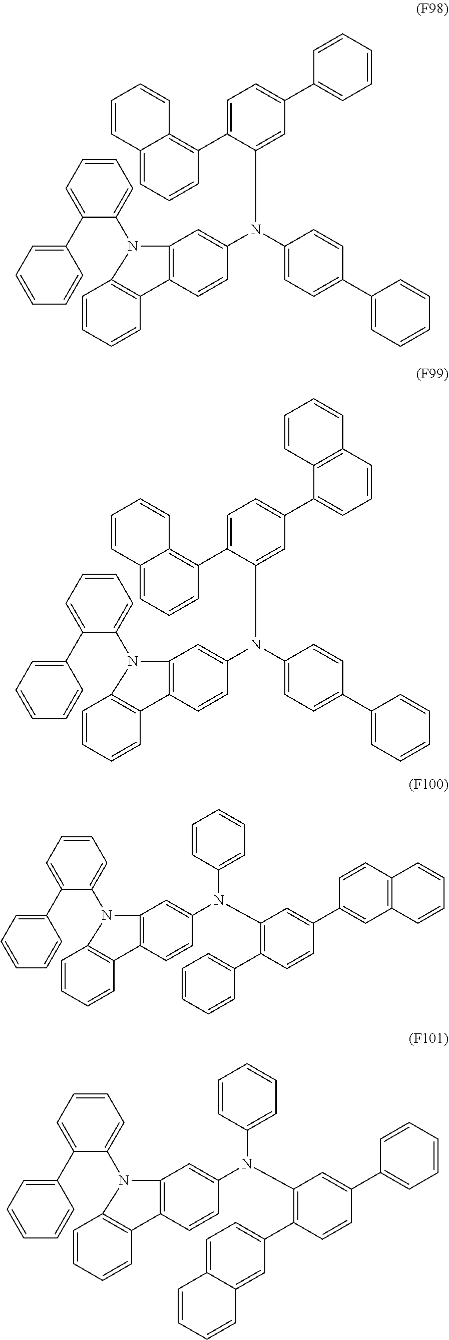 [SMILES: c1ccc(-c2ccc(N(c3ccc4c5ccccc5n(-c5ccccc5-c5ccccc5)c4c3)c3cc(-c4ccccc4)ccc3-c3cccc4ccccc34)cc2)cc1 ; c1ccc(-c2ccc(N(c3ccc4c5ccccc5n(-c5ccccc5-c5ccccc5)c4c3)c3cc(-c4cccc5ccccc45)ccc3-c3cccc4ccccc34)cc2)cc1 ; c1ccc(-c2ccc(-c3ccc4ccccc4c3)cc2N(c2ccccc2)c2ccc3c4ccccc4n(-c4ccccc4-c4ccccc4)c3c2)cc1 ; c1ccc(-c2ccc(-c3ccc4ccccc4c3)c(N(c3ccccc3)c3ccc4c5ccccc5n(-c5ccccc5-c5ccccc5)c4c3)c2)cc1]

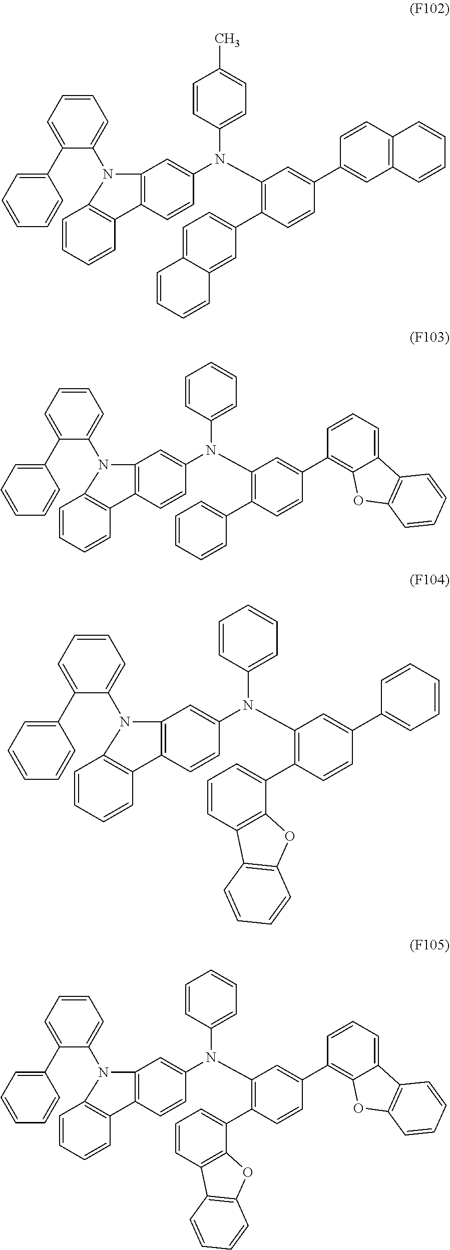 [SMILES: Cc1ccc(N(c2ccc3c4ccccc4n(-c4ccccc4-c4ccccc4)c3c2)c2cc(-c3ccc4ccccc4c3)ccc2-c2ccc3ccccc3c2)cc1 ; c1ccc(-c2ccc(-c3cccc4c3oc3ccccc34)cc2N(c2ccccc2)c2ccc3c4ccccc4n(-c4ccccc4-c4ccccc4)c3c2)cc1 ; c1ccc(-c2ccc(-c3cccc4c3oc3ccccc34)c(N(c3ccccc3)c3ccc4c5ccccc5n(-c5ccccc5-c5ccccc5)c4c3)c2)cc1 ; c1ccc(-c2ccccc2-n2c3ccccc3c3ccc(N(c4ccccc4)c4cc(-c5cccc6c5oc5ccccc56)ccc4-c4cccc5c4oc4ccccc45)cc32)cc1]

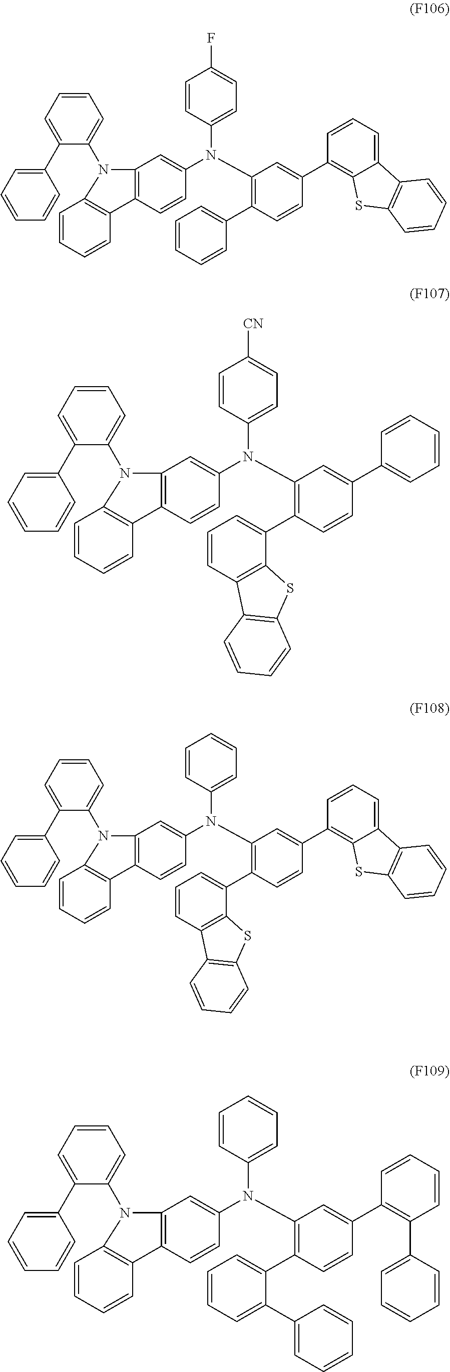 [SMILES: Fc1ccc(N(c2ccc3c4ccccc4n(-c4ccccc4-c4ccccc4)c3c2)c2cc(-c3cccc4c3sc3ccccc34)ccc2-c2ccccc2)cc1 ; N#Cc1ccc(N(c2ccc3c4ccccc4n(-c4ccccc4-c4ccccc4)c3c2)c2cc(-c3ccccc3)ccc2-c2cccc3c2sc2ccccc23)cc1 ; c1ccc(-c2ccccc2-n2c3ccccc3c3ccc(N(c4ccccc4)c4cc(-c5cccc6c5sc5ccccc56)ccc4-c4cccc5c4sc4ccccc45)cc32)cc1 ; c1ccc(-c2ccccc2-c2ccc(-c3ccccc3-c3ccccc3)c(N(c3ccccc3)c3ccc4c5ccccc5n(-c5ccccc5-c5ccccc5)c4c3)c2)cc1]

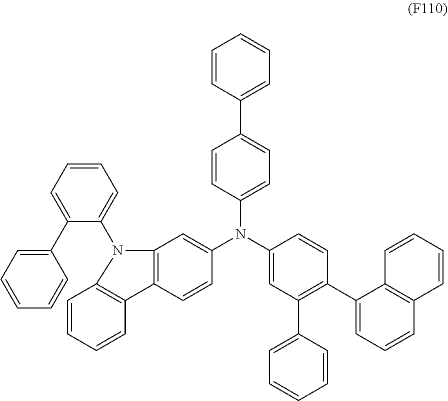 [SMILES: c1ccc(-c2ccc(N(c3ccc(-c4cccc5ccccc45)c(-c4ccccc4)c3)c3ccc4c5ccccc5n(-c5ccccc5-c5ccccc5)c4c3)cc2)cc1]

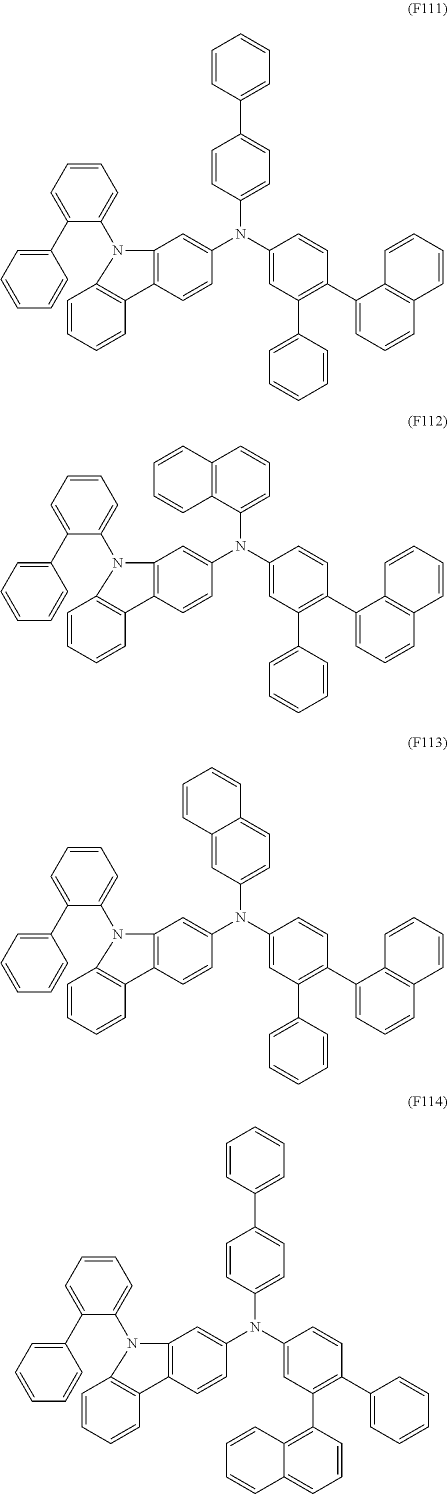 [SMILES: c1ccc(-c2ccc(N(c3ccc(-c4cccc5ccccc45)c(-c4ccccc4)c3)c3ccc4c5ccccc5n(-c5ccccc5-c5ccccc5)c4c3)cc2)cc1 ; c1ccc(-c2cc(N(c3ccc4c5ccccc5n(-c5ccccc5-c5ccccc5)c4c3)c3cccc4ccccc34)ccc2-c2cccc3ccccc23)cc1 ; c1ccc(-c2cc(N(c3ccc4ccccc4c3)c3ccc4c5ccccc5n(-c5ccccc5-c5ccccc5)c4c3)ccc2-c2cccc3ccccc23)cc1 ; c1ccc(-c2ccc(N(c3ccc(-c4ccccc4)c(-c4cccc5ccccc45)c3)c3ccc4c5ccccc5n(-c5ccccc5-c5ccccc5)c4c3)cc2)cc1]

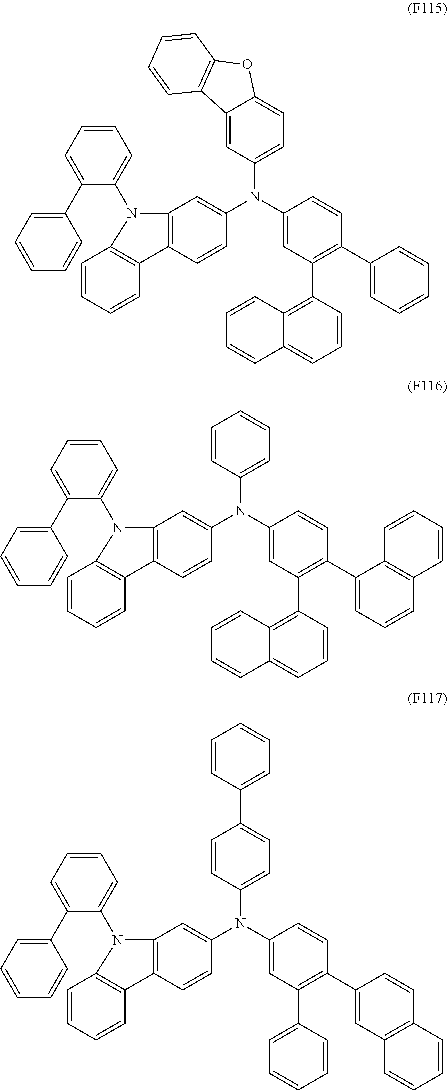

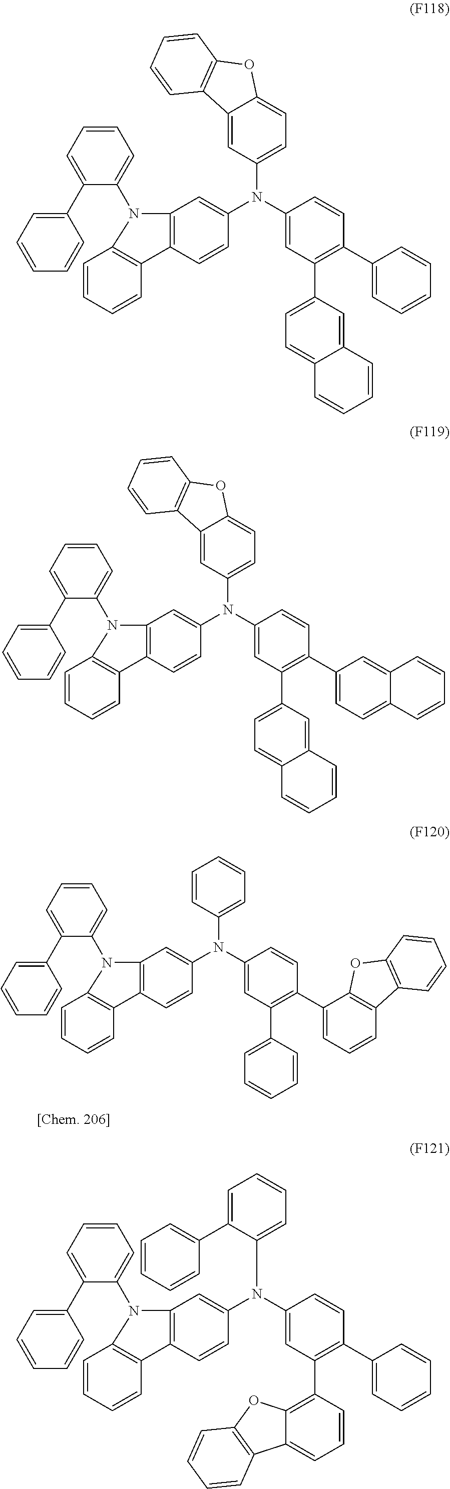 [SMILES: c1ccc(-c2ccc(N(c3ccc4oc5ccccc5c4c3)c3ccc4c5ccccc5n(-c5ccccc5-c5ccccc5)c4c3)cc2-c2ccc3ccccc3c2)cc1 ; c1ccc(-c2ccccc2-n2c3ccccc3c3ccc(N(c4ccc(-c5ccc6ccccc6c5)c(-c5ccc6ccccc6c5)c4)c4ccc5oc6ccccc6c5c4)cc32)cc1 ; c1ccc(-c2cc(N(c3ccccc3)c3ccc4c5ccccc5n(-c5ccccc5-c5ccccc5)c4c3)ccc2-c2cccc3c2oc2ccccc23)cc1 ; c1ccc(-c2ccc(N(c3ccc4c5ccccc5n(-c5ccccc5-c5ccccc5)c4c3)c3ccccc3-c3ccccc3)cc2-c2cccc3c2oc2ccccc23)cc1]

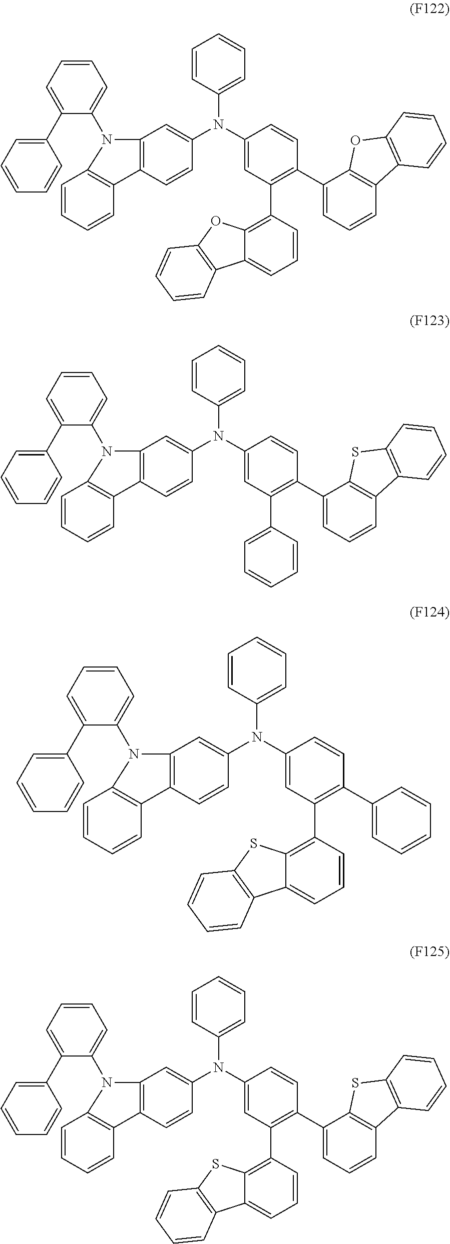 [SMILES: c1ccc(-c2ccccc2-n2c3ccccc3c3ccc(N(c4ccccc4)c4ccc(-c5cccc6c5oc5ccccc56)c(-c5cccc6c5oc5ccccc56)c4)cc32)cc1 ; c1ccc(-c2cc(N(c3ccccc3)c3ccc4c5ccccc5n(-c5ccccc5-c5ccccc5)c4c3)ccc2-c2cccc3c2sc2ccccc23)cc1 ; c1ccc(-c2ccc(N(c3ccccc3)c3ccc4c5ccccc5n(-c5ccccc5-c5ccccc5)c4c3)cc2-c2cccc3c2sc2ccccc23)cc1 ; c1ccc(-c2ccccc2-n2c3ccccc3c3ccc(N(c4ccccc4)c4ccc(-c5cccc6c5sc5ccccc56)c(-c5cccc6c5sc5ccccc56)c4)cc32)cc1]

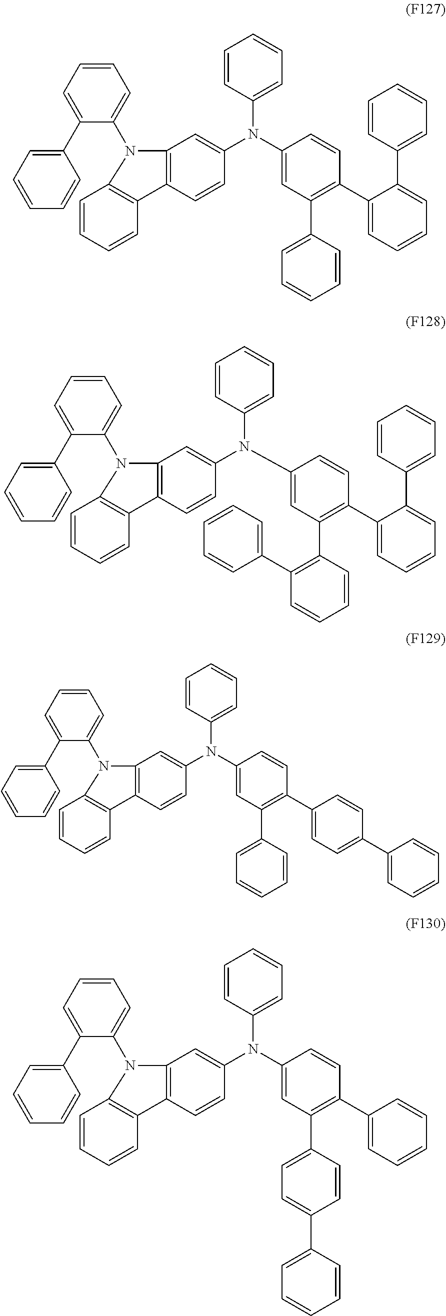 [SMILES: c1ccc(-c2ccccc2-c2ccc(N(c3ccccc3)c3ccc4c5ccccc5n(-c5ccccc5-c5ccccc5)c4c3)cc2-c2ccccc2)cc1 ; c1ccc(-c2ccccc2-c2ccc(N(c3ccccc3)c3ccc4c5ccccc5n(-c5ccccc5-c5ccccc5)c4c3)cc2-c2ccccc2-c2ccccc2)cc1 ; c1ccc(-c2ccc(-c3ccc(N(c4ccccc4)c4ccc5c6ccccc6n(-c6ccccc6-c6ccccc6)c5c4)cc3-c3ccccc3)cc2)cc1 ; c1ccc(-c2ccc(-c3cc(N(c4ccccc4)c4ccc5c6ccccc6n(-c6ccccc6-c6ccccc6)c5c4)ccc3-c3ccccc3)cc2)cc1]

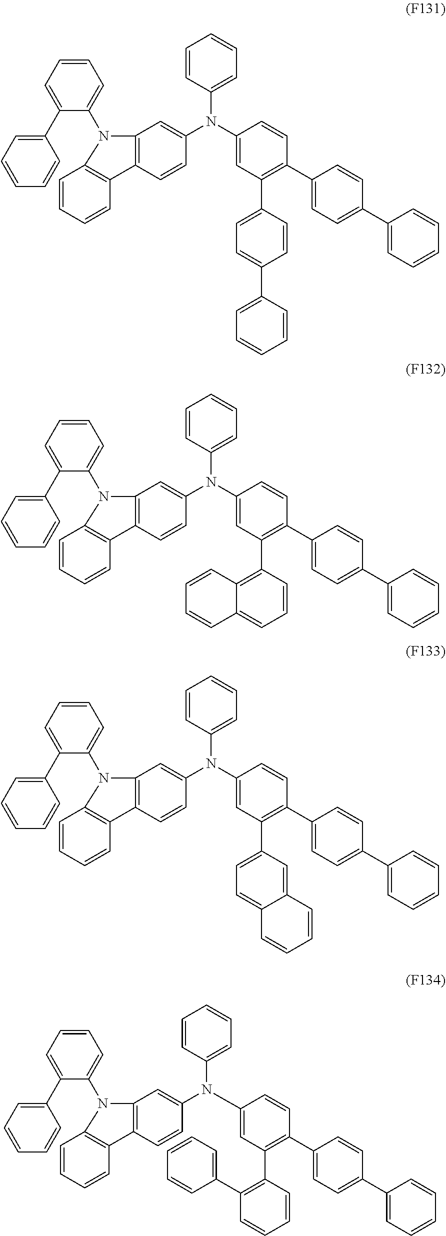 [SMILES: c1ccc(-c2ccc(-c3ccc(N(c4ccccc4)c4ccc5c6ccccc6n(-c6ccccc6-c6ccccc6)c5c4)cc3-c3ccc(-c4ccccc4)cc3)cc2)cc1 ; c1ccc(-c2ccc(-c3ccc(N(c4ccccc4)c4ccc5c6ccccc6n(-c6ccccc6-c6ccccc6)c5c4)cc3-c3cccc4ccccc34)cc2)cc1 ; c1ccc(-c2ccc(-c3ccc(N(c4ccccc4)c4ccc5c6ccccc6n(-c6ccccc6-c6ccccc6)c5c4)cc3-c3ccc4ccccc4c3)cc2)cc1 ; c1ccc(-c2ccc(-c3ccc(N(c4ccccc4)c4ccc5c6ccccc6n(-c6ccccc6-c6ccccc6)c5c4)cc3-c3ccccc3-c3ccccc3)cc2)cc1]

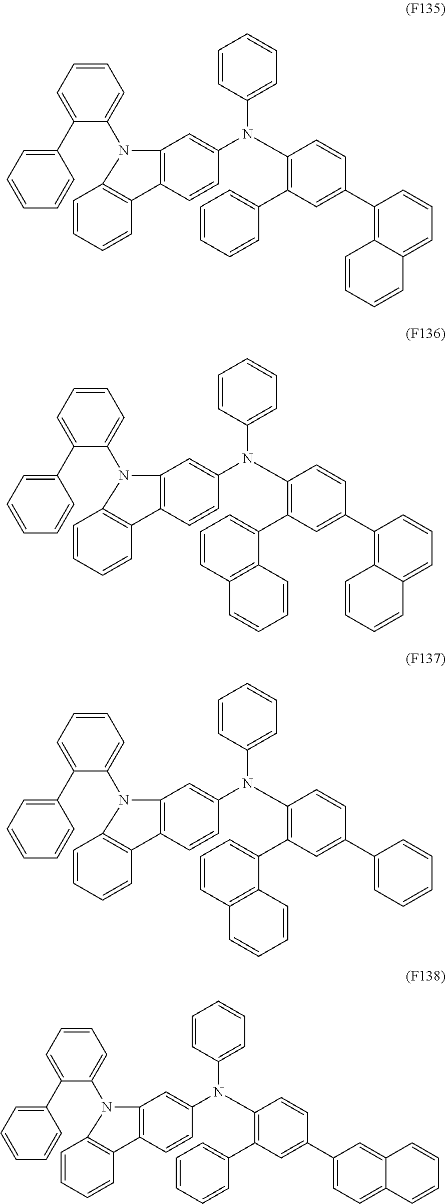 [SMILES: c1ccc(-c2cc(-c3cccc4ccccc34)ccc2N(c2ccccc2)c2ccc3c4ccccc4n(-c4ccccc4-c4ccccc4)c3c2)cc1 ; c1ccc(-c2ccccc2-n2c3ccccc3c3ccc(N(c4ccccc4)c4ccc(-c5cccc6ccccc56)cc4-c4cccc5ccccc45)cc32)cc1 ; c1ccc(-c2ccc(N(c3ccccc3)c3ccc4c5ccccc5n(-c5ccccc5-c5ccccc5)c4c3)c(-c3cccc4ccccc34)c2)cc1 ; c1ccc(-c2cc(-c3ccc4ccccc4c3)ccc2N(c2ccccc2)c2ccc3c4ccccc4n(-c4ccccc4-c4ccccc4)c3c2)cc1]

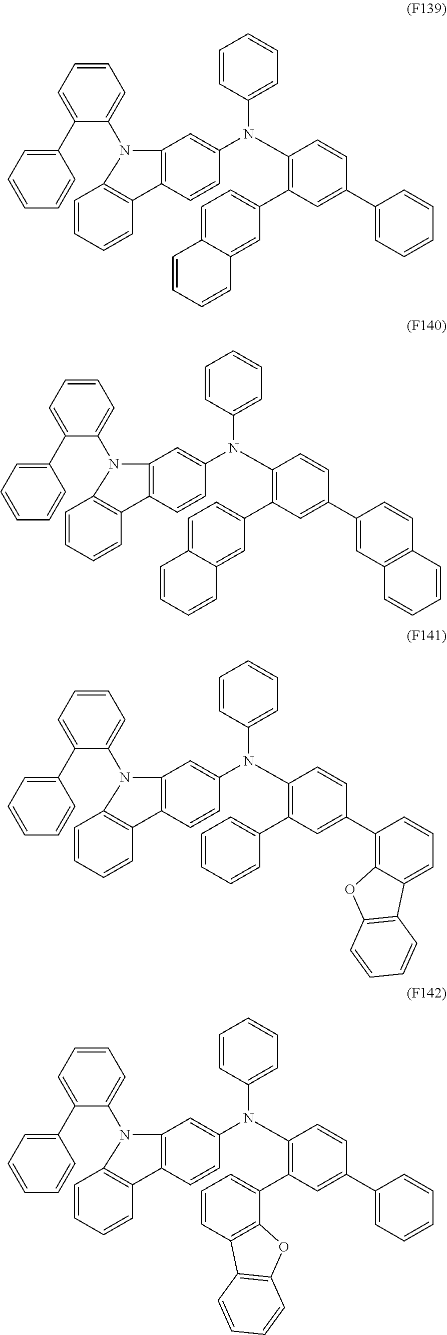 [SMILES: c1ccc(-c2ccc(N(c3ccccc3)c3ccc4c5ccccc5n(-c5ccccc5-c5ccccc5)c4c3)c(-c3ccc4ccccc4c3)c2)cc1 ; c1ccc(-c2ccccc2-n2c3ccccc3c3ccc(N(c4ccccc4)c4ccc(-c5ccc6ccccc6c5)cc4-c4ccc5ccccc5c4)cc32)cc1 ; c1ccc(-c2cc(-c3cccc4c3oc3ccccc34)ccc2N(c2ccccc2)c2ccc3c4ccccc4n(-c4ccccc4-c4ccccc4)c3c2)cc1 ; c1ccc(-c2ccc(N(c3ccccc3)c3ccc4c5ccccc5n(-c5ccccc5-c5ccccc5)c4c3)c(-c3cccc4c3oc3ccccc34)c2)cc1]

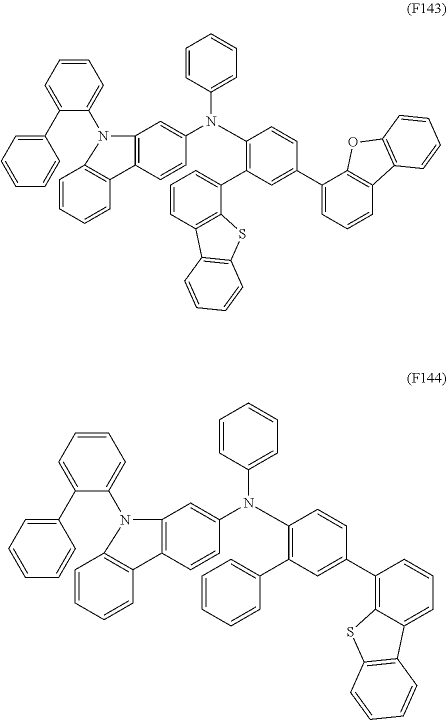 [SMILES: c1ccc(-c2ccccc2-n2c3ccccc3c3ccc(N(c4ccccc4)c4ccc(-c5cccc6c5oc5ccccc56)cc4-c4cccc5c4sc4ccccc45)cc32)cc1 ; c1ccc(-c2cc(-c3cccc4c3sc3ccccc34)ccc2N(c2ccccc2)c2ccc3c4ccccc4n(-c4ccccc4-c4ccccc4)c3c2)cc1]

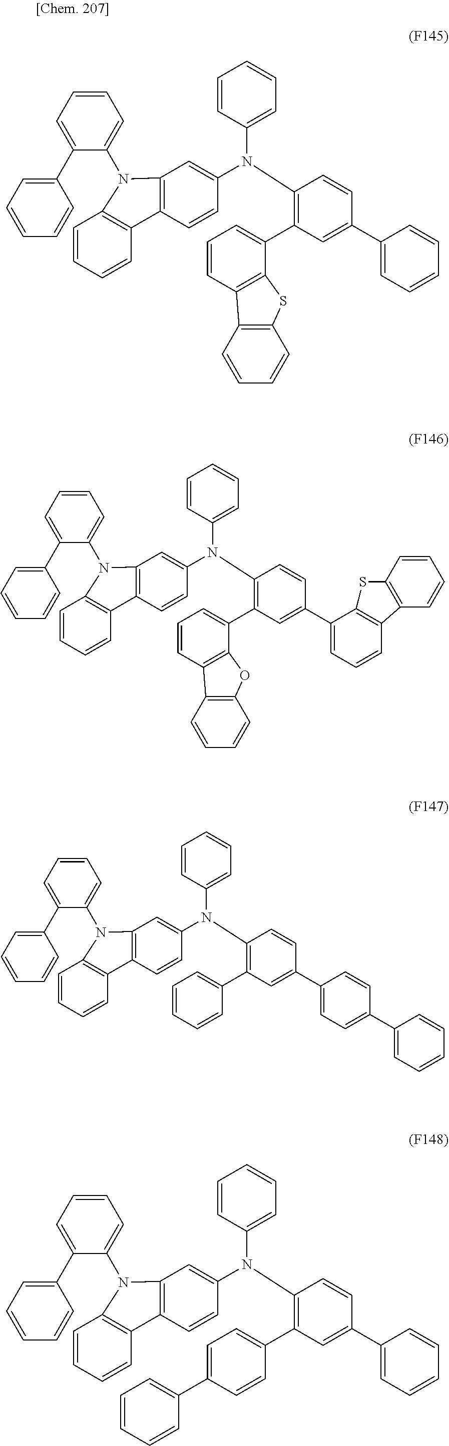 [SMILES: c1ccc(-c2ccc(N(c3ccccc3)c3ccc4c5ccccc5n(-c5ccccc5-c5ccccc5)c4c3)c(-c3cccc4c3sc3ccccc34)c2)cc1 ; c1ccc(-c2ccccc2-n2c3ccccc3c3ccc(N(c4ccccc4)c4ccc(-c5cccc6c5sc5ccccc56)cc4-c4cccc5c4oc4ccccc45)cc32)cc1 ; c1ccc(-c2ccc(-c3ccc(N(c4ccccc4)c4ccc5c6ccccc6n(-c6ccccc6-c6ccccc6)c5c4)c(-c4ccccc4)c3)cc2)cc1 ; c1ccc(-c2ccc(-c3cc(-c4ccccc4)ccc3N(c3ccccc3)c3ccc4c5ccccc5n(-c5ccccc5-c5ccccc5)c4c3)cc2)cc1]

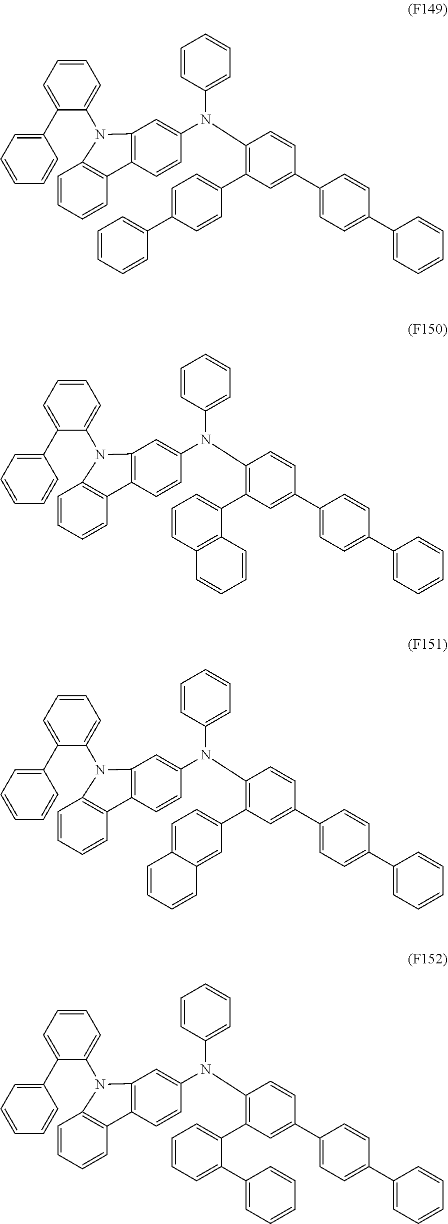 [SMILES: c1ccc(-c2ccc(-c3ccc(N(c4ccccc4)c4ccc5c6ccccc6n(-c6ccccc6-c6ccccc6)c5c4)c(-c4ccc(-c5ccccc5)cc4)c3)cc2)cc1 ; c1ccc(-c2ccc(-c3ccc(N(c4ccccc4)c4ccc5c6ccccc6n(-c6ccccc6-c6ccccc6)c5c4)c(-c4cccc5ccccc45)c3)cc2)cc1 ; c1ccc(-c2ccc(-c3ccc(N(c4ccccc4)c4ccc5c6ccccc6n(-c6ccccc6-c6ccccc6)c5c4)c(-c4ccc5ccccc5c4)c3)cc2)cc1 ; c1ccc(-c2ccc(-c3ccc(N(c4ccccc4)c4ccc5c6ccccc6n(-c6ccccc6-c6ccccc6)c5c4)c(-c4ccccc4-c4ccccc4)c3)cc2)cc1]

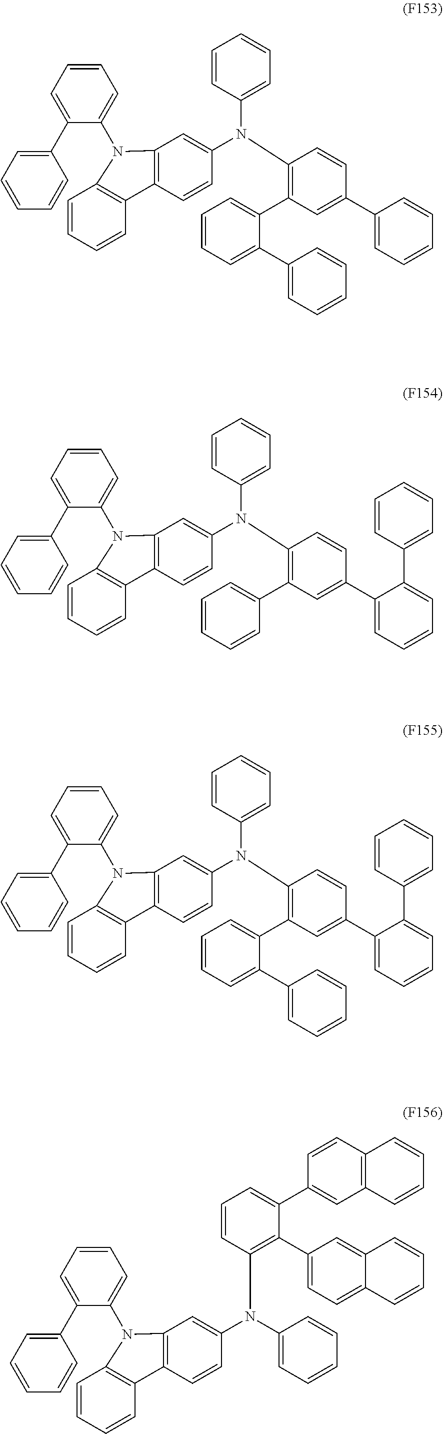 [SMILES: c1ccc(-c2ccc(N(c3ccccc3)c3ccc4c5ccccc5n(-c5ccccc5-c5ccccc5)c4c3)c(-c3ccccc3-c3ccccc3)c2)cc1 ; c1ccc(-c2ccccc2-c2ccc(N(c3ccccc3)c3ccc4c5ccccc5n(-c5ccccc5-c5ccccc5)c4c3)c(-c3ccccc3)c2)cc1 ; c1ccc(-c2ccccc2-c2ccc(N(c3ccccc3)c3ccc4c5ccccc5n(-c5ccccc5-c5ccccc5)c4c3)c(-c3ccccc3-c3ccccc3)c2)cc1 ; c1ccc(-c2ccccc2-n2c3ccccc3c3ccc(N(c4ccccc4)c4cccc(-c5ccc6ccccc6c5)c4-c4ccc5ccccc5c4)cc32)cc1]

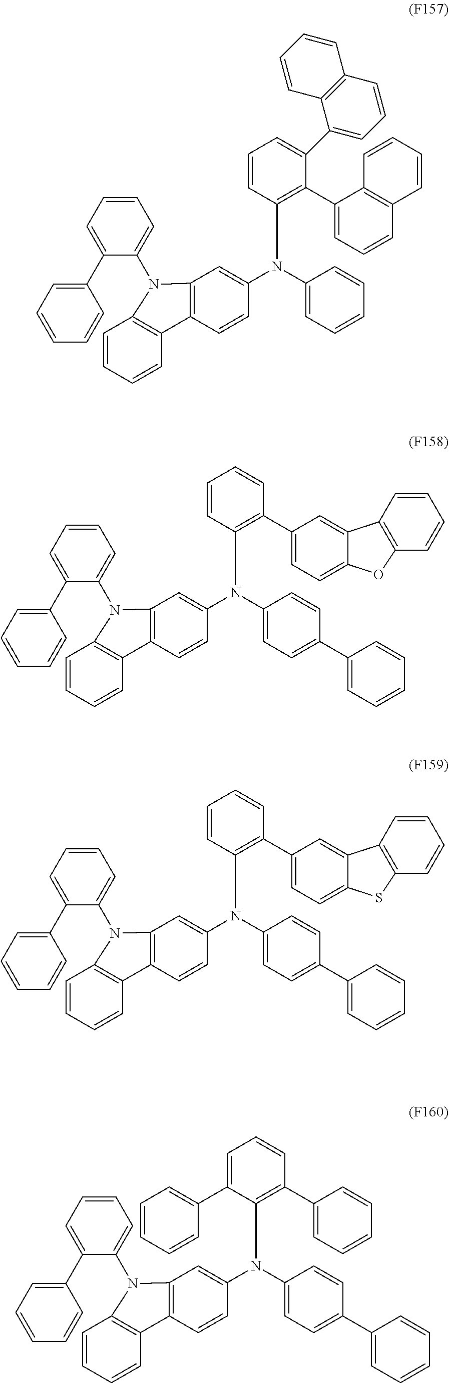 [SMILES: c1ccc(-c2ccccc2-n2c3ccccc3c3ccc(N(c4ccccc4)c4cccc(-c5cccc6ccccc56)c4-c4cccc5ccccc45)cc32)cc1 ; c1ccc(-c2ccc(N(c3ccc4c5ccccc5n(-c5ccccc5-c5ccccc5)c4c3)c3ccccc3-c3ccc4oc5ccccc5c4c3)cc2)cc1 ; c1ccc(-c2ccc(N(c3ccc4c5ccccc5n(-c5ccccc5-c5ccccc5)c4c3)c3ccccc3-c3ccc4sc5ccccc5c4c3)cc2)cc1 ; c1ccc(-c2ccc(N(c3ccc4c5ccccc5n(-c5ccccc5-c5ccccc5)c4c3)c3c(-c4ccccc4)cccc3-c3ccccc3)cc2)cc1]

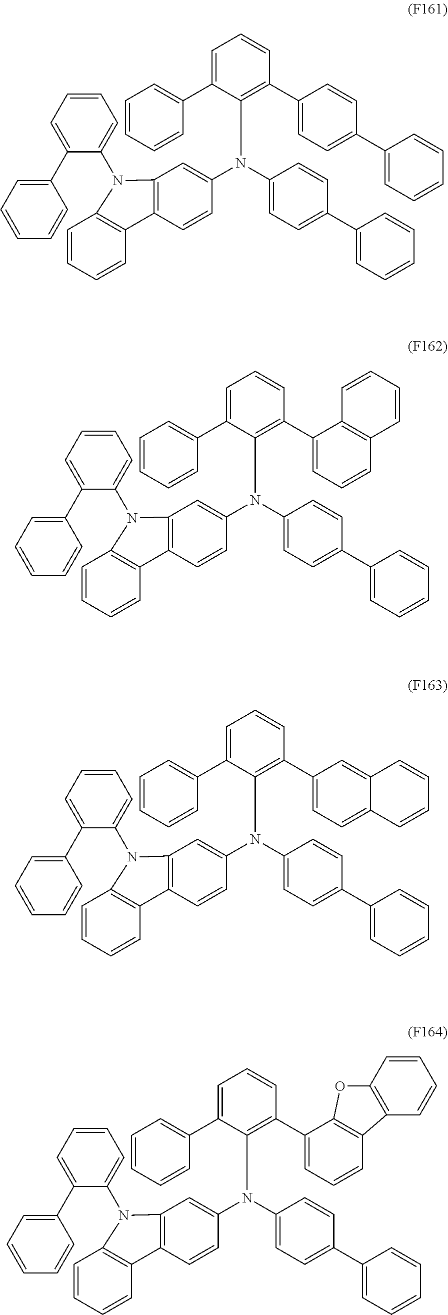 [SMILES: c1ccc(-c2ccc(-c3cccc(-c4ccccc4)c3N(c3ccc(-c4ccccc4)cc3)c3ccc4c5ccccc5n(-c5ccccc5-c5ccccc5)c4c3)cc2)cc1 ; c1ccc(-c2ccc(N(c3ccc4c5ccccc5n(-c5ccccc5-c5ccccc5)c4c3)c3c(-c4ccccc4)cccc3-c3cccc4ccccc34)cc2)cc1 ; c1ccc(-c2ccc(N(c3ccc4c5ccccc5n(-c5ccccc5-c5ccccc5)c4c3)c3c(-c4ccccc4)cccc3-c3ccc4ccccc4c3)cc2)cc1 ; c1ccc(-c2ccc(N(c3ccc4c5ccccc5n(-c5ccccc5-c5ccccc5)c4c3)c3c(-c4ccccc4)cccc3-c3cccc4c3oc3ccccc34)cc2)cc1]

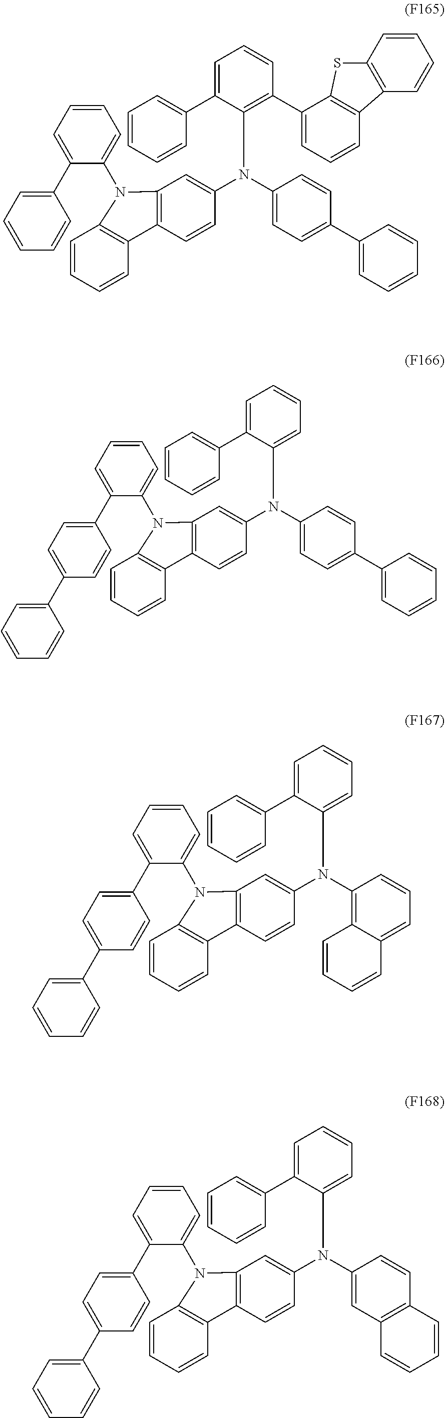 [SMILES: c1ccc(-c2ccc(N(c3ccc4c5ccccc5n(-c5ccccc5-c5ccccc5)c4c3)c3c(-c4ccccc4)cccc3-c3cccc4c3sc3ccccc34)cc2)cc1 ; c1ccc(-c2ccc(-c3ccccc3-n3c4ccccc4c4ccc(N(c5ccc(-c6ccccc6)cc5)c5ccccc5-c5ccccc5)cc43)cc2)cc1 ; c1ccc(-c2ccc(-c3ccccc3-n3c4ccccc4c4ccc(N(c5ccccc5-c5ccccc5)c5cccc6ccccc56)cc43)cc2)cc1 ; c1ccc(-c2ccc(-c3ccccc3-n3c4ccccc4c4ccc(N(c5ccc6ccccc6c5)c5ccccc5-c5ccccc5)cc43)cc2)cc1]

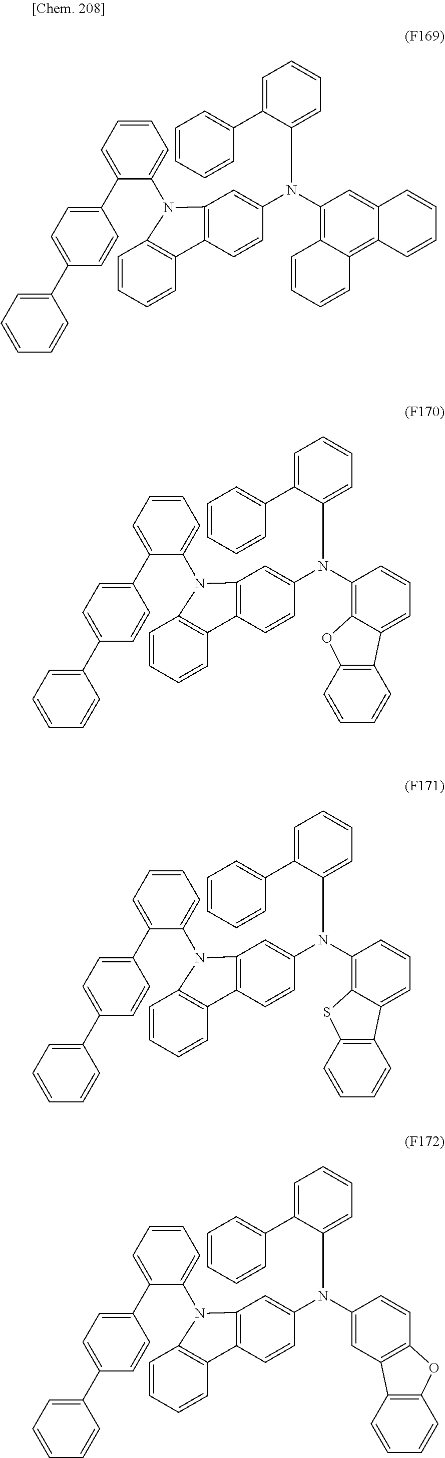 [SMILES: c1ccc(-c2ccc(-c3ccccc3-n3c4ccccc4c4ccc(N(c5ccccc5-c5ccccc5)c5cc6ccccc6c6ccccc56)cc43)cc2)cc1 ; c1ccc(-c2ccc(-c3ccccc3-n3c4ccccc4c4ccc(N(c5ccccc5-c5ccccc5)c5cccc6c5oc5ccccc56)cc43)cc2)cc1 ; c1ccc(-c2ccc(-c3ccccc3-n3c4ccccc4c4ccc(N(c5ccccc5-c5ccccc5)c5cccc6c5sc5ccccc56)cc43)cc2)cc1 ; c1ccc(-c2ccc(-c3ccccc3-n3c4ccccc4c4ccc(N(c5ccc6oc7ccccc7c6c5)c5ccccc5-c5ccccc5)cc43)cc2)cc1]

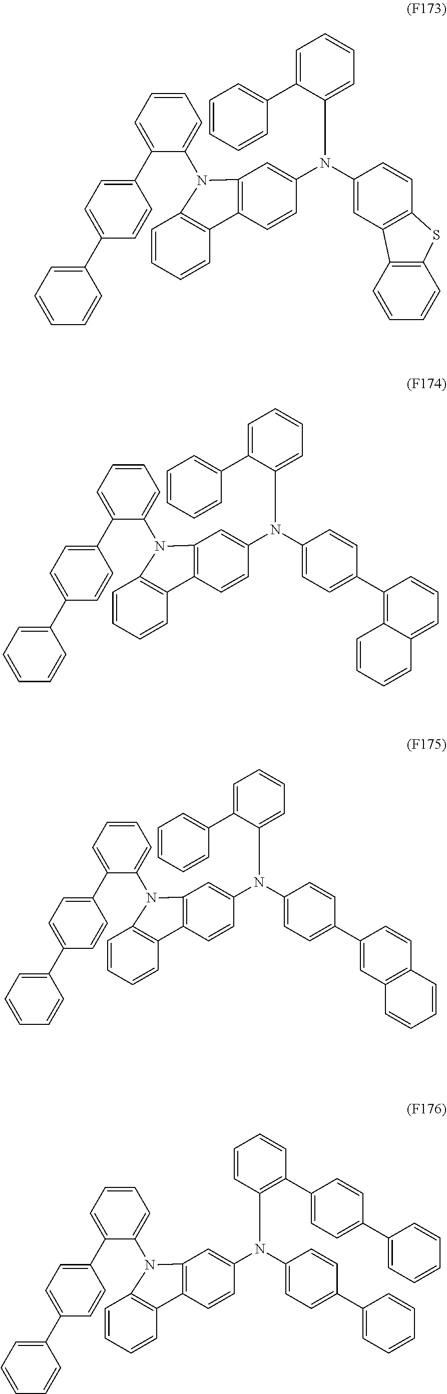 [SMILES: c1ccc(-c2ccc(-c3ccccc3-n3c4ccccc4c4ccc(N(c5ccc6sc7ccccc7c6c5)c5ccccc5-c5ccccc5)cc43)cc2)cc1 ; c1ccc(-c2ccc(-c3ccccc3-n3c4ccccc4c4ccc(N(c5ccc(-c6cccc7ccccc67)cc5)c5ccccc5-c5ccccc5)cc43)cc2)cc1 ; c1ccc(-c2ccc(-c3ccccc3-n3c4ccccc4c4ccc(N(c5ccc(-c6ccc7ccccc7c6)cc5)c5ccccc5-c5ccccc5)cc43)cc2)cc1 ; c1ccc(-c2ccc(-c3ccccc3N(c3ccc(-c4ccccc4)cc3)c3ccc4c5ccccc5n(-c5ccccc5-c5ccc(-c6ccccc6)cc5)c4c3)cc2)cc1]

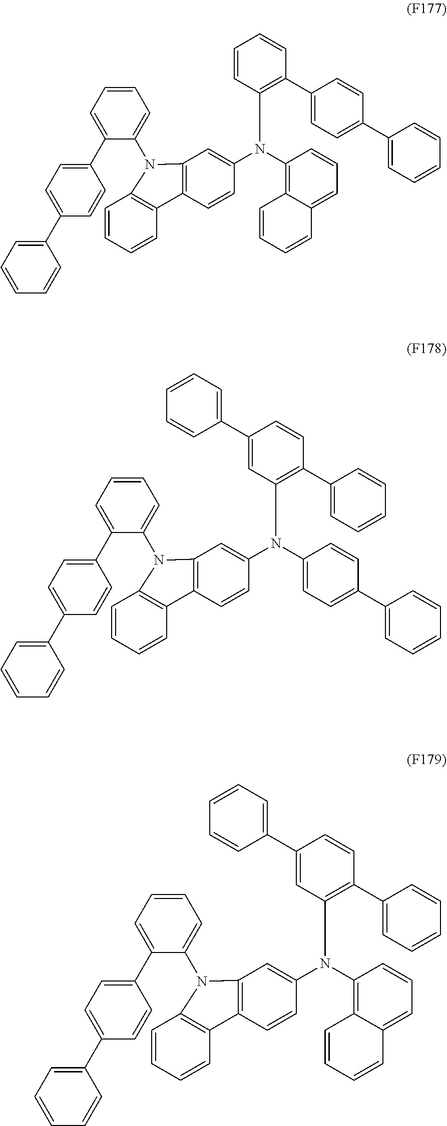 [SMILES: c1ccc(-c2ccc(-c3ccccc3N(c3ccc4c5ccccc5n(-c5ccccc5-c5ccc(-c6ccccc6)cc5)c4c3)c3cccc4ccccc34)cc2)cc1 ; c1ccc(-c2ccc(-c3ccccc3-n3c4ccccc4c4ccc(N(c5ccc(-c6ccccc6)cc5)c5cc(-c6ccccc6)ccc5-c5ccccc5)cc43)cc2)cc1 ; c1ccc(-c2ccc(-c3ccccc3-n3c4ccccc4c4ccc(N(c5cc(-c6ccccc6)ccc5-c5ccccc5)c5cccc6ccccc56)cc43)cc2)cc1]

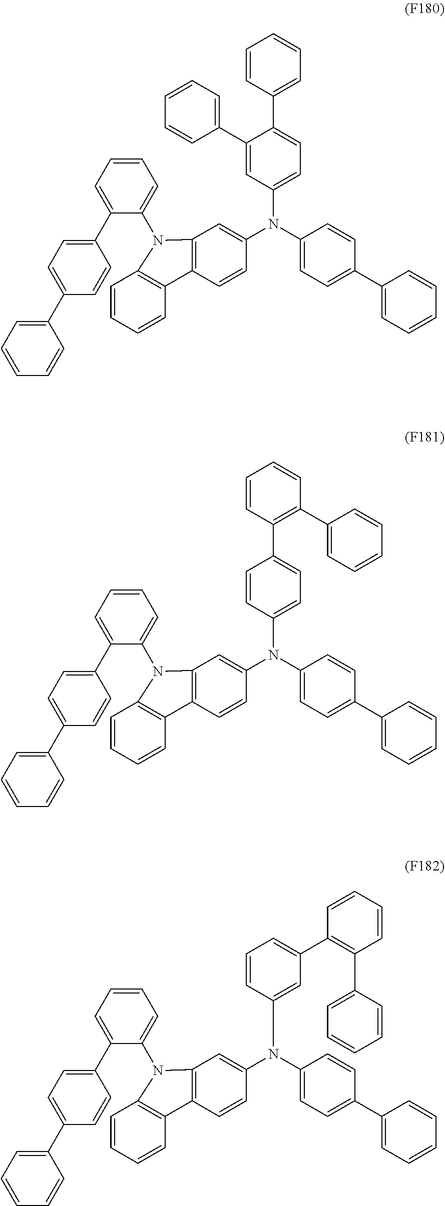 [SMILES: c1ccc(-c2ccc(-c3ccccc3-n3c4ccccc4c4ccc(N(c5ccc(-c6ccccc6)cc5)c5ccc(-c6ccccc6)c(-c6ccccc6)c5)cc43)cc2)cc1 ; c1ccc(-c2ccc(-c3ccccc3-n3c4ccccc4c4ccc(N(c5ccc(-c6ccccc6)cc5)c5ccc(-c6ccccc6-c6ccccc6)cc5)cc43)cc2)cc1 ; c1ccc(-c2ccc(-c3ccccc3-n3c4ccccc4c4ccc(N(c5ccc(-c6ccccc6)cc5)c5cccc(-c6ccccc6-c6ccccc6)c5)cc43)cc2)cc1]

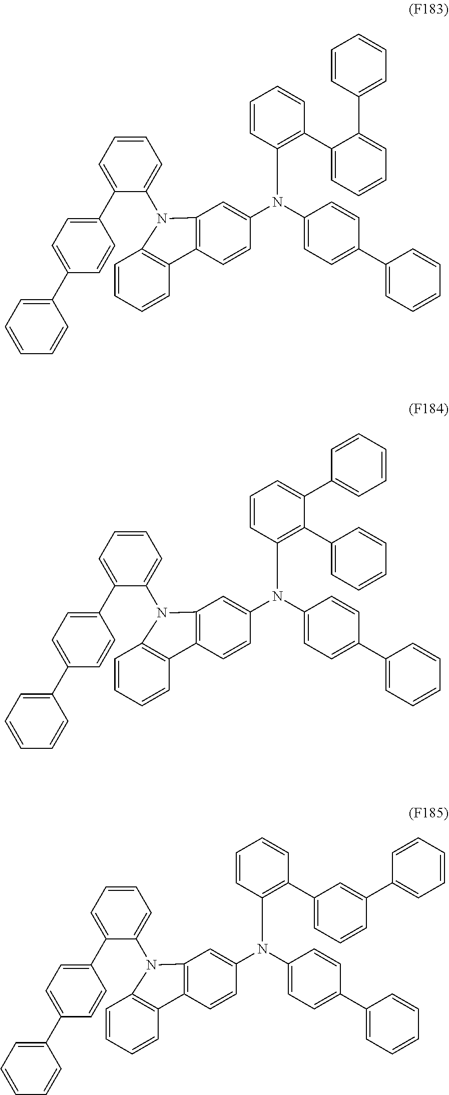 [SMILES: c1ccc(-c2ccc(-c3ccccc3-n3c4ccccc4c4ccc(N(c5ccc(-c6ccccc6)cc5)c5ccccc5-c5ccccc5-c5ccccc5)cc43)cc2)cc1 ; c1ccc(-c2ccc(-c3ccccc3-n3c4ccccc4c4ccc(N(c5ccc(-c6ccccc6)cc5)c5cccc(-c6ccccc6)c5-c5ccccc5)cc43)cc2)cc1 ; c1ccc(-c2ccc(-c3ccccc3-n3c4ccccc4c4ccc(N(c5ccc(-c6ccccc6)cc5)c5ccccc5-c5cccc(-c6ccccc6)c5)cc43)cc2)cc1]

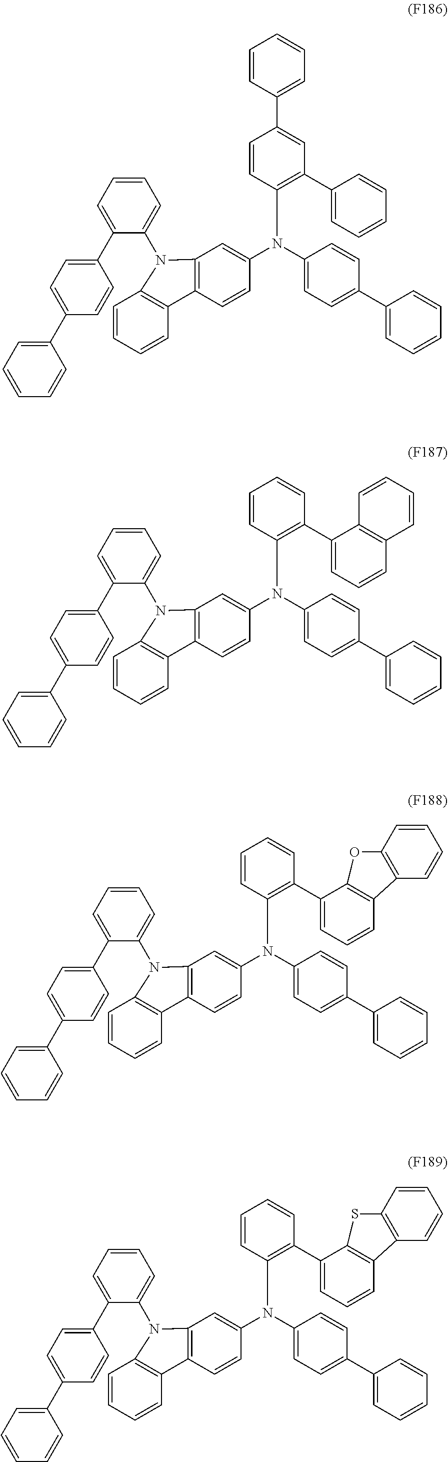 [SMILES: c1ccc(-c2ccc(-c3ccccc3-n3c4ccccc4c4ccc(N(c5ccc(-c6ccccc6)cc5)c5ccc(-c6ccccc6)cc5-c5ccccc5)cc43)cc2)cc1 ; c1ccc(-c2ccc(-c3ccccc3-n3c4ccccc4c4ccc(N(c5ccc(-c6ccccc6)cc5)c5ccccc5-c5cccc6ccccc56)cc43)cc2)cc1 ; c1ccc(-c2ccc(-c3ccccc3-n3c4ccccc4c4ccc(N(c5ccc(-c6ccccc6)cc5)c5ccccc5-c5cccc6c5oc5ccccc56)cc43)cc2)cc1 ; c1ccc(-c2ccc(-c3ccccc3-n3c4ccccc4c4ccc(N(c5ccc(-c6ccccc6)cc5)c5ccccc5-c5cccc6c5sc5ccccc56)cc43)cc2)cc1]

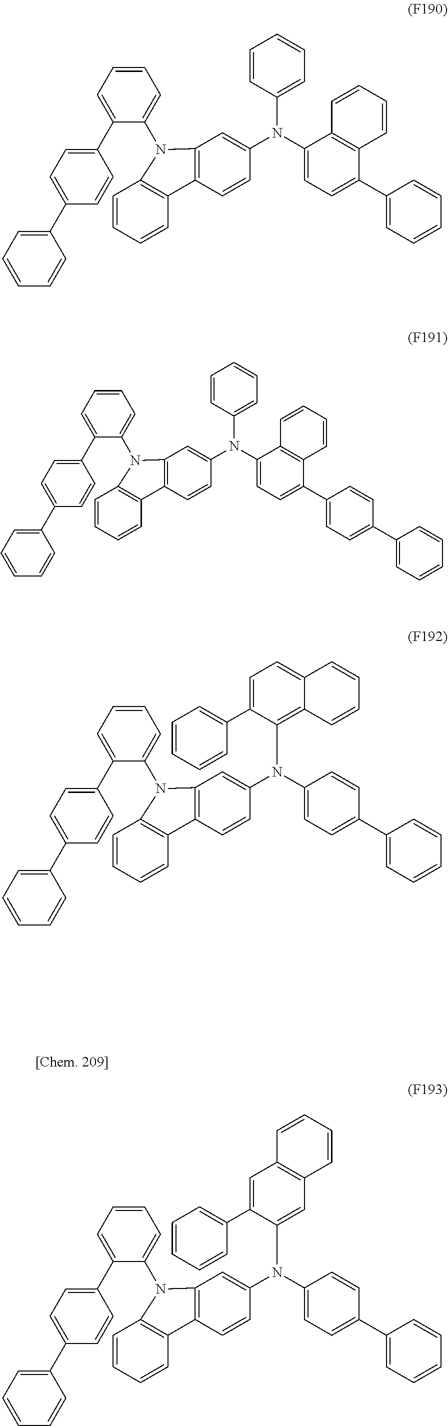 [SMILES: c1ccc(-c2ccc(-c3ccccc3-n3c4ccccc4c4ccc(N(c5ccccc5)c5ccc(-c6ccccc6)c6ccccc56)cc43)cc2)cc1 ; c1ccc(-c2ccc(-c3ccccc3-n3c4ccccc4c4ccc(N(c5ccccc5)c5ccc(-c6ccc(-c7ccccc7)cc6)c6ccccc56)cc43)cc2)cc1 ; c1ccc(-c2ccc(-c3ccccc3-n3c4ccccc4c4ccc(N(c5ccc(-c6ccccc6)cc5)c5c(-c6ccccc6)ccc6ccccc56)cc43)cc2)cc1 ; c1ccc(-c2ccc(-c3ccccc3-n3c4ccccc4c4ccc(N(c5ccc(-c6ccccc6)cc5)c5cc6ccccc6cc5-c5ccccc5)cc43)cc2)cc1]

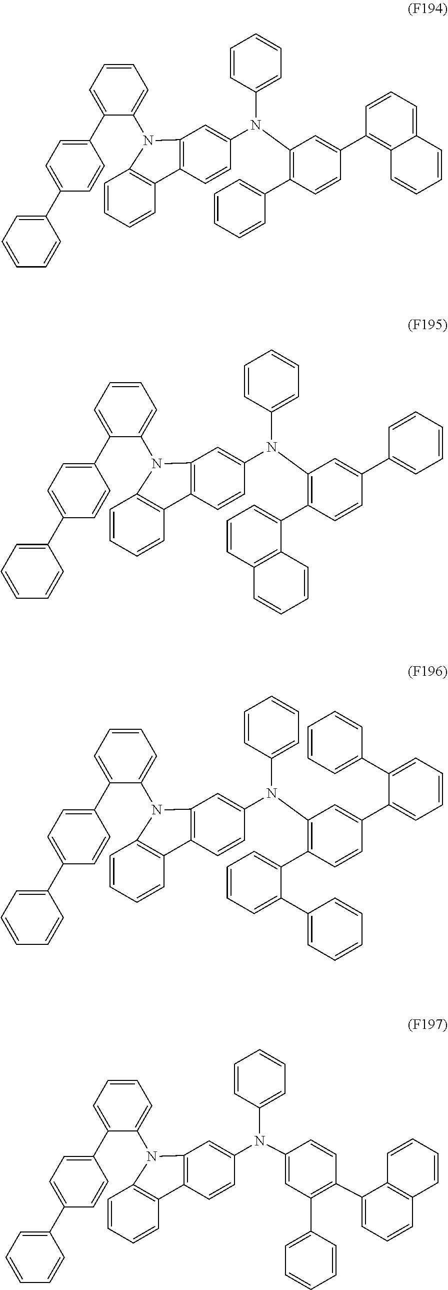 [SMILES: c1ccc(-c2ccc(-c3ccccc3-n3c4ccccc4c4ccc(N(c5ccccc5)c5cc(-c6cccc7ccccc67)ccc5-c5ccccc5)cc43)cc2)cc1 ; c1ccc(-c2ccc(-c3ccccc3-n3c4ccccc4c4ccc(N(c5ccccc5)c5cc(-c6ccccc6)ccc5-c5cccc6ccccc56)cc43)cc2)cc1 ; c1ccc(-c2ccc(-c3ccccc3-n3c4ccccc4c4ccc(N(c5ccccc5)c5cc(-c6ccccc6-c6ccccc6)ccc5-c5ccccc5-c5ccccc5)cc43)cc2)cc1 ; c1ccc(-c2ccc(-c3ccccc3-n3c4ccccc4c4ccc(N(c5ccccc5)c5ccc(-c6cccc7ccccc67)c(-c6ccccc6)c5)cc43)cc2)cc1]

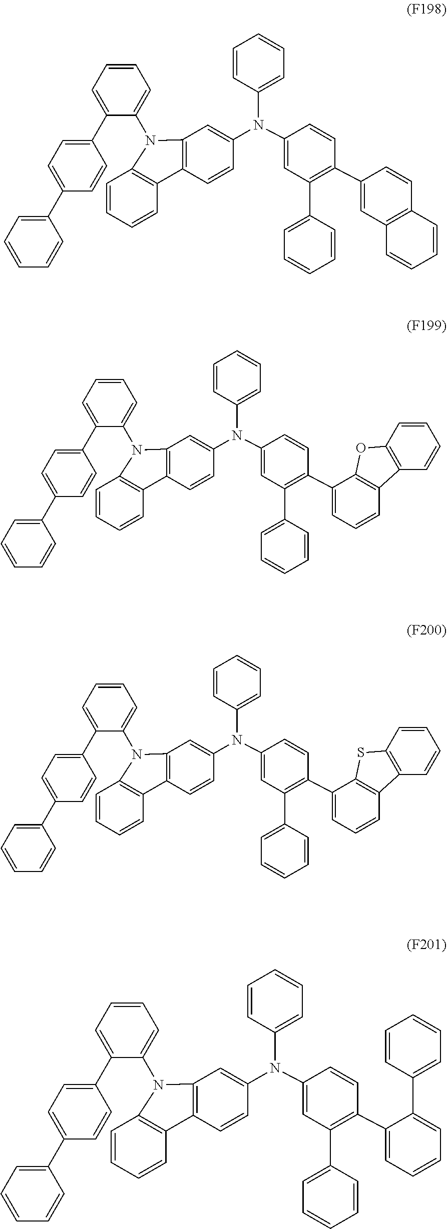 [SMILES: c1ccc(-c2ccc(-c3ccccc3-n3c4ccccc4c4ccc(N(c5ccccc5)c5ccc(-c6ccc7ccccc7c6)c(-c6ccccc6)c5)cc43)cc2)cc1 ; c1ccc(-c2ccc(-c3ccccc3-n3c4ccccc4c4ccc(N(c5ccccc5)c5ccc(-c6cccc7c6oc6ccccc67)c(-c6ccccc6)c5)cc43)cc2)cc1 ; c1ccc(-c2ccc(-c3ccccc3-n3c4ccccc4c4ccc(N(c5ccccc5)c5ccc(-c6cccc7c6sc6ccccc67)c(-c6ccccc6)c5)cc43)cc2)cc1 ; c1ccc(-c2ccc(-c3ccccc3-n3c4ccccc4c4ccc(N(c5ccccc5)c5ccc(-c6ccccc6-c6ccccc6)c(-c6ccccc6)c5)cc43)cc2)cc1]

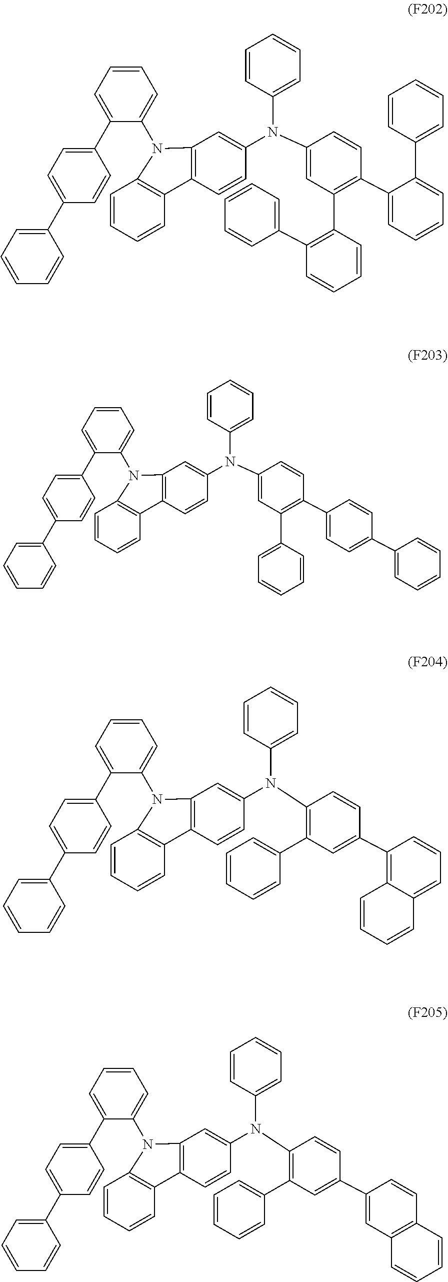 [SMILES: c1ccc(-c2ccc(-c3ccccc3-n3c4ccccc4c4ccc(N(c5ccccc5)c5ccc(-c6ccccc6-c6ccccc6)c(-c6ccccc6-c6ccccc6)c5)cc43)cc2)cc1 ; c1ccc(-c2ccc(-c3ccc(N(c4ccccc4)c4ccc5c6ccccc6n(-c6ccccc6-c6ccc(-c7ccccc7)cc6)c5c4)cc3-c3ccccc3)cc2)cc1 ; c1ccc(-c2ccc(-c3ccccc3-n3c4ccccc4c4ccc(N(c5ccccc5)c5ccc(-c6cccc7ccccc67)cc5-c5ccccc5)cc43)cc2)cc1 ; c1ccc(-c2ccc(-c3ccccc3-n3c4ccccc4c4ccc(N(c5ccccc5)c5ccc(-c6ccc7ccccc7c6)cc5-c5ccccc5)cc43)cc2)cc1]

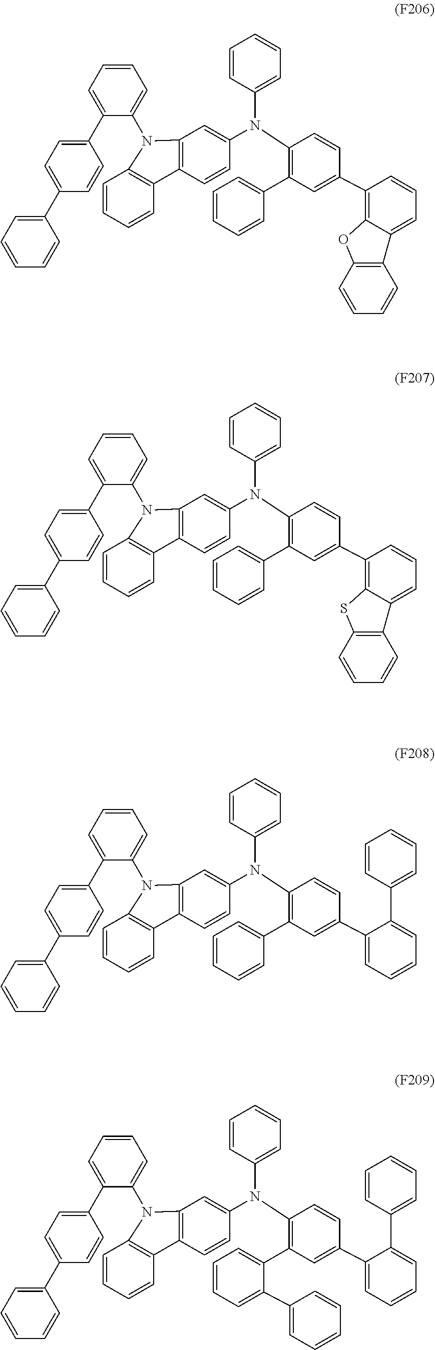 [SMILES: c1ccc(-c2ccc(-c3ccccc3-n3c4ccccc4c4ccc(N(c5ccccc5)c5ccc(-c6cccc7c6oc6ccccc67)cc5-c5ccccc5)cc43)cc2)cc1 ; c1ccc(-c2ccc(-c3ccccc3-n3c4ccccc4c4ccc(N(c5ccccc5)c5ccc(-c6cccc7c6sc6ccccc67)cc5-c5ccccc5)cc43)cc2)cc1 ; c1ccc(-c2ccc(-c3ccccc3-n3c4ccccc4c4ccc(N(c5ccccc5)c5ccc(-c6ccccc6-c6ccccc6)cc5-c5ccccc5)cc43)cc2)cc1 ; c1ccc(-c2ccc(-c3ccccc3-n3c4ccccc4c4ccc(N(c5ccccc5)c5ccc(-c6ccccc6-c6ccccc6)cc5-c5ccccc5-c5ccccc5)cc43)cc2)cc1]

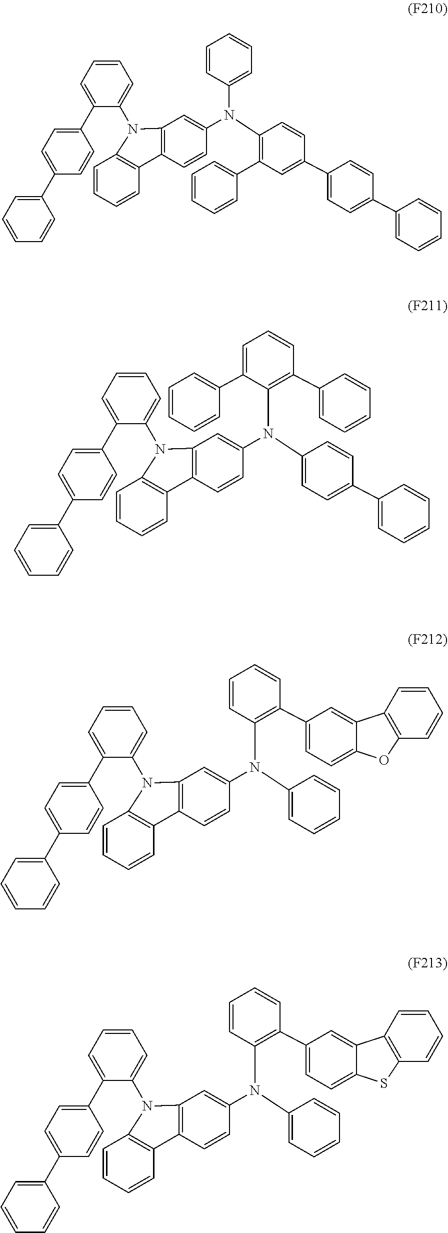 [SMILES: c1ccc(-c2ccc(-c3ccc(N(c4ccccc4)c4ccc5c6ccccc6n(-c6ccccc6-c6ccc(-c7ccccc7)cc6)c5c4)c(-c4ccccc4)c3)cc2)cc1 ; c1ccc(-c2ccc(-c3ccccc3-n3c4ccccc4c4ccc(N(c5ccc(-c6ccccc6)cc5)c5c(-c6ccccc6)cccc5-c5ccccc5)cc43)cc2)cc1 ; c1ccc(-c2ccc(-c3ccccc3-n3c4ccccc4c4ccc(N(c5ccccc5)c5ccccc5-c5ccc6oc7ccccc7c6c5)cc43)cc2)cc1 ; c1ccc(-c2ccc(-c3ccccc3-n3c4ccccc4c4ccc(N(c5ccccc5)c5ccccc5-c5ccc6sc7ccccc7c6c5)cc43)cc2)cc1]

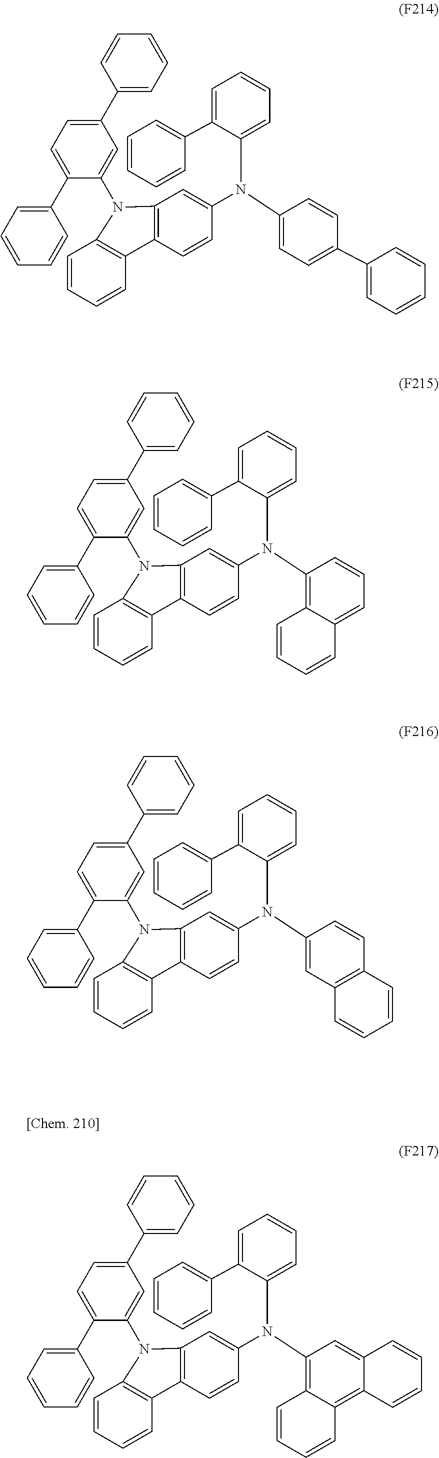 [SMILES: c1ccc(-c2ccc(N(c3ccc4c5ccccc5n(-c5cc(-c6ccccc6)ccc5-c5ccccc5)c4c3)c3ccccc3-c3ccccc3)cc2)cc1 ; c1ccc(-c2ccc(-c3ccccc3)c(-n3c4ccccc4c4ccc(N(c5ccccc5-c5ccccc5)c5cccc6ccccc56)cc43)c2)cc1 ; c1ccc(-c2ccc(-c3ccccc3)c(-n3c4ccccc4c4ccc(N(c5ccc6ccccc6c5)c5ccccc5-c5ccccc5)cc43)c2)cc1 ; c1ccc(-c2ccc(-c3ccccc3)c(-n3c4ccccc4c4ccc(N(c5ccccc5-c5ccccc5)c5cc6ccccc6c6ccccc56)cc43)c2)cc1]

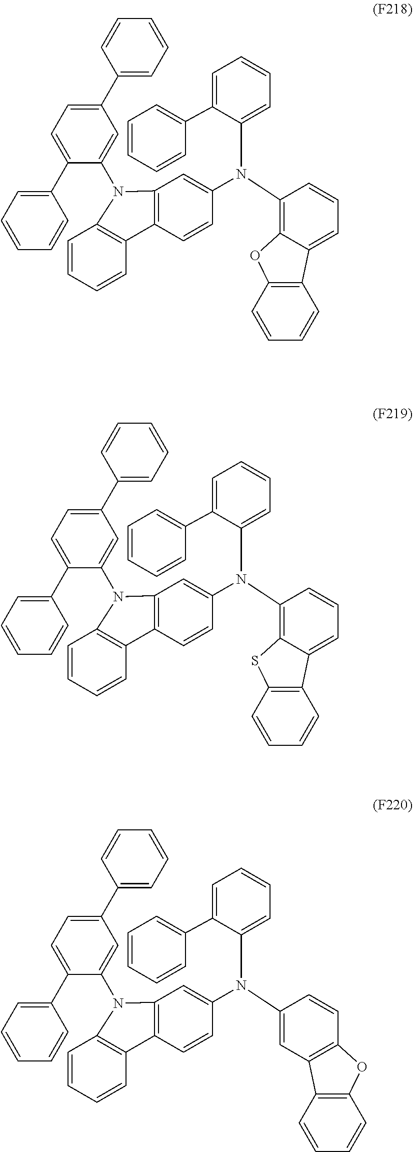 [SMILES: c1ccc(-c2ccc(-c3ccccc3)c(-n3c4ccccc4c4ccc(N(c5ccccc5-c5ccccc5)c5cccc6c5oc5ccccc56)cc43)c2)cc1 ; c1ccc(-c2ccc(-c3ccccc3)c(-n3c4ccccc4c4ccc(N(c5ccccc5-c5ccccc5)c5cccc6c5sc5ccccc56)cc43)c2)cc1 ; c1ccc(-c2ccc(-c3ccccc3)c(-n3c4ccccc4c4ccc(N(c5ccc6oc7ccccc7c6c5)c5ccccc5-c5ccccc5)cc43)c2)cc1]

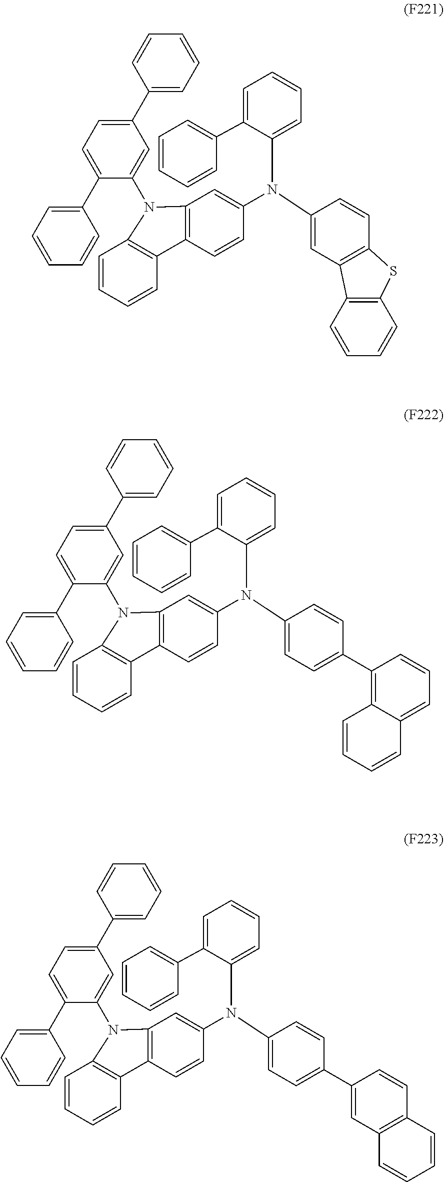 [SMILES: c1ccc(-c2ccc(-c3ccccc3)c(-n3c4ccccc4c4ccc(N(c5ccc6sc7ccccc7c6c5)c5ccccc5-c5ccccc5)cc43)c2)cc1 ; c1ccc(-c2ccc(-c3ccccc3)c(-n3c4ccccc4c4ccc(N(c5ccc(-c6cccc7ccccc67)cc5)c5ccccc5-c5ccccc5)cc43)c2)cc1 ; c1ccc(-c2ccc(-c3ccccc3)c(-n3c4ccccc4c4ccc(N(c5ccc(-c6ccc7ccccc7c6)cc5)c5ccccc5-c5ccccc5)cc43)c2)cc1]

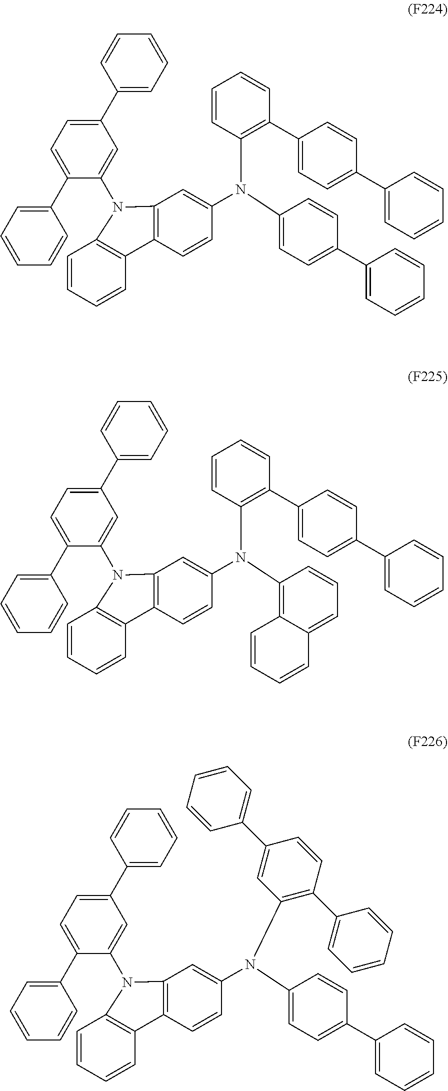 [SMILES: c1ccc(-c2ccc(-c3ccccc3N(c3ccc(-c4ccccc4)cc3)c3ccc4c5ccccc5n(-c5cc(-c6ccccc6)ccc5-c5ccccc5)c4c3)cc2)cc1 ; c1ccc(-c2ccc(-c3ccccc3N(c3ccc4c5ccccc5n(-c5cc(-c6ccccc6)ccc5-c5ccccc5)c4c3)c3cccc4ccccc34)cc2)cc1 ; c1ccc(-c2ccc(N(c3ccc4c5ccccc5n(-c5cc(-c6ccccc6)ccc5-c5ccccc5)c4c3)c3cc(-c4ccccc4)ccc3-c3ccccc3)cc2)cc1]

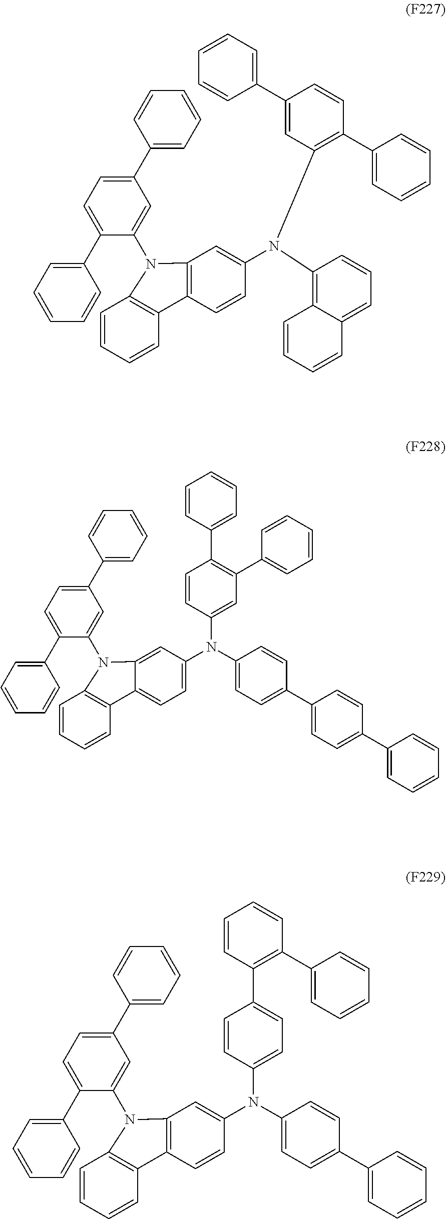 [SMILES: c1ccc(-c2ccc(-c3ccccc3)c(N(c3ccc4c5ccccc5n(-c5cc(-c6ccccc6)ccc5-c5ccccc5)c4c3)c3cccc4ccccc34)c2)cc1 ; c1ccc(-c2ccc(-c3ccc(N(c4ccc(-c5ccccc5)c(-c5ccccc5)c4)c4ccc5c6ccccc6n(-c6cc(-c7ccccc7)ccc6-c6ccccc6)c5c4)cc3)cc2)cc1 ; c1ccc(-c2ccc(N(c3ccc(-c4ccccc4-c4ccccc4)cc3)c3ccc4c5ccccc5n(-c5cc(-c6ccccc6)ccc5-c5ccccc5)c4c3)cc2)cc1]

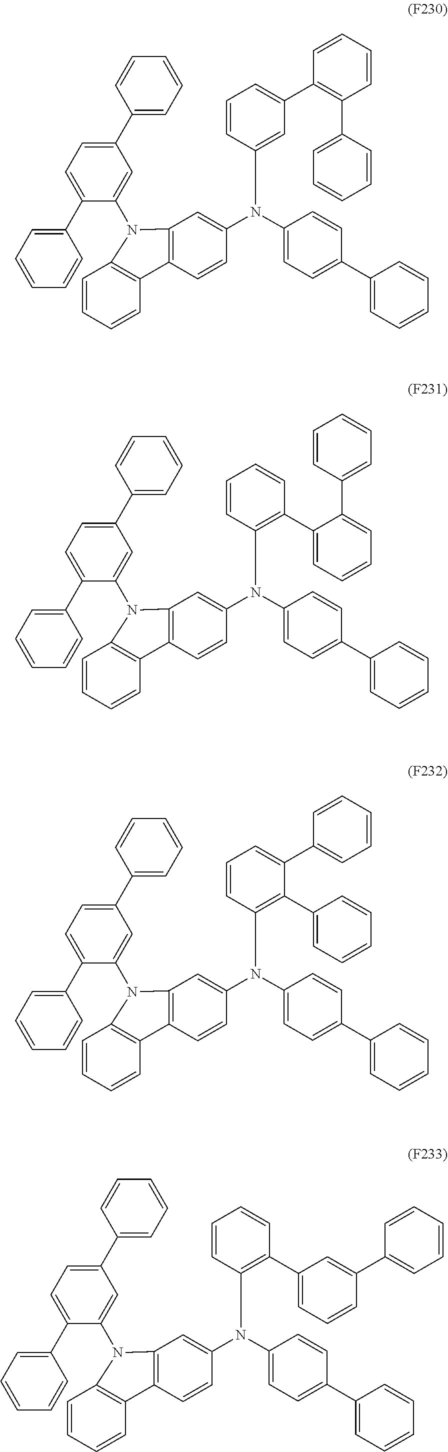 [SMILES: c1ccc(-c2ccc(N(c3cccc(-c4ccccc4-c4ccccc4)c3)c3ccc4c5ccccc5n(-c5cc(-c6ccccc6)ccc5-c5ccccc5)c4c3)cc2)cc1 ; c1ccc(-c2ccc(N(c3ccc4c5ccccc5n(-c5cc(-c6ccccc6)ccc5-c5ccccc5)c4c3)c3ccccc3-c3ccccc3-c3ccccc3)cc2)cc1 ; c1ccc(-c2ccc(N(c3ccc4c5ccccc5n(-c5cc(-c6ccccc6)ccc5-c5ccccc5)c4c3)c3cccc(-c4ccccc4)c3-c3ccccc3)cc2)cc1 ; c1ccc(-c2ccc(N(c3ccc4c5ccccc5n(-c5cc(-c6ccccc6)ccc5-c5ccccc5)c4c3)c3ccccc3-c3cccc(-c4ccccc4)c3)cc2)cc1]

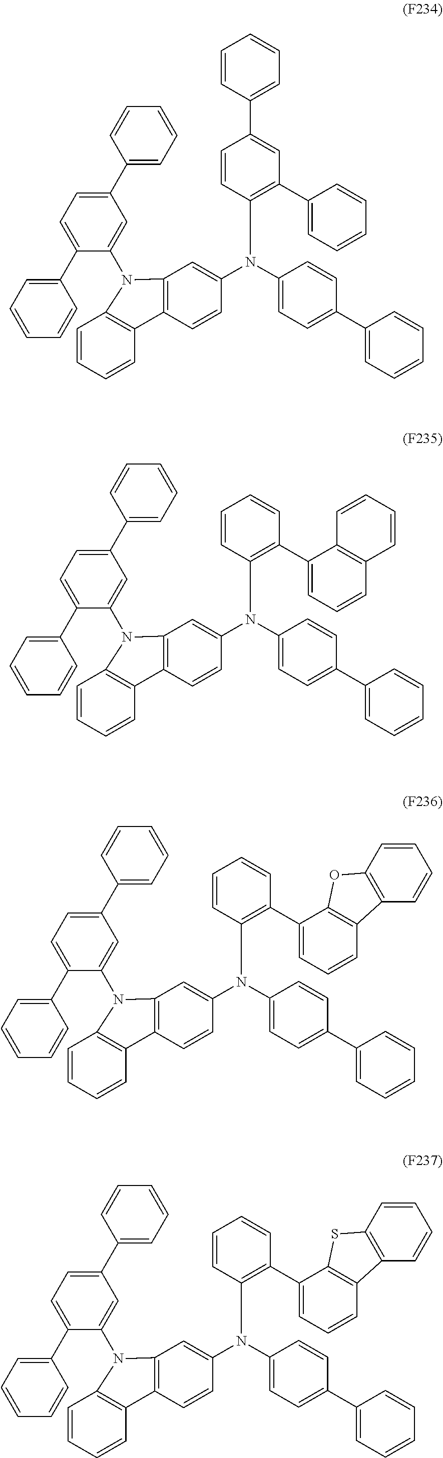 [SMILES: c1ccc(-c2ccc(N(c3ccc4c5ccccc5n(-c5cc(-c6ccccc6)ccc5-c5ccccc5)c4c3)c3ccc(-c4ccccc4)cc3-c3ccccc3)cc2)cc1 ; c1ccc(-c2ccc(N(c3ccc4c5ccccc5n(-c5cc(-c6ccccc6)ccc5-c5ccccc5)c4c3)c3ccccc3-c3cccc4ccccc34)cc2)cc1 ; c1ccc(-c2ccc(N(c3ccc4c5ccccc5n(-c5cc(-c6ccccc6)ccc5-c5ccccc5)c4c3)c3ccccc3-c3cccc4c3oc3ccccc34)cc2)cc1 ; c1ccc(-c2ccc(N(c3ccc4c5ccccc5n(-c5cc(-c6ccccc6)ccc5-c5ccccc5)c4c3)c3ccccc3-c3cccc4c3sc3ccccc34)cc2)cc1]

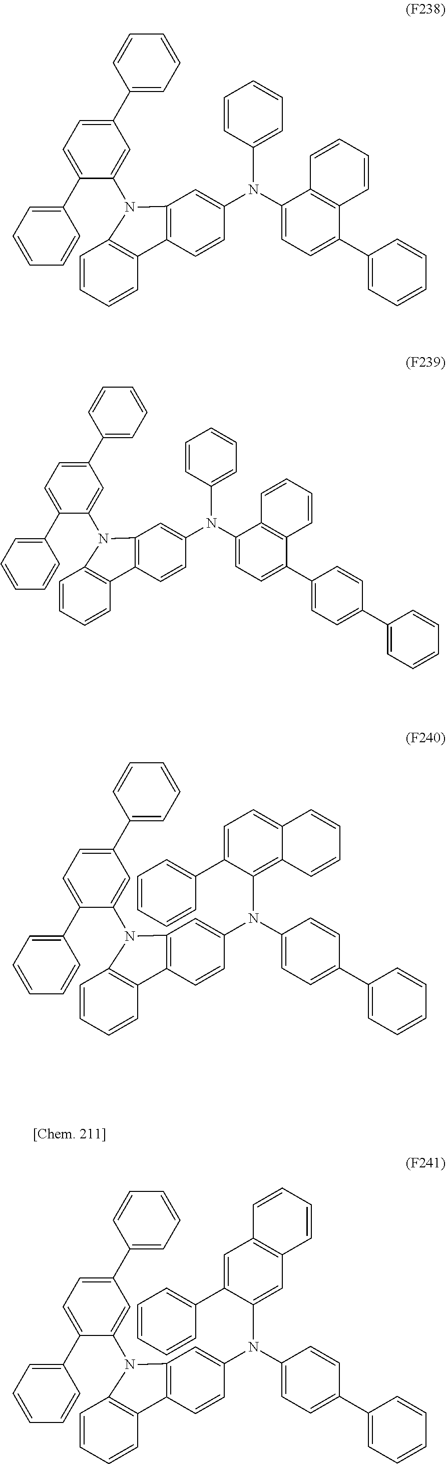 [SMILES: c1ccc(-c2ccc(-c3ccccc3)c(-n3c4ccccc4c4ccc(N(c5ccccc5)c5ccc(-c6ccccc6)c6ccccc56)cc43)c2)cc1 ; c1ccc(-c2ccc(-c3ccc(N(c4ccccc4)c4ccc5c6ccccc6n(-c6cc(-c7ccccc7)ccc6-c6ccccc6)c5c4)c4ccccc34)cc2)cc1 ; c1ccc(-c2ccc(N(c3ccc4c5ccccc5n(-c5cc(-c6ccccc6)ccc5-c5ccccc5)c4c3)c3c(-c4ccccc4)ccc4ccccc34)cc2)cc1 ; c1ccc(-c2ccc(N(c3ccc4c5ccccc5n(-c5cc(-c6ccccc6)ccc5-c5ccccc5)c4c3)c3cc4ccccc4cc3-c3ccccc3)cc2)cc1]

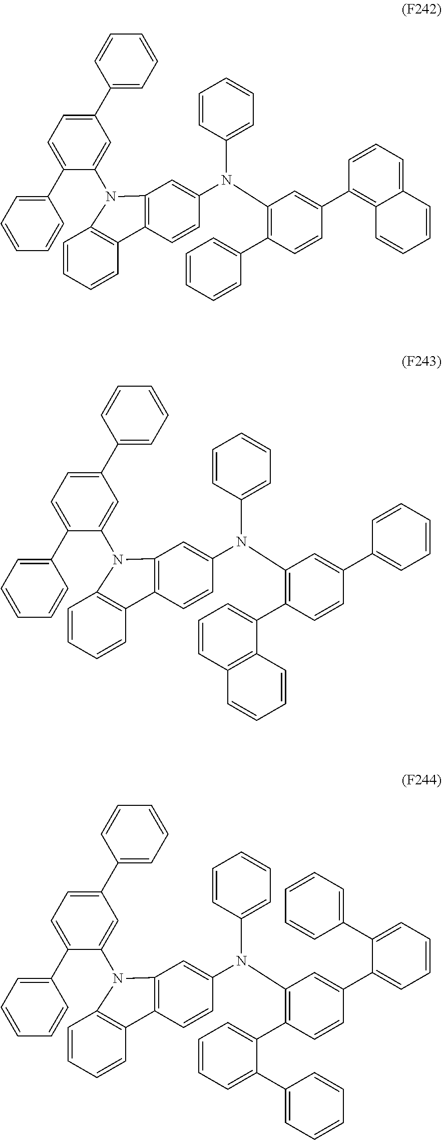 [SMILES: c1ccc(-c2ccc(-c3ccccc3)c(-n3c4ccccc4c4ccc(N(c5ccccc5)c5cc(-c6cccc7ccccc67)ccc5-c5ccccc5)cc43)c2)cc1 ; c1ccc(-c2ccc(-c3cccc4ccccc34)c(N(c3ccccc3)c3ccc4c5ccccc5n(-c5cc(-c6ccccc6)ccc5-c5ccccc5)c4c3)c2)cc1 ; c1ccc(-c2ccc(-c3ccccc3)c(-n3c4ccccc4c4ccc(N(c5ccccc5)c5cc(-c6ccccc6-c6ccccc6)ccc5-c5ccccc5-c5ccccc5)cc43)c2)cc1]

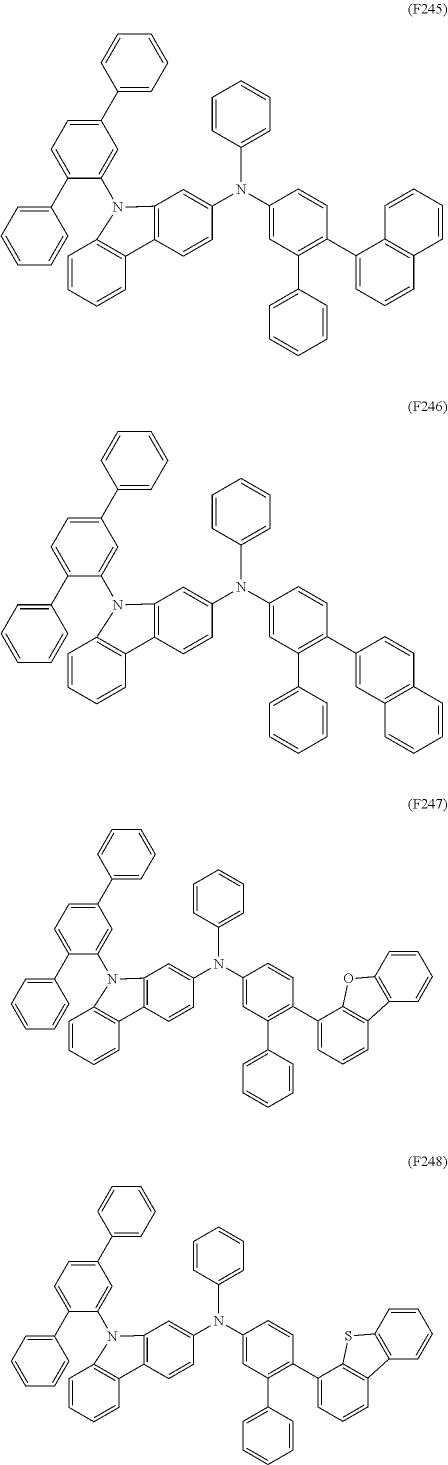 [SMILES: c1ccc(-c2ccc(-c3ccccc3)c(-n3c4ccccc4c4ccc(N(c5ccccc5)c5ccc(-c6cccc7ccccc67)c(-c6ccccc6)c5)cc43)c2)cc1 ; c1ccc(-c2ccc(-c3ccccc3)c(-n3c4ccccc4c4ccc(N(c5ccccc5)c5ccc(-c6ccc7ccccc7c6)c(-c6ccccc6)c5)cc43)c2)cc1 ; c1ccc(-c2ccc(-c3ccccc3)c(-n3c4ccccc4c4ccc(N(c5ccccc5)c5ccc(-c6cccc7c6oc6ccccc67)c(-c6ccccc6)c5)cc43)c2)cc1 ; c1ccc(-c2ccc(-c3ccccc3)c(-n3c4ccccc4c4ccc(N(c5ccccc5)c5ccc(-c6cccc7c6sc6ccccc67)c(-c6ccccc6)c5)cc43)c2)cc1]

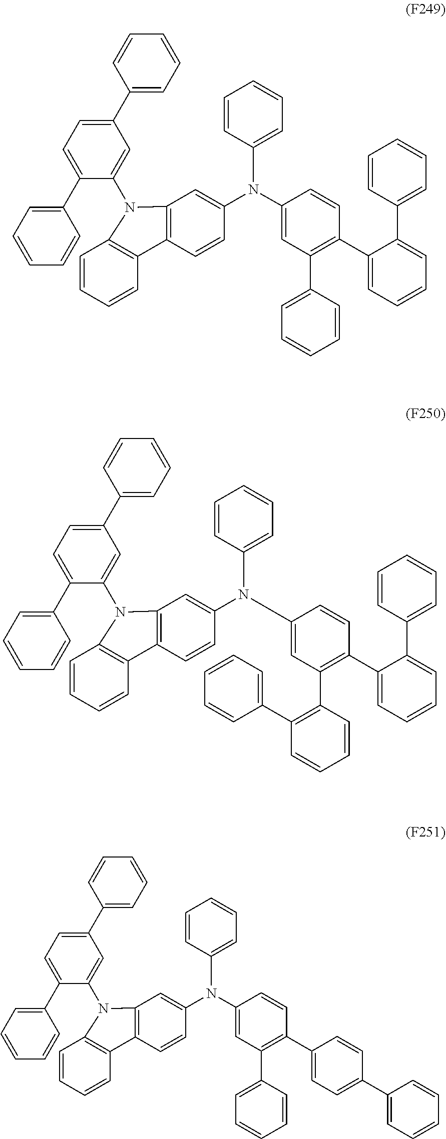 [SMILES: c1ccc(-c2ccc(-c3ccccc3)c(-n3c4ccccc4c4ccc(N(c5ccccc5)c5ccc(-c6ccccc6-c6ccccc6)c(-c6ccccc6)c5)cc43)c2)cc1 ; c1ccc(-c2ccc(-c3ccccc3)c(-n3c4ccccc4c4ccc(N(c5ccccc5)c5ccc(-c6ccccc6-c6ccccc6)c(-c6ccccc6-c6ccccc6)c5)cc43)c2)cc1 ; c1ccc(-c2ccc(-c3ccc(N(c4ccccc4)c4ccc5c6ccccc6n(-c6cc(-c7ccccc7)ccc6-c6ccccc6)c5c4)cc3-c3ccccc3)cc2)cc1]

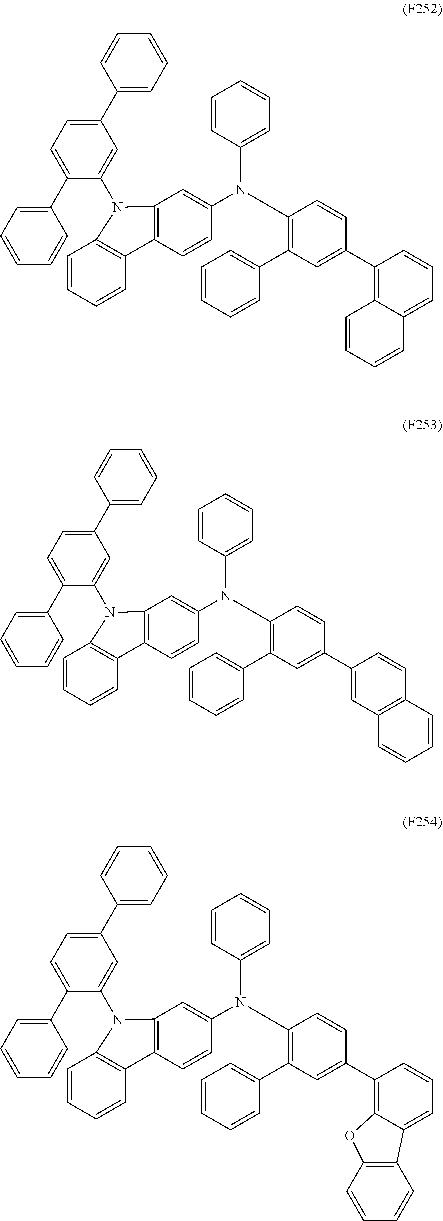 [SMILES: c1ccc(-c2ccc(-c3ccccc3)c(-n3c4ccccc4c4ccc(N(c5ccccc5)c5ccc(-c6cccc7ccccc67)cc5-c5ccccc5)cc43)c2)cc1 ; c1ccc(-c2ccc(-c3ccccc3)c(-n3c4ccccc4c4ccc(N(c5ccccc5)c5ccc(-c6ccc7ccccc7c6)cc5-c5ccccc5)cc43)c2)cc1 ; c1ccc(-c2ccc(-c3ccccc3)c(-n3c4ccccc4c4ccc(N(c5ccccc5)c5ccc(-c6cccc7c6oc6ccccc67)cc5-c5ccccc5)cc43)c2)cc1]

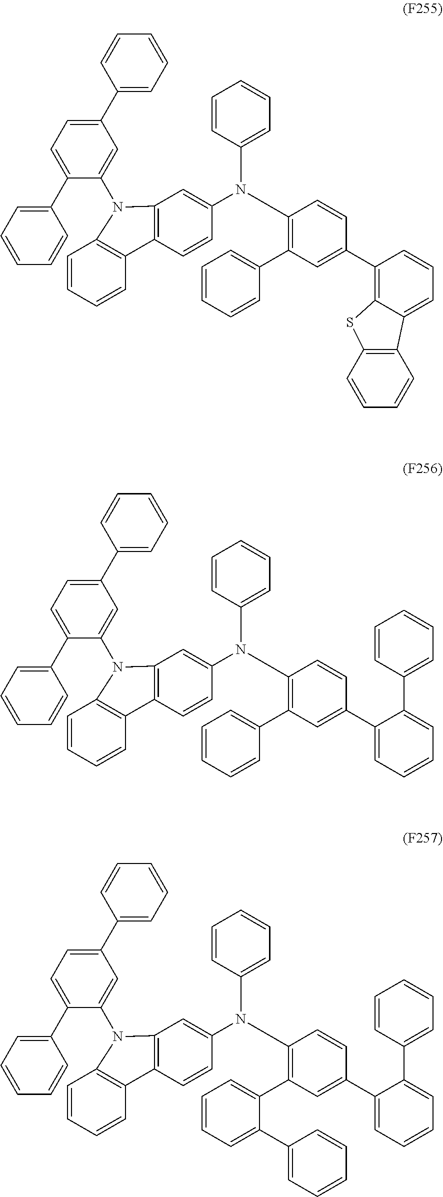 [SMILES: c1ccc(-c2ccc(-c3ccccc3)c(-n3c4ccccc4c4ccc(N(c5ccccc5)c5ccc(-c6cccc7c6sc6ccccc67)cc5-c5ccccc5)cc43)c2)cc1 ; c1ccc(-c2ccc(-c3ccccc3)c(-n3c4ccccc4c4ccc(N(c5ccccc5)c5ccc(-c6ccccc6-c6ccccc6)cc5-c5ccccc5)cc43)c2)cc1 ; c1ccc(-c2ccc(-c3ccccc3)c(-n3c4ccccc4c4ccc(N(c5ccccc5)c5ccc(-c6ccccc6-c6ccccc6)cc5-c5ccccc5-c5ccccc5)cc43)c2)cc1]

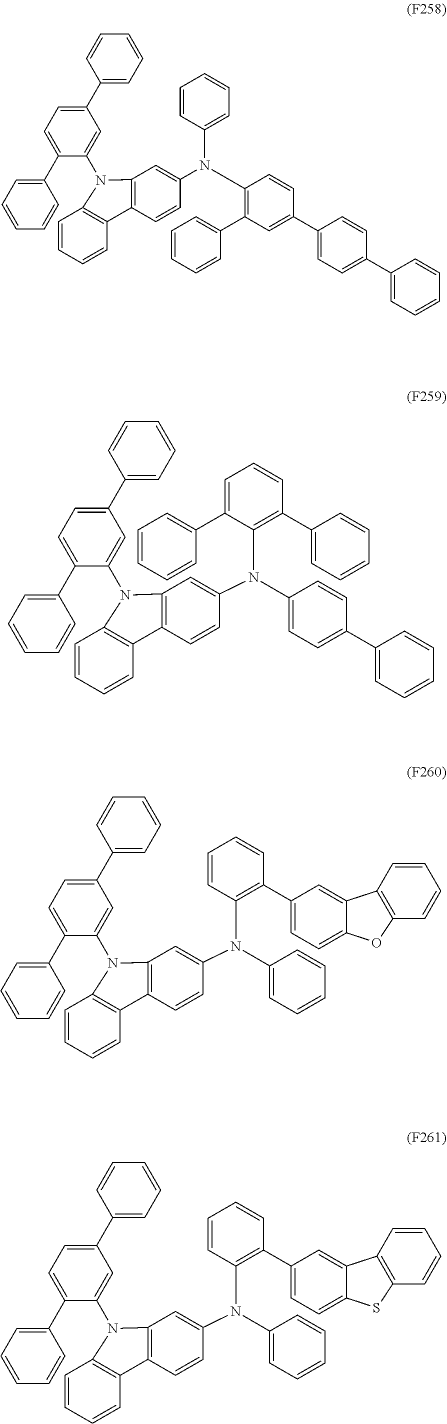 [SMILES: c1ccc(-c2ccc(-c3ccc(N(c4ccccc4)c4ccc5c6ccccc6n(-c6cc(-c7ccccc7)ccc6-c6ccccc6)c5c4)c(-c4ccccc4)c3)cc2)cc1 ; c1ccc(-c2ccc(N(c3ccc4c5ccccc5n(-c5cc(-c6ccccc6)ccc5-c5ccccc5)c4c3)c3c(-c4ccccc4)cccc3-c3ccccc3)cc2)cc1 ; c1ccc(-c2ccc(-c3ccccc3)c(-n3c4ccccc4c4ccc(N(c5ccccc5)c5ccccc5-c5ccc6oc7ccccc7c6c5)cc43)c2)cc1 ; c1ccc(-c2ccc(-c3ccccc3)c(-n3c4ccccc4c4ccc(N(c5ccccc5)c5ccccc5-c5ccc6sc7ccccc7c6c5)cc43)c2)cc1]

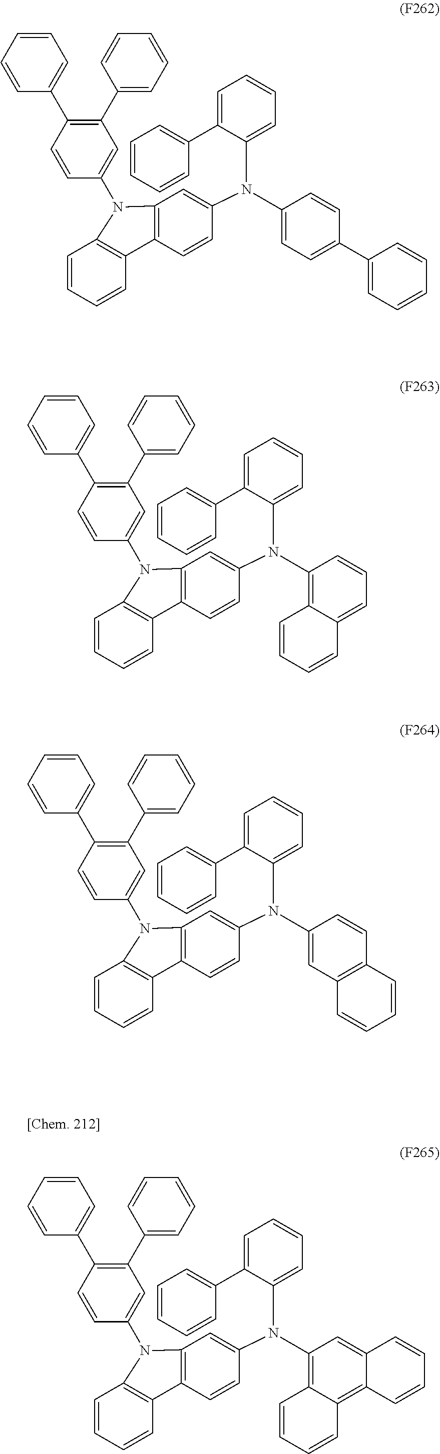 [SMILES: c1ccc(-c2ccc(N(c3ccc4c5ccccc5n(-c5ccc(-c6ccccc6)c(-c6ccccc6)c5)c4c3)c3ccccc3-c3ccccc3)cc2)cc1 ; c1ccc(-c2ccc(-n3c4ccccc4c4ccc(N(c5ccccc5-c5ccccc5)c5cccc6ccccc56)cc43)cc2-c2ccccc2)cc1 ; c1ccc(-c2ccc(-n3c4ccccc4c4ccc(N(c5ccc6ccccc6c5)c5ccccc5-c5ccccc5)cc43)cc2-c2ccccc2)cc1 ; c1ccc(-c2ccc(-n3c4ccccc4c4ccc(N(c5ccccc5-c5ccccc5)c5cc6ccccc6c6ccccc56)cc43)cc2-c2ccccc2)cc1]

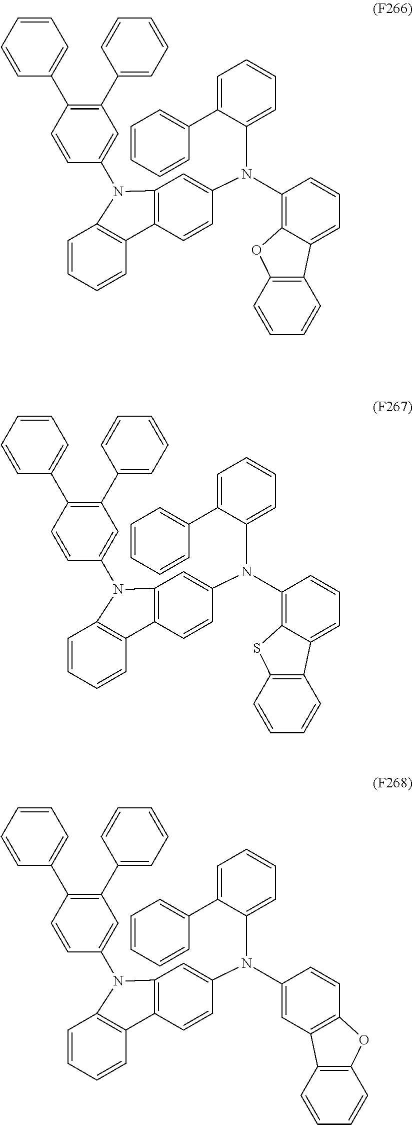 [SMILES: c1ccc(-c2ccc(-n3c4ccccc4c4ccc(N(c5ccccc5-c5ccccc5)c5cccc6c5oc5ccccc56)cc43)cc2-c2ccccc2)cc1 ; c1ccc(-c2ccc(-n3c4ccccc4c4ccc(N(c5ccccc5-c5ccccc5)c5cccc6c5sc5ccccc56)cc43)cc2-c2ccccc2)cc1 ; c1ccc(-c2ccc(-n3c4ccccc4c4ccc(N(c5ccc6oc7ccccc7c6c5)c5ccccc5-c5ccccc5)cc43)cc2-c2ccccc2)cc1]

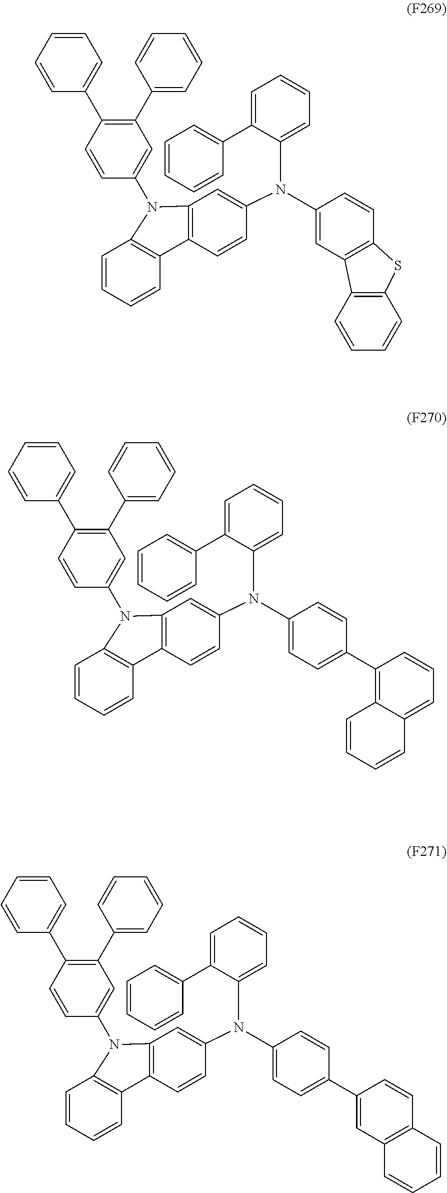 [SMILES: c1ccc(-c2ccc(-n3c4ccccc4c4ccc(N(c5ccc6sc7ccccc7c6c5)c5ccccc5-c5ccccc5)cc43)cc2-c2ccccc2)cc1 ; c1ccc(-c2ccc(-n3c4ccccc4c4ccc(N(c5ccc(-c6cccc7ccccc67)cc5)c5ccccc5-c5ccccc5)cc43)cc2-c2ccccc2)cc1 ; c1ccc(-c2ccc(-n3c4ccccc4c4ccc(N(c5ccc(-c6ccc7ccccc7c6)cc5)c5ccccc5-c5ccccc5)cc43)cc2-c2ccccc2)cc1]

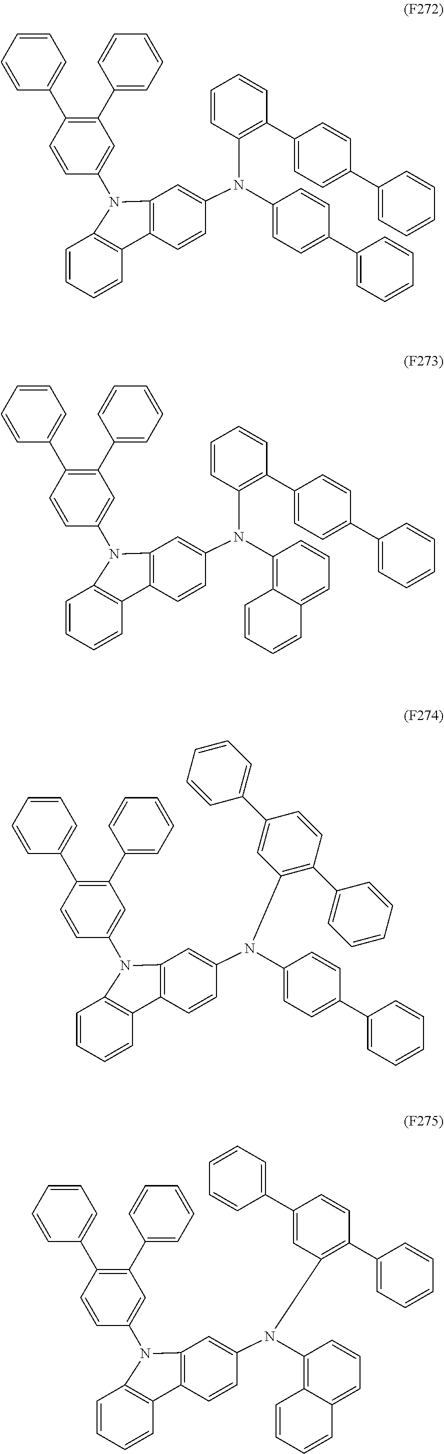 [SMILES: c1ccc(-c2ccc(-c3ccccc3N(c3ccc(-c4ccccc4)cc3)c3ccc4c5ccccc5n(-c5ccc(-c6ccccc6)c(-c6ccccc6)c5)c4c3)cc2)cc1 ; c1ccc(-c2ccc(-c3ccccc3N(c3ccc4c5ccccc5n(-c5ccc(-c6ccccc6)c(-c6ccccc6)c5)c4c3)c3cccc4ccccc34)cc2)cc1 ; c1ccc(-c2ccc(N(c3ccc4c5ccccc5n(-c5ccc(-c6ccccc6)c(-c6ccccc6)c5)c4c3)c3cc(-c4ccccc4)ccc3-c3ccccc3)cc2)cc1 ; c1ccc(-c2ccc(-c3ccccc3)c(N(c3ccc4c5ccccc5n(-c5ccc(-c6ccccc6)c(-c6ccccc6)c5)c4c3)c3cccc4ccccc34)c2)cc1]

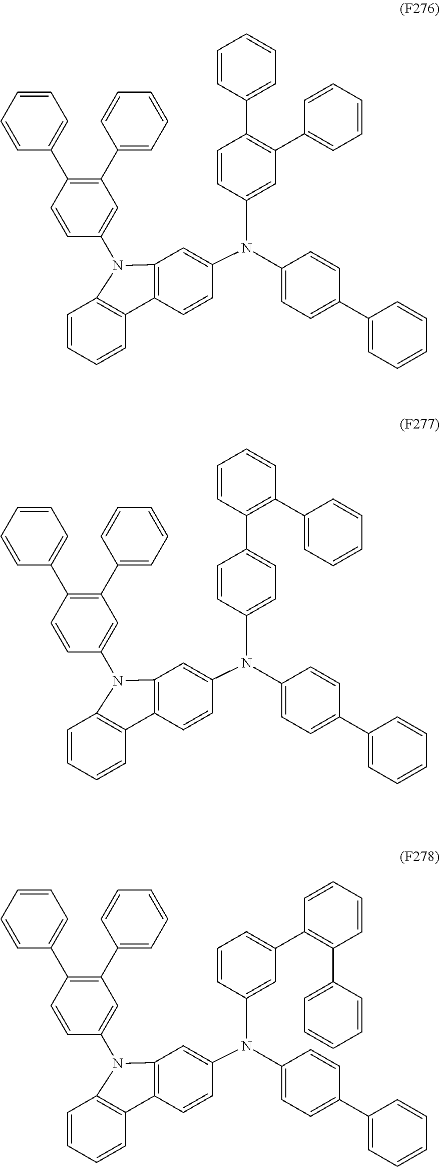 [SMILES: c1ccc(-c2ccc(N(c3ccc(-c4ccccc4)c(-c4ccccc4)c3)c3ccc4c5ccccc5n(-c5ccc(-c6ccccc6)c(-c6ccccc6)c5)c4c3)cc2)cc1 ; c1ccc(-c2ccc(N(c3ccc(-c4ccccc4-c4ccccc4)cc3)c3ccc4c5ccccc5n(-c5ccc(-c6ccccc6)c(-c6ccccc6)c5)c4c3)cc2)cc1 ; c1ccc(-c2ccc(N(c3cccc(-c4ccccc4-c4ccccc4)c3)c3ccc4c5ccccc5n(-c5ccc(-c6ccccc6)c(-c6ccccc6)c5)c4c3)cc2)cc1]

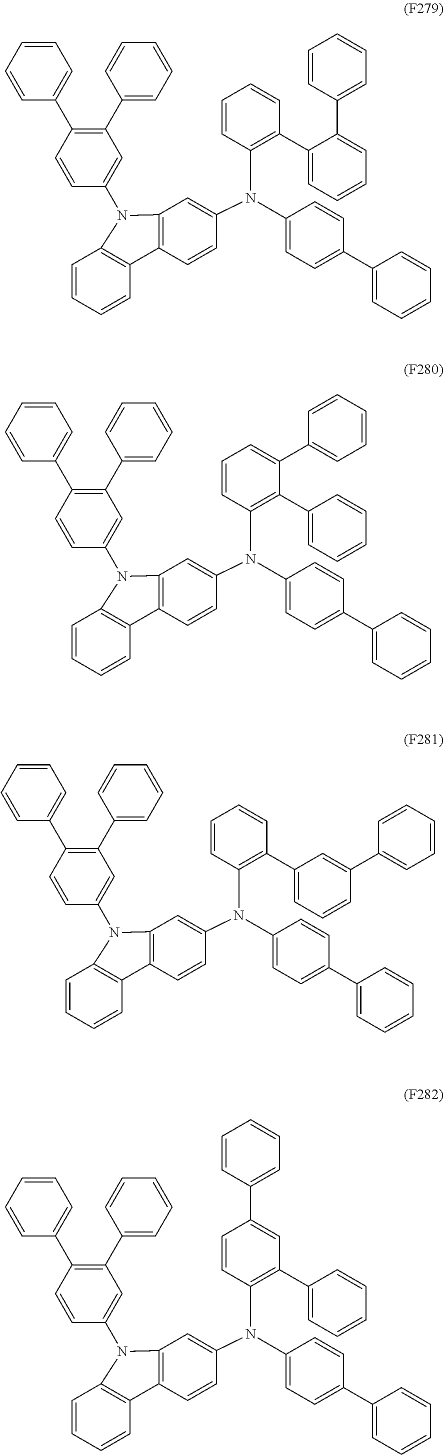 [SMILES: c1ccc(-c2ccc(N(c3ccc4c5ccccc5n(-c5ccc(-c6ccccc6)c(-c6ccccc6)c5)c4c3)c3ccccc3-c3ccccc3-c3ccccc3)cc2)cc1 ; c1ccc(-c2ccc(N(c3ccc4c5ccccc5n(-c5ccc(-c6ccccc6)c(-c6ccccc6)c5)c4c3)c3cccc(-c4ccccc4)c3-c3ccccc3)cc2)cc1 ; c1ccc(-c2ccc(N(c3ccc4c5ccccc5n(-c5ccc(-c6ccccc6)c(-c6ccccc6)c5)c4c3)c3ccccc3-c3cccc(-c4ccccc4)c3)cc2)cc1 ; c1ccc(-c2ccc(N(c3ccc4c5ccccc5n(-c5ccc(-c6ccccc6)c(-c6ccccc6)c5)c4c3)c3ccc(-c4ccccc4)cc3-c3ccccc3)cc2)cc1]

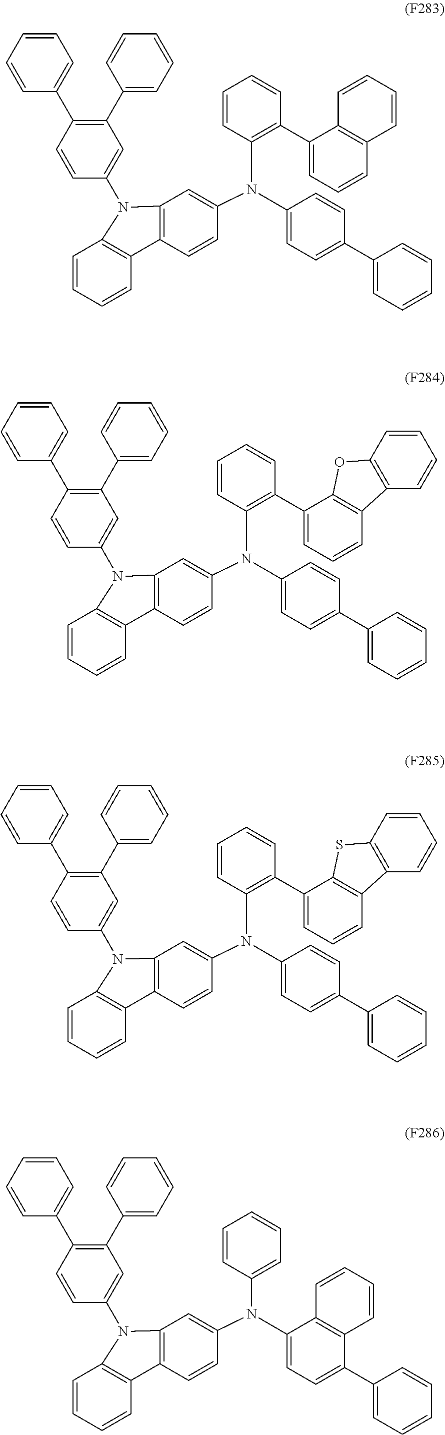 [SMILES: c1ccc(-c2ccc(N(c3ccc4c5ccccc5n(-c5ccc(-c6ccccc6)c(-c6ccccc6)c5)c4c3)c3ccccc3-c3cccc4ccccc34)cc2)cc1 ; c1ccc(-c2ccc(N(c3ccc4c5ccccc5n(-c5ccc(-c6ccccc6)c(-c6ccccc6)c5)c4c3)c3ccccc3-c3cccc4c3oc3ccccc34)cc2)cc1 ; c1ccc(-c2ccc(N(c3ccc4c5ccccc5n(-c5ccc(-c6ccccc6)c(-c6ccccc6)c5)c4c3)c3ccccc3-c3cccc4c3sc3ccccc34)cc2)cc1 ; c1ccc(-c2ccc(-n3c4ccccc4c4ccc(N(c5ccccc5)c5ccc(-c6ccccc6)c6ccccc56)cc43)cc2-c2ccccc2)cc1]

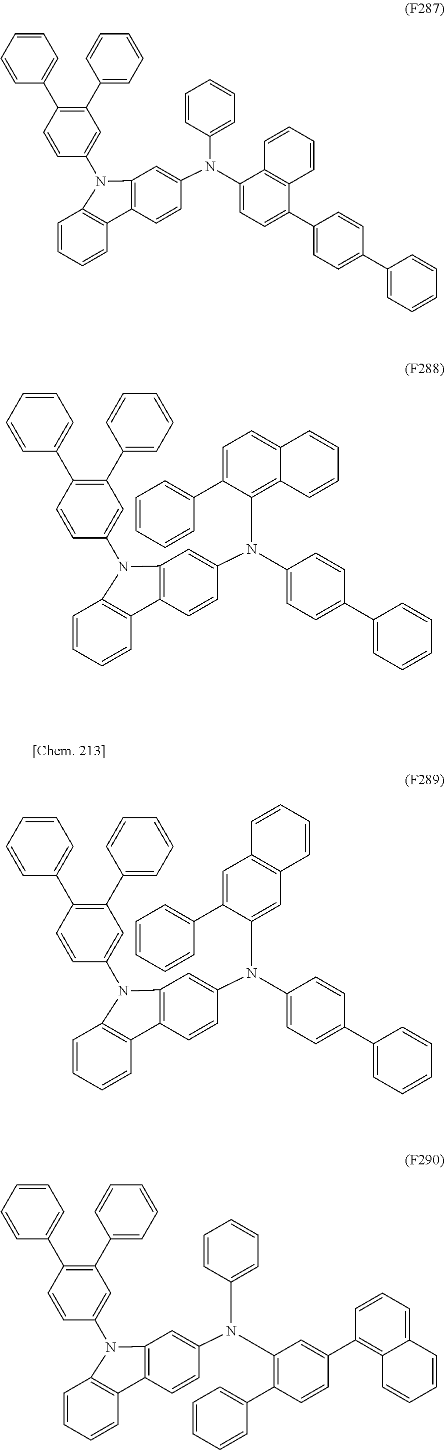 [SMILES: c1ccc(-c2ccc(-c3ccc(N(c4ccccc4)c4ccc5c6ccccc6n(-c6ccc(-c7ccccc7)c(-c7ccccc7)c6)c5c4)c4ccccc34)cc2)cc1 ; c1ccc(-c2ccc(N(c3ccc4c5ccccc5n(-c5ccc(-c6ccccc6)c(-c6ccccc6)c5)c4c3)c3c(-c4ccccc4)ccc4ccccc34)cc2)cc1 ; c1ccc(-c2ccc(N(c3ccc4c5ccccc5n(-c5ccc(-c6ccccc6)c(-c6ccccc6)c5)c4c3)c3cc4ccccc4cc3-c3ccccc3)cc2)cc1 ; c1ccc(-c2ccc(-n3c4ccccc4c4ccc(N(c5ccccc5)c5cc(-c6cccc7ccccc67)ccc5-c5ccccc5)cc43)cc2-c2ccccc2)cc1]

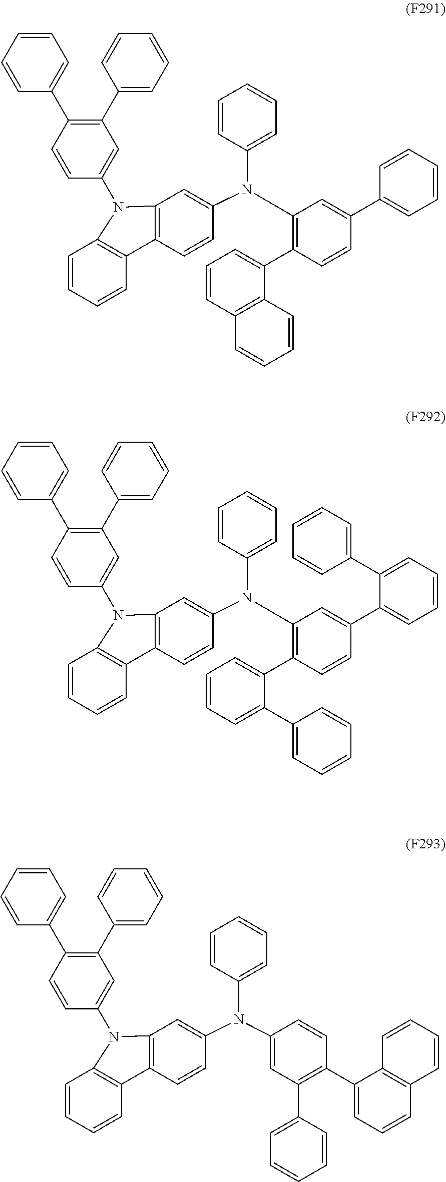 [SMILES: c1ccc(-c2ccc(-c3cccc4ccccc34)c(N(c3ccccc3)c3ccc4c5ccccc5n(-c5ccc(-c6ccccc6)c(-c6ccccc6)c5)c4c3)c2)cc1 ; c1ccc(-c2ccccc2-c2ccc(-c3ccccc3-c3ccccc3)c(N(c3ccccc3)c3ccc4c5ccccc5n(-c5ccc(-c6ccccc6)c(-c6ccccc6)c5)c4c3)c2)cc1 ; c1ccc(-c2ccc(-n3c4ccccc4c4ccc(N(c5ccccc5)c5ccc(-c6cccc7ccccc67)c(-c6ccccc6)c5)cc43)cc2-c2ccccc2)cc1]

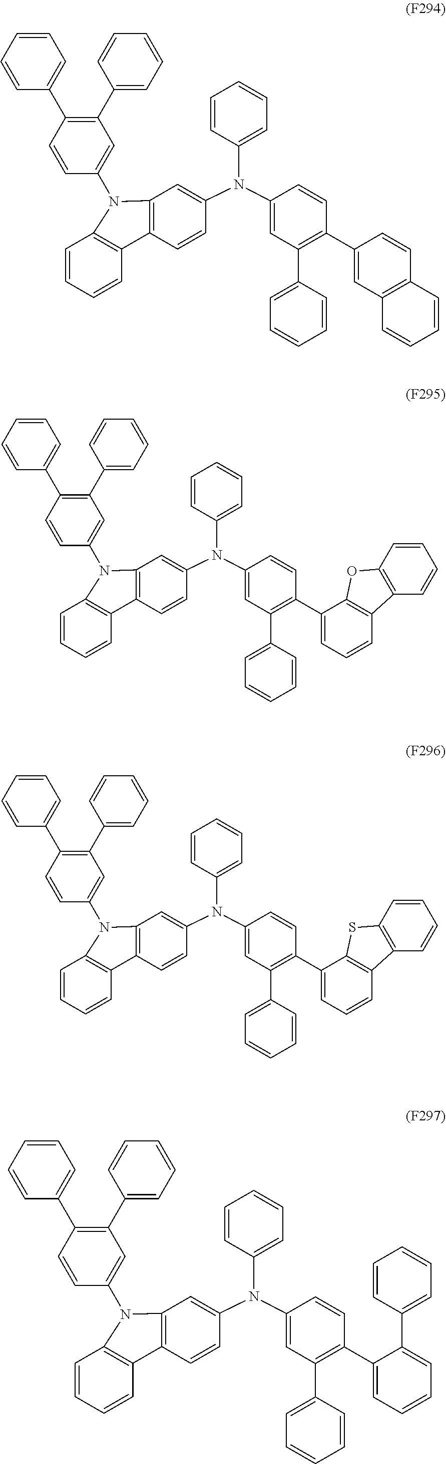 [SMILES: c1ccc(-c2ccc(-n3c4ccccc4c4ccc(N(c5ccccc5)c5ccc(-c6ccc7ccccc7c6)c(-c6ccccc6)c5)cc43)cc2-c2ccccc2)cc1 ; c1ccc(-c2ccc(-n3c4ccccc4c4ccc(N(c5ccccc5)c5ccc(-c6cccc7c6oc6ccccc67)c(-c6ccccc6)c5)cc43)cc2-c2ccccc2)cc1 ; c1ccc(-c2ccc(-n3c4ccccc4c4ccc(N(c5ccccc5)c5ccc(-c6cccc7c6sc6ccccc67)c(-c6ccccc6)c5)cc43)cc2-c2ccccc2)cc1 ; c1ccc(-c2ccc(-n3c4ccccc4c4ccc(N(c5ccccc5)c5ccc(-c6ccccc6-c6ccccc6)c(-c6ccccc6)c5)cc43)cc2-c2ccccc2)cc1]

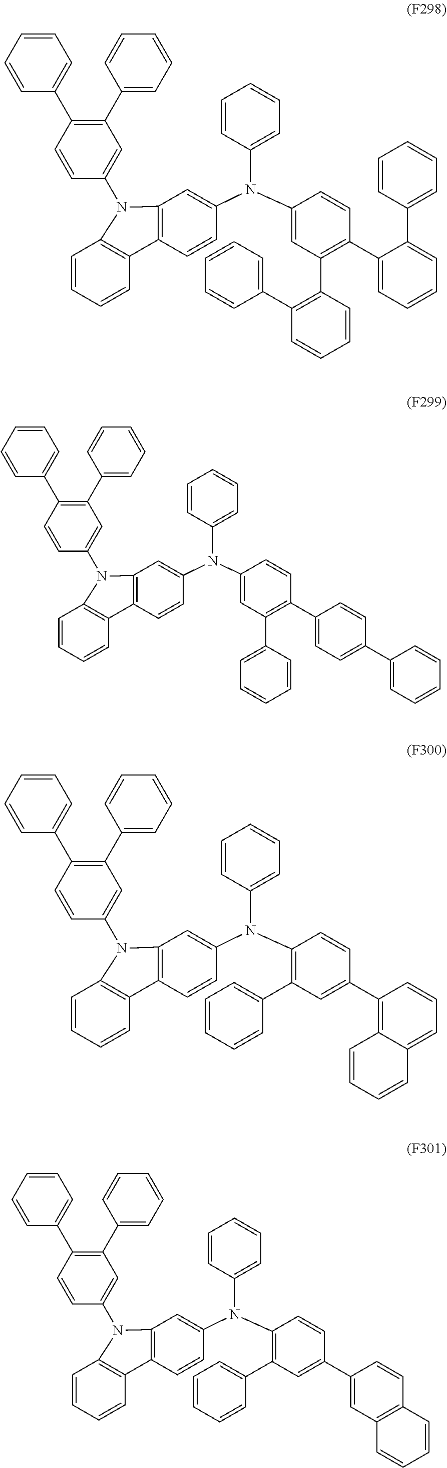 [SMILES: c1ccc(-c2ccc(-n3c4ccccc4c4ccc(N(c5ccccc5)c5ccc(-c6ccccc6-c6ccccc6)c(-c6ccccc6-c6ccccc6)c5)cc43)cc2-c2ccccc2)cc1 ; c1ccc(-c2ccc(-c3ccc(N(c4ccccc4)c4ccc5c6ccccc6n(-c6ccc(-c7ccccc7)c(-c7ccccc7)c6)c5c4)cc3-c3ccccc3)cc2)cc1 ; c1ccc(-c2ccc(-n3c4ccccc4c4ccc(N(c5ccccc5)c5ccc(-c6cccc7ccccc67)cc5-c5ccccc5)cc43)cc2-c2ccccc2)cc1 ; c1ccc(-c2ccc(-n3c4ccccc4c4ccc(N(c5ccccc5)c5ccc(-c6ccc7ccccc7c6)cc5-c5ccccc5)cc43)cc2-c2ccccc2)cc1]

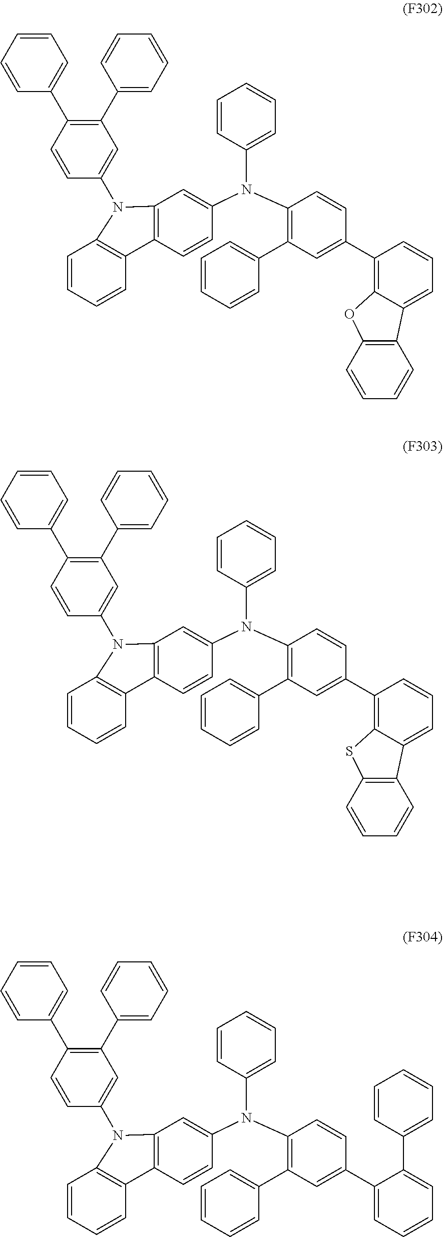 [SMILES: c1ccc(-c2ccc(-n3c4ccccc4c4ccc(N(c5ccccc5)c5ccc(-c6cccc7c6oc6ccccc67)cc5-c5ccccc5)cc43)cc2-c2ccccc2)cc1 ; c1ccc(-c2ccc(-n3c4ccccc4c4ccc(N(c5ccccc5)c5ccc(-c6cccc7c6sc6ccccc67)cc5-c5ccccc5)cc43)cc2-c2ccccc2)cc1 ; c1ccc(-c2ccccc2-c2ccc(N(c3ccccc3)c3ccc4c5ccccc5n(-c5ccc(-c6ccccc6)c(-c6ccccc6)c5)c4c3)c(-c3ccccc3)c2)cc1]

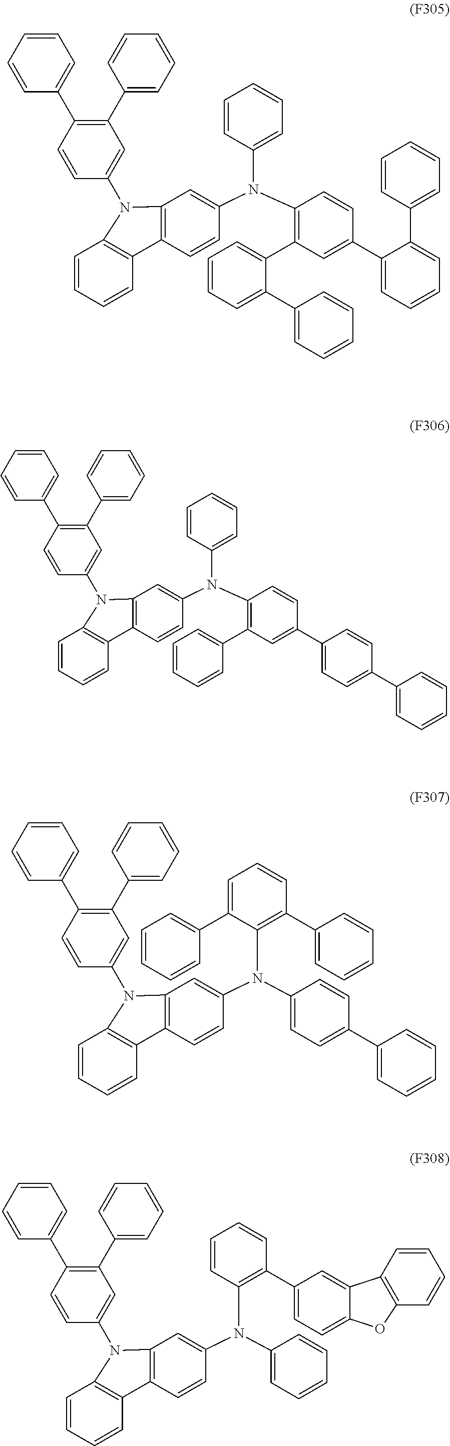 [SMILES: c1ccc(-c2ccccc2-c2ccc(N(c3ccccc3)c3ccc4c5ccccc5n(-c5ccc(-c6ccccc6)c(-c6ccccc6)c5)c4c3)c(-c3ccccc3-c3ccccc3)c2)cc1 ; c1ccc(-c2ccc(-c3ccc(N(c4ccccc4)c4ccc5c6ccccc6n(-c6ccc(-c7ccccc7)c(-c7ccccc7)c6)c5c4)c(-c4ccccc4)c3)cc2)cc1 ; c1ccc(-c2ccc(N(c3ccc4c5ccccc5n(-c5ccc(-c6ccccc6)c(-c6ccccc6)c5)c4c3)c3c(-c4ccccc4)cccc3-c3ccccc3)cc2)cc1 ; c1ccc(-c2ccc(-n3c4ccccc4c4ccc(N(c5ccccc5)c5ccccc5-c5ccc6oc7ccccc7c6c5)cc43)cc2-c2ccccc2)cc1]

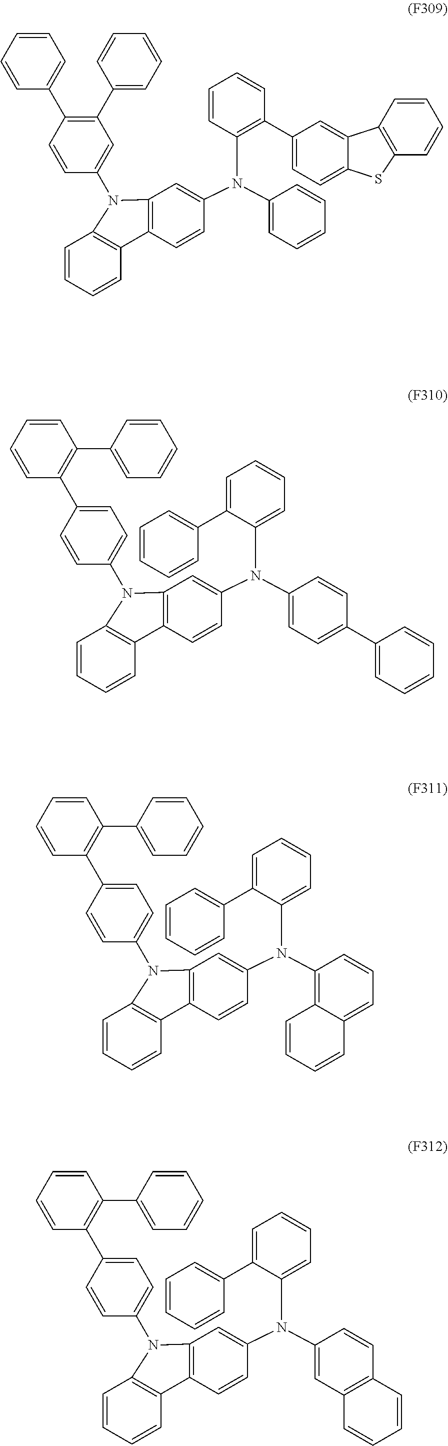 [SMILES: c1ccc(-c2ccc(-n3c4ccccc4c4ccc(N(c5ccccc5)c5ccccc5-c5ccc6sc7ccccc7c6c5)cc43)cc2-c2ccccc2)cc1 ; c1ccc(-c2ccc(N(c3ccc4c5ccccc5n(-c5ccc(-c6ccccc6-c6ccccc6)cc5)c4c3)c3ccccc3-c3ccccc3)cc2)cc1 ; c1ccc(-c2ccccc2-c2ccc(-n3c4ccccc4c4ccc(N(c5ccccc5-c5ccccc5)c5cccc6ccccc56)cc43)cc2)cc1 ; c1ccc(-c2ccccc2-c2ccc(-n3c4ccccc4c4ccc(N(c5ccc6ccccc6c5)c5ccccc5-c5ccccc5)cc43)cc2)cc1]

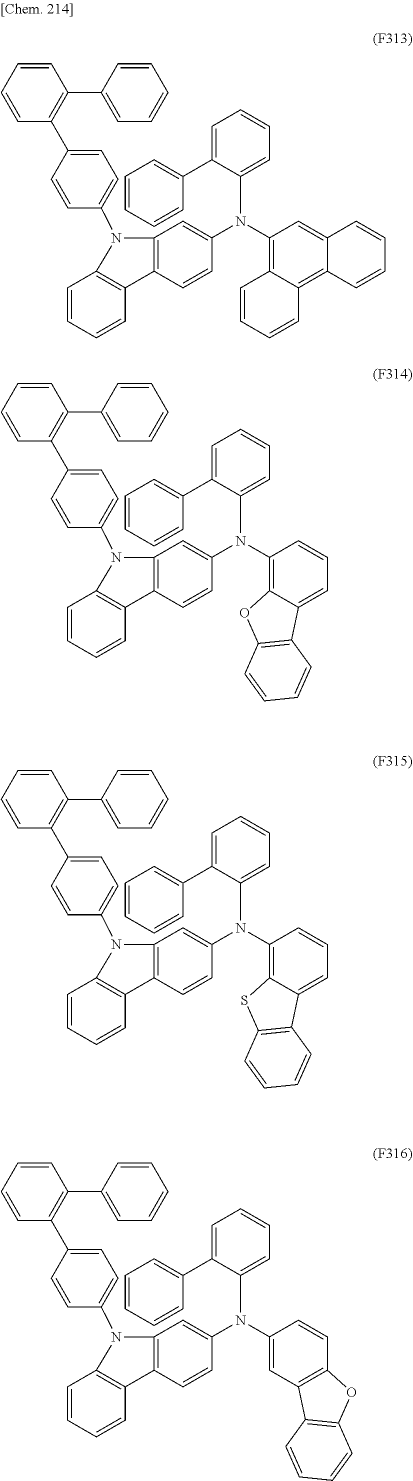 [SMILES: c1ccc(-c2ccccc2-c2ccc(-n3c4ccccc4c4ccc(N(c5ccccc5-c5ccccc5)c5cc6ccccc6c6ccccc56)cc43)cc2)cc1 ; c1ccc(-c2ccccc2-c2ccc(-n3c4ccccc4c4ccc(N(c5ccccc5-c5ccccc5)c5cccc6c5oc5ccccc56)cc43)cc2)cc1 ; c1ccc(-c2ccccc2-c2ccc(-n3c4ccccc4c4ccc(N(c5ccccc5-c5ccccc5)c5cccc6c5sc5ccccc56)cc43)cc2)cc1 ; c1ccc(-c2ccccc2-c2ccc(-n3c4ccccc4c4ccc(N(c5ccc6oc7ccccc7c6c5)c5ccccc5-c5ccccc5)cc43)cc2)cc1]

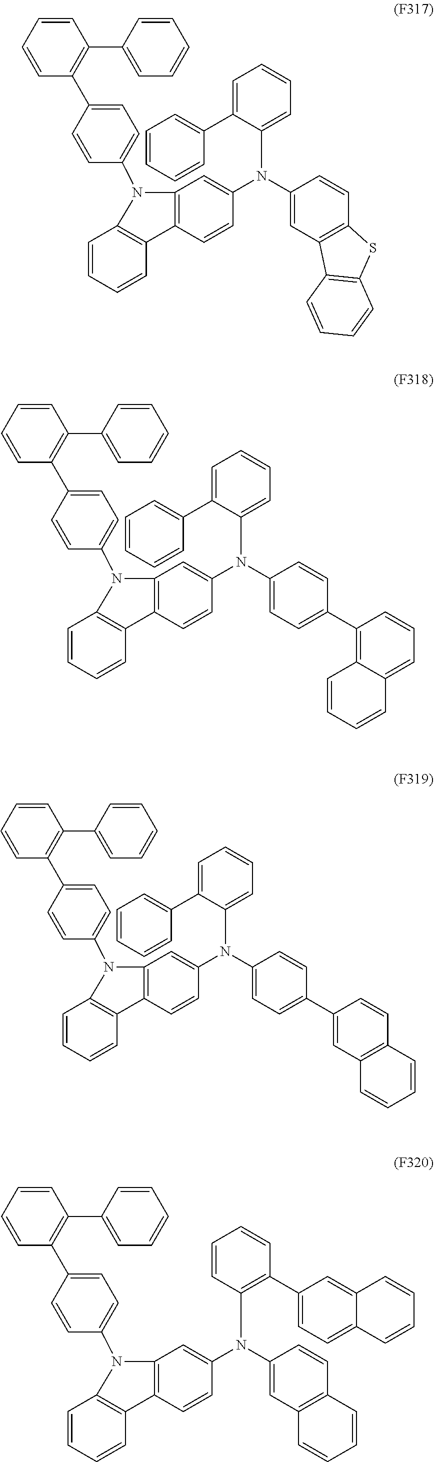 [SMILES: c1ccc(-c2ccccc2-c2ccc(-n3c4ccccc4c4ccc(N(c5ccc6sc7ccccc7c6c5)c5ccccc5-c5ccccc5)cc43)cc2)cc1 ; c1ccc(-c2ccccc2-c2ccc(-n3c4ccccc4c4ccc(N(c5ccc(-c6cccc7ccccc67)cc5)c5ccccc5-c5ccccc5)cc43)cc2)cc1 ; c1ccc(-c2ccccc2-c2ccc(-n3c4ccccc4c4ccc(N(c5ccc(-c6ccc7ccccc7c6)cc5)c5ccccc5-c5ccccc5)cc43)cc2)cc1 ; c1ccc(-c2ccccc2-c2ccc(-n3c4ccccc4c4ccc(N(c5ccc6ccccc6c5)c5ccccc5-c5ccc6ccccc6c5)cc43)cc2)cc1]

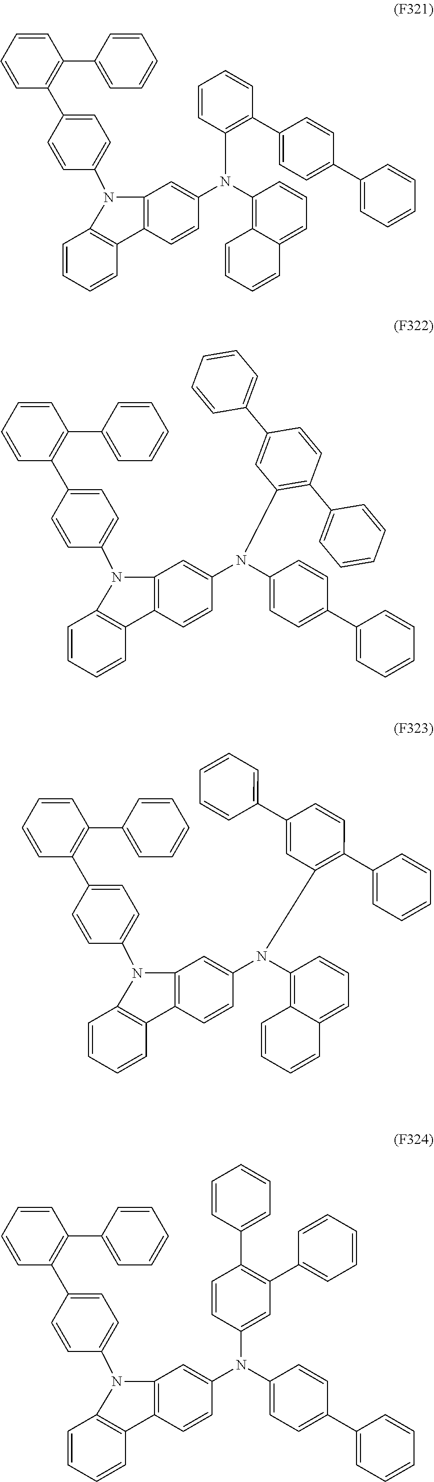 [SMILES: c1ccc(-c2ccc(-c3ccccc3N(c3ccc4c5ccccc5n(-c5ccc(-c6ccccc6-c6ccccc6)cc5)c4c3)c3cccc4ccccc34)cc2)cc1 ; c1ccc(-c2ccc(N(c3ccc4c5ccccc5n(-c5ccc(-c6ccccc6-c6ccccc6)cc5)c4c3)c3cc(-c4ccccc4)ccc3-c3ccccc3)cc2)cc1 ; c1ccc(-c2ccc(-c3ccccc3)c(N(c3ccc4c5ccccc5n(-c5ccc(-c6ccccc6-c6ccccc6)cc5)c4c3)c3cccc4ccccc34)c2)cc1 ; c1ccc(-c2ccc(N(c3ccc(-c4ccccc4)c(-c4ccccc4)c3)c3ccc4c5ccccc5n(-c5ccc(-c6ccccc6-c6ccccc6)cc5)c4c3)cc2)cc1]

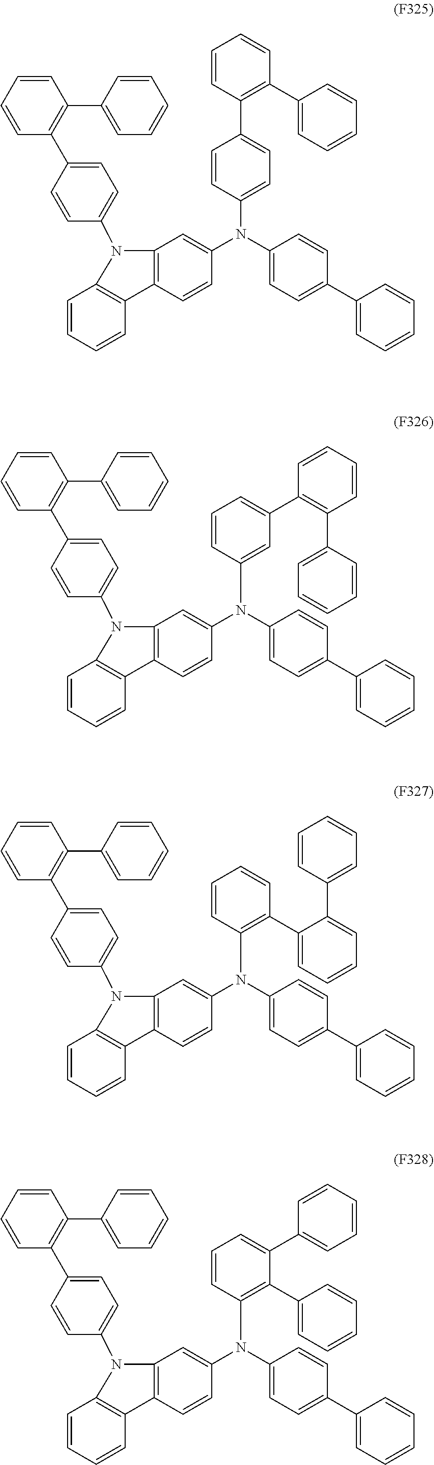 [SMILES: c1ccc(-c2ccc(N(c3ccc(-c4ccccc4-c4ccccc4)cc3)c3ccc4c5ccccc5n(-c5ccc(-c6ccccc6-c6ccccc6)cc5)c4c3)cc2)cc1 ; c1ccc(-c2ccc(N(c3cccc(-c4ccccc4-c4ccccc4)c3)c3ccc4c5ccccc5n(-c5ccc(-c6ccccc6-c6ccccc6)cc5)c4c3)cc2)cc1 ; c1ccc(-c2ccc(N(c3ccc4c5ccccc5n(-c5ccc(-c6ccccc6-c6ccccc6)cc5)c4c3)c3ccccc3-c3ccccc3-c3ccccc3)cc2)cc1 ; c1ccc(-c2ccc(N(c3ccc4c5ccccc5n(-c5ccc(-c6ccccc6-c6ccccc6)cc5)c4c3)c3cccc(-c4ccccc4)c3-c3ccccc3)cc2)cc1]

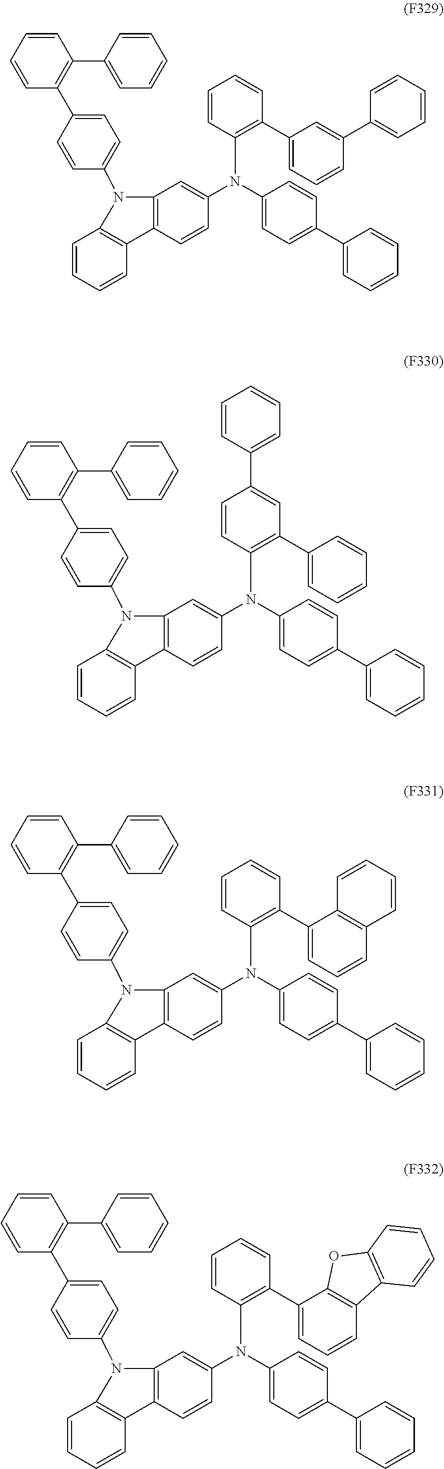 [SMILES: c1ccc(-c2ccc(N(c3ccc4c5ccccc5n(-c5ccc(-c6ccccc6-c6ccccc6)cc5)c4c3)c3ccccc3-c3cccc(-c4ccccc4)c3)cc2)cc1 ; c1ccc(-c2ccc(N(c3ccc4c5ccccc5n(-c5ccc(-c6ccccc6-c6ccccc6)cc5)c4c3)c3ccc(-c4ccccc4)cc3-c3ccccc3)cc2)cc1 ; c1ccc(-c2ccc(N(c3ccc4c5ccccc5n(-c5ccc(-c6ccccc6-c6ccccc6)cc5)c4c3)c3ccccc3-c3cccc4ccccc34)cc2)cc1 ; c1ccc(-c2ccc(N(c3ccc4c5ccccc5n(-c5ccc(-c6ccccc6-c6ccccc6)cc5)c4c3)c3ccccc3-c3cccc4c3oc3ccccc34)cc2)cc1]

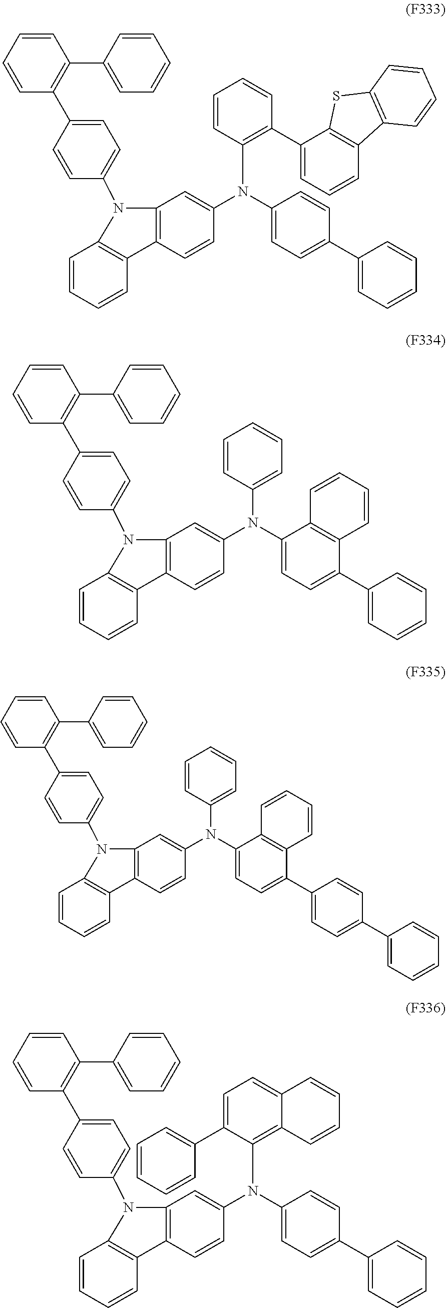 [SMILES: c1ccc(-c2ccc(N(c3ccc4c5ccccc5n(-c5ccc(-c6ccccc6-c6ccccc6)cc5)c4c3)c3ccccc3-c3cccc4c3sc3ccccc34)cc2)cc1 ; c1ccc(-c2ccccc2-c2ccc(-n3c4ccccc4c4ccc(N(c5ccccc5)c5ccc(-c6ccccc6)c6ccccc56)cc43)cc2)cc1 ; c1ccc(-c2ccc(-c3ccc(N(c4ccccc4)c4ccc5c6ccccc6n(-c6ccc(-c7ccccc7-c7ccccc7)cc6)c5c4)c4ccccc34)cc2)cc1 ; c1ccc(-c2ccc(N(c3ccc4c5ccccc5n(-c5ccc(-c6ccccc6-c6ccccc6)cc5)c4c3)c3c(-c4ccccc4)ccc4ccccc34)cc2)cc1]

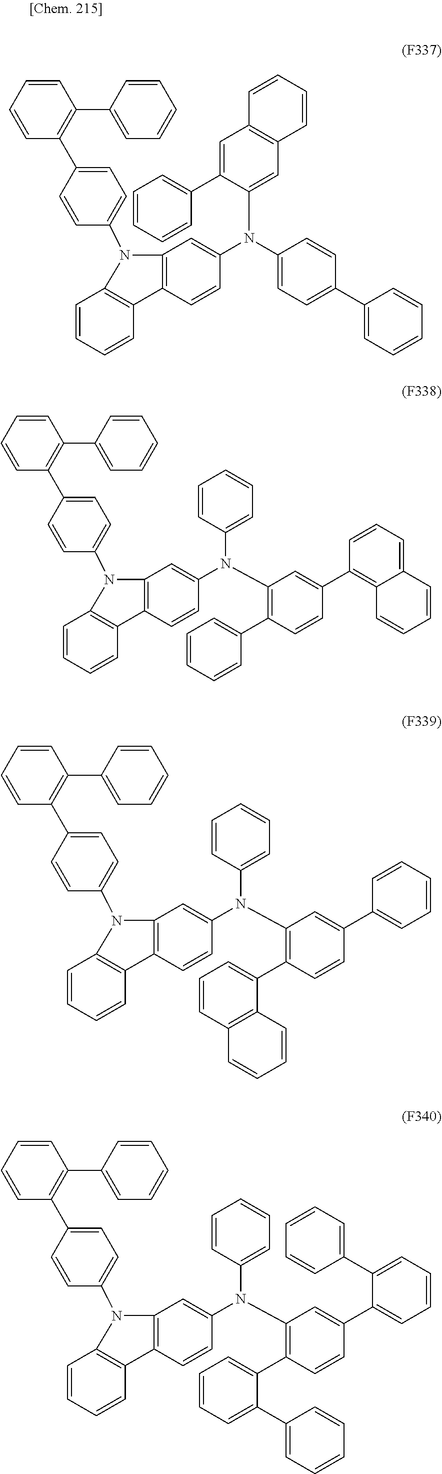 [SMILES: c1ccc(-c2ccc(N(c3ccc4c5ccccc5n(-c5ccc(-c6ccccc6-c6ccccc6)cc5)c4c3)c3cc4ccccc4cc3-c3ccccc3)cc2)cc1 ; c1ccc(-c2ccccc2-c2ccc(-n3c4ccccc4c4ccc(N(c5ccccc5)c5cc(-c6cccc7ccccc67)ccc5-c5ccccc5)cc43)cc2)cc1 ; c1ccc(-c2ccc(-c3cccc4ccccc34)c(N(c3ccccc3)c3ccc4c5ccccc5n(-c5ccc(-c6ccccc6-c6ccccc6)cc5)c4c3)c2)cc1 ; c1ccc(-c2ccccc2-c2ccc(-n3c4ccccc4c4ccc(N(c5ccccc5)c5cc(-c6ccccc6-c6ccccc6)ccc5-c5ccccc5-c5ccccc5)cc43)cc2)cc1]

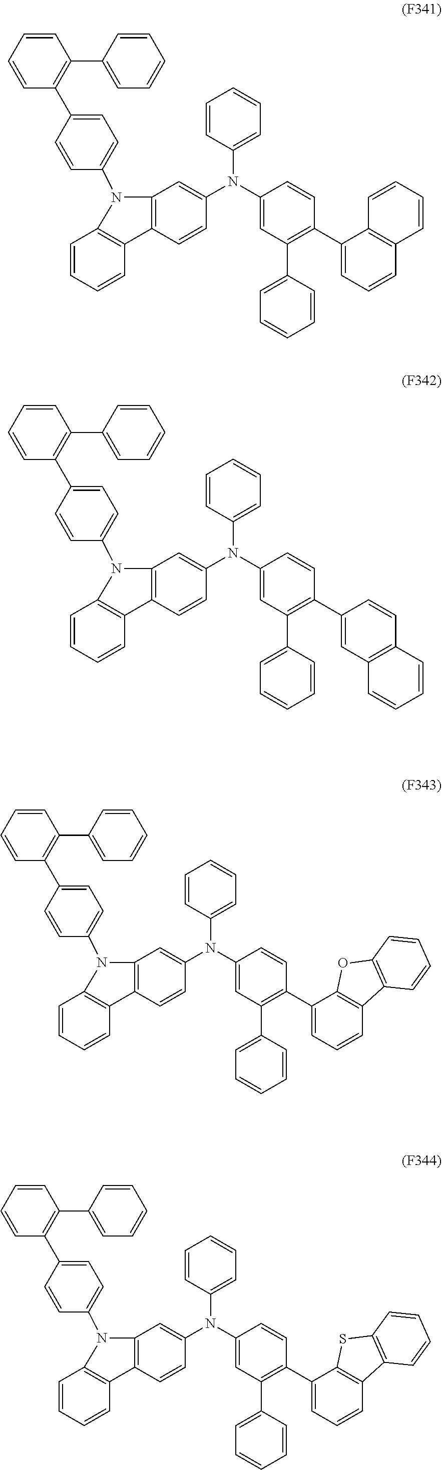 [SMILES: c1ccc(-c2ccccc2-c2ccc(-n3c4ccccc4c4ccc(N(c5ccccc5)c5ccc(-c6cccc7ccccc67)c(-c6ccccc6)c5)cc43)cc2)cc1 ; c1ccc(-c2ccccc2-c2ccc(-n3c4ccccc4c4ccc(N(c5ccccc5)c5ccc(-c6ccc7ccccc7c6)c(-c6ccccc6)c5)cc43)cc2)cc1 ; c1ccc(-c2ccccc2-c2ccc(-n3c4ccccc4c4ccc(N(c5ccccc5)c5ccc(-c6cccc7c6oc6ccccc67)c(-c6ccccc6)c5)cc43)cc2)cc1 ; c1ccc(-c2ccccc2-c2ccc(-n3c4ccccc4c4ccc(N(c5ccccc5)c5ccc(-c6cccc7c6sc6ccccc67)c(-c6ccccc6)c5)cc43)cc2)cc1]

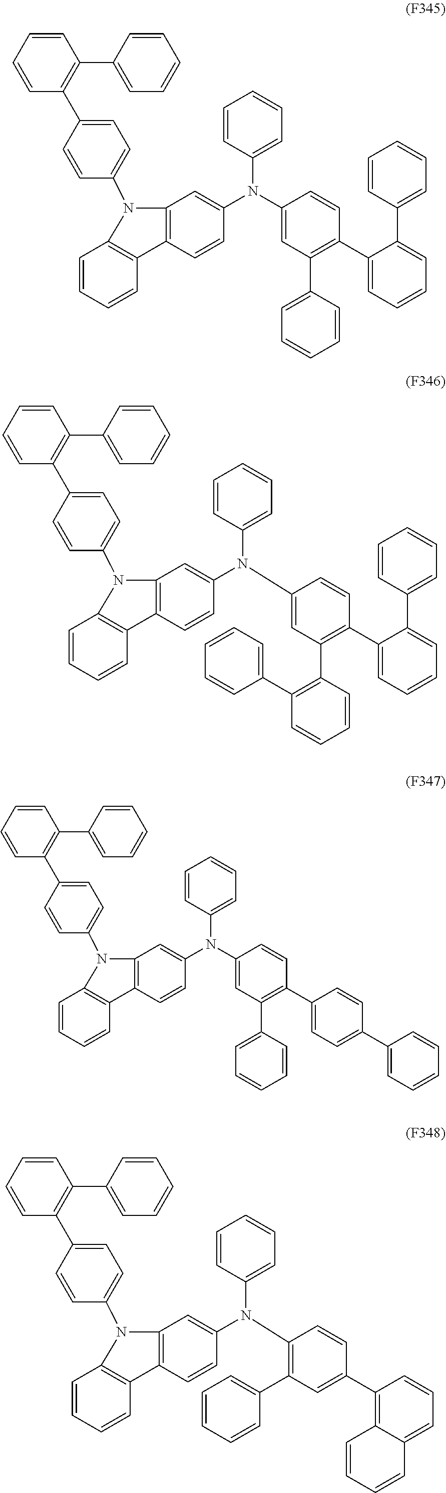 [SMILES: c1ccc(-c2ccccc2-c2ccc(-n3c4ccccc4c4ccc(N(c5ccccc5)c5ccc(-c6ccccc6-c6ccccc6)c(-c6ccccc6)c5)cc43)cc2)cc1 ; c1ccc(-c2ccccc2-c2ccc(-n3c4ccccc4c4ccc(N(c5ccccc5)c5ccc(-c6ccccc6-c6ccccc6)c(-c6ccccc6-c6ccccc6)c5)cc43)cc2)cc1 ; c1ccc(-c2ccc(-c3ccc(N(c4ccccc4)c4ccc5c6ccccc6n(-c6ccc(-c7ccccc7-c7ccccc7)cc6)c5c4)cc3-c3ccccc3)cc2)cc1 ; c1ccc(-c2ccccc2-c2ccc(-n3c4ccccc4c4ccc(N(c5ccccc5)c5ccc(-c6cccc7ccccc67)cc5-c5ccccc5)cc43)cc2)cc1]

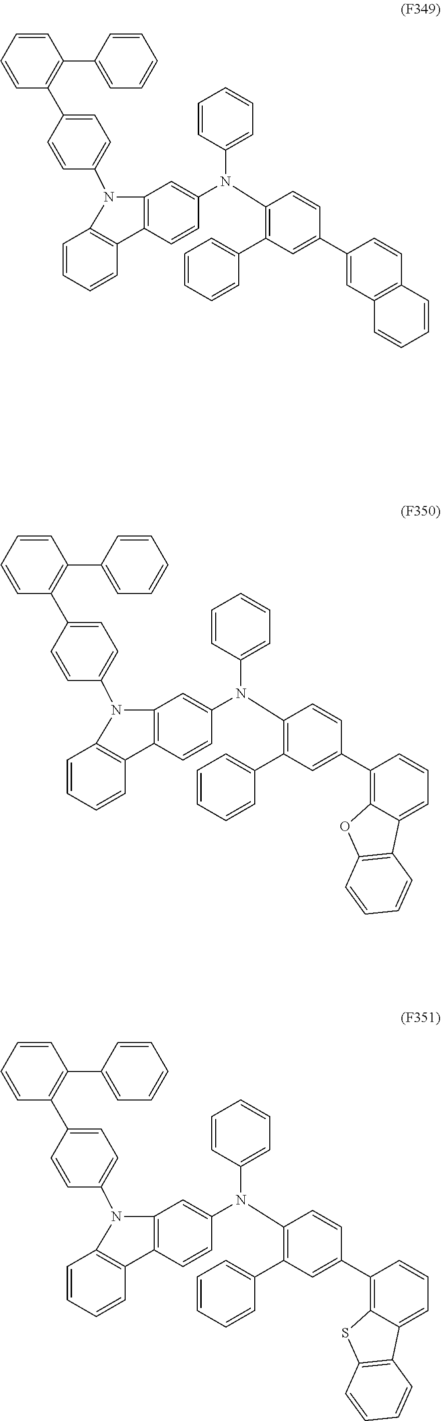 [SMILES: c1ccc(-c2ccccc2-c2ccc(-n3c4ccccc4c4ccc(N(c5ccccc5)c5ccc(-c6ccc7ccccc7c6)cc5-c5ccccc5)cc43)cc2)cc1 ; c1ccc(-c2ccccc2-c2ccc(-n3c4ccccc4c4ccc(N(c5ccccc5)c5ccc(-c6cccc7c6oc6ccccc67)cc5-c5ccccc5)cc43)cc2)cc1 ; c1ccc(-c2ccccc2-c2ccc(-n3c4ccccc4c4ccc(N(c5ccccc5)c5ccc(-c6cccc7c6sc6ccccc67)cc5-c5ccccc5)cc43)cc2)cc1]

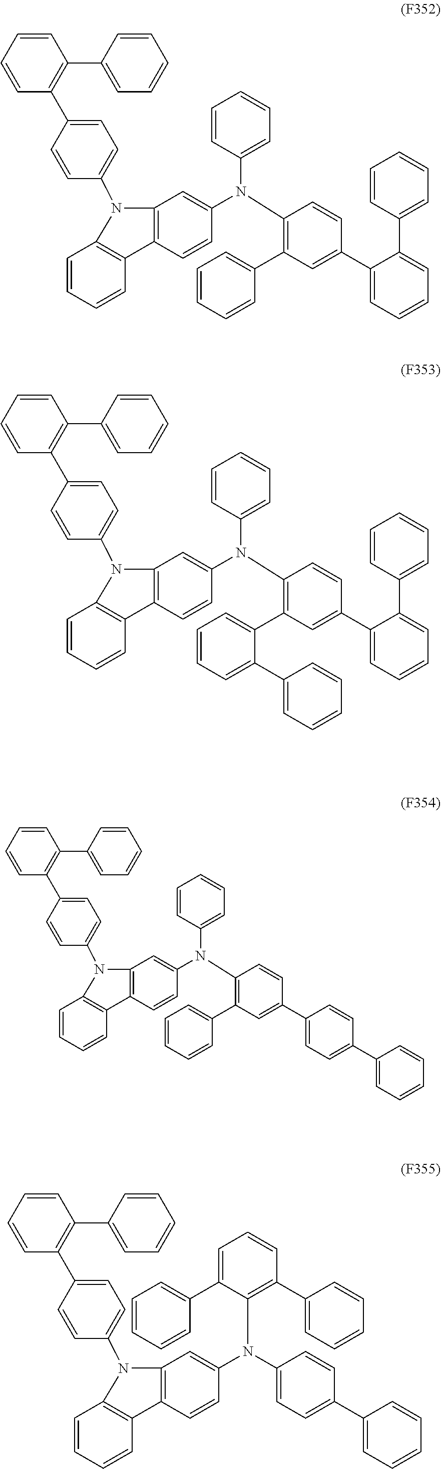 [SMILES: c1ccc(-c2ccccc2-c2ccc(-n3c4ccccc4c4ccc(N(c5ccccc5)c5ccc(-c6ccccc6-c6ccccc6)cc5-c5ccccc5)cc43)cc2)cc1 ; c1ccc(-c2ccccc2-c2ccc(-n3c4ccccc4c4ccc(N(c5ccccc5)c5ccc(-c6ccccc6-c6ccccc6)cc5-c5ccccc5-c5ccccc5)cc43)cc2)cc1 ; c1ccc(-c2ccc(-c3ccc(N(c4ccccc4)c4ccc5c6ccccc6n(-c6ccc(-c7ccccc7-c7ccccc7)cc6)c5c4)c(-c4ccccc4)c3)cc2)cc1 ; c1ccc(-c2ccc(N(c3ccc4c5ccccc5n(-c5ccc(-c6ccccc6-c6ccccc6)cc5)c4c3)c3c(-c4ccccc4)cccc3-c3ccccc3)cc2)cc1]

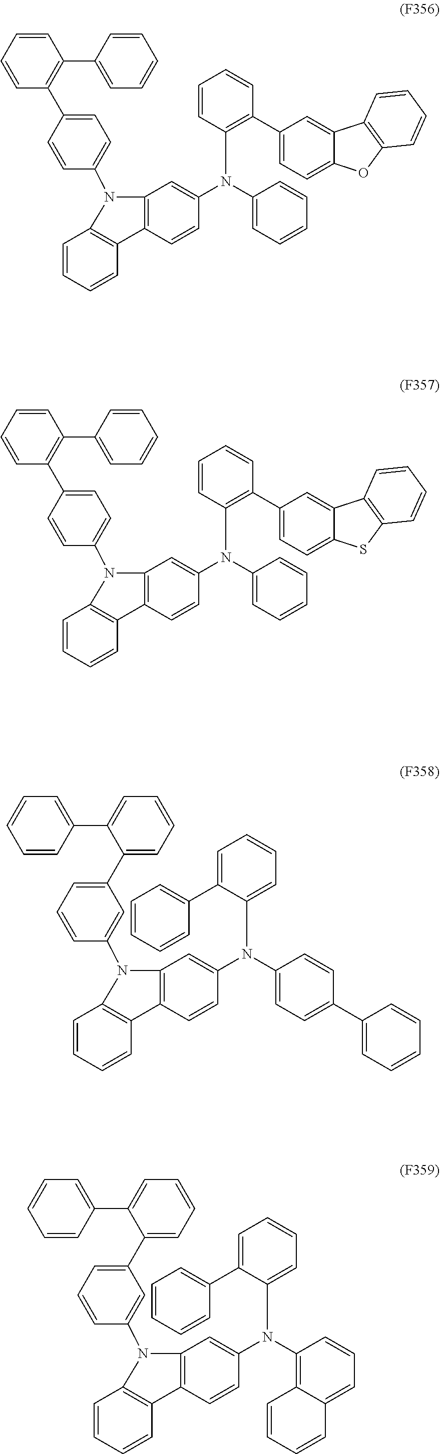 [SMILES: c1ccc(-c2ccccc2-c2ccc(-n3c4ccccc4c4ccc(N(c5ccccc5)c5ccccc5-c5ccc6oc7ccccc7c6c5)cc43)cc2)cc1 ; c1ccc(-c2ccccc2-c2ccc(-n3c4ccccc4c4ccc(N(c5ccccc5)c5ccccc5-c5ccc6sc7ccccc7c6c5)cc43)cc2)cc1 ; c1ccc(-c2ccc(N(c3ccc4c5ccccc5n(-c5cccc(-c6ccccc6-c6ccccc6)c5)c4c3)c3ccccc3-c3ccccc3)cc2)cc1 ; c1ccc(-c2ccccc2-c2cccc(-n3c4ccccc4c4ccc(N(c5ccccc5-c5ccccc5)c5cccc6ccccc56)cc43)c2)cc1]

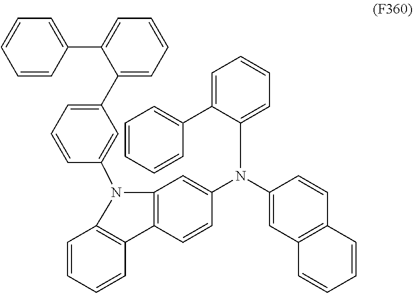 [SMILES: c1ccc(-c2ccccc2-c2cccc(-n3c4ccccc4c4ccc(N(c5ccc6ccccc6c5)c5ccccc5-c5ccccc5)cc43)c2)cc1]

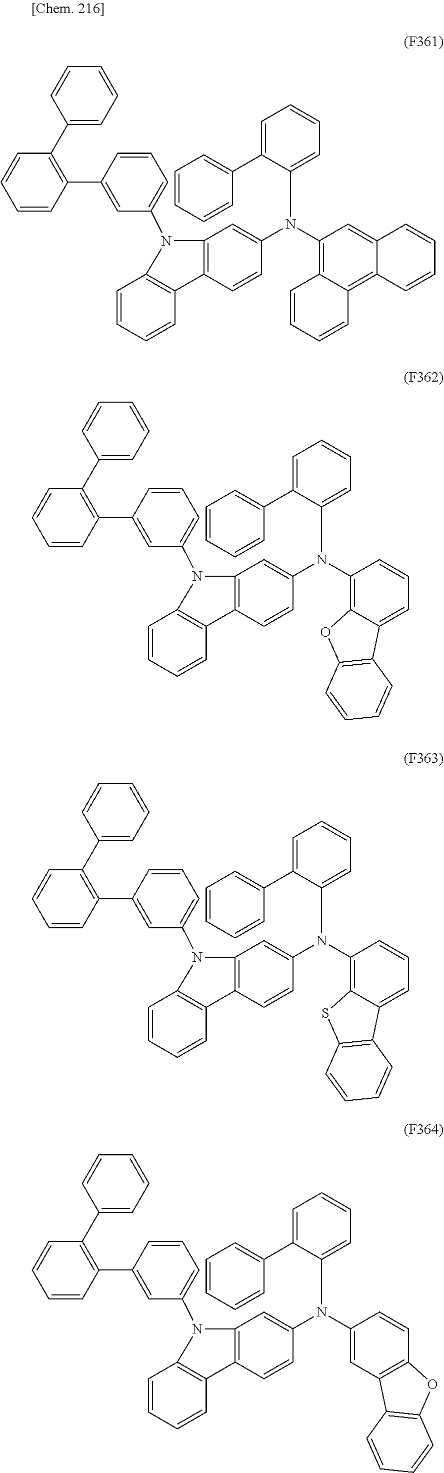 [SMILES: c1ccc(-c2ccccc2-c2cccc(-n3c4ccccc4c4ccc(N(c5ccccc5-c5ccccc5)c5cc6ccccc6c6ccccc56)cc43)c2)cc1 ; c1ccc(-c2ccccc2-c2cccc(-n3c4ccccc4c4ccc(N(c5ccccc5-c5ccccc5)c5cccc6c5oc5ccccc56)cc43)c2)cc1 ; c1ccc(-c2ccccc2-c2cccc(-n3c4ccccc4c4ccc(N(c5ccccc5-c5ccccc5)c5cccc6c5sc5ccccc56)cc43)c2)cc1 ; c1ccc(-c2ccccc2-c2cccc(-n3c4ccccc4c4ccc(N(c5ccc6oc7ccccc7c6c5)c5ccccc5-c5ccccc5)cc43)c2)cc1]

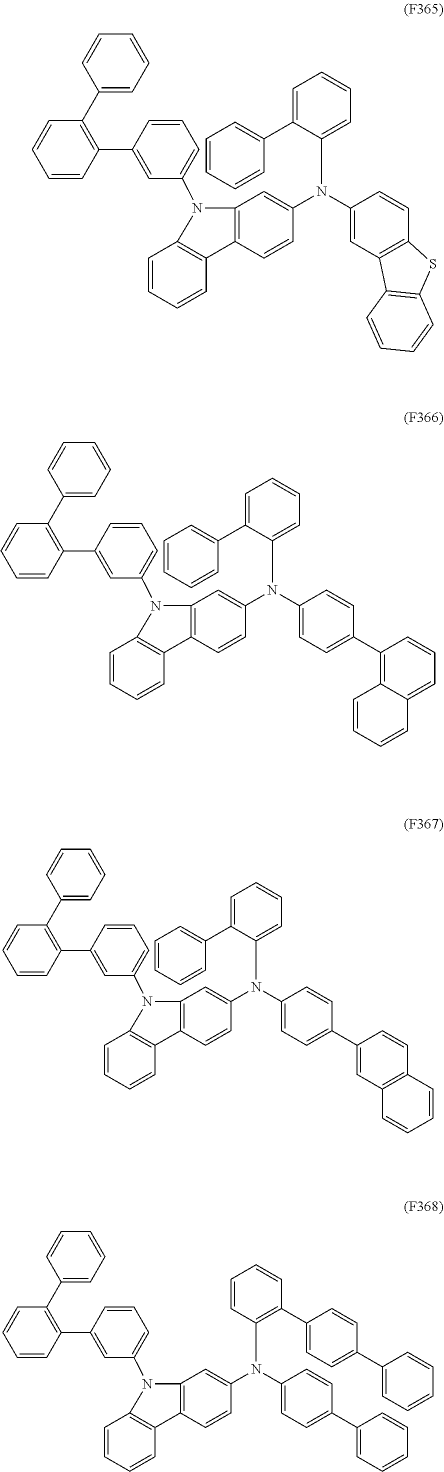 [SMILES: c1ccc(-c2ccccc2-c2cccc(-n3c4ccccc4c4ccc(N(c5ccc6sc7ccccc7c6c5)c5ccccc5-c5ccccc5)cc43)c2)cc1 ; c1ccc(-c2ccccc2-c2cccc(-n3c4ccccc4c4ccc(N(c5ccc(-c6cccc7ccccc67)cc5)c5ccccc5-c5ccccc5)cc43)c2)cc1 ; c1ccc(-c2ccccc2-c2cccc(-n3c4ccccc4c4ccc(N(c5ccc(-c6ccc7ccccc7c6)cc5)c5ccccc5-c5ccccc5)cc43)c2)cc1 ; c1ccc(-c2ccc(-c3ccccc3N(c3ccc(-c4ccccc4)cc3)c3ccc4c5ccccc5n(-c5cccc(-c6ccccc6-c6ccccc6)c5)c4c3)cc2)cc1]

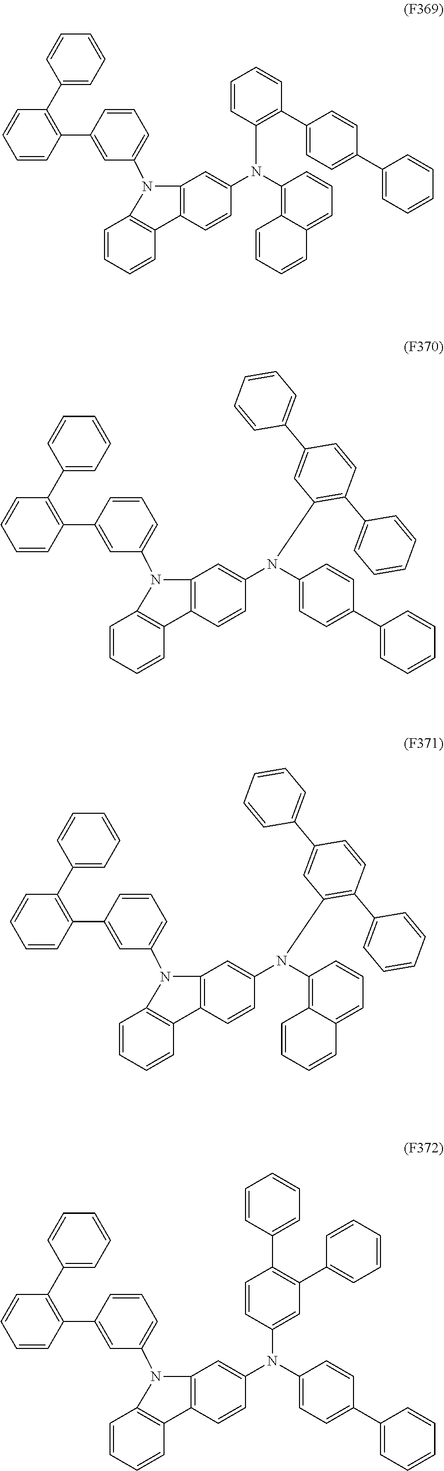 [SMILES: c1ccc(-c2ccc(-c3ccccc3N(c3ccc4c5ccccc5n(-c5cccc(-c6ccccc6-c6ccccc6)c5)c4c3)c3cccc4ccccc34)cc2)cc1 ; c1ccc(-c2ccc(N(c3ccc4c5ccccc5n(-c5cccc(-c6ccccc6-c6ccccc6)c5)c4c3)c3cc(-c4ccccc4)ccc3-c3ccccc3)cc2)cc1 ; c1ccc(-c2ccc(-c3ccccc3)c(N(c3ccc4c5ccccc5n(-c5cccc(-c6ccccc6-c6ccccc6)c5)c4c3)c3cccc4ccccc34)c2)cc1 ; c1ccc(-c2ccc(N(c3ccc(-c4ccccc4)c(-c4ccccc4)c3)c3ccc4c5ccccc5n(-c5cccc(-c6ccccc6-c6ccccc6)c5)c4c3)cc2)cc1]

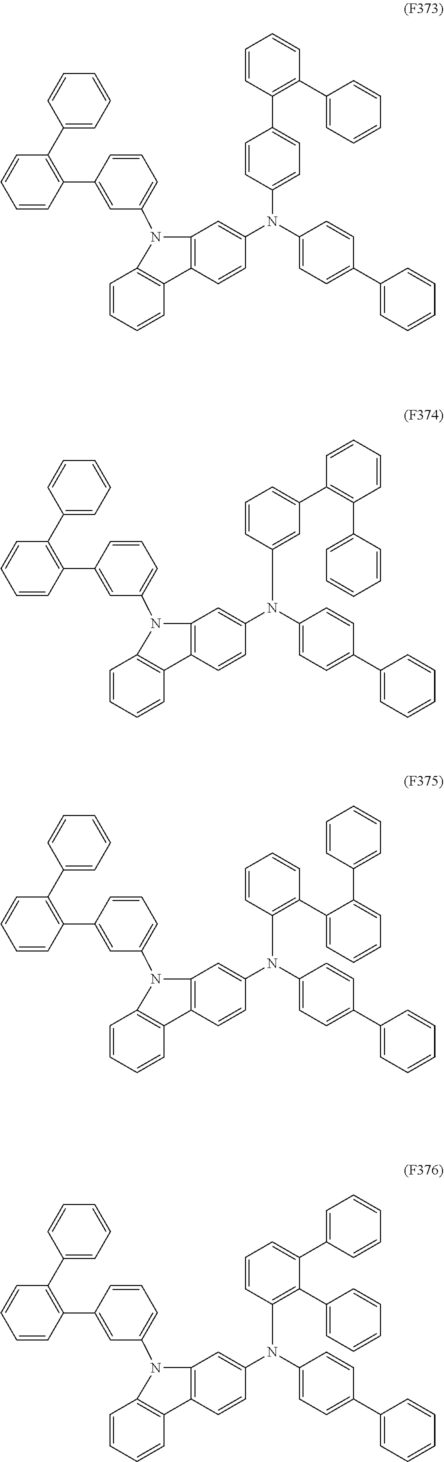 [SMILES: c1ccc(-c2ccc(N(c3ccc(-c4ccccc4-c4ccccc4)cc3)c3ccc4c5ccccc5n(-c5cccc(-c6ccccc6-c6ccccc6)c5)c4c3)cc2)cc1 ; c1ccc(-c2ccc(N(c3cccc(-c4ccccc4-c4ccccc4)c3)c3ccc4c5ccccc5n(-c5cccc(-c6ccccc6-c6ccccc6)c5)c4c3)cc2)cc1 ; c1ccc(-c2ccc(N(c3ccc4c5ccccc5n(-c5cccc(-c6ccccc6-c6ccccc6)c5)c4c3)c3ccccc3-c3ccccc3-c3ccccc3)cc2)cc1 ; c1ccc(-c2ccc(N(c3ccc4c5ccccc5n(-c5cccc(-c6ccccc6-c6ccccc6)c5)c4c3)c3cccc(-c4ccccc4)c3-c3ccccc3)cc2)cc1]

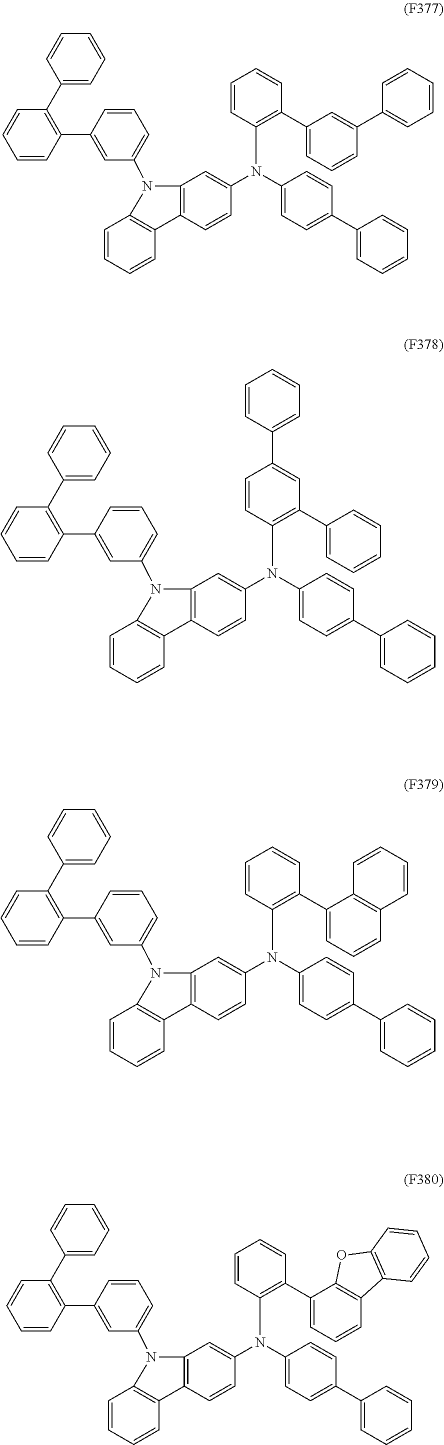 [SMILES: c1ccc(-c2ccc(N(c3ccc4c5ccccc5n(-c5cccc(-c6ccccc6-c6ccccc6)c5)c4c3)c3ccccc3-c3cccc(-c4ccccc4)c3)cc2)cc1 ; c1ccc(-c2ccc(N(c3ccc4c5ccccc5n(-c5cccc(-c6ccccc6-c6ccccc6)c5)c4c3)c3ccc(-c4ccccc4)cc3-c3ccccc3)cc2)cc1 ; c1ccc(-c2ccc(N(c3ccc4c5ccccc5n(-c5cccc(-c6ccccc6-c6ccccc6)c5)c4c3)c3ccccc3-c3cccc4ccccc34)cc2)cc1 ; c1ccc(-c2ccc(N(c3ccc4c5ccccc5n(-c5cccc(-c6ccccc6-c6ccccc6)c5)c4c3)c3ccccc3-c3cccc4c3oc3ccccc34)cc2)cc1]

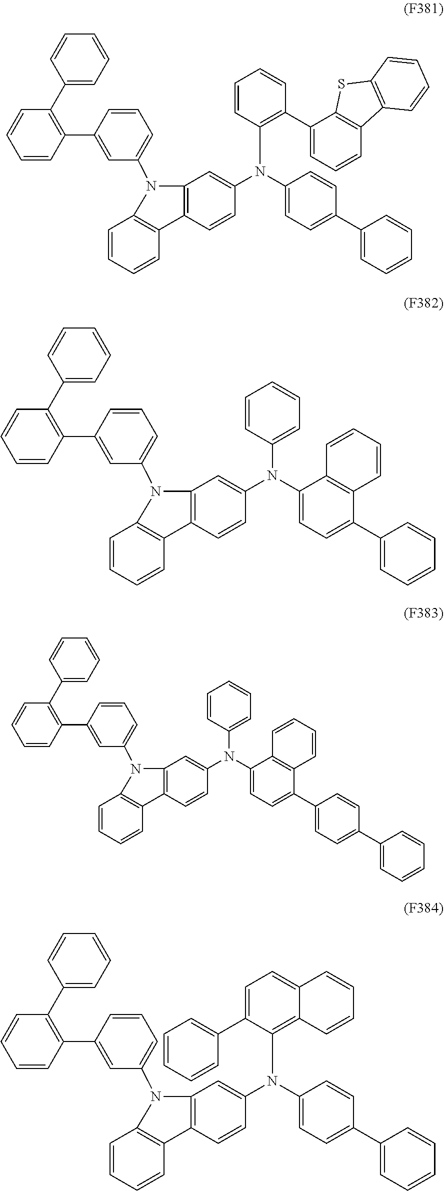 [SMILES: c1ccc(-c2ccc(N(c3ccc4c5ccccc5n(-c5cccc(-c6ccccc6-c6ccccc6)c5)c4c3)c3ccccc3-c3cccc4c3sc3ccccc34)cc2)cc1 ; c1ccc(-c2ccccc2-c2cccc(-n3c4ccccc4c4ccc(N(c5ccccc5)c5ccc(-c6ccccc6)c6ccccc56)cc43)c2)cc1 ; c1ccc(-c2ccc(-c3ccc(N(c4ccccc4)c4ccc5c6ccccc6n(-c6cccc(-c7ccccc7-c7ccccc7)c6)c5c4)c4ccccc34)cc2)cc1 ; c1ccc(-c2ccc(N(c3ccc4c5ccccc5n(-c5cccc(-c6ccccc6-c6ccccc6)c5)c4c3)c3c(-c4ccccc4)ccc4ccccc34)cc2)cc1]

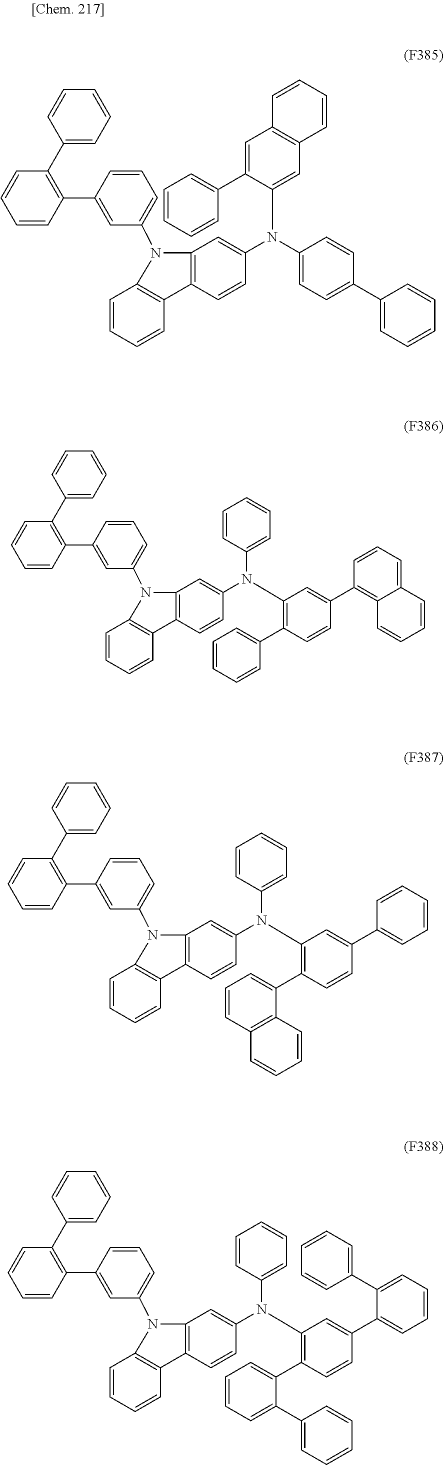 [SMILES: c1ccc(-c2ccc(N(c3ccc4c5ccccc5n(-c5cccc(-c6ccccc6-c6ccccc6)c5)c4c3)c3cc4ccccc4cc3-c3ccccc3)cc2)cc1 ; c1ccc(-c2ccccc2-c2cccc(-n3c4ccccc4c4ccc(N(c5ccccc5)c5cc(-c6cccc7ccccc67)ccc5-c5ccccc5)cc43)c2)cc1 ; c1ccc(-c2ccc(-c3cccc4ccccc34)c(N(c3ccccc3)c3ccc4c5ccccc5n(-c5cccc(-c6ccccc6-c6ccccc6)c5)c4c3)c2)cc1 ; c1ccc(-c2ccccc2-c2cccc(-n3c4ccccc4c4ccc(N(c5ccccc5)c5cc(-c6ccccc6-c6ccccc6)ccc5-c5ccccc5-c5ccccc5)cc43)c2)cc1]

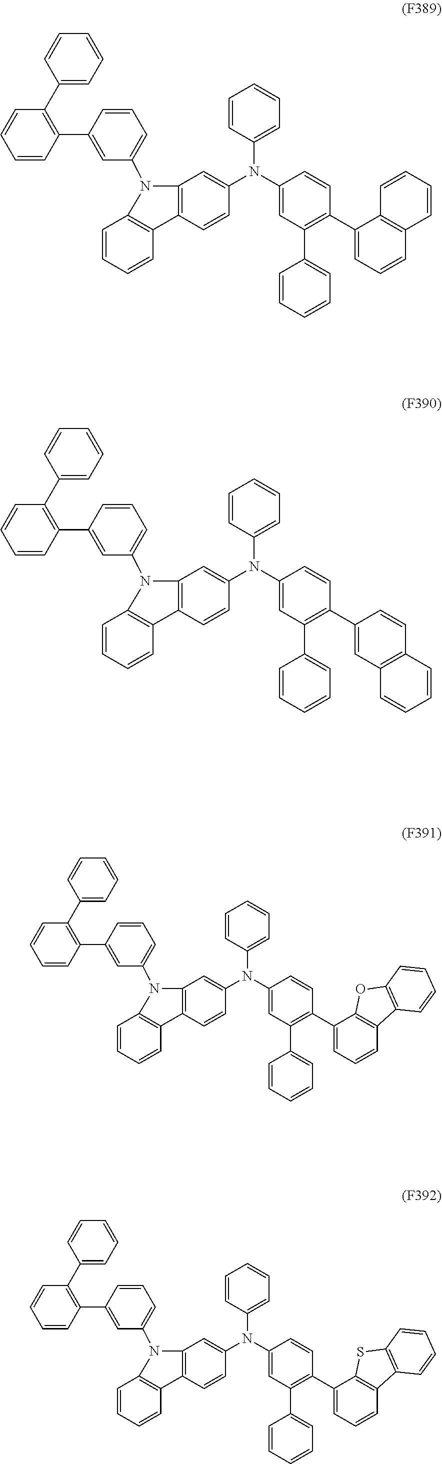 [SMILES: c1ccc(-c2ccccc2-c2cccc(-n3c4ccccc4c4ccc(N(c5ccccc5)c5ccc(-c6cccc7ccccc67)c(-c6ccccc6)c5)cc43)c2)cc1 ; c1ccc(-c2ccccc2-c2cccc(-n3c4ccccc4c4ccc(N(c5ccccc5)c5ccc(-c6ccc7ccccc7c6)c(-c6ccccc6)c5)cc43)c2)cc1 ; c1ccc(-c2ccccc2-c2cccc(-n3c4ccccc4c4ccc(N(c5ccccc5)c5ccc(-c6cccc7c6oc6ccccc67)c(-c6ccccc6)c5)cc43)c2)cc1 ; c1ccc(-c2ccccc2-c2cccc(-n3c4ccccc4c4ccc(N(c5ccccc5)c5ccc(-c6cccc7c6sc6ccccc67)c(-c6ccccc6)c5)cc43)c2)cc1]

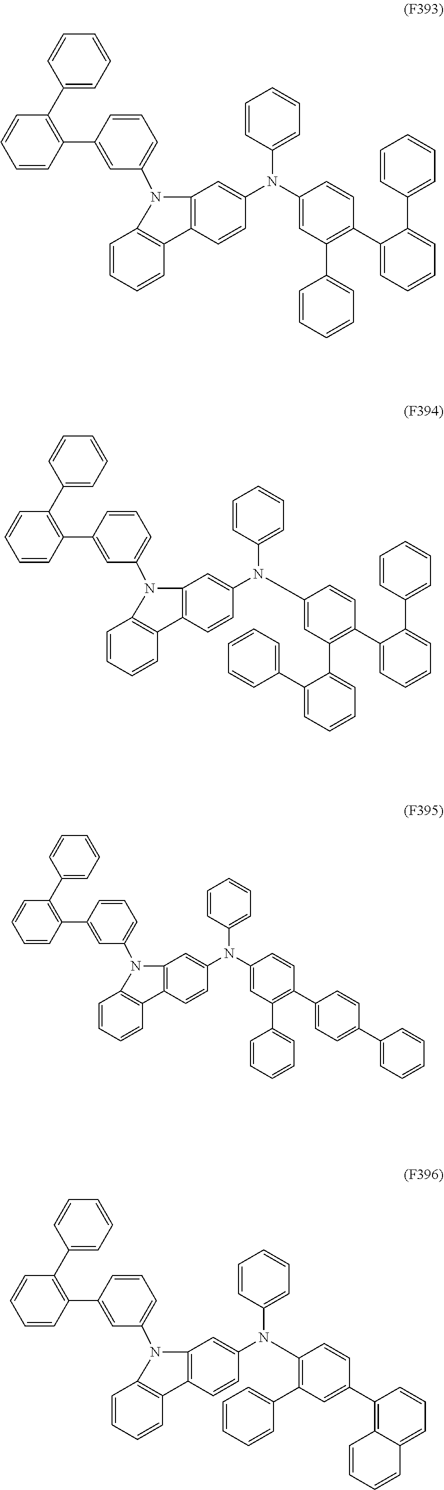 [SMILES: c1ccc(-c2ccccc2-c2cccc(-n3c4ccccc4c4ccc(N(c5ccccc5)c5ccc(-c6ccccc6-c6ccccc6)c(-c6ccccc6)c5)cc43)c2)cc1 ; c1ccc(-c2ccccc2-c2cccc(-n3c4ccccc4c4ccc(N(c5ccccc5)c5ccc(-c6ccccc6-c6ccccc6)c(-c6ccccc6-c6ccccc6)c5)cc43)c2)cc1 ; c1ccc(-c2ccc(-c3ccc(N(c4ccccc4)c4ccc5c6ccccc6n(-c6cccc(-c7ccccc7-c7ccccc7)c6)c5c4)cc3-c3ccccc3)cc2)cc1 ; c1ccc(-c2ccccc2-c2cccc(-n3c4ccccc4c4ccc(N(c5ccccc5)c5ccc(-c6cccc7ccccc67)cc5-c5ccccc5)cc43)c2)cc1]

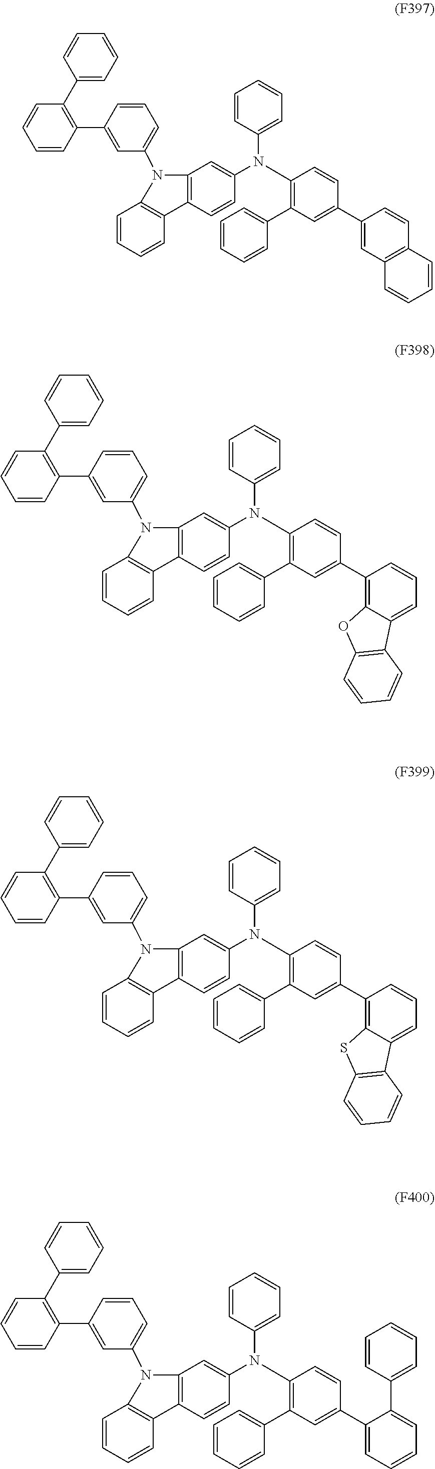 [SMILES: c1ccc(-c2ccccc2-c2cccc(-n3c4ccccc4c4ccc(N(c5ccccc5)c5ccc(-c6ccc7ccccc7c6)cc5-c5ccccc5)cc43)c2)cc1 ; c1ccc(-c2ccccc2-c2cccc(-n3c4ccccc4c4ccc(N(c5ccccc5)c5ccc(-c6cccc7c6oc6ccccc67)cc5-c5ccccc5)cc43)c2)cc1 ; c1ccc(-c2ccccc2-c2cccc(-n3c4ccccc4c4ccc(N(c5ccccc5)c5ccc(-c6cccc7c6sc6ccccc67)cc5-c5ccccc5)cc43)c2)cc1 ; c1ccc(-c2ccccc2-c2cccc(-n3c4ccccc4c4ccc(N(c5ccccc5)c5ccc(-c6ccccc6-c6ccccc6)cc5-c5ccccc5)cc43)c2)cc1]

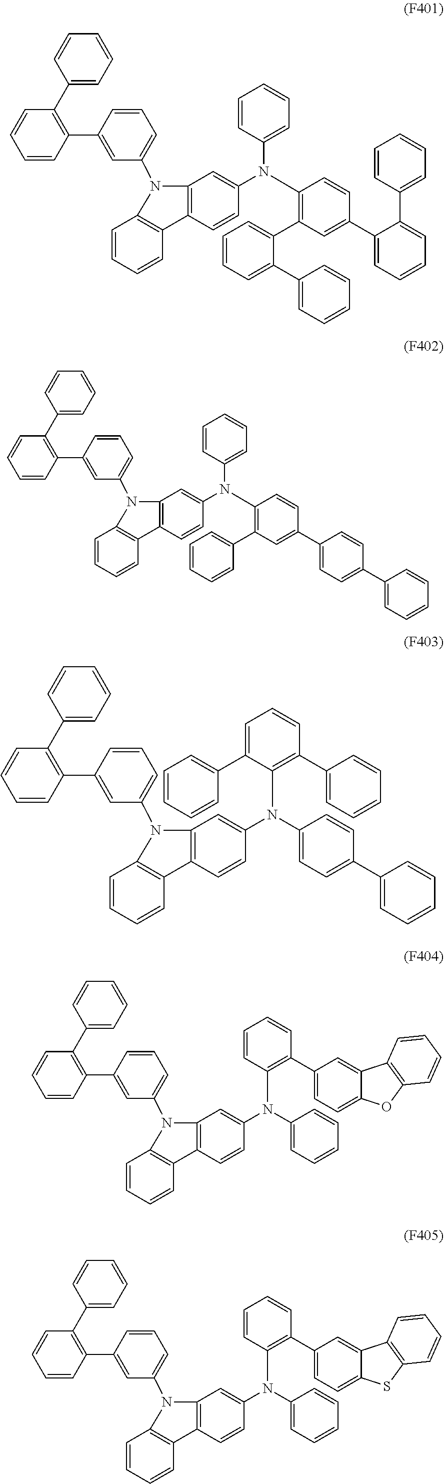

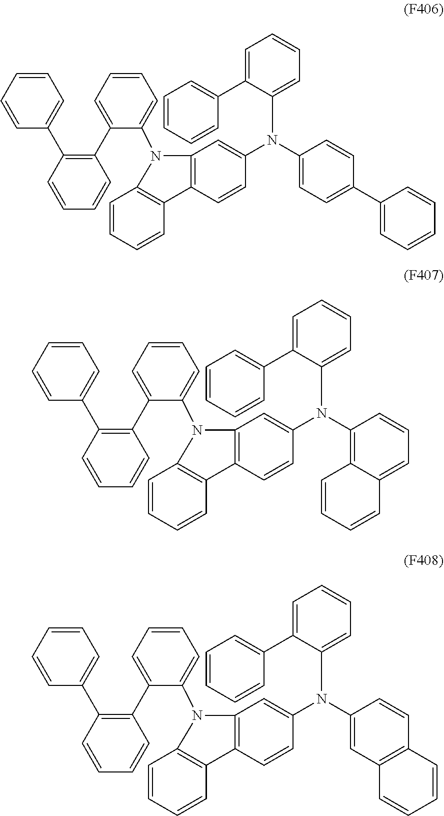 [SMILES: c1ccc(-c2ccc(N(c3ccc4c5ccccc5n(-c5ccccc5-c5ccccc5-c5ccccc5)c4c3)c3ccccc3-c3ccccc3)cc2)cc1 ; c1ccc(-c2ccccc2-c2ccccc2-n2c3ccccc3c3ccc(N(c4ccccc4-c4ccccc4)c4cccc5ccccc45)cc32)cc1 ; c1ccc(-c2ccccc2-c2ccccc2-n2c3ccccc3c3ccc(N(c4ccc5ccccc5c4)c4ccccc4-c4ccccc4)cc32)cc1]

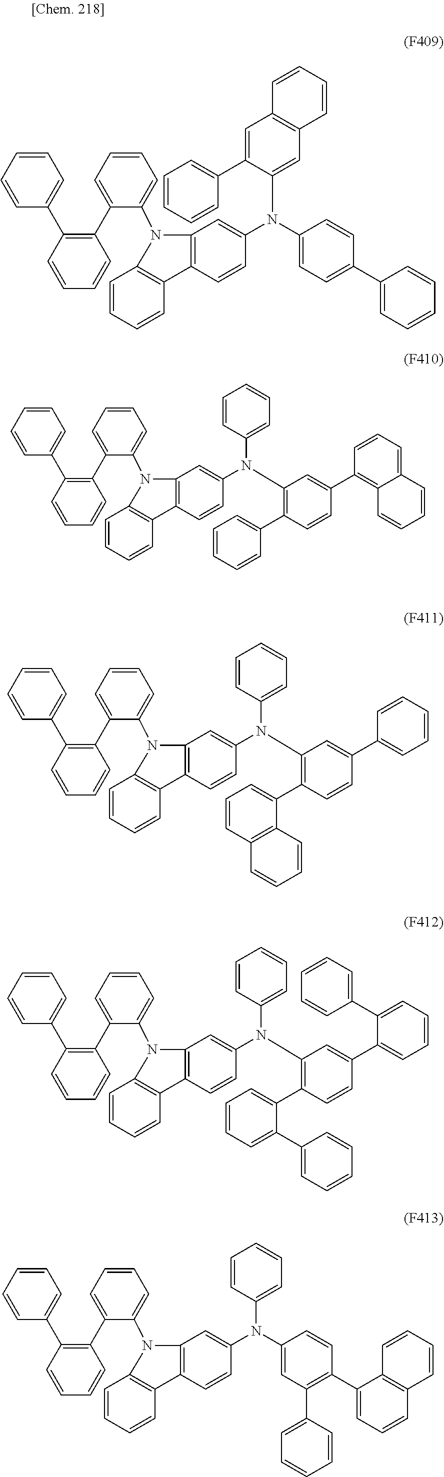

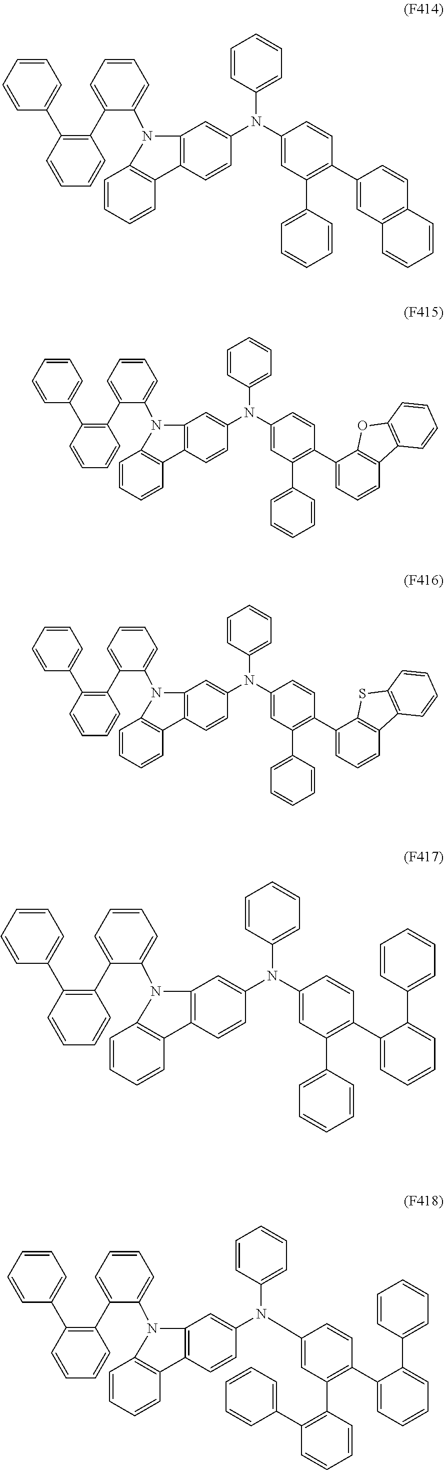

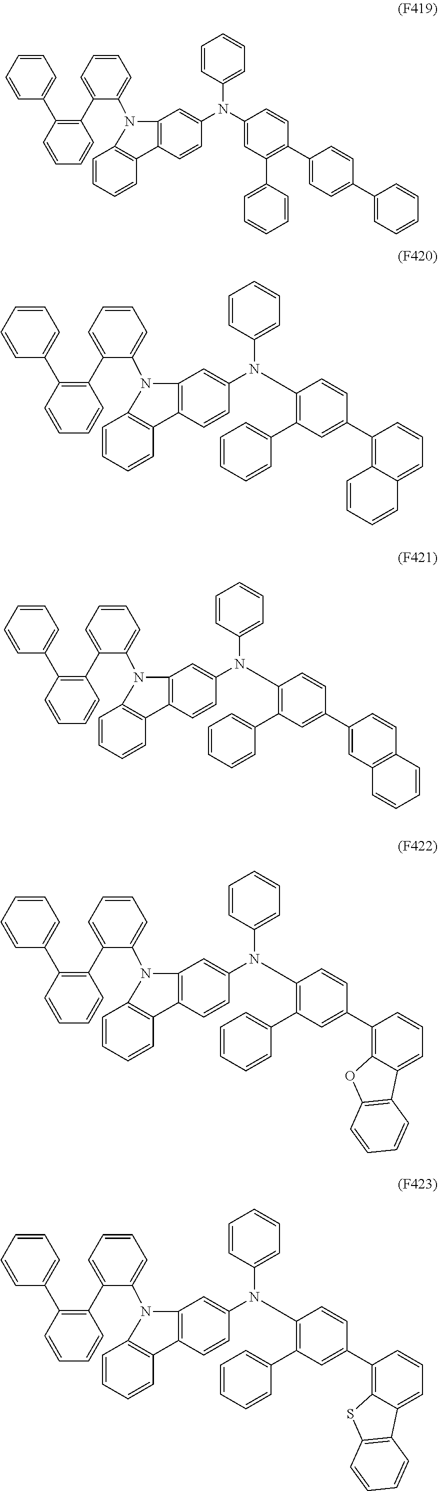

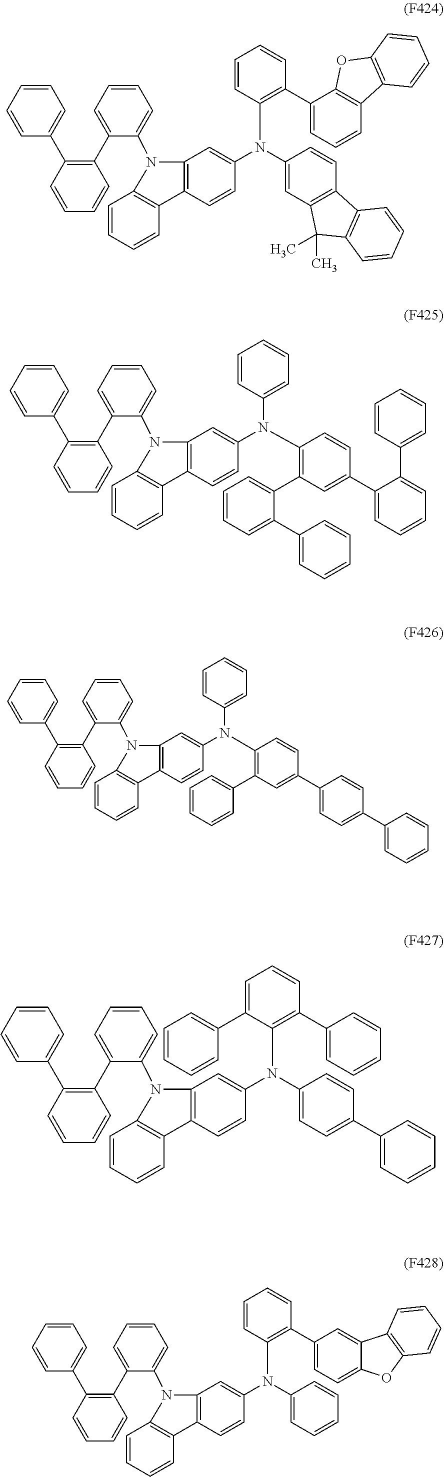

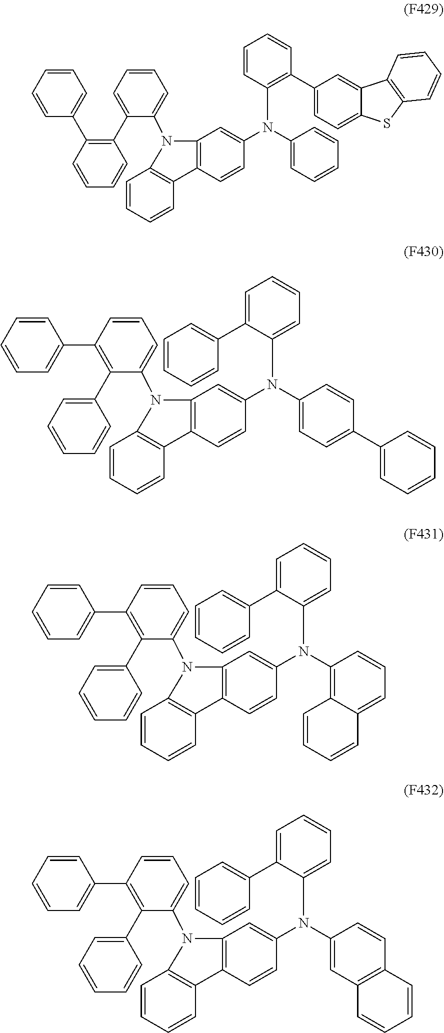 [SMILES: c1ccc(-c2ccccc2-c2ccccc2-n2c3ccccc3c3ccc(N(c4ccccc4)c4ccccc4-c4ccc5sc6ccccc6c5c4)cc32)cc1 ; c1ccc(-c2ccc(N(c3ccc4c5ccccc5n(-c5cccc(-c6ccccc6)c5-c5ccccc5)c4c3)c3ccccc3-c3ccccc3)cc2)cc1 ; c1ccc(-c2ccccc2N(c2ccc3c4ccccc4n(-c4cccc(-c5ccccc5)c4-c4ccccc4)c3c2)c2cccc3ccccc23)cc1 ; c1ccc(-c2ccccc2N(c2ccc3ccccc3c2)c2ccc3c4ccccc4n(-c4cccc(-c5ccccc5)c4-c4ccccc4)c3c2)cc1]

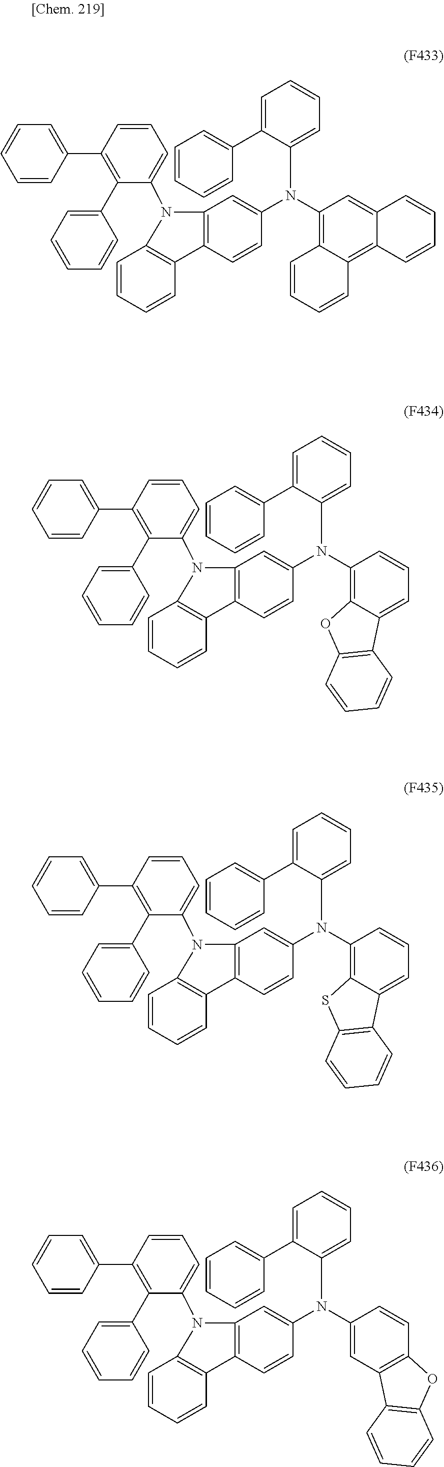 [SMILES: c1ccc(-c2ccccc2N(c2ccc3c4ccccc4n(-c4cccc(-c5ccccc5)c4-c4ccccc4)c3c2)c2cc3ccccc3c3ccccc23)cc1 ; c1ccc(-c2ccccc2N(c2ccc3c4ccccc4n(-c4cccc(-c5ccccc5)c4-c4ccccc4)c3c2)c2cccc3c2oc2ccccc23)cc1 ; c1ccc(-c2ccccc2N(c2ccc3c4ccccc4n(-c4cccc(-c5ccccc5)c4-c4ccccc4)c3c2)c2cccc3c2sc2ccccc23)cc1 ; c1ccc(-c2ccccc2N(c2ccc3oc4ccccc4c3c2)c2ccc3c4ccccc4n(-c4cccc(-c5ccccc5)c4-c4ccccc4)c3c2)cc1]

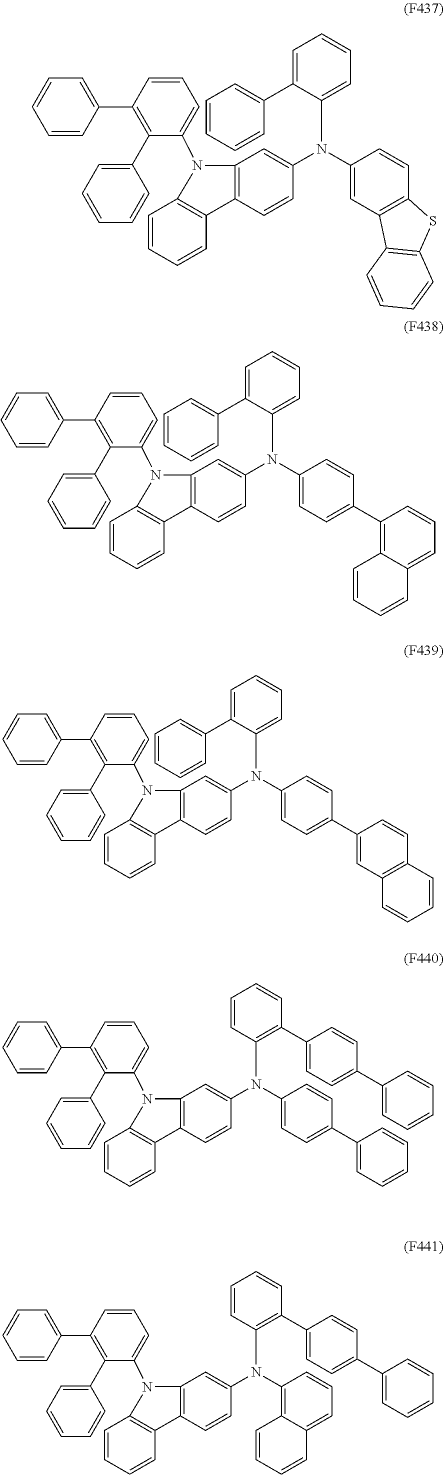 [SMILES: c1ccc(-c2ccccc2N(c2ccc3sc4ccccc4c3c2)c2ccc3c4ccccc4n(-c4cccc(-c5ccccc5)c4-c4ccccc4)c3c2)cc1 ; c1ccc(-c2ccccc2N(c2ccc(-c3cccc4ccccc34)cc2)c2ccc3c4ccccc4n(-c4cccc(-c5ccccc5)c4-c4ccccc4)c3c2)cc1 ; c1ccc(-c2ccccc2N(c2ccc(-c3ccc4ccccc4c3)cc2)c2ccc3c4ccccc4n(-c4cccc(-c5ccccc5)c4-c4ccccc4)c3c2)cc1 ; c1ccc(-c2ccc(-c3ccccc3N(c3ccc(-c4ccccc4)cc3)c3ccc4c5ccccc5n(-c5cccc(-c6ccccc6)c5-c5ccccc5)c4c3)cc2)cc1 ; c1ccc(-c2ccc(-c3ccccc3N(c3ccc4c5ccccc5n(-c5cccc(-c6ccccc6)c5-c5ccccc5)c4c3)c3cccc4ccccc34)cc2)cc1]

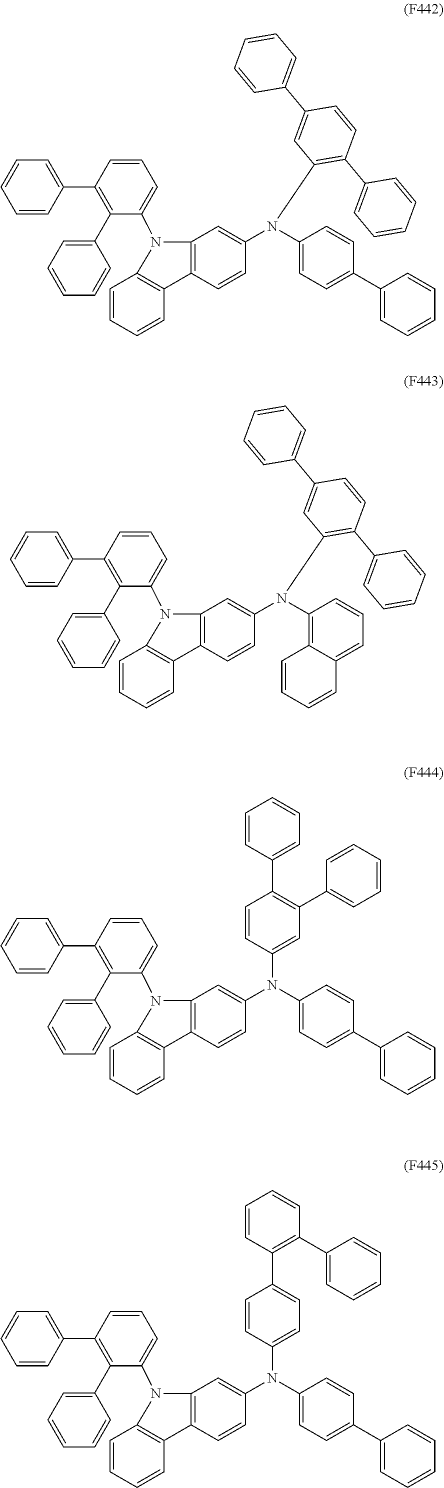 [SMILES: c1ccc(-c2ccc(N(c3ccc4c5ccccc5n(-c5cccc(-c6ccccc6)c5-c5ccccc5)c4c3)c3cc(-c4ccccc4)ccc3-c3ccccc3)cc2)cc1 ; c1ccc(-c2ccc(-c3ccccc3)c(N(c3ccc4c5ccccc5n(-c5cccc(-c6ccccc6)c5-c5ccccc5)c4c3)c3cccc4ccccc34)c2)cc1 ; c1ccc(-c2ccc(N(c3ccc(-c4ccccc4)c(-c4ccccc4)c3)c3ccc4c5ccccc5n(-c5cccc(-c6ccccc6)c5-c5ccccc5)c4c3)cc2)cc1 ; c1ccc(-c2ccc(N(c3ccc(-c4ccccc4-c4ccccc4)cc3)c3ccc4c5ccccc5n(-c5cccc(-c6ccccc6)c5-c5ccccc5)c4c3)cc2)cc1]

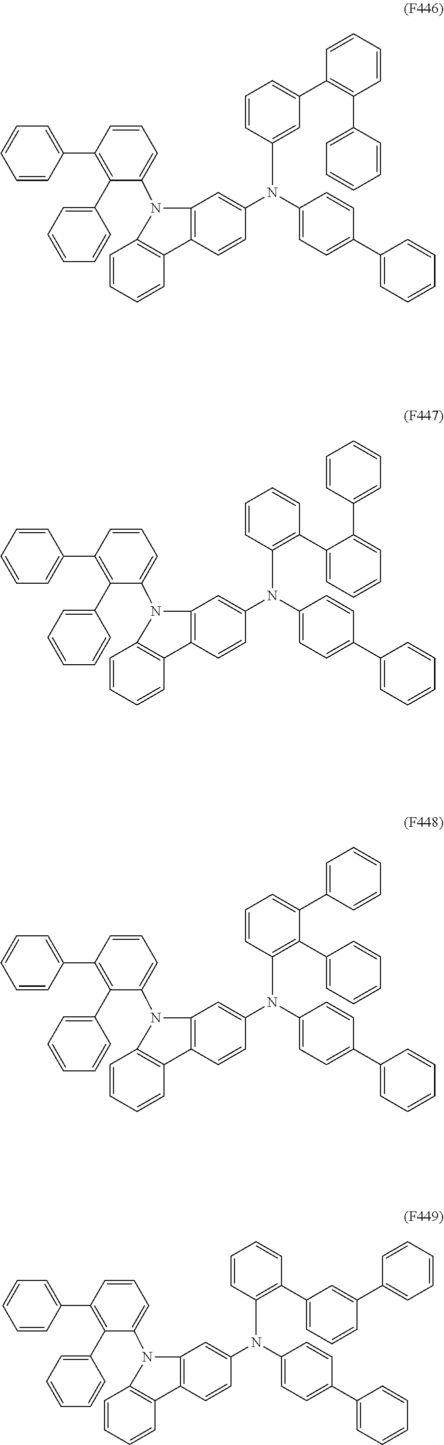 [SMILES: c1ccc(-c2ccc(N(c3cccc(-c4ccccc4-c4ccccc4)c3)c3ccc4c5ccccc5n(-c5cccc(-c6ccccc6)c5-c5ccccc5)c4c3)cc2)cc1 ; c1ccc(-c2ccc(N(c3ccc4c5ccccc5n(-c5cccc(-c6ccccc6)c5-c5ccccc5)c4c3)c3ccccc3-c3ccccc3-c3ccccc3)cc2)cc1 ; c1ccc(-c2ccc(N(c3ccc4c5ccccc5n(-c5cccc(-c6ccccc6)c5-c5ccccc5)c4c3)c3cccc(-c4ccccc4)c3-c3ccccc3)cc2)cc1 ; c1ccc(-c2ccc(N(c3ccc4c5ccccc5n(-c5cccc(-c6ccccc6)c5-c5ccccc5)c4c3)c3ccccc3-c3cccc(-c4ccccc4)c3)cc2)cc1]

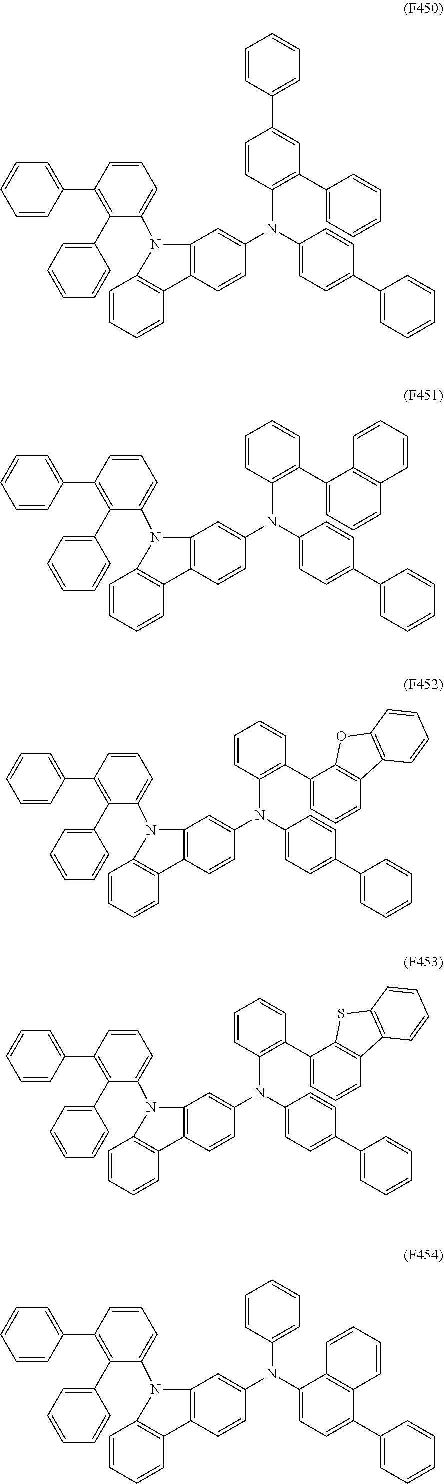

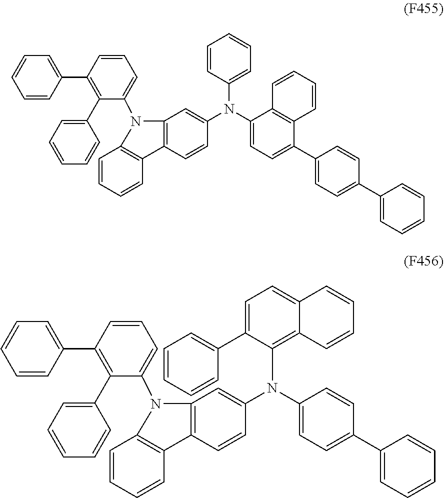 [SMILES: c1ccc(-c2ccc(-c3ccc(N(c4ccccc4)c4ccc5c6ccccc6n(-c6cccc(-c7ccccc7)c6-c6ccccc6)c5c4)c4ccccc34)cc2)cc1 ; c1ccc(-c2ccc(N(c3ccc4c5ccccc5n(-c5cccc(-c6ccccc6)c5-c5ccccc5)c4c3)c3c(-c4ccccc4)ccc4ccccc34)cc2)cc1]

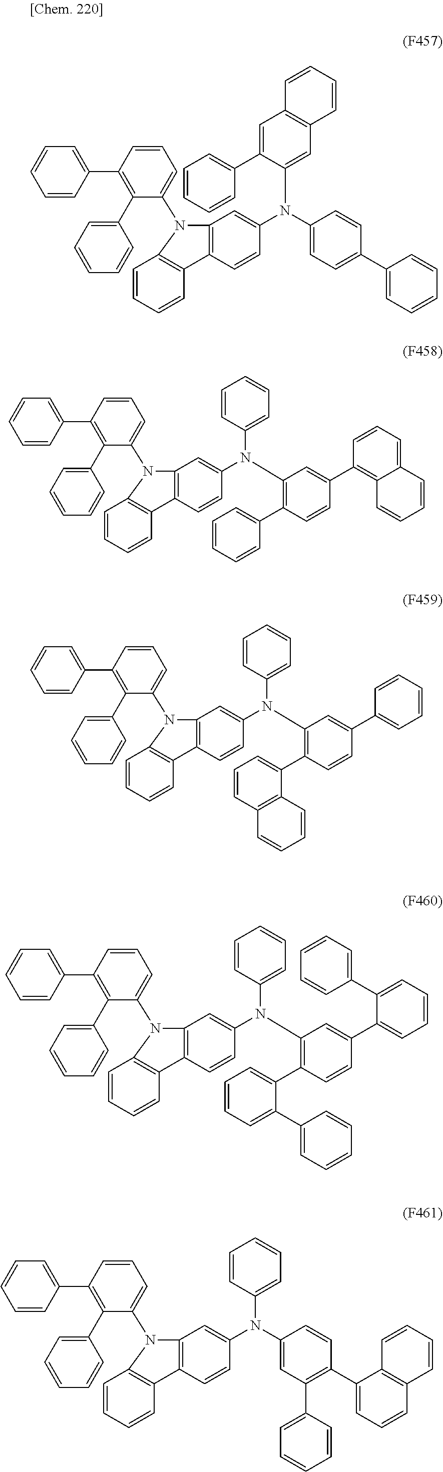

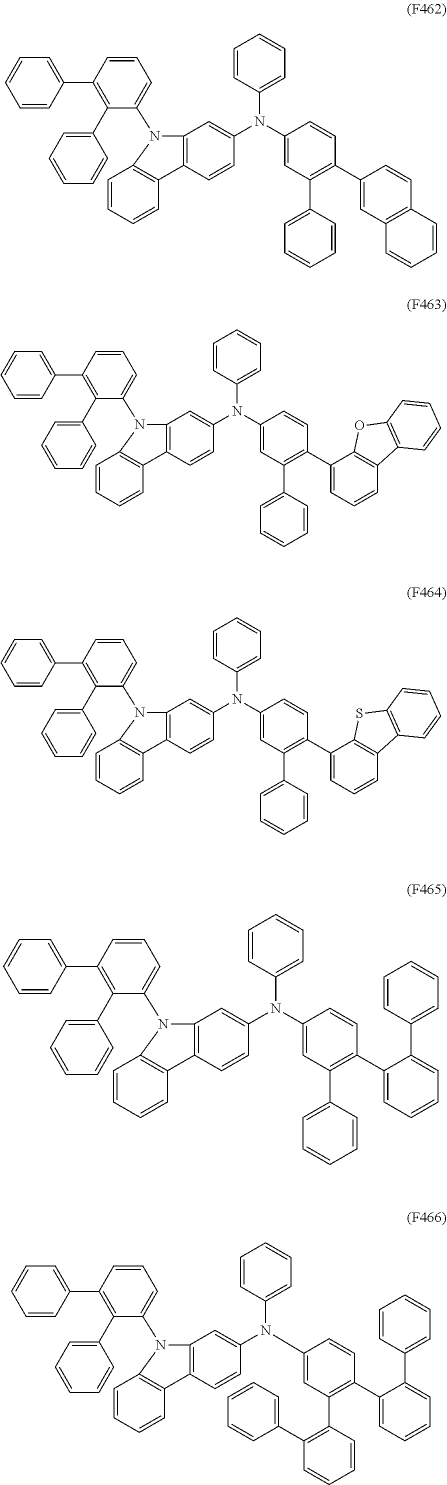

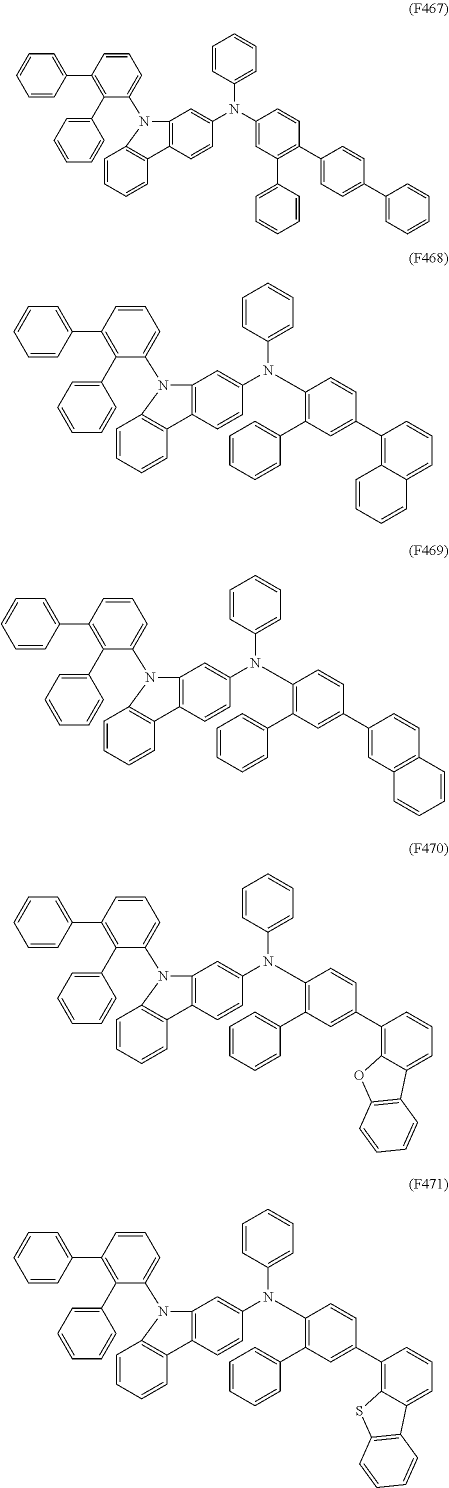

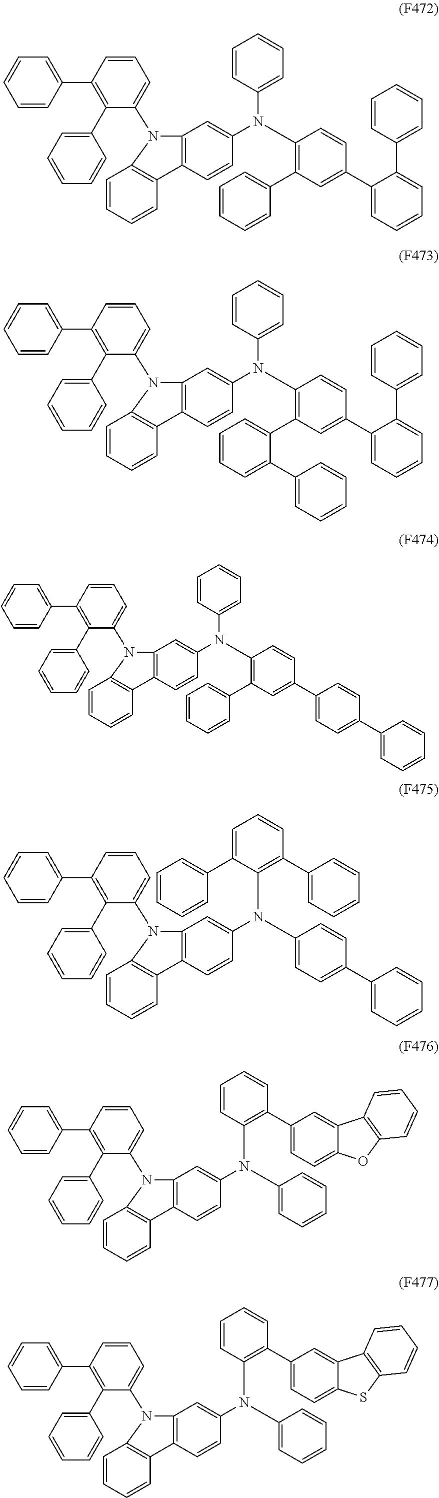

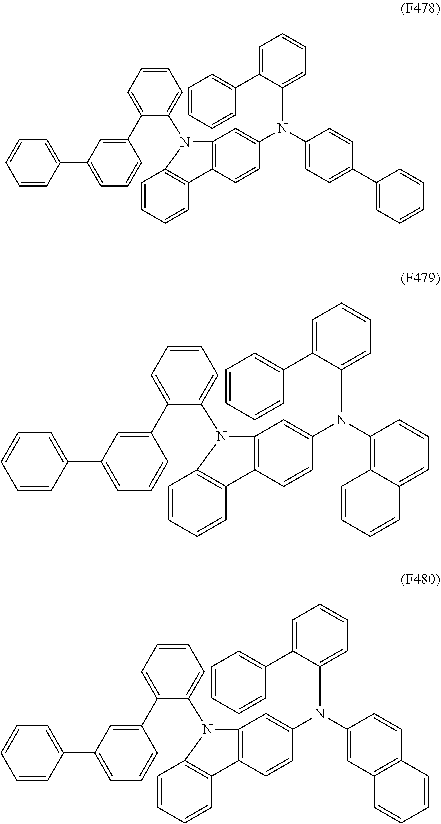 [SMILES: c1ccc(-c2ccc(N(c3ccc4c5ccccc5n(-c5ccccc5-c5cccc(-c6ccccc6)c5)c4c3)c3ccccc3-c3ccccc3)cc2)cc1 ; c1ccc(-c2cccc(-c3ccccc3-n3c4ccccc4c4ccc(N(c5ccccc5-c5ccccc5)c5cccc6ccccc56)cc43)c2)cc1 ; c1ccc(-c2cccc(-c3ccccc3-n3c4ccccc4c4ccc(N(c5ccc6ccccc6c5)c5ccccc5-c5ccccc5)cc43)c2)cc1]

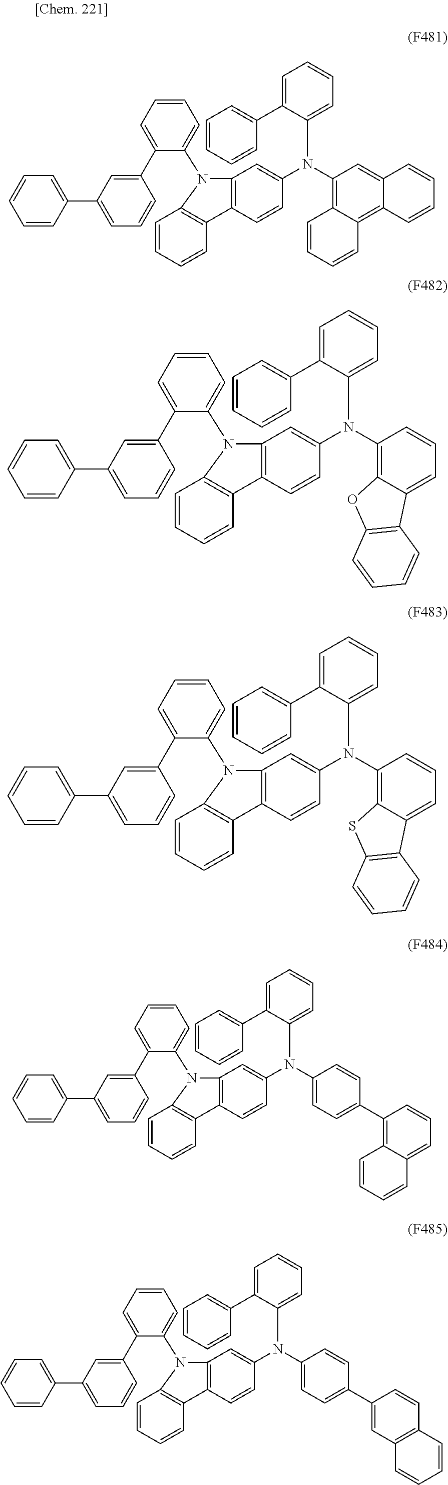 [SMILES: c1ccc(-c2cccc(-c3ccccc3-n3c4ccccc4c4ccc(N(c5ccccc5-c5ccccc5)c5cc6ccccc6c6ccccc56)cc43)c2)cc1 ; c1ccc(-c2cccc(-c3ccccc3-n3c4ccccc4c4ccc(N(c5ccccc5-c5ccccc5)c5cccc6c5oc5ccccc56)cc43)c2)cc1 ; c1ccc(-c2cccc(-c3ccccc3-n3c4ccccc4c4ccc(N(c5ccccc5-c5ccccc5)c5cccc6c5sc5ccccc56)cc43)c2)cc1 ; c1ccc(-c2cccc(-c3ccccc3-n3c4ccccc4c4ccc(N(c5ccc(-c6cccc7ccccc67)cc5)c5ccccc5-c5ccccc5)cc43)c2)cc1 ; c1ccc(-c2cccc(-c3ccccc3-n3c4ccccc4c4ccc(N(c5ccc(-c6ccc7ccccc7c6)cc5)c5ccccc5-c5ccccc5)cc43)c2)cc1]

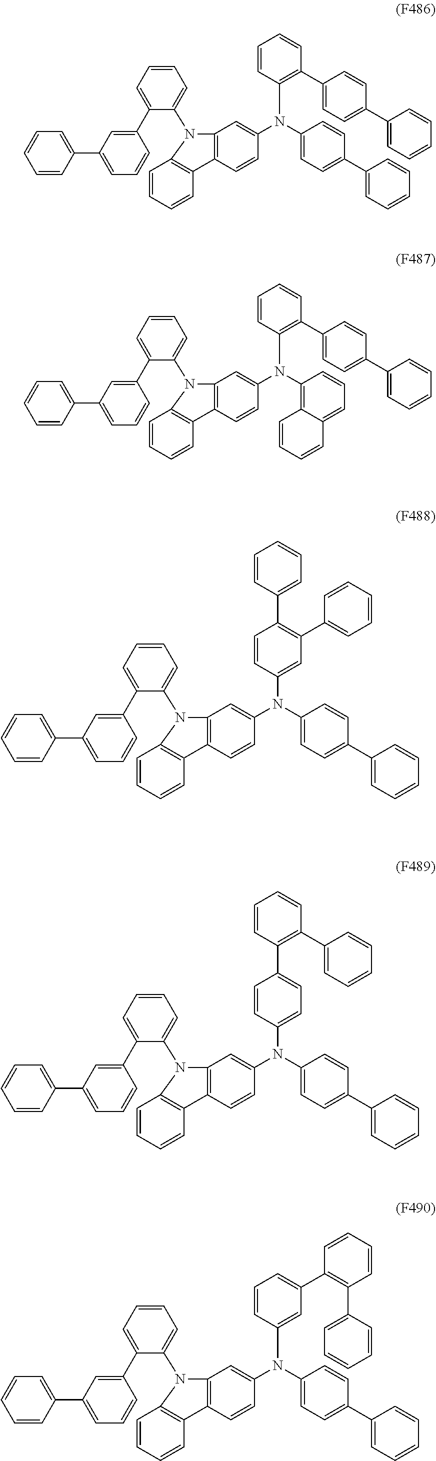

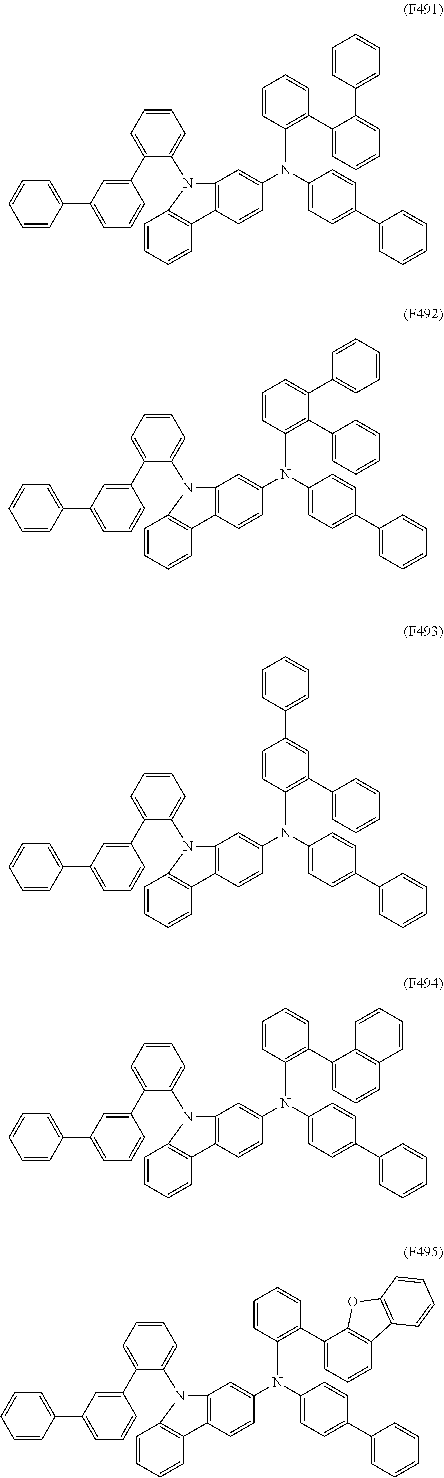

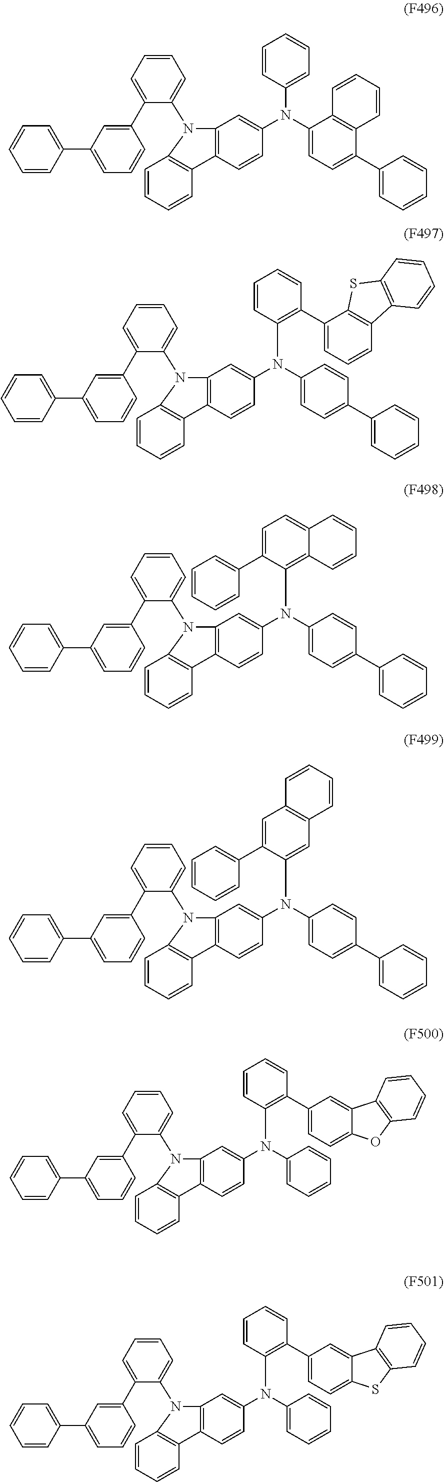

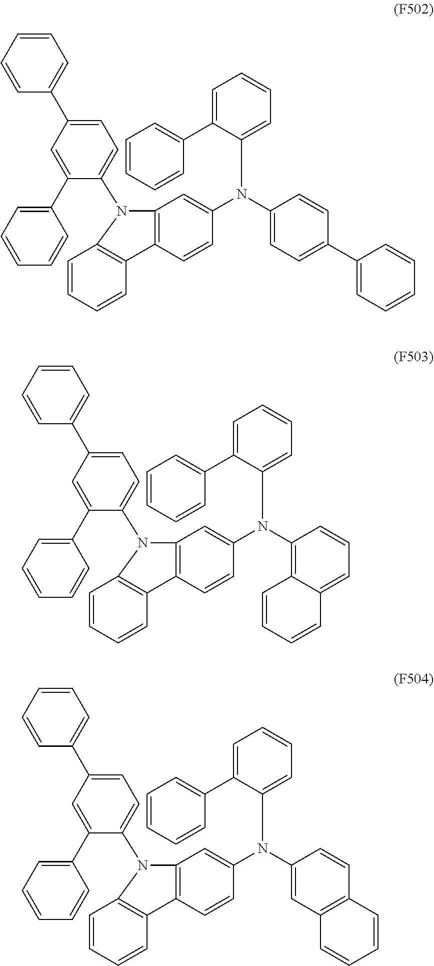 [SMILES: c1ccc(-c2ccc(N(c3ccc4c5ccccc5n(-c5ccc(-c6ccccc6)cc5-c5ccccc5)c4c3)c3ccccc3-c3ccccc3)cc2)cc1 ; c1ccc(-c2ccc(-n3c4ccccc4c4ccc(N(c5ccccc5-c5ccccc5)c5cccc6ccccc56)cc43)c(-c3ccccc3)c2)cc1 ; c1ccc(-c2ccc(-n3c4ccccc4c4ccc(N(c5ccc6ccccc6c5)c5ccccc5-c5ccccc5)cc43)c(-c3ccccc3)c2)cc1]

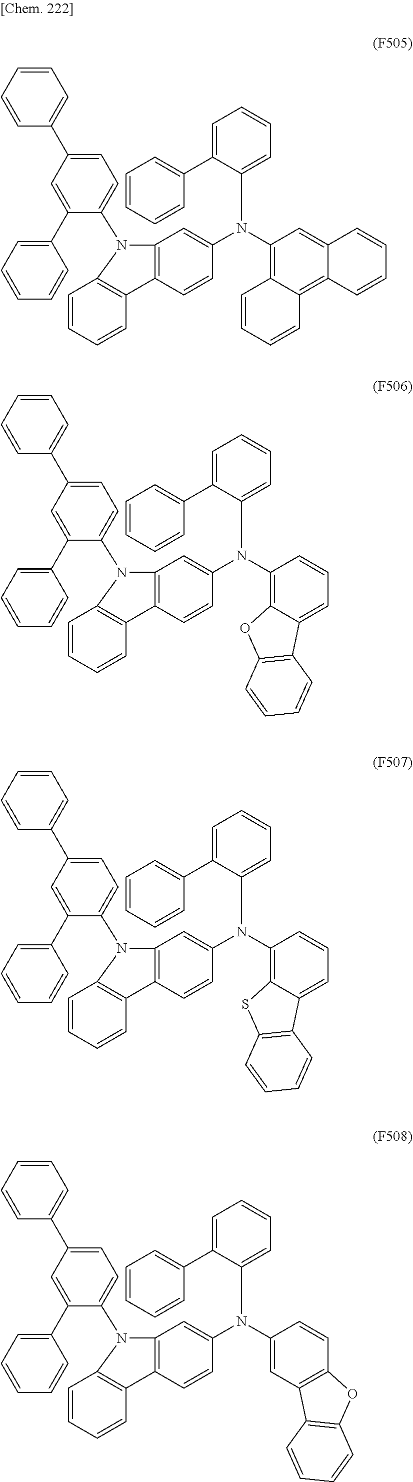 [SMILES: c1ccc(-c2ccc(-n3c4ccccc4c4ccc(N(c5ccccc5-c5ccccc5)c5cc6ccccc6c6ccccc56)cc43)c(-c3ccccc3)c2)cc1 ; c1ccc(-c2ccc(-n3c4ccccc4c4ccc(N(c5ccccc5-c5ccccc5)c5cccc6c5oc5ccccc56)cc43)c(-c3ccccc3)c2)cc1 ; c1ccc(-c2ccc(-n3c4ccccc4c4ccc(N(c5ccccc5-c5ccccc5)c5cccc6c5sc5ccccc56)cc43)c(-c3ccccc3)c2)cc1 ; c1ccc(-c2ccc(-n3c4ccccc4c4ccc(N(c5ccc6oc7ccccc7c6c5)c5ccccc5-c5ccccc5)cc43)c(-c3ccccc3)c2)cc1]

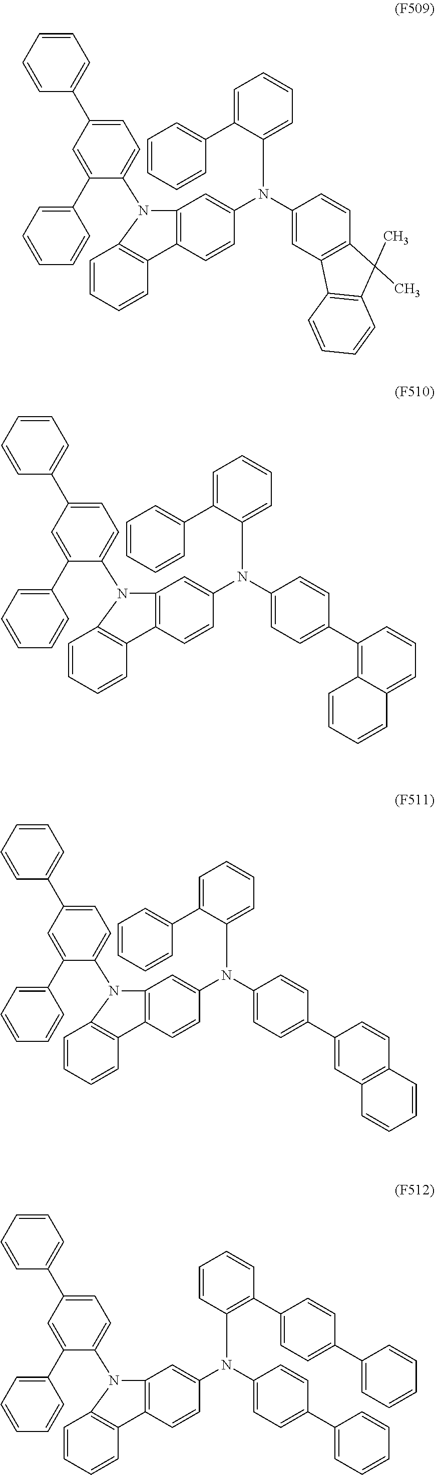 [SMILES: CC1(C)c2ccccc2-c2cc(N(c3ccc4c5ccccc5n(-c5ccc(-c6ccccc6)cc5-c5ccccc5)c4c3)c3ccccc3-c3ccccc3)ccc21 ; c1ccc(-c2ccc(-n3c4ccccc4c4ccc(N(c5ccc(-c6cccc7ccccc67)cc5)c5ccccc5-c5ccccc5)cc43)c(-c3ccccc3)c2)cc1 ; c1ccc(-c2ccc(-n3c4ccccc4c4ccc(N(c5ccc(-c6ccc7ccccc7c6)cc5)c5ccccc5-c5ccccc5)cc43)c(-c3ccccc3)c2)cc1 ; c1ccc(-c2ccc(-c3ccccc3N(c3ccc(-c4ccccc4)cc3)c3ccc4c5ccccc5n(-c5ccc(-c6ccccc6)cc5-c5ccccc5)c4c3)cc2)cc1]

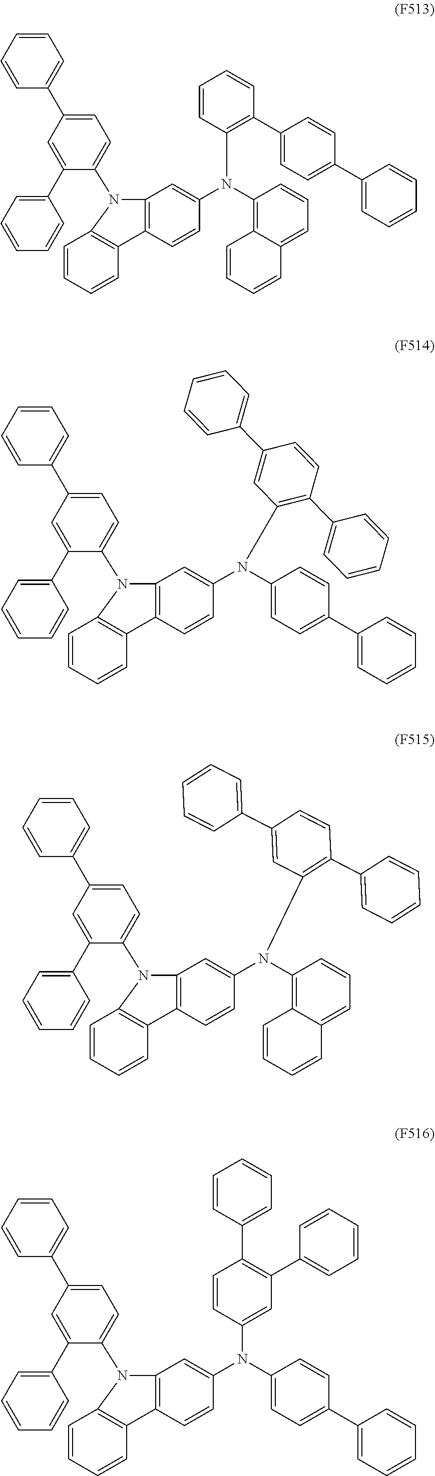 [SMILES: c1ccc(-c2ccc(-c3ccccc3N(c3ccc4c5ccccc5n(-c5ccc(-c6ccccc6)cc5-c5ccccc5)c4c3)c3cccc4ccccc34)cc2)cc1 ; c1ccc(-c2ccc(N(c3ccc4c5ccccc5n(-c5ccc(-c6ccccc6)cc5-c5ccccc5)c4c3)c3cc(-c4ccccc4)ccc3-c3ccccc3)cc2)cc1 ; c1ccc(-c2ccc(-n3c4ccccc4c4ccc(N(c5cc(-c6ccccc6)ccc5-c5ccccc5)c5cccc6ccccc56)cc43)c(-c3ccccc3)c2)cc1 ; c1ccc(-c2ccc(N(c3ccc(-c4ccccc4)c(-c4ccccc4)c3)c3ccc4c5ccccc5n(-c5ccc(-c6ccccc6)cc5-c5ccccc5)c4c3)cc2)cc1]

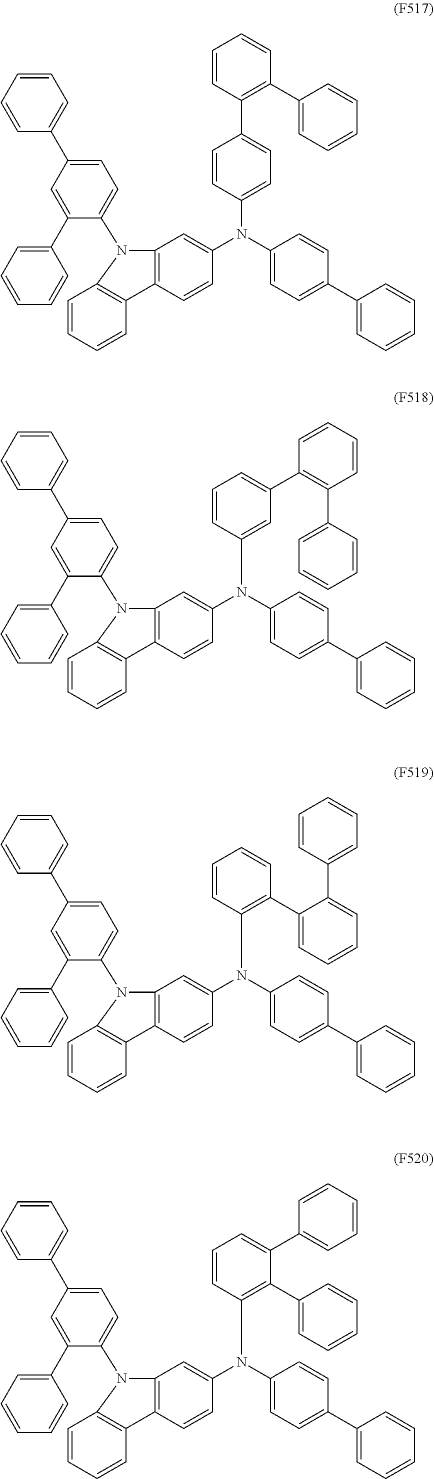 [SMILES: c1ccc(-c2ccc(N(c3ccc(-c4ccccc4-c4ccccc4)cc3)c3ccc4c5ccccc5n(-c5ccc(-c6ccccc6)cc5-c5ccccc5)c4c3)cc2)cc1 ; c1ccc(-c2ccc(N(c3cccc(-c4ccccc4-c4ccccc4)c3)c3ccc4c5ccccc5n(-c5ccc(-c6ccccc6)cc5-c5ccccc5)c4c3)cc2)cc1 ; c1ccc(-c2ccc(N(c3ccc4c5ccccc5n(-c5ccc(-c6ccccc6)cc5-c5ccccc5)c4c3)c3ccccc3-c3ccccc3-c3ccccc3)cc2)cc1 ; c1ccc(-c2ccc(N(c3ccc4c5ccccc5n(-c5ccc(-c6ccccc6)cc5-c5ccccc5)c4c3)c3cccc(-c4ccccc4)c3-c3ccccc3)cc2)cc1]

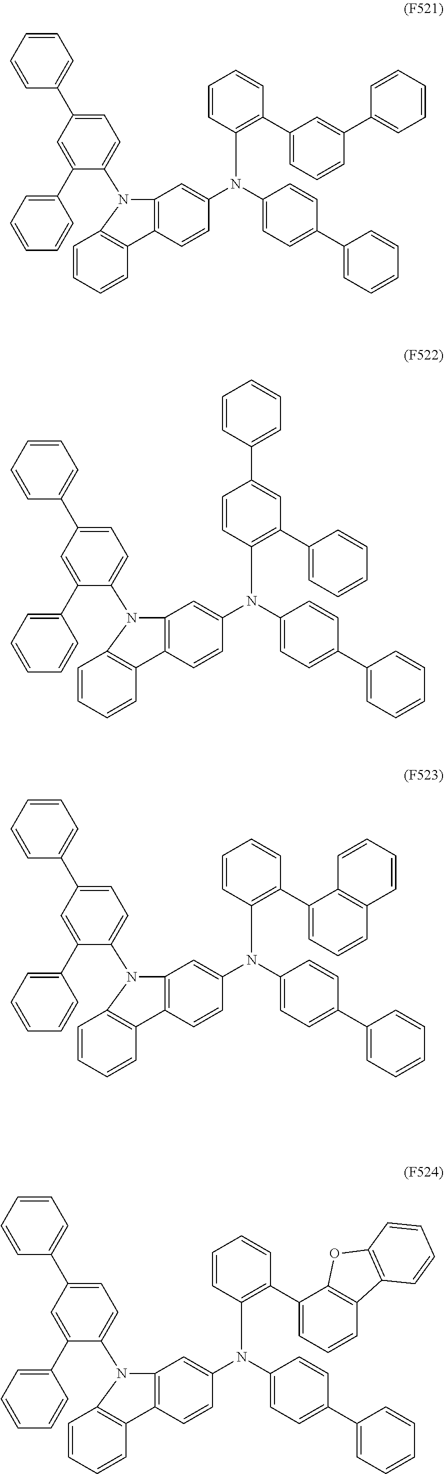 [SMILES: c1ccc(-c2ccc(N(c3ccc4c5ccccc5n(-c5ccc(-c6ccccc6)cc5-c5ccccc5)c4c3)c3ccccc3-c3cccc(-c4ccccc4)c3)cc2)cc1 ; c1ccc(-c2ccc(N(c3ccc4c5ccccc5n(-c5ccc(-c6ccccc6)cc5-c5ccccc5)c4c3)c3ccc(-c4ccccc4)cc3-c3ccccc3)cc2)cc1 ; c1ccc(-c2ccc(N(c3ccc4c5ccccc5n(-c5ccc(-c6ccccc6)cc5-c5ccccc5)c4c3)c3ccccc3-c3cccc4ccccc34)cc2)cc1 ; c1ccc(-c2ccc(N(c3ccc4c5ccccc5n(-c5ccc(-c6ccccc6)cc5-c5ccccc5)c4c3)c3ccccc3-c3cccc4c3oc3ccccc34)cc2)cc1]

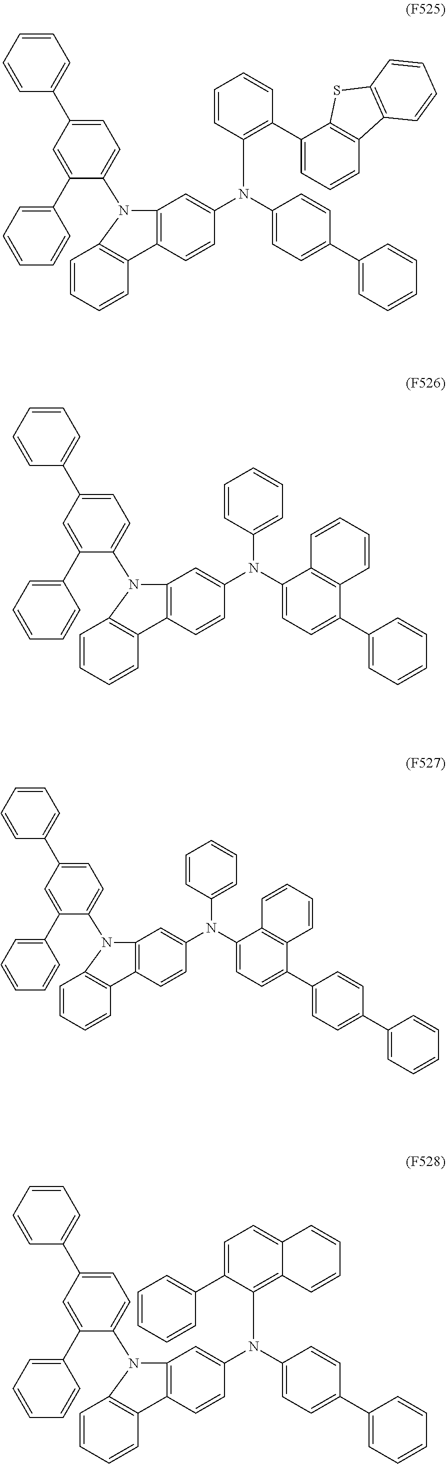 [SMILES: c1ccc(-c2ccc(N(c3ccc4c5ccccc5n(-c5ccc(-c6ccccc6)cc5-c5ccccc5)c4c3)c3ccccc3-c3cccc4c3sc3ccccc34)cc2)cc1 ; c1ccc(-c2ccc(-n3c4ccccc4c4ccc(N(c5ccccc5)c5ccc(-c6ccccc6)c6ccccc56)cc43)c(-c3ccccc3)c2)cc1 ; c1ccc(-c2ccc(-c3ccc(N(c4ccccc4)c4ccc5c6ccccc6n(-c6ccc(-c7ccccc7)cc6-c6ccccc6)c5c4)c4ccccc34)cc2)cc1 ; c1ccc(-c2ccc(N(c3ccc4c5ccccc5n(-c5ccc(-c6ccccc6)cc5-c5ccccc5)c4c3)c3c(-c4ccccc4)ccc4ccccc34)cc2)cc1]

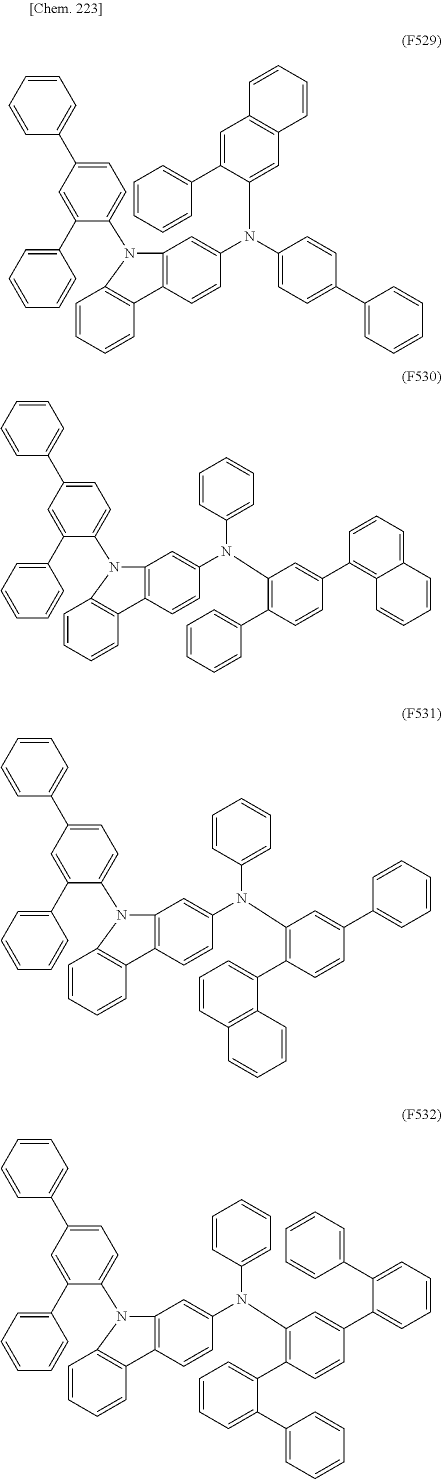 [SMILES: c1ccc(-c2ccc(N(c3ccc4c5ccccc5n(-c5ccc(-c6ccccc6)cc5-c5ccccc5)c4c3)c3cc4ccccc4cc3-c3ccccc3)cc2)cc1 ; c1ccc(-c2ccc(-n3c4ccccc4c4ccc(N(c5ccccc5)c5cc(-c6cccc7ccccc67)ccc5-c5ccccc5)cc43)c(-c3ccccc3)c2)cc1 ; c1ccc(-c2ccc(-n3c4ccccc4c4ccc(N(c5ccccc5)c5cc(-c6ccccc6)ccc5-c5cccc6ccccc56)cc43)c(-c3ccccc3)c2)cc1 ; c1ccc(-c2ccc(-n3c4ccccc4c4ccc(N(c5ccccc5)c5cc(-c6ccccc6-c6ccccc6)ccc5-c5ccccc5-c5ccccc5)cc43)c(-c3ccccc3)c2)cc1]

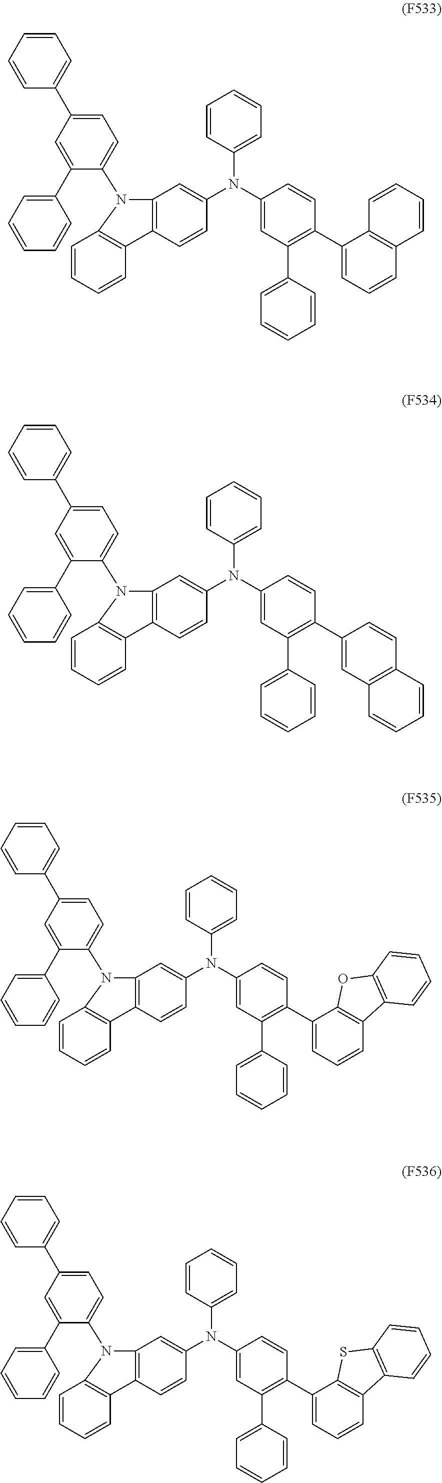 [SMILES: c1ccc(-c2ccc(-n3c4ccccc4c4ccc(N(c5ccccc5)c5ccc(-c6cccc7ccccc67)c(-c6ccccc6)c5)cc43)c(-c3ccccc3)c2)cc1 ; c1ccc(-c2ccc(-n3c4ccccc4c4ccc(N(c5ccccc5)c5ccc(-c6ccc7ccccc7c6)c(-c6ccccc6)c5)cc43)c(-c3ccccc3)c2)cc1 ; c1ccc(-c2ccc(-n3c4ccccc4c4ccc(N(c5ccccc5)c5ccc(-c6cccc7c6oc6ccccc67)c(-c6ccccc6)c5)cc43)c(-c3ccccc3)c2)cc1 ; c1ccc(-c2ccc(-n3c4ccccc4c4ccc(N(c5ccccc5)c5ccc(-c6cccc7c6sc6ccccc67)c(-c6ccccc6)c5)cc43)c(-c3ccccc3)c2)cc1]

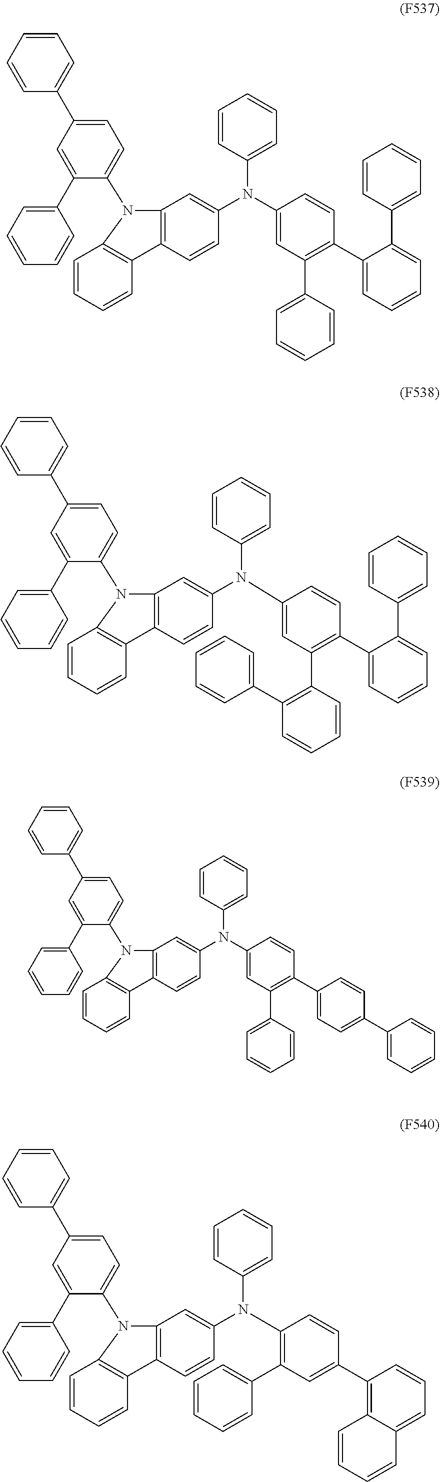 [SMILES: c1ccc(-c2ccc(-n3c4ccccc4c4ccc(N(c5ccccc5)c5ccc(-c6ccccc6-c6ccccc6)c(-c6ccccc6)c5)cc43)c(-c3ccccc3)c2)cc1 ; c1ccc(-c2ccc(-n3c4ccccc4c4ccc(N(c5ccccc5)c5ccc(-c6ccccc6-c6ccccc6)c(-c6ccccc6-c6ccccc6)c5)cc43)c(-c3ccccc3)c2)cc1 ; c1ccc(-c2ccc(-c3ccc(N(c4ccccc4)c4ccc5c6ccccc6n(-c6ccc(-c7ccccc7)cc6-c6ccccc6)c5c4)cc3-c3ccccc3)cc2)cc1 ; c1ccc(-c2ccc(-n3c4ccccc4c4ccc(N(c5ccccc5)c5ccc(-c6cccc7ccccc67)cc5-c5ccccc5)cc43)c(-c3ccccc3)c2)cc1]

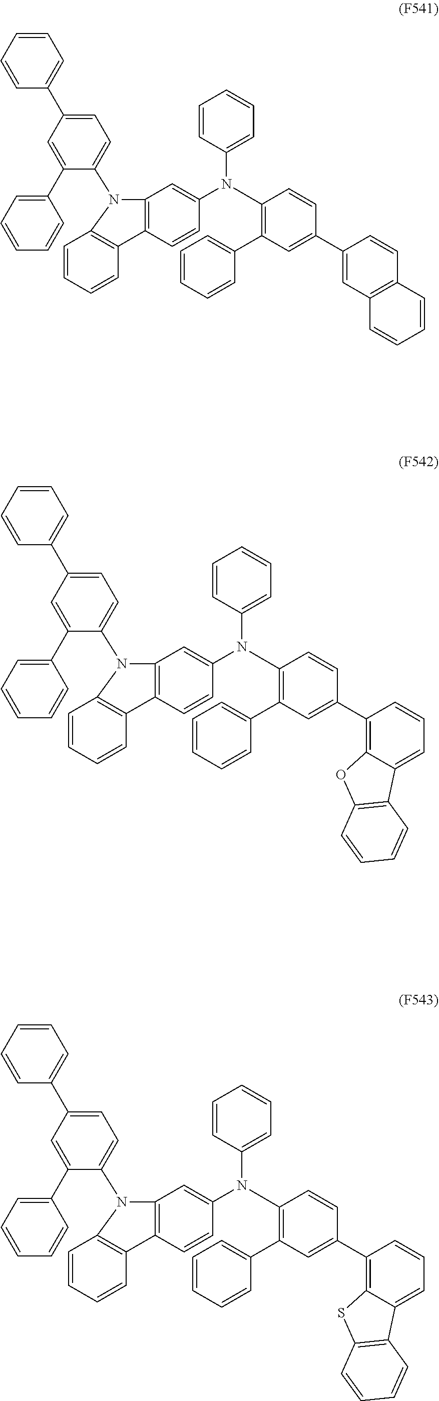 [SMILES: c1ccc(-c2ccc(-n3c4ccccc4c4ccc(N(c5ccccc5)c5ccc(-c6ccc7ccccc7c6)cc5-c5ccccc5)cc43)c(-c3ccccc3)c2)cc1 ; c1ccc(-c2ccc(-n3c4ccccc4c4ccc(N(c5ccccc5)c5ccc(-c6cccc7c6oc6ccccc67)cc5-c5ccccc5)cc43)c(-c3ccccc3)c2)cc1 ; c1ccc(-c2ccc(-n3c4ccccc4c4ccc(N(c5ccccc5)c5ccc(-c6cccc7c6sc6ccccc67)cc5-c5ccccc5)cc43)c(-c3ccccc3)c2)cc1]

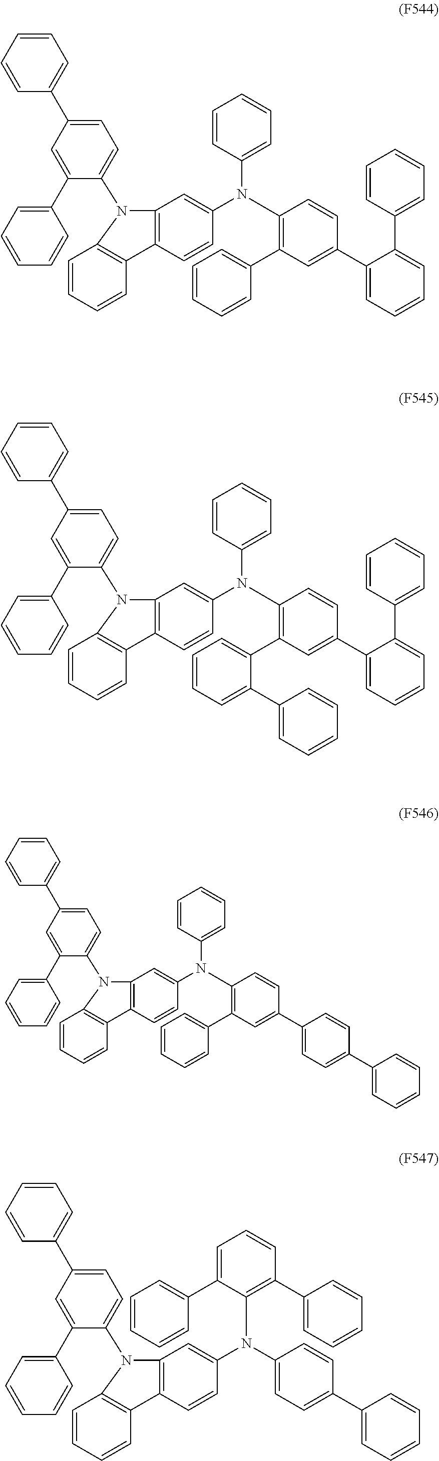

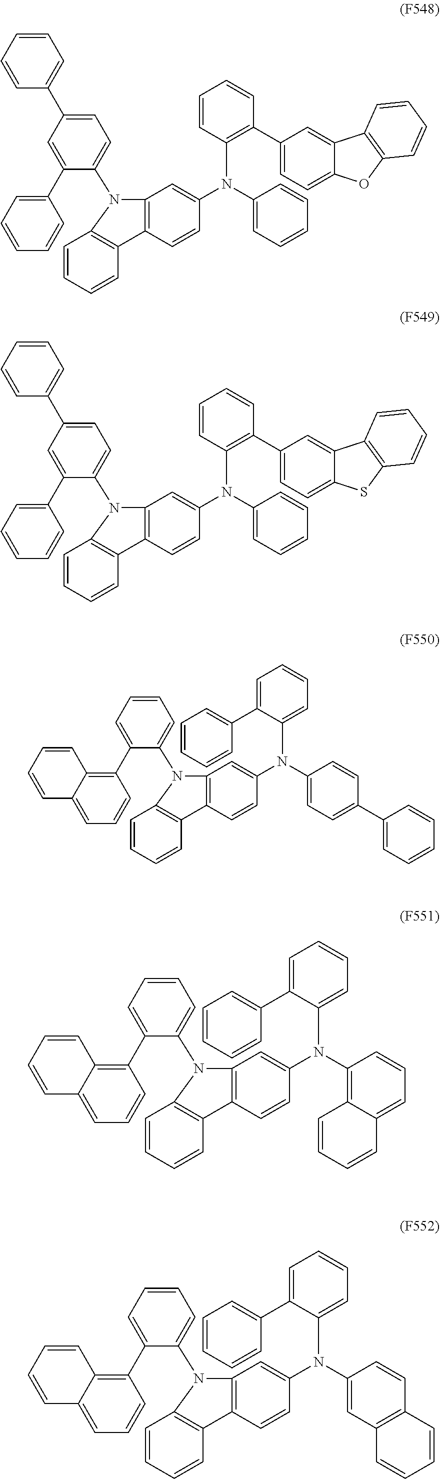

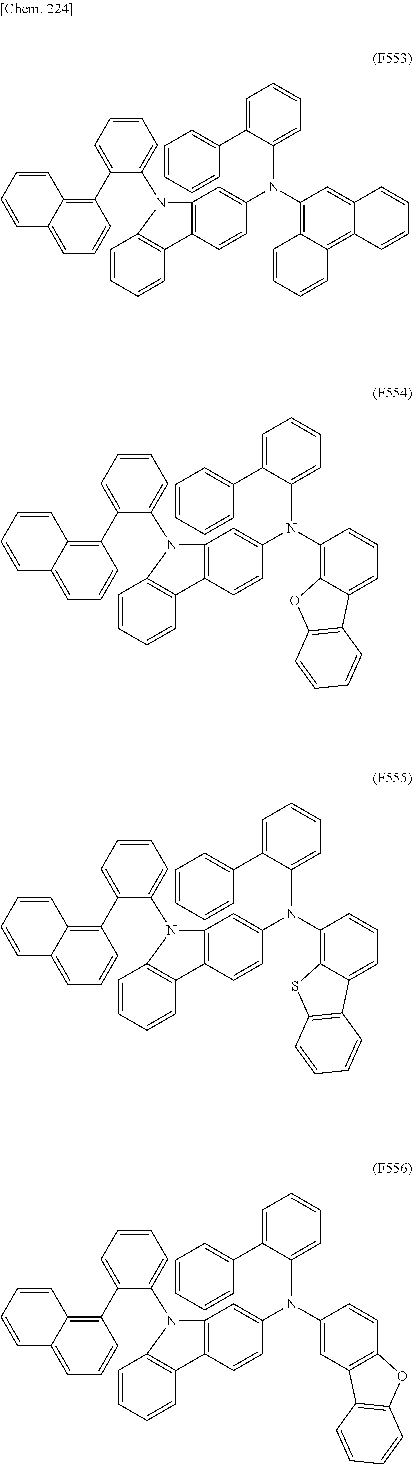 [SMILES: c1ccc(-c2ccccc2N(c2ccc3c4ccccc4n(-c4ccccc4-c4cccc5ccccc45)c3c2)c2cc3ccccc3c3ccccc23)cc1 ; c1ccc(-c2ccccc2N(c2ccc3c4ccccc4n(-c4ccccc4-c4cccc5ccccc45)c3c2)c2cccc3c2oc2ccccc23)cc1 ; c1ccc(-c2ccccc2N(c2ccc3c4ccccc4n(-c4ccccc4-c4cccc5ccccc45)c3c2)c2cccc3c2sc2ccccc23)cc1 ; c1ccc(-c2ccccc2N(c2ccc3oc4ccccc4c3c2)c2ccc3c4ccccc4n(-c4ccccc4-c4cccc5ccccc45)c3c2)cc1]

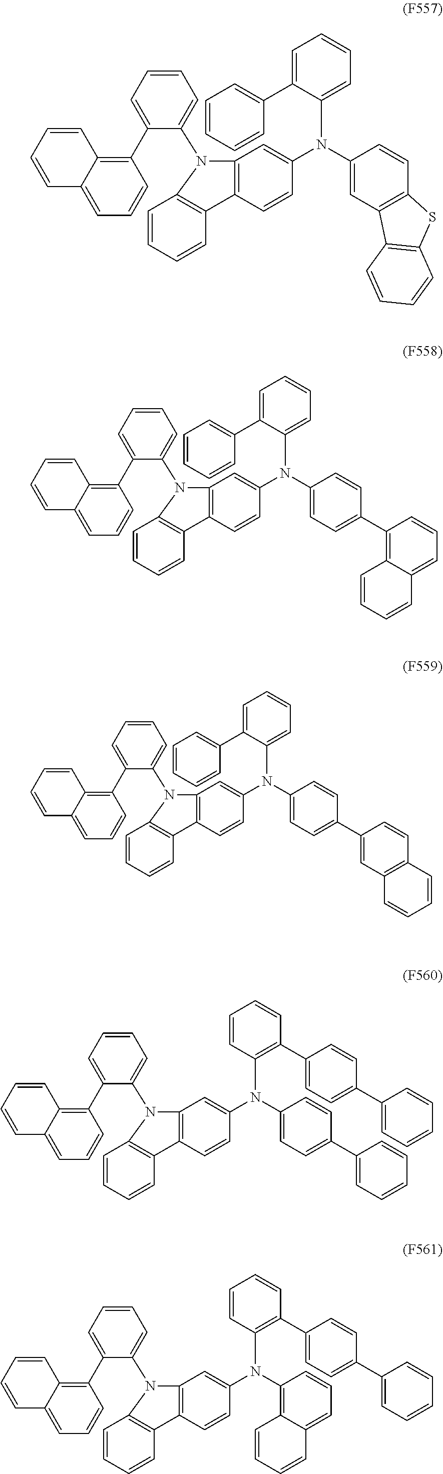

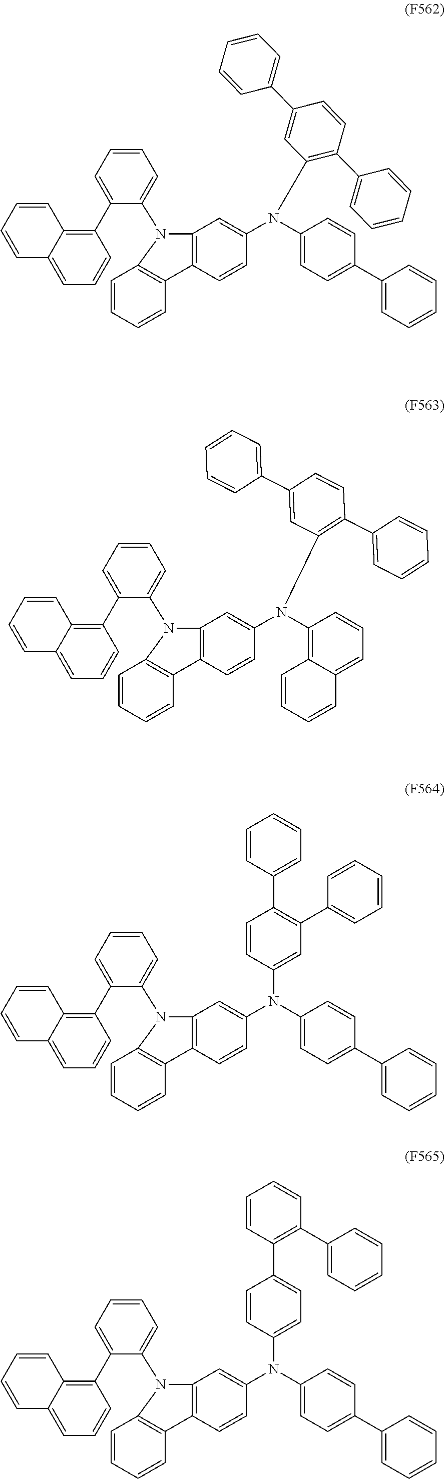 [SMILES: c1ccc(-c2ccc(N(c3ccc4c5ccccc5n(-c5ccccc5-c5cccc6ccccc56)c4c3)c3cc(-c4ccccc4)ccc3-c3ccccc3)cc2)cc1 ; c1ccc(-c2ccc(-c3ccccc3)c(N(c3ccc4c5ccccc5n(-c5ccccc5-c5cccc6ccccc56)c4c3)c3cccc4ccccc34)c2)cc1 ; c1ccc(-c2ccc(N(c3ccc(-c4ccccc4)c(-c4ccccc4)c3)c3ccc4c5ccccc5n(-c5ccccc5-c5cccc6ccccc56)c4c3)cc2)cc1 ; c1ccc(-c2ccc(N(c3ccc(-c4ccccc4-c4ccccc4)cc3)c3ccc4c5ccccc5n(-c5ccccc5-c5cccc6ccccc56)c4c3)cc2)cc1]

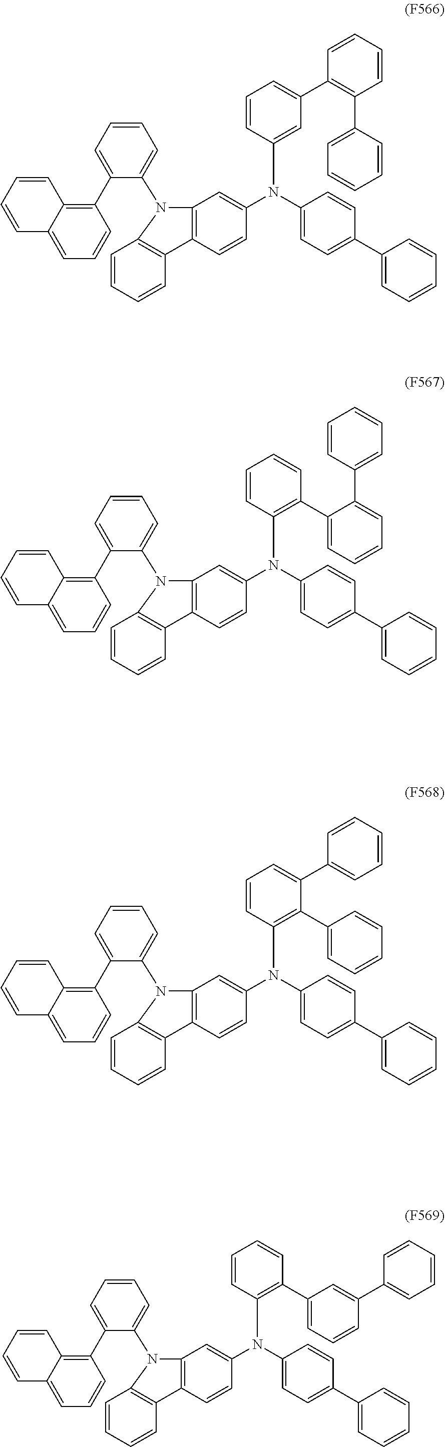 [SMILES: c1ccc(-c2ccc(N(c3cccc(-c4ccccc4-c4ccccc4)c3)c3ccc4c5ccccc5n(-c5ccccc5-c5cccc6ccccc56)c4c3)cc2)cc1 ; c1ccc(-c2ccc(N(c3ccc4c5ccccc5n(-c5ccccc5-c5cccc6ccccc56)c4c3)c3ccccc3-c3ccccc3-c3ccccc3)cc2)cc1 ; c1ccc(-c2ccc(N(c3ccc4c5ccccc5n(-c5ccccc5-c5cccc6ccccc56)c4c3)c3cccc(-c4ccccc4)c3-c3ccccc3)cc2)cc1 ; c1ccc(-c2ccc(N(c3ccc4c5ccccc5n(-c5ccccc5-c5cccc6ccccc56)c4c3)c3ccccc3-c3cccc(-c4ccccc4)c3)cc2)cc1]

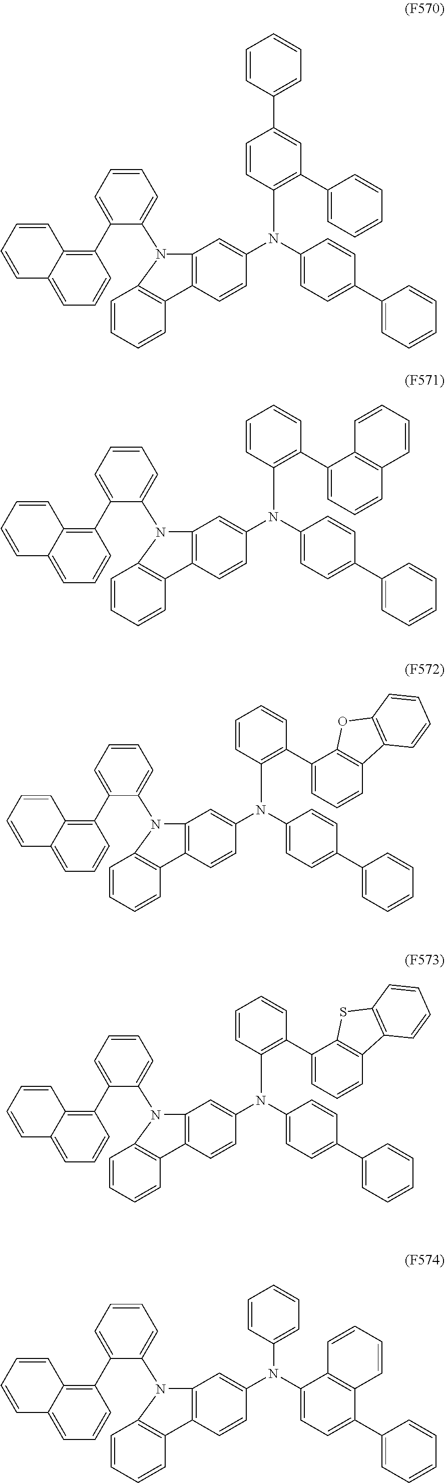 [SMILES: c1ccc(-c2ccc(N(c3ccc4c5ccccc5n(-c5ccccc5-c5cccc6ccccc56)c4c3)c3ccc(-c4ccccc4)cc3-c3ccccc3)cc2)cc1 ; c1ccc(-c2ccc(N(c3ccc4c5ccccc5n(-c5ccccc5-c5cccc6ccccc56)c4c3)c3ccccc3-c3cccc4ccccc34)cc2)cc1 ; c1ccc(-c2ccc(N(c3ccc4c5ccccc5n(-c5ccccc5-c5cccc6ccccc56)c4c3)c3ccccc3-c3cccc4c3oc3ccccc34)cc2)cc1 ; c1ccc(-c2ccc(N(c3ccc4c5ccccc5n(-c5ccccc5-c5cccc6ccccc56)c4c3)c3ccccc3-c3cccc4c3sc3ccccc34)cc2)cc1 ; c1ccc(-c2ccc(N(c3ccccc3)c3ccc4c5ccccc5n(-c5ccccc5-c5cccc6ccccc56)c4c3)c3ccccc23)cc1]

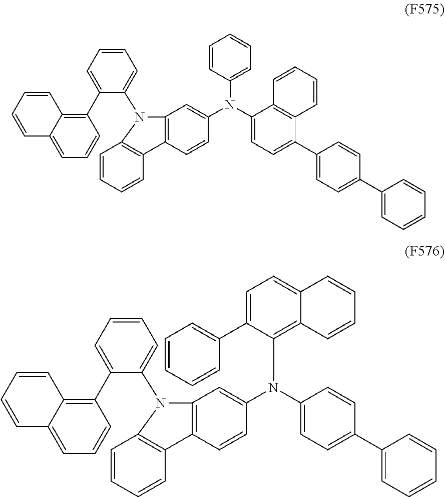 [SMILES: c1ccc(-c2ccc(-c3ccc(N(c4ccccc4)c4ccc5c6ccccc6n(-c6ccccc6-c6cccc7ccccc67)c5c4)c4ccccc34)cc2)cc1 ; c1ccc(-c2ccc(N(c3ccc4c5ccccc5n(-c5ccccc5-c5cccc6ccccc56)c4c3)c3c(-c4ccccc4)ccc4ccccc34)cc2)cc1]

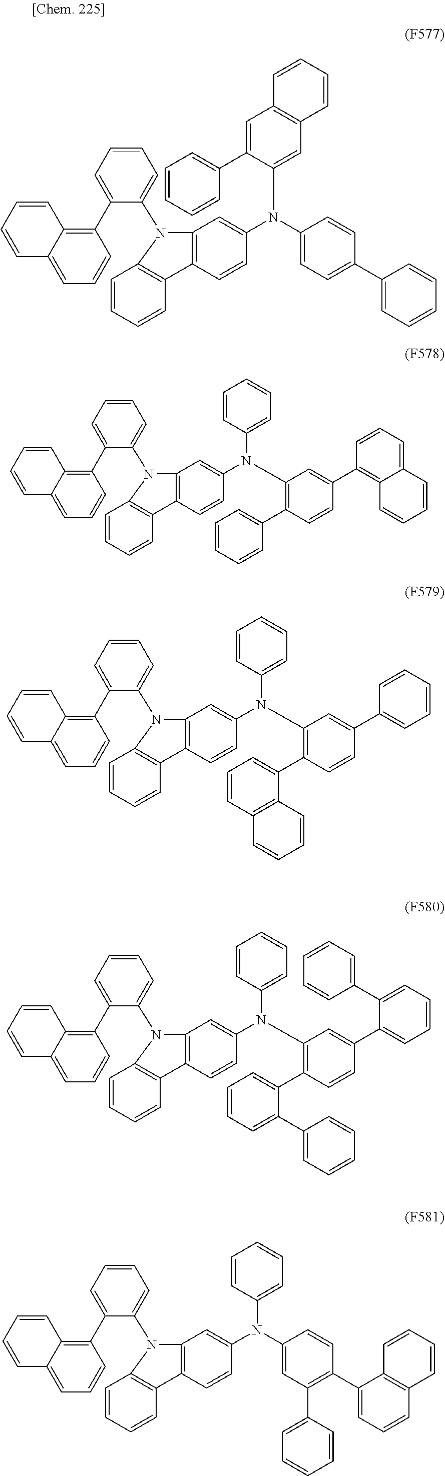 [SMILES: c1ccc(-c2ccc(N(c3ccc4c5ccccc5n(-c5ccccc5-c5cccc6ccccc56)c4c3)c3cc4ccccc4cc3-c3ccccc3)cc2)cc1 ; c1ccc(-c2ccc(-c3cccc4ccccc34)cc2N(c2ccccc2)c2ccc3c4ccccc4n(-c4ccccc4-c4cccc5ccccc45)c3c2)cc1 ; c1ccc(-c2ccc(-c3cccc4ccccc34)c(N(c3ccccc3)c3ccc4c5ccccc5n(-c5ccccc5-c5cccc6ccccc56)c4c3)c2)cc1 ; c1ccc(-c2ccccc2-c2ccc(-c3ccccc3-c3ccccc3)c(N(c3ccccc3)c3ccc4c5ccccc5n(-c5ccccc5-c5cccc6ccccc56)c4c3)c2)cc1 ; c1ccc(-c2cc(N(c3ccccc3)c3ccc4c5ccccc5n(-c5ccccc5-c5cccc6ccccc56)c4c3)ccc2-c2cccc3ccccc23)cc1]

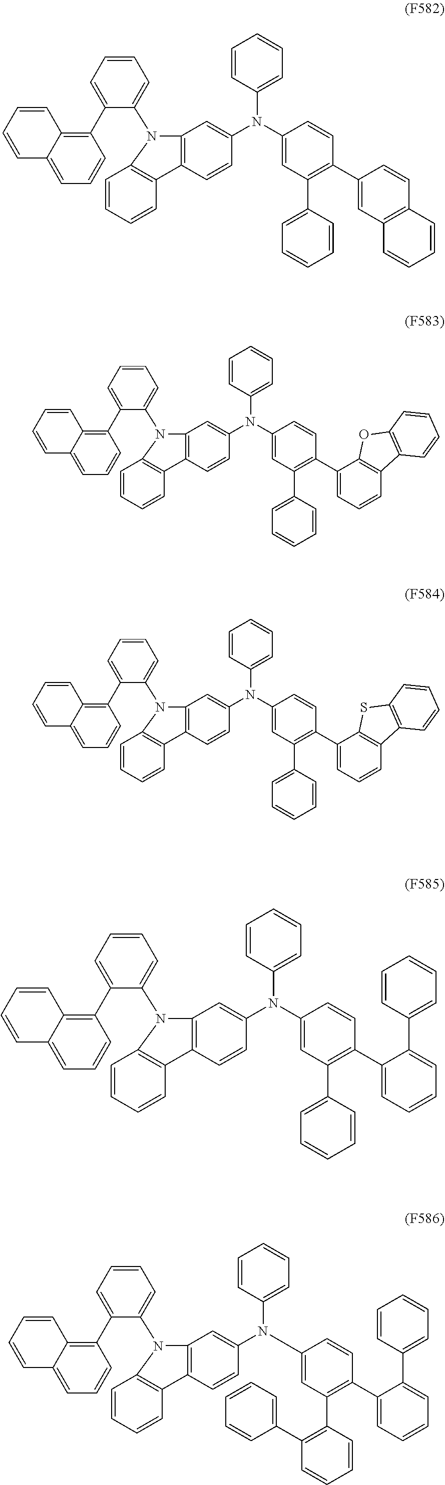 [SMILES: c1ccc(-c2cc(N(c3ccccc3)c3ccc4c5ccccc5n(-c5ccccc5-c5cccc6ccccc56)c4c3)ccc2-c2ccc3ccccc3c2)cc1 ; c1ccc(-c2cc(N(c3ccccc3)c3ccc4c5ccccc5n(-c5ccccc5-c5cccc6ccccc56)c4c3)ccc2-c2cccc3c2oc2ccccc23)cc1 ; c1ccc(-c2cc(N(c3ccccc3)c3ccc4c5ccccc5n(-c5ccccc5-c5cccc6ccccc56)c4c3)ccc2-c2cccc3c2sc2ccccc23)cc1 ; c1ccc(-c2ccccc2-c2ccc(N(c3ccccc3)c3ccc4c5ccccc5n(-c5ccccc5-c5cccc6ccccc56)c4c3)cc2-c2ccccc2)cc1 ; c1ccc(-c2ccccc2-c2ccc(N(c3ccccc3)c3ccc4c5ccccc5n(-c5ccccc5-c5cccc6ccccc56)c4c3)cc2-c2ccccc2-c2ccccc2)cc1]

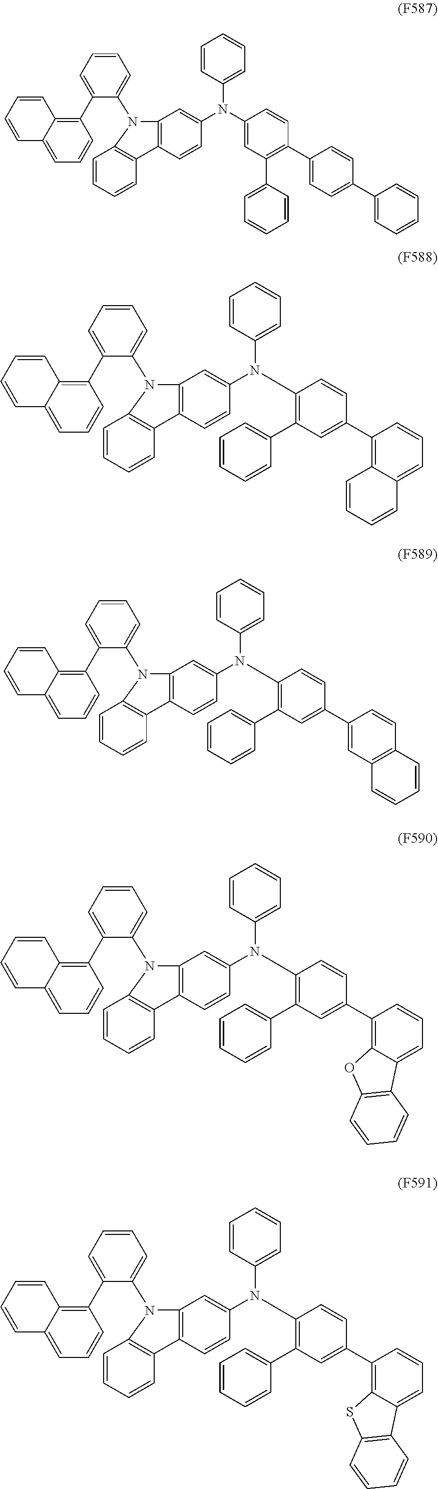 [SMILES: c1ccc(-c2ccc(-c3ccc(N(c4ccccc4)c4ccc5c6ccccc6n(-c6ccccc6-c6cccc7ccccc67)c5c4)cc3-c3ccccc3)cc2)cc1 ; c1ccc(-c2cc(-c3cccc4ccccc34)ccc2N(c2ccccc2)c2ccc3c4ccccc4n(-c4ccccc4-c4cccc5ccccc45)c3c2)cc1 ; c1ccc(-c2cc(-c3ccc4ccccc4c3)ccc2N(c2ccccc2)c2ccc3c4ccccc4n(-c4ccccc4-c4cccc5ccccc45)c3c2)cc1 ; c1ccc(-c2cc(-c3cccc4c3oc3ccccc34)ccc2N(c2ccccc2)c2ccc3c4ccccc4n(-c4ccccc4-c4cccc5ccccc45)c3c2)cc1 ; c1ccc(-c2cc(-c3cccc4c3sc3ccccc34)ccc2N(c2ccccc2)c2ccc3c4ccccc4n(-c4ccccc4-c4cccc5ccccc45)c3c2)cc1]

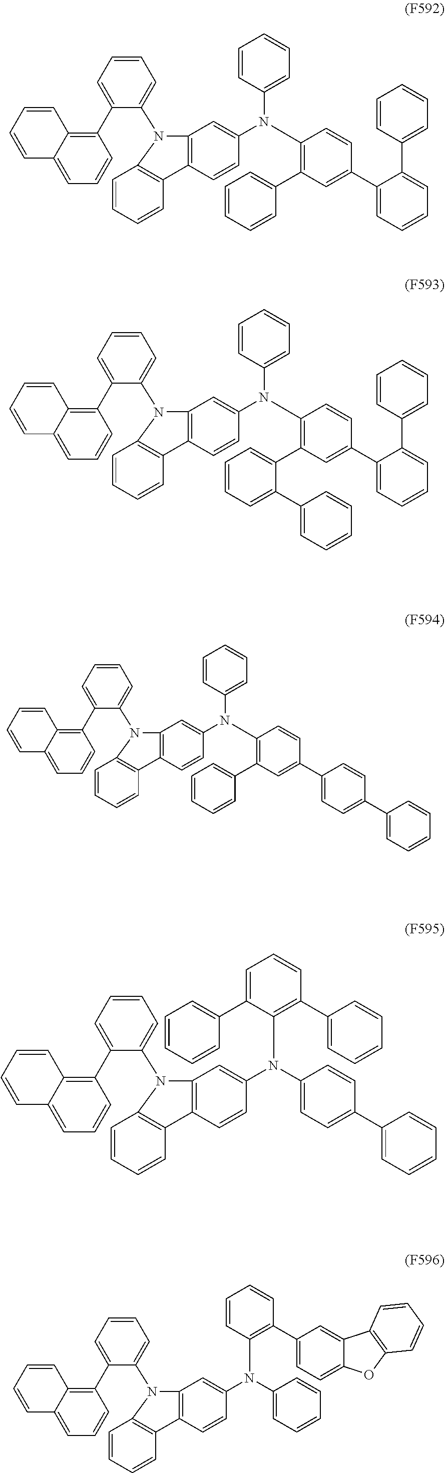 [SMILES: c1ccc(-c2ccccc2-c2ccc(N(c3ccccc3)c3ccc4c5ccccc5n(-c5ccccc5-c5cccc6ccccc56)c4c3)c(-c3ccccc3)c2)cc1 ; c1ccc(-c2ccccc2-c2ccc(N(c3ccccc3)c3ccc4c5ccccc5n(-c5ccccc5-c5cccc6ccccc56)c4c3)c(-c3ccccc3-c3ccccc3)c2)cc1 ; c1ccc(-c2ccc(-c3ccc(N(c4ccccc4)c4ccc5c6ccccc6n(-c6ccccc6-c6cccc7ccccc67)c5c4)c(-c4ccccc4)c3)cc2)cc1 ; c1ccc(-c2ccc(N(c3ccc4c5ccccc5n(-c5ccccc5-c5cccc6ccccc56)c4c3)c3c(-c4ccccc4)cccc3-c3ccccc3)cc2)cc1 ; c1ccc(N(c2ccc3c4ccccc4n(-c4ccccc4-c4cccc5ccccc45)c3c2)c2ccccc2-c2ccc3oc4ccccc4c3c2)cc1]

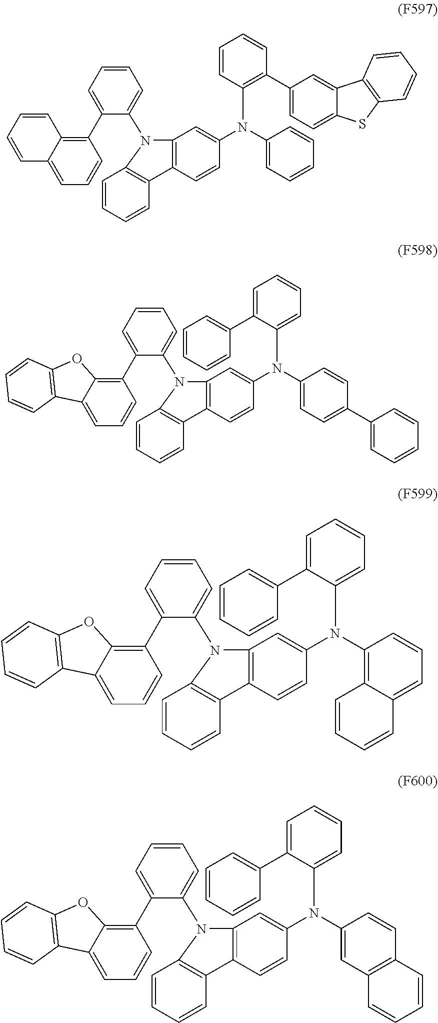 [SMILES: c1ccc(N(c2ccc3c4ccccc4n(-c4ccccc4-c4cccc5ccccc45)c3c2)c2ccccc2-c2ccc3sc4ccccc4c3c2)cc1 ; c1ccc(-c2ccc(N(c3ccc4c5ccccc5n(-c5ccccc5-c5cccc6c5oc5ccccc56)c4c3)c3ccccc3-c3ccccc3)cc2)cc1 ; c1ccc(-c2ccccc2N(c2ccc3c4ccccc4n(-c4ccccc4-c4cccc5c4oc4ccccc45)c3c2)c2cccc3ccccc23)cc1 ; c1ccc(-c2ccccc2N(c2ccc3ccccc3c2)c2ccc3c4ccccc4n(-c4ccccc4-c4cccc5c4oc4ccccc45)c3c2)cc1]

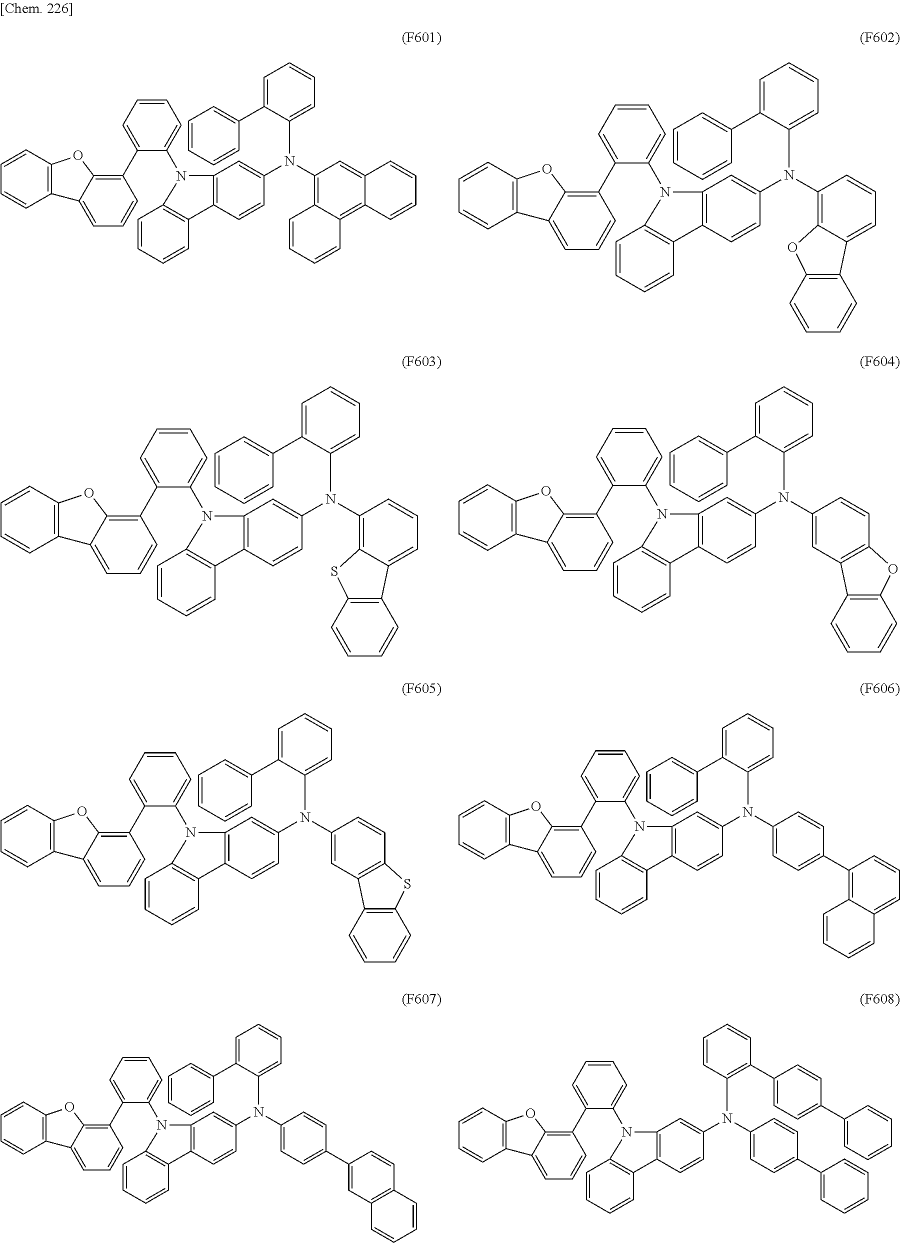

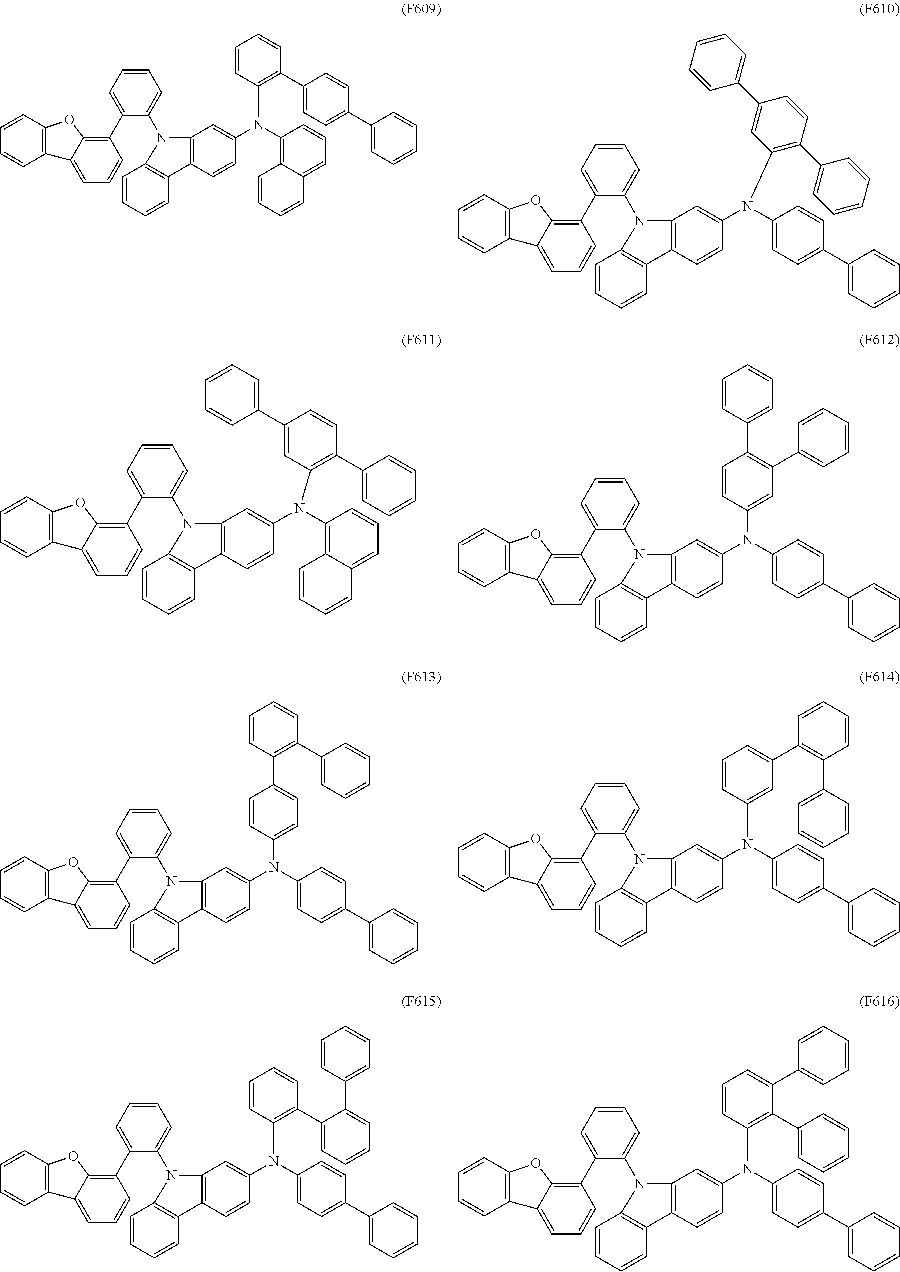

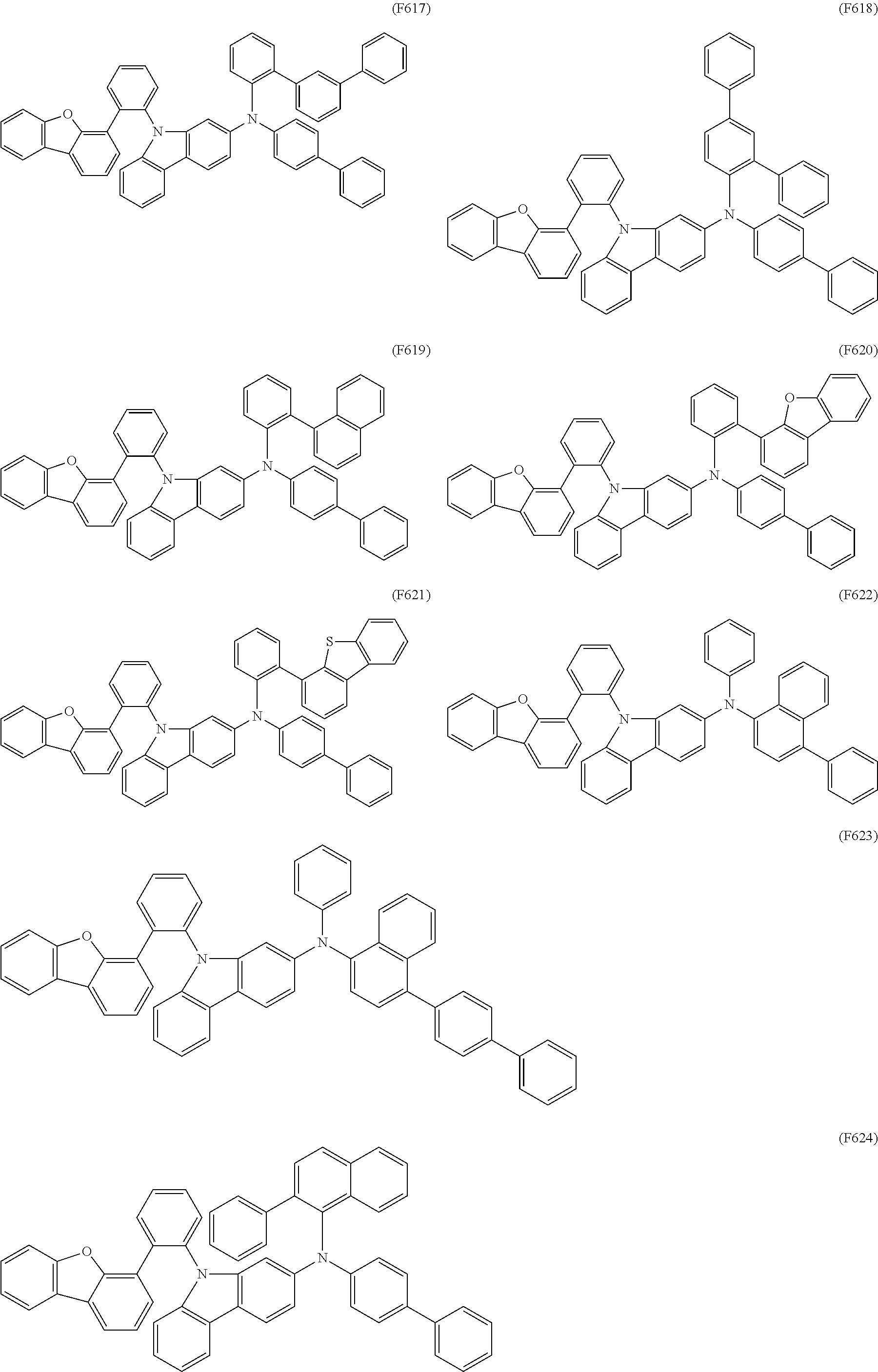

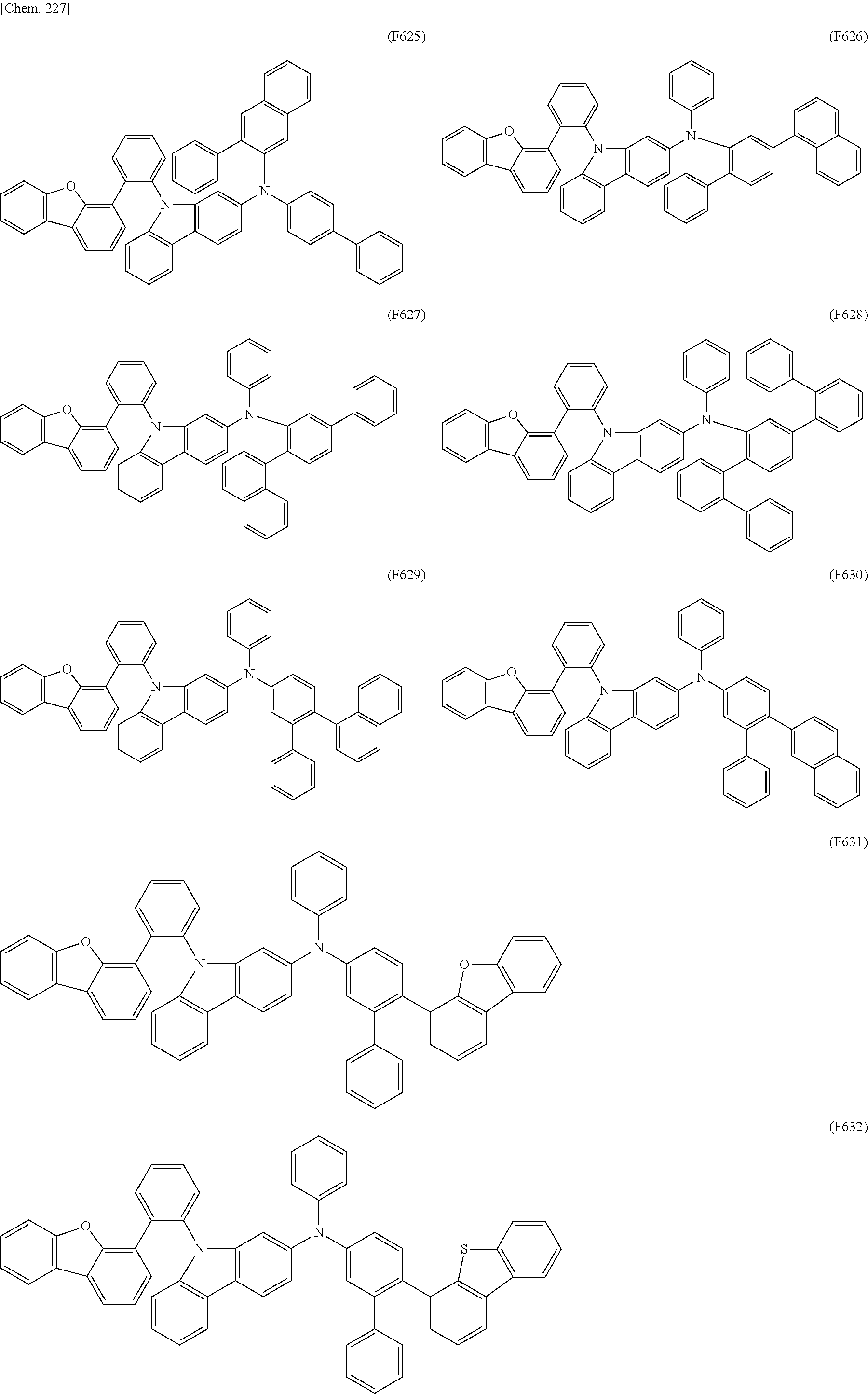

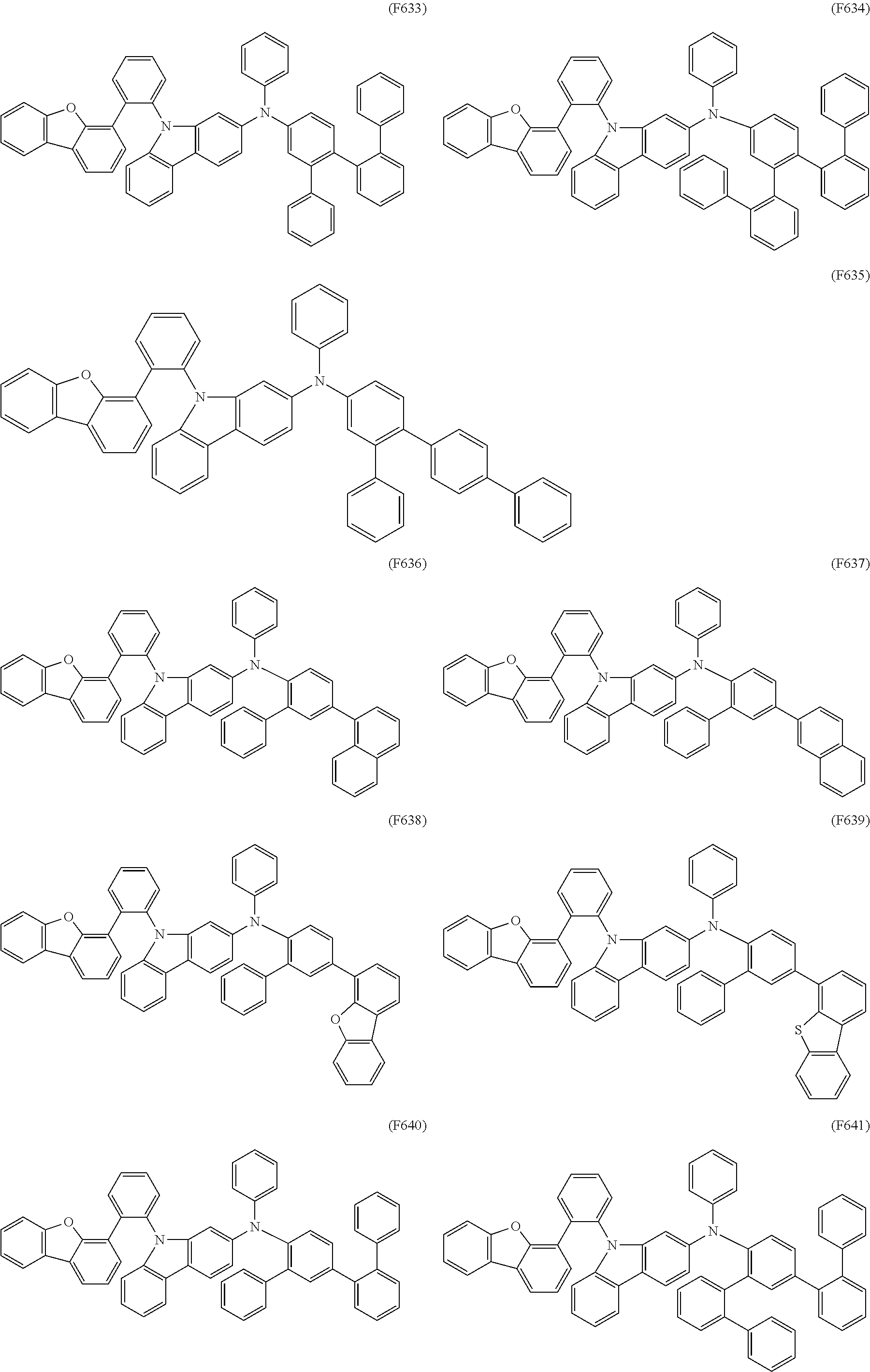

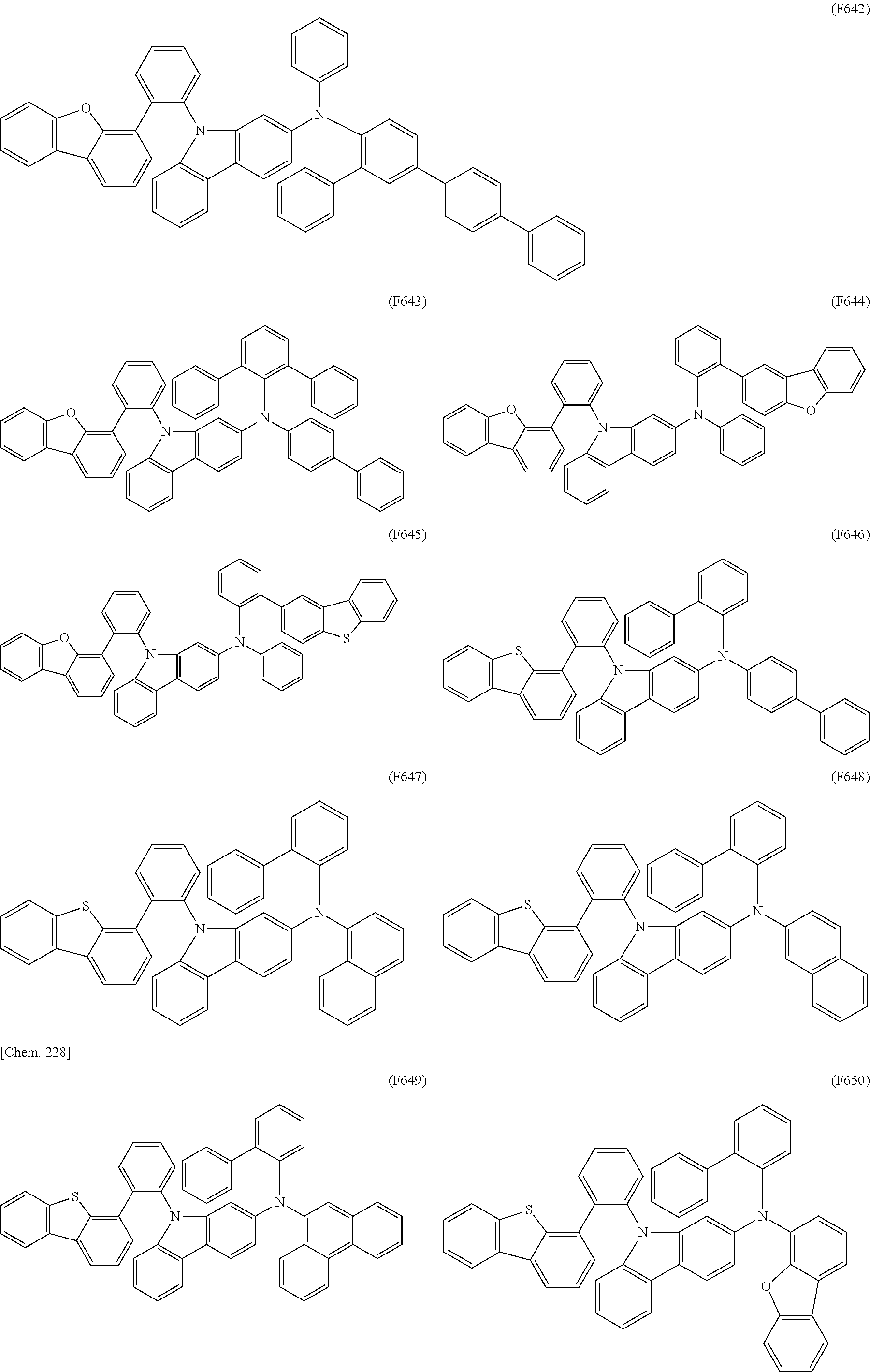

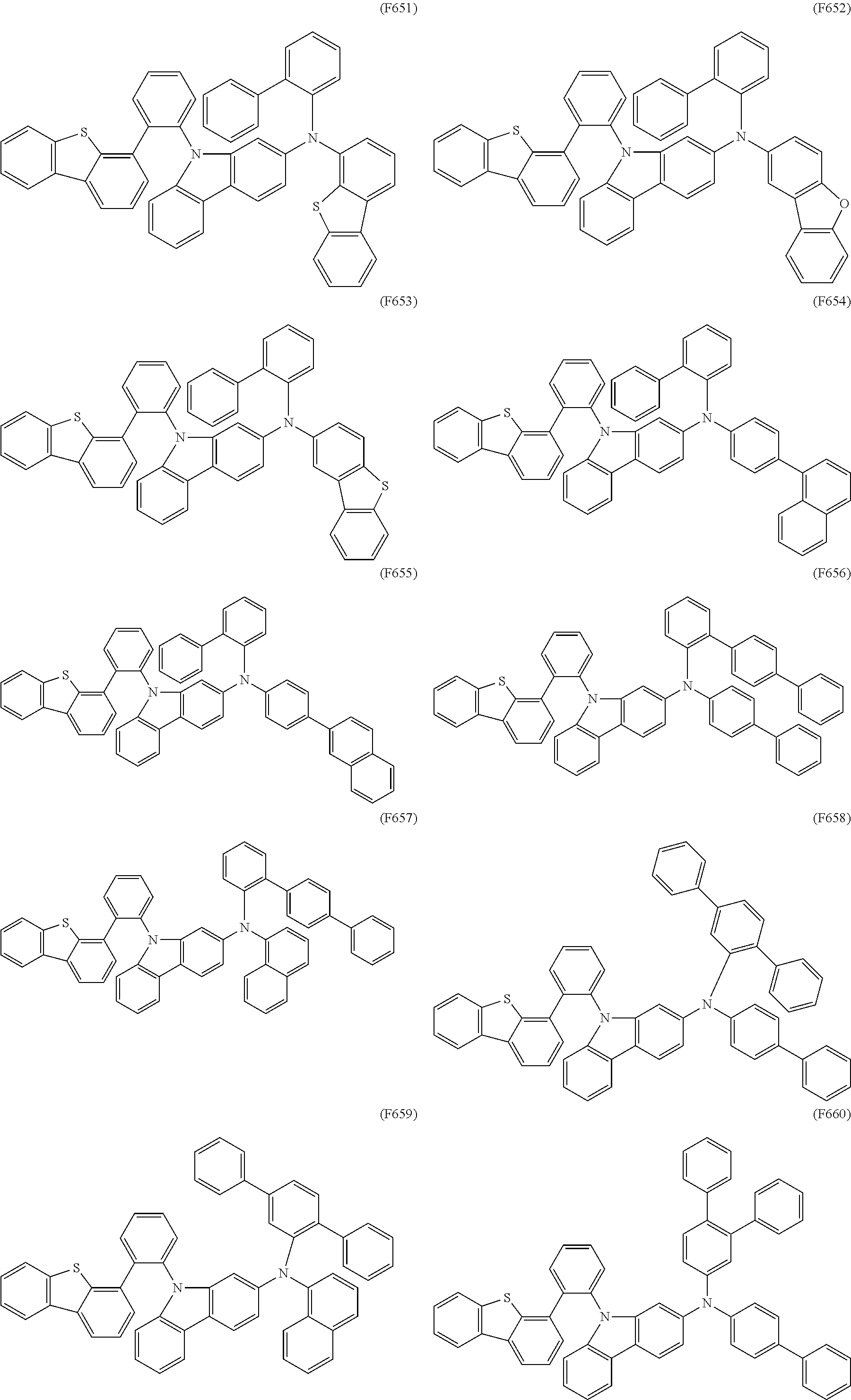

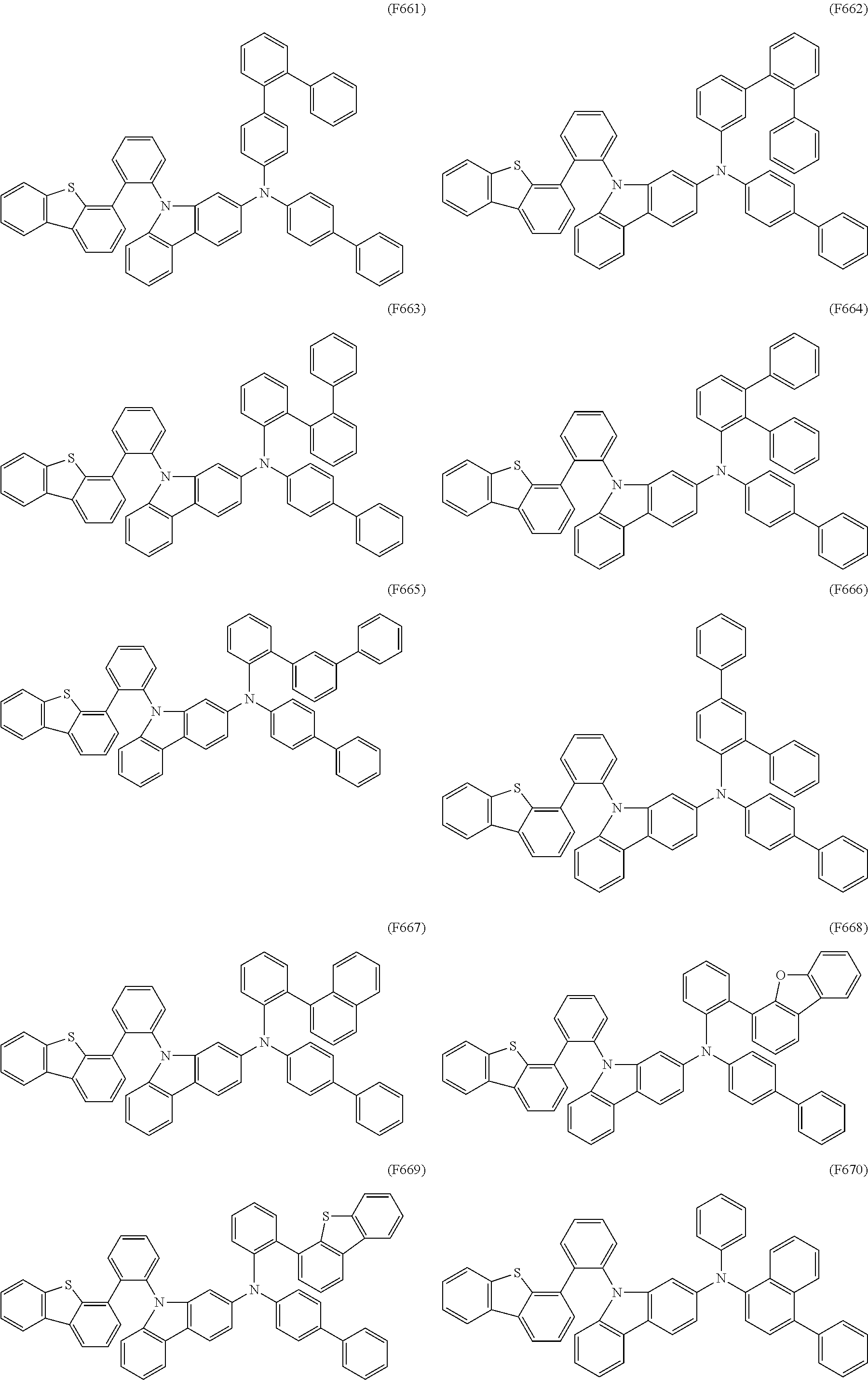

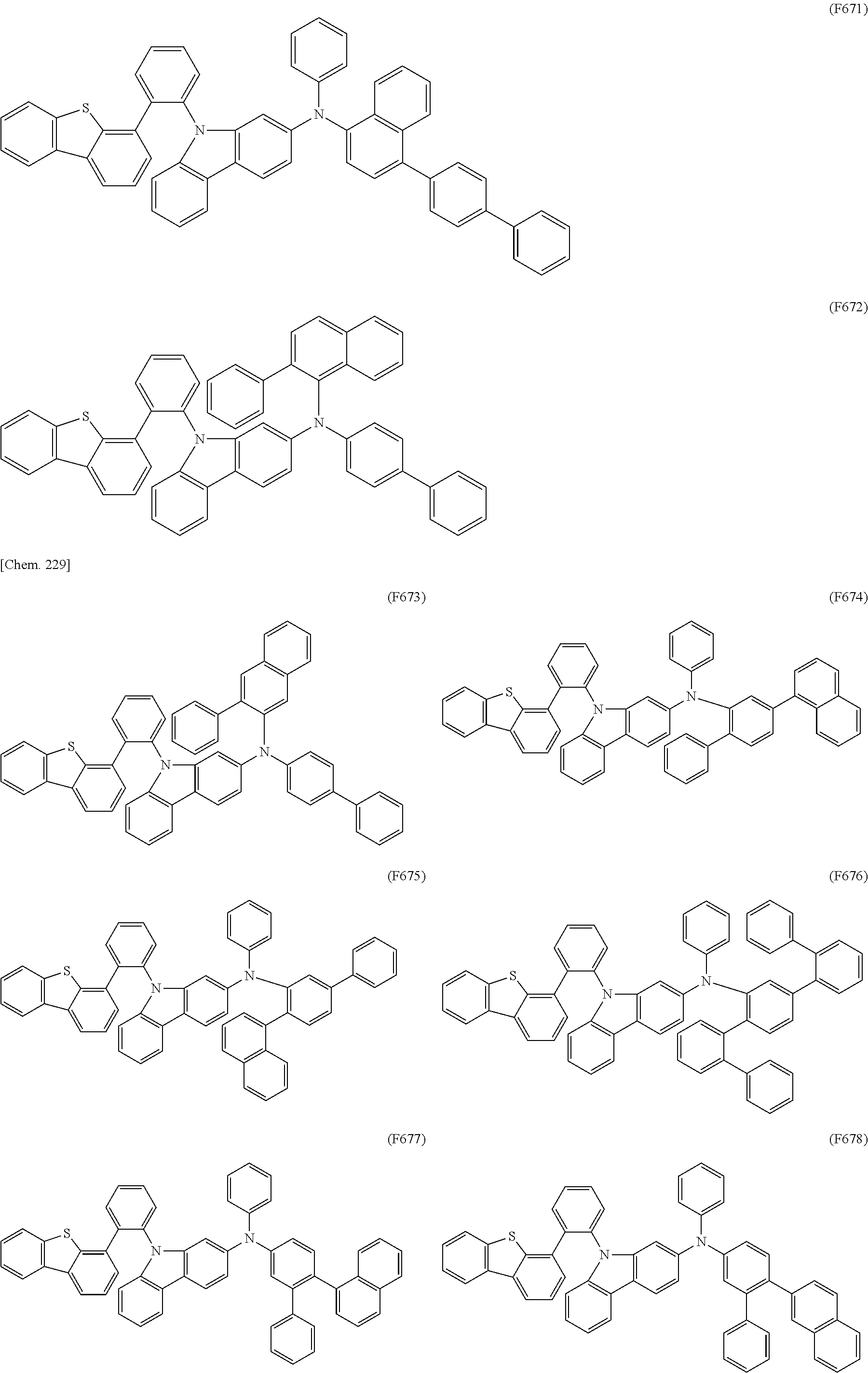

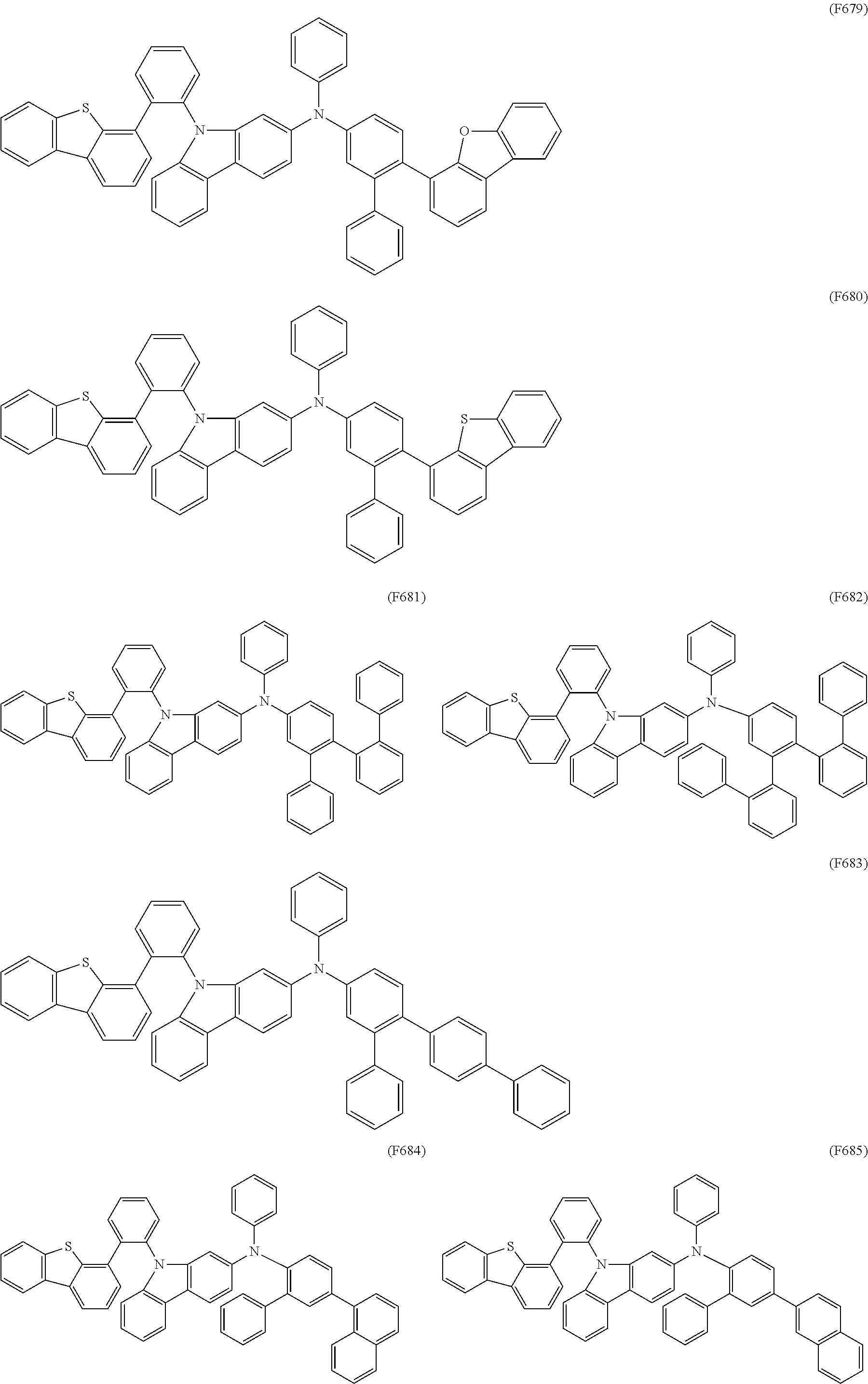

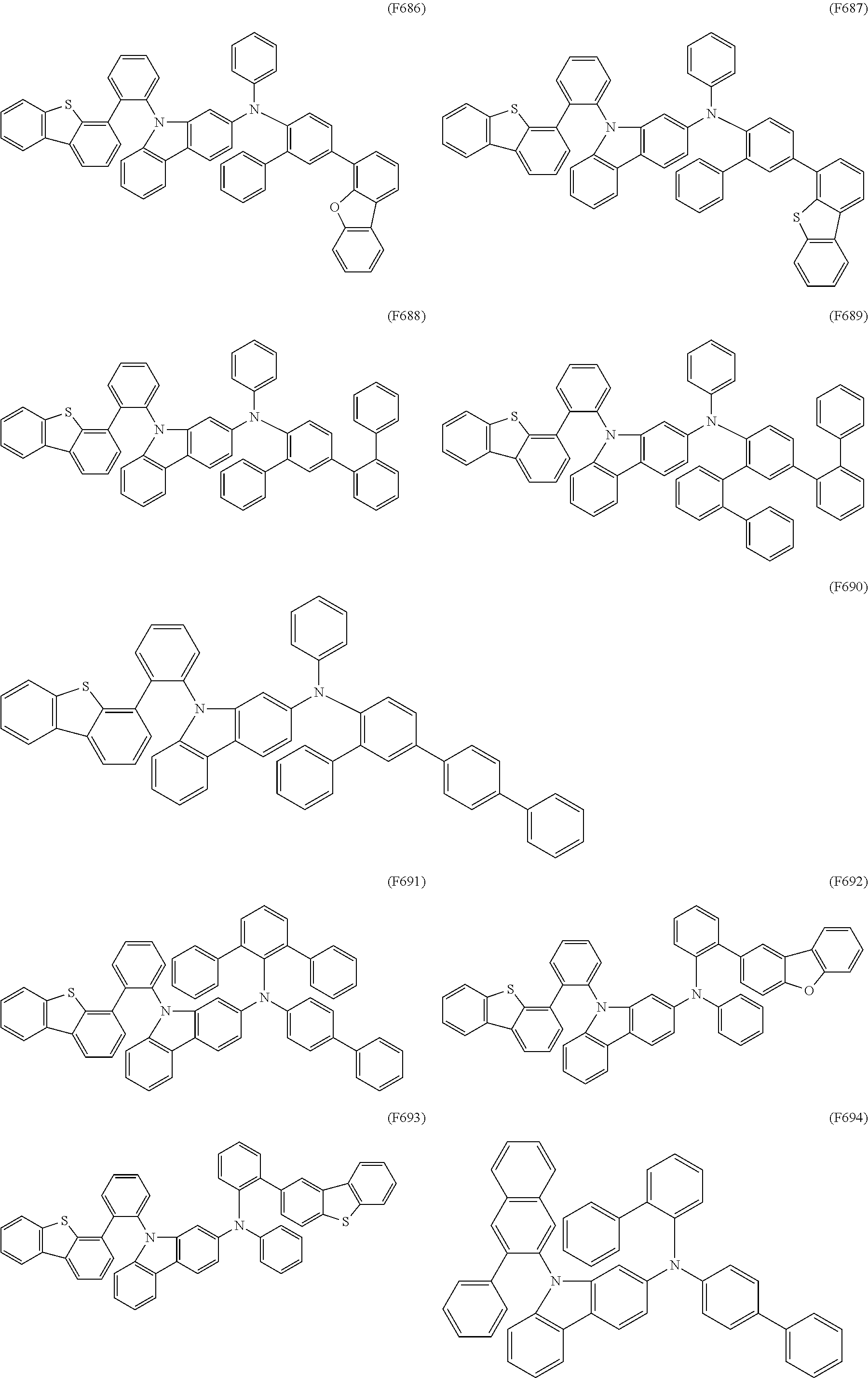

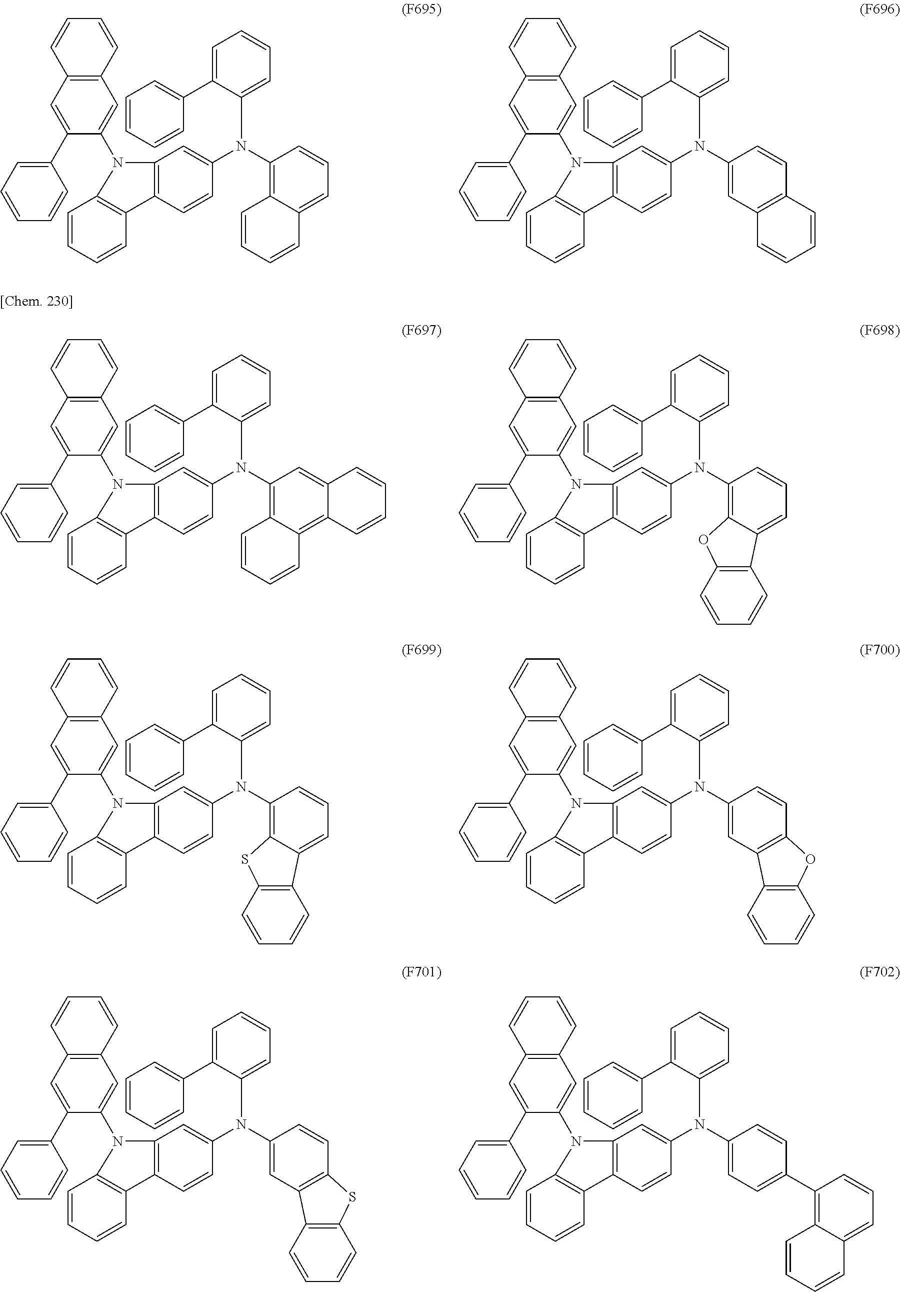

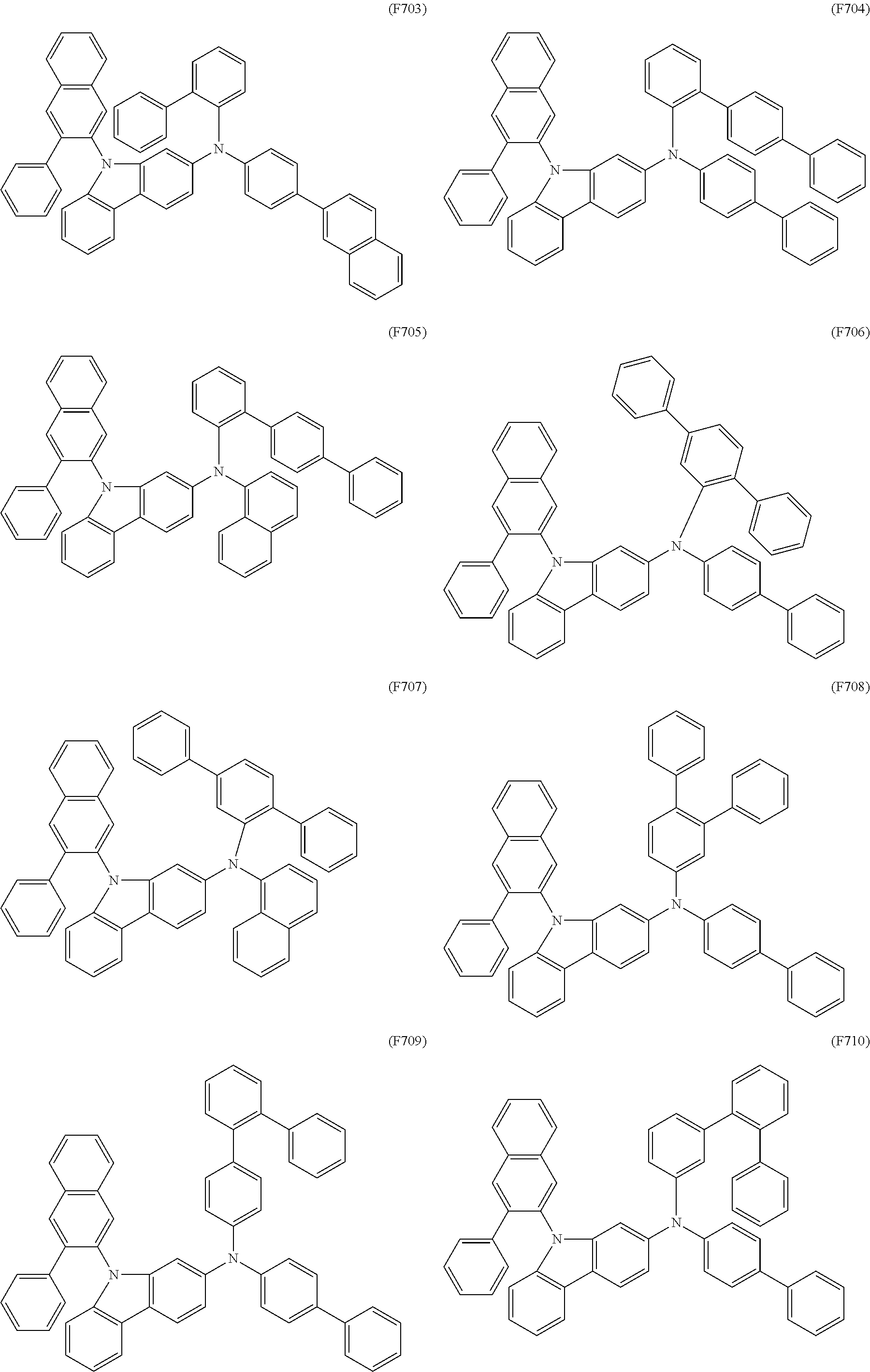

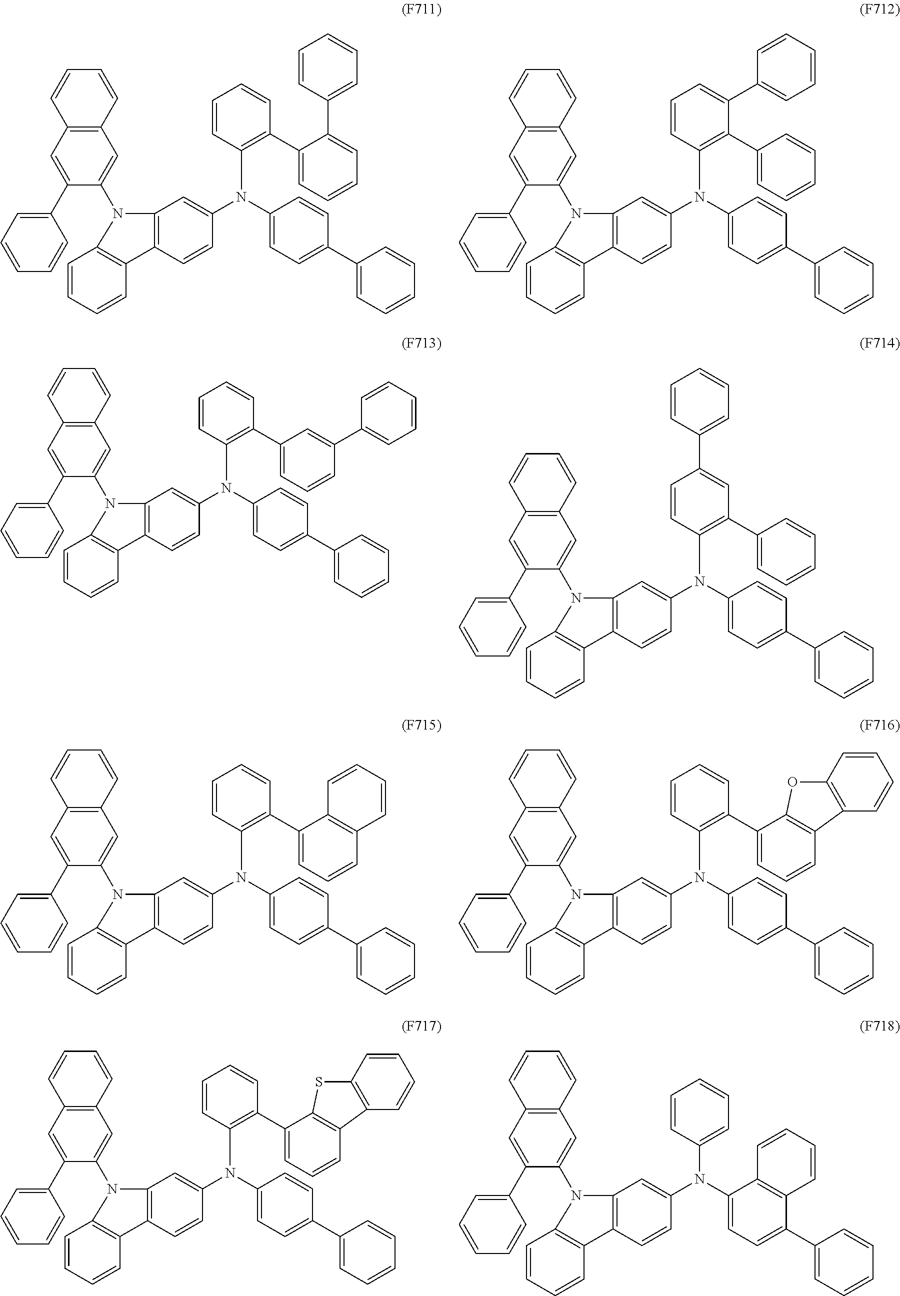

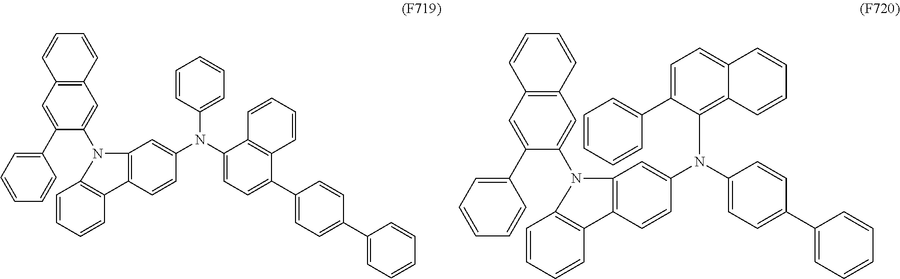 [SMILES: c1ccc(-c2ccc(-c3ccc(N(c4ccccc4)c4ccc5c6ccccc6n(-c6cc7ccccc7cc6-c6ccccc6)c5c4)c4ccccc34)cc2)cc1 ; c1ccc(-c2ccc(N(c3ccc4c5ccccc5n(-c5cc6ccccc6cc5-c5ccccc5)c4c3)c3c(-c4ccccc4)ccc4ccccc34)cc2)cc1]

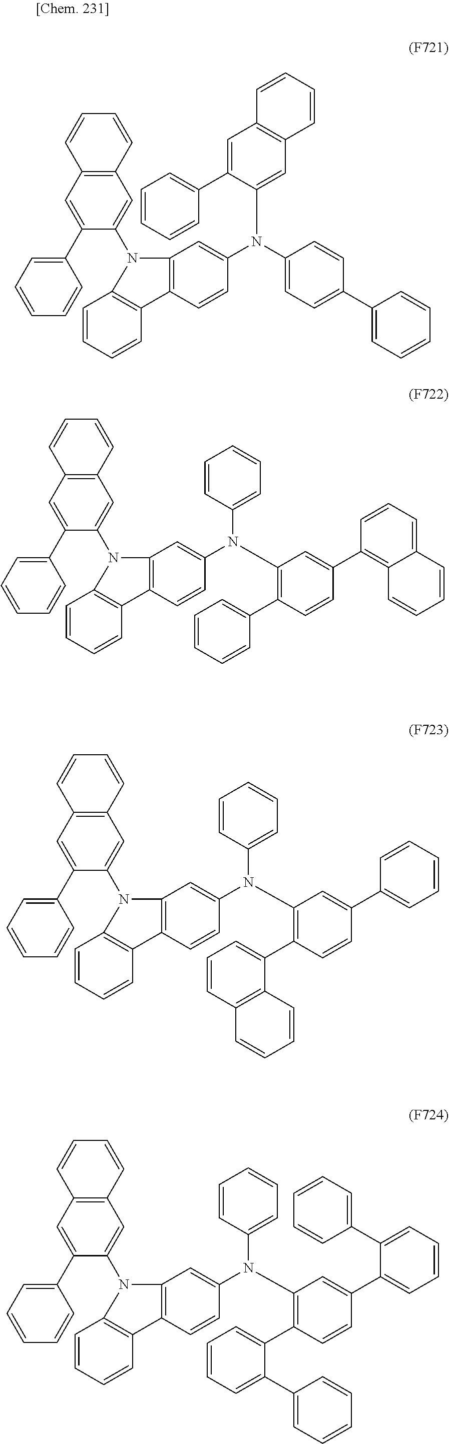 [SMILES: c1ccc(-c2ccc(N(c3ccc4c5ccccc5n(-c5cc6ccccc6cc5-c5ccccc5)c4c3)c3cc4ccccc4cc3-c3ccccc3)cc2)cc1 ; c1ccc(-c2ccc(-c3cccc4ccccc34)cc2N(c2ccccc2)c2ccc3c4ccccc4n(-c4cc5ccccc5cc4-c4ccccc4)c3c2)cc1 ; c1ccc(-c2ccc(-c3cccc4ccccc34)c(N(c3ccccc3)c3ccc4c5ccccc5n(-c5cc6ccccc6cc5-c5ccccc5)c4c3)c2)cc1 ; c1ccc(-c2ccccc2-c2ccc(-c3ccccc3-c3ccccc3)c(N(c3ccccc3)c3ccc4c5ccccc5n(-c5cc6ccccc6cc5-c5ccccc5)c4c3)c2)cc1]

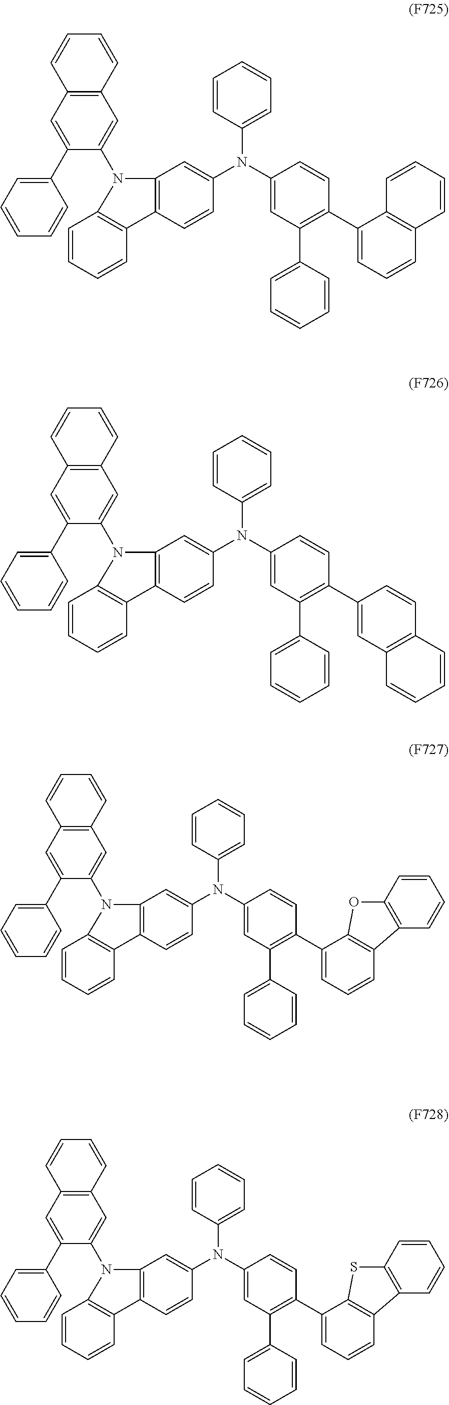 [SMILES: c1ccc(-c2cc(N(c3ccccc3)c3ccc4c5ccccc5n(-c5cc6ccccc6cc5-c5ccccc5)c4c3)ccc2-c2cccc3ccccc23)cc1 ; c1ccc(-c2cc(N(c3ccccc3)c3ccc4c5ccccc5n(-c5cc6ccccc6cc5-c5ccccc5)c4c3)ccc2-c2ccc3ccccc3c2)cc1 ; c1ccc(-c2cc(N(c3ccccc3)c3ccc4c5ccccc5n(-c5cc6ccccc6cc5-c5ccccc5)c4c3)ccc2-c2cccc3c2oc2ccccc23)cc1 ; c1ccc(-c2cc(N(c3ccccc3)c3ccc4c5ccccc5n(-c5cc6ccccc6cc5-c5ccccc5)c4c3)ccc2-c2cccc3c2sc2ccccc23)cc1]

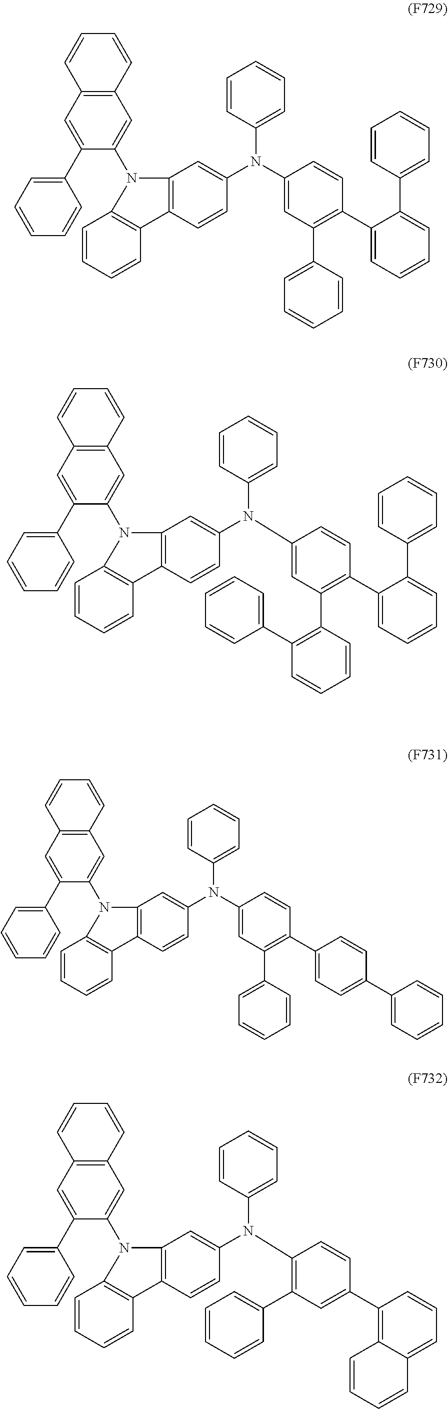 [SMILES: c1ccc(-c2ccccc2-c2ccc(N(c3ccccc3)c3ccc4c5ccccc5n(-c5cc6ccccc6cc5-c5ccccc5)c4c3)cc2-c2ccccc2)cc1 ; c1ccc(-c2ccccc2-c2ccc(N(c3ccccc3)c3ccc4c5ccccc5n(-c5cc6ccccc6cc5-c5ccccc5)c4c3)cc2-c2ccccc2-c2ccccc2)cc1 ; c1ccc(-c2ccc(-c3ccc(N(c4ccccc4)c4ccc5c6ccccc6n(-c6cc7ccccc7cc6-c6ccccc6)c5c4)cc3-c3ccccc3)cc2)cc1 ; c1ccc(-c2cc(-c3cccc4ccccc34)ccc2N(c2ccccc2)c2ccc3c4ccccc4n(-c4cc5ccccc5cc4-c4ccccc4)c3c2)cc1]

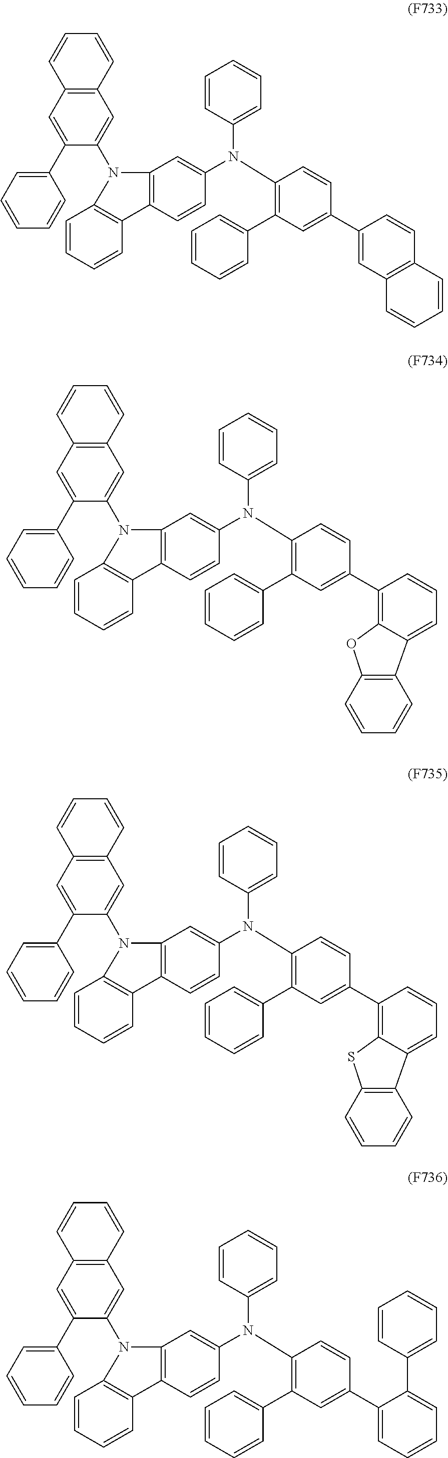 [SMILES: c1ccc(-c2cc(-c3ccc4ccccc4c3)ccc2N(c2ccccc2)c2ccc3c4ccccc4n(-c4cc5ccccc5cc4-c4ccccc4)c3c2)cc1 ; c1ccc(-c2cc(-c3cccc4c3oc3ccccc34)ccc2N(c2ccccc2)c2ccc3c4ccccc4n(-c4cc5ccccc5cc4-c4ccccc4)c3c2)cc1 ; c1ccc(-c2cc(-c3cccc4c3sc3ccccc34)ccc2N(c2ccccc2)c2ccc3c4ccccc4n(-c4cc5ccccc5cc4-c4ccccc4)c3c2)cc1 ; c1ccc(-c2ccccc2-c2ccc(N(c3ccccc3)c3ccc4c5ccccc5n(-c5cc6ccccc6cc5-c5ccccc5)c4c3)c(-c3ccccc3)c2)cc1]

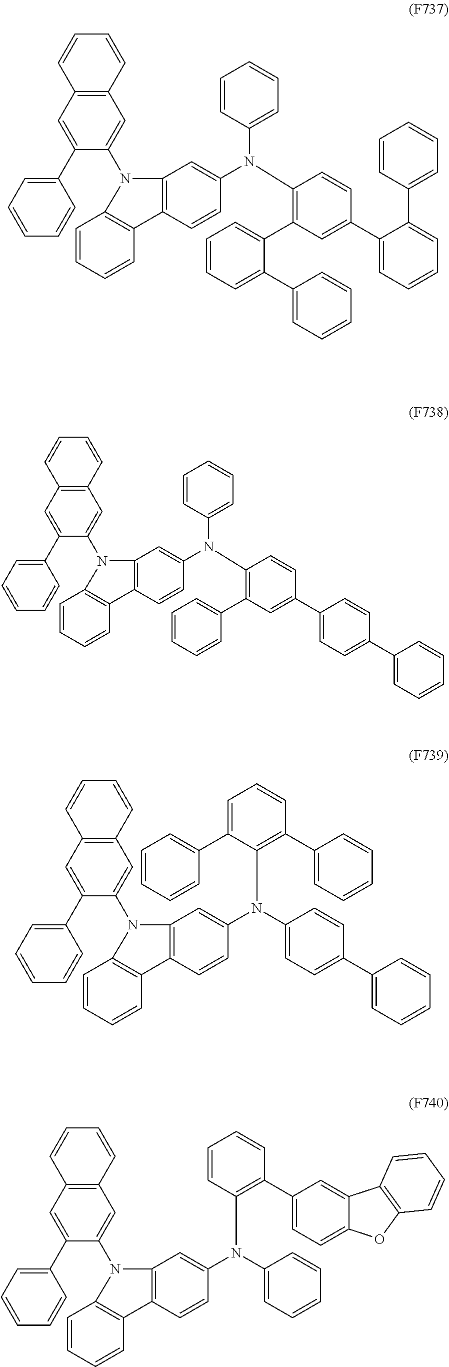 [SMILES: c1ccc(-c2ccccc2-c2ccc(N(c3ccccc3)c3ccc4c5ccccc5n(-c5cc6ccccc6cc5-c5ccccc5)c4c3)c(-c3ccccc3-c3ccccc3)c2)cc1 ; c1ccc(-c2ccc(-c3ccc(N(c4ccccc4)c4ccc5c6ccccc6n(-c6cc7ccccc7cc6-c6ccccc6)c5c4)c(-c4ccccc4)c3)cc2)cc1 ; c1ccc(-c2ccc(N(c3ccc4c5ccccc5n(-c5cc6ccccc6cc5-c5ccccc5)c4c3)c3c(-c4ccccc4)cccc3-c3ccccc3)cc2)cc1 ; c1ccc(-c2cc3ccccc3cc2-n2c3ccccc3c3ccc(N(c4ccccc4)c4ccccc4-c4ccc5oc6ccccc6c5c4)cc32)cc1]

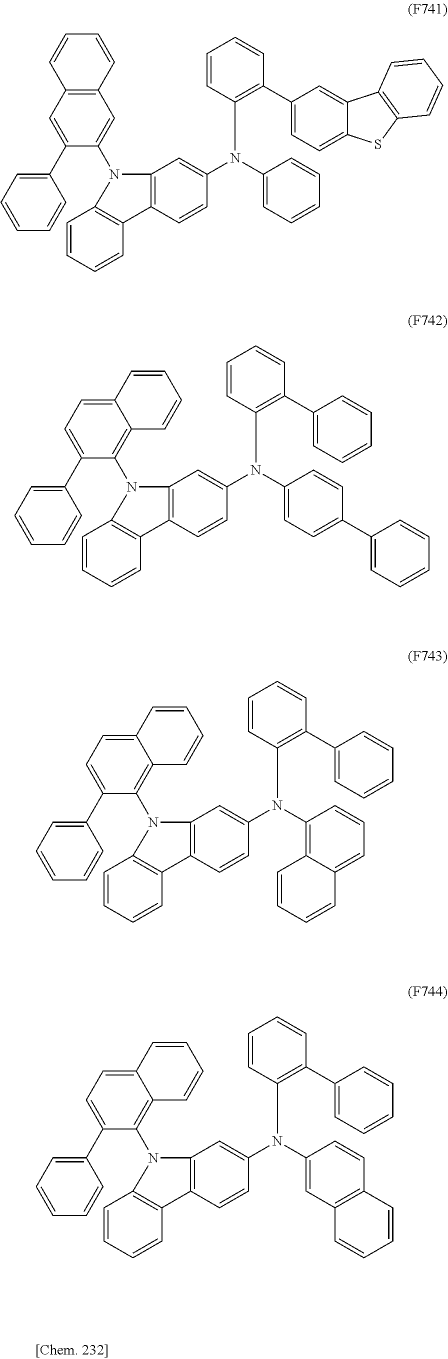 [SMILES: c1ccc(-c2cc3ccccc3cc2-n2c3ccccc3c3ccc(N(c4ccccc4)c4ccccc4-c4ccc5sc6ccccc6c5c4)cc32)cc1 ; c1ccc(-c2ccc(N(c3ccc4c5ccccc5n(-c5c(-c6ccccc6)ccc6ccccc56)c4c3)c3ccccc3-c3ccccc3)cc2)cc1 ; c1ccc(-c2ccccc2N(c2ccc3c4ccccc4n(-c4c(-c5ccccc5)ccc5ccccc45)c3c2)c2cccc3ccccc23)cc1 ; c1ccc(-c2ccccc2N(c2ccc3ccccc3c2)c2ccc3c4ccccc4n(-c4c(-c5ccccc5)ccc5ccccc45)c3c2)cc1]

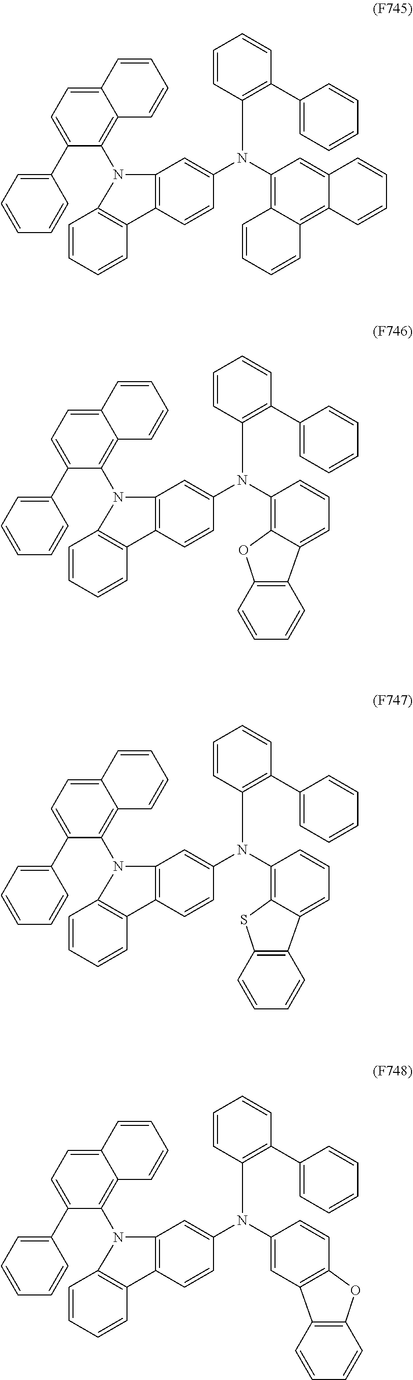 [SMILES: c1ccc(-c2ccccc2N(c2ccc3c4ccccc4n(-c4c(-c5ccccc5)ccc5ccccc45)c3c2)c2cc3ccccc3c3ccccc23)cc1 ; c1ccc(-c2ccccc2N(c2ccc3c4ccccc4n(-c4c(-c5ccccc5)ccc5ccccc45)c3c2)c2cccc3c2oc2ccccc23)cc1 ; c1ccc(-c2ccccc2N(c2ccc3c4ccccc4n(-c4c(-c5ccccc5)ccc5ccccc45)c3c2)c2cccc3c2sc2ccccc23)cc1 ; c1ccc(-c2ccccc2N(c2ccc3oc4ccccc4c3c2)c2ccc3c4ccccc4n(-c4c(-c5ccccc5)ccc5ccccc45)c3c2)cc1]

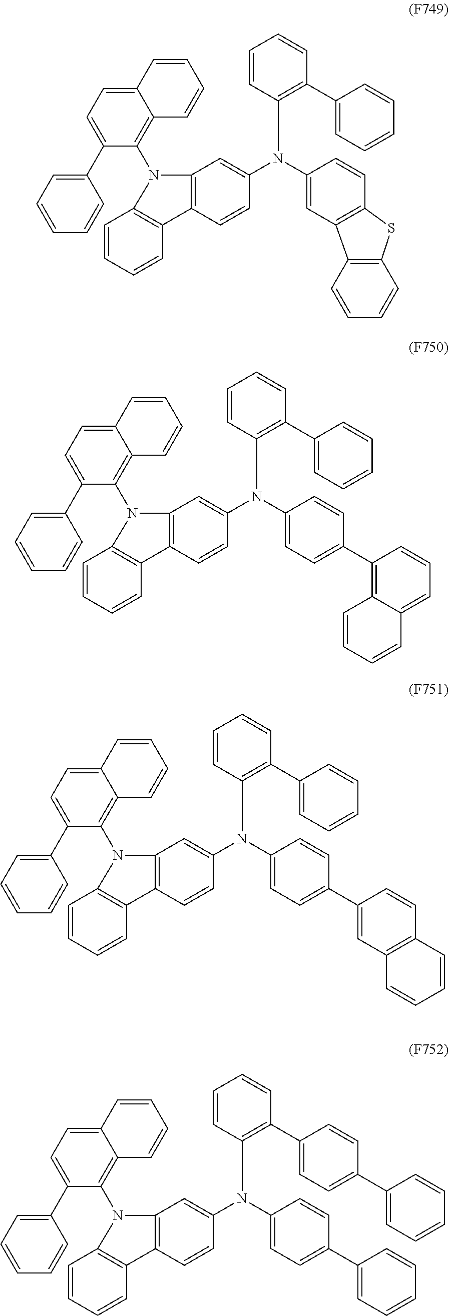 [SMILES: c1ccc(-c2ccccc2N(c2ccc3sc4ccccc4c3c2)c2ccc3c4ccccc4n(-c4c(-c5ccccc5)ccc5ccccc45)c3c2)cc1 ; c1ccc(-c2ccccc2N(c2ccc(-c3cccc4ccccc34)cc2)c2ccc3c4ccccc4n(-c4c(-c5ccccc5)ccc5ccccc45)c3c2)cc1 ; c1ccc(-c2ccccc2N(c2ccc(-c3ccc4ccccc4c3)cc2)c2ccc3c4ccccc4n(-c4c(-c5ccccc5)ccc5ccccc45)c3c2)cc1 ; c1ccc(-c2ccc(-c3ccccc3N(c3ccc(-c4ccccc4)cc3)c3ccc4c5ccccc5n(-c5c(-c6ccccc6)ccc6ccccc56)c4c3)cc2)cc1]

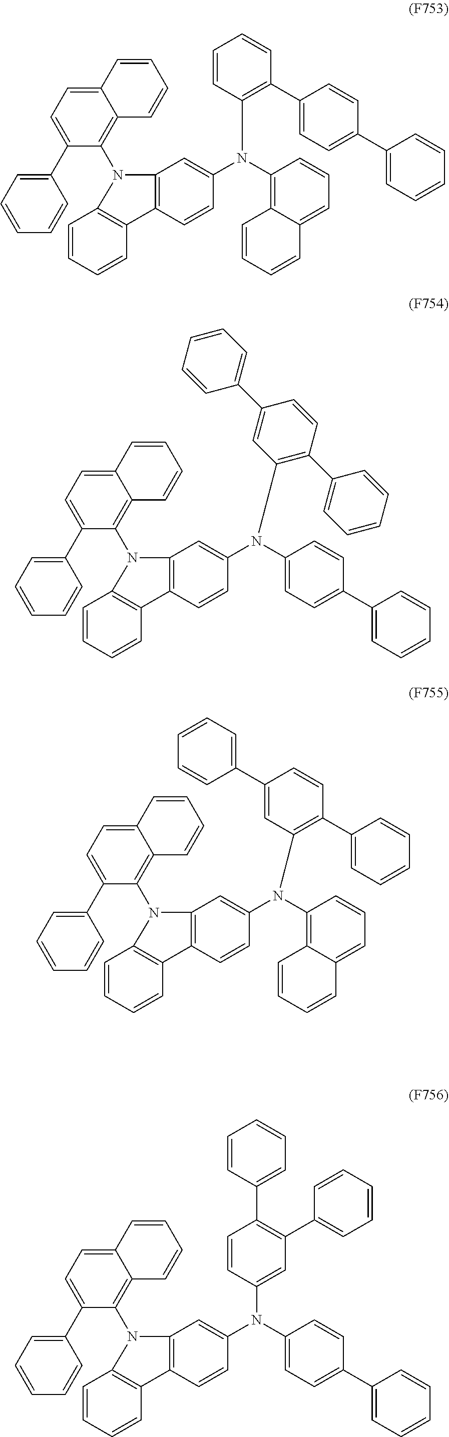 [SMILES: c1ccc(-c2ccc(-c3ccccc3N(c3ccc4c5ccccc5n(-c5c(-c6ccccc6)ccc6ccccc56)c4c3)c3cccc4ccccc34)cc2)cc1 ; c1ccc(-c2ccc(N(c3ccc4c5ccccc5n(-c5c(-c6ccccc6)ccc6ccccc56)c4c3)c3cc(-c4ccccc4)ccc3-c3ccccc3)cc2)cc1 ; c1ccc(-c2ccc(-c3ccccc3)c(N(c3ccc4c5ccccc5n(-c5c(-c6ccccc6)ccc6ccccc56)c4c3)c3cccc4ccccc34)c2)cc1 ; c1ccc(-c2ccc(N(c3ccc(-c4ccccc4)c(-c4ccccc4)c3)c3ccc4c5ccccc5n(-c5c(-c6ccccc6)ccc6ccccc56)c4c3)cc2)cc1]

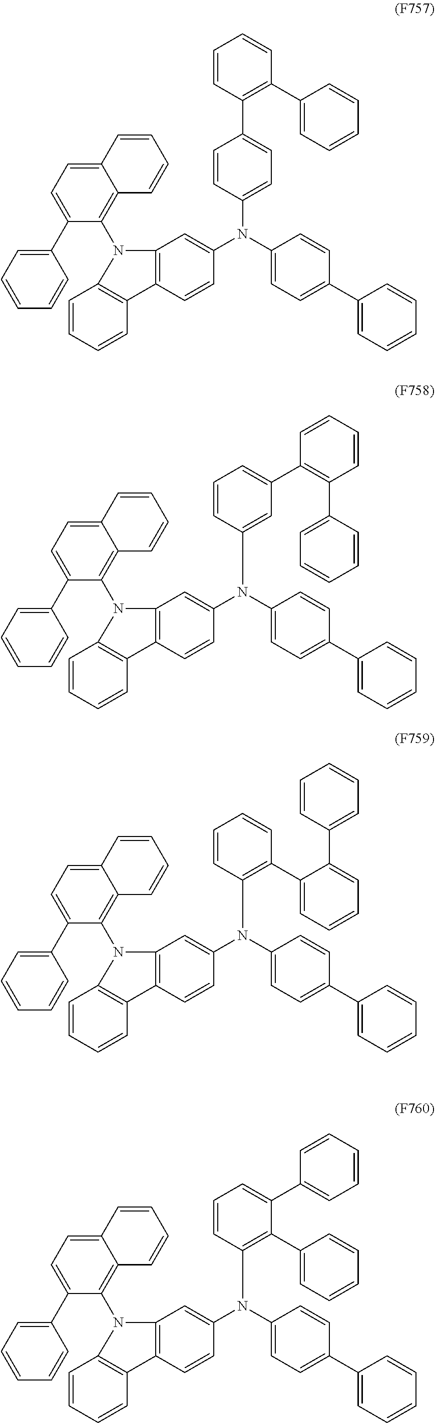 [SMILES: c1ccc(-c2ccc(N(c3ccc(-c4ccccc4-c4ccccc4)cc3)c3ccc4c5ccccc5n(-c5c(-c6ccccc6)ccc6ccccc56)c4c3)cc2)cc1 ; c1ccc(-c2ccc(N(c3cccc(-c4ccccc4-c4ccccc4)c3)c3ccc4c5ccccc5n(-c5c(-c6ccccc6)ccc6ccccc56)c4c3)cc2)cc1 ; c1ccc(-c2ccc(N(c3ccc4c5ccccc5n(-c5c(-c6ccccc6)ccc6ccccc56)c4c3)c3ccccc3-c3ccccc3-c3ccccc3)cc2)cc1 ; c1ccc(-c2ccc(N(c3ccc4c5ccccc5n(-c5c(-c6ccccc6)ccc6ccccc56)c4c3)c3cccc(-c4ccccc4)c3-c3ccccc3)cc2)cc1]

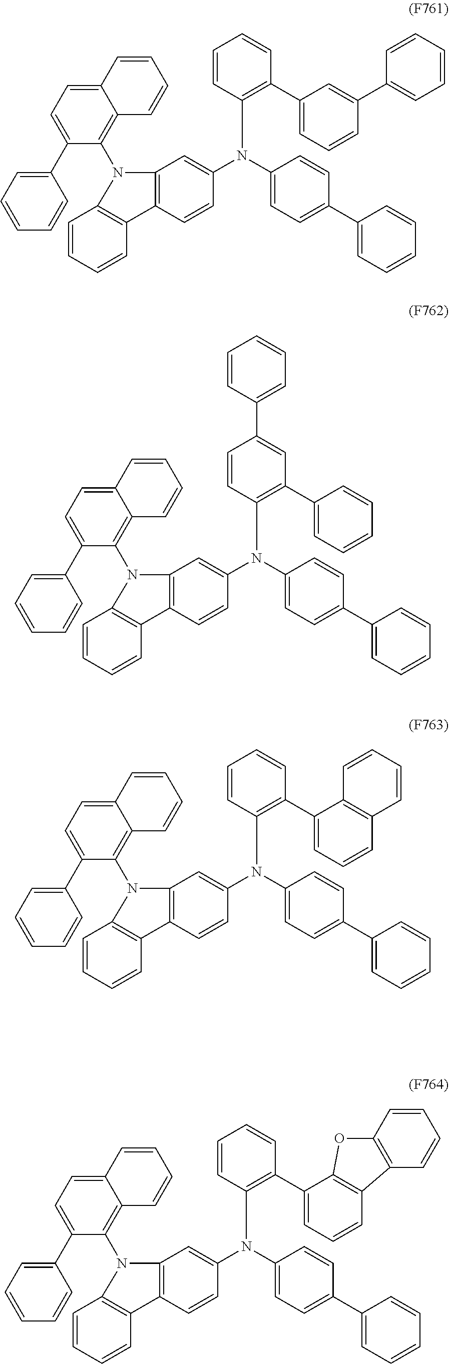 [SMILES: c1ccc(-c2ccc(N(c3ccc4c5ccccc5n(-c5c(-c6ccccc6)ccc6ccccc56)c4c3)c3ccccc3-c3cccc(-c4ccccc4)c3)cc2)cc1 ; c1ccc(-c2ccc(N(c3ccc4c5ccccc5n(-c5c(-c6ccccc6)ccc6ccccc56)c4c3)c3ccc(-c4ccccc4)cc3-c3ccccc3)cc2)cc1 ; c1ccc(-c2ccc(N(c3ccc4c5ccccc5n(-c5c(-c6ccccc6)ccc6ccccc56)c4c3)c3ccccc3-c3cccc4ccccc34)cc2)cc1 ; c1ccc(-c2ccc(N(c3ccc4c5ccccc5n(-c5c(-c6ccccc6)ccc6ccccc56)c4c3)c3ccccc3-c3cccc4c3oc3ccccc34)cc2)cc1]

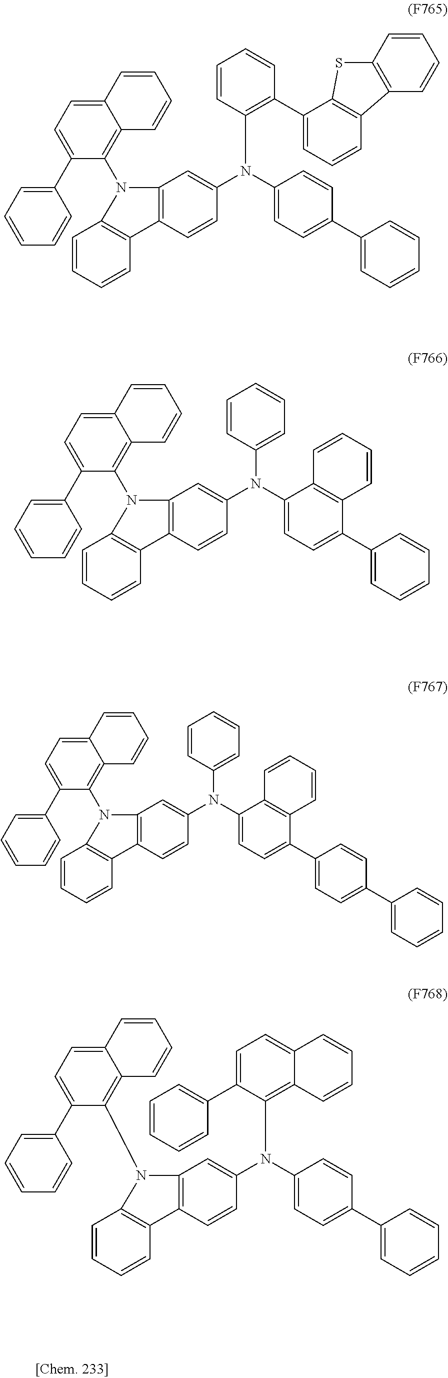 [SMILES: c1ccc(-c2ccc(N(c3ccc4c5ccccc5n(-c5c(-c6ccccc6)ccc6ccccc56)c4c3)c3ccccc3-c3cccc4c3sc3ccccc34)cc2)cc1 ; c1ccc(-c2ccc3ccccc3c2-n2c3ccccc3c3ccc(N(c4ccccc4)c4ccc(-c5ccccc5)c5ccccc45)cc32)cc1 ; c1ccc(-c2ccc(-c3ccc(N(c4ccccc4)c4ccc5c6ccccc6n(-c6c(-c7ccccc7)ccc7ccccc67)c5c4)c4ccccc34)cc2)cc1 ; c1ccc(-c2ccc(N(c3ccc4c5ccccc5n(-c5c(-c6ccccc6)ccc6ccccc56)c4c3)c3c(-c4ccccc4)ccc4ccccc34)cc2)cc1]

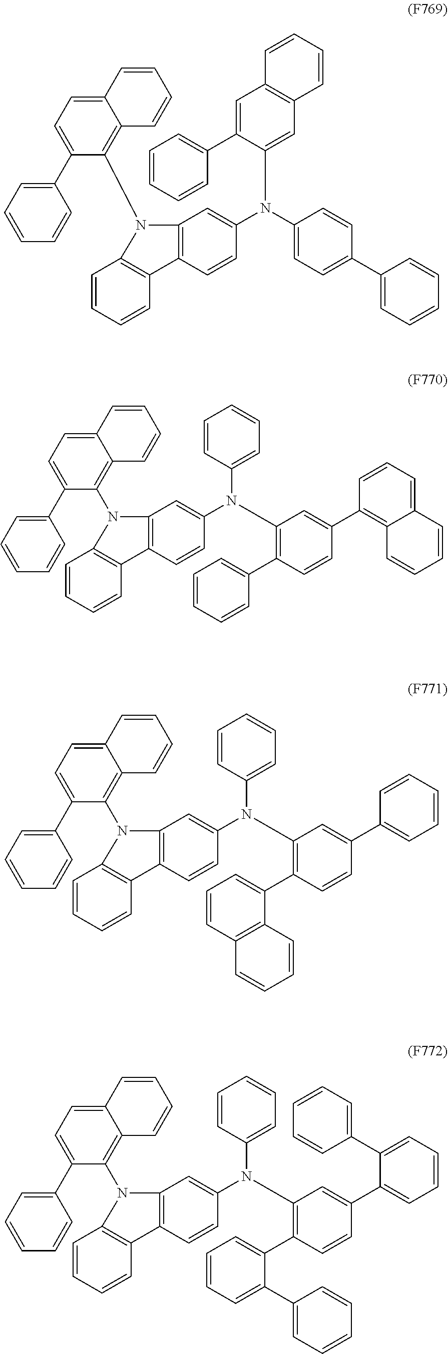 [SMILES: c1ccc(-c2ccc(N(c3ccc4c5ccccc5n(-c5c(-c6ccccc6)ccc6ccccc56)c4c3)c3cc4ccccc4cc3-c3ccccc3)cc2)cc1 ; c1ccc(-c2ccc(-c3cccc4ccccc34)cc2N(c2ccccc2)c2ccc3c4ccccc4n(-c4c(-c5ccccc5)ccc5ccccc45)c3c2)cc1 ; c1ccc(-c2ccc(-c3cccc4ccccc34)c(N(c3ccccc3)c3ccc4c5ccccc5n(-c5c(-c6ccccc6)ccc6ccccc56)c4c3)c2)cc1 ; c1ccc(-c2ccccc2-c2ccc(-c3ccccc3-c3ccccc3)c(N(c3ccccc3)c3ccc4c5ccccc5n(-c5c(-c6ccccc6)ccc6ccccc56)c4c3)c2)cc1]

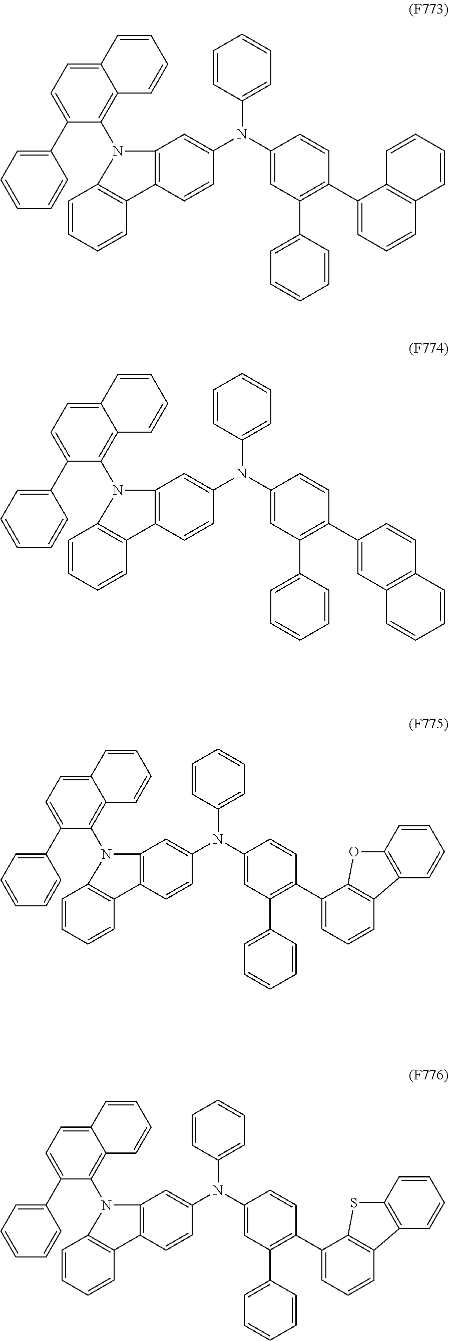 [SMILES: c1ccc(-c2cc(N(c3ccccc3)c3ccc4c5ccccc5n(-c5c(-c6ccccc6)ccc6ccccc56)c4c3)ccc2-c2cccc3ccccc23)cc1 ; c1ccc(-c2cc(N(c3ccccc3)c3ccc4c5ccccc5n(-c5c(-c6ccccc6)ccc6ccccc56)c4c3)ccc2-c2ccc3ccccc3c2)cc1 ; c1ccc(-c2cc(N(c3ccccc3)c3ccc4c5ccccc5n(-c5c(-c6ccccc6)ccc6ccccc56)c4c3)ccc2-c2cccc3c2oc2ccccc23)cc1 ; c1ccc(-c2cc(N(c3ccccc3)c3ccc4c5ccccc5n(-c5c(-c6ccccc6)ccc6ccccc56)c4c3)ccc2-c2cccc3c2sc2ccccc23)cc1]

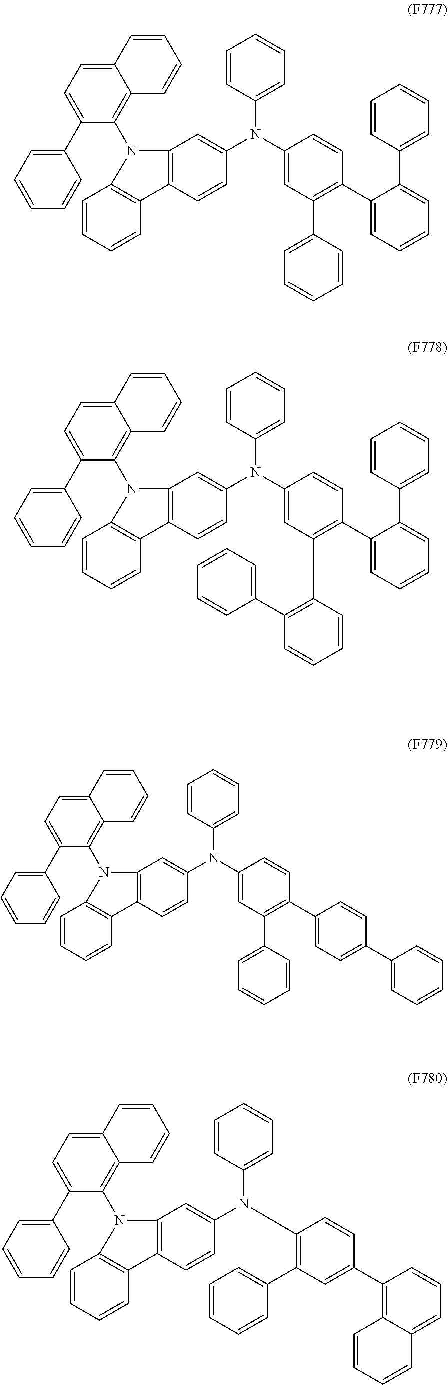 [SMILES: c1ccc(-c2ccccc2-c2ccc(N(c3ccccc3)c3ccc4c5ccccc5n(-c5c(-c6ccccc6)ccc6ccccc56)c4c3)cc2-c2ccccc2)cc1 ; c1ccc(-c2ccccc2-c2ccc(N(c3ccccc3)c3ccc4c5ccccc5n(-c5c(-c6ccccc6)ccc6ccccc56)c4c3)cc2-c2ccccc2-c2ccccc2)cc1 ; c1ccc(-c2ccc(-c3ccc(N(c4ccccc4)c4ccc5c6ccccc6n(-c6c(-c7ccccc7)ccc7ccccc67)c5c4)cc3-c3ccccc3)cc2)cc1 ; c1ccc(-c2cc(-c3cccc4ccccc34)ccc2N(c2ccccc2)c2ccc3c4ccccc4n(-c4c(-c5ccccc5)ccc5ccccc45)c3c2)cc1]

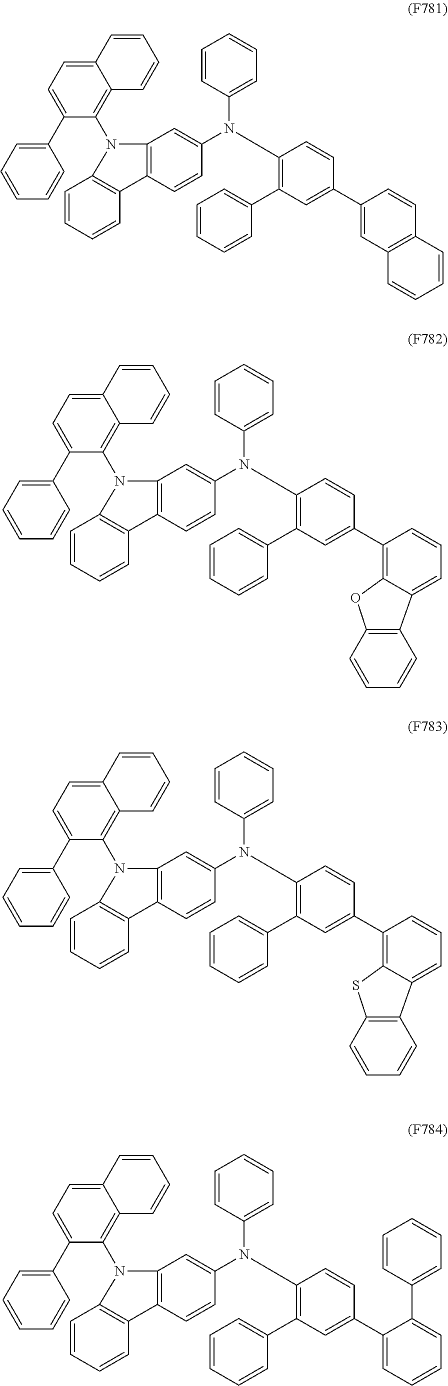 [SMILES: c1ccc(-c2cc(-c3ccc4ccccc4c3)ccc2N(c2ccccc2)c2ccc3c4ccccc4n(-c4c(-c5ccccc5)ccc5ccccc45)c3c2)cc1 ; c1ccc(-c2cc(-c3cccc4c3oc3ccccc34)ccc2N(c2ccccc2)c2ccc3c4ccccc4n(-c4c(-c5ccccc5)ccc5ccccc45)c3c2)cc1 ; c1ccc(-c2cc(-c3cccc4c3sc3ccccc34)ccc2N(c2ccccc2)c2ccc3c4ccccc4n(-c4c(-c5ccccc5)ccc5ccccc45)c3c2)cc1 ; c1ccc(-c2ccccc2-c2ccc(N(c3ccccc3)c3ccc4c5ccccc5n(-c5c(-c6ccccc6)ccc6ccccc56)c4c3)c(-c3ccccc3)c2)cc1]

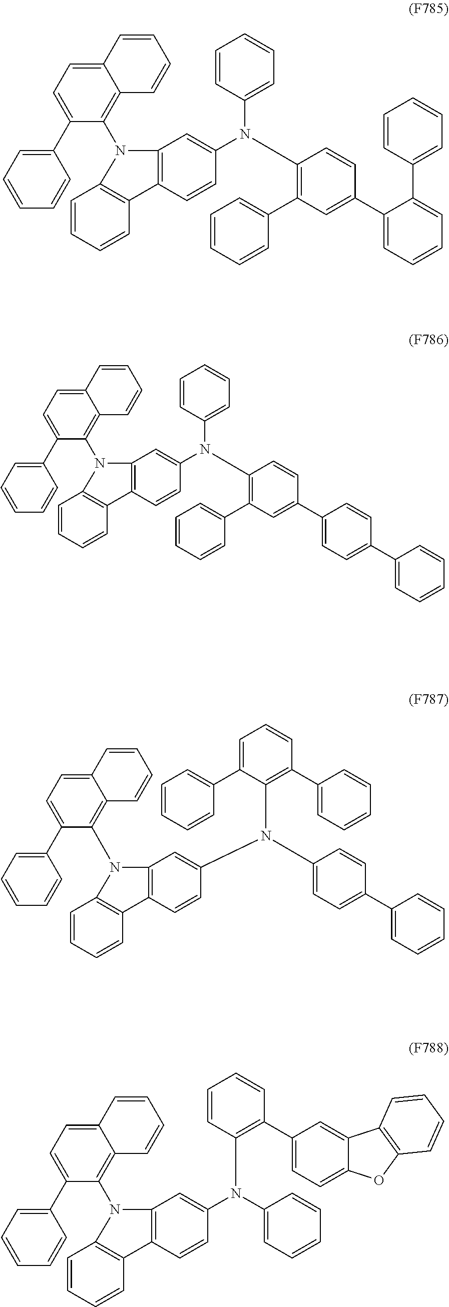 [SMILES: c1ccc(-c2ccccc2-c2ccc(N(c3ccccc3)c3ccc4c5ccccc5n(-c5c(-c6ccccc6)ccc6ccccc56)c4c3)c(-c3ccccc3)c2)cc1 ; c1ccc(-c2ccc(-c3ccc(N(c4ccccc4)c4ccc5c6ccccc6n(-c6c(-c7ccccc7)ccc7ccccc67)c5c4)c(-c4ccccc4)c3)cc2)cc1 ; c1ccc(-c2ccc(N(c3ccc4c5ccccc5n(-c5c(-c6ccccc6)ccc6ccccc56)c4c3)c3c(-c4ccccc4)cccc3-c3ccccc3)cc2)cc1 ; c1ccc(-c2ccc3ccccc3c2-n2c3ccccc3c3ccc(N(c4ccccc4)c4ccccc4-c4ccc5oc6ccccc6c5c4)cc32)cc1]

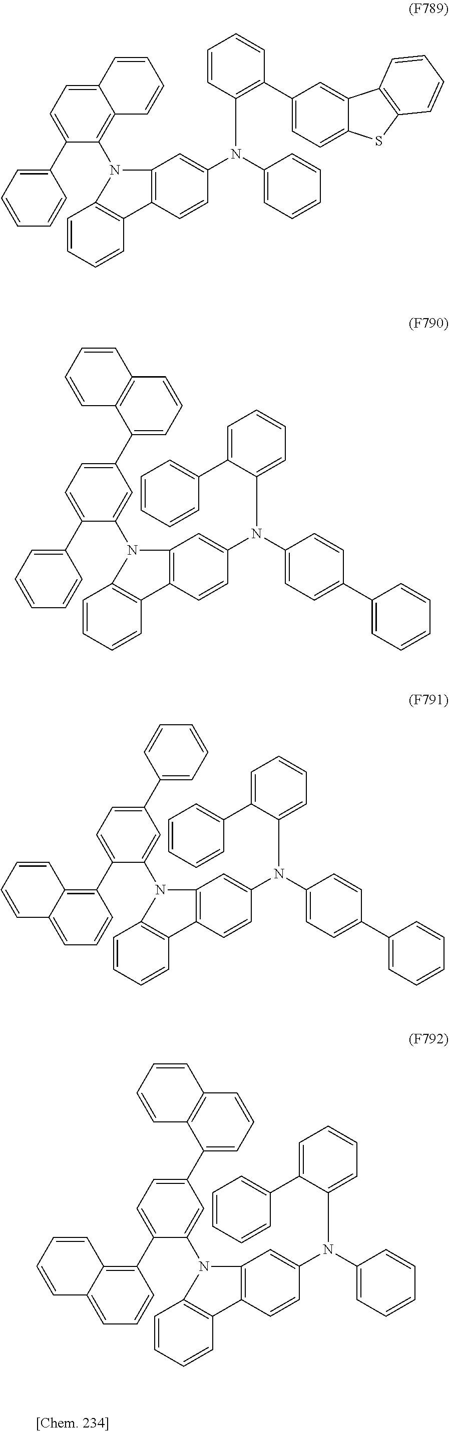 [SMILES: c1ccc(-c2ccc3ccccc3c2-n2c3ccccc3c3ccc(N(c4ccccc4)c4ccccc4-c4ccc5sc6ccccc6c5c4)cc32)cc1 ; c1ccc(-c2ccc(N(c3ccc4c5ccccc5n(-c5cc(-c6cccc7ccccc67)ccc5-c5ccccc5)c4c3)c3ccccc3-c3ccccc3)cc2)cc1 ; c1ccc(-c2ccc(N(c3ccc4c5ccccc5n(-c5cc(-c6ccccc6)ccc5-c5cccc6ccccc56)c4c3)c3ccccc3-c3ccccc3)cc2)cc1 ; c1ccc(-c2ccccc2N(c2ccccc2)c2ccc3c4ccccc4n(-c4cc(-c5cccc6ccccc56)ccc4-c4cccc5ccccc45)c3c2)cc1]

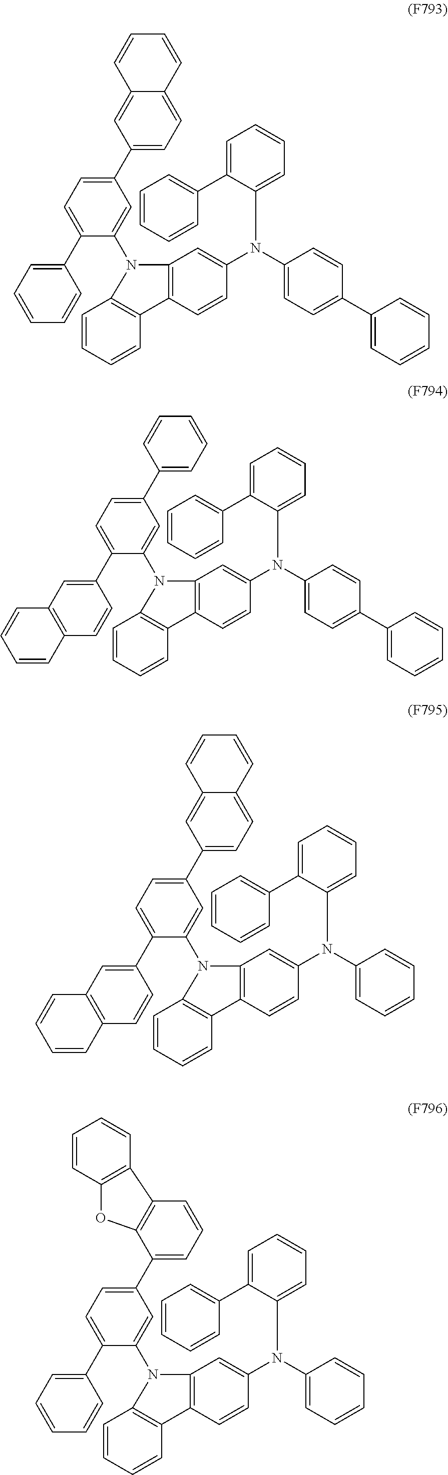 [SMILES: c1ccc(-c2ccc(N(c3ccc4c5ccccc5n(-c5cc(-c6ccc7ccccc7c6)ccc5-c5ccccc5)c4c3)c3ccccc3-c3ccccc3)cc2)cc1 ; c1ccc(-c2ccc(N(c3ccc4c5ccccc5n(-c5cc(-c6ccccc6)ccc5-c5ccc6ccccc6c5)c4c3)c3ccccc3-c3ccccc3)cc2)cc1 ; c1ccc(-c2ccccc2N(c2ccccc2)c2ccc3c4ccccc4n(-c4cc(-c5ccc6ccccc6c5)ccc4-c4ccc5ccccc5c4)c3c2)cc1 ; c1ccc(-c2ccccc2N(c2ccccc2)c2ccc3c4ccccc4n(-c4cc(-c5cccc6c5oc5ccccc56)ccc4-c4ccccc4)c3c2)cc1]

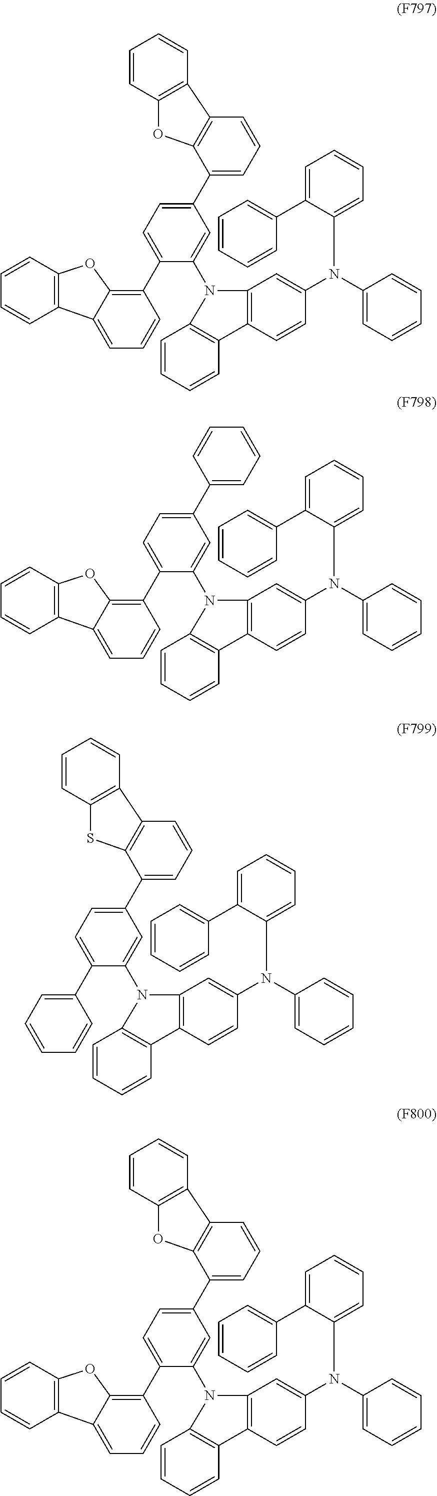

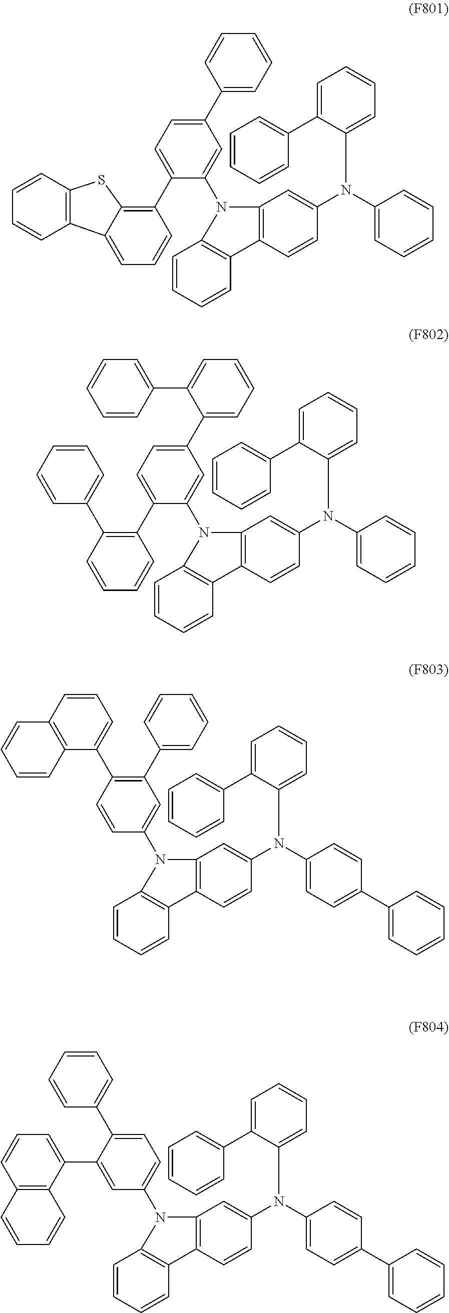 [SMILES: c1ccc(-c2ccc(-c3cccc4c3sc3ccccc34)c(-n3c4ccccc4c4ccc(N(c5ccccc5)c5ccccc5-c5ccccc5)cc43)c2)cc1 ; c1ccc(-c2ccccc2-c2ccc(-c3ccccc3-c3ccccc3)c(-n3c4ccccc4c4ccc(N(c5ccccc5)c5ccccc5-c5ccccc5)cc43)c2)cc1 ; c1ccc(-c2ccc(N(c3ccc4c5ccccc5n(-c5ccc(-c6cccc7ccccc67)c(-c6ccccc6)c5)c4c3)c3ccccc3-c3ccccc3)cc2)cc1 ; c1ccc(-c2ccc(N(c3ccc4c5ccccc5n(-c5ccc(-c6ccccc6)c(-c6cccc7ccccc67)c5)c4c3)c3ccccc3-c3ccccc3)cc2)cc1]

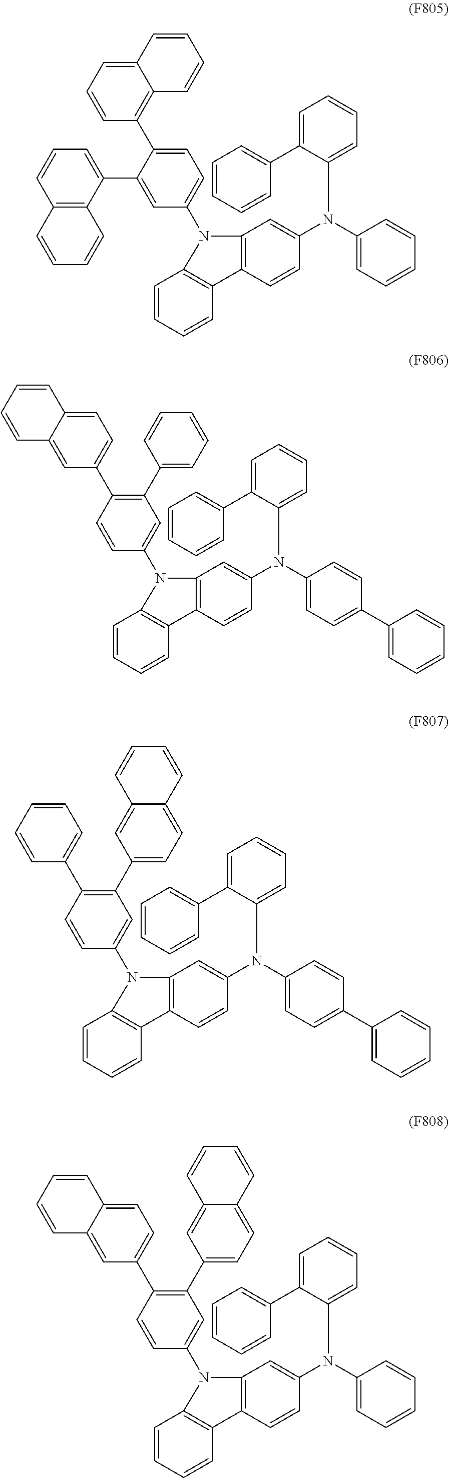 [SMILES: c1ccc(-c2ccccc2N(c2ccccc2)c2ccc3c4ccccc4n(-c4ccc(-c5cccc6ccccc56)c(-c5cccc6ccccc56)c4)c3c2)cc1 ; c1ccc(-c2ccc(N(c3ccc4c5ccccc5n(-c5ccc(-c6ccc7ccccc7c6)c(-c6ccccc6)c5)c4c3)c3ccccc3-c3ccccc3)cc2)cc1 ; c1ccc(-c2ccc(N(c3ccc4c5ccccc5n(-c5ccc(-c6ccccc6)c(-c6ccc7ccccc7c6)c5)c4c3)c3ccccc3-c3ccccc3)cc2)cc1 ; c1ccc(-c2ccccc2N(c2ccccc2)c2ccc3c4ccccc4n(-c4ccc(-c5ccc6ccccc6c5)c(-c5ccc6ccccc6c5)c4)c3c2)cc1]

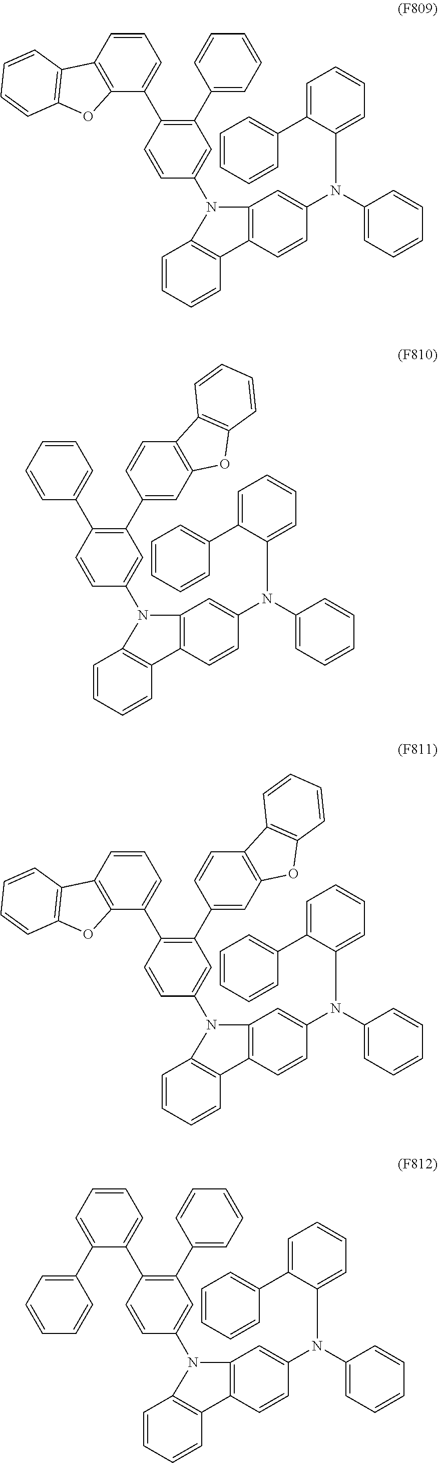 [SMILES: c1ccc(-c2cc(-n3c4ccccc4c4ccc(N(c5ccccc5)c5ccccc5-c5ccccc5)cc43)ccc2-c2cccc3c2oc2ccccc23)cc1 ; c1ccc(-c2ccc(-n3c4ccccc4c4ccc(N(c5ccccc5)c5ccccc5-c5ccccc5)cc43)cc2-c2ccc3c(c2)oc2ccccc23)cc1 ; c1ccc(-c2ccccc2N(c2ccccc2)c2ccc3c4ccccc4n(-c4ccc(-c5cccc6c5oc5ccccc56)c(-c5ccc6c(c5)oc5ccccc56)c4)c3c2)cc1 ; c1ccc(-c2ccccc2-c2ccc(-n3c4ccccc4c4ccc(N(c5ccccc5)c5ccccc5-c5ccccc5)cc43)cc2-c2ccccc2)cc1]

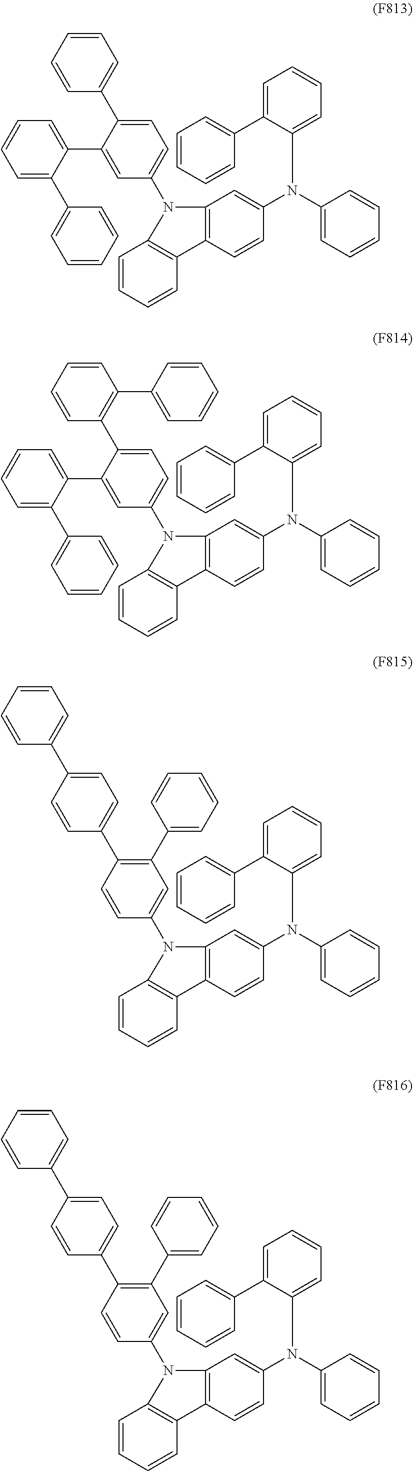 [SMILES: c1ccc(-c2ccccc2-c2cc(-n3c4ccccc4c4ccc(N(c5ccccc5)c5ccccc5-c5ccccc5)cc43)ccc2-c2ccccc2)cc1 ; c1ccc(-c2ccccc2-c2ccc(-n3c4ccccc4c4ccc(N(c5ccccc5)c5ccccc5-c5ccccc5)cc43)cc2-c2ccccc2-c2ccccc2)cc1 ; c1ccc(-c2ccc(-c3ccc(-n4c5ccccc5c5ccc(N(c6ccccc6)c6ccccc6-c6ccccc6)cc54)cc3-c3ccccc3)cc2)cc1 ; c1ccc(-c2ccc(-c3ccc(-n4c5ccccc5c5ccc(N(c6ccccc6)c6ccccc6-c6ccccc6)cc54)cc3-c3ccccc3)cc2)cc1]

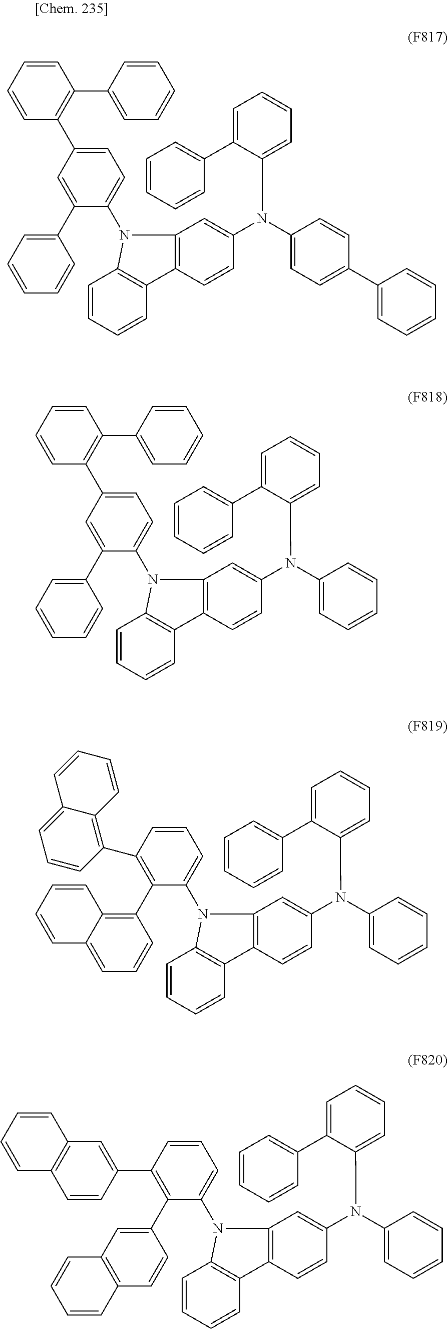 [SMILES: c1ccc(-c2ccc(N(c3ccc4c5ccccc5n(-c5ccc(-c6ccccc6-c6ccccc6)cc5-c5ccccc5)c4c3)c3ccccc3-c3ccccc3)cc2)cc1 ; c1ccc(-c2ccccc2-c2ccc(-n3c4ccccc4c4ccc(N(c5ccccc5)c5ccccc5-c5ccccc5)cc43)c(-c3ccccc3)c2)cc1 ; c1ccc(-c2ccccc2N(c2ccccc2)c2ccc3c4ccccc4n(-c4cccc(-c5cccc6ccccc56)c4-c4cccc5ccccc45)c3c2)cc1 ; c1ccc(-c2ccccc2N(c2ccccc2)c2ccc3c4ccccc4n(-c4cccc(-c5ccc6ccccc6c5)c4-c4ccc5ccccc5c4)c3c2)cc1]

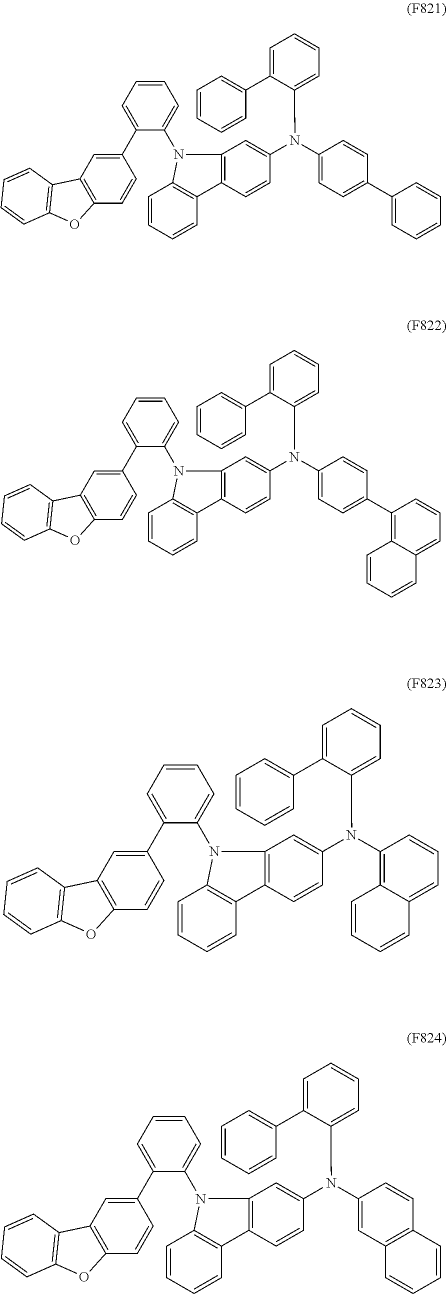 [SMILES: c1ccc(-c2ccc(N(c3ccc4c5ccccc5n(-c5ccccc5-c5ccc6oc7ccccc7c6c5)c4c3)c3ccccc3-c3ccccc3)cc2)cc1 ; c1ccc(-c2ccccc2N(c2ccc(-c3cccc4ccccc34)cc2)c2ccc3c4ccccc4n(-c4ccccc4-c4ccc5oc6ccccc6c5c4)c3c2)cc1 ; c1ccc(-c2ccccc2N(c2ccc3c4ccccc4n(-c4ccccc4-c4ccc5oc6ccccc6c5c4)c3c2)c2cccc3ccccc23)cc1 ; c1ccc(-c2ccccc2N(c2ccc3ccccc3c2)c2ccc3c4ccccc4n(-c4ccccc4-c4ccc5oc6ccccc6c5c4)c3c2)cc1]

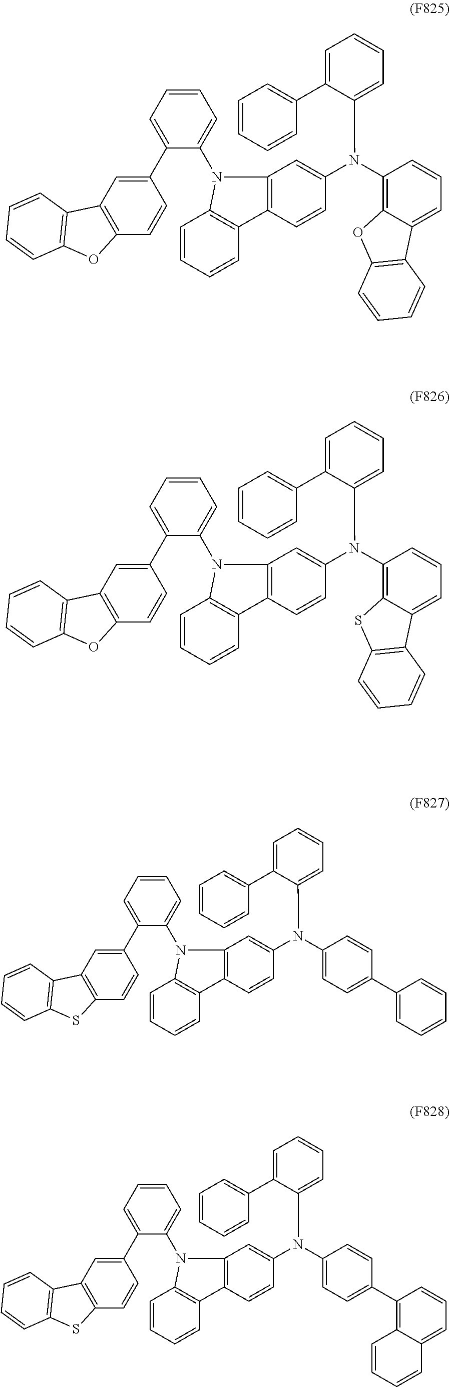 [SMILES: c1ccc(-c2ccccc2N(c2ccc3c4ccccc4n(-c4ccccc4-c4ccc5oc6ccccc6c5c4)c3c2)c2cccc3c2oc2ccccc23)cc1 ; c1ccc(-c2ccccc2N(c2ccc3c4ccccc4n(-c4ccccc4-c4ccc5oc6ccccc6c5c4)c3c2)c2cccc3c2sc2ccccc23)cc1 ; c1ccc(-c2ccc(N(c3ccc4c5ccccc5n(-c5ccccc5-c5ccc6sc7ccccc7c6c5)c4c3)c3ccccc3-c3ccccc3)cc2)cc1 ; c1ccc(-c2ccccc2N(c2ccc(-c3cccc4ccccc34)cc2)c2ccc3c4ccccc4n(-c4ccccc4-c4ccc5sc6ccccc6c5c4)c3c2)cc1]

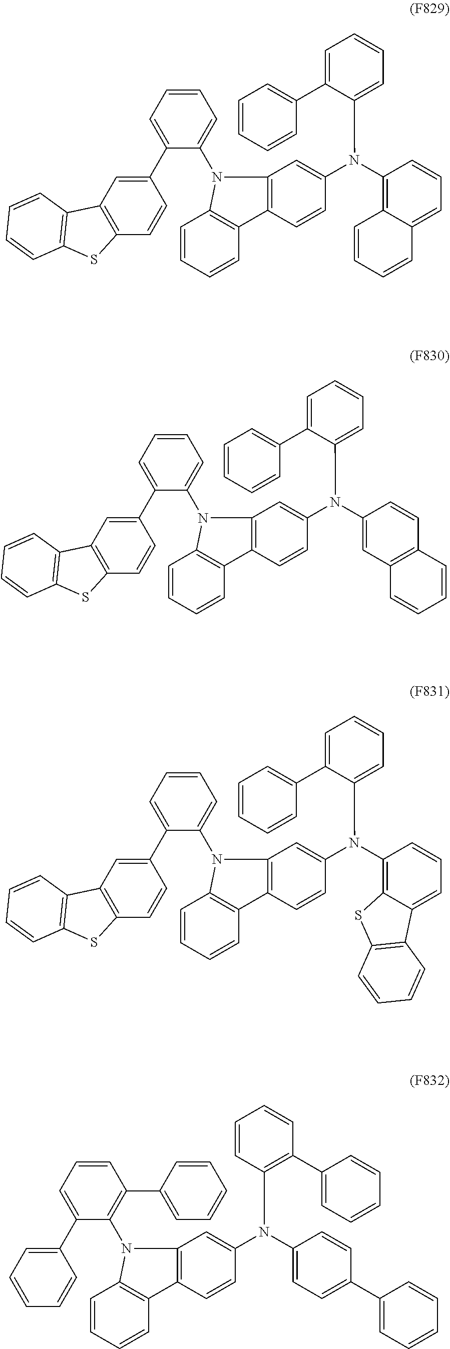 [SMILES: c1ccc(-c2ccccc2N(c2ccc3c4ccccc4n(-c4ccccc4-c4ccc5sc6ccccc6c5c4)c3c2)c2cccc3ccccc23)cc1 ; c1ccc(-c2ccccc2N(c2ccc3ccccc3c2)c2ccc3c4ccccc4n(-c4ccccc4-c4ccc5sc6ccccc6c5c4)c3c2)cc1 ; c1ccc(-c2ccccc2N(c2ccc3c4ccccc4n(-c4ccccc4-c4ccc5sc6ccccc6c5c4)c3c2)c2cccc3c2sc2ccccc23)cc1 ; c1ccc(-c2ccc(N(c3ccc4c5ccccc5n(-c5c(-c6ccccc6)cccc5-c5ccccc5)c4c3)c3ccccc3-c3ccccc3)cc2)cc1]

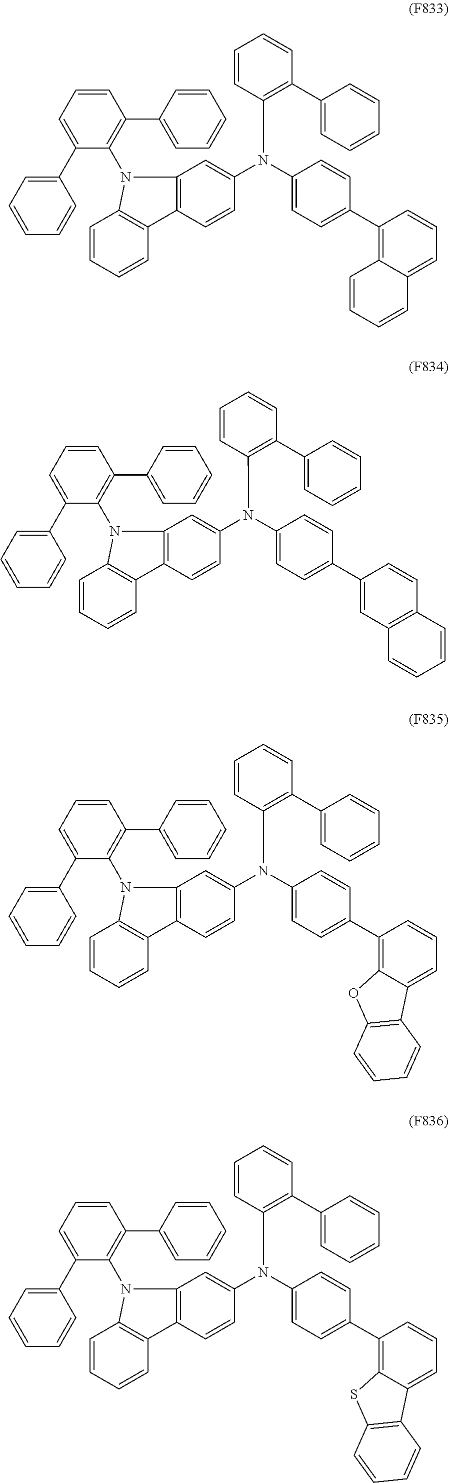 [SMILES: c1ccc(-c2ccccc2N(c2ccc(-c3cccc4ccccc34)cc2)c2ccc3c4ccccc4n(-c4c(-c5ccccc5)cccc4-c4ccccc4)c3c2)cc1 ; c1ccc(-c2ccccc2N(c2ccc(-c3ccc4ccccc4c3)cc2)c2ccc3c4ccccc4n(-c4c(-c5ccccc5)cccc4-c4ccccc4)c3c2)cc1 ; c1ccc(-c2ccccc2N(c2ccc(-c3cccc4c3oc3ccccc34)cc2)c2ccc3c4ccccc4n(-c4c(-c5ccccc5)cccc4-c4ccccc4)c3c2)cc1 ; c1ccc(-c2ccccc2N(c2ccc(-c3cccc4c3sc3ccccc34)cc2)c2ccc3c4ccccc4n(-c4c(-c5ccccc5)cccc4-c4ccccc4)c3c2)cc1]

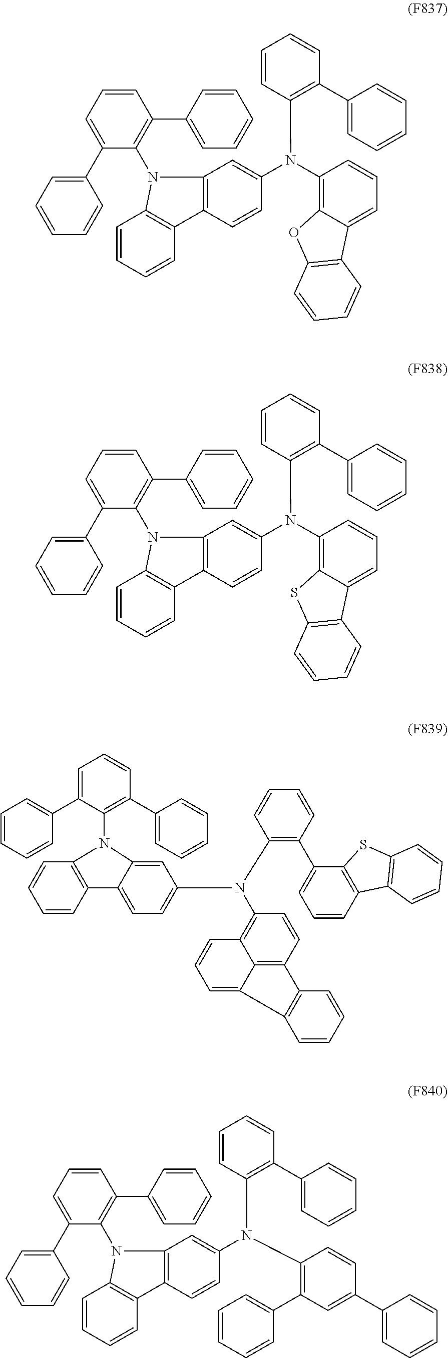 [SMILES: c1ccc(-c2ccccc2N(c2ccc3c4ccccc4n(-c4c(-c5ccccc5)cccc4-c4ccccc4)c3c2)c2cccc3c2oc2ccccc23)cc1 ; c1ccc(-c2ccccc2N(c2ccc3c4ccccc4n(-c4c(-c5ccccc5)cccc4-c4ccccc4)c3c2)c2cccc3c2sc2ccccc23)cc1 ; c1ccc(-c2cccc(-c3ccccc3)c2-n2c3ccccc3c3ccc(N(c4ccccc4-c4cccc5c4sc4ccccc45)c4ccc5c6c(cccc46)-c4ccccc4-5)cc32)cc1 ; c1ccc(-c2ccc(N(c3ccc4c5ccccc5n(-c5c(-c6ccccc6)cccc5-c5ccccc5)c4c3)c3ccccc3-c3ccccc3)c(-c3ccccc3)c2)cc1]

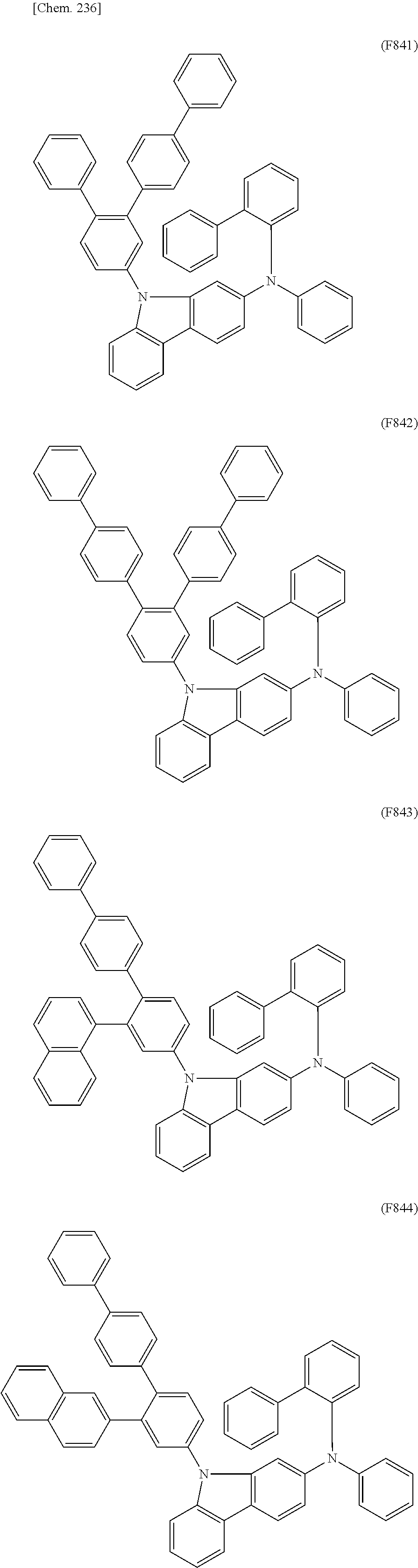 [SMILES: c1ccc(-c2ccc(-c3cc(-n4c5ccccc5c5ccc(N(c6ccccc6)c6ccccc6-c6ccccc6)cc54)ccc3-c3ccccc3)cc2)cc1 ; c1ccc(-c2ccc(-c3ccc(-n4c5ccccc5c5ccc(N(c6ccccc6)c6ccccc6-c6ccccc6)cc54)cc3-c3ccc(-c4ccccc4)cc3)cc2)cc1 ; c1ccc(-c2ccc(-c3ccc(-n4c5ccccc5c5ccc(N(c6ccccc6)c6ccccc6-c6ccccc6)cc54)cc3-c3cccc4ccccc34)cc2)cc1 ; c1ccc(-c2ccc(-c3ccc(-n4c5ccccc5c5ccc(N(c6ccccc6)c6ccccc6-c6ccccc6)cc54)cc3-c3ccc4ccccc4c3)cc2)cc1]

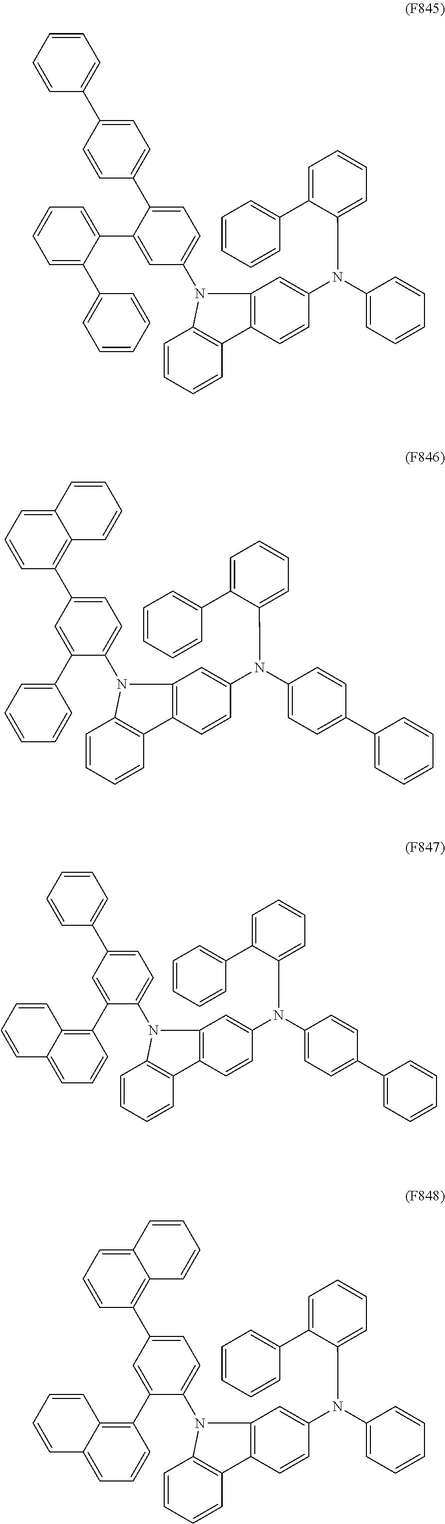 [SMILES: c1ccc(-c2ccc(-c3ccc(-n4c5ccccc5c5ccc(N(c6ccccc6)c6ccccc6-c6ccccc6)cc54)cc3-c3ccccc3-c3ccccc3)cc2)cc1 ; c1ccc(-c2ccc(N(c3ccc4c5ccccc5n(-c5ccc(-c6cccc7ccccc67)cc5-c5ccccc5)c4c3)c3ccccc3-c3ccccc3)cc2)cc1 ; c1ccc(-c2ccc(N(c3ccc4c5ccccc5n(-c5ccc(-c6ccccc6)cc5-c5cccc6ccccc56)c4c3)c3ccccc3-c3ccccc3)cc2)cc1 ; c1ccc(-c2ccccc2N(c2ccccc2)c2ccc3c4ccccc4n(-c4ccc(-c5cccc6ccccc56)cc4-c4cccc5ccccc45)c3c2)cc1]

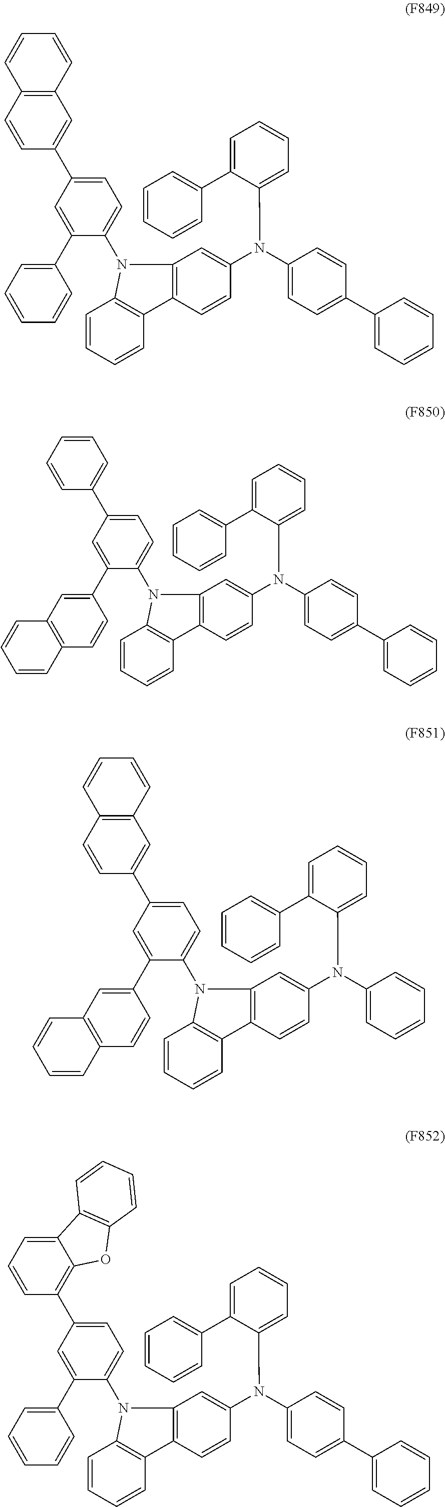 [SMILES: c1ccc(-c2ccc(N(c3ccc4c5ccccc5n(-c5ccc(-c6ccc7ccccc7c6)cc5-c5ccccc5)c4c3)c3ccccc3-c3ccccc3)cc2)cc1 ; c1ccc(-c2ccc(N(c3ccc4c5ccccc5n(-c5ccc(-c6ccccc6)cc5-c5ccc6ccccc6c5)c4c3)c3ccccc3-c3ccccc3)cc2)cc1 ; c1ccc(-c2ccccc2N(c2ccccc2)c2ccc3c4ccccc4n(-c4ccc(-c5ccc6ccccc6c5)cc4-c4ccc5ccccc5c4)c3c2)cc1 ; c1ccc(-c2ccc(N(c3ccc4c5ccccc5n(-c5ccc(-c6cccc7c6oc6ccccc67)cc5-c5ccccc5)c4c3)c3ccccc3-c3ccccc3)cc2)cc1]

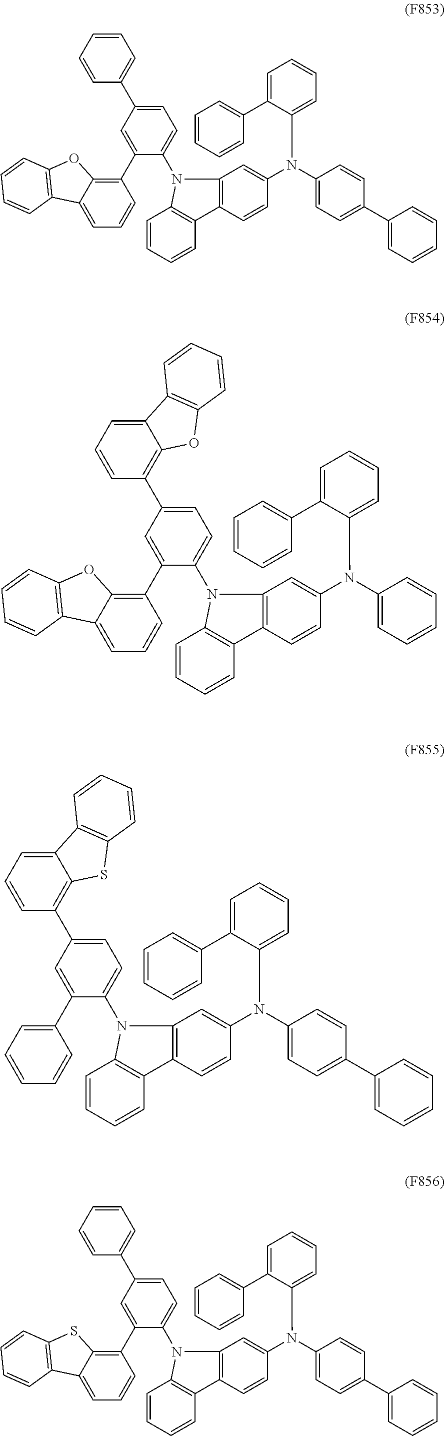 [SMILES: c1ccc(-c2ccc(N(c3ccc4c5ccccc5n(-c5ccc(-c6ccccc6)cc5-c5cccc6c5oc5ccccc56)c4c3)c3ccccc3-c3ccccc3)cc2)cc1 ; c1ccc(-c2ccccc2N(c2ccccc2)c2ccc3c4ccccc4n(-c4ccc(-c5cccc6c5oc5ccccc56)cc4-c4cccc5c4oc4ccccc45)c3c2)cc1 ; c1ccc(-c2ccc(N(c3ccc4c5ccccc5n(-c5ccc(-c6cccc7c6sc6ccccc67)cc5-c5ccccc5)c4c3)c3ccccc3-c3ccccc3)cc2)cc1 ; c1ccc(-c2ccc(N(c3ccc4c5ccccc5n(-c5ccc(-c6ccccc6)cc5-c5cccc6c5sc5ccccc56)c4c3)c3ccccc3-c3ccccc3)cc2)cc1]

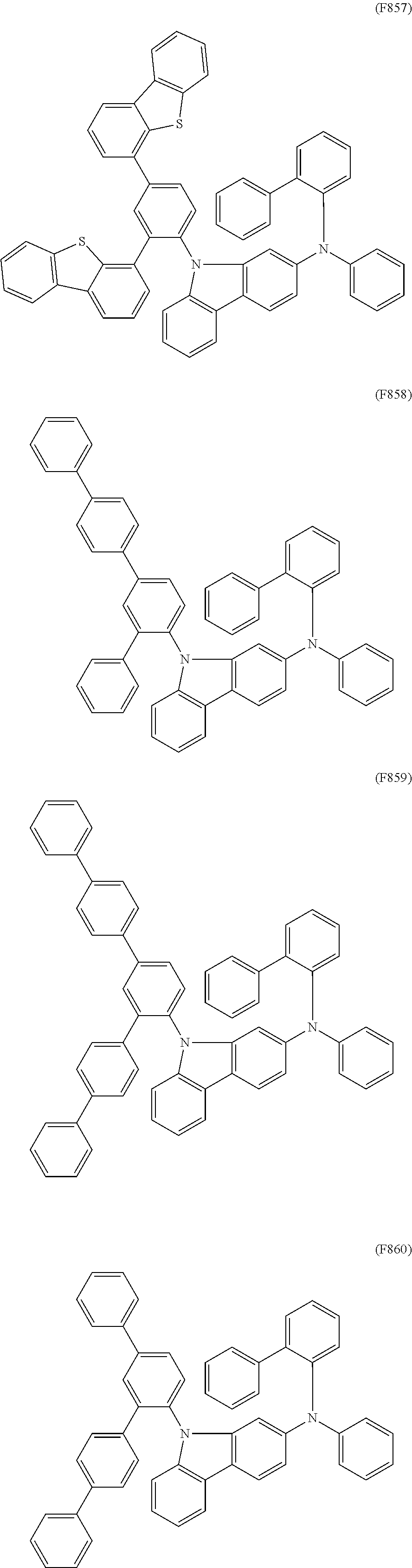 [SMILES: c1ccc(-c2ccccc2N(c2ccccc2)c2ccc3c4ccccc4n(-c4ccc(-c5cccc6c5sc5ccccc56)cc4-c4cccc5c4sc4ccccc45)c3c2)cc1 ; c1ccc(-c2ccc(-c3ccc(-n4c5ccccc5c5ccc(N(c6ccccc6)c6ccccc6-c6ccccc6)cc54)c(-c4ccccc4)c3)cc2)cc1 ; c1ccc(-c2ccc(-c3ccc(-n4c5ccccc5c5ccc(N(c6ccccc6)c6ccccc6-c6ccccc6)cc54)c(-c4ccc(-c5ccccc5)cc4)c3)cc2)cc1 ; c1ccc(-c2ccc(-c3cc(-c4ccccc4)ccc3-n3c4ccccc4c4ccc(N(c5ccccc5)c5ccccc5-c5ccccc5)cc43)cc2)cc1]

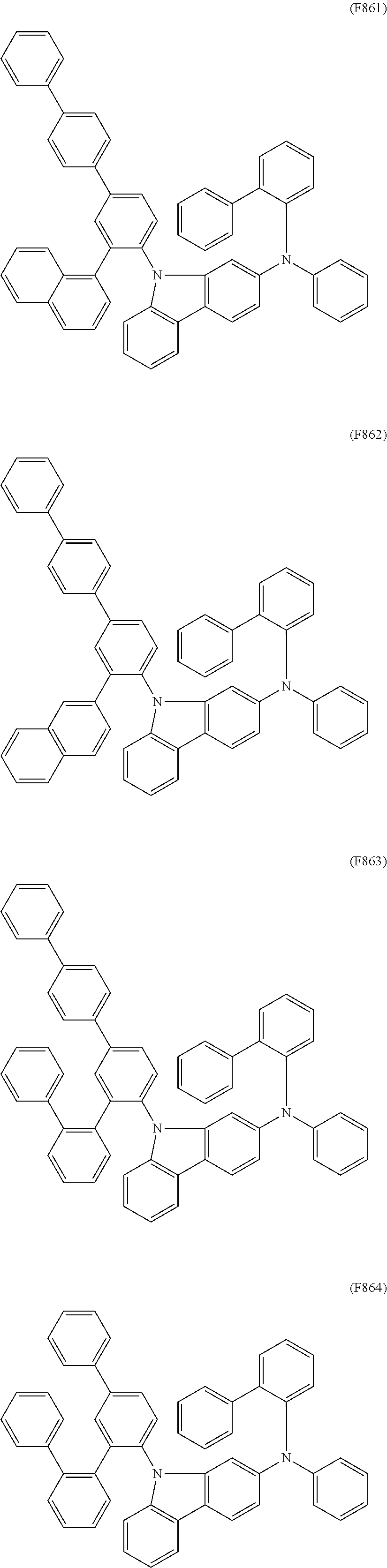 [SMILES: c1ccc(-c2ccc(-c3ccc(-n4c5ccccc5c5ccc(N(c6ccccc6)c6ccccc6-c6ccccc6)cc54)c(-c4cccc5ccccc45)c3)cc2)cc1 ; c1ccc(-c2ccc(-c3ccc(-n4c5ccccc5c5ccc(N(c6ccccc6)c6ccccc6-c6ccccc6)cc54)c(-c4ccc5ccccc5c4)c3)cc2)cc1 ; c1ccc(-c2ccc(-c3ccc(-n4c5ccccc5c5ccc(N(c6ccccc6)c6ccccc6-c6ccccc6)cc54)c(-c4ccccc4-c4ccccc4)c3)cc2)cc1 ; c1ccc(-c2ccc(-n3c4ccccc4c4ccc(N(c5ccccc5)c5ccccc5-c5ccccc5)cc43)c(-c3ccccc3-c3ccccc3)c2)cc1]

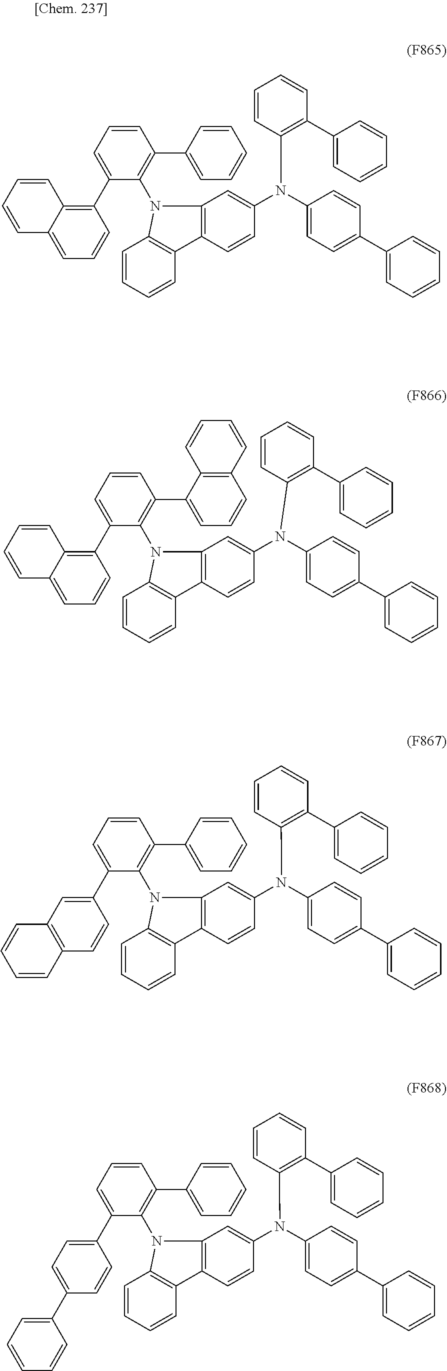 [SMILES: c1ccc(-c2ccc(N(c3ccc4c5ccccc5n(-c5c(-c6ccccc6)cccc5-c5cccc6ccccc56)c4c3)c3ccccc3-c3ccccc3)cc2)cc1 ; c1ccc(-c2ccc(N(c3ccc4c5ccccc5n(-c5c(-c6cccc7ccccc67)cccc5-c5cccc6ccccc56)c4c3)c3ccccc3-c3ccccc3)cc2)cc1 ; c1ccc(-c2ccc(N(c3ccc4c5ccccc5n(-c5c(-c6ccccc6)cccc5-c5ccc6ccccc6c5)c4c3)c3ccccc3-c3ccccc3)cc2)cc1 ; c1ccc(-c2ccc(-c3cccc(-c4ccccc4)c3-n3c4ccccc4c4ccc(N(c5ccc(-c6ccccc6)cc5)c5ccccc5-c5ccccc5)cc43)cc2)cc1]

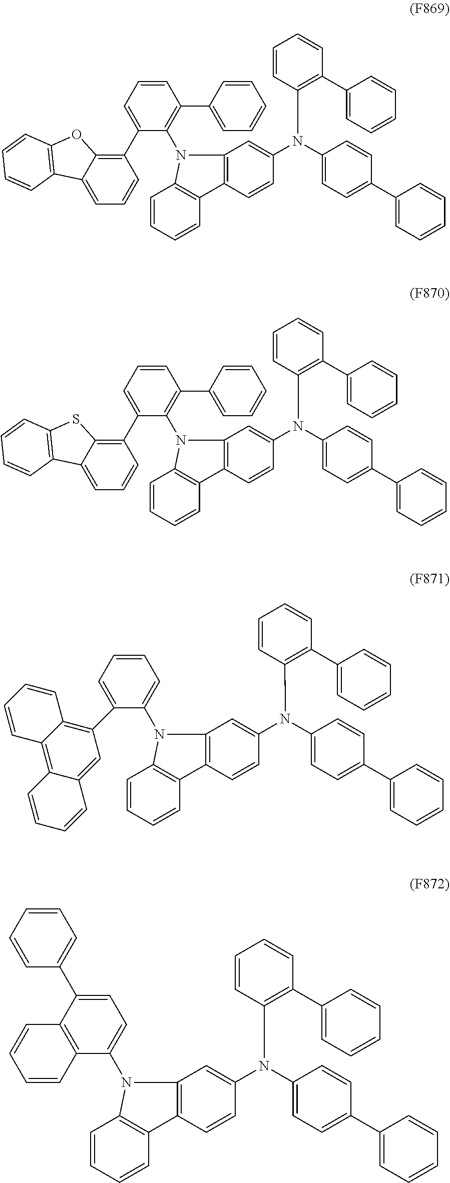 [SMILES: c1ccc(-c2ccc(N(c3ccc4c5ccccc5n(-c5c(-c6ccccc6)cccc5-c5cccc6c5oc5ccccc56)c4c3)c3ccccc3-c3ccccc3)cc2)cc1 ; c1ccc(-c2ccc(N(c3ccc4c5ccccc5n(-c5c(-c6ccccc6)cccc5-c5cccc6c5sc5ccccc56)c4c3)c3ccccc3-c3ccccc3)cc2)cc1 ; c1ccc(-c2ccc(N(c3ccc4c5ccccc5n(-c5ccccc5-c5cc6ccccc6c6ccccc56)c4c3)c3ccccc3-c3ccccc3)cc2)cc1 ; c1ccc(-c2ccc(N(c3ccc4c5ccccc5n(-c5ccc(-c6ccccc6)c6ccccc56)c4c3)c3ccccc3-c3ccccc3)cc2)cc1]

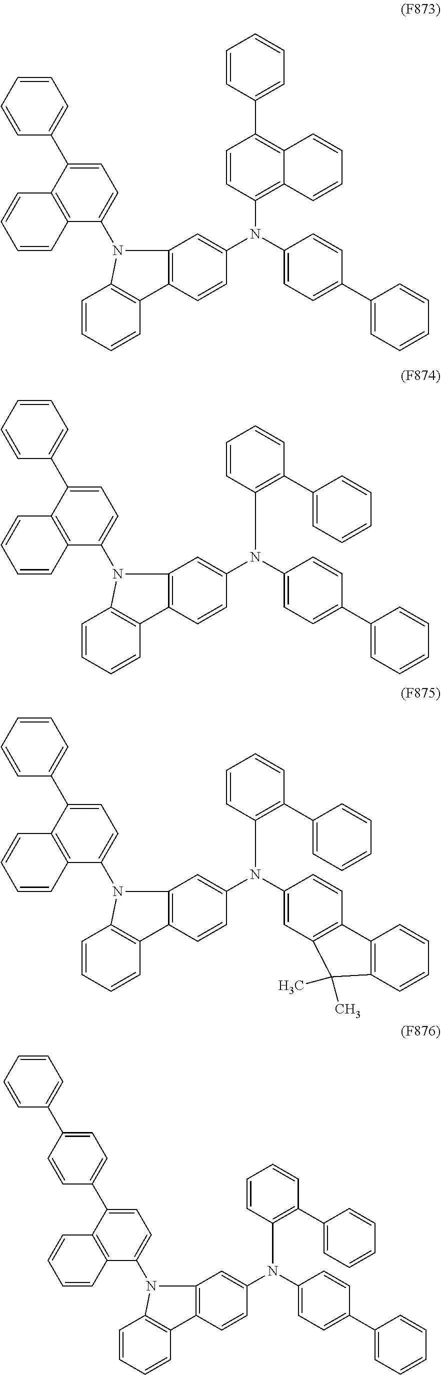 [SMILES: c1ccc(-c2ccc(N(c3ccc4c5ccccc5n(-c5ccc(-c6ccccc6)c6ccccc56)c4c3)c3ccc(-c4ccccc4)c4ccccc34)cc2)cc1 ; c1ccc(-c2ccc(N(c3ccc4c5ccccc5n(-c5ccc(-c6ccccc6)c6ccccc56)c4c3)c3ccccc3-c3ccccc3)cc2)cc1 ; CC1(C)c2ccccc2-c2ccc(N(c3ccc4c5ccccc5n(-c5ccc(-c6ccccc6)c6ccccc56)c4c3)c3ccccc3-c3ccccc3)cc21 ; c1ccc(-c2ccc(-c3ccc(-n4c5ccccc5c5ccc(N(c6ccc(-c7ccccc7)cc6)c6ccccc6-c6ccccc6)cc54)c4ccccc34)cc2)cc1]

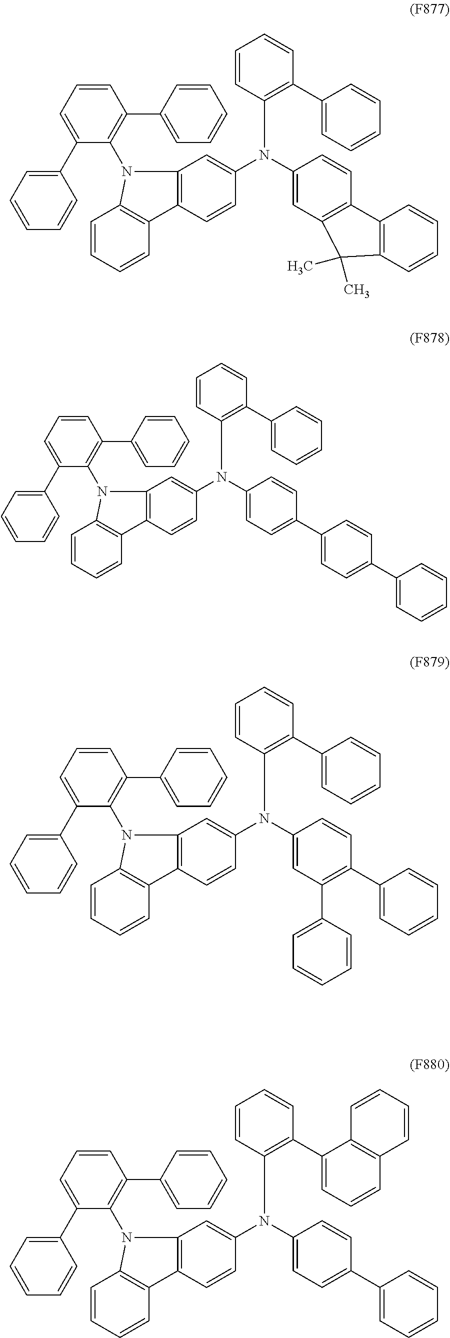 [SMILES: CC1(C)c2ccccc2-c2ccc(N(c3ccc4c5ccccc5n(-c5c(-c6ccccc6)cccc5-c5ccccc5)c4c3)c3ccccc3-c3ccccc3)cc21 ; c1ccc(-c2ccc(-c3ccc(N(c4ccc5c6ccccc6n(-c6c(-c7ccccc7)cccc6-c6ccccc6)c5c4)c4ccccc4-c4ccccc4)cc3)cc2)cc1 ; c1ccc(-c2ccc(N(c3ccc4c5ccccc5n(-c5c(-c6ccccc6)cccc5-c5ccccc5)c4c3)c3ccccc3-c3ccccc3)cc2-c2ccccc2)cc1 ; c1ccc(-c2ccc(N(c3ccc4c5ccccc5n(-c5c(-c6ccccc6)cccc5-c5ccccc5)c4c3)c3ccccc3-c3cccc4ccccc34)cc2)cc1]

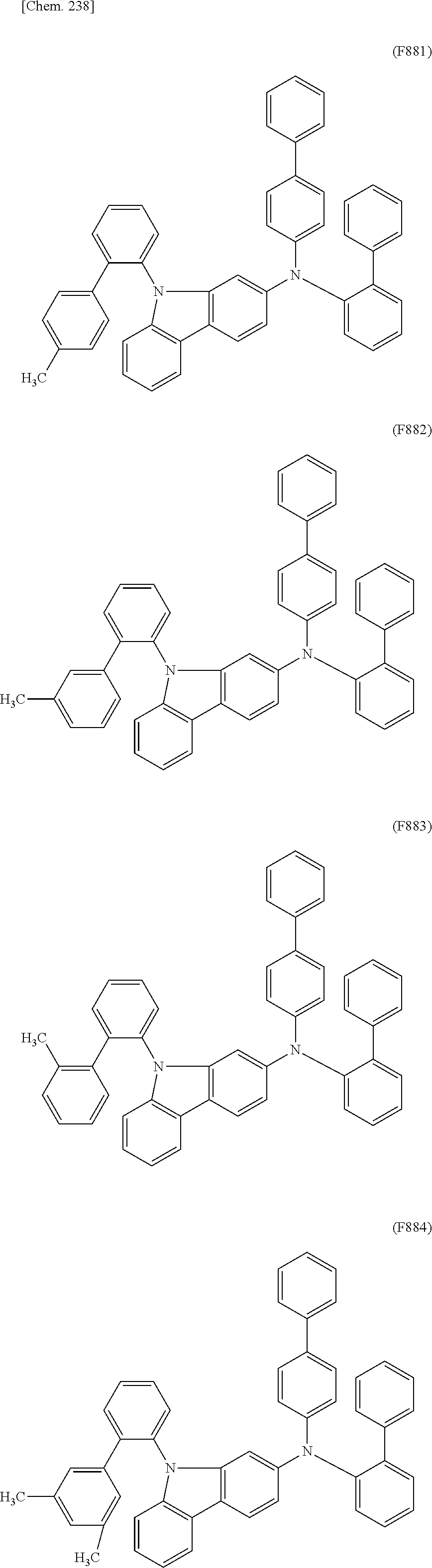

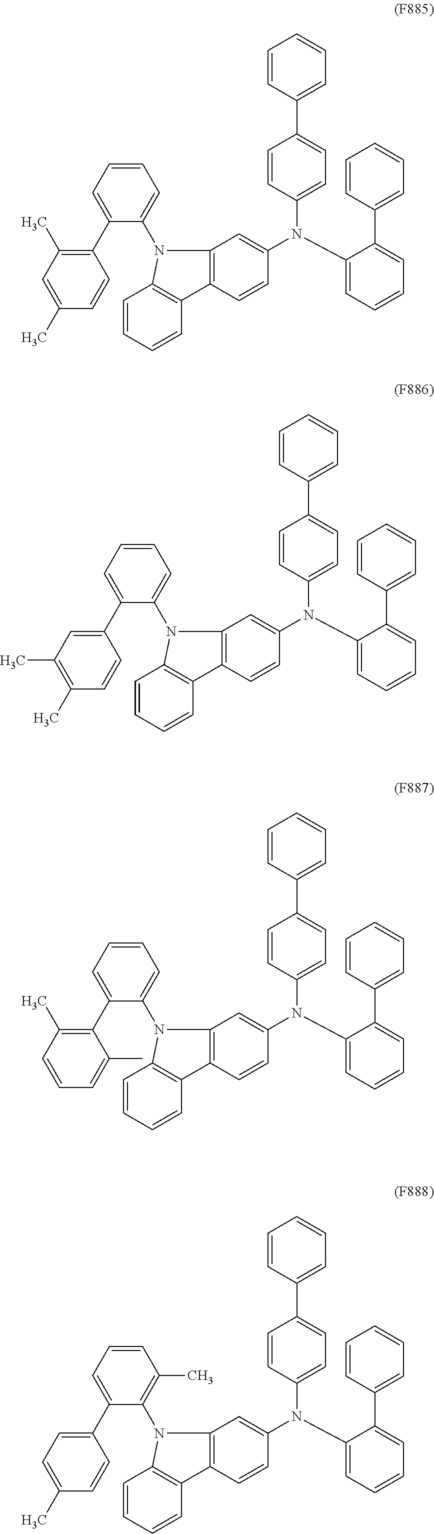 [SMILES: Cc1ccc(-c2ccccc2-n2c3ccccc3c3ccc(N(c4ccc(-c5ccccc5)cc4)c4ccccc4-c4ccccc4)cc32)c(C)c1 ; Cc1ccc(-c2ccccc2-n2c3ccccc3c3ccc(N(c4ccc(-c5ccccc5)cc4)c4ccccc4-c4ccccc4)cc32)cc1C ; Cc1cccc(C)c1-c1ccccc1-n1c2ccccc2c2ccc(N(c3ccc(-c4ccccc4)cc3)c3ccccc3-c3ccccc3)cc21 ; Cc1ccc(-c2cccc(C)c2-n2c3ccccc3c3ccc(N(c4ccc(-c5ccccc5)cc4)c4ccccc4-c4ccccc4)cc32)cc1]

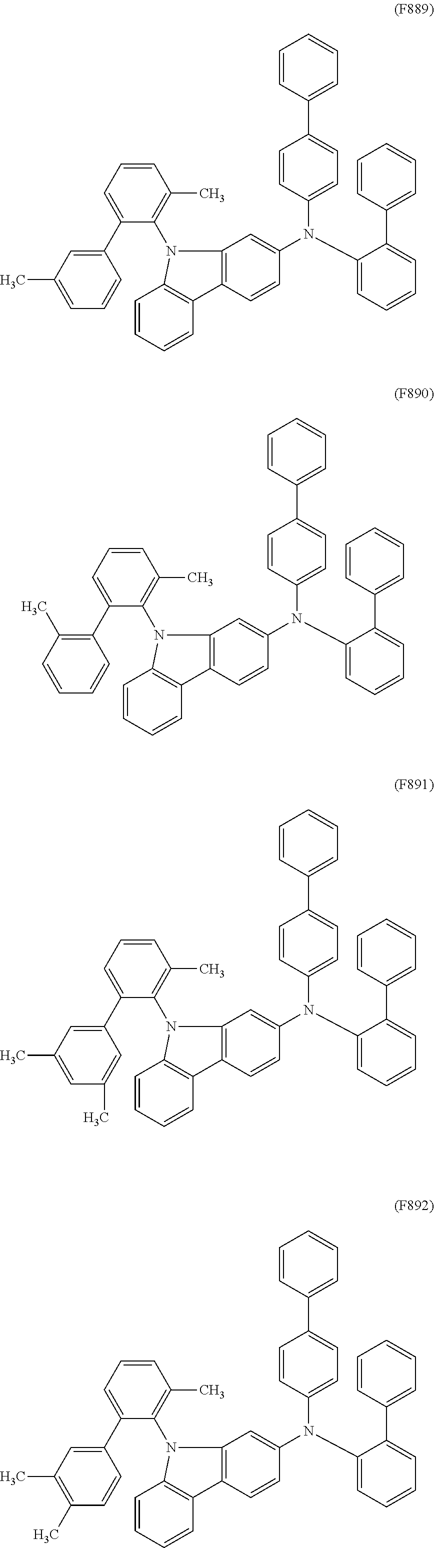 [SMILES: Cc1cccc(-c2cccc(C)c2-n2c3ccccc3c3ccc(N(c4ccc(-c5ccccc5)cc4)c4ccccc4-c4ccccc4)cc32)c1 ; Cc1ccccc1-c1cccc(C)c1-n1c2ccccc2c2ccc(N(c3ccc(-c4ccccc4)cc3)c3ccccc3-c3ccccc3)cc21 ; Cc1cc(C)cc(-c2cccc(C)c2-n2c3ccccc3c3ccc(N(c4ccc(-c5ccccc5)cc4)c4ccccc4-c4ccccc4)cc32)c1 ; Cc1ccc(-c2cccc(C)c2-n2c3ccccc3c3ccc(N(c4ccc(-c5ccccc5)cc4)c4ccccc4-c4ccccc4)cc32)cc1C]

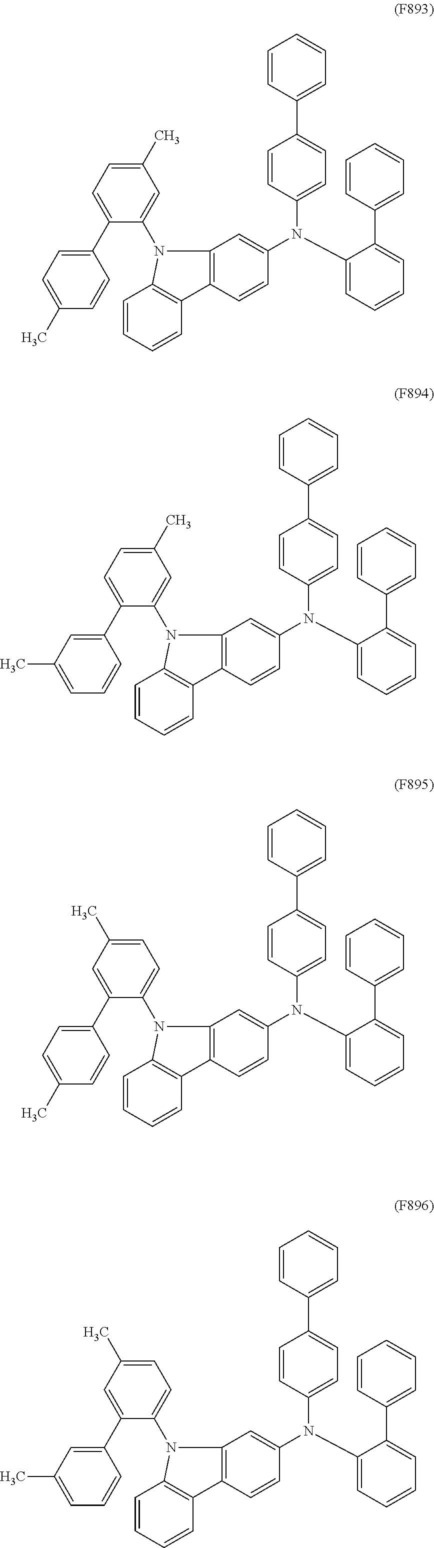 [SMILES: Cc1ccc(-c2ccc(C)cc2-n2c3ccccc3c3ccc(N(c4ccc(-c5ccccc5)cc4)c4ccccc4-c4ccccc4)cc32)cc1 ; Cc1cccc(-c2ccc(C)cc2-n2c3ccccc3c3ccc(N(c4ccc(-c5ccccc5)cc4)c4ccccc4-c4ccccc4)cc32)c1 ; Cc1ccc(-c2cc(C)ccc2-n2c3ccccc3c3ccc(N(c4ccc(-c5ccccc5)cc4)c4ccccc4-c4ccccc4)cc32)cc1 ; Cc1cccc(-c2cc(C)ccc2-n2c3ccccc3c3ccc(N(c4ccc(-c5ccccc5)cc4)c4ccccc4-c4ccccc4)cc32)c1]

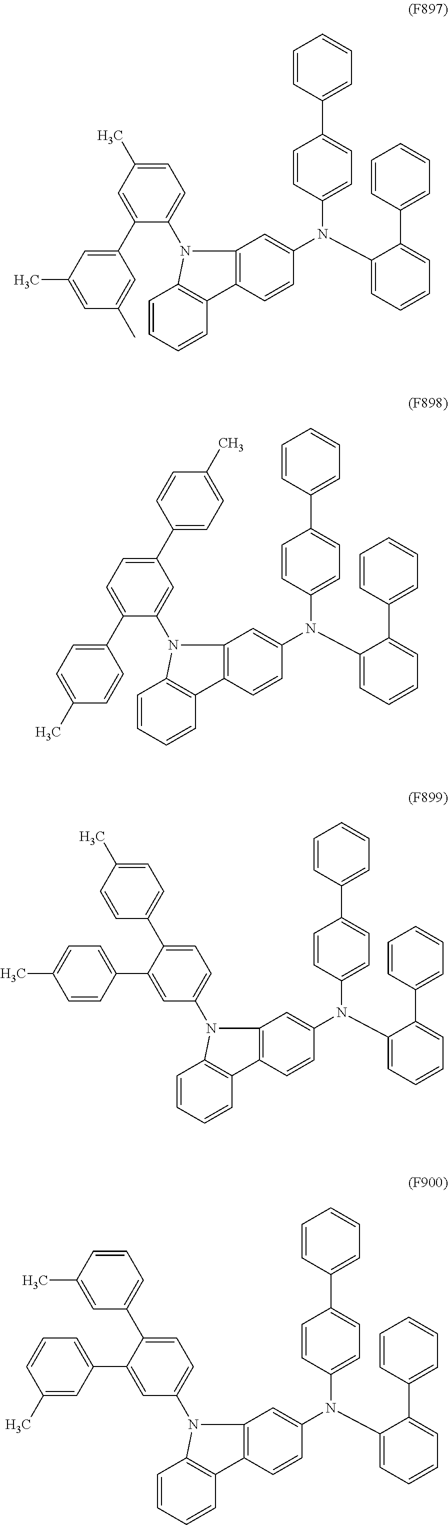 [SMILES: Cc1cc(C)cc(-c2cc(C)ccc2-n2c3ccccc3c3ccc(N(c4ccc(-c5ccccc5)cc4)c4ccccc4-c4ccccc4)cc32)c1 ; Cc1ccc(-c2ccc(-c3ccc(C)cc3)c(-n3c4ccccc4c4ccc(N(c5ccc(-c6ccccc6)cc5)c5ccccc5-c5ccccc5)cc43)c2)cc1 ; Cc1ccc(-c2ccc(-n3c4ccccc4c4ccc(N(c5ccc(-c6ccccc6)cc5)c5ccccc5-c5ccccc5)cc43)cc2-c2ccc(C)cc2)cc1 ; Cc1cccc(-c2ccc(-n3c4ccccc4c4ccc(N(c5ccc(-c6ccccc6)cc5)c5ccccc5-c5ccccc5)cc43)cc2-c2cccc(C)c2)c1]

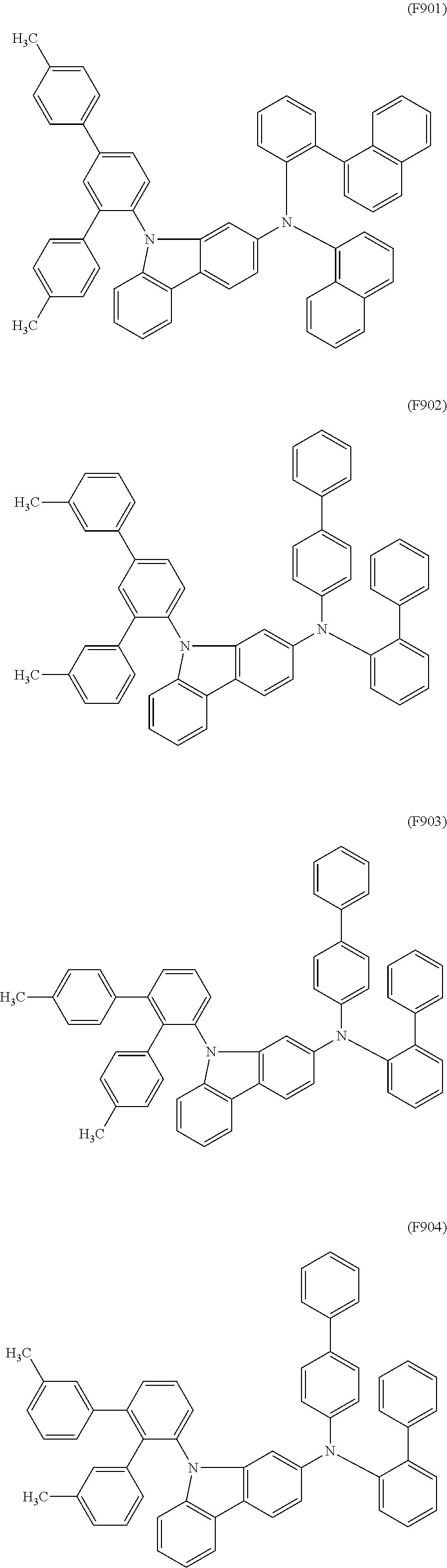 [SMILES: Cc1ccc(-c2ccc(-n3c4ccccc4c4ccc(N(c5ccccc5-c5cccc6ccccc56)c5cccc6ccccc56)cc43)c(-c3ccc(C)cc3)c2)cc1 ; Cc1cccc(-c2ccc(-n3c4ccccc4c4ccc(N(c5ccc(-c6ccccc6)cc5)c5ccccc5-c5ccccc5)cc43)c(-c3cccc(C)c3)c2)c1 ; Cc1ccc(-c2cccc(-n3c4ccccc4c4ccc(N(c5ccc(-c6ccccc6)cc5)c5ccccc5-c5ccccc5)cc43)c2-c2ccc(C)cc2)cc1 ; Cc1cccc(-c2cccc(-n3c4ccccc4c4ccc(N(c5ccc(-c6ccccc6)cc5)c5ccccc5-c5ccccc5)cc43)c2-c2cccc(C)c2)c1]

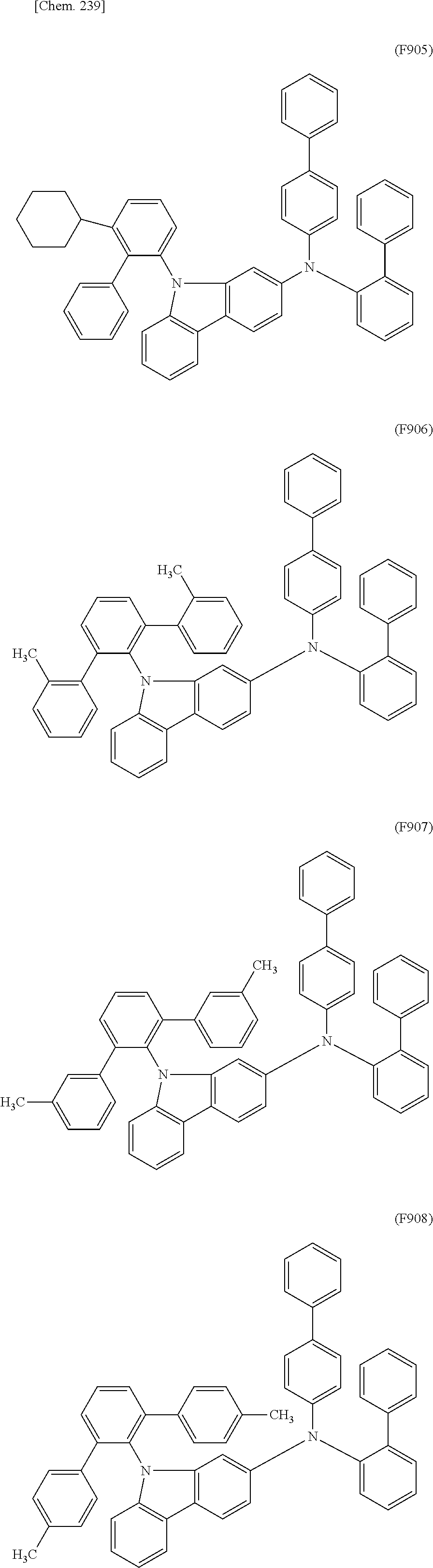

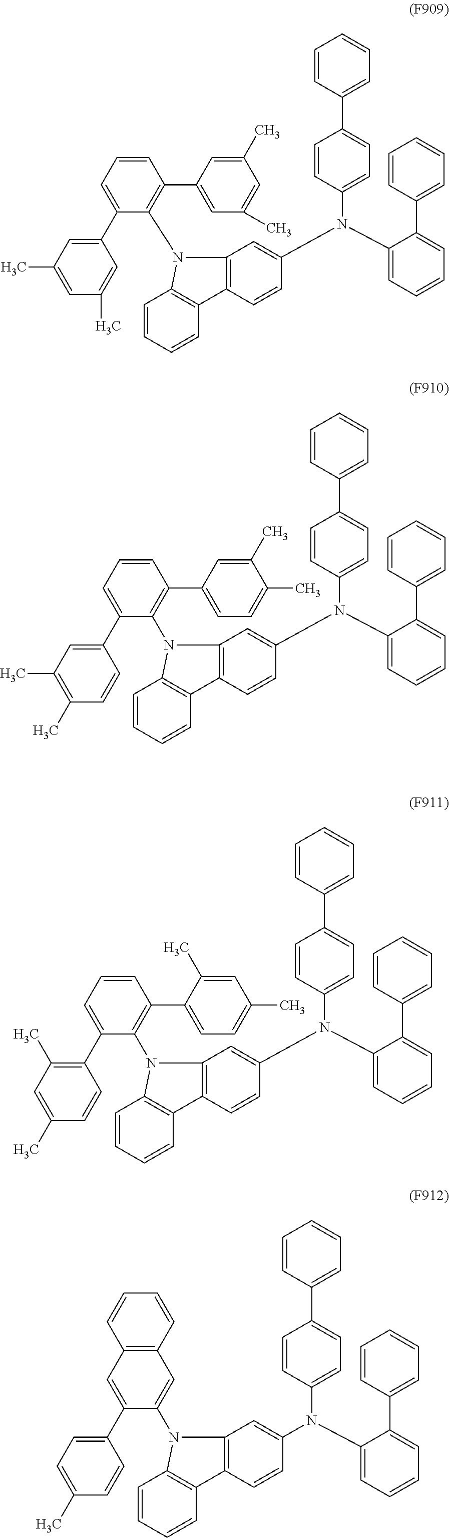 [SMILES: Cc1cc(C)cc(-c2cccc(-c3cc(C)cc(C)c3)c2-n2c3ccccc3c3ccc(N(c4ccc(-c5ccccc5)cc4)c4ccccc4-c4ccccc4)cc32)c1 ; Cc1ccc(-c2cccc(-c3ccc(C)c(C)c3)c2-n2c3ccccc3c3ccc(N(c4ccc(-c5ccccc5)cc4)c4ccccc4-c4ccccc4)cc32)cc1C ; Cc1ccc(-c2cccc(-c3ccc(C)cc3C)c2-n2c3ccccc3c3ccc(N(c4ccc(-c5ccccc5)cc4)c4ccccc4-c4ccccc4)cc32)c(C)c1 ; Cc1ccc(-c2cc3ccccc3cc2-n2c3ccccc3c3ccc(N(c4ccc(-c5ccccc5)cc4)c4ccccc4-c4ccccc4)cc32)cc1]

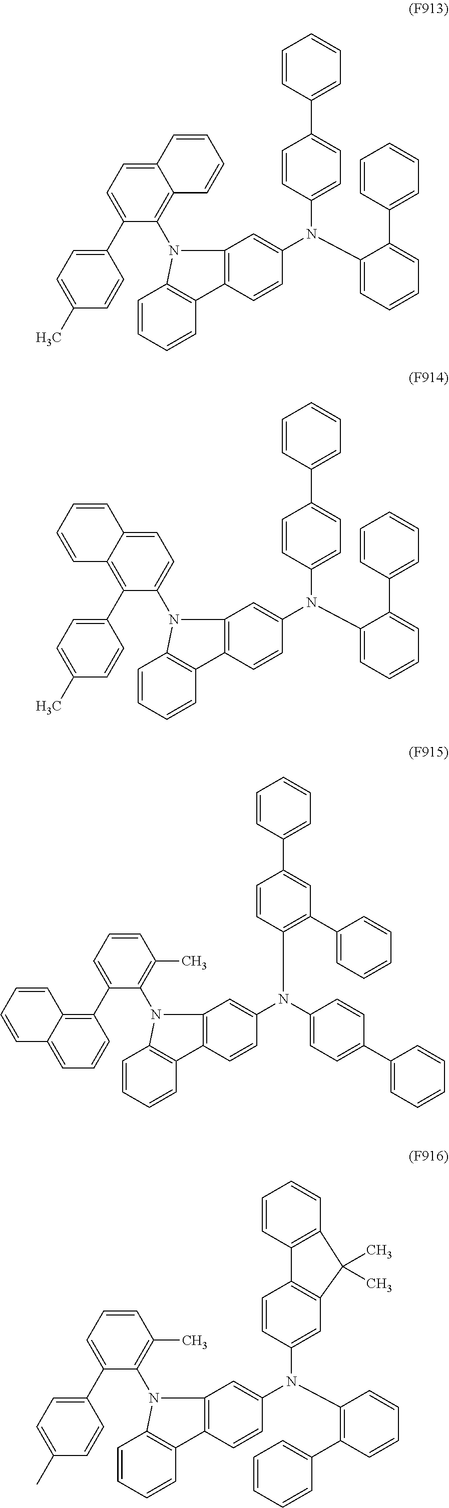 [SMILES: Cc1ccc(-c2ccc3ccccc3c2-n2c3ccccc3c3ccc(N(c4ccc(-c5ccccc5)cc4)c4ccccc4-c4ccccc4)cc32)cc1 ; Cc1ccc(-c2c(-n3c4ccccc4c4ccc(N(c5ccc(-c6ccccc6)cc5)c5ccccc5-c5ccccc5)cc43)ccc3ccccc23)cc1 ; Cc1cccc(-c2cccc3ccccc23)c1-n1c2ccccc2c2ccc(N(c3ccc(-c4ccccc4)cc3)c3ccc(-c4ccccc4)cc3-c3ccccc3)cc21 ; Cc1ccc(-c2cccc(C)c2-n2c3ccccc3c3ccc(N(c4ccc5c(c4)C(C)(C)c4ccccc4-5)c4ccccc4-c4ccccc4)cc32)cc1]

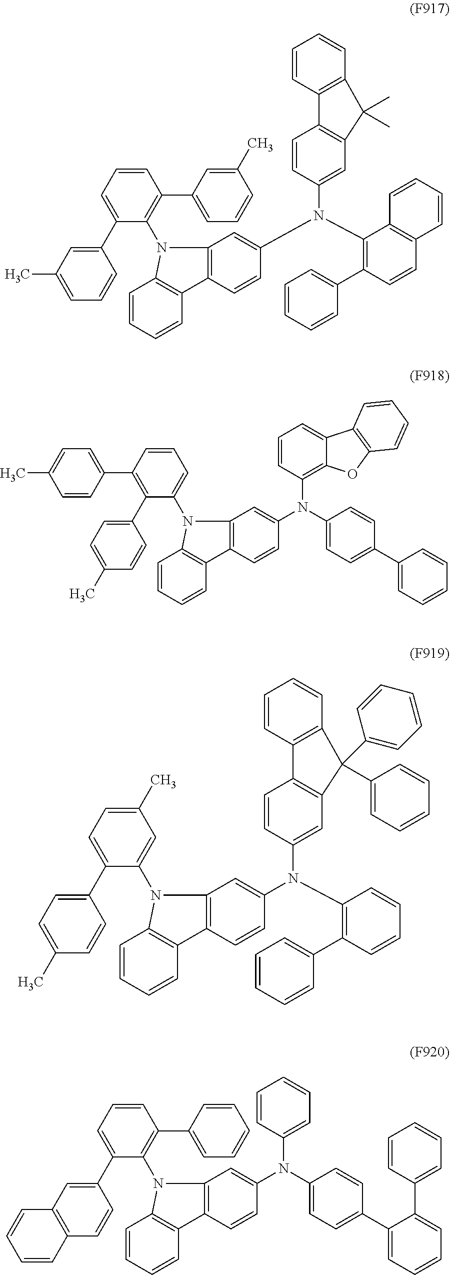 [SMILES: Cc1cccc(-c2cccc(-c3cccc(C)c3)c2-n2c3ccccc3c3ccc(N(c4ccc5c(c4)C(C)(C)c4ccccc4-5)c4c(-c5ccccc5)ccc5ccccc45)cc32)c1 ; Cc1ccc(-c2cccc(-n3c4ccccc4c4ccc(N(c5ccc(-c6ccccc6)cc5)c5cccc6c5oc5ccccc56)cc43)c2-c2ccc(C)cc2)cc1 ; Cc1ccc(-c2ccc(C)cc2-n2c3ccccc3c3ccc(N(c4ccc5c(c4)C(c4ccccc4)(c4ccccc4)c4ccccc4-5)c4ccccc4-c4ccccc4)cc32)cc1 ; c1ccc(-c2ccccc2-c2ccc(N(c3ccccc3)c3ccc4c5ccccc5n(-c5c(-c6ccccc6)cccc5-c5ccc6ccccc6c5)c4c3)cc2)cc1]

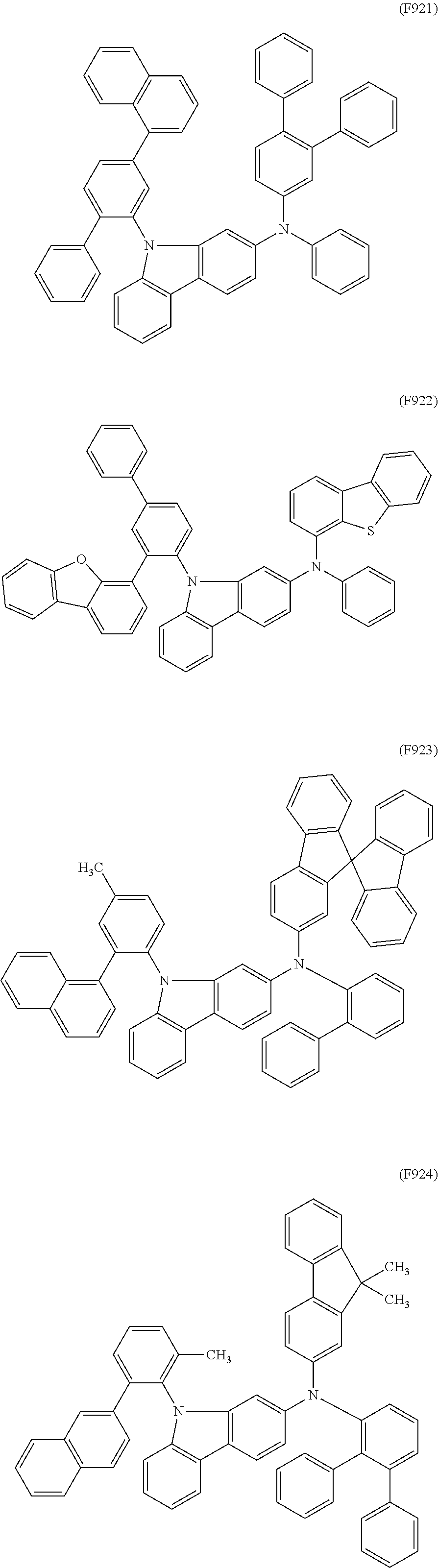 [SMILES: c1ccc(-c2ccc(N(c3ccccc3)c3ccc4c5ccccc5n(-c5cc(-c6cccc7ccccc67)ccc5-c5ccccc5)c4c3)cc2-c2ccccc2)cc1 ; c1ccc(-c2ccc(-n3c4ccccc4c4ccc(N(c5ccccc5)c5cccc6c5sc5ccccc56)cc43)c(-c3cccc4c3oc3ccccc34)c2)cc1 ; Cc1ccc(-n2c3ccccc3c3ccc(N(c4ccc5c(c4)C4(c6ccccc6-c6ccccc64)c4ccccc4-5)c4ccccc4-c4ccccc4)cc32)c(-c2cccc3ccccc23)c1 ; Cc1cccc(-c2ccc3ccccc3c2)c1-n1c2ccccc2c2ccc(N(c3ccc4c(c3)C(C)(C)c3ccccc3-4)c3cccc(-c4ccccc4)c3-c3ccccc3)cc21]

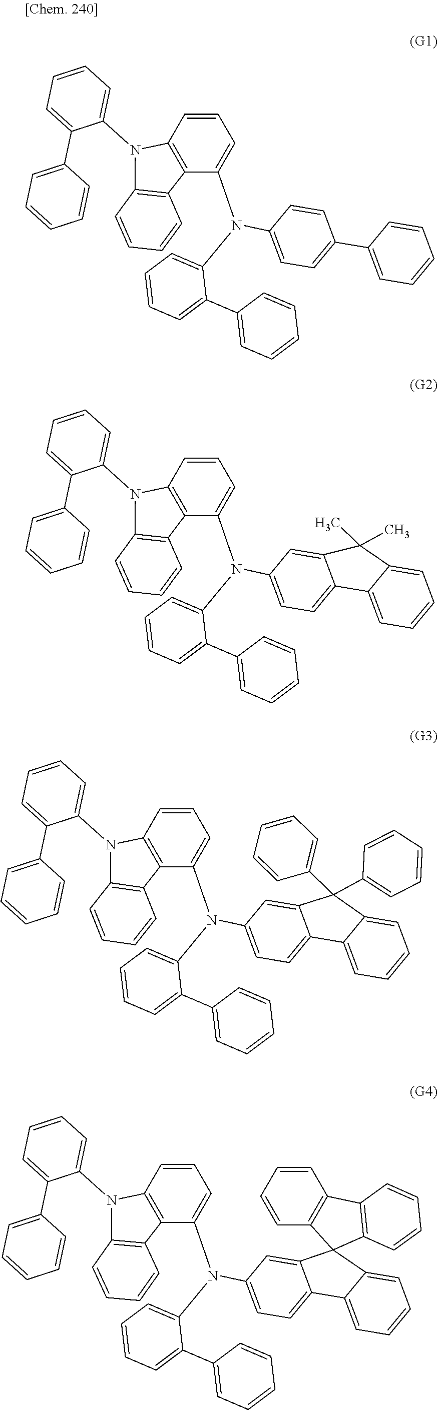 [SMILES: c1ccc(-c2ccc(N(c3ccccc3-c3ccccc3)c3cccc4c3c3ccccc3n4-c3ccccc3-c3ccccc3)cc2)cc1 ; CC1(C)c2ccccc2-c2ccc(N(c3ccccc3-c3ccccc3)c3cccc4c3c3ccccc3n4-c3ccccc3-c3ccccc3)cc21 ; c1ccc(-c2ccccc2N(c2ccc3c(c2)C(c2ccccc2)(c2ccccc2)c2ccccc2-3)c2cccc3c2c2ccccc2n3-c2ccccc2-c2ccccc2)cc1 ; c1ccc(-c2ccccc2N(c2ccc3c(c2)C2(c4ccccc4-c4ccccc42)c2ccccc2-3)c2cccc3c2c2ccccc2n3-c2ccccc2-c2ccccc2)cc1]

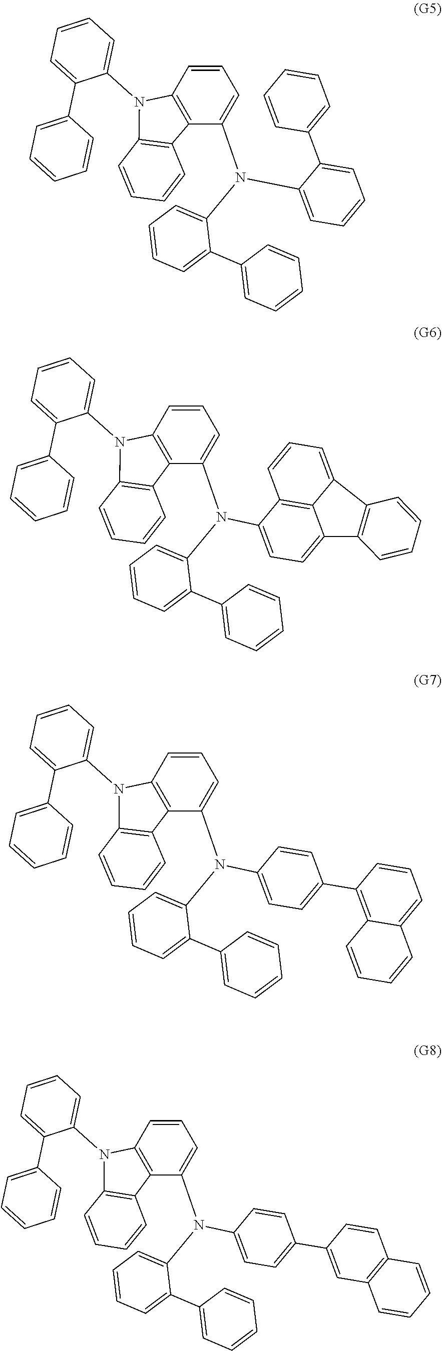 [SMILES: c1ccc(-c2ccccc2N(c2ccccc2-c2ccccc2)c2cccc3c2c2ccccc2n3-c2ccccc2-c2ccccc2)cc1 ; c1ccc(-c2ccccc2N(c2ccc3c4c(cccc24)-c2ccccc2-3)c2cccc3c2c2ccccc2n3-c2ccccc2-c2ccccc2)cc1 ; c1ccc(-c2ccccc2N(c2ccc(-c3cccc4ccccc34)cc2)c2cccc3c2c2ccccc2n3-c2ccccc2-c2ccccc2)cc1 ; c1ccc(-c2ccccc2N(c2ccc(-c3ccc4ccccc4c3)cc2)c2cccc3c2c2ccccc2n3-c2ccccc2-c2ccccc2)cc1]

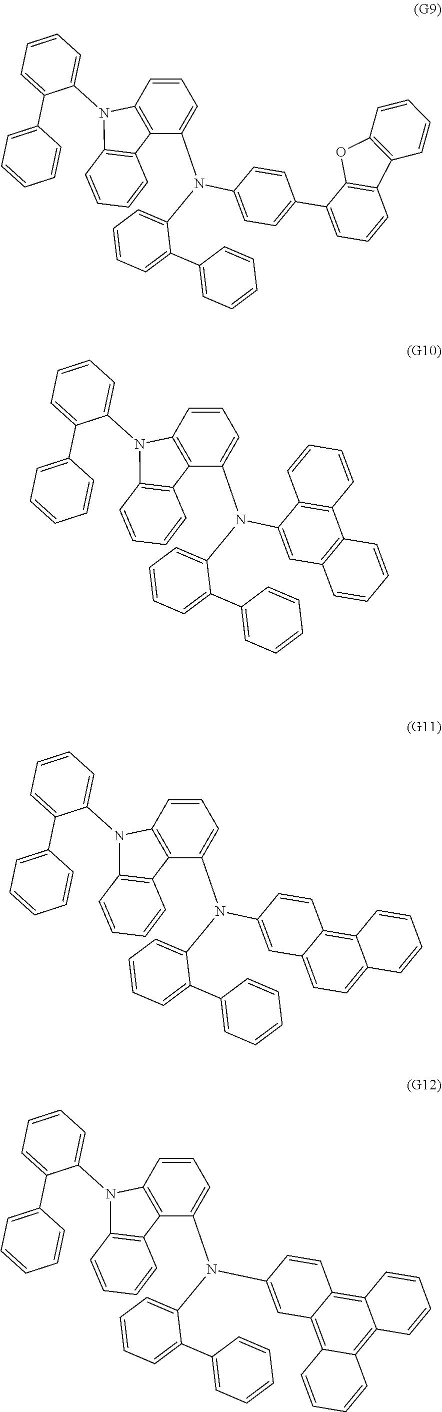 [SMILES: c1ccc(-c2ccccc2N(c2ccc(-c3cccc4c3oc3ccccc34)cc2)c2cccc3c2c2ccccc2n3-c2ccccc2-c2ccccc2)cc1 ; c1ccc(-c2ccccc2N(c2cc3ccccc3c3ccccc23)c2cccc3c2c2ccccc2n3-c2ccccc2-c2ccccc2)cc1 ; c1ccc(-c2ccccc2N(c2ccc3c(ccc4ccccc43)c2)c2cccc3c2c2ccccc2n3-c2ccccc2-c2ccccc2)cc1 ; c1ccc(-c2ccccc2N(c2ccc3c4ccccc4c4ccccc4c3c2)c2cccc3c2c2ccccc2n3-c2ccccc2-c2ccccc2)cc1]

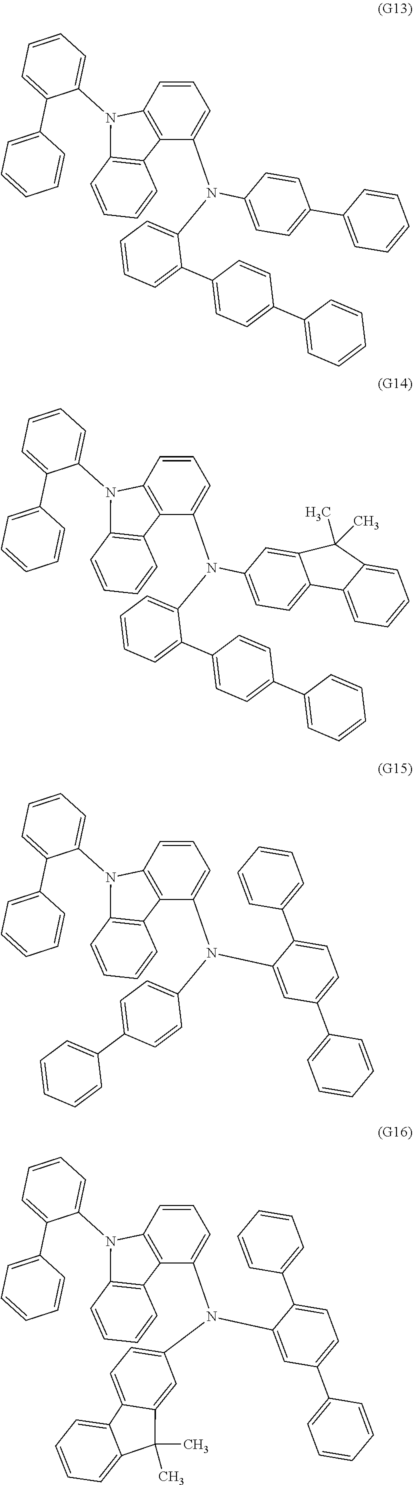 [SMILES: c1ccc(-c2ccc(-c3ccccc3N(c3ccc(-c4ccccc4)cc3)c3cccc4c3c3ccccc3n4-c3ccccc3-c3ccccc3)cc2)cc1 ; CC1(C)c2ccccc2-c2ccc(N(c3ccccc3-c3ccc(-c4ccccc4)cc3)c3cccc4c3c3ccccc3n4-c3ccccc3-c3ccccc3)cc21 ; c1ccc(-c2ccc(N(c3cc(-c4ccccc4)ccc3-c3ccccc3)c3cccc4c3c3ccccc3n4-c3ccccc3-c3ccccc3)cc2)cc1 ; CC1(C)c2ccccc2-c2ccc(N(c3cc(-c4ccccc4)ccc3-c3ccccc3)c3cccc4c3c3ccccc3n4-c3ccccc3-c3ccccc3)cc21]

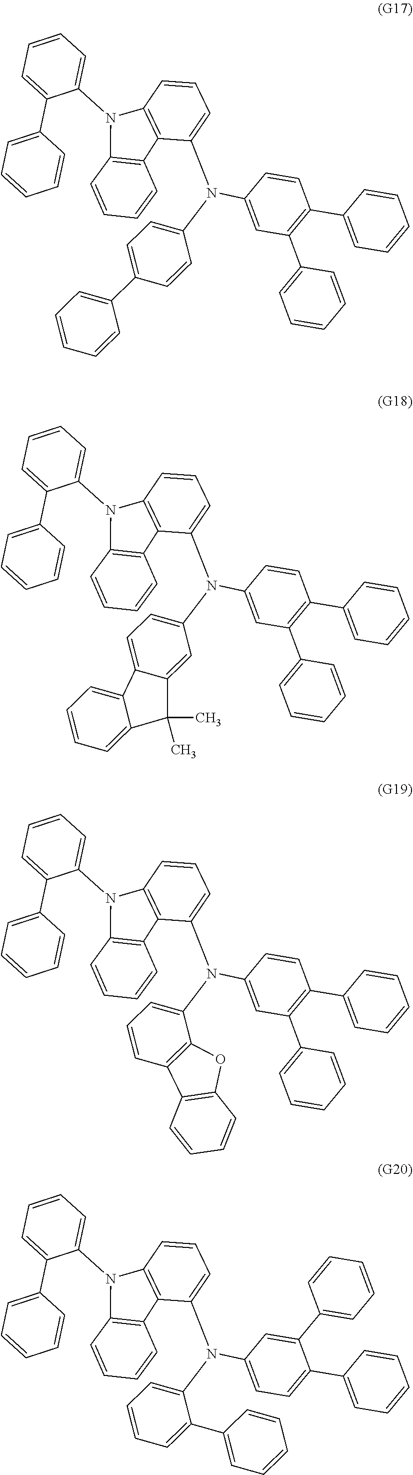 [SMILES: c1ccc(-c2ccc(N(c3ccc(-c4ccccc4)c(-c4ccccc4)c3)c3cccc4c3c3ccccc3n4-c3ccccc3-c3ccccc3)cc2)cc1 ; CC1(C)c2ccccc2-c2ccc(N(c3ccc(-c4ccccc4)c(-c4ccccc4)c3)c3cccc4c3c3ccccc3n4-c3ccccc3-c3ccccc3)cc21 ; c1ccc(-c2ccc(N(c3cccc4c3oc3ccccc34)c3cccc4c3c3ccccc3n4-c3ccccc3-c3ccccc3)cc2-c2ccccc2)cc1 ; c1ccc(-c2ccc(N(c3ccccc3-c3ccccc3)c3cccc4c3c3ccccc3n4-c3ccccc3-c3ccccc3)cc2-c2ccccc2)cc1]

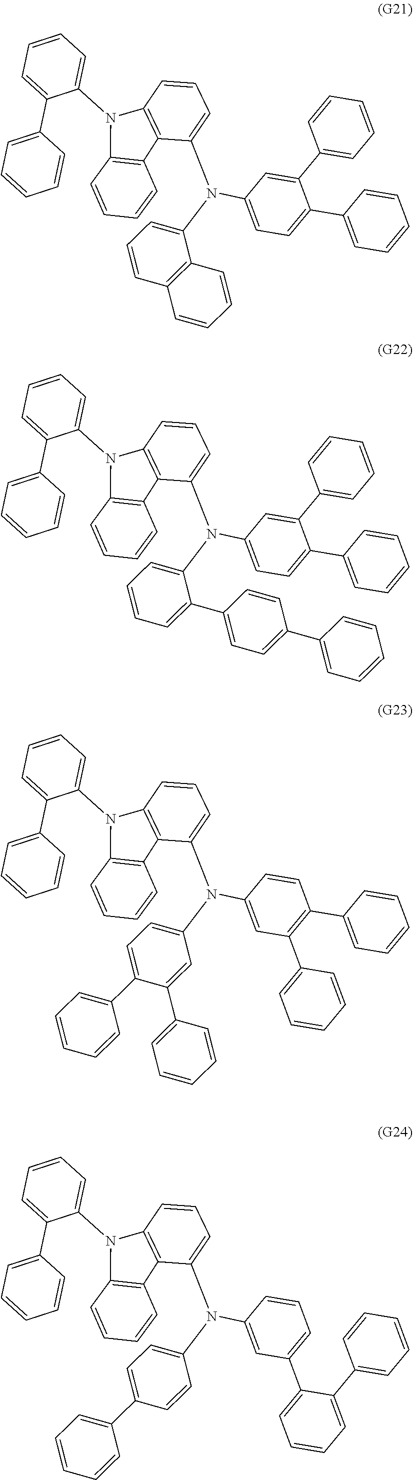 [SMILES: c1ccc(-c2ccc(N(c3cccc4ccccc34)c3cccc4c3c3ccccc3n4-c3ccccc3-c3ccccc3)cc2-c2ccccc2)cc1 ; c1ccc(-c2ccc(-c3ccccc3N(c3ccc(-c4ccccc4)c(-c4ccccc4)c3)c3cccc4c3c3ccccc3n4-c3ccccc3-c3ccccc3)cc2)cc1 ; c1ccc(-c2ccc(N(c3ccc(-c4ccccc4)c(-c4ccccc4)c3)c3cccc4c3c3ccccc3n4-c3ccccc3-c3ccccc3)cc2-c2ccccc2)cc1 ; c1ccc(-c2ccc(N(c3cccc(-c4ccccc4-c4ccccc4)c3)c3cccc4c3c3ccccc3n4-c3ccccc3-c3ccccc3)cc2)cc1]

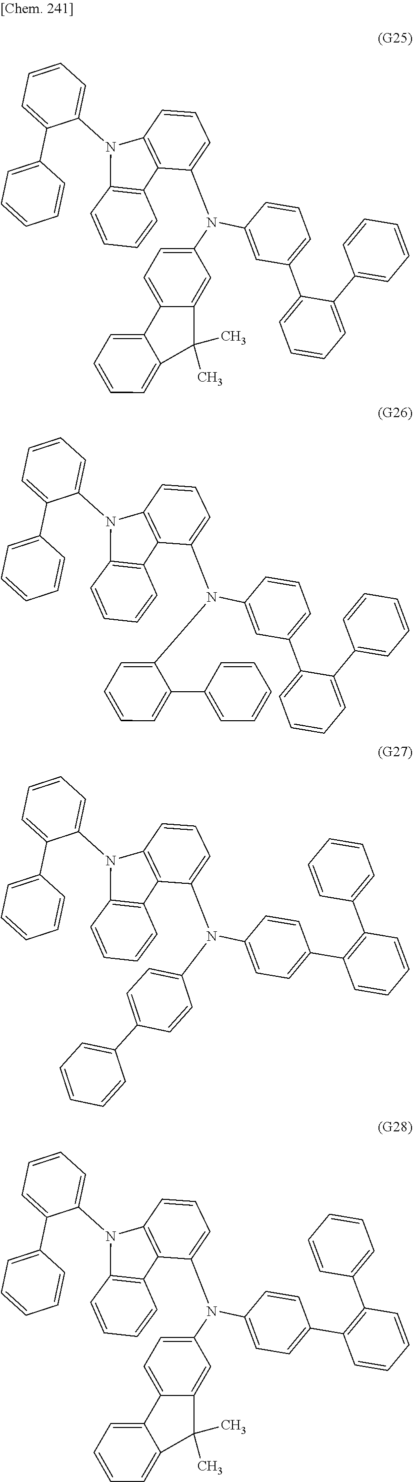 [SMILES: CC1(C)c2ccccc2-c2ccc(N(c3cccc(-c4ccccc4-c4ccccc4)c3)c3cccc4c3c3ccccc3n4-c3ccccc3-c3ccccc3)cc21 ; c1ccc(-c2ccccc2-c2cccc(N(c3ccccc3-c3ccccc3)c3cccc4c3c3ccccc3n4-c3ccccc3-c3ccccc3)c2)cc1 ; c1ccc(-c2ccc(N(c3ccc(-c4ccccc4-c4ccccc4)cc3)c3cccc4c3c3ccccc3n4-c3ccccc3-c3ccccc3)cc2)cc1 ; CC1(C)c2ccccc2-c2ccc(N(c3ccc(-c4ccccc4-c4ccccc4)cc3)c3cccc4c3c3ccccc3n4-c3ccccc3-c3ccccc3)cc21]

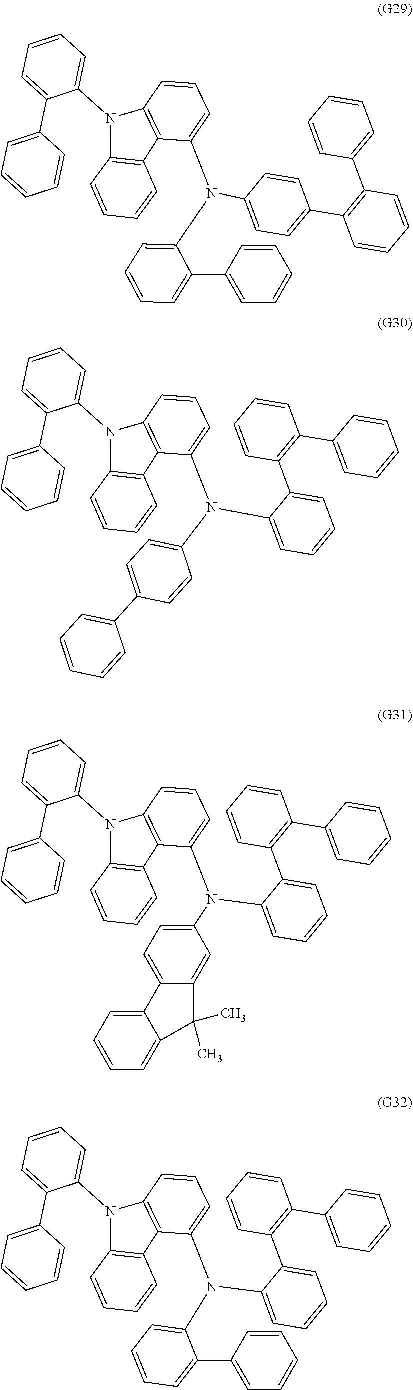 [SMILES: c1ccc(-c2ccccc2-c2ccc(N(c3ccccc3-c3ccccc3)c3cccc4c3c3ccccc3n4-c3ccccc3-c3ccccc3)cc2)cc1 ; c1ccc(-c2ccc(N(c3ccccc3-c3ccccc3-c3ccccc3)c3cccc4c3c3ccccc3n4-c3ccccc3-c3ccccc3)cc2)cc1 ; CC1(C)c2ccccc2-c2ccc(N(c3ccccc3-c3ccccc3-c3ccccc3)c3cccc4c3c3ccccc3n4-c3ccccc3-c3ccccc3)cc21 ; c1ccc(-c2ccccc2-c2ccccc2N(c2ccccc2-c2ccccc2)c2cccc3c2c2ccccc2n3-c2ccccc2-c2ccccc2)cc1]

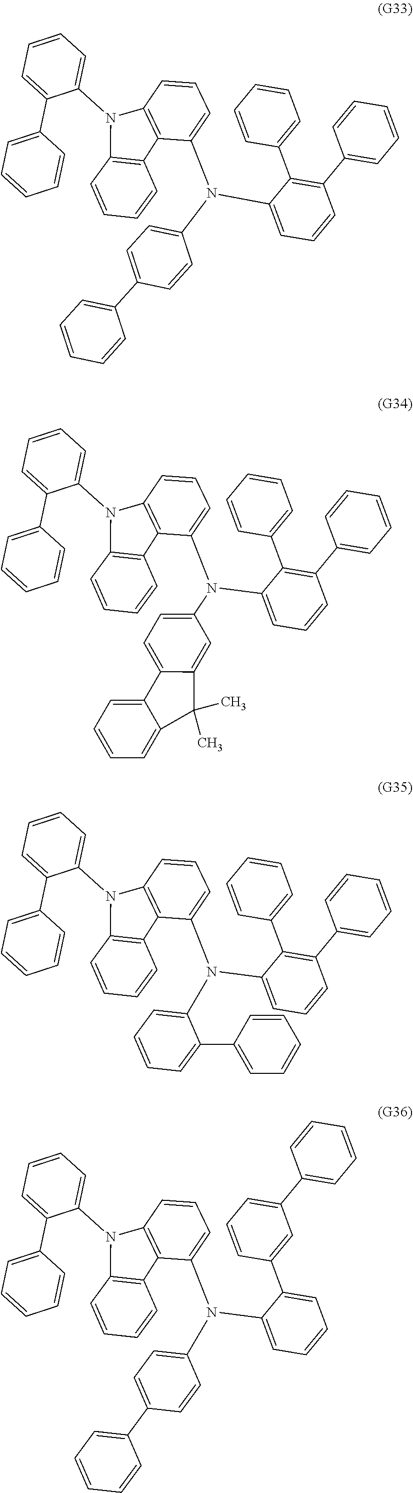 [SMILES: c1ccc(-c2ccc(N(c3cccc(-c4ccccc4)c3-c3ccccc3)c3cccc4c3c3ccccc3n4-c3ccccc3-c3ccccc3)cc2)cc1 ; CC1(C)c2ccccc2-c2ccc(N(c3cccc(-c4ccccc4)c3-c3ccccc3)c3cccc4c3c3ccccc3n4-c3ccccc3-c3ccccc3)cc21 ; c1ccc(-c2ccccc2N(c2cccc(-c3ccccc3)c2-c2ccccc2)c2cccc3c2c2ccccc2n3-c2ccccc2-c2ccccc2)cc1 ; c1ccc(-c2ccc(N(c3ccccc3-c3cccc(-c4ccccc4)c3)c3cccc4c3c3ccccc3n4-c3ccccc3-c3ccccc3)cc2)cc1]

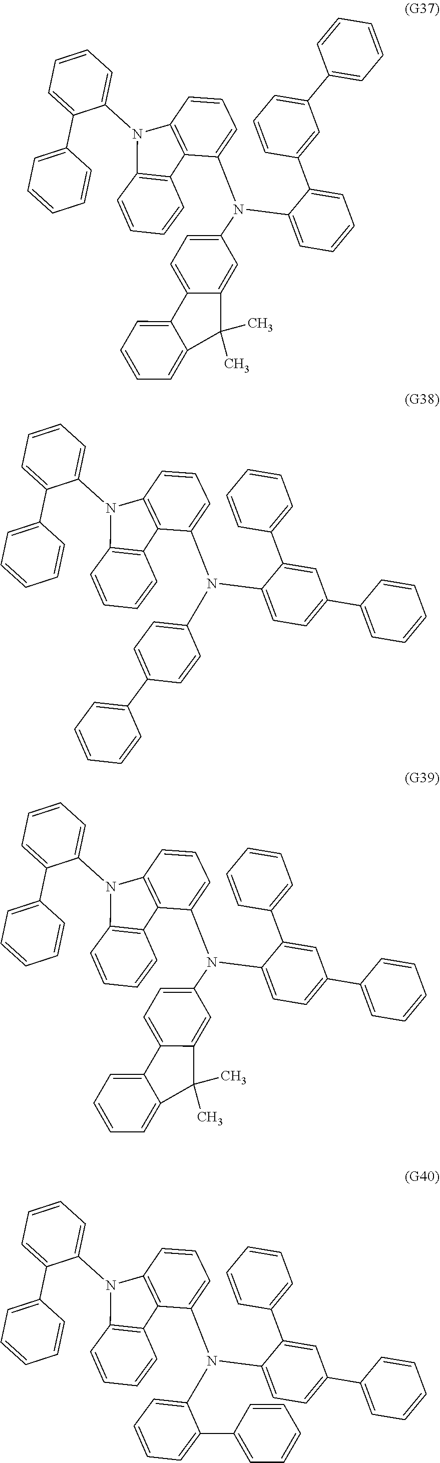 [SMILES: CC1(C)c2ccccc2-c2ccc(N(c3ccccc3-c3cccc(-c4ccccc4)c3)c3cccc4c3c3ccccc3n4-c3ccccc3-c3ccccc3)cc21 ; c1ccc(-c2ccc(N(c3ccc(-c4ccccc4)cc3-c3ccccc3)c3cccc4c3c3ccccc3n4-c3ccccc3-c3ccccc3)cc2)cc1 ; CC1(C)c2ccccc2-c2ccc(N(c3ccc(-c4ccccc4)cc3-c3ccccc3)c3cccc4c3c3ccccc3n4-c3ccccc3-c3ccccc3)cc21 ; c1ccc(-c2ccc(N(c3ccccc3-c3ccccc3)c3cccc4c3c3ccccc3n4-c3ccccc3-c3ccccc3)c(-c3ccccc3)c2)cc1]

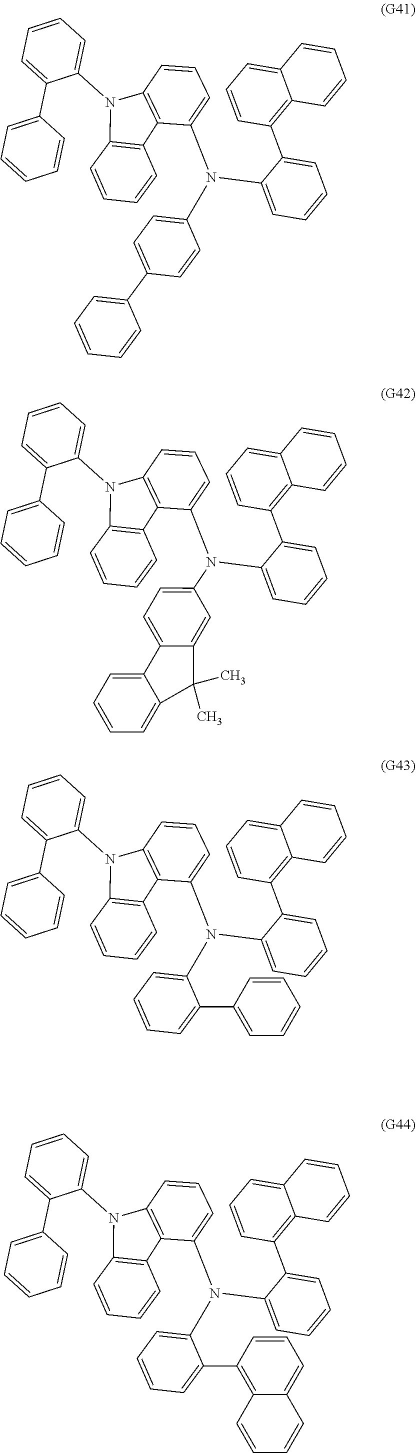 [SMILES: c1ccc(-c2ccc(N(c3ccccc3-c3cccc4ccccc34)c3cccc4c3c3ccccc3n4-c3ccccc3-c3ccccc3)cc2)cc1 ; CC1(C)c2ccccc2-c2ccc(N(c3ccccc3-c3cccc4ccccc34)c3cccc4c3c3ccccc3n4-c3ccccc3-c3ccccc3)cc21 ; c1ccc(-c2ccccc2N(c2ccccc2-c2cccc3ccccc23)c2cccc3c2c2ccccc2n3-c2ccccc2-c2ccccc2)cc1 ; c1ccc(-c2ccccc2-n2c3ccccc3c3c(N(c4ccccc4-c4cccc5ccccc45)c4ccccc4-c4cccc5ccccc45)cccc32)cc1]

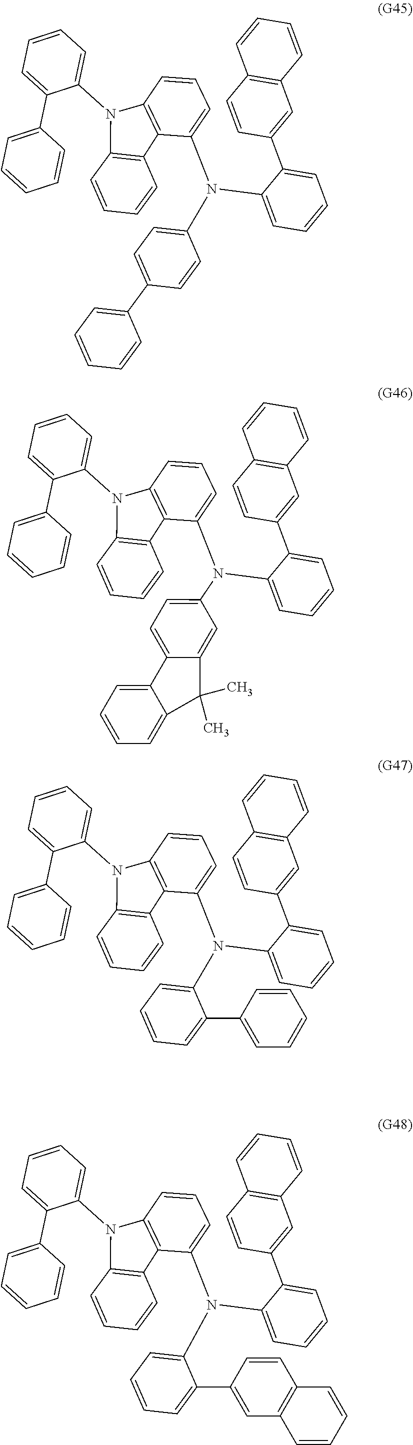 [SMILES: c1ccc(-c2ccc(N(c3ccccc3-c3ccc4ccccc4c3)c3cccc4c3c3ccccc3n4-c3ccccc3-c3ccccc3)cc2)cc1 ; CC1(C)c2ccccc2-c2ccc(N(c3ccccc3-c3ccc4ccccc4c3)c3cccc4c3c3ccccc3n4-c3ccccc3-c3ccccc3)cc21 ; c1ccc(-c2ccccc2N(c2ccccc2-c2ccc3ccccc3c2)c2cccc3c2c2ccccc2n3-c2ccccc2-c2ccccc2)cc1 ; c1ccc(-c2ccccc2-n2c3ccccc3c3c(N(c4ccccc4-c4ccc5ccccc5c4)c4ccccc4-c4ccc5ccccc5c4)cccc32)cc1]

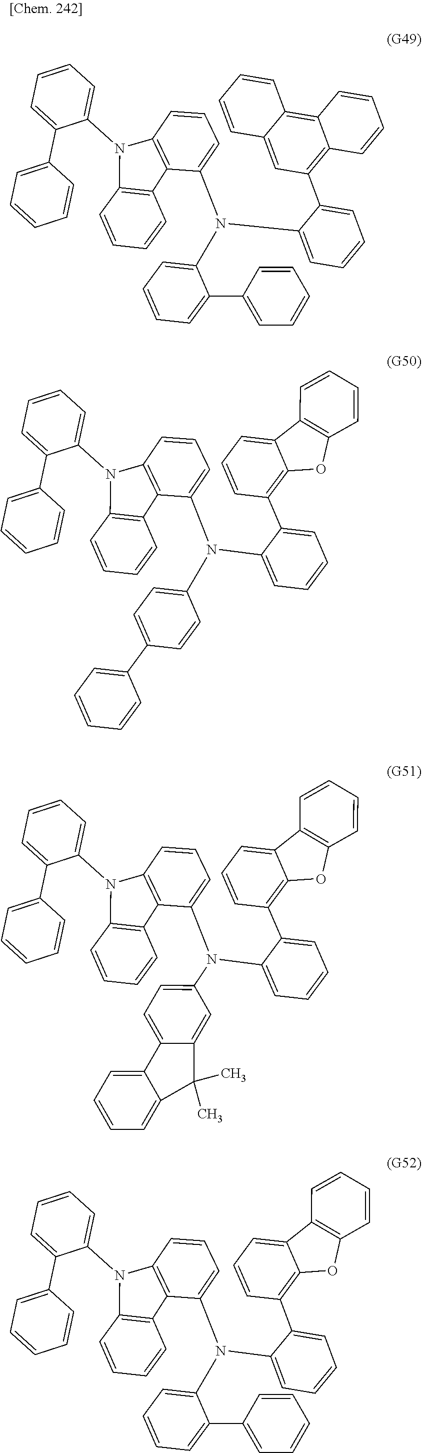 [SMILES: c1ccc(-c2ccccc2N(c2ccccc2-c2cc3ccccc3c3ccccc23)c2cccc3c2c2ccccc2n3-c2ccccc2-c2ccccc2)cc1 ; c1ccc(-c2ccc(N(c3ccccc3-c3cccc4c3oc3ccccc34)c3cccc4c3c3ccccc3n4-c3ccccc3-c3ccccc3)cc2)cc1 ; CC1(C)c2ccccc2-c2ccc(N(c3ccccc3-c3cccc4c3oc3ccccc34)c3cccc4c3c3ccccc3n4-c3ccccc3-c3ccccc3)cc21 ; c1ccc(-c2ccccc2N(c2ccccc2-c2cccc3c2oc2ccccc23)c2cccc3c2c2ccccc2n3-c2ccccc2-c2ccccc2)cc1]

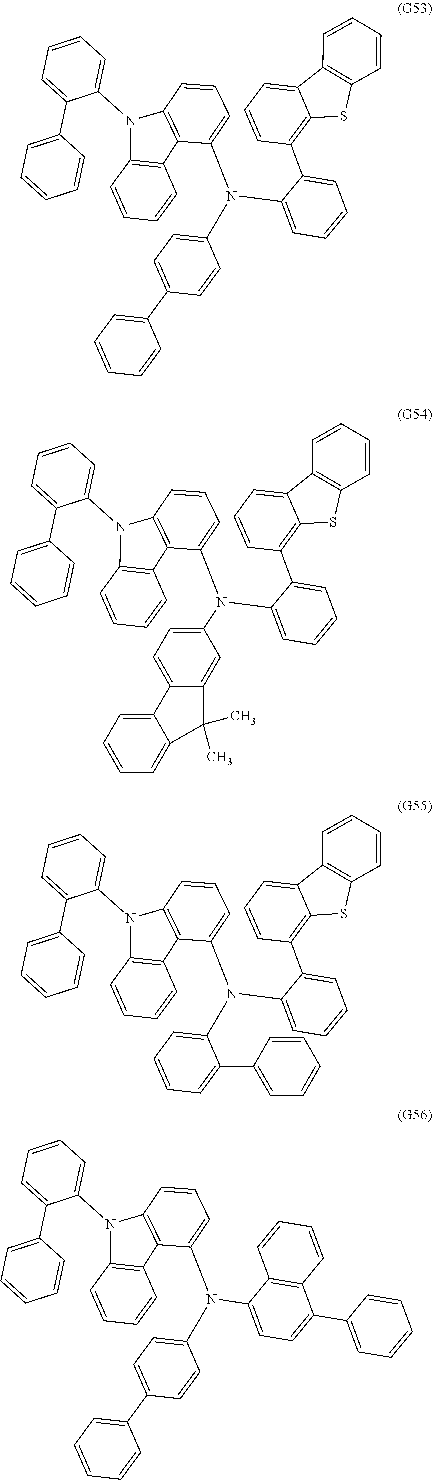 [SMILES: c1ccc(-c2ccc(N(c3ccccc3-c3cccc4c3sc3ccccc34)c3cccc4c3c3ccccc3n4-c3ccccc3-c3ccccc3)cc2)cc1 ; CC1(C)c2ccccc2-c2ccc(N(c3ccccc3-c3cccc4c3sc3ccccc34)c3cccc4c3c3ccccc3n4-c3ccccc3-c3ccccc3)cc21 ; c1ccc(-c2ccccc2N(c2ccccc2-c2cccc3c2sc2ccccc23)c2cccc3c2c2ccccc2n3-c2ccccc2-c2ccccc2)cc1 ; c1ccc(-c2ccc(N(c3ccc(-c4ccccc4)c4ccccc34)c3cccc4c3c3ccccc3n4-c3ccccc3-c3ccccc3)cc2)cc1]

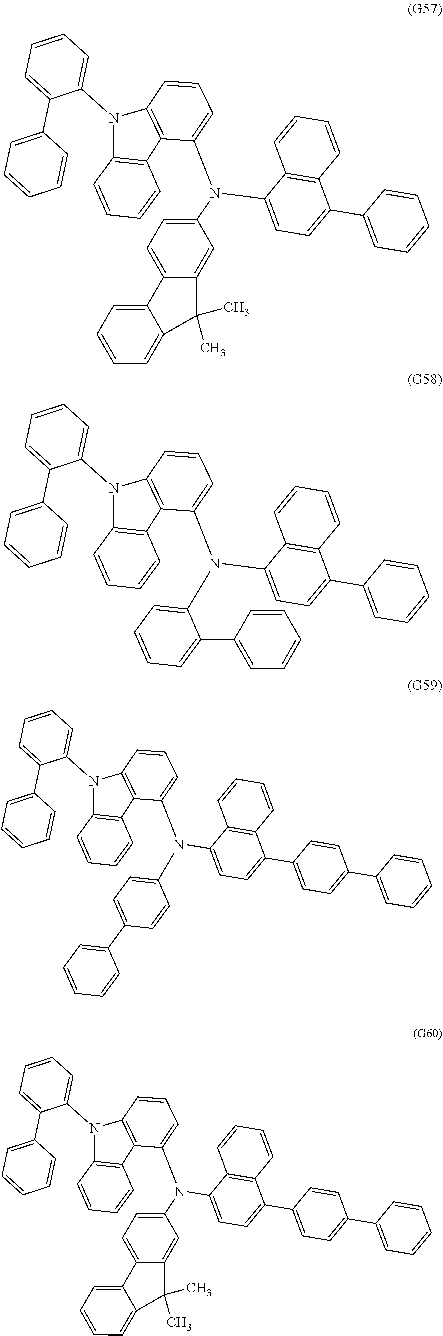 [SMILES: CC1(C)c2ccccc2-c2ccc(N(c3ccc(-c4ccccc4)c4ccccc34)c3cccc4c3c3ccccc3n4-c3ccccc3-c3ccccc3)cc21 ; c1ccc(-c2ccccc2N(c2ccc(-c3ccccc3)c3ccccc23)c2cccc3c2c2ccccc2n3-c2ccccc2-c2ccccc2)cc1 ; c1ccc(-c2ccc(-c3ccc(N(c4ccc(-c5ccccc5)cc4)c4cccc5c4c4ccccc4n5-c4ccccc4-c4ccccc4)c4ccccc34)cc2)cc1 ; CC1(C)c2ccccc2-c2ccc(N(c3ccc(-c4ccc(-c5ccccc5)cc4)c4ccccc34)c3cccc4c3c3ccccc3n4-c3ccccc3-c3ccccc3)cc21]

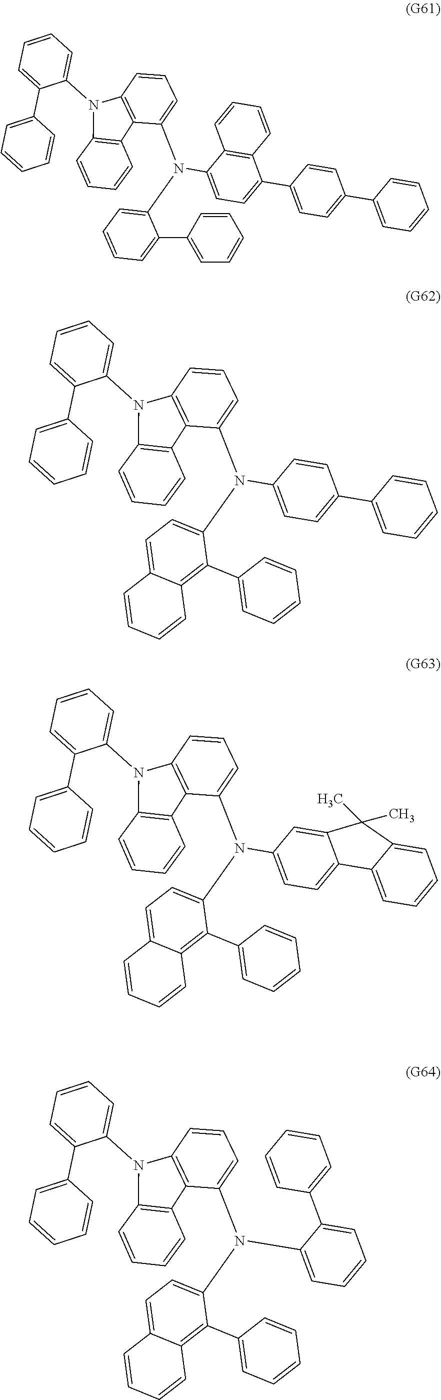 [SMILES: c1ccc(-c2ccc(-c3ccc(N(c4ccccc4-c4ccccc4)c4cccc5c4c4ccccc4n5-c4ccccc4-c4ccccc4)c4ccccc34)cc2)cc1 ; c1ccc(-c2ccc(N(c3ccc4ccccc4c3-c3ccccc3)c3cccc4c3c3ccccc3n4-c3ccccc3-c3ccccc3)cc2)cc1 ; CC1(C)c2ccccc2-c2ccc(N(c3ccc4ccccc4c3-c3ccccc3)c3cccc4c3c3ccccc3n4-c3ccccc3-c3ccccc3)cc21 ; c1ccc(-c2ccccc2N(c2ccc3ccccc3c2-c2ccccc2)c2cccc3c2c2ccccc2n3-c2ccccc2-c2ccccc2)cc1]

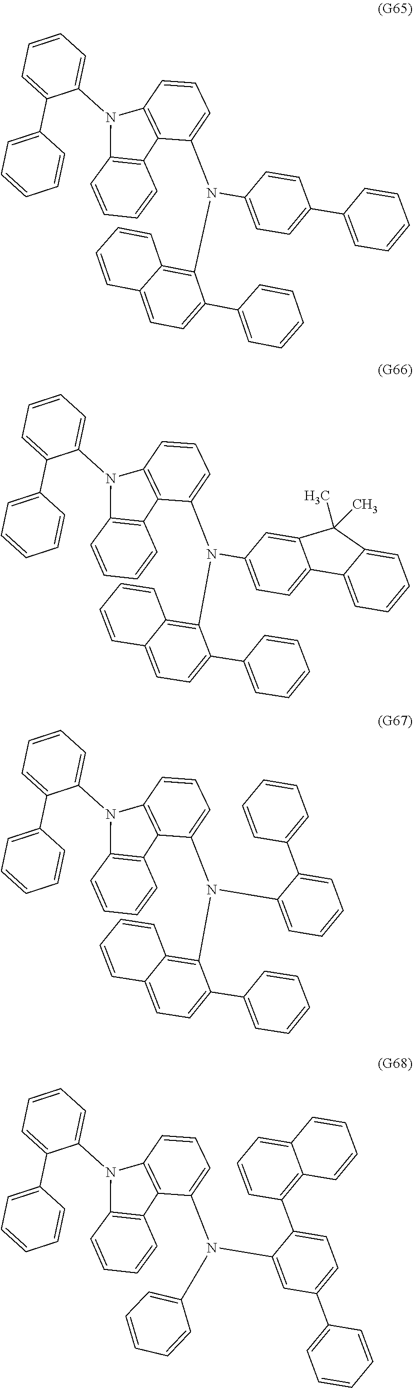 [SMILES: c1ccc(-c2ccc(N(c3c(-c4ccccc4)ccc4ccccc34)c3cccc4c3c3ccccc3n4-c3ccccc3-c3ccccc3)cc2)cc1 ; CC1(C)c2ccccc2-c2ccc(N(c3c(-c4ccccc4)ccc4ccccc34)c3cccc4c3c3ccccc3n4-c3ccccc3-c3ccccc3)cc21 ; c1ccc(-c2ccccc2N(c2c(-c3ccccc3)ccc3ccccc23)c2cccc3c2c2ccccc2n3-c2ccccc2-c2ccccc2)cc1 ; c1ccc(-c2ccc(-c3cccc4ccccc34)c(N(c3ccccc3)c3cccc4c3c3ccccc3n4-c3ccccc3-c3ccccc3)c2)cc1]

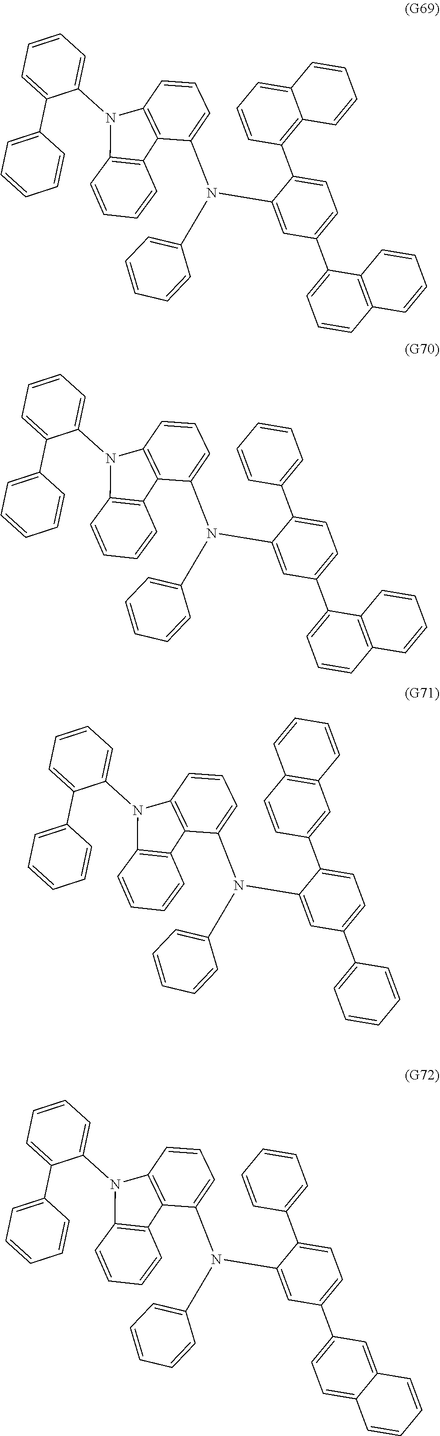 [SMILES: c1ccc(-c2ccccc2-n2c3ccccc3c3c(N(c4ccccc4)c4cc(-c5cccc6ccccc56)ccc4-c4cccc5ccccc45)cccc32)cc1 ; c1ccc(-c2ccc(-c3cccc4ccccc34)cc2N(c2ccccc2)c2cccc3c2c2ccccc2n3-c2ccccc2-c2ccccc2)cc1 ; c1ccc(-c2ccc(-c3ccc4ccccc4c3)c(N(c3ccccc3)c3cccc4c3c3ccccc3n4-c3ccccc3-c3ccccc3)c2)cc1 ; c1ccc(-c2ccc(-c3ccc4ccccc4c3)cc2N(c2ccccc2)c2cccc3c2c2ccccc2n3-c2ccccc2-c2ccccc2)cc1]

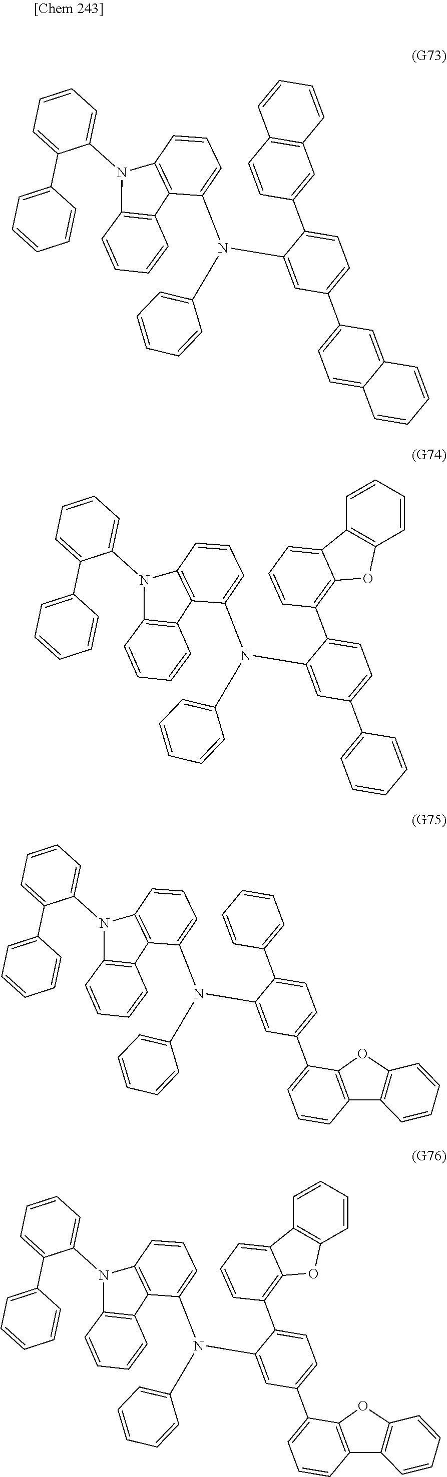 [SMILES: c1ccc(-c2ccccc2-n2c3ccccc3c3c(N(c4ccccc4)c4cc(-c5ccc6ccccc6c5)ccc4-c4ccc5ccccc5c4)cccc32)cc1 ; c1ccc(-c2ccc(-c3cccc4c3oc3ccccc34)c(N(c3ccccc3)c3cccc4c3c3ccccc3n4-c3ccccc3-c3ccccc3)c2)cc1 ; c1ccc(-c2ccc(-c3cccc4c3oc3ccccc34)cc2N(c2ccccc2)c2cccc3c2c2ccccc2n3-c2ccccc2-c2ccccc2)cc1 ; c1ccc(-c2ccccc2-n2c3ccccc3c3c(N(c4ccccc4)c4cc(-c5cccc6c5oc5ccccc56)ccc4-c4cccc5c4oc4ccccc45)cccc32)cc1]

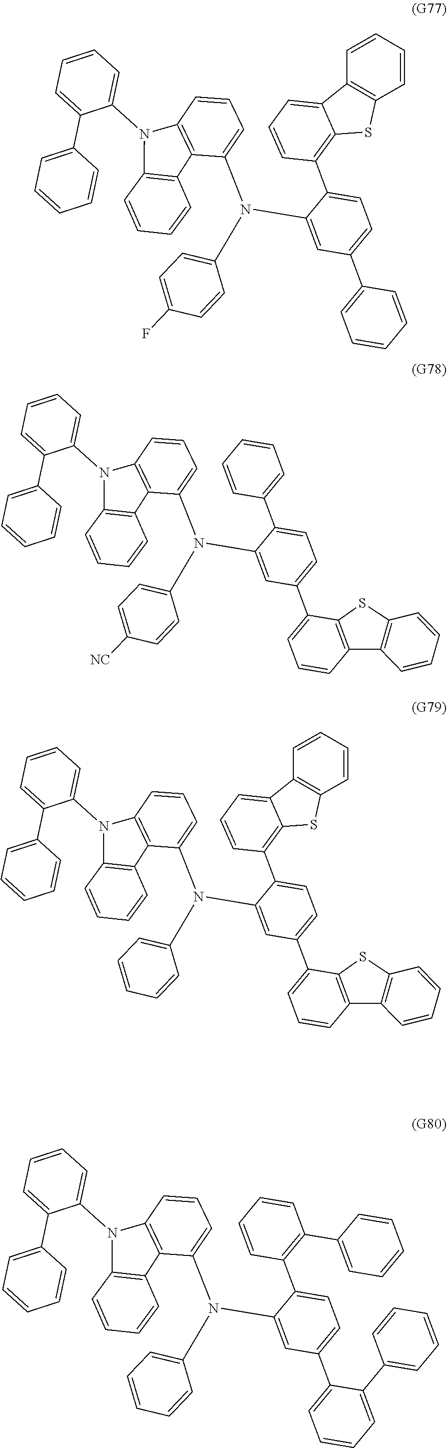 [SMILES: Fc1ccc(N(c2cc(-c3ccccc3)ccc2-c2cccc3c2sc2ccccc23)c2cccc3c2c2ccccc2n3-c2ccccc2-c2ccccc2)cc1 ; N#Cc1ccc(N(c2cc(-c3cccc4c3sc3ccccc34)ccc2-c2ccccc2)c2cccc3c2c2ccccc2n3-c2ccccc2-c2ccccc2)cc1 ; c1ccc(-c2ccccc2-n2c3ccccc3c3c(N(c4ccccc4)c4cc(-c5cccc6c5sc5ccccc56)ccc4-c4cccc5c4sc4ccccc45)cccc32)cc1 ; c1ccc(-c2ccccc2-c2ccc(-c3ccccc3-c3ccccc3)c(N(c3ccccc3)c3cccc4c3c3ccccc3n4-c3ccccc3-c3ccccc3)c2)cc1]

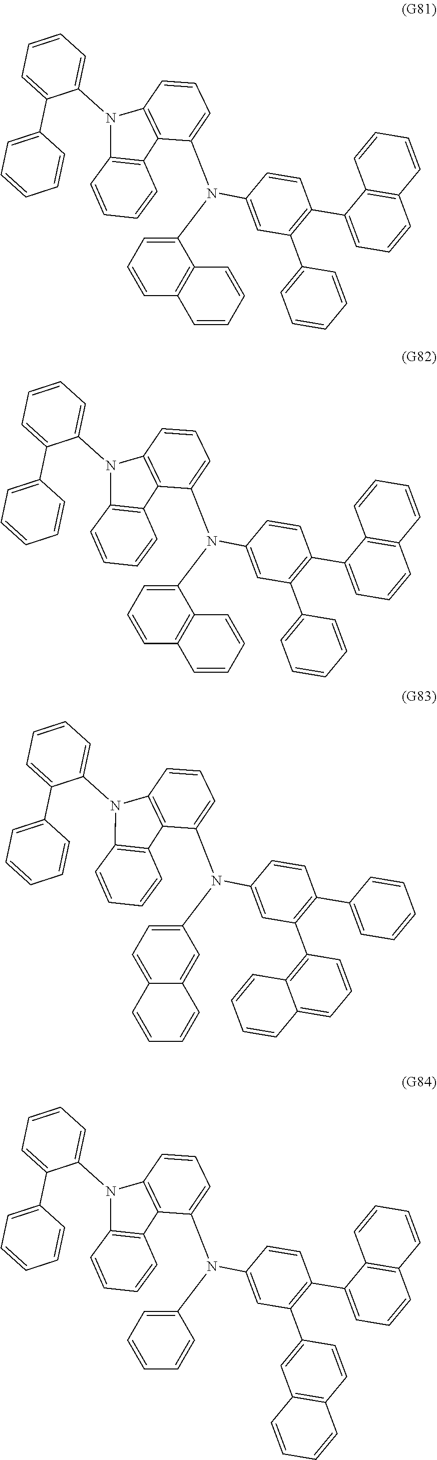 [SMILES: c1ccc(-c2cc(N(c3cccc4ccccc34)c3cccc4c3c3ccccc3n4-c3ccccc3-c3ccccc3)ccc2-c2cccc3ccccc23)cc1 ; c1ccc(-c2cc(N(c3cccc4ccccc34)c3cccc4c3c3ccccc3n4-c3ccccc3-c3ccccc3)ccc2-c2cccc3ccccc23)cc1 ; c1ccc(-c2ccc(N(c3ccc4ccccc4c3)c3cccc4c3c3ccccc3n4-c3ccccc3-c3ccccc3)cc2-c2cccc3ccccc23)cc1 ; c1ccc(-c2ccccc2-n2c3ccccc3c3c(N(c4ccccc4)c4ccc(-c5cccc6ccccc56)c(-c5ccc6ccccc6c5)c4)cccc32)cc1]

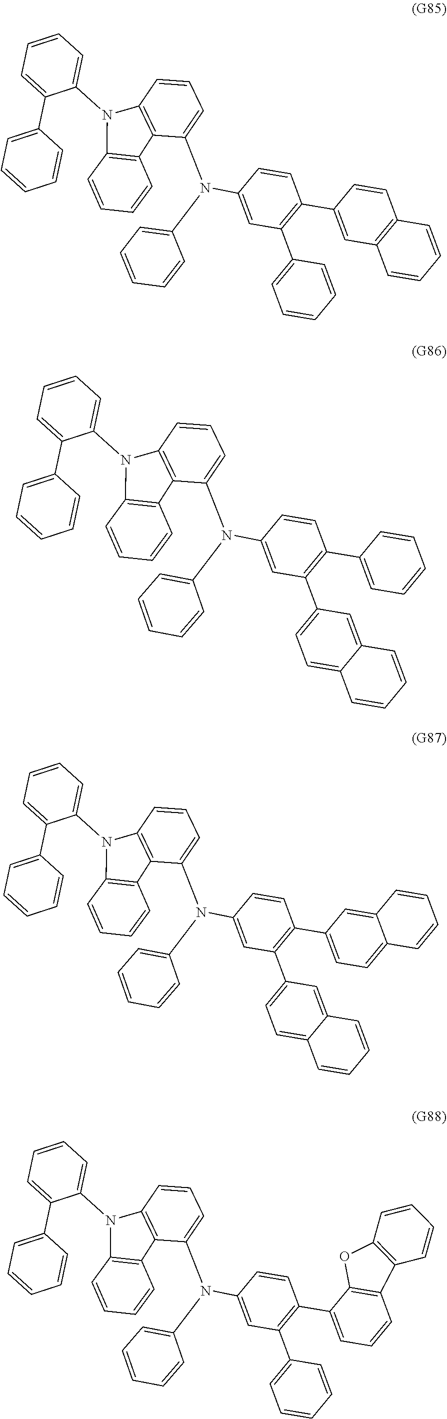 [SMILES: c1ccc(-c2cc(N(c3ccccc3)c3cccc4c3c3ccccc3n4-c3ccccc3-c3ccccc3)ccc2-c2ccc3ccccc3c2)cc1 ; c1ccc(-c2ccc(N(c3ccccc3)c3cccc4c3c3ccccc3n4-c3ccccc3-c3ccccc3)cc2-c2ccc3ccccc3c2)cc1 ; c1ccc(-c2ccccc2-n2c3ccccc3c3c(N(c4ccccc4)c4ccc(-c5ccc6ccccc6c5)c(-c5ccc6ccccc6c5)c4)cccc32)cc1 ; c1ccc(-c2cc(N(c3ccccc3)c3cccc4c3c3ccccc3n4-c3ccccc3-c3ccccc3)ccc2-c2cccc3c2oc2ccccc23)cc1]

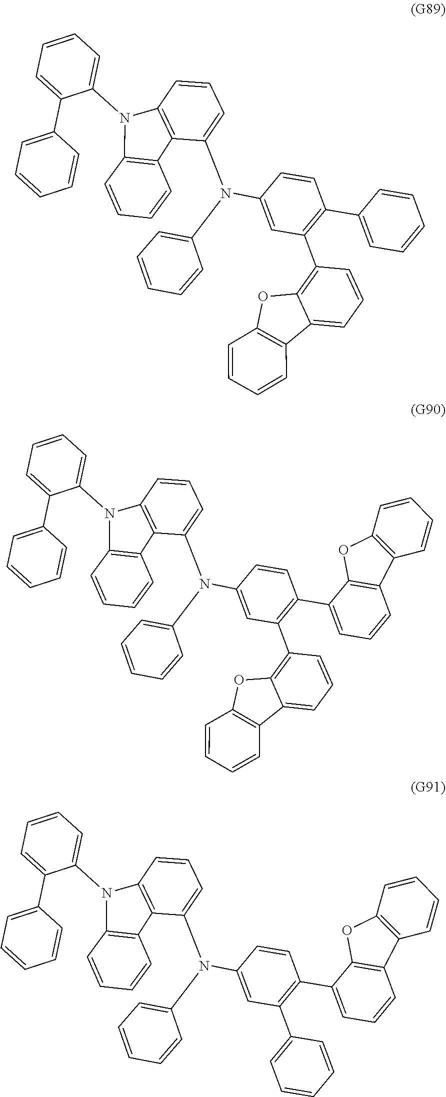 [SMILES: c1ccc(-c2ccc(N(c3ccccc3)c3cccc4c3c3ccccc3n4-c3ccccc3-c3ccccc3)cc2-c2cccc3c2oc2ccccc23)cc1 ; c1ccc(-c2ccccc2-n2c3ccccc3c3c(N(c4ccccc4)c4ccc(-c5cccc6c5oc5ccccc56)c(-c5cccc6c5oc5ccccc56)c4)cccc32)cc1 ; c1ccc(-c2cc(N(c3ccccc3)c3cccc4c3c3ccccc3n4-c3ccccc3-c3ccccc3)ccc2-c2cccc3c2oc2ccccc23)cc1]

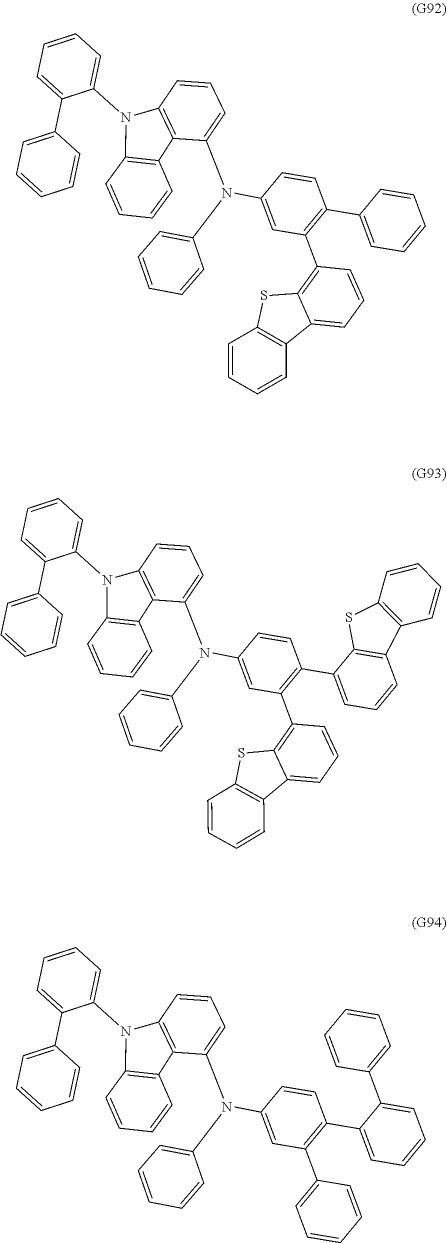 [SMILES: c1ccc(-c2ccc(N(c3ccccc3)c3cccc4c3c3ccccc3n4-c3ccccc3-c3ccccc3)cc2-c2cccc3c2sc2ccccc23)cc1 ; c1ccc(-c2ccccc2-n2c3ccccc3c3c(N(c4ccccc4)c4ccc(-c5cccc6c5sc5ccccc56)c(-c5cccc6c5sc5ccccc56)c4)cccc32)cc1 ; c1ccc(-c2ccccc2-c2ccc(N(c3ccccc3)c3cccc4c3c3ccccc3n4-c3ccccc3-c3ccccc3)cc2-c2ccccc2)cc1]

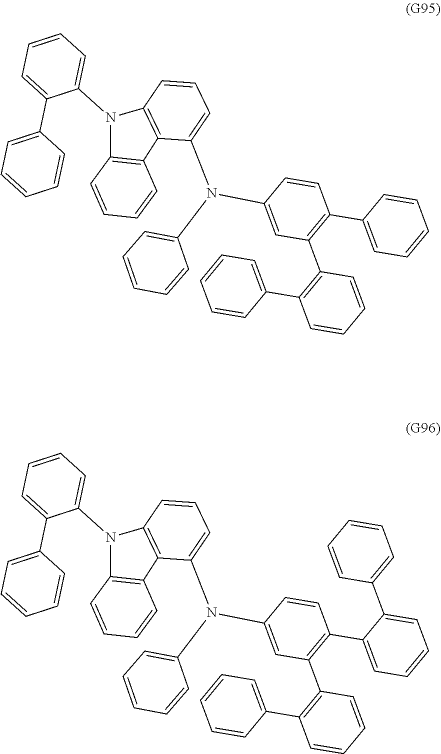 [SMILES: c1ccc(-c2ccccc2-c2cc(N(c3ccccc3)c3cccc4c3c3ccccc3n4-c3ccccc3-c3ccccc3)ccc2-c2ccccc2)cc1 ; c1ccc(-c2ccccc2-c2ccc(N(c3ccccc3)c3cccc4c3c3ccccc3n4-c3ccccc3-c3ccccc3)cc2-c2ccccc2-c2ccccc2)cc1]

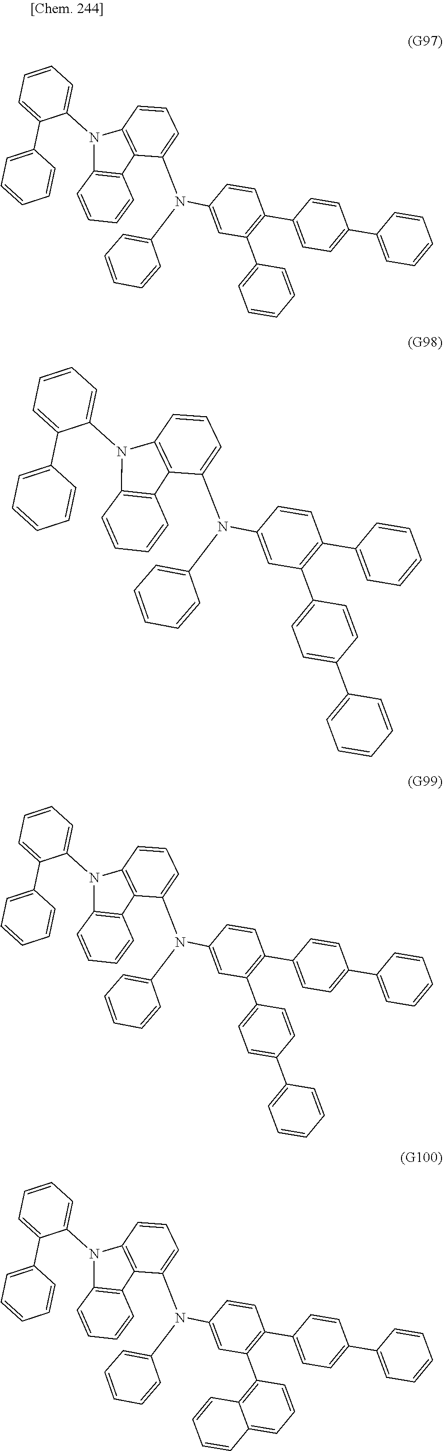 [SMILES: c1ccc(-c2ccc(-c3ccc(N(c4ccccc4)c4cccc5c4c4ccccc4n5-c4ccccc4-c4ccccc4)cc3-c3ccccc3)cc2)cc1 ; c1ccc(-c2ccc(-c3cc(N(c4ccccc4)c4cccc5c4c4ccccc4n5-c4ccccc4-c4ccccc4)ccc3-c3ccccc3)cc2)cc1 ; c1ccc(-c2ccc(-c3ccc(N(c4ccccc4)c4cccc5c4c4ccccc4n5-c4ccccc4-c4ccccc4)cc3-c3ccc(-c4ccccc4)cc3)cc2)cc1 ; c1ccc(-c2ccc(-c3ccc(N(c4ccccc4)c4cccc5c4c4ccccc4n5-c4ccccc4-c4ccccc4)cc3-c3cccc4ccccc34)cc2)cc1]

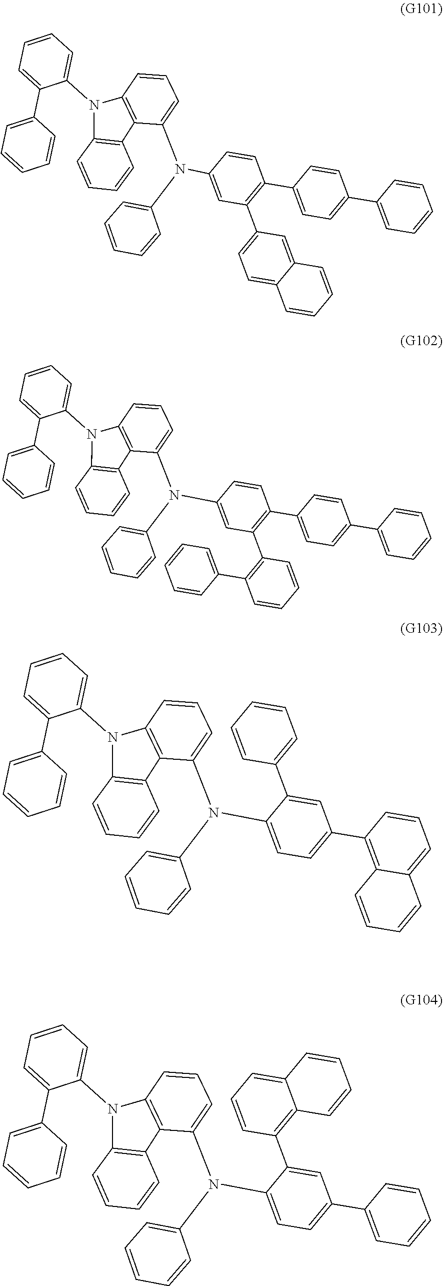 [SMILES: c1ccc(-c2ccc(-c3ccc(N(c4ccccc4)c4cccc5c4c4ccccc4n5-c4ccccc4-c4ccccc4)cc3-c3ccc4ccccc4c3)cc2)cc1 ; c1ccc(-c2ccc(-c3ccc(N(c4ccccc4)c4cccc5c4c4ccccc4n5-c4ccccc4-c4ccccc4)cc3-c3ccccc3-c3ccccc3)cc2)cc1 ; c1ccc(-c2cc(-c3cccc4ccccc34)ccc2N(c2ccccc2)c2cccc3c2c2ccccc2n3-c2ccccc2-c2ccccc2)cc1 ; c1ccc(-c2ccc(N(c3ccccc3)c3cccc4c3c3ccccc3n4-c3ccccc3-c3ccccc3)c(-c3cccc4ccccc34)c2)cc1]

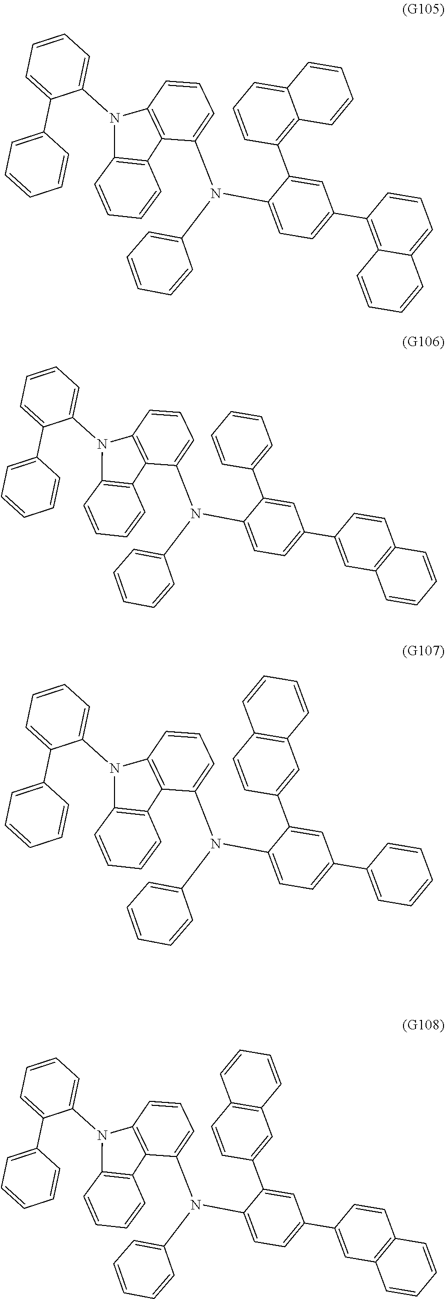 [SMILES: c1ccc(-c2ccccc2-n2c3ccccc3c3c(N(c4ccccc4)c4ccc(-c5cccc6ccccc56)cc4-c4cccc5ccccc45)cccc32)cc1 ; c1ccc(-c2cc(-c3ccc4ccccc4c3)ccc2N(c2ccccc2)c2cccc3c2c2ccccc2n3-c2ccccc2-c2ccccc2)cc1 ; c1ccc(-c2ccc(N(c3ccccc3)c3cccc4c3c3ccccc3n4-c3ccccc3-c3ccccc3)c(-c3ccc4ccccc4c3)c2)cc1 ; c1ccc(-c2ccccc2-n2c3ccccc3c3c(N(c4ccccc4)c4ccc(-c5ccc6ccccc6c5)cc4-c4ccc5ccccc5c4)cccc32)cc1]

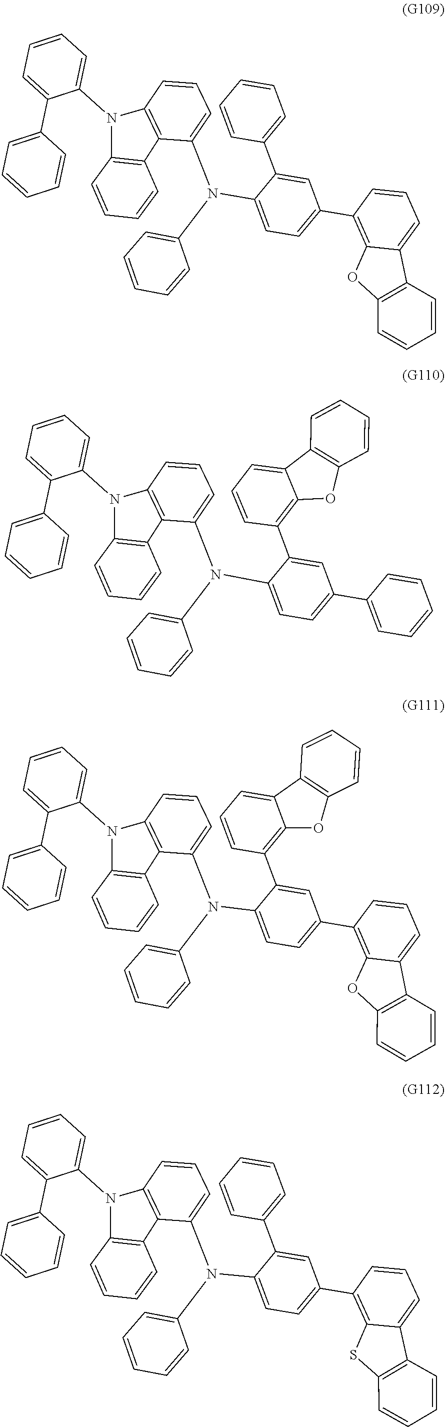 [SMILES: c1ccc(-c2cc(-c3cccc4c3oc3ccccc34)ccc2N(c2ccccc2)c2cccc3c2c2ccccc2n3-c2ccccc2-c2ccccc2)cc1 ; c1ccc(-c2ccc(N(c3ccccc3)c3cccc4c3c3ccccc3n4-c3ccccc3-c3ccccc3)c(-c3cccc4c3oc3ccccc34)c2)cc1 ; c1ccc(-c2ccccc2-n2c3ccccc3c3c(N(c4ccccc4)c4ccc(-c5cccc6c5oc5ccccc56)cc4-c4cccc5c4oc4ccccc45)cccc32)cc1 ; c1ccc(-c2cc(-c3cccc4c3sc3ccccc34)ccc2N(c2ccccc2)c2cccc3c2c2ccccc2n3-c2ccccc2-c2ccccc2)cc1]

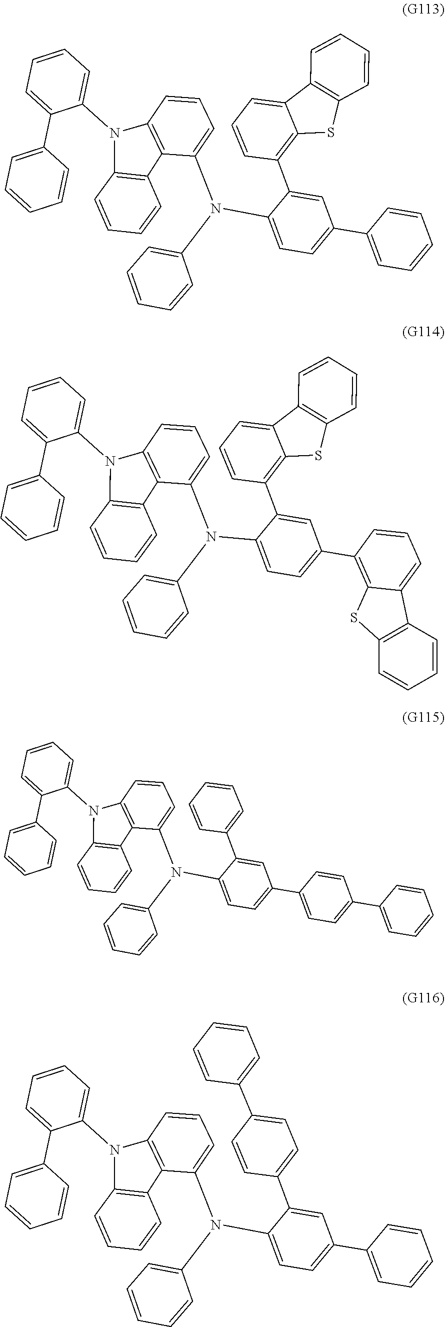 [SMILES: c1ccc(-c2ccc(N(c3ccccc3)c3cccc4c3c3ccccc3n4-c3ccccc3-c3ccccc3)c(-c3cccc4c3sc3ccccc34)c2)cc1 ; c1ccc(-c2ccccc2-n2c3ccccc3c3c(N(c4ccccc4)c4ccc(-c5cccc6c5sc5ccccc56)cc4-c4cccc5c4sc4ccccc45)cccc32)cc1 ; c1ccc(-c2ccc(-c3ccc(N(c4ccccc4)c4cccc5c4c4ccccc4n5-c4ccccc4-c4ccccc4)c(-c4ccccc4)c3)cc2)cc1 ; c1ccc(-c2ccc(-c3cc(-c4ccccc4)ccc3N(c3ccccc3)c3cccc4c3c3ccccc3n4-c3ccccc3-c3ccccc3)cc2)cc1]

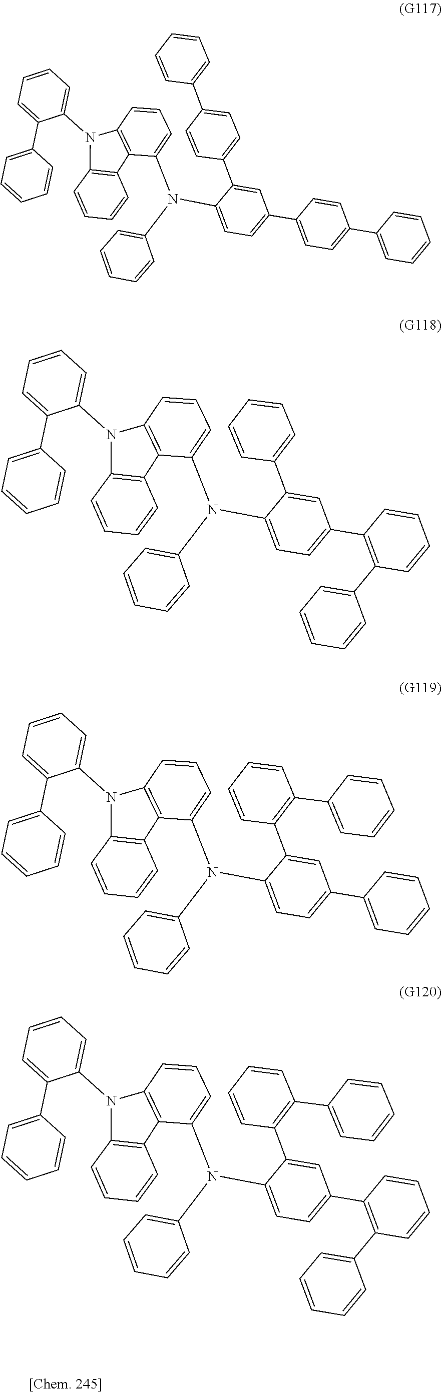 [SMILES: c1ccc(-c2ccc(-c3ccc(N(c4ccccc4)c4cccc5c4c4ccccc4n5-c4ccccc4-c4ccccc4)c(-c4ccc(-c5ccccc5)cc4)c3)cc2)cc1 ; c1ccc(-c2ccccc2-c2ccc(N(c3ccccc3)c3cccc4c3c3ccccc3n4-c3ccccc3-c3ccccc3)c(-c3ccccc3)c2)cc1 ; c1ccc(-c2ccc(N(c3ccccc3)c3cccc4c3c3ccccc3n4-c3ccccc3-c3ccccc3)c(-c3ccccc3-c3ccccc3)c2)cc1 ; c1ccc(-c2ccccc2-c2ccc(N(c3ccccc3)c3cccc4c3c3ccccc3n4-c3ccccc3-c3ccccc3)c(-c3ccccc3-c3ccccc3)c2)cc1]

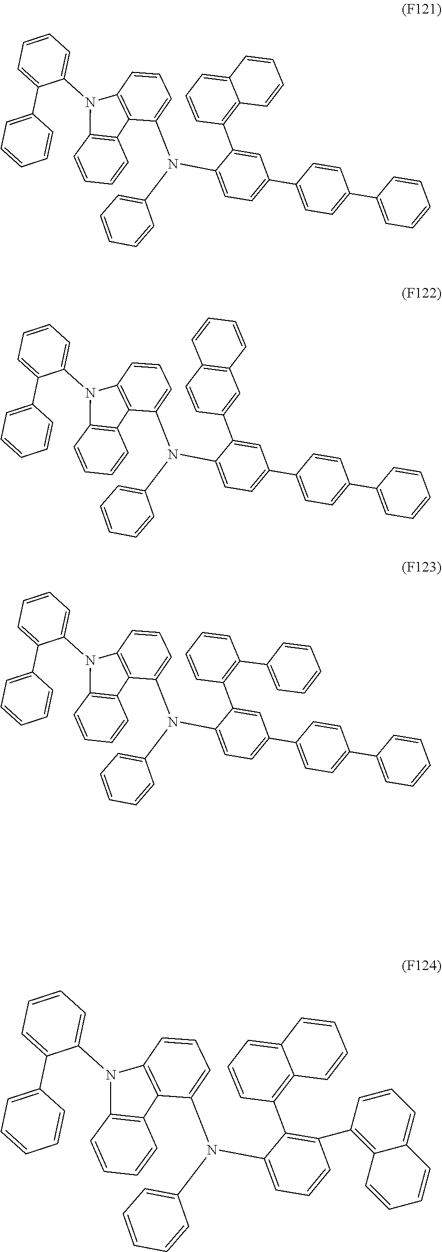 [SMILES: c1ccc(-c2ccc(-c3ccc(N(c4ccccc4)c4cccc5c4c4ccccc4n5-c4ccccc4-c4ccccc4)c(-c4cccc5ccccc45)c3)cc2)cc1 ; c1ccc(-c2ccc(-c3ccc(N(c4ccccc4)c4cccc5c4c4ccccc4n5-c4ccccc4-c4ccccc4)c(-c4ccc5ccccc5c4)c3)cc2)cc1 ; c1ccc(-c2ccc(-c3ccc(N(c4ccccc4)c4cccc5c4c4ccccc4n5-c4ccccc4-c4ccccc4)c(-c4ccccc4-c4ccccc4)c3)cc2)cc1 ; c1ccc(-c2ccccc2-n2c3ccccc3c3c(N(c4ccccc4)c4cccc(-c5cccc6ccccc56)c4-c4cccc5ccccc45)cccc32)cc1]

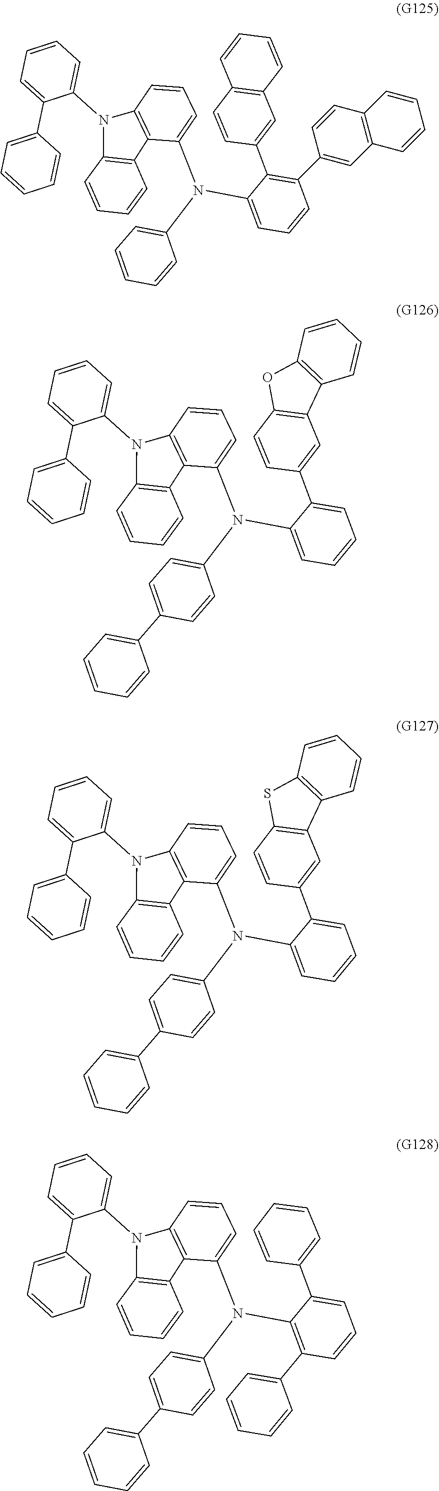 [SMILES: c1ccc(-c2ccccc2-n2c3ccccc3c3c(N(c4ccccc4)c4cccc(-c5ccc6ccccc6c5)c4-c4ccc5ccccc5c4)cccc32)cc1 ; c1ccc(-c2ccc(N(c3ccccc3-c3ccc4oc5ccccc5c4c3)c3cccc4c3c3ccccc3n4-c3ccccc3-c3ccccc3)cc2)cc1 ; c1ccc(-c2ccc(N(c3ccccc3-c3ccc4sc5ccccc5c4c3)c3cccc4c3c3ccccc3n4-c3ccccc3-c3ccccc3)cc2)cc1 ; c1ccc(-c2ccc(N(c3c(-c4ccccc4)cccc3-c3ccccc3)c3cccc4c3c3ccccc3n4-c3ccccc3-c3ccccc3)cc2)cc1]

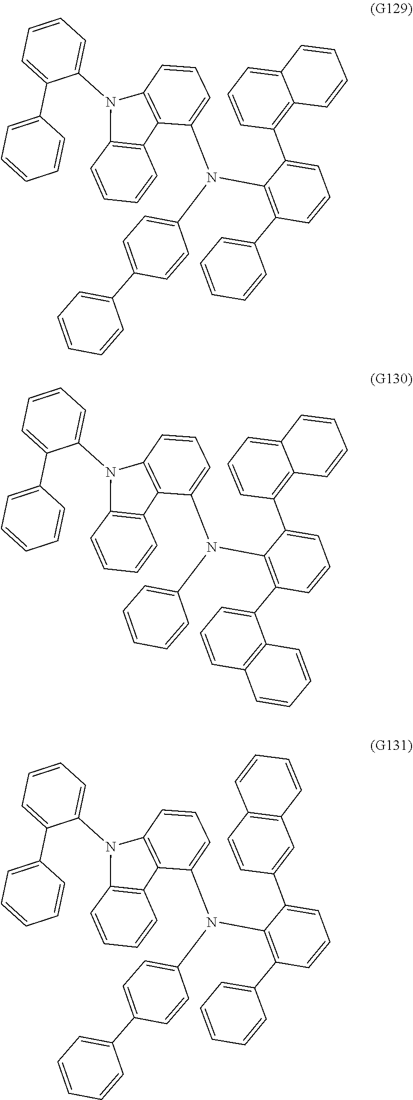

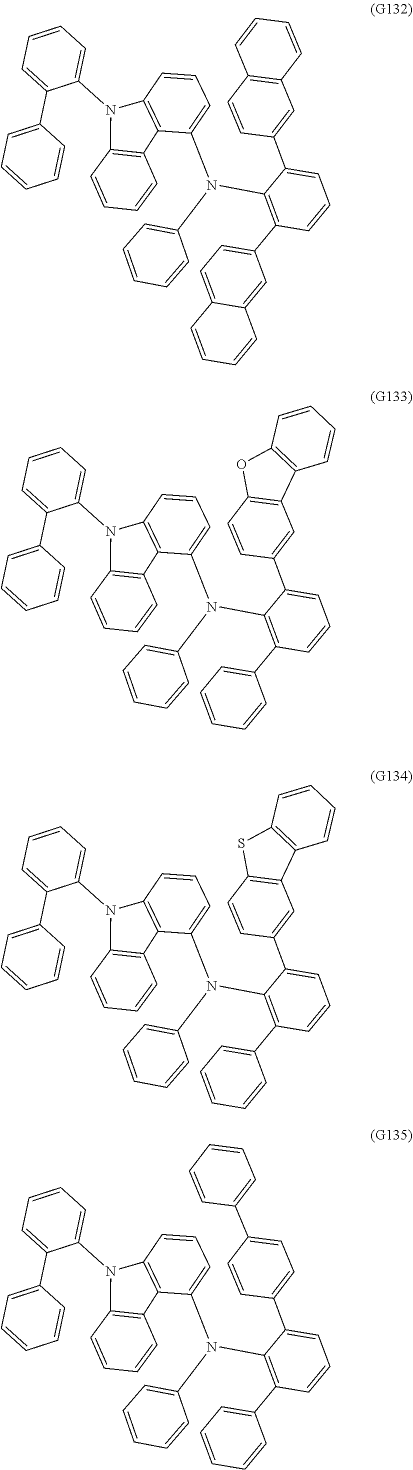

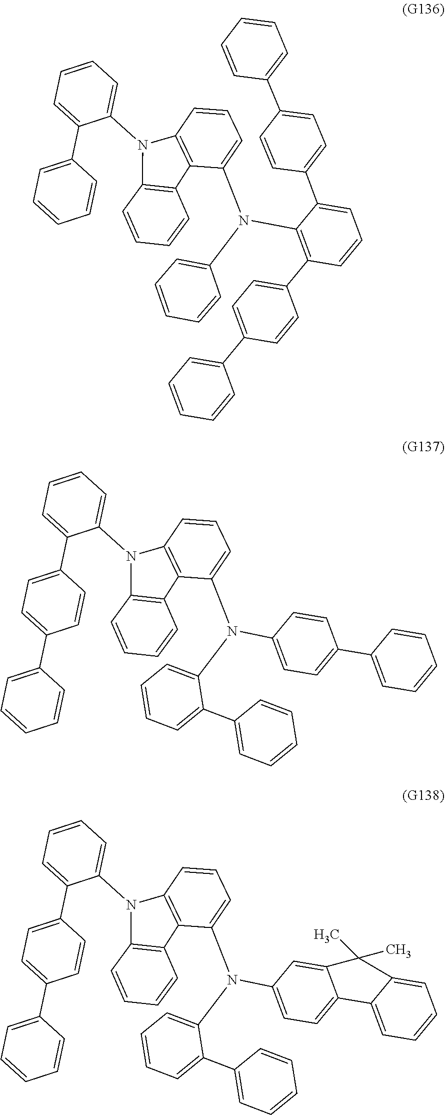 [SMILES: c1ccc(-c2ccc(-c3cccc(-c4ccc(-c5ccccc5)cc4)c3N(c3ccccc3)c3cccc4c3c3ccccc3n4-c3ccccc3-c3ccccc3)cc2)cc1 ; c1ccc(-c2ccc(-c3ccccc3-n3c4ccccc4c4c(N(c5ccc(-c6ccccc6)cc5)c5ccccc5-c5ccccc5)cccc43)cc2)cc1 ; CC1(C)c2ccccc2-c2ccc(N(c3ccccc3-c3ccccc3)c3cccc4c3c3ccccc3n4-c3ccccc3-c3ccc(-c4ccccc4)cc3)cc21]

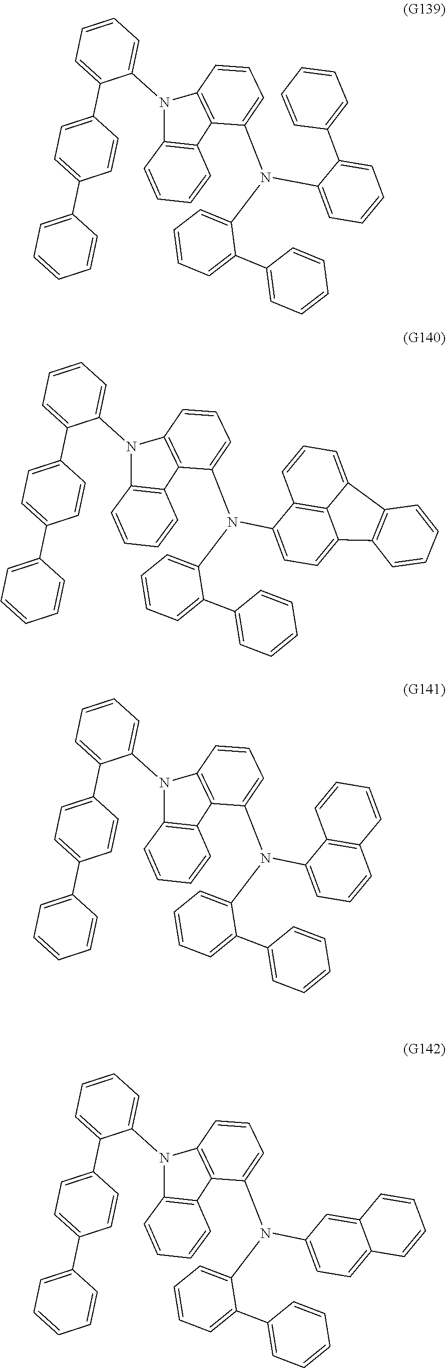 [SMILES: c1ccc(-c2ccc(-c3ccccc3-n3c4ccccc4c4c(N(c5ccccc5-c5ccccc5)c5ccccc5-c5ccccc5)cccc43)cc2)cc1 ; c1ccc(-c2ccc(-c3ccccc3-n3c4ccccc4c4c(N(c5ccccc5-c5ccccc5)c5ccc6c7c(cccc57)-c5ccccc5-6)cccc43)cc2)cc1 ; c1ccc(-c2ccc(-c3ccccc3-n3c4ccccc4c4c(N(c5ccccc5-c5ccccc5)c5cccc6ccccc56)cccc43)cc2)cc1 ; c1ccc(-c2ccc(-c3ccccc3-n3c4ccccc4c4c(N(c5ccc6ccccc6c5)c5ccccc5-c5ccccc5)cccc43)cc2)cc1]

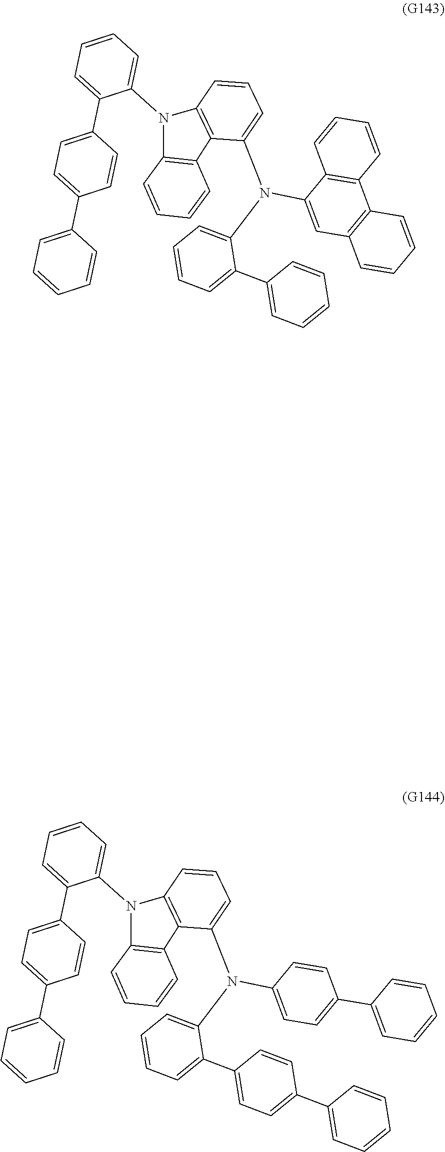 [SMILES: c1ccc(-c2ccc(-c3ccccc3-n3c4ccccc4c4c(N(c5ccccc5-c5ccccc5)c5cc6ccccc6c6ccccc56)cccc43)cc2)cc1 ; c1ccc(-c2ccc(-c3ccccc3N(c3ccc(-c4ccccc4)cc3)c3cccc4c3c3ccccc3n4-c3ccccc3-c3ccc(-c4ccccc4)cc3)cc2)cc1]

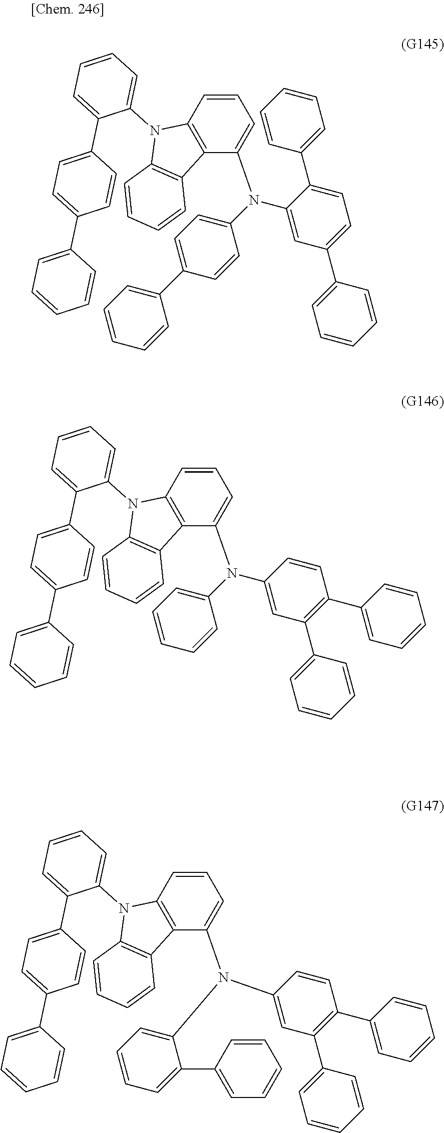 [SMILES: c1ccc(-c2ccc(-c3ccccc3-n3c4ccccc4c4c(N(c5ccc(-c6ccccc6)cc5)c5cc(-c6ccccc6)ccc5-c5ccccc5)cccc43)cc2)cc1 ; c1ccc(-c2ccc(-c3ccccc3-n3c4ccccc4c4c(N(c5ccccc5)c5ccc(-c6ccccc6)c(-c6ccccc6)c5)cccc43)cc2)cc1 ; c1ccc(-c2ccc(-c3ccccc3-n3c4ccccc4c4c(N(c5ccc(-c6ccccc6)c(-c6ccccc6)c5)c5ccccc5-c5ccccc5)cccc43)cc2)cc1]

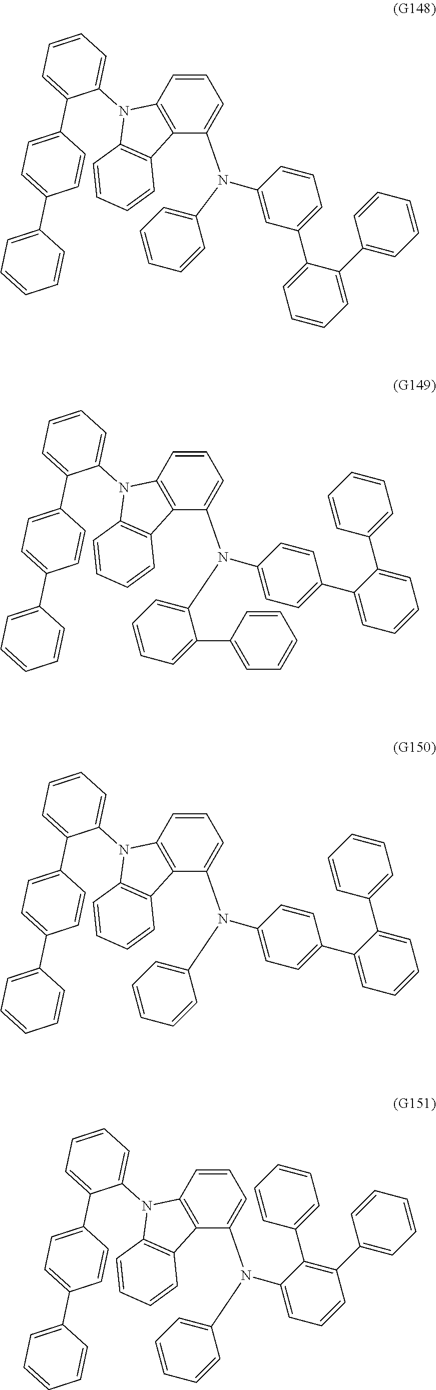 [SMILES: c1ccc(-c2ccc(-c3ccccc3-n3c4ccccc4c4c(N(c5ccccc5)c5cccc(-c6ccccc6-c6ccccc6)c5)cccc43)cc2)cc1 ; c1ccc(-c2ccc(-c3ccccc3-n3c4ccccc4c4c(N(c5ccc(-c6ccccc6-c6ccccc6)cc5)c5ccccc5-c5ccccc5)cccc43)cc2)cc1 ; c1ccc(-c2ccc(-c3ccccc3-n3c4ccccc4c4c(N(c5ccccc5)c5ccc(-c6ccccc6-c6ccccc6)cc5)cccc43)cc2)cc1 ; c1ccc(-c2ccc(-c3ccccc3-n3c4ccccc4c4c(N(c5ccccc5)c5cccc(-c6ccccc6)c5-c5ccccc5)cccc43)cc2)cc1]

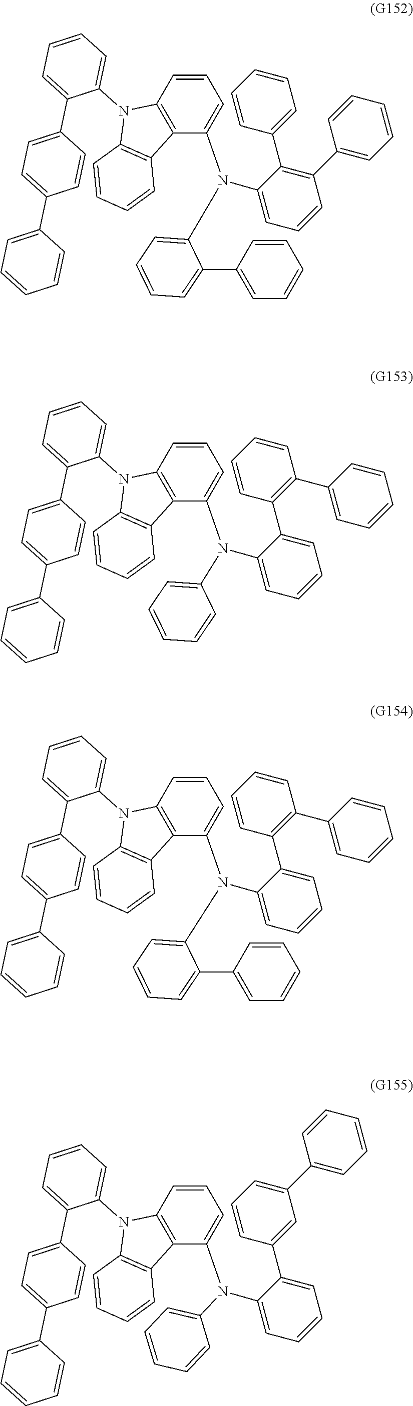 [SMILES: c1ccc(-c2ccc(-c3ccccc3-n3c4ccccc4c4c(N(c5ccccc5-c5ccccc5)c5cccc(-c6ccccc6)c5-c5ccccc5)cccc43)cc2)cc1 ; c1ccc(-c2ccc(-c3ccccc3-n3c4ccccc4c4c(N(c5ccccc5)c5ccccc5-c5ccccc5-c5ccccc5)cccc43)cc2)cc1 ; c1ccc(-c2ccc(-c3ccccc3-n3c4ccccc4c4c(N(c5ccccc5-c5ccccc5)c5ccccc5-c5ccccc5-c5ccccc5)cccc43)cc2)cc1 ; c1ccc(-c2ccc(-c3ccccc3-n3c4ccccc4c4c(N(c5ccccc5)c5ccccc5-c5cccc(-c6ccccc6)c5)cccc43)cc2)cc1]

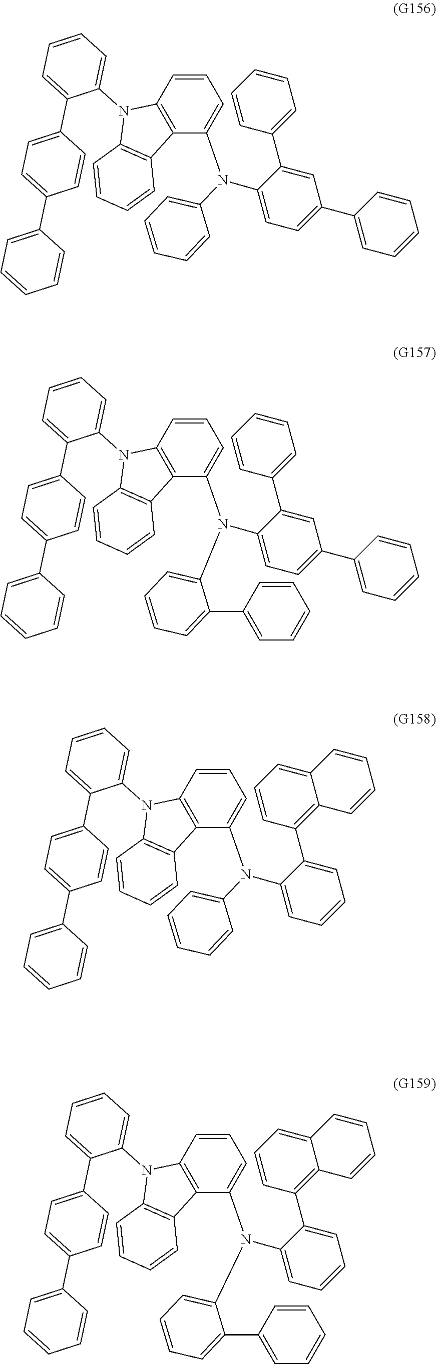 [SMILES: c1ccc(-c2ccc(-c3ccccc3-n3c4ccccc4c4c(N(c5ccccc5)c5ccc(-c6ccccc6)cc5-c5ccccc5)cccc43)cc2)cc1 ; c1ccc(-c2ccc(-c3ccccc3-n3c4ccccc4c4c(N(c5ccccc5-c5ccccc5)c5ccc(-c6ccccc6)cc5-c5ccccc5)cccc43)cc2)cc1 ; c1ccc(-c2ccc(-c3ccccc3-n3c4ccccc4c4c(N(c5ccccc5)c5ccccc5-c5cccc6ccccc56)cccc43)cc2)cc1 ; c1ccc(-c2ccc(-c3ccccc3-n3c4ccccc4c4c(N(c5ccccc5-c5ccccc5)c5ccccc5-c5cccc6ccccc56)cccc43)cc2)cc1]

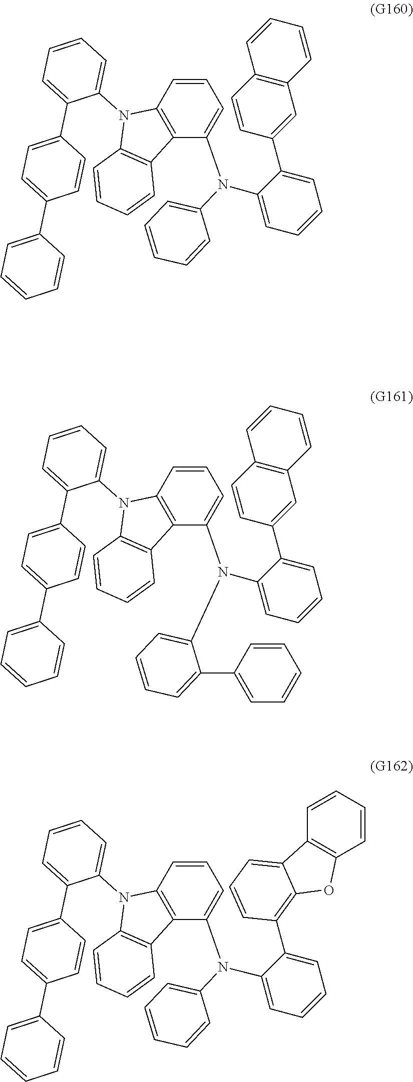 [SMILES: c1ccc(-c2ccc(-c3ccccc3-n3c4ccccc4c4c(N(c5ccccc5)c5ccccc5-c5ccc6ccccc6c5)cccc43)cc2)cc1 ; c1ccc(-c2ccc(-c3ccccc3-n3c4ccccc4c4c(N(c5ccccc5-c5ccccc5)c5ccccc5-c5ccc6ccccc6c5)cccc43)cc2)cc1 ; c1ccc(-c2ccc(-c3ccccc3-n3c4ccccc4c4c(N(c5ccccc5)c5ccccc5-c5cccc6c5oc5ccccc56)cccc43)cc2)cc1]

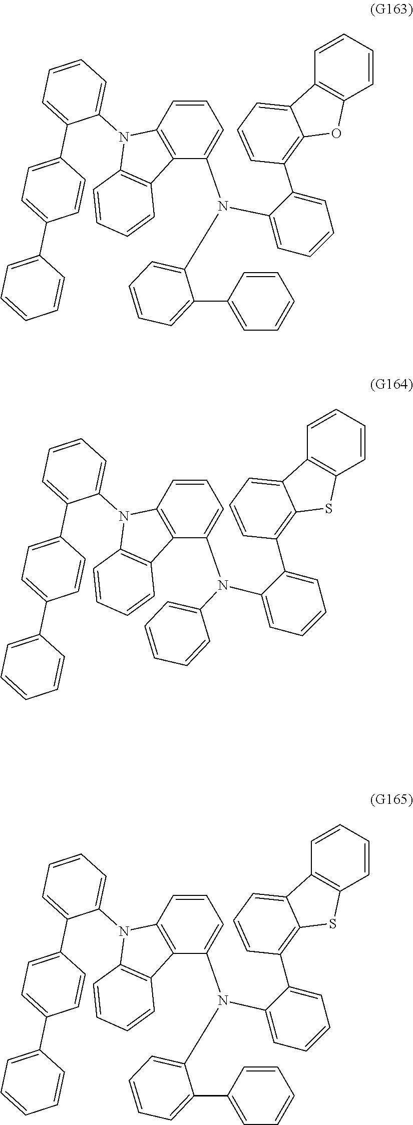 [SMILES: c1ccc(-c2ccc(-c3ccccc3-n3c4ccccc4c4c(N(c5ccccc5-c5ccccc5)c5ccccc5-c5cccc6c5oc5ccccc56)cccc43)cc2)cc1 ; c1ccc(-c2ccc(-c3ccccc3-n3c4ccccc4c4c(N(c5ccccc5)c5ccccc5-c5cccc6c5sc5ccccc56)cccc43)cc2)cc1 ; c1ccc(-c2ccc(-c3ccccc3-n3c4ccccc4c4c(N(c5ccccc5-c5ccccc5)c5ccccc5-c5cccc6c5sc5ccccc56)cccc43)cc2)cc1]

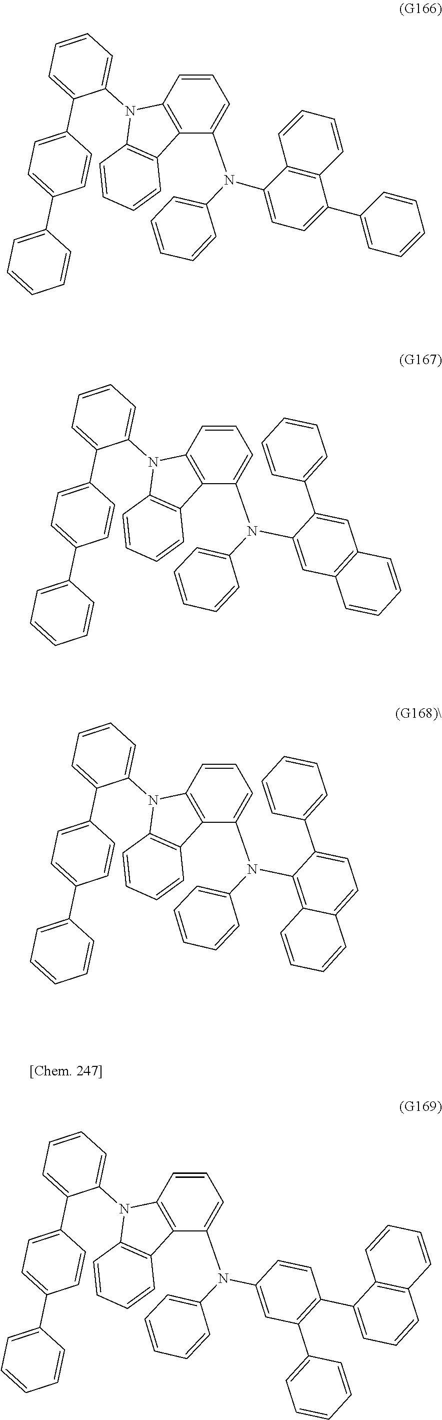 [SMILES: c1ccc(-c2ccc(-c3ccccc3-n3c4ccccc4c4c(N(c5ccccc5)c5ccc(-c6ccccc6)c6ccccc56)cccc43)cc2)cc1 ; c1ccc(-c2ccc(-c3ccccc3-n3c4ccccc4c4c(N(c5ccccc5)c5cc6ccccc6cc5-c5ccccc5)cccc43)cc2)cc1 ; c1ccc(-c2ccc(-c3ccccc3-n3c4ccccc4c4c(N(c5ccccc5)c5c(-c6ccccc6)ccc6ccccc56)cccc43)cc2)cc1 ; c1ccc(-c2ccc(-c3ccccc3-n3c4ccccc4c4c(N(c5ccccc5)c5ccc(-c6cccc7ccccc67)c(-c6ccccc6)c5)cccc43)cc2)cc1]

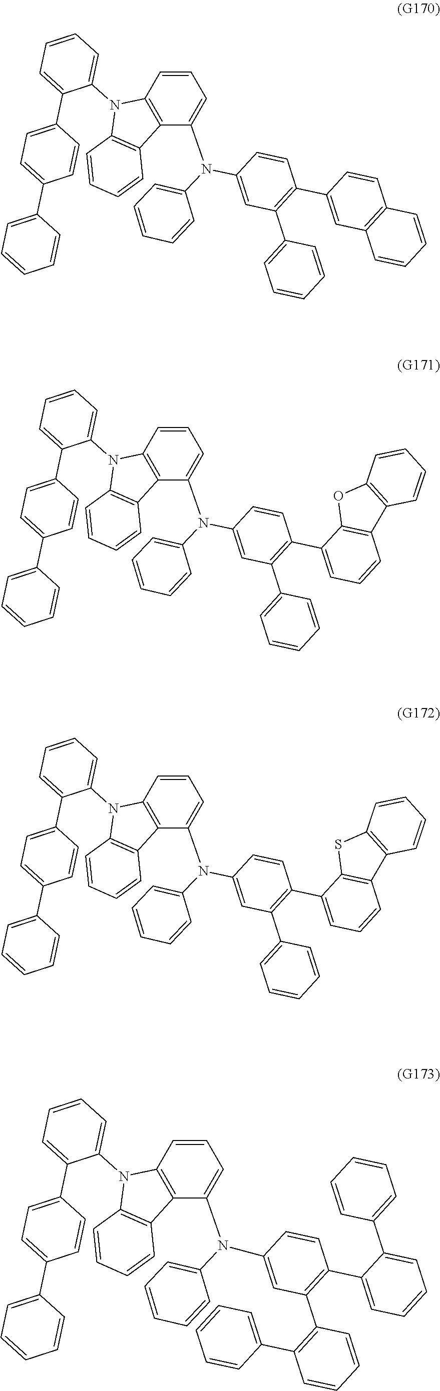 [SMILES: c1ccc(-c2ccc(-c3ccccc3-n3c4ccccc4c4c(N(c5ccccc5)c5ccc(-c6ccc7ccccc7c6)c(-c6ccccc6)c5)cccc43)cc2)cc1 ; c1ccc(-c2ccc(-c3ccccc3-n3c4ccccc4c4c(N(c5ccccc5)c5ccc(-c6cccc7c6oc6ccccc67)c(-c6ccccc6)c5)cccc43)cc2)cc1 ; c1ccc(-c2ccc(-c3ccccc3-n3c4ccccc4c4c(N(c5ccccc5)c5ccc(-c6cccc7c6sc6ccccc67)c(-c6ccccc6)c5)cccc43)cc2)cc1 ; c1ccc(-c2ccc(-c3ccccc3-n3c4ccccc4c4c(N(c5ccccc5)c5ccc(-c6ccccc6-c6ccccc6)c(-c6ccccc6-c6ccccc6)c5)cccc43)cc2)cc1]

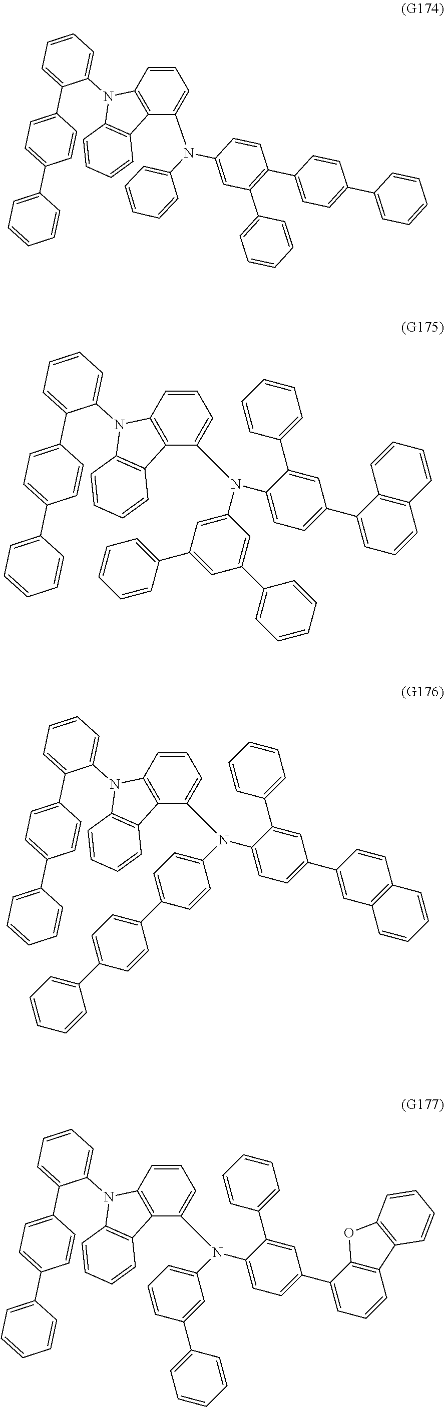 [SMILES: c1ccc(-c2ccc(-c3ccc(N(c4ccccc4)c4cccc5c4c4ccccc4n5-c4ccccc4-c4ccc(-c5ccccc5)cc4)cc3-c3ccccc3)cc2)cc1 ; c1ccc(-c2ccc(-c3ccccc3-n3c4ccccc4c4c(N(c5cc(-c6ccccc6)cc(-c6ccccc6)c5)c5ccc(-c6cccc7ccccc67)cc5-c5ccccc5)cccc43)cc2)cc1 ; c1ccc(-c2ccc(-c3ccc(N(c4ccc(-c5ccc6ccccc6c5)cc4-c4ccccc4)c4cccc5c4c4ccccc4n5-c4ccccc4-c4ccc(-c5ccccc5)cc4)cc3)cc2)cc1 ; c1ccc(-c2ccc(-c3ccccc3-n3c4ccccc4c4c(N(c5cccc(-c6ccccc6)c5)c5ccc(-c6cccc7c6oc6ccccc67)cc5-c5ccccc5)cccc43)cc2)cc1]

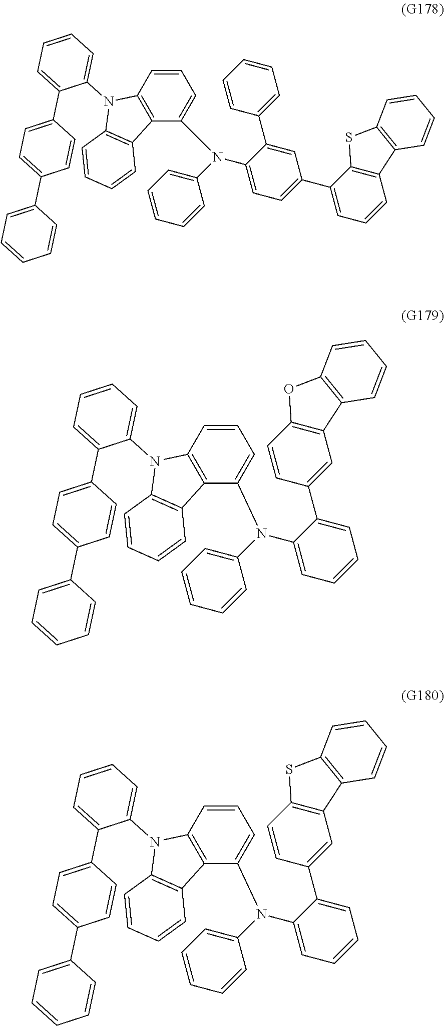 [SMILES: c1ccc(-c2ccc(-c3ccccc3-n3c4ccccc4c4c(N(c5ccccc5)c5ccc(-c6cccc7c6sc6ccccc67)cc5-c5ccccc5)cccc43)cc2)cc1 ; c1ccc(-c2ccc(-c3ccccc3-n3c4ccccc4c4c(N(c5ccccc5)c5ccccc5-c5ccc6oc7ccccc7c6c5)cccc43)cc2)cc1 ; c1ccc(-c2ccc(-c3ccccc3-n3c4ccccc4c4c(N(c5ccccc5)c5ccccc5-c5ccc6sc7ccccc7c6c5)cccc43)cc2)cc1]

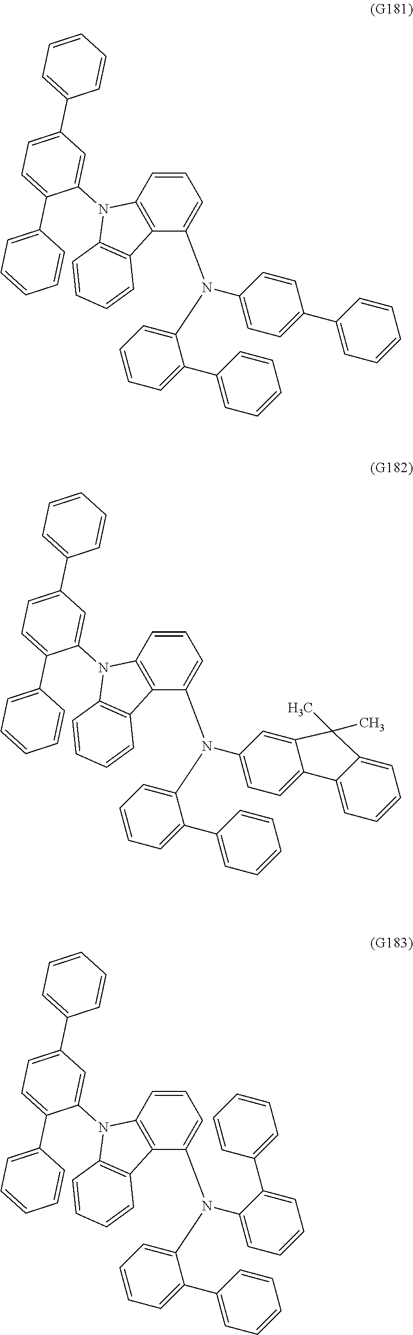 [SMILES: c1ccc(-c2ccc(N(c3ccccc3-c3ccccc3)c3cccc4c3c3ccccc3n4-c3cc(-c4ccccc4)ccc3-c3ccccc3)cc2)cc1 ; CC1(C)c2ccccc2-c2ccc(N(c3ccccc3-c3ccccc3)c3cccc4c3c3ccccc3n4-c3cc(-c4ccccc4)ccc3-c3ccccc3)cc21 ; c1ccc(-c2ccc(-c3ccccc3)c(-n3c4ccccc4c4c(N(c5ccccc5-c5ccccc5)c5ccccc5-c5ccccc5)cccc43)c2)cc1]

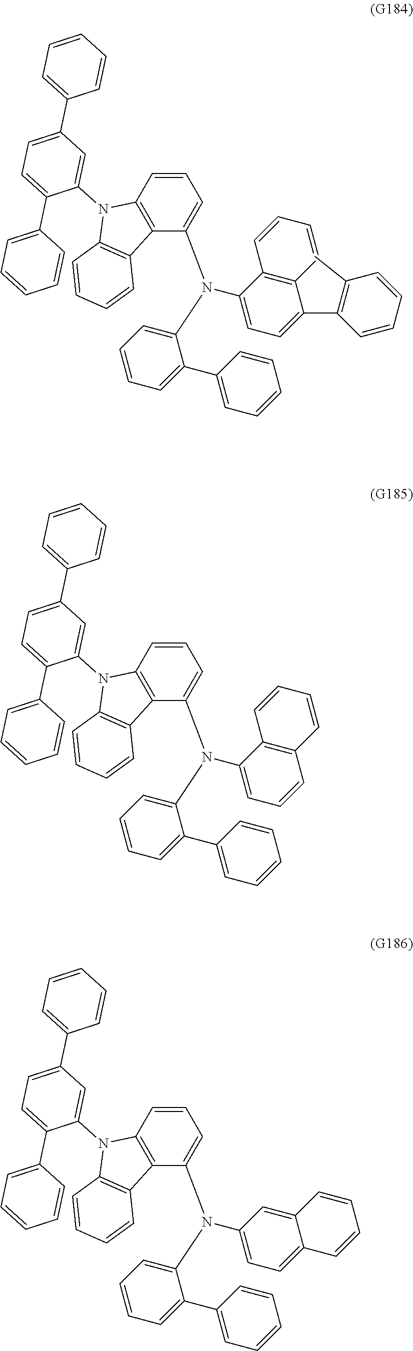

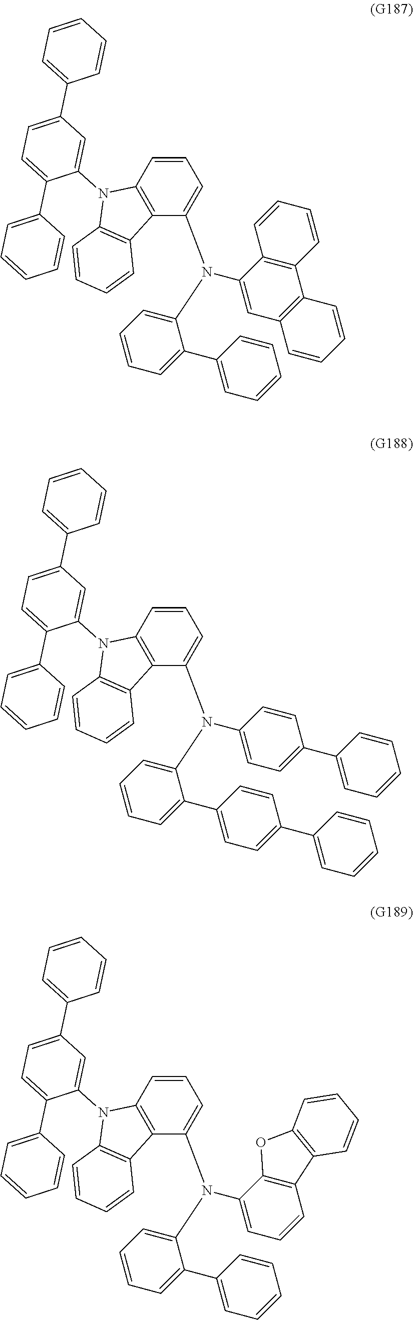 [SMILES: c1ccc(-c2ccc(-c3ccccc3)c(-n3c4ccccc4c4c(N(c5ccccc5-c5ccccc5)c5cc6ccccc6c6ccccc56)cccc43)c2)cc1 ; c1ccc(-c2ccc(-c3ccccc3N(c3ccc(-c4ccccc4)cc3)c3cccc4c3c3ccccc3n4-c3cc(-c4ccccc4)ccc3-c3ccccc3)cc2)cc1 ; c1ccc(-c2ccc(-c3ccccc3)c(-n3c4ccccc4c4c(N(c5ccccc5-c5ccccc5)c5cccc6c5oc5ccccc56)cccc43)c2)cc1]

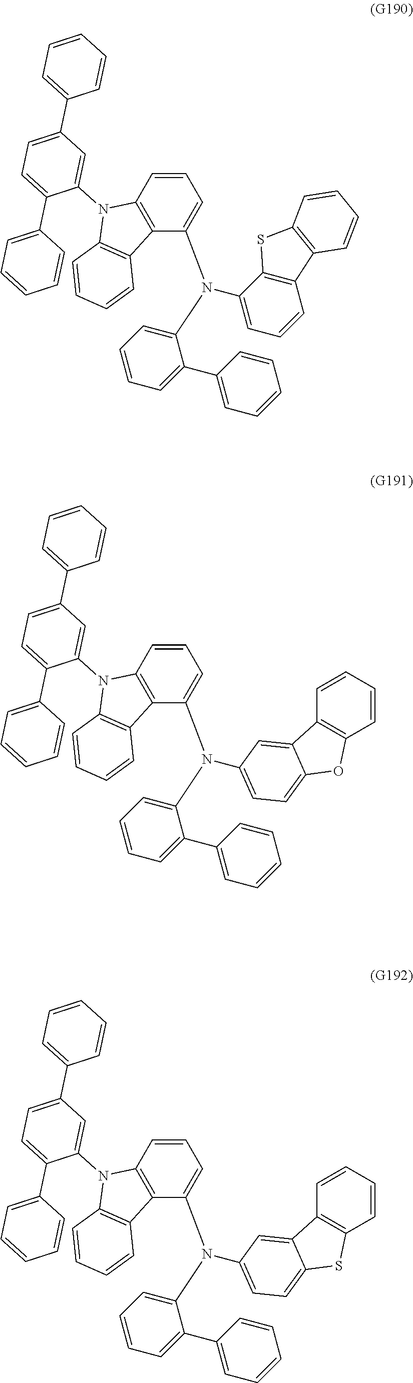 [SMILES: c1ccc(-c2ccc(-c3ccccc3)c(-n3c4ccccc4c4c(N(c5ccccc5-c5ccccc5)c5cccc6c5sc5ccccc56)cccc43)c2)cc1 ; c1ccc(-c2ccc(-c3ccccc3)c(-n3c4ccccc4c4c(N(c5ccc6oc7ccccc7c6c5)c5ccccc5-c5ccccc5)cccc43)c2)cc1 ; c1ccc(-c2ccc(-c3ccccc3)c(-n3c4ccccc4c4c(N(c5ccc6sc7ccccc7c6c5)c5ccccc5-c5ccccc5)cccc43)c2)cc1]

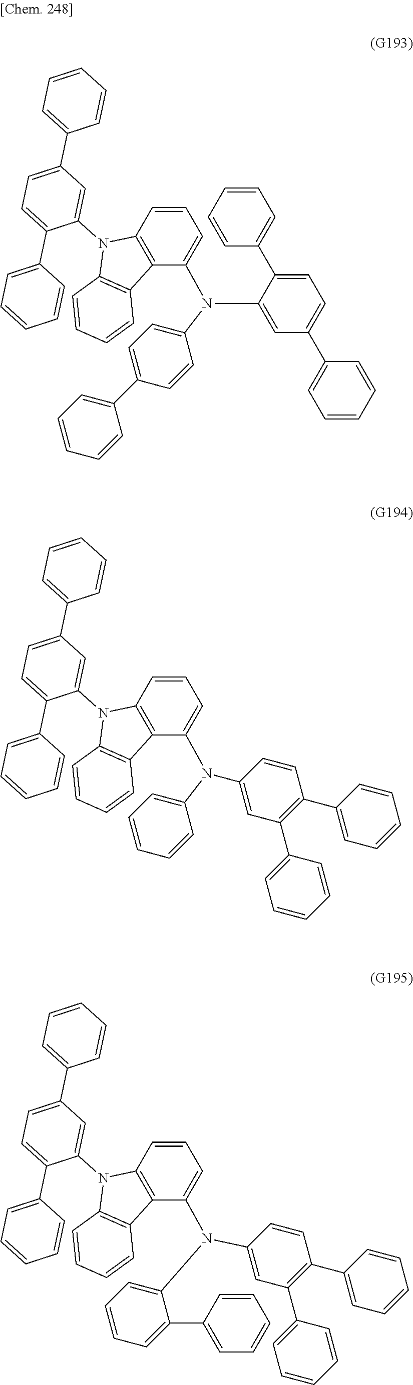 [SMILES: c1ccc(-c2ccc(N(c3cc(-c4ccccc4)ccc3-c3ccccc3)c3cccc4c3c3ccccc3n4-c3cc(-c4ccccc4)ccc3-c3ccccc3)cc2)cc1 ; c1ccc(-c2ccc(-c3ccccc3)c(-n3c4ccccc4c4c(N(c5ccccc5)c5ccc(-c6ccccc6)c(-c6ccccc6)c5)cccc43)c2)cc1 ; c1ccc(-c2ccc(-c3ccccc3)c(-n3c4ccccc4c4c(N(c5ccc(-c6ccccc6)c(-c6ccccc6)c5)c5ccccc5-c5ccccc5)cccc43)c2)cc1]

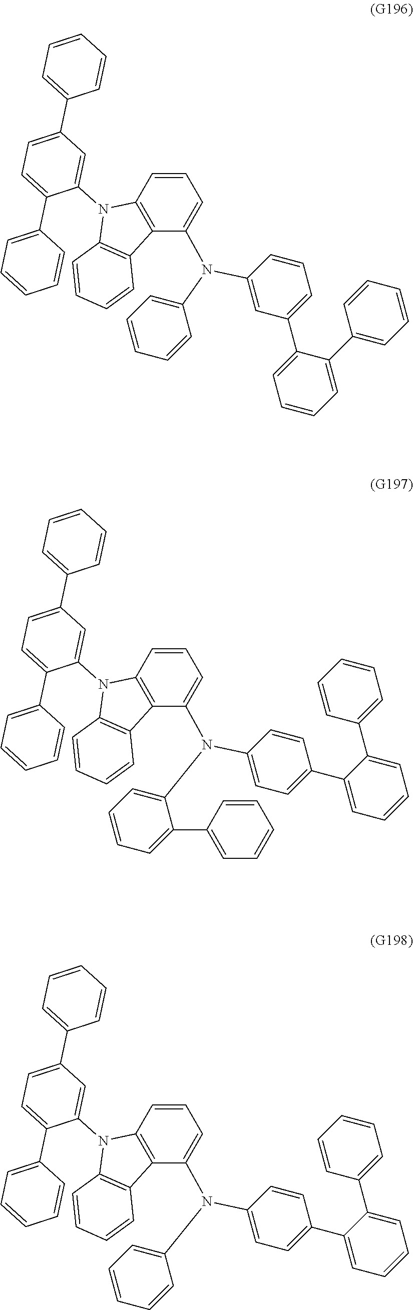 [SMILES: c1ccc(-c2ccc(-c3ccccc3)c(-n3c4ccccc4c4c(N(c5ccccc5)c5cccc(-c6ccccc6-c6ccccc6)c5)cccc43)c2)cc1 ; c1ccc(-c2ccc(-c3ccccc3)c(-n3c4ccccc4c4c(N(c5ccc(-c6ccccc6-c6ccccc6)cc5)c5ccccc5-c5ccccc5)cccc43)c2)cc1 ; c1ccc(-c2ccc(-c3ccccc3)c(-n3c4ccccc4c4c(N(c5ccccc5)c5ccc(-c6ccccc6-c6ccccc6)cc5)cccc43)c2)cc1]

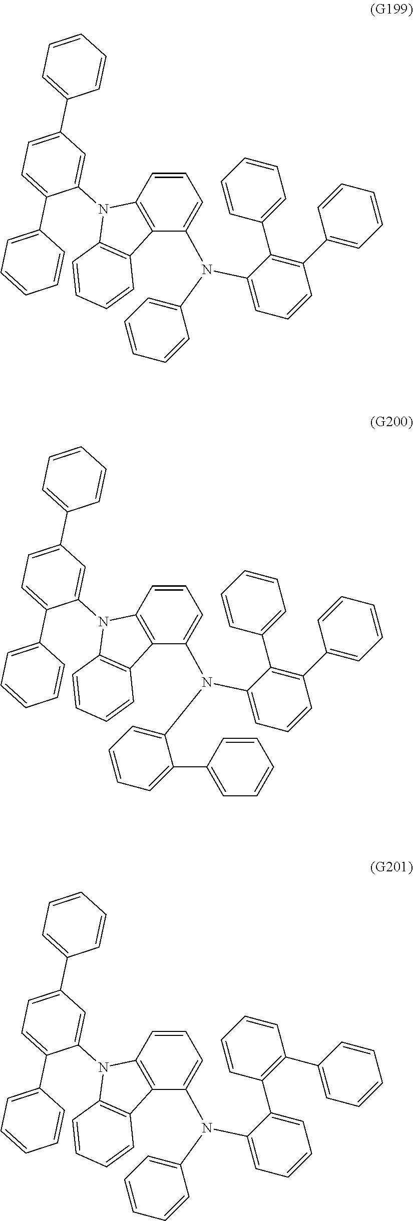 [SMILES: c1ccc(-c2ccc(-c3ccccc3)c(-n3c4ccccc4c4c(N(c5ccccc5)c5cccc(-c6ccccc6)c5-c5ccccc5)cccc43)c2)cc1 ; c1ccc(-c2ccc(-c3ccccc3)c(-n3c4ccccc4c4c(N(c5ccccc5-c5ccccc5)c5cccc(-c6ccccc6)c5-c5ccccc5)cccc43)c2)cc1 ; c1ccc(-c2ccc(-c3ccccc3)c(-n3c4ccccc4c4c(N(c5ccccc5)c5ccccc5-c5ccccc5-c5ccccc5)cccc43)c2)cc1]

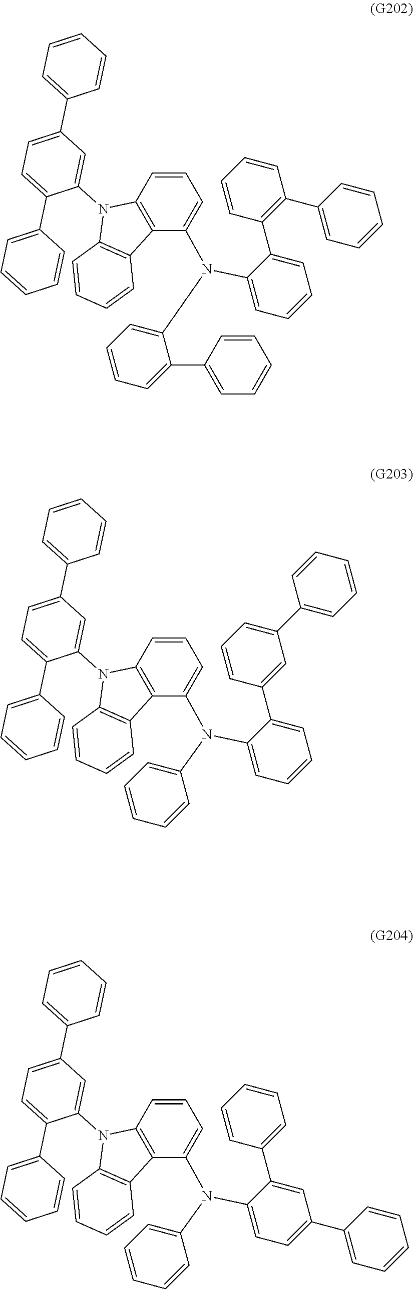 [SMILES: c1ccc(-c2ccc(-c3ccccc3)c(-n3c4ccccc4c4c(N(c5ccccc5-c5ccccc5)c5ccccc5-c5ccccc5-c5ccccc5)cccc43)c2)cc1 ; c1ccc(-c2cccc(-c3ccccc3N(c3ccccc3)c3cccc4c3c3ccccc3n4-c3cc(-c4ccccc4)ccc3-c3ccccc3)c2)cc1 ; c1ccc(-c2ccc(N(c3ccccc3)c3cccc4c3c3ccccc3n4-c3cc(-c4ccccc4)ccc3-c3ccccc3)c(-c3ccccc3)c2)cc1]

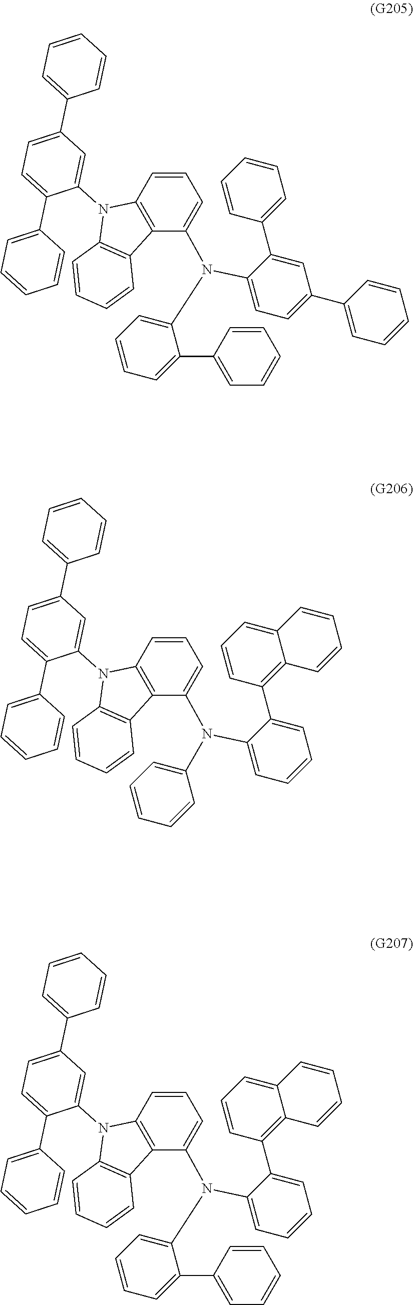 [SMILES: c1ccc(-c2ccc(N(c3ccccc3-c3ccccc3)c3cccc4c3c3ccccc3n4-c3cc(-c4ccccc4)ccc3-c3ccccc3)c(-c3ccccc3)c2)cc1 ; c1ccc(-c2ccc(-c3ccccc3)c(-n3c4ccccc4c4c(N(c5ccccc5)c5ccccc5-c5cccc6ccccc56)cccc43)c2)cc1 ; c1ccc(-c2ccc(-c3ccccc3)c(-n3c4ccccc4c4c(N(c5ccccc5-c5ccccc5)c5ccccc5-c5cccc6ccccc56)cccc43)c2)cc1]

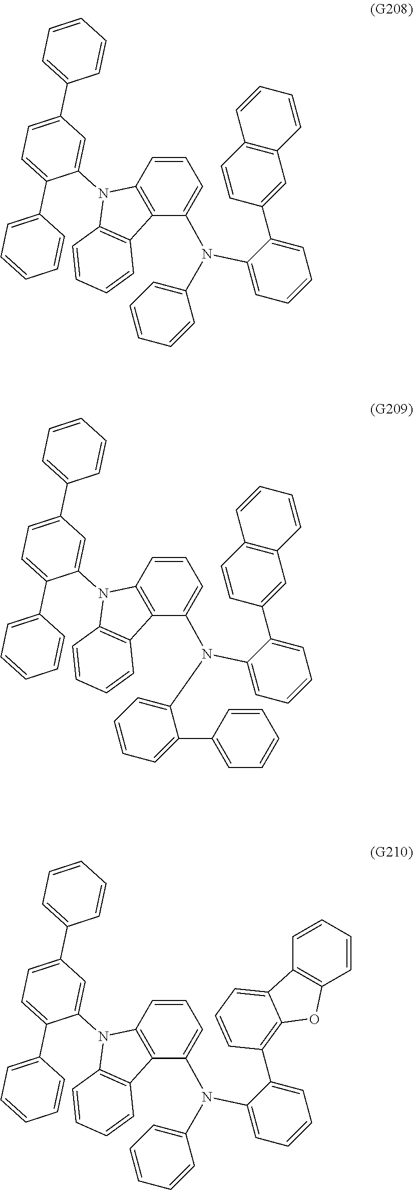 [SMILES: c1ccc(-c2ccc(-c3ccccc3)c(-n3c4ccccc4c4c(N(c5ccccc5)c5ccccc5-c5ccc6ccccc6c5)cccc43)c2)cc1 ; c1ccc(-c2ccc(-c3ccccc3)c(-n3c4ccccc4c4c(N(c5ccccc5-c5ccccc5)c5ccccc5-c5ccc6ccccc6c5)cccc43)c2)cc1 ; c1ccc(-c2ccc(-c3ccccc3)c(-n3c4ccccc4c4c(N(c5ccccc5)c5ccccc5-c5cccc6c5oc5ccccc56)cccc43)c2)cc1]

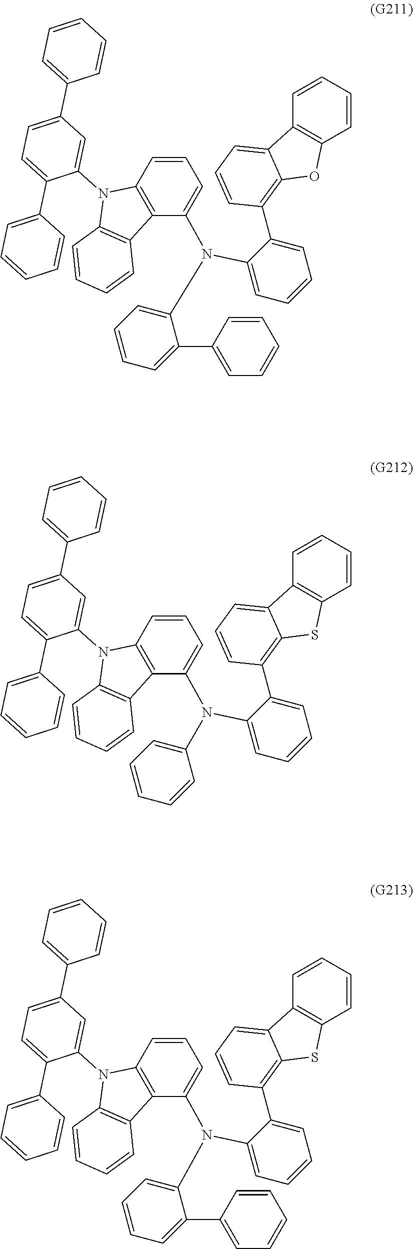 [SMILES: c1ccc(-c2ccc(-c3ccccc3)c(-n3c4ccccc4c4c(N(c5ccccc5-c5ccccc5)c5ccccc5-c5cccc6c5oc5ccccc56)cccc43)c2)cc1 ; c1ccc(-c2ccc(-c3ccccc3)c(-n3c4ccccc4c4c(N(c5ccccc5)c5ccccc5-c5cccc6c5sc5ccccc56)cccc43)c2)cc1 ; c1ccc(-c2ccc(-c3ccccc3)c(-n3c4ccccc4c4c(N(c5ccccc5-c5ccccc5)c5ccccc5-c5cccc6c5sc5ccccc56)cccc43)c2)cc1]

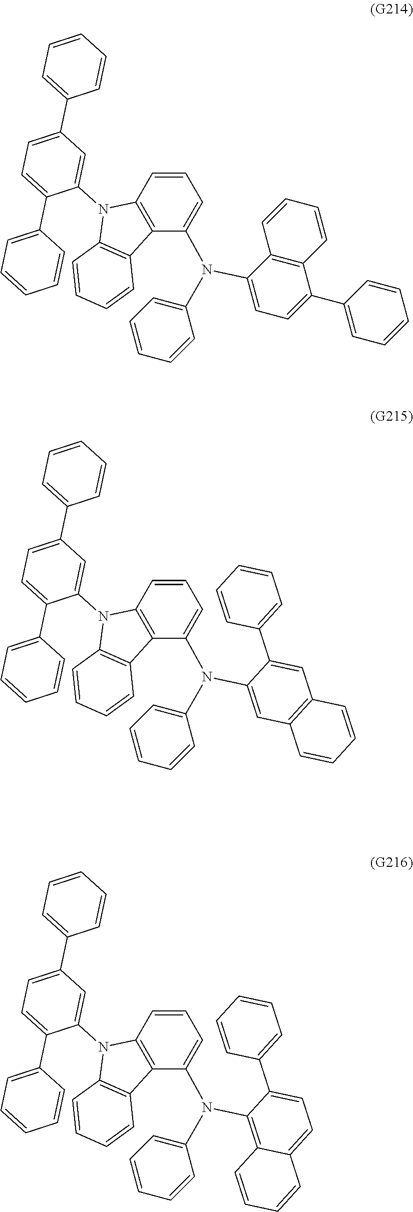 [SMILES: c1ccc(-c2ccc(-c3ccccc3)c(-n3c4ccccc4c4c(N(c5ccccc5)c5ccc(-c6ccccc6)c6ccccc56)cccc43)c2)cc1 ; c1ccc(-c2ccc(-c3ccccc3)c(-n3c4ccccc4c4c(N(c5ccccc5)c5cc6ccccc6cc5-c5ccccc5)cccc43)c2)cc1 ; c1ccc(-c2ccc(-c3ccccc3)c(-n3c4ccccc4c4c(N(c5ccccc5)c5c(-c6ccccc6)ccc6ccccc56)cccc43)c2)cc1]

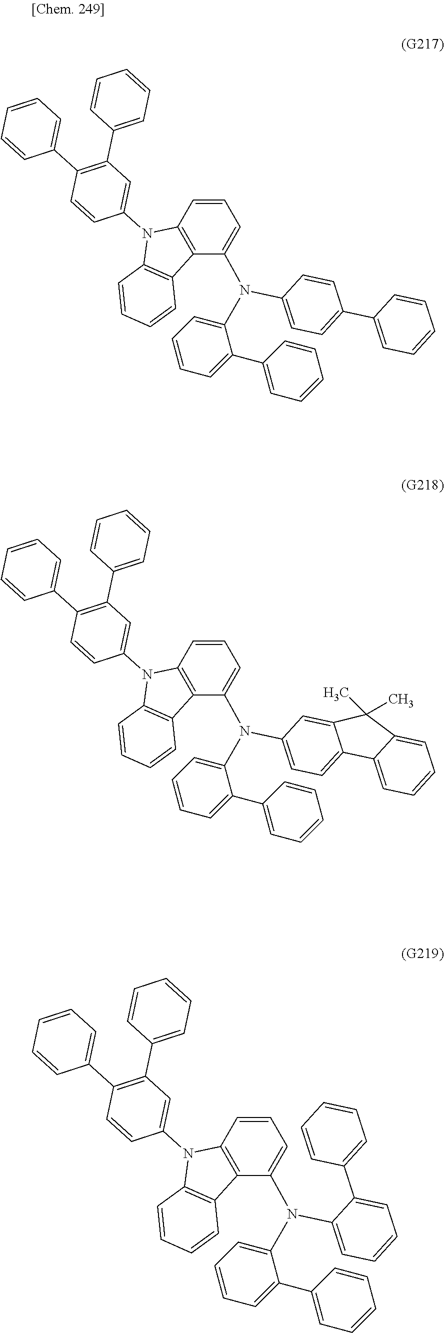 [SMILES: c1ccc(-c2ccc(N(c3ccccc3-c3ccccc3)c3cccc4c3c3ccccc3n4-c3ccc(-c4ccccc4)c(-c4ccccc4)c3)cc2)cc1 ; CC1(C)c2ccccc2-c2ccc(N(c3ccccc3-c3ccccc3)c3cccc4c3c3ccccc3n4-c3ccc(-c4ccccc4)c(-c4ccccc4)c3)cc21 ; c1ccc(-c2ccc(-n3c4ccccc4c4c(N(c5ccccc5-c5ccccc5)c5ccccc5-c5ccccc5)cccc43)cc2-c2ccccc2)cc1]

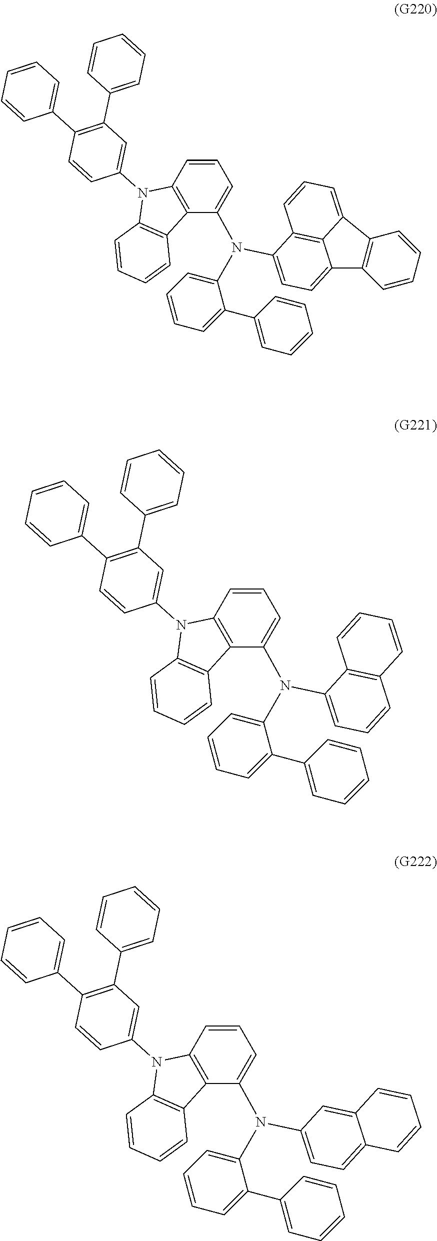 [SMILES: c1ccc(-c2ccc(-n3c4ccccc4c4c(N(c5ccccc5-c5ccccc5)c5ccc6c7c(cccc57)-c5ccccc5-6)cccc43)cc2-c2ccccc2)cc1 ; c1ccc(-c2ccc(-n3c4ccccc4c4c(N(c5ccccc5-c5ccccc5)c5cccc6ccccc56)cccc43)cc2-c2ccccc2)cc1 ; c1ccc(-c2ccc(-n3c4ccccc4c4c(N(c5ccc6ccccc6c5)c5ccccc5-c5ccccc5)cccc43)cc2-c2ccccc2)cc1]

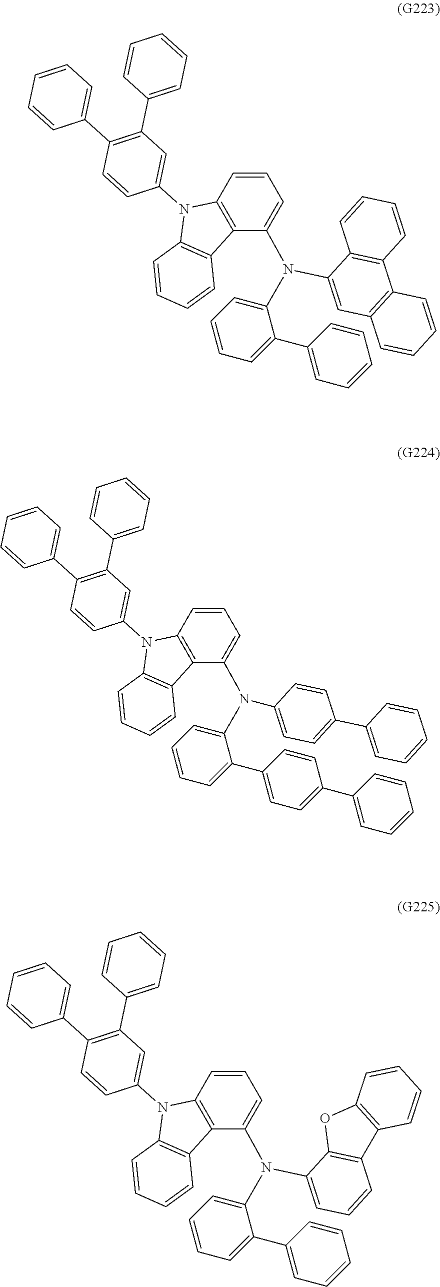 [SMILES: c1ccc(-c2ccc(-n3c4ccccc4c4c(N(c5ccccc5-c5ccccc5)c5cc6ccccc6c6ccccc56)cccc43)cc2-c2ccccc2)cc1 ; c1ccc(-c2ccc(-c3ccccc3N(c3ccc(-c4ccccc4)cc3)c3cccc4c3c3ccccc3n4-c3ccc(-c4ccccc4)c(-c4ccccc4)c3)cc2)cc1 ; c1ccc(-c2ccc(-n3c4ccccc4c4c(N(c5ccccc5-c5ccccc5)c5cccc6c5oc5ccccc56)cccc43)cc2-c2ccccc2)cc1]

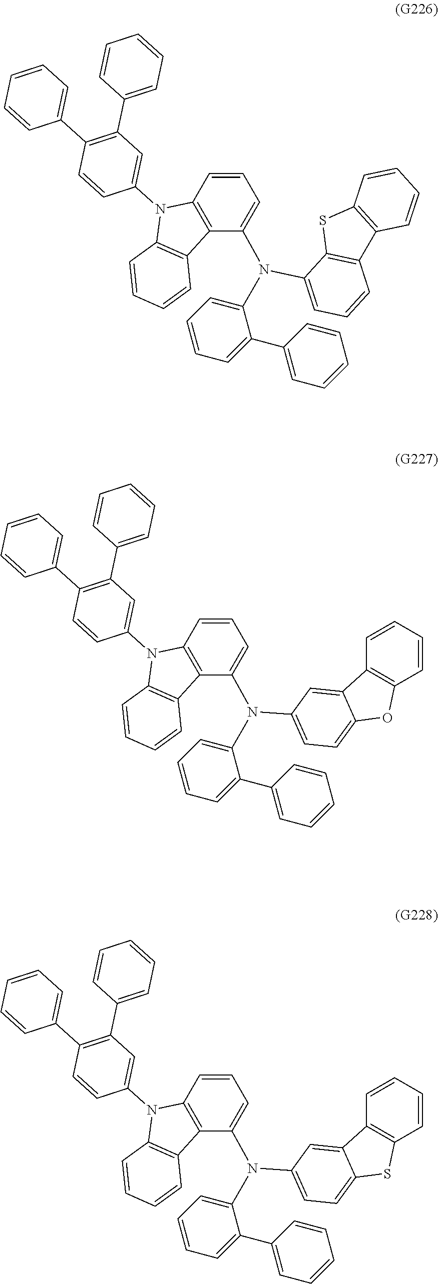 [SMILES: c1ccc(-c2ccc(-n3c4ccccc4c4c(N(c5ccccc5-c5ccccc5)c5cccc6c5sc5ccccc56)cccc43)cc2-c2ccccc2)cc1 ; c1ccc(-c2ccc(-n3c4ccccc4c4c(N(c5ccc6oc7ccccc7c6c5)c5ccccc5-c5ccccc5)cccc43)cc2-c2ccccc2)cc1 ; c1ccc(-c2ccc(-n3c4ccccc4c4c(N(c5ccc6sc7ccccc7c6c5)c5ccccc5-c5ccccc5)cccc43)cc2-c2ccccc2)cc1]

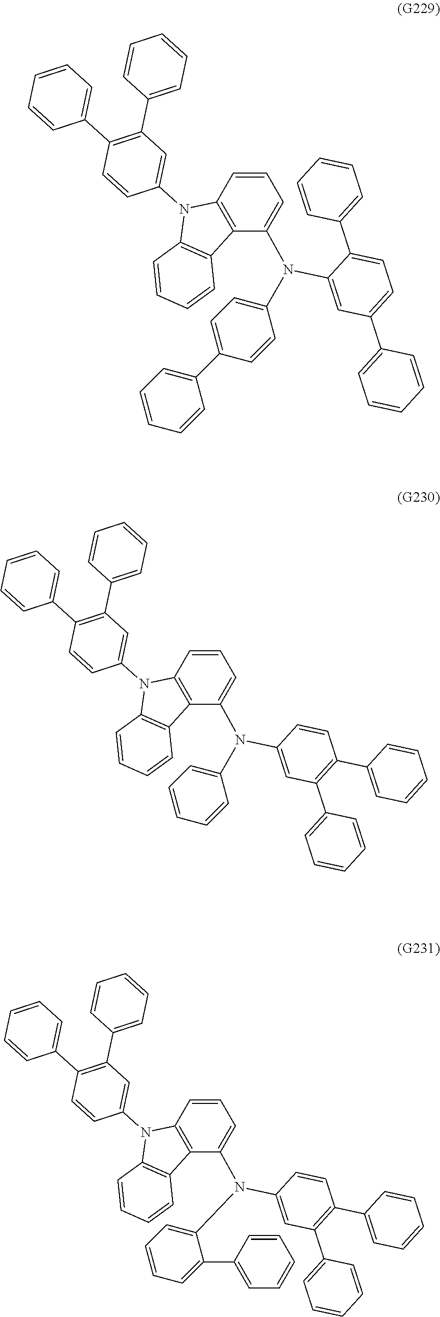 [SMILES: c1ccc(-c2ccc(N(c3cc(-c4ccccc4)ccc3-c3ccccc3)c3cccc4c3c3ccccc3n4-c3ccc(-c4ccccc4)c(-c4ccccc4)c3)cc2)cc1 ; c1ccc(-c2ccc(N(c3ccccc3)c3cccc4c3c3ccccc3n4-c3ccc(-c4ccccc4)c(-c4ccccc4)c3)cc2-c2ccccc2)cc1 ; c1ccc(-c2ccc(N(c3ccccc3-c3ccccc3)c3cccc4c3c3ccccc3n4-c3ccc(-c4ccccc4)c(-c4ccccc4)c3)cc2-c2ccccc2)cc1]

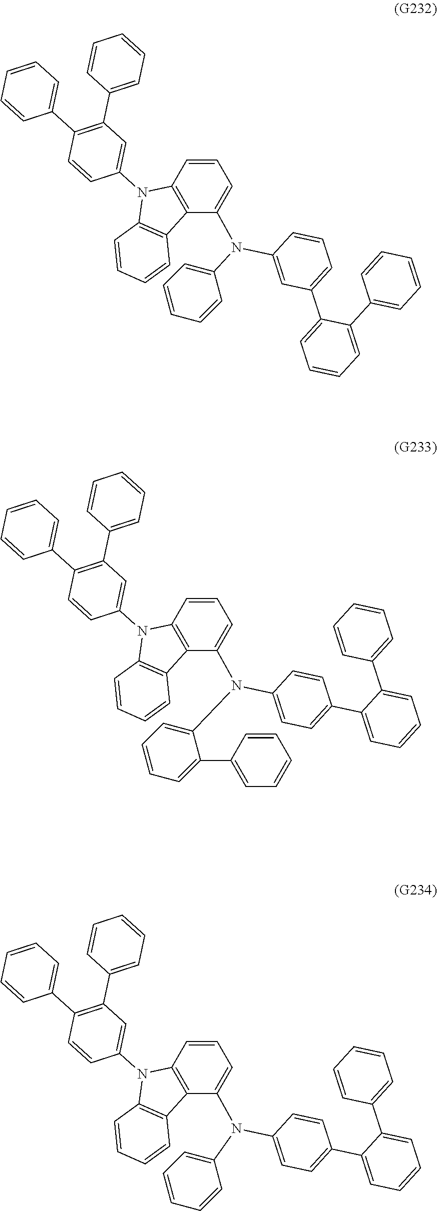 [SMILES: c1ccc(-c2ccccc2-c2cccc(N(c3ccccc3)c3cccc4c3c3ccccc3n4-c3ccc(-c4ccccc4)c(-c4ccccc4)c3)c2)cc1 ; c1ccc(-c2ccccc2-c2ccc(N(c3ccccc3-c3ccccc3)c3cccc4c3c3ccccc3n4-c3ccc(-c4ccccc4)c(-c4ccccc4)c3)cc2)cc1 ; c1ccc(-c2ccccc2-c2ccc(N(c3ccccc3)c3cccc4c3c3ccccc3n4-c3ccc(-c4ccccc4)c(-c4ccccc4)c3)cc2)cc1]

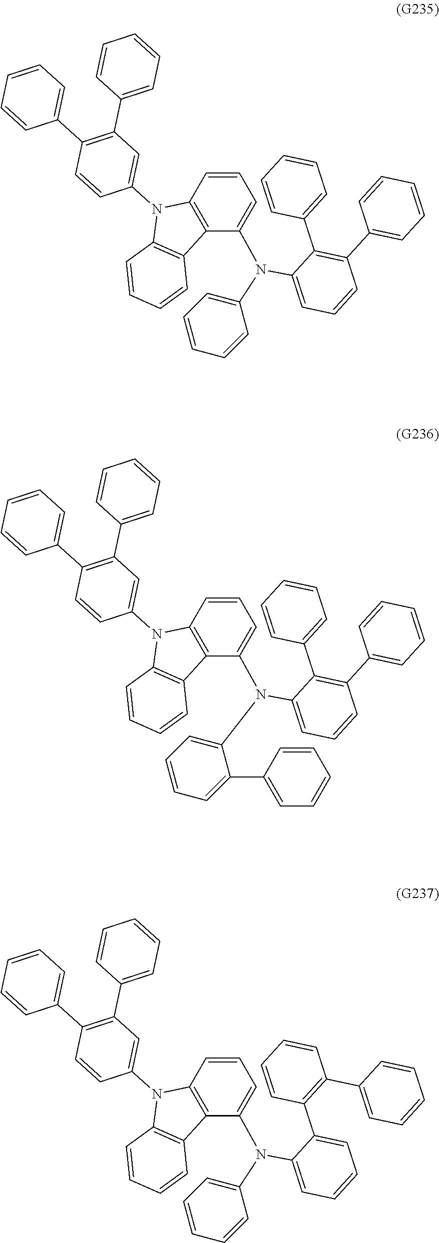 [SMILES: c1ccc(-c2ccc(-n3c4ccccc4c4c(N(c5ccccc5)c5cccc(-c6ccccc6)c5-c5ccccc5)cccc43)cc2-c2ccccc2)cc1 ; c1ccc(-c2ccc(-n3c4ccccc4c4c(N(c5ccccc5-c5ccccc5)c5cccc(-c6ccccc6)c5-c5ccccc5)cccc43)cc2-c2ccccc2)cc1 ; c1ccc(-c2ccc(-n3c4ccccc4c4c(N(c5ccccc5)c5ccccc5-c5ccccc5-c5ccccc5)cccc43)cc2-c2ccccc2)cc1]

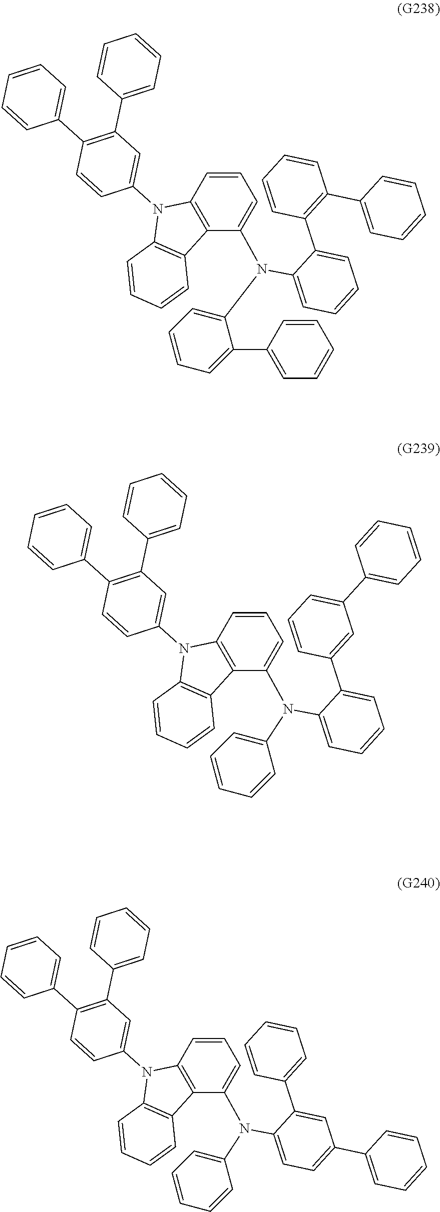 [SMILES: c1ccc(-c2ccc(-n3c4ccccc4c4c(N(c5ccccc5-c5ccccc5)c5ccccc5-c5ccccc5-c5ccccc5)cccc43)cc2-c2ccccc2)cc1 ; c1ccc(-c2cccc(-c3ccccc3N(c3ccccc3)c3cccc4c3c3ccccc3n4-c3ccc(-c4ccccc4)c(-c4ccccc4)c3)c2)cc1 ; c1ccc(-c2ccc(N(c3ccccc3)c3cccc4c3c3ccccc3n4-c3ccc(-c4ccccc4)c(-c4ccccc4)c3)c(-c3ccccc3)c2)cc1]

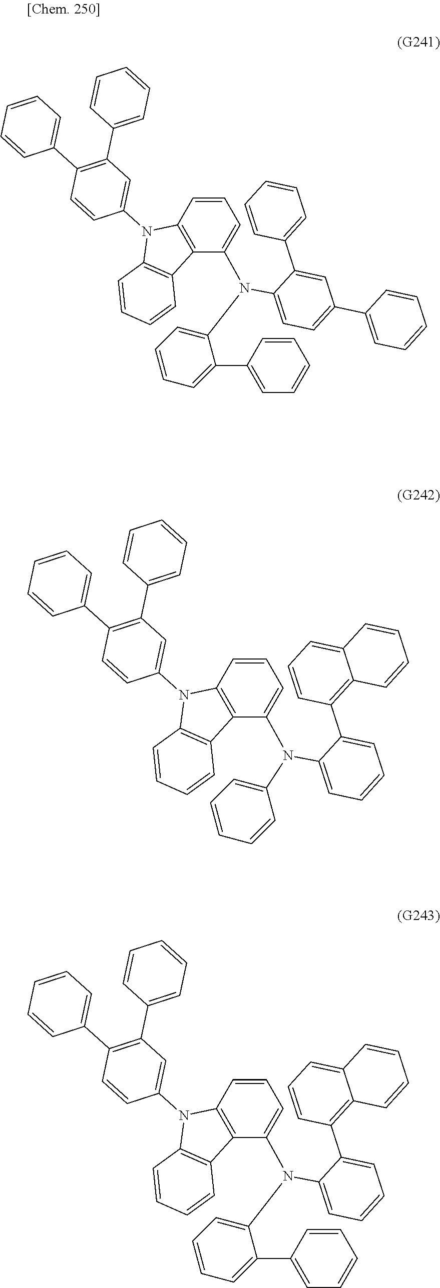 [SMILES: c1ccc(-c2ccc(N(c3ccccc3-c3ccccc3)c3cccc4c3c3ccccc3n4-c3ccc(-c4ccccc4)c(-c4ccccc4)c3)c(-c3ccccc3)c2)cc1 ; c1ccc(-c2ccc(-n3c4ccccc4c4c(N(c5ccccc5)c5ccccc5-c5cccc6ccccc56)cccc43)cc2-c2ccccc2)cc1 ; c1ccc(-c2ccc(-n3c4ccccc4c4c(N(c5ccccc5-c5ccccc5)c5ccccc5-c5cccc6ccccc56)cccc43)cc2-c2ccccc2)cc1]

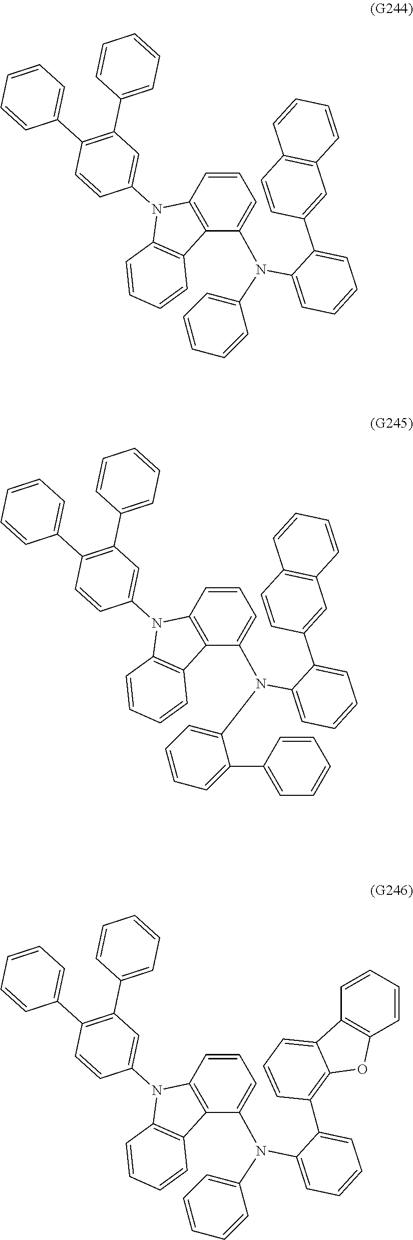 [SMILES: c1ccc(-c2ccc(-n3c4ccccc4c4c(N(c5ccccc5)c5ccccc5-c5ccc6ccccc6c5)cccc43)cc2-c2ccccc2)cc1 ; c1ccc(-c2ccc(-n3c4ccccc4c4c(N(c5ccccc5-c5ccccc5)c5ccccc5-c5ccc6ccccc6c5)cccc43)cc2-c2ccccc2)cc1 ; c1ccc(-c2ccc(-n3c4ccccc4c4c(N(c5ccccc5)c5ccccc5-c5cccc6c5oc5ccccc56)cccc43)cc2-c2ccccc2)cc1]

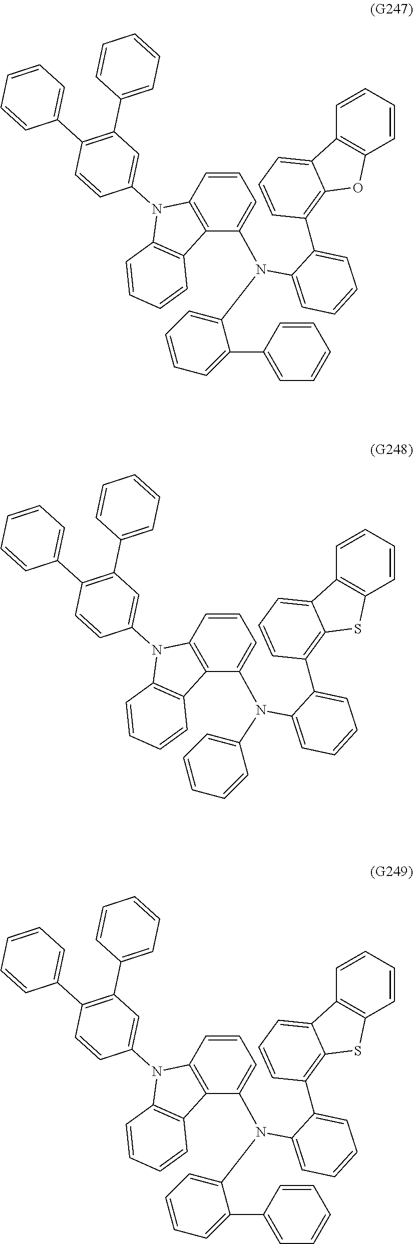 [SMILES: c1ccc(-c2ccc(-n3c4ccccc4c4c(N(c5ccccc5-c5ccccc5)c5ccccc5-c5cccc6c5oc5ccccc56)cccc43)cc2-c2ccccc2)cc1 ; c1ccc(-c2ccc(-n3c4ccccc4c4c(N(c5ccccc5)c5ccccc5-c5cccc6c5sc5ccccc56)cccc43)cc2-c2ccccc2)cc1 ; c1ccc(-c2ccc(-n3c4ccccc4c4c(N(c5ccccc5-c5ccccc5)c5ccccc5-c5cccc6c5sc5ccccc56)cccc43)cc2-c2ccccc2)cc1]

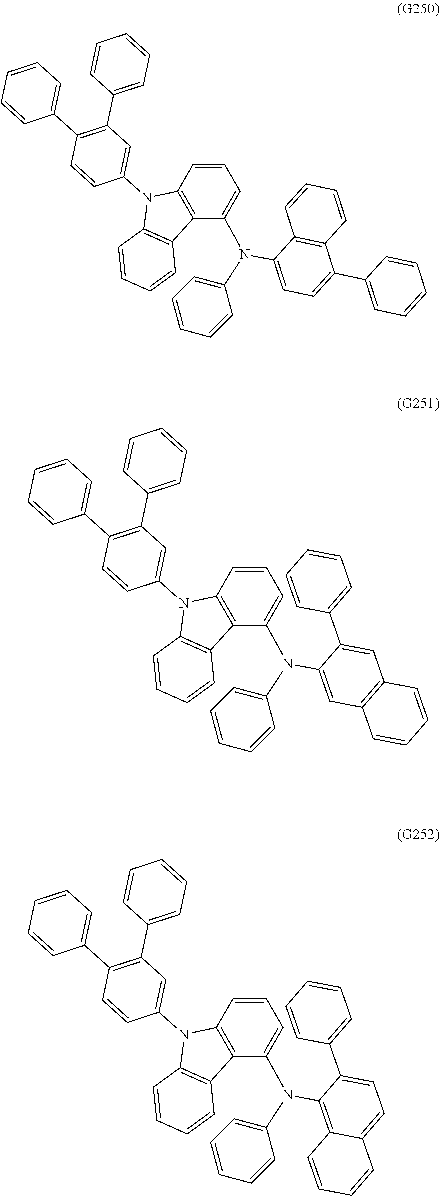 [SMILES: c1ccc(-c2ccc(-n3c4ccccc4c4c(N(c5ccccc5)c5ccc(-c6ccccc6)c6ccccc56)cccc43)cc2-c2ccccc2)cc1 ; c1ccc(-c2ccc(-n3c4ccccc4c4c(N(c5ccccc5)c5cc6ccccc6cc5-c5ccccc5)cccc43)cc2-c2ccccc2)cc1 ; c1ccc(-c2ccc(-n3c4ccccc4c4c(N(c5ccccc5)c5c(-c6ccccc6)ccc6ccccc56)cccc43)cc2-c2ccccc2)cc1]

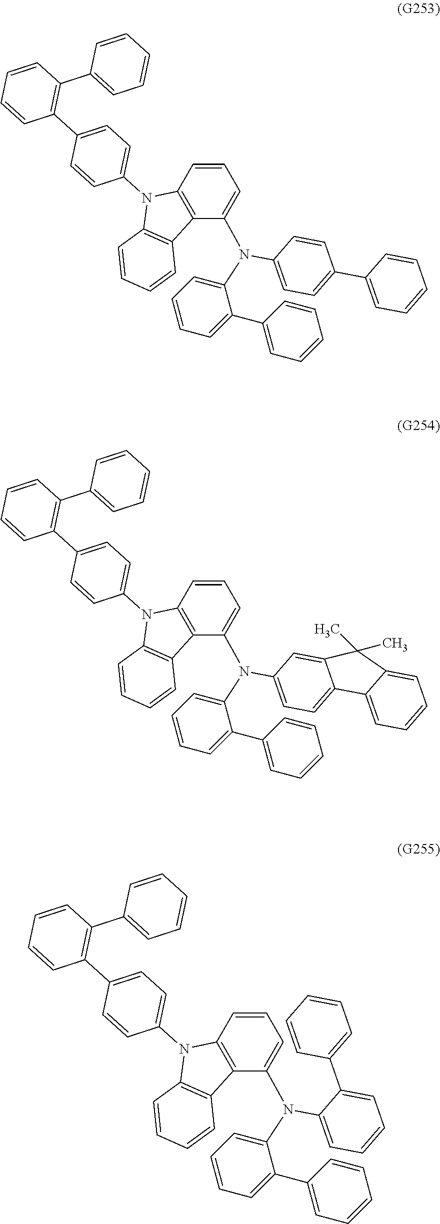 [SMILES: c1ccc(-c2ccc(N(c3ccccc3-c3ccccc3)c3cccc4c3c3ccccc3n4-c3ccc(-c4ccccc4-c4ccccc4)cc3)cc2)cc1 ; CC1(C)c2ccccc2-c2ccc(N(c3ccccc3-c3ccccc3)c3cccc4c3c3ccccc3n4-c3ccc(-c4ccccc4-c4ccccc4)cc3)cc21 ; c1ccc(-c2ccccc2-c2ccc(-n3c4ccccc4c4c(N(c5ccccc5-c5ccccc5)c5ccccc5-c5ccccc5)cccc43)cc2)cc1]

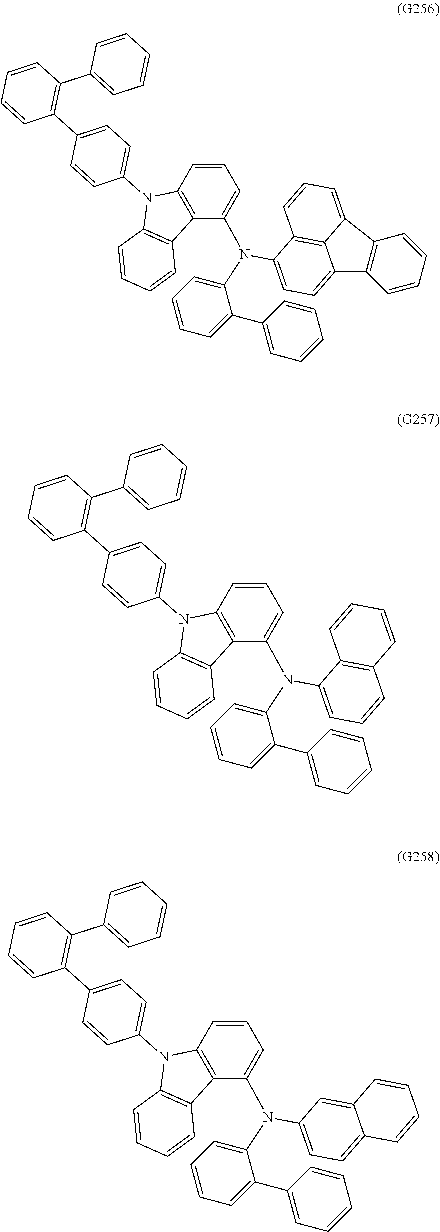 [SMILES: c1ccc(-c2ccccc2-c2ccc(-n3c4ccccc4c4c(N(c5ccccc5-c5ccccc5)c5ccc6c7c(cccc57)-c5ccccc5-6)cccc43)cc2)cc1 ; c1ccc(-c2ccccc2-c2ccc(-n3c4ccccc4c4c(N(c5ccccc5-c5ccccc5)c5cccc6ccccc56)cccc43)cc2)cc1 ; c1ccc(-c2ccccc2-c2ccc(-n3c4ccccc4c4c(N(c5ccc6ccccc6c5)c5ccccc5-c5ccccc5)cccc43)cc2)cc1]

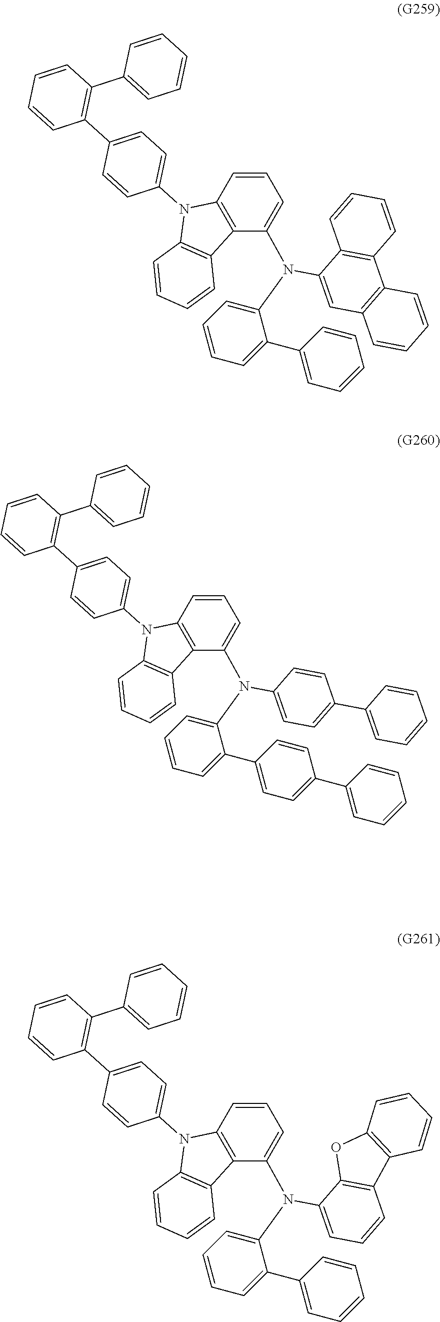 [SMILES: c1ccc(-c2ccccc2-c2ccc(-n3c4ccccc4c4c(N(c5ccccc5-c5ccccc5)c5cc6ccccc6c6ccccc56)cccc43)cc2)cc1 ; c1ccc(-c2ccc(-c3ccccc3N(c3ccc(-c4ccccc4)cc3)c3cccc4c3c3ccccc3n4-c3ccc(-c4ccccc4-c4ccccc4)cc3)cc2)cc1 ; c1ccc(-c2ccccc2-c2ccc(-n3c4ccccc4c4c(N(c5ccccc5-c5ccccc5)c5cccc6c5oc5ccccc56)cccc43)cc2)cc1]

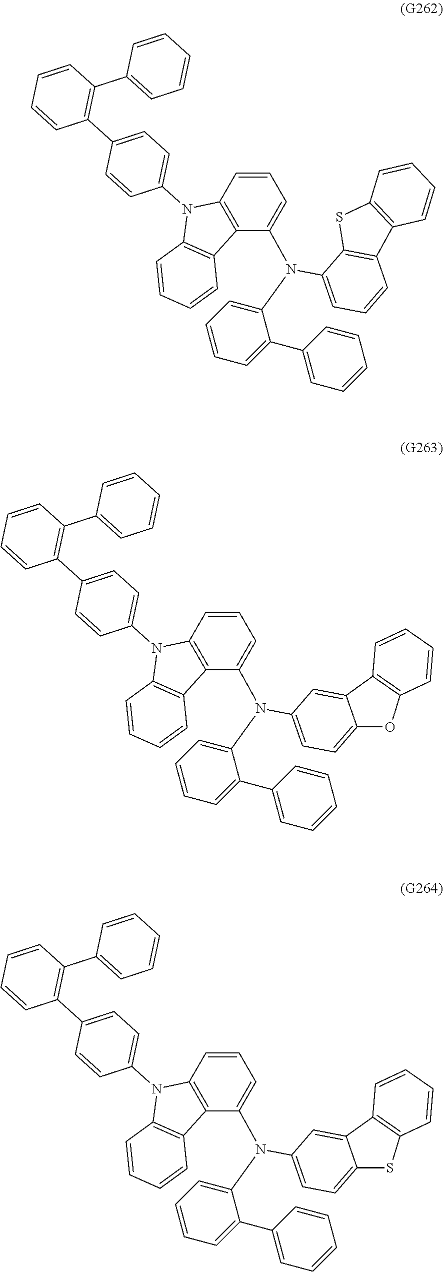 [SMILES: c1ccc(-c2ccccc2-c2ccc(-n3c4ccccc4c4c(N(c5ccccc5-c5ccccc5)c5cccc6c5sc5ccccc56)cccc43)cc2)cc1 ; c1ccc(-c2ccccc2-c2ccc(-n3c4ccccc4c4c(N(c5ccc6oc7ccccc7c6c5)c5ccccc5-c5ccccc5)cccc43)cc2)cc1 ; c1ccc(-c2ccccc2-c2ccc(-n3c4ccccc4c4c(N(c5ccc6sc7ccccc7c6c5)c5ccccc5-c5ccccc5)cccc43)cc2)cc1]

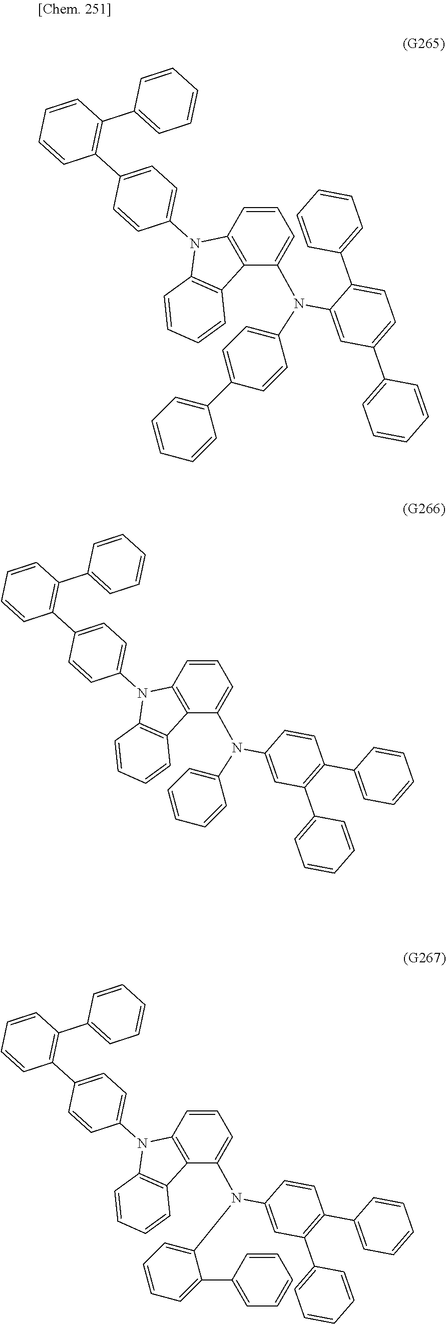 [SMILES: c1ccc(-c2ccc(N(c3cc(-c4ccccc4)ccc3-c3ccccc3)c3cccc4c3c3ccccc3n4-c3ccc(-c4ccccc4-c4ccccc4)cc3)cc2)cc1 ; c1ccc(-c2ccccc2-c2ccc(-n3c4ccccc4c4c(N(c5ccccc5)c5ccc(-c6ccccc6)c(-c6ccccc6)c5)cccc43)cc2)cc1 ; c1ccc(-c2ccccc2-c2ccc(-n3c4ccccc4c4c(N(c5ccc(-c6ccccc6)c(-c6ccccc6)c5)c5ccccc5-c5ccccc5)cccc43)cc2)cc1]

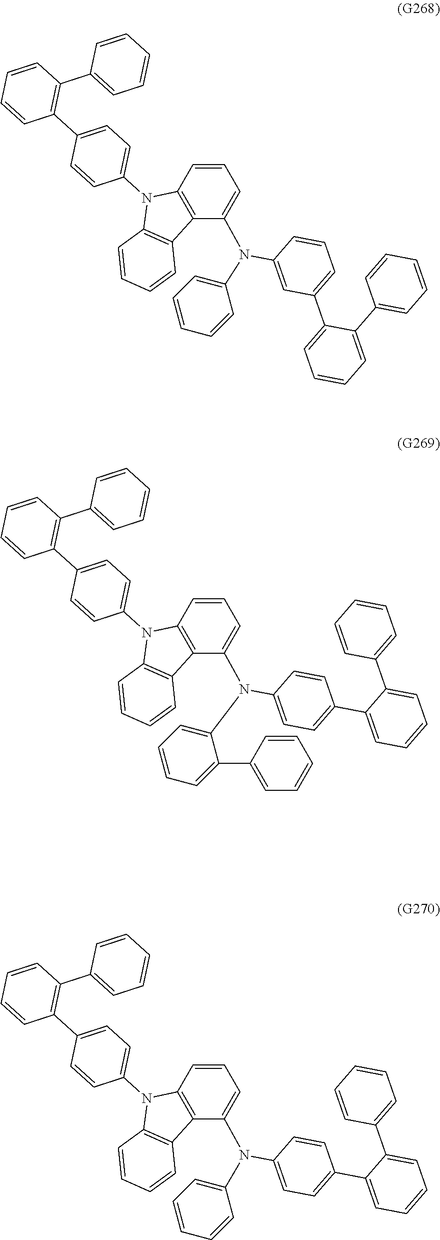 [SMILES: c1ccc(-c2ccccc2-c2ccc(-n3c4ccccc4c4c(N(c5ccccc5)c5cccc(-c6ccccc6-c6ccccc6)c5)cccc43)cc2)cc1 ; c1ccc(-c2ccccc2-c2ccc(N(c3ccccc3-c3ccccc3)c3cccc4c3c3ccccc3n4-c3ccc(-c4ccccc4-c4ccccc4)cc3)cc2)cc1 ; c1ccc(-c2ccccc2-c2ccc(N(c3ccccc3)c3cccc4c3c3ccccc3n4-c3ccc(-c4ccccc4-c4ccccc4)cc3)cc2)cc1]

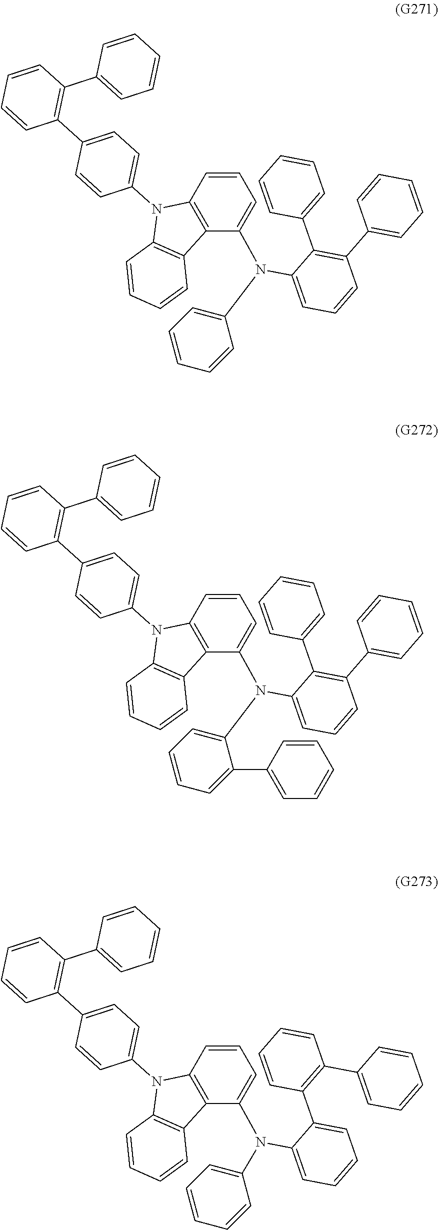 [SMILES: c1ccc(-c2ccccc2-c2ccc(-n3c4ccccc4c4c(N(c5ccccc5)c5cccc(-c6ccccc6)c5-c5ccccc5)cccc43)cc2)cc1 ; c1ccc(-c2ccccc2-c2ccc(-n3c4ccccc4c4c(N(c5ccccc5-c5ccccc5)c5cccc(-c6ccccc6)c5-c5ccccc5)cccc43)cc2)cc1 ; c1ccc(-c2ccccc2-c2ccc(-n3c4ccccc4c4c(N(c5ccccc5)c5ccccc5-c5ccccc5-c5ccccc5)cccc43)cc2)cc1]

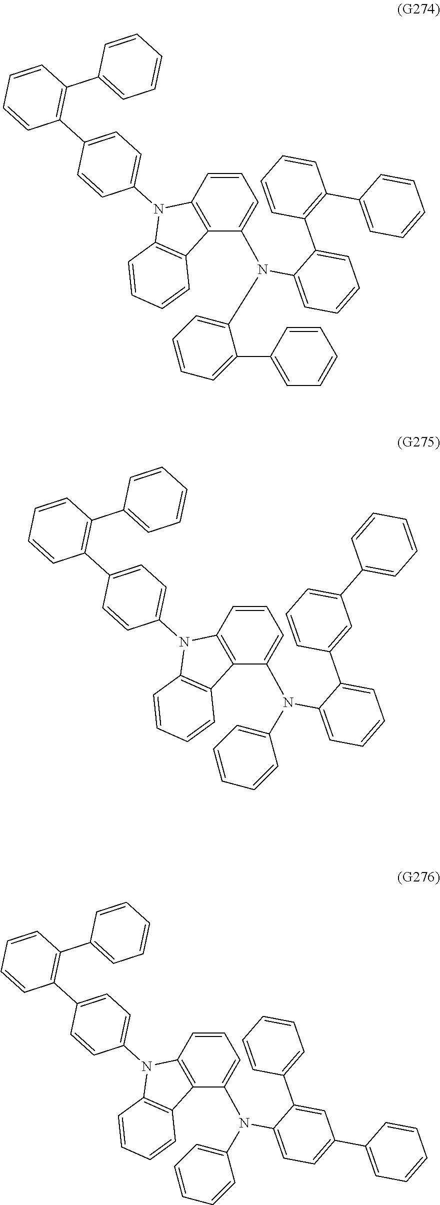 [SMILES: c1ccc(-c2ccccc2-c2ccc(-n3c4ccccc4c4c(N(c5ccccc5-c5ccccc5)c5ccccc5-c5ccccc5-c5ccccc5)cccc43)cc2)cc1 ; c1ccc(-c2cccc(-c3ccccc3N(c3ccccc3)c3cccc4c3c3ccccc3n4-c3ccc(-c4ccccc4-c4ccccc4)cc3)c2)cc1 ; c1ccc(-c2ccc(N(c3ccccc3)c3cccc4c3c3ccccc3n4-c3ccc(-c4ccccc4-c4ccccc4)cc3)c(-c3ccccc3)c2)cc1]

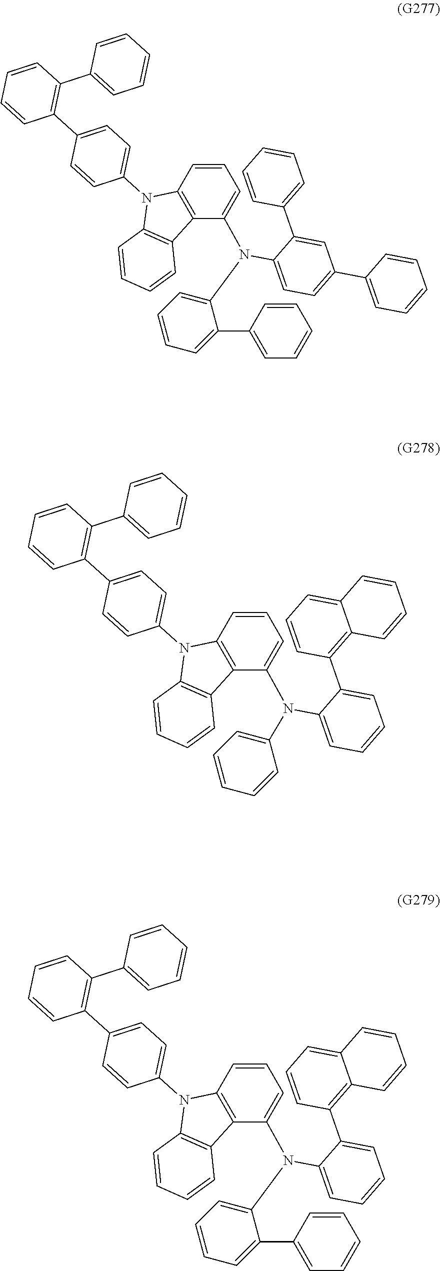 [SMILES: c1ccc(-c2ccc(N(c3ccccc3-c3ccccc3)c3cccc4c3c3ccccc3n4-c3ccc(-c4ccccc4-c4ccccc4)cc3)c(-c3ccccc3)c2)cc1 ; c1ccc(-c2ccccc2-c2ccc(-n3c4ccccc4c4c(N(c5ccccc5)c5ccccc5-c5cccc6ccccc56)cccc43)cc2)cc1 ; c1ccc(-c2ccccc2-c2ccc(-n3c4ccccc4c4c(N(c5ccccc5-c5ccccc5)c5ccccc5-c5cccc6ccccc56)cccc43)cc2)cc1]

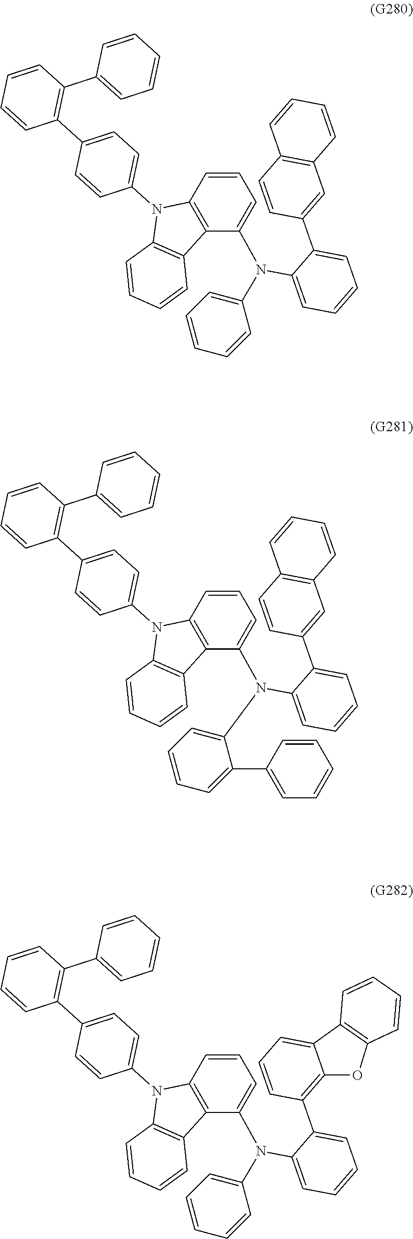 [SMILES: c1ccc(-c2ccccc2-c2ccc(-n3c4ccccc4c4c(N(c5ccccc5)c5ccccc5-c5ccc6ccccc6c5)cccc43)cc2)cc1 ; c1ccc(-c2ccccc2-c2ccc(-n3c4ccccc4c4c(N(c5ccccc5-c5ccccc5)c5ccccc5-c5ccc6ccccc6c5)cccc43)cc2)cc1 ; c1ccc(-c2ccccc2-c2ccc(-n3c4ccccc4c4c(N(c5ccccc5)c5ccccc5-c5cccc6c5oc5ccccc56)cccc43)cc2)cc1]

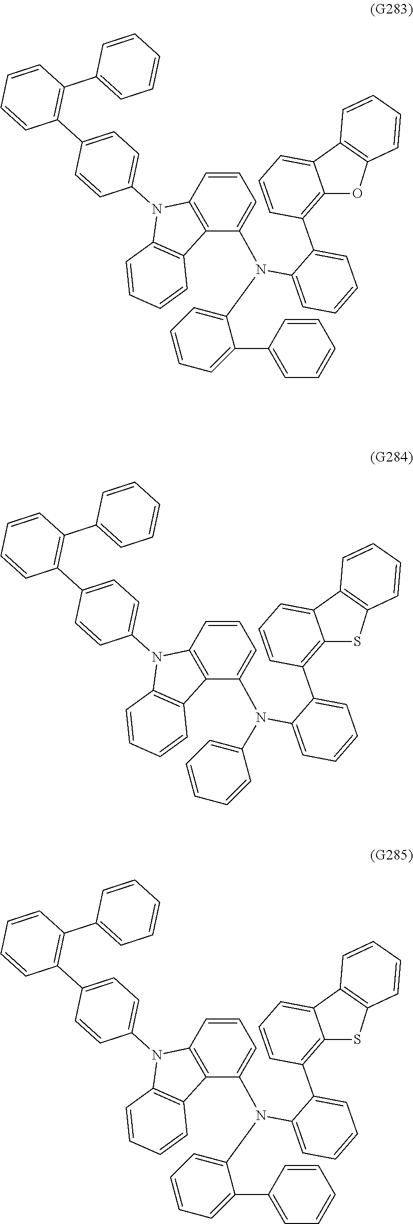 [SMILES: c1ccc(-c2ccccc2-c2ccc(-n3c4ccccc4c4c(N(c5ccccc5-c5ccccc5)c5ccccc5-c5cccc6c5oc5ccccc56)cccc43)cc2)cc1 ; c1ccc(-c2ccccc2-c2ccc(-n3c4ccccc4c4c(N(c5ccccc5)c5ccccc5-c5cccc6c5sc5ccccc56)cccc43)cc2)cc1 ; c1ccc(-c2ccccc2-c2ccc(-n3c4ccccc4c4c(N(c5ccccc5-c5ccccc5)c5ccccc5-c5cccc6c5sc5ccccc56)cccc43)cc2)cc1]

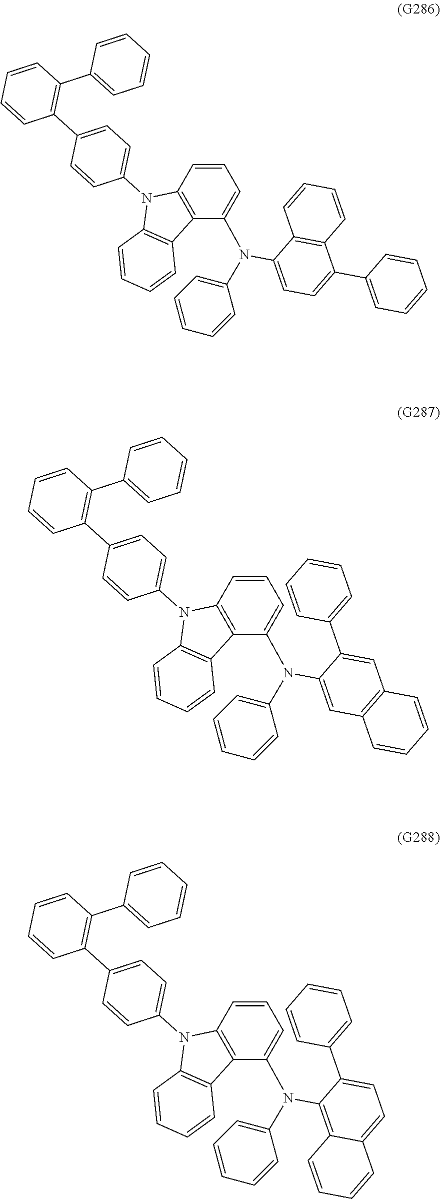 [SMILES: c1ccc(-c2ccccc2-c2ccc(-n3c4ccccc4c4c(N(c5ccccc5)c5ccc(-c6ccccc6)c6ccccc56)cccc43)cc2)cc1 ; c1ccc(-c2ccccc2-c2ccc(-n3c4ccccc4c4c(N(c5ccccc5)c5cc6ccccc6cc5-c5ccccc5)cccc43)cc2)cc1 ; c1ccc(-c2ccccc2-c2ccc(-n3c4ccccc4c4c(N(c5ccccc5)c5c(-c6ccccc6)ccc6ccccc56)cccc43)cc2)cc1]

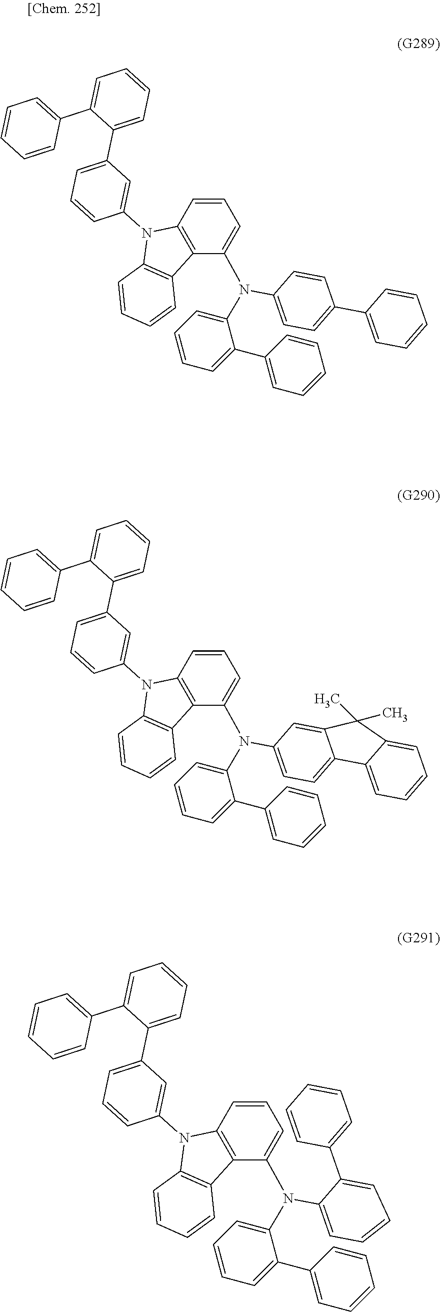 [SMILES: c1ccc(-c2ccc(N(c3ccccc3-c3ccccc3)c3cccc4c3c3ccccc3n4-c3cccc(-c4ccccc4-c4ccccc4)c3)cc2)cc1 ; CC1(C)c2ccccc2-c2ccc(N(c3ccccc3-c3ccccc3)c3cccc4c3c3ccccc3n4-c3cccc(-c4ccccc4-c4ccccc4)c3)cc21 ; c1ccc(-c2ccccc2-c2cccc(-n3c4ccccc4c4c(N(c5ccccc5-c5ccccc5)c5ccccc5-c5ccccc5)cccc43)c2)cc1]

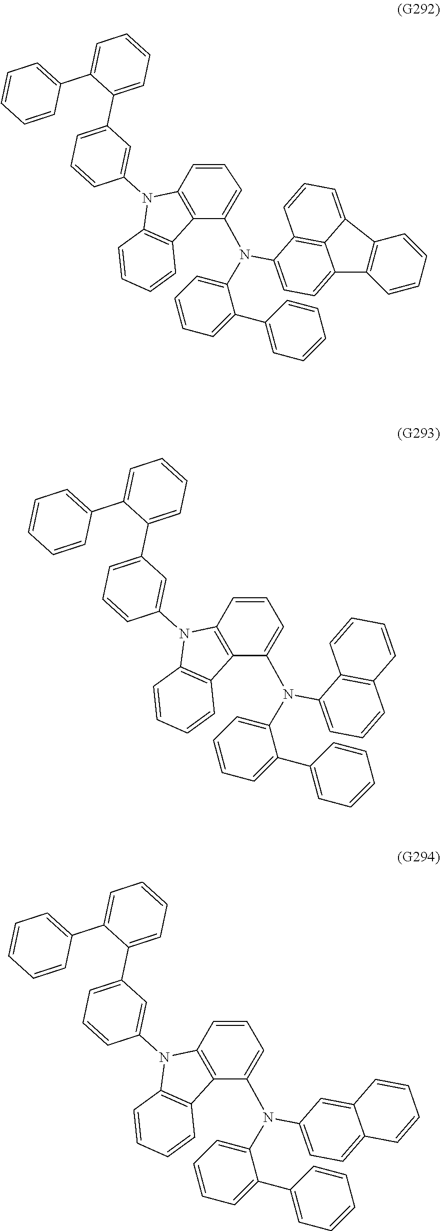 [SMILES: c1ccc(-c2ccccc2-c2cccc(-n3c4ccccc4c4c(N(c5ccccc5-c5ccccc5)c5ccc6c7c(cccc57)-c5ccccc5-6)cccc43)c2)cc1 ; c1ccc(-c2ccccc2-c2cccc(-n3c4ccccc4c4c(N(c5ccccc5-c5ccccc5)c5cccc6ccccc56)cccc43)c2)cc1 ; c1ccc(-c2ccccc2-c2cccc(-n3c4ccccc4c4c(N(c5ccc6ccccc6c5)c5ccccc5-c5ccccc5)cccc43)c2)cc1]

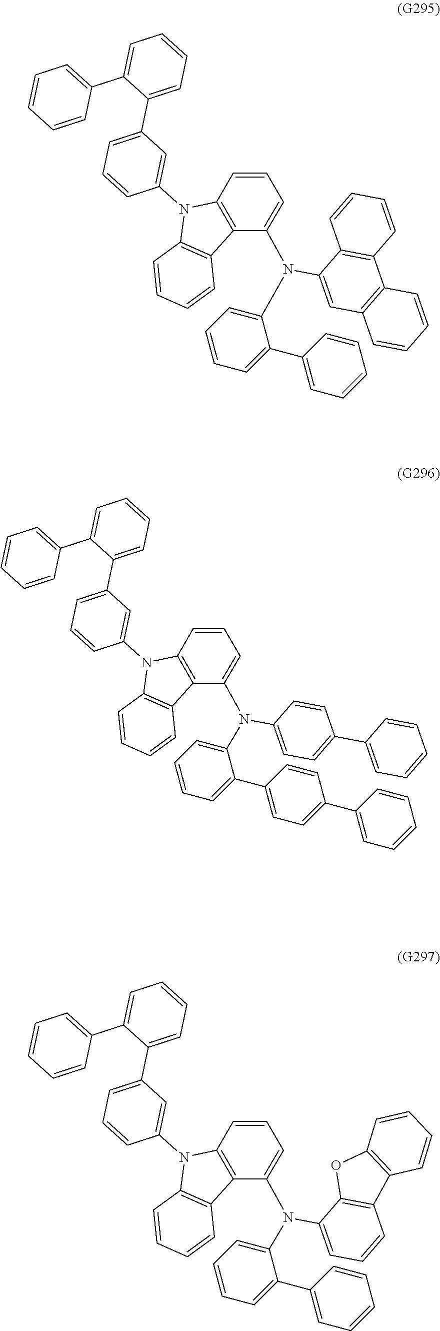 [SMILES: c1ccc(-c2ccccc2-c2cccc(-n3c4ccccc4c4c(N(c5ccccc5-c5ccccc5)c5cc6ccccc6c6ccccc56)cccc43)c2)cc1 ; c1ccc(-c2ccc(-c3ccccc3N(c3ccc(-c4ccccc4)cc3)c3cccc4c3c3ccccc3n4-c3cccc(-c4ccccc4-c4ccccc4)c3)cc2)cc1 ; c1ccc(-c2ccccc2-c2cccc(-n3c4ccccc4c4c(N(c5ccccc5-c5ccccc5)c5cccc6c5oc5ccccc56)cccc43)c2)cc1]

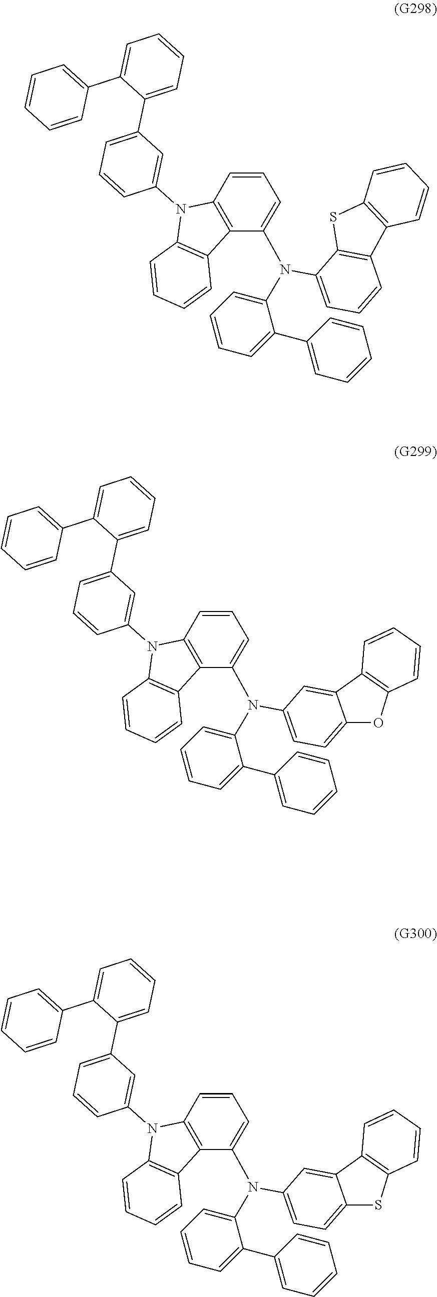 [SMILES: c1ccc(-c2ccccc2-c2cccc(-n3c4ccccc4c4c(N(c5ccccc5-c5ccccc5)c5cccc6c5sc5ccccc56)cccc43)c2)cc1 ; c1ccc(-c2ccccc2-c2cccc(-n3c4ccccc4c4c(N(c5ccc6oc7ccccc7c6c5)c5ccccc5-c5ccccc5)cccc43)c2)cc1 ; c1ccc(-c2ccccc2-c2cccc(-n3c4ccccc4c4c(N(c5ccc6sc7ccccc7c6c5)c5ccccc5-c5ccccc5)cccc43)c2)cc1]

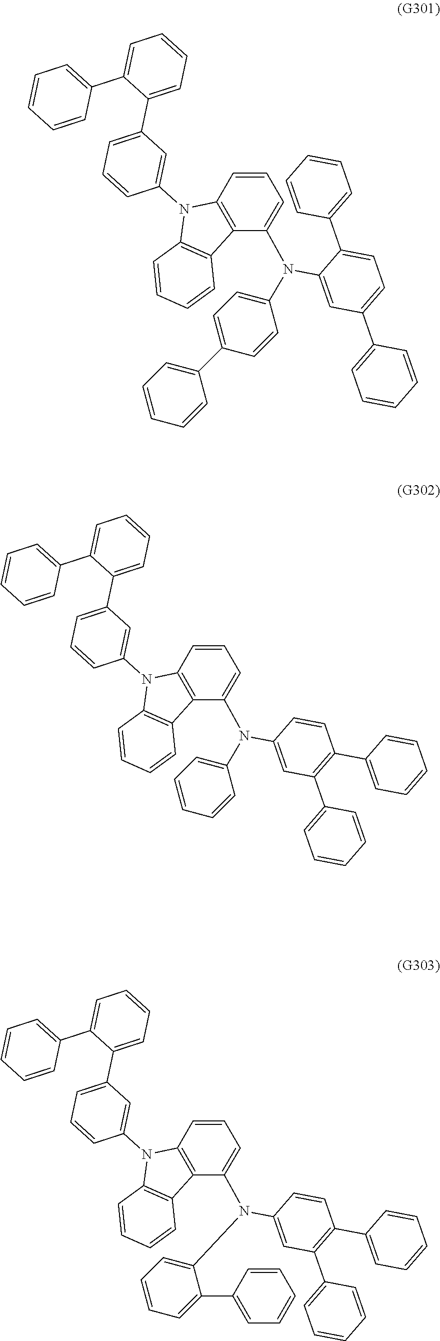 [SMILES: c1ccc(-c2ccc(N(c3cc(-c4ccccc4)ccc3-c3ccccc3)c3cccc4c3c3ccccc3n4-c3cccc(-c4ccccc4-c4ccccc4)c3)cc2)cc1 ; c1ccc(-c2ccccc2-c2cccc(-n3c4ccccc4c4c(N(c5ccccc5)c5ccc(-c6ccccc6)c(-c6ccccc6)c5)cccc43)c2)cc1 ; c1ccc(-c2ccccc2-c2cccc(-n3c4ccccc4c4c(N(c5ccc(-c6ccccc6)c(-c6ccccc6)c5)c5ccccc5-c5ccccc5)cccc43)c2)cc1]

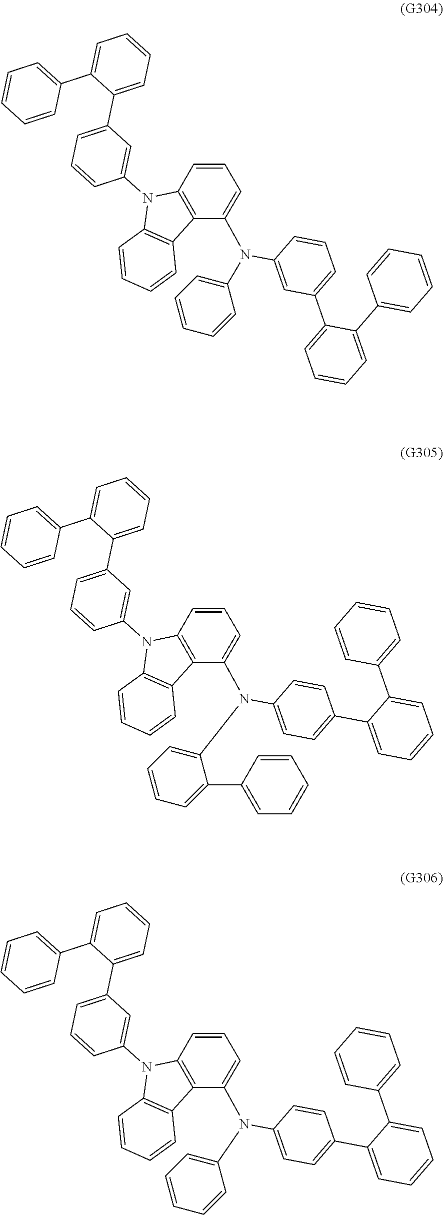 [SMILES: c1ccc(-c2ccccc2-c2cccc(N(c3ccccc3)c3cccc4c3c3ccccc3n4-c3cccc(-c4ccccc4-c4ccccc4)c3)c2)cc1 ; c1ccc(-c2ccccc2-c2ccc(N(c3ccccc3-c3ccccc3)c3cccc4c3c3ccccc3n4-c3cccc(-c4ccccc4-c4ccccc4)c3)cc2)cc1 ; c1ccc(-c2ccccc2-c2ccc(N(c3ccccc3)c3cccc4c3c3ccccc3n4-c3cccc(-c4ccccc4-c4ccccc4)c3)cc2)cc1]

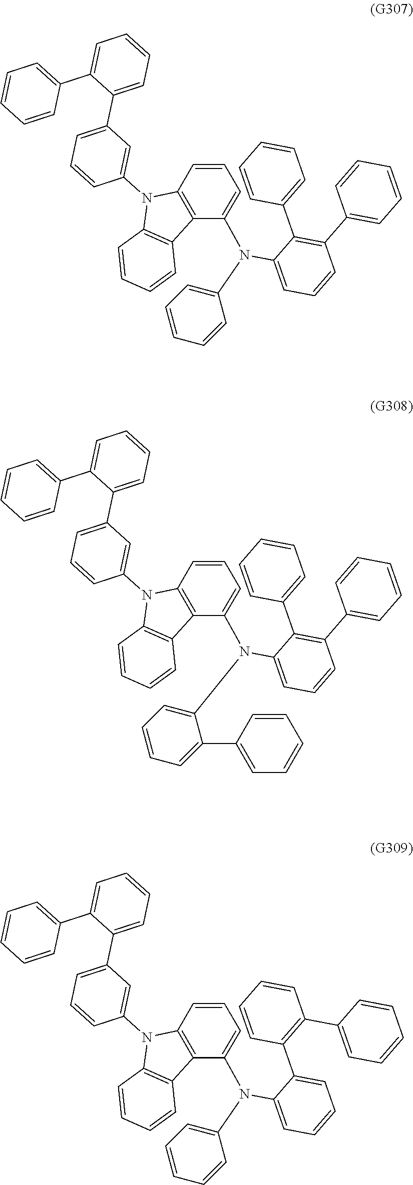 [SMILES: c1ccc(-c2ccccc2-c2cccc(-n3c4ccccc4c4c(N(c5ccccc5)c5cccc(-c6ccccc6)c5-c5ccccc5)cccc43)c2)cc1 ; c1ccc(-c2ccccc2-c2cccc(-n3c4ccccc4c4c(N(c5ccccc5-c5ccccc5)c5cccc(-c6ccccc6)c5-c5ccccc5)cccc43)c2)cc1 ; c1ccc(-c2ccccc2-c2cccc(-n3c4ccccc4c4c(N(c5ccccc5)c5ccccc5-c5ccccc5-c5ccccc5)cccc43)c2)cc1]

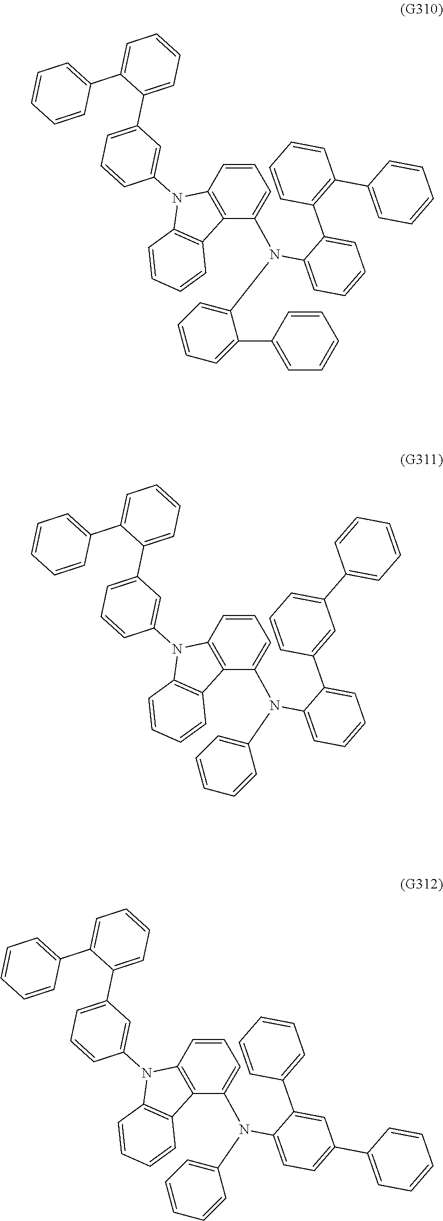 [SMILES: c1ccc(-c2ccccc2-c2cccc(-n3c4ccccc4c4c(N(c5ccccc5-c5ccccc5)c5ccccc5-c5ccccc5-c5ccccc5)cccc43)c2)cc1 ; c1ccc(-c2cccc(-c3ccccc3N(c3ccccc3)c3cccc4c3c3ccccc3n4-c3cccc(-c4ccccc4-c4ccccc4)c3)c2)cc1 ; c1ccc(-c2ccc(N(c3ccccc3)c3cccc4c3c3ccccc3n4-c3cccc(-c4ccccc4-c4ccccc4)c3)c(-c3ccccc3)c2)cc1]

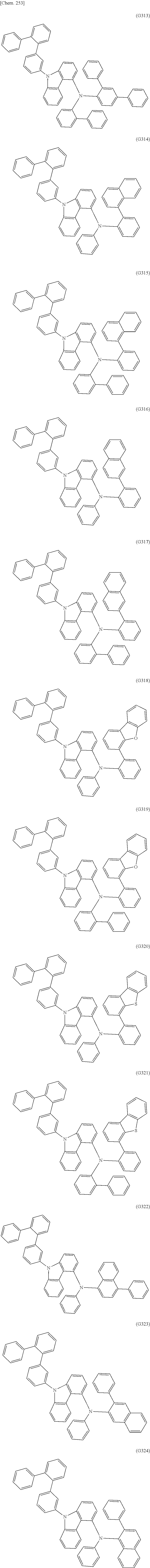

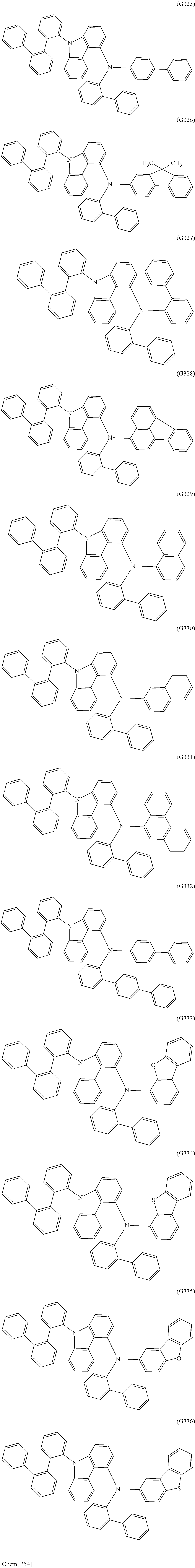

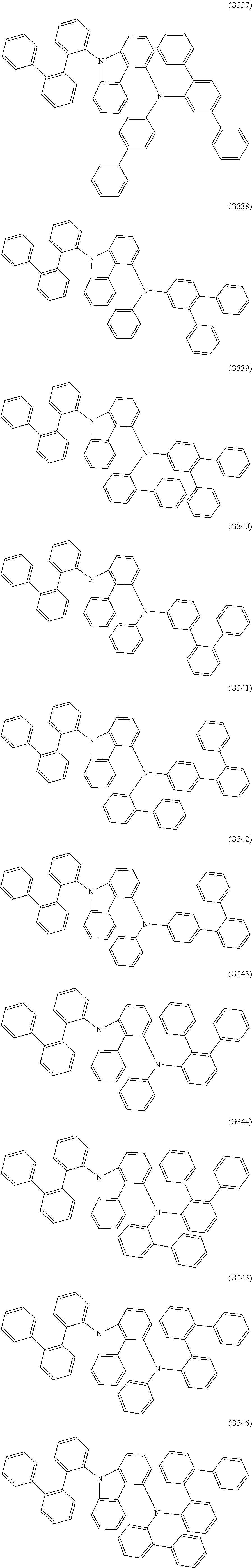

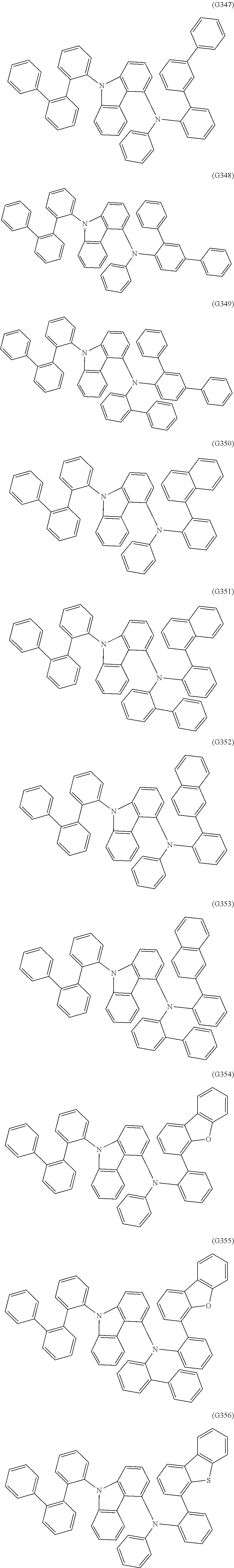

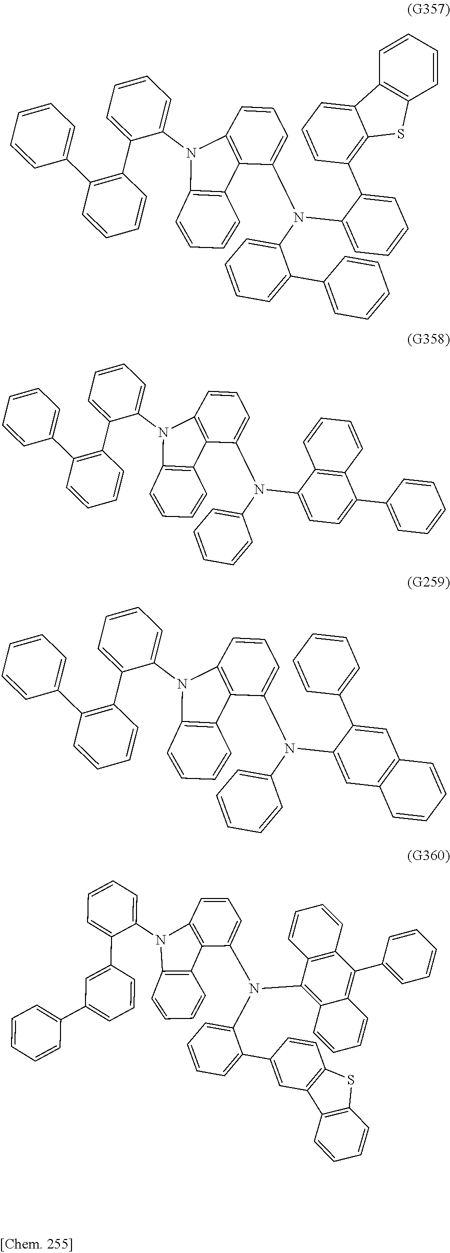 [SMILES: c1ccc(-c2ccccc2-c2ccccc2-n2c3ccccc3c3c(N(c4ccccc4-c4ccccc4)c4ccccc4-c4cccc5c4sc4ccccc45)cccc32)cc1 ; c1ccc(-c2ccccc2-c2ccccc2-n2c3ccccc3c3c(N(c4ccccc4)c4ccc(-c5ccccc5)c5ccccc45)cccc32)cc1 ; c1ccc(-c2ccccc2-c2ccccc2-n2c3ccccc3c3c(N(c4ccccc4)c4cc5ccccc5cc4-c4ccccc4)cccc32)cc1 ; c1ccc(-c2cccc(-c3ccccc3-n3c4ccccc4c4c(N(c5ccccc5-c5ccc6sc7ccccc7c6c5)c5c6ccccc6c(-c6ccccc6)c6ccccc56)cccc43)c2)cc1]

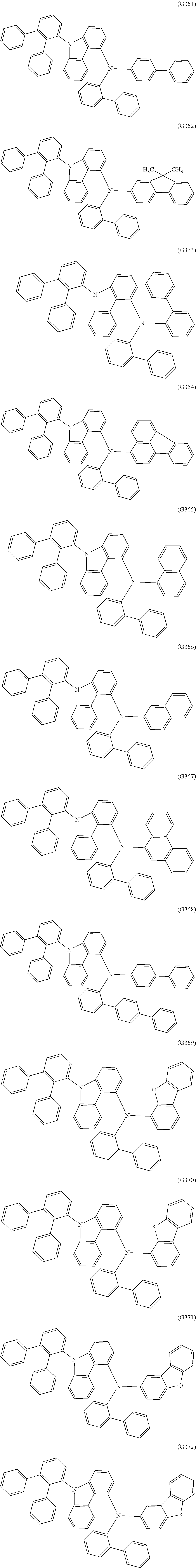

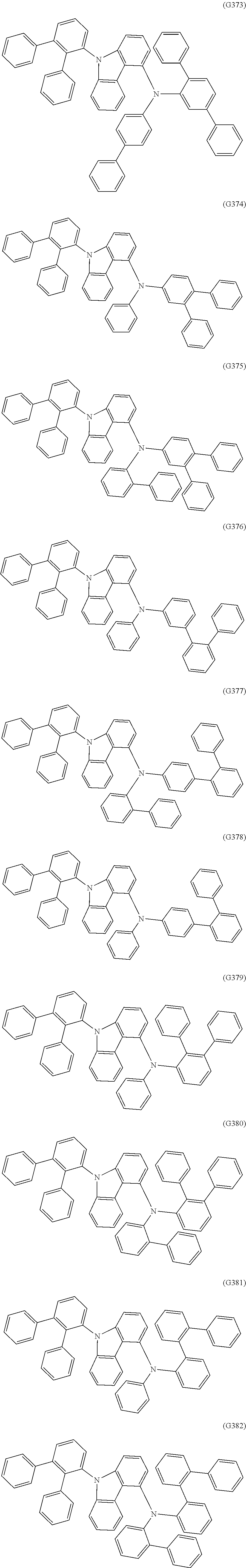

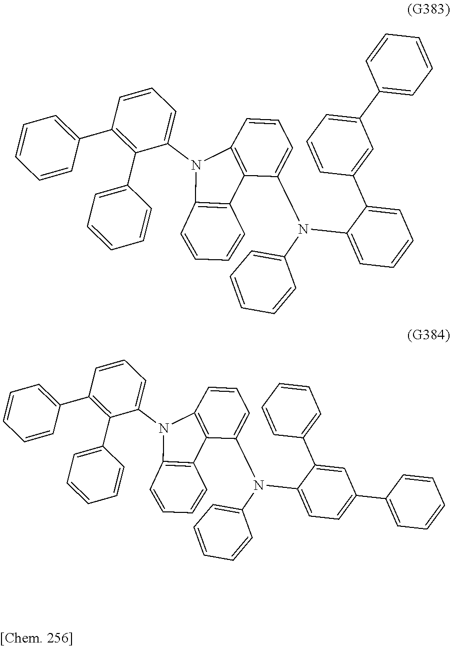 [SMILES: c1ccc(-c2cccc(-c3ccccc3N(c3ccccc3)c3cccc4c3c3ccccc3n4-c3cccc(-c4ccccc4)c3-c3ccccc3)c2)cc1 ; c1ccc(-c2ccc(N(c3ccccc3)c3cccc4c3c3ccccc3n4-c3cccc(-c4ccccc4)c3-c3ccccc3)c(-c3ccccc3)c2)cc1]

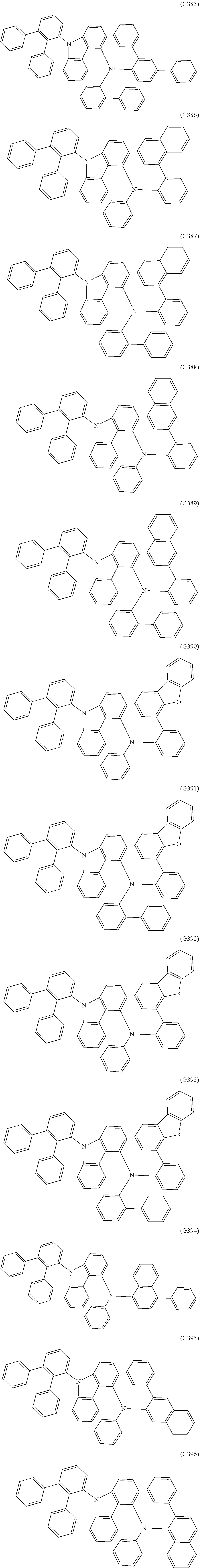

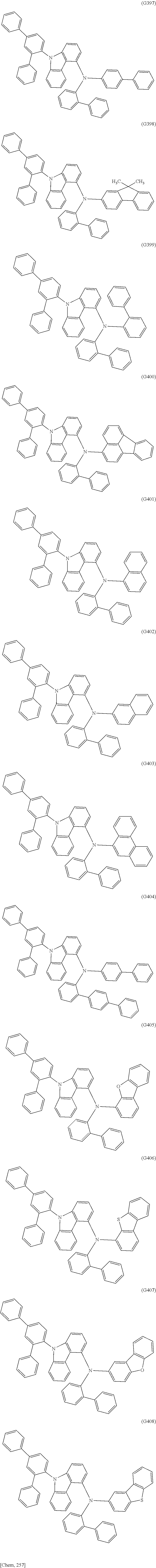

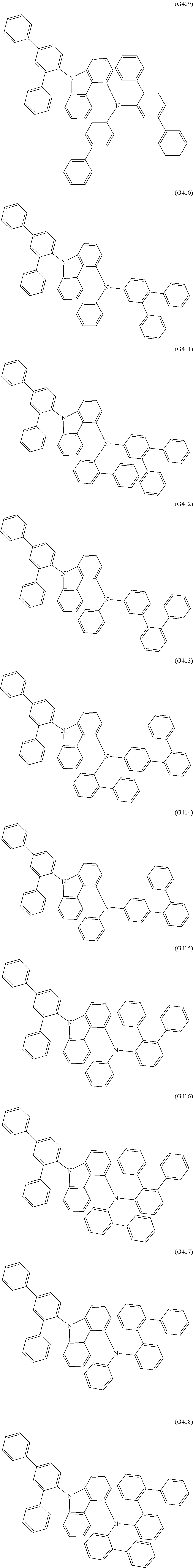

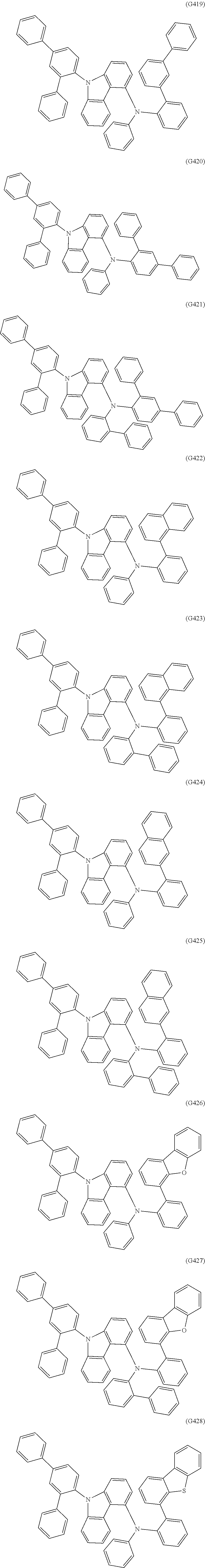

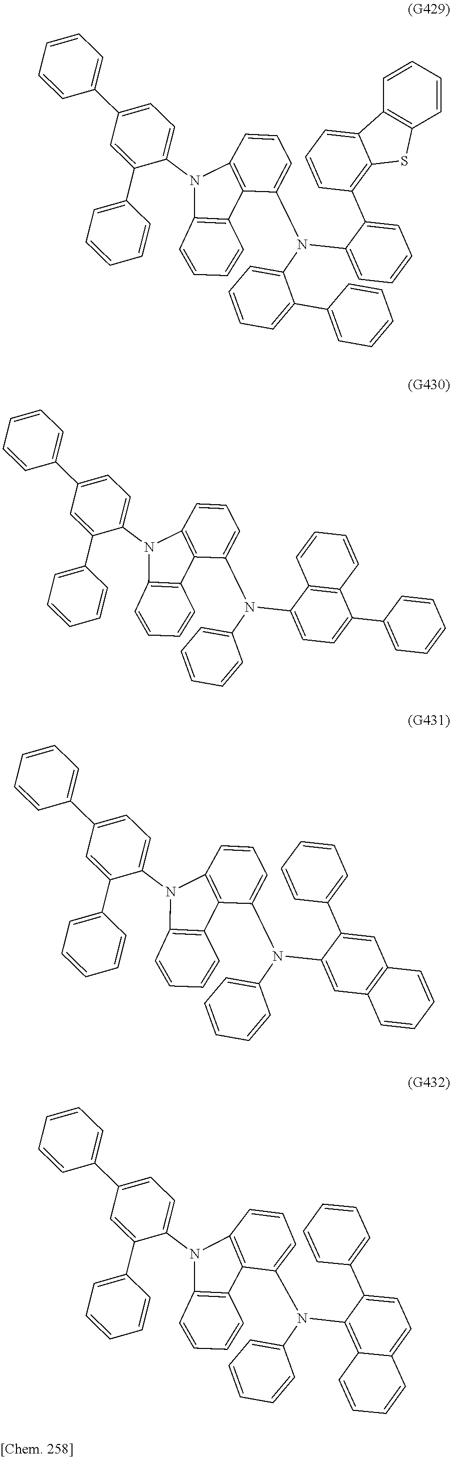 [SMILES: c1ccc(-c2ccc(-n3c4ccccc4c4c(N(c5ccccc5-c5ccccc5)c5ccccc5-c5cccc6c5sc5ccccc56)cccc43)c(-c3ccccc3)c2)cc1 ; c1ccc(-c2ccc(-n3c4ccccc4c4c(N(c5ccccc5)c5ccc(-c6ccccc6)c6ccccc56)cccc43)c(-c3ccccc3)c2)cc1 ; c1ccc(-c2ccc(-n3c4ccccc4c4c(N(c5ccccc5)c5cc6ccccc6cc5-c5ccccc5)cccc43)c(-c3ccccc3)c2)cc1 ; c1ccc(-c2ccc(-n3c4ccccc4c4c(N(c5ccccc5)c5c(-c6ccccc6)ccc6ccccc56)cccc43)c(-c3ccccc3)c2)cc1]

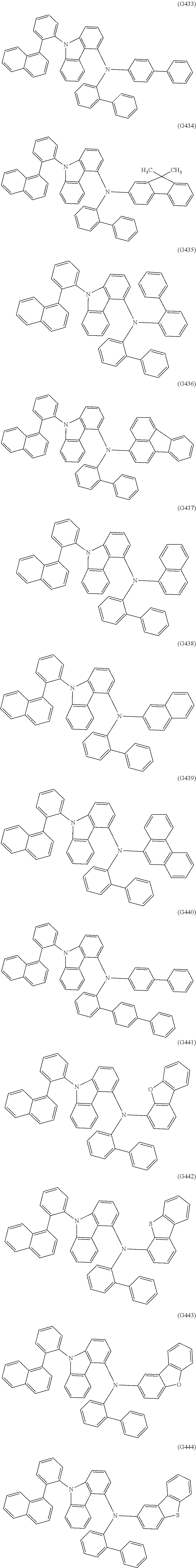

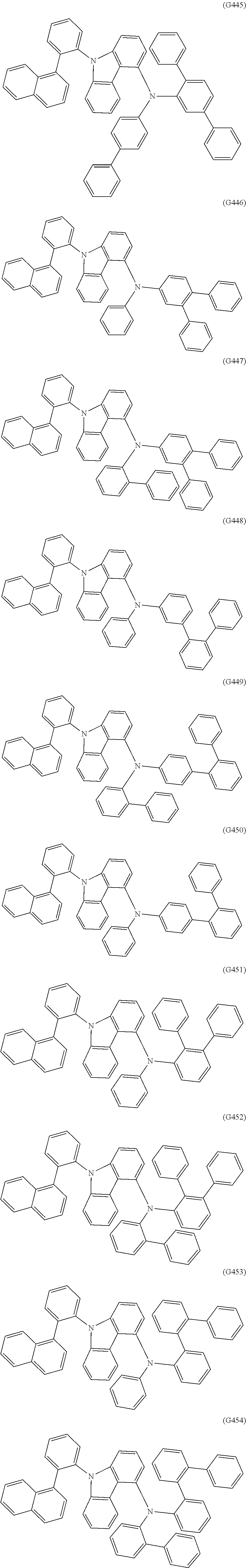

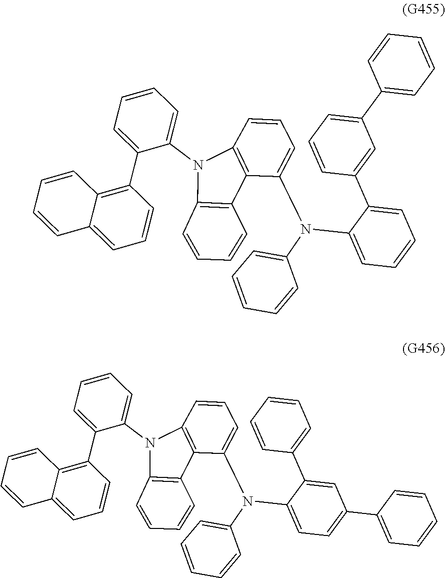 [SMILES: c1ccc(-c2cccc(-c3ccccc3N(c3ccccc3)c3cccc4c3c3ccccc3n4-c3ccccc3-c3cccc4ccccc34)c2)cc1 ; c1ccc(-c2ccc(N(c3ccccc3)c3cccc4c3c3ccccc3n4-c3ccccc3-c3cccc4ccccc34)c(-c3ccccc3)c2)cc1]

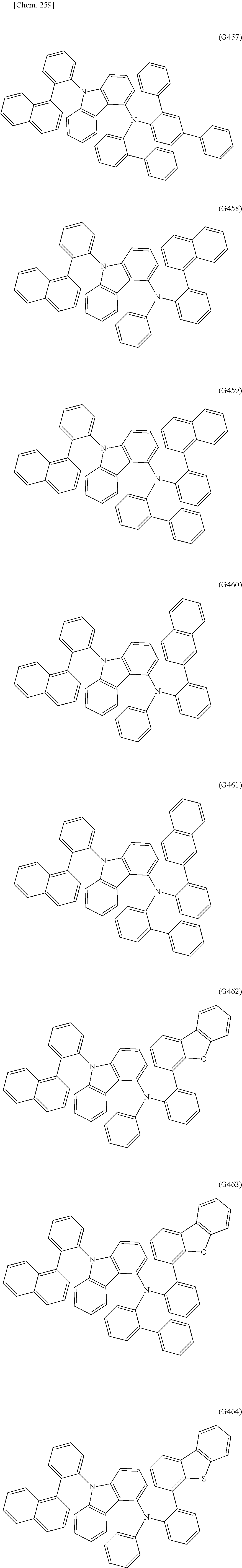

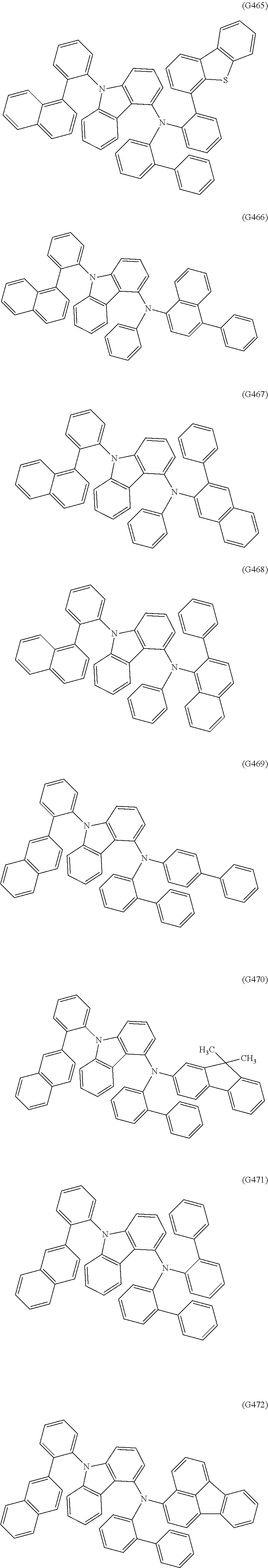

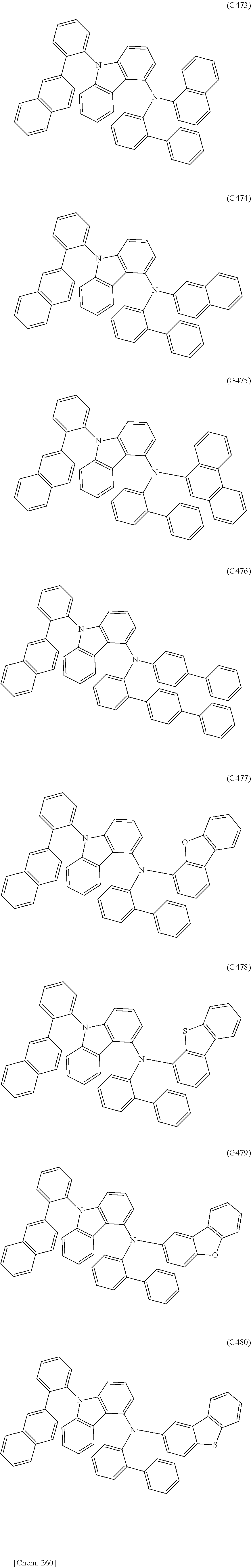

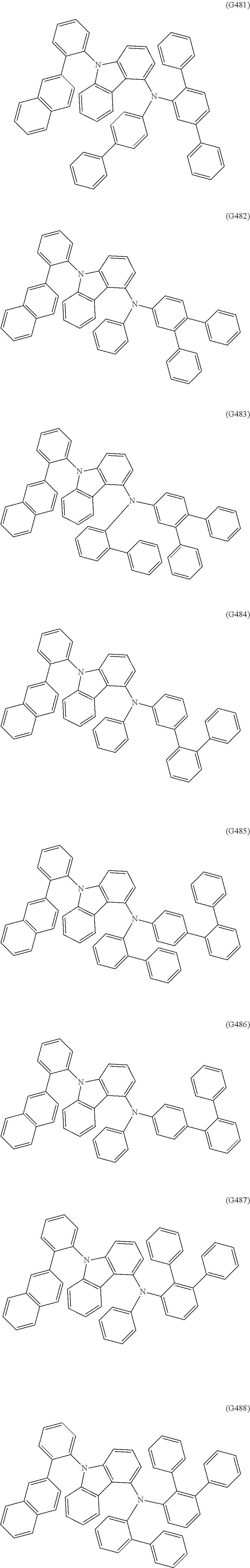

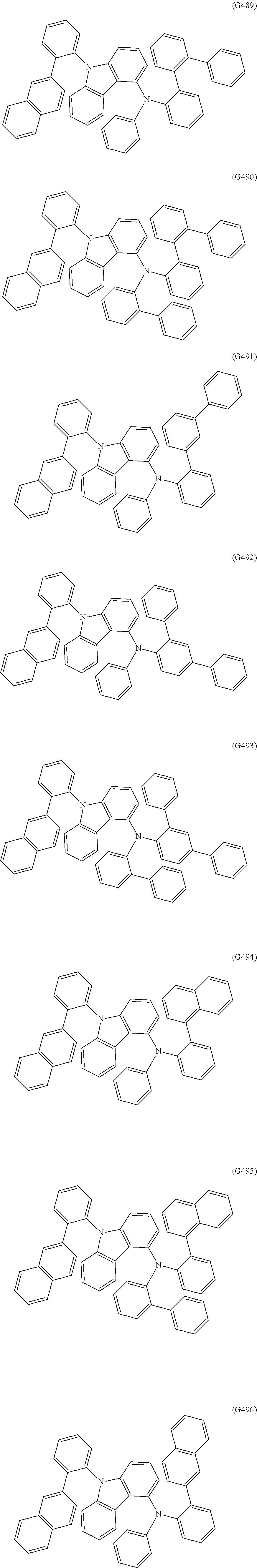

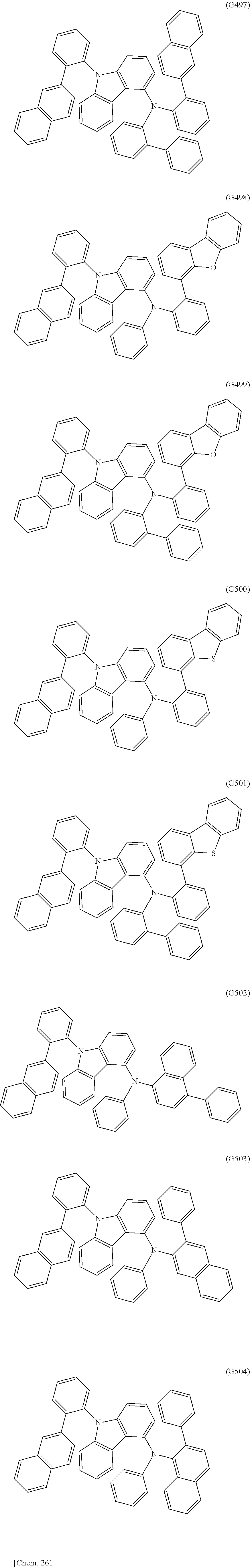

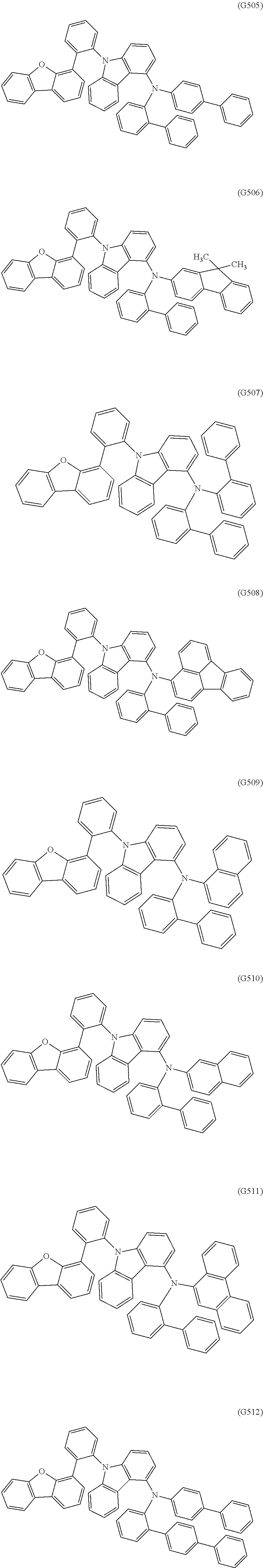

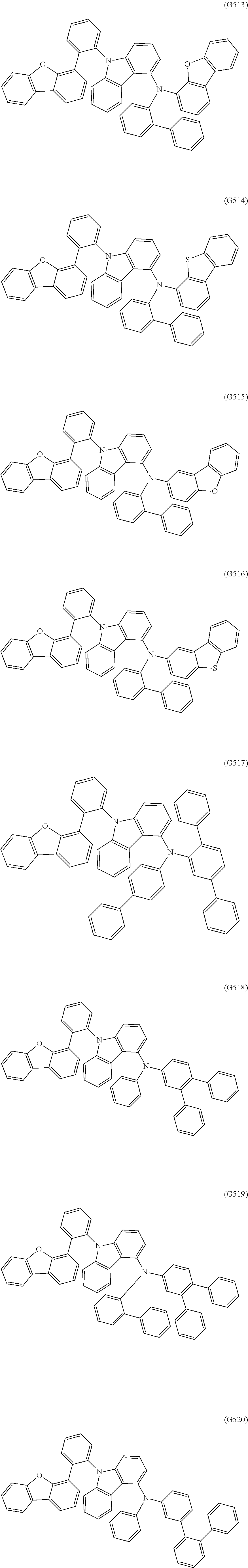

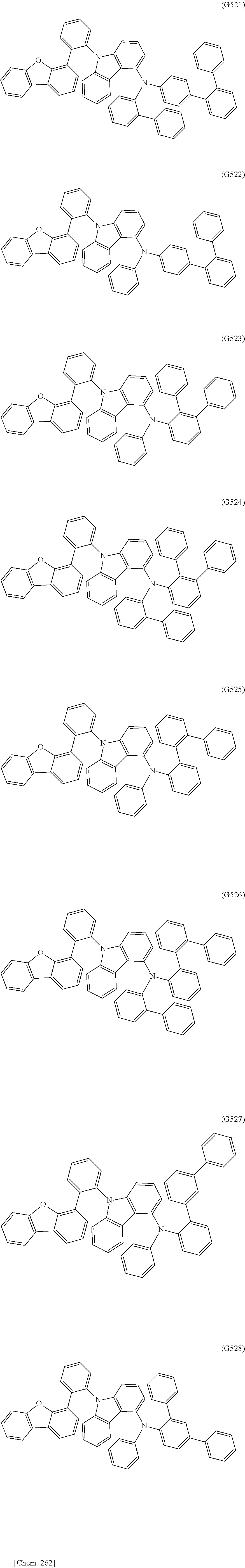

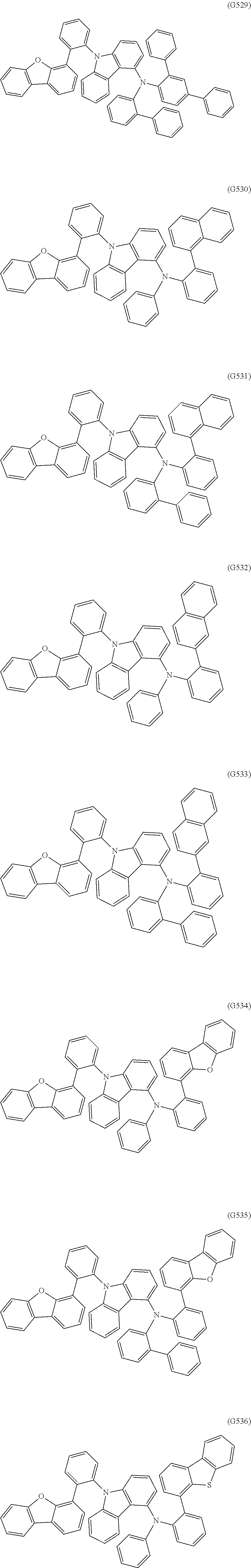

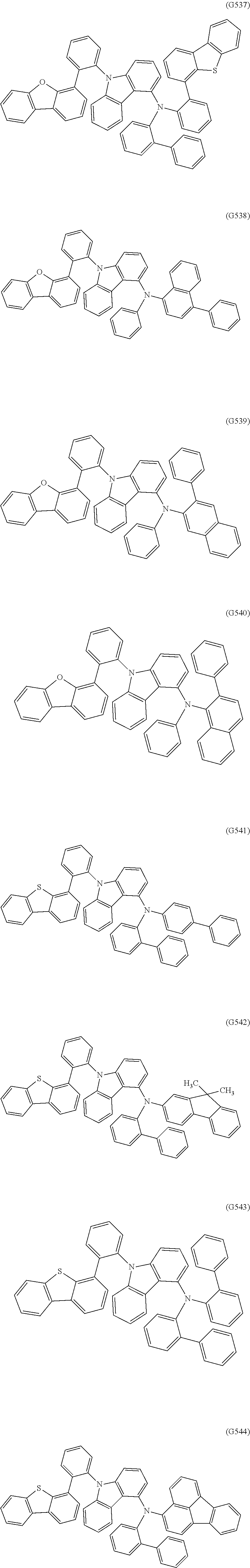

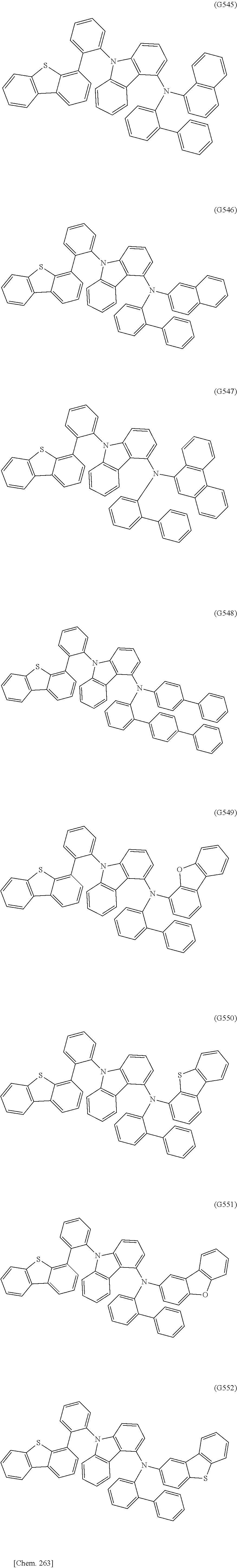

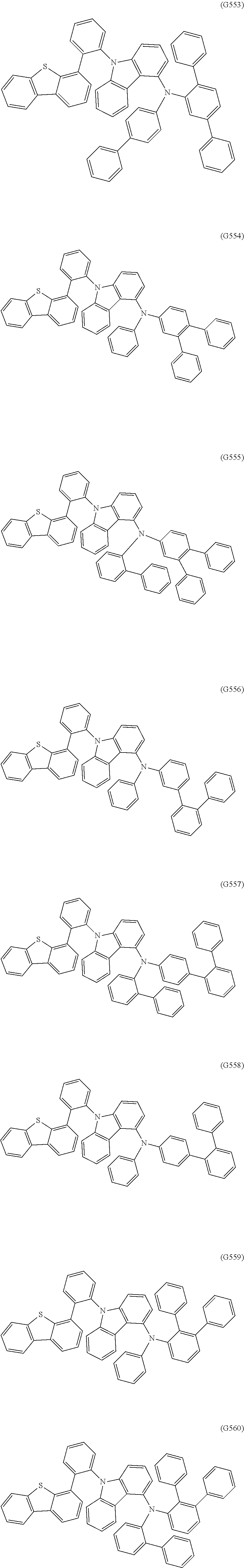

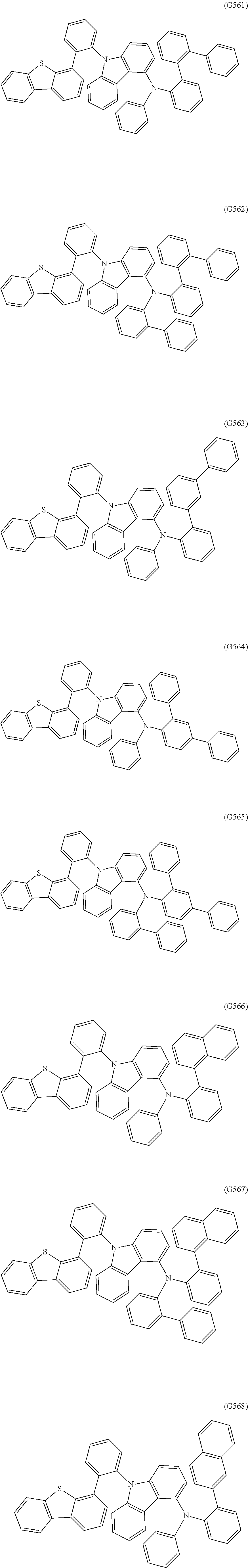

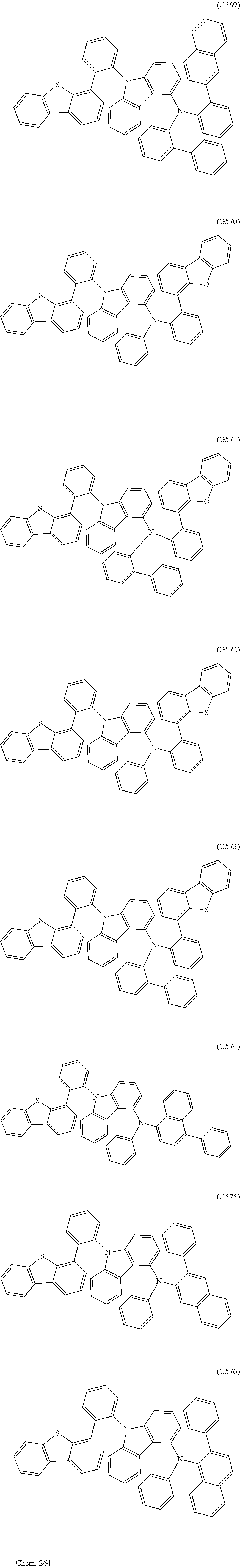

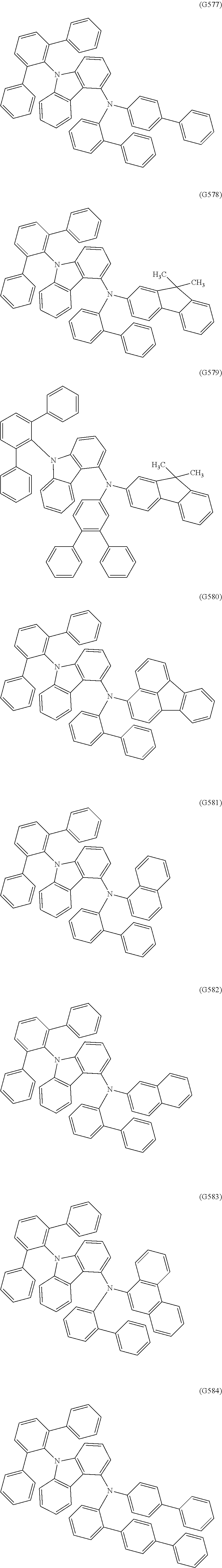

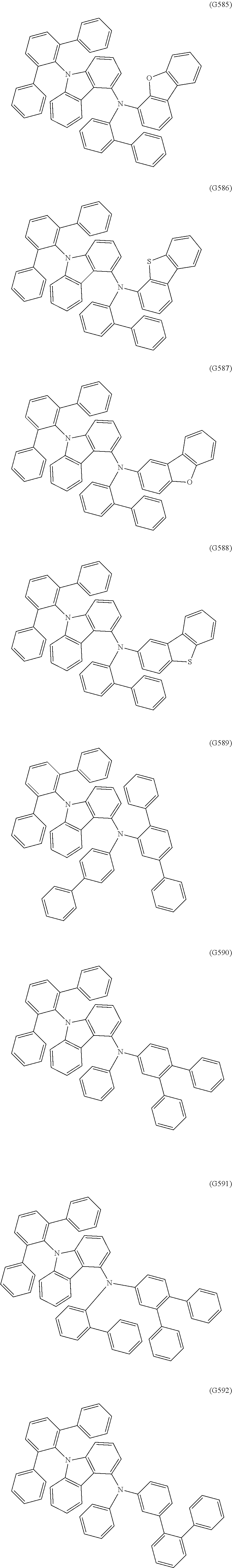

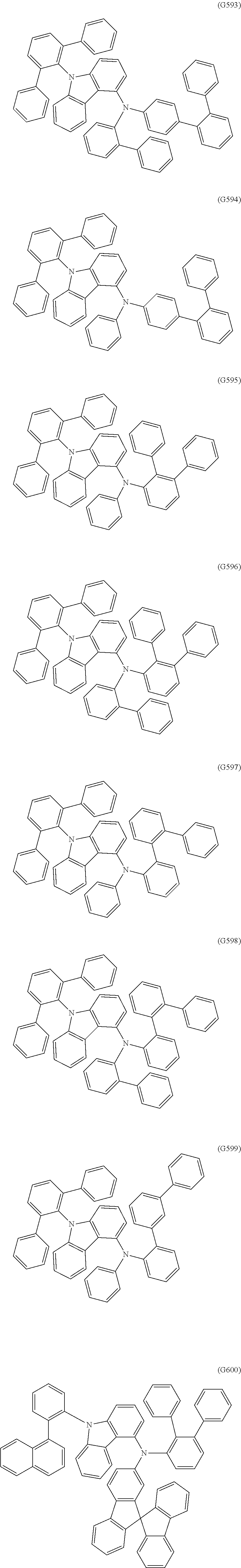

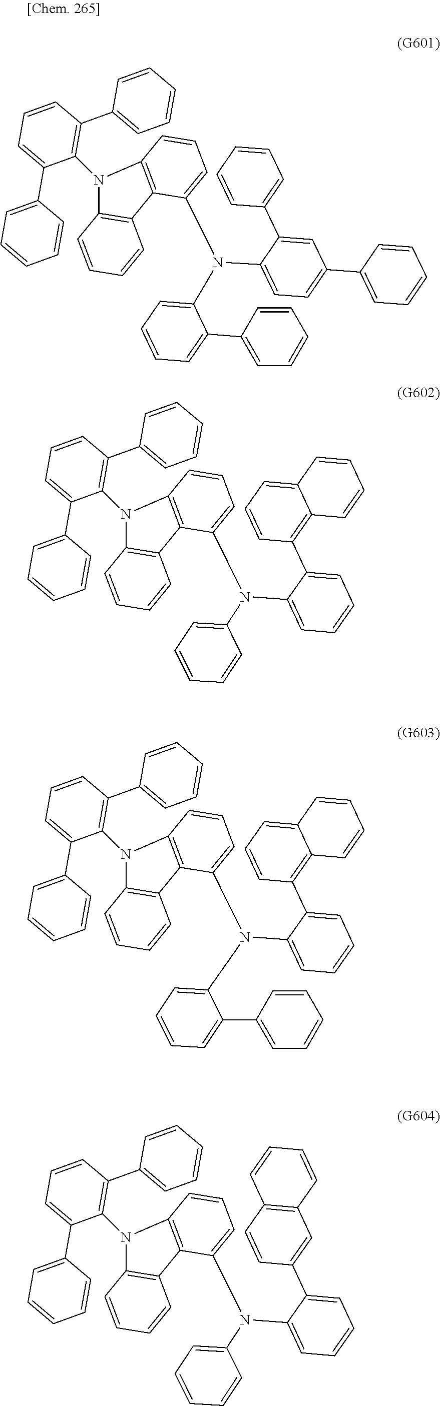

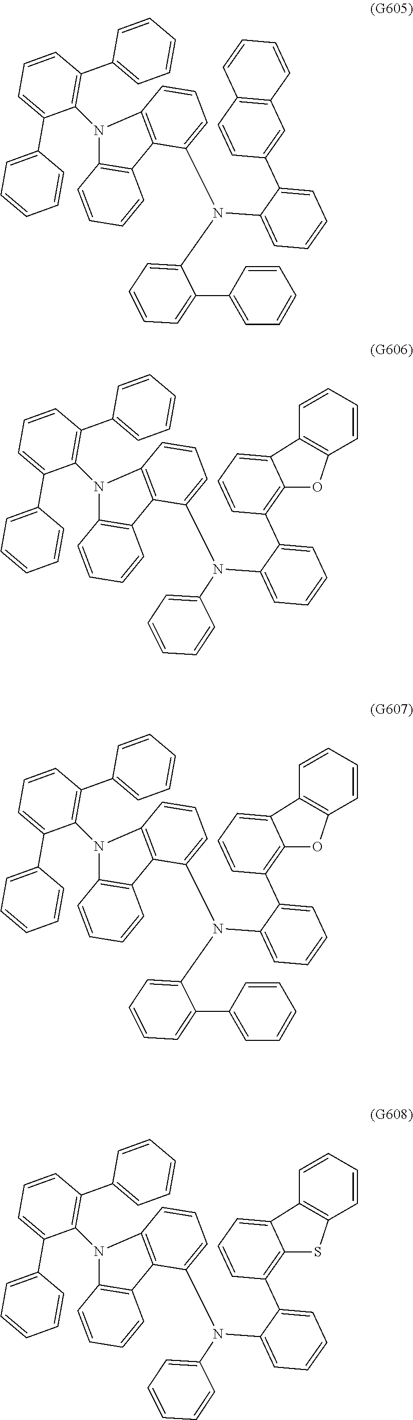 [SMILES: c1ccc(-c2ccccc2N(c2ccccc2-c2ccc3ccccc3c2)c2cccc3c2c2ccccc2n3-c2c(-c3ccccc3)cccc2-c2ccccc2)cc1 ; c1ccc(-c2cccc(-c3ccccc3)c2-n2c3ccccc3c3c(N(c4ccccc4)c4ccccc4-c4cccc5c4oc4ccccc45)cccc32)cc1 ; c1ccc(-c2ccccc2N(c2ccccc2-c2cccc3c2oc2ccccc23)c2cccc3c2c2ccccc2n3-c2c(-c3ccccc3)cccc2-c2ccccc2)cc1 ; c1ccc(-c2cccc(-c3ccccc3)c2-n2c3ccccc3c3c(N(c4ccccc4)c4ccccc4-c4cccc5c4sc4ccccc45)cccc32)cc1]

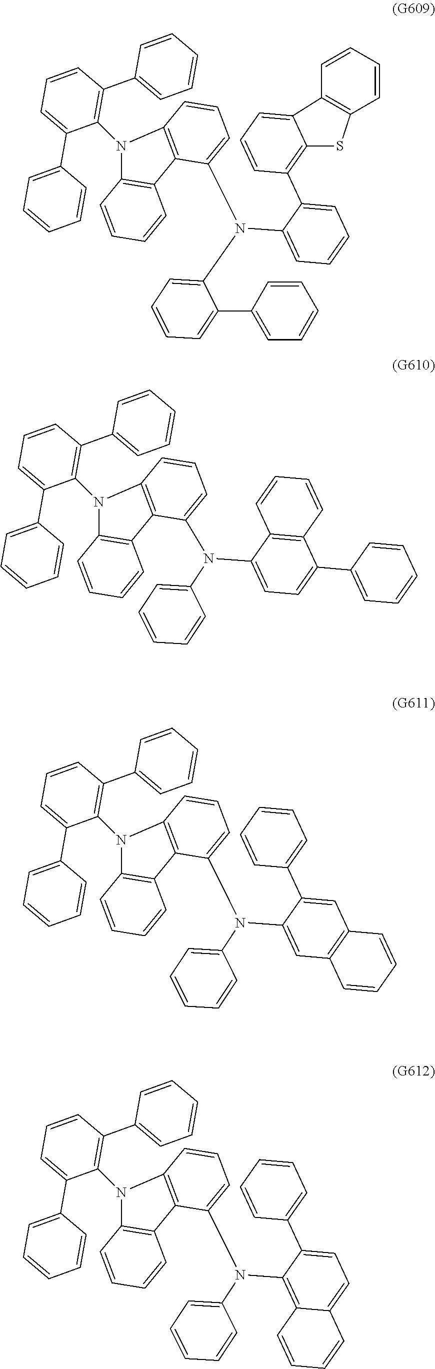 [SMILES: c1ccc(-c2ccccc2N(c2ccccc2-c2cccc3c2sc2ccccc23)c2cccc3c2c2ccccc2n3-c2c(-c3ccccc3)cccc2-c2ccccc2)cc1 ; c1ccc(-c2cccc(-c3ccccc3)c2-n2c3ccccc3c3c(N(c4ccccc4)c4ccc(-c5ccccc5)c5ccccc45)cccc32)cc1 ; c1ccc(-c2cc3ccccc3cc2N(c2ccccc2)c2cccc3c2c2ccccc2n3-c2c(-c3ccccc3)cccc2-c2ccccc2)cc1 ; c1ccc(-c2cccc(-c3ccccc3)c2-n2c3ccccc3c3c(N(c4ccccc4)c4c(-c5ccccc5)ccc5ccccc45)cccc32)cc1]

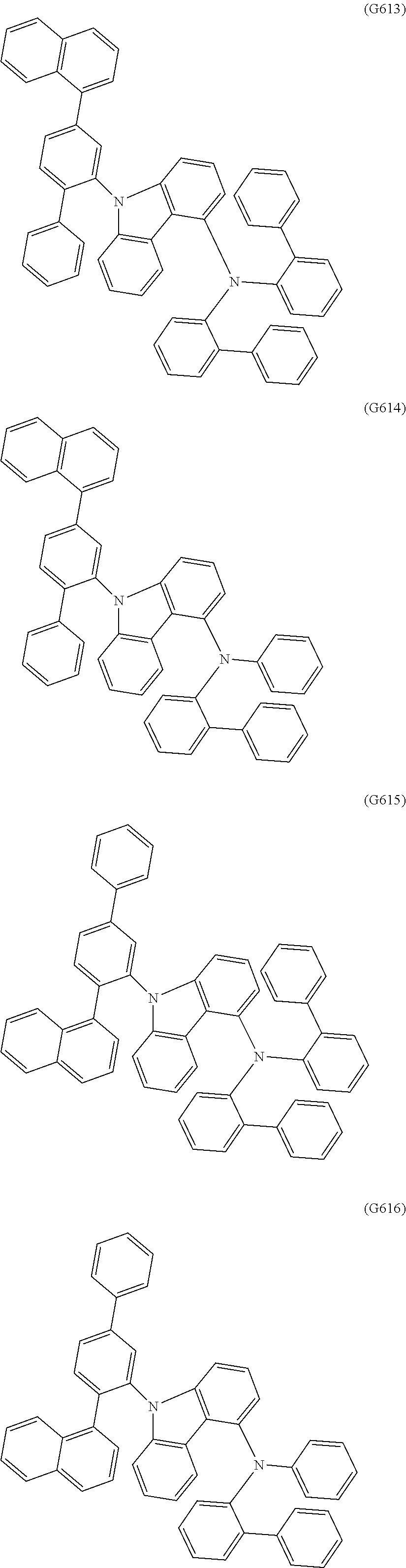 [SMILES: c1ccc(-c2ccccc2N(c2ccccc2-c2ccccc2)c2cccc3c2c2ccccc2n3-c2cc(-c3cccc4ccccc34)ccc2-c2ccccc2)cc1 ; c1ccc(-c2ccccc2N(c2ccccc2)c2cccc3c2c2ccccc2n3-c2cc(-c3cccc4ccccc34)ccc2-c2ccccc2)cc1 ; c1ccc(-c2ccc(-c3cccc4ccccc34)c(-n3c4ccccc4c4c(N(c5ccccc5-c5ccccc5)c5ccccc5-c5ccccc5)cccc43)c2)cc1 ; c1ccc(-c2ccc(-c3cccc4ccccc34)c(-n3c4ccccc4c4c(N(c5ccccc5)c5ccccc5-c5ccccc5)cccc43)c2)cc1]

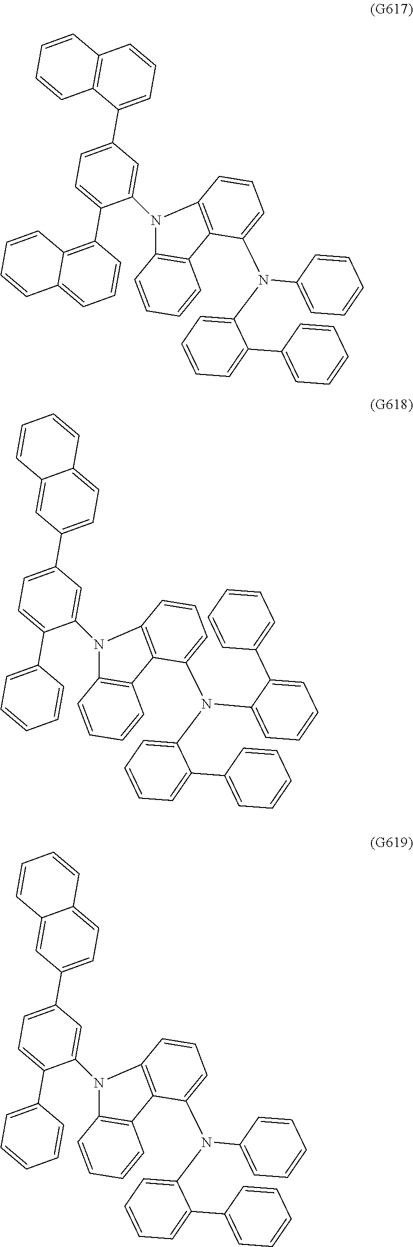 [SMILES: c1ccc(-c2ccccc2N(c2ccccc2)c2cccc3c2c2ccccc2n3-c2cc(-c3cccc4ccccc34)ccc2-c2cccc3ccccc23)cc1 ; c1ccc(-c2ccccc2N(c2ccccc2-c2ccccc2)c2cccc3c2c2ccccc2n3-c2cc(-c3ccc4ccccc4c3)ccc2-c2ccccc2)cc1 ; c1ccc(-c2ccccc2N(c2ccccc2)c2cccc3c2c2ccccc2n3-c2cc(-c3ccc4ccccc4c3)ccc2-c2ccccc2)cc1]

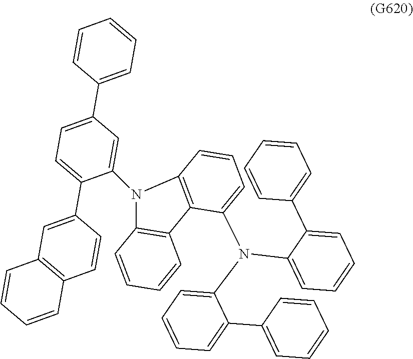 [SMILES: c1ccc(-c2ccc(-c3ccc4ccccc4c3)c(-n3c4ccccc4c4c(N(c5ccccc5-c5ccccc5)c5ccccc5-c5ccccc5)cccc43)c2)cc1]

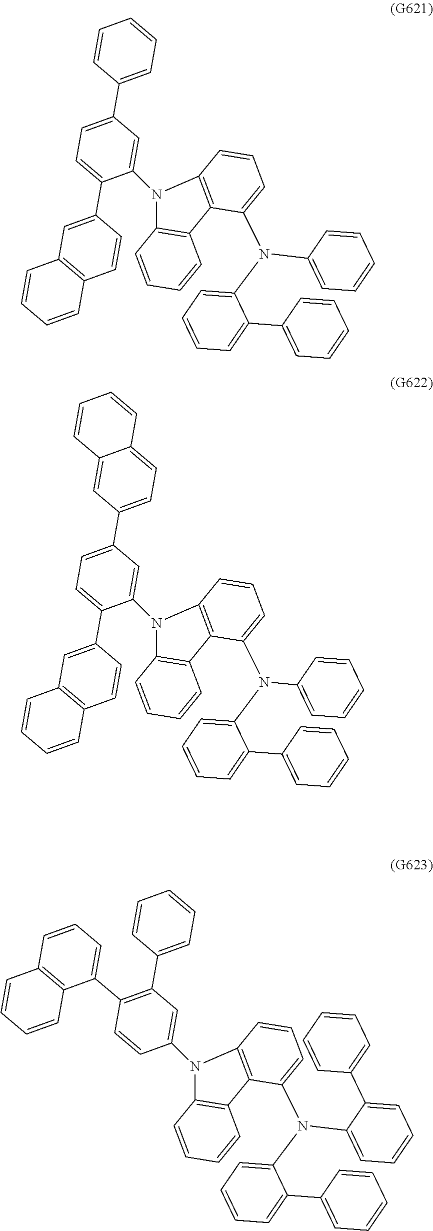 [SMILES: c1ccc(-c2ccc(-c3ccc4ccccc4c3)c(-n3c4ccccc4c4c(N(c5ccccc5)c5ccccc5-c5ccccc5)cccc43)c2)cc1 ; c1ccc(-c2ccccc2N(c2ccccc2)c2cccc3c2c2ccccc2n3-c2cc(-c3ccc4ccccc4c3)ccc2-c2ccc3ccccc3c2)cc1 ; c1ccc(-c2cc(-n3c4ccccc4c4c(N(c5ccccc5-c5ccccc5)c5ccccc5-c5ccccc5)cccc43)ccc2-c2cccc3ccccc23)cc1]

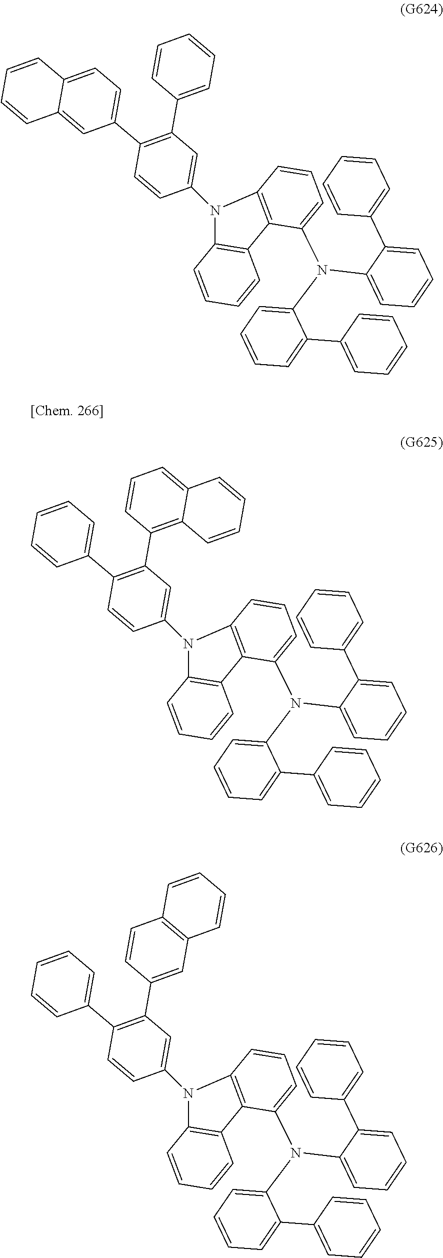 [SMILES: c1ccc(-c2cc(-n3c4ccccc4c4c(N(c5ccccc5-c5ccccc5)c5ccccc5-c5ccccc5)cccc43)ccc2-c2ccc3ccccc3c2)cc1 ; c1ccc(-c2ccc(-n3c4ccccc4c4c(N(c5ccccc5-c5ccccc5)c5ccccc5-c5ccccc5)cccc43)cc2-c2cccc3ccccc23)cc1 ; c1ccc(-c2ccc(-n3c4ccccc4c4c(N(c5ccccc5-c5ccccc5)c5ccccc5-c5ccccc5)cccc43)cc2-c2ccc3ccccc3c2)cc1]

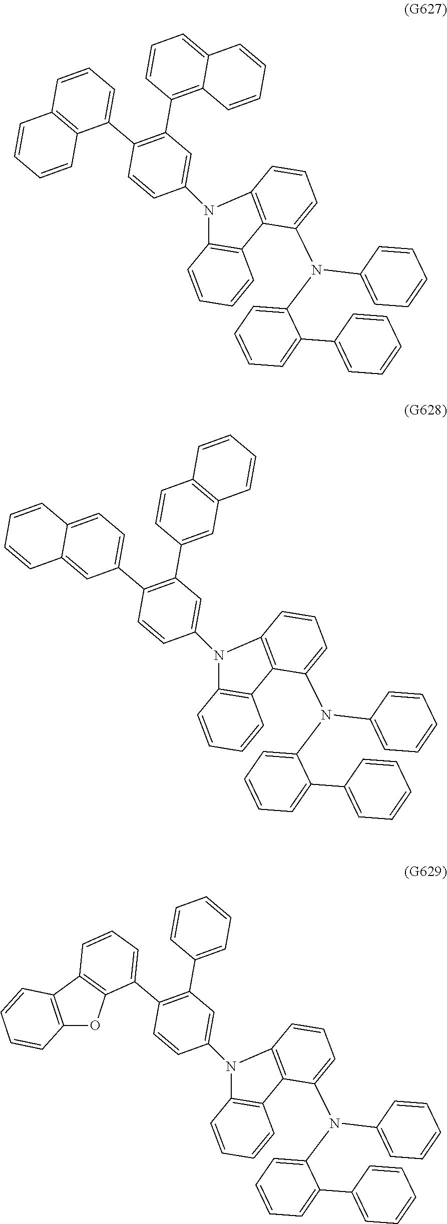 [SMILES: c1ccc(-c2ccccc2N(c2ccccc2)c2cccc3c2c2ccccc2n3-c2ccc(-c3cccc4ccccc34)c(-c3cccc4ccccc34)c2)cc1 ; c1ccc(-c2ccccc2N(c2ccccc2)c2cccc3c2c2ccccc2n3-c2ccc(-c3ccc4ccccc4c3)c(-c3ccc4ccccc4c3)c2)cc1 ; c1ccc(-c2cc(-n3c4ccccc4c4c(N(c5ccccc5)c5ccccc5-c5ccccc5)cccc43)ccc2-c2cccc3c2oc2ccccc23)cc1]

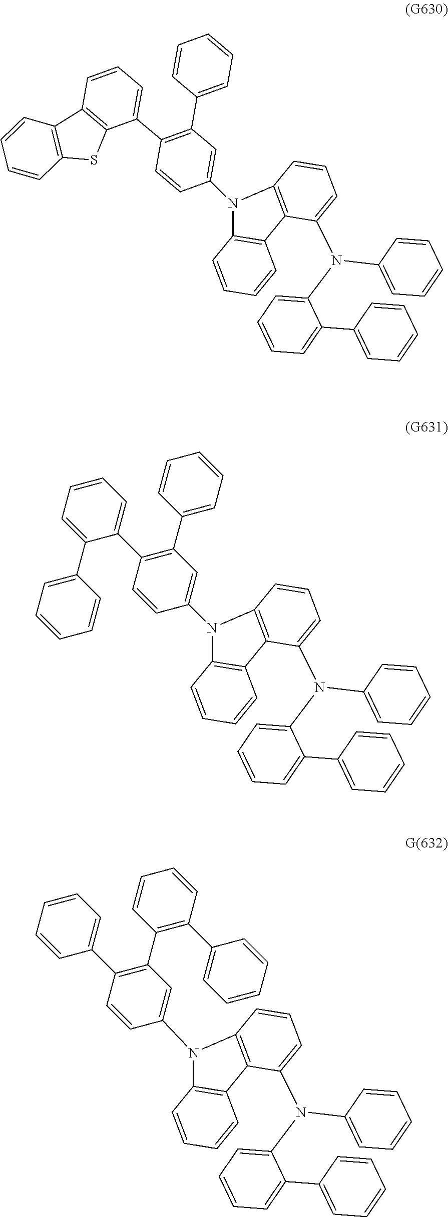 [SMILES: c1ccc(-c2cc(-n3c4ccccc4c4c(N(c5ccccc5)c5ccccc5-c5ccccc5)cccc43)ccc2-c2cccc3c2sc2ccccc23)cc1 ; c1ccc(-c2ccccc2-c2ccc(-n3c4ccccc4c4c(N(c5ccccc5)c5ccccc5-c5ccccc5)cccc43)cc2-c2ccccc2)cc1 ; c1ccc(-c2ccccc2-c2cc(-n3c4ccccc4c4c(N(c5ccccc5)c5ccccc5-c5ccccc5)cccc43)ccc2-c2ccccc2)cc1]

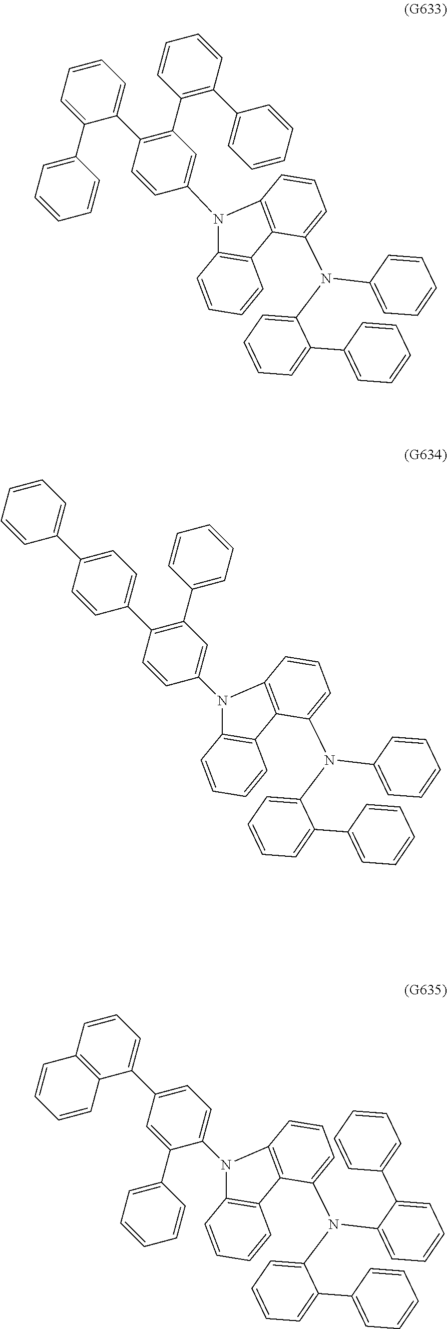 [SMILES: c1ccc(-c2ccccc2-c2ccc(-n3c4ccccc4c4c(N(c5ccccc5)c5ccccc5-c5ccccc5)cccc43)cc2-c2ccccc2-c2ccccc2)cc1 ; c1ccc(-c2ccc(-c3ccc(-n4c5ccccc5c5c(N(c6ccccc6)c6ccccc6-c6ccccc6)cccc54)cc3-c3ccccc3)cc2)cc1 ; c1ccc(-c2ccccc2N(c2ccccc2-c2ccccc2)c2cccc3c2c2ccccc2n3-c2ccc(-c3cccc4ccccc34)cc2-c2ccccc2)cc1]

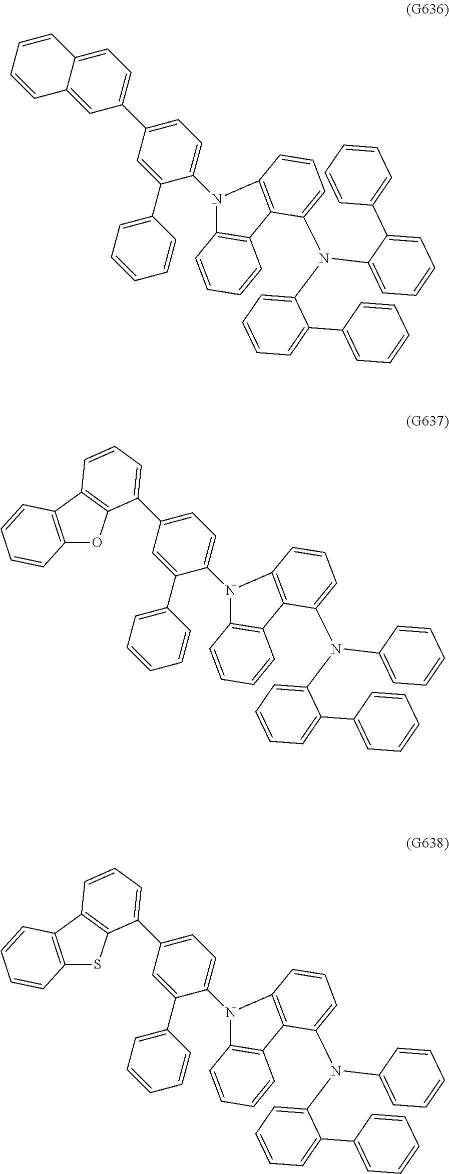 [SMILES: c1ccc(-c2ccccc2N(c2ccccc2-c2ccccc2)c2cccc3c2c2ccccc2n3-c2ccc(-c3ccc4ccccc4c3)cc2-c2ccccc2)cc1 ; c1ccc(-c2ccccc2N(c2ccccc2)c2cccc3c2c2ccccc2n3-c2ccc(-c3cccc4c3oc3ccccc34)cc2-c2ccccc2)cc1 ; c1ccc(-c2ccccc2N(c2ccccc2)c2cccc3c2c2ccccc2n3-c2ccc(-c3cccc4c3sc3ccccc34)cc2-c2ccccc2)cc1]

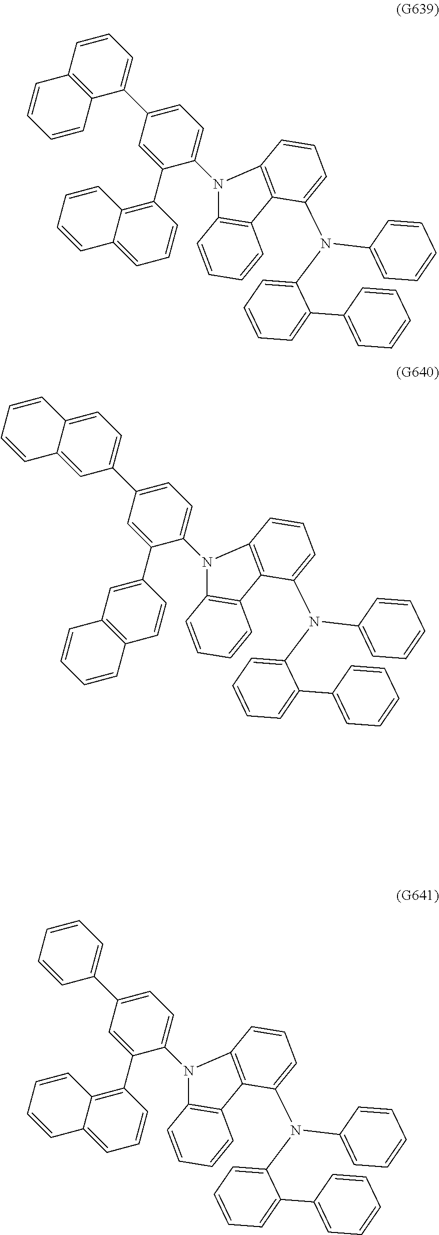 [SMILES: c1ccc(-c2ccccc2N(c2ccccc2)c2cccc3c2c2ccccc2n3-c2ccc(-c3cccc4ccccc34)cc2-c2cccc3ccccc23)cc1 ; c1ccc(-c2ccccc2N(c2ccccc2)c2cccc3c2c2ccccc2n3-c2ccc(-c3ccc4ccccc4c3)cc2-c2ccc3ccccc3c2)cc1 ; c1ccc(-c2ccc(-n3c4ccccc4c4c(N(c5ccccc5)c5ccccc5-c5ccccc5)cccc43)c(-c3cccc4ccccc34)c2)cc1]

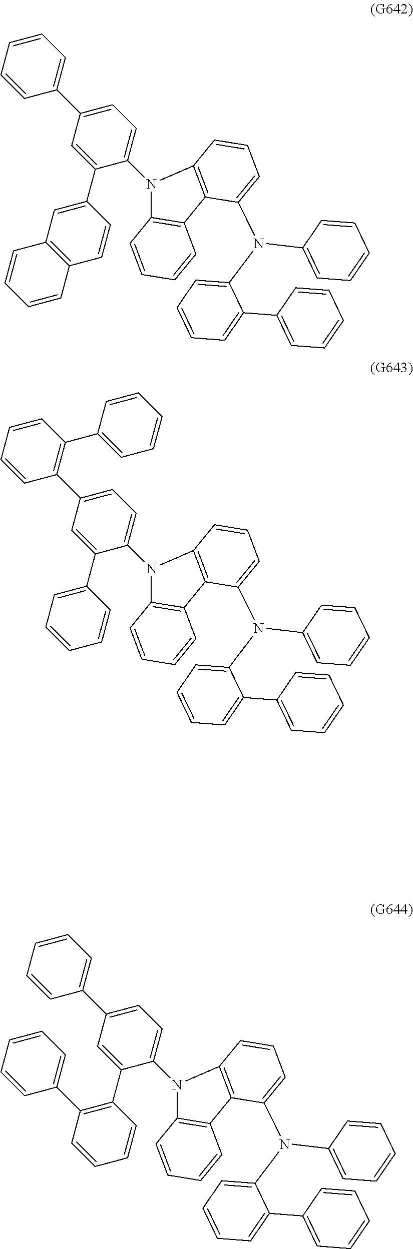 [SMILES: c1ccc(-c2ccc(-n3c4ccccc4c4c(N(c5ccccc5)c5ccccc5-c5ccccc5)cccc43)c(-c3ccc4ccccc4c3)c2)cc1 ; c1ccc(-c2ccccc2-c2ccc(-n3c4ccccc4c4c(N(c5ccccc5)c5ccccc5-c5ccccc5)cccc43)c(-c3ccccc3)c2)cc1 ; c1ccc(-c2ccc(-n3c4ccccc4c4c(N(c5ccccc5)c5ccccc5-c5ccccc5)cccc43)c(-c3ccccc3-c3ccccc3)c2)cc1]

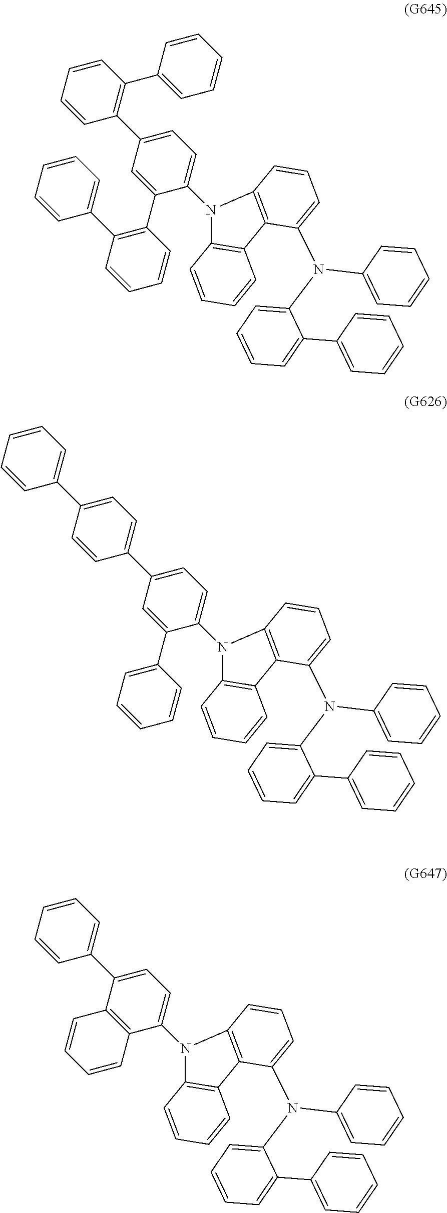 [SMILES: c1ccc(-c2ccccc2-c2ccc(-n3c4ccccc4c4c(N(c5ccccc5)c5ccccc5-c5ccccc5)cccc43)c(-c3ccccc3-c3ccccc3)c2)cc1 ; c1ccc(-c2ccc(-c3ccc(-n4c5ccccc5c5c(N(c6ccccc6)c6ccccc6-c6ccccc6)cccc54)c(-c4ccccc4)c3)cc2)cc1 ; c1ccc(-c2ccccc2N(c2ccccc2)c2cccc3c2c2ccccc2n3-c2ccc(-c3ccccc3)c3ccccc23)cc1]

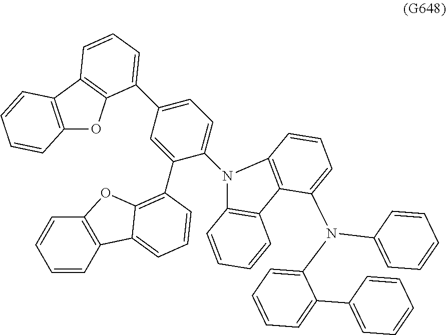 [SMILES: c1ccc(-c2ccccc2N(c2ccccc2)c2cccc3c2c2ccccc2n3-c2ccc(-c3cccc4c3oc3ccccc34)cc2-c2cccc3c2oc2ccccc23)cc1]

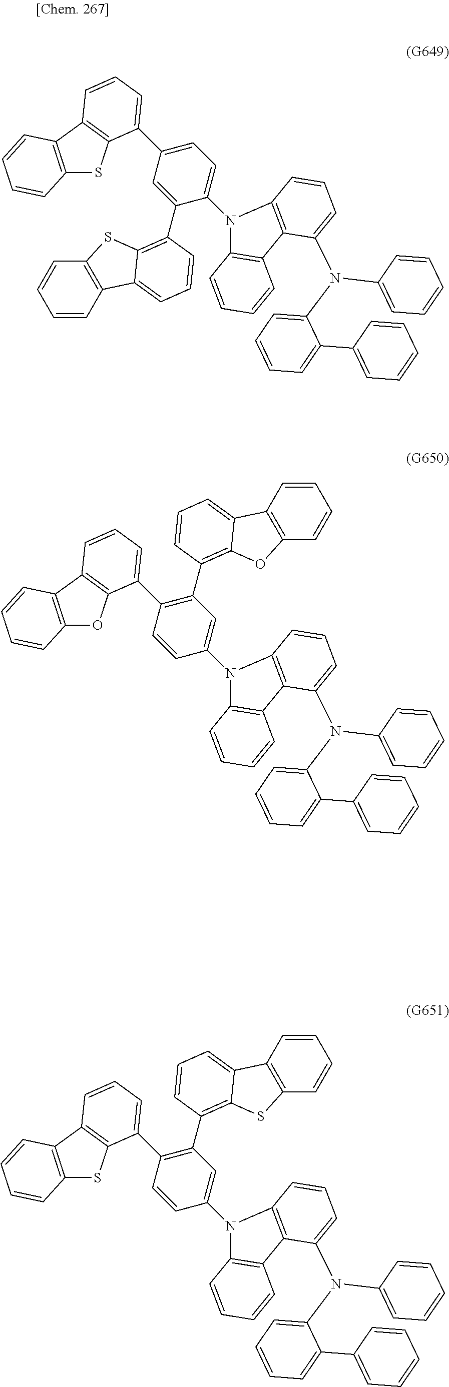 [SMILES: c1ccc(-c2ccccc2N(c2ccccc2)c2cccc3c2c2ccccc2n3-c2ccc(-c3cccc4c3sc3ccccc34)cc2-c2cccc3c2sc2ccccc23)cc1 ; c1ccc(-c2ccccc2N(c2ccccc2)c2cccc3c2c2ccccc2n3-c2ccc(-c3cccc4c3oc3ccccc34)c(-c3cccc4c3oc3ccccc34)c2)cc1 ; c1ccc(-c2ccccc2N(c2ccccc2)c2cccc3c2c2ccccc2n3-c2ccc(-c3cccc4c3sc3ccccc34)c(-c3cccc4c3sc3ccccc34)c2)cc1]

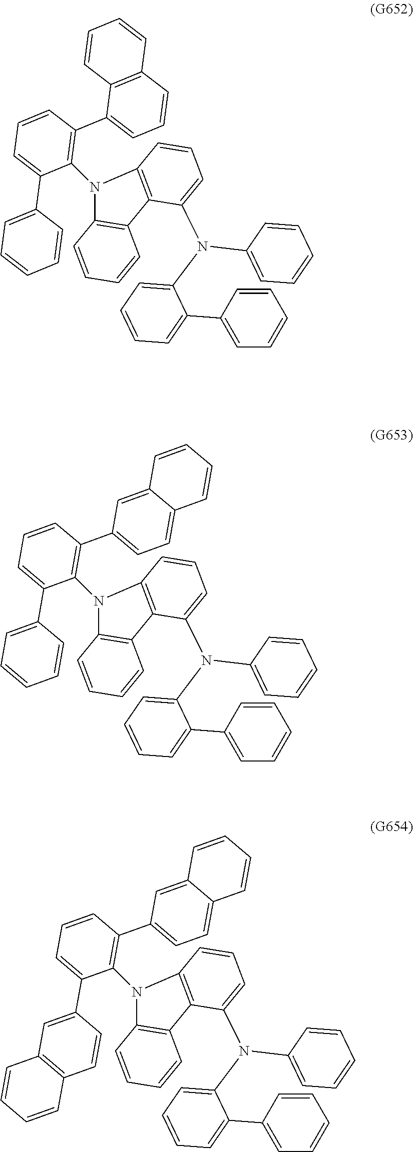 [SMILES: c1ccc(-c2ccccc2N(c2ccccc2)c2cccc3c2c2ccccc2n3-c2c(-c3ccccc3)cccc2-c2cccc3ccccc23)cc1 ; c1ccc(-c2ccccc2N(c2ccccc2)c2cccc3c2c2ccccc2n3-c2c(-c3ccccc3)cccc2-c2ccc3ccccc3c2)cc1 ; c1ccc(-c2ccccc2N(c2ccccc2)c2cccc3c2c2ccccc2n3-c2c(-c3ccc4ccccc4c3)cccc2-c2ccc3ccccc3c2)cc1]

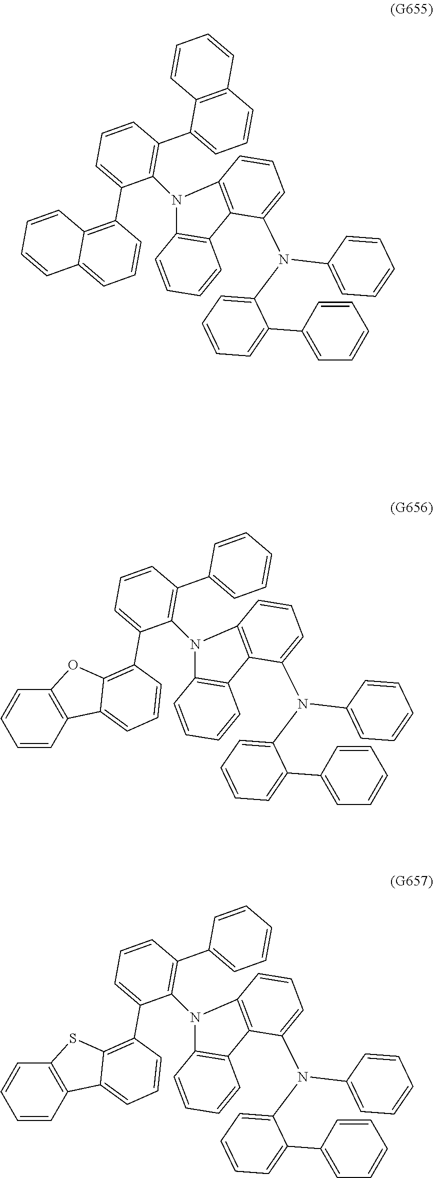 [SMILES: c1ccc(-c2ccccc2N(c2ccccc2)c2cccc3c2c2ccccc2n3-c2c(-c3cccc4ccccc34)cccc2-c2cccc3ccccc23)cc1 ; c1ccc(-c2ccccc2N(c2ccccc2)c2cccc3c2c2ccccc2n3-c2c(-c3ccccc3)cccc2-c2cccc3c2oc2ccccc23)cc1 ; c1ccc(-c2ccccc2N(c2ccccc2)c2cccc3c2c2ccccc2n3-c2c(-c3ccccc3)cccc2-c2cccc3c2sc2ccccc23)cc1]

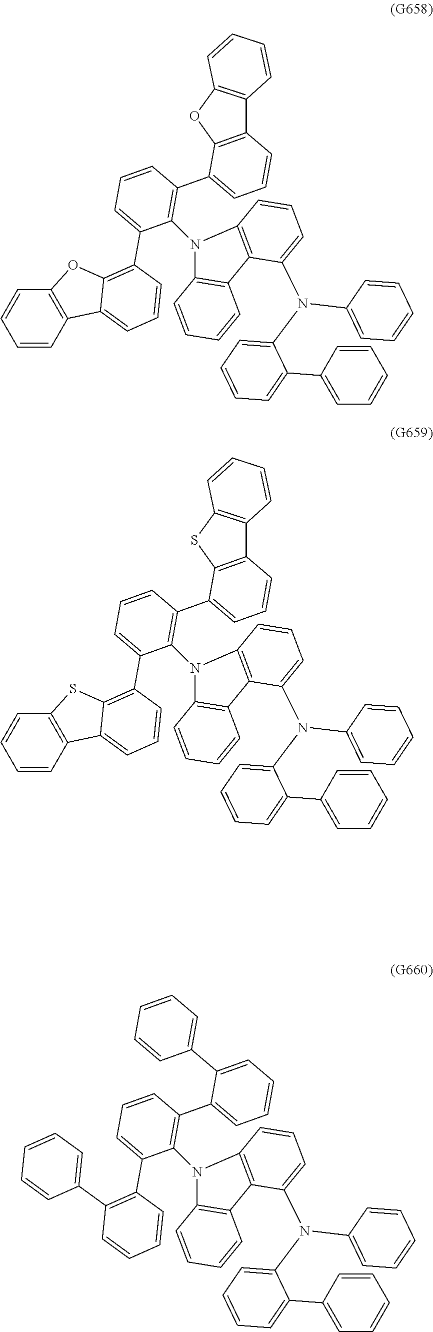 [SMILES: c1ccc(-c2ccccc2N(c2ccccc2)c2cccc3c2c2ccccc2n3-c2c(-c3cccc4c3oc3ccccc34)cccc2-c2cccc3c2oc2ccccc23)cc1 ; c1ccc(-c2ccccc2N(c2ccccc2)c2cccc3c2c2ccccc2n3-c2c(-c3cccc4c3sc3ccccc34)cccc2-c2cccc3c2sc2ccccc23)cc1 ; c1ccc(-c2ccccc2-c2cccc(-c3ccccc3-c3ccccc3)c2-n2c3ccccc3c3c(N(c4ccccc4)c4ccccc4-c4ccccc4)cccc32)cc1]

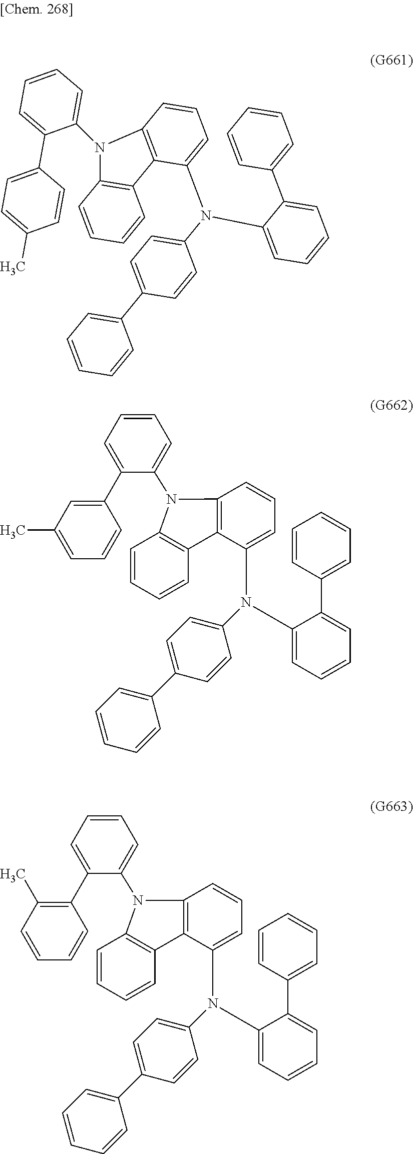

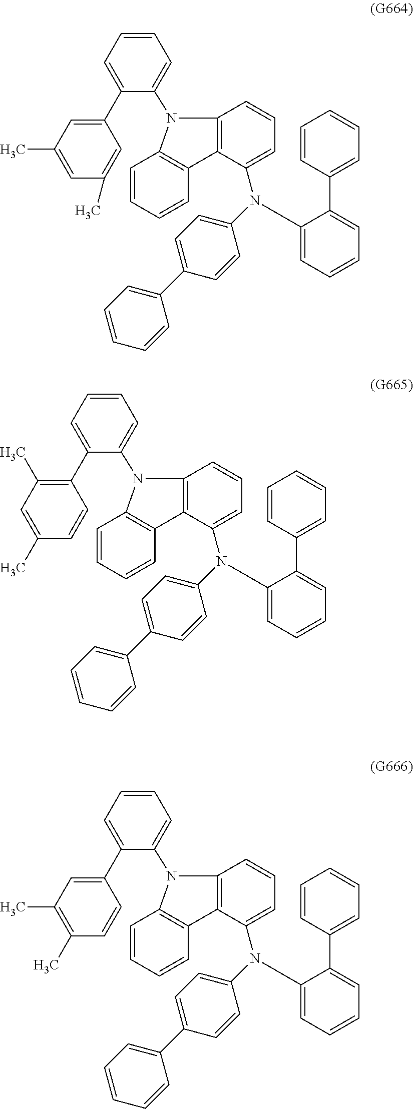 [SMILES: Cc1cc(C)cc(-c2ccccc2-n2c3ccccc3c3c(N(c4ccc(-c5ccccc5)cc4)c4ccccc4-c4ccccc4)cccc32)c1 ; Cc1ccc(-c2ccccc2-n2c3ccccc3c3c(N(c4ccc(-c5ccccc5)cc4)c4ccccc4-c4ccccc4)cccc32)c(C)c1 ; Cc1ccc(-c2ccccc2-n2c3ccccc3c3c(N(c4ccc(-c5ccccc5)cc4)c4ccccc4-c4ccccc4)cccc32)cc1C]

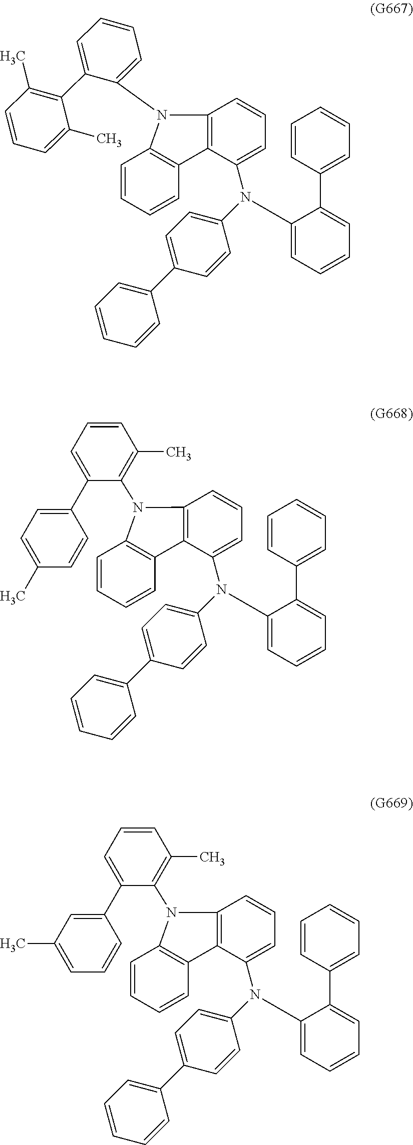 [SMILES: Cc1cccc(C)c1-c1ccccc1-n1c2ccccc2c2c(N(c3ccc(-c4ccccc4)cc3)c3ccccc3-c3ccccc3)cccc21 ; Cc1ccc(-c2cccc(C)c2-n2c3ccccc3c3c(N(c4ccc(-c5ccccc5)cc4)c4ccccc4-c4ccccc4)cccc32)cc1 ; Cc1cccc(-c2cccc(C)c2-n2c3ccccc3c3c(N(c4ccc(-c5ccccc5)cc4)c4ccccc4-c4ccccc4)cccc32)c1]

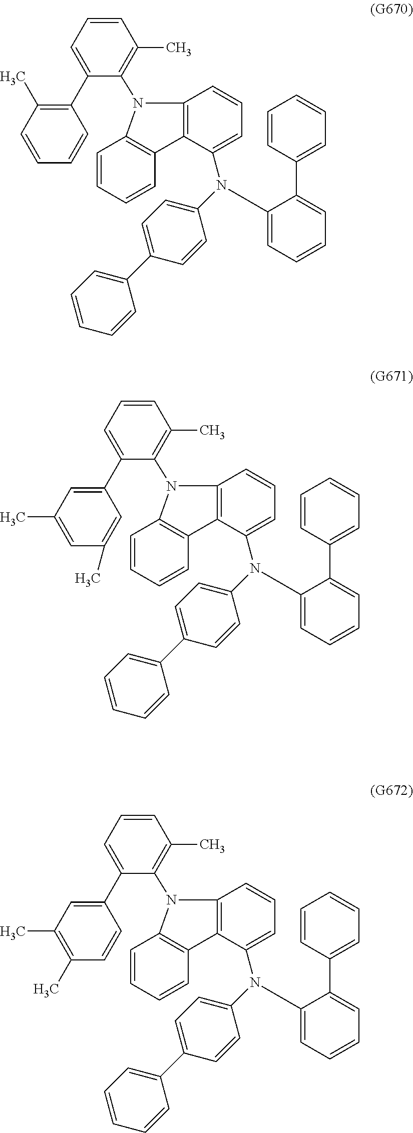 [SMILES: Cc1ccccc1-c1cccc(C)c1-n1c2ccccc2c2c(N(c3ccc(-c4ccccc4)cc3)c3ccccc3-c3ccccc3)cccc21 ; Cc1cc(C)cc(-c2cccc(C)c2-n2c3ccccc3c3c(N(c4ccc(-c5ccccc5)cc4)c4ccccc4-c4ccccc4)cccc32)c1 ; Cc1ccc(-c2cccc(C)c2-n2c3ccccc3c3c(N(c4ccc(-c5ccccc5)cc4)c4ccccc4-c4ccccc4)cccc32)cc1C]

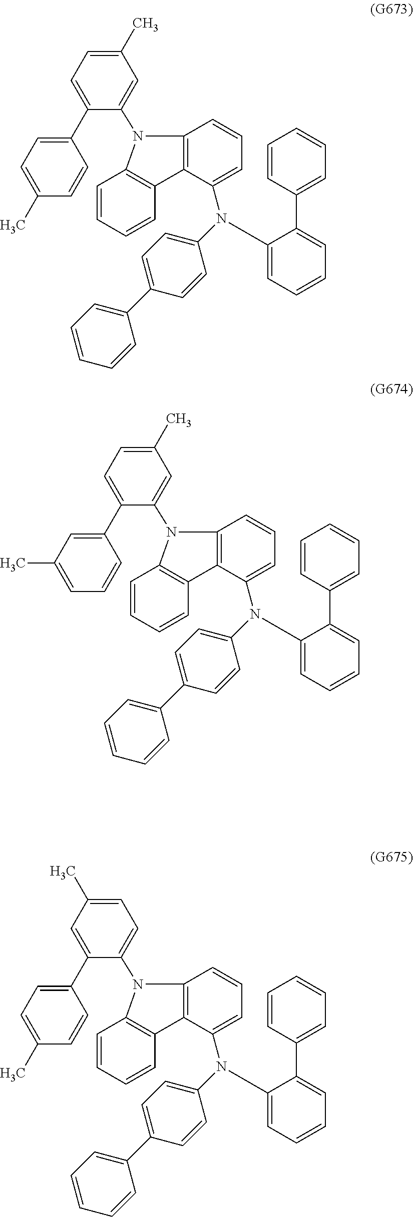 [SMILES: Cc1ccc(-c2ccc(C)cc2-n2c3ccccc3c3c(N(c4ccc(-c5ccccc5)cc4)c4ccccc4-c4ccccc4)cccc32)cc1 ; Cc1cccc(-c2ccc(C)cc2-n2c3ccccc3c3c(N(c4ccc(-c5ccccc5)cc4)c4ccccc4-c4ccccc4)cccc32)c1 ; Cc1ccc(-c2cc(C)ccc2-n2c3ccccc3c3c(N(c4ccc(-c5ccccc5)cc4)c4ccccc4-c4ccccc4)cccc32)cc1]

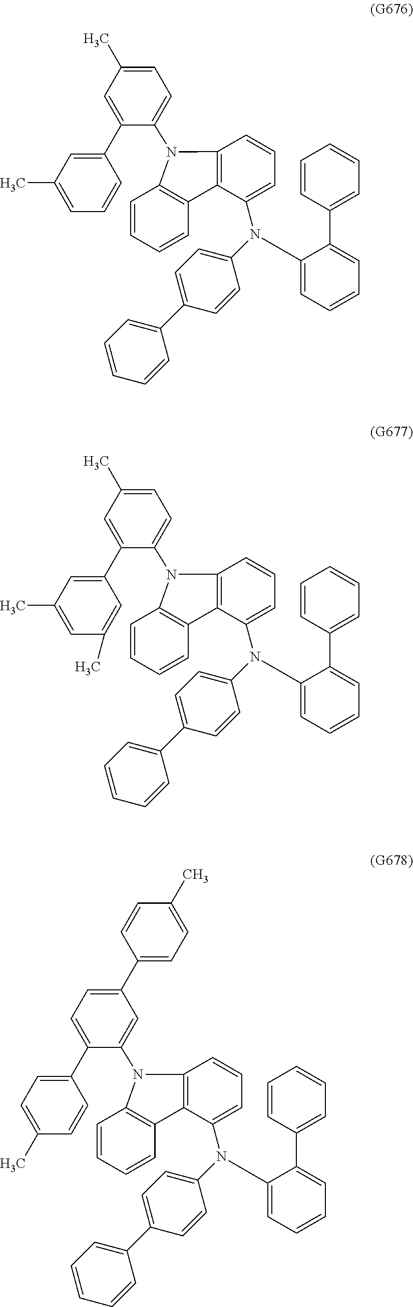 [SMILES: Cc1cccc(-c2cc(C)ccc2-n2c3ccccc3c3c(N(c4ccc(-c5ccccc5)cc4)c4ccccc4-c4ccccc4)cccc32)c1 ; Cc1cc(C)cc(-c2cc(C)ccc2-n2c3ccccc3c3c(N(c4ccc(-c5ccccc5)cc4)c4ccccc4-c4ccccc4)cccc32)c1 ; Cc1ccc(-c2ccc(-c3ccc(C)cc3)c(-n3c4ccccc4c4c(N(c5ccc(-c6ccccc6)cc5)c5ccccc5-c5ccccc5)cccc43)c2)cc1]

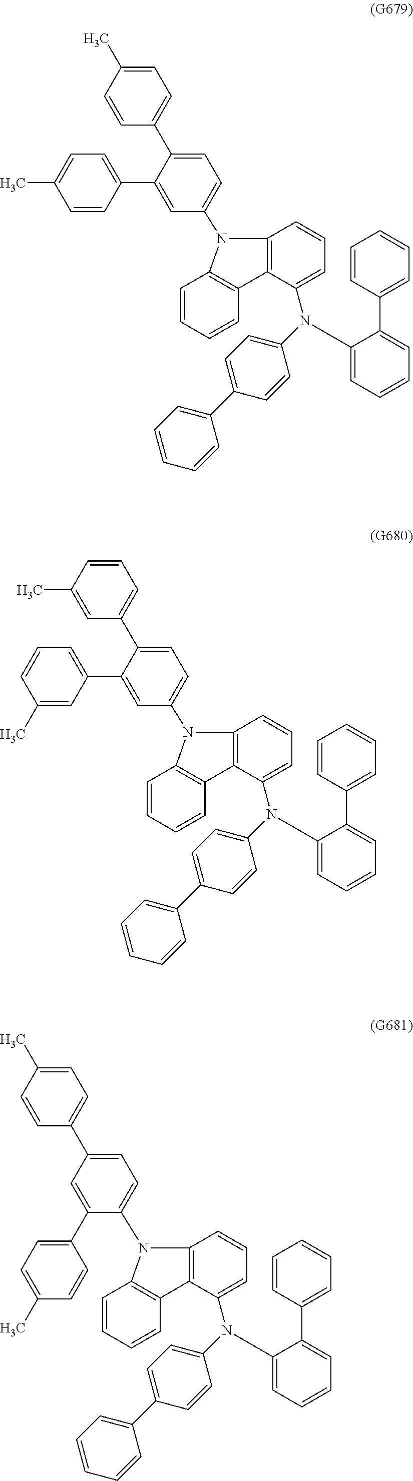 [SMILES: Cc1ccc(-c2ccc(-n3c4ccccc4c4c(N(c5ccc(-c6ccccc6)cc5)c5ccccc5-c5ccccc5)cccc43)cc2-c2ccc(C)cc2)cc1 ; Cc1cccc(-c2ccc(-n3c4ccccc4c4c(N(c5ccc(-c6ccccc6)cc5)c5ccccc5-c5ccccc5)cccc43)cc2-c2cccc(C)c2)c1 ; Cc1ccc(-c2ccc(-n3c4ccccc4c4c(N(c5ccc(-c6ccccc6)cc5)c5ccccc5-c5ccccc5)cccc43)c(-c3ccc(C)cc3)c2)cc1]

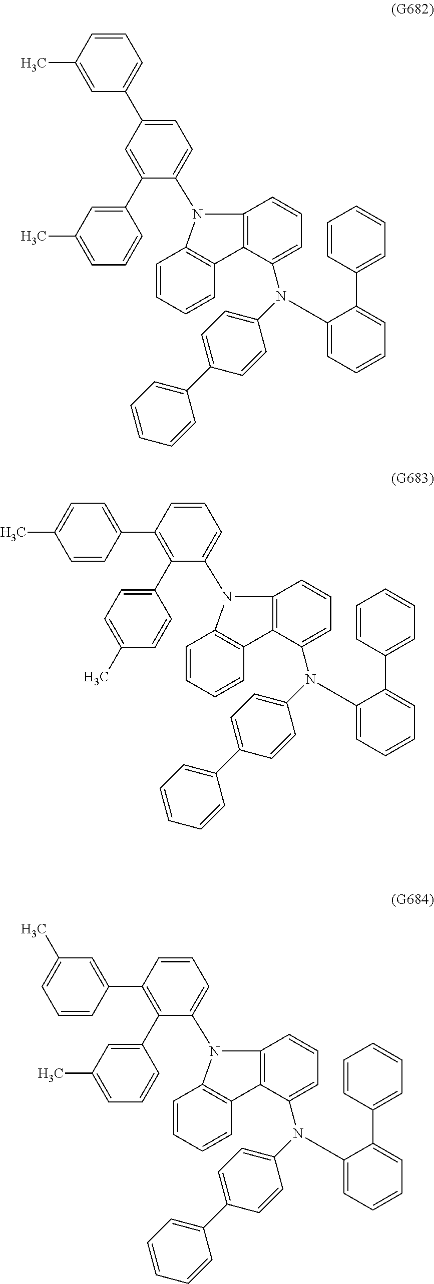 [SMILES: Cc1cccc(-c2ccc(-n3c4ccccc4c4c(N(c5ccc(-c6ccccc6)cc5)c5ccccc5-c5ccccc5)cccc43)c(-c3cccc(C)c3)c2)c1 ; Cc1ccc(-c2cccc(-n3c4ccccc4c4c(N(c5ccc(-c6ccccc6)cc5)c5ccccc5-c5ccccc5)cccc43)c2-c2ccc(C)cc2)cc1 ; Cc1cccc(-c2cccc(-n3c4ccccc4c4c(N(c5ccc(-c6ccccc6)cc5)c5ccccc5-c5ccccc5)cccc43)c2-c2cccc(C)c2)c1]

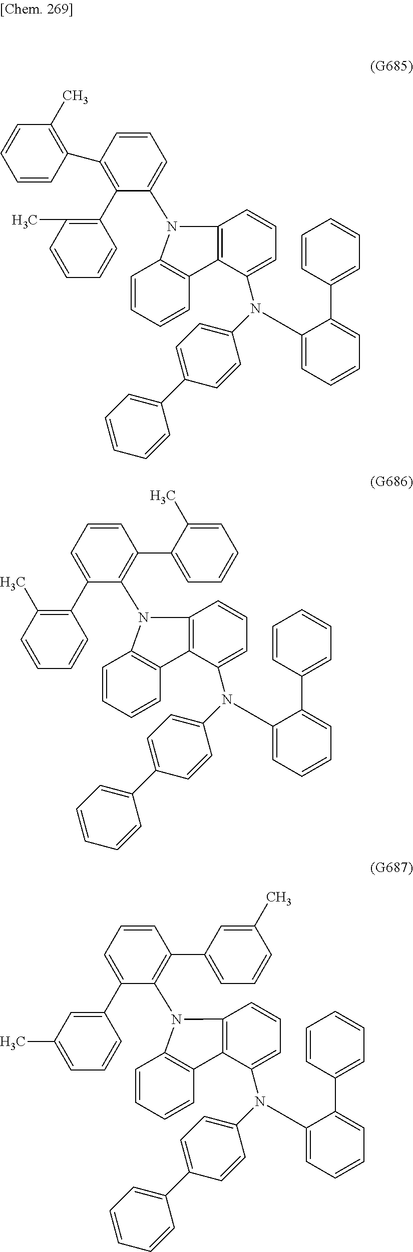

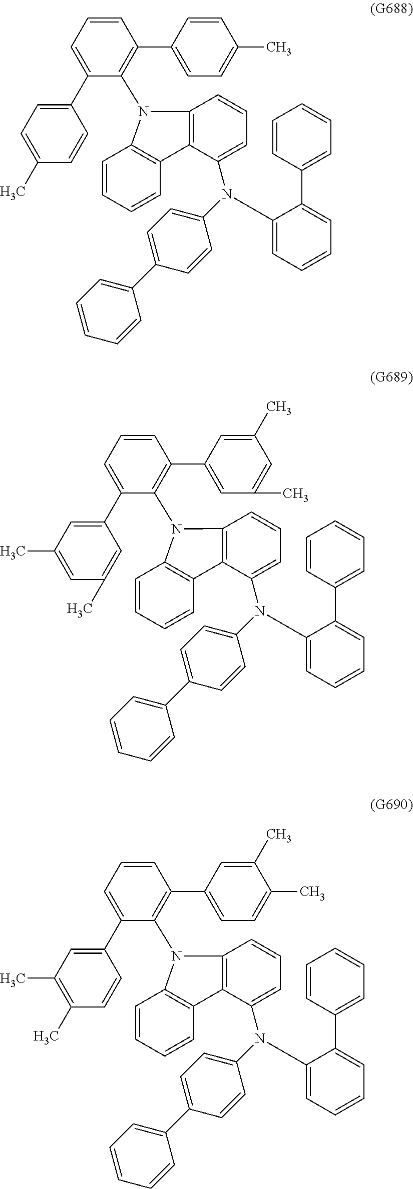 [SMILES: Cc1ccc(-c2cccc(-c3ccc(C)cc3)c2-n2c3ccccc3c3c(N(c4ccc(-c5ccccc5)cc4)c4ccccc4-c4ccccc4)cccc32)cc1 ; Cc1cc(C)cc(-c2cccc(-c3cc(C)cc(C)c3)c2-n2c3ccccc3c3c(N(c4ccc(-c5ccccc5)cc4)c4ccccc4-c4ccccc4)cccc32)c1 ; Cc1ccc(-c2cccc(-c3ccc(C)c(C)c3)c2-n2c3ccccc3c3c(N(c4ccc(-c5ccccc5)cc4)c4ccccc4-c4ccccc4)cccc32)cc1C]

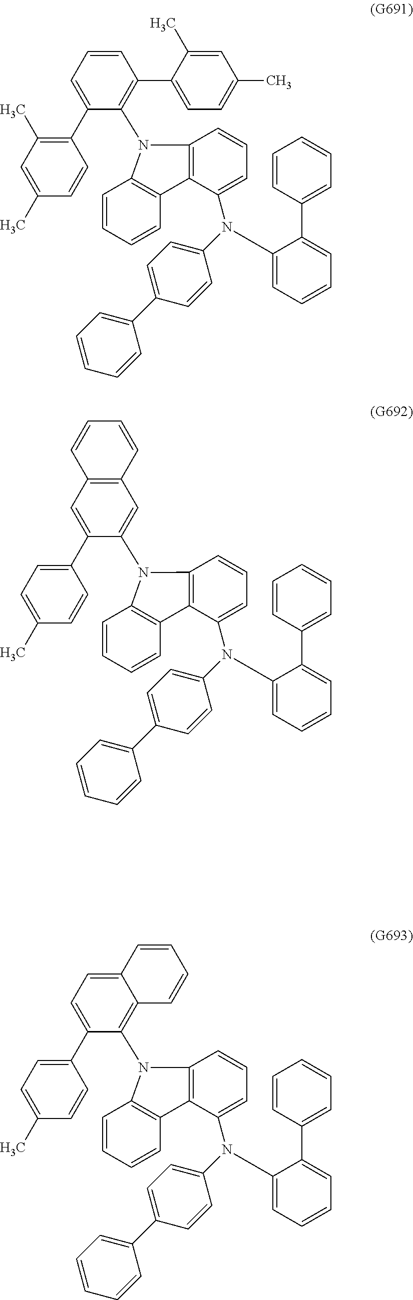

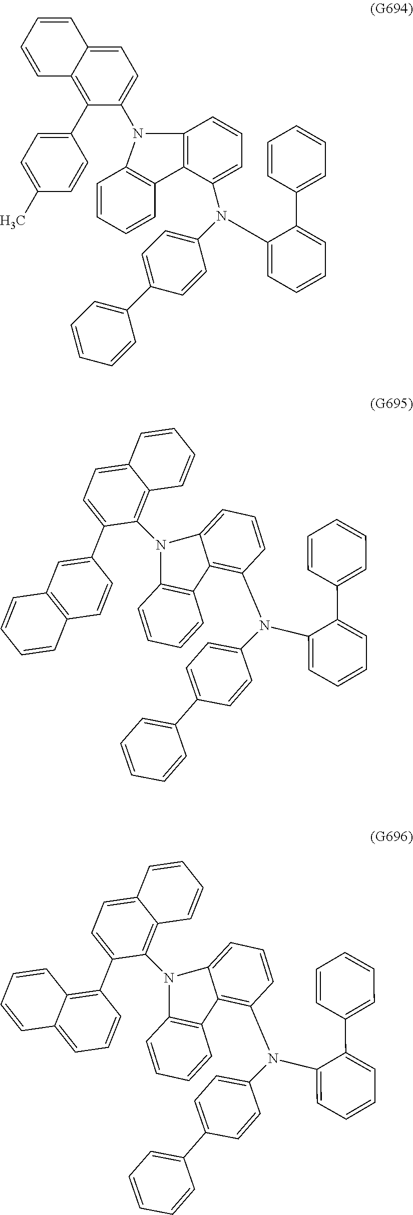 [SMILES: Cc1ccc(-c2c(-n3c4ccccc4c4c(N(c5ccc(-c6ccccc6)cc5)c5ccccc5-c5ccccc5)cccc43)ccc3ccccc23)cc1 ; c1ccc(-c2ccc(N(c3ccccc3-c3ccccc3)c3cccc4c3c3ccccc3n4-c3c(-c4ccc5ccccc5c4)ccc4ccccc34)cc2)cc1 ; c1ccc(-c2ccc(N(c3ccccc3-c3ccccc3)c3cccc4c3c3ccccc3n4-c3c(-c4cccc5ccccc45)ccc4ccccc34)cc2)cc1]

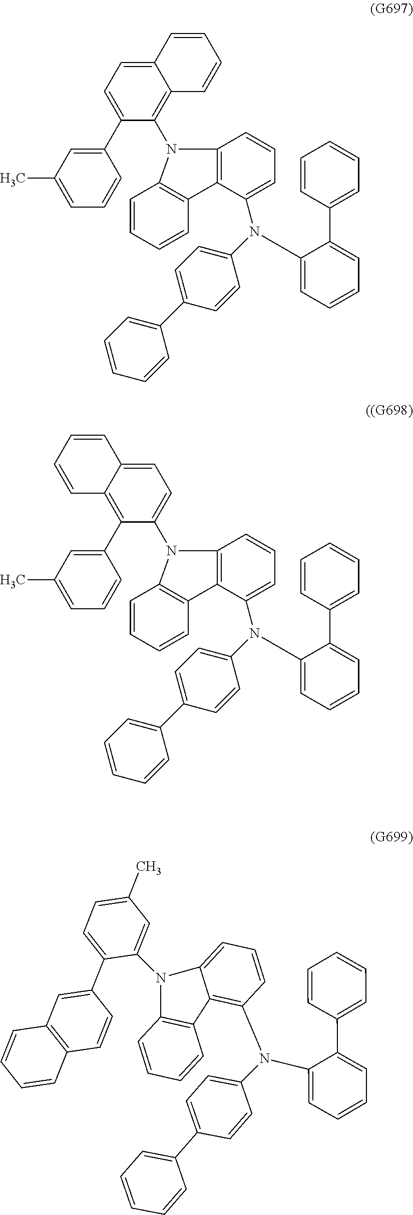 [SMILES: Cc1cccc(-c2ccc3ccccc3c2-n2c3ccccc3c3c(N(c4ccc(-c5ccccc5)cc4)c4ccccc4-c4ccccc4)cccc32)c1 ; Cc1cccc(-c2c(-n3c4ccccc4c4c(N(c5ccc(-c6ccccc6)cc5)c5ccccc5-c5ccccc5)cccc43)ccc3ccccc23)c1 ; Cc1ccc(-c2ccc3ccccc3c2)c(-n2c3ccccc3c3c(N(c4ccc(-c5ccccc5)cc4)c4ccccc4-c4ccccc4)cccc32)c1]

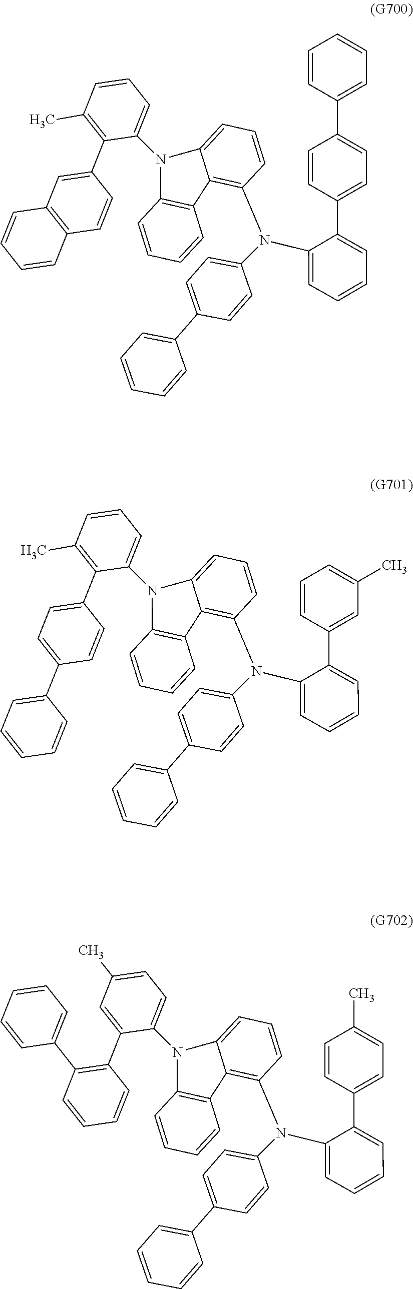 [SMILES: Cc1cccc(-n2c3ccccc3c3c(N(c4ccc(-c5ccccc5)cc4)c4ccccc4-c4ccc(-c5ccccc5)cc4)cccc32)c1-c1ccc2ccccc2c1 ; Cc1cccc(-c2ccccc2N(c2ccc(-c3ccccc3)cc2)c2cccc3c2c2ccccc2n3-c2cccc(C)c2-c2ccc(-c3ccccc3)cc2)c1 ; Cc1ccc(-c2ccccc2N(c2ccc(-c3ccccc3)cc2)c2cccc3c2c2ccccc2n3-c2ccc(C)cc2-c2ccccc2-c2ccccc2)cc1]

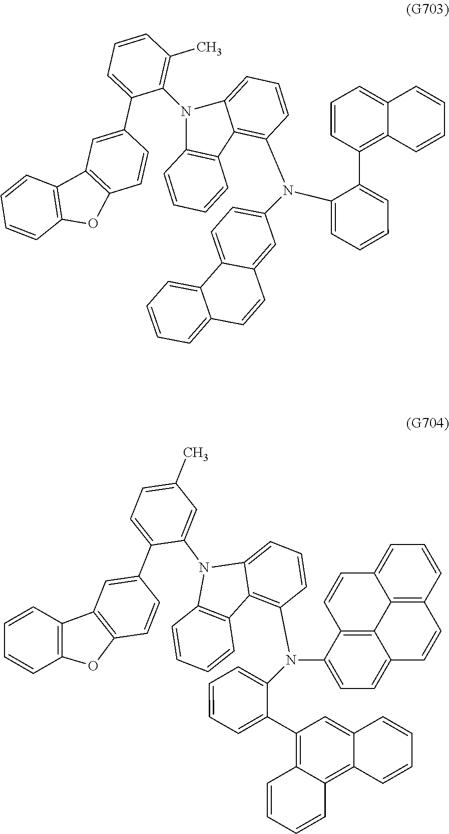 [SMILES: Cc1cccc(-c2ccc3oc4ccccc4c3c2)c1-n1c2ccccc2c2c(N(c3ccc4c(ccc5ccccc54)c3)c3ccccc3-c3cccc4ccccc34)cccc21 ; Cc1ccc(-c2ccc3oc4ccccc4c3c2)c(-n2c3ccccc3c3c(N(c4ccccc4-c4cc5ccccc5c5ccccc45)c4ccc5ccc6cccc7ccc4c5c67)cccc32)c1]

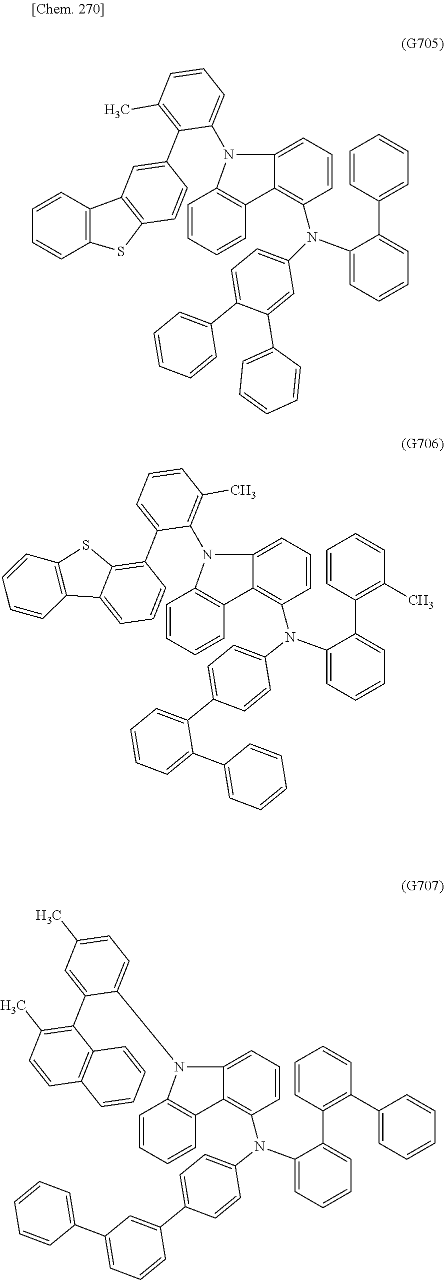 [SMILES: Cc1cccc(-n2c3ccccc3c3c(N(c4ccc(-c5ccccc5)c(-c5ccccc5)c4)c4ccccc4-c4ccccc4)cccc32)c1-c1ccc2sc3ccccc3c2c1 ; Cc1ccccc1-c1ccccc1N(c1ccc(-c2ccccc2-c2ccccc2)cc1)c1cccc2c1c1ccccc1n2-c1c(C)cccc1-c1cccc2c1sc1ccccc12 ; Cc1ccc(-n2c3ccccc3c3c(N(c4ccc(-c5cccc(-c6ccccc6)c5)cc4)c4ccccc4-c4ccccc4-c4ccccc4)cccc32)c(-c2c(C)ccc3ccccc23)c1]

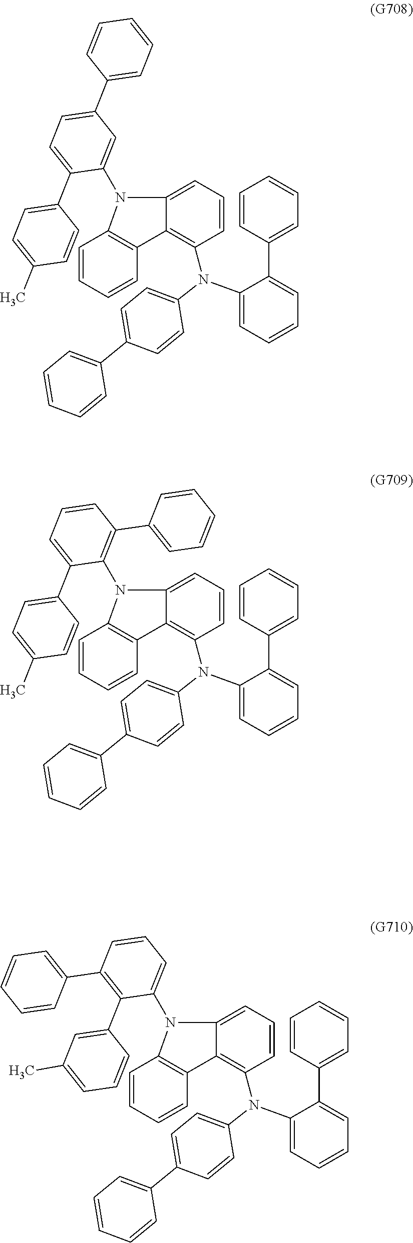 [SMILES: Cc1ccc(-c2ccc(-c3ccccc3)cc2-n2c3ccccc3c3c(N(c4ccc(-c5ccccc5)cc4)c4ccccc4-c4ccccc4)cccc32)cc1 ; Cc1ccc(-c2cccc(-c3ccccc3)c2-n2c3ccccc3c3c(N(c4ccc(-c5ccccc5)cc4)c4ccccc4-c4ccccc4)cccc32)cc1 ; Cc1cccc(-c2c(-c3ccccc3)cccc2-n2c3ccccc3c3c(N(c4ccc(-c5ccccc5)cc4)c4ccccc4-c4ccccc4)cccc32)c1]

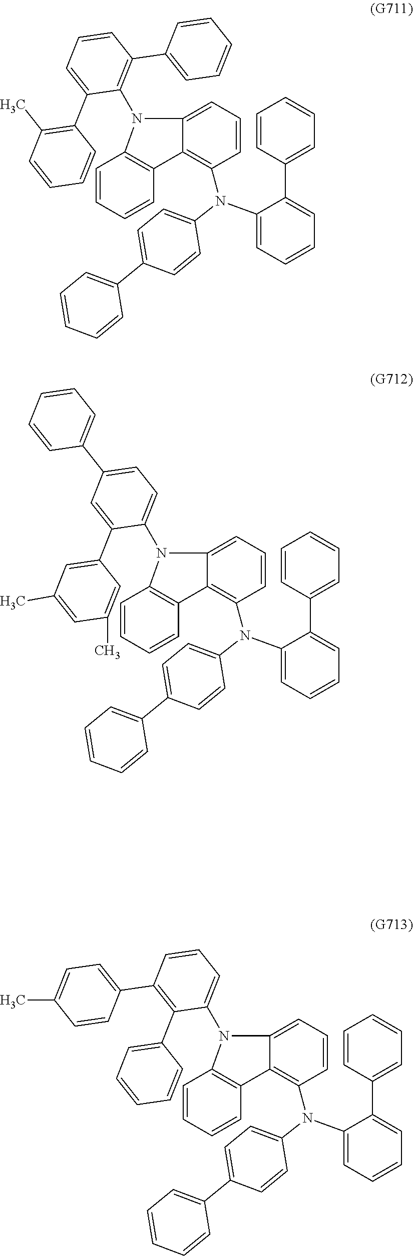 [SMILES: Cc1ccccc1-c1cccc(-c2ccccc2)c1-n1c2ccccc2c2c(N(c3ccc(-c4ccccc4)cc3)c3ccccc3-c3ccccc3)cccc21 ; Cc1cc(C)cc(-c2cc(-c3ccccc3)ccc2-n2c3ccccc3c3c(N(c4ccc(-c5ccccc5)cc4)c4ccccc4-c4ccccc4)cccc32)c1 ; Cc1ccc(-c2cccc(-n3c4ccccc4c4c(N(c5ccc(-c6ccccc6)cc5)c5ccccc5-c5ccccc5)cccc43)c2-c2ccccc2)cc1]

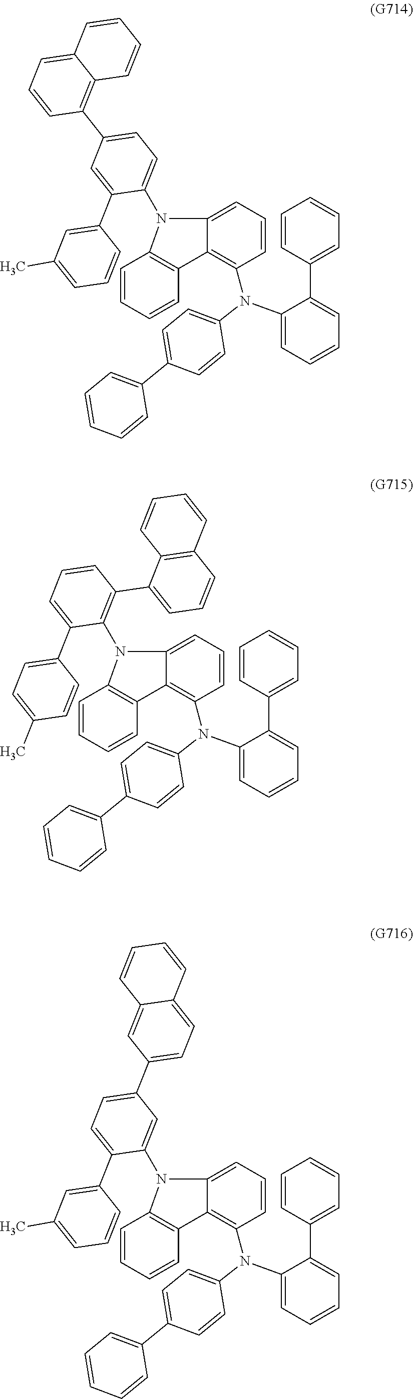 [SMILES: Cc1cccc(-c2cc(-c3cccc4ccccc34)ccc2-n2c3ccccc3c3c(N(c4ccc(-c5ccccc5)cc4)c4ccccc4-c4ccccc4)cccc32)c1 ; Cc1ccc(-c2cccc(-c3cccc4ccccc34)c2-n2c3ccccc3c3c(N(c4ccc(-c5ccccc5)cc4)c4ccccc4-c4ccccc4)cccc32)cc1 ; Cc1cccc(-c2ccc(-c3ccc4ccccc4c3)cc2-n2c3ccccc3c3c(N(c4ccc(-c5ccccc5)cc4)c4ccccc4-c4ccccc4)cccc32)c1]

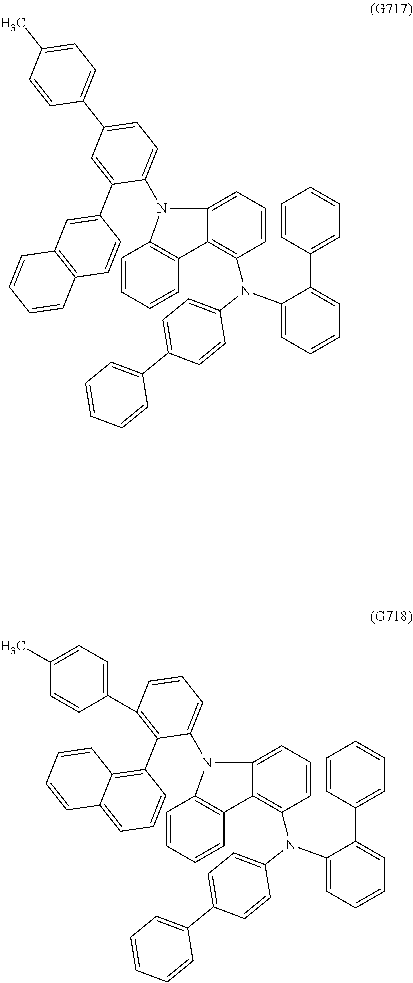 [SMILES: Cc1ccc(-c2ccc(-n3c4ccccc4c4c(N(c5ccc(-c6ccccc6)cc5)c5ccccc5-c5ccccc5)cccc43)c(-c3ccc4ccccc4c3)c2)cc1 ; Cc1ccc(-c2cccc(-n3c4ccccc4c4c(N(c5ccc(-c6ccccc6)cc5)c5ccccc5-c5ccccc5)cccc43)c2-c2cccc3ccccc23)cc1]

### [Organic Electroluminescent Element and Hole-Transport Layer]

A transverse current suppressing material represented by the formula (1) (hereinafter sometimes referred to simply as a transverse current blocking material (1)) or an organic electroluminescent element containing a carbazole compound represented by the formula (22) or (23) (hereinafter sometimes referred to simply as an organic electroluminescent element) is described below.

An organic electroluminescent element according to an embodiment of the present disclosure contains a transverse current suppressing material represented by the formula (1) or a carbazole compound represented by the formula (22) or (23).

The configuration of an organic electroluminescent element is, for example, but not limited to, one of the configurations (i) to (v):
(i): Positive electrode/hole-injection layer/light-emitting layer/negative electrode
(ii): Positive electrode/hole-injection layer/hole-transport layer/light-emitting layer/negative electrode
(iii): Positive electrode/hole-injection layer/hole-transport layer/electron-blocking layer/light-emitting layer/negative electrode
(iv): Positive electrode/hole-injection layer/hole-transport layer/electron-blocking layer/light-emitting layer/electron-transport layer/negative electrode
(v): Positive electrode/hole-injection layer/hole-transport layer/electron-blocking layer/light-emitting layer/electron-transport layer/electron-injection layer/negative electrode

A transverse current suppressing material represented by the formula (1) or a carbazole compound represented by the formula (22) or (23) can suppress the transverse current of an organic electroluminescent element. A hole-injection layer contains a transverse current suppressing material represented by the formula (1) or a carbazole compound represented by the formula (22) or (23). In an organic electroluminescent element including a hole-transport layer, the hole-transport layer may contain a transverse current suppressing material represented by the formula (1) or a carbazole compound represented by the formula (22) or (23).

Preferably, an organic electroluminescent element comprising:
a positive electrode;
a plurality of organic layers on the positive electrode; and
a negative electrode on the plurality of organic layers,
one or more layers of the plurality of organic layers contain a carbazole compound represented by the formula (22) or (23).

At least one layer of a hole-injection layer, a hole-transport layer, an electron-blocking layer and a light-emitting layer preferably contains a carbazole compound represented by the formula (22) or (23) in terms of good emission properties, drive voltage and life of an organic electroluminescent element.

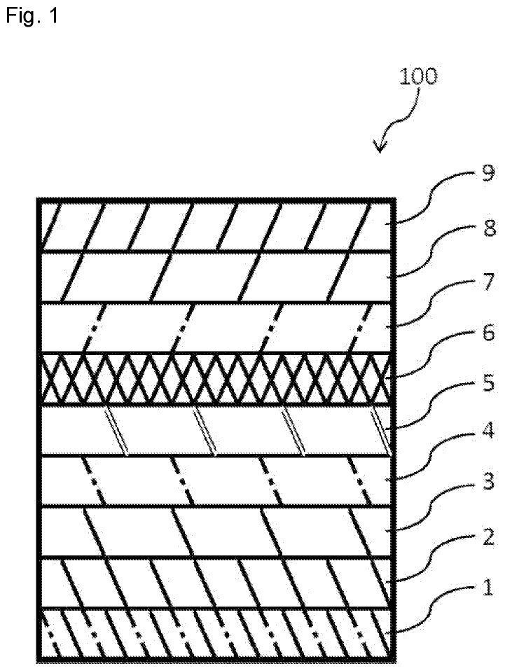

An organic electroluminescent element according to an embodiment of the present disclosure is described in more detail below with reference to FIG. 1 by taking the configuration (v) as an example.

An organic electroluminescent element illustrated in FIG. 1 has a so-called bottom emission type element configuration. An organic electroluminescent element according to an embodiment of the present disclosure is not limited to the bottom emission type element configuration. More specifically, an organic electroluminescent element according to an embodiment of the present disclosure may have another known element configuration, such as a top emission type.

FIG. 1 is a schematic cross-sectional view of an example of a layered configuration of an organic electroluminescent element according to an embodiment of the present disclosure.

An organic electroluminescent element 100 includes a substrate 1, a positive electrode 2, a hole-injection layer 3, a hole-transport layer 4, an electron-blocking layer 5, a light-emitting layer 6, an electron-transport layer 7, an electron-injection layer 8 and a negative electrode 9 in this order. Some of these layers may be omitted, or on the contrary another layer may be added. For example, a hole-blocking layer may be provided between the light-emitting layer 6 and the electron-transport layer 7, or the light-emitting layer 6 may be directly provided on the hole-transport layer 4 without the electron-blocking layer 5. A single layer having the functions of a plurality of layers, such as a hole-transport/electron-blocking layer having the functions of the hole-transport layer 4 and the electron-blocking layer 5 in a single layer, may be provided instead of the plurality of layers. Furthermore, for example, the monolayer electron-transport layer 7 may be composed of a plurality of layers.

### <<Layer Containing Transverse Current Suppressing Material (1)>>

In the configuration example illustrated in FIG. 1, in the organic electroluminescent element 100, the hole-injection layer 3 or the hole-injection layer 3 and the hole-transport layer 4 contain the transverse current blocking material (1). In particular, the hole-injection layer 3 and the hole-transport layer 4 preferably contain the transverse current blocking material (1). The transverse current blocking material (1) may be contained in a plurality of layers of the organic electroluminescent element.

In the organic electroluminescent element 100 described below, the hole-injection layer 3 and the hole-transport layer 4 contain the transverse current suppressing material (1).

### <Substrate 1>

The substrate 1 is, for example, but not limited to, a glass sheet, a quartz sheet, a plastic sheet, or the like.

The substrate 1 is, for example, a glass sheet, a quartz sheet, a plastic sheet, a plastic film, or the like. Among these, a glass sheet, a quartz sheet and a transparent or translucent plastic film are preferred.

The transparent or translucent plastic film is, for example, a film made of poly(ethylene terephthalate) (PET), poly(ethylene naphthalate) (PEN), poly(ether sulfone) (PES), poly(ether imide), poly(ether ether ketone), poly(phenylene sulfide), polyarylate, polyimide, polycarbonate (PC), cellulose triacetate (TAC), cellulose acetate propionate (CAP) or the like.

When light emission is extracted from the substrate 1, the substrate 1 is transparent to the wavelength of light.

### <Positive Electrode 2>

The positive electrode 2 is provided on the substrate 1 (on the hole-injection layer 3 side).

A material of the positive electrode may be a metal, an alloy or an electrically conductive compound each having a high work function (for example, 4 eV or more) or a mixture thereof. Specific examples of the material of the positive electrode include metals, such as Au, and electrically conductive transparent materials, such as CuI, indium tin oxide (ITO), SnO₂ and ZnO.

In an organic electroluminescent element in which light emission is extracted through a positive electrode, the positive electrode is formed of an electrically conductive transparent material that transmits or substantially transmits the light emission.

### <Hole-Injection Layer 3>

The hole-injection layer 3 is provided between the positive electrode 2 and the hole-transport layer 4 described later.

The hole-injection layer functions as a hole-injecting layer. The hole-injection layer between the positive electrode and the light-emitting layer allows a hole to be injected into the light-emitting layer at a lower electric field. In particular, the hole-injection layer preferably contains a transverse current suppressing material represented by the formula (1) or a carbazole compound represented by the formula (22) or (23).

The hole-injection layer may further contain an electron-accepting p-dopant.

Thus, a hole-injection layer according to an embodiment of the present disclosure comprises:
a first compound; and
a second compound,
wherein the first compound is
a transverse current suppressing material represented by the formula (1) or
a carbazole compound represented by the formula (22) or (23), and
the second compound is an electron-accepting p-dopant.

Preferably, a third compound is further contained, and
the third compound is a hole-transporting triarylamine compound.

In these cases, the p-dopant content is 0.5% by mass or more and 20% by mass or less. A transverse current suppressing material represented by the formula (1) or a carbazole compound represented by the formula (22) or (23) constitutes 20% by mass or more and 99.5% by mass or less.

The p-dopant may have an electron acceptor property and is, for example, a compound represented by one of the formulae (J1) to (J51):

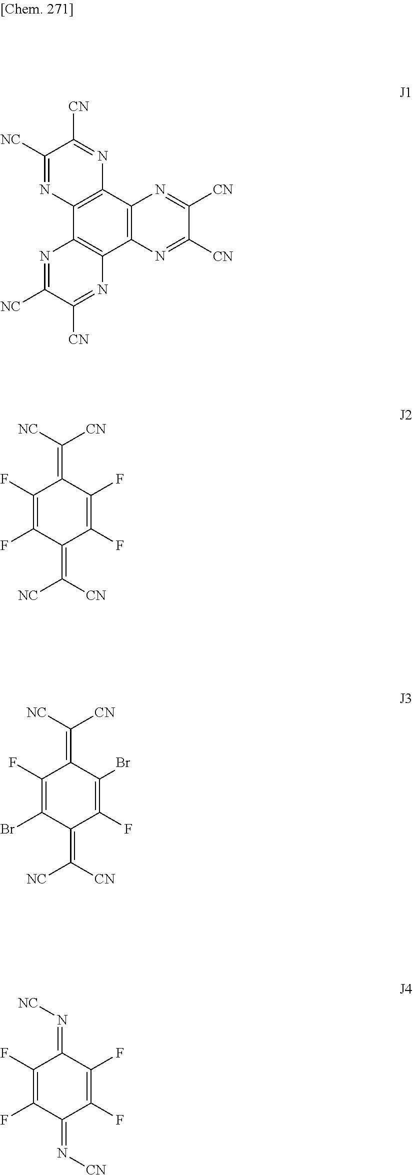 [SMILES: N#Cc1nc2c3nc(C#N)c(C#N)nc3c3nc(C#N)c(C#N)nc3c2nc1C#N ; N#CC(C#N)=c1c(F)c(F)c(=C(C#N)C#N)c(F)c1F ; N#CC(C#N)=c1c(F)c(Br)c(=C(C#N)C#N)c(F)c1Br ; N#CN=C1C(F)=C(F)C(=NC#N)C(F)=C1F]

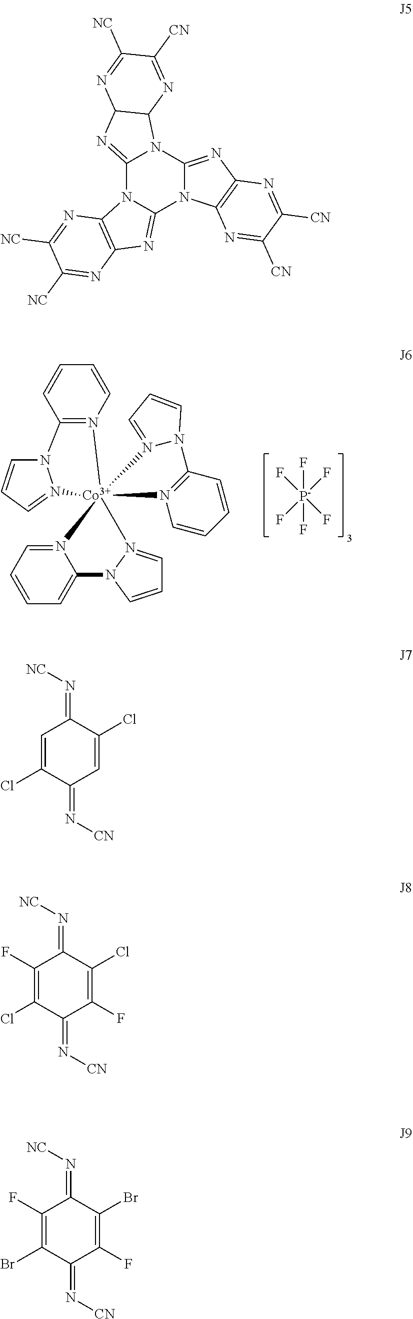

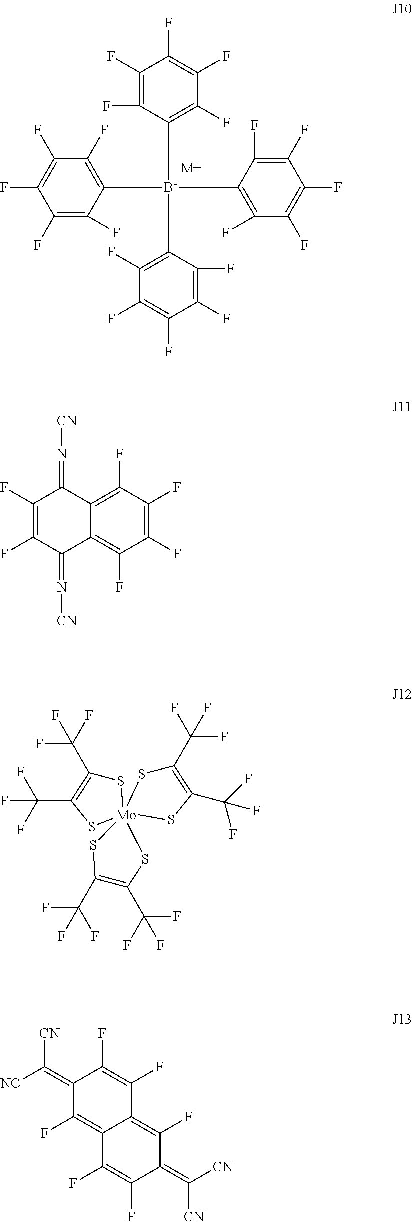

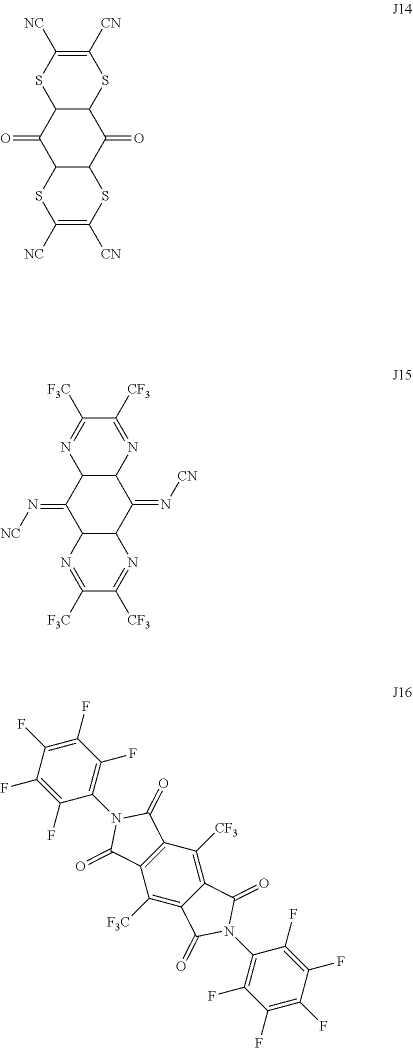 [SMILES: N#CC1=C(C#N)SC2C(=O)C3SC(C#N)=C(C#N)SC3C(=O)C2S1 ; N#CN=C1C2N=C(C(F)(F)F)C(C(F)(F)F)=NC2C(=NC#N)C2N=C(C(F)(F)F)C(C(F)(F)F)=NC12 ; O=c1c2c(C(F)(F)F)c3c(=O)n(-c4c(F)c(F)c(F)c(F)c4F)c(=O)c3c(C(F)(F)F)c2c(=O)n1-c1c(F)c(F)c(F)c(F)c1F]

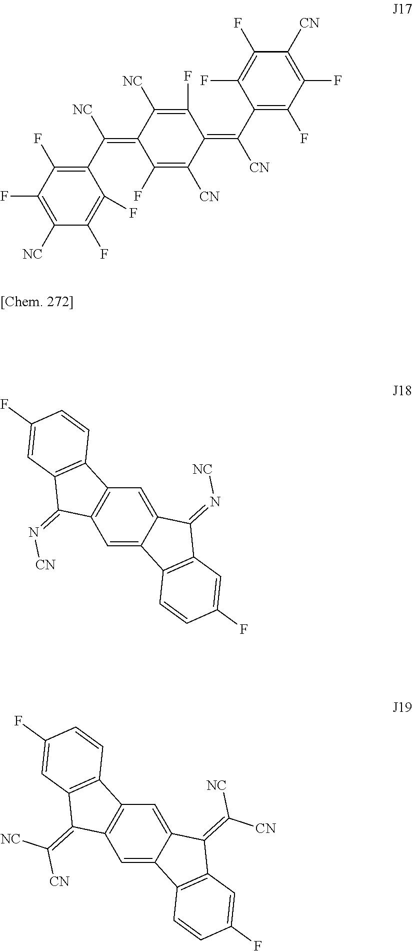 [SMILES: N#C/C(c1c(F)c(F)c(C#N)c(F)c1F)=c1/c(F)c(C#N)/c(=C(\C#N)c2c(F)c(F)c(C#N)c(F)c2F)c(F)c1C#N ; N#C/N=c1/c2cc(F)ccc2c2cc3/c(=N\C#N)c4cc(F)ccc4c3cc12 ; N#CC(C#N)=C1c2cc(F)ccc2-c2cc3c(cc21)-c1ccc(F)cc1C3=C(C#N)C#N]

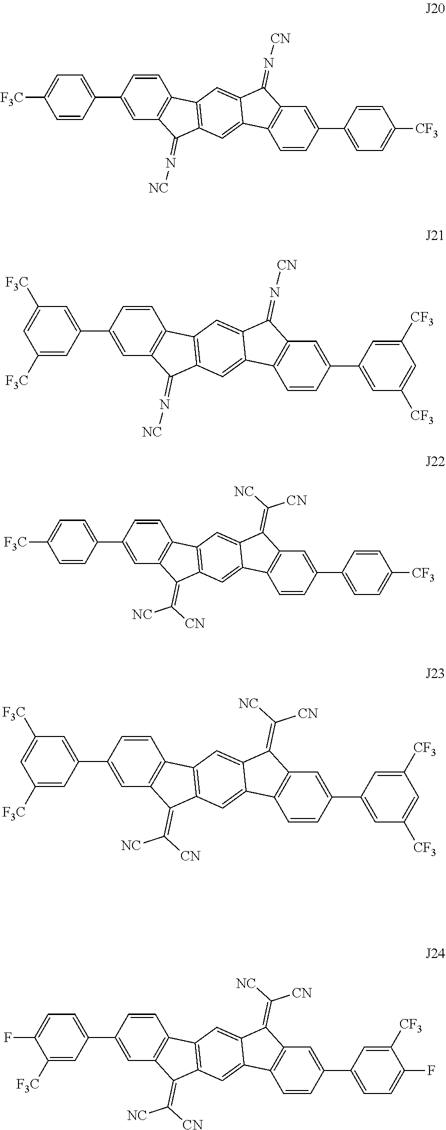

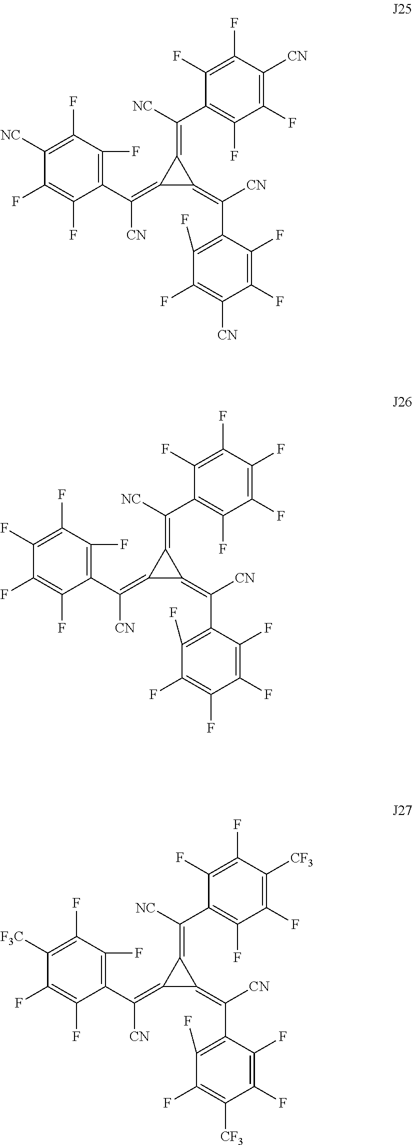 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F ; N#CC(=C1C(=C(C#N)c2c(F)c(F)c(F)c(F)c2F)C1=C(C#N)c1c(F)c(F)c(F)c(F)c1F)c1c(F)c(F)c(F)c(F)c1F ; N#CC(=C1C(=C(C#N)c2c(F)c(F)c(C(F)(F)F)c(F)c2F)C1=C(C#N)c1c(F)c(F)c(C(F)(F)F)c(F)c1F)c1c(F)c(F)c(C(F)(F)F)c(F)c1F]

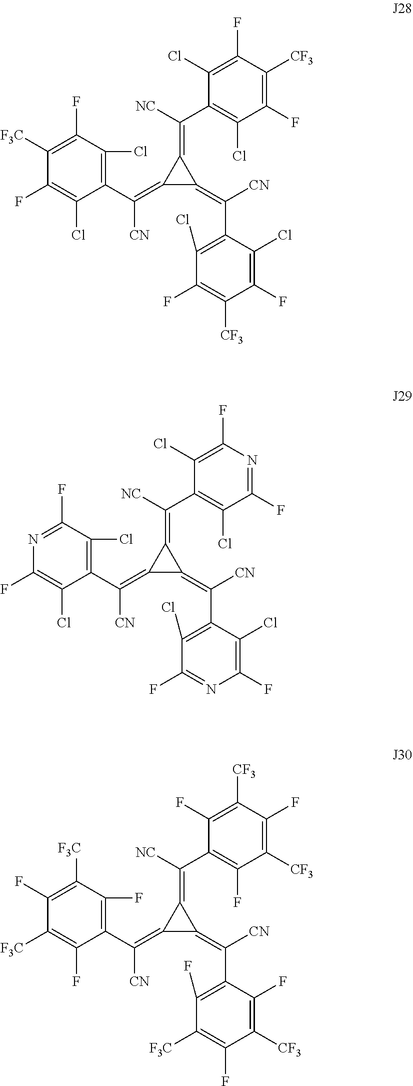 [SMILES: N#CC(=C1C(=C(C#N)c2c(Cl)c(F)c(C(F)(F)F)c(F)c2Cl)C1=C(C#N)c1c(Cl)c(F)c(C(F)(F)F)c(F)c1Cl)c1c(Cl)c(F)c(C(F)(F)F)c(F)c1Cl ; N#CC(=C1C(=C(C#N)c2c(Cl)c(F)nc(F)c2Cl)C1=C(C#N)c1c(Cl)c(F)nc(F)c1Cl)c1c(Cl)c(F)nc(F)c1Cl ; N#CC(=C1C(=C(C#N)c2c(F)c(C(F)(F)F)c(F)c(C(F)(F)F)c2F)C1=C(C#N)c1c(F)c(C(F)(F)F)c(F)c(C(F)(F)F)c1F)c1c(F)c(C(F)(F)F)c(F)c(C(F)(F)F)c1F]

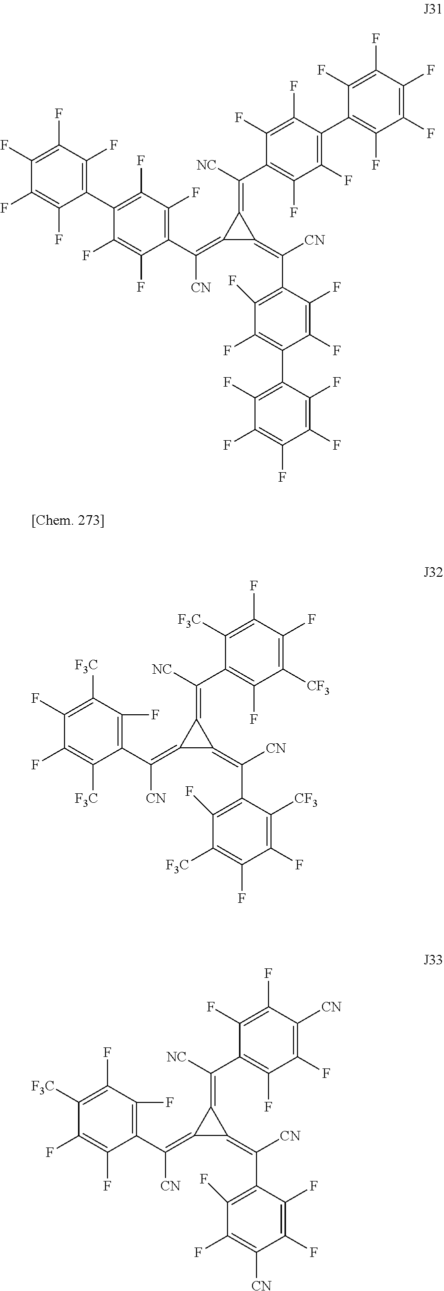 [SMILES: N#CC(=C1C(=C(C#N)c2c(F)c(F)c(-c3c(F)c(F)c(F)c(F)c3F)c(F)c2F)C1=C(C#N)c1c(F)c(F)c(-c2c(F)c(F)c(F)c(F)c2F)c(F)c1F)c1c(F)c(F)c(-c2c(F)c(F)c(F)c(F)c2F)c(F)c1F ; N#CC(=C1C(=C(C#N)c2c(F)c(C(F)(F)F)c(F)c(F)c2C(F)(F)F)C1=C(C#N)c1c(F)c(C(F)(F)F)c(F)c(F)c1C(F)(F)F)c1c(F)c(C(F)(F)F)c(F)c(F)c1C(F)(F)F ; N#C/C(=C1/C(=C(\C#N)c2c(F)c(F)c(C#N)c(F)c2F)/C1=C(\C#N)c1c(F)c(F)c(C(F)(F)F)c(F)c1F)c1c(F)c(F)c(C#N)c(F)c1F]

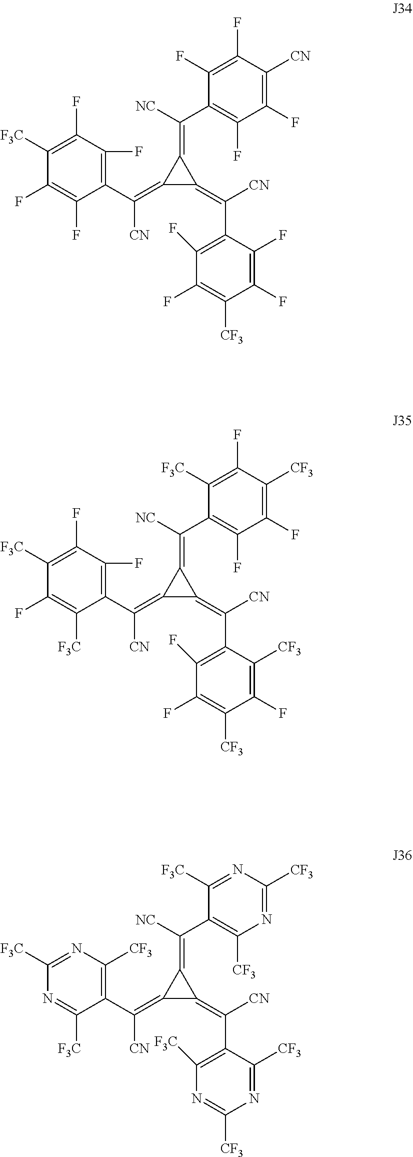 [SMILES: N#C/C(=C1\C(=C(\C#N)c2c(F)c(F)c(C(F)(F)F)c(F)c2F)\C1=C(\C#N)c1c(F)c(F)c(C(F)(F)F)c(F)c1F)c1c(F)c(F)c(C#N)c(F)c1F ; N#CC(=C1C(=C(C#N)c2c(F)c(F)c(C(F)(F)F)c(F)c2C(F)(F)F)C1=C(C#N)c1c(F)c(F)c(C(F)(F)F)c(F)c1C(F)(F)F)c1c(F)c(F)c(C(F)(F)F)c(F)c1C(F)(F)F ; N#CC(=C1C(=C(C#N)c2c(C(F)(F)F)nc(C(F)(F)F)nc2C(F)(F)F)C1=C(C#N)c1c(C(F)(F)F)nc(C(F)(F)F)nc1C(F)(F)F)c1c(C(F)(F)F)nc(C(F)(F)F)nc1C(F)(F)F]

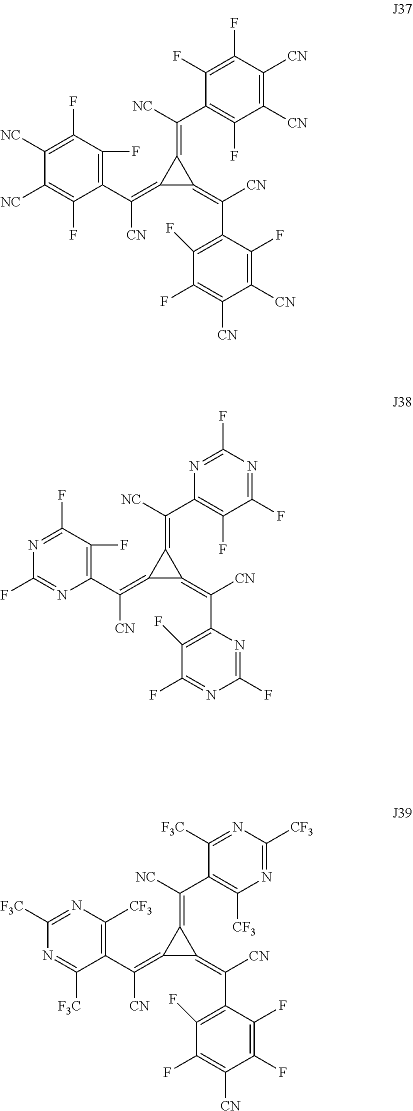 [SMILES: N#CC(=C1C(=C(C#N)c2c(F)c(F)c(C#N)c(C#N)c2F)C1=C(C#N)c1c(F)c(F)c(C#N)c(C#N)c1F)c1c(F)c(F)c(C#N)c(C#N)c1F ; N#CC(=C1C(=C(C#N)c2nc(F)nc(F)c2F)C1=C(C#N)c1nc(F)nc(F)c1F)c1nc(F)nc(F)c1F ; N#C/C(=C1/C(=C(\C#N)c2c(C(F)(F)F)nc(C(F)(F)F)nc2C(F)(F)F)/C1=C(\C#N)c1c(F)c(F)c(C#N)c(F)c1F)c1c(C(F)(F)F)nc(C(F)(F)F)nc1C(F)(F)F]

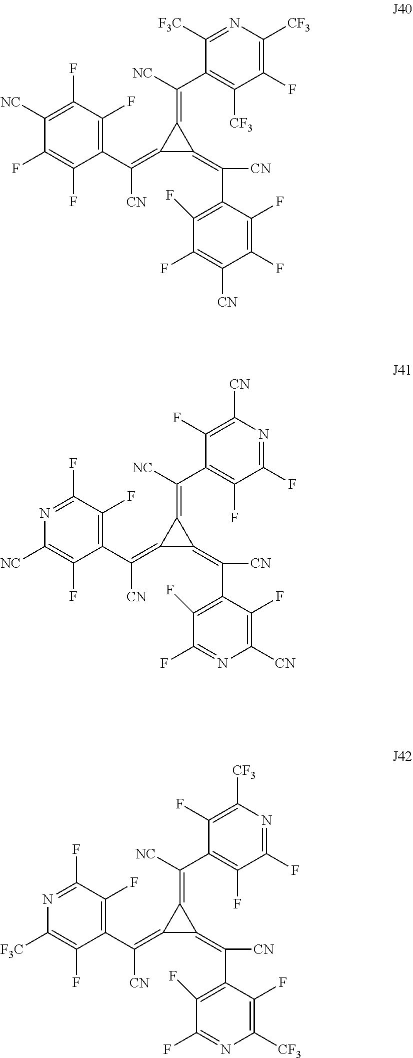 [SMILES: N#C/C(=C1/C(=C(\C#N)c2c(C(F)(F)F)nc(C(F)(F)F)c(F)c2C(F)(F)F)/C1=C(\C#N)c1c(F)c(F)c(C#N)c(F)c1F)c1c(F)c(F)c(C#N)c(F)c1F ; N#CC(=C1C(=C(C#N)c2c(F)c(F)nc(C#N)c2F)C1=C(C#N)c1c(F)c(F)nc(C#N)c1F)c1c(F)c(F)nc(C#N)c1F ; N#CC(=C1C(=C(C#N)c2c(F)c(F)nc(C(F)(F)F)c2F)C1=C(C#N)c1c(F)c(F)nc(C(F)(F)F)c1F)c1c(F)c(F)nc(C(F)(F)F)c1F]

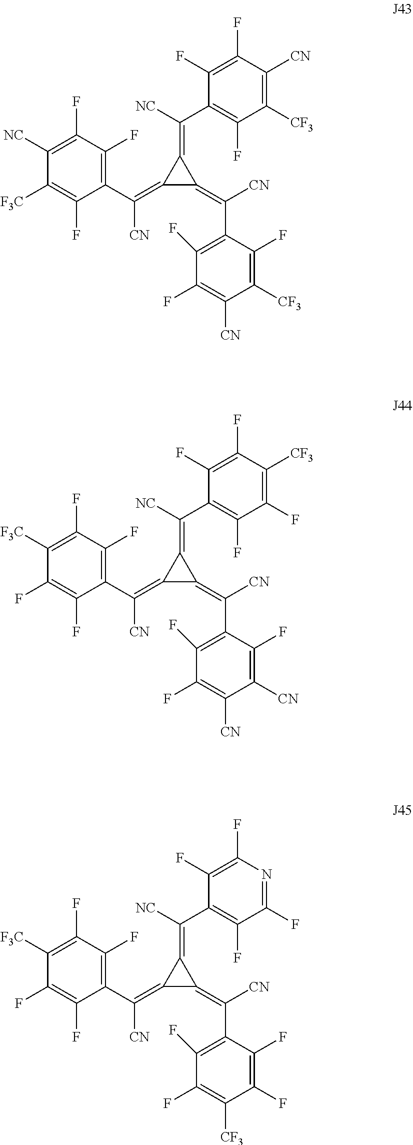 [SMILES: N#CC(=C1C(=C(C#N)c2c(F)c(F)c(C#N)c(C(F)(F)F)c2F)C1=C(C#N)c1c(F)c(F)c(C#N)c(C(F)(F)F)c1F)c1c(F)c(F)c(C#N)c(C(F)(F)F)c1F ; N#C/C(=C1/C(=C(\C#N)c2c(F)c(F)c(C#N)c(C#N)c2F)/C1=C(\C#N)c1c(F)c(F)c(C(F)(F)F)c(F)c1F)c1c(F)c(F)c(C(F)(F)F)c(F)c1F ; N#C/C(=C1\C(=C(\C#N)c2c(F)c(F)nc(F)c2F)\C1=C(\C#N)c1c(F)c(F)c(C(F)(F)F)c(F)c1F)c1c(F)c(F)c(C(F)(F)F)c(F)c1F]

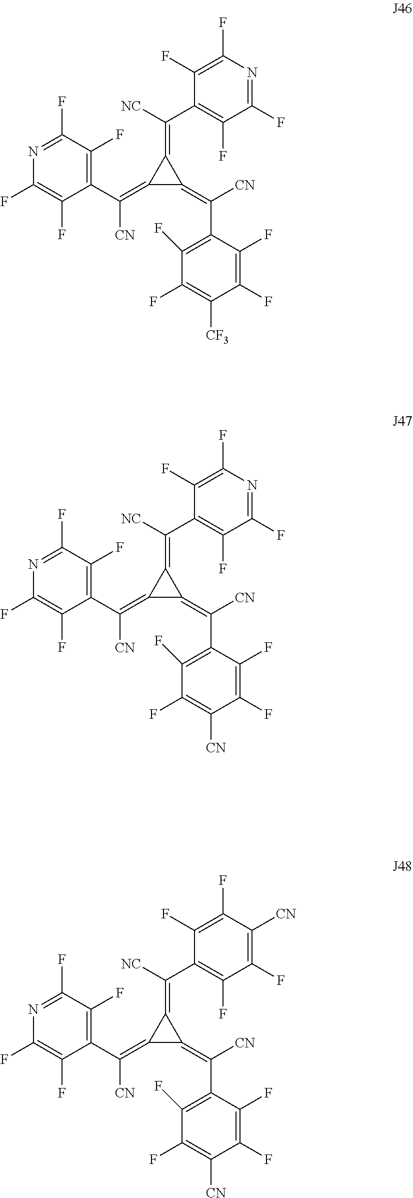 [SMILES: N#C/C(=C1/C(=C(\C#N)c2c(F)c(F)c(C(F)(F)F)c(F)c2F)/C1=C(\C#N)c1c(F)c(F)nc(F)c1F)c1c(F)c(F)nc(F)c1F ; N#C/C(=C1/C(=C(\C#N)c2c(F)c(F)c(C#N)c(F)c2F)/C1=C(\C#N)c1c(F)c(F)nc(F)c1F)c1c(F)c(F)nc(F)c1F ; N#C/C(=C1\C(=C(\C#N)c2c(F)c(F)nc(F)c2F)\C1=C(\C#N)c1c(F)c(F)c(C#N)c(F)c1F)c1c(F)c(F)c(C#N)c(F)c1F]

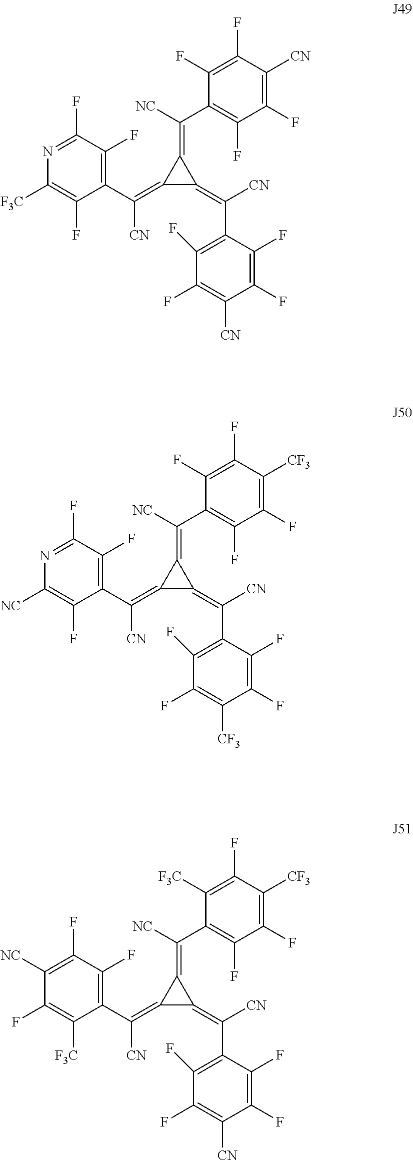 [SMILES: N#C/C(=C1\C(=C(\C#N)c2c(F)c(F)nc(C(F)(F)F)c2F)\C1=C(\C#N)c1c(F)c(F)c(C#N)c(F)c1F)c1c(F)c(F)c(C#N)c(F)c1F ; N#C/C(=C1\C(=C(\C#N)c2c(F)c(F)nc(C#N)c2F)\C1=C(\C#N)c1c(F)c(F)c(C(F)(F)F)c(F)c1F)c1c(F)c(F)c(C(F)(F)F)c(F)c1F ; N#C/C(=C1/C(=C(\C#N)c2c(F)c(F)c(C#N)c(F)c2C(F)(F)F)/C1=C(\C#N)c1c(F)c(F)c(C(F)(F)F)c(F)c1C(F)(F)F)c1c(F)c(F)c(C#N)c(F)c1F]

The hole-injection layer may further contain a hole-transporting triarylamine compound. In this case, the triarylamine compound content is 10% by mass or more and 79.5% by mass or less. The triarylamine compound is represented by any one of the formulae (36) to (38):

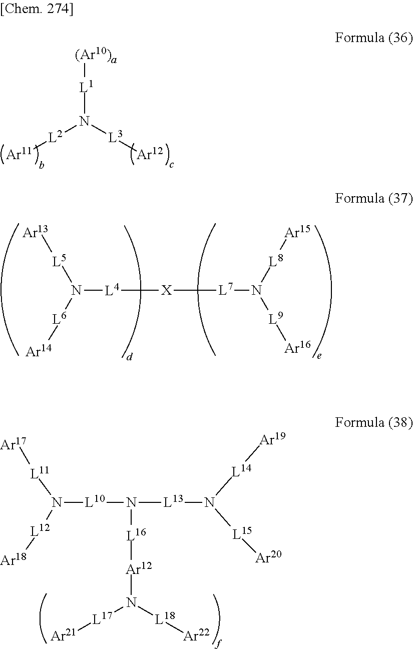

wherein
Ar¹⁰ to Ar²² each independently denote
an optionally substituted monocyclic, linked or fused aromatic hydrocarbon group with 6 to 25 carbon atoms or
an optionally substituted monocyclic, linked or fused heteroaromatic group with 3 to 25 carbon atoms;
L¹ to L¹⁸ each independently denotes
an optionally substituted monocyclic, linked or fused-ring divalent aromatic hydrocarbon group with 6 to 25 carbon atoms,
an optionally substituted monocyclic, linked or fused-ring divalent heteroaromatic group with 3 to 25 carbon atoms or
a single bond;
X denotes
an optionally substituted monocyclic, linked or fused-ring divalent aromatic hydrocarbon group with 6 to 25 carbon atoms or
an optionally substituted monocyclic, linked or fused-ring divalent heteroaromatic group with 3 to 25 carbon atoms;
a, b and c each independently denote an integer in the range of 1 to 3;
d and e each independently denote an integer of 1 or 2; and
f denotes an integer of 0 or 1.
Ar¹⁰ to Ar²² preferably each independently denote
(i) a phenyl group, a biphenylyl group, a terphenylyl group, a naphthyl group, a fluorenyl group, a spirobifluorenyl group, a benzofluorenyl group, a phenanthryl group, a fluoranthenyl group, a triphenylenyl group, an anthryl group, a pyrenyl group, a pyridyl group, a carbazolyl group, a benzofuranyl group, a benzothienyl group, a dibenzofuranyl group or a dibenzothienyl group or
(ii) the group represented by (i) substituted with one or more groups selected from the group consisting of a methyl group, an ethyl group, a methoxy group, a cyano group, a deuterium atom, a fluorine atom, a phenyl group, a biphenylyl group, a naphthyl group, a phenanthryl group, a pyridyl group, a carbazolyl group, a dibenzothienyl group and a dibenzofuranyl group.
L¹ to L¹⁸ preferably each independently denote
(iii) a phenylene group, a biphenylylene group, a terphenylylene group, a naphthylene group, a pyridylene group or a fluorenylene group,
(iv) the group represented by (iii) substituted with one or more groups selected from the group consisting of a methyl group, an ethyl group, a methoxy group, a cyano group, a deuterium atom, a fluorine atom, a phenyl group, a biphenylyl group, a naphthyl group, a phenanthryl group, a pyridyl group, a carbazolyl group, a dibenzothienyl group and a dibenzofuranyl group or
(v) a single bond.
X preferably denotes
(vi) a phenylene group, a biphenylylene group, a terphenylylene group, a naphthylene group, a fluorenylene group, a pyrenediyl group, an anthracenediyl group, a dibenzothiophenediyl group, a dibenzofurandiyl group, pyridinediyl group, a carbazolediyl group, cyclohexanediyl group, an adamantanediyl group, a methanediyl group or silanediyl group or
(vii) the group represented by (vi) substituted with one or more groups selected from the group consisting of a methyl group, an ethyl group, a methoxy group, a cyano group, a deuterium atom, a fluorine atom, a phenyl group, a biphenylyl group, a naphthyl group, a phenanthryl group, a pyridyl group, a carbazolyl group, a dibenzothienyl group and a dibenzofuranyl group.

The hole-transporting triarylamine compound is, for example, a compound represented by one of the formulae (K1) to (K76):

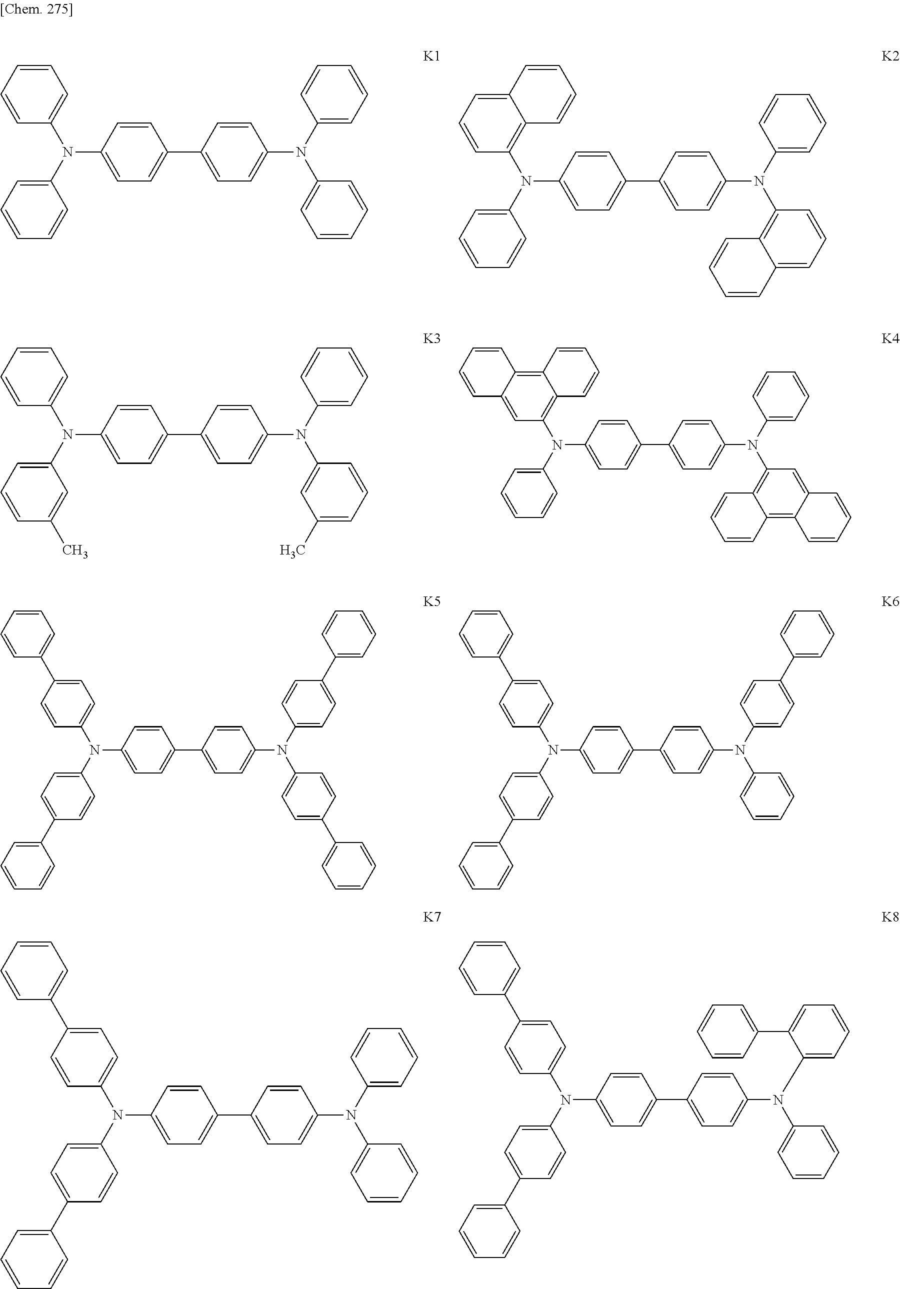

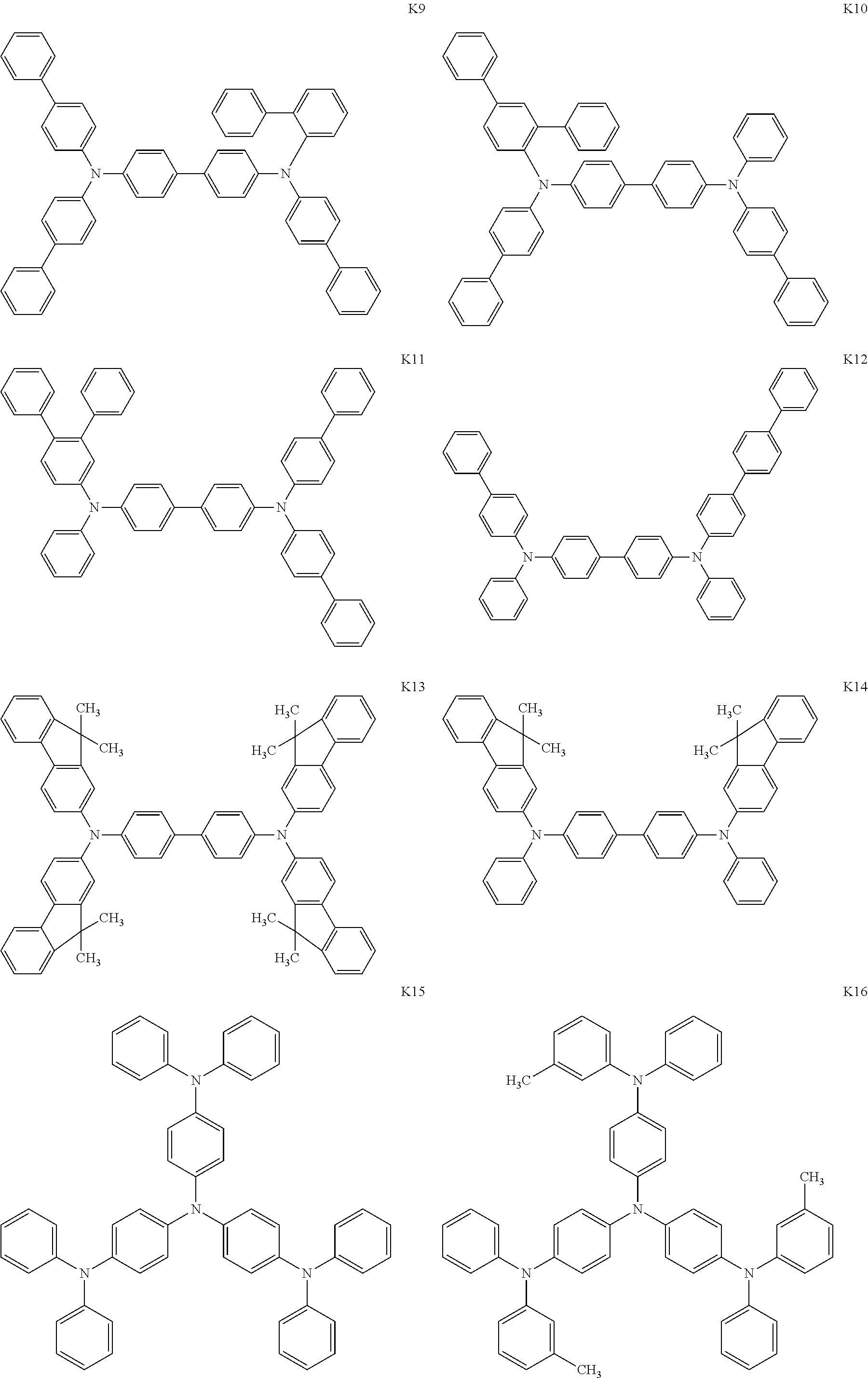

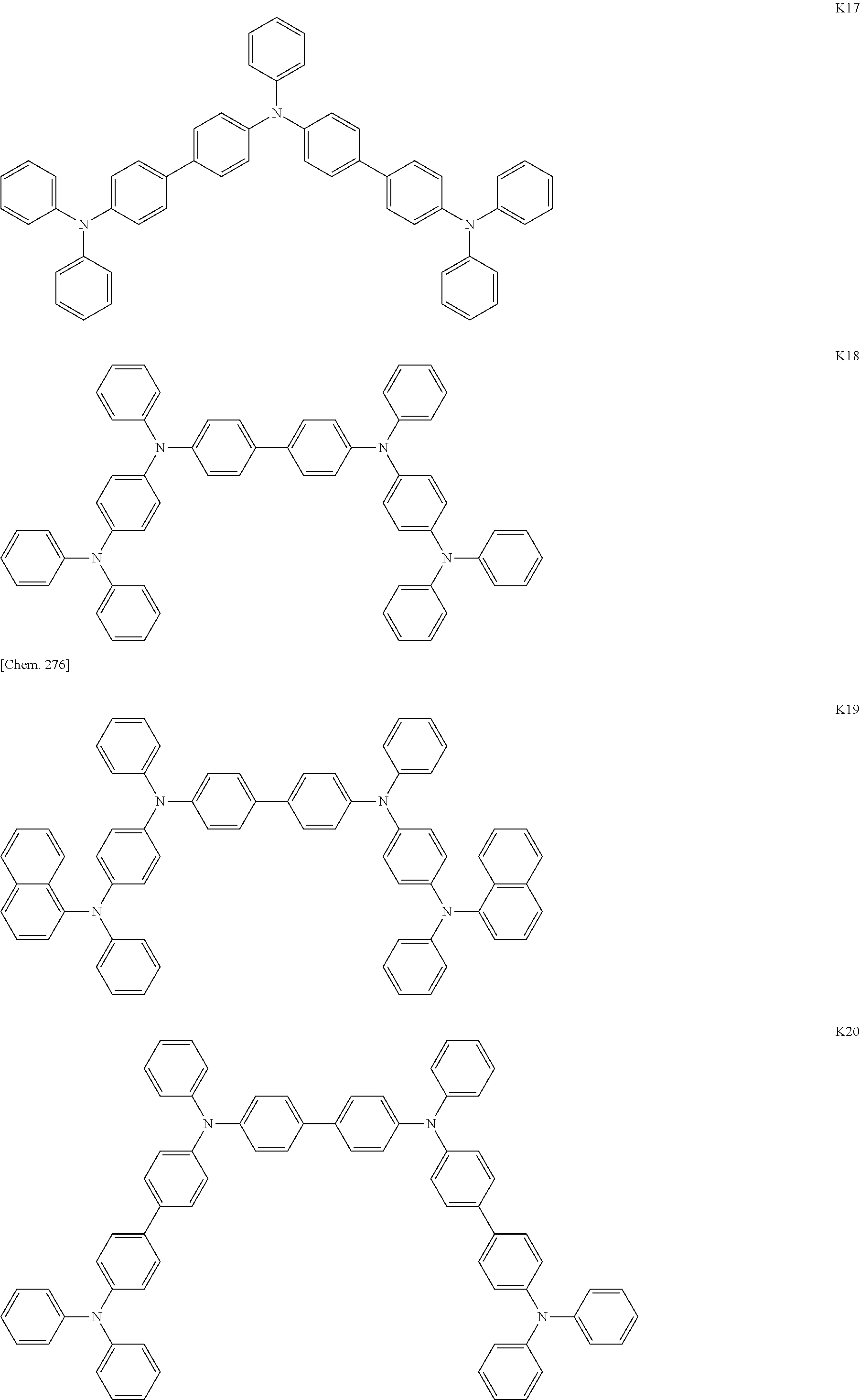 [SMILES: c1ccc(N(c2ccccc2)c2ccc(-c3ccc(N(c4ccccc4)c4ccc(-c5ccc(N(c6ccccc6)c6ccccc6)cc5)cc4)cc3)cc2)cc1 ; c1ccc(N(c2ccccc2)c2ccc(N(c3ccccc3)c3ccc(-c4ccc(N(c5ccccc5)c5ccc(N(c6ccccc6)c6ccccc6)cc5)cc4)cc3)cc2)cc1 ; c1ccc(N(c2ccc(-c3ccc(N(c4ccccc4)c4ccc(N(c5ccccc5)c5cccc6ccccc56)cc4)cc3)cc2)c2ccc(N(c3ccccc3)c3cccc4ccccc34)cc2)cc1 ; c1ccc(N(c2ccccc2)c2ccc(-c3ccc(N(c4ccccc4)c4ccc(-c5ccc(N(c6ccccc6)c6ccc(-c7ccc(N(c8ccccc8)c8ccccc8)cc7)cc6)cc5)cc4)cc3)cc2)cc1]

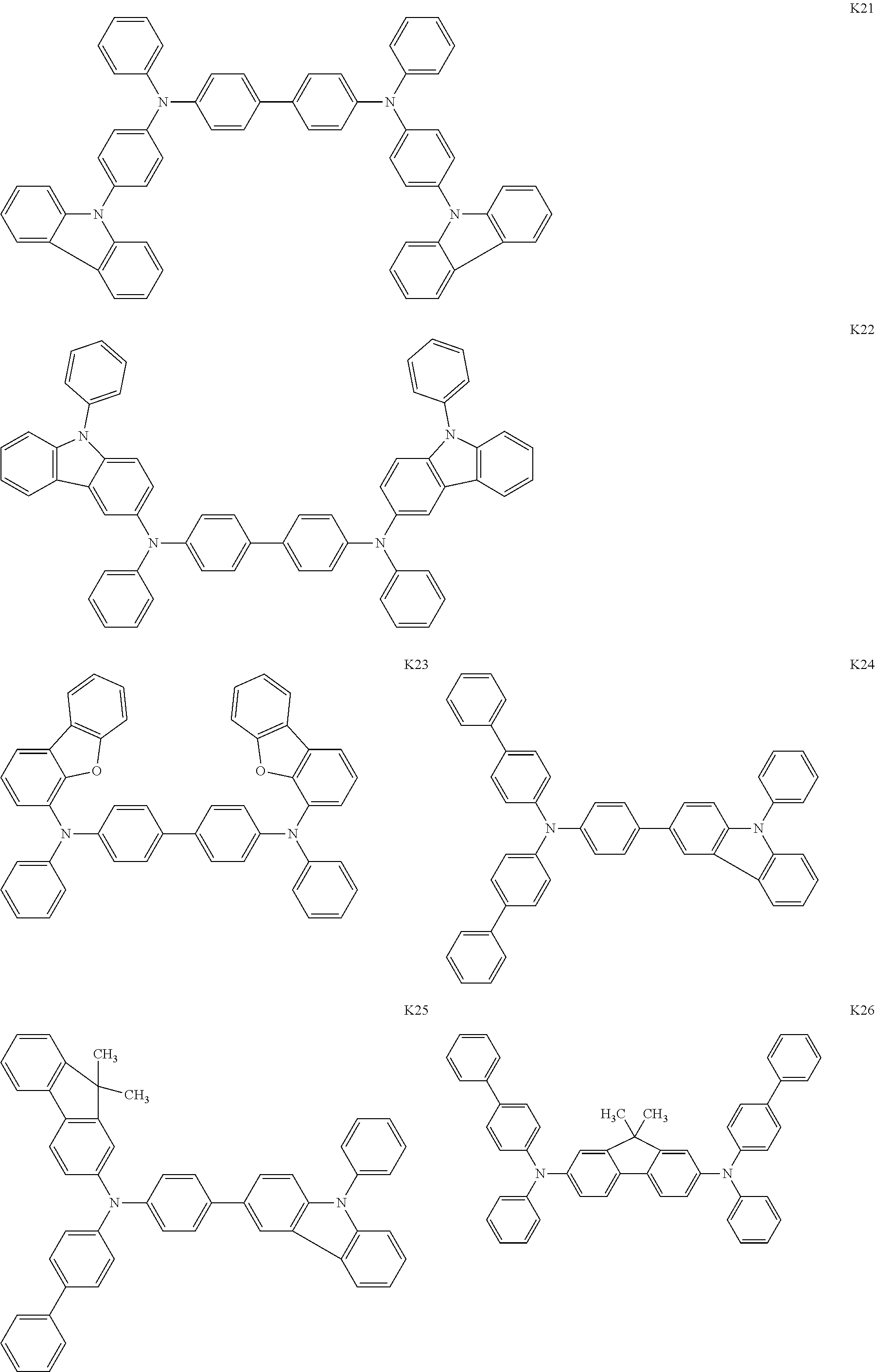

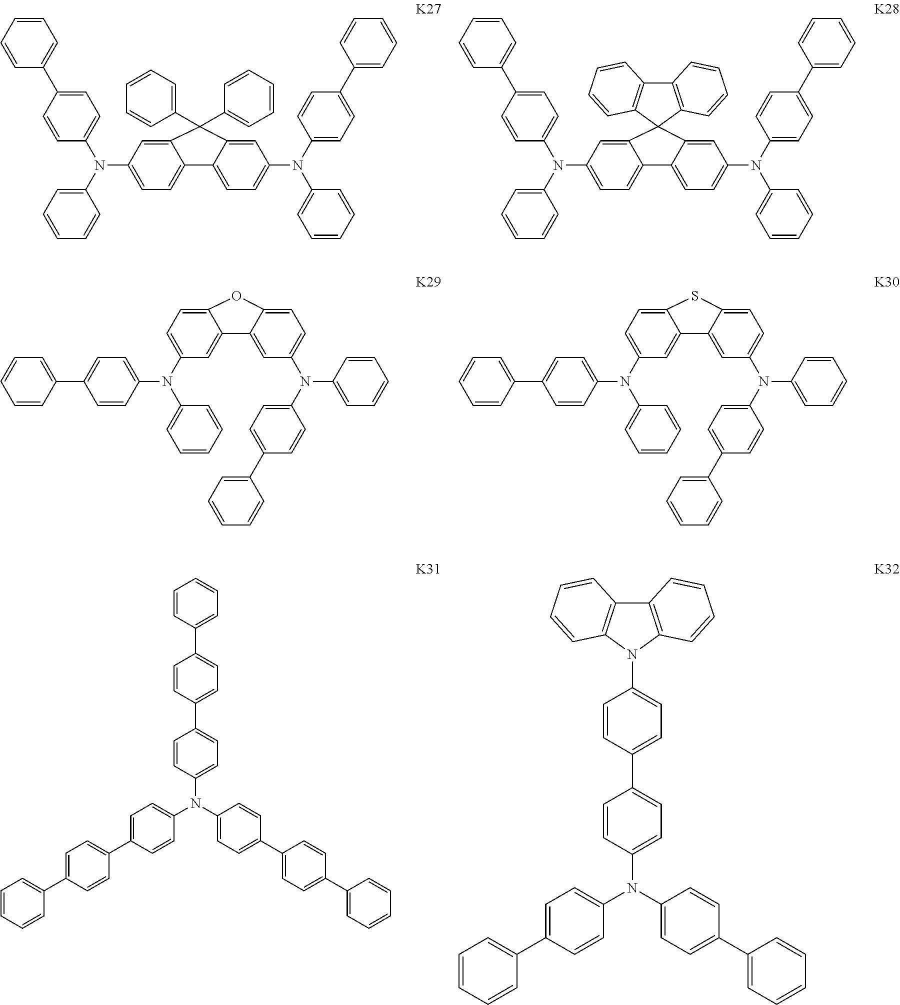

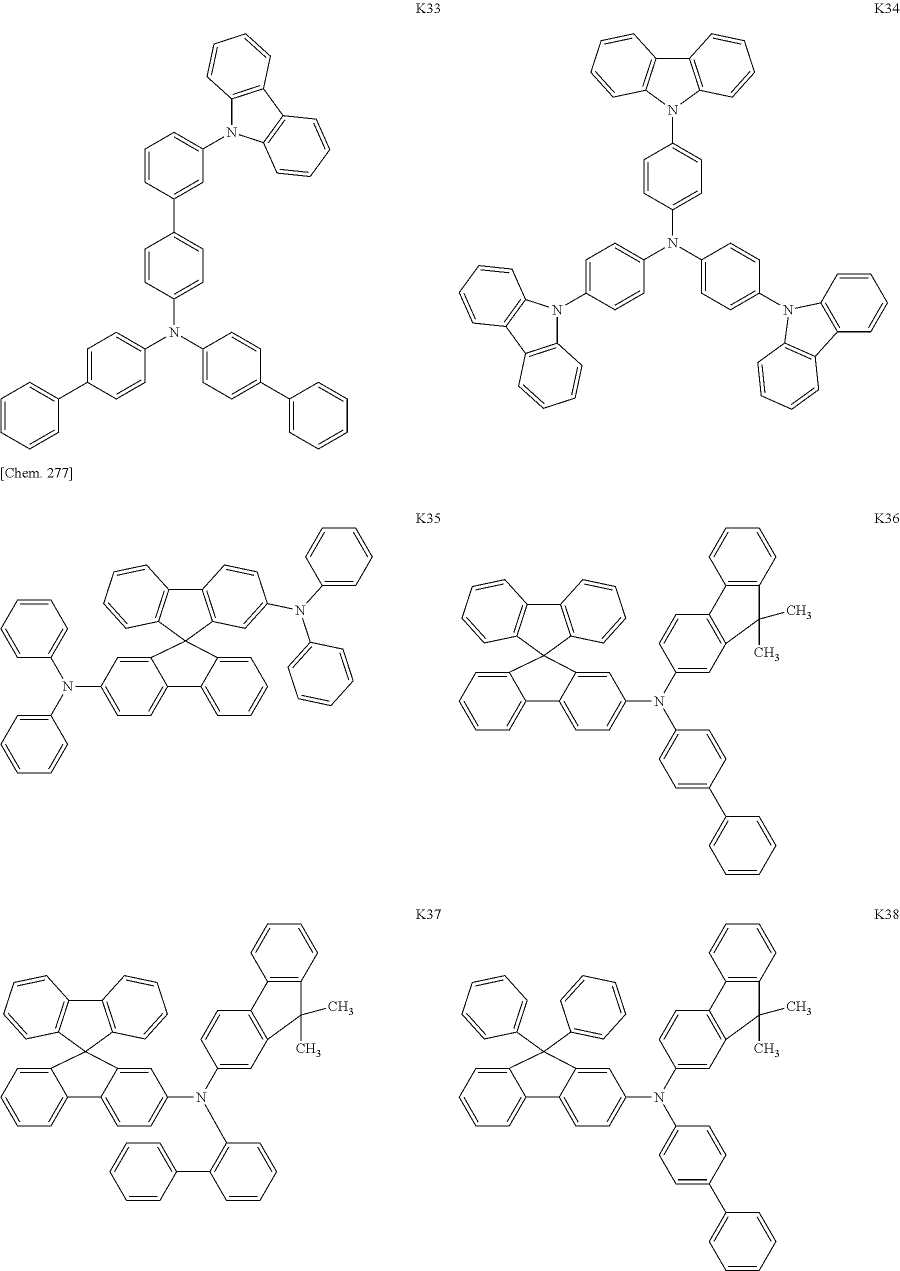

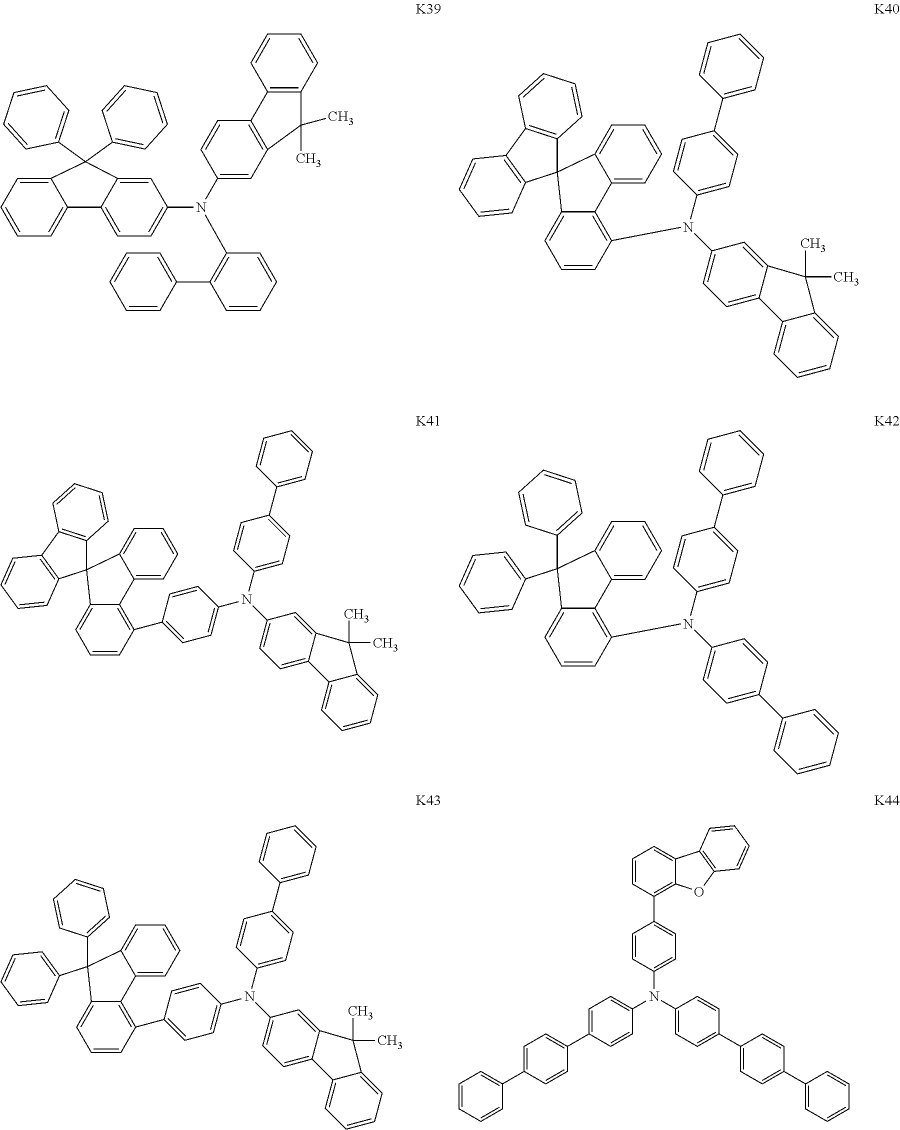

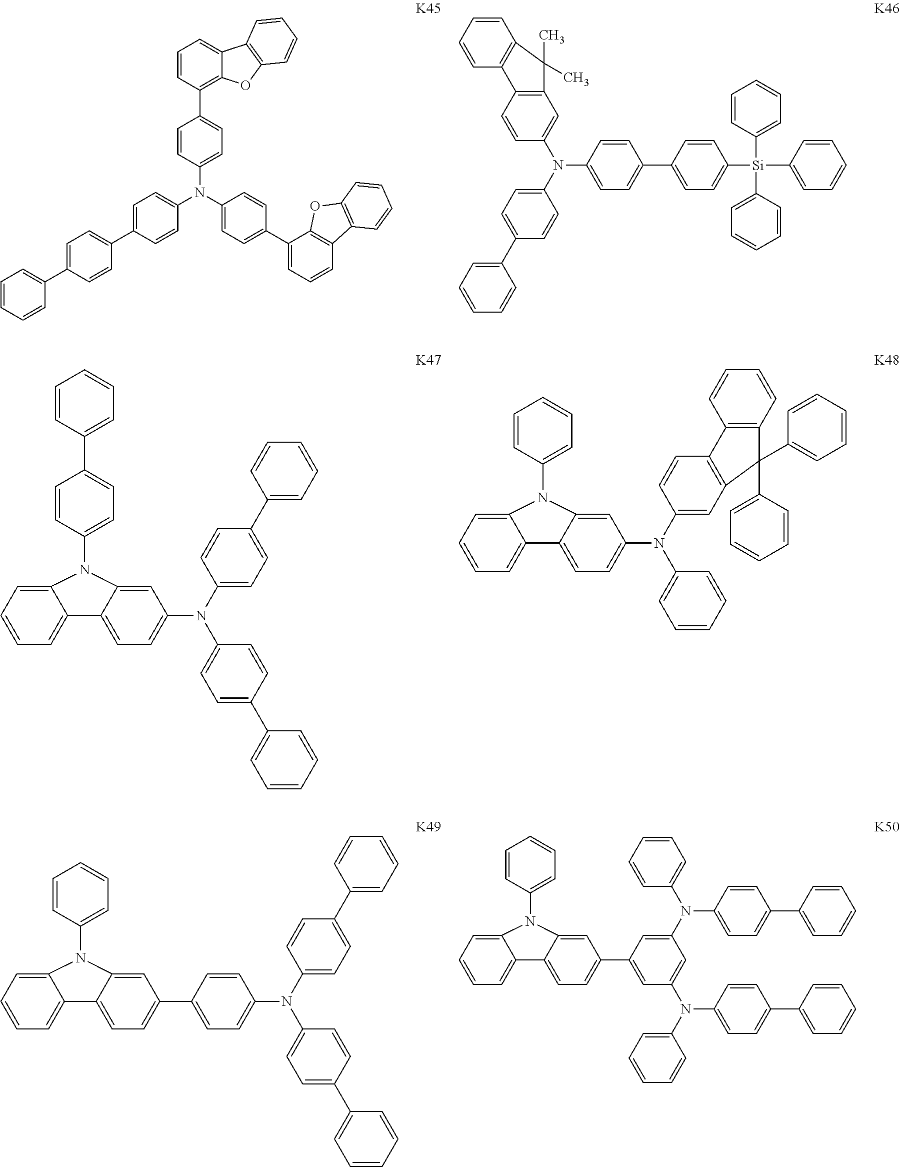

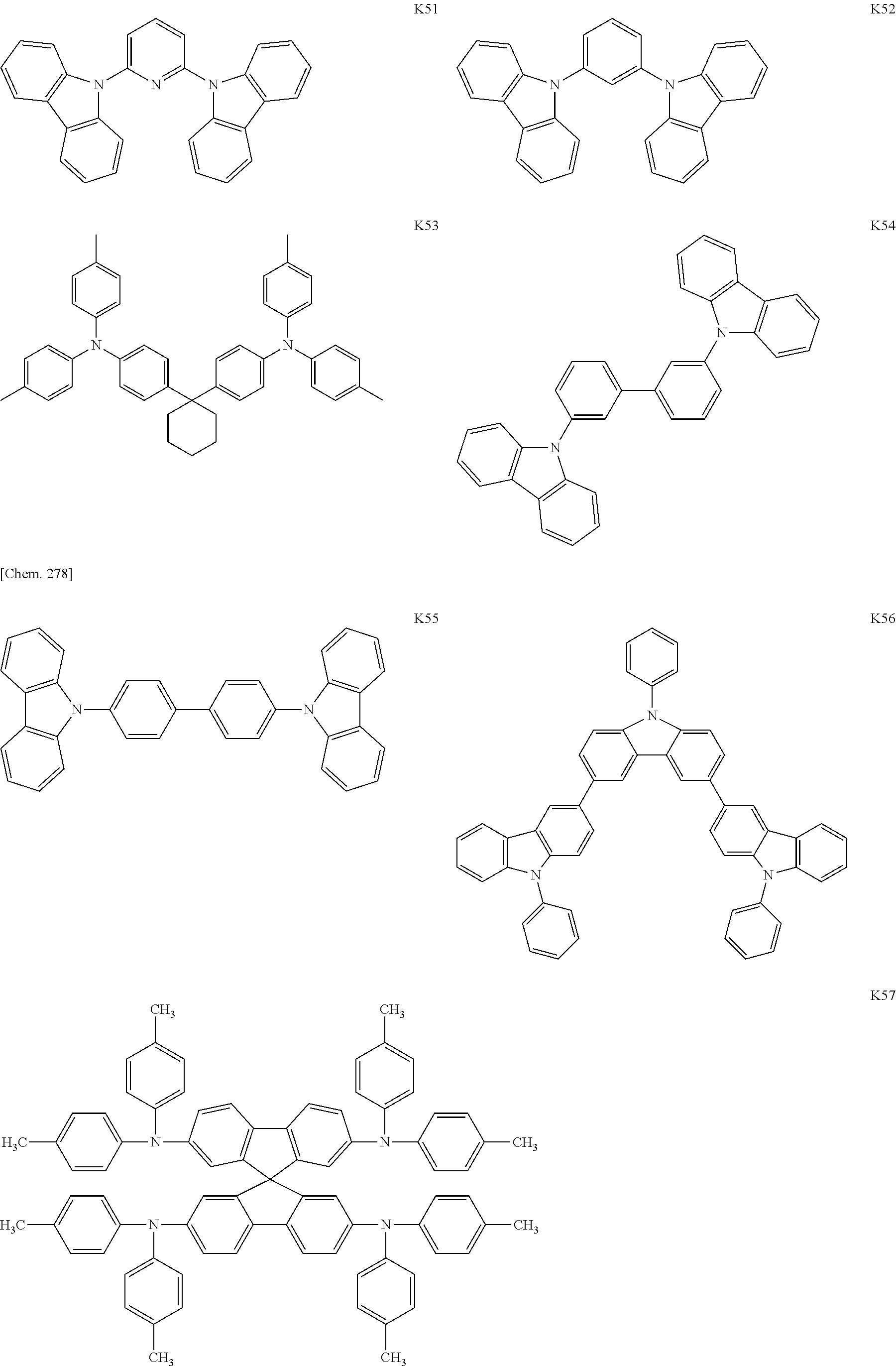

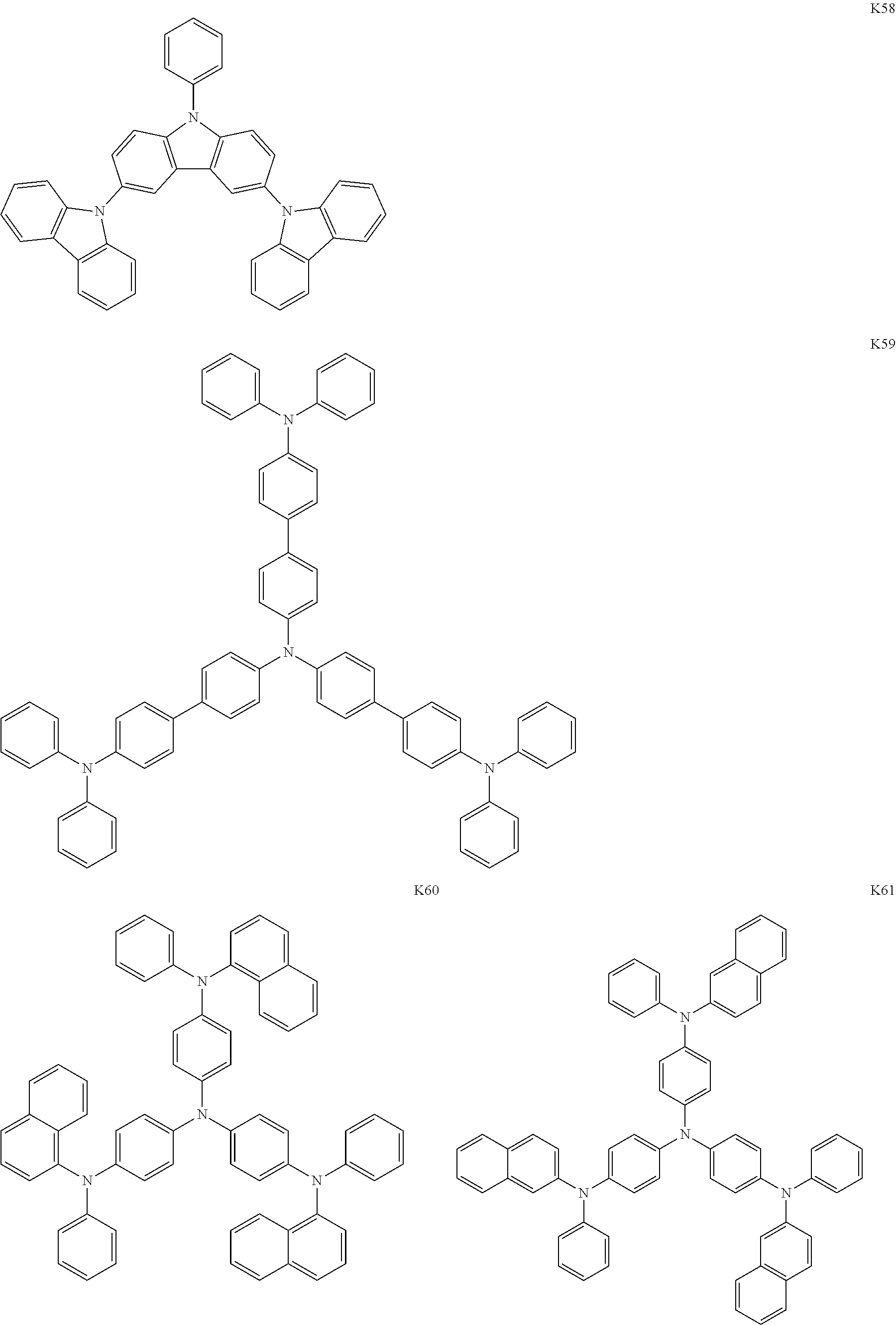 [SMILES: c1ccc(-n2c3ccc(-n4c5ccccc5c5ccccc54)cc3c3cc(-n4c5ccccc5c5ccccc54)ccc32)cc1 ; c1ccc(N(c2ccccc2)c2ccc(-c3ccc(N(c4ccc(-c5ccc(N(c6ccccc6)c6ccccc6)cc5)cc4)c4ccc(-c5ccc(N(c6ccccc6)c6ccccc6)cc5)cc4)cc3)cc2)cc1 ; c1ccc(N(c2ccc(N(c3ccc(N(c4ccccc4)c4cccc5ccccc45)cc3)c3ccc(N(c4ccccc4)c4cccc5ccccc45)cc3)cc2)c2cccc3ccccc23)cc1 ; c1ccc(N(c2ccc(N(c3ccc(N(c4ccccc4)c4ccc5ccccc5c4)cc3)c3ccc(N(c4ccccc4)c4ccc5ccccc5c4)cc3)cc2)c2ccc3ccccc3c2)cc1]

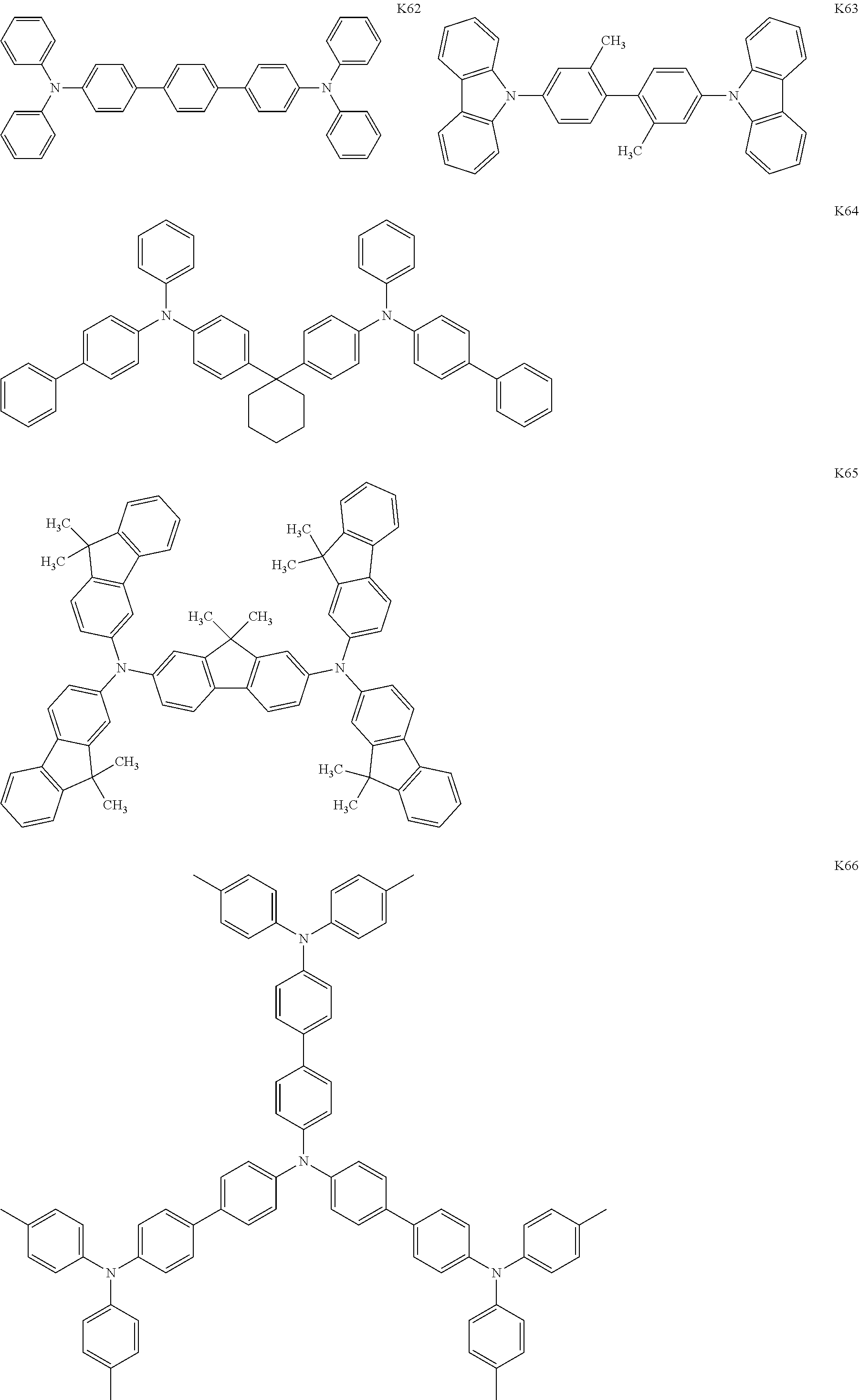

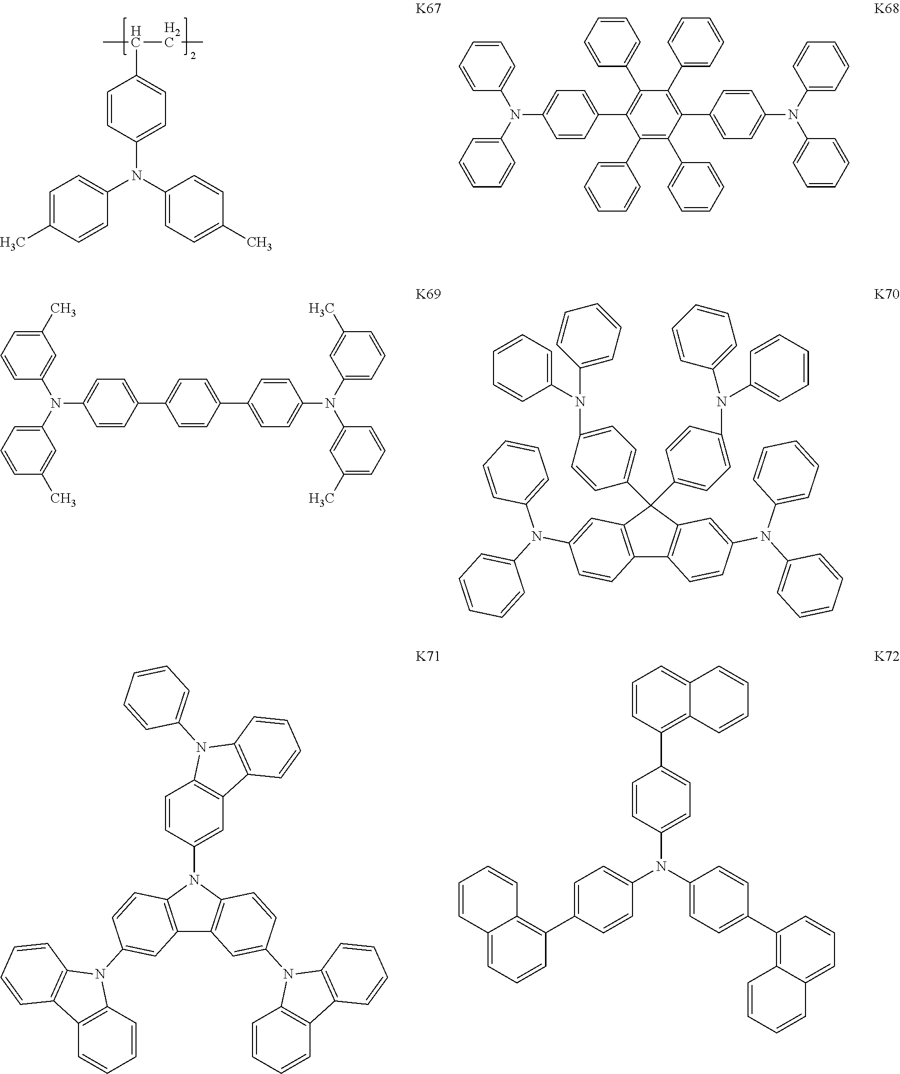

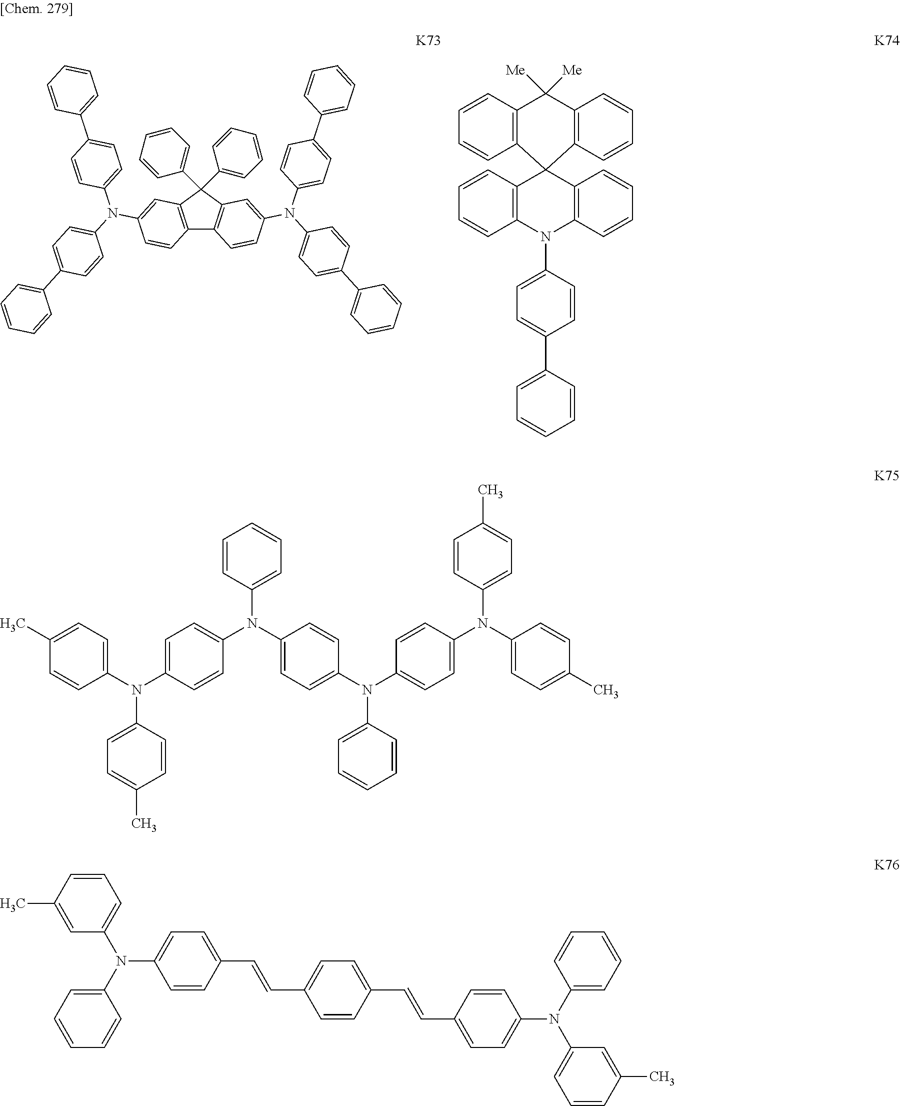

Preferably, a hole-injection layer according to an embodiment of the present disclosure contains two types of compounds, a first compound is a transverse current suppressing material represented by the formula (1) or a carbazole compound represented by the formula (22) or (23), and a second compound is an electron-accepting p-dopant.

Preferably, a hole-injection layer according to an embodiment of the present disclosure contains three types of compounds, a first compound is a transverse current suppressing material represented by the formula (1) or a carbazole compound represented by the formula (22) or (23), a second compound is an electron-accepting p-dopant, and a third compound is a hole-transporting triarylamine compound.

In the hole-injection layer, a transverse current suppressing material represented by the formula (1) or a carbazole compound represented by the formula (22) or (23) constitutes 20% or more and 99.5% or less.

### <Hole-Transport Layer 4>

The hole-transport layer 4 is provided between the hole-injection layer 3 and the electron-blocking layer 5 described later.

A hole-transport layer means a layer that is formed on a hole-injection layer to improve the mobility of a hole and to serve to improve the power efficiency of an organic light-emitting element.

A material that can transfer a hole smoothly injected from a positive electrode to a light-emitting layer and has high mobility for the hole is suitable as a hole-transport material. The hole-transport material may be any material that can be used for an organic light-emitting element and is, for example, a compound represented by one of the formulae (K1) to (K76) exemplified for the hole-injection layer.

The hole-transport layer may contain
a transverse current suppressing material represented by the formula (1) or
a carbazole compound represented by the formula (22) or (23).

Both the hole-transport layer and the hole-injection layer preferably contain
a transverse current suppressing material represented by the formula (1) or
a carbazole compound represented by the formula (22) or (23).

The hole-transport layer may have a single structure composed of one or two or more materials or may have a layered structure composed of a plurality of layers with the same composition or different compositions.

### <Electron-Blocking Layer 5>

The electron-blocking layer 5 is provided between the hole-transport layer 4 and the light-emitting layer 6 described later.

The electron-blocking layer functions as a layer for confining an electron in a light-emitting layer. More specifically, an electron injected from the negative electrode and transported from the electron-injection layer and/or the electron-transport layer to the light-emitting layer is prevented from leaking into the hole-injection layer and/or the hole-transport layer by the energy barrier present at the interface between the light-emitting layer and the electron-blocking layer. This accumulates electrons at the interface in the light-emitting layer, produces the effect of improving the luminous efficiency and provides an organic electroluminescent element with high luminescent performance.

The electron-blocking layer also has the function of transferring a hole injected from the positive electrode to the light-emitting layer and, being between the hole-transport layer and the light-emitting layer, allows more holes to be injected into the light-emitting layer at a lower electric field.

A material of the electron-blocking layer has at least one of hole-injection properties, hole-transport properties and electron barrier properties. A material of the electron-blocking layer may be an organic material or an inorganic material.

Specific examples of a material of the electron-blocking layer include triazole derivatives, oxadiazole derivatives, imidazole derivatives, polyarylalkane derivatives, pyrazoline derivatives, pyrazolone derivatives, phenylenediamine derivatives, arylamine derivatives, amino-substituted chalcone derivatives, oxazole derivatives, styrylanthracene derivatives, fluorenone derivatives, hydrazone derivatives, stilbene derivatives, silazane derivatives, aniline copolymers, electrically conductive oligomers (particularly thiophene oligomers), porphyrin compounds, aromatic tertiary amine compounds, styrylamine compounds and the like. Among these, porphyrin compounds, aromatic tertiary amine compounds and styrylamine compounds, particularly aromatic tertiary amine compounds, are preferred from the perspective of an organic electroluminescent element with good performance.

Specific examples of the aromatic tertiary amine compounds and styrylamine compounds include, but are not limited to, N,N,N′,N′-tetraphenyl-4,4′-diaminophenyl, N,N′-diphenyl-N,N′-bis(m-tolyl)-[1,1′-biphenyl]-4,4′-diamine (TPD), 2,2-bis(4-di-p-tolylaminophenyl)propane, 1,1-bis(4-di-p-tolylaminophenyl)cyclohexane, N,N,N′,N′-tetra-p-tolyl-4,4′-diaminobiphenyl, 1,1-bis(4-di-p-tolylaminophenyl)-4-phenylcyclohexane, bis(4-dimethylamino-2-methylphenyl)phenylmethane, bis(4-di-p-tolylaminophenyl)phenylmethane, N,N′-diphenyl-N,N′-di(4-methoxyphenyl)-4,4′-diaminobiphenyl, N,N,N′,N′-tetraphenyl-4,4′-diaminodiphenyl ether, 4,4′-bis(diphenylamino)quadriphenyl, N,N,N-tri(p-tolyl)amine, 4-(di-p-tolylamino)-4′-[4-(di-p-tolylamino)styryl]stilbene, 4-N,N-diphenylamino-(2-diphenylvinyl)benzene, 3-methoxy-4′-N,N-diphenylaminostilbenzene, N-phenylcarbazole, 4,4′-bis[N-(1-naphthyl)-N-phenylamino]biphenyl (NPD), 4,4′,4″-tris[N-(m-tolyl)-N-phenylamino]triphenylamine (MTDATA) and the like.

The electron-blocking layer may have a single structure composed of one or two or more materials or may have a layered structure composed of a plurality of layers with the same composition or different compositions.

A transverse current suppressing material represented by the formula (1) or a carbazole compound represented by the formula (22) or (23) may be used for the electron-blocking layer.

### <Light-Emitting Layer 6>

The light-emitting layer 6 is provided between the electron-blocking layer 5 and the electron-transport layer 7 described later.

A material of the light-emitting layer may be a phosphorescent material, a fluorescent material or a thermally activated delayed fluorescent material. In the light-emitting layer, a pair of an electron and a hole recombines and emits light.

The light-emitting layer may be composed of a single low-molecular-weight material or a single polymer material and is more commonly composed of a host material doped with a guest compound. Light emission arises primarily from the dopant and can have any color.

The host material is, for example, a compound with a biphenylyl group, a fluorenyl group, a triphenylsilyl group, a carbazole group, a pyrenyl group or an anthryl group. Specific examples include, but are not limited to, DPVBi (4,4′-bis(2,2-diphenylvinyl)-1,1′-biphenyl), BCzVBi (4,4′-bis(9-ethyl-3-carbazovinylene)1,1′-biphenyl), TBADN (2-tertiary-butyl-9,10-di(2-naphthyl)anthracene), ADN (9,10-di(2-naphthyl)anthracene), CBP (4,4′-bis(carbazol-9-yl)biphenyl), CDBP (4,4′-bis(carbazol-9-yl)-2,2′-dimethylbiphenyl), 2-(9-phenylcarbazol-3-yl)-9-[4-(4-phenylphenylquinazolin-2-yl)carbazole and 9,10-bis(biphenyl)anthracene.

Examples of fluorescent dopants include, but are not limited to, anthracene, pyrene, tetracene, xanthene, perylene, rubrene, coumarin, rhodamine, quinacridone, dicyanomethylenepyran compounds, thiopyran compounds, polymethine compounds, pyrylium, thiapyrylium compounds, fluorene derivatives, periflanthene derivatives, indenoperylene derivatives, bis(azinyl)amine boron compounds, bis(azinyl)methane compounds, carbostyril compounds, boron compounds and cyclic amine compounds. A combination of two or more selected from these may be used as a fluorescent dopant.

Examples of phosphorescent dopants include, but are not limited to, organometallic complexes of transition metals, such as iridium, platinum, palladium and osmium.

Specific examples of the fluorescent dopants and phosphorescent dopants include, but are not limited to, Alq3 (tris(8-hydroxyquinoline)aluminum), DPAVBi (4,4′-bis[4-(di-p-tolylamino)styryl]biphenyl), perylene, bis[2-(4-n-hexylphenyl)quinoline](acetylacetonate)iridium (III), Ir(PPy)3 (tris(2-phenylpyridine)iridium (III)), FlrPic (bis(3,5-difluoro-2-(2-pyridyl)phenyl-(2-carboxypyridyl)iridium (III))) and the like.

The light-emitting materials are not limited to being contained only in the light-emitting layer. For example, the light-emitting materials may be contained in a layer (the electron-blocking layer 5 or the electron-transport layer 7) adjacent to the light-emitting layer. This can further increase the luminous efficiency of the organic electroluminescent element.

The light-emitting layer may have a monolayer structure composed of one or two or more materials or may have a layered structure composed of a plurality of layers with the same composition or different compositions.

### <Electron-Transport Layer 7>

The electron-transport layer 7 is provided between the light-emitting layer 6 and the electron-injection layer 8 described later.

The electron-transport layer has the function of transferring an electron injected from the negative electrode to the light-emitting layer. The electron-transport layer between the negative electrode and the light-emitting layer allows an electron to be injected into the light-emitting layer at a lower electric field.

Specific examples of a material of the electron-transport layer include tris(8-quinolinolato)aluminum derivatives, imidazole derivatives, benzimidazole derivatives, triazine derivatives, pyrimidine derivatives, pyridine derivatives, pyrazine derivatives, quinoline derivatives, quinoxaline derivatives, oxadiazole derivatives, phosphole derivatives, silole derivatives, phosphine oxide derivatives and the like. Among these, triazine derivatives and pyrimidine derivatives are preferred from the perspective of an organic electroluminescent element with good performance.

The electron-transport layer may further contain one or more selected from known electron-transport materials in addition to the materials described above.

The known electron-transport materials may be alkali metal complexes, alkaline-earth metal complexes, earth metal complexes and the like. Examples of the alkali metal complexes, alkaline-earth metal complexes and earth metal complexes include 8-hydroxyquinolinate lithium (Liq), bis(8-hydroxyquinolinate)zinc, bis(8-hydroxyquinolinate)copper, bis(8-hydroxyquinolinate)manganese, tris(8-hydroxyquinolinate)aluminum, tris(2-methyl-8-hydroxyquinolinate)aluminum, tris(8-hydroxyquinolinate)gallium, bis(10-hydroxybenzo[h]quinolinato)beryllium, bis(10-hydroxybenzo[h]quinolinato)zinc, bis(2-methyl-8-quinolinato)chlorogallium, bis(2-methyl-8-quinolinato)(o-cresolate)gallium, bis(2-methyl-8-quinolinato)-1-naphtholate aluminum, bis(2-methyl-8-quinolinato)-2-naphtholate gallium and the like. Inorganic compounds, such as Yb, Li, and Ca, may also be used.

The electron-transport layer may have a monolayer structure composed of one or two or more materials or may have a layered structure composed of a plurality of layers with the same composition or different compositions.

### <Electron-Injection Layer 8>

The electron-injection layer 8 is provided between the electron-transport layer 7 and the negative electrode 9 described later.

The electron-injection layer has the function of transferring an electron injected from the negative electrode to the light-emitting layer. The electron-injection layer between the negative electrode and the light-emitting layer allows an electron to be injected into the light-emitting layer at a lower electric field.

Examples of a material of the electron-injection layer include organic compounds, such as fluorenone, anthraquinodimethane, diphenoquinone, thiopyran dioxide, oxazole, oxadiazole, triazole, imidazole, perylenetetracarboxylic acid, fluorenylidenemethane, anthraquinodimethane and anthrone. Examples of a material of the electron-injection layer also include various oxides, such as SiO2, AlO, SiN, SiON, AlON, GeO, LiO, LiON, TiO, TiON, TaO, TaON, TaN, LiF, C and Yb and inorganic compounds, such as fluorides, nitrides and oxynitrides.

### <Negative Electrode 9>

The negative electrode 9 is provided on the electron-injection layer 8.

In an organic electroluminescent element in which only light emission through the positive electrode is extracted, the negative electrode can be formed of any electrically conductive material.

A material of the negative electrode is, for example, a metal with a low work function (hereinafter also referred to as an electron-injecting metal), an alloy, an electrically conductive compound or a mixture thereof. The metal with a low work function is, for example, a metal with a work function of 4 eV or less.

Specific examples of a material of the negative electrode include sodium, sodium-potassium alloys, magnesium, lithium, magnesium/copper mixtures, magnesium/silver mixtures, magnesium/aluminum mixtures, magnesium/indium mixtures, aluminum/aluminum oxide (Al₂O₃) mixtures, indium, lithium/aluminum mixtures, rare-earth metals and the like.

Among these, a mixture of an electron-injecting metal and a second metal, which is a stable metal with a higher work function than the electron-injecting metal, for example, a magnesium/silver mixture, a magnesium/aluminum mixture, a magnesium/indium mixture, an aluminum/aluminum oxide (Al₂O₃) mixture, a lithium/aluminum mixture or the like is preferred in terms of the electron-injection properties and the durability against oxidation or the like.

Next, a method for producing the transverse current suppressing material (1) is described below.

The transverse current suppressing material (1) can be produced by a method including the synthetic routes (p) to (s) or by another method.

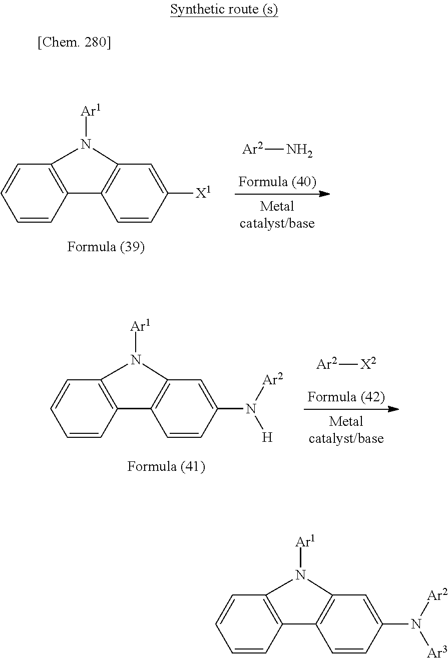

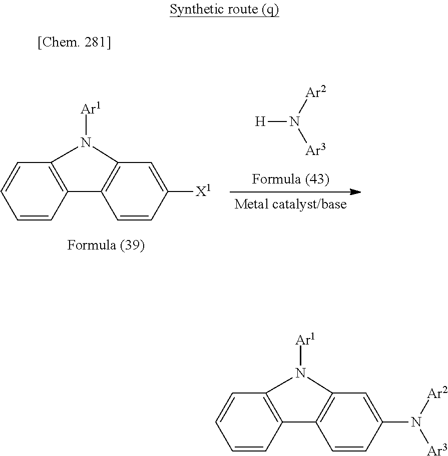

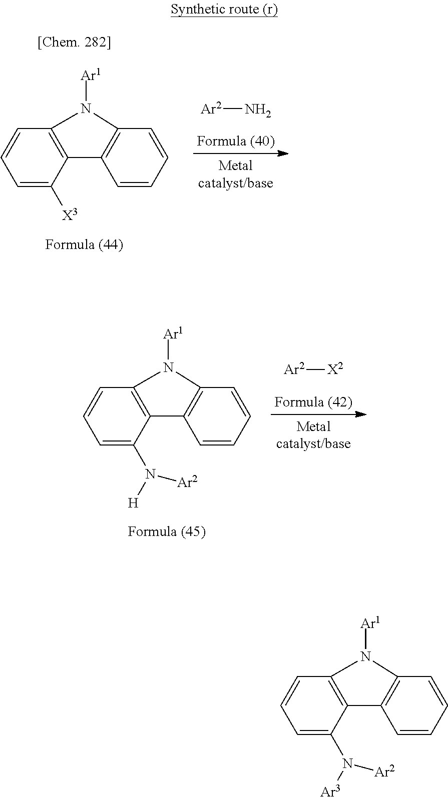

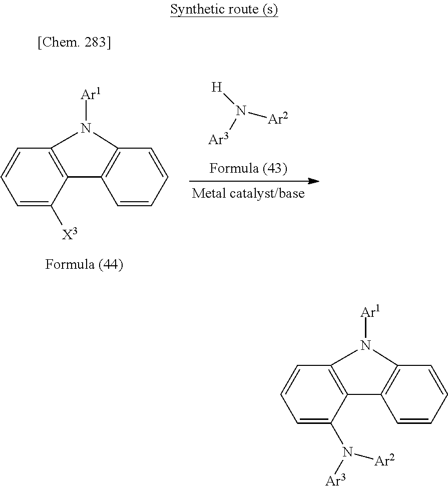

In the formulae (39) to (45),
the definitions of Ar¹, Ar² and Ar³ are the same as the definitions of Ar¹, Ar² and Ar³ in the formula (1), respectively;
X¹, X² and X³ each independently denote a halogen atom; and
the halogen atom represented by X¹, X² and X³ is, for example, a fluorine atom, a chlorine atom, a bromine atom or an iodine atom, and a chlorine atom or a bromine atom is preferred in terms of a high yield of the transverse current suppressing material (1).

The reactions in the synthetic routes (p) to (s) are methods in which a halide represented by the formula (39), (42) or (44) and an amine compound represented by the formula (40), (41), (43) or (45) are reacted in the presence of a palladium catalyst and a base, and reaction conditions of a typical Buchwald-Hartwig amination reaction can be applied.

The halogenated carbazole compounds (39) and (44) can be produced, for example, in accordance with Japanese Patent No. 5609256 and No. 6115075, respectively. Commercial products may also be used.

A palladium catalyst used in the amination reaction is, for example, a palladium salt, such as palladium chloride, palladium acetate, palladium trifluoroacetate or palladium nitrate. Other examples include complex compounds, such as a π-allyl palladium chloride dimer, palladium acetylacetonate, tris(dibenzylideneacetone)dipalladium, bis(dibenzylideneacetone)palladium, dichlorobis(acetonitrile)palladium and dichlorobis(benzonitrile)palladium; and palladium complexes with a tertiary phosphine as a ligand, such as dichlorobis(triphenylphosphine)palladium, tetrakis(triphenylphosphine)palladium, dichloro(1,1′-bis(diphenylphosphino)ferrocene)palladium, bis(tri-tert-butylphosphine)palladium, bis(tricyclohexylphosphine)palladium and dichlorobis(tricyclohexylphosphine)palladium. These can also be prepared in a reaction system by adding a tertiary phosphine to a palladium salt or a complex compound.

The tertiary phosphine is, for example, triphenylphosphine, trimethylphosphine, tributylphosphine, tri(tert-butyl)phosphine, tricyclohexylphosphine, tert-butyldiphenylphosphine, 9,9-dimethyl-4,5-bis(diphenylphosphino)xanthene, 2-(diphenylphosphino)-2′-(N,N-dimethylamino)biphenyl, 2-(di-tert-butylphosphino)biphenyl, 2-(dicyclohexylphosphino)biphenyl, bis(diphenylphosphino)methane, 1,2-bis(diphenylphosphino)ethane, 1,3-bis(diphenylphosphino)propane, 1,4-bis(diphenylphosphino)butane, 1,1′-bis(diphenylphosphino)ferrocene, tri(2-furyl)phosphine, tri(o-tolyl)phosphine, tris(2,5-xylyl)phosphine, (±)-2,2′-bis(diphenylphosphino)-1,1′-binaphthyl or 2-dicyclohexylphosphino-2′,4′,6′-triisopropylbiphenyl.

Among these, a palladium complex with a tertiary phosphine as a ligand is preferred in terms of a high yield, and a palladium complex with 2-dicyclohexylphosphino-2′,4′,6′-triisopropylbiphenyl, tri(o-tolyl)phosphine, tri(tert-butyl)phosphine, 9,9-dimethyl-4,5-bis(diphenylphosphino)xanthene or tricyclohexylphosphine as a ligand is more preferred.

The mole ratio of the tertiary phosphine to the palladium salt or complex compound preferably ranges from 1:10 to 10:1, more preferably 1:2 to 3:1 in terms of a high yield. The amount of the palladium catalyst used in the amination reaction is preferably, but not limited to, in the range of 0.005 to 0.5 molar equivalent with respect to the amine compound in terms of a high yield.

A base used in the amination reaction is, for example, a metal hydroxide, such as sodium hydroxide, potassium hydroxide or calcium hydroxide; a metal carbonate, such as sodium carbonate, potassium carbonate, lithium carbonate or cesium carbonate; a metal acetate, such as potassium acetate or sodium acetate; a metal phosphate, such as potassium phosphate or sodium phosphate; a metal fluoride, such as sodium fluoride, potassium fluoride or cesium fluoride; a metal alkoxide, such as sodium methoxide, potassium methoxide, sodium ethoxide, potassium isopropyl oxide, potassium tert-butoxide or potassium tert-butoxide; or the like. Among these, potassium tert-butoxide is preferred in terms of a high reaction yield. The base may be used in any amount. In terms of a high reaction yield, the mole ratio of the base to the amine compound preferably ranges from 1:2 to 10:1, more preferably 1:1 to 4:1.

The coupling reaction and the boration reaction described above may be performed in a solvent.

The solvent may be water, an ether, such as diisopropyl ether, dibutyl ether, cyclopentyl methyl ether (CPME), tetrahydrofuran (THF), 2-methyltetrahydrofuran, 1,4-dioxane or dimethoxyethane; an aromatic hydrocarbon, such as benzene, toluene, xylene, mesitylene or tetralin; a carbonate, such as ethylene carbonate, propylene carbonate, dimethyl carbonate, diethyl carbonate, ethyl methyl carbonate or 4-fluoroethylene carbonate; an ester, such as ethyl acetate, butyl acetate, methyl propionate, ethyl propionate, methyl butyrate or γ-lactone; an amide, such as N,N-dimethylformamide (DMF), dimethylacetamide (DMAc) or N-methylpyrrolidone (NMP); a urea, such as N,N,N′,N′-tetramethylurea (TMU) or N,N′-dimethylpropyleneurea (DMPU); an alcohol, such as dimethyl sulfoxide (DMSO), methanol, ethanol, isopropyl alcohol, butanol, octanol, benzyl alcohol, ethylene glycol, propylene glycol, diethylene glycol, triethylene glycol or 2,2,2-trifluoroethanol; or the like. These may be used alone or in a mixture at any ratio. The solvent may be used in any amount. Among these, in terms of a high reaction yield, an aromatic hydrocarbon is preferred, and toluene or xylene is more preferred.

The coupling reaction and the boration reaction can be performed at a temperature appropriately selected from 0° C. to 200° C., preferably 60° C. to 160° C. in terms of a high reaction yield.

In the amination reaction, the target product can be produced by appropriately combining typical purification treatments, such as recrystallization, column chromatography, sublimation purification and preparative HPLC, after completion of the reaction, if necessary.

### EXAMPLES

The present invention is described in more detail below on the basis of examples. However, the present invention is not to be interpreted as being limited by these examples.

### [¹H-NMR Measurement]

¹H-NMR was measured with Bruker ASCEND HD (400 MHz, manufactured by BRUKER). ¹H-NMR was measured using deuterochloroform (CDCl₃) as a measurement solvent and tetramethylsilane (TMS) as an internal standard substance.

### [Field Desorption Mass Spectroscopy (FDMS) Measurement]

FDMS measurement was performed with M-80B manufactured by Hitachi, Ltd.

### [Measurement of Transverse Current]

The transverse current was measured with a source meter 2400 manufactured by Keithley Instruments, Inc.

### [Measurement of Organic Electroluminescent Element]

The emission properties of an organic electroluminescent element were evaluated by applying a direct current to a prepared element at room temperature and using a luminance meter (product name: BM-9, manufactured by Topcon Technohouse Corporation).

### Synthesis Example 1 (Synthesis of 2-chloro-9-(biphenyl-4-yl)carbazole)

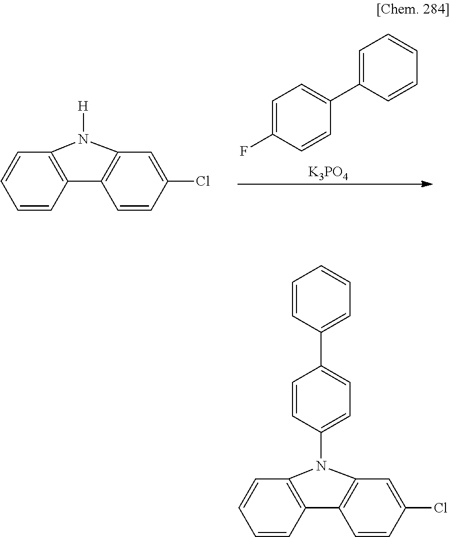

9.8 g (48 mmol) of 2-chloro-9H-carbazole, 10 g (58 mmol) of 4-fluorobiphenyl, 21 g (96 mmol) of tripotassium phosphate and 230 mL of dimethyl sulfoxide were added to a 500-mL three-neck flask and were stirred at 180° C. for 16 hours. After cooling to room temperature, 230 mL of pure water was added and stirred to precipitate a solid. The solid was then filtered off and washed with water and hexane. The solid was recrystallized with a mixed solvent of toluene and butanol to isolate 14 g (40 mmol) of 2-chloro-9-(biphenyl-4-yl)carbazole as a white solid (yield: 80%).

The compound was identified by ¹H NMR measurement.

¹H-NMR (CDCl₃); 8.11 (d, J=8.0 Hz, 1H), 8.01 (d, J=8.0 Hz, 1H), 7.81-7.85 (m, 2H), 7.68-7.72 (m, 2H), 7.60-7.63 (m, 2H), 7.51 (t, J=8.0 Hz, 2H), 7.41-7.46 (m, 4H), 7.29-7.34 (m, 1H), 7.24-7.28 (m, 1H)

### Synthesis Example 2 (Synthesis of (2-chloro-9-(biphenyl-2-yl)carbazole)

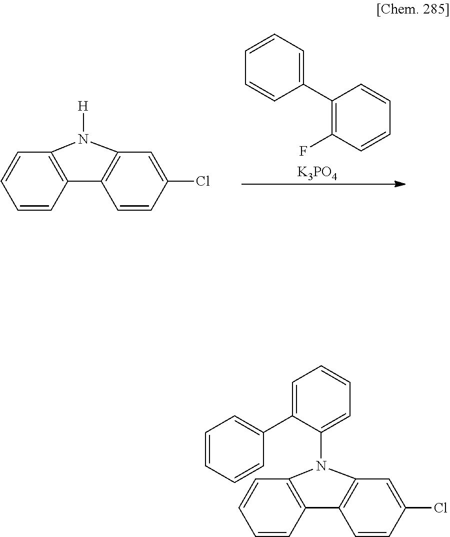

9.8 g (48 mmol) of 2-chloro-9H-carbazole, 10 g (58 mmol) of 2-fluorobiphenyl, 21 g (96 mmol) of tripotassium phosphate and 230 mL of dimethyl sulfoxide were added to a 500-mL three-neck flask and were stirred at 180° C. for 16 hours. After cooling to room temperature, 230 mL of pure water was added and stirred to precipitate a solid. The solid was then filtered off and washed with water and hexane. The solid was recrystallized with a mixed solvent of toluene and butanol to isolate 12 g (34 mmol) of 2-chloro-9-(biphenyl-2-yl)carbazole as a white solid (yield: 71%).

The compound was identified by ¹H NMR measurement.

¹H-NMR (CDCl₃); 7.98 (d, J=8.0, 1H), 7.91 (d, J=8.0, 1H), 7.54-7.68 (m, 3H), 7.46-7.50 (m, 1H), 7.24-7.30 (m, 1H), 7.16-7.22 (m, 1H), 7.10-7.15 (m, 1H), 6.97-7.08 (m, 7H)

### Synthesis Example 3 (Synthesis of (2-chloro-9-(m-terphenyl-4′-yl)carbazole)

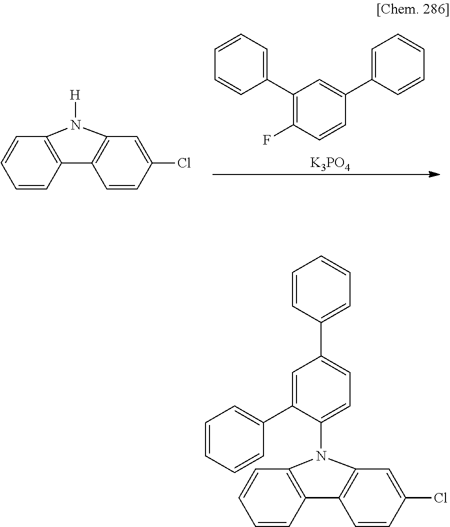

8.2 g (40 mmol) of 2-chloro-9H-carbazole, 12 g (48 mmol) of 4′-fluoro-m-terphenyl, 17 g (80 mmol) of tripotassium phosphate and 200 mL of dimethyl sulfoxide were added to a 500-mL three-neck flask and were stirred at 180° C. for 16 hours. After cooling to room temperature, 200 mL of pure water was added and stirred to precipitate a solid. The solid was then filtered off and washed with water and hexane. The solid was recrystallized with a mixed solvent of toluene and butanol to isolate 14 g (33 mmol) of 2-chloro-9-(m-terphenyl-4′-yl)carbazole as a white solid (yield: 79%).

The compound was identified by ¹H NMR measurement.

¹H-NMR (CDCl₃); 8.00 (d, J=8.0 Hz, 1H), 7.93 (d, J=8.0 Hz, 1H), 7.88 (d, J=8.0 Hz, 1H), 7.71-7.80 (m, 3H), 7.49-7.58 (m, 3H), 7.41-7.46 (m, 1H), 7.27-7.33 (m, 1H), 7.17-7.23 (m, 1H), 7.09-7.16 (m, 3H), 6.98-7.09 (m, 5H)

### Synthesis Example 4 (Synthesis of 2-chloro-9-(p-terphenyl-2′-yl)carbazole)

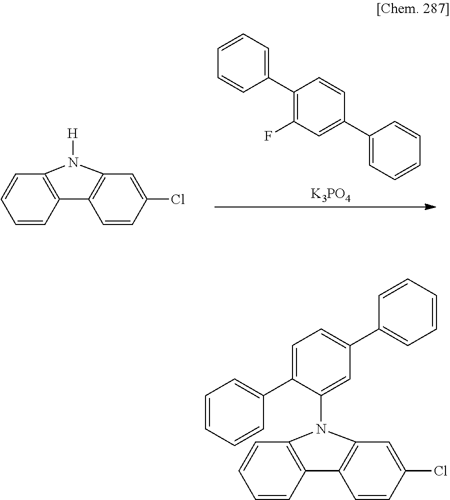

8.2 g (40 mmol) of 2-chloro-9H-carbazole, 12 g (48 mmol) of 2′-fluoro-p-terphenyl, 17 g (80 mmol) of tripotassium phosphate and 200 mL of dimethyl sulfoxide were added to a 500-mL three-neck flask and were stirred at 180° C. for 16 hours. After cooling to room temperature, 200 mL of pure water was added and stirred to precipitate a solid. The solid was then filtered off and washed with water and hexane. The solid was recrystallized with a mixed solvent of toluene and butanol to isolate 15 g (35 mmol) of 2-chloro-9-(p-terphenyl-2′-yl)carbazole as a white solid (yield: 85%).

The compound was identified by ¹H NMR measurement.

¹H-NMR (CDCl₃); 8.01 (d, J=8.0, 1H), 7.93 (d, J=8.0, 1H), 7.84-7.88 (m, 1H), 7.71-7.77 (m, 2H), 7.64-7.69 (m, 2H), 7.44-7.50 (m, 2H), 7.36-7.41 (m, 1H), 7.28-7.33 (m, 1H), 7.18-7.23 (m, 1H), 7.12-7.17 (m, 2H), 7.08-7.11 (m, 1H), 6.98-7.06 (m, 5H)

### Synthesis Example 5 (Synthesis of 2-chloro-9-(m-terphenyl-2′-yl)carbazole)

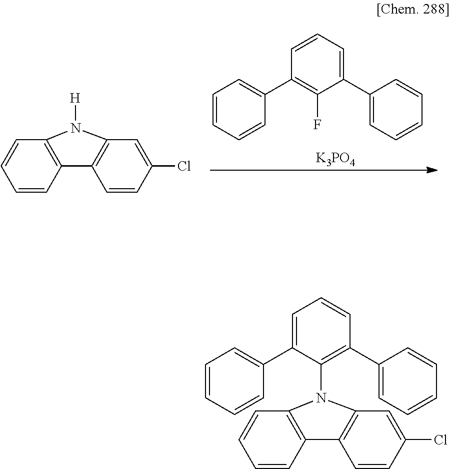

8.2 g (40 mmol) of 2-chloro-9H-carbazole, 12 g (48 mmol) of 2′-fluoro-m-terphenyl, 17 g (80 mmol) of tripotassium phosphate and 200 mL of dimethyl sulfoxide were added to a 500-mL three-neck flask and were stirred at 180° C. for 16 hours. After cooling to room temperature, 200 mL of pure water was added and stirred to precipitate a solid. The solid was then filtered off and washed with water and hexane. The solid was recrystallized with a mixed solvent of toluene and butanol to isolate 14 g (33 mmol) of 2-chloro-9-(m-terphenyl-2′-yl)carbazole as a white solid (yield: 79%).

The compound was identified by ¹H NMR measurement.

¹H-NMR (CDCl₃); 7.83 (d, J=8.0, 1H), 7.76 (d, J=8.0, 1H), 7.66-7.72 (m, 1H), 7.59-7.64 (m, 2H), 7.13-7.19 (m, 1H), 7.04-7.09 (m, 1H), 6.88-7.02 (m, 13H)

### Synthesis Example 6 (Synthesis of 2-chloro-9-(2-(2-naphthalenyl)phenyl)carbazole)

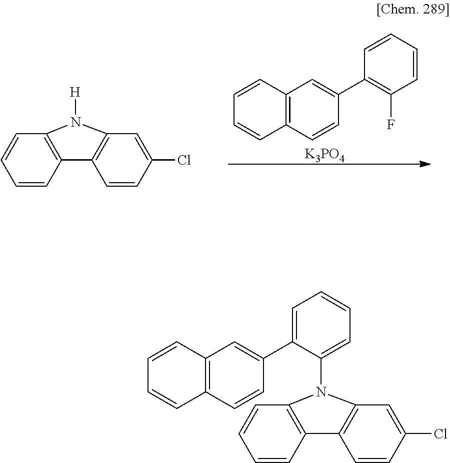

8.2 g (40 mmol) of 2-chloro-9H-carbazole, 11 g (48 mmol) of 2-(2-naphthalenyl)fluorobenzene, 17 g (80 mmol) of tripotassium phosphate and 200 mL of dimethyl sulfoxide were added to a 500-mL three-neck flask and were stirred at 180° C. for 16 hours. After cooling to room temperature, 200 m L of pure water was added and stirred to precipitate a solid. The solid was then filtered off and washed with water and hexane. The solid was recrystallized with a mixed solvent of toluene and butanol to isolate 14 g (34 mmol) of 2-chloro-9-(2-(2-naphthalenyl)phenyl)carbazole as a white solid (yield: 86%).

The compound was identified by ¹H NMR measurement.

¹H-NMR (CDCl₃); 7.92-7.99 (m, 1H), 7.86-7.91 (m, 1H), 7.75-7.80 (m, 1H), 7.47-7.70 (m, 6H), 7.28-7.42 (m, 3H), 7.21-7.28 (m, 1H), 7.07-7.17 (m, 4H), 6.96-7.04 (m, 1H)

### Synthesis Example 7 (Synthesis of 2-chloro-9-(m-terphenyl-5′-yl)carbazole)

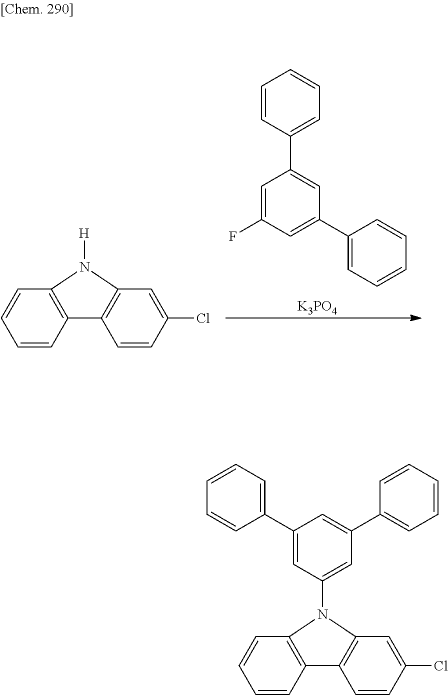

8.2 g (40 mmol) of 2-chloro-9H-carbazole, 12 g (48 mmol) of 5′-fluoro-m-terphenyl, 17 g (80 mmol) of tripotassium phosphate and 200 mL of dimethyl sulfoxide were added to a 500-mL three-neck flask and were stirred at 180° C. for 16 hours. After cooling to room temperature, 200 mL of pure water was added and stirred to precipitate a solid. The solid was then filtered off and washed with water and hexane. The solid was recrystallized with a mixed solvent of toluene and butanol to isolate 15 g (35 mmol) of 2-chloro-9-(m-terphenyl-5′-yl)carbazole as a white solid (yield: 85%).

The compound was identified by ¹H NMR measurement.

¹H-NMR (CDCl₃); 8.13 (d, J=8.0, 1H), 8.06 (d, J=8.0, 1H), 7.91-7.96 (m, 1H), 7.67-7.75 (m, 6H), 7.34-7.55 (m, 9H), 7.27-7.36 (m, 2H)

### Synthesis Example 8 (Synthesis of 2-chloro-9-(p-terphenyl-2′-yl)carbazole)

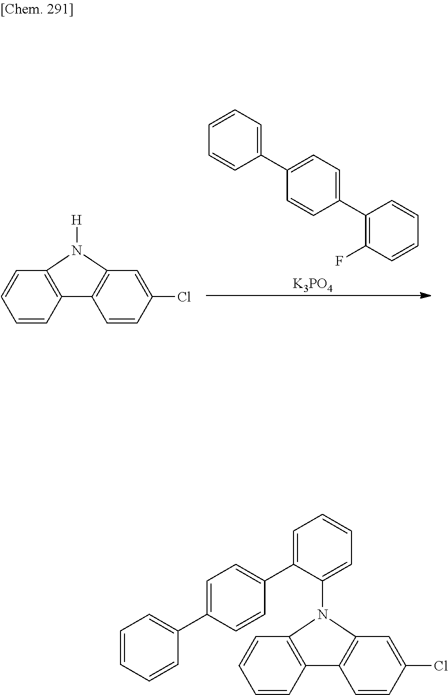 [SMILES: Clc1ccc2c(c1)[nH]c1ccccc12 ; Fc1ccccc1-c1ccc(-c2ccccc2)cc1 ; "K3PO4" ; Clc1ccc2c3ccccc3n(-c3ccccc3-c3ccc(-c4ccccc4)cc3)c2c1]

8.2 g (40 mmol) of 2-chloro-9H-carbazole, 12 g (48 mmol) of 2′-fluoro-p-terphenyl, 17 g (80 mmol) of tripotassium phosphate and 200 mL of dimethyl sulfoxide were added to a 500-mL three-neck flask and were stirred at 180° C. for 16 hours. After cooling to room temperature, 200 mL of pure water was added and stirred to precipitate a solid. The solid was then filtered off and washed with water and hexane. The solid was recrystallized with a mixed solvent of toluene and butanol to isolate 15 g (35 mmol) of 2-chloro-9-(p-terphenyl-2′-yl)carbazole as a white solid (yield: 85%).

The compound was identified by FDMS measurement.

FDMS: 429

### Synthesis Example 9 (Synthesis of 22-chloro-9-(dibenzo[b,d]thiophen-4-yl)-9H-carbazole)

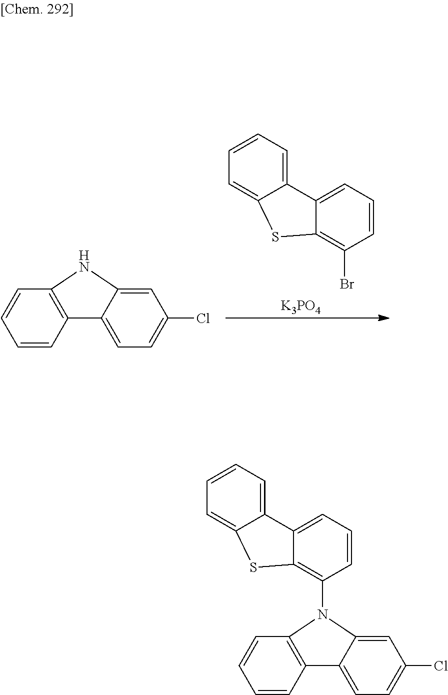 [SMILES: Clc1ccc2c(c1)[nH]c1ccccc12 ; "K3PO4" ; Brc1cccc2c1sc1ccccc12 ; Clc1ccc2c3ccccc3n(-c3cccc4c3sc3ccccc34)c2c1]

In a nitrogen stream, 3.0 g (15 mmol) of 2-chloro-9H-carbazole, 4.7 g (18 mmol) of 4-bromodibenzo[b,d]thiophene, 2.9 g (21 mmol) of potassium carbonate, 20 mL of xylene, 33 mg (0.15 mmol) of palladium acetate and 0.24 g (0.30 mmol) of a 25% by weight xylene solution of tri(tert-butyl)phosphine were added to a 100-mL three-neck flask and were stirred at 140° C. for 22 hours. After cooling to room temperature, 200 mL of pure water was added and stirred to precipitate a solid. The solid was then filtered off and washed with water and hexane. The solid was recrystallized with a mixed solvent of toluene and butanol to isolate 4.8 g (12 mmol) of 2-chloro-9-(dibenzo[b,d]thiophen-4-yl)-9H-carbazole as a white solid (yield: 84%).

The compound was identified by FDMS measurement.

FDMS: 383

### Synthesis Example 10 (Synthesis of 9-([1,1′:2′,1″-terphenyl]-2-yl)-2-chloro-9H-carbazole)

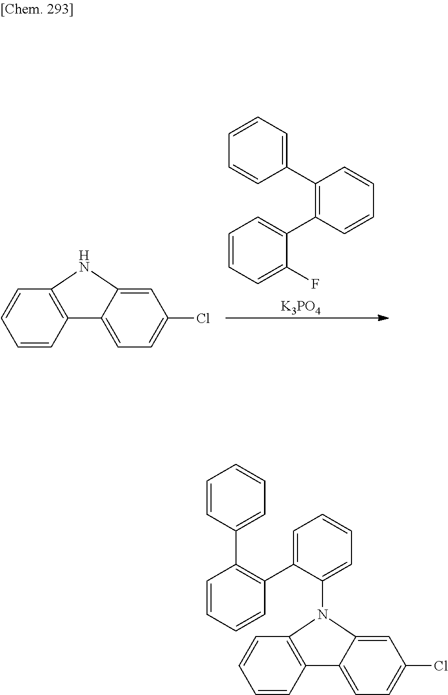

7.0 g (35 mmol) of 2-chloro-9H-carbazole, 10 g (42 mmol) of 2-fluoro-1,1′:2′,1″-terphenyl, 15 g (69 mmol) of tripotassium phosphate and 100 mL of dimethyl sulfoxide were added to a 300-mL three-neck flask and were stirred at 180° C. for 16 hours. After cooling to room temperature, 200 mL of pure water was added and stirred to precipitate a solid. The solid was then filtered off and washed with water and hexane. The solid was recrystallized with a mixed solvent of toluene and butanol to isolate 12 g (28 mmol) of 9-([1,1′:2′,1″-terphenyl]-2-yl)-2-chloro-9H-carbazole as a white solid (yield: 80%).

The compound was identified by FDMS measurement.

FDMS: 423

### Synthesis Example 11 (Synthesis of 2-chloro-9-(2-(dibenzo[b,d]furan-4-yl)phenyl)-9H-carbazole)

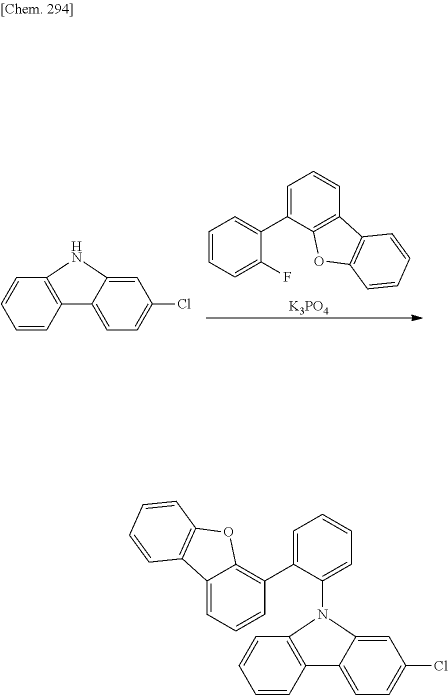

8.5 g (42 mmol) of 2-chloro-9H-carbazole, 13 g (51 mmol) of 4-(2-fluorophenyl)dibenzo[b,d]furan, 18 g (84 mmol) of tripotassium phosphate and 100 mL of dimethyl sulfoxide were added to a 300-mL three-neck flask and were stirred at 180° C. for 15 hours. After cooling to room temperature, 150 mL of pure water was added and stirred to precipitate a solid. The solid was then filtered off and washed with water and hexane. The solid was recrystallized with a mixed solvent of toluene and butanol to isolate 14 g (32 mmol) of 2-chloro-9-(2-(dibenzo[b,d]furan-4-yl)phenyl)-9H-carbazole as a white solid (yield: 76%).

The compound was identified by FDMS measurement.

FDMS: 423

### Synthesis Example 12 (Synthesis of 2-chloro-9-(2-(phenanthren-9-yl)phenyl)-9H-carbazole)

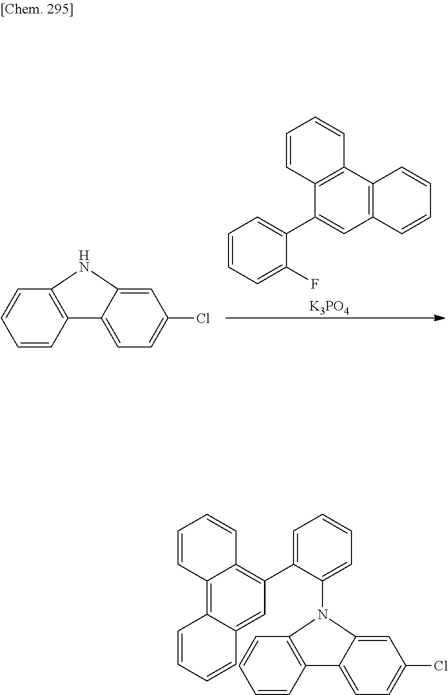

4.0 g (20 mmol) of 2-chloro-9H-carbazole, 6.5 g (24 mmol) of 9-(2-fluorophenyl)phenanthrene, 8.4 g (40 mmol) of tripotassium phosphate and 120 mL of dimethyl sulfoxide were added to a 300-mL three-neck flask and were stirred at 180° C. for 12 hours. After cooling to room temperature, 150 m L of pure water was added and stirred to precipitate a solid. The solid was then filtered off and washed with water and hexane. The solid was recrystallized with a mixed solvent of toluene and butanol to isolate 5.9 g (13 mmol) of 2-chloro-9-(2-(phenanthren-9-yl)phenyl)-9H-carbazole as a white solid (yield: 65%).

The compound was identified by FDMS measurement.

FDMS: 454

### Synthesis Example 13 (Synthesis of 4,4′-(2-fluoro-1,4-phenylene)didibenzo[b,d]furan)

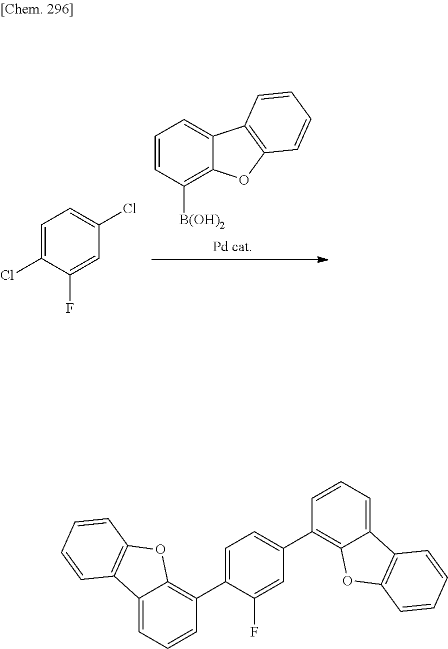 [SMILES: Fc1cc(Cl)ccc1Cl ; OB(O)c1cccc2c1oc1ccccc12 ; Fc1cc(-c2cccc3c2oc2ccccc23)ccc1-c1cccc2c1oc1ccccc12]

In a nitrogen stream, 5.0 g (30 mmol) of 1,4-dichloro-2-fluorobenzene, 15 g (70 mmol) of dibenzo[b,d]furan-4-ylboronic acid, 68 mg (0.30 mmol) of palladium acetate, 0.29 g (0.61 mmol) of XPhos and 100 mL of THE were added to a 200-mL three-neck flask and were stirred at 60° C. 15 mL (61 mmol) of 2 M aqueous tripotassium phosphate was added dropwise thereto and was stirred at 70° C. for 22 hours. After cooling to room temperature, the product was transferred to a separatory funnel, and an organic layer was extracted with toluene. The organic layer was then washed with saturated aqueous sodium chloride. The organic layer was dried over anhydrous magnesium sulfate and was then subjected to column chromatography with a small amount of silica gel to remove a polar component. The solvent was then distilled off under reduced pressure, and the resulting solid was recrystallized with a mixed solvent of toluene and butanol to isolate 9.9 g (23 mmol) of 4,4′-(2-fluoro-1,4-phenylene)didibenzo[b,d]furan as a white solid (yield: 76%).

The compound was identified by FDMS measurement.

FDMS: 428

### Synthesis Example 14 (Synthesis of 2,5-bis(dibenzo[b,d]furan-4-yl)phenyl)-2-chloro-9H-carbazole)

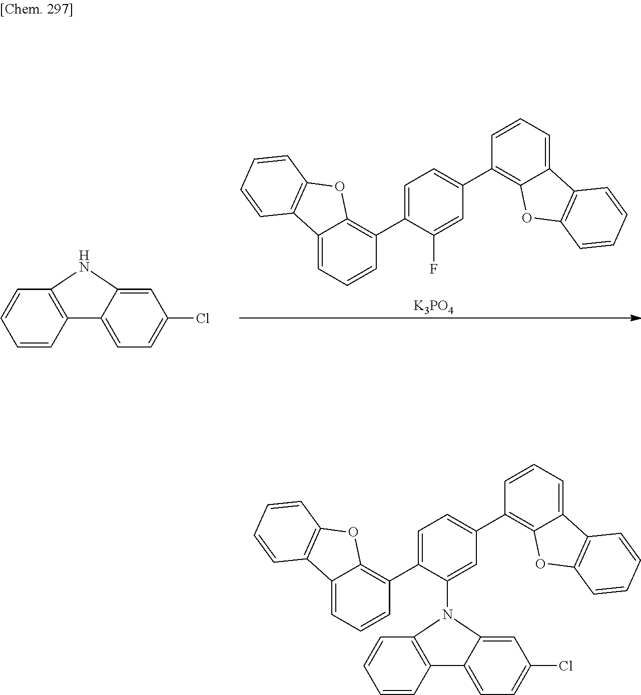

3.0 g (15 mmol) of 2-chloro-9H-carbazole, 7.6 g (18 mmol) of 4,4′-(2-fluoro-1,4-phenylene)didibenzo[b,d]furan prepared in Synthesis Example 14, 3.2 g (15 mmol) of tripotassium phosphate and 60 mL of dimethyl sulfoxide were added to a 300-mL three-neck flask and were stirred at 180° C. for 15 hours. After cooling to room temperature, 150 mL of pure water was added and stirred to precipitate a solid. The solid was then filtered off and washed with water and hexane. The solid was recrystallized with a mixed solvent of toluene and butanol to isolate 6.8 g (11 mmol) of 9-(2,5-bis(dibenzo[b,d]furan-4-yl)phenyl)-2-chloro-9H-carbazole as a white solid (yield: 75%).

The compound was identified by FDMS measurement.

FDMS: 609

### Synthesis Example 15 (Synthesis of 2-(6-fluoro-[1,1′-biphenyl]-2-yl)naphthalene)

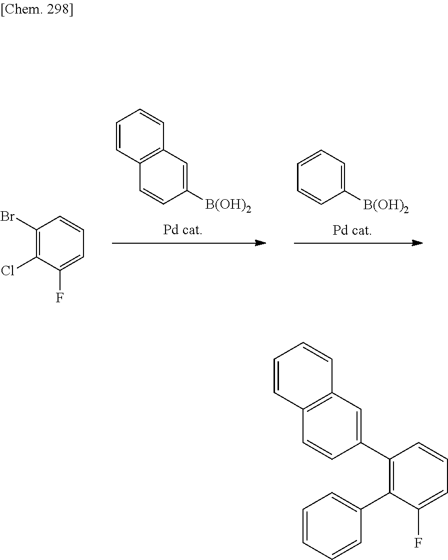 [SMILES: OB(O)c1ccc2ccccc2c1 ; OB(O)c1ccccc1 ; Fc1cccc(Br)c1Cl ; Fc1cccc(-c2ccc3ccccc3c2)c1-c1ccccc1]

In a nitrogen stream, 10 g (48 mmol) of 1-bromo-2-chloro-3-fluorobenzene, 8.2 g (48 mmol) of naphthalen-2-ylboronic acid, 0.11 g (0.48 mmol) of palladium acetate, 0.53 g (0.95 mmol) of 1,1′-bis(diphenylphosphino)ferrocene and 100 mL of THE were added to a 100-mL three-neck flask and were stirred at 60° C. 24 mL (95 mmol) of 4 M aqueous potassium carbonate was added dropwise and stirred at 70° C. for 22 hours. After cooling to room temperature, the product was transferred to a separatory funnel, and an organic layer was extracted with toluene. The organic layer was then washed with saturated aqueous sodium chloride. The organic layer was dried over anhydrous magnesium sulfate and was then subjected to column chromatography with a small amount of silica gel to remove a polar component. The solvent was then distilled off under reduced pressure to prepare an oil. 5.8 g (48 mmol) of phenylboronic acid, 0.11 g (0.48 mmol) of palladium acetate, 0.46 g (0.95 mmol) of XPhos and 100 mL of THE were then added to a 100-mL three-neck flask in a nitrogen stream and were stirred at 60° C. 24 mL (95 mmol) of 4 M aqueous tripotassium phosphate was added dropwise thereto and was stirred at 70° C. for 16 hours. After cooling to room temperature, the product was transferred to a separatory funnel, and an organic layer was extracted with toluene. The organic layer was then washed with saturated aqueous sodium chloride. The organic layer was dried over anhydrous magnesium sulfate and was then subjected to column chromatography with a small amount of silica gel to remove a polar component. The solvent was then distilled off under reduced pressure, and the resulting solid was recrystallized with a mixed solvent of toluene and butanol to isolate 8.4 g (28 mmol) of 2-(6-fluoro-[1,1′-biphenyl]-2-yl)naphthalene as a white solid (yield: 59%).

The compound was identified by FDMS measurement.

FDMS: 298

### Synthesis Example 16 (Synthesis of 2-chloro-9-(6-(naphthalen-2-yl)-[1,1′-biphenyl]-2-yl)-9H-carbazole)

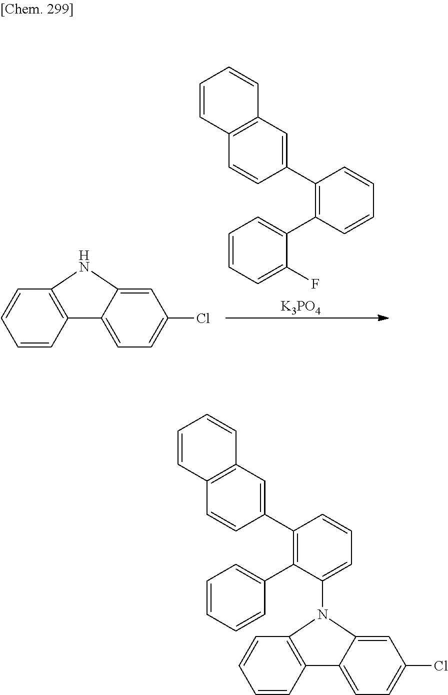

2.5 g (12 mmol) of 2-chloro-9H-carbazole, 4.4 g (15 mmol) of 2-(6-fluoro-[1,1′-biphenyl]-2-yl)naphthalene prepared in Synthesis Example 15, 2.6 g (12 mmol) of tripotassium phosphate and 60 mL of dimethyl sulfoxide were added to a 300-mL three-neck flask and were stirred at 180° C. for 30 hours. After cooling to room temperature, 100 mL of pure water was added thereto and stirred. An aqueous layer and an organic layer were then separated, and the organic layer was washed with saturated aqueous sodium chloride. The organic layer was dried over anhydrous magnesium sulfate and was then subjected to column chromatography with a small amount of silica gel to remove a polar component. The solvent was then distilled off under reduced pressure, and the resulting solid was recrystallized with a mixed solvent of toluene and butanol to isolate 4.8 g (9.9 mmol) of 2-chloro-9-(6-(naphthalen-2-yl)-[1,1′-biphenyl]-2-yl)-9H-carbazole as a white solid (yield: 80%).

The compound was identified by FDMS measurement.

FDMS: 479

### Synthesis Example 17 (Synthesis of 2-chloro-9-(2-(phenanthren-9-yl)phenyl)-9H-carbazole)

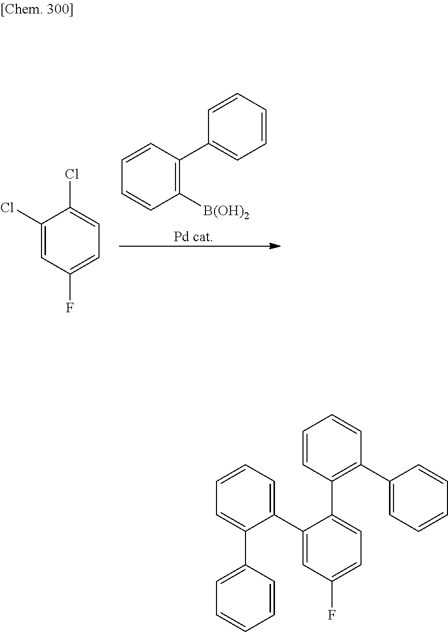

In a nitrogen stream, 5.0 g (30 mmol) of 1,2-dichloro-4-fluorobenzene, 14 g (73 mmol) of [1,1′-biphenyl]-2-ylboronic acid, 68 mg (0.30 mmol) of palladium acetate, 0.29 g (0.61 mmol) of XPhos and 100 mL of THE were added to a 100-mL three-neck flask and were stirred at 60° C. 15 mL (61 mmol) of 4 M aqueous tripotassium phosphate was added dropwise thereto and was stirred at 70° C. for 22 hours. After cooling to room temperature, 100 mL of pure water was added thereto and stirred. An aqueous layer and an organic layer were then separated, and the organic layer was washed with saturated aqueous sodium chloride. The organic layer was dried over anhydrous magnesium sulfate and was then subjected to column chromatography with a small amount of silica gel to remove a polar component. The solvent was then distilled off under reduced pressure, and the resulting solid was recrystallized with a mixed solvent of toluene and butanol to isolate 7.8 g (19 mmol) of 4″-fluoro-1,1′:2′,1″:2″,1′″:2′″,1″″-quinquiphenyl as a white solid (yield: 64%).

The compound was identified by FDMS measurement.

FDMS: 400

### Synthesis Example 18 (Synthesis of 9-([1,1′:2′,1″:2″,1′″:2′″,1″″-quinquiphenyl]-4″-yl)-2-chloro-9H-carbazole)

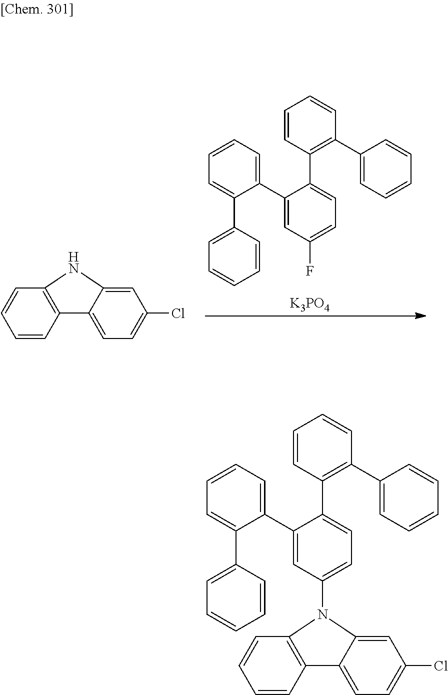

2.0 g (9.9 mmol) of 2-chloro-9H-carbazole, 4.8 g (12 mmol) of 4″-fluoro-1,1′:2′,1″:2″,1′″:2′″,1″″-quinquiphenyl prepared in Synthesis Example 17, 2.1 g (9.9 mmol) of tripotassium phosphate and 50 mL of dimethyl sulfoxide were added to a 300-mL three-neck flask and were stirred at 180° C. for 40 hours. After cooling to room temperature, 150 mL of pure water was added and stirred to precipitate a solid. The solid was then filtered off and washed with water and hexane. The solid was recrystallized with a mixed solvent of toluene and butanol to isolate 4.6 g (7.9 mmol) of 9-([1,1′:2′,1″:2″,1′″:2′″,1″″-quinquiphenyl]-4″-yl)-2-chloro-9H-carbazole as a white solid (yield: 80%).

The compound was identified by FDMS measurement.

FDMS: 582

### Synthesis Example 19 (Synthesis of 9-(2-bromo-6-methylphenyl)-2-chloro-9H-carbazole)

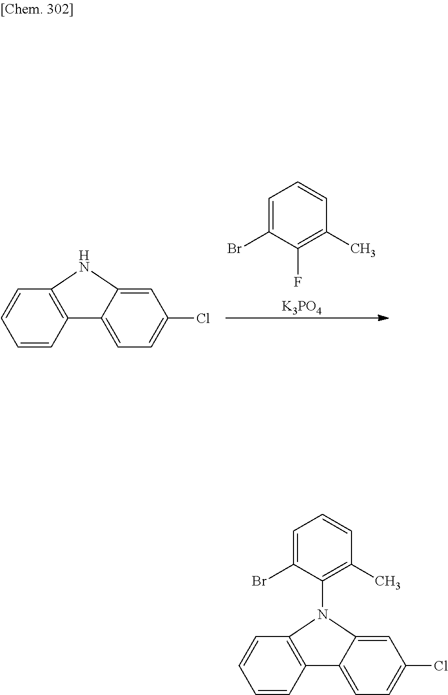

2.0 g (9.9 mmol) of 2-chloro-9H-carbazole, 2.8 g (15 mmol) of 1-bromo-2-fluoro-3-methylbenzene, 2.1 g (9.9 mmol) of tripotassium phosphate and 45 mL of dimethyl sulfoxide were added to a 300-mL three-neck flask and were stirred at 180° C. for 9 hours. After cooling to room temperature, 100 mL of pure water was added thereto and stirred. An aqueous layer and an organic layer were then separated, and the organic layer was washed with saturated aqueous sodium chloride. The organic layer was dried over anhydrous magnesium sulfate and was then subjected to column chromatography with a small amount of silica gel to remove a polar component. The solvent was then distilled off under reduced pressure, and the resulting solid was recrystallized with a mixed solvent of toluene and butanol to isolate 2.9 g (7.8 mmol) of 9-(2-bromo-6-methylphenyl)-2-chloro-9H-carbazole as a white solid (yield: 79%).

The compound was identified by FDMS measurement.

FDMS: 369

### Synthesis Example 20 (Synthesis of 2-chloro-9-(2-methyl-6-(naphthalen-2-yl)phenyl)-9H-carbazole)

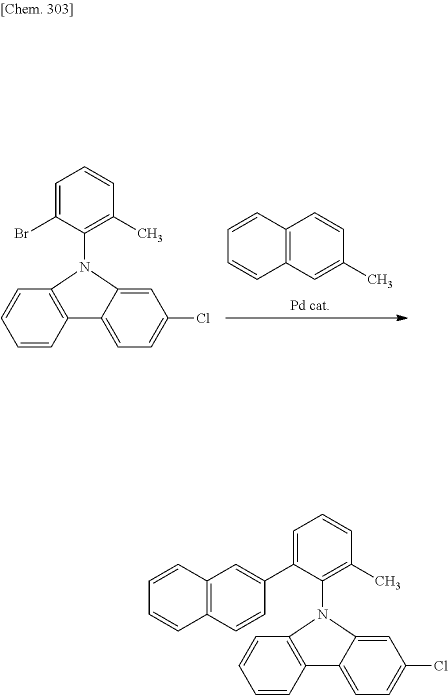

In a nitrogen stream, 2.9 g (7.8 mmol) of 9-(2-bromo-6-methylphenyl)-2-chloro-9H-carbazole prepared in Synthesis Example 19, 1.5 g (8.6 mmol) of naphthalen-2-ylboronic acid, 18 mg (78 μmol) of palladium acetate, 89 mg (0.16 mmol) of 1,1′-bis(diphenylphosphino)ferrocene and 60 mL of THE were added to a 100-mL three-neck flask and were stirred at 60° C. 3.9 mL (16 mmol) of 4 M aqueous tripotassium phosphate was added dropwise thereto and was stirred at 70° C. for 22 hours. After cooling to room temperature, the product was transferred to a separatory funnel, and an organic layer was extracted with toluene. The organic layer was then washed with saturated aqueous sodium chloride. The organic layer was dried over anhydrous magnesium sulfate and was then subjected to column chromatography with a small amount of silica gel to remove a polar component. The solvent was then distilled off under reduced pressure, and the resulting solid was recrystallized with a mixed solvent of toluene and butanol to isolate 2.9 g (7.0 mmol) of 2-chloro-9-(2-methyl-6-(naphthalen-2-yl)phenyl)-9H-carbazole as a white solid (yield: 90%).

The compound was identified by FDMS measurement.

FDMS: 417

### Synthesis Example 21 (Synthesis of 2-(2-fluoro-[1,1′-biphenyl]-3-yl)naphthalene)

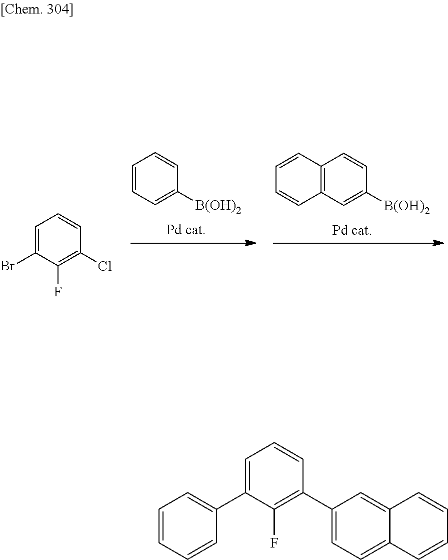 [SMILES: OB(O)c1ccccc1 ; OB(O)c1ccc2ccccc2c1 ; Fc1c(Cl)cccc1Br ; Fc1c(-c2ccccc2)cccc1-c1ccc2ccccc2c1]

In a nitrogen stream, 10. g (48 mmol) of 1-bromo-3-chloro-2-fluorobenzene, 9.0 g (53 mmol) of naphthalen-2-ylboronic acid, 0.11 g (0.48 mmol) of palladium acetate, 0.46 g (0.95 mmol) and 100 mL of THE were added to a 100-mL three-neck flask and were stirred at 60° C. 24 mL (95 mmol) of 4 M aqueous potassium carbonate was added dropwise thereto and was stirred at 70° C. for 22 hours. After cooling to room temperature, the product was transferred to a separatory funnel, and an organic layer was extracted with toluene. The organic layer was then washed with saturated aqueous sodium chloride. The organic layer was dried over anhydrous magnesium sulfate and was then subjected to column chromatography with a small amount of silica gel to remove a polar component. The solvent was then distilled off under reduced pressure to prepare an oil. 6.4 g (53 mmol) of phenylboronic acid, 0.11 g (0.48 mmol) of palladium acetate, 0.46 g (0.95 mmol) of XPhos and 100 mL of THE were then added to a 100-mL three-neck flask in a nitrogen stream and were stirred at 60° C. 24 mL (95 mmol) of 4 M aqueous tripotassium phosphate was added dropwise thereto and was stirred at 70° C. for 16 hours. After cooling to room temperature, the product was transferred to a separatory funnel, and an organic layer was extracted with toluene. The organic layer was then washed with saturated aqueous sodium chloride. The organic layer was dried over anhydrous magnesium sulfate and was then subjected to column chromatography with a small amount of silica gel to remove a polar component. The solvent was then distilled off under reduced pressure, and the resulting solid was recrystallized with a mixed solvent of toluene and butanol to isolate 10 g (34 mmol) of 2-(2-fluoro-[1,1′-biphenyl]-3-yl)naphthalene as a white solid (yield: 71%).

The compound was identified by FDMS measurement.

FDMS: 298

### Synthesis Example 22 (Synthesis of 2-chloro-9-(3-(naphthalen-2-yl)-[1,1′-biphenyl]-2-yl)-9H-carbazole)

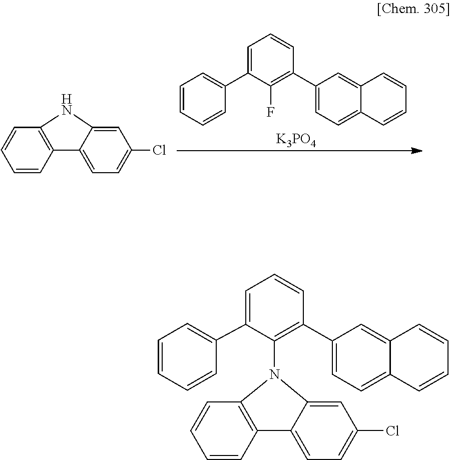

7.0 g (35 mmol) of 2-chloro-9H-carbazole, 12 g (42 mmol) of 2-(2-fluoro-[1,1′-biphenyl]-3-yl)naphthalene prepared in Synthesis Example 21, 7.4 g (35 mmol) of tripotassium phosphate and 70 mL of dimethyl sulfoxide were added to a 300-mL three-neck flask and were stirred at 180° C. for 12 hours. After cooling to room temperature, 150 mL of pure water was added and stirred to precipitate a solid. The solid was then filtered off and washed with water and hexane. The solid was recrystallized with a mixed solvent of toluene and butanol to isolate 14 g (30 mmol) of 2-chloro-9-(3-(naphthalen-2-yl)-[1,1′-biphenyl]-2-yl)-9H-carbazole as a white solid (yield: 86%).

The compound was identified by FDMS measurement.

FDMS: 479

### Synthesis Example 23 (Synthesis of 1,1′-(2-fluoro-1,3-phenylene)dinaphthalene)

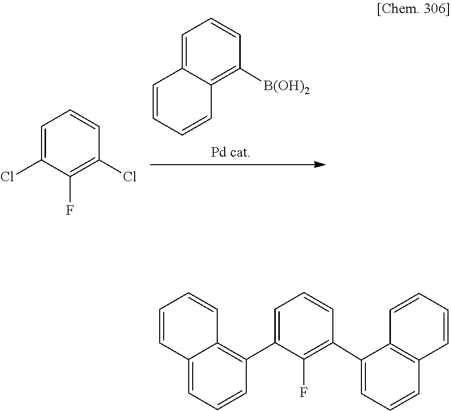 [SMILES: Fc1c(Cl)cccc1Cl ; OB(O)c1cccc2ccccc12 ; Fc1c(-c2cccc3ccccc23)cccc1-c1cccc2ccccc12]

In a nitrogen stream, 5.0 g (30 mmol) of 1,3-dichloro-2-fluorobenzene, 6.3 g (36 mmol) of naphthalen-1-ylboronic acid, 68 mg (0.30 mmol) of palladium acetate, 0.29 g (0.61 mmol) of XPhos and 100 mL of THE were added to a 100-mL three-neck flask and were stirred at 60° C. 15 mL (61 mmol) of 4 M aqueous tripotassium phosphate was added dropwise thereto and was stirred at 70° C. for 22 hours. After cooling to room temperature, the product was transferred to a separatory funnel, and an organic layer was extracted with toluene. The organic layer was then washed with saturated aqueous sodium chloride. The organic layer was dried over anhydrous magnesium sulfate and was then subjected to column chromatography with a small amount of silica gel to remove a polar component. The solvent was then distilled off under reduced pressure, and the resulting solid was recrystallized with a mixed solvent of toluene and butanol to isolate 9.9 g (27 mmol) of 9.5 g (27 mmol) of 1,1′-(2-fluoro-1,3-phenylene)dinaphthalene as a white solid (yield: 90%).

The compound was identified by FDMS measurement.

FDMS: 348

### Synthesis Example 24 (Synthesis of 2-chloro-9-(2,6-di(naphthalen-1-yl)phenyl)-9H-carbazole)

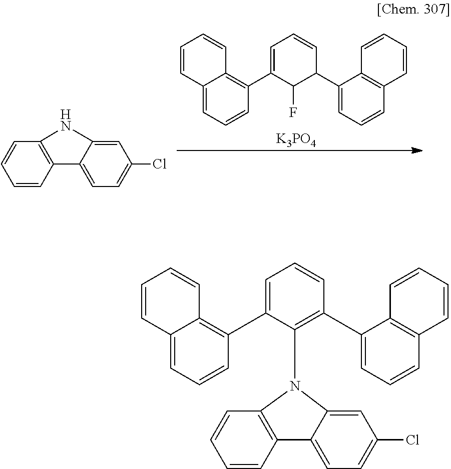

7.0 g (35 mmol) of 2-chloro-9H-carbazole, 15 g (42 mmol) of 1,1′-(2-fluoro-1,3-phenylene)dinaphthalene prepared in Synthesis Example 23, 7.4 g (35 mmol) of tripotassium phosphate and 100 mL of dimethyl sulfoxide were added to a 300-mL three-neck flask and were stirred at 180° C. for 15 hours. After cooling to room temperature, 150 mL of pure water was added and stirred to precipitate a solid. The solid was then filtered off and washed with water and hexane. The solid was recrystallized with a mixed solvent of toluene and butanol to isolate 15 g (29 mmol) of 2-chloro-9-(2,6-di(naphthalen-1-yl)phenyl)-9H-carbazole as a white solid (yield: 83%).

The compound was identified by FDMS measurement.

FDMS: 529

### Synthesis Example 25 (Synthesis of 2′-fluoro-3,3″-dimethyl-1,1′:3′,1″-terphenyl)

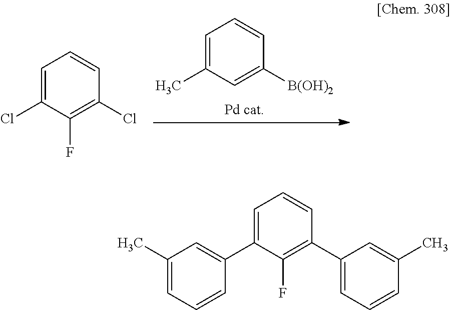

In a nitrogen stream, 5.0 g (30 mmol) of 1,3-dichloro-2-fluorobenzene, 4.5 g (33 mmol) of m-tolylboronic acid, 68 mg (0.30 mmol) of palladium acetate, 0.29 g (0.61 mmol) of XPhos and 100 mL of THE were added to a 100-mL three-neck flask and were stirred at 60° C. 15 mL (61 mmol) of 4 M aqueous tripotassium phosphate was added dropwise thereto and was stirred at 70° C. for 15 hours. After cooling to room temperature, the product was transferred to a separatory funnel, and an organic layer was extracted with toluene. The organic layer was then washed with saturated aqueous sodium chloride. The organic layer was dried over anhydrous magnesium sulfate and was then subjected to column chromatography with a small amount of silica gel to remove a polar component. The solvent was then distilled off under reduced pressure, and the resulting solid was recrystallized with a mixed solvent of toluene and butanol to isolate 6.7 g (24 mmol) of 2′-fluoro-3,3″-dimethyl-1,1′:3′,1″-terphenyl as a white solid (yield: 80%).

The compound was identified by FDMS measurement.

FDMS: 276

### Synthesis Example 26 (Synthesis of 2-chloro-9-(3,3″-dimethyl-[1,1′:3′,1″-terphenyl]-2′-yl)-9H-carbazole)

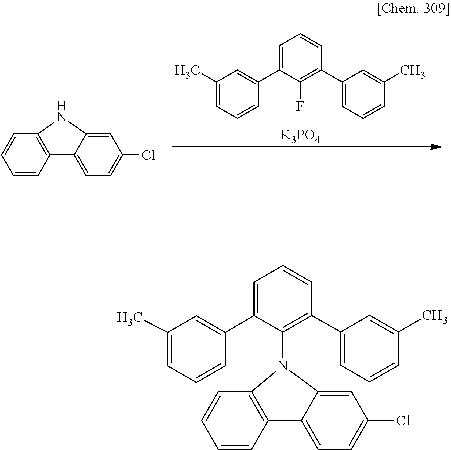

3.0 g (15 mmol) of 2-chloro-9H-carbazole, 4.9 g (18 mmol) of 2′-fluoro-3,3″-dimethyl-1,1:3′,1″-terphenyl prepared in Synthesis Example 25, 3.2 g (15 mmol) of tripotassium phosphate and 30 mL of dimethyl sulfoxide were added to a 300-mL three-neck flask and were stirred at 180° C. for 15 hours. After cooling to room temperature, 100 mL of pure water was added and stirred to precipitate a solid. The solid was then filtered off and washed with water and hexane. The solid was recrystallized with a mixed solvent of toluene and butanol to isolate 5.2 g (11 mmol) of 2-chloro-9-(3,3″-dimethyl-[1,1′:3′,1″-terphenyl]-2′-yl)-9H-carbazole as a white solid (yield: 77%).

The compound was identified by FDMS measurement.

FDMS: 457

### Synthesis Example 27 (Synthesis of 4-(2′-fluoro-5′-methyl-[1,1′-biphenyl]-2-yl)dibenzo[b,d]furan)

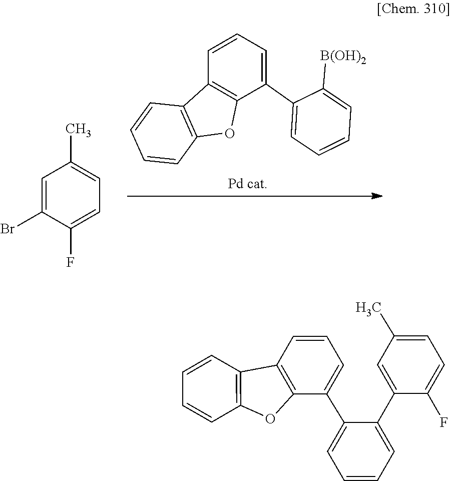

In a nitrogen stream, 5.0 g (26 mmol) of 2-bromo-1-fluoro-4-methylbenzene, 8.4 g (29 mmol) of (2-(dibenzo[b,d]furan-4-yl)phenyl)boronic acid, 59 mg (0.26 mmol) of palladium acetate, 0.25 g (0.53 mmol) of XPhos and 100 mL of THE were added to a 100-mL three-neck flask and were stirred at 60° C. 13 mL (53 mmol) of 4 M aqueous tripotassium phosphate was added dropwise thereto and was stirred at 70° C. for 16 hours. After cooling to room temperature, the product was transferred to a separatory funnel, and an organic layer was extracted with toluene. The organic layer was then washed with saturated aqueous sodium chloride. The organic layer was dried over anhydrous magnesium sulfate and was then subjected to column chromatography with a small amount of silica gel to remove a polar component. The solvent was then distilled off under reduced pressure, and the resulting solid was recrystallized with a mixed solvent of toluene and butanol to isolate 8.1 g (23 mmol) of 4-(2′-fluoro-5′-methyl-[1,1′-biphenyl]-2-yl)dibenzo[b,d]furan as a white solid (yield: 87%).

The compound was identified by FDMS measurement.

FDMS: 382

### Synthesis Example 28 (Synthesis of 9-(2′-(dibenzo[b,d]furan-4-yl)-5-methyl-[1,1′-biphenyl]-2-yl)-9H-carbazole)

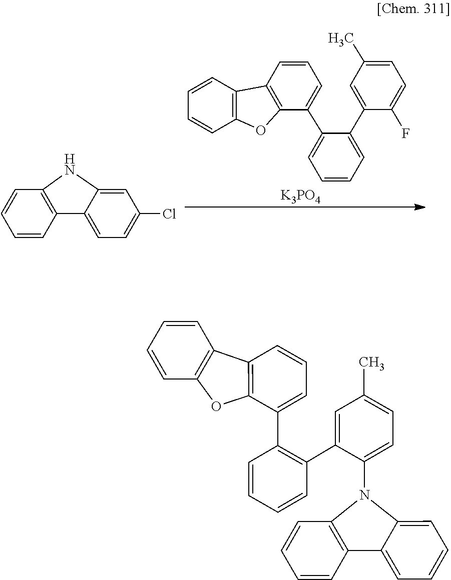

2.7 g (13 mmol) of 2-chloro-9H-carbazole, 5.7 g (16 mmol) of 4-(2′-fluoro-5′-methyl-[1,1′-biphenyl]-2-yl)dibenzo[b,d]furan prepared in Synthesis Example 27, 2.8 g (13 mmol) of tripotassium phosphate and 30 mL of dimethyl sulfoxide were added to a 300-mL three-neck flask and were stirred at 180° C. for 40 hours. After cooling to room temperature, 150 mL of pure water was added and stirred to precipitate a solid. The solid was then filtered off and washed with water and hexane. The solid was recrystallized with a mixed solvent of toluene and butanol to isolate 5.0 g (9.9 mmol) 9-(2′-(dibenzo[b,d]furan-4-yl)-5-methyl-[1,1′-biphenyl]-2-yl)-9H-carbazole as a white solid (yield: 74%).

The compound was identified by FDMS measurement.

FDMS: 499

### Synthesis Example 29 (Synthesis of 2-chloro-9-(3′,5′-dimethyl-[1,1′-biphenyl]-2-yl)-9H-carbazole)

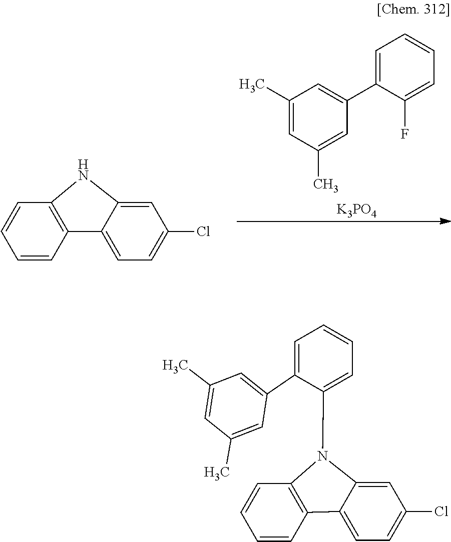

2.7 g (13 mmol) of 2-chloro-9H-carbazole, 3.2 g (16 mmol) of 2-fluoro-3′,5′-dimethyl-1,1′-biphenyl, 5.7 g (27 mmol) of tripotassium phosphate and 30 mL of dimethyl sulfoxide were added to a 300-mL three-neck flask and were stirred at 180° C. for 30 hours. After cooling to room temperature, 100 mL of pure water was added thereto and stirred. An aqueous layer and an organic layer were then separated, and the organic layer was washed with saturated aqueous sodium chloride. The organic layer was dried over anhydrous magnesium sulfate and was then subjected to column chromatography with a small amount of silica gel to remove a polar component. The solvent was then distilled off under reduced pressure, and the resulting solid was recrystallized with a mixed solvent of toluene and butanol to isolate 3.6 g (9.5 mmol) of 2-chloro-9-(3′,5′-dimethyl-[1,1′-biphenyl]-2-yl)-9H-carbazole as a white solid (yield: 71%).

The compound was identified by FDMS measurement.

FDMS: 381

### Synthesis Example 30 (Synthesis of 2-chloro-9-(2′-methyl-[1,1′-biphenyl]-2-yl)-9H-carbazole)

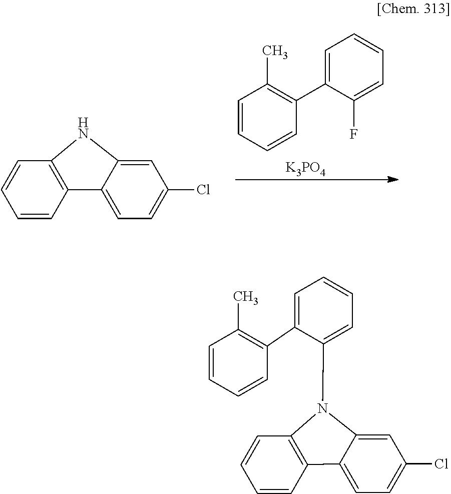

2.7 g (13 mmol) of 2-chloro-9H-carbazole, 3.0 g (16 mmol) of 2-fluoro-2′-methyl-1,1′-biphenyl, 5.7 g (27 mmol) of tripotassium phosphate and 100 mL of dimethyl sulfoxide were added to a 300-mL three-neck flask and were stirred at 180° C. for 15 hours. After cooling to room temperature, 150 mL of pure water was added and stirred to precipitate a solid. The solid was then filtered off and washed with water and hexane. The solid was recrystallized with a mixed solvent of toluene and butanol to isolate 3.4 g (9.4 mmol) of 2-chloro-9-(2′-methyl-[1,1′-biphenyl]-2-yl)-9H-carbazole as a white solid (yield: 70%).

The compound was identified by FDMS measurement.

FDMS: 367

### Synthesis Example 31 (Synthesis of 1-(6-fluoro-2′-methyl-[1,1′-biphenyl]-3-yl)naphthalene)

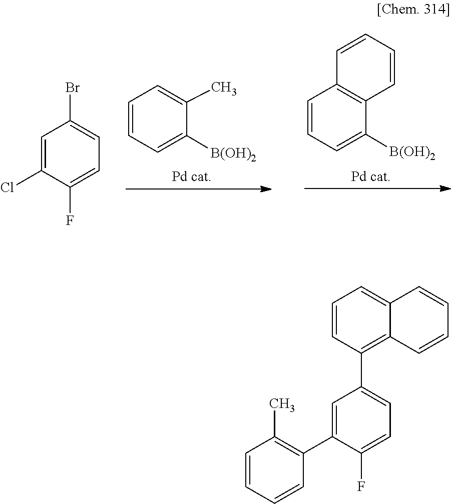

In a nitrogen stream, 5.0 g (24 mmol) of 4-bromo-2-chloro-1-fluorobenzene, 3.2 g (24 mmol) of o-tolylboronic acid, 54 mg (0.24 mmol) of palladium acetate, 0.27 g (0.48 mmol) of 1,1′-bis(diphenylphosphino)ferrocene and 75 mL of THE were added to a 100-mL three-neck flask and were stirred at 60° C. 12 mL (48 mmol) of 4 M aqueous potassium carbonate was added dropwise thereto and was stirred at 70° C. for 22 hours. After cooling to room temperature, the product was transferred to a separatory funnel, and an organic layer was extracted with toluene. The organic layer was then washed with saturated aqueous sodium chloride. The organic layer was dried over anhydrous magnesium sulfate and was then subjected to column chromatography with a small amount of silica gel to remove a polar component. The solvent was then distilled off under reduced pressure to prepare an oil. 4.1 g (24 mmol) of naphthalen-1-ylboronic acid, 54 mg (0.24 mmol) of palladium acetate, 0.23 g (0.48 mmol) of XPhos and 80 mL of THE were then added to a 100-mL three-neck flask in a nitrogen stream and were stirred at 60° C. 12 mL (48 mmol) of 4 M aqueous tripotassium phosphate was added dropwise thereto and was stirred at 70° C. for 20 hours. After cooling to room temperature, the product was transferred to a separatory funnel, and an organic layer was extracted with toluene. The organic layer was then washed with saturated aqueous sodium chloride. The organic layer was dried over anhydrous magnesium sulfate and was then subjected to column chromatography with a small amount of silica gel to remove a polar component. The solvent was then distilled off under reduced pressure, and the resulting solid was recrystallized with a mixed solvent of toluene and butanol to isolate 5.8 g (19 mmol) of 1-(6-fluoro-2′-methyl-[1,1′-biphenyl]-3-yl)naphthalene as a white solid (yield: 78%).

The compound was identified by FDMS measurement.

FDMS: 312

### Synthesis Example 32 (Synthesis of 2-chloro-9-methyl-9-(2′-methyl-5-(naphthalen-1-yl)-[1,1′-biphenyl]-2-yl)-9H-9I4-carbazole)

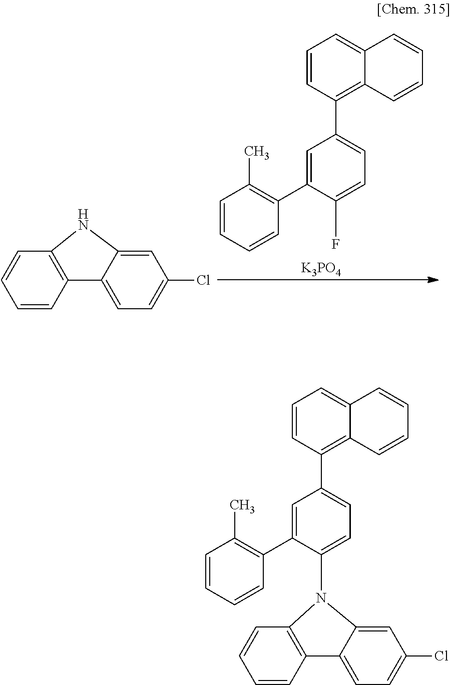

4.0 g (20 mmol) of 2-chloro-9H-carbazole, 7.4 g (24 mmol) 1-(6-fluoro-2′-methyl-[1,1′-biphenyl]-3-yl)naphthalene prepared in Synthesis Example 31, 8.4 g (40 mmol) of tripotassium phosphate and 40 mL of dimethyl sulfoxide were added to a 300-mL three-neck flask and were stirred at 180° C. for 28 hours. After cooling to room temperature, 150 mL of pure water was added and stirred to precipitate a solid. The solid was then filtered off and washed with water and hexane. The solid was recrystallized with a mixed solvent of toluene and butanol to isolate 6.7 g (13 mmol) of 2-chloro-9-methyl-9-(2′-methyl-5-(naphthalen-1-yl)-[1,1′-biphenyl]-2-yl)-9H-9I4-carbazole as a white solid (yield: 66%).

The compound was identified by FDMS measurement.

FDMS: 508

### Synthesis Example 33 (Synthesis of 4-chloro-9-(2′-(naphthalen-1-yl)-[1,1′-biphenyl]-4-yl)-9H-carbazole)

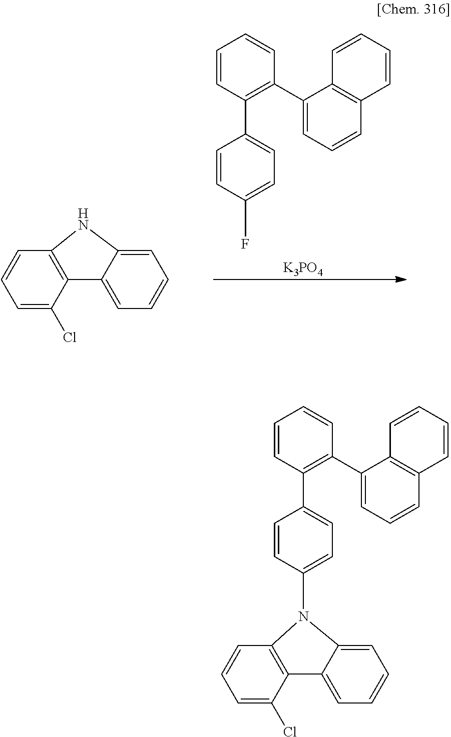

4.1 g (20 mmol) of 2-chloro-9H-carbazole, 7.3 g (24 mmol) of 1-(4′-fluoro-[1,1′-biphenyl]-2-yl)naphthalene, 8.6 g (41 mmol) of tripotassium phosphate and 50 mL of dimethyl sulfoxide were added to a 300-mL three-neck flask and were stirred at 180° C. for 35 hours. After cooling to room temperature, 150 m L of pure water was added and stirred to precipitate a solid. The solid was then filtered off and washed with water and hexane. The solid was recrystallized with a mixed solvent of toluene and butanol to isolate 8.3 g (17 mmol) of 4-chloro-9-(2′-(naphthalen-1-yl)-[1,1′-biphenyl]-4-yl)-9H-carbazole as a white solid (yield: 85%).

The compound was identified by FDMS measurement.

FDMS: 479

### Synthesis Example 34 (Synthesis of 4-chloro-9-(2-(dibenzo[b,d]thiophen-4-yl)phenyl)-9H-carbazole)

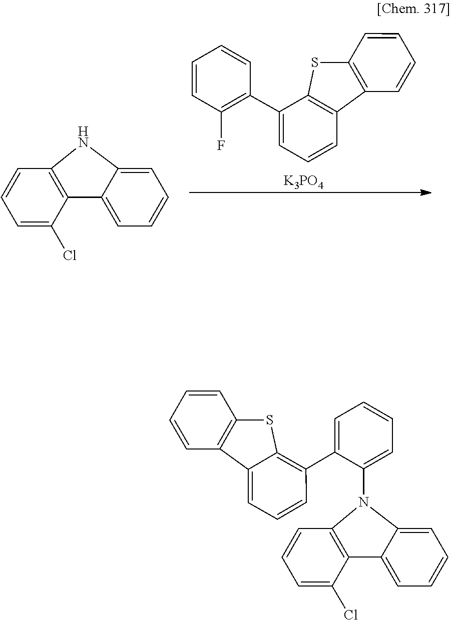

4.5 g (22 mmol) of 2-chloro-9H-carbazole, 7.5 g (27 mmol) of 4-(2-fluorophenyl)dibenzo[b,d]thiophene, 9.5 g (45 mmol) of tripotassium phosphate and 50 mL of dimethyl sulfoxide were added to a 300-mL three-neck flask and were stirred at 180° C. for 15 hours. After cooling to room temperature, 150 mL of pure water was added and stirred to precipitate a solid. The solid was then filtered off and washed with water and hexane. The solid was recrystallized with a mixed solvent of toluene and butanol to isolate 7.5 g (16 mmol) of 4-chloro-9-(2-(dibenzo[b,d]thiophen-4-yl)phenyl)-9H-carbazole as a white solid (yield: 73%).

The compound was identified by FDMS measurement.

FDMS: 459

### Synthesis Example 35 (Synthesis of 4-chloro-9-(2′-(naphthalen-2-yl)-[1,1′-biphenyl]-3-yl)-9H-carbazole)

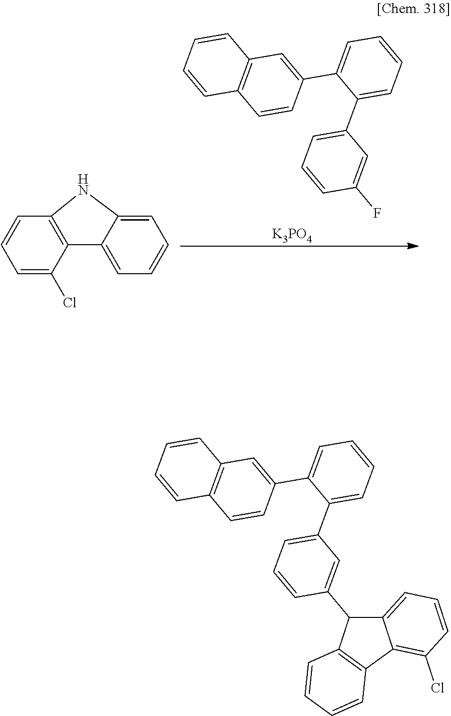 [SMILES: Clc1cccc2[nH]c3ccccc3c12 ; Fc1cccc(-c2ccccc2-c2ccc3ccccc3c2)c1 ; "K3PO4" ; Clc1cccc2c1-c1ccccc1C2c1cccc(-c2ccccc2-c2ccc3ccccc3c2)c1]

3.0 g (15 mmol) of 4-chloro-9H-carbazole, 5.3 g (18 mmol) of 2-(3′-fluoro-[1,1′-biphenyl]-2-yl)naphthalene, 6.3 g (30 mmol) of tripotassium phosphate and 30 mL of dimethyl sulfoxide were added to a 300-mL three-neck flask and were stirred at 180° C. for 35 hours. After cooling to room temperature, 150 mL of pure water was added and stirred to precipitate a solid. The solid was then filtered off and washed with water and hexane. The solid was recrystallized with a mixed solvent of toluene and butanol to isolate 5.7 g (12 mmol) of 4-chloro-9-(2′-(naphthalen-2-yl)-[1,1′-biphenyl]-3-yl)-9H-carbazole as a white solid (yield: 80%).

The compound was identified by FDMS measurement.

FDMS: 479

### Synthesis Example 36 (Synthesis of 4-chloro-9-(4-phenylnaphthalen-1-yl)-9H-carbazole)

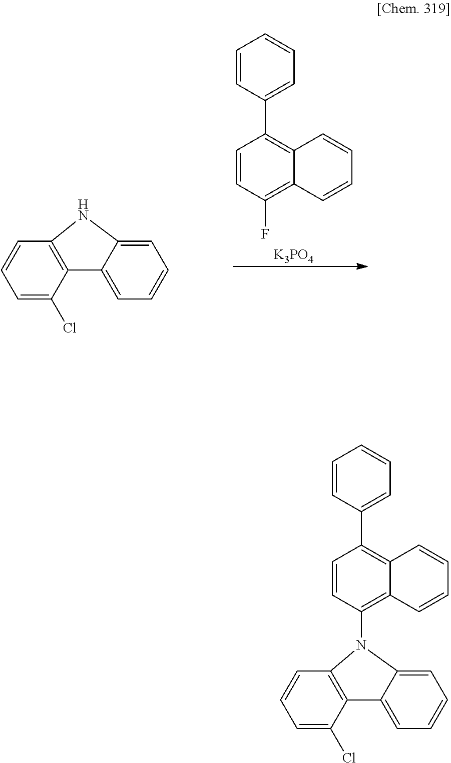

6.0 g (30 mmol) of 2-chloro-9H-carbazole, 7.9 g (36 mmol) of 1-fluoro-4-phenylnaphthalene, 13 g (60 mmol) of tripotassium phosphate and 70 mL of dimethyl sulfoxide were added to a 300-mL three-neck flask and were stirred at 180° C. for 15 hours. After cooling to room temperature, 150 mL of pure water was added and stirred to precipitate a solid. The solid was then filtered off and washed with water and hexane. The solid was recrystallized with a mixed solvent of toluene and butanol to isolate 10 g (25 mmol) of 4-chloro-9-(4-phenylnaphthalen-1-yl)-9H-carbazole as a white solid (yield: 83%).

The compound was identified by FDMS measurement.

FDMS: 403

### Synthesis Example 37 (Synthesis of 9-(4-([1,1′-biphenyl]-4-yl)naphthalen-1-yl)-4-chloro-9H-carbazole)

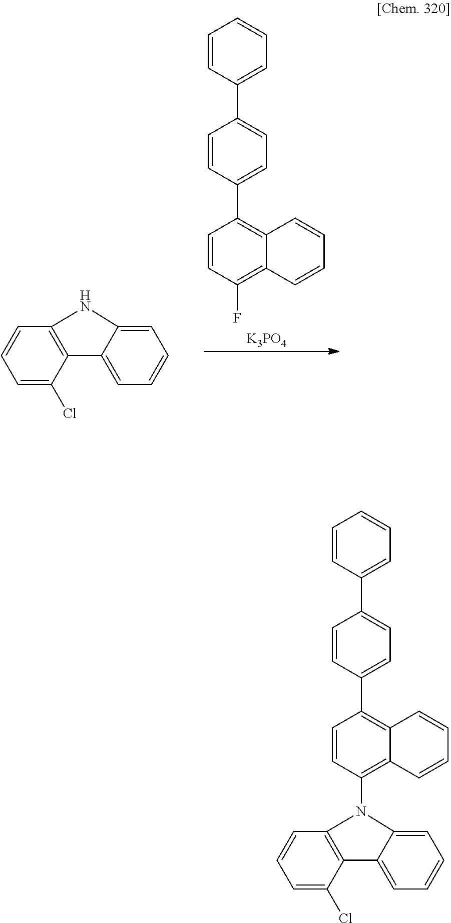

6.0 g (30 mmol) of 2-chloro-9H-carbazole, 11 g (36 mmol) of 1-([1,1′-biphenyl]-4-yl)-4-fluoronaphthalene, 13 g (60 mmol) of tripotassium phosphate and 100 mL of dimethyl sulfoxide were added to a 300-mL three-neck flask and were stirred at 180° C. for 15 hours. After cooling to room temperature, 150 m L of pure water was added and stirred to precipitate a solid. The solid was then filtered off and washed with water and hexane. The solid was recrystallized with a mixed solvent of toluene and butanol to isolate 12 g (26 mmol) of 9-(4-([1,1′-biphenyl]-4-yl)naphthalen-1-yl)-4-chloro-9H-carbazole as a white solid (yield: 87%).

The compound was identified by FDMS measurement.

FDMS: 479

### Synthesis Example 38 (Synthesis of 4-chloro-9-(2-phenylnaphthalen-1-yl)-9H-carbazole)

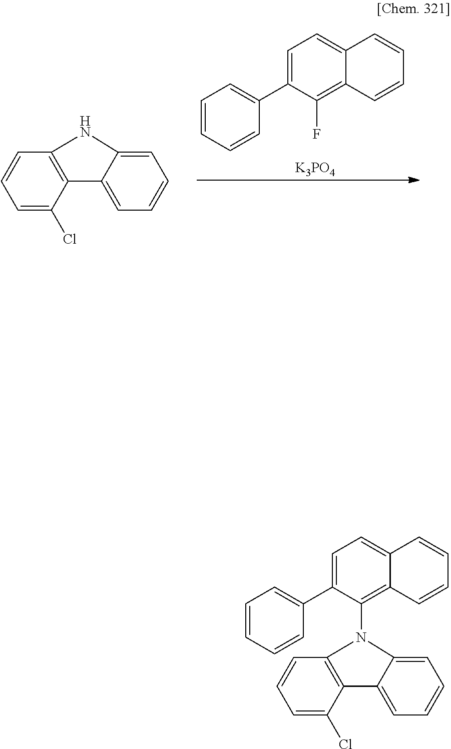

5.5 g (27 mmol) of 2-chloro-9H-carbazole, 7.3 g (33 mmol) of 1-fluoro-2-phenylnaphthalene, 12 g (55 mmol) of tripotassium phosphate and 60 mL of dimethyl sulfoxide were added to a 300-mL three-neck flask and were stirred at 180° C. for 15 hours. After cooling to room temperature, 150 mL of pure water was added and stirred to precipitate a solid. The solid was then filtered off and washed with water and hexane. The solid was recrystallized with a mixed solvent of toluene and butanol to isolate 8.5 g (21 mmol) of 4-chloro-9-(2-phenylnaphthalen-1-yl)-9H-carbazole as a white solid (yield: 77%).

The compound was identified by FDMS measurement.

FDMS: 403

### Synthesis Example 39 (Synthesis of 2-chloro-9-(4,4″-dimethyl-[1,1′:3′,1″-terphenyl]-4′-yl)-9H-carbazole)

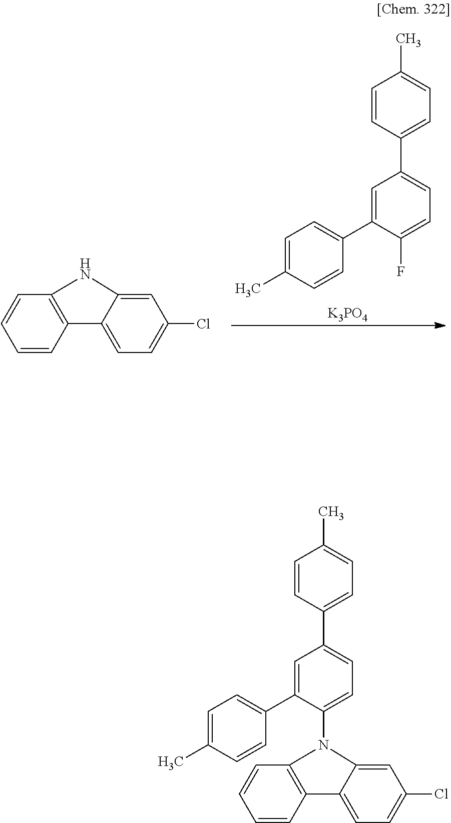

5.0 g (25 mmol) of 2-chloro-9H-carbazole, 8.2 g (30 mmol) of 4′-fluoro-4,4″-dimethyl-1,1′:3′,1″-terphenyl, 11 g (50 mmol) of tripotassium phosphate and 100 mL of dimethyl sulfoxide were added to a 300-mL three-neck flask and were stirred at 180° C. for 15 hours. After cooling to room temperature, 50 mL of pure water was added and stirred to precipitate a solid. The solid was then filtered off and washed with water and hexane. The solid was recrystallized with a mixed solvent of toluene and butanol to isolate 7.9 g (17 mmol) of 2-chloro-9-(4,4″-dimethyl-[1,1′:3′,1″-terphenyl]-4′-yl)-9H-carbazole as a white solid (yield: 70%).

The compound was identified by FDMS measurement.

FDMS: 423

### Synthesis Example 40 (Synthesis of 4-chloro-9-(1-phenylnaphthalen-2-yl)-9H-carbazole)

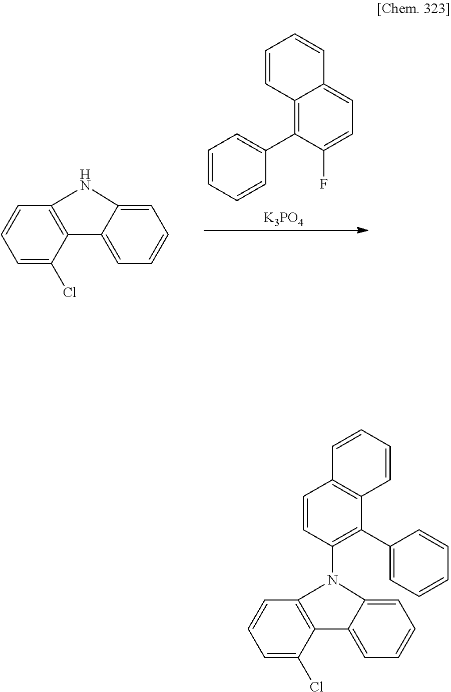

5.0 g (25 mmol) of 2-chloro-9H-carbazole, 6.6 g (30 mmol) of 2-fluoro-1-phenylnaphthalene, 11 g (50 mmol) of tripotassium phosphate and 50 mL of dimethyl sulfoxide were added to a 300-mL three-neck flask and were stirred at 180° C. for 15 hours. After cooling to room temperature, 150 mL of pure water was added and stirred to precipitate a solid. The solid was then filtered off and washed with water and hexane. The solid was recrystallized with a mixed solvent of toluene and butanol to isolate 8.1 g (20 mmol) of 4-chloro-9-(1-phenylnaphthalen-2-yl)-9H-carbazole as a white solid (yield: 81%).

The compound was identified by FDMS measurement.

FDMS: 403

### Synthesis Example 41 (Synthesis of 9-([1,1:3′,1″-terphenyl]-2-yl)-4-chloro-9H-carbazole)

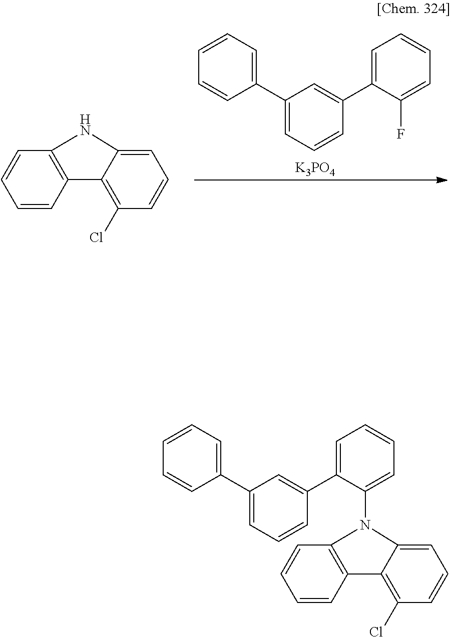

7.5 g (37 mmol) of 2-chloro-9H-carbazole, 11 g (45 mmol) of 2-fluoro-1,1′:3′,1″-terphenyl, 16 g (74 mmol) of tripotassium phosphate and 80 mL of dimethyl sulfoxide were added to a 300-mL three-neck flask and were stirred at 180° C. for 15 hours. After cooling to room temperature, 150 mL of pure water was added and stirred to precipitate a solid. The solid was then filtered off and washed with water and hexane. The solid was recrystallized with a mixed solvent of toluene and butanol to isolate 12 g (28 mmol) of 9-([1,1′:3′,1″-terphenyl]-2-yl)-4-chloro-9H-carbazole as a white solid (yield: 76%).

The compound was identified by FDMS measurement.

FDMS: 429

### Synthesis Example 42 (Synthesis of 4-chloro-9-(2-(dibenzo[b,d]thiophen-2-yl)-5-methylphenyl)-9H-carbazole)

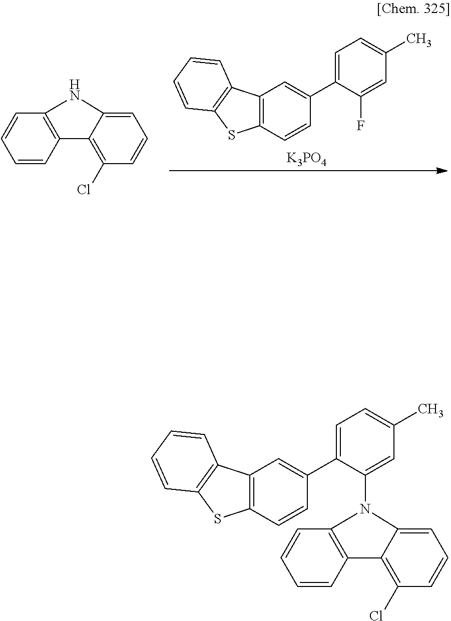

3.0 g (15 mmol) of 2-chloro-9H-carbazole, 5.2 g (18 mmol) of 2-(2-fluoro-4-methylphenyl)dibenzo[b,d]thiophene, 6.3 g (30 mmol) of tripotassium phosphate and 100 mL of dimethyl sulfoxide were added to a 300-mL three-neck flask and were stirred at 180° C. for 15 hours. After cooling to room temperature, 150 mL of pure water was added and stirred to precipitate a solid. The solid was then filtered off and washed with water and hexane. The solid was recrystallized with a mixed solvent of toluene and butanol to isolate 4.4 g (9.4 mmol) of 4-chloro-9-(2-(dibenzo[b,d]thiophen-2-yl)-5-methylphenyl)-9H-carbazole as a white solid (yield: 63%).

The compound was identified by FDMS measurement.

FDMS: 473

### Example 1 (Synthesis of Compound (D68))

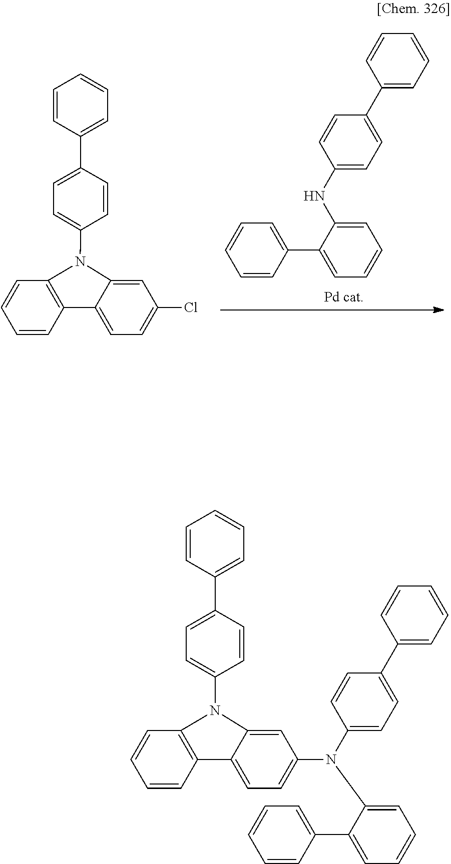 [SMILES: Clc1ccc2c3ccccc3n(-c3ccc(-c4ccccc4)cc3)c2c1 ; c1ccc(-c2ccc(Nc3ccccc3-c3ccccc3)cc2)cc1 ; c1ccc(-c2ccc(N(c3ccc4c5ccccc5n(-c5ccc(-c6ccccc6)cc5)c4c3)c3ccccc3-c3ccccc3)cc2)cc1]

In a nitrogen stream, 3.1 g (8.7 mmol) of 9-([1,1′-biphenyl]-4-yl)-2-chloro-9H-carbazole prepared in Synthesis Example 1, 2.8 g (8.7 mmol) of N-([1,1′-biphenyl]-4-yl)-[1,1′-biphenyl]-2-amine, 1.0 g (10 mmol) of sodium-tert-butoxide, 20 mL of xylene, 20 mg (87 μmol) of palladium acetate and 0.21 g (0.26 mmol) of a 25% by weight xylene solution of tri(tert-butyl)phosphine were added to a 100-mL three-neck flask and were stirred at 140° C. for 22 hours. After cooling to room temperature, 22 mL of pure water was added thereto and stirred. An aqueous layer and an organic layer were then separated, and the organic layer was washed with saturated aqueous sodium chloride. The organic layer was dried over anhydrous magnesium sulfate and was then subjected to column chromatography with a small amount of silica gel to remove a polar component. The solvent was then distilled off under reduced pressure, and the resulting solid was recrystallized with a mixed solvent of toluene and butanol to isolate 4.0 g (6.3 mmol) of a compound (D68) as a white solid (yield: 72%). D68 had a sublimation temperature of 300° C., and it was confirmed that the sublimate D68 was glassy.

The compound was identified by FDMS measurement.

FDMS: 638

### Example 2 (Synthesis of D116)

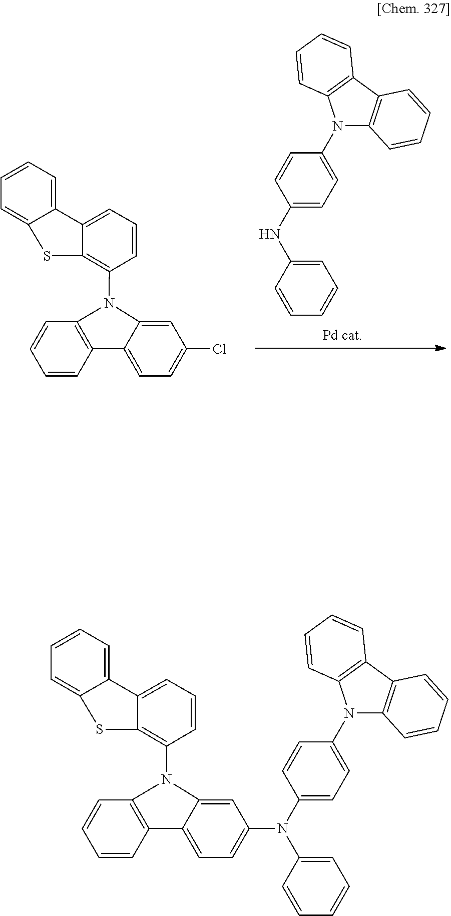 [SMILES: Clc1ccc2c3ccccc3n(-c3cccc4c3sc3ccccc34)c2c1 ; c1ccc(Nc2ccc(-n3c4ccccc4c4ccccc43)cc2)cc1 ; c1ccc(N(c2ccc(-n3c4ccccc4c4ccccc43)cc2)c2ccc3c4ccccc4n(-c4cccc5c4sc4ccccc45)c3c2)cc1]

In a nitrogen stream, 3.8 g (9.9 mmol) of 2-chloro-9-(dibenzo[b,d]thiophen-4-yl)-9H-carbazole prepared in Synthesis Example 9, 3.0 g (9.0 mmol) of 4-(9H-carbazol-9-yl)-N-phenylaniline, 1.0 g (11 mmol) of sodium-tert-butoxide, 30 mL of xylene, 20 mg (90 μmol) of palladium acetate and 0.22 g (0.27 mmol) of a 25% by weight xylene solution of tri(tert-butyl)phosphine were added to a 100-mL three-neck flask and were stirred at 140° C. for 16 hours. After cooling to room temperature, 22 mL of pure water was added thereto and stirred. An aqueous layer and an organic layer were then separated, and the organic layer was washed with saturated aqueous sodium chloride. The organic layer was dried over anhydrous magnesium sulfate and was then subjected to column chromatography with a small amount of silica gel to remove a polar component. The solvent was then distilled off under reduced pressure, and the resulting solid was recrystallized with a mixed solvent of toluene and butanol to isolate 4.2 g (6.2 mmol) of a compound (D116) as a white solid (yield: 69%). D116 had a sublimation temperature of 310° C., and it was confirmed that the sublimate D116 was glassy.

The compound was identified by FDMS measurement.

FDMS: 681

### Example 3 (Synthesis of Compound (D166))

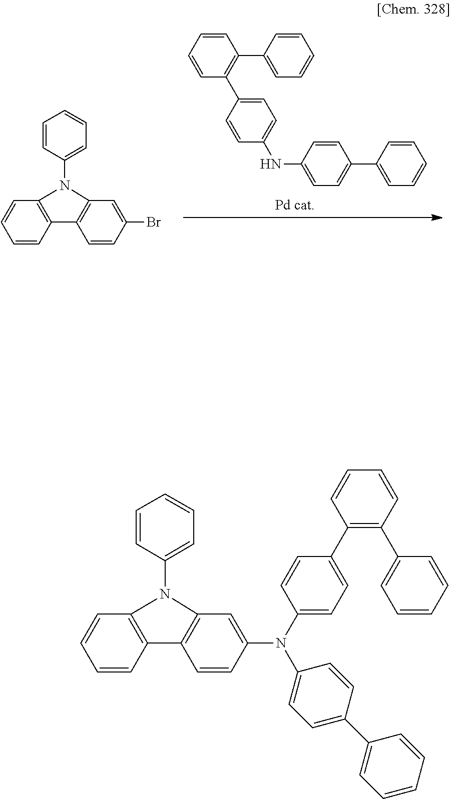 [SMILES: Brc1ccc2c3ccccc3n(-c3ccccc3)c2c1 ; c1ccc(-c2ccc(Nc3ccc(-c4ccccc4-c4ccccc4)cc3)cc2)cc1 ; c1ccc(-c2ccc(N(c3ccc(-c4ccccc4-c4ccccc4)cc3)c3ccc4c5ccccc5n(-c5ccccc5)c4c3)cc2)cc1]

In a nitrogen stream, 2.5 g (7.8 mmol) of 9-phenyl-2-bromocarbazole, 2.6 g (6.5 mmol) of N-(p-biphenyl-4-yl)-N-(o-terphenyl-4-yl)amine prepared in Synthesis Example 20, 1.3 g (0.82 mmol) of sodium-tert-butoxide, 22 mL of o-xylene, 4.4 mg (20 μmol) of palladium acetate and 48 mg (59 μmol) of a 25% by weight toluene solution of tri(tert-butyl)phosphine were added to a 100-mL three-neck flask and were stirred at 140° C. for 20 hours. After cooling to room temperature, 22 mL of pure water was added thereto and stirred. An aqueous layer and an organic layer were then separated, and the organic layer was washed with saturated aqueous sodium chloride. The organic layer was dried over anhydrous magnesium sulfate and was then subjected to column chromatography with a small amount of silica gel to remove a polar component. The solvent was then distilled off under reduced pressure, and the resulting solid was recrystallized with a mixed solvent of toluene and butanol to isolate 2.5 g (4.4 mmol) of a compound (D165) as a white solid (yield: 68%). D166 had a sublimation temperature of 310° C., and it was confirmed that the sublimate D166 was glassy.

The compound was identified by FDMS measurement.

FDMS: 612

### Example 4 (Synthesis of Compound (D169))

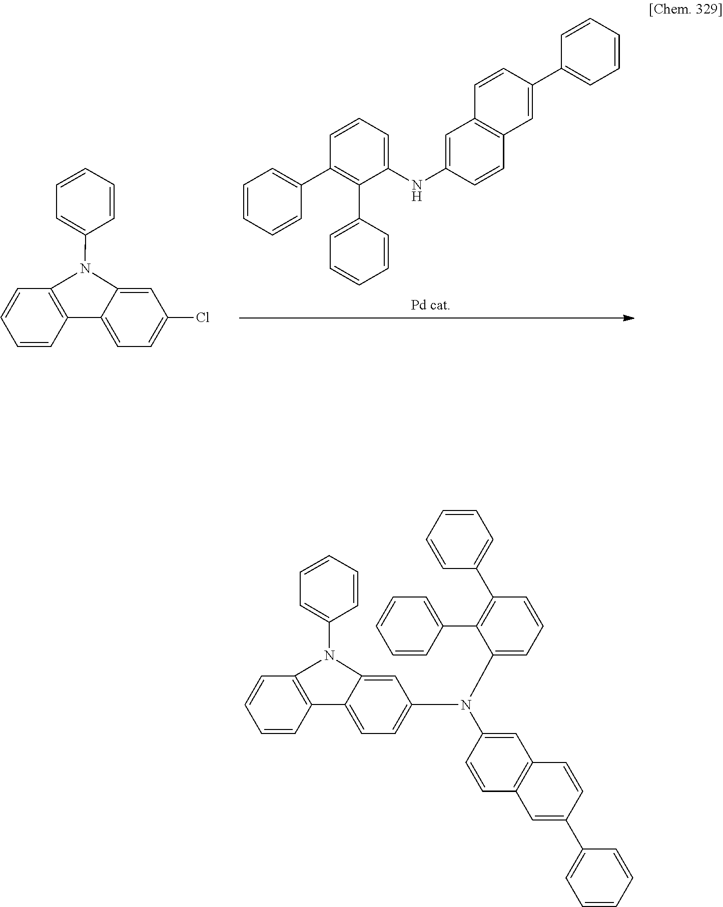 [SMILES: Clc1ccc2c3ccccc3n(-c3ccccc3)c2c1 ; c1ccc(-c2ccc3cc(Nc4cccc(-c5ccccc5)c4-c4ccccc4)ccc3c2)cc1 ; c1ccc(-c2ccc3cc(N(c4ccc5c6ccccc6n(-c6ccccc6)c5c4)c4cccc(-c5ccccc5)c4-c4ccccc4)ccc3c2)cc1]

In a nitrogen stream, 1.8 g (6.3 mmol) of 2-chloro-9-phenyl-9H-carbazole, 2.7 g (6.0 mmol) of N-([1,1′:2′,1″-terphenyl]-3′-yl)-6-phenylnaphthalene-2-amine, 0.75 g (7.8 mmol) of sodium-tert-butoxide, 20 mL of xylene, 14 mg (60 μmol) of palladium acetate and 0.15 g (0.18 mmol) of a 25% by weight xylene solution of tri(tert-butyl)phosphine were added to a 100-mL three-neck flask and were stirred at 140° C. for 22 hours. After cooling to room temperature, 22 mL of pure water was added thereto and stirred. An aqueous layer and an organic layer were then separated, and the organic layer was washed with saturated aqueous sodium chloride. The organic layer was dried over anhydrous magnesium sulfate and was then subjected to column chromatography with a small amount of silica gel to remove a polar component. The solvent was then distilled off under reduced pressure, and the resulting solid was recrystallized with a mixed solvent of toluene and butanol to isolate 3.1 g (4.5 mmol) of N-([1,1′:2′,1″-terphenyl]-3′-yl)-9-phenyl-N-(6-phenylnaphthalen-2-yl)-9H-carbazole-2-amine as a white solid of a compound (D169) (yield: 74%). D169 had a sublimation temperature of 315° C., and it was confirmed that the sublimate D169 was glassy.

The compound was identified by FDMS measurement.

FDMS: 688

### Example 5 (Synthesis of Compound (D180))

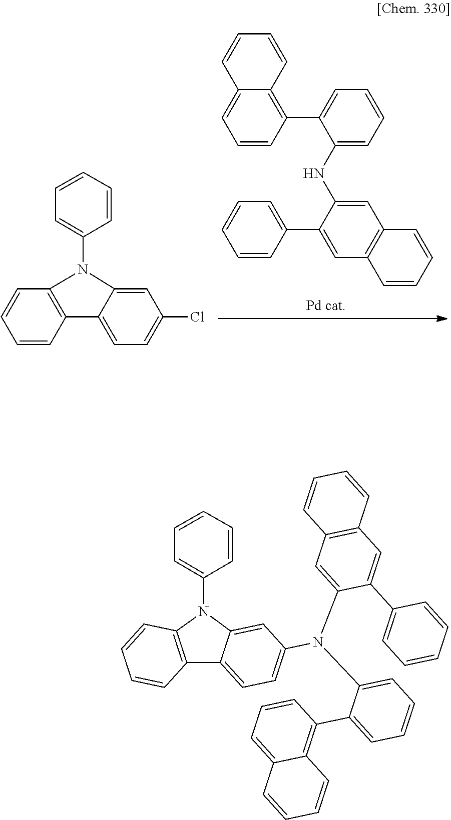 [SMILES: Clc1ccc2c3ccccc3n(-c3ccccc3)c2c1 ; c1ccc(-c2cc3ccccc3cc2Nc2ccccc2-c2cccc3ccccc23)cc1 ; c1ccc(-c2cc3ccccc3cc2N(c2ccc3c4ccccc4n(-c4ccccc4)c3c2)c2ccccc2-c2cccc3ccccc23)cc1]

In a nitrogen stream, 2.1 g (7.7 mmol) of 2-chloro-9-phenyl-9H-carbazole, 3.0 g (7.3 mmol) of N-(2-(dibenzo[b,d]furan-4-yl)phenyl)-[1,1′-biphenyl]-4-amine, 0.91 g (9.5 mmol) of sodium-tert-butoxide, 20 mL of xylene, 16 mg (73 μmol) of palladium acetate and 0.18 g (0.22 mmol) of a 25% by weight xylene solution of tri(tert-butyl)phosphine were added to a 100-mL three-neck flask and were stirred at 140° C. for 22 hours. After cooling to room temperature, 22 mL of pure water was added thereto and stirred. An aqueous layer and an organic layer were then separated, and the organic layer was washed with saturated aqueous sodium chloride. The organic layer was dried over anhydrous magnesium sulfate and was then subjected to column chromatography with a small amount of silica gel to remove a polar component. The solvent was then distilled off under reduced pressure, and the resulting solid was recrystallized with a mixed solvent of toluene and butanol to isolate 3.6 g (5.5 mmol) of a compound (D180) as a white solid (yield: 76%). D180 had a sublimation temperature of 270° C., and it was confirmed that the sublimate D180 was glassy.

The compound was identified by FDMS measurement.

FDMS: 652

### Example 6 (Synthesis of Compound (D184))

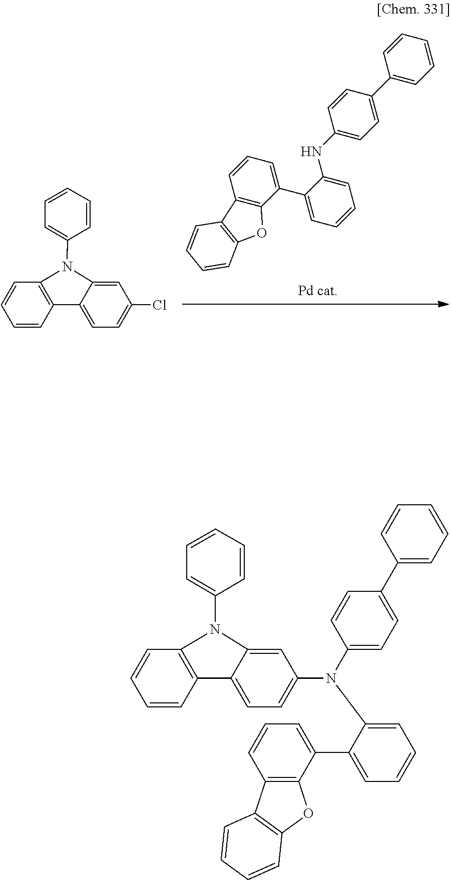 [SMILES: Clc1ccc2c3ccccc3n(-c3ccccc3)c2c1 ; c1ccc(-c2ccc(Nc3ccccc3-c3cccc4c3oc3ccccc34)cc2)cc1 ; c1ccc(-c2ccc(N(c3ccc4c5ccccc5n(-c5ccccc5)c4c3)c3ccccc3-c3cccc4c3oc3ccccc34)cc2)cc1]

In a nitrogen stream, 2.7 g (6.2 mmol) of 9-([1,1′:3′,1″-terphenyl]-5′-yl)-2-chloro-9H-carbazole, 2.5 g (5.9 mmol) of N-([1,1′:4′,1″-terphenyl]-4-yl)phenanthrene-9-amine, 0.74 g (7.7 mmol) of sodium-tert-butoxide, 20 mL of xylene, 13 mg (59 μmol) of palladium acetate and 0.14 g (0.18 mmol) of a 25% by weight xylene solution of tri(tert-butyl)phosphine were added to a 100-mL three-neck flask and were stirred at 140° C. for 22 hours. After cooling to room temperature, 22 mL of pure water was added thereto and stirred. An aqueous layer and an organic layer were then separated, and the organic layer was washed with saturated aqueous sodium chloride. The organic layer was dried over anhydrous magnesium sulfate and was then subjected to column chromatography with a small amount of silica gel to remove a polar component. The solvent was then distilled off under reduced pressure, and the resulting solid was recrystallized with a mixed solvent of toluene and butanol to isolate 3.5 g (4.3 mmol) of a compound (D184) as a white solid (yield: 73%). D184 had a sublimation temperature of 285° C., and it was confirmed that the sublimate D184 was glassy.

The compound was identified by FDMS measurement.

FDMS: 423

### Example 7 (Synthesis of Compound (D188))

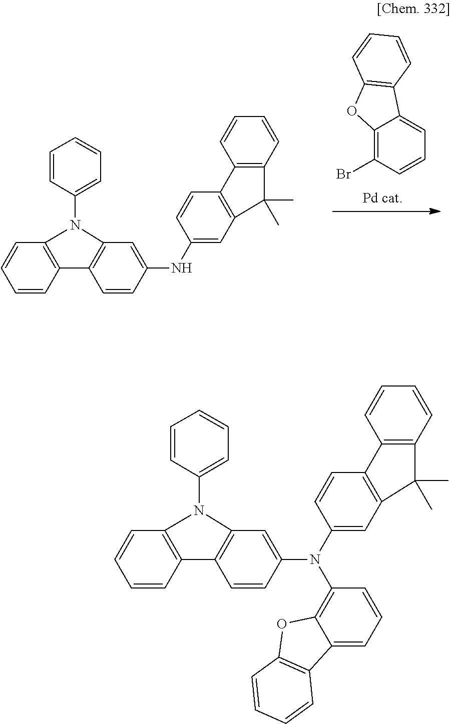 [SMILES: CC1(C)c2ccccc2-c2ccc(Nc3ccc4c5ccccc5n(-c5ccccc5)c4c3)cc21 ; Brc1cccc2c1oc1ccccc12 ; CC1(C)c2ccccc2-c2ccc(N(c3ccc4c5ccccc5n(-c5ccccc5)c4c3)c3cccc4c3oc3ccccc34)cc21]

In a nitrogen stream, 2.4 g (7.4 mmol) of N-(9,9-dimethylfluoren-2-yl)-N-(9-phenylcarbazol-2-yl)amine, 2.3 g (6.1 mmol) of 4-bromodibenzofuran, 0.77 g (8.0 mmol) of sodium-tert-butoxide, 20 mL of o-xylene, 4.1 mg (18 μmol) of palladium acetate and 45 mg (55 μmol) of a 25% by weight toluene solution of tri(tert-butyl)phosphine were added to a 100-mL three-neck flask and were stirred at 140° C. for 20 hours. After cooling to room temperature, 20 mL of pure water was added thereto and stirred. An aqueous layer and an organic layer were then separated, and the organic layer was washed with saturated aqueous sodium chloride. The organic layer was dried over anhydrous magnesium sulfate and was then subjected to column chromatography with a small amount of silica gel to remove a polar component. The solvent was then distilled off under reduced pressure, and the resulting solid was recrystallized with a mixed solvent of toluene and butanol to isolate 3.0 g (4.0 mmol) of a compound (D188) as a yellow solid (yield: 80%). D188 had a sublimation temperature of 325° C., and it was confirmed that the sublimate D188 was glassy.

The compound was identified by FDMS measurement.

FDMS: 616

### Example 8 (Synthesis of Compound (D194))

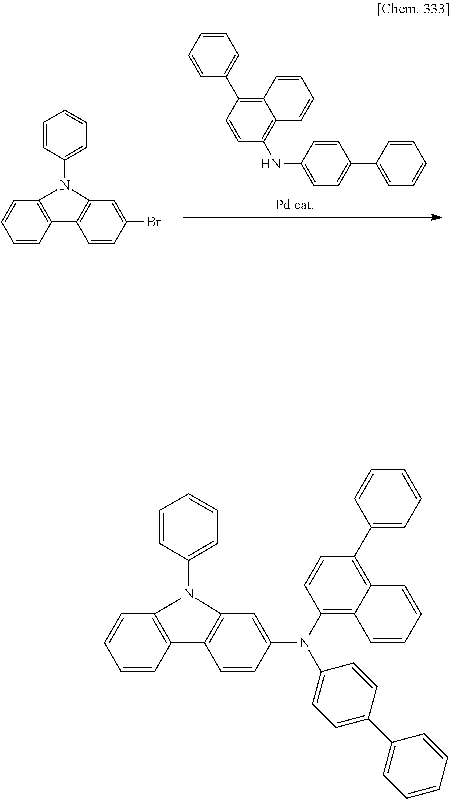 [SMILES: Brc1ccc2c3ccccc3n(-c3ccccc3)c2c1 ; c1ccc(-c2ccc(Nc3ccc(-c4ccccc4)c4ccccc34)cc2)cc1 ; c1ccc(-c2ccc(N(c3ccc4c5ccccc5n(-c5ccccc5)c4c3)c3ccc(-c4ccccc4)c4ccccc34)cc2)cc1]

In a nitrogen stream, 3.4 g (11 mmol) of 9-phenyl-2-bromocarbazole, 3.3 g (8.9 mmol) of N-(biphenyl-4-yl)-N-(4-phenylnaphthalen-1-yl)amine, 1.1 g (12 mmol) of sodium-tert-butoxide, 30 mL of o-xylene, 6.0 mg (27 μmol) of palladium acetate and 65 mg (80 μmol) of a 25% by weight toluene solution of tri(tert-butyl)phosphine were added to a 100-mL three-neck flask and were stirred at 140° C. for 22 hours. After cooling to room temperature, 37 mL of pure water was added thereto and stirred. An aqueous layer and an organic layer were then separated, and the organic layer was washed with saturated aqueous sodium chloride. The organic layer was dried over anhydrous magnesium sulfate and was then subjected to column chromatography with a small amount of silica gel to remove a polar component. The solvent was then distilled off under reduced pressure, and the resulting solid was recrystallized with a mixed solvent of toluene and butanol to isolate 4.8 g (7.9 mmol) of a compound (D194) as a white solid (yield: 89%). D194 had a sublimation temperature of 300° C., and it was confirmed that the sublimate D194 was glassy.

The compound was identified by FDMS measurement.

FDMS: 612

### Example 9 (Synthesis of Compound (D273))

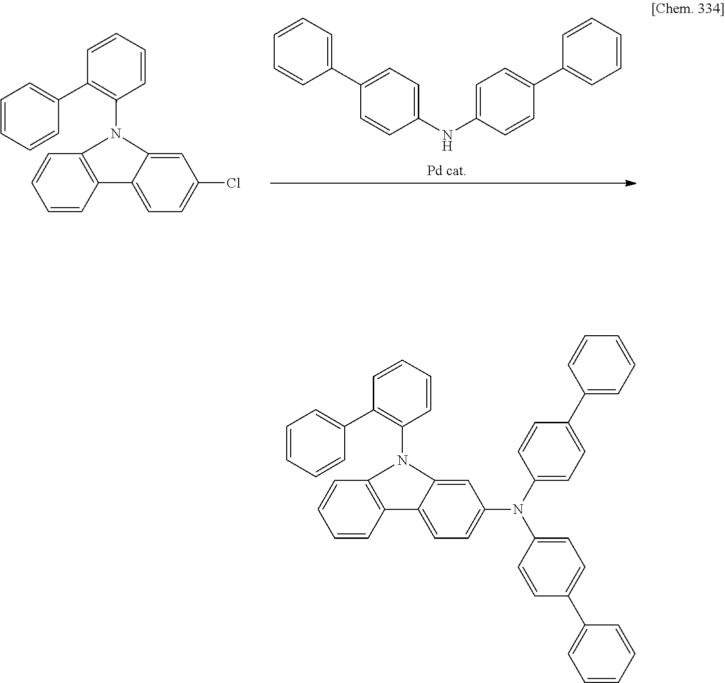 [SMILES: Clc1ccc2c3ccccc3n(-c3ccccc3-c3ccccc3)c2c1 ; c1ccc(-c2ccc(Nc3ccc(-c4ccccc4)cc3)cc2)cc1 ; c1ccc(-c2ccc(N(c3ccc(-c4ccccc4)cc3)c3ccc4c5ccccc5n(-c5ccccc5-c5ccccc5)c4c3)cc2)cc1]

In a nitrogen stream, 2.2 g (6.2 mmol) of 9-([1,1′-biphenyl]-2-yl)-2-chloro-9H-carbazole prepared in Synthesis Example 2, 1.9 g (5.9 mmol) of N-([1,1′:4′,1″-terphenyl]-4-yl)phenanthrene-9-amine, 0.74 g (7.7 mmol) of sodium-tert-butoxide, 20 mL of xylene, 13 mg (59 μmol) of palladium acetate and 0.14 g (0.18 mmol) of a 25% by weight xylene solution of tri(tert-butyl)phosphine were added to a 100-mL three-neck flask and were stirred at 140° C. for 8 hours. After cooling to room temperature, 40 mL of pure water was added thereto and stirred. An aqueous layer and an organic layer were then separated, and the organic layer was washed with saturated aqueous sodium chloride. The organic layer was dried over anhydrous magnesium sulfate and was then subjected to column chromatography with a small amount of silica gel to remove a polar component. The solvent was then distilled off under reduced pressure, and the resulting solid was recrystallized with a mixed solvent of toluene and butanol to isolate 3.2 g (5.0 mmol) of a compound (D273) as a white solid (yield: 84%). D273 had a sublimation temperature of 270° C., and it was confirmed that the sublimate D273 was glassy.

The compound was identified by FDMS measurement.

FDMS: 638

### Example 10 (Synthesis of Compound (D278))

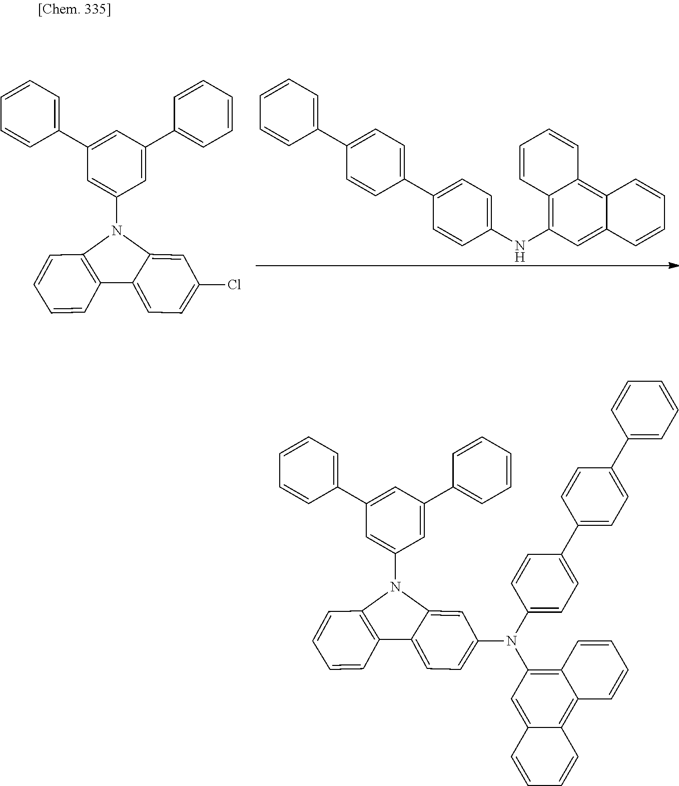

In a nitrogen stream, 2.7 g (6.2 mmol) of 9-([1,1′:3′,1″-terphenyl]-5′-yl)-2-chloro-9H-carbazole prepared in Synthesis Example 7, 2.5 g (5.9 mmol) of N-([1,1′:4′,1″-terphenyl]-4-yl)phenanthrene-9-amine, 0.74 g (7.7 mmol) of sodium-tert-butoxide, 20 mL of xylene, 13 mg (59 μmol) of palladium acetate and 0.14 g (0.18 mmol) of a 25% by weight xylene solution of tri(tert-butyl)phosphine were added to a 100-mL three-neck flask and were stirred at 140° C. for 22 hours. After cooling to room temperature, 22 mL of pure water was added thereto and stirred. An aqueous layer and an organic layer were then separated, and the organic layer was washed with saturated aqueous sodium chloride. The organic layer was dried over anhydrous magnesium sulfate and was then subjected to column chromatography with a small amount of silica gel to remove a polar component. The solvent was then distilled off under reduced pressure, and the resulting solid was recrystallized with a mixed solvent of toluene and butanol to isolate 3.5 g (4.3 mmol) of a compound (D278) as a white solid (yield: 73%). D278 had a sublimation temperature of 330° C., and it was confirmed that the sublimate D278 was glassy.

The compound was identified by FDMS measurement.

FDMS: 814

### Example 11 (Synthesis of Compound (D285))

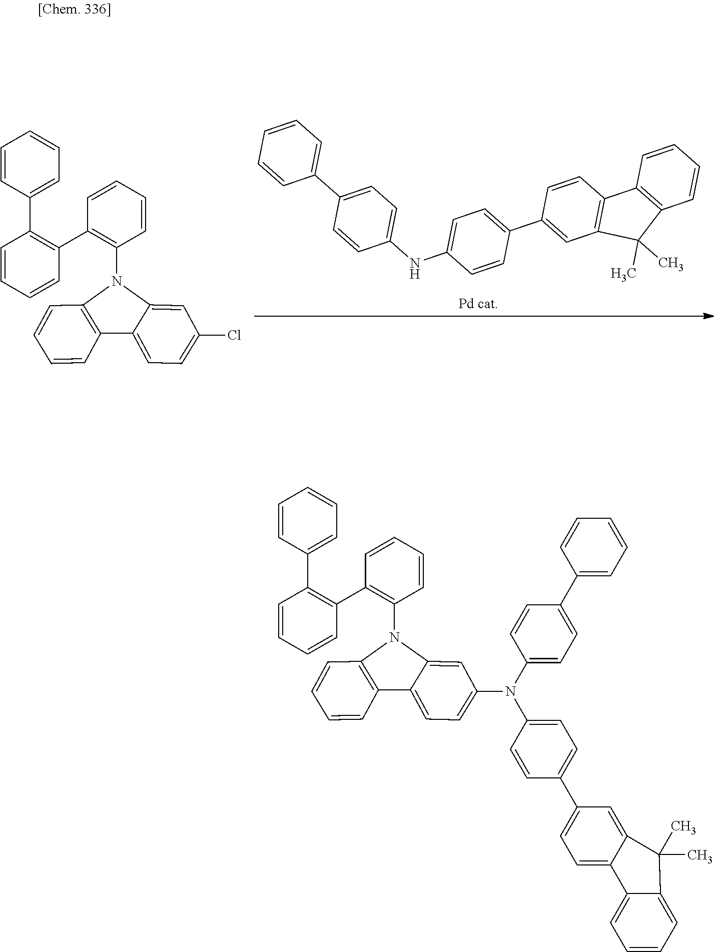 [SMILES: Clc1ccc2c3ccccc3n(-c3ccccc3-c3ccccc3-c3ccccc3)c2c1 ; CC1(C)c2ccccc2-c2ccc(-c3ccc(Nc4ccc(-c5ccccc5)cc4)cc3)cc21 ; CC1(C)c2ccccc2-c2ccc(-c3ccc(N(c4ccc(-c5ccccc5)cc4)c4ccc5c6ccccc6n(-c6ccccc6-c6ccccc6-c6ccccc6)c5c4)cc3)cc21]

In a nitrogen stream, 2.5 g (5.8 mmol) of 9-([1,1′:2′,1″-terphenyl]-2-yl)-2-chloro-9H-carbazole prepared in Synthesis Example 10, 2.4 g (5.5 mmol) of N-(4-(9,9-dimethyl-9H-fluoren-2-yl)phenyl)-[1,1′-biphenyl]-4-amine, 0.69 g (7.1 mmol) of sodium-tert-butoxide, 20 mL of xylene, 12 mg (55 μmol) of palladium acetate and 0.13 g (0.16 mmol) of a 25% by weight xylene solution of tri(tert-butyl)phosphine were added to a 100-mL three-neck flask and were stirred at 140° C. for 22 hours. After cooling to room temperature, 22 mL of pure water was added thereto and stirred. An aqueous layer and an organic layer were then separated, and the organic layer was washed with saturated aqueous sodium chloride. The organic layer was dried over anhydrous magnesium sulfate and was then subjected to column chromatography with a small amount of silica gel to remove a polar component. The solvent was then distilled off under reduced pressure, and the resulting solid was recrystallized with a mixed solvent of toluene and butanol to isolate 3.2 g (3.8 mmol) of a compound (D285) as a white solid (yield: 70%). D285 had a sublimation temperature of 300° C., and it was confirmed that the sublimate D285 was glassy.

The compound was identified by FDMS measurement.

FDMS: 423

### Example 12 (Synthesis of Compound (D288))

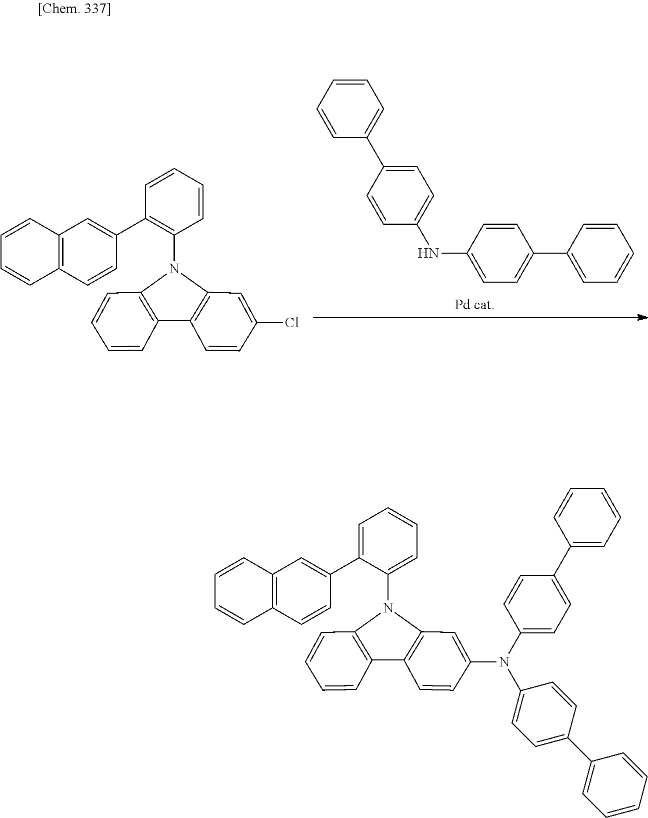 [SMILES: Clc1ccc2c3ccccc3n(-c3ccccc3-c3ccc4ccccc4c3)c2c1 ; c1ccc(-c2ccc(Nc3ccc(-c4ccccc4)cc3)cc2)cc1 ; c1ccc(-c2ccc(N(c3ccc(-c4ccccc4)cc3)c3ccc4c5ccccc5n(-c5ccccc5-c5ccc6ccccc6c5)c4c3)cc2)cc1]

In a nitrogen stream, 2.9 g (7.1 mmol) of 9-(2-(naphthalen-2-yl)phenyl)-2-chlorocarbazole prepared in Synthesis Example 6, 1.9 g (5.9 mmol) of N,N-bis(biphenyl-4-yl)amine, 0.74 g (7.7 mmol) of sodium-tert-butoxide, 20 mL of o-xylene, 4.0 mg (18 μmol) of palladium acetate and 43 mg (53 μmol) of a 25% by weight toluene solution of tri(tert-butyl)phosphine were added to a 100-mL three-neck flask and were stirred at 140° C. for 22 hours. After cooling to room temperature, 20 mL of pure water was added thereto and stirred. An aqueous layer and an organic layer were then separated, and the organic layer was washed with saturated aqueous sodium chloride. The organic layer was dried over anhydrous magnesium sulfate and was then subjected to column chromatography with a small amount of silica gel to remove a polar component. The solvent was then distilled off under reduced pressure, and the resulting solid was recrystallized with a mixed solvent of toluene and butanol to isolate 2.8 g (4.0 mol) of a compound (D288) as a white solid (yield: 68%). D288 had a sublimation temperature of 340° C., and it was confirmed that the sublimate D288 was glassy.

The compound was identified by FDMS measurement.

FDMS: 688

### Example 13 (Synthesis of Compound (D289))

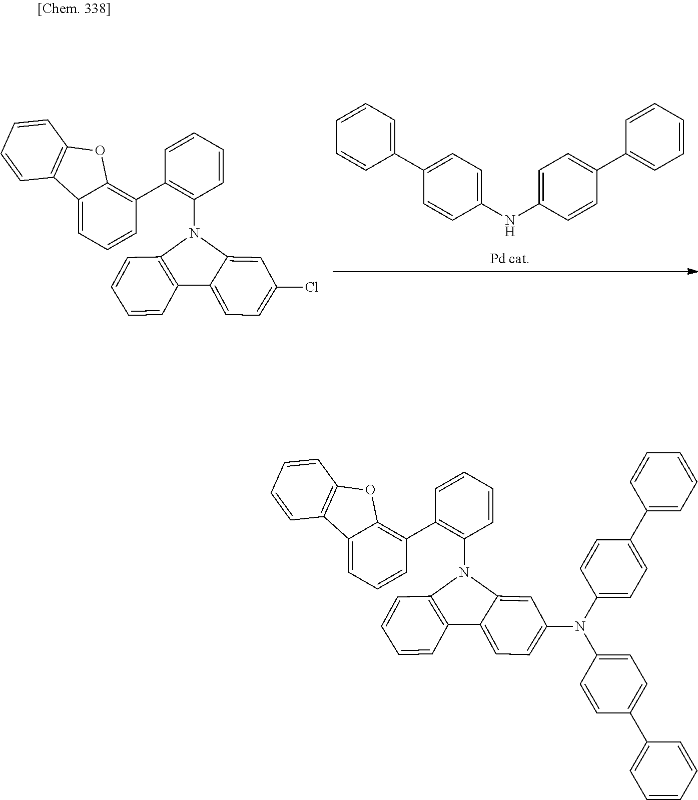 [SMILES: Clc1ccc2c3ccccc3n(-c3ccccc3-c3cccc4c3oc3ccccc34)c2c1 ; c1ccc(-c2ccc(Nc3ccc(-c4ccccc4)cc3)cc2)cc1 ; c1ccc(-c2ccc(N(c3ccc(-c4ccccc4)cc3)c3ccc4c5ccccc5n(-c5ccccc5-c5cccc6c5oc5ccccc56)c4c3)cc2)cc1]

In a nitrogen stream, 3.8 g (8.5 mmol) of 2-chloro-9-(2-(dibenzo[b,d]furan-4-yl)phenyl)-9H-carbazole prepared in Synthesis Example 11, 2.6 g (8.1 mmol) of di([1,1′-biphenyl]-4-yl)amine, 1.0 g (11 mmol) of sodium-tert-butoxide, 20 mL of xylene, 18 mg (81 μmol) of palladium acetate and 0.20 g (0.24 mmol) of a 25% by weight xylene solution of tri(tert-butyl)phosphine were added to a 100-mL three-neck flask and were stirred at 140° C. for 22 hours. After cooling to room temperature, 22 mL of pure water was added thereto and stirred. An aqueous layer and an organic layer were then separated, and the organic layer was washed with saturated aqueous sodium chloride. The organic layer was dried over anhydrous magnesium sulfate and was then subjected to column chromatography with a small amount of silica gel to remove a polar component. The solvent was then distilled off under reduced pressure, and the resulting solid was recrystallized with a mixed solvent of toluene and butanol to isolate 4.4 g (6.1 mmol) of a compound (D289) as a white solid (yield: 75%). D289 had a sublimation temperature of 280° C., and it was confirmed that the sublimate D289 was glassy.

The compound was identified by FDMS measurement.

FDMS: 728

### Example 14 (Synthesis of Compound (D291))

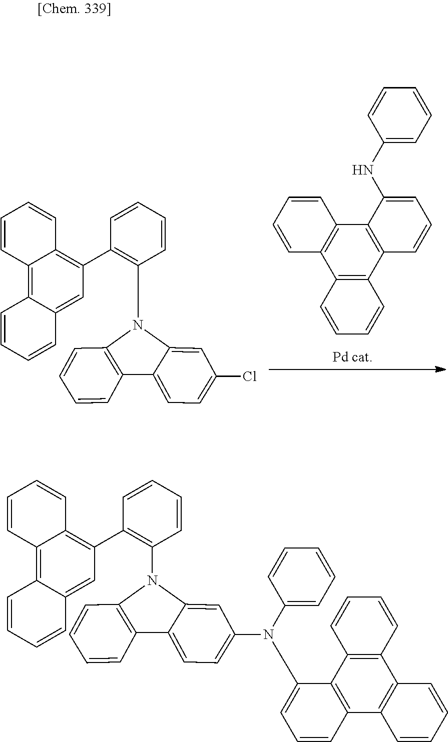 [SMILES: Clc1ccc2c3ccccc3n(-c3ccccc3-c3cc4ccccc4c4ccccc34)c2c1 ; c1ccc(Nc2cccc3c4ccccc4c4ccccc4c23)cc1 ; c1ccc(N(c2ccc3c4ccccc4n(-c4ccccc4-c4cc5ccccc5c5ccccc45)c3c2)c2cccc3c4ccccc4c4ccccc4c23)cc1]

In a nitrogen stream, 3.9 g (8.5 mmol) of 2-chloro-9-(2-(phenanthren-9-yl)phenyl)-9H-carbazole prepared in Synthesis Example 12, 2.6 g (8.1 mmol) of N-phenyltriphenylen-1-amine, 1.0 g (11 mmol) of sodium-tert-butoxide, 20 mL of xylene, 18 mg (81 μmol) of palladium acetate and 0.20 g (0.24 mmol) of a 25% by weight xylene solution of tri(tert-butyl)phosphine were added to a 100-mL three-neck flask and were stirred at 140° C. for 22 hours. After cooling to room temperature, 22 mL of pure water was added thereto and stirred. An aqueous layer and an organic layer were then separated, and the organic layer was washed with saturated aqueous sodium chloride. The organic layer was dried over anhydrous magnesium sulfate and was then subjected to column chromatography with a small amount of silica gel to remove a polar component. The solvent was then distilled off under reduced pressure, and the resulting solid was recrystallized with a mixed solvent of toluene and butanol to isolate 4.6 g (6.2 mmol) of a compound (D291) as a white solid (yield: 76%). D291 had a sublimation temperature of 300° C., and it was confirmed that the sublimate D291 was glassy.

The compound was identified by FDMS measurement.

FDMS: 736

### Example 15 (Synthesis of Compound (D312))

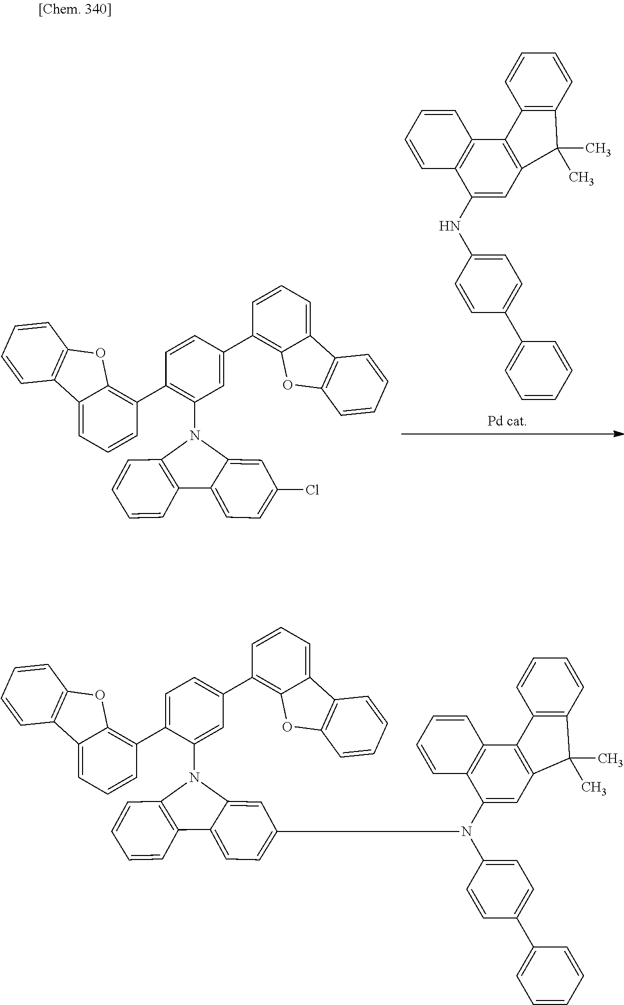 [SMILES: Clc1ccc2c3ccccc3n(-c3cc(-c4cccc5c4oc4ccccc45)ccc3-c3cccc4c3oc3ccccc34)c2c1 ; CC1(C)c2ccccc2-c2c1cc(Nc1ccc(-c3ccccc3)cc1)c1ccccc21 ; CC1(C)c2ccccc2-c2c1cc(N(c1ccc(-c3ccccc3)cc1)c1ccc3c4ccccc4n(-c4cc(-c5cccc6c5oc5ccccc56)ccc4-c4cccc5c4oc4ccccc45)c3c1)c1ccccc21]

In a nitrogen stream, 9-(2,5-bis(dibenzo[b,d]furan-4-yl)phenyl)-2-chloro-9H-carbazole (1.05 Eq) prepared in Synthesis Example 14, N-([1,1′-biphenyl]-4-yl)-7,7-dimethyl-7H-benzo[c]fluorene-5-amine (1 Eq), sodium-tert-butoxide (1.3 Eq), 20 mL of xylene, palladium acetate (0.01 Eq) and a 25% by weight xylene solution of tri(tert-butyl)phosphine (0.03 Eq) were added to a 100-mL three-neck flask and were stirred at 140° C. for 22 hours. After cooling to room temperature, 22 mL of pure water was added thereto and stirred. An aqueous layer and an organic layer were then separated, and the organic layer was washed with saturated aqueous sodium chloride. The organic layer was dried over anhydrous magnesium sulfate and was then subjected to column chromatography with a small amount of silica gel to remove a polar component. The solvent was then distilled off under reduced pressure, and the resulting solid was recrystallized with a mixed solvent of toluene and butanol to isolate a compound (D312) as a white solid (80%). D312 had a sublimation temperature of 340° C., and it was confirmed that the sublimate D312 was glassy.

The compound was identified by FDMS measurement.

FDMS: 984

### Example 16 (Synthesis of Compound (D336))

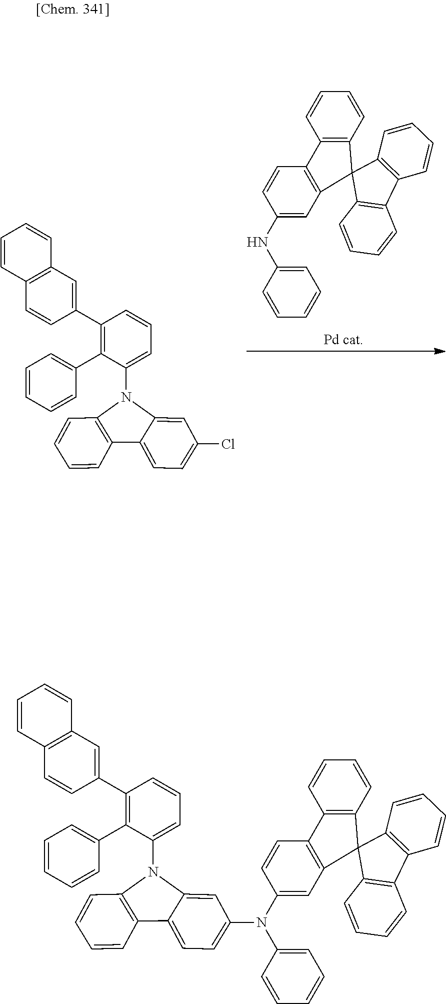 [SMILES: Clc1ccc2c3ccccc3n(-c3cccc(-c4ccc5ccccc5c4)c3-c3ccccc3)c2c1 ; c1ccc(Nc2ccc3c(c2)C2(c4ccccc4-c4ccccc42)c2ccccc2-3)cc1 ; c1ccc(-c2c(-c3ccc4ccccc4c3)cccc2-n2c3ccccc3c3ccc(N(c4ccccc4)c4ccc5c(c4)C4(c6ccccc6-c6ccccc64)c4ccccc4-5)cc32)cc1]

In a nitrogen stream, 3.1 g (6.4 mmol) of 2-chloro-9-(6-(naphthalen-2-yl)-[1,1′-biphenyl]-2-yl)-9H-carbazole prepared in Synthesis Example 16, 2.5 g (6.1 mmol) of N-phenyl-9,9′-spirobi[fluorene]-2-amine, 0.77 g (8.0 mmol) of sodium-tert-butoxide, 20 mL of xylene, 14 mg (61 μmol) of palladium acetate and 0.15 g (0.18 mmol) of a 25% by weight xylene solution of tri(tert-butyl)phosphine were added to a 100-mL three-neck flask and were stirred at 140° C. for 22 hours. After cooling to room temperature, 22 mL of pure water was added thereto and stirred. An aqueous layer and an organic layer were then separated, and the organic layer was washed with saturated aqueous sodium chloride. The organic layer was dried over anhydrous magnesium sulfate and was then subjected to column chromatography with a small amount of silica gel to remove a polar component. The solvent was then distilled off under reduced pressure, and the resulting solid was recrystallized with a mixed solvent of toluene and butanol to isolate 3.1 g (3.6 mmol) of a compound (D336) as a white solid (yield: 59%). D336 had a sublimation temperature of 310° C., and it was confirmed that the sublimate D336 was glassy.

The compound was identified by FDMS measurement.

FDMS: 850

### Example 17 (Synthesis of Compound (D350))

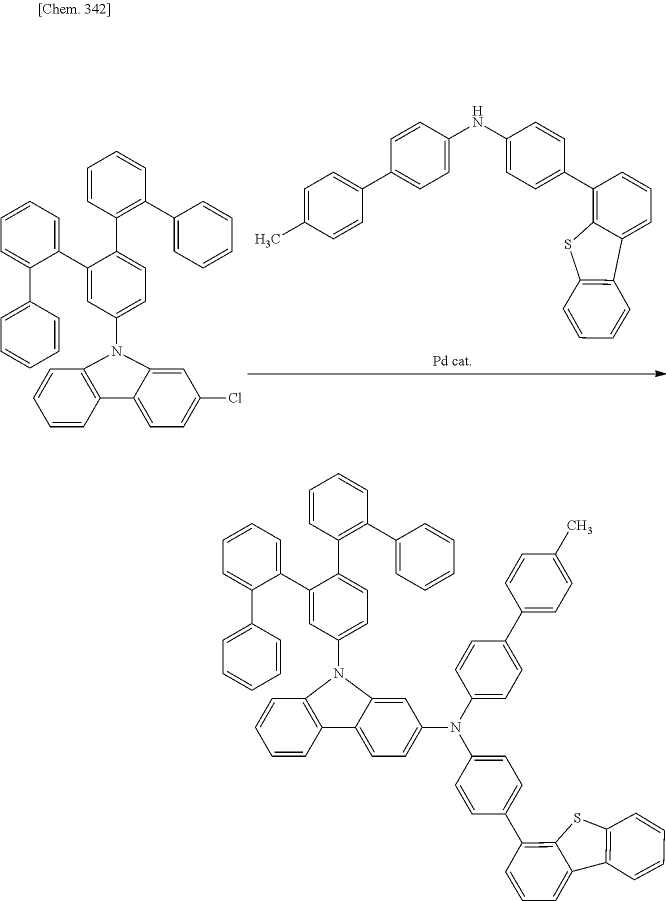

In a nitrogen stream, 3.0 g (5.2 mmol) of 9-([1,1:2′,1″:2″,1′″:2′″,1″″-quinquiphenyl]-4″-yl)-2-chloro-9H-carbazole prepared in Synthesis Example 18, 2.2 g (5.0 mmol) of N-(4-(dibenzo[b,d]thiophen-4-yl)phenyl)-4′-methyl-[1,1′-biphenyl]-4-amine, 0.62 g (6.5 mmol) of sodium-tert-butoxide, 20 mL of xylene, 11 mg (50 μmol) of palladium acetate and 0.12 g (0.15 mmol) of a 25% by weight xylene solution of tri(tert-butyl)phosphine were added to a 100-mL three-neck flask and were stirred at 140° C. for 22 hours. After cooling to room temperature, 22 mL of pure water was added thereto and stirred. An aqueous layer and an organic layer were then separated, and the organic layer was washed with saturated aqueous sodium chloride. The organic layer was dried over anhydrous magnesium sulfate and was then subjected to column chromatography with a small amount of silica gel to remove a polar component. The solvent was then distilled off under reduced pressure, and the resulting solid was recrystallized with a mixed solvent of toluene and butanol to isolate 3.0 g (3.0 mmol) of a compound (D350) as a white solid (yield: 60%). D350 had a sublimation temperature of 350° C., and it was confirmed that the sublimate D350 was glassy.

The compound was identified by FDMS measurement.

FDMS: 986

### Example 18 (Synthesis of Compound (D352))

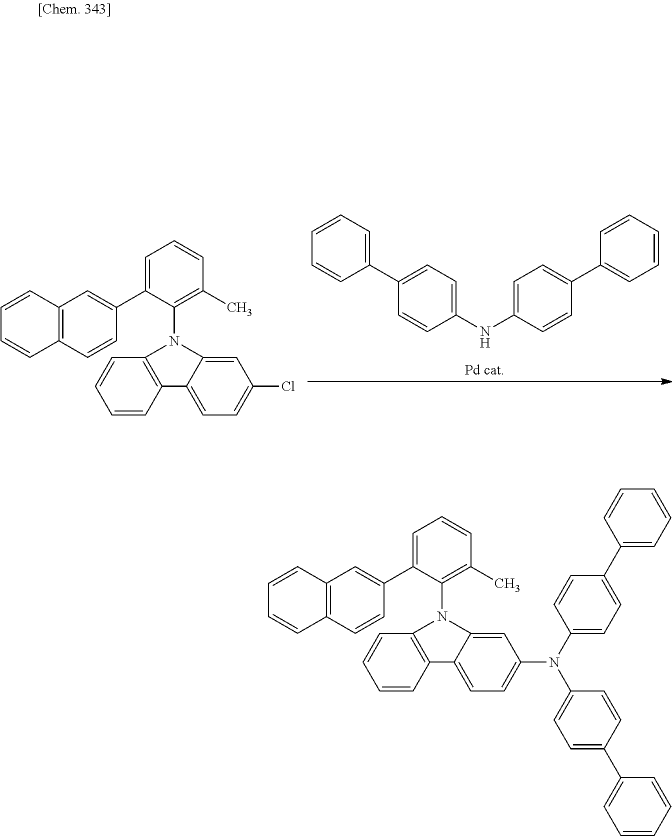 [SMILES: Cc1cccc(-c2ccc3ccccc3c2)c1-n1c2ccccc2c2ccc(Cl)cc21 ; c1ccc(-c2ccc(Nc3ccc(-c4ccccc4)cc3)cc2)cc1 ; Cc1cccc(-c2ccc3ccccc3c2)c1-n1c2ccccc2c2ccc(N(c3ccc(-c4ccccc4)cc3)c3ccc(-c4ccccc4)cc3)cc21]

In a nitrogen stream, 4.9 g (12 mmol) of 2-chloro-9-(2-methyl-6-(naphthalen-2-yl)phenyl)-9H-carbazole prepared in Synthesis Example 20, 3.6 g (11 mmol) of di([1,1′-biphenyl]-4-yl)amine, 1.4 g (15 mmol) of sodium-tert-butoxide, 20 mL of xylene, 25 mg (0.11 mmol) of palladium acetate and 0.27 g (0.34 mmol) of a 25% by weight xylene solution of tri(tert-butyl)phosphine were added to a 100-mL three-neck flask and were stirred at 140° C. for 22 hours. After cooling to room temperature, 22 mL of pure water was added thereto and stirred. An aqueous layer and an organic layer were then separated, and the organic layer was washed with saturated aqueous sodium chloride. The organic layer was dried over anhydrous magnesium sulfate and was then subjected to column chromatography with a small amount of silica gel to remove a polar component. The solvent was then distilled off under reduced pressure, and the resulting solid was recrystallized with a mixed solvent of toluene and butanol to isolate 5.8 g (8.3 mmol) of a compound (D352) as a white solid (yield: 74%). D352 had a sublimation temperature of 290° C., and it was confirmed that the sublimate D352 was glassy.

The compound was identified by FDMS measurement.

FDMS: 702

### Example 19 (Synthesis of Compound D357)

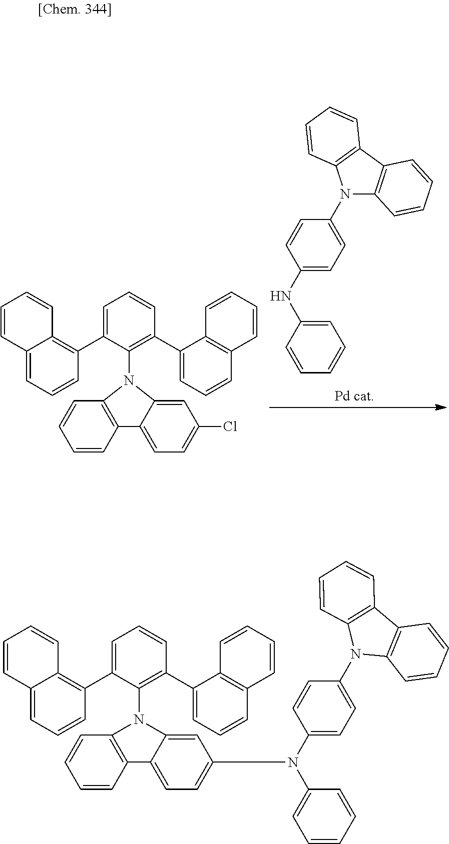

In a nitrogen stream, 5.5 g (10 mmol) of 2-chloro-9-(2,6-di(naphthalen-1-yl)phenyl)-9H-carbazole prepared in Synthesis Example 24, 3.3 g (9.9 mmol) of 4-(9H-carbazol-9-yl)-N-phenylaniline, 1.2 g (13 mmol) of sodium-tert-butoxide, 20 mL of xylene, 22 mg (99 μmol) of palladium acetate and 0.16 g (0.20 mmol) of a 25% by weight xylene solution of tri(tert-butyl)phosphine were added to a 100-mL three-neck flask and were stirred at 140° C. for 22 hours. After cooling to room temperature, 22 mL of pure water was added thereto and stirred. An aqueous layer and an organic layer were then separated, and the organic layer was washed with saturated aqueous sodium chloride. The organic layer was dried over anhydrous magnesium sulfate and was then subjected to column chromatography with a small amount of silica gel to remove a polar component. The solvent was then distilled off under reduced pressure, and the resulting solid was recrystallized with a mixed solvent of toluene and butanol to isolate 6.9 g (8.4 mmol) of a compound (D357) as a white solid (yield: 85%). D357 had a sublimation temperature of 330° C., and it was confirmed that the sublimate D357 was glassy.

The compound was identified by FDMS measurement.

FDMS: 827

### Example 20 (Synthesis of Compound (D360))

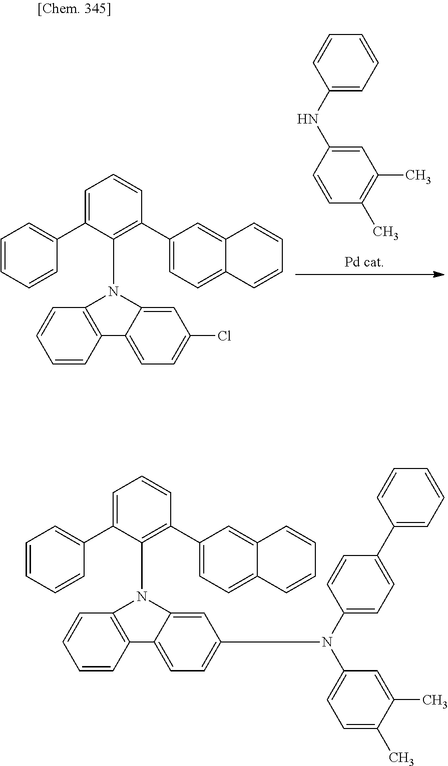 [SMILES: Clc1ccc2c3ccccc3n(-c3c(-c4ccccc4)cccc3-c3ccc4ccccc4c3)c2c1 ; Cc1ccc(Nc2ccccc2)cc1C ; Cc1ccc(N(c2ccc(-c3ccccc3)cc2)c2ccc3c4ccccc4n(-c4c(-c5ccccc5)cccc4-c4ccc5ccccc5c4)c3c2)cc1C]

In a nitrogen stream, 4.6 g (9.6 mmol) of 2-chloro-9-(3-(naphthalen-2-yl)-[1,1′-biphenyl]-2-yl)-9H-carbazole prepared in Synthesis Example 22, 2.5 g (9.1 mmol) of N-(3,4-dimethylphenyl)-[1,1′-biphenyl]-4-amine, 1.1 g (12 mmol) of sodium-tert-butoxide, 20 mL of xylene, 21 mg (91 μmol) of palladium acetate and 0.15 g (0.18 mmol) of a 25% by weight xylene solution of tri(tert-butyl)phosphine were added to a 100-mL three-neck flask and were stirred at 140° C. for 22 hours. After cooling to room temperature, 22 mL of pure water was added thereto and stirred. An aqueous layer and an organic layer were then separated, and the organic layer was washed with saturated aqueous sodium chloride. The organic layer was dried over anhydrous magnesium sulfate and was then subjected to column chromatography with a small amount of silica gel to remove a polar component. The solvent was then distilled off under reduced pressure, and the resulting solid was recrystallized with a mixed solvent of toluene and butanol to isolate 5.0 g (7.0 mmol) of a compound (D360) as a white solid (yield: 77%). D360 had a sublimation temperature of 280° C., and it was confirmed that the sublimate D360 was glassy.

The compound was identified by FDMS measurement.

FDMS: 716

### Example 21 (Synthesis of D429)

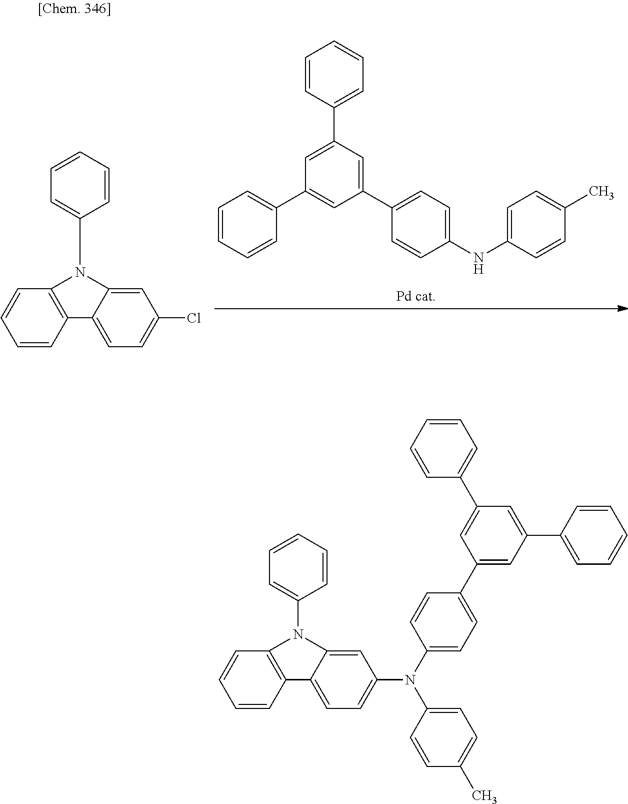 [SMILES: Clc1ccc2c3ccccc3n(-c3ccccc3)c2c1 ; Cc1ccc(Nc2ccc(-c3cc(-c4ccccc4)cc(-c4ccccc4)c3)cc2)cc1 ; Cc1ccc(N(c2ccc(-c3cc(-c4ccccc4)cc(-c4ccccc4)c3)cc2)c2ccc3c4ccccc4n(-c4ccccc4)c3c2)cc1]

In a nitrogen stream, 1.4 g (5.1 mmol) of 2-chloro-9-phenyl-9H-carbazole, 2.0 g (4.9 mmol) of 5′-phenyl-N-(p-tolyl)-[1,1:3′,1″-terphenyl]-4-amine, 0.61 g (6.3 mmol) of sodium-tert-butoxide, 20 mL of xylene, 11 mg (49 μmol) of palladium acetate and 79 mg (97 μmol) of a 25% by weight xylene solution of tri(tert-butyl)phosphine were added to a 100-mL three-neck flask and were stirred at 140° C. for 22 hours. After cooling to room temperature, 22 mL of pure water was added thereto and stirred. An aqueous layer and an organic layer were then separated, and the organic layer was washed with saturated aqueous sodium chloride. The organic layer was dried over anhydrous magnesium sulfate and was then subjected to column chromatography with a small amount of silica gel to remove a polar component. The solvent was then distilled off under reduced pressure, and the resulting solid was recrystallized with a mixed solvent of toluene and butanol to isolate 2.5 g (3.9 mmol) of a compound (D429) as a white solid (yield: 80%). D429 had a sublimation temperature of 330° C., and it was confirmed that the sublimate D429 was glassy.

The compound was identified by FDMS measurement.

FDMS: 652

### Example 22 (Synthesis of Compound (D657))

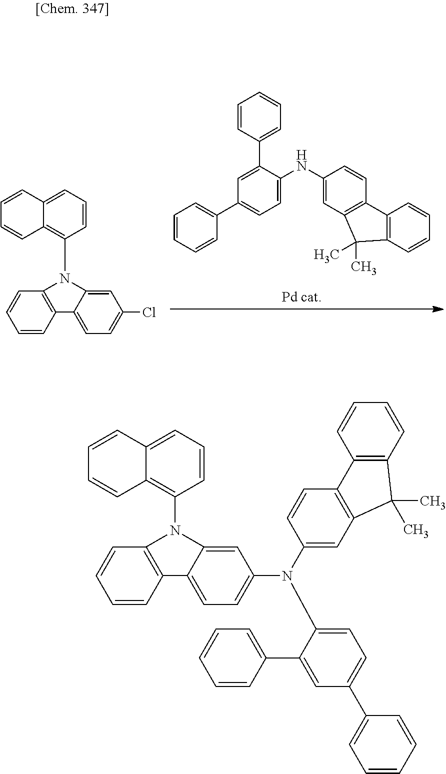 [SMILES: Clc1ccc2c3ccccc3n(-c3cccc4ccccc34)c2c1 ; CC1(C)c2ccccc2-c2ccc(Nc3ccc(-c4ccccc4)cc3-c3ccccc3)cc21 ; CC1(C)c2ccccc2-c2ccc(N(c3ccc4c5ccccc5n(-c5cccc6ccccc56)c4c3)c3ccc(-c4ccccc4)cc3-c3ccccc3)cc21]

In a nitrogen stream, 2.0 g (6.2 mmol) of 2-chloro-9-(naphthalen-1-yl)-9H-carbazole, 2.6 g (5.9 mmol) of N-([1,1′:3′,1″-terphenyl]-4′-yl)-9,9-dimethyl-9H-fluorene-2-amine, 0.74 g (7.7 mmol) of sodium-tert-butoxide, 20 mL of xylene, 13 mg (59 μmol) of palladium acetate and 96 mg (0.12 mmol) of a 25% by weight xylene solution of tri(tert-butyl)phosphine were added to a 100-mL three-neck flask and were stirred at 140° C. for 22 hours. After cooling to room temperature, 22 mL of pure water was added thereto and stirred. An aqueous layer and an organic layer were then separated, and the organic layer was washed with saturated aqueous sodium chloride. The organic layer was dried over anhydrous magnesium sulfate and was then subjected to column chromatography with a small amount of silica gel to remove a polar component. The solvent was then distilled off under reduced pressure, and the resulting solid was recrystallized with a mixed solvent of toluene and butanol to isolate 3.2 g (4.3 mmol) of a compound (D657) as a white solid (yield: 73%). D657 had a sublimation temperature of 270° C., and it was confirmed that the sublimate D657 was glassy.

The compound was identified by FDMS measurement.

FDMS: 728

### Example 23 (Synthesis of Compound (D757))

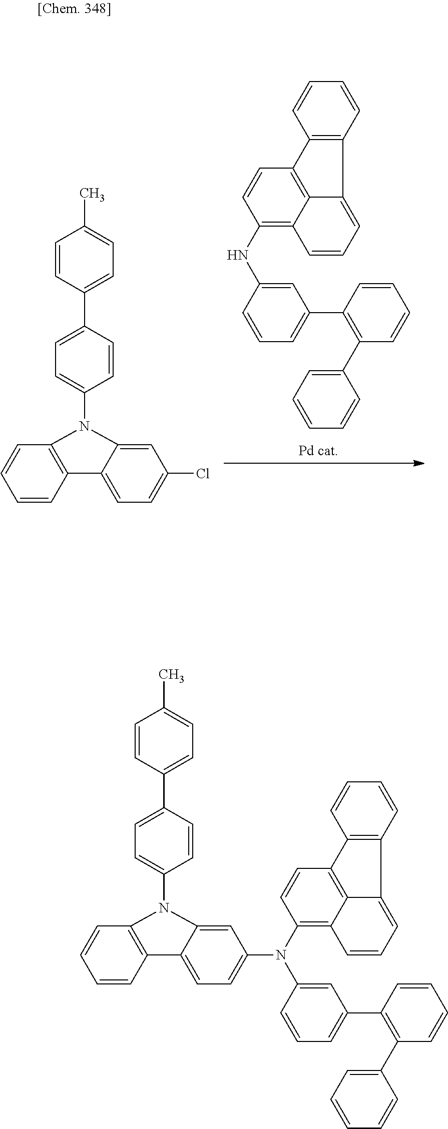 [SMILES: Cc1ccc(-c2ccc(-n3c4ccccc4c4ccc(Cl)cc43)cc2)cc1 ; c1ccc(-c2ccccc2-c2cccc(Nc3ccc4c5c(cccc35)-c3ccccc3-4)c2)cc1 ; Cc1ccc(-c2ccc(-n3c4ccccc4c4ccc(N(c5cccc(-c6ccccc6-c6ccccc6)c5)c5ccc6c7c(cccc57)-c5ccccc5-6)cc43)cc2)cc1]

In a nitrogen stream, 2.1 g (5.7 mmol) of 2-chloro-9-(4′-methyl-[1,1′-biphenyl]-4-yl)-9H-carbazole, 2.4 g (5.4 mmol) of N-([1,1′:2′,1″-terphenyl]-3-yl)fluoranthene-3-amine, 0.67 g (7.0 mmol) of sodium-tert-butoxide, 20 mL of xylene, 12 mg (54 μmol) of palladium acetate and 87 mg (0.11 mmol) of a 25% by weight xylene solution of tri(tert-butyl)phosphine were added to a 100-mL three-neck flask and were stirred at 140° C. for 22 hours. After cooling to room temperature, 22 mL of pure water was added thereto and stirred. An aqueous layer and an organic layer were then separated, and the organic layer was washed with saturated aqueous sodium chloride. The organic layer was dried over anhydrous magnesium sulfate and was then subjected to column chromatography with a small amount of silica gel to remove a polar component. The solvent was then distilled off under reduced pressure, and the resulting solid was recrystallized with a mixed solvent of toluene and butanol to isolate 3.1 g (4.0 mmol) of a compound (D757) as a white solid (yield: 74%). D757 had a sublimation temperature of 330° C., and it was confirmed that the sublimate D757 was glassy.

The compound was identified by FDMS measurement.

FDMS: 776

### Example 24 (Synthesis of Compound (D805))

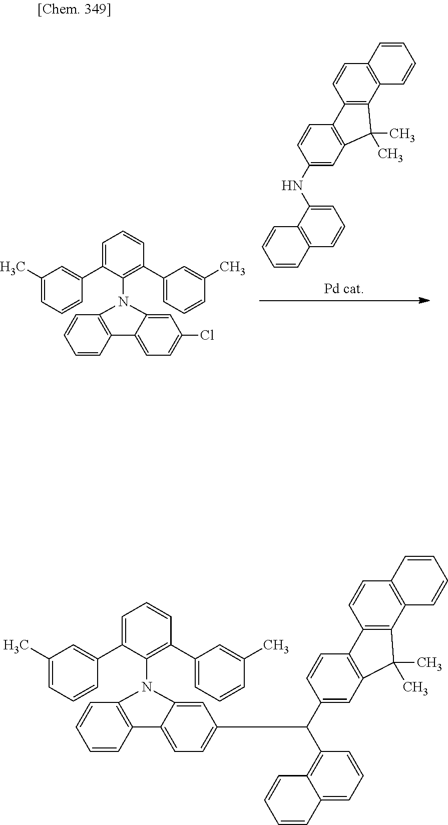

In a nitrogen stream, 5.0 g (11 mmol) of 2-chloro-9-(3,3″-dimethyl-[1,1′:3′,1″-terphenyl]-2′-yl)-9H-carbazole prepared in Synthesis Example 26, 4.0 g (10 mmol) of 11,11-dimethyl-N-(naphthalen-1-yl)-11H-benzo[a]fluorene-9-amine, 1.3 g (13 mmol) of sodium-tert-butoxide, 20 mL of xylene, 23 mg (0.10 mmol) of palladium acetate and 0.17 g (0.21 mmol) of a 25% by weight xylene solution of tri(tert-butyl)phosphine were added to a 100-mL three-neck flask and were stirred at 140° C. for 22 hours. After cooling to room temperature, 22 mL of pure water was added thereto and stirred. An aqueous layer and an organic layer were then separated, and the organic layer was washed with saturated aqueous sodium chloride. The organic layer was dried over anhydrous magnesium sulfate and was then subjected to column chromatography with a small amount of silica gel to remove a polar component. The solvent was then distilled off under reduced pressure, and the resulting solid was recrystallized with a mixed solvent of toluene and butanol to isolate 6.7 g (8.3 mmol) of a compound (D805) as a white solid (yield: 80%). D805 had a sublimation temperature of 280° C., and it was confirmed that the sublimate D805 was glassy.

The compound was identified by FDMS measurement.

FDMS: 805

### Example 25 (Synthesis of Compound (D818))

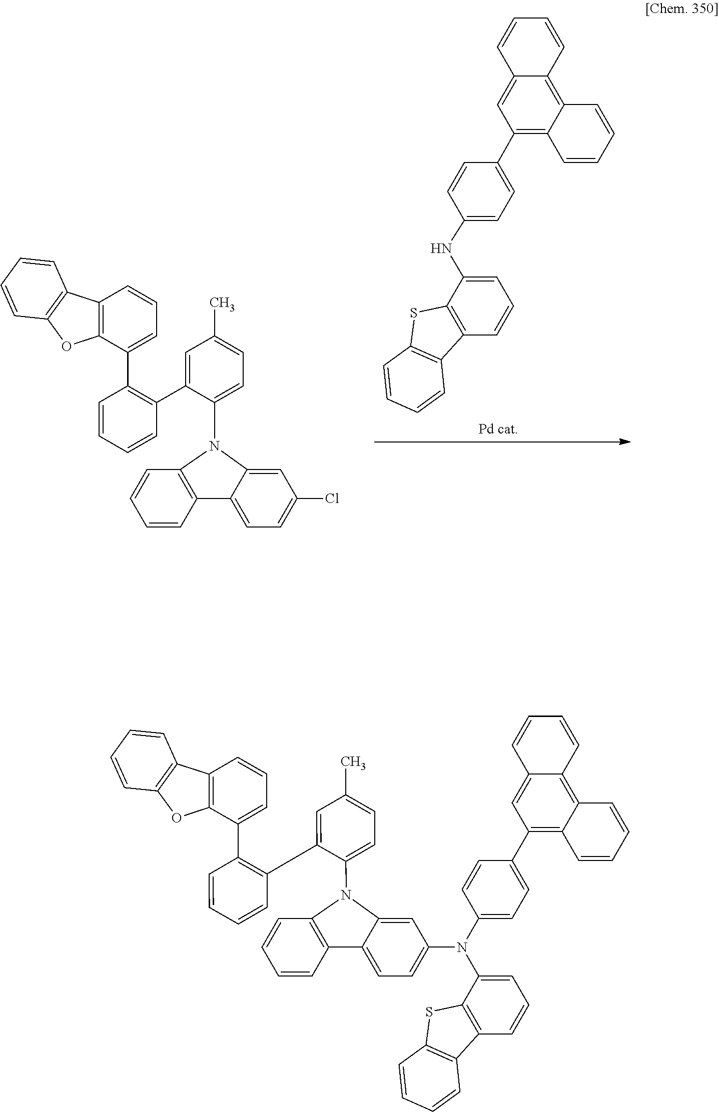 [SMILES: Cc1ccc(-n2c3ccccc3c3ccc(Cl)cc32)c(-c2ccccc2-c2cccc3c2oc2ccccc23)c1 ; c1ccc2c(c1)cc(-c1ccc(Nc3cccc4c3sc3ccccc34)cc1)c1ccccc12 ; Cc1ccc(-n2c3ccccc3c3ccc(N(c4ccc(-c5cc6ccccc6c6ccccc56)cc4)c4cccc5c4sc4ccccc45)cc32)c(-c2ccccc2-c2cccc3c2oc2ccccc23)c1]

In a nitrogen stream, 4.0 g (7.4 mmol) of 2-chloro-9-(2′-(dibenzo[b,d]furan-4-yl)-5-methyl-[1,1′-biphenyl]-2-yl)-9H-carbazole prepared in Synthesis Example 27, 3.2 g (7.1 mmol) of N-(4-(phenanthren-9-yl)phenyl)dibenzo[b,d]thiophene-4-amine, 0.89 g (9.2 mmol) of sodium-tert-butoxide, 20 mL of xylene, 16 mg (71 μmol) of palladium acetate and 0.11 g (0.14 mmol) of a 25% by weight xylene solution of tri(tert-butyl)phosphine were added to a 100-mL three-neck flask and were stirred at 140° C. for 22 hours. After cooling to room temperature, 22 mL of pure water was added thereto and stirred. An aqueous layer and an organic layer were then separated, and the organic layer was washed with saturated aqueous sodium chloride. The organic layer was dried over anhydrous magnesium sulfate and was then subjected to column chromatography with a small amount of silica gel to remove a polar component. The solvent was then distilled off under reduced pressure, and the resulting solid was recrystallized with a mixed solvent of toluene and butanol to isolate 4.8 g (5.0 mmol) of a compound (D818) as a white solid (yield: 71%). D818 had a sublimation temperature of 350° C., and it was confirmed that the sublimate D818 was glassy.

The compound was identified by FDMS measurement.

FDMS: 948

### Example 26 (Synthesis of Compound (D844))

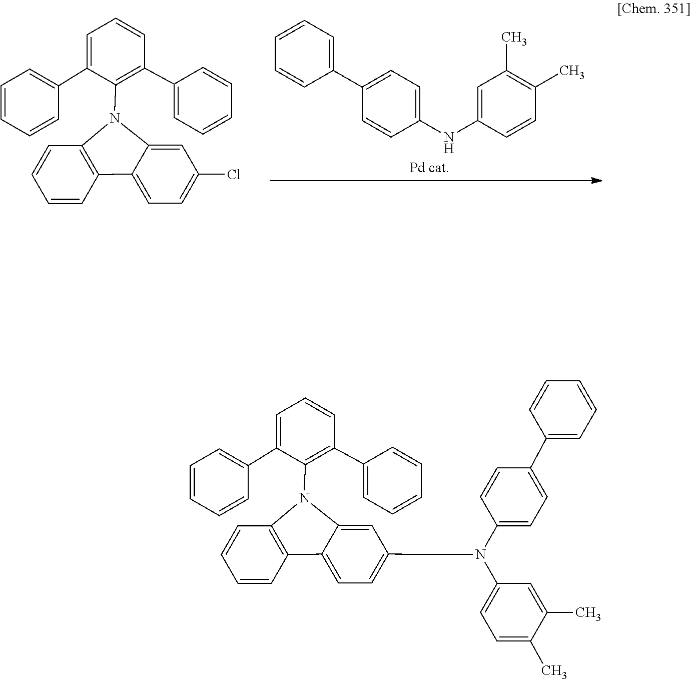

In a nitrogen stream, 3.3 g (7.7 mmol) of 9-([1,1′:3′,1″-terphenyl]-2′-yl)-2-chloro-9H-carbazole prepared in Synthesis Example 5, 2.0 g (7.3 mmol) of N-(3,4-dimethylphenyl)-[1,1′-biphenyl]-4-amine, 0.91 g (9.5 mmol) of sodium-tert-butoxide, 20 mL of xylene, 16 mg (73 μmol) of palladium acetate and 0.12 g (0.15 mmol) of a 25% by weight xylene solution of tri(tert-butyl)phosphine were added to a 100-mL three-neck flask and were stirred at 140° C. for 22 hours. After cooling to room temperature, 22 mL of pure water was added thereto and stirred. An aqueous layer and an organic layer were then separated, and the organic layer was washed with saturated aqueous sodium chloride. The organic layer was dried over anhydrous magnesium sulfate and was then subjected to column chromatography with a small amount of silica gel to remove a polar component. The solvent was then distilled off under reduced pressure, and the resulting solid was recrystallized with a mixed solvent of toluene and butanol to isolate 3.9 g (5.9 mmol) of a compound (D844) as a white solid (yield: 80%). D844 had a sublimation temperature of 270° C., and it was confirmed that the sublimate D844 was glassy.

The compound was identified by FDMS measurement.

FDMS: 666

### Example 27 (Synthesis of Compound (D850))

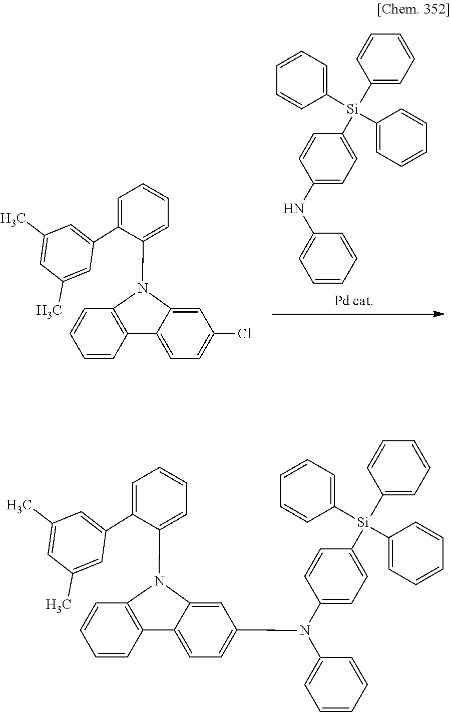 [SMILES: Cc1cc(C)cc(-c2ccccc2-n2c3ccccc3c3ccc(Cl)cc32)c1 ; c1ccc(Nc2ccc([Si](c3ccccc3)(c3ccccc3)c3ccccc3)cc2)cc1 ; Cc1cc(C)cc(-c2ccccc2-n2c3ccccc3c3ccc(N(c4ccccc4)c4ccc([Si](c5ccccc5)(c5ccccc5)c5ccccc5)cc4)cc32)c1]

In a nitrogen stream, 3.8 g (9.8 mmol) of 2-chloro-9-(3′,5′-dimethyl-[1,1′-biphenyl]-2-yl)-9H-carbazole prepared in Synthesis Example 29, 4.0 g (9.4 mmol) of N-phenyl-4-(triphenylsilyl)aniline, 1.2 g (12 mmol) of sodium-tert-butoxide, 20 mL of xylene, 21 mg (94 μmol) of palladium acetate and 0.15 g (0.19 mmol) of a 25% by weight xylene solution of tri(tert-butyl)phosphine were added to a 100-mL three-neck flask and were stirred at 140° C. for 22 hours. After cooling to room temperature, 22 mL of pure water was added thereto and stirred. An aqueous layer and an organic layer were then separated, and the organic layer was washed with saturated aqueous sodium chloride. The organic layer was dried over anhydrous magnesium sulfate and was then subjected to column chromatography with a small amount of silica gel to remove a polar component. The solvent was then distilled off under reduced pressure, and the resulting solid was recrystallized with a mixed solvent of toluene and butanol to isolate 4.4 g (5.7 mmol) of a compound (D850) as a white solid (yield: 61%). D850 had a sublimation temperature of 310° C., and it was confirmed that the sublimate D850 was glassy.

The compound was identified by FDMS measurement.

FDMS: 772

### Example 28 (Synthesis of Compound (D853))

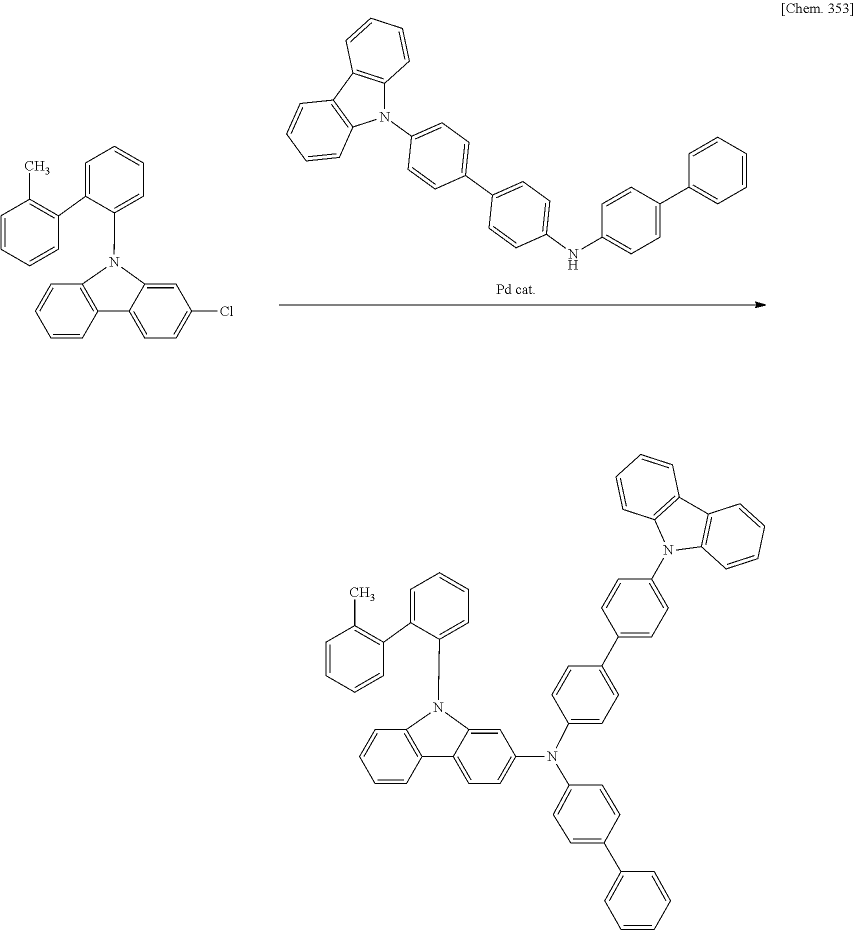

In a nitrogen stream, 2.6 g (7.1 mmol) of 2-chloro-9-(2′-methyl-[1,1′-biphenyl]-2-yl)-9H-carbazole prepared in Synthesis Example 30, 3.3 g (6.8 mmol) of N-([1,1′-biphenyl]-4-yl)-4′-(9H-carbazol-9-yl)-[1,1′-biphenyl]-4-amine, 0.85 g (8.8 mmol) of sodium-tert-butoxide, 20 mL of xylene, 15 mg (68 μmol) of palladium acetate and 0.11 g (0.14 mmol) of a 25% by weight xylene solution of tri(tert-butyl)phosphine were added to a 100-mL three-neck flask and were stirred at 140° C. for 22 hours. After cooling to room temperature, 22 mL of pure water was added thereto and stirred. An aqueous layer and an organic layer were then separated, and the organic layer was washed with saturated aqueous sodium chloride. The organic layer was dried over anhydrous magnesium sulfate and was then subjected to column chromatography with a small amount of silica gel to remove a polar component. The solvent was then distilled off under reduced pressure, and the resulting solid was recrystallized with a mixed solvent of toluene and butanol to isolate 4.2 g (5.2 mmol) of a compound (D853) as a white solid (yield: 76%). D853 had a sublimation temperature of 330° C., and it was confirmed that the sublimate D853 was glassy.

The compound was identified by FDMS measurement.

FDMS: 817

### Example 29 (Synthesis of Compound (D859))

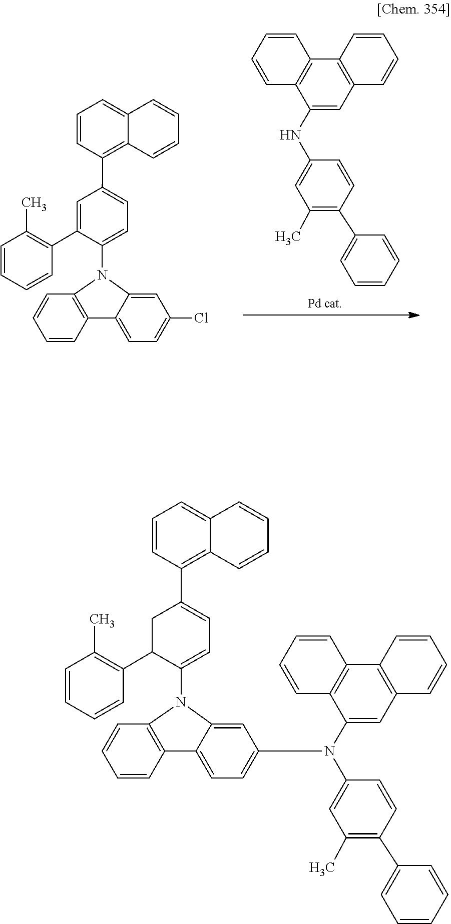 [SMILES: Cc1ccccc1-c1cc(-c2cccc3ccccc23)ccc1-n1c2ccccc2c2ccc(Cl)cc21 ; Cc1cc(Nc2cc3ccccc3c3ccccc23)ccc1-c1ccccc1 ; Cc1cc(N(c2ccc3c4ccccc4n(C4=CC=C(c5cccc6ccccc56)CC4c4ccccc4C)c3c2)c2cc3ccccc3c3ccccc23)ccc1-c1ccccc1]

In a nitrogen stream, 3.6 g (7.6 mmol) of 2-chloro-9-(2′-methyl-5-(naphthalen-1-yl)-[1,1′-biphenyl]-2-yl)-9H-carbazole prepared in Synthesis Example 32, 2.6 g (7.2 mmol) of N-(2-methyl-[1,1′-biphenyl]-4-yl)phenanthrene-9-amine, 0.90 g (9.4 mmol) of sodium-tert-butoxide, 20 mL of xylene, 16 mg (72 μmol) of palladium acetate and 0.12 g (0.14 mmol) of a 25% by weight xylene solution of tri(tert-butyl)phosphine were added to a 100-mL three-neck flask and were stirred at 140° C. for 22 hours. After cooling to room temperature, 22 mL of pure water was added thereto and stirred. An aqueous layer and an organic layer were then separated, and the organic layer was washed with saturated aqueous sodium chloride. The organic layer was dried over anhydrous magnesium sulfate and was then subjected to column chromatography with a small amount of silica gel to remove a polar component. The solvent was then distilled off under reduced pressure, and the resulting solid was recrystallized with a mixed solvent of toluene and butanol to isolate 3.8 g (4.6 mmol) of a compound (D859) as a white solid (yield: 64%). D859 had a sublimation temperature of 335° C., and it was confirmed that the sublimate D859 was glassy.

The compound was identified by FDMS measurement.

FDMS: 816

### Example 30 (Synthesis of Compound (E103))

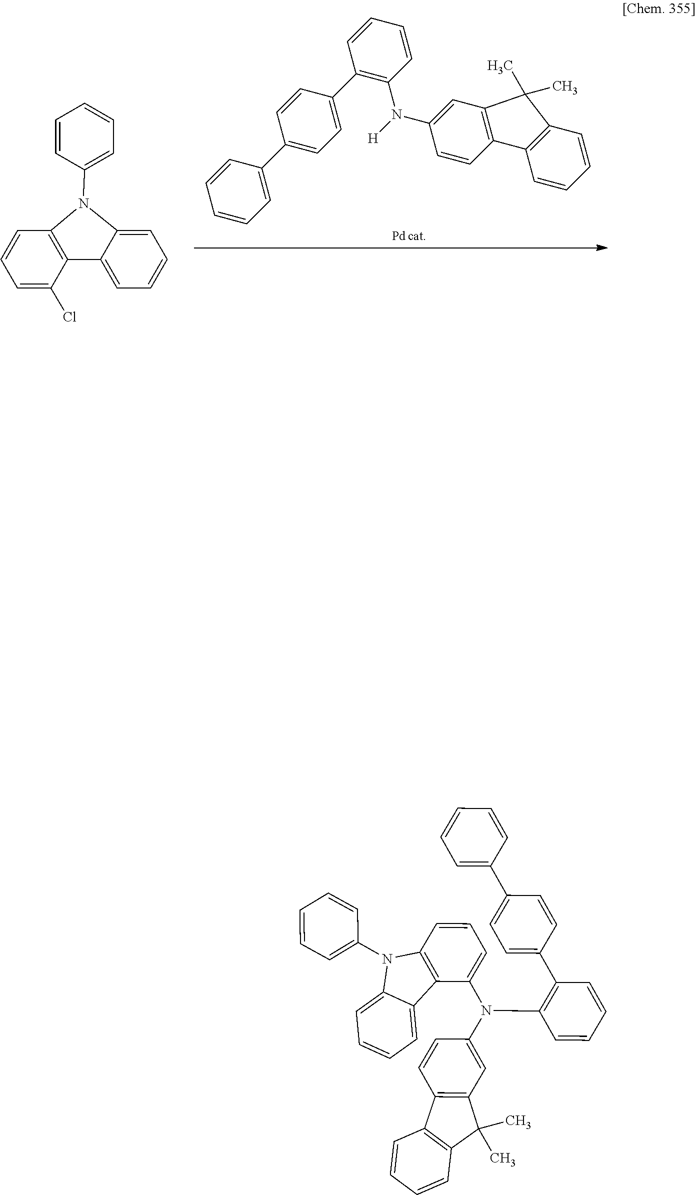 [SMILES: Clc1cccc2c1c1ccccc1n2-c1ccccc1 ; CC1(C)c2ccccc2-c2ccc(Nc3ccccc3-c3ccc(-c4ccccc4)cc3)cc21 ; CC1(C)c2ccccc2-c2ccc(N(c3ccccc3-c3ccc(-c4ccccc4)cc3)c3cccc4c3c3ccccc3n4-c3ccccc3)cc21]

In a nitrogen stream, 2.0 g (7.2 mmol) of 4-chloro-9-phenyl-9H-carbazole, 3.0 g (6.9 mmol) of N-([1,1′:4′,1″-terphenyl]-2-yl)-9,9-dimethyl-9H-fluorene-2-amine, 0.86 g (8.9 mmol) of sodium-tert-butoxide, 20 mL of xylene, 15 mg (69 μmol) of palladium acetate and 0.11 g (0.14 mmol) of a 25% by weight xylene solution of tri(tert-butyl)phosphine were added to a 100-mL three-neck flask and were stirred at 140° C. for 22 hours. After cooling to room temperature, 22 mL of pure water was added thereto and stirred. An aqueous layer and an organic layer were then separated, and the organic layer was washed with saturated aqueous sodium chloride. The organic layer was dried over anhydrous magnesium sulfate and was then subjected to column chromatography with a small amount of silica gel to remove a polar component. The solvent was then distilled off under reduced pressure, and the resulting solid was recrystallized with a mixed solvent of toluene and butanol to isolate 3.8 g (5.6 mmol) of a compound (E103) as a white solid (yield: 81%). E103 had a sublimation temperature of 300° C., and it was confirmed that the sublimate E103 was glassy.

The compound was identified by FDMS measurement.

FDMS: 678

### Example 31 (Synthesis of Compound (E107))

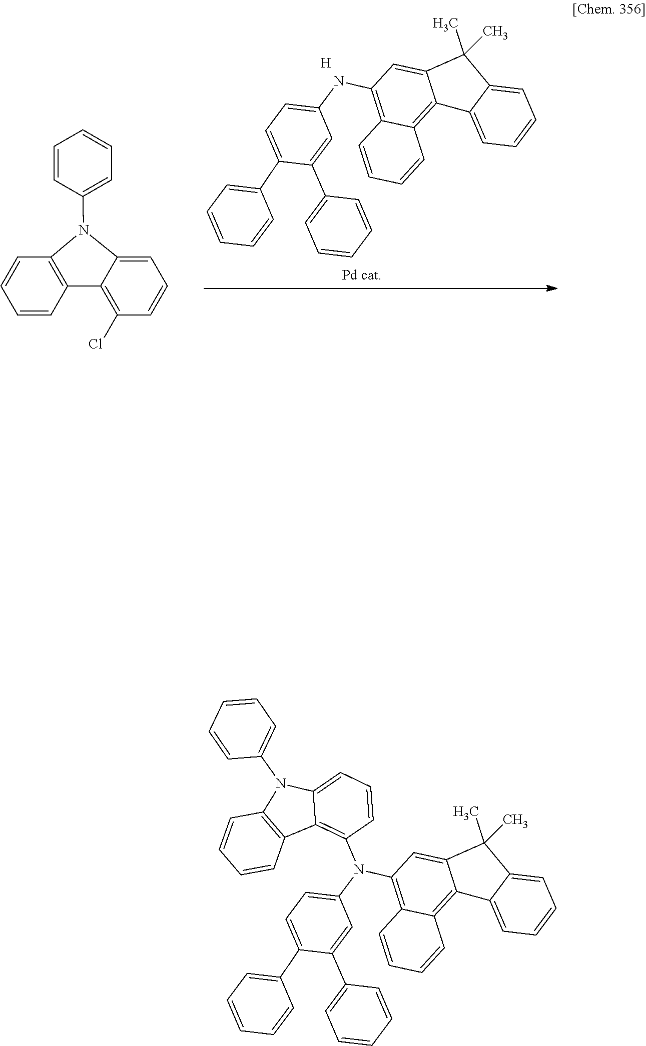

In a nitrogen stream, 1.8 g (6.5 mmol) of 4-chloro-9-phenyl-9H-carbazole, 3.0 g (6.2 mmol) of N-([1,1′:2′,1″-terphenyl]-4′-yl)-7,7-dimethyl-7H-benzo[c]fluorene-5-amine, 0.77 g (8.0 mmol) of sodium-tert-butoxide, 20 mL of xylene, 14 mg (62 μmol) of palladium acetate and 0.10 g (0.12 mmol) of a 25% by weight xylene solution of tri(tert-butyl)phosphine were added to a 100-mL three-neck flask and were stirred at 140° C. for 22 hours. After cooling to room temperature, 22 mL of pure water was added thereto and stirred. An aqueous layer and an organic layer were then separated, and the organic layer was washed with saturated aqueous sodium chloride. The organic layer was dried over anhydrous magnesium sulfate and was then subjected to column chromatography with a small amount of silica gel to remove a polar component. The solvent was then distilled off under reduced pressure, and the resulting solid was recrystallized with a mixed solvent of toluene and butanol to isolate 2.8 g (3.9 mmol) of a compound (E107) as a white solid (yield: 63%). E107 had a sublimation temperature of 310° C., and it was confirmed that the sublimate E107 was glassy.

The compound was identified by FDMS measurement.

FDMS: 728

### Example 32 (Synthesis of Compound (E111))

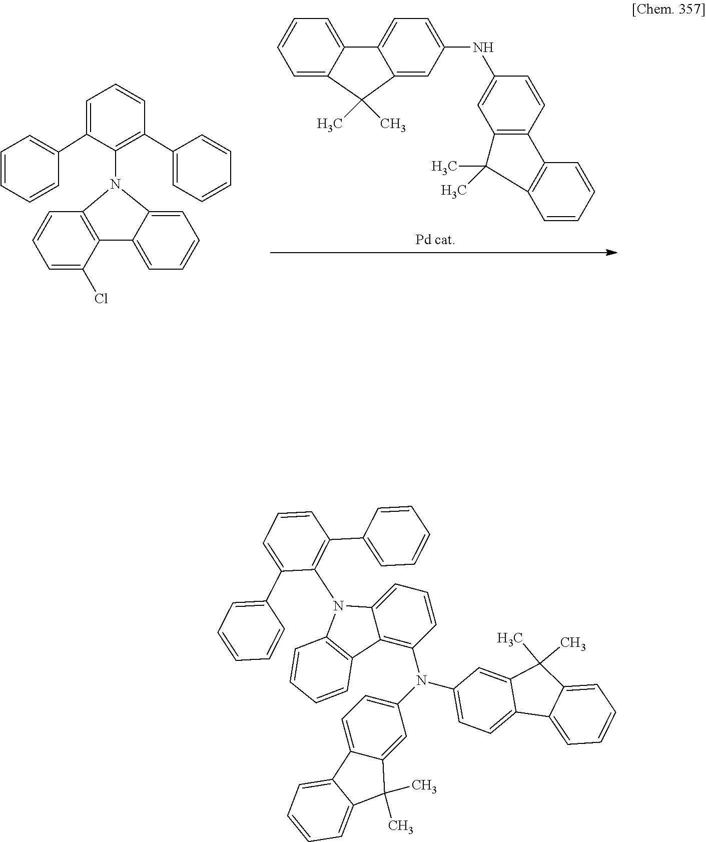 [SMILES: Clc1cccc2c1c1ccccc1n2-c1c(-c2ccccc2)cccc1-c1ccccc1 ; CC1(C)c2ccccc2-c2ccc(Nc3ccc4c(c3)C(C)(C)c3ccccc3-4)cc21 ; CC1(C)c2ccccc2-c2ccc(N(c3ccc4c(c3)C(C)(C)c3ccccc3-4)c3cccc4c3c3ccccc3n4-c3c(-c4ccccc4)cccc3-c3ccccc3)cc21]

In a nitrogen stream, 2.8 g (6.5 mmol) of 9-([1,1′:3′,1″-terphenyl]-2′-yl)-4-chloro-9H-carbazole, 2.5 g (6.2 mmol) of bis(9,9-dimethyl-9H-fluoren-2-yl)amine, 0.78 g (8.1 mmol) of sodium-tert-butoxide, 20 mL of xylene, 14 mg (62 μmol) of palladium acetate and 0.10 g (0.12 mmol) of a 25% by weight xylene solution of tri(tert-butyl)phosphine were added to a 100-mL three-neck flask and were stirred at 140° C. for 22 hours. After cooling to room temperature, 22 mL of pure water was added thereto and stirred. An aqueous layer and an organic layer were then separated, and the organic layer was washed with saturated aqueous sodium chloride. The organic layer was dried over anhydrous magnesium sulfate and was then subjected to column chromatography with a small amount of silica gel to remove a polar component. The solvent was then distilled off under reduced pressure, and the resulting solid was recrystallized with a mixed solvent of toluene and butanol to isolate 4.0 g (5.0 mmol) of a compound (E111) as a white solid (yield: 80%). E111 had a sublimation temperature of 315° C., and it was confirmed that the sublimate E111 was glassy.

The compound was identified by FDMS measurement.

FDMS: 794

### Example 33 (Synthesis of Compound (E161))

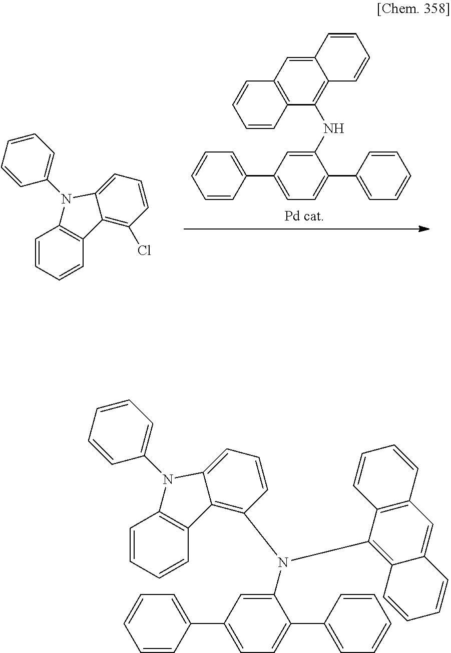 [SMILES: Clc1cccc2c1c1ccccc1n2-c1ccccc1 ; c1ccc(-c2ccc(-c3ccccc3)c(Nc3c4ccccc4cc4ccccc34)c2)cc1 ; c1ccc(-c2ccc(-c3ccccc3)c(N(c3c4ccccc4cc4ccccc34)c3cccc4c3c3ccccc3n4-c3ccccc3)c2)cc1]

In a nitrogen stream, 1.8 g (6.5 mmol) of 4-chloro-9-phenyl-9H-carbazole, 2.6 g (6.2 mmol) of N-([1,1′:4′,1″-terphenyl]-2′-yl)anthracen-9-amine, 0.77 g (8.0 mmol) of sodium-tert-butoxide, 20 mL of xylene, 14 mg (62 μmol) of palladium acetate and 0.10 g (0.12 mmol) of a 25% by weight xylene solution of tri(tert-butyl)phosphine were added to a 100-mL three-neck flask and were stirred at 140° C. for 22 hours. After cooling to room temperature, 22 mL of pure water was added thereto and stirred. An aqueous layer and an organic layer were then separated, and the organic layer was washed with saturated aqueous sodium chloride. The organic layer was dried over anhydrous magnesium sulfate and was then subjected to column chromatography with a small amount of silica gel to remove a polar component. The solvent was then distilled off under reduced pressure, and the resulting solid was recrystallized with a mixed solvent of toluene and butanol to isolate 2.7 g (4.1 mmol) of a compound (E161) as a white solid (yield: 66%). E161 had a sublimation temperature of 285° C., and it was confirmed that the sublimate E161 was glassy.

The compound was identified by FDMS measurement.

FDMS: 662

### Example 34 (Synthesis of Compound (E169))

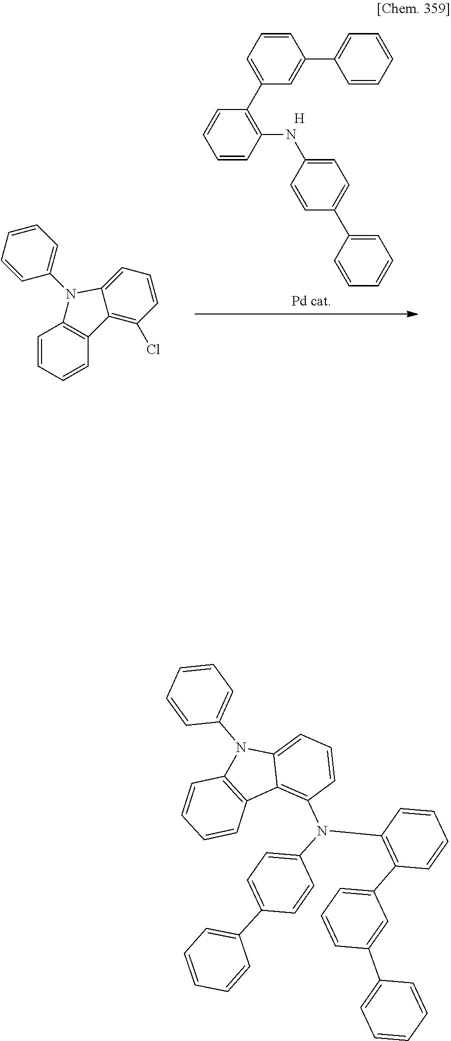

In a nitrogen stream, 2.2 g (7.9 mmol) of 4-chloro-9-phenyl-9H-carbazole, 3.0 g (7.5 mmol) of N-([1,1′-biphenyl]-4-yl)-[1,1:3′,1″-terphenyl]-2-amine, 0.94 g (9.8 mmol) of sodium-tert-butoxide, 20 mL of xylene, 17 mg (75 μmol) of palladium acetate and 0.12 g (0.15 mmol) of a 25% by weight xylene solution of tri(tert-butyl)phosphine were added to a 100-mL three-neck flask and were stirred at 140° C. for 22 hours. After cooling to room temperature, 22 mL of pure water was added thereto and stirred. An aqueous layer and an organic layer were then separated, and the organic layer was washed with saturated aqueous sodium chloride. The organic layer was dried over anhydrous magnesium sulfate and was then subjected to column chromatography with a small amount of silica gel to remove a polar component. The solvent was then distilled off under reduced pressure, and the resulting solid was recrystallized with a mixed solvent of toluene and butanol to isolate 3.4 g (5.4 mmol) of a compound (E169) as a white solid (yield: 71%). E169 had a sublimation temperature of 300° C., and it was confirmed that the sublimate E169 was glassy.

The compound was identified by FDMS measurement.

FDMS: 638

### Example 35 (Synthesis of Compound (E179))

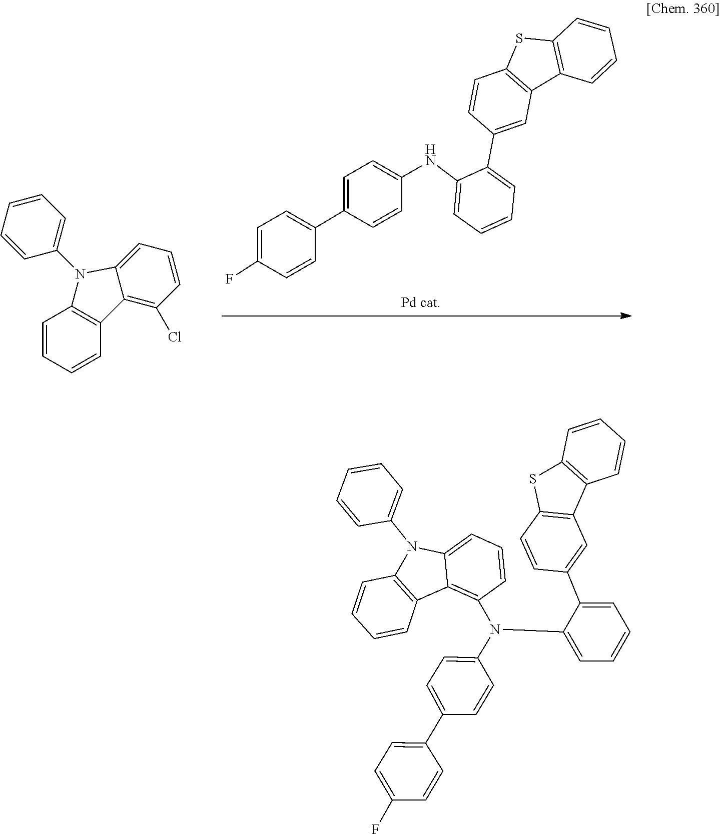

In a nitrogen stream, 2.3 g (8.2 mmol) of 4-chloro-9-phenyl-9H-carbazole, 3.5 g (7.9 mmol) of N-(2-(dibenzo[b,d]thiophen-2-yl)phenyl)-4′-fluoro-[1,1′-biphenyl]-4-amine, 0.98 g (10 mmol) of sodium-tert-butoxide, 20 mL of xylene, 18 mg (79 μmol) of palladium acetate and 0.13 g (0.16 mmol) of a 25% by weight xylene solution of tri(tert-butyl)phosphine were added to a 100-mL three-neck flask and were stirred at 140° C. for 22 hours. After cooling to room temperature, 22 mL of pure water was added thereto and stirred. An aqueous layer and an organic layer were then separated, and the organic layer was washed with saturated aqueous sodium chloride. The organic layer was dried over anhydrous magnesium sulfate and was then subjected to column chromatography with a small amount of silica gel to remove a polar component. The solvent was then distilled off under reduced pressure, and the resulting solid was recrystallized with a mixed solvent of toluene and butanol to isolate 2.9 g (4.2 mmol) of a compound (E179) as a white solid (yield: 54%). E179 had a sublimation temperature of 305° C., and it was confirmed that the sublimate E179 was glassy.

The compound was identified by FDMS measurement.

FDMS: 686

### Example 36 (Synthesis of Compound (E228))

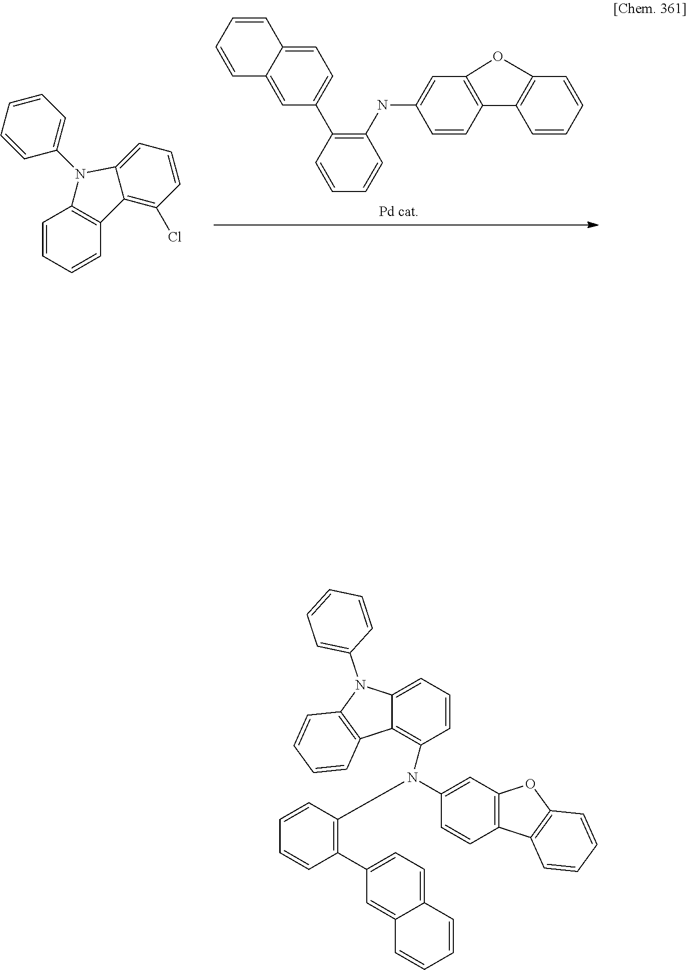

In a nitrogen stream, 2.6 g (9.3 mmol) of 4-chloro-9-phenyl-9H-carbazole, 3.4 g (8.8 mmol) of N-(2-(naphthalen-2-yl)phenyl)dibenzo[b,d]furan-3-amine, 1.1 g (11 mmol) of sodium-tert-butoxide, 20 mL of xylene, 20 mg (88 μmol) of palladium acetate and 0.14 g (0.18 mmol) of a 25% by weight xylene solution of tri(tert-butyl)phosphine were added to a 100-mL three-neck flask and were stirred at 140° C. for 22 hours. After cooling to room temperature, 22 mL of pure water was added thereto and stirred. An aqueous layer and an organic layer were then separated, and the organic layer was washed with saturated aqueous sodium chloride. The organic layer was dried over anhydrous magnesium sulfate and was then subjected to column chromatography with a small amount of silica gel to remove a polar component. The solvent was then distilled off under reduced pressure, and the resulting solid was recrystallized with a mixed solvent of toluene and butanol to isolate 4.3 g (6.8 mmol) of a compound (E228) as a white solid (yield: 77%). E228 had a sublimation temperature of 300° C., and it was confirmed that the sublimate E228 was glassy.

The compound was identified by FDMS measurement.

FDMS: 626

### Example 37 (Synthesis of Compound (E264))

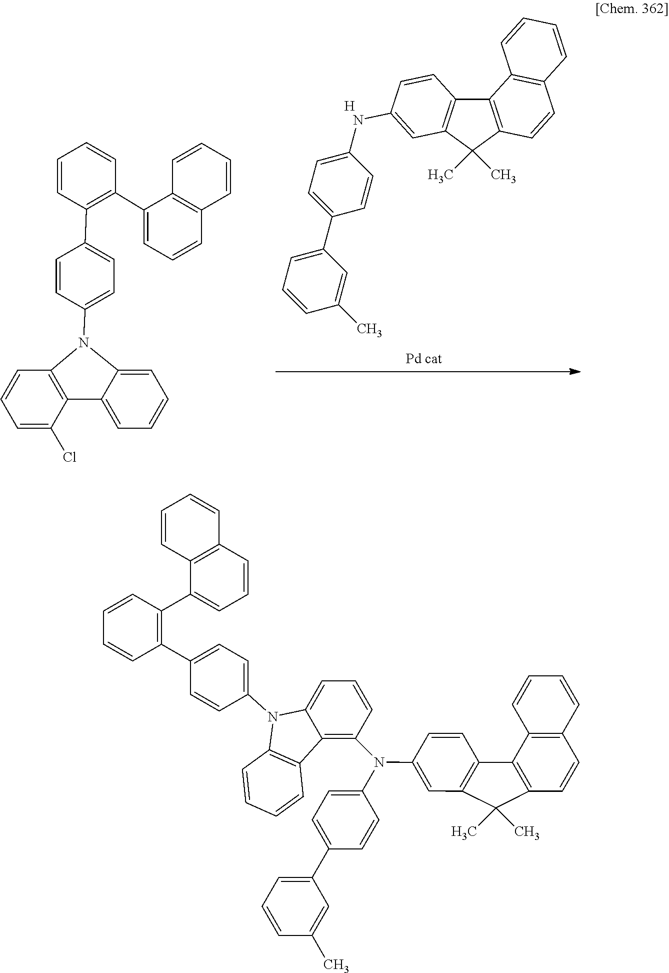

In a nitrogen stream, 2.4 g (4.9 mmol) of 4-chloro-9-(2′-(naphthalen-1-yl)-[1,1′-biphenyl]-4-yl)-9H-carbazole prepared in Synthesis Example 33, 2.0 g (4.7 mmol) of 7,7-dimethyl-N-(3′-methyl-[1,1′-biphenyl]-4-yl)-7H-benzo[c]fluorene-9-amine, 0.59 g (6.1 mmol) of sodium-tert-butoxide, 20 mL of xylene, 11 mg (47 μmol) of palladium acetate and 76 mg (94 μmol) of a 25% by weight xylene solution of tri(tert-butyl)phosphine were added to a 100-mL three-neck flask and were stirred at 140° C. for 22 hours. After cooling to room temperature, 22 mL of pure water was added thereto and stirred. An aqueous layer and an organic layer were then separated, and the organic layer was washed with saturated aqueous sodium chloride. The organic layer was dried over anhydrous magnesium sulfate and was then subjected to column chromatography with a small amount of silica gel to remove a polar component. The solvent was then distilled off under reduced pressure, and the resulting solid was recrystallized with a mixed solvent of toluene and butanol to isolate 3.0 g (3.4 mmol) of a compound (E264) as a white solid (yield: 73%). E264 had a sublimation temperature of 340° C., and it was confirmed that the sublimate E264 was glassy.

The compound was identified by FDMS measurement.

FDMS: 868

### Example 38 (Synthesis of Compound (E268))

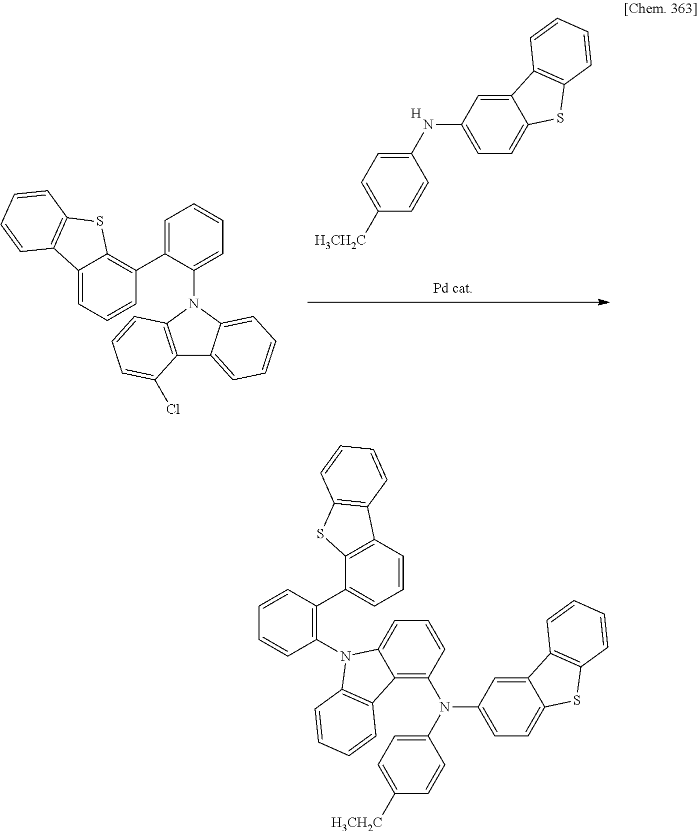

In a nitrogen stream, 3.7 g (8.0 mmol) of 4-chloro-9-(2-(dibenzo[b,d]thiophen-4-yl)phenyl)-9H-carbazole prepared in Synthesis Example 34, 2.3 g (7.6 mmol) of N-(4-ethylphenyl)dibenzo[b,d]thiophene-2-amine, 0.95 g (9.9 mmol) of sodium-tert-butoxide, 20 mL of xylene, 17 mg (76 μmol) of palladium acetate and 0.12 g (0.15 mmol) of a 25% by weight xylene solution of tri(tert-butyl)phosphine were added to a 100-mL three-neck flask and were stirred at 140° C. for 22 hours. After cooling to room temperature, 22 mL of pure water was added thereto and stirred. An aqueous layer and an organic layer were then separated, and the organic layer was washed with saturated aqueous sodium chloride. The organic layer was dried over anhydrous magnesium sulfate and was then subjected to column chromatography with a small amount of silica gel to remove a polar component. The solvent was then distilled off under reduced pressure, and the resulting solid was recrystallized with a mixed solvent of toluene and butanol to isolate 4.8 g (6.6 mmol) of a compound (E268) as a white solid (yield: 87%). E268 had a sublimation temperature of 300° C., and it was confirmed that the sublimate E268 was glassy.

The compound was identified by FDMS measurement.

FDMS: 726

### Example 39 (Synthesis of Compound (E303))

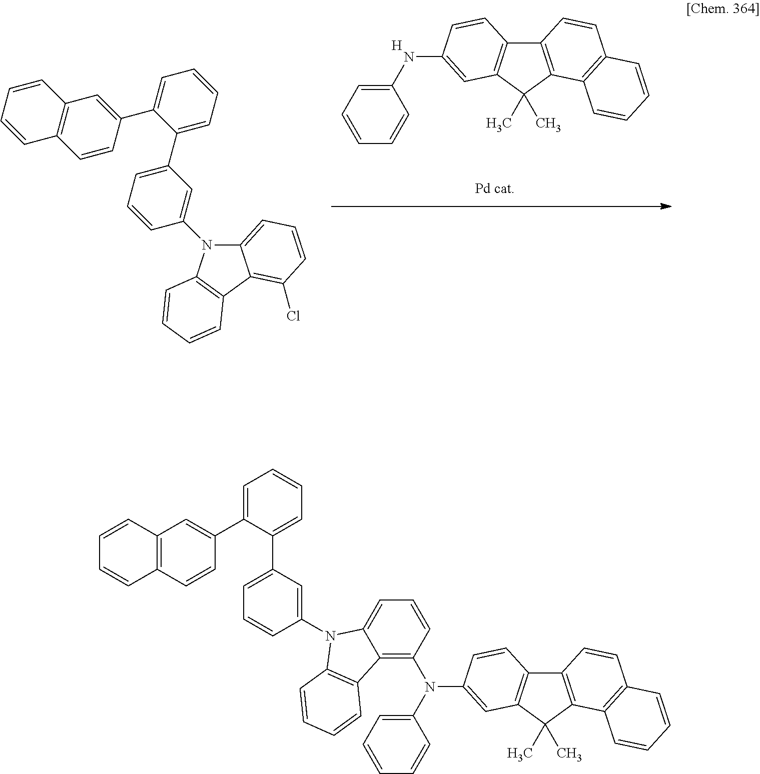

In a nitrogen stream, 4.7 g (9.7 mmol) of 4-chloro-9-(2′-(naphthalen-2-yl)-[1,1′-biphenyl]-3-yl)-9H-carbazole, 3.1 g (9.2 mmol) of 11,11-dimethyl-N-phenyl-11H-benzo[a]fluorene-9-amine, 1.2 g (12 mmol) of sodium-tert-butoxide, 20 mL of xylene, 21 mg (92 μmol) of palladium acetate and 0.15 g (0.18 mmol) of a 25% by weight xylene solution of tri(tert-butyl)phosphine were added to a 100-mL three-neck flask and were stirred at 140° C. for 22 hours. After cooling to room temperature, 22 mL of pure water was added thereto and stirred. An aqueous layer and an organic layer were then separated, and the organic layer was washed with saturated aqueous sodium chloride. The organic layer was dried over anhydrous magnesium sulfate and was then subjected to column chromatography with a small amount of silica gel to remove a polar component. The solvent was then distilled off under reduced pressure, and the resulting solid was recrystallized with a mixed solvent of toluene and butanol to isolate 5.0 g (6.5 mmol) of a compound (E303) as a white solid (yield: 70%). E303 had a sublimation temperature of 320° C., and it was confirmed that the sublimate E303 was glassy.

The compound was identified by FDMS measurement.

FDMS: 778

### Example 40 (Synthesis of Compound (E329))

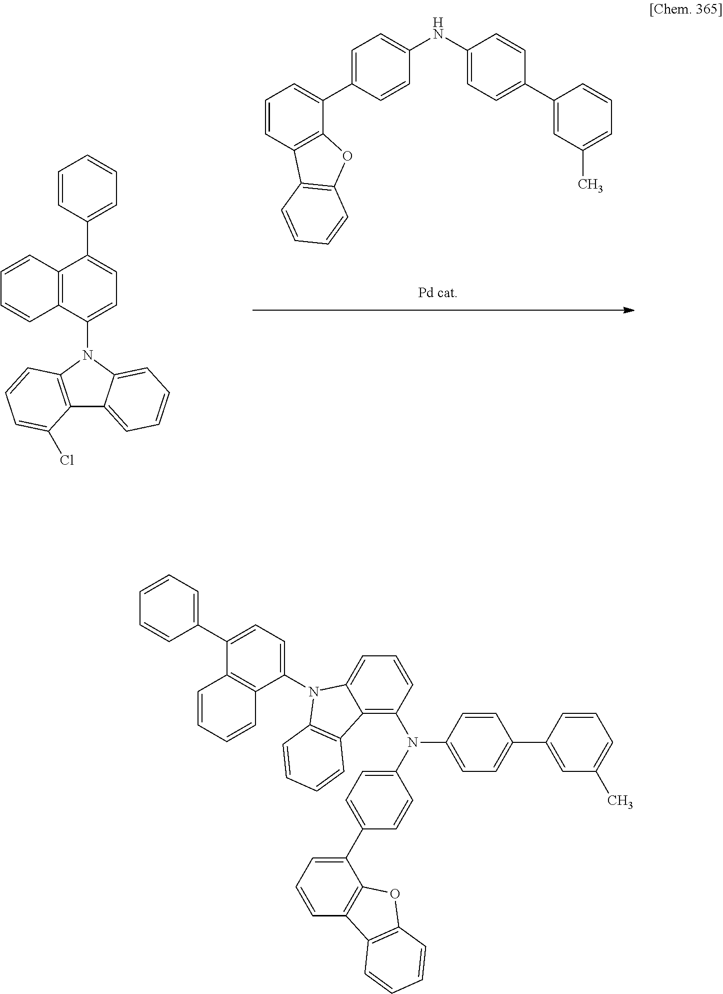 [SMILES: Clc1cccc2c1c1ccccc1n2-c1ccc(-c2ccccc2)c2ccccc12 ; Cc1cccc(-c2ccc(Nc3ccc(-c4cccc5c4oc4ccccc45)cc3)cc2)c1 ; Cc1cccc(-c2ccc(N(c3ccc(-c4cccc5c4oc4ccccc45)cc3)c3cccc4c3c3ccccc3n4-c3ccc(-c4ccccc4)c4ccccc34)cc2)c1]

In a nitrogen stream, 2.5 g (6.2 mmol) of 4-chloro-9-(4-phenylnaphthalen-1-yl)-9H-carbazole prepared in Synthesis Example 36, 2.5 g (5.9 mmol) of N-(4-(dibenzo[b,d]furan-4-yl)phenyl)-3′-methyl-[1,1′-biphenyl]-4-amine, 0.73 g (7.6 mmol) of sodium-tert-butoxide, 20 mL of xylene, 13 mg (59 μmol) of palladium acetate and 95 mg (0.12 mmol) of a 25% by weight xylene solution of tri(tert-butyl)phosphine were added to a 100-mL three-neck flask and were stirred at 140° C. for 22 hours. After cooling to room temperature, 22 mL of pure water was added thereto and stirred. An aqueous layer and an organic layer were then separated, and the organic layer was washed with saturated aqueous sodium chloride. The organic layer was dried over anhydrous magnesium sulfate and was then subjected to column chromatography with a small amount of silica gel to remove a polar component. The solvent was then distilled off under reduced pressure, and the resulting solid was recrystallized with a mixed solvent of toluene and butanol to isolate 3.7 g (4.7 mmol) of a compound (E329) as a white solid (yield: 80%). E329 had a sublimation temperature of 335° C., and it was confirmed that the sublimate E329 was glassy.

The compound was identified by FDMS measurement.

FDMS: 792

### Example 41 (Synthesis of Compound (E330))

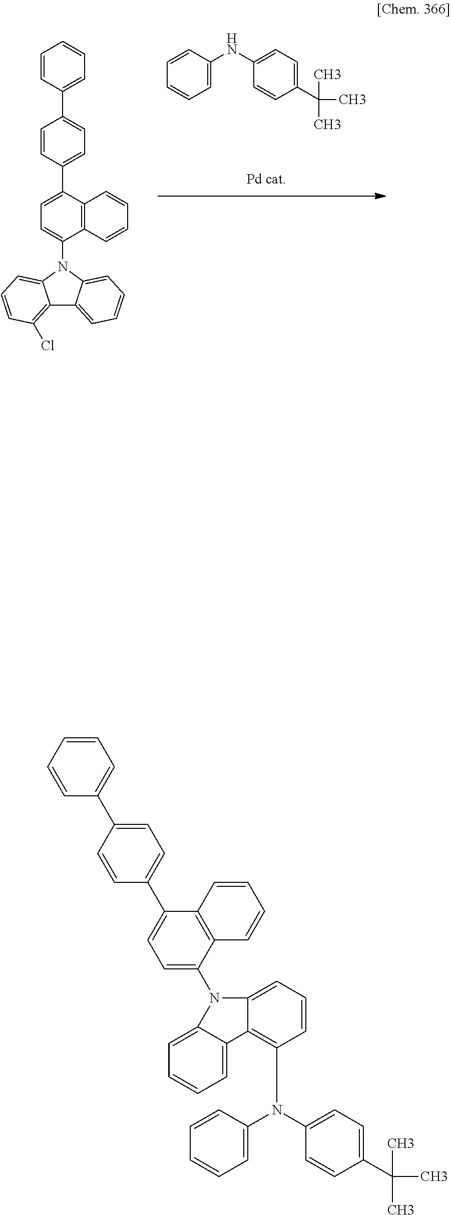 [SMILES: Clc1cccc2c1c1ccccc1n2-c1ccc(-c2ccc(-c3ccccc3)cc2)c2ccccc12 ; CC(C)(C)c1ccc(Nc2ccccc2)cc1 ; CC(C)(C)c1ccc(N(c2ccccc2)c2cccc3c2c2ccccc2n3-c2ccc(-c3ccc(-c4ccccc4)cc3)c3ccccc23)cc1]

In a nitrogen stream, 4.5 g (9.3 mmol) of 9-(4-([1,1′-biphenyl]-4-yl)naphthalen-1-yl)-4-chloro-9H-carbazole prepared in Synthesis Example 37, 2.0 g (8.9 mmol) of 4-(tert-butyl)-N-phenylaniline, 1.1 g (12 mmol) of sodium-tert-butoxide, 20 mL of xylene, 20 mg (89 μmol) of palladium acetate and 0.14 g (0.18 mmol) of a 25% by weight xylene solution of tri(tert-butyl)phosphine were added to a 100-mL three-neck flask and were stirred at 140° C. for 22 hours. After cooling to room temperature, 22 mL of pure water was added thereto and stirred. An aqueous layer and an organic layer were then separated, and the organic layer was washed with saturated aqueous sodium chloride. The organic layer was dried over anhydrous magnesium sulfate and was then subjected to column chromatography with a small amount of silica gel to remove a polar component. The solvent was then distilled off under reduced pressure, and the resulting solid was recrystallized with a mixed solvent of toluene and butanol to isolate 5.2 g (7.8 mmol) of a compound (E330) as a white solid (yield: 88%). E330 had a sublimation temperature of 295° C., and it was confirmed that the sublimate E330 was glassy.

The compound was identified by FDMS measurement.

FDMS: 423

### Example 42 (Synthesis of Compound (E341))

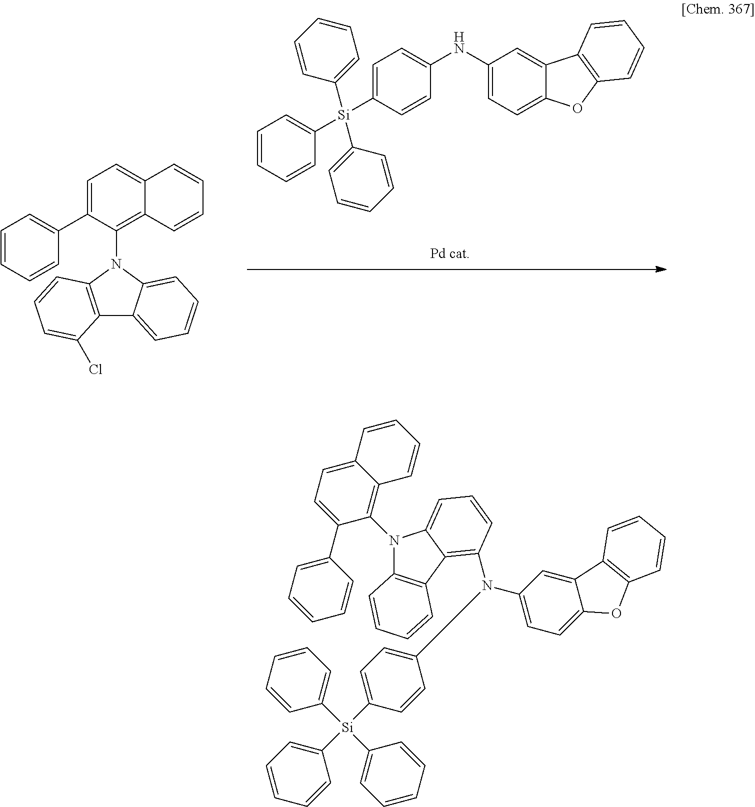 [SMILES: Clc1cccc2c1c1ccccc1n2-c1c(-c2ccccc2)ccc2ccccc12 ; c1ccc([Si](c2ccccc2)(c2ccccc2)c2ccc(Nc3ccc4oc5ccccc5c4c3)cc2)cc1 ; c1ccc(-c2ccc3ccccc3c2-n2c3ccccc3c3c(N(CCc4ccc([Si](c5ccccc5)(c5ccccc5)c5ccccc5)cc4)c4ccc5oc6ccccc6c5c4)cccc32)cc1]

In a nitrogen stream, 1.3 g (3.2 mmol) of 4-chloro-9-(2-phenylnaphthalen-1-yl)-9H-carbazole prepared in Synthesis Example 38, 1.6 g (3.1 mmol) of N-(4-(triphenylsilyl)phenyl)dibenzo[b,d]furan-2-amine, 0.39 g (4.0 mmol) of sodium-tert-butoxide, 20 mL of xylene, 6.9 mg (31 μmol) of palladium acetate and 50 mg (62 μmol) of a 25% by weight xylene solution of tri(tert-butyl)phosphine were added to a 100-mL three-neck flask and were stirred at 140° C. for 22 hours. After cooling to room temperature, 22 mL of pure water was added thereto and stirred. An aqueous layer and an organic layer were then separated, and the organic layer was washed with saturated aqueous sodium chloride. The organic layer was dried over anhydrous magnesium sulfate and was then subjected to column chromatography with a small amount of silica gel to remove a polar component. The solvent was then distilled off under reduced pressure, and the resulting solid was recrystallized with a mixed solvent of toluene and butanol to isolate 1.8 g (2.0 mmol) of a compound (E341) as a white solid (yield: 65%). E341 had a sublimation temperature of 345° C., and it was confirmed that the sublimate E341 was glassy.

The compound was identified by FDMS measurement.

FDMS: 884

### Example 43 (Synthesis of Compound (E674))

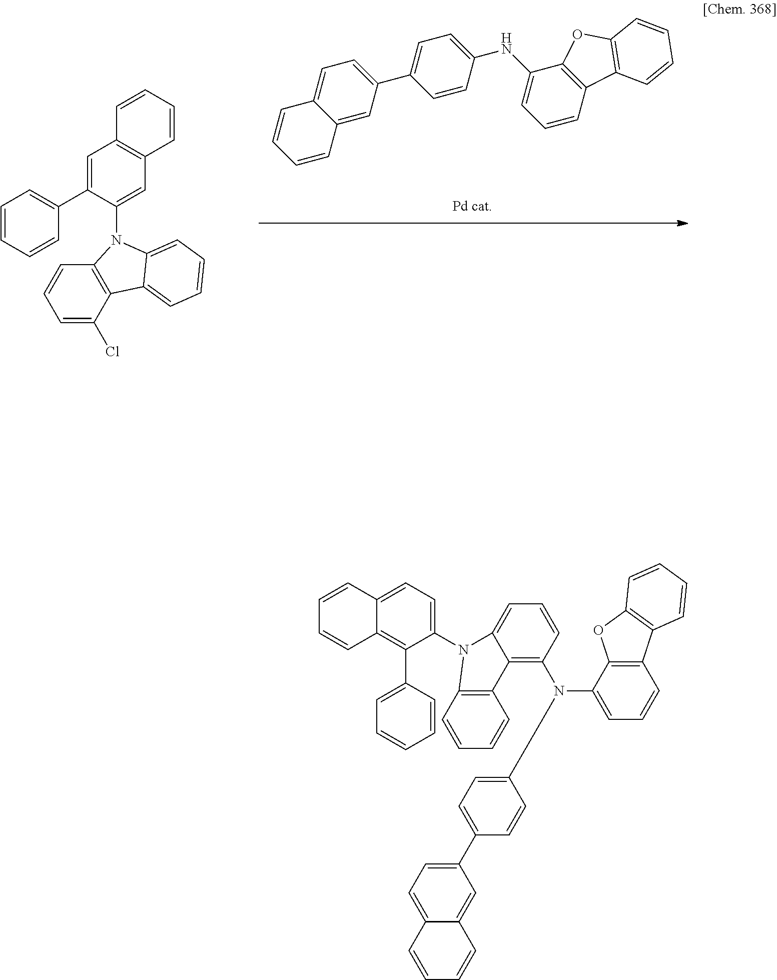 [SMILES: Clc1cccc2c1c1ccccc1n2-c1cc2ccccc2cc1-c1ccccc1 ; c1ccc2cc(-c3ccc(Nc4cccc5c4oc4ccccc45)cc3)ccc2c1 ; c1ccc(-c2c(-n3c4ccccc4c4c(N(CCc5ccc(-c6ccc7ccccc7c6)cc5)c5cccc6c5oc5ccccc56)cccc43)ccc3ccccc23)cc1]

In a nitrogen stream, 3.6 g (9.0 mmol) of 4-chloro-9-(3-phenylnaphthalen-2-yl)-9H-carbazole prepared in Synthesis Example 40, 3.3 g (8.6 mmol) of N-(4-(naphthalen-2-yl)phenyl)dibenzo[b,d]furan-4-amine, 1.1 g (11 mmol) of sodium-tert-butoxide, 20 mL of xylene, 19 mg (86 μmol) of palladium acetate and 0.14 g (0.17 mmol) of a 25% by weight xylene solution of tri(tert-butyl)phosphine were added to a 100-mL three-neck flask and were stirred at 140° C. for 22 hours. After cooling to room temperature, 22 mL of pure water was added thereto and stirred. An aqueous layer and an organic layer were then separated, and the organic layer was washed with saturated aqueous sodium chloride. The organic layer was dried over anhydrous magnesium sulfate and was then subjected to column chromatography with a small amount of silica gel to remove a polar component. The solvent was then distilled off under reduced pressure, and the resulting solid was recrystallized with a mixed solvent of toluene and butanol to isolate 4.9 g (6.5 mmol) of a compound (E674) as a white solid (yield: 76%). E674 had a sublimation temperature of 320° C., and it was confirmed that the sublimate E674 was glassy.

The compound was identified by FDMS measurement.

FDMS: 752

### Example 44 (Synthesis of Compound (E770))

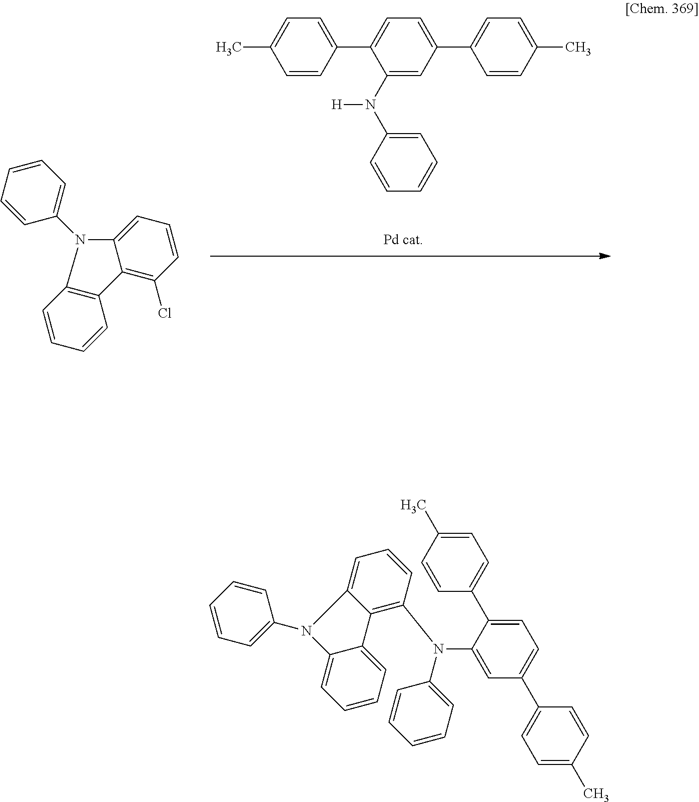

In a nitrogen stream, 1.6 g (5.7 mmol) of 4-chloro-9-phenyl-9H-carbazole, 1.9 g (5.4 mmol) of 4,4″-dimethyl-N-phenyl-[1,1′:4′,1″-terphenyl]-2′-amine, 0.68 g (7.1 mmol) of sodium-tert-butoxide, 20 mL of xylene, 12 mg (54 μmol) of palladium acetate and 88 mg (0.11 mmol) of a 25% by weight xylene solution of tri(tert-butyl)phosphine were added to a 100-mL three-neck flask and were stirred at 140° C. for 22 hours. After cooling to room temperature, 22 mL of pure water was added thereto and stirred. An aqueous layer and an organic layer were then separated, and the organic layer was washed with saturated aqueous sodium chloride. The organic layer was dried over anhydrous magnesium sulfate and was then subjected to column chromatography with a small amount of silica gel to remove a polar component. The solvent was then distilled off under reduced pressure, and the resulting solid was recrystallized with a mixed solvent of toluene and butanol to isolate 2.5 g (4.2 mmol) of a compound (E770) as a white solid (yield: 77%). E770 had a sublimation temperature of 260° C., and it was confirmed that the sublimate E770 was glassy.

The compound was identified by FDMS measurement.

FDMS: 590

### Example 45 (Synthesis of Compound (F166))

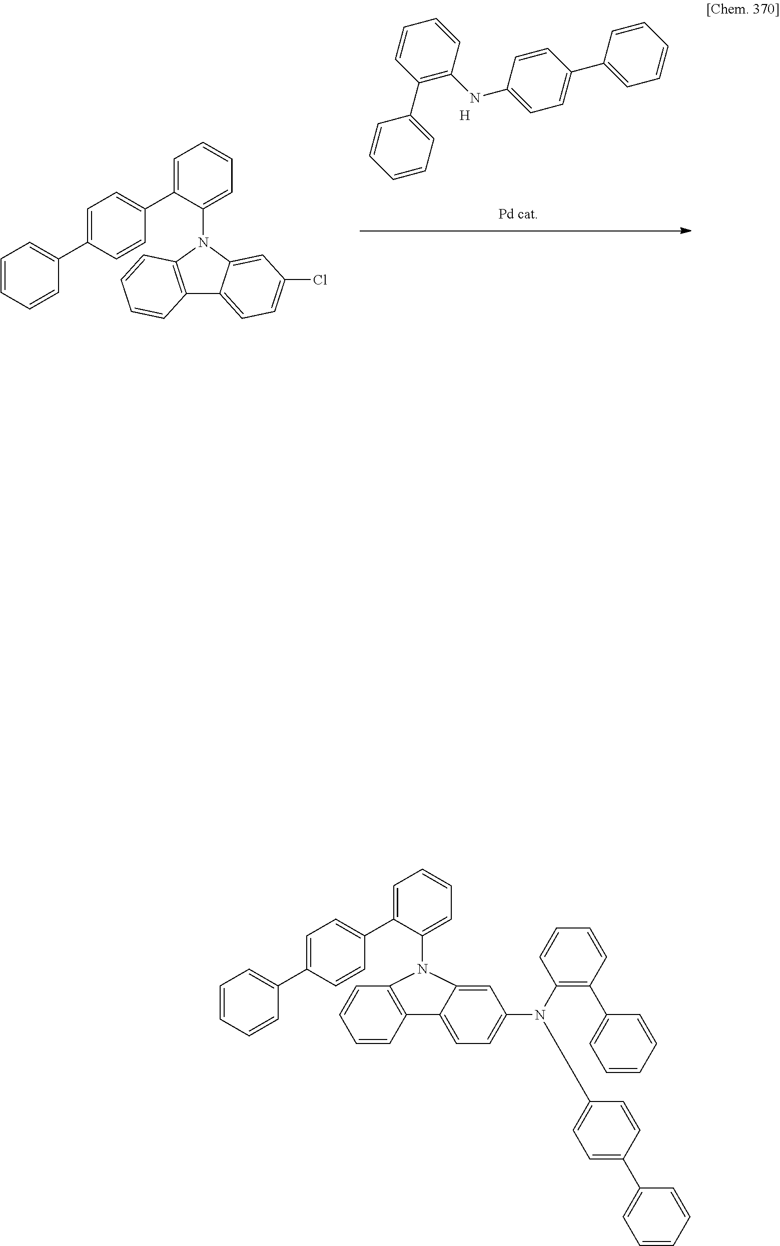

In a nitrogen stream, 4.2 g (9.8 mmol) of 9-([1,1′:4,1″-terphenyl]-2-yl)-2-chloro-9H-carbazole prepared in Synthesis Example 8, 3.0 g (9.3 mmol) of N-([1,1′-biphenyl]-4-yl)-[1,1′-biphenyl]-2-amine, 1.2 g (12 mmol) of sodium-tert-butoxide, 20 mL of xylene, 21 mg (93 μmol) of palladium acetate and 0.15 g (0.19 mmol) of a 25% by weight xylene solution of tri(tert-butyl)phosphine were added to a 100-mL three-neck flask and were stirred at 140° C. for 22 hours. After cooling to room temperature, 22 mL of pure water was added thereto and stirred. An aqueous layer and an organic layer were then separated, and the organic layer was washed with saturated aqueous sodium chloride. The organic layer was dried over anhydrous magnesium sulfate and was then subjected to column chromatography with a small amount of silica gel to remove a polar component. The solvent was then distilled off under reduced pressure, and the resulting solid was recrystallized with a mixed solvent of toluene and butanol to isolate 5.3 g (7.5 mmol) of a compound (F166) as a white solid (yield: 80%). F166 had a sublimation temperature of 285° C., and it was confirmed that the sublimate F166 was glassy.

The compound was identified by FDMS measurement.

FDMS: 714

### Example 46 (Synthesis of Compound (F228))

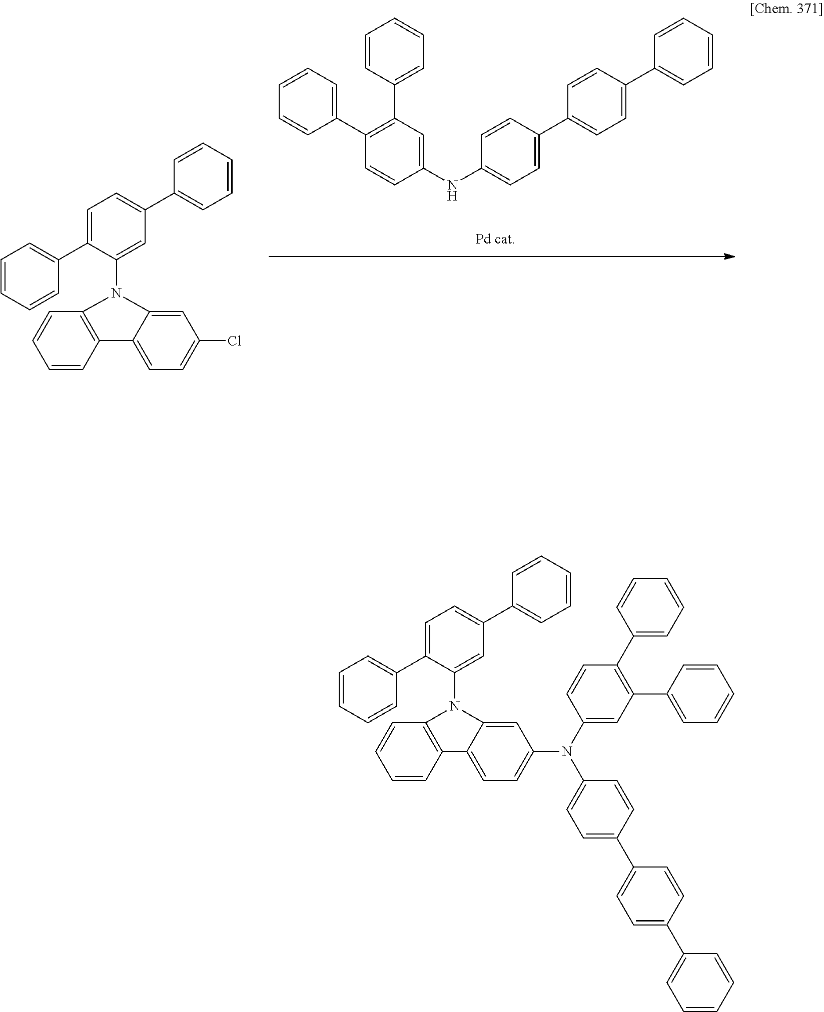 [SMILES: Clc1ccc2c3ccccc3n(-c3cc(-c4ccccc4)ccc3-c3ccccc3)c2c1 ; c1ccc(-c2ccc(-c3ccc(Nc4ccc(-c5ccccc5)c(-c5ccccc5)c4)cc3)cc2)cc1 ; c1ccc(-c2ccc(-c3ccc(N(c4ccc(-c5ccccc5)c(-c5ccccc5)c4)c4ccc5c6ccccc6n(-c6cc(-c7ccccc7)ccc6-c6ccccc6)c5c4)cc3)cc2)cc1]

In a nitrogen stream, 1.8 g (4.2 mmol) of 9-([1,1:4′,1″-terphenyl]-2′-yl)-2-chloro-9H-carbazole prepared in Synthesis Example 4, 1.9 g (4.0 mmol) of N-([1,1′:4′,1″-terphenyl]-4-yl)-[1,1′:2′,1″-terphenyl]-4′-amine, 0.50 g (5.2 mmol) of sodium-tert-butoxide, 20 mL of xylene, 9.0 mg (40 μmol) of palladium acetate and 65 mg (80 μmol) of a 25% by weight xylene solution of tri(tert-butyl)phosphine were added to a 100-mL three-neck flask and were stirred at 140° C. for 22 hours. After cooling to room temperature, 22 mL of pure water was added thereto and stirred. An aqueous layer and an organic layer were then separated, and the organic layer was washed with saturated aqueous sodium chloride. The organic layer was dried over anhydrous magnesium sulfate and was then subjected to column chromatography with a small amount of silica gel to remove a polar component. The solvent was then distilled off under reduced pressure, and the resulting solid was recrystallized with a mixed solvent of toluene and butanol to isolate 2.9 g (3.3 mmol) of a compound (F228) as a white solid (yield: 83%). F228 had a sublimation temperature of 330° C., and it was confirmed that the sublimate F228 was glassy.

The compound was identified by FDMS measurement.

FDMS: 866

### Example 47 (Synthesis of Compound (F320))

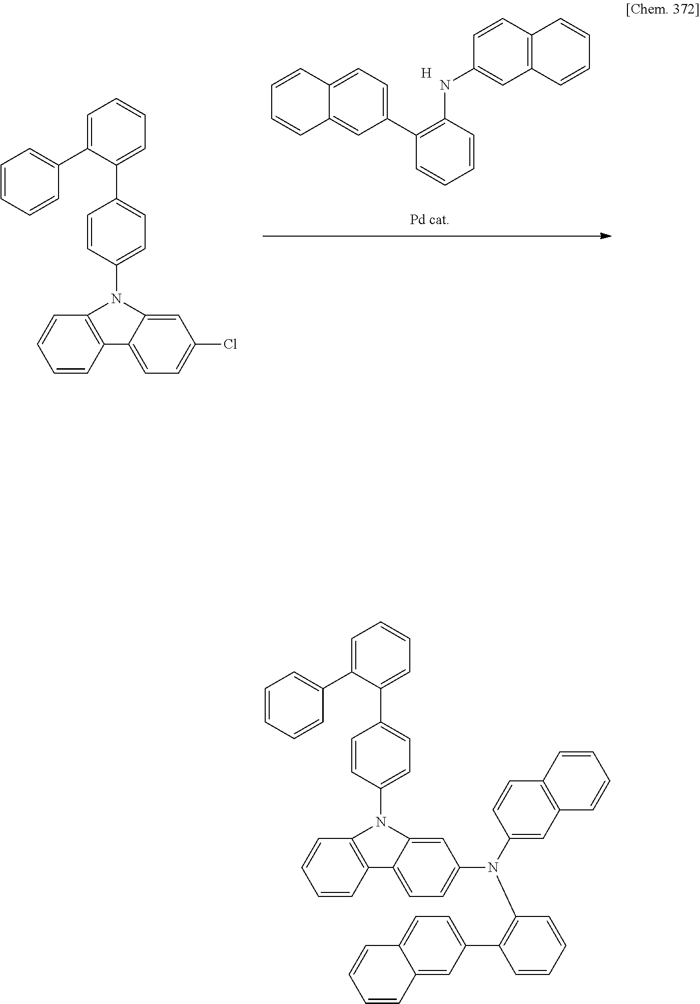

In a nitrogen stream, 3.4 g (7.9 mmol) of 9-([1,1′:2′,1″-terphenyl]-4-yl)-2-chloro-9H-carbazole, 2.6 g (7.5 mmol) of N-(2-(naphthalen-2-yl)phenyl)naphthalene-2-amine, 0.94 g (9.8 mmol) of sodium-tert-butoxide, 20 mL of xylene, 17 mg (75 μmol) of palladium acetate and 0.12 g (0.15 mmol) of a 25% by weight xylene solution of tri(tert-butyl)phosphine were added to a 100-mL three-neck flask and were stirred at 140° C. for 22 hours. After cooling to room temperature, 22 mL of pure water was added thereto and stirred. An aqueous layer and an organic layer were then separated, and the organic layer was washed with saturated aqueous sodium chloride. The organic layer was dried over anhydrous magnesium sulfate and was then subjected to column chromatography with a small amount of silica gel to remove a polar component. The solvent was then distilled off under reduced pressure, and the resulting solid was recrystallized with a mixed solvent of toluene and butanol to isolate 3.8 g (5.2 mmol) of a compound (F320) as a white solid (yield: 69%). F320 had a sublimation temperature of 300° C., and it was confirmed that the sublimate F320 was glassy.

The compound was identified by FDMS measurement.

FDMS: 738

### Example 48 (Synthesis of Compound (F424))

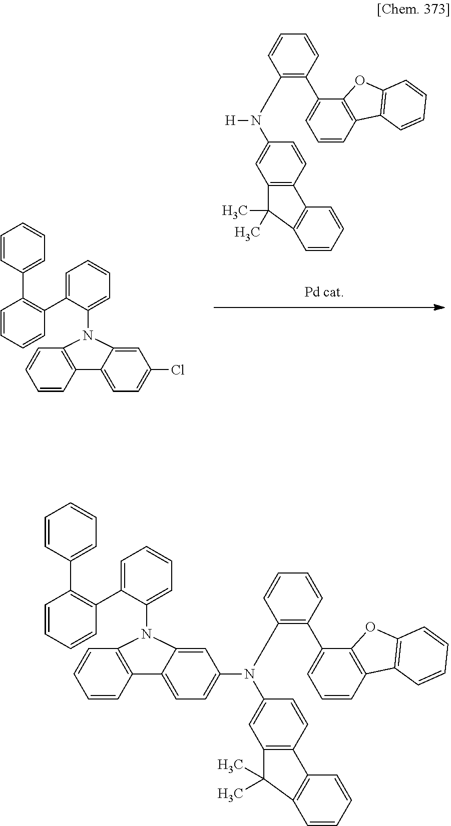 [SMILES: Clc1ccc2c3ccccc3n(-c3ccccc3-c3ccccc3-c3ccccc3)c2c1 ; CC1(C)c2ccccc2-c2ccc(NCc3ccccc3-c3cccc4c3oc3ccccc34)cc21 ; CC1(C)c2ccccc2-c2ccc(N(Cc3ccccc3-c3cccc4c3oc3ccccc34)c3ccc4c5ccccc5n(-c5ccccc5-c5ccccc5-c5ccccc5)c4c3)cc21]

In a nitrogen stream, 3.0 g (7.0 mmol) of 9-([1,1′:2′,1″-terphenyl]-2-yl)-2-chloro-9H-carbazole, 3.0 g (6.6 mmol) of N-(2-(dibenzo[b,d]furan-4-yl)phenyl)-9,9-dimethyl-9H-fluorene-2-amine, 0.83 g (8.6 mmol) of sodium-tert-butoxide, 20 mL of xylene, 15 mg (66 μmol) of palladium acetate and 0.11 g (0.13 mmol) of a 25% by weight xylene solution of tri(tert-butyl)phosphine were added to a 100-mL three-neck flask and were stirred at 140° C. for 22 hours. After cooling to room temperature, 22 mL of pure water was added thereto and stirred. An aqueous layer and an organic layer were then separated, and the organic layer was washed with saturated aqueous sodium chloride. The organic layer was dried over anhydrous magnesium sulfate and was then subjected to column chromatography with a small amount of silica gel to remove a polar component. The solvent was then distilled off under reduced pressure, and the resulting solid was recrystallized with a mixed solvent of toluene and butanol to isolate 4.5 g (5.3 mmol) of a compound (F424) as a white solid (yield: 80%). F424 had a sublimation temperature of 325° C., and it was confirmed that the sublimate F424 was glassy.

The compound was identified by FDMS measurement.

FDMS: 844

### Example 49 (Synthesis of Compound (F839))

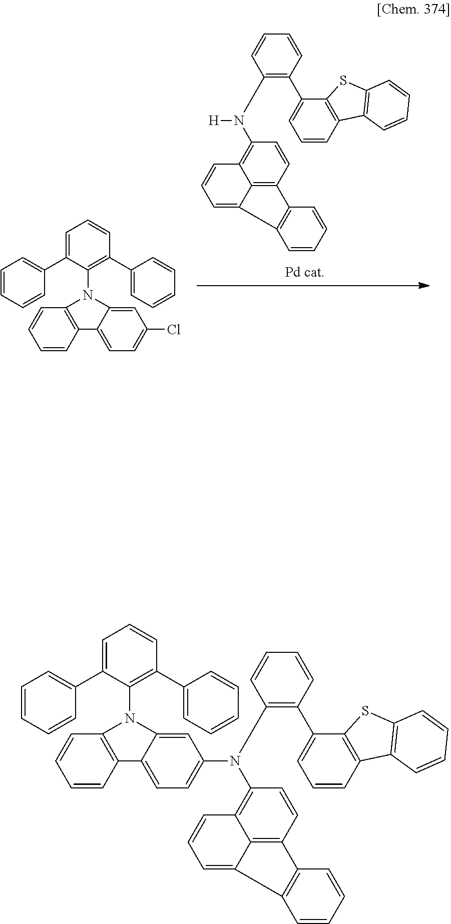 [SMILES: Clc1ccc2c3ccccc3n(-c3c(-c4ccccc4)cccc3-c3ccccc3)c2c1 ; c1ccc(-c2cccc3c2sc2ccccc23)c(CNc2ccc3c4c(cccc24)-c2ccccc2-3)c1 ; c1ccc(-c2cccc(-c3ccccc3)c2-n2c3ccccc3c3ccc(N(Cc4ccccc4-c4cccc5c4sc4ccccc45)c4ccc5c6c(cccc46)-c4ccccc4-5)cc32)cc1]

In a nitrogen stream, 2.4 g (5.5 mmol) of 9-([1,1′:3′,1″-terphenyl]-2′-yl)-2-chloro-9H-carbazole prepared in Synthesis Example 5, 2.5 g (5.3 mmol) of N-(2-(dibenzo[b,d]thiophen-4-yl)phenyl)fluoranthene-3-amine, 0.66 g (6.8 mmol) of sodium-tert-butoxide, 20 mL of xylene, 12 mg (53 μmol) of palladium acetate and 85 mg (0.11 mmol) of a 25% by weight xylene solution of tri(tert-butyl)phosphine were added to a 100-mL three-neck flask and were stirred at 140° C. for 22 hours. After cooling to room temperature, 22 mL of pure water was added thereto and stirred. An aqueous layer and an organic layer were then separated, and the organic layer was washed with saturated aqueous sodium chloride. The organic layer was dried over anhydrous magnesium sulfate and was then subjected to column chromatography with a small amount of silica gel to remove a polar component. The solvent was then distilled off under reduced pressure, and the resulting solid was recrystallized with a mixed solvent of toluene and butanol to isolate 2.3 g (2.7 mmol) of a compound (F839) as a white solid (yield: 51%). F839 had a sublimation temperature of 315° C., and it was confirmed that the sublimate F839 was glassy.

The compound was identified by FDMS measurement.

FDMS: 868

### Example 50 (Synthesis of Compound (F901))

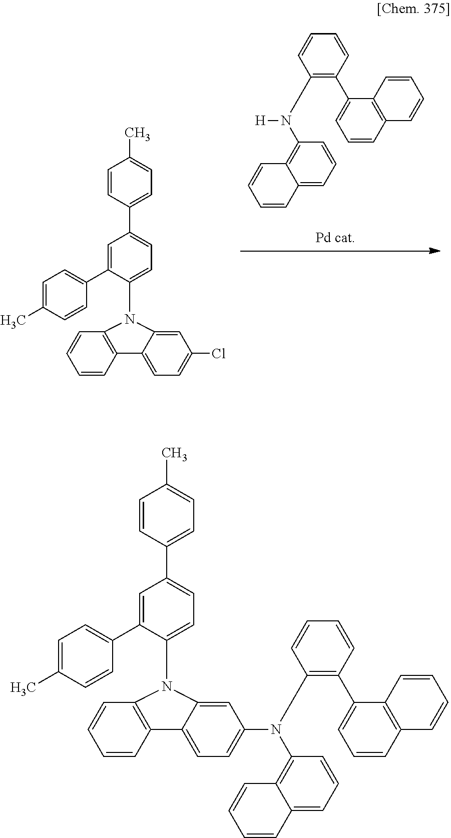 [SMILES: Cc1ccc(-c2ccc(-n3c4ccccc4c4ccc(Cl)cc43)c(-c3ccc(C)cc3)c2)cc1 ; c1ccc(-c2cccc3ccccc23)c(CNc2cccc3ccccc23)c1 ; Cc1ccc(-c2ccc(-n3c4ccccc4c4ccc(N(Cc5ccccc5-c5cccc6ccccc56)c5cccc6ccccc56)cc43)c(-c3ccc(C)cc3)c2)cc1]

In a nitrogen stream, 3.6 g (7.9 mmol) of 2-chloro-9-(4,4″-dimethyl-[1,1′:3′,1″-terphenyl]-4′-yl)-9H-carbazole prepared in Synthesis Example 39, 2.6 g (7.5 mmol) of N-(2-(naphthalen-1-yl)phenyl)naphthalene-1-amine, 0.94 g (9.8 mmol) of sodium-tert-butoxide, 20 mL of xylene, 17 mg (75 μmol) of palladium acetate and 0.12 g (0.15 mmol) of a 25% by weight xylene solution of tri(tert-butyl)phosphine were added to a 100-mL three-neck flask and were stirred at 140° C. for 22 hours. After cooling to room temperature, 22 mL of pure water was added thereto and stirred. An aqueous layer and an organic layer were then separated, and the organic layer was washed with saturated aqueous sodium chloride. The organic layer was dried over anhydrous magnesium sulfate and was then subjected to column chromatography with a small amount of silica gel to remove a polar component. The solvent was then distilled off under reduced pressure, and the resulting solid was recrystallized with a mixed solvent of toluene and butanol to isolate 4.6 g (5.9 mmol) of a compound (F901) as a white solid (yield: 79%). F901 had a sublimation temperature of 290° C., and it was confirmed that the sublimate F901 was glassy.

The compound was identified by FDMS measurement.

FDMS: 766

### Example 51 (Synthesis of Compound (G360))

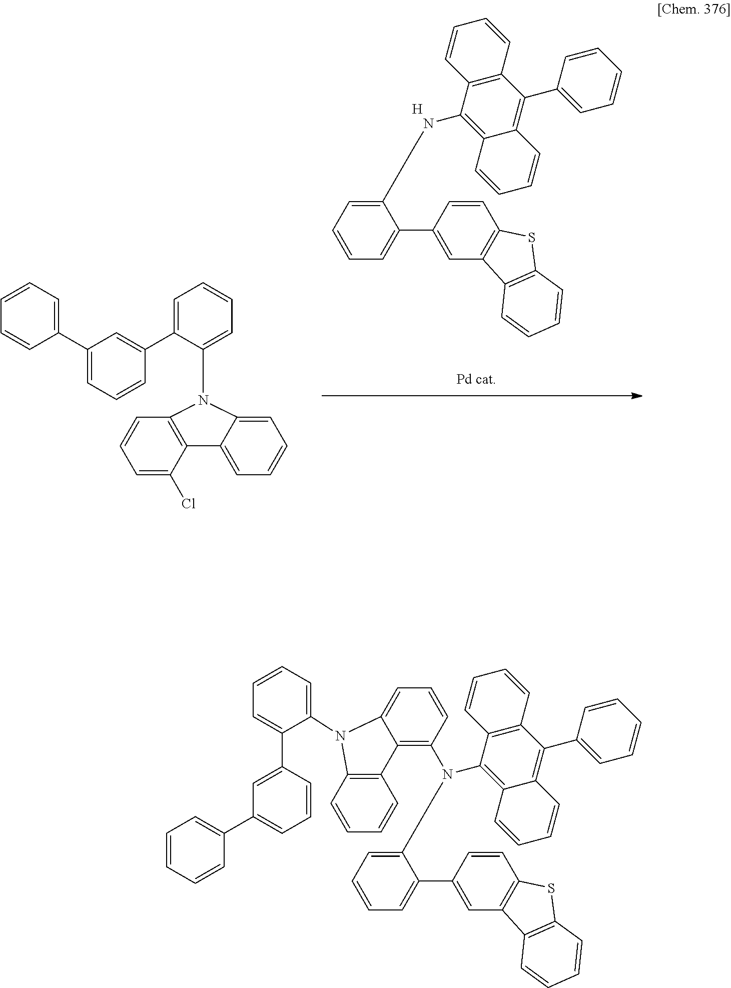

In a nitrogen stream, 2.0 g (4.6 mmol) of 9-([1,1′:3′,1″-terphenyl]-2-yl)-4-chloro-9H-carbazole prepared in Synthesis Example 41, 2.2 g (4.2 mmol) of N-(2-(dibenzo[b,d]thiophen-2-yl)phenyl)-10-phenylanthracene-9-amine, 0.52 g (5.4 mmol) of sodium-tert-butoxide, 20 mL of xylene, 9.4 mg (42 μmol) of palladium acetate and 67 mg (83 μmol) of a 25% by weight xylene solution of tri(tert-butyl)phosphine were added to a 100-mL three-neck flask and were stirred at 140° C. for 22 hours. After cooling to room temperature, 22 mL of pure water was added thereto and stirred. An aqueous layer and an organic layer were then separated, and the organic layer was washed with saturated aqueous sodium chloride. The organic layer was dried over anhydrous magnesium sulfate and was then subjected to column chromatography with a small amount of silica gel to remove a polar component. The solvent was then distilled off under reduced pressure, and the resulting solid was recrystallized with a mixed solvent of toluene and butanol to isolate 2.6 g (2.8 mmol) of a compound (G360) as a white solid (yield: 68%). G360 had a sublimation temperature of 350° C., and it was confirmed that the sublimate G360 was glassy.

The compound was identified by FDMS measurement.

FDMS: 920

### Example 52 (Synthesis of Compound (G702))

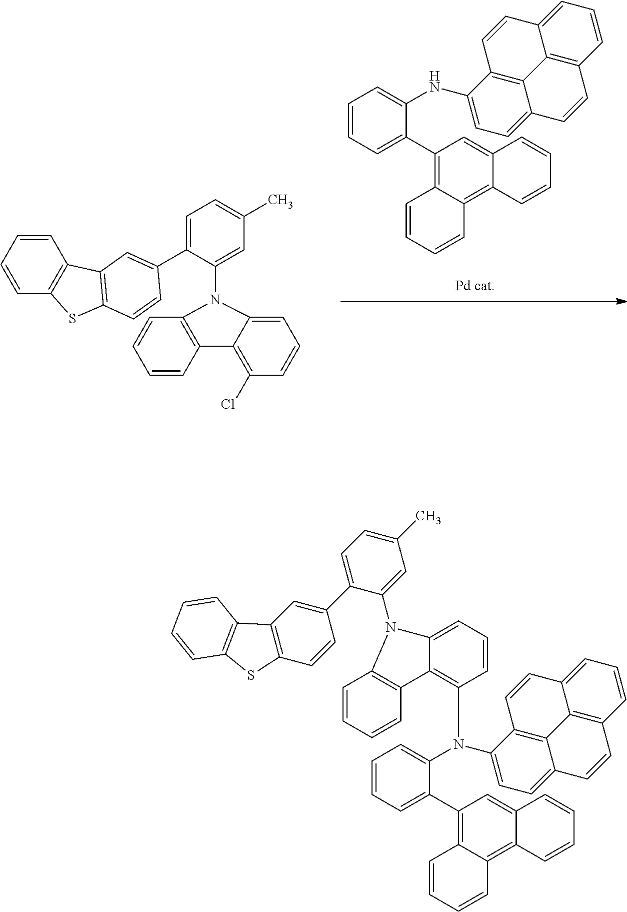 [SMILES: Cc1ccc(-c2ccc3sc4ccccc4c3c2)c(-n2c3ccccc3c3c(Cl)cccc32)c1 ; c1ccc(-c2cc3ccccc3c3ccccc23)c(Nc2ccc3ccc4cccc5ccc2c3c45)c1 ; Cc1ccc(-c2ccc3sc4ccccc4c3c2)c(-n2c3ccccc3c3c(N(c4ccccc4-c4cc5ccccc5c5ccccc45)c4ccc5ccc6cccc7ccc4c5c67)cccc32)c1]

In a nitrogen stream, 1.9 g (4.0 mmol) of 4-chloro-9-(2-(dibenzo[b,d]thiophen-2-yl)-5-methylphenyl)-9H-carbazole prepared in Synthesis Example 42, 1.8 g (3.8 mmol) of N-(2-(phenanthren-9-yl)phenyl)pyrene-2-amine, 0.48 g (5.0 mmol) of sodium-tert-butoxide, 20 mL of xylene, 8.6 mg (38 μmol) of palladium acetate and 62 mg (77 μmol) of a 25% by weight xylene solution of tri(tert-butyl)phosphine were added to a 100-mL three-neck flask and were stirred at 140° C. for 22 hours. After cooling to room temperature, 22 mL of pure water was added thereto and stirred. An aqueous layer and an organic layer were then separated, and the organic layer was washed with saturated aqueous sodium chloride. The organic layer was dried over anhydrous magnesium sulfate and was then subjected to column chromatography with a small amount of silica gel to remove a polar component. The solvent was then distilled off under reduced pressure, and the resulting solid was recrystallized with a mixed solvent of toluene and butanol to isolate 1.6 g (1.7 mmol) of a compound (G702) as a white solid (yield: 45%). G702 had a sublimation temperature of 350° C., and it was confirmed that the sublimate G702 was glassy.

The compound was identified by FDMS measurement.

FDMS: 906

### Examples of Transverse Current Measuring Elements

### Example 53 (Evaluation of Transverse Current of Compound D68))

A glass substrate on which an interdigitated ITO electrode with a thickness of 160 nm is formed is used to measure the transverse current. Two interdigitated ITO electrodes each with an electrode width of 20 μm and a length of 2 mm are formed on the glass substrate. The gap between the two interdigitated electrodes is set to be 80 μm.

The glass substrate was subjected to ultrasonic cleaning with ultrapure water. Surface treatment was performed by ozone ultraviolet cleaning. The glass substrate was introduced into a vacuum evaporation bath, and the pressure was reduced to 1.0×10⁻⁴ Pa with a vacuum pump. Each layer was then formed in the following order under their respective film-forming conditions. Each organic material was formed into a film by a resistance heating method.

### (Preparation of Hole-Injection Layer)

The compound (D68), which is a transverse current suppressing material purified by sublimation in Example 1, and 1,2,3-tris[(4-cyano-2,3,5,6-tetrafluorophenyl)methylene]cyclopropane were formed into a film with a thickness of 10 nm at a ratio of 99:1 (mass ratio) to prepare a hole-injection layer.

### (Preparation of Hole-Transport Layer)

The compound (D68), which is a transverse current suppressing material purified by sublimation in Example 1, was formed into a film with a thickness of 100 nm at a rate of 0.2 nm/s to prepare a hole-transport layer.

In a nitrogen atmosphere, a glass sheet for sealing was bonded thereto with a UV curing resin to form a transverse current evaluation element. A voltage of 20 V was applied between the interdigitated electrodes of the element to measure the electric current as a transverse current. Table 1 shows the results.

### Examples 54 to 104 (Evaluation of Transverse Current of Compounds (D116) to (G702))

A transverse current evaluation element was prepared in the same manner as in Example 53 except that the compounds (D116) to (G702) purified by sublimation in Examples 2 to 52 were used instead of the compound (D68). Table 1 shows the transverse currents of the transverse current evaluation elements measured in the same manner as in Example 53.

### Comparative Examples 1 to 4 (Synthesis and Evaluation of Transverse Current of Compounds (a) to (d))

Known compounds (a) to (d) were synthesized and purified by sublimation.

Transverse current evaluation elements were prepared in the same manner as in Example 53 except that the compounds (a) to (d) were used instead of the compound (D4). Table 1 shows the transverse currents of the transverse current evaluation elements measured in the same manner as in Example 53.

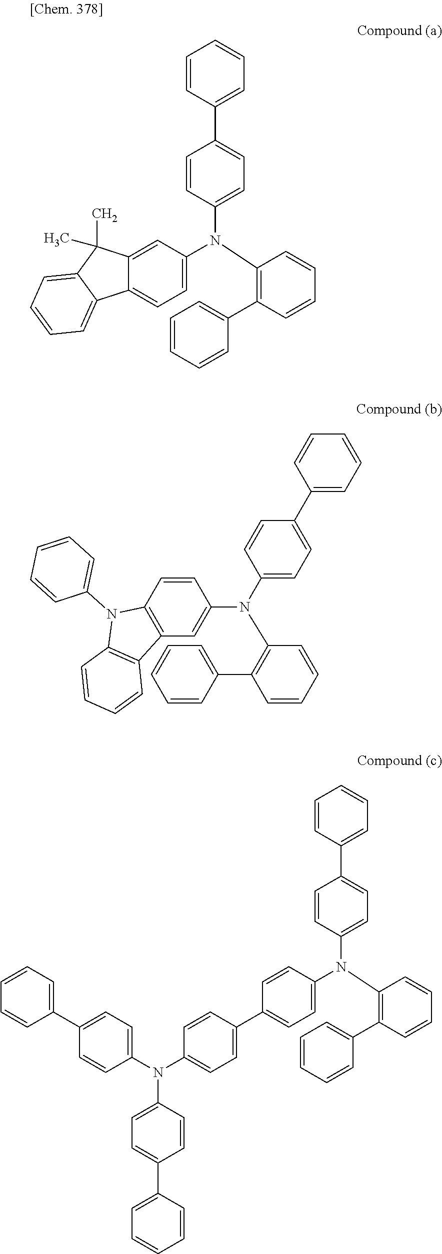 [SMILES: CC1(C)c2ccccc2-c2ccc(N(c3ccc(-c4ccccc4)cc3)c3ccccc3-c3ccccc3)cc21 ; c1ccc(-c2ccc(N(c3ccc4c(c3)c3ccccc3n4-c3ccccc3)c3ccccc3-c3ccccc3)cc2)cc1 ; c1ccc(-c2ccc(N(c3ccc(-c4ccccc4)cc3)c3ccc(-c4ccc(N(c5ccc(-c6ccccc6)cc5)c5ccccc5-c5ccccc5)cc4)cc3)cc2)cc1]

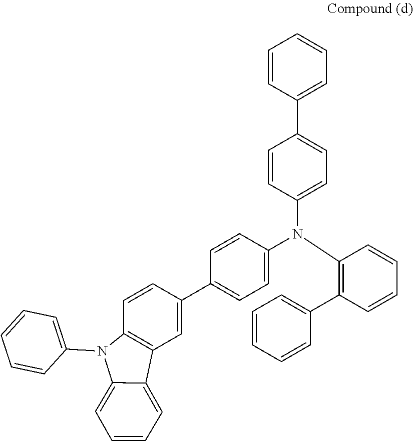 [SMILES: c1ccc(-c2ccc(N(c3ccc(-c4ccc5c(c4)c4ccccc4n5-c4ccccc4)cc3)c3ccccc3-c3ccccc3)cc2)cc1]

### Reference Examples 1 and 2 (Synthesis and Evaluation of Transverse Current of Compounds (e) and (f))

Known compounds (e) and (f) were synthesized and purified by sublimation.

Transverse current evaluation elements were prepared in the same manner as in Example 53 except that the compounds (e) and (f) were used instead of the compound (D4). Table 1 shows the transverse currents of the transverse current evaluation elements measured in the same manner as in Example 53.

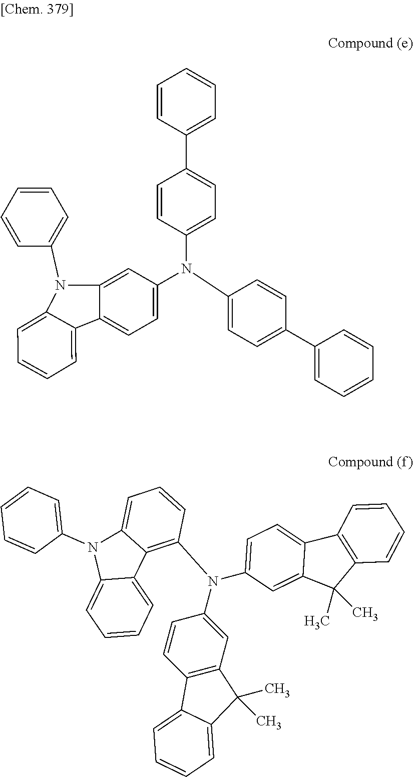 [SMILES: c1ccc(-c2ccc(N(c3ccc(-c4ccccc4)cc3)c3ccc4c5ccccc5n(-c5ccccc5)c4c3)cc2)cc1 ; CC1(C)c2ccccc2-c2ccc(N(c3ccc4c(c3)C(C)(C)c3ccccc3-4)c3cccc4c3c3ccccc3n4-c3ccccc3)cc21]

TABLE 1

| | | Transverse |
|---|---|---|
| | Compound | current (nA) |
| Example 53 | D68 | 99 |
| Example 54 | D116 | 39 |
| Example 55 | D166 | 164 |
| Example 56 | D169 | 138 |
| Example 57 | D180 | 74 |
| Example 58 | D184 | 96 |
| Example 59 | D188 | 122 |
| Example 60 | D194 | 101 |
| Example 61 | D273 | 88 |
| Example 62 | D278 | 32 |
| Example 63 | D285 | 76 |
| Example 64 | D288 | 64 |
| Example 65 | D289 | 95 |
| Example 66 | D291 | 63 |
| Example 67 | D312 | 26 |
| Example 68 | D336 | 24 |
| Example 69 | D350 | 16 |
| Example 70 | D352 | 28 |
| Example 71 | D357 | 3 |
| Example 72 | D360 | 19 |
| Example 73 | D429 | 111 |
| Example 74 | D657 | 180 |
| Example 75 | D757 | 105 |
| Example 76 | D805 | 80 |
| Example 77 | D818 | 1 |
| Example 78 | D844 | 81 |
| Example 79 | D850 | 46 |
| Example 80 | D853 | 87 |
| Example 81 | D859 | 11 |
| Example 82 | E103 | 106 |
| Example 83 | E107 | 131 |
| Example 84 | E111 | 83 |
| Example 85 | E161 | 169 |
| Example 86 | E169 | 177 |
| Example 87 | E179 | 142 |
| Example 88 | E228 | 166 |
| Example 89 | E264 | 8 |
| Example 90 | E268 | 80 |
| Example 91 | E303 | 12 |
| Example 92 | E329 | 3 |
| Example 93 | E330 | 30 |
| Example 94 | E341 | 25 |
| Example 95 | E674 | 5 |
| Example 96 | E770 | 169 |
| Example 97 | F166 | 26 |
| Example 98 | F228 | 30 |
| Example 99 | F320 | 4 |
| Example 100 | F424 | 29 |
| Example 101 | F839 | 6 |
| Example 102 | F901 | 0.9 |
| Example 103 | G360 | 0.8 |
| Example 104 | G702 | 2 |
| Comparative | Compound (a) | 353 |
| example 1 | | |
| Comparative | Compound (b) | 416 |
| example 2 | | |
| Comparative | Compound (c) | 354 |
| example 3 | | |
| Comparative | Compound (d) | 314 |
| example 4 | | |
| Reference | Compound (e) | 502 |
| example 1 | | |
| Reference | Compound (f) | 320 |
| example 2 | | |

### Example 105 (Evaluation of Transverse Current of Mixed Film of Compound (D180) and Compound (d))

A transverse current evaluation element was prepared in the same manner as in Example 53 except that a mixed film of the compound (D180) and the compound (d) (weight ratio: 50:50) was used instead of the compound (D4). Table 2 shows the transverse current measured in the same manner as in Example 53.

### Example 106 (Evaluation of Transverse Current of Mixed Film of Compound (D853) and Compound (d))

A transverse current evaluation element was prepared in the same manner as in Example 53 except that a mixed film of the compound (D853) and the compound (d) (weight ratio: 50:50) was used instead of the compound (D4). Table 2 shows the transverse current measured in the same manner as in Example 53.

### Example 107 (Evaluation of Transverse Current of Mixed Film of Compound (E111) and Compound (c))

A transverse current evaluation element was prepared in the same manner as in Example 53 except that a mixed film of the compound (E111) and the compound (c) (weight ratio: 50:50) was used instead of the compound (D4). Table 2 shows the transverse current measured in the same manner as in Example 53.

### Example 108 (Evaluation of Transverse Current of Mixed Film of Compound (E228) and Compound (c))

A transverse current evaluation element was prepared in the same manner as in Example 53 except that a mixed film of the compound (E111) and the compound (c) (weight ratio: 50:50) was used instead of the compound (D4). Table 2 shows the transverse current measured in the same manner as in Example 53.

### Example 109 (Evaluation of Transverse Current of Mixed Film of Compound (F320) and Compound (e))

A transverse current evaluation element was prepared in the same manner as in Example 53 except that a mixed film of the compound (F320) and the compound (e) (weight ratio: 30:70) was used instead of the compound (D4). Table 2 shows the transverse current measured in the same manner as in Example 53.

TABLE 2

| | | Transverse |
|---|---|---|
| | Compound | current (nA) |
| Example 105 | (D180)/(d) | 190 |
| Example 106 | (D853)/(d) | 180 |
| Example 107 | (E111)/(c) | 220 |
| Example 108 | (E228)/(c) | 200 |
| Example 109 | (F320)/(e) | 274 |

### Examples of Organic Electroluminescent Elements

The following are the preparation of organic electroluminescent elements and the structural formulae and abbreviations for compounds used.

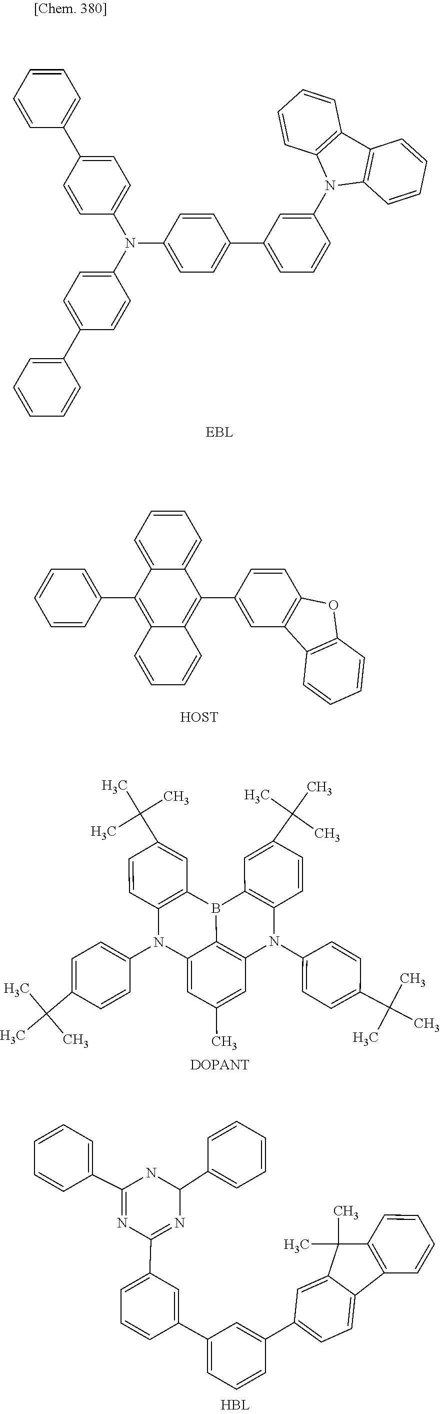

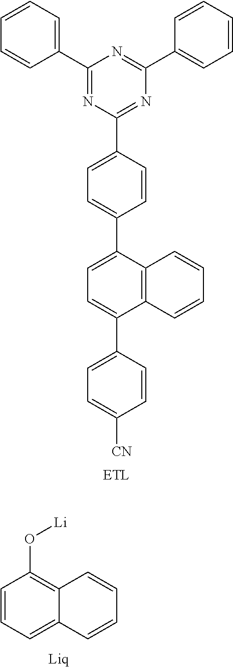

### Example 110 (Evaluation of Element of Compound (D273))

A glass substrate with an ITO transparent electrode was prepared in which an indium tin oxide (ITO) film (film thickness: 110 nm) with a width of 2 mm was patterned in a stripe pattern. The substrate was then washed with isopropyl alcohol and was then surface-treated by ozone ultraviolet cleaning. The glass substrate was introduced into a vacuum evaporation bath, and the pressure was reduced to 1.0×10⁻⁴ Pa. Each layer was then formed in the following order under their respective film-forming conditions.

### (Preparation of Hole-Injection Layer)

The compound (D273) and 1,2,3-tris[(4-cyano-2,3,5,6-tetrafluorophenyl)methylene]cyclopropane were formed into a film with a thickness of 10 nm at a ratio of 99:1 (mass ratio) to prepare a hole-injection layer.

### (Preparation of Hole-Transport Layer)

The compound (D273) was formed into a film with a thickness of 100 nm at a rate of 0.2 nm/s to prepare a hole-transport layer.

### (Preparation of Electron-Blocking Layer)

EBL was formed into a film with a thickness of 5 nm at a rate of 0.15 nm/s to prepare an electron-blocking layer.

### (Preparation of Light-Emitting Layer)

HOST and DOPANT were formed into a film with a thickness of 20 nm at a ratio of 95:5 (mass ratio) to prepare a light-emitting layer. The film-forming rate was 0.18 nm/s.

### (Preparation of Electron-Transport Layer)

HBL was formed into a film with a thickness of 6 nm at a rate of 0.05 nm/s to prepare a first electron-transport layer.

### (Preparation of Electron-Injection Layer)

ETL and Liq were formed into a film with a thickness of 25 nm at a ratio of 50:50 (mass ratio) to prepare a second electron-transport layer. The film-forming rate was 0.15 nm/s.

### (Preparation of Negative Electrode)

Finally, a metal mask was disposed perpendicularly to the ITO stripes on the substrate to form a negative electrode. The negative electrode was formed in a three-layer structure by depositing ytterbium, silver/magnesium (mass ratio: 9/1) and silver in this order at 2 nm, 12 nm and 90 nm, respectively. The deposition rate of ytterbium was 0.02 nm/s, the deposition rate of silver/magnesium was 0.5 nm/s, and the deposition rate of silver was 0.2 nm/s.

In this manner, an organic electroluminescent element with a luminous area of 4 mm² was prepared.

Furthermore, the element was sealed in a nitrogen atmosphere glove box with an oxygen and water concentration of 1 ppm or less. The sealing was performed using a glass sealing cap, a film-formed substrate (element) and a UV-curable epoxy resin (manufactured by Moresco Corporation).

An electric current of 10 mA/cm² was applied to the element thus prepared to measure the voltage and luminous efficiency. Table 3 shows the results.

### Examples 111 to 116 (Evaluation of Elements of Compound (D336), Compound (D350), Compound (E103), Compound (E264), Compound (F228) and Compound (F901))

Organic electroluminescent elements were prepared in the same manner as in Example 110 except that the compound (D336), the compound (D350), the compound (E103), the compound (E264), the compound (F228) and the compound (F901) were used instead of the compound (D273). Table 3 shows the results.

### Comparative Examples 5 to 7 (Evaluation of Element of Compound (g), Compound (h) and Compound (i))

Organic electroluminescent elements were prepared in the same manner as in Example 110 except that the compound (g), the compound (h) and the compound (1) were used instead of the compound (D273). Table 3 shows the results.

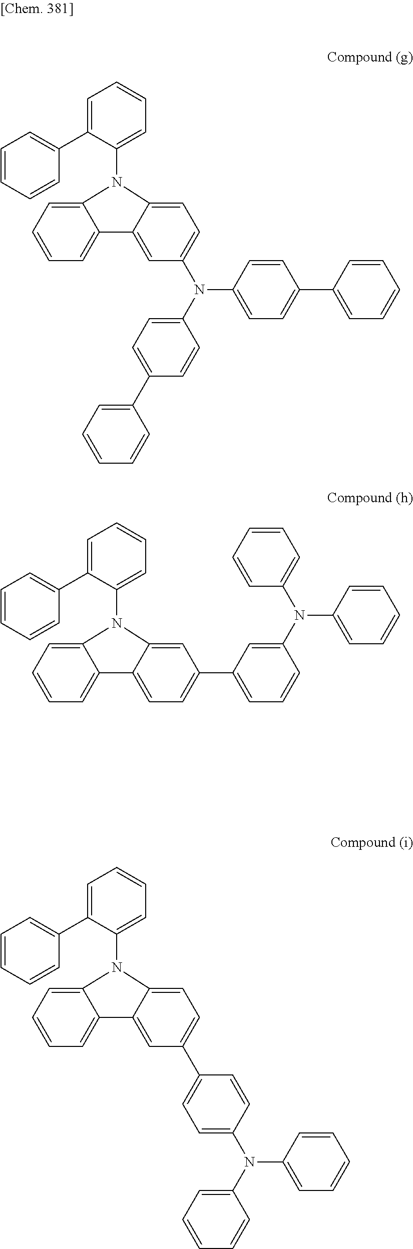 [SMILES: c1ccc(-c2ccc(N(c3ccc(-c4ccccc4)cc3)c3ccc4c(c3)c3ccccc3n4-c3ccccc3-c3ccccc3)cc2)cc1 ; c1ccc(-c2ccccc2-n2c3ccccc3c3ccc(-c4cccc(N(c5ccccc5)c5ccccc5)c4)cc32)cc1 ; c1ccc(-c2ccccc2-n2c3ccccc3c3cc(-c4ccc(N(c5ccccc5)c5ccccc5)cc4)ccc32)cc1]

TABLE 3

| | | | Luminous |
|---|---|---|---|
| | | Voltage | efficiency |
| | Compound | (V) | (cd/A) |
| Example 110 | (D273) | 3.4 | 5.2 |
| Example 111 | (D336) | 3.5 | 5.4 |
| Example 112 | (D350) | 3.6 | 5.3 |
| Example 113 | (E103) | 3.4 | 5.0 |
| Example 114 | (E264) | 3.6 | 5.0 |
| Example 115 | (F228) | 3.6 | 5.3 |
| Example 116 | (F901) | 3.5 | 5.2 |
| Comparative | Compound (g) | 3.7 | 4.2 |
| example 5 | | | |
| Comparative | Compound (h) | 4.0 | 4.5 |
| example 6 | | | |
| Comparative | Compound (i) | 3.9 | 4.7 |
| example 7 | | | |

While the present invention has been described in detail with reference to specific embodiments, it is apparent to a person skilled in the art that various alterations and modifications may be made to the embodiments without departing from the essence and scope of the present invention.

The entire contents of the description, claims, drawings and abstract of Japanese Patent Application No. 2021-61639 filed on Mar. 31, 2021 are incorporated herein by reference as the disclosure of the description of the present invention.

### REFERENCE SYMBOLS

1 substrate
2 positive electrode
3 hole-injection layer
4 hole-transport layer
5 electron-blocking layer
6 light-emitting layer
7 electron-transport layer
8 electron-injection layer
9 negative electrode

1. A transverse current suppressing material for an organic electroluminescent element, represented by the formula (1):
A-B [Chem. 1]
wherein
A is represented by the formula (2) or (3); and
B is represented by the formula (4); 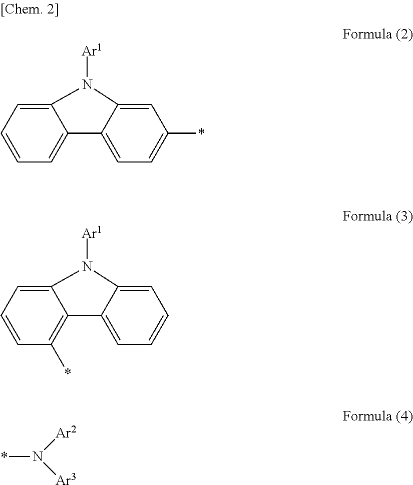
wherein
Ar¹ to Ar³ each independently denote
an optionally substituted monocyclic, linked or fused aromatic hydrocarbon group with 6 to 30 carbon atoms or
an optionally substituted monocyclic, linked or fused heteroaromatic group with 3 to 30 carbon atoms;
at least one of Ar¹ to Ar³ denotes a group represented by any one of the formulae (5) to (21); 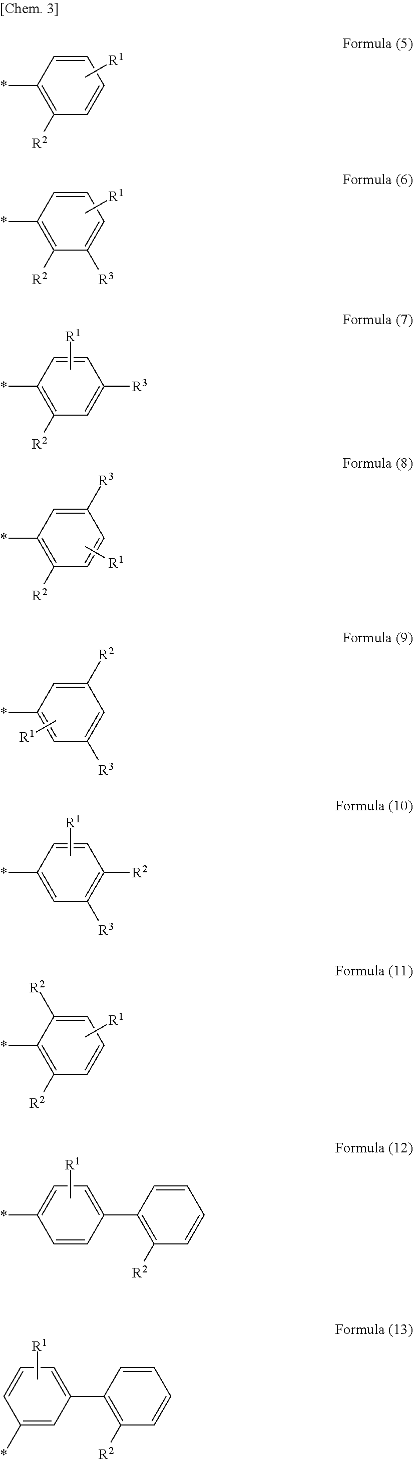 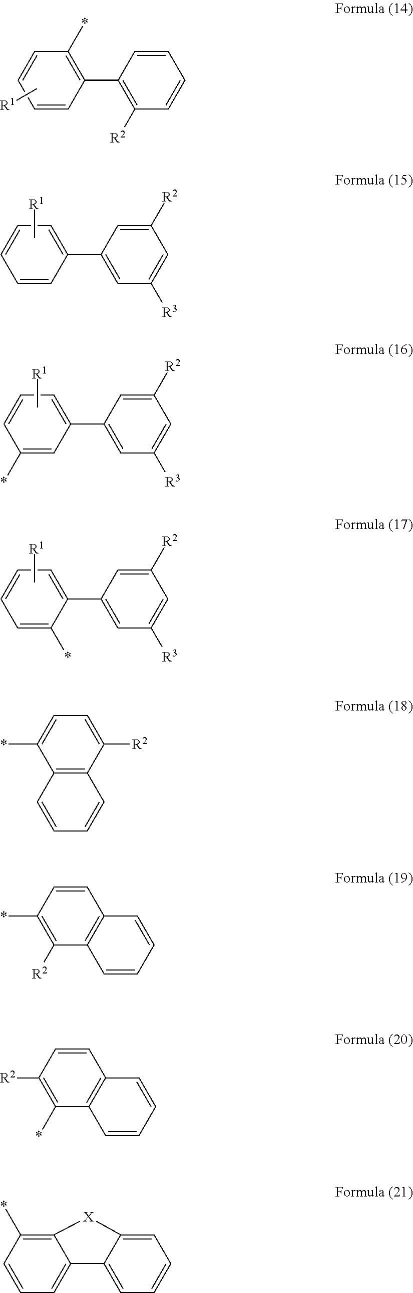
wherein
R¹ denotes a methyl group or a hydrogen atom;
R² and R³ each independently denote a phenyl group, a biphenylyl group, a naphthyl group, a phenanthryl group, a dibenzofuranyl group or a dibenzothienyl group, and is optionally substituted with a methyl group, and
X denotes an oxygen atom or a sulfur atom.

2. The transverse current suppressing material for the organic electroluminescent element according to claim 1, wherein Ar¹ denotes a group represented by any one of the formulae (5) to (21).

3. The transverse current suppressing material for the organic electroluminescent element according to claim 1, wherein both Ar¹ and Ar² denote a group represented by any one of the formulae (5) to (21).

4. The transverse current suppressing material for the organic electroluminescent element according to claim 1, wherein
Ar¹ to Ar³ each independently denote
(i) a phenyl group, a biphenylyl group, a terphenylyl group, a naphthyl group, a fluorenyl group, a spirobifluorenyl group, a benzofluorenyl group, a phenanthryl group, a fluoranthenyl group, a triphenylenyl group, an anthryl group, a pyrenyl group, a dibenzofuranyl group or a dibenzothienyl group,
(ii) the group represented by (i) substituted with one or more groups selected from the group consisting of a methyl group, an ethyl group, a methoxy group, an ethoxy group, a cyano group, a deuterium atom, a fluorine atom, a phenyl group, a biphenylyl group, a naphthyl group, a phenanthryl group, a triphenylsilyl group, a carbazolyl group, a dibenzothienyl group and a dibenzofuranyl group or
(iii) a group represented by any one of the formulae (5) to (21).

5. The transverse current suppressing material for the organic electroluminescent element according to claim 1, wherein at least one of Ar¹ to Ar³ denotes a group represented by any one of the formulae (Y1) to (Y298).

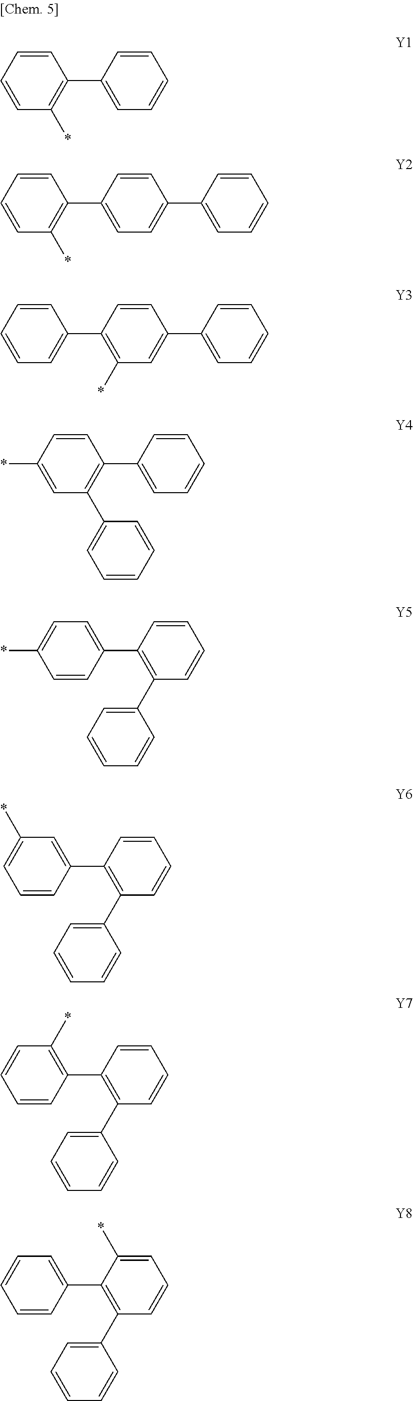

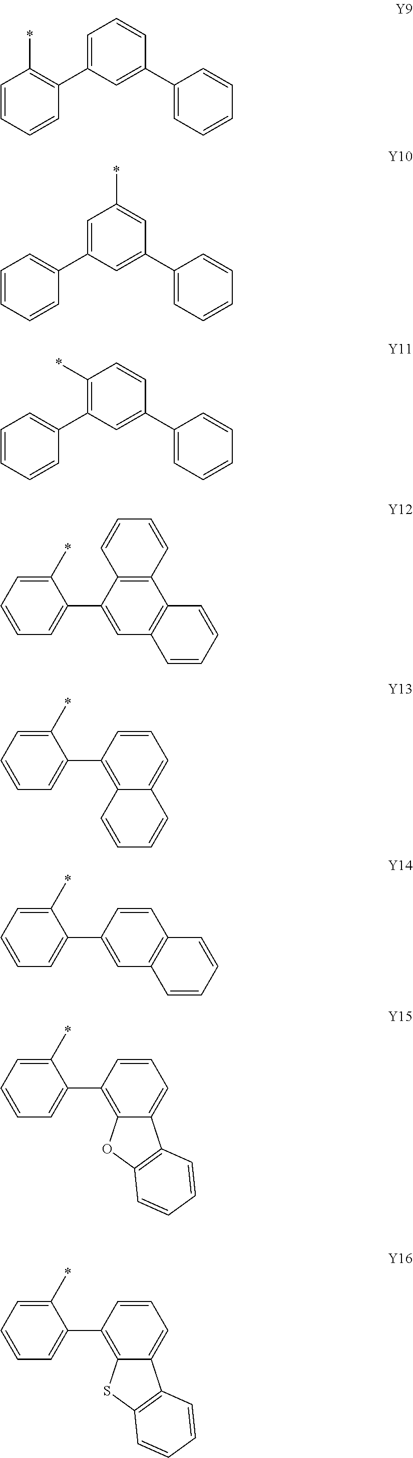

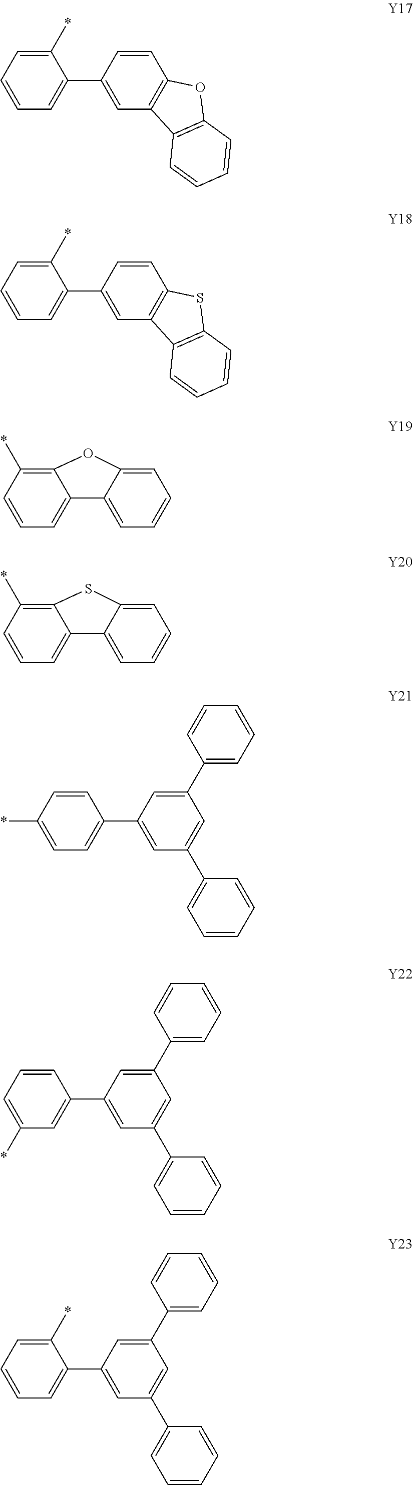

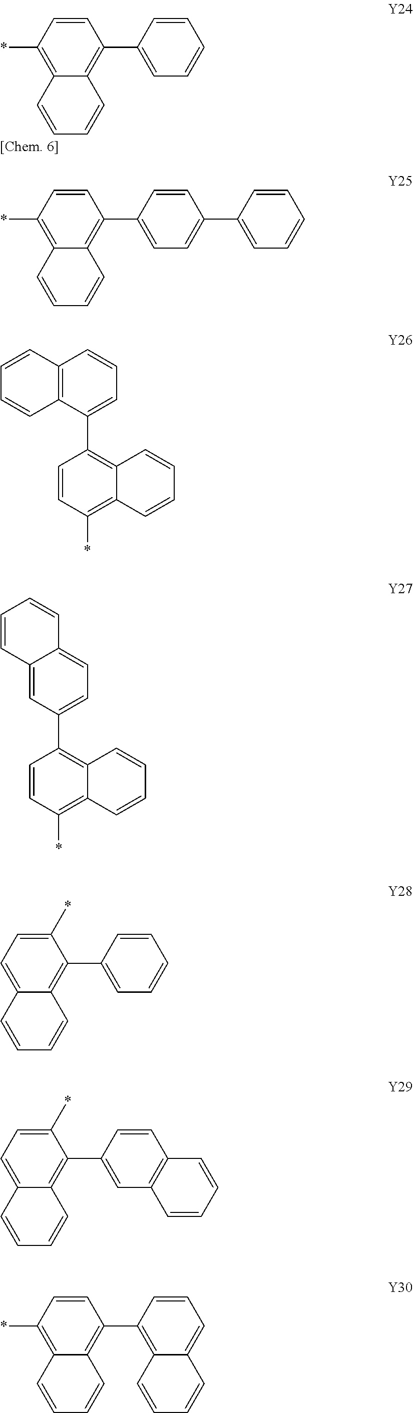

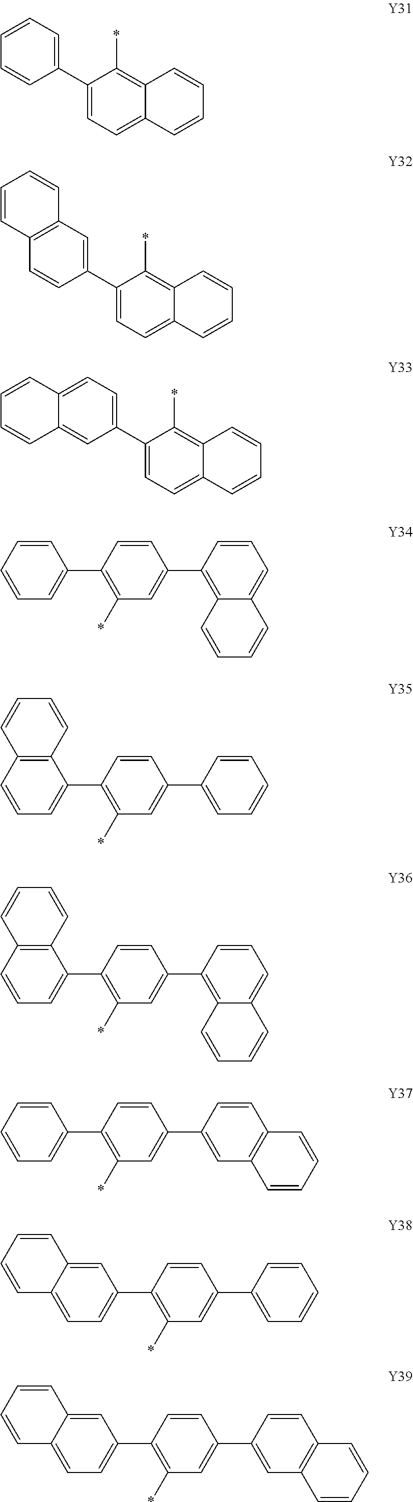

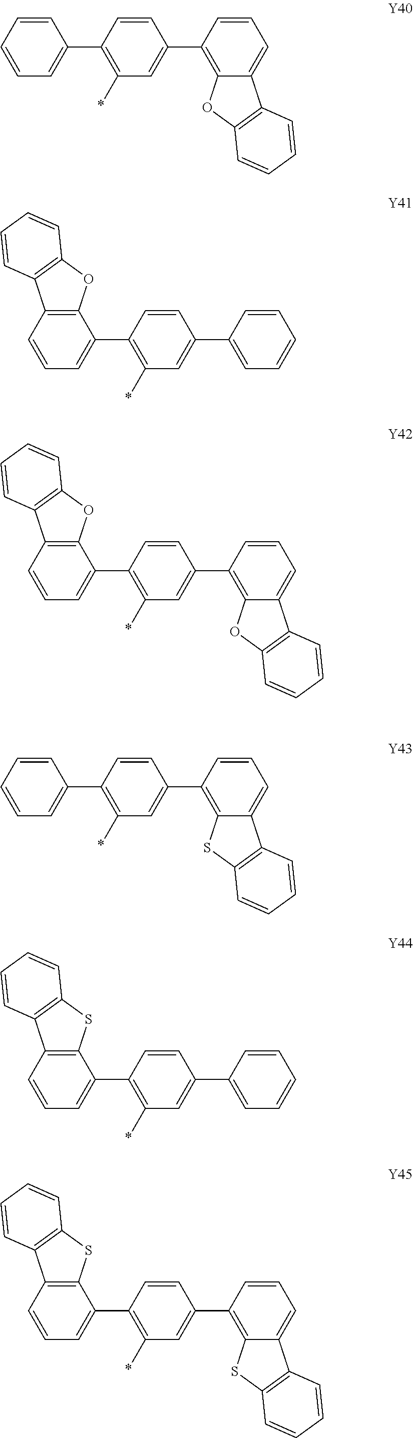

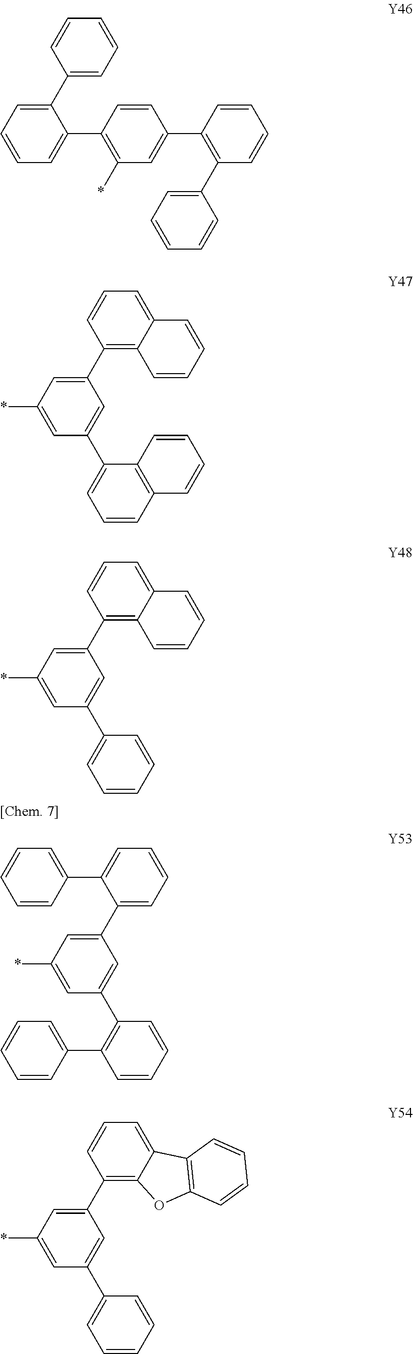

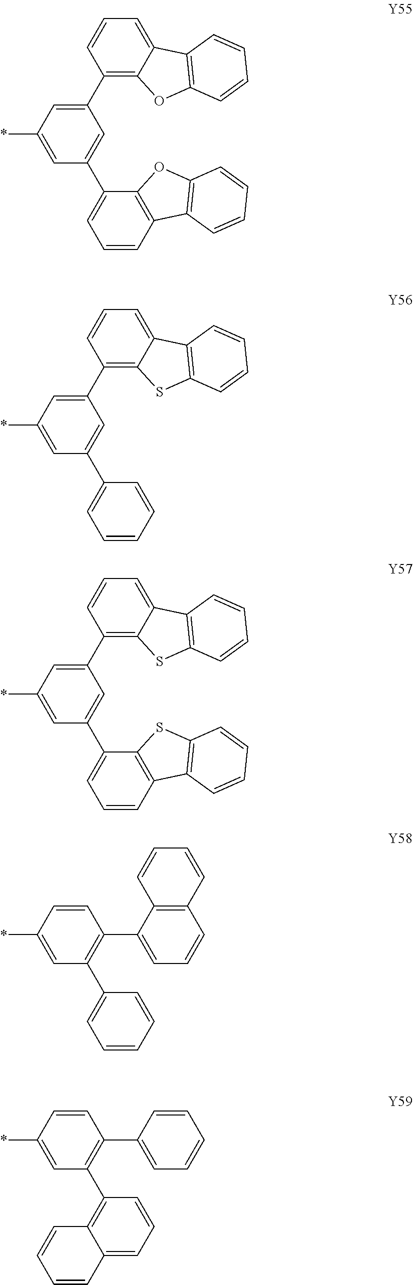

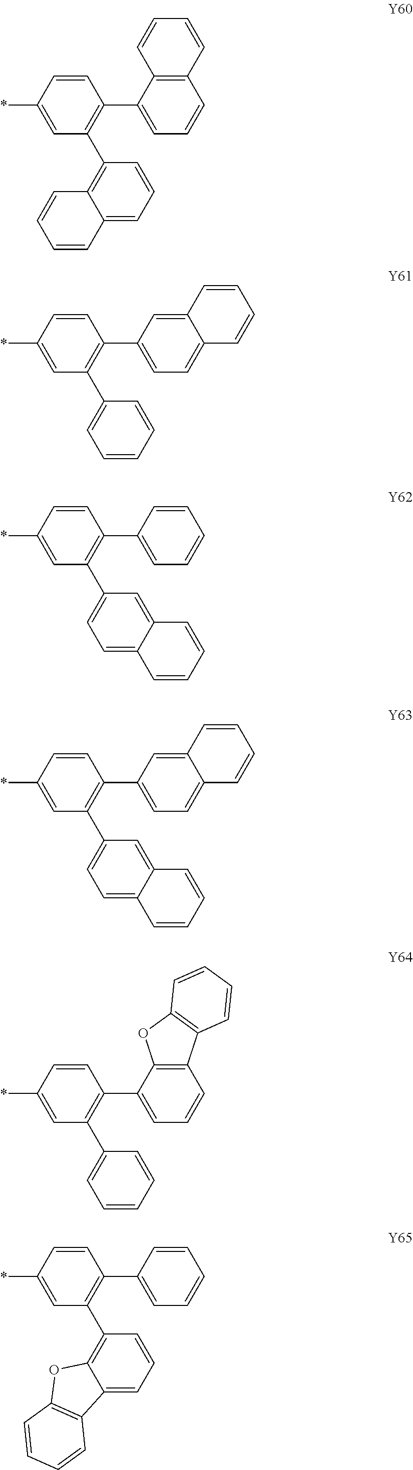

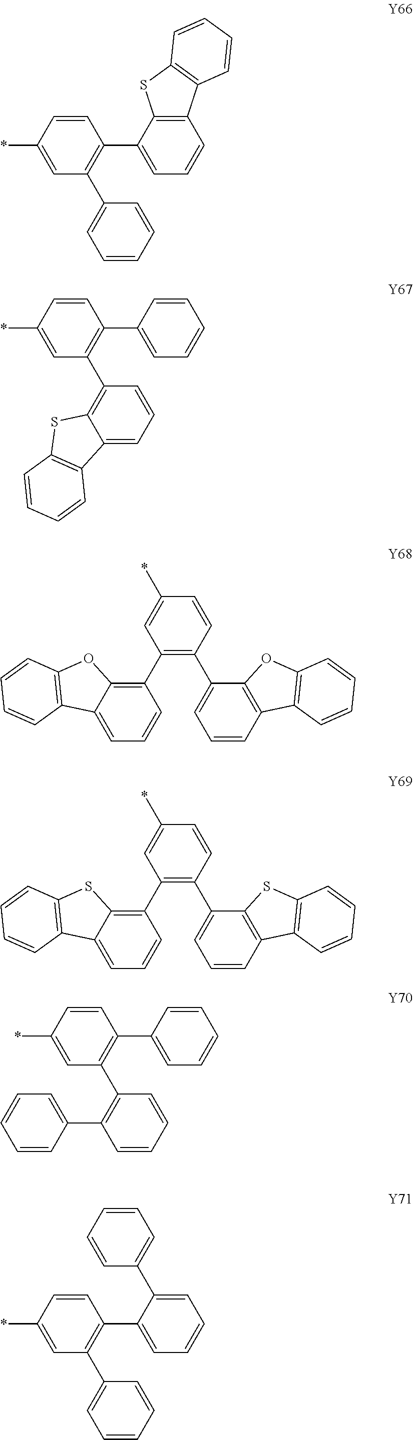

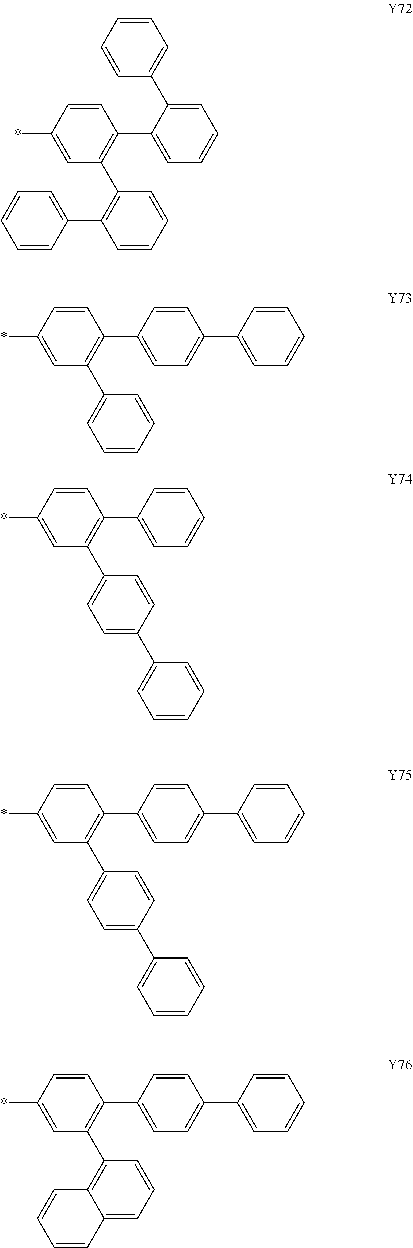

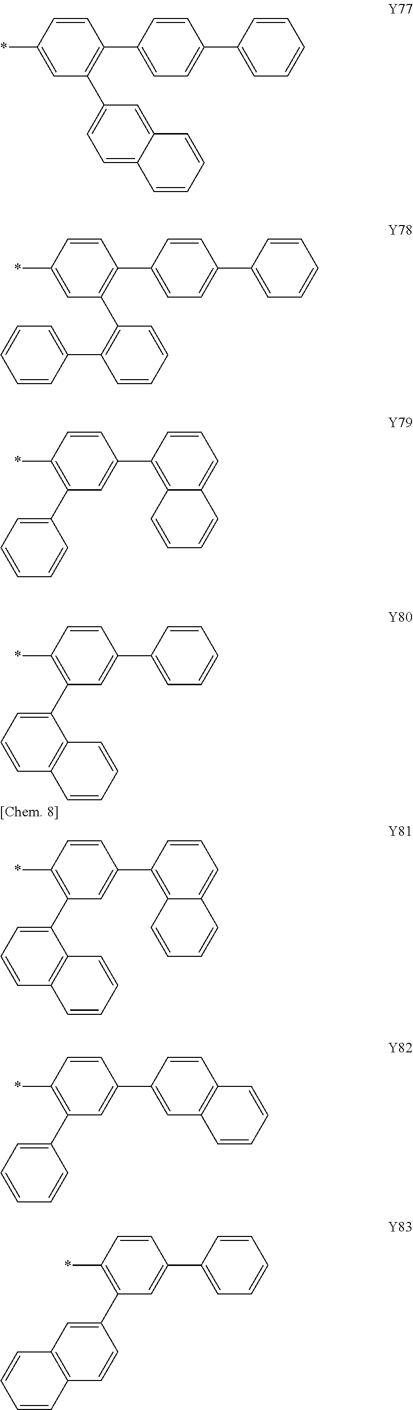

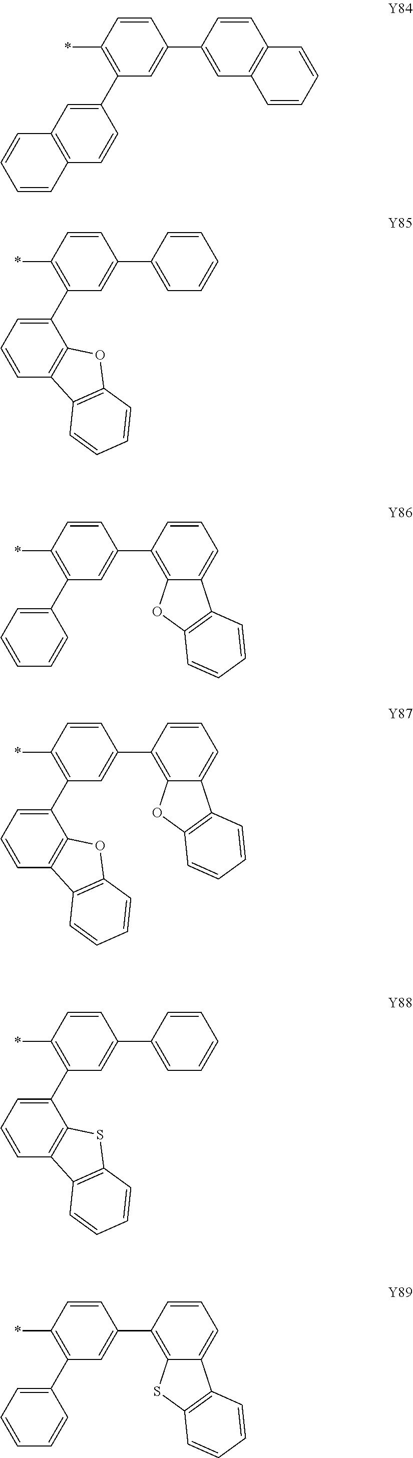

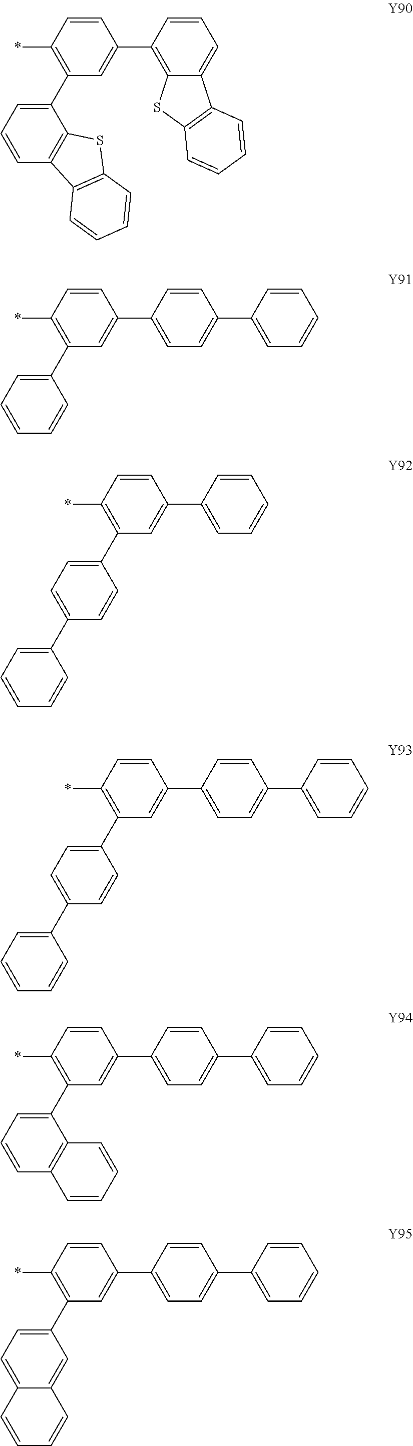

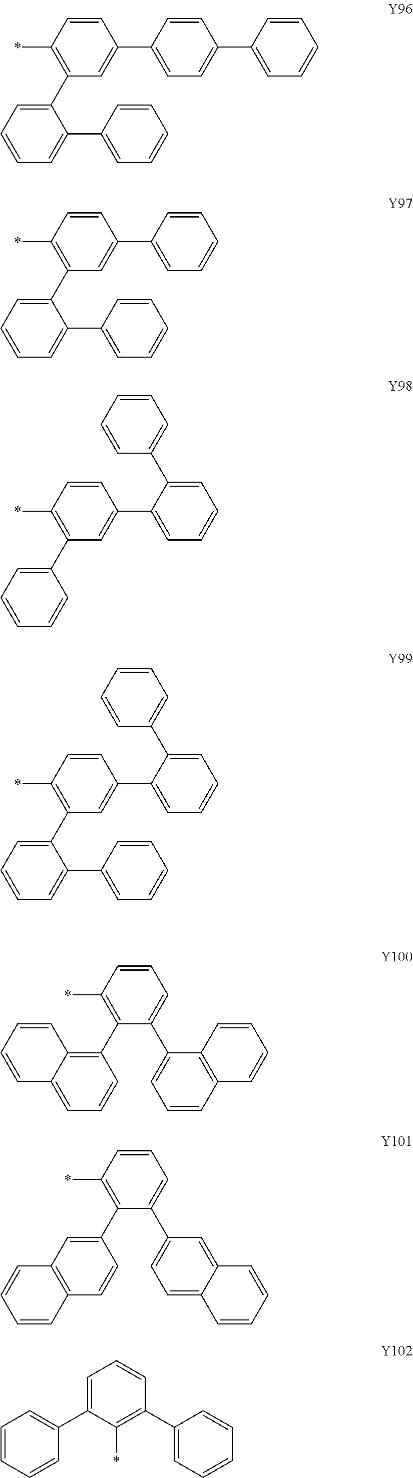

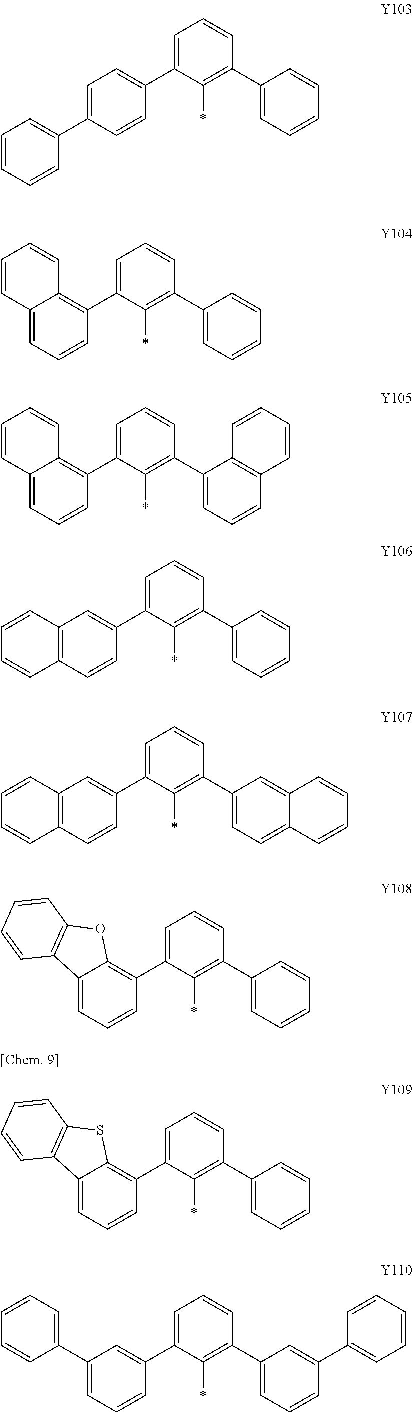

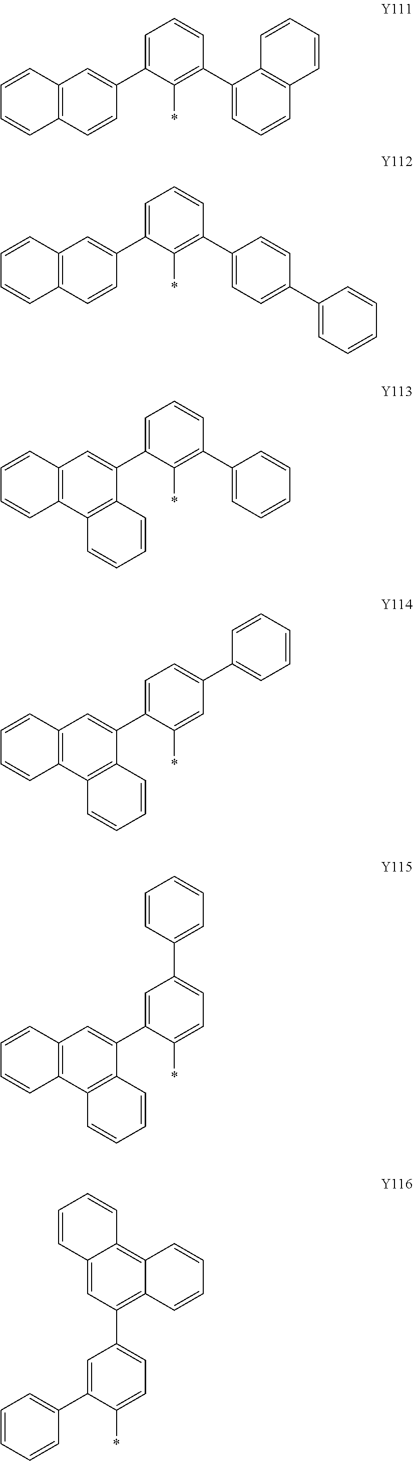 [SMILES: *c1c(-c2ccc3ccccc3c2)cccc1-c1cccc2ccccc12 ; *c1c(-c2ccc(-c3ccccc3)cc2)cccc1-c1ccc2ccccc2c1 ; *c1c(-c2ccccc2)cccc1-c1cc2ccccc2c2ccccc12 ; *c1cc(-c2ccccc2)ccc1-c1cc2ccccc2c2ccccc12 ; *c1ccc(-c2ccccc2)cc1-c1cc2ccccc2c2ccccc12 ; *c1ccc(-c2cc3ccccc3c3ccccc23)cc1-c1ccccc1]

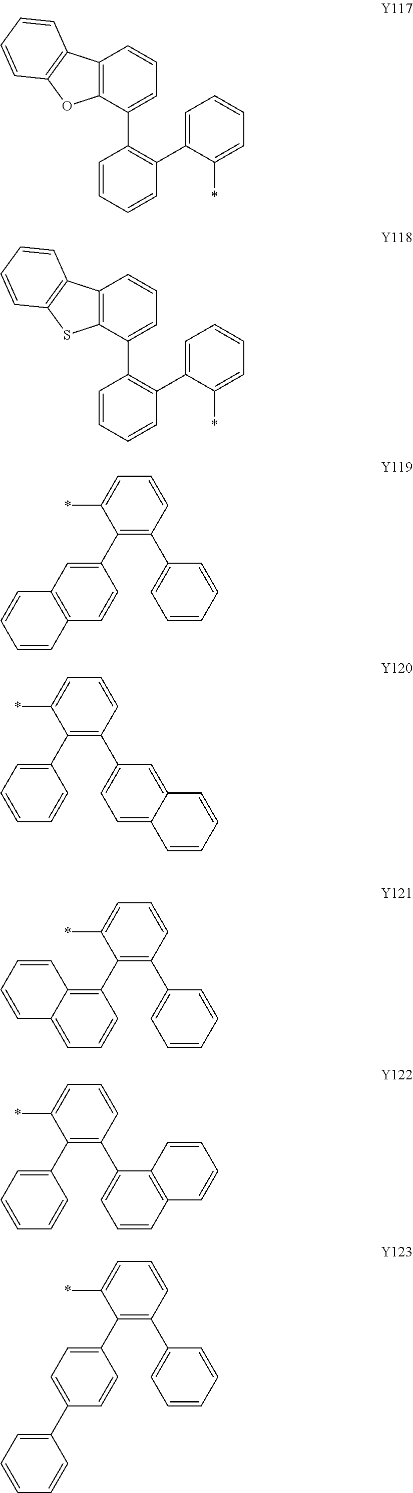

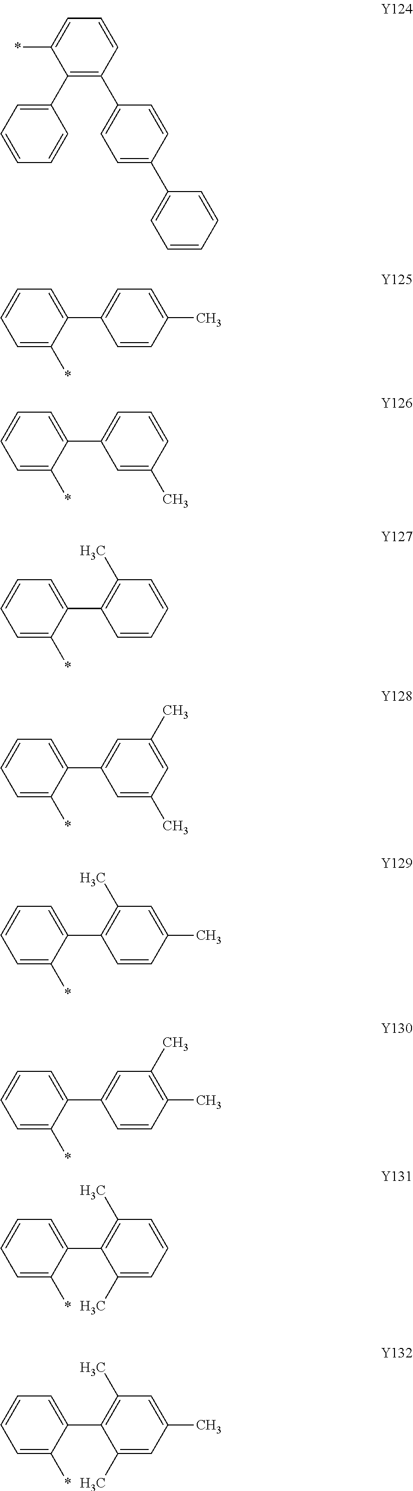

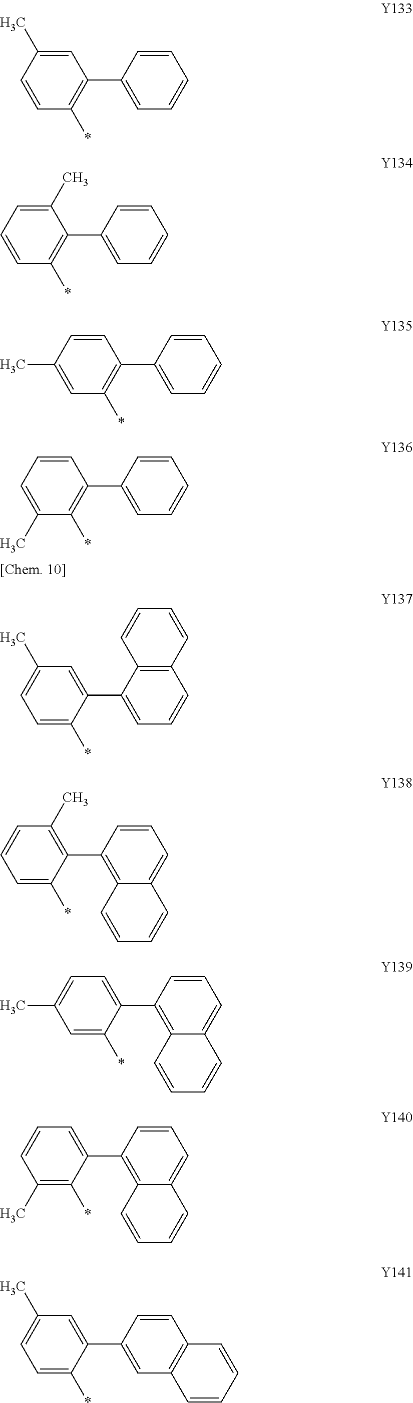

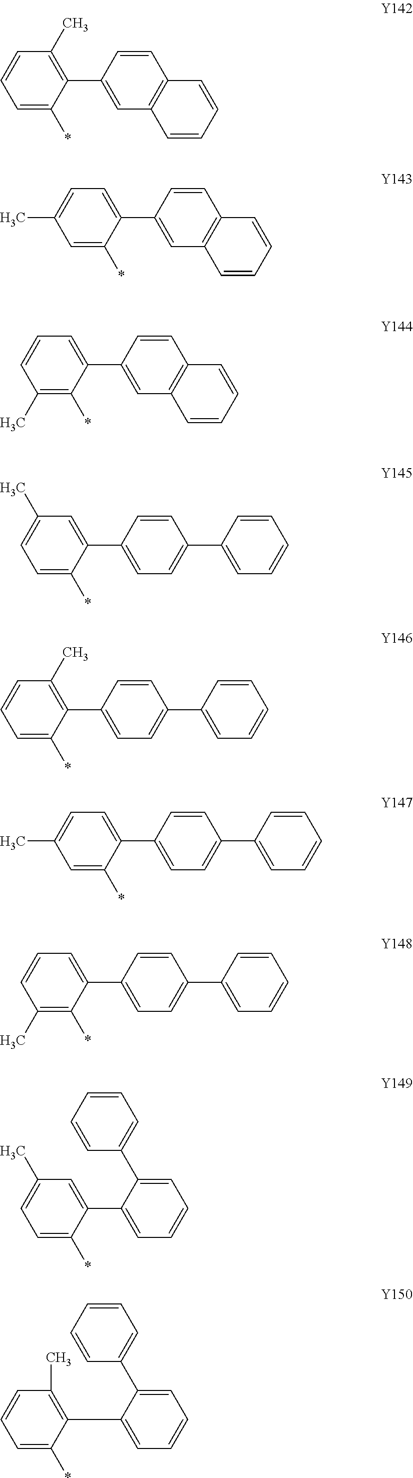

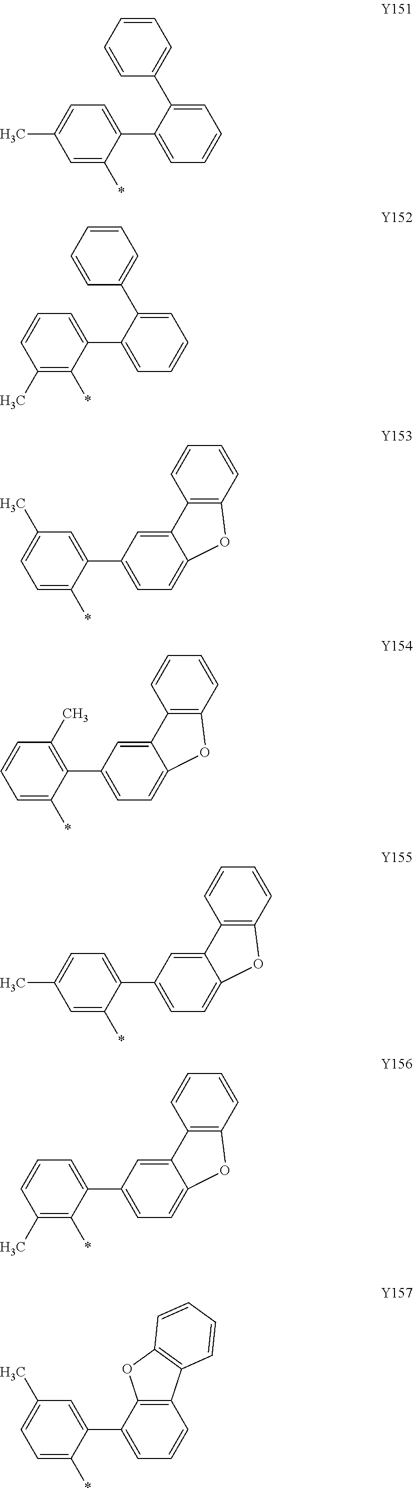

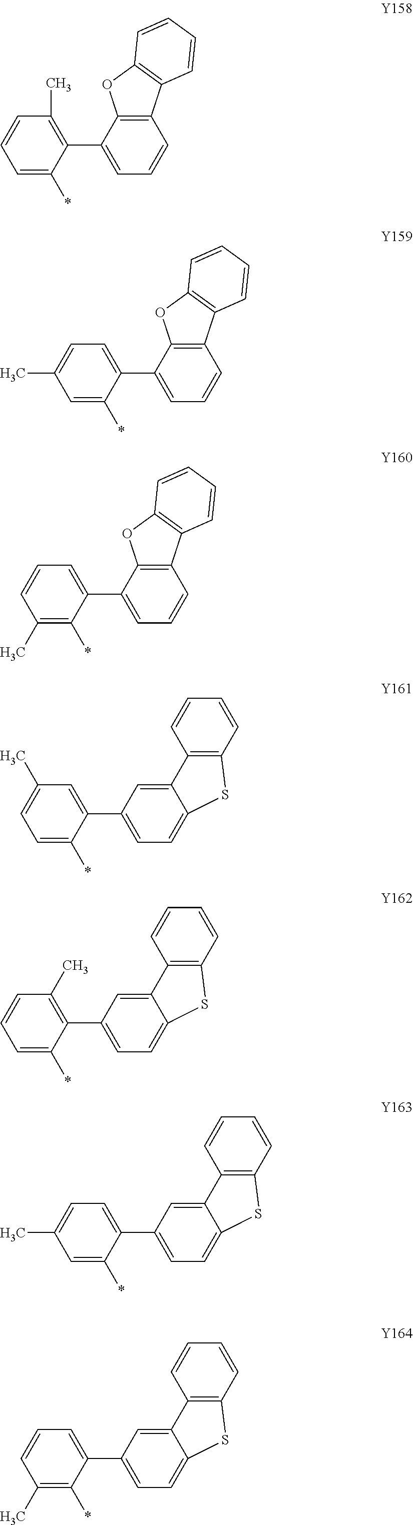

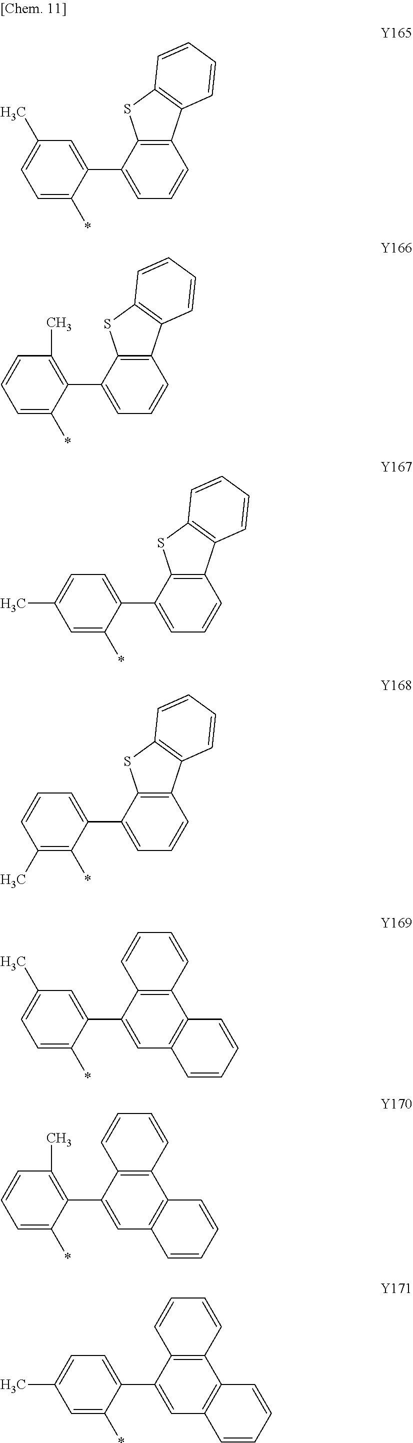

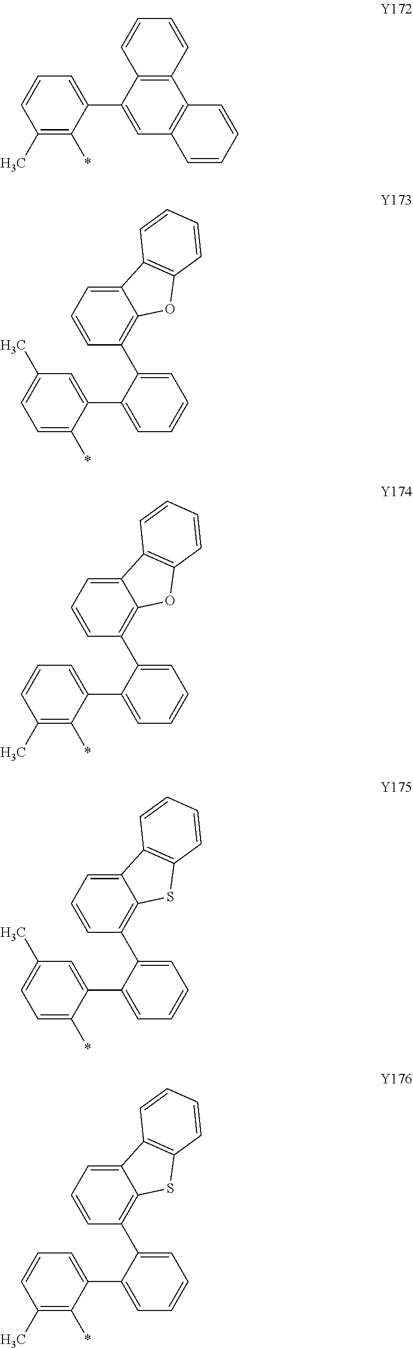 [SMILES: *c1c(C)cccc1-c1cc2ccccc2c2ccccc12 ; *c1ccc(C)cc1-c1ccccc1-c1cccc2c1oc1ccccc12 ; *c1c(C)cccc1-c1ccccc1-c1cccc2c1oc1ccccc12 ; *c1ccc(C)cc1-c1ccccc1-c1cccc2c1sc1ccccc12 ; *c1c(C)cccc1-c1ccccc1-c1cccc2c1sc1ccccc12]

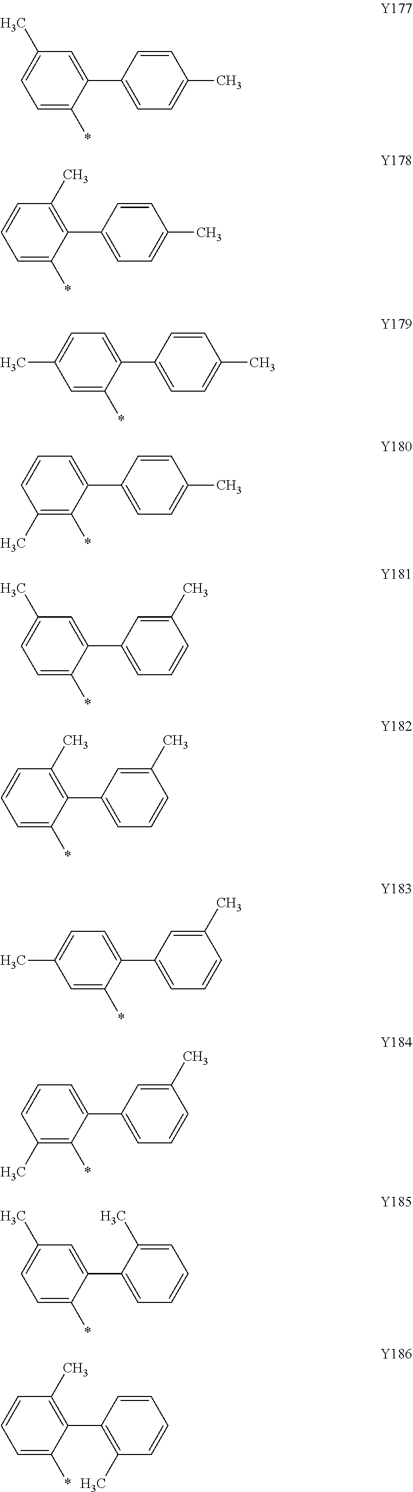

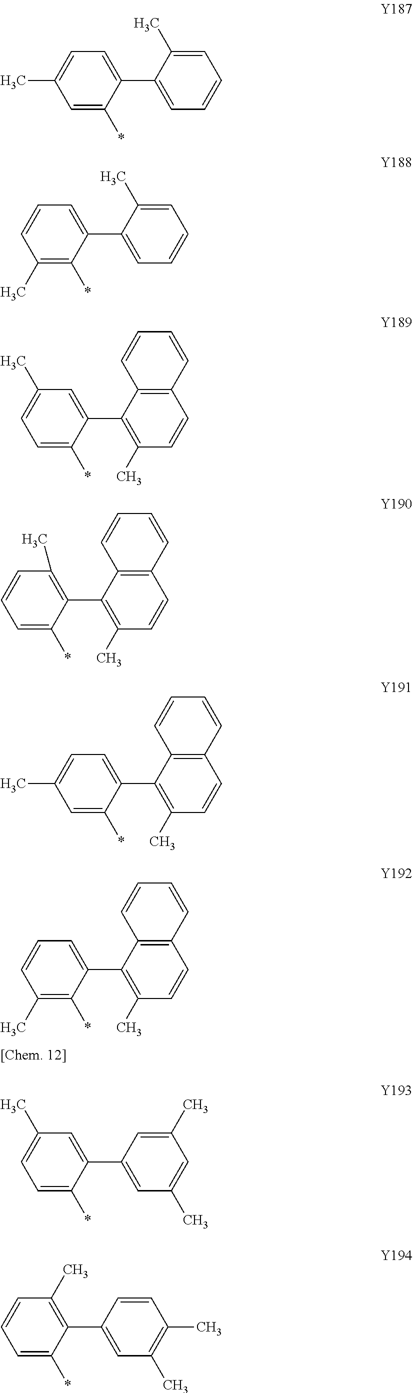

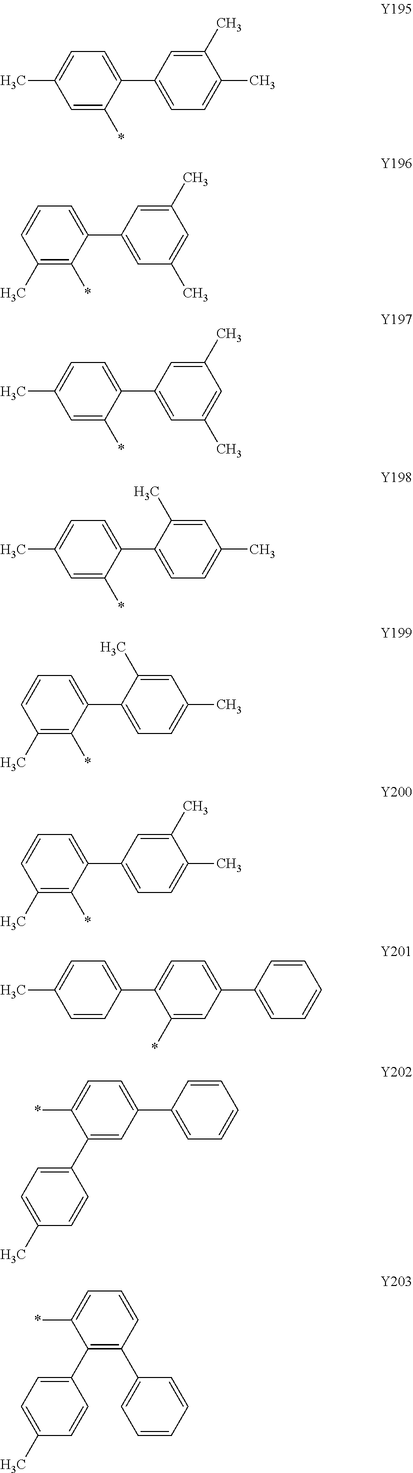

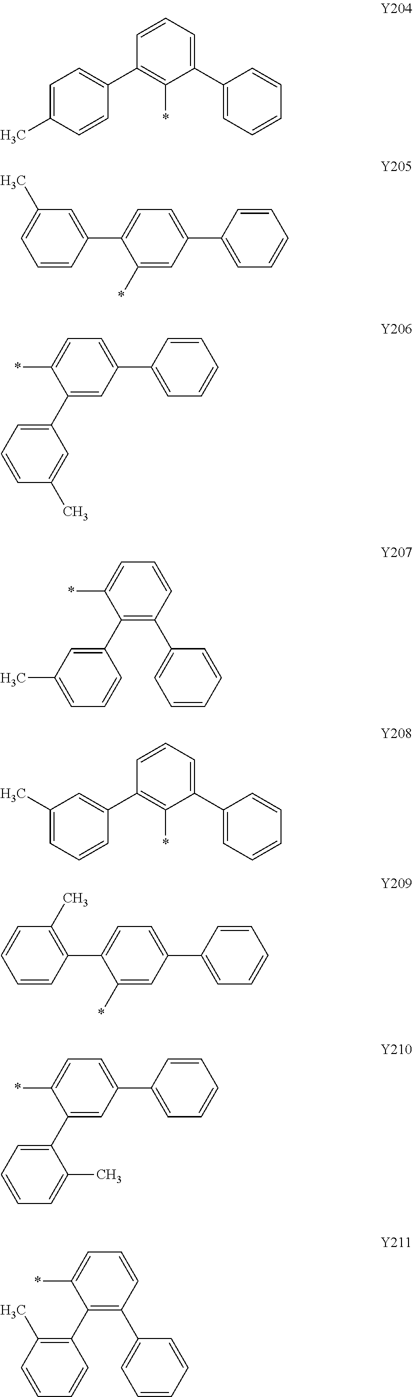

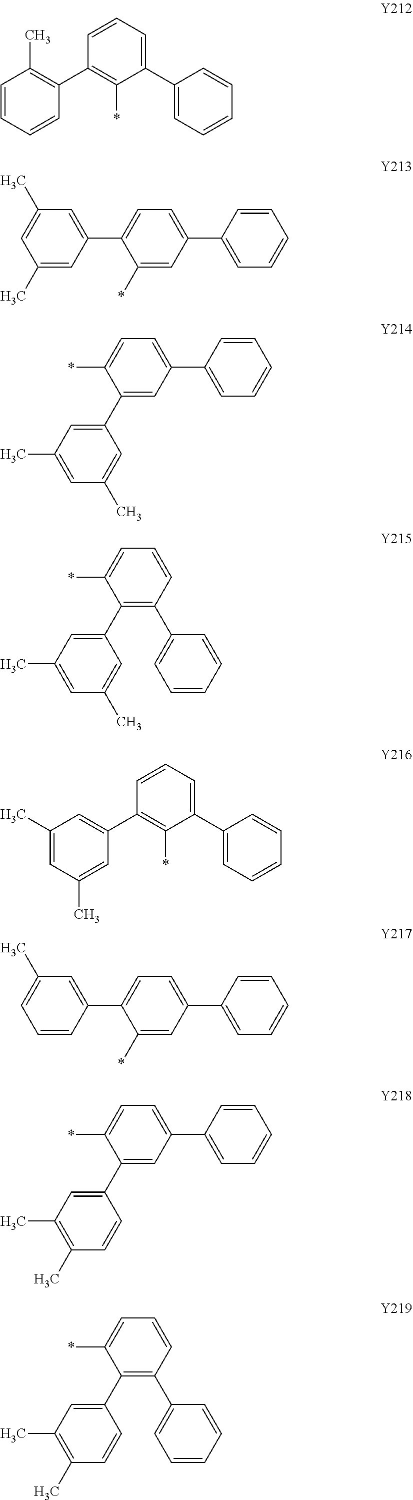

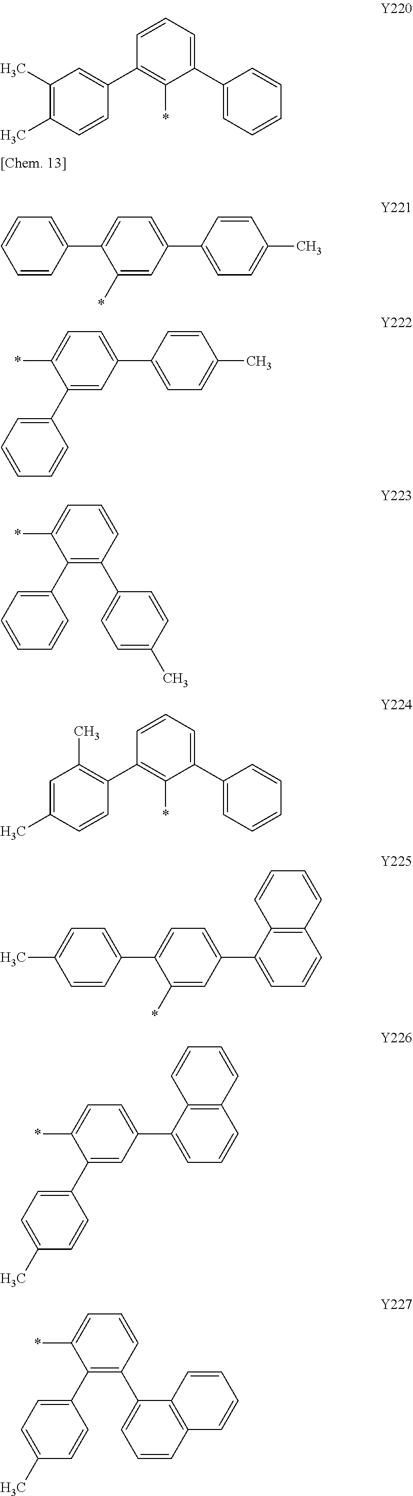

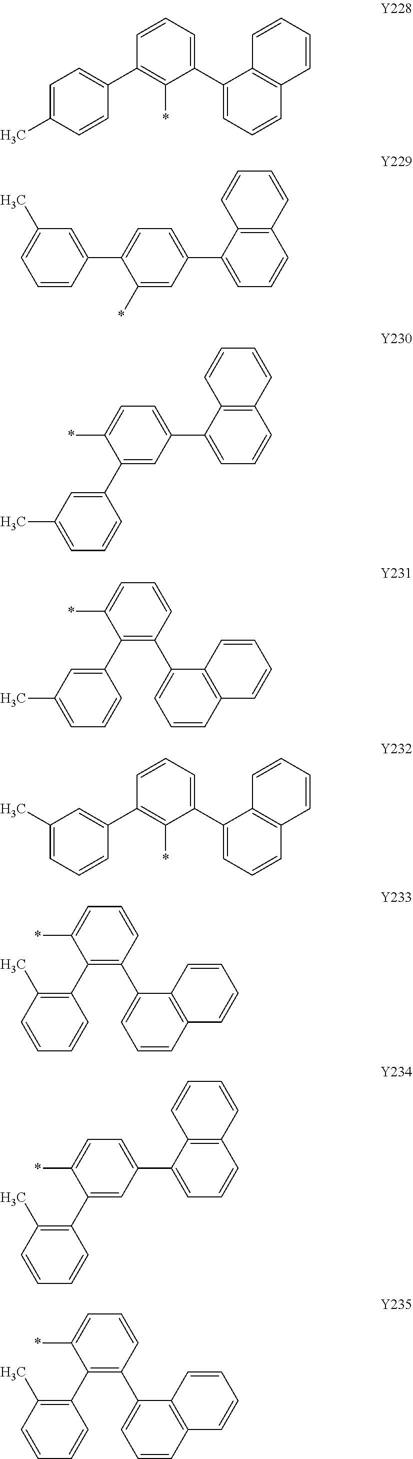

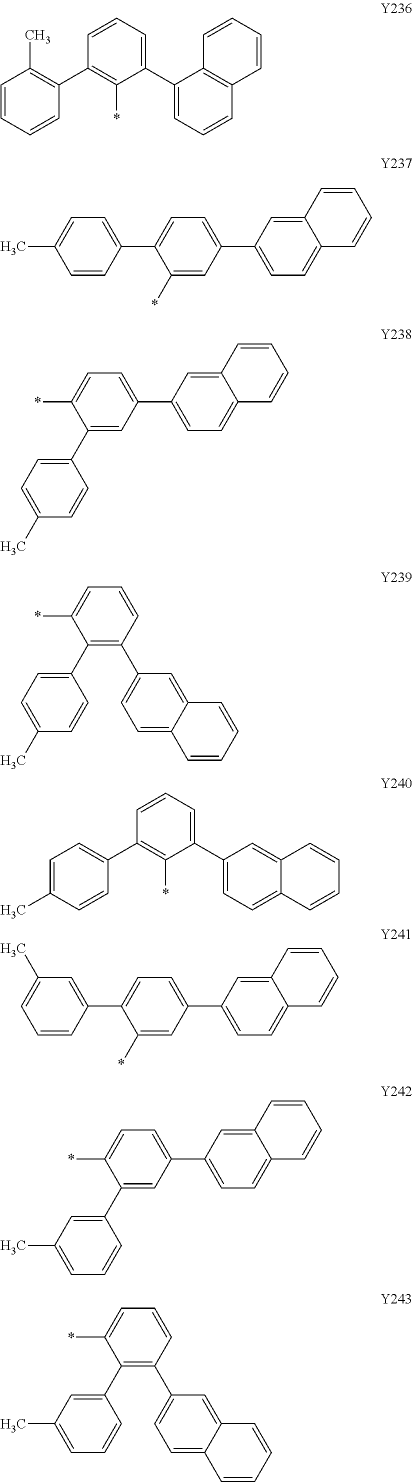

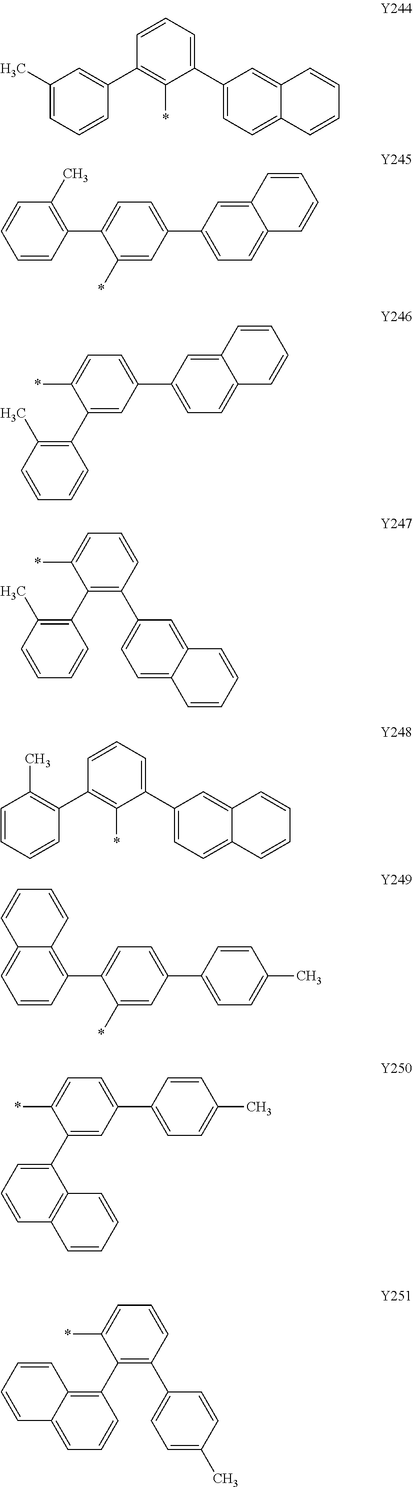

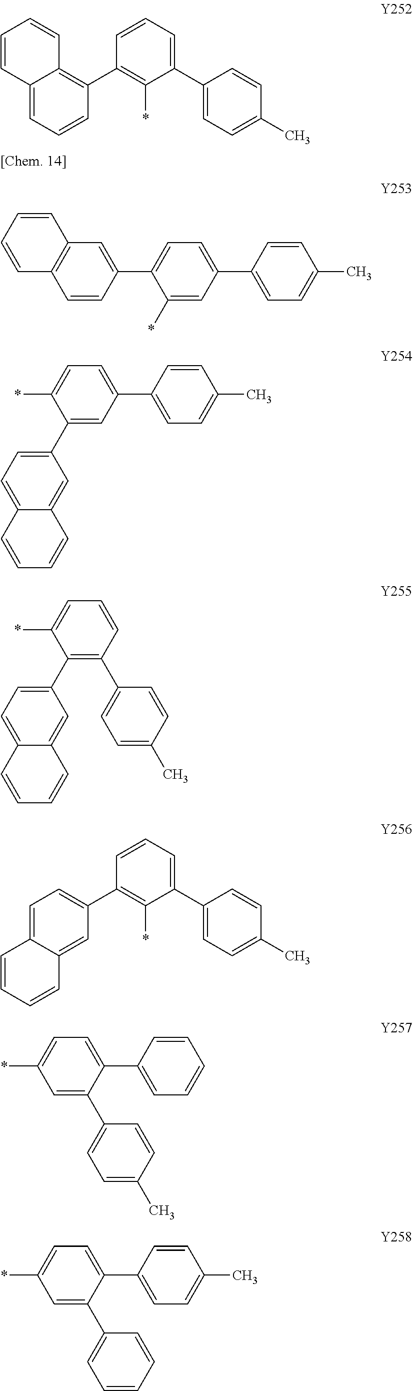

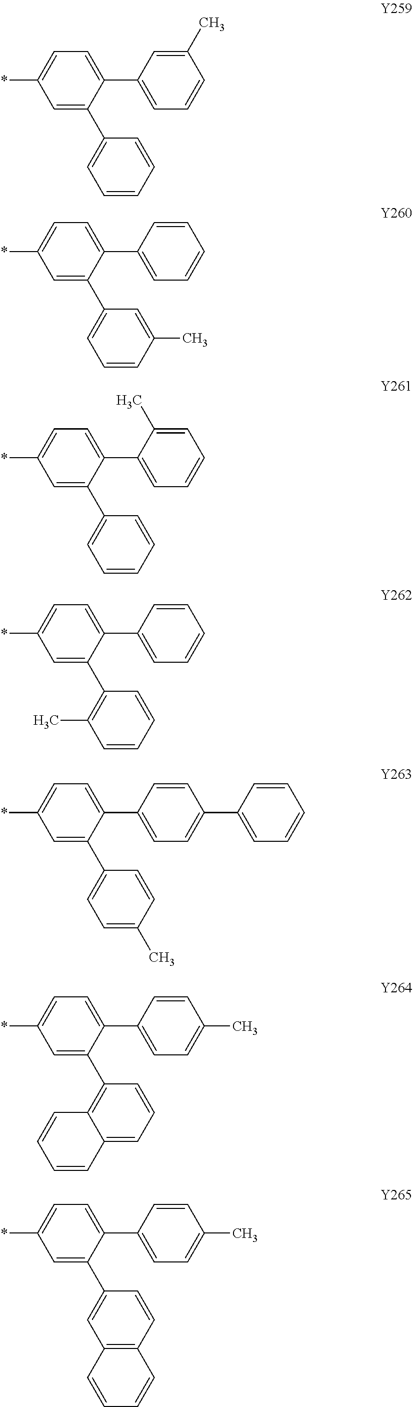

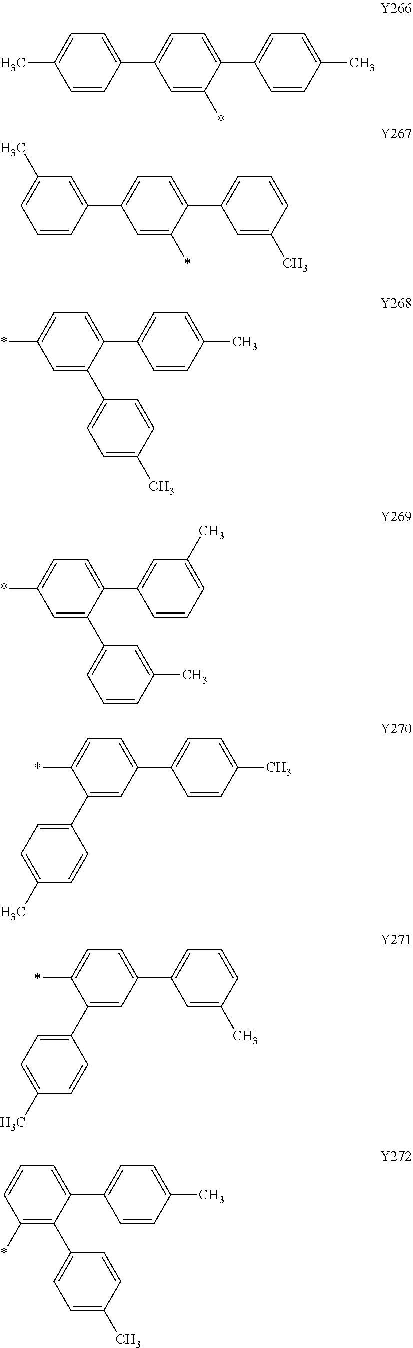

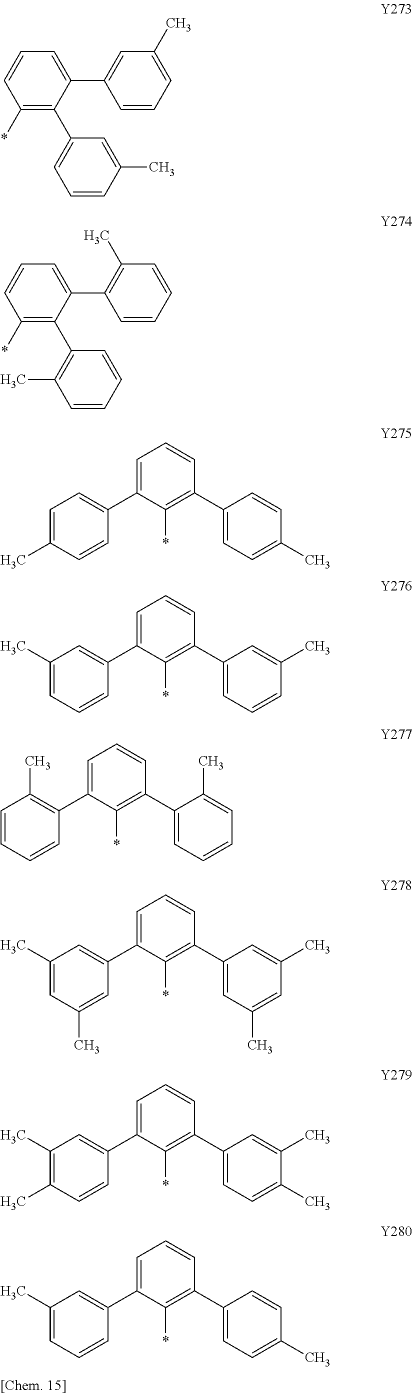

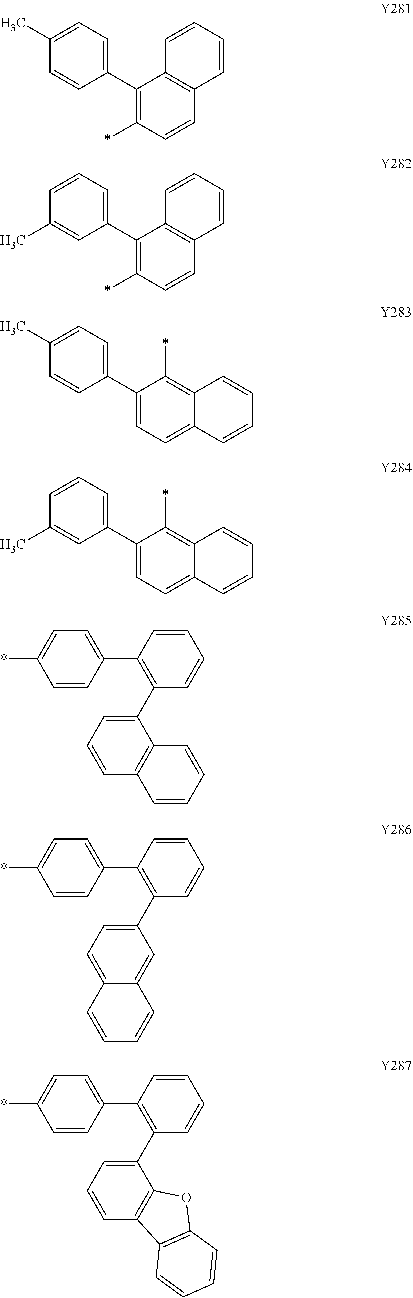

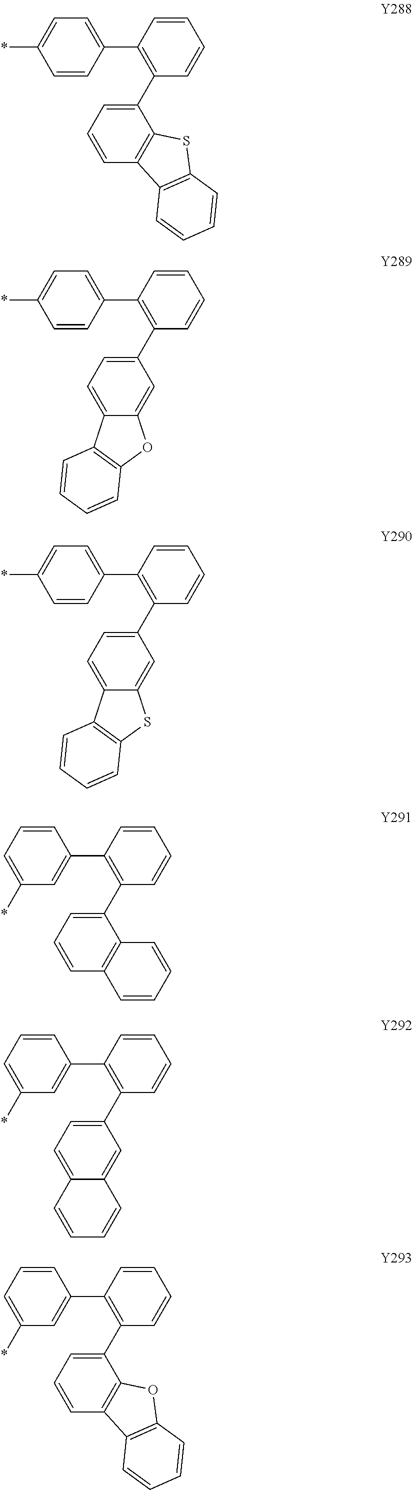

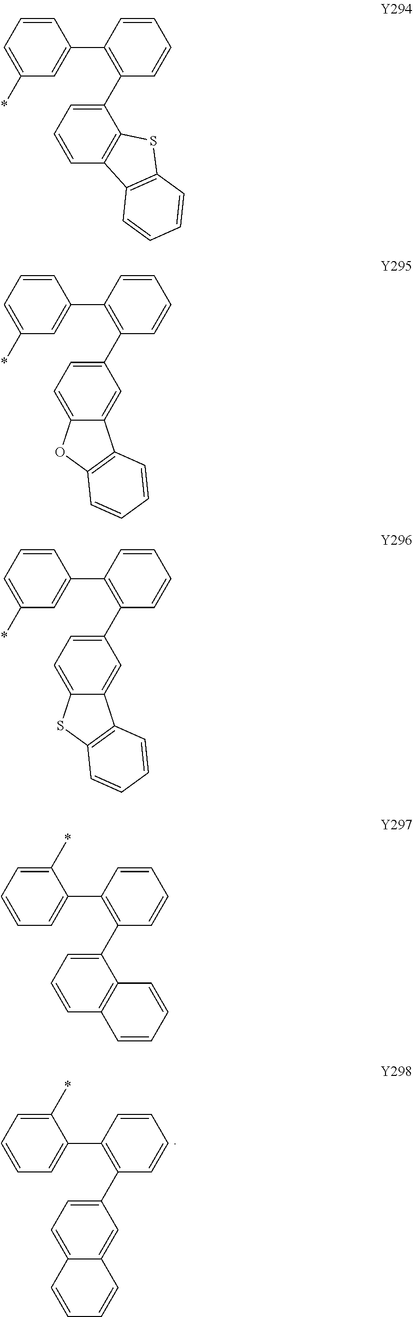

6. The transverse current suppressing material for the organic electroluminescent element according to claim 1, wherein both Ar¹ and Ar² each independently denote a group represented by any one of the formulae (Y2) to (Y9), (Y11) to (Y18) and (Y21) to (Y298).

7. A carbazole compound represented by the formula (22) or (23): 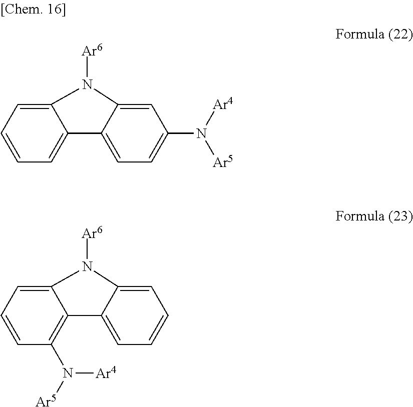
wherein
Ar⁶ each independently denotes a group selected from the formulae (24) to (45): 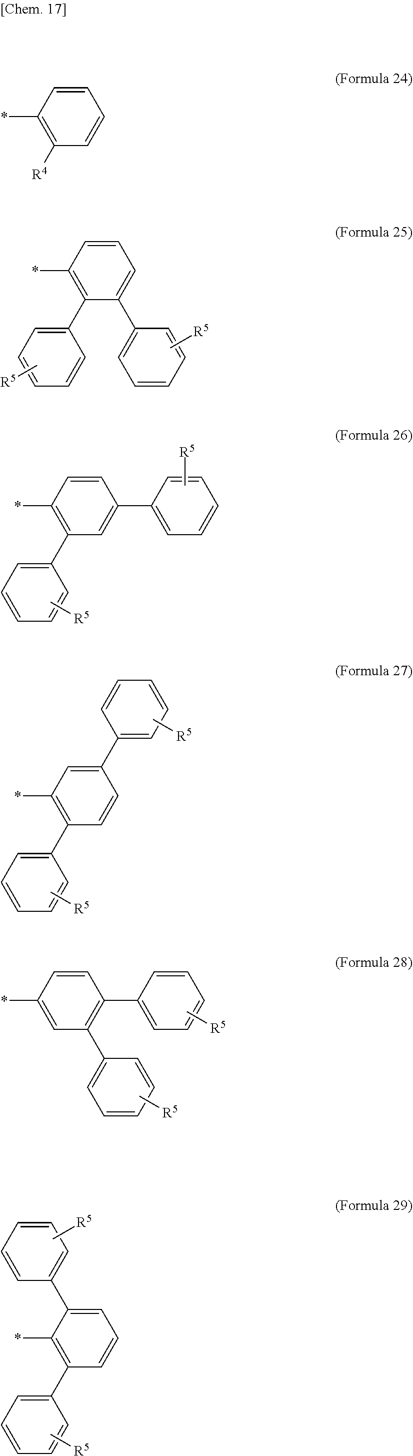 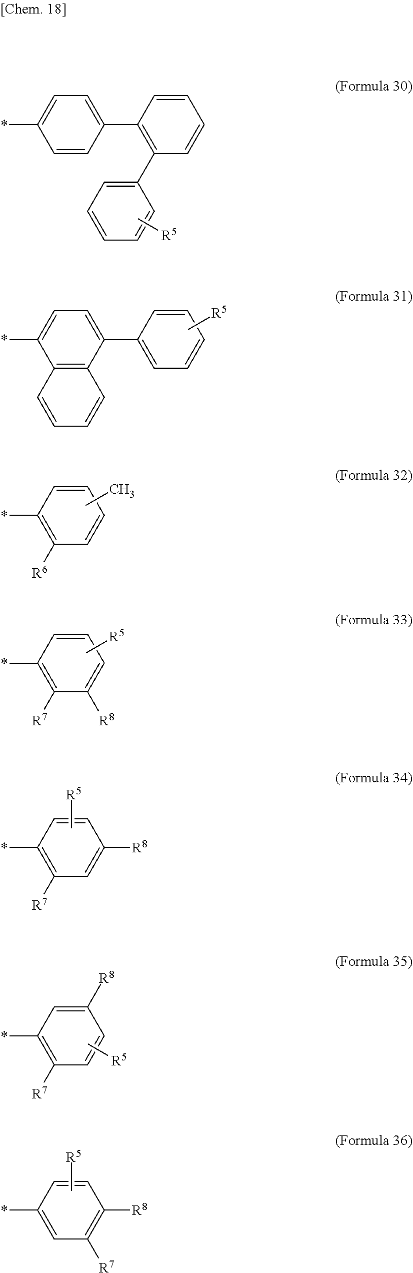 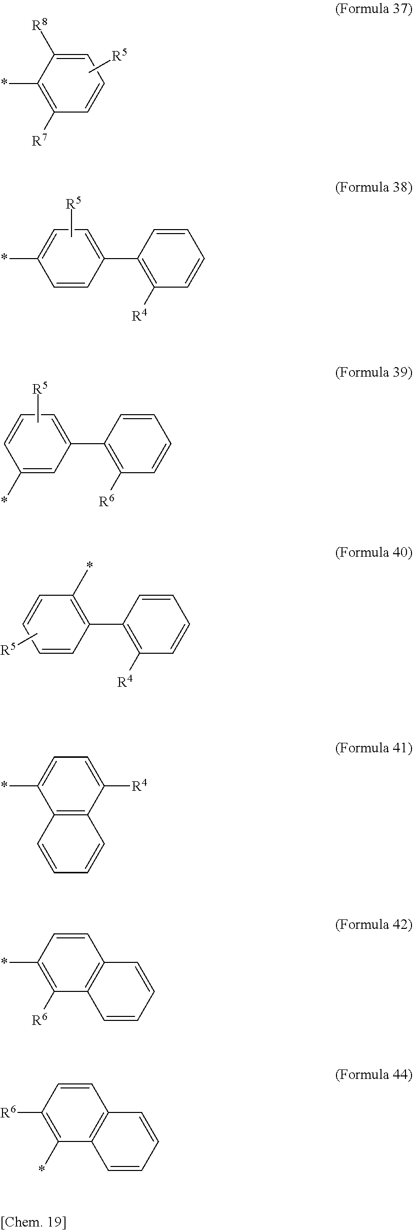 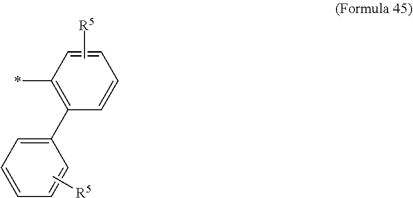
wherein
R⁴ denotes a biphenylyl group, a naphthyl group, a phenanthryl group, a dibenzofuranyl group or a dibenzothienyl group each optionally substituted with a methyl group,
R⁵ each independently denotes a methyl group or a hydrogen atom,
R⁶ denotes a phenyl group, a biphenylyl group, a naphthyl group, a phenanthryl group, a dibenzofuranyl group or a dibenzothienyl group each optionally substituted with a methyl group,
R⁷ and R⁸ each independently denote a phenyl group, a biphenylyl group, a naphthyl group, a phenanthryl group, a dibenzofuranyl group or a dibenzothienyl group each optionally substituted with a methyl group, and at least one of R⁷ and R⁸ denotes a biphenylyl group, a naphthyl group, a phenanthryl group, a dibenzofuranyl group or a dibenzothienyl group each optionally substituted with a methyl group,
in the formulae (22) and (23), when Ar⁶ denotes a group selected from the formulae (24) to (31),
Ar⁵ denotes a group selected from the formulae (24) to (45), and Ar⁴ denotes an optionally substituted monocyclic, linked or fused aromatic hydrocarbon group with 6 to 30 carbon atoms or a group represented by an optionally substituted monocyclic, linked or fused heteroaromatic group with 3 to 30 carbon atoms, and
in the formulae (22) and (23), when Ar⁶ denotes a group selected from the formulae (32) to (44),
Ar⁴ and Ar⁵ each independently denote a group selected from the formulae (24) to (45), an optionally substituted monocyclic, linked or fused aromatic hydrocarbon group with 6 to 30 carbon atoms or a group represented by an optionally substituted monocyclic, linked or fused heteroaromatic group with 3 to 30 carbon atoms.

8. The carbazole compound according to claim 7, wherein
Ar⁴ denotes
(iv) a phenyl group, a biphenylyl group, a terphenylyl group, a naphthyl group, a fluorenyl group, a spirobifluorenyl group, a benzofluorenyl group, a phenanthryl group, a fluoranthenyl group, a triphenylenyl group, an anthryl group, a pyrenyl group, a dibenzofuranyl group or a dibenzothienyl group,
(v) the group represented by (iv) substituted with one or more groups selected from the group consisting of a methyl group, an ethyl group, a methoxy group, an ethoxy group, a cyano group, a deuterium atom, a fluorine atom, a phenyl group, a biphenylyl group, a naphthyl group, a phenanthryl group, a triphenylsilyl group, a carbazolyl group, a dibenzothienyl group and a dibenzofuranyl group or
(vi) a group represented by any one of the formulae (24) to (41).

9. The carbazole compound according to claim 7, wherein Ar⁶ denotes a group represented by any one of (Z1) to (Z209).

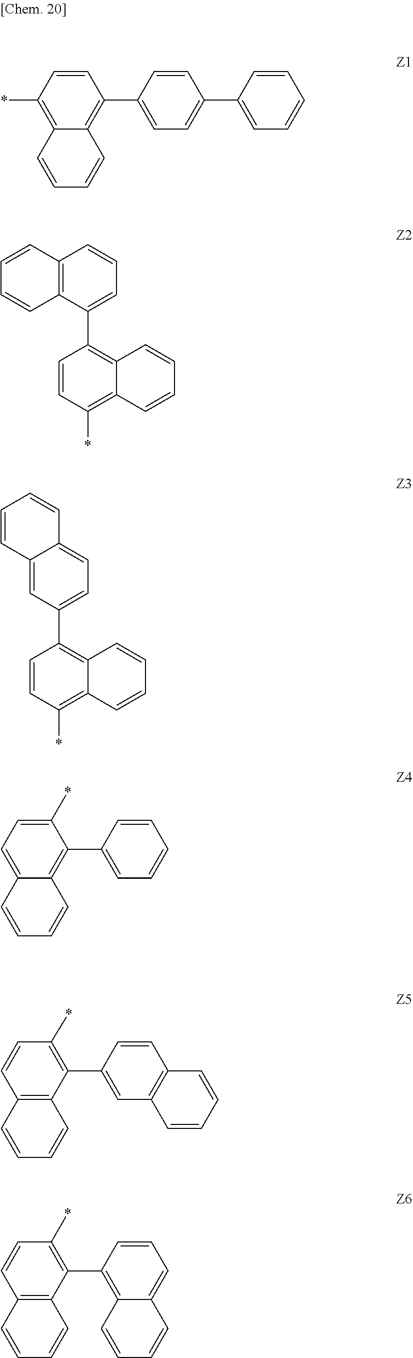 [SMILES: *c1ccc(-c2ccc(-c3ccccc3)cc2)c2ccccc12 ; *c1ccc(-c2cccc3ccccc23)c2ccccc12 ; *c1ccc(-c2ccc3ccccc3c2)c2ccccc12 ; *c1ccc2ccccc2c1-c1ccccc1 ; *c1ccc2ccccc2c1-c1ccc2ccccc2c1 ; *c1ccc2ccccc2c1-c1cccc2ccccc12]

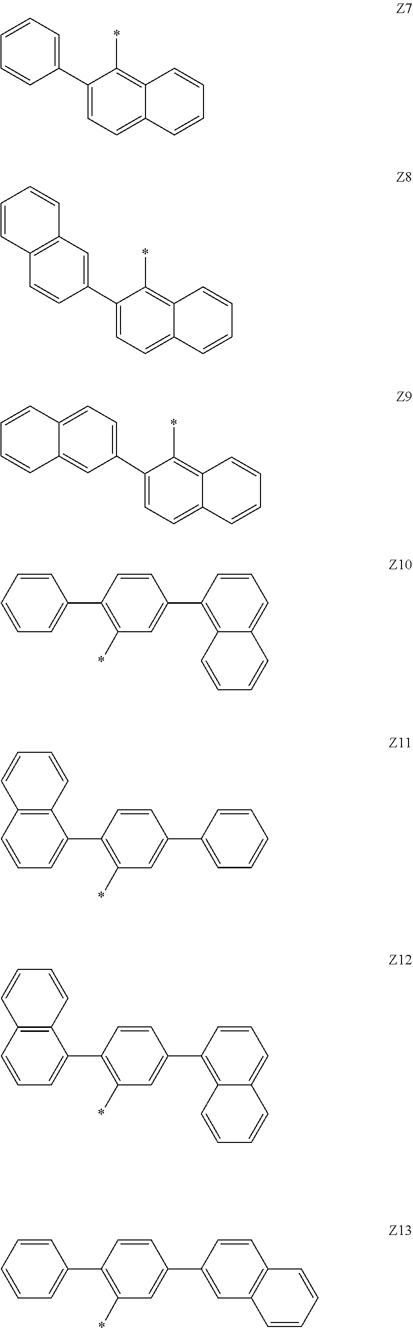

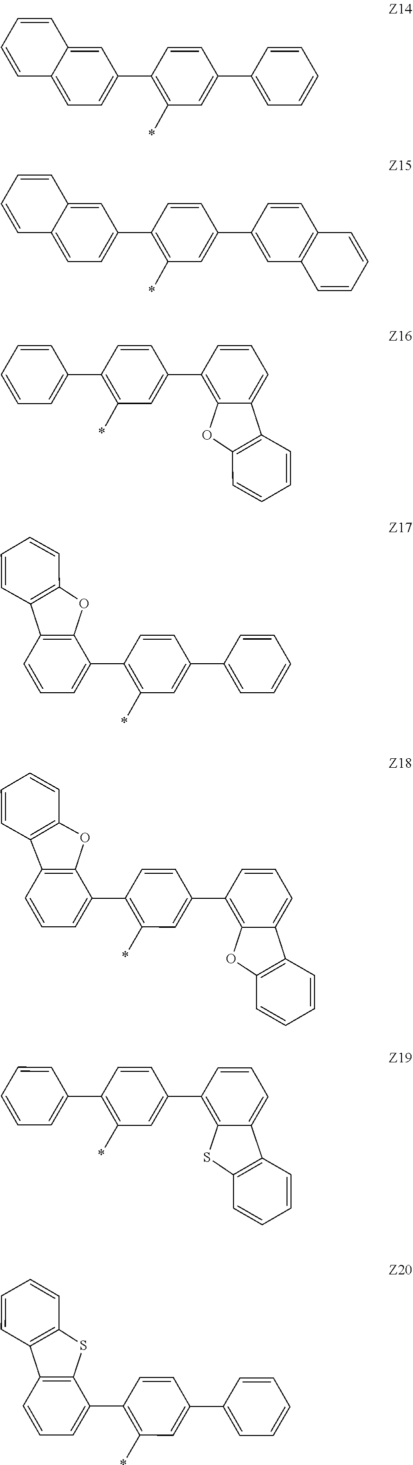

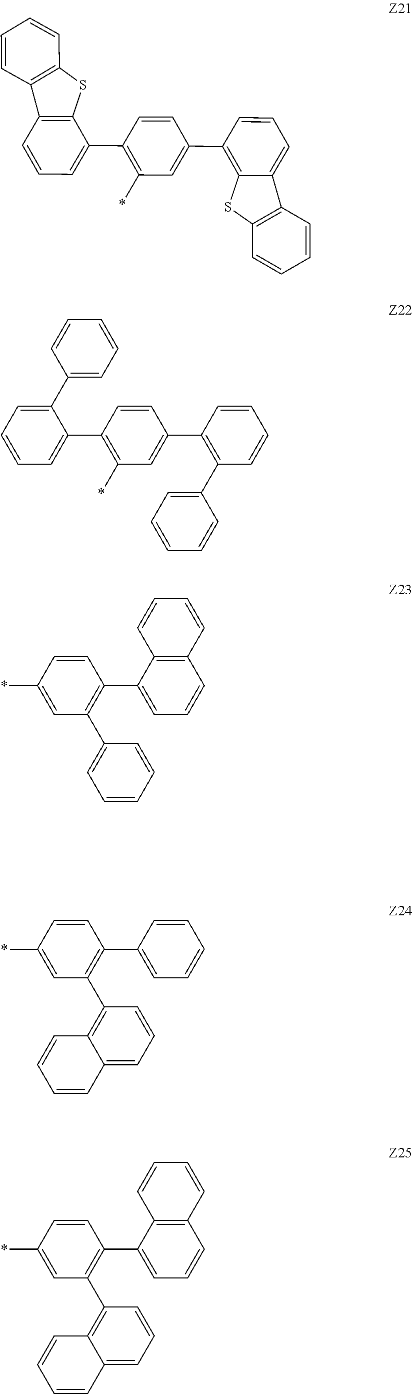 [SMILES: *c1cc(-c2cccc3c2sc2ccccc23)ccc1-c1cccc2c1sc1ccccc12 ; *c1cc(-c2ccccc2-c2ccccc2)ccc1-c1ccccc1-c1ccccc1 ; *c1ccc(-c2cccc3ccccc23)c(-c2ccccc2)c1 ; *c1ccc(-c2ccccc2)c(-c2cccc3ccccc23)c1 ; *c1ccc(-c2cccc3ccccc23)c(-c2cccc3ccccc23)c1]

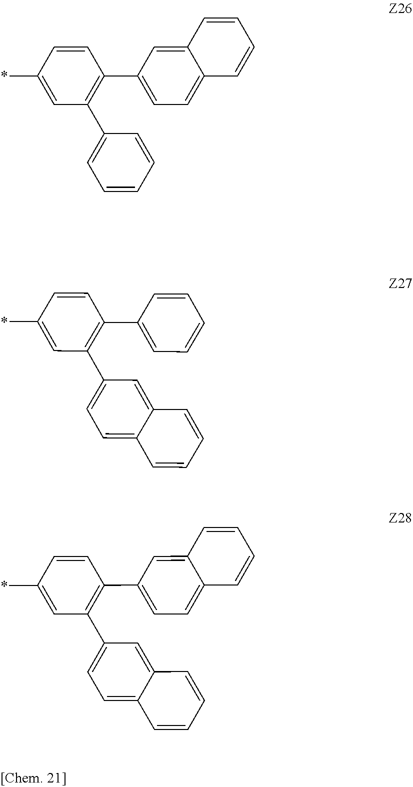 [SMILES: *c1ccc(-c2ccc3ccccc3c2)c(-c2ccccc2)c1 ; *c1ccc(-c2ccccc2)c(-c2ccc3ccccc3c2)c1 ; *c1ccc(-c2ccc3ccccc3c2)c(-c2ccc3ccccc3c2)c1]

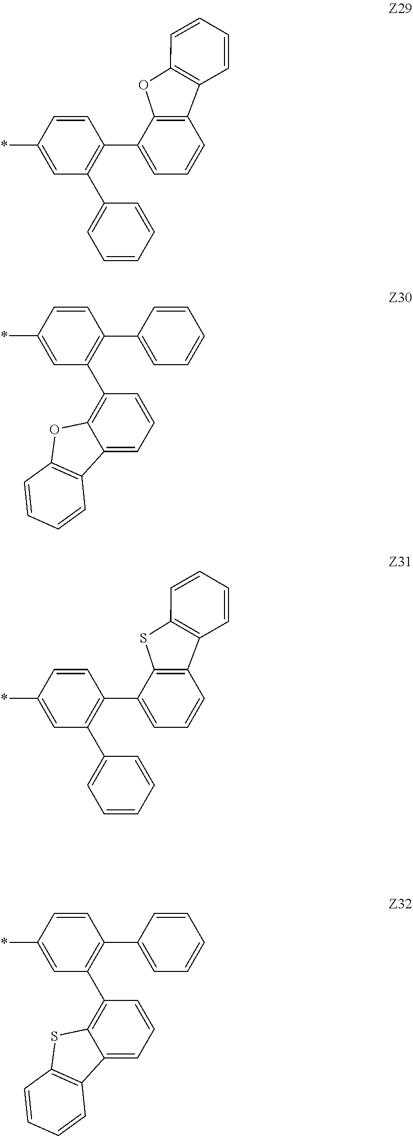 [SMILES: *c1ccc(-c2cccc3c2oc2ccccc23)c(-c2ccccc2)c1 ; *c1ccc(-c2ccccc2)c(-c2cccc3c2oc2ccccc23)c1 ; *c1ccc(-c2cccc3c2sc2ccccc23)c(-c2ccccc2)c1 ; *c1ccc(-c2ccccc2)c(-c2cccc3c2sc2ccccc23)c1]

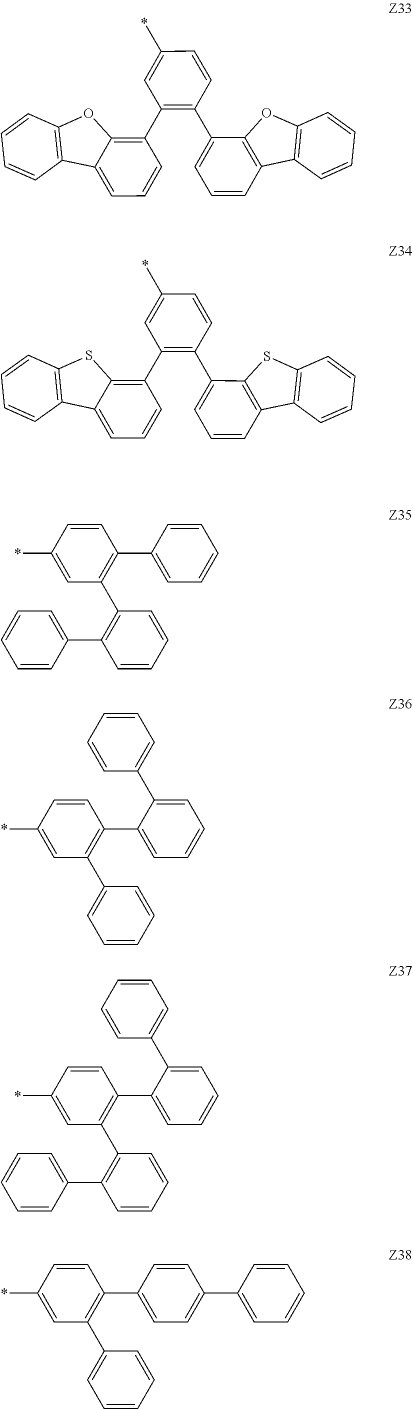

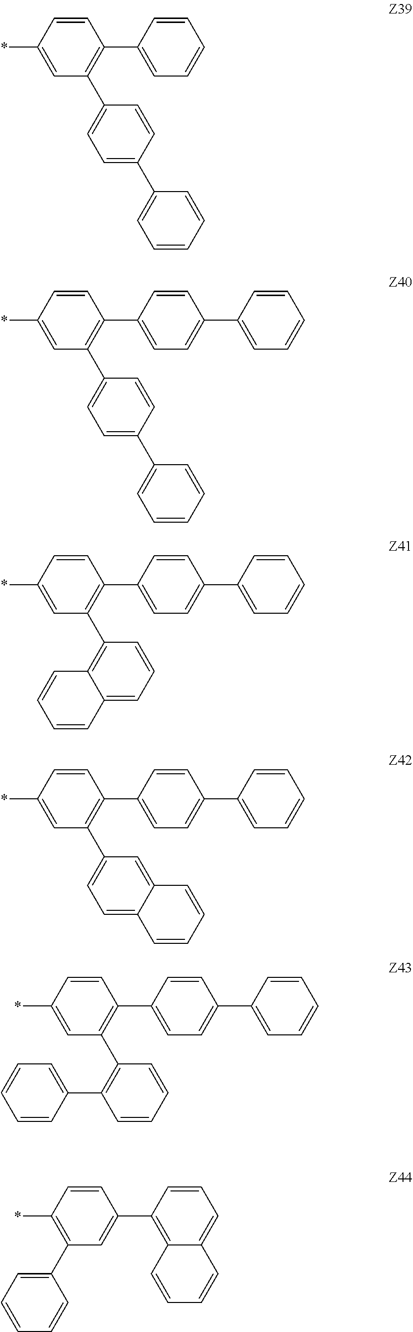

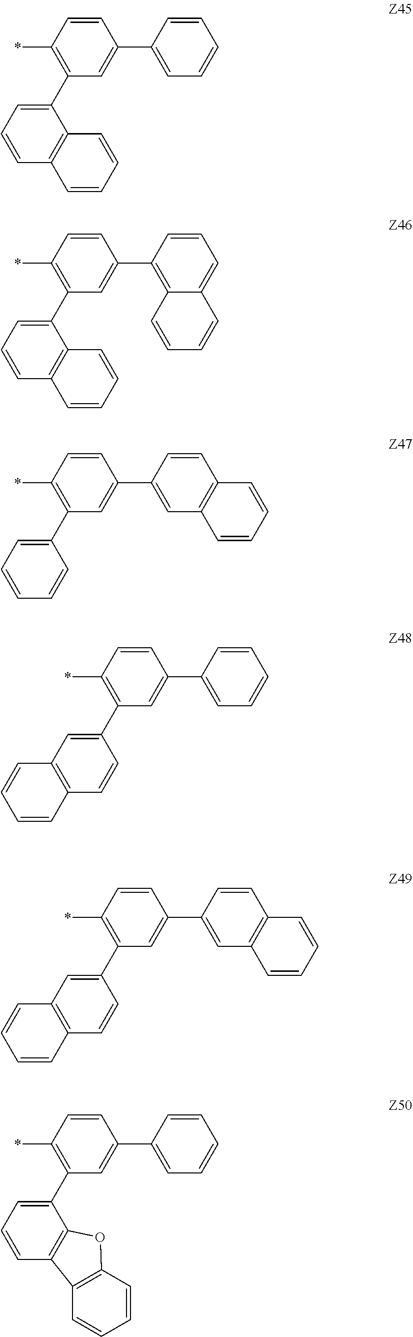

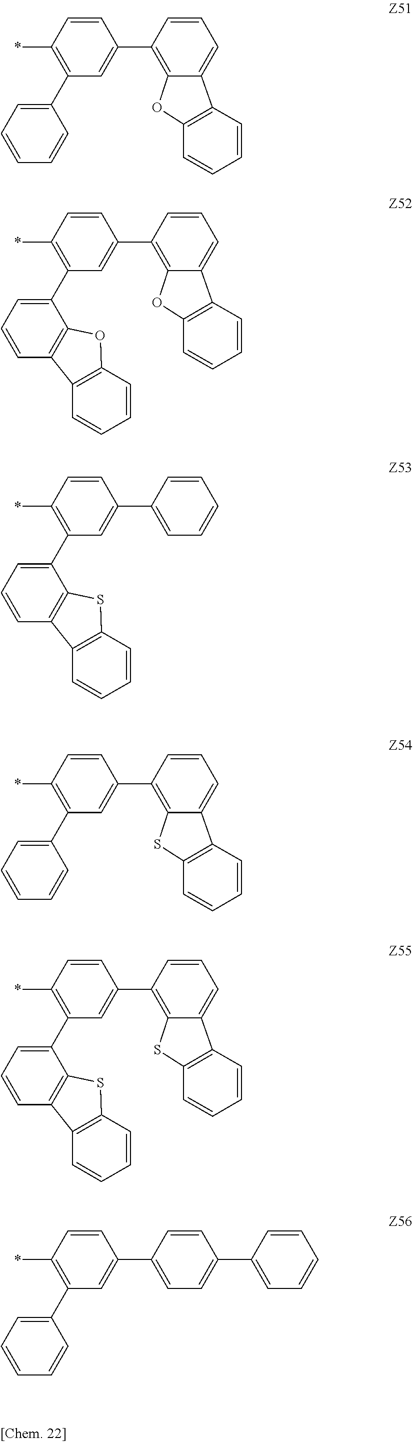

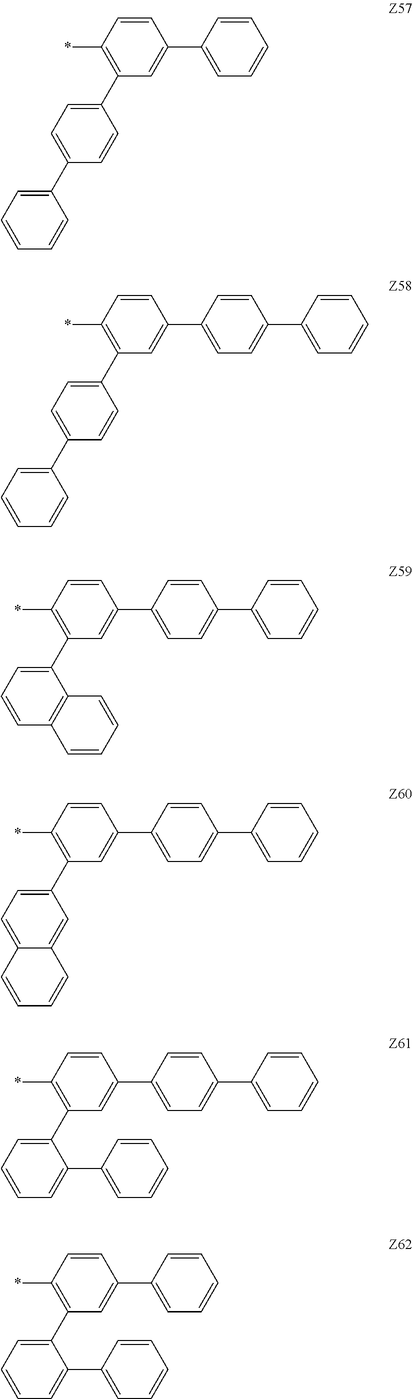 [SMILES: *c1ccc(-c2ccccc2)cc1-c1ccc(-c2ccccc2)cc1 ; *c1ccc(-c2ccc(-c3ccccc3)cc2)cc1-c1ccc(-c2ccccc2)cc1 ; *c1ccc(-c2ccc(-c3ccccc3)cc2)cc1-c1cccc2ccccc12 ; *c1ccc(-c2ccc(-c3ccccc3)cc2)cc1-c1ccc2ccccc2c1 ; *c1ccc(-c2ccc(-c3ccccc3)cc2)cc1-c1ccccc1-c1ccccc1 ; *c1ccc(-c2ccccc2)cc1-c1ccccc1-c1ccccc1]

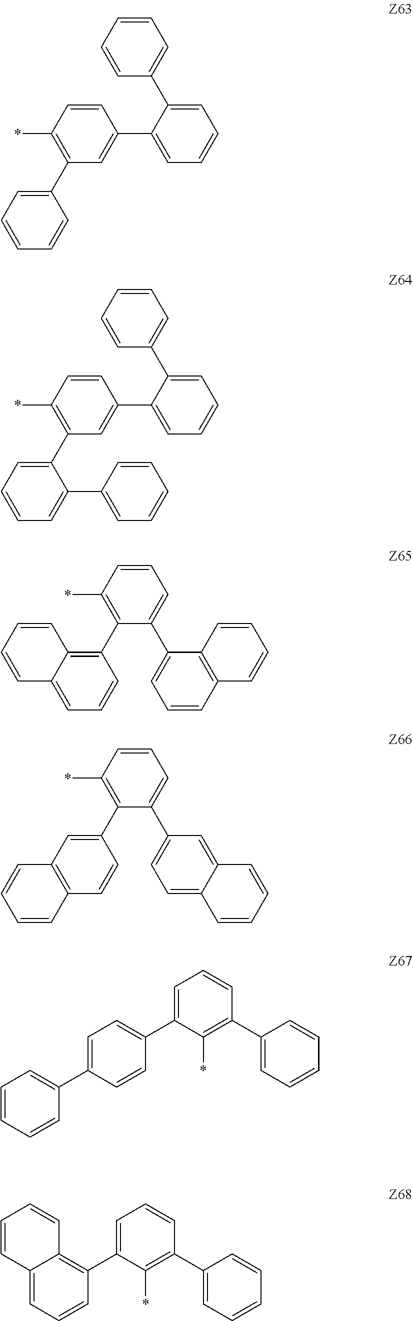

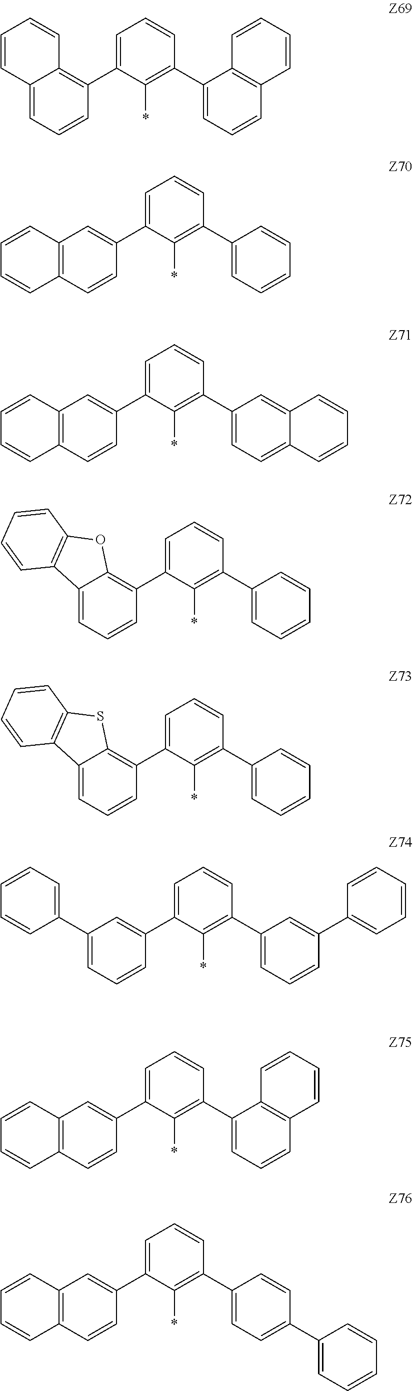

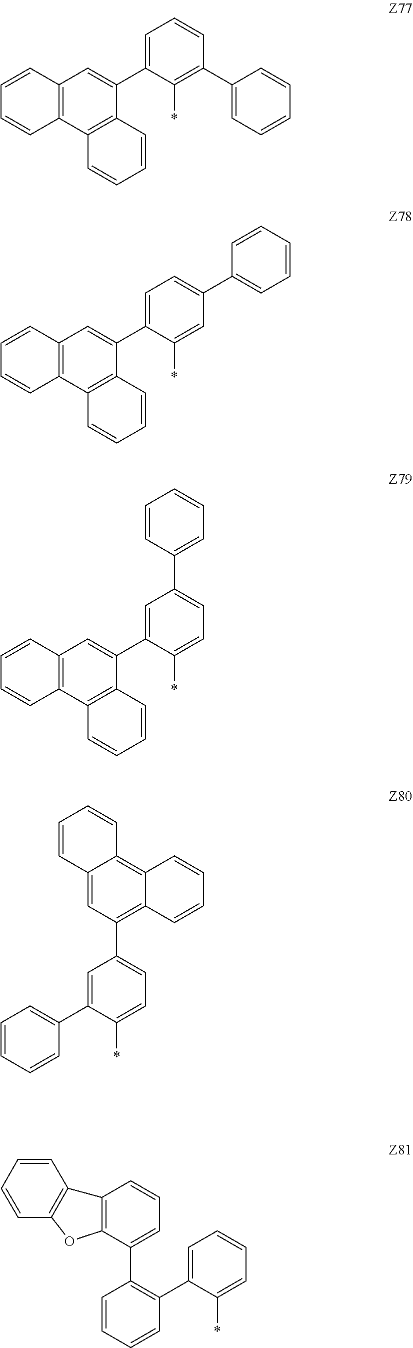 [SMILES: *c1c(-c2ccccc2)cccc1-c1cc2ccccc2c2ccccc12 ; *c1cc(-c2ccccc2)ccc1-c1cc2ccccc2c2ccccc12 ; *c1ccc(-c2ccccc2)cc1-c1cc2ccccc2c2ccccc12 ; *c1ccc(-c2cc3ccccc3c3ccccc23)cc1-c1ccccc1 ; *c1ccccc1-c1ccccc1-c1cccc2c1oc1ccccc12]

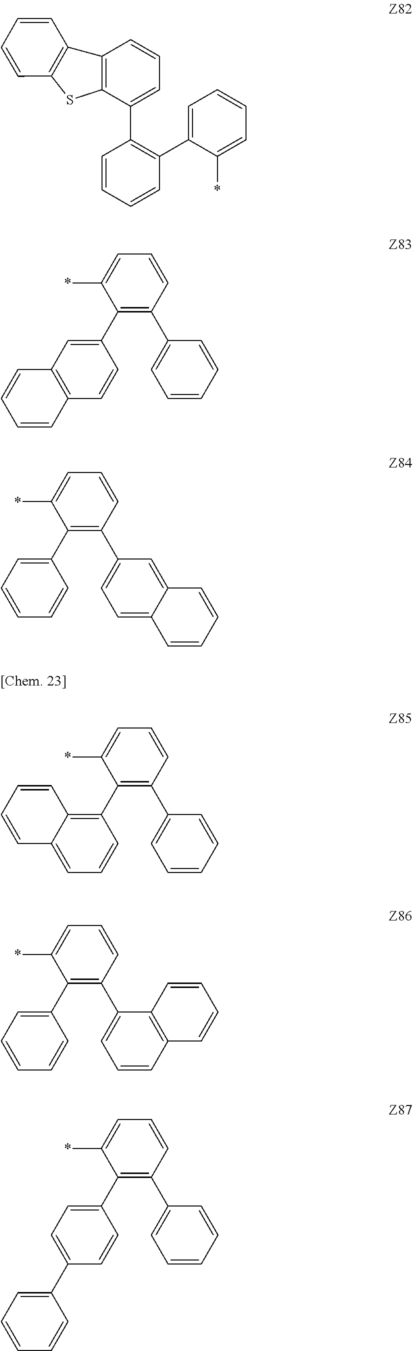 [SMILES: *c1ccccc1-c1ccccc1-c1cccc2c1sc1ccccc12 ; *c1cccc(-c2ccccc2)c1-c1ccc2ccccc2c1 ; *c1cccc(-c2ccc3ccccc3c2)c1-c1ccccc1 ; *c1cccc(-c2ccccc2)c1-c1cccc2ccccc12 ; *c1cccc(-c2cccc3ccccc23)c1-c1ccccc1 ; *c1cccc(-c2ccccc2)c1-c1ccc(-c2ccccc2)cc1]

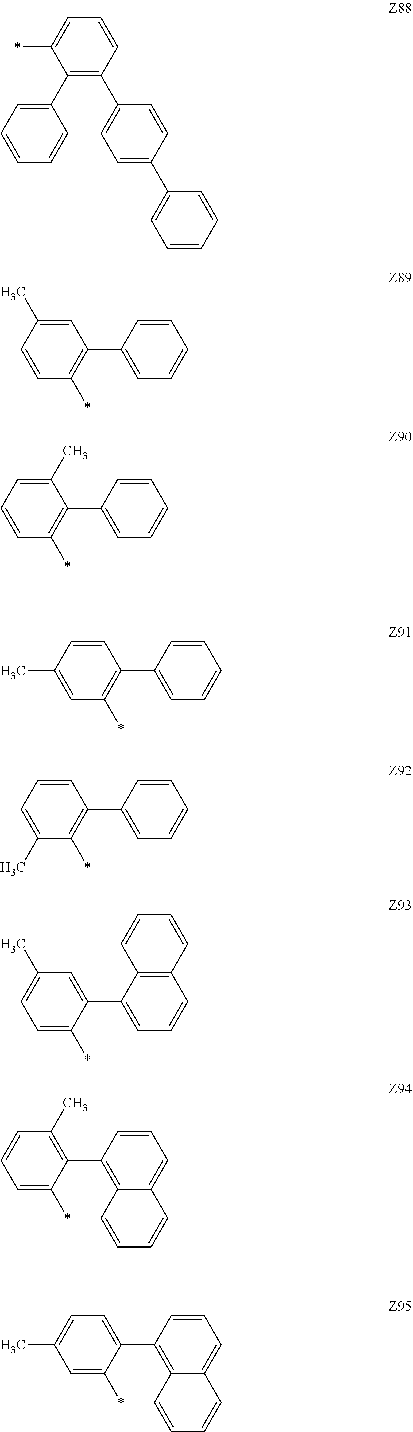

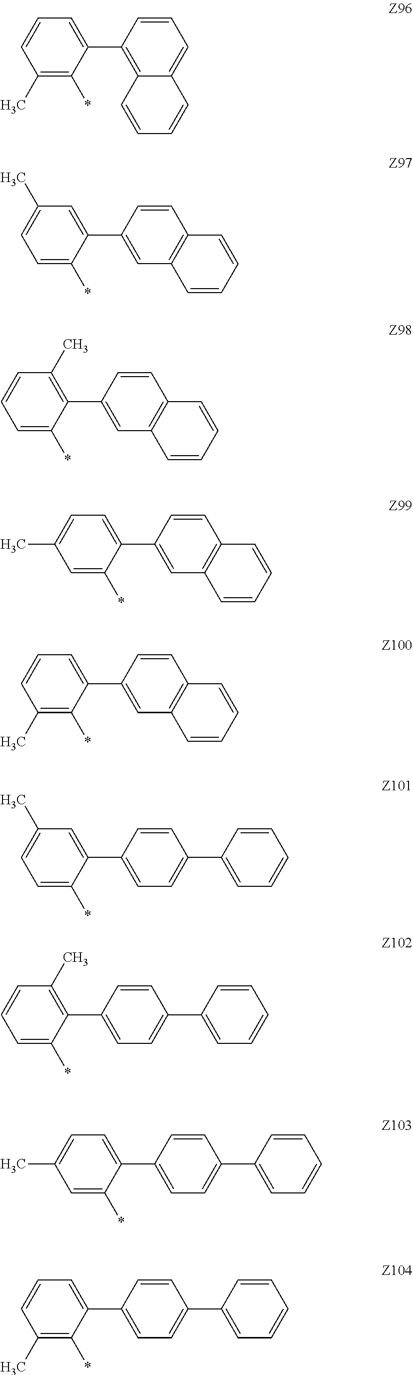

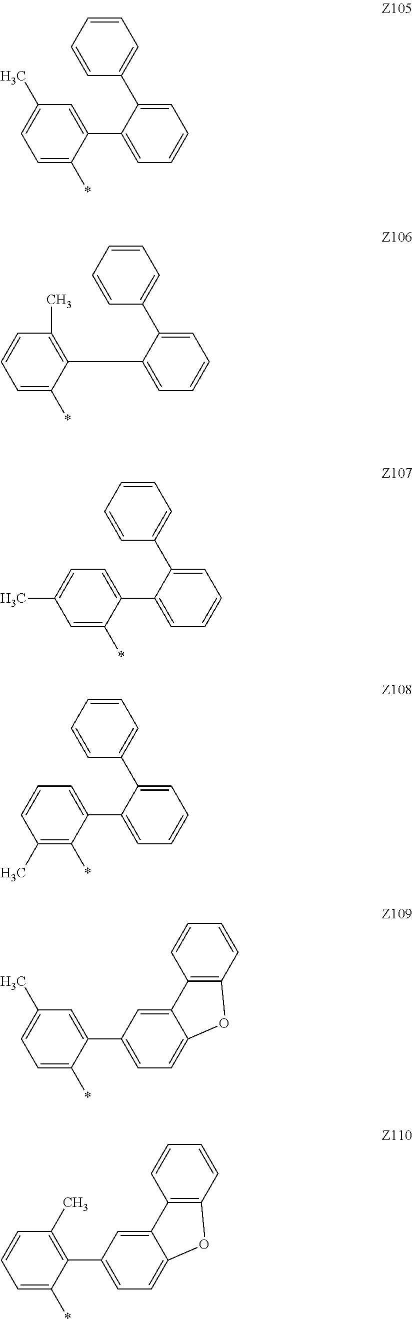

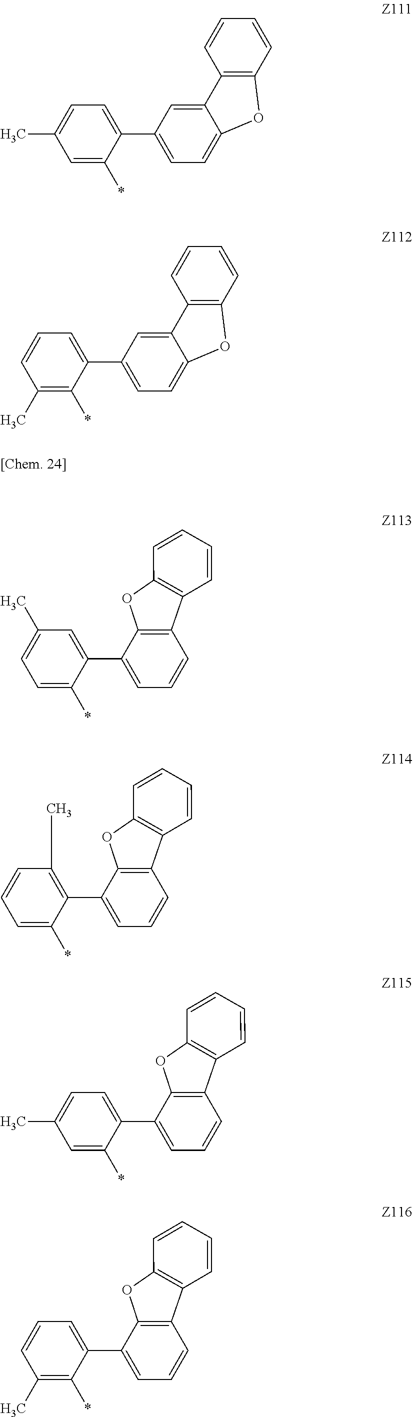 [SMILES: *c1cc(C)ccc1-c1ccc2oc3ccccc3c2c1 ; *c1c(C)cccc1-c1ccc2oc3ccccc3c2c1 ; *c1ccc(C)cc1-c1cccc2c1oc1ccccc12 ; *c1cccc(C)c1-c1cccc2c1oc1ccccc12 ; *c1cc(C)ccc1-c1cccc2c1oc1ccccc12 ; *c1c(C)cccc1-c1cccc2c1oc1ccccc12]

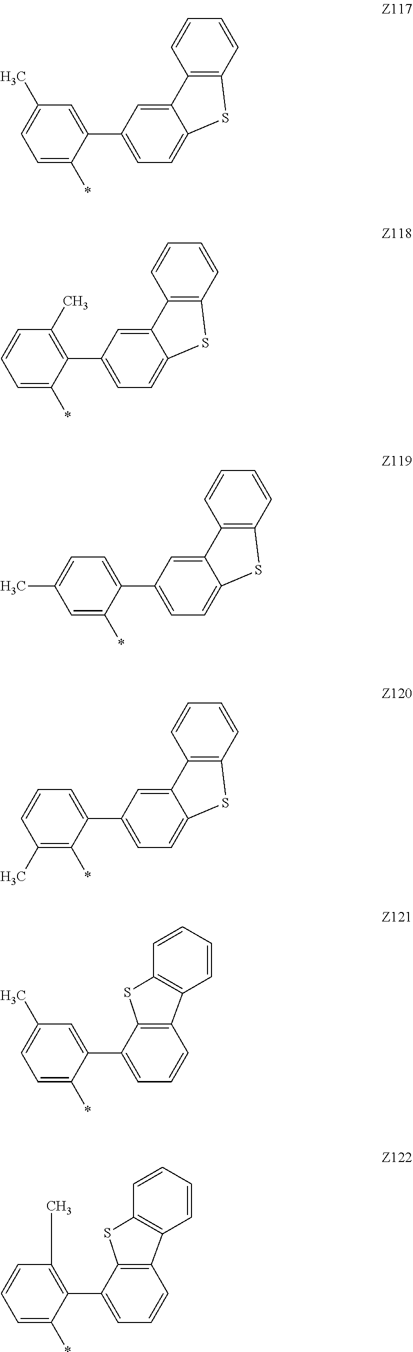 [SMILES: *c1ccc(C)cc1-c1ccc2sc3ccccc3c2c1 ; *c1cccc(C)c1-c1ccc2sc3ccccc3c2c1 ; *c1cc(C)ccc1-c1ccc2sc3ccccc3c2c1 ; *c1c(C)cccc1-c1ccc2sc3ccccc3c2c1 ; *c1ccc(C)cc1-c1cccc2c1sc1ccccc12 ; *c1cccc(C)c1-c1cccc2c1sc1ccccc12]

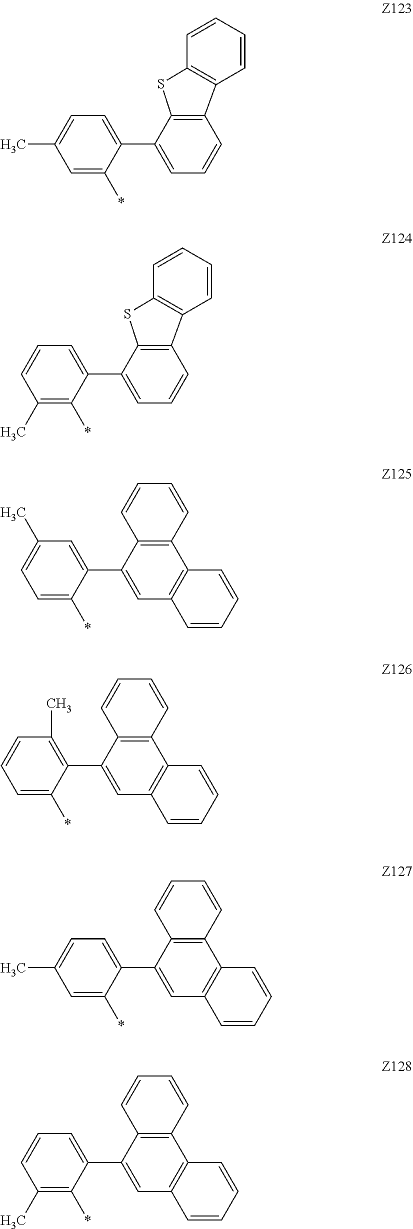 [SMILES: *c1cc(C)ccc1-c1cccc2c1sc1ccccc12 ; *c1c(C)cccc1-c1cccc2c1sc1ccccc12 ; *c1ccc(C)cc1-c1cc2ccccc2c2ccccc12 ; *c1cccc(C)c1-c1cc2ccccc2c2ccccc12 ; *c1cc(C)ccc1-c1cc2ccccc2c2ccccc12 ; *c1c(C)cccc1-c1cc2ccccc2c2ccccc12]

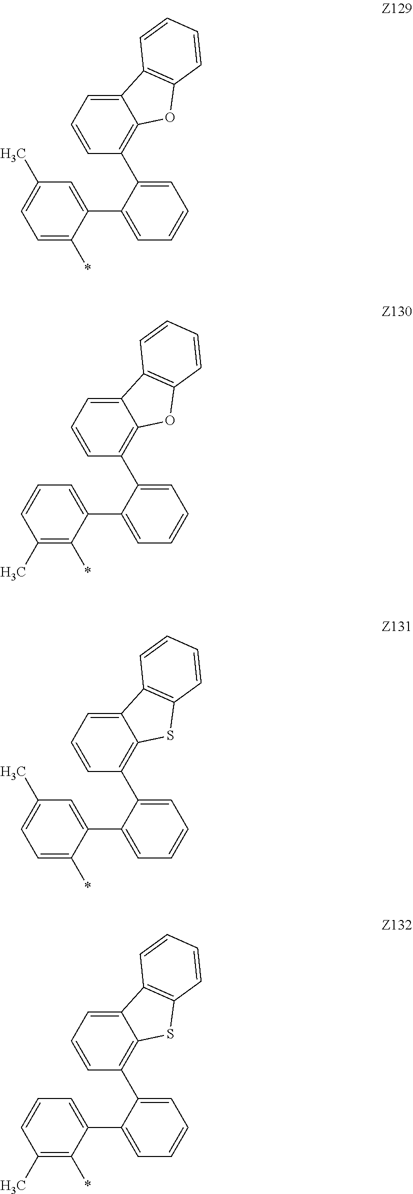 [SMILES: *c1ccc(C)cc1-c1ccccc1-c1cccc2c1oc1ccccc12 ; *c1c(C)cccc1-c1ccccc1-c1cccc2c1oc1ccccc12 ; *c1ccc(C)cc1-c1ccccc1-c1cccc2c1sc1ccccc12 ; *c1c(C)cccc1-c1ccccc1-c1cccc2c1sc1ccccc12]

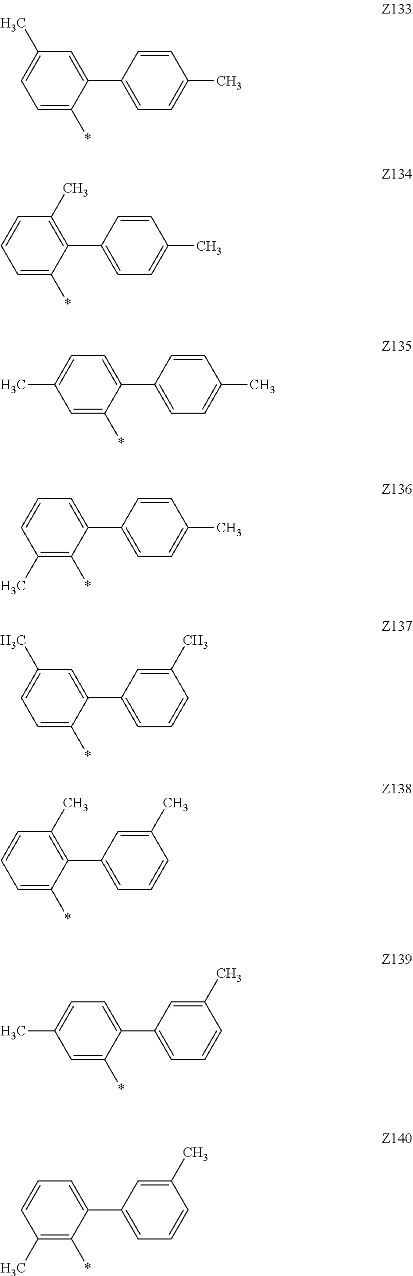

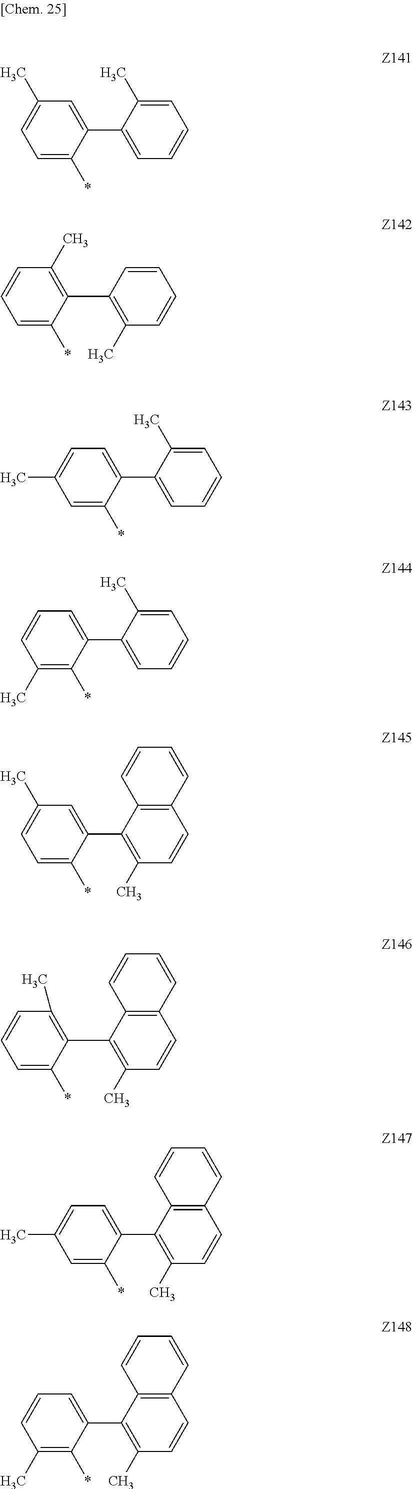

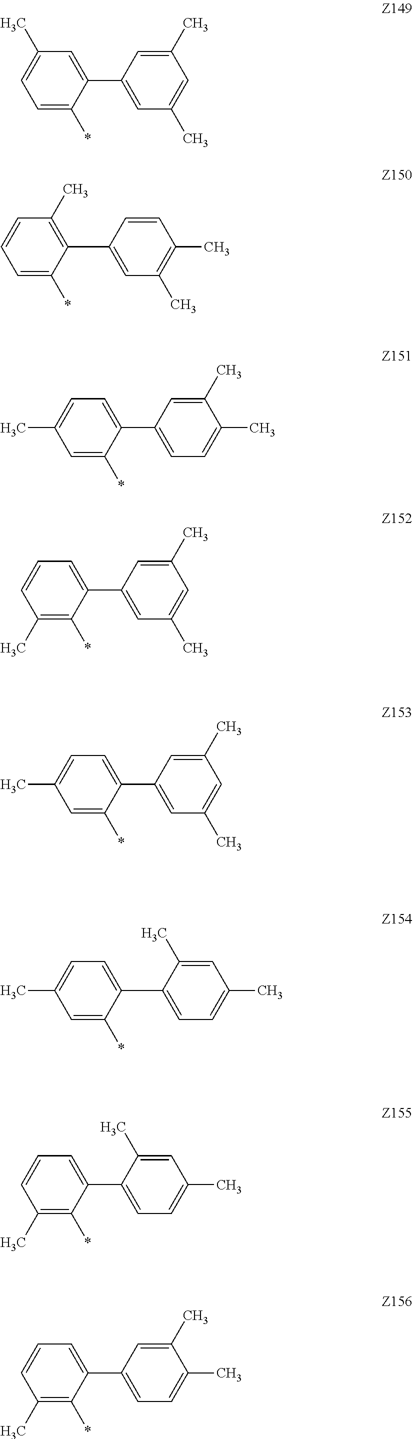

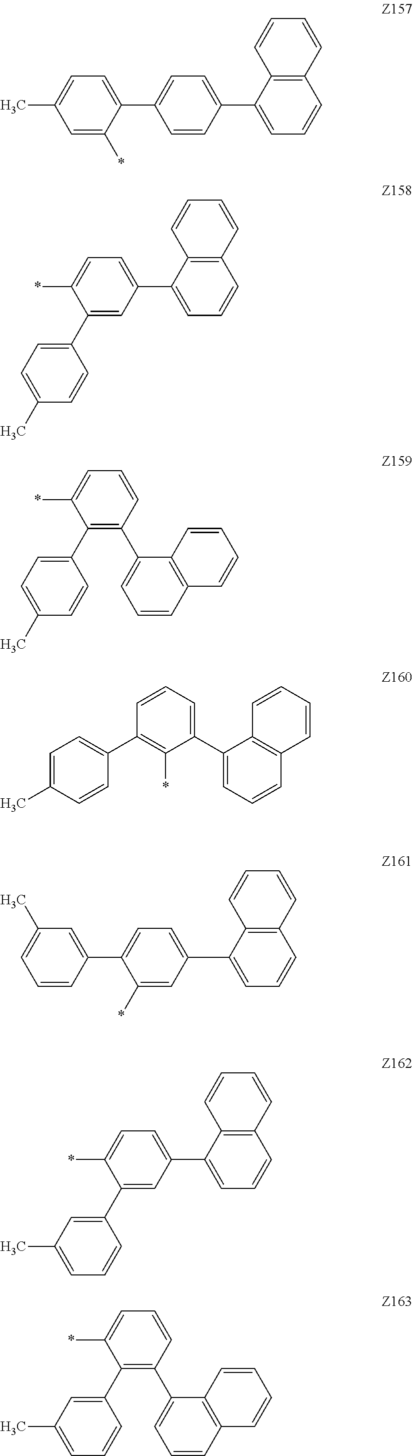

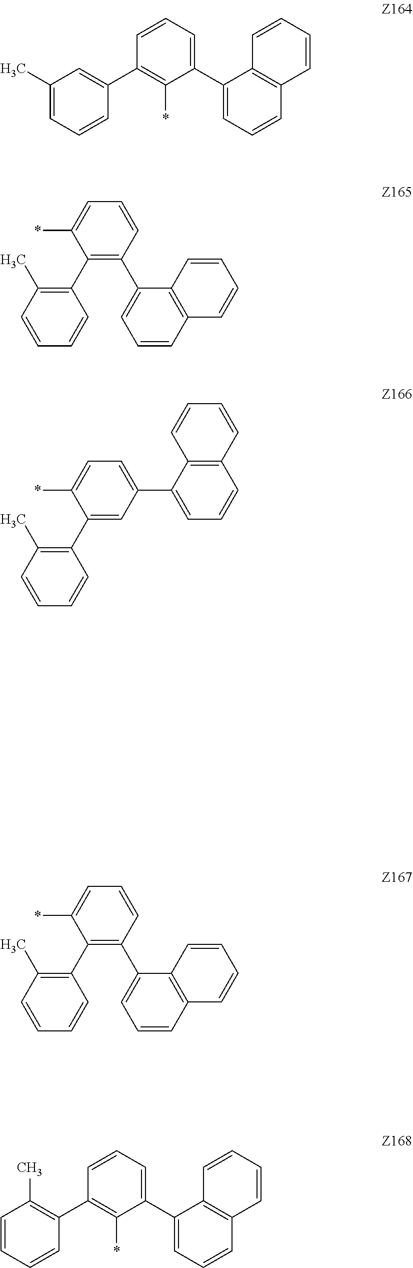 [SMILES: *c1c(-c2cccc(C)c2)cccc1-c1cccc2ccccc12 ; *c1cccc(-c2cccc3ccccc23)c1-c1ccccc1C ; *c1ccc(-c2cccc3ccccc23)cc1-c1ccccc1C ; *c1cccc(-c2cccc3ccccc23)c1-c1ccccc1C ; *c1c(-c2ccccc2C)cccc1-c1cccc2ccccc12]

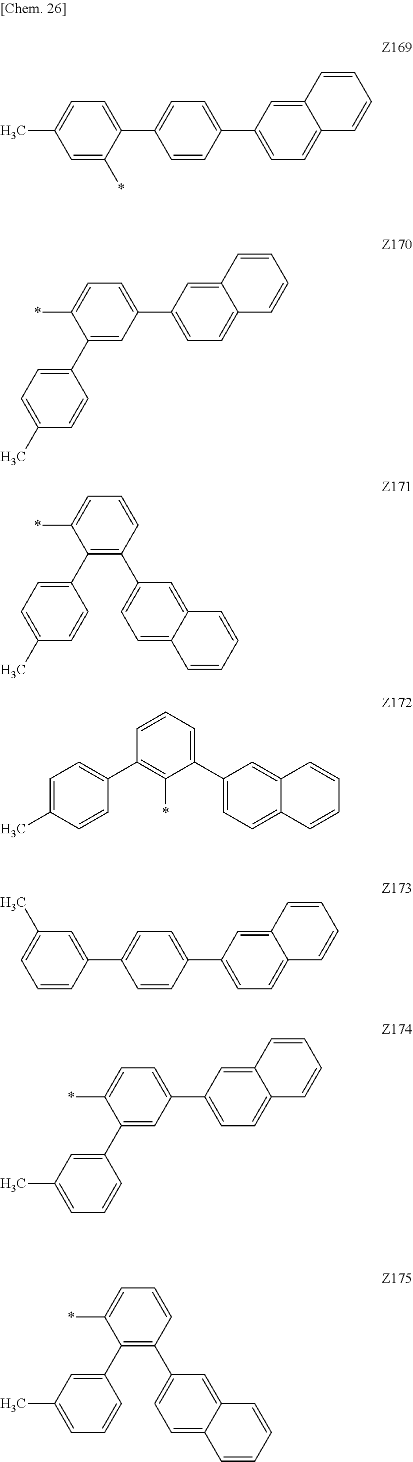

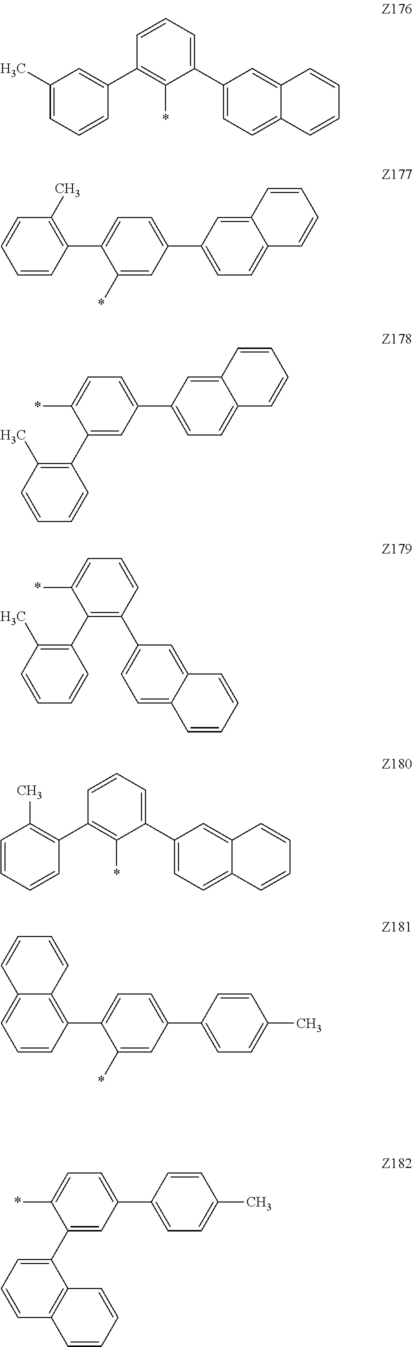

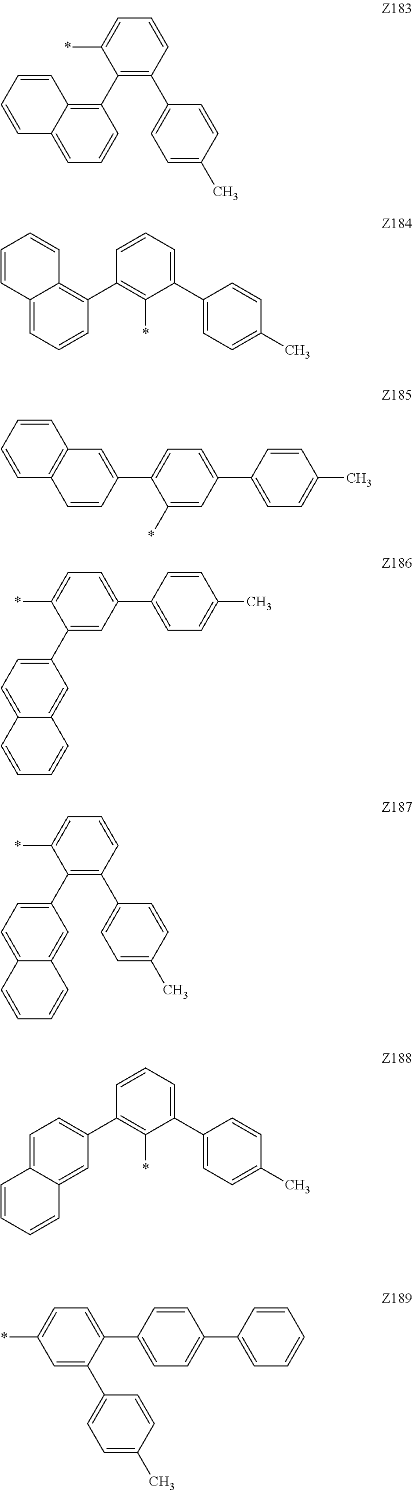

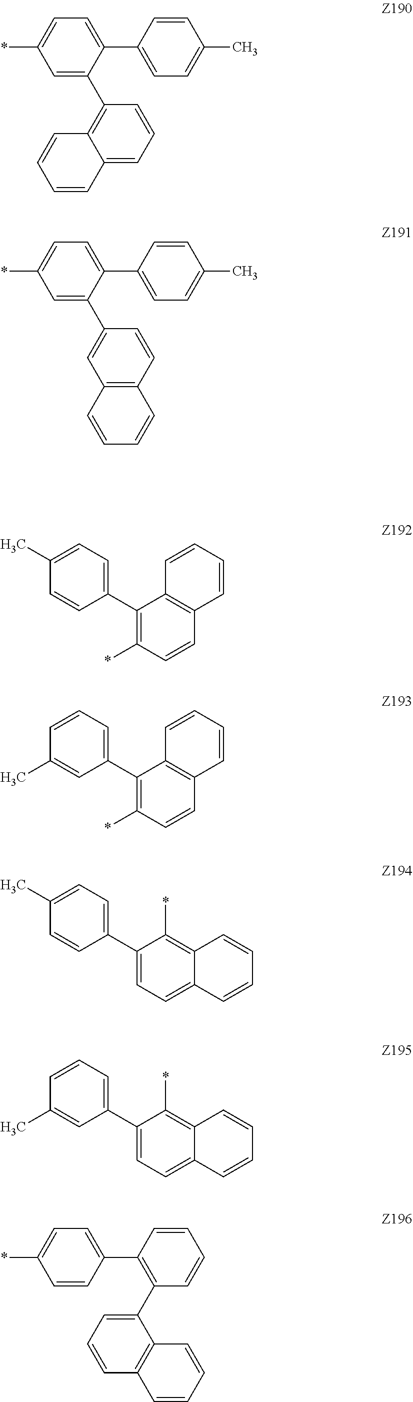

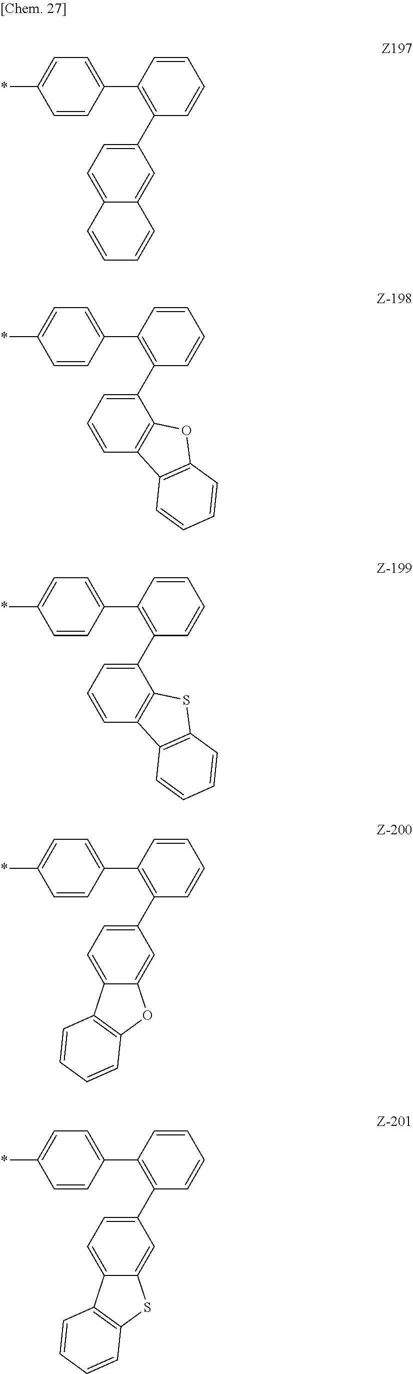

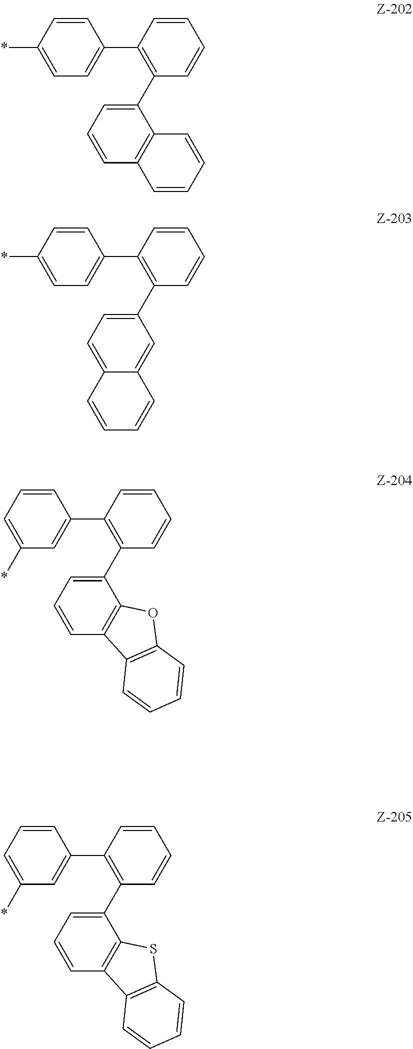

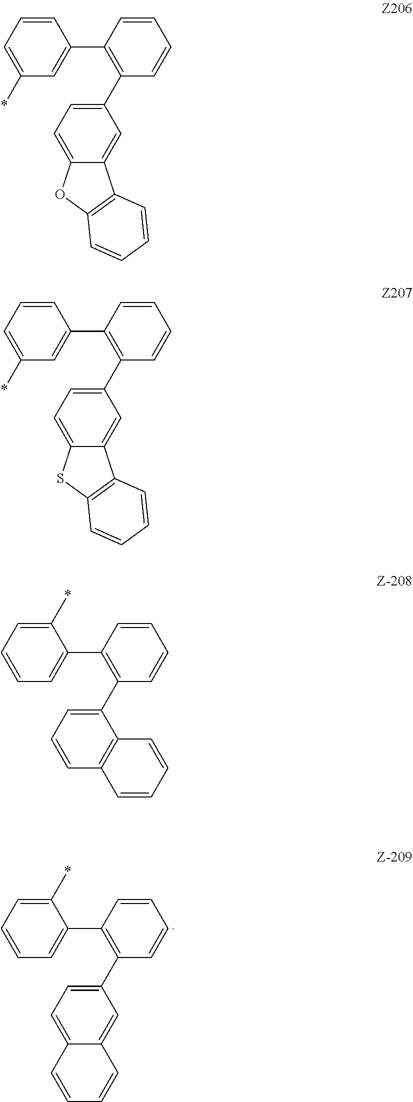

10. A hole-injection layer comprising:
a first compound; and
a second compound,
wherein the first compound is
the transverse current suppressing material according to claim 1, and
the second compound is an electron-accepting p-dopant.

11. The hole-injection layer according to claim 10, further comprising
a third compound,
wherein the third compound is a hole-transporting triarylamine compound.

12. The hole-injection layer according to claim 10, wherein the transverse current suppressing material constitutes 20% by mass or more and 99.5% by mass or less.

13. An organic electroluminescent element comprising a hole-injection layer,
wherein the hole-injection layer contains
the transverse current suppressing material according to claim 1.

14. The organic electroluminescent element according to claim 13, wherein the hole-injection layer comprises:
a first compound; and
a second compound,
wherein the first compound is the transverse current suppressing material, and
the second compound is an electron-accepting p-dopant.

15. The organic electroluminescent element according to claim 13, further comprising:
a hole-transport layer,
wherein the hole-transport layer contains
the transverse current suppressing material.

16. An organic electroluminescent element comprising:
a positive electrode;
a plurality of organic layers on the positive electrode; and
a negative electrode on the plurality of organic layers,
wherein one or more layers of the plurality of organic layers contain the carbazole compound according to claim 7.

17. A hole-injection layer comprising:
a first compound; and
a second compound,
wherein the first compound is
the carbazole compound according to claim 7, and
the second compound is an electron-accepting p-dopant.

18. The hole-injection layer according to claim 17, wherein the carbazole compound constitutes 20% by mass or more and 99.5% by mass or less.

19. An organic electroluminescent element comprising a hole-injection layer,
wherein the hole-injection layer contains the carbazole compound according to claim 7.

20. The organic electroluminescent element according to claim 19, further comprising:
a hole-transport layer,
wherein the hole-transport layer contains the carbazole compound.
